(19) United States
(12) Patent Application Publication
(10) Pub. No.: US 2018/0251839 A1
(43) Pub. Date: Sep. 6, 2018

(54) Compositions and Methods for the Diagnosis of Retinal Neovascularization

(71) Applicant: The Schepens Eye Research Institute, Inc., Boston, MA (US)

(72) Inventor: Neena B. Haider, Brookline, MA (US)

(21) Appl. No.: 15/782,790

(22) Filed: Oct. 12, 2017

Related U.S. Application Data
(63) Continuation of application No. 14/434,989, filed on Apr. 10, 2015, now abandoned.
(60) Provisional application No. 61/713,402, filed on Oct. 12, 2012.

(51) Int. Cl. C12Q 1/6883; C07K 14/435; C07K 14/705; C12N 9/12; C12N 9/48; C07K 14/79

(52) U.S. Cl. CPC C12Q 1/6883; C12Y 207/10001; C12Q 2600/158; C07K 14/435; C07K 14/705; C07K 14/79; C12N 9/12; C12N 9/48; C12Q 2600/156

(57) ABSTRACT

This application discloses compositions and methods for the diagnosis of retinal neovascularization.

### RELATED APPLICATIONS

This application is a continuation application of U.S. Ser. No. 14/434,989, filed on Apr. 10, 2015, which is a national stage application, filed under 35 U.S.C. § 371, of International Application No. PCT/US2013/064881, filed on Oct. 14, 2013, which claims benefit to and priority of U.S. Provisional Application No. 61/713,402 filed Oct. 12, 2012; the contents of which are hereby incorporated by reference in its entirety.

### INCORPORATION-BY-REFERENCE OF SEQUENCE LISTING

The contents of the text file named “36770_535C01US_ST25.txt”, which was created on Oct. 12, 2017 and is 1.71 MB in size, are hereby incorporated by reference in their entireties.

### FIELD OF THE DISCLOSURE

The present invention relates to compositions and methods for diagnosing retinal neovascularization.

### BACKGROUND OF THE DISCLOSURE

Degenerative and vascular proliferative disorders of the retina, including photoreceptor/ganglion cell loss and ocular neovascularization (the most common cause of blindness in all age groups), is associated with retinopathy of prematurity in children, diabetic retinopathy in working age-adults and age-related macular degeneration in the elderly.

Prior to the invention described herein, the genetic makeup responsible for these diseases was unknown, rendering the identification and treatment of these diseases challenging. Thus, there is a pressing need in the art to identify diagnostic and treatment methods for retinal neovascularization.

### SUMMARY OF THE DISCLOSURE

The present invention relates to compositions and methods for the diagnosis of retinal vascular disease, e.g., retinal neovascularization. The invention also provides for isolated polynucleotides and corresponding polypeptides utilized to diagnose retinal vascular disease. The subject is preferably a mammal in need of such diagnosis/treatment, e.g., a subject with retinal vascular disease or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

A method of diagnosing retinal vascular disease in a subject is carried out by providing a test sample from a subject. For example, the test sample is obtained from a biological fluid selected from the group consisting of blood, vitreous humor, and aqueous humor. The retinal vascular disease is selected from the group consisting of diabetic retinopathy, age related macular degeneration, and retinopathy of prematurity. A genetic mutation associated with a region of human chromosome 3 or mouse chromosome 9 is detected in the subject. The sequence of Homo sapiens chromosome 3 is provided in GenBank Accession No. CM000665.1 (GI: NC_000003.11), incorporated herein by reference. The genetic mutation may be in a gene or regulatory region of a gene located in human chromosome 3 or mouse chromosome 9.

For example, the genetic mutation comprises a genetic mutation in Kyphoscoliosis peptidase (Ky) or a regulatory region thereof, centrosomal protein 63 (Cep63) or a regulatory region thereof, anaphase promoting complex subunit 13 (Anapc13) or a regulatory region thereof, angiomotin-like 2 (Amotl2) or a regulatory region thereof, Gm5627 or a regulatory region thereof, receptor-like tyrosine kinase (Ryk) or a regulatory region thereof, solute carrier organic anion transporter family or a regulatory region thereof, member 2a1 (Slco2a1) or a regulatory region thereof, member RAS oncogene family (Rab6b) or a regulatory region thereof, signal recognition particle receptor, B subunit (Srprb) or a regulatory region thereof, transferrin (trf) or a regulatory region thereof, 1300017J0RIK or a regulatory region thereof, topoisomerase (DNA) II binding protein 1 (Topbp1) or a regulatory region thereof, or carnitine deficiency-associated gene expressed in ventricle 3 (Cdv3) or a regulatory region thereof. In some embodiments, the genes are regulated by regulatory regions (or sequences) that are located on a different chromosome (i.e., the regulatory region is not located on human chromosome 3 or mouse chromosome 9). In other embodiments, the genes are regulated by regulatory regions (or sequences) that are located on the same chromosome, and at least 500 bp, 1,000 bp, 5,000 bp, 10,000 bp, 15,000 bp, 20,000 bp, 25,000 bp, 50,000 bp, or 100,000 bp from the genes (i.e., the transcription start or termination site) described herein. In some embodiments, the regulatory regions are directly flanking the gene that is being regulated; for example, the regulatory region is directly upstream of the transcription start site or directly downstream of the transcription start site.

Preferably, the genetic mutation is associated with a region of human chromosome 3. In some cases, the genetic mutation is located in at least one of Ky cDNA provided in GenBank Accession No. BC024014.1 (GI: 21955431) (SEQ ID NO: 15), Cep63 cDNA sequence provided in GenBank Accession No. BC014050.2 (GI:33878420) (SEQ ID NO: 16), Anapc13 cDNA sequence provided in GenBank Accession No. BC005398.1 (GI:13529286) (SEQ ID NO: 17), AmotL2 cDNA sequence provided in GenBank Accession No. BC011454.1 (GI:15079242) (SEQ ID NO: 18), Ryk cDNA sequence provided in GenBank Accession No. NM_001005861 (XM_001130617) (SEQ ID NO: 19), Slco2a1 cDNA sequence provided in GenBank Accession No. BC041140.1 (GI:26996626) (SEQ ID NO: 20), Srprb cDNA sequence provided in GenBank Accession No. BC063001.1 (GI:38649414) (SEQ ID NO: 21), Trf mRNA sequence provided in GenBank Accession No. NM_001063.3 (GI:257196241) (SEQ ID NO: 22), Topbp1 cDNA sequence provided in GenBank Accession No. BC151237.1 (GI:153217452) (SEQ ID NO: 23), Cdv3 cDNA sequence provided in GenBank Accession No. BC007338.2 (GI:33873572) (SEQ ID NO: 24), or Rab6b cDNA sequence provided in GenBank Accession No. BC078662.1 (GI:50927455) (SEQ ID NO: 25), or regulatory regions thereof.

Preferably, the genetic mutation is associated with a region of human chromosome 3. In some cases, the genetic mutation is located in at least one of Ky DNA provided in GenBank Accession No. NC_000003.11 (GI224589815:c134378285-134311000) (SEQ ID NO: 302), Cep63 DNA sequence provided in GenBank Accession No. NC_000003.11 (GI: 224589815:134191182-134307246) (SEQ ID NO: 303), Anapc13 DNA sequence provided in GenBank Accession No. NC_000003.11 (GI: 224589815:c134206112-134195297) (SEQ ID NO: 304), AmotL2 DNA sequence provided in GenBank Accession No. NC_000003.11 (GI: |224589815:c134097340-134071166) (SEQ ID NO: 305), Ryk DNA sequence provided in GenBank Accession No. NC_000003.11 (GI 224589815:c133983626-133861936) (SEQ ID NO: 306), Slco2a1 DNA sequence provided in GenBank Accession No. NC_000003.11 (GI: 224589815:c133763526-133636932) (SEQ ID NO: 307), Srprb DNA sequence provided in GenBank Accession No. NC_000003.11 (GI: 224589815:133497257-133545954) (SEQ ID NO: 308), Trf DNAsequence provided in GenBank Accession No. NC_000003.11 (GI: 224589820:73915262-73965820) (SEQ ID NO: 309), Topbp1 DNA sequence provided in GenBank Accession No. NC_000003.11 (GI: 224589815:c133389958-133310251) (SEQ ID NO: 310), Cdv3 DNA sequence provided in GenBank Accession No. NC_000003.11 (GI: 224589815:133289930-133311620) (SEQ ID NO: 311), or Rab6b DNA sequence provided in GenBank Accession No. NC_000003.11 (GI: 224589815:c133625432-133532336) (SEQ ID NO: 312), or regulatory regions thereof.

Alternatively, the genetic mutation is associated with a 1.0 Mb region of mouse chromosome 9 between the position 102,450,352 and 103,338,569 (i.e., SEQ ID NO: 14) as stated on mouse genome GRCm38.p1, including at least 50 Kb (kilobases) flanking said region, or the corresponding human sequence thereof. Also provided herein is the genetic mutation associated with human chromosome 3 between the position 132,757,235 and 134,370,478, including 50,000 base pairs flanking said region. For example, the genetic mutation is located in at least one of SEQ ID NOs: 1-13, or the corresponding human sequence thereof, for example SEQ ID NOs: 15-25 or SEQ ID NOs: 302-312.

As described herein, “corresponding sequence” refers to homologous sequences, such as homologous sequences found in a different species. Identification of homologous sequences is based on the similarity of the sequence, chromosomal synteny, or both. The term homology is used to indicate a likeness of structure and conservation of biological function. Calculations of nucleic acid or amino acid sequence identity provide a convenient method of identifying corresponding, homologous, or related genes, herein “homologs”. Such homologs may be members of a gene family or multiple genomic sequences present in the same genome, or may be corresponding genes or genomic sequences from different species (i.e., mouse and human). Chromosomal synteny may be used to further distinguish between homologous genes when there is sufficient evolutionary conservation between homologous genes when there is sufficient evolutionary conservation between the genomes that are being compared (i.e., between mammalian species). A “syntenic homolog” has both sequence identity to the reference gene, and has the corresponding chromosomal location in relation to the closely linked gene. Syntenic homologs have a high probability of sharing spatial and temporal localization of gene expression, and of encoding proteins that fill equivalent roles.

Homologous sequences can have substantial sequence similarity, i.e., homologous sequences between mouse and human. For example, homologous sequences may be at least 50%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity with the nucleotide or amino acid sequence with the subject sequence. The sequences, or fragments thereof, with genetic mutations that can be used for diagnosing a retinal vascular disease are preferably about 50%, about 55%, about 60%, about 65%, about 70%, about 75%, about 80%, about 85%, about 90%, about 91%, about 92%, about 93%, about 94%, about 95%, about 96%, about 97%, about 98%, about 99% identical to the sequences provided herein; and any range derivable therein, such as, for example, from about 70% to about 80%, and more preferably from about 81% to about 90%; or even more preferably, from about 91% to about 99% identity. Algorithms for sequence analysis are known in the art, such as BLAST, described in Altschul et al. (1990) J Mol Biol 215:403-10. Other methods for sequence analysis include pairwise alignment by dot-matrix methods, or structural alignment by distance matrix alignment or sequential structure alignment program (SSAP).

The genetic mutation detected is a point mutation, or a single base substitution where a single base nucleotide is replaced with a different nucleotide. In some embodiments, the genetic mutation is a transversion from adenine (A) to thymine (T), or thymine (T) to adenine (A). In other embodiments, the genetic mutation is A or T to G or C. The mutation can be a missense, nonsense, or splice mutation. Other mutations include additions, deletions, or chromosomal arrangements.

Any of the genetic mutations described herein can result in a in a gain-of-function mutation or a loss-of-function mutation in the gene. In a gain-of-function mutation, the gene product, or polypeptide encoded from the nucleic acid with the genetic mutation, gains a new and abnormal function. Gain-of-function mutations can be determined by assessing polypeptide activity. In a loss-of-function mutation, there may be no gene product or encoded polypeptide, or the encoded polypeptide has less or no function.

Also provided are methods of detecting a genetic mutation associated with a region of human chromosome 3 or mouse chromosome 9 in a subject. The region of human chromosome 3 or mouse chromosome 9 includes the genes described herein, and the regulatory regions thereof. In some embodiments, nucleic acids or polypeptides are isolated from a sample from the subject for detection of the genetic mutation. The nucleic acid may be DNA or RNA. Methods for isolating DNA and/or RNA are well-known in the art. Polypeptides can be isolated using any suitable method known in the art. The detection of the mutation is performed by nucleic acid detection techniques known in the art, such as amplification based techniques, real-time PCR, Northern blotting, microarray, PCR-based sequencing, Sanger sequencing, or next-generation sequencing. Alternatively, the polypeptides encoded by the nucleic acids described herein are isolated by methods known in the art. For example, polypeptides are typically detected by contacting a sample with an antibody or other specifically binding reagent in an immunoassay, such as ELISA or western blotting. The presence or absence of a genetic mutation associated with a region of human chromosome 3 or mouse chromosome 9 in a subject is useful for diagnosing retinal vascular disease. The presence of the genetic mutation in a subject indicates the presence or increased risk or predisposition for developing the retinal vascular disease. The absence of the genetic mutation in a subject indicates the absence or reduced risk or predisposition for developing the retinal vascular disease.

Also provided herein are methods for selecting a therapeutic regimen for a subject. The presence of a genetic mutation associated with a region of human chromosome 3 or mouse chromosome 9 in a subject, or a gene associated with the region as described herein, indicates that the subject should receive a therapeutic regimen comprising, for example, an anti-vascularization agent, an anti-angiogenesis agent, anti-inflammatory agent, or surgical procedures including but not limited to panretinal photocoagulation, laser photocoagulation, vitrectomy, scleral buckling, or peripheral retinal ablation.

Also provided is an isolated polynucleotide comprising the nucleic acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. Also provided is an isolated polynucleotide comprising the nucleic acid sequence set forth in SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, or SEQ ID NO: 312. Also provided are isolated polynucleotides comprising the nucleic acid sequences at least 50%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, or SEQ ID NO: 312. In some cases, the polynucleotide comprises a complementary deoxyribonucleic acid (cDNA) sequence or a messenger ribonucleic acid (mRNA) sequence.

Isolated polypeptides comprise an amino acid sequence encoded by the isolated polynucleotide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, or SEQ ID NO: 312.

An isolated polynucleotide comprising a functional region of SEQ ID NO: 14 or a fragment thereof is also provided.

The sequences of various regions of mouse chromosome 9 are provided herein. However, the invention also comprises polypeptides and nucleic acid fragments, so long as they exhibit the desired biological activity (i.e., result in the retinal vascularization phenotype) of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

Polynucleotides, polypeptides, antibodies, or other agents are purified and/or isolated. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques.

Similarly, “conservative amino acid substitutions,” in one or a few amino acids in an amino acid sequence are substituted with different amino acids with highly similar properties are also readily identified as being highly similar to a particular amino acid sequence, or to a particular nucleic acid sequence which encodes an amino acid. Such conservatively substituted variations of any particular sequence are a feature of the present invention. Individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. See, e.g., Creighton (1984) Proteins, W.H. Freeman and Company, incorporated herein by reference.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide.

Although the phrase “nucleic acid molecule” primarily refers to the physical nucleic acid and the phrase “nucleic acid sequence” refers to the linear list of nucleotides of the nucleic acid molecule, the two phrases can be used interchangeably.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a schematic of cellular components and molecular cascades during formation of the retinal superficial vascular plexus.

FIG. 2 is a series of images showing clinical phenotype of Nrv1 retinas depicts cotton-wool spotting and larger, tortuous blood vessels. Top panels: Fundus photos of wild-type (Nrv1+/+, left) and affected (Nrv1+/−, right) retina at postnatal day 30 illustrating abnormal blood vessels (middle panel on the top) and cotton-wool spotting (right panel on the right). Bottom Panels: fluoroangiography of wild-type (Nrv1+/+, left) and affected (Nrv1+/−, right) retina at postnatal day 30 illustrating tortuous and larger blood vessels in the retina (middle panel on the bottom) and abnormal vessels protruding in the vitreous (right panel on the right).

FIG. 3 is a series of images showing that the Nrv1 retina exhibits tortuous vessels. Immunofluorescence analysis on flat-mount retinas from wild-type (Nrv1+/+, left) and affected (Nrv1+/−, right) mice with an antibody against alpha-smooth muscle actin (red), a marker for major blood vessels. The analysis shows the tortuosity of blood vessels in the affected retina when compared to wild-type retina.

FIG. 4 is a series of images depicting that the Nrv1 model shows neovascularization into the vitreous. Hematoxylin and eosin stained retinal sections from wild-type (Nrv1+/+, left) and affected (Nrv1+/−, right) mice showing abnormal blood vessels protruding from the retina into the vitreous in the affected animals.

FIG. 5 is a series of images showing that neovascularization in Nrv1 mouse comes out from the retina at p10 while the hyaloid vasculature regresses normally. FIG. 5A and FIG. 5B provide alternate views. Histological analysis performed at different postnatal time points (p8, p10, p14, p21) on wild-type (Nrv1+/+, left) and affected (Nrv1+/−, right) retina sections showing the regression of the hyaloid vasculature (markes by asterisks) in both wild-type and affected and the abnormal protrusion of retinal vessels into the vitreous starting from p10 (marked by arrows).

FIG. 6 is a series of images showing new blood vessels in the vitreous are surrounded by astrocytes. Immunofluorescence analysis on flat-mount retinas from 30 days old wild-type (Nrv1+/+, left) and affected (Nrv1+/−, right) mice with antibodies against GFAP (a marker for astrocytes, in red), COLIV (a marker for blood vessels, in green) and pan-laminin (a marker for the basement membranes, in pink). The analysis shows in the affected animals the presence of astrocytes around the blood vessel in the vitreous.

FIG. 7 is a series of images showing new blood vessels in the vitreous are surrounded by astrocytes. Immunofluorescence analysis on flat-mount retinas from 30 days old affected (Nrv1+/−) mice with antibodies against GFAP (a marker for astrocytes, in red), and pan-laminin (a marker for the basement membranes, in pink). The analysis shows the presence of astrocytes around the blood vessel in the vitreous (marked by the arrow).

FIG. 8 is a series of images showing that astrocytes guide aberrant vessels into the vitreous. Immunofluorescence analysis at different postnatal time points (p4, p6, p8, p10) on wild-type (Nrv1+/+, left) and affected (Nrv1+/−, right) retina sections with antibodies against GFAP (a marker for astrocytes, in red) and COLIV (a marker for blood vessels, in green). The analysis shows the presence of astrocytes (orange arrows) around the blood vessels in the primary plexus of both normal and affected mice and their abnormal presence in the vitreous of affected animals starting from p4. Nuclei are shown in blue.

FIG. 9 is a series of images showing that disorganization of astrocyte network leads to abnormal vascular topography. Immunofluorescence analysis on flat-mount retinas from wild-type (Nrv1+/+, left) and affected (Nrv1+/−, right) mice with antibodies against GFAP (a marker for astrocytes, in red) and COLIV (a marker for blood vessels, in green) showing the disruption of organization of astrocytes network and abnormal vascular topography in the affected mice.

FIG. 10 is a series of images showing that astrocyte topography is disorganized in the Nrv1 mouse. Confocal analysis on flat mount retinas from wild-type (Nrv1+/+, left) and affected (Nrv1+/−, right) mice analyzes as in FIG. 8 showing the disruption of organization and topography of astrocytes in affected mice.

FIG. 11 is a series of images showing that vascular density appear to be reduced in Nrv1 mutant retina. Movie showing the three layers of the retinal vasculature. The movie was recorded with a confocal microscope on flat mount retinas from wild-type (Nrv1+/+, left) and affected (Nrv1+/−, right) mice stained with antibodies against GFAP (a marker for astrocytes, in red) and COLIV (a marker for blood vessels, in green). The movie shows a reduction of vascular density in the deeper layers.

FIG. 12 is a schematic depicting genome-wide scan—the backcrossing strategy. Schematic representation of the backcrossing stategy used to generate recombinant for linkage analysis. A cross of C57BL/6J×CastEi/J was established to generate F1/N 1. Affected offspring were backcrossed to CastEi/J to generate recombinant N2 pups. Disease associated alleles are shown in red, non-disease associated alleles are shown in yellow.

FIG. 13 shows that genome-wide scan maps the retinal defects to Chromosome 9. Genome-wide scan was performed on pooled DNA samples by using 69SSLP markers. Markers were analyzed by electrophoresis. The Marker D9Mit346 (left panel) was linked to the disease showing heterozygosity for the affected samples and homozygosity for the non-diseased-associated CastEi/J allele in the unaffected samples. Marker D10Mit16 (right panel), as well as all the others, was unlinked showing heterozigosity for both affected and unaffected samples. Nrv1+/+: unaffected mice; Nrv1−/+ affected mice.

FIG. 14 is a schematic depicting that genetic finemapping narrows Nrv1 interval to 1.0 Mb. On the left, the physical map of chromosome 9 is shown with the genetic markers used for finemapping. Flanking markers are shown in red. On the right, all of the recombinants that help refine the Nrv1 interval are shown. Mapped recombinant events are depicted as colored chromosomes. The diseased-associated C57BL/6J chromosome carrying the Nrv1 mutation is shown in red while non-diseased associated CastEi/J chromosome is shown in black. The mouse identification number and its phenotype are given. Aff: affected.

FIG. 15 is next generation sequencing data used to identify the mutated gene into the 1.0 Mb.

### DETAILED DESCRIPTION OF THE INVENTION

Retinal vascular disease is a hallmark of many blinding disorders such as diabetic retinopathy (DR) (affects millions worldwide), age related macular degeneration (AMD) (affects 1 in 3 people over the age of 65) and childhood diseases such as retinopathy of prematurity (ROP). These diseases cause the formation of extra blood vessels and lead to debilitating vision loss and eventually blindness.

The present invention provides compositions and methods for diagnosing, treating and/or preventing ophthalmic or ocular disorders, diseases or conditions, such as retinal vascular disease. The subject is preferably a mammal in need of such treatment, e.g., a subject with diabetic retinopathy or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human. The terms “ophthalmic” and “ocular” are used interchangeably herein.

Specifically, the invention described herein characterizes a genetic mouse model that recapitulates the retinal vascular phenotype observed in diabetic retinopathy patients. This mouse strain, NMF128, carries a chemically (ENU)-induced mutation with a semi-dominant mode of inheritance. NMF128 was generated by ENU-induced mutagenesis at The Jackson Laboratory Neuroscience Mutagenesis Facility. The phenotype is characterized by early-onset retinal neovascularization and protusion of retinal vessels onto the vitreous body, a typically avascular portion of the eye. As described herein, the abnormal vitreal vessels exhibit tortuosity, a clinical indicator for abnormal vasculature, and originate from the retinal vasculature. In addition, the abnormal vitreal vessels in NMF128 eyes express a retinal neovascularization agent, vascular endothelial growth factor (VEGF), long after normal retinal vasculature development is complete. The mouse model described herein is a valuable tool for the study of pathology, progression and the genetic networks that are perturbed in retinal neovascularization diseases.

### Retina Structure

The vertebrate retina is a light sensitive tissue lining the inner surface of the eye. The optics of the eye create an image of the visual world on the retina, which serves much the same function as the film in a camera. Light striking the retina initiates a cascade of chemical and electrical events that ultimately trigger nerve impulses. These are sent to various visual centers of the brain through the fibers of the optic nerve. A number of changes can occur in the retina with age. For example, atherosclerotic buildup and leakage in the retinal arteries can lead to macular degeneration as well as reduction of peripheral vision. Rods and cones of the retina can become less sensitive over time, as they replenish their pigments more slowly. Progressively, all these effects can reduce vision, ultimately leading to partial or complete blindness. Retinal diseases such as age-related macular degeneration are difficult to treat, and current retinal treatments include invasive laser surgery to stop the leaking of blood vessels in the eye.

### Retina Pathology

Retinal diseases include proliferative diabetic retinopathy, age-related macular degeneration (AMD), retinopathy of prematurity, and retinal branch vein occlusion. Diabetic retinopathy (DR) remains the most common cause of vision impairment in working-age adults in the United States and Europe, and retinal neovascularization occurs in up to 20% of patients with diabetes. Pathological retinal neovascularization in patients with diabetes results from an imbalance of pro-angiogenic and anti-angiogenic factors. In addition to VEGF accumulation in eyes with diabetic retinal neovascularization, changes in numerous other cytokines, chemokines, adhesion molecules, vasoactive hormones and immune cells have been reported. Together, these changes constitute a complex inflammatory process that results in an aberrant wound-healing response. All retinal cell types, including neurons, glial, microglial and vascular cells, are believed to be affected by diabetes, resulting in a neurovascular disorder. The retinal neurovascular degeneration of diabetes includes neuronal and vascular cell apoptosis, and microglial and glial cell activation, which provides intraretinal sources of cytokines and chemokines (Gariano & Gardner, Nature 438, 960-966, 2005).

Clinically, diabetic retinopathy (DR) is classified into two stages: non-proliferative DR (NPDR) and proliferative DR (PDR). In NPDR, VEGF-A (which is known to act as a survival factor for endothelial cells, via induction of anti-apoptotic proteins) expression in elevated levels has been described in human and in experimental models of DR. VEGFRs are also upregulated in NPDR. VEGF-A may be increased initially in preclinical DR as a mechanism to maintain the integrity of the retinal vascular bed. In a later stage, high VEGF-A production in ischemic areas, where VEGFR-2 is upregulated, then leads to vascular leakage and neovascularization. In proliferative DR (PDR), intra-ocular neovascularization is likely caused by high levels of VEGF-A in the vitreous derived from widespread production of this factor by ischemic retina. Elevated levels of VEGF-A have been found in the aqueous humor and vitreous of patients with PDR (Witmer et al., Prog Ret Eye Res 22, 1-29, 2003).

Angiogenesis, the formation of new blood vessels from preexisting vasculature, is a major component in several retinal vascular diseases causing blindness, such as retinopathy of prematurity, proliferative diabetic retinopathy, and age-related macular degeneration described above. Diabetes mellitus is the main cause of new cases of adult blindness. Nearly all individuals with type 1 diabetes show some symptoms of diabetic retinopathy, usually ˜20 years after clinical recognition of diabetes; in the most advanced form of diabetic retinopathy, called proliferative diabetic retinopathy, new blood vessels grow uncontrollably on the retinal inner surface, causing hemorrhages and even retinal detachment. Surgical and laser photocoagulation treatments are only partially effective and may further damage the retinal tissue.

Prior to the invention described herein, the genetic makeup responsible for these diseases was unknown, rendering the identification and treatment of these diseases challenging. Accordingly, the invention is based on the characterization of the genetic model that recapitulates retinal vascular disease in humans.

### Detection of Genetic Mutations

Also provided herein are methods for detecting genetic mutations associated with the 1.0 Mb region identified herein. Nucleic acids or polypeptides are isolated or extracted from the sample using standard molecular biology techniques. The genetic mutations are then detected from the isolated nucleic acids and/or polypeptides as described below.

Detection of the genetic mutations described herein is performed preferably by techniques known in the art in one embodiment DNA sequencing reactions using polymerase chain reaction (PCR) are used to screen the sequences described herein in a sample from a subject. Methods for nucleotide sequencing of genetic or other biological material are known in the art.

The isolated nucleic acid sequences are optionally amplified before being sequenced. The term “amplified” defines the process of making multiple copies of the nucleic acid from a single or lower copy number of nucleic acid sequence molecule. The amplification of nucleic acid sequences is carried out in vitro by biochemical processes known to those of skill in the art such as PCR or by cloning and expansion.

One process of in vitro amplification, which is used according to this invention, is the polymerase chain reaction (PCR) illustratively described in U.S. Pat. Nos. 4,683,202 and 4,683,195 the contents of which are incorporated herein by reference. The term “polymerase chain reaction” refers to a process for amplifying a DNA base sequence using a heat-stable DNA polymerase and two oligonucleotide primers, one complementary to the (+)-strand at one end of the sequence to be amplified and the other complementary to the (−)-strand at the other end. Because the newly synthesized DNA strands subsequently serve as additional templates for the same primer sequences, successive rounds of primer annealing, strand elongation, and dissociation produce rapid and highly specific amplification of the desired sequence. Many polymerase chain processes are known to those of skill in the art and are operable in the process of the invention. For example, DNA is subjected to 30 to 35 cycles of amplification in a thermocycler as follows: 95° C. for 30 sec, 52 to 60° C. for 1 min, and 72° C. for 1 min, with a final extension step of 72° C. for 5 min. For another example, DNA is subjected to 35 polymerase chain reaction cycles in a thermocycler at a denaturing temperature of 95° C. for 30 sec, followed by varying annealing temperatures ranging from 54 to 58° C. for 1 min, an extension step at 70° C. for 1 min, with a final extension step at 70° C. for 5 min. The parameters of PCR cycling times and number of steps are dependent on the primer pair, their melting temperature, and other considerations known to those of skill in the art. It is appreciated that optimizing PCR parameters for various primer and/or probe sets is well within the skill of the art and is performed as mere routine optimization.

The amplification agent for PCR is optionally any compound or system that will function to accomplish the synthesis of primer extension products, including enzymes. Suitable enzymes for this purpose include, for example, E. coli DNA polymerase I, Taq polymerase, Klenow fragment of E. coli DNA polymerase I, T4 DNA polymerase, AmpliTaq Gold DNA Polymerase from Applied Biosystems, other available DNA polymerases, reverse transcriptase (preferably iScript RNase H+ reverse transcriptase), ligase, and other enzymes, including heat-stable enzymes (i.e., those enzymes that perform primer extension after being subjected to temperatures sufficiently elevated to cause denaturation). In a preferred embodiment, the enzyme is hot-start iTaq DNA polymerase from Bio-rad (Hercules, Calif.). Materials including enzymes operable to amplify target genes are optionally contained in a kit such as the Herculase PCR master mix (Stratagene, La Jolla, Calif). Suitable enzymes will facilitate combination of the nucleotides in the proper manner to form the primer extension products that are complementary to each mutant nucleotide strand. Generally, synthesis is initiated at the 3′-end of each primer and proceed in the 5′-direction along the template strand, until synthesis terminates, producing molecules of different lengths. There are optionally amplification agents, however, that initiate synthesis at the 5′-end and proceed in the other direction, using the same process as described above. In any event, the process of the invention is not to be limited to the embodiments of amplification described herein.

The primers for use in amplifying the nucleic acid sequences of Fzd-4 are optionally prepared using any suitable process, such as conventional phosphotriester and phosphodiester processes or automated embodiments thereof so long as the primers are capable of hybridizing to the nucleic acid sequences of interest. One process for synthesizing oligonucleotides on a modified solid support is described in U.S. Pat. No. 4,458,066, the contents of which are incorporated herein by reference. The exact length of primer will depend on many factors, including temperature, buffer, and nucleotide composition. The primer generally should prime the synthesis of extension products in the presence of the inducing agent for amplification.

Design of primers for specific amplification of the genes or sequences described herein is similarly performed by methods known in the art. Preferably, primers are designed to flank a mutational site of interest. Preferably, the primers are complementary to genetic sequences described herein. It is appreciated that the complement of the aforementioned primer and probe sequences are similarly suitable for use in the instant invention. It is further appreciated that oligonucleotide sequences that hybridize with the inventive primer and probes are also similarly suitable. Any of the primers and/or probes comprising SEQ ID NOS: 26-301 can be used in the present invention. Examples of suitable primers and/or probes can be found in Table 1.

Standard considerations of primer designs are taken into account when designing primers such as melting temperature, primer length, GC content, propensity to form secondary structure, identity of end nucleotides, and others known in the art. It is appreciated that modifications of primers of SEQ ID NOs: 26-301 are similarly operable.

Those of ordinary skill in the art will know of various amplification processes that are also used to increase the copy number of target nucleic acid sequence. The nucleic acid sequences detected in the process of the invention are optionally further evaluated, detected, cloned, sequenced, and the like, either in solution or after binding to a solid support, by any process usually applied to the detection of a specific nucleic acid sequence such as another polymerase chain reaction, oligomer restriction (Saiki et al., BioTechnology 3:1008 1012 (1985)), allele-specific oligonucleotide (ASO) probe analysis (Conner et al., PNAS 80: 278 (1983)), oligonucleotide ligation assays (OLAs) (Landegren et al., Science 241:1077 (1988)), RNase Protection Assay and the like. Molecular techniques for DNA analysis have been reviewed (Landegren et al., Science 242:229 237 (1988)). Following DNA amplification, the reaction product is optionally detected by Southern blot analysis, with or without using radioactive probes. In such a process, for example, a small sample of DNA containing the nucleic acid sequence obtained from the tissue or subject is amplified, and analyzed via a Southern blotting technique. The use of non-radioactive probes or labels is facilitated by the high level of the amplified signal. In one embodiment of the invention, one nucleoside triphosphate is radioactively labeled, thereby allowing direct visualization of the amplification product by autoradiography. In another embodiment, amplification primers are fluorescently labeled and run through an electrophoresis system. Visualization of amplified products is by laser detection followed by computer assisted graphic display, without a radioactive signal.

Other methods of detecting amplified oligonucleotide illustratively include gel electrophoresis, mass spectrometry, liquid chromatography, fluorescence, luminescence, gel mobility shift assay, fluorescence resonance energy transfer; nucleotide sequencing, enzyme-linked immunoadsorbent assay, affinity chromatography, chromatography, immunoenzymatic methods (Ortiz, A and Ritter, E, Nucleic Acids Res., 1996; 24:3280-3281), streptavidin-conjugated enzymes, DNA branch migration (Lishanski, et al., Nucleic Acids Res., 2000; 28(9):e42), enzyme digestion (U.S. Pat. No. 5,580,730), colorimetric methods (Lee, K., Biotechnology Letters, 2003; 25:1739-1742), or combinations thereof.

In one embodiment real-time PCR (RT-PCR) is used to detect the presence or absence of a mutation. Methods of RT-PCR are known in the art such as that used in the TaqMan system. Illustratively, a probe complementary to the mutation of interest is used to detect the presence or absence of a mutation. A probe is preferably hybridized to a region of DNA that has a target mutation. Preferably, a probe differentially hybridizes to a region with a mutation and is selective for that mutation. Illustratively, a probe will have greater hybridization with a site that contains a mutation than with the site in the absence of the mutation. Greater hybridization is illustratively determined by differences in melting temperature between complete hybridization and incomplete hybridization.

Optionally, other detection systems using RT-PCR are operable herein. Illustratively, allele specific PCR with blocking reagent (ASB-PCR) is used as a method of detection in the present invention. Methods of ASB-PCR are found in Morlan, J, et al., 2009; PUS ONE 4(2): e4584, doi:10.1371/journal.pone.0004584, the contents of which are incorporated herein by reference as applicable to the subject invention. Other methods such as that described in U.S. Application Publication No: 2008/0213756, the entire contents of which are incorporated herein by reference, are similarly operable.

In an alternative method the sequence of mRNA is detected. This process preferably uses reverse transcription PCR to synthesize cDNA from RNA collected from a subject. The sequence of the synthesized cDNA is then obtained by PCR and sequencing methods as known in the art or as described herein and determination of the presence or absence of a mutation is performed.

In a preferred embodiment a probe such as that used for RT-PCR is labeled. The term “labeled” with regard to the probe is intended to encompass direct labeling of the probe by coupling (i.e., physically linking) a detectable substance to the probe, as well as indirect labeling of the probe by reactivity with another reagent that is directly labeled. Examples of indirect labeling include detection of a probe using a fluorescently labeled antibody and end-labeling or centrally labeling of a DNA probe with biotin such that it is detected with fluorescently labeled streptavidin.

The detection processes of the invention are optionally used to detect RNA (particularly mRNA) or genomic nucleic acid in a sample in vitro as well as in vivo. For example, in vitro techniques for detection of nucleic acid include northern hybridizations, in situ hybridizations, RT-PCR, reverse transcription PCR, and DNase protection.

The size of the primers used to amplify a portion of the nucleic acid sequences described herein is optionally at least 5, and often 10, 15, 20, 25, or 30 nucleotides in length. Preferably, the GC ratio is above 30%, 35%, 40%, 45%, 50%, 55%, or 60% so as to prevent hair-pin structure on the primer. Furthermore, the amplicon is preferably of sufficient length to be detected by standard molecular biology methodologies. The forward primer is preferably shorter than the reverse primer. Techniques for modifying the Tₘ of either primer are operable herein. An illustrative forward or reverse primer contains LNA-dA and LNA-dT (Glen Research Corporation) so as to match Tₘ with a corresponding alternate primer.

An inventive process uses a polymerization reaction which employs a nucleic acid polymerizing enzyme, illustratively a DNA polymerase, RNA polymerase, reverse transcriptase, or mixtures thereof. It is further appreciated that accessory proteins or molecules are present to form the replication machinery. In a preferred embodiment the polymerizing enzyme is a thermostable polymerase or thermodegradable polymerase. Use of thermostable polymerases is well known in the art such as Taq polymerase available from Invitrogen Corporation (Carlsbad, Calif.). Thermostable polymerases allow a polymerization reaction to be initiated or shut down by changing the temperature or other condition in the reaction mixture without destroying activity of the polymerase.

In a further embodiment detection of PCR products is achieved by mass spectrometry. Mass spectrometry has several advantages over real-time PCR systems in that it is used to simultaneously detect the presence of Fzd-4 and decipher imitations in target nucleic acid sequences allowing identification of single or multiple mutations simultaneously. Further, mass spectrometers are prevalent in the clinical laboratory. Similar to fluorescence based detection systems, mass spectrometry is capable of simultaneously detecting multiple amplification products for a multiplexed and controlled approach to accurately quantifying components of biological or environmental samples.

Many mass spectrometry platforms are suitable for use in the instant invention illustratively including matrix assisted laser desorption ionization time of flight mass spectrometry (MALDI), electro spray mass spectrometry, electrospray ionization-Fourier transform ion cyclotron resonance mass spectrometry (ESI-FTICR), multi-stage mass spectrometry fragmentation analysis (MS/MS), mass spectrometry coupled with liquid chromatography such as high performance liquid chromatography mass spectrometry (HPLC) and ultra performance liquid chromatography isotope dilution tandem mass spectrometry (HPLC-ID/MS/MS), and variations thereof.

It is appreciated that numerous other detection processes are similarly suitable for measuring an amplification product by detecting a detection signal. Illustrative examples include, but are not limited to, liquid chromatography, mass spectrometry, liquid chromatography/mass spectrometry, static fluorescence, dynamic fluorescence, high performance liquid chromatography, ultra-high performance liquid chromatography, enzyme-linked immunoadsorbent assay, real-time PCR, gel electrophoresis, or combinations thereof.

A polypeptide, or a genetic mutation thereof, can be detected in any suitable manner, but is typically detected by contacting a sample from the subject with a reagent that specifically recognizes or binds the polypeptide or mutated polypeptide and then detecting the presence or absence of a reaction product. For example the reagent is an antibody. The antibody may be monoclonal, polyclonal, chimeric, or a fragment of the foregoing, as discussed in detail above, and the step of detecting the reaction product may be carried out with any suitable immunoassay. The sample from the subject is typically a biological fluid as described above, and may be the same sample of biological fluid used to conduct the method described above.

Immunoassays carried out in accordance with the present invention are used to detect the polypeptides, or genetic mutations thereof, as described herein. Examples of suitable immunoassays are immunoblotting, immunofluorescence methods, immunoprecipitation, chemiluminescence methods, electrochemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.”

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

For polypeptides known to have enzymatic activity, the activities can be determined in vitro using enzyme assays known in the art. Such assays include, without limitation, kinase assays, phosphatase assays, and reductase assays, among many others. Modulation of the kinetics of enzyme activities can be determined by measuring the rate constant K_{M} using known algorithms, such as the Hill plot, Michaelis-Menten equation, linear regression plots such as Lineweaver-Burk analysis, and Scatchard plot. A genetic mutation in the nucleic acid encoding the polypeptide or a genetic mutation in the regulatory region of the gene encoding the polypeptide can result in a gain-in-function (i.e., increase in enzymatic activity or different function) or a loss-of-function (i.e., decrease or loss in enzymatic activity).

In some cases, one or more steps of the methods of the invention are performed on a computer. For example, the detecting, measuring, calculating, and assigning steps are performed on a computer. In some embodiments, the invention provides an article of manufacture containing computer executable instructions stored on a non-transitory computer readable media, which, when executed by a computer, causes the computer to perform operations comprising the detecting, measuring, calculating, and/or assigning steps of the methods described herein. In some embodiments, the computer further generates a report indicating the diagnosis of the subject, where the report indicates whether the subject comprises a retinal vascular disease.

In some embodiments, the invention provides a non-transitory computer program product storing instructions, which when executed by at least one data processor of at least one computing system, implement a method described herein (e.g., a detecting, measuring, calculating, and assigning step of a method described herein). In other embodiments, the invention provides a computer-implemented method that performs the detecting, measuring, calculating, and/or assigning steps of the methods described herein. In some cases, the invention provides a non-transitory computer readable storage medium containing executable instructions to perform the detecting, measuring, calculating, and/or assigning steps of the methods described herein. In other cases, the invention features a system containing at least one data processor and memory storing instructions, which when executed by the at least one data processor, causes the at least one data processor to perform operations including the detecting, measuring, calculating, and/or assigning steps of the methods described herein.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying information to the user and a keyboard and a pointing device (e.g., a mouse or a trackball) by which the user may provide input to the computer. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

As described in detail below, the mouse model, Nrv1 (new retinal vessels 1), shows abnormal production of blood vessels in the retina and vitreous. These blood vessels are disorganized and clumped. A disorganized astrocytes network underlines the abnormal vascular topography. The abnormal blood vessels into the vitreous originate from retinal vasculature and are preceded by the uncommon presence of astrocytes into the vitreous. The data described herein indicate misguidance defects due to the astrocytes.

As described in detail below, linkage analyses mapped the vascular disorder to a 1 Mb region on the chromosome 9. Sequencing this region identifies the genetic mutations responsible for this disease. As described herein, characterization of this mouse model and its genetic cause assists in the development of diagnostic and viable treatment methods that prevent, attenuate, and/or cure these diseases.

### Example 1: Genetic Model of Retinal Neovascularization (Nrv1)

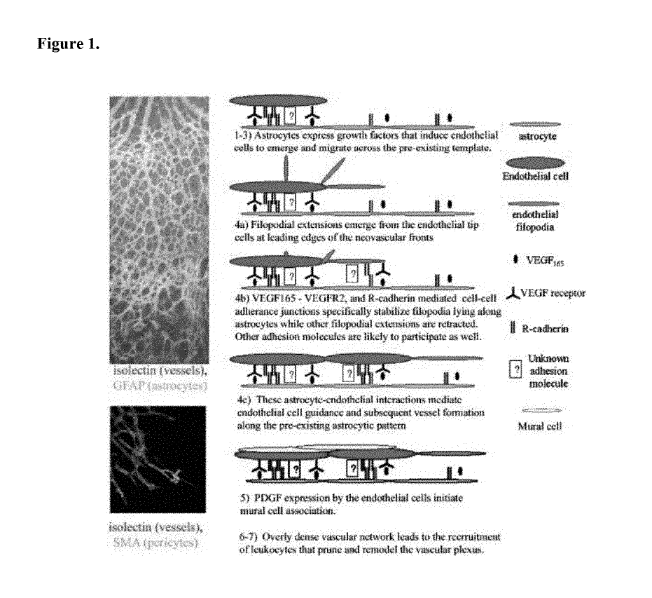

The retinal vasculature includes the superficial vascular plexus, the intermediate vascular plexus, and the deep vascular plexus. The cellular components and molecular cascades during formation of the retinal superficial vascular plexus are described in Dorrel M., 2006 Progress in Retinal and Eye Research (FIG. 1).

### Mechanisms of Retinal Vascular Defects and Diseases

Lack of hyaloid vessel regression: Persistent fetal vasculature syndrome (Norrin, Lama1). Lack of vascularization into retina: Persistent fetal vasculature syndrome (Lrp5). Neovascularization into vitreous: Proliferative diabetic retinopathy and retinopathy of prematurity (Nrv1).

The following are genetic approaches to generate mouse models: 1) Gene-driven approach (reverse genetics): gene/mutation→function/phenotype; and 2) Phenotype-driven approach (forward genetics): function/phenotype→gene/mutation.

As described herein, ENU Mutagenesis provides a way forward to understand gene function. The potency of this approach is 12 times that of radiation, and 200 times that of spontaneous mutation. This can provide single nucleotide substitution, 70-85% A-T to T-A or A-T to GC, 70% nonsynonymous change (65% missense and the remaining are nonsense or splice mutations). The chemical structure of N-Ethyl-N-nitrosurea is provided below.

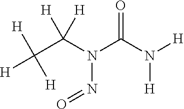

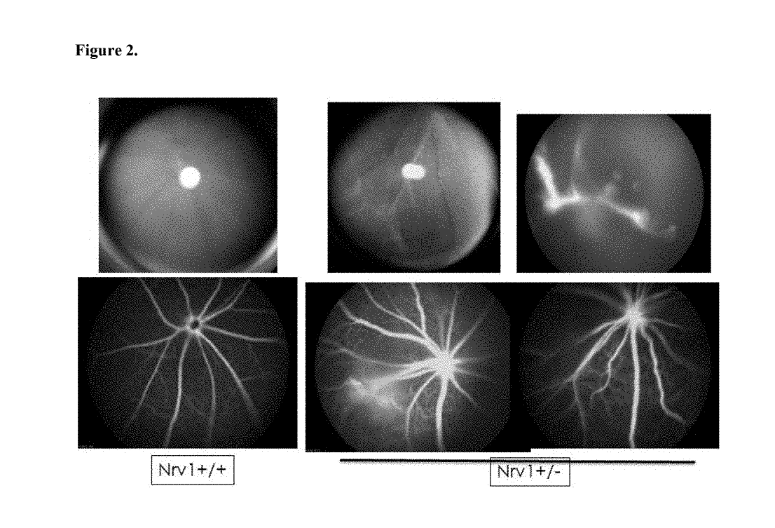

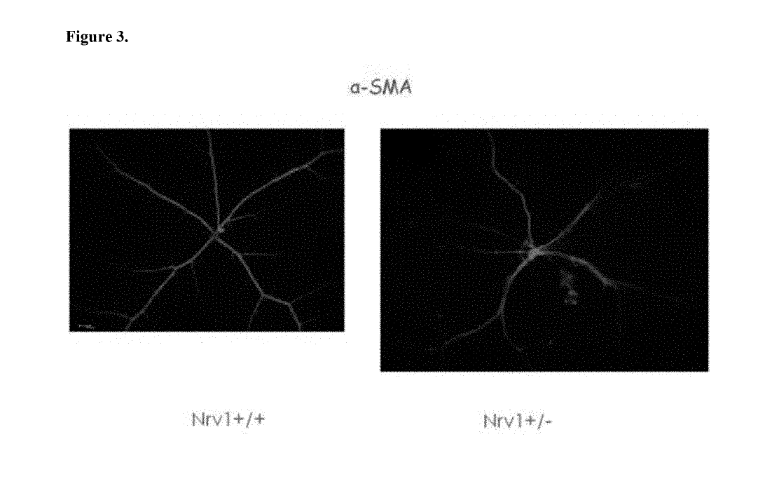

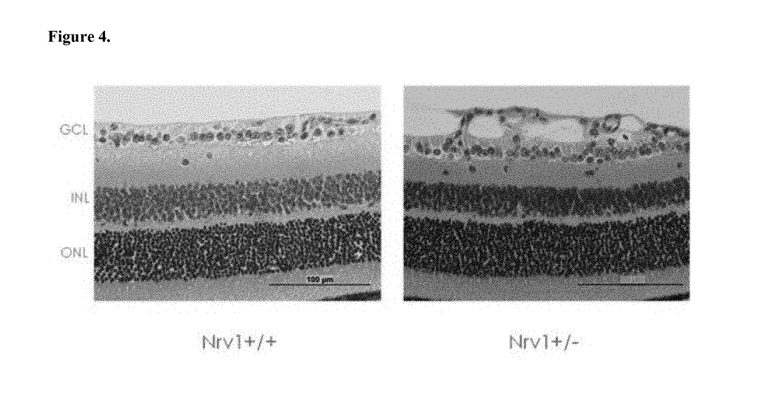

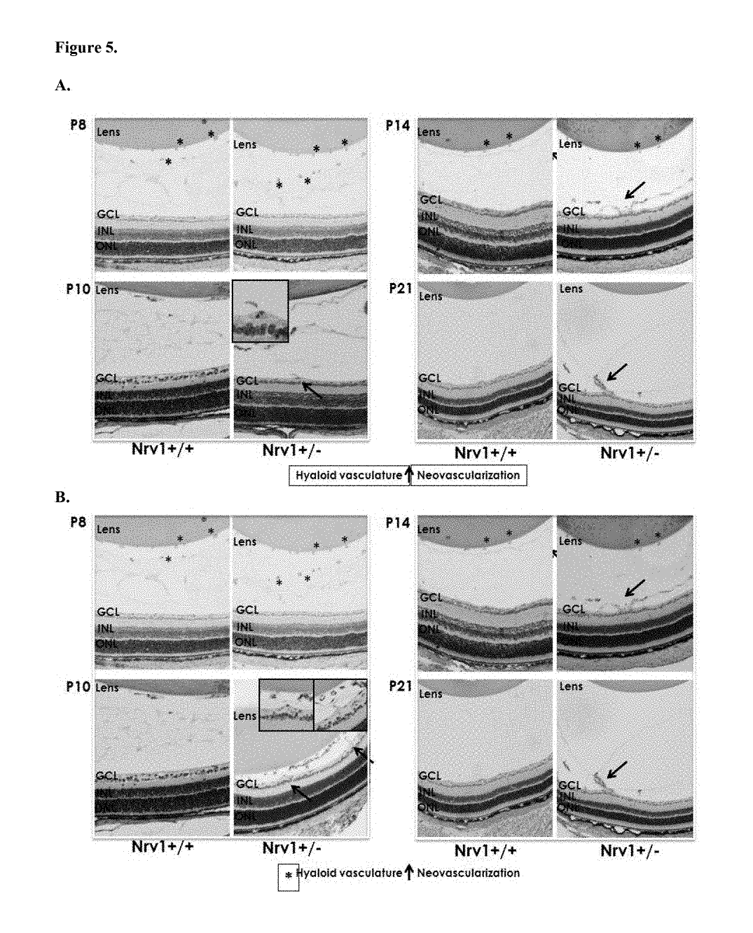

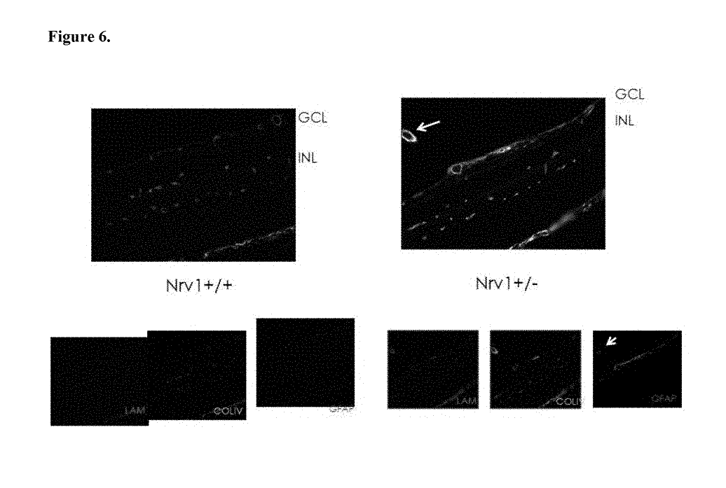

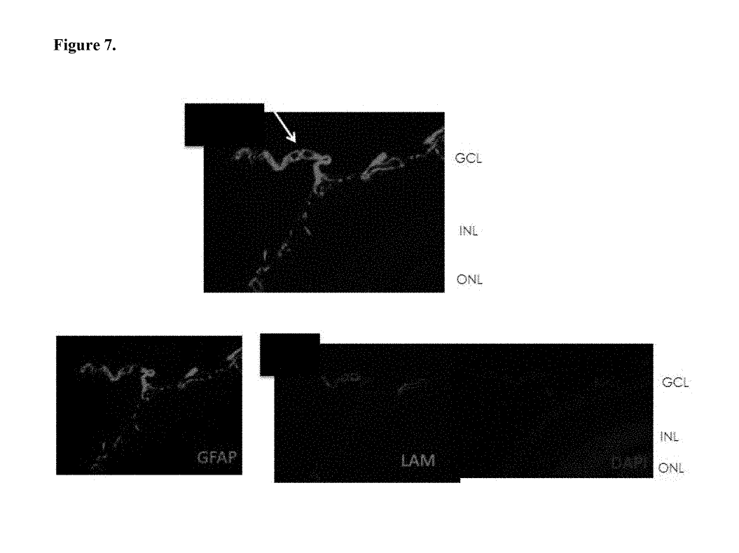

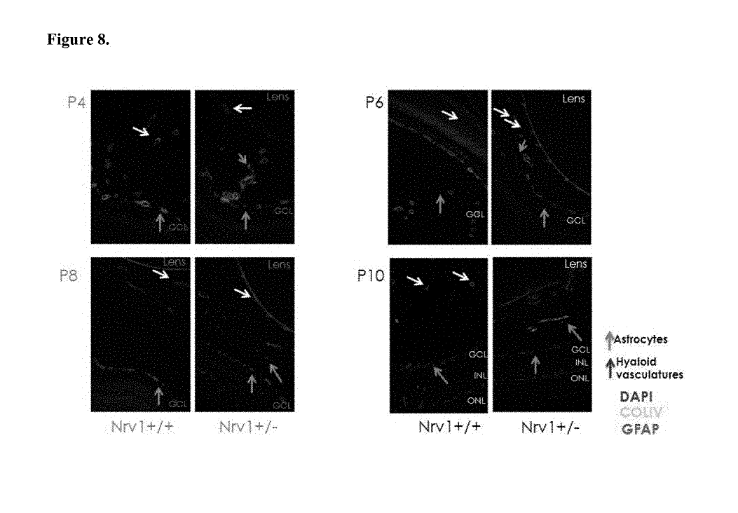

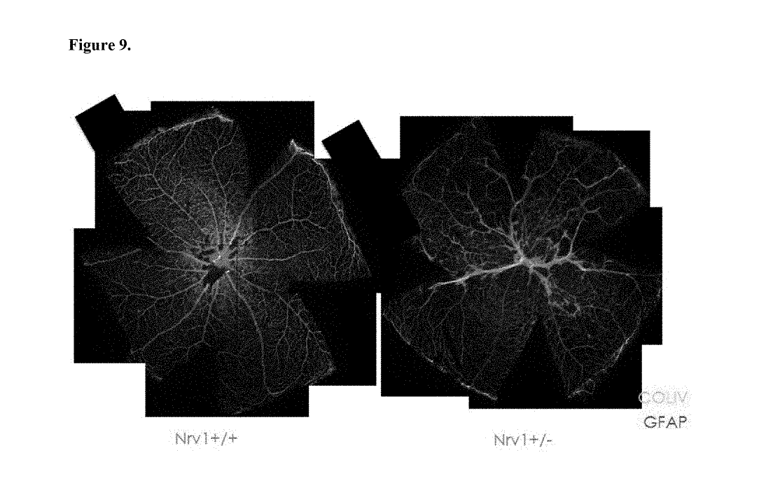

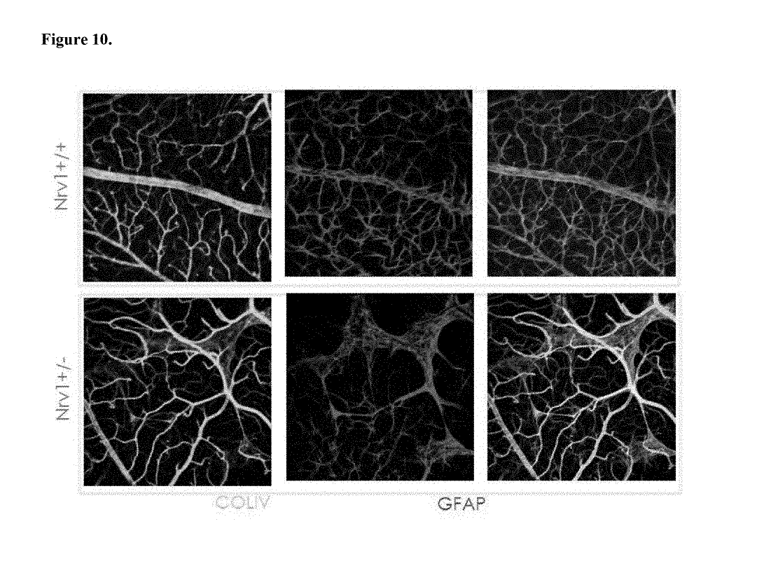

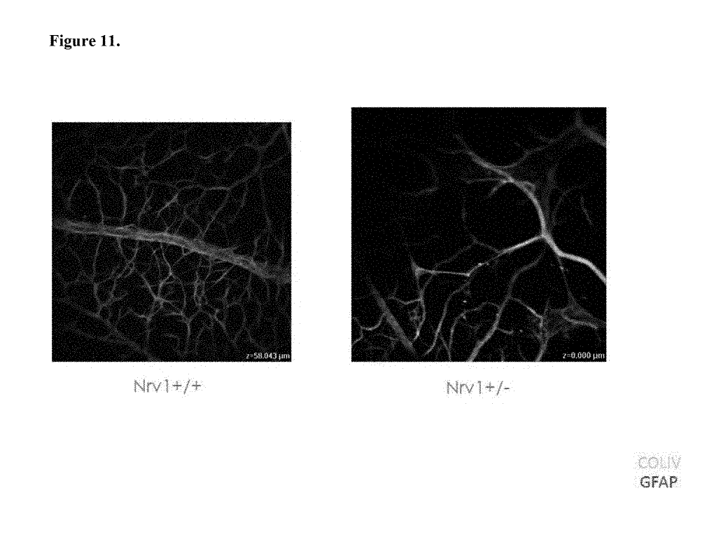

Described in detail below is phenotypic and histological characterization of the Nrv1 (new retinal vessels 1) mouse. FIG. 2 is a series of images showing clinical phenotype of Nrv1 retinas depicts cotton-wool spotting and larger, tortuosus blood vessels. FIG. 3 is a series of images showing that the Nrv1 retina exhibits tortuosuos vessels. FIG. 4 is a series of images depicting that the Nrv1 model shows neovascularization into the vitreous. FIG. 5 is a series of images showing that neovascularization in Nrv1 mouse comes out from the retina at p10 while the hyaloid vasculature regresses normally. FIG. 5A and FIG. 5B provide alternate views. FIG. 6 is a series of images showing new blood vessels in the vitreous are surrounded by astrocytes. FIG. 7 is a series of images showing new blood vessels in the vitreous are surrounded by astrocytes. FIG. 8 is a series of images showing that astrocytes guide aberrant vessels into the vitreous. FIG. 9 is a series of images showing that disorganization of astrocyte network leads to abnormal vascular topography. FIG. 10 is a series of images showing that astrocyte topography is disorganized in the Nrv1 mouse. FIG. 11 is a series of images showing that vascular density appear to be reduced in Nrv1 mutant retina.

In summary, the Nrv1 mouse phenotype has the following characteristics: 1) tortuous blood vessels; 2) larger major blood vessels; 3) abnormal vessels protruding into the vitreous; 4) reduced density of vessels in the inner retina; and 5) disruption of organization and topography of the astrocyte network.

As described herein, apoptosis/proliferation during vascular development is examined. Vascular development is also tracked on flat mount retina.

### Example 2: Mapping and Candidate Gene Identification of Nrv1

Mice are commonly used as models of human retinal disease in order to understand the genetic basis of disease, and to develop treatments for these diseases. Prior to the invention described herein, due to the complexity of the retina and secondary effects in systemic circulatory systems, genetic manipulation leading to retinal neovascularization has proven difficult. Specifically, creating mouse models of proliferative diabetic retinopathy and ischemic retinopathy using other methods including inducing diabetes or laser-induced vascular occlusions failed to produce a real disease phenotype. The mouse model of oxygen-induced retinopathy was the most common animal model for retinal angiogenesis studies. In this model for pathological neovascularization of the retina, pathology was driven by manipulating the concentration of oxygen that the mice breathe. However, this model was a transient model, in which the retinopathy was difficult to maintain and eventually regressed. Thus, prior to the invention, there was a pressing need for a genetic mouse model to examine retinal neovascularization. The NMF128 mouse mutant (Nrv1) described herein is a clinically relevant animal model for retinal neovascularization-related diseases, specifically for proliferative diabetic retinopathy.

The mouse model described herein, NMF128, develops a vascular anomaly phenotype that closely resembles human retinal vascular diseases such as proliferative diabetic retinopathy, ischemic retinopathy from vascular occlusions and systemic blood dyscrasia and retinopathy of prematurity. The NMF128 mouse model described herein provides a unique opportunity for the study of disorders associated with retinal neovascularization. The mouse model described herein is a valuable tool for the study of pathology, progression and the genetic networks that are perturbed in retinal neovascularization diseases. In addition, as described herein, this model is used to test genes for therapeutic value in the amelioration of disease with minimal negative consequences.

A genome-wide scan on pooled DNA samples was performed to identify the chromosome carrying the causative mutation. Genetic finemapping was then utilized to reduce the genomic interval. Linkage analyses mapped the vascular disorder to a 1 Mb region on the chromosome 9. Specifically, this disorder was genetically mapped to a 1.0 megabase (Mb) region on chromosome 9 containing 14 genes. Specifically, a 1 Mb region on chromosome 9 between the position 102,450,352 and 103,338,569 as stated on mouse genome GRCm38.p1 was identified as responsible for carrying the mutated gene. Genes in this region include Ky, Cep63, Anapc13, Amotl2, Gm5627, Ryk, Slco2a1, Rab6b, Srprb, Trf, 1300017J0RIK, Topbp1, and Cdv3. The homologous genes have been identified in humans.

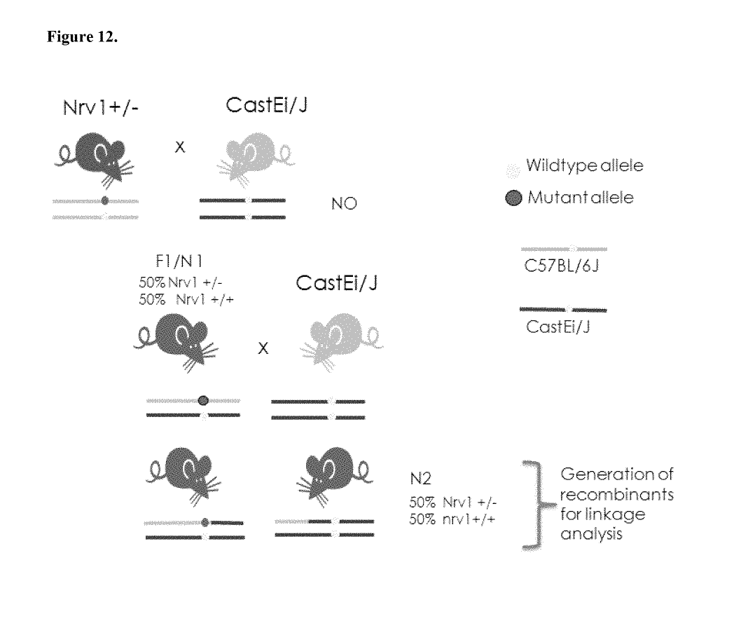

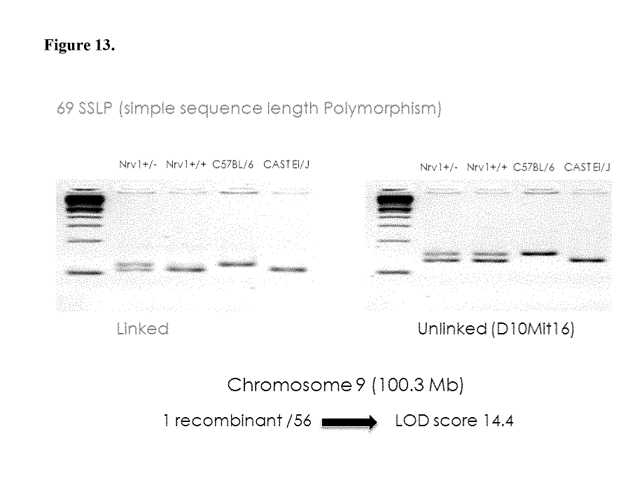

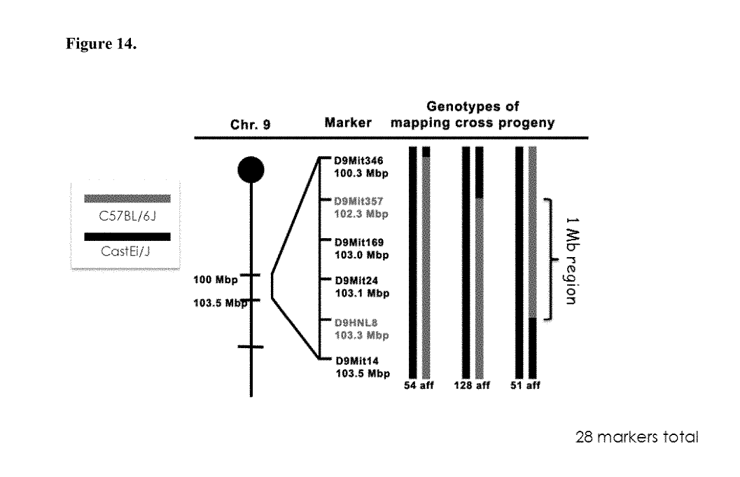

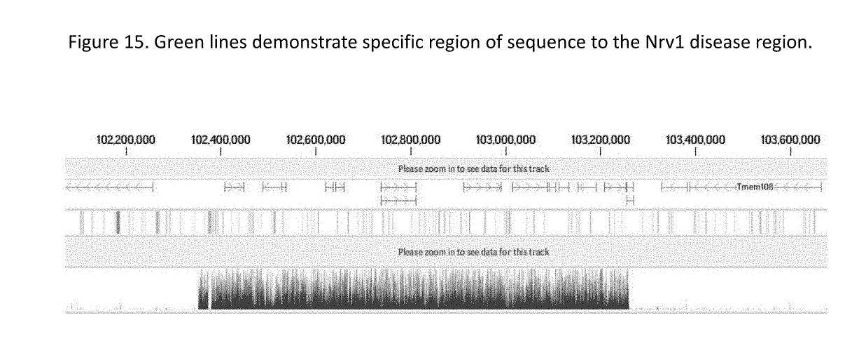

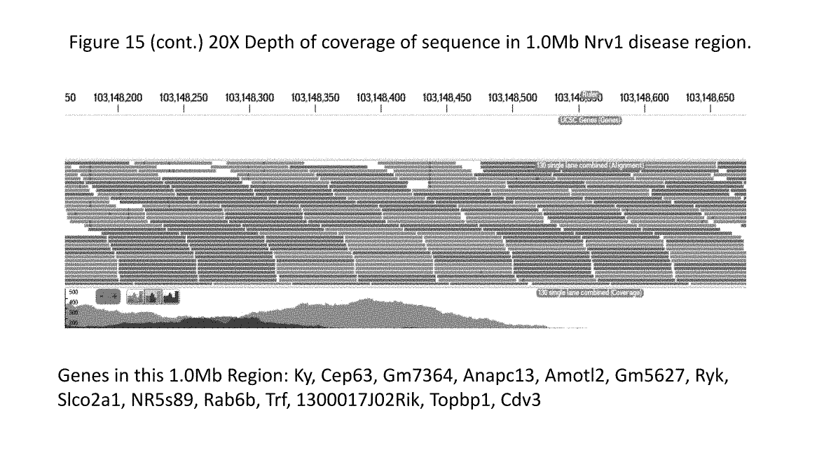

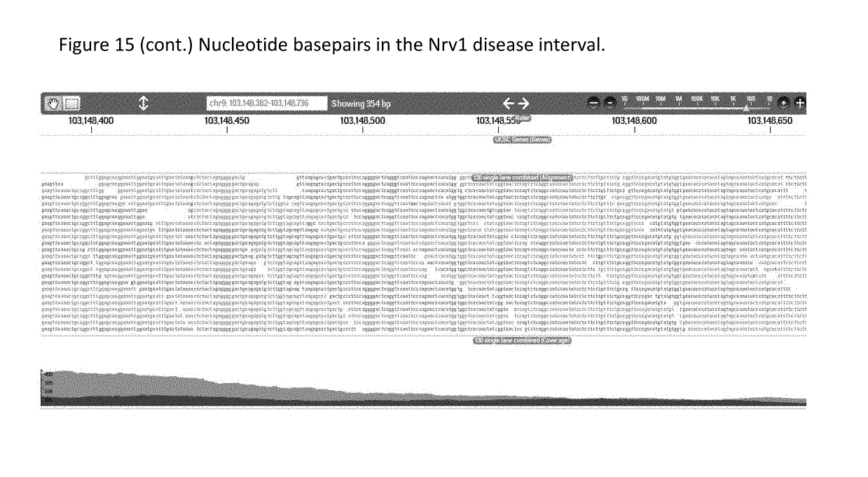

FIG. 12 is a schematic depicting genome-wide scan—the backcrossing strategy. FIG. 13 shows that genome-wide scan maps the retinal defects to Chromosome 9. FIG. 14 is a schematic depicting that genetic finemapping narrows Nrv1 interval to 1.0 Mb. FIG. 15 is next generation sequencing data used to identify the mutated gene into the 1.0 Mb.

The candidate genes found within 1.0 Mb Nrv1 genetic interval are set forth below:

| Gene, full name | Function | Mouse Model |
|---|---|---|
| Ky, Kyphoscoliosis | Neuromuscolar | myopathy involving |
| peptidase | junction | postural muscles |
| | development | |
| Cep63, centrosomal | Cell cycle | ND |
| protein 63 | | |
| Anapc13, anaphase | Cell cycle | ND |
| promoting complex | | |
| subunit 13 | | |
| Amotl2, angiomotin- | Angiogenesis | ND |
| like 2 | | |
| Ryk, receptor-like | Skeletal | Craniofacial |
| tyrosine kinase | morphogenesis | abnormalities, |
| | Cell migration | shortened limbs, |
| | Axon guidance | palatoschysis |
| Slco2a1, solute carrier | Prostaglandin | prenatal or early |
| organic anion transporter | transport | postnatal |
| family, member 2a1 | | lethality. |
| Rab6b, member RAS | Intracellular | growth retardation, |
| oncogene family | protein | behavioral and immu- |
| | transport | nological abnormalities. |
| Srprb, signal recognition | Signal | ND |
| particle receptor, | transduction | |
| B subunit | | |
| Trf, transferrin | Iron homeostasis | hypochromic, micro- |
| | | cytic anemia |
| Topbp1, topoisomerase | DNA repair | embryonic growth |
| (DNA) II binding | | arrest |
| protein 1 | | |
| Cdv3, carnitine | TTP36-Abl/PDGFR | ND |
| deficiency-associated | pathway | |
| gene expressed | | |
| in ventricle 3 | | |

The chromosomal region identified in mouse chromosome 9 is syntenic to human chromosome 3. No disease is associated with this region except for Slco2a1 (Primary hypertrophic osteoatropathy) and Trf (atransferrinemia).

Genes responsible for retinal neovascularization are found on chromosome 9, positions 102,450,352 to 103,338,569, as stated on mouse genome GRCm38.p1 (nucleotide sequence provided in GenBank Accession No. NC_000075.6 (GI:372099101), incorporated herein by reference).

Ky, or Kyphoscoliosis peptidase, is a cytoskeleton-associated protease required for normal muscle growth. Ky is involved in function, maturation and stabilization of the neuromuscular junction. The cDNA sequence of Ky in Mus musculus is provided by GenBank Accession No. BC156644.1 (GI:162317891), incorporated herein by reference. The amino acid sequence of Ky in Mus musculus is provided by GenBank Accession No. AAI56645.1 (GI:162317892), incorporated herein by reference. The cDNA sequence of Ky in homo sapiens is provided by GenBank Accession No. BC024014.1 (GI: 21955431), incorporated herein by reference. The amino acid sequence of Ky in Homo sapiens is provided by GenBank Accession No. NP_848649.3 (GI:148806932), incorporated herein by reference.

An exemplary sequence of Ky in Mus musculus strain C57BL/6J chromosome 9, GRCm38.p1 C57BL/6J is provided directly below.

NC_000075.6
(GI: 372099101: 102506138-102546239):

(SEQ ID NO: 1)

GCACCATGGAGCTGAAGAAGGACAGCAACGCTGTGGCCATCGACATGCTG
CTCATCGTGCACTCCGAGAAGCGGCGCGCGGCGCAAGCCACGCACTTGGA
CCCGCAGGCGAACCCGGGCGCGCTGCTGCAAAACCGAGGAGGTAACACCC
GCCTGGCCCGGGTCCTCAGAATCCCTGGGCACCTCGGCCACTAAGCCCCC
GGGTCCTCTCGCCGGGGGGCGCCTAAGCAGCACCCCCCTCCCCAACGAGG
CTAATCCGAACCTCTCCGAACCTCTCCCAGACCGCTGCTTGTGCCCTGGC
GCCAGGGCACCTGCGTTCCTTGGCAACGCACAGCGCACTGGTTGCCCCAG
GCCTGTTTTCTCCCGCTATTCTCTGAAGGGTGCAAAACACATAGAAAAAT
GCTCTTCAGAAAGAAACCAGGGCCTTAGGCTGATCACGTGTAGCAGAGGC
CTAGTCCCCAGTCAGCATGCAACTCACCATAAGTTACCAGGGACCCCAAA
GGGTCCCTGTACTGAGTCAGACTTTAAGATAACTTAGAGTCTCAGTTTCC
CCTTTCGTAAGCCTCTCTTTTCGCCTCCAACAGATATAAGGACTGAATGG
GTGAAGGGAACCTGGGCTGGGGTGGAGTAGTGTTTGAAGTCTCCTGACCG
TACTTGAATTTAGCTTCAATTTTACTTCTTCTGCAGACTTTGGGAAAACG
AAAACTGAGAAGTGATTGCTTATAGGGTGATTGCAGAAGGCAGAATCTGC
CTCTATTCTACACACACACACACACACACACACACACACACAACTTGTAT
AGCGTATAAAGGGTGCCGAACGGACGCTCAGGAAATCACCGAAAAATGTA
GGGGCCTCGTGGCGCTGGTCTTGGAGGGAGCCTCTGAAGCTGACTTGAGG
GTCTAACTCTGCACCACTTCTTTAGCAGAGATGCCTGCCGGCTGCACAGA
GAAAGATGACTGCTACCATCCCACTCTCCCTCCAGCTCCCACGTTTCTTG
GGACCCCCTCCCGCTGCTCTATCTGCCCCCCACTCCCAGCTTCTGTGCGC
ATGATGCACCCACAGCCTGTCCACCTGCTGTGCGAGGGCAGCGGTTCTCT
AGACAACCTTGATCCATCAGCCACATGCGCTGGAGCTATGATGCAGCCCC
TGGGGAGCTGGAAGGGAGGCATTAAGGGAAAGAGAGACTGGAGAACTGAG
AGTGTTCGCAGCAGTTAGAGTTTAGGACGGAGTGTGAGGACGGGAGCTGT
TTAGGTTAATAGATCCTGTAGTTCTCCGATGCTGCTCCTGGGAGAAAGGA
TTAGACCCTCTCAGGGTCTTTGAAGGAGAAAGAAAATGTCCTCCACGTCT
TGATGGGAATGAGATGGCGCCCGTCGCATGTAACACAATCCAGAAGACCG
CTCCCCGAGTGTAGTGAGGCCTAGTTCCAAGTCAGCCCAATAGTCCCCAC
CGCCCAATCAGAGTCCCTTTGCTTCTGACACCAGAGAGGATAAAGAGAGA
AGCCATAAAGTGCTGGCTATCAAAACTGCCCTCAAGGTACCCAAAGCCTA
CCCAAAATGTCCATGACTCCAGAAGAGCATACAAACTTTGACCCTGCTTT
ACTTGGGGGGTAAAGCACCCCTACTTGAAAGGCACTGGTATTTTTCTAGA
AACATTTTCCCAGAGAAGGTTTCTTGAAGATGCTGGGCAGTAGAGAGAAC
CGTGTCCCCTATTCTTTACCAAAGTGAAAAAGAACAGTGTAGGGTCTCCA
AGGTCTGCCATCCATGGAACCCCCAGGCTGTACCGACCTGTGTAGCCACA
CTTTAGGGCATAGCCTGAGGAAGAAAAGCATTAAGTCAAACTACTTGTTC
AGACGTCTCACACAGATGCACAACAGCCCAAAATCGCCCTGCCGACTTTC
AAGTCTCTTTCTGTCACCTGCAGGAGAGTTCTCCCCCTAAGGAACGGCCA
AGGAAGGCCTTAACGCGCCCCCTCCCCACACCCATCAGAGAAGAATAAAG
TTCAACTATTGTCACGAGGCAGACACGGTGGCCTTGTGCCACAGAGCAAG
GAGGGAAGGAGTCAGAGGGACAGGACAAGAAGAGTCACCAGCAATTTAGG
CTGCAGGGCTGGCCTGGTCTCTCTTCTTTTCTCTAAGAGTCCAGCACGTT
CCCAGCTATTTCCTTAGAACAGCGCTCCCCACAACACAGACTATGAAGAG
CAACAGACTCTGCAGAAAAGATCGGGGAGGGGGGGCTGAGGAAGATTTGG
GGGCTTCTGGGGTGAAGGGTTAGAGAGCCAGACCTGAGACTGAGCTTCCT
GGGAACACTGCTATCAAGTGTGCCACAGAGCTGGTCTTGTGGTGGCGCCT
CTGCTCCCACTGCTCCCACTGCCTGAGCATACATCAGGTTGCATGCCAGG
GACTCAATCTCTCAACTATCACTTCCAGCCACTGCCAGGGAGAGAGCTTC
CCTTCTCTTCTGCATTACATCATTGCCAGAGCGAAACATGGGTGCATCTG
ATGGGTCATGCTGAGGCCTACACAGGTCCATACTCTAGCTGCAAAGGATG
CTGGGAAAGTAGGCAACTGTGGCTCTCCACTCCCATAGGAACAGGCAATC
TCTGTATGCTTTGTCCCTGCATACAGAAATAGGCAGGCAAAGCAGAAAGA
AAACTCTCAGAAAATAATACTTTCATACTTACATATTGGAGATGTGGAAA
TGGGTATTGGGCATCTGTATAGCAAAAATTCCTTCTGGAAAATCCTGATA
GCCAATCCCACCATCACTAAAAACTACATTTTTTTTTAAAATTTAAGGAT
TGCATTGTTTCCTCAAATGTGTCTCATTCAAGGGCAGCCTATCTCAGATC
CTGAAAAAACTGGTTCCCACAGCCTTGTTTAGCACAGATGGGTGTCTAAG
TGGAACAGTAGTGCATGGTACTCGCCCTAGGGCTCCCTCCACAAAAAGTC
TCACACCCAATCTTCTCTCACAGGATTCCAAGGTGTCAGAAACGGAATCC
GGAAGTGGCAAGAGTTGGAAGAAAACAGCTTTCAGGGTAAATCTCTTTTT
CTTTCTATTTTCTGTTCCGTCTCTGTGATTCAAAAACCCCGAAAAGTGGC
TGGAAGAGTTGGGTTGTCCTCATTGGGAAATTGGCCCTAACATCTAATCG
GTGAGCAGACATGGCTGCCTGTGGCTCGGCCAAGCCTCTAGGCCTCTGTG
AGCCTGGAAAACTTTCCCTGGCATCGGGGCTCCTGTGTTTCTGGAAGCTG
AAAGTGGGGAATGATGCTGTGAACTTCAGCACCCATTTGTTTGCTGGCTG
AGCTATATACGAGGATTTGGCTTTACTTGGGGTATCGAGGGTTGTGTGTG
TTCCCACCAGAGGCAGGGCTGCTGACATCACAAAGACTATATACAGCTCT
AAAATGTGTTTGAGTTTTTTGTTGTTGTTGTTGTGTTTTGTTTTTCTGGT
CAGACTGGGATAGTGGCCTGTCTTAAATCTATATTACTTGAGGACTGGGC
AGAGCCGATCAAAGGTTGTATAGTAGTCCCTACTGCCCTGAGCTTCCGAA
TACAGCTAGAAGTTCATTTCCAAGTTCATTTCCAAAACCTCTTCTTTCTG
GTTCCAGACAGCGCTCTTTTCTGGAACACAGGCGATGGGCCAAAATAGCG
GGTTTTGTTTTGTGTGCCTCAAAGACTGATTACCTCAGCTTCCAGGGGGA
TGCATGAGCGTTAACATGCATGATGAGAACACAGACTTCTACTAGGACCC
CGACCTCCACTCGCAGAATGTTGCTGAGGAGTTCCAGCAGACCCCACCCA
TGTAACCCTGGAGTCAGATGTCTGCCCCTGCACAAGAGCACAGCTGAGGA
AAGGTGAATAACTCCGCCTTCGCCAGCCTTCAGACAGCGGCAAAACGGAA
ACGCAAACCAGGAAAACTGATCTTACCTCTGTGCTTATTCCTTCAACAGC
TCCGTTTCGCAATTCCTTTGCGCCAGGCCAAAGTTTGTTAGAGATTGAAC
AGTTAAACAAGCAAGTCTAGAACCCTGTCCTGGTAGAGCTGTGTTCAAAT
GATGGTCATTTCCCCACGTCCCTTATGCTGTTCACGGGAAGGAGTCATCT
TCAAGATGCAAGCACACCCATTAAGAACATCCGCCTGTGTCCTGGCTAGT
GGATCCGTGTAAAGGCTTCCTTAATTGGGGCTAAGCTATAAGGAAAATCT
AACTCACTTGGGGTGGCAAAGATTGACATTTTAGCTAGTGAAGCGATACT
CTAGGCCAATAAGAAGCCTAAGAATGAGAGTCCAGGAACTCCAGCCCTAG
GAATGGCCACTCAGAGCCTGTGTGCTGTGTGCTGGGTCCATGTGTGAGCA
AGAAAGTGAGTCAAGTGGGAACACTGGACAAAAAGAGAATATCCTTTATG
TTGCTTTTCTCTCTTGCCTGTGTGAGCCATCTGTCAAGCACCAAAGGCCT
CTCAGAAAACCCTGGAGTCATTCCACTTTGGGCATCTTCAAGGTTGGAAT
GGAGATTAGTGAAAAACTTCACAAAACAGAAAGCAATGCAGTAACTCTCA
AGATGATTATTTAAAAAAAAAAAAAAAAAAACAACCCTCTGAGGCTCTTA
GTATCCATCCACAGAAGGTGATTTAAATAGTCCATGAAAAAAATCTAGCG
TTTGAAGATTTGGTATGACTCTGGCAGCAGAGAATGTCTGTAGAAACCTT
AAATCCAGGAAACATGATGGTTTCGCCATCCCACCAACATGTGTAATTTT
AATGTAAGATTTGGTCTAGAAACCAGGCAGTGGTGGCACATGTCTTTAAT
CCCAGCACTTGAGAGGCAGAGGAGGACGGATTTCTGAGTTCAAGACCAGC
CTGGTCCACAGAATGAGTTCCAGGACAGCCAAGGCTACACAGAGAAACAT
TGGCTCCAAAAAAAAAAAAAAAAAAAAAAAAAAAAAAAAAAAAAAAACCA
CACAAACAAACAAACAAAAGACTCAGTCTACTTTTTAAATGACATGCACA
TATGGAGAAATATAGCAGCCTCTTTCCACTTTTTTTGAGGGCAAGCTCCA
GGGCACTCGCGTTGAAGCTGATTTCATCACATATGTGCCTTGCTGGTACC
CAAGTCACCGAGTCCTGCTAACCAAAAGGCTGTCCAGACCCGGTAGTTGG
AGAAATTTCATCTCAGAAGGAAGTGGCCCAGTGGCATGTGCTGACAGAGT
GTGCACAAATGGAACCAAGTGTGGTGTTGATCCAAAAGGGCACATTTGTC
CTCCTATTGCTACTCTGTACCCTCCTTGGGCTTGGGTGTTGGCGAGAGGT
AGGGTGGGGAGCCTGAAACACTCCAAACTTTTGGAAGCAGCAGGTGTTTG
GGCTCAGCCACCCCTCTCCCAAAGCTCGCCCAGCAACGGGCTTACCCTGC
TGTGATTGGCTGCTCTCCTTGGTCCTGGCTGAAGTCTGCCTAGCAGCACA
GTGTCTGGTGCCCTACCGCTAGAGAGAAACCACTTGTGCAGCCCTGACTA
TAAATACCTCCCAGGAGCTGTGCCAGCCAATCATCCTTCTTAAGGATTCC
CTTCCCGGCTCCTGTTTCCTCATGGAGGTACAGCCAGCCGTGCCCACAGA
AGGCAGCCTCCCTGTCCTCACCCGAGTGGCCAGGAGCTCCAGCCCAGCAT
GCCACATCACCCCACATTGGCATACGATGACAGTGCATCTGGCCTTGGCT
AGGCAACCATGTTCTAGAAGTCCTGGGGGCCTGACTCAACCCGATCCTAG
GAGAAGACTAGAGCATGCCGGTCCCCCAAGGCAAACCTCTGCTGGGTGGA
CCCCAAGTCTAGCCCAGCTGAGTGCCCCACTTATCCACCTTCACCAGCTT
CCGCTAGGGAAGCATCGAAGCTGTGTCATGGGAAGCACAGAGTTGCCTTC
GTGATTGGCTCCACTCTACAGACAGCAACCTTAGGGAAATGCTAGTTCCC
AGGCTGAACCACTGTTTTCCCCTAAAGTGAGGCTCACTCTAGGTCACAGG
GAGATGACGAATTATAAGGGGAAATATAGACTGAGAACTTCCTTCTGCCA
TAATCCTGGTCCTTCCTCGACTCTTCCCACATCCATGAAGACAAGGTCCC
AGGGCTGGCTCAGTAAGGACAGTGCTGGCACAGCCACAGAGCAGCCATAA
AGCAGAAAGGAATTCATGCCTAGGAAGACCCGAGAAGACAGCGATGGCCT
GTAGCAAGAACCAGGCATCTCTCTTCAGCATCCCAACCCCACTGGGCCAT
AAGGACATGTAAATATCACCAGTGGACACAGAGAACTCAAACATGTTGAG
TGCTCTGTAAAAATGGTGAAACTAAACCAAACTGAGTATCCTCCCACCTT
CAAGGATAGAAGCAGAGGCAGGTGACTGTCCCTGTGCTCCTCAGAGGGAG
GAAAGAAAGCTGAGCTAGGAGTTTGGAAGCTCAAGAGTTCAAGTACTATG
TTGGACCCCCACCCTCACCCCCACCCCCACCCCCGCACAGTTCACTCCAC
AGAAAGGGATGATGACAATCGGTTAGGCATCTGTGTCTGAGAAAATATTT
TGGAAATTGTAGCAAAAACTCCAGAGCAGCAGTTCCATAGACCAGTAGTA
TGAATCCCGGCCTGGGTAAGCTTCCACTGGGAGGCAGAAGGGGTGGGCAC
TTGTGGACTCCCATGGGTGAGGCTATGCTGGTCCAGAGGTGTGCTCACTA
TGGCAGTAGATGTGCTCAGCCCCCGCCAACATACAATTATCTCAGGTGCA
CCCGGCTCCCTCAGCCCCCCTCCCAGGGAACATTATACCATGGTCATGGT
GGTCACTTGCCATCGTCTTTGTCTTTCTTAAATAGGAAACTTGCCAGAGA
AGCAGTGCCTTCAGCAGCCGCAGGTCATCACCTCCTACGACAACCAAGGT
AAGAGACAAATCGCTAATGGCCATCTCTCCTGGGGTTGTGTTCCAGGCTC
TGCTGCCCAAGTCAGCGAGCCCGGGATACAGAGATCAGGCTGCAGGGAGC
ACCTCTGCTCTGATCCATAAAAGCTATAGGGAGCACCTTCTCTGTGATCC
AAGAAGGCTACAGGGAATACCTCCTCTGTGATCCATGAAGGTTAGAGCAT
CCTTCTCCCTGCATCCCAGAGGAAGAACTGAGACACCCTGCCCTTCCAGT
GAAGAAATAAAGTCCTTGGATTACAGAGTCCCACCATTATCGATATTGTC
AGTGCTACAGCTAACACTCACTGAGTGCAAAGCATGTGTCTGCGGCTCTA
CCAACCTCTATACGCACTTAAGTATCAAAGCAACCTTGACTGGGTGAGAT
GAGGAGACTAAGGCTTGAGACAGCAAGAGTCAGGGCTAGCTCTCTGGTAC
CTTACCTCCAACCCCAGCAGGGCCTCAGTTGTTAACCTTCCCGTGGCTGG
GCTTCTGCGGCTTCCTCGAAACTGATCTCCTGCTGGCTCCCAGTATCCAA
GTGCTCCCACATCCTGAGTCACAAAGGCTCCTTGACCTCTCTTGAAGCTG
AACCTGATGAAACATCCCACTTAGCGCCAAGTTTTCGGCAGCAAGGTGGA
ACAACCTGGTCCTTCATTGAGAGCTCTGATTTTCCCAGAAGTGCTCTGGG
TACTTGGAAATGGCAGGCAAGGGCACTCTCTGCTGGCCAGGCTGATTCTG
CAGTTTTCTAGATCTGAGACTACCACGAGGTAGAGCTGAGGTTTCCACTG
TGGCTGGAGGAGAACCTATTTAAAGGACTCCAGCATCTACCATGACTCTT
CGTCAGAAGCCTGGGGTGCTCAGGCTCAACCCAGAGTTGGCTGCCATTGC
TGATGCCATAACAAGCAAGAAAACTATGCCATAGCACTCAAGGGACATGG
GTATGCCCCTTCCTGAAGCACAAGAATCAATGCAGGCCAGGCTGTTTCTC
TGACACTGACGTGGCTGTACAAGTCTCACAGCAGAGATACCTCGCTGATG
TAGAAAGATTGCGAATGGGCTGCCATTCTCTCAACACAGGGGACAAAAGA
AAGGCTCCTGCATGTAGGACTGGGTCCTAGGGAGCGGGAAAGACCGAAGG
GGAAATTAAGGCAGGAGGGATGGTGGGGGCAGGGGTGATAAATCAGTTTC
CTGTTGCTCATTGGCTAGAAACAGCAAGATTTCGCCTTAAGCTTCTGGGA
CTGAGAAGGCCGAAGTCAGCTTTGCTGGGTAAAGGGCAGGTGTCAGCGGG
CCGCAGCCTTTGAAGTCTTGGGAGGGAATCCATTCCTTACCTCCTCCTGC
TCCTACAGGCTGCCAGCATTCCTTGGCTTTATGGCTGTGTCATTCCAGTG
ACGGCTTCTATTATCACATGGCCCTCTCCCTTTTTAGGTAGGCAGTTCTC
CGTCTCCCTCTTATAAAGTTATACGTGGTCACATCTAAGGCCCAACCAGA
TAGCCCAGAATAATCACTCTGAGATCCATAGGTAAGGCCCCATCTGCGAA
ATCTCCCTCGCAGTCAGAGATTCTGATGGATACCCTGAGAATTCATTACT
CAGCTCACGATAAGGAACCATTGTAAGCAGAATAGAGGAGGCTGGGAGTG
TAGCTTAGTTGTTTGGCTATCATGCATGAGGTTTTTTAGTCTGATCCCTG
GCACCCCCCACCCCCAGATAAAAAGAACAGGTGAGCAGCAACTGAGATGG
AATATCTGTGTAAGAAAAGAGGGGGTGGGACAGACACGAGGGCATCTGGG
GAGAAATTTTAGCTGGGAGTCATGACATGAAGGCACAGGCAGGCAGGCAG
GCATCTGCTAGGTGTGGAAAGTCTGTGTCTGTGGCCAGGGGAAAAGGGGA
CATGAGCAGAGTAAGAAGCAGGAGATGAGGCAGAAAGGATTCGAGGCAGG
ACTTATGGTCCTAGGTGTGGGCTGCCTTCAGTGTGCTGGCTTCTGATCAG
AGTATGATCAGCAGACATCAGATGGCTTTAGATGTAGTGCCTCTAATATT
GGAAGAATGAACAGAACAGAAGGGGGTGACTAGAAGCAAAGAGGACTCCC
CCTAGAGGTTGTATCCAATGGCAAGAGGGTTTCCCATGTTGTGGATCTGG
GCTAGAAGGAGAAGGGGGAACGGGGAGACAAGAATTGAAGCATAAGCCAG
GGGTAAGACACAGCAGACTGATCAGGGGGCGGGGGTGGGGTGAAGGGGAA
GAGTTGAATGTCGTTCTGTTGAGACTAGGCATTGGGGAAACAGAAAGGAT
GTCCCCATCGTGTGTTGAGTTTCTACAAATCGGCAAGCCTGGGACTCCTT
GGGCTCTGTTCTAGGGACCCTGTCAGCTCCAGCACAGGAGGTCCAATCAT
AGCCTAGATCAGAGGCAGAACACGAGGTGTGTGCCAGACATCGAGCTAAG
CACTGTTCATGGCTCCTCATCTCCCTCAGCCACACTCTATGAAAGGTTTT
CTTTCTCACACACACACTAAAAAAATGAAATCCACTTTATGGATGGAAAA
AAATGGAGAAACCGAGGCACAGGGGCATCTAAGCAGCTTACCTGAAGTTA
TGCACTAAACAGGACAAGTCAGATCCAAGAGTTGAACATGTCTGTGCACC
CCCTTAAAGCCAAGTGGGCTTAACTTCTGTACCACACTTCCCCCATACAG
GACAGGGACCACAGAGCTGATTTGAGTACAACGTCCCTGCCCACCCCAGC
AAAAAGTCCAAGGCTCCAGATGGAGTTTCTTATTAAAAATGGCTCTGCTT
TCCCCTTCTCCTCAGTGGCAGGTTTTATACAGCAGGCAATAAAAAGCCTG
CTCGTAGTGCACAGGGGCCTTAGAAGATGAATCTTCAGTGGGCTTGTGTT
TGGGTGACAGGAGAGTAGTCACCACAAGGAGGCACGATGGCTTCTGACCC
TCAGGGTGGCGGCTAAGGAGACAGTTCTGAAATGACACCACCCATCATGC
GTGTCCCCAGCTTTTGGAGCTGACTGCTTCCCGACTACTACATCCTGGAA
GCATAGAGCACGCATCTTTTAAGATCTAGCCAGCCATGGTTCATTTGAGA
GTGCTTTTAAGATGACGAATGTTTAACTAAGATTTTTCCGACTTTAAGTG
TTATGGAAAAGTATGCCCAGGTTGTAAATGGATCAGCATGTACAGAGACT
CTTAAGTCAATGGTGCTGAGTATCTTGCATAAATCCTGGGACTCCTCCGC
ATGCACCCCTGTCACCACCCCCATGCATGCATACACACCATGCACACATG
CTTGCCTGGCGGTCTTTGTTAATTGATCAATGCACCTGGCAGCCACTTCC
AATCAGTCAGAGTTAATGAACAACACAAAACCCACTTGCCGTGCCCACGT
TAGATATTCTGAGTCAATTGAGCCGTTAGACAAATACCGACTGAAACCTG
GAGCCATTAGGAGAGCTGTCGTGTGGGAGATGTCACCTGACTGGGTAAAA
GGCCAGCCTAGAACAGTGCCCACACAGACTCGGGATGCTCTATGCATCTC
AAATTTTGAGAGAGAAAGGGAGAAAAGTCAGCAGGGTTTAGAAAATGTCC
AGCCTGAAAGCCACCTCATAGGGGTAGCAAGAAATATCCCTAAATGGCCA
AGGCTGCAGTTTCAGAAGTCTTATTCAGCAAATGAATTGCTGGGGAAGGG
TACTATGAGAGAGGCCAGGCAGGACACCCAAAGTCCCAGAAAGGGTCTCT
TATCCTTGACGGTCAAGGTTGGTGTGATTGGCAGCTGCTTGGGATTTAGC
TCTGACTTGAATTTTGCCTGAGATTCTTCGTGTTGAGAGATAGGTCAGGT
TAATATTTTTACTAAAATCTCAGTTTTATTAGATCTAAAAGTGAGGTGGG
TACGCTGTCTGCCTCTCTAGTGAATGCTAACCATCTTGACACAGGGTGAT
GACGGCAGGCCATTCCTAGGGGGCAGAGAAGTCTCACAGATGGTCTCTTC
AGTCTCTGGAGGAATGCAAAACTCCATAATCAGGGTGAGAGTTTGACAGC
GTTGAGAATAAAAACAGCAGCACAGCCTAGGGACAGTTGGTCCTGGGCAA
GGAGATAAAGTGAAAAATTGCAGGGAGAAGAGGGGGTTCAAATGATGTCA
GGGAGAGCTGGTGAGCGACAGCCCCTCTAAACATGAGGCCACCTCCCCGT
TAACCCCAGAAGGGTCACAACTGTGGTTTGTTTCCCAGCCCCACCACTCT
TCACAAGAAACATGGTTGGAGGAGGGCAGCTAGATGTACATAAGCTAGGA
GAAGTGCGAGGGAGATGCTTACTGTTGGACCCTAGGCCCACGAGCTGTTC
ACCTTCGCAGCTCCTTTAAGGCTACAGTTGGTTCAGCCCAGTAATCTCCA
AACAACAGCTGCACTGGGTCAGACAGACTCCTGGGTCGGATAGACCCCTT
TGCTGCGGGACTGAAAGCATTGCGGGTTGCAACCCACAATAGAGAGAGCG
TGCAGGGGAGCCTCTCACAGTTCATCTTTCAAAAAGCATCTTGTGGCCAC
ACACCACTTTGGAGAACACCTCAGAAAGAGTTGCACAAGAAAACAAAGAG
GCCAAGTTAACCAGGGCCGCAAAGGGAATATGCTTCGGATGAGGGTGGCA
CCCAATCTTCTGACAAGAATGGACTCACACCCTGAATTCTGAAATCCTCG
GCATCTTCATCCCAGGATCACAGGGCTGGTGTCATGGGCAGTGAATGTGC
TAGATTCTACACTTTACAGGTGAATGCTGGGCTACAGAGATAGCTCAGTT
TTAAGAGCATTTGCTCCACAATCGTGAAGACTAGAGTTCGAACCTCAGCA
CCCACATGACAGGGCAGGCATCCTCTGACATGCCTTTAACCCAGCTCAGA
GGCTGTCAGAGATTGGAGGCTCTTGGACTTGCTGGTTTCCAGCCTAGCTG
AAACACAAGCCTCGGGTTCAGGGAGAGACTCTGCCTCAAAGAAATAGGCG
GAGAGTGATAGAGATCACCTTATTTATTGCATGTGAACAATACATATATA
CCCCCCACACACACACACACACATTAACTAATTTATTAGTTAATTAAATA
TTTAAAATGTTTGCTAAGCCTTTTGGTCTGGGGAACACACATTGTATCTC
CAGTCTCGTTAGCATGCAAAACAAGTTTTAAAAGCGTATAAGAAAATCAA
TAATGGTTTTCCAAAAGAAAATAATTGCAGTATATTTTAACAAAGGATTA
ATAAAAAGGAACTCATTCTTACAAAACAAAGGAAACATAATAAATCAGTA
AATAATAATAACAATCAGCACTCAAAACAATTTAAAGAGCAAATAAGAAA
TTTTAAATCTACACTAAAATGTTTTTAAATACATAAAACAATAAGGGACC
CTTCATTAATAAAAATGTAAAGGGTTTATGAAGGCAGGTGCCAGTAAGCT
TAGCAGGCATGTTTGTGTTATCCACCACCACGGGACTCAGCTCCTAATGC
CATCGTGAGCTTCCAAGCCTGCAGATCTGCTGGACGGTCACTGTCCACGC
CATAGCCGGCTGATCTCAGCTCTTGGCTGAGCTAGTGCTGGCTGTCAGCT
GGTGGGCTGGCTGGAGACCAGAGGGTTTCAGATAGCCTCACCTGAGCAGC
CCTGCGCTGCTCCCTGCGGTCTCTCACCCTTAGTCTGGTGCACACACAGC
ACAGCACTGTGGCAGGGATGCCAAGAGTGGGAGCGAGCGTTGCAAAGTCT
CTCCAGGGCTAAACTCAGACACTGAATGATAACACAAGTTCTACCCCATG
CTAATCAGCCATACTAACTTGTGAGGCCAGCCTACATTCAAGGGGTGAAA
CAAAACACCTCTCGTTGGGAAGAAGATGAAGTGTAGGCAATCAACCAGGG
CAAGAATGAGTAAAAAGGGCATTCTCCACCTCTGACCCCATTGGTGGAAT
CAGAAACCAACAGAAGGATGTGTGGGAAAGTCGGCCAGTATTACAAAAGC
CTTCCAAAACCCTCATTCCTTTCCATCAGTGTTTAGGAATTCACTCTCAG
GAATTGATTGGAAAAGTGTTAGAGATGTCTTCTTTATCCTGATGTTCACC
ACATCATGGTTTATCACAGCAAGCACCTGGGGAGTGCGGAGTATCCGATG
CACACAGCAAGTTGTAGTGAGTACTCTGAACACGTGACGTAAATGTGTGT
TCCACTGCCTTCCAGGACTGGGGGACATTCTCTACAGCACCGCAGAAGCC
ACGGCAGCTGACAGCACTATTTAATGCTTCTCCGCAGTTGCTGATTTTTT
TAAATTAATCCCTTCAATCTCTGAAAGAAGAGAGCTGATATCTCTGACTA
TAATGTATATTTGTCTATTCCTCTCTATAATTCTGTGAGTTTTATTTCAG
ATATTTTAAATCTTTGTGTTCAGGTGACTACGTATTTGACATTGTCACAT
CTCCTTGATAAGCTGACGTTTCTTCACTATTATATTATACTATTATGTTA
TATTTCTCTATCTCTGGTATTTTCCTTTCTTGAGTGGATATGTTAATGAG
CAAATTGTCTTGGTCTCACTGTACCTGAAAAAATGTTTTTATTTCATTCT
CATGTGGAATTTTTATTGGATAGAGTCAGTTAATCCCTTTCAGAATCTTA
AAGATTCCACATCTTTCTGTCCTAGTTTCTGATGAGAAATATGTAGCCAC
CTTATATATAATATATCACTTATCCAGAGCGTATTCAGGGACATTGTATC
TCAGTGTTGTAATTATGTCATGTCTGGACCTGGATCTTCTTTTATTTATC
ATGGTTGGCACACACTAAACTTGAGTCCATTCATGTATGTCTTTTCTCAA
ATTTGAGTAGTTTGTTATTTCTTCAGCTACTTTTCTGCACACATGGTTGC
CGGAGACATGAACAGTGGACTTCTAAATGTGGTCCTGTGGTCTCTGAAGC
ATTGCTGGCTTTTCCCATTGTCCAGATTGGATCCCGGCCGCTGTTCTCAT
GTTCTCAGGCTCTTTCCTTTGTCATAGAAAACATTGTAGCCCATCTTCCA
TTTCAGGGTTAACATCTCCACTTTGTTCTTCCATCTTCTGTTTCTTCGCT
GTGTTTTGTATCTTTTGCTTTATTCTGAAATGGCTGGCCTTTCGTTTTGG
AGCATGGCTAATGTGCCATTTTATCACCTTTGGGTTAAATGTGGGTTGTC
ATGGTTGGCATCTTTTCTTCTCCAACTGAGATCTTCCCAGGTACTCTGCT
GAGCAGTTCTGAGTGGTGGCCTTTGCGGGGCCCATCTGCTTGCGTGCACC
AGGAAGTGGCCAGTTCAGAAGGAAGACATGGTTTTTCCCCTAGAGTTTGG
TAGTCAGACTGTGGTATAAAGTGCATGGGGTGAGACGTGTGGTGACCATG
GGAACTCATTTGCAACTTTAGGGAATTACATTTCGTCAGCCATTTCCTGT
TCCTCTGGCCAAAAAAAAAAGGTTGAAACTTTGTCTTTTCACTCTGTTGG
GTACTTTTTATAACTGTGTCTGGGGACAATCAGCAGGAGAGATGAAGGAG
GGAGGAAGGGAGGATAGAGGGAGGGAGAAGAGAGGAGAGGGAAAGAGAGG
GAAGGAGAGAGATGGTGGAGGGAGGGATGGAGCGAGGGAGGAAGGGAGGG
AGGGTGGAGGGAGGGAGGGAGGGAGGGAAAGGAGACAGGGAGAGAAAAAT
ACACATGCCACTTGTGCCTGGCTACTAACCTCACAGCTACCCACATTCTT
GCTTCTGGGCTGAATCAGGAAAATATAGAATAAAGAAAAAAAAAAAAAAA
CACAGCAGAAGATGTCCCTGAGACAATTAGGGGTTCCCATTCTCACCCTT
CAAGTAGAAAATGATTTTTTTTTTCCTTGAGTACTCTTTTACATCCATGC
ACCTCTGCGTAGGGATGCCTTTGCCTGAGGTGTTGCCAGAATGAGAAGAA
GGGTAGACCGATGCGGTCCTGTCTCACAACCCGGTCGTTGCCACTCCCAA
CCCTGTTCTTTTCTATCACTGAATTTCCAGGGTCTGCAGATGGGTTTCCC
CATGCATTCCACCCCCATGGTTAGATGGGGTAGAGAGAGCGCTTGTGTTT
GTTGTTTGGGTTAGTCTGGAAAACATGAATTATCTGCATATTCAACAGAA
AGATCTAAGATGAAGCACGTTCTGCCATTTGAACATCTGTCACAAGACGG
AGTGCCCTTCCTAGCAGCATTTGGCTAAGAATGTCACCTCCCCCTTTGCA
GGTACTCAGCTGACTGTGGAAATCCACCCCCAAGACGCCATGCCCCAGCT
GCTTAAGAAGTTCTCTTTGGCTAAACGTACGTACCTGTACAGGGCTGCTT
TGATCGTCCAACCTGGGAGAAGGCAGAAACATTACTATTTACACCCATTG
TGTTAGTTTCTATTCTTATTGCTCCCATGGCTTTGGGAGATACAGTATAC
TGTGACAGAAAAAGTTTGGCAGCAGGAAGTTCCGTAATTGCAGGAGTATG
CAGCTGGACGTATCGCTGCCCCACATAGTGGTAAAACAGGAAGCAGAAAA
TGAACAGGAAGTGGAACTGGGCTTTAGAGACTCAAGACCTGCCCACCAGT
TACCCACTTCCTCCAGAGAGGCTTCACCCCCTAAAGGTTGTACATCCCTC
CAAGACAGCATTAATATCCAGGGACTGGGTGTCCACACACATAAGTTTAT
GGGAGCATTTCACAGTCAAGTACAACAGCCAGATGCTACTGTTCTCGGGT
CTCTGTAAGGAGGTCCGGGTGATGCGCATAACTAATAGTGCATTCTCTCA
CCAGACTGTCACTACCAGGGTTTTAAGATGCCATCTGTAGGCTGCCAAAA
ACCGTGGTATGCACAGTGCTAAGTACTTCCCATGCCTCACCTTCTTGACC
TTTATGCAGATGCTGGTATTCGCCCCATGTTTTTTCAAGTCAGCAAAGCG
AGGCTCAGAAAAGAGAAACGACTGTCCAGGAGAGTTACTCGGCCAAAAGA
TAAAGACCCAGGTCCAACTCCGAAGCCTCCGATTTCAGCTATAATATGTG
GGAGGTGTCAGTCTATGTAGGCAGGCAGGATCTTGACTGCAATGACTCTT
CATCCCTGCCCCATCTTCACCCTGGCCTGCCTCTTGGATGAAGGAGGCCT
CATGAGGAAAGAGGCAGAGGAGGCTTGAGGCCCCTCCCGCTGTGTCCTCT
CAATGATGCAAAACCCTCCAAGGCTATATCCTTGCAGAGCACACCACGAC
CCTTTTTTTTTTCACATAGAAAAGGAGGAAAAATAAGTAAGATACCCTTG
TAGGATTGTTCTAAACTCCCATAAACACTAACTTCTCGGAACCCTGCCAT
TCCGTCTGAAGATGGAGGAGGAGGGGAAGAAAAGTTCCAAAGAAAAACAG
AACCTATTTCTGGGAATGGAAACCACCCTCTCAGCTTTCAGTTCCTATAT
TTACAGCCTGAAGTGGCTCTGGGGGCATAAACCGTCGGCCTCGGTGAGCC
CACCCTCAATTCTGAATGGTTTCATTAGACACCATGTCTTCCTGACCTGA
CTTTGAACCAGCAGCTAAGTCTCCTTTGCTTCTTTCCCAGGGACTCCCCT
GTTACTCACCATTCACAGTGCAGGGCCACCCACTCCAAGGGCTACATGAC
CTTCTGGGCCCCAAGTTCCATACACATTACCTTTTGTGCCCCTATTATCG
CTCTTCGCTTAGTCAGTTCTTGTCTTGGCTAGACATAAAACTGCCTACAT
GTACTGACAACTGCCACGTATTATAGCTAGAACCAAAGGGTTTGGAGCTG
TACTTGGGCCTTATTCTCTTGGCCAGGTAACTTTCTTGGACAATCATTTC
TCCTACCTGAGCCTCACTTTGCTGACTTGGAAAACACAGGGTGAATACAG
GTCAAAAGGCTAAGGCACAGAAAGTACTAGAAGAAACTGGTTTGTACCTA
TAGCGTCTGCAGCTATTTTCCAAGATGGCAGAATGCAGAGGAGGGGTGAA
CCACAGCTCTGTGCAGGCCTGCTGCAGGGGATAAAGCCTTGAGATCACAG
CATGCTCTGACTCAACTCAAAGACCATAGCTCCCTTTCCTGTCATTCATC
CCCTATTCCCAGTCTCTTCTCTGGCCCAGCAGCCACAGTGCATGGTGGGT
GGGGATGGGAGCAGGGAGTCTGTAAGAGCCAGCATGAACAGGGAGAGTGG
CCAGGACTACTACCTAGAAAATCGCCAGTGCTTGCTTTTCTGTTTCTTCT
GTGGTATCTCTGACCTTTGGGATATTCCAGGTTTACAAGGTGATAAAAAT
GGAAACATGAGACCCCGGCAGCCTGGAGGGAAAGATGCCCACGGTGAGTC
CCAGGCCTTCTCTGGGGCCTCTTTAAAAAAAAAAAAAAGCAGTGAGTTGG
CCTCATGGTCCAGTCTCCAGCTGCCAATTGGTACTGGCTTGCTGGGACAT
CAAGAGTCTGAGACCATCTGTGGATCTGGGAGAAAGAGTGTCCTGATTGA
CTAGTAGTGTTGGCCACAGACACAGGTGAAGAGGGAAAACTCTTATATCA
TCCCAGCGCCAAAGAGTAACCCCTTTCTGAGTACAAAACAGTCTACTACA
GAAAATGGTGGACCCGGGATGCAGAGTGATGCTAATTTGGAGCTGGAGAC
GTAGCTCTGCTGGTAGGGTACCTACCTACCTAGCAGGCATGAAGCTCTGG
GACCAGTTCTCAGAACCACATCAACTGGGTATGCCAACATACTCCTGCAA
TCCTGGTACTCAAGAGGTGGAGGCAGGAGGATCAGAATTTCAAAGTCACC
CTGTGCTCTATGGAGAAAACCAGCCTCAACTGGAGACCCTATCTTTAAAA
AGAAAAGAAAAGGAAAAAAAACAGTAAAAACATAGATGAGGGACTTGGAA
TACAGCCCACTGGCAGAACACTTGCCCACCATGTGCAGGGGGTCAATCCC
CATTGTGACAAAAAATACAAAGCAATAAATAAAATGGAGTGTCAAGTGTT
TACACGGTAGGAAGCTTGTGGGTAGCAGGTTACGGCAGAGAAACACTAAA
TGGGGTGATATGAGGTGATAGATGGTCTGGATTGGAATCCTGCTTTTTCC
TCTAATTTGTCTTCTCTAAGCCTCAGTTTCCCCCTTGGTAGGCCGGATGC
TAGCAGTGGCTCCGTGCACACCTGCCTATTTTATTTGGCCTCAAAATCAG
AGTAAGTTAATTAATGTTCCTGGTGTTTTAAGTATCTTGCTATAAAATCA
TATTCTCCTGTTTGACTCTTACATAATCTGCTTGGAAGGTAGATTTAACG
AGGCATGAATCACCATGGGTATTTTGAATATAAAATATCAAATATTGTAA
GAATGGAACCAGGGAAAGAGGAACTAATAAAGTGGAAATTTTAACACCAT
TTTAGAAATGCCATCCTTCTAAACAGGAAGGCGGGGAAGAGCTGACCTGA
GCGGCAGCCTATGTGATGTGAAAGCCCACCTGATGCCCACCTGTCACCGT
GGCGCTCTTATCAGGCAGTGACGGGAGGCAGGCTGAGGAGAACTGAGATG
TGAGGCACTCAGCAGGAGCTAGCCAGTTATTCCCCTTCTGGTGGGGGCGG
CTGAAGTATGGCTGGGTTTCGGACCACACTAGATGGTTATATCTGCATTA
AGATGCCCCAGGTCCAACTCTATGGGTAAACACAAACCAGGATGATGTCA
CCTCCTGAATCCCTGATCTGCCTCCCAGAACAGACTGGGGCACTCCTACA
TCCAATTGGAAAATAGAGGTGGACACTAGCCATAAACCAATATCAGCATC
CTATTCTCTCTCTCCCTTTCTACCCCCATGGCCTGACTCAAAGTCTGGGT
GCCCTACTGAGACCCTGGTGACCAGCTCTACCAGACTTCCTTACACCTAG
CATGCCTTGTGGTACCTTACACCAAACACTTGGCCATGGGTCCTCCCCGA
TTAAGAATACCAGGGGCCAGCAACACTACTTACAGAAAGTTAAAAAAAAA
AAAAAAACCTTAAACTAATTATCAGGGGTGGGAAAGTGGATTGCATCATC
CTTTCAGTAATTGTCTGGGTACAATTGAGTCAGCCAAGGTTTTTAATGCT
GAATCTCCAGGGTCTTCAAAATAATCTCAAGTTCCCTGTGAATCTTCAAG
ATGACCATAGGGGTAGTCACAGTTCCTAGGCAACTTTGCCCAAACAAACG
TTTCTAGGAGGTACAGCTCTTGGAATGAGTGTGTTGGTGTAAGAAAGGTT
AAAGAGCCATGGATCCTCCAAGTGACTATTAGGATTGGGCTCACAGCCCT
CCTCAGGGGTCCCCCACCCCACCCACCCCCCGTACTGACTTGGTTTCCCT
GTGTTAACAGCCTACCCCTGGGACAGATCCAGTTTGAAATCCATGCCCCT
GGACCTCCGGCTCTTTGAAAAACTGGATGCCTCTGCCTCACAGGTAGGAG
AATTGACTCCCTTGGCTAAAGGCCCCAAAGGTGACTTGGAGTTCCCCAAG
TCAGGATAAAAGAGCCTACCTCCAAGGCCTCTCTTGATCATACACTGAGT
AAACCAGATGGATGGTCTATGGCTGTCCTTTCACAGTGTACCTTCGTTAC
TTTGTAATGGCTGTGATCAAATAACTGGCAAGAGGCAAGATGATGGAGGG
AGAGTTTGTTTTGGCTTACAGTTTGAGGAGGCACTGTGTCTCGGGGCAGG
GAGGGTATTGAGCGTGAGGTGACTAGACAAATTGCAACCCTAGGCAGGAA
GAGAACAGACAGGAAATGGAGCAGGCTATAAAACCTCAAGGCCTGGCCTC
CAGTGATCTACTTCTTCCAGCAAGGCTCTGCCTCCTGAAGGCTCCACAAC
CTTCTCAAAGAATGCCACCAGCTGGGGACCAAGTGTTTAGATACATGAGC
CTATGGAGGACCTTACACATTTCAATACCACCAGTGTGGATGCGGAACAG
GAAGCCCAAGGGTTGTGTTAAACCCATAGGCAATGCCTTAGTTACTTTTT
TCTTGCTGTGACGAAACACCATACCTTGGGGAGAATAGGGTTTATTTCAT
CTTATAACTTATAGTCCCTTATCCAAGGACATGGAGGCAGGAACTCAAAG
CAAGAAGCCTAGAGATAGAAACTGATGCCTAGGCCATGAAGAACACTGCT
TACTGGCTTGCTCCTCGTGGCTTGCTTTCCTGTAGCACTCAGGACCACCA
GCCCGGAGATGACAATACCTCCCACGTCATTCATCAGTTAAGACAATAAG
ACAAGCTTGTCCGCAGGCCAATCTGGTGCGGGGTATTTCTCAATCGAGAT
TTCTTCTTCCCAAATGACTCTAGCTTGTGGCAAGTTGACATAAAACTAGC
CAGCGTAGATCAGGTTTAGTAAATCTTAACCATGACCACAGGCTGCTGTA
GCTACCCGAATACCTGCCTGGCTGGTCCCAGATGGGAAGAGGAAAGCTGA
GGTGGCTCTAGCACCTGGGCTTCAACTGGCCAGCACACATGCCCTGTCCA
ACTCCCCTAACGCCACACTATTTATCCCCCACCACCCTCTGCCCTCCTGG
GCCCCGTTCCAGCTTCCTCACTGGAGAAAGAGTAGCTGGCCAGATTTGGT
GGGTGTCACACTCCCATCCTCCCCTGACCCCCATAGAATCTGATTTTTCT
GCTCCCAGGTGACAGTCAAGAGTGGCCTGAATGAGCTGGTGAGTGACTTG
CTCCAGGAAGCCCACAGCGATCTGGAAAGGGTCAGAGCCATCTGGATCTG
GATCTGCCACCATATAGGTATGCCACCATATAGGTATGCCACCATATAGG
TATGCCACTAAGGGGGTAGCACCGGGGACCCTGGATGCCCCTTCCTTCAG
ATCCCTGCTTCTGTTGCCTCATCAGAGTGAGGAGTTAGGTCAGGGGGACG
TGCCTGAAGTAACCAAGGAAGGTAAAAGCCACCCTTCTGAACTGGGCCTT
CACCCAGAAGCTGACCTCCCTGGGTCCAGGATTCCTAGTAAGCCCCGCTC
TACAGAGATGAGAACCCAGGACGGCCCATCACCAAGAGGGTGACCACAGA
CTGAGGAGGACACAGCTGTATGAACACCAGAAGGTCCTAAGACAAGTGCC
AGCTAGGGCAAGTGCTCATATTCCTGTCTCCTGCTGTCCCACCTGTGTCC
CCGGCCGCTGGCTTCCACCTACTCCCAGGGTTCTCCAAGCCATCTTAGAA
ACCTGGGCCTGAGCGGCTGCCCACTGCTCTCTGAGAGACAGTTAAGGGAA
TCTTGCCAGGTCCTGACAAGTCACAGGCTTCCCTTGCTGAAGCCTGCAAG
TGGGGGAGATGGTGGCCAACTGAGTGGCGGGGTGGACCCTTGTTCCTTTG
ACATGAAGAACAGCCAGTGTCCCACCTGGATTCCCCGAGGCTCTTCTTTG
TCCTCCAGGTGCAACTTAGAAAATGTAGTCACCCAAGGTCTGAACCTGAG
GGCGGGTGCCTCCTTCCCTACACCACCACCAGCTTTCTTCCCCCTCTTAG
ACAAGGTTTCATTAGGCAGCCCTAGCTGGCCTGGAACTCACTATGTAGAC
TAGGCTGGTCTTGAATTCACAGAGATCTATCTGCCCACCTCTACTTTCTG
AGTGCTGGGATTAAAGGTGTGCGCTATGAGGACCAGGTGGTCCTCTCCGC
TTTGGCCGCGATGTCCCTTTTGCCACCTACACAAAAGGTGACCACATCTG
GCTAGGTTGGGGTCCTGTTTCTGTTCATGCCACCATTTCCTCCTGAGCAC
AGCTGGGGGGTGAAGGCTATCCTATGGGCTCTGTGGCTTTCCCTTCCTCA
CTGAGCCATCCTCGTGCACCTGGGAGCCTTGCGTGCCCACAGCCGCCTCC
TCTGTCCTCCAGAGTACGACGTTGAGGCTGCCCAGGAGAAGGACCGCCAG
GCCTTCAAACCCACTGACATCCTGAGGACCCAGAAGACCAACTGTGATGG
TTACGCCGGCCTCTTTGAAAGGATGTGCAGGTATGAAAGACAGCCTGTGA
TAAGCAAGAGTCCCCGGGCTGTCACCCGACTGCCTGCTTCTTTCTTCTCT
TTCGTCCCTCTGTGCCTCTGCCCCCATTGCCAGGTCTGGAGCTTGAGTTT
GCAAACTCTGCCGGGGAATCCAAGTGAGTCCTGAAAAGGTCAATTTCTCC
CTTTTTAACACCAGCGTAATTGACTTTGGACCATCTGCCGGGCTTCGCCA
TGCATTAACATAATGGCAGATGAATGCACAAAGATAATTTGGTTATAAAA
GGCCCCTTTTCTGCCTTCTCCCTTCACTGTGCAGCTGAAGGTTAAAAGAA
TGGCAGGCCTGGAAGCCAGAGGACAGTGGCCGCTGACGCCAAGGAGACTA
TGAAAGGCTGTCCTTTGAGGAGGGAGAGGGGCCGTGCTTGGAAAGATCCA
TTGGTAGGAAAGCTCAGTAGCCAGGAAGTGGTTCTCTGTGCCTTGGGGCC
CCATTCTCAGAGCTATGGCACAGACACAGGCCCAAGTGGGAGAACTGGCT
GAAGTAATTCTTGGTAACATCAAGACAGTCTCAAGTCCGGGGGAGCCATA
CGGATGGATTTCTGAGGTCACCTGGGTTCTCCTAGGGAACAAGCAGGAAG
TGAGTGATTGGCTATGTCCCTGAAAAGCTGTGAGTAGGTCTCAGATACCC
AGTGTCCACCTATCGGGACTGCAGGTGGCAGTTGTCAAACCACAGTAACA
GATGCTCTCCAGTCGAGCAGCTGGCATGGCTTAGCGTTGTGCTGCTGGTC
CCCTGTGGCCTTGTGTGCACTTGTGTGTGCCCTGTCTTGAGATGCACACA
ACCCATTCTCGGCCTGAGCTCCAGTGCCTCCCAACCCTGTATTACCGCTT
CCAGGTATAGGCAGATACCTGGTTCTGGGTCCCTCTAGCCCTCCTGCCCA
ACTCCCTGCTCCAAACTTCCACTTCCCATCTCTGGGACCCTAGCCTCAGA
GACAACCTGGGCAGGATCCATGCTGGCTGACCCACTGTTCTCCCTCCAGC
AGTACCTTCTACTTCTCCAAGTTCCCAGTGACATCCCCAGCCCCAGAGCC
AGGTGCCCCATTGTCTTTGGGCATCCCTGTCCCTGGGTTCCCAGTGTCTT
TCAAAGTCCTCCAAAACTTGACCTCAGCCACCTGCTGGCTCTGCACCTCG
AAGCAACACTCACACAACACTTCCGCTTCCGCTTCCCATGAGTGCACAGC
CCTTCCTGGCATTTCTCTAGGCCCCACCCCAGAGCCTCCTTCCTATCCTT
TGACATCTGACCTTGTCTACTAACTTGTGTGAGTTACATTAGGTTTTCAT
CTTGTAGCTCTTTACTCCAGGTGATGATGAGGAATCAAATCCAGGGCCTT
TCCCAGGGAAAACAAACAATCTACCACTAAGCCTTAGCCCTCCCATGGTG
CTCCAGACAGACTCTAGCTCTGTAGCTCAGGCTCGCTTTGAATTCACTGT
GTGGCAAGGCTGGCCTAAGTGCTGGGATTATGGGTATGCAACAACTGTTC
TGCTAATAATTTTTTTAACTATATTATGTTTAGTTAGTTAGTTAGTTAGT
TATGCCTCTGTCTCTCTGGGGGCGGGGGAGAGGGGAGGCATATACATGTC
ATAGCATTCATGTGAAGGTCAGAGGTCAACTTTCAAAAGTCAACTCTGTC
TTTGTAACATGAGGGAATTGAACCAGGCTTGGTGGCAAGTGCTTTTACCC
ACTAAGCCATCTTGTCAGCCCACTAATAACTTTTTTGAATTAAAAAAAAA
AAAATGCTTTCTGTAAATAGTCTCCAAAATGTACAAGACAAGGAAAACAA
CTAATTGGCTCTGAATCACCTAGGTAGAGTTGAGATAGTGACGCCCACGT
GCCTTCCCAGGTGATAGTGACACCCATGTCCCTTCCCAGGTGACAGTGAC
ACCCATGTGCCTTCTCTGGCTTGTTCTCAGCACAGTTCTCTCTATGTGCC
TTTCTTCCTGTGTCTTAATCCCCCGCCCCTCTTCTGCCTCTCTGAGAACA
TCTGGCCCAGAATTCTCCACACAGTAGACCCTCAATTGGCATTTACACAC
AGTAGGCTTTTAATAGGCATTTTCAGAATGGTGAGAAGCAAGAAGGATAT
GCCTGGCAGACGTGTGTGTTTTCTTTTGAATAACAGAACACCACCGGGTT
TTCAGTGCCTGCCTGGCATCTTTCAGAATGTGGTTGTTTTCATAGGAATT
TGTAAATTTGAAAACACTTGGATTTCTTTTTCTTTTTTTCTCATGATGGG
TTTTGTTTTGAGGTTTATTTTTTTAAGAAGTATTTATTTTATTTTATATA
GAGGAGTATACTGTAGCTGCCTTCAGAAGAGGGTATCAGATCCTATTACA
GATGGTTGTGAGCTACCATGTGGTTGCTGGGATTTGAACTCAGGACTTTT
GGAAGACGAGTCAGTGCTCTTAACCACTGAGGCATCTCTCCAGACCCTGG
ATTTTCTTTCTAGTTGATTCATTCTTTCACAATTTCATATGCATATTATA
CTTTGAATGTCTCCCCCATAGTCCCCTCACCTCCTTCCCCTCCCACTAAA
CCCCTTCTTCCTAACAGACTCCCCCACTCCTATTTTCATGACTTTGGGGG
CCACTCTATTTTATTAGGGTTGTCTACAAGAGTAGGGGTTAGTTAAATAA
GGGTAGCTTACCACTGAGAAATTTGGCTCTCCTGCCCCCCCCCCCAACAA
CCCTTAACTGCCTAGAACTTCCCAGAAAGGGGTGGGGCCTCATGAGCCCC
GCCCCCATCCATCCTTGATGGACTCACCAGCTCTGCCTCCTCACTGCAGG
ACTCCTGCAGGTGGCAACAGTCTTGAGTCTTGCAAGCATGTCTGTATGTC
TCCTGAGTCTCCCGGCTCACTGTAGATGTGCCACAGGTACAGTCAGAAAG
ACCAGGGCCCCCATCCCTAACTGACCACTTATGAGTTTTGTTCATTTAGG
TAAGGCAGCTTCCTTTCAGGCCTCGGTTTCTTCATCTGTATTTTAGGGAC
TGTATTGCTTTCAGGGCGGAACAGTAGAGCATGCCACGTGGAGGTGCCGC
TCAACTGACTTGGGAAATGCTATTTTTTTTTAAGACTTGCGCCATCTTTC
ATAAGCATTGGAGCATTTTTGCGGAACTTTCCTACTGCCTCTGCTAGCCC
ACCCACATAGATCCAGAAAACTGACGCTTGCCTTGATTGACTCTGTCGGC
TCTCTGACCTGTTCCAGATGCTTAACAATGGCCACATCCTGCCCTGCAGC
TCATCATTAAAATACACCCCCAACATTTCACATCAGTTAAAGTTAGTCTC
AACCTAGCGATGACCATGCACAAAATCCCTTGCACTGCCTGGGACACTTA
ACAGTAGCTCAGGGTTCGGTGAACTCCCTTAAGTGTCGCCTTAAGGATCA
GTAACCCATTCGTGCATGGCCTCCCTAAGCCCCTTGACCCCTGGAATGGC
TGCCAATCATTTGGTAAATTCATTAGTTGGTTCCTTAATAATTGCAGACA
CGCAATCCACACCTGCTTATTGAGTCTGTGGACTGCCAGGGTGGCCTCTT
AGAAAGCGCCGTCCTCGGAAGTCCTGCCTAATGCTCTGCTTCCTGGCCAA
GGGCCTCATTAAAGACTGATTTAACAGAGCAGTTTGGTAAGGACTTAGAT
ATTCCCAGGCATTCTCTGTTTCCCCCTTTGTCTTTCCTACATTTCCGATG
TGGTGTGTTTTGTTAGCTCTCTCGTCGCCCCGTGTAATTGCTGGTCCAGT
TTCTTCCCGGCTTTCTATCATTGAGCAGTAAGTCTGTCTTCAGCCCCGAT
TGCCCACAGCCTTAATTGATTCTGCCTAATCTCCTTGCAGGGGCCAAGGT
TAATTCCCCAGGCACCTGCCACTGAGTCAGCCTGCAGTAAATTCTCTCCC
CCACTGTAATCATGGCCCCAGTAACACCAGCCAGCAAATTTTTCCAGAGA
TTTGAGGTAACCGGCGCTTTATTGCCAGTCTGAAGCTTTGGCACTGAGCA
GGCTGGTTGTCACCAATCATATGTCAGCTGCATTTCCATCCATCTTCCAT
GACATCTTCTAGTCCCGTGAAGAGCAAGGACAGAGGCATGCCTGCTTGGT
TAGCTGCTGGTGGCCAATGTGACCATAGTGGCCATCCAAGCCTCTAGGGA
GATTCTCCAGGAGCTGGGTGGTCTTTCTTATTTTCTTCTTTTTCAGCAGT
ATTGGAGACTGAACCCAGAGTCTTATATATGCTAGCCAGCTAAGAACCCT
ATCACTGAGCTGCCTCTCCGTTAAGTTTTTGAGTCAGCATCTCTCTGAGT
TACCCAGGCTGATCTTCAACTCACTCGGCAACTCAGGCAGACCCTGTACT
TGTGACTTTCCTACCTCAGCCTCCAGAGTGCTAGAACTAAAGTACTCCAG
AGTAGAGAGAACTAAAGGCCTGCATCCGCCCAGAGCTGCCTTAAATAATT
TAATTATTGCCAGCACTGACTTTGTCACATAAGGCCTCTGAAAGGGACAC
CTAAACTCCCCCTCCACTGTGGCTACACTGGAGGAGGATACAAGCTCGAA
TTCTTTCTCGGATCATCTCTTCATTTCATTGTATATCACCTACAGTCCTA
CATATGAAAGCAGGATGTTGGTCTGCAAAAGTCCTGACATAGTTTGAAAG
CTCTCAGACTGTGTCTCAGGACTGCTGGACCCCAGGATTGTCTTTACTTT
AGCTGGCTGTGTGTCACCAAGTCAGGTAGCCTTTCTCTCAAAGCCACAGT
TAGACAGGAAACCTCTGAGATCATGCCTAGGAAGCCAGAGGTGGACAGGT
GTGTGGCAAGGATAGAGAGATACCTAAGTTTATAAGAAATCAAATCCAAG
TGTTGAGAGAGATGCCGCTGCTTATGCCCTGCTTAGAAATCACACGCCTT
CAACAAGATAATGAAAGAAAAGAACGTTTCATTTAGCATGGTCAAAGGCT
GCTGTGCGCAGTGTTCCTGCCTGCCCAAGCCCCTCACCTACACTCCCAGC
TTCTCACAGGCTATACCTGTATCACATAGCTCCTGTGGCAGGGCCTGGAC
CTTATCTGTATCAAACAACCATCAAGGTCTGGACTGTCAACTCCTCACTC
TGTACCCAGGGGACACACCACAGCAAGCTGGTATGCTCAGATCTTTGATA
AGTGACAAGGAAATGTGCTCATAGCTCCAGAAGCCAGCCAAGGTCACTAG
GTAATGGCACCTTCCATTTGCCCACCCCTCTACTCAGTGCATATTCATCA
GGCTGCAGAGGAAGGTCAAGTGTTCTTCAAAGCCTCAGATCATTGGTTGG
GTCCCCGAGCCCCAGCCCTGGCTGGGAATGGGCTGCTGCTTGCTCCTGAT
GTTCTGGGTGTCGCAAAAGGATTGCTCGTGAGTCCTGCATGCAAGCTGGC
TTTGCAATATCAGATGCACATCCTCCCTGTGCTAGTGCCCCAACAGTGGT
ACTGTCAGGACTCTCAGATGGAGAAGCAGGATTCTGCAGCCTAAGGTTGT
TACATAGACAGACGGCCTTGGGCTGTGTTTGGGGCTGGCTAGCCAAGCTA
CTCGGCAGGTAGAAACATGTCTCACCTGAGATCCCTTCTTCAGCACTTTT
TTGCCTCAGTCTGGTGTTTCACAGATCCTCCTGCTTCGCAAGCACAAGCT
CTTCTAAGGCAGCCACTGAGCCTTGTGTTGGAACAATCTCTCACTGTGGT
TCATTGTGTGTCATGTGCTGCCCCCCCCCCTTCACACACACACCCACGCC
CAGTGCCGGTTCTTGTGCTTGACACACCATTTGTACGGTCAGAGCTCTTT
CAATGATGTGAAGGGGTCACGGATTGTGGTCTGATCAGGAACCAGAGAGG
CTCGATAGATAGTTCTACAATCAGATAGATAGTTTTATGCCTGTGAAATA
TTTAATTTATATAAAAACTACGGATTACTGACAACACTTTACATTGTGAC
TTCTGTCCAATTCATCTTACACATAGTTGTCAGATAATAAAATAACCCCA
TGTCATACTCCAGCAATGCATAAACTTACAGCTGACTTCATTTTTCATCT
TAGTTCTGCCACAGGCAATCCCCACGGCCCTTTCTCCTTCTCCTCGGTCT
GTCAGAGGAGAATTGCCCCCTGGAGATCACTTAATGCCAACCATCCTTTT
TAGCCCATAATGAACCTGGCAAGGTCACTGCAGGTGACTGGTAGAGGTAG
CAATAGAGTTCTGATTTCCTGGCTGCGGTCAATGGCCACCCATTCCAGCT
TGGAGCAATATAGGTACATGTCACTCATCCTTTCTCCCTCCTATGGCTCG
TTCCTGGGCTGGGCAAGACCCTTCCCCACTTGCTTCTCCTGGGTGGAAGA
ATACCTCCCACTCACTGTTTAAAACAAGCTTCCCCCAGTTGGAGGAGACA
CAGTCTGCCTACAGGAGAGGCCAGTTCACTCCAGCACCCAGAGAAGATGT
GGATGCTAAATGCTCCCCACTCCTTCCTTGCTCCTGATGCTCTGAGCAGA
AAGCAAGCACCAGACAGCAATCCAGACCCCAGCGTTCCACTCACCGCTTG
CCCTTCCTGCGAAGTGAGCCATTTGCAAACACAAAGGTTCCCAGGACTTC
CCTTTGCCCTCCACACCAGGCTAAAATTTACATACACAGTGTTTTCGAGC
AACAATGGGGAATAATTCACTGAAGAATATAACCTAAGTTTTCCCAATTC
TATAACGAACACATAATCATATTTTAAAATGTTGTCGTGCTCCCTCCCCC
GCCCCGCCCTGCTTCTTTTTACAGTACTGGAGAGAGAGTCTTCGCTCTTG
CATGCAAGGCAAGCACTCTGGGCCTGACCTACAGCCCCCCCCCCAAAAAA
AAGTAAGATGCAGAGGAATTTAAATAAACACACCTATAACCCCATTTCCG
AGGGATCAAGAATCTCTCCCTACCCAACCCTGCCCTCGTTCTCCATGAAT
ATATATATTTACACTCAGAGAGACTGTCTGCCGTGGCTTCCTGCCTCAGA
AAGACCTTCTGTTCTGGGACTCATTTTGGACCGTCACCAGTAGAAAACCC
TACCACCAGGGTCTAGCAACTCTTGGTGTTTGTCAACAGAATGTCCCCAA
GTGTTCCTGAAATAACTTCACAGGGCCCAGCTGCTATAATATCGTGAGAT
TCCCTAGGGAACCCTGGAGACCTAACGCCTCTTTGGCACTGCGGACAGGA
GGGGAGGGATTTAATTTAATTGATCATATAGTTCCCAAATAAAAGACACA
AAACATTTATATTTAAAACAAGCTGTAACAGCACTAGAGCTGGGCAGATA
TCAGCCCTCTGTGTTATTTTGTCTACTTCCTTGTCAATAACCCAGAGATA
TCACTTGCTTTGTTCCCCCTGGGCTGCTCCTACTCCAACTGGCTGGCCCT
CATGGCCATGGGGTCATGATCAGCCTACCTCATGGTATCTTCTTCCTCCT
TCTCTCCTTGTGGTCTCTGCTTCAGACTCCAAGCCCAGAAACCAAAACCT
GCCTACCTCTCTTCTGCCCAGCTGCAGGCCGTAGGCGTCTTTATTGACCA
ATCAGGGATAACTTGGAGGGCAATGTTACATAGCGCCAATTGGGTGTACA
ATGAGGATTTCCTCGTCTCTGGAGGCAACCAGACGTTGGGGTTCAGTACT
CAGCGTTACAATATGTAACAACAGACCAAACCTCAACAGGGACCATCAAA
TGTCACCCCCTGAAAGGAGTCTCGGTGTGAGTCGCCTCAGCGTGGCTCTG
CAGGGAAAGGAGGGCTACAGCGGATGTGGTGACATACCACCATCGTTATC
TGCATGTGATGCAATAAGAGAAATGAGAATCTCTTCATTTTCATGCTACC
ACATTTGCCCCAGTGACCCAGCAAATGACGCATAAGTGTACGTCAGTGAC
ACTCAGTGTGGCCTTCTACTAAATGTGAGCTAAATACCCATCTCCATGGG
CAAGCCTCTCCCTACACTCACAGTCAGTATGTGATCTTCTGACCCCATTA
GTCTAAGACCCTGTTGTTTAATGGGACTGGAGATTCAGACCCAATGCATC
CTGAGAGTGACCAGGCCCGGCTACCTGATGTGGGCCTCAGAGAGATGTTC
AGGATCAGCACTGTGTGGAGGGTGGGTAGTTGTTGAGGTGCAGATGGGGT
TCCACTGACGAATCACTTTGGGAATCCAGGACTCAGTAGACTTGTGAATT
TATTATACCAGTTTTCACAAATGCAAGAAAAGTGTTGGGGCAGAGCAACT
CAGCACCAGGGCCAGCAGGCCCGGGGCCAGCAGCTCCATGTAGGTCAGAC
TGGTACTGTCCATTTGTCCATTGCCATGTTGGGGAAGAGCTGCTGAAGAG
AGAGGCCAGTGGGTGGTGGGGAAGGCAGGGCCCTCAGGGGACTTGCTGAT
TCAGCCTTTGGAGTGTTGGCCACAGCCCAGACCTGGTTTTATGAGGTCTC
TAAAAGTGACAGGCACTCCATCTCCCACAGTTCAACTTTCCCCAAAGTGA
TACCTGCATTGCTGCGTGCCATGGGGATACCCTGTCAAAAAGACAAGGCC
AGCCAGTCAACGTTTCCACTCCTCGAGGAGCAGGTAGAGACTTCAAAATG
GTGGCTGTCTCAGTCAGGGTTACTATTGCTGTGATGAGACGCCATGACCA
AAGCAACTTGGGGAGGAAAGAGTTTATGCAGCTTATGCTTCCACATCACA
GTTCATCATCGAAGGAAGTCAGGACAGGAATTCAAACAAGACAGGAACCT
GGGAGGCAGAAGCTGGTGCAGAGACCATGGAGGAGCCCTGCTTAGTGGCT
TGCTCCTAATGGCTTGCTCAGTCTGCATTTTATAGAACCCAAGATCACTA
GCCCAGGGGTGTCGCCACCCACAATGGCCTGGGTTCTCCCCTTATTAGTC
ATTTAGTAAGAAAATGCCCTTTGATCTTGCCTACAGCCCGATCCTATGGA
GGCATTTTCTCAATAGAGGCTCCATCCTCTGATGACTCTAGCTTGTGTCA
AGGTGATGCAAAACTGGCCAATGCAGAGGCCCAGGCAGGTAGTCAAGGAA
GGATATTAATGCACCATGGTGCTTTCTGTTCCTTCCTCATGGAGGCCAAA
GCTGATGGTCTGGTCTCCTGGCAGCCAGCCCATTCTCCTAGGTTGGTTCT
GGACCAGTTTGCCTGCCTTCCCAAGCCCATGCAGAGGGGAACAGCCAGGA
ATCAGAGCTCAGCTACCTCCTCTCTCATCACCAGCACCTGCCGGAGGTAT
GCTGGCTTCCCCGCCTCTGTGTGCAGCTCTTTCGCATTGGTTTTCTTCCA
AGAACATCTCTGTCTCCCAGCTCCCTGCATTCTCGAGGCCGTCCTCACTC
TTGTCCTGATTTTTCCTCCTGAGATGCTGTACATCGGATCATGGCAAGCA
GGCCCTGGGGAGACACTTCAGAAGTAAGCTGAAGAGTCTCAGGGGAATAG
CAAACAACAGGGCTGCAGTCAGTCTGCACGGGCAGGGCCTGCCAGCACTC
TGGATCCCTGTCCACACACCCCTAGCACTCAAGCCCCTTCTCAGACCCAT
GTCCCATCCCCAGGGTTGCTGGTGTGCAGTGTGTCACCGTGCCTGGCTAC
TCCAAGGGCTTTGGCTACCAGACCGGCCAAAGCTTCTCCGGAGAGTTCGA
CCACGCCTGGAACGCTGTGTACCTTGAGGGTCGTTGGCACCTGGTGGACA
GCACCTGGGGCAGCGGCCTGGTGGACACCACCACCTCCAAATTCACCTTC
CTGTAAGTACGAAGCTCAGTTTTGTCCGTGTCTTTCCTATCTGTGTGTGG
GGTGCAGACAAGGTTGAAGAAGGGAGGCATGTGGGGGAAGGGAAGGGCCA
AGTGTTAGAGGAAGACCGGACAGCCTTGCCTAGCTACTCTCGCCTTCCCA
AAAGCTGGTTCCAGGTCGCTTGCAAGGAGCTGAATTTTCTTGTCACCTCT
GCCCTCTGTGACTCCTCTGACTCTCCATGAGTCCATAAAGCTAAACCCAG
GAGCCCCTCCTGTTTGCCCCAGGTCACATATGAGGAGGGGCGAGGCTAAG
GTGCCTCCTTTGTGCTCCATCTTCTCCTCCAATTCCTGTTCTCTGAAGGA
GGAAGAAGGATGCAGTTGGCAGAAAGGAGAGGTGTAGGTGGGGCTAAGCC
ACCTGGGTCCTTACGGTGCAGTCTGGATCATCCTCTAAGGCTTGTTCAAC
CCTGAACTTAACCTTCAGCCAAGCATGGCATTTTCATGGGTCGTGATGTC
CCTCAGCAGCCTAGATCCCCCTTTATTTGGGAAACGAACTGTTTCCTCCA
ATGTGTGCAAGTGAAACTTTGGCCCTCCACACTCACCCTACATAGAGCTG
TTACCCTCAACAGTGCCCCCCTTAGGCGGAGCAAAGCCCCCCAAGCCCAG
GCTGTCTCCCTTTCCTTCTCTTTGTCTCTGTCTCTGACCAGCCCTAAGGA
GCCACAGAAGTTCCCCTGCTGCATACCCCCTTCCCCCTGGCACAGGGGCA
AGAAGGGTCCAGCGCAACTAGAACACTGTCCCCTTGCCTCCCTACCTCCC
CATCCTCTCGCTGTCCACACCCGCTCTACGGATCATGGTTTTGGTCCTTT
CAAACCACTTTTGGGCATTAAACAGAGAGAAAGCTGTCCTTTCTACCTCA
CCCTATCCATAACCTCCCAAGGCTCTGCGAGCAGGCCCTGAGTTCATGCT
GTGGCCTTGTCTCAGAAGGGACCTCAGATCCCCTGTGGCCCCAGGATCTG
CACACAGCCACCCGAAGAGTCTCCAGTGCCCGCATTAGGACCACAGATCG
TCCTGCCGACAAACTTCTGTTATCAGCAACACCCAATCCAGTGGTTGGGT
CAGCTAGACAAAGATTTCCCAAAGCTCCCCCGTGAGATACCCGGATGTGG
GTGATAATGGTCAGGCCTACTCTCACACCCGGCTCCTAACCAGGTCTTCC
TCTACTCCAGCTACAATGAATTCTACTTCCTCACCCACCCCGCCCTGTTC
ATCGAGGATCACTTCCCAGACAACAAAAACTGGCAGCTGCTGAAGCCGCC
CCAGTCTCTGAGGCAGTTTGAGAACAGCATGTACCACAAGAGTGAATTCT
ACAACAAGGGCATGCTGAGCGCCCACCCGGAGACGTCCATGATCAGAACA
GGTAAGGAGAAGCACAAGGCGGAGCCCCAGTGACGGCTGAAGCTCCCTTC
CCACTAGAGTAAGCCAAGAAAATCAGACCACCAAGCTTAGCTATCTTCTA
TGGCCTGAGCTGACACTCATCGCCCCTTGACACAGCATGGAAACCATGGC
TGCTGGGCGTTTGCAACACTCTTAAGGGGCAAGGCCTGACCCCAGGACCA
GACCCCAGGCAGAGAGGATAAAGAAGTGTCATGGACCTCAAAGGACTCCC
CCTAAAGGACTGTTTCTCATGCCTCTCTGGGGATCCAGAGCCTGGTCCTG
CTCTTGGCGCACCACAGCAGGGGTCAGGAAGGCTGACCAGCCCACCTCCC
CCTTCTCATCAAAGCCCAACCTGACAGCACCAGCTCTGCCACCAGGAGGC
CTCCACACATCCCAGACAGAAGGCCTGAGATCCCAGAAGCAATCAACTGG
GGGGGGGGGGTGGGCATGAGGGATGATGGCCTTGACCCCACCCCACCCCA
CCCCACCCCACAGGCTGATGGGATAAGGGGACAAGGCAGGGGGGTCCACG
AGGGGGCAGTCAGGAGCAGGTACCTCTTGCCCATTGCACTTCCCATGGAT
TCTTGATCTGGATGCAGAGCTGCAGTGGTCTCTGGTTAGTCACAGACACG
TGGCCGCTTCAGCAGCCCACAGCTGCTCACTTCAAAGATCATTTCCTGCA
CCTGAGTCTGGCAGAGGTCTGCGGTCTCTATCGTGAGACATGAAGCCACA
AGAAAGACTCGAGCAGACCACAGTGCCCTCGTGATCATACTGCTGAGTTG
AGTTTGCCTTTTTTTGGGAGATGTAAAAATGTAAACACCAGATTTCTTAA
TTTATACTAACCTAAAACTCAATTCCATATTCATGCTCCTTATTGTAAGA
ATATTTCTTGGTGCCTGTGAGTTGCCTTGAGAATAGAGCTTGAAAAGAAG
CAAGGGAAGGGGGCAGGGCGTGAGGGTAGGGTGAAGAGGAAGGAGAGAGT
CACAGATACAGGGAGGAGAGAAGAGGCAGAGAGAATAAAGAAGTGCCAGC
CACAGTCCTTCAGTGGCCTTTGGGGTCCCCAATAATTCACCTCTTCTAAG
ATGCTCAGCACACTTCTGCCAAATGTTCTCTAAGACAAGTGCTAAGCTCT
TCCAAAGAGCCTACAGCCCAGACAAAGGCAGAACATAGAAAAAACACTCC
CATGATTCATTCTGGAAAATGGTGGGTTCTCTGGAAGAGGCCTCCAGCAT
CCCTCCAGCACCCCTCCAGCATCCATCCCTCCAGCATTCCTCCAGCACCC
CTCCAGCATCCATCCCTCCAGCATCCCTCCAGCATCCCTCCAGCATCCCT
CCAGCATCCCTCCAGCATCCCTCCAGCATCCCTCCAGCATCCCTCCAGCA
TCCCACCAGCATCCCTCCAGCATCCCACCAGCATCCCTCCAGCATGTCCA
GCTCACCTGAAGGGCTGAAGCAGACTCTTTGCAGCCAGGTCTTTAGGGTG
AGTCCCCACCATACATGGAATGGAGGATTAATAGTGGACGTGAACCAGAC
AGCCTTCTCCTTTGCCTTTCTCCATCCATGAGCGGGACTGGAGAAGTTAC
TGGGCAAGGGCTGGGGTGGTCAGCTTCTAACAGAAGCCATGGCCGGGCTC
TCAGAGCAGACACTGCCTGGGGAGCCAGACAAGCCCAGATAGAAAAAGCC
TTCCTGAGCCAGTGAAAAGCAATGTTCCAGTAGAGGAGTACAGAGGCAAG
GCAAGGGGGAGCTGGCAGGAGAGGGAATGGATTCAGTGGGTTCGCTGAGA
CCCAGGTCCAGAAGCACAGCTCTGGCTCCCTGTGTGAGAAGCAAGTGTTG
GGAGGGGAGTCCAGAAAAATACCCTGTTAACACTCCTGGGCTTAGACTCC
TGGAGGAAAGCCCGTTCTGGTTATGGTGTGGTGTTCATATGACAGCTGTT
GATACCATAATCTTTTCAGGGGGGTGATACAAAAGTAATACTTCAGAGTT
GTGGGGGGTGGGGTGGGAGGTGGTGGATGGATAGCTCAGTGTTTAAGAGA
GCTTGGAGCTCATCCAGACGACCTGAGTTCTGGTACCCACATCCTTAAGT
CAGCTCACAATCGCCTGTAACTCCATATTCAGGGGGATCAGTACCCTCTT
CTGAACCATTACACACACACACACATACACACACACACATACACACACAC
ACACACACACACACATACACACACACACACACACACGCACACACAGCATT
CACACAAAGACATATGTGCACACACATAAAAATAAATCTCTTTTTAAGAC
GTAAGAATTGAATGAGACAGCACTCTTCCAATACTTTTAGGCCCCTCCCT
GCTCTGGAATCAAATAAAATATCTAGGCAGGCTTCATCTAATGTAAAGCA
AGTGGGAGAGTAGAGGAAGGGAACACATATGGCTAGGGTAGGGTTTTCTT
GCCCATCCTCCACCCCACCCCACCCTCCCCAGTTATCACAGTTATCAGCT
GTCTCAAAAATATTCACCCAAATGAGTCAGAAGAATACCGCGTTCAGGGC
TGTCACACTGTTAAAAATCAAGCTTGGTAGAAATCTTCACAACCACGGAC
ATAGACACACACTGTAAGTCACACCTCCCCCCCAGTGCTTAAGCATCCCC
CAGCACACACTGGATCCGGCTCACCTAACCACAACTTCCTCTCCGCAGTC
AATGGGAAAGCCACAATCACCATCGAGAGCCGGGCCCCAACACTGTTCAT
GTTCATGCTCAATGGCAAGCAGGAGCACGGGCTCCTGAGCCTCAGGAAGA
ACGGGATGAAGCTGGAGGTGTACCCTCCAACCATGGGCACCCACAAGCTG
CAGATCTTCGCCAAGGGCAACTCTGAAATCTACAGCTCGGTGCTAGAATA
CACACTCAAGTGCAACTATGTAGACTTCTCTGTCCAGCTGCCCAGTGAGC
TCCACCAGCCCGTGGGCCCCAGCTGGTTCTCGGAGCAGATGGGCATCACG
AAGCCCTCTCACTCTGACCCTATCATTCACACCAGCGATGGCCGCTGCGC
CATCAGCTTTAGCGTGGAAGAGGGCGTCAGTGTCCTGGCATCCCTTCATG
GCGATGACGGCCCCATCACTGAGGAGACACAGCGGCGCTACATCTTCCAG
TTGAACCGAGGGAAGAGGACCGAGCTCAAAGTCCAGCTGCCCCATGCCGG
CAAGTTTGCCCTTAAGATCTTTGTGAAGAAGAGGCAGGAACAAGGGAACT
TCATCTTTGTCTTCAACTACCTCTTGTGCTGCGCCAACACCAAGGTGAAC
TGGCCCATGTTCCCTGAGAGCTTTGGCAACTGGGGACAGGACAATGAACT
GCTAGAGCCTCTCTCCGGTGTGCTTCCCGCCAACCGCAACATAGCCTTCA
AACTAAAGCTGCATGGTATCGCCAAAGCTCTGGTGAAAGGACAGGACACG
TGGCCCCTCACTCTTAACCCCGAGGGCTACTGGGAGGGCAGCTGCAACAC
AGCTGGCTGCCAGGAAGTCTACGTCATGGTGCTAGAGAATGCTAACCACA
ACTTCTACAGCTACATCCTGAAATACAAAGTGAATGACCAGTGAGGGGCT
GCATTTCTTCACGGTCCCCCAGGGTTAAGGCCAGCCCTGCCCAGGGAGGG
CCCAAGCTAAGAGCAGAGAGCCCAGGACACCACTAATCTGCATGAAACCA
CTTCTAGGCCTCTGCTTCAGTCCCCTCTGCCTCAATCTCCCTGCTGTGTC
ACAGGCACTGAGGAATTTGTGCCGCCAAAGGCTCACTCAATGTGGCTGTG
GAAGAAGAGACAGAGGCCAGGGCCCTCTAGAAGAAAAAGGTGCTATGTAA
ATCCAAGGTATTGTGAAACGTACACCCCTGCCTGTGCCTGCTCTTGTGTT
AGGACTTTGTTGCTGTAGAGCTGAATGGGAAAGCGGAGGTGTTGCGGGTG
CTGAAAGCTTTCCCCACGATGCCTGCCTGGTGCTTGAGGCTGAATCTATG
AGAAAGGTGTGCTCCCCCTGCAAGTAACATGGTGTTTCTCCCCACTACTT
CTGGACCCCAGGGAATGTCGGGCCTGCCCATCTTCCACATTCCTAGTGCT
CCTATTAGCTACTTCATGCTCTTCAAGAGCTTGCAAAGGGGGTGGGTGTA
GAAAGCAAACCAGCTGACCCTCAGCCAACTGGAGGCTGCTGTCTCCTGAT
GCCTAGCATCTCTGAGATGTGCTAAGGGCAGGAAAGCAGCCGTGTGCACA
TTCGGATGTCCTGGCTGGGGACCAGGGATGGAGTGGGTTTGCAGGTCCAG
CAACCCACATGGAAGGGTGAAGACCCCGATTCTATGTGCCTACCAGAGTT
TAGCTGAGGGCCTGGAGATTGCCCCACCCCACCCCCCACCAGCCATCCTC
AGGAGCCTCAAATGCCTACATAGCCCACTGGTAGCCTGGCTGATGACCTG
GGCCCTGGGCAAGCAGATTAGCCCCAGCAGTTATCAGATACCAGGGATAG
TGTCTGATAACAAGATGTTGCTCCTTCCTTACAAGCCAAACCAAGCTGAG
GGGACCACCACCTGCCCCCTAGAGAAAGTGTTGGTCTCTGTCTCTGAGGA
AAGCCCTATCATAGCCATTTTCCCTCCTGTCTTTCTCTGAGATGACAACC
CCTGCCCTGAGTTCAACCTTCCTTCCATTGGGAACAGTTGAGGGCCTCTT
ACTTGCTCCGGCCTGTCCTAAAAAGCCTGAGCTGCCTAGCTGGCCCTCAG
CAGCACCTGAGGCTGGTGTTTCCATGGCAACAGCTGAGGGGGGAAAAGCA
GGCTGCTGCAAACAGGGAAGGCCTGAGGAGAGCCCCCCGACAAGAGGTTC
CTGCAGTAGCATCCCAGTGCACAGATGTGCTCCAGGAGGGTCCCTACCAA
ATTTCAGGGTTCTAGGACTCTGCTCACTGTGCCTGTCTCAGGACCACCCC
CCCCCCCAATCCCTCACTGCTCTCTAAGGAATGTCCTCAGGCTCAGCTGC
CGCCAGAGCCAGCCTGGGATACTCACAGCCTCTACAGTCAAGTCTTTCCT
CAGTAAGAGCCCATCAGACTCCTTCGATAAGTGGCTGACCCCTCATGCTA
GATCCTGGGACTATAGAGGTGAACACAAAGGACTCAGCCATAGGCACCCG
CATCTCTGGGTGGGCAGTCTGCCAAGTGGAACCTATCTGAAGACCTAGGA
GCCCAGAACTTCAGCTCTTCATTCCCTTCTGCCCCTTTCCCACAGGTCCC
CACTGCTACCCACCTGATACCACATAGCCCCCCTCCACCAGACTGGGTAG
CCTCACTTGTGATTAGCATGGCTGCTAGGGGCTGAGCTGCCTTGGCTCAT
GTCCAGGGTCCTCTGGCAGTCTCAGCCCATCAGCATCTTGTCACTCACGC
CTGGAAAACGCACCAAAGTGGGGTGGGGTGGGGTGGGAGGTGGCGTTGTC
TCCACTACAAGAAGACGGTGGAGTTTGCACTGCCTGGAAACAGCCTTTCC
TCACCCCTTTTATTTTATGAGCAGTAAGAACTGTGCTATGTTTTAACGAA
ATAGTAAGTGCTTCGGCGGTCCCTGCCACTCGCTAGACACAGCCGCGACA
GCCACTGCTAGCCCTGCAGAAAATGATCCCCATGTTTTTTTCAAAACAGC
ATAATTAATCTATTCTGGAGCTTGTGGAGGATGCTACCCCACAAAACAGG
AGCTGTCCTCAGCACCAGAGACCCAGACCGAGCCTCTCCCAGCGGCCCGG
GTCAACCACACGCTCCGCCCTCCTCATATAACCAGGTAGTCCCTTTGTCA
AGGCAACTGTAGGTGCACTCGGAAAAACGCCTGCGTTTCTTTTTACTGAA
GCAAAGATACACGAAACCGAATCCAATCTTCATGAATTTTTACACGCATG
CTGCTCAGCCTGAAAATTTGGAGGCTGCGTTTTGAAGGCTTTTTATTCAA
ATCCCCCTCTTTGTCTGCTGGTCCACGAGTCTCATTGGCCGATCAAATGG
CACGAGCGCCCCCTGGTGGATCACGTGCGAAGTTGCCACCGCTGCAAACC
CCCAAGCTGGCTCTTTTTATGATGTTTGTTAAAAATCACTAGGTGCTTTC
TCGTCCCTTGCGGACAAGTTATGCTGTCTACGGCATCCACATCGTGGATA
GTTATCCTCGTGGTTGATATGCCAATGTCAGCGTTGTCTTGCAGACTGTC
AGAAGAGCCTGGGGAGACTTCCCTGGCCTGGCGTGGTGAGAAGATAGGAT
GGGATTAAGAATTGAGCATCTTGCCCAGAAAGCTTTTCCTGAGACAGAAA
CCCTGGGCACAGGAATGCCTGTCTGGGGTCGCTGTGATCACACTCATTAC
TATGGCAATGACAGGCTGAAACTGATCAAACTCAGCGTTTTGCCAGGCAG
GCCCCAGGATGGGCGCCAGGGTGCAGAAGGATGGGGCGTGGTGAGGCAAA
AGGGTGCAATTAAGCTCTTTCAGCTTGCTTCAAAAGTGCCACCAGCTCAG
CTGGACTGTTCTCTGTCTGATTGTGAATATTTTGAGTTCTCCATATGTTA
CATTGTCCCACGAGGACGGACTCCTCCCGGCCCACAGAGATTAAAGAGAC
GTAAATAGATTAACTGAAGCATGAAGGATTTGAGCTAGATATAAAGAACC
ATTTCCTGTTGTGAAGCAGGAGGGACACGTTGCCAATAAACTCTCGGCGA
AATTCTCAAAGGACTTGAAAAGGAGAAAAGTTCCCGCAGTGGTCCGGCAT
TTTCTTTGCCGTGCTTACTGAAGCGGTTGGGTTCTCCCTCCGCTAACTCG
TAACTCTTAAACAGTACTGGTTGGCCGGCAGTAAACACCAGGTTAATGGT
GTGACCATATAGCTCACTGTTGTGGGATTTCAAAGGCCGTTTATCTGAGG
TCTGCCATTTCCTTTCATCTGGAGTAAGTAAAGCCGCAGAGGTGACTATT
TCTCCCCGTGTGTGCTGAGTAGTGTGGGCCACTGGCTGTTAAAGATGCTG
TCCTCACATTGTTGCTGGGGTTCTTTTTCAATAAAATTTATATTCATTTT
TA

Cep63, Centrosomal protein of 63 kDa, is a protein that is required for normal spindle assembly. Cep63 plays a role in the DNA damage response. Following DNA double strand breaks, Cep63 is delocalized from centrosomes, leading to inactivation of spindle assembly and delay of mitotic progression. The cDNA sequence of Cep63 in Mus musculus is provided in GenBank Accession No. BC048718.1 (GI:28913748), incorporated herein by reference. The amino acid sequence of Cep63 in Mus musculus is provided in GenBank Accession No. AAH48718.1 (GI:28913749), incorporated herein by reference. The cDNA sequence of Cep63 in Homo sapiens is provided in GenBank Accession No. BC014050.2 (GI:33878420), incorporated herein by reference. The amino acid sequence of Cep63 in Homo sapiens is provided in GenBank Accession No. AAH14050.1 (GI:15559363), incorporated herein by reference.

An exemplary DNA sequence of the Cep63 gene in the Mus musculus strain C57BL/6J chromosome 9, GRCm38.p1 C57BL/6J is provided directly below.

NC_000075.6 (GI: 372099101: c102626124-102586578):
(SEQ ID NO: 2)
AACCGACCGCATGCCCCAAAGACGGGCGAGCTGCGGCTAACTGTGGTGCGGTCTCGGCCCTGGCCACGGTG
GTGCCGCTAGGCTCCAGGCCTCATGGCCCGCGGCCGCAGCTCGTTGCGGCCCACCTGCCGCGGGGCTTTCC
AATGCCGGGTTGCGGCTGCCCGGTCGTCGCGCCCCTGGGTTCCTCCTCCTCCCTATGGGACTCTGGGCTGG
CCGGCCAGGTGCTTCGGCCTGCGGGAGGGCGGGCTGCGGGCTGTGGGCTGCATGGCTCCCGGGAAGGGACC
CGGCTGCTGCTGCTGCCGCCGGTGTAGGACCAGGCCTTAGCGTTAGCCGCCCAGCGGGAAGCCCGCAGGGT
TCCTTGGTGATGTTAGTATGAGAGGTGCGGAAATGTGGCGGGAACCCCTGATGGCAGGGGAATTGCAGATG
GCTTCACGCTTTCAAGTCAAGCATAGGAAACAAGATTGGTGGAGTGAAAGTTTGGAAAGGGGAATTCCACA
ACAGCGCTTTCACGCGGAGACACTTGACAGTGAATGGGCTTTTGTCCTTTACAAAATCGTGGAGTTAGTGC
ATTCTGATGTCAGACCTAGTTTTCATTTACAGACTGTAGACTTGTTGCCAAAAGTGCTCCCCATTCTCCCC
AACTAGCGCTGGCTCTCCTTTGTAGCTATGATCTGGAACTTGCAGTGTAGACCAGGCTGGCCTCAAACTCA
GAGATGGGCCTACCTATGTCTCCTAATTAAGGATCCGCGCCACCTCGCCTGGCTTTCTTGAGGCCAATTTA
GAGTTTCAAAAATGTATTACTTTATTATAGCTCTTTGTACAACTCGAATATTTGTATTTTATATTTTGTAT
GTAAGTCAGAGATCAATGGCTATAGTTATCTTTAGCTTTTTACACTTTTTTTTTTTGTTTTGTTTTTCGAG
ACAGGGTTTCTCTGTGTAGCCCTGGCTGTTCTGGAACTCACTCTGTAGACCAGGCTGTCCTCGAACTCAGA
AATCCGCCTGCCTCTGCCTCCCGAGTGCTGGGATTAAAGGCGTGCGCCACCACGCCCGGCTTTTACACTTA
TTCTTAAAGGCTGCAGCTTAACGATATGATGAAACGAATTATCTTATTTGGAATGCAGCGCCATTTTTAGG
TTAATTTTTGTCCCGTGCTCTCTGTCTTTTACTGTGTCGTCCTCAGTGGTCAGTATAGTAAAGGAGGCTGC
GAAACTAAAGAGGGTTAGAGACTTAAACCACAGACACATGTCCCTTTAAAATGTGTATATGCTGAAGTTTC
AGAAACAATGGAGATATTGTTAATCATGCTGCTGCTGCTTCCTCCCGTGGAGCGGTAGGGACCGGTTACTT
AGGCTTTCCTGTACTGTGTGGTGGCTGATGGTGACAGCATTAGTCCACACTGTCATAGCTTAAGGTGCTCC
CCATGCCAAAGGCTAGAGTTGCTTTTTTAAGTTTTACAAAAACCACAGTACTATTTTTACATCAGTACAAA
TTTATAAAAAGCACACAGTGTCAGTGTCCGGTGAGTTCAGGTTCTTCAGCTATTCCATATGTGCTTGTAAT
ACCAAAGTTGTAGACTTGTAGACCAAAGTCCTACAGAGAACTGTCAGCTGCATTCGGTTGCTAGGCCTTCA
AAACCCTCTAATCAATAATCAAAACCCTCAATTTCTTTCTTTCATATATATACATATATATTATATACATG
TATTATATGTGTATATATGATATTTTCTTTATTTACATTTCAAATGTTATCCCCTTTCCTAGTTTCCCCTC
TGAAAATCCCCTATTTCTCCCCCCCCCCCAAGCAGGTTCTTACCTCCCGTTTCTTTATCACTCAGGTTTAG
CTGGTTGTATATATTTTTGAATTTATGCCTCTACCTCTTCTATGACCTCTCCTTCACTGGCTAAATCTTGA
ACTACACTATCCAGTGTGGCAGTCATCAGCCAGCCCTGACATTTTAAATGAACTGTTTGGTTCCCCAGATG
CATTAGCCACATTGGAAATGCCCAGCACACACAGGCTGCCTAGTGGCTATTGTCTGATTAATACTGATAAG
ATGTTTCCATAATTACAGAAAGTTCTGTTGCAATACAATGATCTTTTGAAATACAGTGTTAGATTCAGCCT
TGATTTTTGATGGCAGGTACAAGAAAACTCAAAGGTATAGGTTTTATATTATAAAAATTATCTGTTGCGTT
CATTGTCAGGGAGACTTGTAATGTCTGAGTCACCCCACTAGGGATGTTAGAATTAAGCTGTGTATGTCGGG
GTTCCTGTGCTGTTTGCTCAGTTGAGCCACCATGAAATTGCCCAAGAGCCTTTGGTCAATAGGGAGCTCTT
CATGGCTAATAGTGCCTGGGCCGATTACTGAGTGAGAGCCACCGTGGTGCTTTCTAGTGGCGTCATGTGCC
CAGTGCTGCTTAGCTGGCACTCTTATGATGAACTTGCCTTCCGCAGCTGACTATCCTGAAGTACAGTTTGT
ACAGGAATGGCAAGCAAAATGCTTAACTCCGTTCCTTTCTTTTCAGACTACAGCAATGAATTGGTGGCCTA
GCAACCTCCAAAGGTAACCCAAGGGCTCCACCTTAAAACAAACAGATAGACTTGAATGTGTTTAAATCTGT
GACGTAGCAACCTCCAAAAGTAACCCAAGGGCTTTATCTTAAAAACCAACCAACCAACCAACCAATAGGCT
TCTATGTGTTTACATTGTTTGAACATATAACAATTTTTTAAAAAGATTTATTTATTATATGTAAGTACACT
GTAGCTGTCTTCAGACACTACAGAAGAGGGCATCAGATCTCATCACAGATGGCTGTGAGCCACCATGTGGT
TGCTGGAAATTGAACTCACGACCTTCAGAAGAGCAGTCAGCACTCTTAATCGCTGAGCCATCTCTCCAGCC
CCAACATTTTTTTGATACCTGAAATTGTATCATTTCCTCCTAGTTTTGCTTTAGCTGGGTTTTTTTGTAGT
CAAGGATGACCTTGAACTTAAGATCTTCCTGCTTCACCTCCCCAGTGCTAGGATCCCGGTCAGTGCTGCTG
CTTTCCTCTCTGTGTGGTAGTTAGAAAGCACTGTCATCGTCCCTGTAAAAGAGCAAACCTGCTCTGTAGAA
AGTTAGCTTTATCCCTGACTCCCCCACCTGATCACTTCCCTCCGTAAAGGCAGTAGTTCTCAGTTTGTTCT
TCAGTAAATAAGCACCCAAAGGCGAACACACTTCTTCTTCTTGCTTTGTAGCTGTTTTCACCTTATCACAG
GCCCCTCCACAGATGTATGCTCTTTTGACAGCTTCCTAGTATTCCCTCAGTGAATGAGGGCCCTGTTGGCC
CTATGTTAGTATTTGACCTTAGTGCTAAGCGGTGGCAGATCATCAAACACTTTTTGCTTAAGTCTGTATTG
AGTATTAGGAACACAGAATTCTCAGCCACAGTCACGTGGCACCATCAAAGAGGAAACAAGACTTATTGAGT
AAATGCAGGCCATGTTGGTTAGGGACAGAGTCACTGATGTGGCTGTGATAAATATCTACAGGAAATCTCAG
TCCTCCCTGTGTATTGGATCCTAGAGAAGGGTAGAGTGTTTAAGTTAAAGAAACACACTCCACATTAGCCG
ACGTTTTTCAAGGCATGTTACTGGTCATCTGTGCCTGTTTTAAACTAGAATTCCTTTGTCTCAATACTAGG
ATTTAAGTTTTGAGCTTTTCTTATAGCTGAGTAGTTTTATCACAGTGCTGCTGCCACCTAGTGGTGATTAG
GCAATATGCTTCAGCTTTATTCTGTTGCCCTCTGAGATCCTGTTAACCATAAGAAGGCAGTGTTAACCAAA
AGATGGCACATTGCTTGGGTCTGGAGGGTCACATTATCTGGGTTTGAACTTAAGGAATATAACCTATAGCT
TTTTATAATCAAGCCGTTCTCCCTGTTTCATCAAAGGGGTAGCACCTGCCTTTTACAAGGAAGATAGAACG
TGTAAGAGGCAGGTAGATAAAGTGAACATTCACTAAAGTTTAGCATATATTATACACGTTGTTAGTTATTA
AATAGTGTCATACATAAATGCCAAGAAATTATATATTATTAAATAATCACTGTTTAAGAATGGCATGACCT
GCTGGGCGGTGGTGGCGCACGCCTTTAATCCCAGCACTTGGGAGGCAGAGAGGCAGGTGGGTTTCTGAGTT
CGAGGCCAGCCTGATCTACAGAGTGAGTTCCAGGACAGCCAGGGCTACACAAAGAAACTCTATCTCAAAAA
AAAAACCAAAAAACAAAAAACAAAAAAGGCATGACCTAAAGAGGTGTATTTTTAGTAGGAATTATTTTGAG
AAATAATTATAAAAAAGATGAAATAAAGACCTTGGCAGTTACTATTCATACCCTTCATTTTGCAGATGGAA
AAGCCGAAGTTCGAGGACACAGAGCTGGTTAGTTGCAGAGCAGAATTGTTTTCTGGGCGTCTGTCTAGTGT
TGGCTCAACTGCTTCCCAGTTCTGGAGCAGAGCAAAGGCTGGGTTGGGTTGCTCTGTAATTTGACCCTTAC
TGTGGTTTAGAAGGAAGTGTAGTAAAACGTGGAGTCCTCTCCGTTAGGATTTTACTCTGGGTCATGGTGAG
CTCTATCAGGAACACTGGTTGGAGGAAAGAGCCTTAGCCACCCTGTGCAAGAAGAGCCAGCAGTCCTTGCT
AGGACATGTAGAGATAGGTGCAGTTACAACACACTAGCTGTCCCTCTGGTCTTCTCAGCCTTTCTGTGTCT
CTAGAATCATCTGTGTGTCTTCTGTCTGTGCATTCATTGCCAGGGGATGTTGACCATTTTGTTTCTTTTTT
AGTCCCACATTGGGCTATTTCTTGGTAAGGAGTATGTCTTTTATTATTATGTAGAGGGCTGTTTGTACAAC
ACTAAACTGTTCAATAAGGTAATCTACTTCTCAGAGAAGTAGATTACTTCTGGAAAAGCATTAGGACAGAG
GGACCCAATCTCTACATCGCAACTTTTGGCCTTGTCTTTAATTCAGAATTAAATTGAAACTTTGAAAACTG
AGGAAAAAGGACTGTTTCTTTTTTCTTTTTAAATTTTATTTTATGTACATGAGAGTTTTGCTTACATGTTT
ATCTCTGCACTATAAACGTAACTTGTGATCCCCTGAAACTAAGTTTATGGAGAGTTATGAGCTGTCATATG
GGTGCTGGGAATCAAACCTGGGTCCTCTGCAAGAATAACAGGCCTTCTTTCCAGCCTACTGGGGCGCTTTC
TTCAGAATGAAAGATGGTCTCTGAAAGAACACATTGAGGATTTCTGAAGAAGAGAAATTTATCATACAACT
CTCGATACTTATGAGTTTAAGGGTTATTCTTTCTTTATGGTGTGAATTTCTCTGTTTAGGCAGTCTTTTCT
GTCTCCTTTCATTTTCCTTCGAGATCCTTCTTGGCAGTCCTGCATCTGTGAGCAGTTTGCTTAAGGGATGA
TGAAGTTTCCCAAGGCATGCAGCAGTGCCCTGTTAAAATCTGGTCTCAGCTCTGGACCTCAGAGATGAGTG
ACATTGGTTTTCTGTCACTGAGAAACCAATAGCTTAGAAAGAAGGAGGAATATACTAAACCTTTATTTTCC
AGTTCTATATAATTTGGCATGAAGAGTGGGGGACATGTCAGCAGCAGTAATTGTTGTGTTAGTGAGAAGTG
CTGCTTTCCCTTCAGCAGTGGTCCTGGGTGATTCCTCTCAATGTGTAGCAGGGTTTGTGTTAATGTCCCTT
GAGAGAAAATAACTTTTCACCCTGACATTCTTTAAGAACTAAGCATTAGAGGGGCTAGAAGGAGGGCTCAG
ACAGTAAGAGCATATACTATTCTTGAAGAGAACCAGAATTTGGTTCCTAGATCTACATCTGGTGGCTCCAT
TGCCTCTGGCTTCCATGGACCATACTTAAAAAATAAGTCTTGACTACAACAATAGAAGGAAAGCCAGGCCT
GGTGGCAAACACCTTTAATTCTATCACTCAGAAGGCAGCTGGACATCTCTGACTTCAAAGCCAGCCTGGTC
TACATGCATAACTGGTTCTAGACCAGCCAGGGCTACATAGTGAGACATATTGTGTCTAAAAAATTAAGTTA
AACAATTAAAAAAAAAAGCATAGAGTAATGTGATGTTTCATTGGTTTTGTAGGATAGTTTCAGCAGCAGTT
AGGATTTTGTTTGCTTTTGGAATGTATTTGGTGCTAGCATTGCTAAATGATAATCCTTGCTTCTACAGACT
TCTTAGGCTGCTTTGTATTTCTCACTGTGATTGCTTTTACTCACCCTAACTTGGAATCCTGGAATTCAGAA
CAGTAATTTTTCTCACTCCTCTTTTCTTTTTCTTTTTTCCCCTAGTTGCCAAAACAAAGAGGATTTAGAGA
TGGAGGCTTTGTTGGAAGGAATACAAAATCGGGGGCATAGTGGGTAAGTTTGCCTTTGACTGTGAGCTCAT
TGCAGTTTAAAAACCTACCTGGGTTCTGTTAGACTAAAGCTAAGCTGAGCTAAGGCACATTTGTGGACAGC
ATCCCTCCGACATCAGTTGCCTTCTCTCAGCATTACTAGGGAGGACTGTGGCTCCTATCCTAGCTTGAGTT
TTCCCCTTCAGCTCAGATGGGTATCAGTAGCTTGAGCAGTGAGGTAGGTTACAGTGTGTTCCAAATAACAG
TTGGGAGAAATAACAGCAATAGATCTTGCTCAAGGGTGAGTTTCTTGAAACAGCTCTTCTTTGTTATCTTT
TTTCCTGCATAATATAGCCTGTTTGCTACTAAGACTGTTCATTTTGGCTGAAAGAGCTGCAACACAGGCTT
GCCTGGATTCTCCCAAAAGAATCCCTTAAATATCCCAGAAGATGTGGTTAGTAGCTCTGAAATCTCACTTT
GCAAGCCAGATGTAAAGCATCCATCTGTGTTTCAGCAAGTGAGCCTGGTTTTAGGTTACTCATTAGAGGAG
GGCAAAATATTGTGTGGAGTTAAGCAGCATAGCATATGACAGCAAAGGCCCAGGCACCCTGGGCTTGGCTT
CTGTAATGTTTCAGTTCTCCAGATAGGTTTAGTCTGTGATATTTTTATCTGTGAACTAGGAGAAACCACTT
GGGCATGAGCTGTGACTGGTCTGTTGGTGCTTGTAGGGGATTTTTGACATCCTGTGAAGCAGAACTGCAAG
AGCTCATGAAACAGATCGATATAATGGTGGCTCATAAGAAGTCGGAGTGGGAGGGGCAGACGCACGCTCTG
GAGACTTGCTTGGACATCCGTGATCGGGAGCTGAAGGCTCTGAGGAGTCAGCTGGACATGAAGCACAAAGA
GGTAGAGCGGCTGCTTCACTTCTCTTGTTATCTTGACTCTGGCTTTAAATCATACATGGTTCCATACACCC
ATGTTCATGCATGTATATAATATACTTGGAGCAACCCATGTCCAAACCTTTAATCCATCTTTGTGGTAGTT
TAATCTTTTATGCTAAAGGTGTTTCTTAAGTGTAAGCATTTGTTTACCAAGTTAGGTTTGTTTGTTTGTTT
GTTTGGGGTGTGTGTGTGTGTGTATGTATGTATGTGTCTTCAAGACAGGGCTTCTCTGTAGCTCTGGCTGT
TCTGGTACTTGCTCTGTAGACCAGGCTAACTTGGAACTCAGAGATCTGCCTGTCTCTGCCTCCTGAGTACT
GAGATTAAAGGTGTGCGCCACAACCGCCCAATCTTATTCTAATTGTTACCTTTTCTTCTTTTTTTCTTTTC
TCTTCTTTTCTTTTCTCTTCTTTTCTTTTCTTGTTTTAAGATTTATTTTATGTGAATGAGTATTTTGCCTG
CATGCACACATATGTGCACCATATATATGCCTGATGTCACAGATTCTCTAGAACTGGAGTTAGAGACAGTT
GTGAGCTGCCATGTGGAGTACTGGGAATCCAACCTGGGTCCTCTAGAGGAGCAGTCCGTGCTTCTAACCAC
TGAACCATTTCTCCAGTCCTCTTAGGGTTTCTTTTAGAAGACATTTTATTATGGAAGTATAAGAACATTAG
TATTTGTTTCAGGGAGCATTTTACTTTGGAAACATATAATGCACACTGTATTTAAGAGAGTGTCAGTGGAG
ACCAGGGCGTGGCTTTGGTGGTTAAGTGCTTACTGTGCAGCTCTAAGACCCTGGGTTTGCATTCCCAGAGG
TCATGTGATACAGCCAAGCATGGTGGTATTCATCTGCAACCCTAGCACACAAGAAAATGGGAGAAGGCTTG
CATAATCTTTGCATAACTTTCATCATATTACATGTCCATATTAACATCACCAGGTTTATAGTATAGGTTTT
GGCTTCACTTGTAAAGTTAGGTGACAGCAAAGTTGAAGATATAGGGACATGGTGTAGCAGTAGAGTGCTTG
CTTAGTATGCTGAAAGCCTTAGGTTCAGTTCCAGCATTCTTCTACCCCTCTACCTCCCAATAAATGTAAAA
GTTGAGCCCTTGCTTTTAACAGTCAGAGTTAAGCAGGTCAAGGCAGGAGAAGCAGCTCAGTGATGGAGTGT
GCTTAGCATGCACACAGTCCCGGCCAGGACCACCAGCCTTGTAAGCAAAGTTAAGGAAACCCACTCAGACA
GACCTTTTTTAAAATTAGGGAGAAGGAAAATCAAATCATTTCTAGTCTCACCACCACTAATATTGGTAGTG
CTGGGGGGAGGTTTAAGACTAGCTCCTGACTTAGTCCTCATAACTTAGTGGTGCAGTAGAAGAAGAACACT
GAATCCTGGAAGGGAAGCAGGAAGCAGTGTAGGTGGAAGCATTGTCTCTGAGTTTCTATACAGATTAGTCT
GGGAAGAATACGAACTGTGGGTAAATGCTAACCCTAAGCCTCACGCTCGTTCTGCCAGACTTGCCTGCGTG
GCACTTGCCCTTCTGAAGGCCAATATACTACCAGGACTGACAACACAAACTGTGATTTGGTTAGTGATGAT
ACTGTATACATGTGTGAAATTTTCAAAGAGTTAAAAAGTTATCGCCCAGTGAGTGGTTGGCCATTTACACT
TAGAGTCAAATAACACTTTAGTAATACCAGCTGTATTAGTTTAGATTTCTGTAAATGGTATTTGCTAATCT
AGAGTTTAAAGTTTAGACAAATCTTTTAAAAGTTTTTTTTGAAGTTTTATTTTAAAAAAAGATTTAGTATT
TATTTTATGTATATGAGTACACTGTTGCTGTTTTTAGACACACCATAAGAGGGCATTGTGAGCCACCATGT
GGTTACTGGGAATTGAACTTAGGACCTCAGGAAGGGCAGCCAGCGCTCTTAACCATTGAGCCATCTCTCCA
GCCCCTAGGCACATCTTTTAAGAACACAACTTACACAGAGACAGGAAGGTGATAAGGAAGCCCAGCAAATG
ATAGCCACGCTAGACTGCAAAGACGAAACAGTGTGGTGGGCTAGAGAAACAGGTCAGTGGGTAAGAGTACT
GGCTGT
TCTTTCAGAGGATCCAGGCTCCATTCTTAACACCCACGTGGTGGCTAACAGCTGTCTGTAGTCCCAGTCT
CAGAACACACCTTCCTCTGGCCTCCATGGCACCAGGCATTCGATGTGGTACACAGATACACACAGGCCAA
ACACCCATGTGGCTCTCTGGGTCTTTACTGTTCCACTGCGCTTGCTTCTCTCGGGACCTTGTGAACTGTT
CACACCAAGTCTAGACGTAGCAGGGTGAGGATGGGCGAAGTCAGCAGCTTCCTGTTGCTCACAGAGACAG
AAACAGAGCTGTCTGCTTCCTTAGTAGGTTCCCAGTGACAGCTCCGGCTGCTTACTGCCGTAGTCTGCCC
AGGAATGGGCCGTTATGGCTCTTCCTTCCATCTCCTCACTATGCTTTCCAAGGTCACTCTCCTCATCCAC
CCAGAAGTCTACTCTCAGATTGCACTTCTAGGAAACCTCAAAATAAGACCAAATAATATAGTAAGACAGT
TTTAACCTTCCTTATAAATCTGTAAAAATTACCAGGGTTGTCAGTAGGAAATAATAAAGCCAGAGTCATG
CTTGAGAGTTTTAACACATTTTTCTCAGTAACTGATGATGGAGAGCATTCATAAAGATACAGATTTGAAC
AGCATGATCAGCAAACTTGACCCAGTTTATAACTATAGAACTCAACACAAGAGAACTGCAGAGCATGCAT
TTCTTCTGCAGCACAAACTGAATGATTTCCAAAAATGGCTCTATCCTGGTCCATGAACAAAGTTTTGAAA
TCATAGATAAACTCTCTTAACATAGCAGAAGTAAGTTAGAAAAAGATAACCTTGAAAAAAATCTGTGTAT
TTGGAAATTAAATAAGTTATGAATGACCCAGAGAAGTCATAATAGAAATGGAAAATAGTTTGAATAGTCC
CAGACCTCAACTCTGGAACTCTAATAATCCAACTGTAAGTAGCCATACAGCCTTAAAGTGTACCAAGCAG
CACAGGCAGGAGATAGGGAGAAATTATGTCCATGGAACCTATAGAAGTAACACAGCTCTCTTGCTGTTGA
TAGAACAAGCGCACGGAAAGGCCATTTAAGGTAGAGTGCTGATCAGAATCAACTTGACCTAGTTAATCTT
AAGTGTGTTCTTCTTAAGTGACTGGGACATTTTACTGCAGTCAGGCGGCTATCACCATAACCAGCAGATT
AAGTTGTATATTTTTATAGCAAAGTAGATTTAAGCTAAGATTCAACACAATAAAAGACAACTTTTCAAGT
ACTTGGAAATTAAGCAATATACTTAATGACTGTTCTGCTTTTAGGTTGGAATATTGCATCAGCAGATAGA
AGAACATGAGAAAACCAAGCAAGAAATGGCCATGGAGTACAAGGAGGAGTTACTGAAGCTGCAGGAGGAA
GTGAGCCTTTCATTGTGTGCCGTGCCGTGAGGTGTAGTCGTTCTCTTTCTGTAACTCCTGACTGTTGTGT
TGACAACTTGTTTCTGATTTCAGTGGTGAGAACTGCACAGCCTGTGTGATAGTCTGTGGGAAGAATGTGG
TAGAAATATTTTTCTCTGACCTTGAATCTAGGTAAAGAATCTTGTCCAAGCATGTTTGCTAGTACCTAGG
CCAGCAGTTAGCTGAGAGGAAAAGAATTGTTTATATAATATTGAAATCTTGATAACAGAACATTTAATTT
TTTTTTCCAAGACAGGATTTCTCTATAGAGCCCTGGCTGTCTTGGAACTCACTCTGTAGACCAGACTGGC
CTTGAACTCAGAAATCCGCCTGCCTTTGTCTCCCAAGTGCTGGGATTAAAGGCATGTGCCACCACTGCCT
GGCAACATTTAATATTTTAATTTCTAAGTTTATATTGGAAGATACAAAGCACTTAGAAATATTTTGGGAG
CCATGAGGTGCTGGTGCACGCCTTTAATCCCAGGACTTGAGAGGCAGAGGCAGATGGATCTCTGTGAATT
AGAGGCCAGCCTGCCTGGTGAGTTCTATAATTGCCAGGACTACACAAGAAACCCTGTCTCTAAAAACCAA
AAAGGAAGGAAGGAAGGAAGGAAGGAAGGAAGGAAGGAAGGAAGGAAGGAAGGAAGGAAGAAGAAGAAAG
GAAAGGGAAGGAAAGGAAAGGGGAAAGGAAAGGAGAGAAAAAAAAGAGCTGCAAAAAGCAGGGGTCGTAA
GATGGCTCATTGGGTAGAGGCACCTGCCACCCAACTTCATGCATGAATTTGATTCCAGGAACCTACATGG
TGGGAGGAGAGACCCAGCTTCAGCAGACTGTCCTCTGACCTCCTCTTGCTGGGCTGAGATGCACACACAC
CTCCTCTCCTCTCTCCCCTAATAAAAAATAAAAATGTTTTCAAAACACTTTTTTAAGCAAGAAAATTGGG
AGGCAAAGCTAGGTGAGAGTTGAGAGTGTACTGTTCGTGCAGAGGGCTCTGGGTCAGTTCCCAGTACCAT
GTTAGGAAGCTCAGAACACCTAGAATTCCAGCTAGAGGGCTCGGACATCCTCTCTGGCCTTCTTAGGCAT
CTATCTGCACTTGCATGCATATACCTACATAGAGAGAAACACACATAACTAGAAATTAAAAATAAAATCT
TTAAAAAGAAAATTCTATTTTCATATTCAATGTAATTTTTAATATTGGGGAAATTACTTTTGTTTTGGCT
TAAGTCAAAGTCTCCTACTGACCGTGCATCATCAGTGCCCTCAGGAGTATGTCTTGAGAACCACTGGCTC
AGAGGGGAACAGTCACTGCTTCCTCTATTCTGTGGAGTGGGTCGTGGAGGGGCATCTATGAATGCATGAC
AATTGCCATTAAACTAAATTTTACTTTTTCTTTTCTGGACTGGAAATTTCATTACTTTTATAAATTTCAT
TTCTTTCTCTCACATACACATGCAAGCAGGCACATGCACTTTAAGACAAGGCAGCCCAGGCTGGCCTCAG
GTTCTTCATGTGATTGAAGATGACCTTGCCCTTCTGTTTCTCCTGCCTTCACTTAGTTCTGTGATCACTG
CACCAACTCCCACAGCCAGTCTGTGTGGAGCTTGGTCCAGCCCAGGGCCAGCGTTTTCCAGGTCAGCGCT
CTACCAGCTGAGCTGTAGCCCAGCCTTGTCGTTTTTATGTCTTCTTTTATTTTGCTTTGCAGCTCTAACA
TGCTGCCAATCTATTTTCAATAGTGTAATTTAGCAGTGCTCTTGCTGTATAATCCCAATTCATCTGACCA
CTACAGTTTGCTAACTCTTAAATAACTGTTTTTACAGTTAAGCAGACTGAAGAGAAGCTATGAAAAGCTG
CAGAAAAAGCAACTAAGAGAATTTAGAGGAAATACCAAAAGTTTTCGAGAGGATCGGTCTGAGATTGAGA
GGCTAACTGGAAAAATAGAGGTATGTCTACAGCAATAGTCCCAGTTGAAACGGCAGTTGTCCATTGTGTA
GGAAAATGCCATTTCTAATGGAGCTGTTTTCTGGAGCAAGTTCCTGCTATCTCAGGGGCTGCCACTGAAC
TAGTCAGTGTAGGATGGCGGTCCCCATCACATGCTCCCCTGTGGCTGATGGGCTTCCAAGACATCAATTA
ATCTCAAGAGTGTAGAGCTCCCATACAGAGCACTTGGAAATATATGGAGGTTTTGTTTTGTTTGTTTTTT
GTTATTATTTTAAAATGATGGAGCATGAAATTGGCATTCATGGTAGAGGTAGCAGGGATAGCCAACATGT
CTGGGACAGTATCCTGGAGAAGATCCTAGTACTCAGTACCTCCCATTCTGAGAAACACGGAGCATCAGTT
AGTGGCAGCATCCTACCTTATCTCTGTCTCCTCCACCCCGATCCTGTAGGTGTTAGTGACCGGGGGGATG
CTTGGCTTTTCTTGTAGAGAGTGCTCTGCTCATCAGGTGACTTCCTTTCAGTTCCCATTTAGGTAGAGCA
TGAGTTACAATGGATTCGCCTACCTCTCTGATTTTGTTTTTGTTCCATAACTCTGTGATTACTGATAGGA
GACAGTGAGTCCCCTCTCTTCTCCAGTACATTGAAACAGTCACTCAGGATTGTGGGAACTAAAAGGGGGA
GGGGAAAGAGGGGGAAGGAGGATAGCCTGCGCTGGCCAGAGTTCCTCCTATGCTCTGGGCAGGCGGATGT
GGGAGGGCTGCCAGACGCTTTCCATTCGCTTTCCACTCGCTTTCCAGGTGGGCATCTAAGCCACTGACCC
CACTCGACGGGGGCGGGGGGGGGCGGGGGGTGGACAAGGGGCAGCCCCCGAAACCAGGGGGCCCAGAGCA
ACACCCTGCAGCCCTGGGGTTATGGGAGAGAAGGCTTAGGGGAGAGGTTCCCACACAGGCGAGAGTCTGC
ACAGTGGGCCTTGAAGAACAGACAGTTTATTTAGAGCTTTATTATAGAAAGGCAGGGGGAAGAGAGGAGG
TAAAAGAGAGAGAGAGACTGGCCATGGCCAAGAGGAGAGAGAGAGAGAGAGAGAGAGAGAGAGAGGAGAG
AGAGAGAGAGAGAGAGAGAGAGAGAGAGGAGAGAGAGAAAGAGAGAAGAAGAAGGAGGAGGAGGAGAGAG
AGAGAAGAGTAAGAGCAAGAGAGCAAGAGAGGAGTGAGAGAGTGAGGAGGGGCTAAGCAGCCCTTTTTAT
GGTATGCTGCTATCTTTACTGTTGCTAGGTAACTGGGGAGGAGTTTAGCCTGAAGGTCAGAAGCTTGGGA
CATTGCCTACATGACTACTAGCCACGCTTCTCTTATGGGGGGCTGAGGGGGTGGGAGCCAGGGTCCCAGG
AGACATGAGAGAACTCCTTCTGTCCCATGTAGGTGAATTATCACCACCAGGACCCGGGGTTCAACATCTA
GCTTCAACTGGAAACCAGGCTGTCTGTGCATAGCCCAGTGCCCCACACAGGATGTTTTGGGACTTCAGAG
TCTGATCCAGTAGTAGGCATGGGACCTAGGAATCTATACCTTACCTAATTAAGAAACTGTTCAATATCAT
TGTGTGACTGCTTAACAATGTTGTATGAGTGATCTGTCATATAGAAATGTACTTTATATACATGTCTTAT
TTAGGGTTTCATTGCTGTGAAGAGACACCCTGACCAAGGCAACTCTTCTAAAGGAGAACATTTATTGGGG
TTGGCTTATTCATTCAGAGCTTTAGTCCATTATCATCATGGTGGAAAGCATGGCAGCATGTAGGCAGACA
TGGTACTGCAGGAGCCGAGTTCTACATCTTGATCTGCAGGAAGCAGGACATTGGACTTCTACACTGGATG
AAGCTTGAGCATAGGAGACTTCAAAGCCTATCCCCACAGTGACACACTTCTTCCAGCAAGGCCACACCTA
CTCCAACAAGGTCCTATCTCCCAATAGTGCTACTCTACATGGACCAAGCATTCAAACACATGAGTCTCTG
GGGGCCAAACCTATTCAAACAACCACAATACTTTACTTTATGGAATTGTAATTTCTATGTTTAATATTCC
TTTTATATTTCAATATTTTTATAGATTTAGAACTTAGAATCAGAGGACCACACAGATACTGCCTCTCCTC
CAGCCTCTCTACTGCTACCATTTCTTCTAGCTCCCGCACATTCTTTTTTTTTTTTTTTTTTTTTTCAAGA
CAGGGTTTCTCTGTGTAGCCCTGGCTGTCCTGGAACTCACTTTGTAGGCCAGGCTGGCCTCGAACTCAGA
AATCCGCCTGCCTCTGCCTCCCAAGTGCTGGGATTAAAGGCGTGCGCCGCCACCACCCGGCGCTCCCACA
CATTCTTATCACCAGCCTACACCACCAACCATGAGCACAGAGCCTCGGTAGCCATCCTCTGGTCCAGGTT
ACTTCACTGACTCTGGATTCAGATCGTTGTTATCTTTAAAATCCTTGTCATCATAACCTTTGAGAGTTTC
ATCCACAGACATAGTGCTGATCACGCTCTGGCCTTGGAGTTTCCTGGAGTCCTTTCTTCGTAGCTATTTT
GATCTCCTTGTGTCATCCACTTACTCCTGTTGACATTCCTCAGTTCTTACCTTTATGAAATTGCTTTCAG
TAAGATAGTCAACCCATGAGCATGACAAGCCTTGCCGTCCTTGGTGTCTTCTTTCATTTCTTTCTTTAAT
GTTTTAAAGTTTTCATTGAAGAGATTACTTCCCTAGTTATGTTATTTCACACGTGTGTGTGTGTTGTGTA
TATATGTATGTATGTGTATAAATATATGTGTGTGTGTGTGTGTTTATTTAATTCTGCAGTATTTTAAAGT
TGTTATGAATGAGATTGCTCTCCTGAGTTCTTTCTCAGGGTGTCTGTTATTGGTTATGGAAAAGCTACTG
AGTTTTGATGTGGGTTTTATGTCTTGCTCCTTTTCCGAAGGTGTTTATCAGTTCTAGAAGCTGTTGAGTG
ATGGTATTCAGAGTCTCATGTATGAACCATTTTGTGTAAAGAAAGGATCATTTAGCTTCTTTAGCTGTAT
CCCTTTTCTTTCCTTTTTGGCTTTCTGTTCTAAGACTCCAAACTTGAGTAACAGTGGAAAAAGTGTTTGC
CTTTGTCTGTCATTCCTCCTTTTAGTGGCAGTGCTCTGGGCTTCTCCCCATTTAGTGTGTTGATTATAGG
CTTGCCATATATGTCCTTAATTATGTTGAGCTGTACTTCTTTGTGTTTGTTTGGCTTTTTGAGACAGGGT
TTCTCTGTATAGTCCTGCCTATCCTGGAAGGTACTCTGTAGACCAGGCTGGCCTTGAACTCAGAGATCTG
CTCGCCTCTGCCTCCTAAGTGCTGGGATTAAAGATGAGCACCACTGCCACCTGGCTGACATATAGCCTTT
TTAAAATTGATTCTTTGTGAATTTCACATCTGCACCCCAATCCCACTCATCTCCCCATCCCTTCCTCCCC
CCCCCCAGTGCCAAGAGGAAACAAAAATAAAACCAACAAAACAAAAAACCCCAAACCTCTCCCTGTGGGA
GAGGCAGTGTGCCATATAGTATCCCCTTTTGTCCAAACAGCTACACTTGCAAATGTTCATTGGAGTGAGT
CATTGGTCCAGTTTGAGCCCCCTGGCTTATGCTACACTATCAATACTGGATTGTCAATTGGACTTGTCTC
AGATATCCTGTTGTTACTCTGTGTCATGGAGATGTCCAGCCTTGAATCTGTAGGACCAGGCCCCTTCATG
GGTTACAGCAGTTCACAGATGGGATAGATGTTGGGGTTGGCCAACTCAGAGCCTTGGATCTGGGCCTGGT
ATATGGTCAGCCTGCCAGCTTTCCTGTGCCCGCACCACCAGGGCCAGCGCTCCTACTTTACTCAGGTGAT
TTGGCAGCTGGCAAGGGGTGGGATTGGCTGTCCTGCACTCATGCCATTGGAATCTACACCCGTGCCACCA
GGGCCAGCTCTCCAGCATTGTCCTGTGAGGGGTAGGTCCAGCTTACTCTAGTGTGTAGCTGGTGATGGGT
GGAGATATGAACTAACTTTATTGTTAGTTTTTTCAAGGCTATTGGATTATATCAGAAGTCTTTTCCGCAT
TAACTAAGAGGATCATGTGATTTCTGTTCTATTAATATGATAAATGATATTTGCTAATATATAGTATATA
TCATAATTTGATAACATTAATATGTATGTTGAACCATCCTTGTATCATTGTAATGTTACGTGTTTGGTCA
TAGTGGATGATCTGTTCTTTCCCTCTAAGGCAATATTTGTCTTTACCTGTAAGGTATGTTTCTTGCAAGT
AACAAATAGATTGATCCTTTGTTTGTTTGTTTTGTTAAGATTTATTTATTTATTTTATATATGTGAGTAC
ACTGTTACTCTCTTCAGACATACCAGATGAGGGCATCCAACTCCATTACAGATGTGGAATAACACTGACT
GCTCTTCTAGCACATCTGGAAGAGCAGTCAGTGTTATTAACCACTGAGCCATCTCTCCAGCCCCTGATTC
TTCTGTCACTACCTCCTCAGCACTGGAATTTCAGATGTCTGCTGCCATGCCTAGAATTCTCTAGTGAGCT
GGCCTATATGCTTACATGCCAAGCATTGTTCACATACCGTGTCATCTTAGTAGCTCTTAGATTGGGCCTT
TTAACAATGTCCTGTGGATCTTGTCATTTCCCTTCATATTTTCTTATTACCTTTGTCATTGTCTTCATGT
TCTAATTCATATACCTTGTCTTTAGGCCCTGAAACTCTGCTTTCTATTTGGAGAGGCTTTCCATTGAGCT
TTTAGTTGACTCAATGAGATTTTTATTTACAAAATTTCCATTTGATTTTTTTCAGTATTTCTGTCTCTAT
TGAATGCCTCATTCATAGCCTGTATTGACTTCTTACTTCATGCAGATGCTTGTTGGCTTCCTTGTTGAAT
TCCTTCAAAAGTCTGTTTATGTTGTTTGATTTCATTGAACGTTCTTACTCATTCATTCATTCATTCATTC
ATTCATTCATTCAGTGTGCATGTCATGTCTATCACCTCCACTTGAATGGGGCTGGGTTTCAGTTTATTCT
TATTAAATGCTATTCCTGAGTCAGTGCTGGCACATGTCATATAGACAGACAGACTGTGAGTGAAGGGACT
ATCAGAAAGCTGACTTCTCACTTAGCTAAGGAGCTCTGGAGCTCGAGATCCCTGGCGGACAAAGATGGGG
AGGCTGAGGATCTTTCCCTTTGAGTTGCTTTATTTTCAGAAAAGTTGGGCCCATTTTCATGACGTAGAGT
TTGAAATTGCTGTTGATTACCTATTATTTTTGAATTATTTGACTCTGGATTATAAGTACCTGATAAAATG
TGATGCATATTTTCCATATTATTCAAGCTTTTGCAATTCTACGAAATAAAAAATTTTGACTATCAAAGAT
ACTATTATTGGCAGGATAGGAAGTGAGAATCAGGCTGCTGAGTTACTGGTTTAGAAATCTGTTCCAATCT
ATTCAGTTTCTGGGTCTGAATGTTAGAAATTTAGATGGCAAAGGGAATATTATTATTTTAGCGTCACTTT
TCTAAAAATACAAAATGGTAACAGCCCATAGGTGGCAGCAGATCTGCAATAATGCTCTTGAGCCCACTGT
CATGGCCGTTTCTTCCTCTTCACCACTAAGTGTTACATTTAGAAAGACAGACATGTAGAGGCACAGTGTG
AGCAGGATCCAGTCCTCCTTTGCAGCGTGAGTAATGGGGCTCCTGCTGAGAGAAGGGCGCCCAGGGAGCA
GGGCGCTGTGCAAGCCTCACAGCCTGGGAGCACTTAGATGTTAAGTACTTCACTTGCCTCCCCAGGAATT
CCGACAGAAGTCTCTGGACTGGGAGAAGCAGCGTCTGATTTATCAGCAACAGGTATCTTCTCTGGAAGCA
CAGAGGAAGGCTCTGGCTGAACAGTCGGAGATAATCCAGGTAGGCTTAAGCTGTCCGATGCATGGCTATT
GACCGAATCGGACTTACTGCCATGCTCTTTCTGTCTTACTTGGATTCTCTCTGTAGTCTTTGTGTGGTAA
GCCTGCCTTCCTCTACTTGTGGCTTAGCCTCTTCAGCATCTAAAACACTCTAAAACACTTCAGTTTTTTC
TTTCTTTTTTCTCTCTTTCTTTCTCTCTTTTCTTTCTCTCTCTCTCTCTCTCTTTCTTTCTTTCTTTCCT
TTTGGTTTTTCGAGACAGGGTTTCTCTGTGTAGCCCTGACTGTCCTGGAACTCACTTTGTAGACCAGGCT
GGCCTCCAACTCAGAAATCCGCCTGCCTCTGCCTCCCAAGTGCTGGGATTAAAGGCGTGCACCACCACTG
CCCGGTGAGTTTTTGTTTATTCAATCCTCATTTTATTCATGTTTATTGACTATTATCTTTAAGTCACTGC
TATCAATTTACTATTGTGGTTAATTCCATATAATGCAAACTTCACTGTTAGTAAAAGTGGAGTTGTTATT
TACCAGGCAGTCAACTAGAGATAGAATAAGATACCTTGAAAGAACTGACATCTGTTGTGCCCACTAAGGG
TATTTGAATAATCCTTCTCATAGTTTTATTTAACATAAAGTTATCTGATTAGTTAAAAGTATTAGGTATA
AATGTCTTTCTCTTTTTCCTTGGCAAACTAACCAAGAATGAAAGCTTCCTTTAACTCAGTTTGTGTCCAT
CAATGCTGTGTTGTTATCTTTACTAAACACTTATTAAATCTCTGTGTGCCCATTACTTAGTGTTAATGAT
TGTCATCATCTTGCCAATCTTGTATGTAAATGAGCATTAGAAATTCATAACAGAAATTATACCTCATCTT
CTTTTCGTGTGGTATTGAACCCATGGCTTCACTTATTCTTGGCGAATACTGTACCAGTGAGCTAAATCTT
GTGCCACCCTGACTGGCTTTCTGAGCGCAGTAGTGTTGACAACATGTGCACATTGTTTTGTGCAGCTGTT
CTTTGAGACTTCTTCCTTTTTTGTTTTAAAGAGGGACATGGCATTAAAAAAATAAAAGATTGATTTTCTT
TGTATTGGTATTTTTCCTTGATGTATATATGTGACCACTGTTACACAGTGCCACAGTGCCCGAACAGGGC
ATTGATTCTGGACTGGAGGCATAGATCAGTGTGAGGTACAGGGTGAATGCTGGGAATTGAACTCTGGAAG
AGTAGCCTATGGCGTTCTTATCCAGCCCACTTCTTTCATCTTGGAAAGCTAAAACTGGGTACACTTTTAC
TCTTTATTTTTTGCTATTAAAATGTTACAAAGCAAATCATGGCATCAACTCATTTTTGAACAGGCAAGGA
CTTAAAATCCCTATAATATGATAGGGTAACATCAATAGGATAAATAATAGTAATATAACTTAGTATTCAG
TCTGCAGTTTTATCCAGTTGTCTAAAAACTACTCTTTCACAGTGGCTGTTTGAGGCAGAATCCAGTTAAG
CTTGACTAATTGCATTTAATTGATATGTGGGACAGCAGAAAATCCGAGAGTCTGGGTAAATGTTATGAGA
AAAAACTGAAAAAAACTCTTTCATATTTTTAACATTGAAGAAGATTCAAAAACAGCTTCACGCACTTTGA
GTCATGTATTTGGATTCATCTATGCAGCTATTAGAGTTTTATACTCCATGCTTGTTTTAGGGAAAAAAAA
AAAAACAAAAAAACCCGAGAACATCCTTTGTCTGGCTTTTCTTTTTCTTTTTTCTTTTCTTAATGTAGTT
TTGGAGCTTCAAGGAAACCATTGTCACAAATAGATTGCTTAAGAACAGTGGGGTTGGAGGCAGCAAAGGC
TTTGATTTTCCACTGCAGCACGAGTACCTGTCTGCCCATCTGGGTCGCCTTCAGATGCGGCCACGCAGGC
CATAGCATCCCTGTAGGCAGCAGCCTGAGGATCCCACAAGGCTCTGAGTTCAGTGTGCACCATGCCTCTG
CGGTGTCTGTCACCTGGTGCGCCTTGACAGAAGTGCCTTCTGTCTCCCTAAGGCAAATTGCCTTAATGGT
TTCCTTACTTTCATTGCCCTTTGTGTAGTGAGATAACTCCACCCTTCTGAATGCTTAGTGAAGAAGGGCA
TTTCTGCTTTCTTGAAAGAAAATGTATTTGTGCATGAAAATATATATTTCAAATATATTTACCTTTAAGT
GCTTCTAAAGCTGGCCAGTTAAGAGGAGGAATTTTAGGAGTTGTTTCTGTGATTTTCTGACTTGCTTAGG
ATTAAAAGAGGTATTTCTGAAATTAGTACCACTGTAGCAATATGTGAATTACACAGTTTACTATTTTGTG
CAGTTAGATTAAAAAGGTGAAAAGAAATATAAGATTAATTTAATATATTAATATATAAAAATTAATATAT
TATAAATTAGTATATATTATATAAATATATAAATCAATATATTTAATTTAATAATATATTTTATTTAACC
CAGTATATCAAAATATTATCAGTTCAAAATTTACATTCTTTTAAAAACTAAGTCATTGACACCTCACTGT
GTATTACACAGTTAGAACATTGTTCAAGTCAGACTAGCCACAGCTCAGTGCTCAGTCTCTTATTGCCATA
GTAGACAGTAGAACTTGGGCTATCATTTCTTTTTTTTTTTTTTAATTTTTTTATTAGGTATTTTCCTCAT
TTACATTTCCAATGCTATCCCAAAAGTCCCCCATACCACCCCCCCCCCAACTCCCCTACCCACCCACTCC
CCCTTTTTGGCCCTGGCCTTCCCCTGTACTGGGGCATATAAAGTTTGCAAGTCCAATGGGTCTCTCGGGC
TGGAGAGATGGCTCAGCCGTTAAAAGGCTAGGCTCACAACCAATGGGTCTCTCTTTCCAGTGATGGCCGA
CTAGGCCATCTTTTGATACATATGCAGCTAGAGACACAAGCTCCGGGGTACTGGTTAGTTCATATTGTTG
TTCCACCTATAGGGTTGCAGTTCCCTTTAGCTCCTTGGGTACTTTCTCTAGTTCCTCCATTGGGGGCCCT
GTGATCCATCCAATAGCTGACTGTGAGCATCCACTTCTGTGTTTGCTAGGCCCCGGCCTAGTCTCACAAA
AGACAGCTACATCTGGGTCCTTTCGATAAACTCTTGCTAGTGTCTGCAATGGTGGGCTATCATTTCTAGT
TAATGAAGATACTGTGTTTTTGCATGAGTGAAATAGTAGAATAAAAGAGGACCGAGACTGATTCTAAAGT
AACAAATGGAGACTACCTGGTTACAGAGTTGCTGACACAGCTAAAGCTCCAGCATTGAATGCAGACCACA
GTGTTATACAGTGTCACAGTGCTGAGGTTTTGGGCTATGTGCCTCCTGGCCACTCATTTGGACAGCTTTT
CCCAGCTGTGCTCTGTGAGCTCCAGGGCTGGTGGGCAGGTGAGGGATGGGGTGAGCTGAGGCTCAGCAAC
GGAGCACAGCCATTTTCTCCTCCTACCTTCAACCAATCTCTACATTTACTTATTGTGCAGAGGATTCTGT
ATAAGATTTCCTCTTGTTTTTGAAAGTCTTCTGCCAATTGCATGCATAGTGGTTAGCGTTGACCCTGTCC
TGAATCTTCTTTCACGTGCCATTCTAGGGATCTGGTGGAAAACAAATTGCCTCTACTCTGAGGCCTTGAG
CTCTAATTTACACAAAATTATTTGTCTGTATCTTTGCCTCTCCCTTTTTGTTTCCGTTTTTTTTTTTTTT
TTTTTTTTTTTCATCTTTGTGTCACCATATTGTAAGGCAAAAGAGTTTGGTCCTCAGTTATGTACAGCAG
AATCTTTAAATCTGTAGTAGATGGATTCCACAGAGATATTTCTGCTTGAATTTCTAAAATACAAGTGGGT
GCCTTTCCTTTAGGAAATGTTTAAAATAAGGGAAGTTTAACTTTACAAATGATTTATGTCCATTTTTAAT
GTATAGATAAAAAACCAGTCTGGATACTGAATTAAAACTTGAAAAAAACAGAATGTGAGAAGCCAAAAAC
ACTTCAGCTGGCATGCAGAGCCAAAAGGTTAGACACTGTACTCCACGGGTCCTGAGCCAGTTGCTGAAGT
GGCTTTATTTGAATAGACTTCAGTAGGATATGCCACTAAAAGAAAATGTCCAAAACCTGCTGATAGCCCA
GCCTTCCTAGGAGTGAATCTTTTCTTTCCTCAGTGAATACAATTATTGATTATTCTTCTATCAACTCTCT
TTTCCCTATATGTTTTTAGGCTCAGCTTGCCAACCGGAAACAGAAATTAGAGTCCGTGGAACTATCTAGC
CAGTCAGAAATTCAACACCTGAACAGTAAGCTTGAGCGGGCTAAGGACACCATCTGTGCCAATGAGCTGG
AAATAGAGCGCCTTAACATAAGGGTCAACGACCTGATGGGAACCAATATGACTATCCTGCAGGACCATCG
GCAGAAGGAGGAGAAACTAAGGGAATCTGAGAAGTTACTAGAGGTGTGTGTTAGAATGAGTAACTTAGGG
AGGTATATGCATGCCCTAGGACTAGGTCCTGGATGCCCAGCTTCCTTCTTGGGCTGAGGCTGGTGGCTCA
GCTCCTGATAGCTGTATTCATACTGTCCTTGTCATAAAACTTGCAGTCAGCCTTCCTGGTTGCTTTTCTC
ATGAAGTAGAAGCATTGAAATATTATTTGACTTTTTTTCTGGAGTATTTTGCAGTCTCTGCAGGAAAAAG
AGAGAGCTAAAGGAAAGTTGTAATAATATCTGTCTTTTTTCTTGAGTGTTTTGCAGGCTCTGCAGGAAGA
ACAGAAAGAGTTGAAGGCAAGTCTTCAATCTCAAGAAACCTTCATCCTTGAGGCAAAAATGCAGGAGAAA
CTGCAAACGACATTAAAGGCAGTTGGCACTCAGCAGTCCGTAGAAAGGTCTGTTTGAACCGTAAATGTTA
GTCTGCTGGCCTGTTTGACAGTTTGGCATTGAATGCTGATTTTAATATCGAATATTTTCATTTCTGTGTT
TGATCATCTTGTCTTATTTGACTTTAATTTATGAGTTAGATTTTTATTGTCTTAAAATTTGTATGGATGG
TTTTGTCTCCTTTTCATTTAAGTCTCTAAGATTCTGAGGATCATAAGGTTGGAAACTCCTTATCTCTCTT
TGTTAGACTTGGAGGGCCTCTCTGCCTTAGAAGACTAAGGTATGGTGGAAAGTAGTTGCTTGCCATCAGT
AACAGCCTGTCACTTAGCTAGCCCTTTGGGAACTGACTTTATTGTTATCATTATTATTGTATTATTATTA
CTACTACTACTATGTTTTGAAATAGGATTTTATGCATTACCCCAGGCTACCTTCAAAATTACTGTGCAGT
TGAGGATGACCTTGAACTCCTCCTGATCCTCCTACCTTTGCCTCCTAAGTGCTGGGATTATCAGTGTTGT
GACCCCACCCAGTCTCAGGGCTTCCTGTATGCCCTGGGAATTCTCTACCAACTAAGCTATAAATCTCCCG
CCCTGGGCTATTGCTAGGGCAGGGTTATAGTAAAAAATAATAGAGTAAATGAGCTTCCTTTTGTGTAAGT
CAGGCATGCTTCTGTTCTTGCCAGACTGTCTTGTAAAGTTTTTCTGATAGAAGGATTCAAGTGTTAACAA
AATGGTTTTGCTGTTTTATGCGGCTTTTCCCCTTGAAACACACCCCAAACCCTAGTAAAGATTAAATTGC
TGCTGACCCTGGATTGTTCTCCAGGTATTCCCCCTCCTCCCCAAAGCAGGCTACAAACTGACCAGTTCTA
AGGAGCTTAGTTAAAAGAATAGTTGGGACAACAGCCTGGCTGGCATTGTTTGTTTGCCAGGATAAGAGAG
AGACCTGTTTCCCCCTCCTTCTGTTCTTAGGCCTCTGGAGGACTGTCAGAAGGAAAGGAAGTACAGCTCT
CCAGGGCAAGGAGTCTTGGATAATGTGCTCTCCCAGCTGGACTTCAGCCACAGCAGTGAAGAACTCCTGC
AGGCAGAGGTGACGCGTCTTGAAGGCAGGTAGATAATTAGCTACACATTGAAAATTTGAAGTTGTTAGGA
TGAACTTTGATTCTCACATTGATTGAGTAAAACAGTGCATTGGTGAAATGAAGTTGTTTTTTCTCTAGCA
TTCACAAATGGCCTCACTCCCTAGTTCATTGAAAATACAGAAGACACTGTCCTAACCTTCTGCCAGTTGG
CCTTCATCTGAGCCTGTGCTCTGTGGACGTGTCTGAGGCTGACTTGCTCCTGCTGTGACGTCCTCTTTAG
GGCTTTCTTCAGAATGGTTTATATGGACAGAAAGTATTCTGGGAGCTTTTTCCATTTTGAAAACAATTAA
ACCCCTTTAAACCATCAGAATATTTCCTCTTTCTCTGTTCTTCAGAGTCAAAATCTTTTTTTAAAAAAAT
TATTTATTTATCTTTTTTTATATGAATACACTGTAGCTGTCTTCAGACACACACCAGAAGTGTGTGCATT
GAATCGGATCCCATTAGAGGTGGTTGTGAGCCACCATGTGGTTGCTGGGAATCGTACTGCTGACCTTTGG
AAGAGCAGACCGTGCTCTTAACCACTGAGCCATCTCACCAGCCCTAGAATCAAATTTTTGAAGACTTGTG
TATTCTGATAATTCCCACTTCACCCTTTGCCTTTTAGGCCCCATTCAGTTCAGCTTTCTTCTGTATTCCC
TCACTGAAACTGTTCTTGGAGAAGATACAGTGGGCATGTCTTTCTTCCTTCATTCTTGTTTATGGTGTGA
TTTAGATTTTCTTGGCTTTGAATTGAATGCTAGCTAATGGCAAATCTATAGTATTTGGTAGCATAGTATA
TTCTAGCATAGCATGCATAGCATGGCATAGTGTGATAGGTAGTGCTTTTTGTTTTATTTTATTTTTTGCC
ATAGTTACAAACTTTATTTTGCTTGTCACATTTGTAAACTTTTTTATTGGGAAGAGTTTGATATTTTGTT
TTAAACTCATTATAATTGCTTGTATACTAATTTTTTTGGTGTGGGTAAAAGATAAAATATTTACATAAGC
TCTTTGCCTTTAGGCAAACATAACAAGAAGAAGTGCCACTTTACACTTTCTAGCTTGTCTTGATACATTG
CTCCTCAGTGTGCTGTGTTTCGTGCAGCCCTCTGCTGTAGGCCTTTTAGAGTGTGTGGGGCACTTGTGAT
CTCTACTGAGGTGTGTGTGTATATGTGCTTTATTTAGACTGAGCCAAACAAAACTTACTATTATTACTAC
TATTACTAGTATTGATATCATTTGTTTTTTTGAGACAGTTTTTTTTCTGTGTAGCCTTAGCTGTCCTAGA
ACTGCTTTATGTAGACCATGGTGGTCTCGAACTTAGAGATTGGCCTGCCTTTGCCTCCCAAATGCTGGGA
TTAAAGGTGTACTCCACCCACCATGAGTGCTAAATAAAGATTATTAGTCTTTTGATATTGGAACAAAAAA
ATTTGTTTTTAATCATAGTTGTCATCTCCCCCTCCCCCGACCTCCAGTTGGTTTTCTGATAATCTTCATG
GACTTCTTGTCAGGCTCAGAAGTTGCGCCTCTGGTTTGACTCCAGCACAGCACTGCACAGCACTGCACTG
ACCACCTTAGCCCCTCAGGTCCTGCTTTTCCCATGCATGGCTAGGACAATGTGATCTCTGATCTTACTCC
TCTGCTAGTCTCAGTGTTTAGCACATTGTAACCATATACTAGTAGTAAATAGATAAATAAAAATTCTAGA
ATTTCTGGAAATAAAATTAGAATGAATAAATAACAGTATTCAGTAAGAATGGCTTCAGTTTTTTTCTTTG
AAAAATCTAGTGGACAAAAAGACTGTTCTAAACAGTGTGGTGTTTAGTTTGAACAGCACCTAAACCAGGAA
GGGCAGGGCTTTTGTTATACTACAAAGATGATATCTCTATTTTATTTTATTTTTGTTTCATGTGCATTGGT
GTTTTGCCTCCATGTATATCTGTGTGAGGGTATCAGAAACCCTGGAACTGGAATTATAGACAGTTATGAGC
TGCCATGTGGGTGCTGGGAATTGAAGCTGGGTCCTCTGGAGGAGAAACCAGTGCTCTTCACTGCTGAGCCA
TCTCTTCAGCTCCCAAAGATGATATTTTTGAATAATAACTTATCTGGCAAATTTGCTATGTTTGTGAAATA
ATATAGTACTAAGCATTTGTAGAATAATTTAGAGGTGTATGCTGTAGCACGTGAGGGCTAATGGCTAAGTT
TGGCAGGCATAAGAAGTAAAATATTTTAACCATAGAGTTTTCAGTTTCAGAGATAAAATACTCATTTTAAA
GGACTTGGGGAATTAAATGAGCATATTTAAAGTAGATAATTTCTAGAAAAATTTGCCTTCAAGTTGTAAAA
ATTGAATTATTTAAGGTGAACAGGGGAGTAAGGACTGGAGTGACTGTAATTAGGCCAGTGCCAGTTTGGTT
CCTGAGAAGCTAATGGCCATCATGGGCTAGGGGAGGAGGTGCTTTGTGGTGAAGGGACGATTGCATTGCTA
CAAAGAGGCAGAGCACTGGAGTGAGAGGCATGCTGCCTCCGTGGCGCTAAGTCTCAGAAAGATTGGTCTTG
AGTAAGAACAATGAGTTGGGGACTCTTCAAAGAGTTCTTGCGGTTCTGTATAGTGTGGTTCTGTATAGTGT
GGAGTGTGTCTGTGTGTTGCTAAGGGTGATGAAGTGTGAGTCTGTTGTGACAGTTCTTGGTGGCTTTCCCT
CTCTCATAGAGCTAGAGAAACCCATACAGCTAGAGCAAAACTGTTAGTAAAGTGGCTGGTGTCTTTATAAT
TCTGTAGCCAGTAAAGTTTAAATCAGGGGGCGGGAGTGGAGGAAAACACGAAAGAC
AAAACTACCTCAGTGTTGAGATGGGTTTGCTGCATCTAGTGATAGGGGAATTGCAAATGACTTCTCGTGA
CGGGTATGAGGTTTAAGGTGTTAGTAGGAGGTATCTTGGGCATCAAGAGGGTACACTTAGATAATCCATC
TTATGGGTTTCAGGAATTTTACTTATATATGGTTGTATTTCTGCCATACAGTTTAGAATCTGTGAGTGCA
ACATGCAAACAGCTGAGCCAAGAACTAATGGAAAAATACGAAGAGCTGAAGAGGATGGAAGGGCATAACA
ATGAGTACCGGACAGAGATAAAGAAGGTGAGAGCCTCCGTCAGAGCCTGGGCTCAGAGCTGGGCAGGAGG
TGCTCAGTGAGGGAAATGTGTTGGGTGGTCAGTGGAAGAGTTTGCTGGAACTCAGTGGGAGGAGGAGGGA
TATGAGAGGATGATAGGGATGGATGATCAAAGGATACTGTGCATGTGGGGAACTTGTGAAATATGCTCAT
GATGATTTCTAAACAATTATATTGCTTATTAAAAAAGACATTATATTACCTGTAATTTCCTTATTGGCTT
CCTTTGTGCTATGAAGTAGTTAATGTCCTCATTGGCTGTTGGTACTCTAGAGCCTTTTTTTAAGGGGTCC
TGTCCCCAGTGTGCTGGTGTACTCTAATCCTAGCTTTGGGAGGTGTAGACAGGAGATCAGAGCCAGCCTG
GGTTAGATGAGACTGTCTCAAAATACCAAAAATAAATTAATTTTTTTAGAGGCTTTATTTAGAGGATTAG
TCTTGTTTTATAGCCCAGGTTGGCCTTAAACTCATAATCCTCTGGTCTTTTGAATTCTAAATATTGAGAT
TAGAGACATCTGTTACCATACTTGGCTTAATTTGTGTTTTTCACTACTTTGAGATTATTGCTAGTTCCTG
TGCATGTGTGTGTATGGTGCACACATGTATGTGTATGTAGAGCCCAGAAGTAGATATTAAGTAGCTGCTT
ATTGCTCTCTCCCCACACACTCACCTGCCTCAGCAGCTCTAACTGAACTCAGTATTCATTGGCTGACTAG
CTTAGCTGGCCATTGAACTGCAGAATCAACCTTTATCTGCTGTCCCTCTCCCCATGCTGGGATTATAGAT
GTATGCAACCTTCCAGGATGAATTTTATGTGGGGGCTAGGAATCTAGACTAAGTCCTTCTGCTCCTGCTG
CAGGTATGTTATAGACTGAGCTGTCTTCCTCAAAGGCTCTTGCCCCTTTCTACCTCTTATCTTTCTGTTA
GTTCTCTCCTGAGAAACAGAAAACTAGATAAAGAAACAAACAAAAACCCCAAACAAACAAACAAATTTTA
ATTTTTAGCTGTGAAAAGAATGAAAGTCTTAGAGACTACATAAATTAGACATAGACTTTGCCATTGACCC
CAGTGTTTGAGTAACACATGTTTTTCTGAGACCTAATGCATGATTTTGAATTTAAATACCAGACTTTCAC
AGTAGTTATAGAAGTTGATAACTTGTAGTTGATGATTTTCTGTATATCTAATCTAGGAAAAATTTCATGT
CTTAAGGTAAGATAGACATTACCTTATTTTATCAATTTTGTGTATATTTTTCTTTGTTAAATTCTGTACA
TTTTAAACATTTACTTGTCAGTTTCATACATATATATGTACTGTATATTATATCATAGTCATCTCCAGTG
CCCTCTTTTATTCCCTTCTATGATCTCCCTTTCCTATCTTGTCTTCTTCCTTCTTTCTCTTTGTCTTTCT
TTTAGGCCTACATTGTTTAGGGTTGCTTGCATGAACGTTGGCAGGGAGTTATCTACTGGAGCAATTTACC
AGTAACTACACTATTGAAGAAAATGACAACCCTGCCCTGGCAGCCATTAACCAGGTGTCTTCTCTCTGAA
TACCTTGTTCTTTAGGGACATGCTAGGCCAGTGCTCGCTCTACCATTGAGCTACATCTCTAAACCTTGTT
TGTTGAGAAGGGGTCTTCCTGGAAGTTCAATCTGGCCTTGCAATCACTGGCAGATCAGGCTGCTCTCAAT
CCTGGGGTCTTTCTGTCTCTGCCTCTACCTCCTAAGTGTCAGAGTTACTGCTGTGTAGATTTATATGCCT
GAATCCTAAATACTTTCTCTGAACTTTTGGTGTTTTCTTTTGTGCCTCTCTATAGTTGAAGGAACAGATT
CTGCAGGCTGATCAGACCTACAGTTCTGCCCTGGAAGGAATGAAGATGGAAATCTCCCAGCTAACTCGGG
AGTTGCACCAGCGAGATATCACTATTGCTTCTGCCAAGTGTTCCTCCTCAGACATGGAAAAGCAGCTGAA
GGCAGAGATGCAGAAGGCAGAAGAAAAGGCTGTAGAGCATAAGGTAAAGTCCATCCAGGGCGCTCCACTT
CATATTTACCTGTTTGTTTTTAAAGAATGAATTTATTCAAGGAATAACTTCCGTATTTCAGTGGTAGACA
CCTGCTTTGGGCTGGTTAGCAAGCAGGATAGTGAGGGTTGGTGGGGAGGTCAGATTGATACAGACAAGGA
AAATGACTACCTTAGTGGTACGTGGGCAAGGTACTGGGAGCACAGCCAAGGAGTTGCTTATCTAGAGTAA
AGATCTCTCTTCCTGCAAGAGGAGAATGAGTAAGCTGGAAGAGACCATGCAGAGCTAGTAAAACTGCAAG
ATGTGGCCTGAGCAGCGGGTGGGGAGCCACCATTCAAGGGAAATGGGGAATGGATGCGTTGTGGAGATAC
TTGGCTGCACGGTTGGCTAGGCTTTGTGTTTGATTAGACTAGGGTAACAAAGGACAGTAAGAAGCTATTA
CTGAGGGATGAAGCTTGAGTAGCCAAGTAATGCCCTGTAACAAGGCAGAGAATGGTTGTAGTGCTGTGGA
AGATGATGCATTTCTATGAAAGATGCCCAGGATAGGGAAGCTAGCAGAGGAGACATTAGTTTGGCACATT
TGGTTTCAGATAAAGCCTGAACTTTTATATGGAAAAATTGGTTAGTTGGCTCCACGGAAATCCTGAAATT
CAAGGGTTGGCCATTTAGTTGTGGAATTCCTGGATGAAGGTGGCATTTGAAATCCCTCTCCCCAGATACT
GCAGTTATAGTGAAAAGGCAATAGAGTATGCCTAAGAGTGACTTGGATCAAATTTCGCAGATTTCTCAGT
TGGAGTGAAGGAGTGGGATGGAACTTTAAAAGAAGTGGCAGTGAAGGAGTTTCCCAGACTGTGCCTGCTG
AGGAAGAAGGCCATGAAAGGAGGCAGGAATTTTTTCATAGAGATAGTCTAGCAGAAAAGCTCAGCAGAGT
GAGGGCTGAAAATAAATGAATGCTTTATGCAAGCGCAGTCCCACGTGGTGGTGGTGGCAGAAAGTTGATG
CGAGCTGGAAGCAGGGTGTGTCTAAGATCTTCTGTGACTGAATCTTTGTGCCTTAGTCCCATTCATTACT
GAGTGACATTTAATCACATGCACAGTGCTTTGCCTGCTAACACCTTTGACACTCTTAAGAAGCATATGTG
GGGCCTGGGAGACGGCTGAGGGAGTGAGAGCCCCTCTCTGCAAGCATAAGGACCTGAGCTGGGATTCCCA
GAGCCTACATAGAAGCCAGGCGTGGCTTCATGGTCTGTTGATTAGCCTAGCATTGCAGGGGAGGGGCACA
GCAGAGAGAGCTGAATTCTGAGAACTTGTTGGCTCTGGTCTCTGCAAATACACATGACAGGCACACTTGT
GCTCTCATGTGTATGCAACACACACAACACCCTGTATCTACACACATTTTAAAAGTTTATCTATATGGAT
AGAGGATGTAGACTGGACTAAGTAGACTAGAGAAAATGATGTGGAAAAGCTTTGTAAAATATATTTGATT
TATGTGAGTCCCCCGATTTTCCTTTTGCAGGAGATTCTAAGTCAGCTGGAATCTCTCAAATTAGAAAATC
ATCGTCTTTCTGAAACAGTGATGAAGCTGGAACTGGGCTTGCATGAGGTACACACATCGACACTTAGCTT
TTGTGTTTTTGAAGCCTTTGAACGTAACTGTTAATATTGATGCTAGAAGAAGGTAGAATTAACTAGTTTG
AACAGTCCCTTAGGAGATACAGTCACAAATGAGGTCCAGGTGCCCTCCTGCCAGAGGATGTTTATGCTGA
AGCAGTAGGATAGTTTCCTATTGTGTGTGACAAGATGGATATATAGTCTGTGTTCTCAGCAAGGGAAGTA
TCTGTAAAGTGCCAGCAGAGTGCTGGCAGTCATTGTGAAGTGGACACTGGGCGCTGCTTTTAGTGCTAGG
CTGTTGTGCTCACAGTGTCTTTATTATAAAGCATACCATCCTTGAAGTATTTTAGTTTTCATTTTATTTC
TATGAAGCATACATAGTTCATTTATTCAACTATTCTAGAATACTCAGTGCTAAATTGCACACAGGATAAC
TGATCACGTGTTTGACTCTGAAGACATTATACTAATTTAGTGGAGAATATGAGAAATACATATAAGCTAC
TGTGAAATCAGAACAGGACAGTGTGAAGTTATATTGTAAAAGTCTTACAATTTAAAAAATGATGCATCTA
TTCTGATCCATCTTTTTTTGGTGTAGGTCAAAAGTCGGAAGATTGAATTCTTTTTTTCTGAACTTCCAGG
GAGTGTTCTGCTTCCAAAAAGATTCATAGTCACAAAAGGCACCCAGTTTGCTCCTAATAGTTCCAATTTC
ACAGTCATGGAGGCCGAGAGTGAAGACGCAAAAGGCTAGAAGAATACTTTATTTCAACTAGGAAAATGGG
GGACTATTTTCATAATCTTTCAGATGGGCAAGATTCTCTAAGCATCATGAAGCCACAAAGGATTTTACAA
CATAAAAATGTAAAATTTCTGGATGGCAAAAGGCTCCATTGAAAAGACTGATAATAGAAGCAAGTGTGGC
AGCTCATGCTTATAATCCCAGCTCTCGAGAGGCTGAGGCAGGAGGAGCGAGAGTCCAAGGCCAAACTGGA
CAAGACCCTGTCTCAGAATAAAAATAACAGCAGAGGGGCTGGGAGTGGAGCTTAGAGGGAGAGCACTCGC
CTAGCTTGAGGCCCGGGGCTGGCGGCACTGGTGCTTGAAAGAAAGGAGCAGTAACCTGGAAGTAGACACA
GGCTAACCAAGTGTCAGCAAGTAAGAGTTCCTACAGAGGCGATAGGAAAGAGACCCAGTCGGAAAAGGGC
AAAGAACTAAGTAGATATTTAAATTCAAGTGAGCGAGGGAAGCTATTAAACTTCCCTAATAGCCAAAATT
TTTAAAAATAAAAATTTCTTGCTAATAAGCTTGATAATGATTTAGAAAGTTAATATATGGCCTCTCTTGG
CAAGTATTTTAACAGCAGGCTCTCCCATGCATATAGAGATTGCCTTCAGTACAGTTGAGCCATGGACATG
GAGCTTGTAAGTCCATACACCCCCAGTCCTCCCGGTATTTCCACCTGTGATATTAATAGTCATATTCTTC
ACTGTCCTCCTGGTATTTCCACCTGGAATATTAGTAGTCACATTTTTCAGTGTCCTCAGCAGTTTGCATC
CCCAGAGAGCCCACCTTATGGGAGTAAAGGTTCAGAAATGGGCACAGCAGGTATTATGAAATAAACTGGT
TACTTAAATGGTTGATTTGTGATTGTACTCTGAAATATGCTGTCATTTAAGATGGAAACATTCATTTAAT
TTTTCTGCTACTCCACATCCCTGTGATACTGTAATGAAAAAAATTTTTTAAGCACAAACTCACATGGATG
GGAAAAAAAATAGAGGAGACAATAGCCAAACATTTAAAATATTTAATATAATTTTATTTTTGTGTGCATG
GTGGGTGATTGTGTACACGCATGTGTATGTACATGTGTATGAATGTATGAAAGCTAGGGGTTGATATTGA
ATGTCTTCCTTTATCATTCTCTACCTTTTGTATTGAGGCAGGGTCTCTCACTTGGACCCTGAGCTTGCAA
ATTGGGCTTGCTCCAGAGATCCTGTCTCCTTTTTCCAAGTATTAAGATTACTGGCAGTCTACTGTGTCTA
CTCGGCCAGTATTTACATGAGTGTTATAGATCTGAGCTCTGGTCCTCTTGCACGGCAAGTACTTTACCAA
CTGAATCGTTTTCTCAATCCCATAGCTAAATATTTTATAAGGTTTTGTCAAGGAGGTGGAAGATGGATGG
AGGTGGGGGCTGGGGCACATGGCTAACCAGCTGGCTGTCTTAATGACTAGATGGATGGCTAATAAGTGGA
TGGATTAGTAGGTGAGTGGGTAAATGGATGAGCAGGTAGGTGAGTGAGTGAGAGTGTAGCCAACAAATGA
CTTTGTATGCCCTGATAAAGCCAATACCGAGCGAGTAGTAATTTGTGGACTAATCTGTTTACACTATAGT
GTTCTTTAAAAATGTGTCATTTGTCAGTATTAGAGTGGCTCTGTTTACATTAAAGAACTGAGAGATTCCA
GAGATGCTAAAGGCAGACTGTGCAGGGTTCTGTGGAGCTGAACTTCTCTTGAACCTTAGTGGGAGCGCTG
ACCCTCTTTGCCCACTGAGTGCATGCCTGCTCCCCAGGTGTCATGCCGCCCTTCCCGCTCAGGCACCGGG
AGGAATTGTTAGTAAGCTAATAGCTCTCGGAGATTTCAGAAAGATGCACAGAAAAGACAAGTTTCTCTTC
TTTTTAGCTATGTGCAAGGGGTCTCAACCCTCCCCCCGCTGCGACCCTTCAGTACAGTTCCTCATGTTGT
AGTGACCCCAACCATACAATCATTTTTGTCGCTACTTCATAACCATAATTTTGCTACTGATATGAATTGT
AATCTAAATATTTTTGGAGATAGACATTTGCTAAAGGGGTGTAGGTTAAGGAAAGCTGGTTTAATGATTT
CAGACATCAAAGTTGTAAAAATCCTGTCAGCTTTAGCTTTCTCTGGTTTCAGAGTTGCATACAGTATGAA
TATCTGTACAGATTATCAAAACATAATTACTAGGAAATGCTTTTTGCATGTTACCACCCAGGAAGGTGAA
TTTACTTTTCCAAATTAATCTAATTATCTTAATGAAAGCAGCAGCTTTGATTAATGTTGGCCACTTTAAA
GATGAGTCGTAGTTCATAGCGTAGCCCTGCATTTTAGAAATAAAAAGGTGGGTAGGTAGTCGACAGTGTA
GCACTTGTCCTGTGAATACACTTCTCTGAGATATTATAAAACATGGACAGCTACTAATCCTAGTTTACAG
GTCTATTTACACACTTGTGAGTTGACAGGCAAATTATGTTTATCAGAACTCTATTGACTAGCTTACTTTC
ACAATTTTTCTTTACAGAAAATGAAATATGTTAAACTGTAAAGATCACCATAATTTTATAGAATGCTAAT
TAAATGAAAAAAATTAGAAAAAGAAAGTAGATGTGTATTATCAAGAAATAAGATATATGATCACAGAACT
AAGACCAGTTCATGGTAAATTCCTGCCACATAAAATAGTCCTGCTTTTATGTTGCTAACTTCATATCAAA
CTACATGCCAAAAAGTATTTAAGATGGAAGGGTATTTATTTTAAAGTTACCCCTTTTGGCCAGAATTCTC
TTTAGACTCTTGTGTTTAAATTACTTTCTTTGAGTTAGTTCTTTTAATTTCCTCCCACAGGTATTGTGTG
TAAGGAGAGGGCTCCTCATTCTTCTTGATTTCCAGGTCTGATTCTTGAAGCTTTCCTCTGCCTTGTTTCC
ATGCCTTGAATGCTTATGTACAGAAGGAAGCCATCCTGCCTTTAACTTTGTCCCTTCCTTCACTCTAGGC
AGACTGATGCTTTTCCTGTGTTCATCTTAAGAGGGCTAAAGACAAATCTTTACTTTCTCTAAGTAAAGCC
ATAATTAAAACTAAAACAGAAAATATCTCAGCCAACACTGTTGTAGGTTGATCATAATTACCCTTAAACA
TACTAGTGATTCTCGTGTTCTTTTTTATTAGGCAAAAGAGATTTCACTAGCAGACCTTCAGGAGAATTAT
ATTGAGGCCTTAAATAAATTAGTGTCTGAAAATCAACAACTGCAGAAAGATTTGATGAGCACCAAATCTG
AGCTGGAGCATGCTACTAACATGTGCAAAAAAAAAGATGGCGAGATCTTTAACCCAGCACACAGCAGAGC
TGCCGGGTTCAAGAATGCAGAGCTTAAGTAAAGTTCCCTCCCTCCCTCCCTCCCTCCCTCCCTCCCTCCC
TCCCTCTCTGTCTCTTAAAGAATTACTTATTTATTTTATGTATGTGAGTACACTGTAGCTGTATTTGGAC
ACCGGAAGAGGGCATCGGATCCCATTACAGATGGTTGTGAGCCACCATGTGGTTTCTGAGAATTAACTCA
GGACCTCTGGAAGAGCAGTCAGTGCTCTTTTACTAAGCCATCTCTCTAGCACCTTAAGTAAAGTTTCTTA
AAAGTCTCTTTGAAGTGCATGTGCTACGGCCTGCTTTCCTACTTTGAGAAGACTTTCTGGCTTAGAGCTG
TGCAGTCATCACAGGCTCTGGTTTAACTCTGCCCAGTGTGCACAAGAGGCTCAGGTGGCTCAGCCTATTT
CATCACAGCTTTTGTTCTGTGCACCCTGAGTGTACATGTGTGTGAACAGCAGCTTGTTCCTGAGTTTTCA
TGCTGCCTCCCACTACCTTCATCACACCTGTTCCATAAGAAGTAAAACGTGGCTTTCTATAAAAAGCACC
CATTGCACTTTTTTTTAAAATGTATTTATTTTATTTATGTGAGTACATTGTAGCTGTTTTCAGACAATAC
CAGAAGAGGGCATCTGATCCCACTACGGATGGTTGTGAGCCACCATGTGGTTGCTGGGATTTGAACTCAG
GACCTCTGGAAGAGCAGTCAGTGCTCTTAACCACTGAGCCATCTCTCCAGCCCAAAGAACCTGCCAACTC
TTTTTTTTTTTTTTTAAGATTTATTTATTACATGTAAGTACACTGTAGCTGTCTTCAGACCACCAGAAAA
GGGCGTCAGATATCGTTATAGATGGTTGTGAGCCACCATGTGGTTGCTGGGATTTGAACTCTGGACCTTC
GGAAGAACAATTGGGTGCTCTTACCCACTGAGCCATCTCACCAGCCCCCCATTGCACTTTTGATCAATTA
ATTTTAGGTTGTTACCATAAGAAAGGTTATTTCAATGAGAAGCCAACTGACTTGAGTTTGTTCTGTGTAG
CCTGTGTGTTTTCCTACCAGAAGCCTTTGTTAGCATCCCCACCCCACTCTCACCCACGGTGACATTCTGA
GCTTTCTCTGTGCACGGGGTGCCCTGGCCCTGTTCCTTCTGCACCCCACTGGCAGCATGTTTGGCCGCTG
CTATGCTGGGAGCATGAGAGCAGAGCCCGAGGAGCGCTTGTCTCCGAGTGGACTGCTCATTTGTAAGCAG
TAGAGTCCTGTCGCATAGCCTCTTATGAACATGCTTCTTTCAGCATTTATTGGAAAGAAAAGTTGCTACA
GTTTTTCATTTATCTTGTTTTCTAATCTGCTATCCAGGCCAATCCATGGCCAGCACAGACATGACGGAAT
AAAGACCGAGCAGTACAAAACAGGTCACCACTCGCCTCGAGGACAAACGTTGGATAGCATAGACCCTGTG
GCCAGGGGTCCCAGCCCCCTGAGTTCTCACATCAGCCCCGGCAGCTCCACCGTCTCTCTGCCTTCCAACT
TTCTATTTGAGGCTCACTCTTTGCCTTCAGTGCTAGATATAAATGATGTCAACTTTTCTGACAGTCTGTC
TGACTGTATGAATGACCAAGAAGAGTTTGTATCTTCGGTATGAAAACTTTCTCGTGCTAATACTTATTTT
TATTTTTAATTTTAAGCTAAAGAGGGTGATTTTTTTTTTTTCTTACTGGGTCCCAGTCAGTACCCTGTAT
CACTTTCACATTTGTAGTTTTGCTTGTGAAGGCACATTCAACCAAGACAGCTTCAAATTCAGGTTCTAAA
ATACTTAGAATATGAGTGCAGGCATTCACTGACTTTAGCCTGCGCCTTCTTGCCTGCCTTTCCCAAGAGG
GCTGACCATGCTGCAGGGATTGAATCAAGGGCTCTGTCTCAAGCATGGAGACTGAGGAGGGAAAAGAGGG
GCTGCTGGGGAGGAGCTGGTGGCCTGGGGCTGAGGGTGGTGCATCTAAGGTGAATGGAGCATGATTTGAG
TACCTATGATGGCGCCTTACCCTCACCACTAGGCTCCAGTGTTCTTTACCTTCCATCGTGAAGCCGGTGT
TGGGCTGTCCATTGCCTGTTGTTAGCTGTGGTGTGTCTTTGTGAAAGTTTAGTAACCTTTGTTTTTGTTT
TTTTGTTTGTTTGTTTGTTTATTTTTGAATGTGTCAGCAATTCCATGGAAAGATTTTCTTAAAGTTTTCA
AAATAAATGTCCAATTTAACTTACACATAGTTAACAAAAAAAATCAACACCCTGAAAAGTACCTTTCTTC
TAAGTTTGGCAGCCAGCCTTCCCTGCCCTATGATGATGTGATTTCACAAATGTCACCTTTTCCAGCTAGT
GTGAGCATCACGCTTTTCCTTCAGAAGTCAGGTATCTGATGGTTGCGCTCTACAGGGCCAACACGGTTGG
TCCTTATTATTCATCACTTGCCGGTTTGACTGCCCACTAAACTGTACTGTGGCCCTGCTGTACCCACATG
TGGACACACAGAAAGCAGGAGAGCTGGCTCCCACGCTCCTAATAGAGGGAGCAAAGGTGGCCTTGTGTTT
CGTTCCAGTCTCAGTGTTCATGTGGTGATGGCGGGCAGCACCCTGCTGTGTGAGAGCCTTGATTCCAGCA
CCAGTTACAGTGGTAGCCGTGGGAAAGTCATTTCTCACTTCTGAAACTCATTTCTGTTTTGTCAAATAGA
GACAAGAGGGTGTATATATGTCTATACTGTGTGTGTTTATTTCCCCCGGAGGCAATGATTTGTGCTAACT
CAGCGTTTAAAGTGACTTCTAAAGTATAACATTAAATGAGAGCAAACTGCGTGTGCTAGATTTCCTGTAT
TACCTTCAAAAATCTGAGATCTGAAGTATCACCAGAATTGAGAGTTGCAGATCAAAGGCCATGGAGTGGA
ATTTTAAGAAAGGGAAAAATATTGATTTTTAACTGGCATTTAATTTGTTTCCTGTAGATTTTCAGTGCTT
TAAAGTGAGAGTAACATTCAGAGTTTTGAAATAGCAGGCGTTGGCTGTATTCTTCAGTAAGAGGCAGGCA
GGCATTTCTAGTCTAAGCCGAGGTACTCTCAGACAACTGACGACATTTCCTATACACTTTACACAGACAC
TCTTTGGGTCAGAAAGAAAGAGCTCTGTGGAGCTATGTTTTCAGAACTTGCCTTTTTTTCTTTCTTGGAT
TTAAGACGAAAATATCTCCTCAGGAAAAATTCTCCCTTTAACAGGGCTAGGCAGAAAATATCAAAATGTT
GGGGCATTCATTCCTGCTTCCTAACACATATCATGATTTGTCCCTCTTCCAGGGCTCGCTGCCCACCTCT
CCTCTGGGATCGATAGCTACCAGGTTTCTGGAAGAGGAGGAGCTGAGGTCTCACCATATTCTAGAGCGCC
TGGACGCCCACATTGAAGAACTGAAAAGAGAGAGTGAGAAGACGGTGAGGCAGTTCACAGCCCTCGTGTA
GCCCTGCACAGCCACAGACGGAAGACTGGATTATAAAGTTGTAATGAAGCAATGCTCGCAAGGGCGGCAC
TGAGGAGTCTGCAGACTGGGCACTTGGGTATTTTCACTGCGTTGTGTGGAGGACTGCTTCCATAGGAACT
TGTGAGAATAAACCGCTTTGCAGACTTTTCTCACATGAATGTTTATTACCACAGAGCAATGCTTTCTCTT
TCCGACTTACATGTGAATAGCTGTGTACTACTGAAAATAAAGATTGTACTATACAGTTTGAAACTAG

An exemplary cDNA sequence of the Cep63 gene in human chromosome 9, is provided directly below.

(SEQ ID NO: 15)
AGCGGAGGGCCGTAACTGGGGGGAGCTCCTGACTCCAACACCCCACCCAACTCCCTCCTAGGGAGAGAGA
GCTGTCAACCTCCAAAACCAACCCAGGGAAGGAGTGAGGTCGAGGCCAAAACTTGGACAATCTCTGCACA
TCTTGCAATTCAATCTGGAGCCCCTTTGCTGTCTTTCCTTTTCCTGAATCTACTCTCAATGTTGACAGCA
CTGTGCGATTTCTGTATTTGCCAGGGGCTTCTCGTGGGAGGTCGGCTCCAGCCCTCTCGAGAACCCTCCG
GAGACCGTGGGGATGAGGCAGCGCTCAGCCAGAGAAGCCCTGGAGCCCAGGACCAGCGCTTCAAGTGCAC
CGGGCGGGAGGCAGGGGCGCAGGGAGGGAGGCTCTGGACCGGCCGCCCCTGCTGACTATGGCCCCGGTGC
AGGTCAAGGCTTGGGCCCGGGGATTTCCTTTCAAAGGTGACTTAGAGCCGCTTCATTAGGGTAGCGCCCT
CCCAGGCGGGCCCTAATGGCGTGTTTGGCAGAGTCAGCGAAAAGCAGCGCCCTGCGGCTCCAGCGGCGGG
GGAGCGCCCTCTTCCGCGCCCTCGCCCTCTCCCGTGTGGGGCAGCTGCGCGCACGGGGCCATTTGCAGCG
CAAGCAGCCTAGCGTGCGGCCGCGGCGCCCAGGTCGGAAAGGAGGCGGCATCATGGAGCTGAAGAAGGAC
ATCAACGCTGTATCTATCGACATGCTGCTGATCGTGCACTCGGAGAAGCGGCGCGCCGCACAGGGATTCC
AAGGTGTTGGAAATGGAGTCCGAAGATGGCAGAAATTAGAAGGAAATGACTTTCACGAAAACTTGGTGGA
GAAGCAGCACCCTCAGCAGCCCCAGGTCATCACTTCCTACAACAGCCAAGGTTCCAGGGATTCTGGCATG
GATATCTCTAAAGGCCATTCTTCAGCCTATCCCAGGGGACAATTTAAAGTGGAGTAGGTGAGAAAGGCCT
CACTGAGAAAGTGACATCTAAGCAAGGAAAGAGGGTGTCACTCTGTGGCCCAGGTTGGAGTGCAGAGGTG
CGATCATAGCTCACTCCAGCCTCAAACTCCTGGGCTCAAGCAATCCTCCTTCCTCAGCCTCCCGAGTAGC
TGGGACCACAGGGACACAACTGACTGTGGAAGTCCACCCTCGAGATGCCATGCCCCAGCTGCTCAAGAAG
TTCTCCTTGGCTAAACGTATGTATGACACAGGTACTCATGGTGGAGGGCTGGCAGAGAGGATTGAAGGCA
GCTGGTTGGGCCAGATCCCAGCCCAAGCATCTTCTGAGGGAAGGAGAAGAGTGACATGGGCCTGCAAACT
GGGAAAAGGCAGAAGCATGACAGCTTTATCCTGGGAGACTCTTTTCTCTACACCCAGCTACCGCTGATTT
TGCATCTCTGGAACTAGTCATAGGTTTACAAGGTGATAAAAATGGAAACACAAGACCCCGGCAACCTGGA
GGGAAAGATGCCCATGCCTACCCCTGGGATCGATCTAGCCTGAAATCCATGTCCCTGGACCTGCAGCAGT
TTGAGAAATTGGATATCTACGCCTCACAGGTGACAGCCAAGAGTGGCCTAGACGAACTGGTGAGTGACCT
GCTCCAGGAGGCCCACACTGACCTGGAAAGGGTCCGCGCCATCTGGATCTGGATCTGCCATCACATAGTT
TGGAATGCAGCTTAGAGAATGAGCTCGCCTGGCACAGAGTATGACATTGCAGCTGCTCAGGAGAAGGACC
GCCAAGCCTTCAAACCCACTGACATCCTGCGGACCCAGAAGACCAACTGTGATGGCTATGCTGGCCTCTT
CGAGAGAATGTGCAGAGCTGCTTGAGGAGCGTGTCAAGCAGGCTCTAGGGATGAACATTTCAGAAGTGAT
CTGAATGGTGTTGGTCTCAGGGGAGGAGCAGACAGCGGAGACTGCAGCCAGTCTGTACCAACAGGACCAG
CCAGCACTGTATCTCCCTGTCCCCAGTGCCCCGAGATAAAAGCCATCAAGGCTGTTTATATGCTCGCCGG
AGTGCAGTGTATGACCGTGCCTGGCTACTCCAAGGGTTTCGGCTACCAGACAGGGCAGAGCTTCTCGGGG
GAGTTTGACCATGCCTGGAATGCTGTGTACCTGGAGGGAAGATGGCACCTGGTGGACAGCACCTGGGGCA
GCGGCCTGGTGGACACCATCACCTCCAAATTCACCTTCCTCTACAATGAGTTCTACTTCCTCACCCACCC
TGCACTGTTCATCGAGGACCACTTCCCAGACAACAAGAACTGGCAGCTGCTAAAACCTCCTCAATCTCTG
AGGCAGTTTGAGAACAACATGTATCACAAGAGTGAATTCTACAACAAAGGGATGCTGAGTGCCCACCCAG
AGACTTCCATGATCAGAACAGGTGAGCCCGGAGCAGGTGCCAGTGTGCTAGCAGCAGGAATGGAGCATTT
CGCACTGTCCTCCAGGAGCCCGCCTGAGACTGCGCTTTCAAGGCATGCCCTTCCAGACGCATGGCTTGTG
AGCTTAGCACAAAGCTTGTGTGTGCATGTGGATGAGTGGAGGCTGCTGAGGCAGCCCAGGCCATCAGGGC
AGGGAAGGGCCATAGGCCACATCAGCAGGGACAAAAGCTCACTCACACTAGCATAAACCCAGAAAGGGAT
GTGTCTGAAGGCTGCAGGTTTCCTAGAACTTGAAGGCTGAACACACAGCCTGGCATGTCACAGAAGCCAG
GGCTGGGCACTTCCTAGCTGGGTGAGTCTCTCTCAGTTCACATAGCAGGGAAAAGGACTACGTCACCCTG
GCTACACATAGCCTCTGGGCTCAGGTCCCCAACAGAAGCTACTGGCCTGCTGTGTTCTCTTCCCCAACTC
CTGCTGGAGAGAATACATGGCCTCAGCAGAGCTGGAATCTGGGTGTTGGTGGTCCAACCAGCAAGGATGG
GGAGCAACATGGGTCTCACAGGACAGGTGGGGGCAGTCCCACCCAAGACAGAGAGTTGGGGCAGACTTCC
TTAGGGAGGTGGCCACTGAGCTGGGTCTTGGGAGAGGAGAACAGGCCTAGTAGCTAGACAGAGATGGACA
TGTTCTAGGCAGCAGAGGAGAGGCGGGGACACAGTAAGCCCCCAGCCTCCAAACTCAGGCACCCCTGGAT
GAGGGCCAAGCTCCAGCACCTCTGCCTGGCTGGCTCAGTACCCCTCCCTTCTGGAGGTGGGACCCAGCCG
TTTCCAGCCCACTCCCCTGGGGCAGTATAGGATACCCAGCTTAGCCATCTCCCATGGCTGGCCTGGGCTG
ACATGTGCTCTCCCAACCTTCACCACGGAGCCATGGCAGCTGAGTATGTTTGCACACTCTGAGGCATACG
GGGGCTGCCCTGGAGGGCCAGAACTGGTCACAAAAAAGCAGGGCCTTCTCTGGAAGATTGTCTCTTATGC
CTCTCCAAGGCCTTGGCACCTGATCCAGTGCTGGGCACACAGCCTGGGCACGCTGCACAGCCCTGTAAGT
CGGGTCAGTCCAGCCTCCCACACCTCAGCAAAGGACTGCCTGGTGGCACCGTCTCTGCCACTGGGGCCTC
CATCCGCCCAGGACAGGAGGTCTGAGCTCACACTGACTATTAGCACAGGGTGCCTGTGGGTGGGCCCAGA
GCCCCTGGGGAGCTCCAACCCACATTGCAGGGCAATGGGGCAGCGGGGAGCACAGGTGTCCCCAAGTTAG
TTCCAACTGACTTGTCCCTTCCACCACACAATGGCTTCCTTTGCCAAGTTGCTGAGCTCTTGCAAACTCT
TCCAACTCCAAATACCTGGCAGCTCCTGCAGCTAAAATAACAGCGTGAAGTTGCTCAATTTGAGAATCCA
TTTCCTGTGCTTACAACAGATGCAGGGCTTGTTCAATGTATCATAGGCCGTGGAAATATCGGAAAGAACT
GACCAGGTCAAAGCAACACCAAACTGATGTCATTGAGTGGAGTTTGCCTTTTTAAGAGACATAAAAATAT
AAACACCAGATTCCTTAATTTATAGAAATCTAAACCTAAATATGATTCCATATATCTGAAAAAAAAAAAA
AAAA

Anapc13, or anaphase promoting complex subunit 13, is a component of the anaphase promoting complex/cyclosome (APC/C). Anapc13 is required for ubiquitin ligase activity of the APC/C and controls progression through mitosis and the G1 phase of the cell cycle. The cDNA sequence of Anapc13 in Mus musculus is provided in GenBank Accession No. BC028526.1 (GI:20306452), incorporated herein by reference. The amino acid sequence of Anapc13 in Mus musculus is provided in GenBank Accession No. AAH28526.1 (GI:20306453), incorporated herein by reference. The cDNA sequence of Anapc13 in Homo sapiens is provided in GenBank Accession No. BC005398.1 (GI:13529286), incorporated herein by reference. The amino acid sequence of Anapc13 in Homo sapiens is provided in GenBank Accession No. AAH05398.1 (GI:13529287), incorporated herein by reference.

An exemplary DNA sequence of the Anapc13 gene in Mus musculus strain C57BL/6J chromosome 9, GRCm38.p1 C57BL/6J is provided below.

NC_000075.6 (GI: 372099101: 102626296-102634244):
(SEQ ID NO: 3)
GTGACCCAGAAGAAGGGCGGGGCCGGGAGGAAGCCGACGCGCGCGCAGTGGGTAAGGGTTTTTCGCGCCC
GCGGCCCGGGTTTCCGAGGGTGGCCAAGCGTTCGCCCTCGAACTCCGGCGTCTACCGCTTCCAGAGGCGC
GGGCCGGTACTTTCAACCGAGCTTCAGAGCCACTTCTTTACTGCACGCTGTGCGGAGTGGAACGGAGCCC
CCATGGCGACGCTCGACCTCCCCCCTTGCACTTCCGCATGTAGTATCCTCCCACCCCTCGAACCGGAAGT
CGCTGCAGCGGGTGACTGTAGTGTCCCAGGATGGTGGCCTCCGAGGGCCATTTTAGTGTAGGCGCCGTGC
TCGTAGCTTAGAAATACATTTACAAAGCTGTGTGCATGAAAGGTGTGTATGTCTCATAGGACGTACGCAT
CTAATGTCTAGAAAGAGATATCCGGGCAATGTGACAAGCTTTCCTCCTGTTGTCACGTATTGTGCCCAGA
GATCTAGGGGACTGGTGAAGGAATGGACCCTTGGGACTGCACTCAGCCTGGGGTCTAAAGGAAACAATCT
GAGAGCTGAGAGTTTGATATTATCTGGTATCCGGAGCGGGGGAACTTTCTGTTTACGTAAAGGGTGAGCA
CCCGAGCACCTGACCCAGGAAAATAGATGTGTTTGGAAGGACGGCCAAGCATAAGTGATGAGGGACGGGG
GTGGGTGAAAAGCGTGCTAGTTTCTAAGCATGTAGAAGAGAGTCATGTTACAAAGTATTGAGGCGAACTT
GTGGAAAGACTGGGTATCCTTACATTACCGCTGGAGACAGTAGTACCCTGCCAGTCAGTCACAAAGCTGA
CCTGACTTGTTTCATAGATCATTTGAGGCAAGCAGCAAAGCATTCAAGGACAGAGCCTGCTGAAAAGCTG
GAGAGAAAGTCAGAGCACTTTAGAAAAAGGTGCAATCCATAGGGACTGGGAAAGTCAGCACCAGAAGGTG
TGAAATGCAGCCCAAGGCTCAGCTACAAAAACAAAAGGCTTGTCAAACCCTGAGGGGTGATTAGAAGTGG
CCCTTCAGCTGGCTTTGCGTTATCCACTAGCTAGGGTCTGGTTATGTCTGAGGAAAACTGCTAAGTTATC
ACCTGGTTTTAACGTGGCAGAGGGAGCTTCAGTGTGTGTGTGTGTGTATACGCGTACACGAAAGTTGTGG
TGGTGGGGGAGATTTCTGCGTACAGGTGGAGTTGTAGATGAGTGTAGAGGCCAGAGGTCAACATCAGTGT
CTTCCTTTATTACTTTGAATCTTAAACCTGGAGCTTTCTGATTCAGTTAAACTGCCTGGCCTGGCAGCTT
CCCAGAGAACACAAGTGTGCCATCGTGCCAGGCTTTTTCTGCGGGTGCCAGGAATCTGAATTCAGGTCTT
TTTGCTTGCACAGCAAGCACTTCACTCCACTGAACCTCCTCCCAGGCCCCAGGTTGTTTAATTTTTTAAA
GTAAAAAGCCCTTTCCCTCCTACAGACTAGGTAGATAGCGCTAGCTTGCTTGCCTTTATTCTGTTGGAGG
CAAGCACATAGCCGCGTAGCACAGTTTGTTTCTTGTATGAGTCTCCCTTCTCTCGGAAACAGAACAGTTC
CTGAGAGAGCCACTGGTACTAATCCTCACAAGACCTTTCCTTGGGTTGCCACTTTGTGGCAGGGAGCACA
GTAGGGTGTGTTTGTGATAAGGAACAAACTTGGAAGCCAGAAACCCAAAAGCACTGCTTGATGACTGAGT
TAGACTGCAACAGTTTCAGGAGAAAAATGTGGAACAATCTGTGGTCTGGGAGCTTAGCAAGCATCCTGAG
CACTTGGACCAGGCACTGAAGAGAGCCAGCAAGATGGTTTAGTGGCTTAAAGGCGCTTGCTGCCAAGCCT
GGCAACCTGAGTACAATCTCTGGGACCCGCCTGGTGGAAGAAGAGAACCAAAATCCCCAAGTTGTCCTTT
GACTTCCACACGAACTTACACATATACATTGTTTCTTTCAAGAAGGAAGTAGGCTGGGTGTTGGGATCAC
AGCACAAAAGTTCTTGCACCCTCACATATCCAGGGAGGCCGAGATGTTAAGCATGCAGGATTGTTCTTCC
AATGTGAGAGATATAAAACTTGTTCAGTCCACAAAGCTGGGAATTGGAAGTGATTGACAGCCTGGTGATC
TGCGTTATCTGTTGGGCCAGGCCACATCAACCTACAACCAGGACCGAGGGTGGCTAGTAATTTAAATGAC
CCTGATAAGCAGGGGTTCCCTAAGCTCCAGAAGTAGCTAAGAGACCAGATGATGTCTATGTTATTGTATG
ACCTTCCTTAGACCCTTAAGCTAGTACAGGTAACCTATCTCTAGAACCCATCGTCTTGGAAGAAATAACA
TACCCTAAGTCGAGTTTCAATGTAATTGCACCTCCCTGTGCACCAGAAACTGTTATTACACCAGGAAACT
TTCTTCCAAATCATACTATGTTTAAGTTTGTTCGGAGTAAACCGCCTAGCATCAGACCCCCCAAGTCTAA
GCAAGACTGATAAAGTCAGTCTGAACCCAGTTTTCATTCTCACCTGACAGTTTGCTTCCCTCCCTGGACT
CCTGCAGGGCCCCCTCAGCTGGGGATGGAGGTCGGTTGGCAGTGTGCTTAACTGTAGTATACACAACACC
CTGGTTCTGTAACCAGAGCCGGTGGCACAGGCCTGCAATCCCATTGGTGGAGGCAGGAAGATTGGAAGCT
TAAGGCTGCTTTTGGCTATAGAGAGTTTGAGACTCTCAAAAAAACAAACAAAAAGGAGGACGCCCAGAGG
GATAGAGAACCTGGAGCTGCCACCGTTGATCTTGGATAGTTCTGTTGAGTTCAGTCTGGATGCTGTTTAA
GAAAGGAAGTGGTTTCCTGGGTAAGCCCTGCTGGCCACGTTGTTCCTGCCCTGAGAGTTAGCAGCTGGCC
TATGGTTGGTCACTTCACACATCCCAGCTCAGTGTCACCCAGCTGTTCCCTTAGCTCCTGTGCCTTTAGA
CTGCCAATGTTTGGGCCAAGACGGGTGGAGACTTGTGCCAAATCGTAGGGGAGGGCTTCAAGAGCAGGAG
CTGACACTCAGGTAAGTCGGTCTGAGTCAAGTCAGAGACTCGTCAAAGACATCACAAGCAGGGAAGGGTG
ATTGTATAAGATGGTAAAGATAAAAGAAAGTTACATTTCCACGCAGTGTCTTCTAGGACCTTATTCTGAA
GACCTGTGTTGCTCTCTGGTTCCACTAGCAGCCTGGTGTGTTGATCAAGCATGTCAGCTGAGGTGAGGAG
AGAACTGGAGTCTTTATGTTGTCATCTGACACATACAAGATGGGCCTTTCCTGGGGTAATGTTAAGCACA
GCAACAAAACCAGTTTCAAGGTGGAAGTATGAGGAATCCTAGTCCTGGATTATACTATACCTTCACTGTG
TTAGAAAAAAAAAAAAAAAGACCTCTTTGGCTTACAGGCCTGACAAGATCAAAGCTGCAGGAGGATGGAC
AGTGAGGTACAGCGAGATGGAAGGATCTTGGACCTGATTGATGATGCTTGGCGGGAAGATAAGCTGCCAT
ATGAGGATGTCGCCATTCCACTGGTAGGTTGTGATTCGAGCTCAGGAGAAAATGTGGTGAGACTTAGTGG
AGGTTGGTGGGAAGTGAAATGCTAAGGCCCAGAAATCCTACTGCCTGGTGTAACTAGCTTCAATACAGGT
CATACCAGGGCTGGAGTATACCCGGATTTTTGGAGCCACAAAAGCCCAGAGGGATGCCAAGTGTGAGTTT
AGCACATCACACCCAGGAGCAAGGAGCATTGCTGGCTTACCAGTCTTTGTGGGTTTTTTTTGTTTTTGTT
GTTTTTATTTGTGTGTGTGTGTATGTATATATTTATGTATGTATGTATGTATGTATGTATGTATAGTGTG
AGAATGTCTGCCACCCATGTGCAGATGGGGAGGGTGACAGAAGAAGGCATCAAATTCCCTGGAGCTGGAC
TTACAGGTAGTTAGAGATCACCCAACACAGGTGCTGGAACAAAGCTTGGATCCTCTAAAAGAAGAGTCGG
TGATCTTACCACTGAGCCATCCCCCCAGCCCTTAGGATTTATTTCCATTTGTGTATGTGTGAGGGTCTGG
GATACAGTGGAGGAGCGTGTGAGTACAGGTGCCCTCAGAGGCCAGAAGAGGGTGTTGGGTCCCTTGGAGC
TGGAATTAAAGGCAGTTGTGAACCACCCAACATGAGTGCTGAAAACCAAACAGTGTCCTCTGCAAAAGCA
GTAGAGCCCTTAACCACTGAGCCGTCTCTCAGCCCCTCTGATGCAGGCACACAGCCAGCTGCTTACAGGC
AACCTTGCCTTTCTTCTTTGGCCTGTAGACATACAAGCTCTCAGTAAACCTTTTCTTTGCTGATCTCTAG
GAAACTCCCTTAAAAGGCTGTTGTTGAACCTTCTGTAATAAGTAACCCCCCACTTTTGCTTATGTAGATT
TAAAAAAAAAAACAAAAACTCATTGATGTTACAAAGGCCTAACAAAGAACAAGGCAAACAGAGTCTGGGG
ACTTTCTCTGTACAGTGTGACACACGCTCTGGAAGAGCAGCTAACACAGCATGACTTCCAGGAGCATCAC
CTGCACTGCTGTTTTACAAAGAACAGACTCCACTCGCTCACTCACCGGCACTCTCTTTCAAGAGTAAGAG
GTGGGATGTGTTCAGTGGATATTCAAGTCGCAGCTTAGTTCAAGTTCAGGACAATAAAATAATAAGAATC
ATAGCTACAATTTATTACCAGTTTTAAGCCATAGACTGAAATTAAGGCTTGCATTATCTGTAATCCTTAA
GACAGCCATACAGGGGAGGTGGTAGTGTCTCCGTTTGGCAGGAAAAGAGTAGGATTAGAGAAGTACACTT
ACCTCAAGTCTAGTGACTAGTATGTCAGATTTCATAAGCAAAGCTCAAAGTCTGTAAGCTTTCATCTGTC
CACCCATCCATCCGTCCATCCATCCATCCAACAATCACTTCTCCAAAGTCTACTGTGTGCCCTAAGGGTA
TATGAGCATAAGATAAAGTTCCTGTTTGGAGCCAGGGATCAAGAGAGGTAGGTGACTAACTAATGACACT
ACCAAGCAGGAGGGAAGGATAAGCAAGTGGAGAATGGCTGGAGGCCTGGCATGCAGTAGAAATGCAGAGA
AGATGTAATGCTTAAGAAAGAATCAGATGGTTCATGGTGCTAGACAGGGTACTGTAGCAAAGAAGAGTGT
AGCAGGCCCAGGGTTTGAAACACGTTGGGAGATGGTGAAGCAGTGGGCAGCAATTACCTTAGTAATTAGG
AGGCAGAAAGTTGGGCATAGTTTTTGAGAAAATGTTTGTGCCCCACTTCGACCTCCTTCTCTAGGTATCT
AGGGATGAAAGGTCTGTGGAGGGAAGCTGCCATAGCTCACATTTGTCTTTCAGAAAGTGGGGTCTGTAAC
ATTGACAGACAGCCTGATAGAAGAAAGGGCCAGGGGACATCATGTGACTGCAGTGCAGTGATCTAGGCAA
GGGAGGAAAACCACAACAGCACCAGGAAAAGTGCTGTGACTTGCTGAGCTCCGTACAGCAAGACCTAAGG
AACACTTACATGGTAGGGAATGAAGGGGGAGGTCCCTCTGCTGACTTCCTGGTGCCATGGTAACCTGATC
CTGTGGTCCATCCACACAGTGTTGTGATTCTAAAGGAATGTGTCTGTCAGTTAGCTTTTGCTGCATAACA
AATTATCCTGAAACTTAATGGCTTACAATAACAACTGTTTGGTCATAATTTTATTTCTTATTGGGGAGGT
CTGGTCCACACCAGTTCAGCCAACTAATCCTAGGCTTGCTGGTGCTGCAGTGTCAGGCAGCAGACTAGCT
GGAGCCCCTCTGCCAGTTAACAGGATGTCTCCAGGAAGTAGGGTGCCTGGCACTTGGCACACACCACCCT
GCAGGTCCTCTGGGACCTCAGCCTCAGGTTCAGCAGCAACAGGGAAGTGCAGAGCACTTAGGGCTCAGGC
CCAGAACTGCCCCACAGCTGCATTCTGCTGAGATGGGCAAGTCTGTGGCCAATCCAAAGGCACAGTCAGG
GTTAGACTCTGCGGCTTGGCAGGAACAGCTGCAGACAGCTGCAGCTGTTGGTGAATTTGTCCCTGTGGAT
ATTGTGCGTATGGTATTTTGTGTTGGGTGGGCATGTTTTGTCTAGTTTTACATGGTAGTATTCATCGCAA
GAATTAATAGTCACCAAGCAAACGATACATCATTTTAGGAAGCCTAGTATGCCATTAGAGAGCATGACAT
TAAGTCGTTCTTAATCAAAGGCCTGTTTATTTTTTGTGACCTGTAGATAGTTATTAAGGTCAGATTTCTA
AACGGACTAGAGAATGACAGTTCTCTGGCTCTGTTTACAGAATGTTGAATCTCACTTTACTTTCGGCATA
GCAGACGCATGTTGTGCAAACACATTGGGGTTTTCCTGTATGTGTGTACCTGTGTGCCATGACACACATG
TAGAACTCAGCGTTGTCTGCAGTGCCTTTACCCACTGAACCACTTTACTGGACCTGCTTCAAATACATCT
GTACATATTGTAGCCAAGTATCATGTTCCATATGTTGAACATAATTTTAAAAATAAAAATACCTCCACAT
AGTATAAATATAAGTGAGAAATCCTAACTTTGAATATGAAAGCATCAGTTTTCTAACACAAAGTGTAAGT
TCAGGGAATTGCACAGAAATACATGTGGTGAACTCTGGAGCCATGGCTTATCTCTGTAAGAAAATGGCTC
TTGGGGTGTGAGCTAGTCTTTACTGTGCTTTCCACTAGGGGGAGCTCTCACCTGTTACATTGTCAGTGTC
CATTATCCAGCCCATATTGTCACTAAAGAAAGCATTTTGTTGCAGCATTTTTTCTTCTTGATACAATTCC
TGTATTTGAGAACATATCAGCAAATCAGATTGGCCCTTAAAGCATTTTTCAATAGGTTTAGCATTGGTTC
TTGTCAGTGTTCTCAGCCACTCAGCAGTGTGTGCCCTAGAGTACTGAGTACCCTCACAGCATGCACTGGA
TCTAATGTCCTTACTCTCCCCGTCCCCAAGGCAGAGACATGGGGGTGAAAAAAAAAGCTCTTTTTTTTTT
TTCTCCAATTTTTGAGAGTGGAAACAAAAGCCCCCAAGGGTTTGAATTACCTCATCAACCCAATAAGCCA
GCAAATGAAGGCTGTCTGGGCCTCCAGTCTCTGCCCTTAGCCCTCCTGCCCCACTGCCTTTTATAAGAAA
ACCAGAGTCACAGGGCCAGCATTCCCATGAGGCTTCCAAGCAAGCCGCACTGAACAGACCTCTGATATTT
TCTATCCATCTGTTTGTTTAGGCTCTCTCTTTAGAGCATCATATCAGCTTTGCTGGCTGTTCCCAGGCCC
TCGGAGGCTTTCTTTCCTAATGCCTTTGTGTTTTCAGAAGTAACTGCAGGACCAGGTCCACACCTATAAT
CCCAGCTCCTGGGAGGCAAAGGAGGATGGGAAAGAGACTTGGGGGTGATCGGCTGAGAGTGTGGCTCAGT
GAGAGAGTGCTAGTGTATCTCAGCAGGGCCCTGAGCGCAGTCCCCGGCCGCGCAGAAACACAGTTCAAGA
TTTGGGGCCAGCCTAGGCTACCTGAGACCCATCTCAAGGATAGAAAAGACACTTCCTGATGGTCGTTACG
GCTGTGCTTCCCTTCCAGAGTGAGCTTCCTGAGCCCGAGCAAGACAACGGAGGCACCACAGAGTCTGTGA
AAGAACAGGAGATGAAGTGGACAGACCTGGCCTTACAGGGCCTCCACGAGAACGTCCCACCCGCTGGAAA
CTGATGCCGGGCTCCTTCCCACGGACGGAGTTTTCAAACATATGGGGATAAAAATGTTTCCTGTCGCCAA
ATGCAGATCTTTAGCAATAAATCAACACTCAAAAGCATA

An exemplary DNA sequence of the Anapc13 gene in human is provided below.

(SEQ ID NO: 16)

CAGGATGTACCAAACGTGGAAGCGGAGACGGGGAGGTCTGGGGGTGCCAC
GCAAGTCCCTTTCCTCCCGACACGAGTGCAGTAAAGAAAAACGGTTATGA
GCGTAAAGTGACAGCCTCGGCCGATGGGAATAGGGGGAAGTCCGACACTG
AGCAACGAACGCATTCCCGCGCCTCCAAAACCTAGGCCGGGGGCGCTGGA
AACCCTTACCGGCACCCGGCCACCGCGGCAGACGCTTGCTCCTGCCACGC
CCCCCCCCCTCCCCCGCATCACGTGTCTGCACTCGCTTTCCTCGGATTCC
CGGATGTGGTTGCCAAAACAAAGGGGATTTGGTGATGGAGGCTTTGTTAG
AAGGAATACAAAATCGAGGGCATGGTGGGGGATTTTTGACATCTTGTGAA
GCAGAACTACAGGAGCTCATGAAACAGATTGACATAATGGTGGCTCATAA
AAAATCTGAATGGGAAGGACGTACACATGCTCTAGAAACTTGCTTGAAAA
TCCGTGAACAGGAACTTAAGAGTCTTAGGAGTCAGTTGGATGTGACACAT
AAGGAGGTTGGAATGTTGCATCAGCAGGTAGAAGAACATGAAAAAATCAA
GCAAGAGATGACCATGGAATATAAGCAGGAGTTGAAGAAACTACATGAAG
AATTATGCATACTGAAGAGAAGCTATGAAAAGCTTCAGAAAAAGCAAATG
AGGGAATTCAGAGGAAATACCAAAAATCACAGGGAAGATCGGTCTGAAAT
TGAGAGGTTAACTGCAAAAATAGAGGAATTCCGTCAGAAATCGCTGGACT
GGGAGAAGCAACGCTTGATTTATCAGCAACAGGTATCTTCACTGGAGGCA
CAAAGGAAGGCTCTGGCTGAACAATCAGAGATAATTCAGGCTCAGCTTGT
CAATCGGAAACAGAAATTAGAGTCTGTGGAACTTTCTAGCCAATCAGAAA
TTCAACACTTAAGCAGTAAACTGGAGCGGGCTAATGACACTATCTGTGCC
AATGAGTTGGAAATAGAGCGCCTCACCATGAGGGTCAATGACTTGGTTGG
AACCAGTATGACTGTCCTACAGGAGCAGCAGCAAAAAGAAGAAAAATTGA
GGGAATCTGAAAAACTATTAGAGGCTCTGCAGGAAGAAAAGAGAGAATTG
AAGGCAGCTCTTCAGTCTCAAGAAAATCTCATACATGAGGCCAGAATACA
AAAGGAGAAGTTACAAGAAAAAGTAAAGGCAACTAACACTCAACATGCTG
TAGAAGCTATAAGTTTGGAATCTGTGAGTGCAACGTGTAAACAGCTGAGC
CAAGAACTAATGGAAAAATATGAAGAACTGAAGAGGATGGAAGCACATAA
CAATGAATACAAAGCAGAGATTAAGAAGTTGAAAGAACAGATTTTACAGG
GTGAACAAAGTTACAGTTCTGCACTAGAAGGAATGAAGATGGAAATCTCC
CATCTAACTCAGGAGTTACATCAGCGAGATATCACTATTGCTTCCACCAA
AGGTTCTTCCTCAGACATGGAAAAGCGACTCAGAGCAGAGATGCAAAAGG
CAGAAGACAAAGCAGTAGAGCATAAGGAGATTTTGGATCAGCTGGAGTCA
CTCAAATTAGAAAATCGTCATCTTTCTGAAATGGTGATGAAATTGGAATT
GGGTTTACATGAGTGTTCCTTGCCTGTATCTCCCCTTGGTTCAATAGCTA
CCAGATTTTTGGAAGAGGAGGAACTGAGGTCTCATCACATTCTAGAGCGC
TTGGATGCCCATATTGAAGAACTAAAAAGAGAGAGTGAAAAGACAGTGAG
ACAATTCACAGCCTTAAAGTAGCCTCTTAAAAAAATCACAATCTTGGAAA
TAAAAATAAACACCAAAGAGTTACTGTCATCTGAAGTAGCAGCTCTTTAA
AAACATGAAGAGATAAAATTATAAAAATGATACATCTAAAGCAGTGGTGA
AGAAAGCTGAAAAACTGATACTTTTGATAGGCATTTTCTCTGCACTGGTT
TGTTTAAAGGACTTCTTCCAGCAATAAGTTGAAAGAATAAACCACTTTGC
TAGACAAAAAAAAAAAAAAAAA

AmotL2, or Angiomotin like 2, is a protein that binds angiostatin, an inhibitor of angiogenesis. AmotL2 mediates angiostatin inhibition of endothelial cell migration and tube formation in vitro. The cDNA sequence of AmotL2 in Mus musculus is provided in GenBank Accession No. BC027824.1 GI:20381294), incorporated herein by reference. The amino acid sequence of AmotL2 in Mus musculus is provided in GenBank Accession No. AAH27824.1 (GI:20381295), incorporated herein by reference. The cDNA sequence of AmotL2 in Homo sapiens is provided in GenBank Accession No. BC011454.1 (GI:15079242), incorporated herein by reference. The amino acid sequence of AmotL2 in Homo sapiens is provided in GenBank Accession No. AAH11454.1 (GI:15079243), incorporated herein by reference.

An exemplary DNA sequence of the AmotL2 gene in Mus musculus strain C57BL/6J chromosome 9, GRCm38.p1 C57BL/6J is provided below.

NC_000075.6 (GI: 372099101: 102717804-102733417):
(SEQ ID NO: 4)
AAGCAGATCATCGGCGCCACCGACTAGTGCGCACCTCCGTTGCTGACTGTACCTAAGCCGAACCGGGTCT
GCGTGCGCCGGCAGCCGCGGGCCGCAGCGGTAAGCAAGGTGCCGGGCACTCCGGGCAGTGCGCGAGGCCG
GCGGGGACGCTTCCCAGCAGCAGCGGGACACCGCGATCAGTCCGACCCTTTCTGGAGATCGGAGCTGGGG
TGGACGAGCCTCCCCGGGAGCGGGAGGGGAGACTGTGAACCCTTTCGAGGATCGATCCTGGGTGTCTAGC
CTAGACTGCTCGCGCTTTTGTGGGGGTGAGGACACCATTCTCCGACCCACTGGGGCTGGAGGAAGGACAG
AGGATCAGTGTTTGGGAGTGTGCGTCTCGCGTGCGGGGCAAGTGAAAAGCTGCGGTAGGCATATCTCGCT
GAATTATGAGGTTACCGTGGCTGCTTTGTCTGGATTGGGGATACAGGTGGCTTTTACCCTCAGCTAGCTC
AAGAATTCCTCTTTGTCTTCCTTGGAAAATCCTCTGCGGAGCCACGAACTGGCTCTCACTTAGAAAGAAT
GCAGCCTGCTGTCCAGTCCGGTGACGTCAGCGCTGAAGTATTTGGAAAACAAAGAGGGTTAGGGAGAGAG
GCGTACCCGCGATGCTTAAAGACCTCTCGCGGTTGCGGGTTTTGCTCGCCCGGGGCGCTCGGGCAGCCAC
CGGTGCTAGTGGTCTGCCTCGGTAGAGGTGAGTAACGCCGGGACCCTGGACTGGCCTGGTTGACTTGGAG
ACTAGCGTTGGTTCCTTTTGCTGGAGTGAGCCCAGGCCAAGGCCAGAGACCCGTGTTTGCTTAGAAAGGT
GGCTGGTTTTCCGAGTGCAGCGCTCCAGTGCAGCCCAGGTACAGCGCCCGTGTAAGGAAGTTGATTTTCA
GTAAGTGCTCTTATATAAGTATTTGAAAGAAAGATTGTGCATCTAGGGGATTCGATTGGGACGTTTGTGC
GATGAAAGGCCTCGGAAATGTTCGGTGCTCCGAGAGCCTGGCTCATAGTCGGCGCGCCTGGAATCTGCCG
GGAAGGCTCAGCATTCCTTCTAGCCGCTCCCGGATCCTTAGCGCGAGTCAGCCCAGACAGTGATTAACTT
AGGAAATACCGCGTAGAAAAAGCCGCGTGTGAGCAGACTCTTCAAGTCTTACACATTGCAGGGTGCAGTT
GGGGGGGGGGGGGAGGATTAGTTGCTGGCTTGGGTATCGTCAGGGCTCATTACCTTTCTCCAGCTCTGCC
AACATACCATTCCCAAACTGAGGTAGGACAGGGGGCTAGCATTAGTATGGCACAGAAAGGGGCCACCACC
CCCTTCCTTTGTCTCCCCCCTCGCCTCATTACCCCATCAACTGGCATAGGTAGTTGTTACTAATATCAAC
AGAGATGCAGAGGGATCATCCCCTGTGTGCCTCTAGCATACACGTAGGGAGGGGTTTTTCTAAGTAGCAA
ATGTGTGTGCCAAGAAAAGAGGTTGGTGGTCATGGAAAATGTTTCCCTGGCTCAGTTACTGCAAGCTCCC
CTCTGCCTCCTCACCAATTTCCATGGGGCTTCTTGTGCTGCTCACCTTGCAGAGTGGCTGCTAAGGTCAT
CTGTGACAGGGAGACTCAGATTTATTTTATCAGCAGCCTTTGAGCCCAGGGTCTGGCTCACGCGTCTTTA
TTCTAAAGAATCAATGGAATTATTTGCTTAGGGCTTCTTTCTGCTGGGCTACATTCTTTCTTTACCGCCT
GCCAGTAAACTGGGAACCCTATTGCTAAGTGGTTGAATTTGATGGACGCCTGTCCTACAGTTTGCCCATG
AGATTGTTTGGGGTAATGGCTGACTGCCGGGGACTGTGATATGAGATAGCCTTCTGGTTTTCTGTTGTAT
ATCTGTAAGATCAGCTAAATCTCCATAAATGTTACAATGATAAGCATTTGCCAAGGATTTATAGCATACC
AGGCGCTCAGCTTCTTACATTTCAACTCCAAGAGACACACTGTGTGTGTGTGTGTGTGTGTGTGTGTGTG
TGTGTGTACATATATATGTATGTATATACATATATGCATATATATATTTATATTAATGGCAAACTAAGGA
TCAGAAGCTCAGTAACTAAACTTAATCAATAGATGGCCATATTAGCACTTGCATCTGGGCCTGTGTAAAA
CCACCCACCTACCCACCCAGCCTCCTCCTTCATCCAGAGTGTTCCTGCAAGTGCCCCTCCCTCCTTTGTT
TGTGCTTCCTTTGACTGCTATTCTTTCTGTCTCTTAGGTGCTCTGAGACCAGGTGCCTGCTGGCCATTGT
CTAGGCAGGTGTGTGCAAGCCAGAGAAGCATGAGGACACTGGAAGACTCCTCAGGGACAGTCCTGCACCG
TCTCATCCAAGAGCAGCTGCGCTACGGCAACCTGACAGAGACTCGCACTCTGCTGGCTATCCAGCAGCAG
GCCCTGCGGGGTGGGGCTGGAGCTGGGGGCACGGGGAGCCCCCAGGCTTCCTTGGAGATCGGAGCACCCG
AGGACAGTCAGGTGCTGCAGCAAGCCACCAGGCAGGAGCCCCAGGGCCAGGAGCATCAGGGTGGAGAGAC
CCACCTGGCAGAGAACAGGCTGTACCGGCTGTGCCCACAGCCCAGCAAAGGAGAAGAGTTGCCCACCTAT
GAGGAGGCCAAAGCCCATTCGCAGTACTACGCAGCGCAGCAGGCAGGGTCCCGGCCGCATGTTGGGGACC
GGGATCCTAGAGGAGGGGTGTCCGGAGGCGGCCGGCGACAGGATGAAGCTCTTCGAGAGCTGAGGCATGG
CCATGTGCGCTCCTTGAGTGAACGGCTTCTGCAACTGTCCCTGGAAAGAAACGGTGCTCGGGTCCCCAGC
CACATGAGCTCTTCCCACAGCTTCCCTCAGCTGGCCCGCAGCCAGCAGGGCCCCCAACCCCGAGGGCCCC
CAGCTGAGGGCCCAGAGCCCCGCGGGCCACCACCTCAGTACCCACACGCTGTAATGGCTCAGGAGACTGC
GGCTGTCACTGACCCAAGATACCGACCCCGAAGCAGCCCACACTTCCAGCATGCCGAAGTCAGGTAACCC
CAGATTTCTTTCTAGTTCGGTCTCCCAGGAGGAGGAGAACCCCAAGATCATATATATCGTGGATCTCAAC
TGTCATGATCCCAATCCCAGGGCACCCCCACCAGTTCCTAGGGCTGTCTGATCTAGATCTACCCTCTCCT
GTCTTGAGAAGGGAGAGCTGGGGGGGCTGGAGGGTAAGGGGATTTAGCTGATCCAGTCATGGTAGCATGC
CTGTAATGTTTAGGTGAGGCAGGAGGATCACACCACCAGCCTGGACAACGTAGCAAGACTGTCTAAAACA
AAAAGAAACTGACATCATTGCTTTAACTCAAAATATACATAGCATTGTCTATAGCATTCAAATAAGGAGG
GCTCCCAGGGATGTGAATGGGGTTCAAAGGTGAGCATTTCTTTTTTTCTAAGAAAGAACTAACTAAAGAC
AATAGGAATCCTTACCAAACTTCTCCCGAGTTAAGTAGGCCAAAGGTGGAACTTATTGAAGGTTTTTAAG
AGAAGGTGGACATTTTTTGTTGTTGGCTCATCATTGGGAGGTGTTGCTATTGAGGCTGAAAGAACTAGGT
GGCTCGCTGGGCCACTTCTGTATAGTTACTGTCTGCCAGTTAATTACTTACTAGCTTTTCCCTTGTGCTT
ATGGGCCTAGATGAGTGAGTGAGTGAGTGAGTGAGTGAGCTGCTTTGGGGGGAAAGGGGGGGCTCAGGAT
GCTGTTGGACCAGAGTAGTGACTTGGCTTGACTTCCTGTGAAGTGAACTCTCGGGGAGTGTGTGTGTGTG
AACTCTCGGGGAGTGTGTGTGTGTGTGTGTGTGTGTGTGTGTGTGTGTATTGGAGGGCATGATATGATTG
GCTCTTAAGAGATCAATACTAGCCTTAGAGAATGGGTCATTGGAGCTAGGCTGGGAGCCCAGTTAGTATA
AGCAGGAAGCCAGATCATTGCAGGAGTCCATGTTCCTAGAAATGAGGGGCTGCCTTAGTCATCTTTATTG
GCCCCTGTGCCTTTTCCAGAGAAACAGGCACCATTGCTGTACCTCCTTTGGGGTGGGGTGAGGTGGGCTC
CAAGGAACCTGGCTGGGGACCCTCTGGGTTCAAAGTCAGTCACCCTTGCTGAATCCTTATTTGGTCCCTC
CATTTCCCACCAATTCGGTTTTCTTCACTCTGGAAGAGAATAGCCGCCTTGGGCCTAGTAGGGGAGCCAG
GGGAGCGGGGATGTGTGGGGGTGTGGGGGGGGTGGAGCCTCGTGGGTTTACTCTAGCCTCCACTCCCCCA
GACCCCCACCTCACCTCCCTTTTCAGGGCCGGTCAGGAAGAGTTTCTTCTTTTGGACCAGCCCTATTGAA
CCCTGCCAGGAAAAGATAATATCCAGGCAAGGAAGTGGGCTGTTTTGCAGGAGAACAAAAAGATAGGGAG
GAGGGCGGTGGGAAATGGAAGAACCAGGAGCAAGTGTGAACGGAAAGGAGTCGTCGCCATTCTTCCCTGG
AGCTCTCGATCATTCTCGAAGAATGTCAGTTGTGTCTGCCTGGCCTGTGGTAGGGAGGAGGGGGACAACA
GTTATCTGAGGCAACCCTAAGAGTGACCACACTTGGCAGCAAGGGGGAGTTTAGTCAGTCTAGTGGCAGC
CCTCTTCTCTGCACTCCCTGGAGCCTATAGGAGGAAAAAGGATGGGCAGGCTTTAGGGACTGAGTCAGGG
GCTCAAAGAACAGGGGAGCGAGCCATAGGCTAGAGGGGTGATTTTGGACACTGGGGATGGCAGAGGGGGC
ATGAGAGATCCAGAAGTGAAATCCAAACTTCCAAGAGGCTTCTAGAACATTCTGAGTACAACTGAGAAGG
CACTTGTGACACTTGGGGGTCACAAGGCCCCCAGAAAGAGCCTCTGGCTACCTGGGCCTAACTGTTCTCT
AAACATTAGAGTGTAAGGATGTGTTACACTCATGGCCAAGGGCCGAAAGAACACAGTCCTGAACTCTGCT
ATGGAGCTAATCCAGGGGTTGCCCCTTAACTGAGGTGATCACCAACAGAGATGGAAGCTAGACAGTCATG
GGTGAACCTGCTGTTACCAGCCATTTGGTCAAGCCTGGCTTCTGTGGCTGGAGACCCTTGTGATTTCGCC
GACACAGGTGCCACTTGGCAGCTACAGCACTGGTCTGCTGAAGGCCTAGCAGCTGGAGCTGAAATCTGTT
CCCCCTGGCATCTGATTCCCTGCTCTAACCTTAGCTGGGGTCTTGGAAAAGAATTTCGGTAGTTGTGGGT
AGCAAGACCCTTGAAACAAAGAGGAGAAAAAACTCAGGTGAGCCGTTTTAGCTGACATGGCTTACCTGCT
AGAATCCACTAGTAAGTGCCCTTCCTGGGGCCTGGGGACTCTGTGTGGCTTCCATCCCAAAAGATCCTTC
TATCAATTCAAAAGGTCATGAGCGCGTCTTTAAAGGTAATATTTAAGCTGTATTTGCTTAGGACGACCCA
GGTTGCTTGAACAGAATGAGCATCTGGATACAGCGCCACCGCCACCCTACCCCACACCCATCCTGCTCTC
TGACTCTTGAGTCTTAGGCACATACCTTGTGGCACAGGCCCTCCCTCTGGTGGCTGCCTAACACTGGGGA
GGCTCAGTCACTGGCAGTAATAGTCAGAGTGCGCGTGCACACGTGCGTGCTGTGTGTCTCGGGGCTCTGA
GCTTTATTCACCTCTTCTTTCCCAGGATCCTGCAGGCCCAGGTACCACCGGTGTTCCTCCAGCAGCAGCA
GTACCAGTACCTGCCACAGCCCCAGGAGCACTCTCCACCCCTCCACCCGGCAGCTCTGGGCCATGGACCC
CCAAGCTCCTTTGGTCCACCTGCAGTGGAGGGACCACCCAGTGCCCAGGCCACCTTGGGCAGTGCCCACC
TGGCCCAGATGGAGACTGTACTGAGGGAGAATGCCAGGCTGCAGAGGGACAATGAGCGATTGCAGAGAGA
GCTGGAGAGCACTTCAGAGAAGGCTGGCCGCATAGAAAAGGTGGGGACTCCAAGGACACTCAGAGCGGTG
GCCGGTGCAGAGGGAGGCAGAGGGGCAGGAAAGCCTATCAGGCTACCTTTTGCACCATATTCTCCTACAC
ACACCCCCAGACACACACACACACACACACACACACACACACACACATCCCAAGCTGACTCCAGACAGCT
GAGGACCGACCAAGAGGCCTGCCCTAATGCATGAGCAGTTGCTGGCCAGTAGTGGTTAGAAAGGACTCAT
TCACTAGCTCCCTGCACACTTGTACCCTCACCCTGCACAGCCCCTCCTGTGGACACCTCCCACAGAGAGC
CCTTTGTGCACCCACACAACAGCCAGTCTGTTCCAGGCCAACACTGGCCAAAGAGAAAGCCCCTCCTCTC
AGGGAAAAGAAAAAGCAACGCATTGGCAAAGCAAGCCAGCTAGGAGCACGTGGAAGACCTGTGGCTTCCA
AGTGGGAGGGGCAGCCACATGCTCTGGGCAGAGGCAGCTCAGGCTGGGGTCTGGCACCCCCACTTCCTCG
GTGAAGCCTGGCCAATTGTGTACTAAGGTTCAACTTTAGCAATAGCCTCAGGCTTTCCACTTGCTGTCTA
TCTACAGTGTTCTGGATAGGCTGAGCCACCTAGGTGCTCTCATCCCTGGTCCTTTGTAAACACTAGGCAT
GGGGCATGCTGGAGGTAGCCCCCTTGTAGAGCAGCTAGCCCAGTGTCTAGGCAGCAGTCTGGTCTACCTA
AGAGGCTGCAGTCCTGTGGCAATCTCAGTAAAGTCACTGGCCTCCCTCCCTAGGACCCTTCCTGAAGTTG
AATTAATTATCTGTCTCTTACAGCTGGAAAATGAAATCCAGCGGCTCTCTGAGGCCCACGAGAGCCTGAT
GAGGACCTCTTCCAAGCGTGAGGCCCTGGAGAAGACCATGAGGAACAAGATGGACGGTGAGATGAGACGG
TTGCAGGACTTCAACCGAGACCTTAGAGGTAAGGACCAGGCACGAGGGTTGGGGTGTTGGGAGGACATCA
TCTGTCAGACTTTGGGGGCTCTGTTTTCAGGGGGAAATTTAGAATCTGTTCTGAGCTATAGAGGCCTACA
TTAGCCCCCATCGTTGGGCCCTGAGCTGAGCTGGGACTTCCTTATAATCCCATCTCCCCAGTTTAGAGGG
TGCAAGCCTACAGTTAGGACCCGAGGGTAAAGCCCAGGCTGATAGCATCCCTCTATATTTAAAACATCTT
TTCTCCTTATTTGAAACAGAGAGATTGGAATCGGCAAACCGCCACCTGGCAAGCAAGACCCAGGAAGCCC
AGGCGGGCAGTCAGGACATGGTGGCGAAACTGCTTGCCCAGAGTGAGTAGGGGACCAGCTGAGAAACCTG
GGTGGGTGTAAAGGCAATGAGGCACAATGACGTGGCCCCACTACCCCTGCCACTCCCTTCCCCTTCAGGT
TTAACCCACCCTCACTACCCCCACCCCCCTTGCAGAGTGGTACAGATTCACTGAATGGCTGCTCTGGGAA
GGGGTGATTTCCAAAGATGAATAGGGACTAGGGAGCCTTAACTGGCTGAGCCACACACTATGGACTCTCA
GTCTCTCCTGTAGGGAGGCCATGACAGCCCTGGCAATAGATAAATGGAGGATGATGAGGGTTCTTTGGAG
TTTTCCTAGGAATGACAGACAACAGAAAGGCTGGGGACATGGGAAGCTGCAGGGGATGGTCCACATGGTG
AAACTTCCTGGGACAGCCACAGATGGGTTTAGTCAACAGCTACAGTCTGAAGGAGGCATTCAGAAAGTTT
CTCTTTAAAACAGGGAGGTCCAGGCATCCTGGAAGCTTTATAGACTAATCAATTTGTTCCTTCATTAGTG
GAGCCAGGCTAAGCACCTGGCCAGCGCTGGGCTGAATGTGAGAATCCATCCAGAGGATGACAGGGAGGTG
AGATGAGCTTAAGGCAGGAGCAGGTACCCACAAGTTCAAGGGAAAAAGTGTTCCTGAGCCATGTTCCTGG
TGATTAGGAAGCCCCTGGTGACCCTGGGAAATCTGGGAACAGCAGAAGTATGGAGGCAGTGGAGGAGGTC
TCAGACACTGAGGGGAAATGACAGGCTTGTAGGTAAGAGGTATTTAGGAGGAATATGTATGAGGGTACTT
TGTTAGGGGTGTCTAAAGAGGGAAAGACTCCAGAGAGAGGGGGTTTCTGAATGCTAACCGCCCCACCCCC
TTCCTTCGGGGACTTAGAGTGCTTGCTAATAAATAAGCCTTTGGCAGGGAATGGGCTCAGATTGGGTAAG
ACCTGTGCTATCCACTACTACCTTTCAAAATGAGGTGGTTAAAGGTCTCTGGAACGGAGGTGAAGGGGAA
AGAGGTGTGTCGCCTAGGGGTTTGCCCTTGGGAGTCTGTAGAAGCACCTACTGCTGTGGGAAGAGAGATT
AGGGGTGCTTTGGTGGGAGGGGTGGGTTTTGGAGCCCGGCTCTGACGGAGAAAGGAAGTCTTAAGGAGGG
TTTCTGGAAGCTTGCAGAGGGGGAGGGGGGCAGAAGCAGCCGGGAGGCGTTTCCTCTCCCCGGGGAGCAA
GCTAGCGCCAGGCTGTTCTTGGCTTCTTGCTGGAGCCATTAATCTCGCAGCCGCGGGTGACCTGGATGCC
TGGCATACCAGAGCTGCTGGGCTGTTCCGCTCCCAGCCCGTCCCCCCCTCCGTTCCCTCCCCTGTGCGCC
TGACAGTGGCGCCCTTCGGCCCCGGCGGCGCCCAGCCTGGCTTTGGGAGGAGAGAGTATGAATACAAAGG
GAGGCTAGGACCTGAATGTGGAGAGCTTGCTAAGATAGGCTCAGCCTCCCACCCTCAACCAAGGTATCTT
AGAGATTGTACAAGGCATCTGGAAACAAAAAACACTGGATCCACAGCTGGAGGAACTAAGAAACCCCCCT
AGCATTAGAAGGCTGTTTTGCTTTGTTTGTTTTGTTTTTCTATATAATCGCCATTTCTAAAGGGCTGAGG
TTGACACTTTTTTCTGGAGGGTACAGAATTCCTCCCCATAACTGGCTTTCCCCAACAGCAGCCCCCATCT
CTGCCTGAATTGTCTGATATGGGCCTTCAGGAGCTGACTCTGAGGCCTGACAACTGGTCCTGCCTGCTTT
TGGACAGGTGTTGCCTTGGCCTGGCACAGGCTACAGAAGCATCTACCTCCTAGTACTGCTAGGTCTGGAG
TGAGGCCTGGACATTCAGACACAGGCTGTAGTGCTAGTGGGTGAGAGAGGATGCTGTTTACGGGTCTGAA
CCTGGCTTCTTCCCTCCACTCCACTCACCAAAGGCAAGCCAGCAGAGTATGACCAAGTCATGTGTAAAGC
ATCATGGAACAGCCCCGTCTTTTCCCTCCACACCAAAGAGTGAGGCAAGCTAGGCCGATTGTAGCCTTCA
GAAGGGCAAAGGAGATAGCCTAGATTGCCCCTTAGTGCCCCTTAATGCTGTTCAACGAGTTGGTGAAGGG
AGGCCTCTGCATACTAGGGCCGTGTCCTAGCCACACATAGCCCAAAGAAAGTTAACATTTCTTCTGTCTT
TCCAGCCACATTTTGAGAGTTCGGTAGCTGAGTGTAACCTTGGTAGTTACTGTTTTGGATAATGTAGAGC
CATCCACTGTAGCAGAAAAATCTCACCAATATAGTTTTGTTCCAAACCCTGTTCCCTACGAACTCTCTAG
AGAAATAAGCAGCCATTCTTGGTCGACTAGCCCAAGACTCCCAAAGGGCGAACATCCAGGTTCTTTCTTG
CCTTGGCCAGGCTAAATGGAGAGGTTCCTCTAATGTGCAAAGTGCTCGGCAATTGCTGGAATCTTAGACA
CAAGTCTTTGGACCAGCATCCTTTTCAGTGTGATGCATGGTTTGAATTCTCTTATGATGTTGCATGTGGG
CCCCCAGACCATGCAACAAAAGGGTCGGTGAACAGGAGAGGCTACCGTCCGCAGACTGTACTTAAGAGTT
CATTTCGGCAAGCCTATAGGGAGAAGGACTGTGAGCAAAGCCTTATAGCCGGAGCCTCCCAGTCAGCCAT
TTGGTTCCTATGTTTCTTCCTCTATAAATCAGGAGCCTCAAATTCCTCCTGGGGGATCAAGTGTGATAAG
GTTAAAAGTGCACGTCCTGCTTGCACAACGAATGACCTTGTCACAGTACCCTCCCTAATCTATAACAGGT
TGGCACAGTGTCTGCCTGAGCAGTCTAGTTATCATGAAGCTTAAGGAAAATATGTTTTGGTAATGGTCAT
GTCTGGCACAGTTCAAGCAGCTGAAAGGCCACTTTGGTTTATTTGTTTGTTCCAGCAAGGGTCATGGGGG
TCAGGCTCTGCCGTTTACTCTTACCTTAGTCTGCATCTCGCCGAAGTGCAATATGTAACTCGCAGACGCG
TCGAGGGGGAGAAGCAGTTGTCACAAGTAGGCAAAGGTGGCCGCCATCACTGACCCTTTCCTCTTTAATG
TCTCCTCGGACAGGCTATGAGCAGCAACAGGAACAGGAGAAGCTGGAGCGGGAGATGGCACTGCTGCGTG
GTGCCATCGAGGACCAGCGGCGACATGCTGAACTGCTGGAGCAGGCTCTGGGCAATGCACAAAGCCGTGC
CGCCCGGGCTGAAGAGGAGCTACGCAAAAAGCAGGCCTATGTGGAGAAGGTGGAGCGGCTGCAGCAGGCA
CTGGGGCAGTTGCAGGCTGCCTGTGAAAAGCGAGAGCAGTTGGAGCTGCGTCTGCGCACGCGCCTGGAGC
AGGAACTCAAAGCCTTGCGTGCACAGCAGGTAAGCCCAGAAGACCTGTGCAGCTGGCAGTACCGGGCACA
ATTCAAAGAGTCCCTCCCTGCTCTCTAGTCTTTGTCCTCTTTGAAAGATGATTGCTTCATGTTCCCCTTG
GCGCTGCTTGGTCCCTCTCAGGTGCTTTGTATAGCAAAGGCTCTGCCTCGAGCCCTACTGTGGCTAATAA
CAGAAATTTGGTACCTTAGTACCAGTCAGTAATAATGTTCGTGCAGAAGGCTTGACCACTCTAATCCATC
TTCCCAAAAATAGGAAGGAAGATGTCTGAGTCTGTGCTTGGACATAAACATCTCTTGTGTTTACCAACCA
AATAGAGTTCTGTCTCTACACACAACTTTACCCTGTCATAGAAGGGGGGCCAAGACTTTGTCCCAATGTG
ATTGCCACTCAAACCCAATTCCCACTTCAGAGAATTGCTTTTTAGTCGTATGTGTCCCTAGTCTCCTGAC
CACCAGAATTAAGGAGAGGATGTGAGGAGTTTTGCTATTCCCTATATCTCTGGGTGGAAGTAGGAAGGGC
TGTCTGTATGTGCTGTCCATGACTGAACAGAGGGATGCATGTGAGAGACTTCATGTGAGCCTGAGGAACA
AAGTCCCCCTCGTGCCAGCCTTTACCCTTGACCATGCTATTCCTTGCTCTACAGAGGCAGACAGGCACCC
TCGCAGGTGGTGGCGGCAGCCATGGTGGGTCCGCCGAGCTCAGTGCCCTGCGGCTGTCTGAACAGCTGCG
GGAGAAGGAGGAACAGATCCTGGCTCTAGAGGCGGACATGACCAAGTGGGAACAGAAGTATTTGGAAGAA
CGGGCTATGAGGCAGTTCGCCATGGACGCGGCTGCCACTGCGGCTGCCCAGCGCGATACCACTCTCATCC
GGCACTCCCCCCAGCCCTCGCCCAGCAGCAGTTTCAACGAGGGCCTGCTGCCAGGCAACCACAGGCACCA
GGAGATGGAGAGCAGGTACCACAGGGTCAGGCAGGGGTATCTGCGGTCAGGGGTGGAACGGTGCTACCCA
GTCTCTTCCCAGTGTCCTTTGTCCCTGGTAAATAGGCCCCACCCATAGCAGCTCTGTATGTTTGACATTC
CAGAAATTATAGACAATTGTTTCCCTGAGTGAATCCCTGGTATTCGCAGGCTGGAGGTGGGTGGGTACCT
CCTAAGCTCTGGCCACTTCTCCTTCTCTGTAAGAGATCTTCATCTGAGTCTTGCAATAATAGGTGGGCTT
CCAAGGCCAAGAGGTCCTGCCTGCCCCAGGGAGAGATGTCTGGGTTTCTTTGCAACTTCTGTGAGGTGGG
TGGGCTGGAGGAGCTGAGCCTGTGGGTCTGACAGTGTGACCTCCTCCCTGACAGATTGAAGGTGCTCCAT
GCTCAGATCCTAGAGAAGGATGCGGTGATCAAGGTCCTTCAGCAGCGCTCCAGGAAAGACCCTGGCAAGG
CCACCCAGGGCACCCTACGGCCTGCCAAGTCGGTGCCGTCCATCTTCGCGGCTGCAGTGGGAACTCAGGG
CTGGCAAGGGCTCGTATCCAGTGAGCGGCAAACTGATGCACGGCCAGCGGGAGGTATGAGGAGGCTTGGT
GTGGGAGGGAAAGGGGTAGGAATTTTGGAATGCATATTACCAGACTCTCAGAATACACGCCCACTGAGAC
CAGGGCTTCCTGACCCCCTCTTCTAAGGGATCGGGTTTAGCCTTTGGCTCCCGTGACTGCAACCTACATG
GGTCCTGCTAGTAGAGTGGATGGATGCTGGATACAGATAGATCTAGGCAAGGGTGTTCCTGAGCAAATAC
TAAGAAATCCTAGATTCCAGCTACAAAGAAGGGCCACAGAACATTGATGCCTATCCTGACCCCATTTATT
CCTATTTTAAAAGAGGAGTCTTGCTGGGCGTGGTGGCGCACGCCTTTAATCCCAGCACTCGGGAGGCAGA
GGCAGGCGGATTTCTGAGTTCGAGCCCAGCCTGGTCTACAAAGTGAGTTCCAGTACAGCCAGGGCTATAC
AGAAAAACCCTGTCTCGAAAAAAAACAAAAAAACAAAAAAAAAGAGGAGTCTTTTCATTCTATTAGACAG
CTTGCAAATTAAGAACTACCCAGTCCACCAAGTTTGCTTGGGGTGTCCTCCATTGGGACCGTGTCTCCTA
CATCAAGCTGTACCCATCAGTGTCCATTCTTTCTTACAGACCGGGTCCCAGCAGAGGAGCCTCCGGCCAC
AGCTCCTCTCCCTGCCCACACCAAACATGGCAGCAGAGACGGGAGCACCCAGACGGATGGCCCTGCAGAC
AACACCTCTGCCTGCTTGGCCTCAGAACCCGATGGCCTCCTGGGGTGCAACAGTAGCCAGAGGACACCCT
CTCTGGGTAAGCACCCCACTTGGTTAGATGAGGGTTGGGAACATGGTAGAGGTGCAAACTGGAACGCATG
GGCCCTGTTTTCCCCACGTTTCCGCAAACTCATCCACTGTGGAAAGCCCCACCGTAGCGAACTGAGGGTT
GTTGGAAGTTGTCTCAATGGGAGGCAGGGCCATCGAGCCATTTCAGAAAGAAGGCAACGCAACAGGAAGC
ACTCTAACTCCTCCAGAGTTGCCCTCACATCCTCCTGTCAGTCCAGGAGCTTGACAAGTGAACTCAATGG
TGATCTTGGGTTGGGAAGCTATTTGGAAGAGCCCAGAAAGGAGGAACTAAGCCTTTTCTCTTTCTACTCT
TGTTTCACATTTGCCCTTGCATAGCTGCCTGTGTGTGCCCCATGATCTCAGAAAAACCACAGGCATGACA
GGAACGCTACAGCCTGCTTCTAACCCTGGGAGCACCCTTCCACTGATCATTCAAGCTAGTTCAGAGTCTA
AACACAGGCATGGGCTTCCTCGGCAGAGGGGAGGGGGCACAGCCTTGTGCTTAGTTGATCAAGGGAAGAG
GAACCTAAGGGCTGAGGCAGCTTAGGGGAGGGGGTCTTGTTTAAAAAAGACCCTGTGTTGGGAGCTGAGG
AGAACAAGTGGATAGTACTGACAGCATACACTTGTCTTCTTGTTTTTTCTCCAGACTCTATAGCTGCAAC
CAGAGTCCAGGATCTGTCAGACATGGTAGAAATACTGATCTGAAGGAAGGAGGCAGTGCCTTGGGGACCC
CGAAGCCTGGCTCCCTCCTGGCATTCCGGCAGTTGTCTTCAAATGCTGTGCCTCACATCCTCCGACCAGT
GATGCCTTGGTGCATGGGGCTCAGAGAGGAGCACCCCAGGGGAGCTAGCTAGAAAAGCCTGCACCCCCTA
GAAGACTGCTTTTTGGCCTATATTTCTCCTATGACAATGAGGAAGGAAAAGCCAGACATCACCTGCAGCC
GGAGGCCTGTTGCTGGGGCAGAAAGGAGCTGAGCCCCCTCCGTCCTGCCTGAAGGAAACAGCGGATTCTG
ACGCCCAAGAATGGCTGGGCACCAGCCGAGCGGTCTGCAGGGATCGTTAGGCCAGCTATGGTGTTCTGTG
GCTAAGCATGGCTCCAGCCAACACTATCTGCTCTTCAGTCCCTGACTGACTTCCTCAGATCACTAAGACT
TGAATCCTTGGGAGGTGGGGGACGATGGGAGTGTGAGTCTTTCGGGAGCCACCAGCATGTCCCCTTGGAG
TAGGTTAGAGGTCACTTGAGAGGCCCCGCAGAGCAGTGTGTTTTGTATATAATTCTTTTTCCTTGGGAAC
AGAAGACTAAGCGTGCCGGTTCAGAGTCTCCTCCAGGGCTGTGCTGCCTGCCCCTGCTGCCACAGTTCTT
GAAACATGTGACAGGTCTACCAGATGTTCCTCTGCCTGCAGTTCCTGGCTGTGGATTTTGATACTGGTTG
GCTTTCCTCTGCTTTTTAAATCCCTGTGTTTCGTGCCCATGGGATTCTTATCACCAGCCTTTGTGAATGG
CTCCACAGCTCCTATCCCTGGGACATCCTGTCAGTTTGGTTATAAACTGAGCCAGATGTGATGAGCCACC
ACAAGCGGACATCTGTCATGCCCAGCACCAGGTTCTCTTTGAAGAAACTGGAGAAGTTGTTCTCATAAAT
GTATTCGTTCCCACCAGCAGGGACTGGGAGAGGGAGCTATGTTGTTAAAACGAATCAGTGAACTTAAAGA
AAAGTCCTCATTATGGTGCCCCCACCACAGAATGGCACCCTTAGAGGCTGGCTCCTCAAGTTTTGGTGCC
CCAGCCCTGAAGCAGGGCCAGCTACTTGGAATTAGGTGAACTACCCCCACGTGGCAGACCACCAGCAGCA
GTGACTTTGACTTCTGGTCTTAAAGCAACCCCCATTCAGCTCAAAAAGCGATTGGTGGATTTCAGATCTT
TTGTGTTTTTCTCCTCTGGTTGTTTTGTTTGTTTTTCTTTCACTGTTTGAAGTTCTGTCTGTTGTTTGTT
CCATTCCCATAGTTCTCTCTGCATTTCCTTTTTAAACATTTGCATTTGCTGGGCAATTGCGTATTTTTTT
AATTCCCTTTCCCCTATGTAAAGCTGAGAAATACATCTGGTTCATGTACGTTGTTTACAAAGATAAAAGT
TTTTAAAAAAAAAGGAGGGGGAAAAGAAGGCAAAAAAATATTGTGAAAGGAAAAAAAAAACAACTTAATA
TATTTTGGATTAATATTTGGTATTTCTTTTAAAGTATTTTTTTTGTGCTGTGAACGTTTTCTGCCAAAGA
CCATGACGTGTGTCTGTGTGTTTAAGTTATCATGAATATTTAAAGTGTAAACATGGCTGTTTTGTTACGC
CACTCTGTACCAGGATTGCTGCTGCATTCCGCCAGGTATGACAGTATTTTAATAAAAAAAGAGCTTATAA
GGAG

An exemplary DNA sequence of the AmotL2 in humans is provided directly below. GenBank Accession No. BC011454.1 (GI:15079242) (SEQ ID NO: 18)

GCAGGAGCAAGCGTCTGCCGCGGTGGCCGGGTGCCGATTTGACAAGATCA
AAGCTGCAGGAAAATGGACAGTGAGGTTCAGAGAGATGGAAGGATCTTGG
ATTTGATTGATGATGCTTGGCGAGAAGACAAGCTGCCTTATGAGGATGTC
GCAATACCACTGAATGAGCTTCCTGAACCTGAACAAGACAATGGTGGCAC
CACAGAATCTGTCAAAGAACAAGAAATGAAGTGGACAGACTTAGCCTTAC
AGTACCTCCATGAGAATGTTCCCCCCATTGGAAACTGACGCTTGGCTCCT
TTCTTGTGGATGGATTTTCTCAAAGTACACAGATAAAGCACGGTTTGTTT
CAGTCTCCAAATTCAAACCTTTGAGTAATAAATCAGCACTCAAAAATGTA
CACCCATTTAGTTTGTGGTAGCAAAGTGCAATGCGAAATTGAATGAGAAA
CTGAGATTTCTCAGTAATGGTGAATATTTCGCTCTTTAAACCTAAAACTC
TTCATTGAGTAGCTTATATTTGAACATGATTGGTTAAACATTTGCCTCTA
CCTCTGATTTTGCTTTGCTGTCAAAGTTTAACACCTTCCAACTACTTATG
TGTGTCCTGTAACACAGGTGATTGAACGTATGAGAGGGAAAGGCAAAGAA
AAAGGAAGCCAGACACTAGGAGAATTATTAACTTCTCATACTTCCCCACA
TTGAGAAGCATTCGGAGTGTATTTAGCCTGTAGATGTTGTGATATGCAAA
TATCCCATTCCCTGGTTACTGGCATTCCTAAGATTCTTCATGGTATTTTC
AAACTTTGGATAAATTTACAGATTAGAAAGATATCTGACAGTTAATCTCT
GTTCTCCTTACAAATTCCTTTTGTGCTGCTGGAAAGGATCTTTGGCTAGG
TGGATGACTAGTTTTATTCAAAGCCTTTTCTCAAAGCCCTTTCAGTTACA
ACCACCCCACTATGGAATCAGTATTTAGTTATACATTTGTATAAGAACCT
GTATTTTGAAAAACACATTCATGTATATTTATTCCTGGAATTATTTGCCT
GTTAAACAGTGTCTTTCATGTTCTCTCCCCAGATTGTAAACTCTGTAAGA
AGCTGCTTGTATCTGTATCCCTTGTTGAAACTCTGAAAACACTGAATAAC
TAAAATCTCTTCTCATCCTTACAAAAAAAAAAAAAAAAAAAAAAAAAAAA
A

Gm5627 is a predicted gene found in Mus musculus. The non-coding RNA sequence of Gm5627 in Mus musculus is provided in GenBank Accession No. NR_033301.1 (GI:287325394), incorporated herein by reference.

An exemplary DNA sequence of the Gm5627 gene in Mus musculus strain C57BL/6J chromosome 9, GRCm38.p1 C57BL/6J is provided below.

NC_000075.6 (GI: 372099101: 102739648-102756685):
(SEQ ID NO: 5)
GAGACAGCCAGGGCCAAAGCCCTGCCTGACAGCTCTAGAGTGGACCCCGTGCAATGTGAAGTCTCTGGGC
TCTGGTTTCAGTACTCTAGGCCCTGTGTCTTCCACACCAGCCTTCCCAGAGCTGGGCAATGAGTCCACCT
GGAATGTGTAACTGCCACGCTCGTCGCTGCACTTAAAGAATTGGCCTCTGCCTTCCCTGCCAAAGCAAAG
ATTCAGATCAGCAGATCCAGCAAGGCCAGACATTGACTGGGAATGGTCCCCTCAAGTCAGGACCAGCGGT
GCCTCCTCTTGGGTGAGACCCGAGTGTTTTTTTTTGTTTTTGTTTTTGTTTTGAGGACCAAATATCCATC
ACTTGACAGAGCCTTTATTAATGGTGATTTCATCGTGGTCCCAGTCATAGGTGACCTTGAGGGAATTGAA
TAGAAGGAAAACAAGAGACTTGGGGGACTTTGAAAGTGTTGGGACAGTAAATGACACAGGTCTGGAGAGT
GAGGAAGCCACAACAGGATGCTCTAAGAAAACCTTTCTTTCTTTCTCTTAAAAGATTTCTTTTATTATTT
ACGTATATGTATGTGTGGGTTCCCTAGAGGGCCAGAAGAAGGTGTCCAATGTCATGGAACAAATGTGATA
GAGTATTGTGAACAGCTTGATGTAGGTGCTAGGAACAGAACTCAGGTCCTCTGTAAGAGCAGTGGTTAGT
AAGCACGGGGCCTTTCTCTCTAGTCCTAGCAAACTTTTACAGCTAGAGAAGGTGTGGGGGCTGCCCATTC
CAGGATGTTCATTCCAGCATTGTTTAGTAACAGTGAATAATTGAAAATAACACCACGGTCTAAGCACAGG
GGACAGATGAAGAAATTATGGTTTAGCCATGCACAGGTGAAGCTGGTGGTTCACAGGTTGGTTGTCTGGC
TTTTAAAGGTACATTTGGTTTCTTGCAGAGAGAGAGAGAGAGAGAGAGAGAGAGAGAGAGAGAGAGAGAA
AGAGAGAAACAGGAAGAAACCGCTACCTCTGTGAGGAGGCTGTTCCAGGTTGTCAAATTCTCTGCCTCAC
CGTGGTTACTCACCACGGTCAGTCTCTATCATCACTGGGTGCAGACCTGTGCATGCTATTCTCTGGATCT
GGAACGTTCTCCCTGCCCCTTTCCTAGCCCTCTGCCCTGGTAAGTTCTTCTGACCCCTTAGGAGTTAGCA
TTAACATTATCCCTCTAGGGAAGGCTTTTTGACTGTCCCACACTGAGTTAGGTCACCCTGTTTCATGTTC
TCATTGTATCCCTGTAAAACTTCATTAGTGCCATTTAGGAGAGCATTTGTGACAACTCTCCCTCGCTTAG
GCATTCCACCTAATGAAGGCAGAGGAACGTGGAAGATCTAAAGTCACCGTCAGAAGCAGACCTCCAGGAC
CCGTGTCACCCTCGGATACCAGATCCGATGAATGAAAAGCTTATGAAGCAGTGGTATTCCCATAGTCTAG
AAATGATCTTCCTTGGCTAGAGAGATGACAGGGTGGGTGAAGGGGCTTGCTGCACAAACATAAAGACCTG
AGTTCGGATCCCAGAACCCAGGGAAAGGTAGGCTATGAGCTTAGAGCTAGGGAGAAGCTACGAGGATGTG
AAGTTAGAGGGATCTCTGGGGCTCACTCGCCACCAGCTTAGTACTAGCTCAATGGTTCTCAGCCTTCCTA
ATGCTGAACTCCTTTAGTGCAGTTCCTCCTGCTGTTGTGATTCCCCCAACGACAAAATTACTACTTCCTA
GCTGTAATGTTGCCACACTTATGAGTTGTAATGTAAATATCTGATATGCAAGGTATCTGGTATGTGAACC
CCTGGGACCCACAGATTGAGAACCACTGTTCTAGGTTCAGGGAGAGTCCCTTCCCCAAGGGAACAAGGTG
ACAAATTAGAGCAGAATACTCAGAGTTCCCTCTGGACTCCAACTTCCACAAAGATGCACGTGCCTATGAT
CAGTTGAATACTTCTCCCTACCCCCCCCCCCAAATGTATTGTTAAAGACACTATCTCACAATGTTTCTCT
GGCTGGCTTAGAACTTACTATGTTAACTGGGTAGACCTTGAACTCATGGAGATCCTCCTACCTATGCCTC
CTATGTGCTGGGATTAAATGAGTGTGTCACCAAGACTGGCCCCCAAAACATTTTTTTAATTACAAAGGAA
TAAAATTGTAACTTCAGAATGTGTGGGGCAGTGGGCATGGGGGAATAAAGAGAGTGAGAGAAAAACGGCG
TCCGGCCAGAGTTCTGGTGCTCTGGGCAGGTGGACTCAGGAGGACTGCCAGGGTGGGTGTCTGGCTGTGG
GAAGCCACAGAACTCCAGCCTGCAGTGGGGTGGACAAGGGGCGTCTCACAGAGGCCAGAGATAACAGGTT
CTCAGTCTCGGGCGTCCCAGACACCGCTGGACACGGTGGAAGAGCTGAGAACAAAGTGGGGCCGGTCATC
CCCAGGGCCGAAGGGAGGACAGGGCAGGGGGAGAGGGGGCTCTGGGGGGTTCTCACATGGGTGAGAGTCT
TTGGTCTGGTCTGTGGCTGGAGTGCAGGAAGGCCTTCCAGCGGGAGGTTAGATGTGGCTCTTTAGAGGAA
AGCTTATTCCATCGTTCAAGCACAGTGGGCCTTGATGAGCAGAGACAGTCTATGGTTTTAGAGCTTTATT
GTAGAGAGGCATGGAGAAAGAGAGGAGGTAGAAAGAGAGAGGCTGGCCATGGCCATGTGGAAAGAGGGGG
GAAGGGAGAGAGAGAAGGAGGACTAGAGATGAGAGTAAGAAAGGTGAGAGCTTAAGGAGAAAGAGGGGGA
GCCAAGCAGCTCCTTTTATAGTGGGCTGGACTATCTGGTTATTGTCAGGTAACTGTGGGGAGGAGTCTAG
CCAGAATGTCAGGAGCTTGGGGGCTTTATCTGTGTGACTGATAGTCACAGAATTATGGAGTTGGGGACCC
TGTCGTATCAGGCACCTGTCTCTGGGGATGTGGCTCACTGTTCCTTCCCTTGTAGAGTTTTCTACTGAGT
CTCCGGAGCAAGTCCCATTCAACCAAAACAGGCTGCCTTTCACAGCCCCACAAGAGTGGAGAAATCTTAA
GCCATGTGACCAAGGTAAACATCACCAGTCATTGGACATAGTGCCATCATGAGCCCTGTATAGAATGTAC
CAAGAAGGGCAGGTCACATCTGTGGCTCCCTTCCCACCAAGGCACAAGATGCATCTCCATATGGGAAAAT
ATCACACAATTGCAAATTCTCTGTTGTTTTGCTTGAGACAGGGCCATCTTATATAACCCAGGCATATAAG
AGCTCATGATCTTTCTGCCTCTATCTCTCCAGTGTGCTAGGGCTACACCGGCTAGGACTTTAAAAACAAG
AACCTTTCTATTCCATTTATGATTGGGGTGGGGGTATAACTTTCAGGAGTTGGTTCTTCTCCTTCTACCA
TGTGAGCTCCAGGACTCGAACTCAGGTTGCCAGGCTTAGTGTGGCAAATGCTTTACCCATTGAACTATCT
CCCAACCTTAGACCACTACTCTTAAAAAAAAAAGTCTTAAGGCAGCTGGATGTAATGGTACATGTCTTTA
GTTGTAGCAGTCAGGAGACTGAGGCAGGTGGACTTCTGAGAGTTTCAGGCCAGCCAGTTCAAAAGAAAAA
AAAAAGTTAGGGAGAGGTGGTTTAGTGATTAAGCGCACCTGCTGCTCTAGCAGAGCACCTCCATGGTGGC
TCACAACTGTCTGTAACTCTAGCCCCAGGGGATCCTCATCTATGGGCACCAGGCATGCACATGGTAAAGA
CATACATGGAGGCAAATCACACATACACATAAAAATAGATAACAGGTGTGCGCCACCACCGCCAGGCAGA
TTCTTAGTGAAAAGCTGTTGTGTGCAAGGATTTGCTTCATGTCTATGTCACCAGATAGCCTATTAGCTTC
ATGGAGTCCGAGGCCACAAATCCTGTCTTGTCATTGTTATCTCCTGACGTGATGTACCCAGTAAATATGT
GCTGATAAACAGATACAGTTACGGACATTGGGGACATGGGGAGTCATGGAAGGATGTGGAGGGAATGGGG
GAACATGGGAGACATGGGGGATCGTTGGGGGACATGGGGGCATGGGGGATGTCCATGATGCATTAACTGA
CAAAATAGGACATGTGAGGCCAGTTTTATAGCTTGGTTGGCATGACATCCTGGGCTCATTCATTCTCCAG
CACTGGAGGCAGGAGGATCAGAAGTTCAAGATTACCCTCAGCCACACCAAGGTCAAGGCCGGTCTGGGTT
TTGTGAGATGGTGTCTGAAAACAAAGAAAATTTAAACAACCAAAACCTATGACACGAAATAACTCATATG
ATCTGGGGATAAGAGCACACCCCTGTAATCTTAGTAATTAAGAGGCAGGAAGATTGCCAAAAGTTGAGAC
TAGTCTGGTCTATCCAGTGAGTTCCAGGCCAATCAGAGCCACAAAGCAAGACCAGTCTCAAACGCAATTG
CTTAATTAATTACAATTTAAAAGTAAACAAAAGCAAAAATACCTATGCCAATGTTTGCTTAAATAAAAAG
AACATAAAGAAGAGGGGTGGTGGTCTCATTCACCTTTAATCTCAGCACTCAGGAGGCAGAGGCAGGGAAA
TCTCTGTGAGTTTGAGGCCAGCCTGGTCTACAGAACAAGTTCTAGAACAGCCAAGGCTACACTGAGAAAC
CCTATCTCAAAAAGAAAAAAAAAACCATAATGATTCAGAAATAAACTGGGGACAGTGGCTGTCACTGAAA
GAAATAGTCAAAGGTGATCTTTATTTTGGTCTTAAAAAACAAAACAAAGGGCTGGTGAGATGGCTCAGTG
GGTAAGAGCACCCGACTGCTCTTCCGAAGGTCCAGAGTTCAAATCCCAGCAACCACATGGTGGCTTACAA
CCATCTGTAACGAGATCTGATTCCCTCTTCTGGAGTGTCTGAAGACAGCTACAATGTACTTACATATAAT
AAATAAATAAATCTTTAAAAAAAAAAAAAAAAAAAAACAAAACAAAACAAACCCTTTGTGCTGCACCTGG
GTCCCTACACACAGACCTGGAGCCAGTCCCTGTGGGGCGGGCAGCCGATGCAAGAGTATGAGTTCGGCAA
ACTGGTACCAGGATGCAGGATAGCCACAGGCAGCGTAGGGATACAGTACTGAATGGAGGCAGTAATCGCC
CTCCATTTCTCAGCTCTGTGATCCAGGCAGCTTATGTTTCCTGAGCACGTCTTCTAAACTGGCACATCCA
TTCCCAGGAGGTCGGAACTCATGGCCATTTGTTGTGTGGCTCACAGGAGTTTTTCCTCTCACAGTTCTGG
AGCCTTCTGAGGCTAAGATGTAGTCCAGGGTACACCACTCTGAATGTGTTGTGGGAGACGTCTTCTTGCC
TCTTCTGAGCTTCTAGCACTGTGGCTCTCAACCTTCCTAATGCTGTGACCCTTTAACACAGTTCCTCATG
CTGTAGTGACCCCCGAACCATAAGATTAGTTTTGTTGCTATTGCATAACTGTAATTTGTTCCTGTTATTC
CTGAATTATAATATTAATATGTGTCTTCCCATGGTTTTAGGAGACCCGTGTGAAAGGATCAAACTCCAAG
AGATCTCAACCCACAGGTTGAGAAACATTGCTCTAGAAGCTCTTGGCAACTTTAGTATTCCTCTCTTATA
ACTGAAACACGTCCATCCCTACCTCTGTGTCTCTAGATCTAAATCTTCTTCATATGCTCTTTCTAATCCA
GTGTGGCCATATCCGATTCCTTCTTCTTCCTCTCCTCCTCTTCCTCTTCTTCTTGAGTCTCACATAGCCT
AGGCCTTGAACTGACTATCCTGTCAAAGATGACCTTTGAACTTTTGCTCCTCAGATCTCCACCCCCTGAA
CCTTGGACTTACAGGTACATTGCACCATGCTGAGTTTATGTGGTGCTGGGGACAGAGGCCAGGGCTTTGT
GCATACTGGGCACAATGCTCTACAAACTTAGGGACGACCACCGCTCAGATGTCATCTGATTTTGATTATA
CCTTTGTTTTGGTTTGGTTTTTTGTTGGTTTTGTTTTGTTTGCTTGTTTTTTTGGTTTTTCGAGACAGGG
TGTCTCTGTGTAGCCCTGGCTGTCCTGGAACTCACTCTGGCCTCGAACTCAGAAATCTGCCTGCCTCTGC
CTCCCAAGTGCTGGGACTAAAGGTGTGCACCACCACTGCCCAGCTTAATTACACCTTTGAAGCTGTTTCC
AAATAAGTTATACTCTACAGGTAAAGGGGTTTAGGTCCTGAATATCTAATCTAATCTAATCTAACCCAAT
CTACCACTGATACCCATTTTCTATCACTGCCTATGGTGCGTCTCAAAAACGTAAGAAGCTGTTTAAAGTA
ACATTGTATCTACTCACAATTCAGGACTCAACAGTTAGGGCTGGGCTCAGCTGGAAAGTTTTGCTGATTT
CTCCTGAGGTCATTAGCATGGTTTTTTGTTTTTTTGTTTGTTTGGTGTGTGTGCGTGTGTGCATGCGTGT
TTGTGTGTGTGTGTGTGTGTGTGTGTGTGTGTGTGTGTGTGTGTGCGTGTGTCTCCTTCACAGCCTCTCA
GGCTCTCCTGGTGGATCAGATGAGGTCTATGCACACGCAGCTAGACAGAGGATAAAAGCATACGCTTCCA
TGCCTGCTGACCCTTCCAGCCTGACTGGGCTGCTGTAGGTTTTTATGGTCATGCAAGCAAGGCAAGTTCA
AATTCAAAAGACTCAACTTCTCTGTAGAAGTGGCAGAGGCAGATTACAAAGGGGCCTGCATATGGCAGTA
GTGGAAGTACTGAGCTGTCTTTACAAATGATCTCCGTAGACCTCCAGTTCTGGAAACATAAGGTAAACAG
ATCCAGGCCTTTCATGTTAGAACTCATCCTAGCAGCTGACCAGGCTCCTGTGGGGAAAGCTTGGTGAGTG
CGCATGGATGTGCAAAAGAAAGCAAGGTGCTCTATTGTGAGTGGCAGACAGCATGACTCCCATGCCCTAG
GCCCCTGGTGGCCCCAATCAGAGACTAGGATCATAATTTAAGTATAAAGCAAAGATGGCCCAACCTCAAA
CCCTGAAGAAACAGCTCAGATTCATATGCAATGCTCGCTGAGGCAAGATGTTTTATGACAAGAGCTGGTA
GGGAAATTAGACTGGAGTTACTCCTTGGCAGAGTCCCCTGTCCCTCCCTTCCTGCGGGGTGAGCTCTCTG
CCCCTTTCCCCAGTGACTGCTCCACAGAGCTCTGGCAGGCCACACTGCAGTGGGAGGGAAAGCCCTGGGG
GTATATATCGGATGTCATAGGAGGTGAAGTCCTGGCTGCACTTTTCACTGTGTGACTTTGAGCCTGTTTC
CTCATCTGTGAAATGTGGGCTCTCGGAGCTTTCATAAGAACCAAGAATGCTTTGCCAATGCTGCAATCAT
CTCTGAGGACCACACAGCCCCTGGCTTGCTCATGCATGCATGCAATAGTCAACCTTGTCTGCCGAGAGCA
ACATACTCTTCCTAGGACTTTTCTCTGCCTTCTTCATCCTCTGCCAGTCCTCTCAAGCCTGAGCTTTGAA
TGAGAACGCCATCTTCTTAGTTATCCTCTCCACTGTGGATCTTAGATTTCTCTCCTGAGGTTAAAACTTT
CTTTTTAATTATATTAAAATTCAATTCTGCCGAGCAGTGATGGCACACGCCTTTAATCCCAGCACTTGGG
AGGCAGAGGCAGGCGGATTTCTGAGTTCAAGGTCAGCCTGATCTATAGAGTGAGTTCCAGGACAGCCAGG
GCTACACAGAGAAACCCTGTCTTGAAAAAAACCAAAAAAATAAAAAATAAAAATTTAATTTTAATTCTTA
TTGGTTGAAATGCCTTTCATTTTTTAAATTAGGTATATATGTGCTGGCAAAGGAGTGTGTGCATGAGGGC
AGGTCCCACAGATCTAGAGGAACACGCTGGATTCAGAGTCCCTTGTGAGCAGCCACACCAGGTAAACCAG
GAGTGAACTCTGGTGTACCCCTGAGTCATTCTCCTGCCTGACTTTTCTTCTCTTTCTGTCTTTCACTCAT
GTGTCCTACCTTCCTTTCTATCTTTTCTTTTCTTTTACTTTTTTTATTGCATTCCCCTAAGATGGGGACT
GATCTGATGCCAGGGACTCCTGCAAGCCATCTACCACTGGACCACATCCCTAGCTCCTTCCCTGAGGTTT
CTCAATGTGAATGGCCAAAGAACTGGAGGTTTCAGAACCAGAAACCTCTAACTGGTTATGTGTCCCACCG
TCCTGGGGAAGAACCAGAAAGGAACGGAAGAGGCCACTGAATCCTGGGGCTGGGTCAGGCCAGCTCCCAG
CTGTTTCTGAAGACTTTTTCTAAAGAAACTTCCCCTTAACATAAACTTGCAGTGAAAGGAATTTTCACTA
AGACGGTTGGTTTGTTCAACGTTTATTGAGCAATTCAACCTGGCCTACCCTAGACACACAGCAGAGACAT
CGTGAGTCCTTTCCCCTGAAGAAAGAACAGGCTCAGAGTGTGAAAGGAGCTGTCACTCAGAAAGGACATG
CTGAACTCAGGGGAGGCTGCAAAGGCCTTGTGGAGGGAATGCCTTAAGTCTGTGGGTGAGTCTTTTTGGC
AGTGCTGAAATTCAAAGTGAGAAGTCTCACAAGAGACAGGAAATGTTCTGTTTTTATTTTTATTCATTTT
TTTTCTCTTCCCCCTTCCCTTTCTCCCTTTTGCTCCAGACCCACTCGCTCTGCCCCCGCCCCTCCCCTCG
CTCCAGGCCCTTCCCCGCTCACTCTGGGCCTTTCTTTTCTTTCTTTCTCTCTCTCTCTCTCTTTAAGATT
TATTTATTTATTTTTATGTATATGAGTACACTGTAGCTGTACAGATGGTTGAGCCATCACGTGGTTGCTG
AGAATTGAATTCAGGACCTCTGTTGGCTTCAGTCCCCCCTGCTCGCTCTGGTCAACCCGGCTCACTTAGA
CCCAAAGATTTATTTATTAAAATATCTAAGTACACTGTTAGCTGTCTTCAGCCACACCAGAAGAGGGCGT
CAGATCTCATTACGGATGGTTGTGAGTAACCATGTGGTTGCTGGGATTTGAACTCAGGACCTCTGGGAGA
GCAGTCAGTGCTCTTAACCGCTGAGCCATCTCTCCAGCCCCCTTCATTTATTTCTTAACAAAACCAAACC
AAATAGGGTCTCATGTAGCCCAGATCAGCTCCTGATTCTCCAAGTAGCTAGGGATGACCTTGAATGTCTG
ATCTTCTGGCTCAGCCTCCTGTATGCTGGGGTTACAGGTGTGTGCCCTCATAGCCATTTTTGGGGGGATT
CTGAGGATTAATCCCGGGCCTTATGCACAGCTACATTCTCAGCTCAGAGACTAGAATGGTTCAGAAAAGC
CTTGTTAGCTTCTGTGAGTGAGCAGAAAATGGATAGACAGTGCCTTGCTTGGAACTGGAGTGTCCTCATG
GACTGGGGCTCAGGCAGAGAAGAATAGGGAGTCATTTTATTCTTTGGATTTCTTGTTTCAGTTTGGCTTG
GGTTTGCTCTTGTTTCCATTTGATTTTCCCCAGATTTTTTTTTTCCTTCTGGCTTATGAGGGCAGTAGCT
CTGTGGAGGTCGTGGGGCAGCACCAACAGTCTAAACAGTTGTCGGTTCCATCCTACAGAGCTGCGTGAGA
TGGATTCTGACCACCTCGCTGTGGATTGGCGCAGCTCAGGAAGCAGTGCAGCTTGACACAGAGCTTGGGA
AGCACACCCCCATTGATGGTGCTTGCTTTGCAAACATCCCTGATTGCTGCGGCCTCTACAATCCTCTGAA
TGACGAACAACTTCATGGCCTCGTCCTTGGGCACACACCGGTCAACATCACTGCACAAGGTTGAGGCCCT
TTTTGACGTGATCGCTGTTTCTTTTTCCTGTCTGTCTTTCTCTCTCTGTGTCTCTGTCTCTGTCTCTGTC
TCTCTCTCTCTCTCTCTGTCTCTCTCTCTCTCTCGTCACCTTGTAGCCAAGACCTAAAAATTCTTGTGGG
TTTTGTTTTCTTTTACTTTCTGTATATGTGAGTTTTATCTGCATGTATGTCTGCATACCACATGTATGCC
TAGAGCCTGTGGAGGTCAGAGAGAACTTTGAATCCCTAGAACTAGAGTTACATACAGATGGTTGTGAGCC
TGCCCTGTGCAGAAGCAGAAAGGACTTGTAACTGCTGAGTTATCTTTCTGTTTGTTTGTTGTTTGTTTTT
TACAGATTTATCCTATTTTTCATCACATGAAAAATAAGATCTTGTTTTCTTAAGTAATTGCAGAGTGACA
GACACCAGAGTAAGGACTTAATGAACCAGGCAGAAGGACCCTTCATCTAAAGCAGCCAAAGAGTCGAATG
ATTAGAAGGGAGCATAAGAGAACGTGGTTAGCTCAGTGCAAAGGGATTACCAGGACACATGTACTCAAAG
GCATTGTTAAAGACCAAGGACAACACAGATGCCACCGTGAGGCAGTGTGAACAGGTGAGGCGTGAAATGC
ACATAGCCAGAAAGGCGGCAAAGATTCAGCTCCATCTTCCTGGATACTGCCCTGTTTGCCCATCCAACCC
ACAAGGCACCCGGTTCAGCTTCTGCAATAAGAGGCAGTATGATGCCCCAAAATGTTTGATACCAGGCTGT
GATAGCCCTGAGGACAAGATCTGCCTTGTTCCTGTGGCAGACCCAGCTCCAGCCTAGACCTGGCATTGGG
TAGAGGCTGGCAGTTAAGAGTTGCTAGCAAATAGATGGCTTCAGAACTTTCCAGAAAGCTAGTGGCTGAT
AGAACCAAGGAATTCCTTAGACCAGAGTACAGAATGGCAGGAAAGCACCAAAGTACACAGCAAGCTCAGA
CCAGGAGAGTTTGATCTCTGTGTCGAGCTCAGAGATGGAGAAGGACCCAGTAGGGTATCATGCTATTATA
CCAGAGCTACAAGGAGGTCCTGGTCCTGGATAGGAGGAACTCATGGGCAAAGGGCTAAGGGCAAACTAGG
AACCTAAGACCTGTGCAACCATAGAAAGGCTCAGTAGAAGGACCCAGATATAGCTCTCTCTTGTGAGACT
ATGCTGGGGCCTAGCAAACACAGAAGTGGATGCTCACAGTCAGCTATTGGATGGATCACAGGGCCTCCAA
TGGAGGAGCTAGAGAAAGTACCCAAGGAGCTAAAGGGATCTGCAACCCTATAGGTAGAACAACAATATGA
ATAACCAGTACCCCGGAGCTCTTGACTCTAGCTGCATATGTATCAAAAGATGGCCTAGTCGGCCATCACT
GGAAAGAGAGGCCCATTGGACTTGCAAACTTTATATGCCCCAGTACAGGGGAATGCCAGGGCCAAAAAGT
GGGAGTAGGTGGGTAGGGGAGTGGGGGGGGGAAGGGTATGGGGGACTTTTGGGATAGCATTGAAAATGTA
AATGAGGAAAATACCTAATAAAAATATTTAAAAAAGAAAAAGAAAAAGAAAGAAAGAAAGGCTCAGTAGG
GCCTGGGAAGATAGCTCTGTCAACTTGGGGCTTGGACTTTGATCTCTAAAACTCATGTTTAAAAAGTTGT
GCATAGAGCCAAGCATGGTGATACACGCTTTTAATCCCAACACTCAGGATGCAAAGGCAAGCTGATCTCT
ATGAATTTGAGACACAACTAGACTACAAAGCAAGTTCCAGATTAGCCAGAGCTATAGAGCGAGGGAGACC
CTGTCTCAAAAAGAGACGGGGGTGAGGGGAGAGGAGAAGGAGGAGGAAGGGGAATGGAAGAAGAGGAAGA
AGAGGAAGGAGAAGGAAGAGGAGGAAGAAGAGGAGGAGAGAGCTATGTGTGGAATGTACGTGTAACCCTA
GGGTTGGAGAAGCTGAGAAAGGTGGGTTCTTGGGGGCTCACATGGCAAGCTTTAGGCCAGAGAGAAACTA
GTCTCAAAAAAAAAAAAAAAAGAAAGAAAGAAAGAAAGAAAAGATGGTGTTTGAAGAAGCTGATTTTTGG
CTTCCACATATGTTTGCATGTACATTAGCATCTGCCCTCACTTGTGTTCCTACACCACACACACACACAC
ACACACACACACACAGACACACACACACACACACACACACACACACACACACATACACAGCAAAAGATCT
GAGGTCTGGTCCAGCTATCTCTGGAATGTTCATTATGGTCAGGCCTTGTATCCAGGCTCAGTAAAAGTCC
AAGAACTGAGTGGCCAAGATCCATACTTGGGAACCTGAGTCAGCCCTGGCAGAGTTGGTAGATACTGCAA
TCGGGTGGGCAGGAGCTATAGGCAGGGTCCCTGCGGCTTGTCAGAATGTTGCCGCCTGGGCCCTTTCAGC
TGGATGCTGGGTTGGTAGGGGAAGAGTACTAGTAGGAGCAGAGCTGTAGGCAGGGTAGAGCTTTGGGTCT
GACAAGGTCAGAGCTACTCCTGTTCTGGAAAAAGAAAGTCAGGGTAAACATCTGGCCCGTAAGTACCATG
AACAAGCACTCAAGGGTACAGGAAAAGGGTCAGAAGTGTGGCCTGAAAGGAAGTCGAAGCCATCAAGGAA
AGCCAAGAGTGTTGAAGCAGGGTTGAATGCTACCTTGGCTGTATGTGTGACACTAGAGAGGAGATGTAGG
TGGGAGTGCAGTCAGGCCCACCATCCATGATTCCAAAGCCCACCACACCTTGCACCATGCTCACTCTTTA
ACACCTTTGACTCAAACTCTCAACAGCAACCTCCGTGAAGGCCGAGGGGCATCCGAGAACAGGAGCAGGC
AAGAAGCCTGTGCCTCTTCATCCCTAGACACATCCCTTCTTGTAGACTGACTACTGGGCAGTAAACCGTG
AGGATAATGAAACCCAGAATCCCAGGATGGAGGAGGATGCTGCGTGGCTAGCAAGCTTCAACCCTTTTCT
AGAATGGAATCCTCTTTAGTAATATAGTGGGTTCTCAAGCAACTAGGTCTGGCCTGTAGGCCTGCCCCAC
CCTGCCTGATCCTCCCAAGCCCTGGCTTCCCGTTGGGCTGGTCCTACCAAGGTAGAAGGCTCCAGGGCCA
TATTTTGGGCCCTTCCCTCCCCTATACCTGCGTCAATGACTCTTTCTGCCTTCCACCCTAGCTTTAAGGA
GGGTGCTCATCGGAGGCTTGGCTCCTCATCAACAACCCAAGGCTCTCCAGCCTCATCAACTGTCAGGCAG
ATTGGGCTTGGCTGTGCTTCTGATCCAAGTTCTCCACCACTGCCTACCTCAGCACCCTCCAGTGATGTGC
AAGTTCCCAAGACCCACCTTAATGCCCCTCTGCCCAGCTCCAGTTGGTGGAGACCAAGCACATGGGCAGG
GCAGGAGTGTGAGACTTCCTAGCCTTTCCCCCTTGAGCCGACCCTTCAACTACCCCCTCACTTCATTCCT
ATGGAAAGACCAAGCCATGGTTTATAATACTTCCTCTGAACTCTTAGGAGGAAGATTTTTATAAAGCAGG
GACTTGAAGACTGGTAACACCATGTTCTGAAGGTTGCTGTAGTTTGCAATTAGTTCTTCACCAAAGCAAG
AAAGAATAACAACATCAGCCACAGGCCTGGGGGGTTGGGAGGGGAGCAGGGAAGCTTGAAAGCCAATTTT
TTTTATATTTAAAATGTTTTATTTTAAAATATTTTTATTATTTTTAATGATGTGTGGAAGTGTGTCTGCA
TGTAGTGTCATGTGAATCCAGGTGCCTGCAGAAGCCAGAGCCATTGAACCCCCTGGATTACAGCTACAAG
GGCTGTGAGCCACCAGATGTGGGTACTGGGAACTGACCTCAGGTCTTCTGGAAGAAAACAATTGCTCTTA
ACCTCTGAGTCATCTTTCTAGCCCCATAAAATGTTTTACATTTTTGTGGATGTCTCTCTTGCATGTATGT
CTGTGCCCAATTTGCATGCCCCATCTCTGTGTTGGACATAAGAGAGCACTGGATTCCCTGGAATTGGAGC
TACTGATGGTTGTGAGCTGCCAAATGAGTGCTGAGAATGGAACCTGGATCCTCTATAAGACTGACAGGTG
CTCTTAACTGCTGAGCCATCTTCCCAAGGCCAAGCATCATTGTCAGGAGCTACTGGACTGCCATGAAGAG
TAGACTGTAGCAGCTCTTGGCAGTTTTCCCTGGGGCTGTAGGCAGATGATAAAACCCACAGTATAGGAGT
AGATGTGCCCGACAGAGGAAGCAGAGACTCTATAGGAAACACAGAGCTGAGGCTGGGTACCTGGGAGCAG
CCAGAAGATCTCACAGTGCAAGAACATGCAGTAGATGCAGATGAGTTGGCTGCATAAGGAAGGAGAGGGA
GGACAGGAAATGGACCAGGCTGATCAAGATACCAGAAGAATTTCTATACAGCTAGAGTGAGGAGAGCGTG
GGTAGGACAGGCAACCCTCAGGGAACGCCCAAGGTTTGGAAGAAGACTCTTTGGCCAAGTGGTCATTTGA
TAAAGGGGGGTATGGCTTACCATTGAGGATCCTGGCCATGTCTCAAGAGAATACCCTCGAATACTCGCTC
ACTCCTGCTTTGACCCCATGAGCTTTAGATATGACTGAATCTGACACACACACACACACACACACACACA
CAAGGGGGGAGTATTGGATGTGTACAAGTGGTTTGCTGACATTCATGTCTATGTACCATGTGTCTAAAGT
GCCAAGGGGCCCAGAAGAGGGCATCAGGTGCCCTAGGACTGGAGTTACAGTCAGTTAGGAGTCACCATTT
GTGTGCTGGGGATCAAACCCAGGTCTTCCATAAGAGCAGCCAGCACTCTTAACCCCTGAGCCAACTCTCC
AACCCCACATGGCTGAACCTTTAAAGCCACTAGAGTGAGTTCTATCAGCTGTTCATCTGTCCTCCTGGGA
CAGAAGTGGCAATCCTGTATACTTCACTTTCTTAACCATGTAAGGAACTTATTTTGCAAGTAAGTGTCTG
GAGCTCAAACTGACCCAAGAATTTGACTGAAGTCACACAACACATCAGACACAGAGGAGGTTCAAGCCTG
TCTAATATCCAAACTCTGGGTAATGTAAATAGTTGATATATGCTTGTCACACATATATTCAACAGATTCC
CTGGGCATGTAAACTTGGGAAATGCTGGTTTCAGTGAACTTGAGCAGACTCTTTTCACAGCATGGAGAAA
AACTTTAGGCCATGGACTCTTGTCCCAGTTGGAGTTGCTGTCCCTTAGAAGCAGGACCTCCTGTCCTGTA
AGGTCAGAGGTGGGGCACTGGAGGCCTCTTACTTCAAGCTGTTTATATTCTCACTTTACATCTATGGCTC
TCCCCACAACACATACACATCTTATGTGTGAGGAGAGGGGACTGGTTTTTAATTAGACTTTGAAGAATCT
TGGGTGAAGGATAGTGGGGAAGCCAAACACCTCTGACCCTTAGTTGTCACAAGAGATACCTTCATTCTAC
GTACAGGGCCTAAGGCCTTGATCATCTATGGTGACATGTCTTTGGGACACTGCCAAACAACAAAGCCATT
TCATGTATTAAAGTTTGTTTGGAAGGGACTTGTGGATGTGGTATCATTTTAATAAAAAGCTGAGAGATTA
AGAAGCAGAGTTGAGGTGGAGAGAAAAGAGCCACATTTAGGGGAAAGAGCATACTCACATAGCCCAGTCC
TGGGAGAAGCCAGGAATGGAGCTGTTCTTGATGCAGCAAAGAACTGGATTTGGATCTGCAGGCATGAGAA
CTATGGGAGATGCTTGAGGCAGAGGTCATTTGATGCAATCTAGGAGTAGGGACTGGGATATGAGTCAGTT
GGTAGAGTGCCTGCCCTTTGTGCACAAAGACCTGGCTTGGGTCCCCAGTATCGCAGACACAGAGACTGGA
GGTTCGTGCCTATAATCCCAGCTCCTGAGGGGTAGAAGAAAAAGGATCAGAAGTTCAAAACGTTCTCTGC
TACATAGCACAGTCGAGGCCAGCTTGGAGGCCACACACACACACACACACACACACACACACACACACAC
ACAAACTCTTAAGGCACCCCCCCACCCCAGCTTCTCTCTCATTTCTTAATGGAATGCCAGCTTGCACACA
GCTATTCCACATCTTGCCTTTTTTCTCCCCTCCTCCCTCCTCCCCTCCTCCAAAGCCTCTTTGGCCCAGC
GTCTTTGTCAGCAGTGTTCCCTTAGGTTCTCGCTGTATGAAGCATCTTTCTGTAACTTCTGCTGGAGAAT
AGCTTCTGTGGACACCACGGAATGTCTGGTTATTGAGAAAGTGCGGAGAGATGACCTTCCCCCCTGAAGA
GTGCCATTGGCACACTTCCCTCGACCCTTTCTGCCTCCTGAGATCAGCCTTCCTGAAGTCCATCCCTTCC
GGGGAATGGGGCATGCCAGTAGGGTCCCCAGGGACATTCTCCCCAGCTATGCTGGACACTTCCCCACCCT
CTAGAGTAGATGCCAGAAGAAGAAGGGCAAGTGGAAATGGGGGTGGGAGTGGAGGTAGCGACAGAGTGGA
GACAGAGAAAAGGGTCTGACTGAGGCCTGTGACCCCTGCTGCCCCCAACCCATGCTTGTCATGAATAAGG
CCAGTGGATGGAGAAACACTAGTTTCTTTCTGGCTTCAAGGCTCTGGTTGGATTACTCCTTTGATGTGTT
TGTCCTTTGGCCTCAAAGGGACGGCTTCGACTTGGTAGCTCATGATAGCCTTAAGTACAGCAATCCAAGG
TCACCAGGCCCTCTTTCTAACACAAGTCCCCTTCTCCCTTGGCTTTCAGGAAGGACCAAGGCCCCAGAGC
CTCGTTAATCTGTTGTCAGAGTCCCTGATGGCATGCTAGGTATTTTTACAATTGTTCTAAAATCCCATTA
TCTCCACCGTCCGGGTTGCACAGGGGCCGCCATTGGCTGAATACGTGACCGACCACCAATGATTCCCTCC
AGGGAACGCCAGGCAGAGCTCCCTGGGCTGCTGGCCATCATCCAAACATGCTCCTCTCCGTGGAAAAGAA
GTGATGTCATACACGGAAGGGGTGGTCTCCACGCACAGGATACCTGTAAACACCGAAGCATGTTTCAAAG
AAAAACAAAAGTCAAATTACAGAGCTGC

An exemplary DNA sequence of the Gm5627 gene in human is provided below.

(SEQ ID NO: 18)

GTCAGGATCCTGCAGGCCCAGGTGCCTCCTGTGTTCCTCCAACAGCAGCA
GCAGTACCAGTACCTGCAGCAATCTCAGGAGCACCCCCCTCCCCCACATC
CAGCTGCTCTCGGCCATGGCCCCCTGAGCTCCCTCAGTCCACCTGCTGTG
GAGGGGCCAGTGAGTGCCCAGGCCTCCTCAGCCACCTCGGGCAGTGCCCA
CCTGGCCCAGATGGAGGCCGTGCTGAGGGAGAATGCCAGGCTGCAGAGAG
ACAATGAGCGGCTGCAGAGGGAGCTGGAGAGCTCTGCGGAGAAGGCTGGC
CGCATTGAGAAGCTGGAAAGCGAAATCCAGCGGCTCTCTGAGGCCCATGA
GAGCCTGACCAGAGCCTCCTCCAAGCGTGAGGCCCTGGAGAAGACCATGC
GGAACAAGATGGACAGTGAAATGAGGAGGCTGCAAGACTTCAACCGGGAT
CTTAGAGAGAGATTGGAATCTGCAAATCGCCGCCTGGCAAGCAAGACACA
GGAGGCCCAGGCCGGCAGTCAGGACATGGTGGCCAAGCTGCTTGCTCAGA
GCTACGAACAGCAGCAGGAGCAAGAGAAGCTGGAGCGAGAGATGGCACTG
CTGCGCGGCGCCATCGAGGACCAGCGGCGGCGTGCCGAGCTGCTGGAGCA
GGCTCTGGGCAATGCGCAGGGCCGGGCAGCTCGAGCCGAAGAGGAGCTGC
GCAAGAAGCAGGCCTATGTGGAGAAAGTGGAGCGGCTGCAGCAGGCGCTC
GGGCAGCTGCAGGCAGCCTGTGAGAAGCGGGAGCAGCTGGAGCTGCGTCT
GCGGACTCGCCTGGAGCAGGAACTCAAGGCCCTGCGTGCACAGCAGAGAC
AGGCAGGTGCCCCAGGTGGTAGCAGTGGCAGTGGTGGGTCTCCAGAGCTC
AGCGCCCTGCGACTGTCAGAACAACTGCGAGAGAAGGAGGAGCAGATCCT
GGCGCTGGAGGCCGACATGACCAAGTGGGAGCAGAAGTATTTGGAGGAAC
GTGCCATGAGGCAGTTTGCCATGGATGCGGCTGCCACGGCTGCTGCTCAG
CGTGACACCACTCTCATCCGACATTCCCCCCAGCCCTCACCCAGCAGCAG
CTTCAATGAGGGTCTGCTCACTGGTGGCCACAGGCATCAGGAGATGGAAA
GCAGGTTAAAGGTGCTCCATGCCCAGATCCTGGAGAAGGATGCAGTGATC
AAGGTCCTTCAGCAGCGCTCCAGGAGAGACCCTGGCAAGGCCATCCAGGG
CTCCCTGCGGCCTGCCAAGTCGGTGCCATCTGTTTTCGCGGCTGCGGCAG
CAGGAACCCAGGGCTGGCAAGGGCTCTCTTCTAGTGAGCGACAAACAGCA
GACGCCCCTGCTCGGCTGACTACAGACAGAGCACCCACAGAGGAGCCAGT
GGTCACAGCTCCCCCTGCTGCCCATGCCAAACACGGGAGCAGAGATGGGA
GCACCCAGACTGAGGGCCCCCCAGACAGCACCTCCACCTGCCTGCCACCG
GAGCCTGACAGCCTTCTGGGGTGCAGCAGTAGCCAGAGAGCAGCCTCTCT
GGACTCTGTAGCTACATCCAGAGTCCAGGACTTGTCAGACATGGTGGAGA
TACTGATCTGAAGGAGGTGGTGCTTCAGGACTCTGAGCCATTCTCTCCCC
TCCTCTGCCCTGTGCCACTCTCAGCCATTTCAGCAGCCCCGTCAACCGCT
GCTCCGTCCCTTTCCCCAGCCAGACACTCATTCCCATTGACCATCTGGTC
CCAGGAGCTCAGGAGGAGGACCCCAGGGGAGAGGAGAGCTGTGAGAGCAC
CGGCACCCCCAGAAGACTCTGCTTCTTAGCCCACATTCCTCCGGGCCTTA
TGGAGAATGAGGATTCAGCCTTGACTTCTTGCCCAAGGCCTGCTACTGGG
GTAGCAACTGACAGCTCAGAAAGGAGCTGAGCTCCCTCTGCCCTGCCAGT
TGTCAGTCAGGCAGGGAGGGAGTGGCTGTGTTGGTTTGGGGAACTAATTT
CCACGGACGGCTGCCCGTGGACACCAGGTGGACTGGTTCACTAATCAAGT
CAGCCATATTGTTCTCTGGCTAAGTTTGGTTCCAGCCAACGTCATCTGCT
CTTCAGTTCCTCACTGCCTTCTTGGGATACTAAGACTTGAATTTTTTGGG
GACTATTAAGGGTGTTAGTCTTGGAGAAGACACAGCCTCACCTTCTCACT
TGCTGTGGGTGAGGGGCCATTTAAGTGGACTGGGAGACAGTGCGCAGTTT
GTATATAATTCCCTTTCTTGTGGAACAGAAGACTGAGGCCTGCAGGTTCC
GATGTGTCTCCATGGGCTGTGCTCCCCTCTTCCTACTGTCAGTTTCTGAA
ACTTCTGACTGGCCTCCCAGTTATGCCTCCTCCTCAAGTTCCTGGCCCGT
GGATGTTAAAGCTGCTCGATTCCCAGGATCTCGGCTGCCTTTTCCTCTAT
CTTGAGCCCTATAAATGCCCACGGGACCCCCACCACCAGCCTCTTGAAGT
GGCTCCACAGCTCCTGTCCCTGGAACATCCTGTCAGTTTGGTCATAAACC
CTGAGCCAGATGAAATGAGCCACCGTGAACAGACATCTGCCATGCCCCCA
GGTGGGCTTCGGTGGCCCTACCCGGTACCAGTTCTTTCTGAGAAACTGGA
GATGTCTTGTTAGCATAAGTGTCTTCATTCCCACCTGGAGGGTTTGGGAG
AGGAGCAAAGCAGTTGAAAACTAGTTAATGAGCTACAAGAGTCAAATAGT
CCTCTGAATGGAGCCCCCATCACAAAACAGTGCCCAGGAGGCTGGCTCCT
CAAGCTACCCATGCCCAGCGCCCTAAAGCAGCACCAGATGCTTTGGAATT
GGGGTGAAACACCCACATGGCAGCCTGCTAGCAGCAGTGACTTTGACTTC
TGGTCTTAAAGAGTCCCTCACTTCAGCCCCAGGAGCTATTGGTGGGTTTT
AGCAGTTTTGTCTTTACCGTTTTTAGTTCTCCTTGATTCTTTGTTTTCTT
CCTTTATCGTTTTTAGGTTTGGTATGTGTTGTTTTATTTCCATGGTTCCT
CAAGTTTCCTTTTTAAACATTTGCATTTGCTGGACAATTGCAATTTTTTT
TAAAAAATTCCCCTACCCCTGTTTAAAGCTGAAAAATACATTTGGTTCAT
GTGCATTGTTTACAAAGCAAAAAGAAAAAAAAAAAAAAAAA

Ryk, or related to receptor tyrosine kinase, is an atypical member of the family of growth factor receptor protein tyrosine kinases. Ryk is involved in stimulating Wnt signaling pathways, such as regulation of axon pathfinding. The cDNA sequence of Ryk in Mus musculus is provided in GenBank Accession No. BC032275.1 (GI:21595526), incorporated herein by reference. The amino acid sequence of Ryk in Mus musculus is provided in GenBank Accession No. AAH32275.1 (GI:21595527), incorporated herein by reference. The cDNA sequence of Ryk in Homo sapiens is provided in GenBank Accession No. NM_001005861 (XM_001130617), incorporated herein by reference. The amino acid sequence of Gm5627 in Homo sapiens is provided in GenBank Accession No. EAW79155.1 (GI:119599561), incorporated herein by reference.

An exemplary DNA sequence of the Ryk gene in Mus musculus strain C57BL/6J chromosome 9, GRCm38.p1 C57BL/6J is provided below.

NC_000075.6 (GI: 372099101: 102834920-102908307):
(SEQ ID NO: 6)
CCCGTTCCCGGGCGGCGGGTCTGAAGCGGCGGCGCGATGAGCTGGCCGGCCGCCCCCGGCTCGGGGCTGT
GAGGCGCTCGGGGCCGGGGTGCGCGGCGGCGCGGCGGGCGGCGGACGCTCCTGCTCGGCGGCGGCCATGC
GCGCGGGCCGGGGCGGCGTCCCGGGGAGCGGCGGCCTGAGGGCCCCGCCGCCGCCGCTGCTGCTGCTGCT
GCTGGCGATGCTGCCCGCCGCCGCCCCGCGGTCCCCGGCCCTGGCCGCCGCTCCTGCGGGACCCAGCGTG
AGCCTCTACCTGAGCGAGGACGAGGTGCGCCGGCTGCTTGGTGAGTGGCGCGGCTCCCGGGGCCTCGTGA
GCCCGCGGTGCTGCCGCTGCGCCGCCCGCGCTCTCTCTCTCTCTCTCCCTCTCTGCAGCCTCCCTGCGCG
TGTCACTCTAGGCCGCGGAGCTCCGGAGCCTCGGAGTCGGCGCTCGGGACACGGGTGGCCGCTCCTTAGC
CTGCTGTGGGTGGCCCACAGGCCACGCCGCCGCCGTGCGCTTGCTTTCTGGTTAGCGAGTGGGGGGCAAG
GTGAGCCGGGCGCCCTGAGTTGTGTGGCATCCTGCACACCTTCGGGCCGACCGGATCCCAAGCATCGGGG
TTTGCCTTGGGTTCCTTGAGTGTGACCTGATGCTGCCGTTCCAGGCCACTTGCCTACTCTGCAGACCCTG
AAGCGAGAGGAGAACTTGGCTTTGGGGTCCGTCGGCGGTGGTTCCGTTTACCTGTTTAGCCCGTTGTTTG
GGGAGTCCGACTCTGCCCTCCGCTTGGAACTCTGTGCATTCTGAAAATCAGCCCTTGAAGGAAGGCCTGG
CCAGCCATCAAAAGGAAGTCTCTGATAGTTAGGGATACCCAGCTTTTGCCGTCTTTTGTGCATTAAGGCA
GTCCCTTCACTTTTTATCGGGGTGTTTTAGAGTGAAGTGTTGGAACTGGAAAGGTCTCCCCCTAAATATC
CATTAAGTGTCACTTTTTTAGTGAGCGAGTATGCCTCCCCCAAGTGTGTGTGTGTGTGTGTGTGGTGTGC
GCGCGCGTGTGTATGCGTATGTGTGTATTAGAGGAGGTTTTTTTGTTTGTTTGTTTGTTTGTTTGTTTTG
TTTTGTTTTTCCTTTCCAGACAGGGACTTTATGTAGCCTGGGCTGTTTTGGAATCCACTATGTAGACCAG
GTTGGCCTCAAGCTCACAGAGAGCCTTCTGCCTGTTAGAAGAGTTTCTTAGGGTCTGTTGAGAGAGTTTA
GCCTCGGAATGAAAACTGCTTTTAGCAGACTGTTTAGACATAGTTGTATGTGTAGGTTTATCGGCTTTAA
CTAAAGAACTGTGTTTTCCTAACATCCTAATTTAACCTTCCTTATAGTCTAGTTGAATTAGCTGAATTTA
GTCTAGCAGAATTGAAATGGCATCTGAGTTACGCCTGGAGCATGGGAGAAAGGCTTGGGAAAAAATAACG
ATTTGTAGTGCGGCTCCTAGGCGTGCTTTTGTAAGGGTTGTTTTTGTTGTTGTTAAAAGAACTTACGTAA
TTGTGACTTTATTGACATGTATTTCCTCTCCTGCCTTAGCCAGGATGGAAGTAGGGAGACAGAGATATCT
GGCAGCAGACAGAGACGAGTACATGTGCCTACCGTTGCGTGTTAGAGTTTTGCTGTTACTTTTATTTATT
TTGGTGTGTGTGTACCACGATGTGTTCTGTGGAGGTCACAGGACACCTTGAAGGTGTGGGCTGTCTTTCC
ACTATTAATAGGGGGTCCCAGGGCTCAAACTCATGTCATCTGACTGGACGACAAGTGCTTTTACCTGCTG
AGCCATCTAGCTGGGGCTTGATATGGACTTAAACATGTTGTTTCAGTTTCTTGTTACCCAGTGGGGCAAT
TATGGGAGATGAGAAAAGTAAGGCTTTCAGTAACTTGCTGAAGGTAACATGGTTTATTTCATATATTACA
TATATAATATATACCCACACAAGACAGTGTTTCCTGGCTGCAGACTAAGTTATTGAAGGGCATGTGCATT
TAGAAGTTTGATTATGGCATGTTAAAAAAGAGACTGTTGTTCATTAGTACGTGAAGTGATACCTTTGTAG
CCTGGAACTGATTTTCTTAGGACAGTAGCTGAGGGGGCACCTGATGGATGAGCACCTGGTCCAGTAGTGT
GTGAAATAGAGTGGGGAGCATGGCTCATTAATGCTCCCATCCACCCTTAGGTATTTCAGTTCCATGCAAG
CTGAGTTGTCCTGTTACAATGTGGTCTCTAGATAGTCCTCTTGCAGAACCAGGGCCTCTTTCTGTCCCTT
TACGTTTCCCTCAGCTGTGCTGCTGTTGAAGGATTCCCATCTGGCTTTGTCTGGGTGGCAAAGTGCAAGT
ACTGGGACGTTTGCTGGGCCTCCTGAGTGTGGCTGTGATGGTGTGTACGTGCGAGACTGTTGGAGATAGT
GCTTTCCACCACGTTTTCCTTTGCGTAAAGGGTTAAGTGACTTCCAAAAGCTCAGGCAAGCTAGTGTCAG
TTTTACAGTGTTGCTGAGACCCAGGGTTCAGAATGCCAGGTGAGAACTTGGAAGTTGGAAGTGACAACTC
TGTGGAGCTGGCTGTACTTGATAGGAGAAGTGCTTTTAATCCAGACATTAGCTGCACATAATAGAAATCA
ATTGGATAATAGCAAAAAGGCCTTGAACCAGAAGCCATCAGCACCCTGCCTTTGGGCTTTTTGCCCTCCA
GGTCCCACTGCCCCAGTGGGCGTAGTTCACACAACTGTGGTGGAGTTTGTCTTTCCCTTGTTGTCTTTGG
AATTTCTAGACTAGTGTGGACTGAACATTTGTCTTCTGGGGTCCTCCTTGTTCATCAGACTCCCCAGGGC
AGGTGGGTTTTTTTTTTTTTTAATTTTAAAAAACATCCCAATCACAAAGGAACTCACACAATATCTACTC
ACTGATAAGTGGATATTAGCCCAGAAACTTAGGATACCCAAGATATAAGATACAATTTGCTAAACACATG
AAACTCAAGAAGAACGAAGACCAAAGTGTGGACACTTTGCCCCTTCTTAGAATTGGGAACAAAACACCCA
TGGAAGGAGTTACAGAGACAAAATTTGGAGCTGTGACGAAAGGATGAACCATCTAGTGATTGCCATATCC
AGGGATCCATCCCATAATCAGCTTCCAAACGCTGACACCATTGCATACACTAGCAAGATTTTGCTGAAAG
GACCCAGTTGTCTCTTGTGAGACTATGCCGGGGCCTAGCAAACACAGAAGTGGATGCTCACAGTCAGCTA
TTGGATGGATCACAGGGCCCCCAATGGAGGAGCTAGAGAAAGTACCCAAGGAGCTAAAGGGAACTGCAAC
CCTATAGGTGGAACAACATTATGAACTAACCAGTACCCCGGAGCTCTTGTCTCTAGCTGCGTACGTATCA
AAAGATGGCCTAGTCGGCCATCACTGGAAAGAGAGGCCCATTGGACTTGCAAACTTTATATGCCCCAGTA
CAGGGGAACGCCAGGGCCAAAAAGGGGGAGTGGGTGGGTAGGGGAGTGGGGGTGGGTGGGTATGGGGGAC
TTTTGGGATGGCATTGGAAATGTAAATGAGGAAAATACCTAATTAAAAAAAAACATTTATTTTTATTTCA
TGGGCATTGGAGTTTTGCCTGTATGAGGGTATAGCATCCCCTGGAACTCGAGGTACAGACAGTTGCGAGT
TGCCATGTGGGTTCTGGGTATTGAACGTGGGTCCTCTGGAAGAGCTGCCAGCACTCTTAGCCACAGAGCC
ATCTTTAATCCTCCAGTGCATGTTTAAACCAGCAGTCAGTTGCCACAGTGGTCTGGTCTGTGCCCTCAGA
TGGTCAGTATCGATGCTATAGCGTACAAATACATTGCCCTTTTCTGAGTTCGATATGAACTGGTAGACAC
CCCCTCCCACACTCACCCCCTTTGTTCGGGTTTTCCCTTTGGATCTTAAACTTTTTTTTGATTAGCTTGC
TTTGTGTCTGTACATATATTCTCTCGGGTACCCCAAGAAGCACGCTGAGTGCCTTTTGATCCCCAGTCCC
CTTCAGGCAGCACTTTAAGACATCTGTTAGTTCCATTGGATTCTTCTCAGTGGTACCTTCCTTGATTTAG
TGTCATCCATCCCTTGCTTCAGGCTGGTCAGTAGCACTTTATACCTCTCTGTCCTCTCTTGGTCTCCTGT
CCTCCTTTTTCTTTGCTGGTTGTGTGTATCCTCAGGGGAACCCTAAGGATGGGGTTCATGGGGAAGAGAT
ATTATTTGAGGTCCTTTATGTCTGAAAATTTTCATTTTACCCTCACACTTTGTTGATGGTCTGGATGACT
TTAGAATATTAGGTGGAACATAATTTTTGTTCACAATTTGGAGGGTTATTTTCTCTTGTAAGGATTTCAG
ATAAAGAGCCCAAAGCCATGTCTTTCTAGCTCCTTCCCCCACCCCCTTCACAGTTTTTAGAATTCCGGAG
TGGTTTACTCTGGAATTACTCTGGTATTCTGAAGTTTGTTAATTGGGTTGCAAAGCCTTCAGTTTTGGGG
AGTGTTTGTTTGCTTTTTAAATAATATCCTATGCCTTATTTCTTCCTATGTGGGTTTTTTTTTTTCTTTC
TTTCCTGAGATGGCTGTCGTGTGTCTGTTGATTCTCCTTAAGTTGAACTTCATGCTTCTTGTCTTACCTT
TATTGCTTTGTATTGAAACCAGTGGACAGAGAGTTCTCCTCAATTTTGTCTTACCAGCTTTCATTTTAAA
AATGTTACTGATGTTTGTAAGTTTTAGAATCTCTTTTTTTTTCTGTATTTGATTGCATCTTGTGAATGTT
TTATCTTAACTTTTTTATTATTGTTGTTGTTGTTGTTGCCAGAATTAAACAGAATTCTTCTGCTTGTGGT
TCTGGCTGTCTGATCCTTGGCTTTGTGTAGTGAGATGCTAATATTGTTAGGAGGGAGAGGATATGCTCGT
GTGCTGACCATCCTTGCTGTCAGCCAGAGTGCAGGTGGGCATTTCGCTGGAAGCCTGTCCCGAACACCAG
TACAGAATTTTGTATCTAATTAGGGCTCTCTGCTCAGTGTCTGTAAAGAAGCCCCAGGAAACTTCCTGCC
TCTGGAATGCCGGGTGATAAATCAGCTGCCAGGCCTTGGGTTACCTGGTTGGGGAGGAATGGAAGTTTTC
ACCCTCAGGCTGTAGACTTTGACTTCTGGTTATAATTCCCTGCCCTTATGACTCAATTTCCCTAGACCTG
GGGTCTCCTACTGAACCTGGGGACCTGCTGCTGTGGATATTTGCACAGTGGCTCCCTCCCTGTGCAGCCT
GAGCTTCAGGGTGACCTTTGGTACCTCCCAGGTACCTCTAGGGCTGAGCCTCTTAAGGGGCTTCTGATCG
CTCCTCTACCTGCTTTCCATCATGCAGAAGTCTGATAAAGTAGATGAGTTTTTTCTTCCTTCTTGGCTTG
ATAATTCAGTTCCCCTACCATCCAAAGGCTTTGGTCATTCTGAGCTGTGTCATTTGGTAGTTGTTTTGTA
AGGGCAGTGGTGGTCAGTAGTGGTGGACAGAGTGCTCATGATGTGTGGCAGAAGTTTCCCCGTGTCAGAT
ACATAGTCCTCGAAGGCGGGTGCTGACACTTCCACCCTGTACCCAAGCAGGCTGAAACAAGATTCCATCA
ACTGAGGCCCTTAGAAGCAACAGAAATGTCTTTTTCACAGTTCTGGATGCTGGGAAAGCCAAAATCAAAG
TGCTGATGGCTTGGTTTCTGGTCAGGGCCTGTTTTCTGGTTCCTAGATGTATCCGTTCTCACACAGTGGG
AGGGTGAAATGAGCTCTGTGGTCTTCATTATCAGGGCACTGATCCTCCTTGTGTCTTTAAGACCTAATCC
TCTCCCAAAACCCTGTTGTTTATCTATCTGCCCGCCATGGCGGTTAGAATTGTAACATGGGTTCTGGGGG
AATACAGAGGTTTTTACCTTAGCACTGAGGAAGCAGACTTGGAGGGGGTCCTTGGTGTGTGCCTGCTGCA
TGCAGTCGTCCTGTGGAAGTCCGCCTATGTTCTGAACCGTGTCTGTGTGGTTTCCCGTGTCACCAGGCCC
TGCTTCACTGCTCTCATTACTGTCCTTAGCAGACTACAGTACTGGCTACTGTGTCCATATGTGAAGGGTG
GGATCTTGATTCAGCGTGTTGTGGACATCGAGTAGAAGGCCAGGCCTTGACTCAGTTGTGCCTGAAAACT
GGATCCCTGCTGTTTCCAGCAGCCCTGGAGCCGCTGCAGTTAAGCCAGAACTCAAACAGTGCTGGTTTGT
GGACCTGGTTACTGTCAGTGTCCTCCTAGGTTCACTAACGTAGCCCTGTGCGCTGCAACTGCTGGAGCAG
TGCCAGAGGTTTGGGAATAAGATGCCGGTGGCTGGGAAGGCGTGGTGACAGTGATGGCCGTGATAGGGAG
CTGTGGTCCATGGCGGTGAAGACAGCTTCGGAGAGAAGGAAGCAGTTAAGCATTTCCTCTGGCTAGCTTG
CTTCTCCTTTCAAACTAAATTGCTTCTGGGAAAGCCATGCTTGCTCACTCACAGCTTATTTAAAGACAGT
TCACACTTGTTTAGGCACTTGCTTCTCAGAGCAGTGTAGGGAAGGAAAATTGAAGGATTATATATATGGA
GATAATGACAAAAAGTGATTAGATTCCTTTCCTTAATGAAGTGTGTGTGTGTGTGCATGTGTGTATGATT
GTCCCATGTAGGACTGTAGCTCTGGGATAAGATTATTGGAGGAGTGGTCACTTGATTGTGATTGATGGGG
TGATAGGCAGGGTTATTTTTCATTTTTCACTATGGTTGTTGGGAGGGTGTTTTTGTTTGTTTTTTGTTTT
TGTTTTTATTTGTAGGAATGGTTACTTTTGAGAGAGGAAAGAGAGTCTTGGAGAGTTTGGGAAGGGATCC
CAGGAAAGGAAACATTTACACCTCATCTCCAGGTTGAGCTAGACAGGCAGACAGAAGAGAGAAGCACTTA
GGGCCAGGCAGAGAACATGGCATGGAGTGTGGGGGCAGACACTGCAGAACTCTGACATGGCTGGGATGAG
AGCCATGAGCCAAGGAGAGGAGACAGAGAAGACAGCTGGGAGATCAGCGGATTGGAGCTGTTCCCCTGGG
AATGGAAAGCCACTGGAGTAGTTAGGCAGCACTCCAGAGGTGGAGGCAGGGGGGATCAGGACTTCCTGGC
TAGCTTTGTATATGTGAGACTCTGATTTACTCCCCTCCCCCAAACAAAATAACAACAAAACCCAGCCATC
AAAATACAGTACCCCACAACAAATAAAGCAGAGATAGGAAAGGAGTATGCAGTCGTAGTGGGTTGTAGAA
AAGATAGTGTGTGGCTCCTTGTCTATAGTTAAGACACCTGTCGGGGAACATGGGTTGGGGAGCAGGTGAA
GAGGTGGGGTGGATTTATCAGGAATGAGCAATGAGTTGGGAGGTTTACCCCTCTGAGACCTCAGGGCTAA
GGAGAAGCTATTCTCTAGAGTCCACTTTTTTAGATTTGGATGTCTGAGCCAAAGGACCCTGTCTCAGTTG
AGATGAGCCAAGTGTTCTCCCCAGCTAAGCTCCTCTCGTTCCTGCAGAGTCTCAGGCCAAACGCTTGGCT
GGACAGGGGTGGTGTCCTCCCTGCTGGTGGCACTGGTATAACCTGCTAGGGGTGGCTTTGGCAGGAGTGG
GTTAGCATCCTGAAGTGAACCCTTAACCCTAGGCGTTGGGTTGGGGTTGGTGGTATTGGCACAGCTGAAT
CCGAGCCCATGTGTTCTTAATTGGGAGGCCGGGGCTGAGACATTGTGCAATTGTGGGTTTGAGTTTCTTT
CTCCCTGCACATATGGGGGAAAATTCTGTCCAATTATGGCTATACTTGTCCCATGTGAGTCACAGCCTGA
GTTAATGAGCGAGTTCTGAAGGTGCTGCCTCAGTGCTGCTCGGGTTTTAAGTCCACTCCAAGCGTAAGTG
TGTGTGTGTGGGGGGGGGGCACGTTTCTCAAACACTCAGAACTGAGAACCTCCCCAGCTTTGCTTTGGGG
TTTTGATGCTGTACATCAACTGTCCTTTTAGATCTTGCCAGCCTGAGAGATAAGAAAAACCTGCCCTTGA
GACCAAACTATTAAGTTACTCTTAAACTACAGCAAAAGTACCGTGCTTTCTGTGTGTGTCATCCTTGTGG
ATGTATGTGTGTTCTAATCTGGATGTTACTAAGCAACATTCCATTGTGTAGGGCCTCCTAAAACGTTATG
TGCTAAAACTAATGTGTGTGTGTGTGGGGGGGGGGCTGAGATGTAAACGCTTACTGAAGGGTCTGTTTTG
CTGTCCCCAGCAATGACAATGCCGAGTGACTGTCATCCTTACATTTTAAGTTGTAATAGAATGTGTATAG
TTCTTAACTTGGGAGATGCCCATAATCTAGAGAGAACTAACACATCTTATGCACACCAGCTATTTCACAC
ATGTTTGTCAGCAGCATCTGGCACATTGTTTACTTTTTAAATAGATAAAACCAAGATTCTGGAAAAATAC
ACAATTTGCTGGAGGTCTCAGATGCATGCAGTTACGAAGCTCTGAAGTGCACGCTTTCTACTCTGTTTCT
GGGAAGGGTAATAGTCTACCCGGCTCCCATTGAACTCACCCGGGGAGCTGCAACAACATCCAGAGTCCTG
GCTATACCCTTACCAAGTGGTGTAGTGATCTGAACTTCTGATCCTCCTGCCTTCCCCTCCCAAGCTCTGG
AATTCCAAGTGTTTACAACTCCTGCTTCACGTGGTGCTGGAGATCAAACCAGGGTTCCGTGAATGCCGTA
CCCCAGAGCCAGGTCCTCAGCCCTTGTAGTTGATCTGAAGTGATGAGTCAGCCCTTTGACTTCTGAATGC
GTCACTGACTGTAAGATGCACAGCTTGTAAATTTGCAGCTGACTTAGTGGGAGTTCTAGTTAACAGACTG
CTTTCCCCCATACCTCAGATTAAAGTGACATGCCCTGTTATCAAGGGTGACACTGTAATAACATCATCAG
CCTAGCATTTCAAAAAGTCCAGGTGCTGTCCTGGGAGTGCCAGTGGTTTTCTCCTTCCTGGTAAAGGGGG
TCTCTATAGTAAGATTGCATACTTTGCTAGAAAGTCCTTAGGCCTGGGGCCTCAGAGAACTCCTGCTGAA
GTTCATGGGTATCTCTGAGTCTTTTGGATTTTTGAGACAGGGTTTTTCTGTTTAGCCCTGGCTGTCCTAG
AATTTGCTTTGTAGACCAGGCTGGCCTTGAACTCACAGGGATACACCTGCCTCTGCCTCCCAAGTGCTGT
GATTAAAGTGTGTGCCAGCACTGCCCAGCTGTATTCTAATTTCTTACAAGAGTTAAAACAGTTATGATGG
GGTTGGAGAGATGGCTCAGCAGTTGAGAGCACTGGTTTCTGCAGAGGACTGAGGTTTGACTCCTGGCAGC
TGCATGACAGCTCACAACTGTCCATAACTAGATCCAGGGGATCCAGTGCACTCTTCTGACCTTGGGTACC
AGACACACATGTGATGCTCAGATAAGCCTGTGAGCAAAATACTTATACACATAAAATAAAATAAACAGAA
AATAAAATTTAAATAAAAAGTTGTCTTTAAGAATTCATTCTAGAGCTGGAAAAATGGCTCTGAGGATAAA
TATGTATTGTATAAGGCTGGAGACCTCAGTTTGATCACCAGGACCCACCCGAGAGGAATGAATGACATCA
ACAAGTTGTCCTCACACCTGAGCCCTTCCCATAAACAAACAAACAAGTGAATAAATGTATGTATGCAATT
AAAAAGTTCACTCTGCTATAAGCTTTTATGGGCTTTGACAAGTGCATAATGTCCTCTGTGTACTTCATCA
TATAGTATATAAAGAATATATCAGATATACAGAGTAGTTTTAATAAAAAAAATCATAGGAGGAGGCATCT
GCATTTATAATAACAACAAAAACCCAACCAGTCAACCAAACAAAAAGCCAAGAGAGCCGGGAAAGAAAGT
TTCCAGTATTCATTTTTCTGAAGTCCTGCTGTTCCTCTAACCCAGTGCACTTTTTCTTATCTCTTACATT
GTAGTTCTCATTTCAAGAAATTAGACTTTATAGTCTTTTTTTTACTATCTGCATTACTTATTCTTTGTTT
CTGCGGACATTTTTGGCCACGTGGAATACGGTTACATAGTTATTGCTAGCACACCAGTGCCTGTTGGTGA
GTTTTAGTTGTTATCTAGTGCTCTGTACATTCTTTCCTTTAAAAAAAATATGTGTGTCTATAAATATATG
TTTGGATGGCAGATACTGTGAACATTTCTGGGTGCTGAGTGTGTGTGTGTATGTGTGTGTGTGTGTGTGT
GTGTGTGTGTGTGTGTGTGTTGTGTGTTTTGTGTGTGTGTGTGTGTGAAATTTTTTGAGACAGGATCTAT
TGCTTGCCCTAGAATTCAATGATGAGGCTCAGCTGGGTGGCCGTCCAGGAAGCTCCAGGGAACCTCCTGT
CCCCATCTCCTCAGGGCTGGAATTACAAGTGTATGCCATTGCACCCAGCTTTTTTGTGGGTGCTGGGATC
TAAACTCTGGCTCTCTCTTGTAGCAAGCATGTTAGGAGCTGAGCACCTCCTCAGCCTGCCTACCTGCCTT
TCTTTCTTTGCCTTCTTTTCTGTTTTTGCTTTTGTTTGTTGGTTTCTTTGTTTTAGACAGACACTTGTTC
TGAAATTCAGGCTAGCCTCCTGCTTCATTCTTCCCACATGCTGGAATTGTAGGAGTGTACCAGGATGCAT
GACAGCTGAACATTTATGAATTCTTTTAAATATCCTTGGATTTTGTTGTGGGATGCAGTTAAATGACTTG
GAAACAGTTTGAGCCTTCTGGGTGTTACTTATAAGGCTTGTTGTCAGGCTCATTGTTCCCCACAGGTGAG
TTAAGACCCTTCTGCACTTATCCCACTAAACCGTGAAGGGTGAGCCTCCGGTCTGGCTGGTGGGAGGATC
CAGATTCTGAGTATTGGGCACTGTTCCCTTTTACCCTCTCTCTGCCCCCTTCTGGCTTCCACATTTGCAT
GTCGAGTCACAACTCAGATGGGCTCTGGAGCTCAACACTCTGGACCTTGGACTTTCCACTTTGTTTAGCT
CTCTGTGCACCATACCTCTAGAATCTTTTTCTAGATTCGCACATGCCACAGCATTCATGGGGAGGTCAAA
GGACAGCTTTGAGGAGCCAGTTCTCTCTGCGCTGTATTGAGCTTTGGGCATCAGGCTCAGTCCTGAGACT
CTGTGGTGAGCGGTCACACCTGCTGAGCCATCTCTCTTGACTTCAGCATCCTTCTCTGCACTGTAACTGT
AGCACTTACTACTTGTGGTCTTTGTTCTAGGGATGGGTCACTGTTGCCTCCTGATGAGTCCTTTAAATTA
ATGAATCAGTTGAGTCCAGTCTTGATTATATCAGATGAGAGAATCAGTCAATCTTCCAATATTCTCATCA
AATGCTCGTTGTTCATGTCTTCCCTAAGGTTAATTCCTTAATGATCAATTAGAGGAAAAAGTAGCCCTGC
TGAAAGAAGCAGTTGTGTAAGGCTTGGATCTGTTTGTGCAAGCACGCTTGTGAGCATATGGTAGGTGGCA
AGTGTGGGAGAGTGTGGGAGCCCATATGTTTGCATGTTAGTCGCATCCCTTCCTTCCACTTTAAGTGGTT
AGGCATGTGTAGCGGCAAATCTTGATGGTCAGGTTAACACACCTGGAAAGAGGGACCCTCAATTCAAGAA
TTGCTTCCATCAGCTCGGCCTGTGGACAAATTTAGGGGGGCATTTTCTTGATTAGTTACTGATGTGGGAA
GGCCTACCACACTGTGTTCTGTGTCACCTCTGGGCATGTGGGTCTGGGCTGCATAGGAAAACTATCTGAG
CTGGGTAGTGGTGGCACACACCTTTATCCCAGCGCTCAGGAGGTAGTGGCAGATGAATCTCTGAGTTCAA
GGACAGACTGGTCTACAAAGTGAATTCCAGGACATCCAGGACTACAGAGAGAAACCTTGTCTCAAAGTAC
CCCTCCCTGCCAAAAAAAGTTATTTAAGCATGAGCTAGTGAGTGGGCCAGTACGTGTAAGAGAGTTTCTC
TGTTTCTCAGTGGGTTCTGCTTCATTTCCTGCCTGGACTCCCTCATTGATTGGCTGTGACCTGCCGTCTT
CTTTCCCGAGTTGCTTTGGGTTATAGTTGTTTATCAGACCTACAGAAAAGCAAGCTGTAACTAATGATCT
GCCTATGTAAGATGAGTTGCTCTTCGCACATGTGCTATAATGCATTTGAATATGCTGAATGAGCCTCACA
ACAGCCTATGTTAGTCACTGCTGGCGATTTCTGTATCTTTGCATTAAAAACAAATACCCTAAACATTTTG
AGCTGTTTCATTTCTGGTTGGGCTCTGCTGAGGAAGGTAGAATGGCTCATCTGTTTGGTTCTGAGGAAGG
ACTGGGAGTAAGCTGCAGGACACAGGGAAGGAGCCTGCTGATCCTCCCCAGGAAGAGCCAGATACCTGTA
GCCCCGAGCCCCTTTCCTCACATTTAACTGTGTGAGGAAACACAATTCCTGGCCTAAGACAATAGGCCAC
TTAAGCACAACGCACTGGAGGCCTCAAGTCACCGGGTCATTTAGATATTTGGTTGGTTGCCTTTCGTAAC
AGTATTCTTTTACAAGAGTAATATGAGCTTATTTTAGGAGCATGGGGTATCTTTGGTGACTAAAACAGAT
AAAGGACCTGGCCTTGTGGAACTAACTTTTGACAGAGACCTATGTGATTTGCCTTTGAAATTAGCTTTGG
GATTGGATTCCTGAGATAGTAGCACCCTGCTGCCCGTCACTGGCAGGGAGAAGAAGCGATGCTTGTGAGT
TCTCTGTGTTTTATGATAACAGCCTACATTATTACCAGGCAAGTCTGAGTCAGTCCAGCCTGAGGTAAAG
TGTGCTCCTGTTAGCAGTGGAGGTCAAAGAAGTTGGGCAGTGCTGCCATGAAGCCCTGGGTATGTGGCTC
CATTTCTGTGGCTGTGTCAGTGGTACAGGTATAAACTGGTCATATCACTTTCTTCTTCTAGGTCAGGGAA
CACACCAAGCTATTGTCTAGAGTCTCTGTCTTACCAGATGCTGGGGATTAGGGCTCTGTGGCGGGGAAGT
GGTGTTTTATGTTTTAATGCTGAAGCTTGTGTATCCTTGAGAGGGAGTTGGGAGGTGCCTTCTACACTAT
TGGCTATTGTCCAGATCATATGCAATCACAGTTTTTAGGGGAATCTTCTTACGCATTTCTCTTTGACCTA
TATTTTCCTTGGGAGTGAAGTGTTTGGTTGTTTTAATTTAAAGTGTGTTTGCTACATTTGCTATAGTGTT
TGCTGAGGTGGGGAAGGTGACAGCACATCTATTTGAAACCACGCCTGCATAACTTCTTCCAGAGCACTAG
CAGAGACAAACCAGAGCAGCACAGCTTTTGTGATGGATGGCTTGAGTGTCTGTGATCAAGCATCAGAGAA
TTACTATGGGTTTTGAGAAGACAGGTCTTGGTCTGAAGGAGGGAGCCTAAAATACAAGGAGCAGAGTGGG
GGAGTAATGGCCTTTTGTTGGTCAGAGCTGTCTTTCTCAGCACCCATGCTTGTCTTGGTTTTTCTGTATG
TAACTATGCATTGTTACCTCAGCTCTGCCAAGATGTTTGAGATAGAGACAGAGACAGAGACAGAGACAGA
GAGAGAGAGAGACGCATGAAGCAAACACTGTGTGGTGGAGTGCCTGAGAGTTGTTCCCAGTGTTCCTGCT
CCAGTAGCCACAAGGCTTGAAGAGACCTTTGTAGTGGTGGAAGAAGGCGTTCCTTTCCTGCCATCAAATG
TCGCAGTTCTCTGCTGAGGGCAGCCACGTTCTTCTGGGACCTAAACTCAGAAGCTCTGCAAAAGCAGCCT
CTGCAGCTCTGGCAGTCGCTGCTATGTGAAGTTAAATGTCGCTTCTCAATAAATTGTTTGGGTGTGCACG
GGCATAGCTAGATTTCGATGAACAAACTTATTTCAGTTTATTATTTTATATAAATTAGAAACATTAGAAG
TACGAGCTTTTTATTTTGTATCAGTAAGTGTACTTAGTGGTTATACATTGCTTACAAATAAACAAAAAAA
TAAATAAATTCCAAGAAAAAAACCACATTGATTACCTTTTTCTGATTAGACCTAGAAGATTGTCTTGACT
TGAAATTTTTTAAAAGTTTTGATTGGTACGTGGGAGCCATGGATGTATCATAGAATACTTATGTTTTGGG
TGACATTTGGAAACTACTGCTGATGTCATAAAATTATAGAATTTTTAGTGGAGGAGGGGATTAACATCAT
CTGGTCTACCTCTTTTGTCTTATAGTTTTGAAAGCTGACTTATGAAGATGGCTTAGGAAGTGAGTGCCAA
AGGCCTCTCACCTACTTAGTCTCAGCCAGGAAGAGAGGTCTGCACGGTGAAAGCTGCTCTGTTTTCTTAT
TTTAACTGATGCCTGACAGGTTGTAAAATAACTTTTTTTTATCAACAGCCAGCTATTCATGTGTCTGCCA
CTGAATGCTACAATGCTACCCTAGGTAGAGGAGACTGCTGGTCCTGTGAGCAGCTCTCAGACATATGTCC
TGTCAAGTGCACTCTTAAAAGGAGGCTCTTCAGTGACATGCAACTTACCCACAGGATGGCAGCATTGGAA
TCCGTAGTTAACTTTCAAAGTCATTTGGAAAAATTTTAATTAGTGGATTATTAATTTGGGACTTAAAATT
GTATAGTGAAGAACTTATTTTTCTTGATAGGGTCATGAGAATTTTGCAAGAGTAACAAATTTTATATCAT
CAAATATGGGTTCTTTAACATCAAATTATGAACAAATGTTATCCTTTCTTAGAGAGAGACACACGCTACT
TCCCTACTTTGCTGGAATTGGCAGCAAGAAGCTTAAGTCGAGCTGATGAAATTGAGCAGCTGAAGTGCTT
ATCCTGAGTCAGAATGGCAGTATTCTGTGTGTTCTGATGACAAAATGCAACCTAGTCAGTCCATGGCCTG
TGGCCTTTGCCCTCTCTCCCTCCTCATAGCCCTTTCCACTCTCTGCCTGTTCTCCTTTCCCTGTTTTGTT
CCCTTCTCTTTGGCGCTCCCTCTTTATATGGTAGCGTTTCAGTTCTTGAGTCTCCATGGTGCTGCTTCTT
TCAAAAATCAGTCTGTCTGTCTGTCTTATCATCTATCCATCTATCATCCATCTATATCTTCTATCTATAT
CATCCATCTATATCTGTCTATTATCTATCATCCATCTCTCTCTATCTAATCAATCAATCAATCTATCTAT
CTATCTATCTATCTATCTATCTATCATCTATCTGTTTATCATCCATCCATGTCATCCATCTACCTACCTA
CATACATACCTACCTATTACCTATCTCTCTAGGGTGTGTATGTGTGTGTGTGTGTGTGTGCGCGCTATGC
AAACAGAAGGTTTGAGGACAGCCTGCAGGAATGGGTTTCTCCCTGTTTGGCTTCTGGGGTCATCTTTGGT
GGCAGGCACCTTTACCTGTTGAACATCTCACTGACCTGTTCTGTATCTTTACAAGTGGATTCACACACTA
TGGGATATTTTTTCTGACCATCTTCATGTAGCACAATGTTATGGAGGTTCCCATACATCTTTTCATTCTA
TTTTCATACCATAATTTACTTTTTATCTGTTCAGGGACCTTCAAGTTACTTACATATTTTGGCTGTTATG
AATAATGGTGCTGAGAATATGCAAGTACCCGACTGGATACATGTTCAAGTTAGGTACATAGAATAGAATT
GAGGGTTATCTGGTAACTGTCTTAACTTTTTAAGGCACTGCTAGGGGCATTTTCAGTGGCTGACTGTTTT
GTATTCCTACCAAGACTGCAGGGGTCTTGTTTCTCTGCACGCCAGCTCTTGCTATTTTCTGCTTCTTTGG
TTGTGGTTATTCTAGGCAGTATGAAGTACCTCATTTTTATTTTGATTCTGTTTTCCTGGTATCGAATGAT
GTGGAGCATCTTTACATGTAGTTGTTTATATGTCTGTCTTCTTTGGAGAAATGGGTGTTCAAGTCTGTTG
TCCACTTTTAGATTGGACTGGTGGTGGTTGCTGAATTGTAAGAATTGTTTATATATCCAGGATTCACATA
CTTGCCAGGTGTGATTTGTGGGGCCATCCTCCAGTCTGTGTGTTCTTGTCTTTGTAATAGTAGGTTGTTT
CGTTGTTTGTTTGTTTGTTTGTTTGTTTGTTTGTTTCTGGTTTCTGCATAGAATTAAAACTTTTTTAAAT
ATGAATTTTGGCTTATCTACTCTTTCTTTCCTTCTTTCTTTTTTGTTTTGTTTTGTTTGAGACAGGGTAT
CTTTATGTATCCGTGGCTGTCCTGGAACTGGTTCTGTAGACCAGGCTGGCCTTGAACTCAGAGATCCACC
TTCCTCTGCCTCCCTAGAACTGGGACTGAGGGCGTGTGCCACCATCACCTGGCTAGTTTATCTTCCTTTT
CCATCCCTTGCTTGGGCTTTTGCTATCCTATTGTAAGAACTCGCCACCAAGGTTATGAAGATATATCTTT
AATATTTTTCCAAGAATTTTATGGGTTTTTTGTTTTGTTTTTCTTTTGTTTTTCAAGACAGGGTTTCTCT
GTATAGCCCTGGCTGTCCTGGAACTCACTTGGTAGGCCAGGCTGGCCTGGTTTTATGGTTTTGTCTTATG
TTTGGGACATCTATTTTAAACTAATCTTTGTCTGTGGTGTGAGGTAGGGACCCAGTTACATTCTTCTTAT
AGAATAACCATTGTCCTACCATTGTTTGTTGATGGTGGTTTTGGTACCCTTATCAAAACTTGAGTCTTCA
GTTTTGGGTGGTGGGGGTGGGAGGTGGCTTTAAGACAGAGTATTTCTGTATATTCTTGACTATCCTGGAA
CTCACTCTGTAGACCAGAGCTGGGCTCAAACTCACAGAGACCTGCCTGCTTTTGCCTCTGGAGTTCTGGG
ATTAAAGTTGTGTGCTAACTACCACTGCCCACTGCCTGGCAAGTCTTCAGAATTTTATAACTTTATTTCT
GGCATCAATTCTGTTCTGTTAGTTTACACAGCTGCCATTATGTTAGAATCATACTTTTTAAATTTTTCTG
AGATTATAATCATATCATTTCTCCCTTTCCCTCAACATTCCCATATATCCCTCATTCCTTGTTCAAATTT
ATGGTCTCTTGGTTTCTTTCTTTTTTGTTTGTTTGTTTGTTTTGTTTCATTTTTAATGAGAGAGAGAGAG
CGCCTAAATACATAAATACAACCTGCTTCGACTGTGATGTTATTTATCTGTGCTTCCAGGCCTCATGAGC
TTTCCCTTGTCTACGTTAGATGTCTGTTGTTGTTCTTGTTCAGTTCGTGTTTAAGCAGTCATGTTGGTGA
GACTTTATGGCTGTCACTTATGACATTCTCACGGCAAACTTCCTGTTCCTCTGATTCTTAGAATCTTTCC
TTTTCCTCTTCCACAATGATCCCTGAGCCATAGTTACAGGAGTTATGTTGTAGATGTATCTGTTGGGATC
GGGCGCCATAACTCTTCATTTTGCTTGGTTGTGGTTTGCTATAATGGTCTCCTTTCATTGTAAAACGGAG
AAGTTTCCTTGCAGAGGGGGAGGACTTACCTGTGGGTGTGGGGAAATATTTAGGATGTGGTTAGGGGTCA
TGATGATTTAGTAAAGTAGAAGACCCATGATTTTACTAGCTGTGGGTGGTTGGCTGGGTTTCCAGGACCA
GGCATGATTTCCCTCTTGTTGAGCAGGTCTTAAGTCCAATTAGAGAGTGCTGGTTACCGCCGAGGTACGT
GTGCCACTTCTGCACTGTCTTGATTTGCATAGCTTTGTAGTGAGTTTTGAAACTGTGAAATGCGAATCTT
CTTTTATGTGCTTTTAAAAGATTGTTTTGGCTATTTGGAGCAACTTTGCAGTTTCATATCCACCTATCTA
CTTTTCTATTTCTGTGTAAAAGGCAGGTGGAATTTTGGTAGAGAATGCACTGACATTGTACATTTGGGTT
ATATTGCCATTTGACTAACCCTGCTGTCTTCCCATCCGTAAGCATGCGATGTTTTTCTGTTTATTCGGTT
GTTTCAGTACTTTGTGTTTTTTAACTTAGATTTTCATTTCTTTGGTTACATTGCTTCCTAAGTATTTTAG
TCTTTGCGATGCAGCTATGAATGGAATTGGTTTCTTAATTTCCCTTTGGAATTGTTCTTTGAGGAACTGA
CTCTTATGCTTTCTATCTTTCCATCTGTTGTTTCGCCTCAAGATGGTGACTCCAAACCCTCCTCTCTTTT
GTTGTAAGGTCTTTACTGTATACCTTTACCTTATACACACTAGTATTGGGAAAAAAGACCCCAAAAAAGT
GATTCCCAAAGACTTTATTCTCTAAATCGAAATTTCTTTTTTACCTTCCTCTATTCCCCAAAATGTCAGA
ACCTGTTCTGATTTTCTTTTAGCTGTTCCTCTTTGGACCATGATGGTCCCAGTTTCCAGGAGACGTTTCT
CAAGGCGCCTGCTGGGGTAACTAACCCTGAGGCTCACCGAGCCTTCCTCCCACCCCCTTTCCTGGTCCCC
GATTTTCTAACATTCCTCTGCTCTGGCTTGCTGTGAGACTGGCTCATTGCTGCGTTGTGGAATGTGATCC
TTTCCTCAGCACCACTCCTCAGTGTCCATTTAACCTGGTCCATGTTTGCCCACCATTCCCCATCATCATA
ACCTCTGCCAGCCCCTGTTTCTGAGCTCAGTGTGCATGTGATCACAGTGGTTACAGATGATGCATGGATA
ACTGGTGATAGTTGCAAGGGGCAGCCTTTACTGTCTTCACTTGCCTGGTTTTCACTTTGAGTGAAAAAGA
AAGGAGATTAAATCCAGAAATATAAACTATGATACTCCTTGTAAATTTAGTGTTTGTCAAAAAAAAAATC
AAGATAAAATTGTTAAAAAAATTTTTATTTCCTTTATTTTATTTCATGGATGCCAATGCAAAACATAGAC
TTTATTGATCATTTAGAAAATATATGTTCATATCATAAAGAAGAAGAAAAACATTAAACACTCATTATTA
ACTTTTGGTAAACAATCCTCCTAGCCTATTTTTGCATTCTTGTTTTTCTTATAAAAGGTGAAGAAAGCAG
GCAGATTGTACTTCGTAACCTACATGTGTTTCCTGTCTCTGTGCCTTCTTCTCACCTACTGCTGACCAAA
TTTTGGCCTGGACATTTTCTGCCTTAAAAAGTTGGTTTCACATTTCCTTACAACCGATAACTTTTAAAAT
AAATAATAATATTATTTATGTGTATGGGTAGGAATGTGGTTGGTATATATGTGGCCGTCAGAGGACAGCC
TTGAGGGGCTTCTTCCACTCTGGGATCTGAGCATTAGATTTGGCTGACCAATGCTTGTCTAGCAAGTCCC
TTTACCTACAAGGCTAGCTTATTAGCCCAGTGTTTCGTTTTTGTTTGAGAACACTATTTTAACATAGTCC
AGGCTAGCTGAAAATTCAGTAAGTAACTGAGATGCCTTGAACTCAGCAGCCTGTTACTAGGGTGTGACCA
ACCTGGTTGCTGATAGCTTTATATAACTGTCGGGAGGGTGGTTTGAGAGGAGACAGTTTCTATAGTGTAT
CTGTCATCTTGATGCGTGTCTTACTTATGATTATACTAATTATGGATATTCATGTCTTCTATTCTCTGAA
TCCTAATTTCACACCCTAATTGACATTCTTTATGTCTCTATGTTTAGAAATGCTTTACATTAAAAATGAT
TTATTTATTTTGATTGATGTGTATGATTGTCTGCATGAATATGTGCTTCATGTGAGTGTGGTGCCTATAA
AGACCAGAAGAGGGCATCAGATCGCCTTGAACAGTAATTGCAGACAGTTGTGGATGCTGGGAATCAAACC
TAGGTCCTCTGCAAGAGCAGCCAGTGGACTTAACCACTGAGCCATCTCTCGAGCCTCTGAAACACTTTAT
GCTTTGATTTAATCAGCAAAGATTGACAGTTATTTTATCATCGTAAGTTTCTCCTTGGTATAAAGGATTC
TTTGTTCCCTGTTAAAAACAAAAAAGTTATTCTTTCTTGGTATGATTTAGAGTTACTTACTGGGGTATAA
ATATGTAATTGGCTGTTTTCTGAGGCCTCTGAGAATATGCTTTCTTTGCATAGGATCAGAATTTGTCTGG
AAATGGACTTTCTTGCTGGCCTTTTCTTCCCTGGATGCTTTGTTCCAGCATCCTAGAGTTTTCCATGCCA
GTCTCCACAGCTAGTGAACACGGCCAGTCTCCACAGCTAGTGAACACGGCCAGTCTCCACAGCTAGTGAA
CAAGGCCAGTCTCCACAGCTAGTGAACAAGGCCAGTCTCCACAGCTAGTGAACACTGCCAGTCTCCACAG
CTAGTGAACAAGGCCAGTCTCCACAGCTAGTGAACACTGCCAGTCTCCACAGCTAGTGAACAAGGCCAGT
CTCCACAGCTAGTGAACAAGGCCAGTCTCCACAGCTAGTGAACACGGCCAGTCTCCACAGCTAGTGAACA
CTGCCAGTCTCCACAGCTAGTGAACAAGGCCAGTCTCCACAGCTAGTGAACACTGCCAGTCTCCACAGCT
AGTGAACAAGGCCAGTCTCCACAGCTAGTGAACAAGGCCAGTCTCCACAGCTAGTGAACACGGCCAGTCT
CCACAGCTAGTGAACACGGCCAGTCTCCACAGCTAGTGAACACGGCCAGTCTCCACAGCTAGTGAACAAG
GCCAGTCTCCACAGCTAGTGAACAAGGCCAGTCTCCACAGCTAGTGAACACGGCCAGTCTCCACAGCTAG
TGAACACGGCCAGTCTCCACAGCTAGTGAACAAGGCCAGTCTCCACAGCTAGTGAACAAGGCCAGTCTCC
ACAGCTAGTGAACAAGGCCAGTCTCCACAGCTAGTGAACACGGCCAGTCTCCACAGCTAGTGAACACGGC
CAGTCTGACATGTGTCTTCCCATCAGTCCTCTGTCAATGCTGTAGGTGGAGATTCTTCCTAAACTTTGGT
CTTTCTTTTTAGTACTTTAACAGATTTTGCCTTTTGCTATATCTTGGTATTTCAAGGGATGGTGGAGGAA
GGATTTTTAGTTATTAATGATTTGTCATTACTAGTGTCAACTTTAATATACCAAAGATGACATCTTCTCA
GTAATTTTTTAATTGAGAAATAATTGGGATGGGTGAGCATGTACAGCCTTTTTTCCTAGCTCTTACTAAC
TTTAATGGGCTTTTAATAGAAAGTATCTTAGTTGCTTTGTTGTTGTTGTGTGGAGATACTTGACCAGGGC
AACTTAGAAAAGAAAGCGTTTAATTGAGGACTTGCTTCAGTTTCAGAGGGTGATCCATGGTCATCACGGC
AGGGAGCAAGGCAGCTGGCAAGTGGGCACGGCACTGGCGCAGTAGCTGAGAGCTTACATCTCCCACACCC
ACAAGGCCCATCGAGAACGAATTACCTCAAAGCCAGTGACACACCTCCTCTAACAAAGCCATACCTCCCG
ACCCTTACTAAACAGCTCCATCAACTGTGGACCAAACATTCAAATGAGCCAGTAAGGGCCATTCTCATTT
AAACTGCCACAGAAAGTAATTGAATTTGGTTTTAGTGACTTTCAAAATGCTGTATGCATATTTAAATGCT
TTTTTAAAACCTTTAATTTTTTTTCACTTACTCTTCATAGAATATTTAAACATTGTTGTTGTTGTTGTTG
TTGTTGTTGTTGTTGTTGTTGTTGTTAGATGAAGTCTTAGTGTATTGTCTTGGATGGCCTGGCCTGGAAC
TCACTGTGTAGTATCTACCTGCCTCTGCCTTTGCCTCTGCCTTCTAAATGCTGGGGTTCAAGGCATGTAC
CACCTTGCCCACCCTTCATAGAAAACTTAAGGAGAAAGAAAAGAATACAGGGTACATAAAAATCAAGTAT
AACCAATCTATTATTCACTTCTGTCTTAGGGTTTTATTGCTGAGAAGAGACACCGTGACCAAGGCAACTC
ATAAAGAACAACAGCTCACTGGGGCTGGGGCTGGCTGACCGTTTCAGAGGTTCAGTCCATTATCACCACG
GTGGGAAGCACGCAGTGTCAGGCACACATGGGGCTGGAGAAGGAGCTGAGAGTTTGACATCTTGATCTGC
AGGCAGCAGAAGGAGACTGGGCAGAGCTTGCACAGATATATGACCTCAAAGCCCACCCCCACAGTGACAC
ACTTCCTCTATCAAGGCCACACCTCCTAATTATGCCATTCCTCATGGGCCAGGCATTCAAACATGTGAGT
CTATGGGGGCCAAGCCTATTCAAACCACCACACTACTTAGTATATTTTGGTATTCACTTTCTTTTTTAAA
AATGGAACATATATAATGTGCACATTCATATATGCATATCCATGTCTGACTTTAATTTCTTAGATATGTA
TTGAACACTTTGAGGTGCCAGGCAGTTTTAAAATTTTGCTGTATATAGTTTACATCTTGCCTACTTTCAC
TTAGTGTTAAGGACTGGACAAAACTGGTTCACCAAACTGTGTTTGTAGAAGCCCCGCTCACAGTGTCATA
ACTCTTTTCAGGGTTTTTTAGTTCAATCCCTCCTTCCCCTCCCTTTCCCTCCCTCCCTCCCTCCCTCCCT
CCCTCCCTCCCTCCCTCCTTTTCCCTCCCTCCCTCCCTTCCACCCTTCCACCCTTCCTCCCTTCCTCCCT
TCCTTTCTCTTTCCTTCTTTTTGTCATCAGAGTTATTTTTGTGTTGCTGGGACCAAAAAACCTGATGGAA
GCAAGCCGAGGAAGGATAGACTTGTTTTGTTCCATTGTAGAGGGTCCAGTCTGTCAGGACAGCAGGAGTT
CAGGCAGTGGCCTCTTCACATGGCAACAGACAAGAAGCCGAAAAGGCAGGCTGCAGCCAGTGCAGACACA
ATCTTCAGAGGCCTGCTCTTAGTGACTTACTTCTGGCAGGCAGGGTCTGCTCCCTGAAAGCTTCTTCCAC
ATCTTTTGAATCAAGACACAAGCTGGGACTGGGCACGAACTCACAGAGACCCACCTGCCTCTCTGCCTCC
TGAGTGTTGGGATTAAAGGCATACACCATCACTGCCTGGCATGACTGTTGATTATTAACCACTGCTGTCT
TGCTAGGAATTTATATTACTCACAAGTTTAGAGTCACAAATTGCTGCCGCTCCCTGCTCAGCATTCAGAG
TTATATTTAAAGAACATAAGCATTTCTGTTATAATATCAAATCCACTCCTAAGAAAACTTTCAAGAGTGC
TTATTCACTAAAAATAACAGGATTATGGTGAAAAGAAGGTTCAAGGCAGACCATTCAAGACTTCTACTAT
TTCAGACCAGAGAGAGCTAACAATAGCATAATTGGTACCTTATGAGGTAAGGTGGGTCGCCTAGTCAAAC
CACTTATTGTGGCTGAGGCTCCCTGAGAGCAGAGTAAAGCTATTAAAGTGTAAGAGGATGGAAATTGCAG
GCCTCGTGGTTGTGAGCTGTGGGGTTGGCCTGAGAGTGGTACAGGGGGAGTGCAGCTTGCTCTGCCAGGA
CACATGCACAGGCCAGACAGTGCTCCTCTGGGGATAGAGCCCTTGGGCAGTGCTCACCATTAATATTCTT
GAAATGGAGCTGAAAGGGCTTGCGTACACATGCCTGCCTCGAGGCTGGGGAGGTGGCTCAGTTGTTAAGA
ACACATAAAACCCTTGGAGAGGACCCGAGTTCAGTTCTCAGCACCCATATCAGGAGGCTAACAACTGCCT
GTAACTCTAGCTCCAGGGGATCCAATACCCTCTCCTGGATTCTGCAAGCACTTGCACTCACATGTGTACA
CACCCTTCTGCCTCCAGTTAAAAAGAAAACAAATTCTTTATATACGGCTTTATGACCTTGACTCAAATCA
AGATATTGAACATTGCCATCTTTGCAGGAAGTCCCCTGTACTCCCATCTCCCATCCTTCTTTCTAGATGA
GCACAGTTCAGAGTTTTATCCCAATTAGTGCTATATCTTCTAGAAGTTTTATCTAAATAAAGTCTAATAA
TAGTTTGCTATTCTCATGTGTCACGTTTCTTTTATTCTGAAAAGTCCATCCTTGTTCCAGAGTCTGGTTT
TGATTGTAGAGTAGTAGTCCATTGTTGAATGTAAACAATTGGTATCAAGTTCAGTGTTTGCTGAGGAACA
TTTGTGTTGCTTCCACTTCTGCTTATTAGTAGTAAAAGGCCTGTGAACATCTGCACAAGTGTTTTGTGGA
TACAGTGTTTTCATTTCCTTTGCAGAATTACCTCAAGAGGAATTGCTGGCCATAGAGTAGGTGTGTGTTT
AATTTATAAGCAACTGCTAGCTTTTCAAAGCAGTTGAGCTGTTTTTCACCCTGGCAGGGATGTTCTGAGC
TGACTTGCCAGCATCAATTGTAGTTATTCTCTTGGGTGCTAGTGGTTTTCATCGTACTTCTAACCTTTGT
CTGTCTGAAGATTAATGGTGTGTTTTAGCGCCTCTTCACATTTTCATGGCCCCGTGTGTGTCTTACTACG
AAGCGCACCAATCTCCCTCCCTTCTACTCATAGAATGTGGTTGAGAGCAAAGTTTACTTAGGTCTTGAGA
AGAGGCCTGGGCCTCTTCAGGCCCATTCCTTGTGGTTTCTCTGCACAGCCCTATCTAAAACATACAGTTT
CTTGGAAGAAGGCTAACCAGCATTTAAAGGGGCAACTGGCTTTTCCCCATAGGTTTTCTCCTAAAAGCAC
TTGTCTAGCACAGCCTTTCCACTACTTGATTTCCAGTGTGTTTAACAGTTCCACAGCTATAATGTATTAC
TATTATAAGTGTGAAATTGAGTGGGAGTAGGGACATCCTTACCTTGTTAATAATTTTAGAAATGAGAATG
TGAAAATTCACCCTTGCGCATGCGAGCTGTATGGCTTTGCAAGTGCCCTTTGGTAAAGACAGAGAAAGTG
TCTGTCCGTTTACTATTTGTTGTCTGTTTTTATCCGTATAGATTTTCAATCTGATATGCCATCACTGTTT
CGTTTCAAAACAGGGTCTCGTGTTGCCCTGGTTGGCCTCAACCTTGTTAAATAGCTGAGGATGACTTTGA
ACCCCTGAACTTGCAGCACTTTGCCTGTGCTGGAGTCACTGGTGTGAGTCACCACACCCGCCTTCGTTGT
TTTCCCTTTTCTACTAATGTGGTGACTCACTGACTTCTTAGCGGTCACTTGGTTATGCTGCGCTTCCCCT
TTTTCATGCTTTTCGTGGCTTATGATTGTCCGTACGTCTGCCCGTTGGGTATGGGGCTGTTAGTCTGTGG
CTGGGGCTCTTGGCTTTTGCTTTCTGTGTAGGAAGCTTTGCTTGGTACAGTGCTGCATCTTTTGTGTGTA
GAGGATCTCAGTTGTAAGTTTTGTTACCTGAGTCGTTTCACCTTGAGCTATAGTTGTGTGTTGTCATTCT
GCCTCATGAGTCTTTTTCTTTTTGTTACTTGACATATTTCTGAACTTTGTTTTAATCAATCTCTCTAGAC
TAACTATCTCAGGCTTGAGTGAATTTTTGTAAAAGGTTTATTATTTTTATGTGTATAAGTGTTTTGCCTG
CGTGTGTGTGTGTGTGTGTGTGTGTGTGTGTGTGTGTGCGCGCACCTCATGTGTGCAGTGCCCACAGAAG
TCAGGGAGGGTTTAATCTCTGGGACTGTAATGAATTGTTGGGAGGTGCCATGTGGGTGCTGGGAATCAAA
CTCAGGCCCTTGGCAGAGAAGTCAGTACTCTTAGTTGCCACGCCATCTCTCCAGCCCCTTAAATGAATTT
AAACTGTATTATTGTGATTACTGATGTTTTCTTTGAATTCATTTTCCTTTTCCATTCAATCTTTCCATCT
TCCTGCCTCCTCTACATCTTTCTCTAACAACACCAGCCCCCCCCCCCCCCATGTTCCTTAGCATTTTCTG
CCTTCAGCAGGCAGAGCCTTCTGCACAGGCTTCCAGCTCGCCCATGTTGTATCTGGCACCCTGCGCATGA
TGCATAAAACAGTGCTACACAGATCTCGTTTTTTGACTTATCATATCTGGGTCCGAAATGAAGGAGCTTT
GACAGATAAGCTGTCTGTGGCAGAGCTGTTGGTCTCTCTCTGTGTACTGAGGGGTCTGAGGTATCCAGGT
GACCTCCTTCATGGATGTGCCATTTAAGTTTCTGTTTGTCCTGTCTGAGCACTAGGGACATCTGGACTAA
ACTTTAACAGTAACCATGTTTAAAGGGAGTTAACAGGCCCCTGTTCTCTCCGTTCTTTGGCTCTTTGCCT
GTCTCTAACACTTGCTGATAATACCTCCTTCCCCCTTTTGTCTCAGTGCCCAGGACTCAAGGTCACTGAC
TTGGTCGCATCAGTCATTAGCTTTGTCATTACTGGTGTTTTCTTTTCCTTTTTGTTTTTTGGGTTTTTAT
TATCATTATTATCTGACATCTTTTTCACAGTAGGTTGATTTTCCACCGAAGGCAGTCCTTAAAGGTTTTC
TTCAGAGCCTGGATAGATGGCTTCGCAGTTAAGAACACAGACCCACATGTAGCTCACAGCCTTCTGCAAC
TTCAGTGCAGGTAGTCTCCCTCCTTTTTGAATCGAAGTGTGGGCCAGCAGTCGAGAAGGATTTATGTTTC
TTCTTCTGTGCTCTTTGGAGAGGCACCGAATCTAGAAGGCAGAGTGGTTTCAGGTGGTCCTGGCACGGCT
GTCTCAGAGGGGGAGCTAGGTTTTGAGTGCCAGTTTCTCTGTGGGCTGTGGAAGCTGTAGCATCCTGCTT
CACAAGGCAGCTCACATGCCACACAGGCCGCAGCTTCACGGCACCGCACACTTGTGCGTCAGTGCATGCT
CTCTGCCCTCTGCAAGAATCTGTGACACGGAAGACATTTGGATGGCTAACTACGTTTCCTTCTTTCCCCC
CTAGGTCTTGATGCAGAGCTTTACTATGTGAGAAATGACCTCATCAGTCACTACGCTCTGTCCTTTAACC
TGCTAGTGCCCAGTGAGACAAACTTCCTGCACTTCACTTGGCATGCAAAGTCCAAGGTAAGCGCTGCCGG
CAGACCACGTGACAGGTCACCCAGTCTGCAGAGGCGAGGGCTGTCCCTGAGGATGGGCCTTCCTCTCCAC
AGAAGGGTCAGTGGTGGTATAGAGCACTGGGTCTTTTCAGGTGTATAGCTGACAGATACAGTCTTGAGCC
TGCTCCTCTGAAAGTTTAGATTTAGGTGGCTGCCAGGTGGGTGACCAGCGCATCACAGGGCTGGGTCCTT
TGAGGCTTTTGCAGGGAGATCTGTACCCAGGAAAAAAGAGAGTTGGATCTGATGGTGACCCTGCTGTTAC
AGAGAAGCCATATGTGGCTGGAGGATGGGTGAGTGGCTGGGGAGGAGCATGGCTGAGCTGGCTGGCTCTT
CCACACACACTTGGTGACTTGAACGTAAGTTTCCCATTTACTGTTCTGAGTCCCCCTGGTAACCATAGAT
ACTGAAGCGGGGAGGGGGGTGCTTATCAACTTCTGCCATTTGTGTATTGGTGAATTCTTCACCTCTGACT
GCCAAGGAACCTTACATGCTTGTCTCCCAGATTTGCCAGATGATACTGTTTGTACCCTTTACTCCCCTCC
CTGCCTGCCTATGAATCCTTGAGCTGTTTCAAAGTGCATTGAACGCATCATGTCCTCTCTCTCCTAAATC
AAAACCAGGAAGTTTGGTGTTCTAACTCACAGCACTTGGGTAACTGAGTTCTAACTCACTCATCAGGGAC
CCTCAGCATCTTTATCACTAAGGTTTCACTAGAGACTGATGTTGTTTCAGTTTGTGTCAATCTGAAGTTT
TGATGCCCTGCCCATGTGCTTCAGCCTCAAGTTGTTGGGAGTGGAGAACCTGTCATGGTGGAAGCTAGCT
TGGAAGGGTCGTCTATGAATCTATATGCTTGCAGGGGGAACACTGAAGGGGACTGGTATAACTCTAATTA
TCTGGTAGATTCCTCTCCTCTCCTCCCCCCTCTCCCCTCCTCCCCCTCCCCTTCTTCCCTTCCTCTTCCC
CCCTCACCTCCCCACTTCCCCCCTCTCCTCCCTCCCCTCCTCCTCCTCTCCTCCCTTTCCTCCCCCCCCC
CCCCAATCCTTCTCTCTCTTTCTCTTCTTGAGGCAGGGTTTTACTCTATAGCTCTGGCTACATGTAGATT
CAGCTGGCCTTGGACTCAGATATCTGCCTCCCAAGTTCTGGGATTAAAGGTGTGCACCACTACACCTGGC
TAGCAGCATGTATTTAACTATACATTTGGGCTTCTTCTTGATGATATTACAGTCCCCAACTCTAGTTCTT
AAGTCCTAGTATCTGGTGATTTTTGGCACAGCTGATTAATTGCAGTTTGTTTATTTAAAAAGTTAATCGT
ACGAGTAGGTAGTACATGGATATGGCCTGTAAGGGTATACAGTGAAAAGGAAGGTTTTCCGTTGTCATGG
TAGCATGCTGAGCAAGCCCATCTAGATCACTGTCTTCATCAGGCATTGTTGCCTGAGTCTTGTCTCCTAA
AGAAAGGTAGTGTATATGTGTGCAGTGTGCACACGTACACCTAAGTGCTTCCTGCTTTCGTGTAGTGTGG
AGTTGTGAGCAATGTGTCCTGGTAAAGGGGCACTGTTCCTTGTTCCTTGGTATTGCTTTGTGTGTTCCCC
CCTTAAATAACTGTATCATTTTCTCACATAGTTGTGCTCTGGTCATTTCCTACAAGAGGAAACCGAGACC
CAGGTTTGGGAAGGACTTGCTTAAATGAGTCTGGAGGCTGAAGAAACAGGTGCTAGGGGTGAGCTCACAT
GGCAGTCACCCGAGAAGGCAGGAGTAGTAAGGTGCGGTCTCTTATCACTTGATTCTGAGATACAGTCAGA
CTTCAGAAGGAAGGCTGTCTCTCTCAGTTTCTAACACTCTGTCAGAATCTCTGTTTGGGCTGTTTGAGCT
CTTGCCAGAAGATGAACTGTTGGCTTCCGAGATCTCCAGACTCGCCAGTGTATCACTTGAACACTGCAGG
CATTTTAACATCAGTGTTCCCACATCAGAGCTTTTGCTGGTAGAGTTCTCCAGTGTTTACGTGACTGGTC
TTGCCAAAGATTTCAGTTCCCTCAGCGGCTGGAGCTGTGTGTGTCTTTTCTTAACATCTATGTCACGCGC
GCCTCGCTGAGTGGTTGAGAATGTGTTAATTGTATTTTAAGATGGGAGGAGAACTCTGGTATTGATTCTC
TCACAAGAGTAGACGACTAGGCGGTCTTAACAGATAATTAGTTTCTTGTATGTTCCAGAACATTCCCCAG
TGTTCAAGAAGAAACTTGCAACTGTGTCTGCTAAGTGTCAGTAGATATGCTAGACAGCCAAAAGGCAGAG
GCTCAGTAGGATGTGGCAGAGGACTCCACCACCCACTCCCTTGGTCTCCGTCCAGGAGATTCTGCGGGGA
GCTTGTCAGAGCTCCAGCAGCTGCCTCTGGGGGCCAGCCTGCTGTGTCCCAGTGGAGTGAAGTAGCTGTG
AGAAAGGCAGTTCTGGCAATTTCAGAGAAATTAAAAGGCCGTTGACTGTAGGCGAGCACATTCACTTTAG
ATGTGTTCCAGAAGGGGACTGCTTTATTCGTCGTCTGCCTATTCACACAGCCATGCGTTCCAAGTAAGGA
AAAATACCAAACATGTTGATTTTTTAGCTTATTCAAATATTTATATTTTCTCTAGTTACTTTTCTGAAGT
ATTTTGGCTCATTTATTTTCTGATAAACAGCTGCTGCACAGATTTGCTTTGGGGTGGGGGGCAGGGTAAG
GCTGGGGTGCACTTTCTAAATAGATCTCTTTCTTCCAAGGTGATTGACAAACGGCATTCCTTACAGATCA
GTACACACCAAAGTAGAGACAATTGTATTCCAGTGATTGGCACTTTCTTCTCTGACTCCAAAAACTACAT
TAAAAAAAAAAACCAAAAAACAAAAAAACCCAGCTGTCCTCAGTACACCAGTGTGAGGGGGGAACTGCCT
TCTGATGCTTCCGTGTTCATTCATCCGTCCAAAGGAAGTCTTAGGCAAAGGGCAGGCTTCCATGCTTCTT
GTTGTGGGTTCCTCTGGGCCACTGCTGATTCTCTCAGTCCTTCCCTCAGCCACCTCTGCCTGTGCTCTGC
TCCTTGTTTTGCCGCCACTTGCAACATCATTTCCTCCCTTTCTCCCTCTCCCGTGCTTAGACTGAGATGG
CCCTGCCACTCCTCTTGCCGCTCTCCCTCAGCTCTGCCGTCAGAGCCCTCTCTTTGCTTTCCTGGCTTTG
GTCCCCGGACAGCTATTCCTCTGCTTATAGCCAGAACTTTGTAAAGGAGACCGAAAGGTTAGAGCAGATG
TCCAAGAGGGGCACTGAGGGGTAAGGAGCTTCTGGAAGAGCATTGATGCCATGGACAGACAGAGTCAGCA
AGATGACGTGGGCCTCAAGCACAGCTCAGGCCCTGGCACTAGAAACGGACGGCCTGGCAGGGTGTCTGGG
GAGAGGCATCGAGCCCATGTGATAGAAGTGGGGTCTTTGATTCCCTGGCAGGAGGCAGAGACAGGAGGGG
GAGCTGGATCAAGTAACTCACTTCCCAGGTGAGAAATCCTGGCTCCAGGATGTCTGTTAGCTAGGAACAC
ACACTGTCCCAGTGGGGTGCCTGCCTACCTATGAGAGGAGGCTCCCCCATGTATGTAGGAACCCACAGAC
AGTATCCCAGTGAGGCGCCTGTGTTGATCTGTGAGAGGAGCTTGGCAATCAGGTAAGCTCAACTCTGTCT
GGTAACCCTACAGCTTCAGCCTTTGAGGTTACCAGATGGGTATGCCTAGTAGGTAGTTGGAGGAAAAGGA
GAGGGAGAGACAAGTTGAAATGTTTATCCTTGGGACTGGAGTGCAGAGGGTTAATGTTCTGATAGGTCAG
CCTGATGCAGAAAGGAATAAGAGTTCTTCCTGGAAGTGAAGAAGCCGAGCAGGTAGCGTGTGGTGCACCA
GCGAGGAGCACGGGTGAGGTTGAAGAGGAGCAGCCATTGAGCACAGGTCTGGGGGGTCATCATGTGTATC
ATCTCCTCAGCAAGCTGAGCCAAGGGCCATGGGGTTGCAGGCACTGCCATCCAAGTGTGAAGAAGAAGGA
GTGAACCAGCTGAAGGTGTCAGAATGACTGTGTCAGTGATGTCCCTGCTGGGGCACAGGCTGGGGCACAG
GCGGTGGGAACATGAAACCCGGCATCCTGGCTGTGCTGGGAAGGAAGGAAGTAGTTTAATTAACCACTGC
TTTCCCCCCTCTACTAGCGCAGCCCTCGGCTCATAAACATGCCTCATAAATGTTGTCCGACTTAGAGTAG
TGAGCACAAAGAACTGCTTTTACAGGGACCAGAGGTGGGTGGTCAGAGGCAGTGCTGGCATCTTGGATGG
AATGGACGGTATTTTTGTATCTGTTTTGGACATAGAGAAGTTGAGTGCTATGTCAGAACGAACACTGAAT
TTCAGAGTTAACAGAAAGATGGGAGGAGACGGAATGAGCCAGCCTTCGAAACCTGTGTAGAGTCTTGTGT
GTGACATGTGAGTGGCCCGAGACATGCACTGAGTAGAAGAGAGCTTCAGAGTAGAGGGGCTGATTTCACG
CAGGACGTCTAGAGAGGGCCTTAGAGCCTGACAGGGATACTGACCACATGTGCAGACCCAGTGCTCAGGT
CTAGAAAGTAGACATGAGCAAGAAGAGGAGGAGCCTGAGGGGAAAGCCACACTGAGGGAGATCATGAGGG
AGATGAGAGTATGAGAGGCTGTGCAGCCAGTGGGGTTCCTCACATCAGGAACAAAACCGATCCCACCAGT
GCTGACAGCTCAGGAGGGTTTGCTGCAAACCCCTGGGGTTGGCAGGAGGCTGATGACCACAAAGGAGTGC
AGCTTGACAGTGGGAGAGGCTGCTGAGGCCCCGGGGTCCCGAGCCAGTGTCACGGGCCTGTTGTGTCATC
AGCTTGGAAAAGTTGCTCATTTGTGCAGGTCACGTGGCCTCCTGGTTCTAGATCCCATGCTGATCTGTCT
CAGGCACCACCCCCCCCCCCCCCCCAAGCTATGGCCTCGTGCAGAGATAACAGCCATACCTTACAGTCAG
TGCATCCTCTTGAGGTTTTAGTGCTGGGAAGCCCAGGGTACCTTAGTGATGGCTCTCTAGCGCCCCTTGG
TGGCAGCCATGGCGATGTGATGAGCTGAGAGATTGAATGCTTCCTTCCAGACTGTTGATTTTAAGGGAAA
GGGTGGTTCAGTTCCAGGTACCAAATGGACAGTAGCCTTCAGGTTGAATTTCTGTCTGGCTGAAAAGTCC
TAGTATGATTTCAGTTTTATTATTTTTATTTATTTATTTTTGAGACGGGGTCTCACTTTGTAGCCCAGGT
TGATCCTGAACTCACTATATAAACCACACTGGCCTCAAACTCACAGAGATCCACCTACTGCTCACGGCTG
ACTGCTAGAATTAAAGGACTGCTCTTGGAATCTTTTTGGGAGTGGATTTTTATTCCACCCATGAATAGCT
CTCATACTGCTTAGTTAATGAACTTACTAATGAAGTCTTTTTCTGTTGTCAGGGGTATAATGTTTTATCT
CTCTTCATTTTGCAGGTTGAATATAAGCTGGGATTCCAAGTAGACAACTTTGTGGCTATGGGCATGCCCC
AGGTCAATATTTCTGCTCAAGGGGAGGTCCCACGCACTTTATCAGGTGAGCCAGGAAAGACGGACTTAGC
ATACTGCTTTCAGCTGCCTCCATCCTATCCCTACAAGGATAGTTCACTGAGTACCGGGCCCTCCCACATC
AATCATTAATCAAGAGAGTGCCCCACAGACAAACTCAAAGGTCCCAGGGGGTTGGGAGGCAGGGGTTGGG
GAAGTAGACGTGTAATTATTATTGTCATCTTTATTGTCTATTTATTTTTAAAGAATGTTGTAGGGCTTTT
CCTGGCAGTTTTATTCACAATTATTATTGGCACAAGTTCAGCTAGGCTTTAAGCAGAGCTGGGAGGCAAG
TTAATTTTTGGGGGTCAGCAAGAGTTTCAGAAATATCAAAGTCTCTGATCCACTACTCATAGATGGTGAA
ATATCTTTGAAATTTGTAAGAAAGGAATCATAAATTAAAAAGGCAAAATGTTCTAAAATCTACGACTTTG
TCATTAACATTCCTAGATATACCTTTAGAAGCTAATCCTATTTAGAAGGGCAGTGTTGTTTTTAATTCAT
AGCCAAGAACATGTTAGCTTTGTTTATAGGTTTATTATAATTTCCCCCAGTAACAATGTCTTTATTTCCT
AGAGGTACTGCTTTTGAATGGTACAATTTTTATTTTATTTTATTTTTTAAAGATTTATTTATTTACTTAT
TGATTCATTTATTTTATGTATATGAGTACACTGTTACTATCTTCAGCCACACCAGAAGAGGGCATCGGAT
CCCATTACAGATGGTTGTGAGCCACCATGTGGTTGTTGGGAATTGAACTCAGGACCTCCGGAAGGACAGT
CAGTGCTCTTAACCACTGAGCCATCTCTCCAGCCCTGATACGGTTTTTAAATGTGGTTACTAAATACACA
TATGACATAAACATTCTCCACAGGGAGTACTTCTAAACATGTCATGCACCTGTCTGCTCATACTATGGAT
ATAGTTGTTATTGGTGGTGTGCTGACGTGTATTTTTGGCCATCTGTTATGGTGTGGCTTCAAGCCCAACA
GGAACTCTCTGGCACCCAGTTCAGTGCAGCTTTGGTTTCCCTACATTTTCTTAGCCAGACAGCAGGAAAG
GCTTTCCCTCCTTGGGATTCGATGATGGCATGCTTTATTCCGGGTAGCTCATGTCTGCCCTGCAGGCCGC
AGTCCACACGGTGTGGTCAGCACTGCCTTCTTGACATTCATACCCTTCACGTTGCTAGGAGCGATACACA
GGTGCATTTGGTGCTGCAGAGTCTTTCCTTCCGTGGTGTAGTTTTGTTACCGTTGCTGGGGAGACTGGTA
AGCTCAGCAGTTTGCCTCGTGATTCCTGAGATTCAGTGCATTGGAAAGCAGAACATAGACAACAAACTGA
TATTAAAAACAAAGTCACAAGGAAGGCTCGGAAGTAACTATCCCTTCATCTGTGTCCTGTGCTCTGCTTC
AGTCCTCTGGATCTGCAGTGAAGGAAATGCAGGTGTCCAGAGCGGTGGTGACAGGGTCTCCGGAAGCTCC
TGTCTGATCCGCTTATTTAGATGCGCTTCTTTATTTTATTTGAGCAGAAATAGATGAAAATGTTGTATAG
CATTGATCCTAATCTTTAGGACACTAAAGTGTTTTCTCTTGTTTCTTTTAACAGTGTTTCGGGTCGAGCT
TTCTTGTACCGGCAAAGTCGACTCTGAAGTCATGATTCTAATGCAGCTCAATCTGACAGTGAATTCCTCA
AAAAATTTTACAGTTTTAAATTTTAAACGAAGGAAAATGTGCTACAAAAGTAAGGAATTTATTTAACAGA
ATGTGTTTCATGTAGATAAGAGGTCAATAGAAGACTGCATTTTCTGTATAGAGATTTAAGCATGACCAAG
TCTTGATTATAGTTATGAGAAAAGCGCATTAGTGGATGAAACTCTGGGTCAGCCTCTGGCTCTCATTCCA
TTGGAGTTCTGTGGGTGTTTCCAGAGCGAGCGACAGAGCTGAGCATTCGCTCGGTGGGCTTGGACTCTGC
AGGACCTTTGTGTTGGGGGTCACATGGGCCTGGGTTGAACCATATCTCTGTGTTTTCAGGACAGGGGAGA
TATTGCACCTCTGCAGGGCTGAGATTCTTGTCTGTGAGATGTGCATGTTGCTGTTTCAGAGAGGTTTTAA
AGGGTAACGAAACCTAGTCCAGAATTAGAAAGTCACATGTGGGCACTTAATAATTAGGGTTTGAATTATA
TCATCCATTACCCAGGAAATCACCATAGATAGTAGTGAGTTCTAGAACACCAAGGCTGAGTTTTGAAATG
TTAAGATTTTGTTTTAAGAGTTAAATATTTTATGTATATGAGTACACTGTAGCTGTCCTCAGACACACCA
GAAGAAGGCATCAGATCCCATTACAGATGGTTGTGAACCACCATATGCTTGCTAGGAACTGAACTCAGGA
CCTCTGGAAGAGCAGTCAGTGCTCTTAACCCCTGAGCCATTTCTCTAGCTCCAATTTTAAGATTCTTAAA
GGTTATCTAATCTCCATGTAGCCTACAGATAAAATTTCCAGAATTTACAAATAGGTAGAATTGTTTACTG
TTTAAGGTAGAAACAAAACAAACTGTAAACTCAGTGGATGTTTCCCGTGATCTGTACACTTTACACGCGC
TAAACCCAGCTTTAGGTGTTATGTTTCAAAAAGTCTGAGTGGCCTGACCAAGGTCAGACAAAGTGCTGCA
GCCTGTGCTCTCCACTGCTTTGTGAAACTATTTACAGTTTCCCAGTTGAAACTAGCAAGTGAGTTACAGT
GGCAGATGCCAAGATACAGGCCAGAGATACAGGCCAATAAAGAAGCTATATTGCATTGAAAAGAGACATT
TTTAAAATTAAAAAAAAATTAACACCCTTAGCTTTAGTACTGTGAATTATATACCTACTTCAATGTAAGT
TTAAAGCCTGTAGGGACTGTAGAGGCTTTACTCCAACCTAATCTTTTTGTGATTGGCATGAGGACACTGG
TCCTCGAGAGTGGGAGGCCTCCTAGGTTTGTATAACTACTCAGTGGGGATGGATGGAGGATGGATGGATG
GATATAGAACAGGTGGATGGATAGGTGGATGGATAGATAGATGGAAGGATAGAGAATGGATGGAGAGTGG
ATAGATGCATAGAAGGATGTATGTATAGATGGATGGATAGAGAATGGATGGATGGATGGATAGAAGATGG
ATGAATGGATAGAAGGAAGGATAGAAGATTATGTATATATGAATGGAAGGAAGAAAGGATAGAGAATGGA
TGGATGGATGGATGGATGGATGGATAGAAGATAGAAGATAGATGGATGGAGGATTAATACTAGGGTTGGA
CCTAAGGTGTGCTAGGAGAATAGAATGCTTTACCATTGTGCTATATCCAAAACTCATATTTTAGAATTTT
TATTTTGAGACAGGGTTTTGCTAACTTATCCAAGCTGGTTTTGAACTCACTATGTAACCTAGTCAAGCCT
TGAACTTGTGTTTCTCCTGCCTCAGCCTCTGGGTAGCTGAGACTAAGGTGTGCACCACAATGCCTGACTT
GTGAAAACTGTCCCATTTTAGATTTAATGCATATCACCATCTTAATTTGGTGGTCCTATTTTGTCCACTT
GGATTGTGTAGTTTTTGTTTCTGACTCTTACTTTTAACATGTGATTTTGTTGTATTTATCTGGTTTCCTA
GCCTATGGCACTTAACTGAACTAGTAAGCATCCCCGAGCTGTGTGGAATAGCTGTTACTTCTGGGAGGGG
GTTCTGTATCTTATGGGTGAGAATTACAGGTCAGGAAGTTTCCTAGAACTCCAGAGCCTTTCCCCTCAGC
TGGTGGCGCGGTTTCCATGATGACAGTATAAGTTTGTGGGTTTCCCTACACTCTTTACCACAGAAGCCCA
TCTCATTGGGAATCTGTAAGTGACTGGCAGAGACTGTCCTTTGACTAGTGATTGCACTGATGGCAGGACA
GCCGTTCTCATGATCTTACTGCAGACAGCTGTGACTTAGCTCACGCAGTGTTCAGGAGGTATGTTAGTAT
GTAAGGATTGTTACAGAAAAAAACTTCTAAAGTACTTTTTATTTCTCTTTTAATCCAGAACTTGAAGAAG
TAAAAACTTCAGCCTTGGACAAAAACACTAGCAGAACTATTTGTAAGTGTCACTGCGTTCGCGTTCTCCA
CCCTGCGAGTGTGAGCATGACTGCGTGAGGCCTGAAGCCGGGTGCCAGCACCAGTGTGCTGTCTGGTTTG
CAGAGTGATATGTAGACCAAATGAAGCTGTTTGAGAATTCAAACTTAAACATACTTTATAGCACGCTAAC
AAATACCTTTTAGAAGCAAGAATTTATATTGTGGTTTTTCTCAGGCAGGACCAATAAAGGAAAGGATATT
AAAAAGTTATGTGTGAGACCTCCATTAGCAGAGTGTTTAAGAATCTGACTTGTAGCCTGGGCCCAGCTCT
TAACTAATTTCATCTAGGTGAAAATGAAAATTTATGTCATTTATTCTATATTTTATTTTGATATAGCGAG
ATGACTTAGTTGGTAAAGTGCTTGCTATGTAAGCATGAAGACTTGTTCCGATTCCTAGCACCCGTGTAAA
AGGCAGGTGCTGCGTATACTCCTGTAATGTTGCACTGTGGGGGATGGGAAGGTGGGAGAAGGGCAGAGGC
AGGCAGATCTCCAAAGCCTCCTGACCTGTCAGTCTAGTTAGCTAATGCATTCCAGGTTCAGTGAGAGATT
CTGACTCAACAAGCAAGGTAGGAAGTGATAGAGGAAGATACAGAATGTCAGCCCCTGGTCTGTGCATACA
CATGTACACACATATGCACACACATAAACATCTGCGCACACACACACACACACACACACACACACACACA
CAGGGGGGGGGGGAAGACAGACAGACAGACAGACAGACAGATGAACACTCTGGTTCAGGCTGGTCTGGAG
ATCTGTATGTTGTTGAGGGTAACCTTGAGTTTCTGATTCCTCTTTCTCTACCTCAGGAGTGGTGGGAATT
GAACTCAGGGCCTCACACATGCTAGGCAAACACCTACTAACTGAGCCACACTCCCAGTCCAGGCATAGAT
TATTTAAATACATGGAGAAAAAAATTTTGTTAGGTCAGTTAGATTTATTTATTTGTTTACTCTTGGTATT
GATTATATTATACACCATTAACAAGCTTCTTTTTTCTCTTATGAACACATTATGGTAGCATTAGCACCAA
ATGATCTTTATAGAAATAGATAATATTTTCAGGAGAAAGTTGCAAGTATACTGAAGTTCAGAGTATGCGC
TGGTGGACATTTATGTCCTCAGTAACTATTGCATGTGTTCTTCTGTGCATTGCTAACTCCAAATTTGATG
GATAGGGGATTAGTGTCTTAAAGCACAGTCTAAACATCACATCATTTGACCCCTAAGTATGTCACTGTAG
GTCACCTAAAAAGCCCCTAGTTACCCAACACACCCACATGTTGATTTATCACATATATCATCTCTTCTAT
CTAATCTGTGTTCAAATTTACATAATTGTGCCAAACCATCTTTCAATAGTTTCCTTCTTCAAAATCAGGA
TCTAAATGGAGCCAATACATAGTGTGTTTTGTCCATGACACACACACACACCACCACCACCATCTCTTCC
TCCTCTTCTTCCTCCTCTTCCTCTTCTTAATTTTTTTTTATTTTTTATTTTATTTATTTTATTTATGTGA
GTACATTGTAGCTGTCTTCAGACACACCAGAAGAGGGCATCAGATCTTATTACAGATGGTTGTGAGCCAT
CATGTGGTTGCTGGGAATTGAACTCAGGACATCTGGAAGATCAGTCAGTGCTTTAACTGTTGAGCCATCT
CTCCAGCCCCACACCACTTCCTTCCTTCCTTCCTTCCTTCCTTCCTTCCTTCCTTCCTTCCTTCTTTCTT
TGTTTCTTTGTTTCTTTCTTTCTTTCTTGCATTTTTTTAATTTATTTTTTTTAATTAGGTATTTTCTTCA
TTTACATTTCAAATGCTATCCCAAAAGTCCCCCAGACCCCTCCCCCCATTCCCCTACCCACCCATTTCCA
CTTCTTGGCCCTGGTGTTCCCCTGTACTGAGGCATATAAAGTTTGCAAGACCAAGGGGCCTCTCTTTCCA
CTGATGGCTGATTAGGCTATACACCACTTCTTTATTAATTGATCATATTGTCTTTTTTTCCCATAGAATT
TGCCAAAGTAAACATTCTTCTTGTATTATTTAATTGCCTTTAACTCTTTCTTATAACTGGTAGTTAAATT
TTATACTTGATTAGACTTAGGACCACTTCTTTTGGTAAGAGCAATTCACGGGTGTCCTGTGTGCTTCTTA
TTTATTGTTTTAGCACCTCAGAGCACCTCAGAGCACCGAGCATCTGTCCTAATGTAGGCTTTTGTTTGTT
TGTTTGTTTGATTGTTTCTGTTTTTGTTTTTCTCTGTGTAGCCCTGGATGTCCTGGAACTCACTTTGTAG
ACCAGGCTGACCTTAAACTTTTTCTCCCCTCCCCTCCCCTCCCCTCCCCTCCCCTCCCCTCCCCTCCCCT
CCCCTCCCCTCCCCTCCCCTCCCCTGTTCTCTTCTCTTCTCTTCTCTCTCTCTCTCTCTCTCTCTCTCTC
TCTCTCTCTCTCTCTCTCTAATTTAAATGCTCACAAGGTATTTCTCCCTAAGAACAACAGTAGGATGCTC
TTGGCCTCTGTCTCTCTCTTCCTTGCTCTTTCTCTTCCAACTTTTATCACTCCTATCCAGCATTTACTGA
GTCTGCAGTATACTAGGTGGTGTGACTGAGGCAGAGGATTGAACTGAGAAACAGCCCCAGCCTTTACGGA
CTTAAGAGTGTTCCGGGGGAGATAGGACAGCATGGTAGGCACCCCGAGGAGGGACTTGTCACTGCCTCTG
AAGGAGAGCGGCAGCAGCTAAAGGAAGGGCAGCTGTGTTCTGACAGGGGAGTGTCAGAGAGGAGTAGGAG
CAGAGCTACACCCGCTGGAAGCTGAGGGGCTGGGCAGAGTGCGGGCGGACGGCCTGCCCAGCGGACCTGT
CTGCTCCGGCCACTGCTTGGGCTGCAGTGTCCATCCACGCAGGCTTGGGGCTTTTCTACCCCTCCTGAGA
CGTTTCAGCAGATACTCCTGGGAGTCTTGTTTTCACTTGACCCAAATCTAGCTGTCCCACTTGCATTAAC
CTTTTAGATTTTTATTCACTTTTTGTTCTTTTACTCCACGTTTAATTGTTTGGTTGTTGGCAGCTTAGGA
TCCGTCTGCTCAGCCTTCTTCCCAGTGTTAGGCTCCCGAGCTTGTGCCGCTGTATCCAGCCTGAGAGAAG
CCTGGGTTCTCCTTTACTGTTAAAAAACAAATCACATATGTATACAATGTATTTTCATCTTATCCACTCC
CCCTATAGTTCCTACTGGGTTTCCCATACACACACACAATCCCTTCCCGCCTCTGTCTGTGTGTGTGTGT
GTGTGTGTGTGTGTATGTGTGTGTGTGTAGTATGCTCACTTAAGCATTCAGTTAATTTTTCTATTCCTGT
GGTGTTTCTTAAAATAATGTAGCTGGGTATAATCATATTTAATCAATAGAACCCTTTTAAAAAATCAAAG
AAGCAAAATTATATTGATCAAGAGAACTGTGCAGTCTGCCCAGCAGGGTGACTATTAGCTGTGTGTAGCT
TTGAGGCTATAAATATATTAACACAATATAAAGTAGAATACAGTGAAGTTATTCTGGTTGTTTTTCATAC
CACATAGTTAAGGTAGCAAGTGGCTACTGTGCAGGGCAGTGCATTCCATCTGTTTCTGCAACTATGCTAT
AGAACATTTACTGAAGGACATGCGTGTCTTGTATACTGGGGTGTTTTGTATCTACTGTTTGTGAGGGTCT
GACTGTGGTTCATATTAACCAGAAATTTTAAAGTCTTAAAACTGTAACAAAACCACCAAAATTGGTGTGA
GTACCGCACTGCGGGTATCTGGTTTTATCAAAGGACGGGTGTGATGCAGTGCAGGCCTTCCCGTGAGGAG
CAGGGCACTCCCCGTGTTAGGCCACAGAGCACCCCATCAGCTCTGTAAAAACCTCACGTGCCTCTCATTT
CTGTTTCAGATGACCCTGTCCATGCAGCGCCAACGACTTCCACGCGTGTGTTTTACATCAGTGTAGGGGT
TTGCTGTGCAGTGATATTTCTTGTAGCAATAATATTAGCCGTTTTGCACCTTCATAGCATGAAAAGGATT
GAACTGGATGACAGGTACCGTTCATGTTTTGGGGAGCATGTTACTTGCATGTGCATGAGTGCCCCTCCCC
CAAGCCCCCCCCCCTCCATCCATGCACATTGTGCAGGGGAGGAGGGAGAGTGATGAAGCTCAAGCCAAAG
AGGAACTCAGATGTCTCGGTTATTACTGCCTTTATGTTTTTATCTTCATCTTTTAAAGTTTGTGGAGTTA
TAACCTGGGGGAGAAATAAAGGTCAGAAATGTTTTTCATGTTTGTTATCCTGATTCGCCTCCAGGTCCGA
CTGCCTTTTTTAAACTACTTCCTTCTACGTCCTCCAGCCCCTCCCCGTCCCTCCTTCCATCCCTCCCTTC
CTCCCTCCCTCCTCCCTCCCTCCTGCCTTCCCACTTGAGTATCTGCTGTACTTTAAAGTTTCTTGGATGA
TCCTAATGTGTAGCAGAGTAGAGACACCGGGTAGATAATTACACTAAGTGTTGGGACTATAAAGATTAAT
GATACTCTTCATGGTATCAAGGTTCTTTGATGTTAACCCATAATTATAGTATAGAATTGATGATTGTGGT
ATGTATAATGTAGTGTGGGAATGCAGAAGGTCCTGGCTTAACCTGGGTGAGTCACACACATTATAGCCAG
ACTATAGTGAAGGGTAAAGATGGAATGGGAGATACGTGCTCATAACCCACAATCCTTAGCGACAAAAGGC
ATTCTCAGCATTGTTTTTGACTTTGAACTTTGAACCCTTTCTACCTGTCTTGATCCCTAGTTGCAACTTG
TCAGGTGATTCCATATCCCTGATGCTGCCCTTTGAGAGCTTTTGTTCATACTGGGGCCATGGGACTTCTT
GCTAATCCAGACCCATTATTTGTACTCCAGATCCATCACCGTCTTTCCCACAACTAAAATAGAAGCTTTT
TCCAGGCAGGGAGGAGAGACCCTTCTCTCCCCAAACTCTCCTAACACTGTACAGTAGAGCCATGATATCA
GCATTCCCTTTGTAAAAGATCTGGGGGCTGGTTGGATCCAGCACTCTTTAAGTACTTTGGATCCAATACT
CATGACTTAGTCTCCACTGTGCTCCTTCCCTTCCTTGAGTCAAAAGAAGAGAGGAGTGAACATGGCGGAG
TGCATACAGGCACTCTAACCTGCTTTCTGTTGCCGTGATAAAACACTGGCCAAAACCATTGTGGAGGAGG
GATGGGTTCATCTCAGTTCACACTGTCAGGTCATAGTCTAGGGGATGTCAAGGTAGGAACTGGAAGGCTG
AACCATAGGGCATGGCTGCCACTGCCTTGCTTCCTTAGGCACTGAAGGCCCACCTGCCTAGGGGTGAAGC
TCACGGTCGGCTGGACCCTCTCACATCTGTCATTAACCAAAACATCTCGCTTACATAGTCACGGGCCCAT
CTGATCTGGGCAGTTCCTCAACTGAGACTCCTTCATGAGACTCTAGGCTGTGTCAGGTTGGCAGCCAGCT
CACTAGGGTAGTGGATGCTATGTTTCAGAGTGTTTGGTGTACACCGGGATCATGGAGTTCTTTGATTTTA
CAGAGGAAGAACTGGCATTTGGAAGCCGTTGAGGGGAGAAAAGACTAGACATCTTGTAAACAATTTCGAC
AGATGATATAAATGATGAGAGTTACTGAAGAGTAGAATAGTCAGAAGTCATACTTTGTGCTTTTTGTTTT
TGGAGGCAGGGTCTCATGTAGCCCAGGCTGGTCTTGAACTTTTGATTCTCCTGCTCCACCCCCTAACAGC
ACATACTGTTATACTCAGCTCCACAGGTCTGATACTCATCGAGTTGAGTTTTCCTTGACTGCTGGTGTGT
GTGTTCAGGCCATGGGTTCACACAGGCCGACATTGTGCTGTAACTCATAGATTCCATGAAGCATACGTGT
CTTCCTGCTAAGTATTCAGCACTCACAAGCAAACTCTCTAGGTTTCTAGATACTGTCTGAATAAAAGCAA
TTATGTGATTGCTTGTTTTCAGGGCACAGCCTAGTAGTTAGGAGCTAGCCTCTGGGGCCAGGCCATCTGT
ATCTGATATCCAGCTGTTCTATTTACCAGCCTCTCAGCCTTCTCACGTGTAAAGCAGAGATTGTGGGCTG
TGAAGAAAGGACGCTAGAGGGGTGGGTAGCAGATCTCAGCCGTAGCTACATGCAGTAAGTGCCGCCATGC
CGACGTCATCCCCAGAACGATACAGTGGATAATCTGGCGTTCCCATTATTCCTGGCTTGCTTACAACACA
CCAGGCAAGCTAATGGTGGTGGCCAGCACTGGAGACACCTGCTGCTCCTCTCTACACTACATGGTTGTGT
AATTAAGACTCAGAGACAGACGTAAAGACAGACTTGTGGGATTGTGGCAATACACCAGTACAAAGCAGGA
GGGCTCCATTCTGCCATTTCTGTATTGTAGTTACATCAGATGCTTGCTGCCTGACAAGCTCTGTTCCTCG
GGAGGCTTAGTGGTGCTGTGAACTCTTGGGAAGAGCCAGTGTTCTGTTACATATGAGGTTTCTCTGGAGG
CAGAGGGTGTAGCTGTCTGAGACGTGCTCTCAGCAGGGACACATGGAGTTCGCGCGCGAGGCTTTCCCTC
TCTCCCAATGCAGCACATTGATCTGTAGTCCATTACAGCAGAGTCTGCCCTGAAGGGGCTGCGTGGTCTC
CTGCAGAGCTCGGTCTGTAGCTGCTCTCTCTGTGGTCTCCCCCTCAGGGAATGGAAAATCCATTCCTCGA
GTATGGGGTGCTTGCATGTGTTATTTGGTAGTATTTTCTTTTTCTCTTCTACTTACTGCTTCCTAGCACC
TCACTACCCTCAGCATTAGGGTGGGAGTGGGGGCTTTTTGTTTTTTCTTTTCTATATTTGTTTTTACCCT
TGCTGTCGTAACAAACTGGCATACATAGTAATATACAGAGTCTTAAAGTTTCCTCTTCCTTCTCCTCCTC
CTTCTCCTCCTCCTCCTCCTCTTCCTTCTCCTCCTCCTCTTCCTCCTCCTCCTCATCTTCTATCTTCTTC
TTCTGACAGGGTTTCTCTGTATCTGTAATTTGCTCTGTAGACCAGGCTAGCCTTGAACACAGAGATCCTC
CTGCCTCTGGCTTCTGAGTGATGGGATTGAAGGCGTGCATGAAGAATGAAAAAGTCGGTTACTGCGCTCA
ACCGTTCCCTCCGCATCACGTATCAGCAGCTGGAGTCGGCAGCAAGCATTCTGCCTGGCCCCCGTCTTCC
CAGTCCATGGGCGTGGCTGCTTTCACTGAGTAGCTAACCCACTGTGCTTTGGGCTCTCACAGGGGTCCCA
GTTTGCTTCCTGTGCTGGGCACCGGTGTAAGTGTTGGGGAGGTGGAGTGAGCAGGCACTTAGCTGTCCCC
ATGGAGCTTGTGGTCTGCCCAGATGAGACGTTCAGCAAGAAGTCCCAACTGGAAAGAGGGTGGGAAGGTC
TGTGGTGTTCAGTGCTGCCTCCTCAGCAGCAGAGCACCTCCTGTCACACACAGACACTTGATAAATATTT
ATTGAATAAATATATAAATAGATGACTCACTGAAAATAGACAGTAAGATTTATTATTTTGAAGATCTTTG
TTTAAATTCTTAGCTTATCCTAAATCTAGGTGCTTTCTTGCTTATGAAAAATCAAATTTATTTAGACTAG
TCATATGCTACTAAATGTTAGTAAATATACATTTTAATCTGTTTCCTACCTCCTGTGATGGTGTCGGGCT
GTCCTTGCTTCTCTTATAATCGTACGTTTGGCTCCAAAGAAAGTTTCTTGCTCCACTGTTGCTTCGCTCT
TCTTTCACTTGTTCATTTCTGTTAAGGAAATGAAACTCTGACATAACTAGCCATCAAGCTTGACTGCTCT
CAGTCATTTTTAGCTGACGCAAGAGCACCACCTACTGTTCAGTTGTCAAAGTTTACTCACCCGGACACAT
TGTTGCTCGCAGGGAATATCTGAGTTTTCACAGATATCATACATTTGAGCCTCACCTCCTGTGAGGCAGG
TCTCCTCAGTCCTCTTCTGCGGAGGAGCTCTTATCTGTCCCCTTCTGTGATGCAGCCCTTCCCTCCTCTC
TGCTGAAGACTACACTTCTAACAAATAGGACTTTTAGTCACATCTTTGCCAAGTACTCTGCAAAAACATG
TACCTGTAGGTCACCGTTCTAAGGTTGGAGACATGATGCGCAGTATTCCTCATGATAAAAGAGTGGTCGT
TTATAAATGCATGCCTGAGGGCCAAGCAGTGACCTCAGTGATGTACTTAGACATCTTCCGTGTAAGACCC
TTTTAGCCGTCTTGTTGGGAGTAGCTTTATGCTTCCTTCTATCCCCACAGAGTTCGAGGCTCCAGAAGGA
TTGAATACTTTGCCTAAGCCACACAGCTGGTGATGTGGGCAATTGTGATTTACAGGTTTATGCTCAGCAT
TTGCTTATGACCAACTACATATGTAAACTCACATAAATCACCATTTAGTAACTTCCAGAACTCCAACCAC
ACAGTAGCCTGACAAATCTTTGTTACATTCTATGAATTGAAAGTCCCCTCAGCAAAGTTGACTGTAAGTT
CCCAGGAAGAAATAACAATCTGAAATGTGAGCGTCTCCAGGGAAAGAAGGCATGTTGAGGGCACATTAAA
TGAACAGGCATTTCACGTGGAGATTGTACTGTGCAGAGTGGCTCATGTGCACACAGGCTCTCTGAATGTT
TATTGGAGGACACCTCTATCTCAGTACTTCAGCCATGACAGTAGGTCATTCTAAAGTGTGTTAGCGATAA
AAGGCTACATAGCCTCCAGAACTCACTACTTAGATGCCTTATGTCTGTACGTTTTTCACATGTGTGTTTG
GGCTCTTGAAATTGGCAAAATACAAATCCATGTGTAAGTATCCAGATTATATAGCAGGTGGTCACCAGTT
CCATTGTAATCACTGGGGTTTGAAACTACGATGATAAGGTACAAATATCAGTCAGTCACAAGGTTCTTCA
TATGTAATAGTGGGCTCCACCACCTCTGTGGTGACAGGAGTTCTGTCTGGCTCTCTGGTTTTACTCTCTG
TGCTGAATCTCTGCTTTAACATCTCCTCGGCTACAAAATTAAAAGGAATAGGCCAGTGAGATTGAAATGT
TCCCCAGAGTGACCTGGGACAAGGTTTCTACCCATTGAGAAGATGGAAGAATATGCTTGGGAAGTGTTGG
TTTATAGGAATTACAACACGTGAGTGACAAGATTTGCGCCCACCACCTTCAGGGGCTCCTCTTACTTGGA
AAGTATGGGGTGATGTGATGTTTCAAGTTCTAGAGATTTCTGAGAACCATGAATTCTAAAAGTGGCATGG
GAGGGGTGAAGAGGGACAACATATATGATTAAACAGTGAGACTGTGAGGACACCCCTGCCCCCGTTGAGC
ATGGCACTCTTGATTTAATCTGTGGCTGCTTCAACTCAAGACCATTTGATTTGGGCCATTGCTGAAATGA
ACTTGTTTTTCTCCTTTTCTGAAAGCCAGTTCAACTGAACAGCGTGGGGTTTTTTCTAGAAAGCCGAGAC
ATAGTTCCTGACACACCCTGTGTGAGGTTCAGAATTGCTCTCTGAGCACGTCTACCCGCACTCCTGAATA
ATTTTGCTCTGATGGCATGCCCTCCGTCGTCACGTCCCTCAGGAATCCAGTTTGGAACGCTGATAGAGAT
GCGTGCCTTTATTGCATTGTTATCTCGACCAACTTATGTACCTTATATGTATGGGAAAGACATCTCTCAC
TGAGGCTCACTTATCAAGTATTTCTGTCTTCAAATTCTTTGCAGCATCAGCGCCAGCAGTAGTTCCCAGG
GGCTGTCTCAGCCGTCTACCCAGACGACCCAGTATCTGAGAGCTGACACACCCAACAATGCAACGCCTAT
CACCAGTAAGAGTCAATTGAATCCTAAAGCTGTATGTGCTCTGTCTGTGGTTAGAGTATTTGTGGGATTA
ATGTACCAGTGTGTAACTTGTGTAAAAGTTGGTTTGATCTTCGATTTTGTTTTTTTAGAGCACTGAGTTA
AAAAAAAGTTGGGGTATTTAAAGTAAAAATAGTTGCTAGAGCTAGTTTTGCTTTGGGGAGATTTCCATAG
TTAATTTTATTAAAAGAGCTAATAAATCAATTTTATACAAATCCAGACTGAAGGTTTTCAGGTTACTTAT
ATGGAATAAACTAATACTCTCATGGGGTACATAGAGACTTGGCATTGGTTTTACTCATCTGATGTTGGAG
ATAGAAGCCAGGGTCTCCAGCATGCCCGCTGTACTGTGTAAGGAGCTGTCCACCAGTCCATGCTGCTGTC
TTCTAATCCTAATTCAAATCTCATTACCATTGATATGGTCTGCCTGTCTGTCTATGATTTTTCAAAACAG
GGGTTCTCTGTGTAGCCCTGGCTGTCCTAGAACTTGCTTTGTAGACTAGGCTGGCCTCAAACTCAGAGAT
CCTTCTGCCTCTGCCTCCTGAGTACTAGAATTAGAAGTGTGTCACACCACAGCCCAACTTTTTTGTGGGG
GTGTTTGGAATTTCCTTTCCTTTCCTTTCCTTTCCTTTCCTTTCCTTTCCTTTCCTTTCCTTTCCTTTCC
TTTCCTTTCCTTCTCCCTCCCTCCCTCCCTCCCTCCCTCCCTCCCTTCCTTCCTTCCTTCCTTCCTTCCT
TCCTTCCTTCGTGTGCCATGCTGGAATCAAACCAAAGGGCCCTCACACATGTTACAAGTACTTTACCACT
GAGTGACATCCCTAGCTTTTAATTTCTCTCTTTAAAAAGAAAATCAACCTGTGTGTCTGTGTGTCTTTAT
ATACATGAGTTCTAGTGTCCACAGATGCCAGAGGTGTGGGGTCCCCCTTGAGCTCGAGTTCTAGTCAGTT
GGAATTGAACTTGGGTCATTTGGAGGAGCATTGAGTGCTCTTAGCCACAGAGCCATGTCTTTAGCCCTAA
TTTTCTTCTTATTAGCAGAATTTTGTTTTTTAGCTTAGGCTGGCTTGGAACTCTAGCATGTAGATTAGGT
TGGCCTTATATGGGCCACCATTACCAGCCCATTTTTATTTTTATACACTTCAGGGTTAGGAATTTTGGGG
AAAGTTTTTTTCTTTTTTCTTTTTTTTTTTTTCATTCTTTCTTTTGGTTTTCTTGAGATAAGGTTTCTCT
GTGTAGTCCTGGCTGTCCTGGAACTCGCTCTGTAGACCAGGCTAGCCTCAAACTCAGAGATCTGTCTTCC
TCTACCTTCTAAATGCCAGGATTAAAGTTGTGCATTATTACTGCCCAACTAAAATAAATCTTTTAAAAAT
ATTTAACTGACAAGTTATATACAGTGTGATATAAAATATGCATGTAATAATGTAGTGTTATTGGACTATA
CTTTAGATATAGTGGGGAATCAGTTATGAGATGATGATGAGAATAGACTAACCTTCTGAAACTGTAAGCA
AGACCCCAATTAAATGTTTTCTTTTATAACAGTTGCCTTGGTGATGGTGCCTCTTTGTAGCAATAAATGA
CCAACACGGGGAGGAAGATAGCCTTGATCTAATGTTATTACCAAGAGAATGTCAGTGTAAGGAAGACTAT
TGATGTCCTATAATTAGTAGCCTTTCCAAGGCAAGACCTGAGCCAGAGAGAGAGAGAGAGAGAGAGAGAG
GGAGAGGGAGAGGGAGAGGGAGAGGGAGAGAGAGAGAGAGAGAGAGAGAGAGAGAGAGAGAGAGAATGCC
TAGACCTGGCATGGGCTTTTGAAGCCTTAAAGCCAACCCCATACCTCCCAGTGACACACTCCTCTAACAA
GGCCATACCTCCTAATCCTTCTAATTTTTACAAACAGTGCCTGGAGACTGTTTAATTATGAGCCTATGGA
GGCCGTTCATACCCAAACCACCACACTCTCAGTGCCCTGCGGTGGTTTTTCTAAACTGTTTCCAACAAAA
AGAGTCAAACGTGTTAGGAGAAAAAGCAGATTCCAGGTCTGGGTCAGGAAATGAACAAGGCAAATCTAAG
ACATATGATTGAGCAACATATCCCACTTACCAAGGATGGAGCCACTGAGTATACAAGAGAATAATGACTG
CAGTGATTTGGAATAAAATATGTGTAATAATCCATAAATGTATAATGCTTATAATTGCTCAACATAATTA
TTAACTGGGGCTCCATCAATACCTTAGTATCTGACAGCTGGCTGAATGAATTATATTGCAGGGTAATGAG
GTAGCACCAGTTGCTTAGAACAAGCATTTCCTTACAGAAGGCTCCCAGCTAATGACTATGAAAGGAATGG
TACAATTAGACAAGGACATTTAATAAAATACTTAGTCTAGGAAAGACCATCATTGATGAAAGCCCTAAGC
ACAAAGGAAATGGAGGCTTCACCATTGGCCAGTCAAATCCTTCCCTGATCCACTGATAGATAAACTTAGC
ACTACAAAAAGTGCAACACCCAGGTATTTTATACCTGAGGATATGAAGTTCCAACCCCAGATAATTAGCC
TGGATTTAATCAAGGCTAGAGCTTGGAGCTGTCCTGTAACACAGAGACAGAGGAAGAAGGTGAAGGACAT
GCCAGGAGCAAGGAGGGCAGTTCTGCATCATTGATTGTGGCAGGAAACAAGGGCCAACAAAAAACCTCAG
AGAAATCAGAGCAGCACCTTTGGTCACTCTCAAGAAAGAAAGATGACAAACCCTTCGTGAAAATGCCGTG
GTAGGTAGGAAGGTCATTTCTGCTCAGAAAATGCAAACCTTTAATGTCGCTTTTAATGCTGACATTAGGG
CTGTCCTTACTGATGGATATGACCTTATGAAAGAGACTATATACCAATGAAAATAATTTACATATTAAAA
ATTTGTTCTTAAGGTGAAATTTACCTATTTTTTGTTTTTCACTGTGGACATTATCACAGAGAAACACGTA
GTTCATATATCAATAGACGCTAAAAACACTGGAAACCTTTAACTTTTACATGTATGAGTGCGTTGCCTGC
GTGTGCATGTGACCATGTGCATGAAGTCTCTGCAGAGGTCAGAAGAGGGCGTTGGGTCCCCTGGGACCAG
CCTTTACAGCAGTTGTAAGCTGTCGTGGGTACGGGGAATCCCACCTGGGTCTCTCTAAGAATATCAAGTG
CTCTTAAATACTGAGCTTTCTCTCCGCCCCACCCCCAACAAATTGTCCTAAAAACTCAATATAAATACAG
TTGTTTTATGACTAAAAAAGGAAGGCCAGGCATGGTGGTACACACTTTTAATCCTAGCGCTCAGGAAACA
GAGGCAAATAGCTCTCTGTGAATCTGAGGCCAGCCAGCTCTACATAGTGAGTTCCAGGCCAACCAGGGAT
ACATAGTGAGACCCTACCTCAAACAAAACAAAATAGGAATAGCAACAAAAGGGATGGGGTGTAGTGGATC
ATACCTATAACCCCAGTATTTGGGAAACTGAGGTAGATCATGGGTTCTGAGCCAGCATGGGCTGCCTAGT
GAGACTTGTCTCAAAAAGAAAAAAAGGGGTGGGGGCAGAAAACAAACTTAAATACTGGGCTTCGGGAGTG
CTAACGAGAGTGGTCAAAGTGAAGATGTAGTTTATCAGAAGTGATGCTTTTCAAACTTCTCCGTATTTGA
AAAGATCGTTTGTCACGTGATTTATTGAAAAATCATTTGGGAGGGAAAACTAGAGAGGGATTAGAGTCTG
TAATTCAAACCTTGTGTCATTTGTCCCAAAGGCATTCCAAATGACAATAATATTTTTAGTGCTTCTTGTT
TTAACATTTGGAAGTAGTGGTGTCATAGATGGGACTGTTTTTCCTTAATTTTTTAAAAAAAATTATTTTT
ATTTTATTCATACTGATGTTTTGCCTGACTATATGTCTGTTGTGAGGGTGTCAGGTCCCTGGATCTGGAG
TTAGAGACAGCTGTAAGCTGCCATGTGGGATTTGAAGAGTAGACAGTGCTCTTAACTGCCGAGCCACATC
TCCAACCCCTTCCCTTAACATCTTATGTGCCCTTCAGCAGCAGCTTCTCTGATCTGTGTGTATAGTTGCT
TCATTATTCATTTGCTTCATTATTCTTTGCTATGACTTAAGTCCACAGGCTGGAGTGCCTAGGTGAGCCC
AGTGCTGCAGTAGGGTATTTATATTGCTTGGTCTGGCTTTGTTGGCTCCTCAGGTTATCCTACCTTGCGG
ATAGAGAAGAACGACTTGCGAAGTGTCACTCTTCTGGAAGCCAAAGCCAAGGTGAAGGATATCGCAATAT
CCAGAGAAAGGATCACACTGAAAGATGTCCTCCAAGAAGGTACTCGTGCTCGCTACAGTTTCAGTTCTGT
TAAAAAATTGTCTTCATTTTCTATTTCTTTAGACTTTATACAGTGTCGTAGCAGCTTTGATGAACATCTT
GATTTTGAAGTGTGATAAAACAGACAAGTATTTGTGACCTATAAATGTATTGTATCCTTATTTCAAGTGA
TCAGTACCCAGCACCTGTATTTACAATACTTAAATTAGTTTATTCTTTCATGGCTTGTGGATGATCAGGT
GTGTGGATGGGCATTGGGGTTAGGCTCAGCGTTTGTAGCCTGAGTGACCTTAGAGAAACCACACAGTATC
TCAGAATTTCTGTAGAGTTCACACCATGCTAGTCTTCATCACTCTGAGACTAGCTGAGAGGCGACATGAA
ATGGTGCATTCGGAAGACTTCTCAGAGTTTGTATTTGACATGTCCAGGGCTCTTTTCACCCCGTCTTGCA
GGCCCTTGATCTCGGCTTTGAGTTTTAGGTCTCCATTAAGGAGATGGTGAGATCGGAGGACAGAAATTAA
CAGTGAAATACTGCATCACACGCGGTTAGATGGCAGATGCTTGTTTCCTCGCTGCAATTTGTTCTGGAAT
GTGTTTGGTCCGCACAACTGTTCTGTTTGCTGGGCCTCCTGGAAGCTAGTCTCAGCTAAGTGAGGACCTA
AGATGCCTTCTGAGAGAGTGGCGCTCAGTTGACGCAGACTCTTATTTCTGTCTGTTCTTCAGAGTAGGAA
CACTGCTCCTACGGTTGTCTGCTTCCTAACTCAAATAACTAGATAGATCATCTGCAGTCTGAGGAGAGAA
ACCAGGCCAGACAGCATTTCCTACAGAGTTAGTGGTTATGCTTCCAATTTCCTGTGTTTCCAAACAGGAT
GCTCATACTTTGAGGAAATTTGGAGCCATGTTTTCCTTGATACTTTTTGGAGCCATAATGGTTTTTTGTT
TGTTTGCTTGTTTTTTGTTGTTATTGTTTTGTTTTTTAAGAACTTTAGCTTTCAGAAAAGAAAACATGAC
TGGCTAATATTTTGAAAAGTGGTGAGGTTCACTGGAGATCTGACTAAGTACAAATCAAACAGTGGTTACA
CGCCACTTTTCATCTGTTAGATTGGATGAAATTAAAAAGAGATAGCTAGAGATCCAGATGGGGTTACAAG
GTGATAAACTGTTATGCACCCTGACCGGTGGCAAGGTAATCTGTGTACACATGTGCTGTAGTGTGGGGTT
TGCCCAGTTACTTTTGCATTTAAAGTTTGTGTTTGGAATATCTAAAGGGGACTAGGTTTAGGGAAAAGGA
GACACAGCAGGGTGGACGGAAGGACCAGGTCTGTCCTGCTGGGCAGGGATAGAAGGAAGGACCAGGTCTG
TCCTGCTGGGCAGGGGTGGAAGGAAGGACCAGGTCTGTCCTGCTGGGCAGGGGTGGAAAGAAGGACCAGG
TCTGTCCTGCTGGGCAGGGGTGGAAGGGAGGCACTCCTTTGACAGTACAGAGACTCGGAGGTGTTTGGAC
AGCTCCAGGCAAAGATTCTCCAAACCCTTCTGCCTGCAGCACCGATGTCACTCAGACTCTGTGACTCAGT
GGGACCTTCCACATGAGCACTGGTACCTTTAGCACACACAGTGAGATATTAAAGCCAAGGTGTGTGTAGT
GTCCACAGTAAAGCTAAACTCTTTCAGGCATTTCTTTTAAATTATGAAAAAAAAAATGGTCTGTGCGAAA
GAATAGCATGTCAGCAATCTTTTTTTTTGAGGATCATCTCATTTAAAAAGTATTTTTAGCAATCAGGAAA
TTAATAATAAATCTGTAAGGATTTTTGCATTTCTGTTTTACACTGCTGTGCCCCTCGGGAATGTCATAAT
TTCTTTGCTGTGAGAGCTCCTTAGGAGCACACTTGGCCCTCAGTGACCTGAGAGTTGGGTCACCTCACCT
GAGACTGAGGGGTAGAGGCGCCGAGGCCTCTCTGAGCCAGGGTTAGCGTTCCTCACTCTTTCATCATTGA
AGTCTGCATCGCAGAAAGAGCGGCCGTTCTCTCCCTCCGTCGTTGCTTTGAGGCAACTACCAGAGACTCA
GATGCTCTGGCTGGCACCTGTAACTGCGACAGCACGCCTGACCGACTGTGTTTATTTAGAGAAATTAAGG
AAAATAGTCTCAGATATTTAGTCTAGCTATAGGATTTCTCAAGCAACCTGCTTTGTGGGCTTGAATTGCT
CTTAACAGCTTTAGTCCGGTTTATTCTGGAGGGAGAATGAACAGCAGCCACACCTGTCAGCTTAGTCGCG
GTTACTAGAAAGCTTTGTGATACTGTGAAGATTATTGTATGGCCTCATTAACTGTTATTTTTCCTTTTAG
GTACTTTTGGGCGTATTTTCCATGGGATTTTAGTAGATGAAAAAGATCCAAATAAAGAGAAGCAAACATT
TGTAAAAACAGTTAAAGGTAGGGATTTTTACATGCTGTTTTCATAAGAGTTTTTTGTTTGTGTTTTTTTG
TCAATCACTCAAACTAGCAAAACTCTGTCCCCTGTCTCAGTGTGTGCCAGACCTTTGGAATGACTCTGTA
CTAGTGTGACTCACAGGCTAATTTTTGCAAATTAGGTTTTTCTCACCAGGTATAGAAATTAGTTTTCTAA
CTTACTAAACATTTATTAGAAATGAATCCAACAGTAGTTAAATTGCATAATTGCATGCGCTTAACCTATC
TGTGTCTAACTTTGAGGTTTTTTTTCCTGTTGAAACTCCCTTATACCATCCTTAATCATATGTAAGAAAA
CATTGTCATTAGTTTTCAACATATTTATAAAGAAGATAATGAGCTGTGCTCAGATCCACAGTGCCTCATA
TAGATATTTTTTTACATTGTTTACCTTTTGTTCTCTCCCCCACTTGATCTCTTACACGCTGTGTAGTGCC
CTAAGACATGCCTGTTTCTTCAACCCTTACCCTGACTGCCACCCTTTACCCTTACCCGTGTGTTAGGTAA
GCAGGTGTTCATGTACATGTGTGCATACATGCAGAGATCCCAGGAGTCAGCCTTAGGATTCTCTAATCAA
GTGTTATCCGCCTTTTTGCTTTCTTTTTAAATTATCTTTATTTTATTTTAGTTTAGTTTATGTATATGAA
TGTTTTGCCTGCATGTGTGTATTTGTACCACCTGGGTGCTCTGGGCCCCTGGGAGGGTGTTAGATCCCCT
GGAACAGACAGTTGTCAGACACCAATGGGTAGGTGCTGGGATTTGAACCCAAGTTCTCTGGAAAAGCAGC
CAGTACTTCTAACCACTGAGCCATCTCTCCCCAAATCCTCCTTGATCTTTGAGACATCTCTCTCTCTTTC
TCTCTGGGACCTAGGGCTTTCCAGTTAGGCTAGGCAGACTGGTCAGCGAGCTCCAGAGATCCACCTGGAT
CTGCCTCCACAGCTCGGGCTGTAAGCACGTGCCAGCGTGCCCCGCTCTTTTTCATGACCATCAGATTTGC
TAGGAAGGCAAGCGCCTTACAGACTAGTCCTTCTTTCCGATCTAAGGAGTGCTCCTTTCTGAGAAAGAAA
CTAAGCACTTGGCAGAGATTGAGAGGAAATTATTTTTGAGTAGAAGATGTAAAGTTTTTTTGAGATTTAC
AGTAACTGTTTCTAAATGTACGTAAGGGAGTAGGTGGGAAGGACGGAGGGCTTATGTATATTATTTCTAA
AACTTCAGGTTTTACTAGTCATAACTAAAACATCTAGAATTTACTGCCTGTAGTTTAACAAAGTTGCCCG
TTTAGAACTTCTACAAATCCTTAAACCCCTCCGCATTCAAACGGTTCAACTCGCTGCTTTGTTTTGTGTT
TGTTTCCTCCCCTTAGCTCCCAAAACATTTAATATTTTGTTAGACGATAGTGGGCTTTCTGACTAATTTT
GAAGGATAATTTACATGACTCTTTTTACATTTTACTCTTTATAATTATAAGGTTTGAGCCACAAGTATTG
TAAAGGAACTGCTTTTTGTTTTCTTATTTTTCTTAAGGAAATACTTTATTTTAATGGCAGTACTTTTGGA
TTTATAAATACTCCATGCATCATAGTGAGTCCTGAATTCAATCCTGAGTTAGTGTGGAAAGGAAATAAGA
GTGATAAGAAAGCGCAGCACAGATCCTGCAGCTCTGTGATAAGCCTTCGCTCTGTGCTGGAGCTGTGGCC
ACCGCGCCCCCCCCCTCCGCTCCCCAACCCCAGTAACTACTGTGTTCTCCCTGACGGGAGGGAAACAGGA
ACAAACACAGTGAGTCCTTAGCACTCAGCCATGGCTTATGTGGGCTGGGGACATGCAGTGGTGCTCGTTT
GCATAGGCCACATGGCTGAGGTGCTCCACGTTTATCTTTTAGAGTATTGATTCTCTGTTGATGCTGTGTC
CTGCTGACAAACATCCCTCACAGGCTGGTGAGCTCTATTTCATCTTAATGCGTGCTGCACAGATAGTAGC
TCAACAAAGAATAGGGTTTGGGGGTTTGGGGTTTTGTTTTAGAGAATGCAAATTTTTATGGGGAAAATTG
AAGCCTTTTTTTTTTTAAGTTTAATAATTTTGTTCAGAGATCTAATAGATATCCAGTGTCAGTTTCCAAA
GACTGAAGTCAGAAATGTCACCTGGATCAAAACCCATGAATTTAGATACTAGAAATAAAATGGAGCCAGG
CACAATACACCGTGCTTAGGAGGCGGAGGCAGGAGGATCGGAAGTTGGAGGCCACCTTAGGCTACATACT
AAGACTTTGTAACAGTGCAGCAAAGTGTGAAATCTAAGTTCTCTCATCCCCGGAGCCCACAGAAAGGGTG
TGACATGGTTCTTCTCACCGTCAGGGGACAACATGGTCCCCAGGTAGTTTTGATTTTTCAAGTAACTTTG
TTTTTAAACCGTAATGTGCCTTATGGAACCTTACCAGATTCTGTCATCTGCTCCGGAGTTTCTGTGAAAG
CTGCCGTCTCCAGCCAGGGTCTGTCGGCTGCCCTGGGCTGGAGGCTGCTTGTAAGTTACAATGAAAATTG
ACCCTAGGTAACGAAATGTTAGTGTAACACGTACAGTATTAATCACGGCAGTGGTTTTTGTTTTTGTCTT
TGTTTTTAATACATAGACCAAGCATCTGAAGTTCAGGTGACGATGATGCTCACCGAGAGCTGCAAGCTTC
GAGGTCTGCACCACAGGTGAGAGCGAGGCTGCGCTTCAGCACCGAGCACAGTGCGACTCCGCACACTTAC
TCACTTTTTAAGAATGTTATGTCTTGTTCAGAACTGAATTCTTTTGGAAATTCTTTATTTGTTTTCCTTC
TGAGTATAAAGGTAAATGCGTGCTCATTGAAAGCAATTTGTAAACAGCAAAGGACGAAGATGGTAGAGAA
CAGGATGTAAGCTATGTAAATAATAAATGTGCACAACTAAGCTTTCTAAAAGAGTCTACGTATCATGTAC
CTGTCTCCCTTTAACAAATATGACAGGGTGATGCATACATTAAAGAAAATTGCTACAAAAATTTATATCT
ATGGAACACCAAGGCCAAAAATAATTAACCATCCATCCTTAGATACAATGAGGTTAATGGTCTGCCATGT
ACTGTTGAGTGTGTATGCATGCCAGTGTGTGTGTGTGTGTATGTGTGTATGTGTATGCATGCCCGTGTGT
GTGTGTGTGTGTGTGTGTGTGTGTGTGTGTAACTACCTGAGATCTTGTATCTAGAAATAGCCATCTATGT
ATTTGTCTGTAGAGATATTTTATAATAAATTTAGTGTGTTTCCAGTGTACTTTTAAAAAGGACAATGTTT
TTTACCTTTAATAAAATTAATTTCTTTATGTAACAATAAAAAAAAAACCTGTTTCTAGGTTTTCATGTTG
GTAACTGTAAATCCCTGTCCCCATTTTAATAGGCCACTTAGTGGTTTACTGAATGACTGTACCACGTTTT
ATTTAATTTATCTCTTATGTATGCTCTTAAAATCATAGTATGATTTCACAAAATGTACCTATTGGAAGAA
CTTTTAACAACTTCTTCTAGACCAGGAATGATTGGGGACAGCAGCATCAGTATCAAGGCAAAGTTTTTGC
ATGTTTGTTGACATAGTTTCTATATAGCCCGGGCTCTTCTGGAACTCACTATTTAGACTCCACTGTCTCT
GCCACCCGCGTGCTGGTATTAAAGGCACTTTCCACCAAGCTGAGCTGGCAGTCGGGGTATGTTTGTTGGT
TTGTTTGAAGCATCATATAGAGAGGTCTAGGAGGTGGTCATGTCTCCGACCCAGAGTTGGAGGGGTACAG
ACAAGTAAATACCTGGAGCTCATTGGGCAGCCTAGCTGAACCATTGTGTTCCAGGCCAGACAGAGACCAA
AGTGGATAACAGTTAAGGAAGGCACATGATGTTGACCTGTGACTTCTATGCACATGCTTAGACTGTACAT
ACTTATATATTCCTACATGTATATACACCACACACAAGTATTGTGTAGACTCTGCTTAGTTTGTATGACT
ACTGAGAGTTTCACATATCTGAAAAGTGAACCCTGACTCTGTGTGTTAGAAGGCTCTGTGGGCTTCTGGG
AAGGCCTTGTGATGTGTGCAGAGGGCACCATTGGCTTCCATTTGGACTTGGGCTGTTACTGAGCTCTGAT
AAAAGTATGAATACACAGAAATTCCTTCTTTGCATTTTGTTTTTACTTTGGGTTTTGATGGCCTAAAAAG
ATGACAGCCACATGAAAATGAGTTCAGTGGTAATGAAAACGAGACAGATATTTGGCATTGTGGTGGTGTA
AAGAAAATAAGCCAAGAGGAGAAGACAACTGTTTGGACTGAGATTAAACACAGCATAATTCTTGGCTTCT
GATCAGCTGAGATGAAGGTGACATTAAAGTCTCTAAGTCTCAGAAGCAGGGGGAAGAGAAAGCGCGTTGT
TAATAAGCTCTTTTAGTCTCAGATCCCAAGACGGAGGTTTGCATTAGGTTCATTATTTAATGTAGATTCT
GATAATATAAAAGTAAATATTCATTTTCTAAAACTCCATTGAAAAACGAGGCTTTTGAAGAAAAAGTTGA
ACGCAATATTGAGAAACAGTGAGGGTATTCCTTGGAAGAGTTGAGTAATATAACTCAAGAATGTTGCCTT
CAATTGGCCAGATTCAGCCCCAAGAGTAGAAGTCCTCACTTGTTGGGAAATAGACAGCACCCCCCCCCCC
CGGCTATATTTGCTTTAAGAAAGGAAGGAAGGAAAGGAAGAAAGAAAAAGAGAGGGAGGGAAGAAAGAAA
CTAAGTTATATTTGCTAAAAGCCAGAAACACGTCTGGAGCTTTTAGACCCTTTTTAACAGTGTTGTGAGT
GAGTGCTTGATGCTGACGCAGACTCTAGAGGAAGCACCTTCTGGCTGATCACTTATGCTCCCGGCCAGTT
CCCTGACCCTGTGAAGCTGTAACCGGCAGGACAGAAGCACGTGGCAGGAGTTAATGCAGACAAAGCCACA
GACTTTTCCCAAGTAGGAGCTTTAGATCTACCCTAGCCCTGCGCATCCCTTGGCTGAGCAACCTTCCAGG
CAGGCACTTACGTTCTCTCATGCAGCTTTAATCTGCACTGCTTCACAGCATCCCTCCATGATGGGCTAGC
AGTCTCAGACACCCCACAGAAGGAGAGGCGAAGCAAGTTGGTCAGAGTTCATGTCATCTCGGCTGTGAAG
TGACGTTAAGATCCCTAGTTGTGGCAGTAAAGCTGCTGGTATGCTCTATTCCCTGTAGTCCTCCATCTGA
CCACACGCAGTTAATGGAATACTCAAGGCTGTGCGATTGCTTGCTTCAATTTCATTCACTAACTGCCCGT
TTGTACTTAGGATTCAGGTTGGTGTAGAATAATAATAATAATAAAAAAGGTCATCTGGTTTTTTGGTTTT
TTTAAAAAAGATTTATTTATTTATTTCATGTGTATGAGTACACTGTAGGTGTCTTCAGACACTCCAGAAG
AGGGCATCAGATCTCATTACAGATGGTTGTGAGCCACCATGTGGTTGCTGGGACTTGAACTCAGGACCTC
TGGAAGAGCAGTCAGTGCTCTTAACCGCTGAGCCTTCTCTCTAGCTCAAGAGTAATCTTAAGATCAGTAC
CATAGAGTTTATTGTGCAGGCTCAGTGCACTCAGCAAGAAATGTCACCTGTTTTCAGGTGTCACCTTTTC
ATTGGTTGTAGTCTGTGAGTCAGAAGGTTTGCTTTGTGTTTTTACATTCTGTGATACAGGTTAAGAATCC
CTGACACGTTTGAAAGCATCTTGACATTCTAATTTTTTTGTTAGCTTTGGTTTTTCTACTGGAACTTGTA
AGTTTTCTCATATTGAAATGCTTGCATAAATTAAAAATAAACTGCTTTCATTACTTCATGTCTGAAAACC
CCTAAGCCGTTTTTTGTTTTTTTGTGTTTTTTTGTGGTGTTTTGGTTTTTTTGTTTTGTTTTGTTTTGTT
TTTGTGGTTTGAAGTCCCCATTGGCCCAGAATGCCCAGTGATGGCTGGCCCTTGTTAATGCATAATGACA
GGTAGCTTTAGGGTTGTAAGCATGCTCAGCATGGGAGGCAGTTAGGCCCAGCCCCGAAGCCCTGCAGCCC
CACCTCGCCTCCGCCTTCTCACCTCTCCTTTACCTCCATCTGTCCATTCTTCGATATCTCAGACCTTTCT
GTTTGTCCTCCTTTCCTTTGTAAAATGGGCAGTGAAGAACAGTCATTTATTCTTGGAAACATTTTCATAA
AGCTTTTTACTATTTCCAGAGTTTTTCCAGGTTTGGTTTTTGTGTATATGGGTGTTTTGCATGTATGTGT
GTATGTCTGTGCACCACATTATGCGGGGCCTGAGGGGGCAGACGAGGCATTGGGTCCCCTGAGACTGTAC
TTAGAGTCAATTGTGAGGCACCATATGAGTTCTGGGAATCAAACCTGGTCCCTCTGCAAGAGCAGCCAGT
GTTCTTAACTACTTAACCACAAGAGTAAACATTTTGGTTTTATTTATCTTATTTGTTGTGTCTGGACAAA
TGCCATTAATTCTGCTAAGCAATTTCAGTCATGGGTGAAGGGACAGAGATAATCCTTTAGGGTTAAGGAA
GTGGCCAGCTCACCTGCTTATCCTGAAGCAGGGTTAGCCCTGATTGATCCTCTAGGTGAAGCTCGAATTG
GAGTGAGTGTGTGTGTCTGTGTGTGTGTCTGTGTGTGTGTCTGTGTGTGTGCTTCTGGAAAGGAGGGGCT
CTGCAGTCCTTCCTGCATTCTCCTTTATCCTGGGAAGGAAATGTCCAGCAGCCCAGGGGAAAGCACAGGG
GCGTAGAATCTGATGACACCTAAATTTCCTTCTCTTTATACTTGATCTTTGTCCATAGGTTCCTAACCAC
TCTTCAGCTGTAACGCTAAAGTCTTTGATGTGTCCGTCCAGTGCCTCTTAGAGACTGATGCTTCTGGCAA
TGGGGACACTCTTAGGACACAAAGGCCAAGCAGAAACTTACTGTCATCTCACAAGTCAGAGACAGCGCCT
CTTGGTACATTTCTGCAGCTTCCCCTTTGTCTTTGTGGTTGTCCTGTGAATGTGTGGGTTGTGCCTCAGT
TTCTTTGAGTTCTTTTCATTTTTTTAAAGATATTTATTTATTATATGTAAGTACACTGTAGCTGTCTTCA
GACACCCCAGAAGAGGGCGTCAGATCTCATTATGGATGGTTGTGAGCCACCATGTGGTTGCTGGGATTTG
AACTCAGGACCTCTGGAAGAACAGTCATTGCTCTTAACCGCTCAGCCATCTCTCCAGCCCGCTCTTTTCA
TTTTTTGATCGTTCAGTTGTAAGGTGTTACTTTGTCATTCTTGGGCAACTTTTCACTGTTGATATCCTAT
TGACTTCTCTTGTTAATGCACCTTTTTATGTTTCATATTAATCTTTTAAAAAACTTTTTGACATTGGTGT
TCTGCCTCCAAGTGTGTCTGTGTGAGGGTGTCAGATTCCCTAGAACTGGGGTCACAGACTGCCATGTGGG
TGCTAGAACTTGAACTTGGGTCCTTTAGAAGAGCAGCCAGTGCTCTTAACCCCCAAGCCACCTCTCTAGC
CCCCATGTACTCATTCTTTATGAAGCCATAAAGAACTAATAAATAGTGCTTTTAGGGTATTGTTATGTAT
GTGTTTCAAAAATGCATTAAATGTAACCACACAGGAGGTGGCACTGAGTATGTAAATTGAGTCTATGTTA
TTTCTGGCTTAATAGAGAGGAGGTAATTATTTAAATACTTACAACCTTGGGGAAAGTGAGTTATTTTCAT
ATATCAGCTGGAGTGTCATTACACAGGAGGATGGGACGTGTGTGTGTGTGTGTGTGTGTGTGTGTGTGTA
TATGAGTGCCATGAGAATTAAAGTATGCAAGATTGTTCAGTGCATTCTTATTTATTGTGTACATAAGAAT
TGTTTGAAATTCACATGTTACAAGTTTGAAAACTAAGGCCAAGTGGGGGAGGGGAGCAGGGCCCCCATGC
ACTCCCATATCCACTCTCACAAGCATGCACAAACACACACACACACACACACACACACAATCACTCAGAT
AGTGAATCTTGTTTACATACACACACACACACACACACACACAGAGTGAATCTTGTTTTCAAAACTCAGG
TTCCAAATTCTACTGCAAGCATTTGACTTTGTGACAGTTTCAACCTGTTGGAGACTTTTGATGGTCCCGT
TTTATTTGCAGCGGGGATGGGGCCTTTTTGATTTCCAGCGTGACAGTAAAGTCGGTGGGACAGCGTGATG
ATGAGCTCCGGGCCAAGCTGGCCAAGCAGCCATCAGCTGTCTGCGCCTGGCCTTTAGGGTGGGGATTAAC
ATGGCCGTCAGCAAATCTAATACAAAGGAATTCATTCTGGGTAGTCATTTTTATTCTACAAAGAAAGCTC
ATACTTATGAGGACTTTTCACATCTGGTTTAAGAAGTAGGCCATTTGTAAATAAATATTTTAATGATAAT
CTTTTTAAGTGTGTTTGGAATTTCATTTTGCTTTCGATTATCTTTATTACTTTTTATTTAAGATAAATCA
GTAAGGTTTTAGATTTCACTTGCATTTTTCCAAATCTCTTATTATTTTCTAGAAACCTCCTTCCTATTAC
TCATGTGTGCATAGAAGAAGGAGAAAAGCCCATGGTGGTATTGCCATACATGAATTGGGGGAATCTTAAA
TTATTTCTTCGGCAGTGCAAATTAGTAGAAGCCAATAATCCACAGGTGAGAAAATTTCCCTAAATATGAA
AAATTCCTAATGGTTTTGCAAGTAGATATTAATAACCACACACATAGTGTCTAAAGATTAAAAAAAAAAA
AGACCTCAGAATGAACCAGGAGATATTTATAAGCTGTCGATTTAGAGGACCGTGAAGATGGCCTGTGGCT
TCCCAGTGGAGCACTCTTGGCCCATGCTGTTTCTCATTGTTCGCCACACCTTCATTCATAAATCACCTCT
TATGTCCTATGACAGCATCTCAAAGTCTCTTCCGGAGTAACTTCTGAAGACGGCCTAGGATTTCCTTTCT
TGGAATAGAAATCATAGCAGAGCGGTCTAACCATATGGTGCCAAGGGCACTGAAGGTTGGAGGTCAGGAT
CGTGGGGGATAATCAGGAATATCCTTAATGTGCCTGACTGTGAGACGCCACGGTTTAAGTTTCTCTCTTG
TCACTACCGACGTGCTGTGGTAGTGATCAGAGAGTCTGTTGGGTGCTTTGCTGTCTGCTATATTTTCTGT
TCTTGTTAAATTGATTTTAAGACTTAAAATAGCATACTTCTTGTAGTTAATAAGAAGAACAAACTCAGAT
CTGCACTGAGTATTTGACTCTTAATGTATATGATATGTCGTTTAAGCTAAAAGACTAAAGCTCTGGTTAG
AACCATGTAGTTAGAGGTCGTGGTCTGTGGGTGTCACCAGGAGAGGAACTTCCCTGCCCAGCCTGCAGTG
CCGGCGGGCATCTCGAGGAGCGTGTCTGCAGAGAGACTGGTTTTTGGTAGAATGCTGCCCCCACCCCAGG
GTGGTGGTGGTGGTGGTGGTGATAATCTGATAAGACCTACTACTACGAAAGAAAAGAGCCAGTCACTAGA
GAGATACATGAAAATGATAAGTGTCATGGATAAAGTCTCATCGGTGGTAAATCCTGCTACCCAACAGGGG
CTTTTGTAATGGATGAACAGTATTCCGTGCTGCCAGAGCACATCTTGGTTGAGAAATAGGTTTTCAAAAT
CTCAGTAAGTAGGTCCAAGGGAGTGTTATGGGGTGGGACTGTAGACTCAGGTAATCAGATCCCAAACTAG
TTCTGCAGTATCTACAGACTGTGTCATTTCTTTTCCTCTGTTACCACAGGCAATTTCCCAGCAAGATCTG
GTCCATATGGCTATTCAGATTGCCTGCGGGATGAGCTACCTGGCGAGGAGAGAAGTGATCCATAGAGACC
TGGCTGCTAGGAACTGTGTGTAGGTGTCCGCCGCTCCCTGGGCTTGCCCTCTGTCTGGGAGTCCCGGGGC
ACACAGACTGTTTGTGGTGATGCTGCATCTTAACACCCTGCTACCTTCTATGTTCATGTCTCTCTCTTCA
GCATCGACGACACTCTTCAAGTCAAGATCACAGACAATGCCCTTTCCAGAGACTTGTTTCCTATGGACTA
CCACTGCCTAGGGGACAACGAGAACAGGCCAGTGAGATGGATGGCTCTGGAAAGTCTGGTTAATAATGAG
TTCTCTAGTGCTAGTGACGTGGTAAGTACTGGAGCCCAGGTCAACAGCATGTAGAGAAAAGCTTTCTGTT
TCACTACAGTCAGTGTTTTGTTCTTTTAAAAATCTTGGTGCTGTTCCTTGAAATTTTCATCCAGAGAGCC
TGTGTACTTAGCTCCCTCTTGGCCCACTTAAAGCAGAGCACCGGCCCAGATCCTGTGTTCTTGCTCACAC
CAGCTGAGGCAGCACCATTCCCACTGAGGGGCGCTGAGGTTAGGGAGCGGGCGGCCACTATCATCTCCTT
GCACAAGTCTCAGGATGCCTGAGTGGCTACTAAAGGCAGAATGGTTGCTTTCTGAGCATGCTCTCTTCTC
AGTCTGCAGCCTCTCTCATCTGCAGTCCTGACACTGTTCCTGTTAGGTGTCAGCGTAGTTCTTTCTCCTC
TTCCAGAGCACAGAGTTGAGCCCTTCTGCTCAGTGATGTGCTTCCAGAAGCCGCATCTACTCGTCCGTTG
TAAGGTTCTGTCCCGCTACTTTTTATTCTAGTCTAGATCATAATCAGCCCAGACTTGGAGGCGCGGGCCT
ATCGTTCCAGACCCACCTAGGGTACATAGTGACTGTGTATCAAGAAAGAATGAAAAAGTAATATGATTTT
TGTTTGTTTTGTTTTTATCTCTTGACTGTTGCACACTGTAGCTGTGTATTTGGAAATTAGATTAAAAAGT
TTGGCTATTAAGGTCCCTAAGTCATCATTTGACAATACTACATTACTTGTAAAATAATTGGGATTTGATC
TCATGTTTCAACATCTAAGACCTGGGTTGATTGTGTTTGGGCGTCTGGCTTATTTTATTATTCAGAGCTG
CCTTTCCATTTCGGCTTGTATGCTTCACACGTCTCCCTTTAATAGTGGGCTACTGACACAGCTCCTACTC
GTTACTTACATTGATTAGTGCACATGCTTTCCCCCCGTGTATTGATTTTGCTGGCTCTATAACAAATTGC
TCAGGGCTCAGATATAAATGGACTAGACATTATAAGTGTATATCATGCCTTATAGTTTTCATAATTGTTA
TAATTGTATTCAATTTATATCTGAGAGGAAAAAGAGCCTTTTGTTGGACTAAAAAGGGGAGTATCGTTGA
GGTTTGGCCTTTTGCTATGTACTGTGATGCTAAATATCAGCTCCCAAGGCCTGCTGGACCTCAAGGACTA
GAGAATCTACACAGAGAACCGACTGACAACGCTGTGGCCTTGCCCCCAAGTCATCCCTGATTGGTGAATA
AGATGCCTACAGCCTGTAGCTGGACAGGATTGAGATGGGCAGGGCTCGGGGTTCCCAAGCTTGGGGTTGA
GAAAAGGGAGGAGGAGGGAAGAAGGAAGGAGCCATGAATTAGGTGAGTCAGTCCTAAAAGGACAACCATG
AGGACTGGCAGTGAAGTTACGGGCAGCCCAGATGGAGCATGGCAAGTGATAACTCCGGGTTACTAATGGG
GAAATAGATTCTAGTAGTGTAGAGGATAGATAGCTGCCCAACTCTAGTGCTGATTAAGTCTTGTTATGAT
GATAAAAGTTTCGTGTATTTTATCTGGGAACTGAATGATCAAAGGTGGGGTAGAAACCCTGACTTGAGAT
TAAATAATTACAGCAATATTTTATTGCTAAATTCCTTTGGCATAACAATAGTACTTGGTTTTCTTATAGG
TCTGTGGTCTATTTAAGCTAGGTTCTTGGCCACCCAAGCAGTGTCAGGCATGGGTTTTGTCTCATGGAGT
GGGCCTTAAATCCTGTCACTAGAGGTGTCACTTGGTGGAGAGATGTCTAGTTGGAGGATTCTCTCCTCCA
TTATTTGGTGACTCCATTTAGATTTCTTTCATATATGTATGTATTTTAAGAAATCTCTGTTGTAGGTTTC
CATGTGACTCCTCAAATGGCCCTCCTTCATTTTGTTTTGATCATTGTGAGTCCAGTACTGGACATCTTAA
CATAATTCTCTAAATTTAGAATACCAACTCCCAGTTCAACAAAGTTCTCCCTCATTACAGCCCATGCTGG
TCTTGGGCATGAAATCTTGCCTCAGCCTTCTAAGTGCTAGGATTACAGGCATGTGTTTTGTCTTATTCCA
AGTTGAATTGAGCTTAGCATATTATTGTAAGAATGTCCCTTCATTAAATTTAACGTGGCTAACTCCTAAA
AATGATCAGATTTTTCCCTTAAATTCTTCCAGACGCATTTCATATGCATAAATACTCCCTATAAGTATCT
TTTGCCTTGAATACTTGGGACCAAAAGAAATATTTATGTCTTTTCCTGTGACTATGATAGGCTGTTAGTT
TAAAATTCTGCCATCAAGTTGGGGCATTGCATATTGTTTTAAATTCTTAAAGGTTATTGTTACATTCTAA
CATTGGGGTTAAAAATGCTAACATGTTGAACACATGAACACATGTTGACTTTGCTCTCTTTGGGTGGAGG
TGGGATTCGTATTCATTCTCTCTTCTCTCTCTCTTTTCTCTCTTTTCTCTCTCTCTTCTCTCTCTCTCTC
TCTTTTTCTCTCCCTCTCTCTCTCTCTCTCTCTCTCTCTCTCTCCCTCTCCCTCTCTCTCTCTCTCTCTC
TCTCTCTCTCTCTCTCTCTCTCTCTCTTCTAGGCACAGTTTCTCTGTTTAACCCTGGCTGTCCTTGAACT
TGCCCTGTAGACCAGGCTGGTCTTGAACTTAAGGAGCCAAGCACCTCTATCTCCTGAGCGCTAGGAGGGG
TTGTAAAGGTTGTACACTGAGGTTGAAACTCAGTGTCATTTCTTAAGGGTGCTGAACAGTTTTCACCATC
AGGTACTAAGAACTGTAACCCTGCTCCCACTGAATCTGCACATGGGAAGTGCACACTTGCAGGTGTGTAA
GTGCATACTTGCAGGTGTAGCTCAGGACCCTTTTTTCTCCTTGAAAGAACCTTATAGTTCTGCAGTTTTG
TAGTTAAGGAGTCTTCATGATTTGCTTAGAAACACACTGGTTGGTTGTTTTGTCATAATAGTAACGCATT
GCCACTTTGGGCTTTGTTTTTGTAATTCTATCTGGAAACATCCAATATGTTGGTACATTAAGATTCTAAT
AACGTTAACATCAGCTTTTAACTGTCAGCTTAGGAAGTACACACAACCAGATCCAGCTAGTTACGAATGT
CTAGTATGCCAAGATGGGACCTCACATAGGCACTCTGAAGACGTCTGGGTATTGTTCTTGTTCTATGTTT
TTGCCAAATGTAGGCACAACTTCAGATGTCTGTTACGACTTTTTTCCCTTTATTTGAAAAAATACTTTTT
GATTGATGCCTCCTTTGAATTATTTAGATAATTATAGTAGCTAACACAGTTCAGCTGTCAAGAGTCTGCC
ACCGATTGAGTGGTGAATGCAATCAGGAAAGCACAGTTGAGAGTTTCATGTTGTTTTTCCTCGTCTTTGT
GGTACTACATTGTGTTTAAAAAGCATTTTCTTGGGAAAAACAAATCAAAGAAATGACTTCCATGCTAATC
TTATCAGCATGGTTTATGGCACTCGGGGGATCCTTTTTAAGGCATGTGAATAGTTACTAGGCCATAGAGC
AAATCAAATGCCCCCCATGTGCCTGCTGTTTCTTTCTAGACACCGGTGGACATTGTCCTGGGAGCAGAGT
CTGCAGTCATTCAGAGCCTGCATCTGCAGCATGCTCCATCTTGCTCCAGTCCGCCAAGTGCTCTAGCGGC
CATCGCCAGATACTGAGTAGGCTCAGTCAGGAGACTCATGTACAGCGGGTGGTGTCTAAGGGAAAGCTGG
AGTTTCTGAAAGACGCTTCTTATTTTCAAAAGCAGAGAACTCTTTCTGTCTCATATTTTCCATTACTAAT
TTTAGGTAGGGCTTAGAAGATCCTCAGTAGTTAGTTGTGTGTTTCCTTCATGGTTGCAGATTTTATACAC
CAACAATCTTAGACCTATTATTTCAGTATCAAATTCACATTAAGAGAATTAGTGTAGGGACAGAAGAACA
ATGTAGACATGAAAGGATCAGTTTAGACCATGGTTTAGGAAATAGAGATTTAAGATTTAATGTTCTTGAT
GTTAGGGTCCTGCTTCATTGTATTGCTCTAGCGTTGGTTGTCCTGGCGTTTCTTAAGAACGAGCAGTACA
GGGATCACTCTAAGAACAGTTGCTGTATTTCCAGACGTGGTCGACTCCCCTGGCAGTGTTGTCTGCAGTA
CTGCTCTCTCAGGATCTCACGCGTCTCTGAGCATCTCTCCTCTGGAGCGCTTTGGAGACGTGCTCCTACT
CTCTGCTCCTACTGTCTCCCTTAGAAGACCCCAGGATTCCGATGAAGCCCTGTGCGAGTGTCTGCTGGCG
GAGAAAGTGGCGTGAGCAGCCAGTTCTGTGATAGCCGTCGGTTGCGTGTAGGTCTGCAGGTGTCTAAGGA
TTGGTCACACCAGCAGCTGCAGATGCTGGCTTTTGAGGAAGGCGGATAGACCCAGTAGCTCAGGACACAG
GCTGCATTCCTCCAGCTCTGCAGCCTGGGGAGTCCAGCATGAGGGCACTGATCAGTGTCCTGCTTTCTGC
TATTTCTTCACATGGCAGAGGGAAGAGTGGGGTTTGCTGAGGTCTTTATAAGACTTTATAAGCTCCCATT
CCTGGGAGCTCCACCTTTATGGTGTTAACTCCTCCCATAGCCCCACCCGCTAGCTTTATCACACTGGGCA
TCGGGAGTTCAAAATGTACACATGGTGGAGGCATGGTGGAGTCTCTCAAGTTCTGCTGCTGGGCCTCCTA
ATTCACACCCCTCATGTAGTAGATCGCTTTCATTCCTTCCTGACGTTCCTTCCTCATTTGCCTAAAATCA
GTCTCATCAGATGTGAGTGAGCCTCAAGGTATGATTTATACTCAGGTGAATTTGTTTTCAGCTCTAAGTT
TAGGAAAGGAAGCAAGTTACCGACTTCCAGAAACGCAGCGGTGGGACGGGTTCAAGTTGGTGCATCTGTC
TCCAAAAGCGTAAAACAGGAAGGGAAAGAAACCCAACAAACACAGTGCACCTTAGACTCAAGCAGGCCTC
TAGCTCAGTGCCTTGCCATCCAGGACCCTGGTCCCTTCATCAACAGCTGCCCATGAAAGTTCATTTGTGG
CTCTCCACTGGGGTCCCAGCCAGGCAGGGCAGAGCATGAGAGTATCCCGTCACACGTTTCCAGTGAGTGC
TAAGGTTTCGTCTCAGTTAACTGGCTGGGGCGATTGCTCTGCTGAAGCCTGTCTGGCATTTTAAATTCCC
AGAGGACTCACACAGAAGCTGCCCCATTCAGTGACCTCACCCTCATACGTTAAGATGAAAACAATTGTGT
ACTTTCTGACAAACCATGCCAGTATATATTGTTCATTAAAAAAAAAAAAAAAAACCTAGTTTTTGTCCTT
CATTTTAAATAACAAGGTCAGAAGCTATGTCTCTTGGCGCATGTCTGTAGTCTCAGCACTTTAGGAAGTT
GGGGAAGGGAAGTTGTTGGAGTCTCAGGCCAGCCTGGGTTACAGAGAGAGACCTGCCCCCACCAGTGCCA
CCACCACCACTCGCAAAATAGAGGCTAGCATTCATCTGCACCTGTTACTTATACCTCCTTCTGTCAAAGA
TGGCCTGATGCTCACTGGTGAAAGGACAGCAGATTCTGAAAGGCTTTGCTATTCTGGCAATGCTGCCCCT
GAGCACATGGATAGTGGGGGAACTGTGTTCAACCCCCACCCCCATTCTCCAAATGACAGGTGGGAAATTC
TAACCCCTGAAACTAAGACTATAACTTTAAGAACACCTAGGACTTCCAGTGACCAGTAGAAGCTGGAATT
AGGAAGGAAGGGATCACCTTTTAGAGCAATATTTAATGTCATTCCATTATGGATTTTTTGTTTTGTTTTG
TTTTATAGATTCTGTTAAAAATGTGGCTATTGTTCACTAATATAGGTTGAATAGCCTTCGTCCAAAATGC
TTGGGTTTTTTTCCCTAGACTTAATACATACATACATACATACATACATACATACATATGCTGTATAAAT
GGGACTCAAATCTAAACACAGGTCTGTTTTGTGTAATATTTTTAATGATTTTGTGCATGAAACAGTTTCA
TGGCATGGAAATTTCTTTATTGTAGTGTCAGCTCTATAACTTAGATTTTGGTGTGTTTTGCATTAGGGAT
ATTCAACCTGTGAAAGGCTTTTTTAAAAACAATCTGTGTATTTTACACAGTGTCTCCAGATTTTGTTCGT
ACTGAAACAGTAACCTCCAGCTTTGTGCTCTGGGTGTGGATGTGCTGTGTGCCCCACCCAGCTCTGCCCT
TTCACTCACTGAGATGTGTGCTTGGCCAGCCATAGGTTTCACCTGCTTCCTCGCCTGGTAACCTAAGCTG
CCAGCGTGAGGTTCAAGGGTTTGTTGTGAAAACTGAATGTGATTGGAGGCTCATCGCAAAGCACTGGTGA
GAACCCTTTAGCTCCTACACAGGTGCTGCCCCCTCTCCCCAGCGTTGACGTGAACTCAGGTTCACAGGTG
GCAGCAGGTTGTCCCCACCGTGCCCTGGACAGCAGCATAGCCGCAGCTCTGGGTGGTGGTGGAGCCTGCC
TTCCACTGCTCGTTCTTCTCATGAAGGGAAGGCCCGAGTCAGCCGGCCGGGCCTTGCAGTGCCTCTGCTT
GGGCTCTGCGCTTACTGTTCATCCCCTTACTGGTTTTAGGTAACTCTGACTTTTACTCAGCTGGACAGTA
TCTTTTCAGGGTGTTCCTAAAAGTTGTAAATGGTGACAACAACACATTGTCTCTCTGTGCATGCTAGGGT
TCCATCCCTCGCTCTGCAAAATAAGCAGATCAGAAACCCATGACTGCTTCTATGTTATGTAATCTGCATG
TCTCGCTTACTGAGGGAATCTTCTGTCCATCTAGGTTGTTCTCCATTTCCCACCGTAATTCGTTAACTTA
GGGGTAGCATGGTGAATAGCCAGTGATAGAAGTGGTTTCTTTGAACGGACCTAGTTACTTAGACCAAATG
ATACAAATGTTAACATTGAAGAATTAATGCATTTTAATGCTTTCTTTCCTACACCAGCTGGAGAAACTAG
CCAGTATGGTAGCTTTTGTTTTATGAGCAGTACTCACTTTATTTAGGAGTTTGTTCAGTTGCTATTTGTG
ACCTGTCTGTAGTCACCAGCGCCCAGTTTTAGAATGTTCCCCTCATTGTGCTGCTGTCCTCTCCTAGTGG
GCCTTTGGAGTGACGCTGTGGGAGCTCATGACTCTGGGCCAGACGCCCTACGTGGACATCGACCCCTTTG
AGATGGCCGCTTACCTGAAAGATGGTTACCGAATAGCCCAGCCAATCAACTGCCCTGATGAACTGTGAGT
TTTTTGGATCTCAGACATGAGCCTTGGTGCATTTACTATGTTACTATAAAGCAGGAAAGCACGTAAGATA
CTCCTTCAGAGTAAAGAAAAAAGTAGAGGACAGAACTCAGTGCTGACCTCTGCTCTCCACAGACATGCAC
AGACACATGCATACAACCACACAGACATGCACATAGACTACATGACAGTAAACACAGACAGAGACAGACA
CCTTTCTCATCCTGGCCTTGCCTTCCTTCCTTGCCTCCATCATGGCGGTGGGGTACACAGGGCCTGGTTG
GGATGGTGGCACAGCAATGTGTTGTCTACAGAGGCCCTAAAAGGTTTCCCTGCCAGTTTTAGATAGTAGC
TTTGCTGTAGATCCAGGAGTCTTAATTTCAGGTAACACTTCTTCCTAGTTCTTCCTCAACAGCGTCCAGG
ACAACGCAGCCTGTTATTCTGACTGTTCTGTGTATTTACACAAAGCTCTGAACTTTCTGAGTATTGTATT
CTCATAAAGAACTGAAGATGGTTGTGTGTAATCCAAAGAACTTAACTTCCTTTAATCCTGAAGCTGCTAA
CAGTACAGCTAAGCGATTGCTCATGGCTGTCCTCCCCAGCAGCCCATTAAACAGAGCACATAACTGGCAG
GACGCTGACGGAGGCCTGACACGTTTCCATGTCACTGCAGGTTTGCTGTGATGGCCTGTTGCTGGGCCTT
GGACCCTGAGGAGAGGCCTAAGTTCCAGCAGCTGGTCCAGTGCCTCACAGAGTTCCACGCTGCCCTGGGA
GCCTACGTCTGACTTCTCTCCCAGCTCCGCCACTCAGAAGAAAGTGCCTGTCTGTCACGGATGCCCCTCG
TGCAGCGCAGTGCCTGCAGGGGCACACTGTCTCCAGATCACCCAGCCTTAGCAGTGCTTCCAAACCTCAG
CTTTTAACGATGACGTAATAACGCAGAGTGTTTTCTAGATACCACTTTTACTAGGTTGAACCAAAAGGGG
TTTTGTACATTTTTTGGCCAAAAATTTTTTTAAAATATAGTGACTTTGGACTAGGGGGTGCATTCTTTTA
AAAAATGAATAAACAGTTTTAAAAATTATTTAGACACAGATATTTGGAATAGCTGTCTTAGTGCCAACTG
CTTTTATTCTTAATGTTTTACTTCATTGAAGTAATGTAGGTGACTCACCTTTAAAGCTATTTTAATATTT
TGGTCACTACTTTTGGGAGTGGTTTGTCTTCAAAATGCGGTACTTTTCTTAGGAAAAACAACATTACGAA
GTTGTCTGGTTTGCCTTATACGAAGAAAGATAGTATATTAGGAAAATTGAAAAAGTAAAAAGAAAAAGAA
ATTACAAAGTAAAAATAAAAAAAAAAAAACATATCGCCCCCGCATGGAAAATCGCTTGTTTAGTGACCAT
GGCTCTGCTCACATTTGGGGAAGCAGGTCTGAGTTCACATCTCAGTCAGCTTTCCTGTGAGCTGGAAGAA
GGCTGGGGCAGGCTTGTGGAAAACAGTGGTGACCTTATTTATTTTTGCTCTCCGGGGAAGGAGAGGACAC
AGTTCCTGACAGTGACCCCTCTCTACTGCTGCGGTTCCCTCTGTGCACGCCTGGGCGGAGCTACGCACAG
AGCCGGTGCTTCTGTTGTTGTGGGTTTGGGGGGTTTTAGTTTTGGTTTTTGGTTTAAATTGGTAATTTTT
ATGCTTATCTTCAAGGCTGGCTTAAGTATAAATTTGTTTTTTTAAAACACTTGAAAAATTAAAGGACTCA
TTTTATATGCTGGTATTCAAAATACTGCTCACAGTGTGTGACTGTTGGTCGTGTCCTGAAAGTCTTGGAG
TCACATTTTATCCCCTGAGAGTTTGACTCAGGTTCCAAAACCCTAACAGAAGAACAGCTGACTCTGCTCA
GCCGCCAGGGCCTGTTAAGAAGTTGGAGTTACTCCTACAAGGACTTATCATGGAGAGAGTCCACAAATAC
ATGTATGTAGTTACACACAGGTTTGGTCTACCTAAGGGATTGTCACCTGGGCCTGTGAAGTGACTGGGCC
TGCAGCTACTGCCTCTGTGTCTGTCCTCTCAGGGATGTGAGGGAGAACATGGGTCTGTTCTATTAAAATA
AGTCTTGTGAATAAAGAGAGTATCTGAA

An exemplary DNA sequence of the Ryk gene in human is provided below.

(SEQ ID NO: 19)

CGGCTCGGGGCTGTGAGCGGCTCGGGGCCGGGGGTGGGCGGCGGTGCGGC
GGGCGGCCGACGCTCCTCTTCGGCGGCGGCGGCGGCGGCCATGCGTGGGG
CGGCGCGGCTGGGGCGGCCGGGCCGGAGTTGCCTCCCGGGGGCCCGCGGC
CTGAGGGCCCCGCCGCCGCCGCCGCTGCTGCTTCTGCTTGCGCTGTTGCC
GCTGCTGCCCGCGCCTGGCGCTGCCGCCGCCCCCGCCCCGCGGCCCCCGG
AGCTGCAGTCGGCTTCCGCGGGGCCCAGCGTGAGTCTCTACCTGAGCGAG
GACGAGGTGCGCCGGCTGATCGGTCTTGATGCAGAACTTTATTATGTGAG
AAATGACCTTATTAGTCACTACGCTCTATCCTTTAGTCTGTTAGTACCCA
GTGAGACAAATTTCCTGCACTTCACCTGGCATGCGAAGTCCAAGGTTGAA
TATAAGCTGGGATTCCAAGTGGACAATGTTTTGGCAATGGATATGCCCCA
GGTCAACATTTCTGTTCAGGGGGAAGTTCCACGCACTTTATCAGTGTTTC
GGGTAGAGCTTTCCTGTACTGGCAAAGTAGATTCTGAAGTTATGATACTA
ATGCAGCTCAACTTGACAGTAAATTCTTCAAAAAATTTTACCGTCTTAAA
TTTTAAACGAAGGAAAATGTGCTACAAAAAACTTGAAGAAGTAAAAACTT
CAGCCTTGGACAAAAACACTAGCAGAACTATTTATGATCCTGTACATGCA
GCTCCAACCACTTCTACGCGTGTGTTTTATATTAGTGTAGGGGTTTGTTG
TGCAGTAATATTTCTCGTAGCAATAATATTAGCTGTTTTGCACCTTCATA
GTATGAAAAGGATTGAACTGGATGACAGCATTAGTGCCAGCAGTAGTTCC
CAAGGGCTGTCTCAGCCATCCACCCAGACGACTCAGTATCTGAGAGCAGA
CACGCCCAACAATGCAACTCCTATCACCAGCTCCTTAGGTTATCCTACCT
TGCGGATAGAGAAGAACGACTTGAGAAGTGTCACTCTTTTGGAGGCCAAA
GGCAAGGTGAAGGATATAGCAATATCCAGAGAGAGGATAACTCTAAAAGA
TGTACTCCAAGAAGGTACTTTTGGGCGTATTTTCCATGGGATTTTAATAG
ATGAAAAAGATCCAAATAAAGAAAAACAAGCATTTGTCAAAACAGTTAAA
GATCAAGCTTCTGAAATTCAGGTGACAATGATGCTCACTGAAAGTTGTAA
GCTGCGAGGTCTTCATCACAGAAATCTTCTTCCTATTACTCATGTGTGTA
TAGAAGAAGGAGAAAAGCCCATGGTGATATTGCCTTACATGAATTGGGGG
AATCTTAAATTGTTTTTACGACAGTGCAAGTTAGTAGAGGCCAATAATCC
ACAGGCAATTTCTCAGCAAGACCTGGTACACATGGCTATTCAGATTGCCT
GTGGAATGAGCTACCTGGCCAGAAGGGAAGTCATCCACAAAGACCTGGCT
GCCAGGAACTGTGTCATTGATGACACACTTCAAGTTAAGATCACAGACAA
TGCCCTCTCCAGAGACTTGTTCCCCATGGACTATCACTGTCTGGGGGACA
ATGAAAACAGGCCAGTTCGTTGGATGGCTCTTGAAAGTCTGGTTAATAAC
GAGTTCTCTAGCGCTAGTGATGTGTGGGCCTTTGGAGTGACGCTGTGGGA
ACTCATGACTCTGGGCCAGACTCCCTACGTGGACATTGACCCCTTCGAGA
TGGCCGCATACCTGAAAGATGGTTACCGAATAGCCCAGCCAATCAACTGT
CCTGATGAATTATTTGCTGTGATGGCCTGTTGCTGGGCCTTAGATCCAGA
GGAGAGGCCCAAGTTTCAGCAGCTGGTACAGTGCCTAACAGAGTTTCATG
CAGCCCTGGGGGCCTACGTCTGACTCCTCTCCAATCCCACACCATCAGGA
AGAAGGTGCCTGTCGGGGCTCACTTGAAGCCTGTCAGGGATGCTTTGTAT
CTAACACAACGCCAACAGAAGCACATTTGTCTTCCAGAACACCGTGCCTT
AGAAATGCTTTAGAATCTGAACTTTTTAAGACAGACTTAATAATGTGGCA
TATTTTCTAGATATCACTTTTATTAGGTTGAACTGAAAGGGTTTTTGTAA
ATTTTTTGGCCAAAATTTTTTAAAACATACTTACTTTGGACTAGGGGTAC
ATTCTTACAAAATAAATAAACAGTTTTTAAAATTGTTTAGACACAGATAT
TTGGAATTAGCTATCTTAGTGCCAACTGCTTTTTATTTTTTTACTTCATC
AAGGTGATGTAAGTGACTCACCTTTAAAGTTTTTTTAGTGTTATTTTTTA
TCACTACTCTGGGAAATGGTTTGTCTTCAAGATGCAATACTTTTCTTAGT
AAAGGAAAAACAGCATAAAAAGATACCTGGTCTGCCTTGTACAAGAAAAG
GCAATATTAGAGGAAGAAAATTTAAAGAAAAGCTAGAGGAAAAAAAAATT
TTTTTAAAAATACTTATTAGAAGCAAACTGCCCTTGCATGGAAAACTGTT
TATTTTTTTCAGTGAAAAGGAATTCTGCTTTCGTGTTTTTGGGAAAGCAG
GAACTGAGTTCATTACATCTTTAATTTGGCAGAAATTAGCCTTTCTGTGA
ACCAGATGTGGTTTGGGGCAGATCTGTAGTAAACAATGGTGATTTTATTT
ATTTTTACTCTCTGGAAAAGGAGATAATACAATTCCAGAAAGTGAACTCA
TATTTCTAAGGTTAAGATTCCCTTTTATTGCACCTAGAATAGTGCTATGC
ACAGAGCGGGTGCTTGAGTTGTTGTCGTTTTTTGTTTGTTTTTTAAATGT
AAACTGGTAAATTTTGTGCTTATCTTCAAGGCTGGCTTAAGTATAAAATT
GTTTTTTAAACACTTGAAAAATTAAAGGATTTGTTTTATATT

Slco2a1, or solute carrier organic anion transporter family, member 2A1, encodes a prostaglandin transporter that is involved in mediating the uptake and clearance of prostaglandins in numerous tissues. The Slco2a1 cDNA sequence in Mus musculus is provided in GenBank Accession No. BC035200.1 (GI:23273774), incorporated herein by reference. The Slco2a1 amino acid sequence in Mus musculus is provided in GenBank Accession No. AAH35200.1 (GI:23273775), incorporated herein by reference. The Slco2a1 cDNA sequence in Homo sapiens is provided in GenBank Accession No. BC041140.1 (GI:26996626), incorporated herein by reference. The Slco2a1 amino acid sequence in Homo sapiens is provided in GenBank Accession No. AAH41140.2 (GI:37572262), incorporated herein by reference.

An exemplary DNA sequence of the Slco2a1 gene in Mus musculus strain C57BL/6J chromosome 9, GRCm38.p1 C57BL/6J is provided below.

NC_000075.6 (GI: 372099101: 103008489-103087849):
(SEQ ID NO: 7)
TCAGTCTCCACGTCTCGCTCCCCATAGCCCGGCTACCGCCGCTGAAGCCTGAAGCACCCAAGTCCCGCAG
AACCTGACCCCGGCCTGTCGCCACCACCTTGGCCCGGTCAGGGGACGTCGCCTCAGTCACGATGGGGCTC
CTGCCCAAGCCCGGAGCACGCCAGGGCAGCGGCACCTCCTCGGTCCCAGCCAGACGTTGCTCCCGCTCGG
TCTTCAACAACATTAAGGTGAGTAGTGTTGTGAGTAGTGTTCGTGTTCGGGGCAGGAGGGCGGCAGAGTG
GGTGACACAACCTAGGACGTCGATGAGGCCAGTCCGGGAGCTCTGGGTTGCACCCGGCTCCCTTCTCTGT
CCCCGGTGCCACATCCGTGGGGCCTGGCCCGGCGCGTGAGCGTGAGCGTGAGGGGGCGACACCTCTCTCT
CTCTCTCTCTCTCTCTCTCTCTCTCTCTCTCTCTCTCTCTCTCTCTGTGTGTGTGTGTGTGTGTGTGTGT
GTGTGTGTGTTGCGCGCATATGCTCATAGGCTCTCGTGGGGAGTAATAATCAGAACAGACGTAACTTTGT
AAAGGGGAACTGTCCTGTTCTTCCATGGAGAAAGAACACCTCCAGGGAATTCTGTTGGGTCTCTGCTGTG
GGTATGCGCTCTTCCTTAATGAGAAGGGATGCAGAGGAGTTTAGCCAGCACCTTGTGGGCAACAGGCTGT
GGGCAGCCTGAGATGCAAGTCAGAGCACAGGCACCTTGTCTCAGTGGATGGTAGAAGCCTGCATTCCTTG
CCTGGGTTTTCATCTTTGCAATAGTTGGGAGTCGCAGATTAGATTGCTACCTGGCTGCATAGCTCCCAGC
AGTTTTGAGGATTCCCCAGCTCTCCTGGTCTCAAGAAAACAGCAGGCACTCGAGGTTTATGGAGAAAGGC
CACCAGAGGTGCCTGTGGCCTGAGAGTTATGAGGAGTTAATAGAAAAAAGAGGAAGCTGTTAGCATGAGA
ATGCCTTCTTTGGTAAGGGTCAAAAGTAGGGTCCACTTCCCCCAGGTCTTGAAGGACCTGGAATGGTTGC
GTCCTCACAATATTTGGGAGAGAGGTTCCTGAAGAAGGTATCTCCTGGAAGGGGCTAGACGACCTTGGTC
AGACATCCAGGCGCCCTGGGGTCCGGCAAGCATATGGTGCAGAGAGATTATTTCCCACAATTCCTACCTT
AGATGTAATGGGTGCCCAGACCTCACATGGAAGGATTGGAGAAATGTGAACAGGCTATGTGTGTCTAACG
GAAAGAGACTGGGGTTTGGGGGGTGGGGTGGGGCAGAGCGAATGCCACCTGCATGTTCTGCCTCAGGCTG
TTACTGGTAGAACTGGGGTTCCTCGTGGGGATCCCTCTTTTGTTACTACCACCACCACCACCATGGTTTT
AAAGGGCTACCTTGATTTCCCATTGGAATATTCCTAACACAGAAAATTGGAGGTAATTGAAAACAGTCCT
CAGATCTCCTCCCCTCTAGCCAGACCTGGATGGGAATTCCATTTATTATTTTGGTTCTACCAATTTAGAG
GGACTTGGAGAATTATGGAGATTTTTTTTAAAACTTGCTAATTAGAAGGAAAGGGTGTGTCTAAAGGATG
GGTGGTCTCAAAGGAATGGGATTGTGGCAGCACGAGAAGGAGGTCACTCTCATTTTCCCCAAGAATGGAA
AGAGAGGTGGCTGTCCCAGGATTCCTGATGGTGAGGGTGACAAGAGTGACTCAGCACCTCCAACCCCTAA
GAATAAGTGGGTGGCAGCCATTTCGTTGAAAAATATAAAAATAAGATAAAACCCACATTCATATGCTGAG
ATTCATGCCTGAGACCTGGACCTAGGCCAGTTGGCCTCAGAAGGGAGAGTCAAGAGCTCTTATCAGTCAA
GAGGTTTTGAGTCTGTTAAAACATTTCCTTGGTTGGAGGATAATAAGCATCAGATGACAGGCAATTTGGA
GAGCCCCCACCTTAGCCAGGTTCGTCCTGGAGACCCTAGGCACACCTGAGTGGAGATCCTAGCTAAGCTT
TTTCTTGCTGGTGACTCTATGCCTGGGCCTTTGCTATTCGAAGGGAGATTTTCTCCTCTTGATAACAGGC
TGGGTGATTACCTTATACCTTCCCAGCAAGGGAAAGGTGACACAAGTGACACAAGTGGGGCAGGTGACAA
GGGACATCCAGCCCAATAGCTTGTCACTTTGCTGGCATCTTTGTCACTGGGATGGACTGTCAAGAGCGGA
TGTTGCTATAAACCCATGTGCCACATGATCTGAGGGAGGAGTAGAGTCTGAGAATACAGATCCACGGCCC
AGCCCGGCCACACTCACTTCCTGGGTCACTCAGGACAGACACATGGCTCACCTTTCTTGGCCCTCTGGGG
TCCTTTTCTGAAAGAGTGGGTGTCATGAATAGTTCCCACCATGCACCTCAGTAAGAATGCCCTAGGGTAA
GAGTTAAACTGGGCCTCCCCAGGGTGAGCCCCACTTCTGTAAACAAGGCATCGCATTCGATGTTGTCCAG
GAGCTCCGGTTTCAATGTTAGACCAGCTCTGAATGACGCCCTGGTCTCCATCCTTGACACCACACTGTGA
GCCTGCACAGGTCAAGCCAGATAGGAGGGTACTGAGGTGGGAGAGTGAGTCCCACCTCTAACCAAGATGC
TATTTGTAATTGATACTTACCAACAAAGGCAAACATCCGTTTTCTTCAGTGGAGTGTCCCTGTGAATATC
AAACCACACTTCAGAGCAGACCCGTATCCCCGGGGTAGCTGGCCAAGATAAAACAAAACCCATTTTTGTT
TGTTTGTTTGTTTGTTTGTTTGTTGTGGGCTTTTTGTTTAGCTTGGGCATTTTTAGCTTATTGTTTTGTT
TTGTTTTGATTTTTGTGAGATTTATTTATTTATTTATTTATTTATTTATTTATTTTAAAGCCTACAGCAC
CCTCTGGTCTCCCAGTAGTAACCAAGCCTTACCCTACTTAGCTTCCAAGATCAGACGAGATTGGGTGCAT
TCAGGGTGGTATGGTCATAGATGAGTGCTGTTTTGTTTTGTTTTGTTTGAAAGAGAGAGAGAAAGCATAA
AGTTGAGTGGTTGGTGAGGTGGAGAGGACCTGGGAGGAGTAGGGGGAGAAAAAACATGATCAAACTATAA
TATTGGACAAAAGTTGTTAAAAATCTTTAAAAATGATTAAAAGATAGATTTTAAAAACTCAAAATAAAAA
TTAGAAAAAAACTGGGTGTGGTGGTCTTGTAATCTTAGCATCCAGGAGATAAAGGCAGGAGAACTAGAAG
CTTAAGTTATCACTGGCTATAGAGTAAGCTGGAGACAGCCTGGGCTACATGAGACCTGTGAAAGAAAGAA
AGAAAAAGAGAGAGAGAGAGAGAGAGAGAGAGAGAGAGAGAGAGAGAAAGGAAGAAAGGAAGGAAGGAAG
GAAGAGAGAAAAAAGAAACCATTGTCCCATTAGTTCCATGATGCAGTTATTAGTTATCAATGACAGTTAA
TAATATTAATCACAATGTCCATAATAATAACAAATAATAGCTAACAGTAATTGAGCTCTATGAATGTACA
GACCTTGAGGATCTTGCACGCACATCCCATCTGCTTTACAACTCTAGCAGAGGATAACATTACCAACCTC
ACTGGTAAGGCAATCAATTCTTCATGGACCAGGAAGGGAGGCAGGGAGAAGCAGGAACAGCTATGGCACA
AAGTACTGTCCTCATGAGCACTATCGTCTACGCTGGCTCTATAACTGGCGACAACACAGCCATCACTTAA
CAGTACTTTCTCCAGGACCAGAAGACATTCAGAAGTGGGTACAGAGGGGACCCAGACATGAATCCCCAAT
GCTGACCCACTCCTGCCTCTTCTCTCTTCACAAGGCTCCATGAAAGAGCCTTGCACCTATTTATTATTAT
TATTATTATTATCATCATTATTATTATTATTACATGAAACGGTGCTGGAATTAAAGGTGTGAGGCACCAC
CATGCAGCCAGGGGAATTTTTACATAGCCTGTGTGTGTGTGTGTGTGTGTGTGTGTGTGTGTGTGTGTGT
GTGTGTGTGTGTGTAACAAGTTGATGTGCAGCTATGTGGAGGCCAGAGGACAATCTACAGTATCAGCCTT
AGGGACAGCTTCCATCTCCTTTGAGAAAGGGTTTTAAATGGCCTGAAGCTCACTGACTATGGTCAACTAA
GCCATGGATCCCTAGCCATGGATCCCTAGGGCTCCCCTGTCTCTACCTACTGCTAGGGTCAAACCCACTC
TGTGTCTGGGTAATGTCATTCTTACACAGGTACTAAGGCTAGAACCGGGATCCTCCTGCGCATGCGGCAA
GCACTTTATAGGTTGGGAAGTGTTTTGCTTCTGGTTTATTTTGTGGTGCTGTTGAATCCCAAGCCATGAG
CCATATAAGAGAGCATTCTACCACTGAGTCACCTCCCAGGCCCTGTGGGGTGAAGATTTATATATCTCCT
CCTGCAGGAGACCTCTCTCTCTCAAGATTTATTTATTTTGTCTATATGAGTACACCATTGCTCTCTTCAG
ACACACCAGAAGAGGGCATTGGATCCCATTACAGATGGTTGTGAGCCACCATGTGGTTGCTGAAAATTGA
ACTCAGGACCTCTGGAAGAGCAGTCAGTGCTCTTAACCATGGAGCCATCTCTCCAGCCCCCAGGAGACAT
CTCTTGAGGTTTGGTTTGCAAGAAGATTCTTAGGAATCTCAGAACCTGCTCAAACATCACTGTCAATTAG
TCTAGTACAGGCCCAGGACTCTAAATTAAAAGCAGGTTCCCTCGGAGACCCTGGGAGGAGGTAGGCTCTG
CTCCTGACTTCTCCTGGCAGCCTCAGACTAATCTGAAGCTCCCTAAAGTGAAACACCATCGTGGAGCTAA
GTCGAGGTTCTTCTGAGGCCTTTGACCCCCTCAGCCCTAGTGAGGCATCTTGAGTGCCACTTCTTAGTTT
CCAAAATCCAGGGCAGCATGCCAACAATCTAATCCATCCCCAGGAAAACACTCCCACTGGACTCCCTGAA
TCAGAGAAGAGCTAACGGTGGCTCGCCCACCTCCCTCCAGGGGGCGCATTTCCATAGACTTCAGAACAGT
GATCTGTTCTCAGGAGGCTGGCTTGGCAAGTTTATCAGAAGGCAGGGAAGGAAGGATGAGGACTAATGAG
TTCTCTGGTATTAGAATACTGGGATTCCTCTCTGGGATGATGTTCTAAGCCCTCTATAGCTTTTCATCTC
CCAACACAGAACAAATGAGAGCAAGTTAACATTTCTGAGGGTGCAGAGTCCCTTTCATCTGTCCTGATTG
TGACAGGACTTTCTCTTTTTCCAAGCTAGGAGACCCAGCCTGGGCTGTTGGGATGAGGGCACGCTTCTCC
TATCTTCTCTTGTCTCAGCATGTAATTTGAGACAAACGGTGTTTGTAGGGGAAAGTTAGAGGTCTCTGAC
CCTCCTCGTTCTCCCCTTCCCACCCTTGAATTGGTTGTAACCACCTTTTTGTCTGTAGAGACACTGGGTG
CACAGTGTCTACTATGTGCCCAATCCCAAGGATGGGGAGGGAGGTACCAAGAAGGCAAGGATAGGGAGAC
TCAGCGCTGTGTAGATAGAGGATAGGGGATAGAGGATTGCTCTCTGTTTATTATTTTTAGACTCTCTAGA
GATGGTTTTCTCAAGGAATGCTGTGAAGAAATGAAGAAATGGGGTGTATGAGTTTGCCCTTCTTCATGCT
CTACATGGGTCACCATCACTGGGGGCTCTCTCACAGTGGCCCCATGAGAAAGTCACTTGTGTTGCTTAGT
TCCAGGCACAGCTTTATTTGTTTAGAGTCTTTTAGAAAATGGTTTTCAGTTTGAAACGATAACAGATTCT
CAACAAAGCTGGTCTTAGAGGTCCTGTGTATCCTACACCCACTCACCCTCAGGGTTCCATGCTATATGAC
AACAGTAATGACGAAGGATATCAAAAAAGCAGGAAATCACCACTGCTATCTCGTGCACGTACAGTCCGGT
CCTGTACTGCTTTATCACAGGTAGATTTGTGCAGACACCACCCATCTGAGGATACAGGTGTGTTTCACCA
CCCACCGCAGAGAAGTGCCTTGTGCCTGCTCGTTGAGAGTCTCATCCTCTGACCACTGTCCCTAACCCCC
AGCACTGTCTAGTGTCCATCTCCAGGATTGTATTATTTCAAGGCTGTTACATACAGGGAAGCATGTAACA
GGCGACCTTTTGAAAAACCAGCCGTCTTCATGCAGAATAACGTATTTGCGTTCCAGTCCGTCTAAGCTGT
GACACAGATCAGGTGCTGGTTCCTTTAACGGCTGAGTACTACGCCAAGGTGTGGATGTGCTGTGCTTCGT
TTACACATGTTCACTTATGGAAGAACGTTTGGCTCATTTCCCACTTGGGACTAGTTCAGATAAATCCACA
AGAGTGGTGACGTACAGGCTTCTGTGTGGACATAGCTCGTAATTTCTCTAGGGTGAATGCCGTGGAGTGT
TGCTGGGTGGTGTGGTTGTTGAACAATGATACTTTTTTTTTAAAAAAAGATTTATTTATTATTATAAATA
AGTACACTGTAGCTGACTTCAGACACACCAGAAGAGGGTGTCAGGTCTCATTATGGGTGGTTGCAGAGAT
TTGAACTCAGGACCTTCCTTCGGAAGAGCAGTCAGTGCTCTTACCCACTGAGCCACCTCACCAGCCCTGA
ACAATGATACTTTTTTAAAAAGGATCTTCCTAACTCTTTCCTGGAAGGGTCGTCTCACCTTACACGGGTG
TCAGCTGCTTAGCAGTTGGTCAGCTTTTGACATTACCACTCTTTTTTTGTAAAAATTCTAATAGACATGC
GGCACCACCTCTTTGTGTCTCGTTCATGTTGTCCTTGTGGCTGGAGAGAGGAAGATTGAGCTTCTTTTCA
TGTGATCATCGCCCCTTCATGTTGTCGTTTTCCTTTAATGTTGTGTAGCTTTAAGAGCTCACTGGGCACT
CCAGTTATAAGTCCTTTGTTGGGTTATTTGGTCTGCAGATTTTGTCTTTGAATCTGTAGGTTGGTTTTTT
ATTTTTTTTAATCATTTAAAATGGTTTTATAGAGAAAATACTTAATTTTGATGAATTCCAATTATTGGTC
TGTGTGTGTGTGTGTGTGTGTGTGTGTGTGTGTGTGTGTGTGTGTGTGTATACACCTGCTCACATGTGAG
CACACAGTAGTCAAAGGACCTTAGGTGTTGGTCCTCAACATTCACATTGTTTGAGACAGGGTTTCTTGTT
TACTGCTATATACAACTCCAGCCTTTGTTTGTTTGTTTGTTTGTTTTTAAGCTCACGGAGGCAGATTACC
CACTACTCACTGTGTAGCTCAGGTTGTCCTGGAATTCCTGATCCTCTTGATTTCTATCTCTGAATCGCTG
GGGTACTAAGTATGTACTGCCATGTCTGGCTTACATATATCAGCCTTCAGCAGTTTGGGTCCTCCTCTTG
GTGCTACATCTAAGAAGGCTTGCTTCACTGAATCATAAATCTATTGCCTTTCGAGTTGTTCTGTGTAGAG
GTGAGGACCGGGTCAAAGCCTTGTCTGTTTTTACCTCTAGATGTTCTTGTGCCACGTCTCTTCTCTCCTA
ACCTGTCTCCGCGCTCTTGTCCAAAGCCAGTTGGCTGTTCTCCTGTAGGTCTCTTGGTTCTTCGTTTAGC
TTCACTGCTACACACCTTCAGGCCCCTCCTTGTATCGCTGTCTGATTATTGTAGCTACGGAGCGAGGCTC
GACGAGGGAGGAGCTTGCTTTCACCTGTAACGGTGCAGTTATCAAGCTTGCTAACTCACGGGGCTTCCCT
TGCCCTCTGGCTTTTGGCATGATTTCAGGGCAGATTTCAGCAGTGGGGATGAGCTCTCTGTCAGCACCCC
TGTCTCCTGCTTGCCTTCTGGTTTCAGCTGTTTGGCATGCTTTTCTCCTTGGACTTTTAGATCTGTGCCT
CTAAATCCTTCTCTATGAGGGTGGGAGATGTCTCAGTCTGTAAGGTATTCACCATGCAAGCATGAGAACT
TGAGTTCAGAACCCCAGGATACATGTAAAAATCTGGGAGCCATGGCCCAGGCCTCTAATTTCAGTGCTGG
GAGGGTAGAGATAGGTTTCCTGAGGCTCCCTGGCCAGCCAGCCCAGCAGAACCCATAAGTTCCAGGTTCA
GTAAGAGAATTTGCTTCAAAACTAGAGACTTGCTGTCAACCTCTGGCCTCCACAAGCACATGCATGTGCA
CTTGCACATACATGTATACACACATGTATACACATGCAATAAATACACTTTTCCTTTATAATCCTAATGA
CAGCCCTGAAGAGAGGTGTCAACCCAACACCTCCATCTCACAGCTGAGGAAGAAGAGAACTGCACAGGGC
CGAGTGCTGAGTAACTGACAAAGCAGGAATGCCCACTCAGCATCTCCCTCCCAAGTCCCCCTTTTCCCGT
GACCACAGAAGGACTCTGTCTTCTACTTTCTCTCATCCGTGACCTGCAGGCTTGGGCTCCATGGTCTCAG
GGTTCTTTTCCCAATCTCAAATTCTCAGCTCATGCTAGCCATCATGGATTAAGATGAGACTAGGAGCGAG
CCAGTTGGATCTGTACTGTGCTTGGAGAGTTTCTGTGGACCCAGTGGAAGCAATGAGATTCTTCCATCAG
TGTCCTGCTGGCTTGGGTTGGAGGACCTTGGTGGCCTGGGTCTGCCTTACTGTTGGAAGTTGATTAAATC
TTCTCAGGGGTGGAGAGTGAAACTAAGGCAGAAGTCAACTTGGACTTCACTGTGGGGGTGAGGGGGGTGG
GCTGATACTGTTGGGGGTGGGCATCTTCATGAGCTGATCTTCCCAGCTCCTTTCCATTCTGGGTCCTGAA
GCTCAGCAGTATACCCAAGAGGCTCAGGTCCAGCCCAAGGGTGAAGCAAAGAGAGGGAGCCAAAGGACTT
GTGAGGAACTCTTAGAACATTCAATGCGATCAGTATTTGGTGCTAATGCCCAGGACTGCTCTTGGCTTCA
AGAGATGTGGTGGACTCTCTTCTCCTATCTTAGGGTTTCTATTGTGGGAATGAAACACCATGACCAAAAA
GCAAATTGTGGGAGAAAAGGGTTTATTTGGCTTAGACTTCCATATTCCTGTTCATCACCAAAGGAAGTCA
AGACAGGAACTCAAACAAGGCAGGAACCTAGAGGCAGGAGCTGATGCAGAGGTCATGGAGGGATGCTACT
TACTGCCTTGACTCCCATGACTTACTCAGCCTGCTTTCTTATAGATCCCAGGATCACCAGCACAGGGATG
GAACCATCCATACTGGGCTGGGCCCTTCTCTATTGATCACTAATTAAGAAAATTCCCTAGAGCTGGATCT
TATGGAGGCATCTTTTCAGTTAAGGTTCCCTCCTTTCAGATGACACTAGCTTGTGTGTCAAGTTGACATA
AGTCTACTTTAGCACACCTCCTTAGAGAATTAATGAGCATCTGGGTCCTGGAGGAGCCTGGGGTCTTCTT
GACAAACCACATATGTTCAGCAGGGTGTCACACCAAATGTCCCTGGGATATGGGCTAGCCTAGAGCACCA
AGAAACTTGGGTAAGATACAGAGGTCCTCAGAAGGGGTCAACATGGAGGTTCACCTTTGTCACATGCCTC
ACCTCAAGGCCCTGGGTTCTAAATAATAAATAGGTTTCCACATCCTGGAAGGGCTAGAGAACAATGTATC
TGGAGATAGGGCCTGACCTGACTAACCCCAAGAATTAGATTTTTCCCCCTGAAAACCAAATGAATATTAT
ATTTTGTTTCACAAATCATCTATTTCTACAAGTCTGTTCTTGTTCCCCAAATCTTGGAGTAACCTCAACA
TCCAGGGACCTGTTTTGTGTTTCTCAAACGCAGGGTCCCAGAAGAAGCCCGTCAAATTTGGGGACTACCA
CCTAGATAGAGCTCAGGAAATAACTACTTTATCTGGCTAGTTCACTCAGTCAGCTTGTTGGCTACAGAGG
GACAGACTCTCCACAGTTTAAACAGGATGGGAATGTGGGGTGTGGGTCAGGACATTTGAGCCCAGTGGGA
CACTGGCAAAGTGGGACCAGTTACAGAATCTGGTGGGGTGCCCCGGGTTGGGGGAAAAGGGCACGTTGGT
AGATCGCTACCAACATCTACCAGTAGAAACGACACAGGATTTTCCTGGTGTCTTTTGGTTGCCAAACAGA
ACCCTAAACAGATCTTAACTGACCACTTTCTCAGTCAGCTTCAAACTGTTCCTTCCTTGCGCCTCGGGTC
CTCTTCTTTTTCTTAAACTTACTGTTTTTCTTTGGCTCACAATGCAGGTGGAGGCAATAGCCAGGAGCTG
AGCCAGGAGTCTGCAGTTGATTGACTGGTTGTGGGTGAGACCTTTTCTAGCTAGGCACTGCTGTTCTGTC
TGGCTCTCCGAGAAGCTCCAAAGTTTCTGAGATGGATTGCCTGTATGTTTGCTGGTTCCCAGCCACCCGA
ACAAACCACCCCCGTTTGCCCTGGGACACAGGACTTTCAGTTTTAAAACTGGAAATGTTCTAAGGAAATT
GAAATAAGTGAGTCAACTTACTGCCTCCCATGTAAGAATAGCTTACGAAAACAAGCTTCTACTCTCTGAC
TACCAGAGGCAGCAAGGAGGGTCGCAGATGATGTTGCAGGGTAGAAATGAACCCCAGAGGCTCATGGAGG
CTTCCAGAGAGCAGCTTATCATGGGCCAGATCTTGTGCCTTGCAAGCCTCATTAGTCTCTCCGATTTACA
GGTGAAAGCCTTTCTCCAAGCTTCCTACGCCCATTGTGGATCATGGTTCATGACTAGTCCATTGCGTAGA
ATCTGTACAGATAAGAATATGGACAGATAAAGCTCCTGGTCTGGGGAGCTATTGAATTTCAGAATTGACT
GTGATGTGCAAACTCATCTCCTCAAAGATGCAAAAATGTGCTGATGTCTCTGTCTTTGCTAGCTGGCTCA
TTCACAGGATAGCCTTCTTCTACAACCCAGGCCCACCTGCCTTGGGGATGGCACCGCTCATGGTGGATGA
AGTGCTCCACATCCACCGTCATTAAGACCATCCCTCACAGGCATGACCAAGTAATCCCTCAGTGGGAACT
CTCCTCCAAATGACTCTAGGCTTATCAAGTTGATAGCTGAATCTAACTAGGATATAACCTATTATATACT
TAATGCATAGGTATATAAGATTTATAAGGAGCAGGGATAGCATTGTTCATACTTGTTTGGTTGGTTGTCT
TATTTGTACTATATTTTAGTCTAATTTCGTTTCATTCTTGCATTTTTTCTTTCTTTTCTTTCTTTCCTCC
TCTTCCTCCTCCTCCTCCTTCTTTTTGAGACAGTATCTTGCTCTGTGGCCCAGACTGAGCTGAAATTCAC
TAAGTTAACCTGGTGTACTTTGAACTTGCAATAATCCTCCTGCCTCTGCCTCCCAAGTGTGATTACATAC
ATGAGCCACTGTGCCTGGTCTTGAACATTCAGAGTACTGGCCACAGTTCCAACCCATTCTTAGTAGCCAG
TACAAAATTCCTTCCATTTGGACCATGTGCGTGGTGATCCATAAAAAGAAAAAAAAGGACAGATATTCAG
CTATGACTATAATTATCAATATATCTGTGCTTTCTGTAAGAGTGCACACCCACAGTGCACACATTTAGTG
AGCTAATGTGAACAAAGCTAGGTCTTCCAGAACGCTGACTGATGAAGCCCTTTCCCTATGCCGAGAAGGC
AGCCTGTTAATTCCTACTTTATCATGGAGGAAACTAGGAGGAGGCCTATATGAACTGCCACTCACACAAG
TGGAGAGCTGAAAGTATCGCTTTCTGGGGGTGCTGTCTTGAGTTTTTCTCCCCTCTACAGCTGCACATTG
TCCTCCATAAATCTCCAGTCATTTAGCCAGAGTGGTACCTAGCCATAGCGTCCTACCAGAGATGTGGCAG
AGCTAGTGCTGTTTTCTAAGTCAGACATATCGACTGTTGTCCCAGAACTCTAACTTCCTCACGTTTGCCT
CTAGATGCACATTTCCTCTTTGGTGTTCAGCCAACACCAACACTCCATCCAGCACAGGACACTTAGATGG
AGTGTGGACGTGGGGCTATGTCATGGCTTTAAAAATGTTGATTTCTAAATGAAAGAATTAGTGGAAACCC
GATCATTTTCACAGTGTCTTAGGCTGTAACTCATTTGCTTAGAAACTCAAAGGGAAGGACTACTCTATCA
TCGGAGTGTCAATAAATAAACTGCAAGATGTCTGTGGAATAGTCGCAAATCTATTGTGTGTACATGTCTT
GTTGGGAATTGTACTACACATCAATTTTAAAGTTTTTTTTTAACTTCTTATTGATTCTTGTGAACTTCAC
ACCATTCACCCTAGTCCCACTCATCTCCCCATCCCTTCGTACCTGCCCTCTTCCCTTGCAACCTCCCCTG
CTACAGAAGCTGTAGTGTGTCCCAGTGTGTCCCAGAGTACACTCTTTCGTCCATACTTATTTATTTGCCA
AAGTTCATTGCAATGACTTTTTGGTTTGGTACAAGGCCTCTGACTCCTGCTACTGTATCAATACTGGAAC
CTCTCTGGAACTCCTCTTAGATATCCTGTTGTTGCTCTGTGTCATGGTGATCCTGTAGTTTTAGATCTGT
AGGACTGACCCAGCCATTCATGCACTCCAGCAGTTCATCAATGGGGTAGATGTTGGGGTGGGACAATTCA
AAGCCCTGGATCTGGGCCTGAGAGGTACCAGAGCTGGTCAGCCTTCTGGCTCTCCTCCTCTCATTGCCCT
TGGAGACAGCTCACCAGCGTCCCCCATATCCAGACCAGCTCGGCTGTGCTGCCCAAGGAGGTGCAGGGCC
TGCTCTCCCAAGTGCTGCAGCAGGGGAGGGGCAGGGCCAGATCTCCTGCTCTGATGACCCAGGGCAGCTC
TCCTGCTGCCAAAGGTAACAAGGGGTGAGGGAGGGTCATCTCTCCCTAACCAATGTCACTACACAACAGG
CAAGGGACGGGGCCAGCTCTCCCATACATGCGCCCTTGGAGCTGGCTCACCCACAACCCTGCTATCAGGG
GCAACTCTACTTCGCTGCCCAGGTGAGGTGGAGGGCCCAAGTGCTGCCGCAACATGACCTCCTTTCATGA
CCCCAGGGGCAGCAATCCCATGAAGCCCAGGTGAGGATAGCGTCAGTTCTACACAACACTCAGACATCAA
CACGTCCCTGGGTGGTATCCCAGACCAGGGACATCTGCCTAGCTGCTGCAGGGTTACAGACCAAGATGTG
CCCTCTCCCTGTGACAGCATAGGCCAGGGCCTTGCTATGGTCCCAGGTGGCACCACCAGCTGCTCACATC
AGGTTCCTCATGACCCTTGAGTCTGCAGTTCTGCCTTTCTTCATTGTGCCCACATCCTTCTGTTTCTTGT
TCTCTTCCTTTTCTCCACCACTGACTTGCTCCTCTTTGTGGTGTGTGTGCCTGGAGACTCTGAGTATCCG
GGGTCACCTCAGGAGTGCTGTGCCCTGATTGTGCATTATGGTGCCAGGCAGGGGTTATCTCGGGCACAGT
CTGGCCACTGCTGGACTAGTGGACATCTCGGGCAAGATCCCTGTCCAGGCTTCATGGTGCCAGTCTGGTG
GTCATCTTGAGCTCACTCCTCTCCCAGCCTGCCAGAGTGGCCCTTTGCAAGGGTCATCTGTCTCAGGCTC
ACTGCCACCTGGGACCCTCAGTCCCAGGTGGGATTCTTCTAGTCTCCAGCTCACTCTCCATCCTGGGAGC
CCATGTGGTCCTGCCTGGCAGCGGACTGGTGGTCATCTCAGGCTCAATTATTTCAGGAAGTGTTAGGCTA
CTAATCATTCAGATGTTCATAGGTCAGAACACCAAGCACAGATATAATCTCTTCTTTTTGCCACCTACTG
ATATGCCTGTGACAGAGCAGCCATGCCTGTCACACCTCTAGGGACAGGGAAGCTTTCTAATGATTTATTT
ATTTATTTGTATTTTCTGTGCACTGGTGTCTTGCCTGCATGCTTGTCTGTGTGAGGGAGACAGATCCCCT
GGACTAGCATGACAGACAGTTGGGAGCCGCCAAATGGGTGCTGGGAATTGAACCCATGTCCTTTGGAAGA
ACAGCCAAGTGTTTTCAACCACTGAGCCATCTCTCCAGGCCCCAATCTTAATGTTTTAAGAATAAATCAA
ACAATGGAAAGGAGCTTCTCTGATGTTAAGTAAAATGAGGCAGGATGGACCACCGTATGGACCAAGGGTG
TGGATATTTCTGCTAGGGCACACAAAGCAAGGCAGCGCTAAGGAAGAACACCCAGACAGAAGTGGTTCTC
CGCTGGGCGGGGACAGGAAAGCCTAGCAACAATGTCCCTTTGTTCTTTATGGATTTGTATGTTCTTTATT
GTCCTACAACAAACATGTCCTATATTTATGCTTAGATGAAACTATCAACTCCAGACTCTAAAGTGACATC
CACCCTCCACTGCCAGGTGCACCAAGACATTACACTGGTGGTACATGAAATAAAAGCCATGGAGCACAGA
AGTGCTGTTTTCCAGGACTCTGAGAGTGGACCAAGGACGCTGCAGGATTGCAGTGAGAGGGATGGGGTTG
TCCTGTAGTGTGGTTGCTATTCCCTTTGCATTTTTACGCCTATTATTGCATTCGAGGCTCACTGCTTCCC
AGAGGTGGACTCAATTCAGATAATGATACTTGAAGCCTTAAAGAGTGGTAAGAATGCAGAACACTCTCCA
TTCTGGAAAGATGGGAGACAGGTATATCCAGGAATCCCTCTCCACTCTGCCAGCACCCACTCACAGTGCC
CAGCTCAGAGCTTAGGAATCCAGCCCCAAGGGTTGGCCAAACACCTTAGATAGACGAGTCTATCTATCAG
ATCTGCATCTTGCCCTGTAGGAAGGGCAGCCAAGGGCGAGGCAGCAAGGACAAGAGCTGCCACTGGATCT
GCTGAGTACTGACTGGAGGTCTAGTCCTGTGCCTGGGGATTTATGCCTGGGAAGACATGGAAACCATCTT
AGTGGGCTTTCCTGTGGCCATGTTAGTAAACCTGGATTGCCTGCCACATTGTTTGTAAGGCTGCACTCTG
CAGTTGTTCTAAACAGCAGTGACCTTTGACTCTCTGAGGCCAAGAGTAGTAAGGTTCATTTATTTGTGTG
TGTGTGTGTGTGTGTGTGTGTGGTCAGGAGTCACCTGGGGTTTTGTTGTTGTTTTGTTTAAGACAAGGTC
TCTCACTGGCTTTGAATTCATTAAGAAGACTACCCTGGATGGCCAGTGTCTTAGGGTTTTACTGCTATGA
ACAGACACCACAACCAAGGCAAGTCTTATAAAGGACAACATTTAATTGGGCTTGCAGGTTCAGAGAGGTT
CAGTCCATTATCATCAAAGCAGGAACATGACAGCATCGAGGGCAGGCATGGTGCAGGAGGAGCTGAGAAT
TCTACATCTTCATCTGAAGGCTGCTAGCAGAATACTGACTTCCAGGCAGCTAGGATGAGGGTCTTAAGCC
CACACTCACAGTGACACACCTACACCCAACAGGGCCACACTAACTACTAGTGTTACTTCCTGGACTGAGC
ATATACAAACCATCACAGCCAGAAAGCCCCAAAGGTCTGCTTTCTCAGTGATGGGATTACAAATGCACAC
CAACACGCCCAGCTGCTTCTTCCTTTTGTTCTGAGGATAAGCCCAAGGTCCTCATTACTGACTGAGCTAA
CTCCCCATCTTCACATTCTTGGTCGATTACTATCAAGCATTGGCTATTCAGTGATGAGTAAAAGCAGAAG
AGCTCTGTCCCTCCAGTCAGATAAACAGTGCACAGTTATGCAAATGACAGCAAAGTCACCAGGCATGTCA
TATAATCGGGGGAGGAAATTGCCTGTTTAAGGTAACAAATATGGTGGATTCTGCTTTACATATCTGGGTC
ATGGACTGTGAGTTTTAGCGTCATCCTCACTGTCCTCATACAGAATGACTGAGTGGGAAACACCATTAGG
AGCAGAAAAAATGTCTCTGCCTCCCAAAGGCTGTGGTTAGATGGCTGGATCATAATAAGTTCTAGTGCAT
TATGGTTTCCATCAACAAGACAGGCCCATGTAGCACAAAAGACCTCTTAATGAAAACACTTAAAGGTGCT
TCATAAAATAAGTAAAATCAGAGTTCCCATGTATAGCTGAGCTTTCGAGAAAGAGAGGGGAGTGGTCAGG
TGAGAGAAATGAAGAAGGCACAAAACAGCAGAAACTTGACACACCAGTCACCGCTGTGGGGGACAGATGG
GAATGGAGCATAGTGGCTATGAGAGCAGGAAAGAGCCCAAGAGACTGTCTGAGAAGGATGTGGTGTTAGT
TACAGTATGGGTACCTAAGATGACATGAGAGGCGGTGGAATCCCATGTATAAGCTACACGGAATTGCCAA
CTGAGGTACCACATGATGGACAGACTTTATACTATATAAAAGTTAACTCAAATGGATCTGAGACTTAAAT
ATAAGATCCAAAACTATAAAACTCAAAAGAAGACATGGAGATCAAGCTTTATGACACAGAGCTGGGCAAA
GCTGTCTTAGATGTGATGCCAAAAGTATAGGCAATATATATTAAAATTGTTAAAAGTTATATGATGTTGA
ATTTTTAAACATTTCTGAGTGTCAAGCAAGACAGTCAATGGCATCTTAGAAGAGGCAATCTATGGACTGG
AGAGATGGCTCAGCGGTTAAGAGCACTGGTTGTTCTTCCAGAGGACCTGGGTTCAATTCCTAGCACCTAC
ATGGCAGCTCACAACTGTCTATTCCCCAGCTCCAGGGGACACTACACCCTTACATAGGCATACGTGCAAG
CAAAACATAAAATAAAAATTAAAAACAGAAAATGCAATCTAAGACATGGGAGAAAATACATGCAAATTTA
TGTGCAGTAAGAATTAATATCTAGGAGCCAGGCATGGTGGCGCACACCTTTAGTCCCAGCACTCAGGAGG
CAGAGGCAGGCGGATTTCTGAGTTTGAGGCCAGCCTGGTCTACAAAGTGAGTTCCAGGACAGCCAGGGCT
ATACAGAGAAACTCTGTCTCAAAAAACAAAACACAAACACAAACAAACAAACAAACAAAAAAGAATTAAT
ATCTAGGACATATAAAAAATTTCTTCAATCCAGCAACAAAGAACAACTGGAACTGTAAACTGAATAAATC
CTATTCTTTACTTTGCTTTTGGTTCAGGTACTTTTATCCCAGCAATGGAAAACACACTAGGACATTACAG
GAGGAAGATCTGAAAGCAGAGGCTCTAAGCAATAGTTAAACACCCTTGATCATAGCAGCGCTAAGGTAGA
AGGAACCCAAGTCTCCACCAATGAATAAACAGACAAGCAAACCAGACATAGCCACAGGGTGGAACATTAG
TCAGCCTCAAAAGGAAAGACATTCTCACGCATGCTATAAAATGGACAGACCTGGAGGACGTTCTGCTCAG
AGAAGTATGCCAACCACAAAAGAGCAAAGATATGATCTCATTTAAGTGAGGTCTGTAACACGGCTAAATG
TATAGATAGGGACCGTGAATGGCAGTTTCCCAGGGTTGGAGGGATGGTGGGAGAATGGGGAACTCCTACC
CAATGGGTATTCGTTTCAGCATCACAAGATGAAGTGTCTGAGAGGCAGATGATGGTCATGGTTGCACAGC
CTTAAATGTGTGATGCCACAGGAGTTGGTCCTGGAAATGGTCGTGATGGTTGGTAGATTGAAATGTGGTT
TTCCTCCATTGGAAATATGTATGGGTGATCAGTAAAACATCACTAGACACAGGCCTCCCAGGGAGGGGAG
AATAAAAGGTAGACTTACAAAATGATAATAAAACAAAATGTAATCAAAGGCTGTAGCAACGGAAGGTTGC
GGAGGCAAACCCCCTTCGGTGGAGCCTGATATTATAACTAAACCGAGAAAGAACTGCAGCGCATGCAAAT
GTCCTACCCTCATCATTTCCATTTCAGAGAGTCTCAGGTTGGATTAAAAAAAAGAGAGAGAGAGAGTCAA
ATTGCTTTTGAAAAGGGACATCCAAGTCAGTGGGATGTGAGGTCACCTACATTGCTTCATGTCCCACCTC
TCCCTCCATTCTCAAAGATGGTGGCTTAGCATCTTGAAATCTATCAGCTCACCCTCTCTCCTCTCTCATG
AAGACTTTTGTCATTATACACACATGCACACACACACACACGCACATACGCAGCCATGCATCTTGAATAA
GCTCCCAATTTAACATCATAGTCACGCCTTCAAATTTCATTTTGCTACATAAGCTAATATAGTCAAAGGT
TACACCTGTAACCCCAGCATTTGGGAAGTGGAGGCAAGAGGATCAGGAGTTCAAGGCCAGCCTTGGCTAC
ATAGCAAGCCTGTGCTACGTCAAACCTTGACTCTAAAACTAAATAAATTACCACAAAACCAAAATCCACA
AAAATAAACATTAAAACACACAGAAAGTTTGAAAATCAAGGGGCAGAAGATTACCAGGAGACCAAGATGT
AAAGATGATTTATCCATTTTTAATAACAAGTACAAGTGCCTATAACAAAAAAGAATGCTAGACTGTGGTA
TATTTGTAATATTTAAAAATGAATGAGACTCATAGCTACAATGTACCCAGAACAGAGCCCACAAAATCCA
GCAAGGAAACAGTGGCAGATGTGACCAGGTGGATGAGAGTATGTATCTCTGAATGTCACTGTCATTTTGT
CTCATGAAGCTCAGAGTCACAGCCAGCAGCCAGAGCATGTCCATTGCGGTTCATTTAAATGCAATTTGAA
AAAAAAATCCCGGGACAGACTCTAATTGGCTCTGCTGGGTCACATGTGGCCCTCCCAGTGCAGGACACCA
TACTGGTATTGTCTTCTTGAATCATGGGTCCAACAGGGACCCCTCTAGAACTGTTCTTACAAACAAGGTG
GGTGGATAGGGCAATCCAGACCTGCTGTGTTAGGAAGGTGAACAAGTGAAAGAGACGCCAAAGAAGAGGT
GCGGTCCTTGGTGCCGTGGGAGACTCCTTGGGTGTCATGGTAAGCAGATTGTTTGAGCAAGGAGCAAAAT
GAAAGGGGTTCGCCCCCTCTGCACTCTGCAGTCACAGCAGTTCTAGGGAGCCACCAACAAAACCACCACC
TTAGAGGAAACCGGAAGTGAGGCTTAGAACACTTCTCAATTGGCTACAGCTCAGCAGTCAGCCAGACGGC
TGGCTACGGCGTAATTAATTGGTTAACTGCAATGCCCCTCCCCTCCAAATGTTATTTAACATCCTTTGCT
GCGGAGAAAAGGTCCCTCCAGAAAGAGGGCAGTCAGGTACAGGCAAGGGATCTTTCTGAACTGGACTCAC
ACCTGTTTCTTATCGCCGCTGTAACAAATGATCCCAGCCATGATGCTTAAACAAGGACTTACTGTAGGGA
AAGCCTTGGAAAATGTGGTGCCAAGTGATTTCCTCACCTTCTTCCACTTCTACAGTCACCTACATTCCTC
CATCCTCAAAGATGGCAGCTTAGTGTCTTGAAATCTCTCCCCTCCCCCTTCTCCTTCTTATCTCATAAAG
ACCCTTGGAATTATATTGCCCACCGCTCTCCATGGATCTTGGATATGCTCCTAATTTAAAATCCTATCCA
CACCTTCAAATTTCCTTTTTGCACATAAGCTACTACTATGTTCAAAGGTTCTGAGGGCCCCCTTCTAAAT
AGGGGAAGACATTGTTTTGTCCAATTTGGGGTTACTCAAACTAAAGTCAGTAGAAGGCATTCAACACATA
TATTAAGATATAGCACAAGGAATTGGCTCACACAATATTGGAGGCAAGCAGTCCAATCACAAACAGGCTC
TTAGGATTCACCAACTTGGGACAAAGCTGCTGTCCGTGGTGTGTCTGTGTGTGTCTCTGTGTGTGTGTGT
CTCCTGCACACACTTGTCAGTTCTCTTAAGTTTTGTGGTTGATTGGCTTGGGCCAACTCAGATCATACAG
GATCATATAGATCATCTTCCTTCCTTCAAGTCAGGCGATTCTGCATTTTAATTACATCTGCAGACCTCGA
TTGTGTTTGACTAGCCAGAGGCGAGGCTAACCATGCTGGCCCATCCAGTGTACTGTTACAATTAGGACCT
GTTGGGGATAGACTGTTGCAGCTCCAGGAGTTGGGGCTACCATGGGAACAGAGGGAATCCCCAGAAGGTA
GCGCCTTCTTGAAACTGTTGATTTGAAGAATGCATGCTTGGGGTTGGGGATTTAGCTCAGTGGTAAAGTG
CTTGCCTAGCAAGCCCAAGGCCCTGGGTTCGGTCCTCAGCTCCGGAAAAAAGAAAAAAAAAAAAAAAAAA
AAAAAAAAAAAAAAACTTTAAAAAAAAAAAAAAAAAAAAAAAGAAGAATGCATGCTTGAGTGAACTGTAA
GAAGCCCAGGAGGGGGTGAGCACCTTCCCACAGTTTGAAAAAGCTGTGTCGAGAGAGAGAGAGAGAGAGA
GAGAGAGAGAGAGAGAGAGAGAGAGAGAGAGAGAGAGTCTGCTTCCTTCTACACCAACTTTGCCAGGCCA
CACAGTGAGCGAGCTACCTGTGACTACAGAGATGCCCCTTCCTTCTGTTGCCTTCCCATTGCTAGGTCGC
AGGCCCCTGGGGTGCAGACTTGAATGGGCGGGTGGTGGAAGGCCGGTGAGCTCATGTCTGCTGCTCCTGG
AACAGGAGGCAGCTGTAGGCGTGAGTGTTTGCTTGGCTTATCTTTTCCCATCTGAAGATGGTCATTGGTG
ATTCATTCCGGAATCTTGGAGTCTGCGGGGAGAAGTCCCAACCCAAACCAACCAGGTGCAGGTAAAGGGA
CACCCATCCCCCTGTGTCCCTGCACTTGCCTTCTAGTGTTCTAAGAGAAAAAAATCGGCCTGGGGAGGGG
GCTCCCCAACACCCACTGTGCAGGCAGACTCTCAAGAGGATTCCCCATGCCCAGATAGATAGCCCAGAAG
CGGGAGAGAAATCTGTAAGTCCCAGCTTCTCACTCCATGAGAAGTTTACATTTTAGCAACTCTGTTCACA
AAATGTGTTTGTCCAGAGGTTGAGCTTTGTCACTGGGAAAAAAAAGTTGTCAGGGAAGAAGGACTTAAGG
GCTTGGGAGTTCTTCCTTTGACTCGACAGCCATGATTGTCAAAATGGAGTCTATGAGCTGGTGGCACCCT
CCACACAAGCCAGCTTATTGTAAAGACAAATTTTCATACCCCATCCCTGACTACTGAATGACTGATGGAA
AGACTTCTCGGGTACTTCTGATGTGCTCCAGCGTTCAGTGACATCTCTCGGGCAAGCACATCTTGAGCCC
CTGTTGTGCATGCAGAGTGTGCTATGGTTGCTGCTATGAATGTGGTGGTGAAAGTGAACAGCAAGAAGTA
ATTGGTATTCAACTCCCACGGTTGCCAGGTAACTCGACTGGCTGATTGTAATGGATGGAGGAAATTTGCC
TGGCCTCTGAGGTTGATGAGTAAAGGCCTCCCAGAACTTTCTGGTGTGGCTCAGGTAAAGGCCAAGGACT
GTAATCTCCTGTCCCAGAGACCTCTCTGTGCAGGGAGACTGGAGTGGGACTGTATTAAAGCAGAACCTCC
TTAGCCAGGAGGAACAGCCATGTTGCAATCTGCAGGCTTTCTGGGGGGGGGGAGGGGCTCAAAACACACA
GGCTGGGTTCCATAGCAACCTCATTTAAGCCATGGGAAAGGACCAATTGTCTGGGAGGCTGAGGTCAGGA
GTTCAAGTTAAATAGTCAAGGTAAGCAGGGATCTGAGAAATGGGCGTTCTCTTCTCTGTATCTACTCCTG
GCTGCAGACAGTTTGTGGTGCTAAGAATGCCTGGTGCTCTGAATGAGCCATTCCAGAGATTGTGGGAATC
CCTATGGCCATTCCCTTGATGCTCTTTGGGGCCGTGGTGTGAGTGTGGGTTGGGAAGAAGCACCAGGGGG
TTGTGGAGACCAGGGTGCCATATTTTTGTGCATGTTACGAAACTCACATCTTCCCGCCCTCCTTTCTTGA
CCTCTCCATTTGTCTTTTATTTTCTTCTGTCACAACAGCCTAGGACCTGATGAAGCTTTTCTGGGAAAGA
ACTTGGTCTCAGTTCCGTGGTCTGACTGAAGACCCCTCCCTTTGAGGTTTCATTCAGTTCTGTCATGCTC
TCTCTGTGTTGACCTTGAAAATATTGCTTTCTCTTTTTCTGTTGCTCCATAGCTCCCAGCAAAAAGTAAC
CCACAGTTAAGAAAAGGTTTGGCTTGGCTTATTCTGGATTGAGGGTACAGACTGGCATGGCAGGGCTGGC
ATGGTGGGAGTGTGCAGGAGCTGGCTTTATGCACCTACAGTCAGGGAGCAGAGAGAAAGAGATAACCATC
AATGCTCCATTTTCACTCTGCTTTTGGTTTAGTCTGGGACCCCAGGTAATGGGAGAGAGCTACTTCCATC
CTTCATGGTCTTCTCTCTATAGCTCAACCTCCCTGAAAATGCATAGCGATTTGTCTCTTAGGCAATTCTA
AGTCTGGGCAAGTTGATAGCCAAGATTAAGAATGTGTGTGTGTGTGTGTGTGTGTGTGTGTGTGTGTGTG
TGTGTGTGTATGTATGTTTATATATATATGACATGGCAGCTTTGGGGGAGAGGGGCAAGTAGGGTCCATA
TTCTGAGAGTGAGCACAGGACTCCAGAGCACCGCTTGGCGACCAGCCCTCCTGAGAAAGCTTCTACTTCT
GGGGAGGATCACTTGTGCAGACTTCTACCCATGCAAAGACTAGACCAGAGACTCAGCTTGGAGCACATCC
CAAATAGCATTAGAGATCAAGTGGCCCTTGTCTGGCTGCAGATAACCACCTCACACATCAGAGTTTAGAT
GTGTTAGGGTGTCTGTCAAGTAACTGGGGCTTTGTCCTGTTTTTTTTTTTTTTTTTTTTTTTTTTTTTTT
TTTTTTTTTTTTTTTTTTTTGTGCCGCTGTAGCACCACAGCTGTCATGAGGCACCCAGGAGGCTCTAGGT
CAAGTAAGCCTTCCACCTTCTTCATGGGGAAAATGGAGTCTGCCAGCGAGGCCCCCCGGCTGCGTCGAGC
AGGAGTAAAAGCTAATAAAGATAAAGGAATCAGTCATGGGTGGGGATATAGCTCATCCTAGTACGCACAG
GCTCCAGGGCTTGATTCCCAGCATTACCAGAAAAACAATGAAGAAGGAAGGAAGGAAAACAGAATGGAGA
AAAGATTTTTTCCCTCCAGCTGGAACGCGCTCAAGGGGCTTCAGTCCTACGGAGCCCAAGAGGGACTTAT
TTCAATGCAGCCCTGGGTTCCTGTTGTCCACTGGGCTTGAGAATTGCATTCCACACTATCCCCCCACCAG
AATGAGAAGTTGAATAATGAGCAGAATTGAAGTCCTCTTGCCCTGGAACCAGTTGCTGGCCTGGCCGGGA
CCTAGAGGTCACTCCCCGTCAGCCCTGAGCCATCCATTCTCCAATCGTGCCACCAACCATTGACAAGACC
AGAGAGAGCTGGTCTTGTCCTTGGTAGGCAGTGGTGCCTGCCCAGCTTTCCTCCCATGCCAGGCCTCTCA
CCGATGCTTGCTTTCTTTGCCACCCCTGTTCCCTCCTTTCATTCTCTCTACGGGTCCCTCTCCCCTGACC
CATCAGCACACTGACCTCTGCGCACCCGCTCCCACCTGTTCCCCCGCAGCCCATTGGCTCCTTCCTCTCA
CCACCTGCCCTGCCCTGCCCTGCCCATGTTCTGACCTCCTCACTTCCCTCACACCACTCTCTCCGGTGCC
CCTTACCACCCCTGGGCCACCTCAGCTCTCAGGCCTTCCCTTGCCAGAATTCCTCTCCCTTGGCTGCTCG
CTCGTCCTGGCCGTTGTCCAACTAGAGACTTCCTGCCTTGTCTTCGCAACCTCGCCTGACTGCTTTCCTG
CGTTCTGCCCTGTTACATGACAACCTTTGCAGGAGGTCCAGCATGTGTCATACCTGCTTAGAAGTCTTCA
GTGATGACCAGATGGAGATGAACTCCTATGGCTCCCCAGGGAGGCAATTGTCACTCACAGTGTAATTTCT
TCCTGCCACACCCCTAGAATGTTCCCCAGATTCCTCTGACCCTATCACCGTGTGACAGATGCGCAGCCTG
GCTTCCCTGCCACACAGTCACCTACAGGGAAACCTCCTCCATGCCTTTGTCACTTCATTGGTAAAGTCAG
ACCATCAATGGCAAGCGCATCTCATGAACCACCAGAGGTCATGTGGTAGAACTGCGCTCACAGCCTAAGG
CAGAGAAGCATTTCCTTCTCCGGAGGTGTCTGATGCAATGTGGAGATCTGAATGTTGGGTAAGGGCTTCT
GTCTCTTCCTAGCATCCTCTTCCTTGCCCAGGGGAGAGAGGGGGCAATCTGCTCTGGCTTGATGAACCTG
CTCTGCACAGGACCAAGCTTTCAGGGGACTGATGTCATGTTGAGTAATGGGAGAACGTGTGGAGACTGCC
GCTAGTTCTGTGGATCCTCTGGCAGGCTGGGAAGGGGGATATGAGGTGACCATGATAGAGCATGTTGTGT
CCCCATGTCAGAACAAGTGTGTCTGGTTGTGAGTACTGATTATGGCGAGGATCCTGACCTGTGATCAGTG
AGTGGGGGGAGTGCTCACCTGTGGGTATGGATGTATAGACTCTGCAACACTGTCACCTGGAGAGGGACCT
TTACTTAGGATCAGCCAGAGGAGACAGTGTTGACAGTGAGTGGGGCCAGCAATCCACTCAGTGGAGGTAG
GCAGGTGTCTGCACACACACACACACACACACACACACACACACACACACACAGGGTCCTCAGGTCCTCT
CCTCCATGTTTCTTGTACATCTCATTGGGGACCTACTGAAGCTTTGTTCTCAGACCTCCTCTGTGGCTTC
CAGCAAGTATGTTGCAGTTTTTCGGAAGGTCTCCTGGGAGCTGCTGTTGATGAATGAGTGGTGGGTGGAC
ACAGGCTGCCCTGGGCAGGGGTTTTCCTGAATGAAAGTTCTTTCCTGTCCCACCAGGGCGCTGACTCATG
TCTGATGGGACTTGCTCCGCTCTTGCCTGGGTACTCCAGGGGCACAGCTTAACACTCTGTTTCTCATGCC
CAGAAAAAAAAAATTGACAGCCCTTTGTCTGTCTGCCAAATTAAACCCACAATAAAGACCAAAAGTCAAA
ACTGAGTCCAAGGCTTCACTTCTCCATAGGGAGATGAGTTTGTTGGGAGCTCTGGGCTAGAAAATGACTG
TGGGATTCTGAAGAGACTGAAAGAACCGATCCACAAGAATTGATGACAAAGAAATTTGTCTTTGTCTCTA
GAACTTTTAGGGGGAGGCATGGCTACAGGAAAAGGAAACCACCTGCTTCTACCGTGAATTTACAACCTAA
TTTGACAACTTTGTATTTGTATGCATTTAATGCTAGCTTTTTGGTGTCTGGATAAGAGTGGGCCCCTCAC
TGACAGTCACTCCTTGCCTTATCCATGGTCTTTAGTGTTCCTGGAGAGGATCGCTTCTTCCTCATGCTTA
CCCTGCCTCTGTGGTCATTTCCTTCGCGCCCTGCTGCTGTGTGCTGGATGCTGAGAATGGCTAGGGCCCT
GAGCCCCAGTGATTGAAATCACACTCTGGGCTCAGTTGCCTGAAGGAACAATGGAGAGCTGTAGCTCAAG
GGGCCGTCCTTGACACTAGCCTGCTGAGTTCATGAAGAAAGGAGCTGGAGGCTGGGGGGCAGATCAGGCG
GTGAAGAGTTTGTCATGTTAAGCATGAGGAGCTGAGTTCAATCCCCAGAGTCCACACTAGAAAATAACAA
GGATTGAGGAATCTGGAGGGGGCAGGGACTCCACAGGAAGACCAACAGAGTCAACTAACCTGGACCCTTG
GGGGCTCTCAGAGACTGAACCACCAACCAACGAGCGTACCCAGGCTGGACTATCTCCATCCCCTGCATAG
TTGTAGCAGCTGTGCAGTTCCCCCAACAACTGGAGCAGGGACTTACCCTGACTCTGTTGCCTGCCTGTGG
ATCCTGTTCCCCTAATTGGGCTGCCTTGTCTGGCCTCAGTGGGAGAGGATGTGCCTAGTCCTGCAGTGAC
TTGAGATGCCCGGGTGGGTTGGCACCCAGTGGAGGACTTCTTTCTCAAAGGTGAAAAGGAGGCGTGGGGG
AAGGGGCCGTGTGAAGGTAGGACTGGGATGATGGGGGGCTGTGATCAGGGTGTAAAGTGAGTAAATAAAT
AAATTTAAAAAAAAAAACAGTAACAAGGCCAGGCATGGGGCACATGCCTTTAATCCCAGCACTCGGGAGG
AAGAGGCAGGCGGATTTCTGAGTTCGAGGCCAGCCTGGTCTACAAAGAGTTCCAGGACAGCCAGGGCTAT
ACAGAGAAACCCTGTCTCGAAAAAAACAAAACCAAAACCAACCAACCAAACAAACAAAAAAACAAACAAA
AAAACCCCAAAAAACAAAAAATAATAACAAAACCGAGTGTGGATGTGTGGGGTTCTTGTTATCCCAGCAC
TGAGTGGGAAAGAGCGCAGACGGGAATCCAGAGGCTTGCTGGCCAATCAGCTCATCCTACTTCTCAACTC
CAGGTCAGTGGAAGACTCCGTCTCAAAGAAGAGGATGGCAGCTGTCTTAGTTAGGGTTTCGTTGCTGTGA
AAAGATACCATGACCAAGGCAACTCTTATAAGGGACATTTATCACCACAGCAACTGAGGTTGTTTTCTGG
CCTCCAGACACAGACATGCATACGTATACACAGAGAGAGAGAGACAGACAGACAGACAGAGAGGCTTGTA
CACAAATGTATACCACATGCACATACACACACGGGCGGTGGCAGGGAAGAGGAAGAGGGAGAGAGGCACA
CATGCAAACACATGCACATCACGTGCATACTACATGTGCGCGTGCACACACACACACACACACACACACA
AGGAAGGAAGAGCCGTCAAAGAAGAAAGCATTTCTCACCATCATTTGTTGCCCCAGCAACTTCACGCTTT
CTATTACGGACACACTTCGGCACTGGGTTGTGGCCACTGTGCTGGGAAGCTTAGGCAGAGGGGAAGGCAG
ATGAGAGTTTGAGTGTGGACAGCGCTGTAGGCTGTGGACTGGGCTCAGATTTTCATCCAAGATGGTGGGA
ACCCACAGGCTTGTCACTGTCACAGCAGGCTCTGGTCTCCATTTTCAATCATGCTGCCACTTAAAAGTCC
CTTCCCACAAAGCCCAAGGGCAAACTGACCCTAGCTCTTGGAACTCAGAAGAGTTTCTACCTTGGAGGTG
AGGCAGGTGGCAACTGAAGGGACCCAGAGACCTTTGGGGAAGCTGGTACTGATCTAGTTTTTATTATGGG
TCCTTGAACTGTAGCCAGCTCGGGAATCTTTAAGCTGTACTCTTACAACAGATGCATTTTCTGTGCGAAT
GTACTCCAGTAAGAGCTTTCTCAGAAGACTTCTCTGGCTGTTTAAAGAGTAGATCAGAGCAAGGAAAGAG
TAGGTATAGGAAATGAGAATAGGGATGTGTCTATGAGTCAGGGGATGTCACCAGCTGCTGGTGTCAGGGA
CTGGAGGGGACCAACATAGATGAAAAATGTAACAAAATTTGAGATTTGGCTTGGAGGCACCGAGGGCAAA
ACCTAGAATCCAGAAAACACTGTTGGAAGGCAGGAATGACGGGGCATGGACTTGATAGGAAATGGGTGGA
AATAGGAGAGGCACAGTTGAGAGGAAGTCAGCCTGGGGAGGGAGGGCTTCAGTTAAGCATCCATGCTCCC
ACAGGTGGGTAAACAAGACACCAAAGGTTGGTACATGTGAAAGCACTGATATACATCCCGGGGACCATCA
ACATCTGGGGGATAGAGAAGACATCTCTTATCAAGGTTTTACAATAGAAACAGACCTAGAGGAAGTGACA
TCATGTACTCCAGTGCCCATCCTTGTTGAATTACCTGGTACAGAGGTTCACATCTGTGATCCTGACTCTT
GACACTTGGGAGGTAGGAGTTTGAGGCTAGCCTGGGCTATATAGCTAGACCCTATCTCAGACAGAGAGAG
TGACAGAGACAGAGACACAAGAGAGAGACGGGTAGGAGGTGGGATAGAAAGGAGAGATGAGGTGGGGGAG
TCCTATAGGACTTAGATACTTTTTGGAAAGCCTTATGTGATCGCCAGGTACTGTCAACTTCAGCAAAAAC
AATCTCTGTAGCACCTCCTTCCTCTGAGCCCTCAGAAGAGTCAACATTGTCACACAATGGAAGAGTAGTA
TGACTTAGTGTCTGTTGGCTGTTGTCATTTGCCAACTACAATATGAGATGAGCTTGCAAGTTGCCCTGGG
CCTTCGTCACCTTCTCCACAGGGTTATGTGAGATTATCTAGGTTTGCAAGCATTTACTGAACATGTACCA
TGCATTGGCTTACTGATGTGACAGTGAGAGCATGCCTGGAAAAAAAAAAGCAATGGAAACAGATAATAGA
GACAAAGATGATGTGACGGCCCCACATGCCCTCCCTCAGAGCTCCCTAGCGGTCATTGCCCACTGATAGG
ACCATAGGACCCCTCCTTGGAGGGACTCTTGTGAGACTGTCTTTGTATTATAGTTTGCACTTTCTCTATC
TGAAAAACTGGCTTCCCTTTGCTTGAAGGCAGGAAGGGATGGGCTTTATCTTCAAATCCTTCCAGGATGT
GAGAAAAAATATTGACATCCCATCTTGTTACCAGAACAAAGACGAGGAAGGATATAAAATAGCAACGTTT
CCATATTCACAGGGTCTAGATGTCCCTTCCCTGCCATAAATGCATTCAAGGCACGTTGGACAACGGCGGC
TTCCTTTCAACCCAACAGGTGACTGCGAGCCAACTTCCCCCAGCACAGGATGGTGCTTGTTCTTCATTAA
GGAGTGTTTTCACTGTCTTAAAAGACAACAAAAGCTTTTCAGAATCTCTGGAGAAGAATTCAAGCATATA
TTGACATGAACACTAGGGATAATTTTATTTCCAAGTGTCTATTTGCCCAGGAAAACGTTCTTCTAGTGAA
TATGATAAATCCATAGAGTCTCACATGCGTGCATTTAACCTCCCCTCCCCCAGGCGATCTCTCAGCTTGC
TCTTCCATGACACCTCGTCACCACCTCCTTGCAGTGGTCTGCCTCACACACTCCTGGGGCTGAGCACCAA
GCACAAAGCCCCACCCCCACACCGGGATGGGGGAACCAGAGCATCTCGGAACATCCAGTGCTGGACGCCT
TTTAGGGTTCCTAGTGTGGTTTGGGGTCCAGGATTCCCACTGCTTCTCTCCTTTGTGCCCCAGATTTAAA
ATGAGGATGCAGCATGGAAAGCCACAGGCTTCCTGTCCACAGTGAAGTTCTACATTTTCTTTTACTTCAA
AGGACACCCCAGGACCCCATCTTATCAAAGATCCTTTTTAGTGAGATTATAATCCCAGATCCAGATTCCC
AAATTGCCTTCTTCCTTCCCTATGAAGAGACAGGAGGTGTCTTATATCTTCCGTGCTCCTCTTTAGACTA
CTCTGGTACTTTCTTTTTTCTCTTTTCTTTTTTCTTTTCTTTTCTTTTCTTTTCTTTTCTTTTCTTTTCT
TTTCTTTCCTTGTCTTGTCTTGCTTTTCTTTTGTCTTGTCTTTCTTTCTTTCTTTTACTTTCTTTCTTTT
CCTTCTTCCTTCCTTCCTCCCTCCCCCCCCCTCTTTCTTTCTTTCTTTCTTTCTTTCTTTCTTTCTTTCT
TTCTTTCTTTCTTTCTTTCTCTCTTTCTTTCTTTCTCTTCTAAGAACCATCAGGACACAGTGCTTTTCTA
GTTCCTAACAATGCTAATGTTCCTGATGCTACCTTGGCTCATTTCTCCTCCAAAGGACATCAACTTTGAG
CCCAGGCTGGCTATTTTGAATCTGAGTGTTATCTTGTGTAGACATTCTTCCTGCATGGGAATATACTTGA
TTTCCTGAATGATTGTGTGACAGAGACACTCCCCACGGGGGCACTGGGCTATGCCCGGCAGAAGGTACAG
GAGGCTTAGGCAGGCTCCCTGGCTGTTCCAGGCCTTTCTCCCTAGGTCACATCTTTGTTTTACATCCCAC
TGTCCCTTTGGAAAGACAGTAATAAGGATTCACCATCCATCTTCCAATTCTCTGGGCCCTCAGGGGGGTC
TCAGAGGCACCAACTCAGTCTCTCTCTCTCTCTCTCTCTCTCTCTCTCTCTCTCTGAAATGTCAGATGAC
TCTGGAAAACATCTTTATCTGTGTTGAATAGGGTACCTTGAACCTAGGACCTCACCGTTCAGCACTTAAC
CCCTGGTCCACTGCCGGCCAGCAAGGAGGATGAGCTGTGGGCAGGGAAGTTACGTGGGAAGGAGCACACA
CAGAGTCAGAATCACCATCAGGCCGAGTGCCTGCAATCAGAGGCACACCTTGCTCTGCAAGACTCAGCTA
GCGAGATAACAGTGTCATAACCCGAATGTGCTAGGGTTATGAGGGAGGATCTGTTTGTACTTTGGGATTG
ACAGTTTAGTCCACTGCCCAACATATAATAAATGGTCTCTTAATGGTGTTCTTGACACCACTGTGATTTG
AAAAAAGCACACCCATACCACCACGCTGTCCTCCAATTTCAGTAGTGGTCAGTAAACTATATGAGGTAGA
GCCAACAGTTTGTTATGAAATAGGCTTTCTCTTAAATGATTCTGCTCCATGATAGGCTGATATAAATGTC
TTACCTGGCAAGTGGGAGAAACATAGACATGTAGAGCATTTTCTATTTATGAGAAGTTTATTAAATATGC
TTCTTGGAGCCCTTCTTTATTCATTGAGAAAGTAGGGGCACAAGGATGGGAATAGATTGGGTTTGTAATT
CAAGACGGTAGACTAAACACATGCTCATTTCTCCTCAGCTCCAACATCCCCATTATGATGAAAACGGTGA
TTAAGGATGATTAAAAAAAAGGACCAGAAACAGTTAAAAAGGGGGACACACTATGAATGGATGAAAGAAC
TCAATAATTTCTGTGGCATGGACAATGGTTAGTGGATGGGTCAGACTGGGAGTCACAGACTCTCAGGTCC
CTTCAAGGGGTGTTCAGTGGTGGGAAACCTGAAGCCTGCTGCAACTCCAGAGGCAGCAGGGATAGAATCT
GTGAGTATTATGAGGTAGAAAGCTGAATTCTTGGGGTGATAGTCAGCAGAGGACAGCCCTTTGCCCAGGG
GAAAACCCAAGCGTCCCTTCATTGTGAACCAAGGGCACTATCACCAGGGGGTTTGGGGCACTGGTTCCTG
TGGCTTCCAGTTTGTTTGCCACAGAGTGGGGTGGTCTGATAATAATAAAAGACCGGCAGAACTGTGAACA
GCTGGGAGGAACAGGGAGAAGGACACATCTAGGTAGCCAAACCCCTATCCACGATGGTGCATGCTCTAAA
ACGGCAGAGCAAGACGTCCTAGAACAAGCATAGCGGCTTGAGATGTGTGATCAGCAGAAAGCCTGTTCCG
TGGACCTGCTAGAGCGGCTAAAGCTGCTTGCCCATAGGTTTGCTACTCTGAGCCCTTCTGTGCTGCTTGA
GGTTTTTTTGTTTTTGTTTTTGTTTTTTAAATTTACACGTTGTTGTATAGATGATAAGATGAAGGGGGAA
AACAAAACATGTGAAAAAAATGTAAACAGATTTTAATGTTCTGTGTGGAATCTGGTTGACAGAGGCCAGG
AGTTCCTTTGGGCTAGAGCTGCGATTGGAAACCCATCTGGTCAAGAATTCCGTTCCTCTACACAGTAGTT
AGTATTCCTGACAGCCTAGGCTGTCGTTAGCTCCAGCTTAGAGATGGAAGAGGAAGAGAATGGTCTCTGA
GCCCAGGGGTCTATGCTGTTGCTGTTGCTGTTAGGATAGCATCTTTCTCTAGGATCTGGGTCTCCTCCAC
TAGGCTGTCTAGCCAGCAAGCTCCAGGAATCCTCCCACCTCTGCCATCGCAGTACTGAGGTTTCAAGTGC
ACACCACTACACCCAGTTCTTTGGGGGTTTTCCCACAGGTTCTGTGGTCTGATTGATTGCTATAAGAAAC
ATCTCACAGACCGAGATATCTCCCAAGTCCTCTCTGAGGTCAGAGAAACTAAAGCAGGCAGCAGAGGACT
CAGAGCAGCCCGGGCATACCCAGAAACACCCAACACTCATGTGGCATCAGAGGAATGTTTCTTTATGTCT
GTGGCTTTTCCTTCTTCTCTCCTTCCCTCCCTCCTTCCCTCCTTCCCTCCCTCCTTTGGGAAGGACTGCT
TCTTCTAGACCACAGACAGCTGCCATCTAGAAACACCGGAAGGCCATCAGCTGATGCCCTGTCAGGCCAC
TTAAAGCTTTTTATCATGCTAGCCAGCTCCAGTCTGAATTATCCCAGTGAAATCACATCCTAGTGGCCAT
GAGGGCTCATGAAGGGAAAGTCCTCCCTCCCTGAGAGCTGTGTTCACAAAGTCAGAGCTGGGTGAGAGCA
GGAGCAGCCATTACCTGGGGGTCCGTGCGTCAGACCTGCCTGGCAGCTTCAGGGCCAGCCAGTGCTTCTG
TGGTCTAATGCCAGTTAGCTATAGCTCCAATGTCTAGACTAGGAAAAAGAACGAGTCAAGAACATGACAA
TGAAAACTGACCAAACAAAACTGTGTCCCAGGTGTCCTTGACCCTTCCCCATTTATCCTGAGTTCTCAGC
AGTCCCTCACCCTGAGTGCTTATGGTACAGTTAGTTGTCCTCCGCATGCCACTGAAACTTGCCTTGGGCC
CCAGACCTCTCTCTATCATCATGTGCATCCTCTCAGCCTTCCTTTGGTCTTTTTTAAAAAATATTTTTAT
TAGGTATTTTCCTCATTTACATTTCCAATGCTATCCCAAAAGTCCCCCATACCCTCCTCCCCTTCCTTTG
GTCTTTAAAGGTCACTTCAATTTCTCTGCTTTTTTTCTCCTTTACTCTCCCTGCCACGCCATCCTCATTT
GTTAGGACTGAGGAATGGAGCTGTGAGCATGACCTGCACATTTGGATGTCTGAGAGTCTCCCTCATATCC
AACCCTGCAAGAAAGGTCCCCAAAAGGGAAGGCCTTTGCTAACTTCCTCTGTGAGATGTACCTGTAAGAG
TCTGTCTCTCTGTCTCTGTCTCTGTCTCTGTCTCTGTCTCTGTCTCTGTCTCTCTCTCTCTCTCTCTCTC
TCTCTCTCTCCCTCCTTCCCTCCCTTTTTGGAATAGCATGCACACACAAATCTAAATATTTACACGGGTT
TTGAACTTTTCAGCATAGAGCCCTTGAGTAAACAAACACTCTCACAATGTTGTCAATGCTCATACTTTAA
GACAACAACAAAACCAGAAAACTTTGCGGAATGCCCTTCAAAGGGTCAGGATATTATTTTGAAAACCTTC
ATGCCTCTGACTCTGTCCTCAAGGGAATTTTCCTTTAGTCAATTTTATTGAGGTCTACTTTGCTTATAAT
AAAATGCACACACTTTAAGTACAGACTCTACCTGTATAAATATCACTGCTATAAAAATGTAGACGACATC
AACCACATCAGAAAGTCCCTGTATTCATAGTTGCAAATTTTTAACTTTTGTGGTTTGGGTTTGGCATTCT
CAGGAGCCATTCTGAGTGAGACAAGTAAGCCAGGGGGATAGCTGAACCGTGCTTACAAATTATAGCCACG
TCAAAGGGGAATTTTATACATGACAGAGACTGATCATTGCAATGAACCTGCAGTACCCTGGCTTTTCTGA
CGGCTGAAGCCTCTAGATCCCAGGTTATTGTGACATTTTGAAAACACGATGCTCACCTGCTTCCTCTGAG
CCAGATATTGCTTCAAGCCCTGGGGGATAATTTGTTCTAAAACTCTCCAGTCATGTTTTCTCCTGTAAGT
CTAGAGCCTGTTGAGGAAATATCAATAAAGAAGAAATCTTGTGTGTTGTACAATCTATAGAAGCAAGAAG
TGCCTGCGAACAGAATGAGAATAGAGGGAGGAGATGGAAAGTGGCTGACCGTAGTAATCCTCGGACTGCG
TCTTTACCTTTCCTTGTCTTTCTGTGAAGCATTTTGGAGTGTTTTGTTGTTGTTGTTGTTGTTGTTGTTA
AATGGAGTTGAGTGGAACACCTAGCTTCTTATAGGTAAAGGGTGGGCCAGGTTCTGGGGACCTCAAGCAA
GTAAAGAACTTGCCTCCCAAGCAGAAGGGCCAGAGTGCAATCTCCAGCATCCATGTAAAAAACCATGTGT
GGTGCTATGTGCTCGTAAGTCCAGTGCTGAGTTGTAGACACAGTGGATCCCTGGAGTGGATCCCCATGGC
CAGCAAGCCTTGCCTAGTCCGCCAGCTCCAGATCCCGGAGAGAGTCGCTGTCTCAAAAAAACAAGGCAGA
TGGCTCCTGAGGGGTTGACTTCTGCTCTTCATGTGTTATGTGCATAGACACACACACACACACACACACA
CACACACACACATAGGGGGAATTGGCTTAAACAGAGCTGAGCAGGTCTTTAATGAAGTCTTTGACGTACT
AACATAATTGCAGCTTTTCCTGAGGGAAGGCTGTAGGACGCACCAATCTCCAGATATTTTTCTCCTGTAA
ATCCCACTGGATTTGGCGTGTGTGGAGCAAGGAGTCATTTTTACAGCTCCCTGAATGTAGCACATGCAGC
TTCCGCTTCTCATGGCACAGAGCTGAAAGCCACAGAGAAGTTCATGACTAGGGGCAAGGAAGGGAATGAG
GTTGGGCAGCTTGTGGGTCCAGCATGGGGCCAAGAATCCCAAACACAGCCCTTGGGTAATCTTTTTGACT
AGGGTGTTACAGCTCCTAGGTACTGTGGCTCAGAGTGACCCCATTGGTAGCCAAAGATTCCATGCAGAGC
CAGGATCCGAACCTAGGCCTGTTTTACTCCCAAACCCAAACCTGCCTACACCAGGATGGCTGTCCTAAGT
GAAGTATAGAGAGGCTGTAACTCAGCAACATGCCCCCACTTCTCCACAGTTAATCTACATGTCCCTTCAA
GCCACATACTCTTCAGGGGGCATTTACCACTTTTTCCCCCCACTGGCAGGTGATCGTTTTTTCTTCTCCT
GCTAGTTATGAGTGAGTATTTGGTAAGTCAGAGCAGACAAGGACACGGTCTCCTGGTTCTGTCCTCATGA
AATGGTATGCAGGGCCAGATGCCTGTGACCCTGGGAGTGGCCCCTTGCAGCAAGCTCTGCCAACCTACCC
CTCCATGGCTTCCTCTTTCCCCTGCATTTTCTCCTGTTGTCATAGCCGCACATCCCACCTGTCTTTATCT
GCTGCATCCCCTGGAGGATGGGGCTCAGACCCGCATCAGTACCTCTGTCCCAGCATCCATGAGGTACCCA
GTCTGGTCACAGCTGCAGTGGCTACTGCTGACGTGGGCATTGCCGCAAAGGGACATGGCAAAGAGATATA
GCAGGGTCCTCACTGTAGCCCAGGGGTGAAGATGTGGAAATCCAGCACTTACTGGAAATAGCAAAGGAGT
ATCTCTCAGGAAAGGCTCTGAGGCAGTTTGTCTCCCACCATCCCCGTCTTCCTCTCAGGAGACAACAGAG
GAAGTTGGTAGGAATTACTCACGTCTGCTAATTCATTACTAATTGAAAGGAAGGGAGATGGGTCTTCTCT
AAGCTCAACGCTCAGAAGCTAAAGTATACTAACTCTGACCTTGGGTCCCCTTTTCACGTCTGGCCTTGCC
TCCTTGGCTTCTGTATTTTATTCCATTTAGAGTTGCTGACCACTCAACTTCTGTGGGAGTCTTTGATCTG
GTCCCTTTTCACATATCCTGCAAGACGTCCCTGGCATCACCAATACCATCAGCGACACCTTTATCCTATC
TTCACTAAAATTCACTGGCTTAGTTAGCATCACCTGTCAACTTGACATAGCCTAGAATCATCCTCCCCCA
GCACCCAAAAAAGAATATTTGTTGAGTAATTGTCTTTATGGAGTTGATCTGTGGGCATATCTGTGAGGGC
TTATCTTGATTGTGAATTAATACAGAGAGCTCACCCCATCATGATGGCACCATTCCCCGGGAAGGTGACC
CAGTGCTGTGTAAGGAAGCTAGCTCTATAAGAACAAGTCTGAGAAAGCCAGAGAACAATGCTTCTAATGA
CTTCTGCTTCAAATCCCTGTTTGAATTTCTGCAATGGCCTCAGTTTCTCCATTTTGGAAAACAAACTGGG
ACAAAAACTCATCTTCAAAGCTGATTTCATCATAATATATTTAAAAATAGGGGTTAGAGAGATGGCTCAG
CGGTTAAGAGCACTGGCTGCTCTTCCAGAGGTCCTGAGTTCAATTCCCAACAACCACCATGATGACTCAC
AACCATCTATAATGGGATCTGATGCCCTCTTCTGGTGTGTCTGAAGATAGCTACAGTGTACTCATATACA
AATTACATAACTTTAAAAATATTAAAAATATTCCAGCCAATGAAAGGAAACACAGAAGACACTTTCTGGG
AGGCAGGACTGAAGAGATTTCTCTGGCCAGCAGGTTCTCTGCTGCTTCTGGTGAGGAGGGAGCTGGTTGG
TGGGGCTCAGAGGCAGGCAGCAGTTTGGAGAGCTATCTATTTCTCTGAGTACGGTGGCCTTCTTGAATAT
AGAGGCCATGAGTACAGACGGACATGCAGTGAGATCTGGTCTCTGTCAAAGAAGCCTCGAGTTGGCCTTG
CCAAGGCTCTGTCCAAGCAGCTAGGTCATTCCACAGGCTGGAGGGAGGCCCCAGCAGCAAGACCAGCTCA
GTTTCGGAGGGAAATGACAATGAAGATGGAGACTTAGTTGCTCTTCTCAGACACCGGCCCTTCAGTCCAG
AGAATAACTTTAGCAGAGTATGAAACATAACAGCACCCTACCCTTCTCTGGAGGAGAAACACTACCCAAA
GGAAACCTTCCAAGCACAACTTAAGGATGTGGGTCTTGTGGCTTGGTGCCTGTTCATGCAGCAGCTGAGA
TCCCAGGGAGACACAGCCTGGAGCTGAGGACACCATCAAATTCTTCTTCCATATTGCAAGATGATATCTA
ACCCTGTCTAGTATGGAACTCACATTTGCTCAGTACACACAGGACAAGGCATGTGAGGCCAGCTGGGACA
CAGTGACAATTAACCTCTGGGCATTTCTCCAAGGCCTTGAGCATTCATCCAGAGGGGAATATGACCAGTC
AGAAATTTTCTTGGTAGGAGGTCAAGATAGCCCTGATAAAAGCCCAGTAAGACAGAATGAGGATATTCAT
TTAAGTAGATGGTAGGAGGACTCTGTCAAACCACCTTCCTAGAGACATAAGAACTTCCTGGGCGGGCTGG
AGAGATGGCTCAGTGGGTAAGAGCACCCGACTGCTCTTCCGAAAGTCCGGAGTTCAAATCCCAGCAACCA
CATGGTGGCTCACAACCATCTGTAATGAGATCTGACTCTCTCTTCTGGAGCATCTGAAGATAGCTACAGT
GTACTTACATATAATAAATAAATAAATCTTTAAAAAAAAAAAGAACTTCCTGGGCCTTGGAGGATGGAGG
TTATGAGTGAGGACATGCCAGGAGAAGTGGAGCATCCAAACTGAGGCCCAGATTGAGTAGTGAATGACCT
TCAGCCCTCTCATGGCACAGCAGGAAGACCAACTAGCAGTTCATGAGAAAATCACGATACGGTTAGGGAG
CAACTCAAGGTGGCAAAGGCCCTTGACACTTGTCTAAGGGGGGGACCTTGCTTGGGGATGGCATTTCCCA
TGGTTTTAGTGGGCTCTTTGTGACAACAGTTATAACTCATTAGGTAGAGTGCTTGCCCTGGTCTGCACAA
AGCCTTACGTTCAACCCCCAGCACAGCACAGAAACAGAATGTGGTAGCACAGGCCTGGGAGCTCAGCACA
GAGTAGAGGTAGAAGGGTCAGAAGTTCAAGGTCACCTTCAGCTTCAGAGAGAGTTTCAACCCAACTTGGG
AGACAAGAGACAGGGTCTCAAAAACCAAACCAAACCAACAACAAAACCTGGGAAATCTGGGAGTGACTAG
CACTTTCAGACCATTTGCCATGGGGTGGAATATGTGGTATCTGATAGAGTTTCACCCTCAGAACCATCCA
TGAAGTGTTTCCATGAAGAGACAGTGCCAACTGTCTCAGAGAGCCCTGGATGGAAGGCTGGGCAGGCACC
CGGGACACTGGGCAGAGACCTCACAAGCCTGTCATCTGTCCCCCAGGTCTTCGTGCTTTGTCATGGCCTC
CTACAGCTCTGCCAGTTGCTCTACAGTGCCTACTTCAAGAGCAGCCTCACCACTATCGAGAAGCGCTTTG
GGCTCTCCAGTTCTTCCTCCGGTCTCATCTCCAGTTTGAATGAGGTGAGTGGGAGGCCCTTTATTTTAAA
AGACTTTTAAAATCAGACTTGTGTATTGTATGTGTGAATGTTTTGCCTGTATATATATATATATATATAT
ATATATATATATATATATATATATGTATGTATGTGTGTATGCATGGATGCATAGCAGACAGATGTTTACT
GCCTGAGGAGATCAGGAGAAGGCATTGGATCTCCTGGAACTGGAGTTACAGATGGCTGTGAACCACTACG
TAGGTGTTGGGATATGAACCTGTGTCTACTGCTAGAGCAGCAAGTGCTATTAAATACTGACCATCTCTCC
AGCCCTGAGTGGGACGCTCTTGCCAGGTTAGGTCATTGTATGCACTGACCTAATCTCTGAGAGGTACATA
AGAGGGGCAGGGTTTACCTCCTAGTGCAGAAAACCACCCCTAAATTTTGGTCTTTGATTTACTATTGTCC
TCATAGACAACGGACGGCAAGGAAACAAACCTTTGCCATTTATGTCACCAAAGCCTGGCCCTGGCCCTTG
GATGTATCACAAGTCACCCACAGCTCTTGGTGAATCCAGACATGGCCTCTGGATTCTTCTCACACCATGT
TCTAGGCTCCGTGACTAAACAAATGAGATCTAGCATATAGGATAGACCCTCTCTTACAGCAGTCAAGAGG
AACCAAAACTATGATTCCCACACCTAAACTTTTATCTGCATCATTAAATGCTGGGAATAAAGCTGTGGTC
CAGGAACACATGGAGTTTGTAGGCTGCTGTTGCTGCTGGCGGTGGTGAGTGTGTGTGTGTGTGTGTGTGT
GTGTGTGTGTGTGTGTGTGTGTGTGTGGCCAGGGGTGTGTAAAGAGAGTCCAGCCTGGTGCCAGGTTAAG
TGAGGGCATCTGAGTGTGTGGTGAGTACAGACCTTCAGACTGAAGCTCCAATCTCAAAGCCTTCCTGGCC
CCAGGTGATATGAAAGGACACAGAATGCCTATGGTAGTTCAAAGGCAGGTGAGGGGCTAGAACAGAGAAG
CCATGGGTATCTTGGCCTTGGAAAGCAGCAGCTCCTATTATTCACTCTGACATGACATTGAAGGATGAAC
AGACATGGCGGGTTGGATCCCTAGATCCCTAGATGTTAACCCCTAGTCTTTAGAGCCCTTTGGTTTTGAG
ACACAGAAGGGCAAAGTGTGTTCATAATGGGCTTTATGTCAGATTGCAAATGTTTAACACCAGCGAGTCG
GTGTTAAGCTGTTTTAGAGGCACTCCGGGGGTGAGACCTCAAGTCCTCTCCTTGGCTAGAACAACTGCAG
CTTTGAGGTCTAGCAACCCATGGCTTCTGCAGGTGAGCGCAGGCCCACATGCCCCGACCTTTCTTCCTGA
TTTGTTTTGGACCCCTCCACCATCTGTCATGCACAGACCCTTTCGAGTCAGCTGACAGCTGAAATGGTCC
AACACTTTAGTCCACATACAAAAAAATAATAGAGATGCTAAAAGGAAAGAAATGAAACCCAAGAAGCTCA
AGAAGAAGCTTTTCCTCTCTCTGCAGAACCCGCTTCAGAATGTTCTAGAACCCTCAGGCTGGAGGGCTCT
CTGCTAAAGCCAGGCTGCCTCCGCATACACCTCACACACCCTCACAGCCTCCTGTGTGTGCCGCAGCCTC
TGTTTGTTGGTCATGCTCTTTGACACATGTCAATGAGCACCAACTTTGGGCCAGGCACTATCCTAGATGC
TAGGGAGGCAGAAAGGAACAGACAGACAGAAAGCTAAGACAAACATGTAGAAATAAATCTCTAGTAGGTA
GATGGTAGGCTCCATGAGAAGGTGGCATGGGAGAGGAAGAACATGCAGGGTCAAAGTGGTGGACACAAAG
GGACATCCCTTGTAAGGAGCACCAGGAACAGTGTTCCTGATGTCCACACTGAGCAGGACTTGGAAGTGGT
ATGGTCCTTGTCCTTTGTTTTGAAGAATTTAGCATGGGGATGGGGTGGGTTAGAGTTATTGAGGCAGAAG
TCCATTCTTCCAGAAGATTGTTCTAGGACAGTGTAGACAACCCAAAATGGGCCTCCGTGGTTCCGGTTAC
CTCCTGACTTCATGGATGGAGCTATGACACTTACTGTTTGACTGTCAAGTTCTAGTTTTCTTTCGGTTGC
TCTGATAAAGACCATGAACCAAAAGGAACTTGGAAAAGAGAGTATTTATCTCGCTTACACTTCCATCCTA
GGTCACCATCACAGGAAGCCAGGGCAGGGACTGAAGCGGGAGCAGAGGCAGAGATCACACAGGAAAGCTG
GCTTCCTCCTTCAGCTTGCTTAGATTGCTTTACAACCCAGGACCGCCTGACCAGGGACGGCATCACCCGC
AGTAGGCCAGGCCCTCCCACATTAATCATTAATCAAGGTAGATGTCCCACAGACTTGCCTACAGGCCAAC
TGATAGAGATATTTTCTTGATTGAAGTTTCCTCTGCCCAGATGGCCCGAGTTCGTGTCAAGTTGACAAAA
AAAATTAGCCAGCAGAAATTTGACATTGAGAGTAGTAACATGGTGTCTGGGGCCATGCACATTGCTTGGT
TGTTATTCTGACGTTCAGTCCAAAGTCCCTGAGTCCATTACTTTGGACCTCATTTAAGTTAGGAGTTTAA
TTTAAAAATATATGCTACACTAGCAGCCAGCCAGGACTGGAGCTTCCTTGGACTTTTGCCCCAAAAGACT
TTTCTAGAAGGGCTAGGGTAAAAGCTTAGTTGGTAAGGTGCTGATCATGCAACCACAAGGGCCCAAGTTG
TCCCATCCCTAGAACCTACATTTAAAAAACCAGGTGCAGTAGTGGCATGTGTCTATACTTCCAGAGTTGG
GGAGGTAGAGACAAGGGGATCCCTGGAGTCAACCAGCATAGACTATTTGCCTAGCCACTAAGAAACTGTC
TGAGAGAGAGAGAGAGAGAGAGAGAGAGCACAGAAGCACACCCACTTGCTAACACACACACCCTACACAC
ACCCACTTGCTAACACACACACCCTACACACACACACACACACACACACACTTAGGTTTGTGTATAGACA
TCCAATATTTAGATTAGTTCTTTATGCTTTCAGATAATTTTTTGGGGTTGGTCTAAAAACCTGAGGTCGG
GATGGGTTTCTGATGGCCAAGGGAAAGCCAATGCCGTGAATCTACAATGTGAGCAAGACCATCTGGGCAA
GGCTAGTGTGGCTTTGTCAGGGACTGCACTAAAGCCAGACGGCCCCTTCACTGTCTGTTCTCCCTAGGTG
AGCTATGGTAGGTCTCCTCTGGGCTGGAGGAGGGTGGGACCTGGCTCTCACTGCATCCCTTGTCCTCCTC
TGCAGATCAGCAACGCTATCCTCATCATCTTCGTCAGCTACTTCGGCAGCCGGGTGAACCGCCCACGGAT
GATCGGCATAGGGGGTCTCCTCCTGGCTGCAGGGGCCTTTGTCCTCACCCTCCCACACTTCCTGTCAGAA
CCCTATCAGTACGCGTCGACAACTGCTGGTGAGTGGCTCCGACTCCTGCCTGGAGGAACTGAGGCAGGGT
GAGCACGGTGCAGAGCAGATGGTGGTGCTCCTGGGGTTGGGGGTCTCCGCTCTGAGTCACCTTCTTCCCT
CTCCCGCTAGAGACTGTCCTTGACTTCAGTAGTTCATTTTCTCTGCAGACTACATCTGAAGTGCAGAACC
TATTGAAGCATGCAGAAGCATGAAGTAGCCCAGCTACCTGGTTGTTAAGGATGTGGTTGCTGGCCTTTCT
CTGTGAGTCCTTGTGCTTTGTGCCCAAGGCAATCCAAACTCCACCTGCCTAGAACCAGAACCCACGGAGG
CTTAGAGAAAAAGAGGACCAGAGTGTGGGACATGATGAAGTTTGGGGGGTAGGGGGGGTCCTGTTTGTTG
TACTGTCAATTTCGTTTTAACTAGCATATCGCTTGTATGGTCCTTTTGTACGGTCCACTCGCTGATGCCA
GGATATTAGTGGAGTGTAGAAAAAAGTTATAATATGAGGTTTTATGTAACTTCTCCTGGAAAAATATGAA
CAAATGTCTGTTCACGCCATATAAGACACTACAACAACCCAAATTCTACTCAACTGTAGTTGGTGAATCA
ATAAATTCATTCAGGTTAATCACAGGAATATAGGTAAGAGATGATAGAAATATGGGTAATTCAAAGGCAG
CTACATCTCCAAAAGGCATACCCATCATGGGTGAAGACTCACAAAAGCTGTATGTTTGGAGCCTCCTGGA
AAGGTCTAGGTAACTCTAACGAGAGTCTCCTCTTCCCCAGCAACTGATTCCTGCTTTGTATGACCTAGGA
GGAAGCTTTGTAAATCTTGTAATTTTCGTGAACTTCTGAGCCATACAAGTTTCTATAGCTCCCTTCATCT
TACGAGTTCCCTACTTCCTCTAACCTGAAGAGAAGGTTTCAGTTGGGAAGAAATGGCTGCATCGTAGGTC
TTGACAGTTGAAGGTACTCAGATACTATTTCATGTTCCAAGTATCTAAGAAGAGAGAGAAGATGTGGGGG
AGGTCTTATCTCCTGACCCTCAGATAGGGGGCTTTTTCTGTGACCCAAACTTCTGCATTTTATTGGGGTG
TCTTGTGGCAGAATGAAGTAGACTTTAGAATTTATGTGGTTAAACATGGTATTGATTAGTACTGAAGTAT
CTATGAAAAGGTAACTTTTCTTTAGTCTTAATTTAAAATTATTTAATTAGCATATATTAATTATACATAA
TAGGTTTCACTGGGACATTTCCGTACATGTACATGATGTACTTGATCATATTCACGTTCTTTCTCCCACC
CTTATTAATCCTGTTTCTTTTCTACTGTTTCGTGTGTGTGTGTGTGTGTGTGTGTGTGTGTGTGTGTGTG
TGTGTGTATTTCATTAGGGTTGATTAAAGGAGTGTAAGTGAGGGTTTGTTTATAAGAACATGGGCACCTT
ACCACAGGCTTTTCCACTGAAGAAAACGTTGCTCTTTTCCCTACCAACCCATCAACTGATTGTAAATTGT
TGTTGTTTTAAATTGTTTAGTTTGAACTTTATACAAATTCTAGACAGCTCTTTGTTGATATAAGCTGTAC
AAATATTTCCTCCCCGTCTCTAACTCATCATGATATCCTTTTTAACAGACTATAAAGCCCTTCCAGAACA
GAAAGAGGTTTTAATTTTGACAAAATTCAATTTTTCCTTATTTTTTTCCCTTCCATGACTCATTCTTTCA
GTACTGCACTTAGGACCTCTGTCTGACCCCAAGAGAGAGATTTTTCTATGCACTTTCATGGGGTGTCTCC
ATTTTTTACATTTGCATGTAGACTTACGATCTGAGTTAATCATTTAGTTATAAATGCTCAAGATTTCCCT
CTTGTGTATGAATGTCACTTTTCAGCCCAATTTATTACAAAAACTGTCTTTTCAGCATACCATAATTTTG
TACCATTATAGCAGATAAATTGTCTTGGGCTAAGGCTGCAGGTCAGTGGTAGAAAGCTGACCCTGAGTTC
CACCAACACAGAGAAGAAAGCTGACGGTGTCTGTGGGTTCATCTCTGGACTTCCCTTCCCACCCCATGGA
TCTGGGTGTCTCCTTTGCCAGTACTGCAGGCCCCTCCCTCCCCTCCCCAGTGTTTTATTACATTTCATAT
ATATATATATATATATATATATACTTTGAGATCTAGTTGTTTATATTCAAAAGAAAACATTTTTTTCTGG
AATTTTGATTGGGACTGCATTAAATCCATAATCAACTGGCACACCTGTTGTGTGTAATCCTCTCATTGTT
AAATAGTTTGGCTCACTGCTGCCCTGTGTTCACCACTGACTGATGACTGAACAGGCTCTAAGCTGATGAA
TGCCTTTATCCTCCAGGAGAACATAGTCCCTGGATGCCTCAATGCCCACTGTCTATCACTGCTGCCCAGT
GCTGTGGATACTCCTAGCTTCCCCTCCCCACCCTACTCAGTGCTATTAATATTCACCCACAAGGCTAGAG
GTTTCTGTGTTCCTATTCTTCTGGGGAGGGAGACTTTAACATACTTTGTTGCTACAATGTTCCCCTATTC
CTGAGCTAGATCTTTTGTTGTTACTTTGATGAGTGATAAAATGTCAGAAAAGATCTTCCTTTCGCCCTTG
CTCTCTAATTGGTTTAGCTGCAAACAGCCATGAAATGAGTAGTTATCCCTGCTCAGAACTTTAAGGAAAG
TGCATCTTTGTCTTTTGGCGATTCTTAATGGACCTTCTCCATCTGTCTCATTATCAGCTCTTTATTTCTA
GGCCTCCTGGTTTTAGTCTCTCTAGTTGATCGTTTTCTTCTTTGCTTTGCTATCATAACATGTTTGAATG
TGGGCCAATCTGTATCTGTCTGGGAGTTCAGGTTTGTTGGGGGTTGGGAGTTAGTTGTTGGGATGTATAA
CCAAGATTTTTATGTAATTCTAAAATATGTCAGTCAAAAATGCTTCTGGTTTTCTTCATCCTTCACTCTG
GAACTTTTATGTAGATATACACGGCAGTCACCCTCATCTTGTAACCTCACTTTCAGTGGGTTGTCCCTCT
GTACCATCTTTTTAGTGATTTCCTTGAAGTACGTCTCCCCATTCTGTGAGTCCCTCTCTAGCTTTGTAAA
TCAGAACCAAGCCCTTCCATAATGTAAAATTTAAGAGCTGTCACTGTTTTGCTAATTTCTAGGAGATAGG
ATTTGCTTCCAAATTTTTCAGCCCCAATTTTGTGATTTCTTAATGCTTTTTTACTGACATACATATACAT
AGGTATGTTTCTTTGATGTCTTCAAGTATTAAAATATATTTATGTATGTAGCCTTTTTTGATATTGTTCC
AATAGCTCAAGCTTTAAGAATGTGGCCCTGTCCCCTTCATTTTGTCTGCTTGCTCACGTTCCTTGTGGGT
CCTCCCTTTGTATGTTCTATCATGTACCATGGAGCAAACTGTAATAAGTTATATGTGGACAGATTGGGAG
TGGATCTGGTTTAATATCCGTTTGTGTACCGAGTGTGCACTATCTGCAGGGATCTAGGGCTCTGACCCAG
CTGGAGCAACCAAAGAGCCCCAAGAGTTGCAGTTCTCCGAGAGGGAGTCAAAGAAAGGCAGCTCTTCCAC
TGCGGATGCTGCCCAGAGTTGGTGGCAGGGGCAGGATGCAGTGGGCAGCTAGGAGCCAAGGTTGGGGTGG
GGTTTGGGGGTCACAGGACAGGAGCCAAAGTAAGGCAGCTCTTCCACTGCGGATGCTGCCCAGAGTCGGT
GGCAGGGGCAGGATGCAGTGGGCAGCTAGGAGCCAAGGTCGGGGTGGGGTTTGGGGGTCACGGGACAGGA
AGTGGCTGAGTTAGCCTTGAGCAGCTTCCAGCGACAAAAGCTTCTAGAGAGAAAACTCTTCCCTAGAGTG
TTACAGTTCCATAAGACAGGCACTCTCAGACGATTCGGTGAAATAACTCATGCCCTTTATTCCTTCTGGA
TAGGATCTTATAATACGGTTTTCGGGGGGGGGGTGGGAACTGACAGTGGGTGCTTCCTATTGTCTTGGTC
TGAGATGTTAGGGATGCCTCATCTGCATGTGGAAGGACTTCAGGTGCTGTCCCTGCATGACTGATTGTCA
CAGTCCTCTCCATGGGGTGTCGTGGAAACCCTGTTCCAGGACAGGAACCTGGCCGGGAGTAGATTTAAAC
ACCTACAGTTCTCAAGTATGATAATTGCCAGGGTTTCTGAATCCACTCCACCTTACCAGGTTTTCTGCCA
GGTGGCCCCACAGGGCAGCATCATGCTTTACATCCTCGTGTGGACTTAGTGCTTCAGGACCACCGCTGCT
TTGGAGGTACCTTTGTATCAACCTGTTAGCTCAGGATGATACTGCCCTTGTCTTGAGAGAAGTGGGGGGA
GGTTATGGCTTGGTGATGAGCGGCAGTGAGGGCCCCGCACTGCAAATGTTCATCCTTAGTTCGATGCAAC
AGAGCTGTGTGATGTTGGTTGAGGATGGGTGAGCCACCCAGGAACGTGCTGAGCAAGTAACTGATTCCTG
TTTGCTTGAACATCAGGCTCTTCTAAAAGTAGATGTAAGGGCCAGAGATGGATCATTTAGAATGGCCGAG
GGAGGAAATTCCTCTATGGGTTGTGAGGCCTGTGCTAGAGATGGGTAGTTGGTTTGAATGGGCGTGGGGA
GGATTATAAGCAGGAGTGGGAGGGGTCTACAAATGAAGTGTCTACAAGGTTCTGGTACTGTCCCTCCTTC
CTCTGGAGCGTCCTAGTCATCTGCATAGGCGTGTCGCAGTCGCAGTCCTTGACTGTTTAAAGGCTGAGCC
TTGCTTCAGGAGAGACAAGGACGGAGACAGACCCAAACCTGTGGGTTCTTCCCTGTCCTTTCTCCCCTCT
ATCTGGGGGGGAGGCATGGAAACCCAGCTCTGATGGAGAGTCAGGCCTTGACCTTTTTGATGCATGAAGA
CGGGGCTAATGAGATCAGCAAGGGAGCCTGGAGCACTGGGGCCGAAGGGAGGGCACTTTGCCAGACACCA
CACACCAGGCTGGTCGGCTCCCCGCGGCCTGGGCCCACAGCCCATGGCATTTGCTTTGCACAATGTAGCC
ACGGGAGGAAAATTAGCGATGGCCTCACTCTGCTCCTTATTAGGGGTGGCTATTCAGATTGAGTGGGACT
TTACCACAAAGCCCAGATCACTACCAGCCCAGTCTTAACCCCGATGCTCCTGGCCCAAATCATCTGAGAG
ACTGTGGAAAGGGCTATTAGAAAGAGCACAGCTTGCCTGACAGGACCACAGTGTGCACAGGCTAGGAGAA
GGGGCTGGGCTGTTGTTGCTCCTGTCTGTGCATGTTCTGATTGCAACGAGGGTAGCACTTGCTCTGACAG
ACACTCAAGCAACATCTGGAAGCGTTCAGGGGTAGACTGCCTTCGGGGTCCACCTTTCTAGGGCCAGGCA
ATAAGAGGAGGAGGCCCGTGACCCAGGAGTCACTCTGTGTGCTTTGGCTGGGCTATGTACTAGGTGCCGA
TGCAGTGTGGAAGTATGGTGTTAATATGTTCAGATAAAAATAGTCATTAAGTCTCATTTTGTGGAAGGAA
TTATGATTTGTGGCTCCACTTCCAATATACTCTTATGGGAACTTCAACTTTGAATTATGTGAGGTCATCA
GAGTGGGAGCGACACCCAAATGTGGGTGTGTCTGAACTGTCTTCTGGAGCCAGGCAGTCCTAGGGCTGAA
ACTAACCATTGAGACTCTCTAATGGGAACTACAGCCTTGAGCCATTTCCTCCTTGCTTTGAGTCTTAGCA
AGGTCTAAAGAGAAACACTTCCCCATCATAAGGCTCAGGTGTAGCCCCTCAGAGTCCCCAGTGAGAACAC
ACATTGTGTCCTCTGACAAGTGCTTTGCTGTGGGCTGGGTAGCGTGGGAGGACCTGCAGGTGATCTCTAG
TCTTCATGCCTGGTTCTCCAGCACACCCTCATTTGGGTGTATGGTCCCTCTGGATGAGAACTTGGCCTGC
TGTTCTGACTAGTTCCCAGAGCACGCTGAATGAAGTTTCATCTGGAGCCTCTTAAAGTCACAGTGTAGGA
AAGGCTCGGTCCTCACTCCCATTGGTAGGCAGAGCATGGACTTTGACAGTCCAATCCCATCTCAGCTGCT
CTCTTGCCAACTCTTCTTAGGGGAGGCACTCCTCTTTTTTTCTGAACCTTACCTGTAATACAGGAGCAAC
AATACCTTCCCACTCTGTGTTCCCGGAATAGAAATAGACATGAAGAGCGTGGCTCATGGTGGCTATTGGG
GAAACCTACCTCTATTTGCTTGCCTTGCCGTCGCATTTGAAATCTGTAGTCGATCAGTTTCCGCTCCACT
CCTCTCTTCATGAGGATTTGCTGATTTCTTCCCCCATGGAGCAAAGCGGCCCCAGCAACGACCATGGGAA
ATGAAGGGAAACTATGATCTTCAGAGGGGGAAGAAAACTGAAGGAAGGCTGAATGTTGTGGGAAATCACA
AAGACCTTTACAACTAATTGCTAATTTAAAAAAGTCCATGCAGGCATTTATGGGTGTTTAGATTTTCTAA
ATGTCACTGGGGTCCGGAAATGTGCCTTGGCGCCTTTAGAGGCTAGCTACCCAGCCAAAGCCTCCATCCA
AGCCCATGTCTAGAAGATACAGGGAGGCTTTTGTGGATGGGAAACAAGCCAGCGTGAGACCGGTTCCCAT
AGGAGAGAAGAGAGGGGACTTATGGGAATCAGTGTTTGGAAGGACTAGGTGTAACCTGGTGAAAGATTCA
GTCTAGTCCTTCCGGAAGTCAGCCTGGAAGACAATGGTTGCTGGGGCAGGAAGTCTCTTGAAGGCTAAGC
AGGAAATGAAGGGAAGCGTTGTATTTTGGAAGCAATCAAGAAAAGAAAAGGAAGAAGCCCTTCCTGGTCC
TTGATGTCTGAGGCTGTTGGTCCCTAGGCTGTGCTCTTCGTGATGTCCCCAGGAAGATGGGCAGAGGGAA
AACACACAAAGCGGAGCCTTGGGACCCAGAGTGACTTCAACCCTTCCTTCCTCGGCTCTCGGCCTCCAGA
GCTCAATCCATCTCCTAAGCCTGGAAGGGGCCCAGGCCCGGGGCTCAGACAGAGTCACTCTGGAACACGA
GCTCGGGTCTGTGGTAAACACCCAGCTGTGGCATGAAATGGAGATGCTAGAAGCTCCGGGAAATGAGGGG
CAGGCTCATGGCGTGCAGGTGGCCCACGTGTGCCTGTGCAGGGTATGTGCAGGGCAGAGCTTCTCTACGG
GCCGATGTTGGGTGAGGTAGGGAAGTGGCTGGGCATGCAGAGGAGAGAAAGCTGGTGTGTGGGGGGATGG
GTACTGGTGGCAGGAGTGTGTAGACGCAGGGGTGAAGGTAGGGCGGTAGGAGGGTTGTGAGGAGGTGGGA
GTATCTGGGAAGGAGGGTGAAGAGAACATACATAAGAAGAAGTGTGTGGGTAGGGATGTGGGGGACAAGC
GTGCACGTGATGGGACTCGGGTGGGGTGGCAGTTGGCTGGAGAGCTGAGATAGGGTGTTACTAAAGAGTA
AAGGGAGCATGCGTGTTGGGGTGAGTGGGTGGGTAGTCAGTGGTTGCACAGCCTGGCTAATTGGAACTGT
TTAATCTGGAAAAAAAGGAAGAAGACAAAAAAACAAACAAATACAAGAAAGGAAAATATCAAAAAAAAAA
AAAAAAAACCACATACATGCTATTAACTCAACTAAAAGAAAATCAGTTCCGAACAGATGAGTAGACCAAA
AGCAAATGTGATTTGACGTGGACCTTCTTAAAGTCATGGTATAGGAAAGGAGAGGTGTAAACCTGAGGGA
TGATTGTAGGCACCCAGCTGCTGCACTGAGTGGCAGGGAAGGTCACAACTCTGAGGTTGGTGGGGCAAGC
TTCTCCACTGCAGTGGCATGAGAGACAGCCCCTCTCCAGGCACTAGCTCCTGTCCTAGGCTGTCCATGCT
CTGGGTCTTTGAGCATTGTAGTGGGTTTGGTCTGATGCTGCTTGTATTCTAATGCGGAATACTGGTTCCT
CAAAACGTGGTTGCCCTCCGCCCCACGAGCAGATCCTCATGTGTTCAAGGGATGCCATGTGAACCTTCTC
CCCAGTTTCACTGGTCAATAAAAGGCCTGTGATTGGGCAGGGAAGGGAGAAAGGTGAGACTGGAGGTTCA
AGAGATAGGGTGGCAGGTAGAAGGGGGAAGGAAGAGGAGGGATATAAGGGAGGAAGAAGCTGCCATGGAT
CAGAACCGTGTGGCCAAAAGGAACAGCAAGTAACAAGGGCCCTTATAGCTAGGAAATACGTTAGTATAGA
CCTGCCCAATCTAGGCATATGGCTTATAAATAGAATTTCTGGATTGTGTGTCTTTTATAAGGGCTTATTA
GGATTGAAATTTTAACTACATAGCATCTGTGTTGTGGGCAAGTCAGGTGCATTGAAAGCATCCTCCTCGT
CCTCCAGTAGGAGGCTAAATGTGGCTGGCTCCTCTCTACCTTCTGGGCCCTTGGGAACTACTTACTATGC
AGCCATTTTTCTTGTTCAACAGTGAAGCCAGGTAGTGGGCCTGCTGCAAGTGTCTTTGGAGACAAGCTCC
TTCCTGCCCTCCATTGAGTCACTCTTCCTGGATGGTTGGTAGACTCTGTCTCAGTCCTACCTAGGACGGG
GGAAGGTCTCCCTAAGAGCTGTCATTCAAGGCAGAGAAGAAAGGCTTTGGGCATCCTAGGAAACAGTCAC
TATCCCACTGTAGGATGGGGTTTGAAGAAACCGTTAGCCATCACTATAGGAGGTAGTGGACTCCTGAAGG
CCCAGGGAGTCAGAAAGGCACCTTCTATTCCTCTTCCTGTACTTGGTGAGGCTATTGACTGCCCAGACAC
CTCCTGTGACTGAAATACTTGACATGGATGACCCAGGCACACCTAGGCAGTGGGCACCTTTCTCCCACGA
CCATGCTTGCGCCGTCCATAATCAGAGAAAATGGAAGCTCGGGCATTGAAATAGGGAAAATAACACAACA
GTCAACTGACCCACATCCCAGGAACTAGCTGTTAGCAAAGGCTTGTGGGTATTCTGGAATGTTCCAGATA
TCCCCTTCCTGTCTTCTCCCTAGCCTGGGGTAGAAGAAGTGAGGAGAAGGCTGAGTAGGAAAACTCCCTC
CTCCACCACACACCCATCCTTCAAGAGCCCTGGTTATGTCTGGGTTGTATTCCATATCTCCAGAAGCTTT
GGTTATAGAAAGCTTTAGTGGCTGAAGTGCTTGAAAACCATATCAGGCCATGGGCAGACATTTTTATTCA
AGTACCAGGTAGCAAATATTTCCAGCTTGCCAACCCCAGAGACTATGAACCTGTTTAGCACTGTCCCAGG
AACACCAGCTTCCACAGGAGTAGAGACCAAAGTGTCTAGGCGTGTGCTGTAAAACTCACGGGCACAAACA
GGGCAGGGATCCATCCGATCCTAGCCTCATTAGTAGTTTCCCAAGCCCTGTGCTACATGGTTCTCCTGGC
CTAGGCTTACCCTATCCCTTAGAGCTGAAACCAAGGCTATGAATCCACCAGGCTGCCTTGATGTGCCCAG
TCACTCTCTTTCTTAGATATGGTACTATCTCCCATGGGTTCTAGTCCCTCCTAGAATCATCCAGAAAAAG
TCTGTTAGTCCCAGCTACAGGAATAGCCACCCCATAGCTGGGCTGAGCCTGCTCTTCCACTGCGGGCTCT
TCACTGGACCTCAGATAATAAGGTTGCAGGGCTTCGGGGTCTCCTCAGGAAAAAGACAGAAGTTTCCGCT
CTGGATCCGAGGATCCTAAAGCAGGGCTTAAGATGTGGATGTGTGATCAAAATAGCCAGAAACTCAACCC
TGAGGATGGTTTTGTTGTTGTTTGGTTTGGTTTGAGACAGGGTTTCTCTGTGTAGCCCTGGCTGTCTTGG
AACTTGCTCTGTAGATGGGACTGGCCTTGAACTCAGAGATCTACCTACCTCTCTCTGCCTCCCGAATGCC
AGGCCTTGCCTTGCCTATTTACTGTCTAGCTGAAGCCAAGCCTTGTATTTGATCCCTGTAAGAGTCACTC
TCCTTGGTTGGAACCCTGCTTTTTTTCCCTTGGGTTTAATTTTTTTTTTTTTTACTTTTCGGCCTTCGGG
ATGTTTTATCGGCCATTCTCCTGACTCTGTTGTTCTTAGGCCTGCCAGCCATGTCCCCAGGACAGAGCAT
GAAGAGTGACAGACACATAGCCCTTCTACCTCAGGTATACAGTAAGCTGGTACCCCTGAAGATGCTCCAG
ATATGGGTCCCCATCTTACCTAGCAGATATAGTGACAGACTTTGTCAACAGGCTGCCTCCAGTCAAGTCA
TGCCACAGCTTCTGCTGGCTAAAGAGCCTCACTAACAAAGAAAATGTGTGTGGGGGAGGAGGCATGGATG
ACTTTTGATGAACTGATGTTGGCTCTGCTAACTACTCTGGGTTTTCTTCAAAGAGCTCCCCGGCAGCCTG
GGGAATAAATCTGTGAGCAATAACTTCTATTTCACAACAATGAAAGGTGCTAGTGAAGTGCCTATCTCCT
TAAATATTAAAGTCGGTGCTGCTAGCTGTCATCATTTTTCTCCCAACCACTAAAGAAATGAGTCTGATGC
TCAGGGTGCCTGTGCACTGGCAAAGGCTATAAGGAACTAGGAGAGGGGAGTCTCAGGCCAGCCATATGGC
TTGGGATTTTAAGTCTTGCATACAGCAAGCCTGCTCCCTTAGGCCCAGTACAAGTAGTATTATTTTAGAA
CCCTCCTATGACAAGGGAGGCAGTGTGGGAAGAGAGTTTCAGTCTCAGTGCACACATACATGTGTGCACA
TACACAGACGTGCCCAACACTAGCACACTCCTTTCCTGTACATGTACGAACTTTTCCAAGAACACACTTT
TGCCCTGTGAGTTCTTTGTTGTAATCTGCTATTGCTGGAAACCGCCCTCAGTCTTGCCTGTCACTACTGG
AGCCTGTCCCTGAGGAACATGGCCTTATCCATTTGAGTTTCTCCAAGTGATTCATCATTTTGAATGAAAG
TAGTTTTATTGGAGGAACTTTGGGTCCTCGATTATGATATACTAATTATTTCTTATAAAATAATGCTTTA
TAAAACAAAGTTTTATGAATAAGGAGAGCACAAAGTTGTGAAGGAGTGGGAAGGTGGGGATGGACGTGGG
AAGAGCTGGGGGGATGGGAGGAGAGTGACTATAATAAAAAATACATTGTATAAAGTTCCCATAGGATCGT
AAAATAGTATATTAACTGTTTTTCATTGCAATGACACATAGTGATCACATAAGCATAGCATGTGACATTT
CAACCTATGTACACATGTGTGCTGATCTGATTGGAATGGCTGACATACCTTCTATCATGCTAGCCACTTA
CCATTTCTTTATGTTGGGAACATCATCCCTGCTTCAGCCCTGTCTCATGCCTGCCTGAGTCCATGGGTCA
CATCTCAGGAAAGTTGACTTCTTGACACAAAATCTCCTATATTGCAAAACCACAGAGAGATCCAGAAGTC
AGGACTGGAATTTTGCAGATATAAAACTCAGCGTGAGTCAGGAGGTTGACTCGTGTGCTGCCAGGGTGGG
GCTGAGTAACCTCAGGAGGGGTGAAGCCAGCCTGTTCCTAGTTAGCACTGCCCAGTCCTGTAAAGACTAA
TGGAACCGGACCCCTTGTGTCCTACTGGGGTTAAAGAATGTGCTTTGATGAACTTGTGCCTTCCAGGAGT
CTGCCTTGGAGGAGATAGGAAATAGACTAAGCTCCTGTTAATAAAAAAAGTTTCTTTTCCTCCTAAGGAG
CCCTGCAGTATAGAGGCAGGCAGCCTTGGGTGAGCAAATGGATTTCAGAGAAGTTCTGTAGTGGCTGCAA
AGGGTGCCACTTATCTGTCTTATTTCAGGACTTTTATTATTTTTAAAACGTTGTGTGCGGGGGGGGGGGG
GGGGGAAGGGGACTGGAGATATAGAGATGCACACCTTTGATCCCAGCACTCCGGAGACGGATCTCTGTGA
GTCTGAGGTCAGCCTGGTCCACAGAGTGAGTTCTAGGACAGCCAGAGCTACAGAGTGAGTCCCTATCTCA
AACAAACAAACAAATACACCAACAAAAATGTAGGACTGGAGAGATGGCTCAGTGATTAAGAGGACTTACT
CTTGCAAGGATCCAGGTTTGGTTCCCAAAGCCCACATGGTGGCTCATAACCATCCATAACTCCAGTTTCA
GGGAAGTCAATGTCGTCTTCTGATCCCTGAGGATACCGCGTGCACATACATGCAGGCAGAACACTCATAC
ATATGAGATAAATAAATCTATAAAAAAATTTAATTTGTATGTGAGGTAACGTGTATGAATGTGTTTGGAT
GTATGTTCATATGTGCACATATGTCCATGCACACATTTATGTGGAGGCTAGAAGTCAATGTTAGATGATT
CTCAGTCATTCTTAGCTGTATTTCTTTGAGACAGCATCTCCCACTGAACCTGGCGCTCGCCTGTTTATCT
AGGCTGTAGGCCGTGAGCGCCAGGAATTGGCCCTATCTCTGCCTCCCCAGTGCTGGGATTACAGGTGCAC
GCTGCCACGCCGGAGCTTCTCTCTGTGTGCTGAGGAGGATCTGAAGTCAGGTCCCCATGTTTGCTCGGCC
ATCACTTCACTAAGTGAGCCATGTCCCCAGCCACAGTGAGCAGCCGGGAAAGCGCTCAGACTTGAGAAAT
GAGATACTTGTTGATCCCTTACACGGGCTCAAGCTCTTGGAAAAAGCTAAGAACTTTGCCCCTAGGGAAG
AAATGGGAGACTGCAGCCCAGAAAGCCAATCTGTCCTTCTGTCTGTTGAGAACCTGCCTGCAAGGATGGT
CCCCTCCAAGGCCTGTTCTTCCCATCTTCCTGCCTCTGCAGTCACATATCTATGATGCCCACCTCATTCA
TGCTGATTATTTTTGAAAAGAGCTTAACGGTCTTTAAAAAAAAAAAAAAACTAAGACCCTATTTTCACCC
AGGGTGTGGTGACCTAGACACCCACTAAAGGGGGTCCATGCCTTGTTCCTAAGCTCCTGCATAAGGCTCC
AACTGCTGTCAACCGCCCTGGAGGGCAGGGAGCAGCAGACCTCCTCAGAACATCGTGCCCACTGCTCTTC
TCTGTTCCTCGGTGCTCCAGGGTCAGTGCTCACCACCTCCAAGGTAGCCGACTGCTGTTCCTCTGAGATG
GCACCCATAGGGAAAGAAGCCCTGCCCTAAGGGCTTTGTTTAACTACTTTTGCTCATACATGTGTGTGTG
CATGTACATGTATGCATTCATACATGCATGTGGGGGGGTGGGGGGGAATACATCCGGAGGCCAGAGGTCA
TCCTGGGTGTTGTTGCTCGCTCAGGAGGGTCATCCACCATATTTTTCGAGACATATTCTCTCACTGGGAC
TTGGGCTGGCCAATGAGTCTAGGCTGGGTGGCCACATGCCATGTGATCCTCACCTTTCCAACACTGGCGT
TACAAGCGTGTGCCCTCATGCTGGGCTTTTTATATTGGTTCCAAAGATCATATTCAGATGTCATGCTTAT
GAGGGCAAGCACTTTATGAACTCAGCCATCTTCTCAGCTCCAACTACTGTCCCTTTGCACCAGTGTCACA
GCTGGATCTGAAGTAGGATTGGGCCCCCATTCAGGACTGGCATCGGGAAGGGCATGTTCTGCAGGGAGAT
CTTGAATCAATGTGGGAAACAGAATAAAGCCATGGAGAATTTGGAGACTTGGATGTAGTTGGGCCAACTG
ATTTTTTTATGCATATTTCGAGTAAGATGTATTTCTGACCTGCATATTGACACTTTTAAACTTAATAGTA
GTGAAACGATCCCCAGAGACTTTGGTGGCTGAACACTTCGCTGGATGAAACTTCAGGAGGATTTCTGTGG
CTTTGGCATACCACGCAATATGGCACATGAAGACTTAATAAGGCAGTCAATGTTTTTACGATCTTTGATG
ACCATTAAATCTCTCTCTCTCTCTCTCTCTCTCTCTCTCTCTCTCTCTCTCTCTCTCTCTCTCTCACACA
CACACACACACACACACACACACACACACACAGAGCCTTTGACAGCCAAAGCAGTAACGATGGTTCTCAT
GAATGTCTGTTTCTGTCAAATAATGAGGACGGATGAAATCTGGAATTAGAGTAAACTTAGAACAAACTCT
ATTCTCTGGGACTTTTATTTCCCCGGAGAGTCCCTCAAGTCGTCATCCCCTGCCCCCTGCTCCAGTTCAC
TCCATTCTCCCACCACAGTCCTACCTATTCATACTCATACGACCATGGAGCCCCCGCAAGCCCTGACCCT
GAGCACACAGCGACAGCCTCACCTCTTAGAGCTCTAGGCAGTTCTGGCTGAGGATCTTCCTGCCCTTGAA
CTAGGGCCTTAGTCTACCCAGAAGCTGTATTACAGTGCCCTGTCAGGGTCCTCACCTGTCTTGGGGTGTC
CTTGGGGTACTTCCTAAGGCAATCTACCCACCTGCAGTAGCTAGATTGCATACCCCCCTCCCAAACTTCC
CTGTGAACTGTGTCCCTCACTTGTGAGTGCTGCGACACCCTGGCTTCCCATTGTGCCCTCTTCTTGGTTC
AGAGGCCCCTGGTCTCCAGAGAACTTTTTCCAACACCTCTCCATCCCCCTGCCATGCCAACTCTGAGTTT
CCCATTTAACCTTTACTCTCTGTCCTTTGTGTGCTTGGCACTATTTTTTTTTTCCAGTTACGGTGAAATG
TGTAACGTGAAGTTTGCCCTTCTACCACTGTAAGATGCGTGCTGGTGGGGCTAAGTCAAAGCCACCCGCC
TCCAGGACATTCTTATGTGAATCGATCCTATGCAATGTCTTCATTCGTTTCTCCCTTAGCCCTTGACAAA
CTCCCTGTCTTTATGATTTCAGCCATTTTAGAAGCCTCCCATTAAGGGAGTCAGGCAGGACTTGGCATTT
TGTTTGGCTTCTTTCACTTAGCACAGTGTCCCCAAGGCTCATATATGTTACCATACAGCAGAATTTGCTT
CCTCTTTAAATTGAAAAAAGATCCTATTGTATGTACCATGTTTGACTTAATTACCAGGTTGTTTCTATTT
CTTGGTTTTCACCAACAATGGTGCCATAAATATGGGAACATTAAGATCTCTTCAGGGCTGAAGAGATTGC
TCAATGGTCAAGAGCACTGACTGCTCTGCCAGAGGTCCTGAGTTCACGTCCCAGAAACTACACAGTGGCT
CACAACCATCTGTAATGGGATCCGATGCCCTCTTCTGCTGTGTCTGAAGACAGCTACAGTGTACTTATAC
ACATAAAATAAATAAATCTTTTTTTAAAGCGGGGAGGTGGATCTAAAAGAGGAAGAAAAAGAAAAATCCA
GCAAGGGCAGTGGCCGTGCACACCTTTGATCCCAGTAGTTGGGAGGCAGAGGCAGGCAGAGGCAGGAGGA
TCTCTGTGAGTCAGGACAGCCAGGGCTATACAAAGAAACCGTCTTGAAAAACAAAAGCCTCTTCAAGCCT
CAGCTTCATCTCGATTATTACGGGACTCATTGCAGAGTGGCTGCAGCCTTTCCAACGCTTCGACAGCAGT
GCCCAGGGGTCTCTCCGTCTCAGGATCCTCACCAACACTCGCTGTTTTCCGGCTTTTTGATGCTTGCCAT
CCTGTGGGACATTACATGCTATCTCATCGCTTCCTGTTTCTGTTGGCAAAGCAATACTGTAGTTGCTTTT
TTCTTTTTTTTAAATAACCCAGGGGCCCTTGTGGGTTGGAGACTAGGTAAGGGCCATGCCTTTGTCCCTC
TGTCACCTGCATGAGGAAAAGGCCTTCTCTCACAGTCACTAGCCAGATGGCTGGGAGACTAAAGAGGATT
TGAATCTTGGTGTGACTGTTTTTCAAGAGGCAGGCTTCACCCACACTGCTAGTCAGAGGATGGTGTCTCA
GATAGAGGCACATGCCTTCCGTGACCAAGCAGGCACAGGGACAACTCTTTCTTGGCATTATCATTTGATT
TCAGATGGCCTTTAAAATATTCTTGAATAGATAATGCACACCACAGGACACTCACTAAAGGCGACCAAGG
CCCCTGTCTCCGTTAAAGTCATCTGCATTCTGTGCCACCAATGAGCCTTGGCTAGTGGTTGTAGTAACAG
TTTGGTTTGTTGAGAGGGGTACATGTTATGTGGACCAGGCTGTCCTCTAACTGGTAATCCTTCTGCCTCA
GCTCCTTGAGGGCTGAGATCCCTAGTGAGCCCGCCATGCATGGCTTCCCAGAGTACTTGATAGTATCACA
CCTGGCCTTCGTCCCTCTGTTTTATCTGAGTCTCTCAGTGGCCATTAAAGGAGCCCTTTTGCTGGAAGTT
TGCACATGGGTCTAGTCTAGACCCTGGCATTGAGAACAGGAAGTGATGGGATTCGGCCATAGGAGGCCTG
TGTGGACGGGGAATGGGGCATCAGGGAAGGCGGAGCCCCTGCTCCTGCCCTACCAACTATTCCATGGCCC
CAACCTTTTCTCTCCAGGGGGCTCAGGGGCCCTCTCACTCAGGGCCACATCTCTAGGAAGATCCTAAGTA
GGAATTAAACTGATTCCATTCGTGTTAATGAAAACCCCACAGAGGACGGCTGGTGACGTCAGAGGCTTCC
ATTGGCCAGAATGAAGCAGGAATAAAAGGAATCTGGAAAAGATTCCCTCAAACCCCTCTAGGCCTTGGAA
AGCTGACAACGCTGATTGTTTGGTGTGGGGCAAATTGAAAACAGAGGCACAGAAGCAGCTGCCAAGCCTG
GCAAAGCCTACAGCCGCAGGAACCACAGTGTCCAAAGGCCATACTGGGATGACTGGAGCCCTCCCAGAAG
GGCCTCCAGGGGCAGCCTCGCTCCCCAATCTGTAATATGACAGGGTCTCCAAATCCCCTTGTTTTCAGAG
TCTGCCGTTCTTCTCTTGGTCCCGGCCAAAAGGCCCAAGAAGCATTAAGAGTCTATGATTCTGGGGGGCT
GCCCAGGTGTGGTCAGGTCCCTAAATACCTTTGGCAGCCAGTCCTTCGCCATGGTGTGGTGCTGGGAGCA
TTCAGAAGTGAAGGATCAGGGGTCAGGATGCAGGGAAGGCCAGAGCCAAGCCCAGCGGTGACATAGTCCT
TTGCTGAGGAGAACTCTGGGTTTAGCCTGTGGATCTCCTCTTCCAGATGGGTGGCGCTTTCCTCCAGGAC
TGAAATACGGATCCACTATGCTCAACCCTCTGCCAGCACCAGAACAGAGGGAGAAATGGTTGTGCCTTCT
CCTTCCTGGGCAGAGCTGCTCCTGGTGCAAGAACCGCCATCCTCCTTGCTCTCGCCTCAGTCTCTGTAGC
AAGCAAGTCTTCGGCCAGGCAATCAGAGGACTACTCCTCTAGGACCATGTCCCATTTTCCTAACCCTGGC
CCTCTTTCCCTTCCTCTGGGATATGGAAGAAAGGCTAGAACAAGAGCGGGATAGAAGGACAGATGGTGGA
GGCGGGCAGCCAGGCTGATGGTGACTACTGGGCATGCTCATTGCAGGAAACAGCAGCCACTTTCAGACCG
ACCTCTGTCAGAAGCATTTGCCAGGCCTGCTCCCCAGTAAGTGCCACAGCACCGTGCCCGATACCCAGAA
GGAGACCAGCAGCATGTGGAGCCTGATGGTGGTCGCTCAGCTGCTGGCCGGCGTTGGGACGGTGCCCATT
CAGCCATTTGGGATCTCCTATGTGGACGACTTTGCAGAGCCTACCAACTCGCCTCTGTATATCTGTGAGT
CTTAAGCTTCAGCCGGGCTGCGAGGGGTGGGGATCAGGACCTGGTGCAGCACCTGCTAAAGCTCTTGGGG
ATGAAGTGGGGTCTGATTCACTTGGCCCACAACATGGTGGATGACAAAGACCCATTGTTAATGCCAGTTC
TGCTTCCTTCCACTTGGCTGGACCTCAAGTGGCTAACCCAAGTCCCCAGGCTTTTACTTAGAGGACGGCA
CTAAGATTTTTTGTGTGAGCCTTGTGTTTTGAGGTTAGGTGAGCTGATGTAGATCACCATTTAGCCCTGT
TTTGAGCCTTTGACATCACAGCATGATATTGGATTCTATCAAGTTCACACTAGAAAACCATGCAATTGAG
GTTTTTTTAAAAAATGCAACAACAACAACAACAACAACAGCATAATGCTTTAAGTTTAAATTTGGTATTG
GACCACGTTCACAACTGTCCTGGAGACCATGTGGCCTATGGGCCAGGTGTTAGACACTCTGTAGATCCTC
AACAAATTATAGGCTCTTCTAGGTGATATACCAATACTCCGAGTTGTGGACAGGTTAGCATGCATGAAGA
AGGTAGCAGATGGCCAACTGGTACCTAATCCCTTCCCTTAGAGAGAAAGCCACATCGAGCTTTCTCAGGG
TCACAGCCTGTACCTTTGACTCCACGTGCACCCGTCAGAGCCACCAGCACCGTGCACACACAACACTGTA
ACTATAACACAGAATCCGCGTGATGCTGTACCCATCCTGGATTTAAACTAATGGAGAGTAAATAAATAAA
AGTAGATTTGTGCTCTTGTAAAAAAAAAAAAAAAAAAAGATACCATTGAGGAAAGTTCATTCCAGGCCAG
CAGATCTCTCTGGCAGGTGCTGGAGGAGCCGCAGGCTTGGTTCTGCAAGCCTTGGCATCCGGCCCTTCCT
TATCTGCAGGAGCTGTAATAGGCTTGGGCTTCAGGAGGAGTTACAGGCATGGGCTTGTTGGTCCCAGGGG
ACAGCGAGTGGCACCTTAGGAATCTCCTTCAGTTTGGGAGACTAGGGATGCTGAACTCCCCCCAATGCCC
CTCACTCGGCTTTTTATCCCTTGCTTTAATTTCCAGCCATCTTATTTGCTATCGCCGTGTTTGGGCCGGC
TTTCGGGTACCTGCTGGGCTCAGTCATGCTGAGGATCTTCGTGGACTACGGCAGAGTGGACACGGGTGCG
TATGCAGAGGACACTCATTCTTACCTATCACCCCTTTACTGATGCTTCTGGTTTAGCTCCTCCCTTGTGG
CTGCCCATAGAGAGCAGCGATGGAGCTCTCAGCCCATGTGGGGCCCTGCCCTGAGGTGCCTGTCACTGAG
TGACTGGACACTATTTTGCTACTTTCTTCCTCCTTCTGTGTCCATGTCTGATGTCAAGGAACACAGAGGG
GGCTGGAGTGCAGTGGGACAGATTTTCAGGGGATCATTGCTGTTTGTAGAACAGGCAACCCACTTTGTTG
ATGGCGATGTCCTCCGGCTCCTAAGAATTATGGGACCTCCTTTCTTAGGGCTCCTCAAAAGTAAAGTGTA
GCTGGGCGTGGCAGCGCTCGCCTTTAATCCCAGCACTCGGGAAGCAGAGGCAGGCAGATTTTTGAGTTCG
AGGCCAGCTTGGTCTACAAAGTGAGTTCCAGGACAGCTAGGGCTCTCCAGAGAAACCTGTCTCAAACAAA
CAAAACAACCAAACAACAACAGTAAAGTGTTCTCAAGCTTAGTGGTTACGAGTGCAGGTGAGGATGGCAG
GCACACAGGTTTGGAATTGACTAATGGACAAGAGATTTGGTCTCCTGATCAGTTAGAGGAAGTCAGATGT
GTGTAAGTCTTCCGTTACTGGGATGCTAGAAGAGCCTGTAGAATGGTGGCGGCAAAGTGTGTAGTAGTGT
ATGAACCATGTCAGCCCTCTGGAGACCAGATCATTTCACCAATTATTCCTGCCAAGAGCTAGAGAGGAAG
CACAGCAGTGGGAGGGTTAGCATTTGCCTTCTGTGCAGAGGCTGGGGTGCTCTGGGAAGACCAGAAAGGC
CTGGAGAGGGAGAGCTGTCCCACCACTGTGGGAAGAAAGTCAGTAAAACACCCCCCTGAACCAGAGTTCC
CTTTCGAGTCAGGTCAGAGAGATCGGGCATTCAGCCAACAGACACTTCTATCCCTCGGTTTCTTCTCAGC
TACAGTGAACCTGAGCCCAGGTGACCCTCGGTGGATCGGAGCCTGGTGGCTGGGCCTGCTCATCTCCTCA
GGCTTCTTGATCGTCACCTCTTTGCCCTTTTTCTTCTTTCCCCGAGCGATGTCCAGAGGAGCAGAGGTAA
GTGTCCCAGGGCCCAGGACTCCATGGATGTAGCCACGTAAGCAATGGACACGGGCTTGGAAAAGAAACAG
CTGGGAAGCTGCAGTGGCCTTGGCAAACTGGGGGAAATGAAAAGAGGTCTTAGGACTTAGGGTTCATCAG
AGGCTGAGAAGAGGTACCCCTCAGCACCTTCTTATCAGGCTGTCATCTCGAAGACCGGAGGGTTGCAAAG
GAACATAGTTTGGAAATCCCTGAGTTCATCTAAGCTGGGCTAAAGAGTTAAGAAGCAATTCACAGTGCTC
AATGGGATGTAGAGACAGCGAGACAACTCAATCCTTAGAAAGCCGGCCATGCAAACGTGAGGCCCTGAGC
TTAAGCCACACACCGCCTCTTTTTTTTAAAAAAAAAAAAAAGCCAAGCGTGGTGGTATATAATTGCAATC
CCAGAGCTGGGGGGGGGGGGGGTCAGATAAATGGAAGTATTCCCTAAGGCTTGATAGCCAGACAGCCTAG
GTGGGGAGTTCCAGGCTACTTAAAGACCTCTTTAAAAATCAATATGGGGGCTGGAGAGATGGCTCAGCGG
TTAAGAGCACTGACTGCTCTTCCAGAGGTCCTGAGTTCAACCACAGCAACCACATGGTGGCTCACAACCA
TCTGTAATGGGATCTGATGCCCTCTTCTGGTGCGTCTGAAGACAGCTATAACTATATATTCTTTAAAAAA
AAAAAATCAAGATGTGTGGTGCCAGAAGGATGACACCCAAGGAGGTTGGCCCATGACCTCCACGTGGACG
CACACATGCATGTACACATGTGGACACACACACACACACAAAAGCTGAGCAGGTAGTGAGGCACAGTTAA
CTTGGACATTTAGTGGAGTTTACTAAGGTTTGAGCCTGAATGCCTGGTGAGAAGGGGGCGTCCACTCAAG
GTGTAAGTGAGGGTGTGGGGCAAGGATGGGCTGAGAAGGAGCGTGTCTGGGACAGGGTTGTATCTGCTAG
GGAAAGGAACCTGAAAGATGAGACTAGGAAGCAGCAGAGAGGAGGTTGAGCGGAGCCTTTATGTTACACT
AAAGGGTCTCACGTTTTCTCCTGTGGTTTCTGGGCACTGCAGTGACTGTGTGGTCCCATAGTGACAATAT
GGCAGTTGGCTGAGATTGGGGCTCATGAAAGAATGAAGATTGGAGACCTAAGAGGCTCAAGAGAAAGAAG
GAAAACACAGTTGAGGGTCAGGGCAGTGGCCATCAGAGAGATGGGACAAAGTCCTGCCTGGAGCGGTAGT
GCACAGACTTGGCTGTGGATAGGCGTTGGAGCTGCTGTTTTGTGTCTATGACTTAAGACAGAAAGGGCCC
CACTGTGGGAAATGGAGCTGTGAGCAGAGGTCCAAAGCTTATCCCGGGGAAGCATTTAAGAGGTTATGGG
TGTATTTTGATAGGCTAAGAGTTGGACAGCTCTGAGCTTGAAGACTGTTCCACTCGTAAACTTGTTGAAT
GATGCTAGGCAAGTTGCTTTGTTTTTAAGCTTCCTTTCTTGTAAAGTTCTTGGGAGTAATTAAAGAATCT
GTCTGGGAAGGAAGTTGTGAGGGAAGAGGCGATGGATGGAGAGTAATGGGCATGTTGCCCAGAGCAGGAG
GGCTCAGTTAATGGGAGCATCAAAAAATGCTTATGGTAAAGCCCTGGCCTCTGTCTATGCAGTGGCTTGG
AGAGAGCCGAGGGCTCTGACTGGAGCTGCTAGCTTCAGAGACTGGCGAGGGCACTGAGGTAATGTCCCCA
GAATGATTAGAACAGAACCTAGGGTAGGGGTTTCTTCTGGACTGAGTAGAAAGCAGGGTCCCAGAGAGGA
CACAGAAAGGGGGAGCTGAGAACTGCAGACATCGTAGATCCCTGTGTCCTAGAAGCTAGCCATCTCCAGG
ATCTGCATATGGGACACAGTGTAGTCATAACGCAGACAAGGGTAGGGTCCAGGATGATGCTTTGGGCATC
AGTCATGGGAAAGTCAGATGAAATAATATGCCCATAGGTATAGTGGGAGGCATTGGCACAGATTCACCCT
GGCCTCCAGCCAAGGCACCAGGCTCCTGAAAAACACAGGTGGTATCTGCTTCAAGGGAGCATCCTGCAGA
TGCCCTCCCCCACACACGTGTGGTCCTCACGCCGCTGAGTCAAGGGGCTCAAGGATGGGCCATTAACATC
ACCATGTTTGCTACCTTCAGAGGTCTGTTATCGCAGAGGAGACCATGAAGATGGAGGAAGACAAGTCAAG
AGGCTCCCTGATGGATTTCATTAAACGTAACTGAGCGCCCCAGCAGCCTCGGGTGGGCTGGTTCTGGGAG
GCAGCAAGCCAGGTACAGGGTAAAGGGCAACAAGAGACAATAAGTAAGGCCCGTGACTGCCTCACCTCCC
TTCAGTTCGGAGGGATCCTGCCAAGGACCCTGGTGACCGGTTCTTTACAGGAACCAGTAAATCCCACTTC
AGGCAGAGCCAGTCACCCCCTCTCAAGTCTCCCCTTCTGCTCTCTCTCTCTCTGTCTTCCTATAGTGGTG
GAAGTTGTGGGAGGAGACCTTCCCCAGGGTGAGCAGTGTTTCTATTTACCCACCACCAGCATCCTCTTAG
TCAGACCACAAGCGTGGTGAGCAGGAATCCAAGGACCCTGGGCTGACTTCTGGGAGTCTGGGTGTCTCTG
ATTCTGCCCCGAGGAAGAGAGGAAGCTGTCCTTGTGGATGAGTCAGGGTGACGGCAAGGCTCTAGGAAGC
CTGTGATGCTCTTGCCAAGGCCTGCCTCCTGACAGCTGTTTGCCCGCAGGGTTCCCCCGCATCTTCCTGA
GGCTGCTGATGAACCCACTCTTCATGCTGGTGGTCCTGAGCCAGTGCACCTTCTCCTCAGTCATTGCTGG
CCTCTCCACGTTCCTCAACAAGTTCCTGGAGAAGCAGTACGATGCCTCGGCAGCCTATGCCAACTTACTC
ATTGGTGTGTCAGAGGGCACTGCAGGGCCTGGGTGGTGCCCTAGGGAGGAACTGGGTACTGTACAGTCCA
TCCAGAGATAACTGAGAGCCAGACCGGATGCAGGCAGGAGACAGGAGGGCTTGCCGGAGAGAAAGAATTT
TAAGAGCATTTTTCCTGGTTGGCGATCTAGAGTGATTTTGGCAGGATTCTGAGACTCCTATTCTATTTAG
GAGTGGGTGAAGACTAGGGGCTGCTTTCCTTCCCATCAAGAGGGTAGGAATCTGATATCCTCCCAACTGA
CCTTATCAGGGGTAACAGAGGAGAGAGGGAACATGGAGCTTGAGATATACATAACACCTCTTAGGAGTTG
CCTTCTCCAGGAACTTGAGTCTTGGCCCTGTGACACGTCAGCCTCCTTTACACGATGGCAGTGCAGATGC
TTAGCCCCCACCCACCCAGACTAATGAACCCAGATTCTGTGGCAGGGGTGCTCGTACTTCTTGCAGTAGA
TGCTAAGAACCACTAATGGGGGTTCTACCAAGTGCCTAAATGAATAACTGGGCCCCATCTATAGGTGAAT
TTGACCAAAGATTTGACAAGATAATCAGGGCATGAGATCTGGGGTGACTTCCCTGTTTGCGCTGAAGTCC
TGTGTAGCCTTTAGGGATGCTATCATTTCTCCAGCCTTCAGTGTCGGATCAGGGCATCGCCACCCAAGAA
CGATCTACTTCCTTATTGTTGTCATTGTCTTCTTGTTTTCAATTTGTTTCTTCGAAACAAGTCCTAGAAT
AGCCCAGGCTGACTCTGAACTTGCTAGGTAGCTGAGGATCACCTGGAATTCCTGATCCCCCTGTCTCCAC
TTCCCAAGCGCTGGGTTATAGGTGTCAGAAGTTGACTGCCTCTTTGCTTTTTGATCAGGCTAGCCAACCC
TCTACTAAAGTAAATTCTCGGGATGTACCCAGCAATCACCCAACAGTTAGTGATATTCGGTAGTCACCCA
TCGTTTCTTTACGAAGCTGTGCTCCTTCCCTGTGGTTCAGCTCAAGGCTCCCAGGCTGTGCATCCAGCCC
CTCTCCTTCTCACCTAGGTGCTGTGAACCTTCCAGCTGCGGCCTTGGGGATGCTGTTTGGAGGAATCCTC
ATGAAGCGTTTTGTTTTCCCTCTGCAAACCATCCCCCGAGTAGCTGCCACCATCATGACCATCTCCATAA
TCCTATGTGCCCCTCTCTTCTTTATGGGATGTTCCACACCCGCCGTGGCTGAGGTCTACCCTCCCAGGTA
ATGGACAGCCGGAATCCAGCCACTGTATGTCTCTCTTCTCTGCCTTTTACCTCCTCCGTCCTGCTCATTT
GATCTGATGTCTCACATGGTGACACAGGAAGCCAGGTCTCAAATAGTGTGTTCTATTATGAGATGAGATT
AGCCAGCCAGTGGGGTTGGGCTTAGGGTTTGGTTTTGTAAGAAGCCACAGAAGCTAGCCGGCTTAGTGTC
TGGATGAGCCTAAGCCATAGAAGCCAGGAAATAAGCAATGGTTAATGCCAGCTCTGAACCAGCTAGAGCT
AGAGTCTCACTGAGATGTCGAAGAAAGAGGAGAAGAGACTTGAAAAGGTTTAGAGATGAACAATTAAAAT
GGCGACACACTGTAAAAAACAAGGGCTGCAGAGGCAGGGGCAGGTCAAAGGACCAGTCTGCTTGCTTAAA
GCAGCGTGAGACGTAAGCGTAGGGCAAGCTTTATCTTCAGAAAGAGTGTTACAACCCCATGAGCAAGGAA
CATGCCACTCCCCACCTCCTCCCCCCTCCCCCACAAACCCACCCCAGGTAACCACCTTGAAACGTTTTCT
TTTTTTGAAAGGAAAACATATTTTCTTTATTTATATTTCAAATGTTATCCCCTTTCCTAGTTTCACCCCC
TCCTGGAAACCCCTATCCCGTCCCCCCTCCCCTTGTTTCTGTGAGGGTGTTCATAAGGGAAATTGGTCTG
AAGTTCTCTTTCTTTGTTTGGTCTTCATGTGGTTTAGGTATAAGAGTAATTGTGGCTTCACAGAATGAAT
TGGGTAGTGTTCTTTCTGTTTCTATTTTGTGGAATAGTTTGAAGAGTATTGGTCTTGGGTCTTCTTTGAC
AGTCTGATAGCATTCTCCACTAAATCTATCTGGTCCTGGGCTATTTTGGTTGGGAGACTATTAATGACTG
CTATTTCTTTAGGGGTTATGGGACTGTTTAGATGGTTTATCTGATCCTGTTTTAACTTTGGCACCTAGAA
AATTGTCCATTTCATCCAGATTTTCCAATTTTGTTGAATATAAGCTTTTGTAGTAGGATCTGATGATTTT
TTTGATTTCCATGTCTCCTTTTTCATTTCTGATTTTATTAATTTGGATACTGTCTCTGTGCCCTGTGGTT
AGTCTGGTTAAGGGTTTATCTATCTTGTTGATTTTTCTCAAAGAACCAGCTCCTGGTGTTGTTGATTCTT
TGAATAGTTCTTTTTGTTTCCATTTGGTTGATTTCAGCCCTGAGTTTGATTATTTCCTGCCGTCTACTCC
TCTTGGGTGTATTTGCTTCTTTTTGTTCTAGAGCTTTCAGGTGTGCTGTCCCCGCTGTCGTTGAAACTTT
TTCTTGAGGACTTTGCCCAAAACATTTGCAAGATTTGGAGACTCAGAACCTTTTAACCCCAGCATAGTCT
CTTTCTCGTGGAGAGCAACATCTCCAAATACACCAACAGCATCAGGCAAAAACATAGTGTGATATGACCT
CCTCTGTAGCTGACAGATGACAGAGGAAAGCAGAACAAAAGAGGGGAACATTGCCTCGAGTGAGGGAGTG
AGAAACTCACCACCGTTCTGCAGGCTGACTGGGGACCCAGAGAAGCTGGGGACCCAGAGAAACCTGAAAG
CTTGGGGTCCTTGAGGTGGTTTTCAGTGGGAGTTTGGTCCCACCTCTGTCACCTGCCTGGCCTGAGTTCT
AGATATGACACTCTTTGTTGCTATGAGGAAGTTATCCGTAAACAAAAAGGCCCTTCCATTGTGATGTCCC
ATAAATGCCAACATCCACTGCTACTGTTTATCAATGCACTACTCAGCCATCAGAACTTCCCCAAAGAAGA
TCAGTAAGAACAGCCATCGAAATCCACTTCGATGAAGTTCTGGCCCAGGATAGGAAGGCGAATGGACAGA
CCCAGATCAGTGAATCCATTTGTTTCAGGAGATGAACAGTCTATAAAAACCTCAGTGGCCTCCAAAGGTC
TCAGCCAGCACCTAGCTGGGCCATAAGAGAAGGCAAATGTTTCTAATGACTTCACTGTAGCGTAGACCAC
TTCCTTCAACAGAGCTTCAGGCTGTCAGTCATTTTAACTCTGACAGTGGCATGGGTGTTTTCTGGCTAGA
CCTTTACAACTGAGTAATGGGTGGGTTTGGGTTCCCAGGGTCAGGAGAATGGAGAGGGCCATGATGTCCA
AACCATGGCAGACTCACAGCCGGCTCCCACCGGCTCCGTTCAGATTCTGGGACAGCACTGGCCTAAGACA
AACGGGGGTATGCAGGCACATCAGCCAGGCCAGGACCTCGTGCTGGGCTGGAGTGTGGTGCTGTTTCTTG
GTCTTGCTTTGTTTGATTCTCTTCTCTTCCTGTCTCGCCTGGCAGCACACCAAGTTCTATACATCCGCAG
CCTCCCGCCTGCCGCAGGGACTGCCTGTGCCCAGATTCCGTCTTCCACCCTGTCTGCGGAGACAATGGAG
TCGAGTACCTCTCCCCCTGCCATGCTGGCTGCAGCAGCCTCAACGTGAGCTCAGCAGCTTCTAAGCAACC
AGTAAGGACAACGACGAAGGGGGTCACAGGGGTCGACAGAACCACATGGAGCTTTGGGGCTCAGTGTGTC
ACCTCAAAAACCGTTCCTTGGCCGTCAGTATCAGCGTCTTAGCCCCCTCCACCATCTCTCATCCTTCTCT
GCCCCGGGGTCATGACCAGATGCCCACAGCCCAAAGTCAGTTAAAGATTGGCTGGAGACCAACGAGTTCA
TTCTTGGGAACTTGGCAAAACAACAGAAACAAGTCCGAGCAGTCGCTCTCAGCGGAAACTTGTGACTGAT
ACTAATGGCTCCCTGTGGCAGGGGGGAACTTCTGGGTTCTGGGTTCTGGGTTCAGGCCATATTACAGAGT
CACATGGCCTGAGAGCAGAGGGTCCCCTGTGACTTGGACCTTAGAGGCCAGTGCTGGGAGCAGCAGTCGT
TTCAAGCCCTCCTCTCCCAGGAGTGCCACAGAGTTGGCCTGAAGCCATGAAATTAAGTTCAAATCTAGGA
TTAGAAACAAAATCTCTCTTCACCTGGTCTTCAAGGCCATGTGTCTCCTTCTTAGACCTAGACCTATGGG
GACTTGTCTGACAATATGTGAGGACAGTGACCCCAGGCCTTCTGGGATCAGGAAACAGCAAATGCCTCCT
CCAGGTTTGTCTCTTAGTCAGAAATCCATGTGTGCAGCCACCATGTCAACCAAAAGCCCTGGTCTCACAG
TTTACATCCAGTCCGATAAATGCACACGATAAATGCTGAGGGACTTCCTGAGGTGACTTACCCTTGAGCA
AAGACCAGAAGAATCCTATGGAAACCTGGAAGCCCATTGCACTCGAGGGAAGCACAGGTGCCAGTGCACC
AGAGAAGCTGAGGAAAGGCCTGGTCGGCGCTAAGCGACAAGTGTCACAGCTATCGGGACAAGGGATCAAC
AGGTCCAGAGCTGGGCAGGGCCAGGCAAGCCAGACTTAGGAGGGAGATTATTTCCCCTCTTAACTCCCTC
TGATGAGAACAATGGAACGTCTATGTCATTACTATAGGATGCCCCCATAGGAAGAATGCACCTGTGTGTC
GGCCATGTGTGTTGGCCATGGGCCAGGCTAGCCCCCACTTACCACCTCTTGCGAGGTCACAGAAAAAAGC
TTCCCCTGTCTGTCACTATGGGCAAAGGCCCTTGGCTCTCAGGTAGAGAAGGGTGAGGAGCTAAGACAAG
ATGGCCCCTTAGTTTGCCTCTTAACTGGAAGAGTGGTGGCTTCTGTGGTTTTCTTATCTTTATTTACTCT
CCGCTGACTCATCTACTTCCAGAAATAATCTTGACAAGCTTCCAATAACAGCACAAGTACATCAAGACAA
AATCCATTACCATGATAGCAAGGGAAAAAATAATAAATTATAAGGGGGATAAAGTCTTAGTATTTTAAGA
TCCAGATTGCGGGAGGTTACGGCAATATCGTGTGTTAGGAACTTCTGAGCTTGAGGGCCACAGTCCCACT
GACTGACAGAGGGAGGCCTCCTCTAGCTCATTAGTTGGCTCAGCCCCATAACAGCTCTGACACCAACGTC
ATCATAGGCTGGGCCATCAGCCACTGCAAGGAGGGAAGGCAAAGATGACCAGATGAACGGTGGCCGCCAA
ATGAGTCGAACAGCAGTGTTGAGCCACATAGAATTATGATGAGGCAGGGTCCTCCTCTGGGGGCTCTTCT
TAGGTCAAAGTACACCTCTTCAGTGGGGAGTGCTACAGAGCATTCAAGAGACTCTGTTATTGTTTCCTCT
ACTCCCGGAACATACCTTCTCCCATAACAACTTGCGCAACCACTAGAGCGGGCTGGACCGGGAGGAGAGC
TCCCAGGTTCCAGGCGCTGAGCTGATGAGGGTGCTTTGCCTGGTGATGGGATCTTGGGCATTCAGATGTT
GGAAGACTGCAGGAGTTCATCCCATGCTGGACTCTTGGTGAAGACCATTTCTTTCTTTTTTTTATTTTAA
AGATTTGTTTATTTATTTTATGCATGTGAGTGCACTGTCACTCTCTTCAGACACACCAGAAGAGGGCATC
AGGTCTCATTACAGATGGTTGTGAGCCACCATATGGTTGCTGGGAATTGAACTCAGAACCTCTGGAAGAG
CAGTCAGTGCTCTTAACCGCCGAGCCATCTCTCCAGACCGTGAAGACCATTTCTAATGTCCCCAAAGTGA
AAGAGCCAGGACCCCATGGGGCACTCTAAAACCCTCATCTGACCTCTTCAGTCTTTGAATCCTAGCTTTT
CCTCCATCCCTGGAGGGCTGAAGGGAGGGCAGAGGGATTCGTTCATATTAGAGGGAGGCAGTGTGGATAT
GTGAAACATACAGAGGTTAAATCATGACGTGGCCCCTCCTGTTCTTCAAGATCTATTTGAACTGTAGCTG
TGTGACTGGAGGATCCGCATCGGCCAAGACAGGCTCCTGCCCTACATCCTGTGCACAGCTGCTGCTCCCG
TCCATCTTCCTCATCTCCTTTGTGGCACTCATTGCCTGCGTCTCCCACAACCCCCTCTACATGATGGTCC
TACGGTGAGTGAAATCTCTGAGCCTCTGGCTGCTGCTTCCACACCCTCTCTTTGACAGTACATCCTGTCT
CCTACTCAGAGTCTCTCCTCCCTTAACTGGGGAGTCTTAACAGCAACCTACAAGTGTCTAATGGTGAGAA
GACAATAGGTACACTGATGGCAGGAGTTCCCTGTCCCAGGAGGAGCTCCTGGTAGGGAAGCCTGGCCCAG
GCTCCTAGTATCCATGAAACATAGGCCCTGTTGGCACCCCCATCTTTATTTGTGGGAAGTTAAATCATAG
CCCGGGATGGTGGAACTGTCGAAAGGTTGAGCCGCAGGTTCAAGACAGGCTGTGGGTAAAGATTCACTTG
TTCTTCAATATTAAAAGAGGAGAAATCAGCAAGTCAGAAACTGAGGAAAGTTCCAGAAGCAAAGCGAACA
CTGTGAGAAAGGGCTGAAAACGTGAAATTTGCAGGAGACTGGGGACTCAGTGCTCAGAGTGGAGGGACAA
GGGACCAGGAACCAGGGTGGGGTCAAGAGAAGTGTCTGGTTTTTAATAGAGAGCGTTCCCTCTTCTCATT
CTACCAAGAAGGAAAATGTAAGAAAAGAAAAAGAAAAAAGAAAAGAAATCTCACCCAAGTTCATCTAACC
ACCTCCTGCCTGCCTCTCTGACCTCCCATCCAGCCCCATCAGCTTACTTGGAAAGCAGAGAAGGAGAAAC
ACAAAGGTTGAAAACTCCCTATCAGATTAAAAGTCTCTACTTGAGTGTCTTGGGTGTTGCCTGACCTCAT
GACTCCCAGTTTCCTTGACTATGAAAGGAAGATAGTGATACCCACCTCCAAGTGTGAGTGTGAGCAATTA
CAAGGTCAATCGCTTCGTAAACAGAAGCTCCTCTGCTGGGCGTGGTGGTGTCATCTATAAATCTAGTGCT
TGGAAGTAGGAGAACCAAGAGTTCAAGGTCTTCTTTGCCTAGATAGTAAGTTACAAGCCAGCCTAAGCAA
TATGAGACCCTACCTTACAAGGTTCTCAATGTTTGAGTAACAATCCCTTTGGGGGTTGACTGACTCTTTC
ACAGGGGTTGCCTACTACGACCATCAGAAAACACAGAATTTACATTACAATTCATAACATTAGCAAAATT
AAAATTAGGAAGTAACAACGAAATAATTTAGTGGTTGGGAGGCTCACCACAGCATAAGGAACTGTATTAA
AGGGTCGCAACCTTAGGAAGGTTGAAGACCACTGCCCTACCTCAGAAAAGACATGGGGGCTGGTGTTCTC
CATATTGATTAGGACCTATCTCTCCAGAAGGCTAAGCAATGAGGAAGGACCAGCTGCCATGTCAATAATG
TAGCTTAGTAACAGAGCACTTGGTTAGCACATGTATTTTACTCTATCCAAGTCTAGTCTCTTTCCATTCA
TCCCAGAACCTCAAACTACTAAATAGTACCATGAACATTCAGGATTACGTCCTCCCAACTTAGTCAAATC
CCTCAGGATACATCTTCATGGATACACCAAGAGATTTGTTTCCCAGGTCATTCCAGATCCATACGTTAGC
AATGAAGATTGGCCATCGAAGTCATCCATTATGGATCGGGCCATTGGAGACAAGCTGATACAGAGGGAAA
GAACATCCATTCAACTGGAATTCCAGGCACTGTCCACTGGGAGATCCCACATGGTCCCTCTTTCTCCTAC
TTGAACTCCAAAGAGCCTCCCTTCACTGGGTCATCCCCCTGGGGATGTCTCTCAGATGCCGACTAACTTT
GGCAATGGTTGTAAGATAATATGGAATGATCTGGAGGCCACTTTTGAGTTCTTTTCCACTCTGTCCTCGG
CTCCAAATTCTTTTCCAAATGATAATCACCCAGCCAGGAGTATGTGGTGTGAAAACGTGGAGAAGCCAGA
GTCTGGTCACACAGCAGGCGCTTCTACCACTGCTCTGAGCTCCTCAACCCTGTGTGTCAGATGCCAACCC
CCTTTTCACTCTCCAGTTGAGGTGGCAGCTCTCTAGGGAGAGCTGAACCTCTTTACCTGGCTTTGCCATC
CCCCTCTTGAAGGAGAAAAGGTAGAAAGCAGGACCACGCACAGAATCCACAGGACCCAATGCAAATGAAA
CTGTAAAGACTTTGGCTTGTGAATCATTTAAGAACTCCAAGACATACGTAAACCAAAGCTGGGCCTTTCT
GACGGCAGCATCCTATGCAGCTATTCAAGTCACACCTTACAAAGCTGGCCCAGGGCAAGGAAATGTTTCA
GATCTGCACAGAAGCTGATAGCCCAGGGCCCTCTTTTCCTGGTACAAGATATTCTGTGGGTGTTGTTCAG
ACCTGCTGTCTAAGCATCCAGCCAGAAATTCCTTTCTACTTGGTCTCTCTTGCCTGCTTTCTTTCCAAGA
AGTCTCTGGACAGTCCTGAAAACAATACAGACTATTGGTTCTCAAACCCAAGTTTACATCCAACTCTCCT
GGAATATCCACGTTTTTGATAGATGCCAGGCGGAGAATCTGTATTTTGAACGTAGCCTGGGCGGTGCCTC
TGATCCAGCAACAGTGCTTTGAGAACGATGGGTATAACAGGCATTCAGCAGATAATGCGCATCATCCATG
TGTCTGGTCAGACTCCATGAGGACCAGTGCTGACCTGCTCTGAGTCTTTCTACCCCCTACACCTCATACC
CTCCAGGTGATGCTGCAGTCTAGAGTCCTACTGGTGCTAACTTCCTGTTTTCCTTTTCAGTGTGGTGAAC
CAGGATGAAAAGTCGTTTGCCATTGGAGTACAGTTCTTGTTGATGCGTTTGCTGGGTAGGTGATTTAGGC
CCTGGACCGTGGGCATCTAAAAGCCATGTGGGGCGGGGCAGGGTTTGGAGAAGTCCCAGTTCATCTTGGC
TGCATATTCCTAACAGCAAGAAGAACCAAACCCTGACAGATGACAGTTTGCCTGATTGCTTGAGATCGGG
GAAGATTCTGTCTACAGTATTCAAGCCAAATTTAAATTATGTAAGAAAAAGAAAAGGTCATAGCTGAGAG
TGCTGAATACAGAGTTAAAGGCAGGTATTTTTAAATCTGTCTCCAAGAATCCGCCCTAGGAAACAAGACC
TTGCATTCCTGCCCTAGACAGAGCAGTTTCTGAGCTGAGCCCTGGCTTCCCTCCCATTTTCCAAACCTCA
GTCCTCTGCCACGGTTCAGCAAATGTCTCTTCTGTCAGGGGATCCTGCCTCCAAACCCTGTCTGTCCTCA
AGTAGCCTGGAGCCCCCAGCTCAACACTCAAAATACTCAAGCTGTGCATACACATAATCAGCAGAGACCT
ACAGGGAGAGAGAACTCCCACGGGAGCAGGAGGCATCTGGCTTCCCTGAGTGCAGGGACTGCAGGGAGAG
GAGACACACAGTAGGTTCCTTCCCAGCTGGCCAACTCTCATCTGCCCGTTGACATCCATGGCACAGTCAT
GCATTAGACTGAACCCCAGGCACTGATGTGGAGGTCTTGCTACAATGGATGAACAAAAGTATAAAGATTG
AATTTAACAGAGGATCTTTGTACTAAGAATTTCAGGGGTGGGGATTTTGAGGTTCCGGGCAGAAGGACTT
GAGTTACTATCAATGTGAGCCTTCCTACTTTAACCACTGACACCCTCTGCATTATTATACAGGGGTGCAG
GTCACATCTTGCCCTGCCATGGCTTCCATTAGCCTTCCAGCGTGCGTCATCATCCCAGCTGAAGTCAGAT
GGGCAGGTGCCTATGCAGGGTCTGAGCCAGCATTTTCAATGGAGTTTCTTAGCCGCAAGCCATTTATCCT
ATCATGAAGCGTCTCAGTTCCAAAGGACCCTTAACCCTCTAAATGACAACCGTCACATTCCTGGAACCAC
ACAGGGGACACAGGGTATCTTGTCCAGGTTGAGGCTGGTTCATTAGCACAGCTGCTTGTATTTATTTATT
TATGACTTACCATCTATAGCTGGTTTATCACTTACTCCAATGCCCTGGCGGGGAAAGAGCCCTGGCATTC
CTCCCAGAGGACCTTCTGCCCTGCTTCTATTGGTGACTTAGAGAATGGCCCCTCTGCCCCGCTGCTCCAA
GCCCTTGCTGCTTCCTCTTTCCCACCTGAGCTCAGCTAACTCTGTGGACCACAGTTGGAAAACACAGGCC
TAGAGGAAATCCACTGGTCACTTAAGAACACAGCTTCACTCTGGGTTAGATGAAAAGACTGCCTATTTAT
AAACCCCAGCAGGCTCACAGTCACCCACTGAGATGTGGTTGAAAACTCTCCTTGACTTCTTTTCTATTTA
GGGATGGGTCACTAGGGCCAGTTTCCAGCCACTGTTCACCTGGAAACTGGAGTGTGAGCAGGGGCTGAGG
GGCTGCTCTTGGGAATTTCTCTATGTAGGTAGGGAACAGGACTGGGTTGCCTCTTTCTTCCAGAAACAGG
AACTCACAGGGTGCTAGAGTGTCTGGTGTTTTCAGCACCGCGTTTCTGAGAATGGGATGGCCCATCTGCA
ACGTAAATCCTGGACAAGTCCACCCTGATCCACTGACTGCTGGACAAGTCCACTCTGACCCACTGGCGGC
TAGACAAGTCCACCCTGACCCGTTGGCTGCTCACAACGTGGCTAGCACCACTGAGTTTGGTGGGGTTGGG
TAGTGGGCATCTAAAATATTCATCACCCAGTAAGGTGTTGTGCCTGCTGGACTTCATCTTAACATTCAGA
TGCTAGGATGATTTGGCAACCTGTGTTTCTGAGCAAGACGGGGAACTAAAGGTCTCCAAAGGGTGTTGTC
CGATCTAGTGGGCTAGTTTGGAGTGGGAGAAGAACGTGGGATTTGAACTTCAGTGAACCCTTGGGTCCTC
TTGTTCGCTTGCACTTTATTCCAGTGACTCCACCCACCCACAGAGGCTGGGGGAGTCCTTGGAGGCGTTA
GCACTCTCGAGATGGAACTTCTACCTGGGGCCAGCCTTGAGCTTGAGAACAGTGCTGTGTGTCTGAGCTG
ACTCCAGGTTGAGCCCGTGATTCATTCTGCCCTGAAAGAATGGATTCCCTTTTATCTCCTAAGCCTTCTG
GTGAAAGGGGAACTCTTGCTGAGCTTTGCTGCAGCTTTCTGCTTTCCCGAGTCACTGTGATTCCGAGGGC
AATGTGTGAACCGTGCCATTGGTGTGGGGGGCTCGGACATCTCTGACTGCAGATCCAAGCCACCCAGACA
CCCTGAATTCTCTTTCCCTTTGTTGCCAGCCTGGCTGCCATCTCCATCCCTTTATGGCCTGCTCATCGAC
TCCTCCTGTATCCGGTGGAACTACCTATGCTCAGGGAGACGAGGGGCCTGTGCGTATTATGACAACGATG
CTCTTCGAAACAGGTGAGAGCTCCCACATACACAGGTGTCTAGGAGATTAGGTCTGTTGTCCCAGCACGG
GGGGGGGGGGGAAAGCCCTTTTTTTAAACCTCATGGATGTGGTGGAGGCTGTCAGCTTTCTAAGGGGCAA
AATAGGAGGGAATGTCTCTATAGATTCTGCCATGGACAGAGAGCAGAGGCTACCATAGCAGGCATTTGGG
GACCTATAAGGAGCCATTCAGGTGAGGAGACACAGGGTCTGCTTTGAGGTGTCCCCCAAAGTACTGTCCC
TCACAAGCCTACTCATAGCAGCAGACAATGGTCACCAAAGTTAAAGGTGTACCTGGAGCCTGAGAGAGAT
TTCGAATATGGAAAAGATACCAGTCACTCTCCACTGCTCAGAGAATGGGGAACACAGCACGGCGCCCAGG
AGAGGTCCAAAGAAGCAGGAGTCCAACCTATGCCTGTGAGCCAAATGCAGCCATCTCTTCTGTTGCTGCC
GTTCCATCATCTAGAAATGGTGGAGAAGGTGGGCCTGCAGGGGCTAGAAATACTGAGGACGGATAGAAAA
GGCCAGGTTAGAGAAGGCCAGCAGAGAGGCAGCTGAAAGATACAGACCCTCTGGAGACAGCTGGGACAGT
AGTGTCACCTCTGGCCATCAGTGATGTCACAGGAGAGCAGAGGATCACTAGCGTCATGTGTCTCTCCACA
GTGACCACCTGAGAAGTGACAGCTCGTCACCTGAGTAACGATCTTCACAGCTTCTAGTGAACTGTGAAAA
CAAAGATCATCTTCCCGCTGCCCCTGGGCCTCACCATACCTGTCTCTTCCTGCCTCATAGGTACCTGGGC
CTACAGGTAATCTACAAGGTCTTGGGCACACTGCTGCTCTTCTTCATCAGCTGGAGGGTGAAGAAGAACA
GGGAATACAGTCTGCAGGAGAATGCCTCGGGCCTCATCTGACCTTCAGCTGGGACTACTGCCCTGCCCCA
GAGACTGGATATCCTACCCCTCCACACCTACCTATATTAACTAATGTTAGCATGCCTTCCTCCTCCTTCC
CTCTCTCTCTCCCTCTCTCTTGCGCACACACACACACACACACATACACACATACACACACATGCGCCAG
TGCGCGCACAAAAGAGAGAGAGAGAGAGAGTTAGTTCACTCACCCCTTGTCTCACCGGCCTCTTCCTCCT
AAAGTCTCCCGCATGGTTCTTAGGGCCTGAAGGGTCCTGAGCTGAATGAGCGCTGGACTGAAGGGTCTAG
TTTCTCAGAGTCCTTGGAAGGGATCCTCTCCTCAGCCCTGGGTCTTCCACTGTCCGTACTAAGGCAGCCT
CTGTTCTCACCAGTCTCTCAGGAACACTTCAGTGGTGGGAGAAATGGGAGCCTTGACTTCCGTGCCAGGC
AGCCCAGCTCCTCTGTCTCTGCCCACACCCAACAGAGAGGCAGGGGAGCAGCAGGTGGTTGTTTCTTAGC
AGCTACCACTGGGCTAAGGGAGCTTGGCTGTGGCCGCTTTGCATCTGGCTTCAGTCTAGCATGGAACCCA
GGAGGGACCTATCTCCCTTTCCTTTCCCACTTTCTGCCCTCAAGGCTGATTTGGAAACCACCACTTTGGG
AAGTATAACCCTCTCTGTATACTACTCCCTGGTGTCAGAGCAGGAACGAAGGAACCAGCAAGGCTAGGTC
ATCCAAGCACTGTTCCTAGGAACCTGAGGCCTACCCTCAGGTCAGCATGTGCTTCCTTCTGCCTTTCAAA
CCAACAAAGCCCATGTAAGGAGCCCATGTAAGGAGGACCCAGTCTCCCACCCTCACAGTAGAGACAAGGT
CTTTCCCCCTTCAGAGACGCAAATAGCCATCTTTACCATGCCCCGGAAACAAAAGAGTGACCTCACATGG
GCTTTGCTGTCTGGACTTCAGCTCATGTGCAGAGGAGTAAAGGCGGTGAATGGGGGGTGTGATCCTGAGA
TGCAGTCAGACCAAGGAATGGAGCAGAGTGGATGCCACCTGTCACTGTGCCAGATGGCAAGTTGCAGCCC
ACAAAGAGAGTAGCCGGGTTGACTCGCTTGCAAACACATTGTCTTCGTTGGCATTTTCTCAGCTTTTCCT
CACTACCTCTCTGGATGTGGAGGTGGGCTCCTTCTGTGGGCCTGGGCTGGGGGCCACCACTGCCATGCAG
TGGAGAGGCAAAGGCAGAGGCCCAGGAATGAGGCCTTTTCTCAACGATTCTGTTTATCAGAGAACTGGCT
TGGTGTTTGTCAGGGATGCTGTATGGTTCTCTGAGAGCTCCCCTGGAAAACCTTACCTCCATTCGCATTG
GGATTGCTCCCAGAGAGCGCTCGGTGGGCAGCAAGAACATGCTCACACACTTGCCAAGCATATCCAGAAC
CAAAAAGGCTGGCGGAAAAGATCCTGGTCTGACTGCCCATGCACACCCTGAGGCTCCAGCAGGTTGACTG
TGTGCCTGAGCTGGAGATCTGGCTCACCGTGTCCCCGAAGCAGCTAGGCAATTGACATTGCCCTATGTGA
CATTTAAGTGCCTTTCAGAAATGCCTTATATGGTGAGAAGCCAAATGGTTTATGTCGGCGTGGCAAAGGT
CCTGTGAGACTGAAGCCTTCAGGGAGCCTTTGTTACTGGCAGTGTTCTTTCTGAAGCCACCGGGCTCATT
TCCACAGATCTTATTAAGGAGGAGGCTCCACACAACCTCACGGGCCAGTTTCAGCATGTGGAACGGTTCT
GCTTGGAACCTGATTCTCTGCAACTAACCCTGGGCTTACTCACTGCAGTTTAACCAGATTCTGAAGGCAT
AGCCTTCCACCAATCTCGCAAACAATGAACGTCAACCTTCCGTTCCCGGAAGATCAGCTGTCCCCACAAG
TCTTTATTCTATTATATTGCTATTAATATATTATAATTCTGTTCTGGGTGT

An exemplary DNA sequence of the Slco2a1 gene in humans is provided below.

(SEQ ID NO: 21)

CCTCCGGCCACTGCCGCCGCGGTCGCCTCTCACCCGCCCGGCCGCTCCAG
CCCGAGGCGCCCCGACCCCGCGCCACTCCGCGCCCGGCCAGCCGCCCGCA
GCCATGGGGCTCCTGCCCAAGCTCGGCGCGTCCCAGGGCAGCGACACCTC
TACTAGCCGAGCCGGCCGCTGTGCCCGCTCGGTCTTCGGCAACATTAAGG
TGTTTGTGCTCTGCCAAGGCCTCCTGCAGCTCTGCCAACTCCTGTACAGC
GCCTACTTCAAGAGCAGCCTCACCACCATTGAGAAGCGCTTTGGGCTCTC
CAGTTCTTCATCGGGTCTCATTTCCAGCTTGAATGAGATCAGCAATGCCA
TCCTCATCATCTTTGTCAGCTACTTTGGCAGCCGGGTGCACCGTCCACGT
CTGATTGGCATCGGAGGTCTCTTCCTGGCTGCAGGTGCCTTCATCCTCAC
CCTCCCACACTTCCTCTCCGAGCCCTACCAGTACACCTTGGCCAGCACTG
GGAACAACAGCCGCTTGCAGGCCGAGCTCTGCCAGAAGCATTGGCAGGAC
CTGCCTCCCAGTAAGTGCCACAGCACCACCCAGAACCCCCAGAAGGAGAC
CAGCAGCATGTGGGGCCTGATGGTGGTTGCCCAGCTGCTGGCTGGCATCG
GGACAGTGCCTATTCAGCCATTTGGGATCTCCTATGTGGATGACTTCTCA
GAGCCCAGCAACTCGCCCCTGTACATCTCCATCTTATTTGCCATCTCTGT
ATTTGGACCGGCTTTCGGGTACCTGCTGGGCTCTGTCATGCTGCAGATCT
TTGTGGACTATGGCAGGGTCAACACAGCTGCAGTTAACTTGGTCCCGGGT
GACCCCCGATGGATTGGAGCCTGGTGGCTAGGCCTGCTCATTTCTTCAGC
TTTATTGGTTCTCACCTCTTTCCCCTTTTTTTTCTTCCCTCGAGCAATGC
CCATAGGAGCAAAGAGGGCTCCTGCCACAGCAGATGAAGCAAGGAAGTTG
GAGGAGGCCAAGTCAAGAGGCTCCCTGGTGGATTTCATTAAACGGTTTCC
ATGCATCTTTCTGAGGCTCCTGATGAACTCACTCTTCGTCCTGGTGGTCC
TGGCCCAGTGCACCTTCTCCTCCGTCATTGCTGGCCTCTCCACCTTCCTC
AACAAGTTCCTGGAGAAGCAGTATGGCACCTCAGCAGCCTATGCCAACTT
CCTCATTGGTGCTGTGAACCTCCCTGCTGCAGCCTTGGGGATGCTGTTTG
GAGGAATCCTCATGAAGCGCTTTGTTTTCTCTCTACAAGCCATTCCCCGC
ATAGCTACCACCATCATCACCATCTCCATGATCCTTTGTGTTCCTTTGTT
CTTCATGGGATGCTCCACCCCAACTGTGGCCGAAGTCTACCCCCCTAGCA
CATCAAGTTCTATACATCCGCAGTCTCCTGCCTGCCGCAGGGACTGCTCG
TGCCCAGATTCTATCTTCCACCCGGTCTGTGGAGACAATGGAATCGAGTA
CCTCTCCCCTTGCCATGCCGGCTGCAGCAACATCAACATGAGCTCTGCAA
CCTCCAAGCAACTGATCTATTTGAACTGCAGCTGTGTGACCGGGGGATCC
GCTTCAGCAAAGACAGGATCGTGCCCTGTCCCCTGTGCCCACTTCCTGCT
CCCGGCCATCTTCCTCATCTCCTTCGTGTCCCTGATAGCCTGCATCTCCC
ACAACCCCCTCTACATGATGGTTCTGCGTGTGGTGAACCAGGAGGAAAAG
TCATTTGCCATCGGGGTGCAGTTCTTGTTGATGCGCTTGCTGGCCTGGCT
GCCATCTCCAGCCCTCTATGGCCTCACCATTGACCACTCCTGCATCCGGT
GGAACTCGCTGTGCTTGGGGAGGCGAGGGGCCTGCGCCTACTATGACAAC
GATGCTCTCCGAGACAGGTACCTGGGCCTGCAGATGGGCTACAAGGCGCT
GGGCATGCTGCTGCTTTGCTTCATCAGCTGGAGGGTGAAGAAGAACAAGG
AGTACAACGTGCAGAAGGCGGCAGGCCTCATCTGACCCCACCCTGGGCCA
CTGCCCTGCTCCAGAGAGTGGACCTTGACTCTTCCACACCTGCCTATACT
CACTAATGTTAACACGTCATTTCCTTTTTGTATTTTTAAACAAGAAAGAA
AACCCCAGTCCTCATTTGCCTTCCCTACCTCTTCCTCCCAGAGTCCTCCC
CACAGTTCCTAAGGGCCACTGTGTACCCGGGCTGTGTGGGCCAGAACTGG
GGGGCTGAGTCTTCCCTGGCCCCTTGGAAGAGGCCCCCAGATGCCCAGGC
TCACTTCAGTGTTGAGTCCTCCATTGAGGATGCCCACTGAGGCAGCCAGG
CCCCTCACCAGCCCTGGGGGGAATCCTAAACAGAGAGAGAAAAAGGGTAT
CTGCCCTTCTTGCCAGGCAGCTCCACTCTCCCGCTGACTGCCCACACCCT
GCAGAGTGGCAGGGGTGAAAGGAAGAAGGAAGTGGCTGAGTTATTAATAG
CCAGAGCCACTGGGAGACTGGGGAGACTGGCTGTAACCCCCTTCACACCT
GGGTTTGGCATCAGCACAGACTACGGGAGGGGCTGGCTCCCTCCCCCTCA
GACCCTCACTTCCTGTACCTAGAGGCCATTCTGGATGCTGCCATGTTGGG
AAGTACAGTCTCTGCCCATTACCTGCATGCAGGCACCAGAGCAGGGACTG
AGAAACCCCAAGGATGGGTCATCTAAGTGCTGTCCATATGAACCCTGGAC
TTTCTGTCCTTAGATCCTCACATGTTATCCCTGTCTTTCTGGGGTACGTT
TCAAACTGAGGAAGCTACAACACAGTGAAGACCCAAGGAAGGCCTATGAA
ATGGTCCTGATGCCCAACCTCCCACCCCTTCAATGTGGGGACGAGACCCC
CTCATCTCAGAGTAATGGGAAGAACCTCCCACATCTCCCTGGCAGCAGAT
GAGGTGGCTTCACATGCACTTCCCTGTCTGGACTTCAGCCCGTATTCCGA
GGAGTAGAGAGGCAGAAGAGATGTCAGCAAAGCAAGTGATGAAGCAGAGT
GGATGTCCACTGTCACCAAGCTGGATGGCAAGCTGCGGCCCACAAAACAG
CCAGTCAGGTTGGCTTTCCTGGTTTCAGACATGCTCATACCATTCCCATT
TTCTCAGCCTCTTCTCACTGCCTCCAGAGAGGTGGGTGCCTGGGTTGAGA
GACACAGCTGCTACGTGATAGATGTTGAGAGACAGAAGCCAACGAAGGAG
GTCATTCATCAACAAATATATTTATTGGAGACCGACTTTGTGCAAAGCAA
TGCTAATCAGGGTTCTCCATGGAGCTTCCCTCAGCTCTTACCTCACCTCC
CTCCATTTACATTAGGGCCTTCTCCCAGGGTGTGCTCGGTGGGCAGTGTG
GGACTGGGGGTGTGGGAGTTGGTGAGAGCAGGAGGAGAGGTGGGGACAGC
AAGAAGCCACAGATTGGCATGAAGGATCCTGACCTGACTATCCATGCCAT
CCATGGCCCCCAGACTGACTCTGCACCTGGCCCTTTGCCAGACAGCTCTG
TCTCCCCATGTCCTCTGGAACAGCTGGGCATGGGTCATGGCCATTCATGA
CCCTTAAGTGCCACCCTTCTTGGAAGACCCCCTCCAGAAGCATACTGGAA
GCCACCTCTGGAAAAGCCTCATATGGTGATATGCCAAAATATTTATGTCA
ATGTCCAAACAAAGTCCAATGCCATGAGACTGAAGTCTTTGTGGAAACCA
CTGTTACAGACAAGCTTATTTCCAAAGCCACCTCATTTCCAAACATCTCA
CTCAGGAAGGGAGGCTCAATGTAACCTCAGGGGCCAGTTTTAGCATTTGA
AATGGTTCTGCTTGGAAAATGATGCCCTGCAACTAACCCTGGTCTTTCCC
ATGGCAATTTAACCACATTTGGAAGGCACTGCCTTCAGCTGAGTTTATGA
ACAATGAATGCCAACCTTCAGGTTCTAGAAGATTGGTTGCACTCCCAAAC
CTTTATTCTATTATATTACTATTAAAATATTCTAATTTTGCTATTGAAAA
AAAAAAAAAAAAAAAAAAAAAAAAAAAAA

Srprb, or signal recognition particle receptor, B subunit, is a component of the SRP (signal recognition particle) receptor that plays a role in correct targeting of the nascent secretory proteins to the endoplasmic reticulum membrane system. The Srprb cDNA sequence in Mus musculus is provided in GenBank Accession No. BC003798.1 (GI:13277830), incorporated herein by reference. The Srprb amino acid sequence in Mus musculus is provided in GenBank Accession No. AAH03798.1 (GI:13277831), incorporated herein by reference. The Srprb cDNA sequence in Homo sapiens is provided in GenBank Accession No. BC063001.1 (GI:38649414), incorporated herein by reference. The Srprb amino acid sequence in Homo sapiens is provided in GenBank Accession No. AAH63001.1 (GI:38649415), incorporated herein by reference.

An exemplary DNA sequence of the Srprb gene in Mus musculus strain C57BL/6J chromosome 9, GRCm38.p1 C57BL/6J is provided below.

NC_000075.6 (GI: 372099101: c103202065-103188033):
(SEQ ID NO: 8)
TCTTTCATGGCTTCGGCGAACACCCGGCGAGTTGGCGATGGCGCCGGTGGCGCCTTCCAGCCTTACCTCG
ACTCCTTACGACAGGAGCTGCAGCAGAGGGATCCGACGCTGCTCTCCGTGGCGGTGGCTCTCCTGGCGGT
GCTGCTGACGCTGGGTGAGGCGGCCGGCATTCACGGCGAGGGCAGGGTGGCTCGGCGCCGCAGGCCAGGG
AGGCGGACGGCTGCGTTCCCTCTTCTACTACTCGTCTTCCAGACCCAGCAGTGTGGAAAGGGGACGCTGA
AGTGACCTGGGTCTTGGGACAGGACTTACATGAGGCGGCCCTGTGCGCTTGAGCGCTGCGGCCGCCATCG
TTCATTCCAACTTTGAGCCACTTGTGTCTGAACTGGGCTCTGAGATACTGGGGTACTAAGGTAGAGTAGA
AATCCACCAATCAAATGTAGCAAGCCACTAATTTTTCCCAGATCCATCATTCTGTGTATACTTTTAGTCT
CCACATTTGGATGAGGGAAATAGACGTGAAAAGGTTTACAGTTGATAGTGGCTGGCGCTCCGTGAGTTCC
TAACAAAACTGACTCTAATCCGTGTGACAATGAAAGTAGTCCAGATGGACGCTTTGTTTTTTTGTCGTAA
ATGCCTACTAATAATGTCTTTAGAAAATGAAGGCTGCCTTTCTCTTCTCTGCAGTGTTCTGGAAGTTCAT
CTGGAGCCGAAAGAGCAGTCAGAGAGCTGTTCTCTTCGTTGGTCTCTGTGACTCTGGGAAAACGTTGCTG
TTTGTCAGGGTAATGCTTTGATTTTCACCTCGGTTAGACTTAAGAGGGATGGGTAAGGCATTTCCGTAGC
CCTGTCATCTTGCAGTTTATTTGGTACAGCAAGTTTAGGACATGTTTTTAAAAATATTTTTTTTTCTTTT
TAATTATATGTATGTGTGTGGGTATATGCACATGAGTGCCTGTACCTGTAGAGGCCAGATGTGTCAGATT
TTGTAGAGTCGGTCTTCAAAGCTGCCTGATGTGGGTGCTGGGAATTGAATTCAGGTCCCTTTGCTAGAAG
CAGGATGGGATCTTATCCAACTTAGTTGAGTCTCCAGCCCTGGGAGTTAATTCTTTTTTTTTTTTTAAGA
TTTATTTATTATTATATCTAAGTACACTGTAGCTGTCTTCAGATGCACCAGAAGAGGGAGTCAGATCTCA
TTACAGATGGTTGTGAGCCACCATGTGGTTGCTGGGATTTGAACTCAGGACCTTCGGAAGAGCAGTCGGT
GCTCTTAACCACTGAGCTATCTCTCCAGCCCCCTGGGAGTTATATAAAAAAGACTCTAGGAAAACCTGTA
AAAACACACACACACACACACACACACACACATACACACACGATGCTGTTAGAATACGTGGTCTGGGGAT
GGGGAGGTGACTCAGTCTGTTTTCTTGCCTGGCAAAAAAATGAGGGTCTGAGTTTGGATCGGTGGCACAT
ACGTGAAAGCCAGGTCCAGAAGTTCTTGTCAGCAATCCCAGGGCCTAGGAGGTATGGATCCCTGGAATTC
CTTGGCTCACCACCACCCATGCTAGTTGGTTTTCTCCCCGGGTTCAGTGAGAGACCACAGCCTCACAAAA
GGTGGCTGGCTGTGTGGTGGTGGTCCATGCCTTTAAAGCCAACACTTGGGAGGCAGAAGTAGGAGGATCT
CTTCAAGTTTGAGGCCAGCTTGGTCTGTAGAATGAGTTCCAGGACAGCCAGAGCTGCACAGAGAAACCCT
ATGTCAGGGAAAAAAAAAAAAAAAAAAAAGCAAAAACAGTAAATAAATAAGGTGGAGAAAGATTAAGGAC
TCCCGACATCAACCAACCTTCAGTGTACATACACCTGTTAAAAAATGCATTACCAGTTTCTTGGGCTACT
CAGAATATAATGGTTATGGACATACATTTTTTTGTTTGTTTGTTTGATTTTGTTTTTTTCCAGACAGGGT
TTCTCTGTATAGCCCTGGCTGTCCTGGAACTCACTCTGTAGACCAGGCTGGTCTTGAACTCAGAAATCCA
CCTGCCTCTGCCTCCTAAGTGCTGGGATTAAAGGCCTGTGCCACCACTGCCTGGATAGACATATATTCTT
AAGTGTAGCTTGCGTTCTTATTCTTTCTTTAAACAATCAATAAAGGATAAAGGAAGCCCAATAACTTTTG
CTGATTTCTGTGACTTAAAATAGTCCCAGCATGGCTAGTTCTGAGCAATCCAGCATTATCACAATCATGA
CAAAAATTACCCTCCTTCGACAGTCTTAAGCTAGAGAGGCCTCCCTTTACTAACACTTAGTTTTATGTAA
GCCAGTGTATTTTAACTGAACATGTTTTACTATGGAGAATTTACCCTCTTCCTTTGGAGGGTATAAATAA
TAATAATAGTAGTAGTAGTAGTAGTAATAGCTATTATTATTTTAATTGTGTGTGTGTGTTCATTCATGTG
TGTAAGGGCTGGTACGTGTGAGCCTTGGCACATCTGTGGAGGTCAGAGGACCAGAAGTGTTGGTCTTCTC
TCATTGCTGTGCCAGGCAAGCTAGTATGAATTTCTGGAGATTGTCCTGTCTCCCTCCCATTTTCCCCATA
GGAGTACTGGGCTTGTGAATGTGTGTACTACTAGATCTGGGTTTATGTGGGTTCAGAGGGGCTGAACTTA
GGTCATCAAGGTTACACAGGAAGTGTTATGTCCACTGAACATCTCCTGAGGCCTTGAGTTGGTTTTGGTG
CCTAAAATCTTTACAGGTCAGTAATTTGGCCCCTTGCTGTTGTTTAAAAATAATACTAATAATGTTGGGC
CCAGGGTGCCACTTTCTGATGGCACTGCAAAAAGAAGGAATAAAGCTTGTAATTAATTATTATTATTATT
TGGTTTTTCGAGACAGGGTTTCTCTGTGTAGCCCTGGCTGTCCTGGAACTCACTCTGTAGACCAGGCTGG
CCTCGAACTCAAAAATCCGCCTGCCTCTGCTTCCCGAGTGCTGGGATTAAAGGCGTGTGCCATCACTGCC
CGGCTGTAATTAATCACTATTCTACAAGTTAAAAATAAAGCAGTATGATCGTTTTCAGCTGTTTCAAGTA
GCATTCGTTCATTTGACCTTACTCTCCACAGAATGAAAATGTCATTTTTTACATTGCATTCAGTGTTATT
GTAACTCGCTTACCCTGATTTGTCTGTCTTGGTTTTGTTTTGTTTACAGTTGCTAACTGGCCAGTACAGA
GACACACAGACTTCCATTACCGATAGTTCTGCCATATACAAAGTCAACAATAACAGGGTAAGAGGTATCT
GGAATGTTCTAGAATCTGACACTCTTCTTTACTAAGTTCTACTGTCATGTGTGTTGCTGACCCAAAGCAA
CTTGGGGAGGAAAGGATTTAATCTGTTTACACTCCCGGGATGTAGTTCATTGTTAAGGGAATTCAGGGCA
GGGTCCTGGCAGCAGAAGCCATGCAGAAATGCTGCTTGCTGGTTCACTCTTCAGTTCACTTAGCTAGATT
TCTTCATAGCTCCGGACTACCTGCCTAAACATGGCGCCTCCCACATTGGACTCAAGAAAATGACCCATAG
ACATGCACACAGGCCAGTCTCATGGAGGGACTTCTGTTGAGGCTTCTCTTGCATGGCTGGAAGCTGTGTC
AAGTTAACAAAGCTTACTGGGGCACTGTGGCTTGTCAAGGAAGAACCTTCTTGGATGGAGTAGTCTGAAG
ATGTAGGTACCTTTTGTGTAGCTTCATTTTACGTAGAACATATTCTGTATAACAAACAGATCTGCCCTCG
ATAATATAGGTAGCAGATTTAGTCCTGCCCACCCATCCTCGTAACTTACGTACAGGGTATAAGACATTTT
ATTTTTAGGTTCTGTCTGTCCAACATTACTATGAGGTTCAGTAAGTACTGGGTCTTTTTCGGGTATAAGG
AGGCAGTATGTAGACACAGGACAATCATTTGGCTAAAAGTAGTTGTCTTTTTCTGCCTCTGCTAGCCAGA
CTTCTCTTTAGAAGCCGTCCCATAGTTAGATTTTTATTACCCAGAGCCTTGTTAATGTCTAGTGCCTTTT
TTTTTAATTTAAAAAATTTATTTATTTATTTGTTTGTTTGTTTTTGAGACAGGGTTTCTCTGTATAGCTC
CGGCTGTCCTGGAACTCACTCTGTAGACCAGGCTGGCCTCGAACTCAGAAATCCTTCTGCCTCTGCCTCC
CGAGTGCTGGGATTAAAACTTTTTTTCTGTGCCTCACATATACTAGGCAAGCCACTACCCTTCCTTCACC
TGTGTTTACTATTAAGACATCCTCAACAGAAATCCATCTGACTTGCTGCCTGCAGTAAACATTTGCTTGA
TTCATGAGCTCAGCTCCAGTGTGCTGATCTGATGGTGAAGATCTATGCTATGTGAACCTGGAGCTGGACC
GAATGGTGATGTGATGTCACTCTTGCTGTCCTCAGGGTAACAGCCTGACTCTGATCGACCTCCCCGGGCA
TGAGAGCTTGAGGTTTCAGCTCTTAGATCGCTTTAAGTCTTCAGCTAGGTAAGTCAGGGATGAAGTGACA
TGGGTCCCTTCAAGTCTGCACTGCGTCCATGCGCACGCTCACGCTGGTTCCTCTGCCAAGACTTTTGACA
CTGGTATGGGATGAACCTTGGAGTGGCCATTTCTGGGGTGCTAGAACTCGGAAGACGTAGGAGCCTGCTT
AAGGCTGGCTTTTCAGATTGCGCAGGGGGTGTCACTCTACTAGCATGATTAAACTTCAACTATGTGTATG
TGGGGCAGGGTATGTGCACTTGTGTGAGGTACAGCATAGGAAGGGAGGGTGTTGGATCCCCTGGAGCTGG
GGTTACAGTTGGTTGTAACTTCCCAACATGGGAACTGAACTCAGATCCTCTGCAAGAGCAGAACACACTC
TGAACTGTTGAGCCATCTCTCTAGCCACTGACTCTGAAAATAGATTTAATGACATGTTAAGGCTATTACA
ATCTGGAAAAGGAAATTGTAAGGCTAGGATGGATCCCAGAAGATACTAAACTCAAAACATTAATCATACT
CTTTTTGGTTTTCTTTCTTTTTTTGGGGGGGGGGGGGGTTCGAGACAGGGTTTCTCTGTATAGCCCTGGC
TGTCCTGGAGCTCACTTTGTAGACCAGGCTGGCCTCGAACTTAGAAATCCGCCTGCCTCTGCCTCCCGAG
TGCTGGGATTAAAGGCGTGCGCCACCACGCCTGGCTTTTTCTTTTGTTTTGATAAGGTGGCAGTATCTCT
CTAGCCCATGTAGCCCAGGCTGGCCCTGAACTGTAAGCTTTTTCCATTCCAGCCTCCTGGCTTATACAAA
CTAAGTGGCTTTTCTTTCCTGTCCACCTGCTTGGTTTTGGCTTTTCAAGATAGAGTCTCAGGTACTGCAG
GTTGACCTTGAACTTGCTATATAGCCAAACAAAGAACCTTGAATTTGCAAGTGTTGGGATTACAGGTATG
TGTCACCATAACTAGCAAGGCTTTCTTTCCTCTTACCTTCTTTTCACCTTATTTATTTATTTATTTATTT
ATTTATTTATTTATTTATTTTTGGTCATCAATAAGAAATTCCCTTGTGTATGTGTGTTTGTTTGTTTGTT
TCTTTTTTAGCATATAAATGCTAGCATTGTCCAAAAAACTGTAAGAGATTGATTTCTAATTGTCTTGAAG
TTGAGCTGCTGGGAGTTGCCCAAAGAAAGGCTTGGCTCTATTGGGCTTTACCTCCCGTGTTTGGATTGGA
AATCGAGTACAAATGAAAATGGAAAGCCCGCCAGCACCTGACTCTGCCCCATTTTTCTACGCAGAATCAT
GCCTTTTGACCCCACAGGGACGAATCTGACATTCAGAATTACTGATGATAGCAGGACAAAGGGGTTTATT
GTTTGCTTCGCTTTGTTATATTTCTCTCATAGTAGATTCTTTTTGGGGGGTCAGGGTTTGAGACAGGGTT
TCTCTGTGTAGCCCTGGCTATCCTGGAACTCCCTCAGTTGATCAGGCTAGCCTCCAACTCAGAGATCTGC
CTGCTACTGCCTCCTGACTCTTGGTTCCTGGCACTAACAGTGTGCTCTACCATGCTCGGCTCTTATTGTA
GATTCTTAAGCCTTCAGGCATGCTAGCTGGAGTTCTTTGGCATAACTGTTAGTTTGCATGGCTAGCACAC
AGCTGGTATCCTCAGAAAGGCCTGGCTTGAGTCCTGTGAAGCACCAGTAGCTGTACTCACGTCCTGGGTC
TGGCCCAGATGCTAACCAGAGTCTGGATCAGAAGTGCAGCAGAACAGAGTGCCACAGTACCACGTGGCAC
AGAGAGGTCCCCTCACCCCTGCAGTAGTGCAAGCAGCTATGGAGCTAATAGTTTACTAGGTGCTTTTCTG
CTGGTGGGTATGGAGCTGTCCACTTCATAGGAACAACCGTATAGAGACCTTCCTCACTATCTCCCTCGTA
TAACCCAGCCAAAAGCTAGAGGCTGCATGTCTGTTCTTAAATCACACTAAATTGATACCTCAAGAACTAT
TTTTAGTGTAAGTCCTCATGTCATTGATTTTTAAATTCTTTCCTAAAATTTGTATGTTGTAGTTTTAAAT
GCTTTCTTCATGTTTGCTGTCTAACATTGTTAAGAAAAGTAAGGAAAATTTGTATTTGTATTAATTTGTC
AGAGTGTCTCAAGAACAGCTTTTTAAAAAATTTTTTTCCATTGTCACTTGCAAATATTTTAGAGTTTATA
GTATAATTACACTTTTTTCCAAGTATTTTGGATAAATAGAATATTCAGCATATAAAGATAAAACTAAGTT
AATAAGCCGGGTGTGATGGTGCACACCTTTAATCCCAGCACTCGGGAGGCAGAGGCAGGCGGATTTCTGA
GTTCGAGGCCAGCCTGATCTATTTACAAAGTGAGTTCCAGGACAGCCAGGGCTACACAGAGAAACCCTGT
CTTGAAAAACCAAAAAAAAAAAAAAAAAAAAAAAACCTAAGTTAACAAACTTCATTCAGTCATAAAACTG
TTAAATCGAAAACACTTTTACTATGGTATATGACATTTTTCTTTTTAAGATTTCTTTTTAAAATTTAGGT
GCATGCCCCTGTTCTTGTTTGTGCACTGTGTGTGTGCAGGTACCCAAAGGACCAGAACATGACGCTGGGC
CCCCTGGACTGCAGTTACAAGCAGTAGTGAGCTGCCTGATGTGGGTGCTGGGAACCTAACCTGGGTCCTC
TACAAGAGCAACAAGAACTCTTACCATGACCCCAATGTGACATTTAAAAAAAAAATTCTTCCTTGTGACC
TAAGATTGTTTTTCCATCTAGGGATTTCAAAGTATATTATATTTTCTTTCTGTGGATTCCTGCCCCATTT
TAAAAAACAAAAAACAGTATCTAACCAGGACCCATTTCTTTTTTATAAACCATGACAAGCACCTGTGTTT
TGAGGCTCCCTGATGTTTGATTTCTTTGCTCTTTGCCAAATTTTGCTCCTTCCTTTCAGGTTCTGTTTTC
CTCAGAGTATCTGCTAGAAATGTTTCTAGAGGATGAGTGTGTCGTGAAGTTGTTGGCTGTCTTTCACGGA
AAGCAACCTTTATTTGGCCTCTTTTTTTTTCATGGTCCTTGGTCTTAGTTGCATGTGAGGTCCTGGGTAG
CAGCTCCGAGTGGTCCCTGGAGACCGCCGACTCCACATCTGCTCTTACAGAGTGCCAGTGTAACTGTCAT
TTCCCTCTGAGGCATCTGTCTTTGGGTTTCTGGTATTTGGAATTCAAGTGTAGCACCCTACACAGAAGGG
TGTTATCATCTGTTAAGTGCAGGCCCTGGGGGGCAGCTGGACCAGTCACCCTGTGCACAGCTTGCCACTC
AAAGGTAATCTAATGGAGACCGTAGCTGGAACGGAATTCCAAAGAAACTGAAACACCCGGTCTCCATGCC
TCGCCGCTACAAGGCACAAGTGGATATTTACAGGTAGAAGAGCCTCAAGAGAAAGATTAGCCTAAACATC
AAGCCAGATTCATCCGACCCTGTGCCTGCTGCCTCATTTGTCACAGCATGGGCCGTTCTCTCTGCCCTAG
CTCTTGCGTAGTTTATCTGACACATCCTTCTCTCTCGAGTGTTGCCCCTCAGAGTGTTAGCCCCTCAGAT
CCGCTGCCTAGTTTGCCGTGTTCTTTTCAGCTGAGAGAACCTGTTTAGTTTCCACTGTGTTGCTAATTTT
ATTTACTATTTTTATAATGTGTTCTTTTTTAAAATACCCTTTAAAACTCCACCCATTTCTTTTTACTTGC
TTCTAGTATTATTCTCTAAAATAATTTTAAACATACATTATGATTTTTTTTGGAGGCAGAATTTCATGTA
GCTCAGCCTGACCTTGAACTCACTGTTCAACCAAAAATGAACTTGAGTTTTTGATCTCCTGCCTCTACCT
CCTCAGTGCTAGGATTACAGGTGATCACTTTGGTTCAATGCTGGGTGGGATGGGATGGATACATATACAT
ACACATATACATATACATATACATCCATATGTATATATATATATTCTACTATGCAAGCACTCTGCTAACT
GAGCTACATGTCCAGCCCATAAACATGTGCTTTGTAGATTGCTCTAAAGTCCAGTTTTGGGGGTGCTGGT
TAATCTCTTTTATAATTGCATATTTTAATTTCTGTTTTGATCCAGAACTCCTCCTCTAAGAGAATGGGTG
CCATCGATGAGACTGGTAGTCTTTGTTAGTGTAGGGCGTGAGCGGCCAGCTCCACACCGCTCTCCTCATT
TGTTAGCACCATCGGGGGTTGATGATGAATATGGTCCAAAATTGTTTTCTTAACTTTTTTTTTCCTTAAA
GTTTTATTTATTTTATTTGTATGACTACACTGTTGCTGTCTTCAGACACACTAGAAGAAGGCATCGGATC
CCATTACAGATGGTTGGGAGCCACCATGTAGTTGCTGGGAATTGAACTCAGGACTCTGGAAGAACTCTGA
GCCATCTCTCCAGCCCCCAATTTTTATATAATCTCTTGCTTAGGGCTCTTATTCCAAGGGAGTGTCCACT
TATGCCCCATAGCTACAGCCATTGGAGACCGGGTGTTTTAACGTCTTTAAAACTATTTTCTAACCACAGC
CTCCATTTCACTGCCTTAGAGTTGGAGGCTTTGGGTAAGGTTTGGTCCAGTTGTCCAGACCCTGTTAGCC
ACTGCACATTCTCTGCCCCAGGACCTGATGTCTGAGGCTCCCGTGCAGTACCTCTGGTACTTGATGGTGT
CACTGCTGTGTCTGAGGTCAGGTCCCACATAGTCACAATCCTTGAGTTCCCTCAAGAGTGTTCTGTCTTC
CACTTAGGCCCCAGTAGTCTTCACAGCACAAGCTTCACAGGGCAGTGTCCTCATCCCTTCCCCTCTGGCC
TGGCCTCCCCCGTTCACCCTGCCTTTGTGCTCTTCTCGAGATTTTTACATCTTTCTTTTGTCTCCCTCTT
TGTTCTCCCACTGTGGGTACATTCTGCCCTGTGTTCATCGCCTAGATCTGTCAGATGGTTTTCTGACCCT
TGAAAGCCCAAGTCATTTCTTGTGTCCTTTTAGCTTCTTCATGTTTTGTTTTTGTTTTCTTTTTTTACTT
AAATCAAAGCATGCATGCGTGCATGTGTGCTGTGGTGGAGGGCTGAGTGGAGAAACCAGGGGTCTGTCTT
GTGGCTCCTGCCTGCCGTGTGTGCTCATGTGTCCTTCCCTTTAACTGTCCCTGCAGGGCTGTGGTGTTTG
TGGTGGACAGTGCGGCGTTCCAGCGGGAGGTGAAAGATGTGGCCGAATTTCTGTACCAGGTCCTCATCGA
CAGCATGGCTCTGAAGAACTCACCGTCCTTATTAATAGCCTGCAATAAGCAAGGTGCTTCCTGGCAAAGG
CAGCCAACCGTTGAGGATTGCTGTTGGGAATTAGTTCATTTACCCGCCGTTGTTATTTCTGAGCATGGGG
AAAGGGCTGGAATTAAGCATTAATTAAGGGATTAAATCTGAAAATTGTTTCTATTACTTTCCTGGTCTTT
TACCAGGTTGTCCCAAGGACTGGCAGCTGGGGGCTCTGTTGATATTATATCTTTTTCATGATACAGTGCT
TTTTAAAGATAAATTATCTTACTTTTCCAAATGTTAATTTGTGAGACCGAGTACTATCGGTTCTGGGGGG
ATTATTGGGCCTCTCCTTCCAGACAGGCTGCGCCACTGTTCCCTCTTACCCCAGCGCTCACAGTCAGGTT
GGCTGGGCATCAGCTCTGTGGCCAGGCGGAGTAACGTCAGGAGTTGGTCCTCGGGAACTCCTTCACGGCT
ACATTTCCTAATACCTGATGAATGGTGCAGCTCTTAGCATCTTTTGTAAAAGTATTTTTGTTTTGTGTGT
ATGTGGGGCGGGGGGGGGGGGGGGGTGTGCAGGGCTGAACCTCAGGCATACCCGGTGCCTGAGAAGATTA
GAAGAGGGCTTTGGATCTGCTGGGACAGTTGTGAGTTGGCCATGCAGGTTCTGGTACTTGAACCCTGGTC
CTCTGGAAGAGCAGCCAGTGCTGTAACCCCTGGGCCATCTCTAGCCCTAGCCATCATCTTTTCTTAAGGG
TTAATGTGGTGTTGTGTTGAAAGTTCTCCTCAGATGTCAGTCATGGTTCACGTATGCTTGCTTTCTTTCA
ATGGACAGGACAGGTTACATATTGAAGGGCTTGTAAGGTGTGGGAAACTTAAGCTTGGTTAGTTTTAAGG
TCATTAGCACTAAAACAATATTTTGTTGTATTTTATGTTTTAAAACATTTTTTAAAAACCTGCAAATACA
GCATTTACTGTGACAGAAAGGAATAATGATAAAAAGTGTTTCAACAAAGGAATGTGGGGTGAACTAGTTG
GTATAATTGGGAATTTTCTTTCTGTGCTGGAAGTCCAGGGAGGAGACAGATCCTGGTTTAAACTAGAATA
ATGCTCTTCTTTTCCTTCCCAGATATCGCAATGGCAAAATCAGCGAAGTTGATCCAGCAGCAGCTTGAGA
AGGAGCTGTAAGTGGGGCTGTGCTCCGGGTGGCGTCCTGACCGTCGGAGTCCATTCGCAGCTTAGGATCG
AGTGATCAGAGTTGTAGCTGGGGTAGTATGGAATATGTGTTAAAGGGATTATTAGAATTATAAAAGACGT
GGGTGTTTATAGCCATTTAAGGTTTTTCACAAATTAGGGAAAGGTGTGTGGCACCATGCCCTGCTTGTAA
ATTTAGTCAGATGTAGTATTTATGAAATGTCCCCAGCAGGAAGGCAGCAAGCATGCTGCCTGTGTGCTCT
ACAGGGCCAGCGGCTGGCAGCACAGTGACAGTTTGGGAAGGTTTTCCTGCTTACAGATACAGGACAGATG
AGACGGAAGTCTGTGGATGTGTGAGCTTCATAGCCAAGGCTCCCTATCTACCCTTTCTGAAGTTTCAAAA
CCATGAGTTTTATAATACAGTGGGGCCATTCAGGAGAGCTTAGAAGTAGGTAGAGGCTTCAAAGAGCTGG
TGTGACCTGGGTCTCCTAACCCACTGGTGTCTGTCTGAAACAGTGATCCATGGACATAAAGGACAAGCCA
GCAGTGTCAATACCGACTGGTGCTATCCCTTTGATTTAGGAGCTCATAGGATTTTGGGCTTTGTTGCGTC
TGACTTTTCTCCCTTTGCCCCCAGCAACACCTTACGAGTCACCCGGTCCGCGGCTCCCAGCACACTGGAC
AGTTCCAGCACCGCACCTGCTCAGTTGGGGAAGAAAGGCAAAGAATTTGAATTTTCCCAGTTGCCACTCA
AAGTGGAGTTCCTGGAGTGTAGTGCCAAGGGTGGACGAGGGGACACTGGCTCTGCTGACATCCAGGACTT
GGAGAAGTGGCTGGCCAAGATTGCCTGAGGAGCATCGCTGGCCACAAGCGTTGGTCATGTGAGACTGACC
CTGCAGTTTGAGAAAGGGCGGTGGTGGTGTGGACTGATAAAGAGGGGGCATGTCGTTGAAGCATTTCTTG
AGTTCTTGAAACAATTGTACCACCCCCACCCCACCCCCGTCAGCTCCTTTCTCAGCTAGTCCTTTCTTAA
TTCCCATATTCTTTCTCCCTTTGCTAGGTGTTAGGTGTAGCATCATGTTAGTGTGAGACATAACAGAGAG
GGCATCGCTTTGAGAGTAGGGTCGCATCTGAGAAGTCCTTGTCTCCTGCCTCTGTGTTGGTCCCTTTTCC
TGGGACCAGATCTGCCTGCTTGTGGAATAAGTGAACTTGCAGTTGCCCCTGGTGTAGGCATAATACAGTG
TCAGTTCTTAGTGGAGTCTGGGGTGTGAAGGTCTACAGTGAAGACAGCTTCAAAGCCACAGGCACAGCTG
ACAGTGTGTCCTTCACGGGCTCAGTGACCATCCCACACCACCCTAGCTGACAGTGTGTCCTTCACGGGCT
CGGTGACCATCCCACACCACCCTAGCTGACAGTGTGTCCTTCACGGGCTCGGTGACCATCCCACACCACC
CTAGCTGACAGTGTGTCCTTCACAGGCTCGGTGACCATCCCACACCACCCTAGCTGACAGTGTGTCCTTC
ATGGGCTCGGTGACCATCCCACACCACCCTAGCTGACAGTGTGTCCTTCACGGGCTCGGTGACCATCCCC
ACACCACCCTAGGTTCAAAAGCTGCCGCAACCCCGAAGAGTCACCCTCACAAGGTGGCTCTAGGAGCCAA
ATCCCCAGACACTCCTAGGTTAGTGTTCTAGAGGCTCCACTGGGGAGAGCAGGGAGACAGCACACTGCTG
CCTCGGGCGTGCGTGAGCCCAGGCTGTCCCCTGTGCTACTTGATCAGCTCCTGTTGTGTCACCCCAGCTA
GAGTGCTATGGAACAATACAAACTGAAGATGTCCACTGTGCATTTTAAATACATATTTATTCATGATGCT
TTAAAAAATGTTACCTTTTTAAAGTGATGCTGTCTTCAACATTTGTGTTCTCATTGCTTTTATAAATGAC
TGGTATCTTGATAAATGACTATACTGAAAGCATTGAGTCAAATGATGAGTACAAGTGGGAACCTGAGCTA
GAATTGAACCCCAGCTCCAGGTTACTGAGCCTACAGATCGCTGCATATATGAGACTCTTGGTCTCGTCTC
ACACTCAGAGTCAGGCTTCACTAAAGCCACACTAAAGAGACAGGCAGAGCATTGACTCAGGTGCAGTGCT
CAGGGAATATGTGTGGCGCTCTGGGCTGGCCTGGACCAGTGAGAGGGACAGGATAAACACACAGGTGTTC
TCTGTGCAGACCAGCCCAAGCCTCTGCCTGCTGACACATGGCCTCCCTGTGGGTCACCTGGGCTGATAAT
AGAGTCCCAGACCATGATTCCCATCAAGTACTCAGCTCCCCACTCCTAAAATATCTACTGGCCTTGCTTA
TGGTGTATGAGACAGATACAGCACAGCACAGGACAGATAAGCCATGAGATGACTCAGTTCTCACTGGGAG
GCAGTATTGCTGCTAGGATTCTTACTGCCCTTTGCACACTGCTGTTCATGTTGGTCACGGACAAATGGGA
AGGGCACGATGAAGGACGTTCACATCACAGTAGGTGCTCATCAAAAAAACCTTTCAGTTAGAAAATGTGT
TCGCTGGCCTCCCTTTTGTCTGCATAATAAAGTGATTTTACTTAGTGGGAGAATCAGTACATCAAAAAAG
TGACAAGGATGGAGAGCAAGGATAAATGTTATTTATTTATTTATAGCCTGTGTCCTGAGCAAAAGGTAAG
CTGTGCAGGGCTGGGAACCAGGTTAGTAGAATGCCTACCTGGAACGCATGGGTCTGGCTTTGCTGTGGGC
TGCTGTAAAAGCCAGCTCTAAACAGGCAGCCGCAGCAAAGATTCTATGTCTGGACTCTTCCATGGAGCCA
CGGCAGAAGCAGCTTCTCACTGCTAGGTGCCTTGTGGTCTCTCGGGAGGCCTTGGCCCCTGCCACTGCTT
CCCAGGGCCTCCAGCACCTCCTGACGTGAGCTGTTCTATCAGAGGGGAGGGGAGGCAGTGGCCCAGCCAT
TGAGCTATTTCAGGCACAATAAAATATAAGGCT

An exemplary DNA sequence of the Srprb gene in humans is provided below.

(SEQ ID NO: 22)

GCTGTACCGGGGACCACGCGTCTCATCCATGGCTTCCGCGGACTCGCGCC
GGCTGGCAGATGGCGGCGGTGCCGGGGGCACCTTCCAGCCCTACCTAGAC
ACCTTGCGGCAGGAGCTGCAGCAGACGGACCCAACGCTGTTGTCAGTAGT
GGTGGCGGTTCTTGCGGTGCTGCTGACGCTAGTCTTCTGGAAGTTAATCC
GGAGCAGAAGGAGCAGTCAGAGAGCTGTTCTTCTTGTTGGCCTTTGTGAT
TCCGGGAAAACGTTGCTCTTTGTCAGGTTGTTAACAGGCCTTTATAGAGA
CACTCAGACGTCCATTACTGACAGCTGTGCTGTATACAGAGTCAACAATA
ACAGGGGCAATAGTCTGACCTTGATTGACCTTCCCGGCCATGAGAGTTTG
AGGCTTCAGTTCTTAGAGCGGTTTAAGTCTTCAGCCAGGGCTATTGTGTT
TGTTGTGGATAGTGCAGCATTCCAGCGAGAGGTGAAAGATGTGGCTGAGT
TTCTGTATCAAGTCCTCATTGACAGTATGGGTCTGAAGAATACACCATCA
TTCTTAATAGCCTGCAATAAGCAAGATATTGCAATGGCAAAATCAGCAAA
GTTAATTCAACAGCAGCTGGAGAAAGAACTCAACACCTTACGAGTTACCC
GTTCTGCTGCCCCCAGCACACTGGACAGTTCCAGCACTGCCCCTGCTCAG
CTGGGGAAGAAAGGCAAAGAGTTTGAATTCTCACAGTTGCCCCTCAAAGT
GGAGTTCCTGGAGTGCAGTGCCAAGGGTGGAAGAGGGGACGTGGGCTCTG
CTGACATCCAGGACTTGGAGAAATGGCTGGCTAAAATTGCCTGAGAGGCA
GCTCTAAAGCACAAGACCTGGATGTGTGACACACAGTTTTGGAAAAAGGT
CTGTGGTAGTCTGGAGTTGATGAGGAAGGGGTACAAGATGTGGTTAGAAA
CATTTCTTTGTTCTGGAAACAAAGTACTGTTGAAACCAGCTTGGAATTTT
TTTTTTTTTTTTTTTTTAAGTTCAGTTCTCCCTTATGGCTGCCTTTCAAA
CAAGTACCTTTTATCTGATGCCTGTATCTTCCCTTTGTTAAGGTGTAACT
TGATGTAGGGTCAAGGTTTTTGTGACAACAGGCAGACTCCACACAGAGAG
GATATGATGAGAATATGGCCATCACCTGAAAAGTTTTCTTATCTTCTGTG
CTTTTGGTCCCTGGAAACAAATCCGCCTATGTATGAAGCTAGTTGATTTC
CAGTTGCACTATTTCCAGTTGCCTCTGAAGTTCACAGGCAATACATTGTC
TAGTCCTTTGCGAATTTCTCTGATTTGTGGGCACAGTTATGAAGTTTCCC
CACATGTGAAGACAGGTACAAAATAGCAGAGCCAAGCAGACAGTGGGTCT
ATTCTTCATTAGCTCAGTGACTTGTCCACACTCGTCTTAGCACTTACGTT
TCAAAAGCTTGTCACAAACCCTTGGAGTCATTCCCAGATAATAGAACTGG
AAATGATAAATCCCCTAATGCCAAGGGTCTAGTGTGTTCTTAGTGGTTAT
ACTGGGAAGTGTGTGGAGATTTAGGTGCTGCTCTGCTGCTCTGGATGGCT
GAAGGCTCCTGGGCCATCTTCATGTGCTGCTTGAAGAGCTCCTATTTTGT
ACTCCTGGCTAGAATGCTGTGGAACAAATACAAAGTGAAAAAAGTTCTCT
GTAGATTTCTGAAGTGCATATTCATTGATGCCAAGAAAAAAAAAAAGTTG
CCTTTTTGAAAAAAAAAAAAAAAAAAAAAAAAAAAAAAAAAAAAAAAAAA
AAAAAAAAAAAAAAAAAAAAAAAAAAAAAA

Trf, or transferrin, is an iron-binding blood plasma glycoprotein that regulates the level of free iron in biological fluids. The Trf mRNA sequence in Mus musculus is provided in GenBank Accession No. NM 133977.2 (GI:118129942), incorporated herein by reference. The Trf amino acid sequence in Mus musculus is provided in GenBank Accession No. AAH92046.1 (GI:62027488), incorporated herein by reference. The Trf mRNA sequence in Homo sapiens is provided in GenBank Accession No. NM_001063.3 (GI:257196241), incorporated herein by reference. The Trf amino acid sequence in Homo sapiens is provided in GenBank Accession No. AAB22049.1 (GI:248648), incorporated herein by reference.

An exemplary DNA sequence of the Trf gene in Mus musculus strain C57BL/6J chromosome 9, GRCm38.p1 C57BL/6J is provided below.

NC_000075.6 (GI: 372099101: c103230286-103208876):
(SEQ ID NO: 9)
GCCACGGGCAGGCGGCGCAGAAGCGGGTCGGTCTGTACTCCCCGCTCCTCGCCACACACACACCGAGAGG
ATGAGGCTCACCGTGGGTGCCCTGCTGGCCTGCGCTGCCCTGGGTGAGTGTGCGTGCAGGGGATGACTGG
CTGGCTATGTATGCAACCCTTAGGAGCTAAAGCTGGAAGTCCCAGCTTCCAGCCCCTTGCTTCTTCTCTG
TGGTATCTTCATCTTCCATAGCGTAGGAACAACGTGGATCCACATGCATCTGGGGCTCTGATTCAGTCTC
TACCTTTCACTATCCTCATCACGCTGTCTCCCCTATGTCCAGTAATTTGAAGATATGGTATACGCGATGT
TAGAAAACTTTTAACCAGTTTACCCAGGCGCTATCCCGGGGCCTCAGTATAGTCCAAACAGCATGGTGTG
GTAGTCACAACTTCCTGAGATCTAATCCTGCTATCTCTCCTTTGTCTGATGCCTCCTTGGATGCCTTTGG
CGCTGGTGGCTTAGACTAGGGTGTGGGATTTGAAGCCAGAATTCTGGGGTTCAAATCATACCTGTATTTC
TCCTGCGACGGGTCTTGCTTGTTACCGCACATGCCTGGGAGGGTCAGGTGAGGGTGAGGTCACAGGTCCC
AGTCCTGCATCCTGCTGACTCCTCACCAGAGGGGGGGGGGGTCTCAAAGACCCAAATGCCTGCTCCAGTT
GGACTGTAGTTAGCATTTGTCACAGTGTTGGGGCTGTCTAGTTTGAGTGATTTGTCATCCCTAACAACTG
TCAGCCTGAAACAGGCTTCCGTGCTTGTTAGCTCAGGACACCTTGTGTGGCTATGCACAGTGACACCGGA
CCAAGGTCAAAATTTACAAGTTAGACGAACTAGTTTGAAGAGGCTAGAGAGGTCGTCTATCCCTCTGGCC
ACGGTGAACTAAAGGGAAGGAGCTGGGGACGATCAGTCTCCCTGTTGTCCTTCCTCATCCATCCTGAGGT
CCATTGGCCTCACCCACCCCGATCCATGCTTAGTGATTTGTGGGCTTGCTGAGTCAGTAAGTCAGCCCTG
CTGAATTGCCCGAAAGGGAAGGGATGTGACAAGAGCTAGGAGTGCCAGGTCCCACAGACGGCATCTCTCC
ATCGTGTGGATTTCAGTGGTGACCCAGCTGGGGAGATGGTACAACTGGGCAGATGCTTAGCATGCAAACA
CCAGGACCTAAATTCAGAACCCCAGCAACCATGTAAAAAGCTGGGTACCGCAGTGCCCTGGGGAAGCAGA
CACAGGAAGATCCCAGGGAACCCACAGTGCCCTGGGGAAGCAAACACAGGAAGATCCCAGGGACTTTCTG
GCCAGACAATCTAGTGGAACCAGCAAGGCTCAGATTCAGTGAAAGACACTGTCTCCAAAAAAATCAGGCA
GAGAGTAATAGCGGGAGACACCGCACGTTGCCCTTTGTCCCCCACCCCCACCCCCCATGTGTTCCCATCT
GCACATGCACACACCACATGGGAGCCCGATTATATAAGCACATACACACATCGTGATACACGTAATTGTC
TTCAGCTGATTCTCTAAATGGCGGCCCAAAGGGGGTAAAGCATCCAGGGAAGGTTTGGTAGAAACAGAAG
CCTGCTGCATAGAAGGGAGTTCCTAGCCTTTTCTCAACAGAAGACAGCTGAAGAGTATCACAGGTGAACT
CGTGATCAAACTGTCGCTCAGAGAGACGCAAAGCTTCTAAGATCGTTATCTTCCCCTGGGGCCTGGCCAT
CAGACCTAGTGTCTGGGCTGCTAACAAACATGATGACGTAGGACAAACCTGCTTCCTCCCCCAAGGCCAG
CTTTCTCATTTCTAAAAGACGGTATGCCTTTCCTAGAGTTGGTGTCTAGAGCCTTGTCTGAAGGTGGCTG
AGGATGGTGTTTGCCCACTGAAGTTCACATTTTGATGTGGACCTTCAGCCAAGGCAAAAGAAGAGCAGGT
TGTCACCTTCAGCTACCCCAGCATCTCTCTAAGCAGCCCAGGGTTAGGTGGGAACATGGCAGACCGTTGG
GGCGAACGGATTCATCTGTTCCCAGGATCGGAGTGCAGCAGAGCCCTGTGCTCTGTCATGACTGCCACCA
GGGATGAGAGGAGGGTGGAACAGAGGAGAGGTGGCCATTAAGACCCTTCGTAAAGTGGTGCGTTCTCTCC
CCAGGCACGGGGAGCGGGTCCTTTATGCCTCCGCCTTCTCTCCTGCAGGGCTGTGTCTGGCTGTCCCTGA
CAAAACGGTCAAATGGTGCGCAGTGTCAGAGCACGAGAATACCAAGTGCATCAGCTTCCGTGACCACATG
AAGACCGTCCTTCCGCCTGATGGCCCCCGGCTTGCCTGTGTGAAGAAAACCTCCTATCCGGATTGCATCA
AGGCCATTTCTGTAAGTCCTGCTGCCGGGAACGGTAGCCCCTATACGCCTCTGGTTTGACTCACTTGTGG
AAAAAAAAAAATTGAACTGGGGGGTGGGGACTGTCAGGTGTCCAAGTAGACAATTATACCAAGAGTGGCA
GGAAGAGGATCATTCCAAGGCTTGAATTCTCCTGTCCATCCGGGTGTGGCCGCACACGTAGGTGGCCATG
GCATGCTAGCTCCTACTGTTTCAGGGGCCCAGGACTATAGGGAAAAGCAGTAAGCTTTTCAGTGTTAAGA
CTAGCACCTTCTGGGCTAGTGAGATGGGCTTCTGAAGGTCCTGAGTTCAAATCCCAGCAACCACATGGTG
GCTCACAACCATCCGTAATGAGATCTGACACCCTTTTCTGGTGCATCTGAAGACAGCTACAGTGTGCTTA
GATATAATAATAAATAAATCTTTAAAAAAAAAAAAAAACAAAGACAGCACCTTCAGGGTTTTCACCTCCA
TGACAGCACCCACAGAGGAGCTTACAGGGACTCTCAAAACACGAGGGTCTCTGAATGACAAACCCCACTG
TGTGGCCGAGCATGGAAACAGATATAAGAACCCAGCCACCCACAGACCCTGGGTGCTCCACTACAACCAT
GCCAGAGCCTCTGGTATAATAGCAGTGCCCACCTCTGAAGAGGGAGAGACCCTTCCCCCCTACCAAGCCT
GCCAGACCAAGCCTCGACCACTGCCGATGGGTAAATCCGCATTTTGTCCAGGTACTGGGCACATCATCAG
GGAAAAGTGAGGAAAAAAAAGACTCCATTTCTTCCTCTAGATGGAATCAACCCAAACCGGCAGGGCTGGA
TGTGCTTGTGCTTGGGATTTTCTAGCCCGAGACCCATGGAGCGCTGCCAGGTCCTTCCCACTTGTTCTGG
TGTAGGGAAGCCTTCCTTGCAATAAGTCTTTGGGACTCGCTCTCTTCTAGGCAAGTGAAGCCGATGCTAT
GACCTTGGATGGGGGTTGGGTGTACGATGCCGGCCTGACTCCGAACAACCTGAAGCCCGTGGCGGCGGAG
TTTTATGGATCAGTGGAACGTGAGTTCTCCCAGAATACCCAGGAGGGGGACCGTGGTTTAGTAGATGTTA
AATCTCATTCTGTAGGCAGGTAGCCGACATTAGAAACGCTAGGAAGCCTTTTCTTTGGGGAGTCCATGGG
GAATGAGATCTTCTAGCCCTCCGGGCACACATGGAGACATCTTGAGGACTTTGGGCTGGTTGGAGGCTGG
TATGGACCTTCTGAGTCAGGCATCCTCAATGGAGAGCAGGCTGGGGTCCAGCTTGGCCTTCACTGGATCT
GGGGAAGTGTCCAAGCTCCCCAGAGCCTCAGGCGCCATGGACAAGAGAAAACCCACCCCTATAGTGCCCG
GAGGGAGCTTAGCAGTCCAAGAAGCTCTTCTGAGATAGCTCTGGTGGTCCCTGTCACCATATTAGAGGAT
GTAAAATACTTTGAGGGAGACAGAGAATGCCTCTGTTGTGGCTCTCACATGTTCCTGGTGCCTTGGCTTG
GTAAGGAGAATGAGGCGCTCATGACCCATGTGCCGATGCCTCTTGAGCCTTTGTTTTCCCGGATGGACCA
TCTGTCTTTAAGTCTACAGAGGTTTCTCTGGCTTCTTACCACCCAGCTCTGCCTAAGTTACCCCTGGGTC
TCTCTCTCTCCTTCATCAGGACAGAGCACTGACAAGCAGTGCTTCTCTGGGCCATGTCAAAGTCCTCCCT
CCACATTGTCCCCTTTCAGATCCACAGACCTACTACTACGCTGTGGCTGTGGTAAAGAAGGGAACAGACT
TCCAGCTGAACCAGCTCGAAGGCAAGAAGTCCTGCCACACAGGCCTGGGAAGGTCTGCAGGCTGGGTCAT
CCCCATTGGCTTGCTCTTCTGTAAGCTGTCGGAGCCCCGCAGTCCTCTTGAGAAAGGTAAGCCGCCATTT
CTATTCTGAATATCCTGTACTGCTGGGGCCACACAGTCCTGGTTAGTCAGGCAGGTAGGCTGAACTCCAA
GGAAGTGGAGGAAGAGGGAAACAATTAGAAGTGGCAACTTGAACCTTCTTGTTGTTGTGAGCTCCCAACA
CCCCTGCAGAGGCTGACCTGAGGAGGAATGAAAGGGCAGGTGGGTCCACAGTGGATTTCCATATTCACTT
CAGTCCCTCACACACCCTCAACCCCCATCACCCCCACCACCACACACACCTCTGCTCCGTTAAAGGATGC
TTTGGTTCCACCTGCTGGTCATAAGGTTGTCCTGCACCTCTCTAGGCTGTACTACAGCCCCCAGAGGCAC
CCTTTGAAAGATCTGGAGCCTGGCAGTGGTGGCTCACACCTTTAATCCCAGCACTTGGGAGGCAGAGGCA
GGTGGCTTTCTGAGTTCGAGGCCAGCCTGGTCTACGGAGTGAGTTCCTGGACAGCCAGGGCTACACAGAG
AAACCCTGTCTTGAAAAAAACAAAAACAAAAAAAAACCTGGAAACAGAGTGGGCAAAGTCTAGGGCAAGC
CTTGTTTAGCAAGAAGTACTGCTTGTGTCCCCGGGAGGGTAACTGAAGAGCAGGGGTTAACTTATTCCTG
TCTAGAAACCCTGCTTAGTACATGAAAGGCTGGGCAGGGACTGTGCTGGGCAGGGACTGTGCCTCTGCCT
TCCTTCACCAGCCCAGGCTGGGTGATGGCATTATGCTTATTATGCCTTGTCCATTTCTTTGTATCGAGCA
GCTGTGTCCAGTTTCTTCTCGGGCAGTTGTGTCCCCTGTGCAGATCCAGTGGCCTTCCCCAAACTGTGTC
AACTGTGCCCAGGCTGTGGCTGCTCCTCCACTCAACCATTCTTTGGCTACGTAGGCGCATTCAAGTGAGT
GGAATTGACACTCTGTGGTGGCCAGTGCCAAAAGTCCTCAAATGGGGGCTCTTTGCTGAGCCAACCTACC
TAAAAGCATGCAGGTGGCTTAAGGTCCTCCCAGGAAGGCGGGGTTACAACCTGGGGCTCTCAAGGCCAAG
GTAACCTATGTCCCCTTCCATCTGATGTCAAAGTTCAAGCCCTGTTCTGGCAGTCCTCTCTCCTCAGAGC
TTCCGCATCTGGCTGGCGTCTCCTCTCAGTGGGCTGGGTAGCATCTGAAGGGACCTACATACCCCGAGTG
CCTCACAGTAGACCATGCTCAGGATACTAGACGCTGCCCCGCACCACCCCAGCAGTACATGTGGCTTTTT
AAATTATGTTTTTACATATAATATATTTTAATCATATATAAAACTGTCCAGTGGACATAAAATACTCTGA
GGGAGGCAGGGCATGTGAGCCTCTATTATGGCTCCATGAGCCTTGGGTTGGTAAGGACAATGAGGCGCTG
TGCCGGTCCCTCTTGGGCCTTTGTTCTCCTGGAATGATGCTACAGATGGACCATCTGTCTTTAAGTCTAC
AGAGGTTTCTCTGGCTTCTTACCACCCAGCTCTGCCTAAGTTACCCCTGGGTCTCTATCACCATACCAGA
GGACACTGCACCCCCATACCTTCTTACACACACACACCAACACTGAAGATTAAACCAAACCAAAAGTACA
CAGGGGAAAACAAAACAAAACATGAAGTCCTCTGTGTGTGTGTGTGTTGGCCAACTACTCCTGGGCATGA
GACTGGTCCTGAAATATGGTTGATATACTCCCTTAAAAAAACAAAAAACAAAAACAAAACAAAACAAAAC
AAAAACAACTGATTTTCGCCAGGCAGTGGTGGCACACGCCTTTAATCCTAGCACTTGGGAGGCAGAGGCA
GGCAGATTTCTGAGTTCAAGGCCAGCCTGGTCTACAAAGTGAGTTCCAGGACAGCTAGGCTACACAGAGA
AACACTATCTTGAAACAAACAAACAAACAAACAACCCTGATTTTCTGAAGCCAGCATCAATTGCAAGTAG
CCTCTTAGGAATAGGATTTTGTGTCATTTATGCCCAATTTCTTGCTCTCTCTTTTTTGTCTTCTGATACC
CTGAAATCCACATAGCCAGGGTAGAAATACTGTCATCTTCCATACTAAGGATCTGAAGATTTTTACATTA
TTTTCACCTCCTGTCAGCCACTTTGGGGTACGTGAGCACATCCCCCATCCCAGTGGTACTGTGGATGTCA
GCATCCCACCCTAGAGAGCTGTCTCCACTATGAGGGCCACTCAGCCTCAGGCCTTGTCTGAAAGGTGACA
GTGTGATCTGGACCAGGAGCAGGGTCCACCTTCTGGGACTCCTTGTTCATGGGCTTCTTTCCCACAGGTG
TCTGAAAGATGGCGGTGGGGATGTGGCCTTTGTCAAGCACACAACCATATTTGGTGAGTGATGGGGAAAG
ACTCTGGCCGGTTCCTTGTCATCCTTTTCATGGCTAGTGGCCAGACCTGAGCATGCAGGGTCTCTCTCCC
GCTGCTTGAGTCTCTATATATGCAGAGGAAGGTACTTTGGGGGTCTCTACCAAGATTCATGGGTCTCTTT
CCAACACTCAAACCACACAATGGCTCTCCCTCTAGGGAAGAAGTGTCACAGCCAAGGGTTGGAGGTTGGC
CTGGCTGACCTTGCCTGTCAGTAGTCTTGCTGAGCTTGGAGTCAGGGGATGGGGGCAACCATTGCTTCAC
TGGCCTACAGAAGCCTCTGTGTCTTTAACTGGTCATAGGCTCACCTTCCTGCATATTCCTTGACCCTCAG
AGATCCCTCTAGACCTTTCAGTTCATGCTTTCAATGCTAACAGGACTTCAAAAATGGGGAGGGGTTCAGC
CTGCCACAGGCTCACTGAACTTTCCCTCCTTAGAGGTCTTGCCGGAGAAGGCTGACAGGGACCAATATGA
ACTGCTCTGCCTTGACAATACCCGCAAGCCAGTGGATCAGTATGAGGATTGCTACCTGGCTCGGATCCCT
TCTCATGCTGTTGTGGCTCGAAAAAACAATGGCAAGGAAGACTTGATCTGGGAGATTCTCAAAGTGGCAC
AGGTATTTTACCGCCATCCTCTGTCCTGCTTACCGCTCTGTGCAGAGATCTCTTTGACATCTGGACACAC
TAAGACAGCCGGGTCACCAACCTCTCCAGCATCCTCATAGAGTAGCCAGTGTCTCCTCTGGTATCAACAT
CCACCACCTTTTTGACCAATGATACCCTTTCCATCTGAATTAGGCCCTCAAGGCCCAGAGAGAATCCCAG
AAGGAAAGAGAATCTCCGTCACACTCGCTCACGCGCGCGTGCACGCACACACACACACACACACACACAC
ACAGAGGCACACGCGCACACACCCAGACTGCAGAGGAACTGACTTCCCCTGCCTCTTGGCTGAGGTCGGT
TAACACAGTTTCCTCTGGTCAAGCCTACTCATGCCAGCCTCTGCACTAGATTCACTTGTTCTCAGCTAGT
TCTCACTTAACTACTCAGGCAGACAGGGAGAGGGAATGTGGGATGGTCCTTACAGGATCCCGGACAAAGA
GGCGCTGGGTGATGGAGAACATGCCCGAATAACTTCTCTCCTGGTACCTCCAAGTTTCCCACACTTTTAA
GATCAGTTTCCTGAAGTTTCTCTTGTTCTCTTCAGTTCTTGTTCTTCTTTTTAACCTCCCTAATTCCCTG
TTCACTCCACTGTTTTTATAGTTTTTTTGTTTTGTTTTGTTTTGTTTTTGTTGTTGTTGTTTTTCCTTCT
GGAAACAGGAACACTTTGGCAAAGGCAAATCAAAAGACTTCCAACTGTTCAGCTCTCCTCTTGGGAAAGA
CCTGCTGTTTAAAGATTCTGCCTTTGGGCTGTTAAGGGTCCCCCCAAGGATGGACTACAGGCTGTACCTT
GGCCATAACTATGTCACTGCCATTCGGAATCAGCAGGAAGGCGTGTGTAAGTATTCAGGGAAAACTAGGT
GACCCCAGGAATGTGGGGAACCTGGTAAGGTCAAGAAGCAGCCATGGTCTTTCTGGGGACTGACAGCTTC
TCCTGCTGTTCTCATTTCCATCCTGTTTGTGAGCCTAGGGCTAGACTGATGCTGGCTGGCATCAACCATG
AGAGGACCCAGAGGTCACCAAGTGCTTTAAAGGCTGGTCACATTTAAAATGACCTGGGTTCATACTAGTA
TCATTCCTCTGCAAAAATTAGGGTAGCCCCCTGCCCTTTATGAAGCCAATTAGACTATTAGAAAGTAAAT
AACTCCATGTTTCTGCAGACTGTATATCTCTTGCCAATATTGTGGTCAACATGGGCGTAATGAAAGCTGG
AGATGCAGGGAGTTTGATCTGGCCCTGGGTTTCACTGGAGGACTGGGGGTTTGCCTAATAATTTATTCAC
TCAGTAGGTATGTATTAGACAACCAGCCACAGACTCGGCTGGGTGCCTAACTTATAAACACATACTAGAC
TCTGCTGTTTGGGGAAGGAATTTCAGGTCAGGGAGAGGACAAATGTTTGAGAGATAGTCACACTACTGAA
CAGATAAATACAAAATGAAAGAATGAAAAACAGCAACAAAAAAAAAATCAAAGTCCTACAGTAGTGAAAA
CCAAAGTCAAACCTCTGAGGGTACTTGTACTAACATGTACAAACATACATACACACACACACACACACAT
TCATGCACTTACGCATGCATGCACGCACATACGCACACACGCACCCCACGTGATTAAAATTAAAGAAAAC
TAAATCTTACCTCGGAAGAAAAGCTGAACTTAAATGAAATCAACTACAGTTGGGCTGTGTCCCATCTGCT
TCCCTAGACGATTAGGAGTTCCTTGGTGGGATCTGTTTATTTAGCTCATCATTTCCATAAAGCACTTAGC
AGCATTGACATCTACCAGACAGCTGCTGCAGCCTTTCTTACCTGAGCCCCGGCTCTTCTTCTGGTACCAC
AGGCCCGGAGGGCTCGATCGACAACTCGCCAGTGAAGTGGTGTGCACTGAGTCACCTGGAGAGAACCAAG
TGTGACGAGTGGAGCATCATCAGTGAGGGAAAGATAGAGTGTGAGTCAGCAGAGACCACTGAGGACTGCA
TTGAAAAGATTGTGGTACGTGTCTCCTGCCTCTCAGGGTGGGTCCCCAACACTATGGCCTGCTGGCCCGC
TGAGTTAGCAAGTGTAACTCTTGCATGGAAATCCCCTTTCCAGACTCCTTGCTCTCATGGTTTGAGTTCA
GCTCTGAAGGATGCCCTTTACATTAGCGCAGTAGAAGGGTAAACTGAGCCATCTCCTGTCAAAGGACACT
GGACCATGACAAGAGTGCCTTTTGGGCTCAGGATGTGCCCTGGGGAGTGTGGAAAGGGCTCCGTTTTGAT
TTCGCAAGCAAGTACATGTTCACTTCATTGATCGAGATAGCATTTTTAAATTGCCTGTAGAGAATTTTCT
AAATCCTGAGCCCGAGAACGAAGAAAAACAGCACCCCTTGTCATCACCGTGACTCAGAGAAAACCCTCTT
CTTGAGAGTGTGCTGTGTGGGTTGCTTAGCACACCCCACCTTAGCACCTCATCGTGTCTTGTGAGCCTTT
CTTGTGTCTTTCGAACCCTGGCGTTTCTGCCTCAGAGAATACACTGAGGTTCTGCAGGGTGGCAGCCTTG
GAGAGGGCAAGGAAGCTCCAAGCTCTCACACCCCTGCCTCTGTTCTGTGCTCTATGGAGCCTCTCCAGTT
GCCTATTCTTTGAGTTGACTCTTTTGTGCTTTCTAAGATATCTATCAGAAAGCAAAACTGACTGCGAACC
CCATTCTTCTATCTACATTTTCAGATACTACATTCTTTTAGCTACAGACCATTTTCTTGTTTTGTCTGAG
TCAAGGCAAGAAAAATCAAACCAACTTCATACAAAGCTTGCTAACACAGAACCAGAGTTTATATAAAAAT
ACACTAACATCTGGGCGTGCCTGACTGATCATCAGGGGTCCGGTACCAACATCTACTGTTGCCCTTGGCT
TTATAACTTGGGCTGAGAGGGGCCTTCAAGAGGCCAGTCTAAAAGGGCAGGTGGCACAGGAAATAGCAAC
TCTTCATTTGCTGTGTCTTCCTGTGTCTTTGCAGAACGGAGAAGCGGACGCCATGACTTTGGATGGAGGA
CATGCCTACATTGCAGGCCAGTGTGGTCTAGTGCCTGTCATGGCAGAGTACTACGGTGAGTGAGGACAGT
GCCTCTGTGTTCTTTCTGAGCCATGGTAGAAGGAGAATCACAGCCCAATTTATCCCACAGCCACCATAAA
ATGCTCCTTGCATGAGCTTTTTCTCAGTCTCTGAGACGCCCTCACCTAGAATCTCCACAGTCATCTTAAA
GGATGAGAGTGGTGGGATCCTCAGTCCCTGAGATGAGATTTCTGGTATAAAAAGGATCCAATTACAGAGG
ACATATAGGAAGTCTGGAAAGTCTTGCGATATCCAGAAGGCTCACTTTTCCTGCTACCTTGTAGCAGGAC
AAATAAGTTCAGTCAACATCCCACTGTGGGCTTGGTACACAGTGAGCAAGACACTTGAAATGCAGTTTGT
GAATGAGTCCCAGAGGTCAACAGCCAGGTGAACTCTCAACATCTGGAGAGTCTTGACATTATTGTAAATC
AGAGGTGTGTGTGGTCTTATCCATGCCTCTTAGTTTTAGTTGCCTCATGAGAAAGGTGTGGGTTTTCTAT
CCTATACATAAATGTTTAATGGTTTCTTGATTCTCTTTCAGAGAGCTCTAATTGTGCCATCCCATCACAA
CAAGGTGAGTTGAAACGGGTAATCTCGGGCACAGGAAGGTAAATTCTCATTGTTGGGAGAAGTGGTTAGG
CAAATTTATAAGTCAATGGAGAAAGCCAATTTTAGAAGGCAAAGTGAGATGAAAGAATAGTAATTGATCA
GGAGTGTGTGTGTGTGTGTGTGTGTGTGTGTGTGGTGACTCAGATACAGGGTTACTCATCTCCTGTTAAT
ATGTAGGTATTGTCTCTATAAGGATGCATGAGGGAACCCAAGGGTTGAGAATTTATGAATGGTCTCCAAA
CAAATCTTTGAACAAAGCAGTAACTAGAACATCTGTTGACAAAGCTAGGATTTCTTGCCCCTGTCAACTG
ATCCTGGGGACAATCTAGGTTCTTCCCATTGCTTCCTACCATTTTGAATCCTATGAAGTCTTCAGAGTTT
TAGATGAGAGTTAGCACAGACCTTGTCTATGTAGTCCTTGCAGGACTGAATGGAGCACTTCAAAATGCTG
ATGTCTAAGTGCATGGAATTCCATCCAGGCTGGGCCCAGGAAACCAGACTAGCCCATCTCTTACTAAGGC
TTTGATTAGGAGTGGATCTGAAAAGATGGCACCAGAGCAAACAGGAATTCAAATGCCAAAGTGACCCTGA
GTCCTAGTGTTCAGTCTCCTTAGGTTGTATTCCACAGAACTATGAGTGTACAATTCTGTAGTCTTGTCCC
TGCAGCTAAACTTAACCTCCCAGTTTTTAGCATATCTAGAAGGAGCCATTATTATTCCACAGATTGTTTT
CAATTGGAGAAAGTATATTGAATTAATTAAAACAGAAAACAGAATCACAGGTGTTCCAGCCCCAACTGAG
GTCACATTTGTGCTATAGAAAATTACAACCAATGTGTTGTTGGTATTTCCTTTTTTATGTTTATCCTGGT
ATAAAATCATATTCTTATTATCAGAGGGAATCTGGCCTGATTAACCCCAATACATGTTCATATACAAAAT
GCCAGAGTTCCATTAACTTTTCTGTACCCATGTCTTTGATTTAATAATTTGGAAAGTGAGTTACTGGCTT
TTAACTTCAATAACCTTATGCATACAGGTAAGGGCTGTATGTTAGCCTTCTTCCTAATGATTATATGTTA
ATATTTGCTATTTTATGTGTATACATGTATATGTGGGCACATGTGCACACTTGTGATGGGGCATGTAGAG
GTCAGAAGTCAACGTCAAGTGGAATTTCTCAGAAGTCATCCACTTTGTTTTATGAGATGGGATCTTTGTT
GGTCTGGAGTAAGTTAAGCTGGCTTACTAGTAAGGCCCAGGAATTTGTCTGTACCCATCTTCCTAGTAAT
GAGATTATAAGCACATGCCACCATGCCTGGCTTTTATCTTACATCAGTTCTGGGGACCGAACTGCTTACA
TGGCAAGAACTTTACCAACCAAGTCATTTCCTGAGCCCATTATTTTATTTTTAAATCAATACATCAAAGC
ATTTTTAATATGGACATAATCACGGGGCACCATGTGATCTTAGTACTTTGCTTTTAAGATTCTTCCTGCA
AGACTGTCCTGTTCTTCACCGGAGTTAGATGTTGTGGAATTCCTTTCACGACACAATGCTATTTAGGACA
TCCTCGCTGTCCTAGGGAGACAGTGGCTTCTGTTGGAAGCAGATACTTTGGGAGGCCCCAGCGATATTAA
TTGTTCTTTTCAGGTATCTTTCCTAAAGGGTATTATGCCGTGGCTGTGGTGAAGGCATCGGACACTAGCA
TCACCTGGAACAACCTGAAAGGCAAGAAGTCCTGCCACACTGGGGTAGACAGAACCGCTGGTTGGAACAT
CCCTATGGGCATGCTGTACAACAGGATCAACCACTGCAAATTCGGTGAGCAGAGCTGGGAAGGCAGCTTG
GGAGGAGCCGTGCAGAAAAACAGGGTCCTGAGGGAGGCGGTGGGCTCAGGGTTCATAAAAGTGAGTTTCA
CAAAAATGAACCTCAAGGAAATAGCAAAAAAAGCTAGGAGGGGTGCGGAGCTGAGCCATGGAGTCAGGAT
GATTGTCTCAGTGGCAAATGATGGCGTCAGTAGATATACATTTAGTTGCCTCTCCCATGATGGAGATCAG
AGGTGAGCAGTCCACGGCTGATAAGGTCTTGAGAGCTTCTCCTTCTTTCCGGTCCACGATTTCCATCATA
GTACTTCCATGTTCAAAGCAACCTTGCCTGGGCAGTCACAATTCAGGCAAGGAGAACAAGTGGAAATTTA
CACCCACTATGGTCAGTGCTTGAAAAGAGCCTTCTGGACTTGAAAGCAATTATTTGCATATAACTTGCCA
TTGTGCAGAACTTAGGTGTCCTGTCAGACACCTACCTACATGTACCTACTATAAACCAAATTAGATTTTT
GTTACGGAGGAACAAAGGGAAACAGACATTGGGTGGATATCAAGCAATCTCTGCCATCAGGGTTCTGTGA
TATGACTATGTGGGCTGCCGTTTCCTGGGTCCCTTAAGGTGTTGGTCATCTCACCGAGTCCCTGCTCAGC
CCTGAGGATTCTTCAGTTGACTATGTCCTCTACCTGAGGCTGTTTCTATCTCCTTATGTTTCTGAGTAGA
AGATCCCAACAGAAAATTTTGGCCATGGCTTTGCTTCATATTGGCTTCCTCAGATCTTCTTTCCTGATGC
CTCTAAATTTGGGCAACTCCTTAAAAATGATAGAACTGTAAACATGACTATGAACTCTGCGACCTTAGAA
TATGCAGGGAGGGGAGATGGGGACACATTTCTGCACAGAGATGCTCTGCTGTCTCAGAAACAAAGGACAC
CTGGGGTGACAGGCAGAAAGCACAGGCTCCTGAACTTGCTTCCCTGAGCCTCCTTATATTTTCACATCCA
TAGAAGCAGCTGATGGGCAGAGGCTGTCTCCTATCCCTCTCTTGATTTCTGAGGTGTAGTGTAGCTCTTC
TAATGAATGGCAGGGAAGGGGAAACTCACAAAGAAAGAAAGAATGGTACAACCTTAGAAATTCCATTTCT
TCATTCTACAGGCTAGAGATGGTACCATCACAGTCCTCCAGGGGTCTCCTTTACCCTGAAGTGGAATGCT
CACAAAATCCTTTCCCAACCTTGAGAAGTTTCCTCCAAAGACCTATAGTCTGGGTGGTTCTGGGTTGGTG
GGTGGCAGGTTGCAGAGTGAATTATTTCATAGCCTGTCAGGGGTTAGGTAAGCCACTAGAATTAACACAC
ACACACACACACACACACACACACACACACACAAAGCTGACATTTTCTTGTTCTTCTGTCCAGATGAATT
TTTCAGTCAAGGCTGCGCTCCCGGGTATGAGAAGAATTCCACCCTCTGTGACCTGTGTATTGGCCCACTC
AAATGTGCTCCGAACAACAAAGAGGAATATAATGGTTACACAGGGGCTTTCAGGTGAGTTTTCCAACACA
CACACACAGAGTTGGATATCTAAAATCCACATAAAAACCAAGCACGAATATATGTCTTTAACCTCAGCAC
TTGCGAGTGTGGGATGGAGGAGAAACAAGTGGGTCTCTGGAGCTCACTAGCCACCAGCTTAGCCAAACTG
AGGCGCTCCAGGCTCAGGGAGAGACCATGTATGTCTCAAACAGTAAGGTGGGGAATGATAAAGGCAGATG
TCCAATGTTGACTTCTGACCTCTGCACACATGTACACATGTGCACATGCACACATACTCACATGAACGTG
CATATACGCCTTCCTCTCGCACCCACAAAGAATAAAGAAAAAAAAAAGCATGGTGTGTGGAGCTGAGCTT
GTGGGACTGACTGCACAGCAACTTGGGGCCTTTTGCTTTCCTTATTCTGCATGTTGTTGAGTGATAGAAC
GAGTGGCCCTAACACTTGATGACATCAGGTGACCAACCTGAATCCCCAAGGTGCCTCTGAATGACAGGTG
CCAGTCTGACCTCCTGTCTTCTCTCTTCTCCAAATCTGGACCTGTTCAGGTGTCTCGTTGAGAAAGGAGA
TGTAGCCTTTGTGAAACACCAGACTGTCCTGGATAACACCGAAGGTATGTGTGCGCTCTCTTCTGTTGGT
TCCCTCCAGAATCTGGGTCACTGGGATTTGCTGAGACTCAGGGATGGCTCCATAGGAGCTGTGCAGAAAC
CGTCAGGCTCGAGAGTTCTAATTCTCAACTTGTATGTTCAATGGAGAGAGAAGCAATTGCTCTGCAGTGA
TCCCTATGGCAAGGGTCCCTGCAAGGCTGGGGCAGGGTGAATATGCGTGGGAAGCTGCTTGCACTGCCGT
GTGCAAAGGAGGATGGGATACAGCACAGGGAGCCAGTGTATAGCATCTCCCAGTGCCATGTCTGGAAATG
TTGGACATGAAACTAATGTTAACAGTAACAGATGGCAAACCATAGATTAAAGTAAGAATCCATGATGCAA
CCCAGACTAGATAAACGATGAAGAGATAGCTCTTGCGGGCCATGGAATGCTAGCTAAGGAATGAGGTGGG
ATGGCAAGGTTGGGAAGATGGGCTTGAGTATCAGGAAGGATGAGCACGCCAGGCCTTGTATGTCACCAAG
GGCTCCTGAGGAACTGCCGTTCCGGCCATTAACCACTGCCCTGTCAGCTCTCAGGTCTGTTCTCTTCACT
GCCTTGGGCAACTGATGTAGGATGGACTGTCTCTCGGCACCAGCTCTTCTGAGACGCCTTCCCAGGCGGC
ACACCCACTCTGCTGAGAATCTTCTGTCTATGTGGCTACAAAGTATCTGTGTGCACTTTGGCTGTGGGCA
ATCACTGGGCTCTTTCTCTTCCTCTTTATCCTCCCAGCACCTGAGACTGCACTCAGAAGACACTAAGAAT
TTATTTAACGAACTGATTTGATTGTGTTCTCTCTCTCTCTCTCTCTCTCTCTCTCTCTCTTACACACACA
CACACACATTTTCTCTCTCCCCTTCCCACCTCTCTCTCCCTTCCTCTTTCCCCTTTCCCTCCCTCTCTCT
TTCTCTCCCCTCTGCTTATCTCTTTCTCTCTCCCTACCTCTCTCTCCCTCCTCCTCTCCTTCTCCTTTCC
TCTCTCTATCCCTTCCTCCCTCTCTCCCCTCTTCCTACCTTGTTCTCTCTCTTCCTCCCTCTCCCCTCTC
TCTCAGTCCCCCCTTTCTCTCTCCCTTCTCTCCCTCCCTTGTTCCCCACATGATTTTTAAACTTTCTTTA
AAGGCTTTCTTTCATGCCACCCAAGTGTCATCCTGGTCTTGGTTCTTTGTCACCTTTGATTTCTTCTGTG
CTTTTCTGAGTAAGTCTTTGATTTCTAGGCCTTGACTCTCCACTCTTCATAAAGGCTCCAACACCTGTAA
CCTCCTGGTTACAGTTGCTGTAGGAGACCTGACCTTCCATCTCCTCAGCTCCTCAGACTGACAAGCTGCT
ATTTTTATGCACAGGATGCTGGCACGACAAGCTCAGTTGGCACAAATGCAAGCCCTTCCTAGTTCTCTTT
CCATAGCTTCTGTCTCTAGAGAACCTTGTTATAATCCTCTGCCAGTCCCTCCCATTGTCCCTCCATCAGC
CCACCTTAGTACTTGGAAATTAACCCAAGGCCTGGATCATGCTTAATCAAGTCTTTACCACGTAGGGTGG
TGGAAGCAATTTGATTATATTAAAATTAAGCTGAATTAACTGTTCTACATCTACATAAGCACATTCATAT
TCAATAGACACTCATCATTACCGTAGTGGCAAAAAAAAAAAAAGTATAATTACTCCAAATATCTATTAAT
GGAGGAATGGTTTAAAAACACTGTGTTATTGTCACACTATAGAATAAGATCCAATGATTTTAAAGAATTA
CCTTGATTTGCATAATGGTGCAGAAGAAACTGTGACGCATATAAGTAGCTGAGAGCTACAGAAGTTCATA
TGTGTTAAGATCTACTGCTAAGAAAAAAAATGTAGGCTTGTAACATCACACAGACTTGTATATAGGTAAA
TGAGGACAAGGCGCATGCGTGGAAGCACAACTTGTGATTTCCAGGCTTGGTGCCAGAGAAAACATTGGGA
GTAAAGGAACGCAGAAGTGAGGGTGTTTAGTTTGTGAGGGAATAATTTTAAAATATGATAACGCTGGTCT
CACACAGTATTTGTTCAATTACGGGAAAAGCTGAGCGGACAGTGAATGAATCGAGAGAGGTCACGATCCC
GCTTTATGAGCGAGAAATGAAAACTGGCAATAAAAAATGCCCGGAGGAACCGCGGAAAACCTCCGAAAGG
GCGGAGGTCTTTAAATCGACTGTTCGGTTGGTTAACTGTTGCAAAGAGAATGCAGAAAAGATTAAGTAAG
CAGCTGAGATAGGACATTAGCTCCTGGGACTAGCATGAAGTCAAACTGAAAATATCGATGAGTATCGATA
TGAGAGCGTCTTCCGGGCGGGTCTTCCGTTTGGAGAGCTGAGAGGGCAGCACAGAGGCGGGGAAACTGCC
TTAGAGCACCACCTTCTCCCCGCTCCTTGATGGAGCTAGAAGTACAGAAGGTCTCGGGATTTTGTCTGAG
ACAGGCTTCACTCTGTATCTCCCGTTAACCTAGAATTCACTGTGTAGACCAGCTGGCCCCACATTCACAG
AGATCCATTTGTTTCTGCCTTCCAAGTGCTAAGATTAAAGGCATGCATTAGCACACCTGGCTTGCTTGCT
TGCTTGCTTGCTTTATTTATTTATTTATTTATTTAAATAAATGCATTGTTGTTGTTTAGCATTGGAATTT
AATAGAAGTTTTTTTTCTTTTTCTTTAAAAAAAAACCACCCATAGAACATGGCTTTTTTTTTTTCTTTCT
TTGTTTTCTTTTCTTTTCTTTTTGTTTTGTTTTGTTATTGTTGCTGTTGTTGTTGTTTTCAAGACAGGGT
TTCTCTGTGTAGCCCTGGCTGTCCTGGAACTCACTCTGTAGACCAGGCTGGCCCCTCGAACTCAGAAATC
CACCTGCCTCTGCCTCCCAAGTCCTGGTATTAGAGGCGAGCACCACCACTGCCTGGCTTGTGGCTCTTTT
TTCATTTGGCTGAGAGGGCAGGTCAGAGGAGAGTGGTCACTAAAAAGCTCTTGTGAATCGAGTGCTACCT
GTAATGAACACGACCATCCTCTCCTCCTAACACCACCATAACCTTAAACATGGGCTTGTGCTGGACAGAA
GGCACACGCCTTCCATACACCTGTGGGTCAGTGACATCCCAGTGACTACAACGGTTCCACCTGGCGGATC
CACCCCTTTTCTCTACCCAGGAAAGAACCCTGCCGAATGGGCTAAGAATCTGAAGCAGGAAGACTTCGAG
TTGCTCTGCCCTGATGGCACCAGGAAGCCTGTGAAAGATTTTGCCAGCTGCCACCTGGCCCAAGCTCCAA
ACCATGTTGTGGTCTCACGAAAAGAGAAGGCAGCCCGGGTTAAGGCTGTACTGACTAGCCAGGAGGTATG
AACTACCCAGGGTCTGCTACCGTTTATTCCTAGCAGGGGCAGGCTATTCGGAGTTAAACGGAGCTCCGGA
GTGAGCATTTCCTTTCTAAAAACTAGAATTTGGACTCTACAGACCGTGGATCAGAATATCTGAGTTCAAA
TCTCAGCTCGGCAGCTTTCGGTCCACGCCATCTTAGGCACATGGCTTAAAATGTCCACGTTCGGTTTCTT
CACGTATTAAATGGAAATAATACCACATACACACATGTTTCTAAGTTAACAAAAAAATAAATAAATAAAG
TGGTGCATGCACACGTCTTAGGGAATGTGTGTTTACAGGGAGGGCACACAGTGTGCTAGCTGGGACTGGA
TTCCTCAGACCTGGCTCTGCTCCTGTGGAACTCCTAGTCAGGACAGACAAATTCCCACTTGACCCATAAC
CCCTCCGAGCCTAGAATCTGCCTTCCAGCTCATCTGGATCTTGACTCGGGACTTTATTTTCCTCGAGAAT
GTTTTTCTGAAAACAATTTCTAATGTTCCTTCGTATGAGTCTTAAGGACCGGAATTGACTACTCCTTCTC
TTGCTTGTATCAGTTGCACCATATTCTTAACAGTCTGGAAAATACAAGACTCTTACTCTCAGAAAAATCC
TGTCAGGGCCAGCCTTGGGGGGCAGCGAGTACAGGGGCCACTGGAACACTTTATCCCTAGGGCCACTCTA
CACACACACACACACACACACACACACAAATTACCCTTCCTTCTGGTATAGCTGCCTGCCTTTTAATTTG
TCATTAGTTTTAAAAGAAATAGGCTAGCATACTTTGAAAAGTTCCTTCCAAAGCCTATGATAATCTTCAG
TGGTGTTTCTAGTTTGACATTAGATTTGAGTCAAGCAGTCTTACAATCACTAACAGTTTCATAGCAGCAT
TACCAAATATTTAAAGAAACTCAATGTGAAATGTTTAACCTTTCTACAGCGTTTTATACCTCTGTTTCTT
GTGTCGATTTTTAAAAAAATGCCATGTCAATTATAAATATCTTCTGTAGCTTTCTGTTGTTCCCAGGGCT
GGCTTGGACCTTTCAGTTTCCTGCAGGTGTTTCCAGTCCGAGACATAGGTTCACGTCTATTTTCTGCTCA
TTTGACAGACTTTATTTGGGGGAAGTGACTGCACCGGCAATTTCTGTTTGTTCAAGTCCACCACCAAGGA
CCTTCTGTTCAGGGATGACACCAAATGTTTCGTTAAACTTCCAGAGGGTACCACACCTGAAAAATACTTA
GGAGCGGAGTACATGCAATCTGTCGGTAACATGAGGAAGTGCTCAACCTCACGTAAGTAGAATGAACCCT
GTGGAGAGGTCGCAGAGAAACCCAGCGATGGGTGCTGGAATGGTAGTTACAAGAACTCAAACCCTTAGGA
AACAGCCACGTGAGCAAGGCAAAACCTAGCATATACAACCACGTGGGTTATATGCACATATTGTTAAGGG
GTCGCAGACCCAGGATACTAAGGCAGTGGACGAATTATATGCACACAATTCCTAAGAAGATAAAAAGGGG
CACTGAACTTATTAGAGGTCCCTGTCCAATATGGCAATGGCTAGCAAGGTGTGACAGAGCAAGTTCAGGT
GACAGCTGGATTAAATCTAGATATCATATCCTCACTCCACCCAGCTACAATTTTAATTGAAAGTAGCCAT
AGCAAGAAGAATTATCACAGAGCATCCTTTTAGACAGCTCTGTTCATAGAACATCCTTTTAGACAGCTCT
GTTCTAGGGAACTTCAATAATGATAAGTTCTGGCTAGCTAGGAAGTTGCTAAGCAGCATCTGCAATGCCA
TATTCCAGGAATTGAAATAGGATAAAATATATTTTACCATCTGTTTGGGGATTAGCGGGGACACCTGAAA
ACATTTGTAAAGTGCGAGGGGGTGCATGGGATATACAGTGGTGAGTCTCCCACTGGAGTTTGGAGGTGAG
TCTTGGCCTTCATTTAAGTTATTGTGCTGGAGATACAGCCTCGGAGACTGTGCTGCTCTGGGGTGATGGC
CCTTTCAAGGTTGCCCCATGTGGATTGGTAAGATGGCTCAGTGGGGAAAAGAGCCCGTGGCACAAGCTAG
ATGACCTGAGTTTAATCCCTAGATCTGACGGTCGGAGACAATATACTATACTATACTACACTGTGCTGCA
CTGTGCCAAGCTATGCTATATATGTGTTTTACAAGTTGCCACATGGAATTCTAGTGTGAGCAATGTGATT
GTTGAGGTTTAGTGTGACAGAATCCAAGTTAATATTTCCCTTACCTGTATGCTCCAGAGAACACCAGTCT
AGCTTTGTTTTTCTCCACTCCATCCTTAGACCCAGCTCAGACGTTGCTGGGGACTGCCAGCTACCATCTG
CCTCGCATTCTCAGGGAAGAAGGGCCTTCCTAGGCGGCTTCTCCTTATGTGAATTCAAGTTCAGAGCCTC
ACTGGTTAAAAGCAAAGTTGGGGATGCCTGTAGGGCAGGAAAGTCACATGACAGCTTCTCTATCCTTGTG
TGGAGCCCTCCCCGTGGGAGGCTCAGGAGCTGTGTAGGGAAGCAGGGATGGGAGGGATGGGGCACAGCAT
GCATGAGTCTAGCAGAACAATGAGCCTTGGCACAATTTATGTGATACTGAATTGCCCATTCAGGTTCCTT
TCATTCTGCTAAACAGCTCATAAAAGTAAGAGTATTCTCAATGTCCTATATCTTTCTTCTCTCCCCAGGA
CTCCTGGAAGCCTGCACTTTCCACAAACATTAAAATCCAAGAGGTGGGTTGCCACTGTGGTGGAGACAGA
TGCTCCCTCCCGTGGCCCATGGGCTTCTCTTGGTCTTCATGCCCTGAGGGGTTGGGGCTAACTGGTGTAG
TCTTCGCTGCTGTGCCTTACCACATACACAGAGCACAAAATAAAAACGACTGCTGACTTTA

1300017J0RIK is a predicted gene located on chromosome 9 of Mus musculus strain C57BL/6J. An exemplary DNA sequence of the 1300017J0RIK gene in Mus musculus strain C57BL/6J chromosome 9, GRCm38.p1 C57BL/6J is provided below.

NC_000075.6 (GI: 372099101: c103288297-103250530)
(SEQ ID NO: 10)
AGTCAACACTGCCTTCTGGGTAAAGTGCACCTAGGCTGGGGGAGAGCAAGCAGAACGCAGTTCCACAGTT
GAAAATGAGGCTGCTCATCTGCGCCCTTCTATGCTTGGGGACCCTAGGTGAGTGTGCTGAGAGCCAGGCA
GGGGGCCGGCCCTCTGGGGAGGGGACCAGGTGTCATTTGCTGACCATACACTTCAGGGATCAAATGTGGT
TGGATAGACTATGAGCCTAGAGAAGAGTAATCACTTTGGACTGGAATTCACCTGCCACGCAGTCCCCCGG
CTGTCCTTTGCCCTCACTGAGGCGGTCATAGTGGATTGTCGCTGACTCTTTTGCTTGCTTTTGGGACCCT
TTTCCTGCCACATCCTCCTTAATACCAGGGAGGTGCCTAGTCTTTTTGTAACTTGTTATGTCGCGTTTGG
TTGATATCCCTGGGAGGCCTGCCCTTTTCTGAAGGAAAATGGAGACGTAGTTGGTGACAGGGGATGTGAA
GAGGGACTCAGAGGAAGGAGGGGAAATTGCAGCAATATATGAGAGATAGGAGAGGAAAATAAAAAAAACA
AAACAAAACAAATAAACAAACAAAAAAACCCCACAACTTGTTTCACTGCTTTGGTAGGTAAAGGGGCTGA
TGTTGTGATTGACGACATATGACCTCCAGGACCAAATGGTAGAAGAGGAAATCCACTCTTGAAATCTGTC
CTCTGTAGTAATAATAAGTATAGCACATAAAATAAAAATTTAAAAAAACCAAAAAAAAAAAAAAAAAAGA
AAAGAAATCTGTCCTCTGACCCCCATGTGGCTTCCACACAATGTTAACTTTTTTTTAAAAAAAGTTATGT
TTACCCAACAAACACACAGTTGCTGGAGAAATGAGCTAACACAGCCAGAGGCAGGCAGAGTTCTACGCTC
ACAAATACAAGAAGCCCTTTCGTGGGTAGGGAGAACAGGCTCCAGGGAAGAACTCATGCACTGTGAAGAG
CTCTTCCTGTCTCAACCACGGTTGGATCCTCCTCCCTTTAGCTTTGTCTGATCTGAGACCGTGGTAAATA
ATACCTAGGGTGGCCAGAGCCCGGCAATGGTGCACAGTATATGTCTGCCTCTTCTGCCCAGCAGTGCCAT
CTGTAGCCACCCAGAGGGTCATTCGCTCTCAGAATGCAGCTGCCTGATCTCTTTCAGAATTCCCCTGAAC
TGAGCCAGTGTTCTGCTAAATAGCGTTCCGCCACCATTGCCAGCAATGTCAGACACAGACCAGAAGTTCA
TTTTCCCAACAGGCTTCATGTTTACATGTCAGAGACTAAAGGCCCCTCCGGGCTTCGTTTCTTCACGACC
TTTCTCCGGGCTACCAGACCAGGCCGGAGTCTGGAACCTTCATTCCTCAAAACTCAGTGTTCCAGTCCAA
TAAGAATACTTGTGGTGTCAAATGACAAAAAACAAAACAAAACCCCAAACCACCACTACCAACAACAACA
AAAAACCCCAGCCAAAGCAGCTTAAATAGGAAACACGTAACTGAGGGTTACATATCCTAAAAACTTGCAA
AAGTCAAGAGCCAAGCCAACGGTTTCTTCTGGGGTCCAGGAACTTTCTCCATTCAGTGAGTGACTGGAGG
CCACCAGCAGCCTTTCTCTTCCTCCTGAAAAGGTCAGAGCGTGCTCAGACCTCTTCCTGTGTCTGTCAGG
AGGCTAGTGGGGGCATGCTATTGATGACCTTGTTGATGTTATCAGACTCCACCCACCCTTAGCTTGTCTG
TACACGTCCTCTTTTTAAAATATTTCTATTTGCCAAGCCAGGACAGTGGTGACAGAGCTGTGATGCTCAG
GCAGGGAATGAATTCACCAAAGACAGAAGGAAGCAGTAGTAGAGAATTAGCTACACTGTGGCACAACCAG
TAGGCAGTAGAAACAGTTTAAACAGAAACTTTGTAAGAACTGGGGAGGGGTGCATGTGTGCTTGCTTGAG
TGTGTGTGTGTGTGTGTGTGTGTGTGTGTGTGTGTGTGTGTGTGTGTGTGTATGTGTCTGTGTGTGTGTA
TGTGTGTGTTTCTATGTAGGCAGTCCTCCATGTGTACTTGCTTGAGTGTGTGTGTGTGTGTGTCTGTGTG
TCTGTGTATTGTGTGTGTTTCTATGTAGGCAGTCCTCCATGTGTACTTGCTTGAGTGTGTGTGTGTCTGT
GTGTGTCTGTGTGTCTGTGTATTGTGTGTGTTTCTATGTAGGCAGTCCTCCATGTGTACTTGCTTGAGTG
TGTGTGTGTGTCTGTGTGTGTGTCTGTGTGTGTCTGTGTGTCTGTGTATGTGTGTGTTTCTATGTAGGCA
GTCCTCCAGCAATGGCGGCAGTTGAGGCTTATGGTTATCAACAGGTTGACCTTGAGGTTAGGATGATCTT
GCTGTGACTTAGAAATGGCTCAGTAAAACCTACCAGTCCTAACGTGACATCCATCTATCATTTTTAGCTA
GGGCGTGTTATGATGAAGTCTTTGTCATAGAGCAGTAGTCCCGTTCACAGGCAGTAGTGACTTGCTCTGA
GCCCAGCCTGCTTTGAGGCCAAAGCCGCTTTGTTCTGAGACTTGAAGTGGCTCAGCAGTGTTCCCCCAGA
ATATTGACACATTGTTTGCATACGATAAACTACACTCATTTAAAGGATATATACAGTTCGGCTAACGTGA
CCCCCCTCCATAATCGAGATAAAGAACATTTTTAACTTTTTCAAATTGCCCCTCCTCCTTTCTCGGCCAC
CCCCACCTCACCCCTGCAAACACCCTGCCTCCTAGGCTCACACATCTGGAGTGTAAGCACACATCCCTGT
AGGTTTGGCTTCTCTGTGTTTTCCATTTATGTCTTTATTGTGTGCTCTGTCTGTGTGTATGTGCTCTGCA
GGAGAAGACTTTTGGAGTTGGTTTTCTCCTTACATCTTATGGGGTCTGGGGTCAAACTCAGGTCATCAGG
TTGGGCAACAAGCACCCTTACCCTCTGCCATCTCCACGACCTGGACTACTGACTTCTCCTTCTTTTTTTT
CCCTCTTCCTCTTCCCCTTCTTCTTTTCCTCTTCTTCTTCCATCTCCTTCTCCTCCTCTTCCTTTCTCTC
CTCCTTCTCACCACGCCACCCCCGCATTAGATTGTAATTATTAAGCAAGAAGTGGAATTAAATAAGTTAT
TAATTTGGATGCAAGTGTGCAATACTCAAACCAAAGTGAACCTGGAATTTCTGGGGGGAAAAATTGTCAG
TAACACAAGTAGCCACTCCCTCCTTCCCACCAACAATACAGAGGCCTGTTGGAGGAAAGAATTGGGCTTT
GAGTGGGAGAGCAGGTAGGGCTGTTAGTTACACTTGGATTTTAGAATGATTTCTATGGCTCTCAGAATCA
AGAATCCATAATGAGTGCAGGTAAGGGCCACAACCTTGAAGTCTTGAGCAGGGATTCCAGTAGATCTAGC
TGTGCACCGCCATGTGAGGAGTCTGGCTTGGACCCCATTGGAAGCAGGGGAGATCACAGGTAAGGGCAGT
TTCCATACATAGACAATAAACATCAATAAACATCAATAACTCATAGGTATTTGAATGACCCATTCCTTGA
AACCTTCTCAATTGCTGTAGACAGCGGGGATAAAGCCCTCAATGGTGGAGGGGCTGTAATGGTTCCAAGG
GGTCTACTTCACAGAACTGGGAGTTCTCTCTCTCTCCCTCCTTCCCTCCCTCCCTTCCCCTCTCCTTCCC
CACTGCCCTCTCCCCATTTATGTGTGCTACATTCATACATTCAATCACATGTGGGTGTCCTCGTGTGCAC
ACACACTGTACTTGTGTGAGTTTGTGCGAAGGCTGGCAATTGACACTGGGTCCATTTTAGCGGCTGAAAC
CGGGTCTCTCACTGAGCCAGAAGCTCATCAACTCTGCTAGTCTAGCTAAACAAGATCCGGGTCTCTGCCT
CCTGAGTCCAGGCAGCTCCTTATCTACTGAGCCACCTCCACAGTCCCAGCGGCTTCTGTCACTTGGCATA
GTTGTTTGGAGATTTATCCAGGTGGCATATACCACATTCACTCTCTCAATGGCCAAACCCTGTATCACTG
TGCTGAAGGCAACTTTAGTTGGTCACATGTCCCCTGAGGACTTTTGTTTTGTTTTGTTTTGGTTTTTCGA
GACAGGGTTTCTCTGTGTAGCCCTGGCTGCCCTGGGACTCACTTTGTAGAGCAGGCTGGCCTCGAACTCA
GAAACCCGCCTGCCTCTGCCTCCCGAGTGCTGGGATTAAAGGTGTGCGCCACCACACCCGGCTAGTGAGA
GGCTTTTATGAATAACTGCCATGCACAGTAGTGGGCCCATCTTTGAGAGGATACGTGGTTTTGTTTCTTT
TGTATAAATATCTAAAATTATTGGGTCCTATTAGTAACTATATGTTGAGGGTTTTTGTTTGTTTCTTTCA
CTTGCTGTTTTTGAGGTTCATTCAGGACTTAAAGCATGATGGTACCTATCTACTCTCCCTTTAATGCACA
CTTGATGGTTTTTTTTAATGTTTACTTATTTATTTACTTACTTTAATGTGTATGAATGATCTGTCTTCAC
ACACACAAACCCAGAAAAGGGAATCAGATCCCATATAGATGGTTGTGAGCCACCATGTCGTTACTGGGAA
TTGAACTCAGGACTTCTGGAAGAGCAGCTAGCATTTTTTTTTTTTAAAGATTTATTTATTTATTATATGT
AAGTACACTGTAGCTGTCTTCAGACACTCCAGAAGAGGGAGTCAGATCTTGTTACGGATGGTTGTGAGCC
ACCATGTGGTTGCTGGGATTTGAACTCTGGACCATCGGAAGAGCAGTCGGGTGCTCTAACCCACTGAGCC
ATCTCACCAGCCCAGCAGCTAGCATTTTTAACCTTTAGCCCTGAATGTTTTTCTTTTAGTTATTGAAAAA
AAAAAAAAGAAAGTTTTGCTGTGAATATCTGTGTATTTGTTTGAGTTCCTGTTGTCAATTTTTAAATATA
TATTTAGGAGTGAAAATGCTGTGTCATATTGCTAATTCTGTGGTTATCTTTTGGGGGATGTTTGTTTGTT
TGTTTGGGTTTTGGTTTTTCGAGACAGGGTTTCTCTGTATAGCCCTGGCTGTCCTGGAACTCACTCTGTA
GACCAGGATGACCTCGAACTCAGAAATTCGCCTGCCTCTGCCTCCCAAGTGCTGGGATTAAAGGCTTGCG
CCACCACGCCTGACTTGGGGGACTTTAATATATCTGGAGAGAGTCAGAAAAAAAGGGCCCTGAGAATTCC
CTCAAGGAACACAGCTAATTCAGTGTGTGGATGACACACAGTTGGCTCTATTTTTTAGTATTAAGATATT
TTTAATTACACACATAGTGTGTGTGTTTGTGGGGGGGTTGTGCATATAATTGAGGTATCCACAACAGAGG
CCAGAAGATAATGTCCAGTTCCTCAGCACTGGAGTTACAGGAGGTTGTAAGCTGTGACTCAGGGGCTGGG
AACTGAACTTGGGTTCTCCAGAAGAGTTGAATGCAGACTTAACGGTGACACCATCTCTCTGTGCCATCGG
TCCAGGCCACTTAGCTCCTTGTCTTGTGTTTGATTCCTTTCTGCCAGCAGAGGAAGGTAAGTGCTGTGGG
TCCCTGACACCTCTTGTGTCTCTCCCCTAGGGCTGTGCCTGGCTCTTCCTGAGAAGACCATACGATGGTG
CGTTGTGTCAGATCATGAGGCCACTAAGTGTTCTAGTTTCCGTGACAATATGAAGAAAGTCCTTCCTGCA
GGTGGCCCAGCTGTTACCTGTGTAAGGAAAATGTCTCACCCTGAGTGCATCCGGGACATCTCGGTAAGTC
ACTGCTGTGTGAAAGAAAGCTGAAGAAAGGCTGTCCTTTTCCTGTATTTTACAGAGCTGGGGAGAACACA
GCTGTTCAGTGCTCGTCTGTCGGAAAAGTCATCATCTTAATCAAACAACTAATACAGGGTTCAAACTTAG
CAATGTAGCCATGACACAGCAAAACCTAAAACTGCATCAAGAGAATCTATAAAGACTCAAAAGTACTGGG
CTTAGCAGTTTAGAGCCCTGGCTGTTATCCCGGAGGACCAGGGTTCAATTCCCAGAACCCATATGGCAGC
TCAAAACTGTCTGTAACTCCAGTTCCAGAGGATCTGTCACCCTCTTACAGGCAAAACACCAATGCATATG
AAATAAAAATTTTAAAAATTAAAAAAAAAAGACTCAAAATGAAACAAGAAACTTTCTCTATGCAGACCAG
GCTTTTCCAGAACTCAAAGTGTCCCCGTTCCTCTGTCCCAGGCACTGGGACTCAAGGTGTAGACCACCAT
ACCTGGTGGAGTTTTAATATTAAGTACAAAAAGCTGGGGTGTTGTTGTTGTTGCTTTTGTTCTTTTAACA
AGGCTGGTCTTGAACTATATGCCTGCCTTTGCTTCCCGAGTGCAGGGATTAAAGGTGTGTACCACCACTA
CCCAGCTAAACTTGTTTTTTTTTTCTTAACATGAAATTTGCCTTTGTTTAATATGCATACGTGTGTGTGC
ATGTGTATGAGTGTGTATGAGAGACACAGAGACAGAGACAGAGGCAGACAAGTGGGAGAGTAAGCAGAGA
TGGATTTATAGCTATAAATATAAATTATATTTGCTAGTTGTCCTTATGCAGTTGCAGAGCAGGTTAAACT
GTCTAAGACTTGCCTTTTTCATCTGATGCTGGACCCTAAAGGGGAAGGACTCAGGAAGGGAGGATGTGCG
AAGTGGAGAAAAGCCGCCATGAACTGGAGCCTCAGAGAGGGCTGGAGCTCATGTCAAGTCCATTCATGTT
GCTCCAAATCTTCTTCCGTCCTTCTGTCTTTCTTTTATGTACGTATTTATTTTTATTTTATGAGGTCCTG
AATTCAATTCCCAGCAACCACATGGTGGCTCACAATTATCTGTAATGGGGATCCAATGCCCTCTTCTGCT
GTGTCTGAAGACAGTGACAGTGTACCCACATACATGAAATAAATAAATAAATAAATCTTTTAAAAAAATA
ATGGATATTGACAACCTAGGAAAACAAAAAAGAATGAGAGGAAGGAAGTCTGAGCTATGGAAGAGTCTGT
GGCATCAGAGTCCCGGGAAAGACACTAGCAGAGTCTTCGCCAGGGGTGATGAGTCTCATTCATGTCTGGT
CCCTTGTCTTTCAGGCCAATAAAGTAGATGCTGTGACTGTTGACGGAGCTTTGGTGGCTGAAGCTGACCT
GCCTCACCACAGCCTGAAACCTATCATGGCAGAATACTATGGATCAAAAGATGGTGTGTTCTCCCAGTAT
CACTGGTGTGGCGTGATGGCCACCATGGTTGGGGGGGGGGGGCTTTGCCAAGAGTGGCGGTTACTTTTCT
TGTTGCCGTAACAAACTGCCTAACAAAAGAAACTTCAGGAAGGGAAGGTCTATCTTGGCTCACGGTTTTA
AAGGATACAGTCCACCACTGTGGGAAGGGCAATGCCAAGGAGCGCGAGGCAGCTGGTCACATTGCATCTG
TAGTCAGGAAGCAGGGAGAAAGCGAGGGGGACTGATGCTCGGTCCACTCTCTTCTCATTTCTATTCATCC
CAGGAATGGTGCTACCCACATTCAAGGGGTGACTCTTCCCTTCTCAGTGAAACCTCTTTGAAAACCCTTA
ACGACACTCAGAGATGTGTCTCCTACATGGTTGCAAATCTAGCCAAGACAATGGTGTAAAGGAAGCATCA
TGCCAACGTTACGGGTACCAAGTCCCCAGAGCTCTTCCGATTGGCAGTGTTGTTCACTGACCCGCAGGGT
TTGGGACATTTCTCCACAGGAAGCTGTAGCTTTTATGAATCCTGATGGACAGACATATCCTTGGTTCTAT
AGTCTGTGGGACAGTTAACATCCCCAGAGGAACGTTATCTAGAGCCTATCACACACCTCTAGGGTTGTGA
CAATAAGATACCATTTCTGAGGATCCACAAGGCTCTTCTTTTTGTTGCTTCCTGTAGGCCTTATATGGCA
CAGGTGGTATGAAAAGAAGCTCTGGGGGAGTGAATGAGGGGAGATTCACCTCGCGTCCATTATTTCTATC
AGCCTGTGAAAGCACAGCCCGGCTCAGTCTGGTAGTCCACGAACACTCTCCGTCTTTCTTCTCACAACTT
TACTATGTGCCTGAGCTGTAAGCAGAGAACCACAAAAGGAAGAGAAGTCCACATAGAGACAGCAACAGTG
CAATGACTGGGACTAAAGCTTGGGGGTCCCTGGAACCCCCACGAGTCCTGAAATGAAGCATTGGAGGCTT
TGCAGACTTACAATGACTTGCAGAAACTAAATATTATAAGCCCAATGCTTTCAGTACAGCTGGTCTGGCT
AATAGCCTATCCGCAGTGCTGCGGATGAGTGTTTGTTCCCGTGGTGATCCCACGGCTAACTACACACACC
ATGGTTTAACATCTCTGTCCAGCTTCAGAGGAGGGGCACAGCATGCATTCGAAAGACCAAACTGAAAATT
CAAAGGATGCTACTGTCATCTGCATTGACCATAAAGTAAAAGCCTCACAAGTCTAGCTTGTAAGTCAGAG
AGCATCTGTGATATTAATTTATTGCCTTGGAAGGATCAGTTCTCTCTTCCTGCCAGAAAAAGGCATTTTT
TCATTCCCCACCCCACCCCACCCCCCACACTTCTTTCTAGGGAGTGGTGCTGGGCCCAGGGTCCTCTGAT
GGTCCGCATTGAGAATGCCCTGGAAGGTTGGTTGTAATGACACACTTCATTTTTGCCTTCTCTTTCTCTC
TGCTCCACTACAGATCCAAAAACCCATTATTATGTTGTGGCCATGGCAAAGAAGGGAACAGGCTTCCAAC
TGAACCAGCTCCGGGGCAAGAAGTCCTGCCATACTGGCCTGGGCTGGTCTGCTGGGTGGTACGTTCCCCT
CAGCACCCTGCTTCCTTCTGGCTCTCGGGAAACAGGTGAGACACCTGGGGCTGGGTGCCCTTAAGTGGGG
CTCCTGCACTGGATGGTCTAAATGGGTTATGTTGGGGTTCCACAATGGGTCAGTCATTTGTCTTGCTGCT
GTGAAACATACCCGACAAAAGCACCTTCAGGAAGGAAGGGCCCGAAGTCTGAGGGAACAGTCCATCTCAG
TGGGGAACTCATGATAGCAGGAGCGCTCTCTCTCTCTCTCTCTCTCTCTCTCTCTCTCTCTCTCTCTCTC
TCTCTCTCTCTCTCATTTAACCCAGGACCCAATTCCATCAGTGGGGACACACCCATGTTCAGGGTAGCTC
TTCCCTCATTAGTTAAACTTTTCTGGAAACATCTTCATAGACACACACCCAGATATGTGTTTCCATGGTG
ATTCCAAATCCAGTTGATAATGCATGTTAACCATCGCTCACTCCCTGACGAACATGTCATGCTTTATGTT
CTCCAAGATGGAGTAAGAGTATGCACTAGCATCACATAGCTGGCAGAGTCATCTCTCAATAGAGGTGCTT
CTCTTTTGCTGGGCCGTCAGTCTCTTCTTCCCTGGGAATTGCAGCAGTTTGTCTTGGCAAGGGTAGCCTT
GAAAGTCCCCAAGGAAGGTCATCTCCCTCCAAGCCTCCCAGGAAGGGTCTCTGGGGACTCCCAGAAACTT
CACCAGCCCCTCGGGTTTCCAAGTATTCATTCGAGGGCTTGGGGCATGGGCAAGGACAGTGGGACTGGTC
GATGCTAGGACATCTCTGGATACCCCAGGCTTCTCGAGCATTTAGAATGAGCCACAGTGAGCCCAGACCT
GTTCAACCCTTGAAGATGATTACCAGATCCTACACTCTGCAAAAGTGGTACCCTTTCTGAGCTGACAATG
TAGTATTCATGGATATTCTGGGGGTTTTTGTTTGTTTGTTTGTTTTAAAGATTTGTGTATTTTTATGTCT
ATGAGTACACTGTATCTGCATGTACATGTGCATGCCAGAAGAGGTCATCAGATCCTATTACAGATGGTTG
TGAGCCTCCATGTGGTTGCTGGGAACTGAACGCAGGACCTCTGGGAGAGCAGGCAGTGAGTGCCCTTAAC
CACTGAGCCACCTCTCTAGCCCCCAGTACTCGTGTTTTAACAGAGAGGGACAGGTGACTCTGTGGGTCTG
CTGGAGTCTGGTGAAGTGGAACAGCCTTGCCTTGGTGGCAGATACCCGTGGACGTCTCCCTGGGCAGCTA
GGCTACTGGCTTGCTCCTCTCCTGACTCACCAGCAGCATCTACCAAATACCCCCATGCCCCACCACCAAA
GGGATCCCATGAGGCATCATAGTCTCTGTGCACAGAAGGGATGAAGTGGGTGAGCTTCATGCAGGGAGAC
AAGGCTACTGGGTGGTGGACGATCTGCACAGGGTCTTCAGTGTTCTGTCTGGCCCTGTAAGGGGTTGGTG
AATTAACAGGGTCACGTCTTGTCTTCACGTGGGTCAGCAGTTCCCACACCTTACAGCAGACAAGCCGGCA
AGCATTTTCATGGGCTTGAGCCCTGGGCTCACCTGAGTGTGATTCTCAAACACACAGGACCCTGGGCCAT
GCATCCATTCATTCTGACGTGGGCACTGCCGACTACCTAAGGCAGGCTCCCTGCACATTCTTTCCCTTCT
TAGAAGCAGGCCTGGGAGATGTCCACAGACTTAGGCTCTCCCATAGGGCACTCCCCCACAAACATGCGTA
ACCTGGAGCCTTGAGCCAGCTCACACCCTGGGGCCTTGTCTCTGTGTTGAGCAGCGGCAGCCACGTTCTT
CTCCAGCAGCTGTGTGCCCTGTGCGGATGGAAAGATGTTCCCCAGCCTGTGCCAACTGTGTGCGGGGAAG
GGGACAGACAAGTGTGCCTGCTCCTCCCGGGAACCATACTTTGGCTCCTGGGGAGCCTTAAAGTAAGTGA
AACCTTTGCTCTGGAACAAGGGGGTCTCTTAGTGACTTTTCTCTTTTTAAAAAAAAAAAGTATTTATGAA
ATTAATTTATTATCAGATTATTAATTTACTAAAAATTAATAAATTATTAAAAAGAATTTATATTATATGT
TATATAATACTTTACATAAATAATCAAACTATGTGTATTTGTGGTTGTCTGCACGTCTGTCTGTGTGAGG
GTGTTGAATCCCTGGAACTGGAGTTACAGACAGTTGTGGGCTGCCATGCGAATGCTCGGAATTGACCCCA
GGACCTCTGGAAGACTGCTGAGTCATCTCTCCAGTCTCTTAGTTACTTTTCTTCTTTCTGTGACTAAATG
CCTGACAAGAAGCGACTTGTGAGAGTTTATTTCAGCTCACTGTTTGGGAGGATACCATCCTTCATGATGG
GAAAGGTGTGGTGGCGGGAGCAGGAGCTAGCAGGTTACCTGGAGTGGGGGATGCTGGCGCTCAGTGGGAG
CAGGGGCAGGGGCAGGAGCTAGCTGGTTACCAGGAATGGGGGATGCTGGTGTTCACTGGGAGCAGGAGCT
AACTGGTTACCAGGAGTGGGTGGGGGATGCTGGTCTCTGCTCACTTTCTCCTTTTTATTCATTGCCAAGC
TCTAGCCCATAGAGTAACACCACCCACATTCAAGGTGTGTCTTCCCACCTTAATTAACCTAGAAACACAC
TCGAAGATACGACCTGTGCTTGTCTCCTATGTTATCTGATCCAGTCTAGTTGACAACCAATATCAGCCAT
CGAAGAGAGACGAAGGCATAGGGTAGAACAGGACATACTCCTGGCATCTAGATGGCATGAGCATCAGGGT
GCAGAGTCCCTCCCGTGGCCTACCCCTGTTTCTAACCATCCTGAGGAACTATCTTCTTTGTGGGAGACCT
TATTGTCTCCCCAAAGCTGGGGCCATGACATGCCCCATGGTAACAGAAGTGCCAAGGGGTCAGACTTATC
TCTTTTCTGGAGACCGAGGTCCATAGACACAGGCCTCAGGAGTCTCCCAGCGAACCACCTTTGGCATTTT
ACCTGTGTTTTTTCATTCTCTGTAGAAGGTGACTCAAACCAAAGCAGGGTTTTGAGGTTATCTGACAGGT
TTTTGTTTGTTTGGGGTTTTTTGTTGTTGTTGTTGTTTTATTTTCATTACTGTTTTTTGAGACAGGGTTT
CTTGGTGTAACAGTCCTGGCTGTCCTGTAACTCCCTTTATGGACTAGGCTGGCCTCAAGCTCACAAAGAT
CCCCTGCCTCTGCCTCTCAAGTGTTGGAAGCAAAGTCGTGTTGTTGTTGCTGTTCAAACACTGCATTCAG
AGGCTTGGGGAAGGGTTATTTTCTGTCTGCTATTTTCTGCAAAAGAGGTCTCCCTGTCACTGAGAGCATC
TGATGCCTTTATTTTTTCTTTTCCTAATTTATTTATTTTTATTCACTTTACATCCCAATCATAGCCCCCC
TCCTGTCCTCCCAGTCCCACCCTTACAAATCCCTCACCCAATTACCCCTCCCTTTATCCTCAGAGGAGGG
GAAGGGAAGCCCCACCCTAGAATATCTAGTCCTAACAGGACTAAGCATATCCTCTCCCACTGAGGCCCAA
CACTAGGTAGGGGGAAGGGGATCCAATGGCAGGTGACAGAGACAGAGACAGCCCCTGTTTCATTCGTTAG
GGGACCCACATGAAGCCCAAGTTGCACATCTCCTACAAATGTGTGAGTGTCCTAGGTCCAGCCCCTGCAT
ACTCTTCAGTCTCTGTGAGCTTCCAGGGACCCAGGTTAGTTGACTTTGTAGGTCTTCTTGTGGTGCCATC
TATATTCCAGTCTTCCACAAGACTTCCCAAACTCCATCTGATGTTTGGCTGTGGGTCTGTCAATCAGCAT
CCCTCCGTCCACTGCCGGATGAAGCCTCTCAGAGGACAGTTTTGTTAAGCATAGCAGAGTATATCATTAA
TCGTGTCAGGGGTTGGCTCTCTCCCATGGATGGGTCTCAGGTTGGACTATTCCTTCTTTCTCTGCTCCAT
CTTTATCTCTGCACATCCTGTAGGCAGGACAAATTTGAGATTGAAGGTTTTGTGGGTAGATTGATATTAT
ATTGCTGGTCATGGTGGCTCATACCAGTAGGTTTTATCTGATGACTTGTAAGTAGCCAAAACCAAACCAG
TTGCTTGGAGCTGCTCCCTAGGAGGAATGTGAGGGTCCTGTCTGCTTCATCACCCCTCTCTGGAGCTGTC
TGCCATGATGATTTCCAGACTCCTGCCTGCTTCAGGTCTCGGCATGGCTGTATAATGTTCAAAGGCCCCA
GATAAGGCCATTGCCTCTCTGAAGGCAGATGGGCGCCACCATTTCTACCAAAGCCTTCCTAGATTGTAAG
GGGCATTGCGCAAGAGGCAGATGAACCGTGTTCCCAGGCAGAGGGCCTGACCTTGGCTGTCCTGTTTGCA
GATGTCTGCAGGATGGCACGGCGGATGTGAGCTTCGTGAAGCACCTGACAGTGTTTGGTAAGTGCTGGAC
GAAGAGCAAAGTGGGCCCCAGCCTGACCAAAGGCTCTGGCTCTTGTCTCTAAAGGGTTTTTGTTTGTTTG
TTTGCTTTTTTTTTGCTTTTAACTTCTAGAGACTATTGATATTGAGAGGCTAACTCCATTGAAGAGATTT
TATGAAAGCATTTCCACCATTTCTACATTGTTTAGGGCCCCACTTATTCTTCTTAGCACACATCCGTCTC
CTAGTGACCACACCCATTTCAGGGCTCTGCACCCATCCCTCTCTGGCTGCCTTAGCCAACCTGCTGATGC
CTCTATAAATCTAGGGAACATGAATTTTGGACGGTTATTTGCAATTTACCAACAAATGAAAGAAGTCGCT
TCTGCAGTGGCCCACATTAAACCAAGAGCCTTGTATATACCAGACAAAGGCTTTACCCTGATAACCCCCC
CATTCTAAAAATTAAAATCAAAAGTAACATGGCAAACCTTTCCACCAGCCATTTTGGACAATTTAGCTCA
AACAAGCAAAGCAACGTTTTCAGACAAGTTAAGATGACATATTCTGGTTTTGTGAGGTGTGTATGTGTGT
ATGTGACAACTGTTGGGATCATAAAAAGAGAAAGAGTTATAAGAATGTGTTCTGGCAGGGTGTGGGGAAA
GGGTGTCCCCCGGCAGACCCATGCAGAGGCATCCCTTCCCCCTGAGGGACCTGACACACACCAGTGCAGT
ATAGAATAGGGTTCATTCAGGGCATGGGGAGGGGAGTTAAGAGGGTAGCAGAGGCAGAGAAAGGCAGAGA
GGAGAGAGTAGAAACGTAGGGGCCAGCCATGACCATGTGTAGAGGAGGTGGGGGAAGGGAATGGGGAGAG
AGGGGGCAAGAGGCAAGAGGCAAGAGAGAGAGAAAAGTGTAAGAGAGAGAGAGGAGGGACCAAGCAGTCC
CTTTTATAGGGCCAGGTCTACCTGGCTGCTGCCAGGAACTGTGGGGGTAGAGTTTAGACAGAATACCAAC
AACTGTATCTGTGTGTATGTATTTGTATATGTGTCTTTCTCTCTCTGTGTATGTGTGTGTATGTATTTGT
ATATGTGTCTTTCTCTCTCTGTGTATGTGTGTGTGTGTGTATGTATTTGTATATGTGTCTTTCTCTCTCT
GTATATGTGTGTGTGTGTGTGTGTGTGTGTGTGTGTATGTATGTCTATGTATGTGTGTGTGTATGTGTGT
GTGTGTGTGTATGTGTGTGTGTGTTTATCTGTGTATGTGTTTATGTGTGTCTGTGCTGGGATTGAAGGCG
TGCGCCACCGCTGCCCAGCTAAGGCTGCAACTTTAAGATGAGGGTGTCAACAGTGCCTGCTCCTTCTGAG
GACGTGAGGAAGAATCTCCCGTCCTGTTCTCCTGGCTGGGTGGCCGTCTCTGCTTTTCCTTGGCTTGCAC
ATAAATATCGTCACTGTCTGACGTCACACTGATGTCATGCTTGCATGGCAGTCTCCCTGTGTGCACGTCT
CTGGCTCAGTTTCCTCTCCCACAAGAACACTGATTGTATTGAATTGGGAGCCCACTGTATTCCACTATGT
CATATCTTAATGGCACACACTTGTAAAGACTACTTCAAAGTGAGGTCACACTTGGGGACGCTGTGGATTA
TGACTTCAACCTATGAATGGGACAGGGTCCACAACACCCAAATCAACATAGTAGCAACCAAGGAACCTGT
GCTTCACACCTGCAGAGTTCCCTGACTGGGACCCCAGTATCCTTTCCTACCCTCGGCTCTCTCAGCACTT
TCCCCAAGCAGGCTCTGTCTCGGGGAGAACATGGCTGCGTTCATGAGGGACATGAATTGCCCCAGCATGG
CAGCATATCTGAGACCTTGACAAGTTACCTTAACTCTCTGCCTCTGTTTCTTCGTGCCTATGATGTGATC
ATTCTATGTCCAGCCTCCTTTAGCTTGGGGGATTGATGGTAAAGAGGCCTGGGCAGCAGTAAGTCTTGCA
TACAGCCGTGTGGGTCATCACTGCTAAGCGTGTGTTTGCTGCCACCATTTGTATGTTCAGATCCACAGGG
ATGTAGGCAGCTGGAGGGGGCCAGGGTCAGGCATGGTACCCTACCTGTGGGAGCCACAGTGGGACTATAA
CTCTGTCATTCCCCTGGTGAGCCCCGCTGCTCTCTCATCTGTGGAAAGTGTATTTAGAAAGGATTCTAGC
TCTGTCCCCTTCAGGCCACTCGCTCTGTGGTGGTGGGGAGAGGTCAGCACAGCTCCAGGCTCAGCCTGCT
CACCCGAACTTTCCTTCCTCAGAGGCCATGCCAACAAAGGCCGACAGGGACCAATACGAGCTGCTCTGCA
TGGACAACACCCGCAGGCCAGTGGAGGAATATGAACAGTGCTACCTGGCCCGGGTCCCTTCTCATGTTGT
GGTGGCTCGAAGTGTGGATGGCAAGGAGGACTCCATCCAAGAGCTTCTCAGAGTGGCCCAGGTACCCCAT
TCCCACATCCTTTCCTCAGCTCGAAGCTCCTGGCTTCAGTGTGTGTGGTATGTGTGGGTTTCCTCCATCT
GACCACACTAACACTAGCCGTCTTTGTGCATTCAGGATACAATGGGCGATGCATTCACCAACAATCTACC
TGCTATGTGTTTATTCCAGACAGTAGAAGCCATTAGGACCATGTGACCTACATCACCAACCCAGGGCTAA
GCACTTCCTGTCTCATTTCTGTTCTCTTTTTCATTCTATGGAGTCCCACCCTCTTCAACAAAGCACTTCC
CTTGTGACCCCCTACCCTGGCCTGTGACTCTCCTGGTGGAGGAACAGAAACAAGATCTCTGTATTGGTGG
AAATCTCTCCCAGACAGTCTCTGGACCCTATCTGTGCCAGCACTGTGTTCAGGCTCAGGGATGAAGCACC
CAGGCACAAGGAGAGCATGCATGCATTAGCACCAGGAGGAATTGCACCACCTTGAGAACATGGGCCTTGT
ACTCTGTGCTTTCACAGAGGAACTGATTGGAAGGGCATTCTGGGATAAATCCTGAGGCCTGGGGATAAGG
AGTGTGTGTGTGTGTGTGTGTGTGTGTGTGTGTCCCCATCCGTCCAAAAGAGGGGTGAAGGATTATTAGA
AAAATCAGCAGGTGGCTGATTTTATGCAGAGCCACTGCAAGAGCAAACTACAGTCATTTGAACCTTTTTG
CTATTGTTATTGTTGTTGTGCTTAGGTTTTTGTGGAGACAGGTCTCATGTAGCCTAGACTGGTCTTGACT
ATGCTAAGTAGCCAAAGGTGGACTTGCACACTCCTTATCCTCCTTCCCTACGCTCCCAAGTACTGGGAAT
ACAGACATGTGTTGCCATACCTAGCAAACACAGTCCTTTGAAAGAGCACTTGGACAGGTACCACGAAAAG
CTGAAATCAGGAACAACTGTGAGCCAGGCAACGGAGACACAGGCAACGGGCATTTGGGGTATTTTGAAAG
CCTGAAAGGAAAAGAGACTGTTTAATAACTGGTCCTTAGCAACAAGCCTGAGGTAAAGAGAAGGTCCAGG
ACACCTACCCTGCATCCCTGCTGGCAACTTCAGGGAACGTCTAGCATCACCAGGGCAACCAGTCAGGGAG
GAAAAAGTCATCACTCATGTGAACACACAGCAAGAGCCCTACTAGAGCCTACTAGAGCCGGGCAGCTGCC
TGGCACATGACCAACTCAGACCCCCCTTTCTACAGGTCGGGAGTTAGCAGTTTGCCAAGTTTATACGGCA
CAGGCTAGATGGGATTTGAGTAAAACTGTCCTTCAAAGAACTTGTTAGAAGCCCTGGAACTGTCTTTGTG
TGTGGCTATCTCCTTTTATCTGTGTGTAAGGGGTTATTTAAAAAGCTTTAGTGGATGTCTGTGATTGTAA
AAAAGTCCCAAGCCCTACAGATAGATAAATATACAGATGGATAGCTTGCCAGTTAGATGTGTAAATAAAT
AATAGATAGGGAGGTAGACACTCCTTGATTCACTGAGCTGTACTCTAGTAAACCTGTCACTGTGCTTAAA
AAAAAAATCAAATCAAACCATTGTAAGTCAGCAATCTGTGCATTCAAATGTATGATGAAGATTAATGTAT
AAATTAAGCACAATAAGGGATTATCAATAATATCAAAATACATCCATATACTACAATTAAAGTTATTTAA
AATTTAATGCCTCTGTCTGGAATTTTTCCCCTTAGTATTTTCAGAATGCTGTTAACTGTTGGCAACTGAA
ATCGCAGAAAGAATCTGTGGATACGTGTTAGTGTTATTATTAATTATTATTATCATTATTATTTTTATTA
TTATTTATCTAATTTATTTCAAGGCTCACATCTGGGCACAACTTGTAGGCATATTTGTTTTATTTATTTT
ATTTGGGTTCTTTTACCTCTACCTCCCTTTCAACCCTTATTCCTTCCAACAGCCCAACACCAGGGAGGAG
AGAAAGAAGGTTAGAGAGGACAGGAGGCGTTGGTTTCTCTTTAGAATACTTTCTGCTGACTGGGGTTGGG
TTCCTTGGAGGTGGTGGGGGAGTCTGATCTTCATAGTGAGGATATCTTCAACTTCTTCTTGTCTCTTTAC
TCTTTGCGTATGACCACTTGACTAACCCCAACAACAGCAACCAGGAGCAGCATGGAAAGGCTACAGCTGC
CACCTTCTTTCTTCTGCTCCTACTCCTCTTCCTCCTCCTCTTGCTCTTCCTCCTCCTCCTGCTCTTCTTC
CTCCTCCTGCTCTTCCTCCTCCTCCCTCCTCCTCCTCTCCCCCTCCATGTCCTCCACCACTCCTCCTCCT
CCTCCTCCTCCTCCTCCCCCCCCCATCCTCCTCTTCTGAGTCCCTCCTGGCCTCTCTTGGGGCTGCATTT
CTACCCTCTCCCGAGTCCTCAGAATTAAACTATCTGCAGCCAGCATAATTACATCCCTGCCAGAGCATGA
GGCAGGAGGTCAGCTGCTTTGAATAATCTGAAGCAGCCCCTTATCTCACACCTGGGATTAAAACAACAAC
ATGTCTACCTACATAACCTAAGTGAGATTTTAAAGAAACCAAATTCCTCACTACAGACAGGGATGGCCGC
ATGGCTTCCCACCCCCCTCCCTTACCTAGTTTGGTTTCTTTTTGTCTTTACTTTCACCTCCGTGTCAACA
ATGATCCTCTGTTTCTTTTCCTGCCTCTGGACAGGAACATTTTGGAAAAGACAAGTCATCACCCTTCCAG
CTCTTTGGCTCCCCTCACGGGGAGGACCTGCTGTTTACTGATGCTGCTCATGGGCTCCTAAGGGTCCCTC
GAAAGATAGACATCAGTCTGTACTTGGGATATGAGTTTCTTTCTGCCTTTCGGAATCTTAAAAGAGGTAT
GTCAGACTCGGGAGGGACCTGCCAAGCTTCAGCCACCGGGTGTGGCAGAGGCCTTGGATTGAGATTTTGC
TGGTGGCTGCAAGTTTCACCTAACCTTAATGGTCAGAAAAGCCCACAAGTGCTACTCAATACCCAGGGCT
AACGAGCAGATGTTCTGATAAGCCAGGAGAAAGGGCTATGCTACGCTATTTAAGGACGAGAACACTATAT
TGGTGGAAGTGGTGGTGCTGGGCTTGAACTCAGAGCCTTACAGATGTCACGAAGGGGCTTGATTACTGGG
GTATTCTGGTCCCTACATAAAGGATTTGAAAATCTTTCTATTATAGAGATTTTCTTTAAAGGTTTTGTTT
AAGTATATGAGTACACACCAGAAGAGGGCCTCAGATCCCACTGCAGATGGTTGTGAGCCACCATGTGGTT
TCTGGGAACTGAACTCAGGACCTCTGGAAGAGCAGCCAGTGCTCTTAACCACTGAGCCATCTCCAGCTCC
CTACCATAGACAATTTAAAGCCACGGTGGTTCCAGGTTCTTGTGTTAACTGAGTCTATTCTCCCAGTGGT
GGGTTTGCTCAACAGAGAGGACAATTCATCCTCAACTGACCAGGAAACACATCCATGATCACAGTCAGGA
GCAGAGCTAGAACAGGCATTGCGGAGGCTTTGAGTGCAGCGCCACCCTCGCACCCCCAACCCCCGCTTCC
ACTGGCCTCTGCTGCCCGCCCCTCTCTTCACTTCTCTGTTTTCAACCCCCAGCCCCCCTGCCCCCCAATT
TAGTCAAACAATAGTCACTGAGCATCTACAAAAGAAAGTGTTAGATGTTAGATGGGAGCTGACACAGCAG
ATGGGCCACAGCGATGAGACCATCCAAGCAGAGCACATGACTGCCATGGGCTGCTCCCTGGATGGAAGTT
CTGGAAGGGAGAGCTAAGACGCTTCCCTGGGGAGGTGATATCAGGAGGGAGGGGAAAACTCCACCTCAGC
TGCTTTATGAATGCCTAGGTCTCTACTTCTCCTAGACAATGACTGATGGCAAGGAGCTATGGCAAGGAAT
GAGTCGCACCCGCTCTTTGCCATCAGTGATGCCCTGCCTGGCATCATGTCTGCAGCCATCTGGGGCTTTC
TTTGGGTCCTTGGATCATCTTTCTGCATCCTAGGTCTGGAAGACTCCCAGAGGGTGAAGTGGTGTGCAGT
GGGCCAGCAGGAGAGGACCAAATGTGACCAGTGGAGTGCTGTGAGCGGTGGTGCTTTGGCGTGTGCCACG
GAGGAGACCCCTGAGGACTGCATCGCTGCCACCATGGTAGGAAACCCCGCTCCTTCATCTGGGCTACATT
GCATATAACTATTTTATCTCAGGGATCATACTTACAACTATAAGAATACATTAGTTGCTCTATTTAGAAT
CTTGATTAAAATATCAAGTTAATTATGAATGACTTTCTTAATGGTTATTCATTATTTTGTTAGTTATTTA
TTGAAAACGTTTCTTGCACCGCAGTTATGAGTGTGGTGCTGATGGGATTTAGGCAATGGATTTGATTCCA
TTAGTTGTAAGAAAACATTTTCCCGTGAGACAGATTAAATATAGTACCTGAATCTGACCATGGGGGTTCT
GAATTTACTGACAATGGTCATAAAGAAAGTGTGTTCTGGGCATGTCAGAGGGTCCTCTAGGAGGACACAG
GGATGTCCAGAGAGCATGGTTTGAATCGGGCATGTTTCTAGCTGACAGGCCAGAGGCAGGTCTTGTTTTC
TAAACTCCGTATTTCCACCAGTTCTGCTGTCTGGTTAAATAAGGACCCATGACAGAATGTGCATGAGATG
AAGTCTCTGAGGGTCAGAGGAATGAGAGCCACACAGTTTACGACTGATTTCTTTCTTAACTTCAGAAAGG
AGAAGCGGACGCCATGAGCCTGGATGGAGGGTTTGCCTACGTCGCAGGCCACTGCGGCCTGGTACCTGTC
CTGGCAGAGAACTACTGTGAGTGGGGCGCTTCTCTTCATTTCTCCCTGGGCCATGGGCACAGAAAGGTGA
AGAAGTAATGTCATTTTCTGGTAAGATGAGGATGACTATTTTTGCAGCTTAGGGCTCTTTGAAGTGGAAT
CTTGGACTCCCCAGTCAGAAGCAGATAAGCTGCAGATGTGGGTGTTAGGCGTCTTTCCATAGGTCTTAGA
ATCAAGCAGCCAAAGTGAGGGCAACAAGTGTCTGTCTCTCCAGTGTCTTTGCCCCTGCTGGGACAGTTAC
AAGCAAGGAGAGTTCAACGTCATACCTCCTGTTTTATCTAGAATGATACTGGAGGGCATGTGGTGGGTAA
AGCCACCACAATGCTTGTCAACTAGCCCTTGGGTTAGACTTGCTGCTAGCTCCCGGGTCTCAGCAGCCAT
GACAGGCTAGGCTCAGCCACTCCCTCAGATAACAATTAAAGAGGAGAGAGAGAGAGAGAGAGAGAGAGAG
AGAGAGAGAGATTGATTGATCTTATCTACCATAGCCACTTAAGGAAGCGGACCCATTAGAGCTGCAACCC
CTAAGTCCATCTTCAGGATTCTGATATGAAGGTTCAGGTTTAAATAAGAGGGCAAGAGACATGCATGGAT
GATCAATCCTTGTCATGATGTGGTCTCCCACATCATGGATCCATCATGCTATTTGGCTTTAGTCTCACCT
ACAAGGTGGACTGACAAGGTGGACTGACAGGGTGGTAGAACTGTATGAAACCTCTCCCTGGGAGACAAGT
TCTTCCTCAGAGACTTGCAGTGACTCTTGAGTCATTCCCTTCTCCAGCATGAGATTCCTGAAGGCATTTC
AGTGCCTTGGGGACTACTGTTCCAATAGTCTGAGAGCCAAAGTGAGGGCAACAGGTATCTGTCTCTACAG
TATCTTTGGCCCCTGCTGGAACAGTTACAGGCAGAGAGAGTTTAACATCATATCTCTTATTCTGGCTAGA
ATGATACTGGAGGGCATGAAGTGGGTAGTGCTTGCCAAACAGCCCCTGGGTTAGACTTGTTGTAGCTCAG
AGAAGATGTCAGATCCAGACAGCTGCTTCAAAAGCATCTCTGCCAAGATGTGCAGCCATCTCTCTTCTGC
TCCCCTTCTATGGGCATCTTATTGTACTTTGGATCTGTTTAAGTTTTGAGATTCTGGGGGTAGAACCTGA
TGTTATAATAGGAAAGACTAGGGGGAGAAACTAAATGGGCTGCAGAACCTTTGACTGGTGATAACCAGCG
ACGGCTTGCTAAGCCAACATTCCTCATTTTATTCCATCTCAGCTGAGGTAGAGAAGCTTACAGCTCGGGT
AGAATAACTCGTGTGAGAGTTTAACGTGGACCTTCAGTGTTTCCATTTTTCTTTTTAGTGTCTACCCACA
GCAGTGGGAGGCTGGGGTCTAAGTGTGTGAACGCACCTTTGGAAGGTGAGTGAGTCAGCTTTTTGAACTT
TGGGGTTCAAAGGCAGAACCTTATCCTTGGGAAGAAGGCAAAGGAAGCCTTAAAAAATGACCAAAATCGG
GGCTGGAGAAATGGCTTAGCAGTGAAGTATGATGATTGGTCTCGAAGATGACCCAGATTTGATGCCCAGT
ACCCACATCAGGCGGCTCACAAATACCTGTAACTCCAGTTTCCTCTTTGCTGCCCTCTGCTGGCCTCTGT
CGTTCCCTTATGAATGTGGTACACATGCACTCATGCAGGCACACACACATACACATACATTAAAAAAATA
ATAACAAATAAAACCAAACTTAATAGAGTTGAAAAGAGATGCCAGGGTTCACTGTTGAAATACACAACTG
CCCTAAAGCAGCCATAATAATTGATGTCTGCAGGGCACTTGACAATTGAAAGGACTCCTGTGAACTCTGA
GACTGCTGTCTGTTCTCCTCAGCAACCTGGTAGAATGAGCATTATTGCTCCACTGGGATGTAGTTGGGGA
AACTGAGGCTCAGGGACTGGGTGTCTTCCCAGGGGGAAAGAGCCAGGGGTGGCTGCAGTTAAGTTTGAGA
AGAGTTGGGGTGAGCAGACCCCCAAGTCCTCCTGGCACAGCACGGTGTTTCTGTCACGTGTCCACTCTTG
CACGAAGGTCAGCATAGAGGCAGAAGTCATCTGGGAGCAGGTTAGGCCTTGTGAGAGAAGGAGATTCCCA
GGGGCTGCTGAGGAGGAAGAAGAAAATGAGAATCACCCTGGGGTTAGCTGGAACTTAATAACAGGGGAAT
TGACCTGGTCCTTAGAGCAAACACCTTCAGAAGTTAAGTCTCCTGCCCACATCTCTCCATGGCTGATGTT
TATATTTCCCCTGAAGGATCCAGCCTTGAAATCAGGTGGAATGTGGCCATGGGTAAGCTCCCTGGACCAT
CAGCATCATTTGGGAATCCATTGGAAATGAGATTTCTTGACTTCTCTCTAATTCTACCCAGTCAAATACT
TCTGGAAGAGGCCAGAAGACTGCTTTGTTGGATTTAATGACAGAGCCCCAAGATGAATACCAGATACTTA
CTTCGGCTCAGCAGAGATCTGATGAGTTGAAGGTTTGGCTCAGTATAGCACACACAGCCTCAGGTGGGCT
TTGCCCCTGTTGAGTACCAGGCCACAGAGGTCCCCATGCTTTCATGTCCCTGCTCAACTACAGCAGTCCA
CCCTAATCTCCCTGTGATAGGAGCCTGAAGCTCCCCACTCTCTATATTCATGGCTAAGAAATCTTCTCTG
AATCGCGTCCTCCTACTCAAAACTGAGAGCTGGCTTCCATCAGCCGTGTATTCCAGTCTGATCATTTATG
GTCAGAAACAACATGTCTGCTTCAAGAAAAGTTGAAATATCCAGGAGCTGCAGTCACATGCAAAATGTAG
GTCAGCAAGGATGTTGAGAAGACAATTTAGACAGACACATGATGGCTGTCATCGAGAAGGTTCAGTCTGA
GTATGGGTGGAGAGGGCTCTGTATTTAGCCAAATGTATCAGCTATTTTCCTTGTTGCAATAGCAAAAAGC
ATCCTAATGGAGGAAGGGTTTGTTTTAGTTACAGTTGTAGGGGAGACAATCCCTCAGTGGTGAAAGCATG
GCAGAAGCCGGTCTTGTTGCATCAGCAGTCAGAAAGCAGAGAGAAATGAGTGTCAGGGCTCGGCTAGCTT
TCTCCTTTGATGGAGCCCAGGGAATGGCATCTGCTTTCCTACCTCAGTTAACCCAATCAAGACCAACTCT
CCCTGACATGCCCAGAGCTCTATCTCCAAGGTGCTTCTAAGTCATATCAAGTCAACAGTCAACATAAACA
ATCACACCAAATCTGTCTCTTATCTTTCTATATGTCTCTACTATCTCTTTTTTCTCTCCTGTATATCTCT
CCACCTCTGTCTCTCCCATCTGTTTCCCTCTTTCTTTTCTATCTCTGCATCTTTTATTTCTGTCTTTCTC
ACACAAAGTCAAAGATAGATGATAGCATTTAATGAAATAAGAATAAAAATCTTAGACCTGAAGACAGTAT
TAGTATTTTATAATAATAATAATAATAATAAATTTATTATTATTATCATCATCAGTAGTAGATAGTGTAG
CTACTTGTATTACAGAGTCCAGTCAGTGGAAATTGCTTTTCCACAAGCGAATCTAGAATTATCTCTGCCA
CTAGCACCACTGTTGGGAACCTACAGATTCATCTACTCTCCCCACTCAAAGCAGGATGCTCGGAACACAA
CAGATTCCCCCACCCCACATGACGAGTTTTAGCCATCACTGTACTAATGAGAAGTGTAGGTACATATAGA
GGCACCCAAGGTCATCTAAGATAGCTAGTGAGACAAGTCTACACCTGCCATGCTGAGCCCCAGTTTTTCC
CACATTTGAACACGGAGGGCAGGGTGTGTGTGTGTGTGTGTGTATGTGTGTGTGTGTGTGTGTGTGTGTG
TGTGTAATGGAGGTGGGGGTGAGTGACGGTATTTCCTAATTCTGAAGATTTATAACTTTATCTGTCTAAC
CTAACCAATTCCACTCTTCAGCTCTGACTTACTAATGAGCACGACTCAGTAGTGTATTCCTCAGATAGGA
AAACCTGAAGCACTAATGATAGTCCCATAAGTCAGGAAGAAGGCAGGAATGCTCACTCCCACCAGAGAGC
CACAGGATTACCTTAGAAGTAAAGGCAGCGAGATTAGAAAATGAGGGGGTCTTGCCAGAGAAGCAGGGGC
CCCCTTGGACCAGTCCCACACTCCCATTGTCACAGATCCTGAACCATGCTAAAGTCACACTTGCAGGCAA
ATGGCTATGTAGTTTCAGCACACCAGCCAAAGGATGGAGCTGAACAGAAGATGGAGAAACCAAGTTCACA
TTCTCCTCCACACAGAGACTCCATTAAATGTTCCCGGGGAAGGGAGTGTGCCAGATCCTGCTTTTGCCCA
CTCTCTGTCTCCAAACAGTAAAGAGCTTGTGTCTCGGTGTCTCCACTGCAAGGTTATTATGTTGTGGCTG
TGGTTAAGAAATCGGATGTTGGCATCACCTGGAAATCTCTGCAAGGCAAGAAGTCCTGCCACACGGCCGT
TGGCACTTCTGAAGGCTGGAACGTCCCCATGGGTTTAATATACAACCAAACTGGGTCTTGCAAATTTGGT
AAGTGTGTTCTGAAGGTAAGGCGGTGCCTTCCTTGGGCTTGTTCCTGAGTTGGGGGGATATTCAGTTCAG
GAGATGGCAAGGAATGTCCTGGCCACCTAGGATATTTGGGTCATGGGCTTGACAGAAAGTGAACTCCACC
CCTTCCCAGGTAACTCCTCCCCTTTCTCCTACTTTCACTACCTGCTTTTGATGGGAGTACATAGCCCTGA
AGGCAGATCTCAGGGAAAGGGCCCTGTTATGTCTTATGAACAGTCCTGGGAATAAATGGAGCCTTAGTAG
TGTGTGTAAGTGTGCACATGTGTGTTATTTTCACATATACCCTGAACTGACAGCTTTCACACTTGAAGCC
CTTTCACCAGGAAGGCCCTGGAGTGCTGCCTCCCCTTGCACATTTATCCTGCTCCCTTGACTCCCACCCT
AGCCACTTCAGCTGCACCCTCAAGGGTACCTCAGATTCCTTGCCTCTATCACTTGATACATCCCAGTCTT
CTTCTCTGGAACAGAGGAACCAGGCTGGAGAAGAGCCCAGGACAACAGAGTAGTCATTGCTCTGGAAAAG
CTCTGACCAGTGGCCTCAGTGGGCCCCCAACACTCCCGAGTGCAGCTGCCCCTCACACTTCCAATAATTG
TGCCTGTGTCCACACACCTCAAACCCACACCGACCTGCCACCTTCTCTGGCAGATGACCCAGTCAGTGGA
CACCATCAGAAGACAACTGAAACCATCAGCATCCATCAGCAGAAGCTGCTGAGTCTATGGACCTCCTGAC
TGCTGCCTCTCCTCAGCCTCCTACATGCCCAGATCCCTCTCCCTGCACTTGGGTACCACTCACGACAAAG
CCCAAGACACTCAACTATTTTTCTTTTCTTAGCCTATCCAACAATTCCTTTCAACACGTCAATACACTGA
TGTCTTCTATCTTTAAGATTCTTAACTCCTACAACATTCTCCTGCAGCCCCTGTCTAAGTTCACTTTTTC
CTTTCCTCCCAGATCTTAAAAACACTACAGGAGCCACACACCTTTCTCTCACATCTCCACTTCCTGCTGT
CTGTCTTCTTGCATCATCCATCCCGAGGGCATCACTCTGATGGAGACCCCCACAGTGCCCTCCAGCTGCT
CAGCTCCTGCTCTCCCAGCACCACCACCCCTCTATTCCTGGCTTTCGTGATGATGTCTAGATGTTGTTTT
TCTCCTTCTCCTAAAGGAGTCTATCCTTTCCAACCTGGTTAAGAAACATCGGGGTTTCATAAAGCAGTTC
TCTTCCCGTCTAAGCATCATCCCTAAGCACTCACATCCAAATGCAGCTGGATCATTACGCTGCCCATACA
AAACCAGGTACTCGGTATGCACTGGATAAATGCTGACTGCTGTTGCTTTTATTGTGTCTAAAGGGCGATA
ACTCTCAAGCACAGATTGCTCCTTCACACACACACACACACACACCGCAACACACACGCACACACCCCCC
CCCCTGCAGCACACACACATACACAGTCTCAAGAAGTTGGGCTTCACCTCAGACTCGCGATGCAGCTGAG
ACTGATATTGAAGCTCTGATCTTCCCGCCTCCCAAGTGCTGGGATTATAGGTGTGCATGACCACATGCAA
CCCAAACATGTATCTAATGAACTATTTCCATATCTACTTAAGATCTCGAAGGCATATTAAACTCATCTCG
ATTATCCAAAACGGTGTTCTCTCATCCTAACCTGGTGATGCTCCAGGACTGCCTAGCACCGTGGAGGACT
CTGGAGTCCCACTTGCCCAGCAACGTCAGTTCTTTCTCCTCCCACCCACGTCTTCTTACTTCACGCTCCC
TCTCCTAGCCCACTGCAGTGGCCTGCCACCCGAGCTGCACATCAACCCTACATCCATCTACTTCTCAGTC
AGACTTCTGCAAAGTGTGGTTCTGATTGTGTGTGCCAATCCACTGGGCCCAGCATTGTCTAAATCACTTC
AGAGACTCCCTTTACTCTAGAGATAAACAACAGTAGCTACACAGTAACATCCCCTGGGGTCTGAACCTTG
CCCCTGCCCTACTCCCTTCTCTTGACAGTCTATTCAAACGGCCCCCATTAAGCTTGTTATGCTATGTCTG
GGTCCTGCTCTCAGGTGCCAGTCCCCTGTCTGTAAGCTTCTCTTCTCCCCATTCCCTTGGGGACTGCCTA
CTCATTCCCCATCTCTTGACTCCCTGGTCTATGGAAGGTCCCTCTATCTCCAGTTTCAGGAAATCATATT
CCTTCTTCCCAGAGCAACGGGGGCCATGCAGATCCATGGGTGTGGTTGCTTGAGCTCTGAATGGTACCCA
CAGAGAGTAAGTGGAAAAGGGAAGGGCCCATGGTCAGTGTAGTTCCCTGGAACCCAGTGCTAGGCTTGGC
AGCCATGGCTTTCCTAACTTGAAGTCACAATGGCAAGCTTTCTAAAGGGAAAACCAAGACAGCAGAGACC
AGAACGCATGGGTTTAGGTCAAACAAGCCAGCCCACTCCCTTTCCACTATGTTAGAGTTACACTCTCTAC
CTACCCTTCAACACCCTGGTCCTGTTTGAGGACAGGTAGACTTCTGTGAGCTCTCAGGCCGGCTCATCTC
CAGTCAGTCCCTTTACGTGGTATGGAATCTTTTCTTTTCTTTTCTTTTCTTTTCTTTTCTTTTCTTTTCT
TTTCTTTTCTTTTCTTTTCTTTTCTTTTCTTCTCTTCTCTTCTCTTTTCTTTCCTCTTCTCTTCTCTTCT
CTTCTCTTTTCTCTTTCTTTCTTTCTTTTTTCTTTTTTTCTTTTTTTCTGAGACAGGGCTGTCCTGGAAC
TCACTTTGTAGACCAGGCTGGCCTTGAACTCAGAAATCCACCTGCCTCTGCCTCCCAAGTGCTGGGATTA
AAGGCGTGCACCACCACTGCCTGGCTTGGAATCTTTTCTTTTATTCCATGATGTTCTTGTTCTTTTTTAA
AAAATGAGTTTTGTATATGTAGAGTATGTATGTGTTCATATGGACATGGATATGTATATGTGCAGGTGCA
TGTATGCACACATCTATGGAAGCCAGAGATAGAGGCTGGTTGTCTTCTTCAGTTGATCTCCACATTATTA
TTATTGTTTGAGTAAGAGTCCCTCTGTGTAGCCCTGATGGTTCCGAGATCCAACTGTCCCTGCCTCCTAA
GTGCTGGTACTTATAGCTCCTGTCTTAGTTTTTGAGACTGGGTCTCTCACTGAACCTGGAGCTCACCATT
TTGACTAAGCTGGCAGGTCAGCGAGCTGCAGGGATCCACCTGTCTCTGCCTTTCTCAGCACTGGAGTTAC
AGATTTACACCACAGCACCCAGCTTTTACGTAGGTTCTGGGGATCCAAACTCAGGTCCTCTTGCTTGCGT
GGTATGCACTTTGCTGGCTGAGCCATCTCCCTAGCCCTTACTAATGTTGCTGAATAACAAGCTGTGTGCT
TGTGTTGCAGGCAGTCTCAGGGACAAACCTCCGGGGCTTTCCTGTGATCACGGGAAGAGCTGACTCTGGC
TTGTCCTTCCTTGCAGATGCATTCTTCAGTCGCAGCTGTGCCCCAGGGTCTGACCCTGACTCTCCTCTCT
GCGCTCTGTGTGTTGGGGGTAACAACCCAGCCCACATGTGTGCTGCCAACAACGCTGAGGGGTATCACGG
CTCCAGCGGAGCTCTCAGGTTAGTGTCTGTGCCTTTCCTTCTCTTGGCATCACCCTGTTGACCCCCTCAT
CTCCTCATGCATTCATCATATCAGTATCTGGGTGGTGATTTGGGAGGATCTGGGATTGGAGACCAGAGTT
AACTGCTGATATTCAGCCTGGTTCTATAAAGAATACTCCCTTGGCCCAGGTGCCTGGGCACTTCAGGGAC
TTGGATAGGGCAAGATCAGTAGTACAGAGTCTAGTTTTCCTCTTATCTTTTGTGTGACTGTAGATAAGCC
ATTGGTTCTGTTAGAGCTCAGCTGTCAATCACAGGAGAGACCCAGACTCTGGCTTCCCCCACTGAGCTCT
TTTACACCGACAGATCTTTTACAATAGACCTGTCAAGGTACTGCCTTTCATTTAAATGAAAGAAGATGCA
GTTACAGAGCACTGAGTAGCAGGTGCTCGTCTAGGTAGCTAGCAAGCAACAAGACTCGCACGGTCCTGTC
TGCTCTGTGTCCCAGTACAAAATAGGACCGTAAACAGACAAGTAAAGAAAATTGTTCTCAACTGAATATG
CTATAGAGAGACCAAGAGGAGGCACGAGTTAGGGAGCTTTCCATCCTTGTAACAAAACACCCGAGACAAT
CAGCTTATAAGACAAAAAGGTTCATTTTAGCTCATGATCTTAGTGGTTCTAAGTAGAACCAATGCAACTG
GGATCCGGCCGTCTGTTTCAAAGGCGTGTCTCAACGACTCAGGGATCACTCAGTAGGACCCATCTCCTTC
TGGTTTCACCTCTAATTACCACCACCTTAGAAATAAGCTCTTTTTTTAAAATTTAAGTTTAAATTTGATT
TTAATTTTTATGTGTGTACATGTGTCCCTGTGTGTGTGTCTGTTTAAATATGATCATATTCATACAGGTG
CCTGCAGATGCCAGAAGAGGGCATCAGACACCAGTCCAACCCCTGGAGTTGGACTTAGGGGTGATTGTCA
GCTGCCCAACATGGGGGTTGGGAAGCAAACTCTGCTTCTCTGGAAGAGCAGCAAGTGCTCTCAAACACTG
AACCATCTCTCCAGGTCCCAGAGCCAAGCCTTTGATAGTCAAGTGCCTTTGGAAGATGTTCAAGATCCTA
GCACAGCAGGGAGTAATGTGGCAATGGCCAGGGCAGGCAACAGCTTAAGAAAAGGTACATGGGGAAGGTC
TTTGAGAGCAGAGAGATCAGGAGAGCTGAGAACTAAATAATGAGAGATGAAATTGGCCAGAGAGAATCTG
GAGCAAGAGTAATCGAGAAGTGAATCAGCACGGGTGAGGCATGTGTGGCAAAGACCCTACTGAGTGAGGC
CAGCAATAAAATTCTGTGGACTGAAGTGTGGAGAAGGAAGGTGTCTGGGTTGTGACCTGCTGCCTCTGCC
TGTGGTGGCCATGTCTATTGAACCCTGGAGTCCTGGCTGAACAGGAAAACTGAGTTCTAAATATGATGAG
CCAGCCATGCTGGATATGTTCAGATTGGTGTGTGTGTGTCTGTGTGTCTGTGTGCCTGTGTGTGTGCCTG
TGTGTGTGTGCCTGTGTGTGTGTGTCTGTGTGTGTCTGTGTGTGTGTGCCTGTGTGTGTCTGTGTGTGTG
TGTATGTCTGTCTGTCTTTGTTTGTACTGGATGAGTAAAAGCTCTGTGGGCAGCATGGGGCACTCTAGAA
AACTCCCCTGCCCCCGTATACATCAGTTGACTTCATCCTGCCCTCCAAACATTTGCCTCTTATCAGTCAT
GTGCTAGTGACATCAATATCTATCTGCTGAATGTTGAATCTAAGCCAAACTAACTTACATTATCTTTATA
TCACAAGGTAACTGGTATTAAGCTTGACCCAACTTTAATACTTTTTCAAATCATTTTTATTTGCTTATTA
TTTTATTTACTTATTATTTTGTATGTGTAAGGTGCATATGCTCCAGGATATTTATATCTGTCTGTCTGTC
TATCTATCTGTCCATCTATCATCTATCTGTCTGACTATCTATCTATCTATCTATCTATCTATCTATCTAT
CTATCTATCTATCTATGTTGGAAAGGGGAGGACTTGTGCACAGCAGTGCACATGTGGAGGTCAGAAAACA
ACTTGAGGCAGTTAATTCTCTTTTTCTACCATCTGTAATCCAGAGACCAAACTCAGATTGTCAAGTTTGG
TGAGTGAGTGTTTGTTTGTTTGTTTGTTTTTGTTTTTGTTTTGTTTTTGTAAAGACAGACTCTCACTCTG
TAGCCCAGACTGGCCTGAAATTTGTGATTCTCTGGTCTCGGCCTCTCAAATGCTGGAGTTATAGATGTCG
CCACAGGTACACATTTTCCCATGGGATAAAGATTGTGTCGTGTTTAGAGAAGCAGATGAGTGCTGTCAGA
GCTGAGCAATTGAGTGCAATAAAATCACCTCACAGCCATATTTCCACGCGGATTGTAGTATCTCATGGTG
TGATACTACAGCACGTGGGTCTTGTAGATCTAACTAGAGTGCCATCCCATAGATATCTCTGTTAGTGGGA
GTACACAAGCAACCTGTCTGGGGAAAGTGGTGAGGATTTAAAACAAGGCCTGAACGGTGCTTTCCACAAG
GGAAACTCAATGAGCAGTTCTGCCTCTCCATCACCTTCCTTCCTGGGGCCCTGCAGGCACTACAGTTGCT
TGTGTGGGGTGCTGGGTGCCTAGTGGTGTGACTCCCCCTTGGTGGCTCCCTCTTACTCCTTGCTGATTCT
CCTTGCTGGCCTGGGCCTTGTTGTGCAGGTGTCTGGTTGAGAAGGGGGACGTGGCCTTCATGAAGCACCC
TACAGTTCTACAGAACACTGATGGTACGTAGGCGCCTGCAATGTGCTTTCTGTCCATGGGAACACTCGAG
TCTCCCTTGCAGAGTGGCTCCCCAAGAGGCAACTGGGGAAGTTGAATAGCTAAGCCCAGGAGACTCCAGT
GACTAGGATCGTAGCACTTTAAATTAATCTAGCATTGAAACGGTCTTCCAGTCTGTTCAGACTTGATGCT
AGTTATCACCCTGGCCTTGAACCTGGCTGACAAAGCCCACAGAAACCATGGGCTGTGTGAGTAGGTCTGA
CTCCCTGATTGACAGGCATTCCTTTGGTATTCCAGATTAAAGTTCTTGACAGCCATCCCGTGTTGGAAAA
TGTCTTGTCTGTTTCTGTAGCTTGACTTTTTTCACTTTCTTTAAAAAAATTGTCTACTTATTTATATATT
TATTTGTATGTGAGTGGTCATGAGGATTTATGCACACCACATGTGTGCAAGAGCCCAAAGCCAGAGGGCA
TTAGACCAACTGACCCTGAAGCTGCCTGAAGTTGGCTAGGAACTGAACTTGGGTCCTTGGAAGAACAGTG
AGGTCTTAACCCCTGAGCCAGATCTTTTGACCCCAGGTGGATAATATTAATGAGTTTTTGAGTTTTTTAT
ATAACTTTTAAAAAAAGATTTATTTACTTATTTTATGTACATGAGTGCTCTATCTGCATGTACACTTGAA
TACCAGAAGAAGGTATCAGGTCATATTATAGATGGTTGTGGGCCACCATGTGGTTGCTGGGAACTGAACT
TAGGACCTCTGAAAGATCAGATAGTGTTCTTAACTGCTGAGCCATCTCTCCAGCCTCTTCCTTTGTTTGT
TTGTTTTTTAATTGTTGTTTTTTGTTTTTTGGGTTGTTTGTTTGTTTGTTTGTTTGAGACAGAGTTTCTC
TATGTAGCCCTGGTTGTCTTGTAACTCACTTTGTAGACTAGGCTGGCTTTGAACTCTGAGATCTACCTGC
CTTTGCCTCCCAAGTGCTGGAATTAAAAGCGTACCCATCACTGTCTGACAACAGTCTTAAATTTTAAAAC
ACTCCAATGTGTTGAATTTTCCCCTTTGGTTAACACTCCCAGGACTGTTATTTTAAATTCTTATTCAGTT
CTGGAGAATTTAACCAAGGGTCTTGCTAGGCAAATGCTCTACAGTGAGCTGCAGCCCTGGCATTTGATTT
GTTTATTGATTAATTGATTTTGAATTTTATAGCTATGAGTCTTTTACCAGGATGTATGTCTGTATACCAC
ATGCATGCCTGTTGCCTGTAGAGGCCAAAAGAGAGCATCAGATCCCCTGGGGTTGGAGTTGCAGACACTT
GTGAGTCACCATGTGGGTGCTATGACACAAGCCCAGGTCCTCTGGTCCTTAACCTCTGAGCCATGTCTCT
AAACAATTATATCTATATTTTAAACTTACACGTATTTACTTAATGTCTGTTTGTTATGAGGTCAGAGGAA
AACTTACTTGAGTCAGTTCTCTCCCTCTATCTATTATGTGGGTCCCAAGGGTCCAACTCAGGTCCTCTGG
CTTGGCAGCAACTGCCTTCTTTCTTTCTTTCTTTCTTTCTTTCTTTCTTTCTTTCTTTCTTTCTTTCTTT
CTCTCTCTCTCTCTTTCTTTCTCTCTTTCTTTCTTTCTTTCTTTCTTTCTTTCTTTCTCTCTCTCTCTCT
CTCTCTCTCTCTCTCTCTCTCTCTCTCTTTCTCTCTTTCCCCACTGAGCCACCTCACTGGCCCCAACATC
ATTCTCTCCCTAGGAAAGAACCCTGAGCCTTGGGCTAAGGGTCTGAAACACGAAGACTTTGAGCTGCTCT
GCCTTGATGGCACCAGGAAGCCTGTGACTGAGGCTCAGAGCTGCCACCTGGCCAGAGTGCCAAACCGTGC
TGTGTTCTCCAGGAAAGATAAGGCTGACTTTGTTCGAAGAATACTCTTCAACCAACAGGTATGGGCACCA
GTGCCTACCCCCTGTGACTCAGTGCCTGGTTCCCAAGAGCTGCACTGATGGCCGTGTGCCTGTGCACCCA
GCTTAGCTCATGCTCTTTCTCCCTCAGCAGGGGGCCCTGGACCTACGAGTCCTCACTCTGGTTATATCTG
GTGCCCACTTGTTCTGCAGCTCTCTAGGAGTGTAAGATTTGCAGCCTTGAGGAAACACACAGGATCTCTT
GGGCCCGGTCAATTTCCAAGAGAATGAATCTCTTCTCTCTTCTTCTGGTGCATTAAGTAAAGGAGTTGTT
GGTTCAGTGGCTGAGCCAAGTGTAACAAAAGCAGAATGGTGGGTTCTCTAACTGACTTGTGGGTTGATCT
GAGTCTGTCTTCTTAACCTGGATAGGCTGGGCACCATCTGGGCTTGCTGAAAGTTAAGGAGCCAAACATC
TAGACACTTTCTGACTGTATTGAAAGCCAACAGTTGCTGATGTTATGGAGTATCAGATGCCTAACCCAAA
TGATCTGAAGGAAAACCTATAGCCTTCTCTGCCTACTGCAATAGATATCCCGTTCTTGGCGGTTTCTCAA
AGAAGAAATCCATGTTTTATCATATATCACCTTCACATCCCTGAAAAAAAAAAAAAAAAAAAGATGACCT
AGTGTCCTGGTGTTCCAGTTTTCTTCTCTTTGTTGTGCTAGGGCAATAACCAAAAGAAACTTGGGCACAG
AAAGGGTTTATCTGACTTACAAGTTTAATCTATCATTGGGGAAGTCAGGCAGGAAACTCAAGACAGGAAC
CTGAAGGCAGGAACTAAAACAGACCATGGAGGAGCACCACTTACTGTTTTGCTTGATAACTCGCATTGAG
CTGCCTTTCTTGTATAGTCTCAGCCAACCTGCTGAGGGATGGTACCACCCACGAAGGGCTGGGCCCTCCT
ATATCAACTATTAAGAAAATACCCATAGACATGCCCACGGGCCAATCTAAGAGACTGTAGCTAACTGAGA
ATCCCTCTTTCCAGGTATGTAACATTGACAATCAAAATTAACCTTCACACCTAGGAAAACCATTGAAATA
AGAGAGTCTTGACTGTGGACATGTGGCATAGAGGTGGGGGGGAGTCTCACACAAGATAAGCTGCCATGTT
GGCTACTATGGAAATCAACTAAGGAAGAGAAGTAGGATGGAGATATAGAACTGGTTGGAGTTCAGAGACA
AGAAGAGGAAGCAGCAAGGGAAGACATGGGAAGATTTTGCCTGTAGCTTTAGAAAATCACTACTGCCTTT
GCCTAGGACAGAAGCTGGCTCAGCTCCATTGGGGGGGGGCGTGTCTTGCTATGAACCCACTTCCAAAACC
CTTGAATCCGAAGATAAAAGACACACACAGACAGCTTTGTTACCTTAACTCGCCTGCTAGGCACAATTGC
TGGGTGCAAATATCTCCTGCCTGGAAATACATGCCCTTACCAATTTATTATCTCCATATTTCCTTCTGCC
CTAAGCTTAGTGGCTTGTTCCAAATAGCCCCTGCCAAACATCTTTGGCCACCTGCCTGTAACCGAAGCCA
CAGGCCCTAGCTTCCCCCAAGGCCTTACATGTTTGCTGTGTCCTTCTTCCTCCAAAGCATGGCAGAAAAA
GCCTCTTTCCTTCCTTTGCATCTCCCTTTTCCTCCTGGGACCCGGAAGTCCCACCTGTACTGCCTGCCCA
GCAATTAGCCCCCGGCTTTCTTTATTGACACATCAAGAGCCAATTGGGGAAATAGCGTTAGTGACAGAAC
CTCCCCCTACAGGAGGATGATCCATACAGCTGGGAAAGCACCTGGCACTGGCTGGCTAACTGGTGACATA
TGGCCAGCTTTCTACTTGTGTTTCTTATGAAAGAAAGATGAAATTTCAAGACCTGTAAGTCATATAAAGT
ACTCAGAAATTGCTGGTTGTTTGTGAGCCTAGAGGCTGCCTGGGGCTGAGAAAAAAGAAAAACAAACCTG
GGTATGCCCCGGAGTTAAAACATTCCTGGGAACAGCTTAACTTTAAAGATAAAGAGGAATGTGAGGACAG
AACAGGGCTATCTAAACTGAGTCAACAACTCACAGAACTCTGACACCCTGCACGTACATGTAATTTTTCT
GCTGATATTTGAATAAGCCAATAGTGTGCCACTATGCTGAATTCCATGCCCCTAGGCCCCTTACCCCATA
AAAACCCCTAACTTTCGAGCCTCGTGGCCGACATCCGTTATCTCCTGTGTGGGATGCATGTCGGTCCGGA
GCTCCGTCATTAAATGACCTCATGTAATTACATCAAGATGGTCCTTCGTGATTTTTTGGGTGCACGCCGA
ATCGGGAATTGAGTGGGGGTTTCCCCCACTAGGTTCTATCACTTACATACAGCATTGCACAATGTGACGT
TAGTGTCTCCATTTACAAATGAGCAAACAGAGGTAGACTGAGCGTGGTGAAGGATTTTCCTAAGTTCACA
TAGCAAGAAAAAGATGAAGCCAGGATTCAAACCCACAGACCATGCTTTCTGTTACTACAGCATTCCCTGA
ACTGTTGTTGTGTCCAGGGATTCCCCAAGGACTGGAGCTGAGCAGTCCAGAAACCTTTGGTTTTCTAGAA
AGAGTAGACACCTCTATGCTTCCTGGCACCCCTAGTGACTTTCTGTTTATTCCAATCCAGGAGCTCTTTG
GAAGAAATGGATTTGAATATATGATGTTCCAGATGTTTGAATCCTCAGCTAAGGACCTGCTTTTCAGTGA
TGACACAGAATGTTTATCTAACCTTCAGAACAAAACAACATATAAAACATACCTAGGACCACAGTATCTT
ACCTTGATGGATAACTTTAGACAGTGCTTGTCCTCTGGTAAGTAGAAACAACCAAGGAGAAATAGCAGGA
GATTCGAAATCAGCATGTTTATGTTAAGAGCTCGGTTGTTTCAAGTATTCAGTTGATACAAAGGCATGGA
GAAGCAGAAACTTTCTCTTTGACTTGAAGGTTTAGAGTTACCATTGAGCCAATTCAATTAGAAAATAATG
AAAGACACCATCCATGTAATGCTAAATTGTCTGAAGGAGACAAGAAGTGTAGCAGAACTTGAAACCATTT
GGGAAATGAAGCTCTTCTCATGTGTATTCCCATTTTTGTTGGGGCTTGGGAAATGATACAAGCTGTATGT
CTTAGGGTTTACTGTTGTGAACAGACACCGACCAAGGCAACTCTTATAAGGACAACATTTAATTGGGGCT
GACTTACAGGTTCAGAGGTTTAGTCCATTATCTTCAAAGTGGAACATGGCAGCATCCAGGCAGGCATGGT
ACAGGAGGAGCTGAGAGTTCTACATCTTTATCTGAAGTCCACTAGGAGAAGACTGACTTCCAGGCAGCTA
GGATGAGAGTCTCAAAGCCCACACCCACAGTGACACACCTTCTCCAACAAGGTCACACCTTGTGATAGTG
AGCCAAGTATATTAAAACCACCACACTGTGCATACCACCCGTGAAGGGCTGGTTTTTGATGGTTTTATAT
AAAATGCTCAAACATTAATAAAAGTAAATACAGCATGGCTAGCTCTGTAGGAATCTTGGGATACATATTT
TAGATGGCATAGATGTCTCAAAAAAATAGAAATTGGGAAAATAAAATTAAAACTGAGATAACCATTGGGC
ATATGCCCGACTCTGTGGCATTGTGTTGTGTGTTAGGGATAATGTAAGCAGGAAGGAAGGACAGGTACTT
AAGGATAGGAAGGAAGGACAGCACAGGGATTTAAGGACATGAAGGAAGGACAGATACTTAAGGATATACA
TGGTAGGACAGAGATGGCCAGTGAGGGTCTTAACTCATCTCAACTGTGGCTTACTTTTAGAAGACAGGAG
TGTTTCCAATACACTTTCAAGAATTTCACTTTTAAGAGCATGAGAAATCAGAGCAGACGAGTTTGGCAAG
TGCAGGCAGAGCCTGATGTTCTTCCCCTTGTGTTTTAATTTACAGAACTGCTGGATGCCTGCACATTTCA
TAAATATTAAACACCATTCAGCTGGGGGGGGCGGTAACCCAGGGGCTGGGGACCCAACTGCTTGCCTCCA
TGTGGACTTGTGCAGAATGAATAAAAGTATCTTCCCAG

An exemplary DNA sequence of the Trf gene in humans is provided below.

(SEQ ID NO: 23)

TGGCACCGAGCGAGCCGCGATGACAATGGCTGCATTGTGCTTCATGTCCC
TTCCCATCAACATTTCTGTGCTGGACTCCTTCCACTCGCGGGTCGTCTCC
AGAGCTCAGAAAATGAGGTGATCAGTGGGACGAGTAAGGAAGGGGGGTTG
GGAGAGGGGCGATTGGGCAACCCGGCTGCACAAACACGGGAGGTCAAAGA
TTGCGCCCAGCCCGCCCAGGCCGGGAATGGAATAAAGGGACGCGGGGCGC
CGGAGGCTGCACAGAAGCGAGTCCGACTGTGCTCGCTGCTCAGCGCCGCA
CCCGGAAGATGAGGCTCGCCGTGGGAGCCCTGCTGGTCTGCGCCGTCCTG
GGGCTGTGTCTGGCTGTCCCTGATAAAACTGTGAGATGGTGTGCAGTGTC
GGAGCATGAGGCCACTAAGTGCCAGAGTTTCCGCGACCATATGAAAAGCG
TCATTCCATCCGATGGTCCCAGTGTTGCTTGTGTGAAGAAAGCCTCCTAC
CTTGATTGCATCAGGGCCATTGCGGCAAACGAAGCGGATGCTGTGACACT
GGATGCAGGTTTGGTGTATGATGCTTACCTGGCTCCCAATAACCTGAAGC
CTGTGGTGGCAGAGTTCTATGGGTCAAAAGAGGATCCACAGACTTTCTAT
TATGCTGTTGCTGTGGTGAAGAAGGATAGTGGCTTCCAGATGAACCAGCT
TCGAGGCAAGAAGTCCTGCCACACGGGTCTAGGCAGGTCCGCTGGGTGGA
ACATCCCCATAGGCTTACTTTACTGTGACTTACCTGAGCCACGTAAACCT
CTTGAGAAAGCAGTGGCCAATTTCTTCTCGGGCAGCTGTGCCCCTTGTGC
GGATGGGACGGACTTCCCCCAGCTGTGTCAACTGTGTCCAGGGTGTGGCT
GCTCCACCCTTAACCAATACTTCGGCTACTCGGGAGCCTTCAAGTGTCTG
AAGGATGGTGCTGGGGATGTGGCCTTTGTCAAGCACTCGACTATATTTGA
GAACTTGGCAAACAAGGCTGACAGGGACCAGTATGAGCTGCTTTGCCTGG
ACAACACCCGGAAGCCGGTAGATGAATACAAGGACTGCCACTTGGCCCAG
GTCCCTTCTCATACCGTCGTGGCCCGAAGTATGGGCGGCAAGGAGGACTT
GATCTGGGAGCTTCTCAACCAGGCCCAGGAACATTTTGGCAAAGACAAAT
CAAAAGAATTCCAACTATTCAGCTCTCCTCATGGGAAGGACCTGCTGTTT
AAGGACTCTGCCCACGGGTTTTTAAAAGTCCCCCCCAGGATGGATGCCAA
GATGTACCTGGGCTATGAGTATGTCACTGCCATCCGGAATCTACGGGAAG
GCACATGCCCAGAAGCCCCAACAGATGAATGCAAGCCTGTGAAGTGGTGT
GCGCTGAGCCACCACGAGAGGCTCAAGTGTGATGAGTGGAGTGTTAACAG
TGTAGGGAAAATAGAGTGTGTATCAGCAGAGACCACCGAAGACTGCATCG
CCAAGATCATGAATGGAGAAGCTGATGCCATGAGCTTGGATGGAGGGTTT
GTCTACATAGCGGGCAAGTGTGGTCTGGTGCCTGTCTTGGCAGAAAACTA
CAATAAGAGCGATAATTGTGAGGATACACCAGAGGCAGGGTATTTTGCTG
TAGCAGTGGTGAAGAAATCAGCTTCTGACCTCACCTGGGACAATCTGAAA
GGCAAGAAGTCCTGCCATACGGCAGTTGGCAGAACCGCTGGCTGGAACAT
CCCCATGGGCCTGCTCTACAATAAGATCAACCACTGCAGATTTGATGAAT
TTTTCAGTGAAGGTTGTGCCCCTGGGTCTAAGAAAGACTCCAGTCTCTGT
AAGCTGTGTATGGGCTCAGGCCTAAACCTGTGTGAACCCAACAACAAAGA
GGGATACTACGGCTACACAGGCGCTTTCAGGTGTCTGGTTGAGAAGGGAG
ATGTGGCCTTTGTGAAACACCAGACTGTCCCACAGAACACTGGGGGAAAA
AACCCTGATCCATGGGCTAAGAATCTGAATGAAAAAGACTATGAGTTGCT
GTGCCTTGATGGTACCAGGAAACCTGTGGAGGAGTATGCGAACTGCCACC
TGGCCAGAGCCCCGAATCACGCTGTGGTCACACGGAAAGATAAGGAAGCT
TGCGTCCACAAGATATTACGTCAACAGCAGCACCTATTTGGAAGCAACGT
AACTGACTGCTCGGGCAACTTTTGTTTGTTCCGGTCGGAAACCAAGGACC
TTCTGTTCAGAGATGACACAGTATGTTTGGCCAAACTTCATGACAGAAAC
ACATATGAAAAATACTTAGGAGAAGAATATGTCAAGGCTGTTGGTAACCT
GAGAAAATGCTCCACCTCATCACTCCTGGAAGCCTGCACTTTCCGTAGAC
CTTAAAATCTCAGAGGTAGGGCTGCCACCAAGGTGAAGATGGGAACGCAG
ATGATCCATGAGTTTGCCCTGGTTTCACTGGCCCAAGTGGTTTGTGCTAA
CCACGTCTGTCTTCACAGCTCTGTGTTGCCATGTGTGCTGAACAAAAAAT
AAAAATTATTATTGATTTTATATTTCAAAAACTCCATTCTTTCCTAAATA
TTTTCAACAAAGGATTTCTTTATGCATTCTGCCTAAATACCTATGCAACT
GAGCCCTTCCTTCTCAGCTCAAGATTCGTCTGGTCTTTCCCTACAGCTTT
GTGTGTGCCATGGCCACATCTCCTGGGTACAGTTCAAGGAGACATCTTTT
CTAAAAGGGTCTGCGTGATCATTAAAATATAATCAAATGTAAAAAAAAAA
AAAAAAAA

Topbp1, or DNA topoisomerase 2-binding protein 1, is a protein that is required for DNA replication. Topbp1 plays a role in the rescue of stalled replication forks and checkpoint control. The Topbp1 cDNA sequence in Mus musculus is provided in GenBank Accession No. BC049797.1 (GI:29437357), incorporated herein by reference. The Topbp1 amino acid sequence in Mus musculus is provided in GenBank Accession No. AAH49797.1 (GI:29437358), incorporated herein by reference. The Topbp1 cDNA sequence in Homo sapiens is provided in GenBank Accession No. BC151237.1 (GI:153217452), incorporated herein by reference. The Topbp1 amino acid sequence in Homo sapiens is provided in GenBank Accession No. AAI51238.1 (GI:153217453), incorporated herein by reference.

An exemplary DNA sequence of the Topbp1 gene in Mus musculus strain C57BL/6J chromosome 9, GRCm38.p1 C57BL/6J is provided below.

NC_000075.6 (gi|372099101: 103305327-103350428):
(SEQ ID NO: 11)
CGCGCCCCCCGACTTGGTGCCCGTGGGCCCTCGGGCTCCACCTAGGTGAGCGAGCTTCGGCTCCTCCCGG
CCGGCGTGGCCGCCCGGCGCCTCTTGCGGCCTTCGCGCCTCTCCTAACCGGCGGGTGGGCCTCAGGTCCC
TGGGGAGCCGCGGCCTGGACGCTCCTTTCCCTGGGAGCTGGGCCGAGCGTGCACGACCTTGCACGGTGGC
TGCTTGCGTTCAGAGTTCGTTAACTTAGGAGCAAATCTTGCGGGCTGTGGGTACCCGTAGCCGCAGCGGC
GCCCTGCGTCGTCTTAAGCCCCATGCAAGTGAACAGTGAGTGACTGAGGACCAAACCAAGGGTCTCAGGG
GATTCAATCAGGAACGTTCATTTTGTTCTCACGTTTGCAAGGATCCGTGTCCTTTTTTTTCTTCTTCGAC
TTTGCCCACCTTCTGGGGCCCTTTGTAAACATCTTGAATGAATTGATGAACCAAGCCAGATAGCTATGTC
TTAGGCACTCTTGTCTGCATAAGAAAATAGTTGATTTGCAGGCCGGGCGGTAGTGGCACACTCCCTTTTT
TGCCAGCTTGAGTGAGGCAGAGGCCGAGCATCTCTTGAGTGCCAGGCAGACCTAGTCTACAGAGCGAATT
CCAGGACTGCCAGGGCTTGTCTCAAAACTAAAAAAAAAAAAAAAAAAAACCAAAAACCCTACAAAAATAG
TTGACTTGTACATTTAATTCCCTGATTGACTGACTGACTTTTGTTCTTCTTGACTGCCTTATTCTCACAG
TTCAGCTATGTCCAGAAATGACCAAGAGCCGTTTCTTGTGAAGTTTTTAAAGTCTTCGGACAATTCAGAA
TGCTTTTTTAAAGCTCTTGAGGTAAGAATTAATGGATATGTCTTATTTGATACTTGAGCTCATTTTCATG
TTTTAAATGTTTTTGCTTTGTTGGATTTTATGTCTTAGAAGTAGATAATTTTAATGAAATTTTGCTATAG
CATATGCTTAATACAGATAAATCACTGCTATATAATTTTTAGTTGATGCAAGTTTACCATTATATTTTTT
GGGAGGATTGAAATCTATTGTGACAATTGGATGTTAACCTACAGTTTGATCCCCTACCCCCGCCCGGTCT
GTGTCTGTTTGTGTGTGTGTCTGAGCCATCTTCCCAGTCCTCTGACTTAGAGTTCCTGACTTGTTTTGTT
TACTAACCCCATTGATCCTTTAAGCACTGAAAAACGGAGTCACAATGCAATAGTTTTGGTTCATTGCTTG
CTCTCTCTCTTTCTCTCTGTCTCTCTCTCTCTGTCTCTCATACACACACACACACACACACACACACACA
CACACACACACACACATATTCATGAATGAATGAATGATGGGAGTAGTTTTGAGCTTTGCCATTCACAAAC
CCTAGTAATGTCCTTGTAGCTTGGGAATCTATTAGAAATGCAGAGCCTTGGGATCCCAACCCACATTTGA
CAATTAGAATATGCATTTTAAACAAAAGTCCCAGTGCATATTCAGGTTTGAGAAGTTAGCTGTTTTTTTT
TTGTTTGTTTGTTTGTTTTGTTTTTTAAAGTTCACTCACTTAGCAATCATCAATTGAGTGTCCGTCATCT
GACAGGTTTTCCTCTAGCATAATACTTGGGGTACACCAATGATCAGAACAAGCAAATATCTCTCCTCTTT
CAGCATACATAGAACAGGAAATCGGTATTAATCAGGTGATAAGCAAGAAACTACACAGTATGGGGGCAGT
GAAAGAGTTGAGACTGAGTCTCACAGTGTAGCCCAGACTGATCTGGAACTCTGTGCTTCTGCCTCAGTTT
CCTGCATGCTGAGATTACAGGTGTGTGCCCCAAAACCTAGTACCTATGTAATGTGTTATAAGGGGTCTAG
TACTGTGGGAAAATGAAAAAGAAAAAGCCAAAACAATTTAAGGTTTTCCAGAGGATGTTTGTTGGAGGAG
AAAGACATGGAAGGGTTACAGCTTAAAACTGAATTTAAAAAAAAGCAAACTATTTCTCTTAAGATTATAG
ACTAGGGTCTTGTATAAGGTACTAGGTATCCGGATATGCTTATAAACAAAATACATCATGGAATGTTCAT
TTGTGTTGGTGTGTGGGGATGGTGTTTATGTGGGTGCTCTTGTGCACATGCATTCGGAATAGTATCTGCA
TACCACAGTACATGTGTAGATGTCAGAGGACAACTTGAAGCAGTTGGTTCTCCTTTTACCCTGAGGTTCT
CAGGCTGAATGGCAGGCACTTTTACCTGATGAGCCATTTAACAGAACCAACACATTCCTTCTCACAGGGA
AGAAATAAAACTATGTACATTTACAAAAAGAACCATTTTTTTTTCCTTCATAAAATGTAGTCAAGTGGTA
GAGGGAGTGTAGTTAGAAAGCAGGATGACCATGGGAATATTGCTTAGTGGTAGTTGTATAGTGTGCATAA
AAACCTGGTTTTAGTTCCCGGTGCTGCAAAGCAAACCCACCGTGGTATCTAGAAAGATCTAGACAGGTAT
CTCTAGGAAGTGACAGTGAAGCAGGCGTTATTGCAGTGAAGAACCACTTCCAATGAAAGCTTAAGAAAAG
AGTAGCTCCTGTAGTAGGACCAGAAGCAGAACCGCTTAGTAGGTCATTAGTATCTTCCAGGTGTACGGTT
TGGGGATGACTGGAATTAGCCAGGAAGGTGGATGTAAAGATGAGACTTGAGATAAGTGAGGGCTAAATTT
TATGATTTTGTAGTCATGAAGTTGTATGGTGCTGATGCATCTTTAATCCCAGCTCTTGGGAGGCAGAGAG
ATCTCTATGAAATGGAGGCCAGCCTGGACTACGGAGTTAAGTTCCAGGACAACCAAGGCTACACAAAGAA
ACCCTGAATGGGGGGCGGGGGGGGGGGGGGAAGAATTGGGACTGATCCATGTGCCCTTTCTTTTTGTCCA
GTGGTTCATTTCTGCTATATTCCTCAGTTTCTCTCCATCTTATTTTTTTGAGATAAATTTCTCACTGTTG
TAGAGCCCACTGATTTGCTAGACTGGTTGGCCAGGGATCTGGTGGGCTCCAGTCTCTGGCACCCACCCCA
CTGCCCAGTATTGGGATTGTGAGTCCATGCCTCCACACTAGGCTTTTTACATGGGGCCAAACTCAGATGC
CCAAGCTTACACTGCAGGCATTTTACCCACTGAACCCTCTGCTCAGCTCCTGGGTGGTTTCTTTAAAAGG
ATTACTAACTGTGAAGAATAGAGAGGAGACCAGTTCAGAGGCTTCTGCATGGGTCTAGGTGAGAAGCATG
CATGGGTCTAGGTGAGAAGCTTTATTTAATAAGGAAAGGGCTGGGTTTGAAATTAGCTCTGAGCACTTAG
TAGGACTTAAATTTAGGGTACTAATTTCTACTACAATCTTACTTGTTTTTTCTTTCAGTCAATAAAGGAA
TTACAATCAGAAGATTATCTTCAGATTATTACAGATGAGGAAGCATTGAAGATAAGAGAGAATGACAAAT
CGCTTTATATCTGTGACCGTTTCAGTGGCACTGTTTTTGATCACCTTAAACAGGTTTGCTGTTTCTAGAA
TACTTATTTGCTCTTTCACCCTCTCTTTTAATCTGTGTGTACCTGTGCTTGTGTGTGATGTGTCTGTCTG
TCTGTGTGTGTGTACATGCATGCGTGTGTAGGGTCTCATTCTTCATGAAAACTATCATGAGTGCTACGAG
TACCTTGGGATTCTTTGGGCTTGGGGTTCTTAGATATCTGAGCTGCTTTATTGGCTGTTGCTTTTTTGAA
AAGCATACTTGAATGTTTCTCAGTTGTTATAGCATAAGATTATTGCATTAAGATTGTATTTCTTGGGAAA
GGTGCCCTAGTGGTTCAGGGCACGTACTTCAAGGGATCTGAATTTGATTCACAGCACCCATGTCAGGTGG
CTCATGACTGCTTATAATGCCGGTTCCAGTACCTCCAACTCCAGGTTTATCTGTACAAAATGTGTGTATA
CCTACATAAGGATACATACATACATACATACATACATCATATAATTTAAAATAATAAAAAGTTTTATGTT
GTAAGAGTTTTTACCTGCATGCATGTTGGTGCACCATGTGCACACTTGATACGCTTGGGGGTCAGAAGAG
GGCATTGGATCCCTTAGAATTGAAGTTACAGGTTGTAAGCTACTGACTAAGGTACTGGGAACCAAACCTA
GGTCCTTTGTAAGAACAATAAGTGCTCTTAACTTTTTAAAATTAATAATGATAATAATAAGACAAGGAGG
AAAGTTTGTTTTAAGTGGTTGAAGTTTTGGAGGCAGTTATGAGCGCTCTTGTGGTTTTGCTTTGGAGGGT
GCGTGTCTATTTAGTGCTTGCTTGAGAAATTATGTGATAGTCTTTTTCTCCTAACATTTCATGGTTTTCT
GCAGTAGCTGTTAGCTTCTGTGAGTAACTAATAAAATGTCCCAAGTTTAATTTTGTGATTTTGTCCCAAA
CTTTTTACAGCTTGGCTGCAGGATTGTTGGTCCTCAAGTGGTCACATTTTGTATGCGCCACCAGCAATGT
GTCCCAAGAGCTGAGCATCCAGTCTACAACATGATAATGTCTGACGTAACTGTATCCTGTACAAGCCTGG
ATAAAGACAAACGGGTAAGCACTGAAAGCAGCCGGGCTGCCCCCACCTGACTTACCCAGGGCTCAACAGC
AGCAGGCCTCACACGTGGGGTTCACAGCTACAGGCCTCACACGTGGGGTTCACAGCCAGCAGACTTGTGC
ATGGCTCCCAGTGGTATTTTTTTCTAAAGATTATTATATAATTCCATCATTTCCTCATCCCTTTTCCTCC
CTCCAAATCCTCTCATACACCATTCCTTGCTTTTTTTTTCAAGTTTTCAAATTTATAATCTTTTTTTCAT
TGTTATATGCATAAATATATACATAATCTTAGATAAATACAACCCGCTTAGTCTGCATAATGTTACTTAT
ATGTATTGGTGTGCTCTTCCCTGGGGAAAGTGGAATATGATTGTAGAGACACTGGTTAAGTGCTTTGTTT
TGATTTAAAAGAAAAGTTACTTTTTTTTCTACAGTCAGTGAGTTTTGTTTTAAATCATTTAATTTTTGCA
GAACAGAAAATCTTTCAAGAAAGGATTCTAATCTGGGTGTTGTAGTGCATGCCTTTAATCCCCAACTCTT
GGAAGGTAGAGAAAGGCAGATTAGTGAATTCCAGATCAAATAGGGCTACATATTAATAAGACCTGTCTCA
AAAAAAAAAAAGGAAAGGAAAGGAAAGAAAAAAGAAAAGTGGCTGGCACCTTTAATCCCAGCACTGGAGA
GACAGAGGCAAGTGTGTCTAAGTTCAAGGTCAGCATGGTCTAAGGCCAGGATAGCTAAGGCTACACAGAG
ATGCCCTGTCTCGGGGAAAAAAGAAATAATCAGAAGCAACATAGTTTTGAATTATGGAATTTAAAAGAAT
AAATAAGGTCACTTATTAATGATAATAATTCTACCTGAAAGTGTTTCTGTAGGTGACTGAGGGTGCTTAA
ACTTAAGAAGGCTATGATGTCATTAGCAATATTAATCTGCAAGACCACATTGTTATAAATGTGGTTGATT
AGAAAGTATTGTTAAAAAGTATGTGACTGTTCAAGAAAACAGTTCTTTTCAGGTTTTTTGTTTTTTTGTT
TTTAGGGGAAGGCTGTGGATTGAGCCCAGAGCCTTGTGCATGGTAGGCAAGCATTCTACTACTGAGCTAC
ATCACCAGCCTACAAATAGCGATTTTTAAATTTAAAATTGATACACATCTTTCCAAAAATAATTACTTAT
TTTTTTTAGTAGCAAAAAACATTTGTATGTTTAGTCTATCAACAGTAAATGATACTTATGGGGGCTGGAG
AGATGGCTCAGCGGGTAAGAGCACTTATTGCTCTTAGAGAGGACCTGGGTTTGGTTCCCAGTACCTGCTA
GATAGCTCACAACCATTTAAACCTTCAGTTTCAGGGGGTCTATCTGATGCCCTCTTGTGATCTCCAAGGC
ATGCAAATAATGTATAGACATATACTCAGGCGAAAGAAACACTCATACACATAAATTTTTTTAAAAAAAT
TACGTGATTTTTTTTTTTTTAAAATAGAAGAATTGTTGTTTTCTCCAGCATTAACATATTTTCTTTTGGG
TCTATAGGAAGAAGTTCATAAATACGTACAAATGATGGGTGGACGAGTATACAGAGACCTTAATGTATCA
GTCACTCACCTTATTGCAGGAGAAGTTGGGAGCAAAAAGTACTTAGTTGCTGCTAATCTGAAGAAGCCTA
TTTTGCTACCTTCTTGGATAAAAACCCTTTGGGAGAAGTCACAAGAAAAGTGAGGCACATTCATATGCTT
TGTCTGTTTGAAAAAATGATGCATTTGGATAGTTTTGACGGTGTTTGGTTTTCATGGGGAAGCTTTATGG
TATCTCTGGACTTGTTACTGAGAAACAAAACAGGGTCTTTGTTCATTTCTCCAGTGACAGTAGGGGAGAT
GGTCCATAGATTCAGCAGTGCCTAGAGGGCAAATGCTTTGATAATGAAGTTCTTTTCTGCATCAAATACA
TCTAAAATAATTCAGTTTGGCTTTTGTGTTGCACTGTGTAATACTGTTCCCCAGTATTAAGATGGAGAAT
AAAGTACCTACTTGGGCAGCTCTGTTCCCTGCTGGGTAGGGTGGAGGTGCAGAGAGGGGTAGAGGTAAGA
TTAATATACATATATGTAAGCCATCTGCTGAGAGGTGATCATTCCGTGTACATCGAGCCTATTTTCTGCA
CTGGGCTGGACATTAGATGTATGTAGAGATCAGAACAGGATAACAGTTTACCTTATGGAGTTTGGAGGTA
CACTTCTGTTTTCCCTAAACTTGGGTTTATAGTAATAAATCCGGGTATCTGAATTATATCTGCATTCTGT
GGCATTTTTCTGCACTTATTTCAACATTATTCAGCATGTACTCAGTACATTTCCTAGTTAGTACCAGCCT
GGTGAAGTTTAGCACACCTACTTATTGTGGCAACAAAATGGCAGTACTATTGTTGGAACTCTGCCGAAAA
GCATTCCTTTTACTTATCAAGAGTAATGGAAGAATGTGTCTGTTTTATACCAGACAACCTGTGTAAACGT
TAGGTTTTTAAGTACAGATCCAGTCTTATAATTCTGCCAAATGCTCCCCATTTATTAGCAAGGACTTAGG
AGTTAATAGTGCTTTCTTTAAGATAGTCTTGGGACTGTTGCTGAAATACAAGTTTTTATTAGAGACTTGG
GACTGAGCTAGATGAGCAGTTAGTGTAATCCTCAATGTCAGTTTTCCTTTCTGAAAAATGTTTATAGATC
CTAAAGAATAGCAACTGAGCAGACAGGGAGAGCAGTAGGAGCTAGCACGGTCTGAGTACTCGGGCTCAGT
TGTCTCCTCTTCTTACTTTCTCCAGAAAAATAACCAAGTACACTGATGTAAACATGGAAGACTTTAAGTG
TCCTATTTTTCTTGGTTGCATTATCTGTGTGACTGGCTTAAATGGAATACACAGAAAGACAGTCCAGCAA
CTCACGGCTAAGCACGGAGGCCAGTACATGGGACAGTTGAAGATGAATGAGTGTACACACCTCATTGTGC
AAGAACCAAAAGGTAAACAATGCCTTAGATGTGGTTGAGCGTGCAAGTCCTGTTTAAGCACTAATTCTCT
GTAGACCACCTTGTGTTCTGATGTGTCATTGCCTTAGTTTATCTGGGTTTTTTTTTGCCTTTTAAATTCT
TGAGAAGATAGTTTGGGTCACAGTCTAGAAAAATTAAGGAACTGTCCTTGTTATTGTCCAGAATATGGAA
TAAACATAAAATATCAAAAGTTGCCCATGATGTTTCACTACCTGACTTAGACCTTGATGAAAAAAAAACA
AAACTCTTCACAGCATAACAAATATGTATAGCTGAGTGGTATTCTTTCTCATGACTAACTTCAGAAGCTT
AGTTAAGCACTTAGTGGGATGAATTGGTTTATATTACTGCATTTGGATTGGGGTGCGGGTGGGTGGTATG
GGTAGAGTTCCCGAGTGCTGGGCAAGTGCTCTACCACATTTTCCAACTAGTCTATGAACTAGAATTTCTT
TCTTTCTTTCTTTCTTTCTTTCTTTCTTTCTTTCTCTTTTTCTTCTTTTTCTGAGACAGGGTTTCTCTGT
GTAGCCCTGGCTGTCCTGTAACTCACTCTGTAAAGTTTTACTATTGAGAAACTTTAAAATTATTACTGTT
TTTGATATTTCGGGAATTTTATTCTAGGGAATAGTTTTGCTAATACTCCTAAAATCAGGGTTAGGGTTTA
TACAAATATATAAGAAGAGTCTTGCTGTATTTTTAGTAACTCCTTTGTGTTTGTACTGTTTTACTAAGCT
ACCTTGATAAGAAGCATTTGAGAGTGATTGTTAGTTACTTGATAATTGTCACTTGATCAGGAGATTGGTG
TAATGGTTATGATAGGACAGTTACTGTGGCTTTTCTCAATCTAAAGGCAGTCTGTTTATCTATTAACTAT
GTTTTAAGGTATGCGGGATTGAGCCTAGGTCTTGCTTATGCTAGGCAAGTGCTTTCCCACTGAGCAATGA
CCCTAGTCCTGCTTTCTGTTGGAGACAGGCTCTCGTCAAGTTTGCCATGCTGCTGTGAGATGTATGATTT
TCCTGTCTTACAGAATATAGACTCTACCAGGCCCTGCTCCCTTGACTTTATGGAGTACCAAAAGTTCTCC
ATAGAGCGGGGTGTCAAAGGAGGAGGAGGAGGCAGGAAATCAGGGCTACACATCTTTGTCTAGTCAAAGA
TAGTATTTATTACGTTGCATACATTTATGGTATCTGCAGTTGTATTATATGTTCTTATTAAACAACATTA
ATAGCCGGGCAGTGGTGGCGCACGCCTTTAATCCCAGCACTTGGGAGGCAGAGGCAGGCGGATTTCTGAG
TTCGAGGTCAGCCTGGTCTACAAAGTGAGTTCCAGGACAGCCAGGGCTATACAGAGAAACCCTTTCTCAA
AAAACCAAAAAACAGAAACAAACCAAAAAAACAACATTAATAATGTCAAGCATGATGGTGTCTTCAGTCC
CAGTGCTTGGGAGGCATAGGCAGGCAGATCTGTGTGAGTATAAGTCCAACCTAGTCTATCCAGGCTAACT
AAGCCTACAAATGAGACCCTATTCCAAAGCTGGTTGGCTGGAAACTTGACTCAGTGGTTAAGAGCACTGG
CTACTCTTTCAGAGGACCCTAGCTTGATTCCCAGCACTTATATGGCAGCTCACAACTCTCTGTAGTTCCA
GTTCCAGGGTGTCCAACACCCTCTTCTAACCTCCTTAAGCATCAGACATGCAAGTAGTACACAGACATAC
ATGCAGACAAAACACCCAGACACATTAAATAAGAAATAAAAAATTCGCTGGGCGTGGTGGCACACGCCTT
TAATCCCAGCACTCGGGAGGCAGAGGCAGGTGGATTTCTGAGTTCGAGGCCAGCCTGGTCTACAGAGTGA
GTTCCAGGACAGCCAGGGCTACACAGAGAAACCCTGTCTCGAAAAAAAAAAAAAAAAAAGAAAGAAAGAA
AAAATTCTTAAAGGTAAATATATTTACTGGTAAGCTTTAATCATTACTTTTGTTTGGAGTGACTTTTAAA
AAAATAATTTACTATGAACTAACTATATAGTCTATGTATTTCTGTATTTCTTTCGTAATGAACACAGGAC
AAAAATACGAATGTGCCAGGAGATGGAATGTGCACTGTGTGACCCTGCAATGGTTTCATGACAGTATTGA
GAAAGGCTTTTGTCAGGATGAATCTATATACAAGGCTGAAACCAGAGTAGAAGCAAAGATGGTGCCTGAT
ACTTCAACACCCACTGCTCAGAGCAATGCTGAAAGTAAGTCTCTTTGAGATTAAAATAAACAATTACTTG
TTCACATGATAATTGATTGACTGGGGATGTAGCTTAGTGGGAAAGCTTTTGTCTTGCAAGCCCAGGAACT
CAGTCTTAAACACCAAGGAAAAAGTGAAGGAGTTGATTTGTTAACAGGACCATAGGAATATAGTAGGGAC
TTTATTTTCTCCCTTTGCCCCTGGGTAGAATTAAATAAAATAGCCATGCGTAGACAGCTTTCCAGAACTT
CTTTTGTATGCCTTTTGGAATGAGAGATTGCTTTCTTTCACATCCTAATTCACACAAGAGGGGATTAAGA
ACACTAGTGTATGGGCTTAAGGGAAGAGTTGTTCGCCCCTCTCCACATTTGTTTGCTTGGTTAATTGGTT
TTTCAAGACAGGGTTTCTTTGTATAGCTCTAGCTGTCCTGAAACTCCCTCTGTAGATTGCTGAGCCCGGC
ATGGCCCTGGTGGGGACAGTTCTTGCCCCTGGGACAAGACCCGGTGTGGGCCTCCTCAAGTGTTGATGGC
TGGTGGGTAGAAGACTTGTTGTGTAGTAATGTACGTATTTTCTGGACCAGCAGTGGGCGAGTCTTTCCTT
CTGAAGAATGTCCTCCCTGTTAACTTTAATGATTCCATGATAATTAGAAATAGAGTCCAGGAGAGTGTGG
CTGTGTCTCGGACCTTCAGGACAGCCCTTAAGGATATGGGAAAGAACTCTAAATACGTGAGTTCAAAAAT
GTAAGGATGCAATATGAATTATGAGGGGCTTCACGAATCTAAAGGAACAGAGATAGCTACACTATGAGCC
TGCTTGTCAGAAAGATACAAAGGCAGGCGGATACAAATGTCCCAGGCAGATTCCTGGGACACAGCAAGAT
TAGGTCCAAGTGTGGTTGAAATGATAATTTCCAGTTCTACCTAGCTAGTTTAGCATCTGTGTGTAAGAAA
CTAGCAGATCTCTGAATTCTTTTGCAATGTTAAAAGAAAATGTGTTTGTTGTCTCCTAAGAAACAAAAAA
GTCACTGATGCTGATTCATAGGATAATTAAAAAAAGGAACCTGGAATAAATAACTGAATTGTTATGTAAA
TAAAAAACTGGGCTTTTGGTCTACCTGATGTGAACTAGCAGAAAGCAGTAGCAGATCCTTTTTAGAATTT
TCTCTAGTGAGAGCTCTGGAGAACAAAAACATCTCTGCAAGAATTTTTCTTAATTTTCCAACTTGGTAGG
AAGATAAAATAATTTTTTTATAGCAGTTTTTCTGACTATGGTCAGAAAACCCATGAGCTATCCAGATAGC
TTTTAGAATTTTTCCTAGCAGAGAGAACTGTCTCAACCAGAGTTTTTAGAATAGTGAGAAAAGGCTGTCC
AGAGCAGAGCAGAGCACATACACACTCATACACACACACACACACACACACACACACACACACACACACA
CACACACACACACTAACTCTCACACACTCACACACTCAGACTGAGAGATCGAGCAGTCTGGAAAGCCATC
TCAGCAAAACCTAGGCTGCCAGCTTGGACCCACAATTAGACTTCGAGTTGTTCTTTTCGCTGCTCTCAGA
CACTACTTCTCTCAGAACCCCTCTCTGCCAAGGCTAGTCCCTGGCAGTAGATCATTGAGCTGGCCTCAGA
TTGAGATTGCGTCCACTTTCTGAGCACTGGGATTAAAGGCGTGTCCACCATGCCCAGCCTATATTTTCTG
TATTGGTTTACTGTAATGATTATAAATCATTACATAAAAATTCGGCCTGCATGAAGTCCTGAGTTTGATT
ACCTTTTGATTACTACTACTGCATTATAAAGCTGGATGTGGTGGCACATACCTATAATACCAGCACTTGA
GAGTTGGAGATGGAGGGATCAGAAGTTTAAAGTCTTCTTTGGCTACAAAGAGTTCAAAGCCAACCTGGAT
TTGGTGAGACTCTGCCTCAGAAAACAAAACAAAAGGCCAGGGACAGTGGTATATAACCTCAGTGCCAGCA
CTGCGGGCTGAGGCAAGTTCGATGTGGTCCTGATCTGGGTAGTGCAGTCCTTTTCAGCCTTCCTAATGCT
GTGACCCTTTATTACAGTTCCTCGAATTATGGTGACCCCGACCATAAAATAATTTTGTTGCTACTCTATA
ACTGTAATTTTGCTACTGTTATGAATTGTAATATAAATATCTGTTTTCTGGAGGTCTTAGGTGACATCTG
TGAAAGAGTTGTTTGACCCCCAAAGGGCCTCAACCCACAGATTGAGAACCACATGCCCCTACATCCACAT
GCCACTCTGATGTGTCAGCTAGGCTAGCATATTCAGTCAATACTGAGGTCTTATTTCGGAAGTTCCATTT
AGCAAAGTCTTCCTCCTTGGACCTTAAAGCCAGCCTTATGGGCTATAAGCCACTTTAAGGAAATATTAAA
TATAATTTCACCTAAAGAATGAAACTTTATAGCCGGGCATGGTGGCGCGCGCCTTTAATCCCAGCACTCG
GGAGGCAGAGGCAGACAGATTTCTGAGTTCGAGGCCAGCCTGGTCTACAGAGTGAGTTCCAGGACAGCTA
GGGCTACAGAGAAAACCTGTCTCAAAAAACCAAAAAAAAAAAAAAAAAAAAAAGGATGAAACTTTTATTT
TTTTTTTCAGTAAAAATCTCTGAATTTTTGTAGGTTTATGCCTTAAGCCCTTTCTAGGAAAAGTTAGTTT
ATTAGGAAGCAGGTAATGGCTCTTTTACCTCAGGAATTTTCATTTCCTTTCACATTTTAGGTCACACGCT
CGCAGATGTCAGCCATATATCCAACATAAATGGCAGCTGTGTAAATGAAACAATGTTTGGTTCAACTACT
AGCAAACTTGAATGTTCCCTTGAAAACCTAGAAAATCTGGACATCAGTATGTTTCAAGCTCCTGAAGACT
TATTAGATGGCTGTCGGGTATGTCTTTATTATGTAACAAAGTCAGAAGGACAAATAAGAGACCTGAGTTT
AGGGCTAGCTTGGGCTACACAGGTCCAGACATCCATACACCCTCAGCCTGACTCCAAAGACCCCTCACAG
ATGGACTTGCTCCTTATGTGCGTAAGCAAACCTTGCACTAAATTAGGAAGCTGACACTTGGCACACACAC
GCTGTGCCTGTGGGTCTGTCTGGGCTGTAGCTTCATCCCTTCTATTGCTGAGTTCTTAAGTCTGCTCTGA
AACCTTACCGCCTACTTTACAGATGTTCATCCTTATCATTTGTTTACAATACATTAATGACGGTGATCAT
TTCATGTTACATTATGTTATTTAAAATATAACTTAATCCCCATGAGTAAAGTGGTTATTATCTTGAAGCT
AATACTTAGATGATTAAAATCCATTTTTAGGGCACTTAGGCTAACGTGCTTGTTGCTAAGCCTGACAGCC
TGTGCTCAGTCCCTAGCACCCCATGTGGTCGAAGGAATGAACGTAATCCTGTCCTGTGACATGTACTTGC
GCCACGCCCACACGTGTATATGTACAAGACTTAAATACAATAATGTGCTTTCTCAGAGTCCTAGACAGAT
AATACTGTGGTCTTAAAGTCTGCCTTCTTAATTGTGTACAAGACCTTGGGTTCAGTGCTCAGAAATAAAA
AGAGAAAAAGATTAGATTTGTAAGATTATTTTCAGTGTGTGCATGGAAGCTCCGTGATGGAAGTGTCGCG
TATTTAGACCCACACACCCCACACAGTGCTAGGAGGGGAGTGAGAAGCTACACATTTTTCCTCAGAGTAG
GCAGTTAGGTTTATTGTTAAGGCCACATATCTGTGATCGCTCCTAAGATTTATCAACTTACTGGTGGGAT
AGAACGACACTTTGTGTAATCTTTTTTCTTTGATCTTGGGACAGACTAGTTCTAGATGTAACTTTGAATT
TTTTTTTTTCTATGTAGGAAGTATTTGATTCCTAATTTTTGCTAGGATGTATTCTAGTAATGGTGATAGT
TTGGGACATGTGGAAATAGATTGTCCTTTTTGAAATTTTTTAAAGACTTGAAATTTTTAAAAGACTTGAA
AAGTAATGTCGGCTATCCCTCACATCTACAACCCCTGTACACATGTTTCTTCAAAGCATTGGGCTCAGCT
CCTGGATTTGTTGTTTTGGTTAATTCAAGTCGAAAAGAAATTTCAGGAAAGATAACTATGTAGGAAGGAG
TTGGCTCCGAGCCTTGAGGAGTTTGCTCAGTTCATATGGTTGGTCAGTACCAACTGAAAACATGCAAGCC
TGGGTGCTCCTGAGTTTACAGTGAACTGCAGTGTTTCTCTGTAATTATTCATCTACAGGCAGTCACTCCT
TTACAATTTAAGGAGCTATTTGTATGTTAGAAATAGCTCTGCCACCCACCAAACCTGATGACCTGAGTTC
CATTCCTGGGACCATAAGGTAGGAGAGAACTGAGTCCCTCATGTTTTCCTCTGGCCTCCATATATGTATT
CTGACACCTGTGCATGTGCACACATATGTACACACACACACACATATACACACAGTGAAAATCTTATAGC
AAGCTGTGCACCATCATTTAGAAGATGTTGCAGTTTGTCCTGTAATTCATCTGCGGCCTCAACCCTCCTT
CAGGTTTTTACTGAGAGATGACAGTGCTGTGGTTAAAATACCAGTTTTAGGCTGAGGAGATGGGTGAGTG
GGGCAGGCACTTGCTGTGAAAACATGGGGGCTGAAATTTGATTCCCAGCACCTATGTAAAAGCTGTGAGT
GATGGTGTCCAGCAGTGGGGGGTGGAGGGGAGCTAGCTGCTAGTGTGTGTGCACACATTAGATGTAGACT
ATAGTTGACTCCATGTGTATCCCAAAATGTACATGAAGTATCTTGTTTACACACAGTGGATACAGTTTTC
CTTTAAATTTGTTTCTGTTGAGGAAAAAAATAACTTGAGTATGTTAATCCAAAGCAAATGTTTTCTTTGC
AGATATATCTTTGTGGTTTTAGTGGCAGAAAGCTGGATAAGCTGAGAAGGCTTATTAACAGTGGAGGTGG
AGTTCGATTTAATCAACTAAATGAAGATGTAACTCATGTAATTGTGGGAGATTATGATGACGACGTGAGG
CAGTTCTGGAGCAAATCATCTCACAGGTCCTGTCAGTCTTCAGCTCAGAGCACTTGGTACAGGGATGGTT
TGGGTTAGCTTTGCATGTCTGTGCGTGGAGCTGTGGGAATGATGGCCAGTGGAGTGACGATCTTCTACTC
GGGAGCCTAACTCTGCTCTTTTTATAGGCCTCACGTAGTGGGAGCAAAGTGGTTGCTGGAGTGTTTTACT
AAAGGATATATACTTCCTGAAGAGTCCTATATCCACACTAACTACCAGCCAGCTGGAATTGCAGTTTCCG
ATCAGCCTGGAAACCAGACAGCTGTCTTAGACAAGAGCGGCTCATTCTCCAAGAGCGCCCTTGTTCCTGC
TGAGAGGCTTCAGCAGGCCGACGAGGATCTGCTTGCTCAGTATGGAAATGACGACTCCACGATGGGTGAG
GAAGAGTTGATTAGCTTTTGTAGTTAAGAAGCAATAGATAATGCATGATAGCTGCCATATTTTTATGAAC
ATTTTATTTTCAAAAACTTTTAGATTTGTAACTTTGTAACCAGGCAAGGCCATCAGGCTCCAAATGATTG
AATCATGCTCTCAGTAGCTCTATGTTGAGTATGAACTGTTTTTGTTTGGGGTGTGTGTTTGTGTGTGTGT
TGGTGCCAGAGGTCAGAGGTGAGACCCTCATGGAGCTAGAGTTACAGGCATTTGTGTCTAATGTGGGTGC
TAGGAATAGAACTCGCGTACTTTGTAAGAACTGTGCATCCTTTTAACCTCTGAGCTATCTCTCTGGCACC
TCTGTGTGAATTGTAATGGTAGAAAACAAAGGCCCTCTGACTGGGCTGTCGGAAGTCTTTCCTGCATGCA
ATACTGTTCCTGGTACTGCTGTCTCAGCGTATAGTTTCATCGGTGTAGTAAGGTAAATTACCAACAAAGA
GTTAGAACTAACCTTTTTAGAGTGTATTATTTGATACATCAAAAGATAGGGTTTTGTGGTGACTGTCACG
TACTGTTTGTTTATGAAAATAGAGCATACCCCTGTGCCAAGAGCATTCCTGTGAAGTCCTCCTGTTTTTC
GCTTTCTAGTTTGTGTCCTGTGTAGGTTTATAGATGAGCCTTTGGCCTTCGTGGCTAGGTTGTTTAGTTG
TTTACATTGCTTATTGCAATGCTGAGGCTGACTCAGGCGTAGGCTCTATCACTGAGCTGTTACTCTTTTT
ATTTTTAATTTTAAGGCTTAGTCTGCTCTTGGACTTCCTCCGTGAGGCAGGTAGATTTTGAACTTGGAGT
CTTTCTGCCTCAGCTTTCTGAGTAGTTGGTTTTTCAGGCCTCTTCTACTAGGGCTGGCTTCAATTTACAT
AAAGTATATGTACTTTGTAGTTGTAAAGACATAGTTTTCAGTTCTTTTTTTTTTAAGATTTTATTTCTTT
TAAAAAAAGATTTTATTTATTTTACGTATATGAGTACTATAACTGTACAGATGGTTGTGAGCCTTCATGT
GGTTATAGGAATTGAATTTAGGACTTATGTTCGCTCAGTTCAACCCTGCTTGCTCTGGCCCAAAGATTTA
TTATTATATCTAAGTACACTGTAGCTGACAGCTGACTTCAGATGCACCAGAAGAGGGCATCAGATCTCAT
TACGGATGGTTGTGAGCCACCATATGGTTGCTGGGATTTGAACTCAGGATTTTTGGAAGAGCAGTTAGTA
GTGCTCTTACATACTGAGCCATCTCATCAGTCCCCAGTTTTCAGTTCTTTTTGTATGTGTGTGGTTTTTG
TTTTTTGTTTTTTTTTGTTGTTGTTGTTTTTCGAGACAGGGTTTCTCTGTGTAACCCTGGCTATCCTAGA
ACTCACTTTGTAGACCAGGCTGGCCTTGAACTCAGAAATCCATTTACCTCTGCCTCCCAAGTGCTGGGAT
TAAAGGTGTGTGCCACCACTGCCCAGCCAGTTTTCAGTTCTTATTGAATTTTAATTGTTTAATTGTGGAA
AACAAATTTTTTTATTATTTTTCTAAATCTATTCAGTGGTACACATTTGAGTTTCAGCACTTGCGAAGCA
GAAAGAGGCAGAAGGAGCTTGAGGCCATCCTGGGCTACATAACAAGCTGCAGACCAGCCAGGACTAGAGA
GACCGTGTCTCAAAAACAAAACAAAGAAACAAAACAAAGCATGTTTACTAGACGGTGATTTTTCAGGAAA
TTTGGTTTTCTACTCTGGTTTCATACTTAGAATAGACATGACAGTTGTTCTCTGATTGACGATGGTGATA
CTTAGCATTTGGGATAAACCTGAAGATGGAATGAATACAGAGTCTCCTTTCACTGTATATTTAAACCTAA
GTTTTATAAGTATTAAAATTATCAGACTCTTACATTCATGTCTTTGCTAGAAAATCACTGTGTAGCTTTG
GTTGATTTGGATATTGCTGTGTAAACCAATTTGGCCTTGAACTGACAGAGATCCACCTGCTTCTGTTTCC
CAAGTATTGGGATTAAACCTGTGCATCACCACACCCTGTGCAAATAAACTCTTTTTTTTCTTTCTATTAT
TCACTGTACATCCCAATATCCATCCCTTTCCTCCCAGTCCCCTCCATAGCCCCTATTCCCCGTCCCCCAT
TCCCCCTCTCCTGTGAGATGGGGGACCCCCACCCTCCAATCATCAATCACCCCATCATGGCACATCAACT
CTCTGCTGCACTTGGTGCGTCTTCTCCCACTGAGGCCAGACAAGGCAACCCAGCTAGGGGGATGGGATCC
ACAGACAAGCAACAGATTCAGGACATCCCTGACTCCAGTTGTTAGGGGACCTGCATGAAGATCAAATAGC
ACATCTACTACATATGTTTGGGGAAGCCTGGGTCTAGCCCATGTTGGTGGATTTTTGGCTGGTGGGAAAT
AAACTTTGTTTTGTTTTTTCTCAAGACAGGGGGAAATAAACTCTTAAGTCTAGTAATTGTCAAATAGAAA
ACTTGCCTTTTTCTTTTCCAGTTGAAGCTAAGCTATCTGAAGCTTTAGAGCCTGAAGTCGGGCCTTGCCC
TGGTTCTGCTCACCGTGAGCCCTGTGATGATTCCACTCACATTTCTGTGCAAGAGGAAAACAAGTCGTCT
GTCAGTCATTGCATCCTTGATGACTCTACAGTTCGTGAAGAAGGCTTATTTAGCCAGAAGAGTTTCCTTG
TTTTGGGTTTTAGTGTTGAAAATAAATGTAATATTGTAGACATCATAAGAGAGCATGCTGGCAAGATTGT
GTCCCTTCCCAGCAGAATTGTTGCTGACTATGCTGTGGTTCCACTGCTGGGATGTGAGGTAGACGTGACG
GTGGGAGAAGTTGTTACAAACACGTGGCTGGTAAGAAGCTGTGGCAGTCGGAACCCTGTTTTGAAGGAGT
ATTGGCTGCCAGAGTCTTTATGTTTACTTTTGAGCCTGTTCTGTTTATCTAAATTGGCATTTGGTTTGGG
CAACGGCAATTTTAAAACATTAGCCTGGGAGAGCAGCAGGATGGCCCAGTGGGTAGAGGCCCGAGCTGTG
GGAGCCTGAGCACCCGAGCCTGAGCCCTAGACCCTGGTGGGAGAGAGAACTCCCACGAGTCCTCTGACCT
CCGTGCTGCAGACACTTTCTCTCCCTCCTTTTCCTTTTGCTGTGCCCCCTCAAATGCACCGGCTATACTT
CTGGCCTCAAACTCAGAAATCTGCCTGCCTCTGCCTGAGTGCTGGGGATTAAAGGCGTACACCACCACGC
CCAGCCCAAACCTGTATTTCTTGATCCTAATACTAACATCAAGAAGTTGCTATCACAGGGCTAAGGCACC
AGTATTCTTTAAAGTAACTTGCAATTGCTGTATTAATCCATCTTACTGATTTTGATATGCAAATGTGAAG
GATTGGTATCTGTATTTTTCTTCAAATATCATTTGGAATATGTCAAGAATGAGCATACTCAAGTCTATGT
AAAATTCCTTTTTCCTTAAACTAAAATTGCTACTATTATAACTTGAGCTTCTCTTAACCACTTGTTTCTG
ATAGTAGCTTTATTGCTTTTGTTTTCAAAGGTTACGTGTATAGACAACCAGACATTAGTTGATCCCAAGT
CGAATCCCCTCTTCACACCAGTCTCAGTAATGTCGGGAGTGACTCCTTTAGAGGACTGTGTGATTTCCTT
CAGTCAGTGTGTGGGAGCAGAAAGAGATTCCTTGGTATTCTTAGCAAATCACCTTGGAGCAAGGTATGTA
ATATTTTCAGTATATCTCCTGCCTGTGAGTGAGTGTGCATGCACACACACACACACACACACACACACAC
ACACACACACACACACACACACACACACACACACACACAGTGTTAGGCAATAAGCGGAGGGCCTTGTACA
TGCTAAGCAAGGGCTCAACTATGGCAACCCTAACTGCATCAAGTTTTGTTTGTTTTGGCTTACTGGAAAG
TGTATAATTACATTTGCCTAGGATTTTGTTTGTATATGTGGTTGATGGGTAAATACTGGGGCATACACAG
CTAGGTGGATATCTGGCAAACTTGCAAGGCTGTGGGCAGGCATTGTTTTTTTTTTTTTCCCCCATTGGCT
TAGTCACCATGAAAGGCTGTTACTAAATTGTGCTTCTCTAAGCACTGTATGTGCCCTTCAGTGCCTCCTC
CTCTTCCTTCTCTTCCCCCCTTCTTCCCCCCACCTCCTTTCTCTTTCTTATTTTAAGTGTATATCTATTG
CTTGTTTTTATCGAGAAATACAAATGGAGCACTTTTTTTTTTTTTTTTTTTTTTTTTGGTTTTTCGAGAC
AGGGTTTCTCTGTGTAGCCCTGGCTGTCCTGGAACTCACTTTGTAGACCAGGCTGGCCTCAAACTCAGAA
ATCCGTCTGCCTCTGCCTCCCGAGTGCTGGGATTAAAGGCATGCACCACCACACCCGGGGCAAATGGAGC
ACTTTGCAAGGAATGCAAGGAACACTTTGGAAATAAAGGAATTAAAAGTTTCCTATAAAATCCTCATTGG
TCATTACGTAGTAGCATTTGAAAAAGAGTAGAAATCAGCTGTATCAATGCATTTCCATTAACCAACCTCA
TATTTAATGGGGATATTTTTAGGTTAAGAGCATAATGAGCATAATGCTTTTAAGCAACACTCTGAACATT
CTGAGGGGTAGAGTCACAGCAGGGCTTCTCTGTGTAACCCTCTCTCCGTAGACCAGTTTGGCCTTGATCC
AACTCAGGTCGGCCGCTCTTGCCTCTAGAGTCCTGGGATTAAAGGCGTGCACTGCCACCACCCAGAGTAT
GTTTCTCTGACATTAACCATGCTATTATTTTTTTAAAATGAGCTAATTGTGTGTTCATTTGCTTTATTTC
CATGTAAAATTTTAGTGTTCAAGAATTCTTTGTTCGCAAAGCCAATGCAAAGAAAGGCATGCTCGCCAGC
ACACATCTTATAGTGAAGGAACCGACTGGTTCCAAATACGAAGCTGCAAAGAAATGGAGTTTGCCGGCAG
TTAACATTTCATGGCTCTTAGAAACTGCGAGAATCGGGAAGAGAGCAGATGAAAACCATTTTCTGGTTGA
CAACGCACCTAAACAAGGTTAGAAGTCCTTGTTTTTTTTTTATGTATTTTACAACTTGATGGTTTCTGAA
ATAGGGATGTTCCAGTACTTGCTTTAAAACATTTGTATGACCCTAACCTCAGTCAGTGGTGCTTACTTCA
GAACCCCTGAGTGAAACACGGAAAGCAGATCAATGAAGAAGCGCATCAGGGTCAACCCTCGATTAGTCCT
CAAGGCGAGTGACGAGAAGGTGACCCCCGAATGGCTGTTAGAAGCAGTTTTTATACTTTTTAGTGGCACA
GGTTACATCAGCAAGGTAACAGTTCATACTTATTGCCTAAGCATATTGACCTTAAAATCTATTGGCTAGC
AAGCATGACACACATTTGAACTCTACCTAGGACTTTCCAGGGGGTCCGATGACTATGAGTCAGTACGTTC
TGCTGTTTTTCTGAGAATTTTTTTACTCCAGGATGTTCCTGTGGGTGAATGGAACTTGGCCTAGCTTTGA
CCCCAGGTGAGAGGGGGAAGCTGTAAGGAGGGTGTGAGACTGGAAAAGCCCTTAGGGTCCCTCACAATCT
TTTATTTCTGCTATTGTTTATGGTAGGTCCTGTGAGTCTTGGTTGTTTGTCAGTGGTGCCATCTCTCAGC
TTAGTGTGTACTGTACCAATTGTTGTCAGTACGGCCTGAAAGGAAAGTGCACACACAGTCTAGTTCAGTG
CTGTGCAAGGCGTGAGCCTCTATCCCAGTTAGTGTTAATTCCTTTTATGGCTTGCGCTAGCATGCTGGGT
AGCTAATGTGCTCTTGCTGCTTATCTGGGTTTTTCGTATGTTCCCACAAGATAACTTACGACTTGGCTAC
TGTCCTAGTTACTTTTCTATTGCTGTGATAAGGAACCATGGCCAAAAGCAGCTCGGGAAGGGAAGCATTT
ATTCCAGCTTACAGTCTGTAGTCTGTCACTCAGGTAAGCCTGGAAACTGATGCGGAGGCCATGGACGAGT
GCCGCTCACTGGCTTATTCCTTATGGCTTGCTCAGCTTGTTTTCCTTTACCATCCAGGGACCAACTGCCC
AATAACGTACTGCCCATAGTGAACTGGGCCTCCCAAATCAATCATCAATCAAGGAAACATGCACAGGCCA
ATCTGGTGGGCGTACTCTCAGTTAAGGCTCCCTTTTCCCGAGCCTGTGGCTGGTTGTCGTAAAACCAGCC
AGCACAGCTATTTTCCTTTGTCTTGAGTTTGAATGCCCCTTTCTCTGGGATAGATGGCTTCTTTGACTTA
ACAGTTATGGCCTTGTCTCTTCACTTTTCTCCTTTTTGTAGGATTCTTGTTGCTTAGAGAGGCTGGTTTG
CTTTCTTGTCTCAGTCAAACTATAAACTATAAAGGCAGGAACTGTGGAAGTCCTGTGGATTCTTTACTTT
TATTGTTTGGCAATGGGCGTGCTGTGAAGTAGTCAAAAACGTTTTGTTTAATGTTGGATAACAGTGGAGA
AGGCAGAGATGGGAGGATGTGAACAGCCTCTGAAAGGCAGTGTTGCTGGGCAGACTGCAGAGCCCAGTCA
GCATCCTGGGAGCACCTGAGAGCTGTTAGCTGCTACCAAGGCGGCCTAGACATGTTCCGTTGCCACAGCA
GACTAGCTCACTAGACTTTCCAAAATTTTTGACTACACTTAATTCTAAAGACATCTTTCGGGAATTTTCT
TCTTACTGTTTTGACTAGGTTAATACACTGTTTTGTTTTGTGAATGGATTTATAATTATTTGTTTCACTT
AATGTGTGTGTCTGATTCAATATATACATTCATGTACACATACATTCATGCACACATATGTACACATATG
TGTATGTGTGAGTATATTTGTGTATGTGATGTATGAGTGTGCATGTCTGACCACAGGTCAACATGGTGTG
TTTTCTCTGTCATGCTCCATCTTGTTTTTAGAGTCAGGGTCTCTCACTAAACCTGGAGCTCACTGATTAG
GTGAGCTCCAGGAATCTGCCTGCCTACACCACTGCACCCAGCTCTCCTTGGATGCTGGAGACCTGAATTC
AGGTCTTCATTCTTGCATAGTAAGCACTTTACTCACTGGGCCATTTCTCCATTCATCACTGTATATTTTA
ATATATCTTCTGGAGTCAGGGGCACATCTCTGAAGCCACACTTGAAGAACACTAGTGAGGATGAAGCAGG
GGGGTCACATACATGTGTGTCCAGCCTATGTTCTATGGAATGCATTCATACAAAGATGGCTTTGACTGTA
GCCCAACACAAAATCATAAAATACCTGGGTTGTTTTTTGTTGTTAAACCCTGATTGTGATCTTCTCTTTG
TAGATGACAGTGTTCTGTTGCAGTGTCAGTCAACAGGTTCCACGCATCTGCAAGGCTTTATGAGACCTGT
CTCAAAACAAGTTTATCTGGTTTGTTTTTTGGAAAGTAGGAATATAACTTTATGGTTTAAAGTGTACTGA
GACAATATGTAAAATCTAACAAAATACTAAAATGTCACTTTGCCTCATATGAGAGAAAAGGCTCATATTA
ATGGCTCATTTGTTTTTTGTTTTGTTGTTTTTTTCATTTTCAATTTGTAGAACAAGTTTTAGAAACTAAG
ATACCAAATGGAGTGAGTTCAAATCCAGATTTACCAGCACATCCTGACGCACACTTGGAAATTCACAGAA
AAAAGGCGGTTACACCTTTGGACATGAATCGCTTTCAGAGTAGAGCCTTCCGTGCTGTCATTTCACAGCA
GAGGGGGCAGGACCCCACCTTTCCACCAGTACGGCAGCCCCTTACTAAGGAGCCCTCTTTACACCTGGAC
ACACCATCAAAGTTTCTGTCCAAGGACAAGCTTTTCAAGCCATCCTTTGATGTGACGGTAAGGTTTGAAG
GAAATGTTACTAGTGCTAATGGTTTTATGGTTTATAAAGGTAGAGCCCCAAATTGTGCGTTACTACTAAA
GTTTGTAATAGAAAGTCATCCAGAGATGTTGATAAATGGGCGCTCAGAGCTGAGATAACAGCTGTGCCTA
TATAATATGAAGTTAGGACTCTGGGTTTGCTCATGAACACAGTTCTAGGCAGATGACTTAACCTCTGAAT
ATCACTCTGAAATGAAATAGTCTAATGTAATGGTTTTGTAAGCTTTAAAATAGCAAATGTGTTGCTACCG
TTTCTGGGTGACTTAGTGTGCCTTGGACTTGCTGAAAGCCCTAGTGTTGCCTTACCTTTTCAGTCACTCC
TCTTAAGGAGACAAGTGTTGTTTCTCTGTGTGATCTGAGCAGTGGGGCCAACACCTTATGAAATTTATTC
ATGAATCAAAGACTATTCAAATAGTCTTCTTTCAGATTCTCAAGATTCCTCCCCCCCCCCACCTTTTTTG
GGGGAAGGCTTTTGGCTTTCTAAGTAGCTGTTGAAGTAAGTCATTGCCTGAGATTTCTGTATCCTTGAGC
CCACTTACTTTTGATAGTTTGCTTATAGGATGTTAAGAAATGACATTTTAGCTGCACCTAGTAGTGTTTC
CTATAATCCCAGTAACTCGGAGGCCAAGGTGGAAGATCATAAATTCAAGACTTGTCTAGGTTACACAGTG
AGTTCAAGGCCTGCCTCAAACAAAAAGTACTTTTGTTTTTGTTTTGCCTAGAGTTTTTTGTTTGTTTGTT
TGTTTTGTTTTGTTTTTTTCTGAGATAGCTTTTCTCTGTGTAGCCCTGGCTGTCCTGGAACTTGCTCTGT
AGACCAGGCTGGCCTTAAACTCAGATCTGCCTGCCTCTGCCTCCCTAGTGCTGGTATTAAAGGCTTGTGT
CAGCACTGTCCAGCAATTCTTTGCTTTAAAAAAAAAAAAAAAGAGTTGTGGAGGTAGGTCTCTAATAGAA
TAGTGGCCTTGTGGACATGAGAAGACTATGGGTTTCATCCATAAGAAATCCAGGTGCATTTTAGACATTT
TTACTACTGTGTTTAGGGGCAGATTATTCCGAAAATAAATATTGATATCTAGTACCATTTTATTCTAGGC
TGATGTATATCCCTAATCTGAAAATCAAAATGGCTCCTTTAGTATTCTACAGTGGTCCTGCAGTTTTAAA
TCCCTTCCTCTGATTAGTGCTCTGCCCCATGAGCCTTCAGCAGAGGAAAGGCACTGTCTTAGTGCTATGA
CTTCTGGAGTCAGGATGACCATGCTTCATGCTGCCTTCTGTCTGACACGTGGGAACTCCCTTGAGCGTCA
CCATTCTTGTCCTGGGATCATGTTAAGTCACGGAATGAGTCCCAACAACTTGTACAGTTCTCAGACCCCT
TAAGTAGTAAACACAGAAAAGGGATGCACTGCTAAGTGAAACTGCAACCAACATTTGGGCAGCCACTGTC
CGAGCTCAGATAGGACTCATTCTGTGTGCCCTTCCTCATGCTCGGTATTTCTGTTTCTCCTCCCCACCAC
TTGTCAGAGTTGTGTTGTGCTTGGTTTTGGCACTTACAGAGGCAGTGTTAGGAAGTCTCTTGCATCTGTT
TTGTTGGATTTGTTTAGCCTCTATTGTGGAGATTTGCCCATGTTCTATATAACTGTAGTTAATCATTTAT
CATAAGATGGTATTTTTTCTATCTGAGGTAGTGTTTGCTATATATCCAGTATATTTATATGAAAACTGTA
ATTTCTGATTCCCAATATGTGCTTATTTACCCATCCTGTATATTAAGTGATGTTTGCAAGTTTCTTGTGT
TGATAATTCTTGTTTCGGTTTTTGGTTTTGGTTTTTGATTTCTCTGTGTAGCCCTCACACTGTAGACCAG
GCTGGTCTTGAACTCTCGGATTTACCTGTCTCTACCTCCCAAGTTCTGGGATTAAAGCCTGTTCCACCAT
GCCCAGCAGTGTTGGTAATTCTTCTTCTGAGATTTCACATGGTTTTGTTTTTTGTAAGTAGTCGCTTTGT
TAGTTGGCACACTGATGTGTGACTTGTATTAATTAGAATATAAAATGCAACTTTTGATTTTATAAAAGAT
CCTGTCTTGTTTAATGCTTTCTGGCTTGAGGGCTGTCTGCTGTGGTTTTAGGATTTCAGTGTGCTGTATT
TATCTCAGTTCTGTCATGTATATAGTAACTTACACTTGCTTCATATACCTTTATATTATGTTTGCTTGAT
GTTCTTTTTTCCTTGTTTCCTTTTTGTATGTAGAATAGAACCCCTGCTCCACTTTTGAGACAAGGTCTCC
TATATAATGCAGACTAACCTTGAATTCTGAGATCTACCTGCCTCTGCCTGTTGATGACTGGAGTTAAAGG
CATGGACCTCCATGCCTAGCTTTTTCTCTTTTCTTTCCTCTCCTCTCCTCTCCTTCCTTTTTTTTTTTTT
TTTTTTTTTTTTTTTTTTTTTAAGATTTTAAAACAGTTTTACTGTATAGATAGCCCTGATTGGCTTGGTA
CATAGAGCCAGGTATTCATCACTGTTGTGTATGATGCTGGAGAATATGCCTAGGGCTTTGTGCATGTAGT
ACTCCCAGCCGAGCTGTATCCCAGTACAAACCATAAACATGTGTTGATCATTTGCCATTGTGGTTACTAG
CTAGTAAATTCCCTACTCTTTGCTTAGTTTCAGCAAAGAGTAATTGAGGCTTTTCCCTTCTCTCCTTTCT
AGCCTCTCCGAATCTTGGGAGAGTTAGCATTAAAGGTGTATGGGATTGTCAAAGCCTAGAGTTGACATGA
CAGGTAGTGCATCCTCTCCATATTAACTGTCCACTTCTCCTGCTGGCATATTTTACTTAGTTTGGATATT
ATAAAATTTATGCAACTGCATTCCTGAACATATTCATTCATATTATTTCTGATCTTTCCTTCTTGAGGTG
TGTCACTTTTTGTTAATCCACATCAACAGTAGAAGGATCTGTTCTCAAGAACTTTGTTTCTCAAGAACAC
TTCTCTCTTTGTGATCATGCATTCCTTGGTATCATGAGGCTGGATGAAAGAAAAGAATGGCTTCCTCCAT
TGTAGTCTGCAGAAACCCCAGATGAGAGGCATCTATCTATCTGTCGTAGAACTTTCAGCACCCATTGTCA
TGAAGGGATATTAGAACTGTCCTCTAAGGTGCCCAGAACACTTACGCCTGTCTCTAAAGGTGTCAGATGC
TTGCCCACTTTCAGCGTTAAGTGCAGTCCTGGTTAGCTCTGAGCTCTGTGGACTGCTTGGGAAGTAGCCA
GACCCTGCATGGAGCTCTCTGCGTCCAGTTCTTAGCCTCCCCTCCCTTTGAGCCTATATTTTATATCTTG
TACATTTTGTTGGTTCTTGCCTCAGTCTGACTTTTTACCTAATCTTAAAATTAATTTCTTATACCCATCC
ATTCTTTCTGGTTTCCTTCTGTGTTCACTTTAGTTTGGGGTATTTTATGTAATTGCAATTACAATTCTTA
AGTGTGGTATGTATGCACTGCTGAAAAGGTAAGTATGCCTAATGGTTTTGTTTATCTGTTTTAAAATCTA
TTTTTTATGTGCATGGTTCTTTTGCCTACACATATGTTTATGCATCACCTGCGTGCCTGGTGAACATGAA
AACCAGAGGGTGTCAGATCCCATGGAACTGGTGATACATATAGTTGTGAGCCACCACGTGAGAGCAGGGG
ATTGATTCCCTGGGTCCTCTAGAAGAGCTCTCGCAGTACTCTTAACTGCTCAGCCAACTCTCCAGCTCCT
TACTTAATGCTTGGTGAAGAATATGAAATTCTTTTAACTCTAGTATAAAAATAAAAATTAAAGTTCATGC
TTTTTATTTCAGAGAAAAAGAGGTCAAAGATGTAATCTCTTTTTTCCTTTCTTCACTACTTTGTCTCTTT
CCTCTGTGTCCCCCTCTTCCTCTCCTCATTTTTTCCTTTTTCCTGTTCTTAAACACAGGATGCACTTGCA
GCCCTGGAAACCCCAAATGCTGCCAGCCAGAAGCGGAAACTAAGCTCACCACTCTCTGAAGTCATCGTCA
GAAACCTGACAGTTGCTTTGGCAAATAGTTCCCGAAACACTGACAGTCATTCTGCCAGCCCTCAGCTGAA
GGGTGCCCATTTGGAGGAGGTAGACATGGTTTTCTTATTAAATGGAAGGTGTCCCTAATGCATGTCAAAG
AACTGTGGCTGGTCTGTTTCTTTATTCTCCATGTGCAGGAAGAGACTCGGAAACCACTTGACAGCGTTGT
GGTGTGTGTGAGTAAGAAGCTCAGTAAGAAGCAGAGTGAGCTCAATGGGGTCGCAGCATCACTGGGAGCA
GAGTACAGGTAGGGACAGCTCTGTGTTGGCGGCGCCAGCCACAGCTCAGACAGCACAAAAAGCCTGTATT
CATTTATAAGGAAGACTTGGCTTTGCCTGCTTGTGTGCATTATCTAATAAAACCTCTCCGATTAATTAAA
ACTGTCCTCTTAGGTACCCAGAACACTTAAGCCTGTCTATAAAGGTGTCAGATGCTTGCCCACTTTTAGC
CTCCAGTGCAGTCCTGGTTATGGACTGGTCATGGGTTTATAGTCAGCACATCTGGGACTGACTGCATTTA
TTATCAATCTTAGTGATTTCCTGTGAGTAGTACTGACTGACCACTGTAGCAAACCTTCCTAGATTCCCCT
GACTTCCACACTCCATTAAAGACATTTTAGTTTGCTCTAAATAAAAATTTATTTGTGTGTGTGTGTGTGT
ATGTTGGCATGGTGGTACACTCCATTAATTCCAGCGCTTGGGAGGCAGAGGCAGGAGGATCTCTGAGGGT
CTACATAGTTCCAGGATAGGCAGGGCTACAGAGTGAGAGACCCTGTCTCTAATGTTTTCTTAAGAGACGT
AGTCTCATGCTGTGGTAGATTTCTTGCCTAGTGTACTCAGAGGCCTGAGCTCAGTTCTCAACATAGATAG
TTTTGTGATAGGGACTAACTTGATAGAGTGAATAAACAGGGTGGTGGCCAGTGACTGGACAGTGGATTTG
AATGCCTTAAGTTTTATTAACTTGAAAATACTTTTTATTGTAATTAGAAAGTGATATACATTATAAGACA
AAATGGAAGGATTCTTAAAAATATAAAGAAGATAGTAGTATTTTAAAAGTATTCTTCTGGCATCTACACT
GTATAAATGCTGTGTAAATGGAGTCGCTAGCATTTGTCAGGATACAAAATCTGCCATTTACTACTCTTGC
TTATTCTCTAGTTCTTTGAGTTCTGTGTGTCAGAACATTCTGTTCAGAGTAGACTGCTGTTTCCCTTAGT
TATTCCTCTTGCATATTTGGTTGTTTCAGGTGGAGTTTCGATGAGACTGTTACTCATTTCATTTACCAGG
GTCGGGCAAATGACAGTAATCGAGAATATAAATCTGCAAAGGAAAGAGGGGTGCACATCGTGTCCGAGCA
TTGGCTATTAGAGGTAAGCAATGCTTTTCCCTCTCTTCCTTGTTGGTTGAAAATACACAAATATTTCAGA
GCACAAACCAGTTTCATTGAATGCAAATAAATTTGCTAATCTTGGCTAACTGTGTGGTGGCATGTCGTGC
CAATACCTGCCCATTTCATGGCTGGGACATGAAAGAGGTCGTGAAAGGGTTGGGATTGGACTGCTGTCCA
GGGCACACCCCGAGGGCCTGTTGGATGCCATGGGAACTGTGCTTGTAACATAAGGCCTTGGGACACTCAA
AATCTGAGCTATATGTAGGTCATGCTTGGGGCATTGTATCGTTTTAGCATTAGAACTTAAATTGTTTTAA
GATTAATTGGTTGATTGGTTGATTGATTATTTTTGTATTTTTATGCACATGAGTGCTCTATCTACTTGTA
CACTCGAGTGCCTAGAAGAGGGCATCAGACCCCATTATAGATGGTTGTGAGCCACCACGCGTGATAGATG
GGGAATTGAACTCAGGACCTCTGGAAGAACGTACATCTCTCCAGCCCTAGAACTTAAAATTTTTATTGCT
TGAATATTTACCAAGTTTTAAATGAATACTTATATCATGATTCCTTTCCCTCATTGACTATTAGATGAAG
AGTAGCTAAGGGGCTGGAGAGATGGCTCAGTGGTTAAGAGCACTGACTGCTCTTCCAAAGGTCCTGAGTT
CAATTCCCAGCAACCACATGGTGGCTCAAAACCATCTGTAATGAGATCTGATGCCCTCTTCTGGTGTGTC
TGAAGACAGCTACAGTGTACTTACATATAATAAATAAGTCTGAGAGGGAGAGGGAGGGAGAGATTGAGAA
AGAGAGAAGCTGAAAGAAAGGGTGTGGAAGGTAAAGGCTCTCACCACCAAGCTTGACTATTTGAATTTAT
TTCCTGGGACCCATACAGTGGGAGGAGATAACCAACTCCTACAAGGTCTGCCCCCCATATAAATAGATAG
AAATTAATTTAAATCATATAAAAACAAAAGATGTTTTTGAGGGTAGTTGCTAGCATCCTCATACACTATA
GCCATGCTTTTCTTGGTGCTCTCTGGAATGGCTGATACAGAGGCTAAATGCAGAGGCAGGATTAGAGTTT
ATACTTTAATAGACAGTAGATTGTAAAAGTGGGGATAACTTGTTCCACTTTGTTTAATTTACTGTGTGTG
CATAGGTGTGCTCAGAGGGCCTTTTACTCTTTTTACAGTTTTAATATGGTTTCAGTGATTAAACTCATGT
CACCAACTTAATAGGACAAGGACATTTACTGGCTAAGTCATTTCCTGGGTCCCAATTGGTTATTTACATG
CTAGAGTTTTCCACTGTAAATTAATTTCCTTCCTTCCTTCCTTCCTTCCTTCTTCCTTCCTTCCTTCCTT
CCTTCCTTTCTTTCTTTCTTTCTTTCTTTCAAAGATTTATTTATTTTTTATATGTAAATACACTGTAGCT
GTCTTCAGACACTCCAGAAGAGGGCATCAGATTTCGTTACGGATGGTTGTGAGCCACCATGTGGTTGCTG
GGATTTGAACTCGGGACCTTTGGAAGAGCAGTCGGCACTCTTAACCACTGAGCCATCTCACCAGCCCCTG
TAAACTTCTTTCTAAAGTGTACACTTGTGTTTCAGTGTGCTCAAGAGTATAAGCATCTTCCTGAATCTCT
TTATCCACACACTTACAATCCCAAAATGAGTTTGGACATCAACACAGTGCAAGATGGGCGGCTCTGTAAC
AGCCGGGCACCCTTGGCAGTTTCTGCATCTAAGGATGATGGGGTAGGTGGTGTGGACAGTGAGCCCCTCC
CAAGCTGCATGTAGATGTCTGTGAAACTTTATGCAGTGAACTTCGTTTGAGAAGTTGTCATTTTTCTCTT
TGAATAGCCAGATCATCTGTCTGTAGAAGGAAATGAGACAAACACTATGGGCACAAATGACAAAGAGTCA
CCTCTGTTGAATGGGAGTGGAAGAGATGACTGTAAAGGAGGTAAGACACTTGTCACTTTTTACCCCTTAC
TCTCACGCCATGGCCTCCCAAGACAGGGTTTCTCTGAGTGGTTCTGGAACTGACTCTGTAGACCAGGCTG
GCCTCAAACCCAGAGATCTGCCTTCTTCTTTCCAAGTGCTGGGATTAAAGGCCTGCGCCACCACACTCAA
GTGCACTTTTTACTTTTGACAACGTTTGTTTTGACTTACAGTCCCAGCTGTATTTTACCACTAATAGAAG
AACTGGGATGGCCTTCCAGCTTTGCAGCCAAACACTGACTTCTCTCCACAAAACCCTAGAGAGGTGTCTT
CTTCCAGTCTGAGTCAGCTGCTCATTCTCCTGCAGCTTCGCATCAATGAGGAGCTGTGCCCACTGCAGGA
GCACTTGGAATTTCTCTTTGCAGTCACAGCTTGACTGTTCAGCAGAAGAGCCTTTTGACCTAACTTGACT
TTCATGCATGCCTTTGTCACTTGCCTTAGTCACTGCTAGCTTTACTTTAGAGCTAGAGAGAGGAGTGTGA
CTCTTCTCTTCACTTGAACACTAAGCCACTGTAGGATGTTAATTGAGCTAATTTTAGAACTGTTGTTTCT
TAGGGATTGCGGAAGTCCTTGGAAGGAAAGAGAGGAGGAACAACCAGTCAGCATATACAGCTTTTATCTG
TAGCTTTTGTGTAGATATGGTTCACACAGAATCATTTGGAAGAGTTGGACAATTGTTAATATATGGTTGC
TACAGCTCTCCATTTGCAAAAAAAAAAAAAGAAAGAAAAAAAAGCAATGTTTTAGTGTAGCTGGAGCTGA
GAAATATATAGTAAAGGGTACATTTTTAGCTCAAACAGCCTTAGAAAATGAAAGCCCAAGCTTGATTGGT
TTGGACTCTGATGAAGACCTGGCTACATCCTATCATGTCACTATCAGAAAGGGAAAGAGTGCAAGTGGGT
GTCACCACATGAAAGACTGCAAGCAAGTTAGACCAGGCTCACTGTTGTCATAGCAACCCACAACAAAACT
AGCCTGAGTTCCCCCTGAAAACTGCATTACTTCTGGGGTGGTGTTGCTAATGACCCAACCCCCTTAGACT
AGATCTCACTCTTAAGGAGTCTCCTTACTATACCACACCACACACAGGAGGGTGTGCACTCAAGCCAGAG
AAGAGTGTCTGGGAAACAGGGGCTGCTCCAGTCCTGGGTTTTATTTGCTGGTGTAGTGTGTGGCTTCTGT
CCTGCTTTATTTTGCTGTTTCCTGTTCAGTGTCACTAGCAGTTATTTTGAATTTCTTGCTATTACAGATA
GTGCTGTGCTGATTACCTTACTTAGATGTGTTACTCTCTGACTTCTCCTTATTCAGCCTGCCTACTAGAA
CTTGCTTTCTGACTTAACTGTTCCATTCTGTTTATTTCAAAAATGCCTACATGGTATCTACTTCAGTGTC
TATAATGGGAAGGAAAGACAAAGACGTTGTGGTGTGTTCTTTCAAGGATTAGTGAAAGCTCGGCTAGTGC
TTTACAGCAGCAATCCCAGGCTTGCTGCTGAGGCAGGAGGGCATTCAAGGCCAGCATGAGCTATGTGGCA
AGATCCAAAGCAAATGGACAGACAGCTATACATACAAATGACTGTGAATCTCTGTAGTATGCATCCATGA
GAAAGGGTCAGACACGAGCAGGGAGCTCCTCAAAGGAGCAAGTTTTCCTCCAAGGGAGTCTTCATCGCTT
CAGAAACAGTCCTCATGGCCGCCTTTGTGTTTTGTGACATTTTAGCCTTGTATTCCTGTATAAATGTTCA
GTAAATGGCAATTCTTTGTTCAGCTAGATCTAGACAAAATAGTGTAAGATATGATTGACAAGAGAAGTTA
GTAACTGTGGTAATGCTTGCCTGTAATCCCAGAACCCTAGAGACTGAGGCAGGGATTACAATGGGAGGTC
AGCCTGGCCACACAATCAGATCTATCTTCACACCAGCAAGGTGGAACGGGGGGGCACATCTTTCTTGATT
GATATCGATTACTCTGTTTCTAGCTCTGACCCAGGCCTTGGAGATGAGAGAGAACTTCCAGAAGCAGCTG
CAAGAGATCATGTCAGCCACCTGCATTGTGAAGACGCCTGCCCAGAAGACTTGCATGTCAAGAAGCAGCT
GTAACAGCGCCTCTTCAACTCCTGACAGCGCTCGCTCTGTGCGGAGCGGACGAAGCCGAGTCCTGGAGGC
ACTCAGGTTTGTGCGCTGTGTTGTTTGCATAAAAGCCCCTGGTAACTTAGGTTGGTGGGTTAGGCTCTTT
TTGTTGTTTTTGTTTTTCGAGATAGGGTTACTTTGTGTAGTCCTGGCTGTCCTGGAATTCTGGAATTTGC
TCTGCATACCAGGCTGGCCTTGAACTCAAAGATTCATCTGCTTCTGCCTCCCAAATACTAGCAATAAAAG
GTTTGTGCCACCACACAGGACTTTAATTTTCTTTTCTTTTCTTTCTTTCTTTTTTTTATTTTAAATAAAG
TGGAAATTAGAATATTGAGACCAGTGAAGGGGTTTGTGTTGGCGCTTTGTTTAACTCAAACAGTATGCAT
TATATCCCACTGTCATCTGGGGTTTTGTGTATTGCTGCTGAGTTTTTTGTCTTGTTGCTCAAGCAGTTGT
GAGTTCTTACTTTGAAGTATGTGATTTTAGGTGCACTTTTCCACTTGACGTAAAATCCGGGAGCAGTCAG
TCCTCTCTGTCACTTAGTGTTAGCACTAGACAGTGAGGACACAGAGTACTTCAACTTTAAACAGCTTTTT
TATTATCTTGTGTTTGAGTGTGATATGTGGGCGTGAAGACATGCATGCCATAGCACCCATGTCGAGGTCA
GAGGACAGCCTGTGGATGTCACTCCACCCTTTCCTCACTTTTTATTTCTTACTTGAAGTGTGTGTACGTT
TTTCCTCTGTGTTCTAGGAAAGGTGCCACAGTTCTTAATGCAGTATAACTTAAGCTCATTGAAATTACTG
TATTTTTCTTTGTCTTTATAGTTATAAGACAAGGTCTTATCCCAGGCTGGCCTGCAATACTGTGGAACTA
GTGTTAGCCCTAAACTCCCAATCCTCCTGCTTCAGTCTCCTGACTGCTGGAATTACATGTGTACCGCCAC
TCACCCTCTCTCTGTATTGCTGTGTAGCCAAGGTGGGCTTTAAATAAACTTGGTGTTTTTCTACCTCAGT
TTACCAAGTGTTGGGTTACTGGGCCCACCAATAGCATTTTTTTACCTCTCTCTGTATTGCTGTGTAGCCA
AGGTGGGCTTTAAATAAACTTGGTGTTTTTCTACCTCAGTTTACCAAGTGTTGGGTTACTGGGCCCACCA
ATAGCATTTTTCTACTAGAAGAAAAAATCTCTGAGGTGTAGGGTGAGAGATTTGAGTCCATGTTGACTTC
ATTTATATCGTACTCTGTGTTTTCCTTGGGGTAATTTTGAATTCTGAGAAAAGTAGCTTTTCCCAACAGT
GCAGGTCATGTTTTAGATATAGAACCTTACTCTGTGCCAGGTCGTTTGGGACTAAAAGGCTTTCCTGGTT
CATTGGCTTGTTTGTTTTTAAGACAAGGTCTCACTATGTAGCTTGAGCTGGTCTGGAGCTCAGAGATCAC
CTGCCTGACTCTGCCTCTGCCTCTGCCTCCATCCACTTTGGGATGTAGTCCTTACTGTCCTGAAACTCAA
TATGTGGACCAGACTGGCCTCAAATTTACAGAGACCCGCCTCCTACTGGCTACCGAGTCACTTACTAACT
GCATGTAGCTTGGTGATGGCGCAGGCCTGTAATCAAGGCCCCAGGGAGATAGCCAGGTGGTAAAGGTGCT
TCACGCCAAGCGTGATGACTTGAATTCCGTCCCTGGAATGGTGGAAGAAGAGAGCTAACTTCTGTAAGCT
GTCCTCTGGCCTCCACATAAGGTCACTGTGTCACATATGTGTATACATACACACAAAGTAAATAAATAAA
TGTAATGAAAACATTTTTAAGGGACTAGAGAGATAGCAGTTAATGAGTGCTTGCTACTCTTGTAGAGGCC
ACAACTAACTGCTTGTGACTACAGGTCGAGGGGATCTAACAACTTCTTTTGGCCTCCCTGGGCACCCACA
TCTGTACATATAAACAAAATACTTGAAAATATTTTAATGAATTCATAATTCTGTAATTCAGTAAATGTCT
ACCATAGACTGGAAATTTTGGTCACCAAGTGTACTGGCTAGTTTTGTGTCAACTTGACACAGCTGGAGTT
ATCACAGAGAAAGGAACTTCAGTTGAGGAAATGCCTCCATGAAATCCAACTGTAAGGCATTTTCTCAATT
AGTGATCAAGGGGGAAAGGCCCCTTGTGGGTGGGACCATCTCTGGGCTGGTAGTCTTGGTTCTATAAGAG
AGCAGGCTGAGCAAGCCAGGGGAAGCAAGCCAGTAAAGAACATCCCTCCATGGCCTCTGCATCAGCTCCT
GCTTCCTGACCTGCTTGAGTTCCAGTCTTGACTTTCTTTGGTGATGAACAGCAGTGTGGAAGTGTAAGCC
GAATAAACCCTTTCCTCCCCAACTTGCTTCTTGGTCATGATGTTTGTGCAGGAATAGAAACCCTGACTAA
GACACCAAGGGTTGAAAGACCCATTAGTTATATCTTCAGCAATGTCATGTTAAAAATACACTTTAAAGCT
GGGTATAGTGGCACCCACGAGGCAGAAGCAATGGATCTGTGAGTTCAAGACCAACCTGGTCTTCAATGTG
AATTCCAGGACAGCCAGACCTACATAGTGAGACTCAATGAGATATAATTTAATTACTAAGACAGGTACGG
TGGTGCATACCTACAGTCCTAGCACATGGAAGCTGCTGAGGGGAGCTCATGGTCGTCCTTCACTACAGAG
AAGCGTCTTTAAAAGAAAATGTAAATGCAATTATTAACCCACTAGCTACAGTTCTCTAAACATCACCCTG
GTTCCCTAATTTTTTAGAATTTAAAAAATACCCGGGCTTGATGGCGCACGCCTTTAATCCCAGCACTAGG
GAGGCAGAGGCAGGCAGATTTCTGAGTTCGAGGCCAGCCTGGTCTACAAAGTGAGTTTCAGGACAGCCAG
GGCTACACAGAGAAACCCTGTCTCAAAAAACAAAACAAAAAAAAATTAAAAAATATATAATTATTTTTGA
AATACAGTTATAGACATTCACTTAAATTGTCTAGTAGAAATATTTTAGAAGTAATTAACTGGGTGTATTG
GTGCATGCTATGCTGTGTAGGTTTTTGTAAACTTGGGTCATTTAAGAAGAGAAACCTCATCTGAGAAAAT
GTCTTTATCACTCTGGCGTGTAGGTAAATCTGTAGAATATCTTGGCTAATGAGTGATTTAGGAGGGTCCG
GCCCACTGTGAGCCCCTGGGACTTCCTTTGAGCAAGCCATGCAAGTCAATAAGCAGTTCTCTTCTACAGT
CTCTTCAGTTCCTGCCCGGACTTCTCTCAGTGATGGGCTACAGCTGTAGGCTGTAATAAATCTCACCAGG
CTGCTTTTGATCATATCTTATCACAGCAGGGGAAGCAAACCAGGATGCATGCCTATAATCTTAGCACCTG
GGAGGCAGAAGCAGGAGAAGCACCTCATTTAAAGCCATACTCTCAATAATGAGACCATATGTCCAAAAAA
AAAAAGAAAAGGAGAAGAAAGGATGTCCTCTTGATTTATTCAACAAATACTGTGTGTCTTTGCTGCTTAC
TGGTTTTCCGCACTTGGGTTCAGTGTGGCTGCTATTTAACACTGTCCCTTTTGTTCCTTAGGCAGTCTCG
GCAGGCAGTGCCTGATGTGAACACGGAGCCGTCCCAGAACGAGCAGATCATTTGGGACGACCCGACAGCA
AGGGAAGAGAGAGCAAGGCTTGCCAGCAACTTACAGTGGCCCAGCGACCCCACACAGCATTCTGAGCTTC
AGGTTGAGATAAAAATGCCGGATGACTCTCCCTCTCGAAAGCCTGTGTACCATTCAGAAATTGCTGAGCA
GGGTAATAAGACAGACATTGACTTGTGCTTAAGAATGCTGACCTCTTAAAGACACCATGGTTCTCTGTTA
CATATGCGAATCAAAATGAATTTTCTAGTACTGTTTCAGTCAAATGAGAAATGTTTTTTGATTTTTAAAT
ATAGAAATTTAGCTATAAGTTTCTGAAGAAGAAATTGTCATTTTTGAAGCTTCTAGCCTCCATACAATCT
AATCTGGGGCCAGAATATTATCAGCCTCTGAGCCTTACTGCTGACTTAAGTTCACCCTTTCTAATTCTTC
TGAGCTCTAGCTGGATGGCTCAGCTTAGCTGGTCAGCTGTAAACTCCTCTCCAGAGTGACTGATTCAGTC
TGGCTTCTCTCAGCTTCTCCTGAACTGCTCTGCTTGGCCTCAGACTGTCTTTGGCAACATGTTCTAATCT
TATTGCTTCTTCTCGTTCTCTGGCTTGTTCTGTTTTCACCTGTGTTTAGCTGTTCTCTCTGCAACCTGTC
CTTCTATAATAATGTCTGGTAAAACGGCCTTTCTTCTCACTGTGCTGCTTCCTATAGCTTCCCTTTCCTC
TCCTTGTTATTGGGCCTGTCCTAGTCTGTCAGATCTTTCTCTTGATTCAGCACTTTGTCTGCCACTCAGT
TAGACATGGCTTACAAACATGGGTGCTTTTTTTCTACAACTAACTTTACCTTCATTGCTGGGAATTAAGA
GCAAGTCTGTATTCCAGCCAGAGGGATTAAAGGTGTGTGCTGAAACTCAACCACACCACAAGTAAAAACA
GGTTTTCTCAGTAAAGATCACAATCTTGGGGTTCACCGTGTGATCAAATATCCTGAAATATTTAGGATCA
GCCTTTTTTTTTTTCCCTTTCTACCCTCTTCTGTTTGTGAGGTAATGTCTGATGTTGTTTGGCTTATGAG
CTTTGCCTGAGTTCCTTTTTTTTTCTCCCTTCACCTCGGTTAAGGTTATCTTCCTGTGCTGGATATACAG
ATCTGATTTTTTTCTCCAAGAATTGCTTTCATTTTAAAAAGTTGGGTATGTTATACTTGTGAAAAGATGT
TTTAGAAAGGGATTCACAAAACTCAATTAATAGCTGCCCTTTCAGAGCTATTTAGTCAGATGTACCTATA
AGCTGGGGAAACCAACCACTCTTTCCTCTATGTGTAGTGCTTGCTTTTGAGCCATGCAGGGTTGTACCCA
TGCAGGGCTCAAGCCTGAGAGTCTCCTGTCTCAGCCTCCCAAGTTACTGGGGTTACAAACAGATGCCACC
ATGTCTGAGCCTTTGTATACTTTTGATTGCAGAATTCATGGCTCATTTTATTTTGGCCAAGACAGGTGAG
AACATTTCCAGAGTTTGTTCTAAGTTCCATGTGTACACTCTTACACAGTATTTCTTTCTCCTTACCCTTA
GCCTCGTGTGTTACTCAAGCCCCAGGGCACCCAGGCTCTGAAGAACCAGAGCCTCCAGTAGCAGAAAGGC
CTTTGATCCCCGAGCCGCAAGCCCCCGCTGTAGCTTCCCCCCTGGCTAAGCCGCCTGTGGCCCCACAACC
TGCAGACAAGGTTTGTTAACCTTCGGTATGGTGATGGCGGGCGGTTTTGTGTATTTTATGCTGGAGTAGG
AAGTGTTGGCTGCAGCAGGCTCATCACTACTGAAAAGTGCATCTCTTTGAATTACAATTTGTATACTTGG
TCCCTTAGAAGACTAGATAAAATGTCACTATAAAATAGCTATATCTGATAATAGTTTTAATGTTAATATT
TTTCTAAAATAAAACTATTGTGTTTTAGATTGAAACACAGGAGGAGACGCACAGAAAAGTTAAAAAACAG
TATGTGTTCCAGATGTCGTCTCTGAATTCTCAAGAGCGGATTGATTACTGTCGCCTGATTAAAGACTTAG
GTGCCATTATTTTACTTTTTTTTTTTTTCAATTTTCTTCCAATATTTTACTTTAAAACATTGTTATTACA
ACTTATTTAGAATACAAATGCAAACATATTTCTATCATGTGTAAAGGGGAGGGCAGTTGAGAAAAGAACA
TGGTTAAGTTTTGGATTTTGTTGGTTTTTTTTTTGGTTTTGTTTTTTTTTTTTGAGACAGGGTTTCTCTG
TGTAGCCCTGGCTGTCCTGGAACTCAGTCTGTAGACCAGGCTGGCCTCAAACTCAGAAATCCACCTGCCT
CTGCCTCCCAAGTGCTGGGATTAAAGACATGTGTCACCACTGTCTGGCTATGGGCAAGTTATTTAAGCCT
ACACATTGAGTCTGTTCTGCCTCACAGACCCAGGTACACAGATAGGCAAAATACACATACATATAAATAA
TATATATATATATATTTTTTTTAAGTCAAGGGTGGGACTGGGGGTTGTATGGCTCAGTGTGAGTTTTTTT
GGGGGGGGGCATATATGAAGCCCTGAATTTCAACTGTATACACGGGTCAGCATTGGAAGTTTTATTTTTT
ATAAAACCCCAATCTACATTTCAAATCACTAGATCTTGGAACGCTGAAGGCAGGGATGGAGGGCGATAAA
TTAAAGTGAATGTTACTTTTACATCTCTAGGTTGATCATTTTATATAAATAATTTTTATGCACAGTTAAC
ATTTTTCTAGAAAGAAAGTTAAGAACTAGTAGATGCCGTTTCTCTCCATACAAGGTGGGTCGGTGATCGA
GAAGCAGTGCTCTGACCCCAGCTGCACACACATGGTTGTGGGCTACCCACTGCGCAATGAGAAGTACTTA
GCTTCTATGGCAGCTGGTAAATGGGTGCTTCATCGCTCCTACCTAGACGCTTGCAAGACAGCTGGGCGCT
TTGTGCAGGTGTGTATTCATATTCCTGTGAAGGTTTGTTCTCTTGTTAATGACTTAGGAGATCCTTGGTC
ATCCCCTCACCGTGTATTTAGTTTAAATGACCACACGTGGCCAGTCAGATTACAGCTGTTCTGTGAGGAT
CTTTGAAACAGAACAAAGTCTGCAAGGTCGGGCTGCTTTATTCACATCCCTTTTTATTAAAATATTTTGG
TTGGCATACAAAAAAATGAGTTTTTATTGTGGCCTGCTCACTTTTTGTTACCTAATTTATCGGTGCTTTT
TTATTGAAATGATTTTCTCTTAATGAGCAGATTCAAGATAGCCCATTATAAGGCTTTTTCTTTGCTGTTA
GCTTATCCATATTAAGCAGTTGTAACAAGCTGGGCATCACGGTAATATCTACTGTGTCGTTTCCGAAGCT
TCCAATCTGAGCTCCTTGGGACTTGTCTTAGTTCATACCTCCTCAGGCTGTGCTCTCATGAAGCCAAATA
CTAGGGTATTTTACTAAGTTAGGTATGCTACTAAGTTGCCAAAATACAGCTGTCTGGCCAAAAGGTTTGT
TTTGGGGAAATTAGTTTCTGGTTTTAATTAAAGCCAGCTGTACTGGTTCTTCACATAAAACTGAATAAAT
GGAATGAGTTCCCAAAGTAGTGTTCTCTATCATTCGTGTCTCTGTAACGATTGAACTTTCAGCTTTGATC
TTGATAGACTGCAAGCTGCTGGGGGCCAGATACCCATGCTTAGTTAGCACAGTGCTGTGAAAGGAACCCA
CAAGTTCACTGGCCTGGTTTTCTAGGAAGAAGACTACGAGTGGGGAAGTAGCTCCATACTTGATGCTCTC
CCTGACGTCACTGAGCACCAGCAGAAGCTCGCACTGGCGGCAATGAGATGGAGGAAAAGAATCCAGCAAA
GTCAAGAATCCGGCATTGTTGAGGTACTATTGTGGGATTCAGTATTGTGACTTGTTTTAAAGAGCATATG
CCTGACCAAGTAGTTCTGATTGTCTTTTATTCCATAATTGATATATTTAGTTGCACATTCTGTGTTGAAC
TAAAATTTCTGTTGTGCAGGGAGGCAGAGGCAGGAGAATGTTTGAATTCCAGGATATCTGAGGCTATGTA
GAAATCCTGCCTGAGACAACAAAGTATTGTGTTACTGTAAGATAGGGTCTTCTAAAGATAACTTGTCTTT
TCAGGGTAGAGCATCAGGAGTCCTAAGCCAGGTCTGCAAATTCTCTCTAAGGAGTCTATATACTTTTGAG
TAAGGAAATTATGTCCTTTCTTTCCTAATTGCTGATTGCTAGGGTACTCCTGTAATCCTAGCACTTGGGA
GGCAGGAGAAGAGCAAGAGGTCAGAGCTAGCCTATGAAACTTTGTCCTTTTTCTTTTCTTTCTTTCTTTT
TTTTAAACTAATAATGGGTTGAAGAGGCCGCCCAGGGGTAAAGGCACCTGCGCTGCTCTTCCAGAGAACT
CAGATTTAATTCCTATCATCAACATCTGTAAATCTACTTCCAGAGGATCCAACACCCTCTTCTGACTTCT
GTAGGCCCTGCATGCAAGTGATACTTAGTATATGTAAATATATGCAAAAAACCCACTGAGTTTTGTGTGT
GTGTGTGTGTATAGATAGATAGATGAATGACAAGAAATTACTTGACACTAAACAATTAAATTCATGCATT
ATCTCTGCATTTTGAATGTTTAGTATGTTAAAATTACATGTACTTATTTTTAAATAGGGAGCATTTAGTG
GCTGGAAAGCTATTTTACGTGTTGATCGGCCCCGAGAAGCTGGGTTCAAACGCCTTCTTCAGGCAGGAGG
AGCAAAGGTTTGTCCTGAATTAATTTACTGCTATCCTAGGGGCAGGTTTGCACCAACTTGTCCAGCAAGG
TTTCTGTGATGTCTCCCTGCCCTCTGCCTTTGAGGGGAGGAGCATCTGCTATGGAGCATGAGGGGATTAA
CCCTTCCTGTTTTTCATTAGGTGTTATCTGGTCATCCAGAACCTTTACTTAAAGATGCTACGCATCTCTT
TTGTGACTTCAATAAACTGAAACCTGATGACTGCAGAGTTTTCATCGCAGAGGCTACTGCCCAGAACATG
GTCTGCTTGAAAACAGAATATATCGCTGATTATCTCATGCTGGTTTGTCACATTCTACTCTTCCGTTTAT
CATTATCATTTTTATTACTTGTGTGTATATGTGGGGTGGGGAGTTGGCACATGTGTGCTCGTCAGAGGAT
AGCTTAATGGAGTTGGTTCTCTCCTACCATGTAGGTTCAAACTTAGGTTGCCAGGCTGGTAGCAAGCGCC
TTTACTTGCTGAGTCATTTTGCTGGCCCCATACTTCCTTATTTCTAGAGATATGTGTATGTTTGTAGATG
AACATCCACACATTTACTCTTGGCTTGCTGCTAATAGAATTGACAGTCTGCCATTCTCTAGTCTTTCAGT
TTAGTTCCATTTCTATATTGGATGCCTGTAAACTTTTTAGCATGAAATTTAAGTATGACAGGTTTATGAA
AATCTGGGTAGCTATAGATGTTTTACAAGTTCTTTAAAATATTGGGCTATAAAGAAAGCAGGGGGCTAGA
GAGTTGGCTCAGAGACAAGAGAACTGGCTGCCATTGAAATCAGACTCAGTTCCTACCTAACAGTAACAGA
CCTACCGTGTGTACCTGTGTTCGTGTGTACCTGTGTTCGTGTGTGTGCTCGTGTGTGTGTGTGTGTGTGT
GTGTGTGTGTGTGTGTGTGTTCGTGTATAGAGGTGTCTGTGGAGTTCAAAAAAAAAAAAAAAAAAAAGAG
TTCTATTCCCTGGAGCTGGAGTTATAGGAGGCTGTATCCTGTCCAGTATGGGTGTGAGAATCAAACTTAG
ATCTCTTAAGTGCTCTTAACTGCTAAGCCATCTCTCCGGCCCTAAATGAATGGATTTTTAAACTGTATAC
TTGCTTCCTAGGTCAAGCAACATTTTAATATAATCAAGTAACATTGATTTATTACATAGCCGTTTCTCAT
TTTTTTTATGCATTTCAGAGTTTTGGGGAAATCAGAACATGTGTCTCCTCAAATACTTAGCATGTGTATC
ATTAACTAGAATCCAGTATTTGTTTACTTTTCTCTTAGAAGAGCTGTTGTACAGTGAAATTTGTAGATAA
CAGTTCCATCATGTGAGTCAGCCTCCTGATAGATGATGGCCTTCCAAAACCCTTTGGTATCAAGAGCATG
ACCTCATTTCAACAAGCATCCTTGGGGCCCTTTTCAATGACCTCTACCCCAGCCCCACAGAGATAAGCTT
GTTTTATTATTTGTCCCCAGAATATATTAATCTTTCCTTAGTTTGAGAGCTTTTTATACCTGGCCTTAGG
GAACACTTAGCATACTGTCCATGTGTCTAGGTCATGGTGCATGTTAGCTAGCTCATCCCTTATTATAGAA
CGTTCTGTCTGAACAGAGCCCCTGGGTGACTTCCCTTCAATGTGAAGTAGTGTAACTGCTGGGCTAAGTA
TACATTTAGTTTATGTGGTCTGGATAGCTTTAAACTTGCAGGGCTTCCTACTTCTGTCTCCTAGTACTAG
AATTGCATGGTATTCACTATCAGACTCTTCCTAGAAGAGATTTGGTGTCACTTTATAGTCACTTGACTTT
TAGCATCTGGTATGGCTACAGTGGAGGTAGTAGCATCATGCTTTAAAAGAATCAGTCTTTACCTCTCTTA
CTCTGCCTCTCTTTGCCTCCCATCCTTGTCCTGCTAATTGCTGTTATTACCAGCATGTATCTCGTTGTTT
ATGAGCTTTTTTCACTTGGTACTCCAGATTGGCCTTAGGCCTTAAACTGGTTTTGTTTGTTTGTCTGTTT
TCTTTCAATTCTGATGGAAAATAGGTTATAGACTATAGATCCTGGCATTTATCTGTTTGCATTACTTAAC
AAATAACAGAAGCAGCTGAACTGTCAGTTCACATTTTAGTCTTATTAAATAAAGGTTGTGTGTGTGTGTG
TGTGTGTGTGTGTGTATGTATGGCTCAGAGGCCAAAGATTTGGCCAAGCTCCACAAACTATTTCTGGCTT
CCAGACCTTTGCTCTGGGTTCCCAACCTTTTTTTTCTGAGGAAATAGTTGCTTTCCTACCTTGTTCTTCA
AGCCATTTCTATTAGAATTTTAGCCAGCCTGTGGCACACATAAAAGAAACAAAATGAGAAAGCCCACAAA
AGCTAAATTGACCCAGGTCTGACCTGCCCTGTAACAGTTACTTGGTTTGTTAATTCTCCAGCGTCTTTAA
TTAGAATTTAAGTTCAATTCCCAGAACCCAAAAAAGGCAAGCATGATGTTTGTAATCTTAACACTGGAAA
GGCTCAGTTCCAGGCCCATAAGTGACTAGTGGGATTGGCATGGTGACTCAGGCCTTTAATCCCAACATTT
GGGAGTTAAAGGTAGGAGGATCACTAAAAGTCTGAAGCATCCTGGGCTACAAAAGGCCAGCTTGTCTTAT
TTATTTATTTATTTATTTATTTATTTATTTATTTTATGAAAATAGAAAAATGGCTGCAAAGGTAGCCATT
GCAAGGACTTGGTGTCAGGTAGAAACACTTAAATGTCCTTGCTACCTGATAGTTAAGCCCATTGCACATT
TGTAAATGGGTGTAAAAACACTTATAGTAATTCACACTACTAGCTACTGACCAGCCTTTCCCCAATTTAA
TCTATATTTTAATCCATGGAATCATTAAAATCTGAACTAGCCATGCTTTACCTGAACTTTGACCCACTGG
CAAGCTTTGAGGGGAAAAAGAAACCTGGAGACATACATGCACTACAAATTTGAAGTTTTCCTCTTCCCAT
TCCAGGAATCCCCTCCTTGTGCAGACAATTACCGTGTATCCGAAGCTGCTTTATTCCATAATAAGAAAGG
TGGGCCTGGATTACCACAGAAGAGGAAAACTCCTGCAGAGAATGTAGTCAAACGGCCTCGAGTACACTGA
AGTGAGCCATACACAACCACCCAACGAATGTTTTTGAGTTTAGTGTGCTTGCCTAAAATGTATAAAGTGC
ACTAGAAAATTTTCAGCTACAACCTGAACATTGCTGTGATGCATTTGTGTAGCAGTTTAAAGGCATACAT
CTGAAGGACTATGCTTCAGATTCTAATGATCCTGTAAACTGGATACCTTTAACCTGAATTTGAATCACTG
GTTATTGACTGTTCCTAATGAGGCTGAAATAGTTTTAACAAGGAGATTTGTTTCAAAAATGTCATAGTTC
ATAAAAGCAAAAGTTATACATTAGGGTTATTTCATGTTTTCCTGATTTTTATATATGTATTCTTGCCATT
TAACCTCTTTAACAGCCTGAGCAGAGAGTTTAACAATGAAGCTGGGGCTCTAAACTTAAAATATGTATAT
ATGTAAATGTGTGTATGTATAAACTTCTATCATTTGCCAAACTTGGGCACCAAAACTTTGAGATAAAGAA
TAAAAAAAAATAGCAACTTAAA

An exemplary DNA sequence of the Topbp1 gene in humans is provided below.

(SEQ ID NO: 23)

GGCGCCCGACCCCAGCCACCGCCCTGCGGCCAGCGCGTCCCCCGACTCGC
CGCCCGGAGACCCCGAGGCTCCAACGAGTTCAGAAATGTCCAGAAATGAC
AAAGAACCGTTTTTTGTGAAGTTTTTAAAGTCTTCAGACAATTCCAAATG
TTTTTTTAAAGCTCTCGAGTCCATAAAAGAATTCCAATCAGAAGAATATC
TTCAGATTATTACAGAAGAAGAGGCATTGAAGATAAAGGAGAATGATAGA
TCACTTTATATCTGTGACCCTTTTAGTGGCGTTGTCTTTGATCACCTCAA
AAAGCTTGGCTGCAGAATTGTTGGTCCTCAAGTAGTCATATTTTGTATGC
ACCACCAGCGATGTGTCCCAAGAGCCGAACATCCAGTTTATAATATGGTT
ATGTCTGATGTAACCATATCTTGTACAAGTCTGGAAAAAGAAAAAAGGGA
AGAAGTTCATAAATATGTACAAATGATGGGCGGACGAGTATACAGAGACC
TTAATGTATCAGTAACTCACCTTATTGCAGGAGAAGTTGGTAGCAAAAAA
TATTTAGTTGCTGCAAACCTGAAGAAACCTATTTTGCTTCCCTCTTGGAT
AAAAACACTTTGGGAGAAGTCACAAGAGAAAAAAATAACTAGATATACTG
ATATAAACATGGAAGATTTCAAGTGTCCTATTTTTCTTGGTTGCATAATC
TGTGTGACTGGCTTATGTGGCTTAGACAGGAAAGAAGTTCAGCAACTCAC
AGTTAAGCATGGAGGTCAATACATGGGACAATTGAAAATGAATGAATGTA
CACACCTCATTGTGCAAGAACCAAAAGGTCAGAAGTATGAGTGTGCCAAG
AGATGGAATGTACACTGTGTGACCACACAGTGGTTTTTTGACAGTATTGA
GAAAGGTTTTTGTCAGGATGAATCCATATACAAGACAGAACCTAGACCAG
AAGCAAAGACTATGCCCAATTCTTCAACTCCTACCAGCCAGATCAACACA
ATTGATAGTCGTACTCTTTCAGATGTCAGCAATATTTCCAACATAAATGC
AAGTTGCGTAAGTGAATCAATATGTAATTCACTTAACAGCAAACTGGAGC
CTACACTTGAAAATCTAGAAAATCTGGATGTCAGTGCATTTCAAGCACCT
GAAGATTTATTAGATGGTTGTCGGATATATCTTTGCGGTTTTAGTGGCAG
AAAGCTAGATAAACTGAGAAGACTTATTAACAGTGGAGGTGGAGTTCGTT
TTAACCAGCTAAATGAAGATGTAACTCATGTTATTGTGGGAGATTATGAT
GATGAATTGAAGCAGTTTTGGAATAAATCAGCCCACAGGCCTCATGTAGT
GGGAGCAAAGTGGTTGCTAGAGTGTTTCAGTAAAGGTTATATGCTTTCTG
AAGAACCATATATCCATGCTAATTACCAGCCAGTGGAAATTCCAGTTTCA
CATCAGCCTGAAAGTAAAGCAGCTCTTTTAAAAAAGAAGAACAGCAGCTT
CTCTAAGAAAGACTTTGCTCCTAGTGAAAAGCATGAGCAAGCTGATGAAG
ATCTGCTCTCTCAATATGAAAATGGTAGCTCCACAGTAGTTGAGGCTAAG
ACGTCTGAAGCCAGGCCCTTTAATGATTCTACTCATGCTGAGCCCTTGAA
TGATTCTACTCACATTTCTTTGCAAGAAGAAAACCAGTCTTCTGTCAGTC
ATTGTGTCCCTGATGTTTCTACAATTACTGAAGAAGGCTTATTTAGCCAA
AAGAGTTTCCTTGTTTTGGGTTTTAGTAATGAAAATGAATCTAACATCGC
AAACATCATAAAAGAAAATGCTGGGAAAATCATGTCCCTTCTGAGCAGAA
CTGTTGCGGATTATGCTGTGGTTCCTCTGCTGGGGTGTGAAGTGGAAGCC
ACTGTGGGAGAAGTTGTTACAAATACATGGCTGGTTACTTGCATAGACTA
TCAGACTTTGTTTGATCCAAAGTCGAATCCTCTCTTCACACCAGTTCCAG
TAATGACAGGAATGACTCCTTTAGAGGATTGTGTTATTTCATTTAGCCAG
TGTGCTGGAGCAGAAAAAGAGTCTTTAACATTCCTAGCAAACCTCCTTGG
AGCAAGTGTTCAAGAATACTTTGTTCGCAAATCCAATGCAAAGAAAGGCA
TGTTTGCCAGTACTCATCTTATACTGAAAGAACGTGGTGGCTCTAAATAT
GAAGCTGCAAAGAAGTGGAATTTACCTGCCGTTACTATAGCTTGGCTGTT
GGAGACTGCTAGAACGGGAAAGAGAGCAGACGAAAGCCATTTTCTGATTG
AAAATTCAACTAAAGAAGAACGAAGTTTGGAAACAGAAATAACAAATGGA
ATCAATCTAAATTCAGATACTGCAGAGCATCCTGGCACACGCCTGCAAAC
TCACAGAAAAACCGTCGTTACACCTTTAGATATGAACCGCTTTCAGAGTA
AAGCTTTCCGTGCTGTGGTCTCACAACATGCCAGACAGGTCGCAGCCTCC
CCAGCAGTAGGACAACCACTTCAGAAGGAGCCCTCGTTACACCTGGATAC
ACCATCAAAATTCCTGTCCAAGGACAAACTCTTCAAGCCTTCCTTTGATG
TGAAGGATGCACTTGCAGCCTTGGAAACTCCAGGACGTCCCAGCCAACAG
AAAAGGAAACCGAGTACGCCACTCTCAGAAGTTATTGTCAAAAACTTGCA
ACTTGCTTTGGCAAATAGCTCTCGAAATGCTGTCGCTCTTTCTGCCAGCC
CTCAACTGAAAGAGGCCCAGTCAGAGAAGGAAGAAGCCCCAAAGCCACTT
CACAAAGTAGTGGTATGTGTTAGTAAAAAACTCAGTAAGAAGCAGAGTGA
ACTAAATGGGATCGCAGCCTCTCTAGGAGCAGATTACAGGTGGAGTTTTG
ATGAAACAGTGACTCATTTCATCTATCAAGGGCGGCCAAATGACACTAAT
CGGGAGTATAAATCTGTAAAAGAAAGAGGAGTACACATTGTTTCCGAGCA
CTGGCTTTTAGATTGTGCCCAAGAGTGTAAACATCTTCCTGAATCTCTTT
ATCCACATACTTATAATCCCAAAATGAGCTTGGATATCAGCGCAGTGCAA
GATGGCCGGCTCTGTAATAGTCGACTACTCTCAGCTGTGTCTTCAACAAA
GGATGATGAGCCAGATCCTTTGATTTTAGAAGAAAATGATGTAGACAATA
TGGCCACCAATAATAAAGAGTCAGCACCATCAAATGGAAGTGGAAAGAAT
GACTCTAAAGGAGTTCTGACACAGACCTTAGAGATGAGAGAGAACTTTCA
GAAGCAGTTACAGGAGATAATGTCTGCAACATCAATAGTGAAACCCCAAG
GGCAGAGGACTTCCCTTTCAAGAAGTGGTTGTAACAGCGCATCTTCAACC
CCTGACAGCACTCGCTCTGCTCGCAGTGGACGAAGTAGAGTCCTAGAGGC
ACTGAGGCAGTCTCGTCAGACAGTACCTGATGTCAACACAGAGCCTTCCC
AAAATGAACAGATCATTTGGGATGACCCTACAGCAAGGGAGGAGAGAGCA
AGGCTTGCCAGCAATTTGCAGTGGCCTAGTTGTCCCACACAATACTCTGA
GCTTCAGGTTGACATTCAAAACTTGGAGGATTCTCCTTTTCAAAAGCCTT
TACATGATTCAGAAATTGCTAAACAGGCTGTCTGTGATCCTGGAAACATA
CGTGTGACTGAAGCTCCCAAACACCCAATCTCTGAAGAACTGGAAACTCC
CATAAAAGACAGCCACCTGATCCCTACGCCTCAAGCCCCCAGTATTGCCT
TTCCACTCGCCAACCCCCCTGTGGCTCCGCACCCTAGAGAAAAGATTATA
ACGATAGAGGAGACTCATGAAGAATTAAAAAAACAGTACATATTTCAGTT
ATCATCTCTGAATCCTCAAGAACGTATTGACTATTGTCATCTGATTGAGA
AACTAGGTGGATTGGTGATAGAAAAGCAGTGCTTTGATCCCACCTGTACA
CACATTGTTGTGGGACATCCACTTCGAAACGAGAAGTATTTAGCCTCAGT
GGCAGCTGGGAAGTGGGTGCTTCATCGCTCCTACCTTGAAGCCTGCAGGA
CTGCTGGACACTTCGTGCAGGAAGAAGACTATGAATGGGGAAGTAGTTCC
ATACTTGATGTTTTGACTGGAATCAATGTACAGCAACGAAGACTAGCACT
TGCAGCAATGAGATGGAGAAAAAAAATCCAGCAAAGACAAGAATCTGGCA
TTGTTGAGGGAGCATTTAGTGGGTGGAAGGTTATTTTACATGTGGATCAG
TCTCGAGAAGCAGGCTTCAAACGCCTTCTTCAGTCAGGAGGAGCAAAGGT
GCTACCTGGTCATTCTGTACCTTTATTTAAAGAGGCCACACATCTTTTTT
CTGACTTGAATAAACTGAAACCAGATGACTCAGGAGTTAATATAGCAGAA
GCTGCTGCCCAGAACGTGTACTGCTTGAGAACAGAATACATTGCTGATTA
TCTCATGCAGGAATCACCTCCTCATGTAGAAAATTACTGTCTACCAGAAG
CTATTTCATTTATTCAGAATAATAAGGAACTTGGGACTGGATTATCACAA
AAGAGGAAAGCTCCTACAGAAAAAAATAAAATCAAACGACCTAGAGTACA
CTAATCGCATCTACCCTTTAGTTACCAAACATTAAATGTTTTTAAAAATT
GAAAGCCTGAATGTGACTGTGATAGATTTGGGTAGTAATTTAAAGATGAG
TACCTGAAGAATTCTGCTTCAGAGTATAATGATGACCCTTCTTGAGTTTT
GAACACCTGAAATTGTAATCACTGAAATATTAACTGTTTCTTAATAAAAA
GTTACCTGAAATAACAACAAAATACAACTCCTCAGCTAGCTTGCTGTTAA
ACCACATTGAAGTCTGTTAAAAGATATTTATTTTTCTTGTAAATATCTGA
AGCTGTAGCTTAGTGGAAATTTTAGCAAGGTAATGGATTTTGCTTTAAAA
TGTCTGCCTTACAAATTCATAACAACAAGATTTGTCAGTCAGCATTTATT
CATGTTTTCCCTGATTTTTATCTTCTCACCATTTTACCTCTTTTAACAGG
AGCCTGAGCACAAGGTTTAATGAGGAAGCTGGGGCTATAAATATGTGTGT
ATATATGTATATGTATGTTTGTACAAATCTCCATGATGTTTGCCAAGTTT
GAATGCGCAAAACTTGGAAAATGTGACAATAAAGAATAAAAGTAGTAACT
CAAATTAGTATTAAGATGTGTTTACATAGATAAATTTTTTAAAAGAGCAA
AAAAAAAAAAAAAAAAAAAAAAAAAAAAAAA

Cdv3, or carnitine deficiency-associated gene expressed in ventricle 3, is a gene that exhibits upregulated expression in cells overexpressing c-erbB-2. The Cdv3 cDNA sequence in Mus musculus is provided in GenBank Accession No. BC096479.1 (GI:66396591), incorporated herein by reference. The Cdv3 amino acid sequence in Mus musculus is provided in GenBank Accession No. AAH96479.1 (GI:66396592), incorporated herein by reference. The Cdv3 cDNA sequence in Homo sapiens is provided in GenBank Accession No. BC007338.2 (GI:33873572), incorporated herein by reference. The Cdv3 amino acid sequence in Homo sapiens is provided in GenBank Accession No. AAH07338.1 (GI:13938400), incorporated herein by reference.

An exemplary DNA sequence of the Cdv3 gene in Mus musculus strain C57BL/6J chromosome 9, GRCm38.p1 C57BL/6J is provided below.

NC_000075.6 (GI: 372099101: c103365780-103353102):
(SEQ ID NO. 12)
GCCCACAGCGGCGGGCGGAACCGAGCGCGCAGGCGCGGCGCTGCCTCTCTCTGCTGGTGGCCCGCCCTCC
CTCCTCCCCTCCCTCCCCTCCCCGTCCCCTCTCCCCCTCCTCAGGCCCGCGCGTCTTCCTCCCGCACGCG
CCGTCGTTAGCCGAGCGCTCGCCGGTGCGCGCCTTCCTTGTCCCTCAGGCAGCGGGGCGGGCGCGACCCT
GCTCCACGCTCGCGGTTCCCAGCGAGGAACGTACCACCAGAGCACGGGGGGAGCCGGCCGCCCTGCCGCC
CGCGCCTCGGCGACCCCGCGGGGCTGAGGCGACGCCGCACCCTGCAGCCTGAGCGCACAGCCGGCCTTGA
CCCAGAGCTCGGAGCCGCGAGCGCCGCGCCCTCCGCCGGCCCCACCCATCCGGACCGCGGCCATGGCCGA
GACCGAGGAGCGGAGCCTGGACAACTTCTTCGCCAAGAGGGACAAGAAGAAGAAGAAGGAACGGAGCAGT
CGCGCGGCCAACGCGGCGAGCGGCGCGGGCGGGAGCAGCGCGGCGGCGGGCTCCAGGCCGGGCGATGGCG
GGAGCCTGGGCTCCGGTGCCAGGTCGGGCGACGGCGGGAGCTTGGGCTCCGGCTCCAGGTCGGGCGACGG
CGGGAGCTCGGGCTCTGGTGCTAGGTCCGGAGACGGCGGGAGCTCGAGGTCCGGAGACGGCGGGAGCGCG
GGCCCCGCCGGCAAGGCCATAACGAAGGTGAGGGTCGGGAGGCCGGCGGCCGGCCGCATGTGCCCGCCCC
CGCCGGCCCGGGAGGCCACGTGACTCGGGCTTCGCACGTCGGCCCGGCGGGCGGGCGGGCTCGCGGTCCT
CGGCCCCCGGGGCTCGTCTGGCCGCCCCCGTCCGCGGCCCTCTGCGCGCCGCGCCCTCTGGGCCACGGGG
GTTGGGCCGGTCCCCGGGTGACGTGTGCCCGAGTCCTCGTGGTCCCGCTTGAGCCCCGCTGGGGCCGGAT
TTCATGTTCTTAAGGTGTTCCGAGGCTCCTCTGTGCCGCCCGGGCCAGCGTGGCTTACTCTCCCAACCAC
GGCCACGCAGGGCTGGGAATTGAGTTGTGGAATTTCTGTCGCCTTCTCTCCGCCGCTCAGCTCGCCCTGG
GCTTGGGTTTAAGTAAAAGCGGCGGCAGAGAGGGCGTTTGGGAATTAGGCACGTCTCCGGATCTTGCAGG
AACGGGCTAAACTTTACCGTCCGGCCACTAGCCCCAATTTTTGAGACAAGGTCTCACGGTGTAGCTCTGG
TTGGCCTCCAGTTCACAAAGAGCCTCCGGCCTCTGTCTCTGGAGTACTGGGATTAAAGGTCTGTGTCACT
GTCTGCAATTTTGTTTTTTTTTTGTTGTTGTTGTTGTTGTTGTTTTTTGTTTTTTTTAAAGATCACATTT
ATTTGCTTATTTGTGTGCGCGCACTCCTGTGTACGTGTGCGGAGAGGTTAGAGGCCGGGGTAAATTCTCT
CTTCCCACCACGTGGGCCTGGAGAGTTAGACTCTCTGGTTCTCGGTGTTGACTGCCGCCGATTCTTAGAC
CGACTCAAGAATCCTGGCGACCACTGATGGAATTTTTCCGAGTCTGGAATCTGTTTACCAAGATCTCTAA
TTAATTAATTCGATGTTTCGCGGGTACCTTTACCAGTTTGGGGAATCCCGTTGGCCCTTGATGCCTTTTA
GTCTGAAGTGGGCTTACCAAAAACTAATCAGGTGTTGTTTCTTCCATAGGATGAAAATGAGTGGAAAGAA
TTTGAGCAAAGAGAGGTTGATTACAGCGGATTAAGAGTTCAGGCAATGCAAATAAGGTAGGATGTGATTA
CACGTGGGATTAGGTAACCTTGTTAAGTAGTGCACGCGTTTACTTTTTTTTTTTTTTAAATGCCCTCTGT
GTCATTAAATTAGTTGCCAACTCTAAAATCCTGTGGTTTCCCTATTTGCAGCTATGTGGATTTTTGGCTT
GACTAAAATGGATTTTTGTCATTTAAAAATTTTTAACACTTCGTGTTTGAATTTAGGTGGTCTTCCCTAC
CCCTAGTAGGGCTCCTGTAGTTCACTCGCTGCCCTGAGAACTTACTGTGTGACTCAGGAGAAGAAGGGGC
TTGTTTTTTTTTTTTTTTTCCAAAAAAAAATATTTGAAGCGAGTTGCCTGTGTAATACATGTCTGTACTG
TCTTCTGGAATTGGAATTACAGCTGTTAGTGTGGGTTCTGTGAGCTGGACCCTGATCTGTGGAAGACCGC
TGAGCGACATGTCACCTTGAACTCTTAATTCTAAAGTGTCCACCCAAATGTACCACCAAGCTGTGCAGGA
ACTTAGTTTTTTCTTTTACTTACTTAGTAAAATTGTTTTCTAGTTCTCTCTGGTAATTGCAGTGTAGGAT
ACTTCCAACATACACGAATTTGCATTGACCATGAAGCCACCTTGAATACCCACTACCCCAGTCAAAAAAT
TGAACGTTAATAGAGGCTCTTCTGAACCTATCCCCAAAAGTAGCTTACCCTAGCCCTCGTTTGCACAGTT
CTTAGCTGTGTATTCCGGACTGAACTTGAGCTGCTGGAATTGCAGGCCTGTGCTACCTGCCATTTCGGGC
TCTGAGGTGACTGGTTTTCAAAGTTCAGGAAAACAGTCATTTTCACAGTTGGATTGGTTGATTTAAGAGA
ACCTACATTGTCACGTTCAATCTGTGGTAGTTCATTTTAGATCTGGTTTTAAAAGTAACATGTTTGGAAA
GATTTTTTTTTTAAGTAGAAAGGGTAAGTATTTAAGGTAAGGTTTTTGAAGTTCAAAAGTGAGTAGGGCT
CTATTGCCTTAAAATCCTTTACTCATCTTACACAAGCAAAATGATTGGAAATTGTCTTTGGTTTGAAGCT
TGCAATTTGTAGAGAATTCCAGTGACCATATGGGACAGTTGTTGAGGAAATAACTTTCTTAACTCTAAAT
TAATCTTTTAAAATCTAGATTATATATGATCTGAGGTAACTTATTGAAAAGCCAGGTAGTATATTTTAAT
GTATGGTTAGTGTCTGTACCACCCAGGCATTCATATCCCTAGGTGTTCTCACTTGTGAATTTACCAAATT
AAACATTGTTTTTTATAGTAGGGAACTTAGAAGTTATCTCCCTTTTTGGTGTGGCTTTAGTGTTTTGTTT
TGCTTTGTTTTTCCTTTTTTAAAGATTTAATTACATATGTATGTCTGGGATGTATATGTACATGTATGTG
CATGCAGGTGCCTGTCGAGGCCAGAAGAGAGCGCATCAGTTCCCTTGAAACTGGAGTTACAGGTAGTTGG
GAGCTGTGTTCTAGGGACTGAATTTGTATCCTCTACAAGAGCAGTATATAAGCTTATGAGTAATTTTAGA
AAATACTTGGTGGTGAAAGCCTTAACTGTCAGCTGTTGAGAAGCTGAGGTGGGGAGAATAAGAAAAATCA
GGTGCGCTTGGGTTAGAGTGAGTTTAAGGTTACTCTGAGCAATTTAGTGAGACCCTATCTCAAAACTTAA
AAGAAAAAGTAGAAAATGTTCTTGTTTTGTATCTCCAAGTCTGATCTATAAATCTGGAGAGGGAGTTTAC
AGGTTCCTTTCTTTTATCTGGATGTGGTCCCTGCTCTGAAGTTAAATTATGCCCTTGAGTGGTAATTTCT
GGACATATTGACTTGAATTTTAGTGGGTAAAAGTAACACTTTCTGAAGGTAGAGGGGTGGCCTTCAAGTT
TTTTCAGTATAAAATTTGATTTTGTTAAATTATGGAGAACATTTAAATTGAACTGGTTTTCTAACAGGAT
ATAAAATTCTTTATTTTAACTTTTTTAGAGTCAGGAAGTTGTTAATAAACTGTTAGGATCTACTTGTCTC
TGCCCTGCAGTGTTGACATGCCTGGCTTTCATGTGGGGGCCAGGGGTTTGAACTCTGCTCTTTTTACTTT
TACAGCAAGTGTTCTTACCATATATACAGATGAACAGTTGTGATTTACTGTGTTGGTGAGTGAATAGATT
CAAAGAGTGAATGAATAACTGAAAGCAACTGCTTCAGTGTATGCAGGTGTAAATGCCATGTAGGTGGGAA
TCTTTTTCACCTTTTTTGGATGCATTAGGTTGTTGGGATATTACCACACCCAGATGATTACCTATTTCAT
CCCTGAAGCAATTATAGTATTGACATGGCTTTACAATTTAAAAAGGCAAAAAAAAAAAAACCTCTTGAAA
AAATTATGAAGATTGGAAACCACAATTAGCCTGGGTAATTATAAACTTCATACTAGCTAATAAGCTACTG
TTAGACAATGGTGTTGTGCTATCTGATCAGATTTTTACGATCCATTAGAGAATAACATGTTCCCATCTCT
TTTTCAAATGAAAGCTAGAATTTTTTACTTCCTGTTACGAAGGTGATATATGTACTGAGTGTATAAATTT
GCTCTTCGTATTGGTGATATCTAAAAGCCAGCCAGTATCATCTTGAAAGCCTGACCATTAAATGACAGAG
ATGGCAGAACCTGGACTTGAGCAATCAGTTCCTAACATTGTTTTAGCTATCAAAGTTCTCATTAAAATTA
CAGATCTTTAGGATTCTCTCTGGATGTTTGATGTTTATGATAAGTATAATGTATTAGATGATGATTTAAA
TGGCATGAATATTATTTGTTAGTGACCAGCATCATTTTATATGGCTGGACAGGTTGTATGTTAAAAGGGA
AAACTGAAATTTGGCTTTCTCTTTTTCCTTTGTCTTTTAAAAATTGCAGTTTATTTTGTGTGAGCATGCT
GTGGCATGCATATCAGAATAAAACCAACAGGAGTTAGTTCTCTTCCACCTTGTGGATCCTGGGGATTAGA
CTCTGGTAGTTAGGCTTGGTGGCAAGGACCATCTAGGTGTGACCTTTAGTTTTTGGAGAAAGTATCAGTG
ACGTCATCATTCTCTTGCCTCAGCTTGCATGCTGAGGTCACCAGTCAGCCTCCAAACGGTGGTTACTTCT
CTGTTTGTCTTTTTGAGTTACAGGTGAAGTGCTCTGTTATTGAGCCCTGAGAGACAGTCTTAGTCTTAAA
TACAGATTGGAAATTAGGTTGTTCCTCTTTCTTGACATCATAAGTCTTTAAATGCTGGGGTTACTTCCAG
CTGGTGGGCAGTGTCTTTAAAGGGCCTTAATAAAGATGGGTATGGTGGTTTACACCTTTAATTTCAGCAC
TTGGGAGATAGAGACAGGAAGATCAGTCAGGATCGTCAGCTTACTTGAGTATATAGTGAGTTTAAGGCCA
GCCTGGGCTACCTGAGTGAGGCCTTGTCTTGGATTCTAGGTATGGCTCAGTTTAGCATGCTTTTGTTGCA
TACATGAGAATCCAAGTTAGACTCCACTCACCAATGGAGGGAAAAAAAAAATCAACACTAAGCCAGAAGA
AATTTGGCTTAAAGCTAGGACACATCTTGCTTTAATAGGAATATCTTTTATGGGGGCCAGCAAGATGGCT
TATTATTGGGTATTGCTGTTAAAAGTTGATGACCTGGGTTTATTCCCTAGAATCCACATGGTAGAGAACT
GATTCCCAAAAGTTCGCCCCTAAAGAGTTTAGTCTCAGCAGAGGGAGGCAGAGACAGGCAGGTCTCTGAG
ATGTCGATCAGCTTTGCCTACGTATCTGATTCCATGCCAGTCAGGACTGTATAATTGGACCATATTTTCA
AAAGAAATGTATTTTTTTGTAGAAGTTTCATATGGTGGTGCATACATCTAGTTCCAGTTCTTGGGAAGTT
CAATGTGCTTGTTTTTCAATAGTGAAAAGGAAGATGATGATAATGAGAAGAGAGAAGATCCAGGAGATAA
TTGGGAAGAAGGTGGAGGTGGCAGTGGAGCAGAAAAATCTTCAGGTCCCTGGAATAAAACCGCTCCGGTA
CAAGCGCCTCCTGCTCCAGTAACAGGTGATCTTCTGTTTTCCCTCCTGAGGGATGGGCATAGTGGATCCA
AATGGATGCTGCTTTAGTGGAAGGAATGTAGAAATTGAGGGACATACTTTTTTTGTCCTGTAGGCTGTCA
AAGTTTTGCTTTTGTTTTTGGTGCTAGGGAATAAAGCCAAGTAGTTCACCTCCTGCTATCATCCTACCAC
TAAATCACATTCTTGGTGCTGCACTTAGGTCTTGAAGCTTACATTCTCAGCAGAAGGGAGTGAGTAAAAT
TTCTTTAGAACTAAAGAACTTTTAGGGGTTTATGTGTGTTGTTGGATGTGTGTTTGCATGTTTGTACAGT
GCTTGTGGATGGACCACAGCTTGGTGCCGGTCTCATTGTCCATTTTTATTTTTTGAGAGTAGGGTTTCTC
AAGGAACCGGGAACTCAAACCAGTTCTAGCTATCCTAGCTAGCCAGTTTGCTTTTAGGATCCTGTTTGTG
TGCCTTGCCATGGCTGCCTAGGCTTACATGGATCTGACCATCCTGTACACATGCTCGGCAGACTGTAGCC
ATTGAGCCAGCTTCCTAGCTCAGCGTGTTTGTCTGGTTTTTGCTGTGCTAGAAATTGAACTCTAAAAACT
CTGCACATGCTAAATGTGCTGTACCACAGAGCTGCATTTTCATCTCAAAATTGTTAGTTTTATTTATTTT
ATATAGCTCAGGCTGACCTTGAGTCCTCTGTAATACAGATGACCTTATGCTACCCCACTTCCCAAAACCT
GAGATTACAGGTGTGAGCTTTCCTGGCAATTTTCTAAGTTAAAAAAATTTTTTATATGTAAAGTTCTTAT
TTTTTGAGAATGCCATCAGTATATTTTTGGTAATATTCATCCCCACTCTGCTCAGGCTAGCCCTGAATTT
GTGTGTGTGTGTGTGTGTGTGTGTGTGTGTGTGTGTGTGTGTGTGTGTGTGTGTGTGTGTTTATGTCTTG
TTGTGTGGGTATCGAGGCTAGAGGTTGGCTGTGTCTTCCTCGTCACTCTAAATGCTTAAGACAAGTTCTC
ACCGAGTCTAGAGATGATTGAATGGCTGTACTAGCTAGCTAGCGCACTCCTAGGATGTTCTTTTCTCTGT
CACCACCCACCCATCCTGTGCTGTTTACAGGCATGTGCTGCTGCCATGCCTGGGCTTTGGTGCACATGAG
TTTAAGTTCTAATGCTTATGGCAAGTGCTGAAGTGACTAGTGTGAATCACCATGGAAATGCTGCCTTTTT
TTTTTTTTTTTTTTTTCTCCTGAACCTGATGTTCTTTCAAGCTACTCCTGGAATGCACCAGACACACAGC
TCCAGCAGACCCTTAGTTCTTAGCTTTGGGAGTATTTTCTTTCTCTTTCCTCTTTTTATCAGGTGCCTGA
AAAGCCAGGTGGTAGATGACTGTGGTCTCTGTTGTGCTGCTGTGGCATAGACAGTAATGTGGAATTAACA
GCAGTGAGGATAGCTGTGCTCAGTTGTTGGGCCGGATTCAGCTTGTTCACTAATATTTGGTTTTAAGTGT
TGGCTTTCTTTTTATTATTTGTGTGGTGAGTTTGTGGAGCTGGATCTTGCCACCAGGTTTGTATAAAGAG
GGTCTTTTCATATCTGCTTAATTTTACCCTGTTAATAAAACCCTTCCCAAGCCCCCTTTTAATATGTAAC
CTCTTTGGGGCTCAGCAGTTAAGAGTACTAACCTACACTCTACCAGCACCAGAATTATATCTTAGTAATT
CTTATCAGAGGACCACAAATATCTATGACTCCAGTTTCAAGGAATCCACCTTTCCCTTCTGGCCACCAGG
GGCATCTGTGTATCTACATGATGTGTGTACACATTGAAGTGTAGCTCCTTTGTACTCCTGCTGTTATTTT
TGGCATGGCACCCCTTCTCCCCTTCTCCCCTTTTTTTGTCTCTTCTAGAATATACTGCCCATTAGGTAGC
ATTTTGGGGGGTGCTGGATGAGCTCAAGTGAGAGAGGGATGGCTAGTCACATGTAGCTGTGTTGAATACT
TAGGACATGGTGGCTAATGTGACTGAGGAATTGAAACCTAGATTTCATTTTACTCATCTAAAATAGCTAC
ATGTGTTGCTTTGATCCCAGCACTCAGGACACAGACAGATGGTGTTTGAGTTGGGGCAGCCAGGGCTACA
GAGACGGGGCTGAGATGAGATGGCTCAGCGGTTAAGAGCACTAACTGCTCTTCCAAGGTCTCGAGTTCAA
ATCCCAGAAACCACATGGTGGCTCACAACCATCCATAATGAGATCTGACACCCTCTTCTGGAGTGCCTGA
AGACAGCTACAGTGTACTTACATATAATAAATAAATAAATCTTTAAAAAAACATATGAATGAATGAGTGC
ATAAGAGAGCAGGCAAGCCTCACAAGTCATATCACCCACAAACACAAATAACGAATTTTTTTTAAAAAGC
CACATGTGGTGGCTGAAATGTAAGGGTCAGTAGTTATTTAGTGTTTGGTCTGGTTTTTTTGAAGATGGGT
CTGAGTTGCCCAGGCTGGCTTTGAACTCCAGATCCTCCTGTCTCAGCCTCCCATGTTGCAAGCATGTTCC
ACTGTTCAAGGCAGGTTGTTTTTTGTTTGTTTTTTTGAGACAGGGTTTCTCTGTGTAGCCCTGGCTGTCC
TGGAACTCACTTTGTAGACCAGGCTGGGCTTGAACTCAGAAATCCGCCTGCCTCTGCCTCCCAAGTGCTG
GGATTAAAGGTGTGCGCCACCACGCCAGCCTCAAGGCAGTTTTTAATGTGTGTATCTCAAGTGCAGCTTA
GATACTCAGTAAAAGCTTGTATGCTTTCCCTCTGGCATAACAAAGCCTATAGAAAGCAATCCTTTAAACT
ATAAGAACATTTTATTTCAGCTAGTAGATGATTACAGTGCTGTCCCAGTGAGCCTCCCCAGCCTGAAACT
TTGAAATTTTTCATAAAGTGGGAAACGACACTGCTTGAATACATATCAAGGGTCAGTTGGGAAGGTAGGA
GACAGGAAAAACCTGAAACATTGTGGCCCCCAAATTCAGTCACAGTAAATTTAAAGTCAAGGTGCTTTTT
CCTGTGTTATAAGCAAATAGAGAAACTTAACATCCAATAGTCTTAATGAAATAAAGACCTTTATTATTTT
TAGGAATTTAAAATACTGCATGTGTATTTTATTAAATAATTTATATAAAATACCTTGTTTAGCCCACCTT
TCAAGTAGAGAAAGTAGATGTCACCTTTTGATAGAAACACCAGCGACAGCAGTGTTGAATTTCAGAAGAG
ATTAAGAGATGGGCAAGGTTCTACTTGAGACATTTTCTCACAGAACTTTGTTTTTATTGTGTAGTCCAAA
TCCATTCTAAATCAGGGTGGCCTTTCCTACAGTTGTTATAGGATCAGGAGTGTGGGAAAATGTTACAAGA
AAGTCAAACATTTGAATGGTTTATCTATTAGTTACAGAAACCCCGGAGCCAGCAATGCCCAGTGGTGTAT
ACAGGCCTCCTGGGGCAAGGCTAACCACAACAAGGAAAACGCCACAAGGACCACCAGAAATATACAGTGA
CACGCAGTTCCCATCCCTGCAGTCCACTGCCAAGCATGTAGAGAGCCGGAAGTAAGTATGTGAGGCTATG
AGCTAGGCTTTCTGACAGGCAGCTGGGCACATGGTGAGGCTTTGGTACCCAGGAGGGAAGGAGAAGGTGG
GGGAAGAGTCCTTTAAGTACATTATTACTGGCTTCTGTTCCTTTTAAATATGGAAACTAGAATTGTAGAA
GCATTTATCATTGCATTCACAGTAAATGTGAGAAGATGAAAGATGTTGGCATTTATAAATCTAATACTAT
AAATAATCTTATTTCATTCAGCAGGTACTTAAAATGAATACTACCTGAAGTTAAATGACCACATATGGCA
CATCTACATCTTATTAGGGACATGAATTTTCAAAAATAAAGTGGGAAGAAATGTAAATGGCATCAGATGG
AAAATTTTTTTGGGTGGTTTTAAAGTAAATCCCAGCCTTTTCACCTCGTTGAGAGCACAGCTGGCTTGAA
TTTAGACCTCTACTTGGACTTGCTTCTCCGTTCTGTCCTCACATGTGGGGGACTCTGGGAGAGCAGTGAC
TTGCCACACACATCTTTCTGTACCTGATCTAATCAGACCACTCCCCCAAAGCAAAAGGGAGGAGAAATTG
TTGAATGATGAGAACATTACTTGAATGAGGAAATTGTTGCCCTTATGCATAAAGACGTATTATCTACTAA
CTGCGAAAACTTAGAAGTGAGTCTTAGAAGGAATGTCATTATACAGCTTCTTTCTGGTTGTTTTCAAGTT
GGTCCCAAAATATGCAGAAGATGGGACCTCGGAGCTTCAGTACAGTATTACATGTTTTCTGTTCAATTGA
AGCCTTAAAATGGTTTGATAAGGATTTTTGTATTCTCTTCCTGTCTCTCCAGGATTATCCTTAGGGTCTT
TTTTTTTTTTTTTTTTTTTTTTTTTTCTTTTTAAATTCGAGGAAGAGTACTTTCAAAATTCCCTAGTGAA
GAAGAAATATTTACTGATTACATTTCTTTTCCCTTAGGGATAAAGAAATGGAGAAGAGCTTTGAAGTAGT
AAGACACAAAAATAGAGATAGGGAGGAGGTTTCAAAAAACCAGGCCCTTAAACTTCAGCTAGACAACCAG
TATGCTGTGCTTGAGAATCAGAAGTACAGCCACACACAGTACAGCTAAGGAATGGTCTTTGCTAAGCCTT
CCAAGGTAACTAGACCACAGCTAACCACCAAGAACAGCCATTCATCGTTTTGATTTCTGGATCTACAGTG
ACTGACTGAAGACCTCAAGTCCAGTGACTGTCCCTGTTGCACTATGGAAGTTCAAGAGTCACGACTGATC
TTGATGTGTGTTGGATTTAGGGGTGTTTCATTGTCTTAAATATTTGTGAATTAGATTCAACAGTGTTTCC
ATAGTTGCTCTGCGTATAAAACCAAACCTGCAGCCAGTGGTCATTCAAACATCTTTATGTTCAGATACTG
AGCCTTCGCAAGGTTGACTACCTCAGATTTGCTGCCCTTAGTTGTGGATTTCATGTGGATCACATCTTCT
ACATAAGGTTACAGCTACTAGTGTCTGTGTGAACCTTGAAACCAACTCTACTGGATTCCTGTCAGAAAGC
CTTCAGAAGTCAGCCATCTGGGTTCTGAGCCGCTGTAAAAGATGAAGAGTTAAGTGACCTTAATTAACCT
GTTCTGTGCCCCATCCTAAGGCATACTCTGTAGGCTGTGGCTGTGTTAGACTCCCTGGAACAAGCCGCTG
TCAGAAATGATGTGTTTGGATCATCTGTGTGGGGCTTATGATTTATAATTTAAGTTTTTAGTTTTATGCG
GTAACTGGATTTAATTCAACCAAACTCTGGTCCACCAAATGGGGAGTGGGTGGGGGGGTAGTCTTATTTC
TTTGAATAATTTTTCATTCAGATAGTGCTTGTTCGGTTCCACATTAAGGCCTTGCTTTGACTCAGAGTAA
GTTCTTTAAGGGCATATTGTCTGAAATATGTCTGAAAGAAGCATTGGAATTGTAAAATGCCCTATGAATT
TGCCAATCCTTAGGTGGAACCAGATAGCCTTTGATGCATATAAAGGACAGCGGATCCGCTGCACTCCAGG
TGCTGCTCAAGCCTCCTAATCAGGTCTAATTGAACTGCTGTGGACAGAGTGTGGCGTCTGCTCACTGCTG
TGGACAGAGTGTGACGTCTGCTCAGGAGGAGAGGGTTTCACTGACCTGGCTTTTAACAGTTCAAAGCAAC
AGGAGCAAAACACCCAAGTTGCGCATGAATCTGGCATGGTGTCTGATAAATCCTTGTGTGTGCCCCAAAT
GTGAAGGTTATGGGCTATTCATTGTTTAGCCAGCTCTTGACATTCACCTTGGGAGTATGTTTGGTAATCA
CTTGGAGACGCAGGGGAATAGTCAGTAATGTAGCATTTCTGTTGTAATTTCAATTTAACTATCTAGTAGT
ACTTGGCTCTTAGAAGAGGTTTCTGCTAAATTTCTTTCTACCTAATGAAATAGCCTGTCAGTTACCTACC
TTGTCACTACAGATGGTTCAGTACACATACTCTATGTTGGCATATGACTATGTGATACAATACTAGTGCC
TGGGTTTCTCTCTGTAAACGAATCCCTTTGCCAAACACAGGTCAGACTTGCTCACTACTGACAGGAGTGG
AACAGGAAATGGATGTTTTCCCAGCTTGAGAGCATGTTTAGTGGATTTAGGGCAGCAGTGTAGGAAGCAG
GAGGTGGAGGCCAGTGTCACCTGACCAGACTGTATGCCACTTCAGAGCAATAATGTTGGCATGATCTGAA
CCAGCCAGGAGGAGGTTGAGTGATTAGAGGCTTGAGATTCTTAGTTGAACTGAAGACTTCATTTGGGCAC
CAGAAACGTTAACACCTTGAGCCACAAACTTCCCTTAAAGTATGCTTCACTCCAGCGTGACACACAAACA
CAGATTCTGTTTGGGGTTATGGTTTAAGACGTGAACAGAAACATGATGAGGTTTGTTTAATGGCATACTG
TACTGGAACGCTGAAGAACTGCAGCAGATGTAAATTCCAAGTCTTGTTATTTTTTAAGATTGTGAAATTA
TCAAAATGCATATGAATCAAGTTTTAATACACTGTCTGGGTGGATGAGGCTGTCCATTGCACCATTTCTT
TCTTTGGGTTCTCAGGCATGGTTCTGACAGTGTAAGAACTCTGTAACATTTAACATTGAATAAACAGTAA
ACCAATGGA

An exemplary DNA sequence of the Cdv3 gene in Mus musculus strain C57BL/6J chromosome 9, GRCm38.p1 C57BL/6J is provided below.

(SEQ ID NO: 24)
CCGAGCTCGGAGCCCCGCGGGCCGCGCCCGCCGCCGGCCCCACCCATCC
GGGTCGAGGAGGCCGAGGCCATGGCTGAGACGGAGGAGCGGAGCCTGGA
CAACTTCTTTGCCAAGAGGGACAAGAAGAAGAAGAAGGAGCGGAGCAAC
CGGGCGGCGAGTGCCGCGGGCGCAGCGGGCAGCGCCGGCGGAAGCAGTG
GAGCCGCGGGTGCGGCGGGCGGCGGGGCGGGCGCGGGGACCCGGCCGGG
TGACGGCGGGACCGCCAGCGCGGGGGCTGCGGGCCCAGGGGCCGCCACC
AAGGCTGTGACGAAGGACGAAGATGAATGGAAAGAATTGGAGCAAAAAG
AGGTTGATTACAGCGGCCTCAGGGTTCAGGCAATGCAAATAAGCAGTGA
AAAGGAAGAAGACGATAATGAAAAGAGACAAGATCCAGGTGATAACTGG
GAAGAAGGTGGAGGTGGTGGTGGAGGTATGGAAAAATCTTCAGGTCCCT
GGAATAAAACAGCTCCAGTACAAGCACCTCCTGCTCCAGTAATTGTTAC
AGAAACCCCAGAACCAGCGATGACTAGTGGTGTGTATAGGCCTCCTGGG
GCCAGGTTAACCACAACAAGGAAAACACCACAAGGACCACCAGAAATCT
ACAGTGATACACAGTTCCCATCCCTGCAGTCAACTGCCAAGCATGTAGA
AAGCCGGAACAGGTACTTAAAATGAATGCTACCTGGAATTAAATGACTA
CATATGGCACATCTACATCTTACTTTAGGGACATGAATTTTTTAAAATA
AATAAAATGGGAAGAAATGTAAAAAAAAAAAAAAAAAAAAAAAAAAAAA

Rab6b is a small GTPase that is thought to play a role in retrograde membrane traffic at the level of the Golgi complex. The Rab6b cDNA sequence in Mus musculus is provided in GenBank Accession No. BC059903.1 (GI:37604218), incorporated herein by reference. The Rab6b amino acid sequence in Mus musculus is provided in GenBank Accession No. BAF02859.1 (GI:112292963), incorporated herein by reference. The Rab6b cDNA sequence in Homo sapiens is provided in GenBank Accession No. BC078662.1 (GI:50927455), incorporated herein by reference. The Rab6b amino acid sequence in Homo sapiens is provided in GenBank Accession No. AAH78662.1 (GI:50927456), incorporated herein by reference.

An exemplary DNA sequence of the Rab6b gene in Mus musculus strain C57BL/6J chromosome 9, GRCm38.p1 C57BL/6J is provided below.

NC_000075.6 (GI: 372099101: 103112074-103185270):
(SEQ ID NO: 13)
CGCCCTCCTCCTCTCCCCTCCCTGCTCCTTCACCGCTTCCTCCTCCTCCTCCTCCTCCTCGCCCGGCCCC
AGCTGCCAGCACCATGTCCGCAGGGGGAGATTTTGGGAACCCGCTGCGAAAATTCAAGTTGGTTTTCCTA
GGGGAGCAGAGCGGTGAGTACTCGGCTTATTCTCTCGGCTCGGAGCCGAGTCTTTTCCCCACGCCGCCCA
TTCAGCCACCCCACTAGGGACCCTCCCAGCCCTGGCTCGCCAAGGCCCTTCCACGCCCCGGTAGAGGAGA
GGGATGCAGATGGGGAGATGGGGCTGGCTCGGGTGGGGCCGGGCTGGGGAGGACCAAGGGGGAAAGGAAG
AAGGTGGTTCTGGGGGTCAAGGGATGAGGGTGAGTGTGAGATCTGGGTACGGGTACCCGGTGCAGGTGCG
TCCGGGAGGACCGAGGGCGGCGGCTGGCTCACCCGCTCTAGGCGCCGGGCTCGCGGGCTCGCTGACTAGC
TCGCACTGCGGCACAGGCTGCGGAGCACTGTGCAGGTGTCTGGGTGCCGGCGAGGGTGTGGGCGAAGGGA
CAGGAGCCTCTCTTGGGAGAACGAGGTGGGGCTGGGAGCTGGTTGCCCCCACCCCAAGGTTGCGCCCGAG
GGGCTCCCTGCGGACCCTGCCTGGAATCTAGGGAAAGGACGCGTCCAGCTCTGGAGCAGATTGCTGCTGG
GAAATGGGGAACACGACACCTGTCCCTTTTCGAAAAGAGTGGGTGCAAGTGGTAGGGTAACTGGGCACTT
AGCTTCTCTGGCTTACATCCGACTGGGCAGAAGGTTCTAGACTCACTGTCCTTCACTAATGTGTTAGCCT
CTTTGGGGCTTAGGGGTGTGGATCGTGAGAGTGAAGATTCTTCCCATAGCAGGTGACTTGGGGGATGGTA
CAGAGGCGTCTCATATCTGTGAGCTTGGCACTGGGCAGAGAAAGGCCTAGGCGACACGCCCTGCCTGGCT
TCCCTGCCCCCTCTAGAGAGCATCAGTGGAGCCGGTGGATTCTCCCACCCCCCATCTCCCAGCTCACCGC
CCTTCTTGGGGGAGGGGAGGTCACCGTGGCAGCCAGCCAGAGCCTAAGGAGGGGGAGAGTAAGGGTTTCT
TTGCTGCCCAGGAGTCTCCCCTCCCAAGTATCTGACACCCAGGTTGGTCCAAGGTGGGAAGCTCAGACAG
AAGAGCAGCGACCCCTCTGATACCTGTGGGAAGGAGTACCTCCTTGAGAGCCTCGGTCCAGGAGTCACAT
AACCAGGGGGTTACAGACATAAGCTGGGCTTCTGAGCATTTCTGCTGGAAGAGAGGGGGATCCCGGGGGG
GGGGGGGGCGGGATTCCCAGCTCTAGCTCTGTAAACCAGTTCTGTTAGCTGGTTTGGAGGCCAAGAGGAG
GAAGGCCGTGGGAGGACCTGGTGGGCCCAGGCACCGTGCAGCTATGACGTGTGCGTGTGGGCTGTGGAGG
TCACATGGCCACCTCCTGTCTGCTGATGCTCAGTACCTGAGCTGGAGGGTTGGGAGCCGGGTGGAAGTCG
GGGTTGCCTGTCCCGGAGAGCTGGGTTTGGGACATTTCACATCGTACCCAGCTCAGCTAGGTAGCCAGCT
GGATCTGATTCCCTGTGCTTTGTAGATCCCAAGTGTCACAAAGGACACCTCCGTTCCTTGCTGTGTGGAA
AGGATTTTGGGAGCACCTCAGCCCTAGGAGGGAGCCTGCAGGTGCACAGCAGCCTTGGGGGGTGGGGGGG
GCTCTCAACTGTGTTGTCATTTGCCTGATTCCAGGCCTCCTGGACACTCAGTTTCTGTCTCTGAGTAGTG
GGTAGCTGGAAGCAGCCCTTATGCCTCATGTTTCCCCCAGTGCCTGGACTATGCTGGAGTTCAAAGAATA
TTTGTTAATTCTAAATCTGAACACAGTTCCATAAAGAATCATATGTGGTGAGTTTGAATGAAACTCTGGG
CTCCCTTAGCAACTGCTGATGGGTGGAACACACACACACACACACACACACACACACACGCCACCCCCAG
TGAGAGTTCTGCCAACCCCTCCAGGTCTGGAGTCACAGAAGTGAGAGACAGCCTTTCCTCACATGCTCAT
GTGGAGAGAGAGTGGGGAGGTCTTGCCCAGAGACCCACTGTGAGTTGTGAACACTTTGGGAGCAGGAACC
CCCACTTCCATTTTCCCGTCTGCCATTTTTAACTAGAGTGTCATTGGGAAATAAGATGACCTTCAATGCG
GAAAGCCAAGTCTGGAACCTCTTTGAGAGGATTTATGGGCTCAGTTAGTTGGTTGTGGCCTGGTGGAATG
CTTGGCTAATTGGTTAGGGGCAGGATTCATAGGCCCTTGGAACATAATCCATCATTGTGTTATTAGTGAG
TTGTTTACCGCGGGTGTACAGAGCTTGTGGTGTCTGTGAGTCTCAGAAGTTATGTGTATTCCGGAGCCTA
GTAGGCCGTGGCTGTAACCCCTTCCATCTTACCTCCTGACAGTTTGCATGCCAGCATCCTCTGCAGCCTG
TGCTGTGGAGGGCGGCTCCTGACATCTGGTCCAAACCCTACCCAAGCTCTCCCTTCCTGTGTGATCACGG
AGACCCCTCACGTTCCATACCAGGGCACTTGAGCCAACTGGAATCCGAGGCCTAAGCTTCCCTATAGAGG
CCAGCTAATGGAGATGGGGGAGGGAGGCGATAAAAGAGGTTGAAAGGACACAACCTTCTCCTGATCTCTA
TTCCTGACATTGTGGTAGCATCCCTCTAGGCTTGTGGACAGAGACAGCTGGATTCTAAAGAGGGGTACCA
GGCTTGGAGCAGGCTAAGGTAGGAGATACCCTGGAACAGATAATCATAGGTAGGCATGAGGCCTTCAAAG
GTACCGAGGAACTGGTTCAGGTAGGAAGCCTTTGAGTGCCTGCTCTCCGGAACGCGCCTCTCGCCTGTGG
ACTGTAGTCTGACTGGCAGTGCCTGTGTCACATGGAGTAGGATCCTGGCCAGGTGCTGTGGACTTAGGGT
GAGGTGTTTCCTTGCGTCAGGTAGGTCATGCAATGCTGCTGGCCTTCAGGTCGCTGTGTGGCGCCTCCCG
ATTAGCCCTAACACGGGTGGCAGTGGTGGCGAACCCAAGAGGGTGGTGCACGCCAGACTGGTGTGGAGCA
TCTGAAAAAGACAGGCAAAGTCTGAGGGAGAGTGGGCCTGCGAGGTGTCCATCTTTCTCCCAAGTTATCA
GAGCCTCCCAGGGGATCTGGACCTTACCAGACACATCCTGGTGTGTGTGGTGAGGAGGGAGGGAAGGAAG
AGTGGACAGACATGGGGGGAGGGGGGCTGTCTGCTTTCCCGGGCTGAGGCAGTAGGCCATCCGTCTCTAT
AGGCCTGAGTACAAGGAGTAGGGGTAGATTTCAGAGAAACCCCTCTCTCCTTGGCCCAGCTCTGCAGAAA
GTGTAGGTAGAGGCACGCCTGTCAGGAGCGACTCTAACCTTCCGAACGCTACAGCCCTCTAATGCAGTTT
CTCGTGTCGTGGTGACTCCCAGCCATAAACGCGTTTTGTTGTTGCTTCATAACTGTAATTTTGCTACTGT
TATGAAGAGTAATGTCAATATCTGATATACAGGATAGCTGATACGTGATCCCAAGGAGGTCTCGACTCAC
AGATTTGAGAATGATGGGATTGCTGGCACCCGGTTGAGACCGTCCTATACATCCTCTTCAGCTAAGGGTT
AAGAAACAACTGTCCACAGACTGAGCCCTGTTTCCTGCAACCACCCCATGCAGCACAAACCGTCCTGTCC
TAGCTTGGTGGCAAGCCCACTGTCCCTTTTAGAAGCTTTTCTCCTTTCAACTTCCAGGCTTGTCACATGG
ACACAGCTTTGATTCTTTTAATGAACATTAAAAAAAAAAACCCAAAACCCCACAAAACTGGTTATGTGCA
GTCCCATGACGCCTCTTCTGTCTCAGCTAGGTAAGGATTTTAATTTAAGGATTTTAGGGTGAGTTTTAAG
CACCCTAAGTGGGTGACCAAAGCACGTTGAGAGGATTGTGTCTGAGTCATCTCGCTCCCTGCTCCCGTCT
GTGGGCTGTGCGTGCTCACCGAGGATCTTGATTCGAAAACGATGACATCTTTGATGTGAGAGATTTGCTG
ATGGCCTGTCAGCAGATTAACAGTGAAGGGAGGTGAGGCAGCTTTGTGTGGTGTTCCTGGCACCTGGCAC
TCTCTCAGGCCAGATTCTTCTTATCGGGGAGTTGACAGTTGCTTTGTCTTGTTTTGTTTTTCCAGAGAAT
TGCATCATTATGCCATGGTCATGCCCAAGCACCTGAGATTCTCAAAGGGTTCCCCAACTCCCTTAATATC
TCTGAAGAAATATGGAGGTGGAGTAGGGGCTCCTGTACTACCAGAGGTGGGGAAGGACACGGCAGACCAG
ACCAGATCTCTTGGAGGCTCAGACTCAATCCCCATGGTGTTCTGTGCCTTGCTTTCTCATCTCGGTTTTT
TTCCCTCACCTTCTAATTTCTCTCTCCCATCCCTCTCTCTTGGGTGCTAGTACAAGAGAGAGAACCTTGG
GCGTTGCGTATAGACCTACAACCCTTGATTTTTTTTCCCTATGTTTAACAAAGTGCGTCGGAATGACACA
GACAGTAAGATGAGGCTGTCTGTATTCCTTCTGTTACTATAATGAAACACCCGAGTCAGGCCAGCTTTAT
AAGGAGAGCAGATTTGTGGAGCTCACACTCTGGAGGGTCAAGGACAGGGTGCTGGCATTGGCTCCATTCT
GGTTGAGGCTCTCATAACAGATGACATCATGGTTGGGGAGTGATTCTGAGGGCACGAGGTCATATTACCA
CAGGAAGTTAGATAACTTCACAATCCTTCCCCAACACTGGCTCCAAGGGATCGAACCCCTCTTAGCAGGT
TTTCAGGCTTACTCTTTGGGGGAGGGGTTAGGTGAAGGTCAGGGGCTAGGAAGATGGACAAGGGTCTTAC
ACAGGCCTCACTGGCTTTGAACTCCCTGTGTGAATGGGGATAACCTTGAGTTCTCCTCCTCCTCCACCTC
CTGAGTGCCAGGGATTGAATCTAGGGCCTCATGCATGCCAGATGAAGCACCAAATCCCCTGGTCCCGCCT
CTTAAAGGACCCGCTGCCACATGAGGACTGAGCCACATCCAGACCAGAGACTTAAAGGTTAACCCTCGAG
AGAAAAATGTTGTTATTTATAATCCTCAAATGCTCATAAATCATTTTAAAATAGAACAAAATAACTTCCT
GTCCTTTGCTTCCATAGTGCACCCCTAAAACCAGTCTGTGTTGCCCTGACATTGCAGACCATCCTTAACA
GACCTGAGAGAAGCGGCCTGTAGGTTACCTCCACCTGGCTCTCCCCACCCAACTTCCGCACCCAGTGCCT
TTCACTGTGGCTTGACACAACAGGGCTCCTTGGTCTCAATATGATGACATTGTTGGCCACAACTGTCTGG
GGAGCTGAAAGGTTTCGTAGAACAGCAAAACATAAACAGGATTGAACTACTGTGCTGGACACTGTCTGTC
TGTCTGTCTGTCTGTCTGTCTGTGGTCCTTTGTTGTTTCTTGCTGTGCAGAAGAGATGATGGAGGAGCCC
TTGCTGGAGGTTGCCAAGGAGAAGGACCAGACACGCCCTGAGATGGGCAGCTCTTTTGTTCGAGCCAGAA
AGACAAGCTGCTGGGAAACAGCTCCAGGCAGGGCCTGGGACTTGAAGTCCCAAGGGGACTGGGAATGGCA
GGCGCTTTGTTTCTAGAGCCAGGGAGCCTCCCCCCCCCCCCCCATGTTGGCTTACTTCGAAGGGCTCCTG
AGGAGGTCTGTATTATAGGTCTGGGCTTCAGAAATGAAATTTTCTTCATTATTTCTATAACTTCTGCTTG
AAACTTAAGAGCGATCATTTTAAAGAGTAAAATTTTTGTTGAATACTTCTTATCACTTTAATAAAGTAGC
TTATTTTTAGAAGAAAAACATATTTATCCTGTTCTCTTGGTAACGAGACCAGTGGGAGTCCAGGTAACTG
GGGCTACGTACCTCGGCCTAGCATCGTATCCAGGGAGTGATGTACTCAAGGGACAGGCCTGGGTCCTGAA
GAGTACAGCTGACAGTGTGGCCCTGAAGGCAGAGTTGATTCTTTAAAGTGGTCTCCGAGAAATTCACTGA
AGGGCTGGTTGCCCGTGTCCTTCCTCTGTGAACCTCAGGTCAATGCCCCGGGCTCTCCAGGGGCACTTTG
TACCTTCCTGGTCACTCTGTGATAATCATGCACTGATTGCTATTTTGCGGTTCGTGAGGCTGCGGCTTGG
AAAGAGCTGGTCCTGCCTACCATTAGTGTAACAAATTAGAGCTTGGAGTGCCGGGGTTTGGGGGACAGCT
GAGTCAGGAGAGTGCCTGCCTTGCCAGCATGAATAGCTAGCCGAGCTTGAGCCCCAAAACTCACAAAAAA
AAGTCGAGTGTGGTGGCAGATGCTTTTAATCCCAGCACTGGGGTGGCAGAGGCAGGTGTATCCCTGTGGC
TTACCAGCCAGCCAACCTGGTCTACGAGGTGAGCTCAAGGCCTCCGGGTCAATGAGAGACGCTGTTTCAT
GAACCTTTGGATCAGGTCATTATCCAAGGCAGGGCCAGGAGTGGGGACAGAAGAATATTGAGGACTTAAA
AGCAGAGTCTGCCATGAATGAATGACTCAGAACACCTGTACCAAACACTTTGCTAAAGAGGAACCAGTAG
ATCACCGTGTAGCTCCACTGACCATGAAGGATGCTGGAGCCAACCACAGGGAGGACAGGCTTTGGGGAAG
GAGGCAGGCAAGCTCCCCACGAAGGAGGCTGGCAGCCCTGGCATTGTGTGGCAGGCAGTCTGTCTCCCCA
CACAAGGAATTGCTTACTTTGGATGGCCTGGAGAGAACCAATAGAGAATCAGCCTTCAGCCTACTTCTAC
TGGGGACATGCTGCTGGGGACTGTTCCCAACAAGGGGAAAACCAGTCAAATGTGGACCAAGCCCTGCTGG
GCCTTGTCTTGCTCCTGGGACTGCCGTGTCCTGGAAACACAGATGGTTTTTGCCACCTGTTAAATGAGCC
CCTGCTCCACTCTGGCTCCCAAGGCTCCTGGGTACAGTGGTTTCTTAGGCCAGATGTCATTCAGTGCTGG
CATTAAGCAAGATGTCAGGCTTATAATGATACTTCAGCCTGGAGATGAGCATGCCGCTGGCGTGAGGTGC
TGCGGAGAGGCTCTCCCGATGTGTGCTTTGGTCCATCCTGTACTTCCTGTCACCAGAAGGCACTTCTTCC
ACATACGGATGGCCTTAGAGGAAGGCAGCAAGGAAGGTACACCTCCCAGGCACTCGGTGCTCAGCACAGG
GAGAGAGCCAGAACCAAGGTGGGTGGTAGTGACCTTGCCTAAGCTCCTGCTCCGACCATCACTGTGCCGG
ACCTCCTGAGTCCTGGGCATCTGGCCCGACTCACCCCCGTCACTTGTCTGGGTGGTGTCTGGGGTGCAGT
AGAACCGTGCACACTCGTTCTCCTCAGCTGGCATGACCTTGCTGGAATTTCACAAAGATCAAACGCATGA
ACACACAGCATGCAGGTAAAGCTCGAGTACCAGCTGGCTTTGTCAATAAAATTGTGACGGAGCCTCCCTC
TCCCCCTCCTCACCTTGCAGCTGTTAGGGAAGGAGTAGAGATGCATGAAGCATTTCTGCAGGTAGGCTGC
ACCTCGCAGCCCCGGGACAGCACATCCCAGTTGACAGCTGCAGAGATGGAGGCTCTGAGAGGAGGGGCTT
CTGTGTGCCCAGCAGAGCCCAAAGGCAGACCCCACTCCGCAGGAGAGAGACTCAGGCCTCTGGCTCAGTG
CTTGCTTGCTCCTGCTCATCGCCCGCAGAGGCGGGAGGCAGCACTGATATACCAGTTTCCCCACGTGAGG
GCTACCCATACCCTCATGAGCACCTCATTCTCACTGGCAGACCACAGGGGCAGAAGCTGCTCTGGTGTGA
CCTAGACCGTTCAGATCAGCCTAAGGTAAGCGGGAAGAAGCCCTGGTCCTTATAAATAGGAAGCATTAGA
TTCAGAAGCAGAGCTATGGCTGTGGTCTGCTTGAGCAAGTCGTTTTATGGTGAGTGGGAAAGGCCTGTAG
GCATGGGGATGCGGCAGACACAAGGGCCCCCTCTGTGCCAAGGCAGGAGAGGGCTTAGGGCCCCTGAAGA
ACTGAACAGGTTCAGGGCTGCTGGGACTCTAGTGCCCTGCAGGTGTGGGAGAGTAGCAGGTATAAGGGTT
GAACCTACAGAAGATGGCAGGGCCAGCCCGAAGAGGCCTCCTTGCCTTCCCACACTTGGTGTCTGAGCCT
TTCCTATAGAGAGTGGTGACCCCTGGAGGCCGGGACAGGGGAGGCCCATGCTCATGGATTAGGAGACAAA
GGCAGAGTGTGGCATATTTGAGGGTCCAAGTGACTGACCTGGGGTCAGACAGGGGGCATTTGGGGAAGTG
TAAGCCCAGCTTCTGCCTGGCTCTCTGGCCTACTCAGTTGGGTGGGTCTGTTCTCTCAGTTTGGTCTGCA
GAGACAAGACTCAAGACAATGAGATGAATGAGGGAGGGACTCCCAGCTTTCTCCATACTGCCCTCAGATG
TCCTAGAACCAGCCCCTGGGAGCTCCCCCATCTACACTGCCTCCACAGCAACCTGGGGTCCCTGGATGCT
TTGGCCTTGGTGACATGGGTGTGACAGTCTGCAGCAGTCCTTCCAGACTTCCCTCTGCCCGTTGCTCTGG
CAGATGTGGCATCTGATGCAGTGACGATCGGGCTCTGATTAGCCTCCTGCCGTTCCTCCTCTTTTGGGGG
CCCTTGCCACACACGTCCTTTGCCTTCTCCTGTGCTGCTTTCCTTTAAGGTTAGGAGATTTGGTCGCACA
GCGCAGATGAAGAGAAAGTGCTGTTGTCTGCCTTGACGACTGAAACTTAAGCTTAAACTGGCTGCTCCAA
AAGCCAAGGTGTTTGAGAGCCAGGGACCAGAGTGAGTGGAGGCAGAGGACTGCCACTCTTGAACCCACCC
CTAGCTGAGAAAGAGCAGCCTATTGGGATCAAATGAGAACAGGTTTTCAGATGACCCATGACCTCTGCTC
TCTTCCCTTCCTGCCTCAGTAGTCTTCCAATGCAACGGAGCACCGCCTGGTATAGCTTTGGCAGCATGGC
TGTAGTTCTGATTGGTTCATTTACAATGACAGCATCTCATAGTGACACTCGCCCCTTTAGCAGCACCAGA
TCTATCATCTCTGTGGTCAGGGTGGGTGAGGACTCTTCTCTGTGCTGGTGTTCAGCAGGGGAAAGAGGGA
CAACTTTGGCCCCTTCCAGCCTCCCCTGCAGGTGTGGGAGCTGGAGACCCATCCCTGGTTCTGGAAGAGA
TTGCTGTAACTGTGACCAGCGCACTAAAGCCAAGTCCCAGGGGACAGTCAGATAAGCTTCTGGCTCACTC
TGAGAGTCCCAGTGAAACATCCCACCTTGACCCTGTGGTGAGCAGGAAGTAGGTCATAGGACAGAGCGGT
GGAGCCATTTAGGACACGTTTCTCATTTTTTTCCCCTCCTGTCCAGTATAGTGAGCAGCCCCGGAGGGAG
CCACAGGTGTAGCCAGAGGGAAGAGAGACCACGGGTGTGCTGAGTCAGGGAGAGTGTGCTTCCTGCTTCC
AGCACAGGCCAGGCTCTGCGGCCACACTAGGTGTGCCTTCAGGGCTTGCTGGCTTCCTGTGAGGCTTCCA
GAGCCAAGATGCTTGGTGGTTCTAACGATGCTATCCATCCTGGGATTCTGAACAAAGTCCGGTTAAAGAC
TGGAGGTTCTGTCAAACTGATGCTGATTCCTGGGCTTTGTATGTTGTGACTGTGAAGGGCAAGAAGAGGA
GTGGGGCAGCAATGGAAAGGAGGACTCTGCCTGGTGCTCCCTGGAGCACCCCCACTGCCTGAGCACTCCC
TGTGTGTGTTGCACCGTGGGGTGATGCTAAGCCTCATCTCTCCCTCCTCCAGGGGTCTGCGCTGCTGCCG
CCGTGACCCTGCAGATGTACACTTATCTGGCCACTGTAAGAGAATGCTTCCAAATTGTTTTTAAACCCTC
GGCATATCCTGTGTGAAGCCCTAGAACCTGTAAAGTTCTGAATTCTGCCTCACTGGGTTCCCTTCCCCTT
CTCTACACCCTACCATAACACTGCAGGCCACTGCCCAGATCACAAGGAGCAGGAGGCCTGACAAGGCACC
TGGCCAGTCTCCCGTCCTGAACCTTTCCTAACCACATCAGAAAAATGCCAGAGCCCCACCTGTAGAGCCA
GGAAAAGTCTGCTCTTCTCTAGCTCTGAGAACAGAAGAGATGCTAGAGCTGGCATCATCTGGTGTCTTCT
GGTTGGGGGGGGCGGGTATGGCATGGGGGTCTGGGCAGAGGGTGACCAGTGCCTGGACCAGTAGACCCTT
CAGAGAAATCCTCCATTCTGCCTTCATCCCAGCAGGCTGGGAGAAATATCTGCTGGTTAGGAGAACTTGT
TCCAGGGGATCAGTCCAGATCACAGCAGTAGGAAGGGGAAGGGAACCACTTGGTAAGTGGAAGGCAGCCC
GTTACTGTGAGCCCCATGTTCTCAGATGTCCTTCTCCTGAGTGCAGATAGGACTTGGGACTTGCTGCTAG
CCCGTACAGTATGGCAAAGGTCGCGGGAGGTCACTCATTTAATTGTGTCATTGGGAAAGGTAACAAGATA
TTTGTCCCTGGATGGTTTCATTTTATTTATTAATTAAAATTTACTCTCTAACAATTCCACATGTGTATGC
AGTGTACTTTATTCATAACCACTTACTTTCCCAACTTCAGCCTCCCCCTCCCCCCCTCAGGACCACGCCA
GGTGACATCGTCCCCTGCTCGCCACTATATTTTTTTGAGGCAGGGTCTCCCAGTGAACCTGGAGCTCACT
GATTGGCTAGATTTGCTGTTCGGTGAGCCCTGGGCAATGTTCTGCCTTTGCCTCCTGTGCTATGATTTAT
AGGCCACCGATAGCTTTTCACACGGGAGCTGGGGATCCGGTCTCAGATCCTCATGCTGGTGTGGCAAGCC
CTTTCTCCACTGAGCCATCTCCCCAGCCCTTCTTGGTTGGTCTATTAGAAAACTGTCCCTCCGTGCTTCG
GAGAAATAAGCTGCCATGCTTTGGATAAGTGTGTGTAGAGAGGGGCTGGAAACTGTCTGTGGCTGACCTC
GAACAAGAAAGTGTCTCCAGTTTTATAAGCTTGGAGGAGGACTCTAACTTCGGAAGAGAAATACGGTCTG
GCAGACACCATGTTTGTGATCCCGTGAGACCCGAAGCAGAAGGTCCTTCAACCCATGTCTGGACTCCTAG
CTGGCAGGGACTGAGAGATCATAAAAGTTCACTGTTTCCAGCCATTGTAACAATTACCGATGGCAGTCGG
CACACAGAAATAGATAGTTTCTACAGAGGAGAGAAAGATCTTAGGGTGGGGAGATGCCTTTATCAGAGAG
GGAGTGGCATTCTCAGGAGGGACCCCTGACCAGCCCTGAGTCAAGCAGCTACCCAGGCCCAACCTGTGAA
CCCCTGATGTGTGATCTGCTTATGTTCTTCATGTGTTCCTGTCTTAGTCGGGGTTTCTATTCCTGCACAA
ACATCATGACCAAGAAGCAAGCTGGGGAGGAAAGGGTTTATTCAGCTTACACTTCCACATTGCTGTTCAT
CACCAAAGGAAGTCAGGACTGGAACTCACGCAGGTCAGGAAGCAGGAGCTGATGCAGAGGCCATGGAGGG
ATGTTACTTACTGGCTTGCTTCCCCTGGCTTGCTCAGCCTGCTCTCTTATAGAACCCAAGACCACCAGCC
CAGGGATGGCACCACCCACAAGGGGCCCTCCCACCCTTGATCACTAATTGAGAAAATGCTTTACAGCTGG
ATCTCATGGAGGCATTTCCTCAACTGAAGCTCCTTTCTCTGTGATAACTCCAGCTTGTGTCAAGTTGACA
CACAAAACCAGCCAGCAGAGTTCCCAAATAAACCTTGATTCGTGGCATTGTTCCCTGTCAGATGCAGGAC
TAGAACATTCTGTAGGGCCTCTGTCATGTCCTTGTGTTCTGACCCCACTCTGATCGATGGCCCTCCCAGG
AACCAGGTTTATTGTCCCTTAACCTCTCTGCTGACCCTTGGCACTTCCCATTGCATGTTTACAGTGGAGC
CTTAACAAGTGAAGTCTTCCTGGTCTGGTGACACCTTGGTGACCTTGTGTTTTTTATTTTTATTTTTTTC
TTTTTGTGATTTTTCAAGACAGGGTTTTTCTTGTGTAGCCCTGGCTGTCCTGGAACTCACTCTGTAGACC
AGGTTGGCCTTGAACTCAGAATCTGCCTGCCTCTGTCTCCCAAGTGCTGGGATTAAAGGTGTGTGCTACC
ACTGCCCGGCGACCTTGTGGTTTCATATCAGCAGCAAGGACAAGAGTAAAAAGAAGAGCTTCGGAGGGTG
GGTCTCAGTTGTTCATTGGAAGAGACAGTGCCTGCATGTGGCCCTGGTTAGACAGGTGGCTTCTGAGGGT
GATTTCTCCGTTGTGCATTGGAAGAGATGGTGCCTGGATGTGGCTTTAGTTCCGTACCCCCCATTCCTTC
CTCTGGCTTCACAACTCTCTGGTAAGATCTGCATCAGAGTCTTCTGTGGTTTGGGCCATTATGGGGCTGC
TTTTAGGAATGGCTCCTTAGAGACCTTGAGCTTACAGAGTCAGAAGAAGACTAAACATTTGCTCTGGTTC
CCCGTTTCCCCATCCAGTCTAGTGCCGGACAAGCAACCATCTGAGTTCCACAAAACTGGAGGAGACACAG
CGATCCTTCCTGTATCCCAGATGGCCGTTATCCACTCACAGGCCCAGGGCCCTGAGGTCTCCAGTGTGTG
TTCCTTCCTCATGGTACTTTGGCAGGAGGCCTGCCTCTATGCAGGTGTCTGAGGAGCAGGAGATGGTCAA
TTTTGTCTGAATGGGAGGTCTAGAAGTTTCTCTCTTGCTCTTTTTTTTTTTTTTTTTTTAAAGATTTATT
TATTTATTATATGTAAGTACACTGTAGCTGTCTTCAGACACTCCAGAAGAGGGCGTCAGATCTTGTTACA
GGTGGTTGTGAGCCACCATGTGGTTGCTGGGATTTGAACTCCTGACCTTCGGCAGAGCAGTCGGGTGCTC
TTACCCACTGAGCCATCTCACCAGCCCTCTCTCTTGCTCTTGTGTTCCCCTTTATAGCTGGCTTGAGCAA
GGTAGGCCTGAGCCTCTATCCACCTCCTCCTGGGCCAGCTGAATGTTGAGCTGCTGTGTGAGGGTCTGTA
GGAGGACAGGAGAAGAAGCAAGGGCCCACAGAGGCAGCATCAGAAGCAGCCCCCTGTCTGGAGACTAAGA
GCATCCCAGTTTGCCTCTTGGCACGATGCTCTACCCCTGTACCACAGTTCCTTTGAGAAGGGAGTAGACT
TCTGGGAAGGTGTTTGCCAGCATCGGTCTTCAGGTCCCTTCCACATCCCTGAGCAAGCTTTATCCCATTG
TCATGTCAAAGTCGTGACCCTAGTTTGAGGAGCTTTAGGTAGTCAAAAGTCTCTCTGACAACTTAGGATG
CATAGCCATCCATGCACTCTGTCCTCCATCTTGCCACCTCACCCCTTTCTTGTATTCCTCAGACTTTTAG
CATCCACAGCACCAGCCTTGGGAAGAAAAGGGTGAATATGCCCATTACTTTGCCAACATTATGGCTGGAT
GAGCCAAGACACCTATTAGATAAGCTATAAGCTTGCAATAGCAGGAACTTACATTATGGCTGGATGAGCC
AAGACACCTGTTAGATAAGCCATAAACTTGCAATAGCAGGAACTTCCTAAGGGGGCAGTGGGAATATAGC
ATTGCTATCTCTCTTCTCTGCAGTATGTTGTGTTCGTGGTGAACCAGTTCTGCACCGCATGGATGTGTGG
ACACAGTTGGCCTGGAGGGGCTGGCTGGAGTTGGGCTGAGCCTCAGAGCAGTTGAATGGGAGCACATTTG
CCCATTGCAGTGTGACTTTTCCTGGGAAGCCCAGGGTTGCTATGGGTACGGTGGATGGGGACCTGCCAGT
GTGGTGTGGCTGGCACTCAGATGTTTTTACTTGGCCCCAGAAGGAAGACACCATACCACTCTGAAATTGC
TATGAGTGTGCAATGCAATGAGGCAGAAAGGATTCAGACCTGGGTAGGTCAGCTGGGAGTTCTGCAGCCT
ACAGAGGGTGCCAAGATCTCGGGAGGGTCTTAGAAACCGTGAAGGTGATGTCCTTGATCATCAGTCCCAG
TAGCGAGAAGCCCAGGGAAGTCAAGACGTTTCCACATCGACCCATTGTATTGGCCATATATGTGCATACA
CTGTCTGTATGTGTACTACATACATCTCAAATGTGCATACAGCAGTGGCACCCTGTGATAAGAGCTCCCA
GCAGCCACTGACCTGGCTCAGTCCTGTTCGTCTATGAGGCTTGAATAAATGTACAGTAATATTCCAGTTC
CTTAGGAAAATTTCATATTCATCAGGGGATATGCAGTCCTTAACAGAGTGAACAGTTCTTTCAGAGGACC
CAGGTTTGGTTTCCAGTGCCCACATTGGGTGGCTTATGACTACTCCAGGGGTTCTGATACCCTTACTCTG
GCTCCGGGGGTTCTGATACCCTTTTCTGTCCTCCTCAGGTACTTCACTCACTTTGAGTACATGGCCCCTC
ACATACACGTGTACTGAAACATGTACTTAAAAATAAAATCTTACAATTTTTATATTCAGCAGCACATTTG
TTTAATCAGACAGAATGCACCATTTCCTAAATATATCGGGTATCTCAGAGACTGTGTGTATATAAGCATA
TATGTTACATATTAATATATAATATTTCCCCTTTGCTGTTCATTTTAGCATACTTTTAAGCTTATAAAAA
AGTTACAGAAATAATACCAAAGCTTCCCGCCTGCCTTTTACCCAGGACTCCCAATTTTTTAATTTTTACA
ACTCTCACAGAGTTCTCCAGACTTTTGTGAGATAGATATTTCTCTCTCCATTGAATGTCTGAGGAAGCTG
AAACCCAGAGAAGGTGGTAACTGACATAAGATCACACAGCAGACCAGAGGCAGAGGCAAGATTAAAACCC
AAGCCCGTTGGTCCTTAGAGCATCTGTGTGAGCAATGAGGCTGTGTGACTCCGTTGACGTCTCCTCTTCT
GCCCTGTGTTTCTTTTCTTGTACAGTAGATCTGCGAGCAGTGAGTTATCTCATCTCATCTGTCTTTATCT
CACTTACATTTCTGAAATGGTTTTTTTTCCCCCTGGGGGTAGAATCTGGGGCTGCCGATATTTTGAAGAT
GCCCCTTGCTCACTCTGTCATGGCCACTGGAGCCCTAGGCCAGAGGCCAACTCTGAAGGTTCCAGTGGGC
CAGGTCCCTGTCACTTACTCCAAAGAAGGCAGGTGGGATGGCGGAGATGCAGCAAGGAAGGATGTTAGTC
AGCACTGCTGTGCGGGGAAGCAGCCTTTGTTAAGCCTGTGCAGTCTGTCTTCTGGGGACGACTCGGGCTT
TAGGTTTAAAGAGAGGGAGATTGGGAGATACATAAGTAAGCAGGCTTGGTTAAACTGGTCTAGGTGATTA
TATCAGTTCTGGCTCTGCCAGCTGACTGAAGTCCTTCAAGGAGGCTTCCAAGGGAAGGGACACTGCAGGC
AGGCATTCTGTCCCCAAGGTATCCAGGTGGTGGCAGGTTTAGAGCCGTGAGTGGCTACTTTCTGGTACGT
TGTGTTTCATTCACTTTGTTTGGGGTGGGGGAGTAGAGTGTCTTGAACAGAACTTTGACAGGAAGATGTT
TTTATCTGCATGCTTTCAGGCTTGAAGAGAGCGAGATACGTCACTGACGTTTGCGCAGACCGCCCCTGAG
TAACTAACATGCCTATGTGCAGAGTTAGGCAAAGCCGGAACTTGAGTCGGAGCAGGACCTCACTGTGTAA
GAGACCAGCCTACTTCTGCCTCCAGAGGGCTGTTGGGATTAAAGGCGGACACCAGGGTATTCAACTCTGA
CCAAGTTTAAGACCAAAGAGGAAGATGTGAAAGTGAAGGCTTTTCTCCTGCTTTAGTTTTAGTCTAGTTT
TTGCCTCCTGGGGCCAGTGAGGAGCCAAGTTTCTTGGTAAACGCAGTTTCCACCTGCCTGTTGTATCGCT
TCAGAGTAACAAGGACTCTCATAGTTCTCTCAGGTTTATTGTTATTTTTGTTTAAATAGTTACCTTTAAA
GCCACTTAAATGACAAAAGTCTTGGTATCTGCCCACGATTGTCGATTGTCCTTAACTCTCTTTTGTGTCT
GTGTGTTTCTACCTAGTGTCATTTCCTGTATTCCTGAAGAATTTCCCAGAACAGTTTTTGTCATGAGGAT
CTGCTAGTGATAAATTCTGTTAGCTTTTGTATGTTTTTAGATAGATAGGTAGGTAGATAGATAGATAGAT
AGATAGATAGATAGATAGATAGATAGATAGATAGATAGATAGATGGATGGATGGATGATAGACAGAGAGG
CCTCTTTATCGTGCCATTGCTTTTAAGGATATTGTCCCTAGGTATAGAATCCTACGTTAATATTTAGTGT
AAGTTTTTACTTTCAGAACTGTTTTTTTTTTAAACCTTAGGACTGGGAATTGAATCAGGGCCTAGTGAAT
GTTGAGCACTCCGTTGTCTTCTTGCTTATATCGATTTAGGGAAGAGCTCTGCTGCCCCCTTGTCTTCGCA
TCTTGGTGTCACCGTCCAGCTGGCTTGAGCACCAGAGGCATGATTGGACAGGTCAGAGTGGAAGGCTGGA
GCCTCAGACTTGGAGCAGTAGCCGGAGTCCCATAGGGGATGTGCCCCACCCCCCAGCCCAGGCCCTGTGT
ATACTTTCAGAGATGTCTTAGGGTTTTGCTGCTGTGAACAGACACCGTGACCAAGGCAAGTCTTTAAAAG
ACAACCTTTAATTGGGGCTGGCTTACAGGTTCAGAAGTTCAGTCCATTATCATCAAAGCAGGAACATGGC
AGTGTCCAGGCAGAGCTGAGAGTTCTACATCTTCATCTGAAGGCTGCTAGTGGAAGACTGACTTCCAGAC
AGCTAGGGTGAGGGTCTTAAAGCCCATGCCCACAGGGATACACCTACTCCAACAAGGCCACATTTCCAAA
TAGAGCCATTCCCTGGGCCGAGCACATACAAACTATCACAAGAGGTTTCTGCTAACCACTGGCTCTGTGC
AGCTGGGAAATGGAGCTTCTTGGTGGACTCTTCTCCCCCTCCCCTCCCCTCCCCTCCCCTCCCCTCCCCT
CCCCTCCCCTCCCCTCCCCTCCCCTTCTCTCTCTCTCTCCCCCTCCCCACTCCTATCTCACACTTCAGCC
TTTGAGACCTCCAGTCACAGATAGAGAAGACACTGGACACTGCCCTACAGCTAGCTCCCTGGTATTTTGT
TCATGTTGATTCTTTTATAGCTCATCTTTGATAGTGTTTATCACTGTTCGAAAGTTCATTGCTGTTCTCT
TCTGTGATGTGCAATCATCTTTGACATCATAGTTTTCACTGCTGGGATTTTGATTTGTGTCTTTAAAAAC
CTTTTCCTTGTTTCTGTTTAATTTTTTGGACATATGGAATGCAGCAATAACAGTCTCATACGTAATGTTG
AAAGACCCTCGTTTCTGGGGAGACTCTTGCTCAAGTCCTGGGATGAGCTGACGCCCCAATAACTCGAGAG
AAACCGTTCTTGATGCAATATCACAGGAGGTTTATTTATCAGCATGCTGAGGCTGAGCTCGTAACCCACA
CAGGGGCAGAGGAGATGACCCCCAGCAGTTGCAGTCGAGGGTTTTTAAAGAATAAACTGCAAGGTGAGGG
GAGGATAGGACCACAAAGAATGGGGAAGCTGAGTAAACACTGAAAAATGGGGATGGTGAATTACGGCTCA
TCTCTCACGGAAAGGTTACAGGCTATCTTTGGCAAACATTCTTGGTCCCTTACACTATGGGTCAGGCACC
TGGGTGGGTCACCCAGTGGTCATCATTCTCTCAGGCTGAAGGCGAAAGAACAAAGAACTCCATGCTAGGC
TTCGTTTCTAGGGAGTCTAAGAAACACATTGAGTTGGGGGTCTTACAATGTCTTTATCTGCTAATTCTAC
TGCATCAGTTTGGGTTTGGTTCCAGCTGATTTTTGTCTCTCCTTTTTTTCCCCCCAAAAAAAGATTTATT
TATTTATTTACTACATGTAAGCACACTGTAGCTGAGTTCAGACACTCCAGAAGGAGGCTTCAGATCTCAG
TATGGATGTTATTGAACCAGCATGTGGTTGCTGGGATTTGAACTCCAGACCTTCAGAAGAGCAGTCAGTG
CTCTTACCCGCTGAGCCATCTCACCAGCCTTTCTTCTCTCCTTTTATGTTCCCATTTTCAGGCTTCTTTG
CATGCCTGGAGTTTTGGGTTTTTTTTTTTTAAGGTTTTAATTTTTTATATGTATGGGTGTTTTGCCTGCA
GGTATGTTTGTGTACCACATGTGTGCCCAGGACCCAGAAGATAGCGTGGAATCCCCTAGAACTACAGTTA
AAGATATTTGTGGGTTCTAGGAATTGAACACAGGTTTCCTGGAAGAGCAGACAGTGCTCTTAGCCTCCGC
ACAACCTCTCCTGGTGATTTTCTTTTTTAAATTGTTCAGGTTTATTAATTAGAAGGTGCCATATCCTGAC
TCTGTCTTCAACAATTATATTCCAGGCTTCTTTTCTTTCTTTCTTTCTTTCTTTCTTTCTTTCTTTCTTT
CTTTTCTTTCTTTCTTTCTTTCTTTCTTTTTTTTTTTTCTTTTGGTTTTTTGAGACAGGGTTTCTCTGTA
TAGCTCTGGCTGTCCTGGAACTCACTTTGTAGACTAGGCTGGCCTCGAACTCAGAAATCCACCTGCCTCT
GCCTCCCGAGTGCTGGGATTAAAGTCGTGTGCCACCATGCCAGTCTTATTTTCGAGAGTTATGAACAATC
TTTTTTGGCACAGGGAAGGAATTATCTAAGTAATTAAATAAGGGCGGGTACTCCTGGCACACTGTGGTTC
TAAGCCTGAGAATACAACTGTTACCCTCATGCTGGCTACCCCGGCTCTCCTCTGCCTTCCCACCACAGTA
AGCAAGCAGCCAGACCTTAGTTCTGTCCTTTGGCTTCAGGACTCTGGGCTGATGGGAGGAAGTGGGACGT
CACTAAGATTATGAAGCCCTGGGGTAAGAGGATTCTGCCCACAGCAGTAAAGGAGTCTTTTCTAGAACTT
CCATCCCACAGTATATTAAAGTCAATTTGGGTCTTAAAGACCATTTGGCTCAGCCTCAATGGAATACCAA
TGAGCCAAGGCAAAGCCAAGACGTGTTCAGGGCACATTGGCCAACTTAGGCCTCCATCATTTGACAGGCA
GCTCTGGCTGGGTCTTGATGATTGAGCAAGTCACTGTTATATCAATGAACCCATCTCACTCATGAACAGT
GCCTTTCTCAGTTCTGGACTGTTTGTCCCACATGTCTGTTGTGGGACACAGTGTCTTTATTTCGGTAACC
TGAGGCCTTACTCTGCAGAGCCACCTTCAGGCAAGCCCGTCCTGTGTTTCTAGTTCCTGGTTTCCCTTCT
TTAGCCCTTGACCATGTGATGTCCCCTTCCCTTTTGTTGTCCACGTCTTTCAGTCACTCAGTCCTGTACC
TACTACCACCCTGTATGCCTCCTGACCTCAGGTGATAGAGGTTGGGGTCGGGGCTCTATGTCATTCCCTG
ACCTTGAACTGAGAAAGCCTCAGGTCAGAGCTCAAGACCTCTTCCTGATCCTGGCCTGAGGTTGGCTTTG
GCTTGTTTTAGTGTCGTAGGGTTAAGGAATGGGACACTCCTGTAGCTCATGCACCCCAAATCATTCATTC
TCACAGTGGGGCTGGCCAGGAGTAATGCCCGAGGTCTATTTAGGATTAGGGGAGAAGAAGAAATCTCAGT
TAGGTCTGGGTGGGCAGGGATGATTTGGTGTGAAATCTCTTGAGGCTGTGGGGAAGGAGAGATAGAAGCA
AAACTAACGGCTTGCGAGCTCCCAGAGGGCTTTGGAGGGTAAAGAATGGACTCAGAAATGTGAAGTACTG
CATAGTAGTCTGTGTGGGTACTCACCCCACTTGATGCTGGGTCTCCAGCGCTTGCTTTATAGCCCCCAAG
TTTGTACCTTTGACTGGCATCTCCCCATTTCCTCCGACCGTGTGCTCCAGCAATCACCTGCAATTCTTTG
TCCTCTGTAGATTGAACATACAGGTGAGACCATGTGGCTGTGCTGTGTCTTTTTGTGTGCACAGTATTGT
CATTACAAAAAAAGGGTATCAGTGGCCCATGCACACACATAGGCATGCACGCACACACATATACCACAGG
CACACGTATACACATGCCAGGAAGGAAGCTGTGTTGTGATGAATACATCAGCTGGATTAACTGCCATATC
CCTGCGTACATATCTATAGAGAGAGGGAGTTCTGTCAGAGCATCACCTCCTATATGTTAACTCTTTATAG
CATTGACTCGTCAGCTATGCCTCCGTACACCTGGGGGTGGGGAAAGAATGGCAGCTGGAGAACACACGGA
TGAGCATCATGCAGGAAGGTGGCAGAGCCCTCCCTCTGGGCAGCCGGAGGCCCGGGTTGGGCACCTTTGG
AAGAGTCTCACCTACCTCAGCCCTTGGGCAGCTCTTCCCTCCAATTAACTTTCTACTGAAGGAGGAGCCA
CTGGAAAAGAAGCAAGACAGCCAAGAGTGTATGTGTAAAATGGGAAACTGAGGCTGGGCCTCCCAAGGCA
CTGTCTGGTGGCCGTGTGCAGGCTTAGCAATCCATTGTCATTATGTCACAGAGCTTTGAGGAGGGTGTCT
GGAAGACTGACCACAGCATACCTCAGGCTCCTAGACAAGGGAAGCAGGCTTGTCTGTAGTATTAGACTTT
GTGGGGAACAAGGAACTTTTATTTTGCCCTTCTGGTTTCTGCCTTATCCTGTCTACATGGCTGAAGAGTG
GGTTTCTTTTTTATTTGACAGCATTTTATTTTTAATTCTTCTATTAATTCATCATCACTATTGAAATTAT
GTATATGTGTCCAAGAAGGCCAGAGGTATCAGATCCCCTGGATCTGGAACTGGAGATAAAGGTAGCTGTG
AGCCACCGGACATGGGTGCCAGGAACCAAGCTCCGGTCCTCTGCAAGAGCTGTAAGTGCTCTTAACCCTA
GAGCCATCGCTCCAGCTCCTGAGAGTTGAATTCTGTGTTGCTAAGCTCTTTCTTTGGATCCCCTGATCGA
TCCTGCTCTTTCTTGCTCTCACAGAAGGGCTGTAATCCTCAGGGATTGCAATGGCTTCAAGGTCAACACC
CAGTCAGGGCCACATAGGAGGCCAGAACTCCCTTCTGTTGGCCTGGATCCCCACTTCTCCAGCCAGCACA
GAGGATGGTAATGAAGAGACATCTGAGTCAGGACAAGCTTCCAGAACACTGGGCTTAGAGGAAGTGGGGC
TGTGGGGGTGGGGTGGGCGGGGAGACTGAGGCAGGAACACTCAGAAGGGCAGAGGCTCAGGCATGGCCAG
CTCTGGCTGGTATGGAAGGGCCTAAGCCACACTTGCCTGCCCCATGCCTTGCTACCCTCAGCCTCCCTCT
TTGTCCTGCCCCCCATCCCCTTGTTCCATCTTTACTGGCCCCCTAGTGAGGCCCAGGCAGCTGAGCATCC
GCTGGTGGAGGGAATTAACATACTTAATTAAAGTGTCCACGAAGAGCTTTGCATACTTCTTAATTACTGT
CTGAGTCTGTGGGGGGAAGGTTTGGAATGGGATTTTTTTTTTCACCTGTTTCCGAATTTAGCTGACAGTG
AGCCAGCTGGGCCCTGAGGGCTCAGGTGGGAGCTGTCAACACCGCTTTGAGCTGGGGTGGCAGAGCAGGG
TGTGGAGGGGAATCCTGGCCCCAGGCACATTTGCCTTTCCGCAGTCAGAGAGGGCTGGGGTTTCTGTTTC
TTTAACCCAGAACAAAAGGAGACAAGACTCAGACAATGGTAGCTGGTGTCTTCTCAGCTCCTCTCCTCCT
CATAGGGCCTGAGTGCACACCACACCGCCACGACTGGGCCAATTTCAGGGTGCCTGTTGTGCAGAGGTTT
CAGGGGGTGGTGGGGGCATCTTTTGGGAGTGCTATGAAACAGTGACACGGGGTCAGGATGGCCCTGGCCT
GTCACTTCCGGTTTCAGGCCCAGGCTGTGAGTGAGCTCTGTCCATCATCAGGAGGACTAGGCTGAATTTT
ATCTCTGTCCAGTGCTGCAGAGCAGTCAGGGACAGTAACGTGAGCTTGAGTTGATGGCAGGCTGCAGGCC
CAGGCTCTTGTAGGTGGCACAGCTTTGGGCAGGCTTTGGCAGATTTTCCATCAAATGATTTCTGCAATGG
TTTCTTTTGCCCTCTCACTCCCCGGCCCTAACAGCAGTCTCTCCACCCACATTCACACTCACTTGGAGAG
GAGAGTGAGGGGGAGGTGTGTCCTTCACAAAGTGAGACAGCAGCAGGACGTGGGTCTGGAACTCACCATC
TATTCAGCTCTCATCCTGTGAGCCAGGCCCAAGCCACACCCAGTGTCCTCATGAGTACTGACTCGGGCAT
GCCACCCGTGGCACAGTGTGGGTGGATGACGTGAGGTGAAGGGTAACCCCATCACCCAGCCCTCAGATCT
TCACCACTCCACACCAAACCAGGTGTCAGCTCCCCTCAGAGAGGATGTGTGCTAGGACCGCCCAGCCCAG
TCACAGTGCAGCCCCTCCCTGAAGCTTGGCAGGTCAGGTATTTATGGCGGGGTGTAGCTCCACGTGGCCA
GGGCTGTTGCTGGAGAGCAGCCTGCTTCCTCACTAAGAACTGGGCTCACAGGATGAACCTGACATAGTTG
GCACCCAGTAGGGGGTCAGCGAATGCTGAAACTTCACTGTTTTCTGCATCTACTTGGAAACCACCAGAGC
GTGCACAAGTCACTCTGTCTGGAACTTGTGCTGAGATGGGTCCCAGAGCCTGTGGGAAGCCAGTGGAGTG
CATGGACCCCAGGGAGACAGACAGGCAGGTACTGGGAGCGGGCCATCAAGGCTCAGCGTTCAGTCTATCT
AGCAGCTGGCAGTAGTGCCGTGGTGTGTGGAAGTGAGCGCCTCACAGCCTGTGGGTATGCGTGAGCCTTG
TCCCACAGCTCAGGAGAGAGTTCTGAGAAAATAAGGTTTTGTGGGAAATATGCATAAATCCTTTGCTCGT
GAGATTGAATGATTGCTGTAATATTTACTTCCTGGACACAGTGCTTCCTTGTGTGTCCTGGTCGGGTCGT
TATTATACCTTCATGGTGCTCAGAATCTAAGCTCAGTACCTGATCGAGCCTTGTTGGCTGGGATTCCAGG
GATGGTCACAAAGCCAGGTGACCTGAGACATCCTGTGCTCTCACTTGACTCTGACCTGCCAGGGGTCCTG
TCACCTCCTATGTTAGATCACACCCCACTGCCCCTGCGTCCTTCTGAATAGTCCATCTCTCACCAGACTT
CAGCCCCACCTCCTGGGTGGCTTCTGTAGCCAGTCTCCTGGAGCAGTGTAAGAGTTCCTTTTAAAAGCTC
GGAACTGGCTGGGCGTGGTGGCAGCACTCGGGAGGCAGAGGCAGGCGGATTTCTGAGTTCGAGGCCAGCC
TAGTCTACAAAGTGAGTTCCAGGACAGCCAGGGCTACACAGAGAAACCCTGTCTCTGTGTATCTCATGCT
GTGGATCAGACCACCCAAAACTCAGCTGCCCAGAGATAGGGAGGGTAGTCACCCGAGTCTCTCGAGTTCA
TAACTGGAGCAGGACACAGCGGGGTAGGTATTAGCTCTGAACATACAGCATTGTGAGTGCTAGAGTCCTC
GGAATGAGAAGGCCCACGCACTCACAGGTGACTTGGGATGGGAGATGTCTCGGTTTCTCGTCACAGGAGC
ACCAGAGTGGTCTCACCCAGGTGGGCTGGCGTGAGAGCCCAGTTCAGGGTGTATAGACAGGAGCACACTC
CTGGCAGGTGATCTGCCCTTTCTCCCCAGTGACACACAGAACCATTTGTGTCCCCAGAGTAGGACCCTCC
AGACGCAGCCCCTGACGGATGCCGGGCTTGCATTGTGAGGGCATATGGAGTGGGGACCGTCCCTGTGGTG
GGAATGTTGAACCACCATCCCTGGGTACATCCACATCCTGCTGAAGCCTCCTTGGGCGCTCGCGATGCCT
TGGATGCTTCAGCTATGGCTGCCACACCCGGCAGCGAAGCCTTCTCATCTGCCCCTTGTAGCTCCACATC
CGGTGCCAAAGGTGAGGTTTTGTTTAAATCGATCCTGCAGCTTCATGGAGCTTCCTGTGTCCCTCATGGG
AACAGTGTTTTTATTTCTTTCTTCACACACTGTCTGCCTCCATTATTCTCCCCCTCTCCCTCTCTCCCCC
TCTCCCTCTCTCCCCCTCTCCCCCTCTCCCTCTCTCCCTCTCTCCCCCTCTCCCTCTCTCCCTCTCTCCC
CCTCTCCCTCTCTCCCTCTCTCCCTCCGAATTTGCTAGCTCTTCCCCGCTGCCTTGGGGTCTTGGTCCCA
CCAGCAGCTTTGCTGACAGACCCCAGGATCTGCTACTTGCCAAGACCTCTCTCCAGGAAGGAGGCAGAGT
GGTACTCACTCCTTCACTGCCTGTTCACTTGGAGACCTTGGGCATGGTTTTTTCTTTTCATTAATTAATT
AATTAATTCACTTTTCATCCTGATCCCCGCTCCTCTCCCTCCTCTCCTCCCAGTCCCACCCTCAGAAGCC
CCCCCACCCCACCCCCGTGGTTACTAACCCACACATCAGACACATCAGTCGCAGCAGGACTAAGCGCATC
GTCTCCCACTGAGGCTCAGCTATTAAAAACAAAGATATGGGGCTGGCAAGATGGCTCAGTGGTTAAAAGC
ACTGACTGCTCTTCCAGAGGTTCTGTGTTCAATTCCCAGCAACCAACCACATGGTGGCTCACAACCATCT
GTAATGGGATCCGATGCCCTCTTCCGGAGTGTCTGAAGACAGCTCCACTGTACTCATATGCATTAAATAA
ATAAATAAATCTTTAAAAGAAATAAAAGAAAGATATGGTGCATCCATATGAAGGCAAATAAATGGAGCTA
GAAAATACCATCCTGAGTGAGGTTACCCCAGACCCAGAAAGACACTCATGGTCTGTACTCATAAGTAGAT
ATTAGCCATAAAGTACAGAGTAACCATACTGTAATCCACAGACCTTAAGCTAAGTAACACGGAGGGCTCA
AGGGAGGATGTTTGAATCTCACTCAGAGGGGGAAATAGGCATCGGAGATGACTAGAGGGAGGGAACCTGA
CCTCGGCTATCTTAATGCATAGCATGGAGATGGGGATTCAGGGGTGGGTGGCCTGCTTGTTTCCAGTAAA
GCCCAGCCCTGCTGTGCTGCACTGTGTCCTGAGCCTTGCCGCCAGCAGTGCCAGTGCAGTAGAGTGACCT
TGAAGGCAGAGCCCGGTGGAAGGCTGGCTCTCTGAAGGGTTCCTGCAGGAATTCAAAGCTGAGGTTCACC
AGCATTTCTTGCCCCTCCTCTCATTGCCAATTCTGGTGTGTGCTGACCACCGCCATCCCTCTCAGCACAC
TGCCCCCCACCCTCGCCATGACCTCTAGTAAGGCCTAATGAGGTCTGTTTCCAGATTCCAGCAACTTCTA
CCCAGGGCCGTTGTCGAGGACAAGAAAGGGACAGGGCAGGACCTTCTGTCCTCCGGGGTGGGGTGAGGTA
GGCCGAGAACTCCCCAAGCATGCCCCACAGGCATGCTGTCCTGTAATGCTGTGGTGGAGGCCAACGCGAG
TGAACAGAGGGACTGCCTTATACTCTGCTCAGGCCTCTGGAGGTTCCAGGAGACAGAATGGAGGGGGCCT
TTTCCTGCAGTTCCTGGTGTCTGCTAGTGTTTTCAGACTTTGTGTCCAGAGCCAGGCAGTCCAGTCCCTT
GCAGACTGTTGAGCCCAGAGACCTCAGAAGCCAGGATCAGGGCAGAGACCCTGCGGTTCCAAACTTGGCC
AGCATGGGGCTGCGGAAACAAATTGATCAGAAATAAAGAGCAGGGATAAATGCTCTGGGAAGCATGAGGA
CCACGGGAAGGATGAGGCGTGCAGGGGTGTCTCCAAGAAAAGAAGCTGGAAAATGTTGAGGGGCTCCCCA
ACACATCCATACTTCTGACCAACTATTTCTAAATCCAGGGGGTCCCCAGTGCTCCTTAGGTTCAAGAACG
GAAACTCAGGAAGCCAGTGGCGGGGGTGAGGGGCAATGGAGAGATGGCTCAGCTGGTCACTTGCCGTGCA
GTCATGAAGACCCGAGTTTGGCTCACCAGCATCCACGTTAGAAGCTGGGTGTGGTGATGCTGCACATCTG
CAACCACAGCCGTGGAGGGGGGAGGGTGGGAGAGACAGGCAGATCCCTGGAGCTAGCCAGCCTCACCATT
CAGTGAGTTCCCACGCAGTATGGCTGTGCCCTCTCCTCACAGAATCACAGCTCTCCCCCTTCAAGGCATA
TCTCACAACCCAGGAGGCTATGCAGGGCCTGGCTGTCTGGAATTCTTTGGGATTTCAAAAACAGGCAGAG
CTGGTTGAGTGTTGTCCATGTGATGGAACTCAGTCCCCAGCCTGCCTTCCTTCCCTGGGGGTTGGACTGA
CTCTGGGCCCCAGCCCTCTGGTCTTAGGATTGACAGCCCCATCCTGAAGCCACCTAGGGAACCACCACCT
CATTAATATAGGCTCAGGTAGAGTGACTAGGGCTTAGGACAAACAGAGATATTCCTGTTGTGCCCCCCCA
CCCCCCACCTCCGCCCTCAATTCCAATCGCAGCATTTTCCGTCCTCACCCCAAGCCGAGGAAGAGTCAGT
TTCTTCATTACACAACCTTAGTGGTGGTGGTTTTGTTTTTCTCCTGTCATTTGTCATTCTGGAAATTACC
TCTGCTATCTTAAGTGACCTGACACCTCTTGCTCTTTAGCATTTCCTGTGTATTGTTCCTCTTATGACCT
TCTCACTAATTAACATTCTCTGATGTCTTTGTTGTGTTATAACGAGGGAGGTCCAGAGGAGTGGTGACCA
CCTCAAGGTGGTCATGACCACCTCAAGGTGACACAGCTAGTAAGCGCCCCAGTAGGATTTTCACCTCAGC
TGGGCTGGCCATGAGGTCTTCTGGCTGCAGCTCAAACCTAGTCTATCGGACAGGTGACTGCCTCTCCTTG
GCCACTGTTGGCTGTCAGGAATGACTACACTTGTCTGTTACGTGCTGTGCACAATAGATTCAAACCCAGC
AGAGAGCTTTTCTATGGATCCCATTGGATTGACCGGCATGCCACTAGTCCCAGCCTGGGGATGGCCTGCT
AGACAGTGGACAGGGCAGAAAGGCCTTAGGGACCTTTGCCTTCCCTGTCACGGGTACTTATCTCCTGCAC
TCGCTTCTGAACCTCTCTGGACTGTGGCTGAGTCCTGGTGGGGCAGAGGAGAGAAGAGCTCCGAGTAGTG
ACCTAACAAATGTCCCTTTATATTATCCTTCCTCCCCTCCCAGATGGCCTGAAGTACAGCAGTTGGCATT
CTTGTCACTGCTGTGTCCCAGGGGACCTGCACGGGGTTGTGTCAAAAGCCAAGCCCTGCAGTTTGGGAAA
TTAGCAGCATTTATGGCTGTCCCAAGAGACTCTTGGCATCTCAGCCAAACTAGCAAGTCTTCAGCTGGGG
ACATTTCTGCCCCAGTAAGGGAGTTTACGGACTGCAGTTGTCACAGTATTTGGGGGGGTCCTGTTGTCAA
TTACGTAGTGGGATCTCGAGGTGCTAATGACCCAGCATTGAGCTGGACAGTCAGACACACTCTGTGGGGA
ATAGGCTTGCCCTTGAGAGACCTGAGAAAGGTCTGCTTCCCTGCCCCTTTCTCACCAGGGTTTAAGTTGA
ATAAATGGGAATCAAAGAAAGTCTCCCTTGAGCCGATGAGTGGTCAGGTGGTCTATCCAGGGGGGCACAG
GCAGCCATGAGAAGAGCATTCTGTTGGGATCTCATGTCAGCTGGGGCCTGGGGTCAGGGCAGACCTGGAC
TTGGGTGGTAGAGGGTGAAGAAAGGGTAGGCCGCCACCTCCTCCGTGGCAGCCTGGGGACTGACGAGTGT
GGCCCCCTAGAGCTCACATCAGCACCTTGGCTTGGGCTCTTTCTCCTGGGGTCTGCTTCTCTGCTTTCTG
TGGTTTCTATGAGCCAGCCTTTCAAGTGCGCGTGTTTAGAAGACCCTAGCCATAGGCTGGTCTTACGGCT
GAACCTCTCTCTCCTTTCTGCCTTGCAGTCGGGAAGACGTCTCTGATCACGAGGTTCATGTATGACAGCT
TCGACAACACCTACCAGGTAAGACTCCCCACTTCCCATGCTCCCCCCCCCCACCTCCCCTGCCTCTGTCT
CTGCCTGTGGACCCTGTCTGGAGAGCACTGCCAGCTGTCTCCAGGGAGCATTGAGCTCAGAGTGGGTGGG
GCCACTGCAGCTCTCTCAGGCAACTTACCGATTGGAAGGAATGCCATGCCCTTCCTGGTAACCTGGCCTA
GGCCGGAAATGAGCTCAGAGTCTAGATGTTGAAAAGGAAACTTTTATTTTTTAAGGGAGAAATATCAAAA
CCAGAGAAGGGATCTTTTGAAGAGGCAGGTTGGAAGCATTACAGAGTTTGGTTATGTCAAGGGGCAGGCC
GAAGAGGATAAGATGAAGTGCAGGGGCAGTTGGAGAGGGTGCAGCTCAGGAGTGGTCACACTGGCTGGAC
TTGACATACACATAACCCAGACAGACCACGCACGGACTGTCACACACGCTCAGTCCGCCCTCATCCCCTG
CTGCTGAAGCACACGCTGCCACAGAGAGGCGTGGACGCTTAGTTGACCCTGCCCTGTGTGGTCAGGAGAC
TGGGCATAAAGACATCTGTGGAGCCTGGTTTGGGAATGGTAGCCTGGGCTCAGTCACAGGCCCATAGGTG
ACCCCAAACCGAATTCATGAGGGGCAGAGACATATCTGTTTGGTATCAGTGGTGGTGGTGGACAGGGCAG
GCAGTACACGTGAGTTTCCTCACAGCTAAAGCATTTTGCCAAAGTTGTTATCCCTACCACCACCTTGGAC
TGTCTGTCCACAGTTAGTTCACCAGTTCATTTCTCTAGGCTAGGGTCCTGTGAGATACTAGCTCCCAGAC
AATGTTAGTAGCTGTTACTTGGCAAAGGGAGTGTGTGGTTTCACAAGCAGAGCTGGGTTGTTTAGACTGG
GGGGGGGTGGTGGGGGGAATGAGGGTGGGAGTGGAAGGGGACTGCTCTCGCTCAGCACTGAATCTCAGGG
CTCCCCCTCAGCCCAGGGCTTCAGTCTGCTGAAAGCAGATGCAGTGGTTGTTTGAATGTAGAATCCCTTT
GTCAACACATTCTCAAGGACAAGTATTTATAGATTCACTGGGGCAGCTGTTCCAAGGTGCCGCTCTCCCC
GCTGCTGGGTGTGGAGCAGCCCACGAGTCTCCGATGCTTGTCCGTCCGTCAGTGCCCTAGTGCAGTCACC
CTTGTCTCAGATGTCCTCTGAGGACCCCCTTTCTGTTTCCCTCAAGTTCTTATTCCACTCTGCAATGGGT
GGACTGAAATCCTATATCACGTGGACTCCAGAGGGATTGCCTACGGGGAAGGACAGGAGACTCTGGGTGC
GTCAGAGGGTGTACGTGGCTTTGGGACATTGATGCCGGTCATACTGTTGTGTCCTCCCCCAAGCCAGGAC
TTTGCGTCACTTTGGTCTGACTGTGGAGCAGAGGTGACATCCCTAACAGACTCCCCTCCTGAAAGAAAGA
TGAAATTTCAAGACCTGTAAGTCATATAAAGTACTCAGAAATTGCTGGTTGTTTGTGAGCCTAGAGGCTG
CCTGGGGCTGAAAATAAAGAAAAACAAACCTGGGTATGCCACATAGTTAAAACATTCCTGGGAACAGCTT
AACCTATAAGATAAAGAAGACTGTGAGGACAAGCAGGGCTATCTTGTCTGGGTCAACACCACCTGGCCGG
AACCTCCCCTCTGTCTACTGCTCCTTGACGCCGGGTAAAGTCATACATCAACTGGTTGCTATGTGAACAA
AGATAAGCCCCCAGCCCACAGAAACAAAGTCCTGATGCCCTTTTGCTGACATGTAATCTTTCTGTTAATG
TTTAAATAAGCCAATAGTGTGTCTCTATGCTGAATTCCACACCCCTAAGCCCCTTACCCCATAAAAACCC
CTAGCTTCTGAGCCTCGTGGCCGACATCCGTTATCTCCTGTGTGGGATGCATGTCGGTCCGGAGCTCCAT
AATTAAACGTCCTCATGTATTTACATCAAGATGGTTGTCTGTTCGTGATTCTTGGGTGCACGCCGAATCG
GGAGTTGAGTGGGGGTTTCCCCACTAGGTTCTAACACTCCTAAAGTCTCCTTGTGCTCTGTGTGTGCACA
TGCACACGTGTGAATGTGTGCACCTGCGCACATGCTTGTGTTTTTATTAGTTGACTTTTCCCTTTTTGAC
ACAGAGACTCCTGTGGCTCCAGCTGGCCTTGACTCTTCTGGGTAGCTAAGAATGACAGTGATCTCTGGCT
ATTTCTGACTACTGCTTGATTTTTTTTTAGCGATCTTCCTGTTTTTCCTATGGCACCGCATTGAGATTCT
TAGCAGCAGTGGACAAATTACCTTTGAAATTTTATCTCCCTTCTTCCCACTGTCACCCTCATTGCCTGAC
GCTTGTCACTCAAATCAGGCTTTACTTGTTCCTGTCACTGTGTGGTGGCTGGCGATGGCCTAGCCTGGAC
ATCAGCTCCTGTCACTGTCCCAGCTCCGCTCGCTCTGCTCTGAAAATTCTTCCAATGCCTGAAGCGTGGC
AAATGTTTGCCCTGAGAACAGAGCCCCACCTCTTGCATCAGTTCCCATGAGCCTTTGGCTGTAGCGTGTG
ATCCTGAAAGCTAGTTGGGCCCCTCAAGTCTAGGGCTACTCCTCTGCCCTTCTGGTTCTCACCTGTGTTT
TGGGATGTGCCAAGTCCAGGATCTTCTTGGAGACATCACTGTACAAGGCCTGGCTGAAGATGGTGCCACA
GGTACCTACGGGCACAGTAATGGCTGCTTGCCTCAGTTGACTGGTGCCATCAGACTGAGCGCCTCCCAGG
ACCACTGTCATGGTGAGTCAACGGAGATCACAGAACAGATCCGATGCCGCTCTCCCCATGCGTGGGAGAA
TTGTCTGACTCGGTTTCTTTACATCACCAGGTGGAAATCCTTTCCTTAAGCCCTTTAGAGCAGTAATTAG
GCTGAGAGGCTGGGCTCTGCTTCTCTCCGTCATTCATACACACACACACACACACACACACACACACAAA
GACATACACACACACATATACACATACAAAGACATATATATAAACACACAAAGACATATACACACAGACA
CACAAAGACATACACACACATATACACACAGACACATATATACACTCACATATACACACAAAGACATAAA
TACACAGATACACAAAGACATACACACACATACATATACACACATAGGTACACACAAAGACATACACAGA
CACACAGACACACACAAACTCACACACAAAGACATACACACAGATACATACACAGACACACACATACAAA
CAAAGACATATGCATAAACACAAAGATACACACACACAGACATATATACACAAAGACACACAGATACACA
AACACACACACACACACACACACACACACAGACATACACACACACAGACACATGATTGCTTGTTAATCCT
CTGGAGATGGAGGAGGACAGACTTTGTTTATTTTCTTTTTCTGAGCATAGCAGCAGAGGTGTATGGGAAA
TAGATAAGTCCCCAGGGACCCGAGAAGATGAAATGTATTTTCTCTCAGGCTGTCCCACTGCACAGGAGCC
GTGCTTGTCACCCTCCCACCAATATCCTGTCTGTCCCAGCAGCAGTACAGCATGGCAGAGTCTAGGCCCA
CTCAGTCAGCTCCCAGGACAGTGTTCCCTCTCTCTCCTCATGGCTCCAGACCCTGTGCTCCCCTCCCATT
CACTCATCCTGACAAAAGACAGCTGTGTGCTGTAGTACATGGCCTGACAGAAGGTAGCAGAAGGCAGCCT
GGCCCACCCTCAGCGGGGCACAGAGGGCAGGGCCTATCCTCAGCCACGGCCAGTGCCACCTTGATGTAGA
GCTGAGTGTGCAAGAGGCCCCAAAGGGCCACCATGCGGCCCTGAGTATGGTAGGCTGGGGTCCTCAGCTG
AACCTCTCAGAAGGTTCTGTTTGTGGCCCTGGTGAGCTGGCACTTCTACAGCCCTGGGGGACCTTTGAGA
AGCATGTCTTCGTTCTCTACTTACCTGGCAGTTACACACATACACACACACACACACACACACACACACA
CCCCTTATTCTCCGCTAACACATGTACTCTAGTCTCTCACCGCCCTGCCTAATCACACCATACAAACTGA
TTCACTTATACAGTTTTCACTTGGTGGAAAACCAAGTCTCAAGCAGAGGAAATTTTGTCTGTGGTGATTC
CTTCATGTAAAACCCCAGCTAAGAGGTAGTATGTTTTGTAATGTGGTAGCATGATGCGGCGGAAGTGGGT
GCCTTAGCGGGCCCATGTTGAGGTACCCCCACCAAGGTACCAGCCAGACAATGGGTCTAGTATAAAATGA
AGTTTATTTGGGGCATGAGAGGAGGCTGTGGAGAGGGGGTGGAGGCAGAGAAAGAGGACAGAGAAGAACA
GAAAGAGAGGAGAGAAAGAAGGGAAGAGGCCAGCAGAGAACATGCAGGGAGAGAGAAAGGAGGAGAGGAC
AGAAGCGGGAAGAGAGGTCACACAGTCTTTTTATAGCCAGCTGGGCTCATACCTGGCTGTTCCTAGGTAA
CTGTTGGGCAGAGCCTAGAAGAAATGCTAACGAGGTTTTCTTAAAACTCTATCTAGGGAAAAAAAAAATC
TGGGCATATCTTGTAATAAGAGGTGGTTCTTGCTGTGCGCTCCCCCACCCATACCAGCACATACCCAGGA
AACCCCTGTGGGCCCTGGGAGGGTCAGCTGTGTGAAGGAGCCCTGCTAAAGGGAGGTGTGGTCCTCTGAA
GGTCACTCCTGGCCGAGGCTCTCCAGGGTGCTCTTTCCAGCCTGCAAAGCTGGCAGCTCAGGCCTTAGCG
TCTTAGAGAACTGAGAGGTGGTAAGAACTTAACACTGAGGACCCATGGCCCCTCCTGTCATCCACACCAA
GTTAGAACAGCACAGATCTAATGGGCTGCCTCAGGGAGAGAGGCGCCCACAGGCCAGGAGGGCTCTTCTC
AGAGGACACTTGTGAGATGGGAAGGTGTTTCAGACTCTGAAGGGGGTTCCTAAGAAGCAAATGCTGGTCT
TTCTGGCAGGCTCAGATCTCCTGAGATCTCTTGCCGGCAGCTGATCCTCGAGCCATGGCAGTCTGTTCTG
CCCTGAGGCTTCCCACACTCAGTCTTGGGGCCGCCGCCACAGCAAATTACCACACACTGAGTAGCTTCAA
AGGAACAGGAGTGTATTCTGTCCTCACCTTCCTGGGGGCCTGAGGGTGGAACACATACAAGAGAGCCCTG
AGCTTTTGGGGGTTGGGGGGAGGGCATTCTCCTTGCTGTTGGCTTCTAGTGGCCTCAGATGCTCTTTGTC
ATAGGGCTTCAAGATTTCCATCTGGCCGCCCTCTTCATGAGATCTGAGTAGATAAGGTGGATGGATAGGT
GTGGATTCTCATCTGGGCCCTTGAAGTGGACGCAGCATGCATCTGTATGCATCTGTATGCATCTGTATGC
ATCTGTATGCATCTGCACTCGGGGAGAGGCTCCTAGTCACGTCACCTCACGTGGTGCCTGAAGCCTTGGA
TTTTTATTTCTGTCTCCTGCCTTGTCCTGATTACGTGCTCTTGCCGTGGCCACAGAGACTTCTCCCTGAC
ACAGTGCGGTAACTGGCCTCTTACCTGACAAGCGATGCTTCCTCTTCAAGGTGAATACATATATGCTTGT
ACACACATACACTCACATGCAGATACATACATGTGCACACACATACACTCTCACACGTGCCTGTGCACAC
ACACGCACACATACATATGCATACACATGGCACACACCAGCACACACACATTTATGCAGTGCGCACACAC
ACACACACACACATATACGCCAGCCTTTCCTGAATACTGAATAAAGCTTCTGCGTGCACTCGTGCCTCTC
TCCGCTGTGGCTGCATCAAATCCTCCTCCTCTCCTATCGGCTCCACTCTTGTCTTCCGGTGGCCTTCCTG
CCCACTCCAGCAGCCATCAGTCGCTGGCTTGTCTGGCCCACACACCCTAGGGACCACGTCACTAACACTG
TTTTTCACAGGCTCAGACACTACTGGAGGGGAGGGTGAATGGGCAAAGACTTGGTTGACCACAGTGATAC
TTCTCATGATGTGAGCACCATGACCACCCAGGGACCAGGTAGCAAGGTGTTTTCTGTATTAGTATTATGA
TGAACAGAGCCAATTACATGTATGCACGTATGTATATGAATATATGTTATTGTACACGTACACACACGAT
TACAAATTGGACAGCCATTCTTCATAGAGAAGAAAGTAACCACGGGACAGATTGTGACCTTGAAAAGCCA
GGTGCCTGCCCTGTGACCTTGGGAGCCAGCATCTCCTCCTCCAGGGCACTAACTGAGCAGCTGTCATTGG
CTGGCAGTGACCCGGCCCTACCTGTTCCCAAGAGGCTGTGAGCTCCTCTCTACAAAGCAACCACTTTCTT
GGCTGAGTCTTTAAGTTTACCAGGCTCATTTGCATGCTAATTTGCATAACTTTACCCAGATACTTCGGTC
TGGTGGTTGGCTGTGCCTGAGAGAACAGCAGAGTCATTGTCAGGGGCTTGTCATGCCCAGCTGCTTTCTG
CCATTTACGGATGCTTCTCTCCAGCGCCGGCCGGCCGGGCTCAAAAAGAAGACGCACGCACATACTTGCC
TTTTCATTCTCTTCCCCTGAAGAAATAAATGCTGGCGTCAATTTATGCTAATTGCCATGCAGATTTCCTC
CGGTAGACTTTATTGCTGTATCTTGTCAGAATGTGGATGCAAATTTATATTTCATAATAAATGCACAGAG
CTAAGAAGATTTCAACAAGTGCCGTAGGGAATATCTTGCTCAGAGATCTTCTTGCTCGGATGTGCTGGTG
GAAGGTCTGTGCAGAGGAATGCAAGTACGCCACCGTCGACACCCCTGTCGATACTGTGTAATGCCACACT
CAAAGGAAGATAACCGTTGGGAGGAAATCTGTGCTTCTTAATATTCTTTGCATGTGTAAAATAAAGTGGC
TGCTATTTTTCATAGTCAGTGAGTGCCGTTTGCCAGCGCAAAAGAATAAATTAGCATTGCCAGGTTCGGG
GCCACGTATAACAGTAGACTACACCTCCGTTTCCTTCAGAGAAGACATCACGCAATACAGGCAGAGAAGG
CACAGCTGTGTGAGGGGATCACGGGGCTTCTAGACTGACAGAGGACTCCCAGGCACATGCCTGTTTGTAT
CTCTTGATATCTCATCCCCTGTGGGCATCTACCATCGGAAATGATTTGTTTCATACCTCATAAGATAAAG
AATAGAAAAGCTACCTTGACTATAACTCCTTGGCACAGTGAAAGAGCGGCGTGCCTCTCCTCGGCTCCAC
CTTCCTGAACCTTTGCTGAAGCCCTGGGAATCCCAGATCCTTGGGATGTCGATGAAGAATAGGACAGCAA
AGGTTCCTGAGGGGCTGGGGGTGGGGGTGGGGGTCTGACAGATTAGGAAAGAAACAGTTCCTGTCTCTCC
ACTGCAGGATTCAACAGAGCCTTTAATGTGCCAGGAGAGTCACTCACTTGCTTGCTGTTTCTGCTCATCC
TGAGCAGAGACTCCGAGGCCATACCGCATAACCAGACAGACACCGGGCAGTCTCCATGGCTTGGCTGTGC
CGCTTTGCCTGGAGAGCCCTGGGTCAGCTGGTTCCTGGTAACAATTGCTCATTTCTGTTGACATAGCATA
TGACAGAATGAAGTGCCTTCACAGAGTCTTAACTCCTTTGGTCCAACAGTCCTTAGAGGGAGCTCTGGGA
TCTTCATTTGAGAGGTGAGGGGGTTGGGGGCTAGGGCTAGGCTGGGGGCTAATTGGACTCCTTAGTTCAG
TATGCACGAGAGCTTGGGTCTTCAGTTATGGGGCACCTGTAAATCCCAGCACAGGGAGGTAGAAGTGTGA
GGATAAGAAGCTCAAAGCCACCCCCAGCAACAAGCTAAAGGCCAACTTGGGAGCTGGGGTACAGTAGATC
CTTTCACAAGGCAGAAAAACAAGATAAATAAATAAGCGCAAAGAGCAACAAAGAAAATCCCACTGGCGCC
CACTGGACACATGACCCACTACTGATGGCATGTTGGCCTCTCTGGGGCCAGCTGTAGCCTGTCCTGAGTG
GCCCTGATATACACAGCCTGAGTGCGCTCCCTTTCCAAAGAACCCGTGAGGGTTCCTCATCACCTGGGCC
GCAGGGCAGCGCTGGGGCTGTGTGTATCAGAAATCCTGGCATTAATCCTGGAATAACACACACACACACA
CACACACACACACACACACACACACACACTCTCATACTCATACCAGTCTCACGCACCCATTCATGCCTTT
GTACAAATACAGTGTAAACTTGAGTGTGTTCCATTCCCACGCACATATAAACACACATCCACCAATGCAT
AACTCTCCTGTGTGACCTGTGTTGACCCCTGAGAGGAGGCTGGTCCAGAGGAGTCACCAAGGATCCCTGG
GCCTAGCCGAATAGTTAGTTGTTCTTGCTATCTGGTGTCTCCTTCCTCCAATGTTCACCTGAAGTGGGAG
TGTCCTTCATGACCAAGACTGCAGGTGCAAAGGAAAAAAAAAAACTAGCTCTGCAAGCGAGGGCACTGGG
CTAAGTGTGGCAGAAATACCGCCTGGGGCTCAACATTTGATCCCAGGTTTCCTGGTGGACAGGAGAAACT
ATATGCTTGGGTCTTGTAAATATAAATACAAGAAGTGTTCAGGTTTGTGTTCTGATCAGATGTTGCTACC
CTGTAAACTAACCCACATCACCCACAGGCTGGTGTGTTTGGATGCTGGGTCACCAGCTGGTAGGGATGTT
TGGGGAAGTTGTGAGATTATATTTAGACGACGAGATGTAGTGGAGGAAGTGTGTCTGGGGAAGGCAGAGC
TGCAGTGGATTCCGTTTATGGCCTGCCTCCAGTCCTGATCCTCTGTGGTGGAAACAGCCTCACGCTCCCA
CCCGCCATGGGCAGGAGCTGCTCTCACCTCCCCTAAAGCCATCAAAATAGAGCCTTCCTCCTGTCAGTTC
TTTCTGTCAGGTATTCTGTCTCAGCAACAGAATAGTCCCCAGTGTCTAGGGTTTCCTGGGGCCAGCCTAC
AGAGATGTACACACATGAAGTGGCTTGGCAGGTAGACTGGCTGGTAGTAGTGTGAACGGCGAGGTGGACT
CCTCACGTGGGCAGTCTGTGCTTCTGACTCACCGGCCTCTAAAATGTGCTGCCCGCCTACCCGCCTCACT
TCCTGCTGGCACCATATTGTACTGTGTAGACAAACACCTGAGACCCGTGGCCTCCACAGTCAGAGCTGAC
ACAGGTCAGAGCCTGTAGGGTGGTACTGAATAACAATTCAACAGTGCTCTCAGACAGAGCCCAGGAGCTC
CACACACTGTGCTCCGGCCATCCCACCTCCTGCCTTGTGGGAATGCCCATCAGTCTGGATCTAGCGTTGT
GGCTCTCCACTTGGGCAGTATGAGTGATGGTCTGGTACTGCCTGCTTTCACTAGTTGGTGTGGTGTGGTT
GCGGCTAACTTTCTGTAGTCTGCTCCAAAAGAGTCAGCCTATATTAGCTACTTTTCCACGAGTATCACCA
AGCACCCAACAGAAACAGCCTAATGTAGAAAAGGTTGATTTGGGCGTGTGATTTCAGAGATGTCAGTCAG
TCATGGTAGAGAACAGAGCAAAGCAGTTTGCATCATGGTGACCAAAAAGCAGAGAGTCGGAGCATGCAAG
CAAGGGTCTGGATAAGGACACACTAGTGACTTACTTCCTCCAACTAGCCCCCATCTCGTATGCCTTACTA
TGTCCCAATAATGCCATTGTCTTAATGAACCCATCAAAGGATTGCGCCATTGATCAGACCAGAGCTCTCA
TGATTTAATTGTCTCTGGAAATGCCATTACACACAAGGGCGGGTAGACAGATAGACAGACAGACAGATGG
AGAAGTACATTATTTATCTCCTAGGTGTTTCTCAGTTTGTTTGTTTGTTTGTTTATCATCCAAAGGCTTA
TGTTTTGAATGCTTATTCCCCAGCCCGTGGCACTATTTTAAGAGACTGTGTGATTTTTAAGAGGTGGGAC
TCTATCTGGTAGAAGTAAGACACTGGGGGTGGGAGGGTGGACCCTTGAAGATTATGCCCAACCCCTTCTT
CCTATGTTGCTCTCCTTTTCCCGGCCTGCTGTGATGTGAACAGCTTTGGCCACATTCTGCTACTGCCAGA
TAGCCTTCCCAAGCAGGTGGGACCAAACATCCATGGACTGAACTCTTTGAACTGTAAGCCAGAAGAAAGT
CATCCCATCCTTGAGTTCTTATTTCCATTGAGTATTTTGGCTGCAGTGATTCAAGAGTCTATACTAATCT
ACTTAATAAACTTAACCACCATACCTGGGCAGTGGTGGTGCACGCCTTTAATCCCAGCACTTGGGAGACA
GAGGCAGGCAGATTTCTATGTTCGAGGCCACAGAGTGAGTTCCAGGACAGCCAAGGCTATACAGAGAAAC
CCTGTCTCAAAATGAAAACCAACCCCCCCCCAAAAAAAAAAAAAACAAAAAAAAGCCAAACTTAGCCATC
ACTCAGCGCACAAAGGCCATCTTTATGTCCTCATCTGAAGCCATGTCCTAGTAGCAGAGAAGGTGTGGGG
TGGTCCTTGTCACCCTGCTGACTCACTATCACACCCTGGGAAAGTCATGTCTCTAGTCTGAACCTCTAGT
TTGTCATCTGCTGAGTGAATATATGTTCACCCATATACCGGCTACTCCACTTGTTAAAACCTGCCTCAGC
CACGCACACAGCCTTTCTACATCTGTCCACATTCGCTAAGTCTGACCTGTCCTGAGTATCATCCACATAC
CCTGCCCCGGAGCCTGCTCCTTCCCCTTGCATATCTTCCAGATATACACAACAGTTCTGTGTATCTTTCT
TACATCTGTCCCATCTACCAGCCAGGATGATCTTTCTAGAGTGCCGGCTATTAGGAAGTCCTTCTGAAGT
TTCACTTCATCCAGTTCCCTGGCTTCCTCAAGTCCCCTACAGCAGCCATTGGCCCCAACTGTTTTCCATT
TTCTGAAAGAACTACCTGGCTTCATTCTTCAGGGTGCTCTCTCCTACAACACTCTCTTTACTGCCTTTAC
CTGTGTACTGACCCTCTCCTGTGCCCGTAGTCCTTGCAGAAGACCCAGGTCCTGTCACCACCATTTCCTT
TCAGGCTAAGGATCGTCTTTAACATAGACTCTTTGTCCCCGTTCATTTAAAAGGTGCTTTTAGTTTATCT
GCACATGAACATTTTTTGCTGGGTTTTAAATTCTAGCTCCACTTGCTATCTGTCATCATTATCTTCCCTG
CTCTTCCATCCTCGGGTACCAGGTGCCATTTTCCTCTGGCTGGTCTCAAGATTGTATCTTTGGTTTGGTC
CTCATTGATTTGACTCTGATTTTTAAAAGCTTTTCTGGGCTGGAGAGATGGCTCAGTGGTTAAGAGCACT
GACTGCTCTCCCAAAGGTCCTGAGTTCAAATCCCAGCAACCACATGGTGGCTCACAACCATCCATAATGA
GATCCAACTCCCTCTTCTGGAGTGTCTGAAGACAGCTATAGTGTACTTACATATAATAAATAAATAAAAT
ATTTTTTTAAAAAAAGCTTTTCTGTACTTCTTGTCAATTGTGGTGGCTTCATGTTCTGTCCTTCACGTTG
TGTTCTAGTTGGTATGATATTATATACTTGTGTATGCCTATGTGTGTACTGTACATGTTCTTGTGTGTGT
GAATGTGTGTGTGTGTGTATGTGTGTGAAGAGGTACATTTCTCATGGCACGTGTGTGGACATGAGAAGAC
AACTTGTGGAATTAGTTTTCCCATTTCTACCATGAGGGTTCCTGGGCCAAACTGAGGTGGTCAGGCTTGG
CAGCAAGCACCTTTACCTGCTAAGCCGTCTTGCTGAACTTGCCCATCTCTGTCTTTGTTATGAACATAAC
CTTCTGTAGTTGGGCATAATAAATATTGGTTATCTTAAAATATTTCTTCCTACTTTCAATGTCCGAATCA
TCTCAGGATTGATCCTCATTTTCCTTGTGTATGGCTTGCGTTTTCCTGTCACCTCATGTGTCTACTCGGT
TGAGATTACATCCTGCGCGCGCGCGCGTGTGTGTGTGTGTGTGTGTGTGTGTGTGTGTGTGTGTGTGTGT
GTGTCTGTCTGGATTCTGGTTGCAGCTTTGAGTCCTGCTGGTTCTTTGTTTTAAACCCTGATTCTCAGCT
CTGTCCCCTGTGCTGGGCAACAACCCTCTCTTCCATCTGTAGTCTTGTTTTGCTTCTGGCCTTGGGCAGA
TTGCTTGGGATCTTCTCTGGCCATCCAGTATTGGACAGCATTTCTATAGAGACTTCAGAGCCCCTTCTGT
GGCTTTCTCATGTCTAGGATTTTTCTGTACTTCTCGTCAATTGTGGTGGCTTCACGTTCTGTCCTCTCGT
GTTCCAGTTGGTATGACTGACTGGGTTTCTTCATGAGTCTGAGTTTTTACCAGCACTTCCTGGTGCCTTC
CTCGCCTGTTTTTTCTAGTTGATTCAGAGTTAATAGCATCAATCATCCAGAGGGCTAGATGACCTCGGAT
GATGAGAACCAGAGTCCACCTCACTCCTCCAAGATGCTTCTCCTATCCCCACTATTCCCCGACTTCTGCC
TACTGGAAGGTCCTATGTACTGAGGTCTTGCGTATCCACTCCACATATCCATCAGACACCGAGCAAGCCA
GGCCTGCTGCTCTGACCTTCATCCCTTCCACGCTGGCTCTTCCTCTCCATTTGCTGTGAGTGACAGTTTC
TGTATGTGTCTTCTCCTCCAAGTAGGAAGATCCTTAGGGGCAGGGACTGGGGATTTTTTTTTTTACAACA
CACAGTAGGTACTCAAGGGTGAAGTGAAGTGGTTTGACTTAATTATTTACACCATCTGCCACGAAGAAGC
TGCCCAGGGAAGACCATGACTGCCACCTTCTCTGAAAATCTGGCCTCCGTGGGAGACACAGGCTCCAGCT
GGCAGCGGTGTGGGCCAGCTGCTGAGACTCATGTCTGGGAAATACATGGTGCCCAGGAGCTAGAGCTATG
TTCTGGTCATGGAGACCCAGCCAAGTCTCTCTTTCAAGCCCTTTTCCCTATGTCCTTTGAGGGCTCACTT
TATTTATTTCTACTAAAATGCAAATGAACTCTCTCTTAAGAGGGCTCTGCAGAGCTGTTATGGCCAATGA
TTACCGCTTTCTGACAGTCGGGAGCCAGACAGGATTTGACTCCAGGGAAACACAGTGACAGCTTGGAAAA
TGGACTTCTAGAAGCCGCAGGACTACAGCTCAACAGCCCAAAGCAGAGCCTTTGACGTCCGGGAGAGCTG
GATTCAGATCTCAGGTCTGCTACCCAGCCTGGGTGACAGACCTTGGTTCGGAGTGAGCCTGGTTCCTTCC
CTGTGCATAGCAACTCATCCACTGGTGTTATCTCCATCTCCCCCACCCTCTTGTTATCAGTCTGTGGCCC
TGGAGAGCACAGCCTCCTCAGAGCTGGATCACAGCACAGAGCAGTTTATCTGCACGCTTCGCTGAGCAAG
TCACTGCTCTGAGAACTTGTCTGTGATTCTGACACGCAGAGCCTGTGCTTGTGTCTACCTGAGTGGGCCC
AGGAGTCGCAGTGTTTAAGGAAGCTCCTGGGGTGACACTGAAGCACATGATCCACATGGTACATGCTAAA
ACCCCCCACATGGTTGACCAGAGGCAGAGATTAGGGAGTCTGAGACATTTCCTATTTGGTAGAGAAGGTA
TTGAATAGCCTCTGGGCTATCCCAAGTGTTGATAATCTGATTTGCGGAGAAGCATCTCAAATTCATAAGT
ACTGATTATGGAAGAACATTTCACAGGAGGGACAGCATCAGGAAAGAGATGCAGAAGCTGACATGGGAAG
GGCCTGTAGACTGACTCAGTGGGTAAAGGTGCTTGCTGCCAAGCCCGGTGACCTGAATTTGGTCTCCAGA
TACATGTGGTGGAGGACCAGCTCCAGCAAACTGTCCCCTGACTTCCACACAAAAGCCATGCATGCATGCA
TGCACACACACATACAAAGTGAACAAAAACATGCAGAGAAACATGTGTCTTAGTTAGGGCTTTATTGCTG
TGAACAGACACCATGATCAAGGACAACATTTCATTGGGGCTGGCTCACAGGTTCAGAGGTTCAGTCCATT
ATCATCAAGGCAGGAGCATGGCAGCATCGTGGTGCAGGAGGAGCTGAGAGTTCATCTTCATCTGAAGGCT
GCTAGAAGACTGACTTCCAGACAGCTAGGACAATGGTATTAAAGACCACGCCCACAGTGACATACTTTTT
CCAACAAGGCCACACCTCCTAATAGTGCCACTCTCTGGCCCAAGCCTGTACATGACATGTGAATTCTAAA
CAGCATTATTCCTTGGTATAAAGTCAAGCTGGTTATGAGGAGTACAAGGTTATAAAAAGCCAGTGGTGAC
TCTTGAACAGTCAACCACAGGTGCTTAGACTGTTTCCTGGGAGCTGTAAGGAGGAACAAAGTCAGGATGT
GTGTGTGTGTGTGTGTGTGTGTGTGTGTGTGTGTGTGTAATATTTATATTGTATATAAATATATAATACA
TATTATATATATTATTAGTTTTGTTTCTTAAGATTCATTTATCTTTATTTCATGGTTGAGTGGTTTGCCC
CCATGTAAGAAAATGCACCATATATGTGAAGTGCTGGTGGAGACCAGAAGAGGCCATTAGATTCCCCAGA
ACTGCTGCAGACCCTCTGGGCCCTTTGTCTGCGTGGAACGGGTCTCTCTAGTCGCGGGTAGGCAAAGCAT
CGAATGAATTGACAGACAGATGCACACAGGAGAGGTTGTGTAGAATCTGAATGTAATGTCAAATCCAGCA
TCAGACTTTTTATACAGAAGACAATAAGGAAGTTGGGTGACATTCCTGCACGGTAAAAAGAGGTTACTGG
ATTCTTACATAAAACAGAGGAATGCAAACACAGAAGGTCTAACAGGAACCACCTGGGATAGAATTCATTG
TTAACAACTGGGATCAACAAGGGCTCCACCTAAGATTCACTTAATCTTAGAAGCCCAGGGTCAAGGGCTT
CGTGCCCTTGCCATAGTTCCTATTTTAGTCTATTGTATAGTCCACCTTCCCCTTAGGCCATTGTAAATAC
GTGTGTGTGTGTGTGTGTGTGTGTGTGTGTGTGTGTGTGTGTGTGTGTGTGTGTGTGTGTGTGTGTGTGT
GTGTGTGTGTGTATGGGTGTAACTCAGCTATAGTCTTAGAAGCCAGGTGTGAGGTCTTTACTCTTCTAGG
GCAAGGGCTTTCACACCCAAGTCATGGTTCTAACTAGGGAGTTCCTTTCTAGCTAACCATCTCATGAATA
ATGCAATACTCTAAAACCATAGCCCGATCTACTTCCTAAACCATTGTAAATTCCTGTATATGGGTGCGAC
TTGGCTTTTATTCTAAGTGATAGTGTAATACCAGAGGCTATTTCTGGATGTCACTGAATAGCAATATTCT
TACTGAACTTCTAGCCCATGGTCTTGCTCAAGGACGTTCTAAGACTGTTGGAACACTGGCGGAAGGCTTT
AGCTATGTCAGAATTCAATCTTAAAAAGCACTTATAATAGGATAATACTAAAAGAGAGCACGTGGATCCG
TACACTAGACTAACTCGGGAATGGAAAATTAATGTATGGGTTAGAGAGAACGCCAGACTCCAGGAGTGTT
TCCGTGAAACTCTTCCGCCTCATGAGTAGCTTCCAAACCTCTCGGCTTGTCAAACTGACTCAACCGGAGT
GCGTGGCATAGAACAAGCTCTATGGAGAGTTGTAAGCTGCCCTGTGGGTGCTAGGAACTTAACCTGGGTC
CTTCCCAAGAGCAGCCAGTGTAATTAACTGCAGAGCCACCTCTCCATATCCCTAATGTCATTGGTTTTAA
ACTCAAAACTAACCTTGCCTGCCACATGAAGGTTGCTTATGGGGCAGTGGTGAGGACAGGGAACCCAGGG
AAGATGCTACTGAGGTTACTCCCGTGGGAGGTTCAGTCTGCACGAGGGAGCAGTGAGATCGGTGAGAAGG
GGTGAGGATGGGAGCAGGTGAAACCACAAGAGGTGGCACCGTCATCTCTGCCTTCCCTCACCCCTGTCTT
GATGGTTTCAGAATGTCCTCAGAGTTCTTGAGACTCATTCCCACATCTATTTTCTTCTTTTAAAGCCTAG
CTATTTGTGTCATCTAGGGGTCTGTTCCTCTTGAGGTTTTTTTTTTTTTCCTCAATTATGATTCAAATTT
CTTCTTTGCATAGGTGGTCTGTTTTTATTTTGTGGACAGGATAATTTAAATGCATAGCTTTCTTTGCTTC
CCCAGAGACTTTAATCCCTTCATTGTTCACCAGCTTGTGGTCTCTCTTACGTGCTTGTACATTGTTGTTT
CTTGAATTTTTTTTCTAAAAATCAGAATAGCCTGATCCAATCTGCTCAGCTCTCAACAAAAGAAAAACAG
TGACATCAGAGCTTAAGAGCAAAGATGGCTCAGACACCCAGAGGTGATGCCCATGCTGTGCTGTACAGAT
GTAAGAAGATACACATTAGTTGAATGAATTACATTTTTTATGTTGTTGTGATTAAGTTACCTGACAGAAA
CAGTTTATGGAAAGAAAGAGTTTTGGTTTTTTTTTGGGGGGGGGGGTTGTTTTTTTTGTTTTTGTTTTGT
TTTGTTTTTTGTTTTTTTTTTTGGCTCATGGTTTCAGAAGAGTTCAGTCCTCATGGCAGGGAAGACACAG
CAGGGTTCACAGCAGTGCCAGCTTGTGGTGGGAGCTCCTCAGACCACAGTAGACCCTGAAGCAGAGAGAG
CAAGAACCAGCTATAACCTGGGGTCCGTGTCAGACACCCACCCTAAATGTCCTGTCTCCACCAGCTAGGT
CCTGCTTCCCAAAGGTTCCAAACTCTCTCCAAAGAACACCACCAGCTGCAGAACAAACACTCAAAACCTA
AACCTGTGAGAGACTTCCAGTCAAGCCATAACATTCCATCTTGGTTGCCAAAGGCTCACGGCCATCTTAA
AAGACAAAGACACACTTATTCCAATTTTAAGACGTTCTCACAGTCTCAACATTGACCAATAGTCTAAGTC
CAAAGTCTCCACTGATGGTCAAAACAGTCTGTGGTCTCCTGGAAATAAGAATGTGTTCAAGATTAGAAAA
AAAATAGGAGACCAAAAGCCAACAGGGGAAACACTTAAGTCTTCTACCTCCGTGTCCAGAATCTGGGCAC
ATCATTTGGTCTCACACAGAGATGTTCCTTAAAAATCTAGGTGCTAGTCTCTGTGACCCCATAATTTACA
TTCTGCACACCTGAAAACTAGCACCAGATAGGTAAGTCAAAAGTCAGCTGCCGCAGGGACCCCTTCGATT
ATGGCTGCTTTTGGCTTCTGAGTACCTGAGAAGCTGAACCAAAACCTAGAAAACCATCTTCTTAGGTGGC
CCCTTCCCATCGGAGCACCTTGGTGACTCTTCAAAATACATTCACCTTCTGAAGGAAAGTATTTTAAGTG
ATTAATTGATTCGTCTTCTATTTCATACCCTGGAGCCCTCAGAATGGGGACAGATTCTCAGCTCACCTGC
TCTGTTGCCCCCGTGCCAGGTGAGCATCAGTAGCATGCCGTGACCCCTAGTCTCTTATCCACAGCTTTAA
ACATCTTCTCCTTTCTTTCAGGGCCAAATTACACATCTTGAAACTTCTCTGCATTTTGCCCCCAGTCTCA
CTGTGAAGGTAGCTAAAAGTAACCAGCCAGAACCATACTGGTAGCAGGTTGACCTCTGTCTGGGTTGCTT
TGCATTTTCTCCATTACCTTTGAAGTAGTCCACCACCTCACTCAAAATTTCAGGTCATGGGTAGAATGTA
AATAAATTCTTTGTCAGATTCCAGTGGCCTCTAGAAGCCAGTGACCAGTGGAGTCCTTGTTTTGCCCAGA
GATCACGTGGGTATGGTCTTTGCTGCTCATATTGTTGTTAGTGTTTGATCCTCCAAGTTCTCAAGGGAAC
AGTGGATCTGGGGCCTTAGAGTCCACATGGTTAAACTGTTGCAAGCCAGTTTCAAAGGCTTAGAAACCAC
CTGGTAAGATTAGCCATCTGAATGTTGCCACTTTGCTGGGATCCGCTCTCTGCTTCTCACTTTAGCATGG
TTCTGAGAGAAACAACTTGAAGAAAACACTCTTTCTGGCTCGTGGTTTTTCAAAGGGCTTCAGTCTATCC
AGCAGTAGCAGCATGAAGTAGAGGCACCTCACGCCACTGAGGCAGATCAGGAAAGAGCGAGGACAGAGGC
TTTGTGTAACCTTCAAAGCCTGCCCCCACCCAACTGCTTCTACAAGGTATGTCCCATTCTGAAAGGTTCC
ACCGCTCTCCAAGTTAACACTGTCAGCTGGAGAACAAGCATTCAAAACACGAACCTATGGGATGTTTCAG
ATTGGAACGGCCAGAATGAGTCTGAGAGAGTGACAGCTATGCGATTAGGGATGGCTCTTAGGTAGTAGCT
TGAAGATCAGATAGAGGTGGGAGGCAGCTGTGGGGAGAGGAAGTTGGTCCCTCTCGGCGTTGGCTCATTG
CTGTCTCCTTGGTCAGTCTGTTTCGTGAGGCTTAATAGAGATATAGTCTGTGGCTCAGTGAGCTTTAACG
ATGATAAAAGTCACTGGGCTACGTGTTGCTGCGGCTTGATCCAGTGGCATGACTCCTCGTCATATTATCT
AATGATCTCTCTCTGCGCCATGGCAGCGAAGTGTCTTACATGTGCACACAATGGGGGAAAAGGATTGTCG
CCGTTACAGTAACATCCCTGTCGCCACCCAGCTGCCGACCTTAGGGGATAGGATTTTCCATGGTCCTTCA
CTCTGTTGATCCAGGTTAGTTACGCCGTACTGACGGCGGCTTGAACAAAGTAGAAAATGAGATCTCTGTT
ACTTGTAGTACTGCCGAGGTGACTCTCTTCACAGTCCAGGAGTCAGGCTCCGTTGAAGTCTTCCTGCCCT
CTGGTCCTCAGCTGGACGAGCCACGATCACCCGCCCCACGAGTCTGCATTCCACGCAGTGGGAAAGGGGG
CAGCTTAGTGGGGCGCACTAACCCAGGAAGGGCTCATTCCGGATGATCATCACCTACACATCCTGCGATC
AGAACTCAGCCACCCTGCAGCCAAGGCTTCTGGGAAACATGGCCTTTGACTGGGGCAGCTCAGTTGTGCA
TTTTCTAAATTCTGTATTGAGGGATGCTGGCAGCCTCTGCCTCTGATCCTGAGCCCAGGCATCTGGTCTT
GCTTCAGGGGTCACTAAAGAAGGAGTCTGGTGTCTTCAAGGATCCTGTGGCACATAGAAAAGTTGACATT
TCTGCCTGCTTGGCATATTCCTGGTCTTCGAGCGCGCATCAGTTTTAGACTTAGTTCTCATGGCTGTGGT
AGGCACTTGGGGAAGTCAGGCAGGAAGCTGGAAGCAGGAGTTGAAGCAGAGGCCATGGAGGAACACTGCT
TACAGGCTTGCTCCTCAAGGCTTGCTCAGCCTGCTTGCTTGTAGCATCTAGAACCACATGCTCAGGGCCT
TCCTGCATCAGCCATGTATCAGGAAAATGCCTCCCACAGACGTGTCCACAGGCTAGTCTAGTCAGGTGGA
GCCAGCTGAGAGCCCCTCTTTCTGCAAACCTTTCTGTATTACAAGAAGATGATTCTAGGCACAGGGTGGA
GCACGCGACTGGAGCCATGCCCTGGATGTCTAGCTGTGTCTGAGGGTTCGTTATCACTTAGTGGTGTTTG
CTCTGTTTCACACCGACAGTATCTTGGCTCCTGCTTGTAGACATGGGGTGCAGGTAAGGACCAGAGAGGT
TGTCTACTAGGACAGAAGAGGTGCCAGGTGGCACAGGAGTGTGGGTGACATCCCTTGGCTTGGCAGCATG
AGGAGCAAAAGAGCTCTTTGGGCTTCAGGCATTGTGGATGGTCCACAGTACCCCATCCAAGGCAGGGCAC
GATGCTCTCTGTCCCCATGCAGTGACAAATCCTACCCAACCCTGCCTCCCCGAGAACAATGCCGAACTGA
AGTCCAAGTCCAAGGGAGAGTCTAGGAGCTCAGGGGATGCTGGAGGCAGGTGGGGACTGCTTCCTGTGCC
CCATGCTCTCTCACCCTTCTAGAGGCACCGTTCTCTCTCTCTGCGTAGAACATGCTCTGCAAACAAAAAC
CTGATCTCACCATCATTTGCTCAATCTCCTTCCTCAGTGGAGGGACTTGGCTTGAGTGTCCCCTCTGTTG
TCTGTGCCATTTCCTGGGGTCCCTGGCCTTCTGGCTCCTTTGTGTGATTCCTTTTTCATGTTGACCTTTT
GGTGTTGGTTTCTTTTTTTTAAACAGGCAACCATCGGGATTGACTTCTTGTCAAAAACCATGTACTTGGA
AGACCGTACGGTGAGTTCCTGGTCTTAGAACTGGAGGACAAGGAGGGCATTGTCGGGCTTGTTCCCTAGA
GCCCTCACTTGAGCACTCTTGGGAGTCTCTGCCAAGACCAGGAGGGAGCTAGCAGTGCCATCATCGTGGG
GTCTGGGCTTCCAACCCCCTCTACCACTCCTCTACCGATGCCTCTGTTTGGGCCTTGTAGGTTCGACTGC
AGCTCTGGGACACTGCTGGGCAAGAGAGGTTCCGCAGCCTGATCCCCAGCTACATCCGAGACTCCACGGT
GGCTGTGGTGGTATATGACATTACTAGTAAGTTGGAGTCTGTGTTGGAAGGAGGGGGCTCTTAGGACCCA
GTGTGACCCTGACCAGACCTGTGTGTGTGTGAATGTGTACGCCAGCGTGTGTTCAGTGTGGGGTAGTGTG
TTATCTTAAACTGTAGTGGATTCATCAGTGTCCTTCAAACTCCTGTGACAGATTCGACTGCATAATACAG
GGTTGATCCTGCTTGGGGCACGGGCCCCCCGGGGTGGGGGAAATGGCAGGTGTTCACAGGGTTAGCTCTC
TGTGTCCTCCCTGGCCCTGGCTGGCATGTGTGTGCCAGCTTTCCCCAGGCATCTCTCGAGGCAGTCCCTC
TTGTATACACGGTTTTTGAAAGGGCTGCACAGACTCAGTGGGGGACCAGACATTGGGGCAGGCCTTATGC
AGAGCCCAGGTACCCCCAAATCATCCAGAGCAAAGATTCTCAACCTGTGGGCTGACACTCTTCTAGGATC
AAATGACTCTTTCACGGGGATGGCATACCAGATATTCTCCGTATCAGATACTTACATTGCAGTTCATAAC
AGTAGCAGAGTTACAGTTTATGAATTTGTTACTGTAGCAACAAAAATAGATTTATGGTTGGGGCTCACCA
CAACATGGGCAACTGTGTTAAAGAGTCATTGCATTGGGAAAGATGAGGACCACTGAGCTAGAGATGAGCA
ACTGTGTTAAAGAGTCATAGCACTGGGAAGATTGAGAACCACTGACCTAGAGGAACACACCTGGGAAGCC
CCAGGCTTTACTCCCAGAGTGCTCTGAGGACCCTGACATCACATGCGGTTGTGCACGCCTCACTGCCCAG
TTCTGCCTTGCCCCTCTGTCTACCTCCTCCTCTCTGAGCATATGCTCCTTACACCCCCTAGTGAGGTGGG
AGTTGTGTCACAGGCTGGGGAGAGCAGCCACCTCCCCCCCACCCCCAGGTGCTGAATTTGGATGTCTGAG
GGAGATGACCCTGAACACAGGTGACTTGCTTCTGTGTTGATATGCCTCTAGGGGGCGCTTTATCTGCCTA
CCCTTCCCTGCAGGGTTGCAGGTTCTCTTCCCCAGCTAGCCTGGCCTTCCTCAGGCTCCTCTGAACATCA
CTTTGGGGGGACACCATTTCCTAGGGGCCGCCATATGCTCCCTTCACAGGTCCGCGGCTCGGCCAGGCCA
TGGCCAGCTGTGACTCTTGCGTGGTCTCTAGAGTCACTGTTCTGTGTAGAAATTGTGACAGCCTAGCTTC
CTAGGTACGGTGACTTCTCTCCCTAGGGTGGCTGAGCAGTGCTGGAGGTTCTCCCAGTATCACAGGGTAG
GAGGCCATCTTTCCTTCTGACCCCTCCGGGGACCTCATTCCCACTCTGTTCCCACTTTCTGCCTCCAGAT
CTCAACTCCTTCCAGCAGACTTCTAAATGGATTGATGACGTCAGGACGGAGAGGGGCAGTGACGTGATCA
TCATGCTTGTGGGTAACAAGACGGATCTGGCTGATAAGAGGTAGGCAAGCACTGGAGTGTGGAGGTGCTA
TGACTAACGGGCCCATGCTTCCTCGCCCCTCTGTGGCTGTTCCTGCCCAGGTCTGAGATGACAGGGCAGC
GAGGCTGATCTCCACGCACAGGGCCCCCGACACGCCTGTTAGCCCCCATACACTGCAGGCTTCTCATCTC
TGATTTTCCTCAGCTCAGCAAAGGCAGGGCAGGGCCTGTCTGCACAGGCATTTCTATCATGGTCCCAAAG
TAGCCTGTGACACAGCTTCTGTCCCTGGGAAATCCTGGCGTTGACATGTGAGGTCCTCAGGTATGAGCCT
GAATTATGTCAGCCGAGTACCGACTTTGTCCCCTGCCCATGCATCCTGGTTTATGGCTGGATCTGGGCAT
GGAGTATTTTTGGAAAAGGTTTCTCCGTAGCTGTCCCCTGATGTCACTGCCCATTAAGGTGTCATTAGCA
TATATGTCCGCTCTTGGAACCCTGTGCTAACTGGGACAGAAAAGCCGTGTGTCGCGGTTTCTAATAAAGC
ATGTGGCTTCTTGCAGCAGCCCTGCCGAGGGGGGAGGGAAATGGTCTCCTCTGCAGAGCAGTATGGGCCA
AGGCGAGCTACTTTTAATTTTGGTGTTTGCATTAGGAAATGGGGGCTGAGGCTTCACTCCAAGAAAGGCT
GCATGTGTCCAGGAGATCCTGTTCTAACGACTGCTGAGAAAGCCGTAGGGGGCGTGGCTCAGTCGCAGAG
CGCTCGCCTTTCCAAGTGTGAAGCTCAGCATCATAAAGCCAGGCATGGTGGCATATCCTACAGTCCCTGC
TCTAGGGAGGGACATATCATACTGAATCAGTAGTTCAGTGTCATTCGTTGTTACATCACAAATCCAAGGC
CAGCCTGGGCTACATAGGTAAAAGGTAGAAGGTAGCCTTGCTTCCTAGTTCAGAAGATGTGGGAAAATGT
GGAAAAGACTTGGGGTTCTCTGTGCCCAGGTGCCACATTCTTGCTAGACAGACTATAGCAGGCCTCCTGG
CAGGGACCCCTGGGCAGGAAACCTGTCTTCACGGCTGACCAATGGGCCTGGCAGTCCCTACAGTTCTTTA
TCGCAGGCTGCCTCCTCTACTTCTGCCAGGGGTTGCCTCTCTGCCCCCTGATTTTCCTTCTCCCCTTCAT
TTCCAGGCAGATAACCATCGAGGAAGGAGAACAGCGGGCCAAAGAACTGAGCGTCATGTTCATCGAGACC
AGCGCGAAGACTGGCTACAACGTGAAGCAGGTTAGGATACAGCTGGCTTGCTCGGGACTGGAGGCCCTCC
CCTGAGGTCTTCCTTGCTCTGAGTGGCTAGCGACTAGGATGGGGCAGACAGCCAGTGCATATACTTATAG
CTCTGGGCAGTGGGGGATGGCACTGGCTTCCAGCGCTGGTAAAGCTGGTGGCGTGGGGGGTGGGGGAGGC
ACTCCGCTAGGGAGCCGCGCAGACCCTGTCAGTCACTAGTCTGTCTCCTCAGCCATCATCAGGTGTGGGT
CCCTGGAGCAGCGTGTGTGAATGAAAGTCCAGCATGGACTTCCACCTGGCTGAGGAAGCTGTGGTTCTTA
GCATCTCATTTCACTTCACATGCAAATGTAGGATGTTGAATCTGATCGACACGTTGGGCTTATGTCTTGT
ACTTGGCAAGAACCCGGGGCCATGAAGGGTAACTCAGAAATGGAGCAAAGAGACTCCCTTGGCAGTCGGG
AATCATGGGAGGGAGCCAAGGTGTGGCTGGGACCCACAGGACCAGAGTAGTTGGTGGGCGGAGCTTAGCA
GCTCAGCCGACCTGGGCTTCAGAAAAATCCCACAGGTATTGTGACTGCTCCCTCATCTGTAACCAGCTCT
TCAGTGATGAATACTGACAGCCAGACAGCCTCAGTGTTAGCAGTGAGTCCTGTGACTGTCCCACTGGCAG
AACTTCACAGTATGACAGGGATCCCACACTTCACAGTACTGGATCTCAGCATCCCACTAGCCGTTAGACC
ACCTGTAATCCTGATGTGAACATGTATGTTTGTAAGGAGACACGGATGTCAGCGTGGCTCAGATTTATGA
TCCTTAAATATTTGGGAAGTTTTATTCAGAAGCAAACGGTTTCCTTTATCATCCATTATAACTTGACTAC
ATCTGAAAAAAACGAGTCCACCGGCTTCAGCAGTGTGAAAGGAACCCATGGCTCAAACAAGGTGAAGAAC
CCCTGTCTCGGAGCAGGAGGAGGGTGGTAGCAAGGAGGAAGGACATCCTAGGCAGGGCTGATGGGCGTAG
AGCCTGGCAGCAAGGTGCCCGTAGCACGGGGAGGGACCCTGAGTAAGGAACGTGGCTTGATGCCGCCGTG
TCTGCTGCGGCCCTCGACTGACTTCCCAGGCATACCTGGGCACCACTTGGCTTTTGAGAGAGAGTGATGG
TTCACTTCATATTGATAGTGCTGAGATTAAGGTTGACAAAGGAGCTAGAAAGGACAAAGTAGGAGGGGCA
CTGTGGCCATATTAGGGAACCCCCGGCCCCAGCTTCCGGAACCCAGTGCCCAATTGGAGGGCCCTTCAGC
TCCATTCACTCCAGCCCTTTCCAAGCCTCTGTATCAGATGTGAGCCTAACCACCTCAGAGAGGAGAAAAA
GAGGCGGGGGTGGGGTGGGTGGTGAGGCCGCTGCTCTTTGGGTCCAATGACATCATGGGAAATCGAAGCC
GAGGACAGTATCAGGCATCCATAGAGCAGGAGGCATCTTCAACCTGCTGGGCCAAGGTGAGACTGGTCCC
TTCCCCCATCCTGGAATGATCTGGAATCTGAGCAAGGGTTTGAGCAAGAGTTAAGTCTGGTGGGTTTGAG
CTGCTATTGCAATTGCTACAGGCTATGCAGCTCACAGGTAACGGAACCTTCTTCCTCCCTGCTCTGAAGG
CTGAGGACTCCAAGATGAATGCGCTGGGAGATCCAGTGCCTTGCGGACTCTGCTCCCTCATATTTGGCAC
CTTCCAGTTGTTTCCTCACATGAAAGGAACGTGAAGGCTCCCTCAGCTTCTCTTCCAGTCAGGAGGGCTC
CTTAAGGCAACACCTCCTCACACCGCCTTGCTAGGAGTTAGATTTTTTTTTTTTCATTTGTTTAGTTGGT
TTTGTTGTTCGTTTGGTTGGTTTGCTTTTCAGTTTTTCGAGACAGGGTTTCTCTGCATAGCCCTGGCTGT
CCTGGAACTTACTCTGTAGACCAGGCTGGCCACAAGCTCACAGAGTGCTGGTGTTAGAGACATACACCAC
CACTGCCTGGCCAGGAGTTAGGTTTTAACTTATGAACTTGGGGGAGACCCAGACATCCAGCCAGGCTTAG
AATCTTCTGTGCTCTTGGCTTCTGCTTGTACTTGTTACTTTCAAGATGTCACCTCTGCCACTGTCCATCT
ATTAGGTGCCCCTGATACCTTCTGTCCTCGTGCACACTCAATTAGGTAGACTTGCACGTCGACCCACACT
CCACATGCTTGCTTCTCCCACACCCACTTTCTCATATACACGCAGAAACATGGCATTTACCATGATACAC
ACACATACATACACACTTACACCCAAAAACACACTTAACTTTTTATACCCACTCTACAAACAATATCAGG
CATACCACGCATAGCACACGCATGTAGCACACCACATTGACACAAATTTTCACATCCTTCACTGTGGCTA
TGGCCCTGGCTCCCAGGATCTGTGTCCCTGGGGAGAGTCGTTTGTCTTACGTAAGGCTAGAAACTGGTTT
TCTACACCTAGTCCCCATGTGAGAGTGATGGACAGACACCCACTGAAGGGCCTGCTTCCTGTGGCCTGGA
TGATTTCTGTAGCAGAGCAGGTCCCTTGCAGTCACCCTCATGACCCCTGCTGACTGCCTGTGGGCCAGCC
TGCCTCTAGGAAGAGCCCCAGCTTTGCTGTATGTGTGGACATGTGTTTGTGTGCAGGTGTGTGTGGGCAT
GCATGTGTTTACTTACACCTGTGGCGGTCACAGGTTAACTTCTGGTGTCATTCCTCAGGTGTCATCCACC
TTATACTTTGAGATAGGGTCTTCGACTTTACCTGAAGTGTACCAGTTCAGCTAGCCAGGGAGCCCTGGGA
ACCTGTCTTTGTCACTCCATCACTAGAATTATAAGTGTGTGCGGCTACATGCAGCTTTTTACGTGGATTC
TGGGGACTGGATTTGAGTCCTCTTGCTTGTGTGGCAAGCACTTTGTCTCCCTAGCCCCTGTTCCCTCTTC
CTATTAACATCCTGAGTCGCTCCAGGACCAACATTGAGAGGGGCTATGGTGATGGCAGAGGACTTAGAAC
AGGCCTGCACTCTGGGGCAGTCAGTGGGCAGTCAGTGGACAGACAGGCCACCAATCCTCAGGCTCCGTAC
ACGAGGAGAGCAGTACAGGGGGAGACCAGGAGAGGGAAGGAGGGGGACTGGGCAGGCCTCACCCTGCATC
TCCTGCCTGCAGCTCTTCCGACGTGTGGCGTCAGCTCTGCCCGGCATGGAGAATGTCCAGGAGAAGAGCA
AAGAAGGGAGTATCCTTTTCCTTTATCATGTGAGTGAAAAGATGGCTCCCCAAGCCCGCGAGTCGTTTAG
TTCAGTAAGCAGATGAAGACCACCTTGCCTCTGTCGCAGCCCTCCCTGGTCTGATGCAGAGAGCCGGGCC
ACCTCTTTCAGTAGCCAGATGGGCTCCACGTTCTCTGAGCTCCAAACCTTCTCTAACCAAGGCTGCCTCT
TCTTTCTTAGCAGTGAGGAGAGACAAGGGCTGTGCAGCAGTGGGGGATTGGAGGGTGGTGGCTCTGTGTT
GCTTTGTAGTGAACACTGCGTGTGTTTCCTGAGTCCCCTGGAGATCCCAGACAAGGAAGCTTCCATGCAA
TGAGCACTTGATTTCAACCTGCCAATGCTTCCTCCATCTGCCCACTGCTCTGTGGGGAAAGCTTGCCCAA
CACTCATCCAGTATAACTAAAAACCTCGCTTACACCCAGATGATGAATCTGAGGTGCGTAGAGCAATCCA
AGGCCCGCTGTGTGTGTGTAAGTCAGGGAATTAGCCTTTACACTGAGACTTCCTGCATATCTACCCCTAC
TCCGTCCTGGAGGGTTGTCTCTCAAGGATCCCAGCGAGGCAGAGATTAGTAGCGCTGTAGGTGGCTGAGG
ACATTGCCCACAAGCAGGCTTTCTTTGAAGTTTATCAGATGGCCCTACCTGAGCAAAAACCCACCTGCAG
AGTCAAGAAAGGAGCAGGACCGATGGGAAGCCAAGAGGTGTAGTGACCCTGTGCCCACTGGGAGGGACAG
ATAGCCTGCTTTTATCCTGTCCACTCCAAAGGCAAAGCTGACATACTAGCATGCATGGCGGTCATTCATT
CATGTTCTCCCCGAGGCCCCGGTGGCACTCCATGCAGGATTCACAGTTGGGTCCCCCAGGCTCCCACTTC
AGCATGACAACTAACGTGGCGTCCTAGTGAACCTAGCGACCCGCCCAGCTTTAGGGAGAAGAGCGCTGGG
CAGATGGCAAGCAGATCACACAGGGCACTGTTTGCCCAGCTGTGGGGTGCCCCCATCTGGCAGATGCAGC
CATCCGTACCACTCCCAGGTGCTCATGGCTGGAGCCTCCTATGAGGCAAGACTGGAGCAGCCTGGAGCAG
ACTCTATAGACAGCCCAGCAGGGGACAGGTGGAAGCAGCTGCCACCTCAGCTTGGTGCTGAGCCAGCACT
TGTGTTGACTGATGGTTCAAACCCAGATCCTGTCATGATGTGTTAGGGATCATGCTTGGGATTGCATCAA
CATGAGGAACAAGCACACAGAAGTGCCACTTGGTGATATATGAACGGTTCTCATGTGGCCTTACTCCTTG
GACTGGTGTTGGGGGGGGGGGTGCAGAGCTTCTGTCTTCTGCTTCCAGGTCCCTAGAGGGTGTCTGCACT
TCACATCTGCTCTGCACAGGAAGCCTGCCCAGGGCCTGTCTAGCAGGTCCCCTTACCCACCCTCCAGCGC
TTGATCTCTCAACAAAGTTGAGCTAAGGGACCACAGAAAGATAGGGCCACAGCCCTTTGCTGTATCCACA
CACACAGGGCACTCCTTTCTGGAACAAGGGTTTCGTGTGCCAAATAATGACAGTAACTTGGCAGTAACTA
ATAGATTTCTCCACCAACGAAATGAACCAATTCAAGCCAGATCAAAAGTGTAAAGGCAGGTTTATTGAGA
GGCAGCTGTCCGACGAGGCACACTTGGAGGTCAGTAAAGTCACACGTCCAGGCTAAAGCAAGGGAGTTAT
GTGGGTTTTAGGCACAGAGTGATGACGTGTGAGCTAGGAGCTGGGATTATGCCTGTGTAAAGTAGAGAAG
CTGTGTGGGCACTCACTGTGCGTGGCTGGAAACAGAGACAGGGAGAGGGGTTGTGTTAGCCTGGAATTTT
CCTCTTGCAGGGCCGGGTTGGGGGAAGAACAAATGGGGTTTCTTAGCTTGTGCAAGGGATATGCAGGGGC
TCCAACCAACCTTCCTGACCTCTTTGGGTAGAAGGAAGGGCACTGATTCAGAGCTACATCCTGCTGCAGA
GGGAGAGAGTCGGGAGTCAGTGGGTCCGAGTTCCTCGGCAGGTATGAGTGCAGAAGGATTTGGTTGACAG
TTACTCGGGAAACTTTGAGAATCCGTTCTGGGAGAAAGCGGAGGATGCAAGGTGCTAGGAGGAAAAAAAT
AGATTAATACCACGGCCCTGTGAACGGGGCTAGCTAGGTGCAGAGTGAAGACTGCCAGGAAGAATAAAAC
GGAGACTACCCTGGTTGTCTTGGCAGGGTTCTTCAGAGTTTGTGAGGTGACTCAATGTTAGCCTCTACTA
GGCCAGGTTTGCTGGCATAACAACATTCTTTTCCCAGGGACATACAGGTGCCTCCTGAGGGATGAGCCTG
CTGTGAGGAGGACCAATGCTCCTCACAGGTTTTGCATGGCCACGTGAATTGATCTCATGCTGGAGGAGTG
GCCACCAAGTTGTTGATGGGCTTTTGGGGACAGTCAGACACCTCTCTGATCTCAGCCACCTGTATCCCAT
GCCCCCCGCAGTGGAGGTCACAGCAGCAATACCTGTTACTACTGTGATCTGTGTGGGCAGAGGGCACCTG
AGATTGGAAAGAGCTGGTAGGATAGGAGAGCGTGGCTGTGTCAGCCTTTCCATAAAGCATCTGGTTCGGT
ACTGGTGTCATTGGGATACACAGAGAGGAAGGATCACCAGGAGGCAAGCTTTTTGCTATAGAGCCGCTGC
ACCGAAGAATCCCCTGCTGTGCCTGGGGAAAAAAACAAAGTTGTGTTAGTAGTATTGCAAAGGGAGCCAG
GTCCAGAGTAGCAGAAATGGAAGTGAGTGTCTGGAAGAGGAGTGCGGCTTGTATAGTGATAGCCTTTGGT
TGGAGGGTCAGCCCTTGGCCTCACGAAGGGACCAGCAAGGGAACTGCTCTCAGAAGAGCGCAGCCCAGAG
CAGCCCAGAGAGAGCAGTTGTTGTTGTCAAAGACTTTAGAGAGGTTAAGTAGGATTGCTCCCTCCTGGAT
GAGGCCCAGCCAGGGGAAAGGTTCATTTTCTTTAGTTTTTACCTGGGGTTGGGAGGAGTCTCTCGGGTGT
GATTGGAGGGACTCCTGTTGTGGTGTGGTCTTGGTGAGGTTTGCCGGGAGCTGGAGGGAAGAGGGTCTGG
ATGTCGGGTCTCCAGATTTCTCTCTGTTCACATGGTTACGAGGATGTTTCGCTCTGATAGACTTGGCTTA
TTACCCCGAGCCCAGCGGTCATTCCAAGGATCTCAGACGCTAATATAGCTGTAGTTTAGTGGGGAATATC
CAGCCACTGGGCAGGCTCATCCCTGTCCATGCTCTCAGCAGCTTTCTTGTCTGCACCCTCCTCGGATGTT
CATTTTACAAGGGCCCCATGAGCAACCCCCTTGTTCTTTGACCCTTTGGGAATGACAAACTTCCTCCATC
TGGTGGTCGTGGGTAAAGCATTCATAGAGGAGCAATACTCCCTGTGCATTTAAAGAAAGATCTGGGATGG
AAATACCATCTTGGCACCCCTTAGAAGGACAGTCTGTTTGGCCCGCGATGTCGTTGTGGGTTTCACCGGC
TTGATCGCAGAACATGGAAGCAACAGCAGTATGTTGGAGCTGATTTGGTCTCACCTAGCTGGACAACCAG
GCAGGGGTGTCTCGGTGTCTGGCACCTGTGGCAGCAGAGAAAGGGTGTGACCCAGGAAAAGGAAGAGGCA
AATAGAGTGAATGTTGTCACGTTTGACCATTCAGACCTGGTTTTGAGAATCCCTGTAGAACCTGAGAGCG
GTGGGTGCCAACTGCTAAACAGACCAGCCATCTTGGGCTGGGGCCTTTTTGAGCCGAGATAGATGGTGCC
AACACTGATATCCTAGGAGTTTGGCAGCTGAGGGAGTGGTGATTACCATGTAGAGTCCGATCCACTGGAG
CTCTAAAGATTTGGAAGACAGTTGTTTGAGAAGCACCTGGTCCCCTGACCCCGGGAAAGGGACAGGGGTG
GGCAGTGGCTGAGTCTGCTGATATTGGGGTAGGGAGATAGTTGTCAGTGTGTGAGCCGATAAGGGAGCAC
GGGAGGGATAGGGTAAGTAGCTGGGGGAGTCCGGGCCCGCGGATGTTGGTTGAGGAGAAACAGCCTTCCG
TGCATGAGCTCAGAAGGACTGAGGCGCGTAGGAGAATGTGGGGTAGCTCAGAGGTGAGAACACTTGGGAG
AAGGGAAGGCCAGGGTAACCTGGGCTCAGGTGAGAGTTCTGAGTTGTTTGATAAGGCTCTCTCTACCTTT
CTCAATGACTGGGTGGCACGGAATGTGAGATTTTCGGGTTCAAGGCATCCCTGGCATGCAAAACTGAGCT
CCTGTGTCCCAGGAGCTGTCACATCCGCTGCTCCCCTGGAGGTGGGGAGCATTTGGATCCACCCTGTAAA
AGTGTCAACGAGGGTTAGGAGACGGCAGAGTCTTTTCTGGGCAGACATGTGTGTGAAGTCAGTTTGCCAG
TCTTGTCCTGGTTGGTGTCCTTGGAGCTGGTGCAGGGTCTGGCGTATCTGGAGAGCCCCCTGTGAGTTTG
TCTTGGCACATGTTTGACCGGAAGGATGAGGCTGTAAAATATGGGCTCAAAGGTCAGTGGAGTCAGTGGG
GAGGGGCAGCTTTAGAAATGGGAACAGAGCCTTGGGTCTGATGTGTAGGCTCTGGTGGATGTCTGAGATA
ATAGAAGGTGCCAATTTGAGGGGGAGAATAAAGCTGTTATTTAGGCAAAGCCATCCATCTTTTGTCTGTG
TGGCCCCTTTGTTAAGGAATCTGGCTTTATCCTCCATCTGACACTGAGGTTTGTAGGAAGATCGTAGGAA
GAGGATGTGCCCCGTGCGTCTGTAGGAGCTAGGAGCCAGACCTCAGACCACTGAGTCAGCCCTGGAATTA
TCCACAGGCTCTGGATCTGTGGAGGTCTGGTCCCTCCCTTACTCCTGCGCTGACTTATGGCCACCTCTGC
AGGGAGTTGGAGGGCTCCAAAAGCTTGGCTATAAGGGGACCACCCATGGTGTCAAGGAACCCCCTTTCCT
TCCATAGGACAGAGGGGCTGTGAGCTACAAGAAAGGCTTACTTTGAGTCAGCATTGGCTCATTGTCCTTT
GTCAACATGGAGTGCCCTGGTTAGAGCAATGAGTTCAGCCTTTTGTGAAGAGAGGTTCCACTGCGAGATG
GTTCGACTCAGTCATTTCAGCTGAGGTGACCACCGCGTATGCCGTCTGGTGGGTCCTGAGTTATCTCTAA
GGGAACTTCCATCCACAAAAAGGGTGAGTTGTGGGTCTGTGAGAGGCTGGTCTGGGAGGCTGGGTTGGGG
TGAGCGTAGGTCCTCCACAGCTCTAAGCAAGAGTGGGAAGGCACATGGAGGAGGGTGGACCAGGAGGAAC
AGCTGCTGGGTGTAGAGCAGGGCTAGGGGCCAAGGTGACCTGGGAGTTTCCCAGGAATAACAGATAAAAG
GGTTGGGCCCATGAGGGTCTGAGGAGAGAGAAAGAAGGGTGGCTCAGGAGATCAGAGAGGCAGCGGGTAG
AGTATACAGTAATAGGCTGGAGTAAGGTGATCTTGAGTGAGTTCAGCCATTGCTGTTAGGTCTCGCAGAC
ATGGCTGCCATCGCACACAGTAGGGTCCAGTTGTTTAGATAGCAGAGCAGTGGCTGGGGGGACTGGAGAG
ATGGCTCAGCGGTTAAGAGCACTGACTGCTCTTCCGAAGGTCCTGAGTTCAAATCCCAGCAACCACATGG
TGGCTCACAACTATCTGTGATGGGATTTGATGCCCTCTTCTGGTGTGTCCGAAGAGAGCTACAGTGTACT
CATATGTATTAAATAAATAAATCTTTAAAGAAGAAGAGGAGGAGGAGGAAGAGCAATCAATGGCTCAGTG
AGAGGGCCCAGTGGGTTGGACTAGGGCCCCAGCCCCTCGTCAGTAAAGAGGTGATAGGACTGAGTAGAGT
CTGAAAAGGCCAAGACTGAGGCTGCTAAAAAGGCATCTCAGAGAGTAGGGAAAATGATGGTGAGCTATAG
CTGAGTGGGTGAGGGGCCCACTGGGGGCCCCCTTGACTGCCTGGTATAGTGGCTTAGCCATATTAGCTAA
ATATGGGAATCCAGGGTCTAAAGAATCCAACCAAGTCCAAAAATGAAAGAATATGATCTGCCATAGTTTG
GGGCTATAGATCTCTCAGAACCCGCGCTTGTTGAGGGTCTTAGAGCCTCGGCTAAGTTGAACACCAAGTA
TATATTACAGTGGGAGAGCATAGTTGAGCTTTAGATGGGGAGACTTGATCCCGGGTAGACAAGGTAGTTT
AGAAACAAGAAGTTGGCCGTCTGGGACAAAGACAGGGAGGGGCTACAAAGTAATAGGTCATCTACATACT
GAAGACGTGTATTTAGCATTAAGGTCTAAGGAGAGAGCCAGGCAGTGGTGGCACACACCTTTAATCCCAG
CACCTGGTAGGCACTGGAGGGCTTACGGAGAGGGAGAATGGAAGAGTTGCAAGTCCTATAGGGATCAGAA
GTCCCTGACACGCGAGGTGAGTATTGATGGACTTGAGACAGTGACGATGGGGCTCAGAAGTGGGGAGCTG
GGGTCTGAAAGGGGAAAGAAGAGGGGTAAAACTAGGACCGGCTGGTGGGGCGGAGCGATTACCAGGGTGG
AGATATACCATACTTGGGGGTCAACAGGGTCTGGTAGAGTACATCCAGGGTAGTCTGGCGTAGGGATCTG
TGAGGCAACCAAAGGAAGAAGAAGATGAGAGTGGGGAGAGGTTGTGGGGATTCAGAGAAGTGGCTGAGGA
TATTGTGGTCAAGTAAAGGCACAGGGCAGACAGGCATGACTAGGAAAGAATGTGTAAAAAATGGTCAGCC
AAGGATATAGGAAAGTGGCATAGTCGGCATGAAAGGAGGGGATCCCATCCATCCTCATAGCAGAAACTGA
TGAAGGAACAGCGGGATCCGAGTGGGAGGTCAAGACAGAGAAGGTAGCCCCATGTCCACCCGGAAGGTCA
TGGCACATTACCCTAGGATCTGCAAGAGTGACAGGAGCCCTCGAGTCTGGGTGCCATCAATCCTGTGCCA
GGCCGAGGAGCCCTAAGGCAGGAAGAGTTTGTGCTACCTGCACTGATGGCTCTGGACCTTCGTGACCTGG
CCCAGAGGACAAGCCCACACTGGGGCATTCTGACTTCCAGTGGTAGGGCAGTGAGCAGACAGCATGGGTT
GCTGAGGGCTGGGGCAGTTGTGGGATCAGTGGCTTTGCTGGCCGCATTTGAAACAGGCCCTTGTTGGAGT
TGACATTGACGGGATGGGGTCTGCGGGATTGCCAGTGGCCTCAGGTCAGTTGCCAAGTCTTAGGCTTGGA
GTGCAACCTTTTGCTGTAGCTGAGCCTGTCGGGAAGACTCAGTTGCCTCCCCTTGGGCATCGAATACCTT
AAAAGCCATCTTTACCAATTCCTGTACGGGGGTTTGGGAACTATCTTCAGTCTCTCCCCGCCCCCTCCGA
ATATCTGAAGATGACTGGGAAATAAAATGGGTAGCCAGGACTGTAGCCCCTGCCGGGGAGGCCTGGTCAA
GGCAGATGTGTAGACCATGGTTTGTTGTAACCAGTTTTTTAATGGCCAGGATTTCATTAGCTTCCTGTGT
AATTTTGCAGAGATTGCCAAAAGTCAACAATCTTATTTGAGACCATTTCCATTCCCTGAAGGAGACAATG
GACCGTACTCTAATCGGTGGTCAATGTGACCTGGCTAATAATTCCACTTAGGCTCTGTGGTCAGCACCGT
TGTGTCACTCACAGGGACTGTATTGTCCACTAGGTGGCCCTGGTCTGCATACTGTCTGCCTGCAGCCTGA
ACCTGTCCATGTTCCTCAGGAGTGAGGGTAGAGCCTGAATGACATAAAGATCGTGGAGTTAGGTCATAGT
CCTGACAAAGGAAGCAGAACTCTTTAATATAGGCAGATGGGATGGCAGAGAAGGGGCCTCTCCACTTTTC
AGTCTGGGAGAAGTCTTGAAGGGAGAAAGGAACATGAACTCCCACAGCTGTTTCTGCTCCGGCCACCTCC
TGGGAGGGCAGAAGAGCTTTGGAGTATTGAGACTGCATAGGAGAGGCTACAGGGGAGGGTTCAGGGGAAG
TGAGCCAGGGAGTGCATGGGGATCAGGGTGTGGGTTGACCCCTAGGTGGAGCCTGTGTTGGTAATGAAGG
AGGAGGGACAATGGGAGGAGAGAGGAGGGGAGGGACAAGGGGGACAAGAGTTAGGGGGAGGAAGTTAATT
GTCCTTGGCTTTTGTCTCAGAGGGAGGGGAAGAAAAAGGGGTGGGTACCAGGGAGAAGGCTAGCCAATAG
GCGGGTTATCAATGGTGTAGGAAGGAGGAAGGAAGGAGGAAGGAGCAGGTATAGGGAGGAAGGTTGGCCA
ATAGACTCAAGCTTATGGTGCTATAGGAGTGGGAGTAGTAGACAGGGCTGGGGAAGGGGTTGGTGTGCCC
ATGATTCAAGTGGTCCCGCCAGGGTAAAGGGTGTCTTGTAGTCCAAGTCCCCAGATGAATGTGTGTCTGG
AGGTGTCACAGCCTTGGCCAACAAGACTTGAGCCATTGAACACTGACTACAGAAGGGCGGAAGTGCAGTT
CCCAAAACATTTGTACATAGGGGAGCTCAGGCCATCTGTCCATATGCCAGCAGAAAATTTCAAGATCATG
AAGGATGTTAAGTCAGTAGTACCTCCTGGTGGCCATTTGGACTCACTGTCCAAGGCGTATTGTGGCCAAG
CCACAGAATGAAGAGGAACTTAGGGTCCAAGACAGCCCCAACTTGGATAAATTGCATAACCGCCACCACA
TGGGCTACTGAAGATTGGGTTTAAACTTGGCGTTGTCTTCTCCCGGACTTGCCAATGATGCAGCGACCCA
TATGATAGACTGGGATCGAAGAGTGTGGGGTGGAGAAACCTTGGAAAGGACGTCTCTGAATCCAAGGCCT
CCCTAGAACAAAACGAATCCAAAGCCTGCCTGGAACAAAGACTGAGTCTTTGCTCTGAATGGACAGGCTT
GTCCTGGCTCGGCCGAGACTTGTCAGGGGATCCCTAGCTTGGTCCTGAACCTGGAAGAGGCACCCGGTAC
CTGTTAGGCTGTGGCTGGGAAGTGGCTCTCATTGAGTAGAGAGAGGGAGAGAAGTGGTCCCCACATGGCC
TGAGGCCTGACCCACTGGACTGGGGGCCCAGCTTCTCCCAATTCTTCAGAATCATAATAGGTTTCTCCAC
CAACGAAATGAACCAATTCAAGCCAACTCAAAAGTATAAAGGCGGGCTTATTAAGAGGCAGCTCTCCAAT
CTCATAGGTGGAGGTCAGTGAAGACACACATCAGTCAGGGCTAAAGCAAGGGGAGTTTAGGGGCACAGGG
AGATGATGTGTGAGCTAAGAGCTGGGATTATGCCTGTATAAGGTAAAGCATATTAGCCCAGAATTTTCTT
CTTGCAGGTAGGGTTGGAGGGAAAAGAGCAAACAGGGTTTCTTAGCTTATGCAAGGGATAACCAGGGGCT
CTAACCAAATAGTAACCATGTGTCCCAAGGAGAGAGTGTTAGGATTAGGCATTGGGGGTACCTTGGTGGG
TCTGGGCCAAGGTGTGCTGCTAAGAGATCACTCTACATAGCAGACCTCACACACAAGAGGTTTATGGGAG
AGGGGAGGTAAAAGGCCATCTTGGAGTTGAGATATAAGAAACAGACAGAGAGACTGGGAAACAGAATTAA
CAAGACACACAGAGAGAGGAAGATCAGGGATGGCTTGAGACTTAAAGGGTGTGTGTGTGTCACATAGGGG
AAATAAGCAGCCATAGGACGGTAGAAATGCACCACACCTGGCCACAGCGGGCAATGACATAAGCTACTAG
CTCTGAGTTGCTGAAGGACAAGCTGGTGGGGCTCCTGATTTATAGTATTCCTCCTTCTTGTTTATTATAA
AAGGCAAGAAAAGAGGAAGGGGTCGTGGTAAGGTGGCAATGGGGGTGCCTATGCTTTTTAGACTGCTCCC
TGCTATCTCAGAGCATGGATATCTTTGGGGACCCAAGAAAACTGGGGTGCTGGCCAACTCCTGAGAGTGC
TGGCCATTTCCCTAGCTGTGTAGGCTCTGTGGGACCATCTGGGGCTAGCAAAGTGCAGGCCTAGTTGAAG
CAGTCTGTGAGGCTGGACCACCTGGGGCCAGCCACTTTCAAGCTATCCTAGATGCTGACTTTGAGTTAAC
ATGGGGACCTGGCAGGATGCTGGGACAGATACATGCAGGAGAAAATCTTTTCTTAGGTGTACATTTGAAA
CTTTTAGACCCAGGGTCCTGGTAGTTGGGAGAGGGGAGTCCCCAAAACCAAGGAAAGGGGATCCCACCGT
CAGAAATAAGTGGACAGTAGGCAGGTAGGGGAACAGGCTGTTGACTGACAGTACTTATCTGGAGTCCATT
CACCAGGAGAGAAGGATTCATCCTATTCCCTGAAGCTTACGAGAACTTAAATTTACAAAAAGAAAGAAGT
TTTAGGCATGTTACCATTGCCACTGTTTGTAATCTACATTGAAATCCATATTGTAGAGAACAGACTCGCC
TTTCTGTCATAGTAGAAGCTCAGGATATGATTCTGGGAGGACAAGCTGGGGGAGGGCAGGGACCTTCTGT
GGAGCAGCAAGGCCAACACACACTTGATCTTGATATTCAGTACAAGTACAGACCATGAAAGAGGGGCTTC
CTCCCTCTGTCCGGAAGACTGGATGTATCTATCAGACAGACAGTTTTATCAGCGCAATAAAAAGCACCTG
TAAGTCTATACTTAGAAGACAGCCATAGGAAATCTAAAACCTTGAGCTTGTGACTGTTATAAAAGCAGTA
GTGCTTTTCCAGAGCTAGATTGATAGCTTATTAGAACTGCATTGACTCGGGTTAAAACTCTGGGTTTTTG
AAGTCTGAATACAACCATAGCACAGCAAAGGAAAGAAATCTGAAGCATTTTTTTTTAATATATACCTTGT
TACTTTCTTACGCCTTTACAAATTGCATTTACATGCCTTAGAAACACCTCCTCAGACCCTCAGACATTTG
CTTAGATCCTCAGAGCTTAAGCTTTAAACTTCACACCTTTTGTTCCTACCCAGCCGTCGACTGAGAACAG
TCCTCGGAAAGCAGAAATAGTGGAAAGTTACACTGGACTCTATCTTGTGAGTTTGACTCTTTTCTGAGTC
TGGTAACAAGGCACACTGGGTCTCAACCCAGTAGGAGCTCTAGGACAGTGAAGCCTGGGGCCTGATTTTT
ACACCTGAAGACAACTGAGCAGCGACTGAAGCCTTGGTTGCTGCTGTTAACAATTTACCAAGCTGTCACT
AGCCGAGTCCTTGGTGCCAGAGCTCTGAAAAGGATAGATTTCATTTGAGTTAAGTAGGAAGTAAGGCCAC
GTAGCTTCCGAATCAATTAAAAATGACAGAGACTTACCAGCTACCTGGACAGTCACCCTAGGGTGTCTCT
GTGGTCATTGGGGACAGAGGTCTCAGGGCTGTGTCAGACTTCACCTGCCAGGCTCAGAAGGTCCTGACAG
ATTGTCCTGTGGAGTAAGAACTTCGGGGGGCACTTGCCTACCTTGTCTAGGCAAAGGAAGGCAATCTGCT
CCCTAGTGTCTTACTTGTCCTCAGGTTGGACAGTAGCATCCTAGCAGCAGTAGAAGTGAGGGGCAGTTTT
GGGCCCAGTGTCCAGCCTTGCCACATTTAAAGCAAGCTCCAAGTGAAGGTTCTTCTGTGCCCATCATCTT
CTTTGAAGGAGCTTAGGGGTGCTGCCAGGAGCTGTCATAGCATGTCTCTCTAGCATGAAAACCTTTTTTA
AAATTATTATTAATTTACAATGCCATATTTTGCAGATCTCTGAAGCATTTGAGGACCATCTATTAGTATA
AATGATTTAGTGGTCAAAGGTTCTCCGAGAGGCATCGCAAAGGGGAGTGAGGAGGTAAATGACACAATTC
CCATGGTGACATAAAGGGGGAGGTCCAAGGCCTAAGGTATCCCAAGGACTTAGGCACAAGTTGAACAGTG
GGTCACCTCACACAGTTCTAGGACCAGGGCTAAAAGCCCAAGGAGGCACGGGATGCCTGGTACCAGGACT
TTCTAATGAAGCCAGAAGATGAAAACCATACTCAAAAACAAAGCGTCATCCAATGGGAGTGATCAGGGGT
GGGTTGCTCTGCCAGAGACTCTGCCAGTGGACAGGAGGTATAGCTCCCAGTTGAGTATATACTTGATGTG
TCTGAGGCCCTGGGTTCCAGCCTCAGTCCGAGTAAGTAAATAAACCAACTCCAGGTGGGAAGGTCACGTA
GCTGTGGGGTTCTCCCAGGCCCCTCAGCCTCCTGTAAGGATGATGGGTGAAGTCTGATGCTGAAGTTAGG
TGGCACGTCCACTGGACCTCAAGCAGTGCTAGGGACAGAGTTCACTGCGACCTGAAGAGGCTCCACAAAG
GAACCTGGCTTAGAATGAGGCCACTTAGAGGCAGAGATAGGGAGGGGGCCTGAATGGGGCAAGGCTGTGT
GCGGTACTTCTAGATGGAATCTGTTGAGGAAGGGGACAGTCTTGATTGCTGGGACCTCCATGTATGGTAG
GGGGGCAAGGATCATCAAACCAGAGCTGATTCATGGGAAAAGACTGGATGCTAGTATGAGAGATCTTGGA
TGCTGTGCCTCTGCTCCAGGCGATGGGTGTCACTGAGCCGGCTTAGGGATGCCATTCTGCTGAGCAAGGG
GTGAGGTGGCTCAGAGGGGCACCTGGGAAAGATGAGAAGACCCCAGTGAGGCCTGTGCTCGGGACCAGCA
GTGAAAGCCAGGACCTGGAGTGTCCTGGGATCAGTGGGGACCACCACAGAACCACAGTTAGCAGAGACTA
GCATCACGGTGGTCTAGGAAGCGTTTGTAGAGATGTAGGGAGCAGAGGAAAAGGACCATGGGATGGGAGA
GGGCAAAGCTGAGCTGAGGGGGAGGGGGAAGGAAGCAAGTTTACTTATGTGTACTCTCCAGTGGCATCAG
GCATGTGGGGGTAAGTACACACACACATACCAGCAAGGTAGCACTGTATGTTTTTCTGTAAGCACACATA
CAAGATAGAAGGTAGGGAGAAAAATCAAACTGAAAATATATACCGTGCTGTGTGATTGACAGAAAGCATA
AAATACTGGCTGCAAGCCAAGTAAAACAGCCAAGGCAAAGAGACAATGAAATTCAGGGTATGTTATGCCA
AAAGAACCTCATATGTTACCCCCAAAGACGGGACACAGAGACATCCTTAGAATGCTAGCAGCAAGCTCCA
TTGCCACCCCACCCCACTCAGCCCAGCCAAGGATCTGGGTGGGGCTGGAGAGCATCCAAGGGCTTGGTGC
ACAGTAGTGGTGAGATGTCCTGGCTGGAGGCAACGCTAAAGTCTTGTACAATCAGTTAGTCTGGGTTTGT
AGCCAGAGATGATGGGAAGGTACAGGCTGCCCTGCACTTTGCATCCGCTCCTCTGGTGTTCTTGGCAAGT
GTGATTCAGCCCCCAGCCCCCAGAATGCTGTAGAAAGGGGCTGTTTAGTCCGTGGATGCAGCTTCTCACT
GTAACGGCCTTGAGAAGTAAGGACCACCACCTCACATCCTCCTGTACTTTATGATATGGACACTCAATTC
TGATCCTGGCCAGGGGACTCTTCCCTCCCAGGCGCTTTGGGGGAGGGCCAGCCGTTAACCAGCTGGTTGC
TAACCATCTATTGCTAGATGCTGGAGTTGAAGCACAGTTTATCTGTCACCGGAGGAAGCTTCTAGCCAGA
CTCTGCCTGGCCTCTCTCTGTGTGGTGTTGATAGTGAGATCACAGCCCGGTTCACACTGGGTCGACAGTT
TTGAGGGGCGGGGTGCAGGCAGGTAGAGACAGGAGAGAAACGGGTGGGAAGCAGTTCCTTCCTACTCCAG
CCGAGCGAAGAGCTGAGGCTGTGGGTTTGCAGAGAGAGAGCATGGCGTCTTGGTGAGCGGCACTTCTTCC
TTAGCCCGCCTCCTACAGTGATTGACATCAAGCTGGACAAGCCTCAGGAGCCCCCGGCCAGCGAGGGCGG
CTGCTCCTGTTAATGCACCGCCCACTCGGCTTCCTCAGCGCCACTCACTTGTTTTGCCTCCTATAGCTTC
CTAACGGGGTGGGAACTGAGTTTATCAAGACTGGAGGATCTTTCTTTTCTCTCTCTCTCTCTCTCTAGGA
GTAGGGTGGGACATGGGGAGGGAGGCTGGGCATCAGGGACCACGTCACTCTTAACAGCTGTTACCTAAAC
AACTATTTTTTGGTTTGGTTGTAATATATTGTACTTTATTAAGATTGCCAAAAACTGTTAAAATTTAAAA
AAAAAATTTAAATCATGTGTATACAACTTTTTGCCAGATTAGAAAAGTAGTCATTTTTATTTGAAAGAAG
TGCTTTTTTGTTTGTTTGTTTTTGTATATTTGTAAACTTACAGAGAACCTCTCCACACACTTCTTCCTTC
CTGTTCTCTTGGAACTGTGTGTCACCTCTATTTTCCTCCTACCCCCGCCCCCCCCAGCCCAGAAAATTAA
AGCAATTAACTGAACAGTAGTAATTACATTCTAGTTCTGGGTCACATGGCCCAAACCAACCAAACCCGCA
CTCCACAGTCAAATCCAACTTTAATCTAACAAAACAGCCTCTGAGGGGGTATTGTCGAGTTCTTCCCTGG
CAGAGTGGGACACATTGGCAGGCCCCTGAGAGGAGATGAAGGCCCGTGGACTGTAGATTCAGCAGCAGCT
CCTTGGCTGCTTCCGCCTCTTCTCCTTCCCTGAAAAAAATAACCATAGAGCCAGAAAGCACAGGCAACAG
TGTGTTCTCTGGGCTGACCCGGTGACAGACAGCAAGAAGTTACTGGAAGGATGGGGTGCGCTTGGCAGCT
GAGGGCTGAGGCAGTGTGTAATCTACTGGCCCAACCATTGACACACATGGTTAGGCTGACTCGCCAACTC
AATGTGTGATGCTGGGTGGCTCAGGGACACTGAAGCCTAGGGCACCTGCCATCACTGCAACTACCATTGA
GGAAGTGAGAGGAGTCGGCCATGACTCCGGACACTCCATGTTGTCAGACCCTTGGATACTAATCTTGCCG
AACGTTGTTCTGGAAAAGCTGATTCCTTGATGTGTCCTCACCTCTGGCGTGGTACGTTTGAAATCTTTGT
ATGAATACCTTTGGGAGTGTTGGCAGCTGCACTGCACGCATCTTGGAGTCACTGTGGGGTTTCCAGAAGG
CTGCTGCCCCAGGGTACTGCAACTTGCCCCCCAGCAGGCTGTCCTTGCTATTTCCACTCATTCCCCAGAC
TGAAGACCTTCAGAGTCTCTTAGCAGAGTGCTCTTCTCAAAGCAGAATAGCTCTTATATTCTAAACGCTC
CCGGACTCACTGCCCCTGTCTGCCTTTGGGCAGGCCTGGGGCAGTGACTCTAATCAGAGCAGCCTAGCCC
CATAGCATTGGCTCTTCTTTCAGACTGCTGGTGGGCAGAGTGAATTGCCTCCACAAGGCAGAACTCCAAC
AAGGAGGTTGGGATGAATTATGGAATCTCTCAAAGAATGAGTTCAAAATGGCAGCCTGTGTGGCAGGGCT
GATTCTGTGAGTGGGCCCGTCATGCGACTCCATCCTGGGAGACTGTGTCAAAGCCGGTTCTGATGATGAA
GAGAGGAAGCAGGACCGCCCTGGGTACCTCCTCCAGAAGGCAAGGACCAATGTACGAGCATAATCGAAAC
GTCACGTGTTTAGTTTTTATATGTGTGTACATAATTATACATAGAGATAGACTTGTGTATACACCCTTCG
CGCGCACACATACACACACTCACATCCTCACATATACATGTGCTCAGTGTATATCCTCGTCTCTCCCGCT
CACAACTTGGAACTTACATTGTGATTCCGGTTTTTGGAAGTTCAAAAGTATGCAGTGTATTGAAATAAGA
AATAACTCAATATTGTCAAATAACTTGTGGTTGTCTTGCCTTCAATAAAATAGTGCATAGATGTGTTAAA
CTACAAGCAAAAGTAGTCCACAGAACTACCTTTACCACTGCTTTTTGTACCTGTGTGCACAGGTGTGCAT
GCCGTCGTGGGCCAGTGTCCCCCACTGTGGACACCCCACCCATGTGGTGCTTGTTGGAAGAAAGCATACT
TAGCCCGTGGTGACAGCAGCCTGGTTGGCAGTGAGGGGCTTTGCTGGTGGCCTCTGGCAGAGCCCCACTG
ACAGCACTCTCTACTGGCTTTCACTGAATGAATGTAGGGTGAATTTTAACCTTCTGGACACTGAATAACC
TTTCTTGTATTCCTGTAACTCGAATTAGAAAATCCGTTCACTTCTCTTGTGCACACAGTCTAAGACATCA
TGGCCTGCTACTAGAAACAGCAGGTACCATGGCAGATGCCATTTCTGGTCGATCTGGCCTAAGGAGCCCT
GCCTTCCTAGCTTGTACACAAAACCCCCTCCTTGAGTTTAGACTCTCCCAGCCCTGCGGCCCCTGAGAAT
TAGGGTTCTGCCCCATTGCTCCCCCACTCCAGAGGTGCCCTGGCCTTATTTCCTCAGCCAAGCTACCCAT
CTCCTTCCCAAAAGGCAATCAAGGCAGCCAGGCCCCAACAGAACTAACTGTAGTGGACCTGGGCTTTCCC
CCTGACCGCCACCACCATCCTACTTGTACGTAGGATCCAATCAGGTTCTCATAAAGACATCCTGAAATTA
TCTGTGCCCCCATCTTTGCTAAATTCAGATCAGTCAGAGGACAAAATGGAGGGTTCCTCTTAGGGCATGA
TCCCAATGTCGTAATTCAGTTCCTCTCTCTGGACACTGCCTGGCCTGGCAGATGGGCATGGCCCTCCTCC
TTTTCTTTAAGAGACTTGAAAGAGAGTTGTCAGCAAAACTGTGAGCAGTGCAGAGATTGTGAAAGGGCAG
GTAGTGTAAGTAGTATTGTTGTGGAGTCAGAGGTTAGAATGCTGGGTGGTTAGAGCCTTGCTGCTGCCAG
TCACTGCTCCCCGTTGCCATCTTTAAACATTTCGCAATATCTCAAGATCCAGGAAGGCATGGCCATGTCA
GTTCAATGTCTCAGGCAACAAAGACAAGGGTTGCAAGGTGGTAGCTAGGAATATAGACGCCTGGGCCTAG
TCTCTGTAACCCAGTGGCTAACCAGTGTCAGAGATCTAAGCAGGGCTGGCTTCAGCCTCCTGTAGGCAGG
GCAGTTTATCCCTTTTCCATATGGATGGGGGAGAGGCTGTCAGCAAAGTGGCTGAAGAGAGCTAAGTTAG
AGCTACCCAAGAGCCAGCCAGCAGGAGTCTGTGTCAAATGCTCAGGGCTCGATGCTCACTAGTTTCTAAA
CTACCTCTCCCCCAAGAATGGGAGGCACCATCACCTTGCCACAGCACTGGGAGCCAAACCCTTCCTGTTT
CACGGGTGGGAGAACCAGAACAGAAAAGGTCACCTGCAAGCCAGAGAGGATCAGAACTAGTGTTGGCATT
CTTGATCCGTTGCTGACCTTGCGGTGTCCAGACAGGACTGCTGTGCTAGCGTCCCTTAAGCCATCTCTAC
TCCATGGCTAGTACCTAAGGGTCTTCCCAGCCTGGCTAGCTAGAGAGAAGATAGGTGCATTTTGGGCTCT
CACTGTAAAGAAGAGTGGTCCCCAAATCTACTTGCATTGTCTTGTGTACACACACACACACACACACACA
CACACCCCAGTAGTCAGGGTACCCTTTTAATTTTTGTTTGAAATCAACTTCTTCACTGTTTGGAAATCTT
GCAGTTTCAATGTGCACACTCCTTGGACTCCAGACCCTGTCTGATTGATACTCTCTGCAGGGTATAATTA
TCAGGAAATCCACGCAGCGGGAGTTCAGATGGGCCCTTCAGGAGGCAGGAAGCCTCCATAGCAACGAAGG
CCAGCCTGCCATTGTGATGTGTTGACTTCTCACCTCACCTGTCCTTTAAAGTGAGGAGCAGAGGGCGGCA
TTTCTATCTGTACTGGGCTCAGAAGGCACAGTGGCCATATTGACTTTACATTCAACTGTGTAATATGTGG
AAAAGACTCAGCATGTTGGTCAAACAATAAACTGTCCTAATTCTCGA

An exemplary DNA sequence of the Rab6b gene in humans is provided below.

(SEQ ID NO: 25)
ACGCGCGCAGCCAGCCCTTCCCCCTCCGGCTCCCGCACCGCCGGCCGCCT
CCCCTCGCCCTCCTACTCTCCCCTCCCTGCTCCTTCGCTTTTTCCTCCTC
CTCCTCTCCCGGCCCCGGCTGCCAGCACCATGTCCGCAGGGGGAGATTTT
GGGAATCCACTGAGAAAATTCAAGTTGGTGTTCTTGGGGGAGCAGAGCGT
CGGGAAGACGTCTCTGATTACGAGGTTCATGTACGACAGCTTCGACAACA
CATACCAGGCAACCATTGGGATTGACTTCTTGTCAAAAACCATGTACTTG
GAGGACCGCACGGTGCGACTGCAGCTCTGGGACACAGCTGGTCAGGAGAG
GTTCCGCAGCCTGATCCCCAGCTACATCCGGGACTCCACGGTGGCTGTGG
TGGTGTACGACATCACAAATCTCAACTCCTTCCAACAGACCTCTAAGTGG
ATCGACGACGTCAGGACAGAGAGGGGCAGTGATGTTATCATCATGCTGGC
GGGCAACAAGACGGACCTGGCTGATAAGAGGCAGATAACCATCGAGGAGG
GGGAGCAGCGCGCCAAAGAACTGAGCGTCATGTTCATTGAGACCAGTGCG
AAGACTGGCTACAACGTGAAGCAGCTTTTTCGACGTGTGGCGTCGGCTCT
ACCCGGAATGGAGAATGTCCAGGAGAAAAGCAAAGAAGGGATGATCGACA
TCAAGCTGGACAAACCCCAGGAGCCCCCGGCCAGCGAGGGCGGCTGCTCC
TGCTAATGCAGAGCCGACCTGTGGCTTCCCATGACACTCCTTGCTTGTTG
TGTTGCTTCCTATTGGCTAGCTTCCTAAGGGGGGAGGGAACCGAGTTATC
AAGATGGGAGGATTTTTCTTTTCTCTCTGTCTTTAGGTGTAGGGTGGGAT
GGGGAGGGAGGCTGGACATCAGGGATCACATCACTCTTAACGGCTGTTAC
TTAAACAACTATTTTTTGGTTTGGTTGTAATATATTGTACTTTATTAAGA
TTGCCAAAAACTGTTAAAATTTAAAAAAAATTTAAATCATGTGTATACAA
TTTTTTGCCAGATAAAAATGTAGTCATTTTTATTTGAAAGATGTGCTTTT
TGTTTTTGTATATTTGTAAACTTATAGAGAACCTTTTCCACACACCTCCT
CCTTCCTGTTCTCTTTGAACCATTCATCACCTCTGCCTTCCTCCTATCCC
CAGCCCAATAAATTAAAACAATTAACTGAGCAAATTAATTAGGCTTCAGT
CTGGGGCCATCTGGCCCCACTCTCTAGGCCCTACTCCAGTTAAATCAAAC
ATTGGTTGAACACATCAGCCTCTGAAAAGGTAGCTCTGACTCTTGTCTTT
CCATGGCCAGAGTGGGTCGTCAACCTCGTGTGGAGCTGAGACCAAGGTCT
GTGGGCTCTAGGCAGCCCGGCAGCAGCCCCCAGAGGAACTGCTCCCACCT
CTCCTTCTCTTTCCCTGAAAAGCCTTGAAGGGGGTTGAGGGTACAGGCAA
CCTTGTGTTCTCTGTGCCCTCAGGGACTGGCAGAGCAAGAGGCCATCAGA
AGGACGAGGTGCTGTTGGCAATTGCAGCTGCGTGGTGCGTGGTTTATAGA
CTGGGGTGCCATGTGTACTTCTGAAAATATTTTAACCAGCCCACCCAGCA
GTCAGTACACATGCCTGGGCCAAGTCTAAGAAACTCAGTGTGTGATGTTG
GGCAGTGTGCCATCCTGAAGCCTTCCTCCTGGATGTCAGGGAAGCTGGAC
CACCTGGAGTTGATCCTCTGTGATGGGGAGGCGGGGGGGGGGGGGGGTGT
TGTCTGCAGAACCACCACCTTGAAAGATCCATGTTCTTAGAGCCTCAGCT
GCTAATATTGTTGAGTGCTTTCTGGAAAAGCTGATTTGTTGCTATGTTTT
CACCCCAGTGGCATGGCGCAGTTGAAGTCTTTGTGGACATATCTTTGTGG
CTACTGTGGCTGCGCTGTGGTCACCTGAGGATGTGGTTCAGGTGGGGTCT
CCAGAAGGCTGCTTGCCCCCAGAGAGCCATCTCTTCCATTTTTGATTCAT
TCCTGAGAATGGCTTAGTCCTGAGAATCTGCAAAGTCTCTGAAGAGAACT
CTCTCTTCCCACTAAGTGAGTCTGGCCAAAATGTAAACCCCACCCTCCCT
GCTGTCATTGGCTCTCCCTCCAGGCTGCTGCTGGGCATGTCATAGATGGG
ATGAATGACCATCTGGGGTATGGTGGGCTGAACTCCACAAGGTCGTTGGA
ATGAATTTTGAAACTTCTCAGAAATTAATCTGAAATGGCAGGCAGTAAGT
GCAGCAGAATCTGGCAGGACTGATATTTGTGCAGTGGGCCTGCCACACAC
CCTCTGCCTAGGAGGCTGTGTGCCAGAGCTCATCTCTAGCAAGGAATTTA
TCTTCAGGTGAAGAAGAGAGGAAGCAGGATCCCACTGAGCACCTCTTGCA
GAAGGCAAGGACCAATGTTTGAACGTGTTTCAGATGTCATGTGTTTAGTT
TTTATATGTGTGTACATAATTATATATAGAGATAGACTTGTGTATACACC
TTCCACAAACACATCCTCACGGAGATACATGTGCTCAATGTATATTCTTG
TTTCTCCTGCTCTCAAGTTGGAACTTGCATTGTGATAAGTTTTGGAAATT
CAAAAGTACTGCAACGTGTTGAAATAAGAAATACTTAATATTGTCAAATA
ACTTGTGATTGTCTTGCCATCAATAAAATAATGCATAGTTGTGTTAAACT
ACAGGCAAACATAGCCCACGGGAATATCTCCACCATTTCTCTATGTACTT
TATATGCACGGGTGTGCATGCCTGTGTGGGCCAGTGTCCCCCCACCATGG
GCACACCCCCAAGTGGTGCTTGTTGGAAGAGTGCTTACTTACCTCGAGAG
AATTTACAGGAGTCCACATGACAGTGTCCTGCCTGGTTGGCAGCGGGGGC
CATCACCTGTGGCCTCTGAGACCGCCCCATTTACAGGAATCTGCACTGGG
TCTCAGTGAATGAACGTATGGTAGATTTTTATTTTCTTGGCACTAAAGAT
TAGCTTTTTTAAAAAAACATCCCATAAAACAGTAGAAACTCAGTTCCCTT
CTCAGGGGCACACACATCCAAGGCACCGTGGACTGTGTTTCCATCCTGGC
CTAGAGGAGCAGTAAAAGATAGCATTTGGTTTGACATGGCCTTAGTGAGT
CCTGCCTTTCCTAGCATGCACACAACATCCTCTCTTTGAGTTTGGGTTCT
CCCCAGCCCCTTGGCCTCTGAGGCTGAGGGTGGCTTCCTCCCATCCTCCC
ATCTCAGTGGTTCCATGGCGCTCCCTTCGGCACGTGCGGTTTCCGCCCCT
CCCTTCCTGGGGTGTGGAAAGGCAGCCAGACCTCTTTTTCAGAAGTTGTT
GGTTCCCTGATTTTTGTCTTGAGCAGGCACCATCCCTGTCCCCTTGCTTG
CAGGTAGAATTGGCACAGAGTTAAGGTTCCATAATAAACTGTTAGGTGCT
CTTGAAGAAGATGTCATGGCATTGCCTCAGCACCCCAGCAAGAGCATTGC
ATCTTTGCTGAATCCAGATGGGTTAGCAGAGGGCAAATTGGAGGATTCCT
TTGAGGAAATGTTCTCAATTGAGCAATTCAGTCCCTCTCCCCAAAGACTA
GACGTGACCTGGCAGATTAGCAGGATCCTCTTGGCCTTCTTTTTTGGGGA
TGGTGGGAGTGAAGGGGGTGAATCTTAAAAGCTTATCAACAAAGCTCCTG
TTCTGCAGGCATCAGGAAAGTTGAGTGTAAGATGGAAAAAGCAAACAGCC
TATTTACAAGTGGAAACACATTGGTTCTGATCACTCCTAACTTGCTGGGT
GATCATACATACATCAACATGAAGGTGTGACATCAGAATCATGAGCACCA
TGGCTGCAGTTGTGTGATCCAAGGCTAGTTGGCTTGGTAGCTGTTGGCAG
CAGTTTCCCTTGGTCAGAGGCCTCCTTGCTATCTATCGCTGCTCTCTTCT
TGTCATCCTTAAACATTCAGTCATGTTCCCAGGTCTGAGGGGAGCAATGG
CCAGACCAGTTTCAGTATCTCTGGCAACAGAAACAAGGATTGAAATGGTG
GTAGTTAAGAGTGCAGACACATCTGGGGTGCAACGTCTGCATCATCCTGG
TGGCTAGCCAGTGCGGGGAGGCCCTGCCCTGGGATACCAGTTGTGACAAT
AAAGGGATGGCCCTCCTGGCTTCAGCATCCTGCAGGTAGAGCAGCCTCAT
TCCTTCACAAGATGGAAGGCAGGTGAGGATGTCAGGATGAGATGGGACCA
GGTAGCCGAGGACAGTGAGGCATGTCTGAGCCACTTGAGGGGCAGCTGAA
CAGAAGGATGTCTGTGTGCATCAACCGAGCAGATTGTGCGCACACTCGTT
TCCGAACTGCCTCTCCCCAAAGAATGTGAGGCATTGTCACCTTGTTACAG
CTATGTGAAGCAGAGCTGTCACAACTTTTCCTGTCTACAGGCAGGAAAAC
CAGGATGGAGAAGGGCAACTTGCTTATTAAGATCACCCAGCAAGTTAGGA
GTGATCAGAACCAGTGAAGTTTCCATTTTTCATCCATTGCTGCCCTGTGT
GTGAACAAATGTGACTCCATATTTATTACACAGCAAGGACTGCTGTGTGT
GGGTGTGTTCCTCAGACCATCTCTACTCTTTTTGGTTAGGACACTAGGTT
CTTCCCAGCATATGAGTGAGAATATTGGTGGTATTTTGGGCTCCTCTACT
GCACAAGGGCCAGTCCCCAAGTCTACTCTGATTGATTGTCATGGGCACCC
ACACAGATCAGGACACCCTTTGAATTTTTGTTTGACATGAACTATTTAGG
TGTTCCAAAACCTTGGGATCTCAATGTGCAAACTCCTTGGACTCCAGACC
CCTGTCTGGTTGGCACTCCCAGCAGTGTGTGGTTTCATCAGGAAATCCTT
GCAGCAGGGGTTTAGATGGTTCCTTCAGGAGGTGGGAAGCCTCCATAGCA
TTGAAGGCCAGCCTGCATCTCGCGGTGTGTGTCCACTTCTCCCCTCACTT
TCCCCTCACCCATCCCTTTAAAGTGATTGGCTGTGAGGAGCACAGAGGGT
GGCAATTCCATCTGTTATGGGCTCAGAAGACATAGCCGCCATGTTGATTT
CACATTAAACTGTGTAAAATGTGGAAAAGACTCAGCATGTTGGTCAAACA
ATAAACTGTCCTAATTCTCAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAA

n-R5s89, or nuclear encoded rRNA 5S 89, is a ribosomal RNA found on chromosome 9 in Mus musculus strain C57BL/6J chromosome 9, GRCm38.p1 (NC_000075.6 (GI:372099101), the sequence of which is incorporated herein by reference.

Gm7364 is a predicted gene in Mus musculus that is found on chromosome 9 in Mus musculus strain C57BL/6J chromosome 9, GRCm38.p1 (NC_000075.6 (GI:372099101), the sequence of which is incorporated herein by reference.

The 1 Mb region on the chromosome 9 between the position 102,450,352 and 103,338,569 as stated on mouse genome GRCm38.p1 is provided below (SEQ ID NO: 14).

Nucleic acids in BOLD and underlined denote the Marker used to delimit the chromosome region.

AAGAAGCAAACGGGAGTCCTTAAACTGAGGCTCATTAGACTTAAGTTCAGGAGGGTGTGTGTGTGTGTGTGTGTGTGTG
TGTGTGTGTGTGTACATATGTGTGTGCATGTGTAAGCATTGTGCCCATGCGTGCATGTGTATGCTTATGTAGACATGTA
TGTATTGTGTGTATGTATACATACGTATGAATTATAAAACAAAATAAATTACATAGCATAAACGTCAGAAAATGCATTT
CCAAAGCAGGTATCCAATAACTATGTGGTGTGATTAGCGTTCTAGGATATGCCTCTTTCCCCAGACCTTTAGCTTGATT
GCTTTTTTAAATTGTTGTGGATATATGTGTGGGAGGTGTGTGTGCAGGTGTGAATGCATGTTCACACCTGTGGGAGCAC
ACACAAATGTGCGTGTGAATGGAGGCCAGCAGCTGAGTGAGTGTCTTCCTCAATCTCTCCTCCCTCTATTTATTGAAGC
AGGGTCTCTCACTCACCAGAGATAGCCAGCTTGCCCAGGGGATCCAAAGATTTGGAATTACAGTCAGACCACCATACCC
ACTCAGGTTTGCTTTTTCTCTTAATGTAGGAATTTCTGCCATCACACCTTCATGCTAAATGAATCTACTGATTCATCTT
CTCAGCCCTCCTTCTTATGTATGACTCTATCGTATAACCTATACACAGTCCAACTACATTCATGTTTCAGTAAAGCACA
AAGCTTTAAACAATATGTTCTGTAGATACCATGTTTGACTTGCTATCGAAACAACTGGGAGTTTTATCATGAAATGTTG
AGACAAGTTCTTTGTCCCACGATGAACTTATCTCTACGCACGCACACACACACACACACTGTTCATTATATTAAATTAG
ATCCTATTCAGATCAGCATTAGCATCTCCCACTCTCCCAAGCGCTCTTTCGCCGTATGGAGGATTCTGTAAAAATTCTT
CTTTCTTCCTCACAGGAGAGTTCTTGAAACGGGAAGAACAGCTGCTGTGTTCACGGGTCCTCCTTTGCAGATGAAGGAG
CTCCCACTCTCCACGTTCCTCAGGGGAACTGTCCTCTGGACCCGCCAATCACGCTGGGTGTATGCCATTGCAGCTAAGC
CTCTTGAAGCTAGGCTGGTGTTTCGACAACGCATGTTGATGAGGTGAGTGGTGGCCTTGGCTTTTATTTCTAGAACTTG
ACTTCCAAACATCCATCTTAAGGAAATCATCACACAGTGGGCCAACGTACTAGAAAGCAAGTGGGTAAAGTATGTTACA
TGCGCATAAGATTTAGCATTGCACAGCTATTGTCTAGAGAACAATTGTTACCCATTTCCCATGTGCAACACTCTCATGT
ACACACTCACATGTATGCATATGCACAAGCATATGCTTTTATAAAAGAAAGAAACATATCATTAGTGGTATTATATGAT
AACTCTTTTATATTCTGTATGCATTTCTCAAATATTCTACACAGGATGTGGAGTACTCCTGATTCACACATACACACAC
ATACACACACACACACACACACACACACACACACACACAAATAAAAAAATAAGTTAAAAAAACAAAAACTGGTGTGGTG
CCGCATGCCAATAATCCCAGTACTCAGGAGGCAGAGGCAGGAGGATCAGGAGCTCATGGCTTGTCTCTGCTGCAGAGTC
TGAGGCCAACCTGAGACCAAGTTTCAAGTTTTCAAAGTCTTTTTTTGAATTTAAGGATATTTAAAATAAAATGAAACAG
AAACTATTTAAATAGAAGAAGGAGGAGGAGAAGGAGGAGGAGGAAAAGAAGGAGGAAGGAGGAGGAAGAGGAAAAAGAA
GCAGCAGTAGAGGCTAGGAACTGGACCCCCTAGTGCCTCCCATTGTCTGGAATAGCACAGGAAGCTGGTCTTGTCCCAG
AGCCTGGCTGGTGCTATAGTTTCCTACTACGGTGACTTTCAACCAGCAGCCCTGAGACACAGCACTGCAGGAGTTGCCC
GTCACCACCACTGTGTACTGATGGCACCATGACTGTGACTTCTTTTACCCCAGTCAGGAAAAGCACAAGCAACAGAAGG
TGCCCAAGACTGAGGAAACCTGAGTGAGTGAACAAGACACTGCCTGCAGCAGGGAGTCCCTATGTCCCCTTAGACACGG
GCTTTCCCAGTCATTACCATACATTAAGGATATCCAAGTGAACAAATCTCACCTATAACATGTGACCTCCTAGTCTCTA
AAACAGCCTGCACAGGTGTTGTCTTAGATCAGGTGTGTCCAACCTATTGACATTGCATCATGACTCATGGTTATAGCTT
TCCTAGGAGGTATGTAGCCTAGAAGCCACAAGTTAGACAAGGCTAACACAGGTAGAAAGCTCTGGATGCCTTGCTTAAG
AGGTGATCAAAAGACCAGGAGCACTGATTGGCCATATCTTACCCACTGTCGCTGAGCTTCAGTTTCCACCAGTAGAAGA
GAAAGACTCTACTAGATCCATATCTCTGGGTCACTGTTGGAACTACCAACAACTAGTGGTCATAAATGACATGTGACAT
GTCACAAGGAGACCTATACTGCCTGTCCTTTCCTGGAGCATTCAAATGGTTGTCCCTGCAACCTTCTTTGTTCCTCTGT
CTTCCAAGAAGTTTCGCATATGTGGATGACCTGTAGGCATAGAAGTGCTCATATTCTGGCCTTATGCCATTCTCTAAGA
CAGATCCAGAGCCCCTTCTTTCCCTCTGGCACTCCCTGGGACCCTTCTTGGACTCCCTGCTGTGCAATGCCTCCGCCCT
CCTTCTTCCCCAGTCAGTCATATGATGGGGCTAGCGCCAGCCAGGACCCACATCTGCCTGCCCACAAAGACCAGATGGG
CCACATGTTCTCTTGTGAGCCAAGTGTCTGGTCTGAGAAAGCCTGAAAGGTCATCACTGTGTATGGGCAGGTCCCCCCT
TCAGCAAAGTCATCCCTCAGTAAACGATGAGCCCTGCCACCTCTTGCTGGAACAGTCAGCACACTGTATGCTCAGCCTT
TCCAATCAGTTTTGTTTCCACTGTCTTTGGCTACTGTGTGTGTGTTGGGGGCGGGGGGGGGGAACGTTTAGTGTAAAAC
GAAATGGGATTTTCCCTTCGATATTCTTCTTGGATGACAGGTCTGTGATTGGAATCAGGGCATGAAAAATCATGCCCTG
GTGGCTGTTTCCATTAGTGGCTTTTTGACTCTTCAGGGACACCTGGAGCACAGCGCCCAGTGCCTTCTACCTCCCACCC
CTTACAGCTCAGGCAGGTCCCAGGAGGCTGACCCATAGCCTTTTGCTCTATCTGGGCCTGTGATATCTGGCTGGCATCT
TTGAAAGAGCCCACTGTGCCATTCTTCCATTTATCCTTGTCTTGTATGTCTCTGCTCTCATCCAAACTCCTTCCTCTCC
CTCAAATGTGCACATGAACACACAAGTACACATAAATGAATGTCCACTTGTGCATACACACCCACACAGCAGATTCTTG
ACTCTTTACAAGTAATGAACTTTGGAAAGTACTGCTTTATCATATATAAAAGAAAAAAGTTGTTTTCTTGCAAATGTTG
CAGGGCCCTGGAGGGGGGGGAAGGTAGGAGGGGGGAGGTTAGATGTTCAAGAGGAGAACAAAACAAAACCACACTCCAG
GAGCTCTTAAAAGAAGATTCTTTAAGAAAGAGCTGCAGAAGTAGCAGCCTCACAAGAGCTGACTCAAGACTGCCTGGGC
CTGGAAGACGGCCACACATCTCCACGGCCCTCGTCTGCAGCATCCATCCATGCACGGACAAGCACTCTGGGACTCTTGG
TGATGGTAGACAAAGAGAAGAAGCAAGTATGTGGGTAGAGCCCCTGCTCAGAGATTCTAGGGTGATGCTTCTGTACCTC
AGGCCAAGGTTCAGAGAATAACAGTGAGTTTACTGAGCAGGCTGTGGCCTCAGAGACCTTGCTCCTGTGGTCAGGATGT
TGTAAATAAGGAGATGCCTCCTTCCACACCCGCTCCCATCCCAACCATGGCAGCCACTTCTGTTCTTGTGTTCTTGGAG
GAGAAGAATTCACACCAGGCACAAGCAAAGCAAGCAAATGGTCAAGGTTTCTTTTAGCAGGTGAGACTGCAGAGGCAAA
GTACACCGCTATAGTGGAGGGCTGACCAACCCGACTCTTAGAGGACTCAGCCTACTGTTCAGTTCCCTTTGCCCACCCT
TGGGACCATCCCGTTCCAGGTAAGGGCAGAGAGTTTCTTGCAAGCCCTCTTGCACACAATTACCTACAGTTATTCTGCT
TCTACTAGATACAATAAGTGAACGTATTTGTATAAAATTGTAACTACCTATGGAGTTGACAAGTTTACGAAAGGGAAAG
AGTACTTTGTGCATGCTCTGCGCATGCTCCCCTGGCGCTTCCTGGGCGCCTATTCAGAGCCAGTGGAGACCTGTTTTAT
TGTTTTTCCCAGCTCCCTTTCTTCCTACTTAGTCATCTTGGCTATTGCATTCTAAGTTGAAAGCCCAGGTATTACCTTC
CTCAGTGTTTCCAACTTCGTTGTTTTACAATTAATATAGTTTAAAGCCTAATATTTGTTTTCATTGTAGCAAAGAGGAA
AGCAGACTAAGATCTGTAAGGGCAGGGCGCCTGCGCAGCAGTCCACAGTAGGTTTCTGAGCCAGCAGGCAGCATTTGGG
TGTATCGTGGTGTCCAGGGGTGACATCCTTGCCCATCTATGGAGCTGTGATCAGTGCAGTGAAGTCCCCACCATGAAAC
CTTGCAATGACAGAAAAGCCCTCTCAGAGACCCGGAGACTGGAAGACTTGATTTCTGCAGTGGTCCAAGAAGACAGTGG
GACCAGTAGGCAAGGCTAGCCTCTAAGTGAAGACAGCTGCCTGTGTGCCAATGGATATCTGTGTGCCAATGGATATGGC
AACACTCGTGCCAGACACCCTTGCCCTCTACCCTAGAGCCTGAGCATGCTAACTAACACTCTAGCCCTGAGGTGTGACT
ACAGGCACACCCGCAGTGAGTGGCAATCAATGGGGGCCACTTCCGACATGATGCTAAGAGAGAGAGGGTTCTGATCTCA
CCACCAGACCCCTTGAAGTCAGTCCAAGTGTCTGGGCAAGAAAATGAAACATCCCAGAAAGCGAGTGGAAGGCAGCTTT
CCCTGCAGGCCTCTTGTGCTGTTTCTCTGAGTTCTTCCGTGATGACAGTTCTAAGAGTGTGGGCAGAACTGGGCAGTCA
CTTAATCCAGCCTGTGGTGAAAGAATCATTCCGCTGCAGAGGTTCCTGCCTCCTGGGAATGTATGAGTCCTTAGCACAG
CCAGACAACAGCCATAAATGAAGCCATGTTTGTTTACAGCCTGTCATCTTCTCTACATAAACTTTCTATGCACTAAAGA
AACAACATCTTCCATTGATCACGAATGAAGTTTGCCAAAAGAAATCTGGGAAGCAGCACGCGATGGAGATTTTAAAATC
TATTTCCATCCGTTAACTGACAGAAGAAATCTAGAAAGAATTAATTCACCAAAGGCTTTTTGGACAGCCCTGCTAAGCC
AGTGGGGGAGCCCGTGCCCTTCTGGTCCTCAGCCTTGTTTATTTCCCTCCAGGCCCAGCCCTGGTCTTCGTGTCTATAA
TATGACTCTGAAAATCCATTTAGCCCAGGTGAACTCCAGATAGCAAGACAAAACAGAGCAAAGGGCAAAAAACACCTGT
GTTCTGTGGAACCATTCTGGTTTGTTTGTTGTGGTCCTGGGGACTGAACCTGGGGCAGCATGCATGGTCTGCAGCAAGT
GCTTGACTACTGAGCTAAACCATTATTCCTATGCACTGTTATTTTATTTTTCAAGTGTGCGCGCGTGTGTGTGTGTGTG
TGCGTGCGTGTATGTGTGCAAGTGTGTGTGTGTGTTGTTATGATGGTTAGTTTTAACTGCCAACTTGACATGAGCAAGA
ATCGTGTGAGAATAGGATCTTAATGAAGCACAGTCTAGGTCTTATACTGGCCTGTGGGTGTGTCTTGGTTGTACATTGG
TGTGGGAAGACCCGCCGTGAATGTGCGTGGCACCATTTCAAGGGCTGGGCCCTGGACTCTATGAAGGGCAATGAAAGCT
GAAAGCCTTTACACAGGCAGCAGGGGAGCACTAGTCTTTCTCACAGCTCCAGACTGTGGGTGTGGCAAGTTCCTACCTT
AACCTGCCTTAATAACGGACTGTAGCCCAGGGTTGTAACTCACATATACCCTCCTGCCCTCTGGTGCTTTTTAGAGAAA
ATATTTAATCGCAGCAATAGAATGCAATTAAAACAATTAGGGTATTCCTGTATAGGGTATATGCATTCGTGTGTGTGTG
TGTGTGTGTGTGTGTGTGTGTGTGCACTCTCTTGCACACTCAGAGGCCAGAGAAGGATGTGGGATATCTTGCTCTATCA
CCTTATACCTTGTTTCTTTGGCACTAGAGCTTCACTGACCCTTGAGTTAATCTGGTGGTGGTGCCCAGCAAGCTTCAGT
GACCCTCCTGACTATGTCCTCCACAGAACTGAAGTTCAAACACACACAGCTTTTAACATGGGTGCTGGAAGTAGAGTTC
TGAGTCCTCTTTCTTATACAACAAACACTTACCCACTGAGCCTCCTCTCTAGCCTCCTTTCTATCCTTTTATAAATGAG
TGGTCCTTAAAATCCTAACTCAACAAATTTCTCGAACATCTTAGTGCTCAAAACTATGTCACATGACCATCTAATCCAT
ACAAGGCCTGGAGATATTGTTTTTCCTGGTCACTTTTGGCACAGCAAAATGAGATTTCTGTGACTTTGGGTGAGGAGAA
AGGACACAGTGGGCAGCTGGCTGTCTCTACCAGGTGTCTCCATAACAGCCCTTTGTTTGTCGGAGGGTCTGGGAAATGG
ATCAACAGGAAAAGAGAATGGGGACCAGAGGTATAGAAGAGACGGGAGAGATTAGTGTCCGGATGCCCAGCTCAGCTGA
GAGCAGGCATGCCTCTTGTGATGACCAGCAGTCAGTTTGCTGAGGATGCAGGGCTTTCAGTGCTAAAACTCAGGCAGGA
GATGCGCTGTGGCTCAGTGGAAGCCTGGTGTGTGTGAGGCTCAAGGTTCAGACCTCAGTACTTCAAGAAAACCAAGGCA
GTTCCAAGCAAACCTGGGAGGTAGTCTACTCTAAATGTTTACAGGCCAGCATGAGGCAGTATTAAGCAAGCTGCCCTTG
ACCAGTGCTCCCCAAAGCCCTCCACAGGTCCCCAAAGCCCTCCATTCTAGAGGCTGTAAGGTCAGAAGAACTTTCCAAT
GACCTGATTTATTCATGCAATTCACACTTCTCTTGCACTGTTACACAAAGGAGTTCCCCAGAGATTCTATGGCCTGGAA
GCAGTCAGCAGACAGGGTCCACAGGTGGAAACGGGATCCAGCTGCCTTCAGCTCCTCAGGGAAAAGGAAGGGAAAGGTA
TAACCTAAAGAAAGCCTGGAGTCGCAGACACTTGTGAGCCATCATATACAGGTTCTGGGAATCAAACCCTGGTCCCCTG
GAAGCCCAAGCACTGTTCTAATCCAATGAGCCATTTCTCCAGTCCCTTTACATTGAATGTTTTAAAGCCCTTTGATGTT
ATCAACAAATTGTAAATGTTTAAAAGGAAGCTGAGAACAGAGATCCACTGTCCTAAACTAGAATAGGGATGGGGGGGGG
GGGCAGTTAAAGTATAGAATGTTCTGGAAATTAAAGGATAGAACTAAAACCAAGAATAATACACAGATCCAGGCATCAG
GAAGTACTTCCAGGACCCTCGACAGTAGTGCATGTGTGTTGGTGCAGCAGCGGGGTAGGGAGGTGGTTGTGGGAGCATG
GATATACCTCTGTGTGGAGCTGTGAGTTTTCTGTGTGTGTCAATGGCTTATTGCTGTACAACTTAAAGTGTGGTTCAAA
AACCAGCTCCCAAGGGAACTCTTCAACATCAGTTACGGTAGACGTGAGTACAGAAATGGGGGCAACTTGGAATACCCAA
GATTCAATTCACAGACCATTTGAAAAAATAGGAGAAGCTTTAGAAACTCCTACGGTAGCTCCACAAAGCAACTTGACAT
TCGTCAAGCTGAAATCAAGAATCTGGGCTTATATATCGTTTATATTTGCTGAACTTTTTTTCCCTAGAAATTTCTCTTC
ACAAAAGTATCCCATGATGCTTTGAGAAGTTGAAAAAATATGGTCATGTCATACTTGAGATGAACTATACAGGATAAGT
ACTAACTATTCAAAGAGGAGGGATATAGAGTGATATGATGGAAAATGTGTGCTTTGGGGAAGACTCTGCACGGTCTGTG
ACACACTCCTCCTTATCACCTGATCCTAGCCTTCTCCCTCTGTGGGGACGTCCAGCTCCCCCTCCATCCGTTCCCTACA
TCAAGATTCCTGGTCTTGAGATATATATATATGTGTGTGTGTGTGTGTGTGTGTGTGTGTGAGAGAGAGAGAGAGAGAG
AGAGAGAGAGAGAGAGAGGGATTTGAATTCTCTTCAGACCCAATAACAAAATCTTACAAATTTCTCCAACCATGTATTT
TACTTAAAATCATTAATTTGCATTCATTAAAAACAAAACATTATTTTCCCTTATTGAAAAATTGTTACCCAGTTTATGA
AACGCTCCTTTTGGATCCCACCATTGATGTAACTCAGTTGCTCTCTGGAATTCTTCTCTCTCTCTCTCTCTCTCTCTCT
CTCTCTCTCTCTCTCTCTCTCACTTGCTCACTCGCTCTCTCTCTCTCTCTCTCTCTCTCTCTCTCACTTGCTCACTCGC
TCTCTCTCTCTCTCTCTCTCTCTCTCTCTCTCTCAACATCTATTCACTCCACTCCATACATTTTTTTCTTCTATCTTAG
CTTCTCTTGCATGCTATATTGGCATGAATTAAAAGAAAGAAGTCATAATAAGCCAAAGGAAAGATACTTGAGGTTGGTA
GGAGGAAGAAAGAGGGCTAGGGAGTGATACAAAGAAAAAAAGAGAGAAGAGGGATGGGTGTTGAATACAAGGGAACCAA
GGGATCATTCCATGACGTCTCTCATACATGAACTACAGATACTTGTGGTCCAAGATTTCATAGCTATTTATTGTTGTAG
GTGTTCAAGACAGGCTAATTGTACTGGGCTTAGAGTACTAACAGCCAACAGCTAAAGTTACTGTCCTGAGCTGTGGTTG
TCAGGATCCTCAGCTCTTCAAGTCTTGGCAGCTCTCTCAAGTTCTTCTTGTGTTCAGAGCTTTTCAGTCCTTGTAGTTT
GGGCTCCCACCCTCAGCTCTTGACAGCCACTCATCTTCCCCAATCCCAGTTTCTCTGCCATCCTTCATAGTCTTAACAC
CTCAGATTCTGCCCTGGGTTGCTCTCTGGAACTCTTCGGCTACCTACTAATGTTACAGCTCAGAGCTTTGGGTATTAGG
GCCCTTTGCTTTAGTGGCTTTTTAGAACTCTTAGCTACCTCACCCCTACCACTGTCACTGCCAAACCTAAAGACTCAGG
GTCTTGTCTGCTTGGTGTAATAAGCCCAACCTTCTCTACTTGTGACAAAGTACATAGCCAGGCAAGAAAAAAAGCTTTT
ATTGGATCATATTGTCATAACACTGGAGCATAGTACCCAGGAAATATATCTTAAAAAAAAATAAAGTGACAAACAGCCA
CCTCATATAGCCATAAGGGGGATGAGGTATGCTTGTAGCTAGATTATAATTCCACCAGTCTTAGCCTTTTATATGCATG
GCTCCCAAGTTCACCAATTATAAGCTTTATTTACCTAAAGAACAATTACAAGCCAAGGACCAATTGCAGGCTTTTTAGC
TGCAAGGGAAGCAGTGTCATCTTGAGTCCACAGATATGTCTTGTCCAAAGTGGAGTCTTCCAAAGCAAATGCAAGCTGC
ATGGGACTGTCATGACTGGAAAAAAGGCCTCCTAAAGTATAGGAGTCATAGCTTTAACAACAGCTATAGTTCCAGTAGT
CTACAATTGGAGTGCAGCTTCCTCAGATTAAATATGAAGTGTTCAGAGTGCCAAAAGAGGTTTGCTTGTCTGACAGATC
CCCATTGAAGGTTGTTAGACACAAGTTAAAGGTTTATCAGCTATCATTCAAGAAGCTTAACAGGTAGTCCTAACAGCTT
AGCTTATTTGGGGGATGGACATTAAAGATGCTTACAATAAAGACCTATCACAGAATTCTCAACAGAACCTAGCAGTGCT
TAATATCTAGTCATCTAGCCGTGGGCCCTTAAGAAGAATTGGATTCACTTTTATCTATCTCATTAAGGACACGTGAAGT
TGGGATGTCTAAGGCTCTGCCAATGTTATTCCTCTTACTCTCAATATCTACAACACCTTTCCCACAGCTGGTCTGGGTC
CATTGCCTTATCAATACAGGCTCTCTTTACTATCAGCCCACTTCTAAAACCTCTTCAGCTCTTCTATAGTCTCCTTAGT
TTTTGTATATTCCAGTAACTTACATAGATCTGTATATAGAGATATATTTAATGCATGATGTGAGAGTTAATAATTAACA
TTCAAAAAGAAGAGTCTACATTGCCTCAACCAGATTACCAAGGTACCCAGCAATGTGCTGCTACTGAAAATACAGCACC
TTATAGATTTATTATTATTCAATAAATTATGACTGTGGGAAGACACAAATATATTTACCTGCATTTCTGGCAGTGTATT
CTCTATGGTACTCAGTTGCTACAAACTCATTCTCAAACCCAGAGAAACTGACATACACACGCATGCAGCACCATGCTCT
TTCAGAAACAGAGTGTGACACAATAAATGCATACAGAAGTCAGTTCACTTCTTGGCTTGCTGTAGTCTGAGCTGAGTTT
AAATTAGAGTGAGTTCACATGTTTTCCAGGGGGTCACCTAAAGTGGCTTTGAGAATTTAAGTGAATAAAGAGGCACATT
TACAGGGTACCAGCAAGCATGTTCCCCTTTGCCTTTTGAGAATTTATCCAGGCTGCATATTTTTCTGGGCCACTAAACT
TGTGCAACAGAAGTACAAAGAGCTGTGTGGCCTAGAGTAAACCATTTAACCTCTCTGATCTAAAATCTGAGAGGAGCCC
CTCATCCCGAAATTAGTTAGAGCCTTTTGCCAGCAGAGAAGGAAGAATCCCACACAACAAATAACGGGGCCTTTCTCCA
CAGCTGCTCACCAGCAATATTCTAAGAATCTCACTAGAAAACCCAATTACAGGGATGGGCATGTATTAAACCCCTTGTG
CAACCCTATACCAAATGATAGAGCACTTCTGGACAGATAATTTTACTCATTAAAGGAGAAAGAAAGGATTTGGGGAGTT
ACTTAGCAACTTTTTTAGCTGTAACGTGTGAGCTATCAAAAAGCATGTATGGCTAATGCTAAGCTGTTGTGACTTCCTC
ATGAGGAGTCGGTGCCCAGGCCTTATCTATCTCAGTTTCCACAACTCCCCTTGGGGAGGACACGCTCATATCCACCCTC
TGGGTTGTGCCTTCACCTCTTCCCAGCAGCTCTTTTTCTCTTAACCCTCATGATTACACTTATGCTAAAGTATCTTTAA
TGATGGGGTGGAGAGATGGCTCGTTGGTGAGCAACATCTGCAGTTTGCTGAAGACCCAGGTTCGGTTCTCAGCATCCAT
TTGGTGCCACACAGCCGTCTGCAACTCCAGTCCCCAGGAATAATCATATGCTTCCTCTGGCTTCTGCATGACCCTGTCT
GCATGTGACACACATGCATGCAGGCAAAACATTCGTAGGCATAAATAAAAGAAGTGCCATCCTCAAAGGTAGATCTTTA
ATTAAATCCCCAACTCGTTCACTGTACCAGTTAGTTGTGAAATTCTTTTCGGTATCAAAGCCATAAACCTCAGCCTGGT
CTGAGTCAAGTTCCCTAAAAATATATGGGACCTTTTCAAGCTGCTGTGGGTGACAGTGTGGAACTATGTCTGGGTCCCC
TCTCTGCTGGCAACAGACTGTTACAAAGCTCGGGGGTGGGGGCGGCGCGGGGGAGAGGGGCGACACGATGACCCCTTTC
CCAACGTGTGTCATTACTGACATGTATCATTGCTCTTAAGTCTGCTGAAGAGACATTAGATGGCTTTCTCCCCTTCCAT
CACTGACTCCAGGGCGGGGGTGAGCGGGTGGTGGTGGGAAGGCTCTGCAGCATCATGGCTGAATGAATGGGTACGCACT
CAGAGCTGTGTTTAGCACAAACCATACCATGTTTGCAGCATGTGGTCAGGCAAATTAACCATTTTCTTTCACTAACATG
GTGTGTCTCTGGCATTGCTTCATTCCATAGGAGTATGGTAAGGCTCACATGAGACAGTGAGCAGAGAGTCATCCCTATC
CTGTACCTTTAAAACAAACCTTTAGTAATACCTGCAGTTACTTGTTCATAGCTGACGCCATTCGCTCAGGCCAGAGGCT
ATCCTCATGGCTACCGCAGAGTCGGCTAGGTGAAGCCTTGTGTGCGGTCTGGTGTGGGTATTGATGGAGTCAGGGTGAG
ATAAGGGAACCTTGAAGGCCCAGAACAGACCCGCATTCCCTGCCAGCTAGACGATCTTGGAAGATTAAGACCATGTCTG
CCAAGGACACTGCTGACCCCAGCCCGGGTCAGAAGCGCGTTCCCACTTTGGAGCTTTCCAGCCTGTCCCGGCACAGAAC
AACAGGTGTGTGTATGTGCTCAGTAACTTGAACCTATCTTTGGAGCCTTCTAAAATTGAGAGAGGACAGACAGCTCTTT
CTTCAGACAACCACAAGACCAATAAACAACTTTTCAGGGAGATCGGGGCCAACTCCAGACTCAGCAGGGATGGCTGTCC
TTTTAGGGACTCTTGAACCTGTATTTTCAGTAGAGCGCCATGGCAGCAGTACCACCGCCATTCCTGGCTGGCTGCTGAA
CCAGGTTCTTCTTTACTGCTCTTACAGACCCAGGAGCAGAGTGGTTGCTCAGAGCTCTAGCAGATGGGTCCTGGCAGGA
GCCTTAGCCCTACCACAGGCTTCTTGCTGACCTTGAAGAAGCTGGCTAACCTAGGGCTTTTGAGAAGTTTGTGGTAGAG
CTACAGTAACATTAGCAGCCCAGAACTTGGCACAGAGTGAGTCTCAGCGCATGACCGAGCACTGTCCCTCCACGACCCG
TGGGGACACTGAAGCCCACTCTGGCCAAAATCATGCTTTAATCTATCACACAATCCCCAGCACAAGCCTGGCCTGTTGT
CCTGCCTTTCTGTGGAAGCCCTAGGGTGGTGATTCTAAACCTTGCTAATGCTGTGCCCCTTTAATACAGTTCCTCATGT
TGTGGTGACCCCCAACCATCAAATGATTTCCTTGCTACTTCATAACTGTTATTTTATTACTGCTGTGAATTGTAATGTA
AATAGCTGATAGTTAGGGTATCTGATTTGTGACACCTGTGGGGTCACAAGTCACAGGTTGAGATCCTCCATCCTAAAGT
GAGACACTTGTGTCCCTCTCTCTGCTCACCAGCCGTGCTAAACAATGTACCTCAAAGAATGGAAATCTCCGGGGTTGTG
GGCGGGGCTGTGGGTGGGGCGGGGGTTTGGGGAATCTTGCCATTTCCCTCCTAGGAAGAGATTGCCCATTACTCTGGAA
ACTTCTTTCATGATTAAATTAACATATGATATTGCACATGCCAGCAAGAGTTTGCTGAAGGGACCCTGATATAGCTGTC
TCGTATGAGGCTATGCCGGTGCCTGGCAAACACAGAAGTGGATGCTCACAGTCATCCATAGGATCGAACACAGGGCCCC
CAATGGAGGAGCTAGAGAAAACACCCAAGGAGCTGAAGGAGTCTGCAACCCTATAGGTGGAACAACAATATGAACTAAC
CAGTACCCCCGGAGCTCTTGTCTCTGGCTGCATATGTAGCAGAAGATGGCCTAGTCGGCCATCATTGGGAAGAGAGGCC
CCTTGGTCTTGCAAACTTTATATGCCGCAGAACAGGGGAACACCAGGGCCAAGAAGCAGGAGTGGGTGGGTAGGGGAGC
AGGGCAGGGGGAGGGTATAGGGAACTTTCAAGATAGCATTTAAAATGTAAATAAAGAAAATATCTAATAAAAAATTTTA
AAATAATAATATCAAAAATTAGCATATGCACATTCCCTGGCTTTTGTTTTGTCTACTCTACAATATTATAACACTGCTA
CCAAAAAAAAAAAAAAAAAAGAAAAAGAAAGAAAGAAAGAAAGCCAAAAGAAAGCTAGATATCATAGTGCCCAGATTGG
CCAACCTGTAACTAAATCCATTTTCCGTTACTGACTTCTGTTCTCTAATATTTATGCTGCGGGTTTCTGAGCCTATGGG
CCCTATAACAGGAAAGTCTGGGGCAGAAACAAGAGAGTAACCCTCTGCTCCAATGATCTACCTAGGACATGGAAGGCAC
TGGCTAAGACAGGGCCACAGCACCCCTCAGCCAGGCTCTTCCCCGCAGCTCTACCAAACCAGAACGTTCTTTCCACGTA
GCCTAGGGTGACCTCAACTTTCTGACCCTCCATCCTATACCTCCACTGTGCTGAGATTACAGGAGCACACCACCAGACT
TGTTTTTCACAGTGCTGTGATCAAACCAGCAAGTATTTTACCAACTGAGCCGCATCCTCAGCCCAGGGACATTCTTCAG
AAAGGGCTAAAGAGCTAAAGACGACCCTCCAGAGATGCAGCCTCGTGTTATCTTACTCCAAGCACAGATCAGAATCAAT
ATTCAGAGTCAGAGACCACATGTCTGTGCCCAGACTTTGAGATAAAGGGTAAAGGTTAAAAATAGATGAGAATACACAC
ACACACACACACACACAAACACACACACACACACACACAGCTATGAAAAACACCCAACAGAGGCTGCTCAGCAGTGAAT
CACTCTCTAGAATCTTGTACACTACCATGTTGCTACCCACAGCTATATGTGTGTTCTGATGATCACAGTTCCCCAAACA
AAACCTTAATGATGTCACTCGTTACTTCTTCTTGGCATACTTTAAGTTGGTATAAACACATTTATAAATAAGCTGTTTG
GCATGAAGCTATGAATTTTCTCTACTCGGGAGATTTTATACGTAATGTTTATAGTATGATTTTGACTCACAAAGGTCTA
CTTAACCCACAAGATGTTGGTAAGTCTGTCTTTTAGGGAAAAACACTTACATTAGAACACATAAAACATATACAACCCA
ATTAAACATAGTCTAAGGTCTACCCAGCGTCTGATTCCCCTCCCCTTACTCAGATCATGTGGCCTCAGAGACCAGTCCT
AGACCCAGGGAAGGTCATGATTAGCAGGCCCATAGTGATTAGCCCGTCTATTGTCAGTGCCATGCTAAGGGATAATGTC
ACCTGGTGTTCCTCTGAACTCTTGAGGTCCCTGAACAGAGGAGACCTGAGGTGGTTCAGTTAGATCAAAGACCATCCCC
GAGCATCTGTGAGTGTACCAAAGGACTCTTTATTGTCTGGCTAAAAATTAACATGGATACATGCTGTCATTGTTGCTGC
CAGAGGCCATCTTGCGTGCTCAGCTGAGGTCTGCCTGAAGATAAGGTACTGCAAGTGATCAAAAAGGTGGGGAAACCAG
TCTTGGCTTCACTGAGCCACACCCAGCTTGTTCTGTGCCACCCACCCCCACCCCCATCCCCAACCCCGACTTCTTGTTT
TACAAATCGTATGTTAAATGTGAGGGAGTTTCCTAGCATTCTAAGAAGAAAGAATCCATTTTGCTGGCAGGACCTAAGC
CTGAAGATGAATGGAAGAGAAAGAACTCTTGAGATAACAGCCAAGCACTGGCGTGCATAATCCCAGCACTAAATTGAGG
GGAGGGGGGAATGGCTAAGTTGGTGGCCAGCGTGGACATGTGCAGTAGAAATCTATCTCAAAAACAGTTGACCAGGTAC
TAGAAGAAAGAAGCAGAGAAGGAGCCCATGCTTAAGCAGTAAGGACATTTCCCCTCGTGAATCCCAGAAACCATGACAA
GGAAAGTCGGATTTGAACACCAGAGAAAGAAGAGATGAGACAGTGTTCCTAACTCCTGAATCATATACTCAGCCCAGAG
CATCATGGTCCCCATTGTTCAAGAACAAAAGCAAAATGACAGGGCCGTGGACGACAGGGGAGAAGATGCCCCAGGGACT
ACCATGGTCCCAGAGAGCTCATGAGTCCCTGGAAGTATGTATTTTCTGGAGCTAAAGTCCTGGTAGGCAGCTTGCTTCC
TCTCCCAGGAACTTCCTTACAACCTCTGATAGACATGAAGATTTGCTGACTCTCCCCTCCAGCCAATAGATGTTCTTCA
CACTTCCCCCTAGACTCAGTGAACACAGATCTCCTTCCATAACAAAATCCAAATGAGAAAGGGAAATGCCAGACCCATC
TGTGATGGTGAAACCTCATAGCGCACATCTGTGGTCTGCCTGCCACAGCCTGCTCCTGCTCCTGCTCCTGCATCTGCTC
CTGCTCCTGCTCCTGCTCCTGCTCCTGCTCCTGCTCCTGCTCTTGCTCCTGCTCCTGCTGCTCCTCCTCCTGCTCCTGC
TCCTGCTCCTGCTCCTGCTCCTGCTCCTGCTCCTGCTCCTGCTCCTGCTCCTCCTCCTGCTCCTCCTCCTGCTCCTCCT
CCTGCTCCTGCTCCTGCTCCTGCTCCTGCTCCTGCTCCTGCTCCTGCTCCTTCTGTTAGCAGCACCCATCATGCCTGAG
ACAATGTGGACGACAAAGGATGAAACAGGACCCCCGATCCACAGATGAGCATGTGGCTCAGGTCTAACCAGTGTATCTC
CCTCATTGTTTCAGATATAGGCAGATGAACCCCAACCACTAGCAAAGAGGAGGAATTGATATTGAGAGGGAGACTGGAT
TCTTTATCTTTACCCAGGAAGACATGAGGTTTTGGGCAGTTGCTAGGACCAGGTTGGTAATCTATGAGAAATGAAAAGC
CTTTCTGGCAAGGAAGCTAAGTCAGAGAGATGCAGGCAGAGCGAGAGACAAACTGCATCCTGACTACATCATATGAGCC
CCTTGTCTGAAGCCAGCACCCTACATCTTACACTTAGATGAGTGTGTGTGTGTGTGTGTGTGTGTGTATCTGTCTGTCT
ATGTGTGTGTATCTGTCTGTCTGTGTGTGTGTGTGTCTGTCTGTGTGTCTGTAATTTTCACATGGTTGATATGGTTGGT
TTTTCTATCACTTGTAACTGATAGAGTCTGGGCTAATAACACTTCTCAACCTTAAAAAAGAATGGCATCCCCTCTCTTA
CAATTCTGGGTTTTATGTAAGAGACCTAGGTTGCTCCTAATGGGCATAACTGCCTGACCTTGAACAAGTCACAACCAAG
TGTTCTCAGCAGCAAATGAGGGTGTTCAATCTCCCAGGGTTTTTTTTCTCCTGTTCTGAAATTGTGTATAACTATGAAT
AGTCTCTTAGCAACCTTCACATTCACAGAATTAGATGCAAGATGACTTTCTTCAAGTATATGGATTTAAAAATATTAAA
ATATAATTTCAATACTTTAATTACATGCCAGATTTTATAGACTATTGAAGGCCTCTTTGCTTTAGCAAGGTACATAATA
ACACTACATTCATGACACACATGTGTCATCTCTAATTTCTTATTCTTTTAAAGATTTTGTTTTTATTTTTAATTATGGG
GAGGGTTGTGCACATGAGGTTGGTACCCATGGTGGCCAAGAGAGGGTATCAGATTCCCAGGAACTGGAGTTACAGGCAA
TTATGAACCACCTGATGTGGGTACTGGGAACTGAACTCATGTCTTCTGCAAGATGCACTCTTAAACAGTCAAGCATCTC
TCCTGGTCCTTTTCTCTCTCCAGCACCTGAGAACATCCACTACCCTTGCCACTTCTGATGAAGAACACCCAGTGTATCT
TTGCTAATCTATCTTATGGTTAACCTGTAAGCTTTTATATAACATAGGTTATATCAGTACACAGCTATGGTGTCCCAGA
TAGAAAGTTAACAAGTCAGAAGAGACAAACTTCAGGCTCAGCTCACCTGACTACATCTCTGGCTACTTCCTAGTCCAAA
ACTAACCTCTTCTTTTGAGAGGTTTTGCTGTCAGCCAAGAGGGCTGGGGTGTCCAGCTAATGTGACTTGCAGCTCTTAG
TGTGAATTTGCTCTTCCTGGCATCTAATTTGAACTCCTCTCAATACCACCGCAGGTTCATTTCTTGGTGCTCTGTTTTA
GTTGAAAAGCCCTCAGAGCGGATTCAGTGACTCAGAACTCAATTCTCTGGGGGCACTCGGAGGCGGCAGAGCTAGAGAA
TGGATTCTTCTGGCCTGCTGTTAAGTGAAACCAACTGATGCCTTGCCTGACCCATTTATCAGGTGTAATGGGCTGGAGC
ATGCTCTGAAGAACTCGCTCACGGAGGCTGAGCCACTTAAACCATGCTCTCAGAGCACAGGATGCTCTCTGGTTGAAGA
ACAGGAGCAAGAGGTCAGGTGAGCCTCTAAATTCCTTGAAGAGTGTGCCCATGGCAGTTGTGAGCTCTGGCATGGCACG
ACCATGCTCCTGGAAGATCTGCTCGTGCTGACTGGCTCTAGACACTTTTCTTATTTCAAGTCTTGGTTTCTTCTCATGT
ACGGTAGGGCCTCAAAATCTGGAGTAACTGATGTCATGTGTGTAATACATCTGCTACATAGTCAAATCATAACAGTTAA
ACCAATCAAGAGAAAGTGCTGTGCTGGCCAGAAACAACAACAGTCCCAGGTGCCTTACTGTCCTCACCCTACCAATAGG
ATGGGTAAACTCCAAGGAGTTATCACACTAGACTCTGCTGCCTCCCACGACAGCTTTGTCACAGTTCCATCTCCAGGGG
AACTCAGCAGTCTCTCCTTGATTATTTTACCCCATGTTGGGGCTCACTTACATTATTGGGGTCAGATTTATCAAGCTTG
TAAGAGAGAGCAACACATTTCCTTACTATTTTTTATGGGGTTCATGGATTCTCAGCTAAGGTAGGATATTAGTTAAGAT
GTCCTACCTTAGCTGACCTTCAGCATCTCCTGGCCAACCCATCGGGCCAGTGTTCCTGTGGCTCACATGGTCATGGATC
ACCTGAAGGCATGGGATATCAGTCCCTCACTTCACAGTTTACCAAGTGTCACGGAGCAAAGTGACAAGCTTTCTCTTCA
GCAAAAGTGGAAGCCAGTTCCGTCACCCCCACCCCCACCCCCACCCCCGCTGACTCCTTTTTATGGCTTCTCCCTCCAA
AGACAGGCTTTCTTGGCTCTCTCTCAAAATTGGTGACCCCTCACAGAACAGGAGGTCCTGAAGAGGGTCTACTCCCCGC
ATCTTGGGTCCTTTCCAGGCTTTGAGACATGTCCTGCTCCAGGCCCGCCCCTACATGTCCTGAGTGCCACCTGTCCCGT
GACTTCACTAACAAGTGCCACTGTCCTGTGTAAATCTCCTCTGGGCTCAACCTCCAGGGCAAGAGAGGAGAATTAGGAC
ACCAGAGTTGAAAGATGCTGGCTTCAGAAGAGAGCGCTAAGCAGACCTGAATAGCTGAGTGTTTCAAAGCACCCGTGGA
AGTCCGTGTAGAACTGCAACATCTCCTCCAACCACTGGCAGTTCCCGGCCCTCCTGGATTCCTTTGAGTAGTAGCTCTG
AATTCTGCATCCTACAAACGCACCTTCTGCATTTCAATGGGGAAAGATTGGGGACAGCTTTGGGCCTTCAAACCATATG
TTTGCCTCTCCAAATCATGAAGCCAGAGATTCCCCTTGGGTAATAATAGCAGTTTTGAGCTGGAAGACTTGAAGTCATG
TGATGTTCAGGAATGTAAGGTTTGGTAAAGTCCACTTCTTTTCCCAGACCCCATGTAGGGGTGAAAGCCAAGCTGACTT
ACCCACAAAGACTCAGAGAAACAGTCCTTGCCAGAAATGCTCAGACGGCCCACAGTTATTCCTCCTCCAAGAGTACACA
CAGGTCTACAGCTCAAAGAATAAATTCACTCTACCATGACACACCCATTCCTCAATGGCTGCTTCTTGCATGGACTTGA
CAATGTAAATGGCCCACTCTCATGACCTTACATTGACTTAATACTGTAAAGATTATTTCCAAATAAAGTCATATTCTGA
GACATTAGAGGCTTAGGAGTTCAAAATATGAACTTAAAGGCGGGGGCATTCCAGCCCCCAACAGTATGTATATTCATAA
AGAAGAAAAAGCAAAACTGTCTTGGTGAGATAGAACAGCAGTATGGCCTGTGAGCAATTTTTCTTAACTGTGTGGGAGC
AGCCTGTCACACCATGGTAAGGACAGCACTCCTGCTACAGGAGGCTTCTAAGCACAAGAATTCTTCTGTTCTCCACCCA
CCCCCTCTGCTGAAGGAGGTTCTGCTGGGGTCTGTCTGTGACCACATGGTGTTTAAACACATCAAGCCAATATAGGTAT
CCACAGACAAGCCTGGCTCCTAATGAGCTATGGGGTAACTTCCTGCTGTCAACATTCAGTTTTCTCATCTATAAAATGA
AGCTAGCCTGTTCCAAACACTTCTCAGAGAAGGGGAAATAGAATATAGGACAAGACAAGTCCCTTCCGCTCGACTCGAG
ACTCGAGCCCCGGGCTACCTTGCCAGCAGAGTGTTGCCCAACACCCGCAAGGGCCCACACGGGACTCCCCACGGGACCC
TAAGACCTCTGGTGAGTGGACCACAGTGCCTGCCCCAATCCAATCGCGCGGAACTTGAGACTGCGGTACATAGGGAAGC
AGGCTACCCGGGCCTGATCTGGGGCACAAGTCCCTTCCGCTCGACTCGTGACTCGAGCCCCGGGCTACCTTGCCAGCAG
AGTCTTGCCCAACACCCGCAAGGGTCCACACGGGACTCCCCACGGGACCCTAAGACCTCTGGTGAGTGGACCACAGTGC
CTGCCCCAATCCAATCGCGCGGAACTTGAGACTGCGGTACATAGGGAAGCAGGCTACCCGGGCCTGATCTGGGGCACAA
GTCCCTTCCGCTCGACTCGAGACTCGAGCCCCGGGCTACCTTGCCAGCAGAGTGTTGCCCAACACCCGCAAGGGCCCAC
ACGGGACTCCCCACGGGACCCTAAGACCTCTGGTGAGTGGAACACAGCGCCTACCCCAATCCAATCGCGTGGAACTTGA
GACTGCGGTACATAGGGAAGCAGGCTACCCGGGCTTGATCTGGGGCACAAACCCCTTCCACTCCACTCGAGCCCCGGCT
ACCTTGCCAGCTGAGTCGCCTGACACCCGCAAGGGCCCACACAGGATTCCACACGTGATCCTAAGACCTCTAGTGAGTG
GAACACAACTTCTGCCAGGAGTCTGGTTCGAACACCAGATATCTGGGTACCTGCCTTGCAAGAAGAGAGCTTGCCTGCA
GAGAATACTCTGCCCACTGAAACTAAGGAGAGTGCTACCCTCCAGGTCTGCTCATAGAGGCTAACAGAGTCACCTGAAG
AACAAGCTCTTAACAGTGACAACTAAAACAGCTAGCTTCAGAGATTACCAGATGGCGAAAGGCAAACGTAAGAATCCTA
CTAACAGAAATCAAGACCACTCACCATCATCAGAACGCAGCACTCCCACCCCACCTAGTCCTGGGCACCCCAACACAAC
CGAAAATCTAGACCCAGATTTAAAAACATTTCTCATGATGATGATAGAGGACATCAAGAAGGACTTTCATAAGTCACTT
AAAGATTTACAGGAGAGCACTGCTAAAGAGTTACAAGCTCTTAAAGAAAAGCAGGAAAACACAGCCAAACAGGTGATGG
AAATGAACAAAACCATACTAGAACTAAAAGGGGAAGTAGACACAATAAAGAAAACCCAAAGCGAGGCAACGCTGGAGAT
AGAAACCCTAGGAAAGAGATCTGGAACCATAGATGCGAGCATCAGCAACAGAATACAAGAAATGGAAGAGAGAATCTCA
GGTGCAGAAGATTCCATAGAGAACATCGACACAACAGTCAAAGAAAATACAAAATGCAAAAGGATCCTAACTCAAAACA
TCCAGGTAATCCAGGACACAATGAGAAGACCAAACCTACGGATAATAGGAATTGATGAGAATGAAGATTTTCAACTTAA
AGGGCCAGCTAATATCTTCAACAAAATAATAGAAGAAAACTTCCCAAACATAAAAAAAGAGATGCCCATGATCATACAA
GAAGCATACAGAACTCCAAATAGACTGGACCAGAAAAGAAATTCCTCCCGACACATAATAATCAGAACAACAAATGCAC
TAAATAAAGATAGAATATTAAAAGCAGTAAGGGAGAAAGGTCAAGTAACATATAAAGGAAGGCCTATCAGAATTACACC
AGACTTTTCACCAGAGACTATGAAAGCCAGAAGAGCCTGGACAGATGTTATACAGACACTAAGAGAACACAAATGCCAG
GCCAGGCTACTATACCCGGCCAAACTCTCAATTACCATAGATGGAGAAACCAAAGTATTCTACGACAAAACCAAGTTCA
CACAATATCTTTCCACGAATCCAGCCCTTCAAAGGATAATAACAGAAAAGAAGCAATACAAGGACGGAAATCACGCCCT
AGAACAACCAAGAAAGTAATCATTCAACAAACCAAAAAGAAGACAGCCACAAGAACAGAATGCCAACTCTAACAACAAA
AATAAAAGGGAGCAACAATTACTTTTCCTTAATATCTCTTAATATCAATGGACTCAATTCCCCAATAAAAAGACATAGA
CTAACAGACTGGCTACACAAACAGGACCCAACATTCTGCTGCTTACAGGAAACCCATCTCAGGGAAAAAGACAGACACT
ACCTCAGAGTGAAAGGCTGGAAAACAATTTTCCAAGCAAATGGACTGAAGAAACAAGCTGGAGTAGCCATTTTAATATC
GGATAAAATCGACTTCCAACCCAAAGTTATCAAAAAAAACAAGGAGGGACACTTCATACTCATCAAAGGTAAAATCCTC
CAAGAGGAACTCTCAATTCTGAATATCTACGCACCAAATGCCAGGGCAGCCACATTCATTAGAGACACTTTAGTAAAGC
TCAAAGCATACATTGCACCTCACACAATAATAGTGGGAGACTTCAACACACCACTTTCTTCAAAGGACAGATCATGGAA
ACAGAAACTAAACAGGGACACAGTGAAACTAACAGAAGTTATGAAACAAATGGACCTGACAGATATCTACAGAACATTT
TATCCTAAAACAAAAGGATATACCTTCTTCTCAGCACCTCACGGGACCTTCTCCAAAATTGACCATATAATTGGTCACA
AAACAGGCCTCAATAGATACAAAAATATTGAAATTGTCCCATGTATCCTATCAGACCACCATGGCCTAAGACTGATCTT
CAATAACAACATAAATAATGGAAAGCCAACATTCACGTGGAAACTGAATAACACTCTTCTCAATGATACCTTGGTCAAG
GAAGGAATAAAGAAAGAAATTAAAGACTTTTTAGAGGTTAATGAAAATGAAGCCACAACGTACCCAAACCTATGGGACA
CAATGAAAGCATTTCTAAGAGGGAAACTCATAGCGCTGAGTGCCTCCAAGAAGAAACGGGAGACAGCACATACTAGCAG
CTTGACAACACATCTAAAAGCCCTAGAAAAAAAGGAAGCAAATTCACCCAAGAGGAGTAGACGGCAGGAAATAATCAAA
CTCAGGGGTGAAATCAACCAAGTGGAAACAAGAAGAACTATTCAAAGAATTAACCAAACGAGGAGTTGGTTCTTTGAGA
AAATCAACAAGATAGATAAACCCTTAGCTAGACTCACTAAAGGGCACAGGGACAAAATCCTAATTAACAAAATCAGAAA
TGAAAAGGGAGACATAACAACAGATCCTGAAGAAATCCAAAACACCATCAGATCCTTCTACAAAAGGCTATACTCAACA
AAACTGGAAAACCTGGACGAAATGGACAAATTTCTGGACAGATACCAGGTACCAAAGTTGAATCAGGATCAAGTTGACC
ATCTAAACAGTCCCATATCACCTAAAGAAATAGAAGCAGTTATTAATAGTCTCCCAACCAAAAAAAGCCCAGGACCAGA
TGGGTTTAGTGCAGAGTTCTATCAGACCTTCAAAGAAGATCTAATTCCAATTCTGCACAAACTATTTCACAAAATAGAA
GTAGAAGGTACTCTACCCAACTCATTTTATGAAGCCACTATTACTCTGATACCTAAACCACAGAAAGATCCAACAAAGA
TAGAGAACTTCAGACCAATTTCTCTTATGAATATCGATGCAAAAATCCTCAATAAAATTCTCGCTAACCGAATCCAAGA
ACACATTAAAGCAATCATCCATCCTGACCAAGTAGGTTTTATTCCAGGGATGCAGGGATGGTTTAATATACGAAAATCC
ATCAATGTAATCCATTATATAAACAAACTCAAAGACAAAAACCACATGATCATCTCGTTAGATGCAGAAAAAGCATTTG
ACAAGATCCAACACCCATTCATGATAAAAGTTTTGGAAAGATCAGGAATTCAAGGCCCATACCTAAACATGATAAAAGC
AATCTACAGCAAACCAGTAGCCAACATCAAAGTAAATGGAGAGAAGCTGGAAGCAATCCCACTAAAATCAGGGACTAGA
CAAGGCTGCCCACTTTCTCCCTACCTTTTCAACATAGTACTTGAAGTATTAGCCAGAGCAATTCGACAACAAAAGGAGA
TCAAGGGGATACAAATTGGAAAAGAGGAAGTCAAAATATCACTTTTTGCAGATGATATGATAGTATATATAAGTGACCC
TAAAAATTCTACCAGAGAACTCCTAAACCTGATAAACAGCTTCGGTGAAGTAGCTGGATATAAAATAAACTCAAACAAG
TCAATGGCCTTTCTCTATACAAAGAATAAACAGGCTGAGAAAGAAATTAGGGAAACAACACCCTTCTCAATAGTCACAA
ATAATATAAAATATCTTGGCGTGACTCTAACTAAGGAGGTGAAAGATCTGTATGATAAAAACTTCAAATCTCTGAAGAA
AGAAATTAAAGAAGATCTCAGAAGATGGAAAGATCTCCCATGCTCATGGATTGGCAGGATCAACATTGTAAAAATGGCT
ATCTTGCCAAAAGCAATCTACAGATTCAATGCAATCCCCATCAAAATTCCAACTCAATTCTTCAACGAATTGGAAGGAG
CAATTTGCAAATTTGTCTGGAATAACAAAAAACCTAGGATAGCAAAAAGTCTTCTCAAGGATAAAAGAACTTCTGGCGG
AATCACCATGCCAGACCTAAAGCTTTACTACAGAGCAATTGTAATAAAAACTGCATGGTACTGGTATAGAGACAGACAA
GTAGACCAATGGAATAGAATTGAAGATCCAGAAATGAACCCACACACCTATGGTCACTTGATCTTCGACAAGGGAGCTA
AAACCATCCAGTGGAAGAAAGACAGCATTTTCAACAATTGATGCTGGCACAAATGGTTGTTATCGTGTAGAAGAATGCG
AATCGATCCATACTTATCTCCTTGTACTAAGGTCAAATCTAAGTGGATCAAGGAACTTCACATAAAACCAGAGACACTG
AAACTTATAGAGGAGAAAGTGGGGAAAAGCCTTGAAGATATGGGCACAGGGGAAAAATTCCTGAACAGAACAGCAATGG
CTTGTGCTGTAAGATCGAGAATCGACAAATGGGACCTAATGAAACTCCAAAGTTTCTGCAAGGCAAAAGACACCGTCAA
TAAGACAAAAAGACCACCAACAGATTGGGAAAGGATCTTTACCTATCCTAAATCAGATAGGGGACTAATATCCAACATA
TATAAAGAACTCAAGAAGGTGGACTTCAGAAAATCAAATAACCCCATTAAAAAATGGGGCTCAGAACTGAACAAAGAAT
TCTCACCTGAGGAATACCGAATGGCAGAGAAGCACTTGAAAAAATGTTCAACATCCTTAATCATCAGGGAAATGCAAAT
CAAAACAACCCTGAGATTCCACCTTACACCAGTCAGAATGGCTAAGATCAAAAATTCAGGTGACAGCAGATGCTGGCGA
GGATGTGGAGAAAGAGGAACACTCCTCCATTGTTGGTGGGAGTGCAGGCTTGTACAACCACTCTGGAAATCAGTCTGGC
GGTTCCTCAGAAAACTGGACATAGTACTACCGGAGGATCCAGCAATACCTCTCCTGGGCATATATCCAGAAGATGCCCC
AACAGGTAAGAAGGACACATGCTCCACTATGTTCATAGCAGCCTTATTTATAATATCCAGAAGCTGGAAAGAACCTAGA
TGCCCCTCAACAGAGGAATGGATACAGAAAATGTGGTACATCTACACAATGGAGTACTACTCAGCTATTAAAAAGAATG
AATTTATGAAATTCCTAGCCAAATGGATGGACCTGGAGGGCATCATCCTGAGTGAGGTAACACATTCACAAAGAAACTC
ACACAATATGTATTCACTGATAAGTGGATATTAGCCCCAAACCTAGGATACCCAAGATATAAGATATAATTTGCTAAAC
ACATGAAACTCAAGGAGAATGAAGACTGAAGTGTGGACACTATGCCCCTCCTTAGATTTGGGAACAAAACACCCATGGA
AGGAGTTACAGAGACGGAGTTTGGAGCTGAGATGAAAGGATGGACCATGTAGAGACTGCCATAGCCAGGGATCCACCCC
ATAATCAGCATCCAAACGCTGACACCATTGCATACACTAGCAAGATTTTATTGAAAGGACGCAGATGTAGCTGTCTCTT
GTGAGACTATGCCGGGGCCCAGCAAACACAGAAGTGGATGCTCACAGTCAGCTAATGGATGGATCATAGGGCTCCCAAT
GGAGGAGCTAGAGAAAGTAGCCAAGGAGCTAAAGGGATCTGCAACCCTATAGGTGGAACAACATTATGAGCTAACCAGT
ACCCCGGAGCTCTTGACTCTAGCTGCATATATATCAAAAGATGGCCTAGTCGGCCATCACTGGAAAGAGAGGCCCATAG
GACTTGCAAACTTTATATGCCCCAGTACAGGGGAATTCCAGGGCCAAAAAGGGGGAGTGGGTGGGCAGGGGAGTGGGGG
TGGGTGGATATGGGGGACTTTTGGTATAGCATTGGAAATGTAAATGAGTTAAATACCTAATAAAAAATGGAAAAAAAAA
AAGAATATAGGACAAATAAGTGGTACTGTGTGAGTACAGTAATTACACCAAATAAATGGAAGGTGGTAAAACGTCTCCT
TGTCCTTTCAACAACCATGCTCCCCATCTGCAGGCATGCAACGCATACAAATACAAGAACACATACACACACAGCATGC
ATATATATATATACATATATATATATATATATATATATATATATACCATATATATCATATATATACAAACATACACACA
TACAAACATGCATGCACACATAGACACAAACATGTATACATACATGCATGCATGTACACACACACACCATTTCCACAGG
CAGGTATATCAATGGTGGTATTTCACAGTAGCCCATTAGATGTGGGTAAATCACTCTGGTTGGCTAACTCTGTTTAGTT
GAGGTCTGAATAAGCTTTAAAGACAAGACTCTTAGTTCTGCTGAATGGCATTTGCTGAGAGCTCACTTGTGGCTGCCAG
GAAGCTCTGTGGGTGTTGCTCATGTTGAATACGTGCCAGCCCTGGGTGTGGCAGATCTACAGAAGCACACTGCAGTGTG
GTTGCTGTGCACTGGGCTTCTAGAGACAGGCTTGAATTCAGCTGCATAAACTTGGGCAAGTTGCTTAGACTTCTGCCTC
CGTGTCCTCACCTGTGAAATGGAGCCAGTGATAACGCTCACTTTCTAAATGCTGCTGTGAAGCTTAAGTGAGGTTAATC
TTTGAATGCCTGGCATGTAGCAAGCGACCTGGGGCTGCTAACCACTGTGGCTCACCCTGTTCATTCATGAGCAAGAACT
GATCCGCTGGTATGTGCCAAACTCTATGTCAGTCATACACACAGACTTGCCCTCCCTCCCAAGCATGCAGTAGAGGTGC
ACACCCAGCTTGCATAGTGCTTTAAGAAGAGTCACAAAAACCGTCCCAAAAGTCATGGAAAGCAAGTGACTAGCACATG
ACCTGACCTGGATGGAGACCAGACATAGAATTGGGTGTTTAGAAAAGCCCCCAAGACCTGGTACAGTCTTAAAGTAAGA
AGAGATGTGTTTGGGGGAGGAGATAGGAAAGATATGTGTGCCTACAGAGGCTCCATGGCAACCAACAAGCCACCCTGGC
TTAGAGGAACATGCGTGAGCAAGGGAAATGTGTCTGAGAAGCAGTTGGGAAAGAGGGCCTGGTGGTCCAGTGGTTTCTG
CTAAGGACTTCAGCTGGATCCCTTATTGGTGCAATCAGAGAGTGGCACACTCAAATCTGTGTTTCAAGCCCAAGAATGG
TTACAGGCGTGAAAGGGAGGGGGTAGGGAGGATACTGGAGATGCAGTTCTCGGCCATTGATGAGACTGGCACAGGTGGT
GATTGTGAAAAGAGATGCAGAGAGGGGATGCTGAAGAGGATTCATGGCATGGCTATATGGCAAGATAACAATTGAGGAG
AGAGAGGTGTTGCAAACATGAGTGACTGTGTCCTCGGGTGCTTTTAAAAGAAAGGGAGGGCAAAGAGATCAACCTAAAG
CCCCAGCCTTAGAAATCAGGCTACAGGCCCAGGGTGAGGAAGTAGGAGAGATCAGGAGCAGAGCCTGGGGCCAGAAGAC
AAGGGTCCAGGGGTAACTAGAAAAAACAAGGCCCACTGGGAAGCCGAGGGACACTCAGAGAACTCAGCCCCATGGAGTT
AGGATGTGACCACGTGGAGTCAAGGACTGACCCTCTCCTAGCTCAGAGGACCAGGAGCATGGAGCCCACGAACGCAATG
TAGATTTAGCCACCAGAGGGCGATAGAGGGCCCGGGGAGTGTGGGGCAAGGACAGTAGGGGAGGGTTAGAAAGTAGAGT
GATGAAATAGCGGGCAAAGGCAGGCATCCTTGGCCACTCTTTGGGAAACTGGAATAACAAGGGGAGCCAGTAGATAGGG
ATGGAAATGCAAGAGGAAATGGTGCTGCCCACGGGGGTAACAGAATGTCTGTTCCTCTCCCCCCTGATCTAAGCCGCTA
AGGTGTTGGATTGCTTAACTGAACCATTTTGGTTGTTGTGGTGATGGTTGTTTTTCTGCCTGTCTTTCCACAGAACCGG
GTATAGAGAGGTCCAGGAAGTGGCGGAAGAGACTCCATCTCGTTCCACAGGTCCTGTTCTGATGCCCCGCTGGGGACCT
CATTCTCAGAAAGCCTCAGCAGTGTTCGGTATATTTTTAAAGCCTTGTGTATTTATCAGCTTAATTACTGATTTTTATT
TCACAGTTAAAAATAATAAAAATGTGTTTCCCAAGGTCATGTATCCACGACTACTAATTCACTAATTTAAAATAATTTC
ATCATCTCTCCAAAAAAAAACCTGTGCATATAAAGTCACACCCTCATTTCTCTAGCCCCAGGCACTAATCTAACACTAT
CTCTTTGACTTGGCTCATCTTAAACATTTTCTACCAGTGGGATCGTGCGGTGTATTTTGTGGCTGGATTCTTTCCACTC
AAGACAGTGTTTGCAATGTTCATGCATCTTGCCGTACCGGGGCCCTGTTCCATGTTCTACCAGAGTAAGAATCCATTGT
ATGGTGATGCTCAGTTGTGTTTACTTCTAAATTTGCAGGCATCCCCCCAATGGCTCTGCCCTTTTCCATTTCCACCTCC
ATGTGTAGGCTTCCAGTTTCTTCACATCCTTGAGAGAGTTTGTTATTGCGTGCTTTAATTACACACAATTGCAGGTTGG
GCTTGCATTCCTCTAATGACCAATATTGCTGGGTGACTTTTAATGTACTATTTATTGGGCATTGATACCACTTTTGGAG
ACGTGAGTATTCAAATCCTTTGTCTATGGTTTATTTGGGCTGACCTTTAATTCTTGAGTTGTGTGAGTTCTTCATATAG
TCTACCCAGACTTTTTTTTTTGTGTGTGAAATTCTAAGTGTTTGATTTCCCTTGGTGAGTTGTCTTTTTCACTCTCTTG
ATGGTAACTTCAGCCGCACAGGTCTTTTTACCTTGAAAAAGCCCAGCTTCCACACTCTTTGTTGGTTATCTGGGAAAAA
ATAGCTCAGTCCAGGACCTGAAGATTATATCTGTGTTTTCTTCTAGAGATTTTGTGGTTTGGGTTCTCACATTGACGTC
TGTTATCCATTTTGAGTGAATGCTTCGTTTACACTGTGATACAAGAATTCAAATTTATTTTTTCCATGTGGGCCCAGTA
TCATTTTTAGAAAATAACCTCTTTGCCTACCTTATTGTCTTGATGCTTGTCAAAGGCTAAGTGACTACAGATGTATTAG
TTCCAGACACTCAGTTCCTCTCCACTGATCTCTGTGTCTATCTTTATCCCTGACCACACAGTATTGATAACCGCATGAA
TGCAGTGAGCTTTTAATTTGGAAAGTATGAGTTCTCCAACATGCTTGTTTTTTCAAACTTATTTAGGCTTCCTGGAATT
TTATATTACCATGCTAATTTTAGGCTTAGGTTATCAATTTCCACCCCCCCCAAAAAAAAACTACTGGAAATTTGATATG
AATTGTGTTAAATATGTAAAATGATTTGAGTATCTTAATATTAAATCCTCTATCCCACGGATCCAGGATGTCTTTCCAT
TTTAAGGCCTCTAATTTCTGCAATGTTTGTGGGTTTCATTGTTAAATTTACTCTTAATCATTTTGTTCATCTTGGTGCT
GTTATGGACAGGATCGTTTTCTGTATTTCATTCTTGGAACATTCACTGAAGGGCTACAGAAGTACAATTGGCATTTATG
TTTTGATCATTATTCTGTTGTCTTACTAACTCATCTATCAGATTAATATACTTTTCCGTGGCTTCCTTAGGACTTGACA
TACCTGAGATCCTGTCACCTTCGAATAGTTCTACTGGGGTTTTGTTTGTTTGTTTTTTCTGATCTGGATGCTTTTTATT
TCCTTTTCTTGGCCAATTTTCATGAAGACGACTTCCAGTACAGTGGTGATAAGTGAGGACAAATAGTCTTTTCTTGTTC
TAGACCAGTGGTTCTTAACCTATTGGCCCCAACCCCTTTGGGAGCAGAGGGACACTACTAGACCCATATAAGCCAAGGA
ACAGCAAAGTAGAGCTGCATTGTTCAAACACCCCTCGCTCCTAAGAGAATTCAGTGGGAAGGAAGCTGCTCCATTGTTC
AGGTAATGATCAGTGGGATACTTGGGCCAGAACACAGTATGAACTACAGCCACAGAGAGCATGAGCGGGTGAGAAGCCA
ACACAGGAAAGGATGCAGCAAGAGAAACACTGAGAGCCAGTACAGGTGGAGGAGGCCAGGGAAGGGTGATGTCATGACT
AACATCGAGGTCAATATGATAAGATTTAGAATCATACAGAAAACACACCTCTAGTCACGTCTGTGGGGGCATTTACAGA
GAGGTTTAACTGAGGAGAGATGATCTACGCTGAATATAGGCAGTGCCCTCCCACAGGCAGGGGTCTGTGACTGAACAAA
AAGGGTAAAGCCAGCTGAGCAGCAGTATTCGTCCCTCTGCTTCCTGACTGTCCTGTCGCCATGCCTCCCCCACCATGAT
GGACTGTACCCTCAAACTGTGAGCCACAGTCAACCTTTCCTTCCTGAATTACTTTCATCAGGTATTTGGTCACAGAGAC
ATGTAAAGTAACTAATCCAAGAAGGAAGAAAATTATCTTAGGCAGTGACTGGGATCTCCCAAAATAAGAATGATTCGAT
GGAGAGGGGTCTGGCAGTGTGGAAAGCCATATTTTACCAGATTTCTGATGGTCTGTCTTGCTGTCTTCATCAAAGGCAT
AATTCCCAAAGTTGGTACAGATCTAACTAGGAAGAAGGTGACATTATCACAACTTGGAATTTAGCAGCCACATCCCCCT
TGGCCTTAATTTCAAGTTGTATCTGTCATAGGATCTGAGCAACCAAAGGTTCTCTGTCACTCAGACGGAAAATGTTCAC
AGTCACAATTAGTCCGCCAGAGAAGGTTTGCCCTGCACCAGAGTTGTCCTAAGGTGTCCCTCAAAAGGGGAATGGGGCT
GGCACAGGTGGAACTCCTATCATACTTGATTTTAGAAGCAGGAAGAACTCAGAGGTGACACAGTAGACAGATAGTAGCA
ATTGAGTGGCAGTTAGGACACAACCTGACTCAAGTATGCAACAATTTAATCCCTCGTGTATACAGTAGCCGTAGGGCCT
TAAAGAGATAGGAAAATCATTTTCAAAAATGTCTTTTCTCTTTCCAATAAATTGCAATAAAGGTAGAAATTACAGCTGC
ACAAGGCTCTGAGAAGTGACAGTTGCATGCTCAGCCATGGACAAGACATTTATACCATCTCCTCCAAGCTCAGGAAATA
GAGCAGGGGTTAGGAAGTGGGGAGATTTTAAGAACTTGAAGGTAGGGGAAAGGACTGCAAGATGCCATGGTCTGGGCAG
TCCACAGCCTTCGTGATGAACTCTCAGCTGCTGCAGGTACCTGCTCTGGTCCTGCAAGGGAATGGGCCATTCACAACCA
GGCAAGGATGGAGGAGAGATGCTAAGGCCCTATCTCTCTCTTCTTGCTGAGCCATGTCCTATTGAGTGAGTCACAGAAA
GAGGAGGGAGTCTTTTCTTTCAGCTGTGTACCTCTGATGGTGACCCTATCAGCCTCCAATAGATAATCTAAATCTAACT
GTTACACAGAAGGCCTTGGTTAACTTAAAGAGATGGTGGCACAACAAAATTTAGTCACGAATCTGGGAAAGAGGCAGGT
AGGGAGAAGGGAAGGCTGGTAGAGGTGGGAGTGAAATGAATGAGTTGGTAGAGGAAGTAACAAGAATACAGTGTGTGAG
CGAGTGTGTACATGCCTGTGTGTGAGCATGTGCATGTGTGTGTGCGTGTGTGTGAGCATCTGTGAATGAGTGTGTACAT
GCCTATGCTTGTGTGTGTATGTGTGTGAGAGCATGTCTGTGTACACGCCTGTGTGTGTGCGCGTGTGTGTGTGTGTGTG
TGTGTGTGTACTTATCAAATAACTTGCTTAATATAAAACAAAATCACTTCAATGGGCATCTTACAGTGTTACCCACTTG
ACAAGCTATCTGTCCCTGGAGGCATGTCTGACCAACTGAAACAGGCCAGCACTCACCTGGGTGTGTCTGCAGTCACACT
CAACCTTTCTCAGAGACAATACCCCATTCTTCAGACTGCCACAGATGATAAAACAGCATAGGGGAACAGGGATCCCACA
TCCACAAGTACCAAGACAAAGGCTCTCAGCATGTGCAGGAGGCATCCACAGACTCACTGCACACAGGAGAGCCTTGCTC
GGTTTCCACCTGAACACTCTCATGGAAGTATGACTCTGAGTTCTGTCTTCCCTTTGCACTGCTGCTGGAAATATTAAAA
AATGAACCCACCAACTCTCCATGAGGCCTGACCACACTCCCATGCTCCTACTGGCCCACCAACTCTCTGGCCACGCCAG
GCCACAGGGCTACCGCCAGGCCATCTCTCCAGAGAGCCCCTCCCCAAGTTCCTCTCACTACCATGCAATGCCCCATGCA
CCCCACAGTCAGCCATCTCAACACCTAATTACTTGGTAGAATCAAGACTCTTAAAGCCGAACTAACCAATCAAATTTAT
ATATCAATAATATCACAATTTACAATATGCCGATACAATAATTTCAGAGCCAGTTGATAATGATAAAAGCTTTATCCCA
ATGATCTAAACTTGCGATGCCATAACTGCTTGTGGCTGGGAACGTCACACAGGTTGACGTCTGCAGCCGGCTTCCTTCC
TCTCCCCTCTCTGAGAGGCTCTCTCTCTCTCTCTCTCTCTCTCTCTCTCTCTCTCTCTCTCTAACTCTTAGCTCTGCCT
CCCCTTTTCTTGTCCAATCTCAGGCCTCCTCTGCAATTAATGTAATTGGACAGGGAAAATCCTCCGACATTACACACTT
ACTATTTTCACTATGAAATATTTCAAATACATCAAGAAGTGCATTTGATAATATGACAAACATCCATGTGCCCACTGGG
AGAGAGAACAAAGGGTAACTATATTTTTGGTTTTTGCTTCAGATCCTTTTTAGAAGAAAGTAAAATATTACAGATGTAT
TGATGATCATCTCTATGAACCTTTTTCTAATTCCTCCCCTCTCCTTCTCCCCAGAGGAAGAGCTACTCTGAATTATCAA
CATATTCTACTTATTTGATTAAGCTATTCTACAAATACCTGTAGATAGTGTGTAGTATTGTGTTACCTGTTTTAGAATA
TTTCCTAGGTATTATGACACATCCTTTTAACCCCATGTATGCACTTAGTTTTACATGTTTATAATTAAATGTTTATGTT
TTTATGTTCAGGTATATTACATACCATACCATAACATTTGACATATATATGTGTGTGTGCATGTGTGTGTGTTATGTAT
ATGCTTACATGTGGGAAGCCATTCCGGACTCCCTGGCCAGCCCGGACGGAAATCTGTGCCAGGCAGTAAACAAGCCCAT
ATTTGGTGGATGGCCCGCCCTTAATCCCTGATTGGCTGAGCAAGCCTGCCTGGTGAGGTGATGTGACCTGTGGTGATTG
GTTGAGGCATGGTTGTGGCTTGAGTGGAAAAAAAAAAAAAAAAACACTTGTACCCCGAGGCCGGGAGATTTGAAGAGAG
AAGCTGCCTGAGTAAACTGCTTTAAGAAGAACTGGTTGCGGTTTCCTTGCTGGTTGAGTTGGACGCAACAAGTGGTGCC
GAAACCCAGGAACCGACTCCCCCACCGAAGAGGTTCAGAACTTTCTGCAGTCAGGGTCTGTCGACAGGATGAGTACGGT
AGGTAAATAACTCACCATCCCGGGGTTGGGATTAGGCTCTTGGTACGAGACAATAAGGCTCTCCGGCTCTGGAGATGAG
AAAGTGAAAGTAAGAAAGACTAGGTAGGAAAATTTTGGTAAACAGTGATAGGAAAATTTTTGGTAAACAGTGGTAGGAA
AAGTTTTGATAAACAGCGGTAGGAAAATTTTAGTAAACAGCTATAAGGAAATTTTTGATAAGCAATGGTATTTAAAGAA
AGCACAGGCCTGTAAGGTTCCCGGGGGGGGGGAGGGGGGACAAAGCATTAAAGTTCTCAGTTAGAGACTATTAGGTTCC
CGGTAAGTGGGACGATTCAAGTTCTCGGTTAGAGACTATTAGATTCCCGGATTAGGGACTATTAAGGTTCTCAGATAGG
ATAGCACAACTTTTAATCATCCCTAGTCTTCATGGCTTATGCCCAGCCACATCACAAAAAAGGGGAGAAAAAGGATTTG
GTTCTTCAGGCACCCAACTTACCTGTCTGTCATTACAAATGGATCAGCGTCCAGTACTGGAGTTACAGGTGGATGGAAA
ATCCATCTTGGGGCTTTTAGATACAGGAGCTGATAGGTCCATAATTGCAAGGAAGGACTGGCCAAAAGGATGGCCGATT
CAGGAGTCAGAACAAATATTACAAAGCCTGGGCTATGCAAAGAGCCCAGACATAAGTGCCAGGATGCTCCCTTGGAAAG
ATAAGGAAGGGCATGATGGGATGTTTCAACCACTTGAGCTGCCCATTAACTTATGGGGTAGGGATTTGTTGAAAACAGT
GTCCTGTTTAGGAGCAAGGATCGCCCCTACGGCCATTTGGCCTCAAAAGATACAGTTGAGGGTTGATTCCTTGCATACT
TTAAACGATTTTCAGAAATTGTTAGGCGATATCAATTGGATTCGGCCCTATTTGAAGATCCCAAATGTTAAATTAAAAT
CTCTGTTTCAAATTTTAGAAGGAGACTCAGAGTTAAGTTCCAAAAGGGAATTAACACCTGAGGCTAGAAAGGCCCTTAC
CCTAGTGGAAGAAGAATTGATTAAAGCCCAGCTTCAGCGTTGTAGAGAAGGGTTACCCATCATTTTGATAATATTACCT
ACTGAGATGCAACCCACAGGATTATTGTGGCAAGAAGTCCCATTGTTATGGATCCATTCCAAAATATCTGCTGGGAGGA
CATTAGATCATTACCCTACAACAGTGGCTTCTTTAGCCTTGTTGGGAATTCAACATTGTTTACAATTTTTTGCTATAAA
ACCCAGCTCTATTGTAATCCCTTATGATGGGTATCAGCAAAAGGTCCTAACAGCAAATATTGATGAGTGGGCTATTTTA
GCCTGTATTAATGAGGGCCCATTTGATAATCATTATCCCAAACATCTGTTAATGACCTTTTTTAAGGAACATCCAGTCA
TTTTCCCTCAAGTGGTGAGGAAAACACCCATTAAGGATGTAGTTAATGTGTTTACTGATGGATCAAAAACCGGATGCGG
AGTATATGTGGTAGAGGGGAAAGATCCTGTGGTCAGGCAATATTATCCCTGTACTCCACAGATGGTGGAATTACAAATA
GTTATTGAGGTCATTAAGAATTGTGATTTTCCTTTTAATCTAATTTCAGATTCACAATATGTAGTTAATTTAGTTTTAT
CCCTAGAAGTGGCTGGACAAATAAAAGCTCATAGTACCATCTAAGCCTTGGCCTTACAATTACAGAAGTTGCTGTGGGA
AAGACACCAACCATTATATATTGAACATGTCTGTGCCCATAGTGGGCTTCCAGGACCCTTAAGTAGGGGAAATAATATT
GTGGATAATTGTACTCATATGGAGTTCATATTTTTAGCATCCCCTAGGGAGTTAGCCTCACAATTTCATTCTACCCAAG
TGGATCCTGGCTTGGCATCAGGAAAGAATGGGCTGGGATAGGTGCCCTCGCTGTGGCCCTTGTTTTGGCATGCATGTTG
TGTTGGTTGCGGAATGCCCAGGTCCGCACTGCCGCCATGGTGGTACAAGCTTTTGCCACAGTGGAAGCAGGGCAGTCAC
CTCAGATTTGGCTTGCTGCCATGAAGAATGAACAATGAAGCCATTGCTTAGTAGGGTTGTAAGGCTAAGCACTGCACAG
AGGTTAGTCTGGAAGCTGTTTGACAATGTCTGGGAGGTATGTCTGATTGCATGAGGGTTGAGTGTCCTAGTGTCCTTCC
CCCAATGGCACGAGAGCAGGTCAGGGTTGCTCTGGGTAAAAATCTGTGAACCTAAGAGTCAATCCTGTACATGGCCCCT
ATTTCTGCACACTGGGGATCAGACCTCTATCTTCACCCACAGAGCTTGCTTCAAAAGAATATAAAAGGGGAATTGTGGG
AAGCCATTCTGGACTCCCTGGCCAGCCCGGATGGAAATCTGTGCCAGGCAGTTGGTGGATGGCCTGCCTTTAATCCCTA
ATTGGCTGAGCAAGCCTGCCTGGTGAGGTGATGTGACCTGTGGTGATTGGTTGAGGTATGGTTGTGGCTTGAGTGGATA
AAAATGCTTGTACCCCGAGGCGCGGGGGATTTGAAGAGAGAAGCTGCCTGAGTAAACTGCTTTAAGAAGAACCGGTGGT
TGCGTTTTCCTTGCTGGTCGAGTTGGAGGCAACACTTACATTACATGTTTATGATTCAGTTTTCTATGTTTATGGCTTC
TGTAGATCTGAACCATCTGTGGTAATTCCTGTTAGTATTCTCATGTGAAAGGGCTCCATGGTCTATAGAATCATACCCT
GACATAGCTGTTGTCCTGGTTGGTTTTTTAACAACTTGTTACAGGTAGAGTTATCTGGAAAGAGGAACCTCATTAGCAA
AAAAAAAAAAGTCTCCATTAGATTACCTGTACGCAGTAAGTCCAGAAGGCATTTTCTTGAGTGTTGATTGATATGTGAG
GGCCAAGACCTCTGGAGGTGGTGTCACCCCTGGGCAGGTGGTCCCGGGGCATAAAAGAAAGCAGGCTGAGCAAACCAAT
ACACAGAACTTCTCTATGCTTCTGGTTCATTTCCTGCATCCAGGTTCCTACTTTGAGTCTCGGCTTCCTGCCATGACGG
ACTGAACAATGAGCCAAATAAACCCTTCCCTCCCAAGTTGGTTTTGCTCACTGTCTTAATACAGCAATGGAAAGAAAGC
TAAGACAGTTGTTTTCCTGTTTGTCATCTTATCAGCTCCAACCTGGTGAGTGACCTGAGCTGGTCATGGGGTACGCATA
GTTCAACTTTACTAAATAGTACCAAGTTACCTTCTAGATTGCTGGAAACATTCTTGGCTTAGTTACTTTTCTCATTGCT
TTGAGCATATACCTGAAAAACGCAACTTGTTGTTGTTGTTGTTGTTGTTGTTTTCGAGACAGGGTTTCTCTGTGTAGCC
CTGGCTGTCCTGGAACTCACTCAGTAGACCAGGTTGGCCTTGAACTCAGAAATCCGTCTGACTCTGCCTCCCAAGTGCT
GGGATTAAAGTTGTATGTCACCATGCCCAGCCTGACAAACGCAAGTTAAGGAAGGAAGGGTGGTCAGAGAGTATGGTTC
ATTTACTGCCTGGGTCTGCCAGCAGGAGCAGGAGGTGACTGCTCGCTTTGCTTCCAGAGTCAGGAAGCAGATAGAGTGA
ATGCTGGTGCTCAATTCACTTTCTTTACCCGGTTTGGGGCCAAAGCTATGGGGTGGGGTTCCTCCTCCATTACACCTCT
CTGGAGACACCCTCACAGGCACACCCAGGTTTCCTAGGTGATTGCAAACACTGAGGTTTCACCAACACAACCCTCTACT
CCCATCAGTCACATACAAGCAGTCTGTGCACCTGCAGCAAGCCGGACTTCACAGTCCTCAGGTGAGCTGTGGGAACTCA
CTTCCTTACAATAGCATGAGTTCCCGTTCCTGAGCCCAGTCTGTAAGGGGCATCCCTGCTCTCAGGAAGCCCCACCTTG
ATTTCCTCTGCTTCTCTGCACCCTGCTTTCTTCACACTGGGGGCTCAGCAGCTGTCATCTAAGGTTGCATTTTGGGAGA
AGCATGTACAGATTGAAAGCAAATAACCCCTAAAACAGTCTGTGCAAAGAAGTCAGAGGGCCAGGAAAATCTTACTTGA
TTGAGATTGTTGACTTAGTTCAGCTACTGTAAGTTACTGTAGACTGGGTAGTTTATAAACAATGGTGGTTGATTTCTTG
AAGTTCTGAAGGAAAGAAGTCCATAGTTTAACAGACTAGAAATTCAAGACCTATTCTAACTGAGCTCCTGTGAGAGCCT
CCTTCCAGACTGCAGGCTGCCAGCTTCTTGCTATGTTCCTATACTGCAGAAGCCGGGAGAGAGACCTCTGGGGTTTGTT
TTTATAAAAGAACTAAGAACATTCGTGACAAATTACCTCCTTAGGCATCCCCCACCCCATGCCTGTCACACACTAGGGC
TTTAACATGCGAGTGTTACAGGAACATGGCCATACTGTCTATAGCAATCATCTCATTAGTAGTAATTGAAAACCTTGTG
TTCAGAAATCATTTGTCAGAACCCAAGCCCAATCTTTTTTTAAACAAACATCGGTACTATTTTTAGCTTAGATTTTTCA
TTTGACTATCCAGGCAAGTCTATCCCCCCAGAAGCCATGGATCAATAACTAAGGAGTTTTACCATTACTGAAACTATCG
GCCTCATCAATAACTAACACAAACTGAGTCACAGTTCACCTGGCATTGCTTTGGCACCCGCCAACAGCCTAAGACAGCT
ACACCATAACAATGAAAGGCTAGCTGGCCTGGGAGCCTGCTGGTTATTACTGCACTGGCAGACATTGGGAGTGTGTGCC
CTCAAAGGTAGACTGTGAGGCCAGTCTCCCCATATGGTGTTTCACAGAACCTCAAAATCAGACCGATGAAGAAGGAAGG
TTTCTTCACCAAATCTGTGACTTGTGTTCCTCTACCTACACCACTCCTCGTCCCTTTATCTTCTTACTGCTTTGGTATG
TAGACATTTTTTGGGGGTGGGCATGTAGTATGTGAGAGGGGAAGATGTTAGAGATGGTGCAGAGGAACGGATGGTACAG
GTGATGAAGGATAGTGGTGGTGGTGGTGGTGGTGGTGGTGGTGGTGGTGGTGGTGGTGGTCGTGATGGTGGTGGTGGTA
GAGATAGTAGTGGAGAGGATTGTCCTGAATAATGTTAGGTCAACTTGACACGCACTAGAGTTATCTGAGAGGAGGGCAC
CTTAATGAGGTGCCTCCATAAGATCCAGCTATAAGGCATTTTCTTAATTAGTAACTGATGAGGGAAGGGCCCAGCCCAC
TGTGGGTGGTGCTATCCCTAGGCTGGTAGTCCTACATGCTATAAGCGGGCTAAGCAAGCCACGAGGAGCAAGCCAATAA
GCTGTACTCTCCATGGCCTCTACAGCAGGCCCTGCTTGAGTTCCGGTCCTGTCTTCCCTTGATGATGAACTGGGATGTA
GAAGCATGGGTTGACTCAATTCTTTCCTCCCCGAGTTGCTTTGGTCATGATATTTCATCATAGCAATAGAAACCCAAAC
TAAAACAATGCTCGTTGTGGGACCGTGAAAGGCAGACTGTTTTCTAGTTGAGCTAGGTTTAAACCCTGGAGTCCCAGCA
GATAACTCTGCAAGGGACAGAAGGAAGTTTGCCATGCTCGAGGCGCTAGCTCCGTAATTCTGTGGCCATCAGTCACATA
GGGCAACATCCCAAGCTCCTGGTATTCTGTCTGGACTCCACCCCCACAGTTACCTGGCAACAACCAGGTATGCTCCTCC
CCACAGTTACCCAGCAACAACCAGGTAGGCCTGGCCCACTATAAAAGGGGCTGTTTGCGCCCTCCTCGCTCTCTTATTC
TCTTGCTCTTGCTCCCTTGCTCCCTATCTGCCTTCCCTTCCCCTCTCTCTCCACGTGGCCATGGCCGGCCTCTACTGCT
CTACTCTCTCCCTTTCTCTGCCTTTCTACAATAAACGCCTTAAAACTATGAACTGCCTCTTCTCATCCAGATGCACCAT
GCTAGAGCAATGGAGCAGGTCTTCCTCTAAAGAGCTTTGTCTAATCTCTCGGAAGGTCTCCCTGCCTCCCAGACATGAA
CGCCACCAAACCAAGCGCAGACCCACACTCCATTCTCAGCACTCAGCAACATCTGCAGTGCCGTCCTCGGCCTCCATGC
AGAGACCTCAGACTGTGCTTCCCCACCCCTGGAGCTGGGTCCTGAGTCTTCTCACAGCCTGACACCTTTGCAGTCAAAC
TTCCCGTGTCCACCTCCACAAGCAGCCCTAGCCCTGTGGCAGGCCAGATGCAGGACACCATTTTAACCCACAGCTCATA
AGTTTTTGTGTGGAAACTACCACATATTAAAGAGCTGGATGTTAGTGCTGTGCCTCCTGTAATGCCTCTACTCAGGACC
TCAGGCTCACTGAAATGCAGTTTCTCCTCTCTCTCTCTCTCTCTCTCTCTCTCTCTCTCCCCTCCCTCCATTTCTCTCA
AAAAGTATTAGCATAGGAATTTGCCTCAGGCCCTAACAGGAAGGATGCCTACAATGTCTAACTGCCAGCAGCTCACAGA
ACACCTTTAGCTGGGATTTAATGGGAAGGTGACTGCCTGGGCTAGAAGCTGGATTTGGACTCTTAGCCTGGGATGTAAC
AGAGCCACTTAGAGAGGCAGAGATGGAGAAGAGAATAGAGAACGTAGGTTTTAGGGTCAGACAGACCTGAATTCTGCTG
TTTATTTTCTCTGAGACCTTGGCCATGTTACCTGTCATCTCTGGCCCTCACTGCTTCACCTCTGAGGTTGGCTCAATAA
TGATCCATGGTCTACTTAAGAGAATGAAACAAAGCAAGGCTCAGCAGAGTCCAGCAAACATGTGCTGTCGCTGACCTAC
CCCACTGCACATAGTACTGGGACTGAGTCAAGACCTCTAAGGTGATGCTGCCCACCTGCCTGTGCAAGGGCAGAGAGAG
CATCCCGCATCCCATCCCATTCTGGTGGTTGCAGACGAATGAAACAAATGATGGAGAAGCTGCCCACTCATTGAAGAAC
TCAATTCAGATCCCTCCTTGCTCTGGCTCCAGCCCAACCCTCACCTACTCAGGGACTTCACAGGTTGGGATGAGAAGGC
TTGAAGGAAGGAAGGAAGGAGGGAGGGAGGGAGGGAGGACCAATCCTGGTGAGGGAGTGGGGTGTATCTACCGGTAGTT
CTATCCCTCCCTTCCCACAGAGCCTTCTAGGAGCACGCTGTCCATGCAGGCTCCTGGCCTCAGCCCGACTAGAGTCAGA
TTCTAGAGCACAGCTTAGATGCTAGAACTGATTCTGCTGCCCATCAACACCTGACAACCACTGGCTGATTGCTGGTGCC
TGCGGGTAGAGGCTGCCACGTGACATAGTCGCTTGGCGTGCTGAGGCAGAAGGGATGAGTTCGAGAGCATCCTTTCCAC
CAGGGGGCGTGCTGAGAGCAGGGCTACCCCAGCCCACCCCCACCACAGAATGCAGCCCACTCTTCACCTGTCTCAGAGG
ATTCTTGGCCTCTCTGCCCAAGTGACCTCTGCCTCTCATCTTAAGGATCTCTCTCCTATCTCCATCACTCTCTGTCTGC
ATGTCTCATTCTCTGATTCTGTCCCTTTATCTTTCTCTCCCTACTCCTATTTCAGGACCCCTGTCAGCTCTTTCTCCTT
CTCACTCACTACCGCATCCCTCTCCCCTCCCTCTCCCTCTATCTTCTCTCCCTCTCTTCTTCCTCTCCCTCTCCCTCTC
CCCTCTCTCTCCCTCTCTCCTTCATCCTCCCTCTCTCCTCTCTCCCTTCTCCTTTCCTCTTCTTCCTCCCCACTTCTCT
CCCTCTCTGCTCTTCCTGAGCCTATCCATGGATCTGTCTTCCTGCTCTGTGTGTGTCTTTCTCCCTGTGTCTCTGAGTT
TCCCTGTCCCTTCCCTGTCTCCTCCCTCCCTGGAGGCTTCCAGGCTGGTGACTGGGAACAGATGGCCATTAGGCTTTGG
CCAGAGGCCCCTGTCATCACTGAGGCTCCTCCCTCGGTGGCAGCTTGTCAGAGACCACCTGGGGAGTGCAAACGGTCCT
GCCACAAACAAGTCACTGTCACCAGTGATTCAGGGCCTGGCCCTGTCTAATCTCGGCCTGCCTTGGGTAACCCTGTGAG
TGGCCAGCTCCCTGCCTTGCTTGCGCAATGGCTGCTGTAGGGGCCAGAAGCCAGGAGTGGGCTAAAGAAGGACAGTGGG
AAGTTTTGTACCCAGTAGGCCTTGCCAGGCCCAGAGGAAGCACCTGGAGGCAACAATCATCTGTGACCCATGGCCACAG
TAGTTGCGATGCCCTGGGCCTGTGTCCTATCACCAGCCTCCCATATCGGAACATAAGAGATGAAACCAGGCTGCTCAAA
TACCCCCAAGCCCCTGCCTGATTTCTCCTGGACCTGCATGGCGGAGCTAGAGTGGCCTTGTTTAGGTGCCCTGAGGGCA
AGCCCTAAAGAACACTGCAGTATCCAGTGAGAAGCTGGTACCCTCACCCCGCCATGGCTCTAGCTGTGCCCCCACCACC
TGAAGGGTCCTGAAGTTCAGGTAGCCTAGCTGCACCCATGTAGGTACACATATACACATATCACACACACATACATCAC
ACACACACACACACACACACACACACATACATCACACACACACGCACATGTGAATTGCCATAAAGCAATACCTATCATA
CACCAGAACACTTGTTCTCTAAAACATAAACAGACAGGCAGGATCATCTCTAGAAAGGGCCCTGTTACAAATCCCTGAC
ACACACTCCTGCACTCTAGTACACCTGAGATACCAAAGGGAGATGCTGCTGTGTGGCCTGCTTGCACCCCTGTTCTTCC
CATATCCACACAGTAGATGCTCTGAGGGGAAGGTGAGCATCTGTGGCCGCACGCCTGCCATCCCCCTGCAGTCCTGCCC
CAGGTTCAAGGACAGTCTGATGTCACCCATGCCCTATTCAATACAAGGCATATGCTCCTCTTCCTATCTCTCATCTATG
TGGGCAGTGAGTAAGATGCTCCTGGTTTACCCAAGAAGGCTGAGGCTGCAACTAGCCTGGAAATGCAGATACTTCCACC
TTGTCCTTCCTAGATGGTCTTGTAGGAATCCAAAGAAATCTTGTAGGATACAAAGCAAAAGCCCCTATCAGGAGCCCCC
CTGATACAGAGGTGCAAAGGGGATAATTGAGAACCACTGGGCTGGCCCCACCTCGCAAGTGTGAGCTGCAGGAGGCCAA
GTGGACCGATGTGCAGCCTCCAGGAGGAAGGCTTGGGTCCTGCAGCTGCCATGCACCCACACCCTACGAGCTGCCTGCC
CTGCAGCAGCCAGAGGCAGTTGGGCCTGGGAGCTCTTCCCCAGGCCTGTCCTCCCCTGTCCCCACCCTAGTCACCCCCC
TCCTAGTTGGAGGAAGGACAGGATGGCAGGATACAGTGGGTGCAACCCCGGCTCATGTTTCCCTCCTCTCTTCCTGGCA
GGGATTACACGGTGAGCAAGGAGGACAGCTGAGCTGGCAGGTGGGTGGCCGGCCATGCTGAGGGGCGAGCTGGGGCGGA
AGGACGCGACAGCTGAGGTAACTCGGGCCTTTCATTCCCCTTTCCTACATCTCCCCTCCTGGCTCCCCTGGGGAGCTTG
TACATCAGGAATGAGTCTATCAGACCAAGAACCCACCCATGTGTAGGCACCTCTCTTGCTTTACCCTAATTCCGCTCAG
AATGGGCAAAAGTAAAACACGTAGGTTTAAAGAATTGAACTCAGAGAATTGAAGATTTGTGGTTTGGGGATGGGTAGTA
GCAGGCCTGGAGTGGGTGAGGTGTATCCAATTTAAAACCCACTCATTAGCATTGACACTCCTCCCTCTCCCAGCTTCCA
CCACAGCCCCAGTAGAACCGTCTCTACGGCTGTGAGAAGGCACCTGTCCGTACAAATGCAACTGCACCTTATCCCAGCC
TTTACCCTGATATGGTCCCGAAGCCTGTAGTTTCTTGGGCTACCTACCTGACCATTTGGAAATACTTAGCTTCAGGACA
GAGGATGTCAATCGTTGACCCTTAGATACTTATTTGAATGACTGGAGATAGTTTGGGGATCAAGCAATAAAAATTAGGT
ATGAACCTAGAAAATTCAGGAATCATGTGTCAGCAGAAAGCTATGACCTAGGCTCAGGTCTTGGTGGAGGCCTGAGTGA
CAGTCTCGTGCAAGGGAGGTAGGAGGAGGCCCTGCCCTTCTCCTTCCCACCCCACCCACAGGAGGACATCCACAGTCAG
AAAAGCAAGGGTAGATATTGAAGCTGTTGTGTGGATGCCCCCAGATCTGACCCAACATCAGCTGGTATGTTCCAGGCAG
CGACAGTCTAGCTGATCTCTGGGTCTGCTGCAACCACGAAGGGGTGTTCCAATGCCATGGCCAACATGGAACAATCAGG
TACTGGACTCCAACAGCTATGCGTGCTCTCTAACCTCCTTCCCATATGGTGCTGCTTGAGCCGGTAGACTCCTTGGTTC
CTCTCTATATCTACCCTGAGCAGTGCAGTCCAGAAGTTCCTGCCAGGTAGCATGTTTTCCATCTGCCCTAGATGTTTGT
CTCAAACCCTCATTCTGGTGTCCTATATTAGCAAGCCTGTGCAACCCAGTACTGTTCTTCACACATCTGTGCACATTCC
ACAACGACTTCTCTGAGCCCTGCTCCATGAGCGAAATGTGTTAGCTACTTGAAACGTCTGTTGGAAAACATTCAAGACC
AAGTAAAAGCACCTGGCACAGGGCGCCGCATAGAGCAAGCCCCAAAACATGTTTCTCCTTTTCTAGTCATAACGGCTAT
AACAAAATACCATTAACTGGGAAGCCTATATATTAGGGGGGAAAAATTCTCATAGTTCTAGAAACTGAGACGTCAAAGA
TCAGGATCTCAGAAAATTTACCATCTGGAAGGCCTACTTCCTGTGTTAGAGATGGCACTTTCTCCTTCTGTCCTTACAC
GCTCATCTGGGATAGTTTGCTAAGGATACTAATACCATTCATAACTGAATCATCTTCCAAAGACCCACCTGCCATCACT
ATCACACTGATGATTAGGATTTCAATATATGATTAGGAGTTGAAAGAACACAAATGCTCAGGCCATAACTGACACTCTT
CTCAAACCTTCTTGCCTGCCTGCCTTCCATCCTTCCTTCCTGCCTGCCTGCCTTCCTTTCTTCCCCATCTCAATCCTTC
CAGAGAGTCCTGAGCATCCTAATTAATCCCCCCTCATCCGCCTCTATTTCACTCCATCCAGATCCTCAGTGAGCCTCTG
AGGCCCCAATAAGCTCTGTTGCCTTTCTAAGTGGAGCTGCACACAATTTTCTAGGTCAACTACAGAGAGGGACTCATTC
ATCTTTGTGTTCCCAGGCATCCAACGCAGACCTAGAACATAATGTCTGGTTCCAGATGGAATGAAGGGGTTGTGATTGG
ATGGGGATGGAGGACAGATGGACAGATGATGAATGTGTGGGTGAATGGATTGGGTTAGATAGAATGGATACAAGGATAG
AACTGGCAGATGGGTTGAGGACCTGTGTCTACATAGAGATGTAGGGATGGGTACATTAACAGCTAAGTGAAATGGATAG
ATGCGAATATGTGGTACCCAAGTATTGAAGAGGAGGGATAGGAGGATGGAGAAACAGATGGCTGCAGTCACATGCCAAG
TTGGCTTTGGGCCAAGACAAGAAGGCTTCAAGGAACTCTCTCTAATACTTTCACCTCAATCCTTGTCACATCCTGTTTG
CCATTGCTGTCACAGCACAAACTTGGGGTGGTGGTGACTGACGGTGGTGGGTGGTGGGTGTGGATGCTGCCTCCATTTG
CAGTTTTGCCCAAGACCTCACTTATTGGTCCCTGCCCCAGGCTCCATGTACCACAAAGCTCAGAGCACAGGCATGGGTG
GAGAGCACACTTGCCCTTCCCCAGACACCTTCCCAGCCCTCAAAAGTTGCCCTGGAGACGGATAACTGAACGTACCAGT
GGAGATGCTGCTGTCCTCAGGCTCAGCACCTAAGTCTGGACACCAGCCAACCAGACTTCTCCCCAGGGTGAGCTTGGAT
TCCAGCCACATGGAGGTTCAGCTAGGCTGACACTCCTATGAGAGGCCTCAGTAAATCCAGAGGTCCTTGAGAGCAGGCA
TGGTATCACCCTCCCTGCCCAATCCAGTCATGCAGTCTTTGACACAGAGTTGGCAGAGCAAATGTTTGCGGGCTAAAGA
AAATGAGCAAGGCATCCTAGACACAGCCAGCTGTTGCTACGAGAAAGCAGTGGTACGTGCTCATCTGAGCTGAGAAGGT
GCACACCAGCTCTGCCGCTTCCCAGGTGGGTGACCTTAACTGGTCCCTGGACCTCTTTGACCTAGCCTCTCTTCATCTG
TAAACTCATAATAACGGGTGACACCACTCAGCCCTCGCCTGTGCTTGCTTGGACCACGCAGAAAACCTCTGATGTTGGT
CTTTTGAAGGACCCTAGTCTGAACCAACAACAGTTCCTGCCCCCCACTCTTCTCCACCAGGGCAGGGCAGGCCTGTATA
GAGGAGCTGGTGAGTTGCAGGCACTGAGCATACACCATTTCCAGGTTTCTGCAGCCAGGGCTATCTATAATCACCCTGA
CCCCAATCTTTCCATGTGAATTATGTAAAGAGAGTTCTCCAAAAAAAACAAGACCCATGAAAAGTCCCAGCCTGTGCTT
TCCAGCCCTCATGTCATCTTTCCCAACTTGATAAGGACCATGGCTTATTAGTCATTCCTACCCAGCTCACCCTGTCCTG
CGCCTGCACACGTGAGACTAAAAATACCCATGGCTCCTATTAGACACTCTGCCCTAGGGACACGCCAAGCCTGAGTCTG
CAGTGACACCAAAAGCTGGCTCCTTCACAGCCCTCGGATCCAGGCCACTGGGCTCTTTTCCCCAGCAGCACCCTCCTGA
TGGGCCCCTGAGGGTCCATGGGATTGATCAAAAGGCTGCTGTGGGTTTGGTTGTAGCTCTGTGTCACTCTCCAAAGCCT
CCTCCTCCGTCTGACTCATTTGCCTTGTAGACAGCTTCACCCCGCCCAGAAAATGGTTCTTTCTCAAAGAACTCTCATC
TCTCTGAGTGCTTCCCTGGGCCCTACAGATATTCAATCCAAACCTCAGCCCCTGGAAAAGACACCCGTGCTTCTCACTG
AAAACAGATGCGATAACAAGGGTGTTGGGTTCAACGGGAAAGTCAGACAGGCCTCCCCGAAAGCCTTGGGGTAGACTCA
AACTTGTGTGCATTGTGTGTGGGGTGTGTGTGTGTGTGTGTGTGTGTGTGTGTGTGTGTTGTTTCATGTTGGTGAACGT
GTTTACACGTGCATGTAGGTACACATGCATGTGTGTATGTATGACTGTAGAGGTCAGAGGTCAATGCCAGTTGACTTCC
TCAGCTCTCATGTTTGTACAGCAAAAATCTTTACTGACTGGGTCATTTCCCTAGCCTCCTGAACCAATCTCTTCATCCT
TCATTGTTTTCTATCAATAAAATGGACACAAAGCCATAGGTCCAACCTCTAGCTGTTGTGAGACAATCAGGATAAATCC
ATGGGAAGAAGAACACAGGCTCTGAGCTCTAACCCAGAACTGGCACTGAAGCGGGAACAAGGGGTGGGCAAGGTCTCTT
ACTCACAAGGAACATGCCAGGGACATGCCAGGGACATGTCAATGGGCCGGTGATGTATGCTTGGCAGGGCCGAAGAGGT
GATCTCTCCTCAGGCTCTTATGTGCTACTGGATATCTCTCTTGGCATTGACCAAGCTATTTTGGAAATGGTGGTGAGAG
ACGAATTGATTTTCATCATCTCTATACCCAGGAAAAGGCCAAGACCGTCACAGAGCGGGACTCCTATAATCAAAGTACC
TGAGGGCTGGACCAGAGTGCTTCCAACATTTACCTGTGAATGTATACGGCAAAACATGACTATACCAATCTCTGGAGAG
AAAAAACAAAAACAAAAAACAAACCAAAAAAAAAAAAAAAACCCAGAAAGCTAATAAAGGGGTCACAAAAAGCTAGGCT
GAAATGGAAAGCAGTGAATAGCAGGCTGGGGAGGCAAATGCCAGAAAGGCTGAGAATGGAGATTGTTAAAAAAAAAAAC
AAAAAAAAAACTCATGCTTTGGCTCTTGGCAGCAGCCAGGGTGGGGGTGTGTGAACAGGACTAAGCAACTCTCTGAATG
AAGCTCTCCCCAAATCTGTGCTCCAGAATGTTCTGCCAGCAGCCAAGGAAGGTGTCTTTAGAGACGCTTGTCCTCACAT
AACCTTCCTCTCCTCTGGGCACTGAAGGTCTGCGAGAACGATGCAAAGGAAGGAGCAACTGCTCTGCGGGGCTCAGTGC
TTTCTCCAGGCTAAAGCCAGAAGTGCCAGCTTGCTGCAAGCTCTCATTCTGCCCAATTTGGCTCAAACATTTATTGAGT
GCCTCCTGTGTACATGGGGAAGGCAGAGGGCTGAGGAGCAAGGGAGGCAATGACCTGGTCCTCAAAGAGGACCAGGTCT
GGAGAGGACGTTATCAGAGTTCACAATAGGATAAGGAAGATCATGTGTAGAAGGAAGAGTGTTTGTCCTTCTGGTTAGT
GTTTAAATGTCTGTGCCATTACAGGAGAAAATAGAAAAGAAGACTGTGAGCCTTAAAAAGCCAACAGCACACACATGTA
AAGTTGGGCAGGGGTTGGGGGAGGGGGCTGGCTCAGTTGGTAAACTACTAACCTTGCCACAGAGGTCCTAAGTTCAATC
CTCAGGACCTTTGTAAAAAAGCTGAGCAAGGTGCTGACCTTTGTAATCCCTGAACTGAAGAGATAGGGATGGGGGGGGG
GGGAGGTAAGAAAGAAAAGAAAAAAATTATCCTCTGACCTCCACATGCTTGCCCACAAGCAAGGGCACACACATGTACC
TGCACATAAATACACATAATGTACAGAGAGAGAGAGAGAGAGAGAGAGAGAGAGAGAGAGAGAGTTCTCAGAGGTAATA
AAAGTTCAACTAAAAATATGTTCCATGCATTAATTATTCTAATGCTAAGAGCTTATGTAATGACAGCATTCAGTCGGGG
GTGACAGGTGACCAGGGGTGGACATACAAGGCTAGTGAGTTTGTAAGTGGACACAAACTTTTTTGGAAAGTCATTTGAC
ATTGTATATATCTCAAGCCTTAAAATAGTCATAACCTTTTGAGTGAGTGATCCCACTTCCAGGAATCTATTCTAAGGAA
ATAACCAGCTAATGTGGCCAAAGATGTAGGCACACAAACATTTACTGTGGCATCACCCTGGGGAGAGGATACTATGAAA
CAAGCTAAACTCCTACCACTGGGCAAAGGGGAAGCAGGTGTAATCACTGATTTGCCCTTTAAATTAGTTTTGAGGAGGT
TCACTGTGTGTTCCGAAAGAGGCAAAACCATGACACCAACCTGTACACGGTGCCACCCTACTGGCATAAGGGGCGCACA
CTCAACTATATGCAGCAACAATAGGAGAGGAAATGCTAGTGAACAGGACACTGAAAAGTGCTCACACCTAAATGACAGG
GTTCGGGTGATACAGTTTCTCTCTTGCCTGGTCTTCTGAATTTTCAGTACTGGTCATGAGTTTGGCAGAACTCATGGAC
TTTCGTGTGCTGGAAGATCATCATGACCTCGCTCATCCAAAGAGGTCACATTGCTCTGAAGACTCGGTCTGGTCACCTT
CCCTGTATCCCCTCGCTTCCTGTAGCCCTTCCCTAATGCATCCATGGAGGAAAATACGCTGTACCAGCAGGCTCCCTGC
TAAAAGCTTTCTCTGTACAGACTGGTGCCTGATGCCTGTTTCTGTGGGGGAAGAGAGAACTTTGCCTCTTGGAAACCCT
AGGGAAGAAAAAGGGTTCAAACTGGAAGCCCCAGAGAGCTCCTCTTTCTAAGGACAAACAGCAGTGTGAGGTAGGACAC
AGGAGAACACTCCTCTCCTGTGGGAATCTATAGCCAGGAGCAAGTTCCTCCCCAAACCCCCACTCCCACCCCTACTTCA
CCTCCAAAACAGCAGCCCCAGCTCATGGCCTAGAATTCAACAGACAGCTGACAAGTCCACCTTCAAGCAAAGGAGTGGA
AGCGGTGAGCCTAACAGCTGGACGTGCCTTCGTGCCAAATTGGGAAGAGGAGGCTGATAACAAAGCAACAGTCATGCAT
TGTCTGTCTAGAGCGCTGTGGTTTGAGGAACACATTATTAAGTGATTTCATTGGTGTGTGACCATCTCACAGTGTATTT
AACAAGCTGGAGCAAGCATAACAAGGCTTCAAAACATTTCCCTCATGTGACCTCTTTTCATCCCCCCAAAAAGTTGTAT
TCAATCACAGGTATATAAGTATGTAAAATAATACAAATGAAGCATTTACAGTCATTGCTATATCTATAGCTTTGGCTAT
TAATTGAGGATGAAGGTTAATTTGCATACTGATAACATGATTGCTATCTATTTCCACATAAAGAATTAAAGTGTGCCTG
AGTATTGGATAGTCTATCTTCAATGGTTTTCTCCGAGTTGACCAGTGTTTGGATTTTATAATCATCAATCCTGAGATCA
TGGCTTCACTTAAACAGGAAGGACCAAATAACAGAAGAATATATAGGGTTATTGTGAAATTGTTGGAAAATTGACCCTA
ATCCCAAACCTAAATTCATCGAATCATTTTTTAAACAAAACCCAGGTCAGCAACTTGAGAAGCAATTTTTAAAATCAAA
TATCCTGACAAAATATACTGGAAGGGTAAAGTAATTTGCTACTTATATCCTGAGGGATAATAGTAGGAAAATAGCTTTA
AAGTTTTTATTTTGCATTAATAAACTGTTATGTTTCTATATGTGCTGAAAATTCTGTATGTGCGGTGAAATGCCACAAT
TCATAATGTACAATTGTCTAACGCTATGACTATTGTCTTGAACCTGAGCCCATATCTAGGGCAGCAGTGGTGATGCCTC
GTGAGTGATGTCATGCACAGTGCTAAGTGTGCGTGTTGTGTATTCGGAACCAAGGCTGCAGTGAAGTCCCACAATGCAC
TGAGCGTGAGCACCACCAGAAGTACTCAGACTAATTACGCATTGATTTTGAGTTAATCTTTGGTCATTGTTCATTTAGA
AACTCTTGTTATTGCCAAGTTTTTTCTACAACTCACACATTTGTGGGCTCCCAGCCCAGTTAAGAAGCTTTGATGTAGC
CTGCTACCTATTTCCTCTCTACGATATCAGTAAGTAGATATGCAAATACATGATAGATACCGAGGGGGACAAAGGTACG
TGGGTACGGATATAGAGTAGTCTGTTGCCTCTGGGTCATGATAAACAAATAAACAGGAGGTTGGGTCAAACCCAAGAGG
AAATGACACAATCAAAAAACACCATAATGGAGCAAAAATTTCTTGCTTTTTATAAAAGTATTTACATTAGCATATAAAC
TATAAGATAGCATTACGGCATTTTCAAAGTCAGATTTGTTTTATAGTGAGCATGTTTTTTATAAATACGAGTATAGTCT
AACAAGAAAAGTATAGTGTAAATACAGGAATGCAGTGTGCATAGTCACTATTATCAAGTGTGTACTTTGCACAAATATA
CTGCTATGGTTGCTGGCAGGTAATAGGCTTGCCTTCAGCGCATCAGCAAAAACATGTGTGTGCTGTGTAGTGTCATGGT
TGTTATAATGGAGACCATATCCCTAGGACAGAAGGAGTTTTCAGCCCCATCATAACCTTGTGAAACCTCCCTCGTACAT
GTGGTCCATCATTCTACGGAAACATTGCCTACACAATGCATGGTGAATTCTTACTGATTTGTGTCATTTAAATTCTGAA
ATTTCCAGTTCACTGTAAGTTAAATAATTAAGGATTTAGCCCAAAATGCAAGTAGTCTCTGTTTTGCCAGACCTAAGGC
AAGGGTAGAGTACAGATTAATTAGTGAGGTCAGAATAAAATCTTTGAATCCCATTATTCAACATGGTATCAGGATGCAG
GGGCTACCTCTTAGAAGAAAGTCTAAAAGCAGAAAGGTCAGGGGCGTTTGAAACTAAGAGATGGTTATGGCTGCAGCAC
AGGTGCACATGAGGCTAGTCCAGGGTGAACCTAGGCAGGGAACAGTTCTGCAGGCCACAGGAGCTGGGTCTTGGCACTA
AGGACCATAAGGAGGATGAGCCCAAGTGCCTGGCAGCATCAGGACTCAGAGTCAAGGTGACCCGAGTTCAGCTTTCTCC
AAAGCAAGACTCTCAGTGCAGTTCTGGAAACATGTGTTGACATCTAGATACAGTCGAGGTCATCGGGATTGGGAAGGAG
GTAATATCGAATCTGATAGGGTGCTGCTAAGTATCCTACAACACATAGGATGGCTTCCTACAGCAAAGACATCTCCGGC
TCCGATCTTCAGTCATAAGGATGGGAGGCAAGCACTTCTGCAGACACTTGGATTTCACACATGGACAGCAGGACCACAC
ACACATATACACGCTATCTGCCTCTCTCTGACTGATATACTTCAGCTAACCATGCTTCCCAGAGATGGCTCTAGGAGGG
GGCATTAAGCTGAGTCCTTAAGAATGATATTCTGTGGCATGACAGCAACGGTGGAAGGGACAGAAACATCTCTGTATGT
TATATCACGGATGCTATCTGCCCTTATATCTGCCCTTAGCCTGCATCTGTCACCGTTCTTGTCTCTGGTACAAAATACC
TGGCAGAGGCAGTCTAGAGAAAGAATGATTGATATCACCTCAGTTTCATGACCTGTTCCTGGGCACAGAGAGGGACCTC
ACCGAGAACTTGAGGTGGCTGAGGACATTAGGGTGGATCAGTCAGGGAGGGGAGGGGGGAACTGAAGAGAAGATTGGGG
ATTCTGAGCCTTTAGGCAGAGTGGCTACTCCACCTGCTCAAAGCCTGTCTCTAGTTGACCCCTCAAGCAGAGCACAGTA
CACAGAGGTAGGCATGGCTTCTGCCCATGGTGTGGAGAGTGTGGCAACTTTCTTACATCTCAGTGGCCAGCCAGGATGC
GCAGCTAGTTCCAACCAGTGATCCAACTAGGCCCTGCTTCCTGAAGGTTCCACAGCTTCCTCAAACAGCATCAACTGTG
TGTGTGGGGGTGGGGGGCAAGTCTCTTAACAATGGCCAGGTGGGGACATATCACATGCAAACCGTACCACTGTCTCTTG
AAGGACTTAGGCCAGTGGTTCTCAGCCTTCCTAGTACGGTGACCCTTTAGTACAATTTCTTGTGTTGTGGTGACCCCCC
AGCCATAAAATTACTTTTGTTGCTACTTCGTAAATGTAATTTTGCTACTGTTATGGACTGTAGTGTAAATATCTGATAT
GCAGGATATCTGATCTGATGTGTGGTTCCTGTGAAAGGGACCTTTGACTCCCCAACCCTAGGGGTCTAGACCTACAAGT
TGAGAACCACTGACTTAGAACACACTGGGTGAAGGAGGTGAGTGCAGGTCCTCATGGTTGCCTAAACCACCTCCAGGAC
CCAGCAGGTTTGTATAATCCAGTCCTTTCTTTCTCAGCCTCTGTGTGTGTGTGTGTGTGTGTGTGTGTGTGTGTGTGTG
TGTGTGTGTGTGTGTGTGTGTGTTGACACACATATAGTCAGGGAATAGGGCTCACTTTAGCAGAATGAAACCTATCTAG
TCCACCATCTGTCTCCCCTGTCCATCAAGAGACTCAGCTGTCTAGCTACACCAGTTCAACATTGTTAGAAGCCGACAGA
CATCCAGTTAGAGAAGACACAGTGGCGACCCAATGCTGAGACAATGGAGAGAAATGGTCCAAGGTCACATGGCCAGCAA
GTGGTGGCACTGCTGCTGGAACATAGCTTGTATCAGAGTGGCTGTTTCAAGGACAAGGTGGAGAGCTCTCAGGAGCCAT
GGACCAAGGTGGAGTCACAGAGCAAGCAAGGCCTCCTGCAGAGGTGGCATCTATTACCTGTGTCAACACAAATTAGGGA
ATTGTCTCCCACCTGGTTGAACCACAGTGCCAAAGGCTCCCAAGCAAAAGCCATGTAAACACTCCTTGGACCTACAGCC
AAATGCCCTGTCGCTGTTGGCTGGGCAGAGCAGGCCTGGCTAAAAATAGAAGGGGCTCTCAGGTCCAGCTCCCTGACAG
CAGCTGGCAGCTCCCAGTTCCTTTCTTTCTCTCTCTCTCTCTCTCTCTCTCTCTCTCTCTCTCTCTCTCTCTCTTTCTC
TCTCTCTCTCTCTCTCTTCCCCTCAGGCAAGAGCTAGTCTTCCCGAAGGAATGGGGAATTTTAAGACCTCCATCTCAGG
AAAAATAAGATTGTTAGCTTGATGCCCAGTACATATGACCCAGGGTCTGACACAGTCATCAATGACAGTTGGGGACTTC
ACCATAAAGCCTCAATCTACGAAAACTATCGACTTTCACAATCTACCCAAGGGAACAAAGAAGTCAAGGCTAAAGATGT
GGCCAACATCTGCTCACTCTGCAATTCAACCTTCTTGCCTTTCTTTTCCAAAACTATACCAGTTCTGATTTGAGGTTTC
AGGGTTGGCCAGTGACTTCTCTTAGGGGATAGATGGTGGCTATGAGGCAGCACTCAGCCCAGCCACAGAGAAAACTTTG
CTGGAGCCCAATACCTGCACTTCCGTTGTACAGGGAGGAAGGGTCATAAACCCTGCAAAGTGCATCCTGGGTGCAGTTC
AGGGCCGCGCCCGGGGATTTCCTTTCAAAGGTGACTTAGAGGCACTTCAGGAGGATAGCGCCCTCCCGGGCGGGTTCTA
ATGGTGAATTTGGCAGAGTCAGCGAAACTGCGCCCGGCGGACCTGGCGGCGGGGCAGCGCCCTCTCCCGCGCCCTCGCC
CTCTCCCGCGCCAGGCAGCAGCGCGCACGGGGCCATTTGCAGCCTAGGCAGCCTCGCGAGCAGCCGCTGCGCGCGGGCC
GGGAGGGAGGCGGCACCATGGAGCTGAAGAAGGACAGCAACGCTGTGGCCATCGACATGCTGCTCATCGTGCACTCCGA
GAAGCGGCGCGCGGCGCAAGCCACGCACTTGGACCCGCAGGCGAACCCGGGCGCGCTGCTGCAAAACCGAGGAGGTAAC
ACCCGCCTGGCCCGGGTCCTCAGAATCCCTGGGCACCTCGGCCACTAAGCCCCCGGGTCCTCTCGCCGGGGGGCGCCTA
AGCAGCACCCCCCTCCCCAACGAGGCTAATCCGAACCTCTCCGAACCTCTCCCAGACCGCTGCTTGTGCCCTGGCGCCA
GGGCACCTGCGTTCCTTGGCAACGCACAGCGCACTGGTTGCCCCAGGCCTGTTTTCTCCCGCTATTCTCTGAAGGGTGC
AAAACACATAGAAAAATGCTCTTCAGAAAGAAACCAGGGCCTTAGGCTGATCACGTGTAGCAGAGGCCTAGTCCCCAGT
CAGCATGCAACTCACCATAAGTTACCAGGGACCCCAAAGGGTCCCTGTACTGAGTCAGACTTTAAGATAACTTAGAGTC
TCAGTTTCCCCTTTCGTAAGCCTCTCTTTTCGCCTCCAACAGATATAAGGACTGAATGGGTGAAGGGAACCTGGGCTGG
GGTGGAGTAGTGTTTGAAGTCTCCTGACCGTACTTGAATTTAGCTTCAATTTTACTTCTTCTGCAGACTTTGGGAAAAC
GAAAACTGAGAAGTGATTGCTTATAGGGTGATTGCAGAAGGCAGAATCTGCCTCTATTCTACACACACACACACACACA
CACACACACACACAACTTGTATAGCGTATAAAGGGTGCCGAACGGACGCTCAGGAAATCACCGAAAAATGTAGGGGCCT
CGTGGCGCTGGTCTTGGAGGGAGCCTCTGAAGCTGACTTGAGGGTCTAACTCTGCACCACTTCTTTAGCAGAGATGCCT
GCCGGCTGCACAGAGAAAGATGACTGCTACCATCCCACTCTCCCTCCAGCTCCCACGTTTCTTGGGACCCCCTCCCGCT
GCTCTATCTGCCCCCCACTCCCAGCTTCTGTGCGCATGATGCACCCACAGCCTGTCCACCTGCTGTGCGAGGGCAGCGG
TTCTCTAGACAACCTTGATCCATCAGCCACATGCGCTGGAGCTATGATGCAGCCCCTGGGGAGCTGGAAGGGAGGCATT
AAGGGAAAGAGAGACTGGAGAACTGAGAGTGTTCGCAGCAGTTAGAGTTTAGGACGGAGTGTGAGGACGGGAGCTGTTT
AGGTTAATAGATCCTGTAGTTCTCCGATGCTGCTCCTGGGAGAAAGGATTAGACCCTCTCAGGGTCTTTGAAGGAGAAA
GAAAATGTCCTCCACGTCTTGATGGGAATGAGATGGCGCCCGTCGCATGTAACACAATCCAGAAGACCGCTCCCCGAGT
GTAGTGAGGCCTAGTTCCAAGTCAGCCCAATAGTCCCCACCGCCCAATCAGAGTCCCTTTGCTTCTGACACCAGAGAGG
ATAAAGAGAGAAGCCATAAAGTGCTGGCTATCAAAACTGCCCTCAAGGTACCCAAAGCCTACCCAAAATGTCCATGACT
CCAGAAGAGCATACAAACTTTGACCCTGCTTTACTTGGGGGGTAAAGCACCCCTACTTGAAAGGCACTGGTATTTTTCT
AGAAACATTTTCCCAGAGAAGGTTTCTTGAAGATGCTGGGCAGTAGAGAGAACCGTGTCCCCTATTCTTTACCAAAGTG
AAAAAGAACAGTGTAGGGTCTCCAAGGTCTGCCATCCATGGAACCCCCAGGCTGTACCGACCTGTGTAGCCACACTTTA
GGGCATAGCCTGAGGAAGAAAAGCATTAAGTCAAACTACTTGTTCAGACGTCTCACACAGATGCACAACAGCCCAAAAT
CGCCCTGCCGACTTTCAAGTCTCTTTCTGTCACCTGCAGGAGAGTTCTCCCCCTAAGGAACGGCCAAGGAAGGCCTTAA
CGCGCCCCCTCCCCACACCCATCAGAGAAGAATAAAGTTCAACTATTGTCACGAGGCAGACACGGTGGCCTTGTGCCAC
AGAGCAAGGAGGGAAGGAGTCAGAGGGACAGGACAAGAAGAGTCACCAGCAATTTAGGCTGCAGGGCTGGCCTGGTCTC
TCTTCTTTTCTCTAAGAGTCCAGCACGTTCCCAGCTATTTCCTTAGAACAGCGCTCCCCACAACACAGACTATGAAGAG
CAACAGACTCTGCAGAAAAGATCGGGGAGGGGGGGCTGAGGAAGATTTGGGGGCTTCTGGGGTGAAGGGTTAGAGAGCC
AGACCTGAGACTGAGCTTCCTGGGAACACTGCTATCAAGTGTGCCACAGAGCTGGTCTTGTGGTGGCGCCTCTGCTCCC
ACTGCTCCCACTGCCTGAGCATACATCAGGTTGCATGCCAGGGACTCAATCTCTCAACTATCACTTCCAGCCACTGCCA
GGGAGAGAGCTTCCCTTCTCTTCTGCATTACATCATTGCCAGAGCGAAACATGGGTGCATCTGATGGGTCATGCTGAGG
CCTACACAGGTCCATACTCTAGCTGCAAAGGATGCTGGGAAAGTAGGCAACTGTGGCTCTCCACTCCCATAGGAACAGG
CAATCTCTGTATGCTTTGTCCCTGCATACAGAAATAGGCAGGCAAAGCAGAAAGAAAACTCTCAGAAAATAATACTTTC
ATACTTACATATTGGAGATGTGGAAATGGGTATTGGGCATCTGTATAGCAAAAATTCCTTCTGGAAAATCCTGATAGCC
AATCCCACCATCACTAAAAACTACATTTTTTTTTAAAATTTAAGGATTGCATTGTTTCCTCAAATGTGTCTCATTCAAG
GGCAGCCTATCTCAGATCCTGAAAAAACTGGTTCCCACAGCCTTGTTTAGCACAGATGGGTGTCTAAGTGGAACAGTAG
TGCATGGTACTCGCCCTAGGGCTCCCTCCACAAAAAGTCTCACACCCAATCTTCTCTCACAGGATTCCAAGGTGTCAGA
AACGGAATCCGGAAGTGGCAAGAGTTGGAAGAAAACAGCTTTCAGGGTAAATCTCTTTTTCTTTCTATTTTCTGTTCCG
TCTCTGTGATTCAAAAACCCCGAAAAGTGGCTGGAAGAGTTGGGTTGTCCTCATTGGGAAATTGGCCCTAACATCTAAT
CGGTGAGCAGACATGGCTGCCTGTGGCTCGGCCAAGCCTCTAGGCCTCTGTGAGCCTGGAAAACTTTCCCTGGCATCGG
GGCTCCTGTGTTTCTGGAAGCTGAAAGTGGGGAATGATGCTGTGAACTTCAGCACCCATTTGTTTGCTGGCTGAGCTAT
ATACGAGGATTTGGCTTTACTTGGGGTATCGAGGGTTGTGTGTGTTCCCACCAGAGGCAGGGCTGCTGACATCACAAAG
ACTATATACAGCTCTAAAATGTGTTTGAGTTTTTTGTTGTTGTTGTTGTGTTTTGTTTTTCTGGTCAGACTGGGATAGT
GGCCTGTCTTAAATCTATATTACTTGAGGACTGGGCAGAGCCGATCAAAGGTTGTATAGTAGTCCCTACTGCCCTGAGC
TTCCGAATACAGCTAGAAGTTCATTTCCAAGTTCATTTCCAAAACCTCTTCTTTCTGGTTCCAGACAGCGCTCTTTTCT
GGAACACAGGCGATGGGCCAAAATAGCGGGTTTTGTTTTGTGTGCCTCAAAGACTGATTACCTCAGCTTCCAGGGGGAT
GCATGAGCGTTAACATGCATGATGAGAACACAGACTTCTACTAGGACCCCGACCTCCACTCGCAGAATGTTGCTGAGGA
GTTCCAGCAGACCCCACCCATGTAACCCTGGAGTCAGATGTCTGCCCCTGCACAAGAGCACAGCTGAGGAAAGGTGAAT
AACTCCGCCTTCGCCAGCCTTCAGACAGCGGCAAAACGGAAACGCAAACCAGGAAAACTGATCTTACCTCTGTGCTTAT
TCCTTCAACAGCTCCGTTTCGCAATTCCTTTGCGCCAGGCCAAAGTTTGTTAGAGATTGAACAGTTAAACAAGCAAGTC
TAGAACCCTGTCCTGGTAGAGCTGTGTTCAAATGATGGTCATTTCCCCACGTCCCTTATGCTGTTCACGGGAAGGAGTC
ATCTTCAAGATGCAAGCACACCCATTAAGAACATCCGCCTGTGTCCTGGCTAGTGGATCCGTGTAAAGGCTTCCTTAAT
TGGGGCTAAGCTATAAGGAAAATCTAACTCACTTGGGGTGGCAAAGATTGACATTTTAGCTAGTGAAGCGATACTCTAG
GCCAATAAGAAGCCTAAGAATGAGAGTCCAGGAACTCCAGCCCTAGGAATGGCCACTCAGAGCCTGTGTGCTGTGTGCT
GGGTCCATGTGTGAGCAAGAAAGTGAGTCAAGTGGGAACACTGGACAAAAAGAGAATATCCTTTATGTTGCTTTTCTCT
CTTGCCTGTGTGAGCCATCTGTCAAGCACCAAAGGCCTCTCAGAAAACCCTGGAGTCATTCCACTTTGGGCATCTTCAA
GGTTGGAATGGAGATTAGTGAAAAACTTCACAAAACAGAAAGCAATGCAGTAACTCTCAAGATGATTATTTAAAAAAAA
AAAAAAAAAAACAACCCTCTGAGGCTCTTAGTATCCATCCACAGAAGGTGATTTAAATAGTCCATGAAAAAAATCTAGC
GTTTGAAGATTTGGTATGACTCTGGCAGCAGAGAATGTCTGTAGAAACCTTAAATCCAGGAAACATGATGGTTTCGCCA
TCCCACCAACATGTGTAATTTTAATGTAAGATTTGGTCTAGAAACCAGGCAGTGGTGGCACATGTCTTTAATCCCAGCA
CTTGAGAGGCAGAGGAGGACGGATTTCTGAGTTCAAGACCAGCCTGGTCCACAGAATGAGTTCCAGGACAGCCAAGGCT
ACACAGAGAAACATTGGCTCCAAAAAAAAAAAAAAAAAAAAAAAAAAAAAAAAAAAAAAAACCACACAAACAAACAAAC
AAAAGACTCAGTCTACTTTTTAAATGACATGCACATATGGAGAAATATAGCAGCCTCTTTCCACTTTTTTTGAGGGCAA
GCTCCAGGGCACTCGCGTTGAAGCTGATTTCATCACATATGTGCCTTGCTGGTACCCAAGTCACCGAGTCCTGCTAACC
AAAAGGCTGTCCAGACCCGGTAGTTGGAGAAATTTCATCTCAGAAGGAAGTGGCCCAGTGGCATGTGCTGACAGAGTGT
GCACAAATGGAACCAAGTGTGGTGTTGATCCAAAAGGGCACATTTGTCCTCCTATTGCTACTCTGTACCCTCCTTGGGC
TTGGGTGTTGGCGAGAGGTAGGGTGGGGAGCCTGAAACACTCCAAACTTTTGGAAGCAGCAGGTGTTTGGGCTCAGCCA
CCCCTCTCCCAAAGCTCGCCCAGCAACGGGCTTACCCTGCTGTGATTGGCTGCTCTCCTTGGTCCTGGCTGAAGTCTGC
CTAGCAGCACAGTGTCTGGTGCCCTACCGCTAGAGAGAAACCACTTGTGCAGCCCTGACTATAAATACCTCCCAGGAGC
TGTGCCAGCCAATCATCCTTCTTAAGGATTCCCTTCCCGGCTCCTGTTTCCTCATGGAGGTACAGCCAGCCGTGCCCAC
AGAAGGCAGCCTCCCTGTCCTCACCCGAGTGGCCAGGAGCTCCAGCCCAGCATGCCACATCACCCCACATTGGCATACG
ATGACAGTGCATCTGGCCTTGGCTAGGCAACCATGTTCTAGAAGTCCTGGGGGCCTGACTCAACCCGATCCTAGGAGAA
GACTAGAGCATGCCGGTCCCCCAAGGCAAACCTCTGCTGGGTGGACCCCAAGTCTAGCCCAGCTGAGTGCCCCACTTAT
CCACCTTCACCAGCTTCCGCTAGGGAAGCATCGAAGCTGTGTCATGGGAAGCACAGAGTTGCCTTCGTGATTGGCTCCA
CTCTACAGACAGCAACCTTAGGGAAATGCTAGTTCCCAGGCTGAACCACTGTTTTCCCCTAAAGTGAGGCTCACTCTAG
GTCACAGGGAGATGACGAATTATAAGGGGAAATATAGACTGAGAACTTCCTTCTGCCATAATCCTGGTCCTTCCTCGAC
TCTTCCCACATCCATGAAGACAAGGTCCCAGGGCTGGCTCAGTAAGGACAGTGCTGGCACAGCCACAGAGCAGCCATAA
AGCAGAAAGGAATTCATGCCTAGGAAGACCCGAGAAGACAGCGATGGCCTGTAGCAAGAACCAGGCATCTCTCTTCAGC
ATCCCAACCCCACTGGGCCATAAGGACATGTAAATATCACCAGTGGACACAGAGAACTCAAACATGTTGAGTGCTCTGT
AAAAATGGTGAAACTAAACCAAACTGAGTATCCTCCCACCTTCAAGGATAGAAGCAGAGGCAGGTGACTGTCCCTGTGC
TCCTCAGAGGGAGGAAAGAAAGCTGAGCTAGGAGTTTGGAAGCTCAAGAGTTCAAGTACTATGTTGGACCCCCACCCTC
ACCCCCACCCCCACCCCCGCACAGTTCACTCCACAGAAAGGGATGATGACAATCGGTTAGGCATCTGTGTCTGAGAAAA
TATTTTGGAAATTGTAGCAAAAACTCCAGAGCAGCAGTTCCATAGACCAGTAGTATGAATCCCGGCCTGGGTAAGCTTC
CACTGGGAGGCAGAAGGGGTGGGCACTTGTGGACTCCCATGGGTGAGGCTATGCTGGTCCAGAGGTGTGCTCACTATGG
CAGTAGATGTGCTCAGCCCCCGCCAACATACAATTATCTCAGGTGCACCCGGCTCCCTCAGCCCCCCTCCCAGGGAACA
TTATACCATGGTCATGGTGGTCACTTGCCATCGTCTTTGTCTTTCTTAAATAGGAAACTTGCCAGAGAAGCAGTGCCTT
CAGCAGCCGCAGGTCATCACCTCCTACGACAACCAAGGTAAGAGACAAATCGCTAATGGCCATCTCTCCTGGGGTTGTG
TTCCAGGCTCTGCTGCCCAAGTCAGCGAGCCCGGGATACAGAGATCAGGCTGCAGGGAGCACCTCTGCTCTGATCCATA
AAAGCTATAGGGAGCACCTTCTCTGTGATCCAAGAAGGCTACAGGGAATACCTCCTCTGTGATCCATGAAGGTTAGAGC
ATCCTTCTCCCTGCATCCCAGAGGAAGAACTGAGACACCCTGCCCTTCCAGTGAAGAAATAAAGTCCTTGGATTACAGA
GTCCCACCATTATCGATATTGTCAGTGCTACAGCTAACACTCACTGAGTGCAAAGCATGTGTCTGCGGCTCTACCAACC
TCTATACGCACTTAAGTATCAAAGCAACCTTGACTGGGTGAGATGAGGAGACTAAGGCTTGAGACAGCAAGAGTCAGGG
CTAGCTCTCTGGTACCTTACCTCCAACCCCAGCAGGGCCTCAGTTGTTAACCTTCCCGTGGCTGGGCTTCTGCGGCTTC
CTCGAAACTGATCTCCTGCTGGCTCCCAGTATCCAAGTGCTCCCACATCCTGAGTCACAAAGGCTCCTTGACCTCTCTT
GAAGCTGAACCTGATGAAACATCCCACTTAGCGCCAAGTTTTCGGCAGCAAGGTGGAACAACCTGGTCCTTCATTGAGA
GCTCTGATTTTCCCAGAAGTGCTCTGGGTACTTGGAAATGGCAGGCAAGGGCACTCTCTGCTGGCCAGGCTGATTCTGC
AGTTTTCTAGATCTGAGACTACCACGAGGTAGAGCTGAGGTTTCCACTGTGGCTGGAGGAGAACCTATTTAAAGGACTC
CAGCATCTACCATGACTCTTCGTCAGAAGCCTGGGGTGCTCAGGCTCAACCCAGAGTTGGCTGCCATTGCTGATGCCAT
AACAAGCAAGAAAACTATGCCATAGCACTCAAGGGACATGGGTATGCCCCTTCCTGAAGCACAAGAATCAATGCAGGCC
AGGCTGTTTCTCTGACACTGACGTGGCTGTACAAGTCTCACAGCAGAGATACCTCGCTGATGTAGAAAGATTGCGAATG
GGCTGCCATTCTCTCAACACAGGGGACAAAAGAAAGGCTCCTGCATGTAGGACTGGGTCCTAGGGAGCGGGAAAGACCG
AAGGGGAAATTAAGGCAGGAGGGATGGTGGGGGCAGGGGTGATAAATCAGTTTCCTGTTGCTCATTGGCTAGAAACAGC
AAGATTTCGCCTTAAGCTTCTGGGACTGAGAAGGCCGAAGTCAGCTTTGCTGGGTAAAGGGCAGGTGTCAGCGGGCCGC
AGCCTTTGAAGTCTTGGGAGGGAATCCATTCCTTACCTCCTCCTGCTCCTACAGGCTGCCAGCATTCCTTGGCTTTATG
GCTGTGTCATTCCAGTGACGGCTTCTATTATCACATGGCCCTCTCCCTTTTTAGGTAGGCAGTTCTCCGTCTCCCTCTT
ATAAAGTTATACGTGGTCACATCTAAGGCCCAACCAGATAGCCCAGAATAATCACTCTGAGATCCATAGGTAAGGCCCC
ATCTGCGAAATCTCCCTCGCAGTCAGAGATTCTGATGGATACCCTGAGAATTCATTACTCAGCTCACGATAAGGAACCA
TTGTAAGCAGAATAGAGGAGGCTGGGAGTGTAGCTTAGTTGTTTGGCTATCATGCATGAGGTTTTTTAGTCTGATCCCT
GGCACCCCCCACCCCCAGATAAAAAGAACAGGTGAGCAGCAACTGAGATGGAATATCTGTGTAAGAAAAGAGGGGGTGG
GACAGACACGAGGGCATCTGGGGAGAAATTTTAGCTGGGAGTCATGACATGAAGGCACAGGCAGGCAGGCAGGCATCTG
CTAGGTGTGGAAAGTCTGTGTCTGTGGCCAGGGGAAAAGGGGACATGAGCAGAGTAAGAAGCAGGAGATGAGGCAGAAA
GGATTCGAGGCAGGACTTATGGTCCTAGGTGTGGGCTGCCTTCAGTGTGCTGGCTTCTGATCAGAGTATGATCAGCAGA
CATCAGATGGCTTTAGATGTAGTGCCTCTAATATTGGAAGAATGAACAGAACAGAAGGGGGTGACTAGAAGCAAAGAGG
ACTCCCCCTAGAGGTTGTATCCAATGGCAAGAGGGTTTCCCATGTTGTGGATCTGGGCTAGAAGGAGAAGGGGGAACGG
GGAGACAAGAATTGAAGCATAAGCCAGGGGTAAGACACAGCAGACTGATCAGGGGGCGGGGGTGGGGTGAAGGGGAAGA
GTTGAATGTCGTTCTGTTGAGACTAGGCATTGGGGAAACAGAAAGGATGTCCCCATCGTGTGTTGAGTTTCTACAAATC
GGCAAGCCTGGGACTCCTTGGGCTCTGTTCTAGGGACCCTGTCAGCTCCAGCACAGGAGGTCCAATCATAGCCTAGATC
AGAGGCAGAACACGAGGTGTGTGCCAGACATCGAGCTAAGCACTGTTCATGGCTCCTCATCTCCCTCAGCCACACTCTA
TGAAAGGTTTTCTTTCTCACACACACACTAAAAAAATGAAATCCACTTTATGGATGGAAAAAAATGGAGAAACCGAGGC
ACAGGGGCATCTAAGCAGCTTACCTGAAGTTATGCACTAAACAGGACAAGTCAGATCCAAGAGTTGAACATGTCTGTGC
ACCCCCTTAAAGCCAAGTGGGCTTAACTTCTGTACCACACTTCCCCCATACAGGACAGGGACCACAGAGCTGATTTGAG
TACAACGTCCCTGCCCACCCCAGCAAAAAGTCCAAGGCTCCAGATGGAGTTTCTTATTAAAAATGGCTCTGCTTTCCCC
TTCTCCTCAGTGGCAGGTTTTATACAGCAGGCAATAAAAAGCCTGCTCGTAGTGCACAGGGGCCTTAGAAGATGAATCT
TCAGTGGGCTTGTGTTTGGGTGACAGGAGAGTAGTCACCACAAGGAGGCACGATGGCTTCTGACCCTCAGGGTGGCGGC
TAAGGAGACAGTTCTGAAATGACACCACCCATCATGCGTGTCCCCAGCTTTTGGAGCTGACTGCTTCCCGACTACTACA
TCCTGGAAGCATAGAGCACGCATCTTTTAAGATCTAGCCAGCCATGGTTCATTTGAGAGTGCTTTTAAGATGACGAATG
TTTAACTAAGATTTTTCCGACTTTAAGTGTTATGGAAAAGTATGCCCAGGTTGTAAATGGATCAGCATGTACAGAGACT
CTTAAGTCAATGGTGCTGAGTATCTTGCATAAATCCTGGGACTCCTCCGCATGCACCCCTGTCACCACCCCCATGCATG
CATACACACCATGCACACATGCTTGCCTGGCGGTCTTTGTTAATTGATCAATGCACCTGGCAGCCACTTCCAATCAGTC
AGAGTTAATGAACAACACAAAACCCACTTGCCGTGCCCACGTTAGATATTCTGAGTCAATTGAGCCGTTAGACAAATAC
CGACTGAAACCTGGAGCCATTAGGAGAGCTGTCGTGTGGGAGATGTCACCTGACTGGGTAAAAGGCCAGCCTAGAACAG
TGCCCACACAGACTCGGGATGCTCTATGCATCTCAAATTTTGAGAGAGAAAGGGAGAAAAGTCAGCAGGGTTTAGAAAA
TGTCCAGCCTGAAAGCCACCTCATAGGGGTAGCAAGAAATATCCCTAAATGGCCAAGGCTGCAGTTTCAGAAGTCTTAT
TCAGCAAATGAATTGCTGGGGAAGGGTACTATGAGAGAGGCCAGGCAGGACACCCAAAGTCCCAGAAAGGGTCTCTTAT
CCTTGACGGTCAAGGTTGGTGTGATTGGCAGCTGCTTGGGATTTAGCTCTGACTTGAATTTTGCCTGAGATTCTTCGTG
TTGAGAGATAGGTCAGGTTAATATTTTTACTAAAATCTCAGTTTTATTAGATCTAAAAGTGAGGTGGGTACGCTGTCTG
CCTCTCTAGTGAATGCTAACCATCTTGACACAGGGTGATGACGGCAGGCCATTCCTAGGGGGCAGAGAAGTCTCACAGA
TGGTCTCTTCAGTCTCTGGAGGAATGCAAAACTCCATAATCAGGGTGAGAGTTTGACAGCGTTGAGAATAAAAACAGCA
GCACAGCCTAGGGACAGTTGGTCCTGGGCAAGGAGATAAAGTGAAAAATTGCAGGGAGAAGAGGGGGTTCAAATGATGT
CAGGGAGAGCTGGTGAGCGACAGCCCCTCTAAACATGAGGCCACCTCCCCGTTAACCCCAGAAGGGTCACAACTGTGGT
TTGTTTCCCAGCCCCACCACTCTTCACAAGAAACATGGTTGGAGGAGGGCAGCTAGATGTACATAAGCTAGGAGAAGTG
CGAGGGAGATGCTTACTGTTGGACCCTAGGCCCACGAGCTGTTCACCTTCGCAGCTCCTTTAAGGCTACAGTTGGTTCA
GCCCAGTAATCTCCAAACAACAGCTGCACTGGGTCAGACAGACTCCTGGGTCGGATAGACCCCTTTGCTGCGGGACTGA
AAGCATTGCGGGTTGCAACCCACAATAGAGAGAGCGTGCAGGGGAGCCTCTCACAGTTCATCTTTCAAAAAGCATCTTG
TGGCCACACACCACTTTGGAGAACACCTCAGAAAGAGTTGCACAAGAAAACAAAGAGGCCAAGTTAACCAGGGCCGCAA
AGGGAATATGCTTCGGATGAGGGTGGCACCCAATCTTCTGACAAGAATGGACTCACACCCTGAATTCTGAAATCCTCGG
CATCTTCATCCCAGGATCACAGGGCTGGTGTCATGGGCAGTGAATGTGCTAGATTCTACACTTTACAGGTGAATGCTGG
GCTACAGAGATAGCTCAGTTTTAAGAGCATTTGCTCCACAATCGTGAAGACTAGAGTTCGAACCTCAGCACCCACATGA
CAGGGCAGGCATCCTCTGACATGCCTTTAACCCAGCTCAGAGGCTGTCAGAGATTGGAGGCTCTTGGACTTGCTGGTTT
CCAGCCTAGCTGAAACACAAGCCTCGGGTTCAGGGAGAGACTCTGCCTCAAAGAAATAGGCGGAGAGTGATAGAGATCA
CCTTATTTATTGCATGTGAACAATACATATATACCCCCCACACACACACACACACATTAACTAATTTATTAGTTAATTA
AATATTTAAAATGTTTGCTAAGCCTTTTGGTCTGGGGAACACACATTGTATCTCCAGTCTCGTTAGCATGCAAAACAAG
TTTTAAAAGCGTATAAGAAAATCAATAATGGTTTTCCAAAAGAAAATAATTGCAGTATATTTTAACAAAGGATTAATAA
AAAGGAACTCATTCTTACAAAACAAAGGAAACATAATAAATCAGTAAATAATAATAACAATCAGCACTCAAAACAATTT
AAAGAGCAAATAAGAAATTTTAAATCTACACTAAAATGTTTTTAAATACATAAAACAATAAGGGACCCTTCATTAATAA
AAATGTAAAGGGTTTATGAAGGCAGGTGCCAGTAAGCTTAGCAGGCATGTTTGTGTTATCCACCACCACGGGACTCAGC
TCCTAATGCCATCGTGAGCTTCCAAGCCTGCAGATCTGCTGGACGGTCACTGTCCACGCCATAGCCGGCTGATCTCAGC
TCTTGGCTGAGCTAGTGCTGGCTGTCAGCTGGTGGGCTGGCTGGAGACCAGAGGGTTTCAGATAGCCTCACCTGAGCAG
CCCTGCGCTGCTCCCTGCGGTCTCTCACCCTTAGTCTGGTGCACACACAGCACAGCACTGTGGCAGGGATGCCAAGAGT
GGGAGCGAGCGTTGCAAAGTCTCTCCAGGGCTAAACTCAGACACTGAATGATAACACAAGTTCTACCCCATGCTAATCA
GCCATACTAACTTGTGAGGCCAGCCTACATTCAAGGGGTGAAACAAAACACCTCTCGTTGGGAAGAAGATGAAGTGTAG
GCAATCAACCAGGGCAAGAATGAGTAAAAAGGGCATTCTCCACCTCTGACCCCATTGGTGGAATCAGAAACCAACAGAA
GGATGTGTGGGAAAGTCGGCCAGTATTACAAAAGCCTTCCAAAACCCTCATTCCTTTCCATCAGTGTTTAGGAATTCAC
TCTCAGGAATTGATTGGAAAAGTGTTAGAGATGTCTTCTTTATCCTGATGTTCACCACATCATGGTTTATCACAGCAAG
CACCTGGGGAGTGCGGAGTATCCGATGCACACAGCAAGTTGTAGTGAGTACTCTGAACACGTGACGTAAATGTGTGTTC
CACTGCCTTCCAGGACTGGGGGACATTCTCTACAGCACCGCAGAAGCCACGGCAGCTGACAGCACTATTTAATGCTTCT
CCGCAGTTGCTGATTTTTTTAAATTAATCCCTTCAATCTCTGAAAGAAGAGAGCTGATATCTCTGACTATAATGTATAT
TTGTCTATTCCTCTCTATAATTCTGTGAGTTTTATTTCAGATATTTTAAATCTTTGTGTTCAGGTGACTACGTATTTGA
CATTGTCACATCTCCTTGATAAGCTGACGTTTCTTCACTATTATATTATACTATTATGTTATATTTCTCTATCTCTGGT
ATTTTCCTTTCTTGAGTGGATATGTTAATGAGCAAATTGTCTTGGTCTCACTGTACCTGAAAAAATGTTTTTATTTCAT
TCTCATGTGGAATTTTTATTGGATAGAGTCAGTTAATCCCTTTCAGAATCTTAAAGATTCCACATCTTTCTGTCCTAGT
TTCTGATGAGAAATATGTAGCCACCTTATATATAATATATCACTTATCCAGAGCGTATTCAGGGACATTGTATCTCAGT
GTTGTAATTATGTCATGTCTGGACCTGGATCTTCTTTTATTTATCATGGTTGGCACACACTAAACTTGAGTCCATTCAT
GTATGTCTTTTCTCAAATTTGAGTAGTTTGTTATTTCTTCAGCTACTTTTCTGCACACATGGTTGCCGGAGACATGAAC
AGTGGACTTCTAAATGTGGTCCTGTGGTCTCTGAAGCATTGCTGGCTTTTCCCATTGTCCAGATTGGATCCCGGCCGCT
GTTCTCATGTTCTCAGGCTCTTTCCTTTGTCATAGAAAACATTGTAGCCCATCTTCCATTTCAGGGTTAACATCTCCAC
TTTGTTCTTCCATCTTCTGTTTCTTCGCTGTGTTTTGTATCTTTTGCTTTATTCTGAAATGGCTGGCCTTTCGTTTTGG
AGCATGGCTAATGTGCCATTTTATCACCTTTGGGTTAAATGTGGGTTGTCATGGTTGGCATCTTTTCTTCTCCAACTGA
GATCTTCCCAGGTACTCTGCTGAGCAGTTCTGAGTGGTGGCCTTTGCGGGGCCCATCTGCTTGCGTGCACCAGGAAGTG
GCCAGTTCAGAAGGAAGACATGGTTTTTCCCCTAGAGTTTGGTAGTCAGACTGTGGTATAAAGTGCATGGGGTGAGACG
TGTGGTGACCATGGGAACTCATTTGCAACTTTAGGGAATTACATTTCGTCAGCCATTTCCTGTTCCTCTGGCCAAAAAA
AAAAGGTTGAAACTTTGTCTTTTCACTCTGTTGGGTACTTTTTATAACTGTGTCTGGGGACAATCAGCAGGAGAGATGA
AGGAGGGAGGAAGGGAGGATAGAGGGAGGGAGAAGAGAGGAGAGGGAAAGAGAGGGAAGGAGAGAGATGGTGGAGGGAG
GGATGGAGCGAGGGAGGAAGGGAGGGAGGGTGGAGGGAGGGAGGGAGGGAGGGAAAGGAGACAGGGAGAGAAAAATACA
CATGCCACTTGTGCCTGGCTACTAACCTCACAGCTACCCACATTCTTGCTTCTGGGCTGAATCAGGAAAATATAGAATA
AAGAAAAAAAAAAAAAAACACAGCAGAAGATGTCCCTGAGACAATTAGGGGTTCCCATTCTCACCCTTCAAGTAGAAAA
TGATTTTTTTTTTCCTTGAGTACTCTTTTACATCCATGCACCTCTGCGTAGGGATGCCTTTGCCTGAGGTGTTGCCAGA
ATGAGAAGAAGGGTAGACCGATGCGGTCCTGTCTCACAACCCGGTCGTTGCCACTCCCAACCCTGTTCTTTTCTATCAC
TGAATTTCCAGGGTCTGCAGATGGGTTTCCCCATGCATTCCACCCCCATGGTTAGATGGGGTAGAGAGAGCGCTTGTGT
TTGTTGTTTGGGTTAGTCTGGAAAACATGAATTATCTGCATATTCAACAGAAAGATCTAAGATGAAGCACGTTCTGCCA
TTTGAACATCTGTCACAAGACGGAGTGCCCTTCCTAGCAGCATTTGGCTAAGAATGTCACCTCCCCCTTTGCAGGTACT
CAGCTGACTGTGGAAATCCACCCCCAAGACGCCATGCCCCAGCTGCTTAAGAAGTTCTCTTTGGCTAAACGTACGTACC
TGTACAGGGCTGCTTTGATCGTCCAACCTGGGAGAAGGCAGAAACATTACTATTTACACCCATTGTGTTAGTTTCTATT
CTTATTGCTCCCATGGCTTTGGGAGATACAGTATACTGTGACAGAAAAAGTTTGGCAGCAGGAAGTTCCGTAATTGCAG
GAGTATGCAGCTGGACGTATCGCTGCCCCACATAGTGGTAAAACAGGAAGCAGAAAATGAACAGGAAGTGGAACTGGGC
TTTAGAGACTCAAGACCTGCCCACCAGTTACCCACTTCCTCCAGAGAGGCTTCACCCCCTAAAGGTTGTACATCCCTCC
AAGACAGCATTAATATCCAGGGACTGGGTGTCCACACACATAAGTTTATGGGAGCATTTCACAGTCAAGTACAACAGCC
AGATGCTACTGTTCTCGGGTCTCTGTAAGGAGGTCCGGGTGATGCGCATAACTAATAGTGCATTCTCTCACCAGACTGT
CACTACCAGGGTTTTAAGATGCCATCTGTAGGCTGCCAAAAACCGTGGTATGCACAGTGCTAAGTACTTCCCATGCCTC
ACCTTCTTGACCTTTATGCAGATGCTGGTATTCGCCCCATGTTTTTTCAAGTCAGCAAAGCGAGGCTCAGAAAAGAGAA
ACGACTGTCCAGGAGAGTTACTCGGCCAAAAGATAAAGACCCAGGTCCAACTCCGAAGCCTCCGATTTCAGCTATAATA
TGTGGGAGGTGTCAGTCTATGTAGGCAGGCAGGATCTTGACTGCAATGACTCTTCATCCCTGCCCCATCTTCACCCTGG
CCTGCCTCTTGGATGAAGGAGGCCTCATGAGGAAAGAGGCAGAGGAGGCTTGAGGCCCCTCCCGCTGTGTCCTCTCAAT
GATGCAAAACCCTCCAAGGCTATATCCTTGCAGAGCACACCACGACCCTTTTTTTTTTCACATAGAAAAGGAGGAAAAA
TAAGTAAGATACCCTTGTAGGATTGTTCTAAACTCCCATAAACACTAACTTCTCGGAACCCTGCCATTCCGTCTGAAGA
TGGAGGAGGAGGGGAAGAAAAGTTCCAAAGAAAAACAGAACCTATTTCTGGGAATGGAAACCACCCTCTCAGCTTTCAG
TTCCTATATTTACAGCCTGAAGTGGCTCTGGGGGCATAAACCGTCGGCCTCGGTGAGCCCACCCTCAATTCTGAATGGT
TTCATTAGACACCATGTCTTCCTGACCTGACTTTGAACCAGCAGCTAAGTCTCCTTTGCTTCTTTCCCAGGGACTCCCC
TGTTACTCACCATTCACAGTGCAGGGCCACCCACTCCAAGGGCTACATGACCTTCTGGGCCCCAAGTTCCATACACATT
ACCTTTTGTGCCCCTATTATCGCTCTTCGCTTAGTCAGTTCTTGTCTTGGCTAGACATAAAACTGCCTACATGTACTGA
CAACTGCCACGTATTATAGCTAGAACCAAAGGGTTTGGAGCTGTACTTGGGCCTTATTCTCTTGGCCAGGTAACTTTCT
TGGACAATCATTTCTCCTACCTGAGCCTCACTTTGCTGACTTGGAAAACACAGGGTGAATACAGGTCAAAAGGCTAAGG
CACAGAAAGTACTAGAAGAAACTGGTTTGTACCTATAGCGTCTGCAGCTATTTTCCAAGATGGCAGAATGCAGAGGAGG
GGTGAACCACAGCTCTGTGCAGGCCTGCTGCAGGGGATAAAGCCTTGAGATCACAGCATGCTCTGACTCAACTCAAAGA
CCATAGCTCCCTTTCCTGTCATTCATCCCCTATTCCCAGTCTCTTCTCTGGCCCAGCAGCCACAGTGCATGGTGGGTGG
GGATGGGAGCAGGGAGTCTGTAAGAGCCAGCATGAACAGGGAGAGTGGCCAGGACTACTACCTAGAAAATCGCCAGTGC
TTGCTTTTCTGTTTCTTCTGTGGTATCTCTGACCTTTGGGATATTCCAGGTTTACAAGGTGATAAAAATGGAAACATGA
GACCCCGGCAGCCTGGAGGGAAAGATGCCCACGGTGAGTCCCAGGCCTTCTCTGGGGCCTCTTTAAAAAAAAAAAAAAG
CAGTGAGTTGGCCTCATGGTCCAGTCTCCAGCTGCCAATTGGTACTGGCTTGCTGGGACATCAAGAGTCTGAGACCATC
TGTGGATCTGGGAGAAAGAGTGTCCTGATTGACTAGTAGTGTTGGCCACAGACACAGGTGAAGAGGGAAAACTCTTATA
TCATCCCAGCGCCAAAGAGTAACCCCTTTCTGAGTACAAAACAGTCTACTACAGAAAATGGTGGACCCGGGATGCAGAG
TGATGCTAATTTGGAGCTGGAGACGTAGCTCTGCTGGTAGGGTACCTACCTACCTAGCAGGCATGAAGCTCTGGGACCA
GTTCTCAGAACCACATCAACTGGGTATGCCAACATACTCCTGCAATCCTGGTACTCAAGAGGTGGAGGCAGGAGGATCA
GAATTTCAAAGTCACCCTGTGCTCTATGGAGAAAACCAGCCTCAACTGGAGACCCTATCTTTAAAAAGAAAAGAAAAGG
AAAAAAAACAGTAAAAACATAGATGAGGGACTTGGAATACAGCCCACTGGCAGAACACTTGCCCACCATGTGCAGGGGG
TCAATCCCCATTGTGACAAAAAATACAAAGCAATAAATAAAATGGAGTGTCAAGTGTTTACACGGTAGGAAGCTTGTGG
GTAGCAGGTTACGGCAGAGAAACACTAAATGGGGTGATATGAGGTGATAGATGGTCTGGATTGGAATCCTGCTTTTTCC
TCTAATTTGTCTTCTCTAAGCCTCAGTTTCCCCCTTGGTAGGCCGGATGCTAGCAGTGGCTCCGTGCACACCTGCCTAT
TTTATTTGGCCTCAAAATCAGAGTAAGTTAATTAATGTTCCTGGTGTTTTAAGTATCTTGCTATAAAATCATATTCTCC
TGTTTGACTCTTACATAATCTGCTTGGAAGGTAGATTTAACGAGGCATGAATCACCATGGGTATTTTGAATATAAAATA
TCAAATATTGTAAGAATGGAACCAGGGAAAGAGGAACTAATAAAGTGGAAATTTTAACACCATTTTAGAAATGCCATCC
TTCTAAACAGGAAGGCGGGGAAGAGCTGACCTGAGCGGCAGCCTATGTGATGTGAAAGCCCACCTGATGCCCACCTGTC
ACCGTGGCGCTCTTATCAGGCAGTGACGGGAGGCAGGCTGAGGAGAACTGAGATGTGAGGCACTCAGCAGGAGCTAGCC
AGTTATTCCCCTTCTGGTGGGGGCGGCTGAAGTATGGCTGGGTTTCGGACCACACTAGATGGTTATATCTGCATTAAGA
TGCCCCAGGTCCAACTCTATGGGTAAACACAAACCAGGATGATGTCACCTCCTGAATCCCTGATCTGCCTCCCAGAACA
GACTGGGGCACTCCTACATCCAATTGGAAAATAGAGGTGGACACTAGCCATAAACCAATATCAGCATCCTATTCTCTCT
CTCCCTTTCTACCCCCATGGCCTGACTCAAAGTCTGGGTGCCCTACTGAGACCCTGGTGACCAGCTCTACCAGACTTCC
TTACACCTAGCATGCCTTGTGGTACCTTACACCAAACACTTGGCCATGGGTCCTCCCCGATTAAGAATACCAGGGGCCA
GCAACACTACTTACAGAAAGTTAAAAAAAAAAAAAAAACCTTAAACTAATTATCAGGGGTGGGAAAGTGGATTGCATCA
TCCTTTCAGTAATTGTCTGGGTACAATTGAGTCAGCCAAGGTTTTTAATGCTGAATCTCCAGGGTCTTCAAAATAATCT
CAAGTTCCCTGTGAATCTTCAAGATGACCATAGGGGTAGTCACAGTTCCTAGGCAACTTTGCCCAAACAAACGTTTCTA
GGAGGTACAGCTCTTGGAATGAGTGTGTTGGTGTAAGAAAGGTTAAAGAGCCATGGATCCTCCAAGTGACTATTAGGAT
TGGGCTCACAGCCCTCCTCAGGGGTCCCCCACCCCACCCACCCCCCGTACTGACTTGGTTTCCCTGTGTTAACAGCCTA
CCCCTGGGACAGATCCAGTTTGAAATCCATGCCCCTGGACCTCCGGCTCTTTGAAAAACTGGATGCCTCTGCCTCACAG
GTAGGAGAATTGACTCCCTTGGCTAAAGGCCCCAAAGGTGACTTGGAGTTCCCCAAGTCAGGATAAAAGAGCCTACCTC
CAAGGCCTCTCTTGATCATACACTGAGTAAACCAGATGGATGGTCTATGGCTGTCCTTTCACAGTGTACCTTCGTTACT
TTGTAATGGCTGTGATCAAATAACTGGCAAGAGGCAAGATGATGGAGGGAGAGTTTGTTTTGGCTTACAGTTTGAGGAG
GCACTGTGTCTCGGGGCAGGGAGGGTATTGAGCGTGAGGTGACTAGACAAATTGCAACCCTAGGCAGGAAGAGAACAGA
CAGGAAATGGAGCAGGCTATAAAACCTCAAGGCCTGGCCTCCAGTGATCTACTTCTTCCAGCAAGGCTCTGCCTCCTGA
AGGCTCCACAACCTTCTCAAAGAATGCCACCAGCTGGGGACCAAGTGTTTAGATACATGAGCCTATGGAGGACCTTACA
CATTTCAATACCACCAGTGTGGATGCGGAACAGGAAGCCCAAGGGTTGTGTTAAACCCATAGGCAATGCCTTAGTTACT
TTTTTCTTGCTGTGACGAAACACCATACCTTGGGGAGAATAGGGTTTATTTCATCTTATAACTTATAGTCCCTTATCCA
AGGACATGGAGGCAGGAACTCAAAGCAAGAAGCCTAGAGATAGAAACTGATGCCTAGGCCATGAAGAACACTGCTTACT
GGCTTGCTCCTCGTGGCTTGCTTTCCTGTAGCACTCAGGACCACCAGCCCGGAGATGACAATACCTCCCACGTCATTCA
TCAGTTAAGACAATAAGACAAGCTTGTCCGCAGGCCAATCTGGTGCGGGGTATTTCTCAATCGAGATTTCTTCTTCCCA
AATGACTCTAGCTTGTGGCAAGTTGACATAAAACTAGCCAGCGTAGATCAGGTTTAGTAAATCTTAACCATGACCACAG
GCTGCTGTAGCTACCCGAATACCTGCCTGGCTGGTCCCAGATGGGAAGAGGAAAGCTGAGGTGGCTCTAGCACCTGGGC
TTCAACTGGCCAGCACACATGCCCTGTCCAACTCCCCTAACGCCACACTATTTATCCCCCACCACCCTCTGCCCTCCTG
GGCCCCGTTCCAGCTTCCTCACTGGAGAAAGAGTAGCTGGCCAGATTTGGTGGGTGTCACACTCCCATCCTCCCCTGAC
CCCCATAGAATCTGATTTTTCTGCTCCCAGGTGACAGTCAAGAGTGGCCTGAATGAGCTGGTGAGTGACTTGCTCCAGG
AAGCCCACAGCGATCTGGAAAGGGTCAGAGCCATCTGGATCTGGATCTGCCACCATATAGGTATGCCACCATATAGGTA
TGCCACCATATAGGTATGCCACTAAGGGGGTAGCACCGGGGACCCTGGATGCCCCTTCCTTCAGATCCCTGCTTCTGTT
GCCTCATCAGAGTGAGGAGTTAGGTCAGGGGGACGTGCCTGAAGTAACCAAGGAAGGTAAAAGCCACCCTTCTGAACTG
GGCCTTCACCCAGAAGCTGACCTCCCTGGGTCCAGGATTCCTAGTAAGCCCCGCTCTACAGAGATGAGAACCCAGGACG
GCCCATCACCAAGAGGGTGACCACAGACTGAGGAGGACACAGCTGTATGAACACCAGAAGGTCCTAAGACAAGTGCCAG
CTAGGGCAAGTGCTCATATTCCTGTCTCCTGCTGTCCCACCTGTGTCCCCGGCCGCTGGCTTCCACCTACTCCCAGGGT
TCTCCAAGCCATCTTAGAAACCTGGGCCTGAGCGGCTGCCCACTGCTCTCTGAGAGACAGTTAAGGGAATCTTGCCAGG
TCCTGACAAGTCACAGGCTTCCCTTGCTGAAGCCTGCAAGTGGGGGAGATGGTGGCCAACTGAGTGGCGGGGTGGACCC
TTGTTCCTTTGACATGAAGAACAGCCAGTGTCCCACCTGGATTCCCCGAGGCTCTTCTTTGTCCTCCAGGTGCAACTTA
GAAAATGTAGTCACCCAAGGTCTGAACCTGAGGGCGGGTGCCTCCTTCCCTACACCACCACCAGCTTTCTTCCCCCTCT
TAGACAAGGTTTCATTAGGCAGCCCTAGCTGGCCTGGAACTCACTATGTAGACTAGGCTGGTCTTGAATTCACAGAGAT
CTATCTGCCCACCTCTACTTTCTGAGTGCTGGGATTAAAGGTGTGCGCTATGAGGACCAGGTGGTCCTCTCCGCTTTGG
CCGCGATGTCCCTTTTGCCACCTACACAAAAGGTGACCACATCTGGCTAGGTTGGGGTCCTGTTTCTGTTCATGCCACC
ATTTCCTCCTGAGCACAGCTGGGGGGTGAAGGCTATCCTATGGGCTCTGTGGCTTTCCCTTCCTCACTGAGCCATCCTC
GTGCACCTGGGAGCCTTGCGTGCCCACAGCCGCCTCCTCTGTCCTCCAGAGTACGACGTTGAGGCTGCCCAGGAGAAGG
ACCGCCAGGCCTTCAAACCCACTGACATCCTGAGGACCCAGAAGACCAACTGTGATGGTTACGCCGGCCTCTTTGAAAG
GATGTGCAGGTATGAAAGACAGCCTGTGATAAGCAAGAGTCCCCGGGCTGTCACCCGACTGCCTGCTTCTTTCTTCTCT
TTCGTCCCTCTGTGCCTCTGCCCCCATTGCCAGGTCTGGAGCTTGAGTTTGCAAACTCTGCCGGGGAATCCAAGTGAGT
CCTGAAAAGGTCAATTTCTCCCTTTTTAACACCAGCGTAATTGACTTTGGACCATCTGCCGGGCTTCGCCATGCATTAA
CATAATGGCAGATGAATGCACAAAGATAATTTGGTTATAAAAGGCCCCTTTTCTGCCTTCTCCCTTCACTGTGCAGCTG
AAGGTTAAAAGAATGGCAGGCCTGGAAGCCAGAGGACAGTGGCCGCTGACGCCAAGGAGACTATGAAAGGCTGTCCTTT
GAGGAGGGAGAGGGGCCGTGCTTGGAAAGATCCATTGGTAGGAAAGCTCAGTAGCCAGGAAGTGGTTCTCTGTGCCTTG
GGGCCCCATTCTCAGAGCTATGGCACAGACACAGGCCCAAGTGGGAGAACTGGCTGAAGTAATTCTTGGTAACATCAAG
ACAGTCTCAAGTCCGGGGGAGCCATACGGATGGATTTCTGAGGTCACCTGGGTTCTCCTAGGGAACAAGCAGGAAGTGA
GTGATTGGCTATGTCCCTGAAAAGCTGTGAGTAGGTCTCAGATACCCAGTGTCCACCTATCGGGACTGCAGGTGGCAGT
TGTCAAACCACAGTAACAGATGCTCTCCAGTCGAGCAGCTGGCATGGCTTAGCGTTGTGCTGCTGGTCCCCTGTGGCCT
TGTGTGCACTTGTGTGTGCCCTGTCTTGAGATGCACACAACCCATTCTCGGCCTGAGCTCCAGTGCCTCCCAACCCTGT
ATTACCGCTTCCAGGTATAGGCAGATACCTGGTTCTGGGTCCCTCTAGCCCTCCTGCCCAACTCCCTGCTCCAAACTTC
CACTTCCCATCTCTGGGACCCTAGCCTCAGAGACAACCTGGGCAGGATCCATGCTGGCTGACCCACTGTTCTCCCTCCA
GCAGTACCTTCTACTTCTCCAAGTTCCCAGTGACATCCCCAGCCCCAGAGCCAGGTGCCCCATTGTCTTTGGGCATCCC
TGTCCCTGGGTTCCCAGTGTCTTTCAAAGTCCTCCAAAACTTGACCTCAGCCACCTGCTGGCTCTGCACCTCGAAGCAA
CACTCACACAACACTTCCGCTTCCGCTTCCCATGAGTGCACAGCCCTTCCTGGCATTTCTCTAGGCCCCACCCCAGAGC
CTCCTTCCTATCCTTTGACATCTGACCTTGTCTACTAACTTGTGTGAGTTACATTAGGTTTTCATCTTGTAGCTCTTTA
CTCCAGGTGATGATGAGGAATCAAATCCAGGGCCTTTCCCAGGGAAAACAAACAATCTACCACTAAGCCTTAGCCCTCC
CATGGTGCTCCAGACAGACTCTAGCTCTGTAGCTCAGGCTCGCTTTGAATTCACTGTGTGGCAAGGCTGGCCTAAGTGC
TGGGATTATGGGTATGCAACAACTGTTCTGCTAATAATTTTTTTAACTATATTATGTTTAGTTAGTTAGTTAGTTAGTT
ATGCCTCTGTCTCTCTGGGGGCGGGGGAGAGGGGAGGCATATACATGTCATAGCATTCATGTGAAGGTCAGAGGTCAAC
TTTCAAAAGTCAACTCTGTCTTTGTAACATGAGGGAATTGAACCAGGCTTGGTGGCAAGTGCTTTTACCCACTAAGCCA
TCTTGTCAGCCCACTAATAACTTTTTTGAATTAAAAAAAAAAAAATGCTTTCTGTAAATAGTCTCCAAAATGTACAAGA
CAAGGAAAACAACTAATTGGCTCTGAATCACCTAGGTAGAGTTGAGATAGTGACGCCCACGTGCCTTCCCAGGTGATAG
TGACACCCATGTCCCTTCCCAGGTGACAGTGACACCCATGTGCCTTCTCTGGCTTGTTCTCAGCACAGTTCTCTCTATG
TGCCTTTCTTCCTGTGTCTTAATCCCCCGCCCCTCTTCTGCCTCTCTGAGAACATCTGGCCCAGAATTCTCCACACAGT
AGACCCTCAATTGGCATTTACACACAGTAGGCTTTTAATAGGCATTTTCAGAATGGTGAGAAGCAAGAAGGATATGCCT
GGCAGACGTGTGTGTTTTCTTTTGAATAACAGAACACCACCGGGTTTTCAGTGCCTGCCTGGCATCTTTCAGAATGTGG
TTGTTTTCATAGGAATTTGTAAATTTGAAAACACTTGGATTTCTTTTTCTTTTTTTCTCATGATGGGTTTTGTTTTGAG
GTTTATTTTTTTAAGAAGTATTTATTTTATTTTATATAGAGGAGTATACTGTAGCTGCCTTCAGAAGAGGGTATCAGAT
CCTATTACAGATGGTTGTGAGCTACCATGTGGTTGCTGGGATTTGAACTCAGGACTTTTGGAAGACGAGTCAGTGCTCT
TAACCACTGAGGCATCTCTCCAGACCCTGGATTTTCTTTCTAGTTGATTCATTCTTTCACAATTTCATATGCATATTAT
ACTTTGAATGTCTCCCCCATAGTCCCCTCACCTCCTTCCCCTCCCACTAAACCCCTTCTTCCTAACAGACTCCCCCACT
CCTATTTTCATGACTTTGGGGGCCACTCTATTTTATTAGGGTTGTCTACAAGAGTAGGGGTTAGTTAAATAAGGGTAGC
TTACCACTGAGAAATTTGGCTCTCCTGCCCCCCCCCCCAACAACCCTTAACTGCCTAGAACTTCCCAGAAAGGGGTGGG
GCCTCATGAGCCCCGCCCCCATCCATCCTTGATGGACTCACCAGCTCTGCCTCCTCACTGCAGGACTCCTGCAGGTGGC
AACAGTCTTGAGTCTTGCAAGCATGTCTGTATGTCTCCTGAGTCTCCCGGCTCACTGTAGATGTGCCACAGGTACAGTC
AGAAAGACCAGGGCCCCCATCCCTAACTGACCACTTATGAGTTTTGTTCATTTAGGTAAGGCAGCTTCCTTTCAGGCCT
CGGTTTCTTCATCTGTATTTTAGGGACTGTATTGCTTTCAGGGCGGAACAGTAGAGCATGCCACGTGGAGGTGCCGCTC
AACTGACTTGGGAAATGCTATTTTTTTTTAAGACTTGCGCCATCTTTCATAAGCATTGGAGCATTTTTGCGGAACTTTC
CTACTGCCTCTGCTAGCCCACCCACATAGATCCAGAAAACTGACGCTTGCCTTGATTGACTCTGTCGGCTCTCTGACCT
GTTCCAGATGCTTAACAATGGCCACATCCTGCCCTGCAGCTCATCATTAAAATACACCCCCAACATTTCACATCAGTTA
AAGTTAGTCTCAACCTAGCGATGACCATGCACAAAATCCCTTGCACTGCCTGGGACACTTAACAGTAGCTCAGGGTTCG
GTGAACTCCCTTAAGTGTCGCCTTAAGGATCAGTAACCCATTCGTGCATGGCCTCCCTAAGCCCCTTGACCCCTGGAAT
GGCTGCCAATCATTTGGTAAATTCATTAGTTGGTTCCTTAATAATTGCAGACACGCAATCCACACCTGCTTATTGAGTC
TGTGGACTGCCAGGGTGGCCTCTTAGAAAGCGCCGTCCTCGGAAGTCCTGCCTAATGCTCTGCTTCCTGGCCAAGGGCC
TCATTAAAGACTGATTTAACAGAGCAGTTTGGTAAGGACTTAGATATTCCCAGGCATTCTCTGTTTCCCCCTTTGTCTT
TCCTACATTTCCGATGTGGTGTGTTTTGTTAGCTCTCTCGTCGCCCCGTGTAATTGCTGGTCCAGTTTCTTCCCGGCTT
TCTATCATTGAGCAGTAAGTCTGTCTTCAGCCCCGATTGCCCACAGCCTTAATTGATTCTGCCTAATCTCCTTGCAGGG
GCCAAGGTTAATTCCCCAGGCACCTGCCACTGAGTCAGCCTGCAGTAAATTCTCTCCCCCACTGTAATCATGGCCCCAG
TAACACCAGCCAGCAAATTTTTCCAGAGATTTGAGGTAACCGGCGCTTTATTGCCAGTCTGAAGCTTTGGCACTGAGCA
GGCTGGTTGTCACCAATCATATGTCAGCTGCATTTCCATCCATCTTCCATGACATCTTCTAGTCCCGTGAAGAGCAAGG
ACAGAGGCATGCCTGCTTGGTTAGCTGCTGGTGGCCAATGTGACCATAGTGGCCATCCAAGCCTCTAGGGAGATTCTCC
AGGAGCTGGGTGGTCTTTCTTATTTTCTTCTTTTTCAGCAGTATTGGAGACTGAACCCAGAGTCTTATATATGCTAGCC
AGCTAAGAACCCTATCACTGAGCTGCCTCTCCGTTAAGTTTTTGAGTCAGCATCTCTCTGAGTTACCCAGGCTGATCTT
CAACTCACTCGGCAACTCAGGCAGACCCTGTACTTGTGACTTTCCTACCTCAGCCTCCAGAGTGCTAGAACTAAAGTAC
TCCAGAGTAGAGAGAACTAAAGGCCTGCATCCGCCCAGAGCTGCCTTAAATAATTTAATTATTGCCAGCACTGACTTTG
TCACATAAGGCCTCTGAAAGGGACACCTAAACTCCCCCTCCACTGTGGCTACACTGGAGGAGGATACAAGCTCGAATTC
TTTCTCGGATCATCTCTTCATTTCATTGTATATCACCTACAGTCCTACATATGAAAGCAGGATGTTGGTCTGCAAAAGT
CCTGACATAGTTTGAAAGCTCTCAGACTGTGTCTCAGGACTGCTGGACCCCAGGATTGTCTTTACTTTAGCTGGCTGTG
TGTCACCAAGTCAGGTAGCCTTTCTCTCAAAGCCACAGTTAGACAGGAAACCTCTGAGATCATGCCTAGGAAGCCAGAG
GTGGACAGGTGTGTGGCAAGGATAGAGAGATACCTAAGTTTATAAGAAATCAAATCCAAGTGTTGAGAGAGATGCCGCT
GCTTATGCCCTGCTTAGAAATCACACGCCTTCAACAAGATAATGAAAGAAAAGAACGTTTCATTTAGCATGGTCAAAGG
CTGCTGTGCGCAGTGTTCCTGCCTGCCCAAGCCCCTCACCTACACTCCCAGCTTCTCACAGGCTATACCTGTATCACAT
AGCTCCTGTGGCAGGGCCTGGACCTTATCTGTATCAAACAACCATCAAGGTCTGGACTGTCAACTCCTCACTCTGTACC
CAGGGGACACACCACAGCAAGCTGGTATGCTCAGATCTTTGATAAGTGACAAGGAAATGTGCTCATAGCTCCAGAAGCC
AGCCAAGGTCACTAGGTAATGGCACCTTCCATTTGCCCACCCCTCTACTCAGTGCATATTCATCAGGCTGCAGAGGAAG
GTCAAGTGTTCTTCAAAGCCTCAGATCATTGGTTGGGTCCCCGAGCCCCAGCCCTGGCTGGGAATGGGCTGCTGCTTGC
TCCTGATGTTCTGGGTGTCGCAAAAGGATTGCTCGTGAGTCCTGCATGCAAGCTGGCTTTGCAATATCAGATGCACATC
CTCCCTGTGCTAGTGCCCCAACAGTGGTACTGTCAGGACTCTCAGATGGAGAAGCAGGATTCTGCAGCCTAAGGTTGTT
ACATAGACAGACGGCCTTGGGCTGTGTTTGGGGCTGGCTAGCCAAGCTACTCGGCAGGTAGAAACATGTCTCACCTGAG
ATCCCTTCTTCAGCACTTTTTTGCCTCAGTCTGGTGTTTCACAGATCCTCCTGCTTCGCAAGCACAAGCTCTTCTAAGG
CAGCCACTGAGCCTTGTGTTGGAACAATCTCTCACTGTGGTTCATTGTGTGTCATGTGCTGCCCCCCCCCCTTCACACA
CACACCCACGCCCAGTGCCGGTTCTTGTGCTTGACACACCATTTGTACGGTCAGAGCTCTTTCAATGATGTGAAGGGGT
CACGGATTGTGGTCTGATCAGGAACCAGAGAGGCTCGATAGATAGTTCTACAATCAGATAGATAGTTTTATGCCTGTGA
AATATTTAATTTATATAAAAACTACGGATTACTGACAACACTTTACATTGTGACTTCTGTCCAATTCATCTTACACATA
GTTGTCAGATAATAAAATAACCCCATGTCATACTCCAGCAATGCATAAACTTACAGCTGACTTCATTTTTCATCTTAGT
TCTGCCACAGGCAATCCCCACGGCCCTTTCTCCTTCTCCTCGGTCTGTCAGAGGAGAATTGCCCCCTGGAGATCACTTA
ATGCCAACCATCCTTTTTAGCCCATAATGAACCTGGCAAGGTCACTGCAGGTGACTGGTAGAGGTAGCAATAGAGTTCT
GATTTCCTGGCTGCGGTCAATGGCCACCCATTCCAGCTTGGAGCAATATAGGTACATGTCACTCATCCTTTCTCCCTCC
TATGGCTCGTTCCTGGGCTGGGCAAGACCCTTCCCCACTTGCTTCTCCTGGGTGGAAGAATACCTCCCACTCACTGTTT
AAAACAAGCTTCCCCCAGTTGGAGGAGACACAGTCTGCCTACAGGAGAGGCCAGTTCACTCCAGCACCCAGAGAAGATG
TGGATGCTAAATGCTCCCCACTCCTTCCTTGCTCCTGATGCTCTGAGCAGAAAGCAAGCACCAGACAGCAATCCAGACC
CCAGCGTTCCACTCACCGCTTGCCCTTCCTGCGAAGTGAGCCATTTGCAAACACAAAGGTTCCCAGGACTTCCCTTTGC
CCTCCACACCAGGCTAAAATTTACATACACAGTGTTTTCGAGCAACAATGGGGAATAATTCACTGAAGAATATAACCTA
AGTTTTCCCAATTCTATAACGAACACATAATCATATTTTAAAATGTTGTCGTGCTCCCTCCCCCGCCCCGCCCTGCTTC
TTTTTACAGTACTGGAGAGAGAGTCTTCGCTCTTGCATGCAAGGCAAGCACTCTGGGCCTGACCTACAGCCCCCCCCCC
AAAAAAAAGTAAGATGCAGAGGAATTTAAATAAACACACCTATAACCCCATTTCCGAGGGATCAAGAATCTCTCCCTAC
CCAACCCTGCCCTCGTTCTCCATGAATATATATATTTACACTCAGAGAGACTGTCTGCCGTGGCTTCCTGCCTCAGAAA
GACCTTCTGTTCTGGGACTCATTTTGGACCGTCACCAGTAGAAAACCCTACCACCAGGGTCTAGCAACTCTTGGTGTTT
GTCAACAGAATGTCCCCAAGTGTTCCTGAAATAACTTCACAGGGCCCAGCTGCTATAATATCGTGAGATTCCCTAGGGA
ACCCTGGAGACCTAACGCCTCTTTGGCACTGCGGACAGGAGGGGAGGGATTTAATTTAATTGATCATATAGTTCCCAAA
TAAAAGACACAAAACATTTATATTTAAAACAAGCTGTAACAGCACTAGAGCTGGGCAGATATCAGCCCTCTGTGTTATT
TTGTCTACTTCCTTGTCAATAACCCAGAGATATCACTTGCTTTGTTCCCCCTGGGCTGCTCCTACTCCAACTGGCTGGC
CCTCATGGCCATGGGGTCATGATCAGCCTACCTCATGGTATCTTCTTCCTCCTTCTCTCCTTGTGGTCTCTGCTTCAGA
CTCCAAGCCCAGAAACCAAAACCTGCCTACCTCTCTTCTGCCCAGCTGCAGGCCGTAGGCGTCTTTATTGACCAATCAG
GGATAACTTGGAGGGCAATGTTACATAGCGCCAATTGGGTGTACAATGAGGATTTCCTCGTCTCTGGAGGCAACCAGAC
GTTGGGGTTCAGTACTCAGCGTTACAATATGTAACAACAGACCAAACCTCAACAGGGACCATCAAATGTCACCCCCTGA
AAGGAGTCTCGGTGTGAGTCGCCTCAGCGTGGCTCTGCAGGGAAAGGAGGGCTACAGCGGATGTGGTGACATACCACCA
TCGTTATCTGCATGTGATGCAATAAGAGAAATGAGAATCTCTTCATTTTCATGCTACCACATTTGCCCCAGTGACCCAG
CAAATGACGCATAAGTGTACGTCAGTGACACTCAGTGTGGCCTTCTACTAAATGTGAGCTAAATACCCATCTCCATGGG
CAAGCCTCTCCCTACACTCACAGTCAGTATGTGATCTTCTGACCCCATTAGTCTAAGACCCTGTTGTTTAATGGGACTG
GAGATTCAGACCCAATGCATCCTGAGAGTGACCAGGCCCGGCTACCTGATGTGGGCCTCAGAGAGATGTTCAGGATCAG
CACTGTGTGGAGGGTGGGTAGTTGTTGAGGTGCAGATGGGGTTCCACTGACGAATCACTTTGGGAATCCAGGACTCAGT
AGACTTGTGAATTTATTATACCAGTTTTCACAAATGCAAGAAAAGTGTTGGGGCAGAGCAACTCAGCACCAGGGCCAGC
AGGCCCGGGGCCAGCAGCTCCATGTAGGTCAGACTGGTACTGTCCATTTGTCCATTGCCATGTTGGGGAAGAGCTGCTG
AAGAGAGAGGCCAGTGGGTGGTGGGGAAGGCAGGGCCCTCAGGGGACTTGCTGATTCAGCCTTTGGAGTGTTGGCCACA
GCCCAGACCTGGTTTTATGAGGTCTCTAAAAGTGACAGGCACTCCATCTCCCACAGTTCAACTTTCCCCAAAGTGATAC
CTGCATTGCTGCGTGCCATGGGGATACCCTGTCAAAAAGACAAGGCCAGCCAGTCAACGTTTCCACTCCTCGAGGAGCA
GGTAGAGACTTCAAAATGGTGGCTGTCTCAGTCAGGGTTACTATTGCTGTGATGAGACGCCATGACCAAAGCAACTTGG
GGAGGAAAGAGTTTATGCAGCTTATGCTTCCACATCACAGTTCATCATCGAAGGAAGTCAGGACAGGAATTCAAACAAG
ACAGGAACCTGGGAGGCAGAAGCTGGTGCAGAGACCATGGAGGAGCCCTGCTTAGTGGCTTGCTCCTAATGGCTTGCTC
AGTCTGCATTTTATAGAACCCAAGATCACTAGCCCAGGGGTGTCGCCACCCACAATGGCCTGGGTTCTCCCCTTATTAG
TCATTTAGTAAGAAAATGCCCTTTGATCTTGCCTACAGCCCGATCCTATGGAGGCATTTTCTCAATAGAGGCTCCATCC
TCTGATGACTCTAGCTTGTGTCAAGGTGATGCAAAACTGGCCAATGCAGAGGCCCAGGCAGGTAGTCAAGGAAGGATAT
TAATGCACCATGGTGCTTTCTGTTCCTTCCTCATGGAGGCCAAAGCTGATGGTCTGGTCTCCTGGCAGCCAGCCCATTC
TCCTAGGTTGGTTCTGGACCAGTTTGCCTGCCTTCCCAAGCCCATGCAGAGGGGAACAGCCAGGAATCAGAGCTCAGCT
ACCTCCTCTCTCATCACCAGCACCTGCCGGAGGTATGCTGGCTTCCCCGCCTCTGTGTGCAGCTCTTTCGCATTGGTTT
TCTTCCAAGAACATCTCTGTCTCCCAGCTCCCTGCATTCTCGAGGCCGTCCTCACTCTTGTCCTGATTTTTCCTCCTGA
GATGCTGTACATCGGATCATGGCAAGCAGGCCCTGGGGAGACACTTCAGAAGTAAGCTGAAGAGTCTCAGGGGAATAGC
AAACAACAGGGCTGCAGTCAGTCTGCACGGGCAGGGCCTGCCAGCACTCTGGATCCCTGTCCACACACCCCTAGCACTC
AAGCCCCTTCTCAGACCCATGTCCCATCCCCAGGGTTGCTGGTGTGCAGTGTGTCACCGTGCCTGGCTACTCCAAGGGC
TTTGGCTACCAGACCGGCCAAAGCTTCTCCGGAGAGTTCGACCACGCCTGGAACGCTGTGTACCTTGAGGGTCGTTGGC
ACCTGGTGGACAGCACCTGGGGCAGCGGCCTGGTGGACACCACCACCTCCAAATTCACCTTCCTGTAAGTACGAAGCTC
AGTTTTGTCCGTGTCTTTCCTATCTGTGTGTGGGGTGCAGACAAGGTTGAAGAAGGGAGGCATGTGGGGGAAGGGAAGG
GCCAAGTGTTAGAGGAAGACCGGACAGCCTTGCCTAGCTACTCTCGCCTTCCCAAAAGCTGGTTCCAGGTCGCTTGCAA
GGAGCTGAATTTTCTTGTCACCTCTGCCCTCTGTGACTCCTCTGACTCTCCATGAGTCCATAAAGCTAAACCCAGGAGC
CCCTCCTGTTTGCCCCAGGTCACATATGAGGAGGGGCGAGGCTAAGGTGCCTCCTTTGTGCTCCATCTTCTCCTCCAAT
TCCTGTTCTCTGAAGGAGGAAGAAGGATGCAGTTGGCAGAAAGGAGAGGTGTAGGTGGGGCTAAGCCACCTGGGTCCTT
ACGGTGCAGTCTGGATCATCCTCTAAGGCTTGTTCAACCCTGAACTTAACCTTCAGCCAAGCATGGCATTTTCATGGGT
CGTGATGTCCCTCAGCAGCCTAGATCCCCCTTTATTTGGGAAACGAACTGTTTCCTCCAATGTGTGCAAGTGAAACTTT
GGCCCTCCACACTCACCCTACATAGAGCTGTTACCCTCAACAGTGCCCCCCTTAGGCGGAGCAAAGCCCCCCAAGCCCA
GGCTGTCTCCCTTTCCTTCTCTTTGTCTCTGTCTCTGACCAGCCCTAAGGAGCCACAGAAGTTCCCCTGCTGCATACCC
CCTTCCCCCTGGCACAGGGGCAAGAAGGGTCCAGCGCAACTAGAACACTGTCCCCTTGCCTCCCTACCTCCCCATCCTC
TCGCTGTCCACACCCGCTCTACGGATCATGGTTTTGGTCCTTTCAAACCACTTTTGGGCATTAAACAGAGAGAAAGCTG
TCCTTTCTACCTCACCCTATCCATAACCTCCCAAGGCTCTGCGAGCAGGCCCTGAGTTCATGCTGTGGCCTTGTCTCAG
AAGGGACCTCAGATCCCCTGTGGCCCCAGGATCTGCACACAGCCACCCGAAGAGTCTCCAGTGCCCGCATTAGGACCAC
AGATCGTCCTGCCGACAAACTTCTGTTATCAGCAACACCCAATCCAGTGGTTGGGTCAGCTAGACAAAGATTTCCCAAA
GCTCCCCCGTGAGATACCCGGATGTGGGTGATAATGGTCAGGCCTACTCTCACACCCGGCTCCTAACCAGGTCTTCCTC
TACTCCAGCTACAATGAATTCTACTTCCTCACCCACCCCGCCCTGTTCATCGAGGATCACTTCCCAGACAACAAAAACT
GGCAGCTGCTGAAGCCGCCCCAGTCTCTGAGGCAGTTTGAGAACAGCATGTACCACAAGAGTGAATTCTACAACAAGGG
CATGCTGAGCGCCCACCCGGAGACGTCCATGATCAGAACAGGTAAGGAGAAGCACAAGGCGGAGCCCCAGTGACGGCTG
AAGCTCCCTTCCCACTAGAGTAAGCCAAGAAAATCAGACCACCAAGCTTAGCTATCTTCTATGGCCTGAGCTGACACTC
ATCGCCCCTTGACACAGCATGGAAACCATGGCTGCTGGGCGTTTGCAACACTCTTAAGGGGCAAGGCCTGACCCCAGGA
CCAGACCCCAGGCAGAGAGGATAAAGAAGTGTCATGGACCTCAAAGGACTCCCCCTAAAGGACTGTTTCTCATGCCTCT
CTGGGGATCCAGAGCCTGGTCCTGCTCTTGGCGCACCACAGCAGGGGTCAGGAAGGCTGACCAGCCCACCTCCCCCTTC
TCATCAAAGCCCAACCTGACAGCACCAGCTCTGCCACCAGGAGGCCTCCACACATCCCAGACAGAAGGCCTGAGATCCC
AGAAGCAATCAACTGGGGGGGGGGGGTGGGCATGAGGGATGATGGCCTTGACCCCACCCCACCCCACCCCACCCCACAG
GCTGATGGGATAAGGGGACAAGGCAGGGGGGTCCACGAGGGGGCAGTCAGGAGCAGGTACCTCTTGCCCATTGCACTTC
CCATGGATTCTTGATCTGGATGCAGAGCTGCAGTGGTCTCTGGTTAGTCACAGACACGTGGCCGCTTCAGCAGCCCACA
GCTGCTCACTTCAAAGATCATTTCCTGCACCTGAGTCTGGCAGAGGTCTGCGGTCTCTATCGTGAGACATGAAGCCACA
AGAAAGACTCGAGCAGACCACAGTGCCCTCGTGATCATACTGCTGAGTTGAGTTTGCCTTTTTTTGGGAGATGTAAAAA
TGTAAACACCAGATTTCTTAATTTATACTAACCTAAAACTCAATTCCATATTCATGCTCCTTATTGTAAGAATATTTCT
TGGTGCCTGTGAGTTGCCTTGAGAATAGAGCTTGAAAAGAAGCAAGGGAAGGGGGCAGGGCGTGAGGGTAGGGTGAAGA
GGAAGGAGAGAGTCACAGATACAGGGAGGAGAGAAGAGGCAGAGAGAATAAAGAAGTGCCAGCCACAGTCCTTCAGTGG
CCTTTGGGGTCCCCAATAATTCACCTCTTCTAAGATGCTCAGCACACTTCTGCCAAATGTTCTCTAAGACAAGTGCTAA
GCTCTTCCAAAGAGCCTACAGCCCAGACAAAGGCAGAACATAGAAAAAACACTCCCATGATTCATTCTGGAAAATGGTG
GGTTCTCTGGAAGAGGCCTCCAGCATCCCTCCAGCACCCCTCCAGCATCCATCCCTCCAGCATTCCTCCAGCACCCCTC
CAGCATCCATCCCTCCAGCATCCCTCCAGCATCCCTCCAGCATCCCTCCAGCATCCCTCCAGCATCCCTCCAGCATCCC
TCCAGCATCCCTCCAGCATCCCACCAGCATCCCTCCAGCATCCCACCAGCATCCCTCCAGCATGTCCAGCTCACCTGAA
GGGCTGAAGCAGACTCTTTGCAGCCAGGTCTTTAGGGTGAGTCCCCACCATACATGGAATGGAGGATTAATAGTGGACG
TGAACCAGACAGCCTTCTCCTTTGCCTTTCTCCATCCATGAGCGGGACTGGAGAAGTTACTGGGCAAGGGCTGGGGTGG
TCAGCTTCTAACAGAAGCCATGGCCGGGCTCTCAGAGCAGACACTGCCTGGGGAGCCAGACAAGCCCAGATAGAAAAAG
CCTTCCTGAGCCAGTGAAAAGCAATGTTCCAGTAGAGGAGTACAGAGGCAAGGCAAGGGGGAGCTGGCAGGAGAGGGAA
TGGATTCAGTGGGTTCGCTGAGACCCAGGTCCAGAAGCACAGCTCTGGCTCCCTGTGTGAGAAGCAAGTGTTGGGAGGG
GAGTCCAGAAAAATACCCTGTTAACACTCCTGGGCTTAGACTCCTGGAGGAAAGCCCGTTCTGGTTATGGTGTGGTGTT
CATATGACAGCTGTTGATACCATAATCTTTTCAGGGGGGTGATACAAAAGTAATACTTCAGAGTTGTGGGGGGTGGGGT
GGGAGGTGGTGGATGGATAGCTCAGTGTTTAAGAGAGCTTGGAGCTCATCCAGACGACCTGAGTTCTGGTACCCACATC
CTTAAGTCAGCTCACAATCGCCTGTAACTCCATATTCAGGGGGATCAGTACCCTCTTCTGAACCATTACACACACACAC
ACATACACACACACACATACACACACACACACACACACACACATACACACACACACACACACACGCACACACAGCATTC
ACACAAAGACATATGTGCACACACATAAAAATAAATCTCTTTTTAAGACGTAAGAATTGAATGAGACAGCACTCTTCCA
ATACTTTTAGGCCCCTCCCTGCTCTGGAATCAAATAAAATATCTAGGCAGGCTTCATCTAATGTAAAGCAAGTGGGAGA
GTAGAGGAAGGGAACACATATGGCTAGGGTAGGGTTTTCTTGCCCATCCTCCACCCCACCCCACCCTCCCCAGTTATCA
CAGTTATCAGCTGTCTCAAAAATATTCACCCAAATGAGTCAGAAGAATACCGCGTTCAGGGCTGTCACACTGTTAAAAA
TCAAGCTTGGTAGAAATCTTCACAACCACGGACATAGACACACACTGTAAGTCACACCTCCCCCCCAGTGCTTAAGCAT
CCCCCAGCACACACTGGATCCGGCTCACCTAACCACAACTTCCTCTCCGCAGTCAATGGGAAAGCCACAATCACCATCG
AGAGCCGGGCCCCAACACTGTTCATGTTCATGCTCAATGGCAAGCAGGAGCACGGGCTCCTGAGCCTCAGGAAGAACGG
GATGAAGCTGGAGGTGTACCCTCCAACCATGGGCACCCACAAGCTGCAGATCTTCGCCAAGGGCAACTCTGAAATCTAC
AGCTCGGTGCTAGAATACACACTCAAGTGCAACTATGTAGACTTCTCTGTCCAGCTGCCCAGTGAGCTCCACCAGCCCG
TGGGCCCCAGCTGGTTCTCGGAGCAGATGGGCATCACGAAGCCCTCTCACTCTGACCCTATCATTCACACCAGCGATGG
CCGCTGCGCCATCAGCTTTAGCGTGGAAGAGGGCGTCAGTGTCCTGGCATCCCTTCATGGCGATGACGGCCCCATCACT
GAGGAGACACAGCGGCGCTACATCTTCCAGTTGAACCGAGGGAAGAGGACCGAGCTCAAAGTCCAGCTGCCCCATGCCG
GCAAGTTTGCCCTTAAGATCTTTGTGAAGAAGAGGCAGGAACAAGGGAACTTCATCTTTGTCTTCAACTACCTCTTGTG
CTGCGCCAACACCAAGGTGAACTGGCCCATGTTCCCTGAGAGCTTTGGCAACTGGGGACAGGACAATGAACTGCTAGAG
CCTCTCTCCGGTGTGCTTCCCGCCAACCGCAACATAGCCTTCAAACTAAAGCTGCATGGTATCGCCAAAGCTCTGGTGA
AAGGACAGGACACGTGGCCCCTCACTCTTAACCCCGAGGGCTACTGGGAGGGCAGCTGCAACACAGCTGGCTGCCAGGA
AGTCTACGTCATGGTGCTAGAGAATGCTAACCACAACTTCTACAGCTACATCCTGAAATACAAAGTGAATGACCAGTGA
GGGGCTGCATTTCTTCACGGTCCCCCAGGGTTAAGGCCAGCCCTGCCCAGGGAGGGCCCAAGCTAAGAGCAGAGAGCCC
AGGACACCACTAATCTGCATGAAACCACTTCTAGGCCTCTGCTTCAGTCCCCTCTGCCTCAATCTCCCTGCTGTGTCAC
AGGCACTGAGGAATTTGTGCCGCCAAAGGCTCACTCAATGTGGCTGTGGAAGAAGAGACAGAGGCCAGGGCCCTCTAGA
AGAAAAAGGTGCTATGTAAATCCAAGGTATTGTGAAACGTACACCCCTGCCTGTGCCTGCTCTTGTGTTAGGACTTTGT
TGCTGTAGAGCTGAATGGGAAAGCGGAGGTGTTGCGGGTGCTGAAAGCTTTCCCCACGATGCCTGCCTGGTGCTTGAGG
CTGAATCTATGAGAAAGGTGTGCTCCCCCTGCAAGTAACATGGTGTTTCTCCCCACTACTTCTGGACCCCAGGGAATGT
CGGGCCTGCCCATCTTCCACATTCCTAGTGCTCCTATTAGCTACTTCATGCTCTTCAAGAGCTTGCAAAGGGGGTGGGT
GTAGAAAGCAAACCAGCTGACCCTCAGCCAACTGGAGGCTGCTGTCTCCTGATGCCTAGCATCTCTGAGATGTGCTAAG
GGCAGGAAAGCAGCCGTGTGCACATTCGGATGTCCTGGCTGGGGACCAGGGATGGAGTGGGTTTGCAGGTCCAGCAACC
CACATGGAAGGGTGAAGACCCCGATTCTATGTGCCTACCAGAGTTTAGCTGAGGGCCTGGAGATTGCCCCACCCCACCC
CCCACCAGCCATCCTCAGGAGCCTCAAATGCCTACATAGCCCACTGGTAGCCTGGCTGATGACCTGGGCCCTGGGCAAG
CAGATTAGCCCCAGCAGTTATCAGATACCAGGGATAGTGTCTGATAACAAGATGTTGCTCCTTCCTTACAAGCCAAACC
AAGCTGAGGGGACCACCACCTGCCCCCTAGAGAAAGTGTTGGTCTCTGTCTCTGAGGAAAGCCCTATCATAGCCATTTT
CCCTCCTGTCTTTCTCTGAGATGACAACCCCTGCCCTGAGTTCAACCTTCCTTCCATTGGGAACAGTTGAGGGCCTCTT
ACTTGCTCCGGCCTGTCCTAAAAAGCCTGAGCTGCCTAGCTGGCCCTCAGCAGCACCTGAGGCTGGTGTTTCCATGGCA
ACAGCTGAGGGGGGAAAAGCAGGCTGCTGCAAACAGGGAAGGCCTGAGGAGAGCCCCCCGACAAGAGGTTCCTGCAGTA
GCATCCCAGTGCACAGATGTGCTCCAGGAGGGTCCCTACCAAATTTCAGGGTTCTAGGACTCTGCTCACTGTGCCTGTC
TCAGGACCACCCCCCCCCCCAATCCCTCACTGCTCTCTAAGGAATGTCCTCAGGCTCAGCTGCCGCCAGAGCCAGCCTG
GGATACTCACAGCCTCTACAGTCAAGTCTTTCCTCAGTAAGAGCCCATCAGACTCCTTCGATAAGTGGCTGACCCCTCA
TGCTAGATCCTGGGACTATAGAGGTGAACACAAAGGACTCAGCCATAGGCACCCGCATCTCTGGGTGGGCAGTCTGCCA
AGTGGAACCTATCTGAAGACCTAGGAGCCCAGAACTTCAGCTCTTCATTCCCTTCTGCCCCTTTCCCACAGGTCCCCAC
TGCTACCCACCTGATACCACATAGCCCCCCTCCACCAGACTGGGTAGCCTCACTTGTGATTAGCATGGCTGCTAGGGGC
TGAGCTGCCTTGGCTCATGTCCAGGGTCCTCTGGCAGTCTCAGCCCATCAGCATCTTGTCACTCACGCCTGGAAAACGC
ACCAAAGTGGGGTGGGGTGGGGTGGGAGGTGGCGTTGTCTCCACTACAAGAAGACGGTGGAGTTTGCACTGCCTGGAAA
CAGCCTTTCCTCACCCCTTTTATTTTATGAGCAGTAAGAACTGTGCTATGTTTTAACGAAATAGTAAGTGCTTCGGCGG
TCCCTGCCACTCGCTAGACACAGCCGCGACAGCCACTGCTAGCCCTGCAGAAAATGATCCCCATGTTTTTTTCAAAACA
GCATAATTAATCTATTCTGGAGCTTGTGGAGGATGCTACCCCACAAAACAGGAGCTGTCCTCAGCACCAGAGACCCAGA
CCGAGCCTCTCCCAGCGGCCCGGGTCAACCACACGCTCCGCCCTCCTCATATAACCAGGTAGTCCCTTTGTCAAGGCAA
CTGTAGGTGCACTCGGAAAAACGCCTGCGTTTCTTTTTACTGAAGCAAAGATACACGAAACCGAATCCAATCTTCATGA
ATTTTTACACGCATGCTGCTCAGCCTGAAAATTTGGAGGCTGCGTTTTGAAGGCTTTTTATTCAAATCCCCCTCTTTGT
CTGCTGGTCCACGAGTCTCATTGGCCGATCAAATGGCACGAGCGCCCCCTGGTGGATCACGTGCGAAGTTGCCACCGCT
GCAAACCCCCAAGCTGGCTCTTTTTATGATGTTTGTTAAAAATCACTAGGTGCTTTCTCGTCCCTTGCGGACAAGTTAT
GCTGTCTACGGCATCCACATCGTGGATAGTTATCCTCGTGGTTGATATGCCAATGTCAGCGTTGTCTTGCAGACTGTCA
GAAGAGCCTGGGGAGACTTCCCTGGCCTGGCGTGGTGAGAAGATAGGATGGGATTAAGAATTGAGCATCTTGCCCAGAA
AGCTTTTCCTGAGACAGAAACCCTGGGCACAGGAATGCCTGTCTGGGGTCGCTGTGATCACACTCATTACTATGGCAAT
GACAGGCTGAAACTGATCAAACTCAGCGTTTTGCCAGGCAGGCCCCAGGATGGGCGCCAGGGTGCAGAAGGATGGGGCG
TGGTGAGGCAAAAGGGTGCAATTAAGCTCTTTCAGCTTGCTTCAAAAGTGCCACCAGCTCAGCTGGACTGTTCTCTGTC
TGATTGTGAATATTTTGAGTTCTCCATATGTTACATTGTCCCACGAGGACGGACTCCTCCCGGCCCACAGAGATTAAAG
AGACGTAAATAGATTAACTGAAGCATGAAGGATTTGAGCTAGATATAAAGAACCATTTCCTGTTGTGAAGCAGGAGGGA
CACGTTGCCAATAAACTCTCGGCGAAATTCTCAAAGGACTTGAAAAGGAGAAAAGTTCCCGCAGTGGTCCGGCATTTTC
TTTGCCGTGCTTACTGAAGCGGTTGGGTTCTCCCTCCGCTAACTCGTAACTCTTAAACAGTACTGGTTGGCCGGCAGTA
AACACCAGGTTAATGGTGTGACCATATAGCTCACTGTTGTGGGATTTCAAAGGCCGTTTATCTGAGGTCTGCCATTTCC
TTTCATCTGGAGTAAGTAAAGCCGCAGAGGTGACTATTTCTCCCCGTGTGTGCTGAGTAGTGTGGGCCACTGGCTGTTA
AAGATGCTGTCCTCACATTGTTGCTGGGGTTCTTTTTCAATAAAATTTATATTCATTTTTATCCAACAATGTAATTATC
GATCCATCCTTATCGCTGCTCTTACAAGGAGAAAAAAAAAAAAAAACAACCAAGGGGGGAGGGCAACACTTTCATGTTC
AAAGGATGCTTTTAAAAAAAAGAAAAGATCAATGTCCCCGCCATTTGAACGTGATGGGGAGATGCAAATGGGGTGTTGG
GCAAATAGCGATTCCACATTAAAGAAAACATAATTAGGTCAGCCCCGGCCCTCCGAGGCTCACGTGCCTTTGAAGAATC
CGAAGACCACGCTTCCTTCATTGAATCTTGTTTAACTTGAACATTTTAAAGTCAGCCCAAATTTCTTTTTAGAGTGGGA
GAAGAAAAACGTGCCAAAATTTTTCCTGTCAACCTACTTTTGGGGTGCTGAATATATGCTATACGTTTCTTGATATTCA
CGCACACACTCGAAACTTTCCAGGTTGCCTAAACTGATTACTCGTAAATTAGACATTTAGAGGACTGGAGGAAAAAAGA
AAAAAATGAAAATCTAGACACCTCTGAGAAGCTGCTGTAACCATGGAGACCATTCTCATAGCTGTAGTGTGAAGGGCAA
AAAGATTTGTGTTCCCAAATCTGAGCTTATCGCATCCCTCATTGCCAGACACAAAAACCCGAGATGTGTTACACTTCAT
CCTTGGGGTGGGGGTTCACAGAGGAATGCAATCCACGGGTAAGGGAAGTTTCTAATGACACACTATGGAGCAATCGGAA
CAAACTGTCTCTAGGTCTTAGGTGCAGTAGAAGGACAAACTCTTAAATTATTGGAACGGGCTGCAAGTTCCCAACCAAG
AGCTGGAATAGACTGACCTCCGATGTTTCTTCCAGTCCAGAGGCTCTGTGAAGCTGTGTCTTTGTGAACAGATGATGGC
AGGCACCAGCCTGGGCTTGTCAGTTAGGCAACATCTGAAGGGCCCTGCTGTGTTAAGAGCCTGCTCCCTAAGCCCCGGG
CATGCTACACCGCACGCCTTTTACTTTCCTGCGGGCGGGCGAGGGGATGCACAGGCTCAGCAGGTGGACAGACCACAGG
GAACCAGCAGCCCAGACAGAGGCTGGTCCACTGTGATTTGCAGACAGAACTGAACAAGACTAAGATGTCTCGAGGAAGT
CTCTTTTCTTCCACCTCATCCTCAGACCTCAAGTCAGAAAAATTATTCTTAAGGCAATCAAATTCCTGCGGAGGATGCC
TAGGGGTGTCCTGTGACCTCTACCATCCTCCATGGCAAGATCTGGTCCATATGGAGGCTTTGGGGCCTTGTCTCATGTG
CTTCTTCACCCACTGTGAGTGCTCAGGCCCACCACTCCCTCAGCGCCACTTCTTGGGCCTCTGTGTGCCAAGGTGGGTC
CTGCCTAGGTCATTACTGTCCACACCAACCTCAGATCTGAACATCCACCCCCACCTGAGCTGTGTTCAGACAAAATGCT
TTTCATGATCCTTCCCAAGGAAGTTCATGGTCTCTGGAGAAGGCACTGGTGGGCAAGGTAAGTCTTCAAACAAAAATTG
ATTTTAGGTCTACAATCCTACCTCCTTAAACACACCTGATCCAGCTTGATCTCTGAAGCTAGGCAGGGCCAGATCTGGT
TAATGCTTCAATGAGAAACAAAGGCTTATTTTGATAGATAATCAAGAGAGTGCCCAGTCACCTCCTTCTCCTCTAGAGA
CATCATGTCAAGTGCTTCATTCCCAAGGATAGTGTAATGATGGCTCTTCAAAGAGGGACACAGATGAGGGGCCATGGAG
AGACGTCTCCCCTGCAGTTGAGTGAGCTGTAAGCAGCAGCTTTAGTTCAAAGGCTGCCTTCCTGTGATCCTCCTGGACC
AGCAATGGCATGCACTGATCACGTAGAAGCACCAGTCAGAGCCAGCATCAAGCTTTGGGAATTCAGTGTCCTGGAGACC
ATGGCTGTGCAACATAAAAGGCCAGCGATCGGGACCAGGTAGGCAGCACCTCAGACTGAGATGGGACATGACAGGAAGC
CCCAAAGCACAGGTCCCCTGGTGAGTCCCCTGATGGTAGGGGCTATGGCGACAATCCTTAGGATAATCAATAGGAGACA
CCACAATCCACACTACCCTTCTTTTGAACATGTTGGTCCCTGTTGTAGCGACAGTCTTCCCTCCCTCTCTCCTGGAAAA
GCCTTGCCTGGCCCTCATCCATCCTCCGGGCTCACATCATATTTGTGACTCAGAACTCTGCACAATCGGGTGATGAGAA
GGGATTGGAACTTCACCACTGAACTGCAATTTTTTTCCATACAGTTTTATTCAAAAAATATTTTCATACAATATATTCC
AATTATGGCTTTCCCCTCCCAGATCCTTCCAAACTCTCCATCCATCCACCGCCATGCCCTTTCTTTCTCTCATTTTGGA
AAGCAAACAAGCCCATCGGTGGTGATGCACACCTTCAACCCTAGCACTTGGGAGGCAGAAGCGGGTGGATCTGAGTTCC
AGAATCTCTAGGAGTTCGAGGCCAGTCTGTTCCACAGAATGAGTTCTAGGACAGCCAGGGATACAGGGAGAAACCCTGT
CTCAAACAAACAAACAACAACAAAGCAAACAAAATGGCAAAGAAAAATAATAATTTAAAAAGAAAGAAACACACACATA
CACCAACAAAAATTCAAAATTGTGGAGCCGAAGAGATGGCTCATCAGTTAAGAGCACTGGCTGCTCTTCCAGAGGTCCT
GAGTTCAATTCCCAGCAACCACATGGTGGCTCACAACCATCTGTAATGGAATATGATACCCTCTTCTGCATGAAGACAA
AGCATGAATATATATTTTTAAAAACACAAAATTGGACACCATGATACATACATGAGGAGATCAGTAAAACTGAAAAAAA
CTGAAAAAAAAAAAAAAAAAAAGACCCAAAGCAAGATGAGACCAAAAGTCTACAAAAATAGCATTGAGTTGGTTTGTGT
TGGCCACCGGCTGCTGGGTCCAGAGCTTACATGTGTGGCTAATACACCCAATGACAGTCCACTGGAGAAATTCAGTTTT
TCCTTCGCGAGTGGGTATCAACTGCAGAGAGCTTCTTGGTTGGAGACAGGAGCTCCGGTCCACTTCCCCCTTCTCAGAA
CTGAGAATTCTTGGAACACACACTTTGGTTCTGCTTCTTGGCACACGCTGGGTGCTTGATAAATGCTCAGTAAGTGTTT
TCTTGGGAAAACTGAGAAGGGATGTGAACGGAGCTGCCAGCAACCATTCCTTGAACATTTGTAGAGTGTTTCGTTTTCC
CAGAACTGGCCCTTGTGGTTTCATTCAACTCTGCCTGCCACAGCCTGGATGGTGAGAGAGGCCGAGAGTCTCAGGGGAG
GATCCCAACCCAGCGAAGGGGGAATGGCTGCCCTGGGGATGGGGGGGGGGGGGGGGCTGAGGGCTGGTTGCTAGTGACC
TTCAGAGACAAAAGTGACCCCTTTACCTAGACCTCAACTTATTCTTGAGGCTCTCTCTGAGGGGGTGTAGGCTTAGTCT
GTGGACTGTGACTGAATGCAAGATTTCTATGTGAAGGTGGGCTCAGGGCCTCCTCATCTATGAAAGACCCATGGAGTTG
CTGTCCCCATATCTAACTGTGGCTTGGTCTGTTTAAAGGAGGTCTGGAACATGAGCCTGATGCTTCTGTTTGACCCAAA
GTAACACAAACACGCTAAAGCTCCCATTAATTGTATCCTAACCTGCCCAGAGCTCAACCAACAAACCTAGCTGCAGGAG
ACTCTCTGAGGCCCCAGGAGAACCTATTATCATATGGAAAAGCTGCAAAATGGCAAGTTTTGCTAATTGGGACTAAAAG
GCACAATATTTTCTCCTGGCAAATCAGGAGTGTGCTGGGCTGTCACAGAACTCATTATCCCTCTCCCTGTCCCTGCTCC
CATCCTTATCTTCTCTCTGCATGTGTATGTGGTATGCATTTCTGTATGCATGCCCACATGTATGGAAACACATGTGTGT
GCATGCATGTGGAGGCCCCCAAGTGGACATCAGATGTCTTCCTCAGTCACTCTCCACTGTATGGAAGCAGGGTCTCTCA
ATTGAACCCAGAGCTCATTGATCAGCTATTGTAGAAAGCCAGTTTACCCTGGGGCCCCCTTATTCCAGTCCCCCAACTT
CTGAGGTTACAGGCACTTACTCAGATTTTGCAAGGGTCCTGGGCAGTGAACTCTGATCTTCAAGCTTGCACTTTATCCA
CAGGGCCATCTCTCCAGCCCCGTTTATGGATATTTTAAACAAGGTTGCTCTTGGTATTGAGTGGAAAAGAAACAGAATC
GGCTTGGTTGGTTTGGTGACTGAGGGTTACTTGGACAGGTCCACGGAGACATTCCTGCCTCACTGGGGCTGAAGAGATG
ACTCAGTAAAGTGCTTGCTCTGCAAGCAGAAGGAGCAGAGTTTGGTCCTCAGCATCCATGGAAAAGGCCAGGTGAAAGG
GCAAGGATTTTGTCACCCCAGCACTAAGGATGGGCGGATCCTTGGTGCTTATCGGCCAGCAAGCCTAGCCCAGCTGGCC
AGCCTCAAGTCCCAGAGAGAGACTTTGTCTTAACACAAAGGTAGATGACACCTGAGGTTGACTTCCTGGTACACACATG
TACACACACTCACATGGGTAGGGCACATATGCAAAACACACACATACAAACCATTTCCTCTCTCAAGCCAACTGTATAG
ACATTTGAACATCACCAACTGAAGCCCTAGGTTTACGAGGAGTCTGTGGTCCCCTCTGAGCTCAGTAGGAAAGACCCTC
ACAGTGACCAAAAATCATAGACCCTGGGTGGGAAGGGAGTGTTATACATATTCGCCAAGCCTGACTAATAGCCAAACAG
GAGCCAAGACCAAAGGAGGAAGGAAATTACTCAGCTTCATGGGGAGGAGGGAGCTCTGGCTCCCTAAAGTCAGCCCCAT
GGCTGAGAACCATCCAGACACAAAACAATAGGTCCCTGGATGCTCTGGAGGGCCCTTTATGCCCCTGGGCTCTGGGCAG
GATCCACATGGCCTCCCAAGTTAAGTTTCTCAGTCCAGAGAAAAATTGGAAAGTTGGAGCTAAACTCACCAGTGGAGTG
CACCTCTGATTAAATACTGTCTAGCATGCAGAGTGACCAGTGATTGGTCATTTCAAAATGAAATACTAGTCCCGCTGAT
CTCAGTTACTCCGCGGTGTTGAGGCAAATGTTATGTCCTCCTCGCCTCTGATCTTATGATCCTGGGAGTGTTAGGAGTG
CCTGGGAGTGGAGCCTCCTCTGCAGAGTTCACAACCAAGGTCTTCTCAGGGCACCTGCCCAGACTGAAGGGCTCAGGAT
CTGCTCTTAAGGGAGTGTTGTGAACAGGGCTGAGAACCCGCTGAGCGAGGCTTTAAAGATGGTGTCTTTCCTCCAGTCA
ACATTGCTACCTCAGAGTCTCCCAAAAAGAGGCTGAAGAAATATAAGTACCAAGGCAAAGAAATGTCTTTGCAGTGACG
ACAGAGGATTGTTTCCAGCCTACAATTCTGGAAGACCAGGAAAGATGAGCAGGTGCTGAAGAGAAGAAATATCAATCTA
TTCCCCAGGGACGTGGTTTCTCAAGCACTAGTCAAAGAGGCCGCTTCACTTTGGATGACGTCATCCAAGGGGTGAATAG
TTCAGATATGATCCTGTGTTTCCTGGCCACCCAGGCTGCCAGGGAAATGCAATCTCAGGACAGGAAAATAACCCTCCTT
TAAAGTTGATTACTGAGGCAGGGCTCATTCCCAAGCTGGTGGACTTCCTGAAGGTGACACCTCTCCCTAACTTGCAGTT
TGAGGCCGCGTGGACGCTCGCCAACATTGCCTCAGGAACTTCAGAGCAGACCCGAGCTGTTGTGAAAGGGGTTGCCATT
CAGCCCCTGCTTGAACTCTTCCGTTCCCCAGACCTGACCGTGTCTGAGCAGGCAGTGTGGGTCCCTGGGAATATAGCTG
GTGACTGTGCAGAATTCAGAGACAATGTCATTTCCAATAATGCCATCCCACACCTTATCAACCTTATTTCAACAAACAT
ACCAATCACATTTCTTCGGAACATCACATTGACTTTGTCCAACGTATGCCGAAACAAAAATCCACACCCTGTGAGGATG
CCTCCATGAGGCAGATGCTACCTGCTCTCTGTCAGCTCCCACTGGATCATGGTAATGAGATTCTCTCAGATACCTGCTG
GGCCCTGGCCTACCTGACTGAGGGCAGCAAAGAGTATATCCATCATGTGGTGACCACCGGCATCCTGCCCGGGCTTGTA
GAACTTAAGACCAGCTCAGAACTCAGTGTCTTGACCCCTTGTCTCCACACTATGGGCTGCATTGTGGCTGGAACAGATG
AACAGACCCAAATGGCCATCGATGCTGGCATGCTAAAGGTGTTGGGCCAGGTCCTGAAGCACCCAAAGTTCTCCATCCA
GAAGCTGGCGGCCAGGACCGTGAGCAACACAGCCGCTGGGCCCAGGCACCACATCTAACAGCTGATTTTGTAACTTGCT
GCCTGTCTTGGTAGACCTACTGAGGAATGTGGAATTAAAAATCCAAAAAGAGGTTGTGTGCACCATGGCTAACATGGCA
ACAGGGGCCTCTCAGTGCCAGCTGACTCTGCTTGCCCACTCGGGAATCTTGGAACCAATGCTGAGTCTGCTCACTGCCC
CAGACATGGAGGTTGTCATTGTTGGCCTTGACATCATTTCTTATCTCCTCCAGATAGACCACCTACAGGAGAAGAAAAG
GCTGTGTTTTCAGACAGAGGAAGTTGGTGGCTTTTAAAAAGTTGAGTCCCTACAGCACCACCACAACACTTAACATCAG
CAATGGTGCTCTGGACATCATTGAGAAATATTTATGTGAGGATGAAGACAACGACAGTCTGCCCAGGCCTGGACTGAGA
GTGTGAATACGTGGAGAGCTAAGACATCTCACACACCAGATTTCAGCAGAGTACCGATCAGTCATGGACTTTCACGCGG
CTTCTGGCGACAGCACAGACCACAGACATCCACATGGTCTCTGGTGGCAGCACAGATCTTGGACATCAGCTGGGCTCTC
TGCCACAGCAAGAACCATGGACAACACCATAGCCCTCAACAGCAGCACAGGTCAGTGACGTCAACATGGTTTCAGGGGG
CAGCACAGACCAGACACAATGGCATGACCTTCAGTGGTAAAACAGGCCACCGACATTAAGACCTCCCCCAGCTGCAGCA
GGACCATGGGCCCAAGCAGTGGTCAAAAAATATACACGGCAAACGTCTTTTACCAGCTGAGGAGTCTAACTGACTAGGG
ACAGTGTTTTTTGCTTTTGGTTTTGTTGCTGTTGTTAATCTTCTTTGTTAGTCTGTTTCTTTTGATTGGGGGATTGAGA
TTATTAATACACAGTTACTGCAAAGCAAAAGCACAAACAAACAAACAAAACCCAAATGAAATATTAGGTTACTGCATTG
TTTAAAGGGCTGTAGACTGAAGGTTAATGAGTTACAGCATCCTAAATAGTAATTTCCTTCTTGCTTCCAAGTGAGTCAA
GGAGAAGATGCACAGCCTAGACCTCTGCCACTCCACTCCCGTGGGAATGTCTCTCTACAGGAAGAGAGGGCTTGCCACC
TGGCTCATAGTACTTACCTCCAAGAAACAAATCGCAGCTTGGGCAACTGATCGATGGAACCCAGGGAGCCCTTCTTCTG
GGGGAATTCACTGGCCTTGAGCTTTGCTGACATTGCCAGATGACTGCCTCCATGTCACAAGGAATCAGCCATCATGTTG
CCCTGGCTATGCAGAGGACCTGGGCACTTACACCCCCTGCAGGGTGAGCAGAATTTCAGAGAGGCAGCCATGAGTGTCC
AAAAACAGCTATGCTGTGTGATCAGTGACAGCATACAGGAGGAGAGAGGTGCCTCCATAGGACAGTGGTGAAGAAAAGT
GAGGAGGGTTTTATTTAAAATACGAGCAGAGAGTGACAGCGTATACTGAAAGCCCAGCACCACAGAGGTAGAAGCAGGA
AGAGTCAGAGGCTAACCTGGACTACATGGAAAGATAGGTAGATCCTGTCTCTGAAGAGGAGGAAGAGAGATGAAGAGGA
AAAAAAAGTGGCCTTCGGATAGCAAATTGTCTAGTGAACATTTTGCCAATAAATCTTTATGTATGTGAACAGCAAAGAA
ACCTGAAAACTTGTGTTGACCGTTTTTTTTTATTACATTATACAAAATGAACTGAAGGGCCAGCGATCTGGCTCAGTGG
GTAAAAGTGCTCGCTGCACCAGCCTGATGGAAACCTGATGACCTGAGTCTATTCCCTAGGAGCCACACGGTAGAAGGAA
AGAACCAACTCCACATCCGTGCCACAGCATGCACGCCTGAGCTCACACACATCACAAGCACACACAAAAGGGACAGCAG
TGGGTACATAAAAAGTTTAAATGGAATGGAGCAGTTCCCCTGTAGGCACTTGGAGGCCGCTCCCACTCCTCCATGGCCT
CGGCTGCCCTACCCTGGTGCGGGCATGTGTGGGTCCCACACACCCTTTGCGCACAGCACAACGTCTTGAGAATCAGATC
GCGTTCCACACCTCCCCCAAGGAACACTGGGAGCAAATCACTGTTAACCTGCTGCGGGAGGGAATCTCCTCGGACTGTA
AAAGCACACCACTTGATGTTTTTTAAAGCTCAGCAAGAATGTGAATGTAAAAGTCCATTTTGAAAAGACGCCAATGATC
GGTCTTTCCAGGAAGATGGCCTCTTAGCTACCGGTCTCGCCTCACTGAAAGCCTCCTTGTGCCATCAGGAGCCATTAAC
AGATGATTGAAGCCTCAGGTTTCCCTCTATAAACATTGATTTAACTCCGTGAAGAGTTTTAGGCTCCTCCGTACCCAGC
ACGGGCTGGCCTGGGCTGTGGATCTGATTCTGTTTGCCAACGGGCAGGGATGCACAAAGGTGAAAGGTATGTGCCTCAA
AGTGGATTATTCTGCCCCTGATACCAGCTCAGCCAACTCGCACCTAGCCTGGCAAGAGCCACATCCTCCCTGCCTGTCT
CCTCGTCAGTGAGGTGAGAAAAATCATTTGCACCCTCTCAGGAGGATGGCATGAGTCAAGAACTCAAAAAAAAAAAAAA
AAAAAAAAAACCCGACCAGCACAACCCTAGCATAGTGCGCATGTGCTCTCTAGCAGTGTCCTAGTTGGGGGTTTCTACT
GCTGTGATGATACGCCATGACCAGAGGCAAGTGGGGAGGAAAGGGTCTATGTGGCTTATGCTTCCACATCACAGTTCAT
CATCAAAGGAAGGCAGGACAGGCTATCAAGCAGAGCGGGGCACTGGAGGCAGGAGCTGGTCCAGAGGCCATGGAGGGAT
GCTGCTTACTGCCTTTCCCTACATGGTTTGCTCAGCCTGCTTTCTTATAACACCCAGGACCACCATCCAGGGATGGCCC
TACCTACAAAGGGCTGCCCCCCACCCCCTATAAATCACTGCTTAAGAAAATACCCTTCCAGGCTTGCCTGTAGCCTGAT
CTTACGGAGGCATTTTCTGTATTGAAGTTACCTCCTCTCAGATGACTAGAGCTTGTGTCAAGTTGACATAAAAATATGC
AGCAGAGTTGACCCCTTGTCATCTTGACACACAAACACGTCACTGTTAAAGCCGCAACCTTTCCTTTCTCATCCATCCC
CACAGATCTTACATTTAAAACATGAATAGCTTGAAAATTCCCACAGTCTTTACAAATTCAAAACACATTTAAAGGTCAG
TCCATTTAAAACATCCGGTCTCGAGTCTTCTCTTTGTCGCCTTCGGAACAAGATGTAGAGCTCTCAGCTCCTCCAGCAC
CACGCCTGCCTGAATGCTGCCTGTGCTTCCCCACCATGATGATAATGGACTGAATCTCAGAACCTTGCCTGGCTGCCTG
GCAGGACCTGCCTGGAGCACTGTCCTGTAATTCTATTCAAGAGACCCTGGAGTTGGACTCCATCCTCAGCCACCACTGG
TGCATTCAGGGCTGCAGTGCTGTCACAGGAGATGACTGGCTACCTGGCATCAACTGCCCCCCCCCAATGATATTTCCAG
TCGTGTCTTTACTGCCAACTGAGCTCCTTCAATGTCCCCAGCTCACTGTCCAGACCCTGTCCTCATCAGACACCAGCCG
GAGGGATGAACACCAGCTGGCCAGACTAACACTCCCAACCCCACCTCGACCTGCTGCTGCTACTGCTGCTTACTGCTGC
TCTCCCTGCCCCCGGATGAGGGCTGTCACCCTGACTCCAAAAGTGGCCTGCAGCTGCCATGCCAAAAGGAGAGGCTGGG
CTGGGAGGGACTACCTGCTGCTACCAAGGACCTGGGTGTCACCTCTGTCCAACACTGAGAATAAAGCACTCCTTGGTCA
GTTAAAAAAAAAAAATCCAAAATCTCTTTCAAACACTTGAAATCTCTCAACTACAGGCTCCTGTAAAAAACAAAATTAA
ATTAAATACTTTCTTAATTCAAGAGGGAAGACCGAGGACACACACACACAATCTTAACAAAGCAAAACCACACTCCAAC
AGCATAAATAACTCAATGTTCAATATCTGGGATTCACTCACCATCTTCTGGGCTCCTCCAAAGGGCTTGGGTCACTTCT
CCGGCCCTGTCTTCTGCAGCACACACAAATTGTCATCTAGGCTCAGGCTGGCTCCTTGCCACGCCTGCTGCTGCCTTTG
GTGGTCATCCCATGGTGCTGACATCTCCAAAATGCTTGGGTCTCTTGTTGCAACTGGACTTCGCTTTCACCAGTAGCCT
CTCCACGGCTCTCTTCAGGATCTTCAACCTAGCCACACAGTGCCAAACCTCAGCTCTTGTAAATGACCCCTTCCCCTTC
ATGTGTTGAAAACCAGTACCACGTGGGTGGCTCTTACATTACCAAATTCAGCTTCCAGTACAAGGTGCAGCCTAAGACA
CCTCTGGAACACAGCTTCTATGTGCTGGCCCTGAGGAAGCACTTGCCGGAAGATTTCACCTCGGTGCCGCTGCTTGCTT
CTTAATCGCAGCTGATTGTTCGGCCCCAGCTGACCAACATCCACCATCCCAGCAAAGCAAAGGTTTCACTTTAGTGGTT
CTGGTATCTTGTTAATCACAGTTCATTTCCTCAGCCCACGCTGACCAGACCCACAGATTCTTTACTTAAATGGCCCACT
CGAGTCTTTGCTTCCCTCTGAAACTTTACAAGCCAGCCCTCCATCATCTGCACTGCTCTCAACATCCTTATCTTCCATG
CCCCTTCAGAACATCCCCCTGAGCTCTCAGCACTCGATGGCTTTTCTAACTGAAAGCCTCAAAGTCTTTTCACAATCTT
CCCAAAAACATGGTCATATCCGTCACAGCAATACCCCACCATGCTGGTACCAACTTGTCTTAGGATTTCTACTGCTCTG
ATGCAACATGACGGCCAAGAGCAAGTTGGAGAACAAAGGGCTTATGCAGCTTATGCGTCCACATCACAACTCATGATCC
CAGGAAGTCAGTACAGGAACTCAAGTTAGGCAGGAACCTGGAGGCAGAAGCTAATCCAGATGCCATGGAAGAGTGCTGC
TTATTGGCTTGCTCCTCTTGGCTAGCCCAACCTGCTTTCTTATAAAGCCCAGGACCACCAACCCACAGATGGTTCCATC
CGCAATGGGCTGGGCCCTCCACCACCAATCACTGATTAAGAAAATGTCCTATAAGCTTGCCCACAGCCTGATCTGATGG
AGGCATCTTTTTTTTAATTGAGGCTACCTCCTCTCAGATGTCTTTAGCTTGTGTCAAGTTGATATAAAACTATCCAGCA
CACATAGTAAGTAGTCAGTACATATCAGACAGTTCTGTGATGACATCTTAGTTGTCATGTGAGAGCGAACAAGACAGAT
TATCAGATCCTATTCCCATGACCTGCAAAATTAAGAGAAGACCAATTCCTATAAGTTGCCTTCTGAATGTCCTCTGTGT
CATGCACATGTTCTCACACAGAGACACATGAATACACACATGAATCCATGCAATACATGAATCCTTGCAATAAATCCTT
TAAACATAAGTATTCTCTTCCAACAAAAATGTGGCAGAAAGCTGGTGAGGGACACTTGAGATGGTGACATAAAAAGGCA
TGACAGATTCCTCCTCATTTTCTCTCTTATATTTTTCCCTAGGTAAGTGGGCACCTCGCTGGAAAGCCTTGTGAGGCTC
CCCCTCAGATACCGCTGCTCTAGAGCCTCTCAGCGGCACCTCATGAAGGATGTTGAGCCAGACTTTAATTGTTGATCAA
TTCCTGGTCCATGAAAATCACAGGGTGGTGGTTGGAAAGATGGGCTGGTGGTTAAAAGCACTGGCTATTCTTTCGGAGG
ACCTGAGCTCAGCATCCACATGGCAGCTCACATCCAGCAGTAACTCTTAGGTACAGAGGATCTGATGCCCTCTTCTGGC
TCCCGTGGGTACTGCATACACAAGGTGCACATAAACACAGGCAAAACACTTGTGGGCATAAAATGAAAAATAAAACTCA
TATGACACCAAAAGTTTATTGTTTGGGGAGGGTTTGTGACATAGCCACAGATAACCAATACAGTACTTTCACAAATGAA
AGACGCTCCCAAGATCCCCAGACCTGTCTGACTTATAGGAGCTTAAGTTCTCTATTATTGCTGGGCACAGTGGTACATA
CCTATAATCCTAGCAGGCACGAGCAAGAAGGTAAGTGTAAAGCCAGCCTGGGCTACATGATGAGTCAGTGTCTCAAAAG
AACAGTGACAACTATCCCTGTAGCACACAGAGAGCAGACGTGAACACAGACCGTTTCAAACTCGCTAGGATGAGCACCA
GAACACTGAAGACAAAACAGGCCCCCTGAAAAGCAATAGGACCAGCTGTCTAGGAAGCATCAGGAATCAGAATGGCTTC
ATGCTTCACAGCTCAGCAGAAGCGAGTACAGTGACTCCTGCCTTCAATAGTGCTGGAAAATGATATCCAACCTGGAGGT
CCTCCAGCTAAAATCCAATCAAAGATGAGAACGGAAAAAAAAAAACATTTAGAGTCCTTCAAGGTCTCTAAGAAAACTG
AACTCACTTGAGCCAGTTCTATGATGGAGTGCTACACCCAGCAAAGGACATGGACCAAGAGAGAGACCGAATACCTGAA
AAGAGGGACTCCAGGAGTTCCTGGGACCCTGGCAATGAGAAATCTGAAGAACAGAGGTGTTGGGGGGCAGGAAGATAGT
CAGAGAACTGTGTCACCATCACACTGAAAGGTCCTCAACGAGGCTTCCTCATACAGTGGACAATACTGAGAGGGTATGG
AGCACTGTTGAATCCAGAGGACTGCTTTGTGCTAACTCAGCAAGGAAAATATGTTCCAGAAACGGAACATCAATGTTTA
CGTTGTAATAATCCAAATGCTGTCTGCTGATGTGAGAAGATGTTTGGACATCTGGTAATGCTCTCAGCCTATCCCTGCC
TCCCCCATCCCACCCCACCCCTGCCCTATCACATCTCTGCTTCCCTTGGAAACACTTTGTAGGTGACAGAAGAGTTCGG
TGTGTGGCGTATTTGCTGTTTCTGGGGGTGTATTGCTATAGTTTAGATGCTCTGGTAAATGGTCTTACCTTTATGTTCA
TTTTCTCATTGTTTGTTTCTAGTCTGTAAAATGCATTTATTGTTATGCATGGATTTTATATCCAGTGAGGCATTCCTAA
ATTGCCTTATCAACTGTTCACAGTTCAATAAGGAGGCCCTGTTGGGCTCTTTGTGCACAGCATCACATTGCCTGTGGAT
AACTTTTTTTTTCCTCCTCTCTTGTTCTATCCTTTGTTTCTTCCCCTGTCTGATTGTTCTGGGTAGATGTGATGAAGAC
AGTAGGTGTCTGCTCCGTGTTTTCTACCTCAGCATGTGTTCAATACTTGTCCTGGTTAGGGTGAGGCAATGCAAAACTT
GTATGTCTAGAATAAACACAGCCTGATCTCACTATATTCCCCTTCCTGTATATCACGTGGTTTGGTTTGGCTTGGCTTG
GCTTGGCTTGGTTTGGTTTGGTTTGGTTTGGTTTGGTTTGGTTTGGTTTGGTTTGCTTTTGAGACAAGTCTCACTCTGT
AGTTTTGGCTGGGTTGAAAATCACTATGTAGACCAGGCTGACCTTGAGCTTGCAGCAATCCTCCTGCCTCAGCCTCTCA
AGTACTAGGAGCACAGCCATGGGCCACCATGCCTGTCTTATGTCAAAGGATCTGATTTGTTGATGATTCTTATTGATTT
TTAGTCTATACTCACAAGAAAGATTGGCTTGTCCTTTTCCATTTGCCATGCCCTTGTTAGTCTCTATGGAGGTTAAGGT
AACCTTGAAAGAAACGAAAGAACTACTGGAGAGCTGGTGCAGGCTTTTTTTTTCCCTCTCCTTAAATGTTCAAAAGCAT
TTGTTGGTGAAGCCACTGGAACCTGTGGTTTTCTTGGTGGGAAGATTTTCAATTAAAAATCAATGACTTTGAATTTGAG
TTCTGTTTCTCCGTGTCTCTGTTTTGAGATGCTGTGGTTTTTGAAGACTTGTCCCTTGGATTTAAATTGCTCTATTCAT
TGGCATTGAGTTCTTTATACCTATTTGTTAGTGTCCACAGGATCGGTCCCAATCTGATGTTTTTGTTTTTCTTTCAAGC
TATTCCTGATTTGTGCCTTCTCCCCTCCCCACCTGCTCTTCAGTGGTCAATAGCCTCACAACTAAACACAGTACAGAGT
ATCGCTACCCAACACTGGCTTACCTTCCTTCTGGGATATGCCCAAGAGAGCTCGAAATGGACCATAGGGCAGTTTCATT
CTAGTGTTTTAGGATTTCCTTTGCTTTTATTTTGACTTTTGGCCAGGGTTTTTCTTTTGGTTTTGTATGTGTTATGCGT
ATGCAGATGTGAGAAGTGATGGGTGGGTACCACACAGCACATGTTAAGATCGGAGGACAACCTCAGGTGTCAGTCCTGG
TGTTCCACCTTTGAGATAGGGTCTTTTTGGTGCTTGTTGCTCCATGGGTTAGGCTGGCCATGAGTGTCTGAGAAGTCAC
GGTCTCTACCCCTCTTTCCCCCGTAGGAATGCTGGGATAACAGACACACCTATCATGTCTGGCTTTACATGGGTTCTGG
GGATTCGAACATAGATTTTCACACTATCAGGTCAGCCCTTTACCCACTGGGCTATCTTCCCTTTCCCCTTTTACAGTTT
TGAGAGGACTAAGTTAACCAGAGCCAGGCTGACTCCAAACTGGCAGTTTTGGAGACGAAGCTTAAGTTTCCTTTTCCCA
GAAATGAGAACCTGCATCCAAGAAAACTGTAGTCAGCCAACCCCAGCTTCGGGAAGCCAATCCAAGGATGCCTTGCCAC
CCTGGAAAGTCCCCAGAGCCCTAACCAATCACAAAAACACCCATACCTCACTGGAGATAGAGTCAAACAATCTAGGGGG
GGGGGAAGGTAAAAGTCACCCACTCCTACCCTAATGGCTTTTAATTGACCCTGAAGAGCACATACCTGTGTCTCCATTC
TGGAATGGGGAGACCCCTGCATGCAGGAAATTCAGCTGAATAAACGCTCTTTGCTTTTGCATGCTATGGAAGACCTGGG
TCTTCCCTCAACAATTCTTGGACCCTTACAGGTTTTGTTTTTTTTTTTTTTGTTTTTTTTTTGTTTTTTTTTGTTTTGA
GACCTAGGTTGGTCCTCAGCTTCTCAGGTGGTGATGGGGCTCTAACCACTGATCCACAGCAGGCCACAAACCAGCTATG
ATTTAGCTTTCTGATGAATGCCGAATTATTTCCACAATGACGGTGCTACTCGCTTTTCCCACCAGCAACGCGTACGGCT
GCTTTTCCTCCTGAATCCTCACTAGGATGTATTGTTGTGTCCTTGATGGCAGCCTCTAACTGGGGTCAGCTCGAACATC
AATGTAGTTTAGTACTTCCCTAATGACTGCAGATATTAAACATTTGCCCATATCTATATCTACACTTGTATGCATAAAT
AATAAATACACATATATACATTATATATTGAGCTTTTGTGCATCTTTTAGGAAGTTTTCTGTTCTGTTTTACAATAGAG
TTTCTTGTGCATGATGGATCATTCTGTTATTGTGGCAATTTTCTGAGTTCTTGGTAAATTCTGCATGTTAATCTTCTGC
CAGATCAATATCCGTCCTCTCTGCCCTGCTGTTCCCTCTGCCCTCTCTGCCCTGCTGTTCCCTCTGCTGAGCCAAACTT
TTAACCCTGGTGAGCTCTCCCATCACGATTTCTTACTCACTCCTAAGCTCTCAGGTCCCATCTGGTCAAGTTGGTGCCT
GTAATTATACTGATAAGTTTCCCCTGAGTTTTCCTCCAGTAGTTTCAATGTTTTCCATCTTACATTAAGGTCTTGGGTT
AAACCTGAGAATCACAAGAAGAAACCAAATACACAATTGTCCTATTAAATAAAGCATGAGGTATGTTGGGGTGTGCCCA
GGACTGGCCAGCTGGTGTCTCACCCTAAGTCGGGATTTGAGAGACCTTTATAGGAGCCTTTATAGGAGCAATAAGGTGT
TCACAGATGTGAGAGAGTTGGCGGTCGTGCAGTATTGGAAAGATACAATGAAAGCTATAAGGGTATGCATTATGTGAAT
GGGGTTGCAAGTTTAGTGTAGGTTGAAGAACATGTTTTGCAGTTCACATCAGCTCTAAGAAACAGGAACTAAGTCTTAA
TTACAAATAGAGACTTTAACTTGCAAGGACTTAACACAGGACAGTCAGGAACTTATTCTTAACAGTCTTAACTGCGAGC
AGAACACTGAATCTGCAAGGTGTAGTTTTCCTGCTTGGGTCTCTCACTGACTTTCAAGGTCTATTGTTTCTGGTTCAGC
CTCACACTTGGATCTGTTTGCATTTGGGTTCTGGGCAGGGTAAGAGATAAGGGTCTGAGTTCCACCATCTGAACATGGT
GTCCAGTTTTATAGTACCCTTTGTTGAACAGGCTGTCTTCCCTGCCAGGCATGGATTTTTAGTTCCTTTGTCAAAGATC
AGATGGCTGTGGTTCTGTGGGTTTATTTCTGGATCCTCTGTTCTATTTCAGCCATCTTCATGCTGGTTTTCATGACTGT
GCCTTGCTGGTTTTGTTGCCGTGACTCTACTGTAGAGTCTACAGTACTGTTATACCCAAGCCTTGCTCTGTTTGCTTAG
AACTGCCTTGGCTGTTTGTGGTCTTCTGTGCTTCCCTAAGACGCCAAAGAATTCTCTCTACTCTGTGAAGTATGTGAAG
AATCCCTTTGAATAGGGATTATATTGACTCTGGAGACTGCTTTTAGGACTATGGCCATCTTAACACTGTTAATTCTGCT
GGGCCCAGTTGCATGGGTGATCAATCGCTCCACCTTCCAGTGTCCTCTTCAGATCCTCCCTCGGTATGTCTTAGTTGTA
TTACATAGGTTGTTCACCTAGGCTGCGCTTAGCCTTATTCCAGGCTCTTTTCTTGCTAGATTCTTGGTTTGTTTTTGGG
CTATATTCCTAATGGAATTGTCTTCTTGATTTCTTTCTCCAGAAGTTCATTATTGATGTCTAGAGACACTACTGATTGA
TTGATTGATTGATTGATTGACTCCTTGTTTTCACATTCGTATGAAAGATGTATATGTGTTGTAGGTATGCATGTGTAAG
CCAGAGATCAACCTCAGATTTCTTTCTCTCTTTCTTTCTCTCTTTCTTTCTTTCTCTCTCTCTCTCTTTCTTTCTCTCT
TTCTTTCTCTCTTTCTCTCTCTCTCTCTCTTTCTTCCTTTCTCCTCTCCTCTTCCTTCTCCTCCTCTTCCTCTTCCTTC
TCTTCTTCTGACACAGCATCTTTCTGGCTGACTTGGAATTTGCTATGTAGACCAGGTTGGCCTTGAACTCACAAAGCTC
CTCTGCCTCTACCCCTGGAGAGATAGGATTAAATGTGTGAGCCACTGTGCCCAGCCTATCAGCTATCTTTTCTTAGGAA
CCTTCAACTTTGTTTTCTGAGATAGGATCTATCACAGAGAAAGGAGCTTCAGTTGAGGAAATGCCTCCATGAGATCCAA
CTCTAAGGCATTTTCTCAATTAGTGATCAAGGGGGAAAGGCCCCTTGTGGGTGGGACCATCTCTGGGCTGGTAGTCTTG
GGTTCTATAAGAGAGCAGGCTGAGCAAGCCAGGGGAGGCAAGCCAGTAAAGAACATCCCTCCATGGCCTCTGCATCAGC
TCCTGCTTTCTGACCTGCTTGAGTTCCAGTCCTGACTTCCTTGGTGATGAACAGCAGTATGGAAGTGTGAGCTGAATAA
ACCCTTTCCTCCCCAACTTGCTTCTTGGTCATGATGTTTGTGCAGGAGTAGAAACCCTGACTAAGACAAATTGGTACCA
GCAGAGTGGGGTATTCCTGTGACAACCTGACCATGTTTTGAGGAGGACTGTGGAAGGACTTTGGTTTGGGCTTGAAGAT
CCATTCATTGTTAAGAGCTCTGTCAGATGTTGTGTAGGAGCTTGGAAGATAATGTTGAGAACAGTGCAGAAGATGGAGG
TCTGGTTTGTGAAATTTCAGAGGGAAAATTAAAGACTCTTTTCAGGGCCATTGTTGTTTTGATTATGACGATTCTGTGG
TTCTGATTAGCTGGGGCTGAAGAATCAGCTGTGATTAACAAGATACCAGAACTACTAAAGCAAAAACTTTGCATTACTG
GGACTATTGATGCTGGTTAGCTGGAGCTAAGAAATTAGCGGTGATTAAGAAGAGACCAGCATCATTGAGGTGACATCTT
CTGGGAAGTGTTTTCTGAGAGCACAGTGGCTGCGTTCCAGATATAGCCAAAGTTGTATCTTGTGCTGTGGCTGGACTTG
GTAATGTGTAAGGGTCACCCAGGTGGTACTGGTTTTGAAGGCATGAAGGAGTTGAGCAGAGCAGCTGAGGCTCGGCACT
GTGAGAGGCCATGGAAGGCCATTGGTGAAAGTGCAGCCTCAGTTGCAATTGATGGCCCAGGACTGAAGGGGTCATGCAG
TGTTTTGGAGATGCCAGTACCATGAGATGACCACCAAGAGCAGCAGCAGCAGTGGAGTAGAGGCATCTGGAGCCTAGAG
GATGACTCGTGTGCGACAAAGGGCATGGCTGGAGAAGTGACCCAAGCCCTTGGAGGAGCCCAGAAGATCTTAAGTTGGA
TCCCAGACATTGGACGGTTGGAGATTGACTTTTGCTTTTGATTGTGGCTGTGCCCTGATATTTTCCCTCTTGAAGGAAG
AAACTGTTTTAGTGGAGCCCACAGTTAAGACTTTTAATTGTAAAAAGACCTTGGATTTTAAAAAAGATGGATATTTTAA
AGAGATTGAAATTTTAAGAATATGTAAAGACTGTGGGACTTTTAAAGTTATTTAGATCTTGGGGATGAATAAGAATGTA
AGGGTTGAGGCTTACTAGTGATGTGTTTGTGTGTCAAGTTGACAAGGGGTCAATTGTACTGGCTAGTTTTGTGTCAACT
TGACACAGCTGGGGTTATCACAGAGAAAGGAGCTTCAGTTGAGGAAATGCCTCCATGAGATCCAACTCTAAGGCATTTT
CTCAATTAGTGATCAAGGGAGAAAGGCCCCTTGTGGGTGGGACCATCTCTGGGCTGGTAGTCTTGGGTTCTATAAGAGA
GCAGGCTGAGCAAGCCAGGGGAGGCAAGCCAGTAAAGAACATCCTTCCATGGCCTCTGCATCAGCTCCTGCTTTCTGAC
CTGCTTGAGTTCCAGTCCTGACTTCCTTGGTGATGAACAGCAGTATGGAAGTGTGAGCTGAATAAACCCTTTCCTCCCC
AACTTGCTTCTTGGTCATGATGTTTGTGCAGGAGTAGAAACCCTGACTAAGACAGGATCTCTCATTGGAATCTGGAGCC
TACAGAATTGGCTACACTGGCTGGCCGGCTATCCATCGAAGTTATCCTGTCTCCATTTCCCCGGTGATAGGATTACAAA
TATGAGACCACCACACCCTGATTTTTATGTGCCTAGGGTGATTGTGTATCCTAGTGCTTTCTGACTTTGACAGACCTGA
GAGATATCTGTAGTGAAGCCTTTAGGGTCTTCTAAGCTAGTGATTGTATCTTCTATAGACTGGTAATTACTCTTTTATT
TGTATCTCTTTTATTTCTTTTACTCGTAAAATTGCTCTAGCTAAAAGTTTAAGTTCTACACAGAGTAAGTGTGGTGGTC
CTTATCCTTATGCCCTTAAATGGGCAACCTTCTCCTCTTCCAAATTTAAGAGGAAATTGCTTCCAATTTTCTCTGTTCA
GTCAGATGTTGGCTATAGGTTTGTAGGATGCAGACTTTAGTACGTCAGAGTCGTGATCTACCTATTTATGTAGGACTTC
TATCATGAAAGGATGTTAAATTTTGTCAAAGACTTTCCTGCATCCTTTGGGCTGATCACGTGATGTTGTCCTTACCCTT
CCTCAAAGCCCTTCCATGATGCTTCTTACATACTGCTATGCGGTGTCTCATTTTCGTTGATTTTAGGACTGCTTGTCCC
TAACTGTACTCTTCAGTATCCATGTGTTCTATAGACTCTTTCCTCTCTCTTGCTGATGATCCCTAGCCTCAGTCCATCG
TGACCTGTTATAGAAATTATCCCAGGCAAAAGAGCATCAGCAGAAGGGAAGGAGTGCAAGCAAGTCCAGATAAGGACCA
GCATTCAGGACAGTCCCACTCTTGGCCACAGTCAGAGAAATATTTCTGTTTGCAGTAGTTAACACATAGCCAATCGAAG
TGCTGCCAGTAAGTGACCATTGAGTGTTCAGCTTTAAATGAAACATCTCTAGCAATGCCTGTTAGCAATCAGGTGTTTC
CACCAGCCTCCCCTTGGGGGCCTTAGCGGTGCTTACATCATCACCTGTCACTCCCAGGTGTGGCACATAAAAGGACCTG
GCCACCCCAGACCCCATCCCTTCCCACCCTGTCTCTGTCTCATCTCATTCCTGCATGCATGTTCTCTCTCTCTCGCTCT
CCCTCTCTGTCTTGTTCATTCTGCTGTCTGTCTATCTGTATCCCTCCCTCATGTCCCTACTCTGAGACAATCTTTCACT
CTTCTCCCTCTCCTAATAAACTTCTGACACTTTATCTGTTGAATGGTGTGATTTCTTAGCTGCACACCTTCCCCACCCT
TCCATGTTCTTCCTATCACTGCCTCCAAAGTCAAGGGAATTCGACAGAAGATGGCTCAGGAAGACTGTAAGAACCTGGA
AACGAGAAAGAACACTGTGAAAAGCTGTCTTCTGGACACAAAATGGCTGTTGTGTCATGAACCCACAATGTCAGCATTT
ATCTACATCCTCCATCACTCTGGGTTCAAAAACATTGCATCATACATAAGGGAGAGACTATAGGCAGCTAGTGGTTACT
GGAGGAGGAGGAGACATGGTCTCCATTGGTGTGTGCACTAGTAAGTGGTCCTAAATGACCAATACCAGCCATGCCAATG
CAGGCAACTCTAGTTGAATGCATTAAAAGAAAGTATGGAAATAGAAGACAGACTTGCTGAGGGCTTCAGGGAGCCAGAG
GAGATAAGAGGATTATGTGGGGATTTTAATCAAAGTACAAAATATATGTGTGAAATAGTGAAGTTTTAAAAAATTATTT
CAAGTGTTTAGGGTTGTTTTTATCTTTGCTAAATTTTACTTTATAACATAAAGTATCTGATTAGGAGTAATTTATGTGA
GTAGCTGAATAGACTGTGCCTCCATCTGCTGTTATATAAAATGACTTATAGCTGTCTGTCCACTTGACCTACAGTGTAC
TTTAACCCTGATTTGGGGGACATTGGAGTTATCTATCACTATTATACATCATACCCAGTAGTGTTTATGAAATTGAATG
CACCAACATTCAATGCATATATGTTATATGTGCATATATAATGTGTTATGATGATGATGATGATGTCCTGCTAGATCCT
TTCTTTGTCTCTTCTTATTGATTCTGGCTTGAGTTATACTTTGTCAGATATGACTACAGTCATTCCTGCTTGCTTTCAG
GTCCCTTTTGCTTGGAATGTCAGATTCCCTCCTATACTTTCAGGCTGTGTTTAAGTTGACACTATAAAATAAGTTTCTT
GCAGACAGCAACTACTTAGATTTTTTTAAAAATTTAGTCAACTAGTCTGGGTCTTTCAGTTGAAAAAATAAGACCTCTT
ACATTTACAGTTATTATTGGAACGTATGTACTAATTTGTGTAATTTGATTGTTCTTCTAGCTGTTTGGTGTAGAGGAAT
TTTAATCCAGCAACATGGCTGCTTTGGCAAGAGACCATATCCAAAGACCTAGATCTATGTGATCTGGCAGGAAAGCAGC
CACAGCCTGAATCACAGTGTGATCCAGCCGGAATTCAGACATGTCCTTTGTACACTTTAATCTCAAACAATAATACAGA
GATGTCTAACTGAGAGGCAGACAAAGTGATGAATCAGAGAAAGATTTGACAGAATGAGTCAGGGATAGGACAATCCCAA
CTCTCATGAGAACAGACAAGAAAGGAAGGCTATATTGGGGTTCATCAAAAAGACCCTTACTCAAAAAGACCACAGAGAC
TGCCACCATTGCTGCAAAACACATGAGGATTTTTATTGATCCAACATGTTGGGGACTCCCCTTTCAGCACACAAGCCAG
AGGAGAAACCCAGAACAAAGAAAGAACACACTTTTTATACCTTTGTAAGGGGCAGATTTGGGTAACAGGGTTCTCATTG
GTGTAGCAAGTAACCCTTTCAGGCATTGGTTGGTTTGTATAGTGACTAAGTAACTCTTTGGCCTAGTGACTCACTTTGC
TTGCCCCCATGGTGGGTTATTATGATTTGGTCAGCAAAGTAATAGTACATTTTGAACTTATGGGTCAGTTATTAACATC
ACAGCTATTAACATCAGGGGAATTCTTGCAACAAGGTCAGGGTGGTTTATGGTCTTTGTCAGAATTCAGTGTGGGGTGG
GGAACAGGAGAATTGCTAATCTTGTTATGGTTATTTCTCTGAGAACAGCTAATATTGTTTTGGCAACTTGTAGGCTCAG
TCATTTTGACTGCTAAAACTATGTTTTTACATTTGTTTAGTCAGCCAGCTTCCTTCTATGGCTCTGCACTTGGCTGCTA
GTACAGTTTGGAAAGTCCTTTTTGAAGGGGGAAGGTACTGGGTGGTCTTTAATTCATTTTGGATGTTACAGCTATTTAA
GAGCAGTGGAGGTAGGGAGGGAGAGAGAGAGAGAGAGAGAGAGAGAGAGAGAGAGAGAGAGTTAGGAGTCAGTGCAGTG
AGTGTAGCGCACTGGAGTTGAGTTTGTGAAGCAGTTGAAGCCAGAGAATAAAGAGGCGCCAGAAGGTTAGACTAAATTG
CCAGAGTTACTTTGAGGCCAAGCAGAGCAATTCAATGAGAAGCTGAGAGACAAGCCAGATCGAATCAGTCAGCCCAGAG
AAGTTTGAGCCAGGACAGCTGAGTTGAACCAGCCAGCCAGTGTTCAGAAAGAGCTAGAAAGGGTGAGCTTATTCAGCAG
TAAGCCTGAGATGACAATGACATCTGGTGAGTAAACTTTACTTTTATAGTTTAGACTATTTTGTTGTTCTTCTTTCTCC
CTATTGGTTTGATGGCTTTGCCCTTGATGGTTTATCCTCAATAATATTTGATTCCTCCCTGAGTCTTGTTTATCTGCTC
CTCTAGTGAGATTTCTCCTTTACCGAAATCTCCTGTCATACCTCTTCTGTGTGTGGGATTAAGTATCTTATACTTAGTT
AACTAATTTAGTTATCTTACACTTAAATCAAATGCTGACCTAATGGTCATGAGCTCTTTTAGTATATGCCACTCTTTGA
TAGTTTCCATTTCTCAAACTGATCCTGAAAGATGACTTCTCTGGACACAGAGGTCTAAGTTGGCAGTTGTTTTCCCTAA
GGCCTGAAAGAGACTAACCATGCACTCCCTCTGGATCTTTAGACTCTAAAGTTCTGAGTGTTTGCTGAGAAGCCTTAAG
TACTAAAGGGATTCCCGTCAGAATCCCATTTGGTGCATCTCTTTCTTATATCTTTCAGGGTTGGGTTTTTTTTTTCCTT
TGTTCTGAGCTCTTGACAGTTTAACTATAATATGATGTGAAGAAACTCTTTTCTGAAAAAGAGGGGGGCAATTAAATCA
ATACTAGCAATAGCTTAACTACACGTAAAGGGCGGAAAGTCAGAACTTTATCCAAGATACTTTGTCAGGAAACTGAAAA
TACCCACGTGTTTCCAGACAGAATCATGAAGAACATTCCCAAAGGCCAACACTCCCCTAAGATGCATCCAGTGGAGCTG
GGAAGACGATGCAAACTCAAGCTGGGAAGAGGATGCAAACTCAAGCTGGGAAGACGATGCAAACTCAAGCCCACGTTAC
CTCCCACTTCCAGTGGAGAACATGATCATAAAAAGGAATAAGCAGTTACCATCAGTGTTTGTCCCTTCCATCAGAGGCT
GAAATCACAGATCTTGGCTAAGGAATACAGTAGGTTGCATCTTCTCCTGTCCTTTGTTCATAGTTAGATCAAAGCCTTC
AGTTCAGCTCTGCTGTGAGCTTTCACAAGGACTTCACTACTCTGGTCTTCTTTTTGTTCAACGGAAGGCACTAGTCACT
CTTCTACAACCTTAAGACTGTACTGATCAGCTGTTTTGGCTCCCCTGACAGTTTGACCAAAATATGACAAGGATGTGAG
CTCCAAGGAACAGAAAAGGAGCCAGGATCAGTCAACCCCATATACTAATGGGTTAGACACCGTGGGACTCATTAAGATG
GTCACATCCTGCTTCTCCACAGTGGACTTCTGGCAGTTGAGACTCTCAAGCCATGTGCACACAAACCATCAGATAGTGG
GTACCACTTGAGTCTCTCCACAATCTCCCTAGACTGGCATGTCTGCCAACAACTACTTCATGGCTTACACATCCCACAG
GAGAATACAGTCATCATAGCTCCAGTAGCCAGAATATGCTCCTCCTGGGTTTGCTCTGGATGCTACACACACCCAGGGA
GTGTCTCTAAATGAACAGAGGTTCTCAGGCAGAATCATGGAGGGTCTATGCCCAAGCTCCGAGCCTGGAGCCAGGTACC
TTTCCCTAGTGGAAACCAATCTGCCAAGCACCCTGGTATCCTGCTTCTCAGTAGACCTTCATTTCCCCCTTGTACGTCG
CCTGTCAGAGCAGCCTTACAGATCCAATGGCATTTGTCAAGCCTACAGAATCATGGCCAGCCACTGGGATGTGGCATTT
CATGTCCAGGATGGCAAAATTATCCTTTTTTGGACCTCAGTCAAAGGGTCAGCAGCATTGTCATTGTTGAAACTCTAGA
CTTATGCTTACTGGTAGATTTAAATTGGTAATAGTCCATAAATGGCTTCTTCGTTTACATTCATGGAGATACTTGCCTA
TAAATTTCTCATAATGTCTTTGTCAGTCTGGACTATTTTTTTAAAAATCAAAAACGATTCTTTCTTGTCTGAAAATACT
TCATTATGGTTGCTATTTTGGGGGTGTTTTTAAAGGGAGTTTTGAGATATAAATTACAGACCATGACATTTACTTTATA
TATGCATACCCTTTGCCATTTTCATAAGTTCTTGGAGTTGTATAGTCATTCCACAATGCAGTTTTAACACATGCCTCTT
ACCCTGTAAAACCCCCCAGACCCAGCTCTTACTCCAGGCAATGACTACATTTATAGATTTACTCTCCAGATATTTTCTA
CATAGACTCACGTTATCCAGGACTCGCAGGTTCTGCTGCTTCATATGGATTTTTTAAGGATTATCCATGCTATAAAATG
AAATAATAGTTTGCTACTCTGCATTGCACTATAATACTGTCCTGGTTGAATATAATATGCATTTAAGTCACCAGTTGAT
GGATACTTATTTAGATTGTCTCTGGGTTTGGGCTATTATGAATGCAGTTATAGACATTCGAAGGCTTTATACCAATATA
TATACATACAGACATACACACACACACACACACACGCACACACACACACATATATATATGAAGAGAGAGAAATACATGC
TTCCATTTTAATTTGCAGGTCCCTAGAAGTGGAATTGCTGTATTGAATACATAAAGAGCCCTTGCACCTTCTTAATAAG
AAAGCAAGCAACCCAATTAAAAACTGCACAGAAGTTTTAAATATGCATTGCATCAAAGAAAATAGAGCATGAAAATAGC
TAACAACCCTGTAAAACTCAACATCTTTAGTCATTAGCAACATGCAAATTAATACCGTAACTCCTATTAGCTTTCCACT
TCTATAACAAAATACCTACGATGAATAACCGTAAGGGGAAGAGTTCGTCTGTGTGCGTGGTGTCAGAGTCTGGACGTGT
TCAGCTATGTTGCTTTGAGACTATGGTGAGACAGATTTGGTGAGGAACAGGAGGACAATCAAAGCTTCTTCTCACTTTG
TGGTAGCAAAATAAACAAAAAGGAAGCAAAAATAAATAAATAAGGGTTAGGGTATCAGTAAACCCTTCAAGAGCTCAAA
GTCATGACGCAACTCCCTGCAACTAGTCCACAGGGACTCATGAAGGTTCCACTACCCCCTAACAGCACCACAGACTGGA
ACCAGGCCTTTAACACCTGGGCCCTTGTAAACATTTCTGATCCAAAGGTTGGCAATGAGTTACCGTGCTACATATATTA
GGACGGCTGTCAAGGTTTGATAAGGACAGAGTTAAACAGGAACCCTCAAATGTGGGCAAGTAAAATGGCTTAGCCATTT
TTGAAAACAGCTTGGTATTTGAAAAAGCCAAACACAAACCTGGCAATTCAACACGTCGCTCTAGGGAAAGTACAAGTGT
TTGAACCAAACCCAACCCCAGGGCCCGGGAGTGGCCTTGGCAGGTGCTCATTTGCTGACTGCACGTTTCACATGTGGTG
TAGCTGTTGTTACCTCTGATGACACACAGAGCTGCCCAGCTTGTGGCTGTGACATGTGAGTGACTTCACCACCACAGCA
GATGCTTCTGAAGACAGAAGAGAACTTAGAAAGGTCACAAACAAGTTTGTGTTAATCTTTCATTCTTTGTGGATTTAAG
CGTATATGATCAAATGGTGACATTTCTTAAGTCCAGAGGAAGGAAGGACATGTCTGTGCCTCTGTCTTAAAGAAAGAGT
CTTTGTCTAAAAGGAACAAAGTCCCCCACCACAGGAGCTTTATAACTAGTTTGTGTGGTAGGGCTCACGAATGTGGGTT
TCTGTGCATGTGCACATGTATGTGGACACTGGAAATCAGTGTTGAATGTCTCTCTTGGTGTCTCTCCACCTTATTTTTG
AGATGGTCACACTGTGCCTGGAGCCCAGTGATTTGGTTAAATTAGACAGATGGCCAGCAAGCTCCAGGAACCCTCCTGC
ATCCAGCTCCCCAAATGCTAGGATTACAGGCATTCACCGTAACACCTGCCTTTTTATGTGGCTGCTGGGGACCCCACTC
AGGTCCTCTTGCTTGCACAGTAAGCACTTTACCAACTGAGCCAACTCCTTAACCCCTAGCGTTTTGAGTTTGTTTTACT
ACATAGCTCAGGTTGGCCTTCAACTCCTACCTCAGCCTCCCAAGTAGTAGGATTTCAAGCATGTGCCATGGCTGGTTGC
CTTAGCTCTTAAAGGAGGTCACTGGATCGTGAAATAAGCCCTATTCTCCCTGGAGAATTGAATGAGCATGTGGCAAATG
GCTAGATCCTGTGGCTTCAGTATTTCCCCCCAAAGCACTGTGAGCAAAAGAAATGGCCTCCCCCGCTGTCTCTAATTCT
CTGTTTCCTGGGGCCCTTGCCTGTGGTGCAGCTTATGGTCTCAAACTGCCTTTATTGCTGTATGCAGATTATACAGGCA
GAGAGTGAAGACCTTACTACTTAGTGTTAAACTGGCATCAACATTTAAAGCTAAGGAAGTTTAGCTTAAAATCTGACTG
GCCATCTCTTGCTGAGCAGCTTGTGAACATCTCTGTGGGGAACCCAACTGTGCTTGTCACCATTTGGTTGATCTGGCTC
TGCGGGGCACCCCATACCAGTCCCTCTGAAGGTCTCCCCCAACCATACAGCTGGTCAGCTGTCTGATCTGATGACTCCT
ACAGCTGCTGTCTCTGCAATTGATGTTACCTGCAGCGTCTCATTCTCAGCTCTCCAGGTCTCCTAGTCAGCATCTCACA
TGGCCTGGGTCAGGCTTGATGAGTTAGAATCATAGTGTGCACTCTCAATATGAAGATTCTGCCCAAGGGCATACAGCTG
CTAAGTGGTGAGCTGGCTTTGAGCCCCTGCCCTCAACCTCTGCTCTCAGATCCATTTCCCTGGGCCCAAGCCCTGAGCC
TGGAGGCTGTCCTCAAGCATTGATGTATCATTAGTCCTGCCATATACACTTGTCAAAGATTCTAGGAGCTGGAGAGGAG
CTGGCATCCACATCACCTGTGTTGGCATCTTCAAAGGTATCTATCTTCCAAGATGAAGGCTCTGTTTCATTAATGTTTA
GAGGAACAATTAATAAATGGATGGACTGTCATCTGGGCAGCTCTCATATGGACAGTCATTAATCAAAATAGATTCAACC
ACAGAAATGTTTTAGCTGAGGCATGAAGGAAAAAGAAGGTTGGCCAGTCAAGCCAGAACCCTGGCCACAGTCTTGTCAT
CACATTTCTGACTTCTACTAGCAGCACACTGAAGGGAGAGTTGTCTGTAAAAAGTCTAAATAATTATATTTAAACAGCC
TTCAGAGGTGAATCTTCCAGAGAACTAACTGAAGGATACCTGTGATGCTTAGAATGAGAAATGTCGCCCCACACATACA
CAAACCTCCGGCTTAGGTATTTGAATGCTCGGTCCCCAACTGTTTGCTATGCTGGGCAGTTTTTATTACTTGACACAAA
CTCAAGTCAACTGAGAAGACAACCCCTCAACACCTCTATCAGAGTGGCCCGTGGGCATTCCTGTGAGGTCATTTTCTTG
ATTAATGATTAGTGGGGGAGAGCCCGGCCCACTGTGGGAAGTGCCACCATTGGGCAGGTTGTCCAGGGCTGTATAAAAA
GGGAGCTGAGCAGGGCATGAAAAATGAGCCGGTGAGCAGCACTCCTCCATGCTCTCTGCTTTGTTTCCTTCTTCCCAGT
TCCTGACCTGAGCTCCTGCCTTGGTTCTTCTCAGTGATGGGCTGTAACCTGTATCCAGAAGTAAACCCTTTTCTCCTAC
AAGTGGTTTTCAGTTAGTTTCATTACAGCAACAGAGAAACAAATTAGGACAGTGGCACTGTGCTCAGATATTATGGAAC
CTTTAGGACAGACTTGCTGAAGGAAGTATGTCACGGGGGGGGGGGGGTTCTTTGAGGGTTTATAGCCTCACACTACTTC
CAGGTCACTCTCTCTACCTCCTACATGTGGTTAGAGATGTGACTCTACTACTGCCATGCCTTCCCCATCATTATGGGCT
CTCCTTCTGAAACTGTAAGTCAAAATAAACTAATCTATAAGTCACTTCTGGTCATGGTATTTTATCACATCAATGAAAA
GTACCTAACCCGCAATCCTTATGTGCTACAGTCTACACATGGTTTGTCTCCTTGAAACTCAATTGAGGGTCATAGGGAT
AGCTCAATGTACAAAGTGCTTCCCACTCAAGCAAGCAAGAGGACCTGAGCTCAAATCCCCAGCTTCCCTGTAAGAAGGT
GGGTGCTGCAACCTCTGTACCCCCAACACTGGGTAGACTAAGGCAGGAGGATACTCAGTGCTTACTGGCCAGCCAGTCT
AGCAAAAATAGAAGTCTCTTGATTGTCTCAAAAAATAAGGTGGGAGATGATAAGGGAAGATACCCAGTGCCAACCTCTA
ACCTCCACACATCCACCATGCACACACGCACACACACACACACACACACACACACACACACACACACACACACAAACCA
AAGTCATGCTGAATTTTTACAATAGTGGTTGTGGTGGTTTGAATATGCTTGGCCCTGGGAGTGTCACTATTTGGAGGTA
TGGCCTTGTTGGGAAATGTGTGACTTTGTTGGAGGAAGTGTGTCACTGTGGATGTGGGCTTTAAGACCCTCATCCTAGC
TTGCCTGGAAGTCAGTCTTCTGTTTGCCTTCAGAACAAAATGTTGAACTCTCAGCTCCTCCTGTGCCATGTCTGCCTAG
GCACTGCCATAATTTTGGCCTTGATGATAATGAACTGAGCCTCTGCACCTGTAAGCCAGCCCCAAGTAAATATTGTCCT
TATAAGAGGTGCCTTGGTCATGGTATCTGTTCACAGCACCAAATCCATAAATAAGACAGTGGTGTTGGGAGGCAGAACC
TTTAACAATCTTGATGCTCTCAGATTGGATTAATCCCTAATTCAGGGGAGACTGAGCAAAGTTCTCGTGAGACTGTACT
ACTTGAGTGCAGTATCTCTAAAGCAAGGCTGCCTCATGTTTGACTCCTTCACACACATTCACTCCTTCCGTGTCCCTGC
TGTCCTCAGACAGTAACAGGAGGACCTTATGGGATGTGCTTGTCCAGTCTTGGATATTCAGCCTCCAGAACCATGAGCC
AAATGAAGCCTGTTCCTTTATAAATTATGATGGTGGTAGCTTGAGTGAGAGCTGCCCCTCATAGGCTCACATATTTGAA
CACTTAGTCCTCAGTTGACAGTGGTATTTTGGGGAGATTATAGAATCTTTGGGAGGTATGCTAGTTAGTTGTTTGGGTT
TTTGACAACTTGACACAAATTAGAGTTATTTGAGAAGAGGAACCTCAAATGAGAAAATGCCTCCATCAGATTGTCTATA
GGTAAGTCTATAGGGCATTTTCTTGATTGATGCTCTGGGCAAGTGTTTCTGCGCTATACAGGAAAGTGGCAGAGCAAGC
CATGAGGCGCAAACAAGTAAGCAGCATGGCTTGCTCCATGGCTTCCACTTCAGTTCCTGCCTCCAGGTTCCTATCTTGA
GCTGCTGCTCTGACTTCCTGACTCCTCTCAGTGATAGGAGTGTTAGCTGAGAGTTTTAAGATGAAATAGATCCTGTGGT
GGTTTAAATGAGAATGCCTCCCGTAGGTCATTTCGATGAGAGGTATTTGAATACTTGGTCCTCAGTTGGCAGAAGCATT
TGAGAGAGATGAAGAAGTATGACCTTCTTGGAAGAGGTTCCTGGGGGCCCCAATCTCTCAGTCTCTCCCCCCATCTCCC
TCCCCACACCCTCCTCTGCCTTGTGCTTGTGGAGAGGTGAGAGCTCTCAGCTACTGCTCTACCGCTATGCCTGCCTACC
CGCTGGCATGCTCCCCACCACGATGGACATGGACTCTAACCCTCTTGTACCCTGAGCCCCAAATTAAACTTGCTTTTTG
TAAGTTGCTTTTGATCTCATGGTGTTTTGTCAAGCAAAAGAAACCCTCAGGCATGAGGTGACAATCTGTGGAGGAAGTA
TGTTACTGGAAGGTTTTGCGGCTTTATAGCCTCACCCATTTCCTGTTGGCTCTGTGCTTCCTGTGAATGGATAAAAGCA
CGGCCAACCAGCTTCCTGCTCCTGCCGCCACAATGCCATAACTTTTCTGCTGTGAGGGACTCTCGCCTTCTGGAATATA
AGCCAAAATAAACACTTAAATTGCTTTGGGCCATGGTATTTTATCACAGCAACAACAAAAAAAAAGTGACTGGTGTACC
TTGCTTCAGGAATTCTTATACATTGTCAGAAAATGAACTAAGACAATATGAGACAAATTCCTTCACATTTTCTCCGAAA
TATGTAATTACAATGAAAAAGATAAAAGCCACACATTAGGAAAGATAGCATTTACATGAAACACTCAATGGAATCTTGA
AAAGTCTTTTCAAATGCAAATCCTAAGCTTTCTTGAGTGACCTTAACTTTTATATTAGGATTTATTCTTGAGACGATAC
ACAATCCCTGTCTAGAGATTTTTAGTGACAGCATTTTTAAGTTCTTCACAGCCCACACTGATAAGACACAAAATAGTTA
ATTTTCTTTGCAGTATTTATTCAGAAATCTTAGCAGTAGACTGTACACAAAAGTGCAATAATTGAGATTCTTCCTCTTT
TCTTTACCAAGTTTATTTCTGAACTTTAAAATAATATTATGCATTGATTTTTTAAAATAATTATTTATTATTATATGTA
AGTACACTGTAGTCGTCTTCAGACACACCAGAAAAGGGCGTCAGATCTCATTATGGATGGTTGTGAGCCACCATGTGGT
TGCTGGGATTTGAACTCAGGACCTTCAGAAGAGCAGTCAGTGCTCTTACCTGCTTAGCCATCTCGCCAGCCCCATGCAT
TGATTTTTTAATCCCATCAAATATTTTTAGACCTGATATTTTTTTAAATGAGTGCAGTGATAAAGCCCATTACCTTGTA
AGCTAATCTTAAATATTAGTGACAAACACAAATTTGAAGAACTTGTTTCTTTCAAGGAAACAGACACCCTAGTTTGAGG
TGGCTAAGGACCCCAGCAGCAGACTCCTGTCTGATGGAGTTGTTATTTGATGCATTGTTCAATAATATTCATAGCCAAG
TTTGTAGAGATTGCATAAAGATGCAGAAATTTTTGTGTAAAGGCAGATTGGGACCACTGCCTACACCCATCCAAGCTTT
GTTTTGCCTCTCCATGCTAGCCCTTCTCTTGTCTCCTCTGCCTCAGTGCACAGAAAAATGCATTTGGGCACTGCAAAGT
TTTCTTGGGGTTCACAGAGCAAGCACCAGCCATGCTATGTGCCCTTTTTCCACAGCAGAAAGTAGAACTCTCTGTCATT
CAGTCAAAAGAGATGGCCAAGGGGTCCAGAGTGTCTTTGATTATCCATAGCCTACAAAAGATGTCACTTGCGGCTGGGT
CTGTGGCAGATAGGGACTTGAGCTGTTTGACCATGATGTCTTAGTTAGGGTTTCCATTGCTAGGAAGTGACACATGACC
AAGGCAACTCTTATAAAGGCAAACATTTAATTGTGGCTGCCTTACAGTTTCAGAGGTTTAGTCCTTTATCATCATAGCA
GAAAGCATGGAGGCGTCCTGGCAGACTTGGTGCTGTAGAAGGAGCTGAGAGTTCTACATCTTGATCCAATGGCAGTCAG
GAGAAGAGACTGTCTCATAGGCAGCTAAGAGGAGGGTCTCTTCCGAAATGGGCAGAGCTTCAAAGCCCACCCACACAGT
GATGCACTTCCTTCAACAATGCCACAATTAAGAGTATCACCTTCCTAAGAGTATCACCTTCCATGGGCCAAGCAGATTC
AAACCACCACACCTAACTTTCTTCCAAAAGGACCATGGAGAGGAGTTCCAGCTCCCTGGGGTTTGACTTGGCTGCCAAA
ACCTTGAGGCTTTGTGAGCCCTGGAATCCACCTGCAACTCAAGCAAGATGCCCGACTTGTCAGACGCAGACAGTAGTTC
CCTCAGAAGCACAAATCTGCTGCAGAAAAAAAAATTTAAGTACCATTGTTTCGATAAGTTAGTTTGTTATCTCAACCCA
TTCACCTTATTCGAGAACTTGTTCTTGAGAGTTTGTGCTAAGGATCTGGAGAGATGGCTCAGGGATTAAGAGCTGCTTT
TCCAGGTGACCCAGGCTCAATTTCTAGTACACACAGTCATCTGTAACTCCAGTTCCAGGGAAACTGACACCTTCTAACT
TTCAAGGGCACCAGGCACACACATGGTGCACACACATACACATTCGGCAAAACACCCATAAAGATAAAATATGTTTACA
ATTTTTACATTAAGTATAAATGCTACCCCCTCTAAACAAGTAAAACGTCTCCAGGGAACCAGAATGAAGAGAGGTAAGT
GGGGTAAGTTTCTGAAACCTTAACCTGGCAGCCACAGACGGCATCCCCAGGAGCCTAGAGGCATAACACAAGTCTAGGG
CTCCACGAACTTGGGCTCTCTAACTGAAATGTGACATTTGTTCCATTGTGACTAGAGGCTGTAACCCACAGTGGTGTTG
CAGTACCTGTGGTTTTAGTTCCAAGAGAAATCACAGATGTTCCCACAGCTCATTAGAATGGCTGCAAACATCCCAGAAC
ATCCTCTGCACGCACGGCTGCTTCAAGGCATGGTGGGTGATAGATCTGCCATAAGGCCTTATTATTTAATGAGTTAATA
GGGAAGCACACATTAGACAGTATCACAAATTTGCTTTTCTTGTGTTTTGATAGCTCAGTGCCAACATAATTGCTGTTCT
CTTTAATGCTGTGCATTTTATTTCTTGCATTTAGTCTTTTTCAGAGAAGTCCCGGGGCTCCACCATAATGCAGGAGGAA
TCCTCAACAGAGAGGCTATCAGTCCTTCGTCCATTTGGGAAGCAGTGAGGATGGTGGGAGAGGATCCTGGCCATTTGGT
GGGGGAGGTCCCCTGGACCTGAGCTGTCTTATAATGTTTCACGTGCTGATTTAGCACATCTTTTGTGTATACACATGCG
TGTTCATGTGTGGGACTGTGTACACAGACATGTGTATGAGTGTATACAGGTGTGTATGCATGTGCACTAGAGGCATATG
TTAGGCAAGCACTTACCAATGCATTGCCTTTCTCTACCTTGCCCAGTCAAGCTCAGAGAAATATCAGAAGGGCTCGCCA
AGCAGTCTGGGGACACAGACCTCCCATTTTCAGAGAGTGGTCTGCAATCCTAGCCATGAGGCTGACCCCAAGTTCCCTG
TAGCCAGGAAGCCACTTGGATCCTGCAGCTTCACCCTCTGTAACTCTGGAAACTGCTCCTCCACAGGAGCAGCACCAAG
ACCACACCCACTGCAGCGAGCTGGAGCTGCCTAGAGCATAGGTGCTTGGGAAATCTCTCTGAGCCATGTTATCTCATCA
GCTGAAGCTTTCCACAGAAGCAACTGCTCCTGAAGTTCTATGATCCACCCTCTGACAGGAGTGTCACACTCCAGAATAG
GAGCCAGAGTAGGAAATACCAAATCCCACCACAACGTGTACTACATTTCCAAAAACCAAATTCATCTTATCTTTTCCAG
TGCTGCCTTGAATCAGGAGTACTGGGGGAGGGGGGAGGAACTTATTAAGATTAAGATAAAATTCAGAACCCTAGTGGCC
CTTGCCTATTATCTCAGTACTTGAGTGAGGTGGGTTTAACCGACGTAAGTTCAAGGGTAGCCTGGACTACACAGTGAGT
TCCAGGCCAGCCTGGGTTACAGAGTGAGAACATCTACAAAAATAAAAATAGAAAATAAAATTCATATATCATAAAATTC
ACTCATTTGAGAAGGGTCACAGGAATTTAACCCTCTGTGAAGATATATGAACAGTCATTGGCTGCTGAGGGAGGGGAAG
AGTCATATTCACCAGTGATATAGCCACTGGTTAAAGTGCCCTTGCTCAAGTAGATAGCTCTCTACCCATGCTCTTTCAG
GCAAGTCTAAATGCAGTAGGAATACAAACATACACACACACACACACACGCACCACTGAAAATAGTAAGAGGACTGTGA
AGGGGTTCAGTGGTAGGGGAAGTGGATAAAATGGGGGAGCTATTATCAAAATAGATCATTCATATATATATATATATAT
ATATATATATATATATGTGTGTGTGTATATATATATATATATATATGTATATATATATACATATATATACATATATATA
TATATATGATGACATATAAGAAAAAGATCCTCCCCAATCTAAAGCATGCAAATTCAATGTTAACTGTGATGTGCTCAGA
GTGGGGCAGTGGTCAGGGCCATGAGTGATAAAATATTTATAATTCCCAAGGTAAAGTCTATACTCTTCCGTTTGTCCTG
CTTCCCCCAACTTCCACCAAGATCTACTGACCTCCTTCTTGCCCGTATAAATTTGTCTGATCTGGGCACATTATATAAA
GAGCCACACTTTCTGTAACGTTCTGTAGCTGACACAGCACGGTAGTTCCCAGATATGTTCTTTCTCTGCATCCCCAAAT
AACACTGCACTGCATGGCTAGCCACATGCTGCTCACCTACTCTTCAACTGCAGATGTCTGGGTTGCTTCCACTGTGGGG
CTGTTGTGAAAACGCCACTGTGAACACGTGTGACCTGCTTTCTGCGTGACAATTTCCTTCCCTTGAGCATGCCTGGGAC
TGATGCTGCAACGTGATGGGCAACTCGACATCTGCTCTCTGGAGAACCAGTGTCATCTGCTTCCCCACATGCGCACCAT
CCTTCATTCCCATCAGCAGTGTAAGCATCCGACTTTTCTCACATGCTCGGCACTAATCAGAGCCCTTTTACAGTGGCTG
GGCAGTGGTATCTCACTGTGGGTTCTAATCTCATTTCCTGGTATCCAGTGATGCCAATTATCTTTTCATGTGCTTTATT
ACACATCTGCATATCTTTTTAAAAGATCCTTTGCTGGTGTAACTTTTTGTATGTGTATGGTTGGTCTGAGCTGGAGCCC
CGAGTCACGAGCCTCTTGTCCCAGCTGAGTCCTTGTTCCGCTATGCCTTGTTCCTTTGCCCATTTTTAATGATTTGTGT
GTTTTGTTTTGTTTTGTTTTTAATTACTGAATAGTTTCCATTCTCTACTACACTTTAAAACTTAACCCCAGGGAAGGAG
GATTTGACTTCAACTTCACGTCTCTTGCCAGGAAACAAGACACTATCCCAGGGGCACTTCAGTCCTGAAAGCTGTGACT
CTTAAATCTCGTTTTTGGTTCATAGAGACCTTCAAGAATCTAACAAAAACCAAATCCGATGCCAGAAAAGCACATGTGC
ACACATCTCTCTTCTCATCTGATCCCGAGGAGTTTCTGGGCCTTCCACTGCTCAGGCATCACCTTGACCGGGTGCTTTC
TTTCCCCAGGAGGACCGTAACTCACTTTTGATGGTCAAAGAGGCAGCATAAAAGGTCCATATTCTAGGTTCCCACAGAG
GAAAGTGTGAACAGTGTGTTGGGGCTACTCTAAGGCCTGCAAGGCCATCACCACCACCCCACCCCACCCAGGCTTTCCC
TTTGGTCCAGCTTCATGCCTTCATGCCCTGAACCTTCTCAGAACCAGATCTTCTGATCACACTTCAGCCCTATGCCTTC
AACCTGCAACCGTACGGAAGCAAACAAACTCTCTATCAGTCAGGAGAACACTGTATACCTGTCACCTAAACAAATCTTC
ACTAGGCCTGTACAAGCGAAACTTCTCTTAAGATAGGCATCTGCTCCAGGCTTGGAGGCGCCAGAAACCTATAAAGTAC
AAGTGTTTTCTCCTCTGCCTTTGCCCCTAAGAAGCACCTGGGCTGCCTGCCTCGAGCAACTGTGTTGGTGCATCCATCA
CGGCCTCCGGCCCCCACTCTCCCTTTGACCCAGATCTACTGTGTGGCTCTGCAAGTCCCACTGTTTTGCTGTTATTATC
CTCCTGCCTCAGCCTTCTGGGCACTGGGCCGCCACACCTGGCATGGACACAATATTTGGTATGGTAGCTCCATCTTTCA
GCCACAAGCTGACAACTCCCTTCTCTCCTCTGTCCCGCACTTCAGTGCTCTTTGTGACAATTGTGTCAACTCTGTCCTC
AGTGATGTACAGCTTATTTATAATATTTTTCTTACCTGTAACTTTTAGGTTCCAGACGCAAGGGTATTCTGCAAACAGA
AAACTCCAGGCACAGCTCTGCCAGGGCTTCCTCATCATTAGGAGAGGGGAGGCAGGGCTTGGAGTCAGGGAGCCCTCAC
AGAACAGAAGGCGACAGGCTCTTTCCCAGTCAAGATGCTTCCACAGTATGCTGTCCCAAGCAGGATTGTAAGTGCTTTA
CAAATACACTTAGTTCATTTTCACAAAACCTCTGAGGTAGGCATTGTTTGCATTGCTCTATCTTACAGATGATAAACTC
AAGTGTGGGGCTCAGTGCAGTGGCTCAGATTCACCCAGCTAAAAGGTGCGCTAGGATTAGGTCTTTAGGTCACTCGGTG
CCCAGAGTCCTTGCTACCTCTTGGCTCTGTTAGAAGGAAGGCTCTAGGACCCTCCACAGACCAGTTTAATTCCCTTATA
AAATGGGGGACATTACAGCTCCTGTGTCCTAGAACCTAGACACAAATCCATCAGGAATCCAGGGCAAGAGGCATGAACT
AACTCATCTGACAAAGTCTCTGCTGCTCCCCAGCCGTGGCAGTGCTGTGTTCTCAGTGCTCCTCGGCATTTCATAGGTG
AAGACAGGCTGCCGCCCGGGGGACAGAGGATGCTCCAGGACAGACTGGGCAACACTAGACCTTGTCTGAAATAAACTTC
CGATCCTTTCCATCTCATCTGGGAACTCTGGTCAGTGACCATTTATAAAACCTGCTAAGAAACAAACTCACTAACTTTG
GACAACTTTAGGTAGTAGGAAATCCTTTGAAAAAGCAAAGTTGAGGGTTAAACTGCAATCGAATGTTCTGACAAATTTA
GACAACAGACCATTTCAGCTTTTGTAAGAACACAGTATATTTTTAAGGAAATTCATCGCACCTCGTGATTCCTTGAGTG
CTCCCAGGGACTGATCTGTCCCTGCTCCTCCCAGTGATGACATCAGACAGAAATCTTTGATCTGAAGCTTGGGAGACTC
TGAAATTCAAACTCTTTCATCCCCACAGGTGTAGCATCTTGGAACCTGAGCCCTTTAGAAACCTAACTTCAGGAGACCC
TCTTAACCCCAGTATTCTCATTCTAGAGAGAAGAACAAGCGAAGGCTGCTGGTTAGGACTGTTACGTGCCCACCATGGT
TACTGAGTGCCCCCATATGGGCAGCATGCTGTCCAGTCCTGGGGCACAGAGAGCAAGGAAGGCAGCAGCTGCGCCCAGT
GCCGTGATGGGCACACTGGGGATGCTGTGAGAGTGTGAGCCGCTCCCTGCCCCTGGCCAGTGCATACCACGTGGGATAG
GGCGGTCACGAAAGCAAAAGAGCAGTAACAGACTGTGGGAAGAAGACAGCAGGAGGTCAGCATGTGTCGCAGCATAAGG
GCAGTATGTGGGCATCTTAAGGACGACATTTACTCCTGGGTCTACCTCCTAGACGGGCTTGTGCGGGGTGTGAGAAGCC
CCTGTCTCCAAGAGTCATAATCCAAGTAACAAAACCTTCTTAGAGGGTGAAGAGCCCACGGAGGAAGCCCTTCAGGAAA
CCTCATGTGAGCAGCTCACTGCGGGCAGCACTGTGCTGTGCACAGGCCCAGACCATGGAGACCCTGTGAGCATGACCCA
TCATGGGATTTTATTCTGAGTACAGTAAGAAGCCTCTGTGGATTGCAAAGCAGGAAGCAGTAACATTGCATTCATGTTC
TGGACACTCTGTGGAGGTGAAGTAATTGGTGTTGGATACCAGAATGGTACCAGGAGGGAGGCCACGCCTAGATTCTATA
TGAGAGTGGCTTGTGTTAGCGCAAGAGCGCAGTGGGTGGATCTGAGAGAAGCAGAAGCCGCAGGGCCTCCCAGTGAACT
GGGCAGAAGATGTTCTAAGGAGCCAGGAGTGGCATGGAGGAACTGGCAATCACAGATTTCATTTCTGAGGTGGAACCTT
GGCCAGAGGGAAAGTCAAGAGTTCTCTCCTCAGACTACCAAATAAAGGCATCTCACAAAAACAGAGGTATGTCTGATTG
CTTGACTATAGAGAACACTGGGCTACACATGGTCATAAGAGCTTGGGAACAGAGGGAAAGTTCTAGAGCAGAGACCAGG
CAGACAGAAGGGGGTCAGCAGCACCAGGTCATAGGAGTCAAGGGAGGGTGGGAAGAATGTGTGGAATGATGTCCGGAGA
TGAGGACATTAGTAACCTCCAAGGCAGGACAGCAGCTCCAGGGTGAAGAGGGCTCAGAAAGAGGAGACAACCCAGAAAC
CATCACCACAGCCACCTATTTCTAGAAATGTTTGCCAGAGAGAAGCAAATATATGGAGTAGATGGGCACTTTTTCTGTT
TCTTTTGAAAAGAAGAATCCCGGGATGTTTACGTGCTGATGGGAAAGACCCTCTAGCAGGGAGAATGGGAGGAGCTGGG
TCCTTGAGCTGACAAGTCCAGCATGTGCCTTCTCCTTGCCCTGACTGCACGGTCCACTTCTGCACTGGTGTGGCTCCTA
TGCGCCTCAGCTGCCTAGACCCCTGCTCTCCTCTGTCCATGCCCTCCTTGGTTGTCAACAATGTGGCTTCAAATCCTCT
CCCCCCTCTGACCTCTCCTCAGCATGCGAATGCTAAGCATTCGGACATCCCATGAGCTGCATACACTGAATCCAGCTCC
AACCTATACAGTCCTCTCTTTGTGAATGTGTGCAAAAGCCTCCGTGCCAAGGACTCCCCATGGCCCCTTGACACACTTG
TAGCCAGACTTTGCACCTAAAAAGGTGAGAACGCCTGAGAAGCTAGCTGAGCAACTTCACAGCCATGCTTCCACTGCAT
ACTTCCTTTAAGGGAAGCTCTGTAGTTACTCTAGTTTCAAACTGTATAGTACAATCTTTATTTTCAGTAGTACACAGCT
ATTCACATGTAAGTCGGAAAGAGAAAGCATTGCTCTGTGGTAATAAACATTCATGTGAGAAAAGTCTGCAAAGCGGTTT
ATTCTCACAAGTTCCTATGGAAGCAGTCCTCCACACAACGCAGTGAAAATACCCAAGTGCCCAGTCTGCAGACTCCTCA
GTGCCGCCCTTGCGAGCATTGCTTCATTACAACTTTATAATCCAGTCTTCCGTCTGTGGCTGTGCAGGGCTACACGAGG
GCTGTGAACTGCCTCACCGTCTTCTCACTCTCTCTTTTCAGTTCTTCAATGTGGGCGTCCAGGCGCTCTAGAATATGGT
GAGACCTCAGCTCCTCCTCTTCCAGAAACCTGGTAGCTATCGATCCCAGAGGAGAGGTGGGCAGCGAGCCCTGGAAGAG
GGACAAATCATGATATGTGTTAGGAAGCAGGAATGAATGCCCCAACATTTTGATATTTTCTGCCTAGCCCTGTTAAAGG
GAGAATTTTTCCTGAGGAGATATTTTCGTCTTAAATCCAAGAAAGAAAAAAAGGCAAGTTCTGAAAACATAGCTCCACA
GAGCTCTTTCTTTCTGACCCAAAGAGTGTCTGTGTAAAGTGTATAGGAAATGTCGTCAGTTGTCTGAGAGTACCTCGGC
TTAGACTAGAAATGCCTGCCTGCCTCTTACTGAAGAATACAGCCAACGCCTGCTATTTCAAAACTCTGAATGTTACTCT
CACTTTAAAGCACTGAAAATCTACAGGAAACAAATTAAATGCCAGTTAAAAATCAATATTTTTCCCTTTCTTAAAATTC
CACTCCATGGCCTTTGATCTGCAACTCTCAATTCTGGTGATACTTCAGATCTCAGATTTTTGAAGGTAATACAGGAAAT
CTAGCACACGCAGTTTGCTCTCATTTAATGTTATACTTTAGAAGTCACTTTAAACGCTGAGTTAGCACAAATCATTGCC
TCCGGGGGAAATAAACACACACAGTATAGACATATATACACCCTCTTGTCTCTATTTGACAAAACAGAAATGAGTTTCA
GAAGTGAGAAATGACTTTCCCACGGCTACCACTGTAACTGGTGCTGGAATCAAGGCTCTCACACAGCAGGGTGCTGCCC
GCCATCACCACATGAACACTGAGACTGGAACGAAACACAAGGCCACCTTTGCTCCCTCTATTAGGAGCGTGGGAGCCAG
CTCTCCTGCTTTCTGTGTGTCCACATGTGGGTACAGCAGGGCCACAGTACAGTTTAGTGGGCAGTCAAACCGGCAAGTG
ATGAATAATAAGGACCAACCGTGTTGGCCCTGTAGAGCGCAACCATCAGATACCTGACTTCTGAAGGAAAAGCGTGATG
CTCACACTAGCTGGAAAAGGTGACATTTGTGAAATCACATCATCATAGGGCAGGGAAGGCTGGCTGCCAAACTTAGAAG
AAAGGTACTTTTCAGGGTGTTGATTTTTTTTGTTAACTATGTGTAAGTTAAATTGGACATTTATTTTGAAAACTTTAAG
AAAATCTTTCCATGGAATTGCTGACACATTCAAAAATAAACAAACAAACAAACAAAAAAACAAAAACAAAGGTTACTAA
ACTTTCACAAAGACACACCACAGCTAACAACAGGCAATGGACAGCCCAACACCGGCTTCACGATGGAAGGTAAAGAACA
CTGGAGCCTAGTGGTGAGGGTAAGGCGCCATCATAGGTACTCAAATCATGCTCCATTCACCTTAGATGCACCACCCTCA
GCCCCAGGCCACCAGCTCCTCCCCAGCAGCCCCTCTTTTCCCTCCTCAGTCTCCATGCTTGAGACAGAGCCCTTGATTC
AATCCCTGCAGCATGGTCAGCCCTCTTGGGAAAGGCAGGCAAGAAGGCGCAGGCTAAAGTCAGTGAATGCCTGCACTCA
TATTCTAAGTATTTTAGAACCTGAATTTGAAGCTGTCTTGGTTGAATGTGCCTTCACAAGCAAAACTACAAATGTGAAA
GTGATACAGGGTACTGACTGGGACCCAGTAAGAAAAAAAAAAAATCACCCTCTTTAGCTTAAAATTAAAAATAAAAATA
AGTATTAGCACGAGAAAGTTTTCATACCGAAGATACAAACTCTTCTTGGTCATTCATACAGTCAGACAGACTGTCAGAA
AAGTTGACATCATTTATATCTAGCACTGAAGGCAAAGAGTGAGCCTCAAATAGAAAGTTGGAAGGCAGAGAGACGGTGG
AGCTGCCGGGGCTGATGTGAGAACTCAGGGGGCTGGGACCCCTGGCCACAGGGTCTATGCTATCCAACGTTTGTCCTCG
AGGCGAGTGGTGACCTGTTTTGTACTGCTCGGTCTTTATTCCGTCATGTCTGTGCTGGCCATGGATTGGCCTGGATAGC
AGATTAGAAAACAAGATAAATGAAAAACTGTAGCAACTTTTCTTTCCAATAAATGCTGAAAGAAGCATGTTCATAAGAG
GCTATGCGACAGGACTCTACTGCTTACAAATGAGCAGTCCACTCGGAGACAAGCGCTCCTCGGGCTCTGCTCTCATGCT
CCCAGCATAGCAGCGGCCAAACATGCTGCCAGTGGGGTGCAGAAGGAACAGGGCCAGGGCACCCCGTGCACAGAGAAAG
CTCAGAATGTCACCGTGGGTGAGAGTGGGGTGGGGATGCTAACAAAGGCTTCTGGTAGGAAAACACACAGGCTACACAG
AACAAACTCAAGTCAGTTGGCTTCTCATTGAAATAACCTTTCTTATGGTAACAACCTAAAATTAATTGATCAAAAGTGC
AATGGGGGGCTGGTGAGATGGCTCAGTGGGTAAGAGCACCCAATTGTTCTTCCGAAGGTCCAGAGTTCAAATCCCAGCA
ACCACATGGTGGCTCACAACCATCTATAACGATATCTGACGCCCTTTTCTGGTGGTCTGAAGACAGCTACAGTGTACTT
ACATGTAATAAATAAATCTTAAAAAAAAAAAAAAAGAGTTGGCAGGTTCTTTGGGCTGGAGAGATGGCTCAGTGGTTAA
GAGCACTGACTGCTCTTCCAGAGGTCCTGAGTTCAAATCCCAGCAACCACATGGTGGCTCACAACCATCCGTAGTGGGA
TCAGATGCCCTCTTCTGGTATTGTCTGAAAACAGCTACAATGTACTCACATAAATAAAATAAATACATTTTAAAAAAAA
GTGCAATGGGTGCTTTTTATAGAAAGCCACGTTTTACTTCTTATGGAACAGGTGTGATGAAGGTAGTGGGAGGCAGCAT
GAAAACTCAGGAACAAGCTGCTGTTCACACACATGTACACTCAGGGTGCACAGAACAAAAGCTGTGATGAAATAGGCTG
AGCCACCTGAGCCTCTTGTGCACACTGGGCAGAGTTAAACCAGAGCCTGTGATGACTGCACAGCTCTAAGCCAGAAAGT
CTTCTCAAAGTAGGAAAGCAGGCCGTAGCACATGCACTTCAAAGAGACTTTTAAGAAACTTTACTTAAGGTGCTAGAGA
GATGGCTTAGTAAAAGAGCACTGACTGCTCTTCCAGAGGTCCTGAGTTAATTCTCAGAAACCACATGGTGGCTCACAAC
CATCTGTAATGGGATCCGATGCCCTCTTCCGGTGTCCAAATACAGCTACAGTGTACTCACATACATAAAATAAATAAGT
AATTCTTTAAGAGACAGAGAGGGAGGGAGGGAGGGAGGGAGGGAGGGAGGGAGGGAGGGAACTTTACTTAAGCTCTGCA
TTCTTGAACCCGGCAGCTCTGCTGTGTGCTGGGTTAAAGATCTCGCCATCTTTTTTTTTGCACATGTTAGTAGCATGCT
CCAGCTCAGATTTGGTGCTCATCAAATCTTTCTGCAGTTGTTGATTTTCAGACACTAATTTATTTAAGGCCTCAATATA
ATTCTCCTGAAGGTCTGCTAGTGAAATCTCTTTTGCCTAATAAAAAAGAACACGAGAATCACTAGTATGTTTAAGGGTA
ATTATGATCAACCTACAACAGTGTTGGCTGAGATATTTTCTGTTTTAGTTTTAATTATGGCTTTACTTAGAGAAAGTAA
AGATTTGTCTTTAGCCCTCTTAAGATGAACACAGGAAAAGCATCAGTCTGCCTAGAGTGAAGGAAGGGACAAAGTTAAA
GGCAGGATGGCTTCCTTCTGTACATAAGCATTCAAGGCATGGAAACAAGGCAGAGGAAAGCTTCAAGAATCAGACCTGG
AAATCAAGAAGAATGAGGAGCCCTCTCCTTACACACAATACCTGTGGGAGGAAATTAAAAGAACTAACTCAAAGAAAGT
AATTTAAACACAAGAGTCTAAAGAGAATTCTGGCCAAAAGGGGTAACTTTAAAATAAATACCCTTCCATCTTAAATACT
TTTTGGCATGTAGTTTGATATGAAGTTAGCAACATAAAAGCAGGACTATTTTATGTGGCAGGAATTTACCATGAACTGG
TCTTAGTTCTGTGATCATATATCTTATTTCTTGATAATACACATCTACTTTCTTTTTCTAATTTTTTTCATTTAATTAG
CATTCTATAAAATTATGGTGATCTTTACAGTTTAACATATTTCATTTTCTGTAAAGAAAAATTGTGAAAGTAAGCTAGT
CAATAGAGTTCTGATAAACATAATTTGCCTGTCAACTCACAAGTGTGTAAATAGACCTGTAAACTAGGATTAGTAGCTG
TCCATGTTTTATAATATCTCAGAGAAGTGTATTCACAGGACAAGTGCTACACTGTCGACTACCTACCCACCTTTTTATT
TCTAAAATGCAGGGCTACGCTATGAACTACGACTCATCTTTAAAGTGGCCAACATTAATCAAAGCTGCTGCTTTCATTA
AGATAATTAGATTAATTTGGAAAAGTAAATTCACCTTCCTGGGTGGTAACATGCAAAAAGCATTTCCTAGTAATTATGT
TTTGATAATCTGTACAGATATTCATACTGTATGCAACTCTGAAACCAGAGAAAGCTAAAGCTGACAGGATTTTTACAAC
TTTGATGTCTGAAATCATTAAACCAGCTTTCCTTAACCTACACCCCTTTAGCAAATGTCTATCTCCAAAAATATTTAGA
TTACAATTCATATCAGTAGCAAAATTATGGTTATGAAGTAGCGACAAAAATGATTGTATGGTTGGGGTCACTACAACAT
GAGGAACTGTACTGAAGGGTCGCAGCGGGGGGAGGGTTGAGACCCCTTGCACATAGCTAAAAAGAAGAGAAACTTGTCT
TTTCTGTGCATCTTTCTGAAATCTCCGAGAGCTATTAGCTTACTAACAATTCCTCCCGGTGCCTGAGCGGGAAGGGCGG
CATGACACCTGGGGAGCAGGCATGCACTCAGTGGGCAAAGAGGGTCAGCGCTCCCACTAAGGTTCAAGAGAAGTTCAGC
TCCACAGAACCCTGCACAGTCTGCCTTTAGCATCTCTGGAATCTCTCAGTTCTTTAATGTAAACAGAGCCACTCTAATA
CTGACAAATGACACATTTTTAAAGAACACTATAGTGTAAACAGATTAGTCCACAAATTACTACTCGCTCGGTATTGGCT
TTATCAGGGCATACAAAGTCATTTGTTGGCTACACTCTCACTCACTCACCTACCTGCTCATCCATTTACCCACTCACCT
ACTAATCCATCCACTTATTAGCCATCCATCTAGTCATTAAGACAGCCAGCTGGTTAGCCATGTGCCCCAGCCCCCACCT
CCATCCATCTTCCACCTCCTTGACAAAACCTTATAAAATATTTAGCTATGGGATTGAGAAAACGATTCAGTTGGTAAAG
TACTTGCCGTGCAAGAGGACCAGAGCTCAGATCTATAACACTCATGTAAATACTGGCCGAGTAGACACAGTAGACTGCC
AGTAATCTTAATACTTGGAAAAAGGAGACAGGATCTCTGGAGCAAGCCCAATTTGCAAGCTCAGGGTCCAAGTGAGAGA
CCCTGCCTCAATACAAAAGGTAGAGAATGATAAAGGAAGACATTCAATATCAACCCCTAGCTTTCATACATTCATACAC
ATGTACATACACATGCGTGTACACAATCACCCACCATGCACACAAAAATAAAATTATATTAAATATTTTAAATGTTTGG
CTATTGTCTCCTCTATTTTTTTTCCCATCCATGTGAGTTTGTGCTTAAAAAATTTTTTTCATTACAGTATCACAGGGAT
GTGGAGTAGCAGAAAAATTAAATGAATGTTTCCATCTTAAATGACAGCATATTTCAGAGTACAATCACAAATCAACCAT
TTAAGTAACCAGTTTATTTCATAATACCTGCTGTGCCCATTTCTGAACCTTTACTCCCATAAGGTGGGCTCTCTGGGGA
TGCAAACTGCTGAGGACACTGAAAAATGTGACTACTAATATTCCAGGTGGAAATACCAGGAGGACAGTGAAGAATATGA
CTATTAATATCACAGGTGGAAATACCGGGAGGACTGGGGGTGTATGGACTTACAAGCTCCATGTCCATGGCTCAACTGT
ACTGAAGGCAATCTCTATATGCATGGGAGAGCCTGCTGTTAAAATACTTGCCAAGAGAGGCCATATATTAACTTTCTAA
ATCATTATCAAGCTTATTAGCAAGAAATTTTTATTTTTAAAAATTTTGGCTATTAGGGAAGTTTAATAGCTTCCCTCGC
TCACTTGAATTTAAATATCTACTTAGTTCTTTGCCCTTTTCCGACTGGGTCTCTTTCCTATCGCCTCTGTAGGAACTCT
TACTTGCTGACACTTGGTTAGCCTGTGTCTACTTCCAGGTTACTGCTCCTTTCTTTCAAGCACCAGTGCCGCCAGCCCC
GGGCCTCAAGCTAGGCGAGTGCTCTCCCTCTAAGCTCCACTCCCAGCCCCTCTGCTGTTATTTTTATTCTGAGACAGGG
TCTTGTCCAGTTTGGCCTTGGACTCTCGCTCCTCCTGCCTCAGCCTCTCGAGAGCTGGGATTATAAGCATGAGCTGCCA
CACTTGCTTCTATTATCAGTCTTTTCAATGGAGCCTTTTGCCATCCAGAAATTTTACATTTTTATGTTGTAAAATCCTT
TGTGGCTTCATGATGCTTAGAGAATCTTGCCCATCTGAAAGATTATGAAAATAGTCCCCCATTTTCCTAGTTGAAATAA
AGTATTCTTCTAGCCTTTTGCGTCTTCACTCTCGGCCTCCATGACTGTGAAATTGGAACTATTAGGAGCAAACTGGGTG
CCTTTTGTGACTATGAATCTTTTTGGAAGCAGAACACTCCCTGGAAGTTCAGAAAAAAAGAATTCAATCTTCCGACTTT
TGACCTACACCAAAAAAAGATGGATCAGAATAGATGCATCATTTTTTAAATTGTAAGACTTTTACAATATAACTTCACA
CTGTCCTGTTCTGATTTCACAGTAGCTTATATGTATTTCTCATATTCTCCACTAAATTAGTATAATGTCTTCAGAGTCA
AACACGTGATCAGTTATCCTGTGTGCAATTTAGCACTGAGTATTCTAGAATAGTTGAATAAATGAACTATGTATGCTTC
ATAGAAATAAAATGAAAACTAAAATACTTCAAGGATGGTATGCTTTATAATAAAGACACTGTGAGCACAACAGCCTAGC
ACTAAAAGCAGCGCCCAGTGTCCACTTCACAATGACTGCCAGCACTCTGCTGGCACTTTACAGATACTTCCCTTGCTGA
GAACACAGACTATATATCCATCTTGTCACACACAATAGGAAACTATCCTACTGCTTCAGCATAAACATCCTCTGGCAGG
AGGGCACCTGGACCTCATTTGTGACTGTATCTCCTAAGGGACTGTTCAAACTAGTTAATTCTACCTTCTTCTAGCATCA
ATATTAACAGTTACGTTCAAAGGCTTCAAAAACACAAAAGCTAAGTGTCGATGTGTGTACCTCATGCAAGCCCAGTTCC
AGCTTCATCACTGTTTCAGAAAGACGATGATTTTCTAATTTGAGAGATTCCAGCTGACTTAGAATCTCCTGCAAAAGGA
AAATCGGGGGACTCACATAAATCAAATATATTTTACAAAGCTTTTCCACATCATTTTCTCTAGTCTACTTAGTCCAGTC
TACATCCTCTATCCATATAGATAAACTTTTAAAATGTGTGTAGATACAGGGTGTTGTGTGTGTTGCATACACATGAGAG
CACAAGTGTGCCTGTCATGTGTATTTGCAGAGACCAGAGCCAACAAGTTCTCAGAATTCAGCTCTCTCTGCTGTGCCCC
TCCCCTGCAATGCTAGGCTAATCAACAGACCATGAAGCCACGCCTGGCTTCTATGTAGGCTCTGGGAATCCCAGCTCAG
GTCCTTATGCTTGCAGAGAGGGGCTCTCACTCCCTCAGCCGTCTCCCAGGCCCCACATATGCTTCTTAAGAGTGTCAAA
GGTGTTAGCAGGCAAAGCACTGTGCATGTGATTAAATGTCACTCAGTAATGAATGGGACTAAGGCACAAAGATTCAGTC
ACAGAAGATCTTAGACACACCCTGCTTCCAGCTCGCATCAACTTTCTGCCACCACCACCACGTGGGACTGCGCTTGCAT
AAAGCATTCATTTATTTTCAGCCCTCACTCTGCTGAGCTTTTCTGCTAGACTATCTCTATGAAAAAATTCCTGCCTCCT
TTCATGGCCTTCTTCCTCAGCAGGCACAGTCTGGGAAACTCCTTCACTGCCACTTCTTTTAAAGTTCCATCCCACTCCT
TCACTCCAACTGAGAAATCTGCGAAATTTGATCCAAGTCACTCTTAGGCATACTCTATTGCCTTTTCACTATAACTGCA
GTATCTGGGGAGAGGGATTTCAAATGCCACCTTCATCCAGGAATTCCACAACTAAATGGCCAACCCTTGAATTTCAGGA
TTTCCGTGGAGCCAACTAACCAATTTTTCCATATAAAAGTTCAGGCTTTATCTGAAACCAAATGTGCCAAACTAATGTC
TCCTCTGCTAGCTTCCCTATCCTGGGCATCTTTCATAGAAATGCATCATCTTCCACAGCACTACAACCATTCTCTGCCT
TGTTACAGGGCATTACTTGGCTACTCAAGCTTCATCCCTCAGTAATAGCTTCTTACTGTCCTTTGTTACCCTAGTCTAA
TCAAACACAAAGCCTAGCCAACCGTGCAGCCAAGTATCTCCACAACGCATCCATTCCCCATTTCCCTTGAATGGTGGCT
CCCCACCCGCTGCTCAGGCCACATCTTGCAGTTTTACTAGCTCTGCATGGTCTCTTCCAGCTTACTCATTCTCCTCTTG
CAGGAAGAGAGATCTTTACTCTAGATAAGCAACTCCTTGGCTGTGCTCCCAGTACCTTGCCCACGTACCACTAAGGTAG
TCATTTTCCTTGTCTGTATCAATCTGACCTCCCCACCAACCCTCACTATCCTGCTTGCTAACCAGCCCAAAGCAGGTGT
CTACCACTGAAATACGGAAGTTATTCCTTGAATAAATTCATTCTTTAAAAACAAACAGGTAAATATGAAGTGGAGCGCC
CTGGATGGACTTTACCTTATGCTCTACAGCCTTTTCTTCTGCCTTCTGCATCTCTGCCTTCAGCTGCTTTTCCATGTCT
GAGGAGGAACACTTGGCAGAAGCAATAGTGATATCTCGCTGGTGCAACTCCCGAGTTAGCTGGGAGATTTCCATCTTCA
TTCCTTCCAGGGCAGAACTGTAGGTCTGATCAGCCTGCAGAATCTGTTCCTTCAACTATAGAGAGGCACAAAAGAAAAC
ACCAAAAGTTCAGAGAAAGTATTTAGGATTCAGGCATATAAATCTACACAGCAGTAACTCTGACACTTAGGAGGTAGAG
GCAGAGACAGAAAGACCCCAGGATTGAGAGCAGCCTGATCTGCCAGTGATTGCAAGGCCAGATTGAACTTCCAGGAAGA
CCCCTTCTCAACAAACAAGGTTTAGAGATGTAGCTCAATGGTAGAGCGAGCACTGGCCTAGCATGTCCCTAAAGAACAA
GGTATTCAGAGAGAAGACACCTGGTTAATGGCTGCCAGGGCAGGGTTGTCATTTTCTTCAATAGTGTAGTTACTGGTAA
ATTGCTCCAGTAGATAACTCCCTGCCAACGTTCATGCAAGCAACCCTAAACAATGTAGGCCTAAAAGAAAGACAAAGAG
AAAGAAGGAAGAAGACAAGATAGGAAAGGGAGATCATAGAAGGGAATAAAAGAGGGCACTGGAGATGACTATGATATAA
TATACAGTACATATATATGTATGAAACTGACAAGTAAATGTTTAAAATGTACAGAATTTAACAAAGAAAAATATACACA
AAATTGATAAAATAAGGTAATGTCTATCTTACCTTAAGACATGAAATTTTTCCTAGATTAGATATACAGAAAATCATCA
ACTACAAGTTATCAACTTCTATAACTACTGTGAAAGTCTGGTATTTAAATTCAAAATCATGCATTAGGTCTCAGAAAAA
CATGTGTTACTCAAACACTGGGGTCAATGGCAAAGTCTATGTCTAATTTATGTAGTCTCTAAGACTTTCATTCTTTTCA
CAGCTAAAAATTAAAATTTGTTTGTTTGTTTGGGGTTTTTGTTTGTTTCTTTATCTAGTTTTCTGTTTCTCAGGAGAGA
ACTAACAGAAAGATAAGAGGTAGAAAGGGGCAAGAGCCTTTGAGGAAGACAGCTCAGTCTATAACATACCTGCAGCAGG
AGCAGAAGGACTTAGTCTAGATTCCTAGCCCCCACATAAAATTCATCCTGGAAGGTTGCATACATCTATAATCCCAGCA
TGGGGAGAGGGACAGCAGATAAAGGTTGATTCTGCAGTTCAATGGCCAGCTAAGCTAGTCAGCCAATGAATACTGAGTT
CAGTTAGAGCTGCTGAGGCAGGTGAGTGTGTGGGGAGAGAGCAATAAGCAGCTACTTAATATCTACTTCTGGGCTCTAC
ATACACATACATGTGTGCACCATACACACACATGCACAGGAACTAGCAATAATCTCAAAGTAGTGAAAAACACAAATTA
AGCCAAGTATGGTAACAGATGTCTCTAATCTCAATATTTAGAATTCAAAAGACCAGAGGATTATGAGTTTAAGGCCAAC
CTGGGCTATAAAACAAGACTAATCCTCTAAATAAAGCCTCTAAAAAAATTAATTTATTTTTGGTATTTTGAGACAGTCT
CATCTAACCCAGGCTGGCTCTGATCTCCTGTCTACACCTCCCAAAGCTAGGATTAGAGTACACCAGCACACTGGGGACA
GGACCCCTTAAAAAAAGGCTCTAGAGTACCAACAGCCAATGAGGACATTAACTACTTCATAGCACAAAGGAAGCCAATA
AGGAAATTACAGGTAATATAATGTCTTTTTTAATAAGCAATATAATTGTTTAGAAATCATCATGAGCATATTTCACAAG
TTCCCCACATGCACAGTATCCTTTGATCATCCATCCCTATCATCCTCTCATATCCCTCCTCCTCCCACTGAGTTCCAGC
AAACTCTTCCACTGACCACCCAACACATTTCCCTCACTGAGCACCTCCTGCCCAGCTCTGAGCCCAGGCTCTGACGGAG
GCTCTCACCTTCTTTATCTCTGTCCGGTACTCATTGTTATGCCCTTCCATCCTCTTCAGCTCTTCGTATTTTTCCATTA
GTTCTTGGCTCAGCTGTTTGCATGTTGCACTCACAGATTCTAAACTGTATGGCAGAAATACAACCATATATAAGTAAAA
TTCCTGAAACCCATAAGATGGATTATCTAAGTGTACCCTCTTGATGCCCAAGATACCTCCTACTAACACCTTAAACCTC
ATACCCGTCACGAGAAGTCATTTGCAATTCCCCTATCACTAGATGCAGCAAACCCATCTCAACACTGAGGTAGTTTTGT
CTTTCGTGTTTTCCTCCACTCCCGCCCCCTGATTTAAACTTTACTGGCTACAGAATTATAAAGACACCAGCCACTTTAC
TAACAGTTTTGCTCTAGCTGTATGGGTTTCTCTAGCTCTATGAGAGAGGGAAAGCCACCAAGAACTGTCACAACAGACT
CACACTTCATCACCCTTAGCAACACACAGACACACTCCACACTATACAGAACCACACTATACAGAACCGCAAGAACTCT
TTGAAGAGTCCCCAACTCATTGTTCTTACTCAAGACCAATCTTTCTGAGACTTAGCGCCACGGAGGCAGCATGCCTCTC
ACTCCAGTGCTCTGCCTCTTTGTAGCAATGCAATCGTCCCTTCACCACAAAGCACCTCCTCCCCTAGCCCATGATGGCC
ATTAGCTTCTCAGGAACCAAACTGGCACTGGCCTAATTACAGTCACTCCAGTCCTTACTCCCCTGTTCACCTTAAATAA
TTCAATTTTTACAACTTGAAGGCAAATTTTTCTAGAAATTATCTACTTTAAATATGCTCATTTAATTCCCCAAGTCCTT
TAAAATGAGTATTTTATCTCTGAAACTGAAAACTCTATGGTTAAAATATTTTACTTCTTATGCCTGCCAAACTTAGCCA
TTAGCCCTCACGTGCTACAGCATACACCTCTAAATTATTCTACAAATGCTTAGTACTATATTATTTCACAAACATAGCA
AATTTGCCAGATAAGTTATTATTCAAAAATATCATCTTTGGGAGCTGAAGAGATGGCTCAGCAGTGAAGAGCACTGGTT
TCTCCTCCAGAGGACCCAGCTTCAATTCCCAGCACCCACATGGCAGCTCATAACTGTCTATAATTCCAGTTCCAGGGTT
TCTGATACCCTCACACAGATATACATGGAGGCAAAACACCAATGCACATGAAACAAAAATAAAATAAAATAGAGATATC
ATCTTTGTAGTATAACAAAAGCCCTGCCCTTCCTGGTTTAGGTGCTGTTCAAACTAAACACCACACTGTTTAGAACAGT
CTTTTTGTCCACTAGATTTTTCAAAGAAAAAAACTGAAGCCATTCTTACTGAATACTGTTATTTATTCATTCTAATTTT
ATTTCCAGAAATTCTAGAATTTTTATTTATCTATTTACTACTAGTATATGGTTACAATGTGCTAAACACTGAGACTAGC
AGAGGAGTAAGATCAGAGATCACATTGTCCTAGCCATGCATGGGAAAAGCAGGACCTGAGGGGCTAAGGTGGTCAGTGC
AGTGCTGTGCAGTGCTGTGCTGGAGTCAAACCAGAGGCGCAACTTCTGAGCCTGACAAGAAGTCCATGAAGATTATCAG
AAAACCAACTGGAGGTCGGGGGAGGGGGAGATGACAACTATGATTAAAAACAAATTTTTTTGTTCCAATATCAAAAGAC
TAATAATCTTTATTTAGCACTCATGGTGGGTGGAGTACACCTTTAATCCCAGCATTTGGGAGGCAAAGGCAGGCCAATC
TCTAAGTTCGAGACCACCATGGTCTACATAAAGCAGTTCTAGGACAGCTAAGGCTACACAGAAAAAAAACTGTCTCAAA
AAAACAAATGATATCAATACTAGTAATAGTAGTAATAATAGTAAGTTTTGTTTGGCTCAGTCTAAATAAAGCACATATA
CACACACACCTCAGTAGAGATCACAAGTGCCCCACACACTCTAAAAGGCCTACAGCAGAGGGCTGCACGAAACACAGCA
CACTGAGGAGCAATGTATCAAGACAAGCTAGAAAGTGTAAAGTGGCACTTCTTCTTGTTATGTTTGCCTAAAGGCAAAG
AGCTTATGTAAATATTTTATCTTTTACCCACACCAAAAAAATTAGTATACAAGCAATTATAATGAGTTTAAAACAAAAT
ATCAAACTCTTCCCAATAAAAAAGTTTACAAATGTGACAAGCAAAATAAAGTTTGTAACTATGGCAAAAAATAAAATAA
AACAAAAAGCACTACCTATCACACTATGCCATGCTATGCATGCTATGCTAGAATATACTATGCTACCAAATACTATAGA
TTTGCCATTAGCTAGCATTCAATTCAAAGCCAAGAAAATCTAAATCACACCATAAACAAGAATGAAGGAAGAAAGACAT
GCCCACTGTATCTTCTCCAAGAACAGTTTCAGTGAGGGAATACAGAAGAAAGCTGAACTGAATGGGGCCTAAAAGGCAA
AGGGTGAAGTGGGAATTATCAGAATACACAAGTCTTCAAAAATTTGATTCTAGGGCTGGTGAGATGGCTCAGTGGTTAA
GAGCACGGTCTGCTCTTCCAAAGGTCAGCAGTACGATTCCCAGCAACCACATGGTGGCTCACAACCACCTCTAATGGGA
TCCGATTCAATGCACACACTTCTGGTGTGTGTCTGAAGACAGCTACAGTGTATTCATATAAAAAAAGATAAATAAATAA
TTTTTTTAAAAAAAGATTTTGACTCTGAAGAACAGAGAAAGAGGAAATATTCTGATGGTTTAAAGGGGTTTAATTGTTT
TCAAAATGGAAAAAGCTCCCAGAATACTTTCTGTCCATATAAACCATTCTGAAGAAAGCCCTAAAGAGGACGTCACAGC
AGGAGCAAGTCAGCCTCAGACACGTCCACAGAGCACAGGCTCAGATGAAGGCCAACTGGCAGAAGGTTAGGACAGTGTC
TTCTGTATTTTCAATGAACTAGGGAGTGAGGCCATTTGTGAATGCTAGAGAAAAAACAACTTCATTTCACCAATGCACT
GTTTTACTCAATCAATGTGAGAATCAAAGTTCATCCTAACAACTTCAAATTTTCAATGTGTAGCTAATTATCTACCTGC
CTTCAAGACGCGTCACCTCTGCCTGCAGGAGTTCTTCACTGCTGTGGCTGAAGTCCAGCTGGGAGAGCACATTATCCAA
GACTCCTTGCCCTGGAGAGCTGTACTTCCTTTCCTTCTGACAGTCCTCCAGAGGCCTAAGAACAGAAGGAGGGGGAAAC
AGGTCTCTCTCTTATCCTGGCAAACAAACAATGCCAGCCAGGCTGTTGTCCCAACTATTCTTTTAACTAAGCTCCTTAG
AACTGGTCAGTTTGTAGCCTGCTTTGGGGAGGAGGGGGAATACCTGGAGAACAATCCAGGGTCAGCAGCAATTTAATCT
TTACTAGGGTTTGGGGTGTGTTTCAAGGGGAAAAGCCGCATAAAACAGCAAAACCATTTTGTTAACACTTGAATCCTTC
TATCAGAAAAACTTTACAAGACAGTCTGGCAAGAACAGAAGCATGCCTGACTTACACAAAAGGAAGCTCATTTACTCTA
TTATTTTTTACTATAACCCTGCCCTAGCAATAGCCCAGGGCGGGAGATTTATAGCTTAGTTGGTAGAGAATTCCCAGGG
CATACAGGAAGCCCTGAGACTGGGTGGGGTCACAACACTGATAATCCCAGCACTTAGGAGGCAAAGGTAGGAGGATCAG
GAGGAGTTCAAGGTCATCCTCAACTGCACAGTAATTTTGAAGGTAGCCTGGGGTAATGCATAAAATCCTATTTCAAAAC
ATAGTAGTAGTAGTAATAATAATACAATAATAATGATAACAATAAAGTCAGTTCCCAAAGGGCTAGCTAAGTGACAGGC
TGTTACTGATGGCAAGCAACTACTTTCCACCATACCTTAGTCTTCTAAGGCAGAGAGGCCCTCCAAGTCTAACAAAGAG
AGATAAGGAGTTTCCAACCTTATGATCCTCAGAATCTTAGAGACTTAAATGAAAAGGAGACAAAACCATCCATACAAAT
TTTAAGACAATAAAAATCTAACTCATAAATTAAAGTCAAATAAGACAAGATGATCAAACACAGAAATGAAAATATTCGA
TATTAAAATCAGCATTCAATGCCAAACTGTCAAACAGGCCAGCAGACTAACATTTACGGTTCAAACAGACCTTTCTACG
GACTGCTGAGTGCCAACTGCCTTTAATGTCGTTTGCAGTTTCTCCTGCATTTTTGCCTCAAGGATGAAGGTTTCTTGAG
ATTGAAGACTTGCCTTCAACTCTTTCTGTTCTTCCTGCAGAGCCTGCAAAACACTCAAGAAAAAAGACAGATATTATTA
CAACTTTCCTTTAGCTCTCTCTTTTTCCTGCAGAGACTGCAAAATACTCCAGAAAAAAAGTCAAATAATATTTCAATGC
TTCTACTTCATGAGAAAAGCAACCAGGAAGGCTGACTGCAAGTTTTATGACAAGGACAGTATGAATACAGCTATCAGGA
GCTGAGCCACCAGCCTCAGCCCAAGAAGGAAGCTGGGCATCCAGGACCTAGTCCTAGGGCATGCATATACCTCCCTAAG
TTACTCATTCTAACACACACCTCTAGTAACTTCTCAGATTCCCTTAGTTTCTCCTCCTTCTGCCGATGGTCCTGCAGGA
TAGTCATATTGGTTCCCATCAGGTCGTTGACCCTTATGTTAAGGCGCTCTATTTCCAGCTCATTGGCACAGATGGTGTC
CTTAGCCCGCTCAAGCTTACTGTTCAGGTGTTGAATTTCTGACTGGCTAGATAGTTCCACGGACTCTAATTTCTGTTTC
CGGTTGGCAAGCTGAGCCTAAAAACATATAGGGAAAAGAGAGTTGATAGAAGAATAATCAATAATTGTATTCACTGAGG
AAAGAAAAGATTCACTCCTAGGAAGGCTGGGCTATCAGCAGGTTTTGGACATTTTCTTTTAGTGGCATATCCTACTGAA
GTCTATTCAAATAAAGCCACTTCAGCAACTGGCTCAGGACCCGTGGAGTACAGTGTCTAACCTTTTGGCTCTGCATGCC
AGCTGAAGTGTTTTTGGCTTCTCACATTCTGTTTTTTTCAAGTTTTAATTCAGTATCCAGACTGGTTTTTTATCTATAC
ATTAAAAATGGACATAAATCATTTGTAAAGTTAAACTTCCCTTATTTTAAACATTTCCTAAAGGAAAGGCACCCACTTG
TATTTTAGAAATTCAAGCAGAAATATCTCTGTGGAATCCATCTACTACAGATTTAAAGATTCTGCTGTACATAACTGAG
GACCAAACTCTTTTGCCTTACAATATGGTGACACAAAGATGAAAAAAAAAAAAAAAAAAAAAAAAACGGAAACAAAAAG
GGAGAGGCAAAGATACAGACAAATAATTTTGTGTAAATTAGAGCTCAAGGCCTCAGAGTAGAGGCAATTTGTTTTCCAC
CAGATCCCTAGAATGGCACGTGAAAGAAGATTCAGGACAGGGTCAACGCTAACCACTATGCATGCAATTGGCAGAAGAC
TTTCAAAAACAAGAGGAAATCTTATACAGAATCCTCTGCACAATAAGTAAATGTAGAGATTGGTTGAAGGTAGGAGGAG
AAAATGGCTGTGCTCCGTTGCTGAGCCTCAGCTCACCCCATCCCTCACCTGCCCACCAGCCCTGGAGCTCACAGAGCAC
AGCTGGGAAAAGCTGTCCAAATGAGTGGCCAGGAGGCACATAGCCCAAAACCTCAGCACTGTGACACTGTATAACACTG
TGGTCTGCATTCAATGCTGGAGCTTTAGCTGTGTCAGCAACTCTGTAACCAGGTAGTCTCCATTTGTTACTTTAGAATC
AGTCTCGGTCCTCTTTTATTCTACTATTTCACTCATGCAAAAACACAGTATCTTCATTAACTAGAAATGATAGCCCACC
ATTGCAGACACTAGCAAGAGTTTATCGAAAGGACCCAGATGTAGCTGTCTTTTGTGAGACTAGGCCGGGGCCTAGCAAA
CACAGAAGTGGATGCTCACAGTCAGCTATTGGATGGATCACAGGGCCCCCAATGGAGGAACTAGAGAAAGTACCCAAGG
AGCTAAAGGGAACTGCAACCCTATAGGTGGAACAACAATATGAACTAACCAGTACCCCGGAGCTTGTGTCTCTAGCTGC
ATATGTATCAAAAGATGGCCTAGTCGGCCATCACTGGAAAGAGAGACCCATTGGTTGTGAGCCTAGCCTTTTAACGGCT
GAGCCATCTCTCCAGCCCGAGAGACCCATTGGACTTGCAAACTTTATATGCCCCAGTACAGGGGAAGGCCAGGGCCAAA
AAGGGGGAGTGGGTGGGTAGGGGAGTTGGGGGGGGGGTGGTATGGGGGACTTTTGGGATAGCATTGGAAATGTAAATGA
GGAAAATACCTAATAAAAAAATTAAAAAAAAAAAAAAGAAATGATAGCCCAAGTTCTACTGTCTACTATGGCAATAAGA
GACTGAGCACTGAGCTGTGGCTAGTCTGACTTGAACAATGTTCTAACTGTGTAATACACAGTGAGGTGTCAATGACTTA
GTTTTTAAAAGAATGTAAATTTTGAACTGATAATATTTTGATATACTGGGTTAAATAAAATATATTATTAAATTAAATA
TATTGATTTATATATTTATATAATATATACTAATTTATAATATATTAATTTTTATATATTAATATATTAAATTAATCTT
ATATTTCTTTTCACCTTTTTAATCTAACTGCACAAAATAGTAAACTGTGTAATTCACATATTGCTACAGTGGTACTAAT
TTCAGAAATACCTCTTTTAATCCTAAGCAAGTCAGAAAATCACAGAAACAACTCCTAAAATTCCTCCTCTTAACTGGCC
AGCTTTAGAAGCACTTAAAGGTAAATATATTTGAAATATATATTTTCATGCACAAATACATTTTCTTTCAAGAAAGCAG
AAATGCCCTTCTTCACTAAGCATTCAGAAGGGTGGAGTTATCTCACTACACAAAGGGCAATGAAAGTAAGGAAACCATT
AAGGCAATTTGCCTTAGGGAGACAGAAGGCACTTCTGTCAAGGCGCACCAGGTGACAGACACCGCAGAGGCATGGTGCA
CACTGAACTCAGAGCCTTGTGGGATCCTCAGGCTGCTGCCTACAGGGATGCTATGGCCTGCGTGGCCGCATCTGAAGGC
GACCCAGATGGGCAGACAGGTACTCGTGCTGCAGTGGAAAATCAAAGCCTTTGCTGCCTCCAACCCCACTGTTCTTAAG
CAATCTATTTGTGACAATGGTTTCCTTGAAGCTCCAAAACTACATTAAGAAAAGAAAAAAGAAAAAGAAAAGCCAGACA
AAGGATGTTCTCGGGTTTTTTTGTTTTTTTTTTTTTCCCTAAAACAAGCATGGAGTATAAAACTCTAATAGCTGCATAG
ATGAATCCAAATACATGACTCAAAGTGCGTGAAGCTGTTTTTGAATCTTCTTCAATGTTAAAAATATGAAAGAGTTTTT
TTCAGTTTTTTCTCATAACATTTACCCAGACTCTCGGATTTTCTGCTGTCCCACATATCAATTAAATGCAATTAGTCAA
GCTTAACTGGATTCTGCCTCAAACAGCCACTGTGAAAGAGTAGTTTTTAGACAACTGGATAAAACTGCAGACTGAATAC
TAAGTTATATTACTATTATTTATCCTATTGATGTTACCCTATCATATTATAGGGATTTTAAGTCCTTGCCTGTTCAAAA
ATGAGTTGATGCCATGATTTGCTTTGTAACATTTTAATAGCAAAAAATAAAGAGTAAAAGTGTACCCAGTTTTAGCTTT
CCAAGATGAAAGAAGTGGGCTGGATAAGAACGCCATAGGCTACTCTTCCAGAGTTCAATTCCCAGCATTCACCCTGTAC
CTCACACTGATCTATGCCTCCAGTCCAGAATCAATGCCCTGTTCGGGCACTGTGGCACTGTGTAACAGTGGTCACATAT
ATACATCAAGGAAAAATACCAATACAAAGAAAATCAATCTTTTATTTTTTTAATGCCATGTCCCTCTTTAAAACAAAAA
AGGAAGAAGTCTCAAAGAACAGCTGCACAAAACAATGTGCACATGTTGTCAACACTACTGCGCTCAGAAAGCCAGTCAG
GGTGGCACAAGATTTAGCTCACTGGTACAGTATTCGCCAAGAATAAGTGAAGCCATGGGTTCAATACCACACGAAAAGA
AGATGAGGTATAATTTCTGTTATGAATTTCTAATGCTCATTTACATACAAGATTGGCAAGATGATGACAATCATTAACA
CTAAGTAATGGGCACACAGAGATTTAATAAGTGTTTAGTAAAGATAACAACACAGCATTGATGGACACAAACTGAGTTA
AAGGAAGCTTTCATTCTTGGTTAGTTTGCCAAGGAAAAAGAGAAAGACATTTATACCTAATACTTTTAACTAATCAGAT
AACTTTATGTTAAATAAAACTATGAGAAGGATTATTCAAATACCCTTAGTGGGCACAACAGATGTCAGTTCTTTCAAGG
TATCTTATTCTATCTCTAGTTGACTGCCTGGTAAATAACAACTCCACTTTTACTAACAGTGAAGTTTGCATTATATGGA
ATTAACCACAATAGTAAATTGATAGCAGTGACTTAAAGATAATAGTCAATAAACATGAATAAAATGAGGATTGAATAAA
CAAAAACTCACCGGGCAGTGGTGGTGCACGCCTTTAATCCCAGCACTTGGGAGGCAGAGGCAGGCGGATTTCTGAGTTG
GAGGCCAGCCTGGTCTACAAAGTGAGTTCCAGGACAGTCAGGGCTACACAGAGAAACCCTGTCTCGAAAAACCAAAAGG
AAAGAAAGAAAGAAAGAGAGAGAGAGAGAGAGAAAGAAAAGAGAGAAAGAAAGAGAGAAAAAAGAAAGAAAAAACTGAA
GTGTTTTAGAGTGTTTTAGATGCTGAAGAGGCTAAGCCACAAGTAGAGGAAGGCAGGCTTACCACACAAAGACTACAGA
GAGAATCCAAGTAAGACAGAAAGAGCATGGCAGTAAGTCCGATTCGGTCAATAGCCATGCATCGGACAGCTTAAGCCTA
CCTGGATTATCTCCGACTGTTCAGCCAGAGCCTTCCTCTGTGCTTCCAGAGAAGATACCTGTTGCTGATAAATCAGACG
CTGCTTCTCCCAGTCCAGAGACTTCTGTCGGAATTCCTGGGGAGGCAAGTGAAGTACTTAACATCTAAGTGCTCCCAGG
CTGTGAGGCTTGCACAGCGCCCTGCTCCCTGGGCGCCCTTCTCTCAGCAGGAGCCCCATTACTCACGCTGCAAAGGAGG
ACTGGATCCTGCTCACACTGTGCCTCTACATGTCTGTCTTTCTAAATGTAACACTTAGTGGTGAAGAGGAAGAAACGGC
CATGACAGTGGGCTCAAGAGCATTATTGCAGATCTGCTGCCACCTATGGGCTGTTACCATTTTGTATTTTTAGAAAAGT
GACGCTAAAATAATAATATTCCCTTTGCCATCTAAATTTCTAACATTCAGACCCAGAAACTGAATAGATTGGAACAGAT
TTCTAAACCAGTAACTCAGCAGCCTGATTCTCACTTCCTATCCTGCCAATAATAGTATCTTTGATAGTCAAAATTTTTT
ATTTCGTAGAATTGCAAAAGCTTGAATAATATGGAAAATATGCATCACATTTTATCAGGTACTTATAATCCAGAGTCAA
ATAATTCAAAAATAATAGGTAATCAACAGCAATTTCAAACTCTACGTCATGAAAATGGGCCCAACTTTTCTGAAAATAA
AGCAACTCAAAGGGAAAGATCCTCAGCCTCCCCATCTTTGTCCGCCAGGGATCTCGAGCTCCAGAGCTCCTTAGCTAAG
TGAGAAGTCAGCTTTCTGATAGTCCCTTCACTCACAGTCTGTCTGTCTATATGACATGTGCCAGCACTGACTCAGGAAT
AGCATTTAATAAGAATAAACTGAAACCCAGCCCCATTCAAGTGGAGGTGATAGACATGACATGCACACTGAATGAATGA
ATGAATGAATGAATGAATGAATGAGTAAGAACGTTCAATGAAATCAAACAACATAAACAGACTTTTGAAGGAATTCAAC
AAGGAAGCCAACAAGCATCTGCATGAAGTAAGAAGTCAATACAGGCTATGAATGAGGCATTCAATAGAGACAGAAATAC
TGAAAAAAATCAAATGGAAATTTTGTAAATAAAAATCTCATTGAGTCAACTAAAAGCTCAATGGAAAGCCTCTCCAAAT
AGAAAGCAGAGTTTCAGGGCCTAAAGACAAGGTATATGAATTAGAACATGAAGACAATGACAAAGGTAATAAGAAAATA
TGAAGGGAAATGACAAGATCCACAGGACATTGTTAAAAGGCCCAATCTAAGAGCTACTAAGATGACACGGTATGTGAAC
AATGCTTGGCATGTAAGCATATAGGCCAGCTCACTAGAGAATTCTAGGCATGGCAGCAGACATCTGAAATTCCAGTGCT
GAGGAGGTAGTGACAGAAGAATCAGGGGCTGGAGAGATGGCTCAGTGGTTAATAACACTGACTGCTCTTCCAGATGTGC
TAGAAGAGCAGTCAGTGTTATTCCACATCTGTAATGGAGTTGGATGCCCTCATCTGGTATGTCTGAAGAGAGTAACAGT
GTACTCACATATATAAAATAAATAAATAAATCTTAACAAAACAAACAAACAAAGGATCAATCTATTTGTTACTTGCAAG
AAACATACCTTACAGGTAAAGACAAATATTGCCTTAGAGGGAAAGAACAGATCATCCACTATGACCAAACACGTAACAT
TACAATGATACAAGGATGGTTCAACATACATATTAATGTTATCAAATTATGATATATACTATATATTAGCAAATATCAT
TTATCATATTAATAGAACAGAAATCACATGATCCTCTTAGTTAATGCGGAAAAGACTTCTGATATAATCCAATAGCCTT
GAAAAAACTAACAATAAAGTTAGTTCATATCTCCACCCATCACCAGCTACACACTAGAGTAAGCTGGACCTACCCCTCA
CAGGACAATGCTGGAGAGCTGGCCCTGGTGGCACGGGTGTAGATTCCAATGGCATGAGTGCAGGACAGCCAATCCCACC
CCTTGCCAGCTGCCAAATCACCTGAGTAAAGTAGGAGCGCTGGCCCTGGTGGTGCGGGCACAGGAAAGCTGGCAGGCTG
ACCATATACCAGGCCCAGATCCAAGGCTCTGAGTTGGCCAACCCCAACATCTATCCCATCTGTGAACTGCTGTAACCCA
TGAAGGGGCCTGGTCCTACAGATTCAAGGCTGGACATCTCCATGACACAGAGTAACAACAGGATATCTGAGACAAGTCC
AATTGACAATCCAGTATTGATAGTGTAGCATAAGCCAGGGGGCTCAAACTGGACCAATGACTCACTCCAATGAACATTT
GCAAGTGTAGCTGTTTGGACAAAAGGGGATACTATATGGCACACTGCCTCTCCCACAGGGAGAGGTTTGGGGTTTTTTG
TTTTGTTGGTTTTATTTTTGTTTCCTCTTGGCACTGGGGGGGGGGAGGAAGGGATGGGGAGATGAGTGGGATTGGGGTG
CAGATGTGAAATTCACAAAGAATCAATTTTAAAAAGGCTATATGTCAGCCAGGTGGCAGTGGTGCTCATCTTTAATCCC
AGCACTTAGGAGGCAGAGGCGAGCAGATCTCTGAGTTCAAGGCCAGCCTGGTCTACAGAGTACCTTCCAGGATAGGCAG
GACTATACAGAGAAACCCTGTCTCAAAAAGCCAAACAAACACAAAGAAGTACAGCTCAACATAATTAAGGACATATATG
GCAAGCCTATAATCAACACACTAAATGGGGAGAAGCCCAGAGCACTGCCACTAAAAGGAGGAATGACAGACAAAGGCAA
ACACTTTTTCCACTGTTACTCAAGTTTGGAGTCTTAGAACAGAAAGCCAAAAAGGAAAGAAAAGGGATACAGCTAAAGA
AGCTAAATGATCCTTTCTTTACACAAAATGGTTCATACATGAGACTCTGAATACCATCACTCAACAGCTTCTAGAACTG
ATAAACACCTTCGGAAAAGGAGCAAGACATAAAACCCACATCAAAACTCAGTAGCTTTTCCATAACCAATAACAGACAC
CCTGAGAAAGAACTCAGGAGAGCAATCTCATTCATAACAACTTTAAAATACTGCAGAATTAAATAAACACACACACACA
CACATATATTTATACACATACATACATATATACACAACACACACACACGTGTGAAATAACATAACTAGGGAAGTAATCT
CTTCAATGAAAACTTTAAAACATTAAAGAAAGAAATGAAAGAAGACACCAAGGACGGCAAGGCTTGTCATGCTCATGGG
TTGACTATCTTACTGAAAGCAATTTCATAAAGGTAAGAACTGAGGAATGTCAACAGGAGTAAGTGGATGACACAAGGAG
ATCAAAATAGCTACGAAGAAAGGACTCCAGGAAACTCCAAGGCCAGAGCGTGATCAGCACTATGTCTGTGGATGAAACT
CTCAAAGGTTATGATGACAAGGATTTTAAAGATAACAACGATCTGAATCCAGAGTCAGTGAAGTAACCTGGACCAGAGG
ATGGCTACCGAGGCTCTGTGCTCATGGTTGGTGGTGTAGGCTGGTGATAAGAATGTGTGGGAGCGCCGGGTGGTGGCGG
CGCACGCCTTTAATCCCAGCACTTGGGAGGCAGAGGCAGGCGGATTTCTGAGTTCGAGGCCAGCCTGGCCTACAAAGTG
AGTTCCAGGACAGCCAGGGCTACACAGAGAAACCCTGTCTTGAAAAAAAAAAAAAAAAAAAAAAGAATGTGCGGGAGCT
AGAAGAAATGGTAGCAGTAGAGAGGCTGGAGGAGAGGCAGTATCTGTGTGGTCCTCTGATTCTAAGTTCTAAATCTATA
AAAATATTGAAATATAAAAGGAATATTAAACATAGAAATTACAATTCCATAAAGTAAAGTATTGTGGTTGTTTGAATAG
GTTTGGCCCCCAGAGACTCATGTGTTTGAATGCTTGGTCCATGTAGAGTAGCACTATTGGGAGATAGGACCTTGTTGGA
GTAGGTGTGGCCTTGCTGGAAGAAGTGTGTCACTGTGGGGATAGGCTTTGAAGTCTCCTATGCTCAAGCTTCATCCAGT
GTAGAAGTCCAATGTCCTGCTTCCTGCAGATCAAGATGTAGAACTCGGCTCCTGCAGTACCATGTCTGCCTACATGCTG
CCATGCTTTCCACCATGATGATAATGGACTAAAGCTCTGAATGAATAAGCCAACCCCAATAAATGTTCTCCTTTAGAAG
AGTTGCCTTGGTCAGGGTGTCTCTTCACAGCAATGAAACCCTAAATAAGACATGTATATAAAGTACATTTCTATATGAC
AGATCACTCATACAACATTGTTAAGCAGTCACACAATGATATTGAACAGTTTCTTAATTAGGTAAGGTATAGATTCCTA
GGTCCCATGCCTACTACTGGATCAGACTCTGAAGTCCCAAAACATCCTGTGTGGGGCACTGGGCTATGCACAGACAGCC
TGGTTTCCAGTTGAAGCTAGATGTTGAACCCCGGGTCCTGGTGGTGATAATTCACCTACATGGGACAGAAGGAGTTCTC
TCATGTCTCCTGGGACCCTGGCTCCCACCCCCTCAGCCCCCCATAAGAGAAGCGTGGCTAGTAGTCATGTAGGCAATGT
CCCAAGCTTCTGACCTTCAGGCTAAACTCCTCCCCAGTTACCTAGCAACAGTAAAGATAGCAGCATACCATAAAAAGGG
CTGCTTAGCCCCTCCTCACTCTCTCACTCCTCTCTTGCTCTCTTGCTCTTACTCTTCTCTCTCTCTCCTCCTCCTCCTT
CTTCTTCTCTCTTTCTCTCTCTCCTCTCTCTCTCTCTCTCTCTCTCTCTCTCTCTCCTCTCTCTCTCTCTCTCTCTCTC
TCTCTCTCCTCTTGGCCATGGCCAGTCTCTCTCTCTCTTTTACCTCCTCTCTTCCCCCTGCCTTTCTATAATAAAGCTC
TAAATAAACTGTCTGTTCTTCAAGGCCCACTGTGCAGACTCTCGCCTGTGTGGGAACCTCTCCCCTAAGCCTTCTCTCC
CATAACCCCAGGGCTGCAGGGTGTTGCTCTGGGCCCCCTGGTTTCGGGGGCTGCCCCTTGTCCACCCCCCGCCCCCCCC
CGCCCCCGTCGAGTGGGGTCAGTGGCTTAGATGCCCACCTGGAAAGCGAGTGGAAAGCGAATGGAAAGCGTCTGGCAGC
CCTCCCACATCCGCCTGCCCAGAGCATAGGAGGAACTCTGGCCAGCGCAGGCTATCCTCCTTCCCCCTCTTTCCCCTCC
CCCTTTTAGTTCCCACAATCCTGAGTGACTGTTTCAATGTACTGGAGAAGAGAGGGGACTCACTGTCTCCTATCAGTAA
TCACAGAGTTATGGAACAAAAACAAAATCAGAGAGGTAGGCGAATCCATTGTAACTCATGCTCTACCTAAATGGGAACT
GAAAGGAAGTCACCTGATGAGCAGAGCACTCTCTACAAGAAAAGCCAAGCATCCCCCCGGTCACTAACACCTACAGGAT
CGGGGTGGAGGAGACAGAGATAAGGTAGGATGCTGCCACTAACTGATGCTCCGTGTTTCTCAGAATGGGAGGTACTGAG
TACTAGGATCTTCTCCAGGATACTGTCCCAGACATGTTGGCTATCCCTGCTACCTCTACCATGAATGCCAATTTCATGC
TCCATCATTTTAAAATAATAACAAAAAACAAACAAAACAAAACCTCCATATATTTCCAAGTGCTCTGTATGGGAGCTCT
ACACTCTTGAGATTAATTGATGTCTTGGAAGCCCATCAGCCACAGGGGAGCATGTGATGGGGACCGCCATCCTACACTG
ACTAGTTCAGTGGCAGCCCCTGAGATAGCAGGAACTTGCTCCAGAAAACAGCTCCATTAGAAATGGCATTTTCCTACAC
AATGGACAACTGCCGTTTCAACTGGGACTATTGCTGTAGACATACCTCTATTTTTCCAGTTAGCCTCTCAATCTCAGAC
CGATCCTCTCGAAAACTTTTGGTATTTCCTCTAAATTCTCTTAGTTGCTTTTTCTGCAGCTTTTCATAGCTTCTCTTCA
GTCTGCTTAACTGTAAAAACAGTTATTTAAGAGTTAGCAAACTGTAGTGGTCAGATGAATTGGGATTATACAGCAAGAG
CACTGCTAAATTACACTATTGAAAATAGATTGGCAGCATGTTAGAGCTGCAAAGCAAAATAAAAGAAGACATAAAAACG
ACAAGGCTGGGCTACAGCTCAGCTGGTAGAGCGCTGACCTGGAAAACGCTGGCCCTGGGCTGGACCAAGCTCCACACAG
ACTGGCTGTGGGAGTTGGTGCAGTGATCACAGAACTAAGTGAAGGCAGGAGAAACAGAAGGGCAAGGTCATCTTCAATC
ACATGAAGAACCTGAGGCCAGCCTGGGCTGCCTTGTCTTAAAGTGCATGTGCCTGCTTGCATGTGTATGTGAGAGAAAG
AAATGAAATTTATAAAAGTAATGAAATTTCCAGTCCAGAAAAGAAAAAGTAAAATTTAGTTTAATGGCAATTGTCATGC
ATTCATAGATGCCCCTCCACGACCCACTCCACAGAATAGAGGAAGCAGTGACTGTTCCCCTCTGAGCCAGTGGTTCTCA
AGACATACTCCTGAGGGCACTGATGATGCACGGTCAGTAGGAGACTTTGACTTAAGCCAAAACAAAAGTAATTTCCCCA
ATATTAAAAATTACATTGAATATGAAAATAGAATTTTCTTTTTAAAGATTTTATTTTTAATTTCTAGTTATGTGTGTTT
CTCTCTATGTAGGTATATGCATGCAAGTGCAGATAGATGCCTAAGAAGGCCAGAGAGGATGTCCGAGCCCTCTAGCTGG
AATTCTAGGTGTTCTGAGCTTCCTAACATGGTACTGGGAACTGACCCAGAGCCCTCTGCACGAACAGTACACTCTCAAC
TCTCACCTAGCTTTGCCTCCCAATTTTCTTGCTTAAAAAAGTGTTTTGAAAACATTTTTATTTTTTATTAGGGGAGAGA
GGAGAGGAGGTGTGTGTGCATCTCAGCCCAGCAAGAGGAGGTCAGAGGACAGTCTGCTGAAGCTGGGTCTCTCCTCCCA
CCATGTAGGTTCCTGGAATCAAATTCATGCATGAAGTTGGGTGGCAGGTGCCTCTACCCAATGAGCCATCTTACGACCC
CTGCTTTTTGCAGCTCTTTTTTTTCTCTCCTTTCCTTTCCCCTTTCCTTTCCTTCCCTTTCCTTTCTTCTTCTTCCTTC
CTTCCTTCCTTCCTTCCTTCCTTCCTTCCTTCCTTCCTTCCTTCCTTCCTTTTTGGTTTTTAGAGACAGGGTTTCTTGT
GTAGTCCTGGCAATTATAGAACTCACCAGGCAGGCTGGCCTCTAATTCACAGAGATCCATCTGCCTCTGCCTCTCAAGT
CCTGGGATTAAAGGCGTGCACCAGCACCTCATGGCTCCCAAAATATTTCTAAGTGCTTTGTATCTTCCAATATAAACTT
AGAAATTAAAATATTAAATGTTGCCAGGCAGTGGTGGCACATGCCTTTAATCCCAGCACTTGGGAGACAAAGGCAGGCG
GATTTCTGAGTTCAAGGCCAGTCTGGTCTACAGAGTGAGTTCCAAGACAGCCAGGGCTCTATAGAGAAATCCTGTCTTG
GAAAAAAAAATTAAATGTTCTGTTATCAAGATTTCAATATTATATAAACAATTCTTTTCCTCTCAGCTAACTGCTGGCC
TAGGTACTAGCAAACATGCTTGGACAAGATTCTTTACCTAGATTCAAGGTCAGAGAAAAATATTTCTACCACATTCTTC
CCACAGACTATCACACAGGCTGTGCAGTTCTCACCACTGAAATCAGAAACAAGTTGTCAACACAACAGTCAGGAGTTAC
AGAAAGAGAACGACTACACCTCACGGCACGGCACACAATGAAAGGCTCACTTCCTCCTGCAGCTTCAGTAACTCCTCCT
TGTACTCCATGGCCATTTCTTGCTTGGTTTTCTCATGTTCTTCTATCTGCTGATGCAATATTCCAACCTAAAAGCAGAA
CAGTCATTAAGTATATTGCTTAATTTCCAAGTACTTGAAAAGTTGTCTTTTATTGTGTTGAATCTTAGCTTAAATCTAC
TTTGCTATAAAAATATACAACTTAATCTGCTGGTTATGGTGATAGCCGCCTGACTGCAGTAAAATGTCCCAGTCACTTA
AGAAGAACACACTTAAGATTAACTAGGTCAAGTTGATTCTGATCAGCACTCTACCTTAAATGGCCTTTCCGTGCGCTTG
TTCTATCAACAGCAAGAGAGCTGTGTTACTTCTATAGGTTCCATGGACATAATTTCTCCCTATCTCCTGCCTGTGCTGC
TTGGTACACTTTAAGGCTGTATGGCTACTTACAGTTGGATTATTAGAGTTCCAGAGTTGAGGTCTGGGACTATTCAAAC
TATTTTCCATTTCTATTATGACTTCTCTGGGTCATTCATAACTTATTTAATTTCCAAATACACAGATTTTTTTCAAGGT
TATCTTTTTCTAACTTACTTCTGCTATGTTAAGAGAGTTTATCTATGATTTCAAAACTTTGTTCATGGACCAGGATAGA
GCCATTTTTGGAAATCATTCAGTTTGTGCTGCAGAAGAAATGCATGCTCTGCAGTTCTCTTGTGTTGAGTTCTATAGTT
ATAAACTGGGTCAAGTTTGCTGATCATGCTGTTCAAATCTGTATCTTTATGAATGCTCTCCATCATCAGTTACTGAGAA
AAATGTGTTAAAACTCTCAAGCATGACTCTGGCTTTATTATTTCCTACTGACAACCCTGGTAATTTTTACAGATTTATA
AGGAAGGTTAAAACTGTCTTACTATATTATTTGGTCTTATTTTGAGGTTTCCTAGAAGTGCAATCTGAGAGTAGACTTC
TGGGTGGATGAGGAGAGTGACCTTGGAAAGCATAGTGAGGAGATGGAAGGAAGAGCCATAACGGCCCATTCCTGGGCAG
ACTACGGCAGTAAGCAGCCGGAGCTGTCACTGGGAACCTACTAAGGAAGCAGACAGCTCTGTTTCTGTCTCTGTGAGCA
ACAGGAAGCTGCTGACTTCGCCCATCCTCACCCTGCTACGTCTAGACTTGGTGTGAACAGTTCACAAGGTCCCGAGAGA
AGCAAGCGCAGTGGAACAGTAAAGACCCAGAGAGCCACATGGGTGTTTGGCCTGTGTGTATCTGTGTACCACATCGAAT
GCCTGGTGCCATGGAGGCCAGAGGAAGGTGTGTTCTGAGACTGGGACTACAGACAGCTGTTAGCCACCACGTGGGTGTT
AAGAATGGAGCCTGGATCCTCTGAAAGAACAGCCAGTACTCTTACCCACTGACCTGTTTCTCTAGCCCACCACACTGTT
TCGTCTTTGCAGTCTAGCGTGGCTATCATTTGCTGGGCTTCCTTATCACCTTCCTGTCTCTGTGTAAGTTGTGTTCTTA
AAAGATGTGCCTAGGGGCTGGAGAGATGGCTCAATGGTTAAGAGCGCTGGCTGCCCTTCCTGAGGTCCTAAGTTCAATT
CCCAGTAACCACATGGTGGCTCACAATGCCCTCTTATGGTGTGTCTAAAAACAGCAACAGTGTACTCATATACATAAAA
TAAATACATAAATCTTTTTTTAAAATAAAACTTCAAAAAAAACTTTTAAAAGATTTGTCTAAACTTTAAACTCTAGATT
AGCAAATACCATTTACAGAAATCTAAACTAATACAGCTGGTATTACTAAAGTGTTATTTGACTCTAAGTGTAAATGGCC
AACCACTCACTGGGCGATAACTTTTTAACTCTTTGAAAATTTCACACATGTATACAGTATCATCACTAACCAAATCACA
GTTTGTGTTGTCAGTCCTGGTAGTATATTGGCCTTCAGAAGGGCAAGTGCCACGCAGGCAAGTCTGGCAGAACGAGCGT
GAGGCTTAGGGTTAGCATTTACCCACAGTTCGTATTCTTCCCAGACTAATCTGTATAGAAACTCAGAGACAATGCTTCC
ACCTACACTGCTTCCTGCTTCCCTTCCAGGATTCAGTGTTCTTCTTCTACTGCACCACTAAGTTATGAGGACTAAGTCA
GGAGCTAGTCTTAAACCTCCCCCCAGCACTACCAATATTAGTGGTGGTGAGACTAGAAATGATTTGATTTTCCTTCTCC
CTAATTTTAAAAAAGGTCTGTCTGAGTGGGTTTCCTTAACTTTGCTTACAAGGCTGGTGGTCCTGGCCGGGACTGTGTG
CATGCTAAGCACACTCCATCACTGAGCTGCTTCTCCTGCCTTGACCTGCTTAACTCTGACTGTTAAAAGCAAGGGCTCA
ACTTTTACATTTATTGGGAGGTAGAGGGGTAGAAGAATGCTGGAACTGAACCTAAGGCTTTCAGCATACTAAGCAAGCA
CTCTACTGCTACACCATGTCCCTATATCTTCAACTTTGCTGTCACCTAACTTTACAAGTGAAGCCAAAACCTATACTAT
AAACCTGGTGATGTTAATATGGACATGTAATATGATGAAAGTTATGCAAAGATTATGCAAGCCTTCTCCCATTTTCTTG
TGTGCTAGGGTTGCAGATGAATACCACCATGCTTGGCTGTATCACATGACCTCTGGGAATGCAAACCCAGGGTCTTAGA
GCTGCACAGTAAGCACTTAACCACCAAAGCCACGCCCTGGTCTCCACTGACACTCTCTTAAATACAGTGTGCATTATAT
GTTTCCAAAGTAAAATGCTCCCTGAAACAAATACTAATGTTCTTATACTTCCATAATAAAATGTCTTCTAAAAGAAACC
CTAAGAGGACTGGAGAAATGGTTCAGTGGTTAGAAGCACGGACTGCTCCTCTAGAGGACCCAGGTTGGATTCCCAGTAC
TCCACATGGCAGCTCACAACTGTCTCTAACTCCAGTTCTAGAGAATCTGTGACATCAGGCATATATATGGTGCACATAT
GTGTGCATGCAGGCAAAATACTCATTCACATAAAATAAATCTTAAAACAAGAAAAGAAAAGAAGAGAAAAGAAAAGAAG
AGAAAAGAAAAAAAGAAGAAAAGGTAACAATTAGAATAAGATTGGGCGGTTGTGGCGCACACCTTTAATCTCAGTACTC
AGGAGGCAGAGACAGGCAGATCTCTGAGTTCCAAGTTAGCCTGGTCTACAGAGCAAGTACCAGAACAGCCAGAGCTACA
GAGAAGCCCTGTCTTGAAGACACATACATACATACACACACACACACACCCCAAACAAACAAACAAACAAACCTAACTT
GGTAAACAAATGCTTACACTTAAGAAACACCTTTAGCATAAAAGATTAAACTACCACAAAGATGGATTAAAGGTTTGGA
CATGGGTTGCTCCAAGTATATTATATACATGCATGAACATGGGTGTATGGAACCATGTATGATTTAAAGCCAGAGTCAA
GATAACAAGAGAAGTGAAGCAGCCGCTCTACCTCTTTGTGCTTCATGTCCAGCTGACTCCTCAGAGCCTTCAGCTCCCG
ATCACGGATGTCCAAGCAAGTCTCCAGAGCGTGCGTCTGCCCCTCCCACTCCGACTTCTTATGAGCCACCATTATATCG
ATCTGTTTCATGAGCTCTTGCAGTTCTGCTTCACAGGATGTCAAAAATCCCCTACAAGCACCAACAGACCAGTCACAGC
TCATGCCCAAGTGGTTTCTCCTAGTTCACAGATAAAAATATCACAGACTAAACCTATCTGGAGAACTGAAACATTACAG
AAGCCAAGCCCAGGGTGCCTGGGCCTTTGCTGTCATATGCTATGCTGCTTAACTCCACACAATATTTTGCCCTCCTCTA
ATGAGTAACCTAAAACCAGGCTCACTTGCTGAAACACAGATGGATGCTTTACATCTGGCTTGCAAAGTGAGATTTCAGA
GCTACTAACCACATCTTCTGGGATATTTAAGGGATTCTTTTGGGAGAATCCAGGCAAGCCTGTGTTGCAGCTCTTTCAG
CCAAAATGAACAGTCTTAGTAGCAAACAGGCTATATTATGCAGGAAAAAAGATAACAAAGAAGAGCTGTTTCAAGAAAC
TCACCCTTGAGCAAGATCTATTGCTGTTATTTCTCCCAACTGTTATTTGGAACACACTGTAACCTACCTCACTGCTCAA
GCTACTGATACCCATCTGAGCTGAAGGGGAAAACTCAAGCTAGGATAGGAGCCACAGTCCTCCCTAGTAATGCTGAGAG
AAGGCAACTGATGTCGGAGGGATGCTGTCCACAAATGTGCCTTAGCTCAGCTTAGCTTTAGTCTAACAGAACCCAGGTA
GGTTTTTAAACTGCAATGAGCTCACAGTCAAAGGCAAACTTACCCACTATGCCCCCGATTTTGTATTCCTTCCAACAAA
GCCTCCATCTCTAAATCCTCTTTGTTTTGGCAACTAGGGGAAAAAAGAAAAAGAAAAGAGGAGTGAGAAAAATTACTGT
TCTGAATTCCAGGATTCCAAGTTAGGGTGAGTAAAAGCAATCACAGTGAGAAATACAAAGCAGCCTAAGAAGTCTGTAG
AAGCAAGGATTATCATTTAGCAATGCTAGCACCAAATACATTCCAAAAGCAAACAAAATCCTAACTGCTGCTGAAACTA
TCCTACAAAACCAATGAAACATCACATTACTCTATGCTTTTTTTTTTAATTGTTTAACTTAATTTTTTAGACACAATAT
GTCTCACTATGTAGCCCTGGCTGGTCTAGAACCAGTTATGCATGTAGACCAGGCTGGCTTTGAAGTCAGAGATGTCCAG
CTGCCTTCTGAGTGATAGAATTAAAGGTGTTTGCCACCAGGCCTGGCTTTCCTTCTATTGTTGTAGTCAAGACTTATTT
TTTAAGTATGGTCCATGGAAGCCAGAGGCAATGGAGCCACCAGATGTAGATCTAGGAACCAAATTCTGGTTCTCTTCAA
GAATAGTATATGCTCTTACTGTCTGAGCCCTCCTTCTAGCCCCTCTAATGCTTAGTTCTTAAAGAATGTCAGGGTGAAA
AGTTATTTTCTCTCAAGGGACATTAACACAAACCCTGCTACACATTGAGAGGAATCACCCAGGACCACTGCTGAAGGGA
AAGCAGCACTTCTCACTAACACAACAATTACTGCTGCTGACATGTCCCCCACTCTTCATGCCAAATTATATAGAACTGG
AAAATAAAGGTTTAGTATATTCCTCCTTCTTTCTAAGCTATTGGTTTCTCAGTGACAGAAAACCAATGTCACTCATCTC
TGAGGTCCAGAGCTGAGACCAGATTTTAACAGGGCACTGCTGCATGCCTTGGGAAACTTCATCATCCCTTAAGCAAACT
GCTCACAGATGCAGGACTGCCAAGAAGGATCTCGAAGGAAAATGAAAGGAGACAGAAAAGACTGCCTAAACAGAGAAAT
TCACACCATAAAGAAAGAATAACCCTTAAACTCATAAGTATCGAGAGTTGTATGATAAATTTCTCTTCTTCAGAAATCC
TCAATGTGTTCTTTCAGAGACCATCTTTCATTCTGAAGAAAGCGCCCCAGTAGGCTGGAAAGAAGGCCTGTTATTCTTG
CAGAGGACCCAGGTTTGATTCCCAGCACCCATATGACAGCTCATAACTCTCCATAAACTTAGTTTCAGGGGATCACAAG
TTACGTTTATAGTGCAGAGATAAACATGTAAGCAAAACTCTCATGTACATAAAATAAAATTTAAAAAGAAAAAAGAAAC
AGTCCTTTTTCCTCAGTTTTCAAAGTTTCAATTTAATTCTGAATTAAAGACAAGGCCAAAAGTTGCGATGTAGAGATTG
GGTCCCTCTGTCCTAATGCTTTTCCAGAAGTAATCTACTTCTCTGAGAAGTAGATTACCTTATTGAACAGTTTAGTGTT
GTACAAACAGCCCTCTACATAATAATAAAAGACATACTCCTTACCAAGAAATAGCCCAATGTGGGACTAAAAAAGAAAC
AAAATGGTCAACATCCCCTGGCAATGAATGCACAGACAGAAGACACACAGATGATTCTAGAGACACAGAAAGGCTGAGA
AGACCAGAGGGACAGCTAGTGTGTTGTAACTGCACCTATCTCTACATGTCCTAGCAAGGACTGCTGGCTCTTCTTGCAC
AGGGTGGCTAAGGCTCTTTCCTCCAACCAGTGTTCCTGATAGAGCTCACCATGACCCAGAGTAAAATCCTAACGGAGAG
GACTCCACGTTTTACTACACTTCCTTCTAAACCACAGTAAGGGTCAAATTACAGAGCAACCCAACCCAGCCTTTGCTCT
GCTCCAGAACTGGGAAGCAGTTGAGCCAACACTAGACAGACGCCCAGAAAACAATTCTGCTCTGCAACTAACCAGCTCT
GTGTCCTCGAACTTCGGCTTTTCCATCTGCAAAATGAAGGGTATGAATAGTAACTGCCAAGGTCTTTATTTCATCTTTT
TTATAATTATTTCTCAAAATAATTCCTACTAAAAATACACCTCTTTAGGTCATGCCTTTTTTGTTTTTTGTTTTTTGGT
TTTTTTTTTGAGATAGAGTTTCTTTGTGTAGCCCTGGCTGTCCTGGAACTCACTCTGTAGATCAGGCTGGCCTCGAACT
CAGAAACCCACCTGCCTCTCTGCCTCCCAAGTGCTGGGATTAAAGGCGTGCGCCACCACCGCCCAGCAGGTCATGCCAT
TCTTAAACAGTGATTATTTAATAATATATAATTTCTTGGCATTTATGTATGACACTATTTAATAACTAACAACGTGTAT
AATATATGCTAAACTTTAGTGAATGTTCACTTTATCTACCTGCCTCTTACACGTTCTATCTTCCTTGTAAAAGGCAGGT
GCTACCCCTTTGATGAAACAGGGAGAACGGCTTGATTATAAAAAGCTATAGGTTATATTCCTTAAGTTCAAACCCAGAT
AATGTGACCCTCCAGACCCAAGCAATGTGCCATCTTTTGGTTAACACTGCCTTCTTATGGTTAACAGGATCTCAGAGGG
CAACAGAATAAAGCTGAAGCATATTGCCTAATCACCACTAGGTGGCAGCAGCACTGTGATAAAACTACTCAGCTATAAG
AAAAGCTCAAAACTTAAATCCTAGTATTGAGACAAAGGAATTCTAGTTTAAAACAGGCACAGATGACCAGTAACATGCC
TTGAAAAACGTCGGCTAATGTGGAGTGTGTTTCTTTAACTTAAACACTCTACCCTTCTCTAGGATCCAATACACAGGGA
GGACTGAGATTTCCTGTAGATATTTATCACAGCCACATCAGTGACTCTGTCCCTAACCAACATGGCCTGCATTTACTCA
ATAAGTCTTGTTTCCTCTTTGATGGTGCCACGTGACTGTGGCTGAGAATTCTGTGTTCCTAATACTCAATACAGACTTA
AGCAAAAAGTGTTTGATGATCTGCCACCGCTTAGCACTAAGGTCAAATACTAACATAGGGCCAACAGGGCCCTCATTCA
CTGAGGGAATACTAGGAAGCTGTCAAAAGAGCATACATCTGTGGAGGGGCCTGTGATAAGGTGAAAACAGCTACAAAGC
AAGAAGAAGAAGTGTGTTCGCCTTTGGGTGCTTATTTACTGAAGAACAAACTGAGAACTACTGCCTTTACGGAGGGAAG
TGATCAGGTGGGGGAGTCAGGGATAAAGCTAACTTTCTACAGAGCAGGTTTGCTCTTTTACAGGGACGATGACAGTGCT
TTCTAACTACCACACAGAGAGGAAAGCAGCAGCACTGACCGGGATCCTAGCACTGGGGAGGTGAAGCAGGAAGATCTTA
AGTTCAAGGTCATCCTTGACTACAAAAAAACCCAGCTAAAGCAAAACTAGGAGGAAATGATACAATTTCAGGTATCAAA
AAAATGTTGGGGCTGGAGAGATGGCTCAGCGATTAAGAGTGCTGACTGCTCTTCTGAAGGTCGTGAGTTCAATTTCCAG
CAACCACATGGTGGCTCACAGCCATCTGTGATGAGATCTGATGCCCTCTTCTGTAGTGTCTGAAGACAGCTACAGTGTA
CTTACATATAATAAATAAATCTTTTTAAAAAATTGTTATATGTTCAAACAATGTAAACACATAGAAGCCTATTGGTTGG
TTGGTTGGTTGGTTTTTAAGATAAAGCCCTTGGGTTACTTTTGGAGGTTGCTACGTCACAGATTTAAACACATTCAAGT
CTATCTGTTTGTTTTAAGGTGGAGCCCTTGGGTTACCTTTGGAGGTTGCTAGGCCACCAATTCATTGCTGTAGTCTGAA
AAGAAAGGAACGGAGTTAAGCATTTTGCTTGCCATTCCTGTACAAACTGTACTTCAGGATAGTCAGCTGCGGAAGGCAA
GTTCATCATAAGAGTGCCAGCTAAGCAGCACTGGGCACATGACGCCACTAGAAAGCACCACGGTGGCTCTCACTCAGTA
ATCGGCCCAGGCACTATTAGCCATGAAGAGCTCCCTATTGACCAAAGGCTCTTGGGCAATTTCATGGTGGCTCAACTGA
GCAAACAGCACAGGAACCCCGACATACACAGCTTAATTCTAACATCCCTAGTGGGGTGACTCAGACATTACAAGTCTCC
CTGACAATGAACGCAACAGATAATTTTTATAATATAAAACCTATACCTTTGAGTTTTCTTGTACCTGCCATCAAAAATC
AAGGCTGAATCTAACACTGTATTTCAAAAGATCATTGTATTGCAACAGAACTTTCTGTAATTATGGAAACATCTTATCA
GTATTAATCAGACAATAGCCACTAGGCAGCCTGTGTGTGCTGGGCATTTCCAATGTGGCTAATGCATCTGGGGAACCAA
ACAGTTCATTTAAAATGTCAGGGCTGGCTGATGACTGCCACACTGGATAGTGTAGTTCAAGATTTAGCCAGTGAAGGAG
AGGTCATAGAAGAGGTAGAGGCATAAATTCAAAAATATATACAACCAGCTAAACCTGAGTGATAAAGAAACGGGAGGTA
AGAACCTGCTTGGGGGGGGGGGAGAAATAGGGGATTTTCAGAGGGGAAACTAGGAAAGGGGATAACATTTGAAATGTAA
ATAAAGAAAATATCATATATACACATATAATACATGTATATAATATATATGTATATATATGAAAGAAAGAAATTGAGGG
TTTTGATTATTGATTAGAGGGTTTTGAAGGCCTAGCAACCGAATGCAGCTGACAGTTCTCTGTAGGACTTTGGTCTACA
AGTCTACAACTTTGGTATTACAAGCACATATGGAATAGCTGAAGAACCTGAACTCACCGGACACTGACACTGTGTGCTT
TTTATAAATTTGTACTGATGTAAAAATAGTACTGTGGTTTTTGTAAAACTTAAAAAAGCAACTCTAGCCTTTGGCATGG
GGAGCACCTTAAGCTATGACAGTGTGGACTAATGCTGTCACCATCAGCCACCACACAGTACAGGAAAGCCTAAGTAACC
GGTCCCTACCGCTCCACGGGAGGAAGCAGCAGCAGCATGATTAACAATATCTCCATTGTTTCTGAAACTTCAGCATATA
CACATTTTAAAGGGACATGTGTCTGTGGTTTAAGTCTCTAACCCTCTTTAGTTTCGCAGCCTCCTTTACTATACTGACC
ACTGAGGACGACACAGTAAAAGACAGAGAGCACGGGACAAAAATTAACCTAAAAATGGCGCTGCATTCCAAATAAGATA
ATTCGTTTCATCATATCGTTAAGCTGCAGCCTTTAAGAATAAGTGTAAAAGCCGGGCGTGGTGGCGCACGCCTTTAATC
CCAGCACTCGGGAGGCAGAGGCAGGCGGATTTCTGAGTTCGAGGACAGCCTGGTCTACAGAGTGAGTTCCAGAACAGCC
AGGGCTACACAGAGAAACCCTGTCTCGAAAAACAAAACAAAAAAAAAAAGTGTAAAAAGCTAAAGATAACTATAGCCAT
TGATCTCTGACTTACATACAAAATATAAAATACAAATATTCGAGTTGTACAAAGAGCTATAATAAAGTAATACATTTTT
GAAACTCTAAATTGGCCTCAAGAAAGCCAGGCGAGGTGGCGCGGATCCTTAATTAGGAGACATAGGTAGGCCCATCTCT
GAGTTTGAGGCCAGCCTGGTCTACACTGCAAGTTCCAGATCATAGCTACAAAGGAGAGCCAGCGCTAGTTGGGGAGAAT
GGGGAGCACTTTTGGCAACAAGTCTACAGTCTGTAAATGAAAACTAGGTCTGACATCAGAATGCACTAACTCCACGATT
TTGTAAAGGACAAAAGCCCATTCACTGTCAAGTGTCTCCGCGTGAAAGCGCTGTTGTGGAATTCCCCTTTCCAAACTTT
CACTCCACCAATCTTGTTTCCTATGCTTGACTTGAAAGCGTGAAGCCATCTGCAATTCCCCTGCCATCAGGGGTTCCCG
CCACATTTCCGCACCTCTCATACTAACATCACCAAGGAACCCTGCGGGCTTCCCGCTGGGCGGCTAACGCTAAGGCCTG
GTCCTACACCGGCGGCAGCAGCAGCAGCCGGGTCCCTTCCCGGGAGCCATGCAGCCCACAGCCCGCAGCCCGCCCTCCC
GCAGGCCGAAGCACCTGGCCGGCCAGCCCAGAGTCCCATAGGGAGGAGGAGGAACCCAGGGGCGCGACGACCGGGCAGC
CGCAACCCGGCATTGGAAAGCCCCGCGGCAGGTGGGCCGCAACGAGCTGCGGCCGCGGGCCATGAGGCCTGGAGCCTAG
CGGCACCACCGTGGCCAGGGCCGAGACCGCACCACAGTTAGCCGCAGCTCGCCCGTCTTTGGGGCATGCGGTCGGTTAC
AGAGCGCGCCCCCGGCAGAGCGCGCCTCCGGCGGAGCGCGCCCCCGGGCCTGCCCGCCGTTCCTCCGGCCGGGGATGCG
CGCAAGTCACAACAAATCGCTGTCCGTCCCGCCCCCTGCCGCAGCGCTTGGACTCACCCACATCCGGGGCTTGAGAGCG
CCGCGAGTCACGTGACCCAGAAGAAGGGCGGGGCCGGGAGGAAGCCGACGCGCGCGCAGTGGGTAAGGGTTTTTCGCGC
CCGCGGCCCGGGTTTCCGAGGGTGGCCAAGCGTTCGCCCTCGAACTCCGGCGTCTACCGCTTCCAGAGGCGCGGGCCGG
TACTTTCAACCGAGCTTCAGAGCCACTTCTTTACTGCACGCTGTGCGGAGTGGAACGGAGCCCCCATGGCGACGCTCGA
CCTCCCCCCTTGCACTTCCGCATGTAGTATCCTCCCACCCCTCGAACCGGAAGTCGCTGCAGCGGGTGACTGTAGTGTC
CCAGGATGGTGGCCTCCGAGGGCCATTTTAGTGTAGGCGCCGTGCTCGTAGCTTAGAAATACATTTACAAAGCTGTGTG
CATGAAAGGTGTGTATGTCTCATAGGACGTACGCATCTAATGTCTAGAAAGAGATATCCGGGCAATGTGACAAGCTTTC
CTCCTGTTGTCACGTATTGTGCCCAGAGATCTAGGGGACTGGTGAAGGAATGGACCCTTGGGACTGCACTCAGCCTGGG
GTCTAAAGGAAACAATCTGAGAGCTGAGAGTTTGATATTATCTGGTATCCGGAGCGGGGGAACTTTCTGTTTACGTAAA
GGGTGAGCACCCGAGCACCTGACCCAGGAAAATAGATGTGTTTGGAAGGACGGCCAAGCATAAGTGATGAGGGACGGGG
GTGGGTGAAAAGCGTGCTAGTTTCTAAGCATGTAGAAGAGAGTCATGTTACAAAGTATTGAGGCGAACTTGTGGAAAGA
CTGGGTATCCTTACATTACCGCTGGAGACAGTAGTACCCTGCCAGTCAGTCACAAAGCTGACCTGACTTGTTTCATAGA
TCATTTGAGGCAAGCAGCAAAGCATTCAAGGACAGAGCCTGCTGAAAAGCTGGAGAGAAAGTCAGAGCACTTTAGAAAA
AGGTGCAATCCATAGGGACTGGGAAAGTCAGCACCAGAAGGTGTGAAATGCAGCCCAAGGCTCAGCTACAAAAACAAAA
GGCTTGTCAAACCCTGAGGGGTGATTAGAAGTGGCCCTTCAGCTGGCTTTGCGTTATCCACTAGCTAGGGTCTGGTTAT
GTCTGAGGAAAACTGCTAAGTTATCACCTGGTTTTAACGTGGCAGAGGGAGCTTCAGTGTGTGTGTGTGTGTATACGCG
TACACGAAAGTTGTGGTGGTGGGGGAGATTTCTGCGTACAGGTGGAGTTGTAGATGAGTGTAGAGGCCAGAGGTCAACA
TCAGTGTCTTCCTTTATTACTTTGAATCTTAAACCTGGAGCTTTCTGATTCAGTTAAACTGCCTGGCCTGGCAGCTTCC
CAGAGAACACAAGTGTGCCATCGTGCCAGGCTTTTTCTGCGGGTGCCAGGAATCTGAATTCAGGTCTTTTTGCTTGCAC
AGCAAGCACTTCACTCCACTGAACCTCCTCCCAGGCCCCAGGTTGTTTAATTTTTTAAAGTAAAAAGCCCTTTCCCTCC
TACAGACTAGGTAGATAGCGCTAGCTTGCTTGCCTTTATTCTGTTGGAGGCAAGCACATAGCCGCGTAGCACAGTTTGT
TTCTTGTATGAGTCTCCCTTCTCTCGGAAACAGAACAGTTCCTGAGAGAGCCACTGGTACTAATCCTCACAAGACCTTT
CCTTGGGTTGCCACTTTGTGGCAGGGAGCACAGTAGGGTGTGTTTGTGATAAGGAACAAACTTGGAAGCCAGAAACCCA
AAAGCACTGCTTGATGACTGAGTTAGACTGCAACAGTTTCAGGAGAAAAATGTGGAACAATCTGTGGTCTGGGAGCTTA
GCAAGCATCCTGAGCACTTGGACCAGGCACTGAAGAGAGCCAGCAAGATGGTTTAGTGGCTTAAAGGCGCTTGCTGCCA
AGCCTGGCAACCTGAGTACAATCTCTGGGACCCGCCTGGTGGAAGAAGAGAACCAAAATCCCCAAGTTGTCCTTTGACT
TCCACACGAACTTACACATATACATTGTTTCTTTCAAGAAGGAAGTAGGCTGGGTGTTGGGATCACAGCACAAAAGTTC
TTGCACCCTCACATATCCAGGGAGGCCGAGATGTTAAGCATGCAGGATTGTTCTTCCAATGTGAGAGATATAAAACTTG
TTCAGTCCACAAAGCTGGGAATTGGAAGTGATTGACAGCCTGGTGATCTGCGTTATCTGTTGGGCCAGGCCACATCAAC
CTACAACCAGGACCGAGGGTGGCTAGTAATTTAAATGACCCTGATAAGCAGGGGTTCCCTAAGCTCCAGAAGTAGCTAA
GAGACCAGATGATGTCTATGTTATTGTATGACCTTCCTTAGACCCTTAAGCTAGTACAGGTAACCTATCTCTAGAACCC
ATCGTCTTGGAAGAAATAACATACCCTAAGTCGAGTTTCAATGTAATTGCACCTCCCTGTGCACCAGAAACTGTTATTA
CACCAGGAAACTTTCTTCCAAATCATACTATGTTTAAGTTTGTTCGGAGTAAACCGCCTAGCATCAGACCCCCCAAGTC
TAAGCAAGACTGATAAAGTCAGTCTGAACCCAGTTTTCATTCTCACCTGACAGTTTGCTTCCCTCCCTGGACTCCTGCA
GGGCCCCCTCAGCTGGGGATGGAGGTCGGTTGGCAGTGTGCTTAACTGTAGTATACACAACACCCTGGTTCTGTAACCA
GAGCCGGTGGCACAGGCCTGCAATCCCATTGGTGGAGGCAGGAAGATTGGAAGCTTAAGGCTGCTTTTGGCTATAGAGA
GTTTGAGACTCTCAAAAAAACAAACAAAAAGGAGGACGCCCAGAGGGATAGAGAACCTGGAGCTGCCACCGTTGATCTT
GGATAGTTCTGTTGAGTTCAGTCTGGATGCTGTTTAAGAAAGGAAGTGGTTTCCTGGGTAAGCCCTGCTGGCCACGTTG
TTCCTGCCCTGAGAGTTAGCAGCTGGCCTATGGTTGGTCACTTCACACATCCCAGCTCAGTGTCACCCAGCTGTTCCCT
TAGCTCCTGTGCCTTTAGACTGCCAATGTTTGGGCCAAGACGGGTGGAGACTTGTGCCAAATCGTAGGGGAGGGCTTCA
AGAGCAGGAGCTGACACTCAGGTAAGTCGGTCTGAGTCAAGTCAGAGACTCGTCAAAGACATCACAAGCAGGGAAGGGT
GATTGTATAAGATGGTAAAGATAAAAGAAAGTTACATTTCCACGCAGTGTCTTCTAGGACCTTATTCTGAAGACCTGTG
TTGCTCTCTGGTTCCACTAGCAGCCTGGTGTGTTGATCAAGCATGTCAGCTGAGGTGAGGAGAGAACTGGAGTCTTTAT
GTTGTCATCTGACACATACAAGATGGGCCTTTCCTGGGGTAATGTTAAGCACAGCAACAAAACCAGTTTCAAGGTGGAA
GTATGAGGAATCCTAGTCCTGGATTATACTATACCTTCACTGTGTTAGAAAAAAAAAAAAAAAGACCTCTTTGGCTTAC
AGGCCTGACAAGATCAAAGCTGCAGGAGGATGGACAGTGAGGTACAGCGAGATGGAAGGATCTTGGACCTGATTGATGA
TGCTTGGCGGGAAGATAAGCTGCCATATGAGGATGTCGCCATTCCACTGGTAGGTTGTGATTCGAGCTCAGGAGAAAAT
GTGGTGAGACTTAGTGGAGGTTGGTGGGAAGTGAAATGCTAAGGCCCAGAAATCCTACTGCCTGGTGTAACTAGCTTCA
ATACAGGTCATACCAGGGCTGGAGTATACCCGGATTTTTGGAGCCACAAAAGCCCAGAGGGATGCCAAGTGTGAGTTTA
GCACATCACACCCAGGAGCAAGGAGCATTGCTGGCTTACCAGTCTTTGTGGGTTTTTTTTGTTTTTGTTGTTTTTATTT
GTGTGTGTGTGTATGTATATATTTATGTATGTATGTATGTATGTATGTATGTATAGTGTGAGAATGTCTGCCACCCATG
TGCAGATGGGGAGGGTGACAGAAGAAGGCATCAAATTCCCTGGAGCTGGACTTACAGGTAGTTAGAGATCACCCAACAC
AGGTGCTGGAACAAAGCTTGGATCCTCTAAAAGAAGAGTCGGTGATCTTACCACTGAGCCATCCCCCCAGCCCTTAGGA
TTTATTTCCATTTGTGTATGTGTGAGGGTCTGGGATACAGTGGAGGAGCGTGTGAGTACAGGTGCCCTCAGAGGCCAGA
AGAGGGTGTTGGGTCCCTTGGAGCTGGAATTAAAGGCAGTTGTGAACCACCCAACATGAGTGCTGAAAACCAAACAGTG
TCCTCTGCAAAAGCAGTAGAGCCCTTAACCACTGAGCCGTCTCTCAGCCCCTCTGATGCAGGCACACAGCCAGCTGCTT
ACAGGCAACCTTGCCTTTCTTCTTTGGCCTGTAGACATACAAGCTCTCAGTAAACCTTTTCTTTGCTGATCTCTAGGAA
ACTCCCTTAAAAGGCTGTTGTTGAACCTTCTGTAATAAGTAACCCCCCACTTTTGCTTATGTAGATTTAAAAAAAAAAA
CAAAAACTCATTGATGTTACAAAGGCCTAACAAAGAACAAGGCAAACAGAGTCTGGGGACTTTCTCTGTACAGTGTGAC
ACACGCTCTGGAAGAGCAGCTAACACAGCATGACTTCCAGGAGCATCACCTGCACTGCTGTTTTACAAAGAACAGACTC
CACTCGCTCACTCACCGGCACTCTCTTTCAAGAGTAAGAGGTGGGATGTGTTCAGTGGATATTCAAGTCGCAGCTTAGT
TCAAGTTCAGGACAATAAAATAATAAGAATCATAGCTACAATTTATTACCAGTTTTAAGCCATAGACTGAAATTAAGGC
TTGCATTATCTGTAATCCTTAAGACAGCCATACAGGGGAGGTGGTAGTGTCTCCGTTTGGCAGGAAAAGAGTAGGATTA
GAGAAGTACACTTACCTCAAGTCTAGTGACTAGTATGTCAGATTTCATAAGCAAAGCTCAAAGTCTGTAAGCTTTCATC
TGTCCACCCATCCATCCGTCCATCCATCCATCCAACAATCACTTCTCCAAAGTCTACTGTGTGCCCTAAGGGTATATGA
GCATAAGATAAAGTTCCTGTTTGGAGCCAGGGATCAAGAGAGGTAGGTGACTAACTAATGACACTACCAAGCAGGAGGG
AAGGATAAGCAAGTGGAGAATGGCTGGAGGCCTGGCATGCAGTAGAAATGCAGAGAAGATGTAATGCTTAAGAAAGAAT
CAGATGGTTCATGGTGCTAGACAGGGTACTGTAGCAAAGAAGAGTGTAGCAGGCCCAGGGTTTGAAACACGTTGGGAGA
TGGTGAAGCAGTGGGCAGCAATTACCTTAGTAATTAGGAGGCAGAAAGTTGGGCATAGTTTTTGAGAAAATGTTTGTGC
CCCACTTCGACCTCCTTCTCTAGGTATCTAGGGATGAAAGGTCTGTGGAGGGAAGCTGCCATAGCTCACATTTGTCTTT
CAGAAAGTGGGGTCTGTAACATTGACAGACAGCCTGATAGAAGAAAGGGCCAGGGGACATCATGTGACTGCAGTGCAGT
GATCTAGGCAAGGGAGGAAAACCACAACAGCACCAGGAAAAGTGCTGTGACTTGCTGAGCTCCGTACAGCAAGACCTAA
GGAACACTTACATGGTAGGGAATGAAGGGGGAGGTCCCTCTGCTGACTTCCTGGTGCCATGGTAACCTGATCCTGTGGT
CCATCCACACAGTGTTGTGATTCTAAAGGAATGTGTCTGTCAGTTAGCTTTTGCTGCATAACAAATTATCCTGAAACTT
AATGGCTTACAATAACAACTGTTTGGTCATAATTTTATTTCTTATTGGGGAGGTCTGGTCCACACCAGTTCAGCCAACT
AATCCTAGGCTTGCTGGTGCTGCAGTGTCAGGCAGCAGACTAGCTGGAGCCCCTCTGCCAGTTAACAGGATGTCTCCAG
GAAGTAGGGTGCCTGGCACTTGGCACACACCACCCTGCAGGTCCTCTGGGACCTCAGCCTCAGGTTCAGCAGCAACAGG
GAAGTGCAGAGCACTTAGGGCTCAGGCCCAGAACTGCCCCACAGCTGCATTCTGCTGAGATGGGCAAGTCTGTGGCCAA
TCCAAAGGCACAGTCAGGGTTAGACTCTGCGGCTTGGCAGGAACAGCTGCAGACAGCTGCAGCTGTTGGTGAATTTGTC
CCTGTGGATATTGTGCGTATGGTATTTTGTGTTGGGTGGGCATGTTTTGTCTAGTTTTACATGGTAGTATTCATCGCAA
GAATTAATAGTCACCAAGCAAACGATACATCATTTTAGGAAGCCTAGTATGCCATTAGAGAGCATGACATTAAGTCGTT
CTTAATCAAAGGCCTGTTTATTTTTTGTGACCTGTAGATAGTTATTAAGGTCAGATTTCTAAACGGACTAGAGAATGAC
AGTTCTCTGGCTCTGTTTACAGAATGTTGAATCTCACTTTACTTTCGGCATAGCAGACGCATGTTGTGCAAACACATTG
GGGTTTTCCTGTATGTGTGTACCTGTGTGCCATGACACACATGTAGAACTCAGCGTTGTCTGCAGTGCCTTTACCCACT
GAACCACTTTACTGGACCTGCTTCAAATACATCTGTACATATTGTAGCCAAGTATCATGTTCCATATGTTGAACATAAT
TTTAAAAATAAAAATACCTCCACATAGTATAAATATAAGTGAGAAATCCTAACTTTGAATATGAAAGCATCAGTTTTCT
AACACAAAGTGTAAGTTCAGGGAATTGCACAGAAATACATGTGGTGAACTCTGGAGCCATGGCTTATCTCTGTAAGAAA
ATGGCTCTTGGGGTGTGAGCTAGTCTTTACTGTGCTTTCCACTAGGGGGAGCTCTCACCTGTTACATTGTCAGTGTCCA
TTATCCAGCCCATATTGTCACTAAAGAAAGCATTTTGTTGCAGCATTTTTTCTTCTTGATACAATTCCTGTATTTGAGA
ACATATCAGCAAATCAGATTGGCCCTTAAAGCATTTTTCAATAGGTTTAGCATTGGTTCTTGTCAGTGTTCTCAGCCAC
TCAGCAGTGTGTGCCCTAGAGTACTGAGTACCCTCACAGCATGCACTGGATCTAATGTCCTTACTCTCCCCGTCCCCAA
GGCAGAGACATGGGGGTGAAAAAAAAAGCTCTTTTTTTTTTTTCTCCAATTTTTGAGAGTGGAAACAAAAGCCCCCAAG
GGTTTGAATTACCTCATCAACCCAATAAGCCAGCAAATGAAGGCTGTCTGGGCCTCCAGTCTCTGCCCTTAGCCCTCCT
GCCCCACTGCCTTTTATAAGAAAACCAGAGTCACAGGGCCAGCATTCCCATGAGGCTTCCAAGCAAGCCGCACTGAACA
GACCTCTGATATTTTCTATCCATCTGTTTGTTTAGGCTCTCTCTTTAGAGCATCATATCAGCTTTGCTGGCTGTTCCCA
GGCCCTCGGAGGCTTTCTTTCCTAATGCCTTTGTGTTTTCAGAAGTAACTGCAGGACCAGGTCCACACCTATAATCCCA
GCTCCTGGGAGGCAAAGGAGGATGGGAAAGAGACTTGGGGGTGATCGGCTGAGAGTGTGGCTCAGTGAGAGAGTGCTAG
TGTATCTCAGCAGGGCCCTGAGCGCAGTCCCCGGCCGCGCAGAAACACAGTTCAAGATTTGGGGCCAGCCTAGGCTACC
TGAGACCCATCTCAAGGATAGAAAAGACACTTCCTGATGGTCGTTACGGCTGTGCTTCCCTTCCAGAGTGAGCTTCCTG
AGCCCGAGCAAGACAACGGAGGCACCACAGAGTCTGTGAAAGAACAGGAGATGAAGTGGACAGACCTGGCCTTACAGGG
CCTCCACGAGAACGTCCCACCCGCTGGAAACTGATGCCGGGCTCCTTCCCACGGACGGAGTTTTCAAACATATGGGGAT
AAAAATGTTTCCTGTCGCCAAATGCAGATCTTTAGCAATAAATCAACACTCAAAAGCATATGCCCACTTGGTTTTCGTT
TTGTTTTGCTGTTTTGTTTAACCAACAAAGCACAGCATGAAAACATTCTGAGTGAGAAACTGAGACTTCCCAGGAATGT
AGACCGCGGCTCTGGAAGCCTGCGGCAGGTGATGCACACACTTGCGTTTGAACACCGTCGATTAAACGTTTGCCTTTGG
CTCCAGTTTTGCTTCTGTCAAAGTTTAATACTGCCCAGCTACTTTGGCACGAGTAGTTGAGAATACAAGCAGGAAAGGC
AGCAGAAACCAGGCATGAAGAGAGCCACAGACTCGCATTCCCATGCCTGAGACGTGTTCTGAGTGACGTTAGCCTGTAG
GTGCCTATGCCCCGAGCAGATACTCCACTCTCTGATAACTGGCATTCTTAAGATTCCCAAGCTACTTTTAGACATTGCA
TAAATGTACAGATTTAAAAGTTATCTCTGAAGAATTAGGGGATGGCTTAGTCAGGACCTAAGTTTGATCCCAACTCCAG
TAAAAATTGGGCTTCGTGCCATGGTAGTGTGTGCTTGTAATCCTGCTTAGCTGTTAGTCACCTCTGCTCTCCGTCTGTG
GTTATTGCCTTTGCACCACTGGAGAGGTTCTCTGCGTGAATGGAATACTCCTTTTATTCAGAATAATAATGGCATCTCA
TACCTCAGAGCCCCCTCAAATATAAAACACTGTGAAGCCAGTAGTCAGTACAATATTTATACAAAAATTTGTCAGCGGT
GGAAAGATGGCTCAAGTGGTTAAGAGCACTTCCTGCTCGTTCTTGGCACCCACATACCAGCTCATACCTGTCTGTGACT
CCAGTTCCAGGGGGTTCCTTACCCTCTTCTGGCCTCTGGGGGGGCTGCACACATGAGATGCATATGCATACATAATAGG
CAAGACACTCATAAAAATAAATTTTTAGAATTTCTAAAGACTCCACGGCATCAAGCCTTGGGGATAGAAGTACTGCTAT
AAAATGTCAGTTATTGGCTCTCCCTTCCAGCCGTCTCCCCGAGACAGTATAAGCACAGAACTCCAGAGGACGTGTATCT
TTGGCAGCCAGTATACAGAGAACGGGTGAAAAAAAAAAAACAGGGAAAGGGACATCCAAGAAGTACAGCATAGTAGATT
CCAGTAGATGTCATTCACTTTAAAGAAAGTGTGGCATTTCTTGTACCTGCCTTGGCTTTCTGAACAAACACCTGACATA
CAAGTGGCCAGGGAAAACAGTGAGGGGTGAATGTTGACCACCGAGCAAGAGATGACACAAAGCTTCAAAGTGGAACTGG
TGTTGCCAGCAAGAAACCTGGGTGGTCTAGGGGCTTCTCCACATCTCCCGAGTCATCGAAAGACTTAATCAGAGCTGGG
GACTGCCTGATCTGTTTGGTTTGGTTTGCTTTTTGTTTGTTTGTTTCATTTTGTTTTGTTTTGTTCCAAGACTTTCTTT
GTATAAACCCTGGCTGTCCTGGAACTAACTCTGCAGACCAGGCTGCCCTGGAACTCACAGAGATCGCCTGCCTCTGCCT
CCCAAGTGCTAGGATTAAAGGTGTGAGACACCACCATCCAGCTTTTATTTGTTGTTTTTGTTGTGTTGTTGTCGTCGTC
GTCGTTTATTTGTTTTTTAATGCAAGTCACCCCAGTAGATGGATTTCAGTTAAGACAGGAAATGTAAAAGACAAAAGAG
CTTTACACTGAAAAAGATGATGGTGGCTTGGACTGTGGAGGCGAAGGTGGAAGTAGAGGAGGTCGAGATAGCTTTTCCT
GGTGGACTTGTCCAGGCTTACCCACTGCACAGTATTCCCTGCAGTGAGATCCCTGGATTGTACGTGTATGGTTCTAAAG
AGACTCACATGCAGTGTAAGCCTTGTCTTATAACCCAGAACTGAATTCTCTTGAGCCAGACACAGGCCTGCAGATACAA
TCTCTGTCCCTGAGAAACATCCAGAGGCCAGGAGCTGCCCATCATCTGAAGCCCATCTTAGACCCACCCCTTCATGGTA
TCGCAACAATGGCAGCTGGGGTGGATGGAACACTTCCATGGCTCAAGCCCTGCCTTTTCTTTTTAGCTGGATGTCATTG
TCCTTTGAATAGGAGATAACAGCTTAAAATTTAATGTTAGCTTGTCAAAAGTTAAGAGACTTATAAAAGTCACTCAAAT
AGTAAGGGCTTTCCAACCTAGACTGTCACAGCTCAAAACCCATGTCGTTGCCAAAAGCCACATCAATGCCCTAGAAAAT
CCCACCAGAATTCTCAAATTTTTCATGTTTTTCTAATTTATAGAACAGAGACAGAATTGACAACATTCTTATTAGGGGA
CCTGAGATACATAAAACAGTAAATATTACTGTTTAGTTACCAAAATAAGATAATTGCTACCAAAAGAAAAAAAATCATG
GTTTCACTCATTTTCAGTTTGTCATCAGTGCCCCTTTATCACCAGCAGGGGCCGCTCAACTCCTTTTTAAAAATTTGTG
TCTGGTTCCTAAGCCGTCCTGTAGTCCTTCAGTGAACCAACCCAACGGAAGCTGCAAGTCCTGAGGTGCTGGCCCAGAA
GGGCAGCTGGGACTCAGATGATGGACACCCCCAACCTGCCTCGGCCTCTGAGCCAACCAGCTCTGGTTCCATGAGTGTG
CACTCTCAGAAGCATTCATTCAGATGAATGCTTCACCCTCTGATCTTGAGTCTTTCAATTGCTTGTTAAAAGGACCCAG
GTTTTCATCTTGCAATGGCTTCCAACTGTAGCTAGCTAATCCATGGCAGCCATCAGTCAGAGCGCTCCATGTTGACTGG
AGAGTGACTCAAATGTCACTACATTGGCAGGGTGGGCATTGAAGGTTCTATATCAGCAGACTTTAAAGCACAAGAAAAG
CAGGCAGGTCCCAGAGAAGCAGAGTGGAGAGCTAGGGCCAGCATCTTTGCTGTCACAGGGGGCTTGGGGGTCCTCTCAA
GATCTCCCAAGGTGGTAGCTGAATGAACTCAGGAAGACTTGCCTGATTGACCGTGGGGTGAGGGGCAGGTACAGAGATT
CTGTAAGCCCAGCAAGACAGGTGAGTGGACAGCTAAGAGCACTTGAGGCAAGGTAATCAATCATGTGACTCCGGGGCAC
GTCCTTACTGCAGTTTTTCCAGGCTCTTAGCCAATGACTATGTAACCTTCTGTAGCCCTGTCTCAGAGTAGACCCTTTG
ACCCAAATAAGGTAAGAAAAGCACGATGACAGCTGGGGACAGTGTTCAACTCCTCTGCCTATGACTGGTAGGTCAGTAG
TATAAAGACTGTCTGCCTATTTTTCTTTGGGTTGTTTTCTCAGTTGGCTAAACGTATGCAAAAACTATTTTTGTGACTC
AAATTTGGCCAAATCGTAGCAGTTTCCTTTGTTTTCAGCTAATGCTGTTCTGGGAGTTGATCGTCCATATGCAGAAAAA
TATGCACGCACGCACACTCATTAGCTGGATTTGCTGGGGGCGGGAGAAGGGGACAAAGGGGTGGAGAGCTCAAAAATAC
CAAATGACTGGAGAGGAAATGAGAGAGGGTGATGTAAAAGCCCATCCCTGTCTTCCCTGGTGACTCAGGCCCCCTTTAT
AGAGATGCCACCACCCGAGGGCAAAAAAGACTTTCCTCCACCCATACTCTGGTCTTGCCTTGGCTCTTCTGGCAGTTCT
GTCCCCAGCTTCCACTCCACCTGCCTTCCAAATGCTACTCATTAAGTGGCAGCCGTGCTGGCCCTGGAGGACTTTCTGC
CAAATCGCCAGTGAGCTTGCCCTTGACTGAGACCCCTCATGGTCTGATCCATCTTCTTTCAAGGCTCTGGTGTTCCCAG
CCTCTCTCCTCCCAGACTCTAGTCTCACGGTTGTGATGATTTTAGGAGATGTACCTACCCTCCATCCATCCCGCTCTCA
CCCGGGCTACCCCAGAGCTGGGAAGGTCAGGGACTTGTGCAAGTACAGGGCACCAAGGCACCTGAGCCTTGGGCTGGGT
TCAGGGCACTCATGGCAGAAGCAGATAGGGCTGGTCCCTACCCCCTGGGCTTTTCACTTTGTATCCTCAATCAATCAGC
CATCTCTCCGTGACCTGTTTTGTTTTGTTTTTTTCTGTCGCTTGCTTCTTTTATTTATTGTAATACTAGCATTTATCTT
CTTATCTAGAAGCATTCAACTTCTTCCCCTCTCTGCTTTCATTTTGCAAAGTCTCTGAAATAATACATTTTGTTGTTGT
TGTTGTTGTTGTTTTGTTTTGTTTTGTTTTTTCAAGACAGGGTTTCTCTGTGTAGCCCTGGCTGTCCTGGAACTCACTC
TGTAGACCAGGCTGGCCTCAAACTCAGAAATCCGCCTGCCTCTGCCTCCCGAGTGGAAACAATACATTTTATATCAAAT
TCACAGCTCATTTGTCTCCCTTAATAGTTGCTCATCTAACATTATATATGCATAAAATGTATGCACGGTAACTATATGC
ACAGACCTTGAAACCAAAGGACTCGAGTTCAAAACCCAAAGCCAAGCAACACTCCAGCTGCGCATCCCCTCACAGGCGA
TATTACCTTGCTAAGCCCAGGTCTCTGTGAAGCGAAGGTTGACAACACTACTATATCATACGAGTCTCAGGTTAAATGG
GGCAGTGCAGGTGATGGGTTTGGACAGATTGGGACCCATGGCAAATACCTGAAACCTGAGCTATTGCTTCTTATTAATA
TAATGCAGCAGGCACTTCAAAGGCATCCGATATTAAGCTCGTCCAGATTTTGGAGATGGCGAGGTTCAGCAGCAACGAC
TTAGAAGGCAGAAGACCTGGGTTTGAGCTCTCTCTTAAGGAAACTGTGCTGGGGGTTGAACCCAGGACCTTCCATTATG
CTCAGCAAGACTATATGGCTGAGCTCCAGCTCCAGCCTGAAAAGCCTCTGGTTTTAAGATCTAATTGACCCCTTGCTGA
CCTCGGGACCAGGAGGAGTCTGCCAACTTCTCTCAGCCTTCTGGAATCAAAGAGTAAAAATGCATTCAAAACTCCACTG
TCGCTTAAAGAATGATAAACATTTAAGTTAAGCATCCAGATTTTATTTCCTGCATAAATGTTCTGAAATTCATGTTTTA
ACAGCGCTGGATGTGTGTGCTAAGCTGATAGGTGCTTGTGCGGGCATAGCTGACTATGATTTGCATAAATATGTTTTCT
GCAGTCTGGAAGGAAAGACAACAAACCTACTGGCAAGGGTCACCTCCAGGGACAAGATTTGACAGGCTGAGGAAGGAAA
TTCCACAGCTATCCTTCAGAAAAAAGTCGTAGCTCTTTGGGTAATTTTTTTTCAGATAATCATATAAATGCTGCAAAAT
TTAGAAAAGTATTGTTTATTTTCATAAAAAACAAAACCAAAAACTCTTCTCACTGTTCTTGAAAGAAGTTCTGGGACAT
GGGTCAAAACTCTTATCTGTAGTCAGTAACTTCCGAGCAAGCGTCAGATCCACACAAAGCACTCTGGGAAATGCACGTA
TAGCTCTGAAGGTGGGGTTCGTGCCTGATAGTGCAGTGTGTGAGGCCACGTAAGTCACTCACATAGAAGAGCACAACGT
GGAATGTGTGCAGCACACAGCTAATAGAGCTCAGGAAATATCTGCTGGACCAATGGACTGCTGGGTGGATCTGTGTGTG
GCCGGCCACATCAAGTGCATGGATTCGTGGCTGGACCTGTGGATGGATAGGTAGGTGGACAGGTATGGACATGGAGACC
ATGCAAGAGGAGAGAAGGAAAGACAAAGGCCTCTGGCAGCTGAAGGCACTGGGGAACACTTGTGGGTGAGCCTTTGAGA
GGCCGACTGCGGACATTGCAGTTTGGAAGGGGTTGAGAAGGCGTCTTACAGTAATGGCACTCTGATTATTTCTCATTCA
GTCTCTTTCCCCTCCCTCTCTCCTCCTTCTCATTCAGGTGTCTTTAGAAGCATGGAGGACTTTTTTTTTTTTTAAGATT
TATTTATTTACGTATGTGAGCACACTGTTGCTGTCTTCAGACACACCAGAAGAGGGCATCAAATCCCATTACAGATGGT
TGTGAGCCGCTATGTGGGTGCTGGGACTTGAACCCTGGACCTCTGGAAGGGCAGACAATGCTCTTAACCACCGAGCTAG
CTCTCCAGACCCCAAACCCATCTTTTTAAGGCATTTGGATAACCAAAAAGTATGAAGTCATCAAAACTAATGACTGTTA
AGCTTTATCAACGGGGGAAAGGGCTTGAGAAAAATGAAAGAGGTGGTACAAGTTTTTAAAAAGATTCTCCAGGGGCTGG
TGAGATGGCTCAGCGGTTAAGAGCGCTGACTGCTCTTCCAAAGATCCTGAGTTCAAATCCCAGCAACCACATGGTGGCT
CACAACCATCCGTAACAAAATCTGATGCTCTCTTCTGGAGTGTCTGAAGACAGGTACAGTGTAGTTACATATAATAAAT
AAATAAATGTTTAAAAAAAAAAAAAAAAAAAAAAAAGATTCTCCAGGTTTAAACTATGGGAATTTGTGGGGGTGGGGGG
AGGACAGATTATTCAACAAATTATTCAAACTAAGCTAGCTAGGACAATTAGTTTTCCGTTGGTGGGGGTGGGGGAGGTG
TTCATCTAGATCCCTGGATCTTCCTCACATGACACTTGATCTTTCCCGGAAGACTGAGATGTAATTACACCTCACAATA
GAGTTAAAACAGATTTAAACATAAATTGTCTATAAAGGCATTTCGAGGGCTGGTGAGATGACTCAGAGGATATAGGTGC
TTGCCACTAAGGCTGATGATGTGAGTTCAATTCCCAGAACCCACTATGGAAGGGGAGAATTGACTCCTGAACGTTATCC
TTGGACCTTAACACTCAAGAGCGTGTGCACCTACGTATATCACATCATTGTCGTTGTCGTCGTCGTTGTCATCTTTAAA
GTAAAAGGATATATAAAATGTAGCTGTATCCTTTAAAAACTCTGAGGTAGGGGCTGGTGAGATGGCTCAACGGGTAAGA
GCACCCGACTGCTCTTCTGAAGGTCCTGAGTTCAAATCCCAGCAACCACATGGTGGCTCACAACCATCTATAAGGAGAT
CTGACTCCCTCTTCTAGAGTGTCTGAAGACAGCTACAGTGTACTTACATAGAATAAATAAATAAACATTTTTTAAAAAT
TTAAAAAGAAAAAAGAAAAAAAACCTCTGAGGTAGAGGTGCCTTCTTAAGCTACAAAAACTAAGAACTAGAAACTAGAA
AAGCAGATGAATATAAAATGCCTACATGACAAGGGATATAAAATTAGATGATGGCTCCATATATAATAGCTGAGGATTT
TCTCCAAGTAACACATAAAAACCTCAGCAAATCAAGTAGAAAAACAGGACCCCGGTAGAAAAGCAGGCAAGGGGTAGAA
TTGAGCAATTCACTGGGAAAAAAAAAAAAGGATACAAAATACAAAAGTATGATTATGTCACGAATTGCAGACTGGCTCA
GGCCATGCTCGGCATCCTGCAGCTTCCCTTCCTACACGCTGGACGCTGGAATGTGGACAGCAAAAGTTTCCAAGTCCAG
TCTCCTTAGTGCTGAGCTTTTGTGTATGGCTTGATACATTCTGATGTGTCCATCAAAAGCCACCAGCTCCACTGTCAAA
CAAGGTCTGTGTGTGTGTGTGTGTGTGTGTGTGTGTGTGTGTGTGTGTGTGTGTGTTACCTTGCATCTATATGCGGACT
CAAGATCCATGGGTATCCCAGACAGGAAGTAGGCATCCATTGTATAGCCCAGAGAGCATGGTCCTAGAGACCAGATATG
CCTGACTGAGGCTTCCTGGCTTCCAGACGGAGAGGTCTGTGATTAGGTCCTAGTTTCTCAAGTCCCCAGGTTACGGTTC
ATGGCAGAAGGAGAAGCCACGTCTGTGGGTCAGTCAGTAGCTGAGATTCCAACATGGAATCTCAACCTGGAACCTTCCA
TTCCACCTGATTTGACATCATTTTAATAGATTGATTTTCAATAATAAATCTCTTTCCCTGTGTTCTACCTGAAGTGGCT
TCTGTCATCTGAAGCTGAGCTAAGACGCATTATGGATAATTTAAAAAAAATACAGATTATCAACAATGCTTCATCAGAT
GTCAACCCTTTAAAAGATTAGCAGAGAGCCCCACACAGGCTGTCATTCCTGGAAGCCATGTCTTGAAGCAGCCATTTTC
TAGTAGTTTGGTATCGTGCACCTAGACATAGCAAGTTCATCTCATCAAGGATTGTACCTAAAGGATGTTGTCAAAGAGA
AACAGGACACATGTCCTTCAGGAAAGACTTGCCAGGGGGTCTCAGTTGCATCACTGTGAGGAATGGAGAAATGATCATA
AAGGAAAAGCTGAAGTCTCCATCCAGAGGGGATTGCCTAAAGAATTATTGTATAACTATTCCTGGGAATTCTTTGCAGG
GGTCGAGGAACAAGTCAGAGCTACCTACATGAGCAAGGGAACGTCATTACATTACATTACAAGTAAGAAGTAATTTGGT
TATCAGCGCGCAGAGCGCGACCTCGTTCATGACTGAAAACATGAATTTTTGTCTGAACACGGGTGTGTTTGCCTAAACG
TGGAGAAAGGTGAGAGAGGACACAAGGGATGATGATGGTGGCAGAAGAGCCCACGCGTGGCTTTCGCTTATCCTCTCTC
TAGAGTCTAAGCTCTCCTGTCTGACTGTTCTATGAGATTATGCTCATATGACACATAAGTATACAGTTCAACTCTCTAA
GGAGCAAGATTCAAATTTTCCAGTGTCTAAGAAAAAGATATGTGCGGATGTTTTCTAGAGGAAGCCGGGAGAAAGGGGA
AGCAGGATAGAATGTAGCGTTTCCTGAATCTAGGCCGAAGGACATGAATGCTCAGTGAGCTAGTCTTAGGACCTGGATT
CTTTTCATAACGTAGGAGAAGCTCACATTAAGTAAAAATCCTTGGACACACGCGTTTTTCCTGCATAAGCAGATTTTTC
CAGATGTGGAAGCAAAGCTTGTGGCTTGCTCAGCTGTAATTATGGCCAAGAGTTATTGGTATTCCTTCTTTCCCTCCCC
CACCCCAGCTGCTTAGGGTAAGCGTGTCCTCACCCCCGTGGCCGGTGTCTTGCAGAGGCCACAGTGTACAGTGACTGGG
TGGACAGACAGTGCCTCTGCAGCTAGGCCCCGACGGGAGCAGAGACCAAGCTGACCTTCTGGTCCCTCTTTTTTGACCC
TTTTAATTGTACACACATCTCCCAGGTGTATCTGGACTCTAGCTGGTGACAGTACCCAGAATGCTTGTCAAAAGAAAAC
ATGACTGTTCCTAGCAGCCCCTGAAGAGCCAGGTGGCACTGTCAGGATGACTTGGAGACCCCTTCCTGACCATCCCGAG
GGCTCTGAAAGGACTCTATTCAGTGGGCTTTGCTCCTTAACCTGGTGTTTATTTTTGAGATAGCAGTTTGAGAAGAATG
GGGATGGGGGGGGCGGAGAATTGCAAAAGGGGGGCAATGAGGGGCCTGGAATTTTAATGAGAACCATCTGGAGTGGTTT
CCAAGAGCGTAATGTTTGGTACAAGAAAGTAATTCCTCGGCTCAAATGGAGAGAGGAGAAATGGAAGAGATCGTTATAT
TGTGTTTACCCTCCAAATGGTGCCAAACAGCGCTGCTGAGACCGGGCAAGTCACCCTTGAGTAGGGCTGGGGTGGGGGC
TCTGGGAGGGGACCAGCAGCGTCTCTTGGCTCTGATGTGGCTGCAGGGAGAAGGAATGCTTTATTTCTGCCAGGGAGAC
TTTGAGGGATGGGTTGTGGTGGGCCCAGAGCCAGTCCCTGGAATCTGGGTAACTATGGAAACGGGCTGAGGACTACAAC
AAACACGGATGGAATTTCAGGCTTTACAGCCAGGGCAGGGAGGAACGGGAAAGTTCGGGAAAGTTTCGCTCCCTTCTTT
TTCTTCTCTCTTTTCCTTTCCATTCCTTTCTGCTTTCCTTTCCTTTCCTTTTTTTTTTTTTAAACGGAGGAGGAAGGGA
GCAGCCCCCCGCCTTGCCCCTGCACCCTAACGGAGGGACCATAAGCAAAAGTCTCTACAAGTATCAACTTTTCACTTTA
TTCTTTCATTTACTCTTAACCTGTTGCATCTGATCGGGTATTCTGTCGCCACTAAAGCCACTCCTAGAGGGAGTCTCTG
TCCACTGTCTAACAGCATCATAAGGACTGACATCCCCCTTGGGGCCTCCAGTGTGACCCAGGAGGCCCCAAGGAGGCTC
GGTTCATCCTCACCATAGCCCTGTAGACCAGATGGTAGGGTCTCTGCCCTACAGATGGGGTAAAGAAAACTCAGAGCCA
AGATCACCCAGAAAGCAGAGACCAGCTGCCTAGATCCAAATCTGGCATGCCGTCATTCTGACAAACTTTATTCTGAGAA
AAAAAAATTAATAATATGGCAGCCAAAGAGAATAAAGCAGCTTCCAGTGTCTAAATGGCTGCAGAAGTGGCTCGTGCTC
TCACCTCAAAGATATGGCCTCAAGATGGCTGAGGGAAAATATCTGTGGCGGAAATAGGGGACGTTTAGAGTTACTGAAC
AGTGAAGTTAAAGAAAAGACCATAAATCCTGGAATCATCTTCTTGGTCTTAACCTAGGAGCAGAATAGCCAAGGTCTAT
TTAGATGTGATAATAAATGGGCCTTCATCCTCCAGATGAAGAAAACAAGACTCAACCAGAAGTTCCAGCCCGGACTCCT
GGCTTCCAATACAAAGTTGTTTTCTAGTGTGAGCCATGAGTCTAGAAAGAGTGGAGACCTTGTGACTGGGCTGGGTGAA
GTCAGATGTGTCTCTGGGTCAAAATGGAGAGTGGTAACCACTCTGAAGAGCAGAGGTCTGAGTCTGGGGTCAAAGGAGG
AAACAGGACAAAGTGGACAGGCATTCTTCAAGACTCATCAGTGTGGTTTCAAGTCCATTCAAATTCCCCATCAGGAGCT
AGGAGGGAGCCTCTGTTCAGCTAAGGATTTAACAAGAAGATCAGCCTAAGTCAGCAAAGTCACAAAACCTTTCAAGAAA
CTTGCAGCTTCACTGGGGGAAGGGGCTCCTGAATAAACCAGCAGGAAGTCAGAGTGCAGAAATGTCTGGAAATGAGAGA
TGGTTTTTGGCTGGTTGGTTGATTGGTTGGTTGGTTGGTTTTTTAAATAGAAAAAAAAAAGTTTTGAAGGAAAAAGGAA
GCCTTGACCCACATAAAGATAGAGTTTCAGTAAAACTAACTTGAGAGGAAGAGGGCCAGTGCGAGAGGCTCAGCAAGGC
AGCGCTGGCCAAGCCCTTGGGGCTCTCCACACATTAAAGGCAAACCTTTACCCAAATGCATTCAGTCTGCAGGAGATTG
TCCATGGCCCTGTTGAAGTTTGTGAAAAAATTTAAAGATTGCTTTAGTGTTTGGCAATTCTGGAATGCTTGGGATTTAT
CCTGAAATAATTGATTTTCTCTAAAGCTCTGCTTCGTAAAATTGTATCCTGAAAACCCCCTGGAGGGTGTGTCACTACA
GTCGAAACTATTTATGTGTTTGCATATATGGGGGAGGGTCTGCTTGCATGTGTGTGTGTGTGTGTGTGTGTGTGTGTGT
GTGTGTGTGTGTGTGAGTCTGTATGTGTGTGTGTCTGAGTATGTGTGGTGTGTGTGTGTATGTGAGTCTGAGTGTGTGT
GTGAGACTGGTGTGTGTGTGTGTGTGTGTGTGTGTGTGTGTGTGTGTGTGTGTGTGTGAGTCTGTGTGTGTGTATCTGA
GTGTGTGAGTCTGTGTGTGTGTATCTGAGTGTGTGAGTCTGTGTGTGTGTATCTGAGTGTGTGAGTCTGTGTGTGTGTC
TGAGTGTGAGTCTATGTGTGTGTGTCTGAGTGTGAGTCTGTGCGTGTGTGTCTGAGTGTGAGTCTATGTGTGTGTGTGA
GTGTGTGAATCTGTGTGTGTGTGTGTGTGTGTGTACGTGCCTATGCCACATATCCCACATATGAACTCCAGGCGTTTCT
CTCAGAATCTGTCCACCTTGTTTTTTGAGACAGGGTGTCTCTTACTGGGACTCTGGCCAGCCTAGAGTGGTTGACAGAC
ATGCTTCAGGGATCCTCCTTTCTCCACCTTCCCAGTTCTGGGATTAAAGGTCCCAGTCCTCCCCAGTTCTGGGATTAAA
TGTCCCAGTCCTCCCCAGTTCTGGGATTAAAGGTCCCAGTTTATGTGTGCACAGGGGTAGGGGCTGCTTATGTGTGGAG
TATATATGTGCATATGTACCAGTGCATGTGGCCACGTTTGTACATGGAGCCCCCGGGAATATATCCTGTGTCCTCCACC
TTTTTTTCCTTGAGACAGGGTCTCCCACTGAGCCTGGAGTTAGGCTGCTAGCCAGAGACAGGAAGCTCCCCCAATCTTC
CTCCTCTCTCCACCCCTAACAGTGCCAGATTTGCAAGCACGTGTGTTGCCATGCCTGGGTTTTTATGTGAACGCCAGAG
ATCAAATCCAGGTCTTCACACTACCAACTGGGCTCTCTCCCCATCTTCTCGGCAAACATATTTTTTAAACTTATATTGT
ATATTTATTGCTGTGTATATGTGTGGGCATTCCATCAGAGGGCAACTTGAGGAGAGTCGGTTTTCTTCTTCTACCTGAT
GGGTACCAGGATTGAGCTCAAGTTGTCAAGCTTAGACGCAGTGTCTTTACCTGCCAAGCCATCTCGCCAGCTCAACAGA
TATTTTTAACAAGATGCCAACCCCTAAACAAGGTGCTATTAAGTTATCAGATTATTGCCTCTTGGGCTGAAGGCTCTAT
CTGAGTTTCTAATATTAAAATACAGAATGTGCTGTGAACCCATGCTGAGTTTTCCACCTTCAGATGTCACAAGTTCCTC
ACAAGCTGCAGAAGGCCACTGTTCTTTCCTAATGGGACTCTTCTGTCTATGATTCACGGATTAAACTCCTATAAAGGGT
TTTATGTACATGCTCAAAATATGTGGATCTGCAAGAAGGCTAGGAGTGCTCTGGTAAGTGTGTATCTGCTCGTGAGAAA
GTGTGCGTGGGAGGAAAGGCTGGAGTAGAAACTGCTTTCATTTTAAAAAGCCGGGAGGGTAAGCAAAATCTACTGATAG
ACATCATCAAAGAAGCAAGAACACAGGGCACAAAGTGGGAAGAAAGCCTGTGTTCTCAATGGCCATTCCTTTCATCCTT
TAAGGCGGTGGCTCTCAGCCTTCCTAACGCTGCGACCCTTAAATACAGGTCCTCGTGTTGTGGTGACCCCCCAACCATA
CAATTATTCTCATTGTTACTTTTATAATTTTGTTACAGTTGGGAATCTTAATGTAAATATCTATGTTTCCCAATGGTCT
TAGAGGACCGCTGTGAAAAGGTCATTTGACCCCTTAAAGGTGTCATGACCCATCGATTGAGAACCACTGCTTCAAGGCC
ATTTTTCCTGATGAGAAACTGGGCTGATGGTAGCCTGTACTGAGAGAAGGGTTCCGACTGCCGGTGGGCTCCACACCTG
TACACCACACTCATGGGGTGCCAGTCTGACTGAGAGGGAACTGTAGTACCCAGTGTGCCCACGTGATTGTCATGAGGAA
CAAACTTGTTCTCCAAGCCAAAGTGCCCTTTCTGGTCAATTCCAGACTGTTTCATAGGATGAGCCAAAGCTTGAGTACA
GACATGCTGTCTGGAAGTCACGATCACACTCCAGACTTCTTCATCTCCCCATCACTGCTGGGTCCCTATGGCTCTCCTC
CAGCTCTCCTGAGTGAGGCTATTAAGCATCACTTCTGTCCCCACCCAGATGCCTACACCAGGCCAGAGTTTCTATCGTC
TCTGATGAAAGTTCCATAGCTGAGCTCCACTTGGCACTGGGTCATCCATGACAGATGATCAACAAAGAAGCCAGTGTCC
AGGAAGTGGGGAGGTGGAATGCCATTGTACCTTTTGCTTTGGTTAGTGACTTAAACACTACAGGGAACTGTTATTTCGA
ATGATCTACCCAGCTCCAGGGTTGGTTAATCCAGCATCTAAATGATATCTCAGAGGTCCTTGTCTGTCTTTAAGGGTTT
TTTGGTTTTTGGTTTTTGGTTTTTGGTTTGGTTTGGTTTGGTTTGGTTTGGTTTGGTTTGGTTTGGTTTTGGTTTTGGT
TTGGTTTGGTGGTTGTGTTTTTGTTTTGTTTTGGGGGATTGTTTTGGTTGGTTTGGTTTGGTTTGGTTTGGTTTAGTTT
GGTTTAGTTTGGTTTGGTTTTTTTGCGACAAGGTTTCTCTGTGTAGCCCTGGCTGTACGGAGCTATCTCTTTAGATCAG
GCTGGTCTCAAACTTAGAGATCCATCTGCCTCTGCCTCCCAAGAGCTGGGATTTAAGGGTGTGCACTACCACTGCCCAT
AGGTTTTTGTGTCCTGTATTCATCACTTTGTACAGTCATGGTTATAGGATGCTGTCCACAGCTCTGGGCAGCCCCTGCT
CACCTCGGAACAGAAAGGGGCCTTTCTTTTCCCTGTGGTTCACCTATAAGCAGATGGCCAAATCTGGGCCATTGAGAAC
CTAGCTTTGTGGACATCAGGCAGTCATCTTGGGGCTGTGTTGTGGTGACATATCTAAGAAGGGCTGAAGTGTTTGAGAG
GAAGTTAAGTCAAGAAACAGGACATACCTGTGTGCAAATGACAACCTGAGCCCATAAAAGCTTCTGGACTTCTTCAGTG
GTCCAAAAGCCTCTTCCTCAACTTGGTTGCCTGTCACTTGTGTCCCTGTCACTTGTGTCCCTGAGGACCTACTCCTGGG
CCTCACTTGTCAAAATAAAAATCCACTGACCCTAAGCTCCAATGCCCATGTAGAGATTGGATGAGAACCTTTTGGAGTT
GCTTCTTTCCTTCCTCCATGTGGACTCCAGAGACCTAACTCAGCTCAGCAGGCTTGGCAGCAAGCCTCTTTACCTGCTG
AGCCATCTCCCCGGCCCCCAGCGGCTCCTCCTCTTTAGAACTATTGAGGCTGTGATTTAATAAGAAAGGCGAGACCTGG
TCACAAGATAAGCAGACCGGACCACTCGCTATATGTGAACCGACATCCTGGGCCATTCCTCAGGTAGGAAATGAATCCT
GGGGAAGGGAGTAGTGAAATTCAAATCAACAGTCCAGTTTACAGGACTGCATCTGCGTTCATCTCTGTCTTTTGGCAAA
CGCACTGAGCTTTCTATGAGATGTAGAAGTCGGAGCGGCTCAGTTGGGAAAGTGCTTGCTGTGAAATCGTGTGGGCCCG
AGTTTGATCCCTTGAACCCACAAAAGTTGGATGTGGTCACACATGCCTGGAATCTCAGCTCTGGAATGTTGATCTTGGA
ATTGCAGAGAAATGGAAGAGCCTTGGTGATCCCTGGCTGGCCATTCTAGCCAGATCAGAGCACAAGGCCAGTGAGAGAC
CCTGTCTCCAAAAGAGCAAAGCCAGAACCAACCAACCAGACCAAGATGGATAGATAGTTCCTGAGACATGATACCCAAA
GCTGAACTCTGGCCTCCACACGTGCAAACACACATACATGAGTACACACACATGCACACAGACACACAATGATATTAAA
GACATAAAGGAAGCTGAATAAAAGCTAACATTAGGGAAAGATGCCTGGTTTTGTAACTTTCCTGTGAATCTCAAATTGT
TCCAATTTTTTAAGAACTTTTAATGCTTTATTAGTCATTTATCTCTAGTAAGTGCTAGATCAATCAGACTTCTTCAGCT
CGAAGTTAACACTAAGTCCCAAGGAGGCTAAGGACCTGTTCATCGCACCTGGCAATCCGTGTCAGAGCTGGACCTAGGA
CCAAGTCTTGTCACCTGAGTGGATGATTGATTCATGTAAGCCCAGGGATGTAAGCCAGGCCTTTCTCACAGGACACTGT
TCCCATGGAAACAGAGCTTTTCTAAACATACTCAACTCCCTTGTGGCTCTCACCACATTAGCTCTGGATTACTGCATTT
CAAAGTCCATTTTTCACATTGATAAATTGCACAATTATAGGCGTCCAAGAATGAGCCGTAGCCTCATGCAGATGGAATT
TGTACTAGAAATTTACACTGCCATACGGCCAGCGGATTAACATTAATCGTAAATGGAAGGCAACACGCTCTCTGGTGCT
TGCCAGTCACATCCCTCTGAATTAAGTGATTTCTGAAAGGCGATAGTCGAACATTGTTGATGCCGAATGAACCAGAGCC
ACGGTATTTCCTAAGGACATTTTAAAGGGGTTATGCTCCGAGGCACGTAGGTGGGGGTGACATACTCAGGGAAAAACGC
TATATTAGATGATTCAAGAAGTGGCTTTAGCGATGACAGACGCTGATGTCAAAGACTCATTTGTAAAGTGTGGGTAACA
TGTGTCCCATAAAATGTGAAGGAAAGAAATGAGATCCACGCCATCAGGGGCCATCGCCTGTTTTCGAGAATTCTGCTGT
TTAGTGTTTCATAACCCAGTGTCAGGATGAGACGATGTTCCCAAATGAATGAAAGAGAGAGTCCCTGATCTGCTTCCTG
TCCCTAAATCCATCCACTCCTATTCTTCAGTGTCCCCTCCTGCCAAGTTCTGTCATCCGTGCCTCCCGACCATGTGCCA
GGAGCAGTGTCATCCACAAGTGTGAGATTCCCTCCTTTCCTTTCCCACCCAATAATTACCCCTCTCCCATCCTGTGAGT
GCCAGGCCCTTGGGCAGAGTCCAGGCATCCTGGAATGCTGACCGCAGATGTCAGAACCCAGGGGATCCCTGAGAAACGC
AGACAGGGATGGCTAATGAGCACTCTCCCCTGTAATTGCCCTGACGATTGGAATTATCTGATCAGAGCTACGCCACTTC
TCTTCCTCAGCACAGAGATGACTTGGGACGCACCTCTCAATGGAGGGGAAAGCCACAAGTTCTGATGTTTGCCACACCA
TCCTTCATTAGTTAATGGTTATGTGACAGATGACTTTAAAAAAAAAGAAATGATCTTGATAGACAAGAGTGATGTGCCC
CAAACTGTAAAAGGTAATTTACAAAGAATGTAGGCATTCCCTACAGTTAAAGTTACAGAAGTGCCCTGAAGGGGCTCAG
AGGACAGCATGTGACAGCAGCTAGAGCTGAGATCCAAGCCTTGGGAGCCACAGGTTCTGTCCCAGCCAGCTCTCCAACC
AGCTGGCCATACTCCACTTAATGATTGGCTTCCCTGGACCTTAGCTTCTTCATCTGAAAATGATCATCCATGTACTCTC
CAAATGCAGAACAGTCTCAGCCAAAAACAGGAGAGCCGGATAAATTGTGGAGCATCCTCATGACAGAACAGCACGCAGC
TGCTATGAGCCCCATTTCCCAAGAACACTTGATAATGTGAGAAAGTGCTTGCTACACGGTGGGAATTTAAATGAAAAAT
AGGAGGGCGCAAACTAAGTGTGGCCAACAGGCTCTAATGATACAAAAGGAAATGCAGAAGAGGGAAATTGCAGGGGAGG
GGGTGAGGCGAGGGGTGGGGGGCAGGCCAAACGTGTTAGTACAGAAAGTACTAACAGTGTTTACTATGGGAAGTTGGCA
TTAGTTTTTATGACTCAAAGAACTTAAAATTGTGCTTATTATTTCAAAAGAAGCTAGTGCTTTGTTAAGATTCCAACAA
TACACAAGCATAAAGAGGAGAAATTATAGACCCTGGGTTTCATCCTCCAGCATGGAGGAGGTGGAAAAAGAGGAGGAAA
GGATAAATGGAGGTGAGAGAAGAAAGAAGCAGGGAGAGGAGGGGAAAGGGAGGAGAGAGAAGAGGAAGAGGAGGGAGAG
GAGGAGGAGGAGGGGGGAGAGAAGGAGAAGGAAAAGAATACAGTGGGCCCTACTTATGATTTGAGAGGTATGGAAGAGA
GGAGGGAAGAGAGAGGAGGGAAGAGGAGAATCCATACATAGACATTCCTACTCACTTGAAGTTACTGCATTTCTTTATT
AAATATATGCCCATAAAGTAAAGAATATGAACTTCAAGTATAAATTTTAAAAACATACAAACTGAAACTTAAGTCAAGT
AAGCTTGGGGCAGGGGAAGGAGAAGCTGCTGTGTCAAATGCAGTTCTTGAGGCCCATAGAGCGAGCCGGGGGGCGGGGG
GGAGAGGAGCCGAAGATCTGCCAGGCATGGCCGCTCAGTGCTCACTTACAAAGAAGCCAGTTGTGGACTCCAGCATTAC
AGAGGAATGAAATGTCCCAAAGACGATACTCGGTACCCAGGCTCAACCTCCTGCTCCTTGGCAGAAGAAACCTCAGTGC
CCATCCTGCTACACTACCCACAGAGATGTACCTACGAGGACCCTAACTAACCAGGTCAGGCAACTACCATGGCAAGCCA
GAGGGTTGAGCGAGCTAGAGTGCAAAGATTCACCATTTCTGGAAATAGACTCCCTAGCTTCAGAAGTTGAAAGCGGACA
CCAGGGCCAATGAGCCCCAGCAGTAAATGTTCCTTCCACACAAACCTACCTGACCAGCTGAGTTTAACCCCAGGAACAC
ACAGAAGTTCAGATACAGGTTCTCACATCTGTGATCCCGGCACTTCTATGGGAGATGGGTGTCAGAGATGGGGGAATCT
CCAGGAAGCTAGTGGGCTAGGTAACCTGGAGCTCAAAGAGTGGCAGAAACAGTAAGAGAGACCCTGCCTTGACCAGGTA
AAAGGACAAAATCAACTCCTGAGAGCTCCACGTGTATACCCAGATGCACTCACATAACCGTAACTAGCTTCATGTTTTA
AATCTACACCAATTCTAATAAAGTAAAAGGGAAACTAAATTGTCAAAAAGATTCAAATCAACTCTGTGGGTGGGTGTGG
AGGTTAAATTCCTGAGCTTGTTGAAGAACTCACAGCTCACATCTGTAACCTTGGTCATTTTGTCTTTCTGTTTGTCTGG
TTGCCTGTCTGTCTATCTGTCTTTTTAAGACAGGGGCTCTATATATAGCCCTGAATGTCCTGGAACTCACTTGGTAGAC
CAGGCTAGCATCCTGGAACTCACTTGATAGACCAGGCTAGCATCCTGGAACTCACTCTGTAAACCAGGCTGGTCTTGAA
ATCATAGGGTTCCTTCTGCCTATGCCTCCTGAGTGCCAAGATTAAAAGCCTGTTCCCAGCTCAATCTTGGCCATTCTAA
GTAATTGAAATTCTAGGAAGATTACTTAAATCATACAGTAAAATGAACGTAAGAGTGAGAACAGATGACTTCAAATACT
GATTCTTTCTCAAATCTTTAGATTTTCTTTTCTATGTATAGTGTTTGCCTGCCTGTGTGTCTGTGTACCATGTGCATGC
AGTACCTGTGGAGACCAGAGGAGGGTGTCATATCCCCGGTAACTGGAGTCACAGAGAGCTGTGTGGGTGCTGGGAATCA
AATCCACGTCCTCTGGAAAAGCTGCCAGTGTTCTTAACCACGGAGCCATCATTCCATCTCCAACCAGCACTTTCTTGAA
TGGCTTTAATATGAGTTTAATAATCAGCCTTACAGATACTGGGCTATCTGGGGTGGTTTTGTTTTGTGATGTGCTTAAG
GAATGTAAGACAATTTGATAGGATAGATTGTAATTATAGGATTACAATGTCATAAATGGCAGGAAACTAAAGAGTTTTC
AACCAAAGGGAAAGTAATTGAAGATTTAGGTGAGTTCCAGTTATCAATGGTATAACTGTTACATGAATGTGCAGAGAAA
TAAGCTGCACTTTCAGTTAGCAGAACACAAATCTTTGATAAGTTCAGAATATTGGTGAGTAGATCATTAAACAGAGGAC
AAGAATAGTAATTATCCTCCATCCTCAAAAGCCCTCGGACATAAAAGGCTTAAACACAGCTTGAAACAGCAGCTGTACA
TGCTGAGGACTCCAGTGACCAAGGTCTCTGGGCTCCATAGCACCAAGGTATAGGAAGGAAAAAGACAGAGAACTAACCT
CTATCTTAACCAGAGAAACCCCTGCCTCTGTCAGAGGCAGCACCCTCTACAAGAGGCCAGGCCCAAATGGGGTGACACC
CCTCTGCTGTGAGGACAATGGGGTGACACCCCTCTGCTGTGAGGACAATGGGGTGACACCCCTCTGCTGTGAGGACAAT
GGGGTGACACCCCTCTGCTGTGAGGACAAAGGTGGCACCTGCTCTGGATAGGTTCCAGAATAGGTCCGAGATCTATCTC
CGCCCCAGCACCGTGCAGTGTTTCCACCAAAGATGCCACATTCGTTAACATTTGAACCCTTCTTGGTTCATCCAGGCGA
GGTGGAACCAGGAAGAAGGAAAAGATGAGAAGCTTGGGGTCGTTAGTTAGGACCCCACATGGAGAGATTTGACCTCACT
CGTATCTCCCATACTGTTGAAGAATGGCAAATTGGTTGCTTTCAAATCTGTGGTTGAACTTGCATGCTATTGTTTACCC
ACAAAGCTTTTATTCTTTTTTTTTTTCCTTTCCAGGTGTCTGGTACATTTCCATACAGGACAACCTGAGCTCTGTGCTC
AGAACACCTGTTCCTGTCCCAGCGGATGTCCCCCCACCCCAACCTCAACCCACCCCACCCCACCCCCATTCCACCAGCA
GGAATATGAGCAGATCAGCCACAGTCAAGAGTGTCACAATGAAATTCTGATGAAGTGTGGAGCTTTGGGGCTAGGTTAA
ACTCTACCCAGAGGGAGACTGGGAGAAGCCAGCCTAGGACCAGGCCTGCAGGATTCCCCATCTTTTCAATTGCACAAGC
CAAGAATTCCCCTTGGTGGCTCAAACCAATTTGATTGGACCTTAGCAGGAGTTTCTGGGAGGCTTTTGAGAATCTGGAA
ATATCTCTGGTGGTGCTGTCCTAAGGAAGAGAGAGTTGTAAGACTCTCTTCAGACATAACTTTGATGAAGAGAAAGGGG
CAAGTGAGCTTTCTAAAAACTAAAGCTGTCTTGCAGAAGAACAACCTGCCTCAGGAGAGGAAGTTATCTCCTTAGTGAG
ATAATGCAGGCAAGGTTGGATGAGGTCCTGACAAGGATGTTGGCACAGAGGTTCCTTATTTGAGGGCGTGTGGATGGCA
AACGGCGCCTTTTACTGTAACCCGCAACAGAAAGTAGAGGAGACAATCCCAAGGGGAAATGGGTGGGGCAAACGGACTT
GGGAAGCTCTAGAGAGCCTCAGTTTGGCCAATGAGAAGAGGTGATATTAGCTCTTAGAATCTGACATGGTCCTTACTGT
CACTGCTCAGTGACATGGTTCAAGTCAAAGGCATTTCAGGGAAGTATGACTTCCACATAACATGAGCTGTTTGGTGTGC
ATTCTCTAGTTTTAAAGCCGCTTCTCTCCCATGCACTTGGCTAATATGAGGGTGAACTGTGGAGGAAGCCACCCACATG
CTTGAAGAGGGTGAGGGATTCTAGCTATACCTGCACGCTTATGAGACCAGGGCTAAAGTCCTTTCAGGTCTCTCAGCCC
ATCAGCACTGGGTGTTCCTCACAGATAAGCAACTAGATTAGGCATTGCAAGGCTGAGATGCCACAGAACCAGTCATGTT
GGCATTAGCAGGTTGGTGAGTCTCCCTATCATCTGCCTTTCTTCTGTGACTTCATCATCCATCCATTCCCCACCCCACC
CCCACCCCGAATTCATCCGTACTTTGGTCACTCGGTGTCCTTTTCCTCAAAGGCGTCTTTTCTTCAAAGACACCTTGCT
CATTCCTGGTAACTCCTGTGCTCCTACCTACCAGGAAAACACACCACACACACACACACACACACCACCACCACCACCA
CCACCACCACCACCACCACCACCACCACTACCACCACCACCTACAACCAGGTTCTTGAAGTTGTTCAGCTATGAGCAGG
GATTATATCTCCTTAATGAGTTCTCTCCTGACAGCCAAGCCACATTAGCCAGCTGTGTTTCAACCCAATCCATCAGAGT
ATCCTGATCCATTCACTTCCTAAAACTCTTCACTACGAATACTGTTGTTTCTATTTAACGCTCCCCCCACTCCATTAAA
ACACTCAATAAAAACAGAGGCCATATCTGTTCGGCTCTGCCACCTTCTCAGCACTTAGAAGAATTCCTGGCACGTGGTT
TTCATAATCATGAGTGATTGTTAAATCAATGAATGGCTAAATAGATATATTCATTCTTGCCTTGACCTTAGAGACATTC
CCGCACTCTATTTTCTGTGAGTGTATAAATAAATGAAATTCAGAGGTAGGTGGGTACTCTTGTTTAAAATACTCATCCC
TCATATAGAACTTGCCTTTTAATTGACTCACGGTCCTGGAATAATCAACAGAAAGGGAACACAGAACCTTCATTCATGG
CCCAGCCCCTTTTGTCAGTATCCCTAGCACACTTTGCAAGTGCCAGCCAGCCTTATTTTTATTGGGCAATAACCTAGCA
TGGGTAGCCTAGTGCCTCGCCAGTTCAGTCTGCCTGGTGAAGACAGCAAACCCAACACAGCCTCCCTCTTCCACCTCCG
GGAAAAGCTAGCAAGTCTTGAGTGGAACTTGACTCTCCGGTATACCCATTGTTTCTCTTCCCACTATTGACAAATAGTG
ACTTGCCAGCCTGCCCAGTGCCTGAGATAATTGCCAGAAGATGGTTTTTAAGTTATGAGTGCATGTCATTAAAGCTGGG
TTTGAACCACCCTCCAGAGGACTTTGCCCCCTGGGGGGATTTCTTTCTGAACCTAGGAGGAAGAGAGACCTGAAATTAT
TTCAATGAAAGAAGGCAGGGAGGAACGCTCTGGAATGTCACCTCACAGGCTTTCCTGGAGTTAGGTGTAATCTCAAGTA
CCCTAAATATGTTGCTTAAGTGGCTACAGCTGGCTTCAGCCTCTTGGGGAAGTCTGTATGCAGGGGATGTGGCAACCAG
CTTCCCACGCGTGGGGAGAAAGGCAAAGCACCTAGTTTACAGGAACAAGATCTCAAGGTCGAGGCCACTTGGACCCTGC
CCTTTAAAAAAAAGATCAAGGGTCATTTGCTTCGGGCTAAAATACTGAATCTGGTGGGATGGCATTTATTTCTTGGTTT
GTTTTTATAGTTGCTGGAAAAAAAAAAAAAACAGACTGTGGTCCACCCCCTCTCACTTCTCGCTTACTCACCGACCAGT
AGGTTTGATTACTTGTGTTTTAGATACCATCTCCGGCACCAGGCAATGGACTCTGTTTTGGTTTTTGCTTTTGAGACAG
AGCCTCACTGTATAGCAATGGCTGGCCTGGAACTCACTCTATAGACCGGGCTGCCTGCCTCTGCCTCCTGAGTGCTGGG
ATTCAAGGCCACCACCCTGGATTAAAGAGCAAAATTGGTCCACGCAGGGATGGACCCACAGCGTTGCTGTCATTGTTAC
TATTAGCCATTCTAACCTACTGATATAGCCACACTGCCAGGCTCCCCTTGAAGGGGAACAGTGACCACTTCCCCAGCCA
ACCGACCTCCTGCTACTCCTGTGGAAACTCTTCCTCACATCCTCCTCTGTTGCCATGGTTCCGAGCCAAGGTGAGCATC
TCCACCAGAACAGCCTGGGTCCCCCAGGAGAGACAGTAAGGGAGATAAAGGTAAGACAGACACAACAGGACTTGGTCAG
GCTCCTTGAGTTGGGGAGATCTCAGGAACATTGTTCAATGGTCCCAGTTACCGTGGCTCAATGGACCAGTAAGGGCAGG
AAAACAGAGACTAGAAAGACAGCAAGCCCTACCCAGGTGGTGCTGCCCAAAGAGAAGGAAAGGGGATCGGTGTCACAGA
CCACCTCAGTGAATGAACCAGCGTGGGAGATTCAAGTAAGCCAAAGCCGCCTGATTCTCGTACCACAGGCAGCTCTGCA
CATAGTCTAGCATCTTGTCTTAGCCATAGAACAGAACACTGGAAGATGGGGAGTTCTGGAGGCTCAAGAGCATAGTGAC
AGCCTTGCCTGGGCTCTAGTTAGGGCTTCGTGGGAGATGGTGTCACAGTAGTAGAAGTGTGTGCAAAGAGACCACATGG
GAAGGCCCATTGGCTCATCTAGTCCTTCCTTTCAAAAAGCCAAAGAGATGAGAGATGGCTCCACGTTAAAAAAAAAAAA
TTAAAACCAGGAATGGTGACATACCCTTGTTATCCCAGTATTGGGAGATAGAGACAGGAGACTCCCTAGAGATCATAGC
CAGGCCAGCCCATTTAGTGAGTCTTGTTGCCCGTGAGAGACCCTGCCTCAAAAAAATAAGGTGGACAGCACCTTGGGAG
CATCCCCACGGGCAACACATGCTTAGGCACATATGCACACCCGCCTCACACCCACAAAAGAGTCCACCAAACTGGAGTC
TCTGTATCTAAGCGTCATAAACTGACTCAGTAAGAGGAGCCTTAACACTTGGCAAGTTAGAAACTTAGAAAGCCTGAGT
ATGAGTTCTTTCAGCGAACAGCATTGCAGAGGGCCCAGGAAAGCCTGCCTCCATGGTCTGGGCTTTGTGTTCTCAAAGG
TGATGGACAGCATGATGAAAATAGATCATCGCTTCTAGTTCAAGACCAAGAGAAGGCCAGTTGCCTTCCTCCACCCCCC
ACCCCCCAAACATCCTGAACAAAATTCTACATAACACTATAGAGCAGAAGGCTTCTTCCCAGCTCTTCCCAGTCCCACC
GAATGGCTACCTTCTTGTTTCTCTTTCTGTTTCCATTTGCACTTTGAGGCACTCTCGTTAATTGATGCCGTATCCACAA
CAGTTGGATGGGGTAGCTATGGACTTGTTCAAAGTAATAAATCATTTGCATTGTTCTTCAATTTGATCATAAGCTTAGT
TCCTCATGCTTTAAGAATCTGTGTCTCACTGGGAATTGTGGCATGTGCCTACAAACCTGGCATTTGGGAGGGAGAGGCA
GGGGGATTCCAAGTTCAAGGTCATCCTTGGCGACACGGAGACTTAGAGGCCAGCCTGGACTATCTGAGACCTTATCTTA
CAAACAAAAACAGAAGAAAACAAAACCCGAGTGTGGGCATCTACTCACATTCTTGCCTGGAGCCCCACGAATGCTCAGG
AGAAGCCCAGACAGTCATACCTTGGTTACGGCGAAGCAGATGTCAACACAGGGACCAGTGGGAAGTAGGCCACAGTTCA
TGAAAACATTCCAAGGTTCAGAGCCTTGCTCTGTAAACAGACCCAGACACCCTTGCTCTCTCCAGTGGTCTGCCTTGAT
CTGGGCGGTGTCAGTGGTGGTGTGTGCATCTCAGGGCTGTAGCCCAGAAGAGGAGGAACCTGCATCTCTCAGTCCTCAT
CCCCCCAATTCAGGAGATTAGCTTTCCCAGGGACTGCTCTTTGGATGTTTCTAGCGGTTCCTAGATGCCACGCAAATGG
CACGGGTGTCTTTTCCATCCAGTGTCCTTGTGAGGACCCCTCAGCTGCTGACTTTGGCTAGGCCTTCTCAAATACTGCT
CCAGGGCTCAGTGTGAGGCCTGGTCTCCTTGGATGTAGTGCCAAGGCTCGTGCAGTCTCTCCCAGAGAGCCCCTGGAAA
AGACCCATTGTGCTGGCTTCCAACCAGAACCCAGACATCTCCCAAGGACAGGCTCCTCTTCCCACACTCTTTGTCTCGA
CAGCCCTGTTCACCACGACCAATCTTCTGGAGCCTCCGACAACACTCCGGAGTCTACAGAGGACAAACGTCCCCTCCCG
AAGCAGCAGGGATATGCTGAATGCTATCTTTCTTTTTTATTTTTATTTATTTATTTTTTTTGGTTTTTCGAGACAGGGT
TTCTCTGTATAGCCCCGGCTGTCCTGGAACTCACTTTGTAGACCAGGCTGGCCTCGAACTCAGAAATCCGCCTGCCTCT
GCCTCCCAAGTGCTGGGATTAAAGGCGTGCGCCACCATGCCCGGCTGAATGCTATCTTTCAAACCAAACACTTTCTTTG
TCGAACATGAGACTCTGGAAAGAATCATTCTTCCTGAAAAGAAATGGGTTAGCTCTCAATATGTCTCCTTTGGACCTGT
CTAATCCCACATGGTTCCGTCAATGTACAAGATACTGCTACACTGTACCGATTACGTGCACAGCGAGGGGGCCGTGATG
ATGCAGTGCCCGTTTTCTCTGTAACCTCGCTTTGAAGGCTGCCTGCAGGATTTTCAGGAAAATAAATTGAAGACTAACA
TTTGGATCATGGTTATCTTAAATAAAAACGTATCCAAGCACTCTTGGACTGAAGAGAGAGAGAGAGAGAGAGAGAGAGA
GAGAGAGAGAGAGAGAGAGAGAGAGAGAGAACAGACAAACCAGCAAGATATCTCAGCAAGTAAAGATGCTTGCTGCCAA
GCCAGACCTGCATTTGATCCCTGGGGTCTGCGTGGTGAAAGGAGAGAACTGGCTCCAGCAAGTTGACCTCTGACTGCCA
CATGTTGGCTCTGGCACACACACACACACACACACACACACACACACACCAACAACAACAACAACCGTGTTCACAGAGG
AGAGAGAGAGAGAAAGACTCTGACAAGGGCAATCTACAGGAACGTCTTGAAGACAGACCAGGTCACACTGTGCCACCTT
GGCTCCCTAAAACCTGGCCTCTTTTAGCCAGTTGAAACAGTAGCCAAGAAAATAAATTGCTCTGCAATTTCTAATGGAC
AGGGTTTTCAGGTGACATCTGGAGGGAGGTCCTGGAGACAGTAATGGAAGTGGGTTAAAGGGCATAAATCATGAGTGTG
TGTGTGTGTGTGTGTGTGTGTGTGTGTGTGTATCCCTCCCCCTTGATTTTTTTTCTCTCTCTCTGTGTCTCTGTCTCTA
TCTCTCTGTCTCTCTCTCTCTCTCTCTCTCTCTCTCACACACACAACACACACTACACACCATTTAAAGAAGAAAGGAA
AAGAGCCCCTTCCCACAACTCGCTGGGGCACATTCCCACTCCCATCACTATTCTCTCAAACACTATCCCTGCTGTCACT
TCTGTCAGAGTTAGGCACTGTCAAGGACACAGTCAACACCTACCAGATGTCACACACATCTGGTCAGGAAGGAGACCTT
GAAGCTAACCACTGAGGCCTTCCTCAAAAGGGCATAGCCCTGCTTGCATGTACCCCAGAGACCTTGTAGAATGCACGAG
CTCAACCCTCTGGCTTTACAGGTGGAGAAACAAACTGACCCAAGGTTAGCCTTCAAGTCAACAACAGGGCACAGGCCTG
TGGCACGTTCCATCACAGAGAAACTGTGTGTTCGGCTCCCAGGAGACAAGCCCACACATCCATGATGAATGGGAAGGAA
CTTCCAGGGCCTTTTTATCAGTCTGAAGGAGGGACAAAGTGTGGGCAGGAGACCTGGGACAGTCATCAACAGAGTGGCT
AGCTTCAGACATGTTTGTGGCAGAAGGAAGCTCGGGAGAGGCGGAAAGGCCAGAGGCAATGGGGATTCTCCAGCCCAGG
ACATGGTGGCATCTGCTCCCTCCCTGATGCGGCACCTGCATTCACTCTTACGTACTGTGCTTGCCCTTCTCCCTGTCTC
GTCTCCTGGGTCTCCTTCTGAGATCACGGAGGGGAAGAAAGCAGATGACCAGGAGGTCAAAGACATCGAGACGCCCCAG
GGAGAAAGTGCCACGGTGCCAGCAACGCTGATGGAGGACCTCAGAGCTGTGTCCTCAGCCAGCTTCAGCGGCACAGGGG
CATGGCCTAGAGAAAATACAAATATAAATATTTGGGCACTAGAGAGGTGGCTCAGTGGTTAAGAGCACTGCCTCCTCTC
CCAGAGGTCCTGAGTTCAAATCCCAGCACCCACATGGCAACTCACAAGTGTCTGTAATTCCAGTTCCAGAGGGATCCAA
TGCCGCCTTCTGACCTCCACAGGCACTGCACACACGTGGGTACATAGATACACGCAGACTGAATGCCCATACACATAAA
AAAATTTAAAAATGCTTTTAAAGTGGTTAAAGGTGATAGTTCCTTTAAGAATTTTTCTCTAGGGCTGGAGAGTTGGCTC
AGTGGTTAAGAGCACTGACTGCTCTCCCAGAGGTCCTGAGTTCAAATCCCAGCAACCACATGGTGGCTCACAACCATCT
GTAATAAGGTCTGATGCCCTCTTCTGATGTGTCTGAAAATAGCTACAGCATACTTACGAATAATAAATAAATAAGTCTT
AAAATTTTTTTTATTCTTTAAATAGACACATGGAGCCACACCACAGACATTTTTTCTCAGTTGCAGATTGTGTGTCAGA
ATTTTAAACACCCTGTTGATTCTAATATAACCAAGGTTGAGATTTCTGTCTCAGAAGAAACTATGGTTTACTGTATACT
AGGTTTTTTACTTCTAAAACAAAAAAGAATCTAACCTAGGGTGATGGTGTGTTCCTGGACTACCTATGAGCCATCCCAA
TAGCAAATTTTAATTAATAGATGCAAATAAACAACAAAAGGCAATGTTTAGGGTTTTGTTTGGTTGGTTTTTTTTTTCT
TTTTTTTGAGGCTATCAAATTTTTTTTCATGTTCTTCATGCCTGCACCCCACAAACCCCCTGGTCCCACCACAGAGATA
TCAGGCTCCCTGTTATAATCACTTCCCTAGTATCCTAAGCCCCCGCTCCCACTGCCACTCTCTGAGCTGCAGACCTCAG
ATTCCCCTGTGCCCTTTCTGGTTCCCATGTCATTTGCTGTGGAGTTTTTTGGTTTTTTTTTTAAGATTTACTTATTTAT
TTTATGTGTATGAGTACTCTGTAGCAGTACAGATGGTTGTGAGCCATCATGTGATTGTTGAAAATTACAATCAGGACCT
CTGCTTGCTCCAGCCCAAAGACTGACTTACTATTATATGTAAGTACATTGTAGTTGTCTTTAGACACACCAGAAGAGGG
CATCAAATCTCATTACCAATGGTTGTGAGCCACCATGTGGTTGCTGGGATTTGAACTCAGGACCTCTAGAAGAGCAGTC
AGTGCTCTTAACCACTGAGCCATCTCTCCAGACCTGCTGTGGAGTTGAAACGTTACAAAGAACAGTTGAAACATCATCA
CCACTGCTATAAATGGGGCTGGAGAGATGTCTGGATAGTTGAAATCACTTGCTGCTCTCAGAGACCCAGGTTCTGTTCT
CAGGACCCACATGGTGGCTTACAACCACCTGTAACTCCAGCCATAAGATAACAGATACCCCACGGTGGCCTCCACTGGC
ACTGCACGCACATGGTAGAATACACACACAGGCAAACATTCATACACAGGAAATAAAAACAAATTTTTAAACCTGGTCA
TCAAGTCCTTCTTCCCCTACTGGGATGGTGATGCTAAGCTTGGTCTCCTGCTATACATAATGAGGGAATTAGATAAAGA
TGAGAAACCCATGCCTGGATACTGCACGTGTGGTGCAGAACTGTGATGAAGGAGAGTTGGGAAATGAACAGATACCCAG
GCTCACCCTGGCTCTCTGCAGGAAGCCTCTGTCACAGTACAGAAGAAAGGACAGGCAGAAGATTATGGTCTTGCTGGGT
TAAGCAGGCAGAGACTGGGCCTAGAGGAGTCTCCTTAAGGATGTAACTAAAAGCATACTGTGAAAGGGTAGCGTAAAAC
CCCACAAGGCCATGCAAAAGAACAAGGACTCTGCCTTCGGCACAATCCCCATCGCTTACACAGGCGGTGAGGGAGTGGA
GATGCAACCAGAGATTAGAGATGTCAACCACTGAACTCAACGAAACCCTAGAAAAGGCTTAATTTTTGTGCAAGGACTG
AGTAAAACACACCGTTGTTGTTTGGATGAGATGTCTCCCACAAATCTCGGGTATTTGAATACTTGGTCCCAGTGCGTAG
CATGATTGGGGAGACTTACGAGGTTTGGCCTTGCTGAAGGGAGTGTGTCCCTGAGGGACAGGCCTTGACGGTTCAAAAG
CCACGCACTGTTCCTCAGTGCTCCTAGTGAGTGAGAGCTCTCTACTTGCAGTTTGTGGCTCAAGATAGGAGCTCTCAGC
TCTGTGCTTGCTACCATGCCTTTGCTTGGCTGTCATGGACTGTAACCCTCCAGAACTGTAGGTCCAAAACAAACCCTTC
CTTCTATAAATGACTTGGTCATAGGGTTTATCACAGCAATGGGAAAGTAACTATACCCAGACTTAAAGCAAAGCCTCTC
TAGGTAAAGCCTCAAAGTAAACTTTAAGAAAACTGAGCTCACTCTGAAATTCTAAATGGGGATGTAAAAACAAAATTCA
ATATTCTCAAAAGATAAAAGCAAAAATCATTGCCTTTATTTCTTTTCTATTGCTATAATAAGACACCGTGACCAAGCCA
GGTGGTGGTGGCACATTGATTTAATCCCAGCACTTGGGAAGCAGAGGATGGTGGATCTCTGTGAGTTAGAGGACAGCCT
CATCTACAAAGTGAGATCCAGGACAGCCAGGGATACACAGAGAAACACTGTCTTGGGGGAGGAGGGACTCAGCAAGAAC
TTAAGAAGGGTGGACAAGATTGTGCAGTGCGTAGAGATACTTGCTGCCAAGAGGACAGCCCTGAGTTACATGAAGGAAC
ACATGGTTGAAGGAGAAAAGCAACTCCTCCGACCTCTACATACATGTTGCAGCATGTATATGTGCACATATGCACACAC
ATGCAAATAAATAAATAGATAAATGGAACTTTCAAATTAAGAAAAAAAATCAAACAAAAAAGAAAATAAATATGGGGAA
AATATTTGAAGAAACACCACCAAAGAAGATATACAAGTAATCACAACCTCTCGAAATGATGCCAAAGGTCCTTAGCCAC
TAGGAAATACAAAGTAAAAGTAAAGCCATAGCACATGTGCACACACACACTCAAACATACATTCACTCACACTCACAAA
CACACACACACACATACATTCACTCACACACTCACAAACACACACATACATTCACTCACACTCATTCACAAACACACAC
ATATAAACACTCATACATACACACTCACAAACACACACATATACACACTCACACATGCACATACACTCACTCATTCACA
CACACATACACACACATATACTCACACACTCAAACACATACATATATACACTCACACGTACTCACAAACACACACTCAC
ACTTACAAATACATAGTCACACAAACATACACACTCACACATGCACACTCACATACTCACACTTACAAACACGTATTCA
TACAAACACACACACACACTCACAAACACACACTCACAGACACACCCACACAGAGTCAGAATGGCTAAAATTTAAGAAC
TCACAGTACAAAGTGCTAGCAAAAATTTGGGACAACCAGAACCCTCATACACTGCTGATGGAAATGTTAAATAATAAAA
AAAAAAAAAACACTTGGGAAAACAACTGATACCTTTTGACAAGTTAAAAATACACTTATTCTAAAATTTAGGTGTCTCC
TCTTCAAGTATTTACACAAAAGAAATGAAAATGTAAACGCTTTGTGGTAGACAAATACCACAAACAACCCAAATGTCCA
TCACCAAATAAATACATAAGTTGATTGTGACATATCCAGTATGCTGGATACTACTTAGCAATTAAAAGTGCACACAACA
CACACACACACACACACACACACACACACACACACACATTCAGTTGTGTGTATGCATACGTGCTCACAAATGGACCATT
CTCAAGAAGCGTCATTCTGAGTAAAAAGAAACTCAGCACATATCTTACAATTCCATTTACATGAAACTCTAAAGATGGT
CCAGCAGCAGGGCTCAGTGGCAGAGCGCCTGCCAGGCGTTCCCAAAGTCTAGGCTTACTCCTTGGCACTTTAAATCAAC
AAGAAAAAAGTAACTCTGAAAGGGGCACGGAACCTAGGGTAGCAAATCGGATCAGTGTTTGTCTGCAGCCGAGAGCCAG
TAGCAGCGAACACAGAGACACTGATCTGGAATTTGTGGGAAACAATGCAAAATGAAAACACAGGGCTCCCTCCACACCC
ATCATCCCAGCACCCCAGAGGCTGAGGCAGGAGGATCATGAGTTTGAAGCCAGTCTGCCAGGTTCTGTTTCAACAAACA
AGTGGTGGCAGAGCCCTGAAGCAAGCATGGGGCCTTTATACGCTTGCACCCTGGGAGACCAACCTGGGCTTAAGAGAAT
TTTCTTGTGGTAATAGAAATATTTTGCATCTTGATGTGCATGTGTCAAAACTTGTGCATGTGTCAAGAACAAGTTGGGA
TGTAGCTCAGTGGGTGGACTGCCTTCATGCATGGCATGCATAAAGCCCTTAGTTCAGTCACCTGCATGAGCCAGGCCTG
AAAATAGGAAGTCATTTCTTTCTTCCTTCCCTCTCCCTCTCCTCCCTCCCCCTCCCTTCTTTGTTTTTTGGAGTCAGAG
CCTCATGTCCCCCAGCAGGCACCTCTCCATTAATATCTCTTCATATGCCCTATGTAGAAGACCCTCTATGTAAAAGAGT
TCCACAGAACACTCTCTATGTGGACTCTTTACAGAGGAGACCCTCCGTGGGGGGTTTCCTCCATGTGGGAACTCTCTGT
GTGGAATACCCTGTGTCAGGGAGACCCTGTGTGGGGGAGATCTTCTGTGTGGGAGACCCTCTGCGTGGGAGACCCTTGT
ACCAATCCTTCTCCTGCATTTTTTTCATTACAGGGAGTGGTTGTAAGTAGATGGTCATGATGGCTCCCTCAAGATCAAT
GATCTGGGCTTTCATTTAAGGTCTCCCACTCCTGGTTGGGGGGGGGGGGAATCAATAGGAAACCATCTGTGCAAAGCCG
CTAGTAACAACGTAAGTCACTCACTCAGCGTTCCCTGCGTGCGCGAATTCCGCAGGATTCAGCCGCTCTTTCAATGGAC
CTGCTTCTTTTTTGCGGTGTGTCTTTCCTTACTTAGCCTTTCTCTGGTTACTCTGCAGCTCGGAGCCACTCAGCTCTTC
TCATTTTCCTCGTTTTCTCCAGCTGTGTCTCTCTTACCTGGTGAAGTCTCTGGGTGGCTCGGGTGCTCCCATGGTCCTG
GCTGCTCTGCTGTTGCTTGTGAAGTTCAGTGACTAAGGCAAGCAGAATGGGAAAGGCAGCGGCGAGAAAACAGACTGCC
ACTGGGCTTCCTTCCTACCTCAGTCTTCCCGAGAGGACTATCGCACGGGCGTCCTTCAGATTGAGACCCTCTGAAGGAG
AGAATCCGCACCCCGGGGACAGCAAATCACACCGCAGACTTCCAAGACACATTTAAAATGAAGAGCCTTGTGGTTGGAC
TTAACTGAACAATATGTTGCCCAAATATGCGTTTTACTGAAGGCTGAGGCTATAGCTCAGTTGGTAAGGGTACTTGCCT
ATCACCTAGTCCCCAAAAGCATAGGTGTGTGTGTGTATGGGGTGGGGTGGGGGCCTTTTGAGCATAATACTCAGAAGGC
AGAGACAAGAAGATCTCTGCAAGATCTCTACAAGTTTATGGCCAGACGAGCCAGGGTTAAAAAGCTAGACAAAGCAAAG
TAAAAAGAACAAATAGCAATTGTTTTCACTCTTAGCACCACCTCAAAGTGAATGTTCACTCTGCCTGTGAGAGTAGCTG
TCTTAAGGGCTTCTCTGTTAGTTTCAAATCCAGGGTTAGATGATATCAACTGTGCATGCGCAAGGCTGCTCCTCTGTCC
CCAGAGTGGGCACTCAGGAACCCGCAGTCTTTCTTTCCCTCTGCAACACCTAACCATCCGCGCTAATCATCCACCCACC
CGGTGGCTCACCCAGAACTCCTCATCATTTGTCTCTGAAGGCATCGAACACAGAACTCAGGGACCAACTAAGTCTCAGG
TCCCAGGACTGTGAGTTTCAGGAAGACAAAATTCACAAAACCACTGTGGAGTCGGGGCAGTACTCTAATGTCACATGTA
ACCAAGTTCAAATGTTAAACACAGAGACTGAGAGAGACAGAGAGGGCTGGAGAGATGGCTCAGCCGTTAAAGGCTAGGC
TCACGAGGGAAAATGTAAGAGACAAAGAGCTTCAGGGGCACCTCCTGTCAGCAGATGGAACCGGGTGAGGGGTTCAGAA
GTGGCTCGGAGCTGTTTTCCCTTAGGGCACAGCAGAGAAGAGAAGAGATGTACGTCTGCCTTGGCCCTTCCCGGTCTGC
TTTTCTTTTCTGTTTCCCAAGCTACTCTGCCTTTAAGAGATGCAGGCAAAGCTTGAACGGCAGCTACAAACAGAGATGA
GACACTGGAAACCAATCTTGGGTTTCACCTGAACAAATCCCTCTCACAGCAACCCGACACCTGCCCAACTGTCAGCTTT
CCACGATCCCTAATTGGTCTTCACAGAACCCGGCCACCTGCCACCATCTGCCCTGGACCACCCTCACACCTGCCCCTCC
AGATGTTCACCTTCTCTTGGAAAGCTTCTGGTCCCCAGGCCCTCCTCCCACTTCTTTAATCTTCCCTGACCTCCAGAGT
TCTGGGGCATGAAGCACAGAGTTAGAGGCAGCTGGGTCCTGGGGAGAAAGATGATGACCACCACAGCCTCCTCTTCGAA
GTGGTCAGCAGATTTCTGGCTGTGTTATCGAGAAAGTTCCTAGAGGCCCCTCCCAAGGTATCAGCTCATCTCCTCCATG
TGAACCTCCAGGCAAGTGGGGCTCACCCCACCCCACTCCCGTTTTCCACTAAGCCTCCTGTGATTAAGCACACAAGATT
CCTCCCATTTTCCACAGGTTATATCAGGCGGCAAGAAAGCTTTAGTTAGGCTGGACAGACGTGGTCACATTTGGACTCT
TTATTTCACGAAGCAGGGCCATTTGGATGTTCCTGAACCCTCATTTTTACAACAATTTAAATAATAAATTTGAGAATTT
CATACAATCTTAAAAGCATATTCACTCCACCTCCCCCATCTCTTCCCAGATCCAACGTCATCATCCTGCCCACTTAACT
TCGTGTCTTCTTTTTTTAAAACCCACCAAACCCTGTTTGTACTGCCCTAATGCTACCCTGGGTGTGGTTGGCCCATCAA
GGGGTCACAACCTTAAAGAAAACTAGCTCTCCCTCTTCCAACAGCCATCAGTTGTCAAGGGCTCCTCAGCTAGGGGCGG
GACTTCCTGCCCATCTCAGCTCTCCCTGCTGGGATTTTGTCTGGCTTGATCTTGCGCAGGTCTTTCAGAGGTTAACATG
GCACTTGAGTTCTTACGTGGAACTGCCCCGTTGACGCCACAAAACACTGTTTCGTTGTAGCCGTCTGCGGCGGCTGGCT
CTTACAATCTTTCTGCCTCCTCTTCTGTGATCCTTGAACCTCGGGGAGAGAGGACAACACCTTTTGTTTTTGTTTGTTT
TCTTCTTTCTTTCTTTCTTTCTTTCTTTCTTTTCCTAAGATTTATTTATTTTATGTATACATGTGCACTGTAGCTATCT
TCAGACACACCAGAAGAGGACATCGGATCCCCAATACAGATGGTTGTGAGTCACCATGTAGTTGCTGGGAATTGAACTC
AGGACCTCTGGAAGAGCAGTCAGTGCTCTTAACCTCTGAGCCGGCTCTCCAGTCCATTTGTTTGGTTTTTTTCAATGAC
TCACAATCGCTTGTATTGATGGAGTTCAGAATGACATTTTGATACTTGTTAACGAGGTGCAATGATCAGATCAGGGTAA
GCCTCCTGTCCAAGATGCTATCTTGGAAGGCCTCAGGCAGCTTCCCCGACCTTCCAGCCAGCTTGGTCCCTCTTCGGGT
TCCCACTGCATTCCACACCCCTGCCTCTGGCCTCTGACCATAACCAACAGAGAACTCAGCCCCATCTTTAATCACCTCA
AACACCTGGGATGCTAAGTGCTAGGGGCAAGTTCTGGGGGTAACATACTCTCACTGGGGCAGCCACTAGAAAATTTAAA
TATGGCTGTCTGTCTGAAAGAGGCTTTATATGGGATGGGAGATGTCTCAGTGGGTAAAAGTGTTTCAACCTGATGGCCT
GAGTTCAGATCACTGAAACACACATTAAAAAACAAAAATAAAAACAGATGCAGTACTACAAAGCTGTAGTTCTAGTGCT
TTTACAATGAGATGGGGGAGAGGGGAGAGACAGGAGAATCACCTGGAAGCTACTCATTGGCCAGCTAGCCTAAAGTATA
TACATATACTTAAATACTAAAGAAGCAGAAACAAGAGACACCTGAACTAAAACAAGGTGGATGGCAAGAACCAACTCCC
AAAAGTTGTTCTCTGGCTTCTTATGTGCTGTAGTCTCTCTCTCTCTCTCTCTCTCTCTCTCTCTCACACACACACACAC
ACACACACACACACACACACACACACACACAATTTATGCCCTTTAACCCACTCACATTACTGTCTCCAGGACCTCCCTC
CAGATGTCACCTGAAAACCCTGTCCATTAGAAATCACAGAGCAATTTATTTTCTTGGCTACTGTTTCAACTGGCTAAAA
GAGGGCAGGTTTAGGGAGCCAAGGTTGCACTGTGTGACCTGGTCTGTCTTCAAGATGTCCCTGTAGATTGCCCCTGTCA
GATTCAGGCAGAGTATGATGAAGAAGTGTGTCTATATGTCTGTGTCCTGTAGGCTCCCCAACTGCCATCCCGATCATAA
AAACCAGGGCCCATGATGGTCATACCCTGCACGAAGTGGCTAAAGCTTAGTCTTCATGTCACCATAGGTGTTAGGGTCA
GAGTCCGGATCCTTACAGCCCTAACATGTTAGGCTCAGCTACCAGTCCTCAACAGGTCAATTAAACAAAGACCAGTGAA
AAGCAGAGAGAGGAGATTTATTCATTATGACCATATTGGAAAGAGGAACAATGAAATCTAGTGACTGTCCATCCACTAA
ATTGCACCACCACCATCACCACCGCCACCACCACCATCACCACCACCATCATCACCACCACTACCACTACCACCACCAC
CACCACCATACCCCTCCTTGACAGGCCATCTTCCCAGGGACCCCATAGGATGCATAACTAAGCAGTCTTAGTCAAGGTA
GAGTCCAGCCTTTCACTATCTCCTTGCAAAGGGTCTGAGCTATGTGACATCTTTTTCAGGAGACAAGTCCTCCCGACCT
CCTTCAGCTGGCACTAGCTGTTTCCTTCCCCCAACATGCAAGTTCTGGGGAATTCTATTATTGGGGGTCTGTTAGCTGA
GTGAAATATCTTCATTTATGTCAGGACTGTTACATTATGGGCCAGGCCATGTTCTAAGTATCTGATGTGGGGGGGTGGG
GGGGAGTTCCCATGGTTTTACAGATAGCAGACCTGAAAGACAGAAAAACTAAGTTGTTTCCCCACACAGCAAGCATCTG
AGGAAGCTGGCTTGGGAGGCCCTCGCGCACCCATTGCAGTATTCCTATCCTGGCTCTTGCTCGCTCCAGGCGTGGTCTC
CCATATTCTTGATCTGAGCAGGATCAGTGTCTATGAAAGCAGAGCATGGGGTTCTGTGTTGTTTTAATCCCAGCAGCCT
TCCTGCTACCCAAATAAGACTCCTGGGAAGGTAGTGGGGAGCCCTGATGGTGCTACAGCTCTTGGCCCCTTGCTCCCTG
ATGCCGAGCTCCACTTACCTCTGAGAAAGGGCAATAGGCTCCATTCAGCTCCTAAGATCAGACTCCCTAGCAACAAAGG
CCAACTGATCAACCCTGCTGGCAGATGGGGCCTCTTCATTGAGAAATGAAGAAGGCCATCTCTCTAGGGCAGTTTAAAC
ATGTCTTTCACTGTGCTAATCATTTTACAAACACTGTCTAATCCCTCAAACAACTAAGGGCAGCTCGCTGCAATCGTAC
TGTTGTGACAGATGTGCAAACGAGTTTCAGAGTCGTTAATCAATTTGTCGTAGACCACACAGTTAGTGACGGATCCAGA
GTGAAAGTTCAAGTTTATCTTTCTACAAAGCCCTAAGTATCTGTTTGACAATTTGACTATGTCCTTGGGGGGTTGTGTT
CCCCTGCCTACTGTGAGACATCACTAGGGAGAAACTTCTGCTTCTGGGCTGATGGCTTAGCAGGTTCAGGGCCTGTTCT
GAAACAGTCCATTGAAAGGGCAAACATCTCTCTCCACCCATATAACCTACTGACACTCCTACCTATGACTTAGTACAGG
TACTCAGTACCTTAGGACCTATTTACACTAGGAACAGAGGCACATCTCCATTGTTATGGTAGGATGGAATAAGGCAACC
CTTGGCCATCAGTCCTAATCAGAACGCTCTGGTCCTCAGCCTCCCAACTCATGATCTGGCCGATAGTTGGGACATTGGA
ACCCAAATGCTCCACAAACTAAGCCTCACCACTTGACAGGACATTACCAGAGTTCTTAGGGTCACACCCTGGGCCCAAA
AGCCCTAACAAGCTAGGCTCGGCCACCTGCCCCCAACATGCCAATGAACGAAACATGTAATCATGAAAAAGAGAGAAAA
GAGATTTCTTTTATGTGGCCACAGTAGAAAAAGAGACAAAAAGGAGGCTACAGGACCCGCCAATCTGCAGTTGGAGGTC
CTAACACAAGCTTTGGGTTTAAATAGACAATAAGGGGTCACACCCATCACTGACCCATCATTGTCCTGGCTCTGGTCTG
TCCAGAAAGGTGGTCTGAGAACTTATCAGAACTGAGTAAAGCTTCTTCCTGTGAGACAGGTTCTTCCCCCCCTCCCCCT
CCCCCTCCCCCTCCCCTTCCCCTTCTCCTTCCCACAACATGGGGCTCCTGGAGACAAGTTCCCTCTTTGAGGGCCTTGC
TTCAACAGTCTGATAGCAGAAAGGAGGGACACCAGCGTCTGTCGTTAGAATCTCTACTCCTGCCAGGAAGGTCACACCA
GGACCTGGGGCTGCAACCTCCTTCCCTAAGATCTAGTCTCAAAGCAAAGCCCTGGCACCCCAGTGCACCCCCTGTCCTT
GCTTCCTTCTGCTCCCCTCTGTTGGAACTCTTCCCAACCCTCAATTAATTCATAGATTGTAGCAAATTGCAAGAATGGG
TGTGGTGGCCCATATCTTTGATCCCAGCTCTTAGGAGACAGAGGTAGGTCGAACTCTGTGAATTCACTATTATCCTGGT
CTACATAAGTTCCAGGTCAGCTAGAGCTACCTAGTGAGACCCTGGCACAGAAAACAAAGTCTGGTTTGCTCGCTGTTTT
AGAGTCCTCCAGCCCTAGACTATAGGAAGTATAAGAGTGGGAGGCGGGGTGGTGAGGGATGGGGGGATGGTGTGTTTTT
GGAATTGCTGGGGGCTAGGACACAAGACCTGATACACAGTAGTTACTCCATAAATGCTGGAAAGATGCCTGCGGACTGG
ACAGAAGGGAGACTTAGAAGGCTTATGACCCTTTAATATCCCGCGGGAGTTTGGCTTTCGAGCTGTGCTGTGCCTGGGA
ACTAAAGCACACAGCAGCCGTCTTCACTGTAGAAATACGAACTGGAAAGCTGTTGTTCCATTGAGGTTATTTTGGGAAG
GAAGCACAGTAAGACAAAGCCACCTCCAGGAATGTGAAGGCCTCTTTCTGTGGGCCCCTGACTCACCTTCCAGACAGAG
GACTGACGTTGCTCTTAGCCAAGGTGTTCATCATTCCCAGCCATGACTCCCCTGGCCTTGGATGCTTGGGATGACTCAA
GGCACACTCCATCCAGACCTCCTGCAGTGGCCTGTGGCCATGCCAGCTGGGCCCCAAAGCCCAGATGCTGCAGCCGTCA
AGGGCTGAGTTTACTGTCTCCATGAATGTCTAAGTCCTGACTCTGCTCTCAGTCACTGGGGGGCGTTTTCCTCCTTCAC
CGAAGACGGATAATTTGTCACTTATTTTTAATTGCTTCGTTTGTTTGTTTTGAGAAAAAGTCTGGAAGAGCTGCCCAGG
CTTGCATCAAATTCCTCAGTCGATTCTCCAGCCTCAGTCTCCCAGGCAGTTGGAAGTAGGTGTGTATCACTGTATCTAG
CCGCTCCCGGTCTCTTTCTGAAAGGGTCTCATGTATCCCAGGATGGCCTCCACCTTGCTATGTAACCAAAGGGAACCTT
GAATTTCTGACCTACCTGCTTCTACATTCCGAGTGCTGGGATCATTGGTCTCTACCACCACATCTGGTTTATCTAGTGC
TGGGTATCAAACCCTAGACTTCATGAGTGCTAAACAAGCACTCTTGGCCCTTTCTTGAAGGAAGCAACTACAGCTCAGA
GAGACCAGACGCAGGAGGGCTAATCATGTGTGCTCTGTCTCCTTTTTTTTTTTTTTTATTATTTTTTTCCTGATTCTGA
GAACCAGGCAGGCCAACCTGTCACTATGAGACAGCCCGAACCTTCCTGAGTGTCACTGGCCAGGAACTCTTGAGAAAAC
TTTGGGAGAGATCAGAAAGACCAGGATGAGGGAATATCTGCTTGCTCCGCACTAAAGGTGGCTTGCTGTAGCTGTGTCC
AGAAGGCTCTGCCTGGTTAACGCCAGCTTTTGTGGTCCAGCTGAAGTGGCTGATGCTGCCCAGGGTAAGCAGGGATGTG
CCCATAGGTGTTGGGTCAGAGAGAAACCACTATTCTTCATATTCCAGGATGGCATCTTCTGATCCTGTCCTATTTTGCT
CAAATTTGACTCTAGAAACTCACAGCCTCCTCTCCTAAGAGATGCCTACCTGACAAGGTCATGTGGCCCATCAGGGTGG
GCAGCCAGCTTCAGGCTAGGCATGTCCCCACATGTCCCTATAACCATCTTCAGCTCACAGTCTCATTTATCAAAGTACT
ATTATCCTCAGAACATCACACAGGGCTGACTAGGTCCCTGGAAAATAACCACAGGGATGCTAAGGCAGGCCAGCCTCTC
ATTTACAATGGGGAAAACTGAGACCTGGTCTATATGGGCATCGAAAGAGCAAGCTTAAGCTAAAAGCGGGTCTTGTAAT
CCCAAGATGGGAGGTCTCCCTATGAGCTATCTGAGTGGCCTTGAGTGACACCATTCAACTTCGCTCAGTGGGCACTTAT
GACACATGTTATCTGACTTGTCCTGGGAAAGTATGAAGAAAGATTTGTTTGTACCATGGCAGATTAAAGAAACCATTTC
ACCCTTAGTCTAACTTAGTGAACCAATAAGAGTGTATTGGGGTTCCTTGGAGAAGTGTGGGTGAGGGGTTTCTTTCGGG
GATATGAATGACTCAGATGCATCTGTTGAAAAGCTCTCCCCAGCACGGTGATGACATGGGAAGGCTGCATGCCTAGAGC
TTTCTGCACAGCTTGCAAGGCAGCCCTGCCTAGCAGGGGATCTCCCCTCTTCTCCAGCAGTTTTTCCTGCGTATGTAAC
CTTGGGGTGAGAACCTGTGGCTCTCCTAAGTTTCTTTGTGTCCTGGTTCTTGTGAGCCTTGCTCTTTCCCCCTAGAGGG
GATGTTTCCACTGGCAGGAGGCAGCTATGTGCACCAATACCTGCACCAGAGTGGGGGTAAGATGGGATTTGGGAGGGAA
AGGAGGAGAGAGCGGAGGAGATTTTGTCATGGTGGGATGGACGGCTGTGTAGACGAGAGAAGCTCCCCTGAAACTCCCC
ACAGGAGCTGATCGGCACATTCCCAACCTTCAACCCTTAACAGATGAGGCAAACAACTTGGGCTCAACAAAGCAGCTGG
ACTTCTTAACTCTGCTCTATTGACCTATCTCATAATCATCTATATATCACCCGTGTGCCTACTTACCACCTATCTAGCC
ATCCACCGTCTGTCTGTCCTCTGTCAATGGTTTGTGTTAACGTCCCTCCAGGTGGCTCTGCTCTCTCCTTCCCTCTGTC
CCTCGCTATCAGTCACACGACTTCACCAGACTGAATTCAGAACTGAGAATACCCCTTTGTTTTTCTGACTTTTAAAGTT
GCCATGTTTTCTTTCAAGTCCCCTTCTTTGTCATGTGGTTTCCTTTTCTGAAACTCTTCACTGTGGGCAGTCCTGGGGT
GGGTGGGTGTGTATCTTCAGCAAGAACCCTGGTGTCTAAGGGCCTGACTCCAGTTCCCTGAAAGGTATGAAAGGCTGTG
CCTGTGGTGGTTTGAATAGGTTTGATCCCTATAGACTCATGTACGTGAATGCTTAGCCCATAGTGGGTGGCACTATTAG
GAGGTGTGGCTTTGTTAGAATAGGTGTGGCCTTTGTTGGAGGAAGTGTATCACTGTGGGGGTGGACTTTGAGCTCTCTT
GTATGCTCGAGCTATGACCAGTGTGGCACACAGTCTCCTGCTGACTTTGGCAGCTCCTCCAGCACCATATCTGCCCGTA
TGCTTCTCGCTGCCTTGATGATAATGGACTAAACCTCCCAAACTGTGATCCAGCCCCAGTTAACTATTTTCCTTTATAT
GAGTTGTCATGGTTATGTCGTCTCTTCACAGCAATAAGACCCTAAGACAGCGCCCATGAGGGAAAGGCATCTTCTTTAC
TCGCCTCACCTCAGGGTGAACTCTCAGATGTCATCTCAAGAAGCCCAGGTCTCCTTATCTACACCACTTTGATAAACTT
TGGCATTCCCTGCAGGCCCGGGATCCAGTTGCCTGGCTTGTGCTCTAAAGGTCCATGGTGCTGAGCTGATGGCCTCACT
GCGGCTGCAATCTGTCACTCACCACCCTACTAGGGAGCAGGTATTTCTCTAGGCTTGATGTGTGGATAGCCATTACTTT
TTAATTGGTGGACATATGGCAACCTTTATGGAGACAAACACCCAGACCAACCAGGTCTCATATCCAACACACAGAGACA
GATGTGAACACACATACATACATACATACATACATACATACATACACACACAGATGACACATAGCCAACTGTGTTCCAG
AGCAAAAAGAGCTTTTCAGTTGTTGTTGTTGTTGTTGTTGTTGTTGTTGTTGTTGTTGTTGTTAGCATTTTCTGTGTAC
GCTGTGGGAGGGAGGTGTAGCAGGCATGCTGTGGCTCACATGTGGAGATCAGGGGACAGCTTTGTGGTGTCAGTTCTGT
CTTTCCACCATGAGCTCTAGGGACAGAACTCAGGCCATCAGGTTTGTGCAGCGCGTGCAAATGCCTTTGCCTGATGAGC
CATGGCTGGGCTGGAGGTTGATGGGACCAGATGCGTAGGCTCCCCAGGACATATCTCAGCCCAAAACGGTACAGGGGAA
GAAACCAAGGCTGGAAAAGACAGCCTATATGAAGGAAGGAATGGATCTTTAGGTAGAGTCAAGGAGGTAGACATTTTCT
TATCAGAAGTGGAGGACAACCTCTTGTCAACCACCCCTCTCTCTGGCCTTCCTGTTCCATCTTTTGGGGAACTCCTTCT
GCAGCTAAGGAAGTTTAGCAGTGGCATTGAGAATGGAATTCACTGGTCTACCACATACTCCATCACTTGGAAGCATCCG
GCCAATGAGCTGTGGAAAGGCCTGCTGGGGACTTTGTTATGGCACTGAAAGGGAGACAATACCCAGAAGGGCAGGCTAT
GCGGTACAAGTTGTACTATAGGCTTTGACTCAGCAAATGATATGATATAGCCTATCTGTAGCCAGGAATCCCATAGCCC
TCAGCCCGGGTCTGGGAATGGAAAGCAGGAAGCAAGAATGACTCTTTCCCACATCAACTTAGCTTGTGCTGGCTTGGAG
GCCTTAATCCCCAAAAGAGGGACTATCCCACTTTTATCAGACTGAAACATCAGACCTCCATCTGGCCATTTGTGAATCT
TTGTGCTATTGAGACAGCAAGCACAATATAGATAAGGTTTGCTCCATAGGCAGGATGACTGGACCTAACAACCAAGGGA
AAGTCAGGGCCTTGCTACACAGTGGTAGCAAGTAGCACCATGTTTTGTCCACAGTGAAAATATAAACAGAAAAAAGTAA
GAGCTACTGAGATGAGCTGACCCAGGACAAAGGTTGAGTCAGCTCACCAGGTCAGGAGAGCTGCTGTCTGGGAACACTG
CATGAGTAAAAGGAGGAAGAAACAACAAATAGGTTCTGCTTCCAGAACTCTTTCAAAACCTCATGCCCAGCCATGCCTG
TGAAGACCAGAACAATAGCACCGTGTGTGTGTGTGTGTGTGTGTGTGTGTGTGTGCTGTGTGTGCGCGTGTGCTGTGTG
TGCGTGCACGTACATGTGTACGTCTGTATCTGTCTGTGTGTATACATATGTGTGTATTGTATGTCAATGTCTATATGTG
TGCATGTGTGTCTGTGTGCATGTGTGTGAATGTGTCTTAGTTAGAGTTTCCATTGCTGTGAACAGACATCATGACCAAG
GTAACTCTTATAAAGGACAACATTTAATTGGTGCCGGCTTACAGGTTCAGAGGTTCAGTCCACTATCATCAAGGTGGGA
ACATGGAAACTTCCAGGCAGACATGGTGCAGGAGGAACTGACAGTTCTACATCTTCATCTGAAGGCTACTAGCAGAATA
CTGACTTCCAGGCAGTTAGGGCAAGGTTCTTAAAACCCATACCCACAGTGACACACCTACTACAACAAGGTCACACCTT
CTAATAGTGCCACTCCCTGGGCTGAGCATATACAAACCATCACAGTATGTATGTGAAAGTGCATGTGTATGTATGTATG
TGTGTGCTCTTCTTCCCACTTCATTTTATGTAAGTTACCTATGAAATTTGTATTGTTTTTAGGTTATGAAATAATTATA
TGAGACTATGACTAAATTAGAGAAATAATTATTATCACCTATGGGTAAACATGGTGACTATGGCCAGGAATGTGTGTGT
GTGTGTGTGTGTGTGTGTGTGTGTGTGTATTCTTTTTATGTTAGTTATAATAGTGAGAAGGAATTTCACATTTTGATCT
TGATGACTGCTGAGATCAAGTTGCTCTCACAAAGTATAGCTGCACCTTGTTATGTCAAAGCAAAAACTTGCAATTAATA
TGTGAACACTGAAGTGTTGACAGAGGACTTATATTAATATTCAATATATATTGTTTGCATGAGGTACAGAAGGTATATG
GAATCCCAAAGAGACACGATCAGAAAAGTACCTCCCTGTGCCATACTGTAGTTAAAATGCCAAAAGTAGAAAACAGAGA
GAATACACAAAGCTTCAAGGGAGAAGTGAGAGCTCTCATACAAAGACAGACTGACAGACCAACAGACCCAGCAGAACAA
CATCAGATTTCTCAGCAGAAATCCTGAAGGCCGAGAAATTACAGATTGTCTCTCAAACCCTGAAAGAACATAACTATCA
GTCTAGTCTACCATATTTATCAACATTCTCCTTCCAGATCAAATTAGAAATAAAGACCTTCTAAGATTGGCTGGCATGC
TCTAAAGGTATGGAACATTAAGCCAGCCTTGAGGAAGACACTTAAGGGAATTCTGTGTCCAGAAGAGAAAGATAAACCC
AAGCACAAAAGGAAGAAAGGAATTTCACTACATGAGTAGCCAATCCAATGAAAATTAGGAAATCATCAAACAGTAAACT
AAATGACAGCAGTGCATGCCTTTAACCAGCATTTGGAAGTTAGAGGTGGGCAGATTTCTGAGTTCAAGGCTAGCCTTGT
CTACAGAGTGAGTTCCAGGACAGCCAGGGCTACACAGAGAAACATTGTCTCAAAAAGAAAGAAAGAAAGAAAGAAAGAA
AGAAAGAAAGAAAGAAAGAAGGGAGGGAGGGAGGGAGGGAGGGAGGGAGGGAGGGAGGGAGGGAGGGAGGGAGGGAGAG
AGAAGAGAAGAGAAGAGAAGAGAAGAGAAGAGAAGAGAAGAGAAGAGAAGAGAAGAGAAGAGAAGAGAAGAGAAGAGAA
GAGAAGAGAAGAGAAGAGAAGAGAAGAAAAGAAAAGAAAAGAAAAAAACTTACTGACCAAAATTTGTCCATACCTTTCA
ATTATATCCCTGAATGTAAATGATCTTACTTCTCCATTTAACAGACAGAGACTTGGTGGTTACATTAAAAAGATATTTG
TTTGCTACCTGCAAAATGTCTGCCTCATTCATTGGCAAAGATATACACAAAGGATGGAAATTAATATTCCAAGCGAGTG
TAATCACGAAGCAGGAAGGGACAGCTATGCTCAGGTATGACAAAGCTGAAGAAATTAAGCCACAACCAGTCGGAAGAGA
CAAAGAAGGTCACCATAAACTAATAAACAGAACAACAGCCCATCCAGAAGATGAAGTGGCTGTAAAAATGCACACACCG
AGTGTACAAAACAAACACTATGGAATATAAAGGGACATCTATGTCCCAATACTGGATAATAATGGGTGACTTCAGTAAC
GCACCAACACCAATACATAGAGCACCCGATCCCTCTCCCCAGAAACAGAGACACTTCAGAGTAACATTAAGCGACCCTA
CAGACCAAATGGACTTAACGGACAACTATCTACAGAACTCGTAGCTCTTCGGTTCAACAACTGCACATTCTTCTCAGTA
GCTCATGGAATTCTCTCCAAAGCAGACCCTATTTTAAGGCACAGTGTAAGACTCAAACAACGACTGAGGGTGAACTCAC
ACAGTATGTCTTATTAGATCATCGGAAGGCACTAGAAATCAAAGCAACAGAAACCAGCCAAGCACGTGGAGACTGAATG
GGCGATATTCCTTGTGAACACAAAGCTGTATAATCTTCTTCAATTACTCATCCTGGAATTACCATTCAGAATATTAATA
ATTAGATAAAAATAAAAGAACTAAACTTGGACAACAAAAGAACAAGTAACCCAAGCGACACGTGTAAAAATGAACTGAG
CACTTACCAAAGGAAATGAAAATGGCCAAGAAACACGTGGGAAAATTTGTATGTTTAATCGTCAGGGAAATGCAAATCA
AAATTGCGTTTGTCAGAATGGCTGCTATCAAAGGTGCAAGATGCTGATGAGGATATAGAGGTGAAGAAGCTTGTATGTA
GGGGCTGGAGAGATGGCTCAGGGGTTAAAAGCACTGACTTGTTCTTCCAGAGGTCCTGAGTTCAACTGCCAGCCACCAC
ATGGTGGCTCACAACCATCTGTAATGGGATCTGATGCCCTCCTCTGGTGTGTCTGAAGACAGCTACAGTGTACTCATAT
ACATAAAATAAATATTAAAAAATAACACTTAAAAAAAATAAGCTGCTGGTTGGAAAGTGAAATTAGTCCATTCACTATG
GGAACCAGAATAGGATTCCTCAAAGAGAGAGAGAGAAAAAGGGGGGGGAAAGAAAGAGAGAGAGAGAGAAAAGGAAGGA
AGGGAAAGGAGAGGAGAGGAGAGGAGAGGAGAGGAGAGGAGAGGAGAGGAGAGGAGAGGAGAGGAAGAGAGAGAGAGGG
AGAGAGAGAAAAGGAAGGAAGGGAGGGAGGGGAGGAAAGGGGAAGGGGAAGGGGAAGGGGAAGGGGAAAGGGAAGGGAA
GGGAAGGGGAGAAGAAAGAACTGCTTGACACTATTCATGATAGCCTGAGTGCCTGAACAAATAAATTATGGATTATTAT
TATGGGATATTATTTAAACATAAAGATCATTAAGGGAGAGGCTCAAGACTGGGATAAAGTTATCTGTGCTCTTACATTT
TTTTTAAAAAGGGTTGAAGTTTACAGTTGGGTTTGGGTCGAGTCAAAATGTCTCCACATCAAAACATCTGTAAGCCAAA
CAGCTGCTCCCAGTGCCGTCAAAATGGCTGCAGGAAAATTCCACAGACTTCCCCTGTAGGAGCCCCTCGCACAAACTCT
TATTTCTGTACTCTGCCAACTTGAGGTCAGTGTGGCTTAAAAAAAAGGGTGGTGCTGGAGTCAGCCAGCCCAGGCCTCC
CAGGCCAGCGCTAAGTAAACAGGAGTCCTGGGGGCGACCATTGCTCCACGGGGGCCCTGAGGTGGGATTTTCCAAAGAA
ATGAAAAGCTGCCTTGTGTGAAGAGCGAGTGTGGGCCCAGCATCTGCTTTCCATGACCTCATCTGTGTTGACTTTCTTG
CCATTTCATCTCTCTGGAGCCACAGAGAAGGCAGAGTGGAACCGGTGAACAGCAGCCTTCACCGGAGAAGGTCAGGGAA
GGAGGGATAGGTGACTAACTTCAACCTCTCAGTAGGCAGCTAGGGTCTCCAGGAGGAGACTCCTAGGCAAAGCCTAAGA
AGGGGTTGGGGGGAGCCCAGGTGTGTCCAGGTGTCAAGTGCTTCTATTGTTTTTCTCAGGAGGAACTCTGATGGAACTT
ATACTCTGTAATTTGCACACACTTCCTGGGGGAACCACAGATAGCCATCACTTTATAATTACCTGCCTCCACTCTTCCG
CTTGGCAGCTAGCTTCCTCCAGTTCTATTCTTATGATCCTTCCCAACCTAGAGAAACAGGACAAGATGCTTTAAATGGC
CTCCTGATGAGCCCTTTTCCCTTCCAAGGACCTTTTCCTACCCTGAGCTGTCTTGTGAACATCACCTTTAGCTCCTCCA
CACATGAGCTCATTCTTCTAAGAGAAGAAAGGAAGGGAGCAGAGTTATGTTCCAGTCAAGAATAGACCTGAATGTCATC
TCCTGACCCAGAGGAGCCAGCCTGGAGCAGTAGTTTAAGGAACACGGCATCATCAGCTGGCAGGAGGGACCACACCTTC
CCTGGAGCTAGTGTACCAGACAGAGAAACTAAGTTTGACCTGGTGCTGTGGCTGTGCCACCAGAGGTTGTAAGGTGGCA
CCTTACTGAGCATCAAACAACCTTGAAGAAGACAGTGAAACAGCTATTCCCTCTGCAAACATCTCTTGGTCCTTAAAAT
CATAAAGGGTGACAAAGCTTTCATACCAGGCGAAGGAGGGTTGAAAACATTTTTTAAATTATATTTATTTATTGTGGGT
GGATGGATGTCTACACATGCGTGTGGATGTCAGGTGACAACTTGTAGTAGTTGATTCTCTCCTTCTTCCATGTAGGTTC
CAGACATCAAATGCAGGTTGTCGATCTTGAAAGAAAGCCCATTTATCAACTGAGCCGACTCACCAGCACATTAAAATCT
TTTTCTAATTTCTAGTTATGTAGTGTATAATTCATTTTCTGTTTATGGTTAAAATTGGGTTTTTTTTGTTTATCTGTTT
ATTGTTCTCTTAGTTTTATGTTTTCTTTCTCAAATTGGAGGTGAAGGAGGAGGAAGAGGAGGAGGAGAGAGAAAAAATA
GGAAAGAGAGAGAAGGTAGGAAAGAAAGAAAAAGAAAGAGAGAGAGGGAGGGAGGGAAGGAAGGAAGGAAGGAAGGAAG
GAAGGAAGGAAGGAAGGAAGGAAGGAAGGAAGGACAAAGCCAGATGTTAAAGGCAAAAGTAGTTTTTAAGGAAACCCAT
TTGCCTAACACTTCCAGGTATGTAGAAAGACTGGGAGCAAGGACCCTCAGATACCTGCAGAGGCTGCTGGGTAGAAGAT
GAGAGAGTTAAGAGAGCACTTGATAAATGACTGCAACAGTTGCTTTGTTATCCATCTGTATACAAGCTAGAGCAGACCA
CACTCACATGGTCATTTCTGCTCCTGTTTGAGAAATGGGTGACGGACTTAGTCTCTTTTTAAAGTGATCACATATATGC
ATGGGCTCTCGAATTGTTTACCCCACAAAAATGGTATCAGGTTCTGACCCTGCTATCCTAAATTTTGCTTTATTCATTC
CAAAATTCTTCTGAGAAGCCCGGCACTTGTTTCTGAGAAAATACAGCACAATCAATATGAATACATGCTGTGCACTTAG
ATGGGGCAGATCTACCGCTACACTACCATCTTCTCCATCTATGAGACCCCTTAGAACTTGTAGCTTCTAAATCCACTAC
TATTCCTGTTCTTGTTTAGCATTCATTTGCTTTTGTGCATTTGTGTGTATGTGACGTGTGAGGGTATGTGCTGAGCCAC
TGTGTGGAGGGCAGAGTACAAACTGGATCCAGCTATTACTTGGTTCTGGGAATCGAACTCACCTTGCCAGGCTTTTGGC
TTTGGATTCTTCCTCCTCCTCCTCCTCCTCCTCCTCCTCCTCCTCCTCTTCCTCCTCCTCCTCTTCTTCCTCCATGTAT
TCATTCATTCATTCACTCATTGAGGACAGAATCTCATGGTCAGGCTAGCCTCCAACTTGCTATGTAGTTGAGGATGACC
TTGAAGTCTTTTATATATATGTTTTTTTAATTTTATTTTTTGTTTTTCGTAACAGGGCTTCTCTGTGTAGCCCTGGCTG
TCCTGGAACTCACTCTGTAGATCAGGCTGGCCTAGACTCAGAAATCTGCCTGCCTCTGCCTCCCAAGTGCTGGGGTTAA
AGGCGTGCACCACCACCACCCGGCTCTTTTTTATATTTTAATTAAAAAATTTTTTTTATTGTAGGCATATATGTGTTCT
GCTGTGAACATATGTCTGTGTGTCCCTTGCATACCTGGTTCTCGTAGAGGCCAGAGGAGGATGTTGAGTCCCCTGGAAC
TGGATTACAGACTGTTGTGAACTACCGTGTGGGTCCTGGGGTCCAGATCCTTTGGAAGAGTAACCAATGCTCTAGACTG
CTGAGCCATCTCTCCAGCCCCAGCCCTTGACCCCTTAAATGTTCCTGTCTGTCCCTCTCAAGTGCTGGGATTACAGGCT
TGAGCCACTGTTGCAGTAAATCTCCAACCCAAATATGCCCTGGCAATGAAAACAGGACACAGTTAATATGACTACATGC
TGTGCGCCTAGATTAAGCAGATAGACCGCTACACTACCATCTTCCACATCTATGAGACCCCTTAGAACTTGCTGTTTCT
CCACGCCATGTGCTTTGCTTTTCTTCTTCCTCCTCCTTCTCCTCTTCGTCCTCTCCCTCTTCCATTTTCTCCTTCTCTC
CCCCCACCTTTCATTCCACCTTCCCTTTATCTGCCCAATCATCAGCTCTCCTTCATTTTACAAATTAAGGTGGGAAGCA
GTTTTACAGGAAATCACCTGAGTGCTGGCTCATTCCTTGTTCACAGCCCCTCACAGGAGAATGGAATTAGCATCAAATA
TAATTAGCACCATGGCTATCCACAACTTGCTGCCACACCCCATACTGAACCACCATTATTTATCAATTTATCAATTTGT
ATATTGTACATGTTAACTCTTTCATAAACACATGATAATATTTTAGAAGACTTTTATGGATATAATGGTATATAATCTG
TTATGAAATGTATCTAACCATTCTAAGTCTTCATGCTTTCTTATAGTAAATATGTCTATCTTTGGGGCTGAGGAGGTAA
CTCAGAAGTTAAGAGCACTGTATGATCTTCCAAAGGACCCAGATCCAGTTTCCAGCACCCACATCTGGCTCACAACCAC
CTGTAACTTCAGTCCCAAGGGCTTCAACAACCTCTTTGGCATCCAAGTCAGTCCACATGTGGTGCATAGATGTATATGC
AGGCCAAACACCCATATACATAATAAAATAACATTAAAATTAAAAGAAGAAAGAAAAAGAAAATGGGCTAGAGACATGG
CTCACTAGTTAAAAGCACATATTCTTCTTGCAGAGGACCAGAGTTCAATTTCCAGCACCCACATCAGGCTGCTCACAAC
TGCCTGTTAACTCCATACAATCTGATATCCCTCTGACCTCCTCATGGAGAGAGACAGGCAAAGAGACAGAGAGAGAGTG
AGAGAGAGAGAGAATTTAAATGAATGTTTTTTTTTAATTAATCTATCATTTTTACCTTTCCCTGTTTAAAAAGAACTGT
TCTGCAGGATGGTGGTGGCACACGCCTTTAATCCCAGCACTTGGAAGGCAGAAACAGGCAGATCTCTGTGAGTTCAAGC
CCACTCTGCTCTACAGAGTAAATTCAAGGACAGCCAAAGCCACACAGAAAGACCTGTCTGGAAAAAAAAAAAAAAAAAA
GAAAGAAATAAGTTAATTTTTAAAAAAAAGCATTGGAAATGTAAATGAAGAAAATACCTAATTAAAAAAAAAAACCAAC
TGTCATACTTTGTATAAAGGACATTCTAGATTTTCTTACCCAAAGTTCTTGTTTTATTACTTTTTCAATTTTTGTGTAA
GTGTGTGAGATTTATGTGTAGGGTAAGTACACATGTGTGGAGCCCAGAGGATAACCGCTCCTTCTGAGCTGTTCACCTT
GTCTTCTGTGACAAGGTCTCTCTCACTGGGACCCTGAGGCTCACAGACTCAGCATAACCAGCGAGCCGCAGGAATCAAC
CTGTCCCCACATCTCCATGCTGGGATTACAAGTACCCATCGCCGTATCTGATGATTTGCATGGTGTTGGCTACTACTCC
AAGGTCCTTAGGCTTGCGTGTGGCACGCACCTTACTGACTGAGGTATCTTTCCTACCTGCCTTGCTTTGTTCTTTATGG
TTTATCTTTGATCAATCTAGATTTGTTTTTTTAAACAGATTTATTTAAGGATTTATTTTCTTTTTAATTTAGGGGTGTG
TGTGGGAGGGTGTGCACATGAGTTCCGGTTGTCAGGAGAGGCCTGAGGTGTTGGGTCCCCTGGAACTGGAGCTACAGGT
CCTTGTGAGCTGCCTGACGTGGGTGTGAGGACTGAACCCCAGTCTCTGCAAGAGCAACACTCACCCCTCACCACTGAAC
TGTTTCTCCAGCTTCCCTAAATTTATTTTGTAGGGTATAAGAGGCTGTGTCAAATTTCCTCTTACATTGCCTGTGCTAT
CAATAATATATCCCATAAGCCACTGGATTAATGATAAACCTATTTAGTCATAATGAGTACTCATGGGATCTGAAATCTA
TTTACAGATTATATAATTTATTTATAATTTTTTTGTCTCTGCCTGTGCTAGTATCATAATGCCTTGATTTCAGTGAACT
TGAATTGTTTTGATATCTGTTGAGGACAGACTCCTGTTATTCTTCACCTAGATATCTCTAGATATCTTAGACAGGTTCT
TAATAGTCATAGCTGTTCTGGAACTCTCTAAAGTCTACATAGACCAGGCTGGTCTTGAACTCACAGAGATCCACTTGCC
TCTGTCTCCATAGTACTAGGATTAAAGGTGTGTGCCACCACACCTAACCTGTTTTTTTAAGGTGTAATCTAAATTTGGG
GAGTATTTATTATTCCTCGGAATCATTTCCACTATTTTATTGTATTAAATAACCCTATAGACATATAATCTATCACGTG
TATATATTAGTTTAGAAACAAATGGTTGTTTATGATATTGTATCCCTAAAAAAAATATTGTTTCTTTCAAAAGGATTTC
ATGGTTCTCTACAAAAAGTGTATTTCTTTGTGCTAAATTTATTCTAAGCACTATGCAATTTTTGCTAGCCTTATAAAGA
AAATGTTTTCCCACTTTCATTTCTAGGTGCTTATTATCAAAATAAGAAGGAATATTTAGATGCACGTATCCTCTATCAA
GCTATTTCCAAAACTATTTTTTATTTTGGTTGGTTTAGTTTGGTTTTTGGTTTTTGGTTTTTTTTCCAGACAGAGTTTC
TCTGTGTAGCCCTGCCTGTCCTGGAACTCACTCTGTAGACCAAGCTGGCCTTGAACTCAGAGATCCACCTACATCTGTC
TCCCAAGGGCTAAGACTAAAGGCATGCACCACACACCTGGCTCCAAATTATGTTTTGTTGTTGTTGTTTTGTTTTGTTT
TTGTTTTTTTGTTTTTGTTTTTTGAGACAGGGTTTCTCTATGTAGCTCTGGCTATCCTGGAACTCACTTTATAGACCAG
GCTGGCCTTGAACTCAGAAATCTGTCTGCCGCTGCCTCCTGAGTGCTGGTATTAAAGGCATGTGCCACCACTCCTGGCT
CCAAAATTATGTTCTTAATCCTAGCAATCCTAAAGTCTTCCGGCTACTCTAGATGAGCTATAAGACAAGCTGTGGTATG
GCATTAGCTGTGCCCTCTAGCCTGGCAAGGTTCCACCGTCCTACACTGAACGTCCCTTTTGGTCTCTGTCACTGGTTTT
GTGTTGTGCTTTCAGGGTAACTTGATTGTGGATCCAGGTCATCTTTGCCACTGAACAGCATAATAATGACTGTTTTCTC
TTCCAATTCCTCTGATGAGCTGTATAATTGGAAAAATCGCTCTTTTAGTTCAGTATCCTTACTTTATATAAATAGCTGC
AGGTGTGCCCCCTACCACACCCCCATAGGTTCATTTGTCATCTGCCTCTTTTAAAAGCTTTATATCACTGGGGGTGGGG
TCCTTTGTCCCCCTCCAAATACCCTCTTACTCTCTAAGCTCACTGAGGTTCAGGTCTGAGCACAGGAGCCCCACTAAAG
AACGGTTGTTCTATCCGTGGTGAAAGGGGCAGTTTCTGCCCTGACTGTGACAGCTGCAGGCATGCAAGACTGTATGGCA
GACTCTGGCAGACTGCCCGTCTCCCTTGGAAGCATCTGGAAAATGTTCCCCTGGCCTGGCCTCTGAAGTCTCTCCCAAC
CTTAATGGCAAGAAGACTTAGTCTATTATCTGATACCTTTTCCGTTCTGGGATGGGACACAAAGTATAGATGACTTCAT
GGAATGTAATGGCAGCCTCTTCCTAGGGTCCCAGGTTTCTCCACAGTTCTAAAGCATGCTCTTCCTTACATTCCCTTCT
GATTGGGTGGTTTGTTTGTTTGTTTGTTTGTTTGTGAACTTGACAGAGCTAGAGTCGTCTGAGAAGGGTAAGCCAATCT
TGAGAAAATGCCCCTATCAGATTGGCCTGTGGGTGTTTTCTTGATGACCCAGTTCACTGTGGGCAGTGCTATCTCTGAG
CAGGTGGTCCTGAGCTATATAAACAACAGCAGCAGCAACCACCACCACCACCTAACTGAGCAAGCCATAGGAAGCAAGC
CAGTAAGTAGGGCTCTCCTGTAGCTTCTGCTTCAGTTCTGCCTCCAGTTTCCTGCTCTGAGTTCCTGACTTGACTTTCA
GTAAACCCTTTCCTCCCCAGGTTGCTCTTGGTCATGGTGTTCATCCCAGCAATAGAAATCCTAAGACAATCCCACTTCC
CAGTGCTTGCTTAAGCCTTACCAGTATTGACCGAACTCCTGGGTGAACAGTCAGCCCTAGCTCCCTCGCCTAACGCTGT
TTTTCTCAAGAGTGGCAACAATTCACTTGAAAGCACAGGAAAAGGAAATCACCTTATTGAACTTGGTGATGACAACCTG
CGCCCTAGATCTAACTATTGTCATCAATAGTCCAATAAAAAGAGACAAACTGATAGTGTGCTTTTGTTTGTTGTTTAGT
TTTCCTAAAGACACAGTCAACACAGTCTGCTTCAAAGTCCCAAACCTAGGTACAATTGGAACAATTGGCTCATAAAAAG
GCCTTCCTAGTGGTTGATATGTTTTATAAAACGGGGTATGATGTGGAGGGAGAGGGGTGCCTTAGCGGGCCCATGCTGA
GGCGCTCCCCACCCCCAATACACACACCAGGTGTCATATCAGCCATACAACGGGTGTAGCATAAAAGGAAGTTTATTTG
GGACGTGGGAAGGGGTTCTGAGGAAGGGAGCAGAGGCAGAGGGGAAAAAAAAAAAAAAGAGGACAGAGGGGGACAGTAA
GAGGGAGGAGAAAGAAGAAGAGGCCTCTAGGGAACAGGTGGGGAGAGAGAAAGAAGTTGGAAGGGATGAAGGGAGGAGT
AGATGGAGGGAGGGAGGGAGGGAGGGAGAAAGGGAAGGAGAGAGAAAAACAGACTGACAGAGACACAAAAAAGATGGCG
AGTTCTTTTTATCTACGAGCCAGGCTCATACCTGGCTGTTGCTAGGTAACTGCTAGGCGGAGCCTAGAGGAAATGCTAA
CACCTAGCTCTATGAAAGTCCCAGCCTCAAGGGATGGTCAGAGGGTGCTGCCTTCTGCTACCTCCCATGTCAGTTTGAG
ATTTAAGATAAAACACAGAGCAGAGGGGGTAAGCTTAGTAAAACGGTTGGTACTGAAGCCAATCAATCTAGCATACATC
CGTCCCCTCCCTCAAATCGCAGAGCCATGGCGGAGCGAAACCCACCTCAATCTTATTCAGGGTTCTCTGTCTTTGCCCT
GCCCGAGACACCCAGTCTCTGGTAGCTTAAGATGCTGGCTGAACATTTCTCCCAAATCCAAATGGTTGAGAGAGTGGGG
GCGGGGGGGGAGCGGTACGGTCCTCAAGCGCCATAAAAACAAAACAATACAGCCTTCTTCTGCAGGCGTCTGCTCTTGT
CAGTGCCCTCCTCTTGGCCCCCGGAGTGTGCTGAGCGTGTGCACGAATGTGCCCAGGAGCCCAGAGCTCCCGTAGCGGG
GCGGGGGCAAGCAGGGCGCTGCGGGGGACCAATGCCGCACAGTAATCGGCTCTCCTCTGCTGGCGCGTGGCCGAGTACC
CCCACCCCGAGGGGGTGCGTTCGTTCGCGGTGGAGAATTATCTCATTGTTCCCAGACTCAGCGGCCCCCCACAGCTAGA
GATCTTTTCACCCTGGCAGCCGGGCGTCCGGCGCTCAGCCGCTCCCCGCGCACCTGCCCTGCGCCCCCGCCCTCCCGCC
CGCCATCTGTCGTCTGAACGCCATTGTCAGCAGCCACGCCTGGCGTCCTGGACGACACCCAAGGGGACCCGGGGCTTAA
GTCCCAGAAAGAAGCAAACAAGGAGGGTTTATTCCAGTCCTGTTTGGAAGTGACCTTGAGTAAAATAGACAAGTCTGCG
CTGGGAGTGGGGGTTGGAGGAGGGAGGGTCCTGTTCTCCCGTCTGCTGACTTTTCCAAGGGGTTTCGTTGGGGGTCAGA
GGGTTGAGGGTACACAACTGGGCTTTTCGGAGAAGCTTTTCTCTCTAGGAGGAGCTACTCAGCAGGCAGATCTCGCAGC
GAGCCTCCCATTGGAGCCCCGGGTGGGAACCCATTTCAAATGCAGATGTTCTCTTTGTCTGCCTGCGAGTTTGCCCCTC
TGGTTTACCCCAATTGTCGCAGCCTCTGGGGATGCCGGGTTGAAAGAAGAACCTGTCTTTTCTGCCATCACAGGTGCAG
CGCTTTCTGGACCCACTAGCCGCCTGCCGCTCGCCTCGCTGTTCTTCTCCCAAGTTTGTTTACCGTCTTCCATCCTGCT
TTACCGTTAGAGGAAATTTCAAACACTTCCCTGAGTTTGAAAACAATCGCAAGCAATCCAAAGTTGCAGACACTGAAGT
CATGGTGACCTCACCTTTCTAAATCTCTGTGCTCACAAACCTAGGCGCCTCCTCATCCCCTGTAGGGACCTTATAGAAC
GCGCGCGCGCGCGCGCGCACACACACACACACACACACACACACACACGCACACATCTCACTCCCATGCAGTCCATCAA
CGTTTTCCCTGGATGGGAAAACAAAGCCCCAAAGACAGGCGATAGTGTGGACAGATGTGAACTTTCACCTCATTGCACA
AAAGACCCATTGGGCTCACTGCTCAATTTGGATCTCTATTAGTAGCCAGCACACATCGTGCAGAGGGAAGGTCGGGGAT
CAAGGGGGCCTTTGGGGATGGTGTCTCCAGGCAGACAGCTCAATGGGAGTTACTACTCCCTTCCTGGAGGTCTCTTTAG
TCTGCACCAAGAAAGGAGAGTGCACCCCTGAGTGTCCACTCCCCCAGGAGGCCCACTTACCTGGCTCCAGCTGTCTCCA
CTGGAGCAAACTGGGACCTGCTCTGTTAGGCTGGGACCCTGGAAAGACCCTGCCCAAAGCTTGGGGGTAAAATATTTAC
CAGACCAGAATGCATATAAAAGTTTTAATAAAGAGTATGCACTGTGGGAAGGAGAAACACAGCTGTCGGAGTTCTCAGA
GGGAGTCGTGGCAGAAGAGTGAAGAGCAAATGGGGCCTTTTGATAACAGACAAGAGAAGTCTCACCCTGTTTTATGCTG
AGAGTCACTTTATTTTATGACACCCTTTGTCCTACCTGAAGCAAGTTTTGGGGGTGAGGTGTCTCAGGCCATTCTGAGA
GAGGCTGTAGTTATTTAAAGCATTATTGCACTAAGGTTCCTAATGGGTTAAAGGTAGGAAACTTTTCATTTTCTTCTTT
CTTTCTCTTTCTTTCTTCCTTCCTTCCTTTCTTTTTTTTTTTTTCTGGCCTTCTTTGACCCTAAATCTTCTAGAATTTC
CATCAATAATTCTCCCTACAAACGGGGTACGGGGCGGGGCGGGGGAGAGCATGAAATCAGACTGGAGGGAAATACTGGG
AATGCCGATGCCTGGTGTTGTATGGACTTTCTTCTGGAACAGAACCGAATCCCTTTCACAGAGGGGTATCCTCTCTACA
AGCTCAGTGCCCACACCACCTCCCCGGGTTTCAAGCACATATGTTCATGCGCCTCAAATGTGTGCAGATAAAGAGGTAC
ACCTGGTGCGCACACCACGTGGTAAAAAAAGAAAAACAAAACAAAACAAACAAACAAAAAAACCTAACAAAAACCCCCA
AAGTCTCCTTTCACAGGTTTCCAGATGGCAAGATCTGTTCTGCTTCCCTGCTCGGCCTCCCAAGAAAGGCAAGGCTTCC
AACTCAGACTCAGGGTCTCTAAATTTGGCCTTCCTCCTCAGGAAAGGAGCAGTGACAGACAGTACTGCAGAAAGACTTA
GGAGCCGATGGGAAAGTGCCAGGCCTGGTAAGCGGGTGACCCGCTCCTGAGTTACCTGCTGAAGGCAAGACCCAGTTCC
CCACCTTGTCTAGTGAGATCTGAGCGGAGAGGGTTAGTGGCCTACCATGAACAACAACAAAAAAGAGGCCTTTTTCTTT
CCTTTCCATTTCCTTTCTTCCTGCCATTTTTATTTTGTTTTGTTTTTGAGACAGACTCCCTGGTTAGCCCCAGCTGGCC
TTGAGCCCCCCTCCCCTCCCCCCTCCCCCTCTCCCTCCCCCCCCCCTTCCCAAATGCAGGGATTACAGGGCTTGAGCAC
TGCACCTGGTTAAGGCCTCTTTCTGGTTCAGCATTGTAGGCCAGCTTCCGCTGCTGAGACTGGGGATTTGTAAAACAGC
TGGGGCACTTCTGGTCACTGTATCAGGTCCCTGGGCCTGGAAGAAGGCCCAACTCCCTTGAGGATGGTCTTCTAGCAGA
AGCATTAGATTGCTAAACTCATACAGCACCGAAGCTGCTGGCAGTAACTGCTCAGATAAGTCACGTGGCCAGAGTTACC
GTTAACAGCTGAAACGTTCCCCTCAAAGAGTTGTGTGTTAAGGGTCTGACTGACAGCTGATGTACTGACTGGATCATGA
GGACTTGGATGTAAGCAAAGGGTTAATTCTCTGAGAAGTTCTGAACTTGACCTGGCCCTGTTAAGGGGCAGGGCCTAGT
TTAAGGAAGTAGATCACAAGGGGTTTATTATGGACAGGTGTGGGAGCTGGGGGAGGGAATTGCCCATAGCCCCGTCTCT
TTGTCTGTTTCTAGGTTTGGCTGTTACAATGTTCACTTCACCTCTTGTCCAAAGCAACAGAGGTGAATAAAACCTGTGA
AACCAGGAGCCAAAGGCATCTCACCACAGTTTAGATTGTTCATATTGCGTTTGTCACATGATGAAAGGTGACCTTCACA
TAGCCCAACCCAAAGTCAATGTTCACTTCTGACAGGAAGCAACACCGTAAGCTGAACCCTCCCAGGAAGCAAAGAACCA
GTATTTCTGTGTCTTCTCTGCATCGTTTTCTGGGATGACAACACCCATCCCTGGATCCTAAGTACTAGGATGTAGTTTG
GTCTCAAACTCACTGTGGAGCCTTGGATGCCCTTAAACCTCTGATCTTCCTGCTTCCATCCTGGAATGCTGACCTTACA
AGGGAGATTCTCCACACCTGGTTTATTCAGTGCTAGGGAACTGAACTTCCTACGTGCTAGGCGAGCTCTCTACCAACTG
AGCCCTAGCCCTAGATTCTTTGAATCTTGATGATCCCCCAAAGGCTCTAGTGTTAAAGGCTTCGTCCCCAGAGTGGTGA
TATTAGGAGGTGGTGGAACCCTTCAGAGAAGGAGCCGAACTTGACCTGGCTGTTAAGGGATAAGGGGATGTGGACCTGT
TGTCCTTCTGGTGTGTCTGAAGACAGCTAAAGTGTACTCATATAAATTAAATAAATAAATAAACAAATAAAATAGACAA
GAGCTAATAAATATTGAGCCGTTGTTGTGTCCCAGATGCTGTTGTAAGCATTCTGCCAAAACAAATTCCTTTAATCTTA
GTATTACTTGTACCCATTTTACAGACAGGACAACCTGAGGTACAGAGATGCTAAGTGACAGGTTTGAGCTCCATAAAGG
AATGACAGTGCCAGCGTCCTACCTGGCATCCTAAATGCAAAGGCATGCAGTATGGCAAGTGAGACTTGCAAAGATGATG
TGGCCACAGGGATACAGAAGTGGGCCTGTCAAAAGGTGGGTTCTTCATCCACGATTCTGGCTGGCTGGCAGCATGCACC
CATCATTCAGGGCCTAGTGAGAGGTCCTTGGGTCATTGGAGATATGTCCTTGAAGGGGATTGTGGGACACTAGTGTCTC
CTCGAAGTGGGAATTTCTGGTTGTCATGTGATGCAGACTCACGTGATGTTTTTGCTGAGGTAAGACCCAGAGAGGACAC
ATGATGTTTGGAGAGAGTATAAGTAGGACTCGACGGACAGTGACCAGATGCTTGCATAGCTAGATGTGCAACTCTTTGT
TGGTCTTCTGTCTAAGCTGATCCCTGCTTTGTTGAGAGAAGCACGGCAGAGAACTCCTGGCGTTCCGGCTGATCTTAGT
TGTTCCCACTGACTCCTGCCTATCCGACAGAGACCTGCCGGTTTCTGCTGGATCGTGACACTTTTGAACTGGATTGTTG
GTATCCTGACACTACTGAACCAAACTGCTGTTATCCTGACTAACGGAGATTGGAATCACCTCCAAAGAACTACTTCTAA
ACAGGTCCACATCCCCTTGTCCAATTCACCATCTTTTCTCCTCTGCCTTTGGACAGTGGGCTAGAAGGGGGCTCAAAGC
ATTTGAGAACCCTTATTGAAGTAGGCTTGGAAAAAAATCTAAGCCTACTACCCCTCCTCATGCTCCCATTTTGCTTTCT
GGTGATGAACTGAGCAGTTTCGCTCCACCACACACTCCTGTCTCGTCAAAGCCTAAAAGCAGTGGGTCTGCCTACCTAT
GAACTGGACCCTCCAAAACCATAGGCCAAAGTAAACCTTTTCTCTTTATACGTCCACTAGCTCAGATATTTTATTACAG
TAGTGGTAGGATGACTAACACATGTATTCACTGACGATTTCCTAACTTAACAGTCCAGTCTGAGCTCCAAGTACCCCCA
ACATCCTACAATTCCCACTCCACCCACACTCCCACACCCCAATGCCTCCACTTGAACATCTCAAGACACCCCTCCTTGG
AGATTCTCCCTGGACTACGCCCCAAGGACCTCTTCCCTCGATAACTGCTGTATTTGTATAGCCCTAATGCATTCCTTCT
GTCTCATCTCAAGATATGGAGAGAATACTCATGAGTTAAAGGTGAGATTCTGTAACCCCTGTGTGAGCCCCATTTCAGA
GGGAAGTGAAGCTCCTTATAGTGCTCCCCGAGAAACTACACCTCTCCCTCATCCCCTGCTGCTTGGCCTCAGATTCAGT
TCCAGCCACAACACCCTCCCCTGTCTTAGGGTTTCTATTCCTGTACAAACATCATGACCAAGAAGCAAACTGGGGAGGA
AAGGGTTTATTCGGCTTATACTTCCACACTGCTGTTCATCACCAAAGGAAGTCAGGACTGGAACTCAAGCAGGTCAGAA
AGCAGGAGCTGATGCAGAGGCCATGGAGGGATGTTACTTACTGGCTTGCTTCCCCTGGCTTGCTCAGCCTGCTCTCTTA
TAGAACCAAGACTACCAGCCCAGAGATGGTCCCACCCACAAGGGGCCTTTCCCCCTTGATCACTAATTGAGAAAATGCC
TTACAGTTGGATCTCATGGAGGCATTTCCTCAACTGAAGCTCCTTTCTCTGTGATAACTCCAGCTGTGTCAAGTTGACA
CAAAACCAGCCAGTACACTCCCCACTGTGCTGTGTGAATGGAAGTTCCCAGAAATAAGCTGCACCTGTGTTAAAGATGC
TGCAAGGCTGATTGTTGGATTCGAGGCAATATGCAGAACTGGAACCAAAACCACTTTGAGCTATCCTCAGCCCTCCTTA
ATAAACCACTGCATGTGTTACTTTGCTCTTGCTGTGTTTAAAAAAACACAACACGGGGGGCTGGAAAGATGGCTCAGCG
GGTAAGAGCACTGACTGCTCTTCCCAAGGTCCTGAGTTCAAATCCCAGCAACCACATGGTGGCTCACAACCATCTGTAA
CAAGACCTGACGCCTTCTTCTGGAGTGTCTGAAGACAGCTACAGTATACTTACATATAGTAAATAAGTAAATCTTTTAA
AAAAACACAACACAACAAAACAAAATGAAAACCTATAACCAGATCATCTTGCAGAGGGAAGATTATTTGGTTTTAAGGC
TCCAGAGGGCTCAGAGTCTATCACGACAAGGGGAGCATGGCAGCAAGCGGCAAGCATGACAGCAGAGCCAGGAAACTGA
GAAATCACATCTTCAAATGCAAGCAGGAAGCAGAGAAAGTGAGCTGGAAGTGGGGCAGGATTGTGGACTCTCAAAGCTC
ATTCCCACCGTGATCTACTTCTGTCAGCATGGCCCCTCCTCCCAAGGCTCCACAACCTCCGGAAACAGTGCCTCCAACT
AGGGACCAAGTGTTCATCTATGAAAGCCCAAGGGCGACATTTCTCACTCAAGCCATCCTGACTATTTAGTGCTCACCGT
TGGTATATAACCAATGTCACTTCAACGATTAGGGAAAGCCTTTGAATATGTCAACTTAGCAGGCCACAAGCTTGTACTA
ACCCTGAGTGAAGTTTGTAATCTATTCTGAGAGCATCTGAGACATAGAGCCAAGAATCTCTGCTTTGTATTATGGCCCA
CTCCATCAATGTGTATGGTGTTCCCTATCATGTGTTTGGTAAATACTTTCATTCATGGCTTCTTTCTGTTCTCTGACCA
TAACTCCGTGTCACTTGTGGTAGTTTGAATATGCTTGGCCCAGGGAGTGGCACTATTAGGATGTCTGGCTTTGTTGGAG
GAAGTGTGTCACTGTGGGGATGGGCTTTGAGACCCTCCTCCTAGCTCCCCAATGAGACAGTCTTCTCCTGGTTCCCTTC
AGAATAAGATGTAGAACTCTCAGCTCCTCCTGCACCAAGTCTGCCTGGGAACTGTCATGCTTCCCACCTTGATGACAAT
GGACTGAACTTCTGAACCTGTAAGCCAGCCCCAATTAAATGTCCTTTATAAGAGTTGCTTTGGTCACGGTGTCTCTTCA
CAGCAATGGAAACCCTAACTAAGATATCACTACTCACATGTTGACACATGTTTTTCAGGGGACCTGTATCTGTGTTGCC
AGGCCAAGGACACTCACATTTGGCTCAGAATAAACTCCCTCTTACTCCCTTTGTGGTGAGAACTGCACTTTATATTGAC
ATCTGCAGATTCTGGGGATAATGGGCCTTTGCATGAACTGTGCCTGCTTCCTGGAGAGCTACCTCTTCTCCACACACGT
GCAATACTAGAGCCTTACCTCACTCTCTCAGAAGAGCTTTCTCATCCAAAGCTTAATCTAACCCAAACCTCCATCCCCC
TTTTGTCTCACCACCAGACATATGAAATGGGTTTCAGTTTGTTTTGCTTATTTTCTGCCTTTAGTGAGGATAATGTGTG
CCTGCCCAGTGCACAACAGATACCCTTACAGGGTGGTCTCAGCAAGGCAATTACATGGAAAATGGGGCTGATAGATTTT
CCTGTGATTTGTACACTCCTCTATATCTGTCCCTGGTGCCTTAGTGCAGTGCCTGGTGACCAATATATAGAAATTGAGT
GAACGAATTAATGAATGAACAATTGGAGTGACCATTTAAAATTTAAATCTGATCCTATCACTTTTCTTGGAAAAAATCA
GGGTTGGTGGTCTTGAGAAATGACTTAGCAGTTAAAAGCACAAACTCCTGCTCATCTAGGGGGCCCAAGGTTGGTTCTC
AGTGCCTACACAAGGCTCGTCAAAACTAATAAAACTCCAGCTCCAAGGAACTTGCTTCTCTCTCTCTCTCTCTCTCTCC
CTCCCTCCCTCCCCTCCCCTCCCCTCCCCCTCTCTTCCCCTCCACTTCCCCTCCCCTTCTTCTCCCTCCCATTCCCTTT
CCCTTCTCTCTCTCTCTCTCTCTCTCTCTTCCTCTCTCTCTCACACACACACACACATTTAAATAAACCTAACTTTAAA
TAGAGAGAGGCAGGCAGATCTCTAGGAGTTTGAAACCAGCCTCGGTCTATCCCTGGCCAGCCAAGGGCTATGTAATGAA
ATTCTGTCTCAGAAATAAAATAAAAATTAAATCAATTTCCTTCCTTCCTTCCTTCCTTCCTTCCTCCTCCTCCTCCTTC
TCCTCCTCCTCCTTCTCCTTCTTTTTAATCAGCGAGTGGTTGGCTCCACGTCTTACTTAAACCAAAATCCAGAATCCAC
CTTTGGAGCCTGCCTCATCTGGTCCCTTCAGGAAGAATCTCATACTTCCTCCCCAGCTCATTGAATTCTGCCAGGCCTG
ACCCCACCCGCCTCCTAGCCTCTGGCCACCTGCTCACTCCAGCCCCCTAGTCTTAGCTGGTGCTTCTTCTTTGTTAGCT
TTCCTAGGAGGCTCCAGTTCCTCCTGTAAAACTCAGTCCTAGGAGGGCCACTTCATCAAGACCTCTCCCAGGCTACCCT
ACCATGAGGTGCTGCAGCCTTTCAGACCATTCACTGCCTGCATGTGCTGGTCATGGACTTTAACTGCCTCTCTTCCCAT
TTGTCTTGTTCTCTATTGCAATGGTACTAGAATGCACGGTGCACTCTTGAGCATAAGCTGGTAGGAGAGGAGCAGGCAG
AGAGACTGGGGAAGTCAATGCCTAAGTAGGGGGAACTAATTTTACTGTGCACTATACAGGTTTTTCAGACATTGGAGTT
ATTTCCTTGGACCTTGTCAATGAAGACACTGATTGGAAAAAAAAACAATGTATCTTTTCAGCATCTAACTGGGCCAGGC
AGTCTTGAGCACATCTATACCTCATCCCATTTGACCTTCACAGCAATCTTAGAGTTCTTTCTCTTTCCAGTGCCTGTCA
TGACACCCATTCTCCACTGCTTCTTCGTTAATAGGATCTGGTTTTCTATGGAAGGAGAATGTGTCCAGCCCTGGTTGAT
TAATCAGACTCCCTTGCAGCTAGGTTTAGCTATGTCATTCTATTGTGGATGATGTGGACTAAAAGCAGTCTGTTGGTGA
TGCTGGGGTATTCTAAGAAAGCTTCTGCTCCATTTACATCTGTCTTCCTGCCTTAAAGTGGTGTGTTATCTGGAGTAGT
AACAGCCAACATGTAACTGTGAAAAAGCAAGCTGAAAAGTGAAAGCCATCTGACTAAACCTAGAGGACTGGAAAGAGGA
AACACTTTGCAATTATAGATACATTGTTAATTAGATGAACTAAACCCACCAGCAACTGTCATACTTTTTGCTAAATAAT
TGGATTAGGCACCTATTCGTTTAAGAAACCACTACCAAATTTGCTGTCACTTAAAGCAGAACACATCTGAAGTTAGTAA
GGTATGGTGTATATGATGCATGTATGTGCATGCATGTGTGTGTGTGAGTAGTGTGTGAGAGTGTGTGTAGTGTGTGAGA
GTGTTGTGTGTATAGATGTGTTTGTGCTCATGACACCACACTTAACAAATGTGTGTGTGTGTGTGTGTGTGTGTGTGTG
TGTGTGTGTGTGATCAAACTTAGGTTCTCACATTTGGCCAAGAGTTCAAATGGGGTAGTCAGGACCTTACCAGTTCTCC
AAATCTCTGGAACTCAAGCTTTCTCTACCCCACAGGCTGTTTCCCAGATGGCCAAGTCCTCACCAGAAGGGACTTTGAA
TATGAAAAGTATGCTAGAGTAGCTGGAATTCTTCATATAAATTCCAGGAAATCCAGGGATTCTGGACACCACATGAAAA
TAGAAAAGAGGGAACATCACACACACACCCCCTTTCTGAGCAAAGTGAATCAGACTTCACTTTCCTTGATGCTTGGTAA
GGTTGGGGGTGAGTATTGGAACAGGAGACACCAGTTCTGCCCACCATTCCTGCCAGAGCCTCGGGTGGAGGTGGACATG
GTGCCCCCCAAGGCTGGTAGAGTATGGCACGGAGGAGATAAAGAACCTATTGGCAGGCAGGGATGGGACAGGCAATCTG
ACACAGTGCTCTCAGTCAGAGATGTGGGGAGTTTGCCTGCTGGAGCCCATACTATCACCCATCGTATTTCCTGGCTCCC
AGAAAGAGAAAGCTTGGCTTTGCTAGTCACCGCCCCCTGCCACGATCCATACACTCACAGCTATGCTGGTTGTGGACCA
GGGACCAAGCCAATATCACTGTTCTCTTCATCCAGACAAGTTGTAAGAGCAACAGAGACTGCCAGTTTGAAACCAGGCC
TCAGCCATGTTATGGCTTCCTTCCACACAATGAATACCGGGTGTCTACTGCATGCCAGGTAATTTTGTAATCTCCTGGA
ACCTTGGCCTTACATTCTAGTGAGGACATAAACTGGACATACCAAAATAAGTGGAGTATGGTGTGCATCGGGTCCAACA
GCTGTAAGTGGAAAGAAATGGAGCAAGGACTGGGGAGCTGGAACAAAGAAGGGGCATGAGCAGAGGTGGGGGGGTCAAG
GTCAGAGTGACCCTTTGTGTGCTTTGTCTCCTATGATGGGTGTCCCCCTGATCTAAAGCAGATCAAGAGTCCTGCTGAG
TTCTGTCATTCCCCAAGTGGCCATGTCCAAGGCCCTTTCTGGTCCAGAGGAGGTCTGATGCAGCTGCCCCCCCCAATCC
AAACCCTCAAGGCTGATGCTAGCAGCTGCTCACCTTTGCTGGAGGGGGTGATCTAATTCACCCCTCAGAAGGGAGTACT
TTGCTCAAGGTCCCTCTGAGAATTGTGACCCCTAAACTTCGGGAGGTCCTGCGCTAGTTTGCAGTGTTTTCTGAGCAGT
CAACAAGCATCTCTATTTGTCTAGAATGGTTTCCAAAGCACCAGCTGTAGCTTAATCCTACGTTCCCCTGATTAATAGA
TTCCCTTCGTGCCAATTCCCATCTACAATGCTCAATACCATTCTAACATGGCTCATACTCACAGGTTAAGCTCTGGCCC
ATAGAGACCCAGGAACTCCCTCTTCTTAGTCTGAGATGGCATACCCCATGTGCCCCGCAATGCATGGGAGCCAAAGTGT
TTATGTTTCACTAGAATCTCCAAAGGCTGCTGGAGTCCCTGCAGCCATCCTGGGCAAGTATCTCCTGTTTTTGTTTCTT
CTGGCCAGCTCATGCCTGAGCACACAAAAGGTGTGCCACAGACCCAGCTCCTTCCTCCAGGAGGAACAAACCCCTGCTT
TGTCCACACAGCCACCTCCATGTTTGCGAAATGGAAAGAGGTTGGAAAATTCCAGCATTGGAGTGTTCAGCCTGATTTC
AGCCCTGTGAGTGGGAAGAAGTGTGCTTTTCAAACATTAAAAATTTCTCAACTTTTCCTCTGAAAGCAGTTCACAACTC
CCTGAGCACGTGCTCACAGGAGAATGAGGAAGGCCGAACCAGAGCGGAAGCTATGCAGCCTGGAAAATGAAGCTATGCC
AGAGGAAACACGGGAAGCTCAGCGTCTGTGGTCCTGGCTTGCCATTTTGCAAAGGATGCTTCATGCCTTTCCTGTATCG
ATTCTTCTTTAATTTCTCCCCAAATTCACTCACTCACTCATTCACTCACTCACTCACTCACTCACTCACTCACTCACTC
ACTCACACACATATTCATTCACTCACCCAATCACTTACTTAGTAACACCTTTACACACTCACTCATTCATACACTCACT
CATTCACACCTTCACACACACACTCATTCACTTATTCACTCACTCTCACACACATTCACTACATATACACACACTCATA
CACATACTTACATACACACACACTCACTTGCATACACACATTCAATCACTCTCACATATATACTCACTCACACACACTC
AGCCACACAATTCACTCATTTATACACTCACTTATTCAATCACTTTCACACATACTCACACACTCAGCCACTCTCACAC
ATATACTCACACACATACTCACTCATACACTCACACATACACACACTCACACTCACTCATTCACTTACTCACTTTCACA
CACTCATTCACATACACACACCCATACACACACTCATACACACACTTGCATACATACTCACAAACACACTCATATACTC
ACTTGCACACTCACACACTCAGTCACACACACTCACTCATTTGTATACTCACTTATCCGATCATTCTCACACACACACT
CACACGCATACACACAGCCACTCATTCACTTATTCACTCACTCTTACACATACACTCACTCAAATACACACTCACACAC
ATACTCACTTACACACTCACACTCACTAAGTAACACAGGTTCACTCATTTATACACTCCCATATCAAATCACTCTCATG
CATACACTCACACACTCAGTCACTGACACATATCATCACACACACTCACATGCACACACTCACACTCACTCATGCATTC
ACTCAGTTCCACATTTGCATACACTCCCTCATTGACACAATTCCCACTCATTCACACACACCCTCATTGACACACTAAT
CCACTCATTCACTCACACACTCACTTGGACATTCATTCATTCATTCTCTCATTTGTCCACTCACTCGTTCACTAACAGT
GATAGAAAGTCAAACATGGCTGGCAGCTTCTAACAGGGCTCCCTGTGGACCTCACTTCTGGTTTCTATCTTGCACAATC
CTCTCCCCTTGACAACCCACCACACCTAGAATCACATTCCTAGTAAAAAGAAGATGGTGAAAGTGACAGATTCGTGGTC
ATAAGGCAGTGCTAACGATACTAATTTCAACTTAGTTAAAACTACAAGGTTGGCTAGCATATGAAGCCAACGATGAGGT
GTTATAGCCTGACAGGCCATAGGACCTGTCACCTCAAAGGAGCTTAATAGGTTCAGACCCATGTCATGTGTCTAATATC
TGAAAGAACTACTTCTCACACCTAGAAGAACATTCCTGTATGTTCTAGAAACAACTTAATGAGAATTCTGGCTTCCTTC
AGGCAGCCACCAATCACAAAACAACTTGGATCTTCCTATGGGGGTGGGGAGGGATCACAAAAGTCCAAGTTTTCCTGCA
CTCAGCAAAAAATTATGGAACCCTCTGGTAAATGCCAAATGTTGTTCTACCTATTAGGGATACAGCCAGTGATAAACAG
ACAGAAGTCTCTGGTCGTGTCCAGGTTATGTCAAAATTGAGAAGAAAGCAATGGGTGGACAGCTGAGACAGACAACCCA
TAGAAAGGCAATGAGTTGACATAGGAGAAAGTGGGCAGCACAGAACAGGGACCCCTGGTGTCTTAGGGTTTTACTGCTG
TGAACAGACACCATGACTAAGGCAACTCTTACAAGGACAATATTTATTTGGTGCTGGCTTACAGGTTCAGAGGTTCAGT
CCATTATCATCAAGGCAGGAACATGGCAGCATCTAGGCAGGCATGGTACAAGAGGAGCTGAGAGTTCTACATCTTCATC
TGAAGGCTGCTAGCAAGAACACTGGCTTCCAGGCAGCTAGGACTAGGGTATCAAAGCCCACCCCCACAGTGACACACCT
ACTCCAACAAGGCCTCACCTCCTGATAGTGCCACTCCCTGGGCCAAGCATATTCAAACCACCACAGTCTCCCTGGGAAC
ACCATCTGAATCAGAACCCATCAACCACGACAGTGTATTCCCTATAAACAGGATAGAGGCAAGGACTAACTCCAGCAAG
AAAACCTGTGCTGACACAGTGCATTTCCCATTCTTAACTCACCTGTGAGGCCTGTTGGAACTGATCACAGGAATCCAAC
GGTAAGGGGTCTCTCCCCCGTACTTTCACCCTCTGCCTGGACAATGTCACTCAGACACATAGTCCCAGGTGGCAGGGTT
GAAACAGGAGAGCCATTCAATGGCATCCCCCTGACACTACTGAGAAGAGATGGTAGCTGTTTGGTGTTGGGGCATGCCT
TTAATCCCAGCACTAGGCCGAGGTAGGTGGAGCTGAGTTCCAGGCCAGCCTGGTCTACAGAGTTCCAGGATAGCCAAGG
CTACACAGAAAAACCCTGCCTCAAAAAAGAAGAAGAAGAAAAAAAGTGTAACATGTTTGTATTTGTGGCACCACATGTG
AGTGTGCAGGGTGTGTGCCCATGCACAGCACCTGGGAGTCTTCCTCTATGGCTTTCCATATTCTTCACTTGAGACAGGG
TCTCTCACTGAACTAGCTGCTCTCCGTTTTGGTTAGGCTGGCCAGCAAGCTCTCAGTATCCTCCTGCCTCTGCTCCACA
TCACTGCCAGGGTTACAGAAAAGCTCAGACAAGCTGTACTTTTTTATGTGAATGCTGGGAATTCACCCTTACATCCAGG
CCCTCTGCTTGCAGAGAAAGAACTCTTACTTGCTAAAACCATCTTCGCTGACTCAGATTTAAAATGACTTGTTCTGGGG
CCCGGGAGATGGCTTAGTTATAGAGCTTGCTGAGCAAAGATGAAGTTTAGAAGAAGTTCGACACTACTTTCATCAAAAC
AGCTAAGCGCATAAGGCCTATAATCCCAGTCTGGGGAGGTAGCGGAGAATTGGATGAGCCCTGGAGTCTTCAGTCTAGC
CAAATCGGTGAGCTCCAGGTTGAGTTGACCCTTATACCCCACACAGATGCGCACATGGATGCAAATGCACGTGTATGCA
CACATACACACAGATACACACACATGCACCCATCCACACACATACAAAGGTATTTTTAGAGATTTAAAAATCACAGAAA
AAGGCTGGCGTGGTGGCACACACTTTTTTTTTTTCTTTTTTGGTTTTTCGAGACAGGGTTTCTCTGTATAGCCCAGAAC
TCGGGAGGCAGAGGCAGGTAGATTTCTGAGTTCGAGGCCAGGCTGGTCTACAAAGTGAGTTCCAGGACAGCCAGGGCTA
TACAGAGAAACCCTGTCTCAAAAAACAAAAACAAAAACAAAAACAAAAAAAAAAACAAAAAGCAAAAAACAAAAACAAA
AAACAACAACAACAAAATCACAGAAGATATTTAAATTCAAAATGATATGTTTATATATATGTATGGTTTTATTTGTTGC
ATTGTGTTGGGACAGAGTCCAGGATGGCCATCACACCCAGCTTCAAAATGTCAAAGGCATAGATAAAGTATGGTGGCAA
CTCCAAGTTGCTATTTAATGTGCCTTATATTACAAGAGAAAATACCAATCTCTGACCTATGGGGTGGTAAACAGACATA
CAAGACAGTCAAAGCATGCTGTCCAATATCCCACTATACCCTGGTCTAATATTCATAGCCAAAAAATAAACATGCAAAA
CAGTATCTCATTTAAAAAATTTAAGAAGGATTTATATTTAACAAGTGTGGCAAGCTGAGATTCCTTTTCTCTTAAAATA
TTTCTAGAACTATTAACTTACTAGAGAACTACTAACTTAGTAAGGAACTGGTCCTTTACCAAATCATTGCCATAAAATA
TCCCCCTGTGGCTGTGGATTTGGTTCAGTTCGTAGATTACATTCTCAAGGCCCTGGGTTCAATCCCCAGCACCGCCTAA
ACCAGGCACGGTGGCACACACCTCTAACCCCAACACGGAGGGAGTAAAGGCAGAAGAATAAAGAGTTCAGAGCCATCCT
CGGTACCTAATGGAGTCCGAGTCCAGTCTGGGCTACGTGAGTCTCAGAACATTTTTTTTTTCTCTGTATCATCATACAA
GAGGGAGATGGGACCACTGACAAAGCCGGGCAAGATTATCATCAGCCTCGGCTTCTTCTGCTCCTGCATCATGAGGGAC
TGGATGCTTATTATTATTATTAATTATTACATTTATCTGTGGGGGTGGCGTTTATGCATGTGACATGGTACATCTGTGG
AAGTCACAGGACAACTTTTGGGAGTCAATTCCTTCCTTCTACCATGTGGGTCCTGGGGCTGGAGCCCAAGTCATCAAGT
TTGATAGCCAGTGCCATTCCCTGCTGGGCCATCCCACTAGAGCAGAGGCATCTTGTCCTTGCAGTTGCTCCGGGAAGTT
CGTCTTTGGATTGTGTGTTAGCCACTCTGACCTGTTCCCCTCCTGCTTCGAGTCACTGTGTTGTTCTAATAAAGCAAGC
AGCAGGGGAGAAAGCTGTGGGCTTGCGTTCTGGCCCCAATTCTTACTCTGTGGCTTTGATCTAGTTGCTTGGCCTCTCT
GTGCTTCCTTCTGCTCATCTGAAAACTGGAAATACCTAATAATGGTTTGTTCACACTCCCTCCCCCCTGAATTTTCCAC
CCCAGCCTGCTCTTTCTTTTCCACTTTCAGGAGCCTGGGTTTACATGACAACCTCAAAGATCCTTTCTGCGTCTTCTAC
AGAAATGACCTTCTTGAACTCTGTGGTGGTGGCGGTGGTGGTAGGGGGGCATGTACTAGTTGTGAGGTCTGTGGTGGCA
TTGATAGCCTCCGAAAAGCCAGCTTGCTGGCTTTCACCACTCCTGTTACTTCTCAAGCTTCCGGACTGAGAAGTGACAC
TTTGTATTCTTAGCTTATCTGTTCTCAGAGAGGTTAAATCATGGGTCATTTGTCCAAGGTTGCCCAGGGAGTAAGCCCT
CCATCTCCTTCTAAAAAAACCCAAAACAACCAAGAAGGCCAGGTAAAGGCACCTGGCAACAAGCCAAACATCCTGAGTT
CAATTCCCAGGATCCACAAGAGGGAAGAGAGGGCTGATTCCTATAAACTGTCTTCTGCTCTCCATGCCTGTACTGTGTG
GAGCACACGCACATGTGCAAGCATGCACACTCATGCATGCATGCATGCACAGACACACAGGCACACACAGAGACATACA
TACACATGCATGCACACACACTAAACAATAAAGTAATGGTTTTCTAAATTTGTTTTTTTTTTCGAGACAATGTTTCTTT
GTATAGCCTTGGCTGTCCTAGAACTAGCTCTGTAGACAAGGCTGGTCTTGTGTACTGGCTGGTTTTCTGTGTCAACTTG
ACACAGGCTGGAGTTACCACTGAGAAAGGAGCTTTAGCTGGAGAAGTGCCTCCATGAGATCCAACTGTGGGGCATTTTC
TCAGTTGTTGATCAAGGGGGGAGGGCCCCTTGTGGGTGGTAGTCCTGGGTTCTATAAGAGAGCAGGCTGAGCAAGCCAG
GGGAAGCAAGCCAGTAAAGAACATCCCTCCATGGCTTCTGCATCAGCTCCTGCTTTCTGACCTGCTTGAGTTCCAGTCC
TGACTTCCTTTGGTGATGAACAGCAGTGTGGAAGTGTAAGCCAAATAAACCCTTTCCTCCCCAACTTGCTTCTTGGTCA
TGATATTTGTGCAGGAATAGAAACCCTGACTAAGTCATCTTGAATTCACAGAGCCACCTGTTTCTGCCTCTATCTCCTG
AGTGTGCAGGGATTAAAGTTTGCCACCATGCCCGGCTGGCTTTTTTTTTTTTTAATGGTATGTGAATGATGAATTACCC
CAGCCCCATACTGGGTACTTAAGAAGGGTGCTCAATATGGAGCTGCGGTGATTGTTTAGCGGGTACAATGCTTGTTGGG
AACTCCAGCTCTCTCATAAAAACTGAGTATAGAGGCAACCTCAGCACTTAGGACAGAGACAGGTGGACCCCAGGGCTCC
AGCACCCATAGGGTGTCTCACAACTGTGTCACTACAGTTCCGGGGCAACAGACATCTTCTCCTGGCCTCCAAGAGTACC
AGACATACATGCAGTACCCACAAACATGCATACCTACAGGCGAAACACCCATACATAAAACATAAATAAATAAATAGTT
TGGGCTGGAGAGATGACTCAGCGGTTAAGAGCATTGACTGCTCTTCCAGAGGTCCTAAATTCAATTCCCAACAACCACA
TGGTGGCTCACAACCGTCTGTAATGGGATCTGATGCACTCTTCTGATGTGTGTCTGAAGACAGCTACAGTGTACTCATA
TAAAATAATAATAAATAACATTTTAATTAATTAATTAAAAGAGAAAGAGTGTTTGTGCTGGGTGTATGCCTTTGATCCC
AACACTTAGGAAGCAGAGGTATGCAAAGTGAGATCCTATCTCAGACAAACAAACAAACAAACAACAAAAACAAGGCAAA
ACAAAAACAAAAACAAACCCTCCCTCTTTATTGGAATATAGTTATATAACTCTACACACTTGCCAAAGGTCATCAAACT
GCACTAAAATGGTCAGATCTCATTATTTTCTACTTACTATTACATGCTTTTATATTAAATTCTACCGTATTACACATGC
AGCTTTAACACTGTCCAAACGTCCCACTAATGATCTGCCTGTCCCCTCTACTCTCCTCCTCTGTCACCTCCCCTCACCT
CTGACCTTCCAGGGACAAGCCCCCTGTGCTTCCCCCAGATCAATTTCTCTGTTGAAGGCCAGATTCCAAGTCACTTCCA
GTGCTATTCACTTTGAATATAGTCTGGGTTTGAGAAAATAGCTGCCTGTCTTGGTCAGCGTTCCTATTCCTGTACAAGA
CATCATAACCAAGAAGCAAGTTGGGGAGGAAAGGGCTTATTCAGCTTACACCTCCACATTGCTGTTCATCACCAAAGGA
AGTTAGGACAGGAACTCACACAGGGCAGGAACTTGGAGGTAGGAGCTAATGCAGAGGCTCCGGATGGGTGCTGCTTACT
GGCTTGCTTCCCCTGGCTTGCTCAGTTTGCTTTCTTATAGAACCCAAGACTACCAGCCCATGGATGGCACCACCCACAA
TGGGCTAGGCCATCTCCCATTGATCAGTTATTGAGAAAATGCCTTACAGCTAGATCTCATGGAGGCATTTCCTCAAGGG
AGGCTCCTTTTTCTGTGATAACTCCATCATGTGTCTAGTTGACACACAAAACCAGCCAGTACACTGCCAAAGGGTTTGA
ATATACAGGCTGGGGAGCTGGGGCCGTCATGTCTTCTGCACAGGGTTAGTCAAGGCCCAGACTTCACGTGGGCATCACT
GTTCACTGACCCTCTCTGGAAATATCTTGAATTCTCTACTAAGATGATACGTTTATTGGTTTGTTGGTTTATTTGAGAC
CAGGTTTCTCTGTTTAGTCCTGGCTCTCATGGAACTTACTCTGTAGACCAGGCTGGCCTTGAACGCAGAACCTTCTGAG
CGCAGGGATTAAAGGTCTGTGCCACCACTGCCCAGCAATGATAACTTCTTTACTCTAATTCATCTACTCCCTAGTTTCT
TTTTAGAGTGAGTCTTATTTTTTCCCACATATCCTCACACATATGGCTTCTTTCCTGCAGAGACCTGCAGTCTCTTCAC
TCAGTAGTGTGCTTCCCAGAAACCGTTTCTCTGAGTCTGTCAGGAAGGTGTGCTAACCACCCACAAAACACACCGGGAG
AACACCGGGAGAAACTCTGTACAAAGCCAGGCCTTATCTTGTCTCCCAAGAGACTCCAGCCCCAGCCCAGCCTGGATAA
TCAGCAGAACTCTTCCTAAGAGGTTGAAAGGGGCTCAGGAGCTGGCCACAGCCTGAGGAAAGCAAAGGGCCTCTTCACA
TGTTTGCTCCAACTGTTCCTACGTTTTATTGAAGAGAAATTTGCAGGCGGTGTCAGGAGTTCCCTGAGTTGCAGGTTCA
ACCCTGGGTTTCTAAATTTTTAGGCCTGAAGGACTCTTTCTTTCCAAGGTTATACCTTTGGCCTCAGAATTCTGGAGAC
GTGGAGCAGGTACCACACATCTCTGTAGTCCCAACAGACATGCCTCATCCCTGGGCCTGTCCTTGGCAGAGCGATGGGA
ACTAGCGATGGGAACTACCTCACACATGGAGGTAGTTCACGCTCTGAGGTAGTAGAACCAGCCCAACCACTCTGACTGA
GCTTGCCAGCTTTCTGTCCTGACAAGGCCTGGAATGAGCTAGCACCATGGGCAAAACCAAAAGGAGGCTGCCTGGGAGG
CTGGAGCGGGAGCAGCGCCTTCACGCAAGGCAGAGAACAGGAGGAACAAACTCTACCCCTGGGCCAAAGAAGACCTAAA
GGCCTCTCTGGACCGGAACATCCTGGAACGCAGGAAGCAGATAAAAGAGGCTCCCACTTTCTCTCCACTGTCGGGTGTC
CCCGGGGAGGACAAGGCCCTAACTCAGCAAAGCCTAGGACAATCTCAGGAAATGGAGGCGCCGGGTGCTAGGAAGGGGG
AAGGGGTGAAAAGAGGCTGCAAGCAGGAATGCTGATCAAGCTCTCCAGGGTGCCCTCCTTCCGGTGTGAAGAGGCAGAC
CCACATCTCAGCCATGTCGCTCCAGAGACACTGCGGCAGCGGCAGCGTGCTGAGGGCTGCCCAGCATGGCCCCTCTCCT
GCTTCGGCTTTCTCCCAGCTTGCTCTCACGTCTGTCAGCCCAGTTCCTTCCTACATCAGCTCAGTTTCCAGAAGATTCA
AAGCTCACTCCCTTCACCAAAAGCACAGATAAGAAATGACCTCCATGGTCTCCTCACTGTCCCCGAGGCACAGCCACGG
CCTTATTCTGTTTTTGTTTTGTGTTTTCTGAGAGACTCATTGCTATTGCTCTGACATGAAGCTCCCATTACTGCTAAAG
TCATCACACTTTTGTGTGTGCATACCCTCGCCTCTGTGTGTGTGTGTGTGTGTGTGTGTGTGTGTGTGTGTGTGTGTCT
GTCTGTCTGTCTGTCTGTCTGTCTGTCTGTCTATCCAATGGACCCCTAAGATACACTTGTCTCCCAGCCCCCAGTGGCC
TTTCCAGGCTTTTACATGGGTGCTGGGATGCCTAAACTCAAGCAACAATGCTTTTGTGTGGAGACTTTACCTCCTAAGC
CATCTCCCAGGCCCTAAATGCCTCCCTCTTAGAGAGGACCGGGCCGCCAAGAATCACATTTGAGGGAAACCGTAACCAT
GAAAGAGAGAGAGGCAAACAAACAGAAACAAGTGAACTCCAAGGAGACCAGGCAATGTGGACGCAGGCAGAATGAAGAA
TTTCTACTAGACTTTCAACTCCCAGAGGACTGAAGATGAGTTGAGAAACAAGAAGACAGTGTTAGGAAAAGGGGATCGA
ACAAGAACTATTCTAAAAGTTCAAAGTGATAGCGAAATACAGAAAGGTTGGAGTAAAAGAGAGAAAACTTCTAAGAAAA
GAAAGATACCAGGTCAGGAAAAAAAGACTTAAAAGTTAAGCAATCATTTTTTGGAAGGTTTGACATCCAAATGACTGAA
CTTTCAGAAAAGAAAAGGAAGCAACACAGCCAACCATATGTGGCGACTTGTACCTATACTGACCCTATCTATAATCCTG
TCACTATCAAGGCGGAGACAGGAGGATCTGTGAGCTCAAAGCTAGCCTAGTCTACATATTGAGTTTCAGGTCAGCTAAG
AGCTATATAATGAGACCCTGCCAGAGAGAGAGAAGGGGGGGGAAGGAGGAGGAGGAGGGGGGAAGAGGAGGAGGAGGAG
GAGGAGGAAGAGGAGGAGGAAGAGGAAAGGGAGGAGGAGGAGGAAGGGGAGGAGGAGGAGGAACCTGTATGAGGCCATC
CCAGAGAGCTTAGGGACATGAAATATGGGATGAAAGGGACCATTTAAGGACCAGAGCAAAGGATAAAAGCACCGGAAGA
CATGCCATACATATGCCAGGGCACCAGATGTGTAGAGAAATCCCCAAAAGCTTCAGGGAAGGAGAGAATGGTTTACACA
CAGGGATGTGCAGCCAGGATCCTGTTTTCCTCCGTGGCAGCAATGGGAGGAAGACAGTGAATAACTCTTTCTAAATATG
GGGGTTGGGGTAAAGATACCTTGGAGACAGAAGAGCTGGCTCAGCAATTCAGAACACTTGCTGAACTAGAAGAGAGGAC
TTGAATTCAGTTGCCAGTGCACACATGCACACATGAAGCAGCTCACAATCTGCCTGAATCAGCTCCAGGGGATCCAATG
CCCCTTTCTGCCTCTACAAGCACACACATGGCACACACACAAATAAATAAAAGTATTCTTATTTATTTATTTTATATGT
ATGGTTGTTGTGGTTTGAATAAGAATGACCCCCACAGACATGCATTTGAATGCTTAGGGAGTAATGTTTGAAGATATGG
CCTTGTTGGAGGAAGTGTATCACTACCTGTGGGCTTTGGGTTTAGTTTAGTTTTGTTTTTTCAAGAGCGACATTGTTTC
TCTGTGTAGCCCTGGCTTTCCTGAAACTGATTCTGTAGACTAGGCTGGCCTAGACCTCACAGAGACCTACCTGCCTGTT
TCTGCCTCCGAGTGCTGGGATTAAAGGAGGGCGCCTACACTGCCATCACGACCACCCATCTGGCTTTGGGGTTTCAAAT
GCCCAAGCCAGGTCCAGTGGCTCTCTCATGCTGTCTGCAGATCCAGATGTAGAACTCTCAGCTCTTTCTGCAGCATCAT
AACAGCCGATGTGCCGCCATGCTTCCCAGTGTGATGAGAATGGACTAAAACCCTAAACCTGCAAGCCAGCCCCAACTAA
ATGTTTTTGTTTGTAATTGTTGGGAGAGTCACGGTGTCTGTTGAGGGGAGTAGAAGAGGGGAGAAGAAGGGAGGAGGTT
TCCTGGAATTGGAGTTACACATGGTAATGAGCTGCCTTGTGCGCGCTGGAAATAGAATCCCAGTCTTCATGAATAGCAG
TCAAGATGACTTTAACTGCCAAGTAACCTCTCCGGTCACACTTCCTCCAACAAGGCCATACCTTCTATTGCTACTCCTT
AAGCATTCAAATGTGTGTCTGTGGGGACCATTCTTTATTTGTTTGTTTGTTTGGGTTTTTTTGTTTTGTTTTGTTTTTT
TGTTTTTCAAGACAGGGTTTCTCTGTATAGCCCTGGCTGTCCTGGAACTCACTTTGTAGACCAGGCTGGCCTCGAACTC
AGAAATCTGCCTGCCTCTACCTCCCAAGTGCTGGGATTAAAGGCGTGCGCCACCACCATCCGGCCTGTGGGGACCATTC
TTATTCAAACCACAACAACCATACACATTAAATAAACAAACAAACAAACAAACTTTGATTTGTGTGTGTGTGTGTGTGT
GTGTGTGTGTGTGTGTGTGTGTGTTTGGTTAAAGGTCCTCTGAATCAAGAACAGCTTCTTTAAAATTTGCTACCTGGGG
ACTGGAGAGATGGCTCAGTGGTTAAGAGCACTAGCTGCTATTCCAGAGGTCCTGAGTTCAATTCCCAGCAACCACATGG
TGGCTCATAACCATCTATACATAGATGGAGATCTAATGTCCTCTTCTGTCATGTAGCATACATGCAGGCAGAATCCTGT
GCCTTTAAATTTGCCCTCTGCAAGTCTGTTTTTAGGGATCCTACAAGACAGCAAACCGAGAAAGATGAAGGCAAAGAGT
CCAAAAAACACAGGACCAAAGAGAAGTAAATTAAGGGGAACCCTGAAATAACAATGAAGGGGGGATCTCAAGGAGAATA
CTGTCGGCTGGAGCCACTGTCTATGGTGTCCAGGGAGTGTGCCACCAAGACTGAGCAGGCCTGCCTTTGAAGTGGCAAA
GCTTTGAAACCTCAAAGCCTGCCTAGAGTGACATACTTCCTCCAGTAAGGCCATACCTCCTAAACATGCCCGAAGAGCA
CCACCAACTGGGGCTCAAATACTTAACACAGGAGCCTCTGGGGTCCACCCACATGCAGACCACCAGAGATGGAGATTCT
TTCAGCTCTGACGGTGCCTCTCAGGGGGCGCCCAGGACATATTTGAACAAATAAATGAGATCTGTGACAGAATAGATGC
CGGGAAGGGTGAACAGGGGAGTGGCTGTGTTTCATAAGTGTCAACCCTCTTTGTCTTGAAGGCTGTGCTCCTCTAAGGC
CACAGAAAACCCAGGAAAGGCTGAGAAGGGAGAAAAAGTCAAAAATCAAAATTGAAGCTATTAATCTGTTTTCTAAAAG
ATCTGGGTGCTGGGAATGGTGTCACACATTTAATCCCTGCACTCAGAGGCAGAAGCAGATGGATCTCTGTAAGTTCGAG
GCCAGATTGGGGTCTATGTAGTAAATTCCAGGCCAGCTGGAGCTACACAGTGAGAATGTCTTTAAAAAAAAAAAACCTG
GGAGATGTCTAGCCCTGAATGAAACCTTGTCCCAAACACTACTCTGTCACCAATACCTTACCCCAGATTTAAGACCCAC
CCTCCAAGCACTTACCCCAACAGCTGAGATGCCCAACACTAGGGGGCACTCTTCCATCACAGTGGCCCTGTCCTCACTC
CTCCTCTAGGGGAAAAAGTGGAGGCTCTTTTCTGTAAAGACTGACACCCCAGAAACTCCTCATGTTGGATGCAGCTTCT
TGTCACGGCTAGAGAGTCTTCCCAGACATCAATGGCTCATCCACTAAATCATTATCCACCATTCTTAGTTTCACAGCAA
GTAGAGGTGTCAGGGGTTAAATTCATCTGTCTTAGTTCAGGGGCCTGAAGGTGGCAGCTCTGGGCTCCCTTCCCCATCC
TCCGTGTGACAGCTATCTAGAGAAGAATAAACTAAAGGGCATCCTTCTGCAGTTTCTTCTTCTATGCCAACATCCATTT
GCCATGACTTCCATGTGGAACTAGGTTCATGGTTGGTCTGGCCACATGTACACCCACACGGGGAAGGAAATGGAGCAAT
GGGCAAAACTCCTCTCTCCTTACCATGGCCATGGATAACCCCACTCCTGCAGCATCACCAGACTGAAGACCCACAGAGG
ATGGCCAGAGGCTCGAGGAGTACACGCAGCCCGGAAAGTCCAGCCCCTTCGGTTGTAGAAAAGCCACTCCATACCTTCA
CAGACACGGGGCCTTAATTCACAAATGCTCCATCTTTTGAGGATTTAACCCCCGAGGACACAGCCCCTTCCCAGCTACC
CTATGTGAGCCTGCTGGTCTGAGACCTGGGAAGAAAAAGAGGAGGTCACAGTGATGGAAAGCTGGGGATCTAAAGCAGA
GGTAGGGGCCATTGAGGATGAGCAGAAAGTGTGCAGAAAAACAAAACAAAACAAAACAAAACAAAACAAAACAAAAACC
ACCAAAGGCTCTTGCTTGGCTCCTCCACCAGTGGAACAGAGAAGGGTTGGTATTTTCATCTCTGTGGCACTTACATCAT
CAGCTCTTCTAGTGTTTTGTTTCTGGCCCTGGATACTAGTCGGCTTCCTGTTCCCATAACCTGTCAAGTAGAGTGTCAA
GGGAAGCTGGGAAGGGGAATTTCACAACCAGCTGGTTTCCTTTGATGTTGCCACGAGAGCCCCAGAGTCCCTCCCCCCA
CCAACCAACCATTTGGGAAGTAAGTAAGCAGGTCTCTTCTGGGTAGCTCAAGGCAGGGGGCTCGGCAGAGGACCTGCCC
TCACATTCCAGCAGGGGGCTGGGAGGTGCAGCTGCCTGATCAGGATGAGGAGGGAGGAGAGGGTATGGGCATCTGAACA
ATTTGCTAAACCCAGAACACACCTCGGATGCAGCCACACCCCTGTGAGCTGAGCTCTCCAAAGCAATGGCTTTCTCTGA
GAACTGGGTCAGCTTTGCAGCTATCCCCGGGCAAGTTTCCAAACCCAGCAAGTCCCAGAGTCATCAGGGAAGAATCGTT
ACCTGCAGGGGGTGGGGAGAAATCAGGGAGAGAGAGACCGAGGTGAGGTAGGAAGCACCTGCCTCCCTACTTGGGTCTC
TTTCTCCCTGTTCTCAGTGAAGATGGATGTGCAAGCAGGCTGGCCACCCCAGGGCCCACCAGTGTTTAAGCTCCCCTGG
GACATTTGAGACAAGGCCACGGTCTCAATCCAGCACCCTGCAGAGCCAGGTGTGGTTGCCAGCTCAGGCAGATTCCACT
GAGGCCTCAGGCCTGACCTTCTTGCAAAGAGGAGAAAAAAAAATGTGTGTAAATTCCATCTGAGACTCCCTCCCCAGAC
TCTGTCCCTTTGAGCCACATACCTAAACTGACTCAGGGCTTGTTTTGGACTGAAGTAGGCTTGCCCACCCCCCCAAATT
GAACCTTTGGATTCTAGCTGCCTTAAACTGGCCCCTTTCTCAGAGTATCTCAGGAATGCTTGTTAGTCAAACATTTGTT
AGGTGTCAGTGAATCCCAGGACTGCGCCATATGGCCTTTCCCCTGATTTTGTAATGCAGAGCATATGTGCCTGCACCTA
GTAGGTGTCTCTCCACCAAGGGTTTCCAAGAATGCTGCCCATGAAGTGAGGGCACCTGGGAATCCCTTCTCTAGTCTTT
GTCATAGGCTCCTACCGCAGCCAGCGGGGACAATGGTGTGTGTGTGCGAGCCACAATTTCTCTTTGTAAGGTAGCCTTT
AGCTTGGCACATTTGTTATTTTCTCATCTGTGTCAGACACAAACAGAATCTAAAATGCAATATTTGGATTTCAAGTAAA
ACATATACACATTTTTTTGTTTTCTTTTTAGGCTTTTTGTTTTATCTTATATGTATGAATGCTTTGCTTGCATGTACGG
TCAAACACCATGTAGGTGCCTCGTACCAGCATAATTTAGAATTGCCTATCAGATCCTCTGGAACTGGAGTTACAGATGG
CTGTGAACCACCAGGTAGGTGCTGGGAATCGAAGGCACATCCTCTGTAAAGGCAGCAAGGGGGGTTAACTCCTGAGAAC
CCCTCCCTTTTAAAGGAAAGCTTTATACAAAAATAACCACATCCCTGACTATTGAGTTTGAAAGGTTTCTTGTCCTTGG
TCCCCTCCAGCAAACTTAGGAATTGAGAACGCCGAGGGAAGGAGGGGGTTGGGCCCGGCAGTCCCGCAGCAGAAATTGG
TCCTGATTCGAGGTGGATGGGACTGAGGAATGCACGCTCTAACCAGAAAGAGGGTGGGGGGGGCGGGGAGGGGAGGGAG
TCAGAGCAGCTAAGCTGGAGGAGTCTCCTGGGGAAGTAGAAGGGTCCAAATGACAAAGGAGTCGATTCCACACCAAAAG
CCAGAGAAGCGGAAGACAAAGAGGGTTGGCTAGGTGTAGACGAGGTGGAAAAGACACTGAAACTCGGGACAGGTAGGTG
GAAGCAGAGACTCTCTGCCTTGTCAGGTTACGGAGACCGGATTTTGAAGACCAAGAAAAGGGACCTGGCTTCATTGGCT
ACTCTCTGTGTGACCCTAGGCAAGTTGTTTAGACTCTCTGAACCTTGGTTTCTTATTTATTACAGACATGATAAGAGCA
CCGTCGGAGGGTTACTGGAGTCGGATGGCCGAGTAAAAAGCAAGTGGTGACCCGCCGCGTGCTTGGCTTAAAGATGAGC
GGAGTTAGGGAGGAGTTGGACCGAATGCCCAGTGAGTGGTTGATTCATTTAGTTAGGTAGTGGGTTAAATCCACCAGAT
TTTCAACCCAGATTCACACACTTCCTGTAGAGGCAGGATTAAAAGAGCTGGCCAAGGTGTTTGTAAACAATAAAAGTGC
AATTCAAAGTTTGCCGCTGGGGGGTGAAATAGTAGGAGTTAGGAAAGAGGCTGGGCAGCGGCGGGCCGAAACATCGTTT
CGTCTGCAACACTGTCAAGGCTGGGCGGATGTGAACTGCTGTGCAGTCCTCCCGCCATTTCCAAAACGCCACAGACCCT
TCTTACAGCCACCATAGGGGCGTGAGAGGCTGATCCCTGGAGTCGTCCCTCGGTGCCTGTAGGAATGGAGGGTGAGAAC
CCGGTGCGGGCAGTGGTGGGGCGATCTTGGGACGAGTTGGTTAAGACACTCCCAACAGCTAGGACACAAAGCAGGGTGG
CAGGGAATGAATGCCGCACGGCCGCCAGAGCCCAGAGTGGCAAGGGGACAAGGCTGAGGCCAGCCCAGCGGCCAGCCGC
AGCGCCGCCTCCCGGGAGGAAGGCGGGGGCTGGCCCGGGGAGCGGAGGGAGGCGGAGAGCGGAGGAGGGCGGCGCGCTG
AGAGGTTATTTGTGGTTCGCTTTGATCCCGAGGGGGAACCTGAAACTCTCACATTCCTGGCATAGCAGCCGCCTCCAGC
GCGGGCTGACCTAGAGGCTGCGCACTGGGGCGCGAGCAAGCAGATCATCGGCGCCACCGACTAGTGCGCACCTCCGTTG
CTGACTGTACCTAAGCCGAACCGGGTCTGCGTGCGCCGGCAGCCGCGGGCCGCAGCGGTAAGCAAGGTGCCGGGCACTC
CGGGCAGTGCGCGAGGCCGGCGGGGACGCTTCCCAGCAGCAGCGGGACACCGCGATCAGTCCGACCCTTTCTGGAGATC
GGAGCTGGGGTGGACGAGCCTCCCCGGGAGCGGGAGGGGAGACTGTGAACCCTTTCGAGGATCGATCCTGGGTGTCTAG
CCTAGACTGCTCGCGCTTTTGTGGGGGTGAGGACACCATTCTCCGACCCACTGGGGCTGGAGGAAGGACAGAGGATCAG
TGTTTGGGAGTGTGCGTCTCGCGTGCGGGGCAAGTGAAAAGCTGCGGTAGGCATATCTCGCTGAATTATGAGGTTACCG
TGGCTGCTTTGTCTGGATTGGGGATACAGGTGGCTTTTACCCTCAGCTAGCTCAAGAATTCCTCTTTGTCTTCCTTGGA
AAATCCTCTGCGGAGCCACGAACTGGCTCTCACTTAGAAAGAATGCAGCCTGCTGTCCAGTCCGGTGACGTCAGCGCTG
AAGTATTTGGAAAACAAAGAGGGTTAGGGAGAGAGGCGTACCCGCGATGCTTAAAGACCTCTCGCGGTTGCGGGTTTTG
CTCGCCCGGGGCGCTCGGGCAGCCACCGGTGCTAGTGGTCTGCCTCGGTAGAGGTGAGTAACGCCGGGACCCTGGACTG
GCCTGGTTGACTTGGAGACTAGCGTTGGTTCCTTTTGCTGGAGTGAGCCCAGGCCAAGGCCAGAGACCCGTGTTTGCTT
AGAAAGGTGGCTGGTTTTCCGAGTGCAGCGCTCCAGTGCAGCCCAGGTACAGCGCCCGTGTAAGGAAGTTGATTTTCAG
TAAGTGCTCTTATATAAGTATTTGAAAGAAAGATTGTGCATCTAGGGGATTCGATTGGGACGTTTGTGCGATGAAAGGC
CTCGGAAATGTTCGGTGCTCCGAGAGCCTGGCTCATAGTCGGCGCGCCTGGAATCTGCCGGGAAGGCTCAGCATTCCTT
CTAGCCGCTCCCGGATCCTTAGCGCGAGTCAGCCCAGACAGTGATTAACTTAGGAAATACCGCGTAGAAAAAGCCGCGT
GTGAGCAGACTCTTCAAGTCTTACACATTGCAGGGTGCAGTTGGGGGGGGGGGGGAGGATTAGTTGCTGGCTTGGGTAT
CGTCAGGGCTCATTACCTTTCTCCAGCTCTGCCAACATACCATTCCCAAACTGAGGTAGGACAGGGGGCTAGCATTAGT
ATGGCACAGAAAGGGGCCACCACCCCCTTCCTTTGTCTCCCCCCTCGCCTCATTACCCCATCAACTGGCATAGGTAGTT
GTTACTAATATCAACAGAGATGCAGAGGGATCATCCCCTGTGTGCCTCTAGCATACACGTAGGGAGGGGTTTTTCTAAG
TAGCAAATGTGTGTGCCAAGAAAAGAGGTTGGTGGTCATGGAAAATGTTTCCCTGGCTCAGTTACTGCAAGCTCCCCTC
TGCCTCCTCACCAATTTCCATGGGGCTTCTTGTGCTGCTCACCTTGCAGAGTGGCTGCTAAGGTCATCTGTGACAGGGA
GACTCAGATTTATTTTATCAGCAGCCTTTGAGCCCAGGGTCTGGCTCACGCGTGTTTATTCTAAAGAATCAATGGAATT
ATTTGCTTAGGGCTTCTTTCTGCTGGGCTACATTCTTTCTTTACCGCCTGCCAGTAAACTGGGAACCCTATTGCTAAGT
GGTTGAATTTGATGGACGCCTGTCCTACAGTTTGCCCATGAGATTGTTTGGGGTAATGGCTGACTGCCGGGGACTGTGA
TATGAGATAGCCTTCTGGTTTTCTGTTGTATATCTGTAAGATCAGCTAAATCTCCATAAATGTTACAATGATAAGCATT
TGCCAAGGATTTATAGCATACCAGGCGCTCAGCTTCTTACATTTCAACTCCAAGAGACACACTGTGTGTGTGTGTGTGT
GTGTGTGTGTGTGTGTGTGTACATATATATGTATGTATATACATATATGCATATATATATTTATATTAATGGCAAACTA
AGGATCAGAAGCTCAGTAACTAAACTTAATCAATAGATGGCCATATTAGCACTTGCATCTGGGCCTGTGTAAAACCACC
CACCTACCCACCCAGCCTCCTCCTTCATCCAGAGTGTTCCTGCAAGTGCCCCTCCCTCCTTTGTTTGTGCTTCCTTTGA
CTGCTATTCTTTCTGTCTCTTAGGTGCTCTGAGACCAGGTGCCTGCTGGCCATTGTCTAGGCAGGTGTGTGCAAGCCAG
AGAAGCATGAGGACACTGGAAGACTCCTCAGGGACAGTCCTGCACCGTCTCATCCAAGAGCAGCTGCGCTACGGCAACC
TGACAGAGACTCGCACTCTGCTGGCTATCCAGCAGCAGGCCCTGCGGGGTGGGGCTGGAGCTGGGGGCACGGGGAGCCC
CCAGGCTTCCTTGGAGATCGGAGCACCCGAGGACAGTCAGGTGCTGCAGCAAGCCACCAGGCAGGAGCCCCAGGGCCAG
GAGCATCAGGGTGGAGAGACCCACCTGGCAGAGAACAGGCTGTACCGGCTGTGCCCACAGCCCAGCAAAGGAGAAGAGT
TGCCCACCTATGAGGAGGCCAAAGCCCATTCGCAGTACTACGCAGCGCAGCAGGCAGGGTCCCGGCCGCATGTTGGGGA
CCGGGATCCTAGAGGAGGGGTGTCCGGAGGCGGCCGGCGACAGGATGAAGCTCTTCGAGAGCTGAGGCATGGCCATGTG
CGCTCCTTGAGTGAACGGCTTCTGCAACTGTCCCTGGAAAGAAACGGTGCTCGGGTCCCCAGCCACATGAGCTCTTCCC
ACAGCTTCCCTCAGCTGGCCCGCAGCCAGCAGGGCCCCCAACCCCGAGGGCCCCCAGCTGAGGGCCCAGAGCCCCGCGG
GCCACCACCTCAGTACCCACACGCTGTAATGGCTCAGGAGACTGCGGCTGTCACTGACCCAAGATACCGACCCCGAAGC
AGCCCACACTTCCAGCATGCCGAAGTCAGGTAACCCCAGATTTCTTTCTAGTTCGGTCTCCCAGGAGGAGGAGAACCCC
AAGATCATATATATCGTGGATCTCAACTGTCATGATCCCAATCCCAGGGCACCCCCACCAGTTCCTAGGGCTGTCTGAT
CTAGATCTACCCTCTCCTGTCTTGAGAAGGGAGAGCTGGGGGGGCTGGAGGGTAAGGGGATTTAGCTGATCCAGTCATG
GTAGCATGCCTGTAATGTTTAGGTGAGGCAGGAGGATCACACCACCAGCCTGGACAACGTAGCAAGACTGTCTAAAACA
AAAAGAAACTGACATCATTGCTTTAACTCAAAATATACATAGCATTGTCTATAGCATTCAAATAAGGAGGGCTCCCAGG
GATGTGAATGGGGTTCAAAGGTGAGCATTTCTTTTTTTCTAAGAAAGAACTAACTAAAGACAATAGGAATCCTTACCAA
ACTTCTCCCGAGTTAAGTAGGCCAAAGGTGGAACTTATTGAAGGTTTTTAAGAGAAGGTGGACATTTTTTGTTGTTGGC
TCATCATTGGGAGGTGTTGCTATTGAGGCTGAAAGAACTAGGTGGCTCGCTGGGCCACTTCTGTATAGTTACTGTCTGC
CAGTTAATTACTTACTAGCTTTTCCCTTGTGCTTATGGGCCTAGATGAGTGAGTGAGTGAGTGAGTGAGTGAGCTGCTT
TGGGGGGAAAGGGGGGGCTCAGGATGCTGTTGGACCAGAGTAGTGACTTGGCTTGACTTCCTGTGAAGTGAACTCTCGG
GGAGTGTGTGTGTGTGAACTCTCGGGGAGTGTGTGTGTGTGTGTGTGTGTGTGTGTGTGTGTGTATTGGAGGGCATGAT
ATGATTGGCTCTTAAGAGATCAATACTAGCCTTAGAGAATGGGTCATTGGAGCTAGGCTGGGAGCCCAGTTAGTATAAG
CAGGAAGCCAGATCATTGCAGGAGTCCATGTTCCTAGAAATGAGGGGCTGCCTTAGTCATCTTTATTGGCCCCTGTGCC
TTTTCCAGAGAAACAGGCACCATTGCTGTACCTCCTTTGGGGTGGGGTGAGGTGGGCTCCAAGGAACCTGGCTGGGGAC
CCTCTGGGTTCAAAGTCAGTCACCCTTGCTGAATCCTTATTTGGTCCCTCCATTTCCCACCAATTCGGTTTTCTTCACT
CTGGAAGAGAATAGCCGCCTTGGGCCTAGTAGGGGAGCCAGGGGAGCGGGGATGTGTGGGGGTGTGGGGGGGGTGGAGC
CTCGTGGGTTTACTCTAGCCTCCACTCCCCCAGACCCCCACCTCACCTCCCTTTTCAGGGCCGGTCAGGAAGAGTTTCT
TCTTTTGGACCAGCCCTATTGAACCCTGCCAGGAAAAGATAATATCCAGGCAAGGAAGTGGGCTGTTTTGCAGGAGAAC
AAAAAGATAGGGAGGAGGGCGGTGGGAAATGGAAGAACCAGGAGCAAGTGTGAACGGAAAGGAGTCGTCGCCATTCTTC
CCTGGAGCTCTCGATCATTCTCGAAGAATGTCAGTTGTGTCTGCCTGGCCTGTGGTAGGGAGGAGGGGGACAACAGTTA
TCTGAGGCAACCCTAAGAGTGACCACACTTGGCAGCAAGGGGGAGTTTAGTCAGTCTAGTGGCAGCCCTCTTCTCTGCA
CTCCCTGGAGCCTATAGGAGGAAAAAGGATGGGCAGGCTTTAGGGACTGAGTCAGGGGCTCAAAGAACAGGGGAGCGAG
CCATAGGCTAGAGGGGTGATTTTGGACACTGGGGATGGCAGAGGGGGCATGAGAGATCCAGAAGTGAAATCCAAACTTC
CAAGAGGCTTCTAGAACATTCTGAGTACAACTGAGAAGGCACTTGTGACACTTGGGGGTCACAAGGCCCCCAGAAAGAG
CCTCTGGCTACCTGGGCCTAACTGTTCTCTAAACATTAGAGTGTAAGGATGTGTTACACTCATGGCCAAGGGCCGAAAG
AACACAGTCCTGAACTCTGCTATGGAGCTAATCCAGGGGTTGCCCCTTAACTGAGGTGATCACCAACAGAGATGGAAGC
TAGACAGTCATGGGTGAACCTGCTGTTACCAGCCATTTGGTCAAGCCTGGCTTCTGTGGCTGGAGACCCTTGTGATTTC
GCCGACACAGGTGCCACTTGGCAGCTACAGCACTGGTCTGCTGAAGGCCTAGCAGCTGGAGCTGAAATCTGTTCCCCCT
GGCATCTGATTCCCTGCTCTAACCTTAGCTGGGGTCTTGGAAAAGAATTTCGGTAGTTGTGGGTAGCAAGACCCTTGAA
ACAAAGAGGAGAAAAAACTCAGGTGAGCCGTTTTAGCTGACATGGCTTACCTGCTAGAATCCACTAGTAAGTGCCCTTC
CTGGGGCCTGGGGACTCTGTGTGGCTTCCATCCCAAAAGATCCTTCTATCAATTCAAAAGGTCATGAGCGCGTGTTTAA
AGGTAATATTTAAGCTGTATTTGCTTAGGACGACCCAGGTTGCTTGAACAGAATGAGCATCTGGATACAGCGCCACCGC
CACCCTACCCCACACCCATCCTGCTCTCTGACTCTTGAGTCTTAGGCACATACCTTGTGGCACAGGCCCTCCCTCTGGT
GGCTGCCTAACACTGGGGAGGCTCAGTCACTGGCAGTAATAGTCAGAGTGCGCGTGCACACGTGCGTGCTGTGTGTCTC
GGGGCTCTGAGCTTTATTCACCTCTTCTTTCCCAGGATCCTGCAGGCCCAGGTACCACCGGTGTTCCTCCAGCAGCAGC
AGTACCAGTACCTGCCACAGCCCCAGGAGCACTCTCCACCCCTCCACCCGGCAGCTCTGGGCCATGGACCCCCAAGCTC
CTTTGGTCCACCTGCAGTGGAGGGACCACCCAGTGCCCAGGCCACCTTGGGCAGTGCCCACCTGGCCCAGATGGAGACT
GTACTGAGGGAGAATGCCAGGCTGCAGAGGGACAATGAGCGATTGCAGAGAGAGCTGGAGAGCACTTCAGAGAAGGCTG
GCCGCATAGAAAAGGTGGGGACTCCAAGGACACTCAGAGCGGTGGCCGGTGCAGAGGGAGGCAGAGGGGCAGGAAAGCC
TATCAGGCTACCTTTTGCACCATATTCTCCTACACACACCCCCAGACACACACACACACACACACACACACACACACAC
ACATCCCAAGCTGACTCCAGACAGCTGAGGACCGACCAAGAGGCCTGCCCTAATGCATGAGCAGTTGCTGGCCAGTAGT
GGTTAGAAAGGACTCATTCACTAGCTCCCTGCACACTTGTACCCTCACCCTGCACAGCCCCTCCTGTGGACACCTCCCA
CAGAGAGCCCTTTGTGCACCCACACAACAGCCAGTCTGTTCCAGGCCAACACTGGCCAAAGAGAAAGCCCCTCCTCTCA
GGGAAAAGAAAAAGCAACGCATTGGCAAAGCAAGCCAGCTAGGAGCACGTGGAAGACCTGTGGCTTCCAAGTGGGAGGG
GCAGCCACATGCTCTGGGCAGAGGCAGCTCAGGCTGGGGTCTGGCACCCCCACTTCCTCGGTGAAGCCTGGCCAATTGT
GTACTAAGGTTCAACTTTAGCAATAGCCTCAGGCTTTCCACTTGCTGTCTATCTACAGTGTTCTGGATAGGCTGAGCCA
CCTAGGTGCTCTCATCCCTGGTCCTTTGTAAACACTAGGCATGGGGCATGCTGGAGGTAGCCCCCTTGTAGAGCAGCTA
GCCCAGTGTCTAGGCAGCAGTCTGGTCTACCTAAGAGGCTGCAGTCCTGTGGCAATCTCAGTAAAGTCACTGGCCTCCC
TCCCTAGGACCCTTCCTGAAGTTGAATTAATTATCTGTCTCTTACAGCTGGAAAATGAAATCCAGCGGCTCTCTGAGGC
CCACGAGAGCCTGATGAGGACCTCTTCCAAGCGTGAGGCCCTGGAGAAGACCATGAGGAACAAGATGGACGGTGAGATG
AGACGGTTGCAGGACTTCAACCGAGACCTTAGAGGTAAGGACCAGGCACGAGGGTTGGGGTGTTGGGAGGACATCATCT
GTCAGACTTTGGGGGCTCTGTTTTCAGGGGGAAATTTAGAATCTGTTCTGAGCTATAGAGGCCTACATTAGCCCCCATC
GTTGGGCCCTGAGCTGAGCTGGGACTTCCTTATAATCCCATCTCCCCAGTTTAGAGGGTGCAAGCCTACAGTTAGGACC
CGAGGGTAAAGCCCAGGCTGATAGCATCCCTCTATATTTAAAACATCTTTTCTCCTTATTTGAAACAGAGAGATTGGAA
TCGGCAAACCGCCACCTGGCAAGCAAGACCCAGGAAGCCCAGGCGGGCAGTCAGGACATGGTGGCGAAACTGCTTGCCC
AGAGTGAGTAGGGGACCAGCTGAGAAACCTGGGTGGGTGTAAAGGCAATGAGGCACAATGACGTGGCCCCACTACCCCT
GCCACTCCCTTCCCCTTCAGGTTTAACCCACCCTCACTACCCCCACCCCCCTTGCAGAGTGGTACAGATTCACTGAATG
GCTGCTCTGGGAAGGGGTGATTTCCAAAGATGAATAGGGACTAGGGAGCCTTAACTGGCTGAGCCACACACTATGGACT
CTCAGTCTCTCCTGTAGGGAGGCCATGACAGCCCTGGCAATAGATAAATGGAGGATGATGAGGGTTCTTTGGAGTTTTC
CTAGGAATGACAGACAACAGAAAGGCTGGGGACATGGGAAGCTGCAGGGGATGGTCCACATGGTGAAACTTCCTGGGAC
AGCCACAGATGGGTTTAGTCAACAGCTACAGTCTGAAGGAGGCATTCAGAAAGTTTCTCTTTAAAACAGGGAGGTCCAG
GCATCCTGGAAGCTTTATAGACTAATCAATTTGTTCCTTCATTAGTGGAGCCAGGCTAAGCACCTGGCCAGCGCTGGGC
TGAATGTGAGAATCCATCCAGAGGATGACAGGGAGGTGAGATGAGCTTAAGGCAGGAGCAGGTACCCACAAGTTCAAGG
GAAAAAGTGTTCCTGAGCCATGTTCCTGGTGATTAGGAAGCCCCTGGTGACCCTGGGAAATCTGGGAACAGCAGAAGTA
TGGAGGCAGTGGAGGAGGTCTCAGACACTGAGGGGAAATGACAGGCTTGTAGGTAAGAGGTATTTAGGAGGAATATGTA
TGAGGGTACTTTGTTAGGGGTGTCTAAAGAGGGAAAGACTCCAGAGAGAGGGGGTTTCTGAATGCTAACCGCCCCACCC
CCTTCCTTCGGGGACTTAGAGTGCTTGCTAATAAATAAGCCTTTGGCAGGGAATGGGCTCAGATTGGGTAAGACCTGTG
CTATCCACTACTACCTTTCAAAATGAGGTGGTTAAAGGTCTCTGGAACGGAGGTGAAGGGGAAAGAGGTGTGTCGCCTA
GGGGTTTGCCCTTGGGAGTCTGTAGAAGCACCTACTGCTGTGGGAAGAGAGATTAGGGGTGCTTTGGTGGGAGGGGTGG
GTTTTGGAGCCCGGCTCTGACGGAGAAAGGAAGTCTTAAGGAGGGTTTCTGGAAGCTTGCAGAGGGGGAGGGGGGCAGA
AGCAGCCGGGAGGCGTTTCCTCTCCCCGGGGAGCAAGCTAGCGCCAGGCTGTTCTTGGCTTCTTGCTGGAGCCATTAAT
CTCGCAGCCGCGGGTGACCTGGATGCCTGGCATACCAGAGCTGCTGGGCTGTTCCGCTCCCAGCCCGTCCCCCCCTCCG
TTCCCTCCCCTGTGCGCCTGACAGTGGCGCCCTTCGGCCCCGGCGGCGCCCAGCCTGGCTTTGGGAGGAGAGAGTATGA
ATACAAAGGGAGGCTAGGACCTGAATGTGGAGAGCTTGCTAAGATAGGCTCAGCCTCCCACCCTCAACCAAGGTATCTT
AGAGATTGTACAAGGCATCTGGAAACAAAAAACACTGGATCCACAGCTGGAGGAACTAAGAAACCCCCCTAGCATTAGA
AGGCTGTTTTGCTTTGTTTGTTTTGTTTTTCTATATAATCGCCATTTCTAAAGGGCTGAGGTTGACACTTTTTTCTGGA
GGGTACAGAATTCCTCCCCATAACTGGCTTTCCCCAACAGCAGCCCCCATCTCTGCCTGAATTGTCTGATATGGGCCTT
CAGGAGCTGACTCTGAGGCCTGACAACTGGTCCTGCCTGCTTTTGGACAGGTGTTGCCTTGGCCTGGCACAGGCTACAG
AAGCATCTACCTCCTAGTACTGCTAGGTCTGGAGTGAGGCCTGGACATTCAGACACAGGCTGTAGTGCTAGTGGGTGAG
AGAGGATGCTGTTTACGGGTCTGAACCTGGCTTCTTCCCTCCACTCCACTCACCAAAGGCAAGCCAGCAGAGTATGACC
AAGTCATGTGTAAAGCATCATGGAACAGCCCCGTGTTTTCCCTCCACACCAAAGAGTGAGGCAAGCTAGGCCGATTGTA
GCCTTCAGAAGGGCAAAGGAGATAGCCTAGATTGCCCCTTAGTGCCCCTTAATGCTGTTCAACGAGTTGGTGAAGGGAG
GCCTCTGCATACTAGGGCCGTGTCCTAGCCACACATAGCCCAAAGAAAGTTAACATTTGTTCTGTGTTTCCAGCCACAT
TTTGAGAGTTCGGTAGCTGAGTGTAACCTTGGTAGTTACTGTTTTGGATAATGTAGAGCCATCCACTGTAGCAGAAAAA
TCTCACCAATATAGTTTTGTTCCAAACCCTGTTCCCTACGAACTCTCTAGAGAAATAAGCAGCCATTCTTGGTCGACTA
GCCCAAGACTCCCAAAGGGCGAACATCCAGGTTCTTTGTTGGCTTGGCCAGGCTAAATGGAGAGGTTCCTCTAATGTGC
AAAGTGCTCGGCAATTGCTGGAATCTTAGACACAAGTCTTTGGACCAGCATCCTTTTCAGTGTGATGCATGGTTTGAAT
TCTCTTATGATGTTGCATGTGGGCCCCCAGACCATGCAACAAAAGGGTCGGTGAACAGGAGAGGCTACCGTCCGCAGAC
TGTACTTAAGAGTTCATTTCGGCAAGCCTATAGGGAGAAGGACTGTGAGCAAAGCCTTATAGCCGGAGCCTCCCAGTCA
GCCATTTGGTTCCTATGTTTCTTCCTCTATAAATCAGGAGCCTCAAATTCCTCCTGGGGGATCAAGTGTGATAAGGTTA
AAAGTGCACGTCCTGCTTGCACAACGAATGACCTTGTCACAGTACCCTCCCTAATCTATAACAGGTTGGCACAGTGTCT
GCCTGAGCAGTCTAGTTATCATGAAGCTTAAGGAAAATATGTTTTGGTAATGGTCATGTCTGGCACAGTTCAAGCAGCT
GAAAGGCCACTTTGGTTTATTTGTTTGTTCCAGCAAGGGTCATGGGGGTCAGGCTCTGCCGTTTACTCTTACCTTAGTC
TGCATCTCGCCGAAGTGCAATATGTAACTCGCAGACGCGTCGAGGGGGAGAAGCAGTTGTCACAAGTAGGCAAAGGTGG
CCGCCATCACTGACCCTTTCCTCTTTAATGTCTCCTCGGACAGGCTATGAGCAGCAACAGGAACAGGAGAAGCTGGAGC
GGGAGATGGCACTGCTGCGTGGTGCCATCGAGGACCAGCGGCGACATGCTGAACTGCTGGAGCAGGCTCTGGGCAATGC
ACAAAGCCGTGCCGCCCGGGCTGAAGAGGAGCTACGCAAAAAGCAGGCCTATGTGGAGAAGGTGGAGCGGCTGCAGCAG
GCACTGGGGCAGTTGCAGGCTGCCTGTGAAAAGCGAGAGCAGTTGGAGCTGCGTCTGCGCACGCGCCTGGAGCAGGAAC
TCAAAGCCTTGCGTGCACAGCAGGTAAGCCCAGAAGACCTGTGCAGCTGGCAGTACCGGGCACAATTCAAAGAGTCCCT
CCCTGCTCTCTAGTCTTTGTCCTCTTTGAAAGATGATTGCTTCATGTTCCCCTTGGCGCTGCTTGGTCCCTCTCAGGTG
CTTTGTATAGCAAAGGCTCTGCCTCGAGCCCTACTGTGGCTAATAACAGAAATTTGGTACCTTAGTACCAGTCAGTAAT
AATGTTCGTGCAGAAGGCTTGACCACTCTAATCCATCTTCCCAAAAATAGGAAGGAAGATGTCTGAGTCTGTGCTTGGA
CATAAACATCTCTTGTGTTTACCAACCAAATAGAGTTCTGTCTCTACACACAACTTTACCCTGTCATAGAAGGGGGGCC
AAGACTTTGTCCCAATGTGATTGCCACTCAAACCCAATTCCCACTTCAGAGAATTGCTTTTTAGTCGTATGTGTCCCTA
GTCTCCTGACCACCAGAATTAAGGAGAGGATGTGAGGAGTTTTGCTATTCCCTATATCTCTGGGTGGAAGTAGGAAGGG
CTGTCTGTATGTGCTGTCCATGACTGAACAGAGGGATGCATGTGAGAGACTTCATGTGAGCCTGAGGAACAAAGTCCCC
CTCGTGCCAGCCTTTACCCTTGACCATGCTATTCCTTGCTCTACAGAGGCAGACAGGCACCCTCGCAGGTGGTGGCGGC
AGCCATGGTGGGTCCGCCGAGCTCAGTGCCCTGCGGCTGTCTGAACAGCTGCGGGAGAAGGAGGAACAGATCCTGGCTC
TAGAGGCGGACATGACCAAGTGGGAACAGAAGTATTTGGAAGAACGGGCTATGAGGCAGTTCGCCATGGACGCGGCTGC
CACTGCGGCTGCCCAGCGCGATACCACTCTCATCCGGCACTCCCCCCAGCCCTCGCCCAGCAGCAGTTTCAACGAGGGC
CTGCTGCCAGGCAACCACAGGCACCAGGAGATGGAGAGCAGGTACCACAGGGTCAGGCAGGGGTATCTGCGGTCAGGGG
TGGAACGGTGCTACCCAGTCTCTTCCCAGTGTCCTTTGTCCCTGGTAAATAGGCCCCACCCATAGCAGCTCTGTATGTT
TGACATTCCAGAAATTATAGACAATTGTTTCCCTGAGTGAATCCCTGGTATTCGCAGGCTGGAGGTGGGTGGGTACCTC
CTAAGCTCTGGCCACTTCTCCTTCTCTGTAAGAGATCTTCATCTGAGTCTTGCAATAATAGGTGGGCTTCCAAGGCCAA
GAGGTCCTGCCTGCCCCAGGGAGAGATGTCTGGGTTTCTTTGCAACTTCTGTGAGGTGGGTGGGCTGGAGGAGCTGAGC
CTGTGGGTCTGACAGTGTGACCTCCTCCCTGACAGATTGAAGGTGCTCCATGCTCAGATCCTAGAGAAGGATGCGGTGA
TCAAGGTCCTTCAGCAGCGCTCCAGGAAAGACCCTGGCAAGGCCACCCAGGGCACCCTACGGCCTGCCAAGTCGGTGCC
GTCCATCTTCGCGGCTGCAGTGGGAACTCAGGGCTGGCAAGGGCTCGTATCCAGTGAGCGGCAAACTGATGCACGGCCA
GCGGGAGGTATGAGGAGGCTTGGTGTGGGAGGGAAAGGGGTAGGAATTTTGGAATGCATATTACCAGACTCTCAGAATA
CACGCCCACTGAGACCAGGGCTTCCTGACCCCCTCTTCTAAGGGATCGGGTTTAGCCTTTGGCTCCCGTGACTGCAACC
TACATGGGTCCTGCTAGTAGAGTGGATGGATGCTGGATACAGATAGATCTAGGCAAGGGTGTTCCTGAGCAAATACTAA
GAAATCCTAGATTCCAGCTACAAAGAAGGGCCACAGAACATTGATGCCTATCCTGACCCCATTTATTCCTATTTTAAAA
GAGGAGTCTTGCTGGGCGTGGTGGCGCACGCCTTTAATCCCAGCACTCGGGAGGCAGAGGCAGGCGGATTTCTGAGTTC
GAGCCCAGCCTGGTCTACAAAGTGAGTTCCAGTACAGCCAGGGCTATACAGAAAAACCCTGTCTCGAAAAAAAACAAAA
AAACAAAAAAAAAGAGGAGTCTTTTCATTCTATTAGACAGCTTGCAAATTAAGAACTACCCAGTCCACCAAGTTTGCTT
GGGGTGTCCTCCATTGGGACCGTGTCTCCTACATCAAGCTGTACCCATCAGTGTCCATTCTTTCTTACAGACCGGGTCC
CAGCAGAGGAGCCTCCGGCCACAGCTCCTCTCCCTGCCCACACCAAACATGGCAGCAGAGACGGGAGCACCCAGACGGA
TGGCCCTGCAGACAACACCTCTGCCTGCTTGGCCTCAGAACCCGATGGCCTCCTGGGGTGCAACAGTAGCCAGAGGACA
CCCTCTCTGGGTAAGCACCCCACTTGGTTAGATGAGGGTTGGGAACATGGTAGAGGTGCAAACTGGAACGCATGGGCCC
TGTTTTCCCCACGTTTCCGCAAACTCATCCACTGTGGAAAGCCCCACCGTAGCGAACTGAGGGTTGTTGGAAGTTGTCT
CAATGGGAGGCAGGGCCATCGAGCCATTTCAGAAAGAAGGCAACGCAACAGGAAGCACTCTAACTCCTCCAGAGTTGCC
CTCACATCCTCCTGTCAGTCCAGGAGCTTGACAAGTGAACTCAATGGTGATCTTGGGTTGGGAAGCTATTTGGAAGAGC
CCAGAAAGGAGGAACTAAGCCTTTTCTCTTTCTACTCTTGTTTCACATTTGCCCTTGCATAGCTGCCTGTGTGTGCCCC
ATGATCTCAGAAAAACCACAGGCATGACAGGAACGCTACAGCCTGCTTCTAACCCTGGGAGCACCCTTCCACTGATCAT
TCAAGCTAGTTCAGAGTCTAAACACAGGCATGGGCTTCCTCGGCAGAGGGGAGGGGGCACAGCCTTGTGCTTAGTTGAT
CAAGGGAAGAGGAACCTAAGGGCTGAGGCAGCTTAGGGGAGGGGGTCTTGTTTAAAAAAGACCCTGTGTTGGGAGCTGA
GGAGAACAAGTGGATAGTACTGACAGCATACACTTGTCTTCTTGTTTTTTCTCCAGACTCTATAGCTGCAACCAGAGTC
CAGGATCTGTCAGACATGGTAGAAATACTGATCTGAAGGAAGGAGGCAGTGCCTTGGGGACCCCGAAGCCTGGCTCCCT
CCTGGCATTCCGGCAGTTGTCTTCAAATGCTGTGCCTCACATCCTCCGACCAGTGATGCCTTGGTGCATGGGGCTCAGA
GAGGAGCACCCCAGGGGAGCTAGCTAGAAAAGCCTGCACCCCCTAGAAGACTGCTTTTTGGCCTATATTTCTCCTATGA
CAATGAGGAAGGAAAAGCCAGACATCACCTGCAGCCGGAGGCCTGTTGCTGGGGCAGAAAGGAGCTGAGCCCCCTCCGT
CCTGCCTGAAGGAAACAGCGGATTCTGACGCCCAAGAATGGCTGGGCACCAGCCGAGCGGTCTGCAGGGATCGTTAGGC
CAGCTATGGTGTTCTGTGGCTAAGCATGGCTCCAGCCAACACTATCTGCTCTTCAGTCCCTGACTGACTTCCTCAGATC
ACTAAGACTTGAATCCTTGGGAGGTGGGGGACGATGGGAGTGTGAGTCTTTCGGGAGCCACCAGCATGTCCCCTTGGAG
TAGGTTAGAGGTCACTTGAGAGGCCCCGCAGAGCAGTGTGTTTTGTATATAATTCTTTTTCCTTGGGAACAGAAGACTA
AGCGTGCCGGTTCAGAGTCTCCTCCAGGGCTGTGCTGCCTGCCCCTGCTGCCACAGTTCTTGAAACATGTGACAGGTCT
ACCAGATGTTCCTCTGCCTGCAGTTCCTGGCTGTGGATTTTGATACTGGTTGGCTTTCCTCTGCTTTTTAAATCCCTGT
GTTTCGTGCCCATGGGATTCTTATCACCAGCCTTTGTGAATGGCTCCACAGCTCCTATCCCTGGGACATCCTGTCAGTT
TGGTTATAAACTGAGCCAGATGTGATGAGCCACCACAAGCGGACATCTGTCATGCCCAGCACCAGGTTCTCTTTGAAGA
AACTGGAGAAGTTGTTCTCATAAATGTATTCGTTCCCACCAGCAGGGACTGGGAGAGGGAGCTATGTTGTTAAAACGAA
TCAGTGAACTTAAAGAAAAGTCCTCATTATGGTGCCCCCACCACAGAATGGCACCCTTAGAGGCTGGCTCCTCAAGTTT
TGGTGCCCCAGCCCTGAAGCAGGGCCAGCTACTTGGAATTAGGTGAACTACCCCCACGTGGCAGACCACCAGCAGCAGT
GACTTTGACTTCTGGTCTTAAAGCAACCCCCATTCAGCTCAAAAAGCGATTGGTGGATTTCAGATCTTTTGTGTTTTTC
TCCTCTGGTTGTTTTGTTTGTTTTTCTTTCACTGTTTGAAGTTCTGTCTGTTGTTTGTTCCATTCCCATAGTTCTCTCT
GCATTTCCTTTTTAAACATTTGCATTTGCTGGGCAATTGCGTATTTTTTTAATTCCCTTTCCCCTATGTAAAGCTGAGA
AATACATCTGGTTCATGTACGTTGTTTACAAAGATAAAAGTTTTTAAAAAAAAAGGAGGGGGAAAAGAAGGCAAAAAAA
TATTGTGAAAGGAAAAAAAAAACAACTTAATATATTTTGGATTAATATTTGGTATTTCTTTTAAAGTATTTTTTTTGTG
CTGTGAACGTTTTCTGCCAAAGACCATGACGTGTGTCTGTGTGTTTAAGTTATCATGAATATTTAAAGTGTAAACATGG
CTGTTTTGTTACGCCACTCTGTACCAGGATTGCTGCTGCATTCCGCCAGGTATGACAGTATTTTAATAAAAAAAGAGCT
TATAAGGAGATGTCTGTCTGTCTGTCTATTGGTTGAGACACAACCTAGGTTTGAGCCTCTAAGTTGCCCTCAAACCAGA
AATTCTCCCACCTCAGCCTCCTTTCCTACGGTGGATTTACAGGTGTGTGCCACCACCCCCAGCTACCAGACTGGTCATC
TGCAAGGATGAAGAAAAAGAGTGTGAAGATTTTGAGGGGTCACCCAGGTCTACTCCCTCCAAGGTCACGTGACACGTCC
AACTAGAGATTGTGTAAAGTGTTATTAAGAACAGTTTAAACGACATGGCTGCCCCTGCCACCACACTCAGCCTATGGTG
ACACAGAGATCATCTGAGGGTAACCAGTTTTGATCTTGGCTGCTGTACAGTAGGCATAAGTCTGACACATTACCCCCTC
TACCCTCTCATTCAGTGCACACAGCCCTGTTAAAGGATGCACGTACCATGTGGACCCTCCCACTGGGTCCATGCACAGT
AGGTCAGAGAGTGTGATAAAGTCTTGGGCACCTGTCTTTAGCCTTCTGTTGTAATACCATCACCACCCTTCCCAAAGTT
CACAAAGTATATTTCTCCAACTCAAAAAGAAAAAGGAAAAACTACCCATTAATCTTGGCACTGAATCCAGTCAGAACGC
AAGTGGGCGGGCGTAAGGATGACCAGGAGACAGCAGACCACCAGATGGGCCATGAAGCCCATGACAGCAAAGTTAACCT
GTGACCAACCATGGGTGAAGTCACATAGGGACTCAGCAGCCACGTTGAGGAATGTCCAAGCTGGAGTTCCAAAGAGGGT
GTCTTTTGGCCAACCTCCCTTGTCATAGGAATGGAATTCAACAGGTGGGTAGAATTCAGAACACACAAAGACTTCACCA
GCAATCCTGCAAGTGACTATGGACTTTGCTGTCTGCCTTCTGTGTGACTTAGTCTATAGACAGTCACCTTTATATTTCC
TGTAGACCAAAAGAAGGAAGAAATGAATCCCGATGTGCCCAGAAAACCTCCACGTACGTGTGTGTACATCTGCACATGT
GTCCCCCAGAGGAAATGTAGGGGCAGCAATATTTTCTGCTTTCTACTCCTCCATGACCCCATGGGCTCCCTTGTGAGTT
TTCTCGGGGAAGAAACTAAACAGGTTATTCAGCGCTCTGCAATCCTCCCTTACTGAGCCAGGAAACAGAAACTGCAGCT
GGTGGAGTTGGAGAGGCCCTGAGAAGAAAGGTATTTGCCTCAGGTGTCTGGACACCTGCAAAGGGGTGGGGCCTGGGCT
AGAACCTGGGTGCTCTGCTAGGCCCTTTGGAAACCAAGTTGGTCAGAGGGGACAACCTAATCTGAGCCCCAACCTTCCT
GTCTTGTCCCCCTGTCTAGGATGACTAAATAGGGGGAGCTGTGGGGCTCTGGGGCTGGGGGTGGGGGTGATGAAGCAAA
CCGATCCAGGCACTGCATATGAGCTACTAGCCTGGCTTCTCTCCTCTCTCTCACCTTCTGGTTAATTATTAACTCTCTC
TTGGCATGAGCATGTGCCAGGCTCAAAGAGCTTGCTAACAATGTGCCAGAAGAGTGACTGTAAACAGATAGAAACCCAC
AGAAAACCAAAGTCTCTCCATGGGTCCCCAGGAACCCCAGCTGGACAGGAAACTTGTTTTCAGGGCTTCCACCTTGTCC
CTTAGCCCAAGTGAAGATCTAACGGACACCCAGTGTCTTCTGAGACCTTGCTGGGTGAGCAAGTCTGCTGGAGGAAGTC
CAGAGACCTCACAGCAGCAATGCCTTTTTGGACCGACTATATCTGGGTCTAACCCCATGCAGAGTACCACAGCAACTAA
GTGTTCAGTATCCATTGGAAATGGGAAGACTTCATGCACCATGAAATCCCCCTTTGGACGTGCATACTTCCTTCACATC
TTATATCACAGAATTTACCTTAAGCCAGAGTGGTCAAGCATGGGTCAGCTGATCTGCTTTCCACATGGCCTGTTAGTCC
CCAGGATGACTTCTTGAACTTTCTGTAGCTGACAGGATTCCTCCCTACATCCTAGAGCCAGCAAATCTGGAGCCCACTT
CCACCAGGCTGCAAAGGTCACTGTCTGCTCTAAGTTGGAGCCCTCTACCGCTAGAGCCTCCCATCATGAATCCTAAGCC
TGCTATGGGGCATAAGTTCTTCCCAAAGTATTGGGTCTACAGAGAGTCGCCTTTAAAAAAAAAAAAACTGAATCTTATT
TAATTGAATTTCTAAGAAAAATGATGCCAAAGGTTGAATCTGATTTTTGTTAAGGGACATGTTCATTATTGACTTCACA
TTAACTCTACTTAGGGGCACCGGGCATCGTGCCTGCCCAAAGAGATGATGTTTTAGGAAACGTCCGTGGCTGGGGCTTA
CCTCTGTGTCTTCTAGGTGGGGCTTGGAACTGGAGATGGAGTAAGTGGAGGTCTATGGCACTATCAATGTAGAAGCCTG
GACAGATTGGCCAACCTAGCAAGACCTGAATCTACTTCTGACCTATGTCTTGGAACTCCTTTGTGCCACAAAGCAAACC
TCTAGTCTCCAGCAGCAGGATGGAGCCCATGTGACATTGTTGAGGGGACGTGATTCTCCTCTACTCCAGCAGTCTACCA
GTTTTGAATTTAGAGCAAGGCCAGTGTCATAATCTGTCCTCAGATAGGTGTCTTTTGCAAGCCCAAATGCTCCAACCTA
AGGACACATTCCAGGTATATTAGGCAACCTTGTGATCATTTCCCTGTCTGGACTGACCCTTCCTGAGTCTTTCTGGCCC
ATGACAATGACATTTCTAGCTGAGGGAGGGAGGCCCCTGTCCAGACCCTCCTCTGTAACTTCTGCTTGTTTAACACAGT
GTACTGTGGCAATCCTCTCAACAGATTGCATCAGCAGCAGCCATGCAAGCTTCTCCGTGGTACAGCAGCAGCTGTAAAG
CCACTGCCAAAGCTGGCCCTGAGAACTATGGAGACCTTTATTTGTGTGTGTGTGTGTGTGTGTGTGTGTGTGTGTGTGT
GTGTGTGTGTGTATACAGGTACACATGCCCTTGTGAAGAGGCCAGAGGAGGATATTGTGTCTCCTGCTTTACCAATGTT
TCATCCCCTTGAGACAGGGTATCTCACGGAACCTGGTGCTAAGCTGTCAGCGAGCTGTCGGAGGAAACCGTCGGCAAGC
TCTATCTATCCTCCTGTCTCTGCACCCCACAGGACTGAGGTTACAGGCTTGCTATGGTCACGCCCATGGAGTCTAGGAA
TCCAAACTCAAAATCTCACATCTGCACAGCACAAGAGCCTTCCTTCCTAAGGTGTGGGCTCTCTGTTTTTGTGTGGCTG
ACTTCGCTTCCCTCCATCCTATGTTTCTAAAGTGGCTATGGTGTCTCTTCCTTTCTTCCTTTCACCATTCACTGACTCA
TTCATTCATTCATTCATTCAATAACAGATGTTTACTGAGGCAGACCAGTCAATGTAAAGCGAATTCTGTTTCTGAACAC
TTTTATGTCCCAACTGTCTATATGCTCGTTGTATGCTCTCCCTAAGCCAAGCTCAGAAGTGCTTTCTGAATGAGTAGTC
TAAACAGGAACCGTCACACATCTGTTTCTCTGGCTCCAGCTTTGATCAGGCATCAGACCCCTCCACACCTCCAGCATCG
AAATAATACTTGGTGGGTTCGGCTCCAAGCATCTCACGTGTCATTTCTGTGTCTAGGATGCTTTCCCCATGGATAGCAT
CTCCCACTAGCAGCATCTATTGTTTTCTTCCTCCCTTCTTTCATGGTCCCACAGGAGCAGAAGACAGAAAAGAGCTGCA
TTCCCGGGCTGAGGGACCTCTTGTCAGGAGGGACATTCTTTAGGGCTCTGCTTGGTTGAGAAGGAGCCTCAGGTGAGAT
GACACAACAGTGCAGAGGCATGCTCCCTGTTTGCACAGTCACGTGTGGCCCTGAGCAAGATCCAGACTATAGTCCCTGG
GGATCTTTTGACCAGAAGTCTCAAGCCCCTCCCCATTCACCAGACCCCCTGTCATTCCCTTGAGGTGAGAGGATGACAG
GGACTTTGTGGGTGAAGTCCTCCAGGTTAGAGGAGGAGAAGCTGAGCCCCAGGGCCTGGGCTGAGCCATAAGGAGTGCA
TGAACAGGCAGCTGTGGGTGTGTGAGCCCAGAACTGGCCACACAGGATGGCTGGCCCCTGAGTCAAGCCTAGGCACTGA
CAGCCATGTGAGCCTATGCCAGCACAGCCGTGGGAACAGCAGGTCCCTCCACCCCTAAGGAAGCTGCTCTTGCCTGGAG
CCATTCCTGCCTGGCTCTGCCAGCCCTGGGATCCCTTTAGATGCATGCCAGAAGTGGGGTGGGTACAAAGGCTCTTTCT
CAGGTTTATCTTCTGGTACTAAGGCCCCAAATTCAGATTAAGATAAAGGCATCAATATGTCAGGCACATGGGCTCCCGA
GATCATCCTGTAGTCTTCTGGTGACAAGCTCCTTGCCCTGCCTCCCTGGAGACAGAACCTCTGAAATCAAGGTTCAGGG
AGGAGGATGGGTTGCTTCTGGGGCTAGCCTCGAATTAGCTATTTTGCCAAGGGAGTAACCTTGACCTTTTGACCTTCTC
ACCTCCACCTCTGAAATGCTGGAACTATAGGCATATACCATGACGCTCCTTTGGGGACATTCAGGGTTGAACCCCAGAC
TTCGTACACATACATATTCTTCTTAAAGCAAATCTGATGGGTGTGTGTTGTATATATGGGTACCACATTTGTGCATTGC
CCATAGAGGGCAGATTAAGAGGTCAGCTCGTAGATATGGAGACAGCTGTGAGTTTCCATGTGGTTGCCTGCAACCAAAA
CAGTATCCTCTGCAAGAGAAGCCATGCCCTGTATGACAGACATCTCTCTGGCCCTCACACGCATGTTCTTATGAACTTG
CATCTACATCTACAACAGACATACAAATGAGTCCCGAAACTACAGACTCATCTGTGCTCTGAGCAAAGGCCAGCCTCTT
TGGTGAGTGGCAAGAGTGCGGATGAGATGAAGTCCACGTGTCTGTCCAAGTCGCATCCCCAACACAGCTCTGAAGGAGC
AGGCAGCAAGCGAGCCTATCCCCTCCCTCTCTACCAGCTGCAGAGAGCACAACACCAAGCAGGGTCAGGTTAGACAGGA
ATAACCTCAGAACTCCTGGGTCCTGACTCAACAGCAGAAGCAGACAGCCATACGCATTCTTGACAAGTAGGTCACCCTG
GCCAGCCCACATGCTGGGACCTAAGATCTACTGGAAAAGATTTGGGAAAAACCCAGGTGTTGCAGACAGCCCAGAGTTT
GGTTGGTTGGTGTCTGGTGCACTGGTACTGGGAAACATGTTTCACAACACCACTGGGTATTGTCACTGGGCCCAGAAAG
CAGCTGAGGAGGGGCTGCAGTATGGAGGAGCAAGAAAACCCAACCTAGCTGTGTACTTCCTGAGCCCATCTGCCTGTTG
GGTGCTGTGCTGGGTGTTCTGCTGGGACACAGCCCTATAACTTGACTTTTCTTGCTTCCCTTTGACAGACGTGGAGACT
GAGGCTACATGATAGCAATAAATTCCTCGGGGCTGGGGCTTCTTAAACTTTTCACACACATATACAATAAATAAATAAA
TGAAAATTTTAAAAGTTAATAATAGGGAGACAGGTTCCCTGAGTCCCTCTAATAAATTGGATGACTAATCTGAATTCTG
GGGCCTGACTGGAGGGGCCAGTGGGAAAATATATGAAGATCCCTTAGCATGACATTTGGGATAGAGCAAATGTCCTTGG
AGACATTGTCATTATTACTATTTTACTATTTTTTTCTATCATTATTCTAGAAGAAAGTCTAGTAGACTCTCTTGCAGCT
ACATTCTATTACTAACTTTCCAATCTAAACTTGGTATGGTGGTGCATATCTATGATCTCAGCACCGGAAGGTAAGTAAA
GGTAGGAAGGTAAATTCAAGGTCATTTTCAGCTACGTAGCAAGTTTGATGCCAGCCTGGGTGACATGAGACCTCCCACG
AAGCAGCTGGCATGCCACAGAGCAGCTGGAAGGGCTCCCCTTTACCTCCCACCAGACATACATACGAGACTATTTTAGA
CAAGTGGGTAAAAAGCTCTCCACCATCTCCCTTAGGGACAAGGAAAGCCACTCTGCCTCTCCATCCTTTGTCCCCAGCA
TTTCAAGCTGCCCTTTGACTCTCATAGTAACATTGCAGTTACAACACAAAGGCAGTGAGTGACACAGAAGAACACCAGA
GACAGCCAGGGCCAAAGCCCTGCCTGACAGCTCTAGAGTGGACCCCGTGCAATGTGAAGTCTCTGGGCTCTGGTTTCAG
TACTCTAGGCCCTGTGTCTTCCACACCAGCCTTCCCAGAGCTGGGCAATGAGTCCACCTGGAATGTGTAACTGCCACGC
TCGTCGCTGCACTTAAAGAATTGGCCTCTGCCTTCCCTGCCAAAGCAAAGATTCAGATCAGCAGATCCAGCAAGGCCAG
ACATTGACTGGGAATGGTCCCCTCAAGTCAGGACCAGCGGTGCCTCCTCTTGGGTGAGACCCGAGTGTTTTTTTTTGTT
TTTGTTTTTGTTTTGAGGACCAAATATCCATCACTTGACAGAGCCTTTATTAATGGTGATTTCATCGTGGTCCCAGTCA
TAGGTGACCTTGAGGGAATTGAATAGAAGGAAAACAAGAGACTTGGGGGACTTTGAAAGTGTTGGGACAGTAAATGACA
CAGGTCTGGAGAGTGAGGAAGCCACAACAGGATGCTCTAAGAAAACCTTTCTTTCTTTCTCTTAAAAGATTTCTTTTAT
TATTTACGTATATGTATGTGTGGGTTCCCTAGAGGGCCAGAAGAAGGTGTCCAATGTCATGGAACAAATGTGATAGAGT
ATTGTGAACAGCTTGATGTAGGTGCTAGGAACAGAACTCAGGTCCTCTGTAAGAGCAGTGGTTAGTAAGCACGGGGCCT
TTCTCTCTAGTCCTAGCAAACTTTTACAGCTAGAGAAGGTGTGGGGGCTGCCCATTCCAGGATGTTCATTCCAGCATTG
TTTAGTAACAGTGAATAATTGAAAATAACACCACGGTCTAAGCACAGGGGACAGATGAAGAAATTATGGTTTAGCCATG
CACAGGTGAAGCTGGTGGTTCACAGGTTGGTTGTCTGGCTTTTAAAGGTACATTTGGTTTCTTGCAGAGAGAGAGAGAG
AGAGAGAGAGAGAGAGAGAGAGAGAGAGAAAGAGAGAAACAGGAAGAAACCGCTACCTCTGTGAGGAGGCTGTTCCAGG
TTGTCAAATTCTCTGCCTCACCGTGGTTACTCACCACGGTCAGTCTCTATCATCACTGGGTGCAGACCTGTGCATGCTA
TTCTCTGGATCTGGAACGTTCTCCCTGCCCCTTTCCTAGCCCTCTGCCCTGGTAAGTTCTTCTGACCCCTTAGGAGTTA
GCATTAACATTATCCCTCTAGGGAAGGCTTTTTGACTGTCCCACACTGAGTTAGGTCACCCTGTTTCATGTTCTCATTG
TATCCCTGTAAAACTTCATTAGTGCCATTTAGGAGAGCATTTGTGACAACTCTCCCTCGCTTAGGCATTCCACCTAATG
AAGGCAGAGGAACGTGGAAGATCTAAAGTCACCGTCAGAAGCAGACCTCCAGGACCCGTGTCACCCTCGGATACCAGAT
CCGATGAATGAAAAGCTTATGAAGCAGTGGTATTCCCATAGTCTAGAAATGATCTTCCTTGGCTAGAGAGATGACAGGG
TGGGTGAAGGGGCTTGCTGCACAAACATAAAGACCTGAGTTCGGATCCCAGAACCCAGGGAAAGGTAGGCTATGAGCTT
AGAGCTAGGGAGAAGCTACGAGGATGTGAAGTTAGAGGGATCTCTGGGGCTCACTCGCCACCAGCTTAGTACTAGCTCA
ATGGTTCTCAGCCTTCCTAATGCTGAACTCCTTTAGTGCAGTTCCTCCTGCTGTTGTGATTCCCCCAACGACAAAATTA
CTACTTCCTAGCTGTAATGTTGCCACACTTATGAGTTGTAATGTAAATATCTGATATGCAAGGTATCTGGTATGTGAAC
CCCTGGGACCCACAGATTGAGAACCACTGTTCTAGGTTCAGGGAGAGTCCCTTCCCCAAGGGAACAAGGTGACAAATTA
GAGCAGAATACTCAGAGTTCCCTCTGGACTCCAACTTCCACAAAGATGCACGTGCCTATGATCAGTTGAATACTTCTCC
CTACCCCCCCCCCCAAATGTATTGTTAAAGACACTATCTCACAATGTTTCTCTGGCTGGCTTAGAACTTACTATGTTAA
CTGGGTAGACCTTGAACTCATGGAGATCCTCCTACCTATGCCTCCTATGTGCTGGGATTAAATGAGTGTGTCACCAAGA
CTGGCCCCCAAAACATTTTTTTAATTACAAAGGAATAAAATTGTAACTTCAGAATGTGTGGGGCAGTGGGCATGGGGGA
ATAAAGAGAGTGAGAGAAAAACGGCGTCCGGCCAGAGTTCTGGTGCTCTGGGCAGGTGGACTCAGGAGGACTGCCAGGG
TGGGTGTCTGGCTGTGGGAAGCCACAGAACTCCAGCCTGCAGTGGGGTGGACAAGGGGCGTCTCACAGAGGCCAGAGAT
AACAGGTTCTCAGTCTCGGGCGTCCCAGACACCGCTGGACACGGTGGAAGAGCTGAGAACAAAGTGGGGCCGGTCATCC
CCAGGGCCGAAGGGAGGACAGGGCAGGGGGAGAGGGGGCTCTGGGGGGTTCTCACATGGGTGAGAGTCTTTGGTCTGGT
CTGTGGCTGGAGTGCAGGAAGGCCTTCCAGCGGGAGGTTAGATGTGGCTCTTTAGAGGAAAGCTTATTCCATCGTTCAA
GCACAGTGGGCCTTGATGAGCAGAGACAGTCTATGGTTTTAGAGCTTTATTGTAGAGAGGCATGGAGAAAGAGAGGAGG
TAGAAAGAGAGAGGCTGGCCATGGCCATGTGGAAAGAGGGGGGAAGGGAGAGAGAGAAGGAGGACTAGAGATGAGAGTA
AGAAAGGTGAGAGCTTAAGGAGAAAGAGGGGGAGCCAAGCAGCTCCTTTTATAGTGGGCTGGACTATCTGGTTATTGTC
AGGTAACTGTGGGGAGGAGTCTAGCCAGAATGTCAGGAGCTTGGGGGCTTTATCTGTGTGACTGATAGTCACAGAATTA
TGGAGTTGGGGACCCTGTCGTATCAGGCACCTGTCTCTGGGGATGTGGCTCACTGTTCCTTCCCTTGTAGAGTTTTCTA
CTGAGTCTCCGGAGCAAGTCCCATTCAACCAAAACAGGCTGCCTTTCACAGCCCCACAAGAGTGGAGAAATCTTAAGCC
ATGTGACCAAGGTAAACATCACCAGTCATTGGACATAGTGCCATCATGAGCCCTGTATAGAATGTACCAAGAAGGGCAG
GTCACATCTGTGGCTCCCTTCCCACCAAGGCACAAGATGCATCTCCATATGGGAAAATATCACACAATTGCAAATTCTC
TGTTGTTTTGCTTGAGACAGGGCCATCTTATATAACCCAGGCATATAAGAGCTCATGATCTTTCTGCCTCTATCTCTCC
AGTGTGCTAGGGCTACACCGGCTAGGACTTTAAAAACAAGAACCTTTCTATTCCATTTATGATTGGGGTGGGGGTATAA
CTTTCAGGAGTTGGTTCTTCTCCTTCTACCATGTGAGCTCCAGGACTCGAACTCAGGTTGCCAGGCTTAGTGTGGCAAA
TGCTTTACCCATTGAACTATCTCCCAACCTTAGACCACTACTCTTAAAAAAAAAAGTCTTAAGGCAGCTGGATGTAATG
GTACATGTCTTTAGTTGTAGCAGTCAGGAGACTGAGGCAGGTGGACTTCTGAGAGTTTCAGGCCAGCCAGTTCAAAAGA
AAAAAAAAAGTTAGGGAGAGGTGGTTTAGTGATTAAGCGCACCTGCTGCTCTAGCAGAGCACCTCCATGGTGGCTCACA
ACTGTCTGTAACTCTAGCCCCAGGGGATCCTCATCTATGGGCACCAGGCATGCACATGGTAAAGACATACATGGAGGCA
AATCACACATACACATAAAAATAGATAACAGGTGTGCGCCACCACCGCCAGGCAGATTCTTAGTGAAAAGCTGTTGTGT
GCAAGGATTTGCTTCATGTCTATGTCACCAGATAGCCTATTAGCTTCATGGAGTCCGAGGCCACAAATCCTGTCTTGTC
ATTGTTATCTCCTGACGTGATGTACCCAGTAAATATGTGCTGATAAACAGATACAGTTACGGACATTGGGGACATGGGG
AGTCATGGAAGGATGTGGAGGGAATGGGGGAACATGGGAGACATGGGGGATCGTTGGGGGACATGGGGGCATGGGGGAT
GTCCATGATGCATTAACTGACAAAATAGGACATGTGAGGCCAGTTTTATAGCTTGGTTGGCATGACATCCTGGGCTCAT
TCATTCTCCAGCACTGGAGGCAGGAGGATCAGAAGTTCAAGATTACCCTCAGCCACACCAAGGTCAAGGCCGGTCTGGG
TTTTGTGAGATGGTGTCTGAAAACAAAGAAAATTTAAACAACCAAAACCTATGACACGAAATAACTCATATGATCTGGG
GATAAGAGCACACCCCTGTAATCTTAGTAATTAAGAGGCAGGAAGATTGCCAAAAGTTGAGACTAGTCTGGTCTATCCA
GTGAGTTCCAGGCCAATCAGAGCCACAAAGCAAGACCAGTCTCAAACGCAATTGCTTAATTAATTACAATTTAAAAGTA
AACAAAAGCAAAAATACCTATGCCAATGTTTGCTTAAATAAAAAGAACATAAAGAAGAGGGGTGGTGGTCTCATTCACC
TTTAATCTCAGCACTCAGGAGGCAGAGGCAGGGAAATCTCTGTGAGTTTGAGGCCAGCCTGGTCTACAGAACAAGTTCT
AGAACAGCCAAGGCTACACTGAGAAACCCTATCTCAAAAAGAAAAAAAAAACCATAATGATTCAGAAATAAACTGGGGA
CAGTGGCTGTCACTGAAAGAAATAGTCAAAGGTGATCTTTATTTTGGTCTTAAAAAACAAAACAAAGGGCTGGTGAGAT
GGCTCAGTGGGTAAGAGCACCCGACTGCTCTTCCGAAGGTCCAGAGTTCAAATCCCAGCAACCACATGGTGGCTTACAA
CCATCTGTAACGAGATCTGATTCCCTCTTCTGGAGTGTCTGAAGACAGCTACAATGTACTTACATATAATAAATAAATA
AATCTTTAAAAAAAAAAAAAAAAAAAAACAAAACAAAACAAACCCTTTGTGCTGCACCTGGGTCCCTACACACAGACCT
GGAGCCAGTCCCTGTGGGGCGGGCAGCCGATGCAAGAGTATGAGTTCGGCAAACTGGTACCAGGATGCAGGATAGCCAC
AGGCAGCGTAGGGATACAGTACTGAATGGAGGCAGTAATCGCCCTCCATTTCTCAGCTCTGTGATCCAGGCAGCTTATG
TTTCCTGAGCACGTCTTCTAAACTGGCACATCCATTCCCAGGAGGTCGGAACTCATGGCCATTTGTTGTGTGGCTCACA
GGAGTTTTTCCTCTCACAGTTCTGGAGCCTTCTGAGGCTAAGATGTAGTCCAGGGTACACCACTCTGAATGTGTTGTGG
GAGACGTCTTCTTGCCTCTTCTGAGCTTCTAGCACTGTGGCTCTCAACCTTCCTAATGCTGTGACCCTTTAACACAGTT
CCTCATGCTGTAGTGACCCCCGAACCATAAGATTAGTTTTGTTGCTATTGCATAACTGTAATTTGTTCCTGTTATTCCT
GAATTATAATATTAATATGTGTCTTCCCATGGTTTTAGGAGACCCGTGTGAAAGGATCAAACTCCAAGAGATCTCAACC
CACAGGTTGAGAAACATTGCTCTAGAAGCTCTTGGCAACTTTAGTATTCCTCTCTTATAACTGAAACACGTCCATCCCT
ACCTCTGTGTCTCTAGATCTAAATCTTCTTCATATGCTCTTTCTAATCCAGTGTGGCCATATCCGATTCCTTCTTCTTC
CTCTCCTCCTCTTCCTCTTCTTCTTGAGTCTCACATAGCCTAGGCCTTGAACTGACTATCCTGTCAAAGATGACCTTTG
AACTTTTGCTCCTCAGATCTCCACCCCCTGAACCTTGGACTTACAGGTACATTGCACCATGCTGAGTTTATGTGGTGCT
GGGGACAGAGGCCAGGGCTTTGTGCATACTGGGCACAATGCTCTACAAACTTAGGGACGACCACCGCTCAGATGTCATC
TGATTTTGATTATACCTTTGTTTTGGTTTGGTTTTTTGTTGGTTTTGTTTTGTTTGCTTGTTTTTTTGGTTTTTCGAGA
CAGGGTGTCTCTGTGTAGCCCTGGCTGTCCTGGAACTCACTCTGGCCTCGAACTCAGAAATCTGCCTGCCTCTGCCTCC
CAAGTGCTGGGACTAAAGGTGTGCACCACCACTGCCCAGCTTAATTACACCTTTGAAGCTGTTTCCAAATAAGTTATAC
TCTACAGGTAAAGGGGTTTAGGTCCTGAATATCTAATCTAATCTAATCTAACCCAATCTACCACTGATACCCATTTTCT
ATCACTGCCTATGGTGCGTCTCAAAAACGTAAGAAGCTGTTTAAAGTAACATTGTATCTACTCACAATTCAGGACTCAA
CAGTTAGGGCTGGGCTCAGCTGGAAAGTTTTGCTGATTTCTCCTGAGGTCATTAGCATGGTTTTTTGTTTTTTTGTTTG
TTTGGTGTGTGTGCGTGTGTGCATGCGTGTTTGTGTGTGTGTGTGTGTGTGTGTGTGTGTGTGTGTGTGTGTGTGCGTG
TGTCTCCTTCACAGCCTCTCAGGCTCTCCTGGTGGATCAGATGAGGTCTATGCACACGCAGCTAGACAGAGGATAAAAG
CATACGCTTCCATGCCTGCTGACCCTTCCAGCCTGACTGGGCTGCTGTAGGTTTTTATGGTCATGCAAGCAAGGCAAGT
TCAAATTCAAAAGACTCAACTTCTCTGTAGAAGTGGCAGAGGCAGATTACAAAGGGGCCTGCATATGGCAGTAGTGGAA
GTACTGAGCTGTCTTTACAAATGATCTCCGTAGACCTCCAGTTCTGGAAACATAAGGTAAACAGATCCAGGCCTTTCAT
GTTAGAACTCATCCTAGCAGCTGACCAGGCTCCTGTGGGGAAAGCTTGGTGAGTGCGCATGGATGTGCAAAAGAAAGCA
AGGTGCTCTATTGTGAGTGGCAGACAGCATGACTCCCATGCCCTAGGCCCCTGGTGGCCCCAATCAGAGACTAGGATCA
TAATTTAAGTATAAAGCAAAGATGGCCCAACCTCAAACCCTGAAGAAACAGCTCAGATTCATATGCAATGCTCGCTGAG
GCAAGATGTTTTATGACAAGAGCTGGTAGGGAAATTAGACTGGAGTTACTCCTTGGCAGAGTCCCCTGTCCCTCCCTTC
CTGCGGGGTGAGCTCTCTGCCCCTTTCCCCAGTGACTGCTCCACAGAGCTCTGGCAGGCCACACTGCAGTGGGAGGGAA
AGCCCTGGGGGTATATATCGGATGTCATAGGAGGTGAAGTCCTGGCTGCACTTTTCACTGTGTGACTTTGAGCCTGTTT
CCTCATCTGTGAAATGTGGGCTCTCGGAGCTTTCATAAGAACCAAGAATGCTTTGCCAATGCTGCAATCATCTCTGAGG
ACCACACAGCCCCTGGCTTGCTCATGCATGCATGCAATAGTCAACCTTGTCTGCCGAGAGCAACATACTCTTCCTAGGA
CTTTTCTCTGCCTTCTTCATCCTCTGCCAGTCCTCTCAAGCCTGAGCTTTGAATGAGAACGCCATCTTCTTAGTTATCC
TCTCCACTGTGGATCTTAGATTTCTCTCCTGAGGTTAAAACTTTCTTTTTAATTATATTAAAATTCAATTCTGCCGAGC
AGTGATGGCACACGCCTTTAATCCCAGCACTTGGGAGGCAGAGGCAGGCGGATTTCTGAGTTCAAGGTCAGCCTGATCT
ATAGAGTGAGTTCCAGGACAGCCAGGGCTACACAGAGAAACCCTGTCTTGAAAAAAACCAAAAAAATAAAAAATAAAAA
TTTAATTTTAATTCTTATTGGTTGAAATGCCTTTCATTTTTTAAATTAGGTATATATGTGCTGGCAAAGGAGTGTGTGC
ATGAGGGCAGGTCCCACAGATCTAGAGGAACACGCTGGATTCAGAGTCCCTTGTGAGCAGCCACACCAGGTAAACCAGG
AGTGAACTCTGGTGTACCCCTGAGTCATTCTCCTGCCTGACTTTTCTTCTCTTTCTGTCTTTCACTCATGTGTCCTACC
TTCCTTTCTATCTTTTCTTTTCTTTTACTTTTTTTATTGCATTCCCCTAAGATGGGGACTGATCTGATGCCAGGGACTC
CTGCAAGCCATCTACCACTGGACCACATCCCTAGCTCCTTCCCTGAGGTTTCTCAATGTGAATGGCCAAAGAACTGGAG
GTTTCAGAACCAGAAACCTCTAACTGGTTATGTGTCCCACCGTCCTGGGGAAGAACCAGAAAGGAACGGAAGAGGCCAC
TGAATCCTGGGGCTGGGTCAGGCCAGCTCCCAGCTGTTTCTGAAGACTTTTTCTAAAGAAACTTCCCCTTAACATAAAC
TTGCAGTGAAAGGAATTTTCACTAAGACGGTTGGTTTGTTCAACGTTTATTGAGCAATTCAACCTGGCCTACCCTAGAC
ACACAGCAGAGACATCGTGAGTCCTTTCCCCTGAAGAAAGAACAGGCTCAGAGTGTGAAAGGAGCTGTCACTCAGAAAG
GACATGCTGAACTCAGGGGAGGCTGCAAAGGCCTTGTGGAGGGAATGCCTTAAGTCTGTGGGTGAGTCTTTTTGGCAGT
GCTGAAATTCAAAGTGAGAAGTCTCACAAGAGACAGGAAATGTTCTGTTTTTATTTTTATTCATTTTTTTTCTCTTCCC
CCTTCCCTTTCTCCCTTTTGCTCCAGACCCACTCGCTCTGCCCCCGCCCCTCCCCTCGCTCCAGGCCCTTCCCCGCTCA
CTCTGGGCCTTTCTTTTCTTTCTTTCTCTCTCTCTCTCTCTTTAAGATTTATTTATTTATTTTTATGTATATGAGTACA
CTGTAGCTGTACAGATGGTTGAGCCATCACGTGGTTGCTGAGAATTGAATTCAGGACCTCTGTTGGCTTCAGTCCCCCC
TGCTCGCTCTGGTCAACCCGGCTCACTTAGACCCAAAGATTTATTTATTAAAATATCTAAGTACACTGTTAGCTGTCTT
CAGCCACACCAGAAGAGGGCGTCAGATCTCATTACGGATGGTTGTGAGTAACCATGTGGTTGCTGGGATTTGAACTCAG
GACCTCTGGGAGAGCAGTCAGTGCTCTTAACCGCTGAGCCATCTCTCCAGCCCCCTTCATTTATTTCTTAACAAAACCA
AACCAAATAGGGTCTCATGTAGCCCAGATCAGCTCCTGATTCTCCAAGTAGCTAGGGATGACCTTGAATGTCTGATCTT
CTGGCTCAGCCTCCTGTATGCTGGGGTTACAGGTGTGTGCCCTCATAGCCATTTTTGGGGGGATTCTGAGGATTAATCC
CGGGCCTTATGCACAGCTACATTCTCAGCTCAGAGACTAGAATGGTTCAGAAAAGCCTTGTTAGCTTCTGTGAGTGAGC
AGAAAATGGATAGACAGTGCCTTGCTTGGAACTGGAGTGTCCTCATGGACTGGGGCTCAGGCAGAGAAGAATAGGGAGT
CATTTTATTCTTTGGATTTCTTGTTTCAGTTTGGCTTGGGTTTGCTCTTGTTTCCATTTGATTTTCCCCAGATTTTTTT
TTTCCTTCTGGCTTATGAGGGCAGTAGCTCTGTGGAGGTCGTGGGGCAGCACCAACAGTCTAAACAGTTGTCGGTTCCA
TCCTACAGAGCTGCGTGAGATGGATTCTGACCACCTCGCTGTGGATTGGCGCAGCTCAGGAAGCAGTGCAGCTTGACAC
AGAGCTTGGGAAGCACACCCCCATTGATGGTGCTTGCTTTGCAAACATCCCTGATTGCTGCGGCCTCTACAATCCTCTG
AATGACGAACAACTTCATGGCCTCGTCCTTGGGCACACACCGGTCAACATCACTGCACAAGGTTGAGGCCCTTTTTGAC
GTGATCGCTGTTTCTTTTTCCTGTCTGTCTTTCTCTCTCTGTGTCTCTGTCTCTGTCTCTGTCTCTCTCTCTCTCTCTC
TGTCTCTCTCTCTCTCTCGTCACCTTGTAGCCAAGACCTAAAAATTCTTGTGGGTTTTGTTTTCTTTTACTTTCTGTAT
ATGTGAGTTTTATCTGCATGTATGTCTGCATACCACATGTATGCCTAGAGCCTGTGGAGGTCAGAGAGAACTTTGAATC
CCTAGAACTAGAGTTACATACAGATGGTTGTGAGCCTGCCCTGTGCAGAAGCAGAAAGGACTTGTAACTGCTGAGTTAT
CTTTCTGTTTGTTTGTTGTTTGTTTTTTACAGATTTATCCTATTTTTCATCACATGAAAAATAAGATCTTGTTTTCTTA
AGTAATTGCAGAGTGACAGACACCAGAGTAAGGACTTAATGAACCAGGCAGAAGGACCCTTCATCTAAAGCAGCCAAAG
AGTCGAATGATTAGAAGGGAGCATAAGAGAACGTGGTTAGCTCAGTGCAAAGGGATTACCAGGACACATGTACTCAAAG
GCATTGTTAAAGACCAAGGACAACACAGATGCCACCGTGAGGCAGTGTGAACAGGTGAGGCGTGAAATGCACATAGCCA
GAAAGGCGGCAAAGATTCAGCTCCATCTTCCTGGATACTGCCCTGTTTGCCCATCCAACCCACAAGGCACCCGGTTCAG
CTTCTGCAATAAGAGGCAGTATGATGCCCCAAAATGTTTGATACCAGGCTGTGATAGCCCTGAGGACAAGATCTGCCTT
GTTCCTGTGGCAGACCCAGCTCCAGCCTAGACCTGGCATTGGGTAGAGGCTGGCAGTTAAGAGTTGCTAGCAAATAGAT
GGCTTCAGAACTTTCCAGAAAGCTAGTGGCTGATAGAACCAAGGAATTCCTTAGACCAGAGTACAGAATGGCAGGAAAG
CACCAAAGTACACAGCAAGCTCAGACCAGGAGAGTTTGATCTCTGTGTCGAGCTCAGAGATGGAGAAGGACCCAGTAGG
GTATCATGCTATTATACCAGAGCTACAAGGAGGTCCTGGTCCTGGATAGGAGGAACTCATGGGCAAAGGGCTAAGGGCA
AACTAGGAACCTAAGACCTGTGCAACCATAGAAAGGCTCAGTAGAAGGACCCAGATATAGCTCTCTCTTGTGAGACTAT
GCTGGGGCCTAGCAAACACAGAAGTGGATGCTCACAGTCAGCTATTGGATGGATCACAGGGCCTCCAATGGAGGAGCTA
GAGAAAGTACCCAAGGAGCTAAAGGGATCTGCAACCCTATAGGTAGAACAACAATATGAATAACCAGTACCCCGGAGCT
CTTGACTCTAGCTGCATATGTATCAAAAGATGGCCTAGTCGGCCATCACTGGAAAGAGAGGCCCATTGGACTTGCAAAC
TTTATATGCCCCAGTACAGGGGAATGCCAGGGCCAAAAAGTGGGAGTAGGTGGGTAGGGGAGTGGGGGGGGGAAGGGTA
TGGGGGACTTTTGGGATAGCATTGAAAATGTAAATGAGGAAAATACCTAATAAAAATATTTAAAAAAGAAAAAGAAAAA
GAAAGAAAGAAAGGCTCAGTAGGGCCTGGGAAGATAGCTCTGTCAACTTGGGGCTTGGACTTTGATCTCTAAAACTCAT
GTTTAAAAAGTTGTGCATAGAGCCAAGCATGGTGATACACGCTTTTAATCCCAACACTCAGGATGCAAAGGCAAGCTGA
TCTCTATGAATTTGAGACACAACTAGACTACAAAGCAAGTTCCAGATTAGCCAGAGCTATAGAGCGAGGGAGACCCTGT
CTCAAAAAGAGACGGGGGTGAGGGGAGAGGAGAAGGAGGAGGAAGGGGAATGGAAGAAGAGGAAGAAGAGGAAGGAGAA
GGAAGAGGAGGAAGAAGAGGAGGAGAGAGCTATGTGTGGAATGTACGTGTAACCCTAGGGTTGGAGAAGCTGAGAAAGG
TGGGTTCTTGGGGGCTCACATGGCAAGCTTTAGGCCAGAGAGAAACTAGTCTCAAAAAAAAAAAAAAAAGAAAGAAAGA
AAGAAAGAAAAGATGGTGTTTGAAGAAGCTGATTTTTGGCTTCCACATATGTTTGCATGTACATTAGCATCTGCCCTCA
CTTGTGTTCCTACACCACACACACACACACACACACACACACACAGACACACACACACACACACACACACACACACACA
CACATACACAGCAAAAGATCTGAGGTCTGGTCCAGCTATCTCTGGAATGTTCATTATGGTCAGGCCTTGTATCCAGGCT
CAGTAAAAGTCCAAGAACTGAGTGGCCAAGATCCATACTTGGGAACCTGAGTCAGCCCTGGCAGAGTTGGTAGATACTG
CAATCGGGTGGGCAGGAGCTATAGGCAGGGTCCCTGCGGCTTGTCAGAATGTTGCCGCCTGGGCCCTTTCAGCTGGATG
CTGGGTTGGTAGGGGAAGAGTACTAGTAGGAGCAGAGCTGTAGGCAGGGTAGAGCTTTGGGTCTGACAAGGTCAGAGCT
ACTCCTGTTCTGGAAAAAGAAAGTCAGGGTAAACATCTGGCCCGTAAGTACCATGAACAAGCACTCAAGGGTACAGGAA
AAGGGTCAGAAGTGTGGCCTGAAAGGAAGTCGAAGCCATCAAGGAAAGCCAAGAGTGTTGAAGCAGGGTTGAATGCTAC
CTTGGCTGTATGTGTGACACTAGAGAGGAGATGTAGGTGGGAGTGCAGTCAGGCCCACCATCCATGATTCCAAAGCCCA
CCACACCTTGCACCATGCTCACTCTTTAACACCTTTGACTCAAACTCTCAACAGCAACCTCCGTGAAGGCCGAGGGGCA
TCCGAGAACAGGAGCAGGCAAGAAGCCTGTGCCTCTTCATCCCTAGACACATCCCTTCTTGTAGACTGACTACTGGGCA
GTAAACCGTGAGGATAATGAAACCCAGAATCCCAGGATGGAGGAGGATGCTGCGTGGCTAGCAAGCTTCAACCCTTTTC
TAGAATGGAATCCTCTTTAGTAATATAGTGGGTTCTCAAGCAACTAGGTCTGGCCTGTAGGCCTGCCCCACCCTGCCTG
ATCCTCCCAAGCCCTGGCTTCCCGTTGGGCTGGTCCTACCAAGGTAGAAGGCTCCAGGGCCATATTTTGGGCCCTTCCC
TCCCCTATACCTGCGTCAATGACTCTTTCTGCCTTCCACCCTAGCTTTAAGGAGGGTGCTCATCGGAGGCTTGGCTCCT
CATCAACAACCCAAGGCTCTCCAGCCTCATCAACTGTCAGGCAGATTGGGCTTGGCTGTGCTTCTGATCCAAGTTCTCC
ACCACTGCCTACCTCAGCACCCTCCAGTGATGTGCAAGTTCCCAAGACCCACCTTAATGCCCCTCTGCCCAGCTCCAGT
TGGTGGAGACCAAGCACATGGGCAGGGCAGGAGTGTGAGACTTCCTAGCCTTTCCCCCTTGAGCCGACCCTTCAACTAC
CCCCTCACTTCATTCCTATGGAAAGACCAAGCCATGGTTTATAATACTTCCTCTGAACTCTTAGGAGGAAGATTTTTAT
AAAGCAGGGACTTGAAGACTGGTAACACCATGTTCTGAAGGTTGCTGTAGTTTGCAATTAGTTCTTCACCAAAGCAAGA
AAGAATAACAACATCAGCCACAGGCCTGGGGGGTTGGGAGGGGAGCAGGGAAGCTTGAAAGCCAATTTTTTTTATATTT
AAAATGTTTTATTTTAAAATATTTTTATTATTTTTAATGATGTGTGGAAGTGTGTCTGCATGTAGTGTCATGTGAATCC
AGGTGCCTGCAGAAGCCAGAGCCATTGAACCCCCTGGATTACAGCTACAAGGGCTGTGAGCCACCAGATGTGGGTACTG
GGAACTGACCTCAGGTCTTCTGGAAGAAAACAATTGCTCTTAACCTCTGAGTCATCTTTCTAGCCCCATAAAATGTTTT
ACATTTTTGTGGATGTCTCTCTTGCATGTATGTCTGTGCCCAATTTGCATGCCCCATCTCTGTGTTGGACATAAGAGAG
CACTGGATTCCCTGGAATTGGAGCTACTGATGGTTGTGAGCTGCCAAATGAGTGCTGAGAATGGAACCTGGATCCTCTA
TAAGACTGACAGGTGCTCTTAACTGCTGAGCCATCTTCCCAAGGCCAAGCATCATTGTCAGGAGCTACTGGACTGCCAT
GAAGAGTAGACTGTAGCAGCTCTTGGCAGTTTTCCCTGGGGCTGTAGGCAGATGATAAAACCCACAGTATAGGAGTAGA
TGTGCCCGACAGAGGAAGCAGAGACTCTATAGGAAACACAGAGCTGAGGCTGGGTACCTGGGAGCAGCCAGAAGATCTC
ACAGTGCAAGAACATGCAGTAGATGCAGATGAGTTGGCTGCATAAGGAAGGAGAGGGAGGACAGGAAATGGACCAGGCT
GATCAAGATACCAGAAGAATTTCTATACAGCTAGAGTGAGGAGAGCGTGGGTAGGACAGGCAACCCTCAGGGAACGCCC
AAGGTTTGGAAGAAGACTCTTTGGCCAAGTGGTCATTTGATAAAGGGGGGTATGGCTTACCATTGAGGATCCTGGCCAT
GTCTCAAGAGAATACCCTCGAATACTCGCTCACTCCTGCTTTGACCCCATGAGCTTTAGATATGACTGAATCTGACACA
CACACACACACACACACACACACAAGGGGGGAGTATTGGATGTGTACAAGTGGTTTGCTGACATTCATGTCTATGTACC
ATGTGTCTAAAGTGCCAAGGGGCCCAGAAGAGGGCATCAGGTGCCCTAGGACTGGAGTTACAGTCAGTTAGGAGTCACC
ATTTGTGTGCTGGGGATCAAACCCAGGTCTTCCATAAGAGCAGCCAGCACTCTTAACCCCTGAGCCAACTCTCCAACCC
CACATGGCTGAACCTTTAAAGCCACTAGAGTGAGTTCTATCAGCTGTTCATCTGTCCTCCTGGGACAGAAGTGGCAATC
CTGTATACTTCACTTTCTTAACCATGTAAGGAACTTATTTTGCAAGTAAGTGTCTGGAGCTCAAACTGACCCAAGAATT
TGACTGAAGTCACACAACACATCAGACACAGAGGAGGTTCAAGCCTGTCTAATATCCAAACTCTGGGTAATGTAAATAG
TTGATATATGCTTGTCACACATATATTCAACAGATTCCCTGGGCATGTAAACTTGGGAAATGCTGGTTTCAGTGAACTT
GAGCAGACTCTTTTCACAGCATGGAGAAAAACTTTAGGCCATGGACTCTTGTCCCAGTTGGAGTTGCTGTCCCTTAGAA
GCAGGACCTCCTGTCCTGTAAGGTCAGAGGTGGGGCACTGGAGGCCTCTTACTTCAAGCTGTTTATATTCTCACTTTAC
ATCTATGGCTCTCCCCACAACACATACACATCTTATGTGTGAGGAGAGGGGACTGGTTTTTAATTAGACTTTGAAGAAT
CTTGGGTGAAGGATAGTGGGGAAGCCAAACACCTCTGACCCTTAGTTGTCACAAGAGATACCTTCATTCTACGTACAGG
GCCTAAGGCCTTGATCATCTATGGTGACATGTCTTTGGGACACTGCCAAACAACAAAGCCATTTCATGTATTAAAGTTT
GTTTGGAAGGGACTTGTGGATGTGGTATCATTTTAATAAAAAGCTGAGAGATTAAGAAGCAGAGTTGAGGTGGAGAGAA
AAGAGCCACATTTAGGGGAAAGAGCATACTCACATAGCCCAGTCCTGGGAGAAGCCAGGAATGGAGCTGTTCTTGATGC
AGCAAAGAACTGGATTTGGATCTGCAGGCATGAGAACTATGGGAGATGCTTGAGGCAGAGGTCATTTGATGCAATCTAG
GAGTAGGGACTGGGATATGAGTCAGTTGGTAGAGTGCCTGCCCTTTGTGCACAAAGACCTGGCTTGGGTCCCCAGTATC
GCAGACACAGAGACTGGAGGTTCGTGCCTATAATCCCAGCTCCTGAGGGGTAGAAGAAAAAGGATCAGAAGTTCAAAAC
GTTCTCTGCTACATAGCACAGTCGAGGCCAGCTTGGAGGCCACACACACACACACACACACACACACACACACACACAC
ACAAACTCTTAAGGCACCCCCCCACCCCAGCTTCTCTCTCATTTCTTAATGGAATGCCAGCTTGCACACAGCTATTCCA
CATCTTGCCTTTTTTCTCCCCTCCTCCCTCCTCCCCTCCTCCAAAGCCTCTTTGGCCCAGCGTCTTTGTCAGCAGTGTT
CCCTTAGGTTCTCGCTGTATGAAGCATCTTTCTGTAACTTCTGCTGGAGAATAGCTTCTGTGGACACCACGGAATGTCT
GGTTATTGAGAAAGTGCGGAGAGATGACCTTCCCCCCTGAAGAGTGCCATTGGCACACTTCCCTCGACCCTTTCTGCCT
CCTGAGATCAGCCTTCCTGAAGTCCATCCCTTCCGGGGAATGGGGCATGCCAGTAGGGTCCCCAGGGACATTCTCCCCA
GCTATGCTGGACACTTCCCCACCCTCTAGAGTAGATGCCAGAAGAAGAAGGGCAAGTGGAAATGGGGGTGGGAGTGGAG
GTAGCGACAGAGTGGAGACAGAGAAAAGGGTCTGACTGAGGCCTGTGACCCCTGCTGCCCCCAACCCATGCTTGTCATG
AATAAGGCCAGTGGATGGAGAAACACTAGTTTCTTTCTGGCTTCAAGGCTCTGGTTGGATTACTCCTTTGATGTGTTTG
TCCTTTGGCCTCAAAGGGACGGCTTCGACTTGGTAGCTCATGATAGCCTTAAGTACAGCAATCCAAGGTCACCAGGCCC
TCTTTCTAACACAAGTCCCCTTCTCCCTTGGCTTTCAGGAAGGACCAAGGCCCCAGAGCCTCGTTAATCTGTTGTCAGA
GTCCCTGATGGCATGCTAGGTATTTTTACAATTGTTCTAAAATCCCATTATCTCCACCGTCCGGGTTGCACAGGGGCCG
CCATTGGCTGAATACGTGACCGACCACCAATGATTCCCTCCAGGGAACGCCAGGCAGAGCTCCCTGGGCTGCTGGCCAT
CATCCAAACATGCTCCTCTCCGTGGAAAAGAAGTGATGTCATACACGGAAGGGGTGGTCTCCACGCACAGGATACCTGT
AAACACCGAAGCATGTTTCAAAGAAAAACAAAAGTCAAATTACAGAGCTGCAGAGCTGGGAGGGGCTCGGTGTCCACTT
TACTCGTTCTCTCAAGGCAGCCCAGGCTACACCGGTCACTTCCCACTCACCTGTGTGTGTTATTATCTGCTCAATTCAT
CTAGAAAATATTTATTGACCAACAAGTATTCTAGATGCTTGGGTGACATCCAGAAACGGAGATGAGCCTCTCCCCTCCC
AAGGGCTTGTGTTCCGTGGAAAGATCATTGGTAAAAGGAGAAGGCAGAAAATGTTAGAAGGTGATACGCGCAAGACAGA
TGAGCCAGGTGAAGTGAAGCTGGGGAGAGCCAGCAGAGGACACCTCTATGGAACATTCCAGAAGAAGCAGTTAGAGAGC
AGCCGGTCAGGAACAGGAGAGAAGTAGAGCAGAGAGAGGAAGCCGGCGGGGGAAGCCTGAGCTCACTTGTCCCAGCTCA
CAGTGAGGACTTAGGGTGTTAGGAGAGCCAAACAGGGAGCCAGTGAGGGAAGGGGAGTGACACAGTTGTTCTTTGTTCC
CAGGAGAGCACCTAGAAGGTGAGGTCTACCTGCAACTCCATGTCTACACAAGGTCTGACAAGAGTGCAAACGTTCTGGA
GCCTGGAGATGGCTCAGTGGGCAAGAGAGCGAGCTGAGCAAGCATAGAGATTCACGTTCGAACCCCCAGTACCCATGTA
GAATGCAGGTATCGCCTCTTGCATATCTGTAACTTCAGTGCTGTGGAGAGCAAAGACAGGACAATTCCAAAGGCTTGCT
AACCATCAGTCTATCTCCAGGTTCAGCGAGAGAGCCTGTCTCAAGGGAGTAGGGCAAAGGGTGCAAACACATGAGCATG
AACACGTACGCACATACATACATACATACATACATATACACATACACACATACACACATACACTCCACACATAGATACA
CCATATACTCACAAAAATACACATGCATGCACACACACCACACAAATATACTCCACACATACATACACAAAAATATACC
ACATTCTCGCAGACATAAACATTCATACATATACCACACAAATACACTACCCATACATACCCACACAAATACACTATAT
ATACATACAGAAATACATCACATATATACCCACACAAATACACAATATGTATACACACAGAAACACACCACATACTCAT
ACAGCCAAACATAAACATACACCATACAAATACACTACAAACACCTACCCACACAAATGCACCACACACTTAAACACAT
AAACCTGTACATGTATACACACTACACTAATGCACACATAAATGCATCGCACACACAACACACATGCTCACACACACAT
ACCACATGCACACACACACAGCACAACACCCATACACATTCAACACACACCACACACCCTCTCACACACACTACACACA
CATCTACACATTCAGCACATGTGCACACCATACATACGTGCATACCACCACAATACCCACCACGCACATACACACACAT
ACACACACCACACTCTTACACACATACACACACCATGTACACTCCCTTCATACGTGTACAGAATGCAAAACTTCCGCTG
GAATGAATGAAGAAATGACCCTCATACTTTCCCCCCCCCAAAAAAAAAAATAGGACAGAAGGGCAACTGGACAAACGTG
CCTGCAATCTGGGAACCGTATCTAGAGGTTTGTGCTTCAAATGCCTGGGGGGCTTCTCACGACCCTTGTACACAGATCC
TTACCCACAACCGTGATTCCTAAAGGTTAGGACGGTCCCGGACGGGATAGAACATGAGCGTACCACTTTAAAAAAGGAT
TTCTTTGTTTTCCTTCTGTCTGTATGAGTGTTGTGCCTGAATGCATGTATGTGCACGGTGGGTGTGCATGCCATACAGT
GCCTGAGGGGGCGAGCAGAAAGCACTGGATCCCCTGAAACTGAAACTACTGATCATTGTTAGCCACTATGTAGGTGCTT
GGAACTGAACCCTGGTCCTCTAGAAGACTAACAAGTGTTCTGATCTGCGGAGCCATCTCTCCAGCCCCGAGAGTATGTG
CACTCGCTCCGTACTGGGGAGGGAGCACCCCACGGAGACACTCTCAAACACCTTTAAATAAATGATTATTATAAATGAT
TGTTAGCTAAATCCACAGATGTATGCATGCCTGAGTGAAACGTTTGGTTGCTCAACGCCACTTCAACCGAGATGGTTGG
AACTATTGTTTTTTTTGCTGTGACTCTTACTATGATTATCATGATTGATTGTTTGTTTTTTCTAGACAGGGTTTTTGTT
TTATGTGTGTATGTAACAGTCTTGGCTGTCCTGAAACTTGTTTAGTAACCAGGCTGGCCTTGAACCCCCCAGAGATTCA
TTTGCCTCTGCATCTTGAATGCTGGGCAAGCACTCTACCAGCTGAGCTACATCCCTAGCCTCAAACACTTGGAATTATT
ATAAACAAACAACATTCCAGAGAACAGGAAACCCCATGCTGTTGCTTCCTGTCCTGCATCTACCCACGGCCCTAACAGG
ACATCTCCTCTGGTTTCTTATGTTACAGTGGTTTTTACCTGCTCTGTCCTGTCCAAGCTAGAGTGTAGGGATTTTAGAG
TGTCTAGAAGCAGACTCCTGCCTTGCACACCCCTGTGTATTCCTATGGAGCTCTGAACCAGTAGGGTTTCTCTCTAAAT
TAGAGGGTTGCTGAGGAATGAGGGTCTACTAGATAGCCCAATGTCCCCAGAGAAAAATATAACAGAGCCCGGGGTATAG
AAGCATGGGGGTGTGACAGTGGTAGAGGGGACAGGCAGGCAGGCTTCATCCCATTTCTCTCAGGAGGATGTGTAGACAG
GCTCACATGTCAGAGCTGCAGGGGCCGAGACCAAATGCTGCAGGGAATGTTGGAGCCATAAGCACAAAAACCAAGAACC
AGAAGTCCCTCCCGTCCGCAATGTCCCTTCGCTTAGCCTGATGGCATCCAGAGAGAGCCTGGAGCGCCACTCTGGGCTG
CTGAGTCTCACGATTCTTGGCCTCTGGCGTGCAGGTGCTGGCTGCCTGAGATTTTTCTGCCTGTAAACAGTCAAGGGCC
CCTGATTATCTTACATAGTTGGAATAGGCACTCTCATTTTTTTCCCCTCCTCTTCCCAGAATTCAATAAAGGCCAGAGA
TTCCACAAGCCATCTGACTCAACTTGATTTCCTCCTTCCTCCTGTTTCTCCACCCTATGACCCTGAAAAGGCCCAGAAC
CCAAGGCTCTGCAGCCAGGCCCTAGCCTGTCATTCCAGCTGCTGGTCAGGAGTCTTTAATCTTATTGGTCACCTGGTAC
ACTCGACAGAGTGGACAGCTCTATACTGCAGGAGAATGGTGGCAGGAGGTGGGGGATGGGGAGGATAGTCAACAGCCTA
GTACAGATCAGGCATCCTGAGTTCAAATCCTGGCTCCCCAATTAACAATATGGGGAATGAGTTATCCTGTGTGAGCCAG
TTTCCTCCCGTGTAAGTGGTGTGTGTTCATGGTTTCTTTTCCTTAAGGAGGCTGCAAGGATTGAATGGACAGTGCACAG
AGTTCCTAACAACGGCTCTAGTGATTATATTTCCATCAGATAAAGAGACAGGAGGCTCAGGTCTGCGGGATTGACTGGG
GTTCAAGATGGAGGGAACACTAGGATTGGCATTTTACAAAGAGAGCAAAAGGGAAGGGGTCCAGGAAAAGTTTGGAATG
TCTAACAGGAGCCCTTCAAAAAGTTGCAGAGCTGGGCTTGATGGTGCGTGCCTTTGATCCCAGCACTTGGGAGGCAGAG
GCAGGCAGATATCTGGGAGACCTACATAGGCCTATCTACACAGGGAGTTCTAGGCCAGCCAGAGCTGCAAAATGAGAAA
CAAACAAGCAAACATCACAGATAGAAACAGCTGCATAACAGAGGACTCTGAGCCCAGTCAGCAAGCTGGGTGAGAAGCC
CAGGGTGTCCAGTCTAAACTTCGAAGGTGACAGCACTTAAGATGAACAGTGTCCATCTTAGGAGGCCATGGAAGCCTTG
CCAGGAAGGACCTCACCTCAGGTCTGTCTCTTCTCTCTCTCTGCTTTCTCTGTGGTGTTTGACTTCAGAGATTGACCCT
GGACCCCCTTTTTTCTCTCTCTGTCTCTGGCTCTCTGTGTCTCTGTCTGTCTCTCTTTCTCTCTCTGTCTCTCAGTCCC
TCTCTTTGTCTCTGTCCCTCTGTCCCTCTCTCTCTGTCTCTCTCTCTGTGTCTCTCTCTAACACCCCATCTTGCCCTTA
CTCCCCTCCACATTCACAGTTCTACTTGAATTCTGAAGACCATGTGAGTTCTGAGTCCCCCCTGCCTCTATCATGCTGG
TCTCTCTGAACACTCTGCTTCTCCCCAGCTGCAAATGACAACTTCTCAGGTGTTTGGGTTTTTGGAAGAAAAAGGAGTC
CAGACACAAATGTTCATGTCTGTGGGCCACTTAAGGCAGATGGAGACACCCCCTGAACTATTGCTTTATGGCATGTTTC
TGTGAGCTCCTCTCTCTATGCACATCTGGTCCACCATCATATAGCTACTACTACTATCCTGGAACTCACTCTGTAGACC
AGGCTGGCCTCAAACTCAGAGATCCACCTGCCTCTGTCTCCTAAGTGCTGGGATTTAAGGTGTGCACCAGCACCGCCTG
GTGAAAATACTACATCTTTGGAAAAGGGTATGCCTGCTTGGCATCAAGATAGTATCTTGGCTCTCCGGGTTTTTTGTGA
CTCAGATTTGGGGAACCCTTTCATCTGCCTCTGTGCCTGTGATAACAGAGCTCTGTGGCTGAGTCAGGATGCTGCTGCT
CTAGCCTACAAACCCTCAGCCCAGTAGAACCGAGTGATTATGACCAGTCACGACACATCCTATCCTGGAAACTGCTGAG
AGAATCCATTTCCATCGTTACCATTGTAAGCTAAACAAAAAAAGAAAAAAAGAAAAAGAAAACCCGATGTGAGGCAGGA
TCTATGTTCCTTAGCTCAGTTTGGCTTCAACACGTGCATGTTTCCAAATGCCATAGACCATAAGATACACAATTTGCAT
TTACCAATTAAAAAATACTTTAAAGGTGAAAAGCGGAGCTGAGGTCAGGCTAAAGGAGAAGGGCTGGGAGAAGGTCAGC
CAAGCTACCTCCAAACCACACCCCGCAAGTCAGCTACAAAGGACTTTGCAACTTTGCTAGAGGCTGGAGGACATGCCGT
TGCTATACTCAGCAATCTAGGGCAACGAGAGCCCTGCCCTGACCGTATTTTAGGGTCCAGCTATCTGCATTTCCGCCCC
CCCCCCCCAAGCACCCAAGCTCTAGCCATGCTGGGATGTAAGCAGAAGAGTGCACCTCTCCTCTGGTAGACTCAACTGG
GTCTCAGTTGTGGACCCTTTGTAGGGTAACTGAAGGCCTCTCTTATTAGTGAGAGGTATGTAGGGCCAGGAAGGAAGAG
AGGAAGCTGAGGAAGAAAAGGCACTGGCTGACTGACCGCTGTGAGAATCTTGTACAGCAGGAATTACTGAACCCATTGC
ATGGATGAGATTAGGGTATCTGTCAAGGTCATTTCTACACAAAGAAGAGCTAAGCCACAGACTCCCATCTGACTGACTT
GAAAGCAGAGGCCTTCTTTCAAAAGCTTAAGATCTCGAATCACACCATGGCAATACAGCAACTCTATTTACTATGCATA
CATGTGTTTGTGTAATTAAGATTGGGTCTCATGGAACCACAGCTGGCTTTGATTTCACTTAGTCTAACAAGAACTGTGA
GCATCCCTCTGTATCTCTCAGGTGTTGGGATGACAGGTATGTACTGCATACCCACAGTGCTGGGAACCAAACCCAAGGC
CTCATGTACCCTGGCAAATATTCTCCCAACTGGCCTACATCCCTACCCCATTACCTAGTGTTTTTTGTTAGTTTGGCTT
TTGAGACAGCATCTTACTCTGTAGCTCTGACTGATCTGAGACTTACTCTGTATCCCCAAGGGCATTGAACTCACAGCAA
TTCTGTTTCAACCTCAACTGAGATAACAGATGTGGGCCACTAGACACTGGTTAATTTTTATTAGGGGAAGGGGCATTCC
TGGGGTTGGAACCTTGTGTATTCTAGAGAAGTTATCTACCTTTGAACTACACTTCCAGGGTAACCTTTTTAAAATTCTC
CTTCTAATTCATTTCTTGTTCTGGTGCCAAAAACTATCATGTATTTTTATGGTTGCTCATTTATCACTGTATTTTAATG
TTTATAATGCTGTTAGTCTTTATAATCATCACTTTTGCTTGAACGGAGTGGTGCATGCCTATAATTCCAGCATTCCAGA
CATAGAGGCAGGAGGATTGGCCACAAATCCTCAGTGAGCATGTCCTACAAAGAATTCCAGTCCCATCTGGGACTGTAGT
GTGCGTCTCTGTCTCAATTAAAAATAATAATAAAACCACTTTTACACCCCAGTCCACCCTGCAGACGACATATGAGTCA
GTCAGCCATCAATGAGCATTCAATGCCTCAGCATGGTAAATTGTCTCCACCCAACCCTGGTCTGGGAAGCAGGGCTCTC
ACCTGCCCTGTCTGGAAGGTTATGTAGGTCACTGGAACCTGACTCGACCTGCTCTATCGTAACAACGCGTACTAAGAGT
GTCGGGAAGGTCCCCAAATTCTCTGCATCTCCCTGTGCTTCCCTCTTATAGGCCCGGCCTGGTCTGTTCCCATGCACAC
AGGCCACAAAACTGGAAGGCACCAAGGTAGTATCTTGCTGGTGGTGACATGTCCCCGCACACAAGGACCAATGAGAACG
ACACCAAGTCAGCCATGCTCCCAGCACTGGGATGGGAGCAAACTCCCTGCCAAGAGCCCTGAGTAGCTAAAGCTTGATG
CCCATTCTCTTGAGAAAGCCCCTTCTAGAAAGTTCCTCTTTCTGGAAGCAGAAGCATTTTAGGCAAGGCGAATAGGCCA
GCGCAAAACGCCCAAGGTGATGGGCCGAAGATCACATTTTCAGGATGCCTCTCTGCTTTATGTGTTTGCAAAGCAGATA
TTTGGGGAGGTTGGGCTCCAAATTGCTTCATGGTTGCCAGCCTCGTGGGAAATGGCTCACATTCTTAGCATAGTTAAAG
AGCTGTGAAGCAACAGAGAAGATTGCTCTGAGGCCTCTGGGGAGCTAAGGAAGATGAGCCACGGCGTAGGCATGCCTCC
CTGGACGGGTTCTGAGATGGGATGCACCAGTTATTTTTTTTCCAAGAAGGCAGGGTTGTAGTGAGTTTCTGAAGGGTCC
ATTTGTGAGAGTCACTTCAGGGCTTATGCAAGCCCAGGCCTGATTGCTTTCCAAGACCACTTAAGCAGCCCCTGACCAG
ATAAGCACTCTTCTAAACAGGCAGAAGTTAGCTTCACAGCATTCATGAGAAAAGCTTACGCTATACCCACCGGGTTGGC
AATTAGCATTTCTCCTTTTTCTTCTCCTCTTCCCTTTCTTCTTTTCATCCTCTCTTTTGTAGCCCAGGTTGGCCTCGAA
CTCTCTATAAAGAGAACCTTCTTATATTCCAGCCTCCATCTGCCATGTGCTAGGGACTGAGATTTCTAGTTGACTGAGA
TTTCTGGGGTTTTGTTTTTGTCCCCTCACCCCAAGTTCCTCCCTCCCCTCAGTATACTGCCGTTGAAATTTTTCTTTTA
ATTGAGAAGGCATATCATTTCAAAATCAACATCAGTAACATTTTTATTTATCCTTTGAGAGCTTTATACAGGTATATAG
TTTATCTCAATTATATCCACCTCTCCAACTCCTCCAACACATCTTTCTCAACTTTATATTCTTTATTTCCTTCTCCTCC
TCCTCCTCTTCCTCCTCATCTTCCTTCCTCCTCCTCCTCCTCCTCCTCCTCCTCCTCCTCCTCCTCCTCCTCCTCCTCC
TCCTCCTCCTCCTCCTCCTCCTCCTCCTCCTCCTCCTCCTCCTCTTCTTCTTCTTCTTCTTCTTCTTCTTCTTCTTCTT
CTTCTTCTTCTTCTTCTTCTTCTTCTTCTTCTTCTTCTTCTTCTTCTTCTGTCACTGAGTCCGATTAGGGTTGCCCCTG
TGCTGATGAGTGTGAAGTTACTGTTTACTGGGTCATAGGCAGTCTACAACAGCATTTTAAAAAATAAGTAACCAAGTAA
AAAGGCCCTCAGGTCAAAACTGTTTATTGCCTGTCTTGATAGTAAACCCCAGTGTCTTAGTCAGGGTTTCTATTCCTGC
ACAAACATCATGACCAAGAAGCAAGTTGGGGAGGAAAGGGTTTATTCGGCTTACACTTCCATACTGCTGTTCATCACCA
AGGAAGCCAGGACTGGAACTCAAGCAGGTCAGGAAACAGGAGCTGATGCAGAGGCCATGGAGGGATGTTCTTTACTGGC
TTGCCTCCCCTGGCTTGCTCAGCCTGCTCTCTTATAAAACCCAAGACTACCAGCCAGGGATGGTCTCACCCACAAGGGG
CCTTTCCCCCTTGATCACTAATTGAGAAAATGCCTTACAGTTGGATCTCATGGAGGCATTTCCTCAACAGAAGCTCCTT
TCTCTGTGATAACTCCAGCTGTGTCAAGTTGACACAAAATTAGCCAGTACAATTGACCCCTTGTCAACTTGACACACAA
AAACATCACTAGTAAGCCTCAACCCTTACAATCTTATTCATCCCCAAGATCTAAATAACTTTAAAAGTCCCACAGCCTT
TACATATTCTTAAAATTTCAATCTCTTTAAAATATCCATCTCTTTTAAAATCCAAAGTCTTTTTACAATTAAAAGTCTC
TTAACTGTGGGCTCCACTAAAACAGTTTCTTCCTTCAAGAGGGAAAATATCAGGGCACAGTCACAATCAAAAGCAAAAG
TCAATCTCCAACCGTCCAATGTCTGGGATCCAACTCACGATCTTCTGGGCTCCTCCAAGGGCTTGGGTAACTTCTCCAG
CCAGGCCCTTTGTAGCACACGCATCGTCCTCTAGGCTCCAGATGCCTGTACCCCACTGCTGCTGCTGCTCTTGGTGGTC
ATCTCATGGTACTGGCATCTCCAAAACACTGCATGACCCCTTCAGTCCTGGGCCTTCAATTGCAACTGAGGCTGCACCT
TCACCAATGGCCTTCCATGGCCTCTCACAGTACCGAACCTCAGCTGCTTTGCGTGACCCCTTCATGCCTTCAAAACCAG
TACCACCTGGGTGACCCTTACATATTACCAAGTCCCACTGCAGCAGGAGTACATCCTTGGCCATCTCTGGAACACTGCC
TCTTTGTGCTTTCAGAAAACACTTCCCAGAAGATGTCACCTCAATGATGCTGGTCTCTTCTTAATCACCGCTAATTTCT
TAGCTCCAGCTAACCAGCATCAATAGTCCCAGTAATGCAAAGTTTTTGCTTTGGTAGTTCTGGTATCTTGTTAATCACA
GCTGATTCTTCAGCCCCAGCTAACCAGAACTACAGAATCTTCACAAACAAAACAGCAATGGCCCTGAAAAGAGTCTTTA
ATTTTTCCTCTGAAATTTCACAAACCAGAGCTCCATCTTCTGCAGTGTTCTCAACATTATCTTCCAAGCTCCTACATGA
CATCCGACAGAGCTCTTAACAACGGATGGATCTTCAAGCCCAAAGTTCCAAAGTCCTTCCACAGTCCTCCCCAAAACAT
GGTCAGATTGTCACAGGAATACCCCACTCTGCTGGTACCAATTTGTCTTAGTCAGGGTTTCTATTCCTGCACAAACATC
ATGACCAAGAAGCAAGTTGGGGAGGAAAGGGTTTATTCGGCTTACACTTCCATACTGCTGTTCATCACCAAGGAAGCCA
GGACTGGAACTCAAGCAGGTCAGGAAACAGGAGCTGATGCAGAGGCCATGGAGGGATGTTCTTTACTGGCTTGCCTCCC
CTGGCTTGCTCAGCCTGCTCTCTTATAAAACCCAAGACTACCAGCCAGGGATGGTCTCACCCACAAGGGGCCTTTCCCC
CTTGATCACTAATTGAGAAAATGCCTTACAGTTGGATCTCATGGAGGCATTTCCTCAACAGAAGCTCCTTTCTCTGTGA
TAACTCCAGCTGTGTCAAGTTGACACAAAATTAGCCAGTACACCCAGATATGCTATCTTTCTCATCCAAAAATTCTAAA
TCCTATATCCTCTTTCCAGCAGATGATTCTATGGCCAGCCACCCAGCTGGAAAAGAATATAGGGTCAAACCTGTCACTG
GTGTGTGTGTTCCCTGCTGGCTACAGTGGCTGGTGGCCAAAAACCACACCTATTGAATTCCCTGAACTGAGAAAGCAGC
ATTCCTGAGACCACTCAGTAGTCCAGGCAGCCTCAGACAGCCATGCCAGGAAATGCAGAGGGAGAAGAATTCCTTCCTG
GTACTGCCTCTGGGGCCAGCACACAGGTCCTCCATCCTGCTGCTGGGGTGGCTGAGCACCTAGGGAAGTTAACACACAT
GCCACAGAAACCTTGCCCACTGAGGCTCTGAAGGGAGTAGTACAGCGAAGCAGCTCGCTGTTCCGTGTCTGTCTGAATG
CGAACCAGGCAGAAATGCTCCCGGGGACTTGAGGCCAGGTGTGTGACTGGGATAAGCAAGGTCCTGAGACTTGGGAGGA
GGAATGAGAGGCATGTCTCAGAAGTGTGGAAGCCCAGCAGGTGTGAGTGCTAAAGGGTTGAAGGGAGACAGGGCCTCCC
TAGAGTTGGAACCTCCTGTTAAGCAGAGGTTCTCAGCCCTACCACCATCTAGAAAGCTCTTAAGGACGGCAGTATACTT
TATGCATCATAACCAATTTAACCCAATTCCTCCAAGGTGGCACCAGGATTTTTTTAAACATCCCTATAATTACAATATT
CATCAAGGCAGGAGAATCAGTGCTTTGAAGATATTAAATGTAGGAACCTCTTGAGTGATTCACCCGCCATCTTTTCTCT
GCGGGACTGATTCTCAAATCTCTGCAGTTGCCTACTGTCCCCATTGCAACCTATTTCCCTGCTGACCACCCAGCTGCAG
TGAGCCTAGCCATCTGAGGTTTACAGTCACCTAGGACCCAGGCTTATGACCACGTGTAGCTACCAGCAGGCAGGCGTTT
ATCTGTCCAGTGAAGCCGACTATACCCATTGGGGCAGGTCCTATGGTGCCACTTATGATTTCTTGACACCAGCCGACCT
ACCTCCTACATCCATGTAAGCCACATCGCATGCTCTGTTATTTCCTGGGTGATTTGCATACTAAGGCAATCTGACATTT
CAAGCAACCTTGATGGGAGAGTTGACAAAGTGTCCAATACAAATGAACAGCCTCAGCCAGGCGGTGGTGGCACACGCCT
TTAATCCCAGCACTTGGGAGGCAGAGGTAGGCGGATTTCTGAGTTTGAGGCCAGCCAGCTTGGTCTACAGAGTGAGTTC
CAGGACAGCCAGGGTTATACAGAGAAACCCTGTCTCAAAAAGCCAAACCAAACCAAAAAACAGCAAATGAACAGCCTCA
CTGTCCAGGAATAACCCTGGTCATGGCAACGGGTGTGTGCAGTATAGGGGAATATAGGAAGAATGGCCAAGCCATGACA
CTTGGACTTTCTCCAGGGAACACAGGCACAACTCACATAACTAAACCCTGGATTAGTTCTCTTTTTGCACTGATCATCT
TCAAAGACAGGGTGGGCAGTTCCATGCAGCAGTGTGGGCAGTTCCATGCGGCAGTGTGGGTGATAGAGGCTGTTTTCTG
TTAAAGACAACGTCCGATATTTGCTGCAGTCACTTGAAGAAACTTCAGTGTGGTATTCCAAACTCATGCATTTATATCT
GCTCTCTTTATTTCAAAAGGAGCACCCAGGAAACTGGTAAATGGACTGAGTGTTTAAGAGCAGGGGGAAATGGGGTATC
GCAGCCAGAGAGAGACCTGCTGTGCTAGGCAAAAGGCAACTGCGAACCTGAATGGTCCCCTATAGAACCTGAATGCAAA
TGTTCCTGTGAGTTCTGAGTGGAGGGCGGATATGGCCCTGGCGCAGGGGCAGGGGAGGGAGGGGGGACAGGCAGACAAC
CTGGGTTGAATGGCTTTGAGTCACAGACTAGAGGGGTAGATTCTAGGCTTCACCATCCAAACTTCACCATCCTGAGTGA
GAAGTTGTATGAGGGATTATGCTGTACTAGACAAGGGATGGAGGACCAGTTAAGATGCTACAGCAAGCCGAGAAAGGAT
CGTTGATACCTCCAGGCCTTCCTGTGGCTTTCAAAGAAGTCACCTGTGGCTCCATGGGGGCAGGTATTGTATGTCCTCT
CACAGATCTTACCCAAGACATACATGCAAAGATCTCAGTCACCCCAGCTCCATATCCCTGAGCTCCTAAACTAGTATGA
GAATTGGTGGCTCCCAAATGCCAGGAGGTCCACTCTCAGTTGCTCTCGCACAGGGGCCGACACCTGTACTTTATGAGGA
AGCCCACGGACATTAACAGTCACTATCCAACAGGAAACAAGCCAAGGAATCTTTAGCCTGGAATGTTTTTGCGAACATG
CTTTAGGAGTTAGAGCTGCTGGGGTTTCTGGGAAACAGATGAGCTGGAGTTAGGAATTCAAGATGACTTAAGGAGTGAG
TGGAATCATCTGTAAATGAAAGAGGCAGAAAACAGGATTAGAGAGAGTCATTGGACTCCACAGACCTGCTAGGCTCAGT
CTGTCTCTCGTGGCCATTAGAGGAGTCCACTGTTGGATAGATGTGAAGAAGGGACTGGGCTGTTAGCAGACAGCAGGCT
TGCAATGGGCAGTCCTATTCTGGAGGCAAGCATTGACTGAAGATGTCTCTGAGCTCTGCATCTGACTAAGATGTCTTAG
TGTCTGTGTTTAAGACCCCTCTCCTGGGCTCCTGGCCAAGTAGGTCATCCCTGTGAGTTGAACCACGGCCTGTCTCCAA
CAGGGGCCATTTTCATGTTGCATCATTTTACTTTTCTCCGGCCTCAGTAGGCAGAGGCATCTCTCTAGGGGGCTGCTCT
GACCCTCCCTCCCCCTTCTTCTCAGCCCTGAGTCCTTTTTCTTTTCTTCCCCATGTGAGGGAGAATGCATATGTGTGTG
CACACATGTGCGTATGGAGATCGAGAGCAACCATGGGTGTTATTTCTCAGGCATTATCCATCTTGTTTTCAGAACGGGG
TCTCTCTCTTTGGCTTGGAATTGCCCGAGTCATTGTATAGGCTAGGATGGCAATAAGCCACAGGATTCTGCCTATCTCT
GTCTCTCTGGTTCTAGGATCACAAGCATGGTCTACCGTGCCTGTTTTTGTTGTTGTTGTTTTGTTTTTGTTTTGTTTGC
TTGTTTGTCTGTTTTGTTTTGTTTTGTTTTGTTTTTAAACATAGATTTGGGACATAATGCTTACAGTGGAAGTGCTTCA
CATACTGAGCTATATGGCCCCAGCCTTTCCTTTTATCTTTTGTTTGTCTTTTGAAAAACACTCCGCCCCAAGCCCACCA
GGAATAAAATCAAAAGGTGAACATTTGCCTCATCAAATATCTGTACCTTGAGTCCCTGGGGGCTTAGCTTCCTAAGACA
TAAACTCGGCACAAGTCACAGTCGTGAACAGGCAAAGATCCCATAGTAACTTAAAGGCCAGGAAGACCAGACTCTCAGA
TGACGAGACGCTCAATCGGACTCCCAGCAAAAGAAATCCAAGCTAAAGCTACATCGAGATAGATACCGTAACCGCCTAT
CAGATTGGCAAAGATCAAAAAGTTTGATTTGGCTCTGAATTCGCCGGGGTAACAGCAGCTCACGGGCTGGGTCAGGAGC
ATGACTTGACAAAGCCGTGTGGAAGACGATTTGGCAACCTGTCCCTACGTTCCAAATTCCCACGGTCTTCAAAAGGCAG
AAGAATGTGATAATTAAGAATGTGAGTTGGAGTCAGACACCTTGGTCTGAGTCCCAGCTCTGCCAGTCATTTCACTTCC
CCGAGCCTGGATTCTTCTCGTCTGCAAAATGGTATGAGTTCGCACAGCTCTCAAGCTGGTTTCAAGCGTTGTTATGATG
CACCGATCGTTGTAGAGTACTTACGGCAAATCCCAGCTTTCAGAAATTACTATGCGAATGTTAATGATCCTTATTATTA
ATTGCCCCAGCAATTTGACTGCTAGGAATCTACCCTACAGACACCCTTGCACGTGTGCTGACCTTCCAGTGCCACTCAG
TGAAGCACCGAGGCTGCTGCACAGAACCAATCCAGATGGCACAAGCAGCTAGCTTAACTCAGGTGTGTGTGAAATAATA
TACTCGACTGATAGGAAAGAAATGGTGAGTGAACAGTTTTTTTAATTTCTGTTCAATGTGCCACAGCACGGTGCTTTAA
ATATCTGTAAGCAGGCAGGGTAGAGAGGGCCACATGCCTATATCTTAGCACCTTGTAGATCAGGAGTACAAGGCCTGAC
TGAGCTACAAGAGACCCTGTCGCAAAAAAAATAAATAAAAGGACTGCAGATACAGCACAGAGGCGGGGTGCTTATCTAG
CACATGAAGGGATATGGGTTTAATTCCCAGCACCAGACAGCTAAAACCATCACAATAATGAATAAGAATCAGGGGAGTA
CAAACTCATGTGCATCTTATCCATAATTACAAAATTCAAAAACCTCGAATTTTTTTAAAAAAAAATGGCACAAGGATGA
TTTGTAGCAAAAGCTAAATTCAACGGACTGAAGGCTGATTATGGATTTATCTTATTGGTGCAGAAATATTAGTGCTTTA
TCAGAGTGTGGCACAAGCCACAGAACACCGCCTTCCTTCATTCCTTGAATTCCCAATTCGAAAGGACATTGCTCCCGCA
TTTTTCAGGGTTGTGGACTTATAACTTGATTTATGCCCCCTCACCCTCCCTCCCTCAGTATTAATATATCCCTGCACAG
GAAAGACTAACCCGGTTGCTCTCGTGAAGGAACTGTGAACTGGGAGGGAGCGTCTTTTGAAGTATTTCAGTCTACGGTT
TTAAAGAGTATATGGATACTTTCAAAAGGGAGAGAGCTCAGATTTAAGCCCTAGGCGCGTTCACCGGAGACCCGCCCTC
TGCCCGTGCTAGGCGTGGTAAGCTGCCTGGGAACTTGGACCTGGGAAGTAGGGCTCGAGGGTGGGGCCAGGTGGGACGC
GAGCCTCACGTCTGAGTCACAGTGCTGCCCTGGGCACCGCAGTGTGGTCGCCTCCCTAGAAAAGTGCAGATGATGAGGG
TGACTCAGGTCCCCCATCGGTTTCCTATTCTACATTTTTCCCACCTGGGGGGGGGGGTCGAAATTTGCCCCTCACACAC
CTCCTCATCACATGTCCCCAGCAGAGGCCCAGAAAAAGGGCACTGGGTCCCAGAACCAAAGAAGTCACACGGGCAAGCA
AATGAAGACAGGCTGGGCAAAGCCTGGATGTGATTTTTAAAAATATATTATTTTTATTATATTTTTAAAAGATGTATTG
ATTTTTGTTCCATGTGTATGAGTGTTTGCCTGCATGTATGTAGATTGATCATGTGGGCGCCTAGTGCCCTGGAGACCAG
AAGAAAGTGTTAGATCCAGGAGTTACAGATGTTGTGAACCCCCATGTGGGTGCTGGGAACTGAACCTGGGTCCTCCACA
AGTACAGCCAAGTGCTTTTAACTGCTGAGCCATCTCTCCAGCTGCTTTGTTTATTTTTAAAATGTTGGTCTTGCTAATT
TGCCCAGGCTAGACCTGAGTTTCTGGGTTCAAGCCATTCTCCTGCCTCAGCCTTCTGAGATTACAGGCATGGTGATACC
AAATAATGACAGGAATTAATTTTATTAACACTGAGATGATCCAAATGTGAATTGAGTGTGGCAGCCTGGGATTTATGGC
ATTTCTGTCTGATCTTTTCTTCTTCTGTGTGGGTTCTTGTGAAATGTGTGTATATCCTTCCATATTTTTCTCTAAATCT
CTCTAGATAATCATGCAGCATATATATATACATATATATATATATACACACACACATATATATACACATATATACATAT
ATGTGAGTTTATATGCATATGCAAATCTATCTGTGTACATAATGGTCCACATGTATGTGTATTTCATAACTCCGCAAGA
ATATCTGCACATATATAACCACATGTACACATGCTCACACAAATATTTATATACAAGCATATGTACAGTTATATCTTTA
AGTATGTCACAAACTCAAGAATGAACCTGTCCATTATTCATTCATAGAAAATGTTCCTCAATAAATCTCTTGAAACTCA
AATGACTGATACTTAGAGAACCAATACCAATAGGACACACATATATGACTATAGAGTTCACACAAATCTATATCAGTAT
TCTACAATAACTACAATAATAATATTGTGTGGATATTGATAGGGATTGTAGTATCGTCACAGTATTGTTCACAGTGTCC
AGAAATTATAAACAAAGCAATGTCCATTCATAAGCTATGCTCAAATAAAATGCCACGCCTCCAGGTATCAGAATGCTGT
ACAAGCCAGGCAGTGGCTGTGCACACCATTATCCCAGCACCAGGGAGGCAGGTGCAGGAAGGTCCCTGAGAACCAGGAC
AGCCAGGACTACATAGAGAAACGCTATCTGGAACTCCCTTCCTCCAAAAAAGGCACTTACTCCCTGTTTTTTGAAGACC
CAGGTTCAGTTTCCAGCACTTATGTGGTGACTCAACCATCTGTAACTCCAGTTCCAGGTGACCTGACCCCCTATGGCCT
CCATGAGCTTCCTGTACAAATGCACACACACACACACACAATTTTAGAAGAAGAAACTGTCAACACCGTTAAAAAAAAA
TGAAAAGGGGGCTTAGCGGTGACGAGCACTGTCTGCTCTGTCAGAAGACCCAGGTTTGAGTTCCAGCCCTCACATGGCT
GCTCACAAACATCTGTAATTTCAGTTCCTGGGGATCTGATGCCCCCTTCTGACCTCCGTGGACACCAGGAGCACATGTG
GTACACAAACATACATGCGGGTAAAAGATCGACACAGATACAAATAAATAAATTACTTAAATAAAACAAATAAATAATT
TAAGAAACGAATGAAAGAATTAAACTAGTGATGGATTTTTGTGGGTTTTTTTTTCAGAGCATTTTTGTTTGGTTTGGTT
TGGTTTTGCTTTGTTTTTGTTTTTCGAGACAGCATTTCTCTGTATAACCCTGGCTGTCCTGGAACTCACTTTGTAGACC
AGGCTGGCTTTGAACTCAGAAATCTGCCTGCCTCTGCCTCTCTCTCTGCCTCTCTGCCTCTCTGCCTCTCTGCCTCTCT
GCCTCTCTGCCTCTCGGCCTCTCGGCCTCTCGGCCTCTCGGCCTCTCGGCCTCTCGGCCTCTCGGCCTCTCGGCCTCGG
CCTCGGCCTCGGCCTCTGCCTCTGCCTCTGCCTCTGCCTCTGCCTCTGCTGGAATTAAAGGTGTGCGCCACCATGCCTG
ACTTTTCCAGAGTATTCTTTTTTTTTTTTTGACAGCTCCATACATGTATGTATACAGTGTATGTTTGAACACAGGACTA
TTGATAGATTTTCATTGTATTAATGTATTCTGTAATGTAGCAATCCATTATTAGTATTATTTGTATTAATGCATAAGAA
AAGATGTGCACAGTATTTAAGTAAGCCATTGATTTTAGAGATTTATTTTAATTATGTATGTGTGTGTGTAGTGTTTGTA
GGGGTATGTGCACTCATGTGCAGTACCTAAGAAACCTAGAGTTACAGGCAGCTGTGAGTTATGGTAAATTATGCTAGGA
ACTGAACTCAGGTCCTCTGCAAGAAAAGTATGTGCCCTTAAGCACTGAGCCATTTCTCCAGCTCTTAAGCCATTTGTTT
TACCTAACAGTTTGAACCGAGTGCTATTGAAAGGGTCTAAAACCGTCTACTTCCATAAGCATTGCATCCGAACTCCCCT
TAGCCACAAATGCATCAGTTGCCATAGCAATAGGTATTACATTTCTTTTTGTTTTAAAAAGAATTGTTACATTTTATTT
ATTTTGTGTGCATGCCTGGCATGCATGTGGTGTGTGCTTGCCACGGCATGCCTGTGGAGAAACTTGCTGGGTCAGTTTC
TCTTTCCACCGTATGGCTCTCTGGGATTGAACTCTGGTCTCAAGACTAGCAGCAAGCACCTTTGCAGGCTGAGCCATCT
CGTTGGCCCTGATTGATTTGCTACCGTTCTCTTTCCATGTATCAGTTTAGTTGTACACAAACACGGTGGACCTGGTTCT
CCTTGAACTTGCTTTGTAGCTGAGAAAGAACCTGACCCTCCCGGCTTGGCTTTCCAAGAGCCAGGGTTGCAGATGTGAG
ACTCTGTGCCCTGCCTTATTATTATTATTATTATTATTATTATTATTACTATTATTAATGTTGACTTAATCACCGTAGT
ACTAGTAGTTTCTCATAAATTTTTTGGTTCTCTGTATGGGTCTTGTATCAGTTGCCAATATTTCTACTGAGTCATTTAT
TTTTCCAATTTTTAGATATTCATGCTTACATAGTCATCAACTTTTAAATGTGTGTGTGTGTGTGTGTGTGCCTTTTGTC
CTCTGATTTGCAAATACTTCCCAATTTTTTTTTTGTTTGTTGCCTTTGTGCTACATTTTTCACACAGAGTTCAACAACT
CTGATGTAGTTAAATGTTGTTCTTTTTCTCTCTCTCTCTCTTTTCCAAGGCACTGGTTTTTGCCAAGGCTTCTCCCTAC
TCTGTCTTCATGGTTCATGAAGGTTACTAGAACTCTAGCTTCACATGTGCATCACAGATCACAGGCAGAGAGAGAGAGA
GAGAGAGAGAGAGTAGAAGAGGATCCCGCAGGCCTTTAAAGCAGTGCCTCTCAACCCTGAGGGTCGAGTCCCCTTTGGA
GGTTGAACGACTATTTCATAAGGGCCACATATCAGATATCCTGAATATCAAATATTTACATTCTGATGCACAACAGTAG
CAAAATTACAGTTATGAAGTAGTAACAAAAATAACTATGGTTGAGGGTCACCACCACGCGAAGAACTGTGTTATAAGGG
TCACAACATTAGGGAGGTTAAGAAGCTCTGCCTTAAAGGGATCTTTCCAGAACCTTTGCATAGCAAATTCTTCAGAGCC
TTAGTTCGCTGCACGGCTGGCATGGCATTGTTGAGCAGTGCAGTCTCATGCATTCACATACCAGAACACCCTTACCTCT
TGGCATTCAGAGGACAGGAGAGAACACACACAGCTGGGGTGATCTGGAGCCATACAACCCACCTCTCAGGACAGACAGA
GCCTCTCTTCTGCCTACACAGAGCTCCCACTGGGGAGAGGTTTCAGCTCAGAGACCACATTGGTCAAAAATCTCAGAAT
GGTTGTAGTTTCAAATGGGGATGCCTCTGCCTCCCGCTGTTCTCTCTGTGGCCTCCCCAGGGTGAAGGTTTGTGGCTGG
GTGGGATAGCTAAGCCTACTGGCTCACAGGTTGAAGGTTCTGGACTGTGGGGGAATATACCACTAGGCATTTCCAGGCA
GATGGAGGCTTGAGGGAATCGCTGTGATCCATGCCAATCTGGTGAGCGTGTCTGTGTGGACAAAAAACAACATTGGAGA
ATCCTTTCTAAGTGGGTGCTTTCCCTCCCTAGCCACCCACGAGCCTGTTTGTCTTTGACAAGGGTCCAGATCAGGGCCA
AGTACATCTACCTGGGGTCAGCTTGATAAAGGCAGCAGGGAAAGGTCGATCCAATGAAGGGTAGGGGCCATGAAGGAAA
GAAATTGAGAAGGTGTGGCTTCCACCCAATGCAAGCAAGGCGCACTGACTGTGCTGCACTTGGATTCTTTGACAGGCCT
GCATTCCTGCCCAGCTCTACCAGTGGTGGCACAAGCCTTTAATCCTAGCACTCAGGAGGTAGAGGCAGGCAGGCAGATC
TCTGAGTTTGAGGACAGCCTTTTTTACAGAATGAGTTTCAGGACAGACAGGGCTACACAGAGAAACCTTCTCAACCTTG
CCCCTCCCAAACAAAACAAAACAAAACAAAACAGAAGAAGAGGAGAAGGAGACAAGAAGGAGGAGGAGGATGAAGAAGA
GGAGGAGGAGGAAGTAGAGGAGGAAGAAGAAGAAGAGAAGAAGGAGAAGGGGAGGGGGAGGGGGAGGGGAGAGGGGGAG
GGGGAGGGAGAGGGGGAGGGGGAGGGGGAGAGGGAGAGGGAGAGGGAGAGGGAGAGGGAGAGGGAGAGGGAGAGGGAGA
GGGCGAGGGAGGAGAAGGAGAAGGAGAAGAAGAAGAAGAAGAAGAAGAAGAAGAAGAAGAAGAAGAAGAAGAAGAAGAA
GAAGAAGAAGAAGAAGAAGAAGAAGAAAGAAGAAAGAAGAAAGAAGAAAGAAGAAAGACAAAGACCTATCTGGCTGGAC
TTTATCCATCCCTAATCACTCAGAAGGCCACTTGTCTTTCAACAAAGACCCAGACAAAGGCTATCTACCTAAGATTGGC
TTGGTAAAAGTCGTGGGGGAAAAGGTGGTCCAATGGAGGGCAAAGGTCATGAAGGGAAGGCACTAAGAGGGCATGGCTT
TCACCAGCTCTACATTCTCTGCTCTACCACACACTGTTGGGATGCTCGCTTGTTTTTAATCTGTCCAGTTTGGCTCTGT
GCGCCCAGTTCCATGGACTCTGCTTGGCACCTGGTAGGCACCCCAATAGTATTTGTGGGATTGGTGAACAAATACACAG
GTGTAAGAAAGTCTCAGACATTATTCAGATCCTTGTGTGGGCAGAGTTTTGCACACATCTAAGGAGTGATCAGGGATCA
GGATAGAAGGGAGAGAAGCAGAAGCCACAGTGACAACCTAGAAAAGGCCACAGCCTGGGCCCTATGGTGAGCAGGGTCA
GCCTCTCTAGTAGACCAGGGCTATCTGCTCAACGTAAGCGATACCCAGGTATACCTAGAAGCACTTTTGTACGTCCCCA
TCATTCCCCCGAAATGAACCCATCACTACTGCCCTGCCCTGGACAGTTGGCACCAAAACCCTGCCACGTCCCCACTATG
GGACTGGCAAGGCAACACACTCATGCAGAGGCACACACACCCAGCCAGAGATCCTCCCCCAAGAGGATGACTCAGAACC
ACTGGAAGACTGGGGTCCGTTAACCAGGCCACCTCTCTTCCTGGGCATCCATCCTGGGCTCCCGATGTGGACCTGCCTC
TTCACAGCCTGGACAGTAAGGGATTTTCCCAACCCCAGCCCGCTGACATGTGGAATCTTAAAATAACAAAGCCAGACTC
CAAGAGAGGAAATTCTAAGAACATGAAATAAGTCAGGAGAGCAGAGGTGGGTGTTTATTTTCCATGACCACCTACTCCT
CTGTGCCTGGGCTCCCAGGCTGGGGGAGGGGTCCGACGAGAGTGCCACAAAGCTAGAGGAAACCCTTCTTCCTGTGTTT
GCAGAGGAACAATGCTGGAGGCGCCCTTCCAAGGCCTGCAACTTTTCCTCTGGACAGAGGATTGTAGGTCTCCACGCAT
TCCTAGGAGGAGGTCCCGCAAGGAATGTCGAATATAAACATTGACTTTTCCATCACTCAGTAAAATCCGGACACACAAA
AGTCACAATCCCCTGGAGTCCTTCTTTTACTCAGGTTCACCGCCCCAGGTAGGCATGGGAGAGCCCCGCCCTGCAGCTG
GATCCCTGAGAGCATTGACCTCATTGGTTTCCTCTGTCCCTACCTCCCAGCGATGCCTTCTGCCTATGCCTCTGCTTCT
GCCCACCCCTTCCCTGGACCCTTGCCACCTCCAGCCTTCCTGGCCCCAGTGCACATTGCTTCATGCATCCTGTGTCTAG
CCCATGAACCATCCTCCATACTGTACCTCCGAGCCCGGCTCCTTTCCCTTCTAGAACTCCCCATCTGCGTGTATTCTGG
GACCTTTCTAAGAACCTGGTCACACACTAGTCTCCTCTCCTGAACACAACCACCCCAAACCAGGAAGTGACAAACAAAA
CGCCCTGCCCCGCCCCCAGCCTGCCTTTTTCTCAGGCAGTTTACAGAAACCAAACTGCTTAATGGGGCAAGTCCTAAGA
CACTGGCCAGTACTTGCCAAGCACCTACCAGTGAGGGTGCCATGCTCAAAGGGCATCAAAACCCCCGAAGCAGTGTACT
GGCTCCCAGTCCTCACGCAGAGCCAGAGCTCCATTAACTAGTCAACACCATGCGTTGGCAGAGAGCACACTCCATCTTC
TCTGTAGTTCAGCTGAGCATTTTGGCAGGCCCTTTGATGGCCACCGACACCACGTGCTTTCTCTCCATCTGCAAGATTT
CCCTTCGTCTAGAACCCTAACTCCCATTGGTCTTTCAGGTTACTCTCCAGGATCTCCTAAGGGTTCTTCCTGCATCTTT
GCCCCCTAATCTTGGTTATTCATATATCTATATATCTATATATCTATATATCATCTATATATTATCTATATATCTGTAT
ATCTATATATCTATATATCTATCTCTATACACACACACACAAACACACACAGCCCATATCACAGTGTATCACAGGCTGC
CACAATCAGGGTCCCAGTCCCTTGGGGAGAGTAGATGTTTGTACCTCTCTGTCCCCAGAATTGAGCTCAGGCTCTGGCT
CAGAGAAGAGACTTAGTAAATATTTGTGGACTGAATTAAAGCCCTACCCTTGGGTATTCAGTCACCTTGGCTGCTCTGA
GAGCCTCTAATCCCTCACTGGGTCATCTGCCTTCCTTTCTCCTGTCCCAGTCTTTGCCTTACACAGCAGGTCTCCCAAA
ATGTGCAACTCTGGGGAATCTGAGCCTGTATCCCTTGAAGCTGGTAGCCCAGAAGGACAAATCCTATGCCTCCTTCCAC
GGCATGGCCATTTTCCAAATGTCCAAATATATTGCTTTGGGTTTTTCTTAAGCCATAATCGGCCTCAACACTTCCCTGC
AGGGATCTAGAGCCAGGTCCCGCTGCCCTAAACCCCCAAGGCCTCATCCTTAACTTGGATGGACAGGGCTCCGTTCTGC
AGGCTCAGTGGGGTGTCTGACCAGGTCCTGCCTGGTGTTCAACGGAGGGAGGAGGACCAGCTTGTGCATCCAGGGCGCG
CAGTGAACACCCTGTTCACATTCCTGAGCCAGCCGGAATGCTTGGACGAGCCAAAGGCCGCATTCCTCCGGCAGAGGCC
AGATCTCAACCCAGGCTCCTGCCCCGCCCCTCCTTGGCCAGGCAAGCGCAGCAGAGAAGCCCGGCCCTAGTCCGTGCGG
ACTCCCCCTGGTGGCCAGGGCTATCACTGCTCGCTTGGACTCTCCATTTGTAGTTTGTATTGGACCATGGATCACTGCC
TTCCTTCTGCCGGAAGCTTCCAGATTCCAAGCTGTGTGATTCCCATTTGTAGCTCCAACGTTTGCCGTTGTGACCTATT
CTAATATCCTGGTACCCTCCGCCCATTCCCCACCCCTGGCATCCTCTCAGGAGGAATCACCGCCCAGTGTGTCACCTTC
CTGTAGGTACTTTCAAGGCTAGATGAGTGAGTGGCGGGCCTCACCTTGGCCCAGTTACTCAGTGCCCACAGAGTGGCTT
TTCTGAGACTCTCGTAAGGTCTTAAGGAAATGTAGTCGGTCCCTTTTCTTGGTGGCCCTCGGCACTGTGAGCAGACTCA
CATATCAGGGCTGTGTGGATAGGAGGGTTGAAAGTTGAGAGTTAGCTTCCAGGATGCTACCAGCACAGTAATACGAATA
TGCAACTCTCCCAATATCTAAACGCTGCTCAAAGAAGTCAGCAAAGGATTTTTTTTTTTCTTAACCTAAAGCCAGGATT
AGGCAGTGCATGCTCAGCCTAAGGGAGAGGGATGTACAAGGCTCACGTACTTCTCATGGAGGGACCTGCCTACATTCCT
GGTCAGTAGGGACTTTGGGCAATGGGGCAAGACATGGCTTGTGTTTAAATTGATGGCACATGCCTTTGAGCCCAACACT
CAGGAGGCAAATGCAGGCGGATCTCTATAAGTTCGAGGCCAGCCTGATCTACAGGAACAAGTTCCAGGACAGCCAAGGC
TATATAGAAATACGGTCTTGAAAGAAAGAAAGAAAGAAAGAAAGAAAGAAAGAAAGAAAGAAAGAAAGAAAGGAAGGAA
GGAAGGAAGAAAGGAAGAAAGGAAGAAAGAAAGGAAGAAAGAAAGTCAACCATTCACTACTTTGACCTCTTACAAATGG
AGCCTTGCTGTGTTTGTCTTCATACCATAGCATGAGTTCTTATGGAGCCTTGGTTGGCCTGGAACTCACAAAGATCCTC
CTGCCTCTGTTTGAGTGCTGAGATTAAATACATTTTCTTCTTCATGTGTTTAAAATCTTTTCCACTTTCCCAGATTGTT
GACCTATCTTTCTTTGGAGGAAAGGAACAATAGATGAACACCATCTACATAGGAACACAGACATGGTTATCTGCCTGCA
CACCTGCTGACGTACCTAGGAACCCAGCAACATAGCACAGCCTGAAGCTTGCATTGCTCAGGAGGGTGGATCCAGGAAA
GGCGAGAGAAAGAGCAAGAACCAAACTCTCATTGAGCAGCTTTTCAGCTTCTAGAAAGGGTGGGGGCATAGGAGGACTC
TGTTCAAGGTCGAGTGTGGTGGCTAGTTTGTCTTGTGTGGTTGCTTTCCCCGCCTCTTCAAGCAGGAAGATAAAGGGGC
TCTTTAGTTGCTGGCATAGGTCTTTTCCATCATGTCAACATGGAGACCCATAGTGACAGGGTGGAAAGAGGCCATGAAG
CCCTGGGTTTGAGAGTGCAGCCCTGACTCAAGCTTCTCCATCTTTATCCCCATCTACTCTTCTCAGGGTGTCCTGGTGG
GTCATCAAACGGACACCAGGGCGATGCTTCAGTGGGATGTGAGGATGCGTGATGTGATAATGACTAAGTCAGACTCAAA
AAAAACCACCATTTCTCAACTACTTACCAACCTTGGGCCCTATTCCCAAGAATCTCAGTATTCCTTGGAGGTGACAGTG
TGTTAAGACAGAGGCAGGTCCAGGCACAGCTGAGGCACAGATGGGTGTGTTAGAGTCACCTACAGCAGAGAAGAACATT
GGGGTGGGGGGAGCGCGGCCTAGAAAGGACTTTAGTGGAATCCTGGGAGATATAGGACCATAGAAGCTTCTCCCAGAAA
GGGGGGGGGGATCTTTCTAGAGTCTTCTTTCATACCCTGAGGCCACCATGTCCCTGTGCTAGTATTTTGCTAGAGAATT
CCTGTCATTTCTGCTACTCATGTGTCTAGATCAGCTAGCCTGTAAGCCTGTGCTCTAAAGCCACTTTCTGAAGATGGAT
ATGATCAAAGATTCCCTAGGCAAGGGAGTGGTTTCAGGAAAGAATTAGTCTCAGAGTGTTGGCCTCTTCCTCCTGGTAA
TGCTTCCTTCTGCCCGGAGCCTTCTGCATGTTAACCTTCAGACCTCTGTGTGTCCAGGGGGCTGCATGGAAGGCAATGG
GCAGGGCCCAGTCTCATAAGCAGGGTGAGTCTCCTTCTGCACAGCTCCACTCCATCGGCCCTTACCTTTCCTAATTAGG
TGGATGTCTGGCCTCCTTGTGCTTAGAATGAATAGAAAATTACCTGGCTTGACTAATTTCATAGGAAAAATAAGGCCTT
GTAAAAAGCCATTTGGTGTATATTGTTTTGTCTGCATGTATATCTGTGCACCACATGTGTGCCTGGTCCCATCAGAGGC
CCAAGGACTGAAGATACAAATGGATATGAGCTGCCAGGTAGGTGCTGGGAATCAAACTTGGGTCCTCTGGAAGAGGAGC
CTGTGCTCCTAACTGCTGAGCCATCTCCCCAGCTCCCAGATAAGAAGCTGTTTACAAGTTTTTCTTCTGGTTCTATTCT
TTATTTCTTTCACATTTATTGGCTTATTTTGTGTGCATGCGTGTGTGCCTGCATGTGTGTGCTTGACGTGGCATTGTGT
GGAGGTCAGAGGGCACCTGTAGGGAGCTGGTTCTCTTCTACCATGTGAGTCCCAAGGATCCAACTCCTCGGGTCATTAG
GCTTGGTGGCAAGTGACATTAATCACTGAACCATCTTACTGGTCTTTCGTTATTCTACATACTTTAGATGGAGTCCTTA
ACTCCTGATTCTTCTTCCTCCCAAGGGCTGGAATTACAGGCACGCACCATTCCATCCGGTTAATCTTGGAGATTCAGCC
TGGAGCTTCCTGCGTGGTAGGCAAATGAACTCACTGAGACGAACTTTGTCTTTGCAAAGTTTTAAAATTTATCTTGTAA
TGTTTGGAGTTGGGGGAGATGGACTGGGTTTGTCGATGTGCCTCCTGGTCCCACTCAGAGATTCTGAGTCAGGCAGTCT
TGAGTAGGGTCCAGGACTCTGCATATTTTGAGACTCATCCCCAAGTAATCCTGCTGCTGCTAGCTTGGACAGGAATGAT
AAAGGTGAGTTTGCATTCCTTCTCAGCAAATCCACCCAGTGTGAACTTCTCCTTTGTCAAGACTTGGACAAATCCCACA
AGATTACTTTTTAAATCCGCTGTCCCTAAAAAAAGAATTTCACGGAGGAATGTAGTTTGGAGGAAGGCCCTCCCACTGA
ACAGGGATAGGAAGTGCACGGTAAACTAGGGTAGAGCTGGAAAAGCCCCACCAAGCAAGCCGAAGAACATGCTCGGGTG
AACACGCTCGGGTGAACATGTGTCGACTCCTTTTCTTCAAGCCTCTTCCTTTCTGTACCAGGTGCCAGACGGTCCCGAG
GCAGAACAAAGCCACAGAAGACAGCTGATATGAAAGGCTTTAGCCTTCATAGAACCAAAGTCCCCTTGGGTCATGGTTC
GAGTGTCTGTACCTTGACTGCCAGGAACAGTCTTCAGAGTCCAGTTACCCGGGTCCTTGGCCCCAGACAAACTCCAAGA
GTTGATAAGATAGGAAGGTCGTCTTCTTGGCTGTGGCACACACTTCCTCAGCCCTGGCCTGATTTTGCTTGCATCCCCG
CTGTGACATGAGCAAGCACGCATGTCACATGTCACGAGCTGCTGTTAAGGTCAAGGCTCGGGGACCTTTGATATTACCT
CAAGCTCATCAGAAATGAGAGACCTGCTATTGTTTCAAAAGGTTTGACAAGCTTGAATTTGGATGGTACACCTTGTCAG
CACTGTCCTCGGTTCAGTGTCATTGCGTAGCTGAGAGGTCGGACAGGCACGGGAGTGATATCTTAGGCATTTTTCCAAG
CTCGAATGCAGGTACTGGCGACACTGCATTCGGGCCCTCATTTCTGAAGATGCTTAAACACCCCTCCCCCACTTCCTTC
CGTGAGGACTCCTTTAGTTACTTTGTGACACCCAGTCCTTCCAGTAAGTAGAAAGGGTGTATGCAATAGAATTCTGGTT
ACAGGGAGGTGACTCCACCGTCCTTCCACGCTCACTATATAGCATGCACTCTGCTGACCTTGTGACACAGGACCTCAGA
AAGCATCAGTGCCAGTCTGGATAGCAAAGGTGTTTTGTTTTTTGAGACGGGGGTGGGGGGGGTGGGGTGGGGGGTGTGC
CTCTCACATTTCTATCTCTAAAACTAACACAGTAAATACGTAAGTAAATGTTATACAATGTTCTTTCTCTTGCCCTTAA
CAGATCATTTTGCATACCCCCCAATTTGGAGATCATTGACATGGCTGGAATCTTCCTAGCCAAGGACACACATCTCTGA
GAGCCCGCATTTCTCTCTCAAGAAGGGAACACACACACACACACACACACACACACACACACACACGTATTTAAAAAAA
AACCAAAAACAAACAAACAAACAAAACACCTCAAAACCATTTGAGTAGGTCTCTACAACACAAAGAAACTCTGTCCTAG
GAGAAAAAGTCGTGCTTCTACTTCTGGATCTGGGAAGCCACACTCCCATTATTCTGGGTCGGGTAGGTCCCAGACTGGG
GGAAGGGAAGGTGGAGAGGGCACCAGGGCGTCTGAATTTGTGGAATTCCTTTGGACTCACTGGCAGTTCTCAAACTGCG
TTCCTGGAGGGATAGTGGCTGTGTTGTTGGCCTGAGCATGGGGAAACAGCGGCCCCTGGTGGTGAAGTTATGAAATGCT
CTCCAGGCACCGATTAGTAAGTCTTGAGTTTGTCTGGGGCCAAGTAAGTCTTAGTAAGGCTATCACGAGACATCTTGTC
CTTCCAAACTCTGTAAGCACTTTCAGAGAGGCTCAAATGTCCTGAAAGATTAAAAAATGAGGGAACCATTTGTTGCTGT
GCTCCCCGGTGGCTATCCTCGTAGCCTTGAGAGGTGTCGGAGCTTTTGAGGTTACTAATGTCAGCTACTCTGTGCCAGG
AAGTGTTCACACAATCAGGCTGGAAGGTGTCCATGGTGAGGCTAGGGGCTCCTGCCAGCTTGAAGCCCCTCCTCCTCAT
GCCCCCTTCCTCCCTAGGGATTCTTTGCTCCCCCACCCATTGCCTCTCACCCTCTCATTTAGCTGAGCTGGTTTCTTGT
TTTTGTAATAGTGGGTCTTGAACCCAAGTCCTCACACATGTTAGGCGAGCACTCTGGCATTGAGCTATGCCTCCTGCCC
CCCCCCCCAAACTCACATTTAGCTTGCTTATCTCTCCACTAAAGCAGAAACCATCAGGACTCACATAGCCTTGCACTGA
AATACAAGAGGACTACATAATTGCTCCTGCTTGGTTTCTGTTTTTTTGGTAGCTTGGAAGACAAACCCGCTACACTGTG
ACCATTTTCTTTTGGAAAGGTCTGATGTAGCCATTCCGCTCCGGCATGGACTGGGGTAGAGAATAGGCAAAGAACCACC
CATTCCAAAAAAAGAAAACATGATTTCCAAAGAAATGCCCTGAAGCCAATTGATCTCCAAAGCCCATCTCCTTGGATGC
TTGTGAATGCTTTTCCTTTTAGGGTTAGAGGACTGAAAACGGCTCACCCTTTCCCTGCCTTTTTCCTCCCAGGGACATG
AGGACTGTCTCTCTTGGCTCCGACCTTTCAGGGAGGAGAGACGCAAAGGCTGTGGTAAACCCATGGACAAAGGATTTTT
ATGAGCTCAAAGGTGTTTCCAATTTCTTTTCTTCTTATTTTTTTAAGATTTATTTATTATTATACATAAGTACACTGTA
GCTGTCTTCAGACGAGCCAGAAGAGGGCATCAGATCTCATCATGGGTAGTTGTGAGCCACCATGTGGTTGCTGGGATTT
GAACTCAGGACCTTTGGAAGAACAGTCAATGCTCTTAACCACTGAACCATCTTGCCAACCCTCCAATTTCTTTTAGACT
AGACATTAAACACCATTAACTCCAGGACAAGTGCAGGTACAGACACCATAATGGGCTAGTGATTCCACTAAGTATGTCA
TGTATTTATGTTGAAAAAAAAGGGGGGGGATAATTTGGCTTAGCGGTGGTGGTGGCCTTTAATGCCAGCACTTTGGAGG
TAGAGGCAGGCAGATCTCTGTGAGTTCAAGGCCAGCTTGGTCTACAGAGGGAATTGCAGGATAGCCAGGCCTACACTAA
GAAACAGAACCATCCCACTCCACCCCCAAAAGAAAAAAGTGTAATAGTTTAGCCAAAACTTGGAGACAAAACTATCCCA
GTAGGACCAAGGGACCAGCCGTGAGATAAGGAAGAGGTGTGAATGCTAACTCTTTCCCTCTTTCTTCCTTTCTTTTTAA
AGATTTATTCATTTACTTTATGCATATAGGTGCTCTATCTGCTTGTACACTTGCATACCAGAAGAGGGCATCGAGATCT
CATTACAGACCGTTGTGAGCCACCATGTGGTTGCTGAGAATTGAACTCAGGGCCCCTGGAAGAGCAGCCAGTGCTCTAA
ACTGCTGAGCCATCTCTCCAGCCCCTCAACCCTTATTTTCTAGTAACAGAAGCTAAGAGATAACATGTGGGGGTCTGGA
GAGTCAATTGTGACCAAGCCTGGTGACTTGAGTTTGGTCACCATGAGCTGTATTTACTTTAATTATTAGAAAATGAGCT
TTTCAGATACTTCTTTGATTTTTTTTCCAAACATAATAATTTAGAATCTTAAAGAAACCAGTAAAACGTGTCAATGTTG
ATAGACATTTTCAGAATAAAAAGACCCAAAGCTGGGAGGTGAGGGCAGGAGATGAAGAAGACTCAGACCAGTCCAGACT
CTAAGCTAGGGCGAACATGTGAAACTCATTGTGAATGTATCCTTTATTCAGCTAGCTAAGGTAATTGCCATCTAGGGGG
TGTACTGCTTCTGTGTGCTAACCTAGGCCTGCTCCTGTAAGCTTCTAGCCTCTATACAATCTTCTTACCTAGGCCTATA
ATGTTTTTGGCCTCTGAGACTTGCTGCTGAATAAGTTCACCCTTTCTTCTTGTAACTGAGCTCATATACAGACCAGTTC
AACTCAGCTGTACTGGCTCAAATGCCTCTCCAAGCTGACTGATTCAATCTGGCTTCTCTCAGTTTCTCCTGACTTGCTC
TGCTTGGCATCAAACAAACTCTAGCAATCTGTCCTGATCTTCTGGCTCCTTCTCACTCTTTCTCTAACCTGTCTCTGTA
CAACTGTCCCAGTAAAACTGTCTTCTTTTCTCCCGTTCTCTCGGACGCTCTCTTTTAAGGAGCTCCCCTTTCCTGTCTT
CTCCTGAGAGTTGGGCACATCCTATTCTGTCAAATCTTTCTCTGATTCGTCACTTTGTCTGCCACTCAATTAGACATCA
CTTTCAAACATGGGTGCTCCTTTCTACAATCTAATCTTACCTTTACTGGTTGGAATTAAAGGTGTTTATTAAGGGCATA
TCTGTGTTCTAGCCAGAGGGATTAAAAATGTGTGTCAAGGGCTGAGCCATATCACAACTAGGAATAGGTTTTTAAGTAA
ATAACCATCCTGGGTCCACAGTGTGATCAAATATCCTGCACCAATGTATACTTTGTGATTCTACTTATTTTATTTTTAA
AATTTATTTCTTCGCTATGTATACATTTATCTGCATGGATTTATGTGCTGTGCACCATGTGTGCTGCACATGTGTGCAG
GTGCTGGTGAGGCCATTGGATCCCCTGGAACTGGAGCTGCAAGCAATTGTGAGCTGTCTGATGTGGGAGGAGGAACCCA
ACCCAGGTCAATGGAAGAGCAGTCAGTGCTCTTAATCACGAAACCATCTCTGCAGCTACAGTGGGAAAAGTGAGCAATG
CACACCAGTGTAAACATCTTCACCTGCCTAGATGCCCACAGAACCAGAGAGCCCAGTGCTGCCTGTGGGGAGGGGCTGT
GCATGAAGAGTCAGAAGAGCTGTGTCCTTCATCTCAGACCAGAGGAATCCTTTCACTCTTTTCAAGGTAACAACATGAA
GAACATAAAATGACATCCTCCAGATGGTGGTACTTTGAGTTCATCATAATTTTCTTAAAATCATCTGCACTAAGCAAAA
AAGAAAAGAAAAGTCTTAGCCTTGTGTTTTATTTATTTATTTTTATACTTTTTGTTTGTTTGTTTGTTTTTCGAAACAG
GGTTTCTCTGTGTAGCCTTGGCTGTCTTGGCACTCACTTTGTAGACCAGGCTGGCCTCTAACTCAGAAATCCGCCTGCC
TCTGCCTCCCAAGTGCTGGGATTAAAGGCATGCACCACCACTGCCTGGCTTTTATACTTTTTAAATTACATTGATTTAT
TTACTTCGTGTTGGGGGAGATGTGCATGTGTCATGACTTATGATTGAAGGCCACAGAGCAACTTGAGGGACTTGGTTCT
CTCTTTCCACCATGTGGGTCCTGGGAATTGAACTCAGGTCACCATGCTTCAAGCTCCCTCCTCCACTGAGCCATCTTGC
TGGCCCCAGGCTAGTGTGGTTTTAAGGGTCCTCTGAGTGCAACAGAGACTAGTACAGAACACTCTTGTGTATCTGGGTG
AGATTTCAGAACTCAACGGGGAATGTGCACACGATTCCAGGAAACAAACAAATAAACAAACAGGTCAAATACATATACG
AGAGTGAGAGCCACCTTGATGACAAATTTTGTAACAGTAGGCTGCTACAGACAACAAAGCACAGTTTTCCAAGTTTTGG
AAGAAACTTAAATTGAATCTGAAACTTGATAGTCAAATTTCTAACCATCACGAAGAGCAAATAAATTTTCTGAAACCCC
ATGAGTCAGACAATCTACCCTCTCCTTGCTTATGAAGAAATTACTCCAGGGGCTGGAGAGGGGTGAAGAGCCCTGGCTG
CTCTTGCAGAGGACCCCAGCTCTACTCCCAGCATCCACATGCAGGCTCACAACCATCCATAACTCCAGTTCCAGGGCAT
CCAATACCTTCTGACCTCAGCAGGCGCCAGGCACACAAATGGCACACAGAATAAAACATTAAAACAACCCATTCAAAAA
AGAACAGCCCAAGAGTTCGTAGATGGAAGCTGTATATGAGGATAAATATTAAAGTGAGAACAAAGATGAGTTGGTACTT
GTTTGGACTGTGGTACTTGGGATATTCTTTGATCTGCAGATACTTAATAATAACCAGGGTTGTTTGTTTGTTTGTTTTG
TTTGCCACAGGCCTTACTCTGGACTCACCATCTTCCTTTTGGAGTGCTAAGATTACCTGCATGACCACCATACTTAGTG
TCTTAGTTTGGGTTTTATCGCTATGAAGAGACACCATGACCAGGACAACTCTTATAAAAGAAATTGGAGCTGGCTTACA
GTTTCAGGTGTTTAGTTCATTAACATCATGGGGAGAAGCATGGTGGTATGCAGGCAGACATGGTGTTAGAAAAGGAAGT
GAGAGTTCTACAGATGATCCACAGGCAGCAAAAGGAGATTATGTATCATTCTGGGTGTAGCTTGAGCGTAGGAGACCTC
AGAGCCCACCCCACAGTGACACACTTCCTCTACAAGGTCTCTGTTGTGTTAGAATCACGGCTTGTCCGGACAGATCACA
GCAGGTCAGTAAGTTCCACAGGAGAGAGGTTTATTGGGGGATGGGGGAGGACAAAAGAGGGCAACAAATATGATGAGGG
AAAGAGAGCAAAGGTCAGGAGTGGTCTGCCTTTTATTTGGGTTGTGACGTAACAGCTTGCAGGTAAAGGTGGGAGGTGG
GTGGACCAGATGGACACTAGGAATGTACAGTGATTGCTATGGCAACAGATGTGCAGGTCCCTGTTGCTTAGTGACCTGC
AACTGTAAAAGCCAGTTTCTGAAGCCAACAGCCACGCCTCCTTCAACAAGGTCACACCTCCTAATAGTGCCACTCCCTC
CCGATAGGCCAAGCCTTCAAACACAGTCTGTGTGGGCCATTCCTATTCAAACAACCACATATAGCTTAAGATTGCCACC
ACCCCCCTTGTTTCACATATATGTGTGTGTGTTTGTATATGTATGCAGGTACAAGTTCATGTGTGTGTAAGTGTGTGTG
AAAATTAGAAGTTGATGTCAGACATCTTCTTCAACTGCTCTCCACCTTTTAAAAAGAATTAAGGTTTATTACATTTTCA
TTTTCATTTATGTTGGTATGGCATTTGTGTGTGTGTAGCATCCGATCTCCTGAAACTACAGCTACAGAGAGTTGTACGC
CATCAACATGGATGCTGGCATCTGAATGCTGGGACCTTCTGGAATGGCAGCAAGGGCTGTAACTGCTGAGCCATCTCAT
CTCTCTAGCCCTCTGACATCAATTCAGCTATACTCGCTGGTCTGCAAGCTAGATGTGCAAAATCGGCAAGCTCTGTGTT
CAGGGAGAGACCTTGCCTCAATAAATGAGTGGGAAGCCACTAAGGAAGAAACTCAACGTCAACTTAGAGCCTCCACATG
CACATATCACACACACACACACATGCACACGCACCAACACGCACTCAAGAAGCAAAACTGAAATGTTGATGTTGTAGGC
TGGTGAATTTCTGGATTCCCAGCATTCTGGAGGTGGAGGCAGGAGGATCATGTCGTCCTCCACCCCACTTGGAGATCAA
GGGCAGTATAGGTGAAATGAAATCCTGCCCCCCAAAACAAACACAAAACAAGGTAACGTGACACACATTTCTTTTGCTC
TGATAATCTACAAAGTGAACTAGAAACATTCCTTAATTTTGTATGTAAGTTAAATGCTCTTATATTTCCATTTAAAACT
GGCAGTGCAGAATATAATGATGAATGGTGGAATTCATACTAACTGGAAAATTTTCATTTTTCCTTCCTGAGAATGACAT
TAAATAGCAACTAAAACGCCACAATAAATCATGAAGAAAGAAAAACATTCATAGTTCAGGATCTTTGGTAGTGTTTTCT
GCATTCTGAGCAAGGAGCCCGGCACTTTGTATTTTGTACAGGGTCCTGCCCGCCTGGCAGCCCAGGGTGACGGAAAGCT
GCTTCAGGATTCTACACAGGAGGTTCACCTCCCGGGACCTTGGCAAGGCACATGTCTTCTGCCCACAGTGGGGGCCAGA
GGCCCCGATGTACAAGGCTCCTGTCACATAAGGCCCTAGGCACTGAGGATCCTCTGCATATACAGCCCTCAGTGGCTTA
GGAAGACACAGTGAGGTCTCTCTTTTAGCAAATTCCCAAACTGGCATCTTGGGGAGTAGAAGAGAGAATGTGGAGATGC
TGAGTGTTCCAAAGACCCCATGGAAAGTAGCCAAACAAGGGCGGTGTTCTACTCCAGACAGACCGGAGGGGAGGGCAGG
CTGCTGGAGACTGTTCTAAAAAAAGGGGGTGTATATGGCTAAAAGGCAAGCCTAGGCTTCCCTGCTACCCTCTGGGAGG
ACCACAGACAATGCTCTGAATGGGAGATGCCTGTCTGCAAGTCAGCTGCCTTTAGATTTGTGTAAATTTTGTCTCAGAT
AAACTTCTCCCAGGCATGTCTAGAATCAAAACCAAAAACCAAAACGAGGTGACATGATGAGATGTTTCTATGTGTTTTG
ATGAAAACTCAATAGGCATATGATGATGACCTATGAAACAAACAAACAAAAGCTAACAAAAAACAACACAACTTGGAAG
GTGAGGGCTAGAGGACCAGTTCAAGGCCAGCCGTGCTGACATAAGAGCAACAGTGGCACATGCCTTTAACCCCAGCACT
TGGGAAGCAGAGGCAGTCAGATATCTGAGTTTGAGGCCAGCCTGGTCTACAGAGTGAGTTCAAACCCTGTCTTGAAAAA
CTGAGGGGGAAAAAATATACCCATAAACACACAAAATATTTTATTGAGAATATCTAATTTATTAGGAAACATATGTTAA
CACAGACAGGGCTAAAGGGCCGTGTTATGCGTGTAACAACTTCATCTTACGCAGAGTTCACGTGCTGGGACTCGTGGCT
TTTTGGTGCTAACCTGGAAGGTGCTGCCCCTGATTGCCAAGAATCCTCAGCAAACTGGTTCACAACCAAAGATCCAAAC
ACTAAGATGCAGTCATAGTCGGCAGTGCTCCCCACCCCCTCCCCAGAAGGCTCTCTCCGGTGCTTCCCCTAAGAGCCCA
GCTGTTGGCACCCAGCGAAAGGACAGGGCACCAAAAAGGAAGTATCACAGCCAGAGATTTTCCTGTCACTCCACAGGGA
CTGACCGCCGTGTCTCTGAACAGGAAGGAGAGGTACTTTTTTGGGCCACTGCCCTGTGGCTCCGTTCTACCTTTGGGTG
GATTTATTCTATTTTCATGGTTTCCCTAAAATGATAGTGGTTCAAACCCACTTCCTTCCTTTATGGCAGCAGGCCCCGC
CCACATCCTCCGCACTGGCTGTTAACCTTTCTCCCTATTTTCGGTGGTACAATAAATTAACACAGCTCACATAAAGGGA
GTGGAGGGCATTGTATACATACGGAATTCGACTGGAATTCATCAACCTGCTGTCTATCTCCCTTCCCAGTCACTGCGCA
CCATAACGCACCATGCGCACTGCGTCCCTGTGCGCCATCTCTCACTTGGTAAATAAAGCACAACGCCTCTCATTTCCTT
TGTTCACGTCTCACTTTTATGCCCTAAATCTCCCCCCACCCCACCCCACCCCACCCCCACGTTTGGGTGACCAGATAAA
CGTTAGGACACTAGAGTTAACATTTTCATTTTAGATGAGCGAAGGACAATTTTAATTTTTTTAGTATAAAATATCACAA
ATATCATATATAATATATAGAATGTACTAATCCTAGCAAGTATTTATCTCTCTGAAAGTCGGATTTTTAAGTGGACATT
GTGAGGTTTTTTGTTTTTTAATATCTTGTTTCGTTGTCGCCTTGATTTTTTTTTTTTTTTTTTTTTTTTTTTTTTTGGT
TTTTCGAGACAGGGTTTCTCTGTATAGCCCTGGCTGTCCTGGAGCTCACTTTGTAGACCAGGCTGGCCTCGAACTCAGA
AATCCGCCTGCCTCTGCCTCCCGAGTGCTGGGATTAAAGGCGTGCGCCACCACGCCCGGCTGTCGCCTTGATTTTTGAA
ACATGTCTCATTGTGTAGCTCTGCTTGGCTTGGGACATGTATGTAGACCAGGCTAGCCTTAAACTTGCTGCGATTCTCC
CACATCTGCTTCCCAAGTACAAGCTCAGCTCGGTGAACTGTTATTTGCTGGTTCTCCTACAGCCGCGGTGACGTCACTT
AATAACATGTAAATTAGCTTGTATCACTCGAGTTCCAAATCCAGCACAGTTTCCCTGGCTCTGCGCACAGTCCCTTCCC
TAGATCTCCCCAGATGTGCTCTCCCAGTCCTTGCTACCCCTCGTTTCCTCCCTCCCTCCCTCCCTCCCTCCCTCCCTCT
CCACCACTTGCTCCGAGACCACAGGAGCTTCCTTGTTCTTTGCTTCTCCCAAATCTCCACTTCCTTCTCCTTCGTCTGC
TTTAACCCAGTTGATGTTACTCTCTCTGGAAAGCTTTCTCCAACACCCCTCTAAAGCCCGAAGCCTCACCACCCTTACC
CTCTCTGCGTTTTCCCTGTATCCCACCTTCCAACCAATCACCCGGTCGGTCGGAGGACAGGATCTGTCCTGTTTAAGCT
GAATCTCCAGCCTCTGGCCCTGCCTGGCAACTGCAGACTTTCAATAAGTATTTGTGAATTAATGAGAAAACCCAAGGCC
AGGAAGTGCAGCCCGGAGAAGCCCAGGAATCAAGCCTTCGCTTTGTCTGTCCTCTACAGTCCTCTGTTGGTCTAAAACT
CCTCTTACTTTTCTCCCCCAAGGCCACCCTTGGCCACTGTCCCGGGTCTAACTAGGACTTTTCTAGGCACTGTCGAGAA
TGGTAATCTAGCATCCCTTTCCTGCCCCACCTCCACCCCCTCCATGTCTCTTTCATTTCTCCCTCTCCACGAGCGAGCA
TTTATAATTTAAAAAGGAAGCGAGTGACTGGTGCTGAGGGCTCAGTCATAGAGTGCTTGCCAGACAAATGTGAGAACCT
GAATCCAGTCCCCTGGAACCCATAGAAAGGTGGGCTGTGGTGGCATAAGCTTGTAATCCCAGCAATAGGGAGAAGCTGA
CCAGCCAGCATACCTGAGTTGAAGAGTGTTCTAGGCTCCACAAAGAAGAGGTCTGTCTGCCTGAGTGTTCTCTCTCTCT
CTCTCTCTCTCTCTCTCTCTCTCACACACACACACACACACACACACACACACACACAAGTGGGGGATGGTAACTGAGG
AGCAACACCTAAAGTTGGCCTTTGACCTCTAAACACAGGTGCATGTACCCCCCCAACACATACACAAACTAAGTGAAAA
GACTTCACTTTGTAGCCGCTGAGACTGAAGAGTGGTGGTTGGGAGGCTTTGAAAGCTGAGAACAATGCCAGTAAGCATC
CACTTCTTGCCATGTTTGGTCCCAGGCTTGGGTTCCCGGATGAGAATTACCAGCTCTCTGGGTACCAATCAGGAGAGAA
GGGACTTTGTTCTGGAAGCACAATTGCAGGCACCGAGAGCAGCCCTTCACTTTCTGTCCCCACTGGGATTCCGGTGGGA
ATTGGCAGGGGCTGCAGTTAAGTGTCTTAGACATGCAGGAAGACCTAACTGTGACCTTCTGAAGTACGGTCGCGTTGTC
TGGTCATCCTCTTTAGAGCAAGAAGGGACATAGAGGACACGCACAGGCTCCTAACCTCATTTGCATGTGAGTGAACGCC
TTCTCATTGTCCAGAATCCATCAGGTAGCTCTATGTCCTTGGGAAGGGGCACTGTCTCTTCCCCAGTACACTCTTGTGA
GAGAGGCAGAGGAACACCAGAAGCCTGTTCAAATGCAGAGATCTCTACTTACCCATCCTACCTCTCCGATGATACCAGA
GAATTAGGACAGCATCATCTTTCATTTTCCTGGAGCTGGCACTTTCTTTCCTTCCTTCCTTCCTTCCTTCCTTCCTTTC
TTTCTTTCTTTCTTTCTTTCTTTCTTTCTTTCTTTCTTTCTTTCTTTCTTTCTTTCTTAGTGTAGTAACTATGCAGAGG
GAACTTTCAAGGCTCAGAAAGGTTAAGAGAACCAAGGGCTGTGCTGGTATCTCAGTAACTCAGTTGAGCCACACCACAG
TTCCTCTGAGGGGATGTTCTAACTGTTCTAACTTCATTCCTGTTGCTGTGATAAAAATGCCCCGACACACACCTTTACT
TCTAGAACACAAGAGGTAGAAGCAAGTGGATCTCTGTGAGACCAAGGCAAGCCTTGTCTATATAGCAAGTTTCAGGACA
GCTAGGGCTATCTCAGAAAAACAAAAGTCCTAGATAGAAAGGCAACTCAGGAGAGAAAGGGTTTTTTTGTTTGTTTGTT
TGTTTGTTTTTAGTTTATAGTTATTCCAGGTTACAGTCCATCATTTTGGAGAAGTCAAAGCAGGAACTTCAAACAGCTA
GTTGCATTATGTCCACAGTCAAGAGAATAAATGTATGCATGCTCACTTGGTTGTACTCGGTTGGATTTCTTCACTCTCA
CATAGGTCAGGATCTCTTGCCTAGGGAATGGTGCTGCCCACAGTGGCCTGGGTCTTCCCACATCTATTAACTTAATTAA
GAAAATCTCCTATTGACATGCCCAAAGACTAAATCCACTGTAAACTTTGTTTTGTTTTTGAGGCAGGGTCTTACTATGT
CCTGGAACTCACTCTGTAGGCTGGCTTCAAAGTCACAGAGATCCACCTGCCTCTGCCTCCCAAATGCTGGAATTAAAGG
CATGCTCTTCCATGCCAGGCCTATAAACAATCTGTCATTGAGATTCACCTCTCAGGTGATTCCAGGTTGTGTTGAATTG
ACAATTATAAGTAACTGTCACAGGAAAGATACCCAACTGTTCCTTCAGGTCTCTTCAGCGACATTGGCGGGAAACACTC
ACTGCAAACCCTGCACAACTCACACAAGGTTGACTTAGAAGCACTATAAAGCTCAGTGTAGGAAACAGAAATTGTCTCA
CCACGTGGGAGGTGGGACAAAGTGAAGGAGATCCGAAGCATAGAGATCAGGAAAAAATCAATCTTATCACACTCCTCTC
TACTTGCCCAGAGAGCAGAGATGCTAAGCTGACCAGATTGGCAGCCAGCAAGGGCATCCTGACTTTGGGTCCTGGCTCT
GTCACCTGCTTGCCAATCAACCTCATGTGAGTAGCGTCCTTACCGAATGACTTCCCAAGCCCAAAAATGGTCTTTACAG
CATCTGCTTTCTGGGGGTTGGGTGGACATGCTGCAAGCTCACATGGAACTCAGCACACAGCTGGCCTTCTGTTAAAGGC
AGCTGTTAAATGTTGTTGGGTAGGATAAGAGGAAGCTGGAGAGACTGTGAGATTTAGAAGGAAGAGTGAAAGAAGCCAG
AGGGGGCTTCCCAACATTTCCTTCCCTTTCCTCCCTTTCTTTCTCCATCTTCTCCATCATCATCATCATCGTTATCATC
ATTGTCATACCACCATCACCACCATCGTCATTGTCATCATCATCATCAGTAACAGTAATAACGATATGCTGCACTAATG
CAAATGAAGAAGTTTAGGGGAAAGAAGGAAATAAGAGAACAAATACCCATTTTGCATTATTGGCAACCAAAATATTTTG
TTTATAAATAAGCTTAAATCCTCTAGATACTGTGTCTTGAAACCTTTTCTATGTTGCTATCAAGGAATAAGACAGGTTT
AGTACCAAGCAACCTCTAAGTCAGAGGCTCATTCCTTGTTCATCCAGTGTCCCCTGTAGCGCAGAGTGACCCTCACACA
GGCTGCCCTCCATAAGATGACTTAACAATGGAGACCTCTTAAACCCGTGGCATCTTTGGCTGAGCACAGGTCTTGATGT
AATAGAGAATTCAGAGGGTCTGGAAGGCTTGAGTTCAGAATAAATATTTATCACCTCCCACCTCCTGGTGATGGTCAGA
ACTAGTCCCTGAGGCAAGACAGAGAGTGGGGAGGGCTGCAAATGCAGCCAGCCGGGGGGCCAGGAAGAAGAAAATTGAG
AGCACCACAGAGTGATGTCTTCAGCTTTAGATTTGCATACCTCCAATGAACTCCATAATTCATGTTTAGACATTTTGAC
TCTTGCTAGTTTTTCAGTATCACAAATATAACTTGGCTCTCCAAGTTTCGCTGGGGTTGTAGTTAGAATCGGCGCTAGC
TTTGACAGTGTGCTATTATCAGATCTGGGAACCACCAGTTTTGGGCAGCTCCTCGGCTCCCATCCCCCATTGCCCCAAA
TCATACTGTTTGCATGGGCTGGGGGGTCACCATCCAACTTCTCAACCAGAAATCTGCATCAAGAGTTGCACATGAGGCT
CTGGGCCTGACACCCTTGTGGGTATTGGTCAGCACATGGGACAGATATAGTCCATCCATCCATCCTGCAGCCTCTACCC
ACACCACCTCTACTGGAGGGTGCTTCACAGAGTGATGCTGAGAATTCTGCACACACAGTATGCTGGTCTGCGGCACCAT
GCAGAGGTACAACAGTACTGAGTCCTGCCTGGCTACGGAAGGGATAGCATGACAAGTGGGATAGAGCCAGCAGGGTGTG
GACCTCCACAAGTGTTGACAGTTGCTGGGTGTGAGGCTTGTTATATATATGTATATAAAACATCATCTTCCTCTTTCAG
GAAATATTCTCCTTGCCACGGTTATTGGGGAATGTCCTCTCTGCCAAGTTAATGATTCCGTGATAATTAGAGACAGCAT
AGTCTGGGAGGCAAGGGTGAGTCTAATGTCTCAGCAAAATGGTAAACACTGGGACCTTCAGGATATCCCTTAAGGCTGT
GTGAAAGAACCCTGAAAACATGAATTCAAAAATATATAATTTCTCAACTATGTAAAATATAAGGATGCAATATGAACTA
TATGAGGGGCTTCACAGACCTAAAAGAGCAAAGGCAGCTGCACTATGAGCCAGCTTGTCAGAAAACTATTAAGGAAAGA
GACAGAGAGATTTGGGGAGTGGTGATCACAGGGCAAGATCCTAGTTAAGTTTATTGTTTGTGCTTTAAAAAGCAGGCAG
ATTTATGAGTTCAAGATCAACCTGGAAGAGAGCTAGTTTGGACCCAGGTGCGGTGAGAATCGTAATCTCAGGACAGGAT
CTCACCCAGCTACTTTATTGTCTGTGCTTACAGAGGCAGGCAAATTTCCGTATACATTTGCGATGTTAAAAACAAATGC
TGGACTGGAGAGATGGCTCAGCGGTTAAGAGCACTGACTGCTCTTCCAAAGGCCCTGAGTTCAAATCCCAGCAACCAGG
GCTCACAACCATCTGTAATGAGATCTGACTCCCTATTCTGGAGTGTCTGAAGACAGCTACAGTGTAATTACATATAATA
AATAAATAAATATTTTTAAAAATGCTGTCTCTTTCTAAGAATCAAGGGGCTGGGGTCATGGAATACTGATTCATGAGAT
AATCAAAAGGGAACCTGGAGTAAATGACTGAATTAATATGTAATTAAGACTGGGCTATGGTCTGGGAGAGCTGAACTAT
CTGGATGAGCTGTCTGGAGGTCTGTAAAAAAAAAAAAGGCTGTTTTGAGCAGAACACAGGTTGTCAGCTTGTACCCCAC
AATTGACTTCGACCAGTTCTTTAGCTACTCTCAAATACCCCTTCCTCAGAACCCCTCTCTGAGCTGAGGCTGGTCCTTG
GCACAACAGTGTGCCTTGGCTGGCTCTGTACAAACTTGGCTGCGTGGGGCCTGAAGCTGCATTGTTTGGACATGGGCCC
TCACACAGTCCGTCATCAACTCTACTCCTGTAGCTTCTCCCAAAAATGCTCAACGCAAGTTGAGTCACCTATTCTTAGG
GGGAGTTTGACTCTGATGACTCAAACATTGTCATCTCCCAAGGCCCTCACTTAGAGAGAGCTCAGCTCTCTTCATTTTT
ATGTGTCCTATCTGGTTGAATCTAGGAGCTCAATAAATAAATATAAAAGGGGGTTAAGGACAGAAAAGAGTGAACATGT
TATTAACCCCACATGCACATGGGAATATTCACCAGAGTGTGAATTGCAAAAGAAAAAGGTGTGGATTTTATGCTTTTTA
GACAAAGAATATTAGGGTTGTGAAGGAATGGCCGGGCAACGGAGATCTGGATGAGGTAATAGCTTCTCAAGACATCACT
AAGATGAATGGGGGCAATGATGAACCTGCTAGAAGATAAAGTTAATTCTACATTTGTGTGTTCAGGATCACTGCAGCCT
TACCAGCCTCCAGGACCAGGGCCATTTCCTAGCCTTGTAGACTGTGTAATCTCGATGGGCAGCTTGGTAGTATTTGGAG
TCACCTAGCAGACACATCTCAGGGTGTGAAGGACATGCCTGGAAAGGCTTTCCAGAGAGGATTGACTGAGGGGGAGGGC
TCATGTCCATGCAGGCAGCACCTTCCTCCCAGTGGGTAGTCCAGATATAGAGGGGTCTGGTGAAACATCAGGGTCACCT
GCCTGCCTTTGCTCCCCTCCACCACTGCCGTCACACATCAGTTTCTTTGGCCTCCCAATGTGGACTGAAGGCCAACTGC
TCTTCCAGGTGATCCCAGGCCTTTAACACTACCTAGGATCAATGGAAGCGTCTCTGCGGACTGAGCAGTTATCATGTTC
TTTGTCCGTTTAGCGAGTTGATAGGCATTATGGGACTATCCAGTCCCTATGATGCATACCAATCTAATAAATGCTTTTT
TTGTGATATACAGTCATTCTGTTGGCTCTGACTCTATAGAAAACCATGAATGATACAGTATGTGAAACACACTCCTCCC
CAAAAGATTTTTTTTAATTATTATTTAAAATAAATTAGTTTACTTCACGCATATGGGTGTTTTGCCTGCAGATATGTCT
GGTACCACATGTGTACCTGGTGCCTGTACAAGTCCAAAGAGGGTGTTAAATCCCATAGAACTGGAGTTATAAACAGTTG
TAAGCCGTCATGTGGGTGCTGGCAATTTGAACCTGGATCCTCTATAAGAACAACAAAATGGGGTTGGTGAGATGGCTCA
GTGGGTAAGAGCACCCGACTGCTCTTCCGAAGGTTCAGAGTTCAAATCCCAGCAACCACATGGTGGCTCACAACCATCT
GTAACGAGATCTGACTCCCTCTTCTGGAGGGTCTGAAGACAGCTACAGTGTACTTACATATAATCAATAAATAAATCTT
TAAAAAAAAAAAGAACAACAAAATGATCTTAATAATCCAGCCATCTTCCCAGCCCCTCCCCCAAAACACACACCATTTT
TTCTTTATTTTGAGACATGGTCTCACTATGTATATCTTCAGTTGGCCTGGAGCTCAGTATGAGACCTTGTTGGCCTCAA
AATCATAGAGATCAGCCAGCCTCTGCTTCTGGAGTGCTGGGATTGAAGCTGTGCACCCTGCAGCCAGCCCAAATGGCCA
TTTCTGAGGTTATAGTTCTTGAAACCATTTTTGCTTTAATCGACATTCTAACTCGGTTCATTTTGTGATGGGTTCTTCT
TTCTTAGTCGCTTTATCCTGGTGGCAGTTACTTTCATCACCGCTATTGACCAAATGCCTGGTGGATGCCTGTAACAGGA
GGAAGGTTGATTCGAGGTTATGGCTTAATAAGCTTCAGTGTGTCACAGTGGAAAGGGCATGGCAGAGCACAGACCTCAA
CGGCCAGGAAGCAGAGAGAGGATGCCAGCTCCCTTCTTCTTTCTCTTAGTCTGTCCAGGCTCCCACACACATTCAGAAC
AGGTCTTCCCACTAGCAAATGGAAACAAAGTCTCAGCCTACAAGTGTGCTTCTAATCTCATGAGACCTTCTCAATGCCT
TCACCTTGCCAGTTAAGATTAAAGATCACAAACTTCAGTGTTGCCCTTCCTAGGTAGGTCTTATTCATTCTTCACCCCA
AATCCCTAAGCAACCCCGAATATTAGTAGATATAAACTCCTACTTGCATAGACAGCCATCCTCTAACACAGCGGTTCTC
AACCCGTGGCTGTGTGTGATCCCTTTGCGCAACCTTATTTCCAAAAATACAATTATTACATTACAATTCATAACGGTAG
AAAAATTAGTTATGAAGCAGCAACGAAAATAATGTTATGGTTGGGGGTCACCACCACATGAGGAATTGCATTAAAGGGT
CACAAGATTGGGAAGGTTGAGAAGCCCTGCCCTAAAATCACTCACTGGGAGCACTGGCCATCCAGAACTTTCTGGACAT
CGCTAATGTTAACCACCATCCAGAAGAATGTCAGACTTCACTTAGACTATTTTTTGGGGTAAATTCCCAGAAGTAGGGT
ATCACGGTCCAAGCATAACAATATTGTTCATGGTTCTTACACCTAATCCTCACAGTATTCAGGAGTAGCGATTGTTTTA
ACTTCTGCCATTGTCCTGGATGGTGACGTTTCTCCTTGTTTTGATTCATGTTTCTGAGGTGCCTGCATCATCCTTCCCG
AGCTTGTCTCCATGCAGGGGATTTGTGAATGCAGATGTTGGGTAACTTCTTCAGGCCTTTCTCTCTTTCTTTCTTTCTT
TCTTTCTTTCTTTCTTTCTTTCTTTCTTTCTTTCTTTCTTTCTTTCTTTCCTTCTTTCTTTCTTTCTTTCCTTCTTTCT
TTCCTTCCTTCTTTCTTTCTTTCTTTCTTTCTTTCTTTCCTTCCTTCCTTTCTTCCTTCTTCCTTCTTTCCTTCCTTCC
TTCCTTTCCTTCCTTCCTTCCTTCCTTCCTTCCTTTCCTTCCTTCCTTCCTTCCTTCTTTCTTTCTTTCTTTCTTTCTT
TCTTTCTTTCTTTCTCTTTCTTTCTTTCTTTCTTTCTTTCTTTCTTTCTTTCTTTTCCTTCCTTCCTTCCTTCCTTTCT
TTCTTTCTTTCTTTCTTTCTTTCTTCTTCAGGCTCTCTCTCTCTCTCTCTCTCTCTCTCTCTCTCAAGGTTTATTCATT
TATTTTATGTATGTGAGTACACTGTCACTCTTTTCAGACACACCAGAAGAGGGCATCGGATTCCATGTGAGCCACCATG
TGGATGTTGAGAATTGAACTCAGGACCTCTGCGAGAGCAGTCAGTGCTCTTAACTGCTGAGCCATCTCTCCAGCTCCAG
GCCTTAATTTCTTAAACTGATTTTGGGAAGCTCTTTGTATAACAAAGAAATCAGGCTTCTGTTTGTGGTGATTGCATCT
TTGAGCTTCTGTGCAACCATACACATTCTGGTCTCTTTGTGTTTTAATTTTGGTTACTTAATTATATTGTTTTGTTTTA
TTTTCATTTTTATTTTTGTAATACAGAAGTTTTAAATTTTCATTTACACAAATCTGTCAGTCTTTTGTGGCTTTTCTCT
CTCTCTCTTCTTTGTTTTTACAACAGAGCTTACAAATATATTTCCTTTGCAAAAAGTATTAATTAGTATTTGATTCTGT
TTTCCTCTACATTGTCACATATGTGTTATATATCATTATATATTATATTAAATATATGTAGATGTAATATATAAATATA
TTTGGACTAAATGAAACATTTTAGAGTCATGCCATTTTATAATTTGTTATTATTATTATTATTATTATTATTATTGAGA
AAGAGTCTCATTGGCCTGAAATTCACTCTGTTGATCAGGCTGGCCTCAAACTCATAGAGAGCTGCCTGCCTGGTGCTGA
AATTAAAGGCACTTGTCACTATGCCCAGCCTGATTTTTTTTTTTAATTATTTTACTATTGTATTTTATATTTATGGTTA
TTTTGCCTGAATGTGTTTGTGTACCACTTTCGTTCCTGGTACCTATAGAGGCCAGAAGTGGGCATCAAATCTGCTGGGA
CTGAAGTTACAGACGGTTGTGAACCACCATGGTGGTGGGGGGAGGGCTAGCAATCAAACCTGAGTCCTCTGGAAGAGCC
TGTGCTCTTAATCTCTGATCCATCTCTCTGGTCTCATACTTTTTTTTTTTTAAAGTAAGAATCAACTCTTGTCTGTAAG
TGTATTGAAATGTTATTAATATTGTCAGGGATTGGTTCTTGACCATGGGATGGGTCTCACTTTGGGTCAGTCATTGGTT
GGCCATTCCCTCTGTCTCTGCTCCATCTTTGTTCCTGCACATCTTTGGATTTTTTGAGTAGGTTGCTGTCCTTATTCCT
CCACTAGGAGTATTGCCTGGCTATAGGAGATGGCCACTTCAGATTCCATATCCTCCATGTCTAGAAGTCTCAGCTGGAG
TCACCCCATAGACACTATGGGACATCCCTCTCCCCACGCCTTTACGCCCATATCAGGTCTCTGGCACATCCTAGAGATG
CCCTCTTGCCTGACAATTTCCATTCTTTCCTACTCCCTACACCTGATCCCCCCTTCCTCTCCCCATTCTCTCTCCCTCC
CAGTTCTCTCCCTCCGTTCACACCTGATATCTATTTTATTTCCCCTTCTGAGAAAGATTCAAGCATCCTTCCTTGGGCC
CTCCTTGTTATTTGGCTTCTTTGGGCATGACTGTCCTCTGAGAGGCTCCACTCAGCAGACGATGAGAACAGATGCAGAG
ACCCACAGCCAAACATTAGGTGGAGCATAGGGAGTGTTTTGGAAGAGTTGGGGGAAGGATTGACGGACTGGAAAAGGAT
AGGGGGGCCACAAGAAGACCAATAGAGTCAACTGACCTGGACTCTTTGGGGTTCTCAGAGACTGAACCATCAACTGAAA
AGCTAGCATGGGCTAGATCTAGGCTGCCTGCACACAGGTAGCAGATGTGCAGCTTGGTCTTCATGCAGGACTCCCAACA
ACTGGAGGGAGGACTGTGCCTGACTCTGTTGCCTGCCTGTGTGTTGTGGTGAGTTTTTGGTTGCACTACTTTGATGTGC
CTGGGTAGGCCATGCTATCAGTCACCGGCCCAGATTCTCTTGGATCCGAGGATGAGACACACACACACACACACACACA
CACACACACACACACACACACTAGCTCATTTTAACCTGCCTTTCTGGCTCAGTGGCTGGGTTCTTTTGGTTTTTCCAGA
CAGGGTTTCTCTGTGTAGCCCTAGCTGTCCTGGAACTCACTCTGTAGACCAGGCTGGCCTCCACCTCAGAAATTCGCCT
GCCTCTGCCTCCCAAGTGCTGGGATTAAAGGCGTGTGCCACCACTGCCCTGCTGGCTGGGTTCTTCTAAGCCTCTTGTG
GCTAGCTTGGCCTTCCCTTTACTCCTAGATCAACACTTCTAAATCTTCCCTCAGTTTGTTGCCCGGTTACCTTCTGTGA
AGTTCATTGGTCTTGATGCCCAAGACGCTTTCAGGCTGCCCTTCTGTGGGCTGCTTTCCCTTTCTGCGTACATTTTGGA
AGACCTCCACCAGCATCTTCGAGTCTGTTTCTTCTCTCTCCCCCAAGTATCTTGAGTTTCCTCTTCCTGTCCTCTCCTC
TCTCCTAGCCTGTGGGAACCTGGAAGTCCTGCCTCTTCCTTCCCTCCCAACCACTGGCTGCTAGCGTCTTTATTGATGG
ATCAAGAACTAATTGGGAACAAGGACCTTAGTGTATGGCCACGCAGACTCCCCGATCAAAGCATCAGAAACTTTTCCCT
ACACCCATGGAACCCGCTTTCCTAACTGGGCCACCTTGTCTGGCATCAGTGGGAGATTGTGTGCCTAGTCTTGCAGTGA
CTTGATGTGCCAGGGTGGGGTGATAATGGGGGGGGGGCTCATCAGAGGTGAAGGGTAGGGGGGAGGGGGAGGGAAGAAG
TATGTGAGGGGAGACCCGGAGGAGGGGGGGCTGTGATTAAGATATAAAGTGAATAAATAAATAAATTAATGAAAACAAT
CAAGTCTTAAGGCAGAGGCTATGTAGAGAATTGAGGGGAGAAGCCATCTACACAAGAGGGTGCTATGACAAACCTAACT
CCCCTTTGCCACGTAAAAATAGTATATTCACATAATGTGAGGGAGCCATGATGCAATCTATCATAGGGCTCAAAATCCA
GTAAGGCTGGTGGGAGTTAGGTGTGAAAGAGAGTAATGGTTTCCTCCTTCAACATCTGAAGGTGAGGTAGAAGAACAAC
CAGGCTGGGCCCTAAGCCCAACCCCATCATGTGGCTAAGGAGCTAGGGCAAGCCTTTCGCCATGTTCAGAGCTGTGTAA
ACTTTCCCCCACTATTAGCTCAGCTCCAGTTCTTGGCTTTAGGCTCCACACTAAGGAGTAATGGTGGGTGATGAACAGA
TAATTCCTTCATCTCTAATTCAGAAACATTGGCTTTCCCTGACCTCTGCAACCTCCTGACTCACAGGCACTGCGCCATC
TAGACGTTGGTGAACATCTCAGCAGTGGAGTGACTCAGAATATCATAAAGTTCTCCTCATTCGTGGAGGGAGGACAGCA
CCGCTTCTAGATGAGAGACTGCAGAATACTCACCTCCTTTGCTCTGAGAGTCAACTCCAGCTTCTGACATTTATAAGGC
AGGATGAGATAGTCTCCAAAGATGGCTGCCGGCAATCCCCTATCTCTCTAAATGCACTTGCATAGAAAAGAGGTATCTC
ACCAGGTTAGCAGAAGTGTATCGACTAATGAAGTGTAGAGTAAGTTCTGGGTTTCTTGAAGCGAAGTCACAATAGGCTC
TGCATGGTCCTCACTGCAGTCTTTCTGGACCCCTGTACAGTTCTGTGAGGAGCCCAGGATATGGGGATCCAGCTGAGAT
TAGATGAACTTCCAGTCAATAACAGGATAAACTATGAACCATCTCGCATCACCTGTGGAACAGCTCTGAGACTGGCTTT
TCATGGTCACTCGGGGACCCTGATCCTCTAATCATTACTCTGCTATTCAGATGTGACCACCACATTGTCACCATCAGGT
TTAGGCTTCTAGACAATATGGAAAAAGGAATCAAGGAGGGAGGGGGGAAGGAGGAGGAGGAGGAAGAGGAGGAGGAAGA
AGAGGATGAGTCATCACCAATGCCTGAAAGTGGCTTCATCAGTTCCCTTCAGTCCAGTATCCTTGCCAAGACCTTGATC
CCATGACCTCCCCTAAGCCCAAAGGAAAATGGAAAATGAAGTCCAGGAGGCAATGGACAAAACAGGACATTGGAAGGGC
AGTGGCACAAGCTGGGTCACAAGTTCTTATCTTTTTCTCAGTCTGGGAGCTGAGTCTAGGCAAGAGCCACATGTTCGTC
TTGGTCCCTAGTGATGAGCCAGTGATAACCCAGGGTGAAACTCCACTCCTATTTTCAGAGTAAATGAGGCCACAGAAGT
GGTCTAGGCTGGTACAGTAAAGTTGTGTGCAAATGCAGAGAGAGAATCTCTCTCCTGAGCCCGGAGAGAGAAGACAGAT
GTGGAGGAATTTAATTGGGAGAACACAGGGCTTGATGGATCTTGCCAGAACACAGAGCTGTTAATGTTATGATTTGGGG
GGGATTTAACTTGACAGCTCCTTGGCACAAGTTTAAAGAAGTGGAGCTGAATACCTCACTTTGCAGCAATTTATTGAAC
AGATTCCAAAATTAAGAAATTCCTCAACTGTTTCAAAATCATGTTAAGAAAATGCACGCCTGGAGCTAAAAGGCACTCT
GGTTCACTGCCACCTCTGCTACTATCAACAAGCCGAAGCACCCAAGTGCACATCAGAGCCTCACCTGGCTGCCATTCCA
CATCCCCGTCGGGCCTTCGTGGCTCCAGCAGACCCAGAACACAGATGCACACACTCACGTGCAGATGCCAGACACCAGT
TGTCTCCAGGAAGCCGTGCTTTGTGGCTGAGCCCGGGCTGAACATACCACTCCTGAAAGTCAATACAGGCTTTGGAATC
TGTGCAGACGGAGTCTGAGTTCGGGCTCAGCTCCTTGCTACCAATTTTGGTCAAGTTATGTCACCTGCTCAACTCTGCT
TCCTTCACCTGCAAAGTCAGGCTTTATTGGGGCTACATTACTGGGTGTGTAAAAACTGAATGAACTTCTGGGCATTGGT
CCTACCAACTAACAATTAACAGTAAACAACAATTCAAAGAATTATGAAAGCTGGGGTTTCAGGCCAGTGTGGTACTTCA
AGTTCAAGGATATTAGTAAATATCCCAAAGCCAGGAGTAGTGGCTCATGCTTATAATCTCAGTGCTTGGAGGGCAGAGG
CAGGAGAATTATCATGGGGTTGAGGCCAGCCTAGGCTAAATAGTAAAGTTCTAGACCAGAACAGAATGAGACCGTATCT
TGAATGAATGAATAGCTGACTGAATGAATACACAAATAAGTAAATAAATACATCAGAAATGATACCTAGTTATAGCTAC
TAATAAAACAGTTGTGCATACATGGAATGTTATTCATTCCTATGACTCCAGCCTGACCTCTGCTGTGATCAATCTCACT
CCATGATTCCTAGGTGAAATGTGGTGTTGCTATGTTCAGTACTCCCACTCCACGTCCTCTGTACTCCTCCTGGGTCCAG
GTGTGATCTGAGGGCTTTTCCATCAGACACCTTAAGGAGAGAAAAGTAACTCCTTTTGGTGTTTAGGTTACCCATGGTT
TGGGAGCCATGCTGTTGTTGGCTGCTGACCTTCTGTGGCCTATAGTAATGTGATCTCTGGTCAGTGGCATATTCCGTCC
CATCCTACAGATCCTCTCCTGCCCTTCCCTCAGTCACAGCAGCCAGGCCTGTCCCTTCTTTTTTCTAGGGCTGGCCAAG
CTCATCCTTCCAGTGGATGGGTATCACCCTGTAGGTCACCGTAGCTCCTACAGTCAGCTGACTGTGAGCTCACAGAAGC
TCTACAAGCCACATTTGGGGGTATGAAAAACTGGGTTCACTTAAGTCTAAAGGCCTCAAGCCACCATTCTTTGGGCTTC
TCTTTACCAGAGGGTAAAGGCCTGGGTGAGCAAGCACAACTGGGAAGCCAAGCGCCTGCTCTTCCTTCAGTCACAAAGC
CAGGCTCTCCCAGGGATGGGGCCTTCTCACCTCATAGCAGAGCTCACAGTGAAAAGAAGGGATATTTGACTATTTACCC
TCTGTCTCTGTCTCTGTCCCTGTCCCTGTCCCTGTCCCTGTCCCTGTCCCTGTCCCTGTCTCTGTCTCTCTCTGTGTCT
GTATCTGTGTATCTCTGTCTCTCTCTTTCTCTCAGCACAAAGCTTGAATACTTTGTATAAATCGCAGGGTGTGCACAAG
CGCGAGTGCTTTTCTGGCTCCTTTATCTCCCAACCAGCAGAGAGTAAAACTTTTTGACATGCTATGCTTTGGCGTTTGC
TCCTCTTGTTATCAACAGCCATCTACGGTGGTTGAGGGGATTACCAGGCTACGTGAGGGCCCAGCTATCAGCTGCCCTC
AATGGATACTTCACACTCTGACTTGTCTCAGTCAGTGCTCTTACTCGCTGAGCTCTAAGTCGCTCCGGATCTCAGTAAG
CTCCTGTTGGAAAGAGTGCCTGGTTACTCTGTCCCCGGAGTGCACAGAACCTCCCCCTGCAAGCTCAGTTCACACTCCA
CATCTCCCTCTGACCTTTCACTGAGTTTATTGTCATCAAAGTTATGTAGTTAAAACCAAACAAACAAACAACAAGACTA
GCAGGGCAAACAGTGCCCCTCTGCCTTTCTTGGAACTTCCCCAACCCATTTCGTTCCCCCAGCTGACTGCTGTGCTATT
TCCCACTACATTGCCAAAGAATAGGGCTCTATTGCTGCTTCTGGGGTTTTCAATGTAGCCATTATCTGCCAAGTTTATA
GTATGGAAGATGGAGGGTTTGTTGTTTATTTATTTGTTCATTTTGTTTTGTTTTTGAAAAAACTCTGTCACATACTACT
AGCTTCCTCTCTCTACACTACACTTAGAATTTATATTACTGTAACAATATAAACACTAATCATGTGATAATGTCCCATT
TTTATAATTGCCTTCCCTATTTAACAACCCTTTCTTCTTCCTGGTGTTAGATATTGCCCTTTCCATACTTTCCTGTACA
CTTAACTCTAAACCAGTGCTCTCTTCTTGGGACCCTCGTGCTCTCTCTGAGGACTCTGTCTTCTGACTATCGAGCTTTT
TGTTTTCTAGATCCCATGTGTTGGGTAATCTGAATCGGCTTGGCCATTCCACATGGTCTAGATTCTTTCAGGAAAGACG
TATGGCTTCTAGTGTACGTTTCTTCCTATTACTTGGAATAGGAAGTATATATATATGTACACACACACACACACACACA
TATATATATATATATATATATGTATATATATATACACACACACATTACTAATGTATACTAGAGCAAATTCTGCCAGTGT
TCAGGGGTTTTTGTTGTTGTTTGGTTTTGTTTGGTTTTGTTTTTTAACAAACAGGCATAAAGAATTACAGGGACCTTCT
GGGTGATGTCCTTCGTTTCTATCTATGGATATCCTGATGAAAATCTTTTTTTTTTTTTAGTCATTCTAGGGCAGATGCA
ATGTATTTTGACAACAGAACAGCCTACATCGCAGTAGGATTAGGTAGCATGGGGCTGTGACGTACGTGTGTTTGTGTGT
TTGCATGTTTGAGGCACAGGATCAATGAGATGCTAGACCACAAAACCTGTTTTGAAGGTGCGAGGCTTGTGAGCTTGAG
ATTCTTTACCTGTACACGAAGAGATTTTTTTAAAAGATTTATTTATTTATTTTATGTATGTGGTACACTGTAGCTTTAC
AGATGGTTGTGAGTCTTCACGTGGGTGTTGGGAATTGAATTTTTAGGACCTCTGCTCACTCTGGTCAACCCCTCTTGCT
CAGTCCCTGCTTGCTCTGGCCCAAAGATTTATTATTATAGGTAAGTACACTGTAGCTGACTTCAGATACACCAGAAGAG
GGCGTCAGCTCTCATTGCGGGTAATTGTGAGCCACCATGTGGTTGCTGGGATTTGAACTCAGGACCTTCGGAAGAGCAG
TCAGTGCTCTTACTCGCTGAGCCATTTCACCAGCCCATAAGAATAGATTTTAATAGCTAAAAAAATTAGATGCCAACAC
TCTTGGGGTGTAAGTGCTTAATTCACTGGGTGTGTTTGAAATGCCAATTCAGTGTCTCTGTGATATAGACTGACATTAA
TGTATACACAAGTGTATATTAGTGTTGTTACATTTTCACACAATGTAACAGTACACCTTCTTGTTCTTGGTTTATTTCC
TCATTATGATGCAACAAATCCTATTTAAAAAAGAAAAAAAACTTCCTAAGGAATGATTCATGGGAAAGAATAATTTAAC
CCTGTTGTTATCTTATGATGCATTTTGAGATCTTGATTGAAAGTATCTTTATTCGGCTTCCCCCATCAGTGGATAGTTT
GGCTGGGGATGGAATTCCAAGTCAGAAATGGCTGCCTCTTAAAGTATTCAAGGCAGTGCTTCCCGATGATACCACTCCT
CTCTTTGTGGAGCCGTTAGCTCCCACATGTCTGTCTCAAGCTCTCTTTTCTTGCAATGGAGCTATAGTTCTGTTGAATC
CCTTCACCATCTTCTAAGGAGTCTCTTTACCTCTCTCCTGTATAAAAGTCCAAGCTTCCTGAAATCCATGCATCATGTG
ATCTGAATTGGCCCAGCCATTTCACACCTTTAGATTATTTTGGCCAATACCCATGGACTCCACTATTAGTCTCTTCTTA
ATAACACTACTAAAGAATAACTCCACCACCAACCTTTAAGGATCTCTACTGAAGGTCTTAGAATATTATTTGATATATT
GATAAAATACATATTTTCCTAAGGATCTGGCAAAACATCTCAAGCTATAAAATTATGTCCTTGGATTTGGGAAACTTTT
CTTGACAACTCTCATTAACAAAGATCTCCTTTGTCGTCTTTCCTGGAGCCCTTGCTGATTAAGAGGACACAGCTCAGTG
ACACACACCCTAGTGAATCTCTGCTCCCTTCTCTCTGCTTTCTTTGTTTTCATGTCGCTTTCTAGGATATTCCTTCAAT
TTTATGTCCTGACTTTAATATTGCCTTTTTTGTCCCTCCTGCCAGGCTTTGCTATGAACACTCTTTGCGCCTTACAGAC
TTGCCCTTTCATGGGCTTTGTATTATCCCTGGTTTCTCTAGGAACTAGTAACCATATGTTAGGTATTTTTCTACCTTGT
CTTTTATAGACTCCTTTCTGTTTGATTTGCTCTCTGCCTTTTATGTTGAAGAGGTACCTCAGTTGTCCTCAATTTCCCT
TTCATGTTTAGGAGTGTAGTGCTCAAACTCTGATGACCTTTGACTGTTGACCTTTAGCCAGAGTCGTCTGAGTAAGAGA
ACACCATGGGCCAGTATCTTGAGGTATTTTCTCTCAGATCAGACATCCCGGGGAAAAGTTCCCTTTTGGGTTCCGGAGG
ATGAAGGTGAGAAGAGAGCTGGAGGTGCAAGCAGGGGTCTTCCTGTTCAGTATCTCACTTCTCCGGAAGCTCCCATCTC
AGCTGTCACATCTCAGCTGTCATCGGCTCTTGGCTGTCCTTCTGCTTGTGGACTTGTTTGTCCTTCTCAGAACACCATC
TTCAGATCAAGGAGACCAGGTCTCTATGTTTCTCCCTGAGACAACCTAGCCTCCCATTCTTAGTTCTTGAATGCCCCTT
TCAGAGATACCCGCAGGCTCTAACTCCCGAATTTAGCTAAGTCACCCATCTGCCTTCAGCTTTTCTAGATGCAGCTAGT
CACCGCTCTTCTCTGTTCCCGTTTCCCTTTGTCATCTGGGTTTGAGTAAAATCCACTTCCTGCTATTTCATAAAAGTCT
GTGGAGATGGCTGAGATAAGAATCTGTGTTTAATTTGCCTCCATTTTATTTAAAGCACAATTTAACAGTTTTGTTCTTT
CTCTCTTTATTCTGGTGTCGTGTGTGTTTATTGTATGTGTGCACCTATGTGTGCACATTTGGAGGCCAGATGCCAATGC
TAGGGGGACTTTCTTCCATTGTTCCCTACCTTCTGTTTTTAGATGGGGTCCCTCGCTAAACCTGCTCTTGGTGAAGAAG
GGCGCTGGCCCTCAACTGACACTGCAGTTTGATCCCTGGAATCCACACAGTGGAAAGAGAGAACTGACTTCGGCAGTTT
GTCCTGAAACCTTTGCATGTGTGCCATTGTATGGTCATGTGCCTGTGCGTGAGTGTATTTCATGAAGACAGACACAGAC
AGGCAGACAGGCAGACAGACATACACACACAGACAGAGAGAGACAGACAGACAGACACACATACACAAACAGACAGATA
CACAGACACAGAGAGACAGACAGACACACATACACAGACACACACACACACATGCACACACATACACAGACACACACAC
ACATGCACACACATACACAGACACAGACAGACACACACACACACCTGGACACACACATCAAACTATTAAGTAATTCCGG
AGCTTTCCAATTGGCCAGCTGAGCTTCAGGAATACTCCTGCACTCACCTCCCAGTGCTAGGCTTGTGTCCGCTTGGCAT
CTCGGCACCATGCTCTAGACTTGCCTTTTTTTTTTTTTTTTTTTAAGCAAAGGGGTCTCATTCTTTTTGGTCTCCTGAT
CTAATTTGGAAACTCTCCAGACTAGATGGGCCGATGCAGCGTGTTAAATCTCCTCTGTAGCCTTGTTCATTTTCCCTTT
GAGGCTCGGGTTCTGTGCACAATGGTCCAGTCTCATAGACCAACTTCTGCTTTGCCACCAGACTGTTTAACATGGGGCT
TGGTGTCACCTCAGAGGCTTCCCCCCAGTTCCAGCTTCGCTTTCACCTACGAGCAGAGCCTACAGTTCTTTACCAGCTT
CCACCGGCTTCACTGGCAGTTACACACTTGATCTTGCTACTAAGTCCCCGCTGCTTCCTTGCTCTTGGGTTTTAACTCA
GAAATGACTTTGAGTCTTATTCCAGAAAAAAAAAATAGAAGCAACCGAATGTGACTGTCTTCACTCTCTTTCTTACCCC
ATGAAACCATCTATATCTGCAGCCATTCAGGTCTCCCTACCCAGCACACGCTGTCCCGTCTCTCTAATAGTCTCTTTTT
AAGGCTGTCTGGGAGTCCTCGGATGTCTCTCCCACCACAGAGGCCATGGGTGGCTTTCAGGCCCAGGCTCTCTGCATCT
TTGGAAGATTTCCTACTCTCCTCTTGTGCTGTGTGTCTTTGACCTCTTTAGCACATTCCTTTCAACTTAGGAACAGCGT
AAAATTTTCCATCTCTCTCTCTCTCTCTCTCTCTCTCTCTCTCTCTCTCTCTCTCTCTGTGTGTGTGTGTGTGTGTGTG
TGTATGTGTGTGTGTGTGTGTGTGTGTGTGTGTGGTGATAGGGATGGAACCCAGGATGAAACCCTGCCATGTATTCCAC
AAATGCTCTATGGCTGAGCCACATCCCCAGCCCTTGGGTTTTCCCCCATTTTTAAAGAAGCCCTCTAGATCCTTAATGA
GCCCCCTGGTGCTGGTTCAGTCCTTTAATCCCAGCACTCAGGAGGCAGAGGCAGAAGGATCTCTGAGTTAGAGGCCAGC
CTGGTCTAAGGAATGACTTCTAGACAGTCAAGGCTACACAGAGAAAACTTATCTTAAAACAACAACAACAACAAAAACC
AAAAAACCAGACAGACAAACAAAAGTCTTTATTGCCTCCCTCCCCTCCCTCCCTCACACCCTCCCCTCCACTTATTATT
ATTATATTTTGAATTATCTTCTGCGTTCACTTTGTCTGCTTCCTTGTCCCCTTTTCTGTCGTGACCTTTGCTTGACATA
GTCTTCCTCAACACCTCGATGTGTATTGGTGTGTACTCGGGCAGCCTTGCTTCCTCCTGGAAGTCTTCACACAGTTGGC
CACTACCCCTTGTGCAGACCCTACAACCCCATCCTGCTTTGCCTATGTCTTCAGCCACTCCTTTTCTATCCTTTCCCTT
TCTTTCCTTGAATATCAATACCCACAGTGCTCCATCTCAGCGAACATCTACCCTTAGTGCTCCATCTCAGTCATGTACC
CACAGTGCTCCATCTCAGCCATCCACCCACAGTGCTCCATCTCAGCCATCCACCCACAGTGCTCCATCTCAGTCATCCA
CTCACAGTGCTCCATCTCAGCCATCCACCCACAGTGTTCCATCTCAGTCATCCACTCACAGTGCTCCATCTCAGTCATC
CACTCACAGTGCTCCATCTCAGCCATCCACCCACAGTGTTCCATCTCAGTCATCCATTCACAGTGCTCCATCTCAGCCA
TCCACCCACAGTGCTCCATCTCAGCCATCCATTCACAGTGCTCCATCTCAGTCATCCACTCACAGTGCTCCATCTCAGC
CATCTACACACAGTGCTCCATCTCAGCCATCCACCCACAGTGTTCCATCTCAGTCATCCATTCACAGTGCTCCATCTCA
GCCATCCATTCACAGTGCCCCATCTCAGTCATCCACTCACAGTGCTCCATCTCAGCCATCTTTTCCTTTTGCTCTGCAT
AAAGCTTTCCAAACAACATTCTACAGGAAACTGAAGCTCAGTGTATTCCAAACTGGAGTCATAACTTCAGCACTTAAAA
CTGTAATGATGTGCTTTTGTTATCTCTGAAAACTTGCGAGAGCTACTGTATTCCTATCTGAAAAATTAGATGAAAATAT
ATTCCTTATAGAATGAAAGGGCACTAGTATATCTCATGACGTATGCTGCTATGGCTCGAAGGTGTCCCCAAAAAGTGCT
GGGAATTTAATTCCCCATGTGACAGTCTTAGGAAGTGGTGCCTGATGGAAGATTATGACACAGGGGCACTACCCTCATG
GATGGATTAATTTCTTTATTACAGGAATGAGTTCATTCCATATGAAAGGATGTTTGGCTCTCTCTCCCTCTCCCTTTCC
CTCTCACTTCTCTCTCCTCCCCCACCCTGTCTTCCATAGGATATAACAACAAGCCCCCTCTACATGACTCTTGCTTCTT
GAACTTCCTTCCCATCCTCCAGAACCATGAGTAAGTCTGTTTCTATTCATTATAAATTTCCCAGTTCAAACTAAAATAG
TTTTGGATGGATGAGGTGGGGTGGGGAGGACTTGGAGTGGAGGATCAATCAGGCAGAGAGGGGGAGTGGCTGTCTTCCA
TAGGATATAACAACAAGCCCCCTCTACATGACTCTTGCTTCTTGAACTTCCTTCCCATCCTCCAGAACCATGAGTAAGT
CTGTTTCTATTCATTATAAATTTCCCAGTTCAAACTAAAATAGTTTTGGATGGATGAGGTGGGGTGGGGAGGACTTGGA
GTGGAGGATCAATCAGGCAGAGAGGGGGAGTGGCTGGGGGCTGGGAGAGGGAATACAAGAAGAGACAGATAAAATTAAG
TCCATTTAAGAGGTAGTATGGAAGTCTAATATAATAGAAACTTCCTAAAATATATACATATATGAAGGTGATCCAAATG
AAATCGACAAATAATGGGGGAGTCAGAGTTCCAGCTGAATGTACTGTCACCAAATGAAGCTTGTTACATCTAATTGAGT
AATTGACCAAAGGGGTCCCATGGGAAGCCCCAAACAGCCCAGGCTGTTGCGAAGGGAATAGGTTCCTCTCCACAAATTG
ATAGTAAGCTCCTATTGCTGAAGATGATCTGTACAACTCACGGAACCCAGAGAAGCTGAGCTGGTGCCTGACTAGGTCC
TTTCTCCCGATGGGCTAGTGTTCTTGGTGTTGAGAGGTACTCTGCATGCTACCAAGGGAGAAATGTAAACAGCAACCCG
GTTATAAACCCTTTGATCTACAAAGCTGTCATGCCAGCAAGATATGACAAAAAGCTTGTGAAAGTAACCCATCAATATC
TGATTTAACATAAGACCCAGTTCATGAGACGAAGCCCATATTCGATGCTGCTTGGATGACCGAGAACCTGAAACTAGAT
AGCCCAGGGACCCAAAGTAAAACCAAATAGTACTAAAGGAATGTAACAACAAAATGACTCCCAATGACATTCTGCAATA
TGCATAGACAGTGTCTTTCCTGACTGTCATCAGAGAGGCTTCCTCCAGCAGCACATGGGAGCAAATACAAAGACCCACA
GGCAGACAATATGCAAAGAGTCAAATCCCTCCTCTCAGGGCTCAGAGGACTCTAAGGAAGAGGTGGAAAGAGTGCAAGA
GCCAGAGGGGATGGAGGACACCGAGGAAACAATGCCTTCTAAAAACAGCAGATCAAGGCACACATGAGCTCAGAGACTG
AAGCGGCATGTGCAGGGCCTGCACAGATATGCACCAGTTAGTGTCTTAGAGTTGAAAGAAGTGGACACCTGCCTCTATC
CCTAACCCAGAAGCTATCTCTAATTGTTAACCACTTAAAAATAAAACTTTAGTTTCCTCCCAAGGAGTCTTACTAGGGT
AACACAGTACCATTAAGGCTACAAGCCCAGCATGGCCAAAAGAAAACAAACTCCACAGTGTCTTTGGAGGTTCTTTGTC
TCATAATGTCCTGTCGGGGGCTTGCTTCCTTCCTTCCTTCCTTCCTTCCTTCCTTCCTTCCTTCCTTCCTTTCTTTCTT
TCTTTCTTTCTTTCTTTCTTTCTTTCTTTCGTTCTTTCGTTCTTTCATTCTTTCTCTCTCTCTCTCTCTTCCTCCCTAC
CTCTCTCTCTCTCTTTCTTTCTTTCTTTTCTCTTTCTTTCTCTCTCTTTCTTTTTCAGATTTATTTATTATATGTAAGT
ACACTGTAGCTGTCTTCAGACACCCCAGAAGAGGGTATCCAATCTCATTAAAGATGAGTGTGAGCCACCATGTGATTAC
TGGGAATTGAACTCAGGATCTGCAGAAGAGCAGTCAGTGCTCTTAACTGCTGAGCTCTCTACAGTTCTCTTCTCTTCTC
TTCTCTTCTCTTCTCTTCTCTTCTCTTCTCTTCTCTTCTCTTCTCTCCTTCCTCATTTTTTTCTTTTTGTTTTTTGATA
CAGGATTTCTCTATGTAGCTTTGGCACTACCACTGCTCAGCTCATATCTTATTTTTCTTTCTTTTTTTTTTCTTTTTTT
GATTTTTCGAGACAGGGGTTCTCTGCATAGTCCTGGCTGTCCTGGAACTCACTTTGTAGACCAGGCTGGCCTCGAACTC
AGAAATCTGCCTGCCTCTGCCTCCTGAGTGTTGGTGTTAAAGGCGTGCGCCACCATGCCCGGCTCTATTTTTATTTTTC
TATTCCCCCCCCCCCTTTTTTTAACCACAAAGGTCTTTTTCACACACACACACACACACACACACACACACATACGCAT
ATGCATATGCATATATATGGTTTTTCTGTTTTTTTTTAAATGGGATTCCTGAGTGTGTGATTGAGTGGGTCTCTGAGTC
TATATTTGTTTCTTGAGCCTTTTCTTGACTCTTTTTCTTTTTTTTTAAAAGATTTATTTATTTATTATTATATGTAAGT
ACACTGTAGCTGTCTTCAGACACTCCAGAAGAGGGAGTCAGATCTCGTTACGGATAGTTGTGAGCCACCATGTGGTTGC
TGGGATTTGAACTCTGGACCTTCAGAAGAGCAGTCGGGTGCTCTTACCCACTGAGCCATCTCTCCAACCCCTTGACTCT
TTTTCTTTCTTCTGTTTGCTTTTGTCCTAGTCTAATGTATTTGTTTTATATTATTAGATTTTATTTTATTTTATTTTAT
TAATCCTTTGGAACCTGTTTGTTTTCTAATGAGACAGAAAGTAAGTAGATTTGAATGAGAGGGGAGGTGGGTAGGAACT
GGGAGAGAAACCATAATCAGGGTATATTAAATTTAAAAAAAATTCAAAAAAAGGCAAATAAATAAAGAAATAAGATAGG
ATATTTCCCAGCCTGTGTACTATTCTAACAATGTAGCATGGACCAAGGCCCACACCAAGGACTTATCAAGTGTCTGACA
TACTGTCTGTGCTCAATAGGGGGTCCAACTATTATTAGTAGTAAGAGTTGCAGAATGTGGTGTTTTGAATATGCTTGGC
CCATGGGAAATGATACTATTATGAGGTGTGGCCTTGTTGAAGGAAGTGTGTCACTGTGTAGGTGGGCTTTGAGGGATCC
TAGGCTCAAACTCCAGTGTGGAAGACACTCTCCTCCCGGCCGCCTGCAGATGACAGTCTCCTTTTGGCTGCCTTCAGAT
CAAAATGCAGAATTCTCAGCAACTTTTCCAGACACATGCCTGCCTGTTCACTGCCCTGTATCCCTCTGTGATGATAATG
AACGGAACCTCTGAAACTGTAAACTAGTCGTAATGAGATGTTTGTCCTTTAGGAGTTGCCTTGGTCATGGTATCTCTTC
ACAACAATGGAACCCTAACTAAGACACAGAGGGAAGACGCTGATATTAATATGACCATTATTCTTTTCACTAGCCTGGC
ACCATGCCAGCTATCCCCAGTTTACTACTTAGGATTCATCCTTATCCTCTCTGCCTATCAGTGTTTATCACCTACATGC
TCAATATTATTCAATCTGTCATCAAGGCTATGGGTCCCATTCTACACCCAGACTTCTGTTTTCTTCTCCATTGTCCTCC
CTGTTCACTCCTAATCCTCACCCATCTGGTCTACCTGGGAAAGCCATGTAGCCTGGTCTCCAGCCTTCTGGTCTACCTG
GGAAAGCCATGTAGCCTGGTCTCCAGCCTTCTGGTCTACCTGGGAAAGCCATGCAGCCTGGTTCCCAGCCTCTGGTTCC
CCTATTTTCCCTTCAGTGCATCTTCTAAATCTCTGCTGCAATAATCCATCCAACATATTTGTCTAGTGTGTCACATCTC
TCCTAGGCATGTTTCCATGGTAATGAGAATTGCAGTAGAGAAGAGGAAAGCTAATATTTAAAGTCATAACAGACCACAG
CTGCATCTTCCCCTAAGGCAGACATTCCTCTTCAGTACAAGGTTCTTTGTTCAGCAAAACACCTGGGTGGACACAGAAG
GTAAACAGCTCTGGATATAAAACAAAGAGAGCCAGACTTCTCTGCAGAGGAAACAGGGGCTGTAGGTACTGGAGGACGT
CTAGAGAGAGAAGACTATAGATGAAAGCCTTAGAGGGGAAAATGCCACCTCATTATCTCCAGGGGAAGCTGGAGGGAAA
TTAGGCTGGGCTGGGAGTAGGGAATGTCATGTAAAAAGCACAAATGTCTAGTATGAGCTTCCAAGATGCAGACAGTTTT
CCATTGCGCTAAAGCACATTATGCCTATCACTTTACATCTTAAATGAAGCTCATTACACAAACACTGCCATATATGATC
TCTCACTGATGGAAGAAGAGACCTAGGTTCTGTTTGGATCTGCTAAGAGCAGAGATGAAAACCTGAGGTGGGAAGGGGC
CTCTGGGAATTTTCAGTAGGATGGAATTGAAACATGTTCACTGGAGGAGCAGAGGGGTACAGGGAGGGGCAAGCCTGAT
GTAAGTGAAGGTAGGGTTTCAAATATATACTGTTTAAATTTCATGGTGCCTTCAAGTCTTTTTCTCAGAGATAGCCTTA
CTCTGACAGTGTTGCTATGGTGATATATGGTAGCCGTGTTTCCTTGGGACCAGACATAGATTGAGATTTGCAAGTACAA
TCTCTTGTCACCATCTCCTAGGGCCCACTGTGGAGCTGGCATTATCAATTCCACTGACAAGGGTTTATAATGTGCCTGG
AGTCATAGATCTGGAAGACTCTAGGTATTGGGGTCTGAAGTCAGATCAGTCGTCAGACCCTGGAGGATCCCCCTGCTTG
CTCGTCAGTGCTTGCCCTTTATTCTTGGGTAAATGTTCAGCTCCAGGTTTTTTGTTTTGCCTTGTTTTATACTTCATGC
AAACTTTATTTATAAAAGACTCTTAAATTAGTTGTATCATGCCATGCATTCATACAGAATACAGTGTCCCTTCAGGGTT
ACAGAAGAAGAGCTCACAACTCAAAAGGAACTATTAAAAGAGTTTCTAGTGGCTGAAAGTTAGGACATTTTAGGACAAT
TTTACAAATGAGCCATTAAAACAAACAAAACAAAAAAGATATGGAACTTAATTCTGAGCTGAGGGAGCAGCTGTTTAGA
AGTCTGTACACTAACCTAACCCAAAGTCAGGTTTCTGGAAACAGAGCATGCTCAGACTTGAGCCACCACTGCTCCTCCC
CATCTGATATATACAACCAGTTTGTGCCTGAAGTGGCCGCTTCACACTATGGGGCTTCTAGATCAGTAAACTAAAGCTG
CAGGGTGCTGAGAATGGGCAACGTCATTTGCAACTGCTCTTTATGCTCACCAGTCTCTTTTTCAAGTCATCAATGTTCT
GTTGTGGAGAAAGAGGTGGAGGAGGAGAGGGAGGAGGAGGAGGAGGTGGTCATGGCTCCTGCGGAGTGGGTCTGGTTGG
AATTCAGGTCTGAGTGCTGGCGCTGTCCCCGGGAACCCCGAAGCTGTGAATGCTTGCTACGGAGCATGTCTGTGGAAGA
GGCAACTGGAGGAACTGATGGTTTGGAGAGCAAGGAGGCCAGTGGAGGCCGGTCATCCTGCTTTGCGTTGTCAAGACCA
CACCGCTGCCGGAGAAGTTTTAGCCTTTCCAGGTAGACAGAAGGCCCTCGTTCTTCTCCGTTTGTGTTACCTAGAGAGG
TCGGGTGGGCACAATGTGATCTCCATCTGAGGGGAAATGATTGTTGCAGTGGGACTGAGGCCTTGGCTTTCTCTCTCCA
TTTCAAGCACCCGAAGGCCTCTCTCAACATAACTCTGAAAGAACTGTGAGGAAATTTTCAGAAACAGTTCAATGTCAGT
GTCTGAATATTTCTTCTTATATTCATATAACTCTGCTAGGACCTCCTTAGTGATTTTTCATCTATTCGAGAAGCTTGCT
TTTCTGTTTCCTTACCAGACTTGCTCCCAGCCTGGCCCAGGCTATGCTTTGTCCTCTGACACAGGTAGGCCCCCAGTTC
TGACCCATTCTGATTATCAATCATTGTAAGGTGGTCTAGGATCTTAGGTCCTTTTAACTTGCATGTAGAAAGGTCTTTA
GAGTCCTTATAGGAAATTCACTTTTTCACTGCTTCATTTTACTCTTTGGGAAAGACCTTCATGAAAATGTGGGTGTCCA
GAAGAATTTTGTCTAGGTTAATGTTGTTGATGGTGTCAGACAACAGTCAAGCCGTTCTCCACAGATAGAGCTTCTTCCT
AACAAGCTCTGAGAATCTGGGAGAACGAGCTGTTGGTAGCAGGATGGCCTAAAGTAAAACAAGCAGGGCATCGAGGATG
TTGGTCTGGTCTGATTTCTCCCAAACCTTCACCACCAAGAGGTTCACAGAATGAATCACCTCTGTCCTTCCTCCAGATT
CAGCTCCAGTTTTTAAATAACTCCAAGAAAGACTATTCAGTCACAGTTGTGAACCAAACTGCCAGGCCTATCAATTCAA
ACTGAAAGAGAGTACTTCAGGGAAGCACAGGAGGCCATGCTGACTTGAACAGATTTCTTTTTATCCTAAGTCTGGTGTT
CCTTCTACTGCAACAGATCAAGTCCACATCAGTGCCCATGGGTGTGCCATGAGAGAAGTGTCCTAAAGTGAAATAAGTG
TGGAATGACTGAGGTGAGCACAGCTTTTAACATGCTTCTCAGAATCGTCCATGCTAATGTGTATCTGGTTCTCTCAAAA
CACTGGCAGGCTGCGCTTTCTAAACTCAGGGACATGACTGGTAGTGGTGGCACACGCCTTTAATCCCGGCATTTGGGAG
GCAGAGGTAGGCGGATTTCTGAGTTCGAGGCCAGCCTGGTCTACAGAGTGAGTTCCAGGACAGCCAGGTCTACACAGAA
AAACCCTGTCTCAAAAAAACAAAACAAAACAACTAACAAACAAACAAAAAACAAAAACAAACAATGCAAACTCAGGGAC
AAAGAGTTTTCTGGAACAGCAATTAACCTCTTATGAAGCTGGTGTAACACAGAATAGTTTGAGAACTCCAGCTTTCCAT
GCAGAAGAATGCTTGATTTGAGGGAAGAACACTGCGAAAAGCCAGAGCTTCTGCTAACATAAACTGACTCTGGAAGAGG
TTTTATTTTTGCAGGAAGAAGGGAATCTCCAGAGTCCTGGGTGGAGATGGACTTCAAAGAAAAAGAACATTGAAGCAAC
AAGTCATGGGATAGGCTTGGAGTAAATACAACTTAGTGGGAGGGGTCTGAGAGCAAAGCTAGGTATCAAAGAGCACACA
CTAACTAGCACTAACTAACTAACTAACTAGCAAGGATCGCTCTAAGCACAAGAATGATGGCACCTTTGTTTTGTTTTCT
CCAGGACATAAATCTGTGTCTGAGGCCCACTCACCACAGTCTGCAGCAAATCCCATGACAGTCATTTCCTGGGTGTTCT
CTCCTTCTCTTAGACCATTCTGGAAACTAAGCTTGTCAAGTCAGTAACACTTGAGGGAGATGAGTTCTGGTCTTGGGGT
AGCCCCAGGTTGGTCACTAGGATAATAACCCTTGCTGAATTGTGGGCTTGAGCTTAGAATGCCATTGTGTCTCTAGTTA
GGAGTCCGAGATGATCCTCCTTGTGCTGTATCACTCACACACACACACACACACACACACACACACACACACACAGAGA
GAGAGAGAGAGAGAGAGAGAGAGAGAGAGAGAGCCTTGCAGCTCCTAGGGTATCTACGCAGTGTCAATAGATATCAAAG
CCCCTCATAGTATCTGTTATGCCCCCCTCAAAGCCTCAGTCTGCACGGTCTACACCTTCTTGCCTGGAAGGTTCAATCT
CACCGCACCCCAGCCTCACTACTACTCCCGGTACATCTAATTCACCCCCTTCAGCTTTTGAATGTAGAGGTTTGCAGAT
GTTCTTCTGGGGTCCAAGAGAGCCAACCCAACCTTGAGCATGCAAAGGCAGCATGAGCTAAAGAGTCTTATTTATTCTT
TATATAGGGTCTTACACAGCCATAGCCCAGGGGTGTCTCGAGCTTACTATGCAGTCAAATATAACCTTTAGTTTACAGT
CTTCCTGCCTCTACCTTCCAAATACTGGGACCATAGGCAATGTCTCCATGTCTCACAGGTCTGCTTTATGCTTTGTAAA
GGGTCAAACTGCATGCACACTAGGAAGGCACTCTACTGACTAAACCACATCCCCTGCTCAGTAACCCTCCATGGGGGAT
GATGTTTCTTCTTTCGTTGGCTAAAAAATATAGCAGATATCTCTCTGAAAGCACCAGTTTTTAAAAGGTAGAGTCTTTA
AAAAACAAAACAAAACTAACTTTTATTTATTCTTTGTGAGTCTCACTTCATGCACCCAAGTCCGGCTCATCTCCCCATC
CTTCCATATCCACCCTTGCAACCTCACAACTAAAGAACAGAATGAAAAAGAATCCATCTTGTTTTGGAAGCTGCAAGTG
TGTCATGCAGTCTAGCCTTTGGTTCACACACTTGCAAATGTTAATTGCATTGAGTCATTGGTCTGGTTCGAGGCCTCCG
GCTTCTGCTGCACTTTCAATACTGGGTCCTCACCTAGACTCCTCTCAGACATCCTGTTGTTGCCTTGGGTCATGGAGAT
CCTGCAGTTTTGGTTCTGCAGGACCGGCCCTTTCATACCCTCCAGCAGTTCACAGATGGGGTAGATGTTGGGGCAGGCC
AACTCGAAGGTCTGGATCTGGGCCTGGGTGGCGGCTGAGTTGGTCTGTGTTTTCTAAAAAATGTATTATATCACAAAAA
CCCAGGGAAGACTTTTATAGTGGCTATCATTATATTTTTATTATCATTATATTGCTTTGAAACAACCTAGTCTCCAAAA
TAAAAAGCAACAAAATTCCCCAAACCAAAAAACCCCAACCATCTGGGGGTGAAGAGCAGGACTCAACAAGAACAATAAC
AAACAAACAAACAAAGAGGCAGACAGACTCTGGATTACACGGAATGTAGTTCAGCATGTGACTTACTGACAGAGGTGAG
GTCCTGGGTTGCAACAAAGCCAACTGGATACTCACAGTTTGGGTCAGAAGGAGAGGTGCATATGTTGGTTAGGACAAAA
GTGATCTCATTAAAGGGGAATGTACCTGGTTTATCACAAGCTAGTATCAAACAGACAGGCGCATTCCTAGCACCATGGC
CCAGCTGTAAAACCGCAATGCTTCTCTAATGGTTGTGTCCTTTTACAGTTGTCTGGGAAACTATGTGGGAAACCCCAAC
CAGGTTATTCTGGGGCTGATCCTGTCTGCTACAACTCAAGATGGCTGCCATTATGTCAAGATGAATTCATTCAATAACG
AAGCCAGCAAAAAGAAATAGGCTTCAAGGGCGCCCTAGTTCCTGTGGGAAATCCTTCTGTTCACGTTTTGGAGGAAACC
GTGGAATGATTCTGACGTGAGTGACAGTGACAGGCCTGTGATCTCTGCTGTAGCTCAGGTGTCTGGAAAGTGTCACTGC
TGAGCACAGGAGCGACCATGTTGATAGCAATGGCAGAGGATAACGTAAATACGGCCGTCAAAGGAAGGCTGCCATGATG
CTTGAAGCACGCTGACTGACGTGATAGAACTATGGTGCCCAGTTGTTTGGTAAACACCAGTGTACCTAACGAACGCCAT
GAAAGTATATTTTAGATGTAATTAACTTTCAAATCTGTAGACTCTGAGATTGCCTTCCACAGGGTGGGTGGGCCCTGTC
TCATTAGTTGTAACCCTAACGGCAGAGACTGGTTTCCAGACAACAAAGAAATACGGGCTGAGTTTGTAGCCCACAGGTT
TCAGCCTCAAGGCTTGAATGTTAACTATTCCCTCAATGTTCTTCTTTTCTCTTTAAAATTAATTAATTTATTTATTATG
TACATTGGTGTTTTCCCTGCAGCCATGTCTGTGTGAGAGGCTTGGATCCCCTGGAACAAAAGTTTGAGGTAGCTCAAAA
GTCATGAGGTACCTTTGGCCACTGCAGGTTTCAGCCTTACCTGTCCCACTGTGAACTAATTCCTTAAAAATCCCTCTCT
GTCTCACTGTGTGTGTCTCTCTTCTGTCTTTCTCCATATGTGTGCATTTGTACTATTTTGAGCATGTCTGGATTCAATA
GGTGAAGAATTCAAGGGATTCTGGGATGAATGGGAACCTGGGTTTCGTTCCTGACACTGCAAAAACAAACAAGTTCATA
CATATATAAATAAAAATGAAAGAGAAATTAAAGGATCACATAGAAGCGGCGAGCAAAGAGGGGTGCATGCTTATTATCC
CCTCCCAAATCCCAAATCCCTTTCCTGACGTCAGGGACAACATAGGAATGAGAAGAGAGGTGGGGGGGGGGGCACGGGG
AGGGCAGGGAGGAAAACTTACAATAGAGGCAAAAAACAAAACAAAACAAAACACACACACACACACACAAACAAAAACA
ACAACGACAACAACAAAAACCAAGAAAGTGTTCAATCAGTGCCACAGAATTTCTGGGTTCTGAGAATCCTAAAACAAAC
TTGAGGAAGAATATTCACCTGGATTTGAAAGTCTAGAAAGTACCACAGGTCGGCAAGGAAGTTCTCTTTCGTTCTGGTC
TGAAATAATTTCCCCAGGCAGCCATTCTCTGTTTGTGTTGGTGGCAATGGCACGCTGTAAATGCTGTAAATGCAAGCCT
TCAAACTGTATTTTCTCCCCCTTCTTGCTCTACCTTCCCTTCCCTTCCCTTCCCTTCCCTTCCCTTCCCTTCCCTTCCC
TTCCCTTCCCTTCCCTTCCCTTCCCTTCCCTTCCCTTCCCTTCCCTTCCCTTCCCTTCCTTCCTTTCCTCCTCCTCTAC
CTTCCTTTTTTTTTTTTTTTTTCTCGAGACAGGGTTTCTCTGTGTAACCGTGGCTGTCCTGTAACTCCCTCTGTAGACC
AGGATGGCCTTGAACTCAGAAATCCGCCTGCCTCTGCCTCCCAGGTGCTGGGATTAAAGGCGTGTGCCACCACGCCCGG
CAGCATTTTTCAACTTGATACAAACAAGTCATTTGGGGAGAGAGAAACTCAACAAAGGAATCACCTCCATCAGATTGGC
CTATGAGCAACTCTGTGGGGGCTATTTCTTGATTAATGATTAAAGCAGGAGGGTCCAGCCCACTGCAGGCAGTGCTACC
CCTGGGCAAGTGGATTATCTACGAAAACAAGTTGAACAAGTCAGAGAGATGTTTCATGGTCTCTCCTTTAGTTTCTGTG
TCCAGGTTTCTGCTTGAGTTGCTATCCTGAGTCTCCTCAATGATGAACTATGCTTCTGTGAAAGGGTAAGATGAAATAA
ATATGTTGCTTTTGGTCATGGTGTTTCGCAGTAATAGAAAACAGAACAGGACAGTAGCTTTGCTAGTACACCTAAGGTG
CACACACTCCTGACTGAAGAACGTAGGAGAAGTGGAATAGCTGAACTACAGAGAAGACGCTCTCGTGAAATGGATCAAT
GACTGCCAAACCGTCTGCATAGGATACCAGTGATTTCCACTCAACCAATTATAAAAGTACCAATTTCCCCACACTGTGA
CGAGTACCATGTAGTCAAGCTGTTAGAGACTGGACCATCTAATCATTAGATTAGATTATGATGGGTTAAAGTATGGGCT
ATTGATCAGGGAAGGTTAAATTTGTATTTTTCTCTTGACTGTGATGAAGGAGGCTGGACAGACATTGATTGCTGCCCCA
AGGTTTGATTCCCAGCATCCACATGGTGGCTCACAGCCATCTGTAACTCCGGTTCCAGGGCATCTGACTTTTTCTTCTG
TCATCTGTGGGCCCTGCATGCAAATGGTGCACAGGCATACAGGCAGATAAAACTCACATACATATATTTAAAATATTTT
TAAATGCCTCTAAAATTAAGCATTTTAACACATTTTCAAATTGTTTTCATTTTTCTGTGAATTGTCTGTTTCAATAAAA
TATATTTTTATTGTTGTTGAATCATAGGTATTGTTGCATGTGTGTGTGTGTGTGTACATGTGTACACCAGTGTCTGTTT
ATGTCAGCTGGCAAAAGTTCCTCATAAAAGACAGTTCTTTGACGGTGGTGAGACTGCGAAGATTTCCTTCTGTCTGTCT
TCTGTTTTTTGCCATTGTTTATAGTGATTTTGTCATGCAGAAACATTTTATTTCTTATGAATGGATAACACACACGCAC
ACACAAAATGAGAAATCATGTAAAAGAAAGCAGATGGTATCAAATTAACAGCAAAACAAGATCAGAGAAAACCACATCG
TAAAACACACCCAGAGAAGGTCCCACTTTGGAGGTGGGAGTGACTCTGGGACAGCTCCAAGTTCTGGGTTAAGGGACTC
GGGGTGGGGGTGTGGGGGGAGTTGCTCCTGGGCAAACAGCAGCATATTGAATGTGGAGTGTCTGCTGGAGGAATCTTCA
TTCTGTTTGTTCCTCCTGTACTGAGCAGGCATTAGCAGCAGAGTTGTGCCCTGGGGACTGTGAGCTGGGACCAGAGAGG
TAGAATGATGTCCTAGCAGCAAGTAACCCCAGGACAGAGAGCATGTGGACAGCAGAGGGACCAGGTTCTCCCCAGAGCA
CACCGCTATCTCTAGAACCTGCACCTCCAAGACTAAGACACTCATAGGACACAAGTGGCTGGGGAGATAGTCCAGGAGT
TAGGGACACTCACTGCTCTTCAAGAGGACCTAGATTTGTATCCCTGCACCCACGTGGTACCCACAACTGTCTGTAATTT
CATTTCCAGGCTTTTCTGGCTCCATGGGCACCGGTCATAAAAATGATGCTCAGACTTACATGCAAGCAAAACACCTATA
CAGAGATTGCTTTTTTTTTAAAGTTTGCAGTACACAAGACCAGGGTGGCAGATAATCAATGGCTAGCAGATCATGGATG
AAACCAACATCCTGAATGAGAGTAGATACTGTTTTTTTTTTTTTTTTCCCTCACAGTCCACCAGTGAGAACTTAGGTAA
ATTGCAAGGGAGGCTGGGAAATCAGCCTAGCCAAAACCTTTATGAGTGTTATCAAAGGGGAGAATGGATTCAGGGGACA
ACTCGCGCCTGTCAGGAACACAAATGTCGCTTCCCTTTGGCTCTAATCATATCCCCAACCCAGGGTCCAGGTGGTCACC
CACTTACATCTTTTTAAGATACTTGAAATAGATCATAAATATGGACACCTCCTACTGTCAGACCTCCAGGTTTTCAATC
TTTTGGCAGACAGCGCTGCAAAAGTTATCTATAATATAATCTCTTACACAAATATACAGATATATGCATAAGGTCAATT
CCTTGGTGCTGAATGTACTGGGTCAGAAGGCACACACCTTTACAGCTATGATGCCATTGTCAAACTGCCCTCTGGAGAA
TTTGCATCAGCAGAAACCTCAAGCAACAATGTCTTAAAGTCTCAGTTTCTCCACATTTGTGCCAAAAGAATGTATTTTT
TTAAAAAAAGAAAAACCTTTTGACTTTTGGTGTACTGGTGAGAAATGAGTCCTCACTGCAGTTTTAATATGATTTCTAA
CCCAGCCAAGATTCCTAGTTCTTTTTTAGTATCAGAAATAGGCATCTCCAGGCTTTAGCCCAAGGCACGGAGTAGGACA
GATCCTCTTCCCTTGTTTTTCTCTGCCCCCTTTTCTAAAAACAAGGAGGGGAGGGGAGGGCTGGAGAGAGGCCCAGCAG
TTAAGAGAACTTAGTGTTTTGGCACAGAATCTGAGTTCAATTCCCAGCACCCACATGGCTACCTTACAACTGTTGATAG
CTCCAGTTCCAGGGGATCTGCTGCTCTCTTCTGACCTCAGGCACCAGACACTCACATGGTGCCCAGACATACATGTGAA
GAAAATGCTCATTCACAGATGATAAAAGAAATCTAGAAGCTGGGCGTGGTGGCATATGCCTTTAATCCCAGCACTTGGG
AGGCAGAGGCAGGTGGATTTCTGAGTTCGAGGCCAGCCTGGTCTACAAAGTGAGTTCCAGAACAGCCAGGGCTACACAG
AGAAACCCTGTCTCAAAAAACCCCAAAAAAACCCCAAAAAACAAAAAAGCAAAACAAACAAACAAAAAAAACCATTAAA
AAAAAAGAAAGAAAAGAAAGAAAGGAAAAAGAAATCTAAAAATAAAATTGAAAATAATGACTATGCATGCGCATGCATG
TATTTGTGTACCCAAGTGTGTATGTGTATGTGTGTACACGCACACATGCACACATGTAGAGACAGGGCTGACGTTTGGC
GTCATCCTCAACTGTCCAGCTGCTTATCTTTTTGGAACAGGTTCTCTCACTGAACCTATGTTTGCTGACTTGGCTGGGG
TGGTTGGCCCGCAAGCCCCAGAGATCCTCCTGTCTCTAATCTCCTAGCTCGGGAGTTAGAAGCCTGTCCCACCACACCT
GGCTTTTTATGTGGCTTCTGGAGGCTCAAACCCAAGTCCTCAGGCTTGTGTGGCAAAGGGGCTCCCTTTGCCAAAGGAG
CGGAGTCCTCAGTTACTTACTTTGATCAAAGTTGTAGCATTTTTGTACGTTTTTAAAAAATATATATACTCTGGGCCGA
CTTTAATAACTTTGGTGTGTGTGTGTATGTGTGTGTAATGTTTTAAGAGATAAAAATCACCTAGATACTGTGGCTGTTA
TAATCACCTACCTCTTTTAGTTTCTGCTATTGTGTAACTCACGAAACTGGTCAACTGTTTAAAGTACCATGAAAAATGT
AACCCATTAAAATGAAATCCTAAACAATCTGGAAGTCCAAGCAAGACCATCACTCCCTAGGAGAAACACACATGAGCTA
TCTTATGTGGCTTTCAAGAGTGGGGAAAAGAAAGAAAGAAAGAAAGAAAAGAAACAAGTTGGGGCTGGAGAGATGGCTC
AGCACACCTGCTGTTCTTCCAGAGTTCAATTCCCAGCAACAATTTGGTGACTCACAACCATTTACAAAGCAATCTGATG
GCCACTTCTGGCATGCAGGTGTACATGCAGGTAGAGCACTCATACATAAAACTATAAATCTTTTTTTAAAAAGAAGTGA
GGGCTGGAGTGATGGCTCAGTAGTTAAGAGCACTGACTCCTCTTCCAGAGGTCCTGAGTTCAAATCCCAGCAATCATAT
GGTGGCTCACAACCATCTGTAATGAGATCTGATGCCCTCTTCTGGTGTGTCCGACAACAGCTACAGTGTACTTACATAT
AATAATAAATAAATCTTTTTTTTTAAAAAAAGAAGTGAACTAAATTTCAACAGTATAATTTAGCACAGAATATTTAAAA
TTGTATCACTGACATGTAACCAATATAATATAGATTAATGACACTTGCACATTCTTCAAAATCTGGCATCTACTTCACT
GCTAGCCTATTTCAGTTCCTACCAGCCATATGTCAAACACTCAGTCACCGTGCGTGCATGGCTAAAATGTCTGGTGCCA
GGGGTGGAGTTCCGGAGAGCTCTATATGGAGGTGGAAGCAGGTGAAAGTTCTTATTTTACTTAAAATTCTGCAGAGCTA
CTGAGTGGAATATGCAATATGTGATGTATAATACATGGTATTTTCGACAAACGGCTCCCCATATGGAAAAAATAAAATC
GTGTCATGTGCTGTGCATAAACACACGTTCCAGATGGACAAAGAAACGATTTTTTAAAGGAGAAAAGCACAAAAGCATT
AAAAATATAAGCGTTATTAATTGGAAGTTCATTCCGAAGCACAGACACAAGATACAGAAACCATAACGGAAAGGATCGT
CGGTTATGGAAGAGAAAAATCCAAATTTTAAGCAAAATAGTAAGCAAAGTTAAAGGACAATGGGTAAAGGAGAAGGAAT
ACAAATGAGATAGAAGACAATCAGCCAACCAAAACCCATGCAAAAGTTGCAAATATTTTATTATAATCGGTATTTTATT
TATTTCAGTGTGTGTGTGTTGGGGGGAAAGGGCGGGGGCGCATGCGCACACATGTGTGGAGCACTCGGTCATCTTTTCA
CCTGTACATCAGCCCCAGGGACAGTCTGTTCCAGGGACCGAATCCTAACCATCAGGCTTTCAAGGCGAGCATTTTACCC
TCTGAGCATCTCACAGCCCACCCTGAAACCTAACACTTTAAAATGTAAATTAAAGTGCATAGAAAATTCTCTCTCTATA
TATTGGCTCTATGAAGGACGCACATGTACAGAATGCTCAAGCTCACCAGTTATGGAAGGCAATAAAGATCAAACAATAA
TAAACCAATTTCCTTTTTCCAGTGGCTGTGATAAAACAACAACCAAACCAAACAACACCGACAAAAGACAGCAGCAGCA
ACTACAACAACAAAACACAAAATAACTTGGGAGGAGGAAGGGTTGATTTGGCTTACAGCTTACAGTCCGCCACTGAGGG
GAATCCAAGGCAGGAACTTGAGGCAGGAACTTAAGGCAGGAATGGAAAGAGACACCATGGAGGAATGTTATTTAGTGGT
TTGCTCAGTTTGCTTTCTTATAAACCCAGGAAACCACCCACAATGGGTGAGGCCGAGTCTCATCGATCATCAATCAAGA
AAAATGCCCTGTGGACGTGACCAAAGGCCAGTCTGATGGAGGCAATTCCTCAGTTGGTGACTCTAACTGGTATCAGGTT
GACAATAACTAACCAGTACAATTTACCTATTAAGCTTTCTGGTAAAAAAAAAAAAAGGCACTTTGGTAAAATCTTTTGA
AGGGCAACTGAACACAATATATATATTGAGTTTGAGGTCAGCTTGGGGGTAGTGGCCCTGTCCTCCCCCTAAAAAAAAA
AACCCAAAAACCCACAATTTATGTGAATCATGATTTTATATATGAAGTGGAAAAAAATTAAGCCCAGCTGTGGATCTAA
TATATATGGATGTGTAAATCCTACACAAAAGACTATGGGAATAAGCACTGAGTTGGATGTTTAGGAAGAACAATCAAGG
TGTACGTGAAGAGGAAAGTTCATCTTCTGTGCTATGGACTTCTCTAATGCTTGATCTGTTTATAGTGGGACTGGGTTGG
CTTACGTGAGGAAAGAGATGGTTTACAGAGTGGCCTTCTTAGGTAATCCAGCAGAGGACCCTAGTGGGAATATAAATCA
GTCAGGGGTAGGGGTGGGGCGCCCCTGGCTCTGTGAACTCTGGGCTGCCAGAATATCTTTGTGGGTGCCAACCAGTGGA
GATGCTGAGGCTATGCCAGCGCCATGCATGAGACACCTGTTACTTCATCTCACAACCTGTGACCAGCCTGCTGTGTTTC
CATGCATCCATCCTGTGGTATTTCAAGCCTAAAGCTGGCCTGTGTCTAGAACAGTGAACACCAAGTGTTATGTCCTGAC
TTTTCTTAACTATGTCTTCAGCCACAGTGACAGGAAAAACCAAGGAGGTTGGCGGTAGGTGGGCAGAGTGTGGTCATGG
CCGTGGCCTCCATTTTCCCTGGGGACAAAGAAACCTCAGGCAGACGTCAGAACTTCCCAGCATCTAGATGTTGAGGCTT
ACTGGGAAATGTCAGAGACTGCCACCCACACCTTGGCAGGGGAGAGAAGAGTCTTAGAGAACTCTGTCTGACATAGGAA
GAGTCTGGAGTCTGAGAGTTCAATAAGAGTGTTGCAATTTTCTTCTTATTCAGAGATCAATATGTGAATATAGATGTGA
AGGAGGTGGGAAAAGAAGCAATAAAGATGTTGGCAGAACAAACATTCCTGAGGAAGGATTGCGCCTAAGACTGGAAGAG
AGAGAAAGCCATAATTGAAGCCATGGTAGCTGAAGGTAGGAGATACAGTCAGAAGGCAAGGGAGAGGTAGGGCTTGAGA
CCCGCCTTCTCTCCTTGCAAGAACTTTGCTTTTATTCTAGATGAGCCAGAGAGATATTGGCAGTCAGAAGGCCAGTGAA
TAATGCATCCGGCTGGTGTTTTACAGGATGGCTTTGGCTGTTGGGAACAGACTGAGTGAGCGAGACAGTAGCAGGCAGG
TGCCCTGGGGGTGGTAGGGGTGTGGGGGTGTGGATACGGGCGTGGGGAGGCACCCGTAACAGATCAAAGGAGAGGCAGG
AGTTGAGCCTTTCTTAGCTGAGGGGCCAATACCAGAATCACCTTTAGGGTTCACGGAAACACAGGTTGGCCCCCCAATT
CCAGCTGTTCCTCTCCTTATCTCCTTAGTCGGGTTTGAGCCTAGGACTTTGCATTTCTATAATTCCCCGGTGAAGCAGC
TGGTTTGGGAGTCACACGCAGAGACCCTTAAGTTTTCCCCCATAGCACGGATGAAAGCTCACATCCTTGTAAGTACTGA
AGGAAGCTCTTTGGCATTCCTGCACCCTAGTGTGCTTCATACAGCAAAAATTCATGTTGGGTACAGGCTCTGGGTTATG
CAGGGGGGGTTAGTGAAGGGACTTTTGGAGGATACCTCATTTGAACTGTTTGGTGATGAAACTACACAGAAGTGGCCAT
TAGAAGGAAGGGTTCATTATGTCAACAGTAATCATAACAATTGTAGCAGTCACCCAGTGTCCGCCCAGGTGTTTTATTT
ATAGCTTCTCCAATTAAATCCTTGTAAAAGCCCTGCAAGTCAGGCCTTACCGCTGGTGCTTAACCGAGGCTAGGAGAGT
TTAATTTTCCCAAGGCCACACCATTGATAAATACCAGTGCTTAGTCCCGCTTGACTGACACCTTTGGGATATATTGTAT
CATAGTATCTCAAGATGTCACTGGCTAAAGATATGGAAGTAAACCAGCCTCTAGAAGACTCAGCTTCCATTCTTGGAGA
TTATTAGCTTGCTGCAAAAAATGTAGACAGCAAACCTTTGCTTTCAAGACGGCTCCGCCCCCACCCCCTTTGTTTTCTT
GAGTAGAGAGCCCTGTTCACAGCATAGGTTCCTAGAGTCCTTCCTGGCAGGGCCCTGGTTCCCTACGAGGTAGCTGTGG
CCACAGGTGACTTGTGTTTTTCATCATTCACTGATGGCATGCTTTTGTTCAGTTCATGCCCACTTCCTGGTCTCTTCCT
GCAGGGCACTGTATTCTGTGCTGGGGAACTGAAGCTTGACAAATTGAGGCCAGCTTGTCTCCAAATGGAATCCATCCCT
CTCACTACAGGAGAGAGGGGGGAAAAATATCAAGATCCAACTATCCTCCTCTTCGGTCTCTCTCCCTGCCCTGGCAGGG
GATACAGGTAATATCTCTCAGGGCCTGCGTGTGGATGGGCAACTGGGAGCTTGAAGCTAGGCCGGGCAGAAAGCAGCTG
CTCTCCACAGGCAAGGAGGGAGGCATAAGGTCAGTGAACGGGTCACAGGGATGGGATGACCCTGTTCTGGAGGAAGTGA
GAAGGGATGCTTGGGAGCCAGCCTGAGTGATGGCTTGTGGAGTAAGCTTGACTGGGGCTTCCCCAAATTCTTCATCAGA
GTCTCTGGGATGTATCAGAGATCCTTAACCGGTTTAGGGAGACAAGGGCTTCCTAGAGCAAGGGATGAAATGTAAAAAC
CACTCTCCAGTATGTGAAAGCACCCATGCACACAGACAGCCATGTACAGCCATAAGCACACACTCACATACATTCCATG
TTTTGGAGGGGGGGGCTGGCATGGACCACAGGTTAAAAGCCCAGATACAGGTTGATTTACAGTAAAGGAGAACTAGGAT
AATAAAAGTTCGTCACATGTAAAAATAAGCTTGCTGGGCATGGGTGGCACAGGCCTTTAATCCTGGCACTTGGGAAGCA
GAGGCAGGCAGATCTCTGTGAGTTCAAGGCCAGCTTGGTCTACATAGCAAGTTCCATGCGGGCCAGGGCTACATAGTGA
GACCCTGTCTCAAAACAAACAAACAAACAAACAAACTGTAAACCAAAATACAAAATAAAAAGTTTACTAAGGTTTTATG
AGTTTTTTTTGTTTTTGTTTTTGCTTTCTGGAAAGTGTGTGGCATGTGAATTTTAGGGAACAGACATGCTAACTAGAGG
CTGACCTCTGATGGTGGGATAAGGGAAGATTTCTACGTACCCGTCCCTTCTCACTAAATAAAACAGCATTCTTTTCTTT
TGAGACAGCATCCAAGACTCAGGGCGCACCTGGGGAAGAATAAAGAGACCAACCGACTCTAGGCCTCAACGACTTGGGC
TGGGGACTGGCTATATTAGCCTAGAGCCCTTCGCCTGGCCAGGGCACTTCATTATTGTTTTTGATTAGTTTGTGTCTCT
TTAACTCATTTCCTTTATTCCTCGCGCGTTTCAGCCTGGCAAACAACGTAATGTTATTTATACCAACCCCTACGCGCGG
CCCCCACCCCGACCTCAGGTCTGAGACGCTCTCCCGTGCACGCGCGCCCCAGAACCAGGCGCTCTCGGTCGGGCGGCAC
ATGCGTGCGCGCCCTCGCGCACGGCGGGCACGCGCACGCGCGCGTCTCCGCCCGCAGCTCGCCCCCCCACCCCCTCCCG
TTCCCGGGCGGCGGGTCTGAAGCGGCGGCGCGATGAGCTGGCCGGCCGCCCCCGGCTCGGGGCTGTGAGGCGCTCGGGG
CCGGGGTGCGCGGCGGCGCGGCGGGCGGCGGACGCTCCTGCTCGGCGGCGGCCATGCGCGCGGGCCGGGGCGGCGTCCC
GGGGAGCGGCGGCCTGAGGGCCCCGCCGCCGCCGCTGCTGCTGCTGCTGCTGGCGATGCTGCCCGCCGCCGCCCCGCGG
TCCCCGGCCCTGGCCGCCGCTCCTGCGGGACCCAGCGTGAGCCTCTACCTGAGCGAGGACGAGGTGCGCCGGCTGCTTG
GTGAGTGGCGCGGCTCCCGGGGCCTCGTGAGCCCGCGGTGCTGCCGCTGCGCCGCCCGCGCTCTCTCTCTCTCTCTCCC
TCTCTGCAGCCTCCCTGCGCGTGTCACTCTAGGCCGCGGAGCTCCGGAGCCTCGGAGTCGGCGCTCGGGACACGGGTGG
CCGCTCCTTAGCCTGCTGTGGGTGGCCCACAGGCCACGCCGCCGCCGTGCGCTTGCTTTCTGGTTAGCGAGTGGGGGGC
AAGGTGAGCCGGGCGCCCTGAGTTGTGTGGCATCCTGCACACCTTCGGGCCGACCGGATCCCAAGCATCGGGGTTTGCC
TTGGGTTCCTTGAGTGTGACCTGATGCTGCCGTTCCAGGCCACTTGCCTACTCTGCAGACCCTGAAGCGAGAGGAGAAC
TTGGCTTTGGGGTCCGTCGGCGGTGGTTCCGTTTACCTGTTTAGCCCGTTGTTTGGGGAGTCCGACTCTGCCCTCCGCT
TGGAACTCTGTGCATTCTGAAAATCAGCCCTTGAAGGAAGGCCTGGCCAGCCATCAAAAGGAAGTCTCTGATAGTTAGG
GATACCCAGCTTTTGCCGTCTTTTGTGCATTAAGGCAGTCCCTTCACTTTTTATCGGGGTGTTTTAGAGTGAAGTGTTG
GAACTGGAAAGGTCTCCCCCTAAATATCCATTAAGTGTCACTTTTTTAGTGAGCGAGTATGCCTCCCCCAAGTGTGTGT
GTGTGTGTGTGTGGTGTGCGCGCGCGTGTGTATGCGTATGTGTGTATTAGAGGAGGTTTTTTTGTTTGTTTGTTTGTTT
GTTTGTTTTGTTTTGTTTTTCCTTTCCAGACAGGGACTTTATGTAGCCTGGGCTGTTTTGGAATCCACTATGTAGACCA
GGTTGGCCTCAAGCTCACAGAGAGCCTTCTGCCTGTTAGAAGAGTTTCTTAGGGTCTGTTGAGAGAGTTTAGCCTCGGA
ATGAAAACTGCTTTTAGCAGACTGTTTAGACATAGTTGTATGTGTAGGTTTATCGGCTTTAACTAAAGAACTGTGTTTT
CCTAACATCCTAATTTAACCTTCCTTATAGTCTAGTTGAATTAGCTGAATTTAGTCTAGCAGAATTGAAATGGCATCTG
AGTTACGCCTGGAGCATGGGAGAAAGGCTTGGGAAAAAATAACGATTTGTAGTGCGGCTCCTAGGCGTGCTTTTGTAAG
GGTTGTTTTTGTTGTTGTTAAAAGAACTTACGTAATTGTGACTTTATTGACATGTATTTCCTCTCCTGCCTTAGCCAGG
ATGGAAGTAGGGAGACAGAGATATCTGGCAGCAGACAGAGACGAGTACATGTGCCTACCGTTGCGTGTTAGAGTTTTGC
TGTTACTTTTATTTATTTTGGTGTGTGTGTACCACGATGTGTTCTGTGGAGGTCACAGGACACCTTGAAGGTGTGGGCT
GTCTTTCCACTATTAATAGGGGGTCCCAGGGCTCAAACTCATGTCATCTGACTGGACGACAAGTGCTTTTACCTGCTGA
GCCATCTAGCTGGGGCTTGATATGGACTTAAACATGTTGTTTCAGTTTCTTGTTACCCAGTGGGGCAATTATGGGAGAT
GAGAAAAGTAAGGCTTTCAGTAACTTGCTGAAGGTAACATGGTTTATTTCATATATTACATATATAATATATACCCACA
CAAGACAGTGTTTCCTGGCTGCAGACTAAGTTATTGAAGGGCATGTGCATTTAGAAGTTTGATTATGGCATGTTAAAAA
AGAGACTGTTGTTCATTAGTACGTGAAGTGATACCTTTGTAGCCTGGAACTGATTTTCTTAGGACAGTAGCTGAGGGGG
CACCTGATGGATGAGCACCTGGTCCAGTAGTGTGTGAAATAGAGTGGGGAGCATGGCTCATTAATGCTCCCATCCACCC
TTAGGTATTTCAGTTCCATGCAAGCTGAGTTGTCCTGTTACAATGTGGTCTCTAGATAGTCCTCTTGCAGAACCAGGGC
CTCTTTCTGTCCCTTTACGTTTCCCTCAGCTGTGCTGCTGTTGAAGGATTCCCATCTGGCTTTGTCTGGGTGGCAAAGT
GCAAGTACTGGGACGTTTGCTGGGCCTCCTGAGTGTGGCTGTGATGGTGTGTACGTGCGAGACTGTTGGAGATAGTGCT
TTCCACCACGTTTTCCTTTGCGTAAAGGGTTAAGTGACTTCCAAAAGCTCAGGCAAGCTAGTGTCAGTTTTACAGTGTT
GCTGAGACCCAGGGTTCAGAATGCCAGGTGAGAACTTGGAAGTTGGAAGTGACAACTCTGTGGAGCTGGCTGTACTTGA
TAGGAGAAGTGCTTTTAATCCAGACATTAGCTGCACATAATAGAAATCAATTGGATAATAGCAAAAAGGCCTTGAACCA
GAAGCCATCAGCACCCTGCCTTTGGGCTTTTTGCCCTCCAGGTCCCACTGCCCCAGTGGGCGTAGTTCACACAACTGTG
GTGGAGTTTGTCTTTCCCTTGTTGTCTTTGGAATTTCTAGACTAGTGTGGACTGAACATTTGTCTTCTGGGGTCCTCCT
TGTTCATCAGACTCCCCAGGGCAGGTGGGTTTTTTTTTTTTTTAATTTTAAAAAACATCCCAATCACAAAGGAACTCAC
ACAATATCTACTCACTGATAAGTGGATATTAGCCCAGAAACTTAGGATACCCAAGATATAAGATACAATTTGCTAAACA
CATGAAACTCAAGAAGAACGAAGACCAAAGTGTGGACACTTTGCCCCTTCTTAGAATTGGGAACAAAACACCCATGGAA
GGAGTTACAGAGACAAAATTTGGAGCTGTGACGAAAGGATGAACCATCTAGTGATTGCCATATCCAGGGATCCATCCCA
TAATCAGCTTCCAAACGCTGACACCATTGCATACACTAGCAAGATTTTGCTGAAAGGACCCAGTTGTCTCTTGTGAGAC
TATGCCGGGGCCTAGCAAACACAGAAGTGGATGCTCACAGTCAGCTATTGGATGGATCACAGGGCCCCCAATGGAGGAG
CTAGAGAAAGTACCCAAGGAGCTAAAGGGAACTGCAACCCTATAGGTGGAACAACATTATGAACTAACCAGTACCCCGG
AGCTCTTGTCTCTAGCTGCGTACGTATCAAAAGATGGCCTAGTCGGCCATCACTGGAAAGAGAGGCCCATTGGACTTGC
AAACTTTATATGCCCCAGTACAGGGGAACGCCAGGGCCAAAAAGGGGGAGTGGGTGGGTAGGGGAGTGGGGGTGGGTGG
GTATGGGGGACTTTTGGGATGGCATTGGAAATGTAAATGAGGAAAATACCTAATTAAAAAAAAACATTTATTTTTATTT
CATGGGCATTGGAGTTTTGCCTGTATGAGGGTATAGCATCCCCTGGAACTCGAGGTACAGACAGTTGCGAGTTGCCATG
TGGGTTCTGGGTATTGAACGTGGGTCCTCTGGAAGAGCTGCCAGCACTCTTAGCCACAGAGCCATCTTTAATCCTCCAG
TGCATGTTTAAACCAGCAGTCAGTTGCCACAGTGGTCTGGTCTGTGCCCTCAGATGGTCAGTATCGATGCTATAGCGTA
CAAATACATTGCCCTTTTCTGAGTTCGATATGAACTGGTAGACACCCCCTCCCACACTCACCCCCTTTGTTCGGGTTTT
CCCTTTGGATCTTAAACTTTTTTTTGATTAGCTTGCTTTGTGTCTGTACATATATTCTCTCGGGTACCCCAAGAAGCAC
GCTGAGTGCCTTTTGATCCCCAGTCCCCTTCAGGCAGCACTTTAAGACATCTGTTAGTTCCATTGGATTCTTCTCAGTG
GTACCTTCCTTGATTTAGTGTCATCCATCCCTTGCTTCAGGCTGGTCAGTAGCACTTTATACCTCTCTGTCCTCTCTTG
GTCTCCTGTCCTCCTTTTTCTTTGCTGGTTGTGTGTATCCTCAGGGGAACCCTAAGGATGGGGTTCATGGGGAAGAGAT
ATTATTTGAGGTCCTTTATGTCTGAAAATTTTCATTTTACCCTCACACTTTGTTGATGGTCTGGATGACTTTAGAATAT
TAGGTGGAACATAATTTTTGTTCACAATTTGGAGGGTTATTTTCTCTTGTAAGGATTTCAGATAAAGAGCCCAAAGCCA
TGTCTTTCTAGCTCCTTCCCCCACCCCCTTCACAGTTTTTAGAATTCCGGAGTGGTTTACTCTGGAATTACTCTGGTAT
TCTGAAGTTTGTTAATTGGGTTGCAAAGCCTTCAGTTTTGGGGAGTGTTTGTTTGCTTTTTAAATAATATCCTATGCCT
TATTTCTTCCTATGTGGGTTTTTTTTTTTCTTTCTTTCCTGAGATGGCTGTCGTGTGTCTGTTGATTCTCCTTAAGTTG
AACTTCATGCTTCTTGTCTTACCTTTATTGCTTTGTATTGAAACCAGTGGACAGAGAGTTCTCCTCAATTTTGTCTTAC
CAGCTTTCATTTTAAAAATGTTACTGATGTTTGTAAGTTTTAGAATCTCTTTTTTTTTCTGTATTTGATTGCATCTTGT
GAATGTTTTATCTTAACTTTTTTATTATTGTTGTTGTTGTTGTTGCCAGAATTAAACAGAATTCTTCTGCTTGTGGTTC
TGGCTGTCTGATCCTTGGCTTTGTGTAGTGAGATGCTAATATTGTTAGGAGGGAGAGGATATGCTCGTGTGCTGACCAT
CCTTGCTGTCAGCCAGAGTGCAGGTGGGCATTTCGCTGGAAGCCTGTCCCGAACACCAGTACAGAATTTTGTATCTAAT
TAGGGCTCTCTGCTCAGTGTCTGTAAAGAAGCCCCAGGAAACTTCCTGCCTCTGGAATGCCGGGTGATAAATCAGCTGC
CAGGCCTTGGGTTACCTGGTTGGGGAGGAATGGAAGTTTTCACCCTCAGGCTGTAGACTTTGACTTCTGGTTATAATTC
CCTGCCCTTATGACTCAATTTCCCTAGACCTGGGGTCTCCTACTGAACCTGGGGACCTGCTGCTGTGGATATTTGCACA
GTGGCTCCCTCCCTGTGCAGCCTGAGCTTCAGGGTGACCTTTGGTACCTCCCAGGTACCTCTAGGGCTGAGCCTCTTAA
GGGGCTTCTGATCGCTCCTCTACCTGCTTTCCATCATGCAGAAGTCTGATAAAGTAGATGAGTTTTTTCTTCCTTCTTG
GCTTGATAATTCAGTTCCCCTACCATCCAAAGGCTTTGGTCATTCTGAGCTGTGTCATTTGGTAGTTGTTTTGTAAGGG
CAGTGGTGGTCAGTAGTGGTGGACAGAGTGCTCATGATGTGTGGCAGAAGTTTCCCCGTGTCAGATACATAGTCCTCGA
AGGCGGGTGCTGACACTTCCACCCTGTACCCAAGCAGGCTGAAACAAGATTCCATCAACTGAGGCCCTTAGAAGCAACA
GAAATGTCTTTTTCACAGTTCTGGATGCTGGGAAAGCCAAAATCAAAGTGCTGATGGCTTGGTTTCTGGTCAGGGCCTG
TTTTCTGGTTCCTAGATGTATCCGTTCTCACACAGTGGGAGGGTGAAATGAGCTCTGTGGTCTTCATTATCAGGGCACT
GATCCTCCTTGTGTCTTTAAGACCTAATCCTCTCCCAAAACCCTGTTGTTTATCTATCTGCCCGCCATGGCGGTTAGAA
TTGTAACATGGGTTCTGGGGGAATACAGAGGTTTTTACCTTAGCACTGAGGAAGCAGACTTGGAGGGGGTCCTTGGTGT
GTGCCTGCTGCATGCAGTCGTCCTGTGGAAGTCCGCCTATGTTCTGAACCGTGTCTGTGTGGTTTCCCGTGTCACCAGG
CCCTGCTTCACTGCTCTCATTACTGTCCTTAGCAGACTACAGTACTGGCTACTGTGTCCATATGTGAAGGGTGGGATCT
TGATTCAGCGTGTTGTGGACATCGAGTAGAAGGCCAGGCCTTGACTCAGTTGTGCCTGAAAACTGGATCCCTGCTGTTT
CCAGCAGCCCTGGAGCCGCTGCAGTTAAGCCAGAACTCAAACAGTGCTGGTTTGTGGACCTGGTTACTGTCAGTGTCCT
CCTAGGTTCACTAACGTAGCCCTGTGCGCTGCAACTGCTGGAGCAGTGCCAGAGGTTTGGGAATAAGATGCCGGTGGCT
GGGAAGGCGTGGTGACAGTGATGGCCGTGATAGGGAGCTGTGGTCCATGGCGGTGAAGACAGCTTCGGAGAGAAGGAAG
CAGTTAAGCATTTCCTCTGGCTAGCTTGCTTCTCCTTTCAAACTAAATTGCTTCTGGGAAAGCCATGCTTGCTCACTCA
CAGCTTATTTAAAGACAGTTCACACTTGTTTAGGCACTTGCTTCTCAGAGCAGTGTAGGGAAGGAAAATTGAAGGATTA
TATATATGGAGATAATGACAAAAAGTGATTAGATTCCTTTCCTTAATGAAGTGTGTGTGTGTGTGCATGTGTGTATGAT
TGTCCCATGTAGGACTGTAGCTCTGGGATAAGATTATTGGAGGAGTGGTCACTTGATTGTGATTGATGGGGTGATAGGC
AGGGTTATTTTTCATTTTTCACTATGGTTGTTGGGAGGGTGTTTTTGTTTGTTTTTTGTTTTTGTTTTTATTTGTAGGA
ATGGTTACTTTTGAGAGAGGAAAGAGAGTCTTGGAGAGTTTGGGAAGGGATCCCAGGAAAGGAAACATTTACACCTCAT
CTCCAGGTTGAGCTAGACAGGCAGACAGAAGAGAGAAGCACTTAGGGCCAGGCAGAGAACATGGCATGGAGTGTGGGGG
CAGACACTGCAGAACTCTGACATGGCTGGGATGAGAGCCATGAGCCAAGGAGAGGAGACAGAGAAGACAGCTGGGAGAT
CAGCGGATTGGAGCTGTTCCCCTGGGAATGGAAAGCCACTGGAGTAGTTAGGCAGCACTCCAGAGGTGGAGGCAGGGGG
GATCAGGACTTCCTGGCTAGCTTTGTATATGTGAGACTCTGATTTACTCCCCTCCCCCAAACAAAATAACAACAAAACC
CAGCCATCAAAATACAGTACCCCACAACAAATAAAGCAGAGATAGGAAAGGAGTATGCAGTCGTAGTGGGTTGTAGAAA
AGATAGTGTGTGGCTCCTTGTCTATAGTTAAGACACCTGTCGGGGAACATGGGTTGGGGAGCAGGTGAAGAGGTGGGGT
GGATTTATCAGGAATGAGCAATGAGTTGGGAGGTTTACCCCTCTGAGACCTCAGGGCTAAGGAGAAGCTATTCTCTAGA
GTCCACTTTTTTAGATTTGGATGTCTGAGCCAAAGGACCCTGTCTCAGTTGAGATGAGCCAAGTGTTCTCCCCAGCTAA
GCTCCTCTCGTTCCTGCAGAGTCTCAGGCCAAACGCTTGGCTGGACAGGGGTGGTGTCCTCCCTGCTGGTGGCACTGGT
ATAACCTGCTAGGGGTGGCTTTGGCAGGAGTGGGTTAGCATCCTGAAGTGAACCCTTAACCCTAGGCGTTGGGTTGGGG
TTGGTGGTATTGGCACAGCTGAATCCGAGCCCATGTGTTCTTAATTGGGAGGCCGGGGCTGAGACATTGTGCAATTGTG
GGTTTGAGTTTCTTTCTCCCTGCACATATGGGGGAAAATTCTGTCCAATTATGGCTATACTTGTCCCATGTGAGTCACA
GCCTGAGTTAATGAGCGAGTTCTGAAGGTGCTGCCTCAGTGCTGCTCGGGTTTTAAGTCCACTCCAAGCGTAAGTGTGT
GTGTGTGGGGGGGGGGCACGTTTCTCAAACACTCAGAACTGAGAACCTCCCCAGCTTTGCTTTGGGGTTTTGATGCTGT
ACATCAACTGTCCTTTTAGATCTTGCCAGCCTGAGAGATAAGAAAAACCTGCCCTTGAGACCAAACTATTAAGTTACTC
TTAAACTACAGCAAAAGTACCGTGCTTTCTGTGTGTGTCATCCTTGTGGATGTATGTGTGTTCTAATCTGGATGTTACT
AAGCAACATTCCATTGTGTAGGGCCTCCTAAAACGTTATGTGCTAAAACTAATGTGTGTGTGTGTGGGGGGGGGGCTGA
GATGTAAACGCTTACTGAAGGGTCTGTTTTGCTGTCCCCAGCAATGACAATGCCGAGTGACTGTCATCCTTACATTTTA
AGTTGTAATAGAATGTGTATAGTTCTTAACTTGGGAGATGCCCATAATCTAGAGAGAACTAACACATCTTATGCACACC
AGCTATTTCACACATGTTTGTCAGCAGCATCTGGCACATTGTTTACTTTTTAAATAGATAAAACCAAGATTCTGGAAAA
ATACACAATTTGCTGGAGGTCTCAGATGCATGCAGTTACGAAGCTCTGAAGTGCACGCTTTCTACTCTGTTTCTGGGAA
GGGTAATAGTCTACCCGGCTCCCATTGAACTCACCCGGGGAGCTGCAACAACATCCAGAGTCCTGGCTATACCCTTACC
AAGTGGTGTAGTGATCTGAACTTCTGATCCTCCTGCCTTCCCCTCCCAAGCTCTGGAATTCCAAGTGTTTACAACTCCT
GCTTCACGTGGTGCTGGAGATCAAACCAGGGTTCCGTGAATGCCGTACCCCAGAGCCAGGTCCTCAGCCCTTGTAGTTG
ATCTGAAGTGATGAGTCAGCCCTTTGACTTCTGAATGCGTCACTGACTGTAAGATGCACAGCTTGTAAATTTGCAGCTG
ACTTAGTGGGAGTTCTAGTTAACAGACTGCTTTCCCCCATACCTCAGATTAAAGTGACATGCCCTGTTATCAAGGGTGA
CACTGTAATAACATCATCAGCCTAGCATTTCAAAAAGTCCAGGTGCTGTCCTGGGAGTGCCAGTGGTTTTCTCCTTCCT
GGTAAAGGGGGTCTCTATAGTAAGATTGCATACTTTGCTAGAAAGTCCTTAGGCCTGGGGCCTCAGAGAACTCCTGCTG
AAGTTCATGGGTATCTCTGAGTCTTTTGGATTTTTGAGACAGGGTTTTTCTGTTTAGCCCTGGCTGTCCTAGAATTTGC
TTTGTAGACCAGGCTGGCCTTGAACTCACAGGGATACACCTGCCTCTGCCTCCCAAGTGCTGTGATTAAAGTGTGTGCC
AGCACTGCCCAGCTGTATTCTAATTTCTTACAAGAGTTAAAACAGTTATGATGGGGTTGGAGAGATGGCTCAGCAGTTG
AGAGCACTGGTTTCTGCAGAGGACTGAGGTTTGACTCCTGGCAGCTGCATGACAGCTCACAACTGTCCATAACTAGATC
CAGGGGATCCAGTGCACTCTTCTGACCTTGGGTACCAGACACACATGTGATGCTCAGATAAGCCTGTGAGCAAAATACT
TATACACATAAAATAAAATAAACAGAAAATAAAATTTAAATAAAAAGTTGTCTTTAAGAATTCATTCTAGAGCTGGAAA
AATGGCTCTGAGGATAAATATGTATTGTATAAGGCTGGAGACCTCAGTTTGATCACCAGGACCCACCCGAGAGGAATGA
ATGACATCAACAAGTTGTCCTCACACCTGAGCCCTTCCCATAAACAAACAAACAAGTGAATAAATGTATGTATGCAATT
AAAAAGTTCACTCTGCTATAAGCTTTTATGGGCTTTGACAAGTGCATAATGTCCTCTGTGTACTTCATCATATAGTATA
TAAAGAATATATCAGATATACAGAGTAGTTTTAATAAAAAAAATCATAGGAGGAGGCATCTGCATTTATAATAACAACA
AAAACCCAACCAGTCAACCAAACAAAAAGCCAAGAGAGCCGGGAAAGAAAGTTTCCAGTATTCATTTTTCTGAAGTCCT
GCTGTTCCTCTAACCCAGTGCACTTTTTCTTATCTCTTACATTGTAGTTCTCATTTCAAGAAATTAGACTTTATAGTCT
TTTTTTTACTATCTGCATTACTTATTCTTTGTTTCTGCGGACATTTTTGGCCACGTGGAATACGGTTACATAGTTATTG
CTAGCACACCAGTGCCTGTTGGTGAGTTTTAGTTGTTATCTAGTGCTCTGTACATTCTTTCCTTTAAAAAAAATATGTG
TGTCTATAAATATATGTTTGGATGGCAGATACTGTGAACATTTCTGGGTGCTGAGTGTGTGTGTGTATGTGTGTGTGTG
TGTGTGTGTGTGTGTGTGTGTGTGTGTTGTGTGTTTTGTGTGTGTGTGTGTGTGAAATTTTTTGAGACAGGATCTATTG
CTTGCCCTAGAATTCAATGATGAGGCTCAGCTGGGTGGCCGTCCAGGAAGCTCCAGGGAACCTCCTGTCCCCATCTCCT
CAGGGCTGGAATTACAAGTGTATGCCATTGCACCCAGCTTTTTTGTGGGTGCTGGGATCTAAACTCTGGCTCTCTCTTG
TAGCAAGCATGTTAGGAGCTGAGCACCTCCTCAGCCTGCCTACCTGCCTTTCTTTCTTTGCCTTCTTTTCTGTTTTTGC
TTTTGTTTGTTGGTTTCTTTGTTTTAGACAGACACTTGTTCTGAAATTCAGGCTAGCCTCCTGCTTCATTCTTCCCACA
TGCTGGAATTGTAGGAGTGTACCAGGATGCATGACAGCTGAACATTTATGAATTCTTTTAAATATCCTTGGATTTTGTT
GTGGGATGCAGTTAAATGACTTGGAAACAGTTTGAGCCTTCTGGGTGTTACTTATAAGGCTTGTTGTCAGGCTCATTGT
TCCCCACAGGTGAGTTAAGACCCTTCTGCACTTATCCCACTAAACCGTGAAGGGTGAGCCTCCGGTCTGGCTGGTGGGA
GGATCCAGATTCTGAGTATTGGGCACTGTTCCCTTTTACCCTCTCTCTGCCCCCTTCTGGCTTCCACATTTGCATGTCG
AGTCACAACTCAGATGGGCTCTGGAGCTCAACACTCTGGACCTTGGACTTTCCACTTTGTTTAGCTCTCTGTGCACCAT
ACCTCTAGAATCTTTTTCTAGATTCGCACATGCCACAGCATTCATGGGGAGGTCAAAGGACAGCTTTGAGGAGCCAGTT
CTCTCTGCGCTGTATTGAGCTTTGGGCATCAGGCTCAGTCCTGAGACTCTGTGGTGAGCGGTCACACCTGCTGAGCCAT
CTCTCTTGACTTCAGCATCCTTCTCTGCACTGTAACTGTAGCACTTACTACTTGTGGTCTTTGTTCTAGGGATGGGTCA
CTGTTGCCTCCTGATGAGTCCTTTAAATTAATGAATCAGTTGAGTCCAGTCTTGATTATATCAGATGAGAGAATCAGTC
AATCTTCCAATATTCTCATCAAATGCTCGTTGTTCATGTCTTCCCTAAGGTTAATTCCTTAATGATCAATTAGAGGAAA
AAGTAGCCCTGCTGAAAGAAGCAGTTGTGTAAGGCTTGGATCTGTTTGTGCAAGCACGCTTGTGAGCATATGGTAGGTG
GCAAGTGTGGGAGAGTGTGGGAGCCCATATGTTTGCATGTTAGTCGCATCCCTTCCTTCCACTTTAAGTGGTTAGGCAT
GTGTAGCGGCAAATCTTGATGGTCAGGTTAACACACCTGGAAAGAGGGACCCTCAATTCAAGAATTGCTTCCATCAGCT
CGGCCTGTGGACAAATTTAGGGGGGCATTTTCTTGATTAGTTACTGATGTGGGAAGGCCTACCACACTGTGTTCTGTGT
CACCTCTGGGCATGTGGGTCTGGGCTGCATAGGAAAACTATCTGAGCTGGGTAGTGGTGGCACACACCTTTATCCCAGC
GCTCAGGAGGTAGTGGCAGATGAATCTCTGAGTTCAAGGACAGACTGGTCTACAAAGTGAATTCCAGGACATCCAGGAC
TACAGAGAGAAACCTTGTCTCAAAGTACCCCTCCCTGCCAAAAAAAGTTATTTAAGCATGAGCTAGTGAGTGGGCCAGT
ACGTGTAAGAGAGTTTCTCTGTTTCTCAGTGGGTTCTGCTTCATTTCCTGCCTGGACTCCCTCATTGATTGGCTGTGAC
CTGCCGTCTTCTTTCCCGAGTTGCTTTGGGTTATAGTTGTTTATCAGACCTACAGAAAAGCAAGCTGTAACTAATGATC
TGCCTATGTAAGATGAGTTGCTCTTCGCACATGTGCTATAATGCATTTGAATATGCTGAATGAGCCTCACAACAGCCTA
TGTTAGTCACTGCTGGCGATTTCTGTATCTTTGCATTAAAAACAAATACCCTAAACATTTTGAGCTGTTTCATTTCTGG
TTGGGCTCTGCTGAGGAAGGTAGAATGGCTCATCTGTTTGGTTCTGAGGAAGGACTGGGAGTAAGCTGCAGGACACAGG
GAAGGAGCCTGCTGATCCTCCCCAGGAAGAGCCAGATACCTGTAGCCCCGAGCCCCTTTCCTCACATTTAACTGTGTGA
GGAAACACAATTCCTGGCCTAAGACAATAGGCCACTTAAGCACAACGCACTGGAGGCCTCAAGTCACCGGGTCATTTAG
ATATTTGGTTGGTTGCCTTTCGTAACAGTATTCTTTTACAAGAGTAATATGAGCTTATTTTAGGAGCATGGGGTATCTT
TGGTGACTAAAACAGATAAAGGACCTGGCCTTGTGGAACTAACTTTTGACAGAGACCTATGTGATTTGCCTTTGAAATT
AGCTTTGGGATTGGATTCCTGAGATAGTAGCACCCTGCTGCCCGTCACTGGCAGGGAGAAGAAGCGATGCTTGTGAGTT
CTCTGTGTTTTATGATAACAGCCTACATTATTACCAGGCAAGTCTGAGTCAGTCCAGCCTGAGGTAAAGTGTGCTCCTG
TTAGCAGTGGAGGTCAAAGAAGTTGGGCAGTGCTGCCATGAAGCCCTGGGTATGTGGCTCCATTTCTGTGGCTGTGTCA
GTGGTACAGGTATAAACTGGTCATATCACTTTCTTCTTCTAGGTCAGGGAACACACCAAGCTATTGTCTAGAGTCTCTG
TCTTACCAGATGCTGGGGATTAGGGCTCTGTGGCGGGGAAGTGGTGTTTTATGTTTTAATGCTGAAGCTTGTGTATCCT
TGAGAGGGAGTTGGGAGGTGCCTTCTACACTATTGGCTATTGTCCAGATCATATGCAATCACAGTTTTTAGGGGAATCT
TCTTACGCATTTCTCTTTGACCTATATTTTCCTTGGGAGTGAAGTGTTTGGTTGTTTTAATTTAAAGTGTGTTTGCTAC
ATTTGCTATAGTGTTTGCTGAGGTGGGGAAGGTGACAGCACATCTATTTGAAACCACGCCTGCATAACTTCTTCCAGAG
CACTAGCAGAGACAAACCAGAGCAGCACAGCTTTTGTGATGGATGGCTTGAGTGTCTGTGATCAAGCATCAGAGAATTA
CTATGGGTTTTGAGAAGACAGGTCTTGGTCTGAAGGAGGGAGCCTAAAATACAAGGAGCAGAGTGGGGGAGTAATGGCC
TTTTGTTGGTCAGAGCTGTCTTTCTCAGCACCCATGCTTGTCTTGGTTTTTCTGTATGTAACTATGCATTGTTACCTCA
GCTCTGCCAAGATGTTTGAGATAGAGACAGAGACAGAGACAGAGACAGAGAGAGAGAGAGACGCATGAAGCAAACACTG
TGTGGTGGAGTGCCTGAGAGTTGTTCCCAGTGTTCCTGCTCCAGTAGCCACAAGGCTTGAAGAGACCTTTGTAGTGGTG
GAAGAAGGCGTTCCTTTCCTGCCATCAAATGTCGCAGTTCTCTGCTGAGGGCAGCCACGTTCTTCTGGGACCTAAACTC
AGAAGCTCTGCAAAAGCAGCCTCTGCAGCTCTGGCAGTCGCTGCTATGTGAAGTTAAATGTCGCTTCTCAATAAATTGT
TTGGGTGTGCACGGGCATAGCTAGATTTCGATGAACAAACTTATTTCAGTTTATTATTTTATATAAATTAGAAACATTA
GAAGTACGAGCTTTTTATTTTGTATCAGTAAGTGTACTTAGTGGTTATACATTGCTTACAAATAAACAAAAAAATAAAT
AAATTCCAAGAAAAAAACCACATTGATTACCTTTTTCTGATTAGACCTAGAAGATTGTCTTGACTTGAAATTTTTTAAA
AGTTTTGATTGGTACGTGGGAGCCATGGATGTATCATAGAATACTTATGTTTTGGGTGACATTTGGAAACTACTGCTGA
TGTCATAAAATTATAGAATTTTTAGTGGAGGAGGGGATTAACATCATCTGGTCTACCTCTTTTGTCTTATAGTTTTGAA
AGCTGACTTATGAAGATGGCTTAGGAAGTGAGTGCCAAAGGCCTCTCACCTACTTAGTCTCAGCCAGGAAGAGAGGTCT
GCACGGTGAAAGCTGCTCTGTTTTCTTATTTTAACTGATGCCTGACAGGTTGTAAAATAACTTTTTTTTATCAACAGCC
AGCTATTCATGTGTCTGCCACTGAATGCTACAATGCTACCCTAGGTAGAGGAGACTGCTGGTCCTGTGAGCAGCTCTCA
GACATATGTCCTGTCAAGTGCACTCTTAAAAGGAGGCTCTTCAGTGACATGCAACTTACCCACAGGATGGCAGCATTGG
AATCCGTAGTTAACTTTCAAAGTCATTTGGAAAAATTTTAATTAGTGGATTATTAATTTGGGACTTAAAATTGTATAGT
GAAGAACTTATTTTTCTTGATAGGGTCATGAGAATTTTGCAAGAGTAACAAATTTTATATCATCAAATATGGGTTCTTT
AACATCAAATTATGAACAAATGTTATCCTTTCTTAGAGAGAGACACACGCTACTTCCCTACTTTGCTGGAATTGGCAGC
AAGAAGCTTAAGTCGAGCTGATGAAATTGAGCAGCTGAAGTGCTTATCCTGAGTCAGAATGGCAGTATTCTGTGTGTTC
TGATGACAAAATGCAACCTAGTCAGTCCATGGCCTGTGGCCTTTGCCCTCTCTCCCTCCTCATAGCCCTTTCCACTCTC
TGCCTGTTCTCCTTTCCCTGTTTTGTTCCCTTCTCTTTGGCGCTCCCTCTTTATATGGTAGCGTTTCAGTTCTTGAGTC
TCCATGGTGCTGCTTCTTTCAAAAATCAGTCTGTCTGTCTGTCTTATCATCTATCCATCTATCATCCATCTATATCTTC
TATCTATATCATCCATCTATATCTGTCTATTATCTATCATCCATCTCTCTCTATCTAATCAATCAATCAATCTATCTAT
CTATCTATCTATCTATCTATCTATCATCTATCTGTTTATCATCCATCCATGTCATCCATCTACCTACCTACATACATAC
CTACCTATTACCTATCTCTCTAGGGTGTGTATGTGTGTGTGTGTGTGTGTGCGCGCTATGCAAACAGAAGGTTTGAGGA
CAGCCTGCAGGAATGGGTTTCTCCCTGTTTGGCTTCTGGGGTCATCTTTGGTGGCAGGCACCTTTACCTGTTGAACATC
TCACTGACCTGTTCTGTATCTTTACAAGTGGATTCACACACTATGGGATATTTTTTCTGACCATCTTCATGTAGCACAA
TGTTATGGAGGTTCCCATACATCTTTTCATTCTATTTTCATACCATAATTTACTTTTTATCTGTTCAGGGACCTTCAAG
TTACTTACATATTTTGGCTGTTATGAATAATGGTGCTGAGAATATGCAAGTACCCGACTGGATACATGTTCAAGTTAGG
TACATAGAATAGAATTGAGGGTTATCTGGTAACTGTCTTAACTTTTTAAGGCACTGCTAGGGGCATTTTCAGTGGCTGA
CTGTTTTGTATTCCTACCAAGACTGCAGGGGTCTTGTTTCTCTGCACGCCAGCTCTTGCTATTTTCTGCTTCTTTGGTT
GTGGTTATTCTAGGCAGTATGAAGTACCTCATTTTTATTTTGATTCTGTTTTCCTGGTATCGAATGATGTGGAGCATCT
TTACATGTAGTTGTTTATATGTCTGTCTTCTTTGGAGAAATGGGTGTTCAAGTCTGTTGTCCACTTTTAGATTGGACTG
GTGGTGGTTGCTGAATTGTAAGAATTGTTTATATATCCAGGATTCACATACTTGCCAGGTGTGATTTGTGGGGCCATCC
TCCAGTCTGTGTGTTCTTGTCTTTGTAATAGTAGGTTGTTTCGTTGTTTGTTTGTTTGTTTGTTTGTTTGTTTGTTTCT
GGTTTCTGCATAGAATTAAAACTTTTTTAAATATGAATTTTGGCTTATCTACTCTTTCTTTCCTTCTTTCTTTTTTGTT
TTGTTTTGTTTGAGACAGGGTATCTTTATGTATCCGTGGCTGTCCTGGAACTGGTTCTGTAGACCAGGCTGGCCTTGAA
CTCAGAGATCCACCTTCCTCTGCCTCCCTAGAACTGGGACTGAGGGCGTGTGCCACCATCACCTGGCTAGTTTATCTTC
CTTTTCCATCCCTTGCTTGGGCTTTTGCTATCCTATTGTAAGAACTCGCCACCAAGGTTATGAAGATATATCTTTAATA
TTTTTCCAAGAATTTTATGGGTTTTTTGTTTTGTTTTTCTTTTGTTTTTCAAGACAGGGTTTCTCTGTATAGCCCTGGC
TGTCCTGGAACTCACTTGGTAGGCCAGGCTGGCCTGGTTTTATGGTTTTGTCTTATGTTTGGGACATCTATTTTAAACT
AATCTTTGTCTGTGGTGTGAGGTAGGGACCCAGTTACATTCTTCTTATAGAATAACCATTGTCCTACCATTGTTTGTTG
ATGGTGGTTTTGGTACCCTTATCAAAACTTGAGTCTTCAGTTTTGGGTGGTGGGGGTGGGAGGTGGCTTTAAGACAGAG
TATTTCTGTATATTCTTGACTATCCTGGAACTCACTCTGTAGACCAGAGCTGGGCTCAAACTCACAGAGACCTGCCTGC
TTTTGCCTCTGGAGTTCTGGGATTAAAGTTGTGTGCTAACTACCACTGCCCACTGCCTGGCAAGTCTTCAGAATTTTAT
AACTTTATTTCTGGCATCAATTCTGTTCTGTTAGTTTACACAGCTGCCATTATGTTAGAATCATACTTTTTAAATTTTT
CTGAGATTATAATCATATCATTTCTCCCTTTCCCTCAACATTCCCATATATCCCTCATTCCTTGTTCAAATTTATGGTC
TCTTGGTTTCTTTCTTTTTTGTTTGTTTGTTTGTTTTGTTTCATTTTTAATGAGAGAGAGAGAGCGCCTAAATACATAA
ATACAACCTGCTTCGACTGTGATGTTATTTATCTGTGCTTCCAGGCCTCATGAGCTTTCCCTTGTCTACGTTAGATGTC
TGTTGTTGTTCTTGTTCAGTTCGTGTTTAAGCAGTCATGTTGGTGAGACTTTATGGCTGTCACTTATGACATTCTCACG
GCAAACTTCCTGTTCCTCTGATTCTTAGAATCTTTCCTTTTCCTCTTCCACAATGATCCCTGAGCCATAGTTACAGGAG
TTATGTTGTAGATGTATCTGTTGGGATCGGGCGCCATAACTCTTCATTTTGCTTGGTTGTGGTTTGCTATAATGGTCTC
CTTTCATTGTAAAACGGAGAAGTTTCCTTGCAGAGGGGGAGGACTTACCTGTGGGTGTGGGGAAATATTTAGGATGTGG
TTAGGGGTCATGATGATTTAGTAAAGTAGAAGACCCATGATTTTACTAGCTGTGGGTGGTTGGCTGGGTTTCCAGGACC
AGGCATGATTTCCCTCTTGTTGAGCAGGTCTTAAGTCCAATTAGAGAGTGCTGGTTACCGCCGAGGTACGTGTGCCACT
TCTGCACTGTCTTGATTTGCATAGCTTTGTAGTGAGTTTTGAAACTGTGAAATGCGAATCTTCTTTTATGTGCTTTTAA
AAGATTGTTTTGGCTATTTGGAGCAACTTTGCAGTTTCATATCCACCTATCTACTTTTCTATTTCTGTGTAAAAGGCAG
GTGGAATTTTGGTAGAGAATGCACTGACATTGTACATTTGGGTTATATTGCCATTTGACTAACCCTGCTGTCTTCCCAT
CCGTAAGCATGCGATGTTTTTCTGTTTATTCGGTTGTTTCAGTACTTTGTGTTTTTTAACTTAGATTTTCATTTCTTTG
GTTACATTGCTTCCTAAGTATTTTAGTCTTTGCGATGCAGCTATGAATGGAATTGGTTTCTTAATTTCCCTTTGGAATT
GTTCTTTGAGGAACTGACTCTTATGCTTTCTATCTTTCCATCTGTTGTTTCGCCTCAAGATGGTGACTCCAAACCCTCC
TCTCTTTTGTTGTAAGGTCTTTACTGTATACCTTTACCTTATACACACTAGTATTGGGAAAAAAGACCCCAAAAAAGTG
ATTCCCAAAGACTTTATTCTCTAAATCGAAATTTCTTTTTTACCTTCCTCTATTCCCCAAAATGTCAGAACCTGTTCTG
ATTTTCTTTTAGCTGTTCCTCTTTGGACCATGATGGTCCCAGTTTCCAGGAGACGTTTCTCAAGGCGCCTGCTGGGGTA
ACTAACCCTGAGGCTCACCGAGCCTTCCTCCCACCCCCTTTCCTGGTCCCCGATTTTCTAACATTCCTCTGCTCTGGCT
TGCTGTGAGACTGGCTCATTGCTGCGTTGTGGAATGTGATCCTTTCCTCAGCACCACTCCTCAGTGTCCATTTAACCTG
GTCCATGTTTGCCCACCATTCCCCATCATCATAACCTCTGCCAGCCCCTGTTTCTGAGCTCAGTGTGCATGTGATCACA
GTGGTTACAGATGATGCATGGATAACTGGTGATAGTTGCAAGGGGCAGCCTTTACTGTCTTCACTTGCCTGGTTTTCAC
TTTGAGTGAAAAAGAAAGGAGATTAAATCCAGAAATATAAACTATGATACTCCTTGTAAATTTAGTGTTTGTCAAAAAA
AAAATCAAGATAAAATTGTTAAAAAAATTTTTATTTCCTTTATTTTATTTCATGGATGCCAATGCAAAACATAGACTTT
ATTGATCATTTAGAAAATATATGTTCATATCATAAAGAAGAAGAAAAACATTAAACACTCATTATTAACTTTTGGTAAA
CAATCCTCCTAGCCTATTTTTGCATTCTTGTTTTTCTTATAAAAGGTGAAGAAAGCAGGCAGATTGTACTTCGTAACCT
ACATGTGTTTCCTGTCTCTGTGCCTTCTTCTCACCTACTGCTGACCAAATTTTGGCCTGGACATTTTCTGCCTTAAAAA
GTTGGTTTCACATTTCCTTACAACCGATAACTTTTAAAATAAATAATAATATTATTTATGTGTATGGGTAGGAATGTGG
TTGGTATATATGTGGCCGTCAGAGGACAGCCTTGAGGGGCTTCTTCCACTCTGGGATCTGAGCATTAGATTTGGCTGAC
CAATGCTTGTCTAGCAAGTCCCTTTACCTACAAGGCTAGCTTATTAGCCCAGTGTTTCGTTTTTGTTTGAGAACACTAT
TTTAACATAGTCCAGGCTAGCTGAAAATTCAGTAAGTAACTGAGATGCCTTGAACTCAGCAGCCTGTTACTAGGGTGTG
ACCAACCTGGTTGCTGATAGCTTTATATAACTGTCGGGAGGGTGGTTTGAGAGGAGACAGTTTCTATAGTGTATCTGTC
ATCTTGATGCGTGTCTTACTTATGATTATACTAATTATGGATATTCATGTCTTCTATTCTCTGAATCCTAATTTCACAC
CCTAATTGACATTCTTTATGTCTCTATGTTTAGAAATGCTTTACATTAAAAATGATTTATTTATTTTGATTGATGTGTA
TGATTGTCTGCATGAATATGTGCTTCATGTGAGTGTGGTGCCTATAAAGACCAGAAGAGGGCATCAGATCGCCTTGAAC
AGTAATTGCAGACAGTTGTGGATGCTGGGAATCAAACCTAGGTCCTCTGCAAGAGCAGCCAGTGGACTTAACCACTGAG
CCATCTCTCGAGCCTCTGAAACACTTTATGCTTTGATTTAATCAGCAAAGATTGACAGTTATTTTATCATCGTAAGTTT
CTCCTTGGTATAAAGGATTCTTTGTTCCCTGTTAAAAACAAAAAAGTTATTCTTTCTTGGTATGATTTAGAGTTACTTA
CTGGGGTATAAATATGTAATTGGCTGTTTTCTGAGGCCTCTGAGAATATGCTTTCTTTGCATAGGATCAGAATTTGTCT
GGAAATGGACTTTCTTGCTGGCCTTTTCTTCCCTGGATGCTTTGTTCCAGCATCCTAGAGTTTTCCATGCCAGTCTCCA
CAGCTAGTGAACACGGCCAGTCTCCACAGCTAGTGAACACGGCCAGTCTCCACAGCTAGTGAACAAGGCCAGTCTCCAC
AGCTAGTGAACAAGGCCAGTCTCCACAGCTAGTGAACACTGCCAGTCTCCACAGCTAGTGAACAAGGCCAGTCTCCACA
GCTAGTGAACACTGCCAGTCTCCACAGCTAGTGAACAAGGCCAGTCTCCACAGCTAGTGAACAAGGCCAGTCTCCACAG
CTAGTGAACACGGCCAGTCTCCACAGCTAGTGAACACTGCCAGTCTCCACAGCTAGTGAACAAGGCCAGTCTCCACAGC
TAGTGAACACTGCCAGTCTCCACAGCTAGTGAACAAGGCCAGTCTCCACAGCTAGTGAACAAGGCCAGTCTCCACAGCT
AGTGAACACGGCCAGTCTCCACAGCTAGTGAACACGGCCAGTCTCCACAGCTAGTGAACACGGCCAGTCTCCACAGCTA
GTGAACAAGGCCAGTCTCCACAGCTAGTGAACAAGGCCAGTCTCCACAGCTAGTGAACACGGCCAGTCTCCACAGCTAG
TGAACACGGCCAGTCTCCACAGCTAGTGAACAAGGCCAGTCTCCACAGCTAGTGAACAAGGCCAGTCTCCACAGCTAGT
GAACAAGGCCAGTCTCCACAGCTAGTGAACACGGCCAGTCTCCACAGCTAGTGAACACGGCCAGTCTGACATGTGTCTT
CCCATCAGTCCTCTGTCAATGCTGTAGGTGGAGATTCTTCCTAAACTTTGGTCTTTCTTTTTAGTACTTTAACAGATTT
TGCCTTTTGCTATATCTTGGTATTTCAAGGGATGGTGGAGGAAGGATTTTTAGTTATTAATGATTTGTCATTACTAGTG
TCAACTTTAATATACCAAAGATGACATCTTCTCAGTAATTTTTTAATTGAGAAATAATTGGGATGGGTGAGCATGTACA
GCCTTTTTTCCTAGCTCTTACTAACTTTAATGGGCTTTTAATAGAAAGTATCTTAGTTGCTTTGTTGTTGTTGTGTGGA
GATACTTGACCAGGGCAACTTAGAAAAGAAAGCGTTTAATTGAGGACTTGCTTCAGTTTCAGAGGGTGATCCATGGTCA
TCACGGCAGGGAGCAAGGCAGCTGGCAAGTGGGCACGGCACTGGCGCAGTAGCTGAGAGCTTACATCTCCCACACCCAC
AAGGCCCATCGAGAACGAATTACCTCAAAGCCAGTGACACACCTCCTCTAACAAAGCCATACCTCCCGACCCTTACTAA
ACAGCTCCATCAACTGTGGACCAAACATTCAAATGAGCCAGTAAGGGCCATTCTCATTTAAACTGCCACAGAAAGTAAT
TGAATTTGGTTTTAGTGACTTTCAAAATGCTGTATGCATATTTAAATGCTTTTTTAAAACCTTTAATTTTTTTTCACTT
ACTCTTCATAGAATATTTAAACATTGTTGTTGTTGTTGTTGTTGTTGTTGTTGTTGTTGTTGTTGTTAGATGAAGTCTT
AGTGTATTGTCTTGGATGGCCTGGCCTGGAACTCACTGTGTAGTATCTACCTGCCTCTGCCTTTGCCTCTGCCTTCTAA
ATGCTGGGGTTCAAGGCATGTACCACCTTGCCCACCCTTCATAGAAAACTTAAGGAGAAAGAAAAGAATACAGGGTACA
TAAAAATCAAGTATAACCAATCTATTATTCACTTCTGTCTTAGGGTTTTATTGCTGAGAAGAGACACCGTGACCAAGGC
AACTCATAAAGAACAACAGCTCACTGGGGCTGGGGCTGGCTGACCGTTTCAGAGGTTCAGTCCATTATCACCACGGTGG
GAAGCACGCAGTGTCAGGCACACATGGGGCTGGAGAAGGAGCTGAGAGTTTGACATCTTGATCTGCAGGCAGCAGAAGG
AGACTGGGCAGAGCTTGCACAGATATATGACCTCAAAGCCCACCCCCACAGTGACACACTTCCTCTATCAAGGCCACAC
CTCCTAATTATGCCATTCCTCATGGGCCAGGCATTCAAACATGTGAGTCTATGGGGGCCAAGCCTATTCAAACCACCAC
ACTACTTAGTATATTTTGGTATTCACTTTCTTTTTTAAAAATGGAACATATATAATGTGCACATTCATATATGCATATC
CATGTCTGACTTTAATTTCTTAGATATGTATTGAACACTTTGAGGTGCCAGGCAGTTTTAAAATTTTGCTGTATATAGT
TTACATCTTGCCTACTTTCACTTAGTGTTAAGGACTGGACAAAACTGGTTCACCAAACTGTGTTTGTAGAAGCCCCGCT
CACAGTGTCATAACTCTTTTCAGGGTTTTTTAGTTCAATCCCTCCTTCCCCTCCCTTTCCCTCCCTCCCTCCCTCCCTC
CCTCCCTCCCTCCCTCCCTCCTTTTCCCTCCCTCCCTCCCTTCCACCCTTCCACCCTTCCTCCCTTCCTCCCTTCCTTT
CTCTTTCCTTCTTTTTGTCATCAGAGTTATTTTTGTGTTGCTGGGACCAAAAAACCTGATGGAAGCAAGCCGAGGAAGG
ATAGACTTGTTTTGTTCCATTGTAGAGGGTCCAGTCTGTCAGGACAGCAGGAGTTCAGGCAGTGGCCTCTTCACATGGC
AACAGACAAGAAGCCGAAAAGGCAGGCTGCAGCCAGTGCAGACACAATCTTCAGAGGCCTGCTCTTAGTGACTTACTTC
TGGCAGGCAGGGTCTGCTCCCTGAAAGCTTCTTCCACATCTTTTGAATCAAGACACAAGCTGGGACTGGGCACGAACTC
ACAGAGACCCACCTGCCTCTCTGCCTCCTGAGTGTTGGGATTAAAGGCATACACCATCACTGCCTGGCATGACTGTTGA
TTATTAACCACTGCTGTCTTGCTAGGAATTTATATTACTCACAAGTTTAGAGTCACAAATTGCTGCCGCTCCCTGCTCA
GCATTCAGAGTTATATTTAAAGAACATAAGCATTTCTGTTATAATATCAAATCCACTCCTAAGAAAACTTTCAAGAGTG
CTTATTCACTAAAAATAACAGGATTATGGTGAAAAGAAGGTTCAAGGCAGACCATTCAAGACTTCTACTATTTCAGACC
AGAGAGAGCTAACAATAGCATAATTGGTACCTTATGAGGTAAGGTGGGTCGCCTAGTCAAACCACTTATTGTGGCTGAG
GCTCCCTGAGAGCAGAGTAAAGCTATTAAAGTGTAAGAGGATGGAAATTGCAGGCCTCGTGGTTGTGAGCTGTGGGGTT
GGCCTGAGAGTGGTACAGGGGGAGTGCAGCTTGCTCTGCCAGGACACATGCACAGGCCAGACAGTGCTCCTCTGGGGAT
AGAGCCCTTGGGCAGTGCTCACCATTAATATTCTTGAAATGGAGCTGAAAGGGCTTGCGTACACATGCCTGCCTCGAGG
CTGGGGAGGTGGCTCAGTTGTTAAGAACACATAAAACCCTTGGAGAGGACCCGAGTTCAGTTCTCAGCACCCATATCAG
GAGGCTAACAACTGCCTGTAACTCTAGCTCCAGGGGATCCAATACCCTCTCCTGGATTCTGCAAGCACTTGCACTCACA
TGTGTACACACCCTTCTGCCTCCAGTTAAAAAGAAAACAAATTCTTTATATACGGCTTTATGACCTTGACTCAAATCAA
GATATTGAACATTGCCATCTTTGCAGGAAGTCCCCTGTACTCCCATCTCCCATCCTTCTTTCTAGATGAGCACAGTTCA
GAGTTTTATCCCAATTAGTGCTATATCTTCTAGAAGTTTTATCTAAATAAAGTCTAATAATAGTTTGCTATTCTCATGT
GTCACGTTTCTTTTATTCTGAAAAGTCCATCCTTGTTCCAGAGTCTGGTTTTGATTGTAGAGTAGTAGTCCATTGTTGA
ATGTAAACAATTGGTATCAAGTTCAGTGTTTGCTGAGGAACATTTGTGTTGCTTCCACTTCTGCTTATTAGTAGTAAAA
GGCCTGTGAACATCTGCACAAGTGTTTTGTGGATACAGTGTTTTCATTTCCTTTGCAGAATTACCTCAAGAGGAATTGC
TGGCCATAGAGTAGGTGTGTGTTTAATTTATAAGCAACTGCTAGCTTTTCAAAGCAGTTGAGCTGTTTTTCACCCTGGC
AGGGATGTTCTGAGCTGACTTGCCAGCATCAATTGTAGTTATTCTCTTGGGTGCTAGTGGTTTTCATCGTACTTCTAAC
CTTTGTCTGTCTGAAGATTAATGGTGTGTTTTAGCGCCTCTTCACATTTTCATGGCCCCGTGTGTGTCTTACTACGAAG
CGCACCAATCTCCCTCCCTTCTACTCATAGAATGTGGTTGAGAGCAAAGTTTACTTAGGTCTTGAGAAGAGGCCTGGGC
CTCTTCAGGCCCATTCCTTGTGGTTTCTCTGCACAGCCCTATCTAAAACATACAGTTTCTTGGAAGAAGGCTAACCAGC
ATTTAAAGGGGCAACTGGCTTTTCCCCATAGGTTTTCTCCTAAAAGCACTTGTCTAGCACAGCCTTTCCACTACTTGAT
TTCCAGTGTGTTTAACAGTTCCACAGCTATAATGTATTACTATTATAAGTGTGAAATTGAGTGGGAGTAGGGACATCCT
TACCTTGTTAATAATTTTAGAAATGAGAATGTGAAAATTCACCCTTGCGCATGCGAGCTGTATGGCTTTGCAAGTGCCC
TTTGGTAAAGACAGAGAAAGTGTCTGTCCGTTTACTATTTGTTGTCTGTTTTTATCCGTATAGATTTTCAATCTGATAT
GCCATCACTGTTTCGTTTCAAAACAGGGTCTCGTGTTGCCCTGGTTGGCCTCAACCTTGTTAAATAGCTGAGGATGACT
TTGAACCCCTGAACTTGCAGCACTTTGCCTGTGCTGGAGTCACTGGTGTGAGTCACCACACCCGCCTTCGTTGTTTTCC
CTTTTCTACTAATGTGGTGACTCACTGACTTCTTAGCGGTCACTTGGTTATGCTGCGCTTCCCCTTTTTCATGCTTTTC
GTGGCTTATGATTGTCCGTACGTCTGCCCGTTGGGTATGGGGCTGTTAGTCTGTGGCTGGGGCTCTTGGCTTTTGCTTT
CTGTGTAGGAAGCTTTGCTTGGTACAGTGCTGCATCTTTTGTGTGTAGAGGATCTCAGTTGTAAGTTTTGTTACCTGAG
TCGTTTCACCTTGAGCTATAGTTGTGTGTTGTCATTCTGCCTCATGAGTCTTTTTCTTTTTGTTACTTGACATATTTCT
GAACTTTGTTTTAATCAATCTCTCTAGACTAACTATCTCAGGCTTGAGTGAATTTTTGTAAAAGGTTTATTATTTTTAT
GTGTATAAGTGTTTTGCCTGCGTGTGTGTGTGTGTGTGTGTGTGTGTGTGTGTGTGTGCGCGCACCTCATGTGTGCAGT
GCCCACAGAAGTCAGGGAGGGTTTAATCTCTGGGACTGTAATGAATTGTTGGGAGGTGCCATGTGGGTGCTGGGAATCA
AACTCAGGCCCTTGGCAGAGAAGTCAGTACTCTTAGTTGCCACGCCATCTCTCCAGCCCCTTAAATGAATTTAAACTGT
ATTATTGTGATTACTGATGTTTTCTTTGAATTCATTTTCCTTTTCCATTCAATCTTTCCATCTTCCTGCCTCCTCTACA
TCTTTCTCTAACAACACCAGCCCCCCCCCCCCCCATGTTCCTTAGCATTTTCTGCCTTCAGCAGGCAGAGCCTTCTGCA
CAGGCTTCCAGCTCGCCCATGTTGTATCTGGCACCCTGCGCATGATGCATAAAACAGTGCTACACAGATCTCGTTTTTT
GACTTATCATATCTGGGTCCGAAATGAAGGAGCTTTGACAGATAAGCTGTCTGTGGCAGAGCTGTTGGTCTCTCTCTGT
GTACTGAGGGGTCTGAGGTATCCAGGTGACCTCCTTCATGGATGTGCCATTTAAGTTTCTGTTTGTCCTGTCTGAGCAC
TAGGGACATCTGGACTAAACTTTAACAGTAACCATGTTTAAAGGGAGTTAACAGGCCCCTGTTCTCTCCGTTCTTTGGC
TCTTTGCCTGTCTCTAACACTTGCTGATAATACCTCCTTCCCCCTTTTGTCTCAGTGCCCAGGACTCAAGGTCACTGAC
TTGGTCGCATCAGTCATTAGCTTTGTCATTACTGGTGTTTTCTTTTCCTTTTTGTTTTTTGGGTTTTTATTATCATTAT
TATCTGACATCTTTTTCACAGTAGGTTGATTTTCCACCGAAGGCAGTCCTTAAAGGTTTTCTTCAGAGCCTGGATAGAT
GGCTTCGCAGTTAAGAACACAGACCCACATGTAGCTCACAGCCTTCTGCAACTTCAGTGCAGGTAGTCTCCCTCCTTTT
TGAATCGAAGTGTGGGCCAGCAGTCGAGAAGGATTTATGTTTCTTCTTCTGTGCTCTTTGGAGAGGCACCGAATCTAGA
AGGCAGAGTGGTTTCAGGTGGTCCTGGCACGGCTGTCTCAGAGGGGGAGCTAGGTTTTGAGTGCCAGTTTCTCTGTGGG
CTGTGGAAGCTGTAGCATCCTGCTTCACAAGGCAGCTCACATGCCACACAGGCCGCAGCTTCACGGCACCGCACACTTG
TGCGTCAGTGCATGCTCTCTGCCCTCTGCAAGAATCTGTGACACGGAAGACATTTGGATGGCTAACTACGTTTCCTTCT
TTCCCCCCTAGGTCTTGATGCAGAGCTTTACTATGTGAGAAATGACCTCATCAGTCACTACGCTCTGTCCTTTAACCTG
CTAGTGCCCAGTGAGACAAACTTCCTGCACTTCACTTGGCATGCAAAGTCCAAGGTAAGCGCTGCCGGCAGACCACGTG
ACAGGTCACCCAGTCTGCAGAGGCGAGGGCTGTCCCTGAGGATGGGCCTTCCTCTCCACAGAAGGGTCAGTGGTGGTAT
AGAGCACTGGGTCTTTTCAGGTGTATAGCTGACAGATACAGTCTTGAGCCTGCTCCTCTGAAAGTTTAGATTTAGGTGG
CTGCCAGGTGGGTGACCAGCGCATCACAGGGCTGGGTCCTTTGAGGCTTTTGCAGGGAGATCTGTACCCAGGAAAAAAG
AGAGTTGGATCTGATGGTGACCCTGCTGTTACAGAGAAGCCATATGTGGCTGGAGGATGGGTGAGTGGCTGGGGAGGAG
CATGGCTGAGCTGGCTGGCTCTTCCACACACACTTGGTGACTTGAACGTAAGTTTCCCATTTACTGTTCTGAGTCCCCC
TGGTAACCATAGATACTGAAGCGGGGAGGGGGGTGCTTATCAACTTCTGCCATTTGTGTATTGGTGAATTCTTCACCTC
TGACTGCCAAGGAACCTTACATGCTTGTCTCCCAGATTTGCCAGATGATACTGTTTGTACCCTTTACTCCCCTCCCTGC
CTGCCTATGAATCCTTGAGCTGTTTCAAAGTGCATTGAACGCATCATGTCCTCTCTCTCCTAAATCAAAACCAGGAAGT
TTGGTGTTCTAACTCACAGCACTTGGGTAACTGAGTTCTAACTCACTCATCAGGGACCCTCAGCATCTTTATCACTAAG
GTTTCACTAGAGACTGATGTTGTTTCAGTTTGTGTCAATCTGAAGTTTTGATGCCCTGCCCATGTGCTTCAGCCTCAAG
TTGTTGGGAGTGGAGAACCTGTCATGGTGGAAGCTAGCTTGGAAGGGTCGTCTATGAATCTATATGCTTGCAGGGGGAA
CACTGAAGGGGACTGGTATAACTCTAATTATCTGGTAGATTCCTCTCCTCTCCTCCCCCCTCTCCCCTCCTCCCCCTCC
CCTTCTTCCCTTCCTCTTCCCCCCTCACCTCCCCACTTCCCCCCTCTCCTCCCTCCCCTCCTCCTCCTCTCCTCCCTTT
CCTCCCCCCCCCCCCCAATCCTTCTCTCTCTTTCTCTTCTTGAGGCAGGGTTTTACTCTATAGCTCTGGCTACATGTAG
ATTCAGCTGGCCTTGGACTCAGATATCTGCCTCCCAAGTTCTGGGATTAAAGGTGTGCACCACTACACCTGGCTAGCAG
CATGTATTTAACTATACATTTGGGCTTCTTCTTGATGATATTACAGTCCCCAACTCTAGTTCTTAAGTCCTAGTATCTG
GTGATTTTTGGCACAGCTGATTAATTGCAGTTTGTTTATTTAAAAAGTTAATCGTACGAGTAGGTAGTACATGGATATG
GCCTGTAAGGGTATACAGTGAAAAGGAAGGTTTTCCGTTGTCATGGTAGCATGCTGAGCAAGCCCATCTAGATCACTGT
CTTCATCAGGCATTGTTGCCTGAGTCTTGTCTCCTAAAGAAAGGTAGTGTATATGTGTGCAGTGTGCACACGTACACCT
AAGTGCTTCCTGCTTTCGTGTAGTGTGGAGTTGTGAGCAATGTGTCCTGGTAAAGGGGCACTGTTCCTTGTTCCTTGGT
ATTGCTTTGTGTGTTCCCCCCTTAAATAACTGTATCATTTTCTCACATAGTTGTGCTCTGGTCATTTCCTACAAGAGGA
AACCGAGACCCAGGTTTGGGAAGGACTTGCTTAAATGAGTCTGGAGGCTGAAGAAACAGGTGCTAGGGGTGAGCTCACA
TGGCAGTCACCCGAGAAGGCAGGAGTAGTAAGGTGCGGTCTCTTATCACTTGATTCTGAGATACAGTCAGACTTCAGAA
GGAAGGCTGTCTCTCTCAGTTTCTAACACTCTGTCAGAATCTCTGTTTGGGCTGTTTGAGCTCTTGCCAGAAGATGAAC
TGTTGGCTTCCGAGATCTCCAGACTCGCCAGTGTATCACTTGAACACTGCAGGCATTTTAACATCAGTGTTCCCACATC
AGAGCTTTTGCTGGTAGAGTTCTCCAGTGTTTACGTGACTGGTCTTGCCAAAGATTTCAGTTCCCTCAGCGGCTGGAGC
TGTGTGTGTCTTTTCTTAACATCTATGTCACGCGCGCCTCGCTGAGTGGTTGAGAATGTGTTAATTGTATTTTAAGATG
GGAGGAGAACTCTGGTATTGATTCTCTCACAAGAGTAGACGACTAGGCGGTCTTAACAGATAATTAGTTTCTTGTATGT
TCCAGAACATTCCCCAGTGTTCAAGAAGAAACTTGCAACTGTGTCTGCTAAGTGTCAGTAGATATGCTAGACAGCCAAA
AGGCAGAGGCTCAGTAGGATGTGGCAGAGGACTCCACCACCCACTCCCTTGGTCTCCGTCCAGGAGATTCTGCGGGGAG
CTTGTCAGAGCTCCAGCAGCTGCCTCTGGGGGCCAGCCTGCTGTGTCCCAGTGGAGTGAAGTAGCTGTGAGAAAGGCAG
TTCTGGCAATTTCAGAGAAATTAAAAGGCCGTTGACTGTAGGCGAGCACATTCACTTTAGATGTGTTCCAGAAGGGGAC
TGCTTTATTCGTCGTCTGCCTATTCACACAGCCATGCGTTCCAAGTAAGGAAAAATACCAAACATGTTGATTTTTTAGC
TTATTCAAATATTTATATTTTCTCTAGTTACTTTTCTGAAGTATTTTGGCTCATTTATTTTCTGATAAACAGCTGCTGC
ACAGATTTGCTTTGGGGTGGGGGGCAGGGTAAGGCTGGGGTGCACTTTCTAAATAGATCTCTTTCTTCCAAGGTGATTG
ACAAACGGCATTCCTTACAGATCAGTACACACCAAAGTAGAGACAATTGTATTCCAGTGATTGGCACTTTCTTCTCTGA
CTCCAAAAACTACATTAAAAAAAAAAACCAAAAAACAAAAAAACCCAGCTGTCCTCAGTACACCAGTGTGAGGGGGGAA
CTGCCTTCTGATGCTTCCGTGTTCATTCATCCGTCCAAAGGAAGTCTTAGGCAAAGGGCAGGCTTCCATGCTTCTTGTT
GTGGGTTCCTCTGGGCCACTGCTGATTCTCTCAGTCCTTCCCTCAGCCACCTCTGCCTGTGCTCTGCTCCTTGTTTTGC
CGCCACTTGCAACATCATTTCCTCCCTTTCTCCCTCTCCCGTGCTTAGACTGAGATGGCCCTGCCACTCCTCTTGCCGC
TCTCCCTCAGCTCTGCCGTCAGAGCCCTCTCTTTGCTTTCCTGGCTTTGGTCCCCGGACAGCTATTCCTCTGCTTATAG
CCAGAACTTTGTAAAGGAGACCGAAAGGTTAGAGCAGATGTCCAAGAGGGGCACTGAGGGGTAAGGAGCTTCTGGAAGA
GCATTGATGCCATGGACAGACAGAGTCAGCAAGATGACGTGGGCCTCAAGCACAGCTCAGGCCCTGGCACTAGAAACGG
ACGGCCTGGCAGGGTGTCTGGGGAGAGGCATCGAGCCCATGTGATAGAAGTGGGGTCTTTGATTCCCTGGCAGGAGGCA
GAGACAGGAGGGGGAGCTGGATCAAGTAACTCACTTCCCAGGTGAGAAATCCTGGCTCCAGGATGTCTGTTAGCTAGGA
ACACACACTGTCCCAGTGGGGTGCCTGCCTACCTATGAGAGGAGGCTCCCCCATGTATGTAGGAACCCACAGACAGTAT
CCCAGTGAGGCGCCTGTGTTGATCTGTGAGAGGAGCTTGGCAATCAGGTAAGCTCAACTCTGTCTGGTAACCCTACAGC
TTCAGCCTTTGAGGTTACCAGATGGGTATGCCTAGTAGGTAGTTGGAGGAAAAGGAGAGGGAGAGACAAGTTGAAATGT
TTATCCTTGGGACTGGAGTGCAGAGGGTTAATGTTCTGATAGGTCAGCCTGATGCAGAAAGGAATAAGAGTTCTTCCTG
GAAGTGAAGAAGCCGAGCAGGTAGCGTGTGGTGCACCAGCGAGGAGCACGGGTGAGGTTGAAGAGGAGCAGCCATTGAG
CACAGGTCTGGGGGGTCATCATGTGTATCATCTCCTCAGCAAGCTGAGCCAAGGGCCATGGGGTTGCAGGCACTGCCAT
CCAAGTGTGAAGAAGAAGGAGTGAACCAGCTGAAGGTGTCAGAATGACTGTGTCAGTGATGTCCCTGCTGGGGCACAGG
CTGGGGCACAGGCGGTGGGAACATGAAACCCGGCATCCTGGCTGTGCTGGGAAGGAAGGAAGTAGTTTAATTAACCACT
GCTTTCCCCCCTCTACTAGCGCAGCCCTCGGCTCATAAACATGCCTCATAAATGTTGTCCGACTTAGAGTAGTGAGCAC
AAAGAACTGCTTTTACAGGGACCAGAGGTGGGTGGTCAGAGGCAGTGCTGGCATCTTGGATGGAATGGACGGTATTTTT
GTATCTGTTTTGGACATAGAGAAGTTGAGTGCTATGTCAGAACGAACACTGAATTTCAGAGTTAACAGAAAGATGGGAG
GAGACGGAATGAGCCAGCCTTCGAAACCTGTGTAGAGTCTTGTGTGTGACATGTGAGTGGCCCGAGACATGCACTGAGT
AGAAGAGAGCTTCAGAGTAGAGGGGCTGATTTCACGCAGGACGTCTAGAGAGGGCCTTAGAGCCTGACAGGGATACTGA
CCACATGTGCAGACCCAGTGCTCAGGTCTAGAAAGTAGACATGAGCAAGAAGAGGAGGAGCCTGAGGGGAAAGCCACAC
TGAGGGAGATCATGAGGGAGATGAGAGTATGAGAGGCTGTGCAGCCAGTGGGGTTCCTCACATCAGGAACAAAACCGAT
CCCACCAGTGCTGACAGCTCAGGAGGGTTTGCTGCAAACCCCTGGGGTTGGCAGGAGGCTGATGACCACAAAGGAGTGC
AGCTTGACAGTGGGAGAGGCTGCTGAGGCCCCGGGGTCCCGAGCCAGTGTCACGGGCCTGTTGTGTCATCAGCTTGGAA
AAGTTGCTCATTTGTGCAGGTCACGTGGCCTCCTGGTTCTAGATCCCATGCTGATCTGTCTCAGGCACCACCCCCCCCC
CCCCCCCAAGCTATGGCCTCGTGCAGAGATAACAGCCATACCTTACAGTCAGTGCATCCTCTTGAGGTTTTAGTGCTGG
GAAGCCCAGGGTACCTTAGTGATGGCTCTCTAGCGCCCCTTGGTGGCAGCCATGGCGATGTGATGAGCTGAGAGATTGA
ATGCTTCCTTCCAGACTGTTGATTTTAAGGGAAAGGGTGGTTCAGTTCCAGGTACCAAATGGACAGTAGCCTTCAGGTT
GAATTTCTGTCTGGCTGAAAAGTCCTAGTATGATTTCAGTTTTATTATTTTTATTTATTTATTTTTGAGACGGGGTCTC
ACTTTGTAGCCCAGGTTGATCCTGAACTCACTATATAAACCACACTGGCCTCAAACTCACAGAGATCCACCTACTGCTC
ACGGCTGACTGCTAGAATTAAAGGACTGCTCTTGGAATCTTTTTGGGAGTGGATTTTTATTCCACCCATGAATAGCTCT
CATACTGCTTAGTTAATGAACTTACTAATGAAGTCTTTTTCTGTTGTCAGGGGTATAATGTTTTATCTCTCTTCATTTT
GCAGGTTGAATATAAGCTGGGATTCCAAGTAGACAACTTTGTGGCTATGGGCATGCCCCAGGTCAATATTTCTGCTCAA
GGGGAGGTCCCACGCACTTTATCAGGTGAGCCAGGAAAGACGGACTTAGCATACTGCTTTCAGCTGCCTCCATCCTATC
CCTACAAGGATAGTTCACTGAGTACCGGGCCCTCCCACATCAATCATTAATCAAGAGAGTGCCCCACAGACAAACTCAA
AGGTCCCAGGGGGTTGGGAGGCAGGGGTTGGGGAAGTAGACGTGTAATTATTATTGTCATCTTTATTGTCTATTTATTT
TTAAAGAATGTTGTAGGGCTTTTCCTGGCAGTTTTATTCACAATTATTATTGGCACAAGTTCAGCTAGGCTTTAAGCAG
AGCTGGGAGGCAAGTTAATTTTTGGGGGTCAGCAAGAGTTTCAGAAATATCAAAGTCTCTGATCCACTACTCATAGATG
GTGAAATATCTTTGAAATTTGTAAGAAAGGAATCATAAATTAAAAAGGCAAAATGTTCTAAAATCTACGACTTTGTCAT
TAACATTCCTAGATATACCTTTAGAAGCTAATCCTATTTAGAAGGGCAGTGTTGTTTTTAATTCATAGCCAAGAACATG
TTAGCTTTGTTTATAGGTTTATTATAATTTCCCCCAGTAACAATGTCTTTATTTCCTAGAGGTACTGCTTTTGAATGGT
ACAATTTTTATTTTATTTTATTTTTTAAAGATTTATTTATTTACTTATTGATTCATTTATTTTATGTATATGAGTACAC
TGTTACTATCTTCAGCCACACCAGAAGAGGGCATCGGATCCCATTACAGATGGTTGTGAGCCACCATGTGGTTGTTGGG
AATTGAACTCAGGACCTCCGGAAGGACAGTCAGTGCTCTTAACCACTGAGCCATCTCTCCAGCCCTGATACGGTTTTTA
AATGTGGTTACTAAATACACATATGACATAAACATTCTCCACAGGGAGTACTTCTAAACATGTCATGCACCTGTCTGCT
CATACTATGGATATAGTTGTTATTGGTGGTGTGCTGACGTGTATTTTTGGCCATCTGTTATGGTGTGGCTTCAAGCCCA
ACAGGAACTCTCTGGCACCCAGTTCAGTGCAGCTTTGGTTTCCCTACATTTTCTTAGCCAGACAGCAGGAAAGGCTTTC
CCTCCTTGGGATTCGATGATGGCATGCTTTATTCCGGGTAGCTCATGTCTGCCCTGCAGGCCGCAGTCCACACGGTGTG
GTCAGCACTGCCTTCTTGACATTCATACCCTTCACGTTGCTAGGAGCGATACACAGGTGCATTTGGTGCTGCAGAGTCT
TTCCTTCCGTGGTGTAGTTTTGTTACCGTTGCTGGGGAGACTGGTAAGCTCAGCAGTTTGCCTCGTGATTCCTGAGATT
CAGTGCATTGGAAAGCAGAACATAGACAACAAACTGATATTAAAAACAAAGTCACAAGGAAGGCTCGGAAGTAACTATC
CCTTCATCTGTGTCCTGTGCTCTGCTTCAGTCCTCTGGATCTGCAGTGAAGGAAATGCAGGTGTCCAGAGCGGTGGTGA
CAGGGTCTCCGGAAGCTCCTGTCTGATCCGCTTATTTAGATGCGCTTCTTTATTTTATTTGAGCAGAAATAGATGAAAA
TGTTGTATAGCATTGATCCTAATCTTTAGGACACTAAAGTGTTTTCTCTTGTTTCTTTTAACAGTGTTTCGGGTCGAGC
TTTCTTGTACCGGCAAAGTCGACTCTGAAGTCATGATTCTAATGCAGCTCAATCTGACAGTGAATTCCTCAAAAAATTT
TACAGTTTTAAATTTTAAACGAAGGAAAATGTGCTACAAAAGTAAGGAATTTATTTAACAGAATGTGTTTCATGTAGAT
AAGAGGTCAATAGAAGACTGCATTTTCTGTATAGAGATTTAAGCATGACCAAGTCTTGATTATAGTTATGAGAAAAGCG
CATTAGTGGATGAAACTCTGGGTCAGCCTCTGGCTCTCATTCCATTGGAGTTCTGTGGGTGTTTCCAGAGCGAGCGACA
GAGCTGAGCATTCGCTCGGTGGGCTTGGACTCTGCAGGACCTTTGTGTTGGGGGTCACATGGGCCTGGGTTGAACCATA
TCTCTGTGTTTTCAGGACAGGGGAGATATTGCACCTCTGCAGGGCTGAGATTCTTGTCTGTGAGATGTGCATGTTGCTG
TTTCAGAGAGGTTTTAAAGGGTAACGAAACCTAGTCCAGAATTAGAAAGTCACATGTGGGCACTTAATAATTAGGGTTT
GAATTATATCATCCATTACCCAGGAAATCACCATAGATAGTAGTGAGTTCTAGAACACCAAGGCTGAGTTTTGAAATGT
TAAGATTTTGTTTTAAGAGTTAAATATTTTATGTATATGAGTACACTGTAGCTGTCCTCAGACACACCAGAAGAAGGCA
TCAGATCCCATTACAGATGGTTGTGAACCACCATATGCTTGCTAGGAACTGAACTCAGGACCTCTGGAAGAGCAGTCAG
TGCTCTTAACCCCTGAGCCATTTCTCTAGCTCCAATTTTAAGATTCTTAAAGGTTATCTAATCTCCATGTAGCCTACAG
ATAAAATTTCCAGAATTTACAAATAGGTAGAATTGTTTACTGTTTAAGGTAGAAACAAAACAAACTGTAAACTCAGTGG
ATGTTTCCCGTGATCTGTACACTTTACACGCGCTAAACCCAGCTTTAGGTGTTATGTTTCAAAAAGTCTGAGTGGCCTG
ACCAAGGTCAGACAAAGTGCTGCAGCCTGTGCTCTCCACTGCTTTGTGAAACTATTTACAGTTTCCCAGTTGAAACTAG
CAAGTGAGTTACAGTGGCAGATGCCAAGATACAGGCCAGAGATACAGGCCAATAAAGAAGCTATATTGCATTGAAAAGA
GACATTTTTAAAATTAAAAAAAAATTAACACCCTTAGCTTTAGTACTGTGAATTATATACCTACTTCAATGTAAGTTTA
AAGCCTGTAGGGACTGTAGAGGCTTTACTCCAACCTAATCTTTTTGTGATTGGCATGAGGACACTGGTCCTCGAGAGTG
GGAGGCCTCCTAGGTTTGTATAACTACTCAGTGGGGATGGATGGAGGATGGATGGATGGATATAGAACAGGTGGATGGA
TAGGTGGATGGATAGATAGATGGAAGGATAGAGAATGGATGGAGAGTGGATAGATGCATAGAAGGATGTATGTATAGAT
GGATGGATAGAGAATGGATGGATGGATGGATAGAAGATGGATGAATGGATAGAAGGAAGGATAGAAGATTATGTATATA
TGAATGGAAGGAAGAAAGGATAGAGAATGGATGGATGGATGGATGGATGGATGGATAGAAGATAGAAGATAGATGGATG
GAGGATTAATACTAGGGTTGGACCTAAGGTGTGCTAGGAGAATAGAATGCTTTACCATTGTGCTATATCCAAAACTCAT
ATTTTAGAATTTTTATTTTGAGACAGGGTTTTGCTAACTTATCCAAGCTGGTTTTGAACTCACTATGTAACCTAGTCAA
GCCTTGAACTTGTGTTTCTCCTGCCTCAGCCTCTGGGTAGCTGAGACTAAGGTGTGCACCACAATGCCTGACTTGTGAA
AACTGTCCCATTTTAGATTTAATGCATATCACCATCTTAATTTGGTGGTCCTATTTTGTCCACTTGGATTGTGTAGTTT
TTGTTTCTGACTCTTACTTTTAACATGTGATTTTGTTGTATTTATCTGGTTTCCTAGCCTATGGCACTTAACTGAACTA
GTAAGCATCCCCGAGCTGTGTGGAATAGCTGTTACTTCTGGGAGGGGGTTCTGTATCTTATGGGTGAGAATTACAGGTC
AGGAAGTTTCCTAGAACTCCAGAGCCTTTCCCCTCAGCTGGTGGCGCGGTTTCCATGATGACAGTATAAGTTTGTGGGT
TTCCCTACACTCTTTACCACAGAAGCCCATCTCATTGGGAATCTGTAAGTGACTGGCAGAGACTGTCCTTTGACTAGTG
ATTGCACTGATGGCAGGACAGCCGTTCTCATGATCTTACTGCAGACAGCTGTGACTTAGCTCACGCAGTGTTCAGGAGG
TATGTTAGTATGTAAGGATTGTTACAGAAAAAAACTTCTAAAGTACTTTTTATTTCTCTTTTAATCCAGAACTTGAAGA
AGTAAAAACTTCAGCCTTGGACAAAAACACTAGCAGAACTATTTGTAAGTGTCACTGCGTTCGCGTTCTCCACCCTGCG
AGTGTGAGCATGACTGCGTGAGGCCTGAAGCCGGGTGCCAGCACCAGTGTGCTGTCTGGTTTGCAGAGTGATATGTAGA
CCAAATGAAGCTGTTTGAGAATTCAAACTTAAACATACTTTATAGCACGCTAACAAATACCTTTTAGAAGCAAGAATTT
ATATTGTGGTTTTTCTCAGGCAGGACCAATAAAGGAAAGGATATTAAAAAGTTATGTGTGAGACCTCCATTAGCAGAGT
GTTTAAGAATCTGACTTGTAGCCTGGGCCCAGCTCTTAACTAATTTCATCTAGGTGAAAATGAAAATTTATGTCATTTA
TTCTATATTTTATTTTGATATAGCGAGATGACTTAGTTGGTAAAGTGCTTGCTATGTAAGCATGAAGACTTGTTCCGAT
TCCTAGCACCCGTGTAAAAGGCAGGTGCTGCGTATACTCCTGTAATGTTGCACTGTGGGGGATGGGAAGGTGGGAGAAG
GGCAGAGGCAGGCAGATCTCCAAAGCCTCCTGACCTGTCAGTCTAGTTAGCTAATGCATTCCAGGTTCAGTGAGAGATT
CTGACTCAACAAGCAAGGTAGGAAGTGATAGAGGAAGATACAGAATGTCAGCCCCTGGTCTGTGCATACACATGTACAC
ACATATGCACACACATAAACATCTGCGCACACACACACACACACACACACACACACACACACAGGGGGGGGGGGAAGAC
AGACAGACAGACAGACAGACAGATGAACACTCTGGTTCAGGCTGGTCTGGAGATCTGTATGTTGTTGAGGGTAACCTTG
AGTTTCTGATTCCTCTTTCTCTACCTCAGGAGTGGTGGGAATTGAACTCAGGGCCTCACACATGCTAGGCAAACACCTA
CTAACTGAGCCACACTCCCAGTCCAGGCATAGATTATTTAAATACATGGAGAAAAAAATTTTGTTAGGTCAGTTAGATT
TATTTATTTGTTTACTCTTGGTATTGATTATATTATACACCATTAACAAGCTTCTTTTTTCTCTTATGAACACATTATG
GTAGCATTAGCACCAAATGATCTTTATAGAAATAGATAATATTTTCAGGAGAAAGTTGCAAGTATACTGAAGTTCAGAG
TATGCGCTGGTGGACATTTATGTCCTCAGTAACTATTGCATGTGTTCTTCTGTGCATTGCTAACTCCAAATTTGATGGA
TAGGGGATTAGTGTCTTAAAGCACAGTCTAAACATCACATCATTTGACCCCTAAGTATGTCACTGTAGGTCACCTAAAA
AGCCCCTAGTTACCCAACACACCCACATGTTGATTTATCACATATATCATCTCTTCTATCTAATCTGTGTTCAAATTTA
CATAATTGTGCCAAACCATCTTTCAATAGTTTCCTTCTTCAAAATCAGGATCTAAATGGAGCCAATACATAGTGTGTTT
TGTCCATGACACACACACACACCACCACCACCATCTCTTCCTCCTCTTCTTCCTCCTCTTCCTCTTCTTAATTTTTTTT
TATTTTTTATTTTATTTATTTTATTTATGTGAGTACATTGTAGCTGTCTTCAGACACACCAGAAGAGGGCATCAGATCT
TATTACAGATGGTTGTGAGCCATCATGTGGTTGCTGGGAATTGAACTCAGGACATCTGGAAGATCAGTCAGTGCTTTAA
CTGTTGAGCCATCTCTCCAGCCCCACACCACTTCCTTCCTTCCTTCCTTCCTTCCTTCCTTCCTTCCTTCCTTCCTTCT
TTCTTTGTTTCTTTGTTTCTTTCTTTCTTTCTTGCATTTTTTTAATTTATTTTTTTTAATTAGGTATTTTCTTCATTTA
CATTTCAAATGCTATCCCAAAAGTCCCCCAGACCCCTCCCCCCATTCCCCTACCCACCCATTTCCACTTCTTGGCCCTG
GTGTTCCCCTGTACTGAGGCATATAAAGTTTGCAAGACCAAGGGGCCTCTCTTTCCACTGATGGCTGATTAGGCTATAC
ACCACTTCTTTATTAATTGATCATATTGTCTTTTTTTCCCATAGAATTTGCCAAAGTAAACATTCTTCTTGTATTATTT
AATTGCCTTTAACTCTTTCTTATAACTGGTAGTTAAATTTTATACTTGATTAGACTTAGGACCACTTCTTTTGGTAAGA
GCAATTCACGGGTGTCCTGTGTGCTTCTTATTTATTGTTTTAGCACCTCAGAGCACCTCAGAGCACCGAGCATCTGTCC
TAATGTAGGCTTTTGTTTGTTTGTTTGTTTGATTGTTTCTGTTTTTGTTTTTCTCTGTGTAGCCCTGGATGTCCTGGAA
CTCACTTTGTAGACCAGGCTGACCTTAAACTTTTTCTCCCCTCCCCTCCCCTCCCCTCCCCTCCCCTCCCCTCCCCTCC
CCTCCCCTCCCCTCCCCTCCCCTCCCCTGTTCTCTTCTCTTCTCTTCTCTCTCTCTCTCTCTCTCTCTCTCTCTCTCTC
TCTCTCTCTCTCTAATTTAAATGCTCACAAGGTATTTCTCCCTAAGAACAACAGTAGGATGCTCTTGGCCTCTGTCTCT
CTCTTCCTTGCTCTTTCTCTTCCAACTTTTATCACTCCTATCCAGCATTTACTGAGTCTGCAGTATACTAGGTGGTGTG
ACTGAGGCAGAGGATTGAACTGAGAAACAGCCCCAGCCTTTACGGACTTAAGAGTGTTCCGGGGGAGATAGGACAGCAT
GGTAGGCACCCCGAGGAGGGACTTGTCACTGCCTCTGAAGGAGAGCGGCAGCAGCTAAAGGAAGGGCAGCTGTGTTCTG
ACAGGGGAGTGTCAGAGAGGAGTAGGAGCAGAGCTACACCCGCTGGAAGCTGAGGGGCTGGGCAGAGTGCGGGCGGACG
GCCTGCCCAGCGGACCTGTCTGCTCCGGCCACTGCTTGGGCTGCAGTGTCCATCCACGCAGGCTTGGGGCTTTTCTACC
CCTCCTGAGACGTTTCAGCAGATACTCCTGGGAGTCTTGTTTTCACTTGACCCAAATCTAGCTGTCCCACTTGCATTAA
CCTTTTAGATTTTTATTCACTTTTTGTTCTTTTACTCCACGTTTAATTGTTTGGTTGTTGGCAGCTTAGGATCCGTCTG
CTCAGCCTTCTTCCCAGTGTTAGGCTCCCGAGCTTGTGCCGCTGTATCCAGCCTGAGAGAAGCCTGGGTTCTCCTTTAC
TGTTAAAAAACAAATCACATATGTATACAATGTATTTTCATCTTATCCACTCCCCCTATAGTTCCTACTGGGTTTCCCA
TACACACACACAATCCCTTCCCGCCTCTGTCTGTGTGTGTGTGTGTGTGTGTGTGTGTATGTGTGTGTGTGTAGTATGC
TCACTTAAGCATTCAGTTAATTTTTCTATTCCTGTGGTGTTTCTTAAAATAATGTAGCTGGGTATAATCATATTTAATC
AATAGAACCCTTTTAAAAAATCAAAGAAGCAAAATTATATTGATCAAGAGAACTGTGCAGTCTGCCCAGCAGGGTGACT
ATTAGCTGTGTGTAGCTTTGAGGCTATAAATATATTAACACAATATAAAGTAGAATACAGTGAAGTTATTCTGGTTGTT
TTTCATACCACATAGTTAAGGTAGCAAGTGGCTACTGTGCAGGGCAGTGCATTCCATCTGTTTCTGCAACTATGCTATA
GAACATTTACTGAAGGACATGCGTGTCTTGTATACTGGGGTGTTTTGTATCTACTGTTTGTGAGGGTCTGACTGTGGTT
CATATTAACCAGAAATTTTAAAGTCTTAAAACTGTAACAAAACCACCAAAATTGGTGTGAGTACCGCACTGCGGGTATC
TGGTTTTATCAAAGGACGGGTGTGATGCAGTGCAGGCCTTCCCGTGAGGAGCAGGGCACTCCCCGTGTTAGGCCACAGA
GCACCCCATCAGCTCTGTAAAAACCTCACGTGCCTCTCATTTCTGTTTCAGATGACCCTGTCCATGCAGCGCCAACGAC
TTCCACGCGTGTGTTTTACATCAGTGTAGGGGTTTGCTGTGCAGTGATATTTCTTGTAGCAATAATATTAGCCGTTTTG
CACCTTCATAGCATGAAAAGGATTGAACTGGATGACAGGTACCGTTCATGTTTTGGGGAGCATGTTACTTGCATGTGCA
TGAGTGCCCCTCCCCCAAGCCCCCCCCCCTCCATCCATGCACATTGTGCAGGGGAGGAGGGAGAGTGATGAAGCTCAAG
CCAAAGAGGAACTCAGATGTCTCGGTTATTACTGCCTTTATGTTTTTATCTTCATCTTTTAAAGTTTGTGGAGTTATAA
CCTGGGGGAGAAATAAAGGTCAGAAATGTTTTTCATGTTTGTTATCCTGATTCGCCTCCAGGTCCGACTGCCTTTTTTA
AACTACTTCCTTCTACGTCCTCCAGCCCCTCCCCGTCCCTCCTTCCATCCCTCCCTTCCTCCCTCCCTCCTCCCTCCCT
CCTGCCTTCCCACTTGAGTATCTGCTGTACTTTAAAGTTTCTTGGATGATCCTAATGTGTAGCAGAGTAGAGACACCGG
GTAGATAATTACACTAAGTGTTGGGACTATAAAGATTAATGATACTCTTCATGGTATCAAGGTTCTTTGATGTTAACCC
ATAATTATAGTATAGAATTGATGATTGTGGTATGTATAATGTAGTGTGGGAATGCAGAAGGTCCTGGCTTAACCTGGGT
GAGTCACACACATTATAGCCAGACTATAGTGAAGGGTAAAGATGGAATGGGAGATACGTGCTCATAACCCACAATCCTT
AGCGACAAAAGGCATTCTCAGCATTGTTTTTGACTTTGAACTTTGAACCCTTTCTACCTGTCTTGATCCCTAGTTGCAA
CTTGTCAGGTGATTCCATATCCCTGATGCTGCCCTTTGAGAGCTTTTGTTCATACTGGGGCCATGGGACTTCTTGCTAA
TCCAGACCCATTATTTGTACTCCAGATCCATCACCGTCTTTCCCACAACTAAAATAGAAGCTTTTTCCAGGCAGGGAGG
AGAGACCCTTCTCTCCCCAAACTCTCCTAACACTGTACAGTAGAGCCATGATATCAGCATTCCCTTTGTAAAAGATCTG
GGGGCTGGTTGGATCCAGCACTCTTTAAGTACTTTGGATCCAATACTCATGACTTAGTCTCCACTGTGCTCCTTCCCTT
CCTTGAGTCAAAAGAAGAGAGGAGTGAACATGGCGGAGTGCATACAGGCACTCTAACCTGCTTTCTGTTGCCGTGATAA
AACACTGGCCAAAACCATTGTGGAGGAGGGATGGGTTCATCTCAGTTCACACTGTCAGGTCATAGTCTAGGGGATGTCA
AGGTAGGAACTGGAAGGCTGAACCATAGGGCATGGCTGCCACTGCCTTGCTTCCTTAGGCACTGAAGGCCCACCTGCCT
AGGGGTGAAGCTCACGGTCGGCTGGACCCTCTCACATCTGTCATTAACCAAAACATCTCGCTTACATAGTCACGGGCCC
ATCTGATCTGGGCAGTTCCTCAACTGAGACTCCTTCATGAGACTCTAGGCTGTGTCAGGTTGGCAGCCAGCTCACTAGG
GTAGTGGATGCTATGTTTCAGAGTGTTTGGTGTACACCGGGATCATGGAGTTCTTTGATTTTACAGAGGAAGAACTGGC
ATTTGGAAGCCGTTGAGGGGAGAAAAGACTAGACATCTTGTAAACAATTTCGACAGATGATATAAATGATGAGAGTTAC
TGAAGAGTAGAATAGTCAGAAGTCATACTTTGTGCTTTTTGTTTTTGGAGGCAGGGTCTCATGTAGCCCAGGCTGGTCT
TGAACTTTTGATTCTCCTGCTCCACCCCCTAACAGCACATACTGTTATACTCAGCTCCACAGGTCTGATACTCATCGAG
TTGAGTTTTCCTTGACTGCTGGTGTGTGTGTTCAGGCCATGGGTTCACACAGGCCGACATTGTGCTGTAACTCATAGAT
TCCATGAAGCATACGTGTCTTCCTGCTAAGTATTCAGCACTCACAAGCAAACTCTCTAGGTTTCTAGATACTGTCTGAA
TAAAAGCAATTATGTGATTGCTTGTTTTCAGGGCACAGCCTAGTAGTTAGGAGCTAGCCTCTGGGGCCAGGCCATCTGT
ATCTGATATCCAGCTGTTCTATTTACCAGCCTCTCAGCCTTCTCACGTGTAAAGCAGAGATTGTGGGCTGTGAAGAAAG
GACGCTAGAGGGGTGGGTAGCAGATCTCAGCCGTAGCTACATGCAGTAAGTGCCGCCATGCCGACGTCATCCCCAGAAC
GATACAGTGGATAATCTGGCGTTCCCATTATTCCTGGCTTGCTTACAACACACCAGGCAAGCTAATGGTGGTGGCCAGC
ACTGGAGACACCTGCTGCTCCTCTCTACACTACATGGTTGTGTAATTAAGACTCAGAGACAGACGTAAAGACAGACTTG
TGGGATTGTGGCAATACACCAGTACAAAGCAGGAGGGCTCCATTCTGCCATTTCTGTATTGTAGTTACATCAGATGCTT
GCTGCCTGACAAGCTCTGTTCCTCGGGAGGCTTAGTGGTGCTGTGAACTCTTGGGAAGAGCCAGTGTTCTGTTACATAT
GAGGTTTCTCTGGAGGCAGAGGGTGTAGCTGTCTGAGACGTGCTCTCAGCAGGGACACATGGAGTTCGCGCGCGAGGCT
TTCCCTCTCTCCCAATGCAGCACATTGATCTGTAGTCCATTACAGCAGAGTCTGCCCTGAAGGGGCTGCGTGGTCTCCT
GCAGAGCTCGGTCTGTAGCTGCTCTCTCTGTGGTCTCCCCCTCAGGGAATGGAAAATCCATTCCTCGAGTATGGGGTGC
TTGCATGTGTTATTTGGTAGTATTTTCTTTTTCTCTTCTACTTACTGCTTCCTAGCACCTCACTACCCTCAGCATTAGG
GTGGGAGTGGGGGCTTTTTGTTTTTTCTTTTCTATATTTGTTTTTACCCTTGCTGTCGTAACAAACTGGCATACATAGT
AATATACAGAGTCTTAAAGTTTCCTCTTCCTTCTCCTCCTCCTTCTCCTCCTCCTCCTCCTCTTCCTTCTCCTCCTCCT
CTTCCTCCTCCTCCTCATCTTCTATCTTCTTCTTCTGACAGGGTTTCTCTGTATCTGTAATTTGCTCTGTAGACCAGGC
TAGCCTTGAACACAGAGATCCTCCTGCCTCTGGCTTCTGAGTGATGGGATTGAAGGCGTGCATGAAGAATGAAAAAGTC
GGTTACTGCGCTCAACCGTTCCCTCCGCATCACGTATCAGCAGCTGGAGTCGGCAGCAAGCATTCTGCCTGGCCCCCGT
CTTCCCAGTCCATGGGCGTGGCTGCTTTCACTGAGTAGCTAACCCACTGTGCTTTGGGCTCTCACAGGGGTCCCAGTTT
GCTTCCTGTGCTGGGCACCGGTGTAAGTGTTGGGGAGGTGGAGTGAGCAGGCACTTAGCTGTCCCCATGGAGCTTGTGG
TCTGCCCAGATGAGACGTTCAGCAAGAAGTCCCAACTGGAAAGAGGGTGGGAAGGTCTGTGGTGTTCAGTGCTGCCTCC
TCAGCAGCAGAGCACCTCCTGTCACACACAGACACTTGATAAATATTTATTGAATAAATATATAAATAGATGACTCACT
GAAAATAGACAGTAAGATTTATTATTTTGAAGATCTTTGTTTAAATTCTTAGCTTATCCTAAATCTAGGTGCTTTCTTG
CTTATGAAAAATCAAATTTATTTAGACTAGTCATATGCTACTAAATGTTAGTAAATATACATTTTAATCTGTTTCCTAC
CTCCTGTGATGGTGTCGGGCTGTCCTTGCTTCTCTTATAATCGTACGTTTGGCTCCAAAGAAAGTTTCTTGCTCCACTG
TTGCTTCGCTCTTCTTTCACTTGTTCATTTCTGTTAAGGAAATGAAACTCTGACATAACTAGCCATCAAGCTTGACTGC
TCTCAGTCATTTTTAGCTGACGCAAGAGCACCACCTACTGTTCAGTTGTCAAAGTTTACTCACCCGGACACATTGTTGC
TCGCAGGGAATATCTGAGTTTTCACAGATATCATACATTTGAGCCTCACCTCCTGTGAGGCAGGTCTCCTCAGTCCTCT
TCTGCGGAGGAGCTCTTATCTGTCCCCTTCTGTGATGCAGCCCTTCCCTCCTCTCTGCTGAAGACTACACTTCTAACAA
ATAGGACTTTTAGTCACATCTTTGCCAAGTACTCTGCAAAAACATGTACCTGTAGGTCACCGTTCTAAGGTTGGAGACA
TGATGCGCAGTATTCCTCATGATAAAAGAGTGGTCGTTTATAAATGCATGCCTGAGGGCCAAGCAGTGACCTCAGTGAT
GTACTTAGACATCTTCCGTGTAAGACCCTTTTAGCCGTCTTGTTGGGAGTAGCTTTATGCTTCCTTCTATCCCCACAGA
GTTCGAGGCTCCAGAAGGATTGAATACTTTGCCTAAGCCACACAGCTGGTGATGTGGGCAATTGTGATTTACAGGTTTA
TGCTCAGCATTTGCTTATGACCAACTACATATGTAAACTCACATAAATCACCATTTAGTAACTTCCAGAACTCCAACCA
CACAGTAGCCTGACAAATCTTTGTTACATTCTATGAATTGAAAGTCCCCTCAGCAAAGTTGACTGTAAGTTCCCAGGAA
GAAATAACAATCTGAAATGTGAGCGTCTCCAGGGAAAGAAGGCATGTTGAGGGCACATTAAATGAACAGGCATTTCACG
TGGAGATTGTACTGTGCAGAGTGGCTCATGTGCACACAGGCTCTCTGAATGTTTATTGGAGGACACCTCTATCTCAGTA
CTTCAGCCATGACAGTAGGTCATTCTAAAGTGTGTTAGCGATAAAAGGCTACATAGCCTCCAGAACTCACTACTTAGAT
GCCTTATGTCTGTACGTTTTTCACATGTGTGTTTGGGCTCTTGAAATTGGCAAAATACAAATCCATGTGTAAGTATCCA
GATTATATAGCAGGTGGTCACCAGTTCCATTGTAATCACTGGGGTTTGAAACTACGATGATAAGGTACAAATATCAGTC
AGTCACAAGGTTCTTCATATGTAATAGTGGGCTCCACCACCTCTGTGGTGACAGGAGTTCTGTCTGGCTCTCTGGTTTT
ACTCTCTGTGCTGAATCTCTGCTTTAACATCTCCTCGGCTACAAAATTAAAAGGAATAGGCCAGTGAGATTGAAATGTT
CCCCAGAGTGACCTGGGACAAGGTTTCTACCCATTGAGAAGATGGAAGAATATGCTTGGGAAGTGTTGGTTTATAGGAA
TTACAACACGTGAGTGACAAGATTTGCGCCCACCACCTTCAGGGGCTCCTCTTACTTGGAAAGTATGGGGTGATGTGAT
GTTTCAAGTTCTAGAGATTTCTGAGAACCATGAATTCTAAAAGTGGCATGGGAGGGGTGAAGAGGGACAACATATATGA
TTAAACAGTGAGACTGTGAGGACACCCCTGCCCCCGTTGAGCATGGCACTCTTGATTTAATCTGTGGCTGCTTCAACTC
AAGACCATTTGATTTGGGCCATTGCTGAAATGAACTTGTTTTTCTCCTTTTCTGAAAGCCAGTTCAACTGAACAGCGTG
GGGTTTTTTCTAGAAAGCCGAGACATAGTTCCTGACACACCCTGTGTGAGGTTCAGAATTGCTCTCTGAGCACGTCTAC
CCGCACTCCTGAATAATTTTGCTCTGATGGCATGCCCTCCGTCGTCACGTCCCTCAGGAATCCAGTTTGGAACGCTGAT
AGAGATGCGTGCCTTTATTGCATTGTTATCTCGACCAACTTATGTACCTTATATGTATGGGAAAGACATCTCTCACTGA
GGCTCACTTATCAAGTATTTCTGTCTTCAAATTCTTTGCAGCATCAGCGCCAGCAGTAGTTCCCAGGGGCTGTCTCAGC
CGTCTACCCAGACGACCCAGTATCTGAGAGCTGACACACCCAACAATGCAACGCCTATCACCAGTAAGAGTCAATTGAA
TCCTAAAGCTGTATGTGCTCTGTCTGTGGTTAGAGTATTTGTGGGATTAATGTACCAGTGTGTAACTTGTGTAAAAGTT
GGTTTGATCTTCGATTTTGTTTTTTTAGAGCACTGAGTTAAAAAAAAGTTGGGGTATTTAAAGTAAAAATAGTTGCTAG
AGCTAGTTTTGCTTTGGGGAGATTTCCATAGTTAATTTTATTAAAAGAGCTAATAAATCAATTTTATACAAATCCAGAC
TGAAGGTTTTCAGGTTACTTATATGGAATAAACTAATACTCTCATGGGGTACATAGAGACTTGGCATTGGTTTTACTCA
TCTGATGTTGGAGATAGAAGCCAGGGTCTCCAGCATGCCCGCTGTACTGTGTAAGGAGCTGTCCACCAGTCCATGCTGC
TGTCTTCTAATCCTAATTCAAATCTCATTACCATTGATATGGTCTGCCTGTCTGTCTATGATTTTTCAAAACAGGGGTT
CTCTGTGTAGCCCTGGCTGTCCTAGAACTTGCTTTGTAGACTAGGCTGGCCTCAAACTCAGAGATCCTTCTGCCTCTGC
CTCCTGAGTACTAGAATTAGAAGTGTGTCACACCACAGCCCAACTTTTTTGTGGGGGTGTTTGGAATTTCCTTTCCTTT
CCTTTCCTTTCCTTTCCTTTCCTTTCCTTTCCTTTCCTTTCCTTTCCTTTCCTTTCCTTCTCCCTCCCTCCCTCCCTCC
CTCCCTCCCTCCCTTCCTTCCTTCCTTCCTTCCTTCCTTCCTTCCTTCGTGTGCCATGCTGGAATCAAACCAAAGGGCC
CTCACACATGTTACAAGTACTTTACCACTGAGTGACATCCCTAGCTTTTAATTTCTCTCTTTAAAAAGAAAATCAACCT
GTGTGTCTGTGTGTCTTTATATACATGAGTTCTAGTGTCCACAGATGCCAGAGGTGTGGGGTCCCCCTTGAGCTCGAGT
TCTAGTCAGTTGGAATTGAACTTGGGTCATTTGGAGGAGCATTGAGTGCTCTTAGCCACAGAGCCATGTCTTTAGCCCT
AATTTTCTTCTTATTAGCAGAATTTTGTTTTTTAGCTTAGGCTGGCTTGGAACTCTAGCATGTAGATTAGGTTGGCCTT
ATATGGGCCACCATTACCAGCCCATTTTTATTTTTATACACTTCAGGGTTAGGAATTTTGGGGAAAGTTTTTTTCTTTT
TTCTTTTTTTTTTTTTCATTCTTTCTTTTGGTTTTCTTGAGATAAGGTTTCTCTGTGTAGTCCTGGCTGTCCTGGAACT
CGCTCTGTAGACCAGGCTAGCCTCAAACTCAGAGATCTGTCTTCCTCTACCTTCTAAATGCCAGGATTAAAGTTGTGCA
TTATTACTGCCCAACTAAAATAAATCTTTTAAAAATATTTAACTGACAAGTTATATACAGTGTGATATAAAATATGCAT
GTAATAATGTAGTGTTATTGGACTATACTTTAGATATAGTGGGGAATCAGTTATGAGATGATGATGAGAATAGACTAAC
CTTCTGAAACTGTAAGCAAGACCCCAATTAAATGTTTTCTTTTATAACAGTTGCCTTGGTGATGGTGCCTCTTTGTAGC
AATAAATGACCAACACGGGGAGGAAGATAGCCTTGATCTAATGTTATTACCAAGAGAATGTCAGTGTAAGGAAGACTAT
TGATGTCCTATAATTAGTAGCCTTTCCAAGGCAAGACCTGAGCCAGAGAGAGAGAGAGAGAGAGAGAGAGGGAGAGGGA
GAGGGAGAGGGAGAGGGAGAGAGAGAGAGAGAGAGAGAGAGAGAGAGAGAGAGAGAATGCCTAGACCTGGCATGGGCTT
TTGAAGCCTTAAAGCCAACCCCATACCTCCCAGTGACACACTCCTCTAACAAGGCCATACCTCCTAATCCTTCTAATTT
TTACAAACAGTGCCTGGAGACTGTTTAATTATGAGCCTATGGAGGCCGTTCATACCCAAACCACCACACTCTCAGTGCC
CTGCGGTGGTTTTTCTAAACTGTTTCCAACAAAAAGAGTCAAACGTGTTAGGAGAAAAAGCAGATTCCAGGTCTGGGTC
AGGAAATGAACAAGGCAAATCTAAGACATATGATTGAGCAACATATCCCACTTACCAAGGATGGAGCCACTGAGTATAC
AAGAGAATAATGACTGCAGTGATTTGGAATAAAATATGTGTAATAATCCATAAATGTATAATGCTTATAATTGCTCAAC
ATAATTATTAACTGGGGCTCCATCAATACCTTAGTATCTGACAGCTGGCTGAATGAATTATATTGCAGGGTAATGAGGT
AGCACCAGTTGCTTAGAACAAGCATTTCCTTACAGAAGGCTCCCAGCTAATGACTATGAAAGGAATGGTACAATTAGAC
AAGGACATTTAATAAAATACTTAGTCTAGGAAAGACCATCATTGATGAAAGCCCTAAGCACAAAGGAAATGGAGGCTTC
ACCATTGGCCAGTCAAATCCTTCCCTGATCCACTGATAGATAAACTTAGCACTACAAAAAGTGCAACACCCAGGTATTT
TATACCTGAGGATATGAAGTTCCAACCCCAGATAATTAGCCTGGATTTAATCAAGGCTAGAGCTTGGAGCTGTCCTGTA
ACACAGAGACAGAGGAAGAAGGTGAAGGACATGCCAGGAGCAAGGAGGGCAGTTCTGCATCATTGATTGTGGCAGGAAA
CAAGGGCCAACAAAAAACCTCAGAGAAATCAGAGCAGCACCTTTGGTCACTCTCAAGAAAGAAAGATGACAAACCCTTC
GTGAAAATGCCGTGGTAGGTAGGAAGGTCATTTCTGCTCAGAAAATGCAAACCTTTAATGTCGCTTTTAATGCTGACAT
TAGGGCTGTCCTTACTGATGGATATGACCTTATGAAAGAGACTATATACCAATGAAAATAATTTACATATTAAAAATTT
GTTCTTAAGGTGAAATTTACCTATTTTTTGTTTTTCACTGTGGACATTATCACAGAGAAACACGTAGTTCATATATCAA
TAGACGCTAAAAACACTGGAAACCTTTAACTTTTACATGTATGAGTGCGTTGCCTGCGTGTGCATGTGACCATGTGCAT
GAAGTCTCTGCAGAGGTCAGAAGAGGGCGTTGGGTCCCCTGGGACCAGCCTTTACAGCAGTTGTAAGCTGTCGTGGGTA
CGGGGAATCCCACCTGGGTCTCTCTAAGAATATCAAGTGCTCTTAAATACTGAGCTTTCTCTCCGCCCCACCCCCAACA
AATTGTCCTAAAAACTCAATATAAATACAGTTGTTTTATGACTAAAAAAGGAAGGCCAGGCATGGTGGTACACACTTTT
AATCCTAGCGCTCAGGAAACAGAGGCAAATAGCTCTCTGTGAATCTGAGGCCAGCCAGCTCTACATAGTGAGTTCCAGG
CCAACCAGGGATACATAGTGAGACCCTACCTCAAACAAAACAAAATAGGAATAGCAACAAAAGGGATGGGGTGTAGTGG
ATCATACCTATAACCCCAGTATTTGGGAAACTGAGGTAGATCATGGGTTCTGAGCCAGCATGGGCTGCCTAGTGAGACT
TGTCTCAAAAAGAAAAAAAGGGGTGGGGGCAGAAAACAAACTTAAATACTGGGCTTCGGGAGTGCTAACGAGAGTGGTC
AAAGTGAAGATGTAGTTTATCAGAAGTGATGCTTTTCAAACTTCTCCGTATTTGAAAAGATCGTTTGTCACGTGATTTA
TTGAAAAATCATTTGGGAGGGAAAACTAGAGAGGGATTAGAGTCTGTAATTCAAACCTTGTGTCATTTGTCCCAAAGGC
ATTCCAAATGACAATAATATTTTTAGTGCTTCTTGTTTTAACATTTGGAAGTAGTGGTGTCATAGATGGGACTGTTTTT
CCTTAATTTTTTAAAAAAAATTATTTTTATTTTATTCATACTGATGTTTTGCCTGACTATATGTCTGTTGTGAGGGTGT
CAGGTCCCTGGATCTGGAGTTAGAGACAGCTGTAAGCTGCCATGTGGGATTTGAAGAGTAGACAGTGCTCTTAACTGCC
GAGCCACATCTCCAACCCCTTCCCTTAACATCTTATGTGCCCTTCAGCAGCAGCTTCTCTGATCTGTGTGTATAGTTGC
TTCATTATTCATTTGCTTCATTATTCTTTGCTATGACTTAAGTCCACAGGCTGGAGTGCCTAGGTGAGCCCAGTGCTGC
AGTAGGGTATTTATATTGCTTGGTCTGGCTTTGTTGGCTCCTCAGGTTATCCTACCTTGCGGATAGAGAAGAACGACTT
GCGAAGTGTCACTCTTCTGGAAGCCAAAGCCAAGGTGAAGGATATCGCAATATCCAGAGAAAGGATCACACTGAAAGAT
GTCCTCCAAGAAGGTACTCGTGCTCGCTACAGTTTCAGTTCTGTTAAAAAATTGTCTTCATTTTCTATTTCTTTAGACT
TTATACAGTGTCGTAGCAGCTTTGATGAACATCTTGATTTTGAAGTGTGATAAAACAGACAAGTATTTGTGACCTATAA
ATGTATTGTATCCTTATTTCAAGTGATCAGTACCCAGCACCTGTATTTACAATACTTAAATTAGTTTATTCTTTCATGG
CTTGTGGATGATCAGGTGTGTGGATGGGCATTGGGGTTAGGCTCAGCGTTTGTAGCCTGAGTGACCTTAGAGAAACCAC
ACAGTATCTCAGAATTTCTGTAGAGTTCACACCATGCTAGTCTTCATCACTCTGAGACTAGCTGAGAGGCGACATGAAA
TGGTGCATTCGGAAGACTTCTCAGAGTTTGTATTTGACATGTCCAGGGCTCTTTTCACCCCGTCTTGCAGGCCCTTGAT
CTCGGCTTTGAGTTTTAGGTCTCCATTAAGGAGATGGTGAGATCGGAGGACAGAAATTAACAGTGAAATACTGCATCAC
ACGCGGTTAGATGGCAGATGCTTGTTTCCTCGCTGCAATTTGTTCTGGAATGTGTTTGGTCCGCACAACTGTTCTGTTT
GCTGGGCCTCCTGGAAGCTAGTCTCAGCTAAGTGAGGACCTAAGATGCCTTCTGAGAGAGTGGCGCTCAGTTGACGCAG
ACTCTTATTTCTGTCTGTTCTTCAGAGTAGGAACACTGCTCCTACGGTTGTCTGCTTCCTAACTCAAATAACTAGATAG
ATCATCTGCAGTCTGAGGAGAGAAACCAGGCCAGACAGCATTTCCTACAGAGTTAGTGGTTATGCTTCCAATTTCCTGT
GTTTCCAAACAGGATGCTCATACTTTGAGGAAATTTGGAGCCATGTTTTCCTTGATACTTTTTGGAGCCATAATGGTTT
TTTGTTTGTTTGCTTGTTTTTTGTTGTTATTGTTTTGTTTTTTAAGAACTTTAGCTTTCAGAAAAGAAAACATGACTGG
CTAATATTTTGAAAAGTGGTGAGGTTCACTGGAGATCTGACTAAGTACAAATCAAACAGTGGTTACACGCCACTTTTCA
TCTGTTAGATTGGATGAAATTAAAAAGAGATAGCTAGAGATCCAGATGGGGTTACAAGGTGATAAACTGTTATGCACCC
TGACCGGTGGCAAGGTAATCTGTGTACACATGTGCTGTAGTGTGGGGTTTGCCCAGTTACTTTTGCATTTAAAGTTTGT
GTTTGGAATATCTAAAGGGGACTAGGTTTAGGGAAAAGGAGACACAGCAGGGTGGACGGAAGGACCAGGTCTGTCCTGC
TGGGCAGGGATAGAAGGAAGGACCAGGTCTGTCCTGCTGGGCAGGGGTGGAAGGAAGGACCAGGTCTGTCCTGCTGGGC
AGGGGTGGAAAGAAGGACCAGGTCTGTCCTGCTGGGCAGGGGTGGAAGGGAGGCACTCCTTTGACAGTACAGAGACTCG
GAGGTGTTTGGACAGCTCCAGGCAAAGATTCTCCAAACCCTTCTGCCTGCAGCACCGATGTCACTCAGACTCTGTGACT
CAGTGGGACCTTCCACATGAGCACTGGTACCTTTAGCACACACAGTGAGATATTAAAGCCAAGGTGTGTGTAGTGTCCA
CAGTAAAGCTAAACTCTTTCAGGCATTTCTTTTAAATTATGAAAAAAAAAATGGTCTGTGCGAAAGAATAGCATGTCAG
CAATCTTTTTTTTTGAGGATCATCTCATTTAAAAAGTATTTTTAGCAATCAGGAAATTAATAATAAATCTGTAAGGATT
TTTGCATTTCTGTTTTACACTGCTGTGCCCCTCGGGAATGTCATAATTTCTTTGCTGTGAGAGCTCCTTAGGAGCACAC
TTGGCCCTCAGTGACCTGAGAGTTGGGTCACCTCACCTGAGACTGAGGGGTAGAGGCGCCGAGGCCTCTCTGAGCCAGG
GTTAGCGTTCCTCACTCTTTCATCATTGAAGTCTGCATCGCAGAAAGAGCGGCCGTTCTCTCCCTCCGTCGTTGCTTTG
AGGCAACTACCAGAGACTCAGATGCTCTGGCTGGCACCTGTAACTGCGACAGCACGCCTGACCGACTGTGTTTATTTAG
AGAAATTAAGGAAAATAGTCTCAGATATTTAGTCTAGCTATAGGATTTCTCAAGCAACCTGCTTTGTGGGCTTGAATTG
CTCTTAACAGCTTTAGTCCGGTTTATTCTGGAGGGAGAATGAACAGCAGCCACACCTGTCAGCTTAGTCGCGGTTACTA
GAAAGCTTTGTGATACTGTGAAGATTATTGTATGGCCTCATTAACTGTTATTTTTCCTTTTAGGTACTTTTGGGCGTAT
TTTCCATGGGATTTTAGTAGATGAAAAAGATCCAAATAAAGAGAAGCAAACATTTGTAAAAACAGTTAAAGGTAGGGAT
TTTTACATGCTGTTTTCATAAGAGTTTTTTGTTTGTGTTTTTTTGTCAATCACTCAAACTAGCAAAACTCTGTCCCCTG
TCTCAGTGTGTGCCAGACCTTTGGAATGACTCTGTACTAGTGTGACTCACAGGCTAATTTTTGCAAATTAGGTTTTTCT
CACCAGGTATAGAAATTAGTTTTCTAACTTACTAAACATTTATTAGAAATGAATCCAACAGTAGTTAAATTGCATAATT
GCATGCGCTTAACCTATCTGTGTCTAACTTTGAGGTTTTTTTTCCTGTTGAAACTCCCTTATACCATCCTTAATCATAT
GTAAGAAAACATTGTCATTAGTTTTCAACATATTTATAAAGAAGATAATGAGCTGTGCTCAGATCCACAGTGCCTCATA
TAGATATTTTTTTACATTGTTTACCTTTTGTTCTCTCCCCCACTTGATCTCTTACACGCTGTGTAGTGCCCTAAGACAT
GCCTGTTTCTTCAACCCTTACCCTGACTGCCACCCTTTACCCTTACCCGTGTGTTAGGTAAGCAGGTGTTCATGTACAT
GTGTGCATACATGCAGAGATCCCAGGAGTCAGCCTTAGGATTCTCTAATCAAGTGTTATCCGCCTTTTTGCTTTCTTTT
TAAATTATCTTTATTTTATTTTAGTTTAGTTTATGTATATGAATGTTTTGCCTGCATGTGTGTATTTGTACCACCTGGG
TGCTCTGGGCCCCTGGGAGGGTGTTAGATCCCCTGGAACAGACAGTTGTCAGACACCAATGGGTAGGTGCTGGGATTTG
AACCCAAGTTCTCTGGAAAAGCAGCCAGTACTTCTAACCACTGAGCCATCTCTCCCCAAATCCTCCTTGATCTTTGAGA
CATCTCTCTCTCTTTCTCTCTGGGACCTAGGGCTTTCCAGTTAGGCTAGGCAGACTGGTCAGCGAGCTCCAGAGATCCA
CCTGGATCTGCCTCCACAGCTCGGGCTGTAAGCACGTGCCAGCGTGCCCCGCTCTTTTTCATGACCATCAGATTTGCTA
GGAAGGCAAGCGCCTTACAGACTAGTCCTTCTTTCCGATCTAAGGAGTGCTCCTTTCTGAGAAAGAAACTAAGCACTTG
GCAGAGATTGAGAGGAAATTATTTTTGAGTAGAAGATGTAAAGTTTTTTTGAGATTTACAGTAACTGTTTCTAAATGTA
CGTAAGGGAGTAGGTGGGAAGGACGGAGGGCTTATGTATATTATTTCTAAAACTTCAGGTTTTACTAGTCATAACTAAA
ACATCTAGAATTTACTGCCTGTAGTTTAACAAAGTTGCCCGTTTAGAACTTCTACAAATCCTTAAACCCCTCCGCATTC
AAACGGTTCAACTCGCTGCTTTGTTTTGTGTTTGTTTCCTCCCCTTAGCTCCCAAAACATTTAATATTTTGTTAGACGA
TAGTGGGCTTTCTGACTAATTTTGAAGGATAATTTACATGACTCTTTTTACATTTTACTCTTTATAATTATAAGGTTTG
AGCCACAAGTATTGTAAAGGAACTGCTTTTTGTTTTCTTATTTTTCTTAAGGAAATACTTTATTTTAATGGCAGTACTT
TTGGATTTATAAATACTCCATGCATCATAGTGAGTCCTGAATTCAATCCTGAGTTAGTGTGGAAAGGAAATAAGAGTGA
TAAGAAAGCGCAGCACAGATCCTGCAGCTCTGTGATAAGCCTTCGCTCTGTGCTGGAGCTGTGGCCACCGCGCCCCCCC
CCTCCGCTCCCCAACCCCAGTAACTACTGTGTTCTCCCTGACGGGAGGGAAACAGGAACAAACACAGTGAGTCCTTAGC
ACTCAGCCATGGCTTATGTGGGCTGGGGACATGCAGTGGTGCTCGTTTGCATAGGCCACATGGCTGAGGTGCTCCACGT
TTATCTTTTAGAGTATTGATTCTCTGTTGATGCTGTGTCCTGCTGACAAACATCCCTCACAGGCTGGTGAGCTCTATTT
CATCTTAATGCGTGCTGCACAGATAGTAGCTCAACAAAGAATAGGGTTTGGGGGTTTGGGGTTTTGTTTTAGAGAATGC
AAATTTTTATGGGGAAAATTGAAGCCTTTTTTTTTTTAAGTTTAATAATTTTGTTCAGAGATCTAATAGATATCCAGTG
TCAGTTTCCAAAGACTGAAGTCAGAAATGTCACCTGGATCAAAACCCATGAATTTAGATACTAGAAATAAAATGGAGCC
AGGCACAATACACCGTGCTTAGGAGGCGGAGGCAGGAGGATCGGAAGTTGGAGGCCACCTTAGGCTACATACTAAGACT
TTGTAACAGTGCAGCAAAGTGTGAAATCTAAGTTCTCTCATCCCCGGAGCCCACAGAAAGGGTGTGACATGGTTCTTCT
CACCGTCAGGGGACAACATGGTCCCCAGGTAGTTTTGATTTTTCAAGTAACTTTGTTTTTAAACCGTAATGTGCCTTAT
GGAACCTTACCAGATTCTGTCATCTGCTCCGGAGTTTCTGTGAAAGCTGCCGTCTCCAGCCAGGGTCTGTCGGCTGCCC
TGGGCTGGAGGCTGCTTGTAAGTTACAATGAAAATTGACCCTAGGTAACGAAATGTTAGTGTAACACGTACAGTATTAA
TCACGGCAGTGGTTTTTGTTTTTGTCTTTGTTTTTAATACATAGACCAAGCATCTGAAGTTCAGGTGACGATGATGCTC
ACCGAGAGCTGCAAGCTTCGAGGTCTGCACCACAGGTGAGAGCGAGGCTGCGCTTCAGCACCGAGCACAGTGCGACTCC
GCACACTTACTCACTTTTTAAGAATGTTATGTCTTGTTCAGAACTGAATTCTTTTGGAAATTCTTTATTTGTTTTCCTT
CTGAGTATAAAGGTAAATGCGTGCTCATTGAAAGCAATTTGTAAACAGCAAAGGACGAAGATGGTAGAGAACAGGATGT
AAGCTATGTAAATAATAAATGTGCACAACTAAGCTTTCTAAAAGAGTCTACGTATCATGTACCTGTCTCCCTTTAACAA
ATATGACAGGGTGATGCATACATTAAAGAAAATTGCTACAAAAATTTATATCTATGGAACACCAAGGCCAAAAATAATT
AACCATCCATCCTTAGATACAATGAGGTTAATGGTCTGCCATGTACTGTTGAGTGTGTATGCATGCCAGTGTGTGTGTG
TGTGTATGTGTGTATGTGTATGCATGCCCGTGTGTGTGTGTGTGTGTGTGTGTGTGTGTGTGTGTAACTACCTGAGATC
TTGTATCTAGAAATAGCCATCTATGTATTTGTCTGTAGAGATATTTTATAATAAATTTAGTGTGTTTCCAGTGTACTTT
TAAAAAGGACAATGTTTTTTACCTTTAATAAAATTAATTTCTTTATGTAACAATAAAAAAAAAACCTGTTTCTAGGTTT
TCATGTTGGTAACTGTAAATCCCTGTCCCCATTTTAATAGGCCACTTAGTGGTTTACTGAATGACTGTACCACGTTTTA
TTTAATTTATCTCTTATGTATGCTCTTAAAATCATAGTATGATTTCACAAAATGTACCTATTGGAAGAACTTTTAACAA
CTTCTTCTAGACCAGGAATGATTGGGGACAGCAGCATCAGTATCAAGGCAAAGTTTTTGCATGTTTGTTGACATAGTTT
CTATATAGCCCGGGCTCTTCTGGAACTCACTATTTAGACTCCACTGTCTCTGCCACCCGCGTGCTGGTATTAAAGGCAC
TTTCCACCAAGCTGAGCTGGCAGTCGGGGTATGTTTGTTGGTTTGTTTGAAGCATCATATAGAGAGGTCTAGGAGGTGG
TCATGTCTCCGACCCAGAGTTGGAGGGGTACAGACAAGTAAATACCTGGAGCTCATTGGGCAGCCTAGCTGAACCATTG
TGTTCCAGGCCAGACAGAGACCAAAGTGGATAACAGTTAAGGAAGGCACATGATGTTGACCTGTGACTTCTATGCACAT
GCTTAGACTGTACATACTTATATATTCCTACATGTATATACACCACACACAAGTATTGTGTAGACTCTGCTTAGTTTGT
ATGACTACTGAGAGTTTCACATATCTGAAAAGTGAACCCTGACTCTGTGTGTTAGAAGGCTCTGTGGGCTTCTGGGAAG
GCCTTGTGATGTGTGCAGAGGGCACCATTGGCTTCCATTTGGACTTGGGCTGTTACTGAGCTCTGATAAAAGTATGAAT
ACACAGAAATTCCTTCTTTGCATTTTGTTTTTACTTTGGGTTTTGATGGCCTAAAAAGATGACAGCCACATGAAAATGA
GTTCAGTGGTAATGAAAACGAGACAGATATTTGGCATTGTGGTGGTGTAAAGAAAATAAGCCAAGAGGAGAAGACAACT
GTTTGGACTGAGATTAAACACAGCATAATTCTTGGCTTCTGATCAGCTGAGATGAAGGTGACATTAAAGTCTCTAAGTC
TCAGAAGCAGGGGGAAGAGAAAGCGCGTTGTTAATAAGCTCTTTTAGTCTCAGATCCCAAGACGGAGGTTTGCATTAGG
TTCATTATTTAATGTAGATTCTGATAATATAAAAGTAAATATTCATTTTCTAAAACTCCATTGAAAAACGAGGCTTTTG
AAGAAAAAGTTGAACGCAATATTGAGAAACAGTGAGGGTATTCCTTGGAAGAGTTGAGTAATATAACTCAAGAATGTTG
CCTTCAATTGGCCAGATTCAGCCCCAAGAGTAGAAGTCCTCACTTGTTGGGAAATAGACAGCACCCCCCCCCCCCGGCT
ATATTTGCTTTAAGAAAGGAAGGAAGGAAAGGAAGAAAGAAAAAGAGAGGGAGGGAAGAAAGAAACTAAGTTATATTTG
CTAAAAGCCAGAAACACGTCTGGAGCTTTTAGACCCTTTTTAACAGTGTTGTGAGTGAGTGCTTGATGCTGACGCAGAC
TCTAGAGGAAGCACCTTCTGGCTGATCACTTATGCTCCCGGCCAGTTCCCTGACCCTGTGAAGCTGTAACCGGCAGGAC
AGAAGCACGTGGCAGGAGTTAATGCAGACAAAGCCACAGACTTTTCCCAAGTAGGAGCTTTAGATCTACCCTAGCCCTG
CGCATCCCTTGGCTGAGCAACCTTCCAGGCAGGCACTTACGTTCTCTCATGCAGCTTTAATCTGCACTGCTTCACAGCA
TCCCTCCATGATGGGCTAGCAGTCTCAGACACCCCACAGAAGGAGAGGCGAAGCAAGTTGGTCAGAGTTCATGTCATCT
CGGCTGTGAAGTGACGTTAAGATCCCTAGTTGTGGCAGTAAAGCTGCTGGTATGCTCTATTCCCTGTAGTCCTCCATCT
GACCACACGCAGTTAATGGAATACTCAAGGCTGTGCGATTGCTTGCTTCAATTTCATTCACTAACTGCCCGTTTGTACT
TAGGATTCAGGTTGGTGTAGAATAATAATAATAATAAAAAAGGTCATCTGGTTTTTTGGTTTTTTTAAAAAAGATTTAT
TTATTTATTTCATGTGTATGAGTACACTGTAGGTGTCTTCAGACACTCCAGAAGAGGGCATCAGATCTCATTACAGATG
GTTGTGAGCCACCATGTGGTTGCTGGGACTTGAACTCAGGACCTCTGGAAGAGCAGTCAGTGCTCTTAACCGCTGAGCC
TTCTCTCTAGCTCAAGAGTAATCTTAAGATCAGTACCATAGAGTTTATTGTGCAGGCTCAGTGCACTCAGCAAGAAATG
TCACCTGTTTTCAGGTGTCACCTTTTCATTGGTTGTAGTCTGTGAGTCAGAAGGTTTGCTTTGTGTTTTTACATTCTGT
GATACAGGTTAAGAATCCCTGACACGTTTGAAAGCATCTTGACATTCTAATTTTTTTGTTAGCTTTGGTTTTTCTACTG
GAACTTGTAAGTTTTCTCATATTGAAATGCTTGCATAAATTAAAAATAAACTGCTTTCATTACTTCATGTCTGAAAACC
CCTAAGCCGTTTTTTGTTTTTTTGTGTTTTTTTGTGGTGTTTTGGTTTTTTTGTTTTGTTTTGTTTTGTTTTTGTGGTT
TGAAGTCCCCATTGGCCCAGAATGCCCAGTGATGGCTGGCCCTTGTTAATGCATAATGACAGGTAGCTTTAGGGTTGTA
AGCATGCTCAGCATGGGAGGCAGTTAGGCCCAGCCCCGAAGCCCTGCAGCCCCACCTCGCCTCCGCCTTCTCACCTCTC
CTTTACCTCCATCTGTCCATTCTTCGATATCTCAGACCTTTCTGTTTGTCCTCCTTTCCTTTGTAAAATGGGCAGTGAA
GAACAGTCATTTATTCTTGGAAACATTTTCATAAAGCTTTTTACTATTTCCAGAGTTTTTCCAGGTTTGGTTTTTGTGT
ATATGGGTGTTTTGCATGTATGTGTGTATGTCTGTGCACCACATTATGCGGGGCCTGAGGGGGCAGACGAGGCATTGGG
TCCCCTGAGACTGTACTTAGAGTCAATTGTGAGGCACCATATGAGTTCTGGGAATCAAACCTGGTCCCTCTGCAAGAGC
AGCCAGTGTTCTTAACTACTTAACCACAAGAGTAAACATTTTGGTTTTATTTATCTTATTTGTTGTGTCTGGACAAATG
CCATTAATTCTGCTAAGCAATTTCAGTCATGGGTGAAGGGACAGAGATAATCCTTTAGGGTTAAGGAAGTGGCCAGCTC
ACCTGCTTATCCTGAAGCAGGGTTAGCCCTGATTGATCCTCTAGGTGAAGCTCGAATTGGAGTGAGTGTGTGTGTCTGT
GTGTGTGTCTGTGTGTGTGTCTGTGTGTGTGCTTCTGGAAAGGAGGGGCTCTGCAGTCCTTCCTGCATTCTCCTTTATC
CTGGGAAGGAAATGTCCAGCAGCCCAGGGGAAAGCACAGGGGCGTAGAATCTGATGACACCTAAATTTCCTTCTCTTTA
TACTTGATCTTTGTCCATAGGTTCCTAACCACTCTTCAGCTGTAACGCTAAAGTCTTTGATGTGTCCGTCCAGTGCCTC
TTAGAGACTGATGCTTCTGGCAATGGGGACACTCTTAGGACACAAAGGCCAAGCAGAAACTTACTGTCATCTCACAAGT
CAGAGACAGCGCCTCTTGGTACATTTCTGCAGCTTCCCCTTTGTCTTTGTGGTTGTCCTGTGAATGTGTGGGTTGTGCC
TCAGTTTCTTTGAGTTCTTTTCATTTTTTTAAAGATATTTATTTATTATATGTAAGTACACTGTAGCTGTCTTCAGACA
CCCCAGAAGAGGGCGTCAGATCTCATTATGGATGGTTGTGAGCCACCATGTGGTTGCTGGGATTTGAACTCAGGACCTC
TGGAAGAACAGTCATTGCTCTTAACCGCTCAGCCATCTCTCCAGCCCGCTCTTTTCATTTTTTGATCGTTCAGTTGTAA
GGTGTTACTTTGTCATTCTTGGGCAACTTTTCACTGTTGATATCCTATTGACTTCTCTTGTTAATGCACCTTTTTATGT
TTCATATTAATCTTTTAAAAAACTTTTTGACATTGGTGTTCTGCCTCCAAGTGTGTCTGTGTGAGGGTGTCAGATTCCC
TAGAACTGGGGTCACAGACTGCCATGTGGGTGCTAGAACTTGAACTTGGGTCCTTTAGAAGAGCAGCCAGTGCTCTTAA
CCCCCAAGCCACCTCTCTAGCCCCCATGTACTCATTCTTTATGAAGCCATAAAGAACTAATAAATAGTGCTTTTAGGGT
ATTGTTATGTATGTGTTTCAAAAATGCATTAAATGTAACCACACAGGAGGTGGCACTGAGTATGTAAATTGAGTCTATG
TTATTTCTGGCTTAATAGAGAGGAGGTAATTATTTAAATACTTACAACCTTGGGGAAAGTGAGTTATTTTCATATATCA
GCTGGAGTGTCATTACACAGGAGGATGGGACGTGTGTGTGTGTGTGTGTGTGTGTGTGTGTGTATATGAGTGCCATGAG
AATTAAAGTATGCAAGATTGTTCAGTGCATTCTTATTTATTGTGTACATAAGAATTGTTTGAAATTCACATGTTACAAG
TTTGAAAACTAAGGCCAAGTGGGGGAGGGGAGCAGGGCCCCCATGCACTCCCATATCCACTCTCACAAGCATGCACAAA
CACACACACACACACACACACACACAATCACTCAGATAGTGAATCTTGTTTACATACACACACACACACACACACACAC
AGAGTGAATCTTGTTTTCAAAACTCAGGTTCCAAATTCTACTGCAAGCATTTGACTTTGTGACAGTTTCAACCTGTTGG
AGACTTTTGATGGTCCCGTTTTATTTGCAGCGGGGATGGGGCCTTTTTGATTTCCAGCGTGACAGTAAAGTCGGTGGGA
CAGCGTGATGATGAGCTCCGGGCCAAGCTGGCCAAGCAGCCATCAGCTGTCTGCGCCTGGCCTTTAGGGTGGGGATTAA
CATGGCCGTCAGCAAATCTAATACAAAGGAATTCATTCTGGGTAGTCATTTTTATTCTACAAAGAAAGCTCATACTTAT
GAGGACTTTTCACATCTGGTTTAAGAAGTAGGCCATTTGTAAATAAATATTTTAATGATAATCTTTTTAAGTGTGTTTG
GAATTTCATTTTGCTTTCGATTATCTTTATTACTTTTTATTTAAGATAAATCAGTAAGGTTTTAGATTTCACTTGCATT
TTTCCAAATCTCTTATTATTTTCTAGAAACCTCCTTCCTATTACTCATGTGTGCATAGAAGAAGGAGAAAAGCCCATGG
TGGTATTGCCATACATGAATTGGGGGAATCTTAAATTATTTCTTCGGCAGTGCAAATTAGTAGAAGCCAATAATCCACA
GGTGAGAAAATTTCCCTAAATATGAAAAATTCCTAATGGTTTTGCAAGTAGATATTAATAACCACACACATAGTGTCTA
AAGATTAAAAAAAAAAAAGACCTCAGAATGAACCAGGAGATATTTATAAGCTGTCGATTTAGAGGACCGTGAAGATGGC
CTGTGGCTTCCCAGTGGAGCACTCTTGGCCCATGCTGTTTCTCATTGTTCGCCACACCTTCATTCATAAATCACCTCTT
ATGTCCTATGACAGCATCTCAAAGTCTCTTCCGGAGTAACTTCTGAAGACGGCCTAGGATTTCCTTTCTTGGAATAGAA
ATCATAGCAGAGCGGTCTAACCATATGGTGCCAAGGGCACTGAAGGTTGGAGGTCAGGATCGTGGGGGATAATCAGGAA
TATCCTTAATGTGCCTGACTGTGAGACGCCACGGTTTAAGTTTCTCTCTTGTCACTACCGACGTGCTGTGGTAGTGATC
AGAGAGTCTGTTGGGTGCTTTGCTGTCTGCTATATTTTCTGTTCTTGTTAAATTGATTTTAAGACTTAAAATAGCATAC
TTCTTGTAGTTAATAAGAAGAACAAACTCAGATCTGCACTGAGTATTTGACTCTTAATGTATATGATATGTCGTTTAAG
CTAAAAGACTAAAGCTCTGGTTAGAACCATGTAGTTAGAGGTCGTGGTCTGTGGGTGTCACCAGGAGAGGAACTTCCCT
GCCCAGCCTGCAGTGCCGGCGGGCATCTCGAGGAGCGTGTCTGCAGAGAGACTGGTTTTTGGTAGAATGCTGCCCCCAC
CCCAGGGTGGTGGTGGTGGTGGTGGTGATAATCTGATAAGACCTACTACTACGAAAGAAAAGAGCCAGTCACTAGAGAG
ATACATGAAAATGATAAGTGTCATGGATAAAGTCTCATCGGTGGTAAATCCTGCTACCCAACAGGGGCTTTTGTAATGG
ATGAACAGTATTCCGTGCTGCCAGAGCACATCTTGGTTGAGAAATAGGTTTTCAAAATCTCAGTAAGTAGGTCCAAGGG
AGTGTTATGGGGTGGGACTGTAGACTCAGGTAATCAGATCCCAAACTAGTTCTGCAGTATCTACAGACTGTGTCATTTC
TTTTCCTCTGTTACCACAGGCAATTTCCCAGCAAGATCTGGTCCATATGGCTATTCAGATTGCCTGCGGGATGAGCTAC
CTGGCGAGGAGAGAAGTGATCCATAGAGACCTGGCTGCTAGGAACTGTGTGTAGGTGTCCGCCGCTCCCTGGGCTTGCC
CTCTGTCTGGGAGTCCCGGGGCACACAGACTGTTTGTGGTGATGCTGCATCTTAACACCCTGCTACCTTCTATGTTCAT
GTCTCTCTCTTCAGCATCGACGACACTCTTCAAGTCAAGATCACAGACAATGCCCTTTCCAGAGACTTGTTTCCTATGG
ACTACCACTGCCTAGGGGACAACGAGAACAGGCCAGTGAGATGGATGGCTCTGGAAAGTCTGGTTAATAATGAGTTCTC
TAGTGCTAGTGACGTGGTAAGTACTGGAGCCCAGGTCAACAGCATGTAGAGAAAAGCTTTCTGTTTCACTACAGTCAGT
GTTTTGTTCTTTTAAAAATCTTGGTGCTGTTCCTTGAAATTTTCATCCAGAGAGCCTGTGTACTTAGCTCCCTCTTGGC
CCACTTAAAGCAGAGCACCGGCCCAGATCCTGTGTTCTTGCTCACACCAGCTGAGGCAGCACCATTCCCACTGAGGGGC
GCTGAGGTTAGGGAGCGGGCGGCCACTATCATCTCCTTGCACAAGTCTCAGGATGCCTGAGTGGCTACTAAAGGCAGAA
TGGTTGCTTTCTGAGCATGCTCTCTTCTCAGTCTGCAGCCTCTCTCATCTGCAGTCCTGACACTGTTCCTGTTAGGTGT
CAGCGTAGTTCTTTCTCCTCTTCCAGAGCACAGAGTTGAGCCCTTCTGCTCAGTGATGTGCTTCCAGAAGCCGCATCTA
CTCGTCCGTTGTAAGGTTCTGTCCCGCTACTTTTTATTCTAGTCTAGATCATAATCAGCCCAGACTTGGAGGCGCGGGC
CTATCGTTCCAGACCCACCTAGGGTACATAGTGACTGTGTATCAAGAAAGAATGAAAAAGTAATATGATTTTTGTTTGT
TTTGTTTTTATCTCTTGACTGTTGCACACTGTAGCTGTGTATTTGGAAATTAGATTAAAAAGTTTGGCTATTAAGGTCC
CTAAGTCATCATTTGACAATACTACATTACTTGTAAAATAATTGGGATTTGATCTCATGTTTCAACATCTAAGACCTGG
GTTGATTGTGTTTGGGCGTCTGGCTTATTTTATTATTCAGAGCTGCCTTTCCATTTCGGCTTGTATGCTTCACACGTCT
CCCTTTAATAGTGGGCTACTGACACAGCTCCTACTCGTTACTTACATTGATTAGTGCACATGCTTTCCCCCCGTGTATT
GATTTTGCTGGCTCTATAACAAATTGCTCAGGGCTCAGATATAAATGGACTAGACATTATAAGTGTATATCATGCCTTA
TAGTTTTCATAATTGTTATAATTGTATTCAATTTATATCTGAGAGGAAAAAGAGCCTTTTGTTGGACTAAAAAGGGGAG
TATCGTTGAGGTTTGGCCTTTTGCTATGTACTGTGATGCTAAATATCAGCTCCCAAGGCCTGCTGGACCTCAAGGACTA
GAGAATCTACACAGAGAACCGACTGACAACGCTGTGGCCTTGCCCCCAAGTCATCCCTGATTGGTGAATAAGATGCCTA
CAGCCTGTAGCTGGACAGGATTGAGATGGGCAGGGCTCGGGGTTCCCAAGCTTGGGGTTGAGAAAAGGGAGGAGGAGGG
AAGAAGGAAGGAGCCATGAATTAGGTGAGTCAGTCCTAAAAGGACAACCATGAGGACTGGCAGTGAAGTTACGGGCAGC
CCAGATGGAGCATGGCAAGTGATAACTCCGGGTTACTAATGGGGAAATAGATTCTAGTAGTGTAGAGGATAGATAGCTG
CCCAACTCTAGTGCTGATTAAGTCTTGTTATGATGATAAAAGTTTCGTGTATTTTATCTGGGAACTGAATGATCAAAGG
TGGGGTAGAAACCCTGACTTGAGATTAAATAATTACAGCAATATTTTATTGCTAAATTCCTTTGGCATAACAATAGTAC
TTGGTTTTCTTATAGGTCTGTGGTCTATTTAAGCTAGGTTCTTGGCCACCCAAGCAGTGTCAGGCATGGGTTTTGTCTC
ATGGAGTGGGCCTTAAATCCTGTCACTAGAGGTGTCACTTGGTGGAGAGATGTCTAGTTGGAGGATTCTCTCCTCCATT
ATTTGGTGACTCCATTTAGATTTCTTTCATATATGTATGTATTTTAAGAAATCTCTGTTGTAGGTTTCCATGTGACTCC
TCAAATGGCCCTCCTTCATTTTGTTTTGATCATTGTGAGTCCAGTACTGGACATCTTAACATAATTCTCTAAATTTAGA
ATACCAACTCCCAGTTCAACAAAGTTCTCCCTCATTACAGCCCATGCTGGTCTTGGGCATGAAATCTTGCCTCAGCCTT
CTAAGTGCTAGGATTACAGGCATGTGTTTTGTCTTATTCCAAGTTGAATTGAGCTTAGCATATTATTGTAAGAATGTCC
CTTCATTAAATTTAACGTGGCTAACTCCTAAAAATGATCAGATTTTTCCCTTAAATTCTTCCAGACGCATTTCATATGC
ATAAATACTCCCTATAAGTATCTTTTGCCTTGAATACTTGGGACCAAAAGAAATATTTATGTCTTTTCCTGTGACTATG
ATAGGCTGTTAGTTTAAAATTCTGCCATCAAGTTGGGGCATTGCATATTGTTTTAAATTCTTAAAGGTTATTGTTACAT
TCTAACATTGGGGTTAAAAATGCTAACATGTTGAACACATGAACACATGTTGACTTTGCTCTCTTTGGGTGGAGGTGGG
ATTCGTATTCATTCTCTCTTCTCTCTCTCTTTTCTCTCTTTTCTCTCTCTCTTCTCTCTCTCTCTCTCTTTTTCTCTCC
CTCTCTCTCTCTCTCTCTCTCTCTCTCTCTCCCTCTCCCTCTCTCTCTCTCTCTCTCTCTCTCTCTCTCTCTCTCTCTC
TCTCTTCTAGGCACAGTTTCTCTGTTTAACCCTGGCTGTCCTTGAACTTGCCCTGTAGACCAGGCTGGTCTTGAACTTA
AGGAGCCAAGCACCTCTATCTCCTGAGCGCTAGGAGGGGTTGTAAAGGTTGTACACTGAGGTTGAAACTCAGTGTCATT
TCTTAAGGGTGCTGAACAGTTTTCACCATCAGGTACTAAGAACTGTAACCCTGCTCCCACTGAATCTGCACATGGGAAG
TGCACACTTGCAGGTGTGTAAGTGCATACTTGCAGGTGTAGCTCAGGACCCTTTTTTCTCCTTGAAAGAACCTTATAGT
TCTGCAGTTTTGTAGTTAAGGAGTCTTCATGATTTGCTTAGAAACACACTGGTTGGTTGTTTTGTCATAATAGTAACGC
ATTGCCACTTTGGGCTTTGTTTTTGTAATTCTATCTGGAAACATCCAATATGTTGGTACATTAAGATTCTAATAACGTT
AACATCAGCTTTTAACTGTCAGCTTAGGAAGTACACACAACCAGATCCAGCTAGTTACGAATGTCTAGTATGCCAAGAT
GGGACCTCACATAGGCACTCTGAAGACGTCTGGGTATTGTTCTTGTTCTATGTTTTTGCCAAATGTAGGCACAACTTCA
GATGTCTGTTACGACTTTTTTCCCTTTATTTGAAAAAATACTTTTTGATTGATGCCTCCTTTGAATTATTTAGATAATT
ATAGTAGCTAACACAGTTCAGCTGTCAAGAGTCTGCCACCGATTGAGTGGTGAATGCAATCAGGAAAGCACAGTTGAGA
GTTTCATGTTGTTTTTCCTCGTCTTTGTGGTACTACATTGTGTTTAAAAAGCATTTTCTTGGGAAAAACAAATCAAAGA
AATGACTTCCATGCTAATCTTATCAGCATGGTTTATGGCACTCGGGGGATCCTTTTTAAGGCATGTGAATAGTTACTAG
GCCATAGAGCAAATCAAATGCCCCCCATGTGCCTGCTGTTTCTTTCTAGACACCGGTGGACATTGTCCTGGGAGCAGAG
TCTGCAGTCATTCAGAGCCTGCATCTGCAGCATGCTCCATCTTGCTCCAGTCCGCCAAGTGCTCTAGCGGCCATCGCCA
GATACTGAGTAGGCTCAGTCAGGAGACTCATGTACAGCGGGTGGTGTCTAAGGGAAAGCTGGAGTTTCTGAAAGACGCT
TCTTATTTTCAAAAGCAGAGAACTCTTTCTGTCTCATATTTTCCATTACTAATTTTAGGTAGGGCTTAGAAGATCCTCA
GTAGTTAGTTGTGTGTTTCCTTCATGGTTGCAGATTTTATACACCAACAATCTTAGACCTATTATTTCAGTATCAAATT
CACATTAAGAGAATTAGTGTAGGGACAGAAGAACAATGTAGACATGAAAGGATCAGTTTAGACCATGGTTTAGGAAATA
GAGATTTAAGATTTAATGTTCTTGATGTTAGGGTCCTGCTTCATTGTATTGCTCTAGCGTTGGTTGTCCTGGCGTTTCT
TAAGAACGAGCAGTACAGGGATCACTCTAAGAACAGTTGCTGTATTTCCAGACGTGGTCGACTCCCCTGGCAGTGTTGT
CTGCAGTACTGCTCTCTCAGGATCTCACGCGTCTCTGAGCATCTCTCCTCTGGAGCGCTTTGGAGACGTGCTCCTACTC
TCTGCTCCTACTGTCTCCCTTAGAAGACCCCAGGATTCCGATGAAGCCCTGTGCGAGTGTCTGCTGGCGGAGAAAGTGG
CGTGAGCAGCCAGTTCTGTGATAGCCGTCGGTTGCGTGTAGGTCTGCAGGTGTCTAAGGATTGGTCACACCAGCAGCTG
CAGATGCTGGCTTTTGAGGAAGGCGGATAGACCCAGTAGCTCAGGACACAGGCTGCATTCCTCCAGCTCTGCAGCCTGG
GGAGTCCAGCATGAGGGCACTGATCAGTGTCCTGCTTTCTGCTATTTCTTCACATGGCAGAGGGAAGAGTGGGGTTTGC
TGAGGTCTTTATAAGACTTTATAAGCTCCCATTCCTGGGAGCTCCACCTTTATGGTGTTAACTCCTCCCATAGCCCCAC
CCGCTAGCTTTATCACACTGGGCATCGGGAGTTCAAAATGTACACATGGTGGAGGCATGGTGGAGTCTCTCAAGTTCTG
CTGCTGGGCCTCCTAATTCACACCCCTCATGTAGTAGATCGCTTTCATTCCTTCCTGACGTTCCTTCCTCATTTGCCTA
AAATCAGTCTCATCAGATGTGAGTGAGCCTCAAGGTATGATTTATACTCAGGTGAATTTGTTTTCAGCTCTAAGTTTAG
GAAAGGAAGCAAGTTACCGACTTCCAGAAACGCAGCGGTGGGACGGGTTCAAGTTGGTGCATCTGTCTCCAAAAGCGTA
AAACAGGAAGGGAAAGAAACCCAACAAACACAGTGCACCTTAGACTCAAGCAGGCCTCTAGCTCAGTGCCTTGCCATCC
AGGACCCTGGTCCCTTCATCAACAGCTGCCCATGAAAGTTCATTTGTGGCTCTCCACTGGGGTCCCAGCCAGGCAGGGC
AGAGCATGAGAGTATCCCGTCACACGTTTCCAGTGAGTGCTAAGGTTTCGTCTCAGTTAACTGGCTGGGGCGATTGCTC
TGCTGAAGCCTGTCTGGCATTTTAAATTCCCAGAGGACTCACACAGAAGCTGCCCCATTCAGTGACCTCACCCTCATAC
GTTAAGATGAAAACAATTGTGTACTTTCTGACAAACCATGCCAGTATATATTGTTCATTAAAAAAAAAAAAAAAAACCT
AGTTTTTGTCCTTCATTTTAAATAACAAGGTCAGAAGCTATGTCTCTTGGCGCATGTCTGTAGTCTCAGCACTTTAGGA
AGTTGGGGAAGGGAAGTTGTTGGAGTCTCAGGCCAGCCTGGGTTACAGAGAGAGACCTGCCCCCACCAGTGCCACCACC
ACCACTCGCAAAATAGAGGCTAGCATTCATCTGCACCTGTTACTTATACCTCCTTCTGTCAAAGATGGCCTGATGCTCA
CTGGTGAAAGGACAGCAGATTCTGAAAGGCTTTGCTATTCTGGCAATGCTGCCCCTGAGCACATGGATAGTGGGGGAAC
TGTGTTCAACCCCCACCCCCATTCTCCAAATGACAGGTGGGAAATTCTAACCCCTGAAACTAAGACTATAACTTTAAGA
ACACCTAGGACTTCCAGTGACCAGTAGAAGCTGGAATTAGGAAGGAAGGGATCACCTTTTAGAGCAATATTTAATGTCA
TTCCATTATGGATTTTTTGTTTTGTTTTGTTTTATAGATTCTGTTAAAAATGTGGCTATTGTTCACTAATATAGGTTGA
ATAGCCTTCGTCCAAAATGCTTGGGTTTTTTTCCCTAGACTTAATACATACATACATACATACATACATACATACATAT
GCTGTATAAATGGGACTCAAATCTAAACACAGGTCTGTTTTGTGTAATATTTTTAATGATTTTGTGCATGAAACAGTTT
CATGGCATGGAAATTTCTTTATTGTAGTGTCAGCTCTATAACTTAGATTTTGGTGTGTTTTGCATTAGGGATATTCAAC
CTGTGAAAGGCTTTTTTAAAAACAATCTGTGTATTTTACACAGTGTCTCCAGATTTTGTTCGTACTGAAACAGTAACCT
CCAGCTTTGTGCTCTGGGTGTGGATGTGCTGTGTGCCCCACCCAGCTCTGCCCTTTCACTCACTGAGATGTGTGCTTGG
CCAGCCATAGGTTTCACCTGCTTCCTCGCCTGGTAACCTAAGCTGCCAGCGTGAGGTTCAAGGGTTTGTTGTGAAAACT
GAATGTGATTGGAGGCTCATCGCAAAGCACTGGTGAGAACCCTTTAGCTCCTACACAGGTGCTGCCCCCTCTCCCCAGC
GTTGACGTGAACTCAGGTTCACAGGTGGCAGCAGGTTGTCCCCACCGTGCCCTGGACAGCAGCATAGCCGCAGCTCTGG
GTGGTGGTGGAGCCTGCCTTCCACTGCTCGTTCTTCTCATGAAGGGAAGGCCCGAGTCAGCCGGCCGGGCCTTGCAGTG
CCTCTGCTTGGGCTCTGCGCTTACTGTTCATCCCCTTACTGGTTTTAGGTAACTCTGACTTTTACTCAGCTGGACAGTA
TCTTTTCAGGGTGTTCCTAAAAGTTGTAAATGGTGACAACAACACATTGTCTCTCTGTGCATGCTAGGGTTCCATCCCT
CGCTCTGCAAAATAAGCAGATCAGAAACCCATGACTGCTTCTATGTTATGTAATCTGCATGTCTCGCTTACTGAGGGAA
TCTTCTGTCCATCTAGGTTGTTCTCCATTTCCCACCGTAATTCGTTAACTTAGGGGTAGCATGGTGAATAGCCAGTGAT
AGAAGTGGTTTCTTTGAACGGACCTAGTTACTTAGACCAAATGATACAAATGTTAACATTGAAGAATTAATGCATTTTA
ATGCTTTCTTTCCTACACCAGCTGGAGAAACTAGCCAGTATGGTAGCTTTTGTTTTATGAGCAGTACTCACTTTATTTA
GGAGTTTGTTCAGTTGCTATTTGTGACCTGTCTGTAGTCACCAGCGCCCAGTTTTAGAATGTTCCCCTCATTGTGCTGC
TGTCCTCTCCTAGTGGGCCTTTGGAGTGACGCTGTGGGAGCTCATGACTCTGGGCCAGACGCCCTACGTGGACATCGAC
CCCTTTGAGATGGCCGCTTACCTGAAAGATGGTTACCGAATAGCCCAGCCAATCAACTGCCCTGATGAACTGTGAGTTT
TTTGGATCTCAGACATGAGCCTTGGTGCATTTACTATGTTACTATAAAGCAGGAAAGCACGTAAGATACTCCTTCAGAG
TAAAGAAAAAAGTAGAGGACAGAACTCAGTGCTGACCTCTGCTCTCCACAGACATGCACAGACACATGCATACAACCAC
ACAGACATGCACATAGACTACATGACAGTAAACACAGACAGAGACAGACACCTTTCTCATCCTGGCCTTGCCTTCCTTC
CTTGCCTCCATCATGGCGGTGGGGTACACAGGGCCTGGTTGGGATGGTGGCACAGCAATGTGTTGTCTACAGAGGCCCT
AAAAGGTTTCCCTGCCAGTTTTAGATAGTAGCTTTGCTGTAGATCCAGGAGTCTTAATTTCAGGTAACACTTCTTCCTA
GTTCTTCCTCAACAGCGTCCAGGACAACGCAGCCTGTTATTCTGACTGTTCTGTGTATTTACACAAAGCTCTGAACTTT
CTGAGTATTGTATTCTCATAAAGAACTGAAGATGGTTGTGTGTAATCCAAAGAACTTAACTTCCTTTAATCCTGAAGCT
GCTAACAGTACAGCTAAGCGATTGCTCATGGCTGTCCTCCCCAGCAGCCCATTAAACAGAGCACATAACTGGCAGGACG
CTGACGGAGGCCTGACACGTTTCCATGTCACTGCAGGTTTGCTGTGATGGCCTGTTGCTGGGCCTTGGACCCTGAGGAG
AGGCCTAAGTTCCAGCAGCTGGTCCAGTGCCTCACAGAGTTCCACGCTGCCCTGGGAGCCTACGTCTGACTTCTCTCCC
AGCTCCGCCACTCAGAAGAAAGTGCCTGTCTGTCACGGATGCCCCTCGTGCAGCGCAGTGCCTGCAGGGGCACACTGTC
TCCAGATCACCCAGCCTTAGCAGTGCTTCCAAACCTCAGCTTTTAACGATGACGTAATAACGCAGAGTGTTTTCTAGAT
ACCACTTTTACTAGGTTGAACCAAAAGGGGTTTTGTACATTTTTTGGCCAAAAATTTTTTTAAAATATAGTGACTTTGG
ACTAGGGGGTGCATTCTTTTAAAAAATGAATAAACAGTTTTAAAAATTATTTAGACACAGATATTTGGAATAGCTGTCT
TAGTGCCAACTGCTTTTATTCTTAATGTTTTACTTCATTGAAGTAATGTAGGTGACTCACCTTTAAAGCTATTTTAATA
TTTTGGTCACTACTTTTGGGAGTGGTTTGTCTTCAAAATGCGGTACTTTTCTTAGGAAAAACAACATTACGAAGTTGTC
TGGTTTGCCTTATACGAAGAAAGATAGTATATTAGGAAAATTGAAAAAGTAAAAAGAAAAAGAAATTACAAAGTAAAAA
TAAAAAAAAAAAAACATATCGCCCCCGCATGGAAAATCGCTTGTTTAGTGACCATGGCTCTGCTCACATTTGGGGAAGC
AGGTCTGAGTTCACATCTCAGTCAGCTTTCCTGTGAGCTGGAAGAAGGCTGGGGCAGGCTTGTGGAAAACAGTGGTGAC
CTTATTTATTTTTGCTCTCCGGGGAAGGAGAGGACACAGTTCCTGACAGTGACCCCTCTCTACTGCTGCGGTTCCCTCT
GTGCACGCCTGGGCGGAGCTACGCACAGAGCCGGTGCTTCTGTTGTTGTGGGTTTGGGGGGTTTTAGTTTTGGTTTTTG
GTTTAAATTGGTAATTTTTATGCTTATCTTCAAGGCTGGCTTAAGTATAAATTTGTTTTTTTAAAACACTTGAAAAATT
AAAGGACTCATTTTATATGCTGGTATTCAAAATACTGCTCACAGTGTGTGACTGTTGGTCGTGTCCTGAAAGTCTTGGA
GTCACATTTTATCCCCTGAGAGTTTGACTCAGGTTCCAAAACCCTAACAGAAGAACAGCTGACTCTGCTCAGCCGCCAG
GGCCTGTTAAGAAGTTGGAGTTACTCCTACAAGGACTTATCATGGAGAGAGTCCACAAATACATGTATGTAGTTACACA
CAGGTTTGGTCTACCTAAGGGATTGTCACCTGGGCCTGTGAAGTGACTGGGCCTGCAGCTACTGCCTCTGTGTCTGTCC
TCTCAGGGATGTGAGGGAGAACATGGGTCTGTTCTATTAAAATAAGTCTTGTGAATAAAGAGAGTATCTGAAAAGACAG
CTTCCACAGTCCAAGGCTCCAGGAGGCAAGCCAGGAGCTCAGTGTCCCGCATAGGGCGTCCTGCATATGGTGTCTTGTT
TATGATATGCTTTGTCCTCAAATGTCTGGGTTTTAAAATGCCTCCTTTGAACCTTTTGTAACTTAAGCCACTTTGTTAA
TAGCCTGCATCTTAGAAGGGATTCAGTCAATGAGTGCTAGTGCGTACATTACACACTAGTACTTCAAGTCAGCATCCAG
TCAGCCAGTGTGTACATTACCTACACAGAGCAGTTCAGCTTCCCTCACCCCCCACTTCCAACACTCAGTGCACTCTTAT
TGTTTCAGATGAGAACAATGTTTAGAAATCACAGTTCTCTGTATAGAAGTTGTTTTAAAAGTCATTGTTCTGGGGCTGG
AAAGATGGCTCCATGGCTCAGACTGCTCACAGCCCTTCTAGGGCACCTGCGTTTGGATCCCTCTGGCCTCCACAGGCAC
CAGGCATGTACATGGTGTCATAAACTCATGAAGGCACATACTCATATACATACGTATGTATGTACCCATATATATAACA
TGTGAGTATGTATACCCATATATAACATATCTGAGTATATATGTATATTCATAGATCTAGACAGAACTGTTTTAAGGAA
AAGTGTGGCTGTCTGACTTGTGTCCCGGTTAAGTCGAACTTCTGTTTCCTTTGGGGGTTTGCTACCTGTGCACATGTGC
ATGCTCTCTCGTTTCAGTAGATTGTTCTACTGAACTATGTGGAGATAGTGACAAGGGAAGAAAGGAAGAGTCCGTTTTC
CACGGCTGATCCTCCTTCAGTGATTTGTATAGCTTGTGAATACCGCCGAACACCGCCCCCCACATCACTAAAAATGTAG
GTTTTTAAGAAGCAAATTGATGGAGTGCAGGATGCGACCATAAGCTGTCTCTGCTGACAACACTAAAAAGTTTTTTGAT
ACAGTACCTTGCTGTGTAACCCAGGCTAAGCTCAAACAAGTGCTAGGATAGTAAACACCACCATGTGTGGCTAAAACTA
AGATTTGTTTCTAAAGTAGTAAGGACAGCTCCAGGATTTAAAACAAAAGAAACAAACAAAACAAAACAAAAAACACAGG
TTGAAAGTCATCATCATCAAACAAAAACCTAAGATTAATACCATGGTGCCTTGAAGTTACATTTGGAATCAATGTTTCC
AGTTATTTTTATATTTTGGCCTTCTGCTTTGTTGCCACCAAGAATTAGCACTAATACTAGATAGGGGCTTACATCGTAG
TGATGGTAACTAAAAACTATTCACTCCACAGATGCTGATGGCCCTGAAGATAGATGGCTCCTCTCTAGGTCTGTCTGTC
TGTCTGTCTGTCTGTCTGTCTGTCTCTCTCTCTCTCTCTCTCTCTCTCTGTGTGTGTGTGTGTGTGTGTGTGTAAAAGC
ATGAGGTGGATGTTACATTTCTTCCTATCTCTCCATCTTAGTTTTTGTCTATCTATCTATCTATCTATCTATCTATCTA
TCTATCTATCTACCTACCTACCTACCTACCTACCTACCTTTGGCTTTTCAAGATAAGGTTTCTCTGTGTAGCGCTGTCC
TGGAACTCACTTTGTAGACCAGTTTGGCCTCGAACTCAGAAAACTGCCTGCTCTACAGTGAGTCCTGGGATTTAAGACA
TGTGCCTTAGTTTCTTAAGACAGAGTGTCTCATTGACCCTAGTGTCTCATTGAAACTTACCAGTTTGCTAGGCAGGCCC
CGGGGATCCAGCGGTTTGTGCTTCCCCAGTCCTGGAAGCCATCACACTGCTTTTCTACTTTGGTGGGTGCTAGGGATAG
AATTTAGAACCTCTGGCCTGGTAAGGTGGCTCAGTTTCTGAAAGGGCTTCCCATTAAACCTGATGACTGGAGTTCAATC
CCTAGGAAAGAGAAAACTGCCTCTTGCAAGTTGTCCTCTGACCTCCCGTGTGTCGTAGCATGCATGCCTGTGAACACAA
ACAGATGAATATCATAAAGAGGATTATGACAATAACTCAGTTCTCTGTCCTTGCATGACAAGGACTTTACTACTTGAGC
CATCTTCCCAGCCCCATGTGCTGTGATAGGAAAATGGGAACACCAGTTAGCTGAGTCCTTGTGTGAGGACGCCACTAAC
GTTCTTGTAGGATGTTTCTGACAGCATCTTGCTGGAACTATATCACATGGGCACGCCTGGCTATTAAACATTCTAGGAA
AAAAACATTTCAAGAGTTATTTTATGTGTATGGGTGTTTGCCTGTGTGTATGTATGTATGTATGTGCACAGTGTGTGTG
TCTGGTACCTGTGGAGGCCAGAAGAGGGTGTCAGATCTCCCTGAAACTGGAGTTACAGATAGTTGTAAGCCATCTTGTG
GATGCTAAGAATCAAACCTGGTTTCTTTGCTAGAGCATTGAGTGCTTTTAGTTACTGAGCCATCTTTCCAGCCCAGAAG
TTGTATTTTGTTAGGTTTTTATTTAAAAGTGGCTTTACTGAGATATAATTAACATTGCACCATAAAATAGATGGGTTTT
GATATATTCAGTGTTGAGTGGTCATAACCATAGTCGAATTCAATTTGTTTCATTTTGGGTTTTGTCATATGGTTTTCTT
TTTGAGGCAGGACCTCACTGTGTACTCCAGGAGGTTCTCAGACTGTGGCCCAGGCTGCTCTTGCCTGGCAGCCTTCTTG
CTTTAGCCTGCAAATGCTGGAAGAAAGGGGGACACCACTGTGCTAGCTCCCCTGTTGAGATGTGTGGCCCCTAGAAAAA
GCCCAATGCCCATTTGTGGTCACCCCGCTTCCTTTCTCACTCTCAGCTTCAGGCAGTAACTGATCTCCTGATCTCTATT
CGGAACTTTGCATTACATGGAGCCATGTGGTAAGTAGTCTTTTGTGTCTGATGTCTTCTACTTAGCACATGGTCTTCAG
GGTTTCCATGTTGTGACATGGCCAGCAAATGCTTCGTTCTGCTGAAAAGTATTTCCTTGTAAGGATATGCCACATTCTC
AGTTGACACCTGCTTGGGGACACTTGAGTTTTCGTTTAATCAGATCGTCACCTGGAAACAAAAAACAAAAAGAAGGCTT
TCCGTGGTAAGGATGGGGAATGGCTGTAGGTGGCTCCTAGCAGGGTCAGCCACAGTGTAGAGATGGACTCTTGACCCAG
CTTCCTTCAGCACTGGGTTTCGCACTCCACGAGTTGTCAGACATGAAGGGAAGCAGGGTGAGTCTAGGAAGATTCAAGT
TAGCAGAGACCACTACTGAGGAGAAAACTTGGTCGGAGCCACTATCTGAGCTTTTAACTTCTGGCTCCTGGTCCTGAAA
GCCCTACAGTACATCATGGCTGCTGCCACTTTGACCTCTGCTCTTTTTGCACCAACCTGTCCTTGAGCTTGCAAAGTGA
AGTATGTTCTCTGTACTTGGTACTATAAAGTCTATCTCAGGGTGCTTTCGCTTTGGCTCTGATCTAAACCACCTAGGAC
CTTTGGCCTCTGGAAATAATGTAGAAATCAGCCGTATGCCTCTGCAAAAATTGGAGGTAAATGGTCTTAAGCTTGTGTC
TAGTGATACAAATCTTGGACTAGGTCTTGAGTCTTTATCTGCATGTATAAGAGGTTTGTTAACGTTTTCTTTTGGAGAC
CAAATCTCACGGTATAGCCTAAACTCTTATCTCCTACCTCCCCTCCACGAGTGTTTCAGCCTCCTGGGCAGTGGAATTA
CAGATGTACAGTACCACGCCCAGCTCGACATTTAATTTTAAATTAACTTTGTGTGTTGTGTGTGTTTGTGCACACACCA
GGCGTCAACATCAGATGTCTTCCTCGGTGGCTTTCCATTACATCATTTTTTGAGGCAGATTCTCTTCCTGAACTGAAGT
TCAGTCTAGTCCGATTGACTGGGAAGTGGACTCTCCTGTCTCTCCCTCTCCAGCCACACTTGACTTTTCTGGATGTGCT
GGGAATCGAACTCAGATCCTGGTGCTTCTGTGACCAGCGGGCTCGGGGGCGTCGGTCAGTGTTTCTCTGGCTTCACAGA
ATCACCAGCGTTACTATTATGAGGAAAGATCCTTTGGCTCCATCTTGTCTGGAGCCAGAATGGCCTTGGCCTGACTGGG
TCAGGAAGCAGAGTGCGTAGTACGAGTGTTTTAAGTTTTGTGTTGGAGTCTCTAGGCAGTGTCTCTCTGGCCTTGGCTC
CTCTTTTCCCCTCAGGACCCACAGTTGCCTTCACTGAAGGAGAGGGTTGTGTATTCTGGTCACCTAAACAGACTCTCAG
TCTGTGATAAATGCTCCCTTATGGTCCTCCTCACCTCTTCTGGGAAGGCAGGAAAAGGAAGAGACGGGCCTGGCCTGCT
GGACTTGGTGGGAAGCTCCCCGCGCCTCCTCCGCTGGCCACTCAGGTCTCTGCACGCTCTAGGCTGCCCCACTGGCTCA
CGCTTTGCATGTCCCTATTGAAGGAACACTGATATCTTTCCACAGGATGCGGCCCTTAGGTGCATTAAAACAACATAAG
ATCGGGAAGTAGATAGAAATACTGGATTTTCCCATCACCGCTTCAGTGTAGTGCTGTGTTGTAGAGATGGCCAGCTGAT
AATGTAACTCCCTTGTTTTACAAATGAAGGAAATGAAAGGCGCTTTTGCTTGCAAACCTAAGATGTATAGCAGGCTGTG
TCTTATGTTGGTGTATTATAGGAAGTGGGTGTGCTTTTCAGGAAGGGAAATTAATTTGGTAGGATGCTGACTAACACTC
TGTTAATAGGATTTTTTGTTTGTTTGTTTGTTTCCTGAATCCATGAACAGAGTGCTATTATTGGCCAAAACCCTTCAAA
AATGATGTTGCTGGGAGGGAAGGTTCCAAGGTAAATGGGCAGAAGTGCAGTACGGGTCTAACGTCAAGAAATTCCAACT
TTGGTCTGGAGTAATAGAAACTCAGTAAAAGGCTCACATCAGAGATACACCCACATGTGTTTTAGCAGTGTTTGTGGTA
GGCAGATGTTGCCACCGTACATCAAGCATGGTACTTGTACTGGCTAGTTTTGTGTCAACTTGACACAGCTGGAGTTATC
ACAGAGAAAGGAGCTTCAGTTGAGGAAATGCCTCCATGAGATCCAACTGTAAGGCATTTTCTCAATTAGTGATCAAGGG
GGAAAGGCCCCTTGTGGGTGGGACCATCTCTGGGCTGGTAGTCTTGGTTCTATAAGAAAGCAGGCTGAGCAAGCCAGGT
GAGGCAAGCCAGTAAAGAACGTCCCTCCATGGCCTCTGCATCAGCTCCTGCTTCCTGACCTGCTTGAGTTCCAGTCCTG
GCTTCCTTGGTGATGAACAGCAGTATGGAAGTGTAAGCTGAATAAACCCTTTCCTCCCCAACTTGTTTCTTGGTCCTGA
GGTTTGTGCAGGAATAGAAACCCTGACTAAGACAGTACTAATCACCAAAAGTCCTACAGAGTGGCCTTACCCCCATTTT
ACAATTGAGGAAATGAAAGTTGAGCTTAGTTGCGTTAAGTAATGCTGTGTCTTAATCCAGGGAGTCTGTCCCCAAACCC
TGTGCTACTGAGACGGGAGGAACGGGTTTGCAAGGGAGGCAGGACACCTTAGGAGCCCCTGCTTGTTGGTTCCAACATT
TCTAGTGATATAAAAGGTAGTGTTAGTGTTTGGAGTACCAGGTTGGGGGTGGGCTTCATAGGAACTTGAAGTGTTGTAG
AGGGAAATTAAAACGGGAGCTGCCTACAAGCAAATAAGAAGGGCACACAGCTAGTGTAAGTCACATTGGGGCTGCCCTG
GTTGGTCAGCACATTTTTCTTCAGAAATTTGGTTACAGTGGAGTCTGCTATAACAAATGGTAGATTAAGTCATTGTTTC
CTTCTTACATGGTAGTCCAAAGGGAGGAGGGTGGCTAAGAACCCGTCATCCTTTCGCCCTGCCCTTCCTATCAACGGCC
ATTTTAATGTGGCTGCATGGCCAGTGCTGGGTTCCAACCACATCACTGCGGCTCCCAGAAGAGGGAGCCTTTCTCAGTA
AGTGATAGAAAGGCCACCAAGACACTTCTGCTGATATCCATCGGGAAAGACTCGGGCACCAGCAGCAAGGACGTCATTT
AGAAAAGAGTCTCCCTAGGCAGCTGGCTGGGGAAAAAAAAAAAAAAACAGTTTGCAAACAGTGGTCTGTGGAAGAGAAA
TGTTGAGAGAAGAGCGAATCAAGAAGCTGTGTTTTTCCAGATAGGTATGAAGCTAGCAGCACTCTGGAGTGTAGGCTGA
TAGGGGAAAGGTGTTACCTGGCCATGGGCTCCAGTTAAGTATCTGGTCCAAGATACTGAAGATCTGAATACCTTGGGCC
TAAGTCTCCAGAGTAAATCTGGGACTGAGACAGAAAGTAGATGAATGAGAGCTGGTATGTGTGGCTAGAGGAGAATCTG
GATCTAAGCCCAGAGTGTCTTCGTTCCAGAAGATAAGCTAGCTCCCTTGCAGTCATGGTGTGGGAGGCAAGGGTTTGGA
GTGGAACAGGCACAGCATGGTGTGGTCAGAAGTGGCATCCTAGCACACAGCACCCTCTACTCTATGTTGGCTTGCTCTA
ACACAATACCTGAGGTGACTGAAGGAAAGAGAAGAGCATATGTTGGCTTACAGTTTTGTGGAGGTTTCAGTTCATACAG
GACCAACCCTCTTGCTTTTGAGCTAGTGGCAAGGCAGTATGGAATGGCAGGGCACACGTGGTGGACTAAATAGTTTCTT
TATGGGAGCCAGAATCCAAAGAGAAAGGAGCCGTGCAATGCTTACTTGAAAGGCATACCCACTTTTGACAGGAAGGCCT
CTAAGCAGTCCCCACCTCCTAAAGGCTCTACCCCCTTACAACTGGGACCAAGCTTTAACACATGGACCTCTGTGGGGAC
ATTAGCCCAAACGTTAGCATCATCTGGTCCCTGGCTTACACACATACACACACCCGCCTTGTGCCTTCACTTCTGATCT
CCTCTGTAATGTTGTTCCATACCTACTGCAGTCTAGCAAAGCTCCCTGTTCTATAAAACCCACCTCTCAGGTTCTTCAG
TATCCTTAGTCTCTGCTTGCAGTCTGACCACCATCAAATCCACACACACCCCCAACATCAGGTTATTTATAGCATATAA
GTGTCACATGAGCAGATGGGAGGGCATCTCACATTTAGACCACAAAAGCAGCTGGCCATGCTAGAGGCCCCTTGGCCCT
CTTAACACCAGGACTATCCAGTTGGCACCCCAACTCTGTCATTATCTTCCCCTCTCTCTTGCACTCTGACCCTGCTTCC
CACTCTCATCCTCCTCTCTAGCCGCTCCCAGCTTCCCTTGAACTCCATCCTAGTCAACAGCTCCTCCTTCTTGCTGGAC
AGAGTGTTCCTAGATATCCTCTCCGTTAGCACAGCCTCCCATGCTTCTCCTGGACCCCTCAGACTTGTATGCATGGTTT
TCTCTGCCAATGACTTTCTCAGCTTCCAGCACCCCTCCCTCCCTGCCTTCCTGTTAGCATTGAGTTACTACTTCTCAGT
GAGTTACCTCCACTCCACTCAGCGTCTGCCCTGACGTGGGCCCAGGGTCCTTCCTTCCTGGGGTAATGGCTCACTTTGC
AGGCCCCTGACACCATAGGTGCCAGGTTCAGTGACATTCTCTCCGTGGCATGTGTCAGCATATCGTCCTTGAACTGCCT
TCTGTAACTTTGGCTTTTCAAATCCTACTTTGAATAGTTGTTCTTAAATGCTCCAACCTCCCTTCTAGCCCACCACCCA
CCAGAGGTAATGGGAAAGAAAGCTTATGGTGATACAGGGGCAGTGGGCCTGTTTAGAAATTGTTCTTTGGAGCAAATCC
CATCTGTGTTGTCAGGAAACCAGCAGTTCAGTTCACAGGAATCAGCAGCGGCAGCTCGATCCACTCGCAAACACTTCAC
CAATATAGCAGCAGTCCAACCTAGCAGAGACAGCCAGGCAAGGCTTCACCAGTCAGCAGGAGGGAGCAAGACCAGCAGG
GACGCCAGGAGAAGTTCTTGGCTGTGCCTCTCCCAACGAAGCCAAGATCAGTGAAGACTTGAGACGAAGAAGGGTTGCA
CAGGAAGCCGCCAGTCACTGTCCATTGAGTCCTATGTATGCTCCCCCCAAACATCATGTGTCCTCCACAGGTCTTGCCT
CAGCACATGAGCCTGTCTTAGCAAAACTCCCCGTGGGTCTGTATCAGCTGACATCACTCTGCAGATCGGCCGTGGAGCC
CGTGGAAGCGGCAAGAAGCCACGGGAACCTCTCAAGTTCTTTGGTGCATCTCTCTCTGTGGAGTCAAGACAAATGGTGC
TCAACAATGCATGTAAGGCAGGCCAATACATGCATGTCCTTAGCGAAGAATTCTTCATCACACATCCTTTCATGTGCTT
GCTTCAGCAAAATGTCCTTTCACCTGTGTCTGCTTCAGCGAAACATTCTTTGAAATAACTGACTTTCCAAAGAAAACCA
CGTTTCCACTTCAGCCTTCCTTCCACACTTAGTAATTTGGGGCTCAAGCGTCTACCTCCCCCCACCCCAGCCCCCCAAT
TTTGGGTTCCTATGGTTCCTATGTCAGTCTTGAGCACCTGCCTGTCAGGTCTTTTCTGTGCTCAGCACCTCTGCAGGAC
CATCTGGCACCCCTCAACCCCAACATTGAACTTAGTCCCTCCTTAGTCTCCACAGGGCCACCAGGAAGCCACCCTTACT
GCTTCCTAGTGCTTCCTGAGCCATCTGTGGGCAGAACTTGTGGTCTCTTGCCGGTTCTCTCCAGACTCCATTGCCCCAG
CCAGCTTCCCTCCCCTACGCCCACATTGGTTATACATGGTTTTTGATTATCTGTTTTGATAATCACTCAGGCTCTGCAA
AATTGTTATGGGAGATAGTTTTGTTCAACCAGTGATACATAGGACCATGTGAACTAAGAGGGGTGTGTGTGTGTGTGTG
TGTGTGTGTGTGTGTCTGTAGGGAATATATGTGTGTGTGTGCTGTCTATTGTTGTCTTTCCTGAAACAAAAGGCTACTC
CACCCCCTCTGCAATTTCCTTTAGCCTAGAATTTATTTTTTTTAATTTATTTTTTAACTTATTTATTATTATAGCTGTC
TTCAGATACATCAGAAGAGGACATCAGATCTCATTATGGGTGGTTGTGAGCCACCATGTGGTTGCTGGGGTTTGAACTC
AGGACCTTCGGAAGAGCAGTCAGCGCTCTTAACCCCTGAGCCATCTCTCCAGCCCTAGCCTAGAATTTATTAAACATTA
CTTCTCACTGAGAAAATACACTTATGGAGTTCAATATTCTAAAAGTTTGTATTGGCAAAAAACAAAACAAAAACTAAAT
ATATTCCAAAGGCTTAGAGGAAGGTATCTAGACAGTTAGCAAACCTCCAAAAAAGAGAGATGGACTTTTATCCTTAATA
TTGACCTGCAGAGGGAGCCATGTATAGGGGTTTGCCTTTATTTTTTTATGAAAACCCTTCTGGTAGGAGCCTTTCCCAC
AGCCTGTCTTTATGGTACCACTCACACGCCGGCCCCATAATCACTCGGCCTACATACACAGCTAGTCCATAGAGCTGCT
TTCCTGGAGAAGTAGAAATGCACGCACATGACCTGATCTCTGCTTCCAGGCCTGAGAGCACAGGGCGCCTGCTGGGGGT
GGGCTGCAGCCTGTGGGCTGGGAGCCACTGAGGAAATGGCCTGTGACCTGATATCAGTCATGTCCTGCTTCATTGGCTC
TCTCAAAAAACATCACCTAGAATGCTTCCGAAGAGGTGACACTGCTGGCTGACCTTACCTGGGGCGGCCCTGAACCTAC
AACACGAGTTTTCTCTTTTCTTTTCTTTTCTTTTCTTTTCTTTTTTTCCCTCCCCTCCCCTCCCCTCCCCTCCTTTCTT
TTCTTTTCTTTTCTTTTCTTTTCTTTTTTTTCTTTTCTTTTCTTTTCTTTTCTTTTCTTTTCTTTTCTTTTCTTTTCTT
TTCTTTTCTTTTCTTTTCTTTTCTTGTTTTCCCAGACAGGGTTTCTCTGTATAGCCCTGGCTGTCCTGGAACTCACTCT
GTAGACCAGGCTGGCCTCGAACTCAGAAATCCGCCTGCCTCTGCCTCCCAAGTGCTGGGATTAAAGGCGTGTGCCACCA
CCACCCGGCACAACACGAGTTTTCAACAGTGGCCTGGCTCCTCCATACCTCAGAAATCAGGTTCCTTGTCACTTTTTAA
TTTAATTTTATTTTATTTTATTTTTTTGTAGTGAAATGAGTACAGGGTACCTTGTTACATAGTTAACAAATACACTTTC
TTACCAGGCACAGGCTACTAGGAAGTGCCTTTGAAATTGTTGACTGAAGTTCTACTGAAATACACTTGTGAACTTGCTA
CAATAAGCATTTGATTTCTTTCCTATCTGAGAGGAAATGTCAAACTCTGAATATAGGCTAGGGATTTATGGCTGGGTCA
ATAAAATCTTAAGAGTGAGGGAAAAAAACCCTATTTTTGAAGGCTCGTATGGCCTTTTCCTATTACAGCAACCCTGTCT
TTCAGGGTTATTTTAAAATACCATTCCGCATGTTAACTCTGCCTTCAGAGGGACAATAGTCAGCAGTCTGCCTCAGCTC
TGTCCTCCACTAACAGAGAATACACTTAGGTCTTACAATTCAGTTGTTGTTTCTTTAATAAAGGCTGCACTGAACTTAC
TTAGAGACAGAGCCTGTCTCATCAAGCAGCCCTGGCTGTCCTCGAACTCCCTTGGTTGACCAGGCTGGCTTTGAACTCA
CAGAGATCATCCTGCTTCTGCCTCCCACATGCTGGGGTTAAAGGTGTATGCTACCACCGTCCAGCCCCATAAATTGCTT
TTGTCAAGAGTATTTTATGACGGCAGAAAAAGAAACCTAGACACTGGCCACCCTCTATCCTTGCCCCGCCCCACCCCCA
CCCCCGACCAATAACTTAGGCCCAGGAAGGCTTGTTTTCATTTGTTTGTTCACTCCTGCTAGGTCAGGTTCCTTGGGTC
ACTTTCCTTGAGGTGCCTGGAATCTGCACCGTGCCTTGAAAACCATCTTGAAAGCCAGCCATAGTTATACATGTCTATA
AGCCCAGAACTCTAGAGGCAGAGGGAGGGAACCTAATAGAGGCCACCCCAAGCTACAAGGAAGCCTCATGTCTCACCAA
AACAAATAGAAAACACAAAAGGGAAAAAGGGCAGTAGGAGAGAAGAGGCTAACCCAGCCTGAATATTTCTGGGAATTCC
AGGCCTGGGTTAAGAGCATCCCTTTCAAATGAAGACCTCAAGCTTCCCATGGCCGTTAGCATCCTTTTCCAAGATCACT
GCCTCTCTGGTTAGCATCTGGAATGTCTCCACAAAATGTTCACGTGTTGAAGGTTCAGTCCACGGCTGGTGGTGCTGCC
TGGGAAGGTTCAGGAACTTCAGGAAGTGGCGTCTAGCTGGAGGAGACCAATCACTGGGCATGCCTTTGGGGAGTCTGTT
CACGTCTCCCTAGTCTGTCTGTCTGTCTGTCTGTCTCTCTCTCTCTCTCTCTCTCTGAGACAAGTTTTATTCTGCACCT
TAAGCTGTTCTGCAGTTTTCTGTATTACCCAGGCTGACCTCAAAACCTCCCAAGAAAGCCAGCATGCCTGGCTCCCTTC
TTCACTTTCAGCCTCTTGTCATTCTGGAGCCGTAGGCAACACCAGAAAGCATATCTTTGCTCTCTATGCTACTAGATGT
CTGGACCTATTGGTATGTTTTGTGCTGCTGTGGCATGGTATGGCAGGGGGGGGTGCTTTAGCGGGCCCATGATGAGGCA
TTTCTGCCCCCCCCCCCCAGGTACCAGCCATACGATGGGTCTAGTATGAAATGAAGTTTATTTGGGGCATGGGGGGGCG
GGGGAAGAAAAGGGACAGAGAGGGATGGAAAGGGACAGGAGAAAGAAGGGAAGAGGCTGACCAGGAACACATGGGGTGG
GGGAGGTAATGGGGTGGGGAGGGCAAACCAGGCTCCTACCTGACTTGCTAGGTAACTGTTGGGCAGAGCCTAGAAGAGA
TGCTAACACCCTCAGCCTGTGCTTTCACATCTGCCTCTCCTAGGGTGGGAAAGCCTGAGAAAGGCCAGTCTGGCCCTTG
TTCCTTCTTGCCTTCTCAAGGATTGTGTTTTCTGTCTATTAGATCTGTAGTCTCAAACCGGAGTGAACATCATAGCCAC
CCAGAAAGTCTGTTTAGACCCAGAGCCCTGAGCCTCACCCTCGAGGCTGGGATGGAGCCTGCCTGCTAGCCTTTCCATT
GTGCCAGGATGCTGCTGTGTTAGTCTGAAAACCACAGTGGCCACTGTGCAACAGCTGTCAGCTTTTCTAAAGTCTTGAG
AGAATTTTTTATAAACTCATCCAAGATACCCTGGCTACCTACCTAGAACACTTATATCAGAATTTTAGACATCATTATT
TATATAAATATATCAGTAATTTATAATATATATATATCCTACATAATTCCAATGTGTAGCCAAACTTGATAACTGATAT
AATAGTTCTTTATTCTTCAACTCTTGTCTATGAGAGACAGACAGACACAGAGAGAGAAAGAAAAGGAGAGACAAACAGA
CAGAGACAGGGAGAGAGACAGAGAGATTAAGTGAGCACAGCATGTGCGGGCATGTGTGTCCCATGGTGCATGTGTGGAG
GCCGGAGGACAACTGCTAGTATTTGGTTCTCTTTTTCCATTGAGGTGTCAGGGATCAAACTCAGGCTATCAGGACTAAG
AGGTAAGCACTTCTAATCAACTGAGCCATCCTTCCAGCCCACCAGTGTAACAATTCTAATCAAGTTCTTAGTTCAGGAT
TTGATCAGCTGGGAAGTGTACTACATCCATCCTTCCACATGCGGTATAGCTAAGGTAGGCTTCCTTTCCGAGGCTCATG
TTACAGTGTTTCTCACTGTTGCTCTAATTAAACAGGACCAAAAGCAACTAGGTCAAGAAAAGGTTTAGTTCTCCTTACA
ACTCTGATCACACTCCATCCCATGTCAGAGCAGGACCTGAGGCAGGAACCTGGAGGCAGGAACTGAAGCAGAGGCCATG
AAAGGTGTGCTTACTGGCTTGCTACCTAAGGCTTGCTTGTCCTGCTTTCTTATGGAACCCAGGAGTGACATCACCTGTG
ATGGGCTGGTCCCTCCTTCATCAATCATCAGCCAAGAAAATGCCCCACAGGCCAATGTAGTGGTTGGGGGGGGCGGTGT
TTTCTCAGTTTCCTCTTCGCAAATAACTCTAGCTTGTGTCAAGGTGACATGAGACTAGCCAGCACACTCATCCTGATGA
TGAGACATGATCAAGAAGTGATGGGGGTCGGGGTGATTTGCAGATGATTCCGTAATGAACCCAGAAAGTGGGGAAAAAA
ACAACCATGTATCAGAGTACATTTAAAATATTAATAACTAAGAGACCTGGCAAAGAGAATTTACCTAAGTGTCTTACTT
TCCAATAGATAATAATAGAATTAAAAACAAATAAACAAACAAACAACAACAACAAAAAACAAAACCGAGCAAAGATAGC
TTTTAAAACAGCCAGATAGAAATAAGGAATAAACAAGTGAATGAATGCGCAGCACATGCAGGGCTTAGCGAGCCAAACC
CAGCTCTGATTTCATGGTGATGCTGACTGTGTTTCTGATGGGGAGATCCAAGATCATAAGCACGTCACTCGTTCACGAG
TTTGCTAGTTACAACACGGACTTGGCAAAGGCCTTCTCACATTTATCAGGAAGAGAACCCGGTCAGTAAAACAGTGCAC
GTGGCAGTTCTTCTGTGTAGTGGTAACTTTGGCTAACCGCTCTGGCTCCTACACACACACACACACACACACACACACA
CACACACGTGCGTGCACCAGTTATTTTAAAATGCCTCGACTAGCTCAAAGGTTGGGCATTTCTAATCCTTCCCCCTGCT
AGGAAACACTTGGCTCTGGTACTCCTGAGTTAACATGTTGGGGAAGTGGCCCATGTGGCCTCTTTCCCTGATTCTCACA
TGTCGGCAGACTTTTAGGTCTGGTCTTCCTTCTTCCCCATCATGGCCATCTCCTCCTTTCTCCCCCACCCCGCTTGGTT
TCACCTCAGTCTACCTGTCCAGCCATTGGCTATATGGCAATTATTTATTGCCAATCAAATCTAGCTGAGGGCAGAGACC
CTCAGCTTCTGGGAAGAAGGCGGCTTTTGGGAGCATAATTGAATCGAGACAGCATTAGAACCAATCCCTAACACTTCTG
TCTTGGAGCTTAGGAAACTGAGGCACAGAGGGCACATTTCCCAAACACATCCGGGACATGACAACCCAGGAGAGAGAAG
CTGATAGAGGGTTAAGGTGGGGCACTGAGAGAGGAGATCTCCAGTTCACAGAGTTGAGAGTTTCTGTGATTGCATTGGG
TTGGTAGTCATTGTTACAAGATCCTTAAAACCAGAACTGCCCAGAGAAGTGATAGTGATTGTTATTCTCAAATGGTTGG
AAAGCCAGGGGAGCCAGCCTGCTGGAGATTGGCATCTGGGAACCAACCGGAATAAGAGCACTTGCTCTGTACCTCGTGT
TGCTTGTTATGGTAAACTGATTACAGGTAATAAAGATCCCTGAGAAGTACATGGTGCTCTGAGGTTGAAAAATGTGTTA
GCTGACCCATGTCAGCGTCAGAATGTCTGGGCAGCATTGCGGGTGGAGGGGACCCCGAGATCGGCAGGGCTGGTTTGCA
GAGAGCGCTCTGTTCTTCTGAGTTCACAGACTTACAAAAATAGCCAAGTGTTCTAAATGTGCTCCATCCCCACTTTACA
TAACTGTGCGACAATCCTTCAGAGCTGAGGAGTTTTAGTTGAGATAGTTTGGGAGTTTATGCAAGTGGAGGGCTCAGTG
CCTCCTAGCATTGATGGTCCAACCATAGCCTCAATGACAGCACAAGTCTGGATTCTTGAGTGCTGGCAATGGCTTCTGG
TCATGAGAGCCTGCTTTGCCGGAGGCCAGCCTGAGGCCTGAGAGGAGAGGTCATGGGATGAAGACTTCTCTTCCTCTCC
TGAGGGCTCCAAGATCTTAGTCAAATCCCATGACCATACCAAGTGTCCTTATTGAAAGACTGGCTGCACTGATCATCTG
GGTTATATTGAATTCAGTGTGATGGTGGAGCAGATGGTATTCACCAACTTTTTCCTGGGGAGTAGCTGGGGAGTAGCTG
GAGAACCTAGTGGGATGGACCTGTGGGCTTGGAGGTAGACCTCACCTGTTTGTCCTGTGGACTAAATCTATGCTGAGCA
TACAAAATGTTAGATGTATCCATGTAGCCTCCACCCCCAAATCTTTAGACAAAGGCCAAACCCAAAGTAAAATGTCTGC
TAGGCATAACTGGGCAGATCCCTGGTATACAGGCAGCCCAGCTCAGGGAGCTGAGAGAAGGGAGGATTGGATAGAGCAA
CATGCACACATGATGACTGGCTTTGTTACAACCCAGAAGACAGAACTGGCTGTGTCTCGACCTAAGAGACAGAGCAGGT
ACTCAGGTGGGCAGGCCAGCGGGACTTCCTGAGGCAGTGTGGCCTGACCTTGAAGGACATGGCAACATTCCCATCGGGA
ACCCCAGCCCTTTGCAGCGCAGCCTCTTGAGACAAGGCCTTCGGCGACAGTGAGAAGAAAGTTGCCACATCTGCCACAC
CTTAGCCCTAGAACGAAGCAGTGCTGTTGTTTCTGTTTCGTTCGGCTGCAACCTGTGCTTAAGATAGCCTGGCTCTGCG
GGGCCCGGGAGGGCTGAGCTCAGTTCAGAGGAGCTGTGTGCTCCAATGGGAAGATTACCCAGAACACTGGCCTGGAAAG
ACAGTTCTCAGCACTCGCCCTCCCTTCTGTGGACACTCCCCACAGGCGTCAGTCTCAGACTGTCTTTCTCTAAGGCCCT
GGGGGCTATGAAGTCTGTGACCTGGATGTTGTGGGACCACAGCCTGATGTGCTGATTTTGTCCTGTGACAAGTACCTAA
GGACCCAGCGTAAAGTGTCTTCTTACCTCCCCAGATCTTCCTATAATCCTCTTGGCTGAGATAGCTGGGTTCTTGGCTG
CGGGGCACCATAGATTGGAATGCTGTGGACTATGTGGGGAGAGGCCCCTTAAGGAAACAGGTGCTGTTGATATCATCAA
CAGCTCGAGCAAACCAACACAGCCAGAAGGAGCTGTCTCAGGATGCCCGAGTTGGGGATATTCACAGAAGGAGAGAGGG
CGAGCTCTGGGGGACCTCTTCCCACATCCACTGTTGCTGGTGCGGGTTGTCTTGTGCCTGAAATTCCACTTTTGTGCTC
CTAAGACCTTGGCAGCCTCATTAACTTGCCAGTGCAACCTTGCTTACCTGTTTGGAATCCTGGGCTTAGTTCCCTGGCT
CTAGCAAAGTGTTATAGGACGCTGAGCTTCTTTGGCCGAGCCTTTGTGGCTTCCATGGTCCCGGCCACATTCTTTCCCT
CCACTGCTATCACCACCGTTACCAACCAAACTGCTACAGGGCCTGACAGGAAGCCAGTTTGAGATTAGGGAACAGATTG
GGGCTTCCCCACAGGCTGGTATTCCTTCAGGCAACTTCTTGTTGATCCTGGAGACTCCGGTAAACCTAATCAACATTCC
TGGGCCCCAGTGAGGCAGGCTATTGCTTCTGGCTATTTGCCTCATTGTCTCCTAGCATTGACGTCATCAACCTTGTGAT
CTTCCAGCTTGTTATCTCTAATAAGATAATAGTATTCTACCATCACTAGCCACTATCACTTCTACCAACTGTTGGTCCT
CTCAAGCCCACCCAGCTGCGTGACGAAAAACACCCTGCTACAGGACAGAAAGCCGCCGCTACCTCATCTGCTTCGTTCT
CCTCCTAGCCAATCTGAGAAGCCCAACTCTCTGGGGGGCGGGGCATGGAGCACAGCCCCCTTTTTTTTTTTGCCTTTCT
TACACGTGAGCCCTTAAAACTGGCCACGTGCCAGTCAGTCATCCTTGTCTCTGTCCAATCCAGAGGCCGCCCCCACCCC
ACCCCCACTGCCTTGTCCCTCCAAATAATAAAATCTCTGTGGTGAGAATCTGGCCCACAGTAGGAAGGGCTCCCCTATC
ACTTGATGTGATCATGGCAACCAAGGGCCCACTGGCGTGTCTGGAGGTCCATCTCCCAGGTGCCTCTGGATTGTCAGGT
TGACAACTAACACCAGCTACCACAATTACTTTATTTATTTCTGAGTTTGAGATTGTTTTGTTTTGTTTTGTTTTGTTGA
GACAGGGTCTCTCTATGTAGTCTTGGCTGCCCTGAACTGTCGATGTAGAGCAAGCTGACCTCAAACTCACAGAGACCCA
CCTCCCTCTGATTCTCAAGAGCCAGGATTAAAAGCATATATTACCACACCCAGCTGACTTCTTTTCTATATCTCACAAT
ATGAAACTTCTGTCAGGCAGATAATTTGCAAATGTTTTTCTCAATTTGTGGTTGCCTTTTTATTTATTTGATAATGTTC
TTTGAAGACAAAGTCTTTTAATTTTTAATTCACCGAATTATTGATCTACTTTATTTACTTATATTTATGTGATGTGTGT
AAGACAATGTTTCCTGACTTACTCCAATTGCTATGTCAGGATTGCTAAGCTTACTCCAGTGCTCCCGTCTAAGAATTTT
ACAGTTTAAGTTCAATTCTAAGTATTTTACAGATCTTAACACTTACACCATAATCTACTTAAACTAATTTTTACTTAAG
ACCTAAAGTGAAAACTCTAGGTCCTGTGCATATCTCATTATTTCCAACGCATTATTTGTTTAAAATAATTCTATTTTCC
TTTCTTGATTCTCTTGACTGAAAAACCGAGTGGATCCCATTTTGACTCTTGTGTTGATCCATATGCCCATCCTTTTGCC
AACACAGTTACTATCTTACTACCATATGTAAGTTTGCATGTGTGGGAGTGCACATGGTCTGGGAGGCTAGAGGTCAACC
CCTAGGGTGTTGTTCAGGGGCCATCAGTCTTGGGCTTTGGGGATATGGGCCTCTAACAAGCTAGAAACTCTTGGGATGG
CTAGAGTTAATTATCAACCTAACAGACTCTAGACTCTGCCCTCCCAGTGTTGGGGTAAAGAGCATGTGCCAGAACACTC
GAATCTTTAGGTCCCAAGGAATGAACTCACTTCCTCATGATCTGTAGGCAAGCGCTTTACCAACTGAGCCATCTCGATA
GCCCATAAAGTATCTTCTGAAACCAGAAAGCATGAGCCTCCTAAGCCTTCTTCAAGATTATCTGAGATATTCTGAGCCC
CTTGCATTTCTATGTGAATTTTAGAATCAATTTGCCAATTACTGAGTGGTGGGGAGACCCAACTAGGACTTAGCCTGTG
ATCACACTGAATCTGTAGATCTAGATGAGGTACTTGTACACCCTCATTCACTGTGAGAGCAATTGAGTGTAGCATATTT
AATTTATGTATGTAATTATGGAATGATCAAACAAAATTAATATTTGTAAGTATTATTTACATAGAAAAAAATCCAAATA
ATACAAAGAAAAAGTAGTAGAATTATTAAGAGTTTACCAAATTTGCTGGCTACAAGTTCAATATAAAAGGAAGCAATAC
CGTTTATATCAAGGGGGCTGTATACAAGGTCAACATACAAAAATCAATTATATCATATATACTAGTATTTTAGTTAGGG
TTTCTATTGCTATGATGAAAACACCATGACCAAGAAGCAAGTTGGAGAGGAAAGGGTTTATTCAATTTAAATTTCCAGA
TCATGTAGTCCATCACTGGAGGAAGTCAGGACAGGAACTCACACAGGGCAGGAACCTAGAGGCAGGATCTGATGCAGAG
GCCATGGAAGGGTGCTGTTTACTGGCTTGCTTCCCTGGCTTGCTCAGCCTGCTTTCTTATAGAACCCAAGACCACCAGC
CTAAGGATGGCACCACCCACAATGGACTGGGCCCTCTCCGATTGATCATGAATTGAGAAAATGCCTTACAGCTGGCCTC
AACTAAGGCTTCTTTCTCTTTGATAACTCTAGCTTGTGTGTCAAGTTAACACACAAAGTCAGCCAGTACAACTAGCAAT
AAACAATCTGTTTTGTTTTGATTTTTGAGACAGGGTATCTCTGTGTAGCCCTGGCTGACCTGGAACTCATTCTGTAGAC
CAGGTTAGCCTCTGCTTCCTGGATGCTGGGATTAAAGGCGTTCACCACCACCACCCAACTAGACAGACTCACACAAACA
CGTAACAATTCAATTTATAGTAAGGTCTCGCTGGCCTTTTCCTTTCTCATGGTCTCTCTGCTTCTGGGCTCACAGGAGG
CAAGCAGCTCTGCCTCTTTGAAGCCCTTTCTGCCGAGCTGTGTTTCCCCACAGACCCACAACAGTGGTACCAAACAATC
ACGGGTCCAAAGAAATCTTTCCTCTTTCTAAGTTGGTTTGCTCAGGGGGTTAGTGCCACAGTGATGGGGAAAAATGACT
AACGGAAGGAATGTATCCTGAACTTCTCTACCTGCACACCTGTTAATAGTATCATATAGTCTTTCTTTTAGCTTTTTAG
ATGGAGAACCAATTTACTGACTGGTGTCTTGGTAACACAGTAACATCCCAACCTTACATTCTGTGGGTAAATGCAACTT
TGTCCCCTTACATTATTTTTATGCTTTGTTGAGTCTGCTACAGTAGCATTTTTTGAAAGGATGCTTTTTGGGGGAGATT
ATGTTCCTGGGATAGTGGCCTCTAGTTTTCTCTCCTTATAATTTGTCAGGTCATGGTGTCCGGGTTATGGTATCACCTC
CGAAGGAAAAGTGTTTCTTCTTTTGGATATTGGAAATATTTTTTGTTTTTCTCTTTCTAGAAGAAGGCTAATAGGTACT
GGAATTGCTTATTTCTAGATTAATTAGTAGCTTAGATCTAGAGATTTCTTTATGGCAAGCCCTATGACTACTGATTAAT
TTCTCAGAGGATGTAGGACTACCCTAGCATTCTAGTTCTTCTTGCTAGGGTTTTTTTTTTTTAAATTGTTTGTTTGTTT
GTTTTGTTTCCTCTAAGAATTTCAAAGTTATATAGTTTTAATATATATTATGTTTTAATGTCTATGGCATGGTTATTTC
ATCTTTTTTTATTACTTTATTGTAGTTTTAAATTTTCTATTCTTCGTCGGGCATAGATGGTGCATGCCTTTAATCCCAG
CACTTGGGAGGCAGAGGCAGGTGGATTTCTGAGTTCGAGGCCAGCCTGGTCTACAAAGTGAGTTCCAGGACAGCCAGGG
CTACACAGAGAAACCCTGTCTCAAAAGAAAAAAAAATTCAATTCTTCAACAATGGATCTTAGCAAAATTGATCACTTTT
AAAAATTCTATCAAATAATAGTTTTTTGTCCTTCATATTGCATCTTATTTCTTTGTATTCATTTGAATTAATTTTATAT
CTTTTCCTAATCCAAAATATTTACCTTTCGTGTTTTTAAATATATTCATTGCAGTCATTGTCTTTTCTGTCACATGTTG
CTTTAGTTGAATCCCAAGACTTCGCTGTTCATTTTCATTGCTATTCAAAATAATGTCTGATTTCTAATGTGATTATTTA
ACATATTGGTTATTTATAAGTGAATTAATTAATCAATTACCAAATAAACAAAAATTTCAGTTATAGTTTATCAATTTCC
AGAATAATTACATAATGACTACAGAATGTTTTTTAGTCTCTATTTCTAGTCACAGAAAATTTGTTGTTTTTTTATGGTT
TTATGGTAAATTTTAGCAAGTATTCTAAGTGTGCTTGAAAAGAATGTGCATCAAAATGTGCCCCTTGCTACACATTTGC
TAATTGCATTGCTCAAATCAGCTCAGAATCTTGTTGATGACGGTATCCTAGAGCTGTTAGAGGTAGAGATGAGTTAAAA
ATAGTCTAAGCCAGCTTCTTCTTTCTTTCCTGTTTAAAAGAATAATTTATTGCTCTCTCTCCCTCTCTGTCTCTCTCCC
TTTCTGTCTCTCACCCTCTCTCACTCTGTCTCTCTGTCTCTGTCTCTATCACACTCTCTCACTCTGTCTCTCTCTGTCT
CTCTGTCTCTGTCTCTCTGTCTCAGTGTGTGTGTGTGTGTGTGTGTGTGTGTGTGTGTGTGTGTGTGTGCTACATATGT
GCAGGTGCCCATGGCAGCCAGGACAGGGTGTCAAGTCCCCTAGAACTGGAATTATAGGTAGTGGCAAGCCACTTTATAA
TGGGTGCTGGGAATCAAACCCAGGTCCTCTGGAAGAGCGGCAAGTGCTCTTAATCACTGAGCCACCTCTGCAGCCCCAG
CCAGCTTTCTTGTCATTGATACTTTCCTGATATAGTCACTTTCTGTGCCTGTGCATCCATTACTTTTAAGAAGTATCTT
GTTTGCTACAGTATGACACTTGTTAACCCGTTTCTACATAGCTCACATCTGAGTTCTCACTCCCTGCCCCAGTGCCCTG
TATCTATTTCCTTGCTCTTGTCTGGGCACGTTTTAATAAGCAGGATTTCAATTGTACACATGTGTGTTGTTAGTCATTC
TTCAGGCAATCATGATCGCTTTTGGTTTTCTGAAAATGAATATTAATCAGCATCTTCACCCACTCCCTGGGCAGTGGCT
CTGCAGCTCTCAGATCCATTTGTTCCTTTCCCATGGTTCTCAGGCGCAGTCTGTTCCACTGTGAGACATTTCCTTCACA
GCAGTTAGCGCACATGTGATTGATGAGGGGTGATGTCAGAATAATCCATAATGTCTATTAGTCTGGGCACGATTTGCCC
CAGTGCACTTGTGCATTGAAGCTGTAAGAGGCAAGCTTTCGTATTATTAAAAATGGAGTCTTAGTAATATGCGAAGGCC
TTTAGAGAAAAGGCAGAACCCCCTGTGTAAGTGTCCAGAGTCCATACAAAAAGGGTCATTAGGTGGCTTCCAGCGCCTC
TAGGAGGTTTGATAATCTACTCGGGAAGACCACAGTGTAAATGTAGAAACTGCAGAAATACTTCTTTATGAGTTATGAT
AATGTTTGCTTGATGGAGAAAACTACTTGAATGTACTTTTATTATTTATTTACCTTTTTTAACACGAGAGAATTTTACT
TCTCTTCTGTGTGTCATTTTATGAATTTCAACATAGCCAGAGGTTCAGGATCGTCTGTGCCATCAGGACACATAACATT
CAAAATGTCGTCCCATTCCCTCCATGTTGCTTTGATTTTTGATTAAACAAACAAACAGACAAACAAACATTCAGTTTGA
TCTTCTGTTTTTGAAAAAAAAAATGTTTGTTTGTTTTTTTCACTTGCTGATAGCAAGATTTCCCCCCTGGTCATTAGCT
TGGGGCAATTGGGTGAAACGACTTCTTGCTGCTTCTTTTCTCAATTCACCGAGTTTCAATCTGTACAGTTAAAATTTTA
CACCGTTTGGAAAGAGTTCAGCTTCTCGTGTGTGTGTGTGTGTGTGTGTGTGTGTGTGTGTGTGTGTGTGTGTGTGAGA
GAGAGAGAGAGAGAGAGAGAGAGAGAGAGCTGTTGTACACCGTTTGGAAAGAGTTCAGCTTCGTGTGTGTGTGTGTGTG
TGTGTGTGTGTGTGTGTGTGTGTTTCGTGTGTGTGTGTGTGTGTGTGTGTGTGTGTGTGTGTGTGTGTGCCTGTCAGTT
GTGTTCTGGTCATTTTGAATCTACTATGTCAGATTCCAAGTCTGGTTTAAGTTCTCTGGAGTGCATTGTTCTTGGCTTT
TAGAGTTAATCAACTCAGTTGGGTTTTGCCTGCAAGTTCCGACCCAGCTTCCGGTGTTTGCGTTTTGAAATGAGCTCCG
TTTTCAAAACTTTGTCGTTGCTAGTGAGGTGCTCTACCCTGGCATAGCCAGCTCCACCCTTCCTGTGATACCCAGATGT
TTTGGTGGATTTTAAGGTCATATCCATTCACTCACAGTTAGATGTAAACATGAGATTCTATAAGCAGTATTATTATTGT
ATTATATAATATATAATTATTAACATAATTATATAATGTATATTCCATAATATAATATATATGTATAATGTATAAAACA
TTCTATTACATCTTATTATATATTTATTAGTATATTATATTACTATATACTATATTACTATATACTATAATCTATAAGT
ATTGTGGGGCTCTGCACCCAGGCTCCTTATTCTTTGCACCCTTCCTCTGACATCCTGTTTCCCAGAGCCTCTCTTCTTG
GCTTTCTGGCTATGACTAAAGCCCCTCTTTTAGTTTCCTACTTCCCACAGCTGCACTCCTGGTTTAGGCCAAAATGCAG
AAAGACAAGAATGATTTGGAGTTTCTCATCCTCTGAGGACAGTTTTGAGCTCTCTAATAAAAGAATAAGAGTCCTGGGG
TACAGAGATGATTCGGTGGTTAAGAGCACTGGTTGGTCTTGCAGAGGAACTGGGCTTGACTTCCAGCACCCACATGGTC
GCTTGCAACTCTCTGTAACTCCATTTCCAGGGGATCTGACTCCTTCTGACCTCCTCAAGTACCAAGCGAACATATGCTG
AAAGTACATATGTGCAGGCAAAACAGTCATAAACAAAAAATAAAATAAACCTAATTAAAAACTAAGTAGATTGCTCCTC
TTCAGGGTCTTTTCCTAAGGACTGTCTGGTAGTCATACTTGCTTATTTTCCTTTTGAGTCGGTTTCTTTTGTTTCTTCT
TTTCACTAAAATTTCTGATATACTTTTTGGCTGTTACTTGTTATCTGATCTGTATGATGAAAATACTTGTCTCCTAGTC
TGTCAGGGTTTTTAATTCATTTATTTGTTTGTGGTTTTTCTTTTTTCTTTTTCTTTTTTCTTTTTTCTTTTTTTTTTTT
TTTTTTTTTGAGACACAGTCTATCTATGGACAGGGCCCTGACTGTCCGGAACTCACTATGTAGACCAGGCTAACTTCAA
TCTTACAAAGATCTGCCTGCCCCTGCCTCCCAAGTGCAAGGACTAAAGGCACACACCACCACAGTAGGCTAAAAATCCC
ACTGGTTTTCTCTCCTAATTTTTGTAGTGGGAATTAATGAGTTCTTTTCTTCATCCTTATTTTTTAAATGATTTCATGT
ATATGGGTGTTTTGTCTGCGTGTGTCTGAGCAACCTGTGAGTGCCTGGTAGCCATGGAAGGCAGCAGAAGATGTAGAAT
CACTGAATCTGGAGACACAGATGGTCGTGAGGCACCTTGTGGGTGCTGGGGATTGAACCAGGGTCCTCTAGAACAGCCC
GCAGTGCTCTTAGCCACTGAGCCATCTCTCCAGCATCCCTTTCCATCCCTCCCTTCCTTTGTCCCTCCCTCCTTCCTTC
CTTTCTCTCTTGACAAATGTCTCAGATGGCTGACACAGTGTCATAGATTGCACCCAGGGCCTTGTTTATGCTGGGCAAA
CCTCTGCCGACAGGACTTCATTTCTAGCCATTCTAACTATAAATAATCTAAATTTTCTCTGGGTCCCTTTATAGATTTT
TTTTTCTGGCTTTGTTTTATATTTAGTTCTCAGTTTCTCCCAGAGTTTTGATTGATGGAGGGATAATTGCATTGATGAA
GGTGAATTTATCCTACCCATTCTTCTATAATCAATTGTTCCCAAATGACTGGTCAAATATTACATAGCCTGCTTATTAG
CATCAGCCATGATGACAGATACCTATAATGCCAGCACTTGGGAGACTGAGGCAGAAGGATCCTGAATTTGAGGTCAGCC
TGGGCTACATAGTGAGTTCTAGGGCCAACACAGCAGCACCCTACCTCACAAAGCAAAATGAAAAACTCATCTGCCATAC
CACCTTTAATAAATTCACTTCTCTTTTATGTGTCAATTCAATTCTGTGTTTTATGTTCACTTTAACTAGGAACTTACTT
ATACCCAAATTAGCATTAAACACATTTATAGCTTGATTAATATGCTTTCTTTTATAAAATTCATTCTGCCCTTATGGAT
TTTTAAATTTTTACTTGATGAGCCCAGGATCTTATGTCAGGCTAAAATATTTTTTGAAAGAAAAAAAAAAAAAAAGCAT
GTGTACCAAATATGGACAGACCTTTTTTTTTTTTTTTGCCAGTATCCGTTATACAGTATAGTGACTACTTCTAGCATTT
GTATTAGGCATAATAAGTAATTTAGAGATGGCTTCAATAACATGAGAGGACTTGTAAGAATTATATGCAAACACTCATG
CATTCTGTAGAAAGGACTTTGAACTTCCAAGGCGTAGGAGAGAATTCTGGAGCCAAGCCTAAGCAACATTGCAAGGACA
TTTTAGAGGGTTAGCATGGTGACATATATCCATGCTATTCAATGCCACGGTGACTGTCTATTATAACAAATTACCTTTT
ATATACAAGTTATTGTTATCATTATATTTATTATTGTGTGTCTCGCACACATGTAATGTTATCTCATGTGCACAGTCAA
AGGACAGCTTGCAGGGGTAGCCTACCTCTTCCCACCGTGGGTTCTGGAGATCAGACTCAGAGCCCCAGGCTTATACAGC
AAGGGCCTTAACCCACCAAGCCATCTTGCCAGCCCATGTCCTTCAAAAAGGCACTCTACTTTACATAACAAGGAACGCC
AACAATTCTAAGTAGTATTTTATAATAATACTTTTAAAGGCCCAGAATTAGAAGTCTTACAGATTTTTGGAAAATGGAA
TAGTAATTTTTCCGTTTTATTTCCAAAATCATATCTGTGTCAGGCTGCCCTACCTCTTGTTCTGAGTGCGCGATTTATT
TTTACGGGGCTTTCCAATGTATGTGATTTTATTGCCAACAATAACAGATAACTTTGTTTGTATAGTCTGGACAGACACG
CCGCTTCTTCATTCCTGTTTGAAGACTGTCTAGGACAGTCCTCAAACATTGGCGGGGACAACTTCCATCCGCCCTCTTC
CCATCAGAGGGCTTCATTGGACCTTGTCTCTCTGTTTCTCTGTATTCTTTGTGCTCTATATATTTATTTATATTTGTGT
GTGTGTGTGTGTGTGTGTGTGTTTAAGCTAGTTCCATTCTTGAGTCGATTGGCCCAATTTAGGGGTTTTAGGGCCTCAC
AGAGCTTTCTTATCTCTTGTTCTTAGTGTTATCTGTCTGAAGAACTGCAGTGGTCACGTGACACTCTGTTCAGCGGTAC
TCAGACTGGCTTTTTTGCTCAGCAGTCCTTTTAAGCAGACCTTTAGAGGCAACCTCAGATAATCCATGACGTTGGGTGT
AAGGTCCGTCTTTTTGTTTTCCTTCTCAGGGAGGAGCTACACCTTGGTGGGGCGTCTGCCTGGATAACCTGTGCTGTGC
GGTGTAGAGGGAGGGGCGTGATCGAATCCACCCATCTCCGAAAGTGGCCCTCCCTCTTTCACTGGAATGCGGTTTTGGT
TTTCTGACAGCCAGTGTGCCCATAGCTTCTCCTTGTTCACTGGCGCTCTCCCAAAGGCATTCGGGCCCATGGCTTGTTA
CTAACTGTGGCTGTGGCTGAGGCCTCCTAGTGCGACATCTTGTTCGTATCGTTTCCCTCAGAGTGTTCAAATAGTTTCA
AAATAATTTTAAACACGATTGTGGTGGAGTTTTGTCTTTTTTTTTTTTTGTAACCTAAAAATAACAAGAAACAGAAGAT
TATTTTTTCCATATGCTTGTGCCTTGCTATAATTTTATTACTAATTTGTAATTATGCTTAACTACCATTCATGAATATG
GGTGGGGCTTAGGAGATTTGTTTTGTAGTCTATTATAGGATTTTCTTATTAGTTTACTATAGGATTTGCTATTCACGCC
TCTCTTCATCATTTCCTTCTAGATCACCCAGGGTGTACGGGTACAAGTGTACAAATCGACATTTATCAATAATAGATCA
TAAGGAAGATCATTTTAAAACAATTCCTTGACACTTATTAAAGACAAAATTATACTAATGGGATGTGTGTATAGAGATC
TAATAAACCGGTTAGATACACACTAAGAACTGTCCGTGGAAAAAAATTAGTCTTTTTTTTTTTTAATTACATAATTAAA
CCGATGTGTTTCTAAAAGAGCAGAAAAGCACAGTAGCGTCAAACTTGAGCCAAGGCATCTCAGTGAGACCTTGTCGTCG
TGTGTCGAGTGTACAGTGCAAGGCGCAAAGCTTCCGGGAGGTTGGAGGTTGCCCTGTGCAGCATGGGTGAGCATGATCG
GAAGGGGTGAGCGCGATCAGAAGGGGTGCGCATGATCAGAAGCAGCTTGGGTTTCATGTCATGTTTGTCTTGGGGTTCA
CGTTTAGTCCGTGCGCATTCAGAAACCTTTAACCTCAGCCACATTTTCACAACCCCCACAGTGAGTTGTGTGACCTCAG
GTGGGGTCATGACTTATTTTAAGAAGCTTTGCTATAATGCTAAGTCTTGTGATGACGCCAGCTGGTTGTCGTTGTTGTT
TGTTTATTAGCGTGTTCATTTTCTTTTGAGTAAAATGTCCCAACTTTATCTTGGTTTCCCTCAACTTTGCCTTTTAATA
TTCTGAACCGTGACCTCTTATGGTAAAACTGCCTGAGCCCTAGTTCCTTAGAGACATTTGGGGGAAGCTGAGGAGCGCC
CTTGAAGTCCTGTTTCTGTACTAACAGTAACTTTCTGTATTAACAGTTACACAGTTGTGATGCACACCTGCGATCTTCA
TTTGAGTAGGAAACATGTTCAGGTGTAAAGGCTTATAATCGCCATGTGCTTTTGTTACTGAGTGTAATCTTTATTACAC
ACCTTATTTAACATCTTATTAGGCTTGTTATTAGACCCCATTCCATGTCACTATGTGGCCTGTAACTTTCTTTTTTTTT
TTTTAAAGCCTTTTTTTTTATAGGATATTTTCTTTATTTACACTTAAAATGTTATCCCCTTTCCTGATTTTCCCCCTGG
AAATCCCCTATCCCATCCCCTTGCCCCTGCTTCTATGAGGGTGCTCCCCACCACCACCCTCTCCCACCTTCCCACCTTC
AGTTCTCCTACACTGGGGCAGCAAGCCTTCACGGGACTAAGGGCCTCTTCTCTCCCATTGATGCCCGACAAGGCCATCC
TCTGCTGCATATGCGGCTGGAACCATGGGTCTCTCCATGTGTACTTCTTGGTTGGTTGATGGCTTAGTCCCTGGGAGCT
CTGGGGGGTCTTGTTGGTGGATATTGCTGTTCTTCCTATGGGTTGCAAACCCCTTCATCCCCTTCAGTTCCTCTCCAAC
TCCTCCATCAGGGCCCCTGTGCTCAGTCCAATTGTTGGCTGTGAACATCCACCTCTGTATTTGTCAGACTCTGGCAAAG
CCTTCCAGGGGACAGCTATATCAGGCTCCTGTCAGCAAGCACTTCCCAGTATCCACAATAGTGTCTGGGTTTCTTGACT
GTATACAGGATGGATCCCCAGGTGGGGTTGTCTCTGGATGGCCTTTCCATCAGTCTCTGTTCCACACTTTGTCTCTGTA
TTGCTCCTGTGAGTATTTTGTTCCCCCTTCTAAGAAGGACCGAAAAACTCTTTCTTTAATTCATGGACCTGTTTTTTTC
TATGTATTCCTAACTTTCCATTCAGTATTTCACCATCTCACACACATCTAAAAAATGTTCATTATTTTTAATTATGTGC
ATGCATATATGCTAGTGTGTGTGTGTGTGTGTGTGTGTGTGTGTGTGTGTGAATGCAGGTGCTTGCCAAAGCCATAGGT
CTCACAAACACACACACATGCACATATGTATACACATATGCACACACATACCCTCATGCATGTATGCCAGTGTGTGGAT
GTGTGAGTGTGAATGCAAGTGCTTGCCAAAGCCAGAGGTCTCACACACACACATGTCAAAACACAGTCCAAAAATGGAA
CCGTGTGAGAATTCTGAGTGCTTGTGAGAACACTCCAAGAAAACAAGAATCTTGTGGTCTCAGTTTCTTCAGAACACGG
AACTGTCCTTGGAATGTGTTTCCGTGCTCCTCCCAGGTGTGGTGGACAGGAGTGAGGTTACTGCAGACTACTCATTACT
GCGTGCCGCTGACGTGCCGCTGGCGTTCAACTAAATGTCTGAACTACCCTCTCTAGGTTTCTGTGGGAAAACATGTTTT
TGCCACATGTGGCTTGTCCTTGTGGTTGCATACAGCTCGACCTCGTGAGAACTTTACTCAGGTGACCCTTCTCAACCCT
CAGAGTATACACTGTCTGGGAATCTGAGAAAAAAATGGTTATTGCATGAGACTTAGTCTGCCTGATTTATTGGCTCCAC
TCTCCCAGGTCCTACCACCTCTAGAGTTACGTGTATACACACACACACTATATTTTGATCTTATTCACTAGTTTTGTTC
TTGTTATCATTGAGTGCTGAATCTTTTTCTATCCTTTTAATTTAAAACTTGTACTTTAATATAGTATTCTTGAAGGAAA
AAAAAACAGCATAATTTTCACCTTTTCAAATACAATCTGATAGTCTTTTTAAAATAGCCAATATCAATGACATCCAGTT
ATTGATAATTTCATGAGCCAAAGAACACAGTTGGGTGAAATTTGGTTTGTAGTTACTGTGATGAGGGCAGAGGCAGCAC
CCTAAAGTTCCTCATGGGGTGTGGTCTTCCGTGTTGTGACAAAGACCTGTTCAACTAGAGCTGTATCCGTGGTACACTT
TGACGTGTGAGCGCCAACACAGTAAAAAGCACTGGCTCCCAGCTTCTCTTTCGAGCTGCTCTGTCTCAATAATAACTCC
TCTTTGTGTGCCAGCTAGCGATGTCTCACTGTGTGCCAACTTGTGATGTCTCACTGTGTGCCAACTTGTGATGTCTCAC
TGTGTGCCAACTTGTGATGTCTCACTGTGTGCCAGCTTATGATGGCTCACTGTGTGCTAGCTTGTGATGGCTCACTGTG
TGCTAGCTTGTGATGGTTTAGTGTGTGCTAGCCTGTGATGGCTTACTGTGTGCTAGCTTGTGATGGCTCACTGTGTGCT
AGCTTATGATGGCTCACTGTGTGCTAGCTTGCATCTCTACTTTCATCTCTTTGTCTCTTGCTCCCACAACTCTCAGTCT
CCATGTCTCCCTAACTCTTTCACATTCAGTCGAGCCAGATATACAGGTCAGAACAGTGACTATTACACAGTGAGCCTGG
AAATGGGAGCAGGGACAAAGATGGAGGCTAGAAATTGGGCTGAGCATAACTCTCAGAGATTATCCTAACATGGCAGTGA
AGAAGAATAACAACAAACAGCCTCCCACCTCCACTATTCTTCATACTCCTGTGGCAGCTGGAGCCTAGTCATACTCACT
GCGGGGATTTGATTTGTGTGTCTAATGAGGACCAAGACAATCCAGTTCTTCCTTTCTGTTCTTTTTCCACTTCCTTTTG
TAGCCTGCTGCCTTTCAAGCTGCAGACCTAGTGCCTTGTGGAGGCGAGGGAGAGAGGACACTGAGGCCTTGGACCAGAA
AGCAATGCGAATATGGGGAGACTCTGTTAGACTCTCCTATGCATGGTCCCATTGTGTGAAATGCCTTCCATCTCCACCG
CACAGCCATGACACATACATCTATTCTTGTTTACTTATTATTGTGGTGAGAAGGGCAGATAGTTGATCACCTACAGGAT
GATACAGGGCCAGCCTAAGAGGCAGATGATGCTAGAAGTTGTGAACCACTGTGGGGCCCTGGAGGATTGGCAGCAGTAG
GCCCAGCCTCTGGCACTCCCCGGAGGACCAGGGAAGATACTGGCCAGGCCTTAAAAGGTGAAGGAACATGGCTGGAGGA
CAGAGGCAGAGAAAGAAGGTGTTCTATGCAGACTGCACGGAGTGGAGCAGACCTGGATTAGGGCAATGTTGAGAGGCAA
GCAAGAGGGACTAGGACTGTGGGTTGGTGCACAGCACCTGGGCTGTGGTCTGGCTTTATGCCCCTGGCCACCGTGCCTT
CCCTGCATTATTGGGTCTAGAGTCTCTGTCATCAACAGGTGAGCAGACTCTCAGAGTGCCTTTGGCTATGTCAGCAGCA
GCTGCTCCTCTTTTCTGTGCTCCTTCCCCTGGTCCCAGCCCTCTAACCTAGTTATCAGTCTAGGCCCCTCTCTAGATAC
TTTAGTTTGTTAATAAGTAAGTTCCCTTGGAACATAGAAGCCAGGGGTGGGATGTAGCTCAGTGGTAGAGCACTTACCT
AGTAATACAAGGACCCTGGGTTTGGTCCCCAGCACTGGAAATAAAACAAACAAAAATAAATAGAACCAAACTAAAAATC
TAAAAGTGATCTTTTAAAAGAGAGAGAGGGAGGTATTTAAGCCATGTCTGTCCGTCTGCCTGTCTTATCTTGTGCTGGG
GATCTAATCCAGAGCCACATCAGTGTCAGGCAGGCACTTTACCATGGAACTTAGTCTGTTTGCTCATTTGTGTCTGTTG
CTAACAACTTAAAGTCTTGATTACTAGACAAGCACCTGCCTGTGCTGACTAATTCTTATGAGACCTTGACACAAGCTAG
GATCATTGGGAAAGAAGGGAATCTTAACTGAGAAAATGCTTTTATAAGATTGGCCTGTGGGCAAGCCTATGGATAATTT
TCTTGAGGTTGGTGTTAGGAGGGCCCAACCCATTATAGACAATCCCACCCTTGGGTTGGTGGCTCTGAGGTTTATAAAA
ATGCAGGATGGGCCAGCCATGGGGAGCAAGCCAGTGACAGCTCTCCTCCAAGGCCTCTGCTTCAGTTCCTGCCTTCAGG
GTCCTGCCTGAATTTTCTGGATGACGGACAACAAATCCTAAGATGAAATAAACCCTTTTCCTCTCTGAGTTGCTTCTGG
TCATCGTATTTTAACACTGCAATAGTAACCCTAGCTAAGACACTGATGGTAGCCATTGAGAGAAGGAGACTAGAACGAG
ACTTGCCACATCAATCTACTGCCTGGGTGTGGAGAAGCGTCACAAGACCTCTCTAAGCAGGAAAGAAACACCCAGGTTT
GCAAGAGGATATGTAGGAAACAGTGACACTTAAGTCAATTTGGAAGGCTAACTAATGAGTCTATATTCTAACCATCCCA
CTGGTAAGTCTCCCTTTCCGATGACTCCATTATTGTGTAGGGAAAGCAGAGAGTAGAAGCATGCAGGTAGAAATCCCTT
TGGGGGGTGTGAGTCGAATGACTCACAGGGGTCGCATATCAAATATCCTACATATCAGATATTTACACTAAGATTCATA
ACAATAGCAAAATTACAGTTATGAAGTAGCAGCGAGAATAATTCGACAGTTGGGGTCGCCATAACATGAGGAACTATTT
TAAAGGGTCGTGGCACAGGAAGGTTGAGAGTCACTACCCTATAGGGTTCCCAGCTGCCTGCTTAGGTGTCCCTTTGGTT
TGTTCCTCAGTGTGTGCTCTGCAGCCTGGTGCATCAGAATCTGGGTGCTGCCTTTTAAATATGAACTTTAACTAGATCC
TCAGCTGGTGAGTCTGCTCATTAAAATCCTGATGTATGGTTTTGGAGAATGTGGCGGGAGTTGCTACATAGTCCACAGG
TTTTGTTTGTCTGTCTTTCTTTCTTTCTTTCTTCCTTTCTTTCTTTCTTTCTTTCCTTCTTTCAAGCTCATGCCATCTT
CATATTAATCTTTTCTCGCCCTGCTAAGACAGAGCTAGGCAGTCTTCCCTGAGTGTGCAGAGGGGTGAGATGTCCCTTT
AGAAAGGGGCAACCAATGGCCCATGCCTTTAATCTCAGCACTCAAGGGGCAGAGGCAGGCTGACCTCTAAGTTTGAGGC
CAATCTGGTCTACATCGTAAGTTCTCGGACAGCCAGGATTACATTTAAAATAAAAGAGCCAAGATCTTAGGAGCTAAAA
GAATGAAGCCTGCCCAGTGCTCACATACTCCCAAGGTGGGGCATCACCCCACCAGTCAGTCAGTTGTCCCTAATGGGGA
TCCTTCAGAGACACCCCCTCCACACAGGATCTTGTCTTCTTATACATCATCTCCCCTCTCTCTGTAGCCAGGACGTGTG
TGCCACAGAAGAAGCAGAAGACCGAGACAACTCCAGAGTCACTGGTACACTCCTGTCCCCTGTGATCACAGCAGGAACA
CTGTGGAGAGTCCCTCTTCCCCTTAGGGATACCCAAAGCACAAAGCTATCTGCCTGATGAAAGCTGGAGGGATGGCTCA
GTGGTTAGAAGCACTGGCTGCCCTTCCAGAGGGCCCAGCACCCACATGGTGGCTCCCAATTATCTGTAACTCCAGGCAC
AAGGAATCTAATGTTCTCTTCTGGCCTGTGAGGGCACAAGGCACACATGTACGTAGACGTGCATACTGGCAAGGCATTC
ACATACGTAAAAATGTTTTGATAAAAAAGAAAATGCTTTCTCCCACTACAGCTGTTAAAACCAGACCAGAGATGCCAGG
ACCCTTAGAGCTGGGGCCCCTACAGTGTATCCATCCTCAAGCGGTTCTGGTAAGTGCCTGGAGTGTTTTACTACCACCT
AAGCAGCAAAAGTTTCTAGAAAGATCCAAGCCTGGCAACAAGGGACATGTTGGGACCCTTCTTTAAGTTCAGTGCCCAT
GCATCTAACACAATGACCCTGGTGGCACTCTGACCTGTTTTTAGAAAACCATAAGGAAGAATTCATCTCTGAAGGGGTT
TCCTTTGGAATCACCAGGCACATACTTGGGGTATAGAAATGTATCTCATATAGAAATGTCTCTTATGTGCAAGTGTAGA
TGAGGGGGCAGAAAGGCTTGGGAAACTCACCCCCAGGTCAACCCCTTCCCACCTGGGGTATTCAGCAGCCTCCTGTTGT
TTCCAGAGTTGATAGGGTCAGAGCCTTGGCCCTGTCAGCCCTAACAAAGTAGGCGAGGTTACCTGACCTCAGTCAGCCA
ATTAAATAGACAAATAAACAAGGACAAGCAGAGAAAGGTGATTGATTTCATGTGGCCACACAGGGAAGAGGAAAACCCC
CAGTCCATCTTTGGGGTCCCAACATGAACCTGGGGTTTAAACAAAGGCAAGAGGTCTTTGTAACTAAGCAATCCCAGGA
AGGGTGTGGTCCGTCCCATCCCTGAGCGGTCATTGGCTCCAGTCTCTCATGCAAGGTGGTCGGCAAGTTTCCCAAACTA
TGTGAAATCTCTTCCCTGGCTGGCTGGCACTCATGCTTTGACTTTTCCTTCCTCCAGATGAGACTCCTGGAGAAATGCC
CATTTCCTGGGACCCATTATCTACATAGTCTGGAAAGGGCAGTGATTGCCTCTTTAGAGCTCTCCATGCCTGTCAGGAC
CATCTGGATGCATTTCTGAGGCCAGCTGTGTCTCCAGCCAGTTCAGTACAGAGACAGATGCAGCTCAGCTCCGCCAGGA
CCCGGTGTGATACAGAGCTGAAGAGGGAGGAACAGGCCTGATGGGTCCCAGGTGCATTCTGTTGTCCCTTTATTCTTGT
TTCTGTTTTTTCCTCCCTCTACCACCCTCGGGACTACCTCCTCTCTACCCTTTCGTCAAGCTTGGGGTATTAGTTACTT
TTATCACTGTGACAAGAATGCCTTACAAAATCAGCTTCACGGAGGGTTGATTTGGGTTCAGTTTGGAGGTGCAGTCCGT
CATAGTAGGGAAGGTACGGTGGCCATAGCAGGCACAGCAGAGGTGTGGTGGGGGCATAAAGTGGCTGGCTGGTCACATT
GTGTCTAGAGCCAGGAAGCAGAGAAAGGAATATGGTACCCTGCTGCCTTCCTCTTCTGCCCCTTTTATTAAGCCTGGCA
TCTTAGCCCATTGGGTGGTGCCACTCACATTTATAGCCCATAGGGTGGTACCTCCTCAGTCAGGCCTTACTGGAAACCT
CACAGACACCCTCAGAAGTGTGTCTCCTAGGTGATCCCAAATCCCCACACACTGACAACAGAGGCTAACATCACACTTA
GTGAGAAATGAAGTGGATTTACATCTTGTCCCAGGAAAAGAAGTGAATTAAAAGACACTGCTCATCTACACAAAGCCTT
CTTGTCCCCCAGGGTAAATGTAAGGAGGAGGAGGAAGAGGAGGAGGAGGAGGAGGAGGAGGAGGAGGAAGAGGAGGAGG
AGGGGAGGAGGAGGAGGAGGAGGAGGAACAGGAACAGGAACAGGAACAGGAGGAGCAGGAACAGGAACAGGAAGAGGAG
GAGGAAGGGGAGGAGGAGCTAGAGGAGGAGGAGCAGGAAGAAGAGGAGGAACAGGAAGAGGAGGAGCTAGAAGAGGAGG
AGGGGCAGGAAGAGGAGGAGGAACGGGGAGTGGGGAGCACTCTTGGACAGGGGACTGTGGTGTTCCCGTGCTGTACAGA
TAGCTGTTGGCTAATTCACTGCAAGTCCATTAGGTTAGAGCCTGTGTTCATTGTATGAGAGCCTCTGGACCCAGCATGG
CACCCAATACACGACACATATTCAGCCAGCGTTACAATGACTTCATAACCAAAGACCTTATGTCACAGCAAAAAAACGT
AAGGGGCACTAATGGAAGTACTTCTGCCTCCCAGCCCAGCTGAGGGGGCAGGGGCCCCTCAACCTTCTCTTCATTGAAA
CCAAAGGCCCTGTTCTAGGACCTCTGTCATGAGTGACTGCCCTATGAGGCGAACAAATACCACCTTGGGAAATTCAGCT
GGGCCCCCTCCCATCCCCCACCCCCAAAAGATCATCCCAATTGGGACAATTTACACAATAGAGGTCCCCTACCACCCAG
CAACTCTGCGTGTGGCCTTAGGGAGAAGCTAGAGCATATGGTCTGGGGAAGGTTAAGAAACCCTTATCCTTGTTGGGTA
AGGCTTTCCAGATGTGCACAGTTGTGGGAGGAACACTGCATCTCTGAAAGTAACCCCTCAGTCATTTGTCCTTTAGAGA
GATGTCTGGTGGAACCCTGCCTAGGCTTGCCATTGAGACCTTGGGAGGAATGGTAGACATTATGTTATTTTACCCCAGG
GAAGGGATTTTAGCAGGAACCTCTCATCACTTAGCCTCAGGCGGTTGCTTCCTTCCACATACTGTGTTCTGTTGCACAC
GAGTTCTGCCTGCTTTCTCAAGACAAAGACTGGCGTTCCGATCCATCAACATAGACCAGAAGCCCTTCAGTGTCTAGAC
CGTTAGTTGACTCATCCCCCGCCCCAGCCCCCATTCTATATTCATTTACTCAGCATGCACCTGGTGTCCCAGGTAGAGA
GACAGACAAGGTCTGCAAGGGCCTCAAGGTCTGGGCTCTGCGTGGTGCAGTAGTCAGTTCTTTCCCCAGCCTCTGAAGG
GGTCCGCCCTCAAAAGTATTGAATAGTCACAAGGAAGAACATGATGACTGATTTTCTGTTTCAACTTAACTAGGCTAAG
GGATGTCCAGAGAGCTTGTTAAACGTTACTCCTAGGTGTGTCTGTGAGGCTGTTTCTAGAAGAAACATTTGCGTCAGAG
ACTATATATATATATATATAAAATCCCCCTGGCCAATATGAGTTCCAATACACTGAGATTCAGAATAGGACCAAAAGGC
CCACGAAGCTGTCCTCTCTCGTCTTCAGATGCCAGCACCCTGCTTCTTAAGTCTTGGATTTAGACTTTAAGATGCTAAA
GGCTCTCAGGTTTTAAAACTCAAAAGTATATCAGCATTCCTTGCTCTCTGGGTTGCAGAGGTCAACCATGAGACATCTC
AGCGTTGGTGACTGTGTGAGACAATCTTTCCTAATAAGTCTCTTTCCTCATGTCTAAGTCTAGCCTGTTGCTTCTGCCT
TTCTGAAGAATCCTAAGTACGGCGGGGATAGGTAGGCAGATGCGAAGAAGGACACACAGACAGCGCTGTAAGACCAACA
CCAAGCAGCCGTCTTTCAGAAGTTCTGTGCAGGATGAGTGTGCATTCCCCCGTGTTCTCTGTCTCTGGATGAAGCCTTG
AGACTAGCTCTGGCTAATGTAGTATCACTTCCTGGCCCAGTGCCTAGGCTAACCCCACAAACTCTTTTGCTGCTATTGT
TGTTGACTGAGACCGGATCTTTCTATTATGTAGCTCTAGCTGTCCAGGAACTCAGTATGTAAACGAGGCTGGCCTCTAA
CTCACAGAGCTCCGCCTGCATCTGCTGGGACTAAAGGTGTGTGTCACCATGATTGTCTGTGTCATGAATTCTTCAACTG
CTTTCAGTCTTTTATCTCCCTGAATACCGACCAATGACCTAGCCGAGATGACCAAGAAGTCACAAGCTAGGGGTGACCA
TTCAACCAGGGCAGGGAGGGGGGAACCTCACAAAACTATATAGTTCCTTGACCTGTAAAAAGGTGATTAAAGGCAGTGA
GGGCTTATTGGGATAAAATCCCAAAATATAAGATGCTGGCTTAGTAGTGGGTTGTAACAGTGAGGGAGCTGGTACTGGA
GTCTGGAGAAGGGACCCCTGTTGTCAGAGGCAATTGTTTGGTAGAATTAGTACTTTGTCAAGAAGAGAGAGAGTAAGGG
CACACACTACCAGGCCCTCAGGAACAGGAAGGAGTAGGGTGAGCAGCGTGCTGGATAGTGGGCACAGATGTTATTGTGT
GGGTTGACAAGGCATGGCAAGAGTGTTGCTCGCTAAGGAGAGAACCAGCCATTATCAAAGTAGAAGTGCAATAAAATTG
AGATATTCTAAAATAAGAGTGGAGTGGAGAGATGGGAAGAGTATTTTGAACCTCTAAGAAGGAATAAAGTGAGCTCTGC
ATACCAATGGCTCAATAACACATATTACTTAATGAACATAAATACATTAAAGTCTTTTTGTTTGTTTGTTAGTTTTGTT
TGCAGTAGAGCTTAGAACCTTGGCTCTTACTGTGGTACAAACACGAAATAAGAACAGTTTTTGGACATTGGACTTCACT
GTAATAAGGCTGTGCTTGAATGTCCCTCACATCACCAAAGGATTTTTACCTCCATAGTAGCTAAGCTAAGAGTTGACAG
TTCTGCTGCACCAAGGAATGAATTGTAGGCTCAGTTTTTGGACACAGATTTCCTGCTATGGTCAAAGCTATTTGACTTG
AGTGATTGTCGTCATTCTCAGCCTGCGGGGAACAGGTTACATTCGTTTAAGAAATGGTGTGTGTATTAAGATGCTCTAT
CCATGAAGTCATTCATCAACTGTTTCAATGAACAATGGCTCTGCTTGGAGAGGGTAAGATTCTGGTTCATTGGCTGTGA
TGACTCTGAAAAGCACATGGCAAACATTTTAATTTGGCTTTTTTTTGGGTTTTTTTTTTTGGTTTGTTTTTTTTTTTTT
TTTTTTTGGTCAAAATGGGTAGGCCCTGAGTAGCTGTGGTTCTGCTGAGAGACAGCAAGTGGTGGACGTGAGAAAAGAA
AGACAACGTTAAGTTTCGAGAGCATGAGAAGAAAGAGAGTGTTGTGATTAATACCCGGAACCAACTCAGAGAAATTAGC
TGTTGAGTAAGTGCCTGAAGGAATTACAGTTCATAATCTACCCAAGCCACGACCACAGTGCAACAGAAATGGCTCTGTC
TGTTCAAGACAAAACAGACATCAGCGTGATGGTGGTGGAAGAACGGGAAACGAGCCTGGGCCACTCATTTCTCCAAGCA
CAGAACGTTTTGAGATCTCCTCTTATCTCTCAGTTGCCAAGGTGTTCACAGCAAGTTCCAAGAAAACAGGCCATTCCCA
GGGGACTGAACGAGTTGCCTCCCCACAGCTTTTGACTCATAAGGTCTCAGGACATACATCACCACATGGATGACTAACA
GAGTCTTCAGGCTTATCCCATCAAAAGATGGATATCCGAGACTCCCAATGCTGAATGAGATGGATTAACAAACGGACCA
AGTTTACTTCCACCCAAGACAGACAAAACTTCTTGACAAATATGTAACAAATCTCTCTTAAAGCGTTAGACAACAAGCA
ATTAGGAGCTGTAATCTCTAACACAAGAGAAACAGCTGGCTGAGGTGCATCTCATCATGACAAGCTCATTCCAGAAGGA
CAAGCTAGGGAGGACTAAGAGAATCTCCCTAAGCTGAGAAGATAGGACTGGAGCCTACGAAGGCTTGATTTTGCAGAGG
ACATCATTAGCAAAGAGAAGCTGTAGGAAGAAAACTCTAGGAGCACACAGAGGGTCCCTCAGGTTTGTTGAGCCACACA
AGGCAAGTAGAGATTACCCCACCCCACCCCACCCTCAAAAAAGACCAAATTGGGGAAATCTTTGTAGCCCAAGGAAAGA
AAACATTCTAGCTGTGTGGACCAGAATGGAAACTTTTATAGTCAGGTAGACTGCGTGGTGCCTGCTTCAGTGACCAGAA
AAAAATTAGCCCCAGACTAAATGCTACTCTGATTTTAATATAATCACATTTAAAAGCAAGTCTTAAAAGAATTAATCAT
GGAGTTGGACAGGTGGCTCCGAACTTTAGGGCATATACTACTCTCGCAGAGGACCCAAGTTTACTTTCAATCCTCCTAT
CAGGGGCCTTGGCCCTCCCACCCCATAACTCTGGCTCCCGGGGATCAAATGCTATTTTCTGGCATTTTCTGGCCTCTCT
GGGCACCTGTACTATTGTACATAAACCCACACAGAAGCACATAGAAGTATATACATAATTAAAAATATTTCTTAAAAAA
AAAAAGAAACAGATTTACTCACATCTCATAACAAAGTTCAAGCATGTTTATAGGAAGACCAAAACATGTAGTCCCAGGC
AAGGTACTATTTACATGCTGGCATACACTCAAAGATGATCAGATTTGAAGAGATGTGGAGAGATATGATACTCCATGCT
GAGAAGCGGTTACCAGTTAGAACTAACCCAGAGCGTGCATAGATGATAGGATCAGTGGGCGGAGACCCTCACACATCCC
TTCACTACACCTCGGGGAAGACAGATGGAGGAGCAACTGAATATACATACAAAAGAGCTCTAAGATCCAGACCGAATGT
ACAGGCATGCAAATGCCCACAGCTGAGGCTTATTTGATATTAGATATGGTGGATTGGGTATGAGAGGGAGACGATAAGG
AAACTGAAGACACCTGGAAGCACAAAGGAGACACACATACACACCAAAACAAAAACACCCCCCCCCCAAAAAAAAAACC
CCACAGTGAGCTCCGGGGTAACAGTGAACACCCCAAGGCATATGTCACTGGAGACCCCACAGAAGTGACTCTGTAAACT
ACTGACTGTGGAAACCACATTAAGCACTTTCTTGATGCTCCAAAAATGACGTAAGACAAGAAGTCAGAACCGTAGCCCA
AGGGAAGAAAGATAGCGGGTGGCTTTCTTGTTGGAATCTACGAGAGCCAGAACAACATTCTTTTGCACAGAGATAAGAG
TGTTTCTTACCTACTAACAATGTCTTTCAGTAATGGAGAAATAACCTTTTCAGGTATTAAAAAAAAAAAATCCCAAAAG
AAGTCGTTGCCAAGTGCGAACTTGCACTGCAAGAAAGATCAAAGAAGTCCTTGGGACAGGAAGGAGTGTGCATTTGCAC
AGAGGAATGAAGAACCCTCCCAGGAACGGCCATGTCCTGAACATAAAAGGGAGCTCTTGGGCTCACTGAGCCCTTTAAG
AGGAACTTGTGTCATTAAAAGAGAAAAGTATTAGCATTTCTTTTTTTTTGGGGGGGGGGGGTAGAAACACACAGGGTTT
CTCTGTGTAGCCTTGGCTGTCATGGAACTCACTTTATAGACCAGGCTGGCTTCGAACTCAGAAACCCGCCTGCCTTTGC
CTCCCGAGTGCTAGGATTAAAGGAGTGCACCACCATGGCCCGGCAGTGTTAGCATTTCTAATCAACTGTGAACAATGCT
GGGCTTCCTTTTCTGCCCTGTACAAGGTTACTTTGAGGCTGTGCATCCCTGGCTCTCCTCTCTCTCCTCCTTCCCTCCC
GCCCTCCACTTCCTCTCCCTCAGCAGTTGGCCTTCTAGGATACAAATAGCCAATGGATACCTTAAAAGGTGCTCAATAT
CCTCAGCAACATTTTCTTTTCTTTTCTTTTTAGAATAACATTTTAAATTTACTTTAGGAAATTTTTATACATGTAAACT
TGGGTCATATCCAACCCCCTACTTTCCCCTTCCAGCTTTCCCAAGGATCCACCAACCTGTCTGCCTTCAAATTTCATGT
CTTTTTTTTTCTAATATCCTTCCTTCCTTCCTTCCTTTCTTTCTTTCTTTCTTTCTTTCTTTCTTTCTTTCTTGCTTGC
TTGCTTGCTTGCTTGCTTGTCTTTTGTTTTTTTATGAATAGCCCTTTGCATTGAGTTAGTGCTGACCATATGCACATGA
AGGCTGGGCCGTCATTCACTGGGGCATGGACAGCCCTCCAGTGACTTCATTCTCAAAGGAAAGTAACTCTCCCTCCCTA
AGCAGCTAAAAAATGCTAATAGCAGCTCAGCTGGGGGTGTGGCCTGAGCCCTCCCTCACCCTTGATGGACTCCTGAGTG
GTTCATGCAGGACACGTTCAGGCAACTGTCCACATCAGCCCTTCCTGGCTTTTCTTCCTCCTCTCTGGATCTTACTTTT
TTTCCCCCACCCTTTGATATTTCCTGAGACTTGGATTGGTCAGGGGGGTTGACATGAATGACCTGGTTATGACCGAGTA
GTCCCAGTCACTAACTCTCAGCACTTTAGCCAGTTATGAATCTCTGCGTTAATCACCGTCCGCTGCAGAAGCGTCTCTG
ACCGAGGCTGAAAGCATCACAAACTTACGGGCATAAACATCAATATTAAGAAGATAGTTTGACAAAATGACCACTTTCC
AAAACACCACCAGTGGATTTTCCCTCCCTAGGGCCTGTGACCTCCACACTCACAGGCTTTCGACGGGGTTTCTAGTACC
ACACATGAATTCACGTCTGTGGAGAAGGTCTCAAGTCCTATCACAAAAGGGTTGGTTACCCCAATAGCCTTCATACCAC
TATTGAAGCAGTGGGCACATCTTGCCTCATAATCTGATGTCAGTGCAAATTTATATGATTTTAGGAGTCTGTGTCATCC
CAATAAGAACAGCTACCATCAAGAAGACAAACAGCAATACTTGAAGATGACGACGTGTGCAAAGAGAAACTTCTCACTG
CTGGTGGGAGTGTAAGCTTGTGCATCCATCATGGAAATAGTGTGTGTAGATTTCCATAAACAAACAAACAAACAAATGA
TAGGTAGATAGATGCATACATACATAGGTAGATAGACAGACAGATAGATAGATAGACAGACAGATCCTTTGTATAATAT
TATACCATTTCTAAGCACATATCCAAAAGACGTCTTATTCTACCAGAGAGGTGCTCACTTCTCTATTCGCAATGGCAAG
GAGATGGAGATAGCTTAGATGTCCATCAAGAGATGAGGGGTTAACGAACATGTGGTATATATATGCAATGGAATTTTAT
TAAGTCATAAAGAAAAATGAAATTATGAAAATTTCATGGAAAATGGGTGGATCTGGAAGTTATTATATTAACTGGGGTG
AAGACAGAAAGACAAATACCACATGTTCTCTCTCAGAAACGGATCCCAACTTCTAATTGTTATGCCGTGTATTTGTGTG
GGACTGAGTGTAGATACGGGCCATAATCTGAGAAGGGACCCTGAGAGAGACAAGGAGAGTTGAAGGGAGAAAGACAGGG
AGGGTAAGAGAGCATTTGTGATGTGGACGTGGAAAGGGAACTATTGGGCACGGAAAGGTACCACGGTGGGCCATGAGGT
GGGGGAGGGGAGCGGGGAAGGAGGTTCAGCCAATACTGTGTATGAAAATGACATAAGGAAACCTGTTACTTTGTACGAT
GATTTAAAATCTCCCTTCTCCTTTCCTGCTAAAAGAGAATTGTGTGTGTTGACATGGAAAATATTTATGTGTGCATCTG
CCCGCAATTATCCCGTCTTGCAGAGAGAAACAGTGAAGCCACTTTGGTTGTCCTGTGCAGAGAGTATGTTCTGCCCTTC
CCAGCCGGCATCTCAACATGCTTGACCAACTGACCACAGCAAGGAATTTCCCATTACACACTTAGTCACATGGATGCTT
TGCGGCACTTAGTAGTTTAAGTCCACCAAATGATGTACTGGCTTAGGAGCAATGTGAGAAAGCCTACAAGTGTAGATCC
TCTGAGTGATCGCTCAGACTCTCGAAGATTATCTTCTTCTGTGAGCCCCAAGTAATAACAATTTCCGGCCCTCTGCTCA
GTGTTCAGTGTGTCTGAGAGCAGAGACAACCAACCTCACTCCATCTTTTGCGTAACCCCTATGTGACACTTAGGAAAAG
CAGCTAGGTGGCCCCGTGACATCCCCAGCTATGGTTTCTTCTGCCTCCCAGGAATGTGGTCAGCTGGAGGCATTCCTCA
GGGTCACCAGTCCCTGCCCTTATGAAGCAACAAGCCATTCCTTGGTGTTTACAGAGCAAAAGCCACCATTTGAGAACAA
CTGTGATGTAGAGACAACAGTGAAAGCAAAGAGTAAGTCCCATTGTTACCTTGCTACATCTGAAGAAGGGACATCATTC
CTGGAGGAAATGAGCAAAATCAGGACCCTTTAGCCCTGACTGCTAGGCTCCCCAGGTGCTCCAAGCAGCAGCACCCACC
TCCCATCCCCCACCACTACCCGGTGCGCTCAAGCCAAGCCCTGCCTGCCCATGGGCAGCTACAGGAAGAGACTAACAGC
ATCCTTCTTCAGGCACTGCTGGCCACAGAATGCCAGGAAGAGGGAAGTGGGAGGCAGAATCCAGAAAGAGAAGGAGTCA
GCTGGGTGAAGCTTCTCTGTGGCTAATGTCATCATAGGGGCCAGAGTGCAGGGGCTTTGTATGGATGTCTCCTGCAGGC
TTGATCTCAAGCCTGCCGTTAGCTGGGCAGTGTCCCCTTTCTCAGTGGCTTCAGGGAAGGGTGTGCTGTATATATTTGT
GTTTGTATATACAATACGTTTGGTGCACACAGACTAGCACTGGGTTGCCCAGTGGTCAGCGTAGCTGCTGTGCAAGGAT
AGCGTATAAATTCATGATGAGCTCCACTTAAATGAATTCTTTTATGTGTATAGGTGTTTTAACTGTGTGTATGTCTACA
TCATGTGCACTCCATGGAGGTCAGAAGAGAGCACTGGGACCCCTGGAACTGGAGTTGCAGTTGGTTGTGAGCATTGTGG
AGGCTAGGTTCTCTAGAGGAGCAACCAGTAAGAGCCTGGGTTCTCTGGAAGAGCAGCCAGTGCTCTTAAACATTAATCC
ATATCTCCAGGCCAAGTGTCCCATAGGGTTAACAAAGCAGCTGGCATCAGAGGCAGGATGACAAAAGTTGGTCACCGGC
CTCCACGTGCATGTACACACATATGCGCCTGCAAGCATCCACATAGAAACACACACACACACACCCCACATGGGCAGAG
AAAGGATTTAGTGCCTACCCAGCACTGAGCCTCAGAGACCGCTGTGTTCTCATCACTGTCACTTCCACTGACCTCCATT
CATTCATAAAGACAATTACTATCTGACGGGCAAGAGACTTGTCAAGCACCAAGCACGAAGAACTCGTTGAAATATTTTA
CAGACTATATATAAATATACCCATTTTAAGCATACAGATTTATAGATGCTAAAATCTGAAACACCGGGATAAACACAGT
CCCTGTCAACATAGAATTTCTTCTTCATCCCCAAGAAATCCCATAATCCCCCGTGTAACGTCTTTCCCCACCCCCAGCC
CAGCTCTCAGCCACTGTAGGTTTATTTTGTTCTTATGGAGAACCATATAAGAGAATGAGTATAGTGTGTGTTCTTCTGT
GTCTGGCCTCTTGAGCTCAGCATAATGGGGTAGGGAATTGGTAACTGGTTGTTTGGAGTGCTGAGTAGTATTCCATGGT
AGATGTAGACTACACTTTGTTTATCCTTTTGTCTACTGAGAGATAATTGGGTTAGTTGTAGTTTGGAGGGTATTGCAAG
CACAATTCTTTTTTTTTCTTCTTCTTCTTCTTTTTTGGTTTTTCGAGACAAGGTTTCTCTGTATAGCCCTGGCTATCCT
GGAACTCACTCTGTAAACCAGGCTGGCCTAGAACTCAGAAATCTGCCAGCCTCTACCTCCCAAGTGCTGGAATTAAAGG
TGTGCGCCACCACTGCCCAGCTCACAACAGACAATATTTTAAAATATTTTTTCCTAATATGGAGCTTGGTTTTTTATTT
TTTTAATAGTGTTTTTGTTGTTGCTGTTTTCAAAAATGGCTTCTGACTGTGATAAAATCTAAGTGACCACTCTTTCTTT
CTTTCTTTCTTTCTTTCTTTCTTTCTTTCTTTCTTTCTTTCTTTCTCTCTCTCTCTCTCTCTCTCTCTCTCTCTTTCCT
TTTTTCTTTCTTTCAGGTTTACTTATTATATATAAGTACACTGTAACTATCTTCAGACACACAAGAAGAGGGCATCAGA
TCTCATTACAGATGGTTGTGAGCCACCATGTGGTTGCCAGGATTTGAACTCAGGACCTCTGGAAGAGCAGTCAGTGCTC
TTAACCACTGAGCCATCTCTCCAGTCCAGACTACTTTTTCTTTTATGTTTGGTTCTTTTTATACACCAGGGAATCCCAC
ATTCTTTTTTTTTTAAAGATTTATTCATTTTTATGTATATGAGTACACTGTAGCTGTCTTCAGACACACCAGAAGAGGA
CATCGGATCCCATTACAAATGGTTGTGAGCCACCATGTGGTTGCTGGGAATTGAACTCAGGACCTCTGGAAGAGCAGTC
AGTGCTCTTAACCATTGAGCCATCTCTTCAGCCCCCACATTCTTTTTTTCTAAAAAATTTATAGTTTTGGCTTTATATT
TGTTTTTATGCTTTTTCTTTCAGTTTTTGCATATAGTCTTAAGGTAAGAATTGAAGTTCCTTTGTATTTCTGTTTATAG
ATATTCAGGTGTCTTGCTTCATCTGAAGAATAAATTATATTCTTCTACCCACTGAGCTTTTAGGATTCCTTATTGTTTT
TTTTTTCCTGAGCTTCTATGTGGTTATATTTTTGCATTGATGTGTTGGTTCGTTTTAAAGTCAACTCTAAAACCAACAT
GTATGGGTGTTTTGCTTGCAAGTATGTCTGGGCATGCAATGCCCACAGAGGCCAGAAGAAGGCATCTGATCCCCTGGGA
CTGGAGTTAGACATAGTTGTGAACCGCGATGTAGATGCTGGAAATCAAACCCCAGTTCTCTGGAAGAGTAGTCGAAGCT
CTTAACTGCTGGGCCATCTCTCCAGCCCTGGTTTTAGGTCACCTAGATAAAAACTAGAGTTATTTGGGAAGAGGTACTC
TCAACTGAGTTTGGGTAAAGGCAACTCTGTAAAGCATTTTTTTTTTTTAGAGAAATTAATGATTGATGTGGGAGGATTG
AGCCCACTGTGGGCAGTACCATCCCTTAGGCTGATAGTGCTGAGTTGTATATATAGGAGAGCAGACTGAGCAAGCTGTG
AGGATCAAACCAGTGAGCAGTATTCCTCCACAGCCTCTGCTTCAGTTCCTGCATCTTGCCTTTAGGTCTTGCTCTGGCT
CCCCTTGATAATGGACTGTGATATGGACATGTAAGCCAGACTCACCCCCAGTACATGTCACTTTTATTCCTCAAGTTAT
CAAAACAATCAATACCCCGATTAAGACAACTCTCTGATCTTGGGCCTTGGATTTACAGCATATCTGAAAATCAAATAGA
ACACCAAGTCTGCCAACGGATATATCTTTAAAAAGATTATTTGTCTATTGTAGGTCCTTAGCATTTCTATATAAATATT
GTTATTAGTTTGCCTATTTCTACAAGGAAAACTCGTTTGAATTTTTATTAGAATTTCATGGGTCAAATTGGAAATAAAT
TGATTTTTTAGCAACATAAGACCTTCCAGTCCATTAACATGGTATATATCCTCATTTATTTAGATTCTCTTTATTTTTT
TTACTAAAGATTTCCCATAGTTTTCAGTGAATAGGGCTTGAATATACTTAGTTTAAAATGTCTCTAAATAGTCCATATT
TTTTTTAGACGACTTAGGGTTTTTTTTTTTTCCCAGATGTTTATTGCTGATGGACAGAAACAAAATATTGTATGTATTG
ACTTGGCCTTTGGTAACCCTTCTCAATCCTGTTATTGCCAATAGTAACTTTCTAAGATGAACTCCTTAGGATTTTTGAT
GATTACTGTTTGCAAATAAAGACAGTTTTACTTGTCTGTTTCTAGCATTTTTCTTTTCTTTTCTATTGAAATCTAAGTT
GGAATACATCCCTCTCCAGTTCCAAGCCCTTCAGTGATTTCATTCCAGTAATGTATAGTCAAAGACCTTATACTAGGTC
CTCTGTCTAGGCTGAGGGGACCCAGTGACACTCCCCCCCTGCAAGAACTTGACTTTGGGGTGGTGGTAGCTCAGGCATG
AAAACTGGGGGAGTCGTTGGGAGCAGACAGATAAAGGAGGCCATAGTCTGCTAGTATGGAGTTGCCCTGCCCTCTGGGA
AATGTATTGTTATGCAGCTAAACTCTATATCCTCCTTGAGGTAACAGGACTGTCTTAGTCAGGGTTTCTATTCCTGCAC
AAAACATCATAACCAAGAAACAAGTTGGGGAGGAAAGGATTTATTCAGCTTACACTTTCATGTTGCTGTTCATCACCAA
AGGAAGTCAGGACTGGAACTCAAGCAGGTCAGGAAGCGGGAGCTGATGCAGAGGCCATAGAGGAGTGCTACTTACTAGC
TTGCTTCCCCTGGCTTGCTCAGCTTGCTCCCTTATAGTACCCAGGACTACCAGCTCAGGGATGGCACCACTCAGAATGG
GCCCTCCCACCCTGATCACTAATTGAGAAAACGCCTTACAGCTGGATCTCATGGAGGCATTTCCTCAAGAGAGGCTCCT
TTCTCTGTGACAACTTCAGCTTGTGTCAAGTTGACACACAAATCCAGCCAGTACGAGAACCTTGTAACTAGACACAGAC
CAATCTCAAGGGAACACTGGTCACACAGCCAGTTCTCACAGATCAGGCAAGTGAGTGATGGGGACCTCCTTCCACCCCA
GGGTTTGTATATTCCAGCAATCAAGAGGACAGCCATCTCTAAGTGCTCCTGGAGTGAGATGGCCCTTCAGCATGCTTGG
CCTGGGTGAGTCTCGAGGTGGGTGTCAGCTGGCACCAACTGGGAGACAGGGGCAAACCTGGTACTGCCATACCTTCACA
CTGCATTTGGGGGAGGCCTTGCCCTGAATGCCAGGCCCACAGTGATGGAGACAGCCCTGCCTTTTATGCTCATCCAGTG
AGGAGACAGAAAGAAAAGTCACCACAGACATTGGGTGGGCAGGTACTGGGAGAAAATTACTCACCTGGTGTACTGGCTA
GTGTTGTGTCAACTTGACACAGCTGGAGTTATCACAGAGAAAGGAGCTTCAGTTGAGGAAATGCCTCCATGAGATGCAA
TTGTAAGGCATTTTCTCAATTAGTGATCAAGGGGGAAAGGCCCCTTGTGGGTGGGACCATCTCAGGGCTGGTAGTCTGG
GTTCTATAAGAAAGCAGGCTGAGCAAGCCAGGTGAGGCAAGCCAGTAAAGAACATCCCTCCATGGCCTCTGCATCAGCT
CCTGCTTTCTGACCTGCTTGAGTTCCAGTCCTGCATCCTTTGGTGACCAACAGCAGTATGGAAATGTAAGCCGAATAAA
CCCTTTCCTCCCCAACTTGCTTCTTGGTCATGATGTTTGTGCAGGAATAGAAACCCTGACTAAGACAAATTGGTACCAG
GAGTGGGGTATTCCTGTGACAACCTGACCATGTTTTGGGGAGGACTGTGGAAGGACTTTGGAACTTTGGGCTTGAAGAT
CCATCCATTGTTAAGACCTCTGTCGGATGTTGTGTAGGAGCTTGGAAGATAATGTTGAGAACACTGCAGAAGTTGGAGC
TCTGGTGTGTGAAATTTCAGAGGGAAAATTAAAGACTCTTTTCAGGGCCATTCCTGTTTTGATTGTGAAGATTCTGTAG
TTCTGGTTAGCTGGGGCTGAAGAATCAGCTGTGATTAACAAGATACCAGAACTACTAAAGCAAAAACTTTGCATTACTG
GGACTATTGATGCTGGTTAGCTGGAGCTAAGAAATTAGCGGTGATTAAGAAGAGACCAGCATCATTGAGGTGACATCTT
CTGGGAAGTATTTTCTGAAAGCACAGTGGCTGTGTTCCAGATATAGCCAAAGTTGTACCTTGTGCTGTGGCTGGACTTG
GTAATGTGTAAGGGTCACCCAGGTGGTACTGGTTTTGAAGGCATGAAGGAGTTGAGCAGAGAAGCTGAGGCTTGGCACT
GTGAGAGGCCATGGAAGGCCATTGGTGAAAGTGCAGCCTCAGTTGCAATTGATGGCCCAGGACTGAAGGGGTCATGCAG
TGTTTTGGAGATGCCAGTACCATGAGATGACCACCAAGAGCAGCAGCAGCAGTTGAGTACAGGCATCTGGAGCCTAGAG
GATGACGCCTGTGCTACAAAGGGCATGGCTGGAGAAGTGACCCAAGCCCTTGGAGGAGCCCAGAAGATCGTGAGTTGGA
TCCCAGACATTGGACGGTTGGAGATTGACTTTTGCTTTTGATTGTGACTGTGCCCTGATATTTTCCCTCTTGAAGGAAG
AAGCTGTTTTAGTGGAGCCCACAGTTAAGAGACTTTTAATTGTAAAAAGACTTTGGATTTTGAAGGAGATGGATATTTT
AAAGAGATTGAAATTTTAAGAATATGTAAAGACTGTGGGACGTTTAAAGTTATTTAGAATGGGGAGGAATAGGATTGTG
GGGGTTGAGGCTTACTAGTGATGTTTTTGTGTGTCAAGTTGACAAGGGGTCAATTGTACTGGCTAGTGTTGTGTCAACT
TGACACAGCTGGAGTTATCACAGAGAAAGGAGCTTCAGTTGAGGAAATGCCTCCATGAGATGCAATTGTAAGGCATTTT
CTCAATTAGTGATCAAGGGGGAAAGGCCCCTTGTGGGTGGGACCATCTCAGGGCTGGTAGTCTGGGTTCTATAAGAAAG
CAGGCTGAGCAAGCCAGGTGAGGCAAGCCAGTAAAGAACATCCCTCCATGGCCTCTGCATCAGCTCCTGCTTTCTGACC
TGCTTGAGTTCCAGTCCTGCATCCTTTGGTGACCAACAGCAGTATGGAAATGTAAGCCGAATAAACCCTTTCCTCCCCA
ACTTGCTTCTTGGTCATGATGTTTGTGCAGGAATAGAAACCCTGACTAAGACACCTGGTCTTGGGTTATAGCAAGCAGT
TGGATAAATCTTCCTGGGAAGTGATAACCCAATTTCTAAGTTTAAAAAAAAAAAAAAAAACCATGGTCGGAGAGTTCAC
TGATTCCTTTAAGGGTGGAGGCAGAAACTTTGAGGATTTCCTGCAGGTCTGATAAGCTGGGCTTTGTCATCCACTTCCC
ACCACACCTGTGCCAGTGTTACCTGGCTGTCCTGAGTGAGTCAGAACCTTGACTCAAGCAAGCCATTCGCAGCACAGTG
GATCAAAGACTGTCCTGAGGAGTGTTGGGCATCCACTGTGTGTCCCATATACTGTTCTTGGAATACACCTTTCCAGAAT
TCTCTGTAAGGCTGGCATCGTTACCTTTGATTTAAAGTATATGAACCGAGGCTCAGGGGGTCTCACTGTGTGCCAGGCT
AGCCTAAAGCCAAGCCTAGATTTGCACACGTGTCTGTCAGCCTGCAAAGTGTGAGAGCCCTCCTTTCTTCCTTCTTGTT
CTCAAAGGCGTACCCCAAGTCAATAACGGTGACATTTGTGATGGCATCCTCCTGGCCTTGGGCCTCTAAGTGTGACAAA
AGCCCAGGCTGTGACTCAGACAGAGGCTTCCATGTCTGCCCTTGGGCTTCAGTGCCCTCAGCCCCCCTCAGACATTCTC
ACTCCCTGAATATGAAGTTCAGTGGTCAATGTTTACAGTGAGTAAGACCATGAGGGTGAGTGAGCCCGTGTGCTAGTTA
TATGGAAACAGACGGAATCATCTCCTCCACCTCCCTGACACAGGTCCGGCTTGGGCTTCCCTTAGCACCACTAACAGGC
ACAGAGAAAGACAATTTCTTACATGCTCGCTTTAAACTGCAAGGCAGTCAGAGCCACTAACCCCAGGCTGCAGAAAAAA
AAAAAAAAAAGTAGGCTCAGATAGACAAAGTTATCTGATCTGGGATCAGACTGACCTGAAAGCCCACCTCATATTCCTG
CACCCCCACCTGCTTTTCTCTATCCCTTTCTGCGATGCCGTTGGTGGGAGCCAGGAGGCAGAGGCTCAGCAGCTCTCCC
AGAGCAAACAGCCCCAACCTGGGACTCTTTTCCAGACTGAGGAATTCTGCCGCTTGCTGGGCTACACCTTACCTGGAGA
AGAGCCTTTTAGCAGGACATGGTAATGTATGGCCCACGGTGACAAAATTTAAGGTCATAAGGTCACTGCAGTGTCATGC
CCTAGCTCCTTCATGTCACTTTTTCACATTGCCCATCAGGAGGAGTTGGGGAACAACCAGAGCCCAGAGGAGATCCTCC
ATCCTGGCTACGTGCTGCAATAGCCAGAAAATTTTTATAACTCATCAGTGCCTGGGCCACACCCCAGACACCTAGAGCG
GGCCAGAAATCTTGACAGGCTAAAAAGTCTTCCTTGCACCACTGACTGTGTGTGGCAAGCCACCCAGGTTGAGGGTCCC
TTGCTACGCGGTGCAGTCTCTGCTGTCCCCATCCTGTCCTCCTGAGTAAGCTTGCTCATTGTAAGGGCTCAGAACTCAG
CATAAGGACAGAGTGGACCACTGATATCTGCTCTCGATGACTAGGTCATCCAGGGCACTGAGGCACCTCTGAGAGCTTT
GCTCTGAGAGATTTTCTCATCCCTGGAAGCTGAGGAATTTTGGTACTTTGGACCACTTTCCTTAGAAATCTGACTCTAT
AGCTCCCAGAGGTTGGCCAAAGGTAGCTAATTTTGTAGCTATGGAGCCAAGGGCCAATAGAGTGGGGCACTTCCAGGAA
GGCTCCCAAGGCAAGCCTTTCTGGCTCCTATTGATTCAACCTCTGCCATCCCAGCTTCCCAGGCAGGCCTGATACTGCA
GTTGGGTCCAGGAACTAAGAGATAACAGGTAACACACTTAGAAATGACAGGCATCGGAGTGGCCTGACTGCTATGTGCA
GGATGCAGAGTTGATGGGGAGGCCAGCAGGAGACACAGTGTGTGCATTTGCAGGTGACACAGTGTGCCATTTGGATAGA
AGTGCTCTTGCTTTTTTGACTTCCAGGTTTATGGATCCAAACCCGAGGCTATATCCCCACTCCACCATAAAGAGTCTGT
GAGTCTAGAAGGTCAGAGAAACACTGGGACTCCTCTTTGGGATCTGAGTGCCTCTGGGCGAGATACACCCAGGGTGGGT
GGACCCACAGGAGATTTTCCTAGGGACTTGGTGTCCCTGGCACCCTGTCCATCGTGACTGGGCTGCCACAGTAGCAAGG
GATCAGTTGAGAGTCAGGGAAAGGGGACACTAGGCCTCTGGAGGCGGTGCTCTGCCACCCCTTACACGCAGAGACCTTC
ATCTGGGGATGGGGTTCTTATTGGAGGGCTTCTCCTCTCTAGTGGCAGACTGGAAGGTCACAGGTAGGCACCCAGAAGG
CTCATTTCCTTTCTTCTTACCCGGATGGACCTCTGTATAAACTGACAGCATGACCACTCCAGGTAGGCTTTTATTGCAG
AAGAGAGAGAGAGAGAGAGAGAGAAGAGAGAGAGAGAGAGAGAGAGAGAGAGAGAGAGAGAGAGAGTATTTAGTCTAGG
CTACTCCCCACAGTTACCTGGCAACAGCCAGGTGTGCCTGACTCACTATAAAAGAGGCTGCTCTCCCTCACCTCTCTCT
CTTGCTCTTCTGCCCTTGTCCCCTAACCTTCCTCCCCCTCTCTCCCCATTTCTCCCCCCTCTCTCTCCACATGCTCATG
ACTGGCCTCTTCTCTCAACTCCCCTCCCTATGCCCTGAATAAACTCTATTCTATACTATACTGTCATGTGTGTGGCTGG
TCCCTCAGGGGGAAGGGATGCCTCAGCAGGGGCCCACAGAGGCACCCCTTCCCCCACACCTTACCTCACCTCCACCAAA
CACATTCCGTCTCTCTTTAGTTTTAAAAGACACAACAGAGAGAAGGAGTGGGAGAGTAGAATAGCTATAGTCATCTGTA
AGAGTCCAGAGCAGAAAATAGCAGGGGAGAGAGAATAGAGAAGACATGATGAAAGGAGTAGGGGACAGCACAGAGAGGA
GAGGGAGGGAGAGGAGAAAGAGGAGGGGGGAGGGACAGAGAAGGGAGAGAGGAAGAGGGGGGAGGAGGGAGGGAGAGAG
GGTAGGGGTAGGAGGGAGAGAACATAGTGGGGGTGGGGGTGGGGGGACAAACTGCCAGGTTAAGTTGAGAATGAGTAGC
TGTGGGGGAAGGGAAGCCCAGGAGCTGGAGGAAGTGAAAAGACCGGGAAGCCAGCGTTACCTTTGAAAGGGGTGACAGA
TACTAGCAACAAGGAGCAGACCTGGAGGCCAGCTTGTGCTTTGGTATGCTAATAGGCACCACAGGTAGCCGTTGGTCCC
TTCTGCCACTGGTAAGGGAAATGGCTCCTTTTGGTAGAGGGAAACTAGGTTCACCGGTCCCTGAGAAATACTGGCTTTT
ATCTAGTCACGGGAAATTCTCCTGTGGTCCAGGCTGAGCCCATTTCTGGCTATTTGGACCGCCTTTTGGGAGTTCGGGG
AGTTGGAGTCTCCTCTGGCTCTAACAACTTGCTTGTGCAGCCTGCCCAATTTCCTGGGTATGAGTGTGGATCCTGAGGA
AGAATTTACAACCCTGTTACCAGGAAGAGCTAGAAACTTTGAAACTCTGACAGGGACTTCCTGTAAGCCATGGGGCTCC
TAGGGTCAGCCTTCTAGAGCCTAGAGTCCAAGAACCCTCTCTCAGGCCTGAGCGGTAGATGGGGCTTCATTAAGATTGT
GCTCAGTTAGAGGTCCTTCAAGTATGGCTTGTAGGTCTGTGTCTCAGATACTCAGCCAGGGTTTTCAGGGTCTGATAAT
TTTTTTGTTTTGAAATGGGAGAGGCCCTAGCCCAGGCCTGTGTTGAACTCCTGAGCTCAAATGATTCTCTAGACTCAGC
CTTTGGAGTAGCTAAGACTGCAGGGGTGTGTCATCTCACCCTTGTTCTCTAAATGAGTGAGCTGGGAGGGGCTGAGTCC
CTCTCCGAGCTGCCCCTGGCGCACACCATGTCTGAGTGCCGGCTGACCATACACATGTCAGCTTCTAGACTCCTCCTGT
CACCTTGCAAAGGCCAAGATAATTTGGAGGTCACACGGGCCAGGCCTCCTGTGATGGGCGACTTTCCTCTCTGCCCCCT
CCGTTCCACGTGTAGCTCCTCACTCTCCCAGCCACAGCCATAGCCAGCCAAGGATTGCAAGAGCCTGGTGGTAAATTCC
AACCCAGCTACCCTCCTCATCAGGTCGGAAGTTTACAGGATCTGTCCCCTTGCCTTCCCAGATAGGTGTGCCAAAAGCT
TATAACCTGGAGATCCCAAGGAAGGAATTCGAGGCGGGAAGGGAGGACCAATGAGAAGAGCCCCAGCCCTTTGGTGCAG
GAAGACTGGGACCAATGAGAAGAGCCCCAGCCCTTTGGTGCAGGAAGACTGGGACCAATGAGAAGAGCCCCAGCCCTTT
GGTGCAGGAAGACTGGGACCAATGAGAAGAGCCCCAGCCCTTTGGTGCAGGAAGACTGGGACCAATGAGAAGAGCCCCA
GCCCTTTGGTGCAGGAAGCCTGGGACTGTTCTTCATGAGCAGTTTTAGAGCATCAACAGACACCGTCCCCATTCTGCAC
CGCGTCACATGCTTATGGCTACGGGGAGGGTGCTATGGAGGTCAAGAAGGGAGGACCAGAGGCGGGCGCAAGGATGAGG
AGGCTGTCCCTGACACCCCCTCCATCAGAAGGGTTCTGGCGTGCAGCTCATTGACATTTAAAACATCTGATGTCTTCTG
CTGGGTTGACAGTAATGGTAGTTTTACCCTCTCTTTATACTAATAATTTCCAGAAAGTAAATTTAAAACAGGCCGTCGG
CTTAGGTATAGACGCAGGGCAAAGTGTTCCCAAGTGTGGTTTAAGGAAAGAGGTGGCCTCAGCCTTTGCAGAGTGGACT
TCCCTTCTCAGACAGACGAGAGCTTTAATTCTGCTCAACACATTATTGCCATTTAAAAATTTTTCTTCAAGGTTTTTTT
TTTTTTAATCTGAGAAGGAATAATTATTTTGCCTAAAACCACCACTTTCTACCATGTAAAAAGTCTAACATGAATGGTG
AAATCCGTCTGCAGTGTGCCATGAGCAGCTTTAATAATAACCATCTGCCCAGCCTTGTTCATCTCCAGCTCTCTCCCTC
GCCTCTGGTCATTGGATTACCGGAAGGCCAGCTCTAGACTTGAGGGCTTTCATCTGTGAATACTTAGTGCTCTAAGAAA
TAGGGCTTTCTCTTCAAAACCAGACGTGCAAGCTAATGCAGCAGCCTAGAGAGTTAACTGTGGCTCCTGAGTGTCATCA
GGTTCCCCCCAGTGATTAAACTCCCCCTGCTGCCTCCTTACGGGCACGTGTGGCTGTGCAGCTGTGTGTACCCCAAGAG
CAAGAGAAGAGCATCAATAGTTCTGCGCTATTCCTCTCAGCCACACCCCCTTGAGATAAGTTCTCTCACTGAACTCAGA
ACTCCACTGAGAGTCAATGAGCCTCCGTGTCCGTAAGATCTTCCTGTGCCTGTCCTCTACCAAGCTGGTTACTGGTTAA
TAGGTGTCCACACCGGTGTTTTCCTCTGGGTTCCAGGTTCTAAACTCAGGTTCTCACACTTGGACACCAGATGTTCTTA
TCCATTGAGTCACATTTATCCAGGTCCTACTTGTGTTAGAATGGCGGCTTGTCCGGACAGATCACACCAGGCCAGCACA
GTTCCACAGGGAAAAGGTTTATTGGAGGGGGAAGAGGACGAAGGTGGGCAGCGAAGAAGAAAGAGAGAGAGAGAAAGTC
AGGGGGCTCTGCCTTTTATTTGGGCTGCGATGAAACTGCTCACACAATTGTGCACAGGTGAGGACAGGCAGGTGAATGG
AAGACGGTTGCCATGGCAACAGATGCAAGTCCCTGTTGTCTATGGGTGACATCATTGTATTTGAAAGTTTTCTTCTGAT
GCCAACACTACTAATGGGTTTTCATAGATGATTTGTTGAAACAAAGTTTGGACATCGATTGTGCTTGGTTAACACGCCT
CTTAAGCTTCCTTGAGACAACCATTTTTCTTGTAATATATTTGTTTTTTTAAAAAATCATATGCCCTGCAGAATCTTTC
ATATCTTGACATTGTTGATTGTACCGTCCTGGTGCCTTTCATGTATTCCTCTGGTCTCTCTTCCTGACAACAGTAGTTA
GATTTGGAGGTTTCATCAGACCCATGGTAAATTCTCTGTAAGAAGTCGTTGACAGTGAGATTAGTCTTTATTCTAGCAA
ACATGTCCTTTTTTATTTTTTTATATATTTTTTTATATTTTTGGTTTTGTGAGATATTCATTGCAAAGTTTTTCATAAT
CTCTGGCTTTGAGTTTTAAATGGCATCCGTGGGCCCACACGATGGGTAAAATAAGGGTGCTCACTGCCAAGCTTGACAA
CCTGGGTTTGATCCCTGAAACCCACATGGTCTGACACCTGGAAATTGTCCCTTGACCTCCACCTGTGCACTACAATACA
CACGCCGACACAGTCAAATCAATCAATGTAAAAAAGTGTTGACGTCATCCCAGAATGCCATGGTCCATCATTATGTCAT
GACATGCAAAACAGTGGTTTTTAAAGTCTGCCAAGCACTTCTGAATTTCTTACCTAAGACTCTTCCCTACAGCGATTGG
CTAGACCCTTGGTGACCCTGAAACACGGAGGGCGGAGGAGCTCCAGATAAACCTTTGACTCCGCTTGTGTCCCAATACC
TGTCACAGGCAATTAGTGAGACGGGTGTTAACCTCTGAGTATCTTTCTGAATGCCACCAGGCTAGCCTGCCACCTTTGT
GGGTGCCAGCTGAAGATTTGTGAGGTCCAAATCAGAGGCGGTGATGTTCACACCGATTCTCCCCTCTTCCAAGTCTCTC
CAGGGAGGGATGGCGTAACCCAGGCAGGTACTGTCTATACAGTGGGGTTGTGTGGTAGCCGAGCCACACTGGACCTAGG
AGACCATTGAGAGATTCACCTTGGCTCTGAGTATGACATAGTTTGACCCAACATAATTACTCTATGCACACATAACTAA
GATAGCCAGTCTGGGTTAGGTGGGGACAAGGCTGGAGAAAAGCAGGGGTGCTGGGCTTAAGTCCTGAGCTTCTCGAATT
ATTATCCAAAGGCACAGTTATTTTTAAATACGTTTGATGTATTTTATTCTGTCGAGGTCATTTAGCTTGCTGGTGCTCA
GGCGTCCCGTATATGACCGGTGGGAGCCTTTGATGTGGCTCCTGTACCCTTTTGGTATAGTCACAGTGGTCCTGCATAG
TCTTCTAGTCTGACAAGACCTTCTGGCCTCACCCTGTTCAATTCCTACCTCAGAATTCATATATTTCCTCCTTTCTTGG
CTATTGGTGTTTAGATACACAAAGTAGGTTGAAAGTGTGCTTTTAGGTTAGTATCCGTTCTGTGTCCTAGGGAAATAAG
TATTTATTTTGAAACATAGAACTATTATTTGTCATATTGATATTTTAGATTAAAATTTCAGATTATAGGGCTCAACTTT
AAATTTTTTGTTTTCTTAAAAGTGTGTGTGTGTGTGTGTGTGTGAGTGTATATATGTGTGCATGTTTTGTGAACATGTC
GGTGTGCATACATTTGTATACACATGCATGTGTAGGCTAGAGGTTGATATTGGGTTTCTTCTTCTATCAATGTCTATTG
TTTGTTTGTTTGTTTGTTTGTTTGTTTTTGTATCTAAGACAAGATCTCACACTAAACCCAAAGCTCATCAATGACTAAC
TAGAGTGATGGTTGTCCATCTTTGAGGATCTGTTTCCTCTATGTACCTCCCTTCCAGCCCTCTGAGCCCCCAACTCTAG
GGGTTACATGTGTGAAGCAAACCTGGCCTTTTCATAGACCCTGGGCATCTGAACTCAGGATGCTTGCATGACAAGCACT
TCACCGACTGACCCTTCTCCCCAGCCCCTATTCCTCTAGTTTTTACATAAAAGATAACGTATTGTCCATACTGTCTGGG
CAGTGGTGACACACACTTTTAATCTGAACACTCCGGAAGCAGAGGCAGGCAGATCTCTGAGTTGGAGACTAGCCTGGTC
TACAGAGTGAGTCCAGGACAGCCAGGGTTACACAGAGAAACCCTGTCTCAAAAAAACACAACAGAAAACCAACCAAACA
AAAAAGATAACATGCTGTTGCACGGTCTCCCACAGGTCTGTATCGTTGCTGACCAATTTGTGGTGTTATTTTGAAGACT
GTGTTGCCTTAAGTGGCCAGGCTGGAGGAAGGAGGCCATTGGATCAGGCCTTTGGGGATTATACCTGGCCCTGGTTCCA
GCCTGAGCTCTGAGCCCCCTTGTCATGTGAGAGGTGGTGACTTCTTTGCCCTCAAAAGGAGCCGCTCCAGATGTGCCTT
CCCTACCTGAATAGACCATAATCCCTCTGAAATGGTGAATCAAAATAAACTCTCCCTCCCTTAAGTTGGTTCTCTCAGG
TACTTTGGCCACAGCCATAAAGAAAGCAGCCAACTCGCATATTATATACATGGTCTGCACCTTCCTCTGTTTCACATTA
GATGCCAATACATTGTCCTTCTTTCTAACTCCATCTTGGCTCTGTATTGCCTAGTCATGGTGGGTGGGGGCTATTGGGC
ATTCTCCTGTGTTTCAAATGAGTGTTTCCAGCCTTCTGCTATTACAAGCAGCACTTTAAAGAATGATCTGACGATAAAT
CCGTTTGGATCATTTGCTGGTAAGTCTGGGACTAGATTCTTAGAAGTCCAGTGTAAGAGTGACCAGCGCAGATTCTAAG
CCCAGCCCTAAGGTTACCGGGTCCTGTGAGGTCTCTGAACCTCTTATTGAAAATGAACCTTGCTTGCCTACAGGCGGTG
AATGCTGTCTGGGGGAGGGAGTGCTGTTCATTTTCTGTCATAACCTTTCACAATAAGCCTTTTATTTTATTTTTTTTTA
AGTTAGTTTATAGTTCTAACATCTGTCTCTCCTCCCTTCCCTTAGAAGGATGGTAAATAAGATCTTTGACTTTTGAATT
GTTTTAATTGCACAAGTCATGAGTCATGAATAGATCTAATACAAAAAAAAAAAGAGTAAAGGCTAGTGTAATTTTGTTT
GCTATCATCATCAAATTTCCTTCCACACTGGGCTGGTTAGTTTTATGTCAACATGACACAAGCTAGAGTCATCAGAGAG
GAGGAAGAGCCTCAGTTGAGAAAATATCTCCATGAGGTAGGGCTGTAGGCCAGCTTCTGGCACATTTTTCTTAAGTAGT
GATTGACAAGAGAGAGTCCAGCCCATTGTGGGCGGTTCCATCCCTGGGCTGGTGGTCCTGGGTTCTATAAGAAAGCAGG
CTGAGCAACTCATGGGAAGCAAGCCAGTAAGCAGCACTCTTCCACCGCCTCTGCATCAGCCCCAGCCTCCGGGTCCCTG
CCCTGTTTGAGTTCCTGCTTGAGCTTCCTTCAGTGACGGACTACGATGTGGAAGAATAAACCAAATCAACCTATTCCTC
TCCAACTCACTTTTGGCCATGGTGTTTCCTCACAGCAATAGAAACTGCAGCTAAGACAGACAGGGTTGGGCCTCGCCAC
TTTCTATCACTGAATACGTCATCAAGAGTGATGTGATATTTTTCTTAATATTTTCAGTATATCTGAAATTACTTATTTA
CAGCAATTGAGACGTATGATATAACCTTTTTACTTAATTTTAGGAGAGTACAAAGATCTTTCAATAAACTTTGTTAGGT
TGCCCAGGTTGACCTCAGACTTACTGTGTAGCCAGAGCTGGACTTGAGGTTTTGATCCTCTTGTTTTCACCTAAGTGCT
AGGCTTGCAAGCATTTGTCTCCATGCAGCGGGGTGGGGTGGGGGGTGTGAGGGGTTGGGGAATCTGTCTTAGTCATGTG
ACCAAAGACAACAAGTACGGAAGAGGGTGTGAAGAAATGGATCTTCTGCACACTGATCATAGGAATGCAAAGTTGAACA
GCCATTATGGAAAGTAGTAAGGTAGGTAGGTCCTTGAAAGGTTTAAACATGAGCTAGCATATGGTCACACAACCTCTCT
ATTGAATATACACCCAGAGAAGGTGGAGTCAGTGTGTTTAAAGAGACACCTACATCCCAATGCTAGTTGCAGCACAGTT
CCTAAGAACAACAACAAAAACAAACAAGCAAACAAAAACGGCAGCCATTAACTGATGAGTGGGTGAAGAATGTACAGTC
TGTACGCAGTGAGAAACAGAGAACTATGTCATTTGTCACAAATGGGCCTGGAGGTCGTCATGTTATAGGAAATAAATTA
AGCACACAAAGGCAAGTGTTACATGAAGTTATTCTTACGTAGAATCTATGAAAGCTGCTTTTATAGAAGTTGACAATGG
AGTGGTAGTTATCAGAGTACAGGGAGAATTGGAGAATGTTGATCGGTGGATTGTAAGTAATGGTTGAGAGGAGTAACAT
GTTCTCGTGTGCTATGACACAGCCATGTGACCATAGATAACGATAATGCACTGTTTATTTAGAACCACTACAAGGGATT
TTCACCATTCGCTAGTGAGGGACTAAAGATATAGCTTGTACTGGCTAGTTTTGTGTCAACTTGACACAGGCTGGAGTTA
TCACAGAGAAAGGAGCTTCAGTTGGGGAAATGCCTCCACGAGATCCAGCTGTAAGGCATTTTCTCAATTAGTGATCAAG
TGGGGAGGTCCCCTTGTGGGTGGTGCCATCTCTGGGCTGGTGGTCTTGGTTCTATAAGAGAGCAGGCTGAGCAAGCCAG
GGGAGGCAAGCCAGTAAAGAACATCCCTCCATGGCCTCTGCATCAGCTTCTGCTTCCTAACCTGCTTGAGTTCCAGTCC
TGACATCCTTTGGTGATTAACAGCAATGTGGAAAGTGTAAGCTGAATAAACCCTTTCCTCCTCAACTTGCTTCTTGGTC
ATAATGTTTGTGCAGGAATAGAAACCCTGTCTAAGACAAATTGGTACCAGGAGTGGGGTATTCCTGTGACAACCTGACC
ATGTTTTGGGGAGGACTGTGGAAGGACTTTAGAACTTTGGGCTAGAAGATTGGTGTTAAGAGCTCTGTGGGATGTTGTG
TAGGAGCTTGGAAGATAATGTTGAGAACAGTGCAGAAGATGGAAGCCTGGCTTGTGAAATTTCAGAGGGAAAATTAAAG
ACTTTTTTCAGGGCCACTGTTACTTTGATTGTGAAAATTCTGTGGTTCTGGTTAGCTGGGGCTGAAGAATCAGCTGTGA
TACAAGATACCAGAACTAGTAAAGTGAAACCTTTGTATTACTGGGACTATTGATGCTGGTTAGCTGGAGCTAAGAAATT
AGAGGTGATTAAGAAGAGACCAGCATCATTGACGTAACATCCTCTGGGAAGTGTTTTCTGAGAGCACAGAGGCTGTGTT
CCAGAGATAGCCACGGTTGTATCTTGTGCTGTGGCTGGACTTAGTAATGTGTAAGAGTTACCCAGGTAGTACTGGTTTT
GAAGGCATGAAAGGGTCACGCAGAGCAGCTGAGGCTCAGCACTGTGAGAGGCCATGGAAGGCCATTGGTGAAGGTGCAG
CCTCCATTGCAATTGATGGCCCAGGACTGAAGGGGTTATGCAAAGGAGTTGAGGCTTGGCACCATGAAGAGTGCCTATG
AGAGACTATTGTTAAAGCCAAGTTACAGTGGAAGACAGCAGAATTTTGGAGATGCCAGTACCATGAGATGACTACCAAG
AACAGCAGCAGCAGTGGAGTACAGGTGGCTGGAGCCTAGAAGACAACATGGGTGCTACAAAGGGCATGGCTGGAGAAGT
GACCCAAGCCCTTGGAGGAGCCCAGAAGAGTTGGATCCCAGACATTGGATGGTTTGAGATTGATTTTTGTTTTTGATTG
TGACTGTGCCCTGATATTTTTCCCTCTTGAAGGAAGATTTTTTAGTGAAGCCTCCAGTTAAGAGACTTTGAATTTTAAA
GTACTTTGAATTTTAAAAGATACTGGATATTTTTTAAGGGATGAAATTTTAATATGTAAAGACTGTGGGACTTTTAAAG
TTGTTTAGATCTTGGGGATGAATAAGAAACTAAGGGTTGAGGCTTATTAGTGATGTGTTTGTGTGTCAAGTTGACAAGG
GGTCAATTGTACTGGCTAGTTTTGTGTCAACATGACACAGGCTGGAGTTAACACAGAGAAAGGAGCTTCAGTTGGGGAA
ATACCTCCACGAGATCCAGCTGTAAGTCATTTTCTCAATTAGTGATCAAGGGAAAAAGGCCCCTTGTAGGTGGGACCAT
CTCTGGGCTGGTAGTCTTGGGTTCTATAAGAGAGCAGGCTGAGAAAGCCAGGGGAGGCAAGCCAGTAAAGAACATCCCT
CCATGGCCTCTGCATCAGCTTCTGCTTCCTAACTTGCTTGAGTTCCAGTCCTGAAATCCTTTGGTGATTAACAGCAATG
TGGAAAGTGTAAGCTGAATAAACCCTTTCCTCCCCAACTTGCTTCTTGGTCATGACGTTTTGTGCAGGAATAGAAACTC
TGACTAAGACATAGCTTGTTGACAGAATGTTGGCCTAATATTTGTAAGAACCTAGGCTCAATCCTCAGGGAAAAGGAAA
GAAAAAACAGAAATGACAAACATTTGTGGTGATACATATGTTTGCCCCAATTTAAAAAATTGTACCAGGCTGTGGCTAT
AGAATGTTTGCTTAGCACATATAAAGACCTGGGTTTTGATCTTCAGTGTGTGTGTGTGTGTGTGTGTGTGTGTGTGTGT
AATCCATGGGTTGAAGACTCATACAGTTCCCCTGAATATGGATAATTTTGGTTCTATTGGTTTAAAAGCAAACATTTTC
AAAACACAGGTGGTATAAAAGACTAAGTATTTGTATCAGTCCTGGGTAAAGTGCAAGGCTTTGTTGACACATAGACCTG
AGCCCCATTCTTGCCTTCTCATCTGTTGGTCAGGTGACCCTTGGGGAAGCATTATTGGGAAGGGAGGGGTGTGTGAATA
CATAGAAAGCCCATGGCTGAGTCTGGGAGTCATTGCCTGGGAGAGACAAGGACCACCAGACTTCCTCGTGCCAAGCACA
CACGACCCTTTTGTATTCAAGGGTCTGAGTACAGGAGATATTGAGGCTAACACTAAAGGACTAGGCCATAGGTCAAATA
ACTTGCCCCAGTTTCCCCTGAATTTCTCTGGTTTTAGTTCTGAAAGTCCCTGCTCTGGGAACCCCCTCTGTCCTGAATA
AACCACAACAGATGGCTTCCTTATTGGAGAAAGAGACCTGGATTGAGGGGTCCTGAGACCCCTCAACCAAGTTGGAGGC
AGCTACCTCACCCTTGTCAAACATTCATGTAGCCCAGTGGCTCTCAATCCTTCCTAATGCTTCGACCCTTTAATTACAG
GTCCTCATACTGTGGTGACCCACAACCATAAAATTATTTTTGTAATGTAAATATCTGTTTTCTGGTGGTCTTTAGCAAC
CCCTGTGAAAGGGTCCTTTGGGCCAAAGGGGTCATGATCCACAGGTTGAGAAACACTGACATAGCAGAACAAAACAAAC
ATAGCTATTGTTGCCGAGGGGGCTGGAATTGTCTTGAAGCTAAAGTGGAGCTATCACCTTGAGGAACCAGTGCCTGGTT
CCTTTCTCCTGTCTGTGGCTGGAAGCAGACTTAGAAACGGTCTCTGTGGGGTCTAGGGAGATAGTTGTCTGTAAATTGT
TTGCTTTGCAAGCACAGGGATTTGAGTTCAACCCCCCAAGCCCCTGTAAACATCTAGTCTTGGTGGTGCACACTTAGGA
TCTCAGTGTTGGGAGGCAGAGACAGAAAGGTTCCCTGGAGATCACCGGCTAGCCCGTCCAGCACGTGGCAAGATCCAGG
CTAATAAGAGACTGTCTCAAAATAAAAGAAAGGTGGATAGCATCTGAGGTATACCAGTGAGGCTGCCCTCTGGCCTTCG
CATGCGTGCTTACATGTGTGCATGCCTGTGGACACACACACACACACACACACGGCACATGGATCTTTGGCCTCTTGGT
AGCAATATTAACTGGAAAGACAGCCAATCTCTTTCAGGCTGACGGAGGCTAGTATTTTATCAGTAATAATGGTGAGGTA
TGCATCAGCCTTTAGCTGTTCTGAATTATGGCTTTCAATATAAGAAAAAGCGATCCCTGGAACTGAATTTTCTCATTCT
TATTCACTAGGGGTTAGTCATGCCTTCAGAATGGCCAAGTGAGAACCTTGGTATTTGGCAGCCTGGGCTTCAGGCCTCA
AGGCAGGAGTAACCATGCCAGACTTGGGTCATTGATGGAGTTTCTCCACTCTGATTATTTCATCATTTTTATGAAATCA
CAATGTCCTTCCACGGATTATTAAGTGTCCCCAAGATTGCCAAAAAGGAGGCTACTCAGCCAGAAGGTTGTGAGTTGTA
CTGGGACCTCACTATGTCCCCAACATCTTGAGCTTCCAACTCCCTGCTCTTTTACTAGAAACCAAGGAACAGGCTTGGG
TGTGGCTCCTCAGACCCAAGGGGGGGATTTATAACTTGAGTCATGCATTCTAAATCCTATTAGTTGCCTCTGAAGTGTA
ACTTCACCTTCCAAATAGCACAGTCAACCTCCAAAGCCTGGGTTTTCATCCCTGTGAATGGGAAATGAGTGTAGTCTCC
TGTGGCCTATTACACACCCTTTGTTCCCTTTTATTTGACCATCCACTCACATTTCTTCCCCCTCCAATTTCAAGTGCAT
TTGTTCTGCCCCCACCCTCACTCTCCTGTGGGGATGTGGGACCACTTGTGGCTTGGTACCCTCCTGACACTGCATAGAA
CCGATTTCCTTCAGAAATGAAGCCATTCCACCATCAATGACCCAAGGTGGTCCCTCTCTTACTACTTAGACACAGCCCA
GGGTAGCACCAGGCACAGGACTGCTCACTGGGTCTTTCTCTCCAGAGACCTTATTCTCCAAGACAACACCAAGAGGGCT
GAGCAGTGTTTACACTTGAACCCGCTCAGTATAGCTCCCGGAGTGGTGCAGAAGCAGCCTGTCTGCAGGCTCTGACCTC
CTGGCGGCTACCGTGTCTCTTCCACTCTTAAGGCTCTGCACAAAGACTTCCAGAGCCTGGGGTGTGCTGGGTACTGGGG
AAGGTGGGCGTTGCTGGGTGAAGAAAGTCTGTGGGCAGCCATGATTGACGACTTGCCCACAAAACAGCCTGGCTCCTCG
GAGCCTGGAAATTGAAAAAGGACAATTTTTTTTTTTTTAAAATTAAGTGCTTCTTTTCTTTATGCAGTGTACAGCCCGA
TACAAAACACAAGGCACATCACAGTAAAATGTTATCAGTCTTCTGTTCCCATCCCCCACCCTCATCCTAGTTTCCTACA
GATTCGACTCACATCCATGAGAGTGAGGGGTGAAGTAATGGCTGGGAGGCACAGCCTCCTCAGGCATCTTCCTCGTGCA
GCCCTTGTGACCACAGCTGACATTTCTCGGTGGTTCAGATAGAAACAGGTAGGTATGAGGTTTTGTTGGAAGAAGCAGG
GTCTCACTGGAAAAAAATTGTCTGGAGGTGAAAGGTGATCTTGGGCACTGGTGGGCAGGAATGGCTGCAGCCTGTGAGT
TGTCCCATGGGTCTTTAGCACTGCAGCCCTTTCTATTGGGAGATGAAGTAAAAGCCACACAGAGGTCTCAGTTGAGGAT
GTCTTTGTGATGTGTGCATGTCTTAGTCAGGGTTTCTATTCCTGCACAAAACATCATGACCAAGAAGCAAGTTAGGGAG
GAAAGGGTTTATTCAGCATACGCTTTCACACTGCTGTTCATCACCAAAGGAGGTCAGGACTGGAACTCACACAGGGCAG
GAACCTGGAGGCAGGAGCTGATGCAGAGGCCATGGAGGGGTGCTGCTTACTGGCTTGCTTCCCCTGGCTTGCTCAGCCT
GCTTTTCTATAGAACCCATGACTACCAGCCTAGGGATGGCACCACCCATAATGGGCTAGGCTCTCTCCCCCTTGATCAC
TAATTGGGAAAATGCCTTACAGCTGTATCTCATGGAGGCCTTTCCTCAAGGGAGGCTCCTTTCTCTGTGATAGCTCCAG
CTGTGTCAAGCTGACACACAAAACCAGCCAGTACACTGCGTGACGCTGGTACCTGCTGTCCTGTAAATGGAGTCAGGAA
AGCAGATCTTTTCCATCCTGTTAGCACAAAGGCCCAGCACACAGTAGGCATTCTGGAAACCTCTTTCTGCCCATGCCCA
CAACACAAATACTGCAATCTGGCCAACTATGGGATTGCATCCTTGCCAGTCCTTGCTCAGGAAGCAGGTGTGTGTGTGT
GTACTGCTAGGTATGAGGGCTACGGCTGTGGATGACATAATGTCCAGTCCTAGGGAAGCTTCCGTCATGGGGAGGAGTG
TGATCAATAGCCAGATAGCTGTGTGTCCGCAGTGCCAGTCCACAAATGGCATAGCAGACCAGGATGCAGAGTAGGGGTA
CCTGCCATTAATTTCATCAGGAGGGACTAGGTAGGCCTCTGAAGAGGACACGAAGGAGGTGGGTAATGGGCAGGCCATG
TCTGGGAGCTGATCCTAACTAAAACCATCCAACCAGAATGGCTGAATGTGCTGGGAGAGGGGGTGGCAAGAGTCTGGGA
GCAGGAAAAAGGAACCTGGAGTGCCAGGAGATGGAAGAGCAATGGGGCAGAAGTATGGGGGTACAGGTAGGGCAGAAGT
GGGGGGCTGGCTTTACTCTGAGATATGGGAAAGGTCTCAGGGGGCTACAGCATCCCAGGTCAGTGACAGCATCAGACTT
GAGGACAAAGCCATTTGCTCTGCAGCTGTGTAGCGGCTGTAGAGGTTGAGGGCAGACACAGAGAAACACTGAAGAAGAG
GCCCCAGCAGTGACTGGGCCTGGGGAGGAGAGTGCCTTGGCTAGGGTGACCCTGGATGTAGTGAGGGATGATCTAATAT
GGGCAAGATCCAGAGGCAAAGCCAGATGGATGTTATGATGAGTAAGATGTGTCTTATCCACCCATCCACCCATCCATCC
ATCCATCCACCCATTGTGACAGACATTGGTGGGACCTCAGACTTTGCCTTAAGTGCCTGAAGGTACAGGGAACAGCAAG
ACCAGCCACACTGGTGAGGTCTGGGTTTGACTGAAAGTCTCCACAGCAGTTGCATCTCAGATATCCTGTATATCAGATA
TTCACATCATGATACATAACAGTAGCAGAATTACAGCTATGAAGTAGCAATGAAATAACTGCATGGTTGGGAGTCACCA
CAACATGAGGACCTGTATTAAAGGGTCACAGTGTTAGGAAGGTTGAGAGCACTGTTCTACGTCAACTTCAGATCTCCAC
AGTATGCATATACATGCATGCATGCCATACATGCACGTATTTTACACACACACACACACACACACACACACACGGAAAA
GGAAAAAAAATCTCAAAGAATTGGTCAAAGCAGGCTACTTATGTGTGTTCTTTACAGTCTAGAGTCATGATGGGGTCTC
TCAGAGGAGAAGAGCAATAGCCCTCCCAACCCCCGTAATTGGGCAGCTGATGTTGCTAGGTAACCAGACTCAAAAATGT
GAGCAATAGCCCCTTTCCAGCTCCTCAGTGAAAGATGGACTGTGTAGAGTCCATGAGAGATGAGAGAAGGAACCTGCTC
AGGACAGCCAGCACTAAGGGCGATGCTTACATTTTAGCAGGCTCCAATGTTCTTAAGGACACTTCGCCCATGTTGAAAG
TAGGTCTGAATTCCTGAACCTTGCAATTGAGACCTGACTGTGAGTTTACAGCAAAAGTCAATACTTAACAAATATAGCA
CCTGCCAGAATCTATAAACTGCCTTCTGATTCAATATTGAGAAATTTAATTTAAATAGGTCATAAAGAAATGGGGCAAG
AACCTGTCAGGAGTCCTGAAAGATCTCTCTCCTCACAGGAGACCCTGTTGATCAGTTAGGTGAATCCTAGATAGTGTAG
GGACTTGGTGTTTTCTTCATGAATGTGTCTTGAACCACTGTCAAAGTGTGTGTGTGTGTGTGTGTGTGTGTGTGTGTGT
GTGTGTGTGTTGGGGGCAGGGGGTCTTGAAGCTGTGAGGAGGGTTTTTCTGTGGCCATATCAATCATATTGAGAATGAG
TGAAATGTCTGTTCTTGACACCTGAGTGTCTGTCCCCTCTGGGAGCAGGGTGCAAATCACATGATCCAATACAAGAGCC
TTCAAAGTGACAGCTGCCCTTGGTTAGGGCATTGTTCATCTTGTCACCAAAGCTAATTCTTTTCTACACACCCTCACAA
GATCTCTCTGGGTGTCCCAGGGGGATGGCACATAGTCTCTATCCTCAGGGAGTTTGTAGACAAGCAGAGTAACACCTTT
AATGAGTAACACCTTTGCCCTTGAGACGCCCACCTCTAGAACTAGATGATAAAGCTACTTTCTCTCTGTGTGTGCAAGG
TGTACCCTGAGAGGTGTGTATCTCTTGGGCTCCCTTACAGGGTTGCAGACTGAAGCCACCAGCCCGTCTGGCCATAGAC
CTTTCTATCTAGATTTAAGACAGAACAAAACAAAACAAACAAACAAACAAAAAGTTGTTTGTTTGTTTTTTTAAATCCA
TTTTCCTTAGGACCAGCAAGATGGGTGCTTACTGCCAAAGCCTGACAGCATAAGTTCAATGTGTGGGACGCACATGGTA
GGAGAAAAGCAATTTGTAACTTCCACATGTGGTACACGTGTGTGGGGGTGGTGGTGGGAGGGGTGCATATGCACCCGCA
AGTACACACAGAACAAGCACACACAAATCAATAAATGTAAAAATATAAAAATGAATCTCACTTAGAAGCCTAAGTCAAC
AGTGGCTTATTAACATAGTTTTATTCAAGGTCTGTCAGAGAACAATAAGGTAGTCTATTAAGTTAACATAGACACTGGA
AACCCAGGGGGACAGGAAGCAGGCAGTCTCAGACCAGCCTAGACCTGCAGTGCCTTCCCATGTATTGATTCTCTCTATC
TCAGCACTATCAGGTGCCAGGTGCCCCAAAGTCACTGGCTGGCCTGGGCCTTGAAAGACATTTACAGAAGCACTGATCT
CTGCCAGACACCAGGAGTGGCAGCCACTTTGTTTAAGTCAAAAATGTTTCCAGACATTTCCCATGTCCTCTTGGGTTAC
ACGATTACCCCAGTCAAGAACCACTGCTCTCTATTTTGAATGAAAAAAAAATGTATTAATAGGGATCTCTCTCTCTCTC
CTGCCCCCCTCGTGTGTGTGTGTGTGTGTGTGTGTGTGTGTGTGTGTGTGTGTGTTCACTGTTCCCAACACTCTTGGAA
TTCCCATGAATTACATAGACATTTTATTCTAAGGTGGGCCTACAACCAACCCACAATACAAGTTATTAAGTTGGTAGTT
TTCAATGTACTAAAATTTGTATGTAACACATTTGAGTGGGTGTGTTGTTTTTATTACAATTTTTGAGGAGTAGGAAAAT
CTTCATGTTAAAAACATTCAAACCAAATGTTGGCACATCCCTTGAAAGCCCTTTGAGAGTCAGCTTGGAGCCAACGGTG
CTGGCTTGGATCTGAACATGGTCACAGGTTAGAATGGGAGGGCAGCCTAGCCATTGCCCCTGGGACCCCTTGGCTCTGT
CCAATACAGGCCCGAGGAGGAGGAAATTGTATGTACCGGCCTGGGAACCTCTCAGAGGGTTTGGAATTGAAGCAAGGAA
CTCAAGATTGATTTAGGGTGTGAGCCCAAGGGAAAAACAAGCCCCTGGCCCCGAGCAGCCCTAAGATTGAGCCCGAGAT
CCTGGTGACGCAGGGCCAGGAGGTTAAGCCCAGAATAATCATAATGAAAACAATCTGTTTGCACCATTGATGTTTCCAG
AAATTCTTGGGAGCAAGTCCAAGGTGGAGAACCCCAACCGGAAGTGCTGCTGGTTGGAACTGAGAGGGTAGCGGTGGGA
CCAAGGGCCAGAAGAGAGCATCTTCCTCTGCTTCCAAGAGCAGGGGCAGGGTGGGAGGGGAGGTGGCAGGCCCAGGGAA
GCAGCAGCCAGGGGAGGGGGAAGGGGGTCTGTGAGCTGCAGAAACAACAGCTGGATTTAATGTTCTTGGAAACAGTCCT
TTGCTGGAATCAATTTGTTCTTCCTCTTCTGAAATTTCAGGGCTGAATCCTCAGGAAATCTGCCTACAATGAGGTGGGA
CAGCCGACCACAGGCCACCTACCACCACGTCCAAGGGGATATAAAAGACACTAAAATAAGGCAAAGGTCTGACAGGGCA
GCCAACTGTCTGCTTGTATCTCCACAACTTCAAGGGTAGTTCTCAGCAGGAGCCTGGCCTGGGGCTAATGAGGCAGAAA
GAGGCCTTGCTTGATCTTAATGGAAGACACCTTTCTCTCCTCCTCACTAGGGCCACTTAGGGGAAATCCCATTCTTGTC
CTAGGCTTGATCCATTACCATGTATCAGAACTTTTTTAAAAAATTATTTTTATTCTATATGTGTTGGTGTTTTACCTGC
ATGTATGTCTGTGTGAAGGTGTCAGAGCTTGGAGTTACAGAGTTGTGAGCTGCCAAGTGGGTGCTGGGTCCTTTGGAAG
AGCATTCAGTGCTTTTAGCCACTGAGTCATCTTTCCAGCCCGTGTGTCAGAATTTTGTGCTAAGAAAGACCTTGGAAAG
AATATGGTTAGAGGGGTCATTGAGATTTTAGCAAGATGACCTTTCTAGCTACCTGCCCAGGCTTCCTCCTGTCTCCTGG
CTCTGTCTTCCTTTAAACCTGCCAAATTCTTCCCAGTAGGAAAGGTTCAGGGCTTCCAAGCTTGACCACCCTGGGTCTC
CTTGGAGCCGTGGGTGTGGATCTGTATACATTGGGATTGTGTGGGCTCTAGTGTGTGTGAGTCTCTGCACACTGGAGGA
CACTCACCTGCTAGTACATATGTAGTGTTAGGCCCTGGAACTAGGGTAAGGCTTAGGGTGGGACTAGACACTGGAGTCT
TGGGACTCAGGTTGGTTCCTGGAGGACTTTGGCTGAAGCTGATATTAGATTGAAGGCAGGCCGGTCAGATAAGTCATCC
CAGAAAGAATGGCTCAGGGAGGACATTTAGAATGTAACTGGAAGAGGGACTTGGGAGGACACCCAAGGCAAAAGGAGTG
AGCCAGGGAGCCAGAAACCAAGAGCACAGTTGGGAAACACCCACTAGTCCTTGCTGTCTGCAGGACTGGGTCTACATGA
GGCTCAACAGCCATCTGGCCACCATCTGTTGAGTACCCTCTGTATGTGTAGTGTTGTCCTAGGTTCTAGAGGCCAAGCT
GGAGGAGACAGTTGAGAGTTCAGCATGAGTTCCTGCTTTCTGTTTCCCACACACACAGGGACCCTGCTCTGTGGGTTCT
AGAAGAAACGAGACATGGTCTATACCCTCTAGGATTCCCTGAGCTCAGGCAAGACTCCATAGGTCCATTACAAAACGGA
GTGCAGTGGGATGTGGGAGCTGCCAAGGCAGGTTGTTGGAAATTGTACTTACTTTCTTATCCTAATCTTAATCTGCATA
CACCAGGGCTGCAGAGCCAGGTCTGTGCATCATTCTACCTGGGAACGAGCATCCAGACTCATGGGATATACACGTATGT
TTTCCTTGCCTCCAAGGAAGATTCTCAAAGGATGGCATCCCGAACAGACGCCTGGGCCTCGGTGTGCTCTGGGAGCTCA
GTGTTCATATCTCCTTGGGAGAATGTGGGCCTGAGTATCAGAGGGTTTCCATGGAGACCTCTGGCCTGGCCTGGTGTGA
GAGTTCCAGCGAAGGGAAGCGAGTGCGGGCAGGGAGGGGACATCGTTTCTGATGCTTCTGTTGCTGCAGTTGGCTCCAC
CTCCGTCTCTGCCGTGCTTTGCTTTCCAGGTTACTCAGTAACTCTGCACTGAGTCGGGTTGGACTGGGCCAGTGAGAGA
CCATCCGACTAGTCAGTGAGGAGTGTGGTGGAGCTTCAGGCAGTGTTTCGGGAGAGCAGACCTCTTTTCAAGTGTTACA
GCTCTTAGGACAGAAGCTCTTGGATGAACACAGTCGTTCTCACACACCTCTAATCCTTCCAGATAGGATTCTTATATAC
AGTTTGGGGGTGGGTCAGGGTCTGACAACAGGTTCTTCTCAATGGTTTGGTCTGAGAGCTTTGGGGAAACCTTATTTGC
ATGTGGGAGGGCTTGGTGCTGCTGACTGATGGCCACACACCTCTCTGCAGGGGGCTATGGGAGGCTGTAGCTGCAACAG
GAGCCAGGGGCCCAGGAGTCATGATCGAACACCTTCTATCCAATGCAAGTGGAAATCACCACCCACTGGGTCACCAGGG
TTTCAGACCTTTGCTCGACTGGGGACCAGGCTGCCTGTGCACAGTTCACCGCCCCACAGGTGAACAAGACCTAGCACAC
TAACTTCCCACACTGTGGGTGTTATCAGGTCCCAGGCAAAGAGAAATGCAGCCTACCAGCTCCCTTGCTTCATATCTAA
TGGCCGGCCGACCCCTTAGGCAGACCTTGGTATCTCCCTAGGATAGCAGGGGAGATGTTTTGAAAACACCTTCCTTTTC
CTGCCACCACAAAGTCCTCCACAGGACTTGATAGAGCAGGTTCCACCTTATGGACAAGGCTACAGAGGCTGGGGTGGTA
GAGTAGCTATCTCAGGAGCATGCATTGCCACTACTGTCTGCCTCTGGCAACTTCCCCTAAATACAGGTCATATGTGCTG
TGCTCCTATGTGACTGACTGACCCATGTCCAAAACCAACTGTGGGGTTTTCTGAAATGTGCTTTGGAGTAGCCAGCAGC
TTCAAAGACTCCTGGCAAGGCCCTGCCCAGGAAGCGATGAGCCAGCACTTCCCTGGCTATGTGCTTGTGGCTGACCCTG
GCCTCAGGGTCCCTCCCCTCCTCATTAGGGAAGGATGCTGAGGAGGGTGGAGTAGGCTCCGGAGAGAGCCAAAAACCTT
CTGGGAACCATCTTAAGGGAAATGGATGCCCCAGAGTGGCTCTGCCCTCCTCTAGACCTTACCCACCCTCGGCTTCAGC
AGCATTTGGTGCTGGAAAGAATGGACCTTGGTTCCTGAACCATGCAGAATAAAGAGGTCAGTACATTCCAGGGCTTGGC
CTCTCTTCTTCGGTCAAATACTCTAGCAGATGGAAGATCTAGGCCTTGTCCTATCCTTAGATTAATCAGGGAGTTGTCA
GGTCTTTCTCTGATGGACAGAGGTGTTGAAGAAGTGCCTCGTGGTGGGAACTGAGGCTAGACCCCCATATCAAATGCTG
CTGGGCCCAGGATCACTGCCTGCTCCCAGGGCCCAGCCACATGGGACAAGCTCTTGTTCTAGTCCTGTGGCTCTTAGGA
CATCTCTAGCTGTTTTGACCTTCTGTCAGGGCACAAGGGTCTTCTTAAGTCCTTACACCCCTGTCCTTGCCTTCTCCTC
TTCTAGGATCACCTTCTCACCCATCTTTCTCTTGATGACTCCTACACATTCCCTAGCCCAAGCCCTACCCAACTTTCCC
ATGATCCTATGTATGCATGGGAGGGTTGGTCCTTGGGTCCCACACTCTGGGTTTCTGCTCTTTTTGTCTTTCTGGAGCT
TCATACTCTAGGATAATTCCAGTTAGTTCCCTTTCCTAGACAAACATAGAGTGAAGGTGATAGCCCCACTCTAGGCCTG
GGCACTCAGCCCAGGCTGGCTCCGCCCTTTTCTGAGAGGATGGATTCTCTGTCCTTCCAGTTGCGCAGGCCAAAATCCT
GGAGTCATCCTGACCCTCCTCCCCTTTCTCTCATAACCCACACCCAGATCCATTAACCAGCCCTGTCAACTTGGCCTTA
AAATATGAGCAGAGGCTAATTCATGGAAGCAGGAAGCAGCCTTGTGGTACCTGGAATATGGGGTGAGGGGTGGTGACAG
AAAGGCTTAAGGAAACTTTGATGACGTGGAAACCTAACTATCTCAGTAATAGAGATGGTCTCATGGCTGTTATATACAT
TAATGTATACATTACGCTGGGCTCAATGGTTGCCACTAAGTTGTTGGTTATTTAAATCAGAGGTTCTTCATGACCTCTG
CAGTTCTCTGGATAGATCTCTTTCTTCCTCTTGAGCTTGCTGTACTGGCTGGTTTTGTGTGTCGATGTAATAAAAGCTA
GAGTCCTCAGGAGAGAAAGGAGCCTTAGTTGAGGAAATGTCCCCATGAGATCCAGCTGTAAGGCATTTTCTCAATTAGT
GATCAATGTGGGAGGGCCCGGTCCATTGTGGGTAGTGGCATTCCTGGGCTGGTGATCCTGGATTCTATGAGAAAGCAGG
TTGAGCAAGCCATTGGGAACCAAGCCAGTAAGCAGAACCCCTCTTTGACCTCTGCAACAGCTTCTGCCTCCAGGTTCCT
GCCCCACTTGAGTTCCTGTTCCGACTTCCTTTGGTAATGAACAGCAATGTGGAAATATAAGGTGAATAAACCCTTTCCT
CTCCAACTTGGTTACTGGTCATATTGTTTTGTTGCAGCAATAGAAGCCCTAACTAAGACACTTGCCCACATATAAAGTG
GGATCCCCATAGAGTCTTCCAGCCGGGCCATCTGCATTAACCAAGCTTATCATGTCACCCGGAGAGGCTGCAGTGAGTG
GGCCGTGTCTGTGTGACTCAGGTCTATCCCAGGACTCATCAGCCCTTCCGGTAAACGTGGTTTAGACCTAGCTAAGGGA
TTAGTTAGAGATGGGGGTTTGCCAGGGCGCACAGCAAAATCATGCAGGGAAAATAAGGACACTTCCTAGGGTCTCTGCT
GCATGGAGCTCTCTTCAGGCTCCCCCAAACCTCTAGAGCTTGGGGAGAGGATCAGGGACTTTCATCAATACCCATATTT
GACTGATAGGAAAACAGAGATCATCTGGCAAGTCCAGGCATCATCTGGGTACTGATAGAGCTTATACGGGAATGTAGAC
TGGTAGATTCTTTCTTTCTTTCTTTCTTTCTTTCTTTCTTTCTTTCTTTCTTTCTTTCTTTCTTTCTTTCTTTTCTTTC
TTCCTTCCTTCCTTCCTTCCTTCCCTCCCTCCCTTCTTCCTTCCTTCCTTCCTTCCTTCCTTCCTTCCTTCCTTCCTTC
CTTCCTTCCTTTCTTTCTTCCGAGACAGGATACCACAAAGCTGTCCAGGCCTTGAACATGAAATCCTTCTGCCTAGGCC
TCCCAAGTAGCTTAAGTGGCAGACCTATGCTACCAGGCCAGGTCAGGCTACTAGATTCCTAAGCTAGTATCAAGTCACT
CCTTCCCTGACCCCTGTTTCTTTGTTTGCATAACATCTTTTTTTGCATTGTTTTGTTGCAGCAATAGAAGCCCTAACTA
AGACACTTGCCCACATATAAAGTGGGATCCCCATAGAGTCTTCCAGCCAGGCCATCTGCATTAACCAAGCTTATCACGT
CACCCTGAGAGGCTGCAGTGGGTGGGCCGTGTCTGTGTGACTCAGGTCTATCCCAGGACTCATCAGCCCTTCCGGTAAA
CACGTGGTTTCTTTCTAATGTATAACATAATTTACTTATTTATTCCAGGTAGTCTTGCTCCTTGGGAGAAGGTAAGATC
CACACGGCTAGAGGTGGGTGTCTGTTCTGTAAACCGAAGAATTCCGAAAACTTAGAATAGCATCTTTCACAGGCCGGGC
CCAGAACCAGTCCTTAAATAAATGTCTTAGTCAAAGGTCACTTCTCTGCAGAGCAAGGACTTCTAGACGATTCTATTGC
ACTCAGTGGTTCACTCCAGCACTAGCTGCTTTATCTCACCCCCCTTAATCCTCTGTTTTTTGACCTCCAAAATATGGTT
TCCCAGAATTTTTACAAGTAGTGAGATGCCTAGCAGGGGCCAACTGTAGGGAGAAGGGTCTTTGTTTAGCAAATAAATG
TACAAAGCAAAAGCAGGGCAGGCAGAGGAGTGGCGGGCTTGATTTAGGTGTGAAGCCCTCAGATGAGGTATTTTCTTCT
CTCCTCGTAGGAAAATGTTCCGGTTTGAGACCCAGGCATTAAAACTGTTCTGCAGTGGGAGAGGATACACGGCACTCAA
CCCTGGTGACCCTCACTTGCTGGCAGCCAGGTTTGGGGGTCAGCTAGTTCTTTGAGGACCTTTGTCTTCCTCTCTGTGA
AATGGGACATGAATGTATTCTCCATTGACTTCAGAACTGATAGGAGGGTGAAAGTGCTCCTGAGAACACTCTATGAGCA
CTGTGGGGCTGTGCCTGGGGCAGGTGTCTCCAGCTAACAGCAGGCCCACAATGCTGCCTGCCTGGTTTGAACAATGTGC
TTGCTGCTTGGGCAAGTCGTGTTCCCTGGAATTCATATGTTGAACTCTAACCCCCCAAGACACTCCACAATGTTTCCAT
AATTGGAGACAGGGTCGGTCATTAAAGAGCCAGAGAAATTACAGATAGGCCATTAGGATGGAATGTAATCCAACCCCGA
TGTTCTCCCTGTAGGAAGAGGAGTTTTGACATAGATCTTCACAGCTAATATCAAGATAGAAAGGTGACCATCAGTAACC
AAAGAGCAAGAGAGACACCAACACCACTACCATCACAACCACCACCAAGAAACCTGCCCTATCAATACCCTCACCTTTC
AGCCTTCCCAAGGCCTAAGCTGTCCAGTCTGTGGTACTTTGTCACAGCCGCCAGGAAACCAGAGGAGGGACTGCCTTAC
TCTTCTACCATGCAGATCCCCATACCACACATTGCTGCCAACACCCCATCTCCAGGAACCACTCTCCTCCTGGATTCCA
GAGTGAGGCAATCGCAAAATCCCTCTTCCTTCCACCTGCCTAAGGTCTTGTGTAGCCACACAGAGCTGTTAGGGTTGGG
GACTATTTCTGCACTCAGTGAATGCTGCATCCTGAGGGATGCAGGGACTCTCAGGTCACCCACAGGGGAGGGCAGGGCA
TGAACCAGCTGCCAGGCCTGTCCTGCTTTCTCTACTCAGTAGGCATCCTTGTTGGGCAGCTTATCTGGACACCCTCTGT
TCTGATCTGGGCCTTGACTTAGTCATGAGATGTCACAGTGTCCTCTTATCAGGATTACCCCATCCCCCATTCTGGCCAT
GAGAGCTCTTTCAACTTCAATAGACAACAATACAGGCTAAAGGCCACAGGAATGAGCTCAATATATCATCACTATTGTG
AACATTACTGACATCTTTTTGGAAGAGCGTCCAAATCAAAAGGTGGGGGCAGTAATTGTGAACAACCTAGTAGAAAGGA
TCTTACTTTCCTTTCAGGATAAGGGGAAAAATAGAAAGAAAACAAGGCCACCCACATACATTCTATTCTTCTCAAAAAT
CCAGCCCTGCCAGGAGTAGCTATGAGTTTTGGTTCATTGTCATGTGACCATCACTCTGCAGCCCCTGGTCTGTGTCTGT
CTCATTATTTGACACAGTCTTGATTTAAGAAAAAGGAGCTGCCAGGATCCAACCTGTCAATTACACTGTCTCCACCCTT
TACCCCAAGTTCCCCTGGCTTTCCCTGTGTATCTGTGACACACCCAGGGGTATGTAGGAAGAGTGGATAAAGTCCCAGG
CCTACTACCTTGCTCCCACATACTTCCTGAGTCTGAGAACCCTAAATTCAGCTGGGGGGAGGAGCAAGCCCATCAGAAC
TGGTTGGTGTTCAAATAGCTGCTGTCGCTGGGTAGTGTGCAGAGCCCCTAGGAGGTCTTCTGGGTGTTGGAGGAACATT
CAGCTCTCAGATGGATGTCCGGTTCACTCAGGTGGTTTTAATGATGTTTTTCAATCCTTCCATCTTCCTATGTTACATC
CCGTCTGCCCTACATCTGGATGTTTTCTTGTAGAGGCAGAGCAATGCTCAGATGTCAAAGTCTATGACCTCTAATCTGT
TTGTACAAAAGGTCAGGCTGAAATGGGCTTTTCCTGGCAGTTCCAAGAGTGGATTAGCAGCCTGTGTTTGCCGGTCTTA
TGGCTGGAACGACCTGGGTAATCCATCTTTCATCCTTCTGTCAGTGTGGATGGCTCCCAGGAAGGGAGATGATGGAGGT
GCTGGCCTTGGTCCCCAAATGTCTGAGAACTCTCTAGATGTCCTCCACACCTATGGCTGTCTCAGCAACCCACGCTAGA
AATGACAAAAGCAGCCCACAAAATGTTTGGACCTGCCCGTATTTGTTGTGGACGCCATAGCACCCAAGTTCATTGGAAT
CTATTGCCAGGGACTTTTTCTCCCCAAGTTGCCTCAATAGACAAGGTGGGGGGTGGGCAGAGAAGCAAGCAGGAATGCA
TGTGTCAAAGATGTCGAGGGAGTTTCCTGGCTGTCGCCACACACTGACTCCTTGTTTCTTTAACAGCTTGGTCTAGTTG
GCATTCAGAACCCTATCCTGGAAATCCTAAAATTCTGACTAATTTTGTAAAAAGTGAGAATGTGTTGGCTAGATGAATC
TTACAGAGTCAGAAAGATGTACTCTTTCTGAATAAAGAGTACCAGTCATGGTCCAGAGGACCAAACAGTGGCTCTGACC
ACAGCCGAGGTCTCACTTGAGGAAGAAGCTGGCTGGCGAGACAGGGGGTCACGGGCTCATCTGGGTAGGTGCTTTGCTC
TCTTTACTTCAAAGTAGCTCCACGCTGGCTGACCAAATGCTTCTCTCTCTGGGACTGTCTAGTCAGAGACTATCTTAGT
TACTTTTCTGTTGCTGTGAAAACCACCATACCCAAGGCAACTTATGGACTCATGGTTTCAGGGAGCTGGAGTTTATGGT
GGCTGAGCAAGGTGCTGGGAACACCTGTGAGCTCACATCTGGATCTGAAAGCAGGAGGCAGAATAAACACATAGGCAAT
GACACAAGAGTTTGGAAACCTCAAAGACTACCCCCAGTGACACAACCCTTCCAACAAGGCCATACCTCCTAATCCTTCC
CAAACAGTTCTACTAATTGGGGACCAAATACCCAAAACATATGAGCCTATGGGGGCCATTCTCGTTCAAACCACCACAG
GGACAAACAGAATAGAAAACAGAGGAGGTGGGGTGTAGATGGAGGGATGAAAGAGATGCAGAAGTACCAGTAAGTTATG
TCCCATGAGTGGCAGATCTGTCAAGACTTAAGTCACAGATCCCAGCCTAGGGGAACAATGAGATAGGCATTATACTCCT
ATTGGCAGAATCCTTCCCCTAGGCTCCAATAGGTTAGGCTCATATTCCAGAGCTGAACACATTGCTCGTGAGACTTAGA
GGAACTCACAAAGCTCTCCGAGTCTCCACAGGCTTCCAACATGGTTGATGGCTGTGGCTTGTCTCCTCAGCTGTAAGAC
TGTAAAAGACAATGCCCATAGAGTCTCTGGTGCCTTGTCAGAAACCCAGGCAGAGCAAGTGTTTGACAAGCAAAAGCTA
TCATTATTGTCAACCTGGCCTTAGGATCAACCTCCAACCCCCAAGTCCTTGCTATAGGCACTGCTGATGTTCTGTTTGT
TAATGGATCCATTTGTCCAGCCACCCATCCATCTGTCTATCCAAACTTCCTTCTTTTCAAATAAACAGCTTTGATGTGA
AGTAACTTGCATGTCTGTGTTTCTTGCATCTTGGAGGAGGCTGTTCAGAATGGAAATAAAAAGAACCTTCTAGGTGCAC
TGAACCCAGGTTACCACACCACTTAATCCATGTAGCCCTCAGAGGACCTGGGCTTCAAAATGTCTTCAGTATAAACAGG
ATAGACTATAAATAATGTTGAGAGTGATGCCCTGAAGGCTAGGGTAAGGGACATTTTAAAACAAACTTTGGCAGGGTGC
ATCTAGGGTCTTGGGGCTTTCTGAAGGAATTCTAGAAGCATACTCAGTGACTTAGTGTTAGTTCAGAGACCATCTACTT
GGTGTGTGTGTGTGTGTGTGTGTGTGTGTGTGTGTGTGTGGTGGGTGTGTGAGGGACAGGAATGGTGGGTATATAATAT
GGATATCCTATGTCCATGGATATCTATGTTTGAGGCTAGGATGGATGAGTTCATGGGTGTCTTCTGGAGAGGGGTGTTA
TGCAGTTTTATATCTGGGGAGGGATATGTGTGCAGGTAGGGAGGTGTGGGTGAGGGCATCTCCTGTGAGGATGGGCACA
CTCACATGTGACCATGGGGAGTGTTCCCAGTGTGTGTGTGTGTGTGTGTGTGTGTGTGTACTGCAATCTCTGTGAGTGC
CTGGTGAGGGGACAGCTCTGCTTTACCCTCAGGAGCTCCAGCCACCAGGTAAAATGGTGCCGGCCAGGCCACATGGTCA
CTGGCCATAGAGCTACACTGACAGACTTTACAATGGCCGATTGCACCCAGGAACCAAACTCATTCAGGAACCCGTGCCA
AAGTCCAAAAAGGGGGGAGTTCCAATGACTTTCCTGGGATTTCATCTCAAGGGGCACAAAGTGACCTTTGACTCTTACA
CCTGGCCAAGTTCCTGGTCCCTTCCCTCAGTGAGGGAAGAGAGCCCAGGTTCCTGGTTCCGCCCCACAGCCAGGGAGGG
GAAGAGGTTTCACTTTTCTTCTTGTCATTTTCGCTTTCAGACTCTCTTGCTGAAACGCTGAGGAAGAGAAGCCCAGGGC
CACCGTGAGCAACGCTGGGTGGAGATGTCCCTGCCTCAGGAACTGGCCTGGTGATCAGCCTGATCCAGCTGACCTGGTA
AGACAGGCTATGACCTCACTGAGCATTCTACACTAGCTCTCTGTGCACTTAAGAAAGCAATTTCCAAGAAGAGGTGTAA
CATGGTATGGACATTATGGCCCAGGCCAGGTGGCTGTCAGACCTAGCACTGAGAAAGGACATTAGCTGCTGGTTGGCCC
AACCTCAGGTAAAGGAAGATGCTAGTGTCCAACCTCACATGTTAACATGTGAGTGTCAGGGATTGCGGCCCCCAAGAAA
ACCTGGCTCCCAGAGATCCCCATCCTGACCTTCAGTAGTCATGTCGTAGAGCCTGTGACTGTGTTCTCATGCCTGGCCA
GGTGCTAAGAAGTTGCTGGCCACTCTCAGAGCATCTTATCATACAGGTCACATCTACTCTCTGGTAAATCCACCTTGTG
GCATAGGCTATGAAAAACAGAACCAGCCTCCCCAGCTGCCCACAGCTAGTCTCACTGGAGTCTGAGCCTGGCCCTACAC
TCTGTCTTCCCAGGGTCTGCATCAGATCACATCAGCACACACAGACATAGACACAGATACACAGATGCACAGAGGCACA
AACACAGGCACAGACACAGGCATAGACACAAACACACACACACACACACAGGCACAGGCACACAGACACACTTAGACAC
ACACACACACACATACCATAGATATTTCATACACATAAGCACCACACACATACACACACACACTCTCATATACACATAC
ACAGGCACACACACACACACACACTCTCATATACACATACACAGGCACACACACACACACACACCATAGGTATTTCATA
TCCTAAGATGTCTAGTCTACTCTCGAGCTGCTACCCATCCCTCACGGTCCACCCAAGCTGTTCTTCTCTCTACCCTCTG
CTTGTCCTCCTACCCCCAACCCTCCTTTAAAGGCACCCTTTGTTAACAGGCTGTACCAACAAAACATGTCCCGGAGGAC
TCTTGCCATCCCTCCAGCTGCATCTCAGAGAAAATAAGAATAGGCTTAACTCAGGAGAAATGAAATTCCTGTAAGATCC
GCATCTCCTCCCATCAGGCACTTGGCATTGCAACCTCAAAGGCAGAAATCATCACAAATTTTACGTATAGCATCTAGTT
CACCAACTTCAACCTTCAATCTGTCCATTCAAATTGCCATTAATCTTTCTACAGAAATACTCCGTATGCCCGAGACAAA
ATATCAGAACCATGACATCTCCCCTCTGGAATGAATTAAATTAAACTGAGGTTCCCTTAGAGGTCACTTGAGAATAGCT
TTCCTGTCTCCTGCTGATCAAAGAAGCTTGACTGGAGTAGATGTTTATCAGTAGCATCTTGGAGATGGCTCAAACAGCC
TTTTGTCACTTAGTAAAGGGCCTTCGGGTTTAGTATTTTATTGTCAGGACATTTTGAATTTTAGACCAGCTTTTAGTAT
AAGCATGAACCCCCCAAAATGGGATAACTTTCATGGTCTGCTATACACAACACAGAACATATGGTTTATCTGAAATTCA
AATTGACCTGGCATCAGGTGTCTTATCCAGTGACCTTAAGCTCCTACATCCAATGGCTGTGCTATCAGGCATCAGAGGC
TCAGCAAGGGAATCTGTTGCCATAGCAACCACTAAGCTTGTCAATGATTTTCACTGAGGACAGAGCCAGGTTCTTCCTA
GCACTCACACCCTCTCCAGGGTTCTAGAAAGTAAGCTTAGCCAATACCCTGGATGCCTGGTCCTGAGGTCACTCTGGTG
TCTTTATGTAGCAACTGCAAGCCTGGTAATGCCATAAGCTCACACTTCCAAGTTCCTTCTGTTTTCCTTTCTCTTCTTC
CTGTGAACATGTTTTCCAGAAAGAAGGCAAGCTCCATCAAGAGAGTGGTGAAATCAACCATCCAGCGACACTGTGTCTG
AGCAAGGGCGACCCCTGTGAACACAGCGGGTATAGCTTCCCCACAAGGCCAGAGTTGGGCTTTGGCTCAGCACCATTCA
GGATAACACTCATCTGTAGGTTAAATTACAAAGATTACTCCCTACATCATCTTCAGTGATGCTTCCAGTCTCACTAAGG
GCATCAATGAGGATAGGCAGCCAGATTAGTACTCATTGAAAGTGAGGTATTTGCGAGTTTAGATTGATGTTGCTCATCC
ATCCTGTAAAACATGACATTTTGGGTTGATTTGTTTACTCGTGTAATTGTGAAAGGGCTTTGGCACGTATGCAGCTTCC
AAATTCCACAGCTGTGGCAGGGTCTGCTTTGCTTCCCTGCACACACAGGATTTTGGAGACATGCTGACAGCTGGGGTGG
ACCTGTGAGGGGCTTGGGGGGCTGACTGGCACAGAGCATTGCTGAGAGCTGTCCTTTCCACAGCTTTTAGAACCTCGCC
AGACATATATCCTTCAACAGCAGGCTTTGTTGAAACCACAGGCTGGTTAATGCAGAAACCTGCTGGGAGCTTTCTATTC
CTTCTCGTCGAGGTTTTCCTTGAACTAGGTCCACTGTTGAGAGATCTTGCGTCTTTAACTTGGGCTGGCAAAGGGTCTG
GATTGGGAAGGCAGTGGACAGACCCTACCTTGGGAGATTTGGAGAATGCACACCATGTTCACCGTCCTCAGTGTCTCTG
AGGCACTTGGGAATCTTATTGTAGATAGGCAACTTGGGCTGCAACTTTGGATCACACAGTCCAACTTCCGTCCCTGCTA
GCTGGACTTGAACAATAGTAGTCGTTGGCCACAGCTATATATGGGAAATCTTCTATGCCCAAAGGCCGTGGAAGTAATA
TCTCTCAAGGAAAGCCACTCGTTCCATTGTACCGCTTGATGTAAGTTTAGTCTATAGAGGCTGGTGTTTCTGTAGAGGA
TGATGAATTAAAGCAAGCTAGAAAGAAAGGTTTGGTCCCCACTCCCACATACCCATGCCTTACATCATGACACACTGCA
GAGTGAACTGCAGACCACGCTGCTCTGCCGTATCTCTTAGCTATTTTAAGGAACTTGGACGCATCTCCTAGTCTATGGG
ATGTGGGGGCAATCTGGCTGCCATATCTGTCACCCCATTGATTGACAGGGTGGGTTTGACTGACCTGAGTGGCTAGGCG
GGGGTTGCTTTCTTCTCTCACTGTTCCACATATGTCTATCCTGAAGCTATGTGCTTGGTTGAAGAAGATGACCTTCTCT
GAAGAGAGGAAGATTGGTCTCTGGTCAAGGGTACCAGTCAAGGGTATACAAGTAGCTGCATTCCCCTGCTAGAACTTCC
AAACAACAAGGTCAACCAAGCATGAAAAGAGACACAGCTCTGAGTGGCTCTTCCTCATCATTGCCACTAGGCCAGGAGC
TTGATTGCTATGTACTGATGGCCAAATCAACCCTTGAAAAACATATGCATCTCTTTTTATTGGCTCCTTCAATCGTTCA
TATCTCTGAAGCCCGGGGAATAAGGAGTACCACTTTTTGACTGAGGAATTCACAACGACAGGCGGAGATGCTCACAAGC
ATCCCCTTGCAGCTTCCTGAGCCTAAGTGTAAAGAGAAGCATCTCACTGGGGGCTCAGCAGAGACATCAGTGGGAAAGA
TCCGTTTCATTCCAGATAGTCCTGAGGCCCACAGTGAAGGAATTTTAAGTTATGTATTGCTGAGTGACACTGTCTACTG
ACTCCAAGTGACCCATCCTTTGTTCCCTGGCAAGCCAGACTGCAGGATAAAAGAGAATCTGTTTTATGTCCATTTTGGT
CCCTTATGAAAGGGAAGGGTGAAAAGAAGAAAAAAAAAAATCCTACTGAAATGAGAGCAGCCAGGATCACCTTGGATCT
GCTGGAGTGGACAGACTGTCCAGGAATGAGCACGCTCATTTACTTGCCCTGAAATTATTTATTGACCACCTTCAGCCAG
CTAGGTGATGATGACCCAGGCCCTGCTGCCTCGACATTAAAGTATGGCTGTTTTGAATAATTAAATCAGATCTGGATCT
TTGGGAGAAAGCAAAGACCATTTTATTCTAGCATTCACCAAAGAAAATAGCAAATACTTTTAGGCTTCCTCCAGGGAAA
CGTAATCCCTGCCCCCGCACTTGGTAAAATGTGAGTCCCTGTACCTTCCTTCAGCCCTCTGGGTATCAGCTGCCTTCTT
CCTGTGCAGATAGGATCAGCCCTGGGCTGTGGAAAACAGGGCAGGCATTTGGCACGCTCTCGAGGTAAAGCCATGTGCA
GACAAAGGCAGAGAAAGGAGCCAGTGACTCGGAGGCTGTGCCCCTAGGGAGCTTTGCTGACCTCTGTGCTGAGGCTGGC
CATGAGTTACTTACATCAGCAAATGCCTTTACCTCTTTCCTCTCTGGGGCTGGTATCTTTCTAGGCCTCAGCACAGTAT
CTGGGGACGGGGCAGAGGGGACTGAGGGGTGCTAGAGAAAAATAAGAACCTAAGCTGGGTGCAATGTACAACACTGTGG
GCCTCTGGAAATGTGGCTTTGTGTCCTATTAAAACACACAAGGATGAGGCCTAGAAAAAAATGCAACATGAGGAACCAG
CAAGATGGCTCAGTGGGTAAGGGCATCTGCTGCCAGCTCTGACAACCTGCATACCATCCCTGGACCTACTTGGTAAAAA
GGGAGAAGCAACAGGTTACACACACACACACACACACACACACACACACACACACACACACAGAGAGAGAGAGAGAGAG
AGAGAGAGAGAGAGAGAGGCATACACAGACATACACAGACATACTCAGGCATACACAGATTCAGGCATACATACACAAA
CACAGAGACACACACACAAACACAGACATATATACAAACACACACACACAAACACAGACCCACTCACAGGCACACACAA
AAACACAGACACATGCATAGACACATGCAGAGGCATACACAGACACCTGTACAGGCACACACACACACACGAACACACA
CACAAACACAAACACATACACAGTCACAGACACACATACATGCATACACCCCCCCCACACACACACACACTTTTTAAAA
TGCAATGTGTAAGGTCTCTGGGGTAGGGACAGTCACCACCACTCATGCCTTGCACATGACTCATGCCATTCTCTGCTCT
CGAGTCACAGCAGAGGTGAGCAAGAGTCAGCTGGGAAATGAGGCAGAGGTGAATCTAGTCACTGAAACAGATCAGTAAC
AGGGGACTCGCCTTCGTCAGTTAACAGAGAGGGTGAACTGCCCCTCCCACCTAGTCAGCTCCTAGTCCTCTTGTGTGGG
CCCTGTAGTACTCATTGACAGTACCCAAAACAGCACGATTTCCTCTAAAATCAGGATGCTGCATTGTGCTTGTAGGACC
TGCAGCAACCACAGCAACATCTGCTAACTTATCGGAGCTGCATTGACTCCACCTCAGACCTCCTGAGTTAGGGGCTCCA
GGCAGGGCCCAGAGACCTATGTCTGGATAATCTCCAGGAGCTTGTGGTACACACTCAAACATGGAAGCCACTGCTCCCC
ACATGCCACCTCACTCTGAGGGTTTTACAGTCACCCGGGCCAGGCCACGCAAGCACTGCTGCAGACTAGATCTCAGGTG
CAGCCAGGGCTGACAGAGTGATGCTTGCGGGGCCTGGCTGCTACTCTCTAGACTTTCTTCTCGTAATTTTCATTGCAGG
GCTACAGGGTGTGGATGGCGGTGGGGGAGAGATGACCGCAGCCTGGGTATACTAGAGATCTAAGGGGCTATCTTCTCTA
GAATTCATGACTCCTCTCCTCAGGATCATGGACAAGGAGATCTCGGAACGCTGGACCCATGCCAGAGAAGGGGAACAAA
CTGGGTTCTCATCATAAATCAATGTTAGGCCTGCTGAGGGCAACAGACTTCATGTTACCTACAAGGCATTGCCTTTGGA
ATGGGCTGCCCCTAATTGATTTTAGGTCATCTGACACTTAAAAAGAGAGAAGCGTTTGGGCTAGGGTGGGGTGGACAGG
TGTCTCCTCTCTTTCATCATCAAGAGTGTGATGCCTCCTGCCTTGGAGCGATGCTTCCTTACCGAGGAAGAAGCACCAA
GGCTCATGGAGAAGCCCATGCTCTATGTTCACATGGAGCAGCGAAGCGTGGACTTGCGCTTATCCTCGGTAGGACTTCC
AGGGACTGAGTTTGAAGAAGCCAGAAAAGGGGGTGGAATTAGAGGCAGCATGGAGTCCAAAGGTGGGGGCGGGGTCAGA
ATGGGCCACCTTCTCTACATCTGCAATGCTCCTGAGGAATGTCACTGTCTGGGAATGTCACTTGTACACTTGCCACCTA
TTCAGTTCCAGCATCATCACTGGCCCAGAAGCCCCTTTATCTGCAGGGGTTACCTCCAGGACCCCTTATAGACTGATCC
TTGAAATGGCAGACAGTTTCTAGCTCTACAAATGTACTATACATATACTTACATAGCCACGATGAAGTTTAATTCCTAA
ATCAGGCACAGGTGACTCACCACGGTGACAGTAGAATGGAGCCGTTGTTACAACAGGTTGTAATGAAAGTCACAGGACT
AGCCTCGGTCAAGAGATCCCTGGTCAGTTCATTATTATTTCTATTTCTGTGACAAAATGCCCGACGGAAGCAGCTTAAA
GTTATGTGAGTGTTGTCTCTCCCTCCCTGCTGGTGAGATTGTAGAATGGAATGTCTTCCTAAAGAGATGGGGTAAAGGG
GGCGAGGCAGGAGCTGTGCTAAAGTCCTGAGGCACTATTAACCTTAAGTAATCTTGAGTAGACGTCATTGAGCCAGGAT
GGTGCTGCTGAAAAGCAGCCTAAGGAGGAAAGGACTTTGAGGTTTGGTTGGTTGGTTGGCTGGTTATTTGTTTGGTTCA
CTCATATTTCAGGTTTATGGTGTGTGGTGATAGGGGAGTTACCATGGCAGGGAGCACGAGGCAGCGGATGCATTGCATC
CACGGTCAGGAAGCAGCAGTGACTGGTGATGCTTAGCCTGCTTTGACTGTGTACGCAGTCCACGACAGCAGCCCAGAGA
CTGGTACCTTCCTAGGTACCATCTCAATGAATCCATCCTCATACGATTCCTCACAGGAAGCTGAGGTAGTCCAGGTGAC
TCCTCCCATGAGGGCCTGAAGCTTTGCCTCCTGGGAAATTCCACATTCTGTGAAGTCAGCAAGCAATATCAACTGTCAC
AGGGTAGAAAGTCTGGCAGTTCTCTGCGAGTCATTGAGCAACAGGTGGCAGCTGAGCTCGTGGGATATGAAATGAGAAA
GGATCTGAGATGTATCAGACATGTTTCATGCACAGTTAACATGACAATATATTGTGTTGTTGTAAAGTTTATGAGAAGT
AGAAATAGACTATTATGACACTATAGCCCTTTTTAGGCTATAAGTAGAAACTGTGGTGTATCCTGCATACCGCCTATGA
GATGATGGTCCCATGTCCTCAGCAGGGTACGTGCGTTTCTCTTTGTCTGTTCTGAGCTGCAAGGATGGGCTGGTATCTC
AGAGTTCCTCCTGTGCAAACAAGAGTCATGATATGTACATAACTGGTGCTCAGCAAATATTTGTGCAAGGAACAAATAA
ACATGTCACCCAGGAATGTTCTAGAAGGGAGGCAATGTCAGTGTGGACAGCGGCTTGGACTTTGGAATAGGCCAGATTG
CAGCTTGAGTTCCAACCCTGCCTCCAATCAGTCCAGTGAAGCAGATCCCATAGTCAGCCTCTTCCAGCCTCAGTCTTCA
CTCCTACATGAAGGACACGAGGCCCACGGCTTCTCGCCAGCTTGGTTTTAAAGGCAATATACAGCACAGTAGCTATGAA
GGCAGGATTAGGTAACATGACCCGTGCTCACCGGGAGCTAACAGGAGTAGCAGACTCTAACTCACCGTCTAAGGGGTGG
GCTCCCCTAGGATGCAGAGGATAAATGTGAGGCTTGACAGTCACAGGAGCTAGAGCATCAGCTTGGAGGCTACCTGGGT
GTATGGAAGCAACAGAGAGGAGCTGTGACTGCTCCAGAACCACAAAGGAGAGAGAAAGGCAGGACAGGACCTAGAGTGT
CCCCTTGACCCCACCTGAGGTGGGGACCCTGGTGATGCTTTCCATTCCAAAACTGGGTAGAGAAGCTGATGCGTGTTCG
GGAGACTGCTTCTTCCTCCTCCCCCTCCTCTTCCTCCTCACCCATCTCCTCCTCTCCTTCCTCCTCCTCTCCCCATTCT
TTTTATCAAAGCCCATTATGTTTGCTTCATTATACAACAATGCTGCTAAGATTCAAAAGATAAAGAAAAATGAAATTAT
ACAAGAGCAAAAGTCCACTAGATGCAATTATAATCTTTATACCATACTTTACTGTTTCTCCTAGCCTTACATTGCCTGT
CATAGCTCACGTGTGGTAGGTGTACATGTGTATTCGTGTGCACATGGATGTGTGAAGGCCAGAGCCCAATGCTGGGTGT
CTCCTTCAATCACTCTCCACTTTAGTTTCTGAGATAGGCTCTCATGCTGAAACTGAAGCTCACCAATTCAGCTAGGCTG
GCGAGCCCCAGATCTGCCTCTCTCTTTCTCACCAGTGCTAGGACTACAGACACACATACTTCTGCGTCCTCCTGGAACA
GCATTTTGAAATCACAAATGGCACATATTATATATGAAATATATAATTTTAGAAATTTAAATCGCATTATTAAAAATAT
CTAATAAAAATTGTGTGTGGGTGTGTGGGTGTGTATGTGTGTTGGTGTGTATGTGTGTAGTTATGAGGATAACTTGGGG
AAGTTCTTCTCTCCTTCTATCATGTGGCTCCCCAGGATTGGACTCGGGTCCTCAAGCTTCATGGCTAACAGTGAGCCAT
CTTGCCCACCCAAATTGGCCTTTTTATTTGACATCATGATATAGATGTTTTCTTTGTTATTATAACTTTTTTTGTGATA
ACTTTTAGTCCTGCAGATAGCCTATGGAGCTGTGTTTCTGAACACTTTGGATCCCCTCTTCTCCAGGGGCTGGGAAGTC
TTGCCTCTGCCCTGGTAACCTCTCAGCCATGAGTGCTTGAGGCCAGCAGGGTCTAGTGTGTCTGTAGTATGTGAGACCA
GAGAGACAATGGCTGTACTCAGAAGACCTGAGAGCCAGGTCACATTGACCTCTCCATGGGACAGGCCAGCCCTTGGCTT
CCAGTCTTCTTTGAAATGGGATCAACAACCTGCCTCACAGAGTAAGTGCTAGGCAAGTGAGCTATCAGCTCAGCCCAAA
TCTTGGTTTATTGGATTTACCAGAACCTTCAGAATCCAGCAGCAGGAAATGCAGCCCAGCAAAACTTAACCATAAAGAG
AATTTATTAGCTCACAGAATTGCAAAGTCCAGATACAGTGACCCAGGAGGGACACAGAGCCTGGAGGATATGGCTGTGG
GTCTCAGTCATTGTGGTCCCACCTGCCTACCTTTCTTGTCACCCCTTTCTAGGGAACTTTCTCTCCAGTACTCTCCAGA
CCAACTTCTGTATTAGTCAGGGTTCTCTAGAGTCACAAAACTTATGGGTAGCCTCCATCTAGTAAAGGAATTTGTTGAT
GACTTACAGTCTGTAGTCCAACTCCCAACAATGGTCAGCAGCAGCTGTGAATGGAAGTCCAAGGATCTAGCAGTTGCTC
AGTCCCACAAGGCAAGCAGGAGGAGGAGGAGGAGGAGGAGGAGGAGGAGAAGAAGAAGAAGAAGAAGAAGAAGAAGAAG
AAGAAGAAGAAGAAGAAGAAGAAGAAGAAGAAGAAGAAGAAGAAGAAGAAGAAGAAGAAAAGAGAGAGAGGGAGAGGGA
GAGGGAAAGGGAGAGGGAGAGAGAGAGGGAGAGGGAGAGAACTGAGGACAGCACTCCCATTCCTGGCATCCCCAAGAGA
AACATTGGCCTGTCTGCTTCATTAGAGGTCCCGATGACTAGGCCTGGCCTATGTCCTCCTGCCTCAATTGTCATCCATT
CACTTATACCTCCTAACCTGTGAGTGGGTGAGGGTGGTGGTCCCAAGCAGGGTTAGGAGGTGCTTTCATCAAAAGAGAG
GAAGACTTGGGGAGGGGCAAATAGTAGCCATGCCCTTCACAGACTACCAGTTCCACTGTGGCTAGAAGGAAGTTCTCAG
AGCAGGGAGCCCAGCATCCCCTGTGGGTTTGACAGTCACAATCCTTTGTGATGTGCTCAGAATCTGTCTTGGCCACTCT
CTTCCTCTGCCAGCTCTACCCTAACACAGTAAGCATTTTCTCTCGAATGTCCAATGCAAAGAACTGCACCCTCAGTGCC
TGTGACCATGAACTGGGAGGAGGGGCCTTCAGTGACCACATCAGGTGACTCACTCCACCTTCACAGATATGGTCCAAGG
TGTATTACAGTCCCAGACCCAGCTGAAGTGTCCTGAAACAGGAGGAGGTTGGAACTCTGAGATGGGGCTGGAAATCCCT
GTCCAGTCTCAGCGCCCAGGACCTATCTGTGTTCAGCAGGGAACAAAGATGTGCAGGAAGGTACGTCTCCCATCACCTT
GTGTGCTTGAGAGGCCAGGAGAGGCAGGTGCTCATTGGATCCACCCAGCTGAGTGTTCTGCAATGGCCTCCTGCTTTCT
GCTCTTGTGTGCACTTGGTTGGTCCCTGTCCTGAAGTCTCACAAGTCACCTGGTAGGAAGTCGACCCAAGCAAGGCAGT
GAGGATCTAATAATGGCAAAGCAGGGCACCGGTCCCTTCAGAAGTGAGAAAGTCAGGGCCCCACACTAAGGGGGTAGGA
CCGTGGCAAAGATGCTGAAGGAGGGGAAGGTGGGATGGGTAGCCCAAGGCCCTTCCAGGAATGGAGGACACCACAGATA
AAGGGAAAAGAGATTGCAGGCACTGAGGAAAGACTGAGGAACAAAGGTCTGAGAAGAGAGGGGCCCACCTAGATCCAGA
GCCAGGGGACAGGGCACAGAAAGGACACCTGCTGCAGAGAGGTCAGGAGCTGATCCAGGTGTTTGTGGGAAATCTAGGA
AGTAGGCAAGGATCCTCAGGCCTCCTCATGAGGACTTCTAGGGTTGACACAAGGACAGGAGGGGAAGGTAGGCATTCTG
AGAGGACTGAGGAAGCCTGTTGTCAGGAAGAGCTCTCTCCTTTCTGTGTGTCTTTTGTGTGTCTGTTTCTGTCTGTCTG
TCTCTCTGTTTTCTGTCTGTCTGCCTGCCTGTCTGTCTGTCTGTCTCTGTCTCTTTTGCATGCATGTGTGTGCATGTCT
GTCTGTCTGTCTACATACTGCAGGTAAGACCCACCCTAGCATGGCTGGAGGAAGAATGGAATTTGAGGCTGGTGCAGAC
TCCTAAGCTCGCCACATGAACTTAGGGAGAAATTATGCCAGCTGTGGAGAACACCTGGGCCTGGAACTACAGCCATCAT
GGGTTTGGAAGGAGGCTATGCCAGCCGTGGAGAGGACTTGGGTCTAGTCGAGGCAAAAATCAACAAACAAAACAAGTTG
GGTGCATCCAGGGAGGGACCAAGAGGACAGCACCTAGAAGACAGCCACAGCTCCAATAATGGTGACCTGAGAGTCCAGC
CACAGGAGGGAGTTAAGCGACTTGCTTGAGAATATGGAGGATAGAGCCAAGAAGGTAGCCCCGCTTTGGATCACAAAGC
CCAGCACTTAGGGTACGTAGATCCAGGAGGGCAGAATCCTTCTTCAAAACTGGATCAAGGCAGCTTCACTTACCGCACT
CTCGGTCAGCTTCAAGATGAATGTGTGACCTTGAGCTGCAGAGCTTCACAAGGAATGTTGGGAACAAAGAAGACCTGAG
GACTCCCTGGGAGGTCTTCCGCAGCTGGCTTAGTCAGGTTACGCATCCCACTACACCCCACCCTGGAATGTTGGCCTCG
CACTGTGGAGGCCAGAAAGATGTTGCCACTAGGAAGAAGGGATGAACATGCAACAGGAAAGGTGATGTTAAGTGAGAAA
AAGATCCCCAGGTGAGTTCATGCCCACTGGACTTTGCCAGTTACTGCTGGTCCTCGGTCCCCAGTCAAAGGGCTTGAGA
GGGGAAAAAAAAATCACTATACAATAAGCACATTAAATAATTTTCATCATTCTAACCCAGCAGGTGGTTGAATAACTCC
TGTTTGCTGGCACAGCCATTCATGAGACCAGGAAGAAAAGCCCCTATCATGTATAGCAATGCACACAGTAAAGCCCCTA
TCATGTATAACAATGCACACAGTTAAGATAACAGCTGAAAGCTGCTGTTGCTGGCTTTAGTTTGGTTTCCAGCACCCAT
GTCTGATGGCTCACGACTGCCTATAACTACAGCCTGGGGAAATCTGTATTGGGAAAGAATTAGGAGAAGTAGAGAATTC
AGCAAAATCGTGACACAGAGTGATGGGATTTCAAAAAACCACAACCTTCGCAGCAGAGTTAAACCACTGTAAACCGTTT
TTAGAAGACTATCAGGTTCAGATCATCAGCATGTTAGAAACAAGTACCGTGGCAGAGACCAGCCGTAGCTCTCTATTCA
CTCAGCAGCCTTGGGACAGCAAGACCCGTGGGTCTTCAGTCCTCTTCCTGTGGAGGACTGAGAGAGCATAGAAGCTTGC
TGAATGTGTGCCTGCAGAGTGGATCCCTCTACTCACAGGAGTGACAGCCAGCCTGCCTTAGCATGGGCTGGGGCTATGA
CCTGGCCATGAATGGGCAGTGTGCAGATGGGGCAGGCTAGACCCCCTTCTCCAAGAAAGAGTGAGATCACTGCTAAGGG
AGTGATTTCGGGAGATGTGTGGGAGAGGCCTGTGGAAGTTTCTCCTTTCTTTACCAAATGACCCGCTCTGGGTAAGACC
AGGAGCTAGCACAACAGAGCCAGATCCTGAAAAAGTCATCGTGAATGACCACTGACATTATCCTAGGGGTGTGTGCCAG
GCCCTGGTACATCCCAGAGAAAACGGAGAGCAGCAGGGGGCATGAAACTCTGGGGCTGTGATAGGCCCCACTGGTCACA
CTGCACTTTCTTGGGGGAGGGGAGGCTGGGGTGTATCTGAGAGCTGAGAGGGGGAGACAATGACAGGTAGGAGGCCTCC
AAGGACACCCATGTTGGAGGACCAGAATCTCAGGACAGCAAGAGCAAAAGAGTGGTCTTGTGGCTGGACCATCAGGGCC
CACCAATGGCTATAGCTTCCCTTGGACTTTTGTTTAGAGAACTTACCAGGCTGGTTGTGATCGTGGCAGCAGGGTTTAC
ATTTTTTCAGTAGAAGGGAAGGGCTTTCTAAGGGGCAAAGATGGGAGATGCATCCCTCCATGGGCTCAGGACTTGGCTA
GCCTATAGCTGTGACCCTCAGACTGTGTGAAGAGCCCCCAAGTGCTCAGACTCTGTACCTGGCTTCCATGTTTCTATTT
GGGCTCTGCCACTTAGAAGCTCTGTGACTGACCAAATTATTGTACCTCTCTGTGCCTTAGTTTTTCTAAAAAAAAAAAA
AAAAAAAAAAAAAAAAAGTTCATTATGTGTATCTACATGTGTCTGTGTAAGAGTATGAGCATGGGAGTGCAGTTGCTCA
TGGAGGCTAGAAGAGGGTGCCAGATCCCCCACGCTGTACTTACAGGCAGTTGTGAGCCATCTGACATGGGTGCTGGAGA
CTAAACTCCAGTCCTCTGCAAAAGCTGCACTTAGGCTTAATCACTGAACCATCTCTCTAGTCCTCTTGCCCCAGTTTCT
GCAAATGTAAACCAGAACTACAAAAAAGCGTTATCTTTCCTTGGATTGTTTAGAATATCCAATGAATTTTCCATTGTTG
GAAGTATGTGTTAAATAAAGATAGAAAACTGTGTAGGATATCTCATGAGTTGTTGGCCAGGAAGGACCCAATGGTCCTC
AAATCAATTCAGGCTATTGCCATTGCTCTTGATTGCCCACTAGAACTAGCTGATAAAGACCATATTGCTGAAGGTATCA
TATACTTTAGTTGCAGAACATAGAGAAATCAGTCTAAACCTGAACTAGGAGTGTCTTACTGCCAGCTGGTCTCCAGAGG
GAAAATGTTCTAAGTGCAGCCCAGGCACTGGAGAAGGAACACTGTCAATGGTCTTATCTTGCTGTGAACCCTGAGAGTT
ACACCACCAACCAACAGTGCTGTGACTATCATAGAGGTAACCAACCACTTTCTTATTGGTTTGTAGACCTGCTTCACAG
GAGGAATTCAAACCAGGTACTCTAAACCTGGTCAAAATCCCACGGCTAGGGAGGTCATAGGCCCTAGAGGGGTACCTGT
GACTTTTGTTTTGCTAGGTGGACATGGTGTCAAACTGCCCTGTAGATATCTCTGTTTCTACTAAACATCAGTACTGTTC
TCAGCCTTGGTCAGAGAAGCATCTGCACTGATAATAGAGATTCAAATGGCCAAAGTGCTGAGAAGTGACCATTGAGCTC
GAAGCACTAAACGGGACAGCCCGAGGGCAAGAAGGAGTACCATAAAATGCTGTCCGTGGCATCATGGACATGGCTTTTG
GCATCATGAGCTCACAGCTGTAGCGGCTGCCTGCACAAGACTGGGTCCATCAACATTCCGTCATGGACAAGGGAGGGAC
TCACAAGGCCCCACCCCTCCCCGAGGAACCATAGGCAATTAATGGTTGCTGAGGCAGAGAGAGTCCTCAGTGGTCTAGC
CACTGGTAGGTTGTTCTTGCTCAAGTAAATAACTCTCCACCCATGCTGGTGCAAGCAGCCCTAATTAAATGCAGTGGGT
CCCAAAAAAACAAAACAAAACAAAACAAAACAAAACAAAACAAAACAAAACAGCAACACCAGAGAGAAAGGTATGAAAG
TAGGAGGAAGGTTGCTGGGAACACGAAGGGGTTACTATGAGGGGGCTGGAAGAGAGGATATGGGGGGGTGGGGAGATGT
GGCCAAAGTAAGTGTGTGTATGTGTGTGTGCGCACGTGTGCACAGGTCCCTGTAAAAGAACAAAGAAAAAGCAGTTAAG
ATAGTGAAACATATACAAGAAAGACTTGTGCCTTTAGCCATCACTCAATTTGACCCAATTATGACCTGTGACTAAAAAA
ACATCTAGGAACATTGTGCGTTTACACTTGCTCCTTTTTTGAGTTCTCCTGGACCCATAGAATTTGGCCTTCGACCCTA
TCATCCACTGTTAACACCCTTGCTTGGCTCCGCAGTGCAGGACCTGGCCAGCTCAGTAGCTGCCCCTCAGGTCATACCT
TGCTCACAGCATGGACAGTCTTTAGTCTTCCTCTTTGAACATGACCTCTGGTCTCTCCCCACTCCTCTCTCCCCTGGCT
TACCGCCTTCTGCTCTGGGTCTATTTCTTGGGCAGATGTTTCTGCAGTCTTTTCTGGATGTCCTTCTCCCCACACTCTC
ACGGAGAGACTTCATCCTTCCTGGAGGGTTAGCATCACCTCCACCCACCCTTTTCTTGACTTCTGTGTCTATCTGGGAC
ATTGAGTCATCATAGCCGCCCACCAGCTGCCCACTTTAAAAAGAATTTATTTTAATTTTAAAAATTATAATAACACTTT
CCCCTTCTACTTCCTCCAACTCTTCCTCTGTTCCCTCTCAAACTCATGGCCTCTTTTGCTTTGTTGTTTTACACACACA
CACACACACACACACACACACACACACACTATATGTAAATATATAAATGTAAACCCAACTGCTCAGTCCATTTGGTGTT
ACTTATATGTATATGATTTCAGAGCTGACCACTCCGAATTGGGCAACCAAGTAGAAGGCTCCTCCTTGGGGAAGACAAT
TTCTCCCACTCTCCGCATTCCTTAGCTGCCAATAATTCTTTGCCTATGGGCGGGGCCCTGTGAGATTCCCTCTCTCTGT
GTTAGCACGTCTGTTGCTATCATCCTTGTACAGGTCTTGTTTAGACACCCATATTGTTGGGTATCAAAAATGAAGCTTT
CCTTTTGTTTTTAGGAGGCAGTCTCACGGCAGATTTCCCGGTCCTCTGGCTCTCAGATTCAAGTGCTCCGCCACATCTT
CCAGAACTTTCCATGAGCCTTCAGTGCAAGAGTTGTGTTGGAGACCTATCCCCTGGGGATCACTTGCTCTTAGTACTTT
GACCAGTGGTGGTTTTCTGAAATGGTGTCCGTCTGTTGCACAGATAAGTTTATTTGCTGAGAGACGAGAGCTACAATTA
TCTGTGGCCATATGGATAAATATTTAGAATCCAGCTGCCCGTCTTTACTTGTATGTAGTTCATACTGGGTAGAGCCTCA
GGACACCACTGGCTTCCCCCACCCCCACCCAGGGCTGCTAGTCCTTCTGGGTCCCCTCTGCTGGGAATGAGCATGGCAA
ACTCATGGCAAATGCTGTGTTGGAGTCCTCACTGACGTGGGTACCACCAGCATGTGAGTGCTGACTCCCTGCAGTATTC
CCTGAATTTTTCCTCCCTACCTCTTGCGCAGTCCAAGCTTGTATCGCTGTTTCCGGAACTCTTTCCGGATCATGGGAAA
TGCAAATCAGCTTAGAGCCCATGCTACTCAGAAATCTTCAATGTATTGTCCCTGAGCTCTCGAGATAAGGCCTCCGTTC
TTAGCATGCCAGTTGGTTTTCCAGTCTCTCCCCAGCCTCAAGTATTCCTGTTGTTCCTGGTTGTGTACTCCAGACAGAA
TGACACCAGGGAGTAGATGTACCCTCCTTCCCTCTATCCCTCCTCCTCATCCTTATTCTTAGGGGGTCACTTGACAGTT
ACCTCTTCAGATAGTTCCCCAACCTGCTGTCCTGAGTGCATTGGGGCCCCTCTCCTGCATGTTCGTAGCTCCTGTGTGC
AAGTCACTCTGTAACATGGCAGAAGCCTCCTCTCTCCCCACACAGACAGAATGGCTTAGCGACCAGGGCCTGCCACTGT
GCACTGGGACTGGGCTCTTGTGTTTTTAGCACTAAAAAATATTTCCTGAAACCAAGGGTTGGGAAGTCCAGCAGAGAGA
TACTGGTGATGCTCTTTTGAGGATGCGGATAGCTCCACGTGGAGACACTCAGTGTTGATACAACAACCTTTCTCTTCTT
TTCCCTAGCAGAACTTCAATGTTACCCTGGAGGGCACCATTTGTGTGAAGCTCGTTTCTTAGTATCCTTTGTAGGAGGT
TACTCTGTGGTCCAGCCAACAAGATTTAAGGCAAAGTCAGTGTGTGAGAAGTTGCTTCTCGGCTGTGAGCCTGGCCCTT
TCCTGTTTCCTGTCTTCTACTTCTTTCCTGGAATAAAGAGAGGAGGCTGGAGGAGGAGCAGCCATCTTGTGACCATGAG
CTAGCCCTAGTCATGGGAGCCACATAAGGAACCCTGAGGGTGTCAGGTCATGACCCTCTAACTACCAACAGCCAACCTC
TTACAACAGACTGTGTTCCAAGGGAGCTCCTTTTCCCCCAAATCCTGGTGGTTACTCATTCCCTTTGTAGCCCTGCAGG
TTCAACTACGATCCCTCTTTTTTGATTCCCCTCCCCCCATCTCAATCTCTTTGTCTCCCCCCTCCTCCTCCCCCTCCTC
CTTCCCTTCTCTAGGTTTCTCTGTCTTTCTGCTCTTCCTCGGCTTCTCTCATGGCCAGGTCTGGGCTGCTGGCCATGTT
CAGTCTACTGCTTTCTCTCTGTGCTCTGGGCCTTTCCAGATGTCTCTGTCAGTTCTCTCCCTCACATCGACAACAAAAA
CCTTCCCCTTAACCATACCGTAGAGTATCCATGTTCATACACAGACAAATAAGTCTCACTGGAGTCATTGCGGTCAGAC
GCCACTGCATTTAGCTGAACTCTCTTGATAACATTTCTGAGTAAGCATTACCCTTGGAGGTGACACACTGGCCGTGATA
CCAGGCCGAGGGCAAAGTTAAAGGAGCAGGACAGCATCTACACGGGAGAGCTGGATTCTCCCTGGGAGTCTACTAGAAG
GATCTACCTGGGAAGGACACGTGTTGAGGAAGAAACTCTAATCACTGAGGCCCCAGGGTGAAATGCAATTGCTGTACTA
GATAACTTTAAAAGTGCCTGAGGCTAGAGAGATAGCTGTCAGTAGTGGGCCTGCCTGGCAAGCAGGAGGACCTGAGTTC
AATCCCATGTAATCAAAGAGAACAATTATGGCAGCAGGCACTTATAATCACAGGCCTGGGGAGGCAGAGACAGGTGGCT
TGCTGGCCAGCCCCAGGTCAATGCCAGGCCAGTGAGTCTGTTTCAAAAACAAGATGGAAGACATCTGAGAAACACGTGG
GGCTGTCCTCTGGCCTCCACCCTCCTTCATTCTACACAAGAGTGCTTCATGGCTGGGCTTTGGCATCAGGCAACACTGA
GTTCTGGGTCTAGTTCTATTACTTCCTTGCAGTGTGGCTTTGGTCCCTCTAATTGATCCCTCTGGCATCCTTACCTGTA
AAGTCCAGATCATCAAAGCAACCCTCTCTCACTTAGATCTAGCACTGGTGGTACAAGAAATCTACATAGCATCAGGCAC
AATCCACCATGGATGGGGCCTTCTGTGTCCAGTGTACACTGTCACCTCTAACTACCTGTGTCCCACGGTGGTCCAGTGA
GCCAGTCTGTCTGTACTTTTCTCTGCAGGTGAACAAAGCAACGAAGAAGTTGCCCACTGTCCAACAGCCTCTGGATTAT
TGCTGTGTCCAGAGTCCTTGGCCTCGCTTGCTAGTGCGTAGGAAGAAGTCCCCAGACCGACCAGACCAGAGTTGAGAAA
ACATCAGCCTTCAGAAATAACGAAGCAGTGGGCTCCATCCTTCACCCATATTGTTCTCCATATTGACAGCATCTTTTCC
TGGGCTGGGCTGGAAGAGGTGGTTCTGCCCAAGACAATAGAGTAGGAAGCTGGAAGGGCGCCTCCTCCGAACTGCCTCA
AGAGCTTTTGCGGGATCTATTTTGTCTTTTTCTGGAAAAGGCTCACCTTCTTTCCCTAAGGTCCCAGTCCAGGAGAGGA
AAAAATATCTTGCTCACTCCAGCCTCGCAGAGGAGGTCAGGGTTGGGGCAATCCCTGTCAGACCAGCGCTTCCTTTTGT
CCCAACCCCCAGCACTTCAGGCCAGACCCGCTGACACTCGACAGCGCCTGCAGTTTACCTTGGCTCCGGTCCAGTGAGT
TTGAGCCCAGCCCTGGGGCGGCCCCCACCTCTCCACCTTGTTACGCCTTCCCACCCTCCTCTGCAGCGCAAAGAGCAGT
TTAAATATAAATCAGTCCTCAGCACAAACAGAGCTTGGGCAGCGAGTCTCAGAGCCATCACCGGAACCGAGGGCCCAGG
TTTGCGGAGCGAGTGCAGACACGCAGAGCCCTGCTCTGGACAACCCCAAGAGCCACAGAGACTCCTCGCTCCCTTCCAT
CACCCCGTGCCCCTCGAGTCCACTCTAGCTCCCCACCCCTTCCCCAGTGCCGCGCCCACCTTAAATCCCCAGGTTTCTC
CTGGCCTGCAGCCTCCTGCCTCCTCCATACGCGGTCTCCTCTTCCTCGGGGACCCGCCATCTCCCCACACACTACCCCC
AACCCTCAACCCTGGCCAGCAGGACCTGATAGGCAGCCAAGGCTCACCCAGCTCGAGTCCCAGAGCCTCTGGGCTTCCG
CAGCCGCGACTCCAGGACCCAAGACACCTGCCTGGGGGAGGCGACAGCGGCTGGGCGGGGGCTAGCAGCACCTCTGACA
CCCGAGGAAGAGAGGGAGGAGAGAGAGCGCGTTTCATCATCGCTGCCACTTATAAAAACTTCTGGGCGCTCACTCTCTC
GCTCAGTCTCCACGTCTCGCTCCCCATAGCCCGGCTACCGCCGCTGAAGCCTGAAGCACCCAAGTCCCGCAGAACCTGA
CCCCGGCCTGTCGCCACCACCTTGGCCCGGTCAGGGGACGTCGCCTCAGTCACGATGGGGCTCCTGCCCAAGCCCGGAG
CACGCCAGGGCAGCGGCACCTCCTCGGTCCCAGCCAGACGTTGCTCCCGCTCGGTCTTCAACAACATTAAGGTGAGTAG
TGTTGTGAGTAGTGTTCGTGTTCGGGGCAGGAGGGCGGCAGAGTGGGTGACACAACCTAGGACGTCGATGAGGCCAGTC
CGGGAGCTCTGGGTTGCACCCGGCTCCCTTCTCTGTCCCCGGTGCCACATCCGTGGGGCCTGGCCCGGCGCGTGAGCGT
GAGCGTGAGGGGGCGACACCTCTCTCTCTCTCTCTCTCTCTCTCTCTCTCTCTCTCTCTCTCTCTCTCTCTCTGTGTGT
GTGTGTGTGTGTGTGTGTGTGTGTGTGTTGCGCGCATATGCTCATAGGCTCTCGTGGGGAGTAATAATCAGAACAGACG
TAACTTTGTAAAGGGGAACTGTCCTGTTCTTCCATGGAGAAAGAACACCTCCAGGGAATTCTGTTGGGTCTCTGCTGTG
GGTATGCGCTCTTCCTTAATGAGAAGGGATGCAGAGGAGTTTAGCCAGCACCTTGTGGGCAACAGGCTGTGGGCAGCCT
GAGATGCAAGTCAGAGCACAGGCACCTTGTCTCAGTGGATGGTAGAAGCCTGCATTCCTTGCCTGGGTTTTCATCTTTG
CAATAGTTGGGAGTCGCAGATTAGATTGCTACCTGGCTGCATAGCTCCCAGCAGTTTTGAGGATTCCCCAGCTCTCCTG
GTCTCAAGAAAACAGCAGGCACTCGAGGTTTATGGAGAAAGGCCACCAGAGGTGCCTGTGGCCTGAGAGTTATGAGGAG
TTAATAGAAAAAAGAGGAAGCTGTTAGCATGAGAATGCCTTCTTTGGTAAGGGTCAAAAGTAGGGTCCACTTCCCCCAG
GTCTTGAAGGACCTGGAATGGTTGCGTCCTCACAATATTTGGGAGAGAGGTTCCTGAAGAAGGTATCTCCTGGAAGGGG
CTAGACGACCTTGGTCAGACATCCAGGCGCCCTGGGGTCCGGCAAGCATATGGTGCAGAGAGATTATTTCCCACAATTC
CTACCTTAGATGTAATGGGTGCCCAGACCTCACATGGAAGGATTGGAGAAATGTGAACAGGCTATGTGTGTCTAACGGA
AAGAGACTGGGGTTTGGGGGGTGGGGTGGGGCAGAGCGAATGCCACCTGCATGTTCTGCCTCAGGCTGTTACTGGTAGA
ACTGGGGTTCCTCGTGGGGATCCCTCTTTTGTTACTACCACCACCACCACCATGGTTTTAAAGGGCTACCTTGATTTCC
CATTGGAATATTCCTAACACAGAAAATTGGAGGTAATTGAAAACAGTCCTCAGATCTCCTCCCCTCTAGCCAGACCTGG
ATGGGAATTCCATTTATTATTTTGGTTCTACCAATTTAGAGGGACTTGGAGAATTATGGAGATTTTTTTTAAAACTTGC
TAATTAGAAGGAAAGGGTGTGTCTAAAGGATGGGTGGTCTCAAAGGAATGGGATTGTGGCAGCACGAGAAGGAGGTCAC
TCTCATTTTCCCCAAGAATGGAAAGAGAGGTGGCTGTCCCAGGATTCCTGATGGTGAGGGTGACAAGAGTGACTCAGCA
CCTCCAACCCCTAAGAATAAGTGGGTGGCAGCCATTTCGTTGAAAAATATAAAAATAAGATAAAACCCACATTCATATG
CTGAGATTCATGCCTGAGACCTGGACCTAGGCCAGTTGGCCTCAGAAGGGAGAGTCAAGAGCTCTTATCAGTCAAGAGG
TTTTGAGTCTGTTAAAACATTTCCTTGGTTGGAGGATAATAAGCATCAGATGACAGGCAATTTGGAGAGCCCCCACCTT
AGCCAGGTTCGTCCTGGAGACCCTAGGCACACCTGAGTGGAGATCCTAGCTAAGCTTTTTCTTGCTGGTGACTCTATGC
CTGGGCCTTTGCTATTCGAAGGGAGATTTTCTCCTCTTGATAACAGGCTGGGTGATTACCTTATACCTTCCCAGCAAGG
GAAAGGTGACACAAGTGACACAAGTGGGGCAGGTGACAAGGGACATCCAGCCCAATAGCTTGTCACTTTGCTGGCATCT
TTGTCACTGGGATGGACTGTCAAGAGCGGATGTTGCTATAAACCCATGTGCCACATGATCTGAGGGAGGAGTAGAGTCT
GAGAATACAGATCCACGGCCCAGCCCGGCCACACTCACTTCCTGGGTCACTCAGGACAGACACATGGCTCACCTTTCTT
GGCCCTCTGGGGTCCTTTTCTGAAAGAGTGGGTGTCATGAATAGTTCCCACCATGCACCTCAGTAAGAATGCCCTAGGG
TAAGAGTTAAACTGGGCCTCCCCAGGGTGAGCCCCACTTCTGTAAACAAGGCATCGCATTCGATGTTGTCCAGGAGCTC
CGGTTTCAATGTTAGACCAGCTCTGAATGACGCCCTGGTCTCCATCCTTGACACCACACTGTGAGCCTGCACAGGTCAA
GCCAGATAGGAGGGTACTGAGGTGGGAGAGTGAGTCCCACCTCTAACCAAGATGCTATTTGTAATTGATACTTACCAAC
AAAGGCAAACATCCGTTTTCTTCAGTGGAGTGTCCCTGTGAATATCAAACCACACTTCAGAGCAGACCCGTATCCCCGG
GGTAGCTGGCCAAGATAAAACAAAACCCATTTTTGTTTGTTTGTTTGTTTGTTTGTTTGTTGTGGGCTTTTTGTTTAGC
TTGGGCATTTTTAGCTTATTGTTTTGTTTTGTTTTGATTTTTGTGAGATTTATTTATTTATTTATTTATTTATTTATTT
ATTTTAAAGCCTACAGCACCCTCTGGTCTCCCAGTAGTAACCAAGCCTTACCCTACTTAGCTTCCAAGATCAGACGAGA
TTGGGTGCATTCAGGGTGGTATGGTCATAGATGAGTGCTGTTTTGTTTTGTTTTGTTTGAAAGAGAGAGAGAAAGCATA
AAGTTGAGTGGTTGGTGAGGTGGAGAGGACCTGGGAGGAGTAGGGGGAGAAAAAACATGATCAAACTATAATATTGGAC
AAAAGTTGTTAAAAATCTTTAAAAATGATTAAAAGATAGATTTTAAAAACTCAAAATAAAAATTAGAAAAAAACTGGGT
GTGGTGGTCTTGTAATCTTAGCATCCAGGAGATAAAGGCAGGAGAACTAGAAGCTTAAGTTATCACTGGCTATAGAGTA
AGCTGGAGACAGCCTGGGCTACATGAGACCTGTGAAAGAAAGAAAGAAAAAGAGAGAGAGAGAGAGAGAGAGAGAGAGA
GAGAGAGAGAGAAAGGAAGAAAGGAAGGAAGGAAGGAAGAGAGAAAAAAGAAACCATTGTCCCATTAGTTCCATGATGC
AGTTATTAGTTATCAATGACAGTTAATAATATTAATCACAATGTCCATAATAATAACAAATAATAGCTAACAGTAATTG
AGCTCTATGAATGTACAGACCTTGAGGATCTTGCACGCACATCCCATCTGCTTTACAACTCTAGCAGAGGATAACATTA
CCAACCTCACTGGTAAGGCAATCAATTCTTCATGGACCAGGAAGGGAGGCAGGGAGAAGCAGGAACAGCTATGGCACAA
AGTACTGTCCTCATGAGCACTATCGTCTACGCTGGCTCTATAACTGGCGACAACACAGCCATCACTTAACAGTACTTTC
TCCAGGACCAGAAGACATTCAGAAGTGGGTACAGAGGGGACCCAGACATGAATCCCCAATGCTGACCCACTCCTGCCTC
TTCTCTCTTCACAAGGCTCCATGAAAGAGCCTTGCACCTATTTATTATTATTATTATTATTATCATCATTATTATTATT
ATTACATGAAACGGTGCTGGAATTAAAGGTGTGAGGCACCACCATGCAGCCAGGGGAATTTTTACATAGCCTGTGTGTG
TGTGTGTGTGTGTGTGTGTGTGTGTGTGTGTGTGTGTGTGTGTGTGTAACAAGTTGATGTGCAGCTATGTGGAGGCCAG
AGGACAATCTACAGTATCAGCCTTAGGGACAGCTTCCATCTCCTTTGAGAAAGGGTTTTAAATGGCCTGAAGCTCACTG
ACTATGGTCAACTAAGCCATGGATCCCTAGCCATGGATCCCTAGGGCTCCCCTGTCTCTACCTACTGCTAGGGTCAAAC
CCACTCTGTGTCTGGGTAATGTCATTCTTACACAGGTACTAAGGCTAGAACCGGGATCCTCCTGCGCATGCGGCAAGCA
CTTTATAGGTTGGGAAGTGTTTTGCTTCTGGTTTATTTTGTGGTGCTGTTGAATCCCAAGCCATGAGCCATATAAGAGA
GCATTCTACCACTGAGTCACCTCCCAGGCCCTGTGGGGTGAAGATTTATATATCTCCTCCTGCAGGAGACCTCTCTCTC
TCAAGATTTATTTATTTTGTCTATATGAGTACACCATTGCTCTCTTCAGACACACCAGAAGAGGGCATTGGATCCCATT
ACAGATGGTTGTGAGCCACCATGTGGTTGCTGAAAATTGAACTCAGGACCTCTGGAAGAGCAGTCAGTGCTCTTAACCA
TGGAGCCATCTCTCCAGCCCCCAGGAGACATCTCTTGAGGTTTGGTTTGCAAGAAGATTCTTAGGAATCTCAGAACCTG
CTCAAACATCACTGTCAATTAGTCTAGTACAGGCCCAGGACTCTAAATTAAAAGCAGGTTCCCTCGGAGACCCTGGGAG
GAGGTAGGCTCTGCTCCTGACTTCTCCTGGCAGCCTCAGACTAATCTGAAGCTCCCTAAAGTGAAACACCATCGTGGAG
CTAAGTCGAGGTTCTTCTGAGGCCTTTGACCCCCTCAGCCCTAGTGAGGCATCTTGAGTGCCACTTCTTAGTTTCCAAA
ATCCAGGGCAGCATGCCAACAATCTAATCCATCCCCAGGAAAACACTCCCACTGGACTCCCTGAATCAGAGAAGAGCTA
ACGGTGGCTCGCCCACCTCCCTCCAGGGGGCGCATTTCCATAGACTTCAGAACAGTGATCTGTTCTCAGGAGGCTGGCT
TGGCAAGTTTATCAGAAGGCAGGGAAGGAAGGATGAGGACTAATGAGTTCTCTGGTATTAGAATACTGGGATTCCTCTC
TGGGATGATGTTCTAAGCCCTCTATAGCTTTTCATCTCCCAACACAGAACAAATGAGAGCAAGTTAACATTTCTGAGGG
TGCAGAGTCCCTTTCATCTGTCCTGATTGTGACAGGACTTTCTCTTTTTCCAAGCTAGGAGACCCAGCCTGGGCTGTTG
GGATGAGGGCACGCTTCTCCTATCTTCTCTTGTCTCAGCATGTAATTTGAGACAAACGGTGTTTGTAGGGGAAAGTTAG
AGGTCTCTGACCCTCCTCGTTCTCCCCTTCCCACCCTTGAATTGGTTGTAACCACCTTTTTGTCTGTAGAGACACTGGG
TGCACAGTGTCTACTATGTGCCCAATCCCAAGGATGGGGAGGGAGGTACCAAGAAGGCAAGGATAGGGAGACTCAGCGC
TGTGTAGATAGAGGATAGGGGATAGAGGATTGCTCTCTGTTTATTATTTTTAGACTCTCTAGAGATGGTTTTCTCAAGG
AATGCTGTGAAGAAATGAAGAAATGGGGTGTATGAGTTTGCCCTTCTTCATGCTCTACATGGGTCACCATCACTGGGGG
CTCTCTCACAGTGGCCCCATGAGAAAGTCACTTGTGTTGCTTAGTTCCAGGCACAGCTTTATTTGTTTAGAGTCTTTTA
GAAAATGGTTTTCAGTTTGAAACGATAACAGATTCTCAACAAAGCTGGTCTTAGAGGTCCTGTGTATCCTACACCCACT
CACCCTCAGGGTTCCATGCTATATGACAACAGTAATGACGAAGGATATCAAAAAAGCAGGAAATCACCACTGCTATCTC
GTGCACGTACAGTCCGGTCCTGTACTGCTTTATCACAGGTAGATTTGTGCAGACACCACCCATCTGAGGATACAGGTGT
GTTTCACCACCCACCGCAGAGAAGTGCCTTGTGCCTGCTCGTTGAGAGTCTCATCCTCTGACCACTGTCCCTAACCCCC
AGCACTGTCTAGTGTCCATCTCCAGGATTGTATTATTTCAAGGCTGTTACATACAGGGAAGCATGTAACAGGCGACCTT
TTGAAAAACCAGCCGTCTTCATGCAGAATAACGTATTTGCGTTCCAGTCCGTCTAAGCTGTGACACAGATCAGGTGCTG
GTTCCTTTAACGGCTGAGTACTACGCCAAGGTGTGGATGTGCTGTGCTTCGTTTACACATGTTCACTTATGGAAGAACG
TTTGGCTCATTTCCCACTTGGGACTAGTTCAGATAAATCCACAAGAGTGGTGACGTACAGGCTTCTGTGTGGACATAGC
TCGTAATTTCTCTAGGGTGAATGCCGTGGAGTGTTGCTGGGTGGTGTGGTTGTTGAACAATGATACTTTTTTTTTAAAA
AAAGATTTATTTATTATTATAAATAAGTACACTGTAGCTGACTTCAGACACACCAGAAGAGGGTGTCAGGTCTCATTAT
GGGTGGTTGCAGAGATTTGAACTCAGGACCTTCCTTCGGAAGAGCAGTCAGTGCTCTTACCCACTGAGCCACCTCACCA
GCCCTGAACAATGATACTTTTTTAAAAAGGATCTTCCTAACTCTTTCCTGGAAGGGTCGTCTCACCTTACACGGGTGTC
AGCTGCTTAGCAGTTGGTCAGCTTTTGACATTACCACTCTTTTTTTGTAAAAATTCTAATAGACATGCGGCACCACCTC
TTTGTGTCTCGTTCATGTTGTCCTTGTGGCTGGAGAGAGGAAGATTGAGCTTCTTTTCATGTGATCATCGCCCCTTCAT
GTTGTCGTTTTCCTTTAATGTTGTGTAGCTTTAAGAGCTCACTGGGCACTCCAGTTATAAGTCCTTTGTTGGGTTATTT
GGTCTGCAGATTTTGTCTTTGAATCTGTAGGTTGGTTTTTTATTTTTTTTAATCATTTAAAATGGTTTTATAGAGAAAA
TACTTAATTTTGATGAATTCCAATTATTGGTCTGTGTGTGTGTGTGTGTGTGTGTGTGTGTGTGTGTGTGTGTGTGTGT
GTATACACCTGCTCACATGTGAGCACACAGTAGTCAAAGGACCTTAGGTGTTGGTCCTCAACATTCACATTGTTTGAGA
CAGGGTTTCTTGTTTACTGCTATATACAACTCCAGCCTTTGTTTGTTTGTTTGTTTGTTTTTAAGCTCACGGAGGCAGA
TTACCCACTACTCACTGTGTAGCTCAGGTTGTCCTGGAATTCCTGATCCTCTTGATTTCTATCTCTGAATCGCTGGGGT
ACTAAGTATGTACTGCCATGTCTGGCTTACATATATCAGCCTTCAGCAGTTTGGGTCCTCCTCTTGGTGCTACATCTAA
GAAGGCTTGCTTCACTGAATCATAAATCTATTGCCTTTCGAGTTGTTCTGTGTAGAGGTGAGGACCGGGTCAAAGCCTT
GTCTGTTTTTACCTCTAGATGTTCTTGTGCCACGTCTCTTCTCTCCTAACCTGTCTCCGCGCTCTTGTCCAAAGCCAGT
TGGCTGTTCTCCTGTAGGTCTCTTGGTTCTTCGTTTAGCTTCACTGCTACACACCTTCAGGCCCCTCCTTGTATCGCTG
TCTGATTATTGTAGCTACGGAGCGAGGCTCGACGAGGGAGGAGCTTGCTTTCACCTGTAACGGTGCAGTTATCAAGCTT
GCTAACTCACGGGGCTTCCCTTGCCCTCTGGCTTTTGGCATGATTTCAGGGCAGATTTCAGCAGTGGGGATGAGCTCTC
TGTCAGCACCCCTGTCTCCTGCTTGCCTTCTGGTTTCAGCTGTTTGGCATGCTTTTCTCCTTGGACTTTTAGATCTGTG
CCTCTAAATCCTTCTCTATGAGGGTGGGAGATGTCTCAGTCTGTAAGGTATTCACCATGCAAGCATGAGAACTTGAGTT
CAGAACCCCAGGATACATGTAAAAATCTGGGAGCCATGGCCCAGGCCTCTAATTTCAGTGCTGGGAGGGTAGAGATAGG
TTTCCTGAGGCTCCCTGGCCAGCCAGCCCAGCAGAACCCATAAGTTCCAGGTTCAGTAAGAGAATTTGCTTCAAAACTA
GAGACTTGCTGTCAACCTCTGGCCTCCACAAGCACATGCATGTGCACTTGCACATACATGTATACACACATGTATACAC
ATGCAATAAATACACTTTTCCTTTATAATCCTAATGACAGCCCTGAAGAGAGGTGTCAACCCAACACCTCCATCTCACA
GCTGAGGAAGAAGAGAACTGCACAGGGCCGAGTGCTGAGTAACTGACAAAGCAGGAATGCCCACTCAGCATCTCCCTCC
CAAGTCCCCCTTTTCCCGTGACCACAGAAGGACTCTGTCTTCTACTTTCTCTCATCCGTGACCTGCAGGCTTGGGCTCC
ATGGTCTCAGGGTTCTTTTCCCAATCTCAAATTCTCAGCTCATGCTAGCCATCATGGATTAAGATGAGACTAGGAGCGA
GCCAGTTGGATCTGTACTGTGCTTGGAGAGTTTCTGTGGACCCAGTGGAAGCAATGAGATTCTTCCATCAGTGTCCTGC
TGGCTTGGGTTGGAGGACCTTGGTGGCCTGGGTCTGCCTTACTGTTGGAAGTTGATTAAATCTTCTCAGGGGTGGAGAG
TGAAACTAAGGCAGAAGTCAACTTGGACTTCACTGTGGGGGTGAGGGGGGTGGGCTGATACTGTTGGGGGTGGGCATCT
TCATGAGCTGATCTTCCCAGCTCCTTTCCATTCTGGGTCCTGAAGCTCAGCAGTATACCCAAGAGGCTCAGGTCCAGCC
CAAGGGTGAAGCAAAGAGAGGGAGCCAAAGGACTTGTGAGGAACTCTTAGAACATTCAATGCGATCAGTATTTGGTGCT
AATGCCCAGGACTGCTCTTGGCTTCAAGAGATGTGGTGGACTCTCTTCTCCTATCTTAGGGTTTCTATTGTGGGAATGA
AACACCATGACCAAAAAGCAAATTGTGGGAGAAAAGGGTTTATTTGGCTTAGACTTCCATATTCCTGTTCATCACCAAA
GGAAGTCAAGACAGGAACTCAAACAAGGCAGGAACCTAGAGGCAGGAGCTGATGCAGAGGTCATGGAGGGATGCTACTT
ACTGCCTTGACTCCCATGACTTACTCAGCCTGCTTTCTTATAGATCCCAGGATCACCAGCACAGGGATGGAACCATCCA
TACTGGGCTGGGCCCTTCTCTATTGATCACTAATTAAGAAAATTCCCTAGAGCTGGATCTTATGGAGGCATCTTTTCAG
TTAAGGTTCCCTCCTTTCAGATGACACTAGCTTGTGTGTCAAGTTGACATAAGTCTACTTTAGCACACCTCCTTAGAGA
ATTAATGAGCATCTGGGTCCTGGAGGAGCCTGGGGTCTTCTTGACAAACCACATATGTTCAGCAGGGTGTCACACCAAA
TGTCCCTGGGATATGGGCTAGCCTAGAGCACCAAGAAACTTGGGTAAGATACAGAGGTCCTCAGAAGGGGTCAACATGG
AGGTTCACCTTTGTCACATGCCTCACCTCAAGGCCCTGGGTTCTAAATAATAAATAGGTTTCCACATCCTGGAAGGGCT
AGAGAACAATGTATCTGGAGATAGGGCCTGACCTGACTAACCCCAAGAATTAGATTTTTCCCCCTGAAAACCAAATGAA
TATTATATTTTGTTTCACAAATCATCTATTTCTACAAGTCTGTTCTTGTTCCCCAAATCTTGGAGTAACCTCAACATCC
AGGGACCTGTTTTGTGTTTCTCAAACGCAGGGTCCCAGAAGAAGCCCGTCAAATTTGGGGACTACCACCTAGATAGAGC
TCAGGAAATAACTACTTTATCTGGCTAGTTCACTCAGTCAGCTTGTTGGCTACAGAGGGACAGACTCTCCACAGTTTAA
ACAGGATGGGAATGTGGGGTGTGGGTCAGGACATTTGAGCCCAGTGGGACACTGGCAAAGTGGGACCAGTTACAGAATC
TGGTGGGGTGCCCCGGGTTGGGGGAAAAGGGCACGTTGGTAGATCGCTACCAACATCTACCAGTAGAAACGACACAGGA
TTTTCCTGGTGTCTTTTGGTTGCCAAACAGAACCCTAAACAGATCTTAACTGACCACTTTCTCAGTCAGCTTCAAACTG
TTCCTTCCTTGCGCCTCGGGTCCTCTTCTTTTTCTTAAACTTACTGTTTTTCTTTGGCTCACAATGCAGGTGGAGGCAA
TAGCCAGGAGCTGAGCCAGGAGTCTGCAGTTGATTGACTGGTTGTGGGTGAGACCTTTTCTAGCTAGGCACTGCTGTTC
TGTCTGGCTCTCCGAGAAGCTCCAAAGTTTCTGAGATGGATTGCCTGTATGTTTGCTGGTTCCCAGCCACCCGAACAAA
CCACCCCCGTTTGCCCTGGGACACAGGACTTTCAGTTTTAAAACTGGAAATGTTCTAAGGAAATTGAAATAAGTGAGTC
AACTTACTGCCTCCCATGTAAGAATAGCTTACGAAAACAAGCTTCTACTCTCTGACTACCAGAGGCAGCAAGGAGGGTC
GCAGATGATGTTGCAGGGTAGAAATGAACCCCAGAGGCTCATGGAGGCTTCCAGAGAGCAGCTTATCATGGGCCAGATC
TTGTGCCTTGCAAGCCTCATTAGTCTCTCCGATTTACAGGTGAAAGCCTTTCTCCAAGCTTCCTACGCCCATTGTGGAT
CATGGTTCATGACTAGTCCATTGCGTAGAATCTGTACAGATAAGAATATGGACAGATAAAGCTCCTGGTCTGGGGAGCT
ATTGAATTTCAGAATTGACTGTGATGTGCAAACTCATCTCCTCAAAGATGCAAAAATGTGCTGATGTCTCTGTCTTTGC
TAGCTGGCTCATTCACAGGATAGCCTTCTTCTACAACCCAGGCCCACCTGCCTTGGGGATGGCACCGCTCATGGTGGAT
GAAGTGCTCCACATCCACCGTCATTAAGACCATCCCTCACAGGCATGACCAAGTAATCCCTCAGTGGGAACTCTCCTCC
AAATGACTCTAGGCTTATCAAGTTGATAGCTGAATCTAACTAGGATATAACCTATTATATACTTAATGCATAGGTATAT
AAGATTTATAAGGAGCAGGGATAGCATTGTTCATACTTGTTTGGTTGGTTGTCTTATTTGTACTATATTTTAGTCTAAT
TTCGTTTCATTCTTGCATTTTTTCTTTCTTTTCTTTCTTTCCTCCTCTTCCTCCTCCTCCTCCTTCTTTTTGAGACAGT
ATCTTGCTCTGTGGCCCAGACTGAGCTGAAATTCACTAAGTTAACCTGGTGTACTTTGAACTTGCAATAATCCTCCTGC
CTCTGCCTCCCAAGTGTGATTACATACATGAGCCACTGTGCCTGGTCTTGAACATTCAGAGTACTGGCCACAGTTCCAA
CCCATTCTTAGTAGCCAGTACAAAATTCCTTCCATTTGGACCATGTGCGTGGTGATCCATAAAAAGAAAAAAAAGGACA
GATATTCAGCTATGACTATAATTATCAATATATCTGTGCTTTCTGTAAGAGTGCACACCCACAGTGCACACATTTAGTG
AGCTAATGTGAACAAAGCTAGGTCTTCCAGAACGCTGACTGATGAAGCCCTTTCCCTATGCCGAGAAGGCAGCCTGTTA
ATTCCTACTTTATCATGGAGGAAACTAGGAGGAGGCCTATATGAACTGCCACTCACACAAGTGGAGAGCTGAAAGTATC
GCTTTCTGGGGGTGCTGTCTTGAGTTTTTCTCCCCTCTACAGCTGCACATTGTCCTCCATAAATCTCCAGTCATTTAGC
CAGAGTGGTACCTAGCCATAGCGTCCTACCAGAGATGTGGCAGAGCTAGTGCTGTTTTCTAAGTCAGACATATCGACTG
TTGTCCCAGAACTCTAACTTCCTCACGTTTGCCTCTAGATGCACATTTCCTCTTTGGTGTTCAGCCAACACCAACACTC
CATCCAGCACAGGACACTTAGATGGAGTGTGGACGTGGGGCTATGTCATGGCTTTAAAAATGTTGATTTCTAAATGAAA
GAATTAGTGGAAACCCGATCATTTTCACAGTGTCTTAGGCTGTAACTCATTTGCTTAGAAACTCAAAGGGAAGGACTAC
TCTATCATCGGAGTGTCAATAAATAAACTGCAAGATGTCTGTGGAATAGTCGCAAATCTATTGTGTGTACATGTCTTGT
TGGGAATTGTACTACACATCAATTTTAAAGTTTTTTTTTAACTTCTTATTGATTCTTGTGAACTTCACACCATTCACCC
TAGTCCCACTCATCTCCCCATCCCTTCGTACCTGCCCTCTTCCCTTGCAACCTCCCCTGCTACAGAAGCTGTAGTGTGT
CCCAGTGTGTCCCAGAGTACACTCTTTCGTCCATACTTATTTATTTGCCAAAGTTCATTGCAATGACTTTTTGGTTTGG
TACAAGGCCTCTGACTCCTGCTACTGTATCAATACTGGAACCTCTCTGGAACTCCTCTTAGATATCCTGTTGTTGCTCT
GTGTCATGGTGATCCTGTAGTTTTAGATCTGTAGGACTGACCCAGCCATTCATGCACTCCAGCAGTTCATCAATGGGGT
AGATGTTGGGGTGGGACAATTCAAAGCCCTGGATCTGGGCCTGAGAGGTACCAGAGCTGGTCAGCCTTCTGGCTCTCCT
CCTCTCATTGCCCTTGGAGACAGCTCACCAGCGTCCCCCATATCCAGACCAGCTCGGCTGTGCTGCCCAAGGAGGTGCA
GGGCCTGCTCTCCCAAGTGCTGCAGCAGGGGAGGGGCAGGGCCAGATCTCCTGCTCTGATGACCCAGGGCAGCTCTCCT
GCTGCCAAAGGTAACAAGGGGTGAGGGAGGGTCATCTCTCCCTAACCAATGTCACTACACAACAGGCAAGGGACGGGGC
CAGCTCTCCCATACATGCGCCCTTGGAGCTGGCTCACCCACAACCCTGCTATCAGGGGCAACTCTACTTCGCTGCCCAG
GTGAGGTGGAGGGCCCAAGTGCTGCCGCAACATGACCTCCTTTCATGACCCCAGGGGCAGCAATCCCATGAAGCCCAGG
TGAGGATAGCGTCAGTTCTACACAACACTCAGACATCAACACGTCCCTGGGTGGTATCCCAGACCAGGGACATCTGCCT
AGCTGCTGCAGGGTTACAGACCAAGATGTGCCCTCTCCCTGTGACAGCATAGGCCAGGGCCTTGCTATGGTCCCAGGTG
GCACCACCAGCTGCTCACATCAGGTTCCTCATGACCCTTGAGTCTGCAGTTCTGCCTTTCTTCATTGTGCCCACATCCT
TCTGTTTCTTGTTCTCTTCCTTTTCTCCACCACTGACTTGCTCCTCTTTGTGGTGTGTGTGCCTGGAGACTCTGAGTAT
CCGGGGTCACCTCAGGAGTGCTGTGCCCTGATTGTGCATTATGGTGCCAGGCAGGGGTTATCTCGGGCACAGTCTGGCC
ACTGCTGGACTAGTGGACATCTCGGGCAAGATCCCTGTCCAGGCTTCATGGTGCCAGTCTGGTGGTCATCTTGAGCTCA
CTCCTCTCCCAGCCTGCCAGAGTGGCCCTTTGCAAGGGTCATCTGTCTCAGGCTCACTGCCACCTGGGACCCTCAGTCC
CAGGTGGGATTCTTCTAGTCTCCAGCTCACTCTCCATCCTGGGAGCCCATGTGGTCCTGCCTGGCAGCGGACTGGTGGT
CATCTCAGGCTCAATTATTTCAGGAAGTGTTAGGCTACTAATCATTCAGATGTTCATAGGTCAGAACACCAAGCACAGA
TATAATCTCTTCTTTTTGCCACCTACTGATATGCCTGTGACAGAGCAGCCATGCCTGTCACACCTCTAGGGACAGGGAA
GCTTTCTAATGATTTATTTATTTATTTGTATTTTCTGTGCACTGGTGTCTTGCCTGCATGCTTGTCTGTGTGAGGGAGA
CAGATCCCCTGGACTAGCATGACAGACAGTTGGGAGCCGCCAAATGGGTGCTGGGAATTGAACCCATGTCCTTTGGAAG
AACAGCCAAGTGTTTTCAACCACTGAGCCATCTCTCCAGGCCCCAATCTTAATGTTTTAAGAATAAATCAAACAATGGA
AAGGAGCTTCTCTGATGTTAAGTAAAATGAGGCAGGATGGACCACCGTATGGACCAAGGGTGTGGATATTTCTGCTAGG
GCACACAAAGCAAGGCAGCGCTAAGGAAGAACACCCAGACAGAAGTGGTTCTCCGCTGGGCGGGGACAGGAAAGCCTAG
CAACAATGTCCCTTTGTTCTTTATGGATTTGTATGTTCTTTATTGTCCTACAACAAACATGTCCTATATTTATGCTTAG
ATGAAACTATCAACTCCAGACTCTAAAGTGACATCCACCCTCCACTGCCAGGTGCACCAAGACATTACACTGGTGGTAC
ATGAAATAAAAGCCATGGAGCACAGAAGTGCTGTTTTCCAGGACTCTGAGAGTGGACCAAGGACGCTGCAGGATTGCAG
TGAGAGGGATGGGGTTGTCCTGTAGTGTGGTTGCTATTCCCTTTGCATTTTTACGCCTATTATTGCATTCGAGGCTCAC
TGCTTCCCAGAGGTGGACTCAATTCAGATAATGATACTTGAAGCCTTAAAGAGTGGTAAGAATGCAGAACACTCTCCAT
TCTGGAAAGATGGGAGACAGGTATATCCAGGAATCCCTCTCCACTCTGCCAGCACCCACTCACAGTGCCCAGCTCAGAG
CTTAGGAATCCAGCCCCAAGGGTTGGCCAAACACCTTAGATAGACGAGTCTATCTATCAGATCTGCATCTTGCCCTGTA
GGAAGGGCAGCCAAGGGCGAGGCAGCAAGGACAAGAGCTGCCACTGGATCTGCTGAGTACTGACTGGAGGTCTAGTCCT
GTGCCTGGGGATTTATGCCTGGGAAGACATGGAAACCATCTTAGTGGGCTTTCCTGTGGCCATGTTAGTAAACCTGGAT
TGCCTGCCACATTGTTTGTAAGGCTGCACTCTGCAGTTGTTCTAAACAGCAGTGACCTTTGACTCTCTGAGGCCAAGAG
TAGTAAGGTTCATTTATTTGTGTGTGTGTGTGTGTGTGTGTGTGTGGTCAGGAGTCACCTGGGGTTTTGTTGTTGTTTT
GTTTAAGACAAGGTCTCTCACTGGCTTTGAATTCATTAAGAAGACTACCCTGGATGGCCAGTGTCTTAGGGTTTTACTG
CTATGAACAGACACCACAACCAAGGCAAGTCTTATAAAGGACAACATTTAATTGGGCTTGCAGGTTCAGAGAGGTTCAG
TCCATTATCATCAAAGCAGGAACATGACAGCATCGAGGGCAGGCATGGTGCAGGAGGAGCTGAGAATTCTACATCTTCA
TCTGAAGGCTGCTAGCAGAATACTGACTTCCAGGCAGCTAGGATGAGGGTCTTAAGCCCACACTCACAGTGACACACCT
ACACCCAACAGGGCCACACTAACTACTAGTGTTACTTCCTGGACTGAGCATATACAAACCATCACAGCCAGAAAGCCCC
AAAGGTCTGCTTTCTCAGTGATGGGATTACAAATGCACACCAACACGCCCAGCTGCTTCTTCCTTTTGTTCTGAGGATA
AGCCCAAGGTCCTCATTACTGACTGAGCTAACTCCCCATCTTCACATTCTTGGTCGATTACTATCAAGCATTGGCTATT
CAGTGATGAGTAAAAGCAGAAGAGCTCTGTCCCTCCAGTCAGATAAACAGTGCACAGTTATGCAAATGACAGCAAAGTC
ACCAGGCATGTCATATAATCGGGGGAGGAAATTGCCTGTTTAAGGTAACAAATATGGTGGATTCTGCTTTACATATCTG
GGTCATGGACTGTGAGTTTTAGCGTCATCCTCACTGTCCTCATACAGAATGACTGAGTGGGAAACACCATTAGGAGCAG
AAAAAATGTCTCTGCCTCCCAAAGGCTGTGGTTAGATGGCTGGATCATAATAAGTTCTAGTGCATTATGGTTTCCATCA
ACAAGACAGGCCCATGTAGCACAAAAGACCTCTTAATGAAAACACTTAAAGGTGCTTCATAAAATAAGTAAAATCAGAG
TTCCCATGTATAGCTGAGCTTTCGAGAAAGAGAGGGGAGTGGTCAGGTGAGAGAAATGAAGAAGGCACAAAACAGCAGA
AACTTGACACACCAGTCACCGCTGTGGGGGACAGATGGGAATGGAGCATAGTGGCTATGAGAGCAGGAAAGAGCCCAAG
AGACTGTCTGAGAAGGATGTGGTGTTAGTTACAGTATGGGTACCTAAGATGACATGAGAGGCGGTGGAATCCCATGTAT
AAGCTACACGGAATTGCCAACTGAGGTACCACATGATGGACAGACTTTATACTATATAAAAGTTAACTCAAATGGATCT
GAGACTTAAATATAAGATCCAAAACTATAAAACTCAAAAGAAGACATGGAGATCAAGCTTTATGACACAGAGCTGGGCA
AAGCTGTCTTAGATGTGATGCCAAAAGTATAGGCAATATATATTAAAATTGTTAAAAGTTATATGATGTTGAATTTTTA
AACATTTCTGAGTGTCAAGCAAGACAGTCAATGGCATCTTAGAAGAGGCAATCTATGGACTGGAGAGATGGCTCAGCGG
TTAAGAGCACTGGTTGTTCTTCCAGAGGACCTGGGTTCAATTCCTAGCACCTACATGGCAGCTCACAACTGTCTATTCC
CCAGCTCCAGGGGACACTACACCCTTACATAGGCATACGTGCAAGCAAAACATAAAATAAAAATTAAAAACAGAAAATG
CAATCTAAGACATGGGAGAAAATACATGCAAATTTATGTGCAGTAAGAATTAATATCTAGGAGCCAGGCATGGTGGCGC
ACACCTTTAGTCCCAGCACTCAGGAGGCAGAGGCAGGCGGATTTCTGAGTTTGAGGCCAGCCTGGTCTACAAAGTGAGT
TCCAGGACAGCCAGGGCTATACAGAGAAACTCTGTCTCAAAAAACAAAACACAAACACAAACAAACAAACAAACAAAAA
AGAATTAATATCTAGGACATATAAAAAATTTCTTCAATCCAGCAACAAAGAACAACTGGAACTGTAAACTGAATAAATC
CTATTCTTTACTTTGCTTTTGGTTCAGGTACTTTTATCCCAGCAATGGAAAACACACTAGGACATTACAGGAGGAAGAT
CTGAAAGCAGAGGCTCTAAGCAATAGTTAAACACCCTTGATCATAGCAGCGCTAAGGTAGAAGGAACCCAAGTCTCCAC
CAATGAATAAACAGACAAGCAAACCAGACATAGCCACAGGGTGGAACATTAGTCAGCCTCAAAAGGAAAGACATTCTCA
CGCATGCTATAAAATGGACAGACCTGGAGGACGTTCTGCTCAGAGAAGTATGCCAACCACAAAAGAGCAAAGATATGAT
CTCATTTAAGTGAGGTCTGTAACACGGCTAAATGTATAGATAGGGACCGTGAATGGCAGTTTCCCAGGGTTGGAGGGAT
GGTGGGAGAATGGGGAACTCCTACCCAATGGGTATTCGTTTCAGCATCACAAGATGAAGTGTCTGAGAGGCAGATGATG
GTCATGGTTGCACAGCCTTAAATGTGTGATGCCACAGGAGTTGGTCCTGGAAATGGTCGTGATGGTTGGTAGATTGAAA
TGTGGTTTTCCTCCATTGGAAATATGTATGGGTGATCAGTAAAACATCACTAGACACAGGCCTCCCAGGGAGGGGAGAA
TAAAAGGTAGACTTACAAAATGATAATAAAACAAAATGTAATCAAAGGCTGTAGCAACGGAAGGTTGCGGAGGCAAACC
CCCTTCGGTGGAGCCTGATATTATAACTAAACCGAGAAAGAACTGCAGCGCATGCAAATGTCCTACCCTCATCATTTCC
ATTTCAGAGAGTCTCAGGTTGGATTAAAAAAAAGAGAGAGAGAGAGTCAAATTGCTTTTGAAAAGGGACATCCAAGTCA
GTGGGATGTGAGGTCACCTACATTGCTTCATGTCCCACCTCTCCCTCCATTCTCAAAGATGGTGGCTTAGCATCTTGAA
ATCTATCAGCTCACCCTCTCTCCTCTCTCATGAAGACTTTTGTCATTATACACACATGCACACACACACACACGCACAT
ACGCAGCCATGCATCTTGAATAAGCTCCCAATTTAACATCATAGTCACGCCTTCAAATTTCATTTTGCTACATAAGCTA
ATATAGTCAAAGGTTACACCTGTAACCCCAGCATTTGGGAAGTGGAGGCAAGAGGATCAGGAGTTCAAGGCCAGCCTTG
GCTACATAGCAAGCCTGTGCTACGTCAAACCTTGACTCTAAAACTAAATAAATTACCACAAAACCAAAATCCACAAAAA
TAAACATTAAAACACACAGAAAGTTTGAAAATCAAGGGGCAGAAGATTACCAGGAGACCAAGATGTAAAGATGATTTAT
CCATTTTTAATAACAAGTACAAGTGCCTATAACAAAAAAGAATGCTAGACTGTGGTATATTTGTAATATTTAAAAATGA
ATGAGACTCATAGCTACAATGTACCCAGAACAGAGCCCACAAAATCCAGCAAGGAAACAGTGGCAGATGTGACCAGGTG
GATGAGAGTATGTATCTCTGAATGTCACTGTCATTTTGTCTCATGAAGCTCAGAGTCACAGCCAGCAGCCAGAGCATGT
CCATTGCGGTTCATTTAAATGCAATTTGAAAAAAAAATCCCGGGACAGACTCTAATTGGCTCTGCTGGGTCACATGTGG
CCCTCCCAGTGCAGGACACCATACTGGTATTGTCTTCTTGAATCATGGGTCCAACAGGGACCCCTCTAGAACTGTTCTT
ACAAACAAGGTGGGTGGATAGGGCAATCCAGACCTGCTGTGTTAGGAAGGTGAACAAGTGAAAGAGACGCCAAAGAAGA
GGTGCGGTCCTTGGTGCCGTGGGAGACTCCTTGGGTGTCATGGTAAGCAGATTGTTTGAGCAAGGAGCAAAATGAAAGG
GGTTCGCCCCCTCTGCACTCTGCAGTCACAGCAGTTCTAGGGAGCCACCAACAAAACCACCACCTTAGAGGAAACCGGA
AGTGAGGCTTAGAACACTTCTCAATTGGCTACAGCTCAGCAGTCAGCCAGACGGCTGGCTACGGCGTAATTAATTGGTT
AACTGCAATGCCCCTCCCCTCCAAATGTTATTTAACATCCTTTGCTGCGGAGAAAAGGTCCCTCCAGAAAGAGGGCAGT
CAGGTACAGGCAAGGGATCTTTCTGAACTGGACTCACACCTGTTTCTTATCGCCGCTGTAACAAATGATCCCAGCCATG
ATGCTTAAACAAGGACTTACTGTAGGGAAAGCCTTGGAAAATGTGGTGCCAAGTGATTTCCTCACCTTCTTCCACTTCT
ACAGTCACCTACATTCCTCCATCCTCAAAGATGGCAGCTTAGTGTCTTGAAATCTCTCCCCTCCCCCTTCTCCTTCTTA
TCTCATAAAGACCCTTGGAATTATATTGCCCACCGCTCTCCATGGATCTTGGATATGCTCCTAATTTAAAATCCTATCC
ACACCTTCAAATTTCCTTTTTGCACATAAGCTACTACTATGTTCAAAGGTTCTGAGGGCCCCCTTCTAAATAGGGGAAG
ACATTGTTTTGTCCAATTTGGGGTTACTCAAACTAAAGTCAGTAGAAGGCATTCAACACATATATTAAGATATAGCACA
AGGAATTGGCTCACACAATATTGGAGGCAAGCAGTCCAATCACAAACAGGCTCTTAGGATTCACCAACTTGGGACAAAG
CTGCTGTCCGTGGTGTGTCTGTGTGTGTCTCTGTGTGTGTGTGTCTCCTGCACACACTTGTCAGTTCTCTTAAGTTTTG
TGGTTGATTGGCTTGGGCCAACTCAGATCATACAGGATCATATAGATCATCTTCCTTCCTTCAAGTCAGGCGATTCTGC
ATTTTAATTACATCTGCAGACCTCGATTGTGTTTGACTAGCCAGAGGCGAGGCTAACCATGCTGGCCCATCCAGTGTAC
TGTTACAATTAGGACCTGTTGGGGATAGACTGTTGCAGCTCCAGGAGTTGGGGCTACCATGGGAACAGAGGGAATCCCC
AGAAGGTAGCGCCTTCTTGAAACTGTTGATTTGAAGAATGCATGCTTGGGGTTGGGGATTTAGCTCAGTGGTAAAGTGC
TTGCCTAGCAAGCCCAAGGCCCTGGGTTCGGTCCTCAGCTCCGGAAAAAAGAAAAAAAAAAAAAAAAAAAAAAAAAAAA
AAAAACTTTAAAAAAAAAAAAAAAAAAAAAAAGAAGAATGCATGCTTGAGTGAACTGTAAGAAGCCCAGGAGGGGGTGA
GCACCTTCCCACAGTTTGAAAAAGCTGTGTCGAGAGAGAGAGAGAGAGAGAGAGAGAGAGAGAGAGAGAGAGAGAGAGA
GAGAGAGAGTCTGCTTCCTTCTACACCAACTTTGCCAGGCCACACAGTGAGCGAGCTACCTGTGACTACAGAGATGCCC
CTTCCTTCTGTTGCCTTCCCATTGCTAGGTCGCAGGCCCCTGGGGTGCAGACTTGAATGGGCGGGTGGTGGAAGGCCGG
TGAGCTCATGTCTGCTGCTCCTGGAACAGGAGGCAGCTGTAGGCGTGAGTGTTTGCTTGGCTTATCTTTTCCCATCTGA
AGATGGTCATTGGTGATTCATTCCGGAATCTTGGAGTCTGCGGGGAGAAGTCCCAACCCAAACCAACCAGGTGCAGGTA
AAGGGACACCCATCCCCCTGTGTCCCTGCACTTGCCTTCTAGTGTTCTAAGAGAAAAAAATCGGCCTGGGGAGGGGGCT
CCCCAACACCCACTGTGCAGGCAGACTCTCAAGAGGATTCCCCATGCCCAGATAGATAGCCCAGAAGCGGGAGAGAAAT
CTGTAAGTCCCAGCTTCTCACTCCATGAGAAGTTTACATTTTAGCAACTCTGTTCACAAAATGTGTTTGTCCAGAGGTT
GAGCTTTGTCACTGGGAAAAAAAAGTTGTCAGGGAAGAAGGACTTAAGGGCTTGGGAGTTCTTCCTTTGACTCGACAGC
CATGATTGTCAAAATGGAGTCTATGAGCTGGTGGCACCCTCCACACAAGCCAGCTTATTGTAAAGACAAATTTTCATAC
CCCATCCCTGACTACTGAATGACTGATGGAAAGACTTCTCGGGTACTTCTGATGTGCTCCAGCGTTCAGTGACATCTCT
CGGGCAAGCACATCTTGAGCCCCTGTTGTGCATGCAGAGTGTGCTATGGTTGCTGCTATGAATGTGGTGGTGAAAGTGA
ACAGCAAGAAGTAATTGGTATTCAACTCCCACGGTTGCCAGGTAACTCGACTGGCTGATTGTAATGGATGGAGGAAATT
TGCCTGGCCTCTGAGGTTGATGAGTAAAGGCCTCCCAGAACTTTCTGGTGTGGCTCAGGTAAAGGCCAAGGACTGTAAT
CTCCTGTCCCAGAGACCTCTCTGTGCAGGGAGACTGGAGTGGGACTGTATTAAAGCAGAACCTCCTTAGCCAGGAGGAA
CAGCCATGTTGCAATCTGCAGGCTTTCTGGGGGGGGGGAGGGGCTCAAAACACACAGGCTGGGTTCCATAGCAACCTCA
TTTAAGCCATGGGAAAGGACCAATTGTCTGGGAGGCTGAGGTCAGGAGTTCAAGTTAAATAGTCAAGGTAAGCAGGGAT
CTGAGAAATGGGCGTTCTCTTCTCTGTATCTACTCCTGGCTGCAGACAGTTTGTGGTGCTAAGAATGCCTGGTGCTCTG
AATGAGCCATTCCAGAGATTGTGGGAATCCCTATGGCCATTCCCTTGATGCTCTTTGGGGCCGTGGTGTGAGTGTGGGT
TGGGAAGAAGCACCAGGGGGTTGTGGAGACCAGGGTGCCATATTTTTGTGCATGTTACGAAACTCACATCTTCCCGCCC
TCCTTTCTTGACCTCTCCATTTGTCTTTTATTTTCTTCTGTCACAACAGCCTAGGACCTGATGAAGCTTTTCTGGGAAA
GAACTTGGTCTCAGTTCCGTGGTCTGACTGAAGACCCCTCCCTTTGAGGTTTCATTCAGTTCTGTCATGCTCTCTCTGT
GTTGACCTTGAAAATATTGCTTTCTCTTTTTCTGTTGCTCCATAGCTCCCAGCAAAAAGTAACCCACAGTTAAGAAAAG
GTTTGGCTTGGCTTATTCTGGATTGAGGGTACAGACTGGCATGGCAGGGCTGGCATGGTGGGAGTGTGCAGGAGCTGGC
TTTATGCACCTACAGTCAGGGAGCAGAGAGAAAGAGATAACCATCAATGCTCCATTTTCACTCTGCTTTTGGTTTAGTC
TGGGACCCCAGGTAATGGGAGAGAGCTACTTCCATCCTTCATGGTCTTCTCTCTATAGCTCAACCTCCCTGAAAATGCA
TAGCGATTTGTCTCTTAGGCAATTCTAAGTCTGGGCAAGTTGATAGCCAAGATTAAGAATGTGTGTGTGTGTGTGTGTG
TGTGTGTGTGTGTGTGTGTGTGTGTGTATGTATGTTTATATATATATGACATGGCAGCTTTGGGGGAGAGGGGCAAGTA
GGGTCCATATTCTGAGAGTGAGCACAGGACTCCAGAGCACCGCTTGGCGACCAGCCCTCCTGAGAAAGCTTCTACTTCT
GGGGAGGATCACTTGTGCAGACTTCTACCCATGCAAAGACTAGACCAGAGACTCAGCTTGGAGCACATCCCAAATAGCA
TTAGAGATCAAGTGGCCCTTGTCTGGCTGCAGATAACCACCTCACACATCAGAGTTTAGATGTGTTAGGGTGTCTGTCA
AGTAACTGGGGCTTTGTCCTGTTTTTTTTTTTTTTTTTTTTTTTTTTTTTTTTTTTTTTTTTTTTTTTTTTTGTGCCGC
TGTAGCACCACAGCTGTCATGAGGCACCCAGGAGGCTCTAGGTCAAGTAAGCCTTCCACCTTCTTCATGGGGAAAATGG
AGTCTGCCAGCGAGGCCCCCCGGCTGCGTCGAGCAGGAGTAAAAGCTAATAAAGATAAAGGAATCAGTCATGGGTGGGG
ATATAGCTCATCCTAGTACGCACAGGCTCCAGGGCTTGATTCCCAGCATTACCAGAAAAACAATGAAGAAGGAAGGAAG
GAAAACAGAATGGAGAAAAGATTTTTTCCCTCCAGCTGGAACGCGCTCAAGGGGCTTCAGTCCTACGGAGCCCAAGAGG
GACTTATTTCAATGCAGCCCTGGGTTCCTGTTGTCCACTGGGCTTGAGAATTGCATTCCACACTATCCCCCCACCAGAA
TGAGAAGTTGAATAATGAGCAGAATTGAAGTCCTCTTGCCCTGGAACCAGTTGCTGGCCTGGCCGGGACCTAGAGGTCA
CTCCCCGTCAGCCCTGAGCCATCCATTCTCCAATCGTGCCACCAACCATTGACAAGACCAGAGAGAGCTGGTCTTGTCC
TTGGTAGGCAGTGGTGCCTGCCCAGCTTTCCTCCCATGCCAGGCCTCTCACCGATGCTTGCTTTCTTTGCCACCCCTGT
TCCCTCCTTTCATTCTCTCTACGGGTCCCTCTCCCCTGACCCATCAGCACACTGACCTCTGCGCACCCGCTCCCACCTG
TTCCCCCGCAGCCCATTGGCTCCTTCCTCTCACCACCTGCCCTGCCCTGCCCTGCCCATGTTCTGACCTCCTCACTTCC
CTCACACCACTCTCTCCGGTGCCCCTTACCACCCCTGGGCCACCTCAGCTCTCAGGCCTTCCCTTGCCAGAATTCCTCT
CCCTTGGCTGCTCGCTCGTCCTGGCCGTTGTCCAACTAGAGACTTCCTGCCTTGTCTTCGCAACCTCGCCTGACTGCTT
TCCTGCGTTCTGCCCTGTTACATGACAACCTTTGCAGGAGGTCCAGCATGTGTCATACCTGCTTAGAAGTCTTCAGTGA
TGACCAGATGGAGATGAACTCCTATGGCTCCCCAGGGAGGCAATTGTCACTCACAGTGTAATTTCTTCCTGCCACACCC
CTAGAATGTTCCCCAGATTCCTCTGACCCTATCACCGTGTGACAGATGCGCAGCCTGGCTTCCCTGCCACACAGTCACC
TACAGGGAAACCTCCTCCATGCCTTTGTCACTTCATTGGTAAAGTCAGACCATCAATGGCAAGCGCATCTCATGAACCA
CCAGAGGTCATGTGGTAGAACTGCGCTCACAGCCTAAGGCAGAGAAGCATTTCCTTCTCCGGAGGTGTCTGATGCAATG
TGGAGATCTGAATGTTGGGTAAGGGCTTCTGTCTCTTCCTAGCATCCTCTTCCTTGCCCAGGGGAGAGAGGGGGCAATC
TGCTCTGGCTTGATGAACCTGCTCTGCACAGGACCAAGCTTTCAGGGGACTGATGTCATGTTGAGTAATGGGAGAACGT
GTGGAGACTGCCGCTAGTTCTGTGGATCCTCTGGCAGGCTGGGAAGGGGGATATGAGGTGACCATGATAGAGCATGTTG
TGTCCCCATGTCAGAACAAGTGTGTCTGGTTGTGAGTACTGATTATGGCGAGGATCCTGACCTGTGATCAGTGAGTGGG
GGGAGTGCTCACCTGTGGGTATGGATGTATAGACTCTGCAACACTGTCACCTGGAGAGGGACCTTTACTTAGGATCAGC
CAGAGGAGACAGTGTTGACAGTGAGTGGGGCCAGCAATCCACTCAGTGGAGGTAGGCAGGTGTCTGCACACACACACAC
ACACACACACACACACACACACACACACAGGGTCCTCAGGTCCTCTCCTCCATGTTTCTTGTACATCTCATTGGGGACC
TACTGAAGCTTTGTTCTCAGACCTCCTCTGTGGCTTCCAGCAAGTATGTTGCAGTTTTTCGGAAGGTCTCCTGGGAGCT
GCTGTTGATGAATGAGTGGTGGGTGGACACAGGCTGCCCTGGGCAGGGGTTTTCCTGAATGAAAGTTCTTTCCTGTCCC
ACCAGGGCGCTGACTCATGTCTGATGGGACTTGCTCCGCTCTTGCCTGGGTACTCCAGGGGCACAGCTTAACACTCTGT
TTCTCATGCCCAGAAAAAAAAAATTGACAGCCCTTTGTCTGTCTGCCAAATTAAACCCACAATAAAGACCAAAAGTCAA
AACTGAGTCCAAGGCTTCACTTCTCCATAGGGAGATGAGTTTGTTGGGAGCTCTGGGCTAGAAAATGACTGTGGGATTC
TGAAGAGACTGAAAGAACCGATCCACAAGAATTGATGACAAAGAAATTTGTCTTTGTCTCTAGAACTTTTAGGGGGAGG
CATGGCTACAGGAAAAGGAAACCACCTGCTTCTACCGTGAATTTACAACCTAATTTGACAACTTTGTATTTGTATGCAT
TTAATGCTAGCTTTTTGGTGTCTGGATAAGAGTGGGCCCCTCACTGACAGTCACTCCTTGCCTTATCCATGGTCTTTAG
TGTTCCTGGAGAGGATCGCTTCTTCCTCATGCTTACCCTGCCTCTGTGGTCATTTCCTTCGCGCCCTGCTGCTGTGTGC
TGGATGCTGAGAATGGCTAGGGCCCTGAGCCCCAGTGATTGAAATCACACTCTGGGCTCAGTTGCCTGAAGGAACAATG
GAGAGCTGTAGCTCAAGGGGCCGTCCTTGACACTAGCCTGCTGAGTTCATGAAGAAAGGAGCTGGAGGCTGGGGGGCAG
ATCAGGCGGTGAAGAGTTTGTCATGTTAAGCATGAGGAGCTGAGTTCAATCCCCAGAGTCCACACTAGAAAATAACAAG
GATTGAGGAATCTGGAGGGGGCAGGGACTCCACAGGAAGACCAACAGAGTCAACTAACCTGGACCCTTGGGGGCTCTCA
GAGACTGAACCACCAACCAACGAGCGTACCCAGGCTGGACTATCTCCATCCCCTGCATAGTTGTAGCAGCTGTGCAGTT
CCCCCAACAACTGGAGCAGGGACTTACCCTGACTCTGTTGCCTGCCTGTGGATCCTGTTCCCCTAATTGGGCTGCCTTG
TCTGGCCTCAGTGGGAGAGGATGTGCCTAGTCCTGCAGTGACTTGAGATGCCCGGGTGGGTTGGCACCCAGTGGAGGAC
TTCTTTCTCAAAGGTGAAAAGGAGGCGTGGGGGAAGGGGCCGTGTGAAGGTAGGACTGGGATGATGGGGGGCTGTGATC
AGGGTGTAAAGTGAGTAAATAAATAAATTTAAAAAAAAAAACAGTAACAAGGCCAGGCATGGGGCACATGCCTTTAATC
CCAGCACTCGGGAGGAAGAGGCAGGCGGATTTCTGAGTTCGAGGCCAGCCTGGTCTACAAAGAGTTCCAGGACAGCCAG
GGCTATACAGAGAAACCCTGTCTCGAAAAAAACAAAACCAAAACCAACCAACCAAACAAACAAAAAAACAAACAAAAAA
ACCCCAAAAAACAAAAAATAATAACAAAACCGAGTGTGGATGTGTGGGGTTCTTGTTATCCCAGCACTGAGTGGGAAAG
AGCGCAGACGGGAATCCAGAGGCTTGCTGGCCAATCAGCTCATCCTACTTCTCAACTCCAGGTCAGTGGAAGACTCCGT
CTCAAAGAAGAGGATGGCAGCTGTCTTAGTTAGGGTTTCGTTGCTGTGAAAAGATACCATGACCAAGGCAACTCTTATA
AGGGACATTTATCACCACAGCAACTGAGGTTGTTTTCTGGCCTCCAGACACAGACATGCATACGTATACACAGAGAGAG
AGAGACAGACAGACAGACAGAGAGGCTTGTACACAAATGTATACCACATGCACATACACACACGGGCGGTGGCAGGGAA
GAGGAAGAGGGAGAGAGGCACACATGCAAACACATGCACATCACGTGCATACTACATGTGCGCGTGCACACACACACAC
ACACACACACACAAGGAAGGAAGAGCCGTCAAAGAAGAAAGCATTTCTCACCATCATTTGTTGCCCCAGCAACTTCACG
CTTTCTATTACGGACACACTTCGGCACTGGGTTGTGGCCACTGTGCTGGGAAGCTTAGGCAGAGGGGAAGGCAGATGAG
AGTTTGAGTGTGGACAGCGCTGTAGGCTGTGGACTGGGCTCAGATTTTCATCCAAGATGGTGGGAACCCACAGGCTTGT
CACTGTCACAGCAGGCTCTGGTCTCCATTTTCAATCATGCTGCCACTTAAAAGTCCCTTCCCACAAAGCCCAAGGGCAA
ACTGACCCTAGCTCTTGGAACTCAGAAGAGTTTCTACCTTGGAGGTGAGGCAGGTGGCAACTGAAGGGACCCAGAGACC
TTTGGGGAAGCTGGTACTGATCTAGTTTTTATTATGGGTCCTTGAACTGTAGCCAGCTCGGGAATCTTTAAGCTGTACT
CTTACAACAGATGCATTTTCTGTGCGAATGTACTCCAGTAAGAGCTTTCTCAGAAGACTTCTCTGGCTGTTTAAAGAGT
AGATCAGAGCAAGGAAAGAGTAGGTATAGGAAATGAGAATAGGGATGTGTCTATGAGTCAGGGGATGTCACCAGCTGCT
GGTGTCAGGGACTGGAGGGGACCAACATAGATGAAAAATGTAACAAAATTTGAGATTTGGCTTGGAGGCACCGAGGGCA
AAACCTAGAATCCAGAAAACACTGTTGGAAGGCAGGAATGACGGGGCATGGACTTGATAGGAAATGGGTGGAAATAGGA
GAGGCACAGTTGAGAGGAAGTCAGCCTGGGGAGGGAGGGCTTCAGTTAAGCATCCATGCTCCCACAGGTGGGTAAACAA
GACACCAAAGGTTGGTACATGTGAAAGCACTGATATACATCCCGGGGACCATCAACATCTGGGGGATAGAGAAGACATC
TCTTATCAAGGTTTTACAATAGAAACAGACCTAGAGGAAGTGACATCATGTACTCCAGTGCCCATCCTTGTTGAATTAC
CTGGTACAGAGGTTCACATCTGTGATCCTGACTCTTGACACTTGGGAGGTAGGAGTTTGAGGCTAGCCTGGGCTATATA
GCTAGACCCTATCTCAGACAGAGAGAGTGACAGAGACAGAGACACAAGAGAGAGACGGGTAGGAGGTGGGATAGAAAGG
AGAGATGAGGTGGGGGAGTCCTATAGGACTTAGATACTTTTTGGAAAGCCTTATGTGATCGCCAGGTACTGTCAACTTC
AGCAAAAACAATCTCTGTAGCACCTCCTTCCTCTGAGCCCTCAGAAGAGTCAACATTGTCACACAATGGAAGAGTAGTA
TGACTTAGTGTCTGTTGGCTGTTGTCATTTGCCAACTACAATATGAGATGAGCTTGCAAGTTGCCCTGGGCCTTCGTCA
CCTTCTCCACAGGGTTATGTGAGATTATCTAGGTTTGCAAGCATTTACTGAACATGTACCATGCATTGGCTTACTGATG
TGACAGTGAGAGCATGCCTGGAAAAAAAAAAGCAATGGAAACAGATAATAGAGACAAAGATGATGTGACGGCCCCACAT
GCCCTCCCTCAGAGCTCCCTAGCGGTCATTGCCCACTGATAGGACCATAGGACCCCTCCTTGGAGGGACTCTTGTGAGA
CTGTCTTTGTATTATAGTTTGCACTTTCTCTATCTGAAAAACTGGCTTCCCTTTGCTTGAAGGCAGGAAGGGATGGGCT
TTATCTTCAAATCCTTCCAGGATGTGAGAAAAAATATTGACATCCCATCTTGTTACCAGAACAAAGACGAGGAAGGATA
TAAAATAGCAACGTTTCCATATTCACAGGGTCTAGATGTCCCTTCCCTGCCATAAATGCATTCAAGGCACGTTGGACAA
CGGCGGCTTCCTTTCAACCCAACAGGTGACTGCGAGCCAACTTCCCCCAGCACAGGATGGTGCTTGTTCTTCATTAAGG
AGTGTTTTCACTGTCTTAAAAGACAACAAAAGCTTTTCAGAATCTCTGGAGAAGAATTCAAGCATATATTGACATGAAC
ACTAGGGATAATTTTATTTCCAAGTGTCTATTTGCCCAGGAAAACGTTCTTCTAGTGAATATGATAAATCCATAGAGTC
TCACATGCGTGCATTTAACCTCCCCTCCCCCAGGCGATCTCTCAGCTTGCTCTTCCATGACACCTCGTCACCACCTCCT
TGCAGTGGTCTGCCTCACACACTCCTGGGGCTGAGCACCAAGCACAAAGCCCCACCCCCACACCGGGATGGGGGAACCA
GAGCATCTCGGAACATCCAGTGCTGGACGCCTTTTAGGGTTCCTAGTGTGGTTTGGGGTCCAGGATTCCCACTGCTTCT
CTCCTTTGTGCCCCAGATTTAAAATGAGGATGCAGCATGGAAAGCCACAGGCTTCCTGTCCACAGTGAAGTTCTACATT
TTCTTTTACTTCAAAGGACACCCCAGGACCCCATCTTATCAAAGATCCTTTTTAGTGAGATTATAATCCCAGATCCAGA
TTCCCAAATTGCCTTCTTCCTTCCCTATGAAGAGACAGGAGGTGTCTTATATCTTCCGTGCTCCTCTTTAGACTACTCT
GGTACTTTCTTTTTTCTCTTTTCTTTTTTCTTTTCTTTTCTTTTCTTTTCTTTTCTTTTCTTTTCTTTTCTTTCCTTGT
CTTGTCTTGCTTTTCTTTTGTCTTGTCTTTCTTTCTTTCTTTTACTTTCTTTCTTTTCCTTCTTCCTTCCTTCCTCCCT
CCCCCCCCCTCTTTCTTTCTTTCTTTCTTTCTTTCTTTCTTTCTTTCTTTCTTTCTTTCTTTCTTTCTCTCTTTCTTTC
TTTCTCTTCTAAGAACCATCAGGACACAGTGCTTTTCTAGTTCCTAACAATGCTAATGTTCCTGATGCTACCTTGGCTC
ATTTCTCCTCCAAAGGACATCAACTTTGAGCCCAGGCTGGCTATTTTGAATCTGAGTGTTATCTTGTGTAGACATTCTT
CCTGCATGGGAATATACTTGATTTCCTGAATGATTGTGTGACAGAGACACTCCCCACGGGGGCACTGGGCTATGCCCGG
CAGAAGGTACAGGAGGCTTAGGCAGGCTCCCTGGCTGTTCCAGGCCTTTCTCCCTAGGTCACATCTTTGTTTTACATCC
CACTGTCCCTTTGGAAAGACAGTAATAAGGATTCACCATCCATCTTCCAATTCTCTGGGCCCTCAGGGGGGTCTCAGAG
GCACCAACTCAGTCTCTCTCTCTCTCTCTCTCTCTCTCTCTCTCTCTCTGAAATGTCAGATGACTCTGGAAAACATCTT
TATCTGTGTTGAATAGGGTACCTTGAACCTAGGACCTCACCGTTCAGCACTTAACCCCTGGTCCACTGCCGGCCAGCAA
GGAGGATGAGCTGTGGGCAGGGAAGTTACGTGGGAAGGAGCACACACAGAGTCAGAATCACCATCAGGCCGAGTGCCTG
CAATCAGAGGCACACCTTGCTCTGCAAGACTCAGCTAGCGAGATAACAGTGTCATAACCCGAATGTGCTAGGGTTATGA
GGGAGGATCTGTTTGTACTTTGGGATTGACAGTTTAGTCCACTGCCCAACATATAATAAATGGTCTCTTAATGGTGTTC
TTGACACCACTGTGATTTGAAAAAAGCACACCCATACCACCACGCTGTCCTCCAATTTCAGTAGTGGTCAGTAAACTAT
ATGAGGTAGAGCCAACAGTTTGTTATGAAATAGGCTTTCTCTTAAATGATTCTGCTCCATGATAGGCTGATATAAATGT
CTTACCTGGCAAGTGGGAGAAACATAGACATGTAGAGCATTTTCTATTTATGAGAAGTTTATTAAATATGCTTCTTGGA
GCCCTTCTTTATTCATTGAGAAAGTAGGGGCACAAGGATGGGAATAGATTGGGTTTGTAATTCAAGACGGTAGACTAAA
CACATGCTCATTTCTCCTCAGCTCCAACATCCCCATTATGATGAAAACGGTGATTAAGGATGATTAAAAAAAAGGACCA
GAAACAGTTAAAAAGGGGGACACACTATGAATGGATGAAAGAACTCAATAATTTCTGTGGCATGGACAATGGTTAGTGG
ATGGGTCAGACTGGGAGTCACAGACTCTCAGGTCCCTTCAAGGGGTGTTCAGTGGTGGGAAACCTGAAGCCTGCTGCAA
CTCCAGAGGCAGCAGGGATAGAATCTGTGAGTATTATGAGGTAGAAAGCTGAATTCTTGGGGTGATAGTCAGCAGAGGA
CAGCCCTTTGCCCAGGGGAAAACCCAAGCGTCCCTTCATTGTGAACCAAGGGCACTATCACCAGGGGGTTTGGGGCACT
GGTTCCTGTGGCTTCCAGTTTGTTTGCCACAGAGTGGGGTGGTCTGATAATAATAAAAGACCGGCAGAACTGTGAACAG
CTGGGAGGAACAGGGAGAAGGACACATCTAGGTAGCCAAACCCCTATCCACGATGGTGCATGCTCTAAAACGGCAGAGC
AAGACGTCCTAGAACAAGCATAGCGGCTTGAGATGTGTGATCAGCAGAAAGCCTGTTCCGTGGACCTGCTAGAGCGGCT
AAAGCTGCTTGCCCATAGGTTTGCTACTCTGAGCCCTTCTGTGCTGCTTGAGGTTTTTTTGTTTTTGTTTTTGTTTTTT
AAATTTACACGTTGTTGTATAGATGATAAGATGAAGGGGGAAAACAAAACATGTGAAAAAAATGTAAACAGATTTTAAT
GTTCTGTGTGGAATCTGGTTGACAGAGGCCAGGAGTTCCTTTGGGCTAGAGCTGCGATTGGAAACCCATCTGGTCAAGA
ATTCCGTTCCTCTACACAGTAGTTAGTATTCCTGACAGCCTAGGCTGTCGTTAGCTCCAGCTTAGAGATGGAAGAGGAA
GAGAATGGTCTCTGAGCCCAGGGGTCTATGCTGTTGCTGTTGCTGTTAGGATAGCATCTTTCTCTAGGATCTGGGTCTC
CTCCACTAGGCTGTCTAGCCAGCAAGCTCCAGGAATCCTCCCACCTCTGCCATCGCAGTACTGAGGTTTCAAGTGCACA
CCACTACACCCAGTTCTTTGGGGGTTTTCCCACAGGTTCTGTGGTCTGATTGATTGCTATAAGAAACATCTCACAGACC
GAGATATCTCCCAAGTCCTCTCTGAGGTCAGAGAAACTAAAGCAGGCAGCAGAGGACTCAGAGCAGCCCGGGCATACCC
AGAAACACCCAACACTCATGTGGCATCAGAGGAATGTTTCTTTATGTCTGTGGCTTTTCCTTCTTCTCTCCTTCCCTCC
CTCCTTCCCTCCTTCCCTCCCTCCTTTGGGAAGGACTGCTTCTTCTAGACCACAGACAGCTGCCATCTAGAAACACCGG
AAGGCCATCAGCTGATGCCCTGTCAGGCCACTTAAAGCTTTTTATCATGCTAGCCAGCTCCAGTCTGAATTATCCCAGT
GAAATCACATCCTAGTGGCCATGAGGGCTCATGAAGGGAAAGTCCTCCCTCCCTGAGAGCTGTGTTCACAAAGTCAGAG
CTGGGTGAGAGCAGGAGCAGCCATTACCTGGGGGTCCGTGCGTCAGACCTGCCTGGCAGCTTCAGGGCCAGCCAGTGCT
TCTGTGGTCTAATGCCAGTTAGCTATAGCTCCAATGTCTAGACTAGGAAAAAGAACGAGTCAAGAACATGACAATGAAA
ACTGACCAAACAAAACTGTGTCCCAGGTGTCCTTGACCCTTCCCCATTTATCCTGAGTTCTCAGCAGTCCCTCACCCTG
AGTGCTTATGGTACAGTTAGTTGTCCTCCGCATGCCACTGAAACTTGCCTTGGGCCCCAGACCTCTCTCTATCATCATG
TGCATCCTCTCAGCCTTCCTTTGGTCTTTTTTAAAAAATATTTTTATTAGGTATTTTCCTCATTTACATTTCCAATGCT
ATCCCAAAAGTCCCCCATACCCTCCTCCCCTTCCTTTGGTCTTTAAAGGTCACTTCAATTTCTCTGCTTTTTTTCTCCT
TTACTCTCCCTGCCACGCCATCCTCATTTGTTAGGACTGAGGAATGGAGCTGTGAGCATGACCTGCACATTTGGATGTC
TGAGAGTCTCCCTCATATCCAACCCTGCAAGAAAGGTCCCCAAAAGGGAAGGCCTTTGCTAACTTCCTCTGTGAGATGT
ACCTGTAAGAGTCTGTCTCTCTGTCTCTGTCTCTGTCTCTGTCTCTGTCTCTGTCTCTGTCTCTCTCTCTCTCTCTCTC
TCTCTCTCTCTCCCTCCTTCCCTCCCTTTTTGGAATAGCATGCACACACAAATCTAAATATTTACACGGGTTTTGAACT
TTTCAGCATAGAGCCCTTGAGTAAACAAACACTCTCACAATGTTGTCAATGCTCATACTTTAAGACAACAACAAAACCA
GAAAACTTTGCGGAATGCCCTTCAAAGGGTCAGGATATTATTTTGAAAACCTTCATGCCTCTGACTCTGTCCTCAAGGG
AATTTTCCTTTAGTCAATTTTATTGAGGTCTACTTTGCTTATAATAAAATGCACACACTTTAAGTACAGACTCTACCTG
TATAAATATCACTGCTATAAAAATGTAGACGACATCAACCACATCAGAAAGTCCCTGTATTCATAGTTGCAAATTTTTA
ACTTTTGTGGTTTGGGTTTGGCATTCTCAGGAGCCATTCTGAGTGAGACAAGTAAGCCAGGGGGATAGCTGAACCGTGC
TTACAAATTATAGCCACGTCAAAGGGGAATTTTATACATGACAGAGACTGATCATTGCAATGAACCTGCAGTACCCTGG
CTTTTCTGACGGCTGAAGCCTCTAGATCCCAGGTTATTGTGACATTTTGAAAACACGATGCTCACCTGCTTCCTCTGAG
CCAGATATTGCTTCAAGCCCTGGGGGATAATTTGTTCTAAAACTCTCCAGTCATGTTTTCTCCTGTAAGTCTAGAGCCT
GTTGAGGAAATATCAATAAAGAAGAAATCTTGTGTGTTGTACAATCTATAGAAGCAAGAAGTGCCTGCGAACAGAATGA
GAATAGAGGGAGGAGATGGAAAGTGGCTGACCGTAGTAATCCTCGGACTGCGTCTTTACCTTTCCTTGTCTTTCTGTGA
AGCATTTTGGAGTGTTTTGTTGTTGTTGTTGTTGTTGTTGTTAAATGGAGTTGAGTGGAACACCTAGCTTCTTATAGGT
AAAGGGTGGGCCAGGTTCTGGGGACCTCAAGCAAGTAAAGAACTTGCCTCCCAAGCAGAAGGGCCAGAGTGCAATCTCC
AGCATCCATGTAAAAAACCATGTGTGGTGCTATGTGCTCGTAAGTCCAGTGCTGAGTTGTAGACACAGTGGATCCCTGG
AGTGGATCCCCATGGCCAGCAAGCCTTGCCTAGTCCGCCAGCTCCAGATCCCGGAGAGAGTCGCTGTCTCAAAAAAACA
AGGCAGATGGCTCCTGAGGGGTTGACTTCTGCTCTTCATGTGTTATGTGCATAGACACACACACACACACACACACACA
CACACACACATAGGGGGAATTGGCTTAAACAGAGCTGAGCAGGTCTTTAATGAAGTCTTTGACGTACTAACATAATTGC
AGCTTTTCCTGAGGGAAGGCTGTAGGACGCACCAATCTCCAGATATTTTTCTCCTGTAAATCCCACTGGATTTGGCGTG
TGTGGAGCAAGGAGTCATTTTTACAGCTCCCTGAATGTAGCACATGCAGCTTCCGCTTCTCATGGCACAGAGCTGAAAG
CCACAGAGAAGTTCATGACTAGGGGCAAGGAAGGGAATGAGGTTGGGCAGCTTGTGGGTCCAGCATGGGGCCAAGAATC
CCAAACACAGCCCTTGGGTAATCTTTTTGACTAGGGTGTTACAGCTCCTAGGTACTGTGGCTCAGAGTGACCCCATTGG
TAGCCAAAGATTCCATGCAGAGCCAGGATCCGAACCTAGGCCTGTTTTACTCCCAAACCCAAACCTGCCTACACCAGGA
TGGCTGTCCTAAGTGAAGTATAGAGAGGCTGTAACTCAGCAACATGCCCCCACTTCTCCACAGTTAATCTACATGTCCC
TTCAAGCCACATACTCTTCAGGGGGCATTTACCACTTTTTCCCCCCACTGGCAGGTGATCGTTTTTTCTTCTCCTGCTA
GTTATGAGTGAGTATTTGGTAAGTCAGAGCAGACAAGGACACGGTCTCCTGGTTCTGTCCTCATGAAATGGTATGCAGG
GCCAGATGCCTGTGACCCTGGGAGTGGCCCCTTGCAGCAAGCTCTGCCAACCTACCCCTCCATGGCTTCCTCTTTCCCC
TGCATTTTCTCCTGTTGTCATAGCCGCACATCCCACCTGTCTTTATCTGCTGCATCCCCTGGAGGATGGGGCTCAGACC
CGCATCAGTACCTCTGTCCCAGCATCCATGAGGTACCCAGTCTGGTCACAGCTGCAGTGGCTACTGCTGACGTGGGCAT
TGCCGCAAAGGGACATGGCAAAGAGATATAGCAGGGTCCTCACTGTAGCCCAGGGGTGAAGATGTGGAAATCCAGCACT
TACTGGAAATAGCAAAGGAGTATCTCTCAGGAAAGGCTCTGAGGCAGTTTGTCTCCCACCATCCCCGTCTTCCTCTCAG
GAGACAACAGAGGAAGTTGGTAGGAATTACTCACGTCTGCTAATTCATTACTAATTGAAAGGAAGGGAGATGGGTCTTC
TCTAAGCTCAACGCTCAGAAGCTAAAGTATACTAACTCTGACCTTGGGTCCCCTTTTCACGTCTGGCCTTGCCTCCTTG
GCTTCTGTATTTTATTCCATTTAGAGTTGCTGACCACTCAACTTCTGTGGGAGTCTTTGATCTGGTCCCTTTTCACATA
TCCTGCAAGACGTCCCTGGCATCACCAATACCATCAGCGACACCTTTATCCTATCTTCACTAAAATTCACTGGCTTAGT
TAGCATCACCTGTCAACTTGACATAGCCTAGAATCATCCTCCCCCAGCACCCAAAAAAGAATATTTGTTGAGTAATTGT
CTTTATGGAGTTGATCTGTGGGCATATCTGTGAGGGCTTATCTTGATTGTGAATTAATACAGAGAGCTCACCCCATCAT
GATGGCACCATTCCCCGGGAAGGTGACCCAGTGCTGTGTAAGGAAGCTAGCTCTATAAGAACAAGTCTGAGAAAGCCAG
AGAACAATGCTTCTAATGACTTCTGCTTCAAATCCCTGTTTGAATTTCTGCAATGGCCTCAGTTTCTCCATTTTGGAAA
ACAAACTGGGACAAAAACTCATCTTCAAAGCTGATTTCATCATAATATATTTAAAAATAGGGGTTAGAGAGATGGCTCA
GCGGTTAAGAGCACTGGCTGCTCTTCCAGAGGTCCTGAGTTCAATTCCCAACAACCACCATGATGACTCACAACCATCT
ATAATGGGATCTGATGCCCTCTTCTGGTGTGTCTGAAGATAGCTACAGTGTACTCATATACAAATTACATAACTTTAAA
AATATTAAAAATATTCCAGCCAATGAAAGGAAACACAGAAGACACTTTCTGGGAGGCAGGACTGAAGAGATTTCTCTGG
CCAGCAGGTTCTCTGCTGCTTCTGGTGAGGAGGGAGCTGGTTGGTGGGGCTCAGAGGCAGGCAGCAGTTTGGAGAGCTA
TCTATTTCTCTGAGTACGGTGGCCTTCTTGAATATAGAGGCCATGAGTACAGACGGACATGCAGTGAGATCTGGTCTCT
GTCAAAGAAGCCTCGAGTTGGCCTTGCCAAGGCTCTGTCCAAGCAGCTAGGTCATTCCACAGGCTGGAGGGAGGCCCCA
GCAGCAAGACCAGCTCAGTTTCGGAGGGAAATGACAATGAAGATGGAGACTTAGTTGCTCTTCTCAGACACCGGCCCTT
CAGTCCAGAGAATAACTTTAGCAGAGTATGAAACATAACAGCACCCTACCCTTCTCTGGAGGAGAAACACTACCCAAAG
GAAACCTTCCAAGCACAACTTAAGGATGTGGGTCTTGTGGCTTGGTGCCTGTTCATGCAGCAGCTGAGATCCCAGGGAG
ACACAGCCTGGAGCTGAGGACACCATCAAATTCTTCTTCCATATTGCAAGATGATATCTAACCCTGTCTAGTATGGAAC
TCACATTTGCTCAGTACACACAGGACAAGGCATGTGAGGCCAGCTGGGACACAGTGACAATTAACCTCTGGGCATTTCT
CCAAGGCCTTGAGCATTCATCCAGAGGGGAATATGACCAGTCAGAAATTTTCTTGGTAGGAGGTCAAGATAGCCCTGAT
AAAAGCCCAGTAAGACAGAATGAGGATATTCATTTAAGTAGATGGTAGGAGGACTCTGTCAAACCACCTTCCTAGAGAC
ATAAGAACTTCCTGGGCGGGCTGGAGAGATGGCTCAGTGGGTAAGAGCACCCGACTGCTCTTCCGAAAGTCCGGAGTTC
AAATCCCAGCAACCACATGGTGGCTCACAACCATCTGTAATGAGATCTGACTCTCTCTTCTGGAGCATCTGAAGATAGC
TACAGTGTACTTACATATAATAAATAAATAAATCTTTAAAAAAAAAAAGAACTTCCTGGGCCTTGGAGGATGGAGGTTA
TGAGTGAGGACATGCCAGGAGAAGTGGAGCATCCAAACTGAGGCCCAGATTGAGTAGTGAATGACCTTCAGCCCTCTCA
TGGCACAGCAGGAAGACCAACTAGCAGTTCATGAGAAAATCACGATACGGTTAGGGAGCAACTCAAGGTGGCAAAGGCC
CTTGACACTTGTCTAAGGGGGGGACCTTGCTTGGGGATGGCATTTCCCATGGTTTTAGTGGGCTCTTTGTGACAACAGT
TATAACTCATTAGGTAGAGTGCTTGCCCTGGTCTGCACAAAGCCTTACGTTCAACCCCCAGCACAGCACAGAAACAGAA
TGTGGTAGCACAGGCCTGGGAGCTCAGCACAGAGTAGAGGTAGAAGGGTCAGAAGTTCAAGGTCACCTTCAGCTTCAGA
GAGAGTTTCAACCCAACTTGGGAGACAAGAGACAGGGTCTCAAAAACCAAACCAAACCAACAACAAAACCTGGGAAATC
TGGGAGTGACTAGCACTTTCAGACCATTTGCCATGGGGTGGAATATGTGGTATCTGATAGAGTTTCACCCTCAGAACCA
TCCATGAAGTGTTTCCATGAAGAGACAGTGCCAACTGTCTCAGAGAGCCCTGGATGGAAGGCTGGGCAGGCACCCGGGA
CACTGGGCAGAGACCTCACAAGCCTGTCATCTGTCCCCCAGGTCTTCGTGCTTTGTCATGGCCTCCTACAGCTCTGCCA
GTTGCTCTACAGTGCCTACTTCAAGAGCAGCCTCACCACTATCGAGAAGCGCTTTGGGCTCTCCAGTTCTTCCTCCGGT
CTCATCTCCAGTTTGAATGAGGTGAGTGGGAGGCCCTTTATTTTAAAAGACTTTTAAAATCAGACTTGTGTATTGTATG
TGTGAATGTTTTGCCTGTATATATATATATATATATATATATATATATATATATATATATATGTATGTATGTGTGTATG
CATGGATGCATAGCAGACAGATGTTTACTGCCTGAGGAGATCAGGAGAAGGCATTGGATCTCCTGGAACTGGAGTTACA
GATGGCTGTGAACCACTACGTAGGTGTTGGGATATGAACCTGTGTCTACTGCTAGAGCAGCAAGTGCTATTAAATACTG
ACCATCTCTCCAGCCCTGAGTGGGACGCTCTTGCCAGGTTAGGTCATTGTATGCACTGACCTAATCTCTGAGAGGTACA
TAAGAGGGGCAGGGTTTACCTCCTAGTGCAGAAAACCACCCCTAAATTTTGGTCTTTGATTTACTATTGTCCTCATAGA
CAACGGACGGCAAGGAAACAAACCTTTGCCATTTATGTCACCAAAGCCTGGCCCTGGCCCTTGGATGTATCACAAGTCA
CCCACAGCTCTTGGTGAATCCAGACATGGCCTCTGGATTCTTCTCACACCATGTTCTAGGCTCCGTGACTAAACAAATG
AGATCTAGCATATAGGATAGACCCTCTCTTACAGCAGTCAAGAGGAACCAAAACTATGATTCCCACACCTAAACTTTTA
TCTGCATCATTAAATGCTGGGAATAAAGCTGTGGTCCAGGAACACATGGAGTTTGTAGGCTGCTGTTGCTGCTGGCGGT
GGTGAGTGTGTGTGTGTGTGTGTGTGTGTGTGTGTGTGTGTGTGTGTGTGTGTGGCCAGGGGTGTGTAAAGAGAGTCCA
GCCTGGTGCCAGGTTAAGTGAGGGCATCTGAGTGTGTGGTGAGTACAGACCTTCAGACTGAAGCTCCAATCTCAAAGCC
TTCCTGGCCCCAGGTGATATGAAAGGACACAGAATGCCTATGGTAGTTCAAAGGCAGGTGAGGGGCTAGAACAGAGAAG
CCATGGGTATCTTGGCCTTGGAAAGCAGCAGCTCCTATTATTCACTCTGACATGACATTGAAGGATGAACAGACATGGC
GGGTTGGATCCCTAGATCCCTAGATGTTAACCCCTAGTCTTTAGAGCCCTTTGGTTTTGAGACACAGAAGGGCAAAGTG
TGTTCATAATGGGCTTTATGTCAGATTGCAAATGTTTAACACCAGCGAGTCGGTGTTAAGCTGTTTTAGAGGCACTCCG
GGGGTGAGACCTCAAGTCCTCTCCTTGGCTAGAACAACTGCAGCTTTGAGGTCTAGCAACCCATGGCTTCTGCAGGTGA
GCGCAGGCCCACATGCCCCGACCTTTCTTCCTGATTTGTTTTGGACCCCTCCACCATCTGTCATGCACAGACCCTTTCG
AGTCAGCTGACAGCTGAAATGGTCCAACACTTTAGTCCACATACAAAAAAATAATAGAGATGCTAAAAGGAAAGAAATG
AAACCCAAGAAGCTCAAGAAGAAGCTTTTCCTCTCTCTGCAGAACCCGCTTCAGAATGTTCTAGAACCCTCAGGCTGGA
GGGCTCTCTGCTAAAGCCAGGCTGCCTCCGCATACACCTCACACACCCTCACAGCCTCCTGTGTGTGCCGCAGCCTCTG
TTTGTTGGTCATGCTCTTTGACACATGTCAATGAGCACCAACTTTGGGCCAGGCACTATCCTAGATGCTAGGGAGGCAG
AAAGGAACAGACAGACAGAAAGCTAAGACAAACATGTAGAAATAAATCTCTAGTAGGTAGATGGTAGGCTCCATGAGAA
GGTGGCATGGGAGAGGAAGAACATGCAGGGTCAAAGTGGTGGACACAAAGGGACATCCCTTGTAAGGAGCACCAGGAAC
AGTGTTCCTGATGTCCACACTGAGCAGGACTTGGAAGTGGTATGGTCCTTGTCCTTTGTTTTGAAGAATTTAGCATGGG
GATGGGGTGGGTTAGAGTTATTGAGGCAGAAGTCCATTCTTCCAGAAGATTGTTCTAGGACAGTGTAGACAACCCAAAA
TGGGCCTCCGTGGTTCCGGTTACCTCCTGACTTCATGGATGGAGCTATGACACTTACTGTTTGACTGTCAAGTTCTAGT
TTTCTTTCGGTTGCTCTGATAAAGACCATGAACCAAAAGGAACTTGGAAAAGAGAGTATTTATCTCGCTTACACTTCCA
TCCTAGGTCACCATCACAGGAAGCCAGGGCAGGGACTGAAGCGGGAGCAGAGGCAGAGATCACACAGGAAAGCTGGCTT
CCTCCTTCAGCTTGCTTAGATTGCTTTACAACCCAGGACCGCCTGACCAGGGACGGCATCACCCGCAGTAGGCCAGGCC
CTCCCACATTAATCATTAATCAAGGTAGATGTCCCACAGACTTGCCTACAGGCCAACTGATAGAGATATTTTCTTGATT
GAAGTTTCCTCTGCCCAGATGGCCCGAGTTCGTGTCAAGTTGACAAAAAAAATTAGCCAGCAGAAATTTGACATTGAGA
GTAGTAACATGGTGTCTGGGGCCATGCACATTGCTTGGTTGTTATTCTGACGTTCAGTCCAAAGTCCCTGAGTCCATTA
CTTTGGACCTCATTTAAGTTAGGAGTTTAATTTAAAAATATATGCTACACTAGCAGCCAGCCAGGACTGGAGCTTCCTT
GGACTTTTGCCCCAAAAGACTTTTCTAGAAGGGCTAGGGTAAAAGCTTAGTTGGTAAGGTGCTGATCATGCAACCACAA
GGGCCCAAGTTGTCCCATCCCTAGAACCTACATTTAAAAAACCAGGTGCAGTAGTGGCATGTGTCTATACTTCCAGAGT
TGGGGAGGTAGAGACAAGGGGATCCCTGGAGTCAACCAGCATAGACTATTTGCCTAGCCACTAAGAAACTGTCTGAGAG
AGAGAGAGAGAGAGAGAGAGAGCACAGAAGCACACCCACTTGCTAACACACACACCCTACACACACCCACTTGCTAACA
CACACACCCTACACACACACACACACACACACACACTTAGGTTTGTGTATAGACATCCAATATTTAGATTAGTTCTTTA
TGCTTTCAGATAATTTTTTGGGGTTGGTCTAAAAACCTGAGGTCGGGATGGGTTTCTGATGGCCAAGGGAAAGCCAATG
CCGTGAATCTACAATGTGAGCAAGACCATCTGGGCAAGGCTAGTGTGGCTTTGTCAGGGACTGCACTAAAGCCAGACGG
CCCCTTCACTGTCTGTTCTCCCTAGGTGAGCTATGGTAGGTCTCCTCTGGGCTGGAGGAGGGTGGGACCTGGCTCTCAC
TGCATCCCTTGTCCTCCTCTGCAGATCAGCAACGCTATCCTCATCATCTTCGTCAGCTACTTCGGCAGCCGGGTGAACC
GCCCACGGATGATCGGCATAGGGGGTCTCCTCCTGGCTGCAGGGGCCTTTGTCCTCACCCTCCCACACTTCCTGTCAGA
ACCCTATCAGTACGCGTCGACAACTGCTGGTGAGTGGCTCCGACTCCTGCCTGGAGGAACTGAGGCAGGGTGAGCACGG
TGCAGAGCAGATGGTGGTGCTCCTGGGGTTGGGGGTCTCCGCTCTGAGTCACCTTCTTCCCTCTCCCGCTAGAGACTGT
CCTTGACTTCAGTAGTTCATTTTCTCTGCAGACTACATCTGAAGTGCAGAACCTATTGAAGCATGCAGAAGCATGAAGT
AGCCCAGCTACCTGGTTGTTAAGGATGTGGTTGCTGGCCTTTCTCTGTGAGTCCTTGTGCTTTGTGCCCAAGGCAATCC
AAACTCCACCTGCCTAGAACCAGAACCCACGGAGGCTTAGAGAAAAAGAGGACCAGAGTGTGGGACATGATGAAGTTTG
GGGGGTAGGGGGGGTCCTGTTTGTTGTACTGTCAATTTCGTTTTAACTAGCATATCGCTTGTATGGTCCTTTTGTACGG
TCCACTCGCTGATGCCAGGATATTAGTGGAGTGTAGAAAAAAGTTATAATATGAGGTTTTATGTAACTTCTCCTGGAAA
AATATGAACAAATGTCTGTTCACGCCATATAAGACACTACAACAACCCAAATTCTACTCAACTGTAGTTGGTGAATCAA
TAAATTCATTCAGGTTAATCACAGGAATATAGGTAAGAGATGATAGAAATATGGGTAATTCAAAGGCAGCTACATCTCC
AAAAGGCATACCCATCATGGGTGAAGACTCACAAAAGCTGTATGTTTGGAGCCTCCTGGAAAGGTCTAGGTAACTCTAA
CGAGAGTCTCCTCTTCCCCAGCAACTGATTCCTGCTTTGTATGACCTAGGAGGAAGCTTTGTAAATCTTGTAATTTTCG
TGAACTTCTGAGCCATACAAGTTTCTATAGCTCCCTTCATCTTACGAGTTCCCTACTTCCTCTAACCTGAAGAGAAGGT
TTCAGTTGGGAAGAAATGGCTGCATCGTAGGTCTTGACAGTTGAAGGTACTCAGATACTATTTCATGTTCCAAGTATCT
AAGAAGAGAGAGAAGATGTGGGGGAGGTCTTATCTCCTGACCCTCAGATAGGGGGCTTTTTCTGTGACCCAAACTTCTG
CATTTTATTGGGGTGTCTTGTGGCAGAATGAAGTAGACTTTAGAATTTATGTGGTTAAACATGGTATTGATTAGTACTG
AAGTATCTATGAAAAGGTAACTTTTCTTTAGTCTTAATTTAAAATTATTTAATTAGCATATATTAATTATACATAATAG
GTTTCACTGGGACATTTCCGTACATGTACATGATGTACTTGATCATATTCACGTTCTTTCTCCCACCCTTATTAATCCT
GTTTCTTTTCTACTGTTTCGTGTGTGTGTGTGTGTGTGTGTGTGTGTGTGTGTGTGTGTGTGTGTATTTCATTAGGGTT
GATTAAAGGAGTGTAAGTGAGGGTTTGTTTATAAGAACATGGGCACCTTACCACAGGCTTTTCCACTGAAGAAAACGTT
GCTCTTTTCCCTACCAACCCATCAACTGATTGTAAATTGTTGTTGTTTTAAATTGTTTAGTTTGAACTTTATACAAATT
CTAGACAGCTCTTTGTTGATATAAGCTGTACAAATATTTCCTCCCCGTCTCTAACTCATCATGATATCCTTTTTAACAG
ACTATAAAGCCCTTCCAGAACAGAAAGAGGTTTTAATTTTGACAAAATTCAATTTTTCCTTATTTTTTTCCCTTCCATG
ACTCATTCTTTCAGTACTGCACTTAGGACCTCTGTCTGACCCCAAGAGAGAGATTTTTCTATGCACTTTCATGGGGTGT
CTCCATTTTTTACATTTGCATGTAGACTTACGATCTGAGTTAATCATTTAGTTATAAATGCTCAAGATTTCCCTCTTGT
GTATGAATGTCACTTTTCAGCCCAATTTATTACAAAAACTGTCTTTTCAGCATACCATAATTTTGTACCATTATAGCAG
ATAAATTGTCTTGGGCTAAGGCTGCAGGTCAGTGGTAGAAAGCTGACCCTGAGTTCCACCAACACAGAGAAGAAAGCTG
ACGGTGTCTGTGGGTTCATCTCTGGACTTCCCTTCCCACCCCATGGATCTGGGTGTCTCCTTTGCCAGTACTGCAGGCC
CCTCCCTCCCCTCCCCAGTGTTTTATTACATTTCATATATATATATATATATATATATATACTTTGAGATCTAGTTGTT
TATATTCAAAAGAAAACATTTTTTTCTGGAATTTTGATTGGGACTGCATTAAATCCATAATCAACTGGCACACCTGTTG
TGTGTAATCCTCTCATTGTTAAATAGTTTGGCTCACTGCTGCCCTGTGTTCACCACTGACTGATGACTGAACAGGCTCT
AAGCTGATGAATGCCTTTATCCTCCAGGAGAACATAGTCCCTGGATGCCTCAATGCCCACTGTCTATCACTGCTGCCCA
GTGCTGTGGATACTCCTAGCTTCCCCTCCCCACCCTACTCAGTGCTATTAATATTCACCCACAAGGCTAGAGGTTTCTG
TGTTCCTATTCTTCTGGGGAGGGAGACTTTAACATACTTTGTTGCTACAATGTTCCCCTATTCCTGAGCTAGATCTTTT
GTTGTTACTTTGATGAGTGATAAAATGTCAGAAAAGATCTTCCTTTCGCCCTTGCTCTCTAATTGGTTTAGCTGCAAAC
AGCCATGAAATGAGTAGTTATCCCTGCTCAGAACTTTAAGGAAAGTGCATCTTTGTCTTTTGGCGATTCTTAATGGACC
TTCTCCATCTGTCTCATTATCAGCTCTTTATTTCTAGGCCTCCTGGTTTTAGTCTCTCTAGTTGATCGTTTTCTTCTTT
GCTTTGCTATCATAACATGTTTGAATGTGGGCCAATCTGTATCTGTCTGGGAGTTCAGGTTTGTTGGGGGTTGGGAGTT
AGTTGTTGGGATGTATAACCAAGATTTTTATGTAATTCTAAAATATGTCAGTCAAAAATGCTTCTGGTTTTCTTCATCC
TTCACTCTGGAACTTTTATGTAGATATACACGGCAGTCACCCTCATCTTGTAACCTCACTTTCAGTGGGTTGTCCCTCT
GTACCATCTTTTTAGTGATTTCCTTGAAGTACGTCTCCCCATTCTGTGAGTCCCTCTCTAGCTTTGTAAATCAGAACCA
AGCCCTTCCATAATGTAAAATTTAAGAGCTGTCACTGTTTTGCTAATTTCTAGGAGATAGGATTTGCTTCCAAATTTTT
CAGCCCCAATTTTGTGATTTCTTAATGCTTTTTTACTGACATACATATACATAGGTATGTTTCTTTGATGTCTTCAAGT
ATTAAAATATATTTATGTATGTAGCCTTTTTTGATATTGTTCCAATAGCTCAAGCTTTAAGAATGTGGCCCTGTCCCCT
TCATTTTGTCTGCTTGCTCACGTTCCTTGTGGGTCCTCCCTTTGTATGTTCTATCATGTACCATGGAGCAAACTGTAAT
AAGTTATATGTGGACAGATTGGGAGTGGATCTGGTTTAATATCCGTTTGTGTACCGAGTGTGCACTATCTGCAGGGATC
TAGGGCTCTGACCCAGCTGGAGCAACCAAAGAGCCCCAAGAGTTGCAGTTCTCCGAGAGGGAGTCAAAGAAAGGCAGCT
CTTCCACTGCGGATGCTGCCCAGAGTTGGTGGCAGGGGCAGGATGCAGTGGGCAGCTAGGAGCCAAGGTTGGGGTGGGG
TTTGGGGGTCACAGGACAGGAGCCAAAGTAAGGCAGCTCTTCCACTGCGGATGCTGCCCAGAGTCGGTGGCAGGGGCAG
GATGCAGTGGGCAGCTAGGAGCCAAGGTCGGGGTGGGGTTTGGGGGTCACGGGACAGGAAGTGGCTGAGTTAGCCTTGA
GCAGCTTCCAGCGACAAAAGCTTCTAGAGAGAAAACTCTTCCCTAGAGTGTTACAGTTCCATAAGACAGGCACTCTCAG
ACGATTCGGTGAAATAACTCATGCCCTTTATTCCTTCTGGATAGGATCTTATAATACGGTTTTCGGGGGGGGGGTGGGA
ACTGACAGTGGGTGCTTCCTATTGTCTTGGTCTGAGATGTTAGGGATGCCTCATCTGCATGTGGAAGGACTTCAGGTGC
TGTCCCTGCATGACTGATTGTCACAGTCCTCTCCATGGGGTGTCGTGGAAACCCTGTTCCAGGACAGGAACCTGGCCGG
GAGTAGATTTAAACACCTACAGTTCTCAAGTATGATAATTGCCAGGGTTTCTGAATCCACTCCACCTTACCAGGTTTTC
TGCCAGGTGGCCCCACAGGGCAGCATCATGCTTTACATCCTCGTGTGGACTTAGTGCTTCAGGACCACCGCTGCTTTGG
AGGTACCTTTGTATCAACCTGTTAGCTCAGGATGATACTGCCCTTGTCTTGAGAGAAGTGGGGGGAGGTTATGGCTTGG
TGATGAGCGGCAGTGAGGGCCCCGCACTGCAAATGTTCATCCTTAGTTCGATGCAACAGAGCTGTGTGATGTTGGTTGA
GGATGGGTGAGCCACCCAGGAACGTGCTGAGCAAGTAACTGATTCCTGTTTGCTTGAACATCAGGCTCTTCTAAAAGTA
GATGTAAGGGCCAGAGATGGATCATTTAGAATGGCCGAGGGAGGAAATTCCTCTATGGGTTGTGAGGCCTGTGCTAGAG
ATGGGTAGTTGGTTTGAATGGGCGTGGGGAGGATTATAAGCAGGAGTGGGAGGGGTCTACAAATGAAGTGTCTACAAGG
TTCTGGTACTGTCCCTCCTTCCTCTGGAGCGTCCTAGTCATCTGCATAGGCGTGTCGCAGTCGCAGTCCTTGACTGTTT
AAAGGCTGAGCCTTGCTTCAGGAGAGACAAGGACGGAGACAGACCCAAACCTGTGGGTTCTTCCCTGTCCTTTCTCCCC
TCTATCTGGGGGGGAGGCATGGAAACCCAGCTCTGATGGAGAGTCAGGCCTTGACCTTTTTGATGCATGAAGACGGGGC
TAATGAGATCAGCAAGGGAGCCTGGAGCACTGGGGCCGAAGGGAGGGCACTTTGCCAGACACCACACACCAGGCTGGTC
GGCTCCCCGCGGCCTGGGCCCACAGCCCATGGCATTTGCTTTGCACAATGTAGCCACGGGAGGAAAATTAGCGATGGCC
TCACTCTGCTCCTTATTAGGGGTGGCTATTCAGATTGAGTGGGACTTTACCACAAAGCCCAGATCACTACCAGCCCAGT
CTTAACCCCGATGCTCCTGGCCCAAATCATCTGAGAGACTGTGGAAAGGGCTATTAGAAAGAGCACAGCTTGCCTGACA
GGACCACAGTGTGCACAGGCTAGGAGAAGGGGCTGGGCTGTTGTTGCTCCTGTCTGTGCATGTTCTGATTGCAACGAGG
GTAGCACTTGCTCTGACAGACACTCAAGCAACATCTGGAAGCGTTCAGGGGTAGACTGCCTTCGGGGTCCACCTTTCTA
GGGCCAGGCAATAAGAGGAGGAGGCCCGTGACCCAGGAGTCACTCTGTGTGCTTTGGCTGGGCTATGTACTAGGTGCCG
ATGCAGTGTGGAAGTATGGTGTTAATATGTTCAGATAAAAATAGTCATTAAGTCTCATTTTGTGGAAGGAATTATGATT
TGTGGCTCCACTTCCAATATACTCTTATGGGAACTTCAACTTTGAATTATGTGAGGTCATCAGAGTGGGAGCGACACCC
AAATGTGGGTGTGTCTGAACTGTCTTCTGGAGCCAGGCAGTCCTAGGGCTGAAACTAACCATTGAGACTCTCTAATGGG
AACTACAGCCTTGAGCCATTTCCTCCTTGCTTTGAGTCTTAGCAAGGTCTAAAGAGAAACACTTCCCCATCATAAGGCT
CAGGTGTAGCCCCTCAGAGTCCCCAGTGAGAACACACATTGTGTCCTCTGACAAGTGCTTTGCTGTGGGCTGGGTAGCG
TGGGAGGACCTGCAGGTGATCTCTAGTCTTCATGCCTGGTTCTCCAGCACACCCTCATTTGGGTGTATGGTCCCTCTGG
ATGAGAACTTGGCCTGCTGTTCTGACTAGTTCCCAGAGCACGCTGAATGAAGTTTCATCTGGAGCCTCTTAAAGTCACA
GTGTAGGAAAGGCTCGGTCCTCACTCCCATTGGTAGGCAGAGCATGGACTTTGACAGTCCAATCCCATCTCAGCTGCTC
TCTTGCCAACTCTTCTTAGGGGAGGCACTCCTCTTTTTTTCTGAACCTTACCTGTAATACAGGAGCAACAATACCTTCC
CACTCTGTGTTCCCGGAATAGAAATAGACATGAAGAGCGTGGCTCATGGTGGCTATTGGGGAAACCTACCTCTATTTGC
TTGCCTTGCCGTCGCATTTGAAATCTGTAGTCGATCAGTTTCCGCTCCACTCCTCTCTTCATGAGGATTTGCTGATTTC
TTCCCCCATGGAGCAAAGCGGCCCCAGCAACGACCATGGGAAATGAAGGGAAACTATGATCTTCAGAGGGGGAAGAAAA
CTGAAGGAAGGCTGAATGTTGTGGGAAATCACAAAGACCTTTACAACTAATTGCTAATTTAAAAAAGTCCATGCAGGCA
TTTATGGGTGTTTAGATTTTCTAAATGTCACTGGGGTCCGGAAATGTGCCTTGGCGCCTTTAGAGGCTAGCTACCCAGC
CAAAGCCTCCATCCAAGCCCATGTCTAGAAGATACAGGGAGGCTTTTGTGGATGGGAAACAAGCCAGCGTGAGACCGGT
TCCCATAGGAGAGAAGAGAGGGGACTTATGGGAATCAGTGTTTGGAAGGACTAGGTGTAACCTGGTGAAAGATTCAGTC
TAGTCCTTCCGGAAGTCAGCCTGGAAGACAATGGTTGCTGGGGCAGGAAGTCTCTTGAAGGCTAAGCAGGAAATGAAGG
GAAGCGTTGTATTTTGGAAGCAATCAAGAAAAGAAAAGGAAGAAGCCCTTCCTGGTCCTTGATGTCTGAGGCTGTTGGT
CCCTAGGCTGTGCTCTTCGTGATGTCCCCAGGAAGATGGGCAGAGGGAAAACACACAAAGCGGAGCCTTGGGACCCAGA
GTGACTTCAACCCTTCCTTCCTCGGCTCTCGGCCTCCAGAGCTCAATCCATCTCCTAAGCCTGGAAGGGGCCCAGGCCC
GGGGCTCAGACAGAGTCACTCTGGAACACGAGCTCGGGTCTGTGGTAAACACCCAGCTGTGGCATGAAATGGAGATGCT
AGAAGCTCCGGGAAATGAGGGGCAGGCTCATGGCGTGCAGGTGGCCCACGTGTGCCTGTGCAGGGTATGTGCAGGGCAG
AGCTTCTCTACGGGCCGATGTTGGGTGAGGTAGGGAAGTGGCTGGGCATGCAGAGGAGAGAAAGCTGGTGTGTGGGGGG
ATGGGTACTGGTGGCAGGAGTGTGTAGACGCAGGGGTGAAGGTAGGGCGGTAGGAGGGTTGTGAGGAGGTGGGAGTATC
TGGGAAGGAGGGTGAAGAGAACATACATAAGAAGAAGTGTGTGGGTAGGGATGTGGGGGACAAGCGTGCACGTGATGGG
ACTCGGGTGGGGTGGCAGTTGGCTGGAGAGCTGAGATAGGGTGTTACTAAAGAGTAAAGGGAGCATGCGTGTTGGGGTG
AGTGGGTGGGTAGTCAGTGGTTGCACAGCCTGGCTAATTGGAACTGTTTAATCTGGAAAAAAAGGAAGAAGACAAAAAA
ACAAACAAATACAAGAAAGGAAAATATCAAAAAAAAAAAAAAAAAACCACATACATGCTATTAACTCAACTAAAAGAAA
ATCAGTTCCGAACAGATGAGTAGACCAAAAGCAAATGTGATTTGACGTGGACCTTCTTAAAGTCATGGTATAGGAAAGG
AGAGGTGTAAACCTGAGGGATGATTGTAGGCACCCAGCTGCTGCACTGAGTGGCAGGGAAGGTCACAACTCTGAGGTTG
GTGGGGCAAGCTTCTCCACTGCAGTGGCATGAGAGACAGCCCCTCTCCAGGCACTAGCTCCTGTCCTAGGCTGTCCATG
CTCTGGGTCTTTGAGCATTGTAGTGGGTTTGGTCTGATGCTGCTTGTATTCTAATGCGGAATACTGGTTCCTCAAAACG
TGGTTGCCCTCCGCCCCACGAGCAGATCCTCATGTGTTCAAGGGATGCCATGTGAACCTTCTCCCCAGTTTCACTGGTC
AATAAAAGGCCTGTGATTGGGCAGGGAAGGGAGAAAGGTGAGACTGGAGGTTCAAGAGATAGGGTGGCAGGTAGAAGGG
GGAAGGAAGAGGAGGGATATAAGGGAGGAAGAAGCTGCCATGGATCAGAACCGTGTGGCCAAAAGGAACAGCAAGTAAC
AAGGGCCCTTATAGCTAGGAAATACGTTAGTATAGACCTGCCCAATCTAGGCATATGGCTTATAAATAGAATTTCTGGA
TTGTGTGTCTTTTATAAGGGCTTATTAGGATTGAAATTTTAACTACATAGCATCTGTGTTGTGGGCAAGTCAGGTGCAT
TGAAAGCATCCTCCTCGTCCTCCAGTAGGAGGCTAAATGTGGCTGGCTCCTCTCTACCTTCTGGGCCCTTGGGAACTAC
TTACTATGCAGCCATTTTTCTTGTTCAACAGTGAAGCCAGGTAGTGGGCCTGCTGCAAGTGTCTTTGGAGACAAGCTCC
TTCCTGCCCTCCATTGAGTCACTCTTCCTGGATGGTTGGTAGACTCTGTCTCAGTCCTACCTAGGACGGGGGAAGGTCT
CCCTAAGAGCTGTCATTCAAGGCAGAGAAGAAAGGCTTTGGGCATCCTAGGAAACAGTCACTATCCCACTGTAGGATGG
GGTTTGAAGAAACCGTTAGCCATCACTATAGGAGGTAGTGGACTCCTGAAGGCCCAGGGAGTCAGAAAGGCACCTTCTA
TTCCTCTTCCTGTACTTGGTGAGGCTATTGACTGCCCAGACACCTCCTGTGACTGAAATACTTGACATGGATGACCCAG
GCACACCTAGGCAGTGGGCACCTTTCTCCCACGACCATGCTTGCGCCGTCCATAATCAGAGAAAATGGAAGCTCGGGCA
TTGAAATAGGGAAAATAACACAACAGTCAACTGACCCACATCCCAGGAACTAGCTGTTAGCAAAGGCTTGTGGGTATTC
TGGAATGTTCCAGATATCCCCTTCCTGTCTTCTCCCTAGCCTGGGGTAGAAGAAGTGAGGAGAAGGCTGAGTAGGAAAA
CTCCCTCCTCCACCACACACCCATCCTTCAAGAGCCCTGGTTATGTCTGGGTTGTATTCCATATCTCCAGAAGCTTTGG
TTATAGAAAGCTTTAGTGGCTGAAGTGCTTGAAAACCATATCAGGCCATGGGCAGACATTTTTATTCAAGTACCAGGTA
GCAAATATTTCCAGCTTGCCAACCCCAGAGACTATGAACCTGTTTAGCACTGTCCCAGGAACACCAGCTTCCACAGGAG
TAGAGACCAAAGTGTCTAGGCGTGTGCTGTAAAACTCACGGGCACAAACAGGGCAGGGATCCATCCGATCCTAGCCTCA
TTAGTAGTTTCCCAAGCCCTGTGCTACATGGTTCTCCTGGCCTAGGCTTACCCTATCCCTTAGAGCTGAAACCAAGGCT
ATGAATCCACCAGGCTGCCTTGATGTGCCCAGTCACTCTCTTTCTTAGATATGGTACTATCTCCCATGGGTTCTAGTCC
CTCCTAGAATCATCCAGAAAAAGTCTGTTAGTCCCAGCTACAGGAATAGCCACCCCATAGCTGGGCTGAGCCTGCTCTT
CCACTGCGGGCTCTTCACTGGACCTCAGATAATAAGGTTGCAGGGCTTCGGGGTCTCCTCAGGAAAAAGACAGAAGTTT
CCGCTCTGGATCCGAGGATCCTAAAGCAGGGCTTAAGATGTGGATGTGTGATCAAAATAGCCAGAAACTCAACCCTGAG
GATGGTTTTGTTGTTGTTTGGTTTGGTTTGAGACAGGGTTTCTCTGTGTAGCCCTGGCTGTCTTGGAACTTGCTCTGTA
GATGGGACTGGCCTTGAACTCAGAGATCTACCTACCTCTCTCTGCCTCCCGAATGCCAGGCCTTGCCTTGCCTATTTAC
TGTCTAGCTGAAGCCAAGCCTTGTATTTGATCCCTGTAAGAGTCACTCTCCTTGGTTGGAACCCTGCTTTTTTTCCCTT
GGGTTTAATTTTTTTTTTTTTTACTTTTCGGCCTTCGGGATGTTTTATCGGCCATTCTCCTGACTCTGTTGTTCTTAGG
CCTGCCAGCCATGTCCCCAGGACAGAGCATGAAGAGTGACAGACACATAGCCCTTCTACCTCAGGTATACAGTAAGCTG
GTACCCCTGAAGATGCTCCAGATATGGGTCCCCATCTTACCTAGCAGATATAGTGACAGACTTTGTCAACAGGCTGCCT
CCAGTCAAGTCATGCCACAGCTTCTGCTGGCTAAAGAGCCTCACTAACAAAGAAAATGTGTGTGGGGGAGGAGGCATGG
ATGACTTTTGATGAACTGATGTTGGCTCTGCTAACTACTCTGGGTTTTCTTCAAAGAGCTCCCCGGCAGCCTGGGGAAT
AAATCTGTGAGCAATAACTTCTATTTCACAACAATGAAAGGTGCTAGTGAAGTGCCTATCTCCTTAAATATTAAAGTCG
GTGCTGCTAGCTGTCATCATTTTTCTCCCAACCACTAAAGAAATGAGTCTGATGCTCAGGGTGCCTGTGCACTGGCAAA
GGCTATAAGGAACTAGGAGAGGGGAGTCTCAGGCCAGCCATATGGCTTGGGATTTTAAGTCTTGCATACAGCAAGCCTG
CTCCCTTAGGCCCAGTACAAGTAGTATTATTTTAGAACCCTCCTATGACAAGGGAGGCAGTGTGGGAAGAGAGTTTCAG
TCTCAGTGCACACATACATGTGTGCACATACACAGACGTGCCCAACACTAGCACACTCCTTTCCTGTACATGTACGAAC
TTTTCCAAGAACACACTTTTGCCCTGTGAGTTCTTTGTTGTAATCTGCTATTGCTGGAAACCGCCCTCAGTCTTGCCTG
TCACTACTGGAGCCTGTCCCTGAGGAACATGGCCTTATCCATTTGAGTTTCTCCAAGTGATTCATCATTTTGAATGAAA
GTAGTTTTATTGGAGGAACTTTGGGTCCTCGATTATGATATACTAATTATTTCTTATAAAATAATGCTTTATAAAACAA
AGTTTTATGAATAAGGAGAGCACAAAGTTGTGAAGGAGTGGGAAGGTGGGGATGGACGTGGGAAGAGCTGGGGGGATGG
GAGGAGAGTGACTATAATAAAAAATACATTGTATAAAGTTCCCATAGGATCGTAAAATAGTATATTAACTGTTTTTCAT
TGCAATGACACATAGTGATCACATAAGCATAGCATGTGACATTTCAACCTATGTACACATGTGTGCTGATCTGATTGGA
ATGGCTGACATACCTTCTATCATGCTAGCCACTTACCATTTCTTTATGTTGGGAACATCATCCCTGCTTCAGCCCTGTC
TCATGCCTGCCTGAGTCCATGGGTCACATCTCAGGAAAGTTGACTTCTTGACACAAAATCTCCTATATTGCAAAACCAC
AGAGAGATCCAGAAGTCAGGACTGGAATTTTGCAGATATAAAACTCAGCGTGAGTCAGGAGGTTGACTCGTGTGCTGCC
AGGGTGGGGCTGAGTAACCTCAGGAGGGGTGAAGCCAGCCTGTTCCTAGTTAGCACTGCCCAGTCCTGTAAAGACTAAT
GGAACCGGACCCCTTGTGTCCTACTGGGGTTAAAGAATGTGCTTTGATGAACTTGTGCCTTCCAGGAGTCTGCCTTGGA
GGAGATAGGAAATAGACTAAGCTCCTGTTAATAAAAAAAGTTTCTTTTCCTCCTAAGGAGCCCTGCAGTATAGAGGCAG
GCAGCCTTGGGTGAGCAAATGGATTTCAGAGAAGTTCTGTAGTGGCTGCAAAGGGTGCCACTTATCTGTCTTATTTCAG
GACTTTTATTATTTTTAAAACGTTGTGTGCGGGGGGGGGGGGGGGGGAAGGGGACTGGAGATATAGAGATGCACACCTT
TGATCCCAGCACTCCGGAGACGGATCTCTGTGAGTCTGAGGTCAGCCTGGTCCACAGAGTGAGTTCTAGGACAGCCAGA
GCTACAGAGTGAGTCCCTATCTCAAACAAACAAACAAATACACCAACAAAAATGTAGGACTGGAGAGATGGCTCAGTGA
TTAAGAGGACTTACTCTTGCAAGGATCCAGGTTTGGTTCCCAAAGCCCACATGGTGGCTCATAACCATCCATAACTCCA
GTTTCAGGGAAGTCAATGTCGTCTTCTGATCCCTGAGGATACCGCGTGCACATACATGCAGGCAGAACACTCATACATA
TGAGATAAATAAATCTATAAAAAAATTTAATTTGTATGTGAGGTAACGTGTATGAATGTGTTTGGATGTATGTTCATAT
GTGCACATATGTCCATGCACACATTTATGTGGAGGCTAGAAGTCAATGTTAGATGATTCTCAGTCATTCTTAGCTGTAT
TTCTTTGAGACAGCATCTCCCACTGAACCTGGCGCTCGCCTGTTTATCTAGGCTGTAGGCCGTGAGCGCCAGGAATTGG
CCCTATCTCTGCCTCCCCAGTGCTGGGATTACAGGTGCACGCTGCCACGCCGGAGCTTCTCTCTGTGTGCTGAGGAGGA
TCTGAAGTCAGGTCCCCATGTTTGCTCGGCCATCACTTCACTAAGTGAGCCATGTCCCCAGCCACAGTGAGCAGCCGGG
AAAGCGCTCAGACTTGAGAAATGAGATACTTGTTGATCCCTTACACGGGCTCAAGCTCTTGGAAAAAGCTAAGAACTTT
GCCCCTAGGGAAGAAATGGGAGACTGCAGCCCAGAAAGCCAATCTGTCCTTCTGTCTGTTGAGAACCTGCCTGCAAGGA
TGGTCCCCTCCAAGGCCTGTTCTTCCCATCTTCCTGCCTCTGCAGTCACATATCTATGATGCCCACCTCATTCATGCTG
ATTATTTTTGAAAAGAGCTTAACGGTCTTTAAAAAAAAAAAAAAACTAAGACCCTATTTTCACCCAGGGTGTGGTGACC
TAGACACCCACTAAAGGGGGTCCATGCCTTGTTCCTAAGCTCCTGCATAAGGCTCCAACTGCTGTCAACCGCCCTGGAG
GGCAGGGAGCAGCAGACCTCCTCAGAACATCGTGCCCACTGCTCTTCTCTGTTCCTCGGTGCTCCAGGGTCAGTGCTCA
CCACCTCCAAGGTAGCCGACTGCTGTTCCTCTGAGATGGCACCCATAGGGAAAGAAGCCCTGCCCTAAGGGCTTTGTTT
AACTACTTTTGCTCATACATGTGTGTGTGCATGTACATGTATGCATTCATACATGCATGTGGGGGGGTGGGGGGGAATA
CATCCGGAGGCCAGAGGTCATCCTGGGTGTTGTTGCTCGCTCAGGAGGGTCATCCACCATATTTTTCGAGACATATTCT
CTCACTGGGACTTGGGCTGGCCAATGAGTCTAGGCTGGGTGGCCACATGCCATGTGATCCTCACCTTTCCAACACTGGC
GTTACAAGCGTGTGCCCTCATGCTGGGCTTTTTATATTGGTTCCAAAGATCATATTCAGATGTCATGCTTATGAGGGCA
AGCACTTTATGAACTCAGCCATCTTCTCAGCTCCAACTACTGTCCCTTTGCACCAGTGTCACAGCTGGATCTGAAGTAG
GATTGGGCCCCCATTCAGGACTGGCATCGGGAAGGGCATGTTCTGCAGGGAGATCTTGAATCAATGTGGGAAACAGAAT
AAAGCCATGGAGAATTTGGAGACTTGGATGTAGTTGGGCCAACTGATTTTTTTATGCATATTTCGAGTAAGATGTATTT
CTGACCTGCATATTGACACTTTTAAACTTAATAGTAGTGAAACGATCCCCAGAGACTTTGGTGGCTGAACACTTCGCTG
GATGAAACTTCAGGAGGATTTCTGTGGCTTTGGCATACCACGCAATATGGCACATGAAGACTTAATAAGGCAGTCAATG
TTTTTACGATCTTTGATGACCATTAAATCTCTCTCTCTCTCTCTCTCTCTCTCTCTCTCTCTCTCTCTCTCTCTCTCTC
TCTCACACACACACACACACACACACACACACACACACACAGAGCCTTTGACAGCCAAAGCAGTAACGATGGTTCTCAT
GAATGTCTGTTTCTGTCAAATAATGAGGACGGATGAAATCTGGAATTAGAGTAAACTTAGAACAAACTCTATTCTCTGG
GACTTTTATTTCCCCGGAGAGTCCCTCAAGTCGTCATCCCCTGCCCCCTGCTCCAGTTCACTCCATTCTCCCACCACAG
TCCTACCTATTCATACTCATACGACCATGGAGCCCCCGCAAGCCCTGACCCTGAGCACACAGCGACAGCCTCACCTCTT
AGAGCTCTAGGCAGTTCTGGCTGAGGATCTTCCTGCCCTTGAACTAGGGCCTTAGTCTACCCAGAAGCTGTATTACAGT
GCCCTGTCAGGGTCCTCACCTGTCTTGGGGTGTCCTTGGGGTACTTCCTAAGGCAATCTACCCACCTGCAGTAGCTAGA
TTGCATACCCCCCTCCCAAACTTCCCTGTGAACTGTGTCCCTCACTTGTGAGTGCTGCGACACCCTGGCTTCCCATTGT
GCCCTCTTCTTGGTTCAGAGGCCCCTGGTCTCCAGAGAACTTTTTCCAACACCTCTCCATCCCCCTGCCATGCCAACTC
TGAGTTTCCCATTTAACCTTTACTCTCTGTCCTTTGTGTGCTTGGCACTATTTTTTTTTTCCAGTTACGGTGAAATGTG
TAACGTGAAGTTTGCCCTTCTACCACTGTAAGATGCGTGCTGGTGGGGCTAAGTCAAAGCCACCCGCCTCCAGGACATT
CTTATGTGAATCGATCCTATGCAATGTCTTCATTCGTTTCTCCCTTAGCCCTTGACAAACTCCCTGTCTTTATGATTTC
AGCCATTTTAGAAGCCTCCCATTAAGGGAGTCAGGCAGGACTTGGCATTTTGTTTGGCTTCTTTCACTTAGCACAGTGT
CCCCAAGGCTCATATATGTTACCATACAGCAGAATTTGCTTCCTCTTTAAATTGAAAAAAGATCCTATTGTATGTACCA
TGTTTGACTTAATTACCAGGTTGTTTCTATTTCTTGGTTTTCACCAACAATGGTGCCATAAATATGGGAACATTAAGAT
CTCTTCAGGGCTGAAGAGATTGCTCAATGGTCAAGAGCACTGACTGCTCTGCCAGAGGTCCTGAGTTCACGTCCCAGAA
ACTACACAGTGGCTCACAACCATCTGTAATGGGATCCGATGCCCTCTTCTGCTGTGTCTGAAGACAGCTACAGTGTACT
TATACACATAAAATAAATAAATCTTTTTTTAAAGCGGGGAGGTGGATCTAAAAGAGGAAGAAAAAGAAAAATCCAGCAA
GGGCAGTGGCCGTGCACACCTTTGATCCCAGTAGTTGGGAGGCAGAGGCAGGCAGAGGCAGGAGGATCTCTGTGAGTCA
GGACAGCCAGGGCTATACAAAGAAACCGTCTTGAAAAACAAAAGCCTCTTCAAGCCTCAGCTTCATCTCGATTATTACG
GGACTCATTGCAGAGTGGCTGCAGCCTTTCCAACGCTTCGACAGCAGTGCCCAGGGGTCTCTCCGTCTCAGGATCCTCA
CCAACACTCGCTGTTTTCCGGCTTTTTGATGCTTGCCATCCTGTGGGACATTACATGCTATCTCATCGCTTCCTGTTTC
TGTTGGCAAAGCAATACTGTAGTTGCTTTTTTCTTTTTTTTAAATAACCCAGGGGCCCTTGTGGGTTGGAGACTAGGTA
AGGGCCATGCCTTTGTCCCTCTGTCACCTGCATGAGGAAAAGGCCTTCTCTCACAGTCACTAGCCAGATGGCTGGGAGA
CTAAAGAGGATTTGAATCTTGGTGTGACTGTTTTTCAAGAGGCAGGCTTCACCCACACTGCTAGTCAGAGGATGGTGTC
TCAGATAGAGGCACATGCCTTCCGTGACCAAGCAGGCACAGGGACAACTCTTTCTTGGCATTATCATTTGATTTCAGAT
GGCCTTTAAAATATTCTTGAATAGATAATGCACACCACAGGACACTCACTAAAGGCGACCAAGGCCCCTGTCTCCGTTA
AAGTCATCTGCATTCTGTGCCACCAATGAGCCTTGGCTAGTGGTTGTAGTAACAGTTTGGTTTGTTGAGAGGGGTACAT
GTTATGTGGACCAGGCTGTCCTCTAACTGGTAATCCTTCTGCCTCAGCTCCTTGAGGGCTGAGATCCCTAGTGAGCCCG
CCATGCATGGCTTCCCAGAGTACTTGATAGTATCACACCTGGCCTTCGTCCCTCTGTTTTATCTGAGTCTCTCAGTGGC
CATTAAAGGAGCCCTTTTGCTGGAAGTTTGCACATGGGTCTAGTCTAGACCCTGGCATTGAGAACAGGAAGTGATGGGA
TTCGGCCATAGGAGGCCTGTGTGGACGGGGAATGGGGCATCAGGGAAGGCGGAGCCCCTGCTCCTGCCCTACCAACTAT
TCCATGGCCCCAACCTTTTCTCTCCAGGGGGCTCAGGGGCCCTCTCACTCAGGGCCACATCTCTAGGAAGATCCTAAGT
AGGAATTAAACTGATTCCATTCGTGTTAATGAAAACCCCACAGAGGACGGCTGGTGACGTCAGAGGCTTCCATTGGCCA
GAATGAAGCAGGAATAAAAGGAATCTGGAAAAGATTCCCTCAAACCCCTCTAGGCCTTGGAAAGCTGACAACGCTGATT
GTTTGGTGTGGGGCAAATTGAAAACAGAGGCACAGAAGCAGCTGCCAAGCCTGGCAAAGCCTACAGCCGCAGGAACCAC
AGTGTCCAAAGGCCATACTGGGATGACTGGAGCCCTCCCAGAAGGGCCTCCAGGGGCAGCCTCGCTCCCCAATCTGTAA
TATGACAGGGTCTCCAAATCCCCTTGTTTTCAGAGTCTGCCGTTCTTCTCTTGGTCCCGGCCAAAAGGCCCAAGAAGCA
TTAAGAGTCTATGATTCTGGGGGGCTGCCCAGGTGTGGTCAGGTCCCTAAATACCTTTGGCAGCCAGTCCTTCGCCATG
GTGTGGTGCTGGGAGCATTCAGAAGTGAAGGATCAGGGGTCAGGATGCAGGGAAGGCCAGAGCCAAGCCCAGCGGTGAC
ATAGTCCTTTGCTGAGGAGAACTCTGGGTTTAGCCTGTGGATCTCCTCTTCCAGATGGGTGGCGCTTTCCTCCAGGACT
GAAATACGGATCCACTATGCTCAACCCTCTGCCAGCACCAGAACAGAGGGAGAAATGGTTGTGCCTTCTCCTTCCTGGG
CAGAGCTGCTCCTGGTGCAAGAACCGCCATCCTCCTTGCTCTCGCCTCAGTCTCTGTAGCAAGCAAGTCTTCGGCCAGG
CAATCAGAGGACTACTCCTCTAGGACCATGTCCCATTTTCCTAACCCTGGCCCTCTTTCCCTTCCTCTGGGATATGGAA
GAAAGGCTAGAACAAGAGCGGGATAGAAGGACAGATGGTGGAGGCGGGCAGCCAGGCTGATGGTGACTACTGGGCATGC
TCATTGCAGGAAACAGCAGCCACTTTCAGACCGACCTCTGTCAGAAGCATTTGCCAGGCCTGCTCCCCAGTAAGTGCCA
CAGCACCGTGCCCGATACCCAGAAGGAGACCAGCAGCATGTGGAGCCTGATGGTGGTCGCTCAGCTGCTGGCCGGCGTT
GGGACGGTGCCCATTCAGCCATTTGGGATCTCCTATGTGGACGACTTTGCAGAGCCTACCAACTCGCCTCTGTATATCT
GTGAGTCTTAAGCTTCAGCCGGGCTGCGAGGGGTGGGGATCAGGACCTGGTGCAGCACCTGCTAAAGCTCTTGGGGATG
AAGTGGGGTCTGATTCACTTGGCCCACAACATGGTGGATGACAAAGACCCATTGTTAATGCCAGTTCTGCTTCCTTCCA
CTTGGCTGGACCTCAAGTGGCTAACCCAAGTCCCCAGGCTTTTACTTAGAGGACGGCACTAAGATTTTTTGTGTGAGCC
TTGTGTTTTGAGGTTAGGTGAGCTGATGTAGATCACCATTTAGCCCTGTTTTGAGCCTTTGACATCACAGCATGATATT
GGATTCTATCAAGTTCACACTAGAAAACCATGCAATTGAGGTTTTTTTAAAAAATGCAACAACAACAACAACAACAACA
GCATAATGCTTTAAGTTTAAATTTGGTATTGGACCACGTTCACAACTGTCCTGGAGACCATGTGGCCTATGGGCCAGGT
GTTAGACACTCTGTAGATCCTCAACAAATTATAGGCTCTTCTAGGTGATATACCAATACTCCGAGTTGTGGACAGGTTA
GCATGCATGAAGAAGGTAGCAGATGGCCAACTGGTACCTAATCCCTTCCCTTAGAGAGAAAGCCACATCGAGCTTTCTC
AGGGTCACAGCCTGTACCTTTGACTCCACGTGCACCCGTCAGAGCCACCAGCACCGTGCACACACAACACTGTAACTAT
AACACAGAATCCGCGTGATGCTGTACCCATCCTGGATTTAAACTAATGGAGAGTAAATAAATAAAAGTAGATTTGTGCT
CTTGTAAAAAAAAAAAAAAAAAAAGATACCATTGAGGAAAGTTCATTCCAGGCCAGCAGATCTCTCTGGCAGGTGCTGG
AGGAGCCGCAGGCTTGGTTCTGCAAGCCTTGGCATCCGGCCCTTCCTTATCTGCAGGAGCTGTAATAGGCTTGGGCTTC
AGGAGGAGTTACAGGCATGGGCTTGTTGGTCCCAGGGGACAGCGAGTGGCACCTTAGGAATCTCCTTCAGTTTGGGAGA
CTAGGGATGCTGAACTCCCCCCAATGCCCCTCACTCGGCTTTTTATCCCTTGCTTTAATTTCCAGCCATCTTATTTGCT
ATCGCCGTGTTTGGGCCGGCTTTCGGGTACCTGCTGGGCTCAGTCATGCTGAGGATCTTCGTGGACTACGGCAGAGTGG
ACACGGGTGCGTATGCAGAGGACACTCATTCTTACCTATCACCCCTTTACTGATGCTTCTGGTTTAGCTCCTCCCTTGT
GGCTGCCCATAGAGAGCAGCGATGGAGCTCTCAGCCCATGTGGGGCCCTGCCCTGAGGTGCCTGTCACTGAGTGACTGG
ACACTATTTTGCTACTTTCTTCCTCCTTCTGTGTCCATGTCTGATGTCAAGGAACACAGAGGGGGCTGGAGTGCAGTGG
GACAGATTTTCAGGGGATCATTGCTGTTTGTAGAACAGGCAACCCACTTTGTTGATGGCGATGTCCTCCGGCTCCTAAG
AATTATGGGACCTCCTTTCTTAGGGCTCCTCAAAAGTAAAGTGTAGCTGGGCGTGGCAGCGCTCGCCTTTAATCCCAGC
ACTCGGGAAGCAGAGGCAGGCAGATTTTTGAGTTCGAGGCCAGCTTGGTCTACAAAGTGAGTTCCAGGACAGCTAGGGC
TCTCCAGAGAAACCTGTCTCAAACAAACAAAACAACCAAACAACAACAGTAAAGTGTTCTCAAGCTTAGTGGTTACGAG
TGCAGGTGAGGATGGCAGGCACACAGGTTTGGAATTGACTAATGGACAAGAGATTTGGTCTCCTGATCAGTTAGAGGAA
GTCAGATGTGTGTAAGTCTTCCGTTACTGGGATGCTAGAAGAGCCTGTAGAATGGTGGCGGCAAAGTGTGTAGTAGTGT
ATGAACCATGTCAGCCCTCTGGAGACCAGATCATTTCACCAATTATTCCTGCCAAGAGCTAGAGAGGAAGCACAGCAGT
GGGAGGGTTAGCATTTGCCTTCTGTGCAGAGGCTGGGGTGCTCTGGGAAGACCAGAAAGGCCTGGAGAGGGAGAGCTGT
CCCACCACTGTGGGAAGAAAGTCAGTAAAACACCCCCCTGAACCAGAGTTCCCTTTCGAGTCAGGTCAGAGAGATCGGG
CATTCAGCCAACAGACACTTCTATCCCTCGGTTTCTTCTCAGCTACAGTGAACCTGAGCCCAGGTGACCCTCGGTGGAT
CGGAGCCTGGTGGCTGGGCCTGCTCATCTCCTCAGGCTTCTTGATCGTCACCTCTTTGCCCTTTTTCTTCTTTCCCCGA
GCGATGTCCAGAGGAGCAGAGGTAAGTGTCCCAGGGCCCAGGACTCCATGGATGTAGCCACGTAAGCAATGGACACGGG
CTTGGAAAAGAAACAGCTGGGAAGCTGCAGTGGCCTTGGCAAACTGGGGGAAATGAAAAGAGGTCTTAGGACTTAGGGT
TCATCAGAGGCTGAGAAGAGGTACCCCTCAGCACCTTCTTATCAGGCTGTCATCTCGAAGACCGGAGGGTTGCAAAGGA
ACATAGTTTGGAAATCCCTGAGTTCATCTAAGCTGGGCTAAAGAGTTAAGAAGCAATTCACAGTGCTCAATGGGATGTA
GAGACAGCGAGACAACTCAATCCTTAGAAAGCCGGCCATGCAAACGTGAGGCCCTGAGCTTAAGCCACACACCGCCTCT
TTTTTTTAAAAAAAAAAAAAAGCCAAGCGTGGTGGTATATAATTGCAATCCCAGAGCTGGGGGGGGGGGGGGTCAGATA
AATGGAAGTATTCCCTAAGGCTTGATAGCCAGACAGCCTAGGTGGGGAGTTCCAGGCTACTTAAAGACCTCTTTAAAAA
TCAATATGGGGGCTGGAGAGATGGCTCAGCGGTTAAGAGCACTGACTGCTCTTCCAGAGGTCCTGAGTTCAACCACAGC
AACCACATGGTGGCTCACAACCATCTGTAATGGGATCTGATGCCCTCTTCTGGTGCGTCTGAAGACAGCTATAACTATA
TATTCTTTAAAAAAAAAAAATCAAGATGTGTGGTGCCAGAAGGATGACACCCAAGGAGGTTGGCCCATGACCTCCACGT
GGACGCACACATGCATGTACACATGTGGACACACACACACACACAAAAGCTGAGCAGGTAGTGAGGCACAGTTAACTTG
GACATTTAGTGGAGTTTACTAAGGTTTGAGCCTGAATGCCTGGTGAGAAGGGGGCGTCCACTCAAGGTGTAAGTGAGGG
TGTGGGGCAAGGATGGGCTGAGAAGGAGCGTGTCTGGGACAGGGTTGTATCTGCTAGGGAAAGGAACCTGAAAGATGAG
ACTAGGAAGCAGCAGAGAGGAGGTTGAGCGGAGCCTTTATGTTACACTAAAGGGTCTCACGTTTTCTCCTGTGGTTTCT
GGGCACTGCAGTGACTGTGTGGTCCCATAGTGACAATATGGCAGTTGGCTGAGATTGGGGCTCATGAAAGAATGAAGAT
TGGAGACCTAAGAGGCTCAAGAGAAAGAAGGAAAACACAGTTGAGGGTCAGGGCAGTGGCCATCAGAGAGATGGGACAA
AGTCCTGCCTGGAGCGGTAGTGCACAGACTTGGCTGTGGATAGGCGTTGGAGCTGCTGTTTTGTGTCTATGACTTAAGA
CAGAAAGGGCCCCACTGTGGGAAATGGAGCTGTGAGCAGAGGTCCAAAGCTTATCCCGGGGAAGCATTTAAGAGGTTAT
GGGTGTATTTTGATAGGCTAAGAGTTGGACAGCTCTGAGCTTGAAGACTGTTCCACTCGTAAACTTGTTGAATGATGCT
AGGCAAGTTGCTTTGTTTTTAAGCTTCCTTTCTTGTAAAGTTCTTGGGAGTAATTAAAGAATCTGTCTGGGAAGGAAGT
TGTGAGGGAAGAGGCGATGGATGGAGAGTAATGGGCATGTTGCCCAGAGCAGGAGGGCTCAGTTAATGGGAGCATCAAA
AAATGCTTATGGTAAAGCCCTGGCCTCTGTCTATGCAGTGGCTTGGAGAGAGCCGAGGGCTCTGACTGGAGCTGCTAGC
TTCAGAGACTGGCGAGGGCACTGAGGTAATGTCCCCAGAATGATTAGAACAGAACCTAGGGTAGGGGTTTCTTCTGGAC
TGAGTAGAAAGCAGGGTCCCAGAGAGGACACAGAAAGGGGGAGCTGAGAACTGCAGACATCGTAGATCCCTGTGTCCTA
GAAGCTAGCCATCTCCAGGATCTGCATATGGGACACAGTGTAGTCATAACGCAGACAAGGGTAGGGTCCAGGATGATGC
TTTGGGCATCAGTCATGGGAAAGTCAGATGAAATAATATGCCCATAGGTATAGTGGGAGGCATTGGCACAGATTCACCC
TGGCCTCCAGCCAAGGCACCAGGCTCCTGAAAAACACAGGTGGTATCTGCTTCAAGGGAGCATCCTGCAGATGCCCTCC
CCCACACACGTGTGGTCCTCACGCCGCTGAGTCAAGGGGCTCAAGGATGGGCCATTAACATCACCATGTTTGCTACCTT
CAGAGGTCTGTTATCGCAGAGGAGACCATGAAGATGGAGGAAGACAAGTCAAGAGGCTCCCTGATGGATTTCATTAAAC
GTAACTGAGCGCCCCAGCAGCCTCGGGTGGGCTGGTTCTGGGAGGCAGCAAGCCAGGTACAGGGTAAAGGGCAACAAGA
GACAATAAGTAAGGCCCGTGACTGCCTCACCTCCCTTCAGTTCGGAGGGATCCTGCCAAGGACCCTGGTGACCGGTTCT
TTACAGGAACCAGTAAATCCCACTTCAGGCAGAGCCAGTCACCCCCTCTCAAGTCTCCCCTTCTGCTCTCTCTCTCTCT
GTCTTCCTATAGTGGTGGAAGTTGTGGGAGGAGACCTTCCCCAGGGTGAGCAGTGTTTCTATTTACCCACCACCAGCAT
CCTCTTAGTCAGACCACAAGCGTGGTGAGCAGGAATCCAAGGACCCTGGGCTGACTTCTGGGAGTCTGGGTGTCTCTGA
TTCTGCCCCGAGGAAGAGAGGAAGCTGTCCTTGTGGATGAGTCAGGGTGACGGCAAGGCTCTAGGAAGCCTGTGATGCT
CTTGCCAAGGCCTGCCTCCTGACAGCTGTTTGCCCGCAGGGTTCCCCCGCATCTTCCTGAGGCTGCTGATGAACCCACT
CTTCATGCTGGTGGTCCTGAGCCAGTGCACCTTCTCCTCAGTCATTGCTGGCCTCTCCACGTTCCTCAACAAGTTCCTG
GAGAAGCAGTACGATGCCTCGGCAGCCTATGCCAACTTACTCATTGGTGTGTCAGAGGGCACTGCAGGGCCTGGGTGGT
GCCCTAGGGAGGAACTGGGTACTGTACAGTCCATCCAGAGATAACTGAGAGCCAGACCGGATGCAGGCAGGAGACAGGA
GGGCTTGCCGGAGAGAAAGAATTTTAAGAGCATTTTTCCTGGTTGGCGATCTAGAGTGATTTTGGCAGGATTCTGAGAC
TCCTATTCTATTTAGGAGTGGGTGAAGACTAGGGGCTGCTTTCCTTCCCATCAAGAGGGTAGGAATCTGATATCCTCCC
AACTGACCTTATCAGGGGTAACAGAGGAGAGAGGGAACATGGAGCTTGAGATATACATAACACCTCTTAGGAGTTGCCT
TCTCCAGGAACTTGAGTCTTGGCCCTGTGACACGTCAGCCTCCTTTACACGATGGCAGTGCAGATGCTTAGCCCCCACC
CACCCAGACTAATGAACCCAGATTCTGTGGCAGGGGTGCTCGTACTTCTTGCAGTAGATGCTAAGAACCACTAATGGGG
GTTCTACCAAGTGCCTAAATGAATAACTGGGCCCCATCTATAGGTGAATTTGACCAAAGATTTGACAAGATAATCAGGG
CATGAGATCTGGGGTGACTTCCCTGTTTGCGCTGAAGTCCTGTGTAGCCTTTAGGGATGCTATCATTTCTCCAGCCTTC
AGTGTCGGATCAGGGCATCGCCACCCAAGAACGATCTACTTCCTTATTGTTGTCATTGTCTTCTTGTTTTCAATTTGTT
TCTTCGAAACAAGTCCTAGAATAGCCCAGGCTGACTCTGAACTTGCTAGGTAGCTGAGGATCACCTGGAATTCCTGATC
CCCCTGTCTCCACTTCCCAAGCGCTGGGTTATAGGTGTCAGAAGTTGACTGCCTCTTTGCTTTTTGATCAGGCTAGCCA
ACCCTCTACTAAAGTAAATTCTCGGGATGTACCCAGCAATCACCCAACAGTTAGTGATATTCGGTAGTCACCCATCGTT
TCTTTACGAAGCTGTGCTCCTTCCCTGTGGTTCAGCTCAAGGCTCCCAGGCTGTGCATCCAGCCCCTCTCCTTCTCACC
TAGGTGCTGTGAACCTTCCAGCTGCGGCCTTGGGGATGCTGTTTGGAGGAATCCTCATGAAGCGTTTTGTTTTCCCTCT
GCAAACCATCCCCCGAGTAGCTGCCACCATCATGACCATCTCCATAATCCTATGTGCCCCTCTCTTCTTTATGGGATGT
TCCACACCCGCCGTGGCTGAGGTCTACCCTCCCAGGTAATGGACAGCCGGAATCCAGCCACTGTATGTCTCTCTTCTCT
GCCTTTTACCTCCTCCGTCCTGCTCATTTGATCTGATGTCTCACATGGTGACACAGGAAGCCAGGTCTCAAATAGTGTG
TTCTATTATGAGATGAGATTAGCCAGCCAGTGGGGTTGGGCTTAGGGTTTGGTTTTGTAAGAAGCCACAGAAGCTAGCC
GGCTTAGTGTCTGGATGAGCCTAAGCCATAGAAGCCAGGAAATAAGCAATGGTTAATGCCAGCTCTGAACCAGCTAGAG
CTAGAGTCTCACTGAGATGTCGAAGAAAGAGGAGAAGAGACTTGAAAAGGTTTAGAGATGAACAATTAAAATGGCGACA
CACTGTAAAAAACAAGGGCTGCAGAGGCAGGGGCAGGTCAAAGGACCAGTCTGCTTGCTTAAAGCAGCGTGAGACGTAA
GCGTAGGGCAAGCTTTATCTTCAGAAAGAGTGTTACAACCCCATGAGCAAGGAACATGCCACTCCCCACCTCCTCCCCC
CTCCCCCACAAACCCACCCCAGGTAACCACCTTGAAACGTTTTCTTTTTTTGAAAGGAAAACATATTTTCTTTATTTAT
ATTTCAAATGTTATCCCCTTTCCTAGTTTCACCCCCTCCTGGAAACCCCTATCCCGTCCCCCCTCCCCTTGTTTCTGTG
AGGGTGTTCATAAGGGAAATTGGTCTGAAGTTCTCTTTCTTTGTTTGGTCTTCATGTGGTTTAGGTATAAGAGTAATTG
TGGCTTCACAGAATGAATTGGGTAGTGTTCTTTCTGTTTCTATTTTGTGGAATAGTTTGAAGAGTATTGGTCTTGGGTC
TTCTTTGACAGTCTGATAGCATTCTCCACTAAATCTATCTGGTCCTGGGCTATTTTGGTTGGGAGACTATTAATGACTG
CTATTTCTTTAGGGGTTATGGGACTGTTTAGATGGTTTATCTGATCCTGTTTTAACTTTGGCACCTAGAAAATTGTCCA
TTTCATCCAGATTTTCCAATTTTGTTGAATATAAGCTTTTGTAGTAGGATCTGATGATTTTTTTGATTTCCATGTCTCC
TTTTTCATTTCTGATTTTATTAATTTGGATACTGTCTCTGTGCCCTGTGGTTAGTCTGGTTAAGGGTTTATCTATCTTG
TTGATTTTTCTCAAAGAACCAGCTCCTGGTGTTGTTGATTCTTTGAATAGTTCTTTTTGTTTCCATTTGGTTGATTTCA
GCCCTGAGTTTGATTATTTCCTGCCGTCTACTCCTCTTGGGTGTATTTGCTTCTTTTTGTTCTAGAGCTTTCAGGTGTG
CTGTCCCCGCTGTCGTTGAAACTTTTTCTTGAGGACTTTGCCCAAAACATTTGCAAGATTTGGAGACTCAGAACCTTTT
AACCCCAGCATAGTCTCTTTCTCGTGGAGAGCAACATCTCCAAATACACCAACAGCATCAGGCAAAAACATAGTGTGAT
ATGACCTCCTCTGTAGCTGACAGATGACAGAGGAAAGCAGAACAAAAGAGGGGAACATTGCCTCGAGTGAGGGAGTGAG
AAACTCACCACCGTTCTGCAGGCTGACTGGGGACCCAGAGAAGCTGGGGACCCAGAGAAACCTGAAAGCTTGGGGTCCT
TGAGGTGGTTTTCAGTGGGAGTTTGGTCCCACCTCTGTCACCTGCCTGGCCTGAGTTCTAGATATGACACTCTTTGTTG
CTATGAGGAAGTTATCCGTAAACAAAAAGGCCCTTCCATTGTGATGTCCCATAAATGCCAACATCCACTGCTACTGTTT
ATCAATGCACTACTCAGCCATCAGAACTTCCCCAAAGAAGATCAGTAAGAACAGCCATCGAAATCCACTTCGATGAAGT
TCTGGCCCAGGATAGGAAGGCGAATGGACAGACCCAGATCAGTGAATCCATTTGTTTCAGGAGATGAACAGTCTATAAA
AACCTCAGTGGCCTCCAAAGGTCTCAGCCAGCACCTAGCTGGGCCATAAGAGAAGGCAAATGTTTCTAATGACTTCACT
GTAGCGTAGACCACTTCCTTCAACAGAGCTTCAGGCTGTCAGTCATTTTAACTCTGACAGTGGCATGGGTGTTTTCTGG
CTAGACCTTTACAACTGAGTAATGGGTGGGTTTGGGTTCCCAGGGTCAGGAGAATGGAGAGGGCCATGATGTCCAAACC
ATGGCAGACTCACAGCCGGCTCCCACCGGCTCCGTTCAGATTCTGGGACAGCACTGGCCTAAGACAAACGGGGGTATGC
AGGCACATCAGCCAGGCCAGGACCTCGTGCTGGGCTGGAGTGTGGTGCTGTTTCTTGGTCTTGCTTTGTTTGATTCTCT
TCTCTTCCTGTCTCGCCTGGCAGCACACCAAGTTCTATACATCCGCAGCCTCCCGCCTGCCGCAGGGACTGCCTGTGCC
CAGATTCCGTCTTCCACCCTGTCTGCGGAGACAATGGAGTCGAGTACCTCTCCCCCTGCCATGCTGGCTGCAGCAGCCT
CAACGTGAGCTCAGCAGCTTCTAAGCAACCAGTAAGGACAACGACGAAGGGGGTCACAGGGGTCGACAGAACCACATGG
AGCTTTGGGGCTCAGTGTGTCACCTCAAAAACCGTTCCTTGGCCGTCAGTATCAGCGTCTTAGCCCCCTCCACCATCTC
TCATCCTTCTCTGCCCCGGGGTCATGACCAGATGCCCACAGCCCAAAGTCAGTTAAAGATTGGCTGGAGACCAACGAGT
TCATTCTTGGGAACTTGGCAAAACAACAGAAACAAGTCCGAGCAGTCGCTCTCAGCGGAAACTTGTGACTGATACTAAT
GGCTCCCTGTGGCAGGGGGGAACTTCTGGGTTCTGGGTTCTGGGTTCAGGCCATATTACAGAGTCACATGGCCTGAGAG
CAGAGGGTCCCCTGTGACTTGGACCTTAGAGGCCAGTGCTGGGAGCAGCAGTCGTTTCAAGCCCTCCTCTCCCAGGAGT
GCCACAGAGTTGGCCTGAAGCCATGAAATTAAGTTCAAATCTAGGATTAGAAACAAAATCTCTCTTCACCTGGTCTTCA
AGGCCATGTGTCTCCTTCTTAGACCTAGACCTATGGGGACTTGTCTGACAATATGTGAGGACAGTGACCCCAGGCCTTC
TGGGATCAGGAAACAGCAAATGCCTCCTCCAGGTTTGTCTCTTAGTCAGAAATCCATGTGTGCAGCCACCATGTCAACC
AAAAGCCCTGGTCTCACAGTTTACATCCAGTCCGATAAATGCACACGATAAATGCTGAGGGACTTCCTGAGGTGACTTA
CCCTTGAGCAAAGACCAGAAGAATCCTATGGAAACCTGGAAGCCCATTGCACTCGAGGGAAGCACAGGTGCCAGTGCAC
CAGAGAAGCTGAGGAAAGGCCTGGTCGGCGCTAAGCGACAAGTGTCACAGCTATCGGGACAAGGGATCAACAGGTCCAG
AGCTGGGCAGGGCCAGGCAAGCCAGACTTAGGAGGGAGATTATTTCCCCTCTTAACTCCCTCTGATGAGAACAATGGAA
CGTCTATGTCATTACTATAGGATGCCCCCATAGGAAGAATGCACCTGTGTGTCGGCCATGTGTGTTGGCCATGGGCCAG
GCTAGCCCCCACTTACCACCTCTTGCGAGGTCACAGAAAAAAGCTTCCCCTGTCTGTCACTATGGGCAAAGGCCCTTGG
CTCTCAGGTAGAGAAGGGTGAGGAGCTAAGACAAGATGGCCCCTTAGTTTGCCTCTTAACTGGAAGAGTGGTGGCTTCT
GTGGTTTTCTTATCTTTATTTACTCTCCGCTGACTCATCTACTTCCAGAAATAATCTTGACAAGCTTCCAATAACAGCA
CAAGTACATCAAGACAAAATCCATTACCATGATAGCAAGGGAAAAAATAATAAATTATAAGGGGGATAAAGTCTTAGTA
TTTTAAGATCCAGATTGCGGGAGGTTACGGCAATATCGTGTGTTAGGAACTTCTGAGCTTGAGGGCCACAGTCCCACTG
ACTGACAGAGGGAGGCCTCCTCTAGCTCATTAGTTGGCTCAGCCCCATAACAGCTCTGACACCAACGTCATCATAGGCT
GGGCCATCAGCCACTGCAAGGAGGGAAGGCAAAGATGACCAGATGAACGGTGGCCGCCAAATGAGTCGAACAGCAGTGT
TGAGCCACATAGAATTATGATGAGGCAGGGTCCTCCTCTGGGGGCTCTTCTTAGGTCAAAGTACACCTCTTCAGTGGGG
AGTGCTACAGAGCATTCAAGAGACTCTGTTATTGTTTCCTCTACTCCCGGAACATACCTTCTCCCATAACAACTTGCGC
AACCACTAGAGCGGGCTGGACCGGGAGGAGAGCTCCCAGGTTCCAGGCGCTGAGCTGATGAGGGTGCTTTGCCTGGTGA
TGGGATCTTGGGCATTCAGATGTTGGAAGACTGCAGGAGTTCATCCCATGCTGGACTCTTGGTGAAGACCATTTCTTTC
TTTTTTTTATTTTAAAGATTTGTTTATTTATTTTATGCATGTGAGTGCACTGTCACTCTCTTCAGACACACCAGAAGAG
GGCATCAGGTCTCATTACAGATGGTTGTGAGCCACCATATGGTTGCTGGGAATTGAACTCAGAACCTCTGGAAGAGCAG
TCAGTGCTCTTAACCGCCGAGCCATCTCTCCAGACCGTGAAGACCATTTCTAATGTCCCCAAAGTGAAAGAGCCAGGAC
CCCATGGGGCACTCTAAAACCCTCATCTGACCTCTTCAGTCTTTGAATCCTAGCTTTTCCTCCATCCCTGGAGGGCTGA
AGGGAGGGCAGAGGGATTCGTTCATATTAGAGGGAGGCAGTGTGGATATGTGAAACATACAGAGGTTAAATCATGACGT
GGCCCCTCCTGTTCTTCAAGATCTATTTGAACTGTAGCTGTGTGACTGGAGGATCCGCATCGGCCAAGACAGGCTCCTG
CCCTACATCCTGTGCACAGCTGCTGCTCCCGTCCATCTTCCTCATCTCCTTTGTGGCACTCATTGCCTGCGTCTCCCAC
AACCCCCTCTACATGATGGTCCTACGGTGAGTGAAATCTCTGAGCCTCTGGCTGCTGCTTCCACACCCTCTCTTTGACA
GTACATCCTGTCTCCTACTCAGAGTCTCTCCTCCCTTAACTGGGGAGTCTTAACAGCAACCTACAAGTGTCTAATGGTG
AGAAGACAATAGGTACACTGATGGCAGGAGTTCCCTGTCCCAGGAGGAGCTCCTGGTAGGGAAGCCTGGCCCAGGCTCC
TAGTATCCATGAAACATAGGCCCTGTTGGCACCCCCATCTTTATTTGTGGGAAGTTAAATCATAGCCCGGGATGGTGGA
ACTGTCGAAAGGTTGAGCCGCAGGTTCAAGACAGGCTGTGGGTAAAGATTCACTTGTTCTTCAATATTAAAAGAGGAGA
AATCAGCAAGTCAGAAACTGAGGAAAGTTCCAGAAGCAAAGCGAACACTGTGAGAAAGGGCTGAAAACGTGAAATTTGC
AGGAGACTGGGGACTCAGTGCTCAGAGTGGAGGGACAAGGGACCAGGAACCAGGGTGGGGTCAAGAGAAGTGTCTGGTT
TTTAATAGAGAGCGTTCCCTCTTCTCATTCTACCAAGAAGGAAAATGTAAGAAAAGAAAAAGAAAAAAGAAAAGAAATC
TCACCCAAGTTCATCTAACCACCTCCTGCCTGCCTCTCTGACCTCCCATCCAGCCCCATCAGCTTACTTGGAAAGCAGA
GAAGGAGAAACACAAAGGTTGAAAACTCCCTATCAGATTAAAAGTCTCTACTTGAGTGTCTTGGGTGTTGCCTGACCTC
ATGACTCCCAGTTTCCTTGACTATGAAAGGAAGATAGTGATACCCACCTCCAAGTGTGAGTGTGAGCAATTACAAGGTC
AATCGCTTCGTAAACAGAAGCTCCTCTGCTGGGCGTGGTGGTGTCATCTATAAATCTAGTGCTTGGAAGTAGGAGAACC
AAGAGTTCAAGGTCTTCTTTGCCTAGATAGTAAGTTACAAGCCAGCCTAAGCAATATGAGACCCTACCTTACAAGGTTC
TCAATGTTTGAGTAACAATCCCTTTGGGGGTTGACTGACTCTTTCACAGGGGTTGCCTACTACGACCATCAGAAAACAC
AGAATTTACATTACAATTCATAACATTAGCAAAATTAAAATTAGGAAGTAACAACGAAATAATTTAGTGGTTGGGAGGC
TCACCACAGCATAAGGAACTGTATTAAAGGGTCGCAACCTTAGGAAGGTTGAAGACCACTGCCCTACCTCAGAAAAGAC
ATGGGGGCTGGTGTTCTCCATATTGATTAGGACCTATCTCTCCAGAAGGCTAAGCAATGAGGAAGGACCAGCTGCCATG
TCAATAATGTAGCTTAGTAACAGAGCACTTGGTTAGCACATGTATTTTACTCTATCCAAGTCTAGTCTCTTTCCATTCA
TCCCAGAACCTCAAACTACTAAATAGTACCATGAACATTCAGGATTACGTCCTCCCAACTTAGTCAAATCCCTCAGGAT
ACATCTTCATGGATACACCAAGAGATTTGTTTCCCAGGTCATTCCAGATCCATACGTTAGCAATGAAGATTGGCCATCG
AAGTCATCCATTATGGATCGGGCCATTGGAGACAAGCTGATACAGAGGGAAAGAACATCCATTCAACTGGAATTCCAGG
CACTGTCCACTGGGAGATCCCACATGGTCCCTCTTTCTCCTACTTGAACTCCAAAGAGCCTCCCTTCACTGGGTCATCC
CCCTGGGGATGTCTCTCAGATGCCGACTAACTTTGGCAATGGTTGTAAGATAATATGGAATGATCTGGAGGCCACTTTT
GAGTTCTTTTCCACTCTGTCCTCGGCTCCAAATTCTTTTCCAAATGATAATCACCCAGCCAGGAGTATGTGGTGTGAAA
ACGTGGAGAAGCCAGAGTCTGGTCACACAGCAGGCGCTTCTACCACTGCTCTGAGCTCCTCAACCCTGTGTGTCAGATG
CCAACCCCCTTTTCACTCTCCAGTTGAGGTGGCAGCTCTCTAGGGAGAGCTGAACCTCTTTACCTGGCTTTGCCATCCC
CCTCTTGAAGGAGAAAAGGTAGAAAGCAGGACCACGCACAGAATCCACAGGACCCAATGCAAATGAAACTGTAAAGACT
TTGGCTTGTGAATCATTTAAGAACTCCAAGACATACGTAAACCAAAGCTGGGCCTTTCTGACGGCAGCATCCTATGCAG
CTATTCAAGTCACACCTTACAAAGCTGGCCCAGGGCAAGGAAATGTTTCAGATCTGCACAGAAGCTGATAGCCCAGGGC
CCTCTTTTCCTGGTACAAGATATTCTGTGGGTGTTGTTCAGACCTGCTGTCTAAGCATCCAGCCAGAAATTCCTTTCTA
CTTGGTCTCTCTTGCCTGCTTTCTTTCCAAGAAGTCTCTGGACAGTCCTGAAAACAATACAGACTATTGGTTCTCAAAC
CCAAGTTTACATCCAACTCTCCTGGAATATCCACGTTTTTGATAGATGCCAGGCGGAGAATCTGTATTTTGAACGTAGC
CTGGGCGGTGCCTCTGATCCAGCAACAGTGCTTTGAGAACGATGGGTATAACAGGCATTCAGCAGATAATGCGCATCAT
CCATGTGTCTGGTCAGACTCCATGAGGACCAGTGCTGACCTGCTCTGAGTCTTTCTACCCCCTACACCTCATACCCTCC
AGGTGATGCTGCAGTCTAGAGTCCTACTGGTGCTAACTTCCTGTTTTCCTTTTCAGTGTGGTGAACCAGGATGAAAAGT
CGTTTGCCATTGGAGTACAGTTCTTGTTGATGCGTTTGCTGGGTAGGTGATTTAGGCCCTGGACCGTGGGCATCTAAAA
GCCATGTGGGGCGGGGCAGGGTTTGGAGAAGTCCCAGTTCATCTTGGCTGCATATTCCTAACAGCAAGAAGAACCAAAC
CCTGACAGATGACAGTTTGCCTGATTGCTTGAGATCGGGGAAGATTCTGTCTACAGTATTCAAGCCAAATTTAAATTAT
GTAAGAAAAAGAAAAGGTCATAGCTGAGAGTGCTGAATACAGAGTTAAAGGCAGGTATTTTTAAATCTGTCTCCAAGAA
TCCGCCCTAGGAAACAAGACCTTGCATTCCTGCCCTAGACAGAGCAGTTTCTGAGCTGAGCCCTGGCTTCCCTCCCATT
TTCCAAACCTCAGTCCTCTGCCACGGTTCAGCAAATGTCTCTTCTGTCAGGGGATCCTGCCTCCAAACCCTGTCTGTCC
TCAAGTAGCCTGGAGCCCCCAGCTCAACACTCAAAATACTCAAGCTGTGCATACACATAATCAGCAGAGACCTACAGGG
AGAGAGAACTCCCACGGGAGCAGGAGGCATCTGGCTTCCCTGAGTGCAGGGACTGCAGGGAGAGGAGACACACAGTAGG
TTCCTTCCCAGCTGGCCAACTCTCATCTGCCCGTTGACATCCATGGCACAGTCATGCATTAGACTGAACCCCAGGCACT
GATGTGGAGGTCTTGCTACAATGGATGAACAAAAGTATAAAGATTGAATTTAACAGAGGATCTTTGTACTAAGAATTTC
AGGGGTGGGGATTTTGAGGTTCCGGGCAGAAGGACTTGAGTTACTATCAATGTGAGCCTTCCTACTTTAACCACTGACA
CCCTCTGCATTATTATACAGGGGTGCAGGTCACATCTTGCCCTGCCATGGCTTCCATTAGCCTTCCAGCGTGCGTCATC
ATCCCAGCTGAAGTCAGATGGGCAGGTGCCTATGCAGGGTCTGAGCCAGCATTTTCAATGGAGTTTCTTAGCCGCAAGC
CATTTATCCTATCATGAAGCGTCTCAGTTCCAAAGGACCCTTAACCCTCTAAATGACAACCGTCACATTCCTGGAACCA
CACAGGGGACACAGGGTATCTTGTCCAGGTTGAGGCTGGTTCATTAGCACAGCTGCTTGTATTTATTTATTTATGACTT
ACCATCTATAGCTGGTTTATCACTTACTCCAATGCCCTGGCGGGGAAAGAGCCCTGGCATTCCTCCCAGAGGACCTTCT
GCCCTGCTTCTATTGGTGACTTAGAGAATGGCCCCTCTGCCCCGCTGCTCCAAGCCCTTGCTGCTTCCTCTTTCCCACC
TGAGCTCAGCTAACTCTGTGGACCACAGTTGGAAAACACAGGCCTAGAGGAAATCCACTGGTCACTTAAGAACACAGCT
TCACTCTGGGTTAGATGAAAAGACTGCCTATTTATAAACCCCAGCAGGCTCACAGTCACCCACTGAGATGTGGTTGAAA
ACTCTCCTTGACTTCTTTTCTATTTAGGGATGGGTCACTAGGGCCAGTTTCCAGCCACTGTTCACCTGGAAACTGGAGT
GTGAGCAGGGGCTGAGGGGCTGCTCTTGGGAATTTCTCTATGTAGGTAGGGAACAGGACTGGGTTGCCTCTTTCTTCCA
GAAACAGGAACTCACAGGGTGCTAGAGTGTCTGGTGTTTTCAGCACCGCGTTTCTGAGAATGGGATGGCCCATCTGCAA
CGTAAATCCTGGACAAGTCCACCCTGATCCACTGACTGCTGGACAAGTCCACTCTGACCCACTGGCGGCTAGACAAGTC
CACCCTGACCCGTTGGCTGCTCACAACGTGGCTAGCACCACTGAGTTTGGTGGGGTTGGGTAGTGGGCATCTAAAATAT
TCATCACCCAGTAAGGTGTTGTGCCTGCTGGACTTCATCTTAACATTCAGATGCTAGGATGATTTGGCAACCTGTGTTT
CTGAGCAAGACGGGGAACTAAAGGTCTCCAAAGGGTGTTGTCCGATCTAGTGGGCTAGTTTGGAGTGGGAGAAGAACGT
GGGATTTGAACTTCAGTGAACCCTTGGGTCCTCTTGTTCGCTTGCACTTTATTCCAGTGACTCCACCCACCCACAGAGG
CTGGGGGAGTCCTTGGAGGCGTTAGCACTCTCGAGATGGAACTTCTACCTGGGGCCAGCCTTGAGCTTGAGAACAGTGC
TGTGTGTCTGAGCTGACTCCAGGTTGAGCCCGTGATTCATTCTGCCCTGAAAGAATGGATTCCCTTTTATCTCCTAAGC
CTTCTGGTGAAAGGGGAACTCTTGCTGAGCTTTGCTGCAGCTTTCTGCTTTCCCGAGTCACTGTGATTCCGAGGGCAAT
GTGTGAACCGTGCCATTGGTGTGGGGGGCTCGGACATCTCTGACTGCAGATCCAAGCCACCCAGACACCCTGAATTCTC
TTTCCCTTTGTTGCCAGCCTGGCTGCCATCTCCATCCCTTTATGGCCTGCTCATCGACTCCTCCTGTATCCGGTGGAAC
TACCTATGCTCAGGGAGACGAGGGGCCTGTGCGTATTATGACAACGATGCTCTTCGAAACAGGTGAGAGCTCCCACATA
CACAGGTGTCTAGGAGATTAGGTCTGTTGTCCCAGCACGGGGGGGGGGGGGAAAGCCCTTTTTTTAAACCTCATGGATG
TGGTGGAGGCTGTCAGCTTTCTAAGGGGCAAAATAGGAGGGAATGTCTCTATAGATTCTGCCATGGACAGAGAGCAGAG
GCTACCATAGCAGGCATTTGGGGACCTATAAGGAGCCATTCAGGTGAGGAGACACAGGGTCTGCTTTGAGGTGTCCCCC
AAAGTACTGTCCCTCACAAGCCTACTCATAGCAGCAGACAATGGTCACCAAAGTTAAAGGTGTACCTGGAGCCTGAGAG
AGATTTCGAATATGGAAAAGATACCAGTCACTCTCCACTGCTCAGAGAATGGGGAACACAGCACGGCGCCCAGGAGAGG
TCCAAAGAAGCAGGAGTCCAACCTATGCCTGTGAGCCAAATGCAGCCATCTCTTCTGTTGCTGCCGTTCCATCATCTAG
AAATGGTGGAGAAGGTGGGCCTGCAGGGGCTAGAAATACTGAGGACGGATAGAAAAGGCCAGGTTAGAGAAGGCCAGCA
GAGAGGCAGCTGAAAGATACAGACCCTCTGGAGACAGCTGGGACAGTAGTGTCACCTCTGGCCATCAGTGATGTCACAG
GAGAGCAGAGGATCACTAGCGTCATGTGTCTCTCCACAGTGACCACCTGAGAAGTGACAGCTCGTCACCTGAGTAACGA
TCTTCACAGCTTCTAGTGAACTGTGAAAACAAAGATCATCTTCCCGCTGCCCCTGGGCCTCACCATACCTGTCTCTTCC
TGCCTCATAGGTACCTGGGCCTACAGGTAATCTACAAGGTCTTGGGCACACTGCTGCTCTTCTTCATCAGCTGGAGGGT
GAAGAAGAACAGGGAATACAGTCTGCAGGAGAATGCCTCGGGCCTCATCTGACCTTCAGCTGGGACTACTGCCCTGCCC
CAGAGACTGGATATCCTACCCCTCCACACCTACCTATATTAACTAATGTTAGCATGCCTTCCTCCTCCTTCCCTCTCTC
TCTCCCTCTCTCTTGCGCACACACACACACACACACATACACACATACACACACATGCGCCAGTGCGCGCACAAAAGAG
AGAGAGAGAGAGAGTTAGTTCACTCACCCCTTGTCTCACCGGCCTCTTCCTCCTAAAGTCTCCCGCATGGTTCTTAGGG
CCTGAAGGGTCCTGAGCTGAATGAGCGCTGGACTGAAGGGTCTAGTTTCTCAGAGTCCTTGGAAGGGATCCTCTCCTCA
GCCCTGGGTCTTCCACTGTCCGTACTAAGGCAGCCTCTGTTCTCACCAGTCTCTCAGGAACACTTCAGTGGTGGGAGAA
ATGGGAGCCTTGACTTCCGTGCCAGGCAGCCCAGCTCCTCTGTCTCTGCCCACACCCAACAGAGAGGCAGGGGAGCAGC
AGGTGGTTGTTTCTTAGCAGCTACCACTGGGCTAAGGGAGCTTGGCTGTGGCCGCTTTGCATCTGGCTTCAGTCTAGCA
TGGAACCCAGGAGGGACCTATCTCCCTTTCCTTTCCCACTTTCTGCCCTCAAGGCTGATTTGGAAACCACCACTTTGGG
AAGTATAACCCTCTCTGTATACTACTCCCTGGTGTCAGAGCAGGAACGAAGGAACCAGCAAGGCTAGGTCATCCAAGCA
CTGTTCCTAGGAACCTGAGGCCTACCCTCAGGTCAGCATGTGCTTCCTTCTGCCTTTCAAACCAACAAAGCCCATGTAA
GGAGCCCATGTAAGGAGGACCCAGTCTCCCACCCTCACAGTAGAGACAAGGTCTTTCCCCCTTCAGAGACGCAAATAGC
CATCTTTACCATGCCCCGGAAACAAAAGAGTGACCTCACATGGGCTTTGCTGTCTGGACTTCAGCTCATGTGCAGAGGA
GTAAAGGCGGTGAATGGGGGGTGTGATCCTGAGATGCAGTCAGACCAAGGAATGGAGCAGAGTGGATGCCACCTGTCAC
TGTGCCAGATGGCAAGTTGCAGCCCACAAAGAGAGTAGCCGGGTTGACTCGCTTGCAAACACATTGTCTTCGTTGGCAT
TTTCTCAGCTTTTCCTCACTACCTCTCTGGATGTGGAGGTGGGCTCCTTCTGTGGGCCTGGGCTGGGGGCCACCACTGC
CATGCAGTGGAGAGGCAAAGGCAGAGGCCCAGGAATGAGGCCTTTTCTCAACGATTCTGTTTATCAGAGAACTGGCTTG
GTGTTTGTCAGGGATGCTGTATGGTTCTCTGAGAGCTCCCCTGGAAAACCTTACCTCCATTCGCATTGGGATTGCTCCC
AGAGAGCGCTCGGTGGGCAGCAAGAACATGCTCACACACTTGCCAAGCATATCCAGAACCAAAAAGGCTGGCGGAAAAG
ATCCTGGTCTGACTGCCCATGCACACCCTGAGGCTCCAGCAGGTTGACTGTGTGCCTGAGCTGGAGATCTGGCTCACCG
TGTCCCCGAAGCAGCTAGGCAATTGACATTGCCCTATGTGACATTTAAGTGCCTTTCAGAAATGCCTTATATGGTGAGA
AGCCAAATGGTTTATGTCGGCGTGGCAAAGGTCCTGTGAGACTGAAGCCTTCAGGGAGCCTTTGTTACTGGCAGTGTTC
TTTCTGAAGCCACCGGGCTCATTTCCACAGATCTTATTAAGGAGGAGGCTCCACACAACCTCACGGGCCAGTTTCAGCA
TGTGGAACGGTTCTGCTTGGAACCTGATTCTCTGCAACTAACCCTGGGCTTACTCACTGCAGTTTAACCAGATTCTGAA
GGCATAGCCTTCCACCAATCTCGCAAACAATGAACGTCAACCTTCCGTTCCCGGAAGATCAGCTGTCCCCACAAGTCTT
TATTCTATTATATTGCTATTAATATATTATAATTCTGTTCTGGGTGTAAGTTGGTTGTAAATTTGTGCTGTCCTGGGTC
TATCAGAGAACAGACAGAATTTGTGCCTTCTAGACAAAGAAGTAGCCATGCTTTAAACTTTAGAGGCCTGTAGCCAAGG
GTGTTAGAGACCATGTTACTCTCAAGCCACCTGCCTGGTGCTGAGCAGGTCCCAGGCATTGCTGGTTGCTGTGTGGGGC
CTTGTGCTTTCTGTGCAGACCAATGTCAAAGACTCATGCTCTTGGGAGGCCCAGTCACCGTCCTTGAGTTCATATCCAG
AAGTGGGCTCAGTCACAAACACTATAGCCATGAATATCACAGTCATAATTGATCATTTACTACTATTCAGCAAATCTTG
CCTCTCAAATGCCAGAGGGGCTTTAAAGATCTCACTTTTTTTTTTCCAATTAAAGAGCAAATTGTCACAGACTTCCATG
AGTGTATCATGCTCAAGATTGCTGTTAAAATACAGGAACACCTGGGCTGGAGAGATGGCTCAGTGGTTAAGAGCACTGG
CTGCTCTTCCAGAAGTCTTGAGTTCAATTCCCAGCAACCACATGGTGGCTCACAGCCATCTGTAATGGGATCTGATGCC
CTCTTCTGGTGTGCAGGTGTACATGCAGATAGAGTACTCATACATCTTTTTAAAATGCAGGAACACAGCCGAGCAGTGG
TGGTGCACATCTTTAATCCCAGCCCTTGGGAGGCAGAGGCAGGTGGATTTCTGAGTTCGAGGCCAGCCTGGTCTACAGA
GTGAGTTCCAGGACAGCCAGGGCTACACAGAGAGCCAGGCTACACAGAGAGCCCTGTCTTGAAAAACAAAAGAAACAAA
AAATGCAGGAACACCCTTCCCAAAGCCTGAGGCCCCTTTCTGTGACTGTAAAAGGAGTCCTGAAATTCCACCTTCCAGG
AAGATGAAGGTAACCCCTGAGCCCTGTGTGATGTGGGTCACCCATGCATAGAGCCAAATCCAGGCAAATTGACCGTAAG
GATTAACCACCAAAGGTGCCTTTATATGTAAGAAGGTCATAGTAGGTCTTTGATCTCTTCTCCTTGCTTCTCTGTGGAG
CTTAGGAACCCTGTAGAGTCTGGCTCGGGGACAGAGTTCCATTCCTGGGCACAGCCAGAGCTATCATTCTCTGCTGCCC
CACCCAACCTAGGAAATGCACCATCCAGCCTCACAAAGAAGAAACCAAAGAAACAGGAAATAAAGGGTTTGCCATCCTT
TGGAAGGGAGCGATATCTACCAAGTGTCCTTGAGTCCAAAGCCTCTGGCAGGACCCTCGTCCCAGCCTATGATGGGAGC
CACACTAGTATGTCTGAAAATGTAGAATCCTAGGCATGTGGAGCATGCCTTTAATCCCCGTACTTGGGAGGCAGAGGCA
ACTGGGTGTGTGAGAGTTCGAGACCAGCCTGATCTACATGATGAGTTCCAGGACAGCCAGGGCTACATATTGAGACCTT
GTCTTAAAAAAAGAGAAAGAAAGAAAGAGAGAGAGAGAGAGAGAGAGAGAGAAAGAAGGGAGGGAGGGAGGGAAGGAGG
AAGGAAAGAAAGAAAGAAAGAGAGAGAAAGAGAGAGAGAGAAAGAAAGAAAAAAAAGGAAGGAAGAAAATGAAAATGAA
AAAAGAAAAAGAAAAAAAGAAAGAGAAAAAGAAAGGAGAACTCCAAAATCAAGGAAGCAGCCAGCAGAGAGCTTTCACA
GAAAGCCCAGGTCTTTCTGGCTGCCCCAAGCCCCTCTCTTCTTATAAGTCATTCTGAAGTTAGGGCAAGCCTTGGGTAA
TGAGAAGCCCGCCTCTTTTCCCATTGAAGTTCATTGGCTGGTGAGAAGAAAGAGGAGGGATCTCTTTAGGGTTGTGTAG
GTTGGGTGATAGAGGTTGGGAGACCAGGTGATGTCTCAATTATTTACATTCTAGTCAAGCATCCCAAGAACATTGTAAC
TATTCATCAAACAAAATCCAGTGAGCCCTCTGGGTACCACAAGGTAAAGACCAGACGTGGAGAAAAGGGACAGAGAGGA
GAAAACTTGGAGAAGCAGAGCCATGAGATTGAAGCACAGACCTTTGGGGAAATGTCCCATCTGGTAACTGCTTATCATG
TTCTCGCTTAAGAAACAAAGCCATGTTGTATTTGCCTTCAACACTTTCAAAGATGGAGAGATGCAGCTAAGCTGGGTCC
TGAATTACTGTCAACTTGATAGTAAAATATTGTATTACAGCTTCCCAGAGAGATTGGCAGTGGTATGTGTATGTGTGTG
TGTGTGTGTGGGGGGGTAATCCTTGCAAATCCAGAAGAAAAAAAATTCCTATCTTTCCCAGCCACAGGGACTAAACATT
GGAGGTAAAAATGTCCATTCCCTGCCAAGAAGGCCAGATTTGCTAACCTTTCATCGGTGATTAAATATGTAAACAGGTC
CTGGGCAAGCCCTTGGCAAATCATTAATGAGAGAAAGCCTTCCTCTCGTGACAGGCCCGGCCCATTTATCAACCTCCAC
ACCCATTCCCAGCCCTGTTTGGTGGCCACAGGACACAGGGCTCTCTACCTTGACAAAGTCTCTGCTCCTTAGGCACCTG
CTACACTTTGATAACAATAATAAAATGTACATAGTTCGCTACAGTTTGCCAAGCCATGCATTTATTTACTGAACTCCTC
AAACCACCCAGAAATAGGCTTGGCTAGGCTTCAGTCACATGACACAACACACCTTTATCTCTTGTTTGCACAAAGCCCG
TGGCTTGTTTGGGTGACTCTCCAGGGCAGTGGCTTCCAAGGGTGGCTCAGAATGCCAGCTATTTGGACTTGAGGTACCT
CTGTTTCATCAATCCACAATTAATGAGGCAGGAAAGGAAGCTAAGAAGGTCTCGCCATGGGAAGCAAGTGTTTTGGCCA
GAAGGCGCATAGTCTTTCTGCTCCCAGGCGCTTGGCCAGAACTAGTCTCACGGCATTATCTAACTGCTGTACAGATGGA
GAAATATAATCCTCTGTGCTCAGAGAGGAGAGGAGCAGACTTAGGCAAGTGCTAGAATCTTCCACCATGGGGAGTGATT
CTAGGTGAACCAATATGCTCTCTGAGACCCTTCAGTCTTGTCAGGCTGTTGGAAGAGGGATGAATACAGTCAGAGGGAG
GAAGCATCCACAGCGGATCCCTAGAGGAGAAAAGATGATATCTCAGCAAAGCCAAGGCTGCCTTAGAGATGGAAAAGTG
GAGAGGACAGGCCCAGAAGAGGCAGAGCTGATGGATCAGAGGTTTAGAGATGCAGGGTCAGGAAAAAGGTTCTCTTTAG
TTGACAAAGACTTAGGCAAAGGATGAGAAGGCGGAGGAGCTAGGGGGGATGACAGTTGATTGAGCATGCCCCCCAAGGG
GCGGGGCTTATCTCGAGGGGAGGAGTCTCACCCAGAGCTTCTTCCTGAATGGTGAGGCCATCAGGACCATCACATCGAT
AAATGAGGTCCCCTCCGGTTGTCAATCACATTTTATTTGTTTGGTAGCTGGTGTGTGCAAGAACCCGCCATGACACATT
TCCTGACCTTGCAGCGTGTATACTTTGACCTAGGGTAGGAACTTGACCAAGTGCTGTGGATTCCATTGGGAAATCAGAG
AAGGGACCAGGGCTGGTTTCGAGGGTCAAGAGTGTAGGGAGAGAGGACAGCTAAGTTGTGAACAAAGGTAGAAAGGCGA
GCAAACCTGTGCACATTCTGAGAATCCCCTGATCAGGGAACCTGAAGGAGAACAGGAGTAAGGCTAGAAGGGTAGGGTC
TAGGCTTAGCGGGGGCCCCTTAGGCGTCCTTCGGGACTGGCTTAGACCAGCCCATTCAAAGCCTGGAGAATGGCCTTAG
AGACCAGTCAACCAGTCCCAGAAGGGCGTTCCTACCTCAAAATACAGCCCCGGGTCCCTGAGAGGGAACTGCTACACCC
CAGAGACCAGAAGAGCCGTCCTCCTGAGCAGCCCCAGCCAAAGTTGGCTCCACTATATGTCCTCTCTCTCTCTCTCTCT
CTCTCTCTCTCTCTCTCTCTCTCTCTCTCCTCCCCGCAGTGTGGGAACTGGAGTCCCAGGGGTGTAATTGAAAGCCAAA
TGGGGCAGCTTATTTGAACAGGTTTTCTGTTCAGGCTCCTAAACTTCTCTTTGCACTGTGGGTTTCTCAGTACAGCATT
TCAAATGTATACAGCCCCTAAACCTTAGAAGTCTCAGGTGGGGCAAGCATTTGACTGAATGTTCTTAACTTTCACAAAC
ACCAGCTTACAGAAGAAAAATGGAGGTGGGAGTGGGGTGGGGTGGGGTGGGGTGGGCCCATACTGGGAAAAGGCATTTG
AAACTCCAGAACAGCTCCAGAAGTCGGGAGTGAAGATCAGAGAACGCCAGGCCCTTTGTGCTTTGCGCTTCTAGTGAGA
TGCAATTATCAGACGGGGAGTTTGGAGTGCAACTGGTCTCGCTTCCAGCAGGGTCCAGACCTTATTCTTGCAGTGGCCG
GCGCTCCGGGGCACTGCTGTCGTAGTCTGCCACTAGGCGGCGCCGCTCACCAGCGCCGCAGTGAGGCAGCGCCACAGCG
GGATCACCGCATGCAGCTTGGCAGAGAGGGCGTTGCTGAGGCCAGGTTGCTCGCTGCTCAGGGCACCCCTCAAATACAC
CGCCACCCTCTGACACTTCAGGCTCCCCACCCAAGCATTCTTACAGACCCTGTGGGCACTCCCAGGCCCTGCGAACTGG
CATGGGTGGTCACTCTGCTCCAACGTGGGGCCCCTCCCATCCTGGGGCTGTCAGCCTCGCCATGATCTTACAGGCTTAA
GTATTCAGTGGCTTCAGGGAACTCAGTCACCTTCCACCTCCGCCTTCTAGGTCTCCGGTATTTACCAGTGTACCCCTTG
GGCCTCAGGAACGTGCCTCTGAAAGAAATCTCTCATAATCGCAGGCACTGCCCGTCTTCAGAAAAGGTCTCTGTGCCCC
AGTCACCTTCTAGGGCATTCGGAAAAGGACTGAGCTCCCTAGGGCCAGGATAAACAAGACTAACAACACTCTTGCTCCT
TGGAAGCAAAGGCAGAAGTAGCCAGGAAAGCTTTAGAAACTGACGCACCCTCCTGACCCCCCAGGGTTGGCTGATCAAT
TCCAGGTTCAGCAGAGCTTGAAGGAGACCTTGACCGACACCAGCATCCCGATGCAGGGGAAACTGGATTCTCCACCTCT
TGCCTTAAGCCTCCCTTCCTTGACTTCTCTATGTCACCCATTAGCATTCCAGCCTTAAGTGATTAGGCCAAAATTTTAT
AAACAAGATGAAGACATATTTTTAAAGCCCGTTCCTGGCAATGTACGACTAACATCATTTCACCGCTTATCGTTCCGTA
CATTTTATAAATGTAGATGAGAACACAACCACCGTGGCCATTAAAGTGTAAAACAGTGTAACTCCCAGCAACTAGGTGG
ATATTGGGTTGAGTACAGTCTCTGCCCATTTTACCACCCTGCCCCCTTGGTATAAGTTAACCTAACTTATTCGAAAGCT
CAATGTAAATGGTATTTTGCAATCCATCCTAATGTTTTTGAGATCCATTGCTTGTGCCTGTGGTTATTCTTTTTTTTTT
TTTTTTAATTGTTCCGTGACACTCTCTTGGTTGAATATGACACAAATTCTGTATCTATCCATCAGGTTATTTAGTTAGA
TGTCAGGCCATTTTTTTTTTATTATGATGAATAAAATTACTATGGAGATTCGTGAGTGAGTTTTTTGTGAGTTTCCATT
TCTGTTGGAGACATGTCCAGAAGTGAAAAAGCAAAGGGATAGCTCATAGGCTATCAGAGATTAACTCTTTCCAAAGTTG
AATGGCTTAGCATCTCACTAGCGATGCAGAAGTCTCTTAGTGTTTCCAAAAGGAGCTCTGTTGAGGTTTACGTGGGGTG
CAGTCATATCCACACATTCACAGTCTTCTGTCAACTTGTTTGGATAAATGTATGTGTTTATCCTAGACTGCTGTAGCAC
CACACATGGGCTCATGTACTTGAATATGTACTTGAACCCCCAGTTTGTGGCACTGTCTGGGGAGAGAGCTCAGCCTTGC
TGGAAGAAGTGCCCTGGGGAAGCCAATTTTTTCAGCCTCACCCTACTTCCACATCCCTTTCTTTCTACTTCCGGTTGCC
ACTGAAATATGATTTCTAAGATTTCTCCTCCAGCTCCCAGTCCTGCCCACCTCCCCCCCAAACAGTAATGGACTCTCCC
TAGGAAGAGTGAATCAGGCTTCGTATTCACTGAGCTATTACATCATAAGTGGTTTTGTAATTCATATTTTATACTCTTT
CACTGTGGATGACCTGAGACACCTGGAAGATCCCTGGTCGTCATCCTCCTTCGCCTTTTTTTTCTTGTCGGTCCTCACA
TTACCCTTTCAGGGAGTTGTCTTCAAACTCATCAAGTTTTCTTCTGCACCCTGTAAATATATTCTTGCAACCACCCACA
CATATGTTCATTTTTGTTTTATGTTTCTCAGGGCTAAAAATACTGTTTCTTTTTTTATCACTTCCATAATCTCTCTGCT
GAGTCCCGGTTTATTGATTCAGATTGTAGTCAAAGACTGGAAGGTCTTTGAACTATTTATAGGAGCTACCTTCAACTCC
CTGTCTGCTCAGGTAGACATCTGGGTCATCTCATACCTTGGCTGCTTTGATTTCTGGACTCTGGCCACATTTCCCTTTT
TATTTGCTAGTCTAGTTCTTTTTAATATACCCTGGACATTATGAGTGATGTTCTGTAGAGGCTCCAGATCCCATTATTG
TTCCATTTCCCTGATGTTTGGGATGGAACATTAAGGCCTTGCAGATGTGAGTCAGACAATCAGCCATTGACCTTCATAC
CCAATCCTCTGTTATTATCTCCTAAGGAACCATGGTTCATATATATATATACACACACACACACACACACACACACACA
CACACACACATATATATATATGACTGATCACTTAGGACAGTGGTTCTCAACTTGAGGATCACGGCCCCTTTGGCAAACC
TCGATTTCCAAAAAACATTTACATTATGATTCATAACAACAGCAAAATTGCAGTTATGAAGTTGAAGTAGCAACGAAAA
CAGTTTTATGGTCAGAGGTCACCACAACATGAGGAACTGTGTGAAAGGGTCACAGCATCAGGAAGGTTGAGGACCGCCG
ACCTAGGGGATGCTTATTTGATTCACTTTGTCACCAAGAGTCAGCAAACCCTAAGATAGTTATCAGATTACCCAGCCTT
CAGATAGTTAACAGATTACCCAGCCTTCAGATAGTTATCAGATTACCCAGCCTTCAGATAGTTAACAGATTACCCAGCC
TTCAGATAGTTAACAGATTACCCAGCCTTCAGATAGTTATCAGATTACCCAGCCTTCAGATAGTTAACAGATTACCCAG
CCTTCAGATAGTTAACAGATTACCCAGCCTTCAGATAGTTAACAGATTACCCAGCCTTCAGATAGTTAACAGATTACCC
AGCCTTCAGATCTGTCTCCCTTCAGGGTCTCCTGCAGGCTTGAATGTAGGGATTTTGAGGCGGGTCTAGAGTTGACACC
CAGATGCATCTGTCCGTGCTAGCATCTTAAGGCTTTGAGGTTGTAATGAGACAGGGACTGGAGAAATAGCTGGCATTGG
GGTCACAACTGACCACCCTTCTACCTATGGAAGGGAGCTTTCTTTGGGCTCCTACCTGCTTTGTCTTCCTAAAGAGCAG
CCAAGAGACACCCAAGAAGACTTTCAGGATCACATACATGATCCACGTGTGTCTGTCTGTTGGACCTGATTCTGTTACA
TGGATATGCAGATGGACTAGACTAGCCCAAATGCCCACATTCTTCTGTTGCAGGAAATACTAAGCTTATTGAAATAGGA
GCTGAACATTTTCCTTTTGTTTTGTTTCTTTGTTTGCCCAGGTACATGAGGATTCACCAAAGGTGAAAGAGCCACACAC
CGAGTCTTATGTCTCTTCAATGGCTTTGTAGTCACAGTGTCTGAAAATGAGTCACACAGTCGGACCCCGATTGCTTGTC
TGCGTTTACAGTTGTCTGAAAGATGGGAAGAAGGTTCTCGAATTCTGCTTTTAATTTGTTGGACTGAAAACCCAAGACC
TCAGAAATAGTTTCCTACACAGAGCAAAGTAGCAATAACCATAAAAATGATTGGTCAGTTAAACCTTATTGTTTGCGTT
TCCATTGCTTTGATAAAACCCTGGGACAAAGAGCAACTCAGGGAGGAAAGGGTTGATTTTCATCTTACTATCCTCATCG
TGATTTATCGCCGGAGGGAAGTCAGAGTAGGAACCTGGAGGTAGGAGCTGATGCAGAGGCCATGGAGAAGTGCTGCTTC
CTGGCTTGCTCCCATGCCTTGCTCAACCTGCTCTTTTATAGAAGAACTCAGGACCACCATTCCAGGCCACAATGGGCTG
GGCCTTCCTAATCACACTAGTTATAATGCTGCCAATGCATAAACACACGGCATCACAGGTAAATCTTGCAAGTGTTTTG
TAGAGTGAAGGCTGAAACACAGAACACAGAAACAACGATTCCAAAAACAGACAAACAGCAAGATGGAAGAGGTTAAATT
CAGAGGAAGAAATAATAGTGGCCGGGAGGAGGAAGGAGTGTTGATTGTAGCAATGTTCAATTTCTTGACCTCATGGTGG
GTACATTGTGCTGTAATTCATTCCAACGTGCATTCTTACCCCTTGTTCTTTTGTGTATTGAACTTCCCAGTATAAAACA
TTACAATGCCATGCATATGCCTCAGTTGGACACGATAATAGTTGCCCCATAAGCCCAGGAATTAAACAAAGAATCGTCT
TTGAAGTCAAATTCATACCTAATATTTTCTACAGGTTTATTTAATTAGGCGTTTTGATGGATCTCAGATTATAGTCAGC
ACTCGGGGTTCTATTATATTAATAACATGCTTCAGATTTTGGAAAGAAATGTTTTCATGTTTAAAATGTCCTGAGTAGT
GAGCACTCTCCATTGTGGAATTCATTCCCCTTTGTCTCCGTTTATGAAGTTGTTTGAATGTGCACTGCTAAGAGTGCGC
TCCAGGGGAGTGCCTGCTCTGAACTTCATTTTCCCTAGACATGGCCTCGAGCCAAAAGTCTGATTTTTTTTTCATACTG
AAATCTAAGAGAAAAGTGACATTTCTTCCTTGCGTTCTCAGTCTGCTGTCATGAGGAGGAAACAATTAAGTTGTAACAC
TAGGCATAATTTATAATTACACACTAGCCTATATTGCTTGAAGCTCATTTGGAACAGAAAATATTACAGTTAGTAGATT
TTTAGCCCCAAACATCTTTATCTATTTGCCTCACCTCAACAGAGTGCAGTATTTACTTATTAGATGAAAGCTTAATTAT
TATTATTACTATATGGCTTAATTTTACACTTGCAAAGTACTTATTCCAAAAACGGTTTGGTTTGTTTCTAAAAAAACAA
AAAACAAAAAACAAAAAAGGCTCTATCCATGAGTTTAAAAAAAAAATAACTCATTTAAATATGCTTACCCACATCTCCA
AGCTTAAAACCCCTGTTTTCACTCAGCTTTCTATGGGGTTTCCAAGATGGCCGCTCACCCGCCCCACCCCTGGCTGAGG
AGACACAAGGAAGGCAGAGGAAGGAGTGTGCCCATCGCCTCTGCTGTGAGGACTGCGAGTCAGGAAGCTCCAAACCACT
CTGGTGTTTATCTGTGTTGCCACAGCTTTGGGCTGATAATCTGCCAGGGCTTAGTGGGTCAGAGGTGGGGTGTCAGGTA
GAAAGGTGTGATGGTTTTCATCTTGACAGGATCTGGGATCACCTAGGGGACAAGCCTCTGGGCGCAGCTCTGCAGGATC
ATGTAACCTCTGTGACAGCAAGCAGTTGTGTTGGATTAACTGGTGTGGGAAGACATGGGCATTGAGTGAGCAGCCTCGT
TCCCTGGGGTCGGGTCCTGGGCATTGTGAAAAGGAGCAGCTGAGTTGAGTACAAGCTCTCTGCCTCCAGCTGCTGGGTC
ATCACAGTTCCTTAGTTATCATAAAAGAGATGCCATTTTTATTTTAAAGTGAGTCTGCTATAAATTCTTACCTAATAAT
AATAATGCTCTTAAGGAGGGAAGTTTACTATTGCTTTGTCTTTCAGTATTGAGGTCAAACCTTGTGTCTTTACCACCGA
GCCACACTCATAGCCCCCTCCCCCCCTCCATGCTATTACACTTTTTTACTCCTCAGATACTTAGTTTATAAACTCTGTG
ATCGGAGCTGAGACGAAAGGATGGACCATCTAGAGACTGCCATATCCAGGGATCCACCCCATAATCAGCTTCCAAATGC
AGACACCATTGCATACACTAGCAAGATTTTATCGAAAGGACCCAGATGTAGCTGTCTCTTGTGAGACTATGCCGGGGCC
TAGCAAACACAGAAGTGGATGCTCACAGTCAGCTATTGGATGGATCACAGGGCTCCCAATGGAGGAGCTAGAGAAAATA
CCCAAGGAGCTAAAGGGATCTGCAACCCTATAGGTGGAACAACATTATGAACTAACCAGTACCCCGGAGCTCTTGACTC
TAGCTGCATATGTATCAAAAGATGGCCTAGTCGGCCATCACTGGAAAGAGAGGCCCATTGGACACGCAAACTGTATATG
CCCCAGTACAGGGAAACGCCAGGGCCAAAAAAATGGGAATGGGTGGGTAGGGAAGTGGAGGGGAGGGTATGGGGGACTT
TTGGGATAGCATTGGAAATGTAATTGAGGAAAATATGTAATAAAAATATTAAAAAAAAGAAAAAAATAAACTCTGTGAT
CAATCACATCAAGACGAGAGGGACCACAACCAAGGATTCCTTGTCGAGTGATTAGAGAACAAATTCAAGACCCTGGCAT
AAATATGGTGAGGGGCCAGAGAGTTTATAAAGTGTTCCCTTGTACATAATCTTTTTTCCGTAGCAAATATACAAAATAA
AGCTAGCACTATAATTGTGGTTTCTACAGCCTCAAAGTGTGTGTGCGCGTGCACGCACTTGTGTGTGTGTTTGCATGGT
GATGGTGGTGTGGTGGTGGCAGTATTGGTGGTGGTGGTGTTAGAATGTATGAAACTGAACCCAGGGTCACACTATACAC
TAGGTAAGCACTACCACTGAGCTGTACCCCCAGCCCAGCCCCCAAAGCCGCAAAATTAATGACTAGATTCCTTCTAGAA
TTAACAAAGAAGCCTAGAGTTAAAGTGCAGAAAGTTAGAAATCTTAAGAAAACAAACTACTTCCAGGTTATGACATAAT
TAAAATAAACAAAGAACTAATATTTCCCATTTTGTCTTTTATATAAAGAAACTCAGTGTGTGTTTGTGTAAATGTCTCT
TTTATTTCCTCAGATGAAAGGATCAGAAGAGAGCTGTACCCAAGCTATGTGCACACACAAGTTGGGCTGAGCCCAGAGT
GTTTGTTTCTCTGTTGTAATTATCCCAAGGCCTAGACCTGGAAGCTTCTAGCCTCCATACAACTTCATTTTCCAAGCTG
ACTGATTCAATCCAGCTTCTCTTGGCTTCTCACTGAATTTCTCTGTTTGACCTTGTACTAACTTTGGCAATATGTTCTA
ATCTTCTGGCTCCTTCTCATGTTCTGGCTAGTTCTGTCTTCACCTGTCTAGCTTGTTCTCTCTGCCACCTGTCTTTGTA
AAGCAGTCTTGCTAAAACTACCACACACAAACACCATCCACCTTTCTCTCTTGCTGCTCTTAAGTAGCCTCTCTTTCCT
GTGCTGGTCTTGTGTGAGCTGGGTGTATCCTATCTCCGACTCATTCTATCAAATCTCTCTCTGGTTCATCACTTTGTCT
GCCCCTCAATTAGACATTACTTCCTCAATTAGACGTTACTTTCAAACATAGCTGCTCCTGTCTACAAACTAACCTTACC
TTCATTTTGAGTGATTAAATGTGTACACTAAGATCTGTATTCCAACCAGATCACATAGACCTAGAAGGTCCTTGGATGT
GCTCCCTTGCCAGAGCAGCCATGTTGCTGGATTAAAATTCCTCTACAGGTGTGTAACAACAACAACTACTACTACTACA
ACAGCTACAACAGCAGCAGCAGCAGACCTGATTAGTATAAATTATTCACACACTGCAACAATTGGGGTGGAAGTTTCTG
CATATGTCAAGCAATGCAGATTTACCTGGTGTTTTGCTGAAGCAAGGCCCATGGGAGGACGCGTGATGTTTGGAGAGAG
TATAAATAGGACTCGGTGACAATGACGGTGCCCTTACATAGCTAGCTTTGCAATGCTTCATTGGTCTTGCGTCTTTATC
CTCGTTCATCTTCCCTTTGTTGAGAGAGACACAGCAGAGAACTTCTCCTGGTGTCCAGACTGGTCCTGTTCATTCCTCC
CTCTGGCTCATGCTGATTCTGTGGAGGCCTGGCTCTTTCTGTTGCTTTTTCTGTGCCACTGCTGCTGATTCATGTTTGG
TATCCTGACACTACTGAACTAGACTGCTGGTATCCTGACAAACGAAGATTGGAATCACCCCCCAGAAAACTACTTCTAA
ACAGGTCCACATCCCTTTATCCTATTAACCTAGAAGGGGGGTCAAAATGTTTGAGAATCCTTATTAGAAGTAGGTTTTT
TAAAAAAAAATCTAAGCCTACAGTTGTGGTCTAAAGTGAAGGTTTTGAGTATCAAGTTTCCAAAAGGTGTACTTCTGAT
GGTTAATTTAGATAGTCAACATAGTTGGATTAATAGATACCTGAGGTATTGAGATACACTTCTGGTTGTGTCTATGAGA
ACTTTCCTAGAGAGCACTGAAGAGAGATGGCCAGACCTGAATGAGGGCCTCTCAAGATTCGGGGTCCTAGAACAACTAA
AAAGAAAAAAAGGAGGAGGAGAAAGAAGAGAAAGAAGAGAAGGAGGAGGAAGAAGAGGAGGAAGAGGAAGAGGAAGACA
AGGAAGAGGGGGATGAATAGGAGAAAGAGGAGGAAGAAGAGGAAGAGAAGGGAGGAGAAGAGGGGGAAGAGGAAGAAGA
GGAGGAGGAAGAGGAGATCAGTTGAGCACTGGCATTTTCCCTCCACTGTTTTCTGGACCACTGAGAAGCTAAATGGTGA
GAGGTCACGGGTGGAGTTATGCTTTGCCTGTCATTCACATTTTGCCTGTCCTGGTAGACTCTATACCCTCAAACCATGA
ACCAAAAGAAATGAATTTTCTCTCAAGTTGTTTTGTTGTCAGGCATTTGGTCATAGCAACAAGAAAAATAAGTAATACA
CTAGTTTAAAAATCATGTTGGTCTAGGAAATCAATAACCCGTGTCAACTTATCATTAATATCCCATTAAAGGGGTAATA
TATTACTTTAAAATAACCCCAAACTTACAAACATAAGGCATTCTTCACAAATGTCTCATACTTTTGTAGCCAATGTATA
TAAGAGCAATAATTATTATTAAAATATGCATATTGATTCATTTTCAAAAAACTGTTTCTAGTCATTATTTTTGGTTTGT
ATTATGAAAAGCTGCAAAATAAATGTTTGTTCTTATTTTGCCAGATCATAAATAATTTTCCCTCTTTCTTTGCCATTTC
CAAAATAGATAACTTCACACAAGAGTGTAATACCAGGAAGGAGAAAAAAATCCCACTGACATTTTTAACTTTGTTTATT
GAATATCTGGCCAACATTTCTTTTGAACTTCTCATCAGTCTGGGTTTTTAAACTCAGTGCTGAAACACTTTCTGGCATG
ATCAAGCCTAATTTTATTTATAGCTAGCTGAGATTGTTTTTGACATATATAAAATATTCAAAATATTCTTATTTCAGAT
TCAGTTCAAACATTTAGGCCGCAAGTGGACAATATTTTTCAAACTAATTTGGAAGTCCTCGAACATATGAAAATATCAG
ATGAAAATATCATATCTCTTACGCTACATGAGTTACTGAAACTTTCAACAGTACCTATAATTTTTCTAACATTTAGATC
AATCATTTATGATATTATGAAGAAGGGAATCTACTCTACAGAAATCTTCAGTTAAGAAGATTAATTTTGGCCGGGCAGT
AGTGGTACACACCTTTAATCCCAGCACTTAGGAGGCAGAGACAGGCGGATTTCTGAGTTCGAGGCCAGCCTGGTCTACA
AAGTGAGTTCCAGGACAGCCAGGGCTACACAGAGAAACCCTGTCTCGAAAAGAAGAAGAAGAAATCTTTTCTAGTCAAA
ACTACAACTTAGATCTTGGGAAATATATTATATATTTAATTCTAATTTCAGGATAATTCCACTACTTCTTATTACCTGA
AATTAGTTTGTTAGCAAGTTCAGCATTAGTTGAAAATATCTACAATTTTTGTCTCATTTGGTTTTGTTTGTTTGTATGA
TTGTAGTTTTTTAAGACAAGGTTTCTCTGTGTAGGCCTGGCTGTCCTGGAACTTACTCTGTAGACCAGGCTAACCTCAA
ACTATCAGATCTGCTTGCCTCTGCCTCCTGAGAGCTGGAATCAAATATTTCTCATACACAGGAATTTCGTTTTTGTATG
CTGTAGCAAGTTTTAGTTGCTCTCAGTATGACATTTGTACATAACTTCCTCTAATTTTTTAAATTGTGTGTGTGGTGTG
TGTGTGTGTGTGTGTGTGTGTGTGTGTGTCTGTGTGTGTGTAACAGAGAGGTCAGTCAACATTGGATGTCTTCCATGAT
CAACTTCCATTTTACTTTTAAAACAGGTTTCTCACTGAACCTGAAGCTCACCAATTTGGCCAGACTGGTTGTCCAGCAA
GCACCAGGAATCTACCTGTCTCCAACCCCTCCACGCTGGGTTTACAGGCATCCGTAGACCATGCCCATCTTTTTGTGGG
TGTTAGGATCAAACCCAGGTCCTCACGCTTGCGCAGCCAGCACTGTACAGACCGAGCCATCTCTCCTCCCCTGGTTTCT
TTTTCTTTTGTTCTGGTGATAGTACTCTTGTCCCCCTAGATTCTCATTCATACTCTCATTTTATACTTAAAGCTTTTTG
CACTCATTCGCTCCTTAATTAATTAACTACTGTGTGTGGTGTGTGTGAGAGCGTGTGTGTTGTATGCGCAGTTACATGT
GCCTATGTGTGTCATCTGAAGTCAGAGAATGAGGTCAGGTGTCCCGCTGTCACTCTCCATCTTGAGATTTTTGAGGCAG
GGTCTCTCACTGAGAGACCAGGCTGGCTGAAAACAAGCCCGAGTGAGCCTCCTGTCTCTAAACGAACACAGTGAAGGAT
TTCCAGGCTCAAGTGACCACATCTGGCTTTTCTTTTCTTTTCTTTTCTTTTTTCTTTTTCATTCTTTTTCTTTTTCTTT
TTTTTTTTTTTTTTTTTTTTTTTTTTTTTTTTTTGAGACAGGGTTTCTCTGTATAGCCCTGGCTGTCCTGTCCTGGAAC
TCCCTTTGTAGACCAGGCTGGCCTCGAACTCAGAAATCTGCCTACCTCTATCTCCTCTGCCTCCCAAGTGCTGGGATTA
AAGGTGTGCACCACCACCACTCAACCACACCTGGCTTTTTGATGTGGTTTCTCAAGCTTGCCCACAGAGCAAGCTTATT
ATCCATTAAGCCATTTCCACGGTCCCTAATCTGTTACTTTCTAAACTAGAAAAATAATTTCATTAGTAATTACAACTTA
TATGAGTCCCACTGTACCTATATTATCTGAATATAATGTTGCACCATTATACTGATGTATAAATATATAAATAAATTCA
CGCTGAGTCAATCCATTAGCACTGACTAGAAACACGAAGTGCTTATGCATAATTATAGAAAACCCTGTGACACAAGGAT
ATAATGTTACGTAATGCAATATAATTTGTTAGAGCAAATTATAGTCCATAAAAATTATAAAGTTGTGGTTTTTTTTTAA
AGTTGTGTTTTAAAAAGAAAATGTCTTATACTGTTTTGGTGTTTAAGTTGTGATATCGCTATTTCATATTGGTGTGGAA
AACACACTCTTAGGAAAACAGTCCAGATGCTTCTTCAAGTTAGCATGTGACAGAGAAGTCTAACTCCTGGGCACTCATG
CTACTGGCCTGAAGGTGTATATGTATATGTATATGTATATGTATATGTATATGTATATGTACATATATATGTTAACGCC
AAGATTGGTACATGTATATTGATGGCAGTTTTATTTATAATTTCCACAAATTGAAATAATAAAACTGTATCATAATTGA
CAAATAATAAAATATGACAAATAGCATATCTAAATAATGAATTTCTGCTTACAATATAAAGAAATAAACAATTTAGTAA
TTCCACAAACTGGACACAGCTTAACTAATTTTTCTAAGTGAGATAGAATAGATCCCAAAGGCTATGTATTGAATGCTCC
CATTTGTATGACCTTCTGGGAAGCACAAAGCCGTAGGAACAGAAAGCAGGTCAGTAGTGTGTGAGTCTGGAGAGGAGGG
ATATGTTCAGTGTTATGAGCTAGAGAAGGGACTCCCAGGACGGTTCTTTGTGATGCTGTAATAGTGGGTAAATAACTCA
AAATCTGTAGGAATTAGTACAATAAATAGCTTTACTGTATGAAACGTCATGTGAGACTAGGTTATCAAGTGAACCCAGG
GTGGGATGCAGGCAGATAGGTGAATCCAGCTCTATTATAATCAGTGGCATAACTACTTCCTATTTTATTTCCCTGTTAC
TGTGATAAAAATACTCTGATAAAAGCAATGTAAGTGGTAAAAAAAAGTTTGTATTGGCTAGTTTTTTTCTCACCTTGAG
CCAAGATGGGTCACGAGGAAGAGGGGTCCCAGGAGAAAAAATGGTTCCATCATATTGACCTGTAGGCAAGTCTGTGTGG
GCATTTTCTTGGTTTTCTTGATGTGGGAAAGTCCAACCCACTGTGGTCAGGCCACCCCTGGACAGGTGGTCAAAAAAAA
AAAAAAAAAAAAAAAAAAAAAAAAAAAGCAAAGCAAAGCAAAAGAACAATAAGAATCTAGGATCAGCAAACTGTGAAGA
GCTAGCCAGTAGTCAATATTCTTCCATGGCCTCTGCTTCAGTTTGTGCCGCCAGGTTCCTGCCCCGACTTCCCTCAACG
ATGGACTGTAATCTGCAAGGTGAAATAAATCCTGTCCTTCTAAAGTTGCTTTTGGTGATGGTGTTTATTACAGCAATAG
AAACCTAACCAGAATGGGCTTCATTTTAGCTCACGATTCCAAGAAAAGTATCCCAAAGGCCAAACAAAAACAAAACCAA
AAACAAAACAACAACAACAACAAAAAACCCTCACTCATAGGAAGCTCTAGCTTGTGAAGATTCAAATTCATTTAAAGCA
AGAAAGCCCAACTTCAAAGAGTTCCATGCTCCTTGAAAGCAACTGCCTAGGAAAATGTCTCCCCAAGGCCAGCACACAA
AAGCCAATGTGGCCGTGTAGTTCAAGGGTACCAGGATCTATCCCAAGCTAGTAGCAGAATCTACAGCGTGGAGAATATA
AGATGTAGAGACTCTTGATGAGCAGCGGAGGCCCGGCAGTGTGTAGCAGGGCCGGAGACCCTAAATAGAGGTACTAAAA
GGCCATTGTATAGAGTTGTGAAAATTAAGCCCCAGTGTTATGGAGGCCTCCTAGGGTATCAGAGGTCAGAACTAAGGGA
CATCTGTCAAGGAGATCTGCAGGCATGGAGAGCAGGTGGCCTGGGGGAGACTCTGTGTGTTCTACAGGTGACAGAGTCA
GAGGAGTGAAGCGATGGAGCCCTTTGATGCCCAGTTGACTACGTTACCAGCTCCAGATGCTGGATAGGAAGCTGCAGGA
TCTCCTATTCATCCTGCTGGATTTTAGTTTTGTTTTGGTGTAATTGTACTCTGCTATGCCCTGGTTCTCCCATTTTTAA
ATATATTTACTCTGTGCCATTATATATTGGAAGTATGAACATTCTTATCATTATTATTTTATAGGAGCTGACAGTTAAA
AGACTTTGGACTTTTATTTATTTTTTTAAATTTATCATATGCAAGTACAATGTCTTCAGACACTCCAGAAGATGGCATC
AGATTTTGTTATGGATGGCTGTGAGCCACCATGTGGTTGCTGGGATTTGAACTCAGGACCTTTGGAAGAGCAGTCGGCG
CTCTTAACCGCTGAGCCATCTCACCAGCCCCGACTTTGGACTTTTAAAATACTGAAGTTATTAAAGACTATGGGGACCT
TTAGTGTGGGGATGAGTGCATCTTTCATGGTCTTGAGCCTGTGAGGAAGGGAAATGGAAGGGCATATTAGTTATTTTCC
TGTTGCTGTGATAAAACACCATAACCAGACAGACCTGACTCACAAAAAGAAAGAGCTAACTTTGGCCTTCGGTTCCAGA
GGAAGAGCCCATAATGACAGAGGAGACATGCAAGGCAGCTGATGGCCGCAGCTGTGGAGAAGGTCACATCTTCAACCAC
AAGCAGAGAGCAGAAAACCAACTAGAAATGGGACAAGGCTATAAACTCTGAAAGCCTGGGACATCTGACATACTCCTTC
CAGCAAGGCTCCATCATCCAATGACTCCATAATTTCCCAACAGCTCCTCCAACCAGGGACCGAACCCCAACTGTGTGAG
ACTGTGGGGGACATTGCTCACTCAAACCACCACAAAGGTTATGGTTTCAAAGTGATGTGTTTGATCCGCTTCCCCCAAG
GCTTACGGAAACCTCGTGGAAGAGGAAGGAGAAAGACTGTAAGGTCCAGAGGCCAAAAAGTGTCTTCTGTATATGTCAG
GACCACAGAATTTATAAGCTCAGAGCAGAGCAGCTGTGGTAGTCCACACAAGACTCACACAAGAGAAACGAACAAATAA
TATCAATATATCAAAAGTGCATTGTTTAGGATGATCAAGGCAACCAATAGACCTCTAGATAGAATACTGGGGGCAGGGA
AGAAGACAGCTATAACCTGCCAAAATCTGGAATAGGAGACATATCACTAGACAGTTTATAGAATTAAAAGATTAATAAG
GCAACATTACAAATAGTCTCTCTCACACACAAGTTGTGTAGCTTAAGTGGAAAAATTCCTTTAAAGACACTGACTGTTG
GTGATCCATCAAGAAAGAGAACAGAGAACTCAAAAAGTCTAATATTTACTGAAGAAACTGAATTGGTATTTTTAAATCT
CCCCATAAAGAAAACCAAGAATCAACGGCTTCATTCAATAATTCCATCCAACTTAAAATTGTTTCACTAACAAATTATC
TTAGCTGAGCATAGTTGCTCACACTTTTGATCCCAGCACTCGGGAGGCAAACACACGTGGATCTCTGAATCCGAGGCCT
GCCTGGTCTATGGAGGACGGCCAGGTCTACACAGAGAAATCCTGTCTTGGAAAACCAAAACTAACAATAATTTTTAAAA
GTACATAAAAAGTTTAAATATTTACGTGTGTCTATGTGAGTATATACCTGTGAGTGCCCATGGAGACCAGAAGAGGGAA
TCAGAGACCACTGGTTCTGGTCATGAGCTGCCTGATGTGGGTGCTCAGAACAGAACTCTGGTCTTTCTGAAGCATGGGA
AGTGCTCCTAACCACTGAGTCATCTCTCCAGCCCTTCCGTTCAACATTTAAGAAACAATTCCAATTTTACATAAGCTCC
TGCCACAAATGGGTGATGCCCGTGTACTTCCTGGCTCATTTTCTGAGGCCAGCATGACTGTTATCCAAACCAAATGATG
ATATTAATTTTTGTTGTTGGTTTTGTTTTGTTTTTGCTTTTATTGGATATTTTCTTTATTTACATTTCAAATGTTATCT
CCTTTCCTGGTTTCCCCTCTGAAAAACACCCTCTTCCTTATCCCCTCCCCCTGCTCACCAACCCACCCACACCTGCTTC
CTGGCCCTGGCATTCCCCTACACTGGGGCATTGAGCCTTCACAGGACCAAGGGCCTCTCCTCCCATTGATGTCTTACTA
GGCCATCCTCTGCTACATGTGCAGCTGGAGCCATGAGTCCCACCATGTGTACCCTTTGGTTGGTGGTTTAGTCCCAGGG
AGCTCTAAGGGTACTGGTTAGTTCATATTGTTGTTCCTCCTATGGAGCTGCAAACCCTGTCAGCTTCTTAGGTCCTTTC
TCTAGCTCCTTCATTGGGGACCCTGTGCTCGGTCCAATGGATGGCTGTGAGCATCCACTTCTGTATTTGTCAGGCACTG
AAAATGATGATATTATAAGAGAAGAAAATTGCAGGACAACATCACTCACAATCGTAGACAAAAAAAAAGTGCCAACATT
TTAGAATGCCAAATTTAATATATATAAAAGAACAACTTATTATCATTGGGTAAGGTTTATCCAAAGAATGCAAAGAATG
CAGGCATCTGTTGTGGGTGAGGGAAGGAAGAAACTACTGTGGTCCACACCATGGCACACCTCTCAGTAGTAATGGAGTA
GACTACTGATACAAACAGGAACTCAGAATCTGTCCATGGAGTTATCTCAGGTGAGAAGAAAAAAAAAAAAAAAAACCCA
GCCCCCCCCCCCCCCCCGCCCCATGTTCCGTACAATATAATTTCGTTCAGATTCTTGAGTCTAAGGAAATGAAATAGTT
CAGTGATTGAGAGTGAGGAAGGGATAGGTGTGAGGAAGGCAGGAGCAGGAGTGTTCCCATGGTGATGGGACTCTTTGTA
CTTGATTGTCTATATACGGGCTCGACAATGAACTTGATACACAAGATACTACCATGGTGGAAGTGGGTAGGGCAGGTTA
GATAAAGTACCCACGGGTCTGACTTTTTTTGCAACTTCTTGTGAACCCATAGTGACTTCCAAATGAAGAGAAATCTGTA
GTTATGTTTTTATGTAAATAGCTGTTGGGAGCCGCCCCCACATTCGCCGTCACAAGATGGCGCTGACATCCTGTGTTCT
AAGTGGTAAACAAATAATCTGCGCATGTGCCAAGGGTATTTTACGACTACTTGTACTCTGCCTTTCCCGTGAACGTCAG
CTCGGCCATGGGCTGCAGCCAATCAGGGAGTGATGCGTCCTAAGCGAAGAATAACTCCTCCTTAAAGGGGACGGGGTTT
TCCGCCATTCTCTCTCTCTCTCTCTCTCTCTCTCTCTCTCTCTCTCTCTCTCTCTCTCTCTCTCTCTCGCTGGCATCTC
TCTGGCTCTGCGCGCCCTGCGCTCGCTCGCTGCCTAAAGATGTAAACAATAGAGTGCGCTCTGCGCTGGCTCCTAAAGA
TGTAAATAGAAGGGGCTTTCGCTTTTGGGGCGGAGGGTTTGCGCTCCTAGCTCCTGAAGATGTAAGCAATAAAGTTTTG
CCGCAGAAGATTCTGGTCTGTTGTGTTCTTCCTGGCCGGGCGTGAGAACGCGATTAAGAAATAGCCAAGGGCAAATAGC
CAAGCCCTTCTTGAAGAACATGAAGAAACATGGGTGGTGGGGTAGCTATATGTTCCGACAGCGATCTTTGGTGTAAAGA
TAACAACATACATCAGGTTTTAGCCCTTGATGTATTTCCTCTCTGCAGAGCTTTGGAACCTTTCCGCAGGTGCACCAGG
AGACAGGTAGAGAACAGTCCTATCTGCACCATTTGCAAAGCAGAAAGAAGGCTGGGTGCTCTTTCCATGGTTAATTTCC
TGGCTCTGCTAAGGTACCGTGGTTATGTAAGATGTTATCGTGGGGGCTGTTGGATGAAGAGACACAGAAACTTCCTGTA
CTATTTTTTGAAACCTCTATGTAGTCTAAAACTATTTCAGGCTTTAAAAACTGGACACATTTTTACTGAAAATGGTTTC
AAAATTGGGGCAGACGATGGAATGCTAAGCAGACACGGGAACCAAAGGCTCTGCTCTATGCATCAACAGGAATCTTGGG
AGTACAGCGAGAAGGGAAGAGCAAGTCAGAAGACAGGGTTTGAAATATGAATTCAAAAGACTCAAGAGAAATGAAAGTG
TTGAGAGTTTGGGTTTTCTGCCTTGGGGAAGGAGGAAAAGGGGGAGGGGATAAAAGAAGAATTAGAAGACAGGTGTGTG
TTCACTGCGATATGATGTCTTTCCATGTTCCTCATCCAGAGAACACACTCATCAGGAAGGGCAGAAAGATGCCATGCAG
AAGAAACTCTTTCTAGTGAGACTACCAAAAAGAGGCTTATAGTGGCTCTTTTTATCTATCACATGGGAAATGCCTGATA
TCCCAGGACAGAATAAAGATGATCCCTTCCCCTAAGAGACAAAGGAATGGCAAGGGTAAGGGAGAGACTTGGAAGAGAG
CAGAAGCATTCTAAGGTTAGGATTCACATACAGCATGAAGACCATGCTGTGTAGCTCTGCCCAGACTGCAGGAGCCAAC
CCAGCCGACTGAACCAGTACAGATGAGGAAATGCTGGGTGGAAGGTGTGACACCCCCCAACCCCCTGAATGCCTTGACC
TCCACAGGCAGGGAGGGGTGTGGCTTAGGAGTAGTTCTTTCACTGCTGGAGTGTCCAACTTCTAGCTACGGAGACACAA
CATTGCAATCTACAAACTTCACAATGTAAAACCAGGCAATGGGATCAACAACAATAAACAAAAACTTGCCCCAAAAAAT
GTAGAAACCCATCCAGTATCTCATAATGTTCTACATAAGATTACAATATTGTGTTGTGCTGAATTCAAAGCTATCCTGC
AAGGCATATGGCCTGTGGACAGTGGGTCGGACACACCTGCCAAAACTTTCCTCAGCCCATGCTGGATCCCGAGACAGCA
ACACACTCGGGGCAAAAGTAGGGTGGAGTCTGAGTTATCTTAGTCTGGTGCCCAATCTGGCATGCTTATATGCTCAGAG
GGATGACCTCCGACTCCAGGGACTTCAAACCGAAGAAGACCACAAGAATAGAACAGTTACTCTGATTTGAGAGTAGTTT
TAGAGGTTAATTCGAGCATTCAGCAACTGTTTCCTAAACTCTAGGTGAAGTGGAACCTCAGCTGCTTCCATGGAGTTTC
CTGTGAGGGCTCAATGTGCGGTGGGAGACTCTAGTGGGTACATTCGGGGTAAATTCATAAGTGTCCCCTGGGAAATCCG
TAGGCACGGGTGTGTGTGTGTGTGTGTGTGTGTGTGTGCATTTCACTCCTGGCTTGCTTTGTGTAACATACACTACACA
TTCCAAACAGTCCCAAACAAACATTCCCTGAATGGCCAGAGAAGGTCAAGACCAGCAAGTGGCTGGAGAGGAAGAGGGG
AGGGGCTTTGTAAAGCCTCAATTAACCCGATGCCTTAGAGTGTACTGCTTGGGGACTGCGTTTAACCTTAGTCTGGCGT
CTACTCCTTGCTCTCACGCTACTGTGTGTGCCCTGCAAACTTAGAGGTCGCTCTCTTATACTCTTGTAGCTCATTACCC
GAATAAACCTAACTAGCAGGGCGCAGGAGTGTGGTTGGGCTGACGGTCTCAGGATCAAAAGAGGCAGGGCATTGTCTTT
GGGTTAGTGGAGCTGTCTTCTACCAGGCAGGTTAGGTTTCAAACCCTAGTAGGCATATCCTGTAAGGGTGTCTTCTCTC
TCACTGGCCAGCTCCTCTGGGGACAACCACAGATCCCCTCCGCATGAAGAGTCTGAGCCTAGCTAGATAGGAGGTAAGC
TAGAGGCCAGCAGGGGCCACCGAGGAGCCATACCCCAGGTCTGAGCCAGGTATCCGCACATCTTCCTACTCACCACCAC
ACCACACACTGACCCTTTCCTATGTTCCCGCCCTGTCTGACCATCTATCTGGGTCTGAGACGGAAGAAGGGAACCGCAT
TTAGGTGTCAAAGACCAGAAACAAATGTCCTCTTGACCAAGTCCTGTCTTTTTTTTTTCCCCCTAGGTGGGTGGGTGAG
ACAGAAGCAGCAGGGCAGAGCAAGACGCTCCAAAGAGGGCACCTCTCTTAGGCTCAAGAGGAGGTGGCTCTCCGGAGCC
GGCTGCCCGGGTGGGGGCTGGATGGCGCACGCCCCCCAAGCCCCGCAAACCCACGGGCTCCGCATGACATCAGAGGCTT
GGGAGCGCGCAGAGCGCAGGCGCGGGCGGAGCAGGCGGGCGGGAGGGGGCGCGTGGCGCTGCTCGGAGAGCGCTCCTCC
GCCCAGCTCGGTCCCTGCGCCCCGCTCCGCCCGTGCCTCGCTGCCCGCGCCGAGCTTCGGGCGTCCACAGCGCGCCCGT
CGCCCGTGCAGCCAACGCCACCCAGGGCTGCGCCGCGCACCCCGCCTCCTCCCTTTCCGCCGTCGCCGCCGTCGGACAC
GCAGCCGCAGGCCGGGGCCCGGCCGCAGCTGGGGAGTCAGGGGCGCGCGCAGCCAGCCCTTCCCCCTCCGGCTCCCGCA
CCGCTCGCCGCCTCCCCTCGCCCTCCTCCTCTCCCCTCCCTGCTCCTTCACCGCTTCCTCCTCCTCCTCCTCCTCCTCG
CCCGGCCCCAGCTGCCAGCACCATGTCCGCAGGGGGAGATTTTGGGAACCCGCTGCGAAAATTCAAGTTGGTTTTCCTA
GGGGAGCAGAGCGGTGAGTACTCGGCTTATTCTCTCGGCTCGGAGCCGAGTCTTTTCCCCACGCCGCCCATTCAGCCAC
CCCACTAGGGACCCTCCCAGCCCTGGCTCGCCAAGGCCCTTCCACGCCCCGGTAGAGGAGAGGGATGCAGATGGGGAGA
TGGGGCTGGCTCGGGTGGGGCCGGGCTGGGGAGGACCAAGGGGGAAAGGAAGAAGGTGGTTCTGGGGGTCAAGGGATGA
GGGTGAGTGTGAGATCTGGGTACGGGTACCCGGTGCAGGTGCGTCCGGGAGGACCGAGGGCGGCGGCTGGCTCACCCGC
TCTAGGCGCCGGGCTCGCGGGCTCGCTGACTAGCTCGCACTGCGGCACAGGCTGCGGAGCACTGTGCAGGTGTCTGGGT
GCCGGCGAGGGTGTGGGCGAAGGGACAGGAGCCTCTCTTGGGAGAACGAGGTGGGGCTGGGAGCTGGTTGCCCCCACCC
CAAGGTTGCGCCCGAGGGGCTCCCTGCGGACCCTGCCTGGAATCTAGGGAAAGGACGCGTCCAGCTCTGGAGCAGATTG
CTGCTGGGAAATGGGGAACACGACACCTGTCCCTTTTCGAAAAGAGTGGGTGCAAGTGGTAGGGTAACTGGGCACTTAG
CTTCTCTGGCTTACATCCGACTGGGCAGAAGGTTCTAGACTCACTGTCCTTCACTAATGTGTTAGCCTCTTTGGGGCTT
AGGGGTGTGGATCGTGAGAGTGAAGATTCTTCCCATAGCAGGTGACTTGGGGGATGGTACAGAGGCGTCTCATATCTGT
GAGCTTGGCACTGGGCAGAGAAAGGCCTAGGCGACACGCCCTGCCTGGCTTCCCTGCCCCCTCTAGAGAGCATCAGTGG
AGCCGGTGGATTCTCCCACCCCCCATCTCCCAGCTCACCGCCCTTCTTGGGGGAGGGGAGGTCACCGTGGCAGCCAGCC
AGAGCCTAAGGAGGGGGAGAGTAAGGGTTTCTTTGCTGCCCAGGAGTCTCCCCTCCCAAGTATCTGACACCCAGGTTGG
TCCAAGGTGGGAAGCTCAGACAGAAGAGCAGCGACCCCTCTGATACCTGTGGGAAGGAGTACCTCCTTGAGAGCCTCGG
TCCAGGAGTCACATAACCAGGGGGTTACAGACATAAGCTGGGCTTCTGAGCATTTCTGCTGGAAGAGAGGGGGATCCCG
GGGGGGGGGGGGGCGGGATTCCCAGCTCTAGCTCTGTAAACCAGTTCTGTTAGCTGGTTTGGAGGCCAAGAGGAGGAAG
GCCGTGGGAGGACCTGGTGGGCCCAGGCACCGTGCAGCTATGACGTGTGCGTGTGGGCTGTGGAGGTCACATGGCCACC
TCCTGTCTGCTGATGCTCAGTACCTGAGCTGGAGGGTTGGGAGCCGGGTGGAAGTCGGGGTTGCCTGTCCCGGAGAGCT
GGGTTTGGGACATTTCACATCGTACCCAGCTCAGCTAGGTAGCCAGCTGGATCTGATTCCCTGTGCTTTGTAGATCCCA
AGTGTCACAAAGGACACCTCCGTTCCTTGCTGTGTGGAAAGGATTTTGGGAGCACCTCAGCCCTAGGAGGGAGCCTGCA
GGTGCACAGCAGCCTTGGGGGGTGGGGGGGGCTCTCAACTGTGTTGTCATTTGCCTGATTCCAGGCCTCCTGGACACTC
AGTTTCTGTCTCTGAGTAGTGGGTAGCTGGAAGCAGCCCTTATGCCTCATGTTTCCCCCAGTGCCTGGACTATGCTGGA
GTTCAAAGAATATTTGTTAATTCTAAATCTGAACACAGTTCCATAAAGAATCATATGTGGTGAGTTTGAATGAAACTCT
GGGCTCCCTTAGCAACTGCTGATGGGTGGAACACACACACACACACACACACACACACACACGCCACCCCCAGTGAGAG
TTCTGCCAACCCCTCCAGGTCTGGAGTCACAGAAGTGAGAGACAGCCTTTCCTCACATGCTCATGTGGAGAGAGAGTGG
GGAGGTCTTGCCCAGAGACCCACTGTGAGTTGTGAACACTTTGGGAGCAGGAACCCCCACTTCCATTTTCCCGTCTGCC
ATTTTTAACTAGAGTGTCATTGGGAAATAAGATGACCTTCAATGCGGAAAGCCAAGTCTGGAACCTCTTTGAGAGGATT
TATGGGCTCAGTTAGTTGGTTGTGGCCTGGTGGAATGCTTGGCTAATTGGTTAGGGGCAGGATTCATAGGCCCTTGGAA
CATAATCCATCATTGTGTTATTAGTGAGTTGTTTACCGCGGGTGTACAGAGCTTGTGGTGTCTGTGAGTCTCAGAAGTT
ATGTGTATTCCGGAGCCTAGTAGGCCGTGGCTGTAACCCCTTCCATCTTACCTCCTGACAGTTTGCATGCCAGCATCCT
CTGCAGCCTGTGCTGTGGAGGGCGGCTCCTGACATCTGGTCCAAACCCTACCCAAGCTCTCCCTTCCTGTGTGATCACG
GAGACCCCTCACGTTCCATACCAGGGCACTTGAGCCAACTGGAATCCGAGGCCTAAGCTTCCCTATAGAGGCCAGCTAA
TGGAGATGGGGGAGGGAGGCGATAAAAGAGGTTGAAAGGACACAACCTTCTCCTGATCTCTATTCCTGACATTGTGGTA
GCATCCCTCTAGGCTTGTGGACAGAGACAGCTGGATTCTAAAGAGGGGTACCAGGCTTGGAGCAGGCTAAGGTAGGAGA
TACCCTGGAACAGATAATCATAGGTAGGCATGAGGCCTTCAAAGGTACCGAGGAACTGGTTCAGGTAGGAAGCCTTTGA
GTGCCTGCTCTCCGGAACGCGCCTCTCGCCTGTGGACTGTAGTCTGACTGGCAGTGCCTGTGTCACATGGAGTAGGATC
CTGGCCAGGTGCTGTGGACTTAGGGTGAGGTGTTTCCTTGCGTCAGGTAGGTCATGCAATGCTGCTGGCCTTCAGGTCG
CTGTGTGGCGCCTCCCGATTAGCCCTAACACGGGTGGCAGTGGTGGCGAACCCAAGAGGGTGGTGCACGCCAGACTGGT
GTGGAGCATCTGAAAAAGACAGGCAAAGTCTGAGGGAGAGTGGGCCTGCGAGGTGTCCATCTTTCTCCCAAGTTATCAG
AGCCTCCCAGGGGATCTGGACCTTACCAGACACATCCTGGTGTGTGTGGTGAGGAGGGAGGGAAGGAAGAGTGGACAGA
CATGGGGGGAGGGGGGCTGTCTGCTTTCCCGGGCTGAGGCAGTAGGCCATCCGTCTCTATAGGCCTGAGTACAAGGAGT
AGGGGTAGATTTCAGAGAAACCCCTCTCTCCTTGGCCCAGCTCTGCAGAAAGTGTAGGTAGAGGCACGCCTGTCAGGAG
CGACTCTAACCTTCCGAACGCTACAGCCCTCTAATGCAGTTTCTCGTGTCGTGGTGACTCCCAGCCATAAACGCGTTTT
GTTGTTGCTTCATAACTGTAATTTTGCTACTGTTATGAAGAGTAATGTCAATATCTGATATACAGGATAGCTGATACGT
GATCCCAAGGAGGTCTCGACTCACAGATTTGAGAATGATGGGATTGCTGGCACCCGGTTGAGACCGTCCTATACATCCT
CTTCAGCTAAGGGTTAAGAAACAACTGTCCACAGACTGAGCCCTGTTTCCTGCAACCACCCCATGCAGCACAAACCGTC
CTGTCCTAGCTTGGTGGCAAGCCCACTGTCCCTTTTAGAAGCTTTTCTCCTTTCAACTTCCAGGCTTGTCACATGGACA
CAGCTTTGATTCTTTTAATGAACATTAAAAAAAAAAACCCAAAACCCCACAAAACTGGTTATGTGCAGTCCCATGACGC
CTCTTCTGTCTCAGCTAGGTAAGGATTTTAATTTAAGGATTTTAGGGTGAGTTTTAAGCACCCTAAGTGGGTGACCAAA
GCACGTTGAGAGGATTGTGTCTGAGTCATCTCGCTCCCTGCTCCCGTCTGTGGGCTGTGCGTGCTCACCGAGGATCTTG
ATTCGAAAACGATGACATCTTTGATGTGAGAGATTTGCTGATGGCCTGTCAGCAGATTAACAGTGAAGGGAGGTGAGGC
AGCTTTGTGTGGTGTTCCTGGCACCTGGCACTCTCTCAGGCCAGATTCTTCTTATCGGGGAGTTGACAGTTGCTTTGTC
TTGTTTTGTTTTTCCAGAGAATTGCATCATTATGCCATGGTCATGCCCAAGCACCTGAGATTCTCAAAGGGTTCCCCAA
CTCCCTTAATATCTCTGAAGAAATATGGAGGTGGAGTAGGGGCTCCTGTACTACCAGAGGTGGGGAAGGACACGGCAGA
CCAGACCAGATCTCTTGGAGGCTCAGACTCAATCCCCATGGTGTTCTGTGCCTTGCTTTCTCATCTCGGTTTTTTTCCC
TCACCTTCTAATTTCTCTCTCCCATCCCTCTCTCTTGGGTGCTAGTACAAGAGAGAGAACCTTGGGCGTTGCGTATAGA
CCTACAACCCTTGATTTTTTTTCCCTATGTTTAACAAAGTGCGTCGGAATGACACAGACAGTAAGATGAGGCTGTCTGT
ATTCCTTCTGTTACTATAATGAAACACCCGAGTCAGGCCAGCTTTATAAGGAGAGCAGATTTGTGGAGCTCACACTCTG
GAGGGTCAAGGACAGGGTGCTGGCATTGGCTCCATTCTGGTTGAGGCTCTCATAACAGATGACATCATGGTTGGGGAGT
GATTCTGAGGGCACGAGGTCATATTACCACAGGAAGTTAGATAACTTCACAATCCTTCCCCAACACTGGCTCCAAGGGA
TCGAACCCCTCTTAGCAGGTTTTCAGGCTTACTCTTTGGGGGAGGGGTTAGGTGAAGGTCAGGGGCTAGGAAGATGGAC
AAGGGTCTTACACAGGCCTCACTGGCTTTGAACTCCCTGTGTGAATGGGGATAACCTTGAGTTCTCCTCCTCCTCCACC
TCCTGAGTGCCAGGGATTGAATCTAGGGCCTCATGCATGCCAGATGAAGCACCAAATCCCCTGGTCCCGCCTCTTAAAG
GACCCGCTGCCACATGAGGACTGAGCCACATCCAGACCAGAGACTTAAAGGTTAACCCTCGAGAGAAAAATGTTGTTAT
TTATAATCCTCAAATGCTCATAAATCATTTTAAAATAGAACAAAATAACTTCCTGTCCTTTGCTTCCATAGTGCACCCC
TAAAACCAGTCTGTGTTGCCCTGACATTGCAGACCATCCTTAACAGACCTGAGAGAAGCGGCCTGTAGGTTACCTCCAC
CTGGCTCTCCCCACCCAACTTCCGCACCCAGTGCCTTTCACTGTGGCTTGACACAACAGGGCTCCTTGGTCTCAATATG
ATGACATTGTTGGCCACAACTGTCTGGGGAGCTGAAAGGTTTCGTAGAACAGCAAAACATAAACAGGATTGAACTACTG
TGCTGGACACTGTCTGTCTGTCTGTCTGTCTGTCTGTCTGTGGTCCTTTGTTGTTTCTTGCTGTGCAGAAGAGATGATG
GAGGAGCCCTTGCTGGAGGTTGCCAAGGAGAAGGACCAGACACGCCCTGAGATGGGCAGCTCTTTTGTTCGAGCCAGAA
AGACAAGCTGCTGGGAAACAGCTCCAGGCAGGGCCTGGGACTTGAAGTCCCAAGGGGACTGGGAATGGCAGGCGCTTTG
TTTCTAGAGCCAGGGAGCCTCCCCCCCCCCCCCCATGTTGGCTTACTTCGAAGGGCTCCTGAGGAGGTCTGTATTATAG
GTCTGGGCTTCAGAAATGAAATTTTCTTCATTATTTCTATAACTTCTGCTTGAAACTTAAGAGCGATCATTTTAAAGAG
TAAAATTTTTGTTGAATACTTCTTATCACTTTAATAAAGTAGCTTATTTTTAGAAGAAAAACATATTTATCCTGTTCTC
TTGGTAACGAGACCAGTGGGAGTCCAGGTAACTGGGGCTACGTACCTCGGCCTAGCATCGTATCCAGGGAGTGATGTAC
TCAAGGGACAGGCCTGGGTCCTGAAGAGTACAGCTGACAGTGTGGCCCTGAAGGCAGAGTTGATTCTTTAAAGTGGTCT
CCGAGAAATTCACTGAAGGGCTGGTTGCCCGTGTCCTTCCTCTGTGAACCTCAGGTCAATGCCCCGGGCTCTCCAGGGG
CACTTTGTACCTTCCTGGTCACTCTGTGATAATCATGCACTGATTGCTATTTTGCGGTTCGTGAGGCTGCGGCTTGGAA
AGAGCTGGTCCTGCCTACCATTAGTGTAACAAATTAGAGCTTGGAGTGCCGGGGTTTGGGGGACAGCTGAGTCAGGAGA
GTGCCTGCCTTGCCAGCATGAATAGCTAGCCGAGCTTGAGCCCCAAAACTCACAAAAAAAAGTCGAGTGTGGTGGCAGA
TGCTTTTAATCCCAGCACTGGGGTGGCAGAGGCAGGTGTATCCCTGTGGCTTACCAGCCAGCCAACCTGGTCTACGAGG
TGAGCTCAAGGCCTCCGGGTCAATGAGAGACGCTGTTTCATGAACCTTTGGATCAGGTCATTATCCAAGGCAGGGCCAG
GAGTGGGGACAGAAGAATATTGAGGACTTAAAAGCAGAGTCTGCCATGAATGAATGACTCAGAACACCTGTACCAAACA
CTTTGCTAAAGAGGAACCAGTAGATCACCGTGTAGCTCCACTGACCATGAAGGATGCTGGAGCCAACCACAGGGAGGAC
AGGCTTTGGGGAAGGAGGCAGGCAAGCTCCCCACGAAGGAGGCTGGCAGCCCTGGCATTGTGTGGCAGGCAGTCTGTCT
CCCCACACAAGGAATTGCTTACTTTGGATGGCCTGGAGAGAACCAATAGAGAATCAGCCTTCAGCCTACTTCTACTGGG
GACATGCTGCTGGGGACTGTTCCCAACAAGGGGAAAACCAGTCAAATGTGGACCAAGCCCTGCTGGGCCTTGTCTTGCT
CCTGGGACTGCCGTGTCCTGGAAACACAGATGGTTTTTGCCACCTGTTAAATGAGCCCCTGCTCCACTCTGGCTCCCAA
GGCTCCTGGGTACAGTGGTTTCTTAGGCCAGATGTCATTCAGTGCTGGCATTAAGCAAGATGTCAGGCTTATAATGATA
CTTCAGCCTGGAGATGAGCATGCCGCTGGCGTGAGGTGCTGCGGAGAGGCTCTCCCGATGTGTGCTTTGGTCCATCCTG
TACTTCCTGTCACCAGAAGGCACTTCTTCCACATACGGATGGCCTTAGAGGAAGGCAGCAAGGAAGGTACACCTCCCAG
GCACTCGGTGCTCAGCACAGGGAGAGAGCCAGAACCAAGGTGGGTGGTAGTGACCTTGCCTAAGCTCCTGCTCCGACCA
TCACTGTGCCGGACCTCCTGAGTCCTGGGCATCTGGCCCGACTCACCCCCGTCACTTGTCTGGGTGGTGTCTGGGGTGC
AGTAGAACCGTGCACACTCGTTCTCCTCAGCTGGCATGACCTTGCTGGAATTTCACAAAGATCAAACGCATGAACACAC
AGCATGCAGGTAAAGCTCGAGTACCAGCTGGCTTTGTCAATAAAATTGTGACGGAGCCTCCCTCTCCCCCTCCTCACCT
TGCAGCTGTTAGGGAAGGAGTAGAGATGCATGAAGCATTTCTGCAGGTAGGCTGCACCTCGCAGCCCCGGGACAGCACA
TCCCAGTTGACAGCTGCAGAGATGGAGGCTCTGAGAGGAGGGGCTTCTGTGTGCCCAGCAGAGCCCAAAGGCAGACCCC
ACTCCGCAGGAGAGAGACTCAGGCCTCTGGCTCAGTGCTTGCTTGCTCCTGCTCATCGCCCGCAGAGGCGGGAGGCAGC
ACTGATATACCAGTTTCCCCACGTGAGGGCTACCCATACCCTCATGAGCACCTCATTCTCACTGGCAGACCACAGGGGC
AGAAGCTGCTCTGGTGTGACCTAGACCGTTCAGATCAGCCTAAGGTAAGCGGGAAGAAGCCCTGGTCCTTATAAATAGG
AAGCATTAGATTCAGAAGCAGAGCTATGGCTGTGGTCTGCTTGAGCAAGTCGTTTTATGGTGAGTGGGAAAGGCCTGTA
GGCATGGGGATGCGGCAGACACAAGGGCCCCCTCTGTGCCAAGGCAGGAGAGGGCTTAGGGCCCCTGAAGAACTGAACA
GGTTCAGGGCTGCTGGGACTCTAGTGCCCTGCAGGTGTGGGAGAGTAGCAGGTATAAGGGTTGAACCTACAGAAGATGG
CAGGGCCAGCCCGAAGAGGCCTCCTTGCCTTCCCACACTTGGTGTCTGAGCCTTTCCTATAGAGAGTGGTGACCCCTGG
AGGCCGGGACAGGGGAGGCCCATGCTCATGGATTAGGAGACAAAGGCAGAGTGTGGCATATTTGAGGGTCCAAGTGACT
GACCTGGGGTCAGACAGGGGGCATTTGGGGAAGTGTAAGCCCAGCTTCTGCCTGGCTCTCTGGCCTACTCAGTTGGGTG
GGTCTGTTCTCTCAGTTTGGTCTGCAGAGACAAGACTCAAGACAATGAGATGAATGAGGGAGGGACTCCCAGCTTTCTC
CATACTGCCCTCAGATGTCCTAGAACCAGCCCCTGGGAGCTCCCCCATCTACACTGCCTCCACAGCAACCTGGGGTCCC
TGGATGCTTTGGCCTTGGTGACATGGGTGTGACAGTCTGCAGCAGTCCTTCCAGACTTCCCTCTGCCCGTTGCTCTGGC
AGATGTGGCATCTGATGCAGTGACGATCGGGCTCTGATTAGCCTCCTGCCGTTCCTCCTCTTTTGGGGGCCCTTGCCAC
ACACGTCCTTTGCCTTCTCCTGTGCTGCTTTCCTTTAAGGTTAGGAGATTTGGTCGCACAGCGCAGATGAAGAGAAAGT
GCTGTTGTCTGCCTTGACGACTGAAACTTAAGCTTAAACTGGCTGCTCCAAAAGCCAAGGTGTTTGAGAGCCAGGGACC
AGAGTGAGTGGAGGCAGAGGACTGCCACTCTTGAACCCACCCCTAGCTGAGAAAGAGCAGCCTATTGGGATCAAATGAG
AACAGGTTTTCAGATGACCCATGACCTCTGCTCTCTTCCCTTCCTGCCTCAGTAGTCTTCCAATGCAACGGAGCACCGC
CTGGTATAGCTTTGGCAGCATGGCTGTAGTTCTGATTGGTTCATTTACAATGACAGCATCTCATAGTGACACTCGCCCC
TTTAGCAGCACCAGATCTATCATCTCTGTGGTCAGGGTGGGTGAGGACTCTTCTCTGTGCTGGTGTTCAGCAGGGGAAA
GAGGGACAACTTTGGCCCCTTCCAGCCTCCCCTGCAGGTGTGGGAGCTGGAGACCCATCCCTGGTTCTGGAAGAGATTG
CTGTAACTGTGACCAGCGCACTAAAGCCAAGTCCCAGGGGACAGTCAGATAAGCTTCTGGCTCACTCTGAGAGTCCCAG
TGAAACATCCCACCTTGACCCTGTGGTGAGCAGGAAGTAGGTCATAGGACAGAGCGGTGGAGCCATTTAGGACACGTTT
CTCATTTTTTTCCCCTCCTGTCCAGTATAGTGAGCAGCCCCGGAGGGAGCCACAGGTGTAGCCAGAGGGAAGAGAGACC
ACGGGTGTGCTGAGTCAGGGAGAGTGTGCTTCCTGCTTCCAGCACAGGCCAGGCTCTGCGGCCACACTAGGTGTGCCTT
CAGGGCTTGCTGGCTTCCTGTGAGGCTTCCAGAGCCAAGATGCTTGGTGGTTCTAACGATGCTATCCATCCTGGGATTC
TGAACAAAGTCCGGTTAAAGACTGGAGGTTCTGTCAAACTGATGCTGATTCCTGGGCTTTGTATGTTGTGACTGTGAAG
GGCAAGAAGAGGAGTGGGGCAGCAATGGAAAGGAGGACTCTGCCTGGTGCTCCCTGGAGCACCCCCACTGCCTGAGCAC
TCCCTGTGTGTGTTGCACCGTGGGGTGATGCTAAGCCTCATCTCTCCCTCCTCCAGGGGTCTGCGCTGCTGCCGCCGTG
ACCCTGCAGATGTACACTTATCTGGCCACTGTAAGAGAATGCTTCCAAATTGTTTTTAAACCCTCGGCATATCCTGTGT
GAAGCCCTAGAACCTGTAAAGTTCTGAATTCTGCCTCACTGGGTTCCCTTCCCCTTCTCTACACCCTACCATAACACTG
CAGGCCACTGCCCAGATCACAAGGAGCAGGAGGCCTGACAAGGCACCTGGCCAGTCTCCCGTCCTGAACCTTTCCTAAC
CACATCAGAAAAATGCCAGAGCCCCACCTGTAGAGCCAGGAAAAGTCTGCTCTTCTCTAGCTCTGAGAACAGAAGAGAT
GCTAGAGCTGGCATCATCTGGTGTCTTCTGGTTGGGGGGGGCGGGTATGGCATGGGGGTCTGGGCAGAGGGTGACCAGT
GCCTGGACCAGTAGACCCTTCAGAGAAATCCTCCATTCTGCCTTCATCCCAGCAGGCTGGGAGAAATATCTGCTGGTTA
GGAGAACTTGTTCCAGGGGATCAGTCCAGATCACAGCAGTAGGAAGGGGAAGGGAACCACTTGGTAAGTGGAAGGCAGC
CCGTTACTGTGAGCCCCATGTTCTCAGATGTCCTTCTCCTGAGTGCAGATAGGACTTGGGACTTGCTGCTAGCCCGTAC
AGTATGGCAAAGGTCGCGGGAGGTCACTCATTTAATTGTGTCATTGGGAAAGGTAACAAGATATTTGTCCCTGGATGGT
TTCATTTTATTTATTAATTAAAATTTACTCTCTAACAATTCCACATGTGTATGCAGTGTACTTTATTCATAACCACTTA
CTTTCCCAACTTCAGCCTCCCCCTCCCCCCCTCAGGACCACGCCAGGTGACATCGTCCCCTGCTCGCCACTATATTTTT
TTGAGGCAGGGTCTCCCAGTGAACCTGGAGCTCACTGATTGGCTAGATTTGCTGTTCGGTGAGCCCTGGGCAATGTTCT
GCCTTTGCCTCCTGTGCTATGATTTATAGGCCACCGATAGCTTTTCACACGGGAGCTGGGGATCCGGTCTCAGATCCTC
ATGCTGGTGTGGCAAGCCCTTTCTCCACTGAGCCATCTCCCCAGCCCTTCTTGGTTGGTCTATTAGAAAACTGTCCCTC
CGTGCTTCGGAGAAATAAGCTGCCATGCTTTGGATAAGTGTGTGTAGAGAGGGGCTGGAAACTGTCTGTGGCTGACCTC
GAACAAGAAAGTGTCTCCAGTTTTATAAGCTTGGAGGAGGACTCTAACTTCGGAAGAGAAATACGGTCTGGCAGACACC
ATGTTTGTGATCCCGTGAGACCCGAAGCAGAAGGTCCTTCAACCCATGTCTGGACTCCTAGCTGGCAGGGACTGAGAGA
TCATAAAAGTTCACTGTTTCCAGCCATTGTAACAATTACCGATGGCAGTCGGCACACAGAAATAGATAGTTTCTACAGA
GGAGAGAAAGATCTTAGGGTGGGGAGATGCCTTTATCAGAGAGGGAGTGGCATTCTCAGGAGGGACCCCTGACCAGCCC
TGAGTCAAGCAGCTACCCAGGCCCAACCTGTGAACCCCTGATGTGTGATCTGCTTATGTTCTTCATGTGTTCCTGTCTT
AGTCGGGGTTTCTATTCCTGCACAAACATCATGACCAAGAAGCAAGCTGGGGAGGAAAGGGTTTATTCAGCTTACACTT
CCACATTGCTGTTCATCACCAAAGGAAGTCAGGACTGGAACTCACGCAGGTCAGGAAGCAGGAGCTGATGCAGAGGCCA
TGGAGGGATGTTACTTACTGGCTTGCTTCCCCTGGCTTGCTCAGCCTGCTCTCTTATAGAACCCAAGACCACCAGCCCA
GGGATGGCACCACCCACAAGGGGCCCTCCCACCCTTGATCACTAATTGAGAAAATGCTTTACAGCTGGATCTCATGGAG
GCATTTCCTCAACTGAAGCTCCTTTCTCTGTGATAACTCCAGCTTGTGTCAAGTTGACACACAAAACCAGCCAGCAGAG
TTCCCAAATAAACCTTGATTCGTGGCATTGTTCCCTGTCAGATGCAGGACTAGAACATTCTGTAGGGCCTCTGTCATGT
CCTTGTGTTCTGACCCCACTCTGATCGATGGCCCTCCCAGGAACCAGGTTTATTGTCCCTTAACCTCTCTGCTGACCCT
TGGCACTTCCCATTGCATGTTTACAGTGGAGCCTTAACAAGTGAAGTCTTCCTGGTCTGGTGACACCTTGGTGACCTTG
TGTTTTTTATTTTTATTTTTTTCTTTTTGTGATTTTTCAAGACAGGGTTTTTCTTGTGTAGCCCTGGCTGTCCTGGAAC
TCACTCTGTAGACCAGGTTGGCCTTGAACTCAGAATCTGCCTGCCTCTGTCTCCCAAGTGCTGGGATTAAAGGTGTGTG
CTACCACTGCCCGGCGACCTTGTGGTTTCATATCAGCAGCAAGGACAAGAGTAAAAAGAAGAGCTTCGGAGGGTGGGTC
TCAGTTGTTCATTGGAAGAGACAGTGCCTGCATGTGGCCCTGGTTAGACAGGTGGCTTCTGAGGGTGATTTCTCCGTTG
TGCATTGGAAGAGATGGTGCCTGGATGTGGCTTTAGTTCCGTACCCCCCATTCCTTCCTCTGGCTTCACAACTCTCTGG
TAAGATCTGCATCAGAGTCTTCTGTGGTTTGGGCCATTATGGGGCTGCTTTTAGGAATGGCTCCTTAGAGACCTTGAGC
TTACAGAGTCAGAAGAAGACTAAACATTTGCTCTGGTTCCCCGTTTCCCCATCCAGTCTAGTGCCGGACAAGCAACCAT
CTGAGTTCCACAAAACTGGAGGAGACACAGCGATCCTTCCTGTATCCCAGATGGCCGTTATCCACTCACAGGCCCAGGG
CCCTGAGGTCTCCAGTGTGTGTTCCTTCCTCATGGTACTTTGGCAGGAGGCCTGCCTCTATGCAGGTGTCTGAGGAGCA
GGAGATGGTCAATTTTGTCTGAATGGGAGGTCTAGAAGTTTCTCTCTTGCTCTTTTTTTTTTTTTTTTTTTAAAGATTT
ATTTATTTATTATATGTAAGTACACTGTAGCTGTCTTCAGACACTCCAGAAGAGGGCGTCAGATCTTGTTACAGGTGGT
TGTGAGCCACCATGTGGTTGCTGGGATTTGAACTCCTGACCTTCGGCAGAGCAGTCGGGTGCTCTTACCCACTGAGCCA
TCTCACCAGCCCTCTCTCTTGCTCTTGTGTTCCCCTTTATAGCTGGCTTGAGCAAGGTAGGCCTGAGCCTCTATCCACC
TCCTCCTGGGCCAGCTGAATGTTGAGCTGCTGTGTGAGGGTCTGTAGGAGGACAGGAGAAGAAGCAAGGGCCCACAGAG
GCAGCATCAGAAGCAGCCCCCTGTCTGGAGACTAAGAGCATCCCAGTTTGCCTCTTGGCACGATGCTCTACCCCTGTAC
CACAGTTCCTTTGAGAAGGGAGTAGACTTCTGGGAAGGTGTTTGCCAGCATCGGTCTTCAGGTCCCTTCCACATCCCTG
AGCAAGCTTTATCCCATTGTCATGTCAAAGTCGTGACCCTAGTTTGAGGAGCTTTAGGTAGTCAAAAGTCTCTCTGACA
ACTTAGGATGCATAGCCATCCATGCACTCTGTCCTCCATCTTGCCACCTCACCCCTTTCTTGTATTCCTCAGACTTTTA
GCATCCACAGCACCAGCCTTGGGAAGAAAAGGGTGAATATGCCCATTACTTTGCCAACATTATGGCTGGATGAGCCAAG
ACACCTATTAGATAAGCTATAAGCTTGCAATAGCAGGAACTTACATTATGGCTGGATGAGCCAAGACACCTGTTAGATA
AGCCATAAACTTGCAATAGCAGGAACTTCCTAAGGGGGCAGTGGGAATATAGCATTGCTATCTCTCTTCTCTGCAGTAT
GTTGTGTTCGTGGTGAACCAGTTCTGCACCGCATGGATGTGTGGACACAGTTGGCCTGGAGGGGCTGGCTGGAGTTGGG
CTGAGCCTCAGAGCAGTTGAATGGGAGCACATTTGCCCATTGCAGTGTGACTTTTCCTGGGAAGCCCAGGGTTGCTATG
GGTACGGTGGATGGGGACCTGCCAGTGTGGTGTGGCTGGCACTCAGATGTTTTTACTTGGCCCCAGAAGGAAGACACCA
TACCACTCTGAAATTGCTATGAGTGTGCAATGCAATGAGGCAGAAAGGATTCAGACCTGGGTAGGTCAGCTGGGAGTTC
TGCAGCCTACAGAGGGTGCCAAGATCTCGGGAGGGTCTTAGAAACCGTGAAGGTGATGTCCTTGATCATCAGTCCCAGT
AGCGAGAAGCCCAGGGAAGTCAAGACGTTTCCACATCGACCCATTGTATTGGCCATATATGTGCATACACTGTCTGTAT
GTGTACTACATACATCTCAAATGTGCATACAGCAGTGGCACCCTGTGATAAGAGCTCCCAGCAGCCACTGACCTGGCTC
AGTCCTGTTCGTCTATGAGGCTTGAATAAATGTACAGTAATATTCCAGTTCCTTAGGAAAATTTCATATTCATCAGGGG
ATATGCAGTCCTTAACAGAGTGAACAGTTCTTTCAGAGGACCCAGGTTTGGTTTCCAGTGCCCACATTGGGTGGCTTAT
GACTACTCCAGGGGTTCTGATACCCTTACTCTGGCTCCGGGGGTTCTGATACCCTTTTCTGTCCTCCTCAGGTACTTCA
CTCACTTTGAGTACATGGCCCCTCACATACACGTGTACTGAAACATGTACTTAAAAATAAAATCTTACAATTTTTATAT
TCAGCAGCACATTTGTTTAATCAGACAGAATGCACCATTTCCTAAATATATCGGGTATCTCAGAGACTGTGTGTATATA
AGCATATATGTTACATATTAATATATAATATTTCCCCTTTGCTGTTCATTTTAGCATACTTTTAAGCTTATAAAAAAGT
TACAGAAATAATACCAAAGCTTCCCGCCTGCCTTTTACCCAGGACTCCCAATTTTTTAATTTTTACAACTCTCACAGAG
TTCTCCAGACTTTTGTGAGATAGATATTTCTCTCTCCATTGAATGTCTGAGGAAGCTGAAACCCAGAGAAGGTGGTAAC
TGACATAAGATCACACAGCAGACCAGAGGCAGAGGCAAGATTAAAACCCAAGCCCGTTGGTCCTTAGAGCATCTGTGTG
AGCAATGAGGCTGTGTGACTCCGTTGACGTCTCCTCTTCTGCCCTGTGTTTCTTTTCTTGTACAGTAGATCTGCGAGCA
GTGAGTTATCTCATCTCATCTGTCTTTATCTCACTTACATTTCTGAAATGGTTTTTTTTCCCCCTGGGGGTAGAATCTG
GGGCTGCCGATATTTTGAAGATGCCCCTTGCTCACTCTGTCATGGCCACTGGAGCCCTAGGCCAGAGGCCAACTCTGAA
GGTTCCAGTGGGCCAGGTCCCTGTCACTTACTCCAAAGAAGGCAGGTGGGATGGCGGAGATGCAGCAAGGAAGGATGTT
AGTCAGCACTGCTGTGCGGGGAAGCAGCCTTTGTTAAGCCTGTGCAGTCTGTCTTCTGGGGACGACTCGGGCTTTAGGT
TTAAAGAGAGGGAGATTGGGAGATACATAAGTAAGCAGGCTTGGTTAAACTGGTCTAGGTGATTATATCAGTTCTGGCT
CTGCCAGCTGACTGAAGTCCTTCAAGGAGGCTTCCAAGGGAAGGGACACTGCAGGCAGGCATTCTGTCCCCAAGGTATC
CAGGTGGTGGCAGGTTTAGAGCCGTGAGTGGCTACTTTCTGGTACGTTGTGTTTCATTCACTTTGTTTGGGGTGGGGGA
GTAGAGTGTCTTGAACAGAACTTTGACAGGAAGATGTTTTTATCTGCATGCTTTCAGGCTTGAAGAGAGCGAGATACGT
CACTGACGTTTGCGCAGACCGCCCCTGAGTAACTAACATGCCTATGTGCAGAGTTAGGCAAAGCCGGAACTTGAGTCGG
AGCAGGACCTCACTGTGTAAGAGACCAGCCTACTTCTGCCTCCAGAGGGCTGTTGGGATTAAAGGCGGACACCAGGGTA
TTCAACTCTGACCAAGTTTAAGACCAAAGAGGAAGATGTGAAAGTGAAGGCTTTTCTCCTGCTTTAGTTTTAGTCTAGT
TTTTGCCTCCTGGGGCCAGTGAGGAGCCAAGTTTCTTGGTAAACGCAGTTTCCACCTGCCTGTTGTATCGCTTCAGAGT
AACAAGGACTCTCATAGTTCTCTCAGGTTTATTGTTATTTTTGTTTAAATAGTTACCTTTAAAGCCACTTAAATGACAA
AAGTCTTGGTATCTGCCCACGATTGTCGATTGTCCTTAACTCTCTTTTGTGTCTGTGTGTTTCTACCTAGTGTCATTTC
CTGTATTCCTGAAGAATTTCCCAGAACAGTTTTTGTCATGAGGATCTGCTAGTGATAAATTCTGTTAGCTTTTGTATGT
TTTTAGATAGATAGGTAGGTAGATAGATAGATAGATAGATAGATAGATAGATAGATAGATAGATAGATAGATAGATAGA
TGGATGGATGGATGATAGACAGAGAGGCCTCTTTATCGTGCCATTGCTTTTAAGGATATTGTCCCTAGGTATAGAATCC
TACGTTAATATTTAGTGTAAGTTTTTACTTTCAGAACTGTTTTTTTTTTAAACCTTAGGACTGGGAATTGAATCAGGGC
CTAGTGAATGTTGAGCACTCCGTTGTCTTCTTGCTTATATCGATTTAGGGAAGAGCTCTGCTGCCCCCTTGTCTTCGCA
TCTTGGTGTCACCGTCCAGCTGGCTTGAGCACCAGAGGCATGATTGGACAGGTCAGAGTGGAAGGCTGGAGCCTCAGAC
TTGGAGCAGTAGCCGGAGTCCCATAGGGGATGTGCCCCACCCCCCAGCCCAGGCCCTGTGTATACTTTCAGAGATGTCT
TAGGGTTTTGCTGCTGTGAACAGACACCGTGACCAAGGCAAGTCTTTAAAAGACAACCTTTAATTGGGGCTGGCTTACA
GGTTCAGAAGTTCAGTCCATTATCATCAAAGCAGGAACATGGCAGTGTCCAGGCAGAGCTGAGAGTTCTACATCTTCAT
CTGAAGGCTGCTAGTGGAAGACTGACTTCCAGACAGCTAGGGTGAGGGTCTTAAAGCCCATGCCCACAGGGATACACCT
ACTCCAACAAGGCCACATTTCCAAATAGAGCCATTCCCTGGGCCGAGCACATACAAACTATCACAAGAGGTTTCTGCTA
ACCACTGGCTCTGTGCAGCTGGGAAATGGAGCTTCTTGGTGGACTCTTCTCCCCCTCCCCTCCCCTCCCCTCCCCTCCC
CTCCCCTCCCCTCCCCTCCCCTCCCCTCCCCTTCTCTCTCTCTCTCCCCCTCCCCACTCCTATCTCACACTTCAGCCTT
TGAGACCTCCAGTCACAGATAGAGAAGACACTGGACACTGCCCTACAGCTAGCTCCCTGGTATTTTGTTCATGTTGATT
CTTTTATAGCTCATCTTTGATAGTGTTTATCACTGTTCGAAAGTTCATTGCTGTTCTCTTCTGTGATGTGCAATCATCT
TTGACATCATAGTTTTCACTGCTGGGATTTTGATTTGTGTCTTTAAAAACCTTTTCCTTGTTTCTGTTTAATTTTTTGG
ACATATGGAATGCAGCAATAACAGTCTCATACGTAATGTTGAAAGACCCTCGTTTCTGGGGAGACTCTTGCTCAAGTCC
TGGGATGAGCTGACGCCCCAATAACTCGAGAGAAACCGTTCTTGATGCAATATCACAGGAGGTTTATTTATCAGCATGC
TGAGGCTGAGCTCGTAACCCACACAGGGGCAGAGGAGATGACCCCCAGCAGTTGCAGTCGAGGGTTTTTAAAGAATAAA
CTGCAAGGTGAGGGGAGGATAGGACCACAAAGAATGGGGAAGCTGAGTAAACACTGAAAAATGGGGATGGTGAATTACG
GCTCATCTCTCACGGAAAGGTTACAGGCTATCTTTGGCAAACATTCTTGGTCCCTTACACTATGGGTCAGGCACCTGGG
TGGGTCACCCAGTGGTCATCATTCTCTCAGGCTGAAGGCGAAAGAACAAAGAACTCCATGCTAGGCTTCGTTTCTAGGG
AGTCTAAGAAACACATTGAGTTGGGGGTCTTACAATGTCTTTATCTGCTAATTCTACTGCATCAGTTTGGGTTTGGTTC
CAGCTGATTTTTGTCTCTCCTTTTTTTCCCCCCAAAAAAAGATTTATTTATTTATTTACTACATGTAAGCACACTGTAG
CTGAGTTCAGACACTCCAGAAGGAGGCTTCAGATCTCAGTATGGATGTTATTGAACCAGCATGTGGTTGCTGGGATTTG
AACTCCAGACCTTCAGAAGAGCAGTCAGTGCTCTTACCCGCTGAGCCATCTCACCAGCCTTTCTTCTCTCCTTTTATGT
TCCCATTTTCAGGCTTCTTTGCATGCCTGGAGTTTTGGGTTTTTTTTTTTTAAGGTTTTAATTTTTTATATGTATGGGT
GTTTTGCCTGCAGGTATGTTTGTGTACCACATGTGTGCCCAGGACCCAGAAGATAGCGTGGAATCCCCTAGAACTACAG
TTAAAGATATTTGTGGGTTCTAGGAATTGAACACAGGTTTCCTGGAAGAGCAGACAGTGCTCTTAGCCTCCGCACAACC
TCTCCTGGTGATTTTCTTTTTTAAATTGTTCAGGTTTATTAATTAGAAGGTGCCATATCCTGACTCTGTCTTCAACAAT
TATATTCCAGGCTTCTTTTCTTTCTTTCTTTCTTTCTTTCTTTCTTTCTTTCTTTCTTTTCTTTCTTTCTTTCTTTCTT
TCTTTTTTTTTTTTCTTTTGGTTTTTTGAGACAGGGTTTCTCTGTATAGCTCTGGCTGTCCTGGAACTCACTTTGTAGA
CTAGGCTGGCCTCGAACTCAGAAATCCACCTGCCTCTGCCTCCCGAGTGCTGGGATTAAAGTCGTGTGCCACCATGCCA
GTCTTATTTTCGAGAGTTATGAACAATCTTTTTTGGCACAGGGAAGGAATTATCTAAGTAATTAAATAAGGGCGGGTAC
TCCTGGCACACTGTGGTTCTAAGCCTGAGAATACAACTGTTACCCTCATGCTGGCTACCCCGGCTCTCCTCTGCCTTCC
CACCACAGTAAGCAAGCAGCCAGACCTTAGTTCTGTCCTTTGGCTTCAGGACTCTGGGCTGATGGGAGGAAGTGGGACG
TCACTAAGATTATGAAGCCCTGGGGTAAGAGGATTCTGCCCACAGCAGTAAAGGAGTCTTTTCTAGAACTTCCATCCCA
CAGTATATTAAAGTCAATTTGGGTCTTAAAGACCATTTGGCTCAGCCTCAATGGAATACCAATGAGCCAAGGCAAAGCC
AAGACGTGTTCAGGGCACATTGGCCAACTTAGGCCTCCATCATTTGACAGGCAGCTCTGGCTGGGTCTTGATGATTGAG
CAAGTCACTGTTATATCAATGAACCCATCTCACTCATGAACAGTGCCTTTCTCAGTTCTGGACTGTTTGTCCCACATGT
CTGTTGTGGGACACAGTGTCTTTATTTCGGTAACCTGAGGCCTTACTCTGCAGAGCCACCTTCAGGCAAGCCCGTCCTG
TGTTTCTAGTTCCTGGTTTCCCTTCTTTAGCCCTTGACCATGTGATGTCCCCTTCCCTTTTGTTGTCCACGTCTTTCAG
TCACTCAGTCCTGTACCTACTACCACCCTGTATGCCTCCTGACCTCAGGTGATAGAGGTTGGGGTCGGGGCTCTATGTC
ATTCCCTGACCTTGAACTGAGAAAGCCTCAGGTCAGAGCTCAAGACCTCTTCCTGATCCTGGCCTGAGGTTGGCTTTGG
CTTGTTTTAGTGTCGTAGGGTTAAGGAATGGGACACTCCTGTAGCTCATGCACCCCAAATCATTCATTCTCACAGTGGG
GCTGGCCAGGAGTAATGCCCGAGGTCTATTTAGGATTAGGGGAGAAGAAGAAATCTCAGTTAGGTCTGGGTGGGCAGGG
ATGATTTGGTGTGAAATCTCTTGAGGCTGTGGGGAAGGAGAGATAGAAGCAAAACTAACGGCTTGCGAGCTCCCAGAGG
GCTTTGGAGGGTAAAGAATGGACTCAGAAATGTGAAGTACTGCATAGTAGTCTGTGTGGGTACTCACCCCACTTGATGC
TGGGTCTCCAGCGCTTGCTTTATAGCCCCCAAGTTTGTACCTTTGACTGGCATCTCCCCATTTCCTCCGACCGTGTGCT
CCAGCAATCACCTGCAATTCTTTGTCCTCTGTAGATTGAACATACAGGTGAGACCATGTGGCTGTGCTGTGTCTTTTTG
TGTGCACAGTATTGTCATTACAAAAAAAGGGTATCAGTGGCCCATGCACACACATAGGCATGCACGCACACACATATAC
CACAGGCACACGTATACACATGCCAGGAAGGAAGCTGTGTTGTGATGAATACATCAGCTGGATTAACTGCCATATCCCT
GCGTACATATCTATAGAGAGAGGGAGTTCTGTCAGAGCATCACCTCCTATATGTTAACTCTTTATAGCATTGACTCGTC
AGCTATGCCTCCGTACACCTGGGGGTGGGGAAAGAATGGCAGCTGGAGAACACACGGATGAGCATCATGCAGGAAGGTG
GCAGAGCCCTCCCTCTGGGCAGCCGGAGGCCCGGGTTGGGCACCTTTGGAAGAGTCTCACCTACCTCAGCCCTTGGGCA
GCTCTTCCCTCCAATTAACTTTCTACTGAAGGAGGAGCCACTGGAAAAGAAGCAAGACAGCCAAGAGTGTATGTGTAAA
ATGGGAAACTGAGGCTGGGCCTCCCAAGGCACTGTCTGGTGGCCGTGTGCAGGCTTAGCAATCCATTGTCATTATGTCA
CAGAGCTTTGAGGAGGGTGTCTGGAAGACTGACCACAGCATACCTCAGGCTCCTAGACAAGGGAAGCAGGCTTGTCTGT
AGTATTAGACTTTGTGGGGAACAAGGAACTTTTATTTTGCCCTTCTGGTTTCTGCCTTATCCTGTCTACATGGCTGAAG
AGTGGGTTTCTTTTTTATTTGACAGCATTTTATTTTTAATTCTTCTATTAATTCATCATCACTATTGAAATTATGTATA
TGTGTCCAAGAAGGCCAGAGGTATCAGATCCCCTGGATCTGGAACTGGAGATAAAGGTAGCTGTGAGCCACCGGACATG
GGTGCCAGGAACCAAGCTCCGGTCCTCTGCAAGAGCTGTAAGTGCTCTTAACCCTAGAGCCATCGCTCCAGCTCCTGAG
AGTTGAATTCTGTGTTGCTAAGCTCTTTCTTTGGATCCCCTGATCGATCCTGCTCTTTCTTGCTCTCACAGAAGGGCTG
TAATCCTCAGGGATTGCAATGGCTTCAAGGTCAACACCCAGTCAGGGCCACATAGGAGGCCAGAACTCCCTTCTGTTGG
CCTGGATCCCCACTTCTCCAGCCAGCACAGAGGATGGTAATGAAGAGACATCTGAGTCAGGACAAGCTTCCAGAACACT
GGGCTTAGAGGAAGTGGGGCTGTGGGGGTGGGGTGGGCGGGGAGACTGAGGCAGGAACACTCAGAAGGGCAGAGGCTCA
GGCATGGCCAGCTCTGGCTGGTATGGAAGGGCCTAAGCCACACTTGCCTGCCCCATGCCTTGCTACCCTCAGCCTCCCT
CTTTGTCCTGCCCCCCATCCCCTTGTTCCATCTTTACTGGCCCCCTAGTGAGGCCCAGGCAGCTGAGCATCCGCTGGTG
GAGGGAATTAACATACTTAATTAAAGTGTCCACGAAGAGCTTTGCATACTTCTTAATTACTGTCTGAGTCTGTGGGGGG
AAGGTTTGGAATGGGATTTTTTTTTTCACCTGTTTCCGAATTTAGCTGACAGTGAGCCAGCTGGGCCCTGAGGGCTCAG
GTGGGAGCTGTCAACACCGCTTTGAGCTGGGGTGGCAGAGCAGGGTGTGGAGGGGAATCCTGGCCCCAGGCACATTTGC
CTTTCCGCAGTCAGAGAGGGCTGGGGTTTCTGTTTCTTTAACCCAGAACAAAAGGAGACAAGACTCAGACAATGGTAGC
TGGTGTCTTCTCAGCTCCTCTCCTCCTCATAGGGCCTGAGTGCACACCACACCGCCACGACTGGGCCAATTTCAGGGTG
CCTGTTGTGCAGAGGTTTCAGGGGGTGGTGGGGGCATCTTTTGGGAGTGCTATGAAACAGTGACACGGGGTCAGGATGG
CCCTGGCCTGTCACTTCCGGTTTCAGGCCCAGGCTGTGAGTGAGCTCTGTCCATCATCAGGAGGACTAGGCTGAATTTT
ATCTCTGTCCAGTGCTGCAGAGCAGTCAGGGACAGTAACGTGAGCTTGAGTTGATGGCAGGCTGCAGGCCCAGGCTCTT
GTAGGTGGCACAGCTTTGGGCAGGCTTTGGCAGATTTTCCATCAAATGATTTCTGCAATGGTTTCTTTTGCCCTCTCAC
TCCCCGGCCCTAACAGCAGTCTCTCCACCCACATTCACACTCACTTGGAGAGGAGAGTGAGGGGGAGGTGTGTCCTTCA
CAAAGTGAGACAGCAGCAGGACGTGGGTCTGGAACTCACCATCTATTCAGCTCTCATCCTGTGAGCCAGGCCCAAGCCA
CACCCAGTGTCCTCATGAGTACTGACTCGGGCATGCCACCCGTGGCACAGTGTGGGTGGATGACGTGAGGTGAAGGGTA
ACCCCATCACCCAGCCCTCAGATCTTCACCACTCCACACCAAACCAGGTGTCAGCTCCCCTCAGAGAGGATGTGTGCTA
GGACCGCCCAGCCCAGTCACAGTGCAGCCCCTCCCTGAAGCTTGGCAGGTCAGGTATTTATGGCGGGGTGTAGCTCCAC
GTGGCCAGGGCTGTTGCTGGAGAGCAGCCTGCTTCCTCACTAAGAACTGGGCTCACAGGATGAACCTGACATAGTTGGC
ACCCAGTAGGGGGTCAGCGAATGCTGAAACTTCACTGTTTTCTGCATCTACTTGGAAACCACCAGAGCGTGCACAAGTC
ACTCTGTCTGGAACTTGTGCTGAGATGGGTCCCAGAGCCTGTGGGAAGCCAGTGGAGTGCATGGACCCCAGGGAGACAG
ACAGGCAGGTACTGGGAGCGGGCCATCAAGGCTCAGCGTTCAGTCTATCTAGCAGCTGGCAGTAGTGCCGTGGTGTGTG
GAAGTGAGCGCCTCACAGCCTGTGGGTATGCGTGAGCCTTGTCCCACAGCTCAGGAGAGAGTTCTGAGAAAATAAGGTT
TTGTGGGAAATATGCATAAATCCTTTGCTCGTGAGATTGAATGATTGCTGTAATATTTACTTCCTGGACACAGTGCTTC
CTTGTGTGTCCTGGTCGGGTCGTTATTATACCTTCATGGTGCTCAGAATCTAAGCTCAGTACCTGATCGAGCCTTGTTG
GCTGGGATTCCAGGGATGGTCACAAAGCCAGGTGACCTGAGACATCCTGTGCTCTCACTTGACTCTGACCTGCCAGGGG
TCCTGTCACCTCCTATGTTAGATCACACCCCACTGCCCCTGCGTCCTTCTGAATAGTCCATCTCTCACCAGACTTCAGC
CCCACCTCCTGGGTGGCTTCTGTAGCCAGTCTCCTGGAGCAGTGTAAGAGTTCCTTTTAAAAGCTCGGAACTGGCTGGG
CGTGGTGGCAGCACTCGGGAGGCAGAGGCAGGCGGATTTCTGAGTTCGAGGCCAGCCTAGTCTACAAAGTGAGTTCCAG
GACAGCCAGGGCTACACAGAGAAACCCTGTCTCTGTGTATCTCATGCTGTGGATCAGACCACCCAAAACTCAGCTGCCC
AGAGATAGGGAGGGTAGTCACCCGAGTCTCTCGAGTTCATAACTGGAGCAGGACACAGCGGGGTAGGTATTAGCTCTGA
ACATACAGCATTGTGAGTGCTAGAGTCCTCGGAATGAGAAGGCCCACGCACTCACAGGTGACTTGGGATGGGAGATGTC
TCGGTTTCTCGTCACAGGAGCACCAGAGTGGTCTCACCCAGGTGGGCTGGCGTGAGAGCCCAGTTCAGGGTGTATAGAC
AGGAGCACACTCCTGGCAGGTGATCTGCCCTTTCTCCCCAGTGACACACAGAACCATTTGTGTCCCCAGAGTAGGACCC
TCCAGACGCAGCCCCTGACGGATGCCGGGCTTGCATTGTGAGGGCATATGGAGTGGGGACCGTCCCTGTGGTGGGAATG
TTGAACCACCATCCCTGGGTACATCCACATCCTGCTGAAGCCTCCTTGGGCGCTCGCGATGCCTTGGATGCTTCAGCTA
TGGCTGCCACACCCGGCAGCGAAGCCTTCTCATCTGCCCCTTGTAGCTCCACATCCGGTGCCAAAGGTGAGGTTTTGTT
TAAATCGATCCTGCAGCTTCATGGAGCTTCCTGTGTCCCTCATGGGAACAGTGTTTTTATTTCTTTCTTCACACACTGT
CTGCCTCCATTATTCTCCCCCTCTCCCTCTCTCCCCCTCTCCCTCTCTCCCCCTCTCCCCCTCTCCCTCTCTCCCTCTC
TCCCCCTCTCCCTCTCTCCCTCTCTCCCCCTCTCCCTCTCTCCCTCTCTCCCTCCGAATTTGCTAGCTCTTCCCCGCTG
CCTTGGGGTCTTGGTCCCACCAGCAGCTTTGCTGACAGACCCCAGGATCTGCTACTTGCCAAGACCTCTCTCCAGGAAG
GAGGCAGAGTGGTACTCACTCCTTCACTGCCTGTTCACTTGGAGACCTTGGGCATGGTTTTTTCTTTTCATTAATTAAT
TAATTAATTCACTTTTCATCCTGATCCCCGCTCCTCTCCCTCCTCTCCTCCCAGTCCCACCCTCAGAAGCCCCCCCACC
CCACCCCCGTGGTTACTAACCCACACATCAGACACATCAGTCGCAGCAGGACTAAGCGCATCGTCTCCCACTGAGGCTC
AGCTATTAAAAACAAAGATATGGGGCTGGCAAGATGGCTCAGTGGTTAAAAGCACTGACTGCTCTTCCAGAGGTTCTGT
GTTCAATTCCCAGCAACCAACCACATGGTGGCTCACAACCATCTGTAATGGGATCCGATGCCCTCTTCCGGAGTGTCTG
AAGACAGCTCCACTGTACTCATATGCATTAAATAAATAAATAAATCTTTAAAAGAAATAAAAGAAAGATATGGTGCATC
CATATGAAGGCAAATAAATGGAGCTAGAAAATACCATCCTGAGTGAGGTTACCCCAGACCCAGAAAGACACTCATGGTC
TGTACTCATAAGTAGATATTAGCCATAAAGTACAGAGTAACCATACTGTAATCCACAGACCTTAAGCTAAGTAACACGG
AGGGCTCAAGGGAGGATGTTTGAATCTCACTCAGAGGGGGAAATAGGCATCGGAGATGACTAGAGGGAGGGAACCTGAC
CTCGGCTATCTTAATGCATAGCATGGAGATGGGGATTCAGGGGTGGGTGGCCTGCTTGTTTCCAGTAAAGCCCAGCCCT
GCTGTGCTGCACTGTGTCCTGAGCCTTGCCGCCAGCAGTGCCAGTGCAGTAGAGTGACCTTGAAGGCAGAGCCCGGTGG
AAGGCTGGCTCTCTGAAGGGTTCCTGCAGGAATTCAAAGCTGAGGTTCACCAGCATTTCTTGCCCCTCCTCTCATTGCC
AATTCTGGTGTGTGCTGACCACCGCCATCCCTCTCAGCACACTGCCCCCCACCCTCGCCATGACCTCTAGTAAGGCCTA
ATGAGGTCTGTTTCCAGATTCCAGCAACTTCTACCCAGGGCCGTTGTCGAGGACAAGAAAGGGACAGGGCAGGACCTTC
TGTCCTCCGGGGTGGGGTGAGGTAGGCCGAGAACTCCCCAAGCATGCCCCACAGGCATGCTGTCCTGTAATGCTGTGGT
GGAGGCCAACGCGAGTGAACAGAGGGACTGCCTTATACTCTGCTCAGGCCTCTGGAGGTTCCAGGAGACAGAATGGAGG
GGGCCTTTTCCTGCAGTTCCTGGTGTCTGCTAGTGTTTTCAGACTTTGTGTCCAGAGCCAGGCAGTCCAGTCCCTTGCA
GACTGTTGAGCCCAGAGACCTCAGAAGCCAGGATCAGGGCAGAGACCCTGCGGTTCCAAACTTGGCCAGCATGGGGCTG
CGGAAACAAATTGATCAGAAATAAAGAGCAGGGATAAATGCTCTGGGAAGCATGAGGACCACGGGAAGGATGAGGCGTG
CAGGGGTGTCTCCAAGAAAAGAAGCTGGAAAATGTTGAGGGGCTCCCCAACACATCCATACTTCTGACCAACTATTTCT
AAATCCAGGGGGTCCCCAGTGCTCCTTAGGTTCAAGAACGGAAACTCAGGAAGCCAGTGGCGGGGGTGAGGGGCAATGG
AGAGATGGCTCAGCTGGTCACTTGCCGTGCAGTCATGAAGACCCGAGTTTGGCTCACCAGCATCCACGTTAGAAGCTGG
GTGTGGTGATGCTGCACATCTGCAACCACAGCCGTGGAGGGGGGAGGGTGGGAGAGACAGGCAGATCCCTGGAGCTAGC
CAGCCTCACCATTCAGTGAGTTCCCACGCAGTATGGCTGTGCCCTCTCCTCACAGAATCACAGCTCTCCCCCTTCAAGG
CATATCTCACAACCCAGGAGGCTATGCAGGGCCTGGCTGTCTGGAATTCTTTGGGATTTCAAAAACAGGCAGAGCTGGT
TGAGTGTTGTCCATGTGATGGAACTCAGTCCCCAGCCTGCCTTCCTTCCCTGGGGGTTGGACTGACTCTGGGCCCCAGC
CCTCTGGTCTTAGGATTGACAGCCCCATCCTGAAGCCACCTAGGGAACCACCACCTCATTAATATAGGCTCAGGTAGAG
TGACTAGGGCTTAGGACAAACAGAGATATTCCTGTTGTGCCCCCCCACCCCCCACCTCCGCCCTCAATTCCAATCGCAG
CATTTTCCGTCCTCACCCCAAGCCGAGGAAGAGTCAGTTTCTTCATTACACAACCTTAGTGGTGGTGGTTTTGTTTTTC
TCCTGTCATTTGTCATTCTGGAAATTACCTCTGCTATCTTAAGTGACCTGACACCTCTTGCTCTTTAGCATTTCCTGTG
TATTGTTCCTCTTATGACCTTCTCACTAATTAACATTCTCTGATGTCTTTGTTGTGTTATAACGAGGGAGGTCCAGAGG
AGTGGTGACCACCTCAAGGTGGTCATGACCACCTCAAGGTGACACAGCTAGTAAGCGCCCCAGTAGGATTTTCACCTCA
GCTGGGCTGGCCATGAGGTCTTCTGGCTGCAGCTCAAACCTAGTCTATCGGACAGGTGACTGCCTCTCCTTGGCCACTG
TTGGCTGTCAGGAATGACTACACTTGTCTGTTACGTGCTGTGCACAATAGATTCAAACCCAGCAGAGAGCTTTTCTATG
GATCCCATTGGATTGACCGGCATGCCACTAGTCCCAGCCTGGGGATGGCCTGCTAGACAGTGGACAGGGCAGAAAGGCC
TTAGGGACCTTTGCCTTCCCTGTCACGGGTACTTATCTCCTGCACTCGCTTCTGAACCTCTCTGGACTGTGGCTGAGTC
CTGGTGGGGCAGAGGAGAGAAGAGCTCCGAGTAGTGACCTAACAAATGTCCCTTTATATTATCCTTCCTCCCCTCCCAG
ATGGCCTGAAGTACAGCAGTTGGCATTCTTGTCACTGCTGTGTCCCAGGGGACCTGCACGGGGTTGTGTCAAAAGCCAA
GCCCTGCAGTTTGGGAAATTAGCAGCATTTATGGCTGTCCCAAGAGACTCTTGGCATCTCAGCCAAACTAGCAAGTCTT
CAGCTGGGGACATTTCTGCCCCAGTAAGGGAGTTTACGGACTGCAGTTGTCACAGTATTTGGGGGGGTCCTGTTGTCAA
TTACGTAGTGGGATCTCGAGGTGCTAATGACCCAGCATTGAGCTGGACAGTCAGACACACTCTGTGGGGAATAGGCTTG
CCCTTGAGAGACCTGAGAAAGGTCTGCTTCCCTGCCCCTTTCTCACCAGGGTTTAAGTTGAATAAATGGGAATCAAAGA
AAGTCTCCCTTGAGCCGATGAGTGGTCAGGTGGTCTATCCAGGGGGGCACAGGCAGCCATGAGAAGAGCATTCTGTTGG
GATCTCATGTCAGCTGGGGCCTGGGGTCAGGGCAGACCTGGACTTGGGTGGTAGAGGGTGAAGAAAGGGTAGGCCGCCA
CCTCCTCCGTGGCAGCCTGGGGACTGACGAGTGTGGCCCCCTAGAGCTCACATCAGCACCTTGGCTTGGGCTCTTTCTC
CTGGGGTCTGCTTCTCTGCTTTCTGTGGTTTCTATGAGCCAGCCTTTCAAGTGCGCGTGTTTAGAAGACCCTAGCCATA
GGCTGGTCTTACGGCTGAACCTCTCTCTCCTTTCTGCCTTGCAGTCGGGAAGACGTCTCTGATCACGAGGTTCATGTAT
GACAGCTTCGACAACACCTACCAGGTAAGACTCCCCACTTCCCATGCTCCCCCCCCCCACCTCCCCTGCCTCTGTCTCT
GCCTGTGGACCCTGTCTGGAGAGCACTGCCAGCTGTCTCCAGGGAGCATTGAGCTCAGAGTGGGTGGGGCCACTGCAGC
TCTCTCAGGCAACTTACCGATTGGAAGGAATGCCATGCCCTTCCTGGTAACCTGGCCTAGGCCGGAAATGAGCTCAGAG
TCTAGATGTTGAAAAGGAAACTTTTATTTTTTAAGGGAGAAATATCAAAACCAGAGAAGGGATCTTTTGAAGAGGCAGG
TTGGAAGCATTACAGAGTTTGGTTATGTCAAGGGGCAGGCCGAAGAGGATAAGATGAAGTGCAGGGGCAGTTGGAGAGG
GTGCAGCTCAGGAGTGGTCACACTGGCTGGACTTGACATACACATAACCCAGACAGACCACGCACGGACTGTCACACAC
GCTCAGTCCGCCCTCATCCCCTGCTGCTGAAGCACACGCTGCCACAGAGAGGCGTGGACGCTTAGTTGACCCTGCCCTG
TGTGGTCAGGAGACTGGGCATAAAGACATCTGTGGAGCCTGGTTTGGGAATGGTAGCCTGGGCTCAGTCACAGGCCCAT
AGGTGACCCCAAACCGAATTCATGAGGGGCAGAGACATATCTGTTTGGTATCAGTGGTGGTGGTGGACAGGGCAGGCAG
TACACGTGAGTTTCCTCACAGCTAAAGCATTTTGCCAAAGTTGTTATCCCTACCACCACCTTGGACTGTCTGTCCACAG
TTAGTTCACCAGTTCATTTCTCTAGGCTAGGGTCCTGTGAGATACTAGCTCCCAGACAATGTTAGTAGCTGTTACTTGG
CAAAGGGAGTGTGTGGTTTCACAAGCAGAGCTGGGTTGTTTAGACTGGGGGGGGGTGGTGGGGGGAATGAGGGTGGGAG
TGGAAGGGGACTGCTCTCGCTCAGCACTGAATCTCAGGGCTCCCCCTCAGCCCAGGGCTTCAGTCTGCTGAAAGCAGAT
GCAGTGGTTGTTTGAATGTAGAATCCCTTTGTCAACACATTCTCAAGGACAAGTATTTATAGATTCACTGGGGCAGCTG
TTCCAAGGTGCCGCTCTCCCCGCTGCTGGGTGTGGAGCAGCCCACGAGTCTCCGATGCTTGTCCGTCCGTCAGTGCCCT
AGTGCAGTCACCCTTGTCTCAGATGTCCTCTGAGGACCCCCTTTCTGTTTCCCTCAAGTTCTTATTCCACTCTGCAATG
GGTGGACTGAAATCCTATATCACGTGGACTCCAGAGGGATTGCCTACGGGGAAGGACAGGAGACTCTGGGTGCGTCAGA
GGGTGTACGTGGCTTTGGGACATTGATGCCGGTCATACTGTTGTGTCCTCCCCCAAGCCAGGACTTTGCGTCACTTTGG
TCTGACTGTGGAGCAGAGGTGACATCCCTAACAGACTCCCCTCCTGAAAGAAAGATGAAATTTCAAGACCTGTAAGTCA
TATAAAGTACTCAGAAATTGCTGGTTGTTTGTGAGCCTAGAGGCTGCCTGGGGCTGAAAATAAAGAAAAACAAACCTGG
GTATGCCACATAGTTAAAACATTCCTGGGAACAGCTTAACCTATAAGATAAAGAAGACTGTGAGGACAAGCAGGGCTAT
CTTGTCTGGGTCAACACCACCTGGCCGGAACCTCCCCTCTGTCTACTGCTCCTTGACGCCGGGTAAAGTCATACATCAA
CTGGTTGCTATGTGAACAAAGATAAGCCCCCAGCCCACAGAAACAAAGTCCTGATGCCCTTTTGCTGACATGTAATCTT
TCTGTTAATGTTTAAATAAGCCAATAGTGTGTCTCTATGCTGAATTCCACACCCCTAAGCCCCTTACCCCATAAAAACC
CCTAGCTTCTGAGCCTCGTGGCCGACATCCGTTATCTCCTGTGTGGGATGCATGTCGGTCCGGAGCTCCATAATTAAAC
GTCCTCATGTATTTACATCAAGATGGTTGTCTGTTCGTGATTCTTGGGTGCACGCCGAATCGGGAGTTGAGTGGGGGTT
TCCCCACTAGGTTCTAACACTCCTAAAGTCTCCTTGTGCTCTGTGTGTGCACATGCACACGTGTGAATGTGTGCACCTG
CGCACATGCTTGTGTTTTTATTAGTTGACTTTTCCCTTTTTGACACAGAGACTCCTGTGGCTCCAGCTGGCCTTGACTC
TTCTGGGTAGCTAAGAATGACAGTGATCTCTGGCTATTTCTGACTACTGCTTGATTTTTTTTTAGCGATCTTCCTGTTT
TTCCTATGGCACCGCATTGAGATTCTTAGCAGCAGTGGACAAATTACCTTTGAAATTTTATCTCCCTTCTTCCCACTGT
CACCCTCATTGCCTGACGCTTGTCACTCAAATCAGGCTTTACTTGTTCCTGTCACTGTGTGGTGGCTGGCGATGGCCTA
GCCTGGACATCAGCTCCTGTCACTGTCCCAGCTCCGCTCGCTCTGCTCTGAAAATTCTTCCAATGCCTGAAGCGTGGCA
AATGTTTGCCCTGAGAACAGAGCCCCACCTCTTGCATCAGTTCCCATGAGCCTTTGGCTGTAGCGTGTGATCCTGAAAG
CTAGTTGGGCCCCTCAAGTCTAGGGCTACTCCTCTGCCCTTCTGGTTCTCACCTGTGTTTTGGGATGTGCCAAGTCCAG
GATCTTCTTGGAGACATCACTGTACAAGGCCTGGCTGAAGATGGTGCCACAGGTACCTACGGGCACAGTAATGGCTGCT
TGCCTCAGTTGACTGGTGCCATCAGACTGAGCGCCTCCCAGGACCACTGTCATGGTGAGTCAACGGAGATCACAGAACA
GATCCGATGCCGCTCTCCCCATGCGTGGGAGAATTGTCTGACTCGGTTTCTTTACATCACCAGGTGGAAATCCTTTCCT
TAAGCCCTTTAGAGCAGTAATTAGGCTGAGAGGCTGGGCTCTGCTTCTCTCCGTCATTCATACACACACACACACACAC
ACACACACACACAAAGACATACACACACACATATACACATACAAAGACATATATATAAACACACAAAGACATATACACA
CAGACACACAAAGACATACACACACATATACACACAGACACATATATACACTCACATATACACACAAAGACATAAATAC
ACAGATACACAAAGACATACACACACATACATATACACACATAGGTACACACAAAGACATACACAGACACACAGACACA
CACAAACTCACACACAAAGACATACACACAGATACATACACAGACACACACATACAAACAAAGACATATGCATAAACAC
AAAGATACACACACACAGACATATATACACAAAGACACACAGATACACAAACACACACACACACACACACACACACACA
GACATACACACACACAGACACATGATTGCTTGTTAATCCTCTGGAGATGGAGGAGGACAGACTTTGTTTATTTTCTTTT
TCTGAGCATAGCAGCAGAGGTGTATGGGAAATAGATAAGTCCCCAGGGACCCGAGAAGATGAAATGTATTTTCTCTCAG
GCTGTCCCACTGCACAGGAGCCGTGCTTGTCACCCTCCCACCAATATCCTGTCTGTCCCAGCAGCAGTACAGCATGGCA
GAGTCTAGGCCCACTCAGTCAGCTCCCAGGACAGTGTTCCCTCTCTCTCCTCATGGCTCCAGACCCTGTGCTCCCCTCC
CATTCACTCATCCTGACAAAAGACAGCTGTGTGCTGTAGTACATGGCCTGACAGAAGGTAGCAGAAGGCAGCCTGGCCC
ACCCTCAGCGGGGCACAGAGGGCAGGGCCTATCCTCAGCCACGGCCAGTGCCACCTTGATGTAGAGCTGAGTGTGCAAG
AGGCCCCAAAGGGCCACCATGCGGCCCTGAGTATGGTAGGCTGGGGTCCTCAGCTGAACCTCTCAGAAGGTTCTGTTTG
TGGCCCTGGTGAGCTGGCACTTCTACAGCCCTGGGGGACCTTTGAGAAGCATGTCTTCGTTCTCTACTTACCTGGCAGT
TACACACATACACACACACACACACACACACACACACACCCCTTATTCTCCGCTAACACATGTACTCTAGTCTCTCACC
GCCCTGCCTAATCACACCATACAAACTGATTCACTTATACAGTTTTCACTTGGTGGAAAACCAAGTCTCAAGCAGAGGA
AATTTTGTCTGTGGTGATTCCTTCATGTAAAACCCCAGCTAAGAGGTAGTATGTTTTGTAATGTGGTAGCATGATGCGG
CGGAAGTGGGTGCCTTAGCGGGCCCATGTTGAGGTACCCCCACCAAGGTACCAGCCAGACAATGGGTCTAGTATAAAAT
GAAGTTTATTTGGGGCATGAGAGGAGGCTGTGGAGAGGGGGTGGAGGCAGAGAAAGAGGACAGAGAAGAACAGAAAGAG
AGGAGAGAAAGAAGGGAAGAGGCCAGCAGAGAACATGCAGGGAGAGAGAAAGGAGGAGAGGACAGAAGCGGGAAGAGAG
GTCACACAGTCTTTTTATAGCCAGCTGGGCTCATACCTGGCTGTTCCTAGGTAACTGTTGGGCAGAGCCTAGAAGAAAT
GCTAACGAGGTTTTCTTAAAACTCTATCTAGGGAAAAAAAAAATCTGGGCATATCTTGTAATAAGAGGTGGTTCTTGCT
GTGCGCTCCCCCACCCATACCAGCACATACCCAGGAAACCCCTGTGGGCCCTGGGAGGGTCAGCTGTGTGAAGGAGCCC
TGCTAAAGGGAGGTGTGGTCCTCTGAAGGTCACTCCTGGCCGAGGCTCTCCAGGGTGCTCTTTCCAGCCTGCAAAGCTG
GCAGCTCAGGCCTTAGCGTCTTAGAGAACTGAGAGGTGGTAAGAACTTAACACTGAGGACCCATGGCCCCTCCTGTCAT
CCACACCAAGTTAGAACAGCACAGATCTAATGGGCTGCCTCAGGGAGAGAGGCGCCCACAGGCCAGGAGGGCTCTTCTC
AGAGGACACTTGTGAGATGGGAAGGTGTTTCAGACTCTGAAGGGGGTTCCTAAGAAGCAAATGCTGGTCTTTCTGGCAG
GCTCAGATCTCCTGAGATCTCTTGCCGGCAGCTGATCCTCGAGCCATGGCAGTCTGTTCTGCCCTGAGGCTTCCCACAC
TCAGTCTTGGGGCCGCCGCCACAGCAAATTACCACACACTGAGTAGCTTCAAAGGAACAGGAGTGTATTCTGTCCTCAC
CTTCCTGGGGGCCTGAGGGTGGAACACATACAAGAGAGCCCTGAGCTTTTGGGGGTTGGGGGGAGGGCATTCTCCTTGC
TGTTGGCTTCTAGTGGCCTCAGATGCTCTTTGTCATAGGGCTTCAAGATTTCCATCTGGCCGCCCTCTTCATGAGATCT
GAGTAGATAAGGTGGATGGATAGGTGTGGATTCTCATCTGGGCCCTTGAAGTGGACGCAGCATGCATCTGTATGCATCT
GTATGCATCTGTATGCATCTGTATGCATCTGCACTCGGGGAGAGGCTCCTAGTCACGTCACCTCACGTGGTGCCTGAAG
CCTTGGATTTTTATTTCTGTCTCCTGCCTTGTCCTGATTACGTGCTCTTGCCGTGGCCACAGAGACTTCTCCCTGACAC
AGTGCGGTAACTGGCCTCTTACCTGACAAGCGATGCTTCCTCTTCAAGGTGAATACATATATGCTTGTACACACATACA
CTCACATGCAGATACATACATGTGCACACACATACACTCTCACACGTGCCTGTGCACACACACGCACACATACATATGC
ATACACATGGCACACACCAGCACACACACATTTATGCAGTGCGCACACACACACACACACACATATACGCCAGCCTTTC
CTGAATACTGAATAAAGCTTCTGCGTGCACTCGTGCCTCTCTCCGCTGTGGCTGCATCAAATCCTCCTCCTCTCCTATC
GGCTCCACTCTTGTCTTCCGGTGGCCTTCCTGCCCACTCCAGCAGCCATCAGTCGCTGGCTTGTCTGGCCCACACACCC
TAGGGACCACGTCACTAACACTGTTTTTCACAGGCTCAGACACTACTGGAGGGGAGGGTGAATGGGCAAAGACTTGGTT
GACCACAGTGATACTTCTCATGATGTGAGCACCATGACCACCCAGGGACCAGGTAGCAAGGTGTTTTCTGTATTAGTAT
TATGATGAACAGAGCCAATTACATGTATGCACGTATGTATATGAATATATGTTATTGTACACGTACACACACGATTACA
AATTGGACAGCCATTCTTCATAGAGAAGAAAGTAACCACGGGACAGATTGTGACCTTGAAAAGCCAGGTGCCTGCCCTG
TGACCTTGGGAGCCAGCATCTCCTCCTCCAGGGCACTAACTGAGCAGCTGTCATTGGCTGGCAGTGACCCGGCCCTACC
TGTTCCCAAGAGGCTGTGAGCTCCTCTCTACAAAGCAACCACTTTCTTGGCTGAGTCTTTAAGTTTACCAGGCTCATTT
GCATGCTAATTTGCATAACTTTACCCAGATACTTCGGTCTGGTGGTTGGCTGTGCCTGAGAGAACAGCAGAGTCATTGT
CAGGGGCTTGTCATGCCCAGCTGCTTTCTGCCATTTACGGATGCTTCTCTCCAGCGCCGGCCGGCCGGGCTCAAAAAGA
AGACGCACGCACATACTTGCCTTTTCATTCTCTTCCCCTGAAGAAATAAATGCTGGCGTCAATTTATGCTAATTGCCAT
GCAGATTTCCTCCGGTAGACTTTATTGCTGTATCTTGTCAGAATGTGGATGCAAATTTATATTTCATAATAAATGCACA
GAGCTAAGAAGATTTCAACAAGTGCCGTAGGGAATATCTTGCTCAGAGATCTTCTTGCTCGGATGTGCTGGTGGAAGGT
CTGTGCAGAGGAATGCAAGTACGCCACCGTCGACACCCCTGTCGATACTGTGTAATGCCACACTCAAAGGAAGATAACC
GTTGGGAGGAAATCTGTGCTTCTTAATATTCTTTGCATGTGTAAAATAAAGTGGCTGCTATTTTTCATAGTCAGTGAGT
GCCGTTTGCCAGCGCAAAAGAATAAATTAGCATTGCCAGGTTCGGGGCCACGTATAACAGTAGACTACACCTCCGTTTC
CTTCAGAGAAGACATCACGCAATACAGGCAGAGAAGGCACAGCTGTGTGAGGGGATCACGGGGCTTCTAGACTGACAGA
GGACTCCCAGGCACATGCCTGTTTGTATCTCTTGATATCTCATCCCCTGTGGGCATCTACCATCGGAAATGATTTGTTT
CATACCTCATAAGATAAAGAATAGAAAAGCTACCTTGACTATAACTCCTTGGCACAGTGAAAGAGCGGCGTGCCTCTCC
TCGGCTCCACCTTCCTGAACCTTTGCTGAAGCCCTGGGAATCCCAGATCCTTGGGATGTCGATGAAGAATAGGACAGCA
AAGGTTCCTGAGGGGCTGGGGGTGGGGGTGGGGGTCTGACAGATTAGGAAAGAAACAGTTCCTGTCTCTCCACTGCAGG
ATTCAACAGAGCCTTTAATGTGCCAGGAGAGTCACTCACTTGCTTGCTGTTTCTGCTCATCCTGAGCAGAGACTCCGAG
GCCATACCGCATAACCAGACAGACACCGGGCAGTCTCCATGGCTTGGCTGTGCCGCTTTGCCTGGAGAGCCCTGGGTCA
GCTGGTTCCTGGTAACAATTGCTCATTTCTGTTGACATAGCATATGACAGAATGAAGTGCCTTCACAGAGTCTTAACTC
CTTTGGTCCAACAGTCCTTAGAGGGAGCTCTGGGATCTTCATTTGAGAGGTGAGGGGGTTGGGGGCTAGGGCTAGGCTG
GGGGCTAATTGGACTCCTTAGTTCAGTATGCACGAGAGCTTGGGTCTTCAGTTATGGGGCACCTGTAAATCCCAGCACA
GGGAGGTAGAAGTGTGAGGATAAGAAGCTCAAAGCCACCCCCAGCAACAAGCTAAAGGCCAACTTGGGAGCTGGGGTAC
AGTAGATCCTTTCACAAGGCAGAAAAACAAGATAAATAAATAAGCGCAAAGAGCAACAAAGAAAATCCCACTGGCGCCC
ACTGGACACATGACCCACTACTGATGGCATGTTGGCCTCTCTGGGGCCAGCTGTAGCCTGTCCTGAGTGGCCCTGATAT
ACACAGCCTGAGTGCGCTCCCTTTCCAAAGAACCCGTGAGGGTTCCTCATCACCTGGGCCGCAGGGCAGCGCTGGGGCT
GTGTGTATCAGAAATCCTGGCATTAATCCTGGAATAACACACACACACACACACACACACACACACACACACACACACA
CTCTCATACTCATACCAGTCTCACGCACCCATTCATGCCTTTGTACAAATACAGTGTAAACTTGAGTGTGTTCCATTCC
CACGCACATATAAACACACATCCACCAATGCATAACTCTCCTGTGTGACCTGTGTTGACCCCTGAGAGGAGGCTGGTCC
AGAGGAGTCACCAAGGATCCCTGGGCCTAGCCGAATAGTTAGTTGTTCTTGCTATCTGGTGTCTCCTTCCTCCAATGTT
CACCTGAAGTGGGAGTGTCCTTCATGACCAAGACTGCAGGTGCAAAGGAAAAAAAAAAACTAGCTCTGCAAGCGAGGGC
ACTGGGCTAAGTGTGGCAGAAATACCGCCTGGGGCTCAACATTTGATCCCAGGTTTCCTGGTGGACAGGAGAAACTATA
TGCTTGGGTCTTGTAAATATAAATACAAGAAGTGTTCAGGTTTGTGTTCTGATCAGATGTTGCTACCCTGTAAACTAAC
CCACATCACCCACAGGCTGGTGTGTTTGGATGCTGGGTCACCAGCTGGTAGGGATGTTTGGGGAAGTTGTGAGATTATA
TTTAGACGACGAGATGTAGTGGAGGAAGTGTGTCTGGGGAAGGCAGAGCTGCAGTGGATTCCGTTTATGGCCTGCCTCC
AGTCCTGATCCTCTGTGGTGGAAACAGCCTCACGCTCCCACCCGCCATGGGCAGGAGCTGCTCTCACCTCCCCTAAAGC
CATCAAAATAGAGCCTTCCTCCTGTCAGTTCTTTCTGTCAGGTATTCTGTCTCAGCAACAGAATAGTCCCCAGTGTCTA
GGGTTTCCTGGGGCCAGCCTACAGAGATGTACACACATGAAGTGGCTTGGCAGGTAGACTGGCTGGTAGTAGTGTGAAC
GGCGAGGTGGACTCCTCACGTGGGCAGTCTGTGCTTCTGACTCACCGGCCTCTAAAATGTGCTGCCCGCCTACCCGCCT
CACTTCCTGCTGGCACCATATTGTACTGTGTAGACAAACACCTGAGACCCGTGGCCTCCACAGTCAGAGCTGACACAGG
TCAGAGCCTGTAGGGTGGTACTGAATAACAATTCAACAGTGCTCTCAGACAGAGCCCAGGAGCTCCACACACTGTGCTC
CGGCCATCCCACCTCCTGCCTTGTGGGAATGCCCATCAGTCTGGATCTAGCGTTGTGGCTCTCCACTTGGGCAGTATGA
GTGATGGTCTGGTACTGCCTGCTTTCACTAGTTGGTGTGGTGTGGTTGCGGCTAACTTTCTGTAGTCTGCTCCAAAAGA
GTCAGCCTATATTAGCTACTTTTCCACGAGTATCACCAAGCACCCAACAGAAACAGCCTAATGTAGAAAAGGTTGATTT
GGGCGTGTGATTTCAGAGATGTCAGTCAGTCATGGTAGAGAACAGAGCAAAGCAGTTTGCATCATGGTGACCAAAAAGC
AGAGAGTCGGAGCATGCAAGCAAGGGTCTGGATAAGGACACACTAGTGACTTACTTCCTCCAACTAGCCCCCATCTCGT
ATGCCTTACTATGTCCCAATAATGCCATTGTCTTAATGAACCCATCAAAGGATTGCGCCATTGATCAGACCAGAGCTCT
CATGATTTAATTGTCTCTGGAAATGCCATTACACACAAGGGCGGGTAGACAGATAGACAGACAGACAGATGGAGAAGTA
CATTATTTATCTCCTAGGTGTTTCTCAGTTTGTTTGTTTGTTTGTTTATCATCCAAAGGCTTATGTTTTGAATGCTTAT
TCCCCAGCCCGTGGCACTATTTTAAGAGACTGTGTGATTTTTAAGAGGTGGGACTCTATCTGGTAGAAGTAAGACACTG
GGGGTGGGAGGGTGGACCCTTGAAGATTATGCCCAACCCCTTCTTCCTATGTTGCTCTCCTTTTCCCGGCCTGCTGTGA
TGTGAACAGCTTTGGCCACATTCTGCTACTGCCAGATAGCCTTCCCAAGCAGGTGGGACCAAACATCCATGGACTGAAC
TCTTTGAACTGTAAGCCAGAAGAAAGTCATCCCATCCTTGAGTTCTTATTTCCATTGAGTATTTTGGCTGCAGTGATTC
AAGAGTCTATACTAATCTACTTAATAAACTTAACCACCATACCTGGGCAGTGGTGGTGCACGCCTTTAATCCCAGCACT
TGGGAGACAGAGGCAGGCAGATTTCTATGTTCGAGGCCACAGAGTGAGTTCCAGGACAGCCAAGGCTATACAGAGAAAC
CCTGTCTCAAAATGAAAACCAACCCCCCCCCAAAAAAAAAAAAAACAAAAAAAAGCCAAACTTAGCCATCACTCAGCGC
ACAAAGGCCATCTTTATGTCCTCATCTGAAGCCATGTCCTAGTAGCAGAGAAGGTGTGGGGTGGTCCTTGTCACCCTGC
TGACTCACTATCACACCCTGGGAAAGTCATGTCTCTAGTCTGAACCTCTAGTTTGTCATCTGCTGAGTGAATATATGTT
CACCCATATACCGGCTACTCCACTTGTTAAAACCTGCCTCAGCCACGCACACAGCCTTTCTACATCTGTCCACATTCGC
TAAGTCTGACCTGTCCTGAGTATCATCCACATACCCTGCCCCGGAGCCTGCTCCTTCCCCTTGCATATCTTCCAGATAT
ACACAACAGTTCTGTGTATCTTTCTTACATCTGTCCCATCTACCAGCCAGGATGATCTTTCTAGAGTGCCGGCTATTAG
GAAGTCCTTCTGAAGTTTCACTTCATCCAGTTCCCTGGCTTCCTCAAGTCCCCTACAGCAGCCATTGGCCCCAACTGTT
TTCCATTTTCTGAAAGAACTACCTGGCTTCATTCTTCAGGGTGCTCTCTCCTACAACACTCTCTTTACTGCCTTTACCT
GTGTACTGACCCTCTCCTGTGCCCGTAGTCCTTGCAGAAGACCCAGGTCCTGTCACCACCATTTCCTTTCAGGCTAAGG
ATCGTCTTTAACATAGACTCTTTGTCCCCGTTCATTTAAAAGGTGCTTTTAGTTTATCTGCACATGAACATTTTTTGCT
GGGTTTTAAATTCTAGCTCCACTTGCTATCTGTCATCATTATCTTCCCTGCTCTTCCATCCTCGGGTACCAGGTGCCAT
TTTCCTCTGGCTGGTCTCAAGATTGTATCTTTGGTTTGGTCCTCATTGATTTGACTCTGATTTTTAAAAGCTTTTCTGG
GCTGGAGAGATGGCTCAGTGGTTAAGAGCACTGACTGCTCTCCCAAAGGTCCTGAGTTCAAATCCCAGCAACCACATGG
TGGCTCACAACCATCCATAATGAGATCCAACTCCCTCTTCTGGAGTGTCTGAAGACAGCTATAGTGTACTTACATATAA
TAAATAAATAAAATATTTTTTTAAAAAAAGCTTTTCTGTACTTCTTGTCAATTGTGGTGGCTTCATGTTCTGTCCTTCA
CGTTGTGTTCTAGTTGGTATGATATTATATACTTGTGTATGCCTATGTGTGTACTGTACATGTTCTTGTGTGTGTGAAT
GTGTGTGTGTGTGTATGTGTGTGAAGAGGTACATTTCTCATGGCACGTGTGTGGACATGAGAAGACAACTTGTGGAATT
AGTTTTCCCATTTCTACCATGAGGGTTCCTGGGCCAAACTGAGGTGGTCAGGCTTGGCAGCAAGCACCTTTACCTGCTA
AGCCGTCTTGCTGAACTTGCCCATCTCTGTCTTTGTTATGAACATAACCTTCTGTAGTTGGGCATAATAAATATTGGTT
ATCTTAAAATATTTCTTCCTACTTTCAATGTCCGAATCATCTCAGGATTGATCCTCATTTTCCTTGTGTATGGCTTGCG
TTTTCCTGTCACCTCATGTGTCTACTCGGTTGAGATTACATCCTGCGCGCGCGCGCGTGTGTGTGTGTGTGTGTGTGTG
TGTGTGTGTGTGTGTGTGTGTGTGTCTGTCTGGATTCTGGTTGCAGCTTTGAGTCCTGCTGGTTCTTTGTTTTAAACCC
TGATTCTCAGCTCTGTCCCCTGTGCTGGGCAACAACCCTCTCTTCCATCTGTAGTCTTGTTTTGCTTCTGGCCTTGGGC
AGATTGCTTGGGATCTTCTCTGGCCATCCAGTATTGGACAGCATTTCTATAGAGACTTCAGAGCCCCTTCTGTGGCTTT
CTCATGTCTAGGATTTTTCTGTACTTCTCGTCAATTGTGGTGGCTTCACGTTCTGTCCTCTCGTGTTCCAGTTGGTATG
ACTGACTGGGTTTCTTCATGAGTCTGAGTTTTTACCAGCACTTCCTGGTGCCTTCCTCGCCTGTTTTTTCTAGTTGATT
CAGAGTTAATAGCATCAATCATCCAGAGGGCTAGATGACCTCGGATGATGAGAACCAGAGTCCACCTCACTCCTCCAAG
ATGCTTCTCCTATCCCCACTATTCCCCGACTTCTGCCTACTGGAAGGTCCTATGTACTGAGGTCTTGCGTATCCACTCC
ACATATCCATCAGACACCGAGCAAGCCAGGCCTGCTGCTCTGACCTTCATCCCTTCCACGCTGGCTCTTCCTCTCCATT
TGCTGTGAGTGACAGTTTCTGTATGTGTCTTCTCCTCCAAGTAGGAAGATCCTTAGGGGCAGGGACTGGGGATTTTTTT
TTTTACAACACACAGTAGGTACTCAAGGGTGAAGTGAAGTGGTTTGACTTAATTATTTACACCATCTGCCACGAAGAAG
CTGCCCAGGGAAGACCATGACTGCCACCTTCTCTGAAAATCTGGCCTCCGTGGGAGACACAGGCTCCAGCTGGCAGCGG
TGTGGGCCAGCTGCTGAGACTCATGTCTGGGAAATACATGGTGCCCAGGAGCTAGAGCTATGTTCTGGTCATGGAGACC
CAGCCAAGTCTCTCTTTCAAGCCCTTTTCCCTATGTCCTTTGAGGGCTCACTTTATTTATTTCTACTAAAATGCAAATG
AACTCTCTCTTAAGAGGGCTCTGCAGAGCTGTTATGGCCAATGATTACCGCTTTCTGACAGTCGGGAGCCAGACAGGAT
TTGACTCCAGGGAAACACAGTGACAGCTTGGAAAATGGACTTCTAGAAGCCGCAGGACTACAGCTCAACAGCCCAAAGC
AGAGCCTTTGACGTCCGGGAGAGCTGGATTCAGATCTCAGGTCTGCTACCCAGCCTGGGTGACAGACCTTGGTTCGGAG
TGAGCCTGGTTCCTTCCCTGTGCATAGCAACTCATCCACTGGTGTTATCTCCATCTCCCCCACCCTCTTGTTATCAGTC
TGTGGCCCTGGAGAGCACAGCCTCCTCAGAGCTGGATCACAGCACAGAGCAGTTTATCTGCACGCTTCGCTGAGCAAGT
CACTGCTCTGAGAACTTGTCTGTGATTCTGACACGCAGAGCCTGTGCTTGTGTCTACCTGAGTGGGCCCAGGAGTCGCA
GTGTTTAAGGAAGCTCCTGGGGTGACACTGAAGCACATGATCCACATGGTACATGCTAAAACCCCCCACATGGTTGACC
AGAGGCAGAGATTAGGGAGTCTGAGACATTTCCTATTTGGTAGAGAAGGTATTGAATAGCCTCTGGGCTATCCCAAGTG
TTGATAATCTGATTTGCGGAGAAGCATCTCAAATTCATAAGTACTGATTATGGAAGAACATTTCACAGGAGGGACAGCA
TCAGGAAAGAGATGCAGAAGCTGACATGGGAAGGGCCTGTAGACTGACTCAGTGGGTAAAGGTGCTTGCTGCCAAGCCC
GGTGACCTGAATTTGGTCTCCAGATACATGTGGTGGAGGACCAGCTCCAGCAAACTGTCCCCTGACTTCCACACAAAAG
CCATGCATGCATGCATGCACACACACATACAAAGTGAACAAAAACATGCAGAGAAACATGTGTCTTAGTTAGGGCTTTA
TTGCTGTGAACAGACACCATGATCAAGGACAACATTTCATTGGGGCTGGCTCACAGGTTCAGAGGTTCAGTCCATTATC
ATCAAGGCAGGAGCATGGCAGCATCGTGGTGCAGGAGGAGCTGAGAGTTCATCTTCATCTGAAGGCTGCTAGAAGACTG
ACTTCCAGACAGCTAGGACAATGGTATTAAAGACCACGCCCACAGTGACATACTTTTTCCAACAAGGCCACACCTCCTA
ATAGTGCCACTCTCTGGCCCAAGCCTGTACATGACATGTGAATTCTAAACAGCATTATTCCTTGGTATAAAGTCAAGCT
GGTTATGAGGAGTACAAGGTTATAAAAAGCCAGTGGTGACTCTTGAACAGTCAACCACAGGTGCTTAGACTGTTTCCTG
GGAGCTGTAAGGAGGAACAAAGTCAGGATGTGTGTGTGTGTGTGTGTGTGTGTGTGTGTGTGTGTGTGTAATATTTATA
TTGTATATAAATATATAATACATATTATATATATTATTAGTTTTGTTTCTTAAGATTCATTTATCTTTATTTCATGGTT
GAGTGGTTTGCCCCCATGTAAGAAAATGCACCATATATGTGAAGTGCTGGTGGAGACCAGAAGAGGCCATTAGATTCCC
CAGAACTGCTGCAGACCCTCTGGGCCCTTTGTCTGCGTGGAACGGGTCTCTCTAGTCGCGGGTAGGCAAAGCATCGAAT
GAATTGACAGACAGATGCACACAGGAGAGGTTGTGTAGAATCTGAATGTAATGTCAAATCCAGCATCAGACTTTTTATA
CAGAAGACAATAAGGAAGTTGGGTGACATTCCTGCACGGTAAAAAGAGGTTACTGGATTCTTACATAAAACAGAGGAAT
GCAAACACAGAAGGTCTAACAGGAACCACCTGGGATAGAATTCATTGTTAACAACTGGGATCAACAAGGGCTCCACCTA
AGATTCACTTAATCTTAGAAGCCCAGGGTCAAGGGCTTCGTGCCCTTGCCATAGTTCCTATTTTAGTCTATTGTATAGT
CCACCTTCCCCTTAGGCCATTGTAAATACGTGTGTGTGTGTGTGTGTGTGTGTGTGTGTGTGTGTGTGTGTGTGTGTGT
GTGTGTGTGTGTGTGTGTGTGTGTGTGTGTGTATGGGTGTAACTCAGCTATAGTCTTAGAAGCCAGGTGTGAGGTCTTT
ACTCTTCTAGGGCAAGGGCTTTCACACCCAAGTCATGGTTCTAACTAGGGAGTTCCTTTCTAGCTAACCATCTCATGAA
TAATGCAATACTCTAAAACCATAGCCCGATCTACTTCCTAAACCATTGTAAATTCCTGTATATGGGTGCGACTTGGCTT
TTATTCTAAGTGATAGTGTAATACCAGAGGCTATTTCTGGATGTCACTGAATAGCAATATTCTTACTGAACTTCTAGCC
CATGGTCTTGCTCAAGGACGTTCTAAGACTGTTGGAACACTGGCGGAAGGCTTTAGCTATGTCAGAATTCAATCTTAAA
AAGCACTTATAATAGGATAATACTAAAAGAGAGCACGTGGATCCGTACACTAGACTAACTCGGGAATGGAAAATTAATG
TATGGGTTAGAGAGAACGCCAGACTCCAGGAGTGTTTCCGTGAAACTCTTCCGCCTCATGAGTAGCTTCCAAACCTCTC
GGCTTGTCAAACTGACTCAACCGGAGTGCGTGGCATAGAACAAGCTCTATGGAGAGTTGTAAGCTGCCCTGTGGGTGCT
AGGAACTTAACCTGGGTCCTTCCCAAGAGCAGCCAGTGTAATTAACTGCAGAGCCACCTCTCCATATCCCTAATGTCAT
TGGTTTTAAACTCAAAACTAACCTTGCCTGCCACATGAAGGTTGCTTATGGGGCAGTGGTGAGGACAGGGAACCCAGGG
AAGATGCTACTGAGGTTACTCCCGTGGGAGGTTCAGTCTGCACGAGGGAGCAGTGAGATCGGTGAGAAGGGGTGAGGAT
GGGAGCAGGTGAAACCACAAGAGGTGGCACCGTCATCTCTGCCTTCCCTCACCCCTGTCTTGATGGTTTCAGAATGTCC
TCAGAGTTCTTGAGACTCATTCCCACATCTATTTTCTTCTTTTAAAGCCTAGCTATTTGTGTCATCTAGGGGTCTGTTC
CTCTTGAGGTTTTTTTTTTTTTCCTCAATTATGATTCAAATTTCTTCTTTGCATAGGTGGTCTGTTTTTATTTTGTGGA
CAGGATAATTTAAATGCATAGCTTTCTTTGCTTCCCCAGAGACTTTAATCCCTTCATTGTTCACCAGCTTGTGGTCTCT
CTTACGTGCTTGTACATTGTTGTTTCTTGAATTTTTTTTCTAAAAATCAGAATAGCCTGATCCAATCTGCTCAGCTCTC
AACAAAAGAAAAACAGTGACATCAGAGCTTAAGAGCAAAGATGGCTCAGACACCCAGAGGTGATGCCCATGCTGTGCTG
TACAGATGTAAGAAGATACACATTAGTTGAATGAATTACATTTTTTATGTTGTTGTGATTAAGTTACCTGACAGAAACA
GTTTATGGAAAGAAAGAGTTTTGGTTTTTTTTTGGGGGGGGGGGTTGTTTTTTTTGTTTTTGTTTTGTTTTGTTTTTTG
TTTTTTTTTTTGGCTCATGGTTTCAGAAGAGTTCAGTCCTCATGGCAGGGAAGACACAGCAGGGTTCACAGCAGTGCCA
GCTTGTGGTGGGAGCTCCTCAGACCACAGTAGACCCTGAAGCAGAGAGAGCAAGAACCAGCTATAACCTGGGGTCCGTG
TCAGACACCCACCCTAAATGTCCTGTCTCCACCAGCTAGGTCCTGCTTCCCAAAGGTTCCAAACTCTCTCCAAAGAACA
CCACCAGCTGCAGAACAAACACTCAAAACCTAAACCTGTGAGAGACTTCCAGTCAAGCCATAACATTCCATCTTGGTTG
CCAAAGGCTCACGGCCATCTTAAAAGACAAAGACACACTTATTCCAATTTTAAGACGTTCTCACAGTCTCAACATTGAC
CAATAGTCTAAGTCCAAAGTCTCCACTGATGGTCAAAACAGTCTGTGGTCTCCTGGAAATAAGAATGTGTTCAAGATTA
GAAAAAAAATAGGAGACCAAAAGCCAACAGGGGAAACACTTAAGTCTTCTACCTCCGTGTCCAGAATCTGGGCACATCA
TTTGGTCTCACACAGAGATGTTCCTTAAAAATCTAGGTGCTAGTCTCTGTGACCCCATAATTTACATTCTGCACACCTG
AAAACTAGCACCAGATAGGTAAGTCAAAAGTCAGCTGCCGCAGGGACCCCTTCGATTATGGCTGCTTTTGGCTTCTGAG
TACCTGAGAAGCTGAACCAAAACCTAGAAAACCATCTTCTTAGGTGGCCCCTTCCCATCGGAGCACCTTGGTGACTCTT
CAAAATACATTCACCTTCTGAAGGAAAGTATTTTAAGTGATTAATTGATTCGTCTTCTATTTCATACCCTGGAGCCCTC
AGAATGGGGACAGATTCTCAGCTCACCTGCTCTGTTGCCCCCGTGCCAGGTGAGCATCAGTAGCATGCCGTGACCCCTA
GTCTCTTATCCACAGCTTTAAACATCTTCTCCTTTCTTTCAGGGCCAAATTACACATCTTGAAACTTCTCTGCATTTTG
CCCCCAGTCTCACTGTGAAGGTAGCTAAAAGTAACCAGCCAGAACCATACTGGTAGCAGGTTGACCTCTGTCTGGGTTG
CTTTGCATTTTCTCCATTACCTTTGAAGTAGTCCACCACCTCACTCAAAATTTCAGGTCATGGGTAGAATGTAAATAAA
TTCTTTGTCAGATTCCAGTGGCCTCTAGAAGCCAGTGACCAGTGGAGTCCTTGTTTTGCCCAGAGATCACGTGGGTATG
GTCTTTGCTGCTCATATTGTTGTTAGTGTTTGATCCTCCAAGTTCTCAAGGGAACAGTGGATCTGGGGCCTTAGAGTCC
ACATGGTTAAACTGTTGCAAGCCAGTTTCAAAGGCTTAGAAACCACCTGGTAAGATTAGCCATCTGAATGTTGCCACTT
TGCTGGGATCCGCTCTCTGCTTCTCACTTTAGCATGGTTCTGAGAGAAACAACTTGAAGAAAACACTCTTTCTGGCTCG
TGGTTTTTCAAAGGGCTTCAGTCTATCCAGCAGTAGCAGCATGAAGTAGAGGCACCTCACGCCACTGAGGCAGATCAGG
AAAGAGCGAGGACAGAGGCTTTGTGTAACCTTCAAAGCCTGCCCCCACCCAACTGCTTCTACAAGGTATGTCCCATTCT
GAAAGGTTCCACCGCTCTCCAAGTTAACACTGTCAGCTGGAGAACAAGCATTCAAAACACGAACCTATGGGATGTTTCA
GATTGGAACGGCCAGAATGAGTCTGAGAGAGTGACAGCTATGCGATTAGGGATGGCTCTTAGGTAGTAGCTTGAAGATC
AGATAGAGGTGGGAGGCAGCTGTGGGGAGAGGAAGTTGGTCCCTCTCGGCGTTGGCTCATTGCTGTCTCCTTGGTCAGT
CTGTTTCGTGAGGCTTAATAGAGATATAGTCTGTGGCTCAGTGAGCTTTAACGATGATAAAAGTCACTGGGCTACGTGT
TGCTGCGGCTTGATCCAGTGGCATGACTCCTCGTCATATTATCTAATGATCTCTCTCTGCGCCATGGCAGCGAAGTGTC
TTACATGTGCACACAATGGGGGAAAAGGATTGTCGCCGTTACAGTAACATCCCTGTCGCCACCCAGCTGCCGACCTTAG
GGGATAGGATTTTCCATGGTCCTTCACTCTGTTGATCCAGGTTAGTTACGCCGTACTGACGGCGGCTTGAACAAAGTAG
AAAATGAGATCTCTGTTACTTGTAGTACTGCCGAGGTGACTCTCTTCACAGTCCAGGAGTCAGGCTCCGTTGAAGTCTT
CCTGCCCTCTGGTCCTCAGCTGGACGAGCCACGATCACCCGCCCCACGAGTCTGCATTCCACGCAGTGGGAAAGGGGGC
AGCTTAGTGGGGCGCACTAACCCAGGAAGGGCTCATTCCGGATGATCATCACCTACACATCCTGCGATCAGAACTCAGC
CACCCTGCAGCCAAGGCTTCTGGGAAACATGGCCTTTGACTGGGGCAGCTCAGTTGTGCATTTTCTAAATTCTGTATTG
AGGGATGCTGGCAGCCTCTGCCTCTGATCCTGAGCCCAGGCATCTGGTCTTGCTTCAGGGGTCACTAAAGAAGGAGTCT
GGTGTCTTCAAGGATCCTGTGGCACATAGAAAAGTTGACATTTCTGCCTGCTTGGCATATTCCTGGTCTTCGAGCGCGC
ATCAGTTTTAGACTTAGTTCTCATGGCTGTGGTAGGCACTTGGGGAAGTCAGGCAGGAAGCTGGAAGCAGGAGTTGAAG
CAGAGGCCATGGAGGAACACTGCTTACAGGCTTGCTCCTCAAGGCTTGCTCAGCCTGCTTGCTTGTAGCATCTAGAACC
ACATGCTCAGGGCCTTCCTGCATCAGCCATGTATCAGGAAAATGCCTCCCACAGACGTGTCCACAGGCTAGTCTAGTCA
GGTGGAGCCAGCTGAGAGCCCCTCTTTCTGCAAACCTTTCTGTATTACAAGAAGATGATTCTAGGCACAGGGTGGAGCA
CGCGACTGGAGCCATGCCCTGGATGTCTAGCTGTGTCTGAGGGTTCGTTATCACTTAGTGGTGTTTGCTCTGTTTCACA
CCGACAGTATCTTGGCTCCTGCTTGTAGACATGGGGTGCAGGTAAGGACCAGAGAGGTTGTCTACTAGGACAGAAGAGG
TGCCAGGTGGCACAGGAGTGTGGGTGACATCCCTTGGCTTGGCAGCATGAGGAGCAAAAGAGCTCTTTGGGCTTCAGGC
ATTGTGGATGGTCCACAGTACCCCATCCAAGGCAGGGCACGATGCTCTCTGTCCCCATGCAGTGACAAATCCTACCCAA
CCCTGCCTCCCCGAGAACAATGCCGAACTGAAGTCCAAGTCCAAGGGAGAGTCTAGGAGCTCAGGGGATGCTGGAGGCA
GGTGGGGACTGCTTCCTGTGCCCCATGCTCTCTCACCCTTCTAGAGGCACCGTTCTCTCTCTCTGCGTAGAACATGCTC
TGCAAACAAAAACCTGATCTCACCATCATTTGCTCAATCTCCTTCCTCAGTGGAGGGACTTGGCTTGAGTGTCCCCTCT
GTTGTCTGTGCCATTTCCTGGGGTCCCTGGCCTTCTGGCTCCTTTGTGTGATTCCTTTTTCATGTTGACCTTTTGGTGT
TGGTTTCTTTTTTTTAAACAGGCAACCATCGGGATTGACTTCTTGTCAAAAACCATGTACTTGGAAGACCGTACGGTGA
GTTCCTGGTCTTAGAACTGGAGGACAAGGAGGGCATTGTCGGGCTTGTTCCCTAGAGCCCTCACTTGAGCACTCTTGGG
AGTCTCTGCCAAGACCAGGAGGGAGCTAGCAGTGCCATCATCGTGGGGTCTGGGCTTCCAACCCCCTCTACCACTCCTC
TACCGATGCCTCTGTTTGGGCCTTGTAGGTTCGACTGCAGCTCTGGGACACTGCTGGGCAAGAGAGGTTCCGCAGCCTG
ATCCCCAGCTACATCCGAGACTCCACGGTGGCTGTGGTGGTATATGACATTACTAGTAAGTTGGAGTCTGTGTTGGAAG
GAGGGGGCTCTTAGGACCCAGTGTGACCCTGACCAGACCTGTGTGTGTGTGAATGTGTACGCCAGCGTGTGTTCAGTGT
GGGGTAGTGTGTTATCTTAAACTGTAGTGGATTCATCAGTGTCCTTCAAACTCCTGTGACAGATTCGACTGCATAATAC
AGGGTTGATCCTGCTTGGGGCACGGGCCCCCCGGGGTGGGGGAAATGGCAGGTGTTCACAGGGTTAGCTCTCTGTGTCC
TCCCTGGCCCTGGCTGGCATGTGTGTGCCAGCTTTCCCCAGGCATCTCTCGAGGCAGTCCCTCTTGTATACACGGTTTT
TGAAAGGGCTGCACAGACTCAGTGGGGGACCAGACATTGGGGCAGGCCTTATGCAGAGCCCAGGTACCCCCAAATCATC
CAGAGCAAAGATTCTCAACCTGTGGGCTGACACTCTTCTAGGATCAAATGACTCTTTCACGGGGATGGCATACCAGATA
TTCTCCGTATCAGATACTTACATTGCAGTTCATAACAGTAGCAGAGTTACAGTTTATGAATTTGTTACTGTAGCAACAA
AAATAGATTTATGGTTGGGGCTCACCACAACATGGGCAACTGTGTTAAAGAGTCATTGCATTGGGAAAGATGAGGACCA
CTGAGCTAGAGATGAGCAACTGTGTTAAAGAGTCATAGCACTGGGAAGATTGAGAACCACTGACCTAGAGGAACACACC
TGGGAAGCCCCAGGCTTTACTCCCAGAGTGCTCTGAGGACCCTGACATCACATGCGGTTGTGCACGCCTCACTGCCCAG
TTCTGCCTTGCCCCTCTGTCTACCTCCTCCTCTCTGAGCATATGCTCCTTACACCCCCTAGTGAGGTGGGAGTTGTGTC
ACAGGCTGGGGAGAGCAGCCACCTCCCCCCCACCCCCAGGTGCTGAATTTGGATGTCTGAGGGAGATGACCCTGAACAC
AGGTGACTTGCTTCTGTGTTGATATGCCTCTAGGGGGCGCTTTATCTGCCTACCCTTCCCTGCAGGGTTGCAGGTTCTC
TTCCCCAGCTAGCCTGGCCTTCCTCAGGCTCCTCTGAACATCACTTTGGGGGGACACCATTTCCTAGGGGCCGCCATAT
GCTCCCTTCACAGGTCCGCGGCTCGGCCAGGCCATGGCCAGCTGTGACTCTTGCGTGGTCTCTAGAGTCACTGTTCTGT
GTAGAAATTGTGACAGCCTAGCTTCCTAGGTACGGTGACTTCTCTCCCTAGGGTGGCTGAGCAGTGCTGGAGGTTCTCC
CAGTATCACAGGGTAGGAGGCCATCTTTCCTTCTGACCCCTCCGGGGACCTCATTCCCACTCTGTTCCCACTTTCTGCC
TCCAGATCTCAACTCCTTCCAGCAGACTTCTAAATGGATTGATGACGTCAGGACGGAGAGGGGCAGTGACGTGATCATC
ATGCTTGTGGGTAACAAGACGGATCTGGCTGATAAGAGGTAGGCAAGCACTGGAGTGTGGAGGTGCTATGACTAACGGG
CCCATGCTTCCTCGCCCCTCTGTGGCTGTTCCTGCCCAGGTCTGAGATGACAGGGCAGCGAGGCTGATCTCCACGCACA
GGGCCCCCGACACGCCTGTTAGCCCCCATACACTGCAGGCTTCTCATCTCTGATTTTCCTCAGCTCAGCAAAGGCAGGG
CAGGGCCTGTCTGCACAGGCATTTCTATCATGGTCCCAAAGTAGCCTGTGACACAGCTTCTGTCCCTGGGAAATCCTGG
CGTTGACATGTGAGGTCCTCAGGTATGAGCCTGAATTATGTCAGCCGAGTACCGACTTTGTCCCCTGCCCATGCATCCT
GGTTTATGGCTGGATCTGGGCATGGAGTATTTTTGGAAAAGGTTTCTCCGTAGCTGTCCCCTGATGTCACTGCCCATTA
AGGTGTCATTAGCATATATGTCCGCTCTTGGAACCCTGTGCTAACTGGGACAGAAAAGCCGTGTGTCGCGGTTTCTAAT
AAAGCATGTGGCTTCTTGCAGCAGCCCTGCCGAGGGGGGAGGGAAATGGTCTCCTCTGCAGAGCAGTATGGGCCAAGGC
GAGCTACTTTTAATTTTGGTGTTTGCATTAGGAAATGGGGGCTGAGGCTTCACTCCAAGAAAGGCTGCATGTGTCCAGG
AGATCCTGTTCTAACGACTGCTGAGAAAGCCGTAGGGGGCGTGGCTCAGTCGCAGAGCGCTCGCCTTTCCAAGTGTGAA
GCTCAGCATCATAAAGCCAGGCATGGTGGCATATCCTACAGTCCCTGCTCTAGGGAGGGACATATCATACTGAATCAGT
AGTTCAGTGTCATTCGTTGTTACATCACAAATCCAAGGCCAGCCTGGGCTACATAGGTAAAAGGTAGAAGGTAGCCTTG
CTTCCTAGTTCAGAAGATGTGGGAAAATGTGGAAAAGACTTGGGGTTCTCTGTGCCCAGGTGCCACATTCTTGCTAGAC
AGACTATAGCAGGCCTCCTGGCAGGGACCCCTGGGCAGGAAACCTGTCTTCACGGCTGACCAATGGGCCTGGCAGTCCC
TACAGTTCTTTATCGCAGGCTGCCTCCTCTACTTCTGCCAGGGGTTGCCTCTCTGCCCCCTGATTTTCCTTCTCCCCTT
CATTTCCAGGCAGATAACCATCGAGGAAGGAGAACAGCGGGCCAAAGAACTGAGCGTCATGTTCATCGAGACCAGCGCG
AAGACTGGCTACAACGTGAAGCAGGTTAGGATACAGCTGGCTTGCTCGGGACTGGAGGCCCTCCCCTGAGGTCTTCCTT
GCTCTGAGTGGCTAGCGACTAGGATGGGGCAGACAGCCAGTGCATATACTTATAGCTCTGGGCAGTGGGGGATGGCACT
GGCTTCCAGCGCTGGTAAAGCTGGTGGCGTGGGGGGTGGGGGAGGCACTCCGCTAGGGAGCCGCGCAGACCCTGTCAGT
CACTAGTCTGTCTCCTCAGCCATCATCAGGTGTGGGTCCCTGGAGCAGCGTGTGTGAATGAAAGTCCAGCATGGACTTC
CACCTGGCTGAGGAAGCTGTGGTTCTTAGCATCTCATTTCACTTCACATGCAAATGTAGGATGTTGAATCTGATCGACA
CGTTGGGCTTATGTCTTGTACTTGGCAAGAACCCGGGGCCATGAAGGGTAACTCAGAAATGGAGCAAAGAGACTCCCTT
GGCAGTCGGGAATCATGGGAGGGAGCCAAGGTGTGGCTGGGACCCACAGGACCAGAGTAGTTGGTGGGCGGAGCTTAGC
AGCTCAGCCGACCTGGGCTTCAGAAAAATCCCACAGGTATTGTGACTGCTCCCTCATCTGTAACCAGCTCTTCAGTGAT
GAATACTGACAGCCAGACAGCCTCAGTGTTAGCAGTGAGTCCTGTGACTGTCCCACTGGCAGAACTTCACAGTATGACA
GGGATCCCACACTTCACAGTACTGGATCTCAGCATCCCACTAGCCGTTAGACCACCTGTAATCCTGATGTGAACATGTA
TGTTTGTAAGGAGACACGGATGTCAGCGTGGCTCAGATTTATGATCCTTAAATATTTGGGAAGTTTTATTCAGAAGCAA
ACGGTTTCCTTTATCATCCATTATAACTTGACTACATCTGAAAAAAACGAGTCCACCGGCTTCAGCAGTGTGAAAGGAA
CCCATGGCTCAAACAAGGTGAAGAACCCCTGTCTCGGAGCAGGAGGAGGGTGGTAGCAAGGAGGAAGGACATCCTAGGC
AGGGCTGATGGGCGTAGAGCCTGGCAGCAAGGTGCCCGTAGCACGGGGAGGGACCCTGAGTAAGGAACGTGGCTTGATG
CCGCCGTGTCTGCTGCGGCCCTCGACTGACTTCCCAGGCATACCTGGGCACCACTTGGCTTTTGAGAGAGAGTGATGGT
TCACTTCATATTGATAGTGCTGAGATTAAGGTTGACAAAGGAGCTAGAAAGGACAAAGTAGGAGGGGCACTGTGGCCAT
ATTAGGGAACCCCCGGCCCCAGCTTCCGGAACCCAGTGCCCAATTGGAGGGCCCTTCAGCTCCATTCACTCCAGCCCTT
TCCAAGCCTCTGTATCAGATGTGAGCCTAACCACCTCAGAGAGGAGAAAAAGAGGCGGGGGTGGGGTGGGTGGTGAGGC
CGCTGCTCTTTGGGTCCAATGACATCATGGGAAATCGAAGCCGAGGACAGTATCAGGCATCCATAGAGCAGGAGGCATC
TTCAACCTGCTGGGCCAAGGTGAGACTGGTCCCTTCCCCCATCCTGGAATGATCTGGAATCTGAGCAAGGGTTTGAGCA
AGAGTTAAGTCTGGTGGGTTTGAGCTGCTATTGCAATTGCTACAGGCTATGCAGCTCACAGGTAACGGAACCTTCTTCC
TCCCTGCTCTGAAGGCTGAGGACTCCAAGATGAATGCGCTGGGAGATCCAGTGCCTTGCGGACTCTGCTCCCTCATATT
TGGCACCTTCCAGTTGTTTCCTCACATGAAAGGAACGTGAAGGCTCCCTCAGCTTCTCTTCCAGTCAGGAGGGCTCCTT
AAGGCAACACCTCCTCACACCGCCTTGCTAGGAGTTAGATTTTTTTTTTTTCATTTGTTTAGTTGGTTTTGTTGTTCGT
TTGGTTGGTTTGCTTTTCAGTTTTTCGAGACAGGGTTTCTCTGCATAGCCCTGGCTGTCCTGGAACTTACTCTGTAGAC
CAGGCTGGCCACAAGCTCACAGAGTGCTGGTGTTAGAGACATACACCACCACTGCCTGGCCAGGAGTTAGGTTTTAACT
TATGAACTTGGGGGAGACCCAGACATCCAGCCAGGCTTAGAATCTTCTGTGCTCTTGGCTTCTGCTTGTACTTGTTACT
TTCAAGATGTCACCTCTGCCACTGTCCATCTATTAGGTGCCCCTGATACCTTCTGTCCTCGTGCACACTCAATTAGGTA
GACTTGCACGTCGACCCACACTCCACATGCTTGCTTCTCCCACACCCACTTTCTCATATACACGCAGAAACATGGCATT
TACCATGATACACACACATACATACACACTTACACCCAAAAACACACTTAACTTTTTATACCCACTCTACAAACAATAT
CAGGCATACCACGCATAGCACACGCATGTAGCACACCACATTGACACAAATTTTCACATCCTTCACTGTGGCTATGGCC
CTGGCTCCCAGGATCTGTGTCCCTGGGGAGAGTCGTTTGTCTTACGTAAGGCTAGAAACTGGTTTTCTACACCTAGTCC
CCATGTGAGAGTGATGGACAGACACCCACTGAAGGGCCTGCTTCCTGTGGCCTGGATGATTTCTGTAGCAGAGCAGGTC
CCTTGCAGTCACCCTCATGACCCCTGCTGACTGCCTGTGGGCCAGCCTGCCTCTAGGAAGAGCCCCAGCTTTGCTGTAT
GTGTGGACATGTGTTTGTGTGCAGGTGTGTGTGGGCATGCATGTGTTTACTTACACCTGTGGCGGTCACAGGTTAACTT
CTGGTGTCATTCCTCAGGTGTCATCCACCTTATACTTTGAGATAGGGTCTTCGACTTTACCTGAAGTGTACCAGTTCAG
CTAGCCAGGGAGCCCTGGGAACCTGTCTTTGTCACTCCATCACTAGAATTATAAGTGTGTGCGGCTACATGCAGCTTTT
TACGTGGATTCTGGGGACTGGATTTGAGTCCTCTTGCTTGTGTGGCAAGCACTTTGTCTCCCTAGCCCCTGTTCCCTCT
TCCTATTAACATCCTGAGTCGCTCCAGGACCAACATTGAGAGGGGCTATGGTGATGGCAGAGGACTTAGAACAGGCCTG
CACTCTGGGGCAGTCAGTGGGCAGTCAGTGGACAGACAGGCCACCAATCCTCAGGCTCCGTACACGAGGAGAGCAGTAC
AGGGGGAGACCAGGAGAGGGAAGGAGGGGGACTGGGCAGGCCTCACCCTGCATCTCCTGCCTGCAGCTCTTCCGACGTG
TGGCGTCAGCTCTGCCCGGCATGGAGAATGTCCAGGAGAAGAGCAAAGAAGGGAGTATCCTTTTCCTTTATCATGTGAG
TGAAAAGATGGCTCCCCAAGCCCGCGAGTCGTTTAGTTCAGTAAGCAGATGAAGACCACCTTGCCTCTGTCGCAGCCCT
CCCTGGTCTGATGCAGAGAGCCGGGCCACCTCTTTCAGTAGCCAGATGGGCTCCACGTTCTCTGAGCTCCAAACCTTCT
CTAACCAAGGCTGCCTCTTCTTTCTTAGCAGTGAGGAGAGACAAGGGCTGTGCAGCAGTGGGGGATTGGAGGGTGGTGG
CTCTGTGTTGCTTTGTAGTGAACACTGCGTGTGTTTCCTGAGTCCCCTGGAGATCCCAGACAAGGAAGCTTCCATGCAA
TGAGCACTTGATTTCAACCTGCCAATGCTTCCTCCATCTGCCCACTGCTCTGTGGGGAAAGCTTGCCCAACACTCATCC
AGTATAACTAAAAACCTCGCTTACACCCAGATGATGAATCTGAGGTGCGTAGAGCAATCCAAGGCCCGCTGTGTGTGTG
TAAGTCAGGGAATTAGCCTTTACACTGAGACTTCCTGCATATCTACCCCTACTCCGTCCTGGAGGGTTGTCTCTCAAGG
ATCCCAGCGAGGCAGAGATTAGTAGCGCTGTAGGTGGCTGAGGACATTGCCCACAAGCAGGCTTTCTTTGAAGTTTATC
AGATGGCCCTACCTGAGCAAAAACCCACCTGCAGAGTCAAGAAAGGAGCAGGACCGATGGGAAGCCAAGAGGTGTAGTG
ACCCTGTGCCCACTGGGAGGGACAGATAGCCTGCTTTTATCCTGTCCACTCCAAAGGCAAAGCTGACATACTAGCATGC
ATGGCGGTCATTCATTCATGTTCTCCCCGAGGCCCCGGTGGCACTCCATGCAGGATTCACAGTTGGGTCCCCCAGGCTC
CCACTTCAGCATGACAACTAACGTGGCGTCCTAGTGAACCTAGCGACCCGCCCAGCTTTAGGGAGAAGAGCGCTGGGCA
GATGGCAAGCAGATCACACAGGGCACTGTTTGCCCAGCTGTGGGGTGCCCCCATCTGGCAGATGCAGCCATCCGTACCA
CTCCCAGGTGCTCATGGCTGGAGCCTCCTATGAGGCAAGACTGGAGCAGCCTGGAGCAGACTCTATAGACAGCCCAGCA
GGGGACAGGTGGAAGCAGCTGCCACCTCAGCTTGGTGCTGAGCCAGCACTTGTGTTGACTGATGGTTCAAACCCAGATC
CTGTCATGATGTGTTAGGGATCATGCTTGGGATTGCATCAACATGAGGAACAAGCACACAGAAGTGCCACTTGGTGATA
TATGAACGGTTCTCATGTGGCCTTACTCCTTGGACTGGTGTTGGGGGGGGGGGTGCAGAGCTTCTGTCTTCTGCTTCCA
GGTCCCTAGAGGGTGTCTGCACTTCACATCTGCTCTGCACAGGAAGCCTGCCCAGGGCCTGTCTAGCAGGTCCCCTTAC
CCACCCTCCAGCGCTTGATCTCTCAACAAAGTTGAGCTAAGGGACCACAGAAAGATAGGGCCACAGCCCTTTGCTGTAT
CCACACACACAGGGCACTCCTTTCTGGAACAAGGGTTTCGTGTGCCAAATAATGACAGTAACTTGGCAGTAACTAATAG
ATTTCTCCACCAACGAAATGAACCAATTCAAGCCAGATCAAAAGTGTAAAGGCAGGTTTATTGAGAGGCAGCTGTCCGA
CGAGGCACACTTGGAGGTCAGTAAAGTCACACGTCCAGGCTAAAGCAAGGGAGTTATGTGGGTTTTAGGCACAGAGTGA
TGACGTGTGAGCTAGGAGCTGGGATTATGCCTGTGTAAAGTAGAGAAGCTGTGTGGGCACTCACTGTGCGTGGCTGGAA
ACAGAGACAGGGAGAGGGGTTGTGTTAGCCTGGAATTTTCCTCTTGCAGGGCCGGGTTGGGGGAAGAACAAATGGGGTT
TCTTAGCTTGTGCAAGGGATATGCAGGGGCTCCAACCAACCTTCCTGACCTCTTTGGGTAGAAGGAAGGGCACTGATTC
AGAGCTACATCCTGCTGCAGAGGGAGAGAGTCGGGAGTCAGTGGGTCCGAGTTCCTCGGCAGGTATGAGTGCAGAAGGA
TTTGGTTGACAGTTACTCGGGAAACTTTGAGAATCCGTTCTGGGAGAAAGCGGAGGATGCAAGGTGCTAGGAGGAAAAA
AATAGATTAATACCACGGCCCTGTGAACGGGGCTAGCTAGGTGCAGAGTGAAGACTGCCAGGAAGAATAAAACGGAGAC
TACCCTGGTTGTCTTGGCAGGGTTCTTCAGAGTTTGTGAGGTGACTCAATGTTAGCCTCTACTAGGCCAGGTTTGCTGG
CATAACAACATTCTTTTCCCAGGGACATACAGGTGCCTCCTGAGGGATGAGCCTGCTGTGAGGAGGACCAATGCTCCTC
ACAGGTTTTGCATGGCCACGTGAATTGATCTCATGCTGGAGGAGTGGCCACCAAGTTGTTGATGGGCTTTTGGGGACAG
TCAGACACCTCTCTGATCTCAGCCACCTGTATCCCATGCCCCCCGCAGTGGAGGTCACAGCAGCAATACCTGTTACTAC
TGTGATCTGTGTGGGCAGAGGGCACCTGAGATTGGAAAGAGCTGGTAGGATAGGAGAGCGTGGCTGTGTCAGCCTTTCC
ATAAAGCATCTGGTTCGGTACTGGTGTCATTGGGATACACAGAGAGGAAGGATCACCAGGAGGCAAGCTTTTTGCTATA
GAGCCGCTGCACCGAAGAATCCCCTGCTGTGCCTGGGGAAAAAAACAAAGTTGTGTTAGTAGTATTGCAAAGGGAGCCA
GGTCCAGAGTAGCAGAAATGGAAGTGAGTGTCTGGAAGAGGAGTGCGGCTTGTATAGTGATAGCCTTTGGTTGGAGGGT
CAGCCCTTGGCCTCACGAAGGGACCAGCAAGGGAACTGCTCTCAGAAGAGCGCAGCCCAGAGCAGCCCAGAGAGAGCAG
TTGTTGTTGTCAAAGACTTTAGAGAGGTTAAGTAGGATTGCTCCCTCCTGGATGAGGCCCAGCCAGGGGAAAGGTTCAT
TTTCTTTAGTTTTTACCTGGGGTTGGGAGGAGTCTCTCGGGTGTGATTGGAGGGACTCCTGTTGTGGTGTGGTCTTGGT
GAGGTTTGCCGGGAGCTGGAGGGAAGAGGGTCTGGATGTCGGGTCTCCAGATTTCTCTCTGTTCACATGGTTACGAGGA
TGTTTCGCTCTGATAGACTTGGCTTATTACCCCGAGCCCAGCGGTCATTCCAAGGATCTCAGACGCTAATATAGCTGTA
GTTTAGTGGGGAATATCCAGCCACTGGGCAGGCTCATCCCTGTCCATGCTCTCAGCAGCTTTCTTGTCTGCACCCTCCT
CGGATGTTCATTTTACAAGGGCCCCATGAGCAACCCCCTTGTTCTTTGACCCTTTGGGAATGACAAACTTCCTCCATCT
GGTGGTCGTGGGTAAAGCATTCATAGAGGAGCAATACTCCCTGTGCATTTAAAGAAAGATCTGGGATGGAAATACCATC
TTGGCACCCCTTAGAAGGACAGTCTGTTTGGCCCGCGATGTCGTTGTGGGTTTCACCGGCTTGATCGCAGAACATGGAA
GCAACAGCAGTATGTTGGAGCTGATTTGGTCTCACCTAGCTGGACAACCAGGCAGGGGTGTCTCGGTGTCTGGCACCTG
TGGCAGCAGAGAAAGGGTGTGACCCAGGAAAAGGAAGAGGCAAATAGAGTGAATGTTGTCACGTTTGACCATTCAGACC
TGGTTTTGAGAATCCCTGTAGAACCTGAGAGCGGTGGGTGCCAACTGCTAAACAGACCAGCCATCTTGGGCTGGGGCCT
TTTTGAGCCGAGATAGATGGTGCCAACACTGATATCCTAGGAGTTTGGCAGCTGAGGGAGTGGTGATTACCATGTAGAG
TCCGATCCACTGGAGCTCTAAAGATTTGGAAGACAGTTGTTTGAGAAGCACCTGGTCCCCTGACCCCGGGAAAGGGACA
GGGGTGGGCAGTGGCTGAGTCTGCTGATATTGGGGTAGGGAGATAGTTGTCAGTGTGTGAGCCGATAAGGGAGCACGGG
AGGGATAGGGTAAGTAGCTGGGGGAGTCCGGGCCCGCGGATGTTGGTTGAGGAGAAACAGCCTTCCGTGCATGAGCTCA
GAAGGACTGAGGCGCGTAGGAGAATGTGGGGTAGCTCAGAGGTGAGAACACTTGGGAGAAGGGAAGGCCAGGGTAACCT
GGGCTCAGGTGAGAGTTCTGAGTTGTTTGATAAGGCTCTCTCTACCTTTCTCAATGACTGGGTGGCACGGAATGTGAGA
TTTTCGGGTTCAAGGCATCCCTGGCATGCAAAACTGAGCTCCTGTGTCCCAGGAGCTGTCACATCCGCTGCTCCCCTGG
AGGTGGGGAGCATTTGGATCCACCCTGTAAAAGTGTCAACGAGGGTTAGGAGACGGCAGAGTCTTTTCTGGGCAGACAT
GTGTGTGAAGTCAGTTTGCCAGTCTTGTCCTGGTTGGTGTCCTTGGAGCTGGTGCAGGGTCTGGCGTATCTGGAGAGCC
CCCTGTGAGTTTGTCTTGGCACATGTTTGACCGGAAGGATGAGGCTGTAAAATATGGGCTCAAAGGTCAGTGGAGTCAG
TGGGGAGGGGCAGCTTTAGAAATGGGAACAGAGCCTTGGGTCTGATGTGTAGGCTCTGGTGGATGTCTGAGATAATAGA
AGGTGCCAATTTGAGGGGGAGAATAAAGCTGTTATTTAGGCAAAGCCATCCATCTTTTGTCTGTGTGGCCCCTTTGTTA
AGGAATCTGGCTTTATCCTCCATCTGACACTGAGGTTTGTAGGAAGATCGTAGGAAGAGGATGTGCCCCGTGCGTCTGT
AGGAGCTAGGAGCCAGACCTCAGACCACTGAGTCAGCCCTGGAATTATCCACAGGCTCTGGATCTGTGGAGGTCTGGTC
CCTCCCTTACTCCTGCGCTGACTTATGGCCACCTCTGCAGGGAGTTGGAGGGCTCCAAAAGCTTGGCTATAAGGGGACC
ACCCATGGTGTCAAGGAACCCCCTTTCCTTCCATAGGACAGAGGGGCTGTGAGCTACAAGAAAGGCTTACTTTGAGTCA
GCATTGGCTCATTGTCCTTTGTCAACATGGAGTGCCCTGGTTAGAGCAATGAGTTCAGCCTTTTGTGAAGAGAGGTTCC
ACTGCGAGATGGTTCGACTCAGTCATTTCAGCTGAGGTGACCACCGCGTATGCCGTCTGGTGGGTCCTGAGTTATCTCT
AAGGGAACTTCCATCCACAAAAAGGGTGAGTTGTGGGTCTGTGAGAGGCTGGTCTGGGAGGCTGGGTTGGGGTGAGCGT
AGGTCCTCCACAGCTCTAAGCAAGAGTGGGAAGGCACATGGAGGAGGGTGGACCAGGAGGAACAGCTGCTGGGTGTAGA
GCAGGGCTAGGGGCCAAGGTGACCTGGGAGTTTCCCAGGAATAACAGATAAAAGGGTTGGGCCCATGAGGGTCTGAGGA
GAGAGAAAGAAGGGTGGCTCAGGAGATCAGAGAGGCAGCGGGTAGAGTATACAGTAATAGGCTGGAGTAAGGTGATCTT
GAGTGAGTTCAGCCATTGCTGTTAGGTCTCGCAGACATGGCTGCCATCGCACACAGTAGGGTCCAGTTGTTTAGATAGC
AGAGCAGTGGCTGGGGGGACTGGAGAGATGGCTCAGCGGTTAAGAGCACTGACTGCTCTTCCGAAGGTCCTGAGTTCAA
ATCCCAGCAACCACATGGTGGCTCACAACTATCTGTGATGGGATTTGATGCCCTCTTCTGGTGTGTCCGAAGAGAGCTA
CAGTGTACTCATATGTATTAAATAAATAAATCTTTAAAGAAGAAGAGGAGGAGGAGGAAGAGCAATCAATGGCTCAGTG
AGAGGGCCCAGTGGGTTGGACTAGGGCCCCAGCCCCTCGTCAGTAAAGAGGTGATAGGACTGAGTAGAGTCTGAAAAGG
CCAAGACTGAGGCTGCTAAAAAGGCATCTCAGAGAGTAGGGAAAATGATGGTGAGCTATAGCTGAGTGGGTGAGGGGCC
CACTGGGGGCCCCCTTGACTGCCTGGTATAGTGGCTTAGCCATATTAGCTAAATATGGGAATCCAGGGTCTAAAGAATC
CAACCAAGTCCAAAAATGAAAGAATATGATCTGCCATAGTTTGGGGCTATAGATCTCTCAGAACCCGCGCTTGTTGAGG
GTCTTAGAGCCTCGGCTAAGTTGAACACCAAGTATATATTACAGTGGGAGAGCATAGTTGAGCTTTAGATGGGGAGACT
TGATCCCGGGTAGACAAGGTAGTTTAGAAACAAGAAGTTGGCCGTCTGGGACAAAGACAGGGAGGGGCTACAAAGTAAT
AGGTCATCTACATACTGAAGACGTGTATTTAGCATTAAGGTCTAAGGAGAGAGCCAGGCAGTGGTGGCACACACCTTTA
ATCCCAGCACCTGGTAGGCACTGGAGGGCTTACGGAGAGGGAGAATGGAAGAGTTGCAAGTCCTATAGGGATCAGAAGT
CCCTGACACGCGAGGTGAGTATTGATGGACTTGAGACAGTGACGATGGGGCTCAGAAGTGGGGAGCTGGGGTCTGAAAG
GGGAAAGAAGAGGGGTAAAACTAGGACCGGCTGGTGGGGCGGAGCGATTACCAGGGTGGAGATATACCATACTTGGGGG
TCAACAGGGTCTGGTAGAGTACATCCAGGGTAGTCTGGCGTAGGGATCTGTGAGGCAACCAAAGGAAGAAGAAGATGAG
AGTGGGGAGAGGTTGTGGGGATTCAGAGAAGTGGCTGAGGATATTGTGGTCAAGTAAAGGCACAGGGCAGACAGGCATG
ACTAGGAAAGAATGTGTAAAAAATGGTCAGCCAAGGATATAGGAAAGTGGCATAGTCGGCATGAAAGGAGGGGATCCCA
TCCATCCTCATAGCAGAAACTGATGAAGGAACAGCGGGATCCGAGTGGGAGGTCAAGACAGAGAAGGTAGCCCCATGTC
CACCCGGAAGGTCATGGCACATTACCCTAGGATCTGCAAGAGTGACAGGAGCCCTCGAGTCTGGGTGCCATCAATCCTG
TGCCAGGCCGAGGAGCCCTAAGGCAGGAAGAGTTTGTGCTACCTGCACTGATGGCTCTGGACCTTCGTGACCTGGCCCA
GAGGACAAGCCCACACTGGGGCATTCTGACTTCCAGTGGTAGGGCAGTGAGCAGACAGCATGGGTTGCTGAGGGCTGGG
GCAGTTGTGGGATCAGTGGCTTTGCTGGCCGCATTTGAAACAGGCCCTTGTTGGAGTTGACATTGACGGGATGGGGTCT
GCGGGATTGCCAGTGGCCTCAGGTCAGTTGCCAAGTCTTAGGCTTGGAGTGCAACCTTTTGCTGTAGCTGAGCCTGTCG
GGAAGACTCAGTTGCCTCCCCTTGGGCATCGAATACCTTAAAAGCCATCTTTACCAATTCCTGTACGGGGGTTTGGGAA
CTATCTTCAGTCTCTCCCCGCCCCCTCCGAATATCTGAAGATGACTGGGAAATAAAATGGGTAGCCAGGACTGTAGCCC
CTGCCGGGGAGGCCTGGTCAAGGCAGATGTGTAGACCATGGTTTGTTGTAACCAGTTTTTTAATGGCCAGGATTTCATT
AGCTTCCTGTGTAATTTTGCAGAGATTGCCAAAAGTCAACAATCTTATTTGAGACCATTTCCATTCCCTGAAGGAGACA
ATGGACCGTACTCTAATCGGTGGTCAATGTGACCTGGCTAATAATTCCACTTAGGCTCTGTGGTCAGCACCGTTGTGTC
ACTCACAGGGACTGTATTGTCCACTAGGTGGCCCTGGTCTGCATACTGTCTGCCTGCAGCCTGAACCTGTCCATGTTCC
TCAGGAGTGAGGGTAGAGCCTGAATGACATAAAGATCGTGGAGTTAGGTCATAGTCCTGACAAAGGAAGCAGAACTCTT
TAATATAGGCAGATGGGATGGCAGAGAAGGGGCCTCTCCACTTTTCAGTCTGGGAGAAGTCTTGAAGGGAGAAAGGAAC
ATGAACTCCCACAGCTGTTTCTGCTCCGGCCACCTCCTGGGAGGGCAGAAGAGCTTTGGAGTATTGAGACTGCATAGGA
GAGGCTACAGGGGAGGGTTCAGGGGAAGTGAGCCAGGGAGTGCATGGGGATCAGGGTGTGGGTTGACCCCTAGGTGGAG
CCTGTGTTGGTAATGAAGGAGGAGGGACAATGGGAGGAGAGAGGAGGGGAGGGACAAGGGGGACAAGAGTTAGGGGGAG
GAAGTTAATTGTCCTTGGCTTTTGTCTCAGAGGGAGGGGAAGAAAAAGGGGTGGGTACCAGGGAGAAGGCTAGCCAATA
GGCGGGTTATCAATGGTGTAGGAAGGAGGAAGGAAGGAGGAAGGAGCAGGTATAGGGAGGAAGGTTGGCCAATAGACTC
AAGCTTATGGTGCTATAGGAGTGGGAGTAGTAGACAGGGCTGGGGAAGGGGTTGGTGTGCCCATGATTCAAGTGGTCCC
GCCAGGGTAAAGGGTGTCTTGTAGTCCAAGTCCCCAGATGAATGTGTGTCTGGAGGTGTCACAGCCTTGGCCAACAAGA
CTTGAGCCATTGAACACTGACTACAGAAGGGCGGAAGTGCAGTTCCCAAAACATTTGTACATAGGGGAGCTCAGGCCAT
CTGTCCATATGCCAGCAGAAAATTTCAAGATCATGAAGGATGTTAAGTCAGTAGTACCTCCTGGTGGCCATTTGGACTC
ACTGTCCAAGGCGTATTGTGGCCAAGCCACAGAATGAAGAGGAACTTAGGGTCCAAGACAGCCCCAACTTGGATAAATT
GCATAACCGCCACCACATGGGCTACTGAAGATTGGGTTTAAACTTGGCGTTGTCTTCTCCCGGACTTGCCAATGATGCA
GCGACCCATATGATAGACTGGGATCGAAGAGTGTGGGGTGGAGAAACCTTGGAAAGGACGTCTCTGAATCCAAGGCCTC
CCTAGAACAAAACGAATCCAAAGCCTGCCTGGAACAAAGACTGAGTCTTTGCTCTGAATGGACAGGCTTGTCCTGGCTC
GGCCGAGACTTGTCAGGGGATCCCTAGCTTGGTCCTGAACCTGGAAGAGGCACCCGGTACCTGTTAGGCTGTGGCTGGG
AAGTGGCTCTCATTGAGTAGAGAGAGGGAGAGAAGTGGTCCCCACATGGCCTGAGGCCTGACCCACTGGACTGGGGGCC
CAGCTTCTCCCAATTCTTCAGAATCATAATAGGTTTCTCCACCAACGAAATGAACCAATTCAAGCCAACTCAAAAGTAT
AAAGGCGGGCTTATTAAGAGGCAGCTCTCCAATCTCATAGGTGGAGGTCAGTGAAGACACACATCAGTCAGGGCTAAAG
CAAGGGGAGTTTAGGGGCACAGGGAGATGATGTGTGAGCTAAGAGCTGGGATTATGCCTGTATAAGGTAAAGCATATTA
GCCCAGAATTTTCTTCTTGCAGGTAGGGTTGGAGGGAAAAGAGCAAACAGGGTTTCTTAGCTTATGCAAGGGATAACCA
GGGGCTCTAACCAAATAGTAACCATGTGTCCCAAGGAGAGAGTGTTAGGATTAGGCATTGGGGGTACCTTGGTGGGTCT
GGGCCAAGGTGTGCTGCTAAGAGATCACTCTACATAGCAGACCTCACACACAAGAGGTTTATGGGAGAGGGGAGGTAAA
AGGCCATCTTGGAGTTGAGATATAAGAAACAGACAGAGAGACTGGGAAACAGAATTAACAAGACACACAGAGAGAGGAA
GATCAGGGATGGCTTGAGACTTAAAGGGTGTGTGTGTGTCACATAGGGGAAATAAGCAGCCATAGGACGGTAGAAATGC
ACCACACCTGGCCACAGCGGGCAATGACATAAGCTACTAGCTCTGAGTTGCTGAAGGACAAGCTGGTGGGGCTCCTGAT
TTATAGTATTCCTCCTTCTTGTTTATTATAAAAGGCAAGAAAAGAGGAAGGGGTCGTGGTAAGGTGGCAATGGGGGTGC
CTATGCTTTTTAGACTGCTCCCTGCTATCTCAGAGCATGGATATCTTTGGGGACCCAAGAAAACTGGGGTGCTGGCCAA
CTCCTGAGAGTGCTGGCCATTTCCCTAGCTGTGTAGGCTCTGTGGGACCATCTGGGGCTAGCAAAGTGCAGGCCTAGTT
GAAGCAGTCTGTGAGGCTGGACCACCTGGGGCCAGCCACTTTCAAGCTATCCTAGATGCTGACTTTGAGTTAACATGGG
GACCTGGCAGGATGCTGGGACAGATACATGCAGGAGAAAATCTTTTCTTAGGTGTACATTTGAAACTTTTAGACCCAGG
GTCCTGGTAGTTGGGAGAGGGGAGTCCCCAAAACCAAGGAAAGGGGATCCCACCGTCAGAAATAAGTGGACAGTAGGCA
GGTAGGGGAACAGGCTGTTGACTGACAGTACTTATCTGGAGTCCATTCACCAGGAGAGAAGGATTCATCCTATTCCCTG
AAGCTTACGAGAACTTAAATTTACAAAAAGAAAGAAGTTTTAGGCATGTTACCATTGCCACTGTTTGTAATCTACATTG
AAATCCATATTGTAGAGAACAGACTCGCCTTTCTGTCATAGTAGAAGCTCAGGATATGATTCTGGGAGGACAAGCTGGG
GGAGGGCAGGGACCTTCTGTGGAGCAGCAAGGCCAACACACACTTGATCTTGATATTCAGTACAAGTACAGACCATGAA
AGAGGGGCTTCCTCCCTCTGTCCGGAAGACTGGATGTATCTATCAGACAGACAGTTTTATCAGCGCAATAAAAAGCACC
TGTAAGTCTATACTTAGAAGACAGCCATAGGAAATCTAAAACCTTGAGCTTGTGACTGTTATAAAAGCAGTAGTGCTTT
TCCAGAGCTAGATTGATAGCTTATTAGAACTGCATTGACTCGGGTTAAAACTCTGGGTTTTTGAAGTCTGAATACAACC
ATAGCACAGCAAAGGAAAGAAATCTGAAGCATTTTTTTTTAATATATACCTTGTTACTTTCTTACGCCTTTACAAATTG
CATTTACATGCCTTAGAAACACCTCCTCAGACCCTCAGACATTTGCTTAGATCCTCAGAGCTTAAGCTTTAAACTTCAC
ACCTTTTGTTCCTACCCAGCCGTCGACTGAGAACAGTCCTCGGAAAGCAGAAATAGTGGAAAGTTACACTGGACTCTAT
CTTGTGAGTTTGACTCTTTTCTGAGTCTGGTAACAAGGCACACTGGGTCTCAACCCAGTAGGAGCTCTAGGACAGTGAA
GCCTGGGGCCTGATTTTTACACCTGAAGACAACTGAGCAGCGACTGAAGCCTTGGTTGCTGCTGTTAACAATTTACCAA
GCTGTCACTAGCCGAGTCCTTGGTGCCAGAGCTCTGAAAAGGATAGATTTCATTTGAGTTAAGTAGGAAGTAAGGCCAC
GTAGCTTCCGAATCAATTAAAAATGACAGAGACTTACCAGCTACCTGGACAGTCACCCTAGGGTGTCTCTGTGGTCATT
GGGGACAGAGGTCTCAGGGCTGTGTCAGACTTCACCTGCCAGGCTCAGAAGGTCCTGACAGATTGTCCTGTGGAGTAAG
AACTTCGGGGGGCACTTGCCTACCTTGTCTAGGCAAAGGAAGGCAATCTGCTCCCTAGTGTCTTACTTGTCCTCAGGTT
GGACAGTAGCATCCTAGCAGCAGTAGAAGTGAGGGGCAGTTTTGGGCCCAGTGTCCAGCCTTGCCACATTTAAAGCAAG
CTCCAAGTGAAGGTTCTTCTGTGCCCATCATCTTCTTTGAAGGAGCTTAGGGGTGCTGCCAGGAGCTGTCATAGCATGT
CTCTCTAGCATGAAAACCTTTTTTAAAATTATTATTAATTTACAATGCCATATTTTGCAGATCTCTGAAGCATTTGAGG
ACCATCTATTAGTATAAATGATTTAGTGGTCAAAGGTTCTCCGAGAGGCATCGCAAAGGGGAGTGAGGAGGTAAATGAC
ACAATTCCCATGGTGACATAAAGGGGGAGGTCCAAGGCCTAAGGTATCCCAAGGACTTAGGCACAAGTTGAACAGTGGG
TCACCTCACACAGTTCTAGGACCAGGGCTAAAAGCCCAAGGAGGCACGGGATGCCTGGTACCAGGACTTTCTAATGAAG
CCAGAAGATGAAAACCATACTCAAAAACAAAGCGTCATCCAATGGGAGTGATCAGGGGTGGGTTGCTCTGCCAGAGACT
CTGCCAGTGGACAGGAGGTATAGCTCCCAGTTGAGTATATACTTGATGTGTCTGAGGCCCTGGGTTCCAGCCTCAGTCC
GAGTAAGTAAATAAACCAACTCCAGGTGGGAAGGTCACGTAGCTGTGGGGTTCTCCCAGGCCCCTCAGCCTCCTGTAAG
GATGATGGGTGAAGTCTGATGCTGAAGTTAGGTGGCACGTCCACTGGACCTCAAGCAGTGCTAGGGACAGAGTTCACTG
CGACCTGAAGAGGCTCCACAAAGGAACCTGGCTTAGAATGAGGCCACTTAGAGGCAGAGATAGGGAGGGGGCCTGAATG
GGGCAAGGCTGTGTGCGGTACTTCTAGATGGAATCTGTTGAGGAAGGGGACAGTCTTGATTGCTGGGACCTCCATGTAT
GGTAGGGGGGCAAGGATCATCAAACCAGAGCTGATTCATGGGAAAAGACTGGATGCTAGTATGAGAGATCTTGGATGCT
GTGCCTCTGCTCCAGGCGATGGGTGTCACTGAGCCGGCTTAGGGATGCCATTCTGCTGAGCAAGGGGTGAGGTGGCTCA
GAGGGGCACCTGGGAAAGATGAGAAGACCCCAGTGAGGCCTGTGCTCGGGACCAGCAGTGAAAGCCAGGACCTGGAGTG
TCCTGGGATCAGTGGGGACCACCACAGAACCACAGTTAGCAGAGACTAGCATCACGGTGGTCTAGGAAGCGTTTGTAGA
GATGTAGGGAGCAGAGGAAAAGGACCATGGGATGGGAGAGGGCAAAGCTGAGCTGAGGGGGAGGGGGAAGGAAGCAAGT
TTACTTATGTGTACTCTCCAGTGGCATCAGGCATGTGGGGGTAAGTACACACACACATACCAGCAAGGTAGCACTGTAT
GTTTTTCTGTAAGCACACATACAAGATAGAAGGTAGGGAGAAAAATCAAACTGAAAATATATACCGTGCTGTGTGATTG
ACAGAAAGCATAAAATACTGGCTGCAAGCCAAGTAAAACAGCCAAGGCAAAGAGACAATGAAATTCAGGGTATGTTATG
CCAAAAGAACCTCATATGTTACCCCCAAAGACGGGACACAGAGACATCCTTAGAATGCTAGCAGCAAGCTCCATTGCCA
CCCCACCCCACTCAGCCCAGCCAAGGATCTGGGTGGGGCTGGAGAGCATCCAAGGGCTTGGTGCACAGTAGTGGTGAGA
TGTCCTGGCTGGAGGCAACGCTAAAGTCTTGTACAATCAGTTAGTCTGGGTTTGTAGCCAGAGATGATGGGAAGGTACA
GGCTGCCCTGCACTTTGCATCCGCTCCTCTGGTGTTCTTGGCAAGTGTGATTCAGCCCCCAGCCCCCAGAATGCTGTAG
AAAGGGGCTGTTTAGTCCGTGGATGCAGCTTCTCACTGTAACGGCCTTGAGAAGTAAGGACCACCACCTCACATCCTCC
TGTACTTTATGATATGGACACTCAATTCTGATCCTGGCCAGGGGACTCTTCCCTCCCAGGCGCTTTGGGGGAGGGCCAG
CCGTTAACCAGCTGGTTGCTAACCATCTATTGCTAGATGCTGGAGTTGAAGCACAGTTTATCTGTCACCGGAGGAAGCT
TCTAGCCAGACTCTGCCTGGCCTCTCTCTGTGTGGTGTTGATAGTGAGATCACAGCCCGGTTCACACTGGGTCGACAGT
TTTGAGGGGCGGGGTGCAGGCAGGTAGAGACAGGAGAGAAACGGGTGGGAAGCAGTTCCTTCCTACTCCAGCCGAGCGA
AGAGCTGAGGCTGTGGGTTTGCAGAGAGAGAGCATGGCGTCTTGGTGAGCGGCACTTCTTCCTTAGCCCGCCTCCTACA
GTGATTGACATCAAGCTGGACAAGCCTCAGGAGCCCCCGGCCAGCGAGGGCGGCTGCTCCTGTTAATGCACCGCCCACT
CGGCTTCCTCAGCGCCACTCACTTGTTTTGCCTCCTATAGCTTCCTAACGGGGTGGGAACTGAGTTTATCAAGACTGGA
GGATCTTTCTTTTCTCTCTCTCTCTCTCTCTAGGAGTAGGGTGGGACATGGGGAGGGAGGCTGGGCATCAGGGACCACG
TCACTCTTAACAGCTGTTACCTAAACAACTATTTTTTGGTTTGGTTGTAATATATTGTACTTTATTAAGATTGCCAAAA
ACTGTTAAAATTTAAAAAAAAAATTTAAATCATGTGTATACAACTTTTTGCCAGATTAGAAAAGTAGTCATTTTTATTT
GAAAGAAGTGCTTTTTTGTTTGTTTGTTTTTGTATATTTGTAAACTTACAGAGAACCTCTCCACACACTTCTTCCTTCC
TGTTCTCTTGGAACTGTGTGTCACCTCTATTTTCCTCCTACCCCCGCCCCCCCCAGCCCAGAAAATTAAAGCAATTAAC
TGAACAGTAGTAATTACATTCTAGTTCTGGGTCACATGGCCCAAACCAACCAAACCCGCACTCCACAGTCAAATCCAAC
TTTAATCTAACAAAACAGCCTCTGAGGGGGTATTGTCGAGTTCTTCCCTGGCAGAGTGGGACACATTGGCAGGCCCCTG
AGAGGAGATGAAGGCCCGTGGACTGTAGATTCAGCAGCAGCTCCTTGGCTGCTTCCGCCTCTTCTCCTTCCCTGAAAAA
AATAACCATAGAGCCAGAAAGCACAGGCAACAGTGTGTTCTCTGGGCTGACCCGGTGACAGACAGCAAGAAGTTACTGG
AAGGATGGGGTGCGCTTGGCAGCTGAGGGCTGAGGCAGTGTGTAATCTACTGGCCCAACCATTGACACACATGGTTAGG
CTGACTCGCCAACTCAATGTGTGATGCTGGGTGGCTCAGGGACACTGAAGCCTAGGGCACCTGCCATCACTGCAACTAC
CATTGAGGAAGTGAGAGGAGTCGGCCATGACTCCGGACACTCCATGTTGTCAGACCCTTGGATACTAATCTTGCCGAAC
GTTGTTCTGGAAAAGCTGATTCCTTGATGTGTCCTCACCTCTGGCGTGGTACGTTTGAAATCTTTGTATGAATACCTTT
GGGAGTGTTGGCAGCTGCACTGCACGCATCTTGGAGTCACTGTGGGGTTTCCAGAAGGCTGCTGCCCCAGGGTACTGCA
ACTTGCCCCCCAGCAGGCTGTCCTTGCTATTTCCACTCATTCCCCAGACTGAAGACCTTCAGAGTCTCTTAGCAGAGTG
CTCTTCTCAAAGCAGAATAGCTCTTATATTCTAAACGCTCCCGGACTCACTGCCCCTGTCTGCCTTTGGGCAGGCCTGG
GGCAGTGACTCTAATCAGAGCAGCCTAGCCCCATAGCATTGGCTCTTCTTTCAGACTGCTGGTGGGCAGAGTGAATTGC
CTCCACAAGGCAGAACTCCAACAAGGAGGTTGGGATGAATTATGGAATCTCTCAAAGAATGAGTTCAAAATGGCAGCCT
GTGTGGCAGGGCTGATTCTGTGAGTGGGCCCGTCATGCGACTCCATCCTGGGAGACTGTGTCAAAGCCGGTTCTGATGA
TGAAGAGAGGAAGCAGGACCGCCCTGGGTACCTCCTCCAGAAGGCAAGGACCAATGTACGAGCATAATCGAAACGTCAC
GTGTTTAGTTTTTATATGTGTGTACATAATTATACATAGAGATAGACTTGTGTATACACCCTTCGCGCGCACACATACA
CACACTCACATCCTCACATATACATGTGCTCAGTGTATATCCTCGTCTCTCCCGCTCACAACTTGGAACTTACATTGTG
ATTCCGGTTTTTGGAAGTTCAAAAGTATGCAGTGTATTGAAATAAGAAATAACTCAATATTGTCAAATAACTTGTGGTT
GTCTTGCCTTCAATAAAATAGTGCATAGATGTGTTAAACTACAAGCAAAAGTAGTCCACAGAACTACCTTTACCACTGC
TTTTTGTACCTGTGTGCACAGGTGTGCATGCCGTCGTGGGCCAGTGTCCCCCACTGTGGACACCCCACCCATGTGGTGC
TTGTTGGAAGAAAGCATACTTAGCCCGTGGTGACAGCAGCCTGGTTGGCAGTGAGGGGCTTTGCTGGTGGCCTCTGGCA
GAGCCCCACTGACAGCACTCTCTACTGGCTTTCACTGAATGAATGTAGGGTGAATTTTAACCTTCTGGACACTGAATAA
CCTTTCTTGTATTCCTGTAACTCGAATTAGAAAATCCGTTCACTTCTCTTGTGCACACAGTCTAAGACATCATGGCCTG
CTACTAGAAACAGCAGGTACCATGGCAGATGCCATTTCTGGTCGATCTGGCCTAAGGAGCCCTGCCTTCCTAGCTTGTA
CACAAAACCCCCTCCTTGAGTTTAGACTCTCCCAGCCCTGCGGCCCCTGAGAATTAGGGTTCTGCCCCATTGCTCCCCC
ACTCCAGAGGTGCCCTGGCCTTATTTCCTCAGCCAAGCTACCCATCTCCTTCCCAAAAGGCAATCAAGGCAGCCAGGCC
CCAACAGAACTAACTGTAGTGGACCTGGGCTTTCCCCCTGACCGCCACCACCATCCTACTTGTACGTAGGATCCAATCA
GGTTCTCATAAAGACATCCTGAAATTATCTGTGCCCCCATCTTTGCTAAATTCAGATCAGTCAGAGGACAAAATGGAGG
GTTCCTCTTAGGGCATGATCCCAATGTCGTAATTCAGTTCCTCTCTCTGGACACTGCCTGGCCTGGCAGATGGGCATGG
CCCTCCTCCTTTTCTTTAAGAGACTTGAAAGAGAGTTGTCAGCAAAACTGTGAGCAGTGCAGAGATTGTGAAAGGGCAG
GTAGTGTAAGTAGTATTGTTGTGGAGTCAGAGGTTAGAATGCTGGGTGGTTAGAGCCTTGCTGCTGCCAGTCACTGCTC
CCCGTTGCCATCTTTAAACATTTCGCAATATCTCAAGATCCAGGAAGGCATGGCCATGTCAGTTCAATGTCTCAGGCAA
CAAAGACAAGGGTTGCAAGGTGGTAGCTAGGAATATAGACGCCTGGGCCTAGTCTCTGTAACCCAGTGGCTAACCAGTG
TCAGAGATCTAAGCAGGGCTGGCTTCAGCCTCCTGTAGGCAGGGCAGTTTATCCCTTTTCCATATGGATGGGGGAGAGG
CTGTCAGCAAAGTGGCTGAAGAGAGCTAAGTTAGAGCTACCCAAGAGCCAGCCAGCAGGAGTCTGTGTCAAATGCTCAG
GGCTCGATGCTCACTAGTTTCTAAACTACCTCTCCCCCAAGAATGGGAGGCACCATCACCTTGCCACAGCACTGGGAGC
CAAACCCTTCCTGTTTCACGGGTGGGAGAACCAGAACAGAAAAGGTCACCTGCAAGCCAGAGAGGATCAGAACTAGTGT
TGGCATTCTTGATCCGTTGCTGACCTTGCGGTGTCCAGACAGGACTGCTGTGCTAGCGTCCCTTAAGCCATCTCTACTC
CATGGCTAGTACCTAAGGGTCTTCCCAGCCTGGCTAGCTAGAGAGAAGATAGGTGCATTTTGGGCTCTCACTGTAAAGA
AGAGTGGTCCCCAAATCTACTTGCATTGTCTTGTGTACACACACACACACACACACACACACACCCCAGTAGTCAGGGT
ACCCTTTTAATTTTTGTTTGAAATCAACTTCTTCACTGTTTGGAAATCTTGCAGTTTCAATGTGCACACTCCTTGGACT
CCAGACCCTGTCTGATTGATACTCTCTGCAGGGTATAATTATCAGGAAATCCACGCAGCGGGAGTTCAGATGGGCCCTT
CAGGAGGCAGGAAGCCTCCATAGCAACGAAGGCCAGCCTGCCATTGTGATGTGTTGACTTCTCACCTCACCTGTCCTTT
AAAGTGAGGAGCAGAGGGCGGCATTTCTATCTGTACTGGGCTCAGAAGGCACAGTGGCCATATTGACTTTACATTCAAC
TGTGTAATATGTGGAAAAGACTCAGCATGTTGGTCAAACAATAAACTGTCCTAATTCTCGACACAAAGCTCCTTGTTGA
CTCTTTCTAAGTGGTGTCGTTATATATTGGGGCTTAAAAGCGGGTTTCCTCAAAAGCCCATCCTCTCCTGTCTGGGTGG
TCTATCACCTCCACAGAGCCCTTCCCTCTTGTCATTGCTCACCTGGGCTTTCCACCAGTTCTAAAAGAAAACTATGGAC
TAGTGAGATTGTGTTCACAAGCCTTCCTTACCTGTCTGGGAAGCTCCGGGAGGGACCAGGACTTGGGAGTGTATTTACC
ATCTGTCTTAGTCAGGGTTTCTATTCCTGCACAAACATGACCAAGAAGCAAGCTGGGGAGGAAAGGGCTTATTCGGCTT
ACCCTTCCATACTGCTGTTCATCACCAAAGGAAGTCAGGACTGGAACTCAAGCAGGTCAGGGAGCAGGAGCTGATGCAG
AGGCCATGGAGGGATGTTCTTTACTGGCTTGCTTCTCCTGGCTTGCTCAGCCTGCTCTCTTATAGAACCCAAGACTACC
AGGCCAGAGATGGTCCCACCCACAAGGGGCCTTTCCCCCTTGATCACTAATTGAGGAAATGCCTTAGAGTTGGATCTCA
TGGAGGCATTTCCTCAACTGAAGCTCCTTTCTCTGTGATAACTCCAGCTGTGTCAAGTTGACACAAAACTAGCCAGTAC
ACCGTCACTCCCACACTCTACTTGACATGGCCAGGGATTAGGAGGACAGAATGCAGGGAGGAACTAGGTTTCCTATCCT
CTTGGTTTCCAGACTATCATTTAAAAGGTAAGAACGGAAGAGTAATTCTGATTACCTTTAAAATGACGCCAACTGTGAC
CACCCGCAGCTTCTCCCACACCAGTTAGGGAACAGTCTCTGAGAGGTTCCTCAGATCTGGACAACCTCCAACCTAGCTG
TTGCCCTCAGCCAAGCTGGCTCTCAGAACAATTCCTCAGAGTGCTGGAAGTGCCACTCTTCAAGTATAATAATAAACGA
TTCTGAAAATTCTGGAACACTCCAGTTTGCCGTAATTAGAAGCTCTGACTGTCCCTGCCGCTGCCTTAGATGGGCATCT
AGTCTTAAGGTGCGAGGCGAGCAGGTGCAGGAATGGTGAAGGGTAGAAGGCTGAGAGCCTGAGAAAATGACTATGTGGC
CTAGAGAGTAAAAACAGCCAGTCCTTCCCCAGCACAGTAGTGGCGGGGCCACAAGGCTTCACGATACTGAGAGAATGGT
TGTTCTCTTCAAGTGAGGTGTGAGCACGGCTGCTCAAAGTTCCGGAAGACTTGAGGAGGTACTCCAGACGGGTACACGA
AATGACACTGACTGCTCTGCAATAAAGTGCAATATGGCAAAGTCAGGGCTACTGGGTGACTTTCCTAATCACTGCAAGA
GTGGCCACTGTTTGTTAAGCTCAGATTTATAGATTTCAGGGAAACCTTGTAGCCTTGAAAAATACACACACACACACAC
ACACACACACACACACACACACACACACACACACACACACCCTGAATGCGTACACAAATACACATCTTTTTTCCAGGTC
AGTAGAACTAGGTATCTACAGGAAGAGAAACAGCCTACCCAGGACCCCATGTAGCACAGGGCAGCGGGCAGCAGAGACG
GGGTAAGAGTGGACTGAGCCAGCTGAACTTTGGCAAGTCTTTAAGAAGCAGATGGAAGCAGCAGGCTCTGGATGGAATG
ACGCCAGCAGGACACGGTTGTAGTTGCCGGTTAACCTTACTCAGATGGACCCGGTGGCTGAGGCAGCAGCACATTCCTT
GCTACCTGCACCAACGTCCTGGGTTCCCAAATGAAAGCCTGATATCTTAAAATGCCTCGAGCAGCTCAATGGCTGGGCT
ACTCCCAAGCCTCCACAGAGCTAACACCTCCCCTGATATTCATGAGTTATTGCTTATTAAAATCTGTGTTTTATCTTTG
CTGCCCCCTAGACCCAGTTGGGGGTTGGGGGGCCTGGGGCCACTCTTCCCCAGCTCTCACGTGATTGGTATGCTCTTTT
CTGCACCTCAGGCCTGGGCCTTACTTCTTTCCTAGCATGGCCACTATATCTTCCTCCTCCTCCCATGTTCAAAGAACCC
AGCTGCCTGCAATGGCATAGACTTTTTAAATTTCCTTTAAAGCAAATACCATCATTTCTCCTTTCTACCCTTAAAAACA
ATTAAATCGATTTTTATCCATTCTCCCTTCTGTGGAGATTAACATCTCTGTGGTTACATATGTCACCTTTCTCGTATAA
CTTGAATTAACGTCTTTTTTATGTATTTAACTTTAATTAAAATTTCCATTTTTGTATGGACCTATATTTAGGCTTATCT
TTTTAAAACAATTTTATTTTTTATTTAGGCTTGTCTTTAGCCTGTCTTGTCTCAGCAGAAGCTTTTAGTATCTGTGGCT
TAAGCTCTCTTTGTAGCAGCCCAGAACAGGAGTTTCTGGCTGAATCTTTAGCCCACTGCCCATGACCCTGGGTCAGCCT
GGGTGTGTCCTAGCCTTGAGCCACAAGCCCCAGAATCATGTGTGCCAAACTAGCATGAAGCAAAACCTTAGTTTTGAAC
CTTGGGGAGGAACCACATGACTCAGCTCAGCTCAGAAACATTCTCCTTGCCTCATAATCTTAAAGGGAGGCATGTGTAT
TCTTGGGAATCTCTAACTTTTAAAAGTGAGTGTAAGTCCCACATTGGGTGACACCGGTTGTCAATTTTACTCAAATCTA
CGAGCGCGCTTGATACCTACACCAACATTCTGGGTTCCCAAATAAAAGACACACACATAGCCTTATATTTTATTGTGCC
TGAAATAGCTCAATGGCTGGGCCACTGCCTCCCCTCCCCTCTGATAGAACAGCTCACGTCAGGAGGTGCTGGAGGCCCT
GGGAAGCAGTGGCAGGGGCCAAGGCCTCCCGAGAGACCACAAGGCACCTAGCAGTGAGAAGCTGCTTCTGCCGTGGCTC
CATGGAAGAGTCCAGACATAGAATCTTTGCTGCGGCTGCCTGTTTAGAGCTGGCTTTTACAGCAGCCCACAGCAAAGCC
AGACCCATGCGTTCCAGGTAGGCATTCTACTAACCTGGTTCCCAGCCCTGCACAGCTTACCTTTTGCTCAGGACACAGG
CTATAAATAAATAAATAACATTTATCCTTGCTCTCCATCCTTGTCACTTTTTTGATGTACTGATTCTCCCACTAAGTAA
AATCACTTTATTATGCAGACAAAAGGGAGGCCAGCGAACACATTTTCTAACTGAAAGGTTTTTTTGATGAGCACCTACT
GTGATGTGAACGTCCTTCATCGTGCCCTTCCCATTTGTCCGTGACCAACATGAACAGCAGTGTGCAAAGGGCAGTAAGA
ATCCTAGCAGCAATACTGCCTCCCAGTGAGAACTGAGTCATCTCATGGCTTATCTGTCCTGTGCTGTGCTGTATCTGTC
TCATACACCATAAGCAAGGCCAGTAGATATTTTAGGAGTGGGGAGCTGAGTACTTGATGGGAATCATGGTCTGGGACTC
TATTATCAGCCCAGGTGACCCACAGGGAGGCCATGTGTCAGCAGGCAGAGGCTTGGGCTGGTCTGCACAGAGAACACCT
GTGTGTTTATCCTGTCCCTCTCACTGGTCCAGGCCAGCCCAGAGCGCCACACATATTCCCTGAGCACTGCACCTGAGTC
AATGCTCTGCCTGTCTCTTTAGTGTGGCTTTAGTGAAGCCTGACTCTGAGTGTGAGACGAGACCAAGAGTCTCATATAT
GCAGCGATCTGTAGGCTCAGTAACCTGGAGCTGGGGTTCAATTCTAGCTCAGGTTCCCACTTGTACTCATCATTTGACT
CAATGCTTTCAGTATAGTCATTTATCAAGATACCAGTCATTTATAAAAGCAATGAGAACACAAATGTTGAAGACAGCAT
CACTTTAAAAAGGTAACATTTTTTAAAGCATCATGAATAAATATGTATTTAAAATGCACAGTGGACATCTTCAGTTTGT
ATTGTTCCATAGCACTCTAGCTGGGGTGACACAACAGGAGCTGATCAAGTAGCACAGGGGACAGCCTGGGCTCACGCAC
GCCCGAGGCAGCAGTGTGCTGTCTCCCTGCTCTCCCCAGTGGAGCCTCTAGAACACTAACCTAGGAGTGTCTGGGGATT
TGGCTCCTAGAGCCACCTTGTGAGGGTGACTCTTCGGGGTTGCGGCAGCTTTTGAACCTAGGGTGGTGTGGGGATGGTC
ACCGAGCCCGTGAAGGACACACTGTCAGCTAGGGTGGTGTGGGATGGTCACCGAGCCCATGAAGGACACACTGTCAGCT
AGGGTGGTGTGGGATGGTCACCGAGCCTGTGAAGGACACACTGTCAGCTAGGGTGGTGTGGGATGGTCACCGAGCCCGT
GAAGGACACACTGTCAGCTAGGGTGGTGTGGGATGGTCACCGAGCCCGTGAAGGACACACTGTCAGCTAGGGTGGTGTG
GGATGGTCACTGAGCCCGTGAAGGACACACTGTCAGCTGTGCCTGTGGCTTTGAAGCTGTCTTCACTGTAGACCTTCAC
ACCCCAGACTCCACTAAGAACTGACACTGTATTATGCCTACACCAGGGGCAACTGCAAGTTCACTTATTCCACAAGCAG
GCAGATCTGGTCCCAGGAAAAGGGACCAACACAGAGGCAGGAGACAAGGACTTCTCAGATGCGACCCTACTCTCAAAGC
GATGCCCTCTCTGTTATGTCTCACACTAACATGATGCTACACCTAACACCTAGCAAAGGGAGAAAGAATATGGGAATTA
AGAAAGGACTAGCTGAGAAAGGAGCTGACGGGGGTGGGGTGGGGGTGGTACAATTGTTTCAAGAACTCAAGAAATGCTT
CAACGACATGCCCCCTCTTTATCAGTCCACACCACCACCGCCCTTTCTCAAACTGCAGGGTCAGTCTCACATGACCAAC
GCTTGTGGCCAGCGATGCTCCTCAGGCAATCTTGGCCAGCCACTTCTCCAAGTCCTGGATGTCAGCAGAGCCAGTGTCC
CCTCGTCCACCCTTGGCACTACACTCCAGGAACTCCACTTTGAGTGGCAACTGGGAAAATTCAAATTCTTTGCCTTTCT
TCCCCAACTGAGCAGGTGCGGTGCTGGAACTGTCCAGTGTGCTGGGAGCCGCGGACCGGGTGACTCGTAAGGTGTTGCT
GGGGGCAAAGGGAGAAAAGTCAGACGCAACAAAGCCCAAAATCCTATGAGCTCCTAAATCAAAGGGATAGCACCAGTCG
GTATTGACACTGCTGGCTTGTCCTTTATGTCCATGGATCACTGTTTCAGACAGACACCAGTGGGTTAGGAGACCCAGGT
CACACCAGCTCTTTGAAGCCTCTACCTACTTCTAAGCTCTCCTGAATGGCCCCACTGTATTATAAAACTCATGGTTTTG
AAACTTCAGAAAGGGTAGATAGGGAGCCTTGGCTATGAAGCTCACACATCCACAGACTTCCGTCTCATCTGTCCTGTAT
CTGTAAGCAGGAAAACCTTCCCAAACTGTCACTGTGCTGCCAGCCGCTGGCCCTGTAGAGCACACAGGCAGCATGCTTG
CTGCCTTCCTGCTGGGGACATTTCATAAATACTACATCTGACTAAATTTACAAGCAGGGCATGGTGCCACACACCTTTC
CCTAATTTGTGAAAAACCTTAAATGGCTATAAACACCCACGTCTTTTATAATTCTAATAATCCCTTTAACACATATTCC
ATACTACCCCAGCTACAACTCTGATCACTCGATCCTAAGCTGCGAATGGACTCCGACGGTCAGGACGCCACCCGGAGCA
CAGCCCCACTTACAGCTCCTTCTCAAGCTGCTGCTGGATCAACTTCGCTGATTTTGCCATTGCGATATCTGGGAAGGAA
AAGAAGAGCATTATTCTAGTTTAAACCAGGATCTGTCTCCTCCCTGGACTTCCAGCACAGAAAGAAAATTCCCAATTAT
ACCAACTAGTTCACCCCACATTCCTTTGTTGAAACACTTTTTATCATTATTCCTTTCTGTCACAGTAAATGCTGTATTT
GCAGGTTTTTAAAAAATGTTTTAAAACATAAAATACAACAAAATATTGTTTTAGTGCTAATGACCTTAAAACTAACCAA
GCTTAAGTTTCCCACACCTTACAAGCCCTTCAATATGTAACCTGTCCTGTCCATTGAAAGAAAGCAAGCATACGTGAAC
CATGACTGACATCTGAGGAGAACTTTCAACACAACACCACATTAACCCTTAAGAAAAGATGATGGCTAGGGCTAGAGAT
GGCCCAGGGGTTACAGCACTGGCTGCTCTTCCAGAGGACCAGGGTTCAAGTACCAGAACCTGCATGGCCAACTCACAAC
TGTCCCAGCAGATCCAAAGCCCTCTTCTAATCTTCTCAGGCACCGGGTATGCCTGAGGTTCAGCCCTGCACACCCCCCC
CCCCCCCCCGCCCCACATACACACAAAACAAAAATACTTTTACAAAAGATGCTAAGAGCTGCACCATTCATCAGGTATT
AGGAAATGTAGCCGTGAAGGAGTTCCCGAGGACCAACTCCTGACGTTACTCCGCCTGGCCACAGAGCTGATGCCCAGCC
AACCTGACTGTGAGCGCTGGGGTAAGAGGGAACAGTGGCGCAGCCTGTCTGGAAGGAGAGGCCCAATAATCCCCCCAGA
ACCGATAGTACTCGGTCTCACAAATTAACATTTGGAAAAGTAAGATAATTTATCTTTAAAAAGCACTGTATCATGAAAA
AGATATAATATCAACAGAGCCCCCAGCTGCCAGTCCTTGGGACAACCTGGTAAAAGACCAGGAAAGTAATAGAAACAAT
TTTCAGATTTAATCCCTTAATTAATGCTTAATTCCAGCCCTTTCCCCATGCTCAGAAATAACAACGGCGGGTAAATGAA
CTAATTCCCAACAGCAATCCTCAACGGTTGGCTGCCTTTGCCAGGAAGCACCTTGCTTATTGCAGGCTATTAATAAGGA
CGGTGAGTTCTTCAGAGCCATGCTGTCGATGAGGACCTGGTACAGAAATTCGGCCACATCTTTCACCTCCCGCTGGAAC
GCCGCACTGTCCACCACAAACACCACAGCCCTGCAGGGACAGTTAAAGGGAAGGACACATGAGCACACACGGCAGGCAG
GAGCCACAAGACAGACCCCTGGTTTCTCCACTCAGCCCTCCACCACAGCACACATGCACGCATGCATGCTTTGATTTAA
GTAAAAAAAGAAAACAAAAACAAAACATGAAGAAGCTAAAAGGACACAAGAAATGACTTGGGCTTTCAAGGGTCAGAAA
ACCATCTGACAGATCTAGGCGATGAACACAGGGCAGAATGTACCCACAGTGGGAGAACAAAGAGGGAGACAAAAGAAAG
ATGTAAAAATCTCGAGAAGAGCACAAAGGCAGGGTGAACGGGGGAGGCCAGGCCAGAGGGGAAGGGATGAGGACACTGC
CCTGTGAAGCTTGTGCTGTGAAGACTACTGGGGCCTAAGTGGAAGACAGAACACTCTTGAGGGAACTCAAGGATTGTGA
CTATGTGGGACCTGACCTCAGACACAGCAGTGACACCATCAAGTACCAGAGGTACTGCACGGGAGCCTCAGACATCAGG
TCCTGGGGCAGAGAATGTGCAGTGGCTAACAGGGTCTGGACAACTGGACCAAACCTTACCCAAAGCCTCCAACTCTAAG
GCAGTGAAATGGAGGCTGTGGTTAGAAAATAGTTTTAAAGACGTTAAAACACCCGGTCTCCAATGGCTGTAGCTATGGG
GCATAAGTGGACACTCCCTTGGAATAAGAGCCCTAAGCAAGAGATTATATAAAAATTGGGGGCTGGAGAGATGGCTCAG
AGTTCTTCCAGAGTCCTGAGTTCAATTCCCAGCAACTACATGGTGGCTCCCAACCATCTGTAATGGGATCCGATGCCTT
CTTCTAGTGTGTCTGAAGACAGCAACAGTGTAGTCATACAAATAAAATAAATAAAACTTTAAGGAAAAAAAAAGTTAAG
AAAACAATTTTGGACCATATTCATCATCAACCCCCGATGGTGCTAACAAATGAGGAGAGCGGTGTGGAGCTGGCCGCTC
ACGCCCTACACTAACAAAGACTACCAGTCTCATCGATGGCACCCATTCTCTTAGAGGAGGAGTTCTGGATCAAAACAGA
AATTAAAATATGCAATTATAAAAGAGATTAACCAGCACCCCCAAAACTGGACTTTAGAGCAATCTACAAAGCACATGTT
TATGGGCTGGACATGTAGCTCAGTTAGCAGAGTGCTTGCATAGTAGAATATATATATATACATATGGATGTATATGTAT
ATGTATATGTGTATGTATATGTATCCATCCCATCCCACCCAGCATTGAACCAAAGTGATCACCTGTAATCCTAGCACTG
AGGAGGTAGAGGCAGGAGATCAAAAACTCAAGTTCATTTTTGGTTGAACAGTGAGTTCAAGGTCAGGCTGAGCTACATG
AAATTCTGCCTCCAAAAAAAATCATAATGTATGTTTAAAATTATTTTAGAGAATAATACTAGAAGCAAGTAAAAAGAAA
TGGGTGGAGTTTTAAAGGGTATTTTAAAAAAGAACACATTATAAAAATAGTAAATAAAATTAGCAACACAGTGGAAACT
AAACAGGTTCTCTCAGCTGAAAAGAACACGGCAAACTAGGCAGCGGATCTGAGGGGCTAACACTCTGAGGGGCAACACT
CGAGAGAGAAGGATGTGTCAGATAAACTACGCAAGAGCTAGGGCAGAGAGAACGGCCCATGCTGTGACAAATGAGGCAG
CAGGCACAGGGTCGGATGAATCTGGCTTGATGTTTAGGCTAATCTTTCTCTTGAGGCTCTTCTACCTGTAAATATCCAC
TTGTGCCTTGTAGCGGCGAGGCATGGAGACCGGGTGTTTCAGTTTCTTTGGAATTCCGTTCCAGCTACGGTCTCCATTA
GATTACCTTTGAGTGGCAAGCTGTGCACAGGGTGACTGGTCCAGCTGCCCCCCAGGGCCTGCACTTAACAGATGATAAC
ACCCTTCTGTGTAGGGTGCTACACTTGAATTCCAAATACCAGAAACCCAAAGACAGATGCCTCAGAGGGAAATGACAGT
TACACTGGCACTCTGTAAGAGCAGATGTGGAGTCGGCGGTCTCCAGGGACCACTCGGAGCTGCTACCCAGGACCTCACA
TGCAACTAAGACCAAGGACCATGAAAAAAAAAGAGGCCAAATAAAGGTTGCTTTCCGTGAAAGACAGCCAACAACTTCA
CGACACACTCATCCTCTAGAAACATTTCTAGCAGATACTCTGAGGAAAACAGAACCTGAAAGGAAGGAGCAAAATTTGG
CAAAGAGCAAAGAAATCAAACATCAGGGAGCCTCAAAACACAGGTGCTTGTCATGGTTTATAAAAAAGAAATGGGTCCT
GGTTAGATACTGTTTTTTGTTTTTTAAAATGGGGCAGGAATCCACAGAAAGAAAATATAATATACTTTGAAATCCCTAG
ATGGAAAAACAATCTTAGGTCACAAGGAAGAATTTTTTTTTTAAATGTCACATTGGGGTCATGGTAAGAGTTCTTGTTG
CTCTTGTAGAGGACCCAGGTTAGGTTCCCAGCACCCACATCAGGCAGCTCACTACTGCTTGTAACTGCAGTCCAGGGGG
CCCAGCGTCATGTTCTGGTCCTTTGGGTACCTGCACACACACAGTGCACAAACAAGAACAGGGGCATGCACCTAAATTT
TAAAAAGAAATCTTAAAAAGAAAAATGTCATATACCATAGTAAAAGTGTTTTCGATTTAACAGTTTTATGACTGAATGA
AGTTTGTTAACTTAGGTTTTTTTTTTTTTTTTTTTTTTTTGGTTTTTCAAGACAGGGTTTCTCTGTGTAGCCCTGGCTG
TCCTGGAACTCACTTTGTAAATAGATCAGGCTGGCCTCGAACTCAGAAATCCGCCTGCCTCTGCCTCCCGAGTGCTGGG
ATTAAAGGTGTGCACCATCACACCCGGCTTATTAACTTAGTTTTATCTTTATATGCTGAATATTCTATTTATCCAAAAT
ACTTGGAAAAAAGTGTAATTATACTATAAACTCTAAAATATTTGCAAGTGACAATGGAAAAAAATTTTTTAAAAAGCTG
TTCTTGAGACACTCTGACAAATTAATACAAATACAAATTTTCCTTACTTTTCTTAACAATGTTAGACAGCAAACATGAA
GAAAGCATTTAAAACTACAACATACAAATTTTAGGAAAGAATTTAAAAATCAATGACATGAGGACTTACACTAAAAATA
GTTCTTGAGGTATCAATTTAGTGTGATTTAAGAACAGACATGCAGCCTCTAGCTTTTGGCTGGGTTATACGAGGGAGAT
AGTGAGGAAGGTCTCTATACGGTTGTTCCTATGAAGTGGACAGCTCCATACCCACCAGCAGAAAAGCACCTAGTAAACT
ATTAGCTCCATAGCTGCTTGCACTACTGCAGGGGTGAGGGGACCTCTCTGTGCCACGTGGTACTGTGGCACTCTGTTCT
GCTGCACTTCTGATCCAGACTCTGGTTAGCATCTGGGCCAGACCCAGGACGTGAGTACAGCTACTGGTGCTTCACAGGA
CTCAAGCCAGGCCTTTCTGAGGATACCAGCTGTGTGCTAGCCATGCAAACTAACAGTTATGCCAAAGAACTCCAGCTAG
CATGCCTGAAGGCTTAAGAATCTACAATAAGAGCCGAGCATGGTAGAGCACACTGTTAGTGCCAGGAACCAAGAGTCAG
GAGGCAGTAGCAGGCAGATCTCTGAGTTGGAGGCTAGCCTGATCAACTGAGGGAGTTCCAGGATAGCCAGGGCTACACA
GAGAAACCCTGTCTCAAACCCTGACCCCCCAAAAAGAATCTACTATGAGAGAAATATAACAAAGCGAAGCAAACAATAA
ACCCCTTTGTCCTGCTATCATCAGTAATTCTGAATGTCAGATTCGTCCCTGTGGGGTCAAAAGGCATGATTCTGCGTAG
AAAAATGGGGCAGAGTCAGGTGCTGGCGGGCTTTCCATTTTCATTTGTACTCGATTTCCAATCCAAACACGGGAGGTAA
AGCCCAATAGAGCCAAGCCTTTCTTTGGGCAACTCCCAGCAGCTCAACTTCAAGACAATTAGAAATCAATCTCTTACAG
TTTTTTGGACAATGCTAGCATTTATATGCTAAAAAAGAAACAAACAAACAAACACACATACACAAGGGAATTTCTTATT
GATGACCAAAAATAAATAAATAAATAAATAAATAAATAAATAAATAAATAAGGTGAAAAGAAGGTAAGAGGAAAGAAAG
CCTTGCTAGTTATGGTGACACATACCTGTAATCCCAACACTTGCAAATTCAAGGTTCTTTGTTTGGCTATATAGCAAGT
TCAAGGTCAACCTGCAGTACCTGAGACTCTATCTTGAAAAGCCAAAACCAAGCAGGTGGACAGGAAAGAAAAGCCACTT
AGTTTGTATAAGCCAGGAGGCTGGAATGGAAAAAGCTTACAGTTCAGGGCCAGCCTGGGCTACATGGGCTAGAGAGATA
CTGCCACCTTATCAAAACAAAAGAAAAAGCCAGGCGTGGTGGCGCACGCCTTTAATCCCAGCACTCGGGAGGCAGAGGC
AGGCGGATTTCTAAGTTCGAGGCCAGCCTGGTCTACAAAGTGAGCTCCAGGACAGCCAGGGCTATACAGAGAAACCCTG
TCTCGAACCCCCCCCCCCCCCAAAAAAAGAAAGAAAACCAAAAAGAGTATGATTAATGTTTTGAGTTTAGTATCTTCTG
GGATCCATCCTAGCCTTACAATTTCCTTTTCCAGATTGTAATAGCCTTAACATGTCATTAAATCTATTTTCAGAGTCAG
TGGCTAGAGAGATGGCTCAACAGTTCAGAGTGTGTTCTGCTCTTGCAGAGGATCTGAGTTCAGTTCCCATGTTGGGAAG
TTACAACCAACTGTAACCCCAGCTCCAGGGGATCCAACACCCTCCCTTCCTATGCTGTACCTCACACAAGTGCACATAC
CCTGCCCCACATACACATAGTTGAAGTTTAATCATGCTAGTAGAGTGACACCCCCTGCGCAATCTGAAAAGCCAGCCTT
AAGCAGGCTCCTACGTCTTCCGAGTTCTAGCACCCCAGAAATGGCCACTCCAAGGTTCATCCCATACCAGTGTCAAAAG
TCTTGGCAGAGGAACCAGCGTGAGCGTGCGCATGGACGCAGTGCAGACTTGAAGGGACCCATGTCACTTCATCCCTGAC
TTACCTAGCTGAAGACTTAAAGCGATCTAAGAGCTGAAACCTCAAGCTCTCATGCCCGGGGAGGTCGATCAGAGTCAGG
CTGTTACCCTGAGGACAGCAAGAGTGACATCACATCACCATTCGGTCCAGCTCCAGGTTCACATAGCATAGATCTTCAC
CATCAGATCAGCACACTGGAGCTGAGCTCATGAATCAAGCAAATGTTTACTGCAGGCAGCAAGTCAGATGGATTTCTGT
TGAGGATGTCTTAATAGTAAACACAGGTGAAGGAAGGGTAGTGGCTTGCCTAGTATATGTGAGGCACAGAAAAAAAGTT
TTAATCCCAGCACTCGGGAGGCAGAGGCAGAAGGATTTCTGAGTTCGAGGCCAGCCTGGTCTACAGAGTGAGTTCCAGG
ACAGCCGGAGCTATACAGAGAAACCCTGTCTCAAAAACAAACAAACAAATAAATAAATAAATTTTTTAAATTAAAAAAA
AAGGCACTAGACATTAACAAGGCTCTGGGTAATAAAAATCTAACTATGGGACGGCTTCTAAAGAGAAGTCTGGCTAGCA
GAGGCAGAAAAAGACAACTACTTTTAGCCAAATGATTGTCCTGTGTCTACATACTGCCTCCTTATACCCGAAAAAGACC
CAGTACTTACTGAACCTCATAGTAATGTTGGACAGACAGAACCTAAAAATAAAATGTCTTATACCCTGTACGTAAGTTA
CGAGGATGGGTGGGCAGGACTAAATCTGCTACCTATATTATCGAGGGCAGATCTGTTTGTTATACAGAATATGTTCTAC
GTAAAATGAAGCTACACAAAAGGTACCTACATCTTCAGACTACTCCATCCAAGAAGGTTCTTCCTTGACAAGCCACAGT
GCCCCAGTAAGCTTTGTTAACTTGACACAGCTTCCAGCCATGCAAGAGAAGCCTCAACAGAAGTCCCTCCATGAGACTG
GCCTGTGTGCATGTCTATGGGTCATTTTCTTGAGTCCAATGTGGGAGGCGCCATGTTTAGGCAGGTAGTCCGGAGCTAT
GAAGAAATCTAGCTAAGTGAACTGAAGAGTGAACCAGCAAGCAGCATTTCTGCATGGCTTCTGCTGCCAGGACCCTGCC
CTGAATTCCCTTAACAATGAACTACATCCCGGGAGTGTAAACAGATTAAATCCTTTCCTCCCCAAGTTGCTTTGGGTCA
GCAACACACATGACAGTAGAACTTAGTAAAGAAGAGTGTCAGATTCTAGAACATTCCAGATACCTCTTACCCTGTTATT
GTTGACTTTGTATATGGCAGAACTATCGGTAATGGAAGTCTGTGTGTCTCTGTACTGGCCAGTTAGCAACTGTAAACAA
AACAAAACCAAGACAGACAAATCAGGGTAAGCGAGTTACAATAACACTGAATGCAATGTAAAAAATGACATTTTCATTC
TGTGGAGAGTAAGGTCAAATGAACGAATGCTACTTGAAACAGCTGAAAACGATCATACTGCTTTATTTTTAACTTGTAG
AATAGTGATTAATTACAGCCGGGCAGTGATGGCACACGCCTTTAATCCCAGCACTCGGGAAGCAGAGGCAGGCGGATTT
TTGAGTTCGAGGCCAGCCTGGTCTACAGAGTGAGTTCCAGGACAGCCAGGGCTACACAGAGAAACCCTGTCTCGAAAAA
CCAAATAATAATAATAATTAATTACAAGCTTTATTCCTTCTTTTTGCAGTGCCATCAGAAAGTGGCACCCTGGGCCCAA
CATTATTAGTATTATTTTTAAACAACAGCAAGGGGCCAAATTACTGACCTGTAAAGATTTTAGGCACCAAAACCAACTC
AAGGCCTCAGGAGATGTTCAGTGGACATAACACTTCCTGTGTAACCTTGATGACCTAAGTTCAGCCCCTCTGAACCCAC
ATAAACCCAGATCTAGTAGTACACACATTCACAAGCCCAGTACTCCTATGGGGAAAATGGGAGGGAGACAGGACAATCT
CCAGAAATTCATACTAGCTTGCCTGGCACAGCAATGAGAGAAGACCAACACTTCTGGTCCTCTGACCTCCACAGATGTG
CCAAGGCTCACACGTACCAGCCCTTACACACATGAATGAACACACACACACAATTAAAATAATAATAGCTATTACTACT
ACTACTACTACTATTATTATTATTTATACCCTCCAAAGGAAGAGGGTAAATTCTCCATAGTAAAACATGTTCAGTTAAA
ATACACTGGCTTACATAAAACTAAGTGTTAGTAAAGGGAGGCCTCTCTAGCTTAAGACTGTCGAAGGAGGGTAATTTTT
GTCATGATTGTGATAATGCTGGATTGCTCAGAACTAGCCATGCTGGGACTATTTTAAGTCACAGAAATCAGCAAAAGTT
ATTGGGCTTCCTTTATCCTTTATTGATTGTTTAAAGAAAGAATAAGAACGCAAGCTACACTTAAGAATATATGTCTATC
CAGGCAGTGGTGGCACAGGCCTTTAATCCCAGCACTTAGGAGGCAGAGGCAGGTGGATTTCTGAGTTCAAGACCAGCCT
GGTCTACAGAGTGAGTTCCAGGACAGCCAGGGCTATACAGAGAAACCCTGTCTGGAAAAAAACAAAATCAAACAAACAA
ACAAAAAAATGTATGTCCATAACCATTATATTCTGAGTAGCCCAAGAAACTGGTAATGCATTTTTTAACAGGTGTATGT
ACACTGAAGGTTGGTTGATGTCGGGAGTCCTTAATCTTTCTCCACCTTATTTATTTACTGTTTTTGCTTTTTTTTTTTT
TTTTTTTTCCCTGACATAGGGTTTCTCTGTGCAGCTCTGGCTGTCCTGGAACTCATTCTACAGACCAAGCTGGCCTCAA
ACTTGAAGAGATCCTCCTACTTCTGCCTCCCAAGTGTTGGCTTTAAAGGCATGGACCACCACCACACAGCCAGCCACCT
TTTGTGAGGCTGTGGTCTCTCACTGAACCCGGGGAGAAAACCAACTAGCATGGGTGGTGGTGAGCCAAGGAATTCCAGG
GATCCATACCTCCTAGGCCCTGGGATTGCTGACAAGAACTTCTGGACCTGGCTTTCACGTATGTGCCACCGATCCAAAC
TCAGACCCTCATTTTTTTGCCAGGCAAGAAAACAGACTGAGTCACCTCCCCATCCCCAGACCACGTATTCTAACAGCAT
CGTGTGTGTATGTGTGTGTGTGTGTGTGTGTGTGTGTGTTTTTACAGGTTTTCCTAGAGTCTTTTTTATATAACTCCCA
GGGGGCTGGAGAGATAGCTCAGTGGTTAAGAGCACCGACTGCTCTTCCGAAGGTCCTGAGTTCAAATCCCAGCAACCAC
ATGGTGGCTCACAACCATCTGTAATGAGATCTGACTCCCTCTTCTGGTGCATCTGAAGACAGCTACAGTGTACTTAGAT
ATAATAATAAATAAATCTTAAAAAAAAAAAAAGAATTAACTCCCAGGGCTGGAGACTCAACTAAGTTGGATAAGATCCC
ATCCTGCTTCTAGCAAAGGGACCTGAATTCAATTCCCAGCACCCACATCAGGCAGCTTTGAAGACCGACTCTACAAAAT
CTGACACATCTGGCCTCTACAGGTACAGGCACTCATGTGCATATACCCACACACATACATATAATTAAAAAGAAAAAAA
AATATTTTTAAAAACATGTCCTAAACTTGCTGTACCAAATAAACTGCAAGATGACAGGGCTACGGAAATGCCTTACCCA
TCCCTCTTAAGTCTAACCGAGGTGAAAATCAAAGCATTACCCTGACAAACAGCAACGTTTTCCCAGAGTCACAGAGACC
AACGAAGAGAACAGCTCTCTGACTGCTCTTTCGGCTCCAGATGAACTTCCAGAACACTGCAGAGAAGAGAAAGGCAGCC
TTCATTTTCTAAAGACATTATTAGTAGGCATTTACGACAAAAAAACAAAGCGTCCATCTGGACTACTTTCATTGTCACA
CGGATTAGAGTCAGTTTTGTTAGGAACTCACGGAGCGCCAGCCACTATCAACTGTAAACCTTTTCACGTCTATTTCCCT
CATCCAAATGTGGAGACTAAAAGTATACACAGAATGATGGATCTGGGAAAAATTAGTGGCTTGCTACATTTGATTGGTG
GATTTCTACTCTACCTTAGTACCCCAGTATCTCAGAGCCCAGTTCAGACACAAGTGGCTCAAAGTTGGAATGAACGATG
GCGGCCGCAGCGCTCAAGCGCACAGGGCCGCCTCATGTAAGTCCTGTCCCAAGACCCAGGTCACTTCAGCGTCCCCTTT
CCACACTGCTGGGTCTGGAAGACGAGTAGTAGAAGAGGGAACGCAGCCGTCCGCCTCCCTGGCCTGCGGCGCCGAGCCA
CCCTGCCCTCGCCGTGAATGCCGGCCGCCTCACCCAGCGTCAGCAGCACCGCCAGGAGAGCCACCGCCACGGAGAGCAG
CGTCGGATCCCTCTGCTGCAGCTCCTGTCGTAAGGAGTCGAGGTAAGGCTGGAAGGCGCCACCGGCGCCATCGCCAACT
CGCCGGGTGTTCGCCGAAGCCATGAAAGAGGTGTGTAGTCTTCCCGGGCCGCCAAAGTTACGTGGCTCTAGGCTCCACG
CCGTGGTGGGGCGCGGGGCATCACGGGAGTTGTAGTCCCTTAGGGGTATCTCGGCTAAGTGCCTGATGGGAGTTGGAGT
TTTTTGGTTTCTACAACCCAGTTAGCGAGTGTATTCATATCGGCATGCTTTGAGACATGTATAGCTTTGTGGAATTTTT
TTTCTTTGCATTTTAATTTTTTATCTTTGGAGAATTTCATAAATGAATATGATGTCTTTTGGTCAAACCCACCCCTCTA
CTCCCATTTCATTTCGATTTTTGAAAGAATGATGACTTTTAGCAAAATTAACGGCTACAAAACTTCATTTGAGGGTGAG
GATTTCTACTCGGCCGTTACATGGGATGAAATTTATGAGAGATTTTACCACAGTATGATGTGACCGAAGATGTGATGGA
GTCACAGAGAATCCACAAAGACACATCAAGTCCTTTATGAGGTCAGAGAAAAGTAGCTAGAGATGGACCTGAATCTTGG
AGGATGCATTAACCTATACCGGCAACTAAAGAAAACAGAAAAAGACGCACGGAAGAGAAGCTTGAGTAAAAGCCAATGA
CATGTGGGAAACTCGGGAATTCTGTGTCCTGCATTGACACGTCCAGCACGGAAAAAAATGACTGCATTTTAAAAAGCAA
TCAGGAAAATCAGTGCTCGGGAGGATGTGGAGGTGTCGACGATAGATTGATCATTCCCACCCAAAGGAAGCAGGCCTAC
TCAACCAGGTAGGCCATTTGAGAGGCCCAGAGTAGACAACATTTTTCAGTTTCTCAAGAGGACTTATTTATAAAAGATC
TTCATGGGAACTCTTCTACTTAAGATTTCATCGGGTAATCGAAACATTTAAAAAAAAATGTGAGCTTCAATTTAAAAAT
ATCTCTGCGAGTCAGAAGTGGCCAAGCCTCGAGGGCCATTTGCAGCCTCTGTCATTAAGTAGCTGTCATTATTTTAAAG
TATTTAAGTACAGGTCTGCCTGGGAGATGACAGGAAAAGTCGATGTCACTTAAAGGCTAAACAAATTCATTAAAATGTC
GCACTGCCACATAAAACCCCAACTCCTGCAGGTACCAGGAAGAGAGGAAATGAGACATTCCCTTCCTCTCTAGTGTGGC
AGTGAGTGATTGGGTAGGAAATGAAACAAAGATGTTAAAGTTTGAGTTTGGAAGGTTCGCAATGTGATTTCGATTTCCA
TACATCCTCAAAAGGCAGAGGGCAGGTGAGAAGAAGCCCACTCGAGAGGGCCACTCCGTTGGCCTTACCATCGCCAGAT
ACTGAGTCGCTGGCTACAGGCTATGTATAAGAATGATTAGATTGTCCATGGAAGCAAAAGTGGCAGAAAGGCAGCTGGA
AAAGTTTGTAGGAATCAGACCTGGGAGTGGTTTTGTGTGCTATGCTGAAGAACTCAGAGTTTACTCATAGTATAACAAT
AAGCTGAAGGTGACATGGATAATTGGCAGATCTGTGGAGAACTGTGACTGGAGAGAGAAAAGACTGAGGCTGGGGGAAT
CAATGGGAGTCTCTTGTCGTATTTAGGCAAAAGATACTTGGAGTCTAACACAGGATAAAAGTGTGAAATCCGGGGCTAG
AGAGATGGCTCAGTGGTTAAGAGCACTGGTTGCTCTTCCAGAGGTCCTGAGTTCAGTTCCCAGCAACCACATCAGGTGG
CTCACAGCCATCTGTAATGGGATCTGATGCCCTCTTCTGGTGTGTCTGCAGACAGCTACAGTGTGCTCACATACATTAA
ATAAATAAATAAACAAACAAACACTGAAAGTGTGAAATACAGGGTAAAATTTCTGAGATATTTAGAAGTTAAAATATGT
AGGTTGTAATGACTATAAAATATATAGTTGCTGTTGTTCTATTACTGTGAAAAGACACCATGACCAATACAGAAGAAAG
CATTTAATTGGGAGCTTTTTTGTTTGTTTGTTTTTTTGAGACAGGGTTTCTCTGTGTAGCCCTGGCTGCCCTGGAACTC
ACTCCGTAGACCAGGCTGGCCTCGAACTCAGAAATCCACCTGCCTCTGCCTCCCGAGTGCTGGGATTAAAGGCATGCAC
CACCACCGCCTGGCAGAAGAAAGCATAGAAAGCATTTAATCGAGAGCTTCCTTATATTTCAGAGGCTGAGTTCATGAGT
GTCGCAGTAGGGGATGTGGCAGCAGGCAGGCATGGCACTGACACAGTAGCTAAGAGCCTACATCCTGATTCAGAGGCAG
AGAATAAGAAACTGGGCCTGGTGTGGGTGTTCAAAACTTCAAAGCTCACTCCAGTGACACACCTCCTCCAACAAGGCCA
CACCTCCTAATCCTTCCCAAACAGTTTCACCAACTGGGGACCAAGCGTTCAAATGTATGAAGCTATGGAGATGTTCTCA
TTCAAACCACAACACAGCCTAACCTACACAGTAAATTCCTTGGCTACATAGTGTGAGATTTTGTATCATCCTTTGTTTT
GGTAAATCTCCATCCCAAATATACCCCGGCGATGAAAACACAACACAGTTAATATGAATACATGCTGTGCACCTAGGTT
GGGCAGATCTACCACTACACTACCATCTTCCACATCTATGAGACCCCTTAGAACTTGCAGTTTCTCCAGGCCATGTGCT
TCTGCTCTGCTTTTCTTCTTCCTCCTCCTCTACGTCCTCTCCCTCTTCCATTTTCTTCTTCTCTCTCCCACCTTTCACT
CCACCTTCCCTTTTATCTGCCCAATCATCAGCTCTTCTTTATTTTACAAATTAAGGTGGGAAGCAGGTTTACAGGAAAT
CACCTGAGTGCTGACTCATTCCTTGTTCGCAGCCCCTCACAGGAGAATGGAATTAATATCAAATATAATTAGCCCCAGG
GCTATTCACAACCATCCTTTCTAACAAAAAAAAAGTTGTTGGACGAAAGATTGTTGTCATCATATGTAAAAAGAAAATT
GCGTACCTATATGTATACAAACACATACACAAAATAAAAATTAATATTTAAAAAATGACTTGAAGAGACATTTTTTCCC
ACATGTTGCAAACTATCAGGAGGAATGTGAAGTGTGCTTAATGTCTTTAGCCAGTGAGAAAATGAAAATGGAAACTGCT
ATGAGATACTACCTGATACTCATTAAGATAGTCACTATTAAAAAAGTCAGAAATATACCAAACAGTGCTGAGAGGAAAG
GGAAATCTGTTACACTGTTGGTGGGAATGTAGCTTACTACAGCCATTATGGAAGACAGCATAAAGGTTTCTTAAGTTAA
ACAAAAAAAGAACGGGTGGTAATGAACCCTGAAATCCTAAGTACTCGGGAGGTGAAGGCAGAAGGGTCAGGAGCTCAAG
GGCATCATTGGCTGCACAGCAAGTTCGAGGCCAGTTTGGATAAAATGAGAACGTGTCTTAAAATGCACACACACACAAA
TACGGATGTTCGCATGCACCATTTGTGGGTTTAGGGACCCACAGAGGTCAGAAGAGGCATTGGATCCTCTGGAAATGGA
ATTAGAGAGATTTGTAAATATTTTATTTTAATGATGCATGTATGTGTAGGCGTGGGGGGTGTGTGTGTGAGTCTGTACA
TGTGAGGGTGCAGGCTGTGGGCTCTCATGTGAGTCCTGAGGTTTGTAAATGCTCTCAGATGCTGAGCCGCTTCTCTGGC
CCTCCTCGGGTCTTCAATACTTTTTTATAAAAAAAGTATAACTTAAATTTTTAAAAATATTTTGTTTTAATTATGCATG
TACGTATGGGCATGTGTGTCTGGATGACTTTGTGCCTGTGAGTGCAGTACCCAGAGAGTCCAAAGGAAGGTGTTGGATC
CCATGAGTTTGAATTTCAGGTGGTTGCAATAGTGTAGATGCCAGGATCTGAATCCCCCACACCCTCTACAAAAGTGGCA
TATGTTCATAAGTGCTGAGTCATCTCTAAATTCTAAGAGCGGCTTGCCTAGTATATGTGAGGCACAGAAAAAAAGTTTT
GAGGCAGGCAGTGGTGAAGCACGCCTTTAATCCCAGCACTCGGGAGGCAGAGGCAGATGGATTTCTGAGTTCGAGGCCA
GCCTGCTCTACAAAGTGAGTTCCAGGGCAGCCAGGGCTACACAGAGAAACCCTGTCTCGAAAAACCAAAAAAAAGTTGC
TGTGTCATCGTACTGTTGATGGGCCCTAGAAAGTCCTGTTATGGATGCTCCCTCTAGGTTTGTCTGTTTGTTCGTTGGC
TTTATTTAGTTTGTGATGCTGAGGAGCAAACAGAGCAAGTAATGTCACTGAGCTATTCCTGTAGCCCCTGCCTTTGATT
GGTGGAGTTTCTGATTTCTGTAATATTCTAAACTGTAGATCCACAGGTTGTAGTGAAGGGACAATATGAACCCCGTTTG
TCCTCATTTTGAGGACCAGGCCTGATTAAAAAAAAAAAAAAAAAAGCCATAAGGCACCAGCTTCGGGAAGAGACTCTAA
GTATCAGAAACTAGGCCATAAGACAGCAGCTCTAGGAACAGACCATAAAACCCAGAACAAGATCATAAAAGCCCCTCCC
AAAGAACAGCCTGGGATAACCACAAAACGGGGAAATATCTCAGAATGAGCGATCTGTGCTGGACAGCCCTATTTTATGG
TTGGACACTCATCCATAGGAATGTAGACCACTGACCACCTGTAACCTCTAGCTCACCTGTAACCCACCAGTGAAATCTT
ACATTACCTAATCCTTCCAGAGAGTTTCCTGCCCCCTCTAAGAAGGCCTGCATGCCTTTATCTAGGTTCGACATTTCAA
AGACTGGTTGACCTCTTCATGCTGGCTGCCTCTGCAGAATAAATGCTCTTTTGTTTTTGCATACTGAGTCTGGGGTCTT
CCTTCAGCGGTTTTCAGACCCTTACATAAGGAGGTGTAGATGGAGGATGCTGGAGACTTGGGTCTCAAGTATAAATATG
AGTACCCCAAAGTTGTTGGCATTGCTAGAAGGGAATCTGAAAAGAGAGGGGAACGCTGGGTCCAGAGGAGAATTCTTAA
GATACCCAGATTGGTAGGATAAGCTCAGGTCATGAGTGAACACAGAGAGATGGGAATCAAAGAGAATGACCATGTCAGA
GAGGGAAAACCATCCTAAGAAAGGGAGGGTAGTTGGGCGTGGTGGCGCAAGCCTTTAATCCCAGCACTTGGGAGGCAGA
GGCAGATGGATTTCTGAGTTCGAGGCCAGCCTGCTCTACAAAGTGAGTTCCAGGACAGCCAGGGCTACACAGCCTGTCT
CGAAAAACAAAACAAAACAAACAAATAAGAAAGGAAGGAAGAAAGAAAGAGAGAGAAAGAGAGAAAGAAAGAGAGAAAG
AGAAAGAGAGAGAGAAAGAGAGAGAGGAGAGAAAGAGAAAGAGAGAAAGAGAGAAAGAGAAAGAGAAGAGAGAAAAGAG
AAAAGAAAGAAAGAAAGAAAGAAAGAAAGAAAGAAAGAAAGAAAGAAAGAAAGAAAGAAAGAAAAGGAGGGTAGACTCC
CTCATGGAAAAGCTGGCGAGTAGCAGCAACTTCTGTCAGGGTGAGACTGTAGAGCCGATGACTAGTCTCTGTTTGTTCA
GCCAACTGCTTATTACTAATACATGGGGAGAAGGAGACGTAGAGCCTGAACCTTCCAGGAAAGTTGGCTGAGGAGTGGA
GGCATGGGGGTAGAAATTAGACAGGGAATCTGTCTAAAAGTCACTGTCAGTGTTAATGCTGAGCTCTTGAGCCATCCTT
CTATAAAGTACTCTTTTTAATGTTCATTTCGTACAAATTAGCCAAGGCCTTGTAGGGTGGAACTTTCTGATTTCCTAGC
ATGGGCCCTTACTGACTTAGCGGACTGCAAAGCTAGCCTGAATACCTGCCGCCTACAGGACTGTAACACTAGAGGGCGC
CATAGACATGCCCGCCCTCCGCAGCTCCTCCCCACCCTCACATCCTATACACCAAAGCTCCCAACACTTCTTCGACCAG
CACTCGAAGAGCTTCCCCTCGCCTGCCCATGACGTCTGCCCATGACGTCTAGCTGACTGGTGAGAAAGAACCTCGCCCA
CGGACTGACAACCAATTGTTTGCAATATTGTCGGGCTCGCAGGGAGATTTTTTTTTTTTTACGCAAAGCTGATTCAGAT
TTGGTTTTGGACCATGGCTGTTCCATCGCAATGAATTAATACAAGTTCTGTCAACTTTCTCAGTACTCACTAGGGCATC
TATTTGTAACAAATTCTTCTTTCTGGCATGTGCACATGTGCACATACGTACGTGTGTGGTCATACAGTTTTTTTGTTTT
TTGTTTTTGTTTGTGGGTTTTTTTGTTTTGTTTTGTTTTGTTTTTTGTTTGTATTTTCGAGACAGGGTTTCTCTGTGTA
GCCTTGGCTGTCCTGGAACTCACTCTGTAGACCGGGCTCGCTTCGAACTCAGAAATCCTCCTGCCTCCCAAGTGCTGGG
ATTAAAGGCGTGCGCCACCACGCCCGGCTAAGATTCTTATTTTTGATCTTTTATTTTATGTGTATGTGGTTGTGTGAGC
TTATGTGCCTCACATGAATGCAGGATCTTGCGGAGCAAAAGAGGCATCGGATCCCATGGAACTGGAGATACAGACAACT
GTGAGCCACCATGTGGTTGCTGGGAATTGAACTCAGGACCTCTGGGAGAGCAGCCTGTGCTCTTAACCTCTGAGCCATC
TCTCCAGCCCCAACTACCTGAATCTTGAGCTCAGGAGACAAGGTTCAGTTTATATGATATTTAGTCAGATTGTACAAAG
AAATCCTGGTGAAAATATCTAGAAAAGAATGGAGTTTGGGAAATATAAAGTCAGCAGTCGTTTTTATTTTGTGCTCTGT
GTATGTGGTAAGGCACAGCAGCGAAGACTACACCAGTTAGCCCCAACCCCTCAGGGCATGAAGACCAAGAGAAGCCCAT
GGGCCACGGGAGGGAGCATCTGTCTCCACCACAGTGGCAACCCACCTCTTGGATTTTAATGTTTGTGGAAAGTGCAGGC
TTCCAGGAGTCCTGGGGAGAGAAGAAAGATATAGGACATTGAGAATACTCTTACTTTTATGAGCTGTTTAGCAGAATGA
AAGGAACCTGAATGGGCAATTCAGTATCACATAAATTGTGCCAAGGCTCATTGTTCTGCTAGACTCATGCATGCTGTGC
CCCATCCCTCCCATCCCTGCTTCCCTACACAGCTCCTGAGCCTCCCACGGGGAGGGCTCCACACAAGGATAGAGAAGCT
GTCATGTGACTTTCCTGCCCTACAGGCATCCCCAACTTTGCTTTTAACCAGTGAGGCTCTGAACTTGAATTCACATAAG
GAGAAGCCGCCTAGGAAGGCCCTTCTTCCCTGAGAATGCGAGGCAGATGGTAGCTGGCAGTCCCCAGCAACGTCTGAGC
TGGGTCTAAGGATGGAGTGGAGAAAAACAAAGCTAGACTGGTGTTCTCTGGAGCATACAGGTAAGGGAAATATTAACTT
GGATTCTGTCACACTAAACCTCAACAATCACATTGCTCACACTAGAATTCCATGTGGCAACTTGTAAAACACATATATA
GCATAGCTTGGCACAGTGCAGCACAGTGTAGTATAGTATAGTATATTGTCTCCGACCGTCAGATCTAGGGATTAAACTC
AGGTCATCTAGCTTGTGCCACGGGCTCTTTTCCCCACTGAGCCATCTTACCAATCCACATGGGGCAACCTTGAAAGGGC
CATCACCCCAGAGCAGCACAGTCTCCGAGGCTGTATCTCCAGCACAATAACTTAAATGAAGGCCAAGACTCACCTCCAA
ACTCCAGTGGGAGACTCACCACTGTATATCCCATGCACCCCCTCGCACTTTACAAATGTTTTCAGGTGTCCCCGCTAAT
CCCCAAACAGATGGTAAAATATATTTTATCCTATTTCAATTCCTGGAATATGGCATTGCAGATGCTGCTTAGCAACTTC
CTAGCTAGCCAGAACTTATCATTATTGAAGTTCCCTAGAACAGAGCTGTCTAAAAGGATGTTCTATGAACAGAGCTGTC
TAAAAGGATGCTCTGTGATAATTCTTCTTGCTATGGCTACTTTCAATTAAAATTGTAGCTGGGTGGAGTGAGGATATGA
TATCTAGATTTAATCCAGCTGTCACCTGAACTTGCTCTGTCACACCTTGCTAGCCATTGCCATATTGGACAGGGACCTC
TAATAAGTTCAGTGCCCCTTTTTATCTTCTTAGGAATTGTGTGCATATAATTCGTCCACTGCCTTAGTATCCTGGGTCT
GCGACCCCTTAACAATATGTGCATATAACCCACGTGGTTGTATATGCTAGGTTTTGCCTTGCTCACGTGGCTGTTTCCT
AAGGGTTTGAGTTCTTGTAACTACCATTCCAGCACCCATCGCTGGGTTTCTCTGCGACCTCTCCACAGGGTTCATTCTA
CTTACGTGAGGTTGAGCACTTCCTCATGTTACCGACAGATTGCATGTACTCCGCTCCTAAGTATTTTTCAGGTGTGGTA
CCCTCTGGAAGTTTAACGAAACATTTGGTGTCATCCCTGAACAGAAGGTCCTTGGTGGTGGACTTGAACAAACAGAAAT
TGCCGGTGCAGTCACTTCCCCCAAATAAAGTCTGTCAAATGAGCAGAAAATAGACGTGAACCTATGTCTCGGACTGGAA
ACACCTGCAGGAAACTGAAAGGTCCAAGCCAGCCCTGGGAACAACAGAAAGCTACAGAAGATATTTATAATTGACATGG
CATTTTTTTAAAAATCGACACAAGAAACAGAGGTATAAAACGCTGTAGAAAGGTTAAACATTTCACATTGAGTTTCTTT
AAATATTTGGTAATGCTGCTATGAAACTGTTAGTGATTGTAAGACTGCTTGACTCAAATCTAATGTCAAACTAGAAACA
CCACTGAAGATTATCATAGGCTTTGGAAGGAACTTTTCAAAGTATGCTAGCCTATTTCTTTTAAAACTAATGACAAATT
AAAAGGCAGGCAGCTATACCAGAAGGAAGGGTAATTTGTGTGTGTGTGTGTGTGTGTGTGTGTGTAGAGTGGCCCTAGG
GATAAAGTGTTCCAGTGGCCCCTGTACTCGCTGCCCCCCAAGGCTGGCCCTGACAGGATTTTTCTGAGAGTAAGAGTCT
TGTATTTTCCAGACTGTTAAGAATATGGTGCAACTGATACAAGCAAGAGAAGGAGTAGTCAATTCCGGTCCTTAAGACT
CATACGAAGGAACATTAGAAATTGTTTTCAGAAAAACATTCTCGAGGAAAATAAAGTCCCGAGTCAAGATCCAGATGAG
CTGGAAGGCAGATTCTAGGCTCGGAGGGGTTATGGGTCAAGTGGGAATTTGTCTGTCCTGACTAGGAGTTCCACAGGAG
CAGAGCCAGGTCTGAGGAATCCAGTCCCAGCTAGCACACTGTGTGCCCTCCCTGTAAACACACATTCCCTAAGACGTGT
GCATGCACCACTTTATTTATTTATTTTTTTGTTAACTTAGAAACATGTGTGTATGTGGTATTATTTCCATTTAATACGT
GAAGAAACCGAACGTGGACATTTTAAGCCATGTGCCTAAGATGGCGTGGACCGAAAGCTGCCGAGCTGAGATTTGAACT
CAGATATTCTGATCCACGGTCTGTAGAGTCCAAATTCTAGTTTTTAGAAAGGAAATGCTCACTCCGGAGCTCCGTTTAA
CTCCGAATAGCCTGCCCCTGCTAGGAATAAACGGTAGCAGACCCTGGGTAGTTCATACCTCCTGGCTAGTCAGTACAGC
CTTAACCCGGGCTGCCTTCTCTTTTCGTGAGACCACAACATGGTTTGGAGCTTGGGCCAGGTGGCAGCTGGCAAAATCT
TTCACAGGCTTCCTGGTGCCATCAGGGCAGAGCAACTCGAAGTCTTCCTGCTTCAGATTCTTAGCCCATTCGGCAGGGT
TCTTTCCTGGGTAGAGAAAAGGGGTGGATCCGCCAGGTGGAACCGTTGTAGTCACTGGGATGTCACTGACCCACAGGTG
TATGGAAGGCGTGTGCCTTCTGTCCAGCACAAGCCCATGTTTAAGGTTATGGTGGTGTTAGGAGGAGAGGATGGTCGTG
TTCATTACAGGTAGCACTCGATTCACAAGAGCTTTTTAGTGACCACTCTCCTCTGACCTGCCCTCTCAGCCAAATGAAA
AAAGAGCCACAAGCCAGGCAGTGGTGGTGCTCGCCTCTAATACCAGGACTTGGGAGGCAGAGGCAGGTGGATTTCTGAG
TTCGAGGGGCCAGCCTGGTCTACAGAGTGAGTTCCAGGACAGCCAGGGCTACACAGAGAAACCCTGTCTTGAAAACAAC
AACAACAGCAACAATAACAAAACAAAACAAAAAGAAAAGAAAAGAAAACAAAGAAAGAAAAAAAAAAAGCCATGTTCTA
TGGGTGGTTTTTTTTTAAAGAAAAAGAAAAAAAACTTCTATTAAATTCCAATGCTAAACAACAACAATGCATTTATTTA
AATAAATAAATAAATAAATAAAGCAAGCAAGCAAGCAAGCAAGCCAGGTGTGCTAATGCATGCCTTTAATCTTAGCACT
TGGAAGGCAGAAACAAATGGATCTCTGTGAATGTGGGGCCAGCTGGTCTACACAGTGAATTCTAGGTTAACGGGAGATA
CAGAGTGAAGCCTGTCTCAGACAAAATCCCGAGACCTTCTGTACTTCTAGCTCCATCAAGGAGCGGGGAGAAGGTGGTG
CTCTAAGGCAGTTTCCCCGCCTCTGTGCTGCCCTCTCAGCTCTCCAAACGGAAGACCCGCCCGGAAGACGCTCTCATAT
CGATACTCATCGATATTTTCAGTTTGACTTCATGCTAGTCCCAGGAGCTAATGTCCTATCTCAGCTGCTTACTTAATCT
TTTCTGCATTCTCTTTGCAACAGTTAACCAACCGAACAGTCGATTTAAAGACCTCCGCCCTTTCGGAGGTTTTCCGCGG
TTCCTCCGGGCATTTTTTATTGCCAGTTTTCATTTCTCGCTCATAAAGCGGGATCGTGACCTCTCTCGATTCATTCACT
GTCCGCTCAGCTTTTCCCGTAATTGAACAAATACTGTGTGAGACCAGCGTTATCATATTTTAAAATTATTCCCTCACAA
ACTAAACACCCTCACTTCTGCGTTCCTTTACTCCCAATGTTTTCTCTGGCACCAAGCCTGGAAATCACAAGTTGTGCTT
CCACGCATGCGCCTTGTCCTCATTTACCTATATACAAGTCTGTGTGATGTTACAAGCCTACATTTTTTTTCTTAGCAGT
AGATCTTAACACATATGAACTTCTGTAGCTCTCAGCTACTTATATGCGTCACAGTTTCTTCTGCACCATTATGCAAATC
AAGGTAATTCTTTAAAATCATTGGATCTTATTCTATAGTGTGACAATAACACAGTGTTTTTAAACCATTCCTCCATTAA
TAGATATTTGGAGTAATTATACTTTTTTTTTTTTTGCCACTACGGTAATGATGAGTGTCTATTGAATATGAATGTGCTT
ATGTAGATGTAGAACAGTTAATTCAGCTTAATTTTAATATAATCAAATTGCTTCCACCACCCTACGTGGTAAAGACTTG
ATTAAGCATGATCCAGGCCTTGGGTTAATTTCCAAGTACTAAGGTGGGCTGATGGAGGGACAATGGGAGGGACTGGCAG
AGGATTATAACAAGGTTCTCTAGAGACAGAAGCTATGGAAAGAGAACTAGGAAGGGCTTGCATTTGTGCCAACTGAGCT
TGTCGTGCCAGCATCCTGTGCATAAAAATAGCAGCTTGTCAGTCTGAGGAGCTGAGGAGATGGAAGGTCAGGTCTCCTA
CAGCAACTGTAACCAGGAGGTTACAGGTGTTGGAGCCTTTATGAAGAGTGGAGAGTCAAGGCCTAGAAATCAAAGACTT
ACTCAGAAAAGCACAGAAGAAATCAAAGGTGACAAAGAACCAAGACCAGGATGACACTTGGGTGGCATGAAAGAAAGCC
TTTAAAGAAAGTTTAAAAATCATGTGGGGAACAAGGGAGGGAGAGAAGGGAGAGAGAAAGGGGGGACTGAGAGAGAGGG
GAGAGGGAGGAAGAGAGAGAACAAGGTAGGAAGAGGGGAGAGAGGGAGGAAGGGATAGAGAGAGGAAAGGAGAAGGAGA
GGAGGAGGGAGAGAGAGGTAGGGAGAGAGAAAGAGATAAGCAGAGGGGAGAGAAAGAGAGAGGGAGGGAAAGGGGAAAG
AGGAAGGGAGAGAGAGGTGGGAAGGGGAGAGAGAAAATGTGTGTGTGTGTGTGTAAGAGAGAGAGAGAGAGAGAGAGAG
AGAGAGAGAACACAATCAAATCAGTTCGTTAAATAAATTCTTAGTGTCTTCTGAGTGCAGTCTCAGGTGCTGGGAGGAT
AAAGAGGAAGAGAAAGAGCCCAGTGATTGCCCACAGCCAAAGTGCACACAGATACTTTGTAGCCACATAGACAGAAGAT
TCTCAGCAGAGTGGGTGTGCCGCCTGGGAAGGCGTCTCAGAAGAGCTGGTGCCGAGAGACAGTCCATCCTACATCAGTT
GCCCAAGGCAGTGAAGAGAACAGACCTGAGAGCTGACAGGGCAGTGGTTAATGGCCGGAACGGCAGTTCCTCAGGAGCC
CTTGGTGACATACAAGGCCTGGCGTGCTCATCCTTCCTGATACTCAAGCCCATCTTCCCAACCTTGCCATCCCACCTCA
TTCCTTAGCTAGCATTCCATGGCCCGCAAGAGCTATCTCTTCATCGTTTATCTAGTCTGGGTTGCATCATGGATTCTTA
CTTTAATCTATGGTTTGCCATCTGTTACTGTTAACATTAGTTTCATGTCCAACATTTCCAGACATGGCACTGGGAGATG
CTATACACTGGCTCCCTGTGCTGTATCCCATCCTCCTTTGCACACGGCAGTGCAAGCAGCTTCCCACGCATATTCACCC
TGCCCCAGCCTTGCAGGGACCCTTGCCATAGGGATCACTGCAGAGCAATTGCTTCTCTCTCCATTGAACATACAAGTTG
AGAATTAGAACTCTCGAGCCTGACGGTTTCTGCACAGCTCCTATGGAGCCATCCCTGAGTCTCAGCAAATCCCAGTGAC
CCAGATTCTGGAGGGAACCAACAGAAGAGAGCGCACACATACCTTCGGTGTTATCCAGGACAGTCTGGTGTTTCACAAA
GGCTACATCTCCTTTCTCAACGAGACACCTGAACAGGTCCAGATTTGGAGAAGAGAGAAGACAGGAGGTCAGACTGGCA
CCTGTCATTCAGAGGCACCTTGGGGATTCAGGTTGGTCACCTGATGTCATCAAGTGTTAGGGCCACTCGTTCTATCACT
CAACAACATGCAGAATAAGGAAAGCAAAAGGCCCCAAGTTGCTGTGCAGTCAGTCCCACAAGCTCAGCTCCACACACCA
TGCTTTTTTTTTTCTTTATTCTTTGTGGGTGCGAGAGGAAGGCGTATATGCACGTTCATGTGAGTATGTGTGCATGTGC
ACATGTGTACATGTGTGCAGAGGTCAGAAGTCAACATTGGACATCTGCCTTTATCATTCCCCACCTTACTGTTTGAGAC
ATACATGGTCTCTCCCTGAGCCTGGAGCGCCTCAGTTTGGCTAAGCTGGTGGCTAGTGAGCTCCAGAGACCCACTTGTT
TCTCCTCCATCCCACACTCGCAAGTGCTGAGGTTAAAGACATATATTCGTGCTTGGTTTTTATGTGGATTTTAGATATC
CAACTCTGTGTGTGTGTGTTGGAAAACTCACCTGAAAGCCCCTGTGTAACCATTATATTCCTCTTTGTTGTTCGGAGCA
CATTTGAGTGGGCCAATACACAGGTCACAGAGGGTGGAATTCTTCTCATACCCGGGAGCGCAGCCTTGACTGAAAAATT
CATCTGGACAGAAGAACAAGAAAATGTCAGCTTTGTGTGTGTGTGTGTGTGTGTGTGTGTGTGTGTGTGTGTTAATTCT
AGTGGCTTACCTAACCCCTGACAGGCTATGAAATAATTCACTCTGCAACCTGCCACCCACCAACCCAGAACCACCCAGA
CTATAGGTCTTTGGAGGAAACTTCTCAAGGTTGGGAAAGGATTTTGTGAGCATTCCACTTCAGGGTAAAGGAGACCCCT
GGAGGACTGTGATGGTACCATCTCTAGCCTGTAGAATGAAGAAATGGAATTTCTAAGGTTGTACCATTCTTTCTTTCTT
TGTGAGTTTCCCCTTCCCTGCCATTCATTAGAAGAGCTACACTACACCTCAGAAATCAAGAGAGGGATAGGAGACAGCC
TCTGCCCATCAGCTGCTTCTATGGATGTGAAAATATAAGGAGGCTCAGGGAAGCAAGTTCAGGAGCCTGTGCTTTCTGC
CTGTCACCCCAGGTGTCCTTTGTTTCTGAGACAGCAGAGCATCTCTGTGCAGAAATGTGTCCCCATCTCCCCTCCCTGC
ATATTCTAAGGTCGCAGAGTTCATAGTCATGTTTACAGTTCTATCATTTTTAAGGAGTTGCCCAAATTTAGAGGCATCA
GGAAAGAAGATCTGAGGAAGCCAATATGAAGCAAAGCCATGGCCAAAATTTTCTGTTGGGATCTTCTACTCAGAAACAT
AAGGAGATAGAAACAGCCTCAGGTAGAGGACATAGTCAACTGAAGAATCCTCAGGGCTGAGCAGGGACTCGGTGAGATG
ACCAACACCTTAAGGGACCCAGGAAACGGCAGCCCACATAGTCATATCACAGAACCCTGATGGCAGAGATTGCTTGATA
TCCACCCAATGTCTGTTTCCCTTTGTTCCTCCGTAACAAAAATCTAATTTGGTTTATAGTAGGTACATGTAGGTAGGTG
TCTGACAGGACACCTAAGTTCTGCACAATGGCAAGTTATATGCAAATAATTGCTTTCAAGTCCAGAAGGCTCTTTTCAA
GCACTGACCATAGTGGGTGTAAATTTCCACTTGTTCTCCTTGCCTGAATTGTGACTGCCCAGGCAAGGTTGCTTTGAAC
ATGGAAGTACTATGATGGAAATCGTGGACCGGAAAGAAGGAGAAGCTCTCAAGACCTTATCAGCCGTGGACTGCTCACC
TCTGATCTCCATCATGGGAGAGGCAACTAAATGTATATCTACTGACGCCATCATTTGCCACTGAGACAATCATCCTGAC
TCCATGGCTCAGCTCCGCACCCCTCCTAGCTTTTTTTGCTATTTCCTTGAGGTTCATTTTTGTGAAACTCACTTTTATG
AACCCTGAGCCCACCGCCTCCCTCAGGACCCTGTTTTTCTGCACGGCTCCTCCCAAGCTGCCTTCCCAGCTCTGCTCAC
CGAATTTGCAGTGGTTGATCCTGTTGTACAGCATGCCCATAGGGATGTTCCAACCAGCGGTTCTGTCTACCCCAGTGTG
GCAGGACTTCTTGCCTTTCAGGTTGTTCCAGGTGATGCTAGTGTCCGATGCCTTCACCACAGCCACGGCATAATACCCT
TTAGGAAAGATACCTGAAAAGAACAATTAATATCGCTGGGGCCTCCCAAAGTATCTGCTTCCAACAGAAGCCACTGTCT
CCCTAGGACAGCGAGGATGTCCTAAATAGCATTGTGTCGTGAAAGGAATTCCACAACATCTAACTCCGGTGAAGAACAG
GACAGTCTTGCAGGAAGAATCTTAAAAGCAAAGTACTAAGATCACATGGTGCCCCGTGATTATGTCCATATTAAAAATG
CTTTGATGTATTGATTTAAAAATAAAATAATGGGCTCAGGAAATGACTTGGTTGGTAAAGTTCTTGCCATGTAAGCAGT
TCGGTCCCCAGAACTGATGTAAGATAAAAGCCAGGCATGGTGGCATGTGCTTATAATCTCATTACTAGGAAGATGGGTA
CAGACAAATTCCTGGGCCTTACTAGTAAGCCAGCTTAACTTACTCCAGACCAACAAAGATCCCATCTCATAAAACAAAG
TGGATGACTTCTGAGAAATTCCACTTGACGTTGACTTCTGACCTCTACATGCCCCATCACAAGTGTGCACATGTGCCCA
CATATACATGTATACACATAAAATAGCAAATATTAACATATAATCATTAGGAAGAAGGCTAACATACAGCCCTTACCTG
TATGCATAAGGTTATTGAAGTTAAAAGCCAGTAACTCACTTTCCAAATTATTAAATCAAAGACATGGGTACAGAAAAGT
TAATGGAACTCTGGCATTTTGTATATGAACATGTATTGGGGTTAATCAGGCCAGATTCCCTCTGATAATAAGAATATGA
TTTTATACCAGGATAAACATAAAAAAGGAAATACCAACAACACATTGGTTGTAATTTTCTATAGCACAAATGTGACCTC
AGTTGGGGCTGGAACACCTGTGATTCTGTTTTCTGTTTTAATTAATTCAATATACTTTCTCCAATTGAAAACAATCTGT
GGAATAATAATGGCTCCTTCTAGATATGCTAAAAACTGGGAGGTTAAGTTTAGCTGCAGGGACAAGACTACAGAATTGT
ACACTCATAGTTCTGTGGAATACAACCTAAGGAGACTGAACACTAGGACTCAGGGTCACTTTGGCATTTGAATTCCTGT
TTGCTCTGGTGCCATCTTTTCAGATCCACTCCTAATCAAAGCCTTAGTAAGAGATGGGCTAGTCTGGTTTCCTGGGCCC
AGCCTGGATGGAATTCCATGCACTTAGACATCAGCATTTTGAAGTGCTCCATTCAGTCCTGCAAGGACTACATAGACAA
GGTCTGTGCTAACTCTCATCTAAAACTCTGAAGACTTCATAGGATTCAAAATGGTAGGAAGCAATGGGAAGAACCTAGA
TTGTCCCCAGGATCAGTTGACAGGGGCAAGAAATCCTAGCTTTGTCAACAGATGTTCTAGTTACTGCTTTGTTCAAAGA
TTTGTTTGGAGACCATTCATAAATTCTCAACCCTTGGGTTCCCTCATGCATCCTTATAGAGACAATACCTACATATTAA
CAGGAGATGAGTAACCCTGTATCTGAGTCACCACACACACACACACACACACACACACACACTCCTGATCAATTACTAT
TCTTTCATCTCACTTTGCCTTCTAAAATTGGCTTTCTCCATTGACTTATAAATTTGCCTAACCACTTCTCCCAACAATG
AGAATTTACCTTCCTGTGCCCGAGATTACCCGTTTCAACTCACCTTGTTGTGATGGGATGGCACAATTAGAGCTCTCTG
AAAGAGAATCAAGAAACCATTAAACATTTATGTATAGGATAGAAAACCCACACCTTTCTCATGAGGCAACTAAAACTAA
GAGGCATGGATAAGACCACACACACCTCTGATTTACAATAATGTCAAGACTCTCCAGATGTTGAGAGTTCACCTGGCTG
TTGACCTCTGGGACTCATTCACAAACTGCATTTCAAGTGTCTTGCTCACTGTGTACCAAGCCCACAGTGGGATGTTGAC
TGAACTTATTTGTCCTGCTACAAGGTAGCAGGAAAAGTGAGCCTTCTGGATATCGCAAGACTTTCCAGACTTCCTATAT
GTCCTCTGTAATTGGATCCTTTTTATACCAGAAATCTCATCTCAGGGACTGAGGATCCCACCACTCTCATCCTTTAAGA
TGACTGTGGAGATTCTAGGTGAGGGCGTCTCAGAGACTGAGAAAAAGCTCATGCAAGGAGCATTTTATGGTGGCTGTGG
GATAAATTGGGCTGTGATTCTCCTTCTACCATGGCTCAGAAAGAACACAGAGGCACTGTCCTCACTCACCGTAGTACTC
TGCCATGACAGGCACTAGACCACACTGGCCTGCAATGTAGGCATGTCCTCCATCCAAAGTCATGGCGTCCGCTTCTCCG
TTCTGCAAAGACACAGGAAGACACAGCAAATGAAGAGTTGCTATTTCCTGTGCCACCTGCCCTTTTAGACTGGCCTCTT
GAAGGCCCCTCTCAGCCCAAGTTATAAAGCCAAGGGCAACAGTAGATGTTGGTACCGGACCCCTGATGATCAGTCAGGC
ACGCCCAGATGTTAGTGTATTTTTATATAAACTCTGGTTCTGTGTTAGCAAGCTTTGTATGAAGTTGGTTTGATTTTTC
TTGCCTTGACTCAGACAAAACAAGAAAATGGTCTGTAGCTAAAAGAATGTAGTATCTGAAAATGTAGATAGAAGAATGG
GGTTCGCAGTCAGTTTTGCTTTCTGATAGATATCTTAGAAAGCACAAAAGAGTCAACTCAAAGAATAGGCAACTGGAGA
GGCTCCATAGAGCACAGAACAGAGGCAGGGGTGTGAGAGCTTGGAGCTTCCTTGCCCTCTCCAAGGCTGCCACCCTGCA
GAACCTCAGTGTATTCTCTGAGGCAGAAACGCCAGGGTTCGAAAGACACAAGAAAGGCTCACAAGACACGATGAGGTGC
TAAGGTGGGGTGTGCTAAGCAACCCACACAGCACACTCTCAAGAAGAGGGTTTTCTCTGAGTCACGGTGATGACAAGGG
GTGCTGTTTTTCTTCGTTCTCGGGCTCAGGATTTAGAAAATTCTCTACAGGCAATTTAAAAATGCTATCTCGATCAATG
AAGTGAACATGTACTTGCTTGCGAAATCAAAACGGAGCCCTTTCCACACTCCCCAGGGCACATCCTGAGCCCAAAAGGC
ACTCTTGTCATGGTCCAGTGTCCTTTGACAGGAGATGGCTCAGTTTACCCTTCTACTGCGCTAATGTAAAGGGCATCCT
TCAGAGCTGAACTCAAACCATGAGAGCAAGGAGTCTGGAAAGGGGATTTCCATGCAAGAGTTACACTTGCTAACTCAGC
GGGCCAGCAGGCCATAGTGTTGGGGACCCACCCTGAGAGGCAGGAGACACGTACCACAATCTTTTCAATGCAGTCCTCA
GTGGTCTCTGCTGACTCACACTCTATCTTTCCCTCACTGATGATGCTCCACTCGTCACACTTGGTTCTCTCCAGGTGAC
TCAGTGCACACCACTTCACTGGCGAGTTGTCGATCGAGCCCTCCGGGCCTGTGGTACCAGAAGAAGAGCCGGGGCTCAG
GTAAGAAAGGCTGCAGCAGCTGTCTGGTAGATGTCAATGCTGCTAAGTGCTTTATGGAAATGATGAGCTAAATAAACAG
ATCCCACCAAGGAACTCCTAATCGTCTAGGGAAGCAGATGGGACACAGCCCAACTGTAGTTGATTTCATTTAAGTTCAG
CTTTTCTTCCGAGGTAAGATTTAGTTTTCTTTAATTTTAATCACGTGGGGTGCGTGTGTGCGTATGTGCGTGCATGCAT
GCGTAAGTGCATGAATGTGTGTGTGTGTGTGTGTATGTATGTTTGTACATGTTAGTACAAGTACCCTCAGAGGTTTGAC
TTTGGTTTTCACTACTGTAGGACTTTGATTTTTTTTTTGTTGCTGTTTTTCATTCTTTCATTTTGTATTTATCTGTTCA
GTAGTGTGACTATCTCTCAAACATTTGTCCTCTCCCTGACCTGAAATTCCTTCCCCAAACAGCAGAGTCTAGTATGTGT
TTATAAGTTAGGCACCCAGCCGAGTCTGTGGCTGGTTGTCTAATACATACCTACTGAGTGAATAAATTATTAGGCAAAC
CCCCAGTCCTCCAGTGAAACCCAGGGCCAGATCAAACTCCCTGCATCTCCAGCTTTCATTACGCCCATGTTGACCACAA
TATTGGCAAGAGATATACAGTCTGCAGAAACATGGAGTTATTTACTTTCTAATAGTCTAATTGGCTTCATAAAGGGCAG
GGGGCTACCCTAATTTTTGCAGAGGAATGATACTAGTATGAACCCAGGTCATTTTAAATGTGACCAGCCTTTAAAGCAC
TTGGTGACCTCTGGGTCCTCTCATGGTTGATGCCAGCCAGCATCAGTCTAGCCCTAGGCTCACAAACAGGATGGAAATG
AGAACAGCAGGAGAAGCTGTCAGTCCCCAGAAAGACCATGGCTGCTTCTTGACCTTACCAGGTTCCCCACATTCCTGGG
GTCACCTAGTTTTCCCTGAATACTTACACACGCCTTCCTGCTGATTCCGAATGGCAGTGACATAGTTATGGCCAAGGTA
CAGCCTGTAGTCCATCCTTGGGGGGACCCTTAACAGCCCAAAGGCAGAATCTTTAAACAGCAGGTCTTTCCCAAGAGGA
GAGCTGAACAGTTGGAAGTCTTTTGATTTGCCTTTGCCAAAGTGTTCCTGTTTCCAGAAGGAAAAACAACAACAACAAA
AACAAAACAAAACAAAACAAAAAAACTATAAAAACAGTGGAGTGAACAGGGAATTAGGGAGGTTAAAAAGAAGAACAAG
AACTGAAGAGAACAAGAGAAACTTCAGGAAACTGATCTTAAAAGTGTGGGAAACTTGGAGGTACCAGGAGAGAAGTTAT
TCGGGCATGTTCTCCATCACCCAGCGCCTCTTTGTCCGGGATCCTGTAAGGACCATCCCACATTCCCTCTCCCTGTCTG
CCTGAGTAGTTAAGTGAGAACTAGCTGAGAACAAGTGAATCTAGTGCAGAGGCTGGCATGAGTAGGCTTGACCAGAGGA
AACTGTGTTAACCGACCTCAGCCAAGAGGCAGGGGAAGTCAGTTCCTCTGCAGTCTGGGTGTGTGCGCGTGTGCCTCTG
TGTGTGTGTGTGTGTGTGTGTGTGTGCGTGCACGCGCGCGTGAGCGAGTGTGACGGAGATTCTCTTTCCTTCTGGGATT
CTCTCTGGGCCTTGAGGGCCTAATTCAGATGGAAAGGGTATCATTGGTCAAAAAGGTGGTGGATGTTGATACCAGAGGA
GACACTGGCTACTCTATGAGGATGCTGGAGAGGTTGGTGACCCGGCTGTCTTAGTGTGTCCAGATGTCAAAGAGATCTC
TGCACAGAGCGGTAAGCAGGACAGAGGATGGCGGTAAAATACCTGTGCCACTTTGAGAATCTCCCAGATCAAGTCTTCC
TTGCCATTGTTTTTTCGAGCCACAACAGCATGAGAAGGGATCCGAGCCAGGTAGCAATCCTCATACTGATCCACTGGCT
TGCGGGTATTGTCAAGGCAGAGCAGTTCATATTGGTCCCTGTCAGCCTTCTCCGGCAAGACCTCTAAGGAGGGAAAGTT
CAGTGAGCCTGTGGCAGGCTGAACCCCTCCCCATTTTTGAAGTCCTGTTAGCATTGAAAGCATGAACTGAAAGGTCTAG
AGGGATCTCTGAGGGTCAAGGAATATGCAGGAAGGTGAGCCTATGACCAGTTAAAGACACAGAGGCTTCTGTAGGCCAG
TGAAGCAATGGTTGCCCCCATCCCCTGACTCCAAGCTCAGCAAGACTACTGACAGGCAAGGTCAGCCAGGCCAACCTCC
AACCCTTGGCTGTGACACTTCTTCCCTAGAGGGAGAGCCATTGTGTGGTTTGAGTGTTGGAAAGAGACCCATGAATCTT
GGTAGAGACCCCCAAAGTACCTTCCTCTGCATATATAGAGACTCAAGCAGCGGGAGAGAGACCCTGCATGCTCAGGTCT
GGCCACTAGCCATGAAAAGGATGACAAGGAACCGGCCAGAGTCTTTCCCCATCACTCACCAAATATGGTTGTGTGCTTG
ACAAAGGCCACATCCCCACCGCCATCTTTCAGACACCTGTGGGAAAGAAGCCCATGAACAAGGAGTCCCAGAAGGTGGA
CCCTGCTCCTGGTCCAGATCACACTGTCACCTTTCAGACAAGGCCTGAGGCTGAGTGGCCCTCATAGTGGAGACAGCTC
TCTAGGGTGGGATGCTGACATCCACAGTACCACTGGGATGGGGGATGTGCTCACGTACCCCAAAGTGGCTGACAGGAGG
TGAAAATAATGTAAAAATCTTCAGATCCTTAGTATGGAAGATGACAGTATTTCTACCCTGGCTATGTGGATTTCAGGGT
ATCAGAAGACAAAAAAGAGAGAGCAAGAAATTGGGCATAAATGACACAAAATCCTATTCCTAAGAGGCTACTTGCAATT
GATGCTGGCTTCAGAAAATCAGGGTTGTTTGTTTGTTTGTTTGTTTCAAGATAGTGTTTCTCTGTGTAGCCTAGCTGTC
CTGGAACTCACTTTGTAGACCAGGCTGGCCTTGAACTCAGAAATCTGCCTGCCTCTGCCTCCCAAGTGCTAGGATTAAA
GGCGTGTGCCACCACTGCCTGGCGAAAATCAGTTGTTTTTGTTTTGTTTTGTTTTGTTTTTGTTTTTTGTTTTTTTAAG
GGAGTATATCAACCATATTTCAGGACCAGTCTCATGCCCAGGAGTAGTTGGCCAACACACACACACACAGAGGACTTCA
TGTTTTGTTTTGTTTTCCCCTGTGTACTTTTGGTTTGGTTTAATCTTCAGTGTTGGTGTGTGTGTGTAAGAAGGTATGG
GGGTGCAGTGTCCTCTGGTATGGTGATAGAGACCCAGGGGTAACTTAGGCAGAGCTGGGTGGTAAGAAGCCAGAGAAAC
CTCTGTAGACTTAAAGACAGATGGTCCATCTGTAGCATCATTCCAGGAGAACAAAGGCCCAAGAGGGACCGGCACAGCG
CCTCATTGTCCTTACCAACCCAAGGCTCATGGAGCCATAATAGAGGCTCACATGCCCTGCCTCCCTCAGAGTATTTTAT
GTCCACTGGACAGTTTTATATATGATTAAAATATATTATATGTAAAAACATAATTTAAAAAGCCACATGTACTGCTGGG
GTGGTGCGGGGCAGCGTCTAGTATCCTGAGCATGGTCTACTGTGAGGCACTCGGGGTATGTAGGTCCCTTCAGATGCTA
CCCAGCCCACTGAGAGGAGACGCCAGCCAGATGCGGAAGCTCTGAGGAGAGAGGACTGCCAGAACAGGGCTTGAACTTT
GACATCAGATGGAAGGGGACATAGGTTACCTTGGCCTTGAGAGCCCCAGGTTGTAACCCCGCCTTCCTGGGAGGACCTT
AAGCCACCTGCATGCTTTTAGGTAGGTTGGCTCAGCAAAGAGCCCCCATTTGAGGACTTTTGGCACTGGCCACCACAGA
GTGTCAATTCCACTCACTTGAATGCGCCTACGTAGCCAAAGAATGGTTGAGTGGAGGAGCAGCCACAGCCTGGGCACAG
TTGACACAGTTTGGGGAAGGCCACTGGATCTGCACAGGGGACACAACTGCCCGAGAAGAAACTGGACACAGCTGCTCGA
TACAAAGAAATGGACAAGGCATAATAAGCATAATGCCATCACCCAGCCTGGGCTGGTGAAGGAAGGCAGAGGCACAGTC
CCTGCCCAGCACAGTCCCTGCCCAGCCTTTCATGTACTAAGCAGGGTTTCTAGACAGGAATAAGTTAACCCCTGCTCTT
CAGTTACCCTCCCGGGGACACAAGCAGTACTTCTTGCTAAACAAGGCTTGCCCTAGACTTTGCCCACTCTGTTTCCAGG
TTTTTTTTTGTTTTTGTTTTTTTCAAGACAGGGTTTCTCTGTGTAGCCCTGGCTGTCCAGGAACTCACTCCGTAGACCA
GGCTGGCCTCGAACTCAGAAAGCCACCTGCCTCTGCCTCCCAAGTGCTGGGATTAAAGGTGTGAGCCACCACTGCCAGG
CTCCAGATCTTTCAAAGGGTGCCTCTGGGGGCTGTAGTACAGCCTAGAGAGGTGCAGGACAACCTTATGACCAGCAGGT
GGAACCAAAGCATCCTTTAACGGAGCAGAGGTGTGTGTGGTGGTGGGGGTGATGGGGGTTGAGGGTGTGTGAGGGACTG
AAGTGAATATGGAAATCCACTGTGGACCCACCTGCCCTTTCATTCCTCCTCAGGTCAGCCTCTGCAGGGGTGTTGGGAG
CTCACAACAACAAGAAGGTTCAAGTTGCCACTTCTAATTGTTTCCCTCTTCCTCCACTTCCTTGGAGTTCAGCCTACCT
GCCTGACTAACCAGGACTGTGTGGCCCCAGCAGTACAGGATATTCAGAATAGAAATGGCGGCTTACCTTTCTCAAGAGG
ACTGCGGGGCTCCGACAGCTTACAGAAGAGCAAGCCAATGGGGATGACCCAGCCTGCAGACCTTCCCAGGCCTGTGTGG
CAGGACTTCTTGCCTTCGAGCTGGTTCAGCTGGAAGTCTGTTCCCTTCTTTACCACAGCCACAGCGTAGTAGTAGGTCT
GTGGATCTGAAAGGGGACAATGTGGAGGGAGGACTTTGACATGGCCCAGAGAAGCACTGCTTGTCAGTGCTCTGTCCTG
ATGAAGGAGAGAGAGAGACCCAGGGGTAACTTAGGCAGAGCTGGGTGGTAAGAAGCCAGAGAAACCTCTGTAGACTTAA
AGACAGATGGTCCATCCGGGAAAACAAAGGCTCAAGAGGCATCGGCACATGGGTCATGAGCGCCTCATTCTCCTTACCA
AGCCAAGGCACCAGGAACATGTGAGAGCCACAACAGAGGCATTCTCTGTCTCCCTCAAAGTATTTTACATCCTCTAATA
TGGTGACAGGGACCACCAGAGCTATCTCAGAAGAGCTTCTTGGACTGCTAAGCTCCCTCCGGGCACTATAGGGGTGGGT
TTTCTCTTGTCCATGGCGCCTGAGGCTCTGGGGAGCTTGGACACTTCCCCAGATCCAGTGAAGGCCAAGCTGGACCCCA
GCCTGCTCTCCATTGAGGATGCCTGACTCAGAAGGTCCATACCAGCCTCCAACCAGCCCAAAGTCCTCAAGATGTCTCC
ATGTGTGCCCGGAGGGCTAGAAGATCTCATTCCCCATGGACTCCCCAAAGAAAAGGCTTCCTAGCGTTTCTAATGTCGG
CTACCTGCCTACAGAATGAGATTTAACATCTACTAAACCACGGTCCCCCTCCTGGGTATTCTGGGAGAACTCACGTTCC
ACTGATCCATAAAACTCCGCCGCCACGGGCTTCAGGTTGTTCGGAGTCAGGCCGGCATCGTACACCCAACCCCCATCCA
AGGTCATAGCATCGGCTTCACTTGCCTAGAAGAGAGCGAGTCCCAAAGACTTATTGCAAGGAAGGCTTCCCTACACCAG
AACAAGTGGGAAGGACCTGGCAGCGCTCCATGGGTCTCGGGCTAGAAAATCCCAAGCACAAGCACATCCAGCCCTGCCG
GTTTGGGTTGATTCCATCTAGAGGAAGAAATGGAGTCTTTTTTTTCCTCACTTTTCCCTGATGATGTGCCCAGTACCTG
GACAAAATGCGGATTTACCCATCGGCAGTGGTCGAGGCTTGGTCTGGCAGGCTTGGTAGGGGGGAAGGGTCTCTCCCTC
TTCAGAGGTGGGCACTGCTATTATACCAGAGGCTCTGGCATGGTTGTAGTGGAGCACCCAGGGTCTGTGGGTGGCTGGG
TTCTTATATCTGTTTCCATGCTCGGCCACACAGTGGGGTTTGTCATTCAGAGACCCTCGTGTTTTGAGAGTCCCTGTAA
GCTCCTCTGTGGGTGCTGTCATGGAGGTGAAAACCCTGAAGGTGCTGTCTTTGTTTTTTTTTTTTTTTAAAGATTTATT
TATTATTATATCTAAGCACACTGTAGCTGTCTTCAGATGCACCAGAAAAGGGTGTCAGATCTCATTACGGATGGTTGTG
AGCCACCATGTGGTTGCTGGGATTTGAACTCAGGACCTTCAGAAGCCCATCTCACTAGCCCAGAAGGTGCTAGTCTTAA
CACTGAAAAGCTTACTGCTTTTCCCTATAGTCCTGGGCCCCTGAAACAGTAGGAGCTAGCATGCCATGGCCACCTACGT
GTGCGGCCACACCCGGATGGACAGGAGAATTCAAGCCTTGGAATGATCCTCTTCCTGCCACTCTTGGTATAATTGTCTA
CTTGGACACCTGACAGTCCCCACCCCCCAGTTCAATTTTTTTTTTTCCACAAGTGAGTCAAACCAGAGGCGTATAGGGG
CTACCGTTCCCGGCAGCAGGACTTACAGAAATGGCCTTGATGCAATCCGGATAGGAGGTTTTCTTCACACAGGCAAGCC
GGGGGCCATCAGGCGGAAGGACGGTCTTCATGTGGTCACGGAAGCTGATGCACTTGGTATTCTCGTGCTCTGACACTGC
GCACCATTTGACCGTTTTGTCAGGGACAGCCAGACACAGCCCTGCAGGAGAGAAGGCGGAGGCATAAAGGACCCGCTCC
CCGTGCCTGGGGAGAGAACGCACCACTTTACGAAGGGTCTTAATGGCCACCTCTCCTCTGTTCCACCCTCCTCTCATCC
CTGGTGGCAGTCATGACAGAGCACAGGGCTCTGCTGCACTCCGATCCTGGGAACAGATGAATCCGTTCGCCCCAACGGT
CTGCCATGTTCCCACCTAACCCTGGGCTGCTTAGAGAGATGCTGGGGTAGCTGAAGGTGACAACCTGCTCTTCTTTTGC
CTTGGCTGAAGGTCCACATCAAAATGTGAACTTCAGTGGGCAAACACCATCCTCAGCCACCTTCAGACAAGGCTCTAGA
CACCAACTCTAGGAAAGGCATACCGTCTTTTAGAAATGAGAAAGCTGGCCTTGGGGGAGGAAGCAGGTTTGTCCTACGT
CATCATGTTTGTTAGCAGCCCAGACACTAGGTCTGATGGCCAGGCCCCAGGGGAAGATAACGATCTTAGAAGCTTTGCG
TCTCTCTGAGCGACAGTTTGATCACGAGTTCACCTGTGATACTCTTCAGCTGTCTTCTGTTGAGAAAAGGCTAGGAACT
CCCTTCTATGCAGCAGGCTTCTGTTTCTACCAAACCTTCCCTGGATGCTTTACCCCCTTTGGGCCGCCATTTAGAGAAT
CAGCTGAAGACAATTACGTGTATCACGATGTGTGTATGTGCTTATATAATCGGGCTCCCATGTGGTGTGTGCATGTGCA
GATGGGAACACATGGGGGGTGGGGGTGGGGGACAAAGGGCAACGTGCGGTGTCTCCCGCTATTACTCTCTGCCTGATTT
TTTTGGAGACAGTGTCTTTCACTGAATCTGAGCCTTGCTGGTTCCACTAGATTGTCTGGCCAGAAAGTCCCTGGGATCT
TCCTGTGTTTGCTTCCCCAGGGCACTGTGGGTTCCCTGGGATCTTCCTGTGTCTGCTTCCCCAGGGCACTGCGGTACCC
AGCTTTTTACATGGTTGCTGGGGTTCTGAATTTAGGTCCTGGTGTTTGCATGCTAAGCATCTGCCCAGTTGTACCATCT
CCCCAGCTGGGTCACCACTGAAATCCACACGATGGAGAGATGCCGTCTGTGGGACCTGGCACTCCTAGCTCTTGTCACA
TCCCTTCCCTTTCGGGCAATTCAGCAGGGCTGACTTACTGACTCAGCAAGCCCACAAATCACTAAGCATGGATCGGGGT
GGGTGAGGCCAATGGACCTCAGGATGGATGAGGAAGGACAACAGGGAGACTGATCGTCCCCAGCTCCTTCCCTTTAGTT
CACCGTGGCCAGAGGGATAGACGACCTCTCTAGCCTCTTCAAACTAGTTCGTCTAACTTGTAAATTTTGACCTTGGTCC
GGTGTCACTGTGCATAGCCACACAAGGTGTCCTGAGCTAACAAGCACGGAAGCCTGTTTCAGGCTGACAGTTGTTAGGG
ATGACAAATCACTCAAACTAGACAGCCCCAACACTGTGACAAATGCTAACTACAGTCCAACTGGAGCAGGCATTTGGGT
CTTTGAGACCCCCCCCCCCTCTGGTGAGGAGTCAGCAGGATGCAGGACTGGGACCTGTGACCTCACCCTCACCTGACCC
TCCCAGGCATGTGCGGTAACAAGCAAGACCCGTCGCAGGAGAAATACAGGTATGATTTGAACCCCAGAATTCTGGCTTC
AAATCCCACACCCTAGTCTAAGCCACCAGCGCCAAAGGCATCCAAGGAGGCATCAGACAAAGGAGAGATAGCAGGATTA
GATCTCAGGAAGTTGTGACTACCACACCATGCTGTTTGGACTATACTGAGGCCCCGGGATAGCGCCTGGGTAAACTGGT
TAAAAGTTTTCTAACATCGCGTATACCATATCTTCAAATTACTGGACATAGGGGAGACAGCGTGATGAGGATAGTGAAA
GGTAGAGACTGAATCAGAGCCCCAGATGCATGTGGATCCACGTTGTTCCTACGCTATGGAAGATGAAGATACCACAGAG
AAGAAGCAAGGGGCTGGAAGCTGGGACTTCCAGCTTTAGCTCCTAAGGGTTGCATACATAGCCAGCCAGTCATCCCCTG
CACGCACACTCACCCAGGGCAGCGCAGGCCAGCAGGGCACCCACGGTGAGCCTCATCCTCTCGGTGTGTGTGTGGCGAG
GAGCGGGGAGTACAGACCGACCCGCTTCTGCGCCGCCTGCCCGTGGCCCGCTCGCCTTTATGCACGGAGGGAGGGGGCC
GGCTGGGCGCAATCTTTGACCTCTTGTGTTTGCGCAGTCCAATTGCCCAATCACCCCGCATCCACCCCCTTCCTTTCTT
GCCCTGATGATCACCTCATTTCCTGAGCTGTGGGACAGAACTGCGCATGGAATCCACTACCACGACAATCACACACACA
CACACACACACACACACACACACACACTCACTCAAAAAGCCTGTGTTTAGAAGGAAGCTAAAAGCAGAACCCACTCTTT
GTTATCACTGCCTGGAGCAGCATGGTACAGCTGCTTGGGCGGGAGTGGCTTGCTCCCGGGTGCCAGGGCTTCTACTTGC
CAACAGCTTCATCAACCTTATAGGGGGGCGGGGGGGGAGACTGAGGCTTGAGCGGAAGGGAAACTGCCTAGGACGGCAG
ACTAGAACCGGAGAAGGAACCTAACTGGGAAACTTGGCTCAGGTCAGAGAGGCAGCACGCCATGATCCCAGCAACAGTC
CGGGGGCAGCTCTCCCTCCCTCTATCCCTGCCAGGCCTGGCCTGCTCTGGAGCTGAAAGCTTCCTGCCCTTGAGCTTCT
GGTGAGACAATCCGGGCTAGAAACAGTCACAGGGACAAGAAACTCTCTCCCACTGCCAGGAAGTGGTCAAACTGTGAGG
CTCAGAACAAAGGGCCAGTGTAAGCATAAAGAGGATGCTTCCTTTTTTTTGTTGTTGTTATTTTATTTATTTACATTTC
AAATGTTATCCTCCTCTGAAAAAAACGCTCTATCCCATTCCCCTTCCCCTGCTTCTATGAGGGTGCTCCCCCACCTCTC
ACCCCCTCCACCCCAACTTATCTACCCACTCCTGCCTCACCTTCCTAGCATCCCCTTACACTGGGGCATCCATCCACAG
GACCAAGGGGCTCTCCTTCTATTGATGCCATTAATAAGGCCATCCTCTCCTACATATGCAGCTGGAGCCATGGGACGGT
CCTTCCATGTGTACTCTTTGGTTGGTGGTTTAGTCCCTGGGAGCTGGGGGGGGGTGTCTGGTTGGTTGATATTGTTGTT
CTTCCTATGGGTTTGCAAACCCCTTCAGCTCCTTCAGTCCTTGGCCTAACTCCTCCATTGGGGTGCTAGTCTGATGGTT
GGCTTTGAGCATCCACCAGTCTGATGGTTGGCTTTGGCTTCCTTGGCATCCTGATACCCCTGATTAAGTAGTCACCAAA
CTGCTTTGACTGCATTCATCACAGAAACGTTTCGCTTGCACCCTCCATACGTGCCGCCGTTTCAACTCTCACTAGAGTT
GTGCATTTCAATCCTGTACATGGACTGGAAGATTCTGAAATGATGAAGTCAAAAAAAAATGGGCATTTGAAAATGTCCC
TCTGGGTATATCACCAAGTGTACTATCCCAGGCTCCATATACACTCAGGGAAAGGTCAAGGCGAATCACCAAGGGAATT
TAGGGTTTTCAACAACCGTTTCTTACTGGATTTAATCAGGGTCCTGTCCTCTCTGGTCCTTGGTTTCCTTGTCAAGTCT
GCAGGATGTTGACGCTCTGATTTCAGGCTGCGGAAAGAGCCCGGTGAAGCTCTCAGCACATCTCGGCCATAGGTATTCA
AGAGGAGCCGTGGGTGACCAGAAGCTCTCTGGTGTGGAATTAAACACACAACTGGGGACATGTGCAGATGTGTCTTGGG
CCACCAAGAACACTGAACGTCATGGCGCCAGCCTCTCATCTTCACATCTCCTGTAGACATTCACAGACCTGGGCCTCCC
GTGCAAGCATGTGGCATGTACATCCCCATGGCTAGCCGGGTCCTGTCAACCCCTCAGAAGAGTAGCAGACACAGGACTC
TGTTGCTGTCCTTCTCCTGTGCAAGAACTGGTAGCCTGGACGTCTGTAGCAGGAGCAGGAAGGTGATGTGGGTCTGATT
CTCTGTTTAGTTGACAGAGAGGAGCTCAGGCCAGACTTGGTCGGTCAACATGCTAATTTGGCTGCTTTGGTTTTTGTGC
ACTGGCACTTATGAACCCAAACACAAAGTGACTTTTCCCATTGTAATCCCAGTCTCTGACAGGAATGTTTTAAAGGTAC
GTGTGTGTATGTCCTGGGATGGTTAGAGAGAAAGTTGTGGTATGTCCAAGGGCAAGCACACATGTGTCATATGTCACAG
TGCGAAGGTCAAAGGACAACTTCCAAGAGTCAGCTCTGTTCTTCCACTGTGGAGGAAGGTTCTGGAGATCAAATTCAAG
TTATCAGGTTGAAACTGGTTAGCCTGCTGAGCCATCTCAGCCCAAAGGTCAAATGTTCTAACCTAACACACCCATGTAA
CTGGACATATGAAAAACTTAAACATGGAAACAAAACTGCCAAAAAACCAGGACAGGGACAGAGTGTCGCAAGCATTCAG
AACTCCTTTCCTTTGTCAGTCATTTGCTGAGAGGACCCCCATCTCCCCCCAGGCCCCTCAGAAGCTTCATATCACATGT
TCCTGTCTGGCCTGTGGACCCAGGCGAGGAAACAGGGCCAACCCACACGCTGAGCACCAGTGCTGGCTGGCTAATCTTT
TGCAATAGCAGAAAGTATCACTGGGCCATTACAATAAATGGTGTCCCTGAGAAAGGAGCACTTTGGAGACCATCGGTTT
ATAGCAGGGACACTGGGCAATATGTACATCACTAATGAGGAAGAGAAGCCACAGTTGCTCTGTGCTCTGTCCCCCTGAC
CCTCTTGGGGGTCCTTATGCAGGAAGGCTATATTCCCACTGCCACCCCACAGCCGGCGTTAGGATGGAGTCTGGATTCA
CTGGACACCTGGAGGCAGGAGACACCCAGCAGAGCGATAGATACACATCCAGCGGATGCCTTGGTTAGGCCCTGCAGCG
AGCTTGCAGGGGCTGCCAGGAAGCAGGAAGCAGGGGGGAGGGGCGTTGTGAGGTGTTGAACGTATAACACAAACTTAGG
GAAGTTCAGAGGAGGGCATGGAATACAACTAGAAAGTGACTCTCCCTGCCTCATCCTCTCCCATGGACACATAATGAGT
CTACCTCAAGCTATTCCTCCAGCCACAGGCTTATTAGCAAAGTGTCTGGCCTCACTGGGTCCTCACCAGACCAGCCACC
TCAAGGTCTTACTTCAGAAGGATCCCCTGAGTTCAATCCCAGAGGGGCGCATAGCCCATGGAGAAAGTTCCAGTTTGAC
ATCAGTATCTAGAGCTACCGTGTCATCCACACATACCTGGTAGAGAGGTCCGTGCCATTGGGTCTTAATCAGGAGAAGC
CCCGTGTCTGCCATTCTTCAGACAATGTTTCCTTTTGAGGTGTACGTGTGGATACAACCCTGGTTATATTGACTGCTCA
GGCCTATGTGTCTCCGTAGGAATGGATGTGTTGGGATGGGAAGTTGGCATCACAGTGCTCTGTGTTCTTGGGGGCTGCA
AACATCTGTGTCTGTGTCTTCACTTGTGTGTTTAATTATTCTAAACCAGAGACCCTCTGGATACTCACTGTCCCCCTGA
GCACCTCACCAATGGCTGAGACTTTCAGCGGGGCTTCACCGATCTCTCACTACAGCCCCGGGATTGGTGCTTACGAACC
TCCGTCTAGATATGAGAAAGAACCACAAAGGGATGTGGTAATGCCAGCATACAAAATCAGACACACTGGTTGTAGGTGA
ACTTCTCAACCAGGGACACAGCTTTTTCCAGCAAAGCAGGACTTACAGAGGCCGAGAAGGCTCATCTTTGGTCACACTG
AGAAGGTTACAGGCAAGGATGGCCTCCAGCTCTGCCTTTTTTTTTTTTTCTTTCCCACAGCCATTCCCTCACCTCAACC
ACCTCTGCTGCCAGTTCTGCCCTGGTCAGCCTGGGAAAGGAATGAGTGCCGGGCCACAGAACATCCGATGGGGGCTGCC
TAGATTGTGCCCTGTTGAAAGGAAGCGTGGTTTTAGCCTCTTGCACGTTCCAGACTCTGTTCTGGGGCAAGAGTCTGTG
GAGTTGATCGACAGATGACGTCAGCATCTGTGCTTCCAGACTTATCCAGTCTGTTAAAACGAGGTCTCCTAGTTCACAA
GCTTTGGGAGTACAACTCAGAACTCCTAATGTTAGCCTTCTTCAGACCACAGGGGCTTAATATTTGATATCTCTATTTA
GAAAGAGAACAAGAGGAGAGGAGGGGGCAGATGGGAAGGGGAGAGGCCAAGAGGGGAGTTAAAGGGGGCTTCTTGTTTC
ATGCCAACCTACAGGAGGCCGCAGTCATGTACCACAAAGAGAGTAGAACCTCTCATTTCAACAACAGAGCTGACTTTCT
GGTAGAAACAACTGAACTTGTGTCTACGTGCAGGGGAGTTTGGGGTGCTGCCCGGAATGGGGTAGGCAGGAAGGCAGTG
GGCCTCTGAACCAATATTTGCTGTTTACAAATGTTGCTCAATTTTCATAGAAACTGAGCAAACAAAGAACTTCTGTCTG
CTTCAAGCATTCCTCCCTCCCTCCCTTCCTCCACCCAGTGCAGTGTCCTCTGACCCACCGCCAGAGCCTTCTCTGAAAT
CTCAGTCTCAGCAGTCTTGGGGAAGTAGTAACCTGGAACCCCTCCCCACCCCCAAGGCCTGTCACTGCCAGCCTGCTGG
GTCCCAGAGAGTATGTGAGGCAGGTGGAAACTGGGGCAGGGCCTCGATGGGAAACTGGCGTTCAGATGTGGCAAGCAAC
ACTGTTTCCCTGTCTCTTCCTCCCTCTGTCTACCCTCCCTCTCAGAAGTCCGAAGACAATGGAAGGCTTTTATTCTTTT
CACAGCCAAGAGCACACATAGTAGCATTCTCGTAAACAGTAACAGTCCACTGCCAGGTGGGCTGAATTCAATCCAACAT
TTAAGAAAAACTGCAACAGTCTTACACAGGCTTCCTGGGAACTAAGTCTAGGAAAGTCCGAACTAATGTTTTGAGGACA
GAATGTATACTTTGAAAGGAAAAAAAAATCAATAAATATATCACAACAAATACAAAATATCTTCTGAGAACACAGATGC
AAAAATGCTATGCAAAAAAAATTTCATAGCTGGGCAGTGGTGGCATATGCCTTTAATCCCAGCACTTGGGAGGCAGAGG
CAGGAGGATTTCGAGGCCAGCTTAGTCTACACAGTGAGTTCCAGGACAGCCAGGGCTACACAGAGAAACCCTGTCTTGA
AAAACAACAACAACAACAACAAAAATCACAAGTAGGGTTGAGAAGTGGCTTGGTTGGTAGAGTGCTCGCTTAGTGTGGC
CGAGGCCCTGGATTTGATCCCTAGCAATGCATAAATGTGGGCTTGGACACACACCAGTAAACCTACTATTCATGTTAAC
CTTGGCTACAAAATGAGTACCAGGCCAGCCTGGCCCGTACAGGAATATCTAAAAAAAAGTCACAAAGAGGATCCACTAA
AATATAGCAAAGATAATAAACAGTTACTCTCAATTTGACTCCTCCAGGAATGCAAATTATCTTTTGAAGCATCAATGTA
ATTGAATAAATTAGTATAATTGTGGAGGAAATTAATAACCACATCTATCACTATCAGAGCATCATTTGATATAGCTTAA
CATCCACACTTAATGAAAGATCTTAGCAAACTTGCTCCTGGATTTGATGAAGGGTATTTAATAGAGATCTACAGATATC
CTGCTTTGTGGTGGAATGTGACATGAAAGCTTTCTCTTTGAGAGCAAGAACAATACTAAAACATCTACTGCTATCATCT
CCGCTGAACGTATTAGTGGCGGCAGGTCTGAGCCAGACCTCTAGGCAAGAAAAAGAAATAGGAAGCAAAATGACTGAAA
AGAAGAAACAAACAAAACCTCTTATTTTTATAGATGGCTTAATTGCATATGTATAAAATTCTAAATACATTTATTTTGC
TTTGTAGGATAAATGGGCTAAGAAACATCAGAATGGAAGGTAAATTATACAAACGCAACTCATTGCTATATACAAAATC
TAAAAACGAAAGAATTCTACCTAAAGGACAAACAGAGGCATCGGGATTGCCTTGCACAGAATGATTAAGTCAGTGAGGA
AAATAAACTTGAAGTGAGCGGGAAAACTAAAGTGTGCAGCGTCAGAGCCAGAATTAGGAAAGTCGTCGCTTCTCTCCAA
ACTGGTCTGTAGATGGGGGTGGGGCATATTGGGAGTTAGAGGTTTAATCCGGGATGGGGCATGAGAACAGGGCCTAGGA
GGAAGTGATGGGTAGTTAAACTAAGGGTGTGTGGAAAACGCTATGTAGAACGACTACTTTGTAAGCCAATTGAAAACAC
ATGACTTTCTAACAAGGAGTTTGGGAACAGGCACCCTGCTTTGGTGGACAATGCTTCTCTCCAGATGATGGCTGCTATT
AAATGAAGATCTCATTGCCAAGTGTGGGATCCCTCTCGATGAATTATTTGGTCAAAAAAATAAAAATCCCCCAAAACAG
CACGGGCTGTTGCCATTGCTTTTGATCGTACACCATAACCAGCTGGTAAGACCCTCCTGCTGAAAATATTTTGGTTTCA
GGACATAGAGAAAATAAGCTGAAATTGGCCTGAAAACTGTCTTCCTGCTGGCTAGCTTTCACTGTGCTGGAGGGTGCTA
CAAAGGCCACTGGGGGACGAAAAGTCATCAGTGTGGCTTACCTAGCAGTGAACCCTGAATGCTCCAATATTGACCCACT
AGGCAAGATGCACCCCTAGTGTAATGGTGCCATTAAGGTTATGGTGGTAACCAGCTCCTTTCAGATTGGTTTGAAGCCT
CCACCGAAGGGAATTCACTCTTCATGTTGTAAACGTGGTCCAAGGGTCAGAAGGTTGCAGGCTATCGTGGGGCATACAC
TGCTGCTGCTTTCCTACATGAACATGTCAAGCTGTCTTTTAAGGCCTGCGAGATGGCTCATCAATCAAAGGTGCTTGCA
GCCTGACAACAGGAGTTTGATCCCCAGGACCCAACCGGTGCAAGGAGAGAATCAGCTCCTACAAGCTGCCCCCTGAGCA
TCACAGGCAAGACTTAGAATGCACATTTACACACACACACACACACACACACACACACACACACACACACACACACACA
CACACTGGATGGATGAATGAATGAATGAATGAATGAATGAATGAAATTTTAGAAATTAAAAAGCAGAGCCTTGCCTTCT
AAACAGTGATGGTTATCCCCAAAGGTTAGTTAGTGCTGCTCTCTCCCTTGGTCAGAAAAGCCTCTTGGTACGCGGAGCA
GCAGTGAATACAGAGACTCACAGCTGATTAAAGTGCTGAGCGTATGGGACTGTTGAGTGGTCAGCCCCAAATAGGACAT
TTATATCAATCCCTCCCTCAAGGCTTAGGGAAAATGGTGAAACGGCCTAGACTGTAGTAGCCAACCACTGGAGAGGGGA
GGCTGCGAAACACTGTCTCCTGGAGATGACATGGCTGCTGCTCTCATGAACTCACCAAAACTGTGGGTACCTGTGTGCC
AGCAGCATCAGTCAACTCCCCAACAGCCAGCACTAGTTAGACTCGATGTGTCACAAAATAACAGTAATAATAACCCTAA
CACCAATAGAAGACGTGAGGGTGAGGATATGTTGGGGGTGGTGTCCGTGCTGTGCTGTGGGCAGGGGAGTGGGAGAGAG
GAGGTTGGAGATTGATATTATCAGATACACTGTATACCTGTATGGGATTGCCAAAGAATAAATAAAAGAGATTACTTTA
AAAATGACTTATAGATTCCAGCACGTGAAATGTTTGCATGTCTTTGATATAAAATGACCATATATTTCCACAGGTCACA
TACAAATACAAAAGTCTCCATGCGTTTGCTGGTTGTACAAGGCAAGGCATACAAGATAAGAATAGATTGCATTTAGGGC
TTTTTTTTTTCTGACCTCCTTGCCCAACCCTGCACCAAGATCTATAGTATAACCAATTCAGGTGGTGATGGTGCATATA
GTAACATAAGTTGTTTCTAGAGTGCTCTTGTACTAAAGGCAGTGAACAAAAAAGACACAGTCAACCACACACCCTTTCA
AAAAATATTTTGTATACTTTCTGAGATTATAATTACATTATTCCACCCTTCCCTTTCCTCTCTCCAAACTTTCCAATGT
ACCCTTCCTTGCTCTCTTTCAAATTCATGGCGTCTTTTTTTCATTGATTGTTGTTACATATATACCTGTGTATTCACCT
GTATTCCTAAACACACACATACAGGCTGCCTAGCCTGTATAATGTTACTTGTGTATGTTTTCAGAGCTGGCTGAACATT
TGGTATTTGATAACCAGTTGGTGTGCTCTTCCCTGGGGAAGGCTATTTCTCCTGCTCTCAGCAATCCTTGGAGCCTAGA
GCTCTTTGTATAAGGTTGAGGCCTTGTGTGAGCATGTCTCTTGTTCTTGTCCTTGTTTGGCTCACTCAAGCTCGTTCTT
AAGGGTGTGTACCTACACATCACCGCAATGGCTAGTCGCTATTCTCACTCCCACTTCCTACATTAAGAAGGAAAATAAA
CTTTAGATTTTACTTCTGGGGTTTGAAGTCCCTTTCAGATACTTTCCCTTCCTTCACATGAGAGTTTTCAAATATCCAC
AAAAGTGAGTCAGTAACATAGGAAACGCCCACAGACAACCCCCAGTTCCAACAATGCTCAGTGTTTTGTCTATTTTCTT
TCACCCGTCTTCCCCCTTCCCCCCTCCTTTTTTTTTCTGAAGTGACAATTATCTTATTTCCCCCAGTATGCGATATATC
TTTAACACACAGGAAATTTTGCATAACACTACTGACATCTATAGAAATTGATGTTGATGCTCTATTACCATCTATTTGC
TACTCTCCAACATGACTTTGTTATTTCTTTGTTATTTGTGTCTGCATGTGTGCACATGTGCTCGCTTGCAAGTATGGAG
GCCAGAGGACAATTTTCAGAGTTGTTCCTCTCCTTCCACTGTGGATCTTGAGGTTGGACTCAGGTTGTCAGACACAAAG
GGAAGGTCTCCTAGTTGCTGAATGGTCACCAGCTCCCTTATTGCTCAGTCAATCTACAAATGTCTATGCGGGTCTAAAA
GCAAATTAAACTAGGAGCTCAAAGTAGATCCTAAATGGGTTCTACATGCTACGTGAGGTCTATCGAGATCTGCATGCTA
CATGAGGGCTATCCCAGAGTCTCTTTTGCTCTAAAGTGATCACCTACATTTTATGCTGTTTACTTCAATAAAGTATCTG
AAAGGGACTTCAAAGAAACCAGATCTTTGTCCCGCAGAATTTCCAGTGTTCCGGATTTGGCTGAGCTGTATCACGCTGG
TGGGATTTAGCGTGCTCTTTTGTATTTGACAGCTATTTCTGGGGAGAGTTCCTTGACCTGGGTTTTTCTGAGATTACCT
CATCATGAGACTTTGATTTCCTGATTCTGGCAACTACCTAGAAGAGGTGACATTGTATCACATGGCACAGGCTGCCATG
GTGACTTGTTGCCTTGCTGTGTGGTTAACTGTTTCTTGGTGAGGGTATCTGGTAGGTTTTACCACCGAGCCCTGTTTTC
AATTAGTAAGAAGTCTCCAAAGAAATACTTAGAGACTATTTCACATTTTTCTCCTAGTAGGATTTTTGCCCGCTGGTCT
TAGCATTCAGTGGTGATTCTTGCCAGAATGACTTGGTCCTGCACCGATAACCAAATGTTGACACTCTGATCCTCTCATT
CCCTCTAGATTCATTGATGAGCATCGTAAAGTAAGGAAAAGCCTCCCCTTCACACATTTATCTATCATTTAGATCAGTG
TGGGCTCATGGGTTCCGATTTTATGCTGTAGATTATAACCCAGAACATCACTAGTCACTTTATTTGTCTCTCAAGGATA
GTTGCTTTGTTTATATATACTTACTTAGTCATTTCACACCTGCATGTGTATATTTGTGAGCACCCGTGTGCTGTGGCAG
TGTGTGGAGATCAGACGACAGTGTGGATTCCTGGGGCTGAGCTAGGACTATCAGGCTTAGTGGCAAGTGCATTTACCCA
CCAAGCCATCCCTCCAACCCTTGTCTTCTCATTATCACCCCTCTGTTGCCAGGACAGACTGTGTCCTCTAAAAAAAAAA
AGCCCCATCAATCAAGTCCACCTTGTGTTTTCTCTGCACCGTCTCCGGGGTTTGTGTTTATGGAACCAAAAACTCCATG
GAGACTTAATTAACCTTGTAGTGGAGAATGGTGTTCAGAAGTGCATTCTGAGAGCCTGCTGCTGAGGACTCATAGACCA
CAAGACTCTTCTGCACACACAGCTAGGATGCACATGTATGTGTGTGTGTTTGTGTGCACCCGCTTGTAAGTACACACAT
AGCTGTACCTATTTTGCATTCTGTGTGTGTGTGTGCCTTCTCACTGTTTCTGTCTTCCTCTCTGGCAACCATGAGTTCA
CACTGACTGACTGAGCGAGCCCCAGTTAAACCACAGTATGCATTCTAGCTTTCTTCTCTCCATCATTGCAGGGAGAGCT
CAATGCAATTGAACCATAACCCACCATCTATCTAGATGTCTCCTTCCCCGTCCTATCAGTGAGAACTGGGTACTCCCAT
CCCCCCATCTCACTCTCCTTTCTCTTTGCTTCTGTGCATCATTATTCTCCTTATCCTCCAGGTCCCTTCACAGCCTCAG
GCTCTTATGTGCTTCAGTCTCAGCCACTCCAGGAGAAGAGAAGAGAGGAGAGGAGGGGAGGGGAGGGGAGAGAAGGGGA
GGGCAGGGGAAGGGAGGAGAGGAGAATGGATCCAATTTTATTGTTTTTTGTTTATTTCCAGTAAAAGGACAAGAGAAGG
AGAGGGAAAGGGAGGAGAGAGAGGAAGACTATCTTTTCTCCACAGTACCCAGAGGACTGTGAAGAGCAGAGCAGAGCCT
CGCAGAACCTTCCAGTGAAACAAAGCTTTGACTCCTTTCCACCCATCCAGTCATTCTGCTGCGGTCAACTAGGAGAGAC
CAGAGCTGTATGTGAAAGCCCAAGACTTTCATTCTGCAATGGAGGAGCTGCTAAGAAGTTCCTTTCCTAGTCCAGTACA
ACCCAGCCAGGCTCTACCTAACTCAAAAGCCAACTAGAGAGGCCACTGACCCCGGAATTGAGTGGACTCTGTCAAGTCT
CATGTGTAAAGGAACAAAAGTCCCTGACCAAAGCGGGGCCTGCAGGAATGCAGGTCTTCTGTGGCATGCTGTTTACCAG
ACATCTGGACAGACCTGCAAAAGGTGAGGTTAGAGTCCAGGGTCACAGTTTAATGTCCCACAGTATGGCAGATTTGGGA
AGGAAATGCAAAACAGTGACAGAGACACCACATCTGTCACAGAAACTTCTTCAGTCCCCAATTCCAGGGATTCCTCTCC
CAGGGGACAAAGTAGGAAGCTTTGGCCTGGAAAGGAAGGCAGGGTATTCTGAGAACTATAAAGTAGGCCAGAGAACTAG
ATGGCAAAGAGTCTGGAGTTTCAAACACTGGGAGATTCTCAAATAGAGAAGTTGGCTTGCAGAAGAAGAGTCAGTCCCA
GATCCTGATTTGTAGAAGGTCTTCACAGATGCCATCCTGATAGAAGAGGAACTATTCAAACATTGGAGGAAGTCCTAGC
AGGAAAGACAAGATGGGGACAAGAGGTCACATCGTGGTCCTCCCTGCTAAAGGCACCCTTAAGTCTCCAGCGACTTAAG
TGACTTGATCATATAAATGAAGACCTGTAGGGTAGATTATGAACTAAGCAAGTTAGTTAAGCCCCTCCCTCCTGTGGCT
GCTCCTCATCCAGTAGGAGGATTGGATTAGTAATGCCTATCCTAGCCAGTTGGAGAGATTAGTAATGCCTACCCTAGCC
AGCTGGAGAGATTAGTAATGCCTACCCTAGCTGACTGGAGACATGGCTAATAAGTTGGTAAAGTGCTTGCCGAGCTGAC
ATGAAGACCAGAGTTCAGATCCCGGCACCCATATAAACAGCCAGGTGTGGCTGCTTACAGCTGTGATCCCAGCACTGGA
GAGCAGAGACAGAGGGTTGTCGGGCCTCACTTGCTAGCCAGACTAGCTGAATTGGTGAACTACAAGCTCAGTGAGAGAC
TCTTGTTTTAAAAATATAAGGTGGAGAATGATGAGGGAGACACCCAACATTGATCTATGGCCTCTACACACATACATGT
GCACAGCAGCTACATACATACATGCATACATACATACACACACACACTCACAATGTGCATGCATGTAATCCTATCACTT
GGGAAGTAGAGACAGCAGAATCAAGAGTACCAACCTAGTCTTAGCTATATAACAAGTTTGAGGCTATCCTGGGATACAT
AAGACCCTGTTGTGTGTGCGTATGAGTGTGTGTGTGTGTGTGTGTGTGTGTGTGTGTGTGTGTGTTTTGCCTAGTTAAT
AGAGGATCTAAATTAGTGAGGTGTCTGATATGGGTACTAGAAAATGAACTAAGGTCCTCTGGAATCTTAATCACTGAGC
CATCTCTCCAACCCCGTTTTATTCTTATTTTTAATTATATGGTAGAGGCAGAGGGTTTTCATGTGAGCTAAGGCAACCA
CAGAGGTCAGAGGTGCCAGATCTCAGGATATGTTAATAATTGACCTAATACAGTGTTGATATATTGCTATTTTTTAAAA
ATTTTGTCTATAATCTGACCAATATTCAGCACCCCCCCCCTTAAGTCCTTTAGGTACAGTCTTCAGAACTTAGCTCCTC
AGGTCCAATTTCTTGTTTTGGTGGTTATTTTTATTATTGTTAGGGTTTTTTTGCTTGTTTTTTGAGACAGGTTCTTAGT
ATGTATCCCTGGCTAGCCTGTACCTCACTTCGTAGACTAGGATGGCATTAAACTCACAACCACTCAACTACTTCTTCCT
CCTGAGTACTCACCTCTCAGGTTCCATTTTATCGGGGAACCTTTTAAAAGGTGCTGATCAATAGATCCTAGGCAGTGCA
CGCTAGAAGACTTAGCTAGGAGTTGTTTGTGTGTCTGTATACAAATTACTGGTGACCTCCATCTATACATCTAACCTGG
GCTGAGTACCATCTTGTGCCATCCAAGGAGTAATAAAACATATAAAGACTCTACTTCAATCCTAGGCCACAAAACAAGG
TATCTAAAAACAGGCAAGCTTTTTACTCTTAAGCCCCACTATCCCATTAGTGATAGAAATGCAAATATCTTCCCTGTTA
TGACAGCTACTGACCTTGCTAGTGAGATGCTGGCTCTGAGCTGACAGGTGGCAATCATGATCCCCTCCATTACTACTGG
GCCTGTCAGCAAGACACAGGTAGGGAGAGAGCATCTACTATAACAACCTCACTTCACAGATTAAAAAAAACAAAAAACA
GAAAACTGGAGCCAGGCAGTGGATATAGCTGGTCCACTGCCCAGTAGAGCAAGGACACAGGAGCCTAGAACTGGGCTGG
TGCCCTGAGATGTCCCCTTTGTCTTGTTCCTGGAAAGTACCTCTCATTTCCCCCAGGTTTATTTCAGAATTTAATTCTC
CCTGTTAGTTCGTCAAGAGATGTTAAGACTCGGACTTTCAGGTGGACTCATAGGCAGGTGACCAGATAAACTGAGCAGA
GTCCAAGGCTCCCAGGGCCAAGGCAAACAGAACACTGGCCCCTCTGCTGTTCCTACAGCCTATGCTGTTGTTGAATGTC
CCTACTTGCCATGGAAAAAGAGGACCGAAGTCCACCTTCCTTCCCGGCTTGTCCAAATTAAGCCTCTGACCTGTACAAA
TTCCCAGACATACAAGCCATCAGTGTCAACTGTTGGTTTCTTCAACTAAATGGCCTTATCCTTTTTAGTTGTCAGGAAT
GTTACTCATGGACAAATCTTTAATCACTATATAACTACTCTGTATGCGCAATTTAACAAAACCTACCAGCCTTTCAGCT
TAGCACCTAAAATGAACTAACAAGCCCCTATTCCCATTCTCCTCCATGAGGAAGGAAGGGAGAAGTCTCGGGAGACCCA
CTGAATTTGAGACACAGATGCCAGAAGCTATATACAAAGGTGTGTGTGTGTGTGTGTGTGTGTGTGTGTGTGTGTGTGT
GTGTCTGTCTGTCTGTCTGTCTGTCTGTCTGCATGAGTGTGGGTGTGTGTGAGACAAAGGTGAGCCTCAGATTTTAGTA
ATCCTCGATAGGATCCATCAATTGGTATTCAATATGTCAAAAATCTCCACTGCTTTTACCAAGATCTTAGGTTTGGGAG
AAGTTGAAGACACTCGACAGTGGTTCCCAGTGCCAATAAAAGACACCAGTGAAGCCTGGCGTGGTGGCGCATGCCTTTA
ATCCCAGCACTCGGGAGGCAGAGGCAGGCAGATTTCTGAGTTCGAGGCCAGCCTGGTCTACAAAGTGAGTTCCAGGACA
GCCAGGACTACACAGAGAAACCCTGTCTCAAAAACACCAAAAAAAAAAAAAAAAAAAAAAAAAGACGCCAGTGGAACCA
AAGCTCAAGATCTTGCCCCAGTTCTGTGTCACTTAGGACCACAAGGTTGACTTTCCTGGTGGAGCAAATGGTAGCAGAA
ACTGAGTCAATATTGTCTAAGGCTTTGGTTCATAGCCTGGAGATTTTGTAGCAAGTGAAACTCTGAAGGAGAATCTTCT
AGAACCCGTAGCATAAAAGACTGGCGTGTCTGTGACTTCCTGGGGCAGTCTTTAGGGAGATGGCATAAGACCCAGAAAG
AGTAATTCCCCTGCTCCAGAAACTACCTGAGCCTGACTTAGTCAGGGTTTCTATTGTTGTGATAAACACCATGACTAAA
AGCAACTTGGAAGGGAAGGGGTTTATGCCACTTACATTTCTAAATCACGATCCATCAATAAGGGAAGTAGGGCAGAAAC
TCAAGGCAGGAACATGGAGGCAGGAACGGAAGCAGAGGCTGTGGAGGAGTGATGCTTACTGGCTTTCTCTTTATGGCCT
GCTCAGCTTACTTTCTTTTTTTCTTTTTCTTTTTCTTTTTCTTTTTCTTTTTCGTTTTCTTTTTCTTTTTCTTTTTCTT
TTTCTTTTTCTTTTTCTTTTTCTTTTTCTTTTTCTTTTTCTTTTTCTTTTTCTTTTTCTTTTCCTTTTCCTTTTCCTTT
TCCTTTTCCTTTTCTTTCTTTCTTTCTTTCTTTCTTTCTTTCTTTCTTTTTTTTTTTCGAGACAGCGTTTCTCTGTGTA
GCTCTGGCTGTCCTGAAACTCACTTTGTAGACCAGGCTGGCCTCAAACTCAGAAATCCGCCTGCCTCTGCCTCCCGAGT
GCTGGGATTAAAGGCATGAGCCACCACGCCCAGCTTCAGCTTGCTTTCTTATAAGCACCCAGGACCCACCAGCCCAGAG
ATGGCACCATCCACAGGGAGCTGGGCCCTCCCACGTGAGTCATAAGTCAAAGCAATACACAACAGGCTTGCCCCCAAGC
AATTTAGTGGGAGCATTTTCTCTGTTGAAGTTCCCTCTTCCCAAATGACTCTAGCTTGAGTCAAGTTGACATGGAATTA
GTCAGCATAGCCTGAGATCTTTCTGCTACCTCTCTGGGGACTGAATTATAGGCTGGGGAACGATCTGACCCATAGTCTT
CCAATGTTTACATCGGCTCTGATAGTTGAATGATTTGGAAAAGAAAACCCCGTGTCTAGGGTTGACTAGATAAGGACCT
TGAGGCTGGAGCTCAGAAGTTGGGAGAACTTTTGGAGGATTCTATCTTGAAGCCATCGATTCCTTCCAGTTCTCCAAAC
CAGGCTAAACCATGGCTTTGGCTGCAGCTTCTCAAAGCTGAAGTGCTCCATTTTGTGGATTTCTGGCCCAGGAATCTTC
ACCATAATGTATGAAGTTAAAACTGCCGGCTTTGGAGCAAGGAAATTGGAATGAATTTGAATATAAAACTCTACTAGAG
GGGGACTGAAGAGATGTCTTGGTAGCAGTTAAGAGCACTGACTGCCCTTCCAGGGGACTCAGGTTCAATTCCCAGAACT
CACATGGTGGCTCACAACTATCGGTAACTCCAGTCTCAGGCCATCCAATATCCTCTTCTTGTTTCTGCAGGTTCCAGAC
ATGTATGTGGTGAACACACATACATAGTAGCAAAATACTCATGCACATTTTCTTCTTCTTCTTTTTTTCTTTTAAGATT
TATTTATTTATTACATGTAAGTACACTGTAGCTGTCTTCAGACATCCCAGAACAGGGCATCAGATCTCATTACGGATGG
TTGTGAGCCATCATGTGGTTGCTGGGATTTGAACTCAGAACCTTCGGAAGAGCAGTTGGCACTCTTAACCGCTGAGCCA
TCTCTCCAGCCCTCATACACATTTTCTAAAGCTTCTAGAAATGTTTATAGGAAATAGATGAAACTGGAGAGTGATAGGT
TAAACTAAACTCGGACAAAAAATCACGTTTTCTCCTATATCTAGATTATAGATTTTTAATGTATCTGTGTATTTGGATG
GACGAGTGTGTGGTGTGGGGTGTGTGTGTAAGTATGGTGCCTGTCAAGGCCAGAAGAGGGTGCTGGATCCCCAGTACTG
GAGTAACATGCATTGTGAGTAACTCTCTGTGGGTGTTGTGATTGAAATTCTGTCTCGTGACTGAACAGCAAGCACTCTT
ACCTGCTGTTCTGTCTCCCCAGCCCTGCTGCTTCTTTCTTACCTTATACGTTTTTCTTTGTTATAGTCACCGTTTTCTC
TAAATCCTCAGATGTGTTTATAACAACTCTTTCTGGAGTTGTCTGGAGACCAACACAAGACTGGTACATGGAACCGTGC
TGATAAAGGTGCCTGCTCCTGGGTGTGTAGTCTCCAAAACTGAGATAACAAATCCCCTTTCTTTTTTTTTAAATATTTT
ATTTGCATTACATTTATTTTTAAATTTTATGCATTTGTATTCATTTACATAGCTTCTCCGTCCTCCCTCTAACCTCTTC
AAATCCCAGATTCTTTTCATGGGCCCTTTACTTAATATTATTGCATATATATGTGTGTGAATAAATATATAAGTACCAT
CTTCTGAGTCCATTTAATGTTGCTCGTATATATGTTCTTTAGGGCTGATCACTGGTATTGGAGAACAAGTTAGGGGACT
CATCTCTCTAGAAGAATAATTCTTCTCTCAGTACCCATTAATTTCCTATAGCTCTTCATCTAGGCGCTAAGTCCCAGAT
TTCCACCACCAGCATTAATGTGCCATCTAGTTGTTGTCTTTGCCCTGGTCCTGCTTAGGCAGTCATAAATGTTGACATT
TCACGAGTACAGCTTCCCTGTCATATAGAAGACACATATGGAAGGTATAGTCTTAAAGATGCTTGTTCTTCCTCTGGTT
CTTATAGTCTTTGCCCTCTCTTCTTCAATGTTCCCCAAGCCTTCAGTGTGGGGGTCATGTTGTAACTGTATCAACTGGG
GATGGGCTCCTCACAGTCAGTTGTTCTGTTCAGGTTGACCAGTTGTAGCTTCCTGCAGTGGTCTCTATCCACTGCAAAA
GGAAGCTTCTTTGATGGGGAGGAGGAACTATATGTGGGTATATGGAGAATTATTTAGAATGTAGTGCTGGTCTAGTTAA
AGTGGCTGTCATAGGTTCTCTAAGAACCAGAACCTCACTAGCCTTGTGTATTTGGCTAGATTTCCAGCACCAGGCATGA
ATGCCCTCATTGCTTTGTCTAACAAGAAAAGATCATGCTGGAGCAATGGCTCATCAGGAGCTGCTCTTGCAAAGAACCC
AACTTCTGACGCTCACAACTGCCTGCACTCCAGGTCCAGGATGTCTGCTGATCTCCTCCTCGTCTCTGTGGGCACTGCA
TTCACTCGTGCAAGCCCACTCCCACTCACACATGTGACACGTAATTTTTAAATTCTTTTGAAATTAAATCTTTTAAAAA
GGTTATTGTTTGAATTCCCTCATCTCTGGGTTTTAAACGTGCTTTGGTTGGTAGACCTCGCTCCTGACTTTTTAACAGT
TCAATGATTCCTTGCATATCTGAAGATTTTTTTAGTTAAGATGCAACTAGGTGAGTTGCCCTGGCAAGTACTTAAATTT
TGGTTGCCTTGGCCTCATGGTCTTGGATCTTGTGTTGGAGCATAGTTAAGATGCCCACAGACCAGTGGGGTCCTTTCAG
GGCAGTGCCAGCTCCACTGGAGGCTGGAAGGAGTAACCTCCAATCAATGGCCATTTAAAATGTTCGTGTGTATGTGCCT
GTGTCTCTGTGGGTTTATGTTCACCATATGTGTGCAGGGACCTGCAGAGGCTAGAGGGTGTTGGATCCTGAGGAACTGG
AATTACAGGTGCTTGTAAGCTGCCTGAAGTAGGTTCTGGGAACTGAACCCAAGTCCTCTGCAAGAGCACTGAGTACTTT
TAACTGCTAAGCCGTCTGTCTTGCCCTCTCAATGGCCATTTAACCCCACATCCAAGGCACACCCTGTAGTGAAAACCCT
TTGTGTTGAGCGAGGGCTCTTTGCTCTGGTTGCTAGAAGCTTGCATGATTGATGACCTTAAGCCCTGGCAGCTGTGTGG
GGTTTAAGTCTCTCTGTTACCCTCTACCCCTCTTGTGGCTTTTGATCTCAGGCTACACCAGTGTAGGTACATCAAGGAC
ACTGGCATGCGGATCCTGGAGAGTTTCCTGGCAAAGTCCCATCCTTTTTGGCACTTGATCCCAGTCACATTGACCTCAG
ATCACTATCTTTGTTTCTAGAAACTTCAGACTGGCAGAAGTCCAGTCATCATCTTCTCACCTCTTCTTTCCCTTCCTCC
GTGGACGCTAGTCCCTCACTGTCATTGTTCAATGTCCACAGAGAGCTGTTTCATACGTTCCCTCAGATTTTTAGCTGTT
TGTGGAAGGAGGGTATGCCCTGCCCCAGGCACGCATTCATTCACCCAAGTGAGTCTGACTTTTAACAACATGTCTTGGC
TCTCAATCTATGCTTGCTAAGAGGCTGTCGAAAGCTGACATCCTGCTACTAACCAGCAACCTCGAAATCTCTGATAAGG
AACTTGTCACATTTTCAGAAAGCATCCTGCAGATGCAGTAAGATACACTGTTGGCAGTGATTTTGATATTTTTCAGGAA
ACATTAACTAAGACTTTGATTGTCTTTGACATTAATTTCAAGTGTCAAAACATAATTTGTTTTACTGTATTTCTATGTG
TCAGGTCAGGGTGCATGTGAATAGACACACATGCACACACACACACACACACCACACACATGCGTGCATATGGAGGCTG
GAGGTAAACTGGAGGTGAATGTTGAATACTGTTCTCTACTGCTCTCCACCTTCGTTTTTGAGACAGGGTCTCTCAAACC
TAAAAGGCATTTGGCCTGGCCACCCAGGAACCCTGGGGATCCTCCCATCTTTGCTTCCCCAGCCTTAGGATTACTAGTG
CACATCACTATACCTAGCCTTCTCCGTGGGTGCTGGGATCCAACCTCAGGCTCCTGTGCTTGTACTGCAAACACTTTCC
CAACTGAACCACCTTCCTGACCCCCAAACCATGCTTAAAAAATACACAAAATCATCCCTTGTCCTTTCTTGTTATTATG
TGAGTCATTTCTATTGAGCATTGATGCGATTGTGTGTGTCTGTGTGTTCTTATATTATTGCTTGGATGGTATTTAGGCA
GATTTTTTTTTTTTGCTCAAACTATAATTGAATTAGTTATCTCTTGTGTAAAATAATCAGATTATAAGGAGACAAGTCA
AACTAACCTGGTATAATTATTTTAGGAGTACATTATTTTTATTTTGAAAGTGTGTGTGTGTATGTGTGTGTAAGTGTGT
GTGTGCAATGCATGTACACTCACATGTATACGCATATGGAGTCTGTAGTTTGACACCAGGTATCTTTTTCCATCACTCT
CCCCTGTATTTTTTGAGAGAGGGTCTCTTGAGGAACCTGAAGTTGACTGTTGCAGCTCTGCCAGATGACCAGCTGGGTT
CCACCATCTCTGCTTTCAGCATTCAGGTTACAGAAATATACAGCCACAATCAGGTTTCACTCGAGTGTTGGGAGTCCAA
ACTTCAAACTAAGCATCAACCCAAGGGATAATCTTCCAAAGGTATAATTTTACACTTATTTATTTGTGGATGTATGTGC
ATGGTTGCACACTCATGTTGCTATGTCCGTATGGAGGTCAGAGGACCACTTGTGGGAATCTGTTATTTCTCCTGTGTGG
GTCCCAGAGCTCAAACCTAAGTTGTTACATTTACCCATCTCACCAGCTATAAGATATAATTTTTAGTAGATACTAAGCT
CAGAGTCACAATTTTAATACAAAAAGTCTTATTACCTTCAAATCTGAGAACACTCAGTGAAGCTTTCTGGGAAGATACT
TTTATTCATTCTGCACAAGTCCACATGGAGGCAAGCAGTTGGGTCCCCAGCCCCTGGGTTACCGCCCCCCCCAGCTGAA
TGGTGTTTAATATTTATGAAATGTGCAGGCATCCAGCAGTTCTGTAAATTAAAACACAAGGGGAAGAACATCAGGCTCT
GCCTGCACTTGCCAAACTCGTCTGCTCTGATTTCTCATGCTCTTAAAAGTGAAATTCTTGAAAGTGTATTGGAAACACT
CCTGTCTTCTAAAAGTAAGCCACAGTTGAGATGAGTTAAGACCCTCACTGGCCATCTCTGTCCTACCATGTATATCCTT
AAGTATCTGTCCTTCCTTCATGTCCTTAAATCCCTGTGCTGTCCTTCCTTCCTATCCTTAAGTACCTGTCCTTCCTTCC
TGCTTACATTATCCCTAACACACAACACAATGCCACAGAGTCGGGCATATGCCCAATGGTTATCTCAGTTTTAATTTTA
TTTTCCCAATTTCTATTTTTTTGAGACATCTATGCCATCTAAAATATGTATCCCAAGATTCCTACAGAGCTAGCCATGC
TGTATTTACTTTTATTAATGTTTGAGCATTTTATATAAAACCATCAAAAACCAGCCCTTCACGGGTGGTATGCACAGTG
TGGTGGTTTTAATATACTTGGCTCACTATCACAAGGTGTGACCTTGTTGGAGAAGGTGTGTCACTGTGGGTGTGGGCTT
TGAGACTCTCATCCTAGCTGCCTGGAAGTCAGTCTTCTCCTAGTGGACTTCAGATAAAGATGTAGAACTCTCAGCTCCT
CCTGTACCATGCCTGCCTGGATGCTGCCATGTTCCACTTTGAAGATAATGGACTAAACCTCTGAACCTGTAAGTCAGCC
CCAATTAAATGTTGTCCTTATAAGAGTTGCCTTGGTCGGTGTCTGTTCACAACAGTAAACCCTAAGACATACAGCTTGT
ATCATTTCCCAAGCCCCAACAAAAATGGGAATACACATGAGAAGAGCTTCATTTCCCAAATGGTTTCAAGTTCTGCTAC
ACTTCTTGTCTCCTTCAGACAATTTAGCATTACATGGATGGTGTCTTTCATTATTTTCTAATTGAATTGGCTCAATGGT
AACTCTAAACCTTCAAGTCAAAGAGAAAGTTTCTGCTTCTCCATGCCTTTGTATCAACTGAATACTTGAAACAACCGAG
CTCTTAACATAAACATGCTGATTTCGAATCTCCTGCTATTTCTCCTTGGTTGTTTCTACTTACCAGAGGACAAGCACTG
TCTAAAGTTATCCATCAAGGTAAGATACTGTGGTCCTAGGTATGTTTTATATGTTGTTTTGTTCTGAAGGTTAGATAAA
CATTCTGTGTCATCACTGAAAAGCAGGTCCTTAGCTGAGGATTCAAACATCTGGAACATCATATATTCAAATCCATTTC
TTCCAAAGAGCTCCTGGATTGGAATAAACAGAAAGTCACTAGGGGTGCCAGGAAGCATAGAGGTGTCTACTCTTTCTAG
AAAACCAAAGGTTTCTGGACTGCTCAGCTCCAGTCCTTGGGGAATCCCTGGACACAACAACAGTTCAGGGAATGCTGTA
GTAACAGAAAGCATGGTCTGTGGGTTTGAATCCTGGCTTCATCTTTTTCTTGCTATGTGAACTTAGGAAAATCCTTCAC
CACGCTCAGTCTACCTCTGTTTGCTCATTTGTAAATGGAGACACTAACGTCACATTGTGCAATGCTGTATGTAAGTGAT
AGAACCTAGTGGGGGAAACCCCCACTCAATTCCCGATTCGGCGTGCACCCAAAAAATCACGAAGGACCATCTTGATGTA
ATTACATGAGGTCATTTAATGACGGAGCTCCGGACCGACATGCATCCCACACAGGAGATAACGGATGTCGGCCACGAGG
CTCGAAAGTTAGGGGTTTTTATGGGGTAAGGGGCCTAGGGGCATGGAATTCAGCATAGTGGCACACTATTGGCTTATTC
AAATATCAGCAGAAAAATTACATGTACGTGCAGGGTGTCAGAGTTCTGTGAGTTGTTGACTCAGTTTAGATAGCCCTGT
TCTGTCCTCACATTCCTCTTTATCTTTAAAGTTAAGCTGTTCCCAGGAATGTTTTAACTCCGGGGCATACCCAGGTTTG
TTTTTCTTTTTTCTCAGCCCCAGGCAGCCTCTAGGCTCACAAACAACCAGCAATTTCTGAGTACTTTATATGACTTACA
GGTCTTGAAATTTCATCTTTCTTTCATAAGAAACACAAGTAGAAAGCTGGCCATATGTCACCAGTTAGCCAGCCAGTGC
CAGGTGCTTTCCCAGCTGTATGGATCATCCTCCTGTAGGGGGAGGTTCTGTCACTAACGCTATTTCCCCAATTGGCTCT
TGATGTGTCAATAAAGAAAGCCGGGGGCTAATTGCTGGGCAGGCAGTACAGGTGGGACTTCCGGGTCCCAGGAGGAAAA
GGGAGATGCAAAGGAAGGAAAGAGGCTTTTTCTGCCATGCTTTGGAGGAAGAAGGACACAGCAAACATGTAAGGCCTTG
GGGGAAGCTAGGGCCTGTGGCTTCGGTTACAGGCAGGTGGCCAAAGATGTTTGGCAGGGGCTATTTGGAACAAGCCACT
AAGCTTAGGGCAGAAGGAAATATGGAGATAATAAATTGGTAAGGGCATGTATTTCCAGGCAGGAGATATTTGCACCCAG
CAATTGTGCCTAGCAGGCGAGTTAAGGTAACAAAGCTGTCTGTGTGTGTCTTTTATCTTCGGATTCAAGGGTTTTGGAA
GTGGGTTCATAGCAAGACACGCCCCCCCCCAATGGAGCTGAGCCAGCTTCTGTCCTAGGCAAAGGCAGTAGTGATTTTC
TAAAGCTACAGGCAAAATCTTCCCATGTCTTCCCTTGCTGCTTCCTCTTCTTGTCTCTGAACTCCAACCAGTTCTATAT
CTCCATCCTACTTCTCTTCCTTAGTTGATTTCCATAGTAGCCAACATGGCAGCTTATCTTGTGTGAGACTCCCCCCCAC
CTCTATGCCACATGTCCACAGTCAAGACTCTCTTATTTCAATGGTTTTCCTAGGTGTGAAGGTTAATTTTGATTGTCAA
TGTTACATACCTGGAAAGAGGGATTCTCAGTTAGCTACAGTCTCTTAGATTGGCCCGTGGGCATGTCTATGGGTATTTT
CTTAATAGTTGATATAGGAGGGCCCAGCCCTTCGTGGGTGGTACCATCCCTCAGCAGGTTGGCTGAGACTATACAAGAA
AGGCAGCTCAATGCGAGTTATCAAGCAAAACAGTAAGTGGTGCTCCTCCATGGTCTGTTTTAGTTCCTGCCTTCAGGTT
CCTGTCTTGAGTTTCCTGCCTGACTTCCCCAATGATAGATTAAACTTGTAAGTCAGATAAACCCTTTCTGTGCCCAAGT
TTCTTTTGGTTATTGCCCTAGCACAACAAAGAGAAGAAAACTGGAACACCAGGACACTAGGTCATCTTTTTTTTTTTTT
TTTTTTCAGGGATGTGAAGGTGATATATGATAAAACATGGATTTCTTCTTTGAGAAACCGCCAAGAACGGGATATCTAT
TGCAGTAGGCAGAGAAGGCTATAGGTTTTCCTTCAGATCATTTGGGTTAGGCATCTGATACTCCATAACATCAGCAACT
GTTGGCTTTCAATACAGTCAGAAAGTGTCTAGATGTTTGGCTCCTTAACTTTCAGCAAGCCCAGATGGTGCCCAGCCTA
TCCAGGTTAAGAAGACAGACTCAGATCAACCCACAAGTCAGTTAGAGAACCCACCATTCTGCTTTTGTTACACTTGGCT
CAGCCACTGAACCAACAACTCCTTTACTTAATGCACCAGAAGAAGAGAGAAGAGATTCATTCTCTTGGAAATTGACCGG
GCCCAAGAGATCCTGTGTGTTTCCTCAAGGCTGCAAATCTTACACTCCTAGAGAGCTGCAGAACAAGTGGGCACCAGAT
ATAACCAGAGTGAGGACTCGTAGGTCCAGGGCCCCCTGCTGAGGGAGAAAGAGCATGAGCTAAGCTGGGTGCACAGGCA
CACGGCCATCAGTGCAGCTCTTGGGAACCAGGCACTGAGTCACAGGGGGTAGGCACTGGTGCCCATACCTGTTGGTTGA
AGAGTATTCTTCGAACAAAGTCAGCCTTATCTTTCCTGGAGAACACAGCACGGTTTGGCACTCTGGCCAGGTGGCAGCT
CTGAGCCTCAGTCACAGGCTTCCTGGTGCCATCAAGGCAGAGCAGCTCAAAGTCTTCGTGTTTCAGACCCTTAGCCCAA
GGCTCAGGGTTCTTTCCTAGGGAGAGAATGATGTTGGGGCCAGTGAGGTGGCTCAGTGGGGAAAGAGAGAAAGAGAGAG
AGAGAGAGAGAGAGAGAGAGAGAGAGAGAGAGAGAAAGAAAGAAAGAAAGAAAGAAAGAAAGAAAGAGAGAAAGAAAGA
GAGAGAGAGAGAAAGAAAGAAAGAAAGAAAGAAAGAAAGAAAGAAAGAAAGAAAGAAAGAAAGAAGGCAGTTGCTGCCA
AGCCAGAGGACCTGAGTTGGACCCTTGGGACCCACATAATAGATAGAGGGAGAGAACTGACTCAAGTAAGTTTTCCTCT
GACCTCATAACAAACAGACATTAAGTAAATACGTGTAAGTTTAAAATATAGATATAATTGTTTAGAGACATGGCTCAGA
GGTTAAGGACCAGAGGACCTGGGCTTGTGTCATAGCACCCACATGGTGACTCACAAGTGTCTGCAACTCCAACCCCAGG
GGATCTGATGCTCTCTTTTGGCCTCTACAGGCAACAGGCATGCATGTGGTATACAGACATACATCCTGGTAAAAGACTC
ATAGCTATAAAATTCAAAATCAATTAATCAATAAACAAATCAAATGCCAGGGCTGCAGCTCACTGTAGAGCATTTGCCT
AGCAAGACCCTTGGTTAAATTCTCCAGAACTGAATAAGAATTTAAAATAACAGTCCTGGGAGTGTTAACCAAAGGGGAA
AATTCAACACATTGGAGTGTTTTAAAATTTAAGACTGTTGTCAGACAGTGATGGGTACGCTTTTAATTCCAGCACTTGG
GAGGCAAAGGCAGGTAGATCTCAGAGTTCAAAGCCAGCCTAGTCTACAAAGTGAGTTACAAGACAACCAGGGCTACATA
GAGAAACTCTGTCTCAAACAAACAAACAAACAAACAACCCAAAAAACAAAAAACAACAATTAAAAAACAAACAAACAAA
GGAAGAGGCTGGAGAGATGGCTCAGCAGTTAAGAACACTATCTGATCTTTCAGAGGTCCTAAGTTCAGTTCCCAGCAAC
CACATGGTGGCCCACAACCATCTATAATATGACCTGATACCTTCTTCTGGTATTCAAGTGTACATGCAGATAGAGCACT
CATGTACATAAAATAAGTAAATAAATCTTTTTTTAAAAGTTATATAAAAAACTCAAAAACTCATTAATATTATCCACCT
GGGGTCAAAAGATCTGGCTCAGGGGTTAAGACCTCACTGTTCTTCCAAGGACCCAAGTTCAGTTCCTAGCCAACTTCAG
GCAGCTTCAGGGTCAGTTGGTCTAATGCCCTCTGGCTTTGGGCTCTTGCACACATGTGGTGTGCATAAATCCTCATGAC
CACTCACATACAAATAAATATATAAATAAGTAGACAATTTTTTTAAAGAAAGTGAAAAAAGTCAAGCTACAGAAACAGA
CAAGACATTTTCCAACACGGGATGGCTGTCAAGAACTTTAATCTGGAATACCAAAGGAATGCCTGTCAATCAGGGAGTC
AGACCTACTCACACAGCCCATGGTTTCTGTGGGCTTTGTCAGCCAGGTTCAAGGCCAGGGTGATAACTAGCATCAAGTC
TGAACAGACTGGAAGACCGTTTCAATGCTAGATTAATTTAAAGTGCTACGATCCTAGTCACTGGAGTCTCCTGGGCTTA
GCTATTCAACTTCCCCAGTTGCCTCTTGGGGAGCCACTCTGCAAGGGAGACTCGAGTGTTCCCATGGACAGAAAGCACA
TTGCAGGCGCCTACGTACCATCAGTGTTCTGTAGAACTGTAGGGTGCTTCATGAAGGCCACGTCCCCCTTCTCAACCAG
ACACCTGCACAACAAGGCCCAGGCCAGCAAGGAGAATCAGCAAGGAGTAAGAGGGAGCCACCAAGGGGGAGTCACACCA
CTAGGCACCCAGCACCCCACACAAGCAACTGTAGTGCCTGCAGGGCCCCAGGAAGGAAGGTGATGGAGAGGCAGAACTG
CTCATTGAGTTTCCCTTGTGGAAAGCACCGTTCAGGCCTTGTTTTAAATCCTCACCACTTTCCCCAGACAGGTTGCTTG
TGTACTCCCACTAACAGAGATATCTATGGGATGGCACTCTAGTTAGATCTACAAGACCCACGTGCTGTAGTATCACACC
ATGAGATACTACAATCCGCGTGGAAATATGGCTGTGAGGTGATTTTATTGCACTCAATTGCTCAGCTCTGACAGCACTC
ATCTGCTTCTCTAAACACGACACAATCTTTATCCCATGGGAAAATGTGTACCTGTGGCGACATCTATAACTCCAGCATT
TGAGAGGCCGAGACCAGAGAATCACAAATTTCAGGCCAGTCTGGGCTACAGAGTGAGAGTCTGTCTTTACAAAAACAAA
ACAAAAACAAAAACAAACAAACAAACAAACACTCACTCACCAAACTTGACAATCTGAGTTTGGTCTCTGGATTACAGAT
GGTAGAAAAAGAGAATTAACTGCCTCAAGTTGTTTTCTGACCTCCACATGTGCACTGCTGTGCACAAGTCCTCCCCTTT
CCAACATAGATAGATAGATAGATAGATAGATAGATAGATAGATAGATAGATAGATAGTCAGACAGATAGATGATAGATG
GACAGATAGATAGACAGACAGACAGATATAAATATCCTGGAGCATATGCACCTTACACATACAAAATAATAAGTAAATA
AAATAATAAGCAAATAAAAATGATTTGAAAAAGTATTAAAGTTGGGTCAAGCTTAATACCAGTTACCTTGTGATATAAA
GATAATGTAAGTTAGTTTGGCTTAGATTCAACATTCAGCAGATAGATATTGATGTCACTAGCACATGACTGATAAGAGG
CAAATGTTTGGAGGGCAGGATGAAGTCAACTGATGTATACGGGGGCAGGGGAGTTTTCTAGAGTGCCCCATGCTGCCCA
CAGAGCTTTTACTCATCCAGTACAAACAAAGACAGACAGACATACACACACACACAGACACACACAGGCACACACACAC
AGACACACACAGACACACACACACAGGCACACACACACAGGCACACACACAGGCACACAGACACACAGACACACACACA
CCAATCTGAACATATCCAGCATGGCTGGCTCATCATATTTAGAACTCAGTTTTCCTGTTCAGCCAGGACTCCAGGGTTC
AATAGACATGGCCACCACAGGCAGAGGCAGCAGGTCACAACCCAGACACCTTCCTTCTCCACACTTCAGTCCACAGAAT
TTTATTGCTGGCCTCACTCAGTAGGGTCTTTGCCACACATGCCTCACCCGTGCTGATTCACTTCTCGATTACTCTTGCT
CCAGATTCTCTCTGGCCAATTTCATCTCTCATTATTTAGTTCTCAGCTCTCCTGATCTCTCTGCTCTCAAAGACCTTCC
CCATGTACCTTTTCTTAAGCTGTTGCCTGCCCTGGCCATTGCCACATTACTCCCTGCTGTGCTAGGATCTTGAACATCT
TCCAAAGGCACTTGACTATCAAAGGCTTGGCTCTGGGACCTGGAGAGATGGTTCAGTGTTTGAGAGCACTTGCTGCTCT
TCCAGAGAAGCAGAGTTTGCTTCCCAACCCCCATGTTGGGCAGCTGACAATCACCCCTAAGTCCAACTCCAGGGGTTGG
ACTGGTGTCTGATGCCCTCTTCTGGCATCTGCAGGCACCTGTATGAATATGATCATATTTAAACAGACACACACACAGG
GACACATGTACACACATAAAAATTAAAATCAAATTTAAACTTAAATTTTAAAAAAAGAGCTTATTTCTAAGGTGGTGGT
AATTAGAGGTGAAACCAGAAGGAGATGGGTCCTACTGAGTGATCCCTGAGTCGTTGAGACACGCCTTTGAAACAGACGG
CCGGATCCCAGTTGCATTGGTTCTACTTAGAACCACTAAGATCATGAGCTAAAATGAACCTTTTTGTCTTATAAGCTGA
TTGTCTCGGGTGTTTTGTTACAAGGATGGAAAGCTCCCTAACTCGTGCCTCCTCTTGGTCTCTCTATAGCATATTCAGT
TGAGAACAATTTTCTTTACTTGTCTGTTTACGGTCCTATTTTGTACTGGGACACAGAGCAGACAGGACCGTGCGAGTCT
TGTTGCTTGCTAGCTACCTAGACGAGCACCTGCTACTCAGTGCTCTGTAACTGCATCTTCTTTCATTTAAATGAAAGGC
AGTACCTTGACAGGTCTATTGTAAAAGATCTGTCGGTGTAAAAGAGCTCAGTGGGGGAAGCCAGAGTCTGGGTCTCTCC
TGTGATTGACAGCTGAGCTCTAACAGAACCAATGGCTTATCTACAGTCACACAAAAGATAAGAGGAAAACTAGACTCTG
TACTACTGATCTTGCCCTATCCAAGTCCCTGAAGTGCCCAGGCACCTGGGCCAAGGGAGTATTCTTTATAGAACCAGGC
TGAATATCAGCAGTTAACTCTGGTCTCCAATCCCAGATCCTCCCAAATCACCACCCAGATACTGATATGATGAATGCAT
GAGGAGATGAGGGGGTCAACAGGGTGATGCCAAGAGAAGGAAAGGCACAGACACTAACCTGAGAGCTCCGCTGGAGCCG
TGATACCCCTCAGCGTTGTTGGCAGCACACATGTGGGCTGGGTTGTTACCCCCAACACACAGAGCGCAGAGAGGAGAGT
CAGGGTCAGACCCTGGGGCACAGCTGCGACTGAAGAATGCATCTGCAAGGAAGGACAAGCCAGAGTCAGCTCTTCCCGT
GATCACAGGAAAGCCCCGGAGGTTTGTCCCTGAGACTGCCTGCAACACAAGCACACAGCTTGTTATTCAGCAACATTAG
TAAGGGCTAGGGAGATGGCTCAGCCAGCAAAGTGCATACCACGCAAGCAAGAGGACCTGAGTTTGGATCCCCAGAACCT
ACGTAAAAGCTGGGTGCTGTGGTGTAAATCTGTAACTCCAGTGCTGAGAAAGGCAGAGACAGGTGGATCCCTGCAGCTC
GCTGACCTGCCAGCTTAGTCAAAATGGTGAGCTCCAGGTTCAGTGAGAGACCCAGTCTCAAAAACTAAGACAGGAGCTA
TAAGTACCAGCACTTAGGAGGCAGGGACAGTTGGATCTCGGAACCATCAGGGCTACACAGAGGGACTCTTACTCAAACA
ATAATAATAATGTGGAGATCAACTGAAGAAGACAACCAGCCTCTATCTCTGGCTTCCATAGATGTGTGCATACATGCAC
CTGCACATATACATATCCATGTCCATATGAACACATACATACTCTACATATACAAAACTCATTTTTTAAAAAAGAACAA
GAACATCATGGAATAAAAGAAAAGATTCCAAGCCAGGCAGTGGTGGTGCACGCCTTTAATCCCAGCACTTGGGAGGCAG
AGGCAGGTGGATTTCTGAGTTCAAGGCCAGCCTGGTCTACAAAGTGAGTTCCAGGACAGCCCTGTCTCAGAAAAAAAGA
AAAAAAGAAAAAAGAAAGAAAGAAAGAGAAAAGAGAAGAGAAGAGAAGAGAAGAGGAAAGAAAAGAGAAGAGAAGAGAA
GAAAAGAAAAGAAAAGAAAAGAAAAGAAAAGAAAAGAAAAGAAAAGAAAAGAAAAGAAAAGAAAAGAAAAGATTCCATA
CCACGTAAAGGGACTGACTGGAGATGAGCCGGCCTGAGAGCTCACAGAAGTCTACCTGTCCTCAAACAGGACCAGGGTG
TTGAAGGGTAGGTAGAGAGTGTAACTCTAACATAGTGGAAAGGGAGTGGGCTGGCTTGTTTGACCTAAACCCATGCGTT
CTGGTCTCTGCTGTCTTGGTTTTCCCTTTAGAAAGCTTGCCATTGTGACTTCAAGTTAGGAAAGCCATGGCTGCCAAGC
CTAGCACTGGGTTCCAGGGAACTACACTGACCATGGGCCCTTCCCTTTTCCACTTACTCTCTGTGGGTACCATTCAGAG
CTCAAGCAACCACACCCATGGATCTGCATGGCCCCCGTTGCTCTGGGAAGAAGGAATATGATTTCCTGAAACTGGAGAT
AGAGGGACCTTCCATAGACCAGGGAGTCAAGAGATGGGGAATGAGTAGGCAGTCCCCAAGGGAATGGGGAGAAGAGAAG
CTTACAGACAGGGGACTGGCACCTGAGAGCAGGACCCAGACATAGCATAACAAGCTTAATGGGGGCCGTTTGAATAGAC
TGTCAAGAGAAGGGAGTAGGGCAGGGGCAAGGTTCAGACCCCAGGGGATGTTACTGTGTAGCTACTGTTGTTTATCTCT
AGAGTAAAGGGAGTCTCTGAAGTGATTTAGACAATGCTGGGCCCAGTGGATTGGCACACACAATCAGAACCACACTTTG
CAGAAGTCTGACTGAGAAGTAGATGGATGTAGGGTTGATGTGCAGCTCGGGTGGCAGGCCACTGCAGTGGGCTAGGAGA
GGGAGCGTGAAGTAAGAAGACGTGGGTGGGAGGAGAAAGAACTGACGTTGCTGGGCAAGTGGGACTCCAGAGTCCTCCA
CGGTGCTAGGCAGTCCTGGAGCATCACCAGGTTAGGATGAGAGAACACCGTTTTGGATAATCGAGATGAGTTTAATATG
CCTTCGAGATCTTAAGTAGATATGGAAATAGTTCATTAGATACATGTTTGGGTTGCATGTGGTCATGCACACCTATAAT
CCCAGCACTTGGGAGGCGGGAAGATCAGAGCTTCAATATCAGTCTCAGCTGCATCGCGAGTCTGAGGTGAAGCCCAACT
TCTTGAGACTGTGTATGTGTGTGTGCTGCAGGGGGGGGGGTGTGTGCGTGTGTGTTGCGGTGTGTGTGTGTGTGTGTGT
GAAGGAGCAATCTGTGCTTGAGAGTTATCGCCCTTTAGACACAATAAAAGCAACAGCAGTCAGCATTTATCCAGTGCAT
ACCGAGTACCTGGTTTTGTATGGGCAGCGTAATGATCCAGCTGCATTTGGATGTGAGTGCTTAGGGATGATGCTTAGAC
GGGAAGAGAACTGCTTTATGAAACCCCGATGTTTCTTAACCAGGTTGGAAAGGATAGACTCCTTTAGGAGAAGGAGAAA
AACAACATCTAGACATCATCACGAAAGCCAGGAATAGAGGGGTGGTGGTGCTGGGAGAGCAGGAGCTGAGCAGCTGGAG
GGCACTGTGGGGGTCTCCATCAGAGTGATGCCCTCGGGATGGATGATGCAAGAAGACAGACAGCAGGAAGTGGAGATGT
GAGAGAAAGGTGTGTGGCTCCTGTAGTGTTTTTAAGATCTGGGAGGAAAGGAAAAAGTGAACTTAGACAGGGGCTGCAG
GAGAATGTTGTAGGAGTTAAGAATCTTAAAGATAGAAGACATCAGTGTATTGACGTGTTGAAAGGAATTGTTGGATAGG
CTAAGAAAAGAAAAATAGTTGAGTGTCTTGGGCTTTGTCGTGAGTGGTACCCAAGTGCAGGGAGAGGGATCTGGGCATG
TAGGAGGCTGAGGAGAGGCAGCAGTCAGGAGGTCCATAGACTCAGCAGCTTCTGCTGATGGATGCTGATGGTTTCAGTT
GTCTTCTGATGGTGTCCACTGACTGGGTCATCTGCCAGAGAAGGTGGCAGGTCGGTGTGGGTTTGAGGTGTGTGGACAC
AGGCACAATTATTGGAAGTGTGAGGGGCAGCTGCACTCGGGAGTGTTGGGGGCCCACTGAGGCCACTGGTCAGAGCTTT
TCCAGAGCAATGACTACTCTGTTGTCCTGGGCTCTTCTCCAGCCTGGTTCCTCTGTTCCAGAGAAGAAGACTGGGATGT
ATCAAGTGATAGAGGCAAGGAATCTGAGGTACCCTTGAGGGTGCAGCTGAAGTGGCTAGGGTGGGAGTCAAGGGAGCAG
GATAAATGTGCAAGGGGAGGCAGCACTCCAGGGCCTTCCTGGTGAAAGGGCTTCAAGTGTGAAAGCTGTCAGTTCAGGG
TATATGTGAAAATAACACACATGTGCACACTTACACACACTACTAAGGCTCCATTTATTCCCAGGACTGTTCATAAGAC
ATAACAGGGCCCTTTCCCTGAGATCTGCCTTCAGGGCTATGTACTCCCATCAAAAGCAGGTAGTGAAAGTAGGAGAAAG
GGGAGGAGTTACCTGGGAAGGGGTGGAGTTCACTTTCTGTCAAGCCCATGACCCAAATATCCTAGGTGGCCAGGACATT
CCTTGCCATCTCCTGAACTGAATATCCCCCCAACTCAGGAACAAGCCCAAGGAAGGCACCGCCTTACCTTCAGAACACA
CTTACCAAATTTGCAAGACCCAGTTTGGTTGTATATTAAACCCATGGGGACGTTCCAGCCTTCAGAAGTGCCAACGGCC
GTGTGGCAGGACTTCTTGCCTTGCAGAGATTTCCAGGTGATGCCAACATCCGATTTCTTAACCACAGCCACAACATAAT
AACCTTGCAGTGGAGACACCGAGACACAAGCTCTTTACTGTTTGGAGACAGAGAGTGGGCAAAAGCAGGATCTGGCACA
CTCCCTTCCCCGGGAACATTTAATGGAGTCTCTGTGTGGAGGAGAATGTGAACTTGGTTTCTCCATCTTCTGTTCAGCT
CCATCCTTTGGCTGGTGTGCTGAAACTACATAGCCATTTGCCTGCAAGTGTGACTTTAGCATGGTTCAGGATCTGTGAC
AATGGGAGTGTGGGACTGGTCCAAGGGGGCCCCTGCTTCTCTGGCAAGACCCCCTCATTTTCTAATCTCGCTGCCTTTA
CTTCTAAGGTAATCCTGTGGCTCTCTGGTGGGAGTGAGCATTCCTGCCTTCTTCCTGACTTATGGGACTATCATTAGTG
CTTCAGGTTTTCCTATCTGAGGAATACACTACTGAGTCGTGCTCATTAGTAAGTCAGAGCTGAAGAGTGGAATTGGTTA
GGTTAGACAGATAAAGTTATAAATCTTCAGAATTAGGAAATACCGTCACTCACCCCCACCTCCATTACACACACACACA
CACACACACACACACACATACACACACACACACACACACCCTGCCCTCCGTGTTCAAATGTGGGAAAAACTGGGGCTCA
GCATGGCAGGTGTAGACTTGTCTCACTAGCTATCTTAGATGACCTTGGGTGCCTCTATATGTACCTACACTTCTCATTA
GTACAGTGATGGCTAAAACTCGTCATGTGGGGTGGGGGAATCTGTTGTGTTCCGAGCATCCTGCTTTGAGTGGGGAGAG
TAGATGAATCTGTAGGTTCCCAACAGTGGTGCTAGTGGCAGAGATAATTCTAGATTCGCTTGTGGAAAAGCAATTTCCA
CTGACTGGACTCTGTAATACAAGTAGCTACACTATCTACTACTGATGATGATAATAATAATAAATTTATTATTATTATT
ATTATTATAAAATACTAATACTGTCTTCAGGTCTAAGATTTTTATTCTTATTTCATTAAATGCTATCATCTATCTTTGA
CTTTGTGTGAGAAAGACAGAAATAAAAGATGCAGAGATAGAAAAGAAAGAGGGAAACAGATGGGAGAGACAGAGGTGGA
GAGATATACAGGAGAGAAAAAAGAGATAGTAGAGACATATAGAAAGATAAGAGACAGATTTGGTGTGATTGTTTATGTT
GACTGTTGACTTGATATGACTTAGAAGCACCTTGGAGATAGAGCTCTGGGCATGTCAGGGAGAGTTGGTCTTGATTGGG
TTAACTGAGGTAGGAAAGCAGATGCCATTCCCTGGGCTCCATCAAAGGAGAAAGCTAGCCGAGCCCTGACACTCATTTC
TCTCTGCTTTCTGACTGCTGATGCAACAAGACCGGCTTCTGCCATGCTTTCACCACTGAGGGATTGTCTCCCCTACAAC
TGTAACTAAAACAAACCCTTCCTCCATTAGGATGCTTTTTGCTATTGCAACAAGGAAAATAGCTGATACATTTGGCTAA
ATACAGAGCCCTCTCCACCCATACTCAGACTGAACCTTCTCGATGACAGCCATCATGTGTCTGTCTAAATTGTCTTCTC
AACATCCTTGCTGACCTACATTTTGCATGTGACTGCAGCTCCTGGATATTTCAACTTTTCTTGAAGCAGACATGTTGTT
TCTGACCATAAATGATCAGACTGGAATACACGGCTGATGGAAGCCAGCTCTCAGTTTTGAGTAGGAGGACGCGATTCAG
AGAAGATTTCTTAGCCATGAATATAGAGAGTGGGGAGCTTCAGGCTCCTATCACAGGGAGATTAGGGTGGACTGCTGTA
GTTGAGCAGGGACATGAAAGCATGGGGACCTCTGTGGCCTGGTACTCAACAGGGGCAAAGCCCACCTGAGGCTGTGTGT
GCTATACTGAGCCAAACCTTCAACTCATCAGATCTCTGCTGAGCCGAAGTAAGTATCTGGTATTCATCTTGGGGCTCTG
TCATTAAATCCAACAAAGCAGTCTTCTGGCCTCTTCCAGAAGTATTTGACTGGGTAGAATTAGAGAGAAGTCAAGAAAT
CTCATTTCCAATGGATTCCCAAATGATGCTGATGGTCCAGGGAGCTTACCCATGGCCACATTCCACCTGATTTCAAGGC
TGGATCCTTCAGGGGAAATATAAACATCAGCCATGGAGAGATGTGGGCAGGAGACTTAACTTCTGAAGGTGTTTGCTCT
AAGGACCAGGTCAATTCCCCTGTTATTAAGTTCCAGCTAACCCCAGGGTGATTCTCATTTTCTTCTTCCTCCTCAGCAG
CCCCTGGGAATCTCCTTCTCTCACAAGGCCTAACCTGCTCCCAGATGACTTCTGCCTCTATGCTGACCTTCGTGCAAGA
GTGGACACGTGACAGAAACACCGTGCTGTGCCAGGAGGACTTGGGGGTCTGCTCACCCCAACTCTTCTCAAACTTAACT
GCAGCCACCCCTGGCTCTTTCCCCCTGGGAAGACACCCAGTCCCTGAGCCTCAGTTTCCCCAACTACATCCCAGTGGAG
CAATAATGCTCATTCTACCAGGTTGCTGAGGAGAACAGACAGCAGTCTCAGAGTTCACAGGAGTCCTTTCAATTGTCAA
GTGCCCTGCAGACATCAATTATTATGGCTGCTTTAGGGCAGTTGTGTATTTCAACAGTGAACCCTGGCATCTCTTTTCA
ACTCTATTAAGTTTGGTTTTATTTGTTATTATTTTTTTAATGTATGTGTATGTGTGTGTGCCTGCATGAGTGCATGTGT
ACCACATTCATAAGGGAACGACAGAGGCCAGCAGAGGGCAGCAAAGAGGAAACTGGAGTTACAGGTATTTGTGAGCCGC
CTGATGTGGGTACTGGGCATCAAATCTGGGTCATCTTCGAGACCAATCATCATACTTCACTGCTAAGCCATTTCTCCAG
CCCCGATTTTGGTCATTTTTTAAGGCTTCCTTTGCCTTCTTCCCAAGGATAAGGTTCTGCCTTTGAACCCCAAAGTTCA
AAAAGCTGACTCACTCACCTTCCAAAGGTGCGTTCACACACTTAGACCCCAGCCTCCCACTGCTGTGGGTAGACACTAA
AAAGAAAAATGGAAACACTGAAGGTCCACGTTAAACTCTCACACGAGTTATTCTACCCGAGCTGTAAGCTTCTCTACCT
CAGCTGAGATGGAATAAAATGAGGAATGTTGGCTTAGCAAGCCGTCGCTGGTTATCACCAGTCAAAGGTTCTGCAGCCC
ATTTAGTTTCTCCCCCTAGTCTTTCCTATTATAACATCAGGTTCTACCCCCAGAATCTCAAAACTTAAACAGATCCAAA
GTACAATAAGATGCCCATAGAAGGGGAGCAGAAGAGAGATGGCTGCACATCTTGGCAGAGATGCTTTTGAAGCAGCTGT
CTGGATCTGACATCTTCTCTGAGCTACAACAAGTCTAACCCAGGGGCTGTTTGGCAAGCACTACCCACTTCATGCCCTC
CAGTATCATTCTAGCCAGAATAAGAGATATGATGTTAAACTCTCTCTGCCTGTAACTGTTCCAGCAGGGGCCAAAGATA
CTGTAGAGACAGATACCTGTTGCCCTCACTTTGGCTCTCAGACTATTGGAACAGTAGTCCCCAAGGCACTGAAATGCCT
TCAGGAATCTCATGCTGGAGAAGGGAATGACTCAAGAGTCACTGCAAGTCTCTGAGGAAGAACTTGTCTCCCAGGGAGA
GGTTTCATACAGTTCTACCACCCTGTCAGTCCACCTTGTCAGTCCACCTTGTAGGTGAGACTAAAGCCAAATAGCATGA
TGGATCCATGATGTGGGAGACCACATCATGACAAGGATTGATCATCCATGCATGTCTCTTGCCCTCTTATTTAAACCTG
AACCTTCATATCAGAATCCTGAAGATGGACTTAGGGGTTGCAGCTCTAATGGGTCCGCTTCCTTAAGTGGCTATGGTAG
ATAAGATCAATCAATCTCTCTCTCTCTCTCTCTCTCTCTCTCTCTCTCTCTCCTCTTTAATTGTTATCTGAGGGAGTGG
CTGAGCCTAGCCTGTCATGGCTGCTGAGACCCGGGAGCTAGCAGCAAGTCTAACCCAAGGGCTAGTTGACAAGCATTGT
GGTGGCTTTACCCACCACATGCCCTCCAGTATCATTCTAGATAAAACAGGAGGTATGACGTTGAACTCTCCTTGCTTGT
AACTGTCCCAGCAGGGGCAAAGACACTGGAGAGACAGACACTTGTTGCCCTCACTTTGGCTGCTTGATTCTAAGACCTA
TGGAAAGACGCCTAACACCCACATCTGCAGCTTATCTGCTTCTGACTGGGGAGTCCAAGATTCCACTTCAAAGAGCCCT
AAGCTGCAAAAATAGTCATCCTCATCTTACCAGAAAATGACATTACTTCTTCACCTTTCTGTGCCCATGGCCCAGGGAG
AAATGAAGAGAAGCGCCCCACTCACAGTAGTTCTCTGCCAGGACAGGTACCAGGCCGCAGTGGCCTGCGACGTAGGCAA
ACCCTCCATCCAGGCTCATGGCGTCCGCTTCTCCTTTCTGAAGTTAAGAAAGAAATCAGTCGTAAACTGTGTGGCTCTC
ATTCCTCTGACCCTCAGAGACTTCATCTCATGCACATTCTGTCATGGGTCCTTATTTAACCAGACAGCAGAACTGGTGG
AAATACGGAGTTTAGAAAACAAGACCTGCCTCTGGCCTGTCAGCTAGAAACATGCCCGATTCAAACCATGCTCTCTGGA
CATCCCTGTGTCCTCCTAGAGGACCCTCTGACATGCCCAGAACACACTTTCTTTATGACCATTGTCAGTAAATTCAGAA
CCCCCATGGTCAGATTCAGGTACTATATTTAATCTGTCTCACGGGAAAATGTTTTCTTACAACTAATGGAATCAAATCC
ATTGCCTAAATCCCATCAGCACCACACTCATAACTGCGGTGCAAGAAACGTTTTCAATAAATAACTAACAAAATAATGA
ATAACCATTAAGAAAGTCATTCATAATTAACTTGATATTTTAATCAAGATTCTAAATAGAGCAACTAATGTATTCTTAT
AGTTGTAAGTATGATCCCTGAGATAAAATAGTTATATGCAATGTAGCCCAGATGAAGGAGCGGGGTTTCCTACCATGGT
GGCAGCGATGCAGTCCTCAGGGGTCTCCTCCGTGGCACACGCCAAAGCACCACCGCTCACAGCACTCCACTGGTCACAT
TTGGTCCTCTCCTGCTGGCCCACTGCACACCACTTCACCCTCTGGGAGTCTTCCAGACCTAGGATGCAGAAAGATGATC
CAAGGACCCAAAGAAAGCCCCAGATGGCTGCAGACATGATGCCAGGCAGGGCATCACTGATGGCAAAGAGCGGGTGCGA
CTCATTCCTTGCCATAGCTCCTTGCCATCAGTCATTGTCTAGGAGAAGTAGAGACCTAGGCATTCATAAAGCAGCTGAG
GTGGAGTTTTCCCCTCCCTCCTGATATCACCTCCCCAGGGAAGCGTCTTAGCTCTCCCTTCCAGAACTTCCATCCAGGG
AGCAGCCCATGGCAGTCATGTGCTCTGCTTGGATGGTCTCATCGCTGTGGCCCATCTGCTGTGTCAGCTCCCATCTAAC
ATCTAACACTTTCTTTTGTAGATGCTCAGTGACTATTGTTTGACTAAATTGGGGGGCAGGGGGGCTGGGGGTTGAAAAC
AGAGAAGTGAAGAGAGGGGCGGGCAGCAGAGGCCAGTGGAAGCGGGGGTTGGGGGTGCGAGGGTGGCGCTGCACTCAAA
GCCTCCGCAATGCCTGTTCTAGCTCTGCTCCTGACTGTGATCATGGATGTGTTTCCTGGTCAGTTGAGGATGAATTGTC
CTCTCTGTTGAGCAAACCCACCACTGGGAGAATAGACTCAGTTAACACAAGAACCTGGAACCACCGTGGCTTTAAATTG
TCTATGGTAGGGAGCTGGAGATGGCTCAGTGGTTAAGAGCACTGGCTGCTCTTCCAGAGGTCCTGAGTTCAGTTCCCAG
AAACCACATGGTGGCTCACAACCATCTGCAGTGGGATCTGAGGCCCTCTTCTGGTGTGTACTCATATACTTAAACAAAA
CCTTTAAAGAAAATCTCTATAATAGAAAGATTTTCAAATCCTTTATGTAGGGACCAGAATACCCCAGTAATCAAGCCCC
TTCGTGACATCTGTAAGGCTCTGAGTTCAAGCCCAGCACCACCACTTCCACCAATATAGTGTTCTCGTCCTTAAATAGC
GTAGCATAGCCCTTTCTCCTGGCTTATCAGAACATCTGCTCGTTAGCCCTGGGTATTGAGTAGCACTTGTGGGCTTTTC
TGACCATTAAGGTTAGGTGAAACTTGCAGCCACCAGCAAAATCTCAATCCAAGGCCTCTGCCACACCCGGTGGCTGAAG
CTTGGCAGGTCCCTCCCGAGTCTGACATACCTCTTTTAAGATTCCGAAAGGCAGAAAGAAACTCATATCCCAAGTACAG
ACTGATGTCTATCTTTCGAGGGACCCTTAGGAGCCCATGAGCAGCATCAGTAAACAGCAGGTCCTCCCCGTGAGGGGAG
CCAAAGAGCTGGAAGGGTGATGACTTGTCTTTTCCAAAATGTTCCTGTCCAGAGGCAGGAAAAGAAACAGAGGATCATT
GTTGACACGGAGGTGAAAGTAAAGACAAAAAGAAACCAAACTAGGTAAGGGAGGGGGGTGGGAAGCCATGCGGCCATCC
CTGTCTGTAGTGAGGAATTTGGTTTCTTTAAAATCTCACTTAGGTTATGTAGGTAGACATGTTGTTGTTTTAATCCCAG
GTGTGAGATAAGGGGCTGCTTCAGATTATTCAAAGCAGCTGACCTCCTGCCTCATGCTCTGGCAGGGATGTAATTATGC
TGGCTGCAGATAGTTTAATTCTGAGGACTCGGGAGAGGGTAGAAATGCAGCCCCAAGAGAGGCCAGGAGGGACTCAGAA
GAGGAGGATGGGGGGGGGAGGAGGAGGAGGAGGAGGAGGAGGAGTGGTGGAGGACATGGAGGGGGAGAGGAGGAGGAGG
GAGGAGGAGGAAGAGCAGGAGGAGGAAGAAGAGCAGGAGGAGGAGGAAGAGCAAGAGGAGGAGGAAGAGGAGTAGGAGC
AGAAGAAAGAAGGTGGCAGCTGTAGCCTTTCCATGCTGCTCCTGGTTGCTGTTGTTGGGGTTAGTCAAGTGGTCATACG
CAAAGAGTAAAGAGACAAGAAGAAGTTGAAGATATCCTCACTATGAAGATCAGACTCCCCCACCACCTCCAAGGAACCC
AACCCCAGTCAGCAGAAAGTATTCTAAAGAGAAACCAACGCCTCCTGTCCTCTCTAACCTTCTTTCTCTCCTCCCTGGT
GTTGGGCTGTTGGAAGGAATAAGGGTTGAAAGGGAGGTAGAGGTAAAAGAACCCAAATAAAATAAATAAAACAAATATG
CCTACAAGTTGTGCCCAGATGTGAGCCTTGAAATAAATTAGATAAATAATAATAAAAATAATAATGATAATAATAATTA
ATAATAACACTAACACGTATCCACAGATTCTTTCTGCGATTTCAGTTGCCAACAGTTAACAGCATTCTGAAAATACTAA
GGGGAAAAATTCCAGACAGAGGCATTAAATTTTAAATAACTTTAATTGTAGTATATGGATGTATTTTGATATTATTGAT
AATCCCTTATTGTGCTTAATTTATACATTAATCTTCATCATACATTTGAATGCACAGATTGCTGACTTACAATGGTTTG
ATTTGATTTTTTTTTTAAGCACAGTGACAGGTTTACTAGAGTACAGCTCAGTGAATCAAGGAGTGTCTACCTCCCTATC
TATTATTTATTTACACATCTAACTGGCAAGCTATCCATCTGTATATTTATCTATCTGTAGGGCTTGGGACTTTTTTACA
ATCACAGACATCCACTAAAGCTTTTTAAATAACCCCTTACACACAGATAAAAGGAGATAGCCACACACAAAGACAGTTC
CAGGGCTTCTAACAAGTTCTTTGAAGGACAGTTTTACTCAAATCCCATCTAGCCTGTGCCGTATAAACTTGGCAAACTG
CTAACTCCCGACCTGTAGAAAGGGGGGTCTGAGTTGGTCATGTGCCAGGCAGCTGCCCGGCTCTAGTAGGCTCTAGTAG
GGCTCTTGCTGTGTGTTCACATGAGTGATGACTTTTTCCTCCCTGACTGGTTGCCCTGGTGATGCTAGACGTTCCCTGA
AGTTGCCAGCAGGGATGCAGGGTAGGTGTCCTGGACCTTCTCTTTACCTCAGGCTTGTTGCTAAGGACCAGTTATTAAA
CAGTCTCTTTTCCTTTCAGGCTTTCAAAATACCCCAAATGCCCGTTGCCTGTGTCTCCGTTGCCTGGCTCACAGTTGTT
CCTGATTTCAGCTTTTCGTGGTACCTGTCCAAGTGCTCTTTCAAAGGACTGTGTTTGCTAGGTATGGCAACACATGTCT
GTATTCCCAGTACTTGGGAGCGTAGGGAAGGAGGATAAGGAGTGTGCAAGTCCACCTTTGGCTACTTAGCATAGTCAAG
ACCAGTCTAGGCTACATGAGACCTGTCTCCACAAAAACCTAAGCACAACAACAATAACAATAGCAAAAAGGTTCAAATG
ACTGTAGTTTGCTCTTGCAGTGGCTCTGCATAAAATCAGCCACCTGCTGATTTTTCTAATAATCCTTCACCCCTCTTTT
GGACGGATGGGGACACACACACACACACACACACACACACACACTCCTTATCCCCAGGCCTCAGGATTTATCCCAGAAT
GCCCTTCCAATCAGTTCCTCTGTGAAAGCACAGAGTACAAGGCCCATGTTCTCAAGGTGGTGCAATTCCTCCTGGTGCT
AATGCATGCATGCTCTCCTTGTGCCTGGGTGCTTCATCCCTGAGCCTGAACACAGTGCTGGCACAGATAGGGTCCAGAG
ACTGTCTGGGAGAGATTTCCACCAATACAGAGATCTTGTTTCTGTTCCTCCACCAGGAGAGTCACAGGCCAGGGTAGGG
GGTCACAAGGGAAGTGCTTTGTTGAAGAGGGTGGGACTCCATAGAATGAAAAAGAGAACAGAAATGAGACAGGAAGTGC
TTAGCCCTGGGTTGGTGATGTAGGTCACATGGTCCTAATGGCTTCTACTGTCTGGAATAAACACATAGCAGGTAGATTG
TTGGTGAATGCATCGCCCATTGTATCCTGAATGCACAAAGACGGCTAGTGTTAGTGTGGTCAGATGGAGGAAACCCACA
CATACCACACACACTGAAGCCAGGAGCTTCGAGCTGAGGAAAGGATGTGGGAATGGGGTACCTGGGCCACTCTGAGAAG
CTCTTGGATGGAGTCCTCCTTGCCATCCACACTTCGAGCCACCACAACATGAGAAGGGACCCGGGCCAGGTAGCACTGT
TCATATTCCTCCACTGGCCTGCGGGTGTTGTCCATGCAGAGCAGCTCGTATTGGTCCCTGTCGGCCTTTGTTGGCATGG
CCTCTGAGGAAGGAAAGTTCGGGTGAGCAGGCTGAGCCTGGAGCTGTGCTGACCTCTCCCCACCACCACAGAGCGAGTG
GCCTGAAGGGGACAGAGCTAGAATCCTTTCTAAATACACTTTCCACAGATGAGAGAGCAGCGGGGCTCACCAGGGGAAT
GACAGAGTTATAGTCCCACTGTGGCTCCCACAGGTAGGGTACCATGCCTGACCCTGGCCCCCTCCAGCTGCCTACATCC
CTGTGGATCTGAACATACAAATGGTGGCAGCAAACACACGCTTAGCAGTGATGACCCACACGGCTGTATGCAAGACTTA
CTGCTGCCCAGGCCTCTTTACCATCAATCCCCCAAGCTAAAGGAGGCTGGACATAGAATGATCACATCATAGGCACGAA
GAAACAGAGGCAGAGAGTTAAGGTAACTTGTCAAGGTCTCAGATATGCTGCCATGCTGGGGCAATTCATGTCCCTCATG
AACGCAGCCATGTTCTCCCCGAGACAGAGCCTGCTTGGGGAAAGTGCTGAGAGAGCCGAGGGTAGGAAAGGATACTGGG
GTCCCAGTCAGGGAACTCTGCAGGTGTGAAGCACAGGTTCCTTGGTTGCTACTATGTTGATTTGGGTGTTGTGGACCCT
GTCCCATTCATAGGTTGAAGTCATAATCCACAGCGTCCCCAAGTGTGACCTCACTTTGAAGTAGTCTTTACAAGTGTGT
GCCATTAAGATATGACATAGTGGAATACAGTGGGCTCCCAATTCAATACAATCAGTGTTCTTGTGGGAGAGGAAACTGA
GCCAGAGACGTGCACACAGGGAGACTGCCATGCAAGCATGACATCAGTGTGACGTCAGACAGTGACGATATTTATGTGC
AAGCCAAGGAAAAGCAGAGACGGCCACCCAGCCAGGAGAACAGGACGGGAGATTCTTCCTCACGTCCTCAGAAGGAGCA
GGCACTGTTGACACCCTCATCTTAAAGTTGCAGCCTTAGCTGGGCAGCGGTGGCGCACGCCTTCAATCCCAGCACAGAC
ACACATAAACACATACACAGATAAACACACACACACATACACACACACACACACATACACACACACATACATAGACATA
CATACACACACACACACACACACACACACACACATATACAGAGAGAGAAAGACACATATACAAATACATACACACACAC
ACACATACACAGAGAGAGAAAGACACATATACAAATACATACACACACATACACAGAGAGAGAAAGACACATATACAAA
TACATACACACAGATACAGTTGTTGGTATTCTGTCTAAACTCTACCCCCACAGTTCCTGGCAGCAGCCAGGTAGACCTG
GCCCTATAAAAGGGACTGCTTGGTCCCTCCTCTCTCTCTCTTACACTTTTCTCTCTCTCTTGCCTCTTGCCTCTTGCCC
CCTCTCTCCCCATTCCCTTCCCCCACCTCCTCTACACATGGTCATGGCTGGCCCCTACGTTTCTACTCTCTCCTCTCTG
CCTTTCTCTGCCTCTGCTACCCTCTTAACTCCCCTCCCCATGCCCTGAATGAACCCTATTCTATACTGCACTGGTGTGT
GTCAGGTCCCTCAGGGGGAAGGGATGCCTCTGCATGGGTCTGCCGGGGGACACCCTTTCCCCACACCCTGCCAGAACAC
ATTCTTATAACTCTTTCTCTTTTTATGATCCCAACAGTTGTCACATACACACATACACACCTCACAAAACCAGAATATG
TCATCTTAACTTGTCTGAAAACGTTGCTTTGCTTGTTTGAGCTAAATTGTCCAAAATGGCTGGTGGAAAGGTTTGCCAT
GTTACTTTTGATTTTAATTTTTAGAATGGGGGGGTTATCAGGGTAAAGCCTTTGTCTGGTATATACAAGGCTCTTGGTT
TAATGTGGGCCACTGCAGAAGCGACTTCTTTCATTTGTTGGTAAATTGCAAATAACCGTCCAAAATTCATGTTCCCTAG
ATTTATAGAGGCATCAGCAGGTTGGCTAAGGCAGCCAGAGAGGGATGGGTGCAGAGCCCTGAAATGGGTGTGGTCACTA
GGAGACGGATGTGTGCTAAGAAGAATAAGTGGGGCCCTAAACAATGTAGAAATGGTGGAAATGCTTTCATAAAATCTCT
TCAATGGAGTTAGCCTCTCAATATCAATAGTCTCTAGAAGTTAAAAGCAAAAAAAAAGCAAACAAACAAACAAAAACCC
TTTAGAGACAAGAGCCAGAGCCTTTGGTCAGGCTGGGGCCCACTTTGCTCTTCGTCCAGCACTTACCAAACACTGTCAG
GTGCTTCACGAAGCTCACATCCGCCGTGCCATCCTGCAGACATCTGCAAACAGGACAGCCAAGGTCAGGCCCTCTGCCT
GGGAACACGGTTCATCTGCCTCTTGCGCAATGCCCCTTACAATCTAGGAAGGCTTTGGTAGAAATGGTGGCGCCCATCT
GCCTTCAGAGAGGCAATGGCCTTATCTGGGGCCTTTGAACATTATACAGCCATGCCGAGACCTGAAGCAGGCAGGAGTC
TGGAAATCATCATGGCAGACAGCTCCAGAGAGGGGTGATGAAGCAGACAGGACCCTCACATTCCTCCTAGGGAGCAGCT
CCAAGCAACTGGTTTGGTTTTGGCTACTTACAAGTCATCAGATAAAACCTACTGGTATGAGCCACCATGACCAGCAATA
TAATATCAATCTACCCACAAAACCTTCAATCTCAAATTTGTCCTGCCTACAGGATGTGCAGAGATAAAGATGGAGCAGA
GAAAGAAGGAATAGTCCAACCTGAGACCCATCCATGGGAGAGAGCCAACCCCTGACACGATTAATGATATACTCTGCTA
TGCTTAACAAAACTGTCCTCTGAGAGGCTTCATCCGGCAGTGGACGGAGGGATGCTGATTGACAGACCCACAGCCAAAC
ATCAGATGGAGTTTGGGAAGTCTTGTGGAAGACTGGAATATAGATGGCACCACAAGAAGACCTACAAAGTCAACTAACC
TGGGTCCCTGGAAGCTCACAGAGACTGAAGAGTATGCAGGGGCTGGACCTAGGACACTCACACATTTGTAGGAGATGTG
CAACTTGGGCTTCATGTGGGTCCCCTAACGAATGAAACAGGGGCTGTCTCTGTCTCTGTCACCTGCCATTGGATCCCCT
TCCCCCTACCTAGTGTTGGGCCTCAGTGGGAGAGGATATGCTTAGTCCTGTTAGGACTAGATATTCTAGGGTGGGGCTT
CCCTTCCCCTCCTCTGAGGATAAAGGGAGGGGTAATTGGGTGAGGGATTTGTAAGGGTGGGACTGGGAGGACAGGAGGG
GGGCTATGATTGGGATGTAAAGTGAATAAAAATAAATAAATTAGGAAAAGAAAAAATAAAGGCATCAGATGCTCTCAGT
GACAGGGAGACCTCTTTTGCAGAAAATAGCAGACAGAAAATAACCCTTCCCCAAGCCTCTGAATGCAGTGTTTGAACAG
CAACAACAACACGACTTTGCTTCCAACACTTGAGAGGCAGAGGCAGGGGATCTTTGTGAGCTTGAGGCCAGCCTAGTCC
ATAAAGGGAGTTACAGGACAGCCAGGACTGTTACACCAAGAAACCCTGTCTCAAAAAACAGTAATGAAAATAAAACAAC
AACAACAACAAAAAACCCCAAACAAACAAAAACCTGTCAGATAACCTCAAAACCCTGCTTTGGTTTGAGTCACCTTCTA
CAGAGAATGAAAAAACACAGGTAAAATGCCAAAGGTGGTTCGCTGGGAGACTCCTGAGGCCTGTGTCTATGGACCTCGG
TCTCCAGAAAAGAGATAAGTCTGACCCCTTGGCACTTCTGTTACCATGGGGCATGTCATGGCCCCAGCTTTGGGGAGAC
AATAAGGTCTCCCACAAAGAAGATAGTTCCTCAGGATGGTTAGAAACAGGGGTAGGCCACGGGAGGGACTCTGCACCCT
GATGCTCATGCCATCTAGATGCCAGGAGTATGTCCTGTTCTACCCTATGCCTTCGTCTCTCTTCGATGGCTGATATTGG
TTGTCAACTAGACTGGATCAGATAACATAGGAGACAAGCACAGGTCGTATCTTCGAGTGTGTTTCTAGGTTAATTAAGG
TGGGAAGACACACCTTGAATGTGGGTGGTGTTACTCTATGGGCTAGAGCTTGGCAATGAATAAAAAGGAGAAAGTGAGC
AGAGACCAGCATCCCCCACCCACTCCTGGTAACCAGTTAGCTCCTGCTCCCAGTGAACACCAGCATCCCCCATTCCTGG
TAACCAGCTAGCTCCTGCCCCTGCCCCTGCTCCCACTGAGCGCCAGCATCCCCCACTCCAGGTAACCTGCTAGCTCCTG
CTCCCGCCACCACACCTTTCCCATCATGAAGGATGGTATCCTCCCAAACAGTGAGCTGAAATAAACTCTCACAAGTCGC
TTCTTGTCAGGCATTTAGTCACAGAAAGAAGAAAAGTAACTAAGAGACTGGAGAGATGACTCAGCAGTCTTCCAGAGGT
CCTGGGGTCAATTCCGAGCATTCGCATGGCAGCCCACAACTGTCTGTAACTCCAGTTCCAGGGATTCAACACCCTCACA
CAGACAGACGTGCAGACAACCACAAATACACATAGTTTGATTATTTATGTAAAGTATTATATAACATATAATATAAATT
CTTTTTAATAATTTATTAATTTTTAGTAAATTAATAATCTGATAATAAATTAATTTCATAAATACTTTTTTTTTTTAAA
AAGAGAAAAGTCACTAAGAGACCCCCTTGTTCCAGAGCAAAGGTTTCACTTACTTTAAGGCTCCCCAGGAGCCAAAGTA
TGGTTCCCGGGAGGAGCAGGCACACTTGTCTGTCCCCTTCCCCGCACACAGTTGGCACAGGCTGGGGAACATCTTTCCA
TCCGCACAGGGCACACAGCTGCTGGAGAAGAACGTGGCTGCCGCTGCTCAACACAGAGACAAGGCCCCAGGGTGTGAGC
TGGCTCAAGGCTCCAGGTTACGCATGTTTGTGGGGGAGTGCCCTATGGGAGAGCCTAAGTCTGTGGACATCTCCCAGGC
CTGCTTCTAAGAAGGGAAAGAATGTGCAGGGAGCCTGCCTTAGGTAGTCGGCAGTGCCCACGTCAGAATGAATGGATGC
ATGGCCCAGGGTCCTGTGTGTTTGAGAATCACACTCAGGTGAGCCCAGGGCTCAAGCCCATGAAAATGCTTGCCGGCTT
GTCTGCTGTAAGGTGTGGGAACTGCTGACCCACGTGAAGACAAGACGTGACCCTGTTAATTCACCAACCCCTTACAGGG
CCAGACAGAACACTGAAGACCCTGTGCAGATCGTCCACCACCCAGTAGCCTTGTCTCCCTGCATGAAGCTCACCCACTT
CATCCCTTCTGTGCACAGAGACTATGATGCCTCATGGGATCCCTTTGGTGGTGGGGCATGGGGGTATTTGGTAGATGCT
GCTGGTGAGTCAGGAGAGGAGCAAGCCAGTAGCCTAGCTGCCCAGGGAGACGTCCACGGGTATCTGCCACCAAGGCAAG
GCTGTTCCACTTCACCAGACTCCAGCAGACCCACAGAGTCACCTGTCCCTCTCTGTTAAAACACGAGTACTGGGGGCTA
GAGAGGTGGCTCAGTGGTTAAGGGCACTCACTGCCTGCTCTCCCAGAGGTCCTGCGTTCAGTTCCCAGCAACCACATGG
AGGCTCACAACCATCTGTAATAGGATCTGATGACCTCTTCTGGCATGCACATGTACATGCAGATACAGTGTACTCATAG
ACATAAAAATACACAAATCTTTAAAACAAACAAACAAACAAAAACCCCCAGAATATCCATGAATACTACATTGTCAGCT
CAGAAAGGGTACCACTTTTGCAGAGTGTAGGATCTGGTAATCATCTTCAAGGGTTGAACAGGTCTGGGCTCACTGTGGC
TCATTCTAAATGCTCGAGAAGCCTGGGGTATCCAGAGATGTCCTAGCATCGACCAGTCCCACTGTCCTTGCCCATGCCC
CAAGCCCTCGAATGAATACTTGGAAACCCGAGGGGCTGGTGAAGTTTCTGGGAGTCCCCAGAGACCCTTCCTGGGAGGC
TTGGAGGGAGATGACCTTCCTTGGGGACTTTCAAGGCTACCCTTGCCAAGACAAACTGCTGCAATTCCCAGGGAAGAAG
AGACTGACGGCCCAGCAAAAGAGAAGCACCTCTATTGAGAGATGACTCTGCCAGCTATGTGATGCTAGTGCATACTCTT
ACTCCATCTTGGAGAACATAAAGCATGACATGTTCGTCAGGGAGTGAGCGATGGTTAACATGCATTATCAACTGGATTT
GGAATCACCATGGAAACACATATCTGGGTGTGTGTCTATGAAGATGTTTCCAGAAAAGTTTAACTAATGAGGGAAGAGC
TACCCTGAACATGGGTGTGTCCCCACTGATGGAATTGGGTCCTGGGTTAAATGAGAGAGAGAGAGAGAGAGAGAGAGAG
AGAGAGAGAGAGAGAGAGAGAGAGAGAGAGAGCGCTCCTGCTATCATGAGTTCCCCACTGAGATGGACTGTTCCCTCAG
ACTTCGGGCCCTTCCTTCCTGAAGGTGCTTTTGTCGGGTATGTTTCACAGCAGCAAGACAAATGACTGACCCATTGTGG
AACCCCAACATAACCCATTTAGACCATCCAGTGCAGGAGCCCCACTTAAGGGCACCCAGCCCCAGGTGTCTCACCTGTT
TCCCGAGAGCCAGAAGGAAGCAGGGTGCTGAGGGGAACGTACCACCCAGCAGACCAGCCCAGGCCAGTATGGCAGGACT
TCTTGCCCCGGAGCTGGTTCAGTTGGAAGCCTGTTCCCTTCTTTGCCATGGCCACAACATAATAATGGGTTTTTGGATC
TGTAGTGGAGCAGAGAGAAAGAGAAGGCAAAAATGAAGTGTGTCATTACAACCAACCTTCCAGGGCATTCTCAATGCGG
ACCATCAGAGGACCCTGGGCCCAGCACCACTCCCTAGAAAGAAGTGTGGGGGGTGGGGTGGGGTGGGGAATGAAAAAAT
GCCTTTTTCTGGCAGGAAGAGAGAACTGATCCTTCCAAGGCAATAAATTAATATCACAGATGCTCTCTGACTTACAAGC
TAGACTTGTGAGGCTTTTACTTTATGGTCAATGCAGATGACAGTAGCATCCTTTGAATTTTCAGTTTGGTCTTTCGAAT
GCATGCTGTGCCCCTCCTCTGAAGCTGGACAGAGATGTTAAACCATGGTGTGTGTAGTTAGCCGTGGGATCACCACGGG
AACAAACACTCATCCGCAGCACTGCGGATAGGCTATTAGCCAGACCAGCTGTACTGAAAGCATTGGGCTTATAATATTT
AGTTTCTGCAAGTCATTGTAAGTCTGCAAAGCCTCCAATGCTTCATTTCAGGACTCGTGGGGGTTCCAGGGACCCCCAA
GCTTTAGTCCCAGTCATTGCACTGTTGCTGTCTCTATGTGGACTTCTCTTCCTTTTGTGGTTCTCTGCTTACAGCTCAG
GCACATAGTAAAGTTGTGAGAAGAAAGACGGAGAGTGTTCGTGGACTACCAGACTGAGCCGGGCTGTGCTTTCACAGGC
TGATAGAAATAATGGACGCGAGGTGAATCTCCCCTCATTCACTCCCCCAGAGCTTCTTTTCATACCACCTGTGCCATAT
AAGGCCTACAGGAAGCAACAAAAAGAAGAGCCTTGTGGATCCTCAGAAATGGTATCTTATTGTCACAACCCTAGAGGTG
TGTGATAGGCTCTAGATAACGTTCCTCTGGGGATGTTAACTGTCCCACAGACTATAGAACCAAGGATATGTCTGTCCAT
CAGGATTCATAAAAGCTACAGCTTCCTGTGGAGAAATGTCCCAAACCCTGCGGGTCAGTGAACAACACTGCCAATCGGA
AGAGCTCTGGGGACTTGGTACCCGTAACGTTGGCATGATGCTTCCTTTACACCATTGTCTTGGCTAGATTTGCAACCAT
GTAGGAGACACATCTCTGAGTGTCGTTAAGGGTTTTCAAAGAGGTTTCACTGAGAAGGGAAGAGTCACCCCTTGAATGT
GGGTAGCACCATTCCTGGGATGAATAGAAATGAGAAGAGAGTGGACCGAGCATCAGTCCCCCTCGCTTTCTCCCTGCTT
CCTGACTACAGATGCAATGTGACCAGCTGCCTCGCGCTCCTTGGCATTGCCCTTCCCACAGTGGTGGACTGTATCCTTT
AAAACCGTGAGCCAAGATAGACCTTCCCTTCCTGAAGTTTCTTTTGTTAGGCAGTTTGTTACGGCAACAAGAAAAGTAA
CCGCCACTCTTGGCAAAGCCCCCCCCCCCCAACCATGGTGGCCATCACGCCACACCAGTGATACTGGGAGAACACACCA
TCTTTTGATCCATAGTATTCTGCCATGATAGGTTTCAGGCTGTGGTGAGGCAGGTCAGCTTCAGCCACCAAAGCTCCGT
CAACAGTCACAGCATCTACTTTATTGGCCTGAAAGACAAGGGACCAGACATGAATGAGACTCATCACCCCTGGCGAAGA
CTCTGCTAGTGTCTTTCCCGGGACTCTGATGCCACAGACTCTTCCATAGCTCAGACTTCCTTCCTCTCATTCTTTTTTG
TTTTCCTAGGTTGTCAATATCCATTATTTTTTTAAAAGATTTATTTATTTATTTATTTCATGTATGTGGGTACACTGTC
ACTGTCTTCAGACACAGCAGAAGAGGGCATTGGATCCCCATTACAGATAATTGTGAGCCACCATGTGGTTGCTGGGAAT
TGAATTCAGGACCTCATAAAATAAAAATAAATACGTACATAAAAGAAAGACAGAAGGACGGAAGAAGATTTGGAGCAAC
ATGAATGGACTTGACATGAGCTCCAGCCCTCTCTGAGGCTCCAGTTCATGGCGGCTTTTCTCCACTTCGCACATCCTCC
CTTCCTGAGTCCTTCCCCTTTAGGGTCCAGCATCAGATGAAAAAGGCAAGTCTTAGACAGTTTAACCTGCTCTGCAACT
GCATAAGGACAACTAGCAAATATAATTTATATTTATAGCTATAAATCCATCTCTGCTTACTCTCCCACTTGTCTGCCTC
TGTCTCTGTCTCTGTGTCTCTCATACACACTCATACACATGCACACACACGTATGCATATTAAACAAAGGCAAATTTCA
TGTTAAGAAAAAAAAAACAAGTTTAGCTGGGTAGTGGTGGTACACACCTTTAATCCCTGCACTCGGGAAGCAAAGGCAG
GCATATAGTTCAAGACCAGCCTTGTTAAAAGAACAAAAGCAACAACAACAACACCCCAGCTTTTTGTACTTAATATTAA
AACTCCACCAGGTATGGTGGTCTACACCTTGAGTCCCAGTGCCTGGGACAGAGGAACGGGGACACTTTGAGTTCTGGAA
AAGCCTGGTCTGCATAGAGAAAGTTTCTTGTTTCATTTTGAGTCTTTTTTTTTTAATTTTTAAAATTTTTATTTCATAT
GCATTGGTGTTTTGCCTGTAAGAGGGTGACAGATCCTCTGGAACTGGAGTTACAGACAGTTTTGAGCTGCCATATGGGT
TCTGGGAATTGAACCCTGGTCCTCCGGGATAACAGCCAGGGCTCTAAACTGCTAAGCCCAGTACTTTTGAGTCTTTATA
GATTCTCTTGATGCAGTTTTAGGTTTTGCTGTGTCATGGCTACATTGCTAAGTTTGAACCCTGTATTAGTTGTTTGATT
AAGATGATGACTTTTCCGACAGACGAGCACTGAACAGCTGTGTTCTCCCCAGCTCTGTAAAATACAGGAAAAGGACAGC
CTTTCTTCAGCTTTCTTTCACACAGCAGTGACTTACCGAGATGTCCCGGATGCACTCAGGGTGAGACATTTTCCTTACA
CAGGTAACAGCTGGGCCACCTGCAGGAAGGACTTTCTTCATATTGTCACGGAAACTAGAACACTTAGTGGCCTCATGAT
CTGACACAACGCACCATCGTATGGTCTTCTCAGGAAGAGCCAGGCACAGCCCTAGGGGAGAGACACAAGAGGTGTCAGG
GACCCACAGCACTTACCTTCCTCTGCTGGCAGAAAGGAATCAAACACAAGACAAGGAGCTAAGTGGCCTGGACCGATGG
CACAGAGAGATGGTGTCACCGTTAAGTCTGCATTCAACTCTTCTGGAGAACCCAAGTTCAGTTCCCAGCCCCTGAGTCA
CAGCTTACAACCTCCTGTAACTCCAGTGCTGAGGAACTGGACATTATCTTCTGGCCTCTGTTGTGGATACCTCAATTAT
ATGCACAACCCCCCCACAAACACACACACTATGTGTGTAATTAAAAATATCTTAATACTAAAAAATAGAGCCAACTGTG
TGTCATCCACACACTGAATTAGCTGTGTTCCTTGAGGGAATTCTCAGGGCCCTTTTTTTCTGACTCTCTCCAGATATAT
TAAAGTCCCCCAAGTCAGGCGTGGTGGCGCAAGCCTTTAATCCCAGCACTTGGGAGGCAGAGGCAGGCGAATTTCTGAG
TTCGAGGTCATCCTGGTCTACAGAGTGAGTTCCAGGACAGCCAGGGCTATACAGAGAAACCCTGTCTCGAAAAACCAAA
ACCCAAACAAACAAACAAACATCCCCCAAAAGATAACCACAGAATTAGCAATATGACACAGCATTTTCACTCCTAAATA
TATATTTAAAAATTGACAACAGGAACTCAAACAAATACACAGATATTCACAGCAAAACTTTCTTTTTTTTTTTTTCAAT
AACTAAAAGAAAAACATTCAGGGCTAAAGGTTAAAAATGCTAGCTGCTGGGCTGGTGAGATGGCTCAGTGGGTTAGAGC
ACCCGACTGCTCTTCCGATGGTCCAGAGTTCAAATCCCAGCAACCACATGGTGGCTCACAACCATCCGTAACAAGATCT
GACTCCCTCTTCTGGAGTGTCTGAAGACAGCTACAGTGTACTTACATATAATAAATAAATAAATCTTTAAAAAAAAAAA
ATGCTAGCTGCTCTTCCAGAAGTCCTGAGTTCAATTCCCAGTAACGACATGGTGGCTCACAACCATCTATATGGGATCT
GATTCCCTTTTCTGGGTTTGTGTGTGTGAAGACAGATCATTCATACACATTAAAGTAAGTAAATAAATAAGTAAACATT
AAAAAAAACCATCAAGTGTGCATTAAAGGGAGAGTAGATAGGTACCATCATGCTTTAAGTCCTGAATGAACCTCAAAAA
CAGCAAGTGAAAGAAACAAACAAAAACCCTCAACATATAGTTACTAATAGGACCCAATAATTTTAGATATTTATACAAA
AGAAACAAAACCACGTATCCTCTCAAAGATGGGCCCACTACTGTGCATGGCAGTTATTCATAAAAGCCTCTCACTAGCC
GGGTGTGGTGGCGCACACCTTTAATCCCAGCACTCGGGAGGCAGAGGCAGGCGGGTTTCTGAGTTCGAGGCCAGCCTGC
TCTACAAAGTGAGTCCCAGGGCAGCCAGGGCTACACAGAGAAACCCTGTCTCGAAAAACCAAAACAAAACAAAACAAAA
GTCCTCAGGGGACATGTGACCAACTAAAGTTGCCTTCAGCACAGTGATACAGGGTTTGGCCATTGAGAGAGTGAATGTG
GTATATGCCACCTGGATAAATCTCCAAACAACTATGCCAAGTGACAGAAGCCGCTGGGACTGTGGAGGTGGCTCAGTAG
ATAAGGAGCTGCCTGGACTCAGGAGGCAGAGACCCGGATCTTGTTTAGCTAGACTAGCAGAGTTGATGAGCTTCTGGCT
CAGTGAGAGACCCGGTTTCAGCCGCTAAAATGGACCCAGTGTCAATTGCCAGCCTTCGCACAAACTCACACAAGTACAG
TGTGTGTGCACACGAGGACACCCACATGTGATTGAATGTATGAATGTAGCACACATAAATGGGGAGAGGGCAGTGGGGA
AGGAGAGGGGAAGGGAGGGAGGGAAGGAGGGAGAGAGAGAGAACTCCCAGTTCTGTGAAGTAGACCCCTTGGAACCATT
ACAGCCCCTCCACCATTGAGGGCTTTATCCCCGCTGTCTACAGCAATTGAGAAGGTTTCAAGGAATGGGTCATTCAAAT
ACCTATGAGTTATTGATGTTTATTGATGTTTATTGTCTATGTATGGAAACTGCCCTTACCTGTGATCTCCCCTGCTTCC
AATGGGGTCCAAGCCAGACTCCTCACATGGCGGTGCACAGCTAGATCTACTGGAATCCCTGCTCAAGACTTCAAGGTTG
TGGCCCTTACCTGCACTCATTATGGATTCTTGATTCTGAGAGCCATAGAAATCATTCTAAAATCCAAGTGTAACTAACA
GCCCTACCTGCTCTCCCACTCAAAGCCCAATTCTTTCCTCCAACAGGCCTCTGTATTGTTGGTGGGAAGGAGGGAGTGG
CTACTTGTGTTACTGACAATTTTTCCCCCCAGAAATTCCAGGTTCACTTTGGTTTGAGTATTGCACACTTGCATCCAAA
TTAATAACTTATTTAATTCCACTTCTTGCTTAATAATTACAATCTAATGCGGGGGTGGCGTGGTGAGAAGGAGGAGAGA
AAGGAAGAGGAGGAGAAGGAGATGGAAGAAGAAGAGGAAAAGAAGAAGGGGAAGAGGAAGAGGGAAAAAAAAGAAGGAG
AAGTCAGTAGTCCAGGTCGTGGAGATGGCAGAGGGTAAGGGTGCTTGTTGCCCAACCTGATGACCTGAGTTTGACCCCA
GACCCCATAAGATGTAAGGAGAAAACCAACTCCAAAAGTCTTCTCCTGCAGAGCACATACACACAGACAGAGCACACAA
TAAAGACATAAATGGAAAACACAGAGAAGCCAAACCTACAGGGATGTGTGCTTACACTCCAGATGTGTGAGCCTAGGAG
GCAGGGTGTTTGCAGGGGTGAGGTGGGGGTGGCCGAGAAAGGAGGAGGGGCAATTTGAAAAAGTTAAAAATGTTCTTTA
TCTCGATTATGGAGGGGGGTCACGTTAGCCGAACTGTATATATCCTTTAAATGAGTGTAGTTTATCGTATGCAAACAAT
GTGTCAATATTCTGGGGGAACACTGCTGAGCCACTTCAAGTCTCAGAACAAAGCGGCTTTGGCCTCAAAGCAGGCTGGG
CTCAGAGCAAGTCACTACTGCCTGTGAACGGGACTACTGCTCTATGACAAAGACTTCATCATAACACGCCCTAGCTAAA
AATGATAGATGGATGTCACGTTAGGACTGGTAGGTTTTACTGAGCCATTTCTAAGTCACAGCAAGATCATCCTAACCTC
AAGGTCAACCTGTTGATAACCATAAGCCTCAACTGCCGCCATTGCTGGAGGACTGCCTACATAGAAACACACACATACA
CAGACACACAGACACACACAGACACACACACAGACACACACACACACTCAAGCAAGTACACATGGAGGACTGCCTACAT
AGAAACACACACAATACACAGACACACAGACACACACAGACACACACACACTCAAGCAAGTACACATGGAGGACTGCCT
ACATAGAAACACACACAATACACAGACACACAGACACACACACACACACACTCAAGCAAGTACACATGGAGGACTGCCT
ACATAGAAACACACACATACACACACACAGACACATACACACACACACACACACACACACACACACACACACACACACA
CACACACACTCAAGCAAGCACACATGCACCCCTCCCCAGTTCTTACAAAGTTTCTGTTTAAACTGTTTCTACTGCCTAC
TGGTTGTGCCACAGTGTAGCTAATTCTCTACTACTGCTTCCTTCTGTCTTTGGTGAATTCATTCCCTGCCTGAGCATCA
CAGCTCTGTCACCACTGTCCTGGCTTGGCAAATAGAAATATTTTAAAAAGAGGACGTGTACAGACAAGCTAAGGGTGGG
TGGAGTCTGATAACATCAACAAGGTCATCAATAGCATGCCCCCACTAGCCTCCTGACAGACACAGGAAGAGGTCTGAGC
ACGCTCTGACCTTTTCAGGAGGAAGAGAAAGGCTGCTGGTGGCCTCCAGTCACTCACTGAATGGAGAAAGTTCCTGGAC
CCCAGAAGAAACCGTTGGCTTGGCTCTTGACTTTTGCAAGTTTTTAGGATATGTAACCCTCAGTTACGTGTTTCCTATT
TAAGCTGCTTTGGCTGGGGTTTTTTGTTGTTGTTGGTAGTGGTGGTTTGGGGTTTTGTTTTGTTTTTTGTCATTTGACA
CCACAAGTATTCTTATTGGACTGGAACACTGAGTTTTGAGGAATGAAGGTTCCAGACTCCGGCCTGGTCTGGTAGCCCG
GAGAAAGGTCGTGAAGAAACGAAGCCCGGAGGGGCCTTTAGTCTCTGACATGTAAACATGAAGCCTGTTGGGAAAATGA
ACTTCTGGTCTGTGTCTGACATTGCTGGCAATGGTGGCGGAACGCTATTTAGCAGAACACTGGCTCAGTTCAGGGGAAT
TCTGAAAGAGATCAGGCAGCTGCATTCTGAGAGCGAATGACCCTCTGGGTGGCTACAGATGGCACTGCTGGGCAGAAGA
GGCAGACATATACTGTGCACCATTGCCGGGCTCTGGCCACCCTAGGTATTATTTACCACGGTCTCAGATCAGACAAAGC
TAAAGGGAGGAGGATCCAACCGTGGTTGAGACAGGAAGAGCTCTTCACAGTGCATGAGTTCTTCCCTGGAGCCTGTTCT
CCCTACCCACGAAAGGGCTTCTTGTATTTGTGAGCGTAGAACTCTGCCTGCCTCTGGCTGTGTTAGCTCATTTCTCCAG
CAACTGTGTGTTTGTTGGGTAAACATAACTTTTTTTAAAAAAAAGTTAACATTGTGTGGAAGCCACATGGGGGTCAGAG
GACAGATTTCTTTTCTTTTTTTTTTTTTTTTTTGGTTTTTTTAAATTTTTATTTTATGTGCTATACTTATTATTACTAC
AGAGGACAGATTTCAAGAGTGGATTTCCTCTTCTACCATTTGGTCCTGGAGGTCATATGTCGTCAATCACAACATCAGC
CCCTTTACCTACCAAAGCAGTGAAACAAGTTGTGGGGTTTTTTTGTTTGTTTATTTGTTTTGTTTTGTTTTTTTTATTT
TCCTCTCCTATCTCTCATATATTGCTGCAATTTCCCCTCCTTCCTCTGAGTCCCTCTTCACATCCCCTGTCACCAACTA
CGTCTCCATTTTCCTTCAGAAAAGGGCAGGCCTCCCAGGGATATCAACCAAACGCGACATAACAAGTTACAAAAAGACT
AGGCACCTCCCTGGTATTAAGGAGGATGTGGCAGGAAAAGGGTCCCAAAAGCAAGCAAAAGAGTCAGCGACAATCCACT
ATGACCGCCTCAGTGAGGGCAAAGGACAGCCGGGGGACTGCGTGGCAGGTGAATTCCAGTCCAAAGTGATTACTCTTCT
CTAGGCTCATAGTCTATCCAACCACATTTGATCCCTGAAGTGTATGGTCAGCAAATGACACCTGGTCCCCTCCCCAGAG
GGCCGGCCCCCTGCCTGGCTCTCAGCACACTCACCTAGGGTCCCCAAGCATAGAAGGGCGCAGATGAGCAGCCTCATTT
TCAACTGTGGAACTGCGTTCTGCTTGCTCTCCCCCAGCCTAGGTGCACTTTACCCAGAAGGCAGTGTTGACTGATGAAT
CTGCCTACTGGGAAAGGTTCTTCAGCCCCAGTCCGGGCTGGTCGCCACTGCCACACCCTCCATGCCTTCCTTATCTGGC
CAAGCAGATACTCAGGCGGCTAGGGACACTATTTTTTCTCTCTCTGTCCCCGGTTCGGTCCCTGGGTGGAAACGGTGCT
TCTCCGGGAAAGCCGGCGCGCACATGCATAAGACCTTAACACAATTTTCTTTTGGAAATTCTCACTTCTCACTAAGGAC
TCTAAGTTGACTAAGCTGAGGTCCTCCCGCTGTCCTGCGACCCCAGTGGAGCTATCTCTTCTGTGACACAGCCCATAGT
TTCTCCTCTATCTCCCCACCCCCACCCCCACCCCCACTCCACACCACACGCACCAGTGTTGCTTGTCCCAGCTGTGTAA
GGCCTGGCTCAGGCACAGATCTGACATGCAGAGTTTCTCGCTCACCCGGCTGCAGCCCTGTCCGAAAGGCTGTGCGACC
TCTGCCTAGAAAAAGTCTGGTAATTTTAAATAAAACTCGAAGAACTTCTAATTAAAATATAGTAGTAGGTGTACAACAA
ACCCTGCTAGCACAGTGTAATAATTATCACTAAGAGGACTCCGGTAGAGCCAGTCCACATCCAGGAGGCCGAATCCCTG
CAGAGCTTCTCCCACATAGGTGGCCCCTACATGGAACAGAAACGGAAGTCTGCTAACTTCAGGACTCCACAGAGATGAA
AACTTACAGCGCAGAGCCTGGCTTCTCTCACCCAAGTTTAAATTGGTATGATTTATCTATGCAGAGGTGTGTGGTTGCC
CACCAGCTATCAGTGTGGCCTATTGTTTCATATCATGTGAATGAAGAATATTTCATCTCTCACCCTGATTGACAATTGT
AGTACCGATTCCATCCTTACAGACAGTGCCGCTAGGAACAGAAATTTCATTTCATTTTATATACATTACTAGGATCTAA
AACTAAATAAAAAAAAGCAGGGCCTGGTATGGAAGTACAGGCCTGAAACCCCAGCATTTGGGAAGTGGGGACAGTGGGA
TTAGGAGTTCGAGGTTTCAACTACGTGCCCTATTGGAGACTTCCCTAGGCTAGAGACCTTGTCTCGAAAACCAAGCATG
TAGGGCTGGAGAGATGGCTCAGCGGTTAAGAGCAAGTGCTGCTCTTCCAGAGGACCAGAGTTCAATTCTGGCACCCATG
TTGGACGGCTCACAACTAACTACCTGTAACTCCAGCGACAGGGGGACCTGACACCCCTGGACTCCAGCTACAGGTCATC
TGGCTTCTGTGGGGGCTCACACTCCTTTGTATATACAAAAAGCAAAAACAAAACTTAAAGAAAGAAATAGAAGACACAA
TCAGGAAAACGGCACAATTTTTGCACATTGAACTTGAGCACAGTGTCTTTCTTGAGTTATTTTGTTAAATCTGTTTCTT
TCCAGTGTTCTGTTTGTGTTCTTACATCTTTTGTTGAATAATGCCAGTTTTTCTTCCCTTTCTTTCTGATTTTTAGCAT
CCCTCCTTATCCCAGGTTTGGCTTACTATAAACAGTTAGTTTGGGAATAGTCTAGTGCATGCCTTTGAGTGCAAATAGA
CAGGCATCTCTGCAGCAGACTTTTCTAGGAGAACAACTGTTTGGTCCAGAAGTAGGTGGAGGCCTGGTTTCATAGATCC
TGGAAACTCTTAGGACCCAGTCAAGAAGCTCTGAAAAGGGTTATTACAGAAAGAAATGTGTACTGTCTTCTGGTTTTAG
GTGTTATTGGCAATACTAAGGTTCTCTCTTTCTACATGAGTTGCTTACATCTTAAAAACATGACTGGGATTGGATTTTT
TTGTATTTATCAAAATAATGATATGATTTTCCTGCCTTGTCTGTGGGGAACTATGCAGCAGAGCATGAGTTTTCTTCTT
TTGTTGTTATTTGCTTGTTTTTGTTTTTGTTTTTTAAATGTTTTGTTTTGTTGCTTTTTGAGACAGGTAGGGTCTTGCT
ATAAACTAGGCTGGCCTCAAATTTGTGATCCTCCTGCCCCAGCTTCCTGAGGCCTGGACATATGGGGGTGTATAACCAC
ATCTGACTTCTAGTTCGCTTTGTTTGCTTGCTTTGTAATGGAGTTTGAACCCCGGGTCTCACATTCGCTGGGTAAGCAT
TTCACCCCTGAACGGCATCTCTAACCTGCTAACTGGATTCTAGATTTATTTCCCTTTATGGATTAGCAGTGCTTTGCTT
ACATGTGTGCCTTCTGCTTGTGACAGTGAGAACAGGGTGTCGAATCCCCTGGAACTGGGGTTGCTACTGGTTGTAAGTC
ACCATGTGGGTACTAAGAACAGAACTCTGGCCCTTTGTAAGAGCAACAAGTGCTCTTAGTCACGGAGTGGTCTCTGCAG
CTCCCTGCTAACTGACTTTTAAGTATTGCCACTGTTAGATTTCTGCAATAAACCCCATTTGGTCATTATATGTAAGATT
ACCCATGTACTGTTGGGTTGGTAATATTTTCTTTAGTGTTCCTTTCTGTATCTATTCTCTTGAGAAAAATAAATCTGGG
GGCTGGAGAGAGGTCTCAGCATGTTCCAAGAACCAGAGTTTGATTTCCAGAACCCGTATATGGTAGCTCACAGCTGCCA
ACTCCAGGAGGATCTGATGCCCTCTTCTTGCCTCCACAGACACCTGCACTCGTGTGTGTGTGTGTGTGTGTGTGTGTGT
GTGTGTGTGTCTGTCTGTCTGTCTGTGTGTCTGTGTGTCTGTGTATATATGTATATATACAAGTGTAGTGGCTATTCCT
GGTTGTCAACTTGACTATATTTGGATGATCTACAATCCAGAATTGGAAGGCTCACCAGTGACCCTTATCTGGAGGCTTG
GAGATAGAAGTTTCTGATCTGGATCTTGGTATGGAGATCTTGAGGCATAGTGGCTATGGATTAAAGGTGGAATACACCT
TTAATCTGGGCTACACTTTCTGCTGGAGACCATATAAGGACATTGGAAGAAGGAATCTTGCTCTCACTCCTTCGCCTTC
TTGCCGTGTGGGACTGAGTAACTGCTAGATCCTTGGACTTCCATTCACGGCTACTACTGAACCATCGTTGGGAATTGGG
CTGCAGACTGTAAGTCATCAATAAATTCCTTTACTATTTAGAGACTATCCATAAGTTCTGTGACTCTAGAGAACCCTGA
CTAATACATGCAAGTATATATACATACATATATGTGGGAATATATGCATACATGATAAAAATAAATACATCCTTTTTAA
AAAGAGAAAAATAAAGAAAAATTTAAAAGAGAGAAATAATCTTACAGTTTCTCTTTATTCTAATTTCCATATATCTTTT
GCTTTAACATTACAATGATTTTTGAACTGAGAAATGTCACCTCTTACTTTCTGAAAAAGGCTATGTAAGAATAACATTC
CTAGCTGGGCATGGTGGTGCACGCCTGTAATCCCAGCACTTGGGAGGCAGAGGCAGGCAGATTTCTGAGTTCGAGGCCA
GCCTGGTCTACAGAGTGAGTTCCAGGACAGCCAGGGCTACACAGAGAAACCCTGTCTCGGGGAAAAAGAAAAATAGAAT
AACATTCCTTTTCCCTTAAATGTGCAGAAGACATCAGCAATGACGTCCCCTGAGTTTAGAGCTTCCCTTGTGGGAAAGT
TTTCAACAACACACTAACCATTGTCTCCTCCCTCCTCTCTCTGGTACTAGGGATTAAACACAGGGCTTCATACATACTA
GGCAAGAACTGTGTGAAAGAGTTCAGCCCTTCACTTTTACTTTAGACTTGATCTCACTATGAAGCCCAAGCTGGCCTAC
GATTCATAATCCTCCTGCCTCCATCCCAGTAGCTAAACTTAGAGATGTGCACACCCAGGAAATTAAGATTTATTTATAT
TGCTGGGCAGTGGTGGCACACACACTCTAGGACTCGGGAGGCAGAGGCAGGCAGATCTCTGAGTTCGAGTCCAGCCTGG
TCTGCAAAGTGAATGCCAGGACAGCCAGGGCTACATAGAGAAACCTTGTTTGAAAACAACAACAACAACAACAAAAACC
CAAAGAACAAAAAACAACCCCTCCCCCCCCCCCTTTTTTTTTTTTTTTTTTTTTTAAGACAGTGTTTCTCTGTGTAGCC
CTGGCTGTCCTGAAACTCACTCTGTATACCAGGCTGGCCTCAAACTCAGAAATCTGCCTGCCTCTGCCTCCCAAGTGCT
GGGATTAAAGGCGTGCACCACCACACCCAGCTACAAACCCATTTTTATATTTATAGAAATATCCATAATTTCTAATATT
TCAAACCAGAGTATAGGCAAATATTGTCTTTTACTTATTTGTATGTGTATGAGTGTTTTGGCTGCCTGTATGTCTATGT
ACCACATGTGTGCCTGTGGAGACCATTGCCCCTGGGACAGGAATTACTAATGGCTGTTAGGATCTGTTTATGGATGCTA
GGAATCAAACCTCAGTCCTCTTAAAGAGCACCAGTGTTCTTAATTTCTAAGCCATCACTTCAGCCAAAGTATTGTAAGA
AACTTATCTAGAGGCTGAGGCGATGGCTCAGTCAGTTAACTGCTCCTTGTGCAAGCACAAGGCCCTGAGTTCAGATTCC
TGACATACTACTTAAAAATCCAGGCAAAGGGGCTGGAAAGGTGGCTCTGAGGTTATGAATACTGGCTACTCCTCCAGAG
GACTGAAGTTTGATTCCCAGCAACCACATGGAAAATTGGAAATGGAGATTTGAAAGCACACAATTTTCTTTAGCTGGGC
CTGGTGGCACACACCTTTAATCCCAGCATTTTGGGAGGCAGATCTCAGAGTCCAAGGACAGCCTGGTTTACAGAATCCC
AAGACAGCCAAAGCTCCATAGAGAAACACTATCTTGAAAAAACAAAGCAACAAAACAACCAGGCATAGTGGAGGCTGCG
AATCCAGAGATGGGGAGGCAGAGATCCCTGGCCAAGCTGAACCAGTGAACCCCTGGTTCAGCGAGAGACACTGCCTCAA
AAAAAAGGTGGAGAGTTTCTGAGAAAGACAGCCAGCCTTGATCTCTGTCCTCCAAGAGTACATGCATCTCACACATCTC
GTGAGCATGTGTACACGCATGCACATACATAAATCACATACACACACACACACACACACACACACACACACACACACGC
ACACACACACACACACCATCTACATTTCAAAGTTACTGAAGCAAAGTCACTCATCACATTATCCTGTAACTGTTATTTG
GTGTTCTGTACTGGCGATTGGATCCAGGGCCTTGCACTTCGTAGGCTCCAGATCTGCCACTGGGTTAGGTCTCTAGTCC
TCCTCCCTTTAAAAAAAGCAAAAACAAAAACAAACAAAGAAAAAAAACCACTTTGTATTCTAACACAGGGTCTCACTAA
ATCTCTCGGGCTGGCCTGGAACTTATGACCTTCAGCCCCTTGAGGTGGCACAAACTGCCACCAGATCTTGTAGATGACA
TTTTAAATGTATGACGGATTCTTCCTATAATACCTTTTTCCATTGTTGACACTGGTAACTGTTGATTACAGTTGCCGAG
GTTTTAGCTATTTTATTAATTTAAGAAAACACACAAACTTGGGTTGGGGAGATGGCTCAGTGGTTAAGAGTATTGACTG
CTCCTGCAGAGGACCCAGGTCTGGTCCCTAGCATTCATTATCAAACAGCTCACAACTGCCTGTAACTCCAGGGCCTGTA
ACTCTAGGAAACCCAATGCTGTCTTCTGTCCTCCACAATCATTTGCTAGCACTCACACAGCATACACAGACTCGAACAG
GCACGGACATACAGAAATCTTTAAAACAATATCCCCTACATACAAACTTTTGGCTTTGTTGATTTTTCTGTATTGTACA
CTCATTTTATTTCATTGATTTCTGCTTTCTTGACTATCTATGGATTTTATCATTTCTCTAGTGGTTTTTGTTTTTTTGT
TTGTTTTGTTTTTTGAGATAGGGTCTTTGCTATGCCCTCAACCTCATCTGGAACTTCCTGATTGCCTGATTTAATTGCT
GTGAAGAAGAAATATTCAATATTTTTTGGGAGATGGGATTTTCGAGACGGTTTCTCTGTGTAGCCCTGGCTATCCTGGA
ACACACTCTGTAGACCAGGCTGGTCTCAAACTCAGAAATCGGTCTGCCTCTGCCTCTCAAATACTGGGATTAAAGGTGT
GCACCACCACTGCCCACTAGAAATATTCAATATTCTAATCTTGGATTGGATCATGGTGTCTGATTATGATGCCTGCTAT
AATATTCAAACATTTATAAAACAGATGAATAAATGCACCAAGACTATGCAGGGCAGTCTCTTCTGCAGGCCTGTAAGCA
AAGCCCACAGGGTCCAGGAGCGACCATTTCTCCTAGAGTTAATGATCTATCTAAAGCTAACACACTCTAAATCCATAAG
GGCCAAAGTACCTACTGACCTGAGTGGAGTATGCTCTGAAAATGTCTCATGTCCAAGGACAAACTCCTATAGCAAAGAA
AGGACATCCAAAGTGCTAGAGTCATCTTGTGGGCTCAGGCAAACAGACACCAGAGAAAAGATTAGAAAGATAGGACACG
GTGACTCCTGTCTGCAGTTGCAGCACTTGGGAGACCAGAGAACTAGGGGTTCAGAGGCAGCCTTAGCTACATAGTAAGT
TTGAGGCTAGCGTGGGCTACATGAGACTCAAACAAAACAAACAAACAAACAAACAAACAAACAAACGCCAAATCAAACC
AAACCAAAATCCAGAAGATTAAAAGAAGAAACAGTTTTCAGGACTTTGAGCCAAGAGTGATTTTTGGAAAACTGCAGAT
TTGGGCTACTCTTTACTAGACACTACACTAAGTGTTTTTGTAGCATTAATTTATGATGAGCTTATAAGTAGTATTTTAC
ATGTTTTTTTTTTTTTGCATTCAAGAAAAAAGGAAAGCAGAGAAGTTAAGAAATATGTCAAAGGTCCAAAGGTTCATGG
TTTTTGCACATGTTTTGTGCTCGTTGGTTGGTTTTAAAACAGGGTTTCTCTGTGTGGCCCTGGCTGTGGCCCTGGCTGT
CCTAGAACTCACTCTGTAGACCAGGCTGGCCTCAAACTCAGAGATCAACCTGCCTCTGCCTCCCAAGTGCTGGGATTAA
AGGTGTGCGCCATCACAAGATCTTTCTCTCTCCTTCCTTTCTTTCTTTCTTTCTTTCTTTCTTTCTTTCTTTCTTTCTT
TCTTTCTTTCTTTCTTTCTTTCTTTCTTTCTTTCATTCTCTCTCTCTCTTTCTTTCTCTTTCTTTCTTTCAAGATTTAT
TTATTTATTACATATAAGTAACACTGTAGCTGTCTTCAGACACACCAGAAGTGGGTATCAGATCTCATTACCGATGGTT
GTGAGCCACCATGTGGTTTCTGGGATTTGAACTCAGGACCTTCAGAAGAGCAATCAGTGCTCTTAACCTCTGAGCCATC
TCGCCAGCCCCCACAAGATATTTCTTCTCTCTCTCTCTCTCTCTCTCTTTTTTTTTTTTTTGGTTTTTTGAGACAGGGT
TTCTCTGTGTAGCTCTGCTGTCCTGGAACTCACTTTGTAGACCAGGCTGGCCTCGAACTCAGAAATCCACCTGCCTCTG
CCTCCCAAGTGCTGGGATTAAAGGTGTGCGCCACCACGCCTGGCTTAAGATATTTCTAATGCTTAATTTGATTAGCAAA
GGCTCAACAAATGGGGAAATGAGACAGGAAGAGGAAAAGGCATTCAAACAATGTGTTAACAAGTCAGTAGCCACTGTCA
AGACTGAGGCTTAATTCCACTGGGGAAATTCTACGAGCCAAGGGGCACAAAAATAAAGGCTTTATACAGCTTCCTGTCA
GTTCTTTTTCATCGGTCATATATCCACTGGAGACACCTGCTTTGTTATGCAGACAGAACAGCAGACTCCTCATGCTCAG
AAAACACCTGCAAGCAAAGAACCACTTGCTAGTTGGTAACACCGTCCAGAGGTCTGTAAGAGCAAAGAAACACTTGCTA
GTTGGTAACACTGTCCAGAGGTCTGTAAGAGCAAAGAAACACTTGCTAGTTGGTAACACTGTCCAGAGGTCTGTAAGAG
CAAAGAACCACTTGCTAGTTGGTAACACCGTCCAGAGGTCTGTAAGAGCAAAGAAACACTTGCTAGTTGGTAACACTGT
CCAGAGGTCTGTAAGAGCAAAGAAACACTTGCTAGTTGGTAACACCGTCCAGAGGTCTGTAAGAGCAAAGAAACACTTG
CTAGTTGGTAACACTGTCTTGAGGTCTGTAAGAGCAAAGAAACACTTGCTAGTTGGTAACACTGTCCCAAGGTATGTAA
GAGCAAAGAAACACTTGGTAGTTGGTAACACTGTCCAGAGGTCTGTAAGAGCAAAGAAACACTTGCTAGTTGGTAACAC
TGTCCAGAGGTATGTAAGAGCAAAGAAACACTTGCTAGTTGGTAACACTGTCCAGAGGTCTGTAAGAGCAAAGAAACAC
TTGCTAGTTGGTAACACTGTCTTGAGGTCTGTAAGAGCAAAGAAACACTTGGTAGTTGGTAACACTGTCTTGAGGTCTG
TAAGAGCAAAGAAACACTTGCTAGTTGGTAACACCGTCCAGAGGTCTGTAAGAGCAAAGAAACACTTGCTAGTTGGTAA
CACTGTCTTGAGGTCTGTAAGAGCAAAGAAACACTTGCTAGTTGGTAACACTGTCCCAAGGTATGTGAGAGCAAAGAAA
CACTTGGTAGTTGGTAACACTGTCTTGAGGTCTGTAAGAGCAAAGAAACACTTGCTAGTTGGTAACACTGTCCCAAGGT
ATGTGAGAGCAAAGAAACACTTGCTAGTTGGTAACACTGTCCCAAGGTATGTGAGAGCAAAGAAACACTTGGTAGTTGG
TAACACTGTCTTGAGGTCTGTAAGAGCAAAGAAACACTTGCTAGTTGGTAACACTGTCCCAAGGTATGTGAGAGCAAAG
AAACACTTGCTAGTTGGTAACACTGTCCCAAGGTATGTAAGAGCAAAGAAACACTTGGTAGTTGGTAACACTGTCCAGA
GGTCTGTAAGAGCAAAGAAACACTTGCTAGTTGGTAACACTGTCCCAAGGTATGTGAGAGCAAAGAAACACTTGCTAGT
TGGTAACACTGTCCAGAGGTCTGTAAGAGCAAAGAAACACTTGCTAGTTGGTAACACTGTCCAGAGGTCTGTAAGAGCA
AAGAAACACTTGCTAGTTGGTAACACTGTCCAGAGGTCTGTAAGAGCAAAGAAACACTTGCTAGTTGGTAACACTGTCC
AGAGGTCTGTAAGAGCAAAGAACCACTTGCTAGTTGGTAACACCGTCCAGAGGTCTGTAAGAGCAAAGAAACACTTGCT
AGTTGGTAACACTGTCCAGAGGTCTGTAAGAGCAAAGAAACACTTGCTAGTTGGTAACACTGTCCCAAGGTATGTAAGA
GCAAAGAAACACTTGGTAGTTGGTAACACTGTCCAGAGGTCTGTAAGAGCAAAGAAACACTTGCTAGTTGGTAACACTG
TCCAGAGGTATGTAAGAGCAAAGAAACACTTGCTAGTTGGTAACACTGTCCAGAGGTCTGTAAGAGCAAAGAAACACTT
GCTAGTTGGTAACACTGTCTTGAGGTCTGTAAGAGCAAAGAAACACTTGCTAGTTGGTAACACTGTCTTGAGGTCTGTA
AGAGCAAAGAAACACTTGCTAGTTGGTAACACCGTCCAGAGGTCTGTAAGAGCAAAGAAACACTTGCTAGTTGGTAACA
CTGTCTTGAGGTCTGTAAGAGCAAAGAAACACTTGCTAGTTGGTAACACTGTCTTGAGGTCTGTAAGAGCAAAGAAACA
CTTGCTAGTTGGTAACACTGTCTTGAGGTCTGTAAGAGCAAAGAAACACTTGGTAGTTGGTAACACTGTCTTGAGGTCT
GTAAGAGCAAAGAAACACTTGCTAGTTGGTAACACCGTCCAGAGGTCTGTAAGAGCAAAGAAACACTTGCTAGTTGGTA
ACACTGTCTTGAGGTCTGTAAGAGCAAAGAAACACTTGCTAGTTGGTAACACTGTCCCAAGGTATGTAAGAGCAAAGAA
ACACTTGGTAGTTGGTAACACTGTCCAGAGGTCTGTAAGAGCAAAGAAACACTTGCTAGTTGGTAACACTGTCCAGAGG
TATGTAAGAGCAAAGAAACACTTGCTAGTTGGTAACACTGTCCAGAGGTCTGTAAGAGCAAAGAAACACTTGCTAGTTG
GTAACACTGTCTTGAGGTCTGTAAGAGCAAAGAAACACTTGGTAGTTGGTAACACTGTCCAGAGGTCTGTAAGAGCAAA
GAAACACTTGCTAGTTGGTAACACTGTCCAGAGGTCTGTAAGAGCAAAGAAACACTTGCTAGTTGGTAACACTGTCTTG
AGGTCTGTAAGAGCAAAGAAACACTTGGTAGTTGGTAACACTGTCCAGAGGTCTGTAAGAGCAAAGAAACACTTGCTAG
TTGGTAACACTGTCCCAAGGTATGTGAGAGCAAAGAAACACTTGCTAGTTGGTAACACTGTCCCAAGGTATGTGAGAGC
AAAGAAACACTTGCTAGTTGGTAACACTGTCCCAAGGTATGTAAGAGCAAAGAAACACTTGCTAGTTGGTAACACTGTC
CAGAGGTCTGTAAGAGCAAAGAAACACTTGGTAGTTGGTAACACTGTCCCAAGGTATGTAAGAGCAAAGAAACACTTGG
TAGTTGGTAACACTGTCTTGAGGTCTGTAAGAGCAAAGAAACACTTGCTAGTTGGTAACACTGTCCAGAGGTCTGTAAG
AGCAAAGAAACACTTGCTAGTTGGTAACACTGTCCCAAGGTATGTGAGAGCAAAGAAACACTTGCTAGTTGGTAACCCT
GTCCAGAGGTCTGTAAGAGCAAAGAAACACTTGGTAGTTGGTAACCCTGTCCAGAGGTCTGTAAGAGCAAAGAAACACT
TGCTAGTTGGTAACACTGTCCAGAGGTCTGTAAGAGCAAAGAAACACTTGGTAGTTGGTAACACTGTCCAGAGGTCTGT
AAGAGCAAAGAAACACTTGCTAGTTGGTAACACTGTCCAGAGGTCTGTAAGAGCAAAGAAACACTTGCTAGTTGGTAAC
ACTGTCTTGAGGTCTGTAAGAGCAAAGAAACACTTGGTAGTTGGTAACCCTGTCCAGAGGTCTGTAAGAGCAAAGAAAC
ACTTGCTAGTTGGTAACACTGTCTTGAGGTCTGTAAGAGCAAAGAAACACTTGCTAGTTGGTAACACTGTCCAGAGGTC
TGTAAGAGCAAAGAAACACTTGCTAGTTGGTAACCCTGTCCCAAGGTATGTGAGAGCAAAGAAACACTTGCTAGTTGGT
AACCCTGTCCCAAGGTATGTAAGAGCAAAGAAACACTTGGTAGTTGGTAACACTGTCTTGAGGTCTGTAAGAGCAAAGA
AACACTTGCTAGTTGGTAACCCTGTCCAGAGGTCTGTAAGAGCAAAGAAACACTTGCTAGTTGGTAACACTGTCCAGAG
GTCTGTAAGAGCAAAGAAACACTTGCTAGTTGGTAACACTGTCCCAAGGTATGTAAGAGCAAAGAAACACTTGGTAGTT
GGTAACACTGTCTTGAGGTCTGTAAGAGCAAAGAAACACTTGCTAGTTGGTAACCCTGTCCAGAGGTCTGTAAGAGCAA
AGAAACACTTGCTAGTTGGTAACACTGTCCAGAGGTCTGTAAGAGCAAAGAAACACTTGGTAGTTGGTAACACTGTCCA
GAGGTCTGTAAGAGCAAAGAAACACTTGCTAGTTGGTAACACTGTCCAGAGGTCTGTAAGAGCAAAGAAACACTTGCTA
GTTGGTAACACTGTCCCAAGGTATGTGAGAGCAAAGAAACACTTGCTAGTTGGTAACACTGTCCCAAGGTATGTGAGAG
CAAAGAAACACTTGCTAGTTGGTAACACTGTCTTGAGGTCTGTAAGAGCAAAGAAACACTTGGTAGTTGGTAACCCTGT
CCAGAGGTCTGTAAGAGCAAAGAAACACTTGCTAGTTGGTAACACTGTCCAGAGGTCTGTAAGAGCAAAGAAACACTTG
CTAGTTGGTAACACTGTCTTGAGGTCTGTAAGAGCAAAGAAACACTTGGTAGTTGGTAACACTGTCTTGAGGTCTGTAA
GAGCAAAGAAACACTTGGTAGTTGGTAACCCTGTCCAGAGGTCTGTAAGAGCAAAGAAACACTTGCTAGTTGGTAACAC
TGTCTTGAGGTCTGTAAGAGCAAAGAAACACTTGCTAGTTGGTAACACTGTCCAGAGGTATGTAAGAGCAAAGAAACAC
TTGCTAGTTGGTAACACTGTCCCAAGGTATGTAAGAGCAAAGAAACACTTGCTAGTTGGTAACACTGTCCAGAGGTCTG
TAAGAGGGATGTGGTGGCTTGAGTGAGAAATACCCCCAAAGTCTTGGGCATTTGAACAATTTGTCTTCAGGTGATGGGG
TGCAGGTGCTGGCTAGTTTTAGGTCAAGTTGAGACAAATCAGAGTTGTTAATTGGGAACAAAGAACCTCAATTGAGATA
ATGCCTTCACCAGATTGGCCTGAGGTCCATGTTCTTCTTGATTGATGATTGATATGGGAGGGCCCAGTTCAATGGGGGT
CCCTTGGGAAGGTGGTCCCGGGTGCTACAAGAGAACAGCCTGTGTGAATCATAAGGACTAAGCAGTAAGTGGTGCCTCT
CCGTGGCTCCTGCATCAGTTTCTGCCTCCAGGTTCCTGCCTTGAATTCTTGCCCTGACATCCATGAATGGGTTACAAGC
TGTAGAATTAAATAAATGTCTCTTCTTCAAGGTGCTTTTGGTCATGGTGTTTAACCACAAAAATAGAAATCCTAAAACA
TCGGTGCTGTTTTGGGGAGGTTTAGGTAATGCTCTGTTGATGGTCAGATCAACGGAGTCTTTTCCTGAGGCTCTGATGT
ACGGAGAATGAACCTGCTTGGCAGACCCAGTAATGGCCCTGGGCCAGGGCTTTTGGGTGGGGATCTGTAGCTTCTGTTC
TCTTCCCAGCGTGTGGGGCTATGGTTATCAGTCCAAGGCCACTGTGTACCCGGTCTGTAATGTTCTTGAGATACCCCTT
GCTCCCCTTGTGCAACACTGTTTGATTACCTTCCTGTTGACTTCGTCCCTTCGTAGGACAGTTTCTGCTGACTTAGTCC
CATTTCTTCTCTCAAAATGGTGTCTGAGATGCTGTAGTTGCTTGTCCTAAGACATAGCTCGCATGAGACCTCCCGACCC
CAGATTTTCTCTCTATCTTCTTATCTAATGTCCATCTCCCGCTTAGGACCATGTTACTGAAGAATGACTCTCCGACCCA
AGTCAGTGCAGTCGCGCTTGGACAAAGCAGATCACTGGGGTTGGAGTTTAAGACTCAAACGAAACGACTTGCCTACTTG
CAGTTTGCTCTGCCTTGTGTTTGTAGTTCAAGAGCTGAGGAAGCTGCCAGCTGCTATGCTGCCCCGACCACTGTGGACT
CTGACCCTTTGGAGCCATTGTAAGTCTCCTTCAAGTAACAAGACAGTCATCCATGAAAGAAGCATGGACCAGTTGTGAT
GACTCATTGCTACAATCCAAGCATTTGCTAGGCTGAGATAGGAGGAACAGCACAGGCACTAGGCCAGCCTGGGCTACAT
AATGAGTTCCAACCCAGCCTGGCCATAGAGTGAAACAGAATCACAGAAAACCAAAGAGGGAGAGAGAAAGAGGGAGGAA
AGAGGGAGAGGGAGGGAGAGAGAGAGAGAGAGAGAGAGAGAGAGAGAGAGAGAACGAACGATGTTGGTGGTAAGGATAA
AGGTAGAATATTAACTCCAGAATCCTGAAACAACAGACATTGAAGCCATTGTTTCTGGAATGGGAATATATCTCACTTG
ATGGCGCCTACTCAGCCCAGAAGAGTCCTATACTCCTCCATCTTTCCTCCATATATAGGCTCTCATGGCCATGACTCAA
AAGCCTCTTAGGTTTCTCTTCTTTTTAGGACCTAGATACAATTGGCTTTTCTAACAATGTAGCCCCCTACATTTTTTGG
CAAGGAAGCCACTTTTTTCTCAGAGTGTCTCCTTACACGATCTACTTAAACACAGCCGGGAACAATCTACATGTGATGT
TGGCATTCTACTTTAGAGCCCTCTCCCTGGAACTCACACTGTCGGTAGGCTGTACTTTCCAGCTAGCTGAAGCTATCAG
CTTTCATAGCTGCTAGGCCACTTCAAGTTAGCTCTGTAGCAATAGAGGCTGTTTGCTAAGCTCTCTTGTATTTATACAT
ATTCACTTGAAATGCTGGGAATTTAAATATAAAACCCTTTAAGTAAAACAAACGAGCCCTTTTCAACTAAAAAAAGAAA
AAAAAAAACATCAAGGTGGGTGATACCTGAGGAATAACAGCTGGGGCTGGCACCTGACTGTCATATACTTGAGCACACA
TGTGTACCTGCACACATGTGAACATACATAACACACGCCTCAATAAAATGTATGTTGTGGTGGGGCTGCAGAGGTGCGT
CAGTGGTGAAGAGAATTTGCTACTCTTCCAGAGGACCCAAGTTTTGTTTCCATGTTAGACAGCTCCCAACTGCCTGTAA
CTCCAGCTCTGGAGATTTGATGCCACCTCCTGGCTTCTATGGGCATTACATTCATGTGCACAAACACACATAATCAAAG
TAAAAATATACTGTGTTAAATGCATAAACATATACCATGGTCATTTATCAGATCGCCTATAGACTGCTCTGAAGGGCAT
TTTCTTGATTAGTGATTGATGTGGGAGGGCCCAGACCATTGTGGGTGGTGCCACCCCAGGGGAGGTGATCCTAGGTGAT
CTAAGAAAGCAGGCTTGAGCAAGCAGTGGGTGTAAGCCTGGTTGTCTTTGGCCTCTGCTTTAGTTCTTGCCCTGACTCT
CCTCAGTCCTTGGTGACCTATCAAGTATCCTACAGATATGATGACAACTTCAGGACAGAACTGCATTTTAGACATCAAC
TGTGGCTTCTTTTTCACAGCTCTAATAATTTCTTCAAGGCACTGCAGGGTCTCATGTATCCCAAGATACCTCCTGCTTA
GCTAGGATGCAACCAGTGTTATATGGCCCTGAAGATGGAACCCAGGGACTTTTATGCTAGGCAAGTGCTCTACCAACTG
AGCTACATCCTCAGACCCTTACCTCCAATACTAAGTATTTCCCTTCTTTTCCCTAGTGGGCATTTACCTCTGCGCCAGT
GTTATCGATATCCTGTAAATGAAAAAAGTTAATGATACCTTTAAAGCAGGACCATTATAATCACTACAGTGACCAGCAA
CCCTAAATATATTGTACTTAAAAGAATAGAGTTTGTGTTGGGCGTGGTGGCATACCTTTAATCTCAGCATTCCGGGAGG
CAAAGGCAAGATCTCTTGAGTTCAAGGCCAACCTGTCCTACACAGTGAGTTCCAGGACAGCCAAGGCTATGCAGAAACT
CTGTCTCAAAAAATAAAATAAAATAAAATAAAGCTGGGGGTGGCTAATAATTCAGCATTTTATCAATAATTTACCCTTA
GCAACTTTTTTTTGTTTTTGTTTGTTTGTTTGTTTGTTTGTTTTTCGAGACAGGGTTTCTCTGTGTAGCCCAGACTGTC
CTGGAACTCACTTTGTAGACCAGGCTGGTCTTGAACTCAGAAATCCCGCCTGCCTCTGTCTCTGCCTCCTGAGTGCTGG
GATTAAAGGCGTGATCCACCACTGCCCCCGGCTACCCTTAGCAACTTTTAAGAGGTAACATAAGAATGGGGTTTTGGTT
AGAACCTAAGAGTTTTCTTACTGTAGATAGGCCAGAATAGTCAGATATTACCTGGGGACAGTGAAAAATGAGAAAGGGC
ATGAGCTATCAAAGGTGGCAGGATCTAACTGGATTTTTACAAGGAGAACGACCAGTAGTCAGACTACAGGTTTGGCTAA
GTAAAGATTCTTTGTCACAAAATGCCGGAAGTACACATCAGAAAAGAAAAAAACCAACTTCTGTAATAAACTGTGCCGG
GAAAATTGGATAGCCATACATAAAAGAAGGAAACTAGATCTTAATCTCTCTTGCGTTACACAAATATCAACTCCAAATG
GATTAAAGACCTTAATGTCAGAACTGAAATCCTGAAACCAAAATAGTCAAGGGCTTTCTGAATAGGGCCCTAGTTGCTC
AGGATATAATACGAACAACGGACAAATGAAATGGGATTGCGTGAAGTTAAAAGGTTGTGCACAGCAAAGAAAAATTAAT
CAAATGAATAAGCATACAGATGGGAGAAAATCTTTGCCAATTATACACCTGATAGAGGATATCTAGAAATACTTTTTTT
TGGGGGGGGGGGGGTGGAGTTTCAAGACAGGTTTCTCTATATAGTCCTGGCTGTCCTGGAACTCACTTTGTAGACCAGG
CTGGCAACTCAGAAATCCGCCTGCCTCTGCCTCCCAAGTGCTGGGATTAAAGGTGTGCTCTACCACTGCTCGGCTTAGA
AATACATATTTTAAAAGTCTTGAATGTGAATATAACCTAATCAATAATCTAGCGTGCCTTTCATCACCCAGTCTTCTTT
CCGTTACTGTTTTTGGCTTTGGTAAGGATTGATTGTTCATCCACTTTGGGGAGTCAGACCTTGACTTTCTTAACATCCT
CCCACCCACAAGCCGCTATTAGCTTCATGCGCTGCCCCGGCACCGTCAGGAGAACTCGCCACTGGACCCCCTCGCCCCA
CTTCTGCACGTTCTGGACTTAAGGCTGATCGGTCCGGCCGCCCGCAGGCCCTCGCCACGAACGATGAGCTGCGCCGCAG
CCGAAACTGTTGCCGCAAACCCAGACCCCAGAGCATCTCGGGAAAGCGTGCCCAAGGACCGCCCTCTTCCGACCGCTCC
GCTTCCCAGAGCGGGGGCCCCGATGACGCCACTTCCTCCGGAAGAGGTGTGGCGTCCGCTCGGTTGGCGCGAAAGTCGG
TTCCCGGTCTTGGCCCTTCCGGGTTCTGCAGCGTTAGCTTCGGGTTGGGGGCGCCGCCGAGGGCGGGGGCTGCGCGCCG
GGCTGGCGCCCGACCCCAGCCGCCGTCCTGCCGGCCGCGCGCCCCCCGACTTGGTGCCCGTGGGCCCTCGGGCTCCACC
TAGGTGAGCGAGCTTCGGCTCCTCCCGGCCGGCGTGGCCGCCCGGCGCCTCTTGCGGCCTTCGCGCCTCTCCTAACCGG
CGGGTGGGCCTCAGGTCCCTGGGGAGCCGCGGCCTGGACGCTCCTTTCCCTGGGAGCTGGGCCGAGCGTGCACGACCTT
GCACGGTGGCTGCTTGCGTTCAGAGTTCGTTAACTTAGGAGCAAATCTTGCGGGCTGTGGGTACCCGTAGCCGCAGCGG
CGCCCTGCGTCGTCTTAAGCCCCATGCAAGTGAACAGTGAGTGACTGAGGACCAAACCAAGGGTCTCAGGGGATTCAAT
CAGGAACGTTCATTTTGTTCTCACGTTTGCAAGGATCCGTGTCCTTTTTTTTCTTCTTCGACTTTGCCCACCTTCTGGG
GCCCTTTGTAAACATCTTGAATGAATTGATGAACCAAGCCAGATAGCTATGTCTTAGGCACTCTTGTCTGCATAAGAAA
ATAGTTGATTTGCAGGCCGGGCGGTAGTGGCACACTCCCTTTTTTGCCAGCTTGAGTGAGGCAGAGGCCGAGCATCTCT
TGAGTGCCAGGCAGACCTAGTCTACAGAGCGAATTCCAGGACTGCCAGGGCTTGTCTCAAAACTAAAAAAAAAAAAAAA
AAAAACCAAAAACCCTACAAAAATAGTTGACTTGTACATTTAATTCCCTGATTGACTGACTGACTTTTGTTCTTCTTGA
CTGCCTTATTCTCACAGTTCAGCTATGTCCAGAAATGACCAAGAGCCGTTTCTTGTGAAGTTTTTAAAGTCTTCGGACA
ATTCAGAATGCTTTTTTAAAGCTCTTGAGGTAAGAATTAATGGATATGTCTTATTTGATACTTGAGCTCATTTTCATGT
TTTAAATGTTTTTGCTTTGTTGGATTTTATGTCTTAGAAGTAGATAATTTTAATGAAATTTTGCTATAGCATATGCTTA
ATACAGATAAATCACTGCTATATAATTTTTAGTTGATGCAAGTTTACCATTATATTTTTTGGGAGGATTGAAATCTATT
GTGACAATTGGATGTTAACCTACAGTTTGATCCCCTACCCCCGCCCGGTCTGTGTCTGTTTGTGTGTGTGTCTGAGCCA
TCTTCCCAGTCCTCTGACTTAGAGTTCCTGACTTGTTTTGTTTACTAACCCCATTGATCCTTTAAGCACTGAAAAACGG
AGTCACAATGCAATAGTTTTGGTTCATTGCTTGCTCTCTCTCTTTCTCTCTGTCTCTCTCTCTCTGTCTCTCATACACA
CACACACACACACACACACACACACACACACACACACACATATTCATGAATGAATGAATGATGGGAGTAGTTTTGAGCT
TTGCCATTCACAAACCCTAGTAATGTCCTTGTAGCTTGGGAATCTATTAGAAATGCAGAGCCTTGGGATCCCAACCCAC
ATTTGACAATTAGAATATGCATTTTAAACAAAAGTCCCAGTGCATATTCAGGTTTGAGAAGTTAGCTGTTTTTTTTTTG
TTTGTTTGTTTGTTTTGTTTTTTAAAGTTCACTCACTTAGCAATCATCAATTGAGTGTCCGTCATCTGACAGGTTTTCC
TCTAGCATAATACTTGGGGTACACCAATGATCAGAACAAGCAAATATCTCTCCTCTTTCAGCATACATAGAACAGGAAA
TCGGTATTAATCAGGTGATAAGCAAGAAACTACACAGTATGGGGGCAGTGAAAGAGTTGAGACTGAGTCTCACAGTGTA
GCCCAGACTGATCTGGAACTCTGTGCTTCTGCCTCAGTTTCCTGCATGCTGAGATTACAGGTGTGTGCCCCAAAACCTA
GTACCTATGTAATGTGTTATAAGGGGTCTAGTACTGTGGGAAAATGAAAAAGAAAAAGCCAAAACAATTTAAGGTTTTC
CAGAGGATGTTTGTTGGAGGAGAAAGACATGGAAGGGTTACAGCTTAAAACTGAATTTAAAAAAAAGCAAACTATTTCT
CTTAAGATTATAGACTAGGGTCTTGTATAAGGTACTAGGTATCCGGATATGCTTATAAACAAAATACATCATGGAATGT
TCATTTGTGTTGGTGTGTGGGGATGGTGTTTATGTGGGTGCTCTTGTGCACATGCATTCGGAATAGTATCTGCATACCA
CAGTACATGTGTAGATGTCAGAGGACAACTTGAAGCAGTTGGTTCTCCTTTTACCCTGAGGTTCTCAGGCTGAATGGCA
GGCACTTTTACCTGATGAGCCATTTAACAGAACCAACACATTCCTTCTCACAGGGAAGAAATAAAACTATGTACATTTA
CAAAAAGAACCATTTTTTTTTCCTTCATAAAATGTAGTCAAGTGGTAGAGGGAGTGTAGTTAGAAAGCAGGATGACCAT
GGGAATATTGCTTAGTGGTAGTTGTATAGTGTGCATAAAAACCTGGTTTTAGTTCCCGGTGCTGCAAAGCAAACCCACC
GTGGTATCTAGAAAGATCTAGACAGGTATCTCTAGGAAGTGACAGTGAAGCAGGCGTTATTGCAGTGAAGAACCACTTC
CAATGAAAGCTTAAGAAAAGAGTAGCTCCTGTAGTAGGACCAGAAGCAGAACCGCTTAGTAGGTCATTAGTATCTTCCA
GGTGTACGGTTTGGGGATGACTGGAATTAGCCAGGAAGGTGGATGTAAAGATGAGACTTGAGATAAGTGAGGGCTAAAT
TTTATGATTTTGTAGTCATGAAGTTGTATGGTGCTGATGCATCTTTAATCCCAGCTCTTGGGAGGCAGAGAGATCTCTA
TGAAATGGAGGCCAGCCTGGACTACGGAGTTAAGTTCCAGGACAACCAAGGCTACACAAAGAAACCCTGAATGGGGGGC
GGGGGGGGGGGGGGAAGAATTGGGACTGATCCATGTGCCCTTTCTTTTTGTCCAGTGGTTCATTTCTGCTATATTCCTC
AGTTTCTCTCCATCTTATTTTTTTGAGATAAATTTCTCACTGTTGTAGAGCCCACTGATTTGCTAGACTGGTTGGCCAG
GGATCTGGTGGGCTCCAGTCTCTGGCACCCACCCCACTGCCCAGTATTGGGATTGTGAGTCCATGCCTCCACACTAGGC
TTTTTACATGGGGCCAAACTCAGATGCCCAAGCTTACACTGCAGGCATTTTACCCACTGAACCCTCTGCTCAGCTCCTG
GGTGGTTTCTTTAAAAGGATTACTAACTGTGAAGAATAGAGAGGAGACCAGTTCAGAGGCTTCTGCATGGGTCTAGGTG
AGAAGCATGCATGGGTCTAGGTGAGAAGCTTTATTTAATAAGGAAAGGGCTGGGTTTGAAATTAGCTCTGAGCACTTAG
TAGGACTTAAATTTAGGGTACTAATTTCTACTACAATCTTACTTGTTTTTTCTTTCAGTCAATAAAGGAATTACAATCA
GAAGATTATCTTCAGATTATTACAGATGAGGAAGCATTGAAGATAAGAGAGAATGACAAATCGCTTTATATCTGTGACC
GTTTCAGTGGCACTGTTTTTGATCACCTTAAACAGGTTTGCTGTTTCTAGAATACTTATTTGCTCTTTCACCCTCTCTT
TTAATCTGTGTGTACCTGTGCTTGTGTGTGATGTGTCTGTCTGTCTGTGTGTGTGTACATGCATGCGTGTGTAGGGTCT
CATTCTTCATGAAAACTATCATGAGTGCTACGAGTACCTTGGGATTCTTTGGGCTTGGGGTTCTTAGATATCTGAGCTG
CTTTATTGGCTGTTGCTTTTTTGAAAAGCATACTTGAATGTTTCTCAGTTGTTATAGCATAAGATTATTGCATTAAGAT
TGTATTTCTTGGGAAAGGTGCCCTAGTGGTTCAGGGCACGTACTTCAAGGGATCTGAATTTGATTCACAGCACCCATGT
CAGGTGGCTCATGACTGCTTATAATGCCGGTTCCAGTACCTCCAACTCCAGGTTTATCTGTACAAAATGTGTGTATACC
TACATAAGGATACATACATACATACATACATACATCATATAATTTAAAATAATAAAAAGTTTTATGTTGTAAGAGTTTT
TACCTGCATGCATGTTGGTGCACCATGTGCACACTTGATACGCTTGGGGGTCAGAAGAGGGCATTGGATCCCTTAGAAT
TGAAGTTACAGGTTGTAAGCTACTGACTAAGGTACTGGGAACCAAACCTAGGTCCTTTGTAAGAACAATAAGTGCTCTT
AACTTTTTAAAATTAATAATGATAATAATAAGACAAGGAGGAAAGTTTGTTTTAAGTGGTTGAAGTTTTGGAGGCAGTT
ATGAGCGCTCTTGTGGTTTTGCTTTGGAGGGTGCGTGTCTATTTAGTGCTTGCTTGAGAAATTATGTGATAGTCTTTTT
CTCCTAACATTTCATGGTTTTCTGCAGTAGCTGTTAGCTTCTGTGAGTAACTAATAAAATGTCCCAAGTTTAATTTTGT
GATTTTGTCCCAAACTTTTTACAGCTTGGCTGCAGGATTGTTGGTCCTCAAGTGGTCACATTTTGTATGCGCCACCAGC
AATGTGTCCCAAGAGCTGAGCATCCAGTCTACAACATGATAATGTCTGACGTAACTGTATCCTGTACAAGCCTGGATAA
AGACAAACGGGTAAGCACTGAAAGCAGCCGGGCTGCCCCCACCTGACTTACCCAGGGCTCAACAGCAGCAGGCCTCACA
CGTGGGGTTCACAGCTACAGGCCTCACACGTGGGGTTCACAGCCAGCAGACTTGTGCATGGCTCCCAGTGGTATTTTTT
TCTAAAGATTATTATATAATTCCATCATTTCCTCATCCCTTTTCCTCCCTCCAAATCCTCTCATACACCATTCCTTGCT
TTTTTTTTCAAGTTTTCAAATTTATAATCTTTTTTTCATTGTTATATGCATAAATATATACATAATCTTAGATAAATAC
AACCCGCTTAGTCTGCATAATGTTACTTATATGTATTGGTGTGCTCTTCCCTGGGGAAAGTGGAATATGATTGTAGAGA
CACTGGTTAAGTGCTTTGTTTTGATTTAAAAGAAAAGTTACTTTTTTTTCTACAGTCAGTGAGTTTTGTTTTAAATCAT
TTAATTTTTGCAGAACAGAAAATCTTTCAAGAAAGGATTCTAATCTGGGTGTTGTAGTGCATGCCTTTAATCCCCAACT
CTTGGAAGGTAGAGAAAGGCAGATTAGTGAATTCCAGATCAAATAGGGCTACATATTAATAAGACCTGTCTCAAAAAAA
AAAAAGGAAAGGAAAGGAAAGAAAAAAGAAAAGTGGCTGGCACCTTTAATCCCAGCACTGGAGAGACAGAGGCAAGTGT
GTCTAAGTTCAAGGTCAGCATGGTCTAAGGCCAGGATAGCTAAGGCTACACAGAGATGCCCTGTCTCGGGGAAAAAAGA
AATAATCAGAAGCAACATAGTTTTGAATTATGGAATTTAAAAGAATAAATAAGGTCACTTATTAATGATAATAATTCTA
CCTGAAAGTGTTTCTGTAGGTGACTGAGGGTGCTTAAACTTAAGAAGGCTATGATGTCATTAGCAATATTAATCTGCAA
GACCACATTGTTATAAATGTGGTTGATTAGAAAGTATTGTTAAAAAGTATGTGACTGTTCAAGAAAACAGTTCTTTTCA
GGTTTTTTGTTTTTTTGTTTTTAGGGGAAGGCTGTGGATTGAGCCCAGAGCCTTGTGCATGGTAGGCAAGCATTCTACT
ACTGAGCTACATCACCAGCCTACAAATAGCGATTTTTAAATTTAAAATTGATACACATCTTTCCAAAAATAATTACTTA
TTTTTTTTAGTAGCAAAAAACATTTGTATGTTTAGTCTATCAACAGTAAATGATACTTATGGGGGCTGGAGAGATGGCT
CAGCGGGTAAGAGCACTTATTGCTCTTAGAGAGGACCTGGGTTTGGTTCCCAGTACCTGCTAGATAGCTCACAACCATT
TAAACCTTCAGTTTCAGGGGGTCTATCTGATGCCCTCTTGTGATCTCCAAGGCATGCAAATAATGTATAGACATATACT
CAGGCGAAAGAAACACTCATACACATAAATTTTTTTAAAAAAATTACGTGATTTTTTTTTTTTTAAAATAGAAGAATTG
TTGTTTTCTCCAGCATTAACATATTTTCTTTTGGGTCTATAGGAAGAAGTTCATAAATACGTACAAATGATGGGTGGAC
GAGTATACAGAGACCTTAATGTATCAGTCACTCACCTTATTGCAGGAGAAGTTGGGAGCAAAAAGTACTTAGTTGCTGC
TAATCTGAAGAAGCCTATTTTGCTACCTTCTTGGATAAAAACCCTTTGGGAGAAGTCACAAGAAAAGTGAGGCACATTC
ATATGCTTTGTCTGTTTGAAAAAATGATGCATTTGGATAGTTTTGACGGTGTTTGGTTTTCATGGGGAAGCTTTATGGT
ATCTCTGGACTTGTTACTGAGAAACAAAACAGGGTCTTTGTTCATTTCTCCAGTGACAGTAGGGGAGATGGTCCATAGA
TTCAGCAGTGCCTAGAGGGCAAATGCTTTGATAATGAAGTTCTTTTCTGCATCAAATACATCTAAAATAATTCAGTTTG
GCTTTTGTGTTGCACTGTGTAATACTGTTCCCCAGTATTAAGATGGAGAATAAAGTACCTACTTGGGCAGCTCTGTTCC
CTGCTGGGTAGGGTGGAGGTGCAGAGAGGGGTAGAGGTAAGATTAATATACATATATGTAAGCCATCTGCTGAGAGGTG
ATCATTCCGTGTACATCGAGCCTATTTTCTGCACTGGGCTGGACATTAGATGTATGTAGAGATCAGAACAGGATAACAG
TTTACCTTATGGAGTTTGGAGGTACACTTCTGTTTTCCCTAAACTTGGGTTTATAGTAATAAATCCGGGTATCTGAATT
ATATCTGCATTCTGTGGCATTTTTCTGCACTTATTTCAACATTATTCAGCATGTACTCAGTACATTTCCTAGTTAGTAC
CAGCCTGGTGAAGTTTAGCACACCTACTTATTGTGGCAACAAAATGGCAGTACTATTGTTGGAACTCTGCCGAAAAGCA
TTCCTTTTACTTATCAAGAGTAATGGAAGAATGTGTCTGTTTTATACCAGACAACCTGTGTAAACGTTAGGTTTTTAAG
TACAGATCCAGTCTTATAATTCTGCCAAATGCTCCCCATTTATTAGCAAGGACTTAGGAGTTAATAGTGCTTTCTTTAA
GATAGTCTTGGGACTGTTGCTGAAATACAAGTTTTTATTAGAGACTTGGGACTGAGCTAGATGAGCAGTTAGTGTAATC
CTCAATGTCAGTTTTCCTTTCTGAAAAATGTTTATAGATCCTAAAGAATAGCAACTGAGCAGACAGGGAGAGCAGTAGG
AGCTAGCACGGTCTGAGTACTCGGGCTCAGTTGTCTCCTCTTCTTACTTTCTCCAGAAAAATAACCAAGTACACTGATG
TAAACATGGAAGACTTTAAGTGTCCTATTTTTCTTGGTTGCATTATCTGTGTGACTGGCTTAAATGGAATACACAGAAA
GACAGTCCAGCAACTCACGGCTAAGCACGGAGGCCAGTACATGGGACAGTTGAAGATGAATGAGTGTACACACCTCATT
GTGCAAGAACCAAAAGGTAAACAATGCCTTAGATGTGGTTGAGCGTGCAAGTCCTGTTTAAGCACTAATTCTCTGTAGA
CCACCTTGTGTTCTGATGTGTCATTGCCTTAGTTTATCTGGGTTTTTTTTTGCCTTTTAAATTCTTGAGAAGATAGTTT
GGGTCACAGTCTAGAAAAATTAAGGAACTGTCCTTGTTATTGTCCAGAATATGGAATAAACATAAAATATCAAAAGTTG
CCCATGATGTTTCACTACCTGACTTAGACCTTGATGAAAAAAAAACAAAACTCTTCACAGCATAACAAATATGTATAGC
TGAGTGGTATTCTTTCTCATGACTAACTTCAGAAGCTTAGTTAAGCACTTAGTGGGATGAATTGGTTTATATTACTGCA
TTTGGATTGGGGTGCGGGTGGGTGGTATGGGTAGAGTTCCCGAGTGCTGGGCAAGTGCTCTACCACATTTTCCAACTAG
TCTATGAACTAGAATTTCTTTCTTTCTTTCTTTCTTTCTTTCTTTCTTTCTTTCTCTTTTTCTTCTTTTTCTGAGACAG
GGTTTCTCTGTGTAGCCCTGGCTGTCCTGTAACTCACTCTGTAAAGTTTTACTATTGAGAAACTTTAAAATTATTACTG
TTTTTGATATTTCGGGAATTTTATTCTAGGGAATAGTTTTGCTAATACTCCTAAAATCAGGGTTAGGGTTTATACAAAT
ATATAAGAAGAGTCTTGCTGTATTTTTAGTAACTCCTTTGTGTTTGTACTGTTTTACTAAGCTACCTTGATAAGAAGCA
TTTGAGAGTGATTGTTAGTTACTTGATAATTGTCACTTGATCAGGAGATTGGTGTAATGGTTATGATAGGACAGTTACT
GTGGCTTTTCTCAATCTAAAGGCAGTCTGTTTATCTATTAACTATGTTTTAAGGTATGCGGGATTGAGCCTAGGTCTTG
CTTATGCTAGGCAAGTGCTTTCCCACTGAGCAATGACCCTAGTCCTGCTTTCTGTTGGAGACAGGCTCTCGTCAAGTTT
GCCATGCTGCTGTGAGATGTATGATTTTCCTGTCTTACAGAATATAGACTCTACCAGGCCCTGCTCCCTTGACTTTATG
GAGTACCAAAAGTTCTCCATAGAGCGGGGTGTCAAAGGAGGAGGAGGAGGCAGGAAATCAGGGCTACACATCTTTGTCT
AGTCAAAGATAGTATTTATTACGTTGCATACATTTATGGTATCTGCAGTTGTATTATATGTTCTTATTAAACAACATTA
ATAGCCGGGCAGTGGTGGCGCACGCCTTTAATCCCAGCACTTGGGAGGCAGAGGCAGGCGGATTTCTGAGTTCGAGGTC
AGCCTGGTCTACAAAGTGAGTTCCAGGACAGCCAGGGCTATACAGAGAAACCCTTTCTCAAAAAACCAAAAAACAGAAA
CAAACCAAAAAAACAACATTAATAATGTCAAGCATGATGGTGTCTTCAGTCCCAGTGCTTGGGAGGCATAGGCAGGCAG
ATCTGTGTGAGTATAAGTCCAACCTAGTCTATCCAGGCTAACTAAGCCTACAAATGAGACCCTATTCCAAAGCTGGTTG
GCTGGAAACTTGACTCAGTGGTTAAGAGCACTGGCTACTCTTTCAGAGGACCCTAGCTTGATTCCCAGCACTTATATGG
CAGCTCACAACTCTCTGTAGTTCCAGTTCCAGGGTGTCCAACACCCTCTTCTAACCTCCTTAAGCATCAGACATGCAAG
TAGTACACAGACATACATGCAGACAAAACACCCAGACACATTAAATAAGAAATAAAAAATTCGCTGGGCGTGGTGGCAC
ACGCCTTTAATCCCAGCACTCGGGAGGCAGAGGCAGGTGGATTTCTGAGTTCGAGGCCAGCCTGGTCTACAGAGTGAGT
TCCAGGACAGCCAGGGCTACACAGAGAAACCCTGTCTCGAAAAAAAAAAAAAAAAAAGAAAGAAAGAAAAAATTCTTAA
AGGTAAATATATTTACTGGTAAGCTTTAATCATTACTTTTGTTTGGAGTGACTTTTAAAAAAATAATTTACTATGAACT
AACTATATAGTCTATGTATTTCTGTATTTCTTTCGTAATGAACACAGGACAAAAATACGAATGTGCCAGGAGATGGAAT
GTGCACTGTGTGACCCTGCAATGGTTTCATGACAGTATTGAGAAAGGCTTTTGTCAGGATGAATCTATATACAAGGCTG
AAACCAGAGTAGAAGCAAAGATGGTGCCTGATACTTCAACACCCACTGCTCAGAGCAATGCTGAAAGTAAGTCTCTTTG
AGATTAAAATAAACAATTACTTGTTCACATGATAATTGATTGACTGGGGATGTAGCTTAGTGGGAAAGCTTTTGTCTTG
CAAGCCCAGGAACTCAGTCTTAAACACCAAGGAAAAAGTGAAGGAGTTGATTTGTTAACAGGACCATAGGAATATAGTA
GGGACTTTATTTTCTCCCTTTGCCCCTGGGTAGAATTAAATAAAATAGCCATGCGTAGACAGCTTTCCAGAACTTCTTT
TGTATGCCTTTTGGAATGAGAGATTGCTTTCTTTCACATCCTAATTCACACAAGAGGGGATTAAGAACACTAGTGTATG
GGCTTAAGGGAAGAGTTGTTCGCCCCTCTCCACATTTGTTTGCTTGGTTAATTGGTTTTTCAAGACAGGGTTTCTTTGT
ATAGCTCTAGCTGTCCTGAAACTCCCTCTGTAGATTGCTGAGCCCGGCATGGCCCTGGTGGGGACAGTTCTTGCCCCTG
GGACAAGACCCGGTGTGGGCCTCCTCAAGTGTTGATGGCTGGTGGGTAGAAGACTTGTTGTGTAGTAATGTACGTATTT
TCTGGACCAGCAGTGGGCGAGTCTTTCCTTCTGAAGAATGTCCTCCCTGTTAACTTTAATGATTCCATGATAATTAGAA
ATAGAGTCCAGGAGAGTGTGGCTGTGTCTCGGACCTTCAGGACAGCCCTTAAGGATATGGGAAAGAACTCTAAATACGT
GAGTTCAAAAATGTAAGGATGCAATATGAATTATGAGGGGCTTCACGAATCTAAAGGAACAGAGATAGCTACACTATGA
GCCTGCTTGTCAGAAAGATACAAAGGCAGGCGGATACAAATGTCCCAGGCAGATTCCTGGGACACAGCAAGATTAGGTC
CAAGTGTGGTTGAAATGATAATTTCCAGTTCTACCTAGCTAGTTTAGCATCTGTGTGTAAGAAACTAGCAGATCTCTGA
ATTCTTTTGCAATGTTAAAAGAAAATGTGTTTGTTGTCTCCTAAGAAACAAAAAAGTCACTGATGCTGATTCATAGGAT
AATTAAAAAAAGGAACCTGGAATAAATAACTGAATTGTTATGTAAATAAAAAACTGGGCTTTTGGTCTACCTGATGTGA
ACTAGCAGAAAGCAGTAGCAGATCCTTTTTAGAATTTTCTCTAGTGAGAGCTCTGGAGAACAAAAACATCTCTGCAAGA
ATTTTTCTTAATTTTCCAACTTGGTAGGAAGATAAAATAATTTTTTTATAGCAGTTTTTCTGACTATGGTCAGAAAACC
CATGAGCTATCCAGATAGCTTTTAGAATTTTTCCTAGCAGAGAGAACTGTCTCAACCAGAGTTTTTAGAATAGTGAGAA
AAGGCTGTCCAGAGCAGAGCAGAGCACATACACACTCATACACACACACACACACACACACACACACACACACACACAC
ACACACACACACACTAACTCTCACACACTCACACACTCAGACTGAGAGATCGAGCAGTCTGGAAAGCCATCTCAGCAAA
ACCTAGGCTGCCAGCTTGGACCCACAATTAGACTTCGAGTTGTTCTTTTCGCTGCTCTCAGACACTACTTCTCTCAGAA
CCCCTCTCTGCCAAGGCTAGTCCCTGGCAGTAGATCATTGAGCTGGCCTCAGATTGAGATTGCGTCCACTTTCTGAGCA
CTGGGATTAAAGGCGTGTCCACCATGCCCAGCCTATATTTTCTGTATTGGTTTACTGTAATGATTATAAATCATTACAT
AAAAATTCGGCCTGCATGAAGTCCTGAGTTTGATTACCTTTTGATTACTACTACTGCATTATAAAGCTGGATGTGGTGG
CACATACCTATAATACCAGCACTTGAGAGTTGGAGATGGAGGGATCAGAAGTTTAAAGTCTTCTTTGGCTACAAAGAGT
TCAAAGCCAACCTGGATTTGGTGAGACTCTGCCTCAGAAAACAAAACAAAAGGCCAGGGACAGTGGTATATAACCTCAG
TGCCAGCACTGCGGGCTGAGGCAAGTTCGATGTGGTCCTGATCTGGGTAGTGCAGTCCTTTTCAGCCTTCCTAATGCTG
TGACCCTTTATTACAGTTCCTCGAATTATGGTGACCCCGACCATAAAATAATTTTGTTGCTACTCTATAACTGTAATTT
TGCTACTGTTATGAATTGTAATATAAATATCTGTTTTCTGGAGGTCTTAGGTGACATCTGTGAAAGAGTTGTTTGACCC
CCAAAGGGCCTCAACCCACAGATTGAGAACCACATGCCCCTACATCCACATGCCACTCTGATGTGTCAGCTAGGCTAGC
ATATTCAGTCAATACTGAGGTCTTATTTCGGAAGTTCCATTTAGCAAAGTCTTCCTCCTTGGACCTTAAAGCCAGCCTT
ATGGGCTATAAGCCACTTTAAGGAAATATTAAATATAATTTCACCTAAAGAATGAAACTTTATAGCCGGGCATGGTGGC
GCGCGCCTTTAATCCCAGCACTCGGGAGGCAGAGGCAGACAGATTTCTGAGTTCGAGGCCAGCCTGGTCTACAGAGTGA
GTTCCAGGACAGCTAGGGCTACAGAGAAAACCTGTCTCAAAAAACCAAAAAAAAAAAAAAAAAAAAAAGGATGAAACTT
TTATTTTTTTTTTCAGTAAAAATCTCTGAATTTTTGTAGGTTTATGCCTTAAGCCCTTTCTAGGAAAAGTTAGTTTATT
AGGAAGCAGGTAATGGCTCTTTTACCTCAGGAATTTTCATTTCCTTTCACATTTTAGGTCACACGCTCGCAGATGTCAG
CCATATATCCAACATAAATGGCAGCTGTGTAAATGAAACAATGTTTGGTTCAACTACTAGCAAACTTGAATGTTCCCTT
GAAAACCTAGAAAATCTGGACATCAGTATGTTTCAAGCTCCTGAAGACTTATTAGATGGCTGTCGGGTATGTCTTTATT
ATGTAACAAAGTCAGAAGGACAAATAAGAGACCTGAGTTTAGGGCTAGCTTGGGCTACACAGGTCCAGACATCCATACA
CCCTCAGCCTGACTCCAAAGACCCCTCACAGATGGACTTGCTCCTTATGTGCGTAAGCAAACCTTGCACTAAATTAGGA
AGCTGACACTTGGCACACACACGCTGTGCCTGTGGGTCTGTCTGGGCTGTAGCTTCATCCCTTCTATTGCTGAGTTCTT
AAGTCTGCTCTGAAACCTTACCGCCTACTTTACAGATGTTCATCCTTATCATTTGTTTACAATACATTAATGACGGTGA
TCATTTCATGTTACATTATGTTATTTAAAATATAACTTAATCCCCATGAGTAAAGTGGTTATTATCTTGAAGCTAATAC
TTAGATGATTAAAATCCATTTTTAGGGCACTTAGGCTAACGTGCTTGTTGCTAAGCCTGACAGCCTGTGCTCAGTCCCT
AGCACCCCATGTGGTCGAAGGAATGAACGTAATCCTGTCCTGTGACATGTACTTGCGCCACGCCCACACGTGTATATGT
ACAAGACTTAAATACAATAATGTGCTTTCTCAGAGTCCTAGACAGATAATACTGTGGTCTTAAAGTCTGCCTTCTTAAT
TGTGTACAAGACCTTGGGTTCAGTGCTCAGAAATAAAAAGAGAAAAAGATTAGATTTGTAAGATTATTTTCAGTGTGTG
CATGGAAGCTCCGTGATGGAAGTGTCGCGTATTTAGACCCACACACCCCACACAGTGCTAGGAGGGGAGTGAGAAGCTA
CACATTTTTCCTCAGAGTAGGCAGTTAGGTTTATTGTTAAGGCCACATATCTGTGATCGCTCCTAAGATTTATCAACTT
ACTGGTGGGATAGAACGACACTTTGTGTAATCTTTTTTCTTTGATCTTGGGACAGACTAGTTCTAGATGTAACTTTGAA
TTTTTTTTTTTCTATGTAGGAAGTATTTGATTCCTAATTTTTGCTAGGATGTATTCTAGTAATGGTGATAGTTTGGGAC
ATGTGGAAATAGATTGTCCTTTTTGAAATTTTTTAAAGACTTGAAATTTTTAAAAGACTTGAAAAGTAATGTCGGCTAT
CCCTCACATCTACAACCCCTGTACACATGTTTCTTCAAAGCATTGGGCTCAGCTCCTGGATTTGTTGTTTTGGTTAATT
CAAGTCGAAAAGAAATTTCAGGAAAGATAACTATGTAGGAAGGAGTTGGCTCCGAGCCTTGAGGAGTTTGCTCAGTTCA
TATGGTTGGTCAGTACCAACTGAAAACATGCAAGCCTGGGTGCTCCTGAGTTTACAGTGAACTGCAGTGTTTCTCTGTA
ATTATTCATCTACAGGCAGTCACTCCTTTACAATTTAAGGAGCTATTTGTATGTTAGAAATAGCTCTGCCACCCACCAA
ACCTGATGACCTGAGTTCCATTCCTGGGACCATAAGGTAGGAGAGAACTGAGTCCCTCATGTTTTCCTCTGGCCTCCAT
ATATGTATTCTGACACCTGTGCATGTGCACACATATGTACACACACACACACATATACACACAGTGAAAATCTTATAGC
AAGCTGTGCACCATCATTTAGAAGATGTTGCAGTTTGTCCTGTAATTCATCTGCGGCCTCAACCCTCCTTCAGGTTTTT
ACTGAGAGATGACAGTGCTGTGGTTAAAATACCAGTTTTAGGCTGAGGAGATGGGTGAGTGGGGCAGGCACTTGCTGTG
AAAACATGGGGGCTGAAATTTGATTCCCAGCACCTATGTAAAAGCTGTGAGTGATGGTGTCCAGCAGTGGGGGGTGGAG
GGGAGCTAGCTGCTAGTGTGTGTGCACACATTAGATGTAGACTATAGTTGACTCCATGTGTATCCCAAAATGTACATGA
AGTATCTTGTTTACACACAGTGGATACAGTTTTCCTTTAAATTTGTTTCTGTTGAGGAAAAAAATAACTTGAGTATGTT
AATCCAAAGCAAATGTTTTCTTTGCAGATATATCTTTGTGGTTTTAGTGGCAGAAAGCTGGATAAGCTGAGAAGGCTTA
TTAACAGTGGAGGTGGAGTTCGATTTAATCAACTAAATGAAGATGTAACTCATGTAATTGTGGGAGATTATGATGACGA
CGTGAGGCAGTTCTGGAGCAAATCATCTCACAGGTCCTGTCAGTCTTCAGCTCAGAGCACTTGGTACAGGGATGGTTTG
GGTTAGCTTTGCATGTCTGTGCGTGGAGCTGTGGGAATGATGGCCAGTGGAGTGACGATCTTCTACTCGGGAGCCTAAC
TCTGCTCTTTTTATAGGCCTCACGTAGTGGGAGCAAAGTGGTTGCTGGAGTGTTTTACTAAAGGATATATACTTCCTGA
AGAGTCCTATATCCACACTAACTACCAGCCAGCTGGAATTGCAGTTTCCGATCAGCCTGGAAACCAGACAGCTGTCTTA
GACAAGAGCGGCTCATTCTCCAAGAGCGCCCTTGTTCCTGCTGAGAGGCTTCAGCAGGCCGACGAGGATCTGCTTGCTC
AGTATGGAAATGACGACTCCACGATGGGTGAGGAAGAGTTGATTAGCTTTTGTAGTTAAGAAGCAATAGATAATGCATG
ATAGCTGCCATATTTTTATGAACATTTTATTTTCAAAAACTTTTAGATTTGTAACTTTGTAACCAGGCAAGGCCATCAG
GCTCCAAATGATTGAATCATGCTCTCAGTAGCTCTATGTTGAGTATGAACTGTTTTTGTTTGGGGTGTGTGTTTGTGTG
TGTGTTGGTGCCAGAGGTCAGAGGTGAGACCCTCATGGAGCTAGAGTTACAGGCATTTGTGTCTAATGTGGGTGCTAGG
AATAGAACTCGCGTACTTTGTAAGAACTGTGCATCCTTTTAACCTCTGAGCTATCTCTCTGGCACCTCTGTGTGAATTG
TAATGGTAGAAAACAAAGGCCCTCTGACTGGGCTGTCGGAAGTCTTTCCTGCATGCAATACTGTTCCTGGTACTGCTGT
CTCAGCGTATAGTTTCATCGGTGTAGTAAGGTAAATTACCAACAAAGAGTTAGAACTAACCTTTTTAGAGTGTATTATT
TGATACATCAAAAGATAGGGTTTTGTGGTGACTGTCACGTACTGTTTGTTTATGAAAATAGAGCATACCCCTGTGCCAA
GAGCATTCCTGTGAAGTCCTCCTGTTTTTCGCTTTCTAGTTTGTGTCCTGTGTAGGTTTATAGATGAGCCTTTGGCCTT
CGTGGCTAGGTTGTTTAGTTGTTTACATTGCTTATTGCAATGCTGAGGCTGACTCAGGCGTAGGCTCTATCACTGAGCT
GTTACTCTTTTTATTTTTAATTTTAAGGCTTAGTCTGCTCTTGGACTTCCTCCGTGAGGCAGGTAGATTTTGAACTTGG
AGTCTTTCTGCCTCAGCTTTCTGAGTAGTTGGTTTTTCAGGCCTCTTCTACTAGGGCTGGCTTCAATTTACATAAAGTA
TATGTACTTTGTAGTTGTAAAGACATAGTTTTCAGTTCTTTTTTTTTTAAGATTTTATTTCTTTTAAAAAAAGATTTTA
TTTATTTTACGTATATGAGTACTATAACTGTACAGATGGTTGTGAGCCTTCATGTGGTTATAGGAATTGAATTTAGGAC
TTATGTTCGCTCAGTTCAACCCTGCTTGCTCTGGCCCAAAGATTTATTATTATATCTAAGTACACTGTAGCTGACAGCT
GACTTCAGATGCACCAGAAGAGGGCATCAGATCTCATTACGGATGGTTGTGAGCCACCATATGGTTGCTGGGATTTGAA
CTCAGGATTTTTGGAAGAGCAGTTAGTAGTGCTCTTACATACTGAGCCATCTCATCAGTCCCCAGTTTTCAGTTCTTTT
TGTATGTGTGTGGTTTTTGTTTTTTGTTTTTTTTTGTTGTTGTTGTTTTTCGAGACAGGGTTTCTCTGTGTAACCCTGG
CTATCCTAGAACTCACTTTGTAGACCAGGCTGGCCTTGAACTCAGAAATCCATTTACCTCTGCCTCCCAAGTGCTGGGA
TTAAAGGTGTGTGCCACCACTGCCCAGCCAGTTTTCAGTTCTTATTGAATTTTAATTGTTTAATTGTGGAAAACAAATT
TTTTTATTATTTTTCTAAATCTATTCAGTGGTACACATTTGAGTTTCAGCACTTGCGAAGCAGAAAGAGGCAGAAGGAG
CTTGAGGCCATCCTGGGCTACATAACAAGCTGCAGACCAGCCAGGACTAGAGAGACCGTGTCTCAAAAACAAAACAAAG
AAACAAAACAAAGCATGTTTACTAGACGGTGATTTTTCAGGAAATTTGGTTTTCTACTCTGGTTTCATACTTAGAATAG
ACATGACAGTTGTTCTCTGATTGACGATGGTGATACTTAGCATTTGGGATAAACCTGAAGATGGAATGAATACAGAGTC
TCCTTTCACTGTATATTTAAACCTAAGTTTTATAAGTATTAAAATTATCAGACTCTTACATTCATGTCTTTGCTAGAAA
ATCACTGTGTAGCTTTGGTTGATTTGGATATTGCTGTGTAAACCAATTTGGCCTTGAACTGACAGAGATCCACCTGCTT
CTGTTTCCCAAGTATTGGGATTAAACCTGTGCATCACCACACCCTGTGCAAATAAACTCTTTTTTTTCTTTCTATTATT
CACTGTACATCCCAATATCCATCCCTTTCCTCCCAGTCCCCTCCATAGCCCCTATTCCCCGTCCCCCATTCCCCCTCTC
CTGTGAGATGGGGGACCCCCACCCTCCAATCATCAATCACCCCATCATGGCACATCAACTCTCTGCTGCACTTGGTGCG
TCTTCTCCCACTGAGGCCAGACAAGGCAACCCAGCTAGGGGGATGGGATCCACAGACAAGCAACAGATTCAGGACATCC
CTGACTCCAGTTGTTAGGGGACCTGCATGAAGATCAAATAGCACATCTACTACATATGTTTGGGGAAGCCTGGGTCTAG
CCCATGTTGGTGGATTTTTGGCTGGTGGGAAATAAACTTTGTTTTGTTTTTTCTCAAGACAGGGGGAAATAAACTCTTA
AGTCTAGTAATTGTCAAATAGAAAACTTGCCTTTTTCTTTTCCAGTTGAAGCTAAGCTATCTGAAGCTTTAGAGCCTGA
AGTCGGGCCTTGCCCTGGTTCTGCTCACCGTGAGCCCTGTGATGATTCCACTCACATTTCTGTGCAAGAGGAAAACAAG
TCGTCTGTCAGTCATTGCATCCTTGATGACTCTACAGTTCGTGAAGAAGGCTTATTTAGCCAGAAGAGTTTCCTTGTTT
TGGGTTTTAGTGTTGAAAATAAATGTAATATTGTAGACATCATAAGAGAGCATGCTGGCAAGATTGTGTCCCTTCCCAG
CAGAATTGTTGCTGACTATGCTGTGGTTCCACTGCTGGGATGTGAGGTAGACGTGACGGTGGGAGAAGTTGTTACAAAC
ACGTGGCTGGTAAGAAGCTGTGGCAGTCGGAACCCTGTTTTGAAGGAGTATTGGCTGCCAGAGTCTTTATGTTTACTTT
TGAGCCTGTTCTGTTTATCTAAATTGGCATTTGGTTTGGGCAACGGCAATTTTAAAACATTAGCCTGGGAGAGCAGCAG
GATGGCCCAGTGGGTAGAGGCCCGAGCTGTGGGAGCCTGAGCACCCGAGCCTGAGCCCTAGACCCTGGTGGGAGAGAGA
ACTCCCACGAGTCCTCTGACCTCCGTGCTGCAGACACTTTCTCTCCCTCCTTTTCCTTTTGCTGTGCCCCCTCAAATGC
ACCGGCTATACTTCTGGCCTCAAACTCAGAAATCTGCCTGCCTCTGCCTGAGTGCTGGGGATTAAAGGCGTACACCACC
ACGCCCAGCCCAAACCTGTATTTCTTGATCCTAATACTAACATCAAGAAGTTGCTATCACAGGGCTAAGGCACCAGTAT
TCTTTAAAGTAACTTGCAATTGCTGTATTAATCCATCTTACTGATTTTGATATGCAAATGTGAAGGATTGGTATCTGTA
TTTTTCTTCAAATATCATTTGGAATATGTCAAGAATGAGCATACTCAAGTCTATGTAAAATTCCTTTTTCCTTAAACTA
AAATTGCTACTATTATAACTTGAGCTTCTCTTAACCACTTGTTTCTGATAGTAGCTTTATTGCTTTTGTTTTCAAAGGT
TACGTGTATAGACAACCAGACATTAGTTGATCCCAAGTCGAATCCCCTCTTCACACCAGTCTCAGTAATGTCGGGAGTG
ACTCCTTTAGAGGACTGTGTGATTTCCTTCAGTCAGTGTGTGGGAGCAGAAAGAGATTCCTTGGTATTCTTAGCAAATC
ACCTTGGAGCAAGGTATGTAATATTTTCAGTATATCTCCTGCCTGTGAGTGAGTGTGCATGCACACACACACACACACA
CACACACACACACACACACACACACACACACACACACACACACACACACAGTGTTAGGCAATAAGCGGAGGGCCTTGTA
CATGCTAAGCAAGGGCTCAACTATGGCAACCCTAACTGCATCAAGTTTTGTTTGTTTTGGCTTACTGGAAAGTGTATAA
TTACATTTGCCTAGGATTTTGTTTGTATATGTGGTTGATGGGTAAATACTGGGGCATACACAGCTAGGTGGATATCTGG
CAAACTTGCAAGGCTGTGGGCAGGCATTGTTTTTTTTTTTTTCCCCCATTGGCTTAGTCACCATGAAAGGCTGTTACTA
AATTGTGCTTCTCTAAGCACTGTATGTGCCCTTCAGTGCCTCCTCCTCTTCCTTCTCTTCCCCCCTTCTTCCCCCCACC
TCCTTTCTCTTTCTTATTTTAAGTGTATATCTATTGCTTGTTTTTATCGAGAAATACAAATGGAGCACTTTTTTTTTTT
TTTTTTTTTTTTTTGGTTTTTCGAGACAGGGTTTCTCTGTGTAGCCCTGGCTGTCCTGGAACTCACTTTGTAGACCAGG
CTGGCCTCAAACTCAGAAATCCGTCTGCCTCTGCCTCCCGAGTGCTGGGATTAAAGGCATGCACCACCACACCCGGGGC
AAATGGAGCACTTTGCAAGGAATGCAAGGAACACTTTGGAAATAAAGGAATTAAAAGTTTCCTATAAAATCCTCATTGG
TCATTACGTAGTAGCATTTGAAAAAGAGTAGAAATCAGCTGTATCAATGCATTTCCATTAACCAACCTCATATTTAATG
GGGATATTTTTAGGTTAAGAGCATAATGAGCATAATGCTTTTAAGCAACACTCTGAACATTCTGAGGGGTAGAGTCACA
GCAGGGCTTCTCTGTGTAACCCTCTCTCCGTAGACCAGTTTGGCCTTGATCCAACTCAGGTCGGCCGCTCTTGCCTCTA
GAGTCCTGGGATTAAAGGCGTGCACTGCCACCACCCAGAGTATGTTTCTCTGACATTAACCATGCTATTATTTTTTTAA
AATGAGCTAATTGTGTGTTCATTTGCTTTATTTCCATGTAAAATTTTAGTGTTCAAGAATTCTTTGTTCGCAAAGCCAA
TGCAAAGAAAGGCATGCTCGCCAGCACACATCTTATAGTGAAGGAACCGACTGGTTCCAAATACGAAGCTGCAAAGAAA
TGGAGTTTGCCGGCAGTTAACATTTCATGGCTCTTAGAAACTGCGAGAATCGGGAAGAGAGCAGATGAAAACCATTTTC
TGGTTGACAACGCACCTAAACAAGGTTAGAAGTCCTTGTTTTTTTTTTATGTATTTTACAACTTGATGGTTTCTGAAAT
AGGGATGTTCCAGTACTTGCTTTAAAACATTTGTATGACCCTAACCTCAGTCAGTGGTGCTTACTTCAGAACCCCTGAG
TGAAACACGGAAAGCAGATCAATGAAGAAGCGCATCAGGGTCAACCCTCGATTAGTCCTCAAGGCGAGTGACGAGAAGG
TGACCCCCGAATGGCTGTTAGAAGCAGTTTTTATACTTTTTAGTGGCACAGGTTACATCAGCAAGGTAACAGTTCATAC
TTATTGCCTAAGCATATTGACCTTAAAATCTATTGGCTAGCAAGCATGACACACATTTGAACTCTACCTAGGACTTTCC
AGGGGGTCCGATGACTATGAGTCAGTACGTTCTGCTGTTTTTCTGAGAATTTTTTTACTCCAGGATGTTCCTGTGGGTG
AATGGAACTTGGCCTAGCTTTGACCCCAGGTGAGAGGGGGAAGCTGTAAGGAGGGTGTGAGACTGGAAAAGCCCTTAGG
GTCCCTCACAATCTTTTATTTCTGCTATTGTTTATGGTAGGTCCTGTGAGTCTTGGTTGTTTGTCAGTGGTGCCATCTC
TCAGCTTAGTGTGTACTGTACCAATTGTTGTCAGTACGGCCTGAAAGGAAAGTGCACACACAGTCTAGTTCAGTGCTGT
GCAAGGCGTGAGCCTCTATCCCAGTTAGTGTTAATTCCTTTTATGGCTTGCGCTAGCATGCTGGGTAGCTAATGTGCTC
TTGCTGCTTATCTGGGTTTTTCGTATGTTCCCACAAGATAACTTACGACTTGGCTACTGTCCTAGTTACTTTTCTATTG
CTGTGATAAGGAACCATGGCCAAAAGCAGCTCGGGAAGGGAAGCATTTATTCCAGCTTACAGTCTGTAGTCTGTCACTC
AGGTAAGCCTGGAAACTGATGCGGAGGCCATGGACGAGTGCCGCTCACTGGCTTATTCCTTATGGCTTGCTCAGCTTGT
TTTCCTTTACCATCCAGGGACCAACTGCCCAATAACGTACTGCCCATAGTGAACTGGGCCTCCCAAATCAATCATCAAT
CAAGGAAACATGCACAGGCCAATCTGGTGGGCGTACTCTCAGTTAAGGCTCCCTTTTCCCGAGCCTGTGGCTGGTTGTC
GTAAAACCAGCCAGCACAGCTATTTTCCTTTGTCTTGAGTTTGAATGCCCCTTTCTCTGGGATAGATGGCTTCTTTGAC
TTAACAGTTATGGCCTTGTCTCTTCACTTTTCTCCTTTTTGTAGGATTCTTGTTGCTTAGAGAGGCTGGTTTGCTTTCT
TGTCTCAGTCAAACTATAAACTATAAAGGCAGGAACTGTGGAAGTCCTGTGGATTCTTTACTTTTATTGTTTGGCAATG
GGCGTGCTGTGAAGTAGTCAAAAACGTTTTGTTTAATGTTGGATAACAGTGGAGAAGGCAGAGATGGGAGGATGTGAAC
AGCCTCTGAAAGGCAGTGTTGCTGGGCAGACTGCAGAGCCCAGTCAGCATCCTGGGAGCACCTGAGAGCTGTTAGCTGC
TACCAAGGCGGCCTAGACATGTTCCGTTGCCACAGCAGACTAGCTCACTAGACTTTCCAAAATTTTTGACTACACTTAA
TTCTAAAGACATCTTTCGGGAATTTTCTTCTTACTGTTTTGACTAGGTTAATACACTGTTTTGTTTTGTGAATGGATTT
ATAATTATTTGTTTCACTTAATGTGTGTGTCTGATTCAATATATACATTCATGTACACATACATTCATGCACACATATG
TACACATATGTGTATGTGTGAGTATATTTGTGTATGTGATGTATGAGTGTGCATGTCTGACCACAGGTCAACATGGTGT
GTTTTCTCTGTCATGCTCCATCTTGTTTTTAGAGTCAGGGTCTCTCACTAAACCTGGAGCTCACTGATTAGGTGAGCTC
CAGGAATCTGCCTGCCTACACCACTGCACCCAGCTCTCCTTGGATGCTGGAGACCTGAATTCAGGTCTTCATTCTTGCA
TAGTAAGCACTTTACTCACTGGGCCATTTCTCCATTCATCACTGTATATTTTAATATATCTTCTGGAGTCAGGGGCACA
TCTCTGAAGCCACACTTGAAGAACACTAGTGAGGATGAAGCAGGGGGGTCACATACATGTGTGTCCAGCCTATGTTCTA
TGGAATGCATTCATACAAAGATGGCTTTGACTGTAGCCCAACACAAAATCATAAAATACCTGGGTTGTTTTTTGTTGTT
AAACCCTGATTGTGATCTTCTCTTTGTAGATGACAGTGTTCTGTTGCAGTGTCAGTCAACAGGTTCCACGCATCTGCAA
GGCTTTATGAGACCTGTCTCAAAACAAGTTTATCTGGTTTGTTTTTTGGAAAGTAGGAATATAACTTTATGGTTTAAAG
TGTACTGAGACAATATGTAAAATCTAACAAAATACTAAAATGTCACTTTGCCTCATATGAGAGAAAAGGCTCATATTAA
TGGCTCATTTGTTTTTTGTTTTGTTGTTTTTTTCATTTTCAATTTGTAGAACAAGTTTTAGAAACTAAGATACCAAATG
GAGTGAGTTCAAATCCAGATTTACCAGCACATCCTGACGCACACTTGGAAATTCACAGAAAAAAGGCGGTTACACCTTT
GGACATGAATCGCTTTCAGAGTAGAGCCTTCCGTGCTGTCATTTCACAGCAGAGGGGGCAGGACCCCACCTTTCCACCA
GTACGGCAGCCCCTTACTAAGGAGCCCTCTTTACACCTGGACACACCATCAAAGTTTCTGTCCAAGGACAAGCTTTTCA
AGCCATCCTTTGATGTGACGGTAAGGTTTGAAGGAAATGTTACTAGTGCTAATGGTTTTATGGTTTATAAAGGTAGAGC
CCCAAATTGTGCGTTACTACTAAAGTTTGTAATAGAAAGTCATCCAGAGATGTTGATAAATGGGCGCTCAGAGCTGAGA
TAACAGCTGTGCCTATATAATATGAAGTTAGGACTCTGGGTTTGCTCATGAACACAGTTCTAGGCAGATGACTTAACCT
CTGAATATCACTCTGAAATGAAATAGTCTAATGTAATGGTTTTGTAAGCTTTAAAATAGCAAATGTGTTGCTACCGTTT
CTGGGTGACTTAGTGTGCCTTGGACTTGCTGAAAGCCCTAGTGTTGCCTTACCTTTTCAGTCACTCCTCTTAAGGAGAC
AAGTGTTGTTTCTCTGTGTGATCTGAGCAGTGGGGCCAACACCTTATGAAATTTATTCATGAATCAAAGACTATTCAAA
TAGTCTTCTTTCAGATTCTCAAGATTCCTCCCCCCCCCCACCTTTTTTGGGGGAAGGCTTTTGGCTTTCTAAGTAGCTG
TTGAAGTAAGTCATTGCCTGAGATTTCTGTATCCTTGAGCCCACTTACTTTTGATAGTTTGCTTATAGGATGTTAAGAA
ATGACATTTTAGCTGCACCTAGTAGTGTTTCCTATAATCCCAGTAACTCGGAGGCCAAGGTGGAAGATCATAAATTCAA
GACTTGTCTAGGTTACACAGTGAGTTCAAGGCCTGCCTCAAACAAAAAGTACTTTTGTTTTTGTTTTGCCTAGAGTTTT
TTGTTTGTTTGTTTGTTTTGTTTTGTTTTTTTCTGAGATAGCTTTTCTCTGTGTAGCCCTGGCTGTCCTGGAACTTGCT
CTGTAGACCAGGCTGGCCTTAAACTCAGATCTGCCTGCCTCTGCCTCCCTAGTGCTGGTATTAAAGGCTTGTGTCAGCA
CTGTCCAGCAATTCTTTGCTTTAAAAAAAAAAAAAAAGAGTTGTGGAGGTAGGTCTCTAATAGAATAGTGGCCTTGTGG
ACATGAGAAGACTATGGGTTTCATCCATAAGAAATCCAGGTGCATTTTAGACATTTTTACTACTGTGTTTAGGGGCAGA
TTATTCCGAAAATAAATATTGATATCTAGTACCATTTTATTCTAGGCTGATGTATATCCCTAATCTGAAAATCAAAATG
GCTCCTTTAGTATTCTACAGTGGTCCTGCAGTTTTAAATCCCTTCCTCTGATTAGTGCTCTGCCCCATGAGCCTTCAGC
AGAGGAAAGGCACTGTCTTAGTGCTATGACTTCTGGAGTCAGGATGACCATGCTTCATGCTGCCTTCTGTCTGACACGT
GGGAACTCCCTTGAGCGTCACCATTCTTGTCCTGGGATCATGTTAAGTCACGGAATGAGTCCCAACAACTTGTACAGTT
CTCAGACCCCTTAAGTAGTAAACACAGAAAAGGGATGCACTGCTAAGTGAAACTGCAACCAACATTTGGGCAGCCACTG
TCCGAGCTCAGATAGGACTCATTCTGTGTGCCCTTCCTCATGCTCGGTATTTCTGTTTCTCCTCCCCACCACTTGTCAG
AGTTGTGTTGTGCTTGGTTTTGGCACTTACAGAGGCAGTGTTAGGAAGTCTCTTGCATCTGTTTTGTTGGATTTGTTTA
GCCTCTATTGTGGAGATTTGCCCATGTTCTATATAACTGTAGTTAATCATTTATCATAAGATGGTATTTTTTCTATCTG
AGGTAGTGTTTGCTATATATCCAGTATATTTATATGAAAACTGTAATTTCTGATTCCCAATATGTGCTTATTTACCCAT
CCTGTATATTAAGTGATGTTTGCAAGTTTCTTGTGTTGATAATTCTTGTTTCGGTTTTTGGTTTTGGTTTTTGATTTCT
CTGTGTAGCCCTCACACTGTAGACCAGGCTGGTCTTGAACTCTCGGATTTACCTGTCTCTACCTCCCAAGTTCTGGGAT
TAAAGCCTGTTCCACCATGCCCAGCAGTGTTGGTAATTCTTCTTCTGAGATTTCACATGGTTTTGTTTTTTGTAAGTAG
TCGCTTTGTTAGTTGGCACACTGATGTGTGACTTGTATTAATTAGAATATAAAATGCAACTTTTGATTTTATAAAAGAT
CCTGTCTTGTTTAATGCTTTCTGGCTTGAGGGCTGTCTGCTGTGGTTTTAGGATTTCAGTGTGCTGTATTTATCTCAGT
TCTGTCATGTATATAGTAACTTACACTTGCTTCATATACCTTTATATTATGTTTGCTTGATGTTCTTTTTTCCTTGTTT
CCTTTTTGTATGTAGAATAGAACCCCTGCTCCACTTTTGAGACAAGGTCTCCTATATAATGCAGACTAACCTTGAATTC
TGAGATCTACCTGCCTCTGCCTGTTGATGACTGGAGTTAAAGGCATGGACCTCCATGCCTAGCTTTTTCTCTTTTCTTT
CCTCTCCTCTCCTCTCCTTCCTTTTTTTTTTTTTTTTTTTTTTTTTTTTTTTTTTAAGATTTTAAAACAGTTTTACTGT
ATAGATAGCCCTGATTGGCTTGGTACATAGAGCCAGGTATTCATCACTGTTGTGTATGATGCTGGAGAATATGCCTAGG
GCTTTGTGCATGTAGTACTCCCAGCCGAGCTGTATCCCAGTACAAACCATAAACATGTGTTGATCATTTGCCATTGTGG
TTACTAGCTAGTAAATTCCCTACTCTTTGCTTAGTTTCAGCAAAGAGTAATTGAGGCTTTTCCCTTCTCTCCTTTCTAG
CCTCTCCGAATCTTGGGAGAGTTAGCATTAAAGGTGTATGGGATTGTCAAAGCCTAGAGTTGACATGACAGGTAGTGCA
TCCTCTCCATATTAACTGTCCACTTCTCCTGCTGGCATATTTTACTTAGTTTGGATATTATAAAATTTATGCAACTGCA
TTCCTGAACATATTCATTCATATTATTTCTGATCTTTCCTTCTTGAGGTGTGTCACTTTTTGTTAATCCACATCAACAG
TAGAAGGATCTGTTCTCAAGAACTTTGTTTCTCAAGAACACTTCTCTCTTTGTGATCATGCATTCCTTGGTATCATGAG
GCTGGATGAAAGAAAAGAATGGCTTCCTCCATTGTAGTCTGCAGAAACCCCAGATGAGAGGCATCTATCTATCTGTCGT
AGAACTTTCAGCACCCATTGTCATGAAGGGATATTAGAACTGTCCTCTAAGGTGCCCAGAACACTTACGCCTGTCTCTA
AAGGTGTCAGATGCTTGCCCACTTTCAGCGTTAAGTGCAGTCCTGGTTAGCTCTGAGCTCTGTGGACTGCTTGGGAAGT
AGCCAGACCCTGCATGGAGCTCTCTGCGTCCAGTTCTTAGCCTCCCCTCCCTTTGAGCCTATATTTTATATCTTGTACA
TTTTGTTGGTTCTTGCCTCAGTCTGACTTTTTACCTAATCTTAAAATTAATTTCTTATACCCATCCATTCTTTCTGGTT
TCCTTCTGTGTTCACTTTAGTTTGGGGTATTTTATGTAATTGCAATTACAATTCTTAAGTGTGGTATGTATGCACTGCT
GAAAAGGTAAGTATGCCTAATGGTTTTGTTTATCTGTTTTAAAATCTATTTTTTATGTGCATGGTTCTTTTGCCTACAC
ATATGTTTATGCATCACCTGCGTGCCTGGTGAACATGAAAACCAGAGGGTGTCAGATCCCATGGAACTGGTGATACATA
TAGTTGTGAGCCACCACGTGAGAGCAGGGGATTGATTCCCTGGGTCCTCTAGAAGAGCTCTCGCAGTACTCTTAACTGC
TCAGCCAACTCTCCAGCTCCTTACTTAATGCTTGGTGAAGAATATGAAATTCTTTTAACTCTAGTATAAAAATAAAAAT
TAAAGTTCATGCTTTTTATTTCAGAGAAAAAGAGGTCAAAGATGTAATCTCTTTTTTCCTTTCTTCACTACTTTGTCTC
TTTCCTCTGTGTCCCCCTCTTCCTCTCCTCATTTTTTCCTTTTTCCTGTTCTTAAACACAGGATGCACTTGCAGCCCTG
GAAACCCCAAATGCTGCCAGCCAGAAGCGGAAACTAAGCTCACCACTCTCTGAAGTCATCGTCAGAAACCTGACAGTTG
CTTTGGCAAATAGTTCCCGAAACACTGACAGTCATTCTGCCAGCCCTCAGCTGAAGGGTGCCCATTTGGAGGAGGTAGA
CATGGTTTTCTTATTAAATGGAAGGTGTCCCTAATGCATGTCAAAGAACTGTGGCTGGTCTGTTTCTTTATTCTCCATG
TGCAGGAAGAGACTCGGAAACCACTTGACAGCGTTGTGGTGTGTGTGAGTAAGAAGCTCAGTAAGAAGCAGAGTGAGCT
CAATGGGGTCGCAGCATCACTGGGAGCAGAGTACAGGTAGGGACAGCTCTGTGTTGGCGGCGCCAGCCACAGCTCAGAC
AGCACAAAAAGCCTGTATTCATTTATAAGGAAGACTTGGCTTTGCCTGCTTGTGTGCATTATCTAATAAAACCTCTCCG
ATTAATTAAAACTGTCCTCTTAGGTACCCAGAACACTTAAGCCTGTCTATAAAGGTGTCAGATGCTTGCCCACTTTTAG
CCTCCAGTGCAGTCCTGGTTATGGACTGGTCATGGGTTTATAGTCAGCACATCTGGGACTGACTGCATTTATTATCAAT
CTTAGTGATTTCCTGTGAGTAGTACTGACTGACCACTGTAGCAAACCTTCCTAGATTCCCCTGACTTCCACACTCCATT
AAAGACATTTTAGTTTGCTCTAAATAAAAATTTATTTGTGTGTGTGTGTGTGTATGTTGGCATGGTGGTACACTCCATT
AATTCCAGCGCTTGGGAGGCAGAGGCAGGAGGATCTCTGAGGGTCTACATAGTTCCAGGATAGGCAGGGCTACAGAGTG
AGAGACCCTGTCTCTAATGTTTTCTTAAGAGACGTAGTCTCATGCTGTGGTAGATTTCTTGCCTAGTGTACTCAGAGGC
CTGAGCTCAGTTCTCAACATAGATAGTTTTGTGATAGGGACTAACTTGATAGAGTGAATAAACAGGGTGGTGGCCAGTG
ACTGGACAGTGGATTTGAATGCCTTAAGTTTTATTAACTTGAAAATACTTTTTATTGTAATTAGAAAGTGATATACATT
ATAAGACAAAATGGAAGGATTCTTAAAAATATAAAGAAGATAGTAGTATTTTAAAAGTATTCTTCTGGCATCTACACTG
TATAAATGCTGTGTAAATGGAGTCGCTAGCATTTGTCAGGATACAAAATCTGCCATTTACTACTCTTGCTTATTCTCTA
GTTCTTTGAGTTCTGTGTGTCAGAACATTCTGTTCAGAGTAGACTGCTGTTTCCCTTAGTTATTCCTCTTGCATATTTG
GTTGTTTCAGGTGGAGTTTCGATGAGACTGTTACTCATTTCATTTACCAGGGTCGGGCAAATGACAGTAATCGAGAATA
TAAATCTGCAAAGGAAAGAGGGGTGCACATCGTGTCCGAGCATTGGCTATTAGAGGTAAGCAATGCTTTTCCCTCTCTT
CCTTGTTGGTTGAAAATACACAAATATTTCAGAGCACAAACCAGTTTCATTGAATGCAAATAAATTTGCTAATCTTGGC
TAACTGTGTGGTGGCATGTCGTGCCAATACCTGCCCATTTCATGGCTGGGACATGAAAGAGGTCGTGAAAGGGTTGGGA
TTGGACTGCTGTCCAGGGCACACCCCGAGGGCCTGTTGGATGCCATGGGAACTGTGCTTGTAACATAAGGCCTTGGGAC
ACTCAAAATCTGAGCTATATGTAGGTCATGCTTGGGGCATTGTATCGTTTTAGCATTAGAACTTAAATTGTTTTAAGAT
TAATTGGTTGATTGGTTGATTGATTATTTTTGTATTTTTATGCACATGAGTGCTCTATCTACTTGTACACTCGAGTGCC
TAGAAGAGGGCATCAGACCCCATTATAGATGGTTGTGAGCCACCACGCGTGATAGATGGGGAATTGAACTCAGGACCTC
TGGAAGAACGTACATCTCTCCAGCCCTAGAACTTAAAATTTTTATTGCTTGAATATTTACCAAGTTTTAAATGAATACT
TATATCATGATTCCTTTCCCTCATTGACTATTAGATGAAGAGTAGCTAAGGGGCTGGAGAGATGGCTCAGTGGTTAAGA
GCACTGACTGCTCTTCCAAAGGTCCTGAGTTCAATTCCCAGCAACCACATGGTGGCTCAAAACCATCTGTAATGAGATC
TGATGCCCTCTTCTGGTGTGTCTGAAGACAGCTACAGTGTACTTACATATAATAAATAAGTCTGAGAGGGAGAGGGAGG
GAGAGATTGAGAAAGAGAGAAGCTGAAAGAAAGGGTGTGGAAGGTAAAGGCTCTCACCACCAAGCTTGACTATTTGAAT
TTATTTCCTGGGACCCATACAGTGGGAGGAGATAACCAACTCCTACAAGGTCTGCCCCCCATATAAATAGATAGAAATT
AATTTAAATCATATAAAAACAAAAGATGTTTTTGAGGGTAGTTGCTAGCATCCTCATACACTATAGCCATGCTTTTCTT
GGTGCTCTCTGGAATGGCTGATACAGAGGCTAAATGCAGAGGCAGGATTAGAGTTTATACTTTAATAGACAGTAGATTG
TAAAAGTGGGGATAACTTGTTCCACTTTGTTTAATTTACTGTGTGTGCATAGGTGTGCTCAGAGGGCCTTTTACTCTTT
TTACAGTTTTAATATGGTTTCAGTGATTAAACTCATGTCACCAACTTAATAGGACAAGGACATTTACTGGCTAAGTCAT
TTCCTGGGTCCCAATTGGTTATTTACATGCTAGAGTTTTCCACTGTAAATTAATTTCCTTCCTTCCTTCCTTCCTTCCT
TCTTCCTTCCTTCCTTCCTTCCTTCCTTTCTTTCTTTCTTTCTTTCTTTCAAAGATTTATTTATTTTTTATATGTAAAT
ACACTGTAGCTGTCTTCAGACACTCCAGAAGAGGGCATCAGATTTCGTTACGGATGGTTGTGAGCCACCATGTGGTTGC
TGGGATTTGAACTCGGGACCTTTGGAAGAGCAGTCGGCACTCTTAACCACTGAGCCATCTCACCAGCCCCTGTAAACTT
CTTTCTAAAGTGTACACTTGTGTTTCAGTGTGCTCAAGAGTATAAGCATCTTCCTGAATCTCTTTATCCACACACTTAC
AATCCCAAAATGAGTTTGGACATCAACACAGTGCAAGATGGGCGGCTCTGTAACAGCCGGGCACCCTTGGCAGTTTCTG
CATCTAAGGATGATGGGGTAGGTGGTGTGGACAGTGAGCCCCTCCCAAGCTGCATGTAGATGTCTGTGAAACTTTATGC
AGTGAACTTCGTTTGAGAAGTTGTCATTTTTCTCTTTGAATAGCCAGATCATCTGTCTGTAGAAGGAAATGAGACAAAC
ACTATGGGCACAAATGACAAAGAGTCACCTCTGTTGAATGGGAGTGGAAGAGATGACTGTAAAGGAGGTAAGACACTTG
TCACTTTTTACCCCTTACTCTCACGCCATGGCCTCCCAAGACAGGGTTTCTCTGAGTGGTTCTGGAACTGACTCTGTAG
ACCAGGCTGGCCTCAAACCCAGAGATCTGCCTTCTTCTTTCCAAGTGCTGGGATTAAAGGCCTGCGCCACCACACTCAA
GTGCACTTTTTACTTTTGACAACGTTTGTTTTGACTTACAGTCCCAGCTGTATTTTACCACTAATAGAAGAACTGGGAT
GGCCTTCCAGCTTTGCAGCCAAACACTGACTTCTCTCCACAAAACCCTAGAGAGGTGTCTTCTTCCAGTCTGAGTCAGC
TGCTCATTCTCCTGCAGCTTCGCATCAATGAGGAGCTGTGCCCACTGCAGGAGCACTTGGAATTTCTCTTTGCAGTCAC
AGCTTGACTGTTCAGCAGAAGAGCCTTTTGACCTAACTTGACTTTCATGCATGCCTTTGTCACTTGCCTTAGTCACTGC
TAGCTTTACTTTAGAGCTAGAGAGAGGAGTGTGACTCTTCTCTTCACTTGAACACTAAGCCACTGTAGGATGTTAATTG
AGCTAATTTTAGAACTGTTGTTTCTTAGGGATTGCGGAAGTCCTTGGAAGGAAAGAGAGGAGGAACAACCAGTCAGCAT
ATACAGCTTTTATCTGTAGCTTTTGTGTAGATATGGTTCACACAGAATCATTTGGAAGAGTTGGACAATTGTTAATATA
TGGTTGCTACAGCTCTCCATTTGCAAAAAAAAAAAAAGAAAGAAAAAAAAGCAATGTTTTAGTGTAGCTGGAGCTGAGA
AATATATAGTAAAGGGTACATTTTTAGCTCAAACAGCCTTAGAAAATGAAAGCCCAAGCTTGATTGGTTTGGACTCTGA
TGAAGACCTGGCTACATCCTATCATGTCACTATCAGAAAGGGAAAGAGTGCAAGTGGGTGTCACCACATGAAAGACTGC
AAGCAAGTTAGACCAGGCTCACTGTTGTCATAGCAACCCACAACAAAACTAGCCTGAGTTCCCCCTGAAAACTGCATTA
CTTCTGGGGTGGTGTTGCTAATGACCCAACCCCCTTAGACTAGATCTCACTCTTAAGGAGTCTCCTTACTATACCACAC
CACACACAGGAGGGTGTGCACTCAAGCCAGAGAAGAGTGTCTGGGAAACAGGGGCTGCTCCAGTCCTGGGTTTTATTTG
CTGGTGTAGTGTGTGGCTTCTGTCCTGCTTTATTTTGCTGTTTCCTGTTCAGTGTCACTAGCAGTTATTTTGAATTTCT
TGCTATTACAGATAGTGCTGTGCTGATTACCTTACTTAGATGTGTTACTCTCTGACTTCTCCTTATTCAGCCTGCCTAC
TAGAACTTGCTTTCTGACTTAACTGTTCCATTCTGTTTATTTCAAAAATGCCTACATGGTATCTACTTCAGTGTCTATA
ATGGGAAGGAAAGACAAAGACGTTGTGGTGTGTTCTTTCAAGGATTAGTGAAAGCTCGGCTAGTGCTTTACAGCAGCAA
TCCCAGGCTTGCTGCTGAGGCAGGAGGGCATTCAAGGCCAGCATGAGCTATGTGGCAAGATCCAAAGCAAATGGACAGA
CAGCTATACATACAAATGACTGTGAATCTCTGTAGTATGCATCCATGAGAAAGGGTCAGACACGAGCAGGGAGCTCCTC
AAAGGAGCAAGTTTTCCTCCAAGGGAGTCTTCATCGCTTCAGAAACAGTCCTCATGGCCGCCTTTGTGTTTTGTGACAT
TTTAGCCTTGTATTCCTGTATAAATGTTCAGTAAATGGCAATTCTTTGTTCAGCTAGATCTAGACAAAATAGTGTAAGA
TATGATTGACAAGAGAAGTTAGTAACTGTGGTAATGCTTGCCTGTAATCCCAGAACCCTAGAGACTGAGGCAGGGATTA
CAATGGGAGGTCAGCCTGGCCACACAATCAGATCTATCTTCACACCAGCAAGGTGGAACGGGGGGGCACATCTTTCTTG
ATTGATATCGATTACTCTGTTTCTAGCTCTGACCCAGGCCTTGGAGATGAGAGAGAACTTCCAGAAGCAGCTGCAAGAG
ATCATGTCAGCCACCTGCATTGTGAAGACGCCTGCCCAGAAGACTTGCATGTCAAGAAGCAGCTGTAACAGCGCCTCTT
CAACTCCTGACAGCGCTCGCTCTGTGCGGAGCGGACGAAGCCGAGTCCTGGAGGCACTCAGGTTTGTGCGCTGTGTTGT
TTGCATAAAAGCCCCTGGTAACTTAGGTTGGTGGGTTAGGCTCTTTTTGTTGTTTTTGTTTTTCGAGATAGGGTTACTT
TGTGTAGTCCTGGCTGTCCTGGAATTCTGGAATTTGCTCTGCATACCAGGCTGGCCTTGAACTCAAAGATTCATCTGCT
TCTGCCTCCCAAATACTAGCAATAAAAGGTTTGTGCCACCACACAGGACTTTAATTTTCTTTTCTTTTCTTTCTTTCTT
TTTTTTATTTTAAATAAAGTGGAAATTAGAATATTGAGACCAGTGAAGGGGTTTGTGTTGGCGCTTTGTTTAACTCAAA
CAGTATGCATTATATCCCACTGTCATCTGGGGTTTTGTGTATTGCTGCTGAGTTTTTTGTCTTGTTGCTCAAGCAGTTG
TGAGTTCTTACTTTGAAGTATGTGATTTTAGGTGCACTTTTCCACTTGACGTAAAATCCGGGAGCAGTCAGTCCTCTCT
GTCACTTAGTGTTAGCACTAGACAGTGAGGACACAGAGTACTTCAACTTTAAACAGCTTTTTTATTATCTTGTGTTTGA
GTGTGATATGTGGGCGTGAAGACATGCATGCCATAGCACCCATGTCGAGGTCAGAGGACAGCCTGTGGATGTCACTCCA
CCCTTTCCTCACTTTTTATTTCTTACTTGAAGTGTGTGTACGTTTTTCCTCTGTGTTCTAGGAAAGGTGCCACAGTTCT
TAATGCAGTATAACTTAAGCTCATTGAAATTACTGTATTTTTCTTTGTCTTTATAGTTATAAGACAAGGTCTTATCCCA
GGCTGGCCTGCAATACTGTGGAACTAGTGTTAGCCCTAAACTCCCAATCCTCCTGCTTCAGTCTCCTGACTGCTGGAAT
TACATGTGTACCGCCACTCACCCTCTCTCTGTATTGCTGTGTAGCCAAGGTGGGCTTTAAATAAACTTGGTGTTTTTCT
ACCTCAGTTTACCAAGTGTTGGGTTACTGGGCCCACCAATAGCATTTTTTTACCTCTCTCTGTATTGCTGTGTAGCCAA
GGTGGGCTTTAAATAAACTTGGTGTTTTTCTACCTCAGTTTACCAAGTGTTGGGTTACTGGGCCCACCAATAGCATTTT
TCTACTAGAAGAAAAAATCTCTGAGGTGTAGGGTGAGAGATTTGAGTCCATGTTGACTTCATTTATATCGTACTCTGTG
TTTTCCTTGGGGTAATTTTGAATTCTGAGAAAAGTAGCTTTTCCCAACAGTGCAGGTCATGTTTTAGATATAGAACCTT
ACTCTGTGCCAGGTCGTTTGGGACTAAAAGGCTTTCCTGGTTCATTGGCTTGTTTGTTTTTAAGACAAGGTCTCACTAT
GTAGCTTGAGCTGGTCTGGAGCTCAGAGATCACCTGCCTGACTCTGCCTCTGCCTCTGCCTCCATCCACTTTGGGATGT
AGTCCTTACTGTCCTGAAACTCAATATGTGGACCAGACTGGCCTCAAATTTACAGAGACCCGCCTCCTACTGGCTACCG
AGTCACTTACTAACTGCATGTAGCTTGGTGATGGCGCAGGCCTGTAATCAAGGCCCCAGGGAGATAGCCAGGTGGTAAA
GGTGCTTCACGCCAAGCGTGATGACTTGAATTCCGTCCCTGGAATGGTGGAAGAAGAGAGCTAACTTCTGTAAGCTGTC
CTCTGGCCTCCACATAAGGTCACTGTGTCACATATGTGTATACATACACACAAAGTAAATAAATAAATGTAATGAAAAC
ATTTTTAAGGGACTAGAGAGATAGCAGTTAATGAGTGCTTGCTACTCTTGTAGAGGCCACAACTAACTGCTTGTGACTA
CAGGTCGAGGGGATCTAACAACTTCTTTTGGCCTCCCTGGGCACCCACATCTGTACATATAAACAAAATACTTGAAAAT
ATTTTAATGAATTCATAATTCTGTAATTCAGTAAATGTCTACCATAGACTGGAAATTTTGGTCACCAAGTGTACTGGCT
AGTTTTGTGTCAACTTGACACAGCTGGAGTTATCACAGAGAAAGGAACTTCAGTTGAGGAAATGCCTCCATGAAATCCA
ACTGTAAGGCATTTTCTCAATTAGTGATCAAGGGGGAAAGGCCCCTTGTGGGTGGGACCATCTCTGGGCTGGTAGTCTT
GGTTCTATAAGAGAGCAGGCTGAGCAAGCCAGGGGAAGCAAGCCAGTAAAGAACATCCCTCCATGGCCTCTGCATCAGC
TCCTGCTTCCTGACCTGCTTGAGTTCCAGTCTTGACTTTCTTTGGTGATGAACAGCAGTGTGGAAGTGTAAGCCGAATA
AACCCTTTCCTCCCCAACTTGCTTCTTGGTCATGATGTTTGTGCAGGAATAGAAACCCTGACTAAGACACCAAGGGTTG
AAAGACCCATTAGTTATATCTTCAGCAATGTCATGTTAAAAATACACTTTAAAGCTGGGTATAGTGGCACCCACGAGGC
AGAAGCAATGGATCTGTGAGTTCAAGACCAACCTGGTCTTCAATGTGAATTCCAGGACAGCCAGACCTACATAGTGAGA
CTCAATGAGATATAATTTAATTACTAAGACAGGTACGGTGGTGCATACCTACAGTCCTAGCACATGGAAGCTGCTGAGG
GGAGCTCATGGTCGTCCTTCACTACAGAGAAGCGTCTTTAAAAGAAAATGTAAATGCAATTATTAACCCACTAGCTACA
GTTCTCTAAACATCACCCTGGTTCCCTAATTTTTTAGAATTTAAAAAATACCCGGGCTTGATGGCGCACGCCTTTAATC
CCAGCACTAGGGAGGCAGAGGCAGGCAGATTTCTGAGTTCGAGGCCAGCCTGGTCTACAAAGTGAGTTTCAGGACAGCC
AGGGCTACACAGAGAAACCCTGTCTCAAAAAACAAAACAAAAAAAAATTAAAAAATATATAATTATTTTTGAAATACAG
TTATAGACATTCACTTAAATTGTCTAGTAGAAATATTTTAGAAGTAATTAACTGGGTGTATTGGTGCATGCTATGCTGT
GTAGGTTTTTGTAAACTTGGGTCATTTAAGAAGAGAAACCTCATCTGAGAAAATGTCTTTATCACTCTGGCGTGTAGGT
AAATCTGTAGAATATCTTGGCTAATGAGTGATTTAGGAGGGTCCGGCCCACTGTGAGCCCCTGGGACTTCCTTTGAGCA
AGCCATGCAAGTCAATAAGCAGTTCTCTTCTACAGTCTCTTCAGTTCCTGCCCGGACTTCTCTCAGTGATGGGCTACAG
CTGTAGGCTGTAATAAATCTCACCAGGCTGCTTTTGATCATATCTTATCACAGCAGGGGAAGCAAACCAGGATGCATGC
CTATAATCTTAGCACCTGGGAGGCAGAAGCAGGAGAAGCACCTCATTTAAAGCCATACTCTCAATAATGAGACCATATG
TCCAAAAAAAAAAAGAAAAGGAGAAGAAAGGATGTCCTCTTGATTTATTCAACAAATACTGTGTGTCTTTGCTGCTTAC
TGGTTTTCCGCACTTGGGTTCAGTGTGGCTGCTATTTAACACTGTCCCTTTTGTTCCTTAGGCAGTCTCGGCAGGCAGT
GCCTGATGTGAACACGGAGCCGTCCCAGAACGAGCAGATCATTTGGGACGACCCGACAGCAAGGGAAGAGAGAGCAAGG
CTTGCCAGCAACTTACAGTGGCCCAGCGACCCCACACAGCATTCTGAGCTTCAGGTTGAGATAAAAATGCCGGATGACT
CTCCCTCTCGAAAGCCTGTGTACCATTCAGAAATTGCTGAGCAGGGTAATAAGACAGACATTGACTTGTGCTTAAGAAT
GCTGACCTCTTAAAGACACCATGGTTCTCTGTTACATATGCGAATCAAAATGAATTTTCTAGTACTGTTTCAGTCAAAT
GAGAAATGTTTTTTGATTTTTAAATATAGAAATTTAGCTATAAGTTTCTGAAGAAGAAATTGTCATTTTTGAAGCTTCT
AGCCTCCATACAATCTAATCTGGGGCCAGAATATTATCAGCCTCTGAGCCTTACTGCTGACTTAAGTTCACCCTTTCTA
ATTCTTCTGAGCTCTAGCTGGATGGCTCAGCTTAGCTGGTCAGCTGTAAACTCCTCTCCAGAGTGACTGATTCAGTCTG
GCTTCTCTCAGCTTCTCCTGAACTGCTCTGCTTGGCCTCAGACTGTCTTTGGCAACATGTTCTAATCTTATTGCTTCTT
CTCGTTCTCTGGCTTGTTCTGTTTTCACCTGTGTTTAGCTGTTCTCTCTGCAACCTGTCCTTCTATAATAATGTCTGGT
AAAACGGCCTTTCTTCTCACTGTGCTGCTTCCTATAGCTTCCCTTTCCTCTCCTTGTTATTGGGCCTGTCCTAGTCTGT
CAGATCTTTCTCTTGATTCAGCACTTTGTCTGCCACTCAGTTAGACATGGCTTACAAACATGGGTGCTTTTTTTCTACA
ACTAACTTTACCTTCATTGCTGGGAATTAAGAGCAAGTCTGTATTCCAGCCAGAGGGATTAAAGGTGTGTGCTGAAACT
CAACCACACCACAAGTAAAAACAGGTTTTCTCAGTAAAGATCACAATCTTGGGGTTCACCGTGTGATCAAATATCCTGA
AATATTTAGGATCAGCCTTTTTTTTTTTCCCTTTCTACCCTCTTCTGTTTGTGAGGTAATGTCTGATGTTGTTTGGCTT
ATGAGCTTTGCCTGAGTTCCTTTTTTTTTCTCCCTTCACCTCGGTTAAGGTTATCTTCCTGTGCTGGATATACAGATCT
GATTTTTTTCTCCAAGAATTGCTTTCATTTTAAAAAGTTGGGTATGTTATACTTGTGAAAAGATGTTTTAGAAAGGGAT
TCACAAAACTCAATTAATAGCTGCCCTTTCAGAGCTATTTAGTCAGATGTACCTATAAGCTGGGGAAACCAACCACTCT
TTCCTCTATGTGTAGTGCTTGCTTTTGAGCCATGCAGGGTTGTACCCATGCAGGGCTCAAGCCTGAGAGTCTCCTGTCT
CAGCCTCCCAAGTTACTGGGGTTACAAACAGATGCCACCATGTCTGAGCCTTTGTATACTTTTGATTGCAGAATTCATG
GCTCATTTTATTTTGGCCAAGACAGGTGAGAACATTTCCAGAGTTTGTTCTAAGTTCCATGTGTACACTCTTACACAGT
ATTTCTTTCTCCTTACCCTTAGCCTCGTGTGTTACTCAAGCCCCAGGGCACCCAGGCTCTGAAGAACCAGAGCCTCCAG
TAGCAGAAAGGCCTTTGATCCCCGAGCCGCAAGCCCCCGCTGTAGCTTCCCCCCTGGCTAAGCCGCCTGTGGCCCCACA
ACCTGCAGACAAGGTTTGTTAACCTTCGGTATGGTGATGGCGGGCGGTTTTGTGTATTTTATGCTGGAGTAGGAAGTGT
TGGCTGCAGCAGGCTCATCACTACTGAAAAGTGCATCTCTTTGAATTACAATTTGTATACTTGGTCCCTTAGAAGACTA
GATAAAATGTCACTATAAAATAGCTATATCTGATAATAGTTTTAATGTTAATATTTTTCTAAAATAAAACTATTGTGTT
TTAGATTGAAACACAGGAGGAGACGCACAGAAAAGTTAAAAAACAGTATGTGTTCCAGATGTCGTCTCTGAATTCTCAA
GAGCGGATTGATTACTGTCGCCTGATTAAAGACTTAGGTGCCATTATTTTACTTTTTTTTTTTTTCAATTTTCTTCCAA
TATTTTACTTTAAAACATTGTTATTACAACTTATTTAGAATACAAATGCAAACATATTTCTATCATGTGTAAAGGGGAG
GGCAGTTGAGAAAAGAACATGGTTAAGTTTTGGATTTTGTTGGTTTTTTTTTTGGTTTTGTTTTTTTTTTTTGAGACAG
GGTTTCTCTGTGTAGCCCTGGCTGTCCTGGAACTCAGTCTGTAGACCAGGCTGGCCTCAAACTCAGAAATCCACCTGCC
TCTGCCTCCCAAGTGCTGGGATTAAAGACATGTGTCACCACTGTCTGGCTATGGGCAAGTTATTTAAGCCTACACATTG
AGTCTGTTCTGCCTCACAGACCCAGGTACACAGATAGGCAAAATACACATACATATAAATAATATATATATATATATTT
TTTTTAAGTCAAGGGTGGGACTGGGGGTTGTATGGCTCAGTGTGAGTTTTTTTGGGGGGGGGCATATATGAAGCCCTGA
ATTTCAACTGTATACACGGGTCAGCATTGGAAGTTTTATTTTTTATAAAACCCCAATCTACATTTCAAATCACTAGATC
TTGGAACGCTGAAGGCAGGGATGGAGGGCGATAAATTAAAGTGAATGTTACTTTTACATCTCTAGGTTGATCATTTTAT
ATAAATAATTTTTATGCACAGTTAACATTTTTCTAGAAAGAAAGTTAAGAACTAGTAGATGCCGTTTCTCTCCATACAA
GGTGGGTCGGTGATCGAGAAGCAGTGCTCTGACCCCAGCTGCACACACATGGTTGTGGGCTACCCACTGCGCAATGAGA
AGTACTTAGCTTCTATGGCAGCTGGTAAATGGGTGCTTCATCGCTCCTACCTAGACGCTTGCAAGACAGCTGGGCGCTT
TGTGCAGGTGTGTATTCATATTCCTGTGAAGGTTTGTTCTCTTGTTAATGACTTAGGAGATCCTTGGTCATCCCCTCAC
CGTGTATTTAGTTTAAATGACCACACGTGGCCAGTCAGATTACAGCTGTTCTGTGAGGATCTTTGAAACAGAACAAAGT
CTGCAAGGTCGGGCTGCTTTATTCACATCCCTTTTTATTAAAATATTTTGGTTGGCATACAAAAAAATGAGTTTTTATT
GTGGCCTGCTCACTTTTTGTTACCTAATTTATCGGTGCTTTTTTATTGAAATGATTTTCTCTTAATGAGCAGATTCAAG
ATAGCCCATTATAAGGCTTTTTCTTTGCTGTTAGCTTATCCATATTAAGCAGTTGTAACAAGCTGGGCATCACGGTAAT
ATCTACTGTGTCGTTTCCGAAGCTTCCAATCTGAGCTCCTTGGGACTTGTCTTAGTTCATACCTCCTCAGGCTGTGCTC
TCATGAAGCCAAATACTAGGGTATTTTACTAAGTTAGGTATGCTACTAAGTTGCCAAAATACAGCTGTCTGGCCAAAAG
GTTTGTTTTGGGGAAATTAGTTTCTGGTTTTAATTAAAGCCAGCTGTACTGGTTCTTCACATAAAACTGAATAAATGGA
ATGAGTTCCCAAAGTAGTGTTCTCTATCATTCGTGTCTCTGTAACGATTGAACTTTCAGCTTTGATCTTGATAGACTGC
AAGCTGCTGGGGGCCAGATACCCATGCTTAGTTAGCACAGTGCTGTGAAAGGAACCCACAAGTTCACTGGCCTGGTTTT
CTAGGAAGAAGACTACGAGTGGGGAAGTAGCTCCATACTTGATGCTCTCCCTGACGTCACTGAGCACCAGCAGAAGCTC
GCACTGGCGGCAATGAGATGGAGGAAAAGAATCCAGCAAAGTCAAGAATCCGGCATTGTTGAGGTACTATTGTGGGATT
CAGTATTGTGACTTGTTTTAAAGAGCATATGCCTGACCAAGTAGTTCTGATTGTCTTTTATTCCATAATTGATATATTT
AGTTGCACATTCTGTGTTGAACTAAAATTTCTGTTGTGCAGGGAGGCAGAGGCAGGAGAATGTTTGAATTCCAGGATAT
CTGAGGCTATGTAGAAATCCTGCCTGAGACAACAAAGTATTGTGTTACTGTAAGATAGGGTCTTCTAAAGATAACTTGT
CTTTTCAGGGTAGAGCATCAGGAGTCCTAAGCCAGGTCTGCAAATTCTCTCTAAGGAGTCTATATACTTTTGAGTAAGG
AAATTATGTCCTTTCTTTCCTAATTGCTGATTGCTAGGGTACTCCTGTAATCCTAGCACTTGGGAGGCAGGAGAAGAGC
AAGAGGTCAGAGCTAGCCTATGAAACTTTGTCCTTTTTCTTTTCTTTCTTTCTTTTTTTTAAACTAATAATGGGTTGAA
GAGGCCGCCCAGGGGTAAAGGCACCTGCGCTGCTCTTCCAGAGAACTCAGATTTAATTCCTATCATCAACATCTGTAAA
TCTACTTCCAGAGGATCCAACACCCTCTTCTGACTTCTGTAGGCCCTGCATGCAAGTGATACTTAGTATATGTAAATAT
ATGCAAAAAACCCACTGAGTTTTGTGTGTGTGTGTGTGTATAGATAGATAGATGAATGACAAGAAATTACTTGACACTA
AACAATTAAATTCATGCATTATCTCTGCATTTTGAATGTTTAGTATGTTAAAATTACATGTACTTATTTTTAAATAGGG
AGCATTTAGTGGCTGGAAAGCTATTTTACGTGTTGATCGGCCCCGAGAAGCTGGGTTCAAACGCCTTCTTCAGGCAGGA
GGAGCAAAGGTTTGTCCTGAATTAATTTACTGCTATCCTAGGGGCAGGTTTGCACCAACTTGTCCAGCAAGGTTTCTGT
GATGTCTCCCTGCCCTCTGCCTTTGAGGGGAGGAGCATCTGCTATGGAGCATGAGGGGATTAACCCTTCCTGTTTTTCA
TTAGGTGTTATCTGGTCATCCAGAACCTTTACTTAAAGATGCTACGCATCTCTTTTGTGACTTCAATAAACTGAAACCT
GATGACTGCAGAGTTTTCATCGCAGAGGCTACTGCCCAGAACATGGTCTGCTTGAAAACAGAATATATCGCTGATTATC
TCATGCTGGTTTGTCACATTCTACTCTTCCGTTTATCATTATCATTTTTATTACTTGTGTGTATATGTGGGGTGGGGAG
TTGGCACATGTGTGCTCGTCAGAGGATAGCTTAATGGAGTTGGTTCTCTCCTACCATGTAGGTTCAAACTTAGGTTGCC
AGGCTGGTAGCAAGCGCCTTTACTTGCTGAGTCATTTTGCTGGCCCCATACTTCCTTATTTCTAGAGATATGTGTATGT
TTGTAGATGAACATCCACACATTTACTCTTGGCTTGCTGCTAATAGAATTGACAGTCTGCCATTCTCTAGTCTTTCAGT
TTAGTTCCATTTCTATATTGGATGCCTGTAAACTTTTTAGCATGAAATTTAAGTATGACAGGTTTATGAAAATCTGGGT
AGCTATAGATGTTTTACAAGTTCTTTAAAATATTGGGCTATAAAGAAAGCAGGGGGCTAGAGAGTTGGCTCAGAGACAA
GAGAACTGGCTGCCATTGAAATCAGACTCAGTTCCTACCTAACAGTAACAGACCTACCGTGTGTACCTGTGTTCGTGTG
TACCTGTGTTCGTGTGTGTGCTCGTGTGTGTGTGTGTGTGTGTGTGTGTGTGTGTGTGTGTGTTCGTGTATAGAGGTGT
CTGTGGAGTTCAAAAAAAAAAAAAAAAAAAAGAGTTCTATTCCCTGGAGCTGGAGTTATAGGAGGCTGTATCCTGTCCA
GTATGGGTGTGAGAATCAAACTTAGATCTCTTAAGTGCTCTTAACTGCTAAGCCATCTCTCCGGCCCTAAATGAATGGA
TTTTTAAACTGTATACTTGCTTCCTAGGTCAAGCAACATTTTAATATAATCAAGTAACATTGATTTATTACATAGCCGT
TTCTCATTTTTTTTATGCATTTCAGAGTTTTGGGGAAATCAGAACATGTGTCTCCTCAAATACTTAGCATGTGTATCAT
TAACTAGAATCCAGTATTTGTTTACTTTTCTCTTAGAAGAGCTGTTGTACAGTGAAATTTGTAGATAACAGTTCCATCA
TGTGAGTCAGCCTCCTGATAGATGATGGCCTTCCAAAACCCTTTGGTATCAAGAGCATGACCTCATTTCAACAAGCATC
CTTGGGGCCCTTTTCAATGACCTCTACCCCAGCCCCACAGAGATAAGCTTGTTTTATTATTTGTCCCCAGAATATATTA
ATCTTTCCTTAGTTTGAGAGCTTTTTATACCTGGCCTTAGGGAACACTTAGCATACTGTCCATGTGTCTAGGTCATGGT
GCATGTTAGCTAGCTCATCCCTTATTATAGAACGTTCTGTCTGAACAGAGCCCCTGGGTGACTTCCCTTCAATGTGAAG
TAGTGTAACTGCTGGGCTAAGTATACATTTAGTTTATGTGGTCTGGATAGCTTTAAACTTGCAGGGCTTCCTACTTCTG
TCTCCTAGTACTAGAATTGCATGGTATTCACTATCAGACTCTTCCTAGAAGAGATTTGGTGTCACTTTATAGTCACTTG
ACTTTTAGCATCTGGTATGGCTACAGTGGAGGTAGTAGCATCATGCTTTAAAAGAATCAGTCTTTACCTCTCTTACTCT
GCCTCTCTTTGCCTCCCATCCTTGTCCTGCTAATTGCTGTTATTACCAGCATGTATCTCGTTGTTTATGAGCTTTTTTC
ACTTGGTACTCCAGATTGGCCTTAGGCCTTAAACTGGTTTTGTTTGTTTGTCTGTTTTCTTTCAATTCTGATGGAAAAT
AGGTTATAGACTATAGATCCTGGCATTTATCTGTTTGCATTACTTAACAAATAACAGAAGCAGCTGAACTGTCAGTTCA
CATTTTAGTCTTATTAAATAAAGGTTGTGTGTGTGTGTGTGTGTGTGTGTGTGTATGTATGGCTCAGAGGCCAAAGATT
TGGCCAAGCTCCACAAACTATTTCTGGCTTCCAGACCTTTGCTCTGGGTTCCCAACCTTTTTTTTCTGAGGAAATAGTT
GCTTTCCTACCTTGTTCTTCAAGCCATTTCTATTAGAATTTTAGCCAGCCTGTGGCACACATAAAAGAAACAAAATGAG
AAAGCCCACAAAAGCTAAATTGACCCAGGTCTGACCTGCCCTGTAACAGTTACTTGGTTTGTTAATTCTCCAGCGTCTT
TAATTAGAATTTAAGTTCAATTCCCAGAACCCAAAAAAGGCAAGCATGATGTTTGTAATCTTAACACTGGAAAGGCTCA
GTTCCAGGCCCATAAGTGACTAGTGGGATTGGCATGGTGACTCAGGCCTTTAATCCCAACATTTGGGAGTTAAAGGTAG
GAGGATCACTAAAAGTCTGAAGCATCCTGGGCTACAAAAGGCCAGCTTGTCTTATTTATTTATTTATTTATTTATTTAT
TTATTTATTTTATGAAAATAGAAAAATGGCTGCAAAGGTAGCCATTGCAAGGACTTGGTGTCAGGTAGAAACACTTAAA
TGTCCTTGCTACCTGATAGTTAAGCCCATTGCACATTTGTAAATGGGTGTAAAAACACTTATAGTAATTCACACTACTA
GCTACTGACCAGCCTTTCCCCAATTTAATCTATATTTTAATCCATGGAATCATTAAAATCTGAACTAGCCATGCTTTAC
CTGAACTTTGACCCACTGGCAAGCTTTGAGGGGAAAAAGAAACCTGGAGACATACATGCACTACAAATTTGAAGTTTTC
CTCTTCCCATTCCAGGAATCCCCTCCTTGTGCAGACAATTACCGTGTATCCGAAGCTGCTTTATTCCATAATAAGAAAG
GTGGGCCTGGATTACCACAGAAGAGGAAAACTCCTGCAGAGAATGTAGTCAAACGGCCTCGAGTACACTGAAGTGAGCC
ATACACAACCACCCAACGAATGTTTTTGAGTTTAGTGTGCTTGCCTAAAATGTATAAAGTGCACTAGAAAATTTTCAGC
TACAACCTGAACATTGCTGTGATGCATTTGTGTAGCAGTTTAAAGGCATACATCTGAAGGACTATGCTTCAGATTCTAA
TGATCCTGTAAACTGGATACCTTTAACCTGAATTTGAATCACTGGTTATTGACTGTTCCTAATGAGGCTGAAATAGTTT
TAACAAGGAGATTTGTTTCAAAAATGTCATAGTTCATAAAAGCAAAAGTTATACATTAGGGTTATTTCATGTTTTCCTG
ATTTTTATATATGTATTCTTGCCATTTAACCTCTTTAACAGCCTGAGCAGAGAGTTTAACAATGAAGCTGGGGCTCTAA
ACTTAAAATATGTATATATGTAAATGTGTGTATGTATAAACTTCTATCATTTGCCAAACTTGGGCACCAAAACTTTGAG
ATAAAGAATAAAAAAAAATAGCAACTTAAACTGTATTGATTTGTTCATTTAGATCTGTTGATATGCCAGACTTAAAAGC
TGTCTCAAAATACACTCACGCACCTCACCTGCAATCCCAGCACTCAGGTATTGAAGTAGGGTCATTGAGTCCAGCCTAG
CCTGGGTTGGAATGAGACGCTCCATTAAGCAAAGGACTAAAGAGGCATCAGTTGGAGACAGTGGTGGCATATGCTACAC
AGAGAAACCTTCTTGTCGGGGTGGGTTGGTGGAAAAAGCAACCCTTAGAAGGTGTCTCACAATTGCCTATAAAACTATT
AACTCCAGAAGCATCCGGGCTCACTCCATGGATGCCAGCACTCATGTGCAGACAAGTATCTTTTTAAAAGGTGACAGAA
ATTTGTTGTGCTTCAAATAACTTGTTGCACTAGCCCAGGTTACCCAAATAATGTCTTGGAATAAGGGAGATGGATAGCT
TGGCTCAGGATAAAGTAGGCATGAAGAACTGGTTAGGTCCTAAAACACACATAAATCCAGGTGTAGGGCCCCAGAATCT
AAACTGGGATTCACTGGTTAGCAGAACAGCTTCAGTGACTATCTCAAAAGGGCAAAGGCAGAGGGGCTCAGAGGACAGA
AGCATTGGCTGCTCAGAGCACTGGAGTCCCAGGAGGTCCAGTGCCTTGTGGTTCTATAGGTACCAAGCAGCACGCAAAT
ATTCAGGCAAAACACCCATGTTAGAAATAAATGTAGAGGGCCAGGGAAATGGCTCAGGCTCAGCAAGCCTGACAGCCAA
GTCTCATTCTCCAGAACCCACTATAGAAGTAGCTCAAGTTGTCTTCAGCATCCTCAAGTTTCAAAGACACCTGATCTGC
TGTTAGTCTCTGGCTTTCATATGCTCACATAAGGATCCACCACCACAAACTGTATGTGTATTTTTAAAAAACAAACAAA
AGCCAGGGATGTACTTTAGTTGCAGTGTAGGAAGCCTAGGTTCAGTCCCAGCACGGCATAAAAACCAGGCATGGAAGGA
GGTAATATAATAGGGTCAGAACGAGGTCATCAGCTACACCAGGTTGAGGCCAACCTGGGCCATGTGAGATCTTGTCTCA
AGACTACAATAAAAGATGAGACAAAGCACTTTACAAACCACAAAAGGTACTGATGTCTAGATATATAAAGAACTCAGTA
ATAGCCCAAAATGCTGGGAAGGATACACATAGTCACTTTAAAATGGTTTCAGGTCTGACATTATCATGTGACTCTGATT
TAGCTTTAGGTATACACCAAAAATAGGCAAAAGCATGTTGACATTAACACTTGGCAACTCATAGCAATACTGAGGCAGA
GTGAAAACGAATGTCCACTTAGGAACCCATACAACTGGTTTACTTAGCAACATGGTAGTACAACCATGGATGGCCTCAA
AAGCATGTTAGTGAAATGCCAATCACAAAATACCACCTACTGTTCATGTGAATGCTCAGAAGAGGAAAAACCATAGGCA
GGGGGACAGGGCAGTATGTGAGGCAGAACAACAAATGTCTGGGAATGAAGAGTTACAATGGGCATGTGCCCTTGAGAAA
ACCAAGAATTTTGTCAGCCGAGTTGTTTTATAACCCCAGGTAGCTTTGCAACAAGCTATGTTGATTAGGTTTGCCACAA
AATATCTTCCTGCCCATAAAGACGTGTTGAGATTTATAGATACAAGTTATTAAATATATACAAAATCAGAATCTCAGCC
AGCCTTAGCTACAAAGCAAGCTTAAGAGTAGCCTGACCTCATGTGAGACCCTCTCTAAAGAGCCTGTGGAAACTTAGAC
TGAGATCTTGAGTTCAAAGCCCATCTTAAATTAACTACACAGTAAGACACTTAAAGATAAAAAAGTTGTATGTAGCCTG
AGAGTAGAGTACTTTGCCTTGAACATTTCTGGCCCTAAGGAAGAGTAAGTCTTGTATTTTCTTTTAAATAAGCAGTTAA
GGAAAAAAACGACCCTCAGAACTTGATGCTCAGTTAAGAGCACTTGATGCTCTTGCTGAGAATCACCCATGTAAGGAAG
TTCATGCCTCAGACACTCTAGGGCCAACACATTTGTTTCTGGTCTCTATAAGCACCTGTACACAGTAGCATATGTTCAC
ATACATACATGAATATCTAAAGCCAAAGCACAAAGAGTAAACAGTAAATATTGATCCCATTACCAAAAATGGCTTAAAA
TATACCTTTACTAAGACTATAAACCAAATCCTAAAACTGGAAAGTGCTGTTCCTTTTAAAGGCGTGTAAAGGCTAGAGA
CAGCATTGGCCGCTCTGGACCTAGATCTGATTCCCAGTGTCCACACAGTGGCTAACCTTGTTAACTCCAGTTCCAGTAA
TTTCTTTGGCCTCCAGCACTTCTGACATGTACAAAGGCAAAACACTCATTCACATTTGAAAGGGGGTGGGGGGGGGGAA
CCAAAACCCTAAAAATTAATATTTTGTATAAGGAGCCCAGAGGTGGCCCAGAATACGTTTAACACTTTTTCATAAGGAG
TGTCTCTAAATTGCAGCCATTTTCACTCATGCCATTTAATATCTACAGAATGTGGCAAACATTACTAAATGCACTCTTG
GAGAGCAGGGTCATCTCCACAAATGGACTGCACACTGTTCTCCATTCCCTAGAGTTTAGGCTAAGGGGTTTCAACAGAA
AGCATCACATTCCACCATCCATTGGTTTACTGTTTATTCAATGTTAAATGTTACAGAGTTCTTACACTGTCAGAACCAT
GCCTGAGAACCCAAAGAAAGAAATGGTGCAATGGACAGCCTCATCCACCCAGACAGTGTATTAAAACTTGATTCATATG
CATTTTGATAATTTCACAATCTTAAAAAATAACAAGACTTGGAATTTACATCTGCTGCAGTTCTTCAGCGTTCCAGTAC
AGTATGCCATTAAACAAACCTCATCATGTTTCTGTTCACGTCTTAAACCATAACCCCAAACAGAATCTGTGTTTGTGTG
TCACGCTGGAGTGAAGCATACTTTAAGGGAAGTTTGTGGCTCAAGGTGTTAACGTTTCTGGTGCCCAAATGAAGTCTTC
AGTTCAACTAAGAATCTCAAGCCTCTAATCACTCAACCTCCTCCTGGCTGGTTCAGATCATGCCAACATTATTGCTCTG
AAGTGGCATACAGTCTGGTCAGGTGACACTGGCCTCCACCTCCTGCTTCCTACACTGCTGCCCTAAATCCACTAAACAT
GCTCTCAAGCTGGGAAAACATCCATTTCCTGTTCCACTCCTGTCAGTAGTGAGCAAGTCTGACCTGTGTTTGGCAAAGG
GATTCGTTTACAGAGAGAAACCCAGGCACTAGTATTGTATCACATAGTCATATGCCAACATAGAGTATGTGTACTGAAC
CATCTGTAGTGACAAGGTAGGTAACTGACAGGCTATTTCATTAGGTAGAAAGAAATTTAGCAGAAACCTCTTCTAAGAG
CCAAGTACTACTAGATAGTTAAATTGAAATTACAACAGAAATGCTACATTACTGACTATTCCCCTGCGTCTCCAAGTGA
TTACCAAACATACTCCCAAGGTGAATGTCAAGAGCTGGCTAAACAATGAATAGCCCATAACCTTCACATTTGGGGCACA
CACAAGGATTTATCAGACACCATGCCAGATTCATGCGCAACTTGGGTGTTTTGCTCCTGTTGCTTTGAACTGTTAAAAG
CCAGGTCAGTGAAACCCTCTCCTCCTGAGCAGACGTCACACTCTGTCCACAGCAGTGAGCAGACGCCACACTCTGTCCA
CAGCAGTTCAATTAGACCTGATTAGGAGGCTTGAGCAGCACCTGGAGTGCAGCGGATCCGCTGTCCTTTATATGCATCA
AAGGCTATCTGGTTCCACCTAAGGATTGGCAAATTCATAGGGCATTTTACAATTCCAATGCTTCTTTCAGACATATTTC
AGACAATATGCCCTTAAAGAACTTACTCTGAGTCAAAGCAAGGCCTTAATGTGGAACCGAACAAGCACTATCTGAATGA
AAAATTATTCAAAGAAATAAGACTACCCCCCCACCCACTCCCCATTTGGTGGACCAGAGTTTGGTTGAATTAAATCCAG
TTACCGCATAAAACTAAAAACTTAAATTATAAATCATAAGCCCCACACAGATGATCCAAACACATCATTTCTGACAGCG
GCTTGTTCCAGGGAGTCTAACACAGCCACAGCCTACAGAGTATGCCTTAGGATGGGGCACAGAACAGGTTAATTAAGGT
CACTTAACTCTTCATCTTTTACAGCGGCTCAGAACCCAGATGGCTGACTTCTGAAGGCTTTCTGACAGGAATCCAGTAG
AGTTGGTTTCAAGGTTCACACAGACACTAGTAGCTGTAACCTTATGTAGAAGATGTGATCCACATGAAATCCACAACTA
AGGGCAGCAAATCTGAGGTAGTCAACCTTGCGAAGGCTCAGTATCTGAACATAAAGATGTTTGAATGACCACTGGCTGC
AGGTTTGGTTTTATACGCAGAGCAACTATGGAAACACTGTTGAATCTAATTCACAAATATTTAAGACAATGAAACACCC
CTAAATCCAACACACATCAAGATCAGTCGTGACTCTTGAACTTCCATAGTGCAACAGGGACAGTCACTGGACTTGAGGT
CTTCAGTCAGTCACTGTAGATCCAGAAATCAAAACGATGAATGGCTGTTCTTGGTGGTTAGCTGTGGTCTAGTTACCTT
GGAAGGCTTAGCAAAGACCATTCCTTAGCTGTACTGTGTGTGGCTGTACTTCTGATTCTCAAGCACAGCATACTGGTTG
TCTAGCTGAAGTTTAAGGGCCTGGTTTTTTGAAACCTCCTCCCTATCTCTATTTTTGTGTCTTACTACTTCAAAGCTCT
TCTCCATTTCTTTATCCCTAAGGGAAAAGAAATGTAATCAGTAAATATTTCTTCTTCACTAGGGAATTTTGAAAGTACT
CTTCCTCGAATTTAAAAAGAAAAAAAAAAAAAAAAAAAAAAAAAAAAGACCCTAAGGATAATCCTGGAGAGACAGGAAG
AGAATACAAAAATCCTTATCAAACCATTTTAAGGCTTCAATTGAACAGAAAACATGTAATACTGTACTGAAGCTCCGAG
GTCCCATCTTCTGCATATTTTGGGACCAACTTGAAAACAACCAGAAAGAAGCTGTATAATGACATTCCTTCTAAGACTC
ACTTCTAAGTTTTCGCAGTTAGTAGATAATACGTCTTTATGCATAAGGGCAACAATTTCCTCATTCAAGTAATGTTCTC
ATCATTCAACAATTTCTCCTCCCTTTTGCTTTGGGGGAGTGGTCTGATTAGATCAGGTACAGAAAGATGTGTGTGGCAA
GTCACTGCTCTCCCAGAGTCCCCCACATGTGAGGACAGAACGGAGAAGCAAGTCCAAGTAGAGGTCTAAATTCAAGCCA
GCTGTGCTCTCAACGAGGTGAAAAGGCTGGGATTTACTTTAAAACCACCCAAAAAAATTTTCCATCTGATGCCATTTAC
ATTTCTTCCCACTTTATTTTTGAAAATTCATGTCCCTAATAAGATGTAGATGTGCCATATGTGGTCATTTAACTTCAGG
TAGTATTCATTTTAAGTACCTGCTGAATGAAATAAGATTATTTATAGTATTAGATTTATAAATGCCAACATCTTTCATC
TTCTCACATTTACTGTGAATGCAATGATAAATGCTTCTACAATTCTAGTTTCCATATTTAAAAGGAACAGAAGCCAGTA
ATAATGTACTTAAAGGACTCTTCCCCCACCTTCTCCTTCCCTCCTGGGTACCAAAGCCTCACCATGTGCCCAGCTGCCT
GTCAGAAAGCCTAGCTCATAGCCTCACATACTTACTTCCGGCTCTCTACATGCTTGGCAGTGGACTGCAGGGATGGGAA
CTGCGTGTCACTGTATATTTCTGGTGGTCCTTGTGGCGTTTTCCTTGTTGTGGTTAGCCTTGCCCCAGGAGGCCTGTAT
ACACCACTGGGCATTGCTGGCTCCGGGGTTTCTGTAACTAATAGATAAACCATTCAAATGTTTGACTTTCTTGTAACAT
TTTCCCACACTCCTGATCCTATAACAACTGTAGGAAAGGCCACCCTGATTTAGAATGGATTTGGACTACACAATAAAAA
CAAAGTTCTGTGAGAAAATGTCTCAAGTAGAACCTTGCCCATCTCTTAATCTCTTCTGAAATTCAACACTGCTGTCGCT
GGTGTTTCTATCAAAAGGTGACATCTACTTTCTCTACTTGAAAGGTGGGCTAAACAAGGTATTTTATATAAATTATTTA
ATAAAATACACATGCAGTATTTTAAATTCCTAAAAATAATAAAGGTCTTTATTTCATTAAGACTATTGGATGTTAAGTT
TCTCTATTTGCTTATAACACAGGAAAAAGCACCTTGACTTTAAATTTACTGTGACTGAATTTGGGGGCCACAATGTTTC
AGGTTTTTCCTGTCTCCTACCTTCCCAACTGACCCTTGATATGTATTCAAGCAGTGTCGTTTCCCACTTTATGAAAAAT
TTCAAAGTTTCAGGCTGGGGAGGCTCACTGGGACAGCACTGTAATCATCTACTAGCTGAAATAAAATGTTCTTATAGTT
TAAAGGATTGCTTTCTATAGGCTTTGTTATGCCAGAGGGAAAGCATACAAGCTTTTACTGAGTATCTAAGCTGCACTTG
AGATACACACATTAAAAACTGCCTTGAGGCTGGCGTGGTGGCGCACACCTTTAATCCCAGCACTTGGGAGGCAGAGGCA
GGCGGATTTCTGAGTTCAAGCCCAGCCTGGTCTACAAAGTGAGTTCCAGGACAGCCAGGGCTACACAGAGAAACCCTGT
CTCAAAAAAACAAACAAAAAACAACCTGCCTTGAACAGTGGAACATGCTTGCAACATGGGAGGCTGAGACAGGAGGATC
TGGAGTTCAAAGCCAGCCTGGGCAACTCAGACCCATCTTCAAAAAAACCAGACCAAACACTAAATAACTACTGACCCTT
ACATTTCAGCCACCACATGTGGCTTTTTAAAAAAAATTCGTTATTTGTGTTTGTGGGTGATATGACTTGTGAGGCTTGC
CTGCTCTCTTATGCACTCATTCATTCATATGTTTTTTTAAAGATTTATTTATTTATTATATGTAAGTACACTGTAGCTG
TCTTCAGGCACTCCAGAAGAGGGTGTCAGATCTCATTATGGATGGTTGTGAGCCACCATGTGGTTTCTGGGATTTGAAC
TCGAGACCTTGGAAGAGCAGTTAGTGCTCTTAACCGCTGAGCCATCTCATCTCAGCCCCGTCTCTGTAGCCCTGGCTGC
CCCAACTCAAACACCATCTGTCTGTGTCCTGAGTGCTGGGATCAAAGCAACACATGTAGCTATTTTAGATGAGTAAAAT
GAAATCTAGGTTTCAATTCCTCAGTCACATTAGCCACCATGTCCTAAGTATTCAACACAGCTACATGTGACTAGCCATC
CCTCTCTCACTTGAGCTCATCCAGCACCCCCCAAAATGCTACCTAATGGGCAGTATATTCTAGAAGAGACAAAAAAAGG
GGAGAAGGGGAGAAGGGGTGCCATGCCAAAAATAACAGCAGGAGTACAAAGGAGCTACACTTCAATGTGTACACACATC
ATGTAGATACACAGATGCCCCTGGTGGCCAGAAGGGAAAGGTGGATTCCTTGAAACTGGAGTCATAGATATTTGTGGTC
CTCTGATAAGAATTACTAAGATATAATTCTGGTGCTGGTAGAGTGTAGGTTAGTACTCTTAACTGCTGAGCCCCAAAGA
GGTTACATATTAAAAGGGGGCTTGGGAAGGGTTTTATTAACAGGGTAAAATTAAGCAGATATGAAAAGACCCTCTTTAT
ACAAACCTGGTGGCAAGATCCAGCTCCACAAACTCACCACACAAATAATAAAAAGAAAGCCAACACTTAAAACCAAATA
TTAGTGAACAAGCTGAATCCGGCCCAACAACTGAGCACAGCTATCCTCACTGCTGTTAATTCCACATTACTGTCTATGC
CACAGCAGCACAACAGAGACCACAGTCATCTACCACCTGGCTTTTCAGGCACCTGATAAAAAGAGGAAAGAGAAAGAAA
ATACTCCCAAAGCTAAGAACTAAGGGTCTGCTGGAGCTGTGTGTCTGGTGCATTCCAGGAGTAGCTTGAAAGAACATCA
GGTTCAGGAGAAAAAAAAAAAAAAAAAAAAAGGCAGCATTTCCATGGTGATTCACACTAGTCACTTCAGCACTTGCCAT
AAGCATTAGAACTTAAACTCATGTGCACCAAAGCCCAGGCATGGCAGCAGCACATGCCTGTAAACAGCACAGGATGGGT
GGGTGGTGACAGAGAAAAGAACATCCTAGGAGTGCGCTAGCTAGCTAGTACAGCCATTCAATCATCTCTAGACTCGGTG
AGAACTTGTCTTAAGCATTTAGAGTGACGAGGAAGACACAGCCAACCTCTAGCCTCGATACCCACACAACAAGACATAA
ACACACACACACACACACACACACACACACACACACACACACACACACACACACACACACAAATTCAGGGCTAGCCTGA
GCAGAGTGGGGATGAATATTACCAAAAATATACTGATGGCATTCTCAAAAAATAAGAACTTTACATATAAAAAATTTTT
TTAACTTAGAAAATTGCCAGGAAAGCTCACACCTGTAATCTCAGGTTTTGGGAAGTGGGGTAGCATAAGGTCATCTGTA
TTACAGAGGACTCAAGGTCAGCCTGAGCTATATAAAATAAATAAAACTAACAATTTTGAGATGAAAATGCAGCTCTGTG
GTACAGCACATTTAGCATGTGCAGAGTTTTTAGAGTTCAATTTCTAGCACAGCAAAAACCAGACAAACACGCTGAGCTA
GGAAGCTGGCTCAATGGCTACAGTCTGCCGAGCATGTGTACAGGATGGTCAGATCCATGTAAGCCTAGGCAGCCATGGC
AAGGCACACAAACAGGATCCTAAAAGCAAACTGGCTAGCTAGGATAGCTAGAACTGGTTTGAGTTCCCGGTTCCTTGAG
AAACCCTACTCTCAAAAAATAAAAATGGACAATGAGACCGGCACCAAGCTGTGGTCCATCCACAAGCACTGTACAAACA
TGCAAACACACATCCAACAACACACATAAACCCCTAAAAGTTCTTTAGTTCTAAAGAAATTTTACTCACTCCCTTCTGC
TGAGAATGTAAGCTTCAAGACCTAAGTGCAGCACCAAGAATGTGATTTAGTGGTAGGATGATAGCAGGAGGTGAACTAC
TTGGCTTTATTCCCTAGCACCAAAAACAAAAGCAAAACTTTGACAGCCTACAGGACAAAAAAAGTATGTCCCTCAATTT
CTACATTCCTTCCACTAAAGCAGCATCCATTTGGATCCACTATGCCCATCCCTCAGGAGGGAAAACAGAAGATCACCTG
TTACTGGAGCAGGAGGCGCTTGTACCGGAGCGGTTTTATTCCAGGGACCTGAAGATTTTTCTGCTCCACTGCCACCTCC
ACCTTCTTCCCAATTATCTCCTGGATCTTCTCTCTTCTCATTATCATCATCTTCCTTTTCACTATTGAAAAACAAGCAC
ATTGAACTTCCCAAGAACTGGAACTAGATGTATGCACCACCATATGAAACTTCTACAAAAAAATACATTTCTTTTGAAA
ATATGGTCCAATTATACAGTCCTGACTGGCATGGAATCAGATACGTAGGCAAAGCTGATCGACATCTCAGAGACCTGCC
TGTCTCTGCCTCCCTCTGCTGAGACTAAACTCTTTAGGGGCGAACTTTTGGGAATCAGTTCTCTACCATGTGGATTCTA
GGGAATAAACCCAGGTCATCAACTTTTAACAGCAATACCCAATAATAAGCCATCTTGCTGGCCCCCATAAAAGATATTC
CTATTAAAGCAAGATGTGTCCTAGCTTTAAGCCAAATTTCTTCTGGCTTAGTGTTGATTTTTTTTTTCCCTCCATTGGT
GAGTGGAGTCTAACTTGGATTCTCATGTATGCAACAAAAGCATGCTAAACTGAGCCATACCTAGAATCCAAGACAAGGC
CTCACTCAGGTAGCCCAGGCTGGCCTTAAACTCACTATATACTCAAGTAAGCTGACGATCCTGACTGATCTTCCTGTCT
CTATCTCCCAAGTGCTGAAATTAAAGGTGTAAACCACCATACCCATCTTTATTAAGGCCCTTTAAAGACACTGCCCACC
AGCTGGAAGTAACCCCAGCATTTAAAGACTTATGATGTCAAGAAAGAGGAACAACCTAATTTCCAATCTGTATTTAAGA
CTAAGACTGTCTCTCAGGGCTCAATAACAGAGCACTTCACCTGTAACTCAAAAAGACAAACAGAGAAGTAACCACCGTT
TGGAGGCTGACTGGTGACCTCAGCATGCAAGCTGAGGCAAGAGAATGATGACGTCACTGATACTTTCTCCAAAAACTAA
AGGTCACACCTAGATGGTCCTTGCCACCAAGCCTAACTACCAGAGTCTAATCCCCAGGATCCACAAGGTGGAAGAGAAC
TAACTCCTGTTGGTTTTATTCTGATATGCATGCCACAGCATGCTCACACAAAATAAACTGCAATTTTTAAAAGACAAAG
GAAAAAGAGAAAGCCAAATTTCAGTTTTCCCTTTTAACATACAACCTGTCCAGCCATATAAAATGATGCTGGTCACTAA
CAAATAATATTCATGCCATTTAAATCATCATCTAATACATTATACTTATCATAAACATCAAACATCCAGAGAGAATCCT
AAAGATCTGTAATTTTAATGAGAACTTTGATAGCTAAAACAATGTTAGGAACTGATTGCTCAAGTCCAGGTTCTGCCAT
CTCTGTCATTTAATGGTCAGGCTTTCAAGATGATACTGGCTGGCTTTTAGATATCACCAATACGAAGAGCAAATTTATA
CACTCAGTACATATATCACCTTCGTAACAGGAAGTAAAAAATTCTAGCTTTCATTTGAAAAAGAGATGGGAACATGTTA
TTCTCTAATGGATCGTAAAAATCTGATCAGATAGCACAACACCATTGTCTAACAGTAGCTTATTAGCTAGTATGAAGTT
TATAATTACCCAGGCTAATTGTGGTTTCCAATCTTCATAATTTTTTCAAGAGGTTTTTTTTTTTTTGCCTTTTTAAATT
GTAAAGCCATGTCAATACTATAATTGCTTCAGGGATGAAATAGGTAATCATCTGGGTGTGGTAATATCCCAACAACCTA
ATGCATCCAAAAAAGGTGAAAAAGATTCCCACCTACATGGCATTTACACCTGCATACACTGAAGCAGTTGCTTTCAGTT
ATTCATTCACTCTTTGAATCTATTCACTCACCAACACAGTAAATCACAACTGTTCATCTGTATATATGGTAAGAACACT
TGCTGTAAAAGTAAAAAGAGCAGAGTTCAAACCCCTGGCCCCCACATGAAAGCCAGGCATGTCAACACTGCAGGGCAGA
GACAAGTAGATCCTAACAGTTTATTAACAACTTCCTGACTCTAAAAAAGTTAAAATAAAGAATTTTATATCCTGTTAGA
AAACCAGTTCAATTTAAATGTTCTCCATAATTTAACAAAATCAAATTTTATACTGAAAAAACTTGAAGGCCACCCCTCT
ACCTTCAGAAAGTGTTACTTTTACCCACTAAAATTCAAGTCAATATGTCCAGAAATTACCACTCAAGGGCATAATTTAA
CTTCAGAGCAGGGACCACATCCAGATAAAAGAAAGGAACCTGTAAACTCCCTCTCCAGATTTATAGATCAGACTTGGAG
ATACAAAACAAGAACATTTTCTACTTTTTCTTTTAAGTTTTGAGATAGGGTCTCACTAAATTGCTCAGAGTAACCTTAA
ACTCACTCTAACCCAAGCGCACCTGATTTTTCTTATTCTCCCCACCTCAGCTTCTCAACAGCTGACAGTTAAGGCTTTC
ACCACCAAGTATTTTCTAAAATTACTCATAAGCTTATATACTGCTCTTGTAGAGGATACAAATTCAGTCCCTAGAACAC
AGCTCCCAACTACCTGTAACTCCAGTTTCAAGGGAACTGATGCGCTCTCTTCTGGCCTCGACAGGCACCTGCATGCACA
TACATGTACATATACATCCCAGACATACATATGTAATTAAATCTTTAAAAAAGGAAAAACAAAGCAAAACAAAACACTA
AAGCCACACCAAAAAGGGAGATAACTTCTAAGTTCCCTACTATAAAAAACAATGTTTAATTTGGTAAATTCACAAGTGA
GAACACCTAGGGATATGAATGCCTGGGTGGTACAGACACTAACCATACATTAAAATATACTACCTGGCTTTTCAATAAG
TTACCTCAGATCATATATAATCTAGATTTTAAAAGATTAATTTAGAGTTAAGAAAGTTATTTCCTCAACAACTGTCCCA
TATGGTCACTGGAATTCTCTACAAATTGCAAGCTTCAAACCAAAGACAATTTCCAATCATTTTGCTTGTGTAAGATGAG
TAAAGGATTTTAAGGCAATAGAGCCCTACTCACTTTTGAACTTCAAAAACCTTACCTTAAATACTTACCCTTTCTACTT
AAAAAAAAAATCTTTCCAAACATGTTACTTTTAAAACCAGATCTAAAATGAACTACCACAGATTGAACGTGACAATGTA
GGTTCTCTTAAATCAACCAATCCAACTGTGAAAATGACTGTTTTCCTGAACTTTGAAAACCAGTCACCTCAGAGCCCGA
AATGGCAGGTAGCACAGGCCTGCAATTCCAGCAGCTCAAGTTCAGTCCGGAATACACAGCTAAGAACTGTGCAAACGAG
GGCTAGGGTAAGCTACTTTTGGGGATAGGTTCAGAAGAGCCTCTATTAACGTTCAATTTTTTGACTGGGGTAGTGGGTA
TTCAAGGTGGCTTCATGGTCAATGCAAATTCGTGTATGTTGGAAGTATCCTACACTGCAATTACCAGAGAGAACTAGAA
AACAATTTTACTAAGTAAGTAAAAGAAAAAACTAAGTTCCTGCACAGCTTGGTGGTACATTTGGGTGGACACTTTAGAA
TTAAGAGTTCAAGGTGACATGTCGCTCAGCGGTCTTCCACAGATCAGGGTCCAGCTCACAGAACCCACACTAACAGCTG
TAATTCCAATTCCAGAAGACAGTACAGACATGTATTACACAGGCAACTCGCTTCAAATATTTTTTTTTGGAAAAAAAAA
AAAAAAACAAGCCCCTTCTTCTCCTGAGTCACACAGTAAGTTCTCAGGGCAGCGAGTGAACTACAGGAGCCCTACTAGG
GGTAGGGAAGACCACCTAAATTCAAACACGAAGTGTTAAAAATTTTTAAATGACAAAAATCCATTTTAGTCAAGCCAAA
AATCCACATAGCTGCAAATAGGGAAACCACAGGATTTTAGAGTTGGCAACTAATTTAATGACACAGAGGGCATTTAAAA
AAAAAAAAAAGTAAACGCGTGCACTACTTAACAAGGTTACCTAATCCCACGTGTAATCACATCCTACCTTATTTGCATT
GCCTGAACTCTTAATCCGCTGTAATCAACCTCTCTTTGCTCAAATTCTTTCCACTCATTTTCATCCTATGGAAGAAACA
ACACCTGATTAGTTTTTGGTAAGCCCACTTCAGACTAAAAGGCATCAAGGGCCAACGGGATTCCCCAAACTGGTAAAGG
TACCCGCGAAACATCGAATTAATTAATTAGAGATCTTGGTAAACAGATTCCAGACTCGGAAAAATTCCATCAGTGGTCG
CCAGGATTCTTGAGTCGGTCTAAGAATCGGCGGCAGTCAACACCGAGAACCAGAGAGTCTAACTCTCCAGGCCCACGTG
GTGGGAAGAGAGAATTTACCCCGGCCTCTAACCTCTCCGCACACGTACACAGGAGTGCGCGCACACAAATAAGCAAATA
AATGTGATCTTTAAAAAAAACAAAAAACAACAACAACAACAACAACAAAAAAAAAACAAAATTGCAGACAGTGACACAG
ACCTTTAATCCCAGTACTCCAGAGACAGAGGCCGGAGGCTCTTTGTGAACTGGAGGCCAACCAGAGCTACACCGTGAGA
CCTTGTCTCAAAAATTGGGGCTAGTGGCCGGACGGTAAAGTTTAGCCCGTTCCTGCAAGATCCGGAGACGTGCCTAATT
CCCAAACGCCCTCTCTGCCGCCGCTTTTACTTAAACCCAAGCCCAGGGCGAGCTGAGCGGCGGAGAGAAGGCGACAGAA
ATTCCACAACTCAATTCCCAGCCCTGCGTGGCCGTGGTTGGGAGAGTAAGCCACGCTGGCCCGGGCGGCACAGAGGAGC
CTCGGAACACCTTAAGAACATGAAATCCGGCCCCAGCGGGGCTCAAGCGGGACCACGAGGACTCGGGCACACGTCACCC
GGGGACCGGCCCAACCCCCGTGGCCCAGAGGGCGCGGCGCGCAGAGGGCCGCGGACGGGGGCGGCCAGACGAGCCCCGG
GGGCCGAGGACCGCGAGCCCGCCCGCCCGCCGGGCCGACGTGCGAAGCCCGAGTCACGTGGCCTCCCGGGCCGGCGGGG
GCGGGCACATGCGGCCGGCCGCCGGCCTCCCGACCCTCACCTTCGTTATGGCCTTGCCGGCGGGGCCCGCGCTCCCGCC
GTCTCCGGACCTCGAGCTCCCGCCGTCTCCGGACCTAGCACCAGAGCCCGAGCTCCCGCCGTCGCCCGACCTGGAGCCG
GAGCCCAAGCTCCCGCCGTCGCCCGACCTGGCACCGGAGCCCAGGCTCCCGCCATCGCCCGGCCTGGAGCCCGCCGCCG
CGCTGCTCCCGCCCGCGCCGCTCGCCGCGTTGGCCGCGCGACTGCTCCGTTCCTTCTTCTTCTTCTTGTCCCTCTTGGC
GAAGAAGTTGTCCAGGCTCCGCTCCTCGGTCTCGGCCATGGCCGCGGTCCGGATGGGTGGGGCCGGCGGAGGGCGCGGC
GCTCGCGGCTCCGAGCTCTGGGTCAAGGCCGGCTGTGCGCTCAGGCTGCAGGGTGCGGCGTCGCCTCAGCCCCGCGGGG
TCGCCGAGGCGCGGGCGGCAGGGCGGCCGGCTCCCCCCGTGCTCTGGTGGTACGTTCCTCGCTGGGAACCGCGAGCGTG
GAGCAGGGTCGCGCCCGCCCCGCTGCCTGAGGGACAAGGAAGGCGCGCACCGGCGAGCGCTCGGCTAACGACGGCGCGT
GCGGGAGGAAGACGCGCGGGCCTGAGGAGGGGGAGAGGGGACGGGGAGGGGAGGGAGGGGAGGAGGGAGGGCGGGCCAC
CAGCAGAGAGAGGCAGCGCCGCGCCTGCGCGCTCGGTTCCGCCCGCCGCTGTGGGCGCGCCGAGGGGGGCGGGCGGCGG
GATGAGAAGGGGGCGGGTCTTCCGGGAGAGCCAATCAGATGCCCGGACGGAGCGCTTAGGTGACAATCCGTTCCTTCTA
CGGGGCGGTGCCCTGTATGGGGGCGGGGAGAAAGCGGGTGTAGGCAAAGAGAGTGACGTCCCTAGTGACTCCTGCCTCC
GATTGGCCAGGGAATTTGCCTTGAGGGCGGGCATTTAGAGGGTTGGTGACTTGGCTGAAGAAGACCAGCCGGAGGGGGC
GGGTGTGCCTTGGTTGCGCGCATGCGCAGGGTACACGTGCGTCTGCTCGCCAGGACTGTGGCCTGGTAGGCGTTTGAAC
TTTCAGGAAAAGGAAAGAGACCATGTTAAGCCAGCTATGTTCAAGCTTGGTCCTCCTGCCCTGCTTTTCTGTACTCGCA
CCCTCTCTGGTCGGGCACCTATTACTTTTCTTCTGAATGGAATGCTTTTGATGAGGAGCGCTGGATTAGTATTCGTGGT
TCCATTTACGGAAGGATCTTAAATAAGCCAGTGCATGAAAGGAAGGGAACTGATTTTGACTAGCTGTGTCTACCAGGGT
CAGGCTAAATAAATCTGACCCTCTAACGCTAGCTCTTGTGAGATGGAGAATGGCTGGGAATGTGATTTTGGTGGTAGCG
TGCGACAGGCGCATGTCAGCGCAGAGTCTGTCCACAGAATCACAAAAAGAAAACACACAAAACCCCTCCTTTTCCTGGG
TCTTCACCCTATTGGCACATACTATGTGAATACTTGATCCCCGAAACATCTGCAGCCCAGAAAGCTTTTTGTTTGCTTG
GGGGAGATTGCTTTGTTTTGGGACCGGGTCTCACCTACCCCAGGCTGCTTTGAACGCCATACATAGAAAAGATAATGTG
TTTCTGACCTTCCAGCCTCCACCATCCCAAGTGCTGAGAATTCAGGCATGCACTTTTGTGCCTATGTTAGCGATACTAG
GGGTTTCCATTCTTGCTAGAGAGGCTCTCTACCAGCTAAAGAATGAATGACCCAATACAGATGTTGGAGTTTCCTTGTT
TTTAGTTACACAATTGCACCTAGAATTGCTCCTGCTCTGACTTGAGTTTGAGTGATTGCTACAGTTTAACTTTGGCTCC
TATGCTGGATCTTAGGACAGAGAAGGGGATAAAAAATATCCTTGTTTCCACCTGACTTGACTTTGGGGCCTTGGAGAAT
CAAATGTGTGTTTCTCTATGAGACGTGTGGTCTGGGTATGCATTAAACTTCTGCTTCCTCTAAAGGGTAGAGGAGAAAG
CTGTGACAACCACTGAGGAAATGGCTAAGTCCTATGTCTGTAGCCTTGGGGGAAAGATAGGACCCAGCTAGTAAATGGA
GGGATGTCTTTCCACACCTGTTCCCTCCTGTCTTTCCTTACAAGTCCTTCCCTAGTTGTGGCTATTGAAGACCTACATT
CTGAACATATCGATGGATAAATGAAGGCTCTATGGCTGTAGCCAACAATAAGAAGACCTTTCTTTTGTTTTGTTTTTTG
AGACAGGTTTCACTATGTAGTCCTGGCTGCCTTGGAACTGGCTCTGAAGACGGAGATGACCTCAAACTCAGAGATTCAC
CTGCCTGGGCCTCCTAAGTGCTGAAATTGAAGGTGTGCGCCACCACACCTGGCTAATAAGCAGACATATTTGTTCTCTT
CCCCCAGCCCAGGATGGTTCCTAGGTGACTTCCTTTTGTGACACCACATATCTTCCTGTGGTATTGTCCAAGTTCAGCA
CTGCCCAGTGCCACCAGCTTTGCTTAACATCTCTTAGCAAACTTTCTTTTTACCTTCCTTGTCTTCTTTCCTCTTTCCT
TCTTTTTCTGCCCTTTCCTGCTCCCCACCCCCCTCATTTTAAGACAGTGTCTAAAGTATCCTAAATTAACCTTGAACAC
AGAGCCAAGGATGGTCTTGAATCTCCACTTCCAATTCTCTCTCTCTTTCTCCTTTCTCTCTCATTTTGTTTCTGTTTCC
TTTCATTTGTTTTTAGGATAGAGTCACCCTGTGTAGCCCTGTCTGGCCTTGAACTCAGAGATCTTCCTGCCTTTGCCTC
CCTCTGCTGAGGTTAAATCCCACCACTGTCTACAAAGCTGTTTCCTGTATCCTGCAACTATTTTATCAGTTGAACACCC
CACCCATTTTTCTCCTTTCCAAAAAGATGTATTTATCTTTATTTTATGTGCATGCATCAGTACATGGATGTGACTGTAC
ATCTGAACCCCTAAGCCTGGATTTACTGTGAGCCACTTGATATGGGAGCGGGGAGCTGAACACAAGCCTCTGGCAGAGC
GGTAAGTACTTCTCATCACCGAGTCCTCTCTCCAGCTGGCTCTCTCTCTTCCTTAAACAAGGTCTTCCTGTGTAGCCCA
GGAGGGCCGAGTGATAGATAACACTCCTGTCTCTGTCTCCAAAAGTGTTGAGGTTCTAGGCCTACACTGCCAAACGTGG
TTTGCTGTTGTTTCTCATTGCTGGTGGTCTAGGTGAATGCAGGTGTGGTACACAAGCACCTGTGTGTTCATGATCGCCT
TGGAGGCCAGTGGACAACCTTAGGTGTCCAGGCCTCAAGTGCCGTCTACATTTTCTGTTTGTTTGTGTATATATGCATG
TGATCCCAAATGACATGGTGTATATGTCGAGGTCAGAGGGCAATTTGCAGGCAGTAAGTACCTGTATCCACACAGCCAT
TTTGCCACTCCACCTTTTTTTTTTTTTTTTTTTTTTTTTTTTGATGGACATAGTTTCTCACTGGGGTGCACTTGGAGTG
CACTAATTCAGCTAGGCTGGCTGGTCAGTGATCACTAGGGATCCCCCTGTCTCTGCCTCCCCAGAACTAGGGTTACTAG
GACCCCTGTAACCTGCCAGGAGGAGGGTAGCCAGGGAGGCTTAAAAAGAGCAGCTCGAGAGAATAGCTGCCATTGGTTT
CTGGGCATTAGAGTCAAAGAGCAAGGGACCCGTTTCATGCTGCAGAGACTAACTACAGGTGACCCAGCCGCAAAGACAC
TACTCTCTGTCGATCCGACCTCAATTTCTCCCCCACAATGGCGTTTGGGACGTATGCCATCCATCACACTGCAGACCAC
AGGAGGGACTGCTGTGGCGTCAACCCTTGGCTGCTGCTCCTTCTCTGCATTTGCTTGCGCCATTGATCCCTTCAAGCCG
GTCATGGATTATTGATGCAGGTCCATTTATTTGTATCTAGATCTTGTCTTCCAGATTATTAAAGAGTTGAAGGCAGGCA
TTACTCAACTCTGAATAAGGCACAGTGTCTCAAGTAAGTGCTTCTTCATAAAGGAAGAGAGCCGGGGAGGGAGATGGCT
CACTTAATACGCAGCTTGCCTTGAAAGCGTGAAGGGCTAGGTTCAGTCTGCAGAACCCACATAAAAACTGTATCCCGTG
CAGTACCCCAAATCCTGCCATGGCCACCCCTTCATCCTTATCCTCCAAAGCGTTTTTAGCTCTTGCAGGACTCTTCACA
TCAATGTTCCAGTGTGTGCACACTGTCTCCATTCTTAAAGCCAAGCAAATCTTCTCTTGTCCTTGATGCTGGGAGAGAG
GAAGTGACCAATTGAGGCCATTCTAGGATATTTCAGGAAGTCTGGGAGAGGTTTTAGTTGTCTCTTTTGAAACAGGGTC
TCTGGTATGTGTATAGAGTGAGGACATGATCACTCCAATGTACAGTTGAAGAGAAAGTCTTTATTGTAGACATGAGGGA
GAGTACAGCAAAAGGCATCTGGAAGAGTCCAGAGCAGAGGAAGTAGACTGAACATGACCATGAGAGAAGGCGTGTGCTG
TGTGTGTGTGTGTCTCTGTGTGTCACACACACAAACACACACACACACACACACACACACACACACACACACAGAAGAG
ACAGGATACCAGTAGCATGGACTCTGTATCAGATATTTGTAATGCTGAGAGAACCAGAAGGCCAACATGGGCTTTGATA
TGCTAATAGGTACCACAGTTAGCCATTTGTCTTGAGTTTCTTTTGAACCTGACAATACATAACCCTGGCTGGCCCAGGA
TTTGCTCTATAAACCAGGCTAGCCATGACCCTGCCTCCTGAGTGCCTGGACTTAAAGGCATGTACCACCACAACAGGCT
TCTTCAAAGACTAAGACTGCCCAAACATGGCACTTCTCTCTGGGTCCTCAGAATGCCCAATTTGATGGAGGTCAGTTCT
CCCCATTCAGCAAGGGCTGCTGAGCTCTGATATTCTATCGAGAGCTAGCAGAAGGCAGCTGGGCAACCCCATTGTTGCT
GGGTTCTTTTCTGCTGAGCTATTTGTTCCAAGTTCCATTCTGCTCCCGAGGCTGGAAATTCCACCAGTGCTCAGCTCCC
AAGAATGCAGCACACACTGGCCAGACAGCTCAGCAGGAAGCGGGTGACCAGGATCTGCCGCTGCCTGAGTGTTCTCCCC
CAAACTGGCCTTACAGGTCTCACCTTGGGGGTGGTGGACATGGGTGCTGTGTCTCCTATGGTCACCACAGGCAGACAAG
GTATCTGACCTTACAAGGTCTTGGACCCTTTCTTTGGGTGATTCGATGCCTTGCTGATAGTCCCCTGATTTCTATTTTC
TTCAACCACTCTCATTCAGTGATCTAAAACATCTTAGCAATGTTTACTATACTACCAGGCCCTATGTTAGGAGTTAATG
ATAGGAAAAGTATAGGTTGGTGGTCGCTGCCTGTGACCTGGACTGCTTGCCGCTGGACCCTACCCACCCTCGGAGGTTG
GAGGTGAGGTGCTGCAGAATCGATGGCTCAGACTGGGGGAGCAGGTGAGAAACACCACAGCGAGCTGTTATGAACGCTG
CTGCAAGCTCCTTTAAAATCCTCCACCAGCACACCAAGAAAGCATCTCTTCTAAACAGCAGTTCCTGGTTGGCTGACAT
TTTCTGCGTCACCAATCCTTGGTCTCTCAGGCAGTCCTCAATTAGGTCCTCCTGCAGGCGATGCCTTTGCAGCTGTGCG
GCCTCCCTAGCATTCCTGCAGAGGCAGCCCCGCCATTCCTCCTACACGTGGTAAAGCCCTTGCCTACCCTAACGTGTGG
GAGGTACTGGGTTTGACCTGGAACTTGAATTCCTGATCCTTCTATGTGTGGTGAATTTTTTTTTTTTTTTTTTTTTTTT
TTTTGTCAACATAACACAAGCTGGGGTCATCTGGGAGGAAAGGACCTCAATTGACAAGGTGTCTCTATCACTGTGGCCT
GTAGGCATGTCTGTGGGCATTTGCTAGATTAGTGGAGGACCCAGACCTTTATAGGCAATGCCACCCTGGGCAGGAGATC
TTAAGTTGTATTTAAAAACAAAAACAAAAAACAACCCAAGCTTAATGTGCCCATGGAGTAAGCCAATAAGCAGCTTCCT
CCGTAGGTCTGCTGCTTCCTGCTTCTGCCCCTGCTTGAGTTCCTGCCCTGCCTTCCCTCAGTGATGGATTATGACTGGG
ACATAAACCCTTTCTTCCTCGAGTCGCTTTTGCTCATAGTGTTTATCACAGCAATTAACAGCAAAGTAGGCGACCATGC
CTCCGCCTTCCTCGGGCTGGAATTCCACTTGGGCGCCACCACACCTGGTTTCCTTTTGCTTGCTTGGTTTGGTTTGATT
TGGTTTTGCTCTCTCCAGGCTGGCCTCAAAGTCCTGGGCTTGCATGATCTTACTAGCTTACTATCCTAAATAGCTAGGT
CTATCTTTGACTTGATACAACTTCTTTTTTTAATTATGTGTGATTGTGCTGACTAGTGTTGTGTCAACTTGACACAGGC
TAGGCTCCTCAGAAAGGAGGGAGCCTCCATTGAGAAAATGCCTCCAATAACATTGAGCGGTAGGCAAGCCTGGAGGGCA
GGGCCCAGTCTACTGTAGGTGGTGCTGCCTTGGGCTGGCTGTCCTGGGTTCTATAAGAAAGCAAGCTGAGAAGGCCAGA
AAGCAGTAGTCTTTCATGGCCTCTGCATCAGCTCCTGCCTCCTACCAGGTTCCTTCCCTGTTTGAGTTCCTGTCCTGAC
TTCCTTTGGTGATCGACAGTACTGTGGAAGTGTAAGCCAAATAAACCCTTCCTCACAAAGTTGCTTTGGTCCTGGTGTT
TCATCACAGCAACAGTAACCTGAACTATGACTCTCTCTCTCTCTCTCTCTGTGTGTGTGTGTGTGTGTGTGTGTGTGTG
TGTGTGTGTGTGTGTGTGTGTATCAGTACCTGGGAAGCTATAGAGGAAGGTGCTGGATCTTCGGGAGCTAGAGTTAAAG
GCAGTTGTGAGTCACCTTGTGGGTGCTGGGAACCAAACTGGTCGTCTGCAAGAAGAACAAGCACTTAACCACTGAGCCA
TCTCTACAGCCCAGTTTCCATGATTTTTGGTTTTTTGGAGGTATGGTCTCGCTATGTAGGCCAGGCTGGCCTTGAACTT
GTGAGCCTCCTGCTATTGCTTCCTGATGAATGGGATTTTTAGAGTTGTGCTGCTGTGCCCAAGTAGCTCAATCCATTTA
TTGTATAAAGGACACAAGTTGAAGGGTGAGACGATATTGGAGACTAGAAAAGGAAAGGAAAAGATAGGATAAAGGCTAC
AGGACCTGAAGCATCTCCCACCCCTGTCCAGAAAACAGCAGTGTGAATTGGTGCTCTCTTGGACCAGCCTGCTGTGAGG
AAGAGATGAGTCACTGGACCCCACCCTGGCTTGGATGAAGTGGCCTCAAAGTGTCTGCCTTGCTTAACTCGGGATACAC
TCAGTAGCCAACCCACTGATGACTGCTTTGCCTCCCTGGATTGCTAACATTGAAAGGGTGCCCCAGCTGCAAGTTACTG
AAGGCTGATCCTCAGCCTAACACATCACTATGCAATTGTATACTTCTGGCTCACTGTGCACTGGATGACTAGGGACAGA
TGAAAACTCCCAGCCTGCAATGATAAACTCACTGAGCTACCAGTCAGACAACAGGCACAGTGCAAGTCCTCCTGACTCA
TTCACTTTGATCCCGGAGCGCAAAAGATGGAATTCCATTGTCAAGGTGGGATTATCCTGTCCCTGACCCCAACTCTGAC
CCTGTTCCTACCTCTTGACCCAACAAGGTGTGGCATTTTCTGCTTCTGACTCACAGCTTCCCCAATCCTGGTTATCAGG
TCAGGTCAGTAGGTCAAAGGCATTGTCTCCCCCCACCCCCCCAAAAAAAGATCGGGGGTGGGGAGAGAGGGAGGGGGTG
GGTCCAGGAATGGAAGGACACAGAGCAGAGCAATCTTCCTGTGCCCAGGCAAGTCATTTACTGTCTCTGACCATTTCAG
TTCAGCAGTTTGAGGACTTGGTCAGGTACACACACACACATACACACACACACACACACAGAGAGAGAGAGAGAGAGAG
AACATGAGAGAACAAGAGAGAACATGATAAAACATGTCAAATAGCTGTTTTCAATTAAAATAATTTATGAACCACCTTT
TTGTTTTAACCAAGCCAGGGTCTCACTACAGAGGACTGGTTGGTCTGGAGCTTGCTATGTAGATCAGGCTGGGATTGAG
CTCAGAGATGTCCCTGCCCCTGCCACCCTCTGCTAGAATTAAAGATGTTCACCTCTTTTAGTTTTTCCTTTTTTTTTTT
TTTTTTTTTTTTTTTGAGAACGTGTTTCTCTGTGTAGCCCTGGCTGTCCTGGAATTCACTTTGTAGACCAGACTGGCCT
CGAACTCAGAAATCTGTCTGCCTCTGCCTCCCGAGTGCTGGGAGTAAAGGCGTGCAGCACCACCACACCCGGCTAACTT
GTTATTTTTTTTAAAGTATGTAGCTTGAATTTTCCTTGAACTTCTTTCCAGTCCTCCTGCCTCCACCTCCTGAGTGCTG
GGATTACAGGTTTGCGCCACGGCACCTGCTTTTGAGAGGTGCTGTATCTTTAGCTCTCCTCTTACTTCCAAGGCAGAAC
AGCGCAGATCCCGGCCTCCTCTCCCTATGATCAGCGTAGTATATGGAACCCGGTCCAATTCCCAGGCATCCGCAATTCC
CCAGGATGGCCGGAGCCTCCCGTCCTGGGAGTAGATGATATGAATGAATCCTGGGAACCTCTATCCTTACATCTTTCAC
CTTCGTTGTGTGTGTATAAGTGTTTTGCCTGCATGCATATATGGGTGCCACATGTGTGCCTGTTCCCCTCGGAGGTCAT
AGTAAGATGTTAGATCTCCTGGAACTGGAATTACAAATGGTTGTGTGCCTCCATGTGGGTGCCAGGAAGTGAACCCGAG
TCTACTGCGAGAGCAGCAGCACCACTGAGCCATCTCTCCAGCCTGTCTTTCCTGCATTTTATTTAGTGTTATGCTTTGC
CTTTTGGGCTTTTGCTGGACCCTGACTACTCTTCTCATATATATTCATCCATGAGATTCACAGGGACACAGTTAAAGAT
TCATTGTGAGCAGAAGAGTCATGATTTTGAGGTTAGCCTGGGCTACGTAGTGAGTTGGAGGCCAACTGCAGGGGTCAGT
TCTCTCTTTTCACTACATGGACCCCAAGGATCGAGCTCAGGTTACCAGCTTGGCACCAAGTGCTTTTACCCTCTGGCCC
ATCTTACCCACTCACCCATCTTACCCACTGGCCCTCTTTAAACATTAAGAGACAGAGTCTCACTATGTAGAGCAGGCTG
GTTGTCAAACACTCTGTGTAGCCAAGGCTGACTCCAAACTTAAGCACTTCCTAACAATCCTAGATGTCACCAGAAAATT
TCCACCACTACCACTGCTAGTAATGTTCCAAAACCAGCCTATTATCCTAGACAGCAGTGTTGTACATATGGACAGTGCA
CACCATTGCATCTATGTGTCCAGAAAAACGTACAAAGGTCCCACAGCTCCAGGACACACTGTATCCATCTTTGGATCTT
CACATATGTCTTTCTCCCTTCTAAGAATATCATGCCCTGCTCCTACCCACCACCGGTGGAAGGATACCATTCCACTTAC
TTATTTACTTATATTTTTGAGACAGGGTCTATTTAGCCGTGGCTGTCTTGGAATTCTCTATATAGACCAGGCTGGCCTC
AAACTCACAGACAACTGCCTGTATTTGCCTCCATCTACTAAGATTGCAGGCATGCCTCCCAGTTCACTGCCCTGCCGTG
GCTTTAAAATGGTTCCATTCTCTGTCTCTCCCTTCCTCCTCTTTGCTAAAGCATTGAAAAATTCTCTATCATAAACAGC
TTTGGGAGAAGACTGTTATAATGAACAGTTTTGGGGGGCCATAGCCAAGCTGGAACGTGGACTTCTGTTCAAAATGTAA
TAAGACTTTCAGTCTGGCCCTAGTGGAGTATTGAATCAGGTGTTCAAGTGTGAGTCCTGGGAAGAGCAGGGCCCTGTCC
CGTCCCCTCCTCAGATGGGAGGTTAGGCTCCGAAGCTTTAGCTGCCTTCCCTTCCCAAGTGCCTGTACCCTGGTCCCCA
TCTGTTCTATCCATATTTGATGTAAGGAAGAACAGGATTACAGCACAGCCATTGTCGGACATCAAAGTAGGCTCACTTT
GTCTGCCATAAAATATGTTAATCATTGAAATGACAAGTTTTGCCAGAGAGAATGTGTTTGTGTGGCTACCAGGCAGATG
GGAGCAGGAGGCCCTACCTCACTCAGCTCTGCCTTCAAGGCATAGATGTGACAGTGTTTGCAGGATGAACAAGGTGATC
GGAGGTAATTGGAATGATGGGTGATTTGGGCAAGAGTAGCCATAGTTCTTGCTTATGTGTAAGACTCATGCTCAAAAAG
GCAGCAACATTAAGAGGGAGTTGTGATGGCAACCATTCTTAACTTCAGTACACAGCAGTCTGAGGCAAGATAATGGAAG
TCAAGACCAGCCCAACTCTATAGTAATAGTCTCTCTTCAACAACAACAACAACAATATCAACAACAGGAGCCAGGGTAG
GTGGCAAATGCCTGTAATCCTAGCATTTAGAGGGGAGGGGCAGAAAAATCAGGAGATCAAGGCACGTGCCATCTGCAGA
GTGAGTTTGAGGCCACTCCGGGCTATGTGGGACTCTATTTCAACAAAGCAAAACAAATAATGAGGAAAAAGAAGGGGAT
ACAGAGATGGCTCAGTGGCTACAAGTGTTTGTTAACCTTTAAGAAGCCTGAAGTTCAGTTCCCGGAAGCCATGATTTAT
AACTCGAGCTGTAGGGGATCTGACACACTCCAGTGGTAGGGGATCCGATGCTCTCTTCTGGTTTCTTCGGGCACCAGCA
CACAAGTGACAGACACAGAAATGCACACATACCCAAAAACAAAAAAATGATAGAACAACAAGAGAAGGACCTGATGGGC
CAAATGTCTCTAATGTCAAAAGTTGTCCATCCACAGAGCTGCTGTCTGGAGTAAAACTGTCAGGGGCATCTAATACTTC
CTAAAGTCCATGGTGAAATGTTGGCAATGTGTAAAACTGGACATATCATTTCAAATGCCAAGCCTCAGTGTGGTGGTGC
AAGCTCCTAAGATGTGGAACAAGAGGATAAGGAATTTAAGGTCAGCTTGAGCTACATAGCCCATGAAAAGCCAGCTTTA
CCTACATAAGACTATTTGGCATAGAGGAGGGAGAAGAAAAAAAGGCCCGGAGACATTAGGGGACTTTGTGTATGTGGGT
GGTTGCTCACTCTGCCCACATGTTGGGGGGCAATGGGAACTGAGCCCCCAGCCTGGTCTACTGAGCTGGAAAATCCAGC
CTCACACACATCCCTTACACCCGCACACGGCCCTGAACCACCTGTCTTGTCTACATTCCACTTAGTATGACATCATATG
TGGTTTTCCATGTTGCTACAAAGTCTTTGAAGCCTTCCCTTTACACTTAAAGGCCCCACACTAGTCCAACTAACATCTT
CATACCATTCCACTGGTGTGAGTATTTGTGTTCCTCCACCCCCTTTTTCGAATTACAAAGAATTCTGCAGGTAACAAGT
TATATCAGGTTGGCTCAGAGGGAGGGAGGCCATCTGCATTTGGCATCTCCTTTCTGTTCCTTTCTCGCCCCTCCTTGCT
AACCTCCAGGCTTCTACCTGGAGGCCCTCAAAGAGCCTGGTTACTCTGTCAGGGAAGAAGGGAGCTATTGTGTCAGAGG
GTGGAGAGGGTGAGTGGGAGAAAACCAACTCCCTTGGAATTCACATGCCATTCAGGCTGAGATAATCACCTTTAAGCAG
TTTAGCCAGATCTCATCAATCTTAAATAGCTTTGACAGCCAGAAAAATTAAGACCATCCCAGGATGCTTACCAGTTTTG
AGAGATTATAAATCCTTGATCACAAAGGGACCAGGTGAAGGATTCGTAAATCCTGTCAAGAATTTTAGCCTTCAAAGCT
GTGTGTAGTGTTACATGCCTGTAATTCCAGCAGTCAAGGGGGTGGAGACAAGAGAATCAGCCGTTCAAGGCTATGGTTA
CCTACATAGCAGCTCCTAGCTAGGCCAGGCTACAGGAGACCTCTCTGAAGAAGCAGAAATAAAATAATAATAATAATAA
TAATAATAATAATAATAATAATAATAATAAAATGAACCTTTATCCAAAGGGTTATAGGTAGCTGTTGAATTTATGACTT
TGTTTTTTTTTTTTTTTTTTTTTTTTTAGGCAAGGTCTCACTTAGTAGTGCAAAGTTGTCTGGAACTCATGGCTATCCT
CCTGCCTCAGCCCCCAAGGTATTTGGGATAGAAGGTATGCGGTACTGCATCCAGCTCCCACTTAACGGCTTTAAGAATG
AGGAATTGTGGGCTGGTCAGATTCTGGTCCGAAGGCACTTGGAGCAAGGGAAGGGAGGGTGGAGGAAGGAAGGTTGCCT
GCATGACACAGGATTCTACCCAGGATTACTGAGCAGGTAGGATGTAAGCAGGAATCTGGCTCCCAATGATGGGAGATTC
CATTCTTTTGGACCTGGCGAGTTAGGGGTGATCTCAGGCAAAGTTCCAGCACAGCCGTGATTTGGGGAAGGCAAGTCAA
GGGCTCCGCACTCATTGCCTGGGGCTCTTGTGGGCAGACAACTTGATCATACAGACCTGGGACCAATTGGTAAATCCCT
TACTGGTCTGACCAGCCCAGGGAACTGGGTGTTCTGGGAAGCCACAGTGGGATCTGCAGGAGAAAGGGGACAGTAGAGA
GATTCCAACATCTGTGAAGTCGACTGAACCGGAAGACTACTAATCTCCCCCCAGCAGAGGGTTCTAGACCACCCCATCC
TTCCCACTGCTTGAATTATAAGCCTACTTAGAATTCAGCTTAAAGAGTCAGATAGATTTATTTATATTTATTTATCTGT
GTTTGGTGGAGCTACAGCTCAAACCTTCCTCAGACTCCTCCTAATTCCTCCTAGCCAGGACGGCCTCTACATCCCTCCC
CCCCACTAAATTACATACCCTTTTCTTTTTGCTAGCAAGTCATCTACCTACTTTATGATGCCGGTATATTTCTGGACTA
GGGATCATCCACTTGAGCACGGTTGAACTATCAGGAGCTGTACCCTTAAAGAAACCTGACTCTTCTCCTGGAAGCCACC
AACTGTCCACAGCTCCTCAGGGTCTCATGAGCCCCCTTCCCTCCAAGCTTTGGTCTTGTGCAGATAGGAGTTGGCTGTG
TAGATTGTTGGTCTTGAGATGGGCTCCCGGCTGAATTCATAGTCTTCATTTATTTTTGTTTATTTATTTTTTTTAACCC
TGTAGCCAAAGTGGCTGGAACTCACTATGATCCCAGTTGGGTCTCAGACTCACACTTATTCTACTGCCTCAGCTTCTGG
ACGTCTGAGTGTTCAGATCTATAGGCAAGAACACCACAGTTAGCTCCTGATGATTTTAAATACAAAGGTTATTAGCAAA
CCATGGAAGGAAGAATATGGGCTATTTTTGTTTTTGTTTTTGTTTTTCGAGACAGGGTTTCTCTGTATAGCCCTTGCTG
TCCTGGAACTCACTTTATAGACCAGGCTGGCTTCGAACTCAGAAATCCGCCTGCCTTTGCCTCCCGAGTGCTAGGATTA
AAGACGTGCCCCACCACCGCCCGGCTTGACTATGGTCCTTTTGTCTTCCTTTCGGCTTTTTCGTGTCTGTGATCCTTCA
TTTACAAGGCATGATAGGGACTGCAGGATTGCACCGTTAGGATGGGGGGGGGGCACCCTGCTGAGTGATTAATTAGGTC
ACCTCTGCTCTCTCTCATTTCCCATATCCATAAAGTGTAGGCAGAAATGTTCACCTTGCACGGTACTGTGAAGATTTGA
TATGGAAGTGCTCATCTCCCAGTGGTGGAGAAATAGAGAAGGAAGGTGCTGGAGCAAAGGAAATAAACATTTAAAGCTT
TACTGCAAACCCCGGGAATGCACAGCCTAGAGGTAGGGCACTGGCCTAGAATATATCTTTGGCCATGGATGTAGTCACT
GGTGTGAGAATTAGTTGTTTTTTTTTTTGGGGGGGGGGGAAGGATTAATCTTGCCACAGTTTGAGAGGGTTACGGTCAC
CATGGCAGGGAGGGCGTGGCTGAGTGCAGGTCAGCAGGATGGTGTGGCTGGAATTCCTTATGTCCTGCCAGACCAGACA
GCATCTGCATTCTCATCCTCACCAACACCCTCTGACAGGACGAAGCGACACTCTCAAAGGCTCCCCTTCAGCATGCGCT
CAACCTGTGTCCTTGATTCCACTACTTAGAATAATAACTACTAGGCCAGCCAGACGCCTCGGCATGTAGAGGTGCTAGT
GTACGGGCCCAGGAGATAAGTGACTCCCAAGAGCTGTCCTCTGACCTCCACAGCACACTCATGCCTGCACACACACACA
CACACACACACACACACACACACACACGTGTGCACACACAAAATTAAAGTGAAAGATAACAGCTTCTGTGAATTCAGAA
ATTGCTCTGTGCCATGATATTTCAACTGTCTTTATAAATTCTTGCCATAACGCAACATGGATTAGTGGGGGCGGGGGGC
ATGTAATTGTTATCTCACTTTACTAATGGGGTTCAGAGGTTAAATAGCTTGTTCCAGGGATCCTAAAAAAGAGGCCAGA
TCTGACATGTAGCTATATTATCTAGCTATGTGATCTTGATAGTCCCAAACCATCTCTCTTCAGTATCTTAAGCCTTGCA
GTGGAAGAGGAGTCTCTGTGAGGTTTATGAAAAACATATATGAAATAATTATGCCAATGCCTGACATTTCATGGGTAGT
AACTTTTAAAAAAATAAATGGTCAATGTCTTTGTTACTGTTCTATTGCTATTACTGTGAAGAGAAACCATGACCAAGAC
AACTTATAAGAAAAAGCATTTAATTGGGGCCTTGCTTACAGTTTTAGGTTCGTCCATGGCTGCAATCATAGCAGAAGGC
ACAGAGGCAGGCAGGCAGGCAGGCAGGCAGGCAGGCAGGCAGGGAGGCAGGCATGGTGCTACAATAGTAGCTGAGAGTG
CCTTTGATCTGCAAGTTGCAGGCAGAAAGAGAGGGAGGCTGGGCCTGGTGTGGGCCATCTCCCAAAATACACTTTTTCC
AACACAGACACACATTTTACTCTTTACCAAACAGTTCCACTAACTGGGTGAGGATCTGGCCCATTCAGACCACCAAAGT
CAGTGTTATTGAAATTGGGTACCCTTCAACGATGTTGAAAACCTAACCACCCGATGCCTATAAATGTGGCTCCAGTTGA
AACCAGAGTCTTTGCTGCTGAAGAAGTAAAATTGAGGAGTCCAGGGTGGAAATGTCTATGCAAGTAGACGTGCTCACAG
AGCTTTGTTTGCTGGTGCAGGAAGGAAGGCATTGGGGATGATGCCCCAACAAGCCCCAGGGAAACTAAAGGCTGTTGGC
CAAACTATAGAAGCCCAGGGGAAGCCCTGGCATGACTGCCCACCCTTCCTGGCTGCACAGGAGCCAACCATGCTGACAC
ATCGTGTTGGAAGTGCAGCCTCAAGAGCTATCAGAAAAGGCCGCCCTGTTATTTCAAGCCACCCAAATGGCAGTGCGGC
TTTTGTTTTTGAGACAGTGTCTAGCCATATGTGGCCGGGAACTCCCTTTGTAGCCAGGTCACCTGCCTCATGCTGGCTG
TGACCAGCTTGGTTCTGCCTCCCCAGGGAAGGGATTACACTATGCATCTCTGTGTCCAGCTTTCCCACATGTTAACTGC
ATCTTTTTATTTTGCTTTTGGTTTTGTTTGAGATAAGGTCTCCTGTATAAGGTCTCCTGGTCTCCTGGGGTGACTTCAA
GCTAGCCAGGAACTAAGGGTGACCTTGAACTCCCAGCCTCTACCGCCTGAATGCTGTGGTTTGTGGACCACCATAGCCT
GATGTAAAGGCTTTTTTTTTTTTTTTTTTTTGGTTTTTCGAGACAGGGTTTCTCTGTACTGTATAGCCCTGGCTATCCT
GGAACTCACTTTGTAGACCAGGGTGGCCTCGAACTCAGAGATCCACCTGCCTCTGCCTCCCGAGTGCTGGGATTAAAGG
CGTGCACCACCACGCCCGGCTGGCTTTTTTTCTTAATGAATGGATTAAGAGAAAATAAAACATGGCTGCCATGATGGCT
CAGTAGAGGAAAAGCTTCAAAAACACAACTCAAAGACCTGAGTTCAAATCTCAGCACCCACTTGGCTACTGCATATATC
TGTAAGCCAGCAGTGTGGGGTGAGCCTGATCCTGGGGTCCCGTTAGCTAGCAAGCTTTGCTAAAATGGCAAACTTCAGG
TTCAGTGAGAGACTTTGTCACACACACACACACACACACACACACACACCAGGCCTCCAAGCTCACTGCATATACACAG
AAACCTGCCAGTGTACATGTAATTAAAAATAAATCTAAAAATATAAGATGGCGAGTGATGATAGAGGAAGCTAGCCACG
CCCTCCTCTGATCCCTGAACCCGTGCACTCACATATACATCTGTAACCCCCAAAATTCTGAAGAGGGGCAGGATTCTGG
AGAGACGGCTCAGCTGAGAGATTGCCTGTTGCATTACTGTGAGGACCTAAGTTTGATTCCCCAGCACCAGCCTAAAAGC
CCAGGCACGGTGGTGCGTGGTTCTAGTCATAGCAATGGAGAGGCAGAGACAAGAGGCTGCCCAGGCTCACTGCAGCTCA
GCTCAGCCTAAGGAGTGAGTCTCCAGGCCCAGGGAGGGGGGTCTTTCCCTTCCCTTCCCTTCCCTTCCCTTCCCTTCCC
TTCCCTTCCCTTCCCTTCCCTTCCCCTTCCCTTCCCTTCCCTTCCCTTCCCTTCCCTTCCCTTCCCTTCCCTTCCCTTC
CCTTCCCTTCCCTTCCCTTCCCTTCCCTTCCCTTCCCTTCCCTTCCCCTTTTCTTCCTTCCTTCCTTCCTTCCTTCCTT
CCTTCCTTCCTTCCTTCCTCCCTTCCTTCTTTCCTTTCTGGTTTTTTTTTTTTTTTTCTTTTGGAGTAGTAGTGTCACA
ACTTTATTTATGGTGCCTGTGCTTTGTCTCAAAAATACCTTCTCCCCCTCCTCAGACAATGGATGGGAAGGAGGCAGCA
AAAAATGGAAGACATCCCTCCCTTACTGCACACGGATCCTATATCCAGGCCCACCTAGCAGAGGCCAGTGGGACTCTTG
GCACATTCCTGGATCCCTGGGGAGGGGGAGACCTTTTCTTTAAGCACACACACACACACACACACACACACACACACAC
ACACACACACACACACATTTTCACAGACAGGAGCTTGGGGGTGGGGGAGAATACAATGTTAAACTATTTTATTTAGAGC
CAGGACAAACAAGTAAAGATTATGGCTATGCACAAAACAGTGAAGATTCAAAATGGGTGCTCTCTCAGCAAGGCTAGAA
ATCACCATCTCTGCTATTTGGAATTTAGGCTCCAGCCCTTGTTTTGGGGTGGATGTGAATTACATTCTAATCTACTCTG
AGAGTTTCAGGTGTGCACATCAGCCCCTCTTGAGGGTTCCCTGAGGGGAGCTGCGCCGAAGCCTGGCTCTTTTGACAGG
ATCCGTCTTACAGGATGGACGGACAGACGTGATGACGTGGTACCAGTCAGCTTTGCAGGGGTCCTGCCTGCCGTCTGTG
CTCCCCCTGGGTTACTTCTGACTGCCCTCATGCCACACTGGCCCGGGAGAACCGTCTAATTTCAAGCATGTCTTTTGGG
TGCTGAGTGAATACGTCACACTATGATGGTGAAGTTTCAGATACCGGGGGTTACTACTGGAAGATTTTGAGAGACTCTC
ATAAACAAAACATCTGCCTGACTTGTAGGCACCGTGTTGTTGTGAGCCTTATTCGGTACACAGAGTCCCTTCTGCTGTA
CTGGAGAGATCCTTACTAGCTGTTTAAGCAACCCAGGTAGGCACCTCAGGCCTTGAGGGAAATTAGGTGCATGGAGCCG
GGAGAGGGTCTCATGGGCCTCGTGACCCTTGGGGATTATGGTGGAAACTTAGAAAACAATTACAGTGGAGCAAGCTTCT
CTTGCTGCAGCTAGGGAGGCCCGCCCAGTTCTGCCCTTCCAGGAACATGAGAAACCACGCTTCCTCCCTCCGGTTGGAA
AGGAATGCTTTAAACAGATGATTATCTTAGCTGCTCCAGACAGAGTGAGAACAACCCACTATGCCCTCTCTATTATCTG
ATGGGAGACCAATCTATCTACATAAGATGTCTATTATTATGTAACAAATTAGCCCCAAATTTAGTGCCATTAAAAAAAA
AAACCAACAACAGATATTCACTGTCTCCTAGTTTCTGACGGTCAGCTTAGGTGAGTGGTCCAGCTTGGGGGTTTCCTAT
GGGACCAGAGTCAGTAGGGACTCAATCAATCCAAGTCTCAGCTGGGCTGGAGAACTCACTTCTGAGATGGCTTCCAGTG
GGGCTGTTGGCTGGAGGCCTCTTCCACCCAGGCTTGTCTGGATGCTTCTGTTCCATACCACAGGGGTAAGGTGACCAAC
CATCTGTGTTGGTCCAGGACTGGGGCTTCTCAGGATGTGAGAGTTTCTATGATAAAAGCAGGATGGTCCTAGTAAGATC
AAGACTAGTTGGTCACCCTCTATGAGCTTCTCCATGAAACTGTTCACAGCATGAGATGGTCTGTAACCGTGACTAAGAC
TGAAAGTATTTCATAGTCTAATCTTGGAAGGAGCATACCATGCATCTAAATGCATTTTTCTGGTACATTCTATTGGCCA
CACAGACTGGCTTTGATAAGAAATGCAAAGGTTAGACTGGAGAGATGGCTCAGCAGTTAAGAGCACTGACTGCTCTTCT
GAAAGGTCCTGAGTTCAATTCCCAGCAATCACATGGTGGCTCACAACCATCTGTAATGGGATCTGAGGCACTCTTGTGT
GTGTGTGTGAAGACAGCTACAGTGTACTTGTATAAACAATAATAGATAATATTTTTTAAAAAAGAAGTGCAAAGGTCTA
GAAAAAGGGTATGACTATCAAGAGGTTGGGTTCATAAGGACCATGTTTGAGGCTGGCCTCCATATTACTCTAGAATTAG
ATTCCTTTGTGTGTGTCTCTGTGTGTGCTTGTTTGTATAGTTCTTATGTATCATGCATGATGGGGTAGGTTGGCCTCAA
ACTTATGATGGCTTTGAACTCACTTCCGAGATGGCTTCCGGTGGGGCTGTTGGGGCTCACAGATGGTTGTGAGCCTCCT
CCACCTCCCTCATGCTGAGATTACAGGTGGGTGTCACCAGACCTGTCTGGAACTATATTTCAAACATCTGTATTTGCAC
ATCATTTCTCTGTGTTAAGAGGTCTCCAGTGTGGTGACCCTTCTGTTCCAGAATAGGTTGTTTAAAGCTGCAGGCATCT
GAGGCTCAATGAGAGTGGGATCCAAAACTCCCTCGGTAGGCTGGCAGCTGGTGCTGTGGTCACAGGAGAGAGCCGTCTA
TCTCCACCTGCAGCCTTCCCATACAGAACAGCTGTGTTCTGGTAGCTCTCTCTGAAGATGCAGAAGCTGCCTGGCTTCC
TGGGATCAGATAGCCTTCAATACATCACCATGCATCCTACTCCCAAAGCTGACTGACACAGGCCAGCCCTGACACAGGG
CAGGGGAAGTTAACACTGCTTTGGGCAATGAGATGGGTATGTATACTAGAAGAGTTACATGAGTCTACCTACTCTCTAA
AGCTGAGACTTTGGGGTCTCTCCTGAGAAATGCACAAAATCCTCAAGACAATGGGTGATCCTTACAAAATCAGTGTTTA
TTACTATAGTTCATTCATTCATTCATATATATATATATATATATATATATATATATATATATATATATCCTCTCTCTCT
CATTGATTTCCCCTATGAATCACACGGGGAAGGTGCTCTTTTCATCCGGTGCATGGGGAAACTGCATTTCCAGGAGATT
AACTTGCCCAAGGCCAGAATGCTAAAGCTGAGTCCAGCTCCAAATCAGTGTTTTGAAGCAGCACTCCCTATTTTTAATT
TTAGTCAACTTTTCACTTAATTAATTATAAAGAATTTCCTTTTGTAAATGGAATTATAGGGTGAATATATGAGGTTAGA
GTATTCGAGTTTAGGAATGAGAGAGGAGTGTTAATTTAATATTGTTTTGTTTTGTCTTTTTTAATTTTTATTTTGATTT
GTTTGTTTGTTTGTTTTGTGAGGAGATCGAGACAGGGTTTCTCTGTGAGGCTTTGGCTGGTTTCTATCTGCCTGCCTCT
GCCTCCTAGTGCTGGCATTACAGGTATATGCCATCATTGCTGATGTGTGTGTGTATTGTTGTTGTTGTTGTTGCTTGGT
TTTATGAGACAGGGTTGATCTGTGTAGCCCTGGCTGTCCTGGAGCTTGCTCTGTAGACCAGGCTGGCTTCTGATTCATA
GCAATCCACCTATCTCTCTGCTACTGAGAGCTGGGACTAACGGTGTGTGTCATCTCTGCCAGGCTTATGATGTTTGTTG
TTGTGTGTGTGTGTGTGTGTGTGTGTGTGTGTGAGAGAGAGAGAGAGAGAGAGAGAGAGAGAGAGAGAGAGAGAGAGAA
GGAGAGAGAGACAGAAGAAGTTGGGTGGGTCTGAAGGTTGGAAGTGTAAGGGGCCCCTCAGAGTCCCTACAGAACACAC
CCAGATACCCAACGCTCAGCACAAAGGTAATTTATTACCCCTAGGGACAAGAGGTGGGAAGGGCAGGAGGAGGAGGGGG
GTGTACTTCCCCTGGGTTAGACAAAGACAGACAGCAAGTGGTATTTTGTTGTTGATGTTGTTGTTTTCTGCTTTTTGCT
TTGTTTAACAGGAGTGAATTTTTAAGGAGAAAAAAGGGAGAGTCTGTGATCGGGTGAGTTGGTCAGGCCAGTCAGTGTA
GCCAGCCAAACAATGAATTTAGACTGCTGGATCTTGGTGTTCTGTCTCAGGAAGGAGTCAGTGGCCAAATAAGAGACTA
GACCTTGGGGGCTAGCCTTAAAAATATAATCTAACAGTTTAGCAAGGGTGAGGGAAAGGGAAATGGCAAAGACTGCTGG
GGTCACGTTTGCGATGCTCAGGTCTATTAGCCCTTCAAGGAGGACCTAAGAGGGGTTACGGAGAAGGGAAAGAATACGG
CAAAAACATATTGTAGGATTTTTTTTTTAATTAACAAATGGGGGCTGGAGTGGCTCAGTGGTTAGAGCATTTATTGTTC
TTGCGGAGGACCTGGGTTTGATTCCTAGTATTCACACGGTGGTTCACAACAATCTATTGCTTTAGATCCAGAAGATCCG
AAGATCTCATTTGGTCTCTACCAGCACCAGGCATACATGTGGTACACACATATTTTTAAAGCAAAATACCCATAAAATA
AAATTAAAAAAAAATATTTTTAAGCAGCAAATAATTAGTGGATTTCGAGACGAGGTCTTGCTATGTAACACAGGCTGGC
CTGGAGTTCAATGTATAGCTCAGACTACACCAACCTTCCCTCTGGAAGGCTGAGATTACAGACACAGGCACCTTGCTCA
GCCCCTCTTTAGGACCTCTTGCTATGAGAATTCTACCTTTCCAGTCCCGGCCACTGCACTGTGGGTGGTCCACACACAC
AGGAGGCCAGGGGTAGACATGCCTCTGCACTGTCCACATCTGAGCTGACAACCATCAGCAATTAGCAGCCGTCTGAAGA
GCCACACTGGAGGTCCCAGTCTAGGTCAAGTCCCCAGGTGCTGCAGCCCCTACTGACATCTCTCAGCCTGCGCAGACGA
GCTGCCAGCAAAAAGAACAATAAAATGGCAGACAAGGTTTCGGCAGGAATTTTTATACACAATTACAACAGGGTAGTTG
TACTTCCTTGGGTGAACCCCAGCAGATCCTGAAAAGGGGGTTCCTGCCCTGGGAAGTGTTTGAAGTAGGGAACCTTTCA
ACGTACGGGCGAGGCCAGTGCACATAAGAGGCTGCTGGGTCAACCTCTGACATTTCTCCTTGTTTCCTGACCCAGCTGA
GAAGACTGCATGGAGTCACAAGACTGGGAGGAGAAAGCGTGTGTGAACGAATTAACAGAAATCCGGGCAAGAGTCCCAA
AGTAAAATAATGTTGATTCTCAGCACAAAGGAGGCTTTGCTGCTTGGTGGTTGAACTCACAGAAACAGTCATTGCCTGC
TTGTGACAGATTTACTATTTTTGAATTTTTCTAATTTCAGGGGGAAAAATGACCATAATAAGCGCCATTTAAACACTGG
GAGCCCTTTGGAGATGTCTAAACAGTGTGTACAGTTCCTGAGGGGGCACAAGGGGTTGCTTGTCCTTGCAGATTAGTCT
ACACACAGTTTTGTAGACAGGGGGTGGTGCCAAGATGACCTTGAGTTAGCCTTAGTTGGCATTGGATCTCGACTCCTTC
AGAGTATGAAGTGGGCAGAGGGGGGCTCCAGTGTCCCGGGAAGAGAGTGGCTCTGTGGCCTGCACCAGTCACATTCATA
TGCTCTCCACCCCCTCACCTCTCAGCCTTTCCCAGTCGAGTGTCTCTCAAATTTTAGGGCATCCATGCCATCTGGGAAG
GATGTAAAAGATGTAAATTGCGGAGTTCTCCCTGAGAAGGGTCCAGTTCCCCATGTGCGGGGTCAGTTTTCTGCTGTTT
TCTTTGGATGGCAACCCTGCAATTCTGTGAACCAGGTTGATTTCACAGCTTCTTTGCTCGTAGTTTGTTTTCTGCTTTG
TCCTATTTTGCCCACTGGCATACAAATCCTCAAATGTGTCCCCTCCCCCCTTCCTCACTCTCTCAAGTGCTGGGATTAT
GGCCTTGAATCACCCAGCCCAGTTTATAGAACTCTGCAGCTGAGATCGGAGTCTGGCTGTGCTGACACACATCCGTGAT
CCCTGTGCTCAGGAGGTTGAGACAGTTACATGGTGAAACCCAGTCCCAAACACAAAGGGACGTGCATAGCAGACGAGAA
GACCTGATCCCACCTTGAAGCAGATGGGATCTTTGTAGATCAACAAAGTCAACCGGAACAAGAGTTTATTTTATTTTGT
CCCAGAGCACTTCTCAAAAATTCTGTCACCTCCAGGAGAGCAGAGTTTTTGTTTGTTTTGTTTTTTATGTTTCACGATA
GTGGATTTCTGAGAAAGATCTCATTAGAAGAAAAAGATTGCAGGGCTATGATTTAGTGCTTGCCTGGCAGCTTAAAACT
GGTGTCCAATCTCCAGGACCATGGAAAAGAAAATAATAGAAAAGTAAAACAAGATAAAAACAAGTCCAGGGTAGTGGCT
CCCCCTACCCCCACCACCCTCGTTTTTCTCTTTTGAGGCAGGTTCTCTAGCCAGGTGACGTCAGACTCACTCACTATGT
AGCAGAGGTTGACCTTGAACTTCTGATCCTTCTGCCTCCGATTTCCACAAGCTGGGATTATAGACTTGCACCAGCACAC
CTGGCTTTATATGGTTCTAGAGATGGAACCCAGGGTCCCTGGGCATGGTAAGCAGGCACTCTGCCAGCTAAGCTACACA
TACACACACACACACACACACACACACACACACACACACACACACACACACAGCTCAGGGATACTCTACAGCTTCGGGG
AGGCAGCA

An exemplary human Ky DNA is provided below: GenBank Accession No.

NC_000003.11 (GI224589815: c134378285-134311000)
(SEQ ID NO: 302)
CAGCCCACCTTTGGGCTATTGTGAAGACTATGTGAGTTCATGGAAATGGAAATGCAGAATTCTGCACAGA
AGGAGGTGACTGGTATGGAGCAGACACTGAAGCCTCACCTGCAGAGGACACCTTGTTTACGTCAGAGAGA
GGGGCGGATCGAGGAATATGCAAGGGGCATTTCAATAGCTGGGTAATCAACACTTGGCTAGGTTAAAGAG
GTAATCTCTCCATGCGCTCTCATGTGCCACTGGATACCTCTCTTGGCATTGCCCAGGATATTTTGGAAGT
GGTGGTGAAAGACAGATTGATCTTCATCATCTCCCTCCCCAGCAAAGGGCCAAGAATGCCATGGAGTGTA
AAGACTCCAAAATTCAAAGTACCCGAAATTTGGAAAGCAGTGCTTCCCACACTTGCACATTATTATATGC
AAGAAGATGAGATTTTAATGAGCTCTGGACAGAAAAATATGGAAAATCAATTAAGGTGTCATGAACAGAA
ATACTTTGAGAAAGGGAGTGGCTGACAAACTGATCAGGAAGAGACATACCAGAAAGGTTAAGAATGAAAA
TTGCTCTTAACAGCATGAATCGTGGCTTATGACTCTGATCGGAGCAGGGAGCAGGGCTGGTGTCTGCATG
GGACTGAGCAGCGCTGTCTCAGAACCAAGCTCTCCCCAAATTCATGCTCCAGATGGTTCTGTCAATGTGG
AGGATGGTGCCTTCAGAGCTTCTTGTCCTCGTTTAACCTTCTTCTTCGTTCCCTGTAGGTCTGTGAGACA
GACACAGAAGAGCAAATGCTACACATCTCTGGAAAGCTCAGTTCTACCTCCAGGCTAAAGGAAGAAGTCT
CAGCTTGCTGGCAGCATTAATTCTGTCCAATTTGATTCAAACATTTGTTGAGCACTTTCTGTGTTCAACA
AACATAGGCACAAAGTGTTGGGGGATTCAGGAATGAGTAAGACCATGACCTGGCCCCCTGGCCCTCAAAG
AGCTTCAAGTAATCAAAGCTATGACCATGACTCTGCCACAGACTTTGCTACGAAATACAACAGATCTGAG
GTAGGTTGGAAGAGTATTTTTTTTCCAACCAATATTTAAATATTTGTGCCACTAAACTAGGAAAAAGGAA
CTAAGGATATGAATTTTTTAAACACACACATGCACACACAATACACACACACAAGGTAAATTTATGCTTA
AAAATTGTTCAATCTCCATGGTAATAAAAATGAAAATTAGAAAGGGTTACCATGTATCAGCCGGGCGCAG
TGGCTCATGCCTGTAATCCCAGCACTTTGGGAGGCTGAGGCGGGCAGATCACAAGGTCAGGAGATCGAGA
CTATCCTGGCTAACACAGTGAAACCCCGTCTCTACTAAAAATACAAAAAATTAGCCGGGCGTGGTGGCAC
ACACCTGTAGCCCCAGCTACTCCGGAGGCTGAGTCAGGAAAATCGCTTGAACCTGGGAGGCAGAGGTTGC
AGTGAGCCAAGATCGCGCCACTGCACTCCAGCCTGGGTGACAGAACGAGACTTCATCTCAAAAAAATAAA
TAAATAAAATAAAAAGAAAAGGTTACCATATATCAATTATCATAATGGCAAAGGTTGATGTACCAATAGT
TTTCAATGCTTGTGATAGTGTATGGAGAGGTTGACATACACAGCTAATGAGTGTAAATAGGCACAGTCTT
TCTGGAAGGCAATTTGACATTTCATATATATATATCTCAATGTTTAAAATAGTCATAGTCTTTGACTCAG
TAATTCCACTTCCAAGAATCTATTCTAAGGAAATAACCAGAAATGTAGTCAAAGATATTTATTATCACAT
TATTTATAAAAGTAGACAATGCAAAACAAACTAAAAGTCCAACAATGAGAAAATAGAAAAATGGATTGTG
GCACGTCAGTATAAAAAGTGACTAAGCAATCTTTTAAATTAGCTTTTGAAGAGTTTTTAGTAAACTATTC
ATTACAGGTTGAGTGAAAAAAAATCATGATACCAAACTATATATGGTGCAATACCAATTATACTTTTTAA
ATAAAAATATTCATTTTTATTTCACACCTATTGATATGGTTTGGCTGTGTCTCCGCCAAAATCTCAAGTT
GAATTGTATCTCCCAGAATTCCTGTGTGTTGTGGGAGGGACCCAGAGGAAGGCAATTGAATCATGGGGGC
CAGTCTTTCCCATACTGTTCTCCTGATAGTGAATAAGTCTCACAAGATCTGATGAGTTTATCAGGGGTTT
CCGCTTTTGCTTCTTCCTCATTGCCTTTCGCTGCCGCCATGTAAGAGTCTTTTGCCTCCCATCATGATTC
TGAGGGCTCCCCAACTACATAGAACTGTAAGTCCAATTAAACCTCTTTTTCTTCCCAGTTTCAGGTATGT
CTTTGTCAGCAGCATAAAAACAGACTAACACACCTATATATAATAAATGTTCACTTATTATACTCTATGT
TGTAGTCATAATGAAAGGAAATACTAAATAACAAAATGTTTAGAAGTGATTATCTCTAGATGGTGGGATG
ACGGGATTTAGGTGGCTTTATTTTCTTCTTTATATAGTTCTTGCACTTTCTAAATTGTTAGTAATGAGCC
TGAATTTGACAGAGCATCTTGTCTCCTGATGGGACTTTATGACCCAGCTCACCCAGATGGGTCGCCTGAG
TCTGACAACTGAGCTGGGGCGTGGCCCTTCAGCTTAGGTGGAGGCTCTGTGTCTCCCCTGCCTGTCCTCT
CCATCTGAGCCCAGTTCCCCACTTCACCCAGGCACTCTGCCCCACACTACCCCAAAATATCCTCCTTTTC
TTACACAGCATCTGTATGTGGTGCTTGACTGTCTCTTCCTATCATCACCACCCTTGCCTAGCATCTTCTC
CAGAAGGCCAGTTTCGGGTGACAGTGGTTTTGATCATGTCTAAGGCAACCGTGGGACCTGCACCCTCACA
CTTGTGAACAGTCCCTGCTCTTCCATCCCTGCTGACCAGGATCAGCATCGCCACTCAGATGGGCAGCTGA
AGCTTCACCCACAGCAGACTGGCTTTGGCTGCTCTTCTGCTCCTCTCAGTGCTCTTCCCCTGCACCATAG
CACCAACCAGACTGGCCTGCTTTCACTTCCTGAGACACGCGTTTTTCTCTTTCACCTCCATCACTCCTTC
CAGCTCCTCCTATTTCCTGCTCAGGCTCCTTCTTGCCTGGAAAGCCTGTCCCTCTCTCCTACTCATTCAT
CCCTGAGGACACTGTTTGGGGATGTAGCCTTCCTGGCCCCTCCTCTCTGGGAGGACAGATGCCTCTCCCC
TGTCCTTACTCTTTCATTTCATCCTATGGCATTGCACATAGTGAGGTCTCAAGAACAAGCCGTCATCTGT
GCTGGGAAACGTCTTCATGGCAAACCTAGCAGAGGTGGAAAGCTCTCTTTACAGTGTAACCATCCTCACC
CCAGAATGGTATGGAACTATCACAACACACTTCCTCTCCATCCCTGGCAGGGCTACTGCTCCTGAAGACT
GCTGAATGCTCCCAGCCACCAACTTTTTACTACTGCCACTGCCACCAGTTCTCTCTCCACCCAAAACAAC
TGATGCCACAGAGCCTGTAAAATCTGTCCCCAAAGGCCCAGCTCAGGGCTTGAGCTCAAAGAATTTTTCC
AAGAACCTCAACAGAGGTCTTGAAACCCCGAGTCAAGGTGTGTTCATCGTGATTGTTAAGACCATCTTCC
CCATAGGCTGACAGCTGATCTTTTCCTTGGCTCGGGCACTATGCAGCCTCAGCTCATGGGCATGTTATGG
CCATATAGGGTAGGGGGGCAGGTGTTGTTCAGCACATGAGCTTTGGAGCATACAGAGTTGGGTGGGGTGT
CAAGGGTTGGGTGTCCCCTGCTACCTGTAAGACACATGTGAGACCTTGGGTAGGTCACATTAGGTTTCTA
AACCTCTGTTTTCTCATCTATAAAATGGAGATAATGCAAAGGTTTATCCTACTGAATGCACACAGAGAGT
TCACAAGTGCCATCAGGGTGGGAAGGAAGCAGAACTGTGCTGTGCCGTAACTCCTGCCAAACAGGCTGCA
GCCGCACAGATCTGACATCAGTTCTTCAAGGAATGGATTCTGTGGCTCACTGAGGTTTTCTGTGTTTGAG
AACAGTGGCCACAGAGCAGTGATATGAGAAGGAAGGCAACACGGAGGTCAGAGATGGCTCTGGTTCCTTC
TATACTCCATCTCCAACTGTGAAACCACATCCATGAGACCTTCCCCAGAGCAGGACCCAGCAGGGGGTCT
GGCTGGAATACAGGAAGTTGCCATAGCTATAACTACCCATAGCTAAGTACTTTATCTCATTTAATCCCAG
CAACACCCTCACCCACTTGACAGATGAGGAAAACTGAGGCTCGGAGAGGTTAAGTACATTGTTCACTATC
ACCCTACTGGGAGACAGTGTCTGGAAACAGGTTTGCCTGGCATAACTGCTCCAGAGGGGATTGATATGAT
TTCCAAATTTGGTGGGTGTGGGACAAGAGCAGGGGTGAGCTTCCAAATTAACTATTGGGATTGTAAAAAG
AGGCACCACAGAGAACTGGCATTCCCCAGAAGCTCCAAGGACTGGGGTACCCAGGAAGGCTTCAAAGTAG
AGGGGAGGGAATGGCTCCAGTTCCTTCTATACCCGCCCTCCAACTGTGAAACCACATTCATGAGACCCTC
CCCACAGGGTCTTGAAGGATGCTTAAACTGTACTTGCCAGGGAAGTCCAAAGGGGTTTTCCAGGCAGAAA
TCATAGCAATAGCAAAGGCTCAGAAGCAGAATCAAGCTAAGCCAGGCCAGTCAAGAGGCCATGGGAAGCC
TACACTGGCTGAGGAAAGGTTTCTGGGAAAGGTATCCAGTATCCCATGTAAGGATTCACTCATCTCAGGG
TAGGCTGAGGGAGTTAGGAGACTGGAACTTGGTCTGTTACTCTTCACCAACTCACAGAACTCTCTGTGTG
CCTCTTCCCCACGTCATCAATGGAGAAAATTCCCTGCCAAAAGCCTTTATTGCATTGGCTTACTTACTCT
ATCTCTATAGAGAAGAGAGAAACTTGCCTCTTGGAATCCCAAGAGTGGTAAAAACAGCTATGACTGATGG
TCCCAGCAAAACTACCATTTCTGAGGGAAAACAGTGGGGGTCAAAACACACTCCTCTCTGGTGACAGCTT
CAACTGCCAGGAGCAAGCCCTGCCCCTGGTGAACCCTCCTTATCTCCAAGACAGCAGAACCAACTCAGGG
CCCAGAACCATTCAGACAACTGACAAATCTCACCTTCTAGCAGAGGAGTATCGGATGCAGGGCTGCTGAC
AGCTGGATGTGTCTAAGTGTTGAATTTGACAAAGGTGACAATACAACTATTTTGACTGTCCTTTGTCTTT
CTGTATCCTGGAATTCCCACTCATTTTGATTAAAATAAAGTTTTGCTCAAGAATGGAGAAAGCCCCTGGT
TTGTCAGCTACTAAGACAAGGATGCAGATTAATTATTTAGGTCGGAATACAGAGAGCCTTGCATCTTTTT
TATTCAAGATGGTAGCAGGATGGAGGGGCTAGTATGGTGTGTTTGGGGTGTAGAGGACATGGTTTCTGTT
GAAGCATGTGTGGGTTTGGTAGGGAAAAAGGAAGAGATGGGTCTGGAGATTTGCAGGCTGAGCGTTCTGT
TATTTGTCCCGTTGATTGTGTGAGCAGCTGGAGGGAGGATTTGAGCAGAAGGGATACAGCCTCTACTTGA
ATGATATCCCAGCCACCTCAGTGTTCTCTTGTCTCTCCTTCCCCTTTCCTGATACATCCAAATGCCTTCA
GCCACATCACCTTCCCCATGGTGTCTCCCTCTCAGCCCAAGAATGGATAGTGGGGAAGAGGGGAAGCAGC
TAAGTCAACCCAGGCCAAGGATGGGAATAGAGTTGGCATTAAGAAGAGCATCTCATCTATGCTCCTGGCA
GGCCCCAGAAGCTAGGCTGTTGGACTGATGGCCATGGGTAAACCTCTGAGAAGAGGGTCTAGGTGTCAGG
CACCATCAGGATCCAGGGGCAGCAGCATCTCCCAGAGCAGTCGAGGTGACTCCAATTTCAGGAGTTTGCT
CCAAAGCGGTGTTCACACTGCAATTCTTTCCAAAGGAAACATTGGTCAATGTCTGGAGACATTCTAAGTT
CTCACAATAGGGAAGTGAATGTGATTGGCATCTAATGCGTAGAGGCCAGGGATGCTGCTAGACATCCCAC
AGTGTACAGGAAAGCCCCACCAAAGAGAATTATCTGGCTCAAAATGTCAATAGCGCCAAGGATGAGAGGC
CCTGCTCCGAAGCTGTCTTCAGACATAGCAGAGAGACAGGAGGCCTCCACACCATTTCCCCAGACCCCTG
GCAGCACATTCCCAGGTCAGAATATCCTTCTCCAGAGGCAGTTCTGGGAGGGGGCACCAGAGTTTGCGAG
TTCCTAGCAATGACACTTTCCAGCAGGAGAGCAAGGGTTGGGAGATGGGAATGCAAGTGTAAGGAAAGGG
GACTTCAGCTCCCCCCACCATCACCAACACTCAGACCCATCCGGCAACCAAGGGTGCTGTACACAGGCTT
CTAGTTGATTTCAGCACTTCAGACAACCTGACACTTGAAGGAGTTAGACAACACTGACTGGCCCAGGGTA
GTTGGGGGGACTCTGGGACCTGAGATCTGCCTCAGCAATCTCCAACACCCCAAGAGCTTTGCATAATGCA
TAGTTCTTACTTCCCCAGCCACATAGAGAGTAAGTGTTAGCCAGCCTCCCAGGTGTGCGCGCGCGCGTGT
GTGTGTGTGTGTGTGTACCTGCACACATACAGACAGGACAAGACTCACTTTAGCAGAAGGAAACCTGCCT
TGGAAGTCAGCCATCTCCACTTGCCTGTCTATGGCCTCAGCTCTCTAGGGACTCCAGTTCAACATTACTG
GAAGCCAACAACCTCTCCTTCCATCAGATGCCCAGTGGGTGAGTCAGGGGGTGGGGTGGTGGGGGGGGGC
GCTGGTAATGCAATAAGACAACAGCCTTTACACGGAACCAAACTCTGAGCCACTGTTATTGGAGGAAGAC
CTTCAGAATCTCAGAGTGTGGCTGTGACATCTCTCCTGCTTCTCTGCTCCATCAGTCACTTGTCACTCCA
TCTTTCCCTTTCCTCACCCTTAAAATGAAGTAAGAATGACAGCTACCATATGAAGACCACTTTCTATACA
CCAGGCGCCTTACATTCATGCCATTCTCACAACAGCCATGTGAGAAATTTTTATCAACCCAGTGATAAGG
GTGAGAAGGTGGAGGGAAAATGGCTGGTCCAAGGTCACACAGCCAGGAAGTGATGGAGCTGGTACTGGAA
TGCAGTCTGTGTGACACAGGCTGCTCCAAGGAGGATCTGGGAACTCTCAGGACCATGCTACAAGGTGGGG
TCCATACACTCCAATGCGGGGAGGGCTCCCAGAGCAAAGGCCTCCATGTGGGGGTGCATTTATTAACAGT
AGAAACTCGAATTAGGGGAACGCCCCCAGCAGGGTGGGCCCGTAGTGCCCAGGGGTACCAGGCAGAAGCC
TATGTAAACACTCCTGAGACCTACAGCCAAACCCCTGCTGCTGTTGGCTGGGCAGGCCAGGCCGGGCTAA
AAATAGAAGGCTTCTCAGGTCCAGCTCCCTGTGCCCCAGCCTCCTCCTCTCCCCCTCCCTCCTTCCCCAC
AGGGCAGGAGCCAGGCTTCTAGAACGAACCGAGACTTTAAGACTTTCGTTTCAGATCAAAGAGGATCGTT
AGCTTGATACTCAATGGGCATGTTCCAAGTTCTACAGGGGCCGTAACTGGGGGGAGCTCCTGACTCCAAC
ACCCCACCCAACTCCCTCCTAGGGAGAGAGAGCTGTCAACCTCCAAAACCAACCCAGGGAAGGAGTGAGG
TCGAGGCCAAAACTTGGACAATCTCTGCACATCTTGCAATTCAATCTGGAGCCCCTTTGCTGTCTTTCCT
TTTCCTGAATCTACTCTCAATGTTGACAGCACTGTGCGATTTCTGTATTTGCCAGGGGCTTCTCGTGGGA
GGTCGGCTCCAGCCCTCTCGAGAACCCTCCGGAGACCGTGGGGATGAGGCAGCGCTCAGCCAGAGAAGCC
CTGGAGCCCAGGACCAGCGCTTCAAGTGCACCGGGCGGGAGGCAGGGGCGCAGGGAGGGAGGCTCTGGAC
CGGCCGCCCCTGCTGACTATGGCCCCGGTGCAGGTCAAGGCTTGGGCCCGGGGATTTCCTTTCAAAGGTG
ACTTAGAGCCGCTTCATTAGGGTAGCGCCCTCCCAGGCGGGCCCTAATGGCGTGTTTGGCAGAGTCAGCG
AAAAGCAGCGCCCTGCGGCTCCAGCGGCGGGGGAGCGCCCTCTTCCGCGCCCTCGCCCTCTCCCGTGTGG
GGCAGCTGCGCGCACGGGGCCATTTGCAGCGCAAGCAGCCTAGCGTGCGGCCGCGGCGCCCAGGTCGGAA
AGGAGGCGGCATCATGGAGCTGAAGAAGGACATCAACGCTGTATCTATCGACATGCTGCTGATCGTGCAC
TCGGAGAAGCGGCGCGCCGCACAGGGTACGCTCTCAGACCAGCAGGCGAACCCGAGCTCGCTGCTGCAGC
GCGGAGGAGGTAACGCGCGCCCGACCCGGGTCCCCGGGTCCCCCGGCACCTTGGCCTCCTTGCCACAAGG
GCCTGCCCGCCGGGGGGCGCCCATTGCTCCCCCGAACGAGGTCAGCGAACCCGACCCTCTCCCCCATGGC
TGCCGTCGCTTCCCCGCAGTGGCACCGGCGTCCCGTGGCAACGCGCAGTCCCGTGGCAACGCGCAGCCCC
GTGATTGCCCCAGTTTCACTCCCTGTGCAGGCCTGTGGGTGCTTCCGTCACCCCTGGGAGTCTTTTTCCT
CCCAGCCACTCCCTAACGGGTGTAGAGCAAGATTGGAAGATACCCTGGAGGAAGGCGGCCAGAGCCTCAG
GGCTGACTCATGGGTAGCAGAGGCCTGCTCCCCCCCACAGCACACTGCAGGGCACAGGGGACGCTGGGAA
ACCTTTGGGCTGAGTCAGACTTTTAGATGGGGAGCTCCCTGTGTGGCCTTGCACAATTTACAGCCTCAGT
TTCTCCTTTCTTGAGACTTTCTCCCAGCCTCCTATAGATGGAAGGCATGAAAGGCGTAAAAGGAACGCAG
GTTTGGATAGGGTAAGATTTGAAACCTGCTGTCTCCACTTGAACTTATCCATCAATTTTACTTGTCCTAC
ACACTTTCTGGGTAAATTTGTAAGTTGTAGATTGGTAAAATAAAGTGACTGTTTATAGAGTGATTGTGGA
AAGCAGGATCCGGGCCTCTCTGTTCTCCAGACGACAGAACACAATGTGAATGGAGTACAGAGGGATGATA
ATGGAGGTTAAATAAGCCCCTGAAAAGCCAGAGAACCTCAGGGTCCTGGATGGCAAGCTGTCTCCGAGGC
TGGTGTGAGGATGCACTAGGCGGACTGCGGGCCAGTTCTTTAGGAGTTAGGCCTGACAGCCCATGCAGGA
GAATGACTTCTGCCTCTCTCCTCCCCTCCAGCTCTGGAGCTCCTTGGAATTCCTGCTGCTGCTCTGCCCT
TGGTTCTTCTGTCCCCAGAGCCTCTGTGCTCATTGAACATCCACCTTCCACCTGCCTTGCTTAAGTAATG
CTGCTGTGGATGGCCTTGCCACTACCAGCCACATAGGCTGGAGCTGCCTCCAGAGCCCTGGGAACCTCTT
GGGAAGGCAGGCAGGGGAAAGAAACAGAATCTAGAGGGCTGAGTATGTTGACAGGCGCTCTAGTCTCAGG
GCTGATTCCTGACTGAAGCAGGAGTTGTTTGGATTCGTTGGCTCTTTAGTTATCAGACCTACTCTTGGGA
GAAGGGGAAATGGCCTGCCCAGGGTATTTGTAGTTAGAAAGTCAACAGAGGGCATCAACTTCTTGATAGA
AATGAGACTGCTCTGAATACATGTACCACAGTCCAGAATGCCTTCTCCAGTGTGTGGCGAGGCCTAGTTC
CAGGTCAGCCCACGAGTCCCCTACTCCCAATCCTCTCTGATCACATCACTTCTAAAGGCTCTGACACTAG
AAAGGGCAAAGAATGCAGAGAAGCCCAGGAAACACCAGGCATCAGGACTGCCCTGAGAGGTGCCCAACAC
CTAGCACAAACTCGCCATGGCTCCAGACTCATGTATGCACACTAGTGACTATGACAGAGAAAAAAAGACC
CCTAATGGCTGGGCCCTTTCTGCTGAACTTTTTCTCCAAATATTTTTCTGATGGCCGTTTCTCACCGATG
CTATGAATGACAGTGTAGAGATCTGTGTCACTTATTCACAACCAGAGCAGAGAAGAGGAAAAGTGCATGG
CTTTTGATGCCTGGTGGGCAGAGATGATATTCAGGAGACCCCAGGCAAAGCCAGCCTGCAGACCCTTTCC
TCGAAAAACAGCCTAAGGGAGGAAAATGCCCAATAAGACCAGCTTGTTCCAAAGCCTCCCATGGGTGCAA
GATATAAAACAAACTCTTGCTCACTTCCAGGTATCTCCTCCTTTCTCCTCCACACTCCAGATCCTCCACT
GGAGAGTGGATCTTCCTTGGTTATATAAAAGAGGGCCTAGGCTCCATGTCAGAAAAGATCAAAGCTCAAT
TATTGTTGCAATGCAGAGGATGGAGTAATCCGTAACGAGTGGCCCTTCTGCTGGAAGGGCCTTCTGCTGG
AAGTGGCCCTTCTGCTGGAAGAAAAGAGTCAGGGGGACAAGAAGGCAAGGAGAGTGTGGACAGGCTAGGT
CACTAGGAATTTGGGCTGCAGAGCCAGCAGATGTTCTCTCCCATTTCTGTAAGGCCCTAGCAGCCTCCCA
GTCATTTCCCTAGAACAGTGCTTCTCCAAAACGTGCCTGAAAGCCCTCAACTCCTATCAGAAAGTAGAGT
GCATATACTCCTGCAGCAGTAGAGGATATAGCCTATAGCTGCCTGGGGCAAGACCCAATGAGCTTTGAAG
TCTTAGGTCTTATCCCAAAAGTCTATGTGGTTTGTGTATTCTACTCCATAGGTGTGTCATGAGGGTTCCA
AGTGGTAAGTCACTGGCACTGGCTATGGGGGGCTCTGCAGAAAAGAAGGGTGTTAAGAAGAGAGTGGCGG
CCCCTGAGTGGTTGAGAGGGCTGGAGAATAAGACTCAAGGCTGAGCTTCCAGGAACAGTACCGTAAGCAG
CACCAACGAGCTGGTCTAGTGAGGAAACCTCCTCGGCCACCAAGGAGCACTGAATACTCCTTCCGCGCCA
GGATCTCAATTTTATTGCAGCAGCTCCCGCTGCCAGAACGTATCCCCATGCTGTGCCAGGAACTCGACCT
TGCGGCTACCACTTGCAGCCGCAGCCAGAGCGAGAGCTTTCCTTCCCTTCCCTGTTGCATCACCAGGTCC
CAAGGCAGAGTGAAACACAGGTACATCAGATTGGCCATGGCTAGGCCATACGTGTTTACCCTCTAGCTGC
AAGGGAGGCCAAGAGAACTGGCAACTGTCATTTTCCATTCCCATTGGGATTGACGGTCTTAGCCCACTCC
CCCCACTCTATATCAAGACTCTAAAAGTGGGTGATTCACCACATACAGAAATAGGCAGGCAATGTGGATA
GCAAATGACTTCTAAAATAATGCTTTCATATGTATGCAGTGGAGAGGTGGAAGTGGAAAATGGGCATCTG
TACTACAAAAATTCTCCCTAAATGACCCTGATAACCAACCCTGCTGCCCTTGAAAACTGTTTTAACAGTT
TTTAAATGTTTTGAATTTTGAGAGTTGCATTCATTCCCTCAAGGGCATCTTATTCATGGGTAGCCTCTCT
CAGATTCCAAGAGAGCCAAGATCCACCTGCTCTACCATAACCTAGATAAGTCTGCAACTGGTACATCACT
CTTTTGCCCTGAGTCTCCCACCTCAGAGAAGCTTGTTATTTTGCAGGATTCCAAGGTGTTGGAAATGGAG
TCCGAAGATGGCAGAAATTAGAAGGAAATGACTTTCACGGTAAATTCTCTTTTTCCTTTCAACCTATAGG
ATTTAATACATTGAATAATAACTGGAAGAATTGGGTTTCACTCATTTGAAGACTAGCCCTAGACATCTAA
CCACTGAACGGAAAGTGGCTGCTCCTGACCCAGCCAGGCCTCTAAGGGCCACTGTGACTGTGGGAAGTGT
TCCCTGGAGTCAGGTCCCCTGCTCTCCTGGAGGCTGTGGCTGAAGAGCAGTGCCAGGAACTTGAGCACCC
ATTTGTTTGCTGGCTGAGCCGTAGGGGAGGACTTAACATCCCTTGGGATGGTGAGGTTTGAGTGTGCTCC
TATGAGAGGCAGCCATCTATCTTTTGAAAATGTTTCCCAACAGAGAGGATAGTCGTCTGCCTTGGGACAG
TATGACTTGAAGCCTGAGAAGATGCCACTCAAGGGTTCTATAGCTCCCTGCTGGCCTGGGACCCAAAATA
CAATAAGAAGTCAGCTTCCAAAGCCTCTTTTTTCCAGTGCCAGGCATCTGCCACCCCAGGCCCAGGATCT
CTGTGGTAACACTAAGGGGCCAAGTATAATGGGCTTTATTCTATTCCCTTTAGGGTTCATTAGCCCAGCC
TCCAGGGACAAACATCAGGGACACTGCACAGTGGGCACGTGAAGGTTCTAATAGAGACCAAACCTCTACT
CCCAGAGCCCCAGTAGAGAACTCCATCAGCCCGCAGCTATGTGAGCCCTAGGATCAGATGACTGGCTTAT
CAAATGCCAGTGTAAAGGAACCACCCTCCATGACACAAATGGGGAAATAGGATTGCAGATGAATAGGACT
AAACTGCTATGTTCTACCATCCTTCTGACAATAATGAAAGGGAAAGATGAAGTTCTAAAAGTGAATTCAT
TCACTCATTCTGTTCACAAATGCCTATAGTGCTTACTTTGTGCCAGGCATGATTCTAAATATTGGAAATG
AAACAGGGAAAAAGACAGCCAAGAACACTGCCCTCAGGGGAGCTACATTAAAATGATTGGAATTATAATG
CACTGCAGGGGAGTCTCAACACCCTGGAGTTGAAGAGTCTTCCTGTGTCCCCTGCCTTTGGTAAGAGTCC
TCAAGACACACTCCCCATCCCCACCCCACCACTGCCAGGGAGCCACCCTGACAGGCATGCATGGGTGCAA
TGGCTTCCCTGTAATCTTGCTACAGGGGCCATCAGCTGGTAGACATGCCAGCTTTAGCTCTACTGAAGTA
GACGCCAAAGCTAACAAAACTCTGCTATGGGTTATTGAGAATTTTAATAAGAATCAGACCCCCAGAGACC
TGTTAGCCACAGAAATGGCCATGCAGGGCATGGAAGGTATGCATGCTAAGCCCATGTGTGAGCTGGTGAG
TGGGCAAGCAAGCCACAGGTGGAAACAAAGAAGCACAGCATCCTTGTTGGTTGCAGCTGTCTCATACCTT
GCCTGAGTTGTCTGACACTCATCTTAGGGCTTCTCAGGCATGTTATTACATCAGGAGAGGATTGAAATAC
ACTGAATTCATTCTATTTTTACCATCTTCCGGGTTTCTGATTTGGGAGAAGTTTAGTGGAAAAATATCAC
AAAACACAAAGTGACACATTGACTATTACAAGACCATTATAAGACTCTTGTAAAAGACATGAAGTGGGTC
TTAGCACTATGTGTCAGCTGATAGAAGATGACTAAAATAGTTCTTGCAAAACCACCATGAGAACTGGAAG
TTTCAGGTAAATGCAGGGCTGGACAGAGGCTCCACAGAGGCCCTGAGCCCAGAAAAAATGACGCTTCTAC
CACTCCATCAACATGTGTAAAGCGAAATTTCATTTATTTTTCAAATGGTACGTTTGCATGCATGCAGAAA
TTAAAATTAGTTTCTTTCTGTGTTTTCTGATGTCGAGATTCTGAGTGCAGGTATTGAAGCTGACCTTTTC
TCATACCTGCCTTACTGGTGCCCAAGGCACCCAGCCCTGGTAACCAGCAGGCCGCCCAGCCCTGGTAACT
GGATGGCTTCCATCTTAGAAGGAAGTGGCTGATGGCATGTGCTGGTGGAGTGCACACAGATGCTGCTGAG
CATGGGGTGGATCCAGAAGGGCACATTTTCCCCCTCTTGCTACCCCGTGCACTGCTTGGGCTGGAACACT
GGCAGGGTGTTGAGACTAGAGAGCTCCAAACTTTTCGAAGCAGCAGGTGTTTGGGCCCAGCCGCCTCCTC
ACCCTCCTACCCCACCCCAATAGCTTGCCCAGCAACGGGCTTCCCCTGCTGTGATTGGCTACTTTCCTTG
GTCCTGGACAAAGTCTGCCTAGTAGCACAGGGACAGACGCCCCACAGCTAAAGGGAAACCAATTGTGCAG
CCTTGACTATAAATACCCCACAGCAGCTATGCCAGCCAATCACCCTCCTTGAGGATTCCCTTCCCGGATC
TTGTTTCCTCCCTCAGCCAGCCGAGGCACTGCCAGCTGTGCCTGCAGAAGGCGGTCCTGGCCTCCCTTTC
CTCGCCTGATCTGGGGCTCCAGCCCCGCATGCCACATCACCCTGTATTGGAATACACAGTGGACCTAGCC
CTTGGCTGACTAGTGTGGGGCTCAGGGACCTAGCTGGGCCTGTTCATAGGAGAAGGCTGGAGGAGGCAGG
CTTTCCAGGGCAGGACTCTGTCAGGTGGATCACAAATCCAGCCTGGCTCAGTACCTCACCCCCCACCTTG
ATTGGCTTCTGCATGGGCCCAGGCACGGGAACCACTGAGCGGCTGATCTGCACAAGAAGAATCATCTAAA
TGCACCTGTTCAGGCCAATGCCCCAGAGGCATACTTGTTTCCAGACTAAGGCACTGGTCCCATTGGTCAC
TTCCAGTGACAAGCCCACTGCAAGCCAGCAGGAAGCTATAGAGAGAAGATGGCAGGCCAGAGTGGTAGTG
GTGGGGGAACCTCCAGCTGCCACCATCCTCCCCTTGCCCAGCCTTCCCCCCACCCCTCTGAAGCAGAGGT
TCAAGGGCAGCTTCAGTAACAGCAGGCCACAGCAGCAGCTGTAAAGAAGGGAGGAAAGGTCATGCTTCCC
CTGGGAGGGCAAGTAAGCCTGTGAGCTCCCGCTTCTTGGCCTATGGCAGGAACTAGGCATCACTCCATGG
CAGCCTGACCCCACTAGGCTCTGAGGACAACTCTGTGTCCCAGGGACAGAAATATCCCTGCATGTGGAGA
GAACGTGTGTAGCGTGCATGCCCTGCAAATGTTTGTGAAACAGAACCAAACTACACATCATCCCGTCTCC
TAGGCTGGGGTAGGAGGCAGGCTGGCCCCCTGTTCTTCTCATTAAGAAGCAGGAGGCTCGGCCGGGCGCG
GTGGCTCACGCCTATAATCCCAGCACTTTGGGAGGCTGAGGCGGGTGAATCACAAGGTCAGGAGATCGAG
ACCATCCTGGCTAACATGGTGAAACCCCGTCTCTACTAAAAATACAAAAAATTAGCCAGTGTGGTGGCGG
GCACCTGTAGTCCCAGCTGCTCCGGAGGTGGAGGCAGGAGAATGGCACGAACCCAGGAGGCGGAGCTTGC
AGTGAGCAGAGATCGCGCCACTGCACTTCACCCTGGGTGACAGAGCGAGACTCCGTCTCAAAAAAAAAAA
AAAAGAAGCAGGAGACTCAACTGGGAGTCAGAAGACCTGAGGTTCAAGTCCCAGGTCAGAACCTCCCAGC
CTGCTCCACAGAAATGTGTGAAGATCAATGAGGCATCTGTGTATGCAGTGCTTTTGAAACTGTGCCTGGT
GACACCAAAGTTGGGAGTTATGGCTACAAAGACCATGGAAGACTGGTAGCCTGTTCCAGAAGAAACCCAG
CCTGTGCTGGCACCTGCTAAGAGGCACAAGGGGTGTGTGCACAGGGGCTTCCAGGCATTTGTCTCCATGC
ACAGTGTCGTGCTGGTTTGCACATGTGCACACTGTGAGGGTGTGCACACATTTAGGCCTGGGGATGTGCT
TGGCCCCCGCCAGCCCACCTTTCCCGCAAACACATACAGCTCTTTGCTCTGCCTGGGGAAAATTCCCCAG
TGGTCACTGCAGGCCTCTCTGTCTTCCTTAAATAGAAAACTTGGTGGAGAAGCAGCACCCTCAGCAGCCC
CAGGTCATCACTTCCTACAACAGCCAAGGTAAGTGATTCGCCGCTAGCCGTGACCTCCCCTGCAGAGGTG
TGCCCAGACCTGATGCTCTGGCCAGCGACAGCCCAGGATGTGGAGCTCAGACTATGGGGAGCAGGGTCCT
CTCTGCTCCCATGGGTGTTGGAAAACCCTTTTCCTCTCCCTGCGTCTCATGGGAAGCAGCAAGAGGCTGC
ACAGTCTCAGTCTTCCCCTCACCATGAGATCAAGAAATAAAGCCCATGGGCCTCAGAGCACGCTGCTGTC
ATCATCATCACTATTACTGTCATCACTATTCAACATTCAACATTTGTTGAATGCTAACTATGTGCCAGAC
ACCCTGCCAAGCCACTTATGCACTTATTCTCATGTAAATTAAGCTTCACAGCAACCCTGTTTGACAGGTA
CTCCTCTTTACCATCCTCAAATTACAGATGAGTAAACTGAGGCTTGGAAAGGCTGTCATGTGTTCAAGAT
CACATGGCCAGCAAGAGTCAGAGCTGGGCAGTCTGAGCCCCAGCAAGGCCTCAGCTGTTAACCTTCACAC
ATCTCTGCTTCCTCATCTCCCCTGGAAACCAGCCTCCCTCCCTCTGGCTCATGGCATCCAAGCCCTCCCT
TGTCCTGACCAATAGCAGTTCTTCGGCCTCTCCTGCTGAGACTGAGCCTTCCCAGGGCATCCCACCCAGT
GAGTTTGCCACAGCAAGGCAGAGTGAGAGGCACCTTCATTGGGAGTCCTCACTTTTTGGGGACAGGCTCT
GGGGACTCGGGGAAGGCAGGCAAGGGCATCACTGACTACAGTTGACTGGATCTGAGCACACGACAAGATA
GAGACAGGAGGGCCCCACAGCAATGGTGACAGGAGGCCAGGTTAGAGACTCTCACGCCCACCTTGGTTAA
CTGCTGTGCATTAAATACTGGGCCCTGGTGTCTGAGTATTCTCACAGGCTCAGCCATAAGAGTTCATCAC
GCTTGCTCTGAGAGTTGATGTCCTAACCTATAAGGAAATTATCTGATCAAATTCAGAGGCAGTGATTTCC
CCCATGCCCCATATGAAACAGGAGGACTCTGGAGGCCCCGGAGGATTGGAGCCTCTCAGGGAATTGCTCT
CTGAAATACAGGTGGACAAGCTTCTCTATAGAAAGGCGCTGGCAGGTAGTGATGAAAGGCCAGCCAACTG
GGATTACCAGCATGGGGGAGCTGGCTCTCCTCACAGTACAGGCCAGTGTCCCTTTAAGCCAGAATTCCTG
AGAATGCAGCTCTATGACAGCCTTAGAGACCTCTGTGGGCCCAGCAGGACCTGCCACCAGCCCTGAGAAC
TACAAGGACAAGCCCATGCTCTGTCCTGTTGCTTCTCTAAAATGAACTCAACTGCACATGGCTCAACACA
AAAAAAAGGACTCAGAGATATCTGGCTATAATGAGAGTGGATTAGCACCACTTACCTCACTTAGTGAAGA
AGACAGACAAAGGCCCCTGTCCTTGTGGAGCTGACATCCTAGTGAGGAGACACAGACAATCCTGACAAGT
CAAACACCCTGAGCCAGATAAGGTATAAGGGGGAAGTAAAGCAGGAAGGATGATAAGGGGTGTGTGCGGG
TATGGGGTCTGGGGCTGTGCGGGGTTGTATATTCATTTCCTATAGCAGCTACAGGAAATTTCCACAAATG
AGGTGGCTTAAAATACTGCAGATTTATTCTCTTACACTTCTGGAGCCCAGAAGTCCAAAATCTGTTTCAC
CGGGCTAAAGTCAAAGTGTCAGCCGGGCCAGCGTTTTTTAGAGGCTCTAGGGGTGCATTCATTCCTTGCC
TCTTCCTGCTGCTAGAGGCTGCAGGCCCACCATGGCTTGTGGCTGCCTCACTCCAAACTCTGCTTCCATT
GTCACATTGCCTTCTCTGCTTATAAATAGCTGACTCTCCCTGTGCTCCCTTGTTTAAGGATATGTGTGGT
CGCATTCAGAGCCCAGCCAGATAATCAGGATGGTCTCCCTATCTTGAGATCCTTAATTTAATCACATCCA
CATAGTCCTCTTTACCATATTAGGTAACATTCACAGGTTCCAGGGATTCTGGCATGGATATCTCTAAAGG
CCATTCTTCAGCCTATCCCAGGGGACAATTTAAAGTGGAGTAGGTGAGAAAGGCCTCACTGAGAAAGTGA
CATCTAAGCAAGGAAAGAGGTAAGAGCAAATGATGTGGGTATCTGGGGAAGAGCTTCCAGCAGAGGGAGC
AGTGAGCGTGAAGGCCCCCAGGCAGGAGCGTGTCTGCATGTTCAAGGAGCAGGAAATTGTGCTGTGGAGA
GGAAGAGAGGGGCATGGGCAAGGTGGGGGTAGCAGAAGAGCCAGAGAGGACACAGGGCAGAGTTGGCAGG
GCCTGGGAGGCTTCTAATCTGAGTGTGATTGGAAACCATTAGATGGTTGTAGACATCCAATGCGTAGAGA
ATAAACAGAGGGGGCAAGGTTGGAAGCAAGACAACCCTTAAGAGGCATTGTGATAATCCAGCAAGAGAGG
TTCCCCCATGGTGTGGATTTGGGGTATACAAGAGAGGAGTCAAGGCTTGGGACCTGAGCAATGGGGAGGA
TGAAGATGAGGGAGATGCAGGAAGAGCAGGTTGGGAAAGAAACATGAATTCAAATTTGGCTTTGTTGAGG
TTGGTGCCAGCACCTGCCATTTTGGAAAGCTGTGGCCAGGGAATCGTTACTCCATGTGCCACTTTCTTAT
TCATCTCATGAGCCTTGACAACCTTTGGGCTCTGTTCTGCGAACCACACCGGCCCCCAGAGAAGCCTTCT
TGGAAGCTCTATCACAGTAATAGCGTTGACCAGAGGTGGGACACTTGGTGTGTGCCAGATATTAAGCTAA
GCCCCTCTCACGGCTCACCTCATTTAATCTCCACAACCACCTTTTGTGGAAGGTTATCATCCTCATTTTA
CAGATAGGGAAGCTCAAAGATATTTAAATAACTTGCCTGAAGTCGCATACCAGACCCAGAAGTTGAACCC
ATGTCTGTGGGCTCTCGAAAGCGGTGGGCTTAACTACTCCCCCATACTGTCTCTTCCATACAGGACAGGG
ACCAATCTGGTGGTGTTGATTTGGGTGCAGTACCCCCCACCCAACCCCCACCCTCAATGTGCAGTCTGAG
ATTTCAGTCTGAACTCATTGCTCGGAATGGTTCAACATTTTCTCTCCTCCTCAGCCTCTCTTTCAATGGT
AGACTTTATGCAACAGGTGGCAAAGGAACATTGGAATCTTGGGTCATGGGAAGGGGCTTTGGAGATGGAT
CTGTAGTGGGTGTGTGGGTTGAATGAATTTGTAAGGAGAGTAATCACAAGGAGCCCCTGCAAAGAACATT
CTTTCTTTTTTTTTTTTTTTTTTTTTTTTTTTTTGAGACAGGGTGTCACTCTGTGGCCCAGGTTGGAGTG
CAGAGGTGCGATCATAGCTCACTCCAGCCTCAAACTCCTGGGCTCAAGCAATCCTCCTTCCTCAGCCTCC
CGAGTAGCTGGGACCACAGGTGAGTGCCACCACATGGCTGGTTTTTTTTTTTTCGTTTTTTGTAGAGATG
GGGTCTCACTATGTTGCTCAGAATGGTCTCAAACCCCTGGGCTCAAGCAATCCCCCTGTCTCAGTTTCCC
AAAGTGCTGGGATTACAGGCATAAGCCACCATGCCTGACCCCAAAGGACATTCTTGAGGAGTCAGTTTTC
TGTTGAGGCTTCTGTCCTCAAGGTCATCAGCCCTTATCCCCATGCAAACACTTTGTAGAGTAGTCTTCTT
TGATGTGTGCACTAGGGTCAAACTGCCATGTTTCCAGACCTCAGACCTCCCTGTGGAGCTCCAACCCATA
GAAGGAATCATGAATGGTCCTTTGTTTTCCGTTCCTAATGGAAGCCATGGGCAGGGCTGCCAGTGATGCC
CTTCATCATGCACACCAGCAGATGCCCAGTCACAGCCCTGAGCTCTGGAACACTAGGTTGTCATCTCATA
ATGAGAGCTGCTGAGTGGGGTTTTCATGCTGTGTTTGGCAGTACCCCCTAAGGGTGGTGTTCCATCATGT
TCTACAGAGCTAGACAGTCCCTGTCACCATCTAGCAGGCTCATTCCTGGGATGTCACTGCTTTCCACTTC
CTGACTACTGCATCCTGGGTGGAATCACAGCATGTCTCTGTTGAATATCTAACCAGGGACTCTTTAAGGG
TATTTTTTTTTAAACTACCAATGCTTTAGCTGTTTTCCTGACTCTAAGCTACTGCAGGAAAAAAAAAAAA
AAACTCCAAAGTAGAAAAAAAAAAAAGGCTGTAAATGTGCCAACCTGTTATAGAAAAAGTTAACAAACAC
CTTAAACCAATGATGGCAAGTATTTCACATGAATCCCATGACCCCTCCACACACATATACACCCGTGTGC
ATGTGCACATGCACACACACACACACCTGACAGTCTTCGTTAATTGATCCCTTCAGGCAGCCACTTCCAA
TCAGTCAGAGTTAATGAAAAACATGAAACCTGTTTGCCATACTGCGTTAGGTGTTCTGAGCCAATTAGGC
CTTTAGATAAATCCTGATTGAAACCTGAAGAGATCTGGAGAGCAGCTGTCAGGTGGAAGAAACCTGACTG
GGTGAAAGGCAGGGCAGTAAGCATGTGGACATTCTTGTGCTTGCCTGTATGCTCTGTTTCTGAGAAGGCA
GGGAAAAGCAAGGAGAGCTGGCGGGGTCTAGGAAAGGGGCCAGGGAGCCCGTTCTCAAGATTAGCAGAAA
ATAATCTCTAACATGCTCTTAACTAAGACGGTCAAAGCTGCAGCTTCAGAAATGTGATTTTGTTGACAAC
TGGTGGAGCTGGGGAGGGATGCATGAGAGAGGCCAGCCAGGCAGCCTGGAATCCCAGGAAAGGCCTCCTG
TAGGTTTGGTTGTGGTGGTTAATAGTCAAGATTGGAATAATGTACTGTACTTAGGCTGTACTTAGGGTTT
AGATCTGACTGCGAATTTTACTCCAAATGATCTACTCTCTGTTAGAGATTGGCCACACTAATATTTTTTC
TTAAATTAAGAGTTTTATTTGATCCAGTAGCGTCATAGCCAGCAAACTCTCTCCACTCCATGCCTTAAGG
AAGCATAAAGCTATATTGTGAATGCTTTTCCCCTTGAAACAGCGTGTCCAGCCCAGGCCCAGTTCCTGGA
GCCAGCAAAGCCTCCCAGTGGTGCCCTCACACAACAGTAAGTGAGAGCAGGAATCACACTCCCATGCAGA
TCAGGGAGGACGAGAAAGTGGCTGTTTTCCTTGAGGGAATGCTAAGCAGCAGCACAGCTTTGGGGGAGAA
CCGGGAAGTGGGATTCAGGAAAATATTGGACATTGACCCCTGTCCAAGAGGGAAGAGGAATCACAGAAAG
GAGAGAAGGGATTAGGAATAGTGATGGCTGGAGCATCTGGGCCAGGGCCGTGCTCCGAACATGAGCCCAC
CTCAACACCCTGCCCTGGAGGGGCTTTAACAGCAGTTTGTTTTTCACCTTGATCACTCTTTCACAAAGAG
ACATGGCTGAAGTAGGACATGGCTAATACCCATAGAGACATGGCTCAGGGCATGTAACGCAGGATGGCAA
ACAAGTTTCATGTTTTTCATTAACTGTGACTTATTGGAAGTGGCTGCCTGAAGCGATAAATTAACAAAGA
CTGTCAGATGTGCGTGTGAAAGTACCTGGGCATTATGAGAAGTTTGGGGAAGAGATTTGTTGTTGAACCC
TATGCCAATAAGTTGTTCACTTTTGCAGTTTCTCTAAGCTTATGGAGATTGTTCCAGTCCAGCATTTTCC
AAATGGTGTTTGACAAAATAGTACTGGGTCAAATAGAGTTCTTTACTACAGGACTACTCAGATCATTGTG
TATTGTGACCTATGAGAGGGGAGAGAGTGAACATGTTTCTCAATTCATTTTTCACAAGGCATCTTGAAGT
CAAGTACCACTTAGCACTCACTTTAAAAGACACCTCAGAGGGTTTGAGGTGGAGAAAGTGAAAAGGAAAA
GAACAGGCCCATGCCTCACTGATGATGGGAAGACACACAGAAAGTATTTGCTGCTGATGGCGATAGCACC
CAGTCCTACGACCAGAACTGAATTATGCCTTGAACTCTGAAATGCTTAATGTTTTAATTACAAACTCACA
AGGCTGATTTTCTCTCAACATTGAATATGCTGGAAACTACACTTCAAAATGAATGTTAACCCCTTCAAAG
CTATGCTTTGCAGAGGCCACATTGTATCTTTTCAAATTGTTTGAGAATGACCCTCTTTGGCATGCAGAAC
AAGTTTATGAGCTACACAAAAAAGACAGTATTGTTATTCTATGAAAAATAATTGCAATGCATTTAGCAGG
CAAAGGGTTAGTAAAAGGAGGTTTTACAAAATCAATGAGGAAAAAATAATACTTCAATAGGAAAATAGCC
AATTCACAAAATAAGAATTTAAAATGGTAACTAAGTATAGGCAAAGACTACTACAATAAAACTAAAGAAA
TATACAACACAACCAGGTATTATTATTCACTGATAAAAATTTAAAGGGTTGATAAAACCAAACATCAGTG
AGTGAATATGTGATCTATTTTTGCATTACAAACCACCACACAACTTAGCAGCTTTAGACAATAAGCATTT
ACGAGCTCACCAATCTGCAGGTTAGCTAGAAATTCTCATTGGAGCCACACCCAGCTGATCTTGGGTCTTG
GGTGGGCTTGCTCATGTGCTTGTTGTCAGCTGGTGGGTTGGCTGGAGACCAGCTGGTCTGGGATAGCCTC
ACCTGAGAGGCTATGCTGTGCTCCATGTGGGCTCTCATCTACAACCTGGCTCATTCACATGGCTGTTATG
GCTGGGATCCCAAGAATGAGAATGGAAGCCTCAAAGTTTCTAGAAGCCTAGGCTCAGAATTTGTGCAACA
TCACTTCTGGCATGTGCTAATTGGCCAAACCAAGTCAAAAGCCCAGCCCAAATTCAAGGGGTGGAACAAA
AGACCTCTCATTGGGAGGAATTGCACTTTGTGACCAGTTTTGCAATCAAGCACAGCAAGAATGTGCACAA
TGGGCATTCTCTTGCACTACTGATAAGATCATAGATTGATAAAATCATTCATAAGAAAATTTTGCCAATA
TCTAACAAAAGCCTTTAAAATATCCATATTATTTGATTCATACTTCAGAATTTATTCAAAGGAACTGATT
TGCAAAGTGTGCAAATCACTTTACATACATACAAAGATGTATGTATCTGAGTGTTCATCACATCATTTCT
AATAGCAAAACTGTAAAAAGCTAAATATTCAACAAAAAGGGATTGCTTATATGGATTATAAACCATCTAC
AAAATAGAATACTATGTAGCCATTTGAAATGATAACATATGGCTATGCTCACTGACATGTAAAGATTTAA
ATGAAGAAAGCAGGTTATGAAAAATATGTTTACTATAATCGGCTTTGGATTTTTAAATGTGGAAATGAAG
TCTTAAAGGAGTTTCACTTTCTGCTTTATATCGTTCTGTATCATCCAAGTTTTTGTTTTAATTCTGCTTG
GGTTTTTTTGTGTGTGGTTGGTATTTTTGCTTTTATTTTTATTTTAAATTAACATACAGTAAAATCGAAT
GTTTGTAGTGTGTAGTTCTGATTGCTAACACATGCATAGATTTGTGAAACCACCACTATGGTGGTTACCA
TCATCAGAATATCCAGAATACACCACCATCGGAATATACAGCAGTTCTATTTCCCTCTTTAAAATTATCT
TGTGTGGACTCTAATCCTCCCATCACTCTTAAGAGTTCCTTTGTATTGTTGAGAAGTACTTTTTTTTTTT
TTTTTTTTTTACAGAGTCTCGCTCTGTTGTCCAGGCTGGACTGCAGTGGCACGATCTCGGCTCACTGCAA
CCTCCGCCTCCCAGGTCCAAGCGATTCTCCTGCCTCAGCCTCCCGAGTAGCTGGGGTTACAGGTGCCTGC
CACCATGCCCAGCTAATCTTTGTATTTTTAGTAGAGATGGGGTTTCACCATATTGGCCAGGCTTGTCTCA
AACTCCTGACCTCAAGTGATCCACCCACCTCGGCCTCCCAAAGTGCTGGGATTACAGGCATGAGCCATCA
TGCCCAGCTGAGAACTATTTCTTGATATGGATGTACCACAGGTTGTTTATCCATTCACTCATTGAAGGAC
GTTTAAGTTGTTTCCAGTTCAGGGTGATTATGAATAGAGCTGCTATACAAACATTTGTGTACAGGTTTTT
GTGTGAACATAAGTTTTCATTTCTCTGGGATAAATGCCCAACAGGGCAATTTTTAGGTTGTATGGTAAGT
ACATGTTTTGTTTTATAAGAAACTGCCAAACTATTTTCCAGAGTGACTGTTCTATTTTACAATCCCACTA
GCAATATATGAGATCCAATTTCTCTGCATCCTTGTCAGCATATGATATTATCACTATTTTTAAATTACAG
TCACCCCTCAGTATACATGGGGGATTGGTTCCAGGACCACCCACATATAACCAAATGTGGGCATACTCAA
GTCCCACAGTCGTCCCTGCAGAAACCCCATGTAGGAAAAGTTGGCCCTCTCTATGCGAGTTTTGCATTCC
ATGAACAGTACTCTCCATCTGCATTTGGTAGAAAAAAAGGACCATATGAGTGGACTCGTGCAGTTCAAAC
CCATGTTGTTCAAGGGCCAGTTGCATATTGTTGCTCTTCTAATAGATGTGATGTCTCATTTTGGTTTTAA
CTTGCCTTTTCCTAATGGCTGATGATGCTGAACATCTTTTCATGTGCTTATTTTCCATCCAAAATCTCTT
TTATAGTAAAATATCTATTCTTTTGCCCTTTTTCCAACTGAATTGTCTTTGCACTTTTGTAAAAATTTAT
TTTGCTATGTTTGTGTGAGTCTATTTTTTATTATTCTGTTTATTGATCTGTTTATCTATCCTCTTACCAA
CATCACATTGTTGTGATTACAGCTTTTCATTAATGCATCTTAAAATCAAAGAGTGGGAGTCCTCCCATCT
TTTTTTTTCAAAATTAGTTTAGTTATTCCAGTTCCTGTGCCTTGATGTCTATATTTTATAATTATATATT
TGCACAATTCCCCCAAAATGTTTGGTTGAACTTGCATTAAATCTGTAGATCAATTTTGAGAGAATTGCCC
ACTTGACAATTTTAAGTCTTCCAATCCATAAACATGGTATGTCTCCCCATCTATTTAAGTCATCTTTAAT
GTGTGTAATCATATTTTGGAGTTTTCAGTATGCGATTATTGCACATGTTTGGGATTTTTGTTTGTTTTAG
ATTTATAACTAACATTTTAATTTTGGAACTATGGGAAATGGTTATTTTTAAATGCCATCTCCAATTGCTT
ATGGCCAATATACAGAAATACAATCGATGTTTGTATGTTAACCTTGTATACCGTGACCATGCTAAACTAA
CTTATTAGTTCTAGGAGCTTTTTCTTGTAGATTACTTGGGATTTCCTATGTAGACAGACATATCAACTGT
GAATAAAGACAGTTTTATTTCTTCCTTGCCAATCTGTATGCCTTTTATTTCTCTTTCTTGTCTTATTACA
CAGGGTGGGACTTCCAGTACAATATTATTAGGATCAGTGAGAGTGGATATCTATGACTTGTTCCCAATAT
TAGGAAGAAAGCATAACCATGTTTCACCATTAAATATGCTGTTAGCTGTATGCTTTTTGTAGGTTTCCTT
TTTCAGATTAAGAAAGTTCCCTTTTATTCCTAGTTTGTTGAGAGGTTTTATCATGAACGGATGTTAAATT
TTGTCAAATGCATTTTTCTACACCAATAGGTGTGATCCTAGTAGTTTGTCTTCTTTAGCCTGTTAATATG
GTGAATTACATTCATTGATTTCCAAATATTTAACCAGCTTTGCATTCCCAAAATAAACCACCCATGGTTG
TAGTATATTTTTCTTTTACTATATTGTTGGATTTAATGTTGTAATATTTTGTGGAAGATTTTTGTGTTTA
CATTTATAAGGGGTATTGGTCTGTAGTGTTCTTTTCTTTTGCTGTCTTTGTGTTGATATCAGATTAATGC
TAGCCTCATAAAATGAGTTATGAAGCATTCACTTCTCTTCTGTTTTACGAAAGATCCTATGAAGAATTGA
TGTTATTTACTTTAAATGTTTGTTAGAATTTACCACTGAAGCTATCTGGGCCTGGCTTTTGGGGGGAAGA
TTTTAAACTACAAGTTTAATTTAACAAATAGGTATTGGGAATACTTAGGTTATCTGCTTCCTATATGAGT
TTTGTGGTAGTTAGTGACTTTTAAGGAATTGGCCCATTTCATCTAAGTTGTAGAATTTATGTGCACAGAA
GTTTTGTAGCATTCCTTTATTATTATTTTAATACATATGAGGGTCTGTAGCGATAGCCCTCATTTCATTC
CTGATATTGGTGTATTTACCCCTGATTTGTGTCTTGCCTCTTTTTTTTCTTGGTCATTCTAGCTAGAAGT
TCATCAATTTTACTGATCTTTAAAAAGAACCAGCATTTAGTTTCATTGATTATTCTCTGTTGCTGTTTTT
AACTTCAGTGATTTATTCTCTTATTTTTATGATTTTCTTCCTTCTGCTTGCCTTGGATTTAGTCTTCATT
TCTCTACTTTCTTAAAATGGAAGCTTAGATTGTTGATTTGAAACCTTTCTTTTTTCTGTAATAAGAATTT
AATGCTACAAATTTCCCCTTGAAGCACTGCTTTGGCTTCATCTCACACATTTTGATATGTTTTATTTTCA
TTTTCAATGAGTTCAAAATATTTTCTAATTTTCCTTGAGACTTCTTCTTTGATCTATGTGTGATTTGAAA
GTATGTTAATTTTTAATACTTGGACATTTTCCAGTTGCTTTCTGTTTTTTACTTCTAGTTTAGTTCATTT
TGGTCAAAGAGGAGTTTTGTATGATTTCAGTTCTCTTCAACTTTTTAGGTTTGTTTTGTGACCAAGGATA
TAGTCCATCTTGGTGATTATTCAATGGGCATTTGACATGAATGTGCGTTGTGCTGTTATTAGATGGAATC
TTTTATAAACATCAAATAAATTTTATTCATTGTGTAGTTCATTTCTTCTATATATTTGCTGATTTTCTTT
CAATTAATTCTCTCAATGACTGAAAGATTAGTGTTAAAATCTCGAACTATAATTGTAGATTTGTCTATTC
CTCCTTTTAGTCCTATCAGTTCTTGTTTCATGTATTTTAAAACTTTGTTGTTTGACGTATGCCTATTGAA
GGTTGTTATGTCTTCTTGCTAATATTCCTCTATCCCTGATATTTTCCTTTCTCTGATCTGATATTAACAT
AGCAACTCCAGCTTTCTGTTATCTTTTCACTTAACCAACCTATATCATAGTAGTTAAAGTTGGATCCTTG
GAAAGTGAATATAGTTTAATCTTATGTATTCACCCTTTCTGAAAATCTCTATTTTTCAATTAGTGTGTTT
AGACCATTTACACTTAATGTAATTACTGATACATTTGTATTTAGGTTTTCTACTTTATTATTTGTTTTCT
GTTTGTGCCTCTGTTTCCCCTTTCCTGCTTTCCTTGCCTTTCAGAATTACATTTAAATTTGCCTATTTTT
TTACTATATCTCCATAATAGCTATTTTTGTATGTATTATATGTACTTAACTTTTCATAATCTATTACAAT
GAATATTAACATTTCAAGTAGAATGAGGAAGTCTTACCTCCATATAGGTAGATTCACTCTCCTACTCTAT
GTTGTAGTTGTCATATATTTTACATCTAATATATCAAAAACCTATCAGATGTTATAATTATTTCTTTCAA
TTATTATTCATATTATCAAGAACTTGAGAGGAGGGCAGACTAATATATTTATCTAGATATTTACCATTTC
TCTCCCTCTTCCAAGTTTTCCTCTGGTATAATTTCTCTTCCGTCTGAAGAACTTCCGTTAGCATTTCTTT
TGGAGCAAGTCTGTTGGTACCAAATTCTCTTGGCTTTACATCGCTTTTCACTGGCTACTTTCCATATTTT
TTTCTTTAATAGTTTGGTTATGTGTCTGGACTTGGATTTTCTTACATTTATCCTGTTTAGAATTCACTGA
GTTTCTTGAATCAGTAAGGTTTTATGTCTTTTGCCAGATTTCAGAAGTTTTCATTATTTTCTCAAATATT
TTTTCTGCATCATATTATTTCTCTTTGCCTTCTGAGACTTCAATGATGTGACTGTTAGACTTTTGGGTAT
TGACCTACAGATCTCTGACGCACTACTTACATTTTCTATTGTTCAAACTGGACGATATCTGTTGATCTAT
CTTCAAGTTCTCTGACTGTGTTCTCTGTCATTTTCATTCTGCTATTGAGTATATCCAGAGAATTTTTAAC
TATATTTTTCAGTCTAAAATCTTCACTTACTTCTCCTTTATATCTTCTTTTTTATCGCTGATACTTTATA
TCTTTCCATTTATCTCAAAACTGTTTGCTATTACTTGTTAGGGCATGTTATTGTGGCTGGTTTTTTGTGT
GTGGAGTTGGCATCTGTTGGTTATCTTTTCCTTTTCAAATTAAGACTTTCCTGAGTCTTCCTAGCTGAGT
AATTTTGAACTGTAGCCTTTGCAATGCTATACAGATCTGCCCTGCATGTGCACCACCCAGTGGCCAGTCT
GGAACCTGGGCCTTAATCTACCCCACAGTTTACTTCCTGAAGACTGTGGTATGATGTTTAGGATGAGATC
TATGCATGCACAGTTCAGAGGTGACCACAGGAGTCCATCTACAACTTTATGGAATTGCCTTCTTGAGCTC
CCTCCTCTTCATGACCCATTTCTCATTCCTCTACCCAAAAAGCTGGAGCTTCAGTTTCCTCACTCTGCCA
TATAGGCCCCACAACTGTGTCTGCCTCTGGGGCAAATCAGCAGAAAACAGAGAGAAAAAACAGTAAATAT
TTGCCCTCATATTCTTGGGATTATAACTTCTCTGGTAGAAGAAAATAGTTATGCTTGCTCCAGAGTTGTA
GGTGGCTGCATGGCCACCAACATCACTGCTGCCATTGCTACCAAGAGAATTGCCTGGGGGCTGAGTTGAG
AAATAACAGAATAAAGTGGGGGAAAAACAACAACAAAAGATTTCTTTCACTCTCTCTGACTCTTAGGGGT
TCCCTTTCTCACTCTGCAAGCCAGAAAGAGAGGATTTCTCTTGTGTGCTCTTTTTCTGCATCCACTGTAC
CCTTCTGGGTTTGGGAATGCCTTTAAGTCCAGTCTAGATGATACCAGAGGGGAAAGAAGAATGGTAAATT
CACTGCCAATTTGCTAATGCTTTAAAAATTCTTCCCACTCTTCCCCAGTTATTCTATTATTTACTTTGCA
GAATCCTCTAGTAGTTGCCCCTTACATTCAGTACATTGTTTATAATTGCATTCAGTGGGAGAGACAGGGC
TGAGTGTGCTTTACTCCATCTTGCCTGAATCTGGAAACTATCTGCTTGGTTTGTTGTTGTTTTGATTTAA
AGAAAAAAACAAGGATGATTTGTACACACAAAAGAAAGTTCTAAAACTAATTGTTTTTTTATTCAATCAT
CAGTCATGAGGCATTCACCCTTTCCTGGCAGCATCTGGTTGAGAATGTCATCTCCTTTTCCTTTGTAGGG
ACACAACTGACTGTGGAAGTCCACCCTCGAGATGCCATGCCCCAGCTGCTCAAGAAGTTCTCCTTGGCTA
AACGTATGTATGACACAGGTACTCATGGTGGAGGGCTGGCAGAGAGGATTGAAGGCAGCTGGTTGGGCCA
GATCCCAGCCCAAGCATCTTCTGAGGGAAGGAGAAGAGTGACATGGGCCTGCAAACTGGGAAAAGGCAGA
AGCATGACAGCTTTATCCTGGGAGACTCTTTTCTCTACACCCAGCTACCGCTGATTTTGCATCTCTGGAA
CTAGTCATAGGTAACACAGACATTTAGCTGAAGACACATTGCATCACAAGAGTGTTGTTGCTGTTGCTTT
TAGATACCATTTGTTTACTGCCAGAAAAGTGGCATGCAAAATGCTAAGTACTTTAACATTTATTAACTTT
TTAACCTTTTGATGTATACTAATATCATCCCCACTTTTTAGATCAGCAAAGTGAGGCTTAGAGAGGTGAA
GTAACTACCCAAGTCACCCAGCTAAGAAGTGGGTCAGAACCCAGGTATGACTCTGAAGCCTGTGGTCTTA
ACCACCATACTTTGCTGCCCTCAGTGCAGTGCACATAGGCCCAGGCCCAGGACGATGATGTCTCCCCAAC
TCCCTTCTCGGTCCTCTCCCTGCATCTGTGCACCTTATGGTTAAACAGGGACCTCAGGAAGAATGCAGGT
TAAGTAACAGAGGCTGCAGAACAACTGAGGCTCAAGTCCTCTTTAGGTCCATGCAGTGAGCCAAAGTCCT
CCAAAACTGTCTGGTCCTGTAGTCATCAGCAGTCAGGATCTCAAGAAAGTAGTCTACTGTAAAGAGGGCA
GGCTCTAGGGTCGACTGACTGGGTTCATGTTTCAGCTGTGTGACCTTGGGCAAGTGGCCTAACCCCTCTT
TTGAACTCAGTTTTGTCAACTATAAAAGAAGGAAAATAAGCGAATCTGCCTCATAAGATTATTATGAGAT
TAAATGAGAGAGTAAATATAAAGCCCAAGCCAGGCATAGTAAGTGTGTAACAAATGCTAGCTATCATTTC
GAACCTTTATCATTCTATCAAAAAGGTAGGTGGGAAAAAAATGTAGGTGTGAAATACAAAATGCAGGTGT
GAGAAATAAATGGAGCACTGTTTCTCAGAATAACCCATGTGTGGTTGCAATCTTCTTTGTTATATTTATA
GCCTAAAGTGGCCTTCAGGGGCATAAAACTACCTGCCTTGGCATCCCCACCTTCAACTCCCCTGATCCCA
TCAGGGACCCAAATCTTCCTGACCTGGTTCCAGATCGGCAGGGAAAGTTCCTTTCCTTTTGCCCAGGGAG
TCTAATAACTATTGGCTACAATTCTGCCTTTACGGTCTTAGATTTATGGGATTCAGTTGGCCTTTAATCC
TCCCAAGGAATAATTTAATTATGACCTGACCTTTCTAGGTTTTGACCTTCACACGAACTGCTTTCCCCTT
CCTGCATCTTTGTTTCCATCCATGGTCAGTTCTTATCCCGGCATTATCTGGCAGTGGATTCAAGAAAGCA
GGAAACTCCAAAAGAGGGCAGGAACACCAGGTTCCAGGAGGACCCAATGAATCACCCAAAGTGTTAGGTC
AGAGCAAATACTAACTCCAGTTGCAGAATGCAGCTCCCTTTCTTGTCAGCCACTTCCTGGTTCTTAGTCT
TCCTTCTGCCACACCAGGAAGTTCCTGGCAATTCAAGCAGCAATTCAGTGGTCAGTGGGGTGGGAACAAG
GAGACTGTGAAAAGGAGAGTAAAGAGGAGGGGTCAAAAGCAAGGACCACCAGAAGGTGAGTCATCAGTGC
TTGCATTTCTGTTTCTTCTGAAGTATCTATACTTCTGATCTTTGGAATATTGTAGGTTTACAAGGTGATA
AAAATGGAAACACAAGACCCCGGCAACCTGGAGGGAAAGATGCCCATGGTAAGTTCCAGAGTCTTCCAGG
ACAATTCTCAAGAGAGCACATGGGTTTAGCCTCATAGACTAATCTCCAATTGACAGTTGCTTCCTGGGCC
ACTATGCTGAGAGGCTGGGGCCACCTTTGGGTTCAGGAGGGGAAGAGTGTCATGATTGATTAGCAATGTC
TGCCATGGACACAGGTGGAGAAGGAAAAAGCTCATGCACCAGATGTCATCCTAAAGCTAAAGTATAAACC
CCACCAATAACAAGGCAGACCATTATAGGAAACATATTAAAATCGTAATTCAGGATTATGTTAACTTTAA
ATGGAATGCCAAATGTCTGTAAGGTAGGAGGCATGTGAATAAGCAGAGTGAAGCAGAGGAAGAAACACTG
AGAGGGATGATAGACAGCCTGGGTTGGAATCCTGCTTTTGCCCTTCATTTGCCTTCCCTGTGCCTCAGTT
TCCCCCTCAGTAGACCAGATGTCATCTAAGGCGGGGTGCAGCTCCATACTTCTTCCTATTGTTTTGCTTT
AAAATCAGAATAATTTAGTTAATGATACTGATGTTTTAAATGTATTTATGTAGAATCATACTTAGATCTT
ATATAATCTCCTCACAGGTAGATTTAATGAGGCATAAATCATGGGCATTTTTAATGTTCAGATGTTGGAA
AATGGAACCAAGAAAGGGGAAAACTAGTAATAGGATTTTCAATACCATTTCAGAAATGCCACCTTTATTC
TAAATAGGAGTGCAGGGAGACCTCATTTGCAGAGCTATACTGAGTGGAAATCTGTTCGATGTGTGAGTCC
ACTTTGAATGCACTTAGATCGCCCTGATGGCTCTTGTGAGAGAATGATAGGAACAGATTCTGCAGAGCAA
GAGGGCACTGAAAAGAGGCAAGGCTCAGCAGGGGCCTGCCTGAGTATCCCCAGTCTTCTCTGGTTGCATT
TGGACCACACAGCACAGCGGTTGATGACATAAGTCATGGTGGCCAGGCAAGAAACCCCTGGGCCCAAATA
TTGGTCAGCTCCCCAATAAGTGGGCATTGCCTCTCAAACCATGCTGGCCCAGAAGAGGGTCATGAAGGGG
CTGGAGCCAGGTCTAGAGGTGACACACACCCCTCCCCACCTTCTCCCCACATCTCTCAGGTCTAATAGCA
CCAGATTAAGGGACTCTCACTGACAGTGGCAGCCCTGACGACGTGTGGGCAAATGCACCTGCAGTGTCCC
CCACCAGGCAGGGAGGCCACATGTCCCTCCCTGCATGTACCTGCAGAGGAGCCCCACAGCCTGCACCCTC
CCAGAGAGCAGTGTGACGCTAACATCACAACATGATATGCTCCCAGGAATCCCACAGATGTGTGCCTGAG
GCCAGAGGGCTGGGTCCAGTCCCTCCCAGCAAGGTGGAAGTCAGAACCAGACTGGAACCAAGGTAGAAGT
GCAGGTCCATCAGGTGCTTTCTCACATAGCCCTACTCTTCCCAGACTTCCCTACCATACCCTGGGGTCTC
TCCAGCACACCTCACTTGCCCAGCACCCAGAGGCTCCCGACATGGAGGCTGAAGGTGGGTGGGTCCACCT
GGATCAGGGGCTCAGTTCCTCTTTGGTCCCAACTTCTTCCCACCCCCATCCTGTCTTCCCACCCAGCTTT
CCTGATTTTGATCTGCCTGACTCAGGGACCCTTGGTTAAGAAAGGCTCTGCCAGAGCCAAGGCTCTCTTC
TGGGCCGGGACAGGGGTCTGCTGTCCATGCACCTGTTGGTCTAACCATTGGCCAGGCAGCCTTGCAGCAG
CCTCCCAGCAATAGCTGATTATGGCTCTCCTATGTCCAGTGGGAAGCTTGCATGATTGCTGGCCCTGCAT
CAGCACTGGTCTCTAGTTTTCTCTCCTTCCTGTATCTCTGTCTCAGAGACCTGACTCGTGGTTTGGTCTC
TGGTCAGCAGGCTCCCTGTGAGTAAGGCTAAGCATAGATGAGTGATTTGGAGGCCCAGCCTTTCTCAGGG
CCCTGTTACCAGAACGCCATCACACATGGTTGAGATGCTCAGGTCTCCAGTTGACCAAGGGCAAAGGAGG
AAGCCTGTCTGGGTGCCAAACGCACCTCGCCCTGCAGGACCCAGATGGCCCGGCCTTTCCATGCCCTCCT
CCTCCACTGAGGTGTCCTTTGGCACCTCCACCCCAGGGCCGTGGCTGTGGCACAGACCTCCCTCCTTGAG
CTTGTGAGCACGAGCAGCACTGCCTGGACACTCCCCAAAGAGCATTCATTCCTGGGAAAAATAACAGGGA
ATCCACGGAAAACCAAATGGTGGGAAAAAGAAGATTCCATCATCAATATGTCTGGGAACTACTGGGTTAA
CTAAGGTTAAGCCCGTTTCCTACTTCTGACTTCTCAGAGTCTACAATAGGCGTATGTTCCCTGTAGGCCT
CTAAGAGGACTATGGGAGTTGCCATGGTTCCCAAACTAACTTGATCAGGGGACCCTTCTAGGAAAGTTCC
CATATAGCTCTTAGGCTCAATGTTTCAGGCTTTGAGAATTGTGTTAAAGGTTGGACAGTCACAGCTCCTC
AAAGAGACAGCAAGGCCTAGGCCAGGCAGCCCTCTTCAGTGGGTCTCAGCTGCTGACCAAGGTGCCCTGT
GTTTCCAGCCTACCCCTGGGATCGATCTAGCCTGAAATCCATGTCCCTGGACCTGCAGCAGTTTGAGAAA
TTGGATATCTACGCCTCACAGGTAGGACAGCTGGCCGCTCTGACCATGGGCCCCTTCAAGGTGGCTTAGA
GTTCTAGGGCAGCAAAACCTCTCCCCAGACACCTCCTGAACATATCCTGAGTGGCTTGGTTGGATGGCCC
TGGAATGCCTTTTCATAGCATATGGGTGGGGACAGAAGGCACTAGGCAGCAGCCCCTGCCCAGAGCCAGA
GGCCATTCCATCCCCCAGCCCACCCACGATCCTGTTGCTCCCACCCCATACAACACACCCTGGAGCTCTT
CATTGGCAAGCCCCTCTGGCCAGCCCTGACCTGCCTTCTCCATGCCCCTTTTGGCATTTCTGCTCCTAAG
CTATGCCTGCCTCCAGTCCATGGAAGAAGGGAGGAGAGGATGGGAGGACCGTGGATGCAGATGGCCAGGA
CCATTATAATCACAGCTGTGCTCACCTCTGAAGTGCATGAGCTGCCATGCTCTCATGCCTCTGGCCCTGA
GCATGTCTCAGGTGCAGGTGGGAGGAGCAGATGAAGAGCTGAAACACTAGGGACATATGGAAGTCATTGG
AGCCAGGCAGGAGCTTCAGCACAGAGAGTCTGATAGGAGGAACAAGGTGTTGAACACTTTTGTGAAGTCA
GGACATTAAGTGGTCTTAAAAAGGCCACCTTATAGGTACTCAGGAACCTTGTTTGTTGAAATAATGAGTG
GATGGTGAGTGCATGGATCAAGAAAGCCAGATGAGAAGCAAAGACATAAGTTGAGGACATCCTTCTGTCC
TGGCAGAAGACATGACCTGGATTAAGTGGAGACATTGAGGAAACAGAGGAGTGGAAAGGCCAATCCTGAG
CTAGGGGCAGAGGGGAAGAACTCTAACACCAGTGGGTGTGGAGTGGGTGGGAGGTGGGGGAGAGGGGCTC
CCAGATAGCTGGATTGACTCCTGGAGGGTGCATCAGTACCTCCTCACGAGGAGCAAGGGAGAAGGAGCAA
GTTTTAGGGGATGAGTGTGTTTGGGATATGAGGGGCTCTAGTGTCTGTGGCACATCCAGGGGAAATGCCC
CCTGGGCAGCCAAATATACAAGCTTGGAAAGGTCTGGGCTGGAGACACTGATTGGTAGTCATCAGATTTC
GTTAAGAGCTGAAGCCATAGGACGGGCATCATATTGCAGGGGTGGAGGAAGCCCATAGGGCCTGGAGCCA
TCAAAGGGGCTGGGGCAAGGCCAGGAAGGGTCAGAGTTCTGGAAGGTCCAGGGAGGGGCTCCCTGGGGCA
GACGTGGTCAACAGCGTGAGCTGTGCTATCTGCTCAAGTGAGATCAGGACAGACGCCATGTCGGCTACTG
CAGCATCAAAGTGCACCTTACCTCCAGAGGGGCAGTGCACAGGGGCTGGAGGCCTCAGTCCTCAGGTGGG
TGGAAGAGCAAGCAGGAGGTGAGTAGTGGGGAGTGAGGAGAAGATCCCACAGGAGCCTGCAGAGACGGGG
AGGCTAGACATGAGCAGAACTGAGGAGAGTAGGGGGGCATCCTAAGGTGGGGGAACAAGGGGGCATGGAG
CAGCCAGGAAAGGAAGGGAGGAGGTGAGGACATACGAGAAGGAGGCCATTGATTGAAGAAGGGAAGTCCC
TGTAAAGGCAGAAGACCTGAGACATAGAGAAGGCTGAAGCAGGAGGGGAGGATGAGACTCTTAGTGAGGA
AGTCCCACCTGGCCTGCCGTGAAGTGGAGGCCAGGCCATTACCTGTGAATGAGGAAAGTGGGTGGGGTCT
GGAGTCTGCAAAGGGCCCAGATTTTTGTAATGGCCATCGGAAGGGATGGGTCAGGCTGCTGGCCATACTC
AGAGAACAGTGCCAGGAAGCCAGCGGAGGCCAGAGACCCAGCCACAGAGCAGGGAGTGGCCAGAGCAAGC
CAGGCCCAGGTGGGGCCAGGAGGTGGGGACCAAAGGACCAGAAGCCATGGCACTGAAGTAGAAGGCTAAG
TCAGGACTCCAGGCTGCAGTGCAGGCCTGCAATGCTGAGTACCATTCGGGGCCCTGGGAGAGGGACAAGG
ACAAAGTCTGCGGTGTTGGAGCTGGACGTAGAAGAAAATGAAACAGAAAGCAGCTGAGAGAGTAGAACAG
GGGTCCAGAGGTCTCCAGAGGTCAGAGTGCCAGTGTGCTGGGCCAGTGGTCAGCAGGTGAGTCCTGGAAT
CTCTTGTGAGCAGTGAGATGTTTCCAGCTAAAAACAAGGTCCTGGAGTACCCTCAAGCTGGGAGCTGAGG
GGAATGTGGGCACCATTGCAGGACCTAGTGGCCCAGCTTCTCTACAGAGATTAAACATGGTCAGAGCCAT
CTTTCCAGCAACCTCAGCCCTGTTTCCCTGAGGGCCCCTACGAAGAAAGCTAATTTGTTCTTTTTTTATC
ATAAAATAACATAAAATTTGCCATTTTAATAATTTTTAAGTGTACAATTCACTAGCATTAATTATATTCA
CAATGTTGTATTCTCATCACCCAAAACAGAAACTCTACCCATTAAGCAATGAAATTCCCATTTCTCCTCC
CCCAGCCCCTGGTAATCTCTACTTACTGTCTATGAACTTTCCTATTCTAGATATTTCATATAAGTGGAAT
CATACAGTATTTGTCCTTTTGTGTCTGGCTTATGTCACTTAGCATCATGTTTTCAAGGTTCATCCATGTC
GTAGCATATACAGAACTTAATTCCTTCTTTTTGTAGTTGAATAATATTCCCATGTATATATGGTATATAC
TCTATTTTATTTATCCTTTCATTCATGGATGGATGTTTTTTAACCTTTTGGCTATTGTGGATAGTGCTGC
AATGAACATTGGGGAAACAAATGTCTGTTTGAGTCTCTGCCTTCGGTTTTTTTGGTTGCGTACCTTGGAC
TGAAATTGCTAGATCATATGATAATTCTACATTTAACTTTCTGAGAAACTGTCAAAATTTGTCTACAGCA
GCTGCACAATTTTACATTCCCACTAGCATCGTACAAGAGTTCCAATTCCTCCATATTCTTGCCAACACAT
TATTTTCTGGTTTCATTTTTCAATAGCCATTTTAGTAGTCTGAAGTGCTATCTCATTGTGGTTTTGATTT
ACATTTCTCTCATGACTAAGGATGTTGAGCTTTTTTTTATGTGCTTATTGGCCATTTGTATATCTTCTTT
AGAGAAATATCTGTTCAAGTCCTTTGCCCCTTTTTGAATTGGGTTGGTTTTGTTTTTGTGGAGTTGTAGG
AGTTCTTTTTATATTCTGGATATTTAACCCTTATTGGATATATGATTTGCAATGTTTTCTCTCATTTTGT
AAGTTTTCTTTCTGCCTTCTAAATGATATCCTTTAATGCACAGAAATTTTTTATTTTCACGAATGCCCTT
TTATCTGTTTTTTCCTTTGTTGCTTGTGCTTTTGGTGCCATATCTAAGAATTCATTGCCAAATCCAAGGT
CATGAAGATTAACCCCAGTGTTTTCTTCTGAGAGTTTTATGGTTTAAGCTCTTATATTTGAGTCACTGAT
CCATTTTGAGTTGTTTTTGTATATGGCATGAGGTAGAGATCCAAGTTTATTCTTTTGCATGTGGAAACTC
AATTGTCCCAGCACCATTTGTAAAAGAGACTATTCTTTCCCCACTGAATGGACTTGACACCCTTGTCAAA
GATCCACTAACCGTAGATATGTGGGTCCATTTCTGGACTCTCAGTTGTATTCCATTTGTCTATATAATCT
TCATGGCAGTATCAAACTGTTTTGATTACTGTAGTTCTACAGTAAGTTTTGAAAATGGGGAACTTGTGTC
CTTCAAATGTGTTCTTTTTTCAAAATTGTTTTGGCTGCTTGGAGCCCCTTGGAATTTATAGGAATTTGAG
AACTGACTTTTCCATTTCAGCACAAAAAGCTATTGGAATTTTGATAAGGATTATGTTAAATCCTTAGTAT
TGACCACGACCAGGGGCTGCTACAGCTGTCCAGATGCCTGCCTGGCTAGCCCCCAGATCTGCAGAGAAAA
GCTGTGGTGGCTCTAGGGCCTGTGATTGAGCTGGTCAGCACCACCTGCCCTGTCCAACTTCCCCCACCTC
CTCCCAATTTGTCCTCAGCCAGCCTTTGCCCCTCAGGACCTCTCCTCCAGCTTCTCTGCCTGGAGGAAGA
GCAGCACTGGCTGTAGGTGCAAGATGCTGGGCCCCCAACAGCCCCTCCTCGAGGCCCTAATACAGCACAC
CCTTCCTGCTCCCCAGGTGACAGCCAAGAGTGGCCTAGACGAACTGGTGAGTGACCTGCTCCAGGAGGCC
CACACTGACCTGGAAAGGGTCCGCGCCATCTGGATCTGGATCTGCCATCACATAGGTAGGCCCCCCTGTG
GAATTTCTGGCTCTAGAATCCCTTGCTTCCATTCCCTGCAGGCCTCTGCTCCTATCAGCTTATTCACGTG
TGCAGAGTGACATGAAAGGTGGGGGACAGGGACCCAGAAGACTGGACTGATGAACCAGAGAGTAACCAAG
GAGGATAAAATTCACCATGTGCTATGAGTCTCCATGAAAGTTAGTTGGAGGAGAATCCCTTGCAGCCTTC
TGCCCATAACCAGTGCCCAGAGCTGGAAACTTCATCCAGCAGCAGCCTTCCCTCTGTCCCAGCCTTCCCT
CTGTCTTATCTCCACAGAGATGAGAGCCCAGAGCAACAGGTCATCTCCAAGAAGGATTACCATTGGTGGG
GAGAAGTTGCAGCTGTTTGAAAAGTGATTGCAATACCAGAGGGTTCATTAAATACCAGGGCCAGGAAAAG
CGGGTTCTCCACTTCCTGCTCCCACACTAGCAACATTGGCCACTGGTCCCAGCGCAGTCTCCAAGGTTCT
TGGAGCCACCTGAGGGACCTGGGCCTATGCAGCTGCCCACAGCTCTCCACAGGATGGCCTTGGGAAGCTC
CCGGGTCAAGCAGAGCTGCAGAGGTGGTGGCTAAGGGATGCTGCCCCTTGCCGCCTTGCAAACACAAATG
AGAGTGGGCAGGGCCAGATCACGAGGTGGACTCCTAGTCCTTTGACATAAAGACCAGCCGGGTCTTTCTC
GACCTGCTGCCACTGGGCTTTTCTTTTATTCTTCTGGAACAACAGTTTGGAATGCAGCTTAGAGAATGAG
CTCGCCTGGCACAGGTAGGAGGTCCAAACTGAGGACTTGAGAGACCCCCAGCTCAGCTCCCATCCCAACT
CCACCAGCTGTGCCTGTGCCTTAAAGGGGCAGGGTCAGAAATGGGACATTTATAGGACTTCGTTACTGCT
CTATCCCCATGCCAACACACACATACACAGTCTCAATGGGTCCTCAATAGCTACAGAGACTTCTGCCTTA
GGGCCTCACCACAGGCCTCAGGCTTCTTTGCAATGTCTTCATCCCCTCCTTAGTGAAGTGACAGGCTGTG
GGAGAGCAGCCTTCTCCACTTGGAGACGAGCTCTGTGTGCCCCATGGGTCAGCATGAGGCCTTCGGGCTG
CAGGAAAACGGGATGAGTGATTTCACTGGAAATGTGTGCAGAGCTGTGTGGTGGGGATTAGCAGAGAGAG
CCCCATGCCTCTGCACTGTCCTGGTCCCTTCTCCTCCTAGCCCCTCCTCAGAGGCTTGGGGAGCCAGGTG
GCCCCCTCTGCCAAGGCCTGCTCTTCCTCTCACCACTTGCACAAAGAGGTGATAGTTCCCAGATCCCATT
TGTGCTGGGGCTGTAGTTCCATTCAGCCCACTGTAGTTTCCTGGGGCTGGTGATGGCCAGAGCACAGCTG
GGGGTTGGGGTGGAGTCTTCTCGCCTCCCAGGCTCAAGCTCTTCATGATCCCCTCACCTGCCCTATAGAG
TATGACATTGCAGCTGCTCAGGAGAAGGACCGCCAAGCCTTCAAACCCACTGACATCCTGCGGACCCAGA
AGACCAACTGTGATGGCTATGCTGGCCTCTTCGAGAGAATGTGCAGGTACGAGAGACAGGAGTCCCCATG
CTGACCACCGGACCCATCCCTCTCCCTCTTCCACAGTGCCATGTGCCTTTGCCCACATGCCAGGCCTGAA
ACCTGAGTTTACAAACTCTGCTCTGGAACCCAAGTGAGTCCTGAAAAGGTCAATTTCTCCCTTTTTAACA
CCAGCATAATTGACTTTGTACCATCTGCCGGGCTTCGCGATGCATTAACATAATGGTAGATGAATGCACA
GAGATAATTTGGTTATAAAAAGCCCTTTTTCCGCCTTCTCCCATCTCTATGCAGCTGAAGGTTAAAAGAA
TGGCAGGCCTGGGAGCGGGAGGACAGTGACTGCAGATGCCAAGGGGACTGTGAAAGGCTGTCCTCAGGGC
TGACACAGGGAGCCTCTTTTGGGGGAAACAAAAGATCTCCCACCTGAAAAGATTTGTCAGTTGGACAGCT
GAATCACTACGAAGCGGCTACTCTTGGAGTCGCTGGGCTCCATTTTCAGCACTCTGGGAGTGGGCATATG
GCCAGGTTGCTGGGGTGGGTGACAGCTCTGGCCTGAGCAGGAGAACAGGCCATTGCCAGTTCCCAGTGAC
TCAAAACAGGCTCAAGATGTGGGGTGGGGGGAGGGGATGGCCACTGACCCCCACAGCTTCCTGAGCCTAC
CCTCTGAGGTCTGCTGGGTTCCTCCAGAAAACAGTCAGCTAGCCCTGGGGCCATCAAAGAACCCCTCCCC
AGGGGGTGGATGAACAGCGGCCCACTGTCTTTCTAGGCTTCTGGGAGTGAGTCTTCTTAGGCACCCAGAT
GTCAGCCCACCAGAACTGCAGGTGGCAATTCATCAAACCAAGTGTAACACTTAGTCCACAGGCAGGCAGT
TGTGATGGCCTCAGTGTTGCAGAGGCGGCAGCTGCCGAGCACTGGGTGCATGCAACCTATTCCTGGCCCA
AACCCCCTGTTGTGCTGCCTCCTGCCCCCATCACCACTGCCAAGCACAGGCACCTACCCGGCTGCTGAGT
TACTCTAAGCCTCCTGCTCTGCTTCCTGTACTTCCCTTTTCTCACTTTCAAGGCCAATCCCTGAGACCAC
AGCCCCAGACGCAGCCACGGTGGGTCAGGGTACAGAATATGCCACCCAGGCTGCTTCCCACCCCCTACCC
CAGACCCAAGCATACCTCCTGCTGCCTGCAGGCCTCTCCAGGAACCTGGATCCTCACTGGCCTCTCTGTC
CAGGCTTCCCTGGTCCTGGGTTCCTAACATCACTAAAGGATCACACTATCTAGCAAAGTCCTCCAAAATC
TGACCTCAGCCAAGTTTCCAGCCCTGCACTGTGTGACACCACCTTCCTGACCACCTCTGCATGCACACCT
GCTCCCTCTCCCCAGAGCTCCTTCCAGGCCCCGCTCCAGGACAGCCCACAACCTTCCCCTTCCTTTGTAC
CCCCAGCCCAGTTGCACACCAAACTCATGTGTTGCCTTATGCTTTCAATTAATAACATTTTTAAATTTAA
AAAATACCATTCATTGTAGATGGTTTATAAAATGCACTAAAGCAAAAAAGAAATTATTACTCTGAATCTC
ACCACTTACAGCTAACACTGGTAACATTTGGTCATATTACTTTCCAGACTGTGTTCTGTGCATACAGGAA
TTACCTGTGCTCGGTTGTTGTCATACATCCTATCGATTGTATACGTCTATATTTGCCTTTATCTCCTCTC
TCCTTCTACCTCCCCTGCGAATACCCATCTCAGAATCCTACACCCAGTAGGCCCTCAATAGACATATATA
GAAATGACAGGATATTCCTAGCAGACTTATGTGGAATGGAATGTGGAATTCTCTTTTAACTGATGAAATA
GCCACAAGTCTCTTAGCTCCATTTGTGCCTAGCATCTTTCAGAACATGATTATTTTCCTCTGATTTTACA
AATTTGAAAAATGCTTAGCATTTTTTCCAAGTTCCAGGATATATTCCTCTTTATCCAGTATAAGCTTTCT
AGCATACTGTAGAAGGAATGCCAGATTTGAAGCTAGAAAGACCTGGGTCTACCACTTAGAGTGGTATCGC
CTCCTTGATCCAGTATAAGCATCCTAGCATCCTTTAGAAGGAGTGCCAAAGTTAAAGCCAGAAAGACCTG
GCTCCCAGTTTTTAATTTACCACCTATTAGTTTTGTTCTTTGTGGTAAGGCCCTTAACCTCTTGCAGCCT
CAGTGCTCTCATCTATATATTGGAGACTAGATTGCCTACCTGGCAGGGCTATAGGACTCAATGAGACTGC
AGTTTGGGGGTTACCAACCCGATTTTGGGGTGGGTGTTTTTTGACCTATCTTGTCTTTCTTGAGTATTGA
TTGGAGTCCTTTTGCTCTGTGGAGTTTTCCTACAGACTCTGCCAGCCTACATAGATTCAGAAAATTGACA
CTTGCCTTGAGTGAAACTGTCAGCTTTTTGCCCTGGTCCAGATACTTAACAATGGCCCTATTCTGGCCTG
CAGCTTATCATTAAAACTCTCCCTAACATTTTGCATCATTTAAAATTAGTTCCAACATGGTGATTACCAT
GTGCAAAATTCCTCACACTGCCTGAGATGTGTAGCAATAGCTCACGGTCCCCTGAATTCCTTTAACTGTT
TCTTTAAAGATCAGTAACCTATCTATGCATCCCTCTACTAACTCTCTTTACCTTTAAAATAACTGCCAAA
AGTTTGGTAAATTAATTAGCTAATTCCTTAATAACTGAGGACATATAATCCGCACTTGCTGATTTATCCC
TTCTTGGTTGTCAGGGTGGACATCATTACTTTTTGCCAGTCCATTCCACCTCCCTGAAATTGTCTCATTA
AAGACTGATTTAATAAAGTAATTTGGGGAGGAATTCAGATATTTTCACAACATCATTCAGTTCCTTCTGT
CTGTCCCAAGTCACTGATGTGGGGTTTTTTGGTTAATGCTATAGCTTGTAACTGCTGGTCCAGTTTCCTT
CTGACTTTCTATCATCCAACATTAATGTCTCTCTTTTTGCCTCAGTTGCCTGCATCCTTAGTTGATTCTG
CCTAATTTCCTCGCAGGAGCCAAGCTTAATTCCCCAGACTGCTGCTCTTGAGTCGGCCTCCAGTAAATTC
TCTTCCCGTTAGCATGGCTTTAGCATCACCAGCTAGCAGGTTTCAGAGACGTTGGATAAAGCCAGGCACT
TATTACGAGTTTGAGCCCTGACACTGAGGGAGTTGGTTGTCACCAATCAGATGTCAGCTGGCTTTCCACT
CATCCTCCAGCTGTCACTGAGAGCAGGGGCAGGAGTGTGTGCAGTCAGATGGCTGCTTCTGGTTAGCAGT
ATGTCCCCTATAATCCTAAAACATCACTTGGTTCCAAAGGCCCCAAAAGATAGGCAGTGGCCATCTAAGC
CTTCTGGGAGATGCAGATTCATTGGATAACCTTAAATTATCACCAAATCTGATGCTGGTGTATAAGGCCT
CTGAAAGGGACATCTTAGCTCCCCCACCCTTATAGCTACACCAAAGGATGGCACTCACAAAGATGAGTTA
TGTCACTGGTGCAAACCACCTTTCCATTTCACTCTCTATACCACCTACAAGTGTATAGATCAAAACAAAA
TCTTGATTTACAAAAGAGCTGGTGCAACATTGTGCACATGTATCCTAAAACTTAAAGTATAATAATAACA
AAAATAAAATAAAATAAAAAGAAAAAAGAAAAAAAATTAACTTACCATGTCAGCTATGAGGAATATGAGG
TCAAATGATTTTTCCATTTTCCCAATAGATGTTTTCACACGTGAAAAAAAAAAAAAAAAAAAAAGCTTCT
TGAACTATGCATCAGGACAACTGCATCCACTGCTGCTCCGGCCACTGGCTAGCTGTGTGTCCCCAGGCAA
GGCAGCCTCTCTCTCTGAACAGCTGACGCCTCCCTCACCTTCAGAGAGGAGGCCTCTGAAGTCACGTCTC
ATGAGGTTCTGCATGGGGCCCTGGAGAGAGAATTATTATCAAGATTACTAGTTATTGACGTAGCTGTAAG
GGTAGTGTGTAAGAAAAGGGAAAAGAGAGCACATGGGAGAAATATAAACACACTGTCAGATTATAAGAAA
TCAAGTACATAAATTGTGAAAGATAAGTCATGGTTTGTTTGTCCTGCTGCAGTATAGAAACCACAGTATT
TTCAACAAAATACCAAAAGAAAAGCATACTTCATAGTCTCTCCTACAGGCACACTCCCTCCCTTTTTCCC
TCACTTGGCTCACACATCCTAGGAGAGGTGATCAGATTTGGGAAAGTCTACTGCGGTCAGAGCCCATTGG
TGTAGGGTGCACCTGTCCACCCAGATATTTCAGCCACACAGCTGTTGGGGCCTGGCCTTGGTACTGACTG
TAGGAGCATCATACAGTTCCTATGGCAGGGCCTGGACCATCTCAGTATAAAATAATACCAAATTAATACA
CTAGGATACAGTGCACCAAACTGGAACACACAGATCTTTTTAAATAATAAGGATAATGCATTCATAGAAG
ACTCAGCAGCTTCAGGGTTCATCAAGGGTAACCCAGGTAATGACATTGCCTTCTGCTTGGCTGCCCCTCT
GTTCAGTCTCTTCCCATCAGGCCACATTTGGTGGCCATGTGGTCCTTTGCAGCCCTTGGGGCTTCTCTGA
TTGGGTCCCTGAGCCCTGATATTGCTGGAGAGTGGGGGCTGCAGCTTTCTCCAAATCCTCCAGGGTGCTC
CTTGGCAGCTTGTTCAGGGCATCTTACATCCATGTTTTCATTGTGGCTTGGGATATATATCAGCCATGAG
CTATGGAGGCAAAATTGAGGTTGTCAATTCTGTAAAGTAGAAGCGAAGATTCTGCAGTCTGAGGTCATTA
AATAAATCTCCTTGGGTGATATTTTAGGCTCTGGGATGGCTGGTCGGAGCTCTTCAGGAGGTGGAAATGT
TTCTAGTCCTCGGCCCCTCCTTCCGTGCTCTCTCAGCTCCTGCCTGCAAAGTTAATTCCTCCACCTTTGG
GCTCTCCAGGTCTTTGCTTATAGACTGGGATTTTATAAGCAAAGTTCTGGAGACAGTTCCAGTGACAGTT
CCCATATCCACCCTCCTTCCAACTATCAGCACATCTAGGGCAGCCACTGAGCCTTATTTTGGGGATATGA
TATTTTTTTATGTTGTTACATTGTGTTGCGTTGTATGGTGCCATGTCATGTTATAGCATTCCCTGCTACA
GTGCTTTATACACAGTGTGTGCGGCCAGTGATCTTTTGATGACATGAAAGAGTTGTGGTTCATCCTGGCT
AGCAAGAGTGGCTGGAGACAAAACTCCTGTGGGTTCCACAATCATCGTCTTATACATACAGAGGATTTTG
CATGAGAATTATGGCATATTAACAATAATGCTTTACATTATGACCTTCTTTGTCCAATTCATCTGGCACA
TTGCTGCCAGGTAATAAAATATCCTCATATCAGAGTCAAGCATGGCATAAGCTTATAACTGAGTTTATAT
TTTCATCTTTTTTCTGCCCCAGGCATTCCCCACAGCCTTCCCAGCTTCTCCTCAGCCTGTCAGAGAAGAA
TCAACCCCTGGAGATCATTTAATGCCAACATTTCTCTCTGCCCATGATAAGCCTGGCAAGGTCACTCTAG
AGGTGACCACCAGACCTGGGAGGTGACCTTATTTCCTGGCTCAGGGGCCATCACCCCCACTCAGAGCAGT
ACAGGTGGACATCCCTGCCCCTTGCTTCTTCCTCCTGCTCTCCTTTCCAGGGCTGGGGCAGGCTCTCGCT
GCTGCTCCCTGACTTGGTTTTTCCAATCTTGCCTCCACTTCACTCCCTTGAGGGCAGGATGTCCCCCTGG
GCACACGGCCTAGGACATGGCTTCTCCCAGGAGGACATTCCATACCCATTCTGATTTACCCCACCGCCTG
GAGGCTGCACAAATGCTAAATTCCTCTCCTTCCTTCCTTGACTCCAGGTGGCTCTGAGCAGAGGACAAGC
ATCAGTGGGCAGCATGTCCAGAGGTCCACAGCGCCGCTAAATCAGTTCACCTTAATGCAAAACGAGCCAT
TCTCCAAGCCCCATTGTTCCCAGGACTCCCCTTATCCCTCCACATCATCCTAGAAAGTGATATGCACAGA
CATTCTAGGCAGCAATGGAAAGTAATTTCCTGAAGAATATAATTTTAAGTAGTATTTATGGCTTTTTTTC
CAATTCTCAAAGTAACACATAATCATTATTAAAAATTTAGAAAATACAGAGAAGTTTCAAGAAAAACATT
AAAATCACCTATAATCCCATTGCCAAAAGATCAGGTGCCTTTTGTGCCATCTCTCCCCCTCTCACTCCCT
GCCCCTCCTTCTCTGTGCATGCAGGCAACACACACACACACACACACACACACACACACACACACACACA
CAGCATCGGCGTGACATTGTGCTGACTGGAGAGATTGACATGCTGTTGTCAGTTCACATCGTGTCAGGGG
CACTGCTTAGCTCACTAAATATTTTTCACAACATTTCTTCTCCTCACCCAGTCTCTGAGTAGATGGGGCC
CCACCTCCTATAGAATGTCTATCCCACCCCTACACCTCTACCCACTCCTCTGGCCTTGCTTTCCTCTGGT
CCTTCTCTGCTAGCTGTTTCCTCTCAGACTATAAACACATCCAGACTTCCTCATCCTGAGAACAACCCCC
TCAGCCTTGCATCCCCTCAGCACTCCCCACCACCTTCTGCTCTCTGCACTGCAGAGAAATGAGGAGAGAG
CACTCCAGGTATGCAGGCTGCCCCTTCTCCCCTTCCTTCCACGGTCTCCTCAGCTACTGCCTCCTTCTGC
CCTGGAGTGGCTTATGGCCTTTGCCAGCAGAAAATCACAACACTGGGATCTAGTTTCTCTTGCAGTCTGT
CCCCATGCCCTCAAGTGCTCCCAGAAGCCATCCCACAGAGCCAAGCTGGTATGTTCTTATGGGATTCCTT
AGAGGGCTTAGGAGGCCTGAACCTCCTTTTGTGAAGCAACAGGCAAAAGGGCGGGGGCTAGAAAACTTCC
AAGCCCCTAAGGAACCTCAGCACTAGTGGCCCGGACTTTCCACAGGTGAGCAGAGGTTAAGGCAGACATG
GTAACACCTCATCATTGCATGCATGTGAAGAAACACTGATGGCTGGGAAAGTAGAATCTGTTAACCCTCA
GGACATCACATTTGATCCCAATGACTCTCTCAGCAAGTGATATGTATTAGTAATATTTAATCCAATATTT
TGCTAATTTTAAATTACATGTTCATTTTTACAAGGAAGATTCCCCCAATTCCTATAGTAGATGCATGGCC
ATTTCACCCCATTCCCCTGGGAGGCTCCGTGGCCCTCTTGTTTAGAGCAACTGAGGCTGCAGCCCTGGTC
TGTGGAAGGGCCCAGCCCCATGGGCCCATCTCACTGAAACAGACCCCACCAACACCCTGGACTGCAGACT
CAGCTCAGCTGAGTACAGCACAGGGTCACCACTGAGTTGGGGACTGAGCAGCTTGTGTGGTACTCAAAGG
AGCCCCATCAAAGGGGCATCAAGCACATCCTCGGTTTCACAGGTTGTGTATCTATTACACCAATTTTCCA
TGAGTGCAAGGAAAATGTCTTGAGGAAGGGGAACCTTTTCTAATTTACACAAAGGCCCTAGGAGGGCTAG
CATGGGATGTCCCTAGGTCAGACTAGCAGTGAAATTGTCTGCACAGTCCATCCCCTTGGCTGTCCTCAGG
GGAGCAGGGCTGCTGAGGAGCAAGACCAGCTGGCCATGAAAGGACACCCAGGGGTCTTGCTTTGGCCTTG
GACTGGTGCCCACAGCCCAGGCCTGGGCCTCAGGGTTTTAGCTCCAAATGTCTCATTTTATATCTTCCCC
AGAGGTGACCCATGCCCATCTTCCCATCCAGGTTCCCAGAAAGCAGTGCCAGCTCTACTATGAGCAGTGA
AGGAGCACAATGTCAAAAGGACCCCAAAGCTCAGGTTGACTGGTCGGCATTTCCATTCCCCTCAGGACAG
CCACAGGAGACTCCAGAATTGTGGCCAGGGCAGGCAGTCAGCAAATGACCTTAATGCACCATGATGGCTC
TTGCACCATGTTCACAAAGGACAGACCCACCAGTCAGACCTGAGCAAACTGGCAGCTGGCCAGTCCTCCC
TGGGCCAGGGCTGGAGACATCCACCGCCTGCCCCGGGGTTCCCCTGCAGGCATCCTTGGGAGGAATAGCA
GGGGGGTAGGGCTTGGCTTCTGTACCCTCACAACCAGCAACCCCACAACACCTCACCCATGTCACGAGAC
CCATCTCTTCAGTCGCATGTCCAGAAGCGCCCTGGTTCTCCCAGCAGCTGTGCTCGCCCCTTACATGTAG
GTTTTGTTGCAGGAACATTGTCATTTCCCCTGCTCCTATTTTCTTGAAGCCAGCCTCACGCTTGCCCCCA
TTTTTTCCTCCTGAAGAGCTGCTTGAGGAGCGTGTCAAGCAGGCTCTAGGGATGAACATTTCAGAAGTGA
TCTGAATGGTGTTGGTCTCAGGGGAGGAGCAGACAGCGGAGACTGCAGCCAGTCTGTACCAACAGGACCA
GCCAGCACTGTATCTCCCTGTCCCCAGTGCCCCGAGATAAAAGCCATCAAGGCTGTTTATATGTGAGTAC
AAGAAGCAAGGAAAAGGATAGACAGGGAGGACTGATGGGCAGCAAACATGCCTGAAACCTGAGACCATGG
ACAGACTTGGAGGCAAGCCACAGCGGGATCCCCCTCTCAGACCCCCTGTCCTGCCCCCAGGCTCGCCGGA
GTGCAGTGTATGACCGTGCCTGGCTACTCCAAGGGTTTCGGCTACCAGACAGGGCAGAGCTTCTCGGGGG
AGTTTGACCATGCCTGGAATGCTGTGTACCTGGAGGGAAGATGGCACCTGGTGGACAGCACCTGGGGCAG
CGGCCTGGTGGACACCATCACCTCCAAATTCACCTTCCTGTAAGTACTTGTCTCAGTTCTGCTGGGCGTC
TGGCAGGTGGAAGTGGAGGATGTGCCAGGCAGCATGAAGCAGCAGGTGGAGAAGACCAAGCGGGAGGGGA
AGGAGAGCCAGGAGGCCCCATGCCCACTCCTCCTCGGCTCTGAAGTGGGCTGGCTCCAGGCCAATTGTGA
GGGCCCAGACCCTTTTGTCACCTCACCCTCCACTGACAAACTCCCCACTCTGACTCCCTATAACTCCCTC
CACCAAAGCACTGAAAGCCTGCAGCATTTGCCCCCAGACCACACATGAGGAAAGGTGGGCTAATAGCCTT
CTTTAGTTGTTGGCAGGTTCCCTTCCAGACTTTGGCCATTGACCACATTACCAGGCACAAATGCCAGAAT
GATCTCTTACTGCTTTGCCCAGACACCAGGCTCCTTCTCCTCTCAGGTCTGAGTGAGACCGTATCACATT
TCTGAGGAAAGGGCTGTGGTTTTTGGGGAAGGGGTGCTATTGGCGGTGCAGGGTCACTGTGAGGCACAGT
GGGGCAGGGCAGCCGGGGTGCAGAGGACTGGAGCATTTCTGTCCTCTGGGGCTCTGGCCAATCCTGGCCT
TAACCTGAGGCTACATACAATATTCTTCATGGATCATGATGTTATCAGCAGCTCAGATTCCTTTTTATTC
AGAAACATGAGTGTGCTCTCTCGATTTTTGCACCTTAAATCTTGACACTCCCACACATGGGACAAATACA
AGCCATGTGCACACACATCCTACACGCAGGGCTCGCGTACTCACAGATACCCTCTTTACCTCTGTTATTC
TCAACACTGCTCCTCTTAGGCAGAGCAAAGCCACCCCCATCTCAGGAAGGGCCTCTCTGTTCTTCTCTTG
ATCCCCATTACTGACATCCAGAGAGGCAGCACTCCAGTCGGCCCTGGAGAGCCACAAAAGGTCCCCGCCA
GCACAGCTGCACAGCACAGAAACAGGTTGGGGTAGATGGAGGCAGTGAATTCAAGCACTCTGTCTCCCTG
CCTTCCCACGACCCCACTGCGTGGTCCACAGGCTATGCCTTTCCCCCCTTTGAACTTCTGATTGTCTTTG
AGCATGAGGCAGTCAGTTTTCCCCTCGACAGCCTAGGGCACTGAGTGCAGACCACAGTTGCAGGCTGTGG
CCTTCCCTTAGAAGGGACCTCAGACCCTGCCTTGACTACAATTTCTGGAAGCATCCACTGAGGAGGAGTC
TCCAGTGCCCATGTTAGGGTCGGAGATGGTGCCTCTTCCCATCCAGGGTGTCCATAGGACCACTCCGGTG
GGTGTATTGGGCCCCTGCCTCCCCTGCATGGAGCTGGCTGCTAACCAGCTTGTCTCTGCTCCAGCTACAA
TGAGTTCTACTTCCTCACCCACCCTGCACTGTTCATCGAGGACCACTTCCCAGACAACAAGAACTGGCAG
CTGCTAAAACCTCCTCAATCTCTGAGGCAGTTTGAGAACAACATGTATCACAAGAGTGAATTCTACAACA
AAGGGATGCTGAGTGCCCACCCAGAGACTTCCATGATCAGAACAGGTGAGCCCGGAGCAGGTGCCAGTGT
GCTAGCAGCAGGAATGGAGCATTTCGCACTGTCCTCCAGGAGCCCGCCTGAGACTGCGCTTTCAAGGCAT
GCCCTTCCAGACGCATGGCTTGTGAGCTTAGCACAAAGCTTGTGTGTGCATGTGGATGAGTGGAGGCTGC
TGAGGCAGCCCAGGCCATCAGGGCAGGGAAGGGCCATAGGCCACATCAGCAGGGACAAAAGCTCACTCAC
ACTAGCATAAACCCAGAAAGGGATGTGTCTGAAGGCTGCAGGTTTCCTAGAACTTGAAGGCTGAACACAC
AGCCTGGCATGTCACAGAAGCCAGGGCTGGGCACTTCCTAGCTGGGTGAGTCTCTCTCAGTTCACATAGC
AGGGAAAAGGACTACGTCACCCTGGCTACACATAGCCTCTGGGCTCAGGTCCCCAACAGAAGCTACTGGC
CTGCTGTGTTCTCTTCCCCAACTCCTGCTGGAGAGAATACATGGCCTCAGCAGAGCTGGAATCTGGGTGT
TGGTGGTCCAACCAGCAAGGATGGGGAGCAACATGGGTCTCCCAGGACAGGTGGGGGCAGTCCCACCCAA
GACAGAGAGTTGGGGCAGACTTCCTTAGGGAGGTGGCCACTGAGCTGGGTCTTGGGAGAGGAGAACAGGC
CTAGTAGCTAGACAGAGATGGACATGTTCTAGGCAGCAGAGGAGAGGCGGGGACACAGTAAGCCCCCAGC
CTCCAAACTCAGGCACCCCTGGATGAGGGCCAAGCTCCAGCACCTCTGCCTGGCTGGCTCAGTACCCCTC
CCTTCTGGAGGCGGGACCCAGCCGTTTCCAGCCCACTCCCCTGGGGCAGTATAGGATACCCAGCTTAGCC
ATCTCCCATGGCTGGCCTGGGCTGACATGTGCTCTCCCAACCTTCACCACGGAGCCATGGCAGCTGAGTA
TGTTTGCACACTCTGAGGCATACGGGGGCTGCCCTGGAGGGCCAGAACTGGTCACAAAAAAGCAGGGCCT
TCTCTGGAAGATTGTCTCTTATGCCTCTCCAAGGCCTTGGCACCTGATCCAGTGCTGGGCACACAGCCTG
GGCACGCTGCACAGCCCTGTAAGTCGGGTCAGTCCAGCCTCCCACACCTCAGCAAAGGACTGCCTGGTGG
CACCGTCTCTGCCACTGGGGCCTCCATCCGCCCAGGACAGGAGGTCTGAGCTCACACTGACTATTAGCAC
AGGGTGCCTGTGGGTGGGCCCAGAGCCCCTGGGGAGCTCCAACCCACATTGCAGGGCAATGGGGCAGCGG
GGAGCACAGGTGTCCCCAAGTTAGTTCCAACTGACTTGTCCCTTCCACCACACAATGACTTCCTTTGCCA
AGTTGCTGAGCTCTTGCAAACTCTTCCAACTCCAAATACCTGGCAGCTCCTGCAGCTAAAATAACAGCGT
GAAGTTGCTCAATTTGAGAATCCATTTCCTGTGCTTACAACAGATGCAGGGCTTGTTCAATGTATCATAG
GCCGTGGAAATATCGGAAAGAACTGACCAGGTCAAAGCAACACCAAACTGATGTCATTGAGTGGAGTTTG
CCTTTTTAAGAGACATAAAAATATAAACACCAGATTCCTTAATTTATAGAAATCTAAACCTAAATATGAT
TCCATATATCTGATACTTATGTACAGAGTATTTCTTTGTGGTAGCTAGTTCTGTTAAGAGAGGGGAGGGG
AAGGAAACAAAATAGGGAAACTGAGATGAGCAGAAAGGAAGGGAAGGAAGAGGGCACCTAGATAAGGGGC
TGGAAAGCTGCCAGGCTCTAGGAGAGTGCCGTGGAGGGGAGAGGGGAGGAGGAGGAGGAAGAGGGGACTG
GAAGAGCCGTGGACTCCCATAGAGGTCTGCAGGGGTGGCTAGTCCTTTATTTCAAGTCTTCCCAGTCATT
CAACACACCTTTGGCAAGAACCCTCTGGGTGAGGCACCACAACCAGGCACTAAGACGTGGCAGGAAATGA
AACAGGCTAGCCCCTTCACCACAGAGCCCACAGGCCAGCAAGGAGTTCGGACACTACACAAAAATGATCA
CATGATGAATTACAGCAGTGGAACGTTCCCTGAAGGACAGCCAGGGCTAGGAGAGAAGCTGCCAGGGAAC
TCAGCCTCAGCTGAGGGGTCCGGGTGGGCTGCTTTGCCACGGGGACATTACCATGAAGCCTTATCCAGGT
TTGGGGGAAGTGGTGGGGAGGGAGGAGAGGGTAGGAGGAACCTGAGGCATGAAGGGTTGTGGCCTTGGAG
GATCTGAGAGAGTGCTTGATACTGGCGAGGCAGGAAGAGACAGGCTTCTCCACGGGTCTCCAGCTTGAGG
GCTCTGCCGTGAGCCCAGGAGTAGCCAGGTGGACGTTTTCAAAGGAGCCCTGGGGCTGCTGTGTGGGGTG
CTGGGTGGTGCTCCACAATCAAAGGTGAATCTCAGGGGGATCTGGGCTTCACTGTGCAGAAAAGACTCAC
AGCCAGTTGTGAGATGGAACAGGACCAGGGGCCGTGAGCAGTGAGGAGCTGAGGTAGCTTCTGCCCACTG
AGCTGGTTCAGAAGTGAGGGGAGAGGAGGGTTTTCACCAAGAGGGGATAGCAACTCTATTGATCTTGGTC
ACTTTCCTTTGGAGGTTACCATGGGACAGATGTGGCATGCAGTGGCTGAAGGCAGTAGAGAAAGCATGTA
GACAAATAAAATAGCATGAAAGCACCAAGAGTCACACAATTTCAGTGTAACAGAAGGGGAAGCAATGGCA
TTGGAGTTAAGCAGTGCTCAGAGAGTGGGATGAGGGCCAGTCCTCAAAGAGTGGGGGTCGCAAAGAGGGG
AGAGTACTCAGAGGAAGGCGTGGAGGCCAGCAGCCTAGGTCCTCCAAGGTCTGAGAGGCCTCGTTATTAG
GAAGTCACTGGAGGGTGGGGGCAGAGGGAAGTCACTGCAGTGCCAGAGAGCTGACCGGCAGAGGGGAAAT
GGAGGCAGTGGGGGGATGTGTGGGAGTGTAGGGCAGGTGCGGAGTGGGCAGTATGTGACAGGGTTGGGAC
AGCTTTTGAGCTGGGACAGCCACGAAGAAGGTAGAGGGGCTGTCTAAGCAGCCTCCCCATCATGCTGGCC
CATGTGGTCTTCTCCCATTCCTGAGGTGGGAGTTGGCATGGAATGCAGATTCAATGAGAAGTCTATTAAG
GCCTTTGTCCTAGAGCCCAGGCACAGTGTTCTGCCTCCCCTACCAAGAGTCCAAGCTAGGGGAAAGTTCC
TAGGGACATGGATCAGACAGCCTCTCTCCTTCCTTTGCACTCAACTGTGAACGGGGCTGGGGAGGTTGCT
GGGGCTGGGAGCAGATGACCAGCTTCTGAGAGAATTAAGACCAGGCTCTAAGAGAAGCCACGGTCCCTCC
CTGTGGCCCTGGAAGCCAGGAGGGACCAGGCAGGTGGGTCCTCATGGGCTACAAAGACTGGAGAACTGAC
CACCTGTCCCTCAAAATCAATGACCAGGACAGACGAAGATGAAACTTCAGAGCAAGGGGAGAGATGGGGG
GTGGCAGTGTGGGGGTTGCAAAAGACCCTCCTGTGCACACACTGACTCTTCAGGACCCATTCCTGAAACA
TGGCTAAGATACACCTATGTGAGAAGCATGGGGTGGGGGAGGTGGGGATCAGGGAACTGCCCCACCCCAA
CCTCCTATGCACAGTCTCAGGGAGTGAAGCCATTCTCACTAGTTTTAAGGACAATGCGTTGACATGACAG
CATTGACACTTAGACAGATGCTACAGGGAGCAAGGTGCAAGCTAGACCTGAGACTTGGGGGAGATGGTGC
CCCTCCTGACCCATGACCCTCTCTGGACACAGGCAACCTTCATCTAATGTGAATCAATTGGGCAGGATGC
ACACATACAGCTTGGGATAAATTTTTCTAGAGGCCCCTTAGCTTTCATTTCTCTCAAAGAAAACCACCTG
AAGGGAGCAGGGAACCCTTCCAGCGGTGTGCTGCCGCTGGCTGATGCCATGCTCAGTGAAGTCATATTGG
TTGCTTGAAATGGACTGTGGTGAAAGTATTAACACCGTGGAAATAGACAAGCTCTATAAGTCAGGACGTT
TTTCTCCCAGGGAGACAGTTGTTAAACATTTACCAGCACATCAGCACGTGCTGGACCCATCTCTGCTGAC
CCTGACTTTCTCTCCTCAGTGAATGGGAAGGCCACGGTCACCATTGAGAGCTGCGCCCCGACGCTGTTCA
TGTTCATGCTCAATGGCAAGCAAGAGCATGGGCTGCTGAGCCTAAGGAAGAATGGGATGAAGTTGGAGGT
GTACCCTCCAACCATGGGCACTCACAAGCTGCAGATCTTTGCCAAGGGCAACTCCGACATCTACAGCTCA
GTGCTGGAGTACACGCTCAAGTGCAATTATGTGGACATGGGTGTCCAGCTGCCTGCTGAGCTTCACCAGC
CCGTGGGCCCCAGCTGGTTCTCGGAGCAGATGGGCATCATGAAGCCCTCCCACCCTGACCCTATCATCCA
CACCAGCGACGGGCGCTGCTCCATCAGCTTCAGCGTGGAGGAGGGCATTAATGTCCTGGCTTCCCTCCAC
GGGGATGATGGCCCCATCACTGAGGAGACACAGCGGCGCTACATCTTCCAGCTGCACCGGGAGAAGCAGA
CCGAGCTGAAAGTCCAGCTGCCCCATGCAGGCAAGTTTGCCCTCAAGATCTTTGTCAAGAAGAGGCAGGA
ACCAGGAAACTACATCTTCGTCTTTAATTACCTTGTATGCTGTGCCAACACCAAGGTGAACTGGCCCATG
TTCCCTGAGAGCTTTGGCAACTGGGGACAGGACAATGAGCTGCTGGAACCTCTGTCAGGTGTGCTTCCTG
CCAACCGGAATGTCCCATTCAAATTGAAGCTGCATGGTATTGCCAAAGTCCTGGTGAAGGGGCAGGACAC
ATGGCCCCTGACCCTGAACCACGAGGGCTACTGGGAGGGAAGCTGCAGCACAGCTGGCTGCCAGGAAGTC
TATGTCATGGTGCTGGAGAATGCCAACCACAATTTCTACTCCTACATCCTGAAATACAAAGTGAATGCCC
AGTGAGGGCTGTGCCCCGGTCTTACCCTCCCAAAGGGCCAAGGCCAGACCTCCCCAGGGAGGGCTCGAAG
GAAGTGCAGGGAGCCCTGGACACCACTGAGTCTGCATGAAATCCCTTCTAGGCCTCTGCCTCAGTGTCCC
ACCTCTGCCACAGGAGCTGTGATCTTTGTGTTCTGAAGGCTTCACTCAGGTGTGGCTGTGACTGAGGGGG
CAGAGGCAGTGGCTCTTTAGAAGGAAAAGGTGCTATGTAAATCTAAGGTATTGTAAACTGTACACCTGCA
TCCAGATGCCCCTTCTCGTATTAGTGCTGCTGCTGTTGTTGTTGCTGTAGGGTTGATGAGGGAAAGCAGG
GATATTGCCCAGGTTGACAGCTTTTCACATGATGCTTAGAGAATTCTCTTCCATGCCCAAGGGGACATGA
TTTGCAAGGCTGGGTTGTCCTTGAATCACAGAGCCCCTTTCAAGCAATATCACCGTGCCTCTCGACAGCT
CCGAGCCCCCAGGGAATCTTGGACTATCTTCTCCTCCCAGACCTGATCATCTTCTGCATCCTGCTGCCTC
TTCATTAGCTACTTCTTGTTCTTCAAGAGCCCAAGAAAAAGGGTGTGGTGAGGAAGCCAGCTGACCCACA
ACTGGCTGGAAGCTGATCGTCTCCAACATCCCTAAGTATAGTCAGGAATGTGACAACCAGAAAGTTGTGA
GGAGACTGCAAAGCAAAGAGCAGCCCATGCCCGCTGTGGCTGTGCAGGTGGGAGGGACACTGTCGGCAGA
GCTGCAGCCTTGGTCTGAGGGTGTAAGACCTCCAGCTCTGCTCTGAGAGGAGTACACAGGGCAGGTAGTT
CAGTCTAAGGCCTGGAGTGCCCTCCAGGCCCTCACAGAGCCTGGGCCTCAGGCCCCTGCCCCACCTCCAC
AGAGAGCCTCCAGAGGCTGGCCAGGCCCTTGGGGAGTCTTGTGGATAGCCCTGAATAAGGACCTGGGCGC
AGATCAGGTGAGTAGTGCAGTGCGGCAATGCCGAGGAGACTCACACTTTAACAGGGCTCTGCCTGCCCCC
AGTGTCTCTACCAGCAAAGCCAGGGCTGGGAAGAATTCCAGCTGCACGAGAGCAGCTTTTGTGCCTGTCT
CGGAGAAAACATCCTATCCCGTCTCCTTTCCTCCCTTCTCTGAGAAGACAGCCCCCTCCAGTCTCACCTT
GCATGGGGACAGCTCAGGGCTCCTTGCTTGCTCAGGCACAGACTGAGAGGCCTCAGCTGCCCAGCTGGCC
CTCAGCAGCGCCTCAGGCTGCCGTTGCCATGGCAACGGAGGAAAGGCTGACTTCAGCGAGGGCAGATAAG
AGGGAAAGGCGAGAGAGGCCCCACATGGCCACTGCAGAACCATCCTGGCACACAGACATGCTCCCTGCTG
AACTCCCGGCTTCTCCAGCTCCGTGGCCCCTGGCTGTCTCAGTGCCCCCCAACCCCTGAGAGCCAGCACT
GCCTGGCACCTCTCCCCACAGCCCCTCCACAGGCCTCCAGGCTCAACCACAGCCAGGGCCAGCTGGGACC
CTCATAGCTTCCTCCGTGGCACTCACCCTCACATCCCTGTATCCAACAAATGCTCACAGAAGTACCAGAT
GCTTGGGCTACAGAGGTGAACCAAGGGGGCCCGGCCCCTGCCATCCAGAGTACAGGGTGAGGAAACTGCC
AGGCAGCAGCAGCCTTCCCTGCCCAGGCCTGACTGAAGATCCAGGCTCCAGACACTTCTGTTCATTATCC
CCCGGGATCCCTCCCCTAAAAGTCCCCACTGCCACCCAGCACGGTGGCCTAGCCCAGAACCAGCTCACAA
GGGTGCCCATCATCCAACAGGCCGGCGGCCTCGAGCAAAAGGGGTGTCTCCCCTGGGGGGCTGAACTGTC
CTGGGCAGGTCCTGCGCCCTCCGCAGTCTCTGCCACCGAGGCCACCCAGCCAGCCTCGTGTCACTCACAC
CTGAAAAATGCACAAAGGCCCCGGAGGCATAACAGCTATGCACCACCACAGGGAAACAAAGGGAGTTTTA
CACCCTGAAAACAGCCGTCTTGCACACTTCTGATTTTATGGGCTTAAAGAAATGTGTTTGTTTCATCAAA
ACAATAAGTGCCTTCGATGACGCATGTCACTCACTGGACCCCGCGCTGGCTGCCTCGCTTAGTCCTGCTG
GAGACCCCCATACCTTTTCGATATGCAATATAATTAATCAATACATCCTGGAGCATTGCAGAGAATGCCA
CCCAGCAAAATAGGAGCTGTCCCCAGCACCAGAGAGGGAGGCCCCGCCCCTCAAAGCACCGCAGGGCATC
AGCAAGCCCCTCCCTCTCGTTTAACTAGGCACCCCCTTTGTAAAGACAGCTGTAGGCGCAGTCAGAAAAT
CACCTGCATTTCTTTCCACTGAAGTAAAAATACATGAAATGAAATCTAATCTTCATGAATTTTTACACGC
ATGCTGCTCAGGGTAGACATTCGGAGTTGCATTTTGAAGGCTTTTCTTTAAATCCCCATCTTTGTTTCCC
GGGCGACGAGTCCCAGTCGCCGGCGGAATGGCACGGGCGCCCCCAGGTGTCCGAAGTGCGGAGCTGCCGG
CGCTGCACACCGCGAGGCTGATCTATCACCGGGCTTTTTCGCAGAACTTGAAAATGCTGTGGAAATGTTT
ATGTTAAAATCACTAGGTGCTTTCTCGTGGCTTCTCAGCGCTACCTTATGTGTACACGGAGGACATCTTT
GTCACGCACAAGCTGTGAGGGCATCCTGGGGGACAACCTGGTGTGTTGAAAAGACAAGGGATGTGATGGG
ACTGTTTTGCCTGGAAATAACTTCTTGAAACAGAAGCACTTGACATAGGACTGGATGGAACAGCTGTGAC
TACACTCATTACTATACCATTGAAGGATTGGAACCGATCAAACTCAGAAAGTATCTGCTTGCAATTCTCA
GGATGGCAGCCCCCAGGCCACAGGGCAGAAGGGAACCGAGGGTGGAGGAGTGGTGGGCAGAGGCACTACA
ACCACAACTTCAGTACACTTCTTTCCAGCTTGTTTCAGAAGTGCCATAGCTCAGCTGGACTCCATTTTCA
ATCTGATTATGATCATTGTGAGTTCCCCATATGTTACATTGTCCTACCAGGACAGCCTCCTCACTGCCTG
CTAAGATTAAAAGTAAATAAATAGACTAACTGAAGCATGAAGGATTTCGGCTAGACATAAAGAACTATTT
CCTGTTGTTAAGCAGGAGGGACAAGTTGCCAAGAAAGTTTGTGAAATCCTCTTCTCAAAGGACTTGAAAA
GGAGAAAAGTTTTCCCAGTGGTCTGAGGTTTTAGATGCCATGTTCACTAGAAGGAGTGGGTTTTCTCCTC
TCCAAACCAATTAATTCCTGAGATATCCTGGTCTGCCTGCAGCAAACAGAGGGTTAACAGCATCACTACA
TCATTTGCTATTGGCAAAATTTTCGAAGATGGTGTGCCTCTATTGCCCGCCTCAGTTTATCTTAGGTCTA
TAATTTCTTTTCATTTGGAGTAAGTAGTCGGCAATTGAAAGAGGTGATTACTTCTCATAATGTGTGCATT
GAGTTGTGTGGGCCGGTGGCTGTTGAGTATGCTTTCTCCATGTTGTTGCTGGTGTTGTTTTTCAATAAAA
TTTATATTCATTTTTATCCAACAATGTAATTATCGATTCATTCTTATCATGTATCTCACAAGGTGGGAAA
TGGAGAGAAACAGGAAAAATGCTTTCATGTTCAAAGGAAGCCCTAGAAAAAAATCAATGTCCCAGCCAGT
TGAATGCGACGGTGAGATGCGAATGGAATGTCAGGCAAATAGCTATTTCACATTAAAGAAAACATAATCA
GGTTAGCTCCAGCCACTCTATGGTTCACATGCCTTTGAAGAACACGAAGGCACTGTTTCTTTCATTGAAT
CTTGTTTAACTTGAAAATTTTAAAGTCAATCCTAATTTATTTTTACAGTGGGAGAAAAAAAATTACCAAA
AATGCCTATCAGCCTACTTTTCTTTTGAATGTATATAAATATGTACTTCTTGATATTCATATACATGCAG
AAACTTTCCAGCCGGTCACCTAAGCAGATTATTCATAAACTAGACATTTAGAGAAATACAGAAAATGAAA
AATCCAGACATTTCTGAGAAGCTGCTGTAACCATGGAGACCATTCTCGTAGCTGTAGTGTGAAGGACAAA
AAGAACTTGTGTTCCCAAATCTGGCTTCGTCATGTCTCTCGCTGCTAGATCCACAAAAACGAGGCCCAGC
ATGCTTCTCCTTAGGAAGGAGACCCGGCAGCATATTAAGAGGAAGTCAGAAGAAACAGTCTTCCACACAA
AAGAAGGAGTAGCTCTTGAGTTATTAGAACAGTCTGAAAATGCCATCCCCAAAGCTGGAGTGGATGACCT
CCAATGTCCCTTCCAGTCCGGAGAGTTTGTGATGCCGTGTCTCTGTGAACAGATGCTGGCAGGCAGTGGG
CCTGGGGTTGTCAGTTACAGAACATCTGAAGGGCCCTGCTGTGGAACATCTGAAGGGCCCTGCTGTGTTA
AGAGCCTGCCCCTGGAGCCCCTGGCAGACTGCACTGCTGGCTCCTCTCTTGCCTGAGGGCAAGGGGCTGG
AGAAAGCCCAGCAGAGGGACCAAAGGGGAAGAACAGCCTCTGACAGGATGCTGGTTCTGTCTGATTTTGC
AAGCAGAACTAAAGGAAACTGAAAGTTAACGGGGATGTCCTCTTTGGGGTGTCCCACAGGCATCTCCGAG
TCAAAAGATGCTAAACTGGAGTTACTTCTCTCCCTCAAATTACTCCCAACTCCTCCTGGGTCCCCAGTTC
AGTAAATGCATCCCTATCCAACCAGATAACTAAGTTAGACTCATGGCAGTCACTGGTGATGAAACCCAGT
CCCTTCTGGATGTGTCTCACCTCCCACCTGCTCTCCCAACTCCACCTTCTCTCCTTTCCCACGACCTGCT
ACAAACCAGGTCCTCATGATCTCAGCTATACTGTTTGTGTCTTATACTTTCCATGGGGAGATGAAGCCTC
CCGGCCCTATTACTGTCCACAATGGCCTCCCATTCATATATTCTTATCACACTAGGACGATAGGGTTGTT
AAGACAAGGTTTCCAAGAAATCCTTTCAGAACAATCTCACCATCTCTGGAAAGACATAATTGGTATTAGG
CACTAGCGGGAAAGGCAGGTCTTCAACAAAAAACTGGTTTTTATAGGGAATCGGAAAGAAGCACTGTCTC
CTTTTTCTCTAAAGAGACAAGGTGCCAAGGCCACCCAGTCAGCCTCGTGTCACTCATGCCCGAAAAATGC
ACAAAGGCCCCAGAGGCATAATAGTTATGCACCACCACAGGGAAACGAGGGGTGTTTTACACCCTGAAAA
CAGCCTTTTTGCACACCTTTGATTTTACGGGCTGAAAGAAATGTATTGTGTTTCATCAAAATAATAAGCA
CCTTCGATGATGCATGTCACTCGCTGGACCCCACACTGGCTGCCTCGGCCTACTTCACTACTGGGTGAGG
GTATGCAGGGTGGCTCTTCGTACCACAGCCAGGCGGGGGACTCAATGGGGTGCAGAGGTGGAGCCCTACC
TGCAGGTAAACAAGCTCCATCTTGAAGCCAGACTCTCCACATCCATGGCTTCCAGCTTATTTCCCTCCTG
TCCTCCTGCACCAACAGCAGCACAGATCATATGTTCAGGTGGACCAGTTCTATTCCCAGGAGAACAGATG
CTGAGACACAGTAGGAATGTAAACAGTGTGCTGGAGGCAATACCTGTGGAAGTTAAAAGGGAGGGGGGAA
GCAGGATGAGTCAGAGAGAAGCTCAGACAATGGTGCAAGTCTGACCAAGTCCCAGCCAACCCAACAGGAA
GTTCCAAAACAAAGATTTGTATTAGAAGAGTCTGGAGCTGTACAGAAATGGCCAGGCCCTAGAGAAACCA
TCATGCTCAGCCACTTGCTGGGGCTGCCTGGCAAGAGCTTGGCCTCAGTTCAAAAGCTCAGGCAGACCTT
GAAGGTGCTAACACCTGGAGTCTCTCCGCGACCTGCACTCCTTGCAGAGAATCAGCAACTTCTTCCAGAA
GGGAGACCTGTGCAGCACACACAAGTTAACTTGCCTATGCTATGGAGGGTTCAGGAAAGACTGGGTGAGG
GTTCTGTGTGTGCAGTCAGCATACCCAAAGCCATCTGGGATCCAGCAATCACTGAAAAGTCTTCACAAGA
TGGTTTGTGGAAGGGACAGCGGCCTCTACATAGGAGAAATATGTACTCCAAGGGCCCACTTTCAAGGTGT
TGTGGACTAATTTGATGAAAGGCAGCTTCTTTCTTATTTCTCCTTCTCTCCCTCTTGCCCCTGCCATCCA
GGGAAGCCTCAACACAACAGCCAAGAGGATCGCTCCATGCTGCACTCTTACTTAAAACCGACCAATGGTT
CCCCATTGCTCAGGGATGAGACTAGACCCCCACTCTGGCCTCACACTGCTTTCTCCACTTTGTCTCCCAT
CACTCTCCCTGAGAGCCCCTTAGAATCATCAACCATGTGACACCAGGCAGTCCATGTACCTCCCACCAGC
GTGCTTTATACATGCTGTCCTTTCTAGCCAGGACGTCTTTTCCTGCCTGAGGAAGCCCTGCCTGGCCTTT
GTGGATCAGGCCAGCCCACCACTCTGTCAGACCTTCCTAGTCCTCCCACTGCCCAAGCCATGGGGAATTA
TCTGCCTATCTTCCATGTTCATGTCACACTTTGTTGCTCATCTGTTAACCCAGAACCTTCTACAAGATTC
TGGGATTCAGGAAGTACTTGGAGGTTCTGCCCCTAGGATGTGAATTCCTTGAATATATATGCTTTGGTTC
TGCTCCTCGGCACACAGTAGGTGTTTGGTAAACGCTTGGCAAGTGTTTCACTGGGAAAACTAAGACAGAC
TAGGAAGACAGGGCCAGGAAAGTACTACTGCCTAGCAGTCCATAACCATCCACTGAGCATTTGTCAAAGC
ATGTTCCCATTTCACCAGGTCTAGCCCTTGTGTTTTTGCCCAACTCTGTATGCCACATACTGGACATTGA
GGAGGGGAACCCAAGATTCTCTCAGAGGAGGGCCACATCCTGGGGAGGGCTGGGAATGTGGGGTGAAGTA
ACCACCCTGGAGGAGGCTGAGGATGCAGCTAACAGTGGTCTTCAAAGGAAGAGGCCCTTTACCAAGTCCA
GCATCTAAAAGAGGACACATACTGACTACTTGGAAGATAGAACAAGTCCTGAAAGTAGGACTTGGTCTTG
ACTAGAGAAGGATGGCCACCCTGCCTGTAAAGGTGGGTAGCAGAGGGTTGTCAGGCATTCTTTGGCTGTG
AACAGTTTTAATGGAATTTTCTTTCCTTTATCTAGATTTGTTCCCGCTCCAAATTTTCCCCATGGCCCTG
TTTGAAGATTATATAACCTAAGCTTGATGCTTCTATTTAACCCAGAGTGACATAAAAATTTAGCTGAGGG
CACCTGCTGATCCCATTCCCACCCACCCACCCAGAGACCAACAAACAAACCTTTCCACGGGAAAGCTTCT
AAAGCCCCAGAAGAACCTGTTTTCTCGGGAAAGAGCTGCAAAATGGCAAATATAGCTATTTAGGATCTAA
AGGCATTAACATCTTCTACAGCAAAGCCTGAGTATGTGGGGCTGTCTTGAAGACCGTTGATATATTGGAA
AGTAATGCTCCAGGGAACACTCAAACTCATGGTTTCTTTGAGCACAGTTGTCCTCAAGGCTGATCCTCTG
AAGAGGAGCCCCTGCAGGATGAAGTGTAAAAAAGACAAAGCACCACGAAGTGGTATCCAGTGGTATCCAG
TGTATCCAGTGGCAAAGCCAAGCCATGAAGGATTGAGAATGAGAAGGCCACCTAGACAGGGCCCACCAAG
AGATTCCCTCTCTCCAATTAAACCAGCACTCTAGATGTTTAGTCATCATTGCCAGGGGTACTGGGTTTAG
ACAGATTCTGAGGCCACAACTGGGTTCACTAGGAAAGAGTGTCATGACTAGCTAGTGATCCATAGACAAT
GGATTGAAGGGTGAGGTGTGCCATGTATTTGCCAATCCCAGCTTAAAGGTAGCTAAATCAGGGCTCAGAC
CAAAATAATGAAGGAGACTGTTTAGTCCGTTCTCACACTGCTAATAAAGACATACCCGAGACTGGGTAAT
TTATAAAGGAAAAAGATTTAATTGACTCACAGTTCAGCATGGCTGGGGAGGCCTCAGGAAACTTAACAAT
CATGTCAGAAGGGGAAGAAAACACATACTTCTTCTCATGGCGGCAGCAAGGAAAAGTGCTGAGCAAAAGG
GGGAAACACCCCTTATAAAATCATCAGATCTTGTGAGAACTCACTCACTATCACGAGAACAGCATGAGAG
TAACCACCCCCATGATTAAATTACCTCCCACTTGGTCCCTCCCATGACACGTGGAGATTATGGAAACTAC
AATTCAAGATGACATTTGGTTGGGGACACAGCCAAACCATATCAGAGGCTATTCAGACCAGTGGGGAGCA
CTGGCTTCCCATTAAGCTAGCCCATGGACTGACAACCAGTCAGCTTTCAAACCAAAGGTCCCAGGATGTG
CTGGGGGCCTCCTGACCTCACCTGAGCTCCAGGAGGATTCCACGCAACATGCATGCCACCCAGCTCAGAG
GACACAAATGCACACAGGCATATCAACTCTAGGGAGAAGGAGCCTAGGCTGAGTGTGTTTGCTCTTTTAT
CTCCATGATTAAACAGTGCCTTGCATTTAGAATAAGTGATCAGTAAATTTTTGAAGTGAACATTGCCTTG
TCATTTCAAAAACAAAATGATAGATCTCTGGGTTGTTTACATGGCTGTTGACCAAAACAGCTGTTGAGTT
ACAACATCCTAAATAGTCATTTCCTTCTTTTGCTCAACTAGGAAAGCATATGCACAGCAGTGTACTCCTT
TGCCCCTGTGGGAAATGTATATTTACAAGAGGAGAAAAATTGCCACCTGGCTGGGAGTGCTCATGGTGTT
TGCCTCTGGGAAACAATCAGTGCATGGGCAACCAATCAATGGGAGGAGTGCCGTTCTTCTCGGGGGAATC
CGCCCGCCCTGAGCATGTGCCGAGATTGCTTTGTTTCTATCAGGGGGAATGGCTGACTGCCTCTGTGACA
CAACGAATCAGCCATCATTTCGCCCTGGCTGTGCAGGGACCAAGGCAGTGACGTCCCCCTTTATGGTGGC
AGGGGCAGCTATGGAGAGGCTGGTGGCATGAGGGCAGAGGGGCAGCCACAACTGTGAGTACACAAGAACA
GCTGGGCTGAATGACTGACGAAGTGCAGATATTAGGAGTGGGGAGCCCCTGGCAGCTCCCATCTGGGTAA
AGGATGGTGGTGAGGAAAAATGAGGAAGCGTTTATTTAGAACAAAATGACGGTGGGATTACAGATTTCCT
AGTGAACACTTTGCTCATATTTATATTTGTATTTGAATGTATGTGAACACCAAGGAGATCTGGAAATTAA
CATCCACCATTTTATTCTATTTAAAAAGTTAAATGAAGCCAAGATACTGGCAGCACTTGAAGGCTGTCCC
CTCCTCCTATGCCCTTTGGGATAGCCCCACCCTAGGGACTGGGCAAGTGTGGGTACCATGAACCTTTTGG
CATTCATTTCATGAGTATCAGATCCCCTTCTCCATCTCCCAGGAGCAACACAGTGGAGAGAACACGTTAA
CCTGATTGTGGGAGGGCATCCACTCTGGCAGTGAAAGCTTCACTTGACATTTTTAAAGCTCAACAAGAGT
GTTAAAGTCTATCTTTAAAAGAAGTGAATGGGCTTCCTCTGGAAAGATGGCCTCTTAGCCACTGGTCCCC
TGTTAGACTGAAATCTGTCTTGTGTGGTCAGGAGCCATTAATGTGTGATTGAAGCCTCGAGTTTTCCTCT
ATACACATTGATTTCACTTGGAGAAGCCCTGTGGACCCCTCCTCCTCCTCAGCGTTGGCAGTCATGAGCC
TGGGGAAGCCTGTGGCTCAGCCCCTGTTTGACAGCAGGTAGGAGAGTGTGCCAGCTAAGAGCACACACCT
GGAGCTGACCGCCTGATTCCTAACCCCAGTTCTGCCAGCTCCCAGCTGATTTGTGCAAGTTACTGAACCT
CCCTGTTTTTGTTTCCTCATCAGTAAAACAGGGATACTCATAGCACCTACTGTGTACACTTCTTATGGGG
ATTAAATGAGCTAACATATGGTACAATGACTAGAAAAGTCCCGGGCACATAGTAAGTACTCAGTAATTAT
TGGCTACCATCACGATGCATCAGTCATCATCAGCATAATGAGGATGACACTGATGTATTGTTCCCTAAGG
AATCCAAAAGTCTCAAGCCCCTCATAAAATTGTGGCAATCACTGATTTCAATGCAGAAATCCCATATCCA
AGACGCAATCACCCAGTCTTTCCTAGAATCCTTCCAGGGACCAGGAGCTCTCCACTCCATGAGGCAGCTG
TCCATTACTGGACACCTATGTTAAGGCCTTACCTATACAAAACTGCCTTAAATTAGGGCAGCTGAGCCAA
TCCACCCACTGGGCTCAGTGAGTTGTCTATTATATGATGGGACAACCATGTAGTGGGAGCCCGGAATCAC
CAGTACAAAGCCTGGCTCTAGTCTGAGGGTTCCGGGGACTAGGTAGGAGCAACTGCAATATACTCAGACA
CGGAAGGGAGCCAAAAGAGCAGATGAAAAGAAAAAGAAAACCTTCCACTCGAGAGAGCTGTAAATCAACA
TACGAAACCACATGAAGAAAATTAATAGTTAAAAAAATTAATGAATTGTTTTCAAAAATACAATCTGCAT
ACTGAATGGTGAGACCACCTCAAACCCTGATGTTATTTAACTCCCCAAACCTGGCTGGCTGACTTTTGTC
CTCAAAGCAAAAACTGGAGGGTATCACTGAGAAAACAGATCAACCTAAGAAAAAAGGCCTACAGGTACCA
TCTGTAACCACCACCCCAGTGAAAAGACCAGCACCCCATTTGCCCATCTTTGGGAAGCCAGTCAGTCAGT
TCCTCCATACACACACACACACACACACACACACACACACACACACACACACACCAGTTTGCAGATGGCA
TTTGTGGTTGTTTGAAAAAATGTCCATGAATTCTTTGGTGCTCCTACATTCAAAAGGAAGAGCCTAATTC
CCATCCTTTTATGTGCAGGCCATACTTAGTGACTCTCTTCTGACAAATAGAATGTCGCAGAAATGATGGT
GTGTGACACTCAAAACTAGGATATAAAAGGCATTATAGCCTCCTCCTTATTCTCTCCAGATCACTTGCTC
TCAGCAAAGCCAGCTGCTAGTTGTAAAGATGCTCGAGCAGCCTCTGGAGAGGCCCACATTTTGAGGAACT
GAGTGAGTCCTCCTGC

An exemplary Cep63 human DNA sequence is provided below.

gi|224589815: 134191182-134307246 Homo sapiens chromosome 3, GRCh37.p13
Primary Assembly
(SEQ ID NO: 303)
GGTCCCAGCTCTGTAGTTGTGTTTCTCACACCTGCAGCACTCAAGAGGCTCGATTCTGCAACAGCTGCAT
TGCTGTGACCAGGGTGACAGAGTGCAAGTTCATATAGTAGTTGGCTAAGAGCCAGATGAAATTTAGGCAG
GGACATGCCCTGAGATTGGGTGATAACCTCACCTCAAGTTCTGTACCTCTCCTTCCTCCCATGGTCAGTG
AGGCTAGTTTTCAGGAGGCAAAAAAGACAGAGGGTTCAGTTGAGCTGAGGAACCGATTTCAGCAACTGCC
TAGGAGATCATTCGAGGACCTGAACCCCTGTCAGGACTCTGGCAAGCCCGGGGGTCTGACCCAGCATTCT
CCCTGTCGCTCCTCCTGGCCCTGGTTGCTTTTCTGTCACCCCAAAGGCCAACACCCACAGTCCCTTGAAT
TCTCACTTCTAAGGTGAGGGCATTTGCTGGGTTTAGTCACTGTCCAGCATGGAACCCTGATGGATACCAT
AGATTCAAGGATCAGCTACCCAATTTATGGGGCCAAGTACCAAATGAAAATGTGGGTTCCTTGTTAAAAA
ATTATTAACACTTTCAAGACAGCAAAAGCGGAGCATTATACCAAATGCAGGCCCTTTTGAGTGCAGGCCC
AGGGCAACTGCCTTGCTTGCACGCCTGAGCAGCTAGCTGGTCCAGGCTGGTTCCGGTGCAGAGGCCAGCA
CCATCCATCATCTGGGCCAGGAAGGCCACAAGGTATGAAGTCCCTTTAGGAGACCACCCAGCTGCCTTTT
CAGGCCTGCCACTACCCAGTCTTTCCTCGAGAGCTCACTGATGGCGTATGGCACAGTGAGAGTGAGAAAC
TGGTGAACAAATATAACTGTGGGTAAAGCGGGGCCAAATCAAGAAAGGAAGCTTTGAAAAACAGTCTTTT
CATGGAATTAACCTAAATGCCCACCAATGGCAGACTGAATAAAGAAAATGTGGTACATATACACCATGGA
ATGCTATGTAGCCATTAAAAAGGAGATCAGGTCCTTTGCAGGAATGTGGATGGAGCTGAAGGCCATTATC
CTTAGCAAACTAACGAAGAAACAGAAAGCCAACACTGCATGTTTTCATAAGTGGGGGCTAAATAATGAGA
ACCAATAGGCACAAACTGGGGAAAAGCAGACACTGCGGCCTACTTGAGGGAGGAGGGTGAAGGAGGGAGG
GGATCAGGAGAAATAACTACTGGGTACTAGGCTTAGTACCTGAGTGATGAAATGATCTGTATACCAAACT
CCTGTGACAACAGTGTCCCTGTATTAACAAATCTGTACATGAATAAGAGTTTTTGTTTTGTTTTTTTTTT
TTTTAAAAAGAAAAACAGTCTTTCTAGAGCCTAATGGATTATTCCTGGATGATGCCTAAATTATCCAAAC
GATTTCATGCAAAGATCTGGGAAACAGCTTAATGAAAGAGCCCCAGGCCTGACTATAAGTTGGGGACACA
GGTGGGCAAAATTCCTGGTTCCAGACCTCTTTTTCTTTAACCAAAAGTTCTGAACTGCCCTCAACTTTTG
GGTGCCCTCACTTTGCCCCCGACAAAGCATGAGTGGGGAGCCCACTAGGTGAGCTGCTCAGTCCAAGCCT
GGCTGGCCAGAATCCCAGGAAGGCTGTGCTTTCCTCACATAACCGCTAGCTAGCACTTTGGGCAGCCCAG
GTATGCAGAATCACTTTCAAGCGAAAGATCACACGTATTGAGAATCTTCAGCGTTCAGAGCTAACAATCA
GCCAGTGATTCAAACTTTCTAAAAGGAATTGAGCGCCCCCTGCTGGGTATTGTAGCATTGACATTAAACT
ATCCTCTCAAATGCATCAAAATCTGAAACTACAGAGATTTAATCTCGTATAAAATCCAGTGACAACAGGA
CTCATGGTGACAACATTATATATGAGTTTGATAAAAGGTTGAATCTTTAAATGTTTCATGACTCCCATGC
TCCTATTAAAAATGCTTCTAGTTCTTTATCTCAATTTTAGAAACAAGGTACACACTTTAAAGTTCTAAGT
ATTCTGGTAGGATTTTCTTCCCATGTCACAGAAAGTTTTACAGGAAAGGTAACATGGTCCTGGGAGAGGA
CATGGGTTTTGGGATCAGACAACCTAGGCTGGAATCCATTTACTATTTGAATGACTTGGAATAAGTCTTT
TTTTTTTTTTTTTGACGGAGTCTCACTCTGTCGTCCAGGCTGGAGTGCAGTGGCACCGTGTCAGCTCACT
ACAACCTCTGTTTCCTGGGTTCACATAATTCTCCTGCCTCAGCCTCCCAAATAGCTGGGATTACAGGCGC
CCACCACCATGCCCGGCTAGTTTTTTGTTTGTTTGTTTTTGTTTTTAATATATTCTTAGTAGAGACGGGG
TTTCATCATGTTGGCAAGGCTGGTCTCAAACTCCTGACCTCAGGTGATCTGCCCGCCTCAGCCGCCCAAA
ATGCTGGGATTACAGGCGTGAGCCACCGTGTCCAGCCTGGAATAAGTCTTTCAATGTTAACTTGGAACAA
ATCATTTAAGTCACCATCTCCTGATCAATAAGGACAATGCTATCCACCTAAAAGGTGAATGTCAGGGCTG
GAACTCATGTGTTTAAAGTGCATAGAAGTGCTTGATTAACCATCGTGTCATCGTTACGATACTATTACGA
TGTGAGTCTAAGTTGGGTTTCAAAGGATGAGCAGCCCCTGGACTCAGACCTTGTTTCCAGAGACTAGGGG
CTGTAACTTTAGTCCTGTGTCTAGCTCAGAGATCTAACCCCTGGGTCATGAGGGCAGTGCTCACACTGGG
CAAGTCTGGACAATTCCACCTCGAACAAATATTTCAAGTCCTTCTACTTTTACCTCTATCTTGCCAATCC
AAACTACCATCATTTCTCACTTGAGCCAAAGCAAAGCTCTTCTAATTTTTCTTTCCACTTCTATTCTTAT
CTCCAAAGTGATATATACATATATATATATATTTAAATCCAATCTGATCAGGCCACTCTCTCTAGTTAGA
ACCTTTCAATGACTTAGAAAAACTTTTTTTAGCCAGACACCTGCCTACCCACCTTGCTTATAATGCCAGT
CCTACCTCCTGGCTTTATGCAGTTAGCTCTCTCTGTAAGCAATCCTCATCTCCCAATTCTTTCCCTTATT
ACTACTACTCATCCTTTGGGTGTTGGGTCACAAGCTCAAGGAGGGTACAAGAGATAGCACATTTCTTTTT
TTCAAAGCAAATTAAGTCCATTTGGCCTCACTCTGCTGCACTGCAAGCAGAAATTAATTCTGTGGTCCCT
GGGGTGTCCTTTCCCCTTCTTAGGGTCTTGCAAGCATTTAGGACACTCATGAATGACCTGGAAAAATGGG
TAAAGACCTCTACTCTTCAGTCTATCTGGGTCGTTGCTATTATCTTCACTGCCAAGTCTGAGGGGGCCCT
TGTAGGAGGGGGTTCCAAGCTTGGAGGTCTTTGCCTTTTCTGGAAGACTCAGTGTTGAGGTGCAGGTTTC
CTCTGGAGCACCCAACAAACCATGAAACTCTGAAGTTCACCATCTCACTGTGGTACTGAGTGGAACACAG
CTTTCCACTACTCCTAAGAGGTTCCTGTCTGCAGACCCTCTTCTCTCCCACCCTCCCCTGCTTCCCAAAC
AAGGACTAGGGTTATTATTAAGCATGTCCGCTTCCTGATGTGCACGGGAAGCACACAGGACCTGGTTACT
TCCTTGGAGATTGGAAAAAAATTCAGAGAAAAAATTAGTATTTCAAATTATGTGCTATAAAAGCTGCTAA
TCACAAGGTCTCCCTTGGATTATTCAATATAAATCAGATCCCCTCCAGTCACATTCTCCTATACTGCAGC
AGTACTGTTGGTACCCATTCCCCCAGCTACTGTGTATTGGCTGTCAAAGGCTCACAGCTGTGAGAAGAGA
TTTTAGTTATTCAGTGTTTCCAGAGTTTCAACAAGGGATACGGACACAAGCAGCCGAGGCTGCTTCTTAC
AGAGTGTCCTCTGGAGAGGCGTGTCACCTGGCAATGCCTGGGAGGTTATGCTGTCACTGGGGGATTGTGG
GAGGGGCCAAGCCAATGACTGACCAATTCATACTCAGTACAAAGGCCTAACCCCTTGCCTCAAGGTGAGA
AAACTCTTGGCATCGTCATGGGCCAGAAATCCCACTGGATCAGACTGAAGCTAGACTTCAGCTCAAACCA
CATCTTGGCTCAGCTTCTTTCCCTGCCCTATCCTGTTTACCTTACAGGACTCTTCTGAAAGCACCTCAAT
AAATCACTTGCACAAGAAATCTCTGCCTCAGGCTTGGCTTCTGGGAACCCAACCTAAGTCACATCTTTTA
TTTTTCCTTCCTAGCATACGCCCCAAATTGAAATCATGTATTTGTATGGCTAATGGTTTAATGCCTGTCT
CCCCAACTAAAGGCTCCATGAAGGGAGAGGCCACACTTACTTTGTTCATTGCTATACTCCCAGTGCCAGG
CATGGTGCCTCCCACAGAGAAAAGCAATATGCAGGCTCTAAACTGGAAAGCTGTCAGGAACCAATAGGCC
ACATAGCCTCTTGGCCTCCCTCTCCAGATACTTTCCTTTTTGGGTGTTTTGGTACAGACAATTGAAGAAA
GCTACCCTGTAACAGCAACTTAGCATTTCTTGTGCCCATCATCCTCAAGCCAGTCCTAGACGGAGATTTC
GAAGCCCTGGTTGACAAAATCTGACTAGCCCAAATTAGGTCACCTAATTCCTGATACAATGAGCTACAGT
TCTGGCACCATAACACAAGTTACGAACTTGCCCAGAGGCTATTCCTGGGGGTGGCGGCAGTTCTCGGATG
AGTGTCATAGACACAAACCAACAGCTGTCTTACAGATATCATATTCTGGTTCAGCAGAAATCAAGAATAG
CTTTCATATTTTACTGGTCTACTAATATTCCAGGGAAAGGCTAGGCAGGATTCACATGAACATTTGTCAA
GTTCTTAGATGTTAGTGTCTCAGAGATTACATACCACAAGTAAGCTGTAAAATCCTCATTTAACAAGAAT
GTGAAAGAACTGTGGCGTTCTAGGTATTTTAATTTTTTGAAAAGCTTTTTCTCTTGTATAAGTTTTTTTA
ATATTGTAAGTCTCTTTTCTATTAGGTTGTCTTTCTTTGCTTACTGAAGGGACTGGTGCCTTGGGGTCTT
TATTCTGAAGATAAAAGATTTCTAGGCAGAACTACTGATGCAAAGAGAAGAAATCAGCCTGGATGTATCT
GGATTATAGGATAGCTGCTAAAATCATGTTTGTGATTGAAAACCTAAAATAAGCTAATTTGTTTTTTCAT
GTACCTCTTCCTCCTAAAAGCTAAGTAAACAAAAAGACCCAGCATAAAAAGGATGAGAAGAGATTTTAGT
TATTCAGTGTTTTCAGAGTTTCAACAAGGGATACAGATACAAGCAGCTTCTTACAGAGTTTACAATCTGG
GGAGAGAACATGAAAGACACTGTTTAACAGGCAAATAATTCCAGGAATAAATATACATGAATGTGTTTTT
CAAAATACAGGTTCTTATACAAATGTATAACTAAATACTGATTCCATAGTGGGGTGGTTGTAACTGAAAG
GGCTTTGAGAAAAGGCTTTGAATAAAACTAGTCATCCACCTAGCCAAAGATCCTTTCCAGCAGCACAAAA
GGAATTTGTAAGGAGAACAGAGATTAACTGTCAGATATCTTTCTAATCTGTAAATTTATCCAAAGTTTGA
AAATACCATGAAGAATCTTAGGAATGCCAGTAACCAGGGAATGGGATATTTGCATATCACAACATCTACA
GGCTAAATACACTCCGAATGCTTCTCAATGTGGGGAAGTATGAGAAGTTAATAATTCTCCTAGTGTCTGG
CTTCCTTTTTCTTTGCCTTTCCCTCTCATACGTTCAATCACCTGTGTTACAGGACACACATAAGTAGTTG
GAAGGTGTTAAACTTTGACAGCAAAGCAAAATCAGAGGTAGAGGCAAATGTTTAACCAATCATGTTCAAA
TATAAGCTACTCAATGAAGAGTTTTAGGTTTAAAGAGCGAAATATTCACCATTACTGAGAAATCTCAGTT
TCTCATTCAATTTCGCATTGCACTTTGCTACCACAAACTAAATGGGTGTACATTTTTGAGTGCTGATTTA
TTACTCAAAGGTTTGAATTTGGAGACTGAAACAAACCATGCTTTATCTGTGTACTTTGAGAAAATCCATC
CACAAGAAAGGAGCCAAGCGTCAGTTTCCAATGGGGGGAACATTCTCATGGAGGTACTGTAAGGCTAAGT
CTGTCCACTTCATTTCTTGTTCTTTGACAGATTCTGTGGTGCCACCATTGTCTTGTTCAGGTTCAGGAAG
CTCATTCTGAAAAGGTACAATAGAAATTCAGAACAGTAATATATTCTGCATCTTCTTGGAACTCAAGAAC
ACTTAGAATTAAAGCTTGTGAGTTCCTAGGAACAGGCAACATAATTGATATGTGTATTCTAAAAAAGCTA
AAATCATACACGTGAGGAAACTATCAGAAGTATCATTCAAACATGGCCCGGGCCTTCTTCAGTAGGGAAT
GATGGGCATTTTGACTCTGACATTTGTGAAGGAGGCACTGTGGCAGAAAGGTTAAGGGCAGAGGCTTTTG
GAAGCCAACTAGGATCCAAGACCACAACTCTCCCATTTGCTGGGTTACTTGGGCAAGGTATGCAAACCCT
TGGGGCCTTTGTTTCTTCCTTGGCAAATGGGAGAACAAAAGAGCCATCACCTCACAGGACTACTGTAAGG
ATTAGGTGAGGTAATTCAGATCAAGTGCTGAGCACTCTGTGGGGTATCCAGTGCTCAAGGAATATTTGCT
ATTATTATTAGCGGCTAGGAACACTGAGATGAGTGAATCCTAAATCTTCTTTAGCGGGAGCGTGCTTTAA
AAGAATACTAGTTTGCTGGCATATTCCTTAGACACAAGAAATGTATCCAAAAAATTATCTTGGACCCAAC
ATGTCTTTTTTTTTTGAGATGGAGTTTTGCTTTTGTTGCCCAGGTTAGAGAGCAATGGCGTGATCTCGGC
TCACCACAACCTCTGCCTCCCGGGTTCAAGCGATTCTCCCGCCTCAGCCTCCCGAGTAGCTGGGATTACA
GGCATGCACCACCATGCCCGGCTAATTTTGTATTTTTTTATTTTTTTTTAGTAGAGATGGGGTTTCTCTA
TGTTGGTCAGGCTGGTCTCGAACTCCCAACCTCAGGTGATCCACCCGCCTCAGCCTCCCAAAGTGCTGGG
ATTACAGGCGCGAGCCACCGCGCCTGGCCCAACATGTCTATATTATAATAAAAATGGAGGTAGGGCAGGA
AACACTGAAAATGTAATTAATAGATAAGACCTCCCTCTAGTGGTGGAGCAGAGCAACAAAAAACACAACA
AATGACCAATTCACACCCTACCAGCAATTTTCTTAAAATGGTAAGTCATGGGCCACACTTACTGTAGGAT
CTGGGATCATTTACTTGTATTTTAAGTTTAAATGTATTACTACTGAAACTCTGCTTTGTACCAGAGTTCA
CAGATACCAATATATTTCTTTTCAATTATCTGTGTTTTGTTTTATGGTTAGCAAACTGTTATGCGATATC
CAGACTTAAAATTTTCCACTGGATTCTAATTTGTAGTATGTGAATATGTTTTCCCCTCTAAAATTATGCT
CAATATATATTAAATGATATTTACTTATCTATATTTGAATCACATACATCTTGTTCTACAAGACAGATTC
AAACATACTTGAACAGATTGAGAAGAGCTATCATTTATTGTAACGCAATAATATATTATCTTAAACTTAA
AAAGAAAGTTCAGTCCTAATAATTACTTATAGATGACAAAGAATAAATAGGTCCTTGGTTAATGAATTCT
TTAATTCAGAAGCAGACAACGTTGCTTAATTTCATCCTATCTGTGGTATGCTTTCTAAAATAATGTGTCT
TTCATTTGGTATCTACTGTCCCTTGCAATACATGCCAACACATAAAATGGCATATATGCCTGGAAAACAA
AAACAAAAGTATCATAAACCCAGCCACCCATTATGGTGTACTGTAAAATGCCCTTAGTTCTTTGCAGCTA
CTCTTATCAAAAGGTGAAACTGATTTCCCCAATCTTGGATTTGGACTGGCCCTGTGAGTTGTTTTGGCCA
ACAGAATGAAGCTGTCATGCCAGTGGGCCAGTTCTTGGCCTAAGCTCCAAGAAGCTCTGCCGTTTCCTCT
CAGGTTGCTCTTGGAACCCTGAGATTGAGAAGTCTGGGCTAGCCTGCTGGATACCAGAGACCATGGCATA
GTCACCCCATCACCCAGCAGACAGCTTGTTAACTGCCAGACATGTGAGACAGTCCTAGATTACCCAGCCT
CCAGAGTCTGTTGGCTGACTGCACAACACAGGCAAGCCTGGCAGGGATCAGCTGAGCTGGTGCAGACTAG
AAGTCCCTCCCAGCTGAGCTACAAAATCATGAAATAAACAGTTGCTCTTTTAAGTCACTAAGCTTTGGGG
TGACTTGTTATGCAGCAAAACCTAACTGGCATACACATTGCCTTAGAATCGCAAGACAAGCGAAAGGTGT
GGATTAGCAGCAGGGCCAAGCCAACTTCGCCTGGAAGTCAGCAGGGAAACCTCCTTCCACCCCTGCCACC
CAAATGTTCTCCAATCTGAGCCATAAACTTCTCCAGAAATGGACGTCTCCTTTATTTTCTTGACTTTGTT
TAGGCCCTCCTCCCTTGCCTAGACCACAACAGCTTGCCAATTGGTCTCACTTGCCCCTATCTCCATGTCA
CACCATCTCTAACAATGCTGGCTAAACTGATTTTCCAAAAGCCAAATCTGAGCATAGCAGTCTTGCTTTT
AAGACCATTCATTCCTGGATCTCCTGAGGATGAGGTCTAAGTGTGATATCAAATACCACTCTCAATCTGA
TCCAAGCCCAACTTTTCAGCCTACTTATGTGACAAATGTATTCTTCATCCACAGTGAATGACTCACCATG
TCCCAAAGCCTTTCAAAACTCTGGGCCTTTACACATCTGTCCCCTCTGCTGCAATACCCTTGTCTACCTC
CACTAAACTTCTGGTCCTTCCTTATGACCCAGCTTAAACATGACATTCTCTCAGAAGCCTTTCTGATGCC
CACCAGGCAATCAGCCACTTCGTATATGATGTGGCTGTTTATGCCCCCACCTAGAATATTTATTATCACA
ATTACTTATCTATAAGTGTTGCCTTTCTAGACCCCTCAGGAGGAGGAACTGTATCTTATGTTCACATAGT
CTTATGGCACACAGTAGGCTTCAAAAAATGCTTGTCAAATGAATGAATGAACAGAAGTTCACAAGACAGG
ATTTGGAACTTCACAGACCTAGGTATATGTTATATGAAGCCCTACTTACCTGCTACCAGACTTTGGGCAA
GTTGCCCTTTCTGATCCTATTCTTTTCCTATAAAATGGGAATAACAGCACCTCCCTCACAGGGTTGTTTA
AGAATTAGAGATAATGTATATTTCCAACCTCAGTCCATGGCATAAAACTGGTATTCAATAAATGACATCT
ATAATTCTTATTATGCTCTCTCTTTTGACTATTTTCCTAAGCTTGAACTAAGCTATGACTTATTTGATAT
GTATATTATTCATTGAACATATTACTATCTCTTATTCGTAAATGAAAATACAGATGAACTATTTTATTTC
TTTGCAAACAGCAACAGCAGATATTCTAACTATAAATTATATTTTATTCAATACTAACTACTCTTCTCAG
GGCAGCTGGCATGCTTTAAAGAAAAACAGTCCTGTTTTCCCTTTTTTAGGCCTCTATGACATAAATAGGA
TTTTAAACAAACATTTATATAAGCAAAACTTTTGGATCACTTATTACATGAGGTTTAACAAACTCCCTGC
AGCAGAGTTTTCCAAAGATGGGGAAGAAAAGAGTATTCCAGAAGGCCACTGGCCTAAACGCAAAAGAATA
CCCAGGTATCTTCTGAATATTTTCCAGGGAACCTAAGATAGGTGTATGCTCCAAGAATGTAAGTTTCTAA
AAATGCTGAAAGACTGATGAGCCACAGGCAGGGAAGTAAGAAACAGAGCTCATATGCTGCTGCTTCTTGT
TCTTCCCCATCTTGCGCATTAAACTCTCATTATTTGGTTTCTCTGGTTTTCGTGTGGTGGACAGAACATG
GCTCAGAAAATCGGGGCTCTAGTCCTGGCATGACTCCTAACTGACCTGGCCATGTTAGCTCCCTGAACCA
CATTTTTCTTCAAATAAAACATACAACTGGATGCTCTCCAGAAGCTTCTAGAGTTAGAGAATTCCACAGC
TTTGATTTCTTTGCCACACTAGTTTATCTTCCATTGATATCCCTCCTCCACTAGTTACTGCCAGATCAAT
ACCTTTAACCCATCCTCAGCTCAAATCATAACCTACCAGTGGTATTGCGACATCCTCATAAGGCAGCTTG
TCTTCTCGCCAAGCATCATCAATCAAATCCAAGATCCTTCCATCTCTCTGAACCTCACTGTCCATTTTCC
TGCAGCTTTGATCTTGTCAAATCTGTAAGCCAAAGAGGTTATTTCTTAAACACGAAGACTGAAGAGATGG
TATAATCCACTATTAGGATTCTCCAGGCTTTTTAGTCTGGGGCCACCTTTTGGGAAAAGGTTCATTTTCT
CATATGGGCCGGGTGACTACTTTGATCACAGTTCCTCTCTCCAACCCCAACTGATACATATTTTATAGTG
TATACTGGACAGGCATAGCCCAGCTGACAGACCAGTGTCATAGAGTGTGACAGGAGAGCAACACGTTAAA
CGAGGCCCTCAAATATTAATATGTTGCATCTCAATGTAGCTCTTCTCTTCTAACTTGAACATTCACCTTT
TCCTGTTTGTGATGCCTTTGATGAGTCCCTGATTCATAATGGCCAGCTAAGTTTCCTTTCTAGCCCCTGG
CTCAAACCTCAGCATTCTTAGGCACAGGAGTCACACGAACATACTGTAAAGGAAACGGCTTCCTTCCCTA
AAATAGTACCCAGGCTGAGATCTGAATAAAGCACTTCTAGGTGAACACCAGTGGTAGAACCTCATACTGC
TAAAGTTTCTCCAGTTTCTGCTTCCCTTTGGGGTAGCTCCTTCCTCGGAGGTTCCTTCCTCTAATTCTTA
ACGCTACACTCCCTTCAATTTCTGGCCCAAATGGTCACGATGCTTATTTAGCTCCTTGACCATGGCTGAT
TCCCCATTTTTCTCTGAAACTGCTATAATCTAATCCAAAAGGCTGGCTAGCTTGTAGGATACTTCTGGAT
TTCCCTGTTTCCAAGGTTGGGCCCTGACTCATCATAGGCATCATCCTATGGGTGCTCCCTGCTACCAAGT
GACAATCCAAAAAGCCCGGAAGCCACTGGCATCAAAGGTACTCTCAGTTTCTGAGACTATGGAGACTCAC
ACAATAAGCAATTTTGGGGTGGTGGAGCGGGGTGGAACCCACCATCTGGAGGTGCTTTTTTCAGAGCTCA
GCCTGGGTGATATTTTAAGGAAGGAAGCCATTTCCTTTACAAGCTCAACTTCAGACATCGTCTTGCCTTG
AGGATTTGTGTCAGGTCTGTAGTTGGTCCCTTCGCATAGTCCAGTTTAGTGTTATCTCAGTATGTTCATG
AGCTCCTGTGCCTAAGAACGCTGAGGTTTGGGCCAAGGCAGGTGGAAGTTTTTGTAAAAATGGCTCACAG
GGCCGGGCGCGGTGGCTCGCGCCTGTAATCCAGCACTTTGGGAGACCGAGGTGGGCGGATCACGAGGTCA
GGAGATCAACACCATCCTGGCTAACAGGGTGAAACCTCGTCTCTACTAAAAATACAAAAAATTAGCCGAG
CATGGTGGCAGGCGCCTGTAGTCCCAGCTACTCGGGAGGCTGAGGCAGGAGAATGGCGTGAACCTGGGAG
GCGGAGCTTGCAGTGAGCCGAGATAGCGCCACGCGCCACTGCACTCCAGCCTGGGCGACAGAGCAAGATT
CCGTCTCGGAAAAAAAAAAACAAAAAAAACCAAAAAGGCTCACAGCTGTCCCCCATAGGATAACTGCAAG
ATATCCCTAACACCTTGGCAAAAATTATACACAGATTGGTGTGGGAAAGGGTTTACACCCCCATGACAAG
GATCTCGGCAAAAGTTAAGCAAAATGCGCCCTGCTACTTCTGTCACCGGTGAGCCAGGTGAGGGCTTAAT
TAATCCAGTACTCAATAATTAACCGAGTACTACCCAGTGGATAACTCAAAGCAAGACGAAGTTTTGGTTA
TACTTTTGAAGTCCCTGTCTTACAAGTGCTCCCCACTTTCCTGCTGCTACTCCTTTGTCCTTGCTGCTCT
GTCTCCTGGGATACCCTTCTCACTTTCCTCAGCTGATCCAAACCTGACTTTCGGAGGCCCACGTAGATTT
TCCACCTTTGTAAAGTTTTCTCAAATCACTCGAGCTTCAGTAGGCCTTCTATCCTTGAACTCTTGCTTCT
TGTCCTAACTTAATCTACTAAACAAGCTAGGTCAGCTTTGTGGTCAATTGCTCAGGTATTATTGCCTCCA
ACTGTTACGTGTACATTTTGTACCCATGATTCCTTAGTAGATCTTTCCACATTTGGTCTCAATCTTTTTC
ACCTGTCCCTTCTCCAAACACCTCCAACCCTGTCACATTTTCCAGCCTTGACAGTTAAAACCCCCTTTTC
CACATCACTAATGCCACTCTTCCTTTCAAGATAAACGCAGGCGTCAGCTCTTGAAGGTCAACATCCCATT
TCCGGGATTAGGCGCGAAGGTGCCTTCCCTCATAAGGGGCTGGAAGGATGGGAAGCATCAAGATAATCAA
TGCCCAGGGCACACTGATTATCTTGTGTATTAAAGCCACCGTCCCAAGTACTCGTCACCCAGTACGACCA
CTGTTTCCTGAACAAACTTTTCTTACTCACGTGACAGTCCCGATGCCTGCAATGCTCTTTGTTTCTTCCT
TTTTATTCCAGTGAACCCACAGTCAGTCTAAAAATCTTAGGTCCCAGGTCGCTTCCTATAGGAGGCCGCC
TCCAAACCCCAGGCTGAGTGCGGTCCCGGTACCCACCCCTCCAGGGGTCTCACAGTTCCCTAAGCACAGA
GCATGGCACCAACTGGGGAGTCTGTCAAACAGCCCAGATGAATGTCTTTCCAAACACGCGGTGCCTGCGT
TTCCAGGGACAGGGACACGTCTTATCAATTTCTATATATTACAGCTTTGTAAACATAATATTTATAAAAT
CAAAGCGGGAAAGCCTTGTTCCTTGCTAGAAATGAGCCTCGCAGGCCACCACCATCCGCACCGTACGACA
GGCCGTCCCTCAGCTGCGGCTTCCTGCCTCAGGCAGGATGTACCAAACGTGGAAGCGGAGACGGGGAGGT
CTGGGGGTGCCACGCAAGTCCCTTTCCTCCCGACACGAGTGCAGTAAAGAAAAACGGTTATGAGCGTAAA
GTGACAGCCTCGGCCGATGGGAATAGGGGGAAGTCCGACACTGAGCAACGAACGCATTCCCGCGCCTCCA
AAACCTAGGCCGGGGGCGCTGGAAACCCTTACCGGCACCCGGCCACCGCGGCAGACGCTTGCTCCTGCCA
CGCCCCCCCCCCCTCCCCCGCATCACGTGTCTGCACTCGCTTTCCTCGGATTCCCGGATGTGGGTGAGTT
CATTTGCTCGCGTGCAGGGGAAGTCTGGAGAAGGCATTGTTTCAATTATTAAAAGTGTGGGGGCAGTGGG
CGGAACAAACGCGCCGACTACAGAGGCTGGACGTAAGCTTAGCGGTGGCGCGCGTGCGCAGCGCCGGCCC
GAGGTAACGGCGGGAAGGCTCAGGGGCGTGCTTGCGGCAACCCTGTAACCGCCGGTGCGCAAGTTGGAGG
GGCAAGGGGCTGCCGTGTCCTGGTCTGGAGGCGGCCCAGGCTGCCTTGGTGATTCTGGCTTCGCGGCCTC
ATGGCTTGCGGCCGGTTTGCGCAACGGCCTGCGAACCACCAGGCGCGTCCCCGCCGGCTTGGGTCCGCCC
CGAAGCCCAGTCCCTCGGCCTGGCCCGCTATGCCCTGCACACTGGCGGAGTCTGGCGTGGCGCAGCCCGG
CGTGGGGTTCGGCCCCGCCAGGTGGTCAGCCCTTCCTCGGAGTCCAGGTGGCTGGGGGCGCTTCGGAGAG
TGGCCAGGTGGGCGAGCGAGCTGCGCCCAGTACTCACCTTCCCCGGCGGACCCACCTTCCTTTTCAGCGG
AGAGACCCCAGTGCGGCCTCCCTGCCAGGGCTGCTGTCTAGCTAGCGCTGTTGGGACTACTTCATTCACG
GCACAAGGGGACCCGCTCATTTAGTGTGGCATGTTAAGGTTGGCAGCTTTTAAAATAGTGAGTTGGTTTA
AACGGTTATGTTAGTACAAGAGGTACGGAAATGTGGCATAAATCGTGGACTGAAATTGGAGAACCCTGAA
CTAACGGCTCCATGCACACAAATGAGGGATAGAAAGAAATAAGGACGGTTTCTTAGGAACACTTTAATCA
TGGAGATACTTGAAGACAGTACATTTAGGCTTATAATGTGGTTTTTCTTACGTCCCTTACGACATCAGGA
GAGTAAAGATGAGTATGATGTCAGAGACAGGTCGTCTTTTACAGTTTCAGCACCGTACGCTGATATGCAC
AGAGTAGTTTTCCCTGAGCCTTATGTCTGGTTAAACTTATTAAAAGAATTTATTATTTTAATAAAGCAGT
ATTGTAGGGTAGATGCCCTGTATTTATACAGTTCCTCAAGTGCTTGCATTTATTTTTTAGATACGAGGAA
ATCATAAGGATCGTTGATTAGATTTCTGTTAGCTGTAGCTTAATGATAAAGTGTATATATTTTCTTATTT
GGCATGCAGTACCATTTTTAGATTGATTTTTATTTTGAGTTTGCTTTTTGACTGTGGCATTATCAGTGAT
CAGGGTAGTAAAAAGCTCTGAAACTAAATTAGAGTAGAGATTTATAAACAGGCACACTGCCTTTTAAAAT
GTATGTAGATGCAAATCTAAAAGTTGTTTGTAAAAATTGTGTATGTTGATTGTACTGATGCCATTTCCTC
TCGTTAAAATGGTGGAAGACCTTTGCTTAGTCCTTTCTGTTCTAATTTATGTTGTGCTGGCAGATGGTGA
CAGCATCAGTAGTCCACTCTGTCATTGCTTCCTGCTTTCAAAAAATGTCAACTTGACTTGAAATTCAGCT
AAAGCCAACATTGACAGAAGGTTTCAGGAACTGAATCTCCGATCTTTATGTTATTTTAGTCCGCCTCCCC
GCCCCCATCAGGAGCTAGGTTTTTCAGTTTTACAGAGCCACAGTACTGTCATTACACATACACAAATTAA
TAAAAATTCTCTAATGTCAATATCCAGTCAGATAACGATTTCTTCAACTGTCTCTTAAATGTCTTTTTAC
AATTAGCTTGTTTGAACCAAAATCCCACAGGGATCTGCCTATTGTGTTTGGGTGATGCACATTTGACATC
TTTTAATCTATATTTGTTCCCTCATAGTTATAGCTATGGGATTAGCCCCTATTTTTCCTTACAGTTCTAA
ATTTAGCAGATTGCATCGCACAGTATCCTTTAACATGTATCTCTGTTGCCTTTGTGTCCTCTAAACCAGG
GGCCCCCAAACCCGGGGCCGCAGAGCAGTACCAGTCCGTGGCCTGTTAGGAACCAGGCTACAGAGCAGGA
GGTGAGTGACAGGCTAGTGAGCATTACCTACCACCTGAGCTCTGCCTCCTGTCAGATCAGCCGTGAGGTT
AGATTCTCCTAGGGGCACGAACCCTATTGTAAACTGCGCATGCAAGGAATCTAGGTTGCACACTTATAAG
AATCTGACTAATGCCTGATGATCTGAGGTGGAATAGTTTCATCCTGAAACCATGCCCCTAACCCTGTCCA
TGTGTATTATTCATATTACTGAAGAAGTGGTTGGCCGGGCGCGGTGACTCACGCCTGTAATCCCAGCACT
TTGGGAGGCCGAGGTGGGCATGAGGTCAGGAGTTTGAGACCAGTGTGGCCAACATAGTGAAACCCCGTCT
CTACTGAAAATGCAATAAAATTAGCCGAGTGTGGTGGTGTGCGCCTGTAATCCCAGCTACTCGGGAGGCT
GAGGCAGGAGAATCACGTGAATCCGGGAGGCGGAGGTTGCAGTGAGCCGAGATCACACCATTGCTCTCCA
GACCAGGCAAGAGTGCAAGACTCGGTCTCAAAAAACAAAAAAAAGTGGTTTGGCCATTCCTCCCCCAAAA
CATTTCATATGAATTTTAACAAAAAGATATTTACAAAATGTGTTATTTCCGCCCTGTCGGTGTTGAGAAA
TTGTCTTCCATGAAACAGGTCCCTGGTGCCAAAAAGGTTGGGGACCACTGCTCTAAACCAGTAGTTAGAT
GTAGAATTGCACTGTCCGGTATTGTAGCCACCAGCCAGCAGTGGCTAGTTAATTAAAGTGAAATAAAATT
AAAAATTCAGTTCCTAGGCTGGGCGCGGTGGCTCACGCCTGTAATCCCAGCACTTTGGGAGGCTGAGGTG
GGTGGATCACGAGGTCAGGAGTTCGAGACCAGCCTGACCAACATGGTGAAACCCCGTCTCTACTAAAAAT
GAAAAATTAGCCGGGCGTGGTGGCATGCGCCTGTAATCTCAGCTACTAAGGAAGCTGAGGCAGGAGAAGG
AGAATCGCTTGAACCCGGGAGGTGGAGGTTGCAGTGAGCCGAGATCACGCCACTGCACTCCAGCCTGGGT
GACGGAGCGAGACTGTCTCAAAAAAAAAAAAAAAAAAAAAATTCAGTTCCTAACTTGCATTAGCCACATT
TCAAGTGCTCGGTAGCTACAGATTGCCTGGTGGCTGCTTTTTGAACAGTGCAGTTATAAAATATTTTCAT
AATTGCAGAAAGTTGTATTGAACAGCACTGCTCTAGAAGCCTGATGAGATTTCACCCTTATATTTGTTTT
TGTTTTGTTTTTTGGTGGGATAGTTGGGGGGGTTGGACAGATAATTTCATAGGTAATGCAGTATACTTCC
TATTTCATTAATTATCAGGGAAGCAGTGTCTGAGTTGTTTTCTTTTTTTATGATGTTAACATTAAACATT
ATATTGGGTTTAGGTGTAGTTAGCTCCATCCACCCATAGTGAAATTCCCCAAGACTCTTTCACTTAGTGG
TGAAGTCCTTTTGTGGGGTTATATTGCCTGTATCCGTTATTGCATTAGAGGTTGCAACGTGGTGATTATC
TAATTCTATTATTTCTTCAATATTTATCAACTGAGAGTCTTTTATGAAGAACTTTCCCTTTCCAGCTGTT
TGACTACCCTGAAATACAATTTGTACAGGAAAGGCAAGAAAAATGCTTAATTCTTTGCCTCTATTTTCAG
ACCACAGTAATGAGTTGGTGGCCTAGCAGCCTCCAAAGGTAACCATGAGGGTTTTTGCCTTTTTGTAAAC
CTATAATCATGAACTTATAGACTTTATATTTAAACTGTTTCAACATATGGCAGTTGTGTTTTTAATACTC
AAATTGTTTCATCTTTGGCCAGTGGGAGCTCCTTAAAGTTGGCTCCTATTCCATTTGATATGACCAATAG
CTTTGGTAATTTCCTTGCTTTCTGGTAAATAATTTTCCTGGCTCATTTATAGATTTCTGGTCTTCAAGGA
GCCGTGGTTCTTTTGAGTCTGAGATAGTATTTGGAGACCACAGTCTTACCTAGGTGGGAGAGGTGTTCTT
GCTACTAGGTTAGTCGTTAGTTCTAGGATTTATGGTGGCCAGAGTTAGGAAATATTATATATTAAAAAAA
ACACAAATATGCAAAATTCGCATTGTTTCCAACATTTAACATTTAAATTTCCAAAATTTAAACTTAATTT
TACAGTTTTTTCACACGTTGGATTTATGTTTGTATCTTTTTTGCTGAGTCTTGGTGCCTAGTGCCATTAA
TAGAATTAGTTTTTTTTCCTCATCCTATGTTATATATAATGGTTTCAAGTTAACAGGCTATTTTATTATC
AATATGATTCCTGAATAGTGCTTAGGATTTCTTTGCAACTGTCTTTTATCCTTATTTTATATGTCTCTAG
GAAAGTGTATAGTAAAATTATTGTATTTAAAGGCATATGAAGTAATTTATTGTATAGTTAGCCTATCAAG
TTGATATCTAGTTGGTAGTTGATTTATTTTATTTTGTTTCCAATTTTTCAGTACTGCTTTTTTCCCCATT
TTGATTTGTTTTATTATATGCAAAACCTTAGCATAATTCCCATAAAACAAGTACCCATAAAACAATTTAG
CTTTCATCTCTGTTTCTTCCACTCTGCTTATTTTCCTTCCTTACTGATACTGTTTTTACTAGTTTTGAGT
CTATCCTTCCATTAAAAAAACACATACCTAAAAGCAAATCTACATGGACAAACATACCTTTCTTTCTTAG
GTAAAATGTGCTATTATGTATACTGTTTTCCATCTTGGGTTTTTCACCTAGTGATATATGTCCTGTAGAT
CCCCTCCTTAGGAGTATGTAGAGATTGTTATTCCTTTTTACAGTTTCGTAGTACTCCATTGGTGGATGAA
TATTTAATTCGTCCCCTCTTGATAGACACTTGTGTTATTTTCTGCTATTACAAATAGTCATGCAGTGAAT
ATCCCTGTGGATATGTTGTTTTGCATTTTTGCCAATGAGCCTGTGGGATAGATTCTTAAAAGTAGCGTTG
TTGGATAAAAGGGTAAATGCATATATCAGTTGTTAGGTATTACCAAAGTCTCCTCTATAGCAGTTGTGTT
CATTTTGCATTCCTACCTATTATGTATATGAGTGCCTGTTGTCCCACAGCCTTGTCATCAGAGTGTATTA
TCAAACATTTAATTTTTGGTTAATCTGGTACAGAAGAAAGGGTAACTTACTGTAGTTTTAATTTGTATTT
CTCTTATGAGCAAGGTTGAACATCTTTTCATATTTTTACTGTCATTTGTATTTTTCTGTGCATTGCTTGT
TTATACCTCTCGCCCATGTTTCTATAGATTTTTTGCCTAATCTTTTATAAGCTCCTTATATATTTGGGTA
ATTAACCGTTTGTGGTAAAACTTTCAGGTACTGTTGTCATTTGTCAGTTAACTTTGCTTATGGCACTTTT
TCATATAAAAGTTTGTTTTTATGTACTCCTATTTCTCAGACTTTCTCATTGTTCTTCCTGGATTTTGAAC
TATAGTTTGGACAATTTTTTAAAAACTTTAATATTTTGAAAAGTTTCTCTAATACCTTATTTACTGGTAT
CAGTTTAATTAGGAGCCTTTCTGGAAAATGCTTAGTAATACTGATTGTTCATCGGAACAGCCATAGTTTA
CTGGCCATGGTATATCTGACACAATCATAGAGCTTTGTAATGAAAACGACACCAAGGCAGGTACAGGGAC
AGAGAGCATTGTTATCCTCCTTTAGACTTGAAGGTTAGGCATATCCCTTCTCTTAGTATGTACACATTTA
TTCATAATTCTGTCTGGAGTTTTTATTTTCTTCCTGTGTCCCATTTTGTGAATAATTTGGTCTTGGGCAT
ATACTATGATATTTTTCCTGATCTGTAAAATGATAAAGTTAAATTAGAGGCCTGAAGTGCTTTCTAGCTC
ATAAAGTCTATGTTAATGAATTTCAAAGATTTGCCTATTATAAGACATTCTATTTGTATTCTTTTTTTTT
TTTTTTTTTTTTGAGACGGAGTTTTGCTCTGTCGCCCAGGCTGGAGTGCAGTGGCGCGATCTTGACTCAC
TGCAAGCTCCGCCTCCCGGGTTCACGCCATTCTCCTGCCTCAGCCTCCCGTGTAGCTGGGACTACAGGCG
CGCGCCACCATGCCCGGCTAATTTTTGTATTTTTAGTAGAGACGGGGTTTCACCGTGTTAGCCAGGATGG
TCTAATTTTATGTAATTATATGGCACATGCAAAGAAGAAATCAAAGTGACATAGTGCATAGAGTTGAATG
ATGGTCAGGTTGGTTAGGAAAAGAATCAGTGATATAACTATGAGCAACTCAGATGACAAACATCCACAGA
AAATCAGCCCTTACTATTTGCCTGATCCTGAGAATTGATGGTGTTTGGCACCAGGCAGATGGTGAAGGTA
TTACAGAAACACTTTGCACATTAGTAAGTTTACTTTATTTCTCACATAGGATATTACTGGTCCACTTCCC
TCCTTTTTTTTTTTTTTTTTAAACAGGAATCTCTATCTTAGACCATTTTGATTTCTAAAATAGCAATATT
TCTTATGTGACTATTTCATTATTGTGCTGCTGCCACCTGGTGGCCACAGTAGTGATGTCATACTTCAGTT
TAATTTCTTTTTTTATTGCAAAGACACTATTAAGCCAAAAGATAGGCTGATATATTTTAAGATATAGAAT
ACTGTAAAATATATAAAGGTTGCACATCGATTGTGACTCAATTGATTGCCACAATGTCTAGGTTGAATCC
AGGACTATAATTTTTAGTTTATCATAGTCAAGTTATTTAACTCTCTAAGCCTGTTTTCTCAAAGTAATAG
TATCTGTCTTTTATGAGTATAGTGAAGATTGATTTAGACAATGTGTACAAGGCAGCTGTTGAACATTCAG
TAACTTAACTTCTAAGTATATTATTAAATAGTCATAGAAAATATATTAATGTATATTACTAAATATATTA
ATGTTTAAGAATAACATGACCTAAATAAGTATAATTTTACTAATGATAATTTTGAATAACTGTGATAGCA
CTGTCAATAAGAATAGCACTTTCCTACATGTTAGTTAAGTATCCGTGTATGGTTGTCAGGAGCATGCTAC
TTACAGTGGACATTTAATTGGCTGTTAGAACCCATGAAAAAGCTGGAAGAAACCTTAGAGAATATTGTTC
ATGCCCCTCATTTTGCAGATGTTAAAACAAGTCCGAGAATATAAAGCTAGTTAGTTGCAGAGTAGGACTG
TATCCTGGGTCTCCATCTAGTGTGGTGTGGACCGTGTACTGCCTCCCAGTTCTCCTAGAGTGGACCAAAA
CATGGAATCTTGGTTGCATTGTAGTTGGACTGTATTGTGATTTGAAAGTGGAGTAAAACATGGGAACCTT
TTTCTTTAGGTTTTTATTTTTGCAATTTTGGTGTAATATGTAGGATAAACCAACTAGAAAAAAAATCCTC
CCTGTCACGTTTGTGCACAAATAATTTAATACTCAGTTTGTTAGTATCTTTAAAAATAGATGCAAATAAG
GTGCAGTGGTTCTATTTTCCAGTCTTCTCATTCTCCTCTGTGTCTCTGGAACACCCTGGGCATGTCTCTC
ACTATATTTATCACACTGGGAAAGCCTTTTTGGCTCATTTTTTTCTGTCCCATACTAGACTATTAATACC
TTGAAAGGGGCCATATCTTTCATCACTATATATCTAGTTGTTAGTACAACATTGAATTACGTGTTGAGTA
AAATAATCTACTTCTTACAGCAGTATAATTTTTAGATGAGGATTAGCCCAGGGGCAGAACCTCTACACCA
CAGTCTGGTTATTGTCTTAAGGGTTTAAGGAAAAAGCATTTTGGAATGAAAAGTGGTTTGACATTGAACA
TTTTGAAGATTTATTATAAAAGGCGAGAAGCATACACATATTAATCAACACTACTGAGTTTAAGGGTCCT
CTTTCCTTTCTCACTGTCACCTTTCTTCATGGTGTATCTTACCCTTTATTTTATATTTATTTATTTATTT
ATTTATTTATTTATTTATTTATTTGAGATGGAATCTCTGTCTGCCACCCAGGCTGTAGTGCAATGGTGTG
ATCTCGGATCACTGCAACCTCCGCCTCCCGACTTCAAGCGATTTTCCTACCTCAGCCTCCCCATGGCTGG
AACTACAGGTGCATGCCACCATGCCCAGCTAATTTTTTTTTTTTTTGGTAGAGGCGGAGTTTTACCATGT
TGGCCAGGCTGGTCTCAAACTCCTGACCTCAGGTGATCCGCCTCCCTCGGCCTCCCAAGTTCTAGGATTA
CAGGCGTGAGCCACCGCACCCAACTGTATCTTGCTGTTTATATGGCAGTCTTCTATCTCCCGTTCCCTCT
TTCTCTGCTAGACCCTTCATGTAGTTCCCCAGCTGTGACCAGTTTGCATGGGGGAGAGGGTAGGTAAAAG
AGGGAGGGGAGTTAAGTTCAGCCAAAATAATCTGCCACGTGCCATGTTGAAATGCTGCGTTGAGCTCTGC
AGCTGCAGAGATGAATAAAATTGGGTTTCTGCCCTGGAGGGATGAATAGTCTAGTAAGGAAAAATAGTAT
GTAAAAAACTCACTTCCAGTGTGGTGCAACTCCTATGAAAGAACGTATTAAGCACTGGGAGCAAATGAAG
TGCAGAGGTGGGAGGGCCCTGTGGCTGCCTTTCGGTGGAGAGAGGATGTTGCATGCAGGAAGCTCTATGA
GCCTGTCTTCAGGAGTGAGTAGGCTTTCCCATGATAGACCAAAGAGTTTAATCTCAGTACACTTCGCATT
TTTTATACTATCTCTTTAATTTTTAAAAAGCTATACTTTCCCTCTGACATGTGTGAACCCATTTCTTTCT
CATTTTGTTAAGGGAGTTTGTGTATCCGTCTGCTAGAGAGATCCCACCTCCACCCCTGCTCACCCGACAT
TAGGAATTAGGCAAAGAACAATGAGATATTTGATATTTACACGGAGAGAGTGTCAACCGCAATTCAGATA
TTTTTAAGTTATTCTGTTTTAGTCCGAAATAGTTTGTTCCTAGTATTCCTGAAGTGATGTTCTTTGACTG
CTTCTACAGTCATCTTAAGAAGTGCTGCATTTTTTCATTATTATGTATGCTTTCATTCACATTCTTAAGT
TGTAAGTTAGAATGTTAGAACTGAAGAAATGAATTGTCTCATGACTGATATTTTTTTCTTTGTCAGTTGC
CAAAACAAAGGGGATTTGGTGATGGAGGCTTTGTTAGAAGGAATACAAAATCGAGGGCATGGTGGGTAAG
TTTGCTTTTTTTAAAATTAATTTTTTTGGTTAATACATGATTACATATTTATAGGGTTCACATGATACTT
TGATACATACATATAATGTGTAATGATCAAATAAGGGTATTTAGTATACCTATCAGCTCAAGCATTTATC
ATTTTTTGTGTTGGGAACATTTCAATTCTTCTAGCTACTTTGAAATATATAGTAAATTTTTAAAGTTTAA
GGAACCTTACTGTGCTAGTCACCCGACTGTGCTAATTACTTAACTAGTTACATACTAGTTACTTAGTAGT
CACCTTACTGTGCTATTTAACACTAGAGCTTACTCTTCTACCTAACTGTATGGTTTGTACCCATTAACCA
AACATTCTTCACCCCCAACCCCTTCCCAGCCTCTGGTAACCATCGTTCTACTCTCTACCTCCATGAGGTG
AACTTTTTTAGACCCCACATATGAATGAGAACATGAGATATTTGTCTTTCTGTGCCTATTATTTCACTTA
ACGACCACCAGTTTTATCCATGTTGCTGCAAATAACAGGGCTTCATTCTTTTTTATGGCTGAATAGTATT
CCATGATGGATATATTTCTGACATTTTCTTTATCCATTAATCTGCTGTTGGACACTCAAGTCAATTCCAT
ATCTTGGTTATTGTGAATAATGCTGCAATAAACATGGGGGTACAAGTATCCCTTTCACATACTGATTTCC
TTTCCTTTGGATAGATTACCCAATAGTGTGCTTTCTGAATCATATGTGTTGTGAGTGGTGCAAAGTCAGA
CATAACCAGGTCCATACATGTTTGTGTCTTTTCTACAAGAGCAAACTTTTATTGATGCTATTTCAGTCAT
AAAAGCCACAAGCTACATGGAGTTCCCAAGGAGGCATTTCCCCCTAGTACTACCAGTTCACTCAATAGTC
AGAGCCATGGGCATACAGGCTCAAGCCTCTCCACAAGTCAGTCAGTATTGCAAACCATACATAATAGTGT
ATTAATATATGTAAATGTTGCAGTGTAAACATTCCACATCAAACAAAGTAACATTTAACAGGAGAAAAGG
ATAGAAAAAAAAGGGGGGGGGGGCTAAAGAACTAGTCCAAAGAGAACAACTGGACAAAGAGAATATTCTG
GCCTGATCTGGACGGTTGTCAACATTCTTGCAAGGAGGAGCCTTTGGTGACAGATGCCAGGTGCTGATCA
CAAGTGACAGCCAGACAGGCTGTTATCAAGACAGCTGTTTTGAGCTGGTAAAGTCTTGCTCTTTTTGTGG
CCACAGAATCTTCTGGTGATAACTGATAGTGAAATATTGTGCCTGTTTATGTCATTATCTGATTGAGTGC
AGTCTTTATTGATAAGGTGAACATCGGGTCCCTGTGGGCATGATGTCTTTTGAAATGTGAGATGCAATCT
CTTTCTAAAATGGAGTTACTTATGTTAAGGGTGCTATATACAATATGGTAGTTCTATTTTTAGTTTTTTA
GAAAACTCCATTTAGTTTTCCATAATGGCTGTACTAATTTTACATTCTCACCAACAGCATATGAGAGTTC
CCTTTTGTCCACATCCCCCCCAGCATTTGTTATATTTTGTCTTTTTGATAATCATTCTAACTGGGGTGAG
ATGAATCATTGTGGTTTTCACTTGCATTTTCCTGGTGATTTAGTGGTGTCGACCATTTTTTCATACACCT
TTTGGCCATTTGTATGTCTTCTTTAGAGAAATGTCTATTCAAATCCTTTGCCCACATTTTAATAGGGTTT
TTTGTTTTTTTGCTGTTGAGTTGTTTTTCTTGTATATTCTGGATGTTTAGTCCCTTGTTGAATGAATAGT
TTGCAAATATTTTCTCCCATTCTACAAGTTGTCTTTGACTCTATTGTTTCCTTTGCCGTGCAAAAACTTT
TTAGTTTAACTAGGTCCCAATTGTCGGTTTTTGTTTTGGTTGCCTGTGCTTTTGAAGTCTTAACCATAAA
ATCTTTGCCTTGACCAGTGTACTGAAGCATTTCCTCTATATTTTCTTTTTGTTTTTTTAGAGACGGGTGT
CTCATTCTGTTGTGCAGGCTGGAGTACAGCAGCACAGTCGTAGCTCACTGTAGCCTCAAACTCCTGGGTT
CAAGTGATCCGCCTGCTTCAGTGTCCCAAGTATCTGGGACCATAGGCATGTCATCTGGCTAATTTAAGAA
GGTTTTTTTTTTAAGAGATGAGATCTCATTGTGTTGCTCAAGCAGTTCTTGAACTCCTGGCCTCAAGTGA
TCCTCCTGCCTCAGCCTCCTGAGTAGTTGGGATCACAGGCATCAGCCACTGTACCCAATCCCTATGTTTT
CTTCTAGTAGTTTTATAGTTTTGGGTCTTACATTTAAGTCTTTAGTCCATTTTGAGTTGATTTTTTTTTT
AATATGGTGAGAGATAGGGGTCTAGTTTCATTTTTCTGCATGTGAATATCAATTTTTTCCAGCACCATTT
ATTGAATAGGGCGTCCTTTTCCCATTGTATGTTATTGGCACCTTTGTTGAAAATCAGTGGGCTGTAAATA
CAATTTCTGGGTTGTCTATTCTGTTCCGTTGGTGTATGTTTCTGCTTTTATACTAATACCATGCTATTTT
GGTTATTATAGCTTGGTAGTATATTTTGAAGTCAGGTAGTGTGATACCTCCAGTTTTGTTCCTTTTGATC
AAGATTCCTTTGGCTATTCAGGGCCTTTTATGGTTCGGTGTGAATTTTAGTATTATTTTTTCTATTTCTG
TGAAGCATGTCATAGGTATTTTGATAGAGATTGCATTGAATCTGTGGATAGATTTGGATATAATCATTTG
AATATATTAATTCTTCTGACCCATGGGCATGAATGTCTTTTCATTTGTATCTTCAATTTCCTTCATTAGT
GTTTCATAGTTTTCTTTGTAGAGGTCTTTTATGTCCTTGGTTAAATTTAATCCTAGGTATTTTTTTTTTC
TGGTAGCTATTGTAAATGGGATTGCTTTTTTGATTTCTCTTTCAGCTAGTTCATTATTGGTGTATAGAAA
CATTACTGATTTTTGTATGTTGATTTTGTATTCTGCAACTTTACTGAATTTCTTTATCAGTTCTAAGACT
TTTTTGGTGGAGTCTTTAGGTTTTTCTGTATGTCAGATTATGTTGTACACAAAGAGCAACAATTTGACTT
CCTCCTTTCCAATTTGGATGCCTCTATTTCTTTTTCTTGCCTGATTGCTCTGACTAGGACTTTCAGTTAT
GTGTTGAATGGGAGTGGTGGAAGTGGGCATCCTTGTTGTGTTCAGGTTCTTAGAGGAAGGGCTTTCAGCT
TTTCCCCATCCAGTATAATGATACCTGGGGAGCTGTCTTATATGGCCTTTATTATGTTGAGGTATGTTCC
TTCTATGCTTGAGAGTTTTTATCATGGAGAGATGTTGAATTTTATCAAGTGCTTTTTCTGTATCTGTTGA
GATGATCATATGGTTTTTGTCTTTCATTCTGTTGATGTGATGTATCACTTTTATTGATTTGTATATGTTG
CATACCTGGGATAAATCCCACTTGATCATGCTGTATTATCTTTCTGTTCTGTTGTTGGATTTGGTTGGCT
AGTATTTTGTTGAAGATTTTTGCATCTGTGTTCATCAGGGATATTGGCATGTAGTTGTCTTCTTTTGTTG
TGTCCTTGTCTGCTTTTGTCATCAGAGTAAAGGTGGCCTTGTAGAATGAGTTAGGAAAATTTTTCTCGCC
TTCAATTTTTTTGGAATACTTTGAGAAGAATTGATGTTCTTCTTTATGAAGTTGGTAGAATTCAGCAGTA
ATGCCATCAGGTCCTGGGCTTTTCTCTCTTGGGAGACTTTTTATTCCTGGTTCAGTCATGTTACTCATTA
TTGGTCTTTTCAGGTTTTCTAATTCTTCCGAGTTCAATCTTGGTAGGAATTTATTTCCTCTAGGTGTTCC
TATTTGTTAGCATATAGTTGTTTGTAATAATCTCTAATGACCCTTTGTATTTCTGTGGTTATCAGTTGTA
ATATTGCCTTTTCTGTTTCTGATTTTATTTATTTGGGTCTTCTCTTTTTTCTTGGTTAGTCTAGCTAATG
GTTTATCGATTTTGTCTTTTTAAAAAACCAACTTTTTGTTTCATTGATTCTTTGTATTGTTTTTTAGTCT
CTATTTTGTTTATTTCTGATCTGATCTTTGTTATTTTTTTCCTTCTGCTAATTGGGTTTTGTTTGTTCTT
GCTTTGCTACTCGAAGTGCATCGTTAGGTTGTTTATTTGAAGTCTTTGTACTGTTTTTATTTTTTAGATT
CAGGGGTACATGTGCTGGTTTGTTAAATGGGTGTATTGTGTGATGCTGAAGCTTGGGCTTGTATGTATCC
TATCATCCAAAGAATGAACATAGTACACAATAGATAGTTTTTCAGCCTTTGCCCACCCTCTGCCCTTGCC
TTCTTTTGGAGTCTCCAGTGTCTGTTGTTCCCATCTTCATCTTTTTTTTTTTTTTTTTTTTTGAGATGGA
GTCTCGCTCTGTTGCCCAGGCTGGAGTGCAGTAGCATGATCTTGGCTCACTGCAACCTCTGTCTTCTGGG
TTCAAGTGAGTCTCCTGTCTCAGCCTTGCAAGTAGCTGGGATTACAGGCACACACCACCATGCCCAGCTA
ATTTTTTTGTATTTTTGGTAGAGATGGGATTTCGCCATGTTGGCCAGGCTGGTCTCGAACTCCTGATCTC
AAGTGATCCCCCCGCCTCAGCCTCCCAAAGTGCTGGGATTACAGGTGTGAGCCACCATGCCTGGCTTATT
ATTCCCATCTTGATGTCTGTGTGTACTCAATGTTTAGCTTCCACTTGCAAATGAAAACATGCAGTATTTG
GTTCTCTGCTTCTGTGTTAATTTGCTTAGGATAATAGCCTGCAGCTGCATCCACATTGCTGCAAAGAACA
TTTTGTTTTTCTTTTATGGCTGTGTAGTATTCCATGGTGTATATGTACTACATTTTCTTTATCCAGTCCA
CCATCAATGGGTACATAGGTTGGTTTCATGTCTGCTATTGTGATTGGTGTTGCGATGAACATGTGAGCGC
ATGTGTCTTTTTGGTAGAATTATTTATTTTCCTTCGAGTATATACCTGGTAATGGAATTGCTGAATCGAT
TGGTAGTCTCATTCTATTTTTAGTTATTTGAGAAATCTCCAAACTGCTTTCCACAGGGGTTGAACTAATT
TACATTCCCACCAACGGTGTTGCCTTTTCTCCACCGCCTCACCAACATCTGTTATTTTTTGACTTTTTAA
TAATAGCCATTCTGACTGGTATGAGGTGGTATCTCATTTTGGTTTTGATTTGCATTTCTCTGATGATTAG
TGATGTTGAACATTTTTCAAGTTCCTTGTAGTTTATGGATATTAGCCCTTTGTTGGATGCATGGTTTGCA
AATACGTTCTCTCATTCTGTAAGTTGTCAGTTCGCTCTGTCGGTAGTTTTTTTTTCTGTGCAGAAGCTCT
TTAGTTAGGTCCCAATTGTCAATTTTTGTTTTTATTGCATTTGCTTTTGAGGACATATCTTTTTGAGTCA
TAAATTCTCTGCCTAGGCCAGTGTCCAGAAGAGTATTTCCTAGATTTTTCTCCTAGGATTCTTATAGTTT
GAGATCTTACATTTAAATCTAATCCATCTTGAATTAATTTTTGCATATGGTGAGGAAGTGTAGGGGTTGA
GTTTCATTCTTCATACAAGTAGCCAGTTTTCCCAGCACCATTTATTGAACAGAGTATCCTTTCTCCATTG
TTTGTTTTTGTCAGCTTTGTTGAAGATCAGTTCATTGTAAGCGTGTAGCTTTATTTCTGGTTCTCTGTTT
TGTTCCATTGGTCTATGTGTCCATTTTTGTACCAGTATCATGCTGTTTTGGTTACTATAGCATTGTAGTG
TAAAGCAAGGCAATATGATGCCTCTAGCTTTGTTCTTCTTGCATAGGGTTGCTTTGGCTATTTGGGCTCT
TTTTTGGTTCCATATGAATTTTAAAATAGTTTTTTTCTAATTCTGTGAAGAATGACAGTAATTTGATAGG
AATTGTATTGAATCTATAGGTTGCTTTGGGCAGGACGGCCGTTTTAACTGTATAAATTCTTCTAATCCAT
GAGCATGGAATGTTTTTCTATTTGTTTATGTCATCTGTGATTTCTTTCAGCAGTGTTTTGTAATTCTTTT
TGTAGAGGTTTTTCACCTCTGTAGTTAGATGTATTCTTAGATTTTTTTTTATGTGTGGCTATTGTAAATG
GGATTGTGTTCTTGATTCAGTACTCAGCTTGGACGTTATTGCTGTATAGAAATGCTGCTGATTTTTGTAC
ATTGATTTTGTATTCTGAAACTTTACTGAAGCTTGTTTATCAGGTCTAGGAACATTTTGGTAAATCTTTA
GGGTTTTCTAGGTAAAGAATCATATCATCAGCAAAGATAGATAATTTGACTTTTTCTTTTCCTATTTGAA
TACCTTTTATTTCTTTCTCTTGCCTGGTTGCTCTGGCTAGGACATCAAGTACTAGGTTGAGTAGGAGTGG
CAAGAGAGGACATCCTTGTCATGTCCCAGGTGTCAAGGGGAATGCTTTTAACTTTTGCCTATTCAGTATG
ATATTGGCTGTGGGTTTGTCATAGATGGCTCTTACTATTTTGAGGTCGGTTCCTTCGATGCCTAGTTTGT
TGAGGGCTTTTAACGTGAAGGGATATTAAGTTTTATTGAATGCTTTTTCTGCGTCTATTGAGGTGATCAT
ATGGTTTTTGTTTTTAATTCTGTTTATGTGATGAATCACATTTATTGATTTTTGCCTATGTTGAACCATC
CTTGCATCCCAGGAATAAAGCCCACTTGATTGTGATGGATTATCTTTTTGATGTGCTGCTGGATTCAGTT
TGTATTTTGTTGAGGATTTTTGCATCTATGTTCATTAGGGATATTGGTCTGTAGTTTTTTTTTGTTTCAT
CTTTGCCAGATTTTGGTATCAGGGTGATGTTGGTTTCATAGAATGAGTTAGAGATGGATCCCTGCTCCTC
AATGTTTTGGAATAGTTTCAGTAGTGGTACTACCTCTTATTTATATGTCTGGTAGAATTTGGCTGTGAAT
CCATCTGGTCTGGGGCTTCTTTAGTTGGTAGGCTTTTTATTACTGATTTAATTTTATAACTCGTTATTAA
TCTATCGAGGATTTCTCTTTCTCCCTGTTTCAATTCTGGGAGGTTGTGTGTTTCCAGGAATTTTTCCATT
TCCTCTAGATTTTCTAGCCTGTGTGCATACAGATGTTCATAGTAGTCTGTGAGGATCTTTTGTACTTCTA
TGGGATTGGTTGTAATGTCACCTTTGTCATTTCTGATTGTACTTATTTGGATCTTCCTTTTTTTTGGTGA
TTTAGCTAGCAGTCTATCAATCTTGTTATCAACTGTTTATTTCATTTGTTTTGGTCTCAATTTTGTTTAT
TTCTGCTCTGATTTTAGTTACTTCTTTTTTTCTGCTGGCTTTGGGTTTAGTTCTTATTTTTCTAGTTCCT
TTAGGTGTGACATTACATTGTTTTAGGTTGTTAACTTGAGATCTTAATGTAGGTGTTTAGAGCTGTAAAC
TTTCCTCTTAACATGGCAAAGAATTTTTTCATATCTGCCTTTGTTTACCCAAAAGCCATTCAAGAGCAAG
TTGTTTAGTTTCCATGTATTTTTGTGGTTTTGAGAATTCCTCCTGGTATTCTTATTTTTATTGCACTGTG
GTCTGAGAAGATACTTGGTGTGATTTCAATCTTTTTTTTTTTTTTTTTTTTGTCTTTGTGCTGTTTTGAT
GTAGGCATCTATTGCTATAAACACTCCTCTTAGCACTGCCTTTGCTGTATCCCATAGGTTTTGGTATGTT
GTATTTTCATTTTCATTTGTTTCAAGACATTTTTGCATTTCTTTTTTAATTTCTTCACTGACCCAGTGGT
CATTCAGGAGCATGCTGCTTAATTTCCATGTATTTGTGCAGTTTCCAAAGTTTCTCTTAATCTTGATTTT
TAGCTTTTGTCCATTGTGGTCTAAGATACTTGATATGATTTTGATTTTAAAAAATTTGTTGAAACTTGTT
TTGTGGCCTAACATATGGTCTATCCTGGAGAATGTTCTGTGTGCTAAAGAATGTGTATTCTGCAGCTGTT
ACATGAAATGTTCTATAAATGTCTTTAGGTCTCTTTGGTCTGTAGTGTAGAACAAACTGGATGTTTCTTT
GTTGATTTTCTGTCTAAATCACTTGTCCAGTGCTCAAAGTGGGGTACCGAAGTCCCCAATTATTATTGTA
TTGGAGTCTGTCTCTCTTTAGCTCTAATAATATTTGCTTCATATGTCTGTGTGCTCTGGTGTTGGATGTA
TGTATATTTACAGCTGTAATATCTTGTTGCTGAATTGATCCCTTTTCATTATATAATGACCTTCTTTGTC
AGTTTTTATGTTTATATGACTTAAAGTCTATTTTGTGTGATATAGGAATAACTACTCCTGCATGTTTTTG
GTTTCCCATCTCTTCATTTTCAGTTTATGCGTGTCTTTACAGATGAAGTGAGCTTCTTTTAGGTAGCACA
TAGTTAGGTCTTGCTTTTTATAGAGATATTCAGCCATTCTATATCTTTTAATTGGGGAATTTAAACCATT
TACATTCAGGGTTGTTATAGATAGGTGAGGACTTAACTCTTGTCGTTTTGTTAATTGCTTTCTGGTGGTC
TTGTATATCCTTTGTTCCTTTCTTCTTCCTTTATTGTTTACCTGTGTAATTTGGTCTCTCTCTCTTTTTT
TTTTTTAGTGATAATGTTTGATGCCTTTCTCTTTCTCATTTGTGTTTCTCCTCCACGAGTGAGTCTTATA
CTTTTTGTGTGTTTTCACGATAGTAGATATCATCCTTTTGCCTCATGATGTAGGAATCCCATATGCATTT
TTTGTAGGGCTGGTCTAGTGATGATGAATTATCTCAGTTTTTGCTTGTCTGGGAAAAACTTTATTTCTCC
TTCATTTTTGAAGGATGGCTCTGTTGGGTATAGTATTCTTAGACACCAGTTTTATTTGTTTTCTTTTAAC
ACTTTGAATATATCGCCCCATTCTCTCCTGGTCTGTAAGGTTTCTGCTGAGAAATCTGCTGTTAGTCTGA
TGGCAATATCCTTATATGTGAGGAACTCACAGAACATCTGACATTTTTCTCTTGCTGTTTCAGAGTTCTT
TGTCTTTGACTTTTGACAGTTTGACTATAATGTGCCTTAGAGAAGACCTTTTTAGGTTGAATCTATTTGG
GGATATTTGAGCTTCCTTTATTTGGATATCTATATCTTTTGCAAGAATTGGGAAGTTTTCAGCTATTATT
TTGTTAAATTGGTTTTCTGTACCTATGCCATCTTTTCTCTTGCTGGAATAGCAAGAATTTGGATATTTGA
TCACTTTATGGTCTCCCATATGCCATGTAGGCTTTCTTTTTTCTTTTTTATTCTTTATTCTTTTGTTGTT
TTTGTTGTTGTCTCATGGGGTTATTTCAAAAGGTCCATCTTCAAGTTCAGAAATTCTCGTACTTGTTTTT
ACTCTGTTGTTGAAACTCAATTGCATTTTTAAATTTCATTTATTGAATTCTTCAGTTTCAGGATTTCTGT
TTGGCTCTTTTTAATGATATCCATCTCTGTTGAATTTCTCTTTCAGAACATAAATTGTTTTCCCGATTTA
TTTTTATTATCTGTGTTTTCTTCTATCTCACTGAGTTTATTTAATAACATTATTTTGAATTCTTTTATAG
GCATTTCATACATTTCCTTTTCTTTGGGATCTGTTACCAGAGAATTATTGTGTTCCTTTGAGGTGTCCTT
TTTCTTTCCTTTTTCATTTTTCTTGTGTCCTTGTGTTGATTTCTGTGTATCTGGTATAATAGTCACTTCT
TCCAATTATATGGATTGGCTTTCATAGGGAAAATTTTTTTCATATAGGTGTATCTATAGTGTTGGTTGGG
TAGGGTGCTTTTGATTTGATTCTCAGTGGGTGCAAGAGTGTAGTTTCTATGATTTTTCCAGCCATAATCA
GTATCAGTGGTTTCTGTGAGTTTTTCAGTGACTTGGACTGTGATTGTCAGTGGAGGCTATAGCAAGGCTC
TGCTGGGGTAGGGACACCAGGCAGACTGGTCATCACGTACCAGACAGTGATGCCAGTGGCAGGCAGGATG
GGCTTGTCCTCAGGCTCCCAGATGGCATGCACAGGCACTGATAGTAGCAGGCAGGGCAGATCAATCCCCA
GGCCCGTGGGTGACATGCACAGGCACCAGTGGTGGCAGTAGTGGGTGAGGCAGGCCTCTCCAAGGTCCCT
GGATGATGTCCATAGGTGCTGGCAGTGATCACGGTAGGTTGATTCTAAGTCACCTAGTGGTGGCAGGAGG
GGCTAGGCTGATCACCAGTGCCTCAGACAATGTGCCTGGTAGTAGTGATGGGCACAGCAGGCCTGTCCTC
AGGTCCCTAAGTGGTGTGTGTGGACTCCCAGTGGTAAGTTTTCTTTTAACCTTAAGCTTCTCACTGCAGT
TTAAAAAACTTATCTGAGTTGTGCCTCTGACCATATTGTGCTTTGGGTACAGAAGCAAAAGTGTTGAGCA
AAAGCTAAGTTGATCCATGTCATGGTCACAGCATTTTGGGGGGACTTTTTTTGGGTTGTTTGCCTCATCA
CAGCAGTGTGAAGCTAGTTGTTTGCCTACATCCTATCTTGAGTTTTCTACTCTTAAGAAGCAAAAGTACA
AAGGTTCTGTCACACTAAGAACAGGTGCTTTTTGAAAGTATTTAAGCCTTACTGAGAGCTGTCTTGTGGC
CATTTCTGGAAACCTCTTTGAATCTAATATATCTGTGTCCACTTCTGTTTCTCAGCAATATCTGGCATCT
CCTGAAATGCCAGGACATCAGTAGGTTTACAATAGTAAGGTAGACTACAGTAAGTTTCTGACAGAAAGGT
GGGCAGAAATAGTTGAAATAAATCTGGCTGAAGGATGAATTTCTTGAAGCAGCTCTTACAATTGTATTAT
CACTTTCCTGCATGATAGTCTGCTTTCTTCCAAGGCTTTTTCCTTTAGCTAGATGAAAATGTGATGTATA
GGCTTGTCTGGATCCTTCCTGAAAGCTTCCTTAATATGTTACAGAGGCGCTTGTTGGCTGTAGCAGCCTG
GCTCCACAAACCAGACTTAAAACATCAATCTTGTGCTTAACCAAACAACTTGGGCTTTAGGTTGGCTATG
AGAGGAAGATAAGATATTCTGTGGAATAAGTAATCATCTTAATATGAGCATTAAAAGTAAAAGCTCAGGC
CGGGCACGGTGGCTCACACCTGTAATCCCAGCACTTTGGGAGGCTGAGGCTGGCCAGATCACCTGAGGTC
AGGAGTTCAAAACCAGCCTGGCCTACATGGCAAAACCCCATCTCTACTAAAAATAAAAATTAGCCGGGCA
TGGTGGCGCATGCCTGTAATCCCAGCTACTCGGGAGGCTGAGGCAGGAGAATTGCTTGAACCCGAGACGT
GGAGGTTGCAGTGAGCCATGATCATGCCACTGCACTCCAGCCTGGGCAACAAGAATGAAACTCCATCTCA
AAAAAAAAAAAAAAAAAAAAAAAAGCTCAGCAATCCTATTCTTTGCTTCTGGTAATTTATTAATTTACAT
TAATGTCATTTAATTATTTACTTCAGGTAGATTTACATCTGCTATATTTTTACATGAAAAAGATGAAAAG
CCACTTGAACATATCTTCTGTTTTTTTAATGATCTGTTCTTCTTTATAGGGGATTTTTGACATCTTGTGA
AGCAGAACTACAGGAGCTCATGAAACAGATTGACATAATGGTGGCTCATAAAAAATCTGAATGGGAAGGA
CGTACACATGCTCTAGAAACTTGCTTGAAAATCCGTGAACAGGAACTTAAGAGTCTTAGGAGTCAGTTGG
ATGTGACACATAAGGAGGTAAAGCAATTTATTTGTTCTTCTTTTTGACATTTTTATCTTGTCTTTTATAT
TAAATGTGTTTCTTAAGTATTTGTTGAAGAAAGAATTTGTATACCAAGTTAGTTTATTGTTTTTTATTAT
AATTCTGCTGTTTTATTTTAGAAGAAAAATTCATTGTGAAAATGTATAAGAATGTTATCATTTATTTCAA
GGATCATTTTACCTTGGTTACATATAATATATACATAACTTCTTTAAGAAAATGTAAAAGGGTACCACAC
ATTGTTTGTTTAACCTTCATCATATTACATGTTTATATTAACATTCACTGAGTTTCATGGAAATTGCTTT
TGGCTTCACTGTATGGCTGGTATTACAAAAGAAGTTCCAGATTAAAGAATTTGCCTCTTTTGGTTAGATT
TAAAACAAAGTTAAGAGAATCTTTAAAAAAAAACAAAACAAAACACGTTTACCATGTTGAAAATTAGAGG
AGAAGAAAGAAAAGAAGAAAATAAAAATTACTTGCAGTCCCACCACTCATTGATATTGGTAATTTGGGGG
AAAAGTTTAATAATATTTGCTGGTTGCCTTCTCATAAATTTAGCTGGTAAATTAGGAGGAAGTACACAGA
ATATTGGAAGGGAAGTAGAAAGCATTTATGTGTGAGAGTTGCCTGTTAATTTTTGGTATGGATGTCAATC
TCAGAAGGATTGGAAGAGTTAGAGTTATAGGGGAGCACTGGTCCCATTTCTCACTCTGAAGACCTTTTCT
ACCAGTAGGGTCACCATTCATTAAGGCTGTCATCAGCTTATTCAGGTACACTTCTGTGAGTGGTACACAC
TGAGATTGTGATTTTGTTGAGAAAACTTATTAAGAATAGTTTAGAAATGTTTCTCGTAACAAAGAGTTTG
ACTTGTTAAAAATGTTATTATGCTTACCCAGAAAATTTCTCCTTGGTTGGCCATTTGCAACTTGGGTCAA
AGTAGTGCGTGGCAACACTGGCTGTATCAAAGTTTTCTGCTAATGCTGTTTGCTAAATTGCAATTAATTT
AAAATTTGACTAAAACTTTGAGAATATATTTTAAAATTACACACCATGCGAAAATACAGAAGGTGATGCA
AAAGTACAGTGAATGGTGATCTTGTTTGATTTTTTAAAAATCATAATTTAGGCAGGGTCAATGTTCCTTT
TATGGTAGGCTACTTAGCTAGCACTTTGTTTCTCCTAGGGCAAAACAAAATTCTGTTTAGCAAGTTGAAA
TGAAAGTGGTTTTCTGGTTGATAAATAGATTGTTTATTTTGTTAGAAACTCATTAAAGAATTATGTGTGA
TTATGATAATATTTAGTCACTGTAATGCTAAATTTGAACTTATTCCTGTGGCTTAGTTTTGACGCCACGG
GCATTGTTGCCGTTACTTAATGCCCTTGACATCAAAATTTCTCTCAAAGAGAAATTAAGAGAGATTTAAG
GGATTAAATGGATAGATTCCATGGTAAGGATATGAAGATGTTAAGTTTGGCCTGGGGACATACAAGAAAT
TTAGAATTGCAGCTCTCTGGTTAATTTGCTACTTCATTGATTGGGCTGCTGAAAGTTGATTAAGTTTCTA
GAAGTAGGTTTCTGAAGATAATCTAGTAATGGGAATCTATTCCATAGAATTTGTGTGTGAGATTGTGTTG
TATTACAACACTTCTTTCTTGCTTTTTTTTTTTTTAAGGAACAAATGTGTTTATTATTGTGCTGAAAAAT
TATGACCACCAACAACCAATGAAGGGCAGGAAGGAACAACACTATGAGATCATGAAAATATTTTGCTTCT
TGTATTAGTCTGTTCTCACATTGTGATAAAGAAATCTGAGGCTGGGTAATTTATAAAGGAAAGAGGTTTA
ATTGGCTCACAGTTCTGTAGAAACATAGTTGCTTCTGGGGAGGCCCCAGGAAACTTTCAGTTATGGCGGA
AGGCAAAGGGAAAGCAGGCCTATCTTCACGTGGTGGAGCAGGAGAGAGAGAGTGAAGTGGGAGGTGCTAC
ACACTTCTAAACAACCAGATCTCAGGAAAACTCACTACAACGAGAACAGCACCAGTGGGGACATCTGCCC
CCATGATCCAGTCACTTCCCACTAGGCCCCACTTCTAACATTGGGGATTATAGTTCCACATGAGATTTGA
GCAGGGACACAGACCCAAACCATATCACTTGTCAACAAACTTTCTTCTCTTGCTTTAACATTTTTGACGA
TTCTTTCCCAAACCTATTAAGTAATGATGATGGTTACAAATTTTCCAGTTCTACCACTCTTTCCAGTGCA
TTATTTTTTATATCTTCATTAAAGGAGCAAAGCAAATCCCCTACTTGTAATAATAAGACAATATTGGAGA
CTGCCATTAAAACACAGGAGAAGTAAAAAAACAGATCTGTTCCAAGACACACAGGACTATAAATTTAGGA
AATACACAGAAAATTAAAAGTTAAACTCAATTGGATACATTAAATATGTGCAGCTTTTTGTATGTCAATC
ATACCTAAATAATGTCATTTTGAAAACAAATGGTCCAACCTGTACCACCAACAGGTTTTGAGTATATATA
AATCTAATATGTATTTATGAGTAGGGAGCAACCTTAAACATTTTAAACAGCAGAAATACACAAAGCAATT
ATAGGTGGAAGTAATACAAAGTGCCTTGTGTTTCTATCCTAGAATCAAACTATAGAAGTCTCAGGTTATT
AAGAAAACAAATATTGTTTTGAATATTAAAAATCATGTGTTTTGGAGAGGGAGAGAATTAACAGCCTTTC
TCTGCCTTAGAATACTTTACATTTTGTGAAATTTACAATCAGAATGGAGCATCTCCTAAAGTTGTCAGTA
TGCTAGGGAGGGAAATTGAGCACAGGAGCAAACCACTGTTTTCTTAGTGCTCAGCTGAAGAAATTTTCAC
TTAAAAGAGCTGTAGCTGTTGAAGTCATTTCCATTTTAGAAGCGTACTATAAATGACTTTTTCTATGTAT
TAACTAGAATTAATGGTAATTTGTGAAATGTTTTATCATTTTTAAAATTAGAGTCTATATCTCAGCCAGT
GAAGCAGCAGGCACTGCTGCATGTCATAGTCTAACACCTGCAATCCACCAGTCTGCATGGACTGTAAGCA
GGTATGGAGAGAACCGGAGTTCACCTATCAGCAACACCACCATGGAATCGACTGTTGAGCCTTTCTACTA
TTAATTACCATATATGTGCCTTTTTTCTGGTATTTTATTCCCACTGGATGCTTTTTATTAAGACCTTGTA
CCTGTGAACACATTTGGGGCACAACTTACTGTCTTCAAGGTGCAGGGGTGGGAAATTGGGAGGCGGGAAG
CAAGAAGAGTGCACTCACTCTCCCTTCTTGCATTTCTGGAGCCCAGGCTTGTCTAGCATGAAACTGGAGG
TGAACCCAACCATCTGTATCTGCAGGGAGACTTCTCCCATGGTCTTCTCTTGTGGAGATTCCTCTGCCTC
TGGTGTCCCACCCTGGCTGCAGTTCTGGCCCTGGAGGCCAGCAAAGCACCTGGGGTAGAAGTTTTCTTAC
AGCTGTATTAACTCCTTCAAGGAGAGCAGCAGACTCTTCCAAGGCAGGCAGCACCCCCCCAAGAGGGAAG
AGCCATCTCAGGAGGATGTCTCCTCAGAACTTTTCAGGGACGTTTACCCCTTGTCCACCTTTGATTTTTT
TTTTTTGTAATGATTTCTGAATTCTGGCCTCTTTTGCTGTGCTACGAATGTCCTTTTATTTTGGGTAGGT
GAAGGCACACGGGTGCTTATGTGCATCTAATTTCTGGCCTTTGGTGCTCAGCTTTAATTGTTGGCACCAG
GCACACAGAATGTCCTGGCGAATCAGGTTAATGGGTAGCAGTTTAGAGGGTAATGGAGGGATTGATATCT
TCTTCTGAGGTTTTAGGTTGTTGTCACTGAACTCTGCCTTCCTCTTTGGGCAGAGCACTTTGTCACCTTT
TCATTTGGTCCCCTTTGCTGATGTTCCTGGCAAAGCAATTGTGCTTGGTTAATTAGAAGTTTACTAATCC
TCTTCCCTTGACTTCTCTGTGGATTTCCACTCTTTGCCTTTTTGACTTCTCCATACTCGGAAAAGAAGTG
GAGGTTGACGAAATTTTTCCAGAATCAAAATACTTCTTTCAAGTATGTGTTATAACACATTCATAATCCA
ATTTGAGCAGTATATGAAATAAGAACTAGAAAATAGGGCTTTATGTTTTTGTCATTGTATTAGTTCATTC
TCTCATTGCAATAAGGAACTACCTGAGACAGGGTAAGCTATGCAGAAAAGAGGTTTAATTGACTCACAGT
TCCACAGGCTGTACAGGAAGCATGGCTGGTGAGGCCTCAGGAAACTTAATCATGGCAGAAAGTGAAGAGG
AAGCAGGCACATTTTCACATGGCGGAGGAGGAGACGGAGCAAAGGGGGAAGTGCTACATACCTTTAAAGA
ACCAGATCTCATGAGAATTCACTATCACAAGAACAGCAAGGGGGAAGTCTGCCCCCATGAGCCAGTCACC
TCCCACCAGGCCCCTTCTCCAACATTGAAGACTACAGTTCAACATGAGATTTGGGTAGGGACACAGCCAA
ACCACATCAGTCATTCTTACTTGTTTGAAAGGTACAGACCTAGAAGCACTTAGTCCTAGGGTGTAAACCA
CTGCTAATCAAATGACAGGTCCATGATGAGATTAAAAGCTTGCACCAGAATGTAAATCATTGTAGAGAGC
AAAACCCCTCATCTTTCATAGTACCAAGTCATTAAACAGCTAAAACTGTTAACAGCTAAAATCAGTGTAT
ATCCATCTTTTGCTGAGAAAATTTTGGTTGTAAGAATGTCTGTTACACTGAACGGTGTGTTTCAAGACAT
ACTTGATTTACACTCTGCCTCAGGCTTCTTGTGCTGGATTGCTGTCTGTGAATAACTTCAGCCCACCTGC
AAATCATACTTGGTGGATCACTAATATAAGCAACTTGTTTTGTAAGTTAAGGCTTTATGTGTTTCTGGTT
ACAGTGTTGGCACTGGGTTGGTTTTAATGGACATTCCTGCTGTTACTATCTTCTAGCTCCCTACAGGGCA
CGTTGACTTTGTGAAATGAGTAAGAAATTTAGAGTCCTGGCCCTGCCTCTTTTCAGCTGAATGGCCTTGG
ACAAGTAACTTCCATCATTATTAGCATTTAATGTTCCTAAACTTTTCTTCACCTTTATATTTATACTTGT
CACAGTAAATAAAAAGAAAAATGATTCTACAAGGCTCATAAGAAAAAATAGTAATCTTCCCCACCCCTCT
GAACCTAGGATTCTTACTTCTGTGAGGAGTAATTTCAAGCCTTTAACTCTTTCTTTTGGCATTTTCCCTA
TGTTTCCAAGGAAGCTTTTTTACTGCTGTATTTTCATTTATAGTTTTAGCTGTTTAATGACTTGGTACTA
TGAAAGATGTGGGGTTTTGCTCTCTTACATACAATTATCATCCCCTCATCCTTCCAGGAGTGTATATCTT
AATTTTTTGTTAGATTAATATTCAGTATTTACATCATTAAATGGTAAATATTATTCACAGTTGAGTCAGG
TAGTGTAATTATAAGCAGACTTGTTTTTTGTACTGCTTTTTTGTTTTCCTGGAAATTACTAATTGTCTCA
CTTTCATTTGGTTTTTTAAACATACCTACCATTGATTCTTTCTGAAATCTCTGACACAGGTGTAAATCTT
CTGCCATTGTGTTTAAACAAGGCAATTAATCTGTCCATTTTCTTTTCTTTCATTTCTTTTCCCTGTGAAG
ACATCTCTCCTGGAGCTTGCCATTCTCCACCGTGGATAGGTTGCTTTCAAGGATAGGTGTATAGCTGTCA
TCATGAGAACTCCCTTCATCATTATTCTGGAGGTTTTCTGTGCTCCTCTATTTGTGCTTCTTTGCATTCT
CTGTCACCATATTTATATGTTTACATTTCTATTTTGATAGAGCATAGTTTTGAGATCTTGTATATCTGAA
AATAATCTTTATTCTATTTTCAGGCCTGATTGAAAGTTTGACTGGCTGTAGAAATCAGCTTTAGAAGTAA
ATTTCCCTCAGGACGTTTACAGTTAAATATGAATTATTTCAGACTTTAATATTTGATAATGGCAGATTAG
GTAATTTGGGCCAGTCCACTCACCGAGGACAAATAGGTAAGTTAGATTTAAAAAAAATTTTGCTTGAAGT
AAAATGCTATCAGGATGGTGAACAATTACCAGGCTGGGATCTGGGCTGGGCAAGCGGTTCAGGGAGGTGT
GTCCAGGGTTTGGGGCCATTATTAACTGAAAATAAAGAAGGCAGCCGAGAGGATAAAAGGTTGAGTGGTG
TTTTTGATAACTTTAAGGGGCTAGGGGTCCATGATGGCTCTGAACGCCTCCTCAGTTTGGCTAAAGACCC
TGAAAGGCTGCACCCTAGGAGTAAGGATGGACCAGAACAGGCCCTCTTGAAAAAACCACTCAGCTTCAAA
CATCTAAATCCCTGAAATCAGTTTTAAGTTAAACTTAAGAGTGCTACTGTCCCCAGGCATCTCCAAAGTG
CTAGGGCTTCCAGGAACCTGGAAGTAGCAAATATAAATTTTCTTCAAAGGAAAAATTACCATTCTGGGTT
TCAGATTATTCAAATAATTTTGCAAATAATGCTTGGTACTTGCTCAGAAAATAACCTGAGACAACAAAGA
CCAGGAGACAAGACAACACAAAGGAAAAATAGTAGAAGCAGCAGAAGATAGAAATAGACCCATGATAAAT
CTAGATATTTGTCATTATCATGCACACAGTTTACAATAACAATGTTTATTGCATTCAAGGCACTAAAAGA
TAAGATTGAGCATTTCGAAAAAGAATTAAAAACTGTAAAAAAAATGAGTGGAAATTTTAGAATTGAAAAA
TAGAATGACTGAAATTAAGGTCTTAAAGATTAAGGGATAATTAGTGAACTAGATGATGTGACAGAATAAA
ATGTCTAGAATAAAGCACAGGCAAAGTGATAGAAAATATAGATGAAAGGATATGAGAAAGAGAAGACAAC
AAGAAAGTCTAATATAAGTGTTTTTGGAATCCCTGAAGGAGAAAAAAAATGAGAATGGGCACAAAAGATA
TTTGGAGAGATACTGGCTGAGATTTTGCCAAAAGTGAGGAAGGTCAAGTATTTAAGAAGTCCAAGAATCT
CAAGCAAGATAAGTAAAAAGAAACAGTTGCCATGGTTGAAAACTAAAGACAAAGGGAGGAATCTGAAAAG
CAATCAAAGAAAAAAAAGATTATCTACAAATAGCTCTTAACAGTTGACTTGTCAGCCAAAAAAGTGAAAG
CCAGAAAACAATAAAATGTCTACAAAGAAAGAACATGACTTTTTCCTCTACCAAACGAGAATTCTGTATC
CAGAATAAAGGTAAAATAAAGACATTTTCAGATGAACAAAAATTGAGAGAACTGGTTACAAGCAGACCTG
CACTAAGGAAATATTAAAAAATGTTCTTTAGGTAGAAAGACAGTGATCCCAGATTGACCCTCAGAGCTGC
AGGAAGAAATGAAAAGCAGTAGTAAATGTATGGGTAAATCTAAATGAATATTGATTGTATAAAACAACAA
TAATAGATGGAGTTTTGCTATGTTGCCCAGGCTGGACTCGAACTCCTGGGCTTAAGCAATCCTACCTCAG
CCTCCCAAGTACCTGGGACTACAGGTGCATGTCACTGTGCCCAGCTAACGGTAATTGCCTTTTGAGTTTA
ACGTATATGTAAAATTATGGTGGCAATGGTGTTATATTTAAGTCAGGTTGGGATAAATGGAATTGAAGTA
TTTATAGCTAACATTAGACCTTGTTAAGTCACAAATCCATGTTTTTATCTTGAGGGAACCATTAAAAGTA
TATATTAGTAGAGAATATCTTCCATGCTAATAGAGAAAAATGGAATGATAAAAAAAGTAATCAGTCCCAA
AGAAGGCAAAAAAGAAGAGAAAAAGGAACAGAGCTTATTAAGTACAAATATTATGTAACAAGATGAAAGC
TTTAAATCAAAATAGGTTTGTTAAATGTAAATGCAGTAACTGGCTTAATTAAAAGAAAAACTGTCCAACT
GGATCAGATAAACAAATGCAATTATTATTTAATGTTTATAGAAATATAAAAACATTGAAATTAAAAGGTA
AAATATATAACATGCAAACAGTTTGACTAGAAAGATGGTATAACTATATTCTTACCAGAAAAAGTATGAT
TGGAGCAGAAGCCGAAAGCATTACTCAAGGTGAAGAAAAACACTTCATAATGAGAAAATGTGAAATTTCA
GGATATGTAACTTTAGTAGACATTCTTTGTGCCCTGTAGTATGTCCTCTTTGCCTCCTGATTTCCTTTTA
GCTATTAAATATAGGTCCTTATGCGCTGCCAACATCTACTATAACAGGAGTTTCCAATCTTTTGGCTTCC
CTGGGCCAGATTGGAAGAAGAATTGTTTTGGGCCACACATAAAATACACTAACACTAATGCTGGCTGATA
AGCTTAAAAAAAAAATCTTGTAATGTTTTAAGGAAGTTCACACATTTGTGTAGGGCTACCTTCAAAGCTG
TATTGGGCCACATATGGCCTGTGGCTTACAGGTTGGACAAGCTTGTAGTATACTTTGTTTGAAGGGGTGG
GTTGCCCCTCCACACCTGTGGGTGTTTCTCGTTAGGTGGAATGAGAGACTTGGAAAAGAAAGAGACACAA
AGTGTAGAGAAAGAAAAAAGGGGGCCCAGGGGACCGGCGTTCAGCATATGGAGGATCCCGCTGGCCTCTG
AGTTCCCTTAGTATTTATTGATCATTCTTGGGTGTTTCTCGGAGAGGGGGATGTGGCAGGGTCATAGGAT
AATAGTGGAGAGAAGGTCAGCAGACAAACATGTGAACAAAGGTCTCTGCATCATGAACAAGGTAAATAAT
TAAGTGCTGTGCTTTAGATATGCATACACATAAACATCTCAGTGCCTTAAAGAGCAGTGTTGCTGCCTGC
ATGTCCCACCTCCAGCCCTGAGGTGGTTTTCCCCTATCTCAGTTGATGGAACATACAATCGGGTTTTATA
CCGAGACATTCCATTGTCCAGGGATGGGCAGGAGACAGATGCCTTCCTCTTGTCTCAACTGCAAAGAGGT
GTTCCTTCCTCTTTTACTAATCCTCCTCAGCACAGACCCTTTACGGGTGTCGGGCTGGGGGACGGTCAGG
TCTTACCCTTCCCATGAGGCCGAATTTCAGACTATCACATGGGGAGAAACCTTGGACAATACCTGGCTTT
CCTAGGCAGAGGTCCCTGCGGCATTTGTGTCCCTGGGTACTTGAGATTAGGGAGTGGTGATGACTCTTAA
GGAGCATGCTACCTTCAAGCATTTGTTTAACAAAACACATCTTGCACAGCCCTTAATCCATTTAACCCTG
AGTTGACACAGCACATGTTTCAGGGAGCACAGGGTTGGGGGTAAGGTTACAGATTAACAGCATCTCAAGG
CAGAAGAATTTTTCTTAGTACAGAACAAAATGGAGTCTCTTATTTCTACTTCTTTCTGCACAGACACAGT
AACAATCTGATCTCTCTTTCCTTTCCCCATACTTTGTTTCTCTCTCTGGGGCCTTGGAAAGCTGCTTAGA
TGAAATCTGGAAGTGTTGAGGAGTTAATACTCTTAGATCTTAGGTCAAGGTTCAAGTAGTGGAGGGACTA
GGTGAATTCTCCAACTTCCCATTCCTCAGAGAGACGATTCTGAAAGATTCCATCTGGTTCCTTAGTGGAT
TCCCAGTGGAATTCATCACCAGCTGCTTACTTCAGTAATATGCTTAATATGCTCAATAGCACGTCCTTTA
TTAGCTTTCCTTCCTTCTCTGCCTTGCTCTCTGTGTCCTCACTGTGCTTTCTGGGGTCACCTCCAGTTGC
CCTGAAGTCCATGTCTCAGCCCACACTTTGCTGGGGGACTCAAACCAAGATGAAAATGTCTGCGTATCTA
ATGTGTCTAGTTAAATAGTTTTAAAAGAAAATCCTCTTTCTGTCTTATCTGTATGTGAAAAGTGACAGAA
CTAGGAAAAAACAGATGAAGCCACAATCATAGGGGAGAATTTTAACACATTTTTCTTAGTAACTGATGGA
ATAAGAGACAAACATAAGAATATAGATTTTAACAGCATGATCAGCGAACTTGACTTAATTTTTATATATA
GAATATTATACAACAGAATTTCAGAGTACACATTCTTATTAGCACAAAAAATACCCTTACCAAAATTGAC
CATATGCTGGGCCATAAAGTAAATCTTGATACAGTTCAAAAGCTTGACGTCACAGGTGTGGTCTCTGAAC
ATAGTGGAAATAAGTTAGAAAAAGATAACTTGAAAATCTGCATACATTTGGAAATTAGGACATGAAGTTC
TGAATAATTCATGGATCAGTGAGGAAATCACAATGGAAATTAGAAAATACTTTGAATTCAAAATGAAAAT
ACAACATCAGACATACAGAATGCAGATAAAATATCTCAGAGGGACATTTATAGCCATAAGAGCATATATT
AGAAAAGAGGAAGGGCTGAAAATCATTGAGGTATTGATTCATCTCAAGAGGTTGGGAAAAGAACTTGCTA
AATTTGAAGAATGTAAAAAGAAGGACATTTTTTAAAAATGAAAGTAAAAGCCTGGAAGTGATATGACATG
AGAAAACTTCCAAAAGAAAACTGATGTAACTGTATTGGCATGAGACAAAGTAAACATCAAAGCCAGAAGC
AATACTAAAGATCTAGGGACACTTAAAAATTATGAATGGGTCAGTTAACCAGGTTGCTTTAATAATTCAA
TTCTATAAATACTTAATAATGTAGCCTTAAAATATACAAAGAAAAATGGGCAGAATCAAGGGAAAAATTA
AGTACATGGTAATCATGGAAGACTTAACAAGTAGCTCTCAATTAATACAACAAGCAGAGAAAACCTCAGT
TCAGGTGAAGATTTGATCAAGATTAACAAACTTAACCTAGTTAATAAACTTGACCTAATTAATCTAAATA
GAACATTGTACCTAGTAACTGCTGAATACACATCCAAGTGAATATGGACCATTTACCACTACTGCCGTAA
AGCATGTCTCAACATATTTCAGAGGATTAGAATCATGTTTTTTGACCAAGGTGGATTTAATCTAGAACTC
AGAACAATGAAAGACATCCAGAAAAAATCCTCAAAAGCTTGGAAATTAAGCAATACACTTCTAAATAACC
CATGAATCAAAAGAAATCAAAATGAAAATTAGAAAATATTTTGGACTGAATAATAATGAATATGGTACGT
ATCAAAACTTGTGGAATGCAGCTAATGTGGTGCCTAGAATGTTAGCTATGTGTGTATACATATAGCTACA
TTTTATATACAGCTAAAATTATATATATTAGAAAGGCTGAAAGTGATCTAAGTAGCCATTTCAAGAAAAA
CAAAAAAACCCCAGCAAATTAAAACCAAAGAAATGAGAAAGAAAAGATGTAATAAAGATAAGAACAGAAA
CTAATAAAATAGGAAACAAATGTATAAGAGGATGAACAAAGCTATTCTCTTCGAAAAAAATTAATTAAAT
TGATAACCTTGGCAAGACTAATTAAGAAAAAAGGGGGGAAAATAACTAATGTTAGCAATTAAAAAGAGAA
ACCATAATAGAGCCTCTGAATAGTAAAGGGGTTATAAAAAGATACTATGAATGACCATGTACCCACCCCC
CCACCACAGTAAAACATTTAGATATAATAAAAAATCCCTAGGAAAGCACAGCATACAAGAAAGGTTGAGA
CATGTGAATAGTATCCTATTAAATTGATTTTTTTTTTGTTTTGAGATGGAGTCTCACTCTGTCGCCCAGG
CAGGAGTGCAGTTGCGTGATCTCAGCTCACTGCAACCTCTGCCTCCTGGGTTCAAGCGATTCTCCTGCCT
CAGCCTCTCGAGTAGCTGGGTCTGCAGGTGCGCACCACCATGCCCGGCTAATTTTTGCTTTTTTTTTTTT
CTTAAGTAGAAATGGGGTTTCACCATGTTGGTCAGGCTGGTCTCAAACTTCTGACCTCAGGTGATCTGCC
CGCCTCAGCCACCCAAAGTGCTGGGATTACAGGCGTGAGCCACCACGCCCGGCTGAATTGAATTTGTTAT
TGAAAAACCTGCTCATAGATAAATCTCTTGGGCCCAGTAGACTTCATTGGTGAATTCTACCAGACATTTT
AAGAAATAAGTAACATGAATTTTATATAAAATATTCCAGAGAACAGAAGAAAAGATAAGATTTTCCTAAC
TCCTTTTATGAAGCATCATAATCTTTATTTCAAAACTTAACAAGGATGTAGCAGTTAAGGAAAATTACAG
GCCAGTCTTATGTGAATGTTAATATAAAACTGTTAGTCAAAATATTAACAAACTAAATCCAGCAATATTT
AGAAATTACATACTAACTTAGGTTTATTCTAGTCATTCTGGGTTGTTTCAACATTCATAAATCAACATAA
TTCATCATATTATCAGAGGAAAAAACACCATAGTTATCTCAATCCAGGAAAAGAATTTAATGAAATTCAG
TTATCCATTTGTGATAAAAATATTAAAATCCCTTAGAAAATTAGAAATGTTAAGGGAATTTCCTTAATCT
AGTAAAATGGTATATAAGAAACATAGCAAGTAGCAATTAATGTAACCCATTGTTAGAAGCTTTCCTTCAG
AGATTGTGAATGAGACAAGGATGCCCAGTATCACCACTTTTTTTGTTGTTGTTATTGTAGTGGAGGTGTA
GCTAGTGCAATAAAGCAAGAAAAATAAATAAAAGACATAAGGATTAGAGAGGAAGAAATACCACTGCAGT
AATTAGCACATGCTTTGTTGGTGTACACAGAAAACCCAAAAAATCTATGTAGACAGAATTAACAAATGAA
ATAGTAGATTTGCTGAGTTAAAGGTTAATTTACAAAAATTTATTGATTCTTTGTACCAGCAAAAAGCAAT
TAAAAATGAAGTTAAAAAAGATACCCTTGACAAGAGTATCAAAAAAGCACCAAATAGAGTAAATCCAATG
AAAGAGGTATAAGACCTCTGCACAGAAAGCTCTAAATTATGGAGAGAAATTAAAACTGATATAAATGGAA
GAATAGATGTTCATGGATAGAAAGACTCAATATTTTAAAGGTATTGGTTTCTCCAATTTTGATCTATTCA
GTATTATTCTAATTAAAACTCCAGTATATTTTAAAGTGAAAATCAACAAGGTTGATTCTCAAATTTATAT
GGCAGTAGCCAAAACAATCTTGATGAAGAGAAATTAAGGTGGAGCACTTAGGGTACTAGAAAACAAGATT
TATTAAAAACTATATTAAGACAGTGTGGTATTGCAAGGATAGAAAAACAGACCAATGGAACAGAATAAAG
AGTCCCGTAACAGAGATCCCACATATGTGAACATTTGATTCATGACAAGATGACACTGCAAGATGGTGGG
GAGTCTTTTCAATACATGTCACTGGATCAACTGAGTAACAATATGGGGGAAAAAGAAAGATTTTGACTTC
TAGTTTTATACCATATACCAAATTCAATTCTAGATGGATTACGAATTTAAATGTGAAATGCTGAAGATTT
TCTAGAAGATGACATAAGAGAAAATCTTAGTGCCTTGGAGTAGGGAAAGATTTCTTAAAAAATAGTGATA
AAAGGTTACTAATTTAGACTATATTAAAATAATGAACATTTCATATATTTACAACATACAACCAACAAAA
GATAAAGAACATCTATAAATTAATAAGTTAACTGAGTAGAAAAATGGGCAAAAGACAAATAGTCATTTCA
CAAAGGAAGATATCAAAATGACCAGCGAGCATGTGAAAAGGTGCTTGGTTTCACAGGTTACTAGGAAAAT
GCAAATTACAATCACAAGATAACCCTAAACATACACCAGAAAGGCTAAAAGTTAAAAGACTAATAATACT
AAGTTACTATAGGGGTATGAAACAGCTCAGTATTTTATCAAAAAGAGTGCTGTACTTTTCTGTGCAAATA
TTTACTTTCAATTTAATTTTGTTTTAAAAAGCAACAGTCAATTCCCATTTTCAGTAGTAGAAGATACACA
TTTTGTGTATTTTTTCCCATGGGTATTCTGGATTGTTCCCCCGTTGGGCCTGGAAGTTTGGAAATGGTTG
AACATGACTTAGCATGTTTGCTTTCTGCTTTAAACACTAAAAGTCCTTGTCTTTTCACATTTTACATTTA
TGCAAAGTACATTTGTATTTTTCTCCTTGCTTATTTCTTTTATATTAATATATTCTGTTTACATGCATAA
TTTTTCCCACTTTGTTTTATTCTAGAACTCTTGTAGTAGGAAGTACTAGAAGATCTGTGAATATCTTAGT
CTTGTTACCTTCATTAACAGCTAATATTCTCACTTCTGTTTAATGATCAGTGACTTAGTATTGCATAAGA
ATCAATAGTCCAATTAACTAAATTGAAGCGCTAATTTCTCCCTTTCAGTGTTACTGAAGTTTTATTTTCT
GGGAGGGGGATGTTGAATTCCACATTATTGTTTTTAGTGTTTTTTCAGAATCATTTTTCCAGAATACTTT
TTACCTTTTTGAGGATGGACTTTTTGCAGTGCACTTTAACTGTATAGAGTGTGCAAATGACATTTCAGCT
GGACTTGGGGGAGCTGTTCTGGCATTTTGGTTTGCAAATCAAACATGCACTCTCTTTTAGTCTCTTTTCT
TTGTATTTCTTCTCATTGTTTCTTTTGGATCTGGTACTGTCATCTGCATTTCTTCATTAATTAACAAATT
TGCCTGGAGATAATTTTTACTGCTCCTTGTCTTAAAAACATTTTACTTCCTACCTTTCTCCTTGTCAATA
AACAGGATATATGTAATTTGGGATTTATCTAATCTGTTAGTGATGGATGAAAAGACTCAAAGTGCCAAGA
GATCAGGAAAATTCAGATCAATCCAGGCAGGAGGAATCATTAGGTGTGACTCTCAGAAGGACAGGGATCT
TCAAGTGGGTCACAGGGTCAGACTGGCAAAGCGTTTTCTTCTGGATGTGGGTTCACAAGGGATATAGGTG
ATTCTAGTGAAAGAAGAGAGCCAAATGAGAGTGATTGTGAAGCGTTGTAGTCAGAAATGTGATTGTTTTT
CTTTTTTTAAAATTTAAATTTTATTTTAAATTCAGGGGTACATGTGTAGGTTTGTTATATAGGTAAACTT
GTGTCGTGGGGGTTTATTGTACAGATTATTTCATCACCCAGGTATTAAGCCTAGTACCCCTTAGTTATTT
CTTTCCTGATTCTCTCCCTTTACCCTCCAGTAAGCCTCAGTATGTTGTTCCCCTCTATGTGTCCCATGCG
TTCTCATCACTTAGCTCCCATTTATAAGTGAGAATATGCGGTATTTGGTTTTCTGTTCCTGCAAAAGACG
TGATCTCATTCTTTTTTATGGCTGCATAGTATTCCAATGTACCTCATTTTCTTTAGTCTACCATTGATGG
ACATTTAGGTTGATACTGTGTTTTTGCTCTTGTGAATAGTGCTGCGGTGAACTTACGTGTGTATGTGTCT
TTCTGATGGAACGATTTACATTTTTTTGGGTATATACCTAGTAATGGGATTGCTGGGTTGAATGATATTT
CTGTTTTTATATCTTTAAGGGATCACCATCATCTTCCATAGTGGTTAAACTAATTTACACTTCCACCAAC
AGTGTATAAGCGTTCTCTTTTCTCTGCAACCTTGCCAGCATCTGTTATATTTTGACTTTTAAATAATAGC
CATTCTGACCAATGTGAGATGGTATTTCATTGTGGTTTTGATTTGTGTTTCTCTTAAGATCAATGATGTT
GAGCATTTTTTTATATGATTGTTGGCTGCCTGTATGCCTTCTTTTGAAATGTGTCTGTTCATATCTTTTG
CCCAGTTTTTAGTGGGGTGGTTTGTTTTTTTCTTGTAACTTTTAAGTTCTTTATAGATGCTGGATACTAG
TCTTTTGTCAGATGCATAGTTTGCAAAAGTTTTCTTCCATTCTATATGTTGTTCACTCTGTTGATAGTTT
CATTTGCTGTAAAGAAACTCTTTAATTAGATCCCATTTGTTGACTTGCTTTTGTTGCAGTTGCTTTTGGT
GTCTTTGTCGTGAAATCTTTGTCTGTTTCCATGTCCAGAATGGCATTGCTGGGGTTGTCTTCCAGAGTTT
TTTATAGTTTTGGGCTTTACACTTAAGTCTTTAATCCATCTTGAGTTAATTCTGTATATAATACAAGGAA
GGGGTCCAGTTGCAATCTTCTGCATGTGGCTAGCCAGTTATCCCAGCACCGTTTATTGAATAGGGAGTCC
TTTCCTTATTGCTTGTTTTTGTCAGTTTTGTTGAAGATCAGATAGTTACAGGTGTGCAGCCTTATTTCTG
GGTTCTCTATTCTCTTCCATTGGTCTATGTATCTGTTTTTGTACCAGTGCCATACTGTTTTGGTTACTGT
AGCCCTGTAGTATAGTTTGAAGTTGGGTAGTGTGATGCTTCTAGGTGTGTTCTTTTTCCTTGGGATTGCC
TTAGCTATTCAGGCTCTTTTTTGGTTCCATATGAATTTTAAAATAGTTTTTCCTAGTTCTGTGAAGAATG
TCATTTGGTAGTTTAATAGGAATAGTGTTGAATCTATAAATTGCTTTGGGCAGTATAGCCATTTTAATGA
TACTGATTCTTCCTTCCATGAGCATGGAATGTTTTTCCATTTGTTTGTGTCATCTCTAATTTCTTTGAGC
AGTGTTGTATATTTTTTCTATAGATTTTTCACCTCCCTGGTTAGCTATATTCCTGGGTATTTTATTCTTT
TTGTGACATTTGTGAATGGGATTGCATTCCTGATTTGGCTCTCAGCTTGACTGTTCATGGTGTATAGCAG
TGCTGTGACTTTTGTACATTGATTTTATATCCTGAGACTTTGCTGAAGTTGTTTATCAGCTTAAGGAGCT
TTTGGGCTGAAACTATGGGGTTTTCTAGATAGAGGATCATGTCGTCTACAAACAGGAATAGTTTGACTTC
CTCTCTTCCTATTTGGATGCCCTTTATTTCTTTATTTTGCCTGATTGCTCTGGCCAGGACTTCTAAAACT
ATGTTGAATAGGAGTGGTGAGAGAGGGCATCCTTCTCTTCTGCTGGTTTTCAAGGGTTTCCAGCTTTTGC
CCATTCAGTATGATATTGGCTTTGGGTTTGTCATAGATGGCTCTTACTATTTTGAGGTGCGTTTCTTCAG
TACCTAGTTTATTGAGAGTTTTTAACATGGAAGGGTGTTGAATTTTATCAAAAGCCTTTTCTGCATCTAT
TGAGATAATCATGTGGTTTTTGGCTTTAGTTCTGTTTATGTGATGAATCACATTTATTGATTTGTGTCTG
TCGAGCCAACCTTGCATCCTAGGGATGAAGCCTACTTGACTGTGGTGGATAAGCTTTTTGATGTGCTGCT
GGATTTGATTTGCCATTATTTTGTTGAGGATTTCTGCATCAATGTTCATCAATGATATTGGCCTGAAGTT
CTCTTTTTTTGTTGTGTCTCTGCCAGGTTTTGGTATCAGGTTGATGCACAACCCATAGAATGGGTTAGAG
AGGAGTCCCTCTTCCTCAATTTTTTGGAATAGTTTTAGCAGGAATGGTACCAGCTCTTCTTTCTACATCT
GGTAGAATTCAGCTGTGAATCCATCTGACCCTGGGCTTTTTTTGGTTGGTAAGCTATTTATTACTGATTC
AATTTCAGAACTTGTTATTGGTCTGTTCAGGGGTTCAGTTTCTTCCTGGTTCAGTCTTCGGATGGTGTAT
GTGTCCACGAATTTATCCATTTCTTCTAGATTTTCTAGTTTATGTACATAGAGGTGTTTATAGTATTCTC
TGACGTTTGTTTGTATTTCTGTGGGGTTAGGGGTGATATCTTCCTTATCATTTCTGATTGTGTCTATTTG
AGTCTCCTCTCTTCTTTATTAGTCTAGCTGGTGGTCTGTTTTAATTTTTTCAAGAAATCAGCTTCTAGAT
TTGTTGATCTTTTTAATGTTTTTTCATGTCTCAGTCTCCTTCAACTTAGCTCTGATTTTGGTTATTTCTC
ATCTTCTGCTATGTATGTCACTGCATGTGAGATGGGTCTCTTGAAGACAGCATACCAATGGGTCTTGATT
CTTTATCCAGCTTGCCATTCTGTGCCTTTTAATTGGGGCATTTAGCTCATTTGCATTCAAGGTTAGTATT
GATATGTATGGATTTGATTCTCTCATGATGATGTTAGCTGGTTATTTTGCAGTTTTTTGTGTGGTTGCTT
TATAGTATCACTGGTCTGTGTACTTTAGTCTGTTTTTGTAGTGGCTGTTAATGCTCTTTGCTTTCCATAT
GTGGTGCTTCCTTCAGAGCTTAGTTTGGCCAGATAATGAAATTCTGGGTTGAAATTTCTTTTCTTTAAGA
ATGTTGAATATGCCTTCAATCTGTTCTGGCTTGTAGGGTTTCTGCCAAGAGGTCTACCATTAGTCTGATA
TTCCCTTTATAGGTGACCTGATCTTTCTCTCTGGCTGCCGTTAACATTTTTTTCCTTAGTTTCAACCTTG
GAGAATTTGATGATTATGTGTCCTGGGAATGATCTTGTGAAGTATCTTACTGGGGTTCTCTGCATTTCCT
GAATTTGAATTTTGGCCTCTCTAGCTAGGTTGGGAAAGTTCTTATGGATGATATCCTGAAATATGTTTTC
CAGATTGGTTCCATTCTCCCCATCTCTTTCAGGGACACCAATGAGTTGTAGATGTGGCATCTTTACATAA
TCCCATATATCTTGGGGGTTTTATTCATTCCTTTTCATTCTTTTTTTCTCTATTCTTGCCTGTCTTATTT
CAGAGAGCCAGTCTTCAAGCTCTGAGATTCTTTCCTCTGCTTGGTCTATTTTGCTATTAATACTTGTGAT
TGCATTATAAAATTCTTGTGGCATGTTTTTCAGCTCTATCAGGTTGGTCATATTCCTTTCTATACTGTCT
ATTTTGTCTGTCAGTGACTGTCTTATTTCAGAAAGCCAGTCTTCAGGCTCTGAGATTTCTTTCCCCTGCT
TGGTCTGTTTTGCTATTATTACTTGTGATTTCATTATGAAATTCTTGTAGTGTGCTTTTCAGCTCTATCA
GGTTGCTTACATTCTTCTCTATACTGCCGATTTTGTCTGTCAGTTCCTGCATTGTTTTATCATGATTCTT
AGCTTCCTTGGACTGGGTTTCAGTATACTCCTGTAGCTCAGTGTTCTTTGTTCCTATCCATGCTCTGAAT
TATATTTCTGTCATTTCAGCCATCTTAGCCCAGTTCTGAACCCTTACTGGAGAAGTGATATGGTTGTTTG
GAGGAAGGAAGATACTCTGGCTTTTTGAGTTCTTGGAGTTCTTGCACTATTTCTTTCTCATCTTTGTGGG
CTTACATTCCTTCAATCTTTGAAGTTGCTGACCCTTGGATGAGGTTTTTTTTTTTCCTTTTACCATATTT
GATGACCTTGAGAGTTTTATTGTAGTATAAGGTGGATTCAGCCAACTGGCTTTGTTTCTGGAAGATTTTC
GGTGGCCATTGCTCAGCCCCCAACTCCTAGACTGTGTGCTCTAACTCTGGGGGGGCTCGTATTGGGCCCC
AACTTTTTTCTCTGGCTCCTTGAAGTTAGGAATCCACTGCATTGGGGGGTCAGAGTGCTCCCAGACCACT
AGTCACTACACCACAATGGGTGGTGGCAGCCAAAGCATTTCATAGTGTGGTGACAGTGGGATTCATCCTT
GTTTACATGTGCCAGCAGCAGTGGTAGCAGCAGTGCAGTGGGGGACACACTTGTCAGCTATGCCAGGGTG
CTGGTGGGTGCCAGGGTGCCTGCCTCCCTGAGAGCATTCACCACAGTGGAGGAGGCAACACAGCTGGGAG
GTGAGCGGGGACCCCTGCTGGCGACTGTGCACAGTGGCACTGGTGGTGGTGTTGGCTTGGGGGCAGGGCG
CTGGCAGTTGCAGGTCTGTGTGCCTTCTCTGTGTACTGCAAACAGGAGTGGTTGCTCCATGCAGGGGAGG
ATCTGTTATTCTCTGTGCCTAGTTTCACTCCTGCGGCAGTGTTGGTGCAAGGAAGGGGCACTGGTGGAGG
CAGGGCTGGCTGGTTTGGTGCCCACCAAGGTTCTGTCTGCAGTGGTGGTCAGTGGGGACATGGGAAATGA
ACTGCACTCCCGCCACAGCAGTGTCAAGGTGGATACATGTACACTTGCCTGCTGGTGGGGCAAGAAAAGC
AAAACCTGCCGGCATAGGCACATGCCAGCAAAGCCATGTGGGGAGTTGCTATGGGCCTAGGGAAGCTGCA
GTATGGGGACTGAATGTACAGGCTGGTGCATAGCCATGGGGGCCACTCTGCTGGAGCTCTCTGCTGGTCA
GGCATAGTCTGCCAGCACAGAAGCTATGATGCAGTCCCCCAGGCCACCCAAGGCTGCCCTGTAAGCATGT
GTGGCCAGGCTGGGGCCCTGGGAGAGGCCAGCAGGCCAAGGGGTGGTCAGATGAGACCGGCCCCATATGA
TGGACAAGACTACCCTGCAGAGTTCGGGTCCAACAGTTGCCTTAGAGCTAAAGTCTCCTATGGGTGCCAG
TTAAGCCTAAGGGGATGGCTATCCCTGGCCATGCCCCACCACAGATTCTCTCACACCAAACCCTCTGGGC
TCCATATCAGCTGGCTTGCTGCCCCTATCACTTCTCTAAGTGGCTCACCCTGTCAACTTGAGTGTTCATG
GTGGGCGAGAGGTTCCAGAGGCCCGTGGCAAGAGTGGGTTGCTCCTTGCTAGTTCAGCTCACTTGTTTTT
CCAGAGCTGTTTGGGCCAGGAATGAGTCCAGGTGCGTAGTCGCCTCATGTATGGTTCCCAGCTTCCTCCC
CCTTCAGCCCAGCTTCTGTGTCTTCCCTCCATCCACTCTTCAGTGACCCCCTCTGAAGATCTGTTAGGAG
CACACCAGTTGTCTCAGTCCCTCATTGGCAGCTTTTCCTCCTGGCTGTGTCTAGTCAGCCATCTTGCCCT
CCCCCTGGTTTTTTTCTCCCCCGTTCTTTGTCTTCAATTTTTAAAAACCTTGGTCAAAGAACTCTTTAGG
CTAAGTTTGAGAACTCCGTAGTCAGCTTCCATGCTGTTAGCAGATCTTATGAAATCATTTAAAGTTTTTA
TAATTCTGCAGGTGGGTCTTGTCTAACATGTGATAGAGGGCCAAAAGGCCACACTACTGAAAGCTTAAGG
AGGGGTGTGTGTGTGTGTGCGTGTGTGTGTGTGTGTGTGTGTGTGGTGTTTTGAGGGTTGCCTTTTCTCT
TGGTTGTGGTATGTTAGGAGTAATGGCTGTTACATACTGATGTGCTTGATGTGCTTTGTCTTTACGGGTT
ATAGCAGTGTAGACATGAGTCTCCTTTTGGGATAATCTGTTTCCCTTTTTTCTTCTGTCTTTGCTAAAAG
CTATTGCTCATTTCTGCTGAGCATTGCAGTTACCCCTTCTACCTACCATACTTTCTAATTTTGGATCTTC
TGGTGGATTGTGGGCTAGGAAATACGAATAGTTAAGACTCTTCAACTTGTTTTAATCAAACAGATGAGTC
CTCATGATCTTTATCAAAGCAAAGTAGGTGGGCTTAGGGTCAGCCTCTTTTCTTTTCTTTTTTAAAATGT
TCTTTTCTTGCTAATAAAAGCAGTAGCCATTGGAGAAAATTTAGAAAAGACCAACTATGAAGCAAAACAT
AATCATAATACCTTGAATCAGAGTTAATCTGTATTAACATTTGGTGTATTTCCTTTAAGCTATGTAAGTA
GACTTCAGTAAAAATCCTTCACAAAATTGGGATTTTAGTATAGTATACTTTTTATATCGATATGTCTTTT
AAATGGTAAGCATTTTTCATGTTAACTATTTTAATACAGCTTAATGTTTGCATATTTTATTATGTACATT
CATTCATTATATTCATTCACAGTTTATTTATAAACATACAGATTTTTTCCAATTTGTCCAGTTTTTATAA
AAGAACAAATCCCCATTGACTTTTTTTTTTTTTTTTTTTGAGACTGAGTCTCACTCTGTTGCCCAGGCTG
GAGTGCAGTGGTGCCATCTCAGCTCAGTGCAACCTCCACCTCCTGGGTTCAAGCGATTCTTGTGCCACAG
CCTCCTGAGTGGCTGGGATTACAGGCACCTGCCACCAGGTCTAACTAATTTTTGTATTTTTGGTAGAGAT
GAGGTTTTGTCGTGTTGGCCAGGCTGGTCTCAAACTCCTGAACTCAAGTGATCCGCCTGCCTTGGCCTCC
CAAAGTGCTGTGACTACAGGTGTGAGCCACCGCACCCGGCCCCAACAAATTCTTAACATCTACATTTATT
TTCTTCTTTTCCTTTTTTGTAGAGAAAGGGTCTCACCATGTTGCCCAGGCTGGTCTTAAACTCCTGGGCT
CAAGCCATCCTCCTGCCTCTGCCTCCTAAAGTGCTGAGATTACAGGTGTGAGCCACTCTACCTGGCTCTA
CATTTATTTTTTTTACATGGATTTCTAAAAGTAGTGTTTACCAAGTCAAGGTTAAGAAATTTTTTTTTTT
TTTTTTTTTTTTTAGACGGGGTCTTGCTCTGTCGCCCAGGCTGGAGTGCAGTGGCACGATCTGGGCTCAC
TGCAAGCTCCGTCCTCTGGGTTCACACCATTCTCCTGCCTTGGCCTCCCGAGTAGCTGGGACTACAGGCA
CCCGCCACCACACCTGGCTAATTTTTTGTATTTTTAGTAGAGATGGGGTTTCACCGTGTGAGCCAGGATG
GTCTTGAGCTCCTGACCTCGTGATCCGCCTGCCTCAGCCTCCCAAAGTGCTGGGATTACAGGCATGAGCC
ACCGCGCCCAACAAGGTTAAGAATATTTATAAGTGTTTTAATATCGACTCCCACATTGCTTTCTGGAAAA
GTCATGTGCTTTGTATTTCAGCACTTTATGGGTCTTTGATTCCAGATAACTTACCCGCACAAGTTTCGTT
TTATAAGATCTTTGCTAATTTGATAGATATAAAATGCCATTGGATGTTTTCCTCTGATTGCTAGTGAAAT
TAAGCCTTTTTTCATGTTTATTGGACATTTATATTTTTGTGGGAATCATTCTTGTCCTTGAGTTATTTTT
CTGTTGAGAGCTTAATACTTTTTACAAATTTGTAAGTCTTTTGTGTACTAAAAGTTAAAAGCCCTTTGAT
TTTTTTGTAAATATTTTTCTCAATTTATTTACCTTTTAATTTTGTTAATTGGATTTTTTAAAAATAGAAG
TTTTACATGGTTAAATTTATCATTTTAAAATTACTTTTTCATTAGTTTTAAGCTCAGAAAATTTCCTTTC
ATCCAGAAATTCAGTTACATCATTCACTTACATCTTTTTTTAAAACTAGACTTTTCCATTTTATTGGCTG
TATTTACCCATTCTTCCAGTTTGAACTTTAGAATGACTTTTTCAAGTTTCTTACAAAAATAAATTCCACT
ATAATTTTTTATTGGAATTGCATTTAGTCCTCATTCTTATAAGTAGCTTATTTCTAGTACTTTAAAAAAA
TTTACTGTTGTAGATTGTGATTGTTTACTCCCTTGTGTTCCCTGTTTTCCCTCTCCCTCCCTACTTACCA
GTAGATGAAATTCTAGCTGGTTATTAAAAGAAATACTGCTGGATACTTGTGGAGGTGAAGGCTTGGCAGA
AAATAGGTGTCACTGGTATCAAATCATTGAAAAGTGGGTTAGGATAACTTCCTTACCATGGTTGGTGGAA
CTCGTTTCTCTGGAGTTGGGTGAATGGTAAAGCAGGCACAAACCTCCTTTTGTTTTCCTGTCTGACGTTA
CTATTCTGTACTTTATTGTAGAGAAATTATTCCTAAGGTCTTCTAGATTTTTTATGAAGTATTGTTGGTT
TCCAGGACTCTCAACACTTTATATTTCTTATTTTCATGAGTTTTTTTGATGAAAAGGGTTGAGTAGGATA
ATCAGACAGAGAGACACAGTTTCGGCCGGGCGCGATGGCTCACGCCTGTAATCCCAGCACCTTGGGAGGC
TGAGACAGGTGGATCACGAGGTCAGGAGTTCAAGACCAGCCTGGCCAAGATGGGGAAACCCTGTCTCTAC
TAAAAATATAAAAATTCGCCAGGTGTGGTGGCATGCGCCTGTAATCCCAGCTACTTGGGAGACCGAGGCA
GACAATTGTTTAAACTTGGGAGGCAGAGGTTGCAGTGAGCCAAGATTGCACCACTGCACTCCAGCCTGGG
CGAGAGTGAGACTCCCATCTCAAAAAGAAAAAAAGAAAAAGAGAGAGAGTCTTCATCTAAACCTAGAAAT
TCTAAAAACTAGCTGTTTGTTCACTATTTTTTGGCAGTGTAAAGAATTCTTAAATAACTAGGTATTTGAC
TTCCTAAGAAGTGGGGTATCACCATATATATGCATTTGAGAACTTGAATTTACAAATAAATACAGAGATT
ATTTTCTATTTTTATATCTCAGGGATGTTATTTCAATGCTAATAGTGAAAAATACTACCACCTTTCTGCT
TTTAGGTTGGAATGTTGCATCAGCAGGTAGAAGAACATGAAAAAATCAAGCAAGAGATGACCATGGAATA
TAAGCAGGAGTTGAAGAAACTACATGAAGAAGTGAGATTTTAGTTTTGTTAAATGTTGTGTGTGTAGTTC
TCTCTCTTTCACCCTTGAGAACTTTGTTAATGAAAGCCAGTTTCTACTTTGTAAAGTGGGAAATACATAC
TCCATCAAAGATACAGGAGTGGGAATCCATTTCTCTGTTCTTTAATCCAGGTAAAGAGGTTTGCCTAACT
GTAGGTTTGATACTACCTAGAAGAGATGCTAAAATATGTTTCTTGGTTGAGTACCTGAAAGAAATAGAGC
TGTTTTTATGTAATACTGTGAAATACTTGATACCAAAAATTACATTTAATATTTTAATTTTTATCAGTTA
CTAGATTATAAATAATGGAGGATCCAAAGATTTAAGAAACATTTTGGGATAAATAAGACAAGTAAGAAAA
TTCTGTCCATTAACTCAATTTTATTCTTCAGATTTGGAAAAAACTGCTTTTATTCTGATTTAATTGCAAA
GTACTCTTCTCCTGCATCCTGAATATCAGTGCCCTGCAAGAGCATGGCTTGAGAATCACAGACCTAGAGG
AAAGAAAAAGAGTTTTTATTTGCTGCTTTATTGTGGGGCTGGGTGGTAGGGTCTTTAAGTGCAGGGCTAT
TGTAATACAAAACTTTTGTTTAAAATTTTTTTTAAAATTTTTTCTTTGAATATATTTCTTTAGTAGTGTT
TGTTTTACATATTTAATTTTTAGTTTCCTTTTGTGTTTTAGTTTGGAAATCTAACATGTTCTCAGTAGTA
CTGGTTAGCACTGCTCTTGTTTTCATAATACCAATTTGTCTCACCACTACTTCTTACAAATTCTTAAACT
TTAAATATAGAAATAACTGTTTCTACAGTTATGCATACTGAAGAGAAGCTATGAAAAGCTTCAGAAAAAG
CAAATGAGGGAATTCAGAGGAAATACCAAAAATCACAGGGAAGATCGGTCTGAAATTGAGAGGTTAACTG
CAAAAATAGAGGTATGTTCATAGTAATAATTTGTTCATAGTGATTGTCAGCCTTCACGTAGGAAAATGCG
GTTTCTAATGGCACTATTTTCTGGAACCAATTTTCTACTATCTTAAGGACTGCCACTAAGCTATTTCAGA
GGTGAGAGGGTGATCCACCATACATCCTATCATCATCTAGTTCCCTGGGTTAACGGGGTTACTGGCCATC
AGTAATTCTCAACAGGGCAGTACCACCCTTAGGGAGCATTTGCAAATGTACGGAGACATATTTTTTTGGT
TGTCAAAATGACTGAGCTTAATAAAACTGGCATTTAGGGGACAGGGACCAGGGATACTAAACATGCCTAA
GAGTGTTCCACACAGGAAGGATTATTGTACCTCAAATGCCAGTAACTCTCCAATTGGGAAACATGACCAC
TAGTTATAGGCAGTCCTCTATGGACATTGTTCTTGTCTTATATATCATTGTATCTCAAGCACCTAATAAA
GTGGGGGCTTAGTAAATGTGGAAAGTACATGGCACTTTTCGTACAAAGTAATCTCTGTATCAGATTATTC
TCAATTCCCATTTAGATGTAGTAAGGCATTCACTTCCTTCTCTGCTGACTTTGTTTTTATTAAGACTGAA
TAACTCCATGATTATTGTATATATTGAGAACTTAAGGGGAGCTGGTGAGCTAATAGTACCAAGAGTAGAT
GAAGGAAGAGCACAAGGCTGTCATTCTCTTCGTCTGTCCTTGCCATTAGTTCTTCACTGGACATAAGAAT
TCCCAGGGTGCTTTTAGGCTCTCAGAAATGCTGATCCAGCAGTATGGCATAGGACCTAGGGATCTGCATT
GTATCCAGCTCCCCTAGCTTATTCTGATTTGAGAAACATTGTTCAACATCTTTGTGTGATCACTTACAAG
TTTGTACTCTGGGTGACCTGTCACCCACAAATGCCAGTATATTATTATTACTTAAAATTATAATTTCTGT
GTTTAATATTCCTTTATATCTCAATATTTTTTTACATTTGAAGAGCTTAGAATCAGAAGACTGCAGCTAC
TGCTTCCCTTCCACCCTTTTCGGCAATACCTTTTCCCCCCAAACCCTATATCCTTCTTGCTTCTCATTGC
CACTTCCTGACTATTCTCTTTTCTTTCTACCTCAAAAACCCCATCTTTGATTTTCATGTTACCAGCCTAT
ACTGCCAGCCAATGAACATAACAACCAAAATAGTCATCGTCTGACCTCTGGGTTATTTCCTTGGCTCATT
GATTTCAGCACCCAGCTCACTGTCAATTCTCTCTGACATTACTCTTATCATAACTTTGAGGATCTTAGGA
TCTGGAGAGATGATGCTTATAATACTCTGACCTTACAGTTTCTTGGCTTTCTTTCCTCCATAGATAGTTT
GATTATCTTTGTGTCTCATCTACTTATTCCCACTGCCATTCCTTAGCTCTTGTCATTACCAATATATGCC
ACCCCTCTAGAATCTCATTTTCAAGCATTCAATTATCAGACTACCAATCCCTATCTTTTCAGCTAACTCT
CTCTAGTCCTTTGACTCTCAGTAATCATTAACCATCCCTTAAAATGATTAAATTCTGCCACCTTTTTACT
GCTCCATTATCTACTTGGTGTTCTCACTTATCGGTCCTTAGCCAGCTTTTAAATTCTGTCATTACATTTT
TTACTTTCCCCTTCATCTCCTGTGCTCCTCTCTTGCTTCCTCATATTTGCTTGGCAGAATCACAGTTCTG
CTTAAGTCCACCTTTCTGCCCACTCTCTACCTGTCATGTATCGCTGAACGTGGCTAGAGAAAAACCTAAT
CATACTGTCTGTTCTCACTTTAAATTCATGGTCACTAACCTCAAATGGGTCCTAATGCTACCTGGAATCA
TACTGTATTTCCTCTCATCTCCTACTTTCCACAATGAGTATTTCAGACTTCTGCTTTTCTCCTCAAACTC
TCAACACCTTTCCCCACATCCTCACCTTTGGGTTATATCTTCCTTTCTGTTTATAGACTTCCAACTCTTG
TGTCCACCCACCTATCTGCATCTGTGCCCATGTACCCTGCCTTCCTTCCTGGAGGAACTACGCATGCCCT
CCCCCTTTTCTACCCAAGGCATTACCTTAGCAGTTCACCTTCTCTTTTCTGCACCCTTAATTTTTTTTCT
CTACTGGATATTTTTTACCAGCATCCAAATTTCCCTCCTCTTAAATATCCTCTCTTGACCCGTCTCTTCC
TCCTCTGCCCCATTTTATTTCTTCCATTTTATAACTAAAAAATTACTTCAGTCATCTATACTTGTCTCCA
GTTCCTCTTGTCTCATTGTTCTTGTACCCACTCAATAAGGCTTTTGTCCCCTCACACTGCAGAAGCTGTT
CACATAGAGGTCACCAGTGACTTCCACATTCCCAAAGCCTGTGGGCTGGTCCTTATCTTCCTCATCCTAC
CTACAGCTTTGTAGGAGAGCTGGTCTCTCCCAACTCCTTAACCACTTTCTTGACTTGGCTTGAGAAAAAC
CACATTTCTCTCATTTTTTCCTTGTTCATCACTCCTCAGTCTCCATTATTCCTTAACCACCTAGGGTCAT
GTCATTGGACCTTTCCTTTCCTTTTTTTTTTTCCTATCTACGCTCACTCCCCTATTGATCCCATTGAATT
TCATAGCTTTAACACTGTGTATATGCTGACAATTCCTAAATTTATATATCTAGCCAAGACCTCGCCTCTT
CATGACAGAATAACTTTCAACTGCCTGTTTCCTATCTCCACTATGATGTCTAAAATGTGTCAAACTTAAC
ATGTCCAAGCTAAAGGTCCTGTCTTTCTCTTCCTCATCTCCCCAACACTGATTCATGTTTTAGTTGATAA
CAACTCAATTTGCTCAACCTAAAACCTCGGAGTCATCCTAGACTTCTCTTTTCTCAGCATGATAGCCAGA
ATGATTCTGCTGTAATGTAAGTCAAATTATATCACTTCCTATACAAAAACCTTCCAGCAGCTTCTCTTAA
AAATCTGTTATCCTAATGGCCTACGTGGTCTCATTCCCAGTTATCTCTCTTACCTTGTCTTCTCCTACTT
TCCCTTTCATTCACTTTATTCCAGCCACACCAATTTCCTTTCTCACCCTTAAACACACCAGGATGCTCTT
ATTTCAAGGCTATTCTTTCCACCCAGAATATTGTAACTTCACCCTCATCTCCCTAGGTGTTTACTTAAAT
GTTTTTCACTCAGTCTGGCCATCCCTTTTAAAAATTACAACCCCAACTGTGAAGACTTCATGCTCTGTTT
TCTCCAAAACGTGTATTACCATCTGCTATACCATACAAGAAATGTGTCATCTGTTGCCCCCAGTTGAGTA
GGACTAGGGTTTTTGTTTGTTTTGTTAAGTATTGTATCCCTGACTCCTAAAGCAGTACCTGGCATATATA
GTAGAATGATTGAATGAATGAATGAATATCAGAAATTTGGTTAACAACACTTATCTTAAGGAGCCTGAGA
CTTCTGGAGTTGAGGGTCCTGGAAGACAGTGATAGAAAGATTGAGTTTCCTTTCCCTTTGATTTGCCCCA
TTTTCATAAATATTGAAATCACTTTCTTGATCATGCAATTTGATGTTACTATTGATCCATCATTAATTTT
GAACTGTTTGGCTTTGGGTTACAAATATTTGTTAAAATTTGGTACAGATTTTCTTTGTTATTCAAACTTT
TGCAGTTCCTCTGTTGAGATAATACAAATTTGGATATCAAAGGATATTGTGTTATCTGAATCTACTTGGT
TCCTTAGTTTTAGATACTGGGAAGTAAGATTTGGGTTACCAAGGGACTTATCTGAAAATTTAATCTGTTC
AATTCCTGGGTCTGGATATCAAGAATTTGGATAGCGAGGGATATCTGTAATCATACTTTAAAATTTTAGT
TATCTTAGCATCAGTTTTCTGAAAATTCACTAGAAAATTATAACAACCCTTAGGTGGCAGCAAATCAGTG
AAAAACATCTGAATCCAGTGCTTAACATCTGCGTGTCATACTCCTCACCATCAGGGTGTTAAATTTAGAA
AGACAGATATCCAGAGGCACAGTATGTGAGGAAGTTAGTATTACTCTCCAGCTCTGTTAGCTGAGTGAGT
AATGGGGCTCCCGCTGTGGAGAAAGGTGCAAGCGTACCCAGGGACATGGGGAGAAATTAAAGTTAAAGGG
AGTCTGGGAAGTAGTGACAGTGAGGAGAAGCACTCAGAAATTAAGTACTCTAATTGCCTCCTCAGGAATT
CCGTCAGAAATCGCTGGACTGGGAGAAGCAACGCTTGATTTATCAGCAACAGGTATCTTCACTGGAGGCA
CAAAGGAAGGCTCTGGCTGAACAATCAGAGATAATTCAGGTAGGCCTAAGACTTTTTAAAATAATGAGAA
GCAGATAGGTTTTGATCAAGTTTGAACTACCTTAATGCAGTGTCCTAGTACCATTTAGCCTCTTTCAGCT
TCTCTCCACGAACCCTGCTCTCTTGTTTAGGAAGCCTCTCCTCAGCATCTAGACTCAGCTCCTTCTTCCC
GCCTGCCCTCACACATGCGTGCATTCACTCTCCTGTCCAGGCTCATGTCCTTGTGCCCGCTTGCTGCTTT
GCCTCCTTGCTTGTTTCTGACTTGCTCACCCACTCTCTGTCATTGTCTTGCTTACTCAGGCTCACTCTAT
GCTTGCATCCATGTTCACTCTCCTCATCTTGGTCCCTAGCTTTGTCGCCCTCTCTTCCTCCCCTCTCTTT
CTTCCCTACCACAGTCCCATTCCTCCCTCTGTCTCCTTTTCCATGTCTTTCTGTCTCTTGTTGGGTCTCT
CAGCTCTACTATAGTCTGTGCCTTGCTCCATCCCTATGGTCACTGGGGTATCTTGGCCGAAGTGTCTCAG
CTAGGGAGCTTCAGTTAGTCTGCCTATGCGGACAGATACGGAGAGAGTCACATGGCAATGGTGTGCACAC
CAACTAAAAGTGGTGTCTTTGTGGTAACAAAAACACTTATTTTAGAACATTGATAGAAACTTTTCTTGTA
ATGGCTTAAGCAAAACCAAAAAACAGACACCTACTTTAGGAATATGTCTTGTAAGAACATATTCAGCTCA
TTCCTTTTTCAGTTAGAATCAAGAACATAAAGGAAACTAAAAAAATTTGTGATCATCTGTTATGTGCTAG
ACATTGACCCAGTGCTTCTTGTATGTGTTCTCTGTTTAATATTATGATAATCTGTCAAGGTTGGTAGTTA
TCTTAGTTTTATAGAATGAGGGAACAGCCTGGGTAAGTTGAAATAGCTAGAAGGGAGGGTTTTTTTTTTT
TTTTTTTTTGGCTTTTTGGGTTTTTAAAAATAATCTGCATTTATTTGCTATTTTAAGTCTCAATAATGCA
TTAACTTTTTGTTCGTCTCCCTGAAATATCAGCAGTCTGATAAACTACCGACAGTGGCTGTGGCTCTGCT
GTGAGTGACTTCATTCAAAATGGTAGGAAAAGTCCACAACAATTGCAAGGAAGAAATTTATTTTCTAGGC
CACAGCAAGCTGTGCAGCCAGACAGACCAGTGAAATCCTGAGATGTCTGGACCAGTTTTTTTGACTTGAA
TGCCAAAGCACCATAGAGACCTAATAAGAAATTGTGTGTGTGTGTATATATATATATATATATATGTATA
TATATATTTTTTTAACAACAAAAAATTTTTTTTGGTTTTCTTGAGATGAGGTCTTGCTGCATTGCTCAGG
CTGATCTTGAATTCCTGGGCTCCAGCAATCCTTCCACCTTGGCCTCCTAAAGTGCTGGGATTACAGGTGT
GAGCCACCACACTTGACCAAGATATTGTTATATTCTAGTTGTAGCCTTCAGATTGACAAAAAAGCAAATT
GAGTCTTAATTTTTACAGTTCTCAATTATTTATCCATGGAGTATTATTGATTATAGATTCTCTCTTCAAA
TTACTGTTATCCTATTTCATATTACTGTTGTGATTATTGACTCTATGTAATTAAAACTTCATTATTAGTA
AAAATGGGATTTTTACTTACTGTAATTCAGCTAGGGCTAGGGTAAGAAGCCTTTCAACAGCCAACATTTG
TTTTTCCTACTAAGGGTGTTTGAATTAATTCTTCTGATAGTTTTGTTTAACCTAAAACTGTCTGATTGTT
TAATAAAAATGTTACATATAAATGTATTTATCCTTCTCCCTTAGCAAACTAAATAAAATTAAATCTTTCA
GTGATTCAGTATATACCAGTGCTGTATGATCATCATTTTTTTAAACATCTAAATAAACTTATCTATATCC
ATTAAGTAGCATTAATAGTTGTCATCATCTTGTCAATCTTTATTTTAATTATGGTAAAATACACATAAAA
TGTTCCACCTTAACCATTTTTAAATGTATAGTATAGTAATGTTAACTATATGTACACTGTTGTGCAACAG
ATCTCTAGAACTTTTTTATCTTGTCAAACTGAAACTCTATACCATTCCCTACTTCTTATTTTTGGGTGGA
ATATTACAGAGCAAATCATAGATATCATCTTATTTCCCAACAGGTAAGGACTTAAAAAATAACAATATAT
CAGTATCACATTAATAGAATAATAACTTCTTAATATAATTTAATATCCAGTCTGTTCACATTTCTCCAGT
TGTCCAGAAAACTATCTTTTTACAATTGAATGTTTGAGTCAGAATCCAGTCAAGGTTGATGTATTGCATT
TGGTTGAAAGGTGGGTCACCATTGAACAATGACAGTAGAAAACCTGGGAGGCTGAGTAAGTGGATATTAA
CCTACCCTCCATTAAAAAAAAATCTCTGTATAATTTTTTTCTTATATGTTTTTAACATCTAAAAGTATAA
TTCGGAAACAACTTCTCCTTCTTTGACATATGTTTGCTTGAATTCATTTCTTTTAATCAGCTATTAAAAT
TACATAATACATGCTTATTTTAGAAAAATGAGAAAATGCGGATAAAATGAGAAATAAATAAATCACCCAT
AATTCTACCGTTAAGAGATTTACGATGAATAAAATTTTGGTGTAAATTTCTTCTGACTTTGTGTATGCTT
ACATACAGTGTGCAGTACATTCATGTACTTTTGATAGGAAAAACCTATATGCACTGTGAATGGGAAAGGA
GGGGTAGTTGCAAACTTTAAAAAGTCCTGAGAGTAGCAAAGTGTTGATAATTTTTGAAGCTGTGTGATAG
GGGTTCATCATCTTACTCTCTCTATATTTGTGTATGCTTGAAATTTTCCATGAAACAATAACTTTAAAAT
ATATAGAATGCTTACTAAGTGACAGGTACTATGCTAAGCACGTCACAGCTTTAAAATTTTTCCTTTTTAA
ATATGACATGTCAACAGATTTGTAGTCATAATGATTGTGTAGAATTCCCCCCTATCGTTAAATGTATAAT
GACATTTGTAACCAGTCCCTTATTGTAGAACATTTAGTTTTTTACTTTTCACTTTTGTAAATAGTGCTTA
GATTAACATCCTTATATAAACATTTAGGAGTATTTATTTTCTTAGGAAATGGCCCTAGAATTGGATTTAC
TGTATCAGAGTACCTTCAAATATTTTAATATATATTGCCAAAGTTTTCTCCAATGATGGATTGATAGACT
TTCTCTAGCAGTATGTGAGAATATCCATTTCCTCATACTTTCCCCACATTGAGCGCTATTATTTCTTTTG
ACTTTCCCCACTTTTAGGCTTGTCTTTGCCAATCTTATAGGTGAAAAAAAATGATACCATTGTTTTAATA
TACATTGTTTTAATATGAGTGAGGTTGAGGACTTTTTTCATCTGTTTATGGCCATCTTCATTTTTCATAA
ATTATTTGTGTCCTTTATTCACCTTTGTTTTAGGATTTTTTTTTTTTTTTTTTAAATCTTAATTTATTTT
CTTTTTTGAGATGGAGCCTCGGTTGCCCAGCCTGGAGTACAGTGGCCTGATCTCGGCTGACTGCAACCTC
TGCCTCCCTTCAAGCAGTTCTCCTGCCTCAGCCTCCTGAGTAGCTGGGATTACAGGTGCTCACCACCACG
CTCAGCTAATTTTTGTATTTTCAGTAGAGACAGGGTTTCACCATGTTGGCCATGTTGGTCTTGAACTCCA
GACCTCAAGTGATCTGCCCACCTTGGCCTCCCAAAGTGCTAGGATTACAGGCGTGAACCATCGCACCTGG
CCGGATGTTTATATTTTACTATTCTGAAAGAGCTTTCCATACACTTAAGTATGAAATATAAGCCCTTTGC
TATTATGTTACAGTTGCGTATGTGTACTCAATTTACCTTAATTTTTGCCCAGTATTTTTTATGGAAGTAC
AATTGTTTTATAAACAGTTAATTAGAATTGCTTAATTCTATCAATTTTTTGTTCAGCGTTTCTATCTTTG
ATATCATACTATAAAGTCTTACCTAAGTCAAATTTCTTCTAATGCTTTATCATTTCTTTTTAATACATTT
TATCTAAAAATTACTTTAGTGTAAGGTATTTGATAGGAATCTAAATTTGTTTTTTAAATGGTTATATAAT
TTACTGACTATTACATTTTCAGGATTGGACAAAATGACTCTAAAATTCATATGGAATGATAAATGAACGA
TACTAGCCAACTTTTTTTTTTTTTTTTTTTTTTGAGACGGATTTTCGCTCTTGTTGCCCAGGCTGGAGTG
CAGTGGCACAATCTCGGCTCGCTGCAACCTCCGCCTCCCAGATCAAACGATCCTCCTCCTCAGCATCCCA
AGTAGCTAGGATTACAGGCGTGCACCACCATGCCCAACTGATTTTGTGTTTTTAGTAGAGATGGGGTTTT
ACTATCCTGGTCAGGCTTTTCTCGAACTCCTGACCTCAAGTGATCCACCCGCCTCAGCCTCCCAGAGTGC
TGGGATTACAGGTGTGAGCCACTGCGCCTGGCCACCAGCATTTTTTAAAAAGGAGTAATGGGGGGATGTT
TGTTCTATCAAATATTAAAATGTATTTTATTAAAAAGTACAGTTGTTAAACTGTAATGCTAGATGTAACA
AAAGAGTCGTGGTTATATAGTATAGGAAAAGGATCAGTACAGTCTAAAACTAAGGTCATTTCTGTTTAGA
AGGCTACTTTTTCTGTTGCCTACATAATGCAAAGGAAATGTTCAAGGAAATCTTAGAGAACAGTAAGAAC
ATTTGAAAAAAATTAAAATATTAAGCCAACATAAAATGTAAAATATTTAACTGATAAACAGTATGTGAAA
AAAGTAGAGGTTGCTCTATGTTTTCCTTTTGAAATACTTAGATGAGCTGTAAAATATTCTTCAAGTTAAT
TTTAAATCCACTGTTAATTATATGTATATAAATATATTTATACGTATATATGTTGTATTTTTAAAAGCAT
CCTTTTCCTGTCACAACTTTTGAAGAAAAAAATGACCCTAACTCAAATGTTGATCTGTTGTCTTGGTAGA
ATTATCAGAGTTAATTGAATTGGCTTAGACCTACTGTTTAAAAGTTCGTAGTAGCATAGACACAACAGTG
ATAAGAGAATCTTACTAGGAGCTGTGTTGTTAATAGGCTCTTTAGGCTGCTCTCTAGTGGAAATGCTGTG
AGGCTGTGGCCTGCACTGGCGCATTTGGAGTTGACAGTGATGGGGAGCTTGGTAAGGGATTGATGCTTAC
AATGGCCGTACCTTCATACTGGGATTCTTTGGAAAATAAAAAGCCTTGTTGCTTCATATCACATTGTTCA
TAGTAATCTCACTCTCTGGGAGAGTCATTTTCTTGAAGCACCAGGAAGTAGCTATCACAAATCCAGAAGA
GGATTTGAATTCAACCCCCACTCCCCAATTTCTTCTCCACTCTCTTTCACACTTTAGCTGGCCACACTGG
CCTTTTGTTTCAGTTTCAGTCTCCCTGATGTAAACTGCCTTCTTGATTGCTTCTTTACTTTTAACCACCT
CTGCTGTGATTTGTATTGTTGAAATAACTTCTACCCACTCCAATAGAATGCTAAATGTTAGATTAATAAG
AGGAATGCCTGTTTCTGGCTCAAAGAATTAGTGGTTAGTGCAAAAAAAAAAAAAGTTTAAAAATATGCCA
GCCTTAAAATACCTCTAAAGCCAGATAATTATCAGAAATAATCATTATTTTAGAAGTTTTTAAATAATTA
TTTCTGACTTTCTTAGTATTGATGAAGTGTTCTTGAGATTAGTGTAGTGCTAATAGATATTGGTGAGTCA
CATGTAATTTAAAATTTTCTACTAGTCACATTAAAAAGTAAAAAGGTGAAATTAATTTTAATAATATATT
CAACTCAATATGTCAGAAATATTACAACATTTACTTTACATTTTTTTAATGAGTCTTTGAAATCTTCAAC
TGAATTTGTATTTTACGGTTAAGAGCACATCTCAATTCAGATTAGCCACAGTTCAGTTGTGCAATATCCG
CGTGTGGGTGACTGCAATGGTATTGCAGCCCTAGTGCATTCTAGGCAATGAAAATTTAGAATTTGTACTT
GAGTTGATTAATGTACTTTTCACTATGCTTAAAAAACACTGTATTTTTGTGTGACCAAAATAGTAAATCA
GTGAAAGAAGGTTATGTTCTCCATTCTTCAAAACTGAGAAGTTTATTAGTATTTAGCAAATCTAGACTCA
CTGGTTAAAGTATGCCTTGGTTTAACTATTAGTTTATTATATCAATACTGAGTTTAAGATCACAGATTTG
AGTTGATAGTGTTAGGTAGCTATTACAGTATTAAAAAGATAGTGTTTTGCAATTATGTATTATCTGAGTG
CCCATTTTGGCAGTGGGTTTCTAGTTGTGTTTTGCTGAGACCCAGGGCTGCTGTACAGATTAAGGGTAGG
ACTAAGGGACATTTCAGCAAGCTGGGCTCTAACCCTTTTCTCCTTCTCACCCTCACCCAATATCTGCATT
TATTTATTCTGCACAGGAAACCTTTCCTCTTTGAAAGAAAAGTTTATATTATATTTAAAAGTTTGGAGAC
AGTTCCATGCTTAGCAGTTGTTCTAGTGAATCTAAACCTAAATGTTGATGTTCTTCCTTTATACCATTGT
AGGGATCTGGTTAAAAAAAAAAAAAGGCTCTTTGCACTGAAATCTTGATCGGTAATTTATACAAACAGTA
TTTGTCTATCTCTTCACCTCTCTCCTACCCTCTCTCTGGCTTATGGGAAGTTTTTAGCATCTACTTTATT
TGACTACAGTATATAGTACAGGATTTGGACCTGTACTGTAAATAATAAAATCTTTAAATCTGCAATAGAT
AGATTTCCCAGAGATATTTCTACTTGTATTTCTAAAGTAAAATTGTGTGATTTTACCTTTAGAAATATTT
CGAGTAAGGGAAGTTTACCTCTACAGATTATTTATATTCATCTTCAATGTGTAGATGAAGTACCAGCCAG
GATGCTGGATTAAAACTTGTAAAAACTAAAAAGTTTGGAGCTAAAACCCAATACTGCTGGGATGCATAGC
CTAAAACATAGTAGTCAGTAAACGTATCATTCAGAGAAACCAGTTTGAGATGCATTATATTTGTTTACCT
CTTTTAACTCAGGAAGTGTCCAAAAACTGATGACAGAACCAATCTCTCCTCCCTTATAAGCACCTTTTCC
TCCAATAAACAAAAGACCACAATTTCCCCATTTCTCTGAATTTAAAGAGCATTTTATGGTCACGATGACT
AATCTAAATATGAATGTTTATAATGTTAAAACAGAATAATTACAACATGCTCTAGGTGGGGGGGGGAATT
TAAACTACAAATTTAGTGATTTTGAAATTCTAATTTCTAAACTTCCATCAGAAATATAATAATGCATTAT
AGTTCCTATTTCCTATGTTTTAAATAGGAAATAATTCCAGCCAGGTTGCGGTTGAATTTCTGTGTGAGTA
GGTAGAATACTGTAGCATCTCTTCTCCTGAGGAATCCCTTTACTTTATCCATTCTTTTCTATTTATTTAT
TTATTTGAGACAGGTTCTTACTTTGTCACGCAGGCCGGAGTGCAGTGGTGCGATCTTGGCTCACTGCAAC
TTCCGCTTCCTGGGTTCATGTGATCCTCCCATGCCACTGTGCTCGGGTAATTTTTGTATTTTATTTGTTT
GTTTGTTTGTTTTGAGACAGAGTCTCATTCTGTCACTAGGCTGCAGTGCAGTGGCGCAATCTCGGCTCAC
TGCAACCTCCAACTCCCTGGTTCAAGTGATTCTCCTGCCTCAGCCTCCCGATCCCGAGTAGCTGGGATTA
CAGGCACGCGCCACCACGCCCAGCTAATTTTTGTATTTTTAGTAGAGACGGGTTTCACCATGTTGGCAAG
GATGGTCTCAATCTCCTGACCTCATGATCCGCCCACCTCAGCCTCTCAAAGTGCGGGATTACAGGCGTGA
TCCACTGCACCCGGCCAATTTTTGTATTTTTTATAAAGACAGGGTTTCGCCATGTTTCCAGGCTGATCTC
GAACTCCTGAGCTCAAGCGATCTGCCTGCCTCAGCCTCCCATAGTGTTAGGATTACAGGCGTGAGCCACC
ACTCCCAGCCTCTCAATTCTTTCTTACCCCTTCGCAATATATATATATATATTTTAGTGAGCCAATGCTT
TATGTTTTTATGGAAATAATAGTTTCATTTTAGTCTTATTCATTGATTATTTTATCATTTCCCTTTTACC
TATTGATTGATAGTCTGTGTTTCTAGGCTCAGCTTGTCAATCGGAAACAGAAATTAGAGTCTGTGGAACT
TTCTAGCCAATCAGAAATTCAACACTTAAGCAGTAAACTGGAGCGGGCTAATGACACTATCTGTGCCAAT
GAGTTGGAAATAGAGCGCCTCACCATGAGGGTCAATGACTTGGTTGGAACCAGTATGACTGTCCTACAGG
AGCAGCAGCAAAAAGAAGAAAAATTGAGGGAATCTGAAAAACTATTAGAGGTATGTTTTAAAATCACTAT
AATTGGGGGAAGTGTGTGTATGAGTATTTTAAGAGAGTGTTATTAAGCTAGAACTAGGTTCTACAATCCA
TACTTTCTTCCCTACTTTATAAATGATGGATCAGATATTGATAGTTTTACTAATATTGTGACTTTATTAC
AATATTTACAATATTGTAATATTGTTACGTAACTTATAGTCTGACTTCCTGGCTTCTCGTGAAATAGAAA
AATTGTAATAATAGCTGGCTTAGAAATTTCTGAGTATTTTGCAGGTTCTGCAGGAATTAAAAAGAAGGAA
AATTATAATCATATTTGACTTTTTTGCAGGCTCTGCAGGAAGAAAAGAGAGAATTGAAGGCAGCTCTTCA
GTCTCAAGAAAATCTCATACATGAGGCCAGAATACAAAAGGAGAAGTTACAAGAAAAAGTAAAGGCAACT
AACACTCAACATGCTGTAGAAGCTATAAGGTAAATTTAATCTTAAATATTATTCATCTTAGCCACTTAAT
GACTAGGTATTAAGCACTAGGCTTATTTTTATTTTTATTTTTATTTTATTTTCAGATAGTCTCCTGTTGC
CCAGACTGGAGTGCAGTGGTGCAATCTCAGCTCACTGCAACCTCAACCTCCGAGGTTCAAGCCATTCTCC
AGGCTCAGCCTCCCAAGTAGCTGGGACTACAGGTGCATGCTACCACACCTGGCTAGTTTTTTGTATTTTT
AGAGACGGGCATTCATCATGTTGGCTAGGCCGGTCTTAAATTCCTAACCTCAGGTGATCTGCCCACCTCA
GCCTCCCAAAGTGCTGGGATTACAGGTGTGAGCTGCCACGCCCATCCAAGCACTAGTTTTCATATTGCAT
ATTCATAATTTTATGTTTGATCATATTGTGGATTTTCTTAATTGGCTTTAACTTCTGAGTTAGGTTTTTA
AAGTGTTATAAAATTTTGTGTGGTTAATTTTCTTCTGCCACCAGTTAGGGTCCCTAACAGAGACTCTCAT
GTTTAGAAAGTTAGAAACCAAGGTCAGTTTGCATGCTTGGTGAAAATCCAAATGTGCAGGGAATTGTCAT
CCATGTCTCCTGGAGGCCTCACTTTGTTGGTGGAATAAGTTGTGATTAGTAGCTGCTTATCATTAATAGC
AGCCAGCCATTGGGCTAGCCCTCTTGCTTGATTTAAGTCTGATGCACACTTTTATTTTTGCCAGAATTCT
CCTGTAACTTTTCAGTTTTTTTGGTACAGGGTTTCAAGTGTCACTGAAGTGATTTTGCCATTTTGTCTTT
TTCCCTTGAAAAAAGGCCAAATCCCAGTTAAGAAGGGAGTAAATTGCTACTTTTTGAAGTATCAGCATCT
CTTGACTCTTTCTTTCCTCTCCCAATATTCTTTTTATAAAGCAGGAGACAAACTTGAAGAGTTCCAAAGA
GGTTACCAAAAATGATGGGGAAACACAGACTAGATGAGCATTATTTTTGTTTGCAGAGATAAAGGCGTTA
ACAATCATCCTATGTCTTTCCATAGGCCACGGGAAGAATCTCTGGCAGAAAAGAAGTACACCTCTCAAGG
GCAGGGGGACTTAGACAGTGTGCTCTCCCAGTTGAATTTTACCCATACTAGTGAGGACCTTCTGCAGGCA
GAGGTGACTTGTCTTGAAGGCAGGTACATAATTATACACACATTTCAAAAATTTCAAGTTGTTAAAATGA
ATTTTTGATCATCATATTGTAAATGAATGTAATAGTCATAGTAAAAAAAATAAGATTTTTTTCTAGTATC
CACAAATGGCCTAAGTTCTTATTTCTTTTTTTTTTTTTTTGAGACGGAGTCCGCTCTGTCACCCAGACTG
GAGTGCAATGGCACAATCTCAGCTCACTGCAACCTCCGCCTCCCGGTTCAATTGATTCTTCTGCCCCAGC
CTCCTGAGTAGCTGAGATTACAGGCGTGCACCACCATGCCTGGCTAATTTTTATATTTTTGTAGAGACGG
TGTTTCACCATGTTGGCCAGGCTGGTCTTGAACTCCTGACCTCAGGTGATCCACCTGCCTTGGCCTCCCA
AAGTGCTGGGATTACAGGCATGAGTCACCGCACCCTGCCCTAAGTTCTTATTTCATTGAGAAGACAGAAG
CAATTAAAGGAGAACCTCTTAATCTTTCTGTCATCAAATCTACCAGTCTGCCCATACCTGTGCCTTTCTG
CACTTCTAAGACCAACCACTCCACTTGTGTCCTTTTTCTGTAGTTATATCCTTTCTCTTCTGCATCATCA
AGTTCTTTCTGTCTGCTGAATCTATCCACTGGTATAAAATACGCTGTAACATCACCCATCTGGGGGAAAA
ACCATCCCTTAATCCTATGTCTCCGTCAAGTTATTTCCACATTTCTGTACTTCTCTTTAAGACCAAATTC
TTCAAAAGAGTTGTCTATACTGACTCTACTTCTTTACCTTTCACCTGTTTAGCAGCCCTTCACTTAGGTT
TTCCTCCTTGCCACCTCAGAAGCAGGGTAGGAAGGGGTAACAGTTGACACATGGTGATCCTTTTTGTTCT
GCTGTGGTGTTGTTACTTCCTTGGGTGTTAATTGAATCCTGACTAATGCTATACCATACCCACCTAAACA
CTTGTCATTGTCGTACTTATAGAATTTTTTTTGGTTTTTCATAACCTCTCAACTTTTTGATTGGGAAGAG
TGTGATATCTTGTTTTAAACACATGAATCATGATCATTTGTACACTGACATTTTCTGTATGTAAAAGATA
AAATTGCTTTTAACATGAACTGTTAACCTTTAGGCAAACAACAAGAGAAATATCATTTTACACTTTTTAG
GTTATCTTTAAGTAATACATTTTTTGTTTTTACATTTATTTCTGGTTTTGGAAACCAGTAAGTATTTTCT
TACTTTTATCTACATGTTAAATACACAAACATGCATACACACACATATTTTCTGCTTTATTCCTTTTGGA
ATACTTGGAGTACTTGAGATAGTCTTTGTTATTATTGAAGTATATGTGTACTCTTCCGTTTGACTAAAAT
TAAGTTTATTATTTTTGACGTTGGGACAACAGTTTACATTTTTATCACAGTTTTTTTATTGATTGGCTTT
TTAGTAATATTTTAAATGACTGAGGTGATGTTTAAGATATTGGCTGGATATTTTTTGGATATCAAGAATG
TAAATTAGATAGTCCAATTTATAGGTTTCAGGATTTTGGTTTTAAGTATGGGATCTTATTTTTGTCATAC
AGTTTGGAATCTGTGAGTGCAACGTGTAAACAGCTGAGCCAAGAACTAATGGAAAAATATGAAGAACTGA
AGAGGATGGAAGCACATAACAATGAATACAAAGCAGAGATTAAGAAGGTAAAAATCTGCATACCTAGGAT
TGCAAAATTGTATGTGTTTATGGACAAAGCTGTGGATTGGGAGATTATAGGGGTTATTTTTAATGAGAAA
AAAATGTAGTTCAAGGGACCACTTGATATTTAGGAGTGGAAGAGTTTGTGGAATGTGCATAGGGAAAGTG
CCTTCTTTCTTTGGCATCGCCCCTGATGCCCAATAAATATTTTTAAGATAATTGAAGGCTGTCCCCTTTG
AACTCAAAAACTTTATTTTAAACATGTTTTGATGAAAAGTTTTCTAAAAGACATACACTCTGTTGCTTGC
AAAAAAATATGAACTGTCACTTAATATTTTCTTATTGCCTAAAGATAGTATTTATGTATTGTCTCCTGTG
CTATGATGTAGTTAGTATCCTTGTCAGCTATTGAGGTATGCAGAGCTGTTTGTGTCTATATGAATGGGCC
CTAACTGTCCTATTTGTTTGATTTGTATTTTTTGTTACTTCTTTAAGATTACTTCTAGTTCCTTACTTCC
TCTTGTTCTATTTGTTATCCTGTGGACTAAGATACCATATGAGCAAAACTTTTAGATTCACAACTAAAAA
AAAGAATGAAGGTCACAAGTACTCTCCTCTAAATTGTGTGCATGGACCTTGATTCTGGTCCTCAGCACTT
GAATAACCCATCTCTCCCTGTTTTGAGGTATAATAAATGATTGTAGGTATAACCACCTGACCTTCAGTAC
AGATATGCAACTTGGTAAATTGCTAATTTTTCTGCAGATTTAATTTAAGAGAAATTTAATGTCTTGAGAT
AAGGTGAACTTTTATTTTATTAGCTAGTAACATATTATTCTATCTAAATGCTTTCTCTTAACTTTTGGTG
CTTTCTTTTCTGCTTCTTTATAGTTGAAAGAACAGATTTTACAGGGTGAACAAAGTTACAGTTCTGCACT
AGAAGGAATGAAGATGGAAATCTCCCATCTAACTCAGGAGTTACATCAGCGAGATATCACTATTGCTTCC
ACCAAAGGTTCTTCCTCAGACATGGAAAAGCGACTCAGAGCAGAGATGCAAAAGGCAGAAGACAAAGCAG
TAGAGCATAAGGTGAAGCCTGAACAAAACCTTTTTTAAATTTGAAATTTGTTTTAAAGATGGAGTTGATT
AAAGACATAATTTTCATAATATTTAATTCAGTGAATTTTTATTAGATACCTGCTTTGAGCTGAGAGTATG
CTGGCTAGCAAAGAGTTTAGTGAGCATGGTGGGGAGGTGAGAGTAGACACAGACAAGTAAAGTGACTGTA
TCAGTGGTGTATGGTGAGAGTCACTAGAGATCACAAGCAGCCTGAATCTAGACCACTCGCTGCAAAGGCC
TTCCAGAAGGTGTGATAGCAAAGCTAAGTTAGGAGCAAGCTGGGTGATGGGGAGGAAAAGTGTTTCAGGC
ACAGGAAAATACAGAGGTCTCTAAGAGAGAGTAGAAACACGCTTGCTGTGGTTGAAACTAGAGTACAAGT
GGGATGAGTGGAAGGTATGAGACTGGAAAGCAGGGACTGGCTGCTGAAGGGCTACGTTAGCCACGTAAAG
GAGTTATACTGTGACCTTGAGGTCAGTAGGGAATGGCTGCAAGCAGGAGTAAAATTCAGTGGTCCTTAAA
TGGAGAATAAGCAGGGAGACAGAGCACCCAGATGCTGTTACAGTGGTAAGATGGTGGCCTGAGCAGTGGG
CAAGGGAGGCAGAGGGAAATGGATGAATTCTGGAGATACTGGGCTGGTAAAAATGACTGGGCATTGTATT
TGATTGGCAGGGAAGGTGAGGTAGGCAGAGTTAAGGAATGGTGCTCCACAGGATTTAAACTTAGGCAACT
GATGGAAAGTGATGCTTTTTAATGAAATAGAGAAAAGACAGGGAGCAAGTTTGGTGAAGGATGATGAATT
TGTGTGAAAAGTACACAGACTGGGGAAACTGGTGGAGAGGACATTGGTTTGACACATTTGTTTGGAATAA
AGGCTGGAATTTTGTATGGGTAATAGTCAATTCCACAGAAGGCCTGGAGTTGAAGATTAAAATCAGGATT
AGGCATTTAGCTGTGGCTGTCCTTTCTTCATGGTGACATTTGAAACCCCCTGAGAGACTTAATGATTATA
GTGAGAAAGCATAGCGTAAGAAGAGAAGTGACTCAAACCAAACTTAGGATACTTCTCATTTAGTGTAAAA
AAGATGTTGGAATGAAGCTTAAAAAAAGGAGCAGTGAGGAGGGAAATCAGATAGTTTTTGCCAAGCCAAG
GAAAGAAGGAATATTTTCAAATGGGAATGGTCAAGCACAAAAGCTGAGGGGAATGAGAACTGAATATAAG
TTGTTATTTTGTGAATAAAGTACAATTCCATGTGACGTTGCAGGTAGAAGCCAAATTGGAGGAGGCCAGC
ACAGGAGAATGGGTAGGGATTTAAGGGGTTCAGTGGATTGAAACTTTGCTGCTTTGTCACCTGATTTATC
CTATTAGAATGATATCTTCATCCCATTCATGTTCATTCACCTTAATAACATCTTAAAAATCTTTTTAGAA
AGTCCATATATATATATATATATATATATATATATACACACACACACGTACATATATATATGTATATATA
TATATAAATATGTATATGTATATATATATAAATGAAGAATATGACTTAGCCTAATGAAAATTCCATGTGG
AAAAATTTTGTAAGATGCATGTGATTTACATGTTATATTTATTTTTTTCTGTTTTCCCCTTTTCAGGAGA
TTTTGGATCAGCTGGAGTCACTCAAATTAGAAAATCGTCATCTTTCTGAAATGGTGATGAAATTGGAATT
GGGTTTACATGAGGTACATAAATAGAAACTTAAGTTTTTCTACTATCCAAGCCTTCAAAAAAATTACTTT
GTAAATATTTACATTATAAAGAGAGAATTAAGATCAACTTAGATCCTTTTGCAGAAATTTTCTCAAAGAA
AATGAAGGTAAAACAATACTGGAAGTGAGCAAAATATGACTCTCCACTCCAGCAGCAGGAAGGGTTTTTG
TTTGTTTTTTTGAGACGGGGTTTCACTCTTGTTACCCAGGCTGGAGTGCAATGGCGTGATCTTGGCTCAC
CGCAGCCTCCACCTCCCGGGTTCAAATGATGCTCCTGCCTCAGCCTCCTGAGTAACTGGGATCACAGGCA
TGCGCCACCACACCTGGCTGATGTTGTATTTTCAGTAGAGGCAGGGTTTCTCCATGTTGGTCAGGCTGGT
CTCGAACTCCCAATCTCAGGTGATCTGCCCGCCTCAGCCTCCCAAAGTGCTGGGATTACAGGCATGAGCC
ACCGTGCTCGGCCAGGAAGGTATTTTTTTATATTATGTATGTGATAAAATTTACCTACAGTCTGTGTTCT
TAGTTGGAAGAATGTATATGAAGGACTTAGCATATAGTACATAGTACCTGACACTTAGTAAGTATGCAAT
ACATCCTAGATGGTGGTATTATTATTATTATTATTATTGTTAATGATTATTATGTTTGTTATCTTTTTAG
AAAGTATATTGTCTTTAAAATAATTTAATTTTAATTTTATTTCTACAAGATATTCACATAATAAAATATT
TTAGAATCTTTTATATCCAAGGACTTTTAGTAGGTGCTATAATGAACACAGGCTGAATAAGATAGCATTC
TTGCCTTTAAGAGAGATTTTGGATCAGCTGCAATTACTCAAATTACTCTAGCCTTTAAGAGACTAGAGTA
ATCTAGTAGCAAGGATAAGAAATGCATACAAGCTTCTGTGAAACCAGAGAGAACATGATTAGCCAACAGT
GAATTACAGTATAGATGCTCTTTTAACTGTTTTGTTACAAAATTCCTATAATTTGCAAAATGACTCATCT
ATTCTGACCCATCTTTTCTTGGTGTAGGTCAGATGTCTGAAAATTGTATAAATGCTTTTTTCCTGCATTT
CCAGAGAGTATTCCTCCAAAAAGATATATGGTTCCAACATGCTCCCTGTTTGCTCTGAAAAATTCTAATT
TCACAATCCAGGATTGTGGATACTGGGGTGGAAGAACATGCAAAAGACTATTATAGAGTACCTTATTTCA
ACTGGAAAAATGGGGGACTATTTTTGTAATCTTTCAGAAGGGTAAGATTCTCCAAACATCATGACGCCAT
AGAGGATTTTACAACATAAAAATGTGAAGTTTCTGGATGGTAAAAGGCCCTATTAAAAAAGACTAATAGG
GAGAAAACAGTTTATAATCAGACAAAAGGTTAATGTAAATTATTAATATATAAAAATTCCCTACAAAGAT
AATAAGGAAAAGATAATCTAGTAGAAAAATAGGCAAAGAATGTAGATAGATACCTCACAGAATTAATATA
AATATCTAGTAAACAAAGAAAACTGTTCAACCTCTCTAATAAAGCAAAATAGACATTCTTTTACTAATTG
GTTTGATAATAACCTTTAAAAACTAATATATAGCCAGTGTTAGAAAGATGTAAGGAAACAGACTTATTGC
ACTGTCAATGAGAAAGTGAATTGATACGGCCTTTCTAGAGGACAGCTTAGCAATATCCATCAAATGCGTA
AATACGTATAGCCTCTAAATTCCTGGGTCATGTGTGTTTCTGATTTTGATAGTCCTTGCTGAATTGTTTT
CAGAATGGTCTTATCTTTTTACATTCCCATGGAAACCATTCTCTAGGAGCAAGTGTGCATAAAATGGTCA
TTGAAGTATTGTTCGCAATATCCAAAATTTTGAAATAAACTATATTTATTTCAATGGTTAATTTTTGATT
TTATGAATATTACACTGTCATTGAAGGTGGCAGATGAAAACACTCATCTGATCTTGCTCTATCCTCAAAT
CCCTCTAAAACTGTCATGAAAGGATTTATTTTTGTTCTATTTTATTTTTATTTTTTTCCTTTTATTATTA
TACTTTAAGTTTTAGGGTACATGTGCACATTGTGCAGGTTAGTTACCTATGTATACATGTGCCATGCTGG
TGCACTGCACCCACTAACTCGTTATCTAGCATTAGGTATATCTCCCAATGCTATCCCTCCCCCCTTCCCC
CACCCCACAACAGTCCCCAGAGTGTGATGTTCCCCTTCCTGTGTCCATGTGATCTCATTGTTCAATTCCC
ACCTATGAGTGAGAATATGCAGTGTTTGGTTTTTTGTTCTTGTGATAGTTTACTGAGAATGATGATTTCC
AATTTCATCCATGTCCCTACAAAGGACATGAACTCATCATTTTTTATGGCTGCATAGTATTCCATGGTGT
ATATGTGCCACATTTTCTTAACCCAGTCTATCATTGTTGGACATTTGGGTTGCTTCCAAGTCTTTGCTAT
TGTGAATAGTGCCGCAATAAACATACGTGTGCATGTGTCTTTATAGCAGCATGATTTATAGTCCTTTGGG
TATATACCCAGTAATGGGATGGCTGGGTCAAATGGTATTTCTAGTTCTAGATCCCTGAGGAATCGCCACA
CTGACTTCCACAATGGTTGAACTACTTTATAGTCCCACCAACAGTGTAAAAGTGTTCCTATTTCTCCACA
TCCTCTCCAGCACCTGTTGTTTCCTGACTTTTTAATGACTGCCATTCTAACTGGTGTGAGATGGTATCTC
ATTGTGGTTTTGATTTGCACTTCTCTGATGGCCAGTGATGATGAGCATTTTTTCATGTGTTTTTTGGCTG
CATAAATGTCTTCTTTTGAGAAGTGTCTGTTCATGTCCTTTGCCCACTTTTTGATGGGGTTGTTTGTTTT
TTTCTTGTAAATTTGTTTGAGTTCATTGTAGATTCTGGATATTAGCCCTTTGTCAGATGAGTAGGTTGCC
AAAGACAAAAACCACATGATTATCTCAATAGATGCAGAAAAGGCCTTTGACAAAATTCAACAACCCTTCA
TGCTAAAAACTCTCAATAAATTAGGTATTGATGGGACGTATTTCAAAATAATAAGAGCTATCTATGACAA
ACCCACAGCCAATATCATACTGAATGGGCAAAAACTGGAAGCATTCCCTTTGAAAACTGGCACAAGACAG
GGATGCCCTCTCTCATCACTCCTATTCAACATACTGTTGGAAGTTCTGGCCAGGGCAATTAGGCAGGAGA
AGGAAATAAAGGGTATTCAGTTAGGAAAAGAGGAAGTCAAATTGTTCCTCTTTGCAGACGACATGATTGT
ATATCTAGAAAACCCCATTGTCTCAGCCCAAAATCTCCTTAAGCTGATAAGCAACTTCAGCAAAGTCTCA
GGATACAAAATCAATGTACAAAAATCACAAGCATTCTTATACACCAGCAACAGACAAACAGAGAGCCAAA
TCATGAGTGAACTCCCATTCACAATTGCTTCAAAGAGAATAAAATACCTAGGAATCCAACTTACAAGGGA
TGTGAAGGACCTCTTCAAGGAGAACTACAAACCACTGCTCAAGGAAATAAAACAGGATACAAACAAATGG
AAGAACATCCCATGCTCATGGGTAGGAAGAATCAATATCGTGAAAATGGCTATACTGCCCAAGGTAATTT
ACAGATTCAATGCCATCCCCATCAAGCTACCAATGCCTTTCTTCACAGAATTGGAAAAAACTACTTTAAA
GTTCATATGGAACCAAAAAAGAGCCCGCATCGCCAAGTCAATCCTAAGCAAAAAGAACAAAGCTGGAGGC
ATCACCCTACTTGACTTCAAACTATACTACAAGGCTACAGTAACCAAAACAGCATGGTACTGGTACCAAA
AAAGAGATATAGATCAATGGAACAGAACAGAGCCCTCAGAAATAATGCCGCATATCTACAACTATCTGAT
CTTTGACAAACCTGAGAAAAACAAGCAATGGGGAAAGGATTCCCTATTTAATAAATGGTGCTGGGAAAAC
TGGCTAGCCATATGTAGAAAGCTGAAACTGGATCCCTTCCTTACACCTTATACAAAAATCAATTCAAGAT
GGATTAAAGACTTAAACGTTAGACCTAAAACCATAAAAACCCTAGAAGAAAACCTAGGCAATACCATTCA
GGACATAGGCATGGGCAAGGACTTCATGTCTAAAACACCAAAAGCAATGGCAACAAAAGCCAAAATTGAC
AAATGGGATCTCATTAAACTAAAGAGCTTCTGTACAGCAAAAGAAACTACCATCAGAGTGAACAGGCAAC
CTACAAAATGGGAGAAAATTTTCGAAAGGATTTATTTTTAAAGCGCAAACCCACGAGGTTGGGAATAATA
GGAGAGGAGACAATAGCTAAACATTTTGTAAGGGGGAAAGTGGGAAGGAGGGAAGGAAGGAAGGAAGGGA
GGGAGGGAACTTGATATCCCCTAATAAAGTCAATACTAAGCTAGTAGTGGAGAACTCTAAGAACTAAACT
GATTTACACCACAGAATTCTGTAAAGGCTTAGGATTCAGCAGTACTTGGTTGAGAGACTCTTATCTTATG
CAGTGATTATCTGTTCATATTTAAGAATTGAGAACTCTCAGGGGAACTTAAGCCAGACCATGACCAGAGA
TGCTGTTCAGCTTAATTTCTCCTGAAACTTCCAGCTATCAATAATAGTGGTAATACTGTTCTTTCTTTAG
ACAGCTAGATGTGTGCTCGCTACTCAGTTTTCATATTACCTTTCATATTCATTTATCTGTAGTAATTGTT
TATAAAATAATAACTTAGAGAAATTTTTAAAACATACCTGAATGAAGGTAAGCATTATCATTTTTTAATA
AGTATGTTTAATGACATCAGACATCAAACTGGTGAAAACCTTGTCAGCAGTTTAAGCTTTCTCTAGTTTT
GAAATTACATACAGTGTGAATATGTTGTACATATTATGAAGAATAATTATTAGAAAATGCCCTTTTGTAT
TTTACCATGTAGGAAGCTGAAGTTACTTGTACAAATGAATGTTTGGGTTAAATGTCTATTTTAATAAATT
ACCTAATTATCTTAATAATGAAAGCAACAGCTCTGATAAATTACTGACCATGTTTTAAAAACCGATAATA
GTTTCATTTTAACTCGTATGTCCTGCAGATGTTCTTCTCTGATATTTTATCTGGCATTGACAACTTGTTG
AAAGATTTTGTAGCTCATAGTCACTACTTGTACACAGATAGTTCACAGGTATATTTTAAAGTTTATGTTT
TTGTTAGGAAAATTATGTTTGTCAGACCACTCTTGACCAGCTTACTTTCATAATTTGTTTTTTTTTTTTT
TTTTTTTTTTTTTTTACAGAAAAGACCAAGCTAACCATGGTATTAAACTCCAAAGATTACCATAACTTTG
CATAATACTAATTAAAATATTTTGTCATTAGAAAAAAAATCACCCAGACTACCATTTTTTCAAGTAGTGT
CTTACCAGGAAACACCTAGAATTGAAGAGCAATAAAATTTATGATACCAGATCAGAATAGTAATTCCCAC
TATATACTTGAATCCTGCTTTATCATGTCCAAATTTATATATGAGTAGATACCAAAGTTGTAGAAAAGTG
TTTAAGATTAGAGATTATTTATTTAAAAGTTGTCCCTGCCAGCCAGAAGATTTTTCTCACAACTTTTTGT
TCAAATTTCTTTCCTTGAACTAGGTCTCCGTTTTCTTTCATTAGTATTCTGGGGCTATTCAACCTTCACC
TTCTTGCAGAAGAAACATGGCTGCTATTTTCCTTCTTCTTCCCTGTTTCTGATTCCCTCTTACAGCTTTC
CTCCATCCTGATTCCATCCTTCCTCCACCTTCTTGGAATTCCTTGCCACACAAGGAAGCCCGTTGTGCTT
CTAGTGTTCCACTACCATTTACTTGAAGAATTCTTTATCTCTGTTAGTCTTAGGAGGCCTAAAAACAAAG
CTTCATTAAAGCCATAGATTAACTACTTACAACTAAAAAACACTGAATTTTCCAACCAGCAGTATCACAG
ATCACAGGTATTCTTGTACAGATTAAGTGACTCTAGTATTCTTTTTTATTAGGCAAAAGAGATTTCACTA
GCAGACCTCCAGGAGAATTATATTGAGGCATTAAATAAATTAGTGTCTGAAAATCAACAACTACAGAAAG
ATTTGATGAATACCAAATCTCAGCTGGAGATTTCTACTCAGATGTGCAAAAAACAAAATGACAGGATCTT
TAAACCAACACACAGCAGAACAACTGAGTTCAAGAATACAGAGTTCAAGTAAAATTTTTTAAAAGTTTAT
TTAAAATGTGTATTGGTACAATATAATTCTCATTTCTTTGAGAATAATTTTTGACTTACAGAAATTAAAT
CTTCATCAAAGGACTCTATGTTTAATTCTGAAGTATAGTGTGCACAAGTAAAGCTATGATCATGTTAAAA
AATGGCTGGGGGAGGTGTATAGCATTTTCATTAAAGCTTTAGTTTTGTACACTCTGGATATCAGTGTAAA
CAAGATGTGTATTACCTGAGGCTTTTCATGCTGCCTTCCACCTCTTCAACCCCATTTGCCCATAAGAAGT
AGAATTTGGCTTTTTATAAACAGCTTCCTTTGTCTTTTTAGTCAATTTGTTATAGGCTGTTACCGTAAGA
AATAAGCATTCGGTGAAAAGCCACTTGGCTTCAGCTCATACTATATGGCTTACATGTTTTCCTACCCAAA
GCCTTTGCTGACACCCTCACCTCCACTCCTACACCCACCCAATCCCCTAGTTAGGTTAGGTGCCTTCTTA
TGTGCACAGCACTTCTCCTGTCCCAGCACTAACCACACTAGATTACTCTGGCAGCTTGTTTGTCTTTCTT
GCTATACCATAAGTGTGTGAGGGCAGGGACTTGGTAGTTGGAATTGGATATTTGGGGCCTGAATGAATGA
AAGAAATCTCTTTCTGATCTGAGTAGGTTGCTCATTTTGTAAATTGTAGCCTTATTAGTTAGGCTATTAA
GCAAATTTCCTACATTTCTGTTGGAAAGAGAAAAGTTGCTACAATTTTTTATTTGTTTTGTTTTCTAATC
TACCATCCAGGCCAACCCATGGCCAGCACAGACATGATGGAATAAAGACTGAGCACTACAAAACAGATCT
TCATTCTCCAAGAGGACAAGCGTCGGATAGTATAAACCCCATGTCTAGGGTGCTAAGCCCCCTGAGTCCT
CAAATCAGCCCTTGCAGCTCCACCAGGTCTTTGACTTCCTACTCTCTATGTAAAACTCATTCTTTGCCTT
CAGCGCTAGATACAAATGAAGCCAATTTTTCTGACACTATGTCTGAGAGTATGAATGACCAAGAAGAGTT
TATATCTTCGGTATGGAAACTTTCTGATCTTAGTAATTTGTTAGTTTTTTAAGTTTGCTTTGATTTTTTA
TTTTAAGGGTGAAGAAGGTGATTTTTTTTTCCTTACTGATTCTTTTATCATAAGTACTCTTCATTATTCT
TAGGTATCCCTGTCATACAGTTTTTGCTTGTGAAGACACTTTCGGCCAAGACTACTTTTCAAAATGTTCT
GAAATATGTTATACGTATTCATTTGTCTTTAACCCTTATTGGAAATTCCCTTCTTGCCCTTTTCAGTGAT
GCTCAATTTTCTTCTGGAATGGTTAGTTCAAGAACTGTCTCTGGATGCCAAGATAGTCCTGAGGCACAGG
CAGTGAAGGAGGAAAAGAGGAACTGCTGGAAAGGAGAAAGGGAGTGTGGAGGGGGAGGAAGGCTGTGCCT
CTGATGTGTGCAGAGCATGGCTTGGGGGTCACAGATGGTGGCCCTCAGGGCAGAATGTGCCTTGGACACC
ATGCCGGTCCCTTGCTCCTTCCATTGCTGGGCAGGCTGCACCATCTTTTTGCCTTCTGCACCAAAGCACT
TGCCATGGGCCCTTCATTTCCTGTCACCAGCTGTGGCCATATAGATGTGAAAGTGCAGTATTTCTGACTT
ATTAATGTGCTACCAAGTCCATGGAAAGGTTTTCTTTTATTTTTCAAAATAAGTCCTCACTTCACTTTCT
TCCTAAGTTTAGCAGCAAAATTTTTTTGGCACCAGAACTTTCCCTGGCCTGAACTGATGTGTTAGTATGT
ATAGACTTTCTCTATTCCACTTAGTGTGAGTATCCATGTGTTTTGCTTCAGAAATATCAGTGTGTTTGGT
GATGGGATGTTCCCTCAGACTCCACTGGGGTAATACAGTTGATCCTCATTATTCACAGATTTTGTATTTG
TGAATTTGTCTACTCCCTAAACTTTATCTGTAACTCCCAAATCAGTATGCGCAGCACTTCCACAGTAATT
CGTGGACATATGCAGAGTGACAAAAAATTTGAGTGACCGACTTGCACAATCCCAGCCAAGGTCGAAGTAG
GCAGCACTCTGCCTTGTTGCCCCTGCTTATGCGGAGGTGACCATAGGGTGGAGATGGACGGGACAGCGCA
GTGTAGGGAAAGAAGCTCAGCTCCATGCCAGCTGGATAGAGTCTGAATCCCAGCTTCAGCACCAGTTAGT
GGGGCAGCCTCAGGAGAGTTACCACTTCTGAACCTCATGTCTCTCATGTGAAATAAAAAATACAGAATCT
ACCAGAATGATTTGTTCTAGGATTTAGGATTATGCTCTCTGTGTGGGGTATGTGTGTATGTGTGTGTGTG
TGTATTTGTTGTATGTGTGTATTTTCCCTGGGCCAAAAATTCTGTGTTTGCTAATTCAGTGTTTTGGGTG
ACTTTATAGAACACAACTACTGCTATTGTGAATGAGTATTGACTGCATATGGCATATGTTCTATGTTACC
TTTCTAAAATCAGAATTCTGAAATATCACTAGCCTCAAGGGTTTCAGATAAGGGACTGTGGACTGAAAAT
TTGGAGGAGAAAAAATAATTGATTTTAGCTGGCATCTAATTTATTTTTGTTAAATTTTTGTGCTTTAAAA
TAAAAGTGACATACTTAGGTTTTTAAAGTGAGTTTGAAATAATAGGCATTAACTATGGAAGCATATATAA
GTCTAAATTGAAACATTCTTCAGACCAATAACTAATATATGATACTTTACATTGTTACAATTTGGGTCAG
AAACGGAGAGATCTGTGTTAATTTTTGCAGAACTTACTGATTTTTGTTCTTTTATTGCTCTTGTCATTAG
GATGAAAACCAAAAAAGTATCTTCAGAGGAAAAAAATCACCTTTTAATATTGCTAGTTAGAAAATGTCAT
GAACAGTGTATCTTAGAAATATAATATTCTACTTATTTACACATGTCAAAATTTGTGTCTCTTCCAGTGT
TCCTTGCCTGTATCTCCCCTTGGTTCAATAGCTACCAGATTTTTGGAAGAGGAGGAACTGAGGTCTCATC
ACATTCTAGAGCGCTTGGATGCCCATATTGAAGAACTAAAAAGAGAGAGTGAAAAGACAGTGAGACAATT
CACAGCCTTAAAGTAGCCTCTTAAAAAAATCACTATCTTGGAAATAAAAATAAACACCAAAGAGTTACTG
TCATCTGAAGTAGCAGCTCTTTAAAAACATGAAGAGATAAAATTATAAAAATGATACATCTAAAGCAGTG
GTGAAGAAAGCTGAAAAACTGATACTTTTGATAGGCATTTTCTCTGCACTGGTTTGTTTAAAGGACTTCT
TCCAGCAATAAGTTGAAAGAATAAACCACTTTGCTAGACTTTTTTCTCATACGAATATTTATTATCATAA
AGTGATACTTACCTTGCTGACTTAAATGTGAATAGCTATGTACTAATTGAAATAAGGATTTTATGATACA
TGTTGAAAATAAAGTAACTGCAGGAACTTTCTTTAGGGGAAATGTGTAGAAGCATGGATTTAGGGGTCAA
ACATACCTGGATCGATAGACTGGTTTTGCCACTTACCAGCCAACGGGGCTTGTTATTTACTGGGCTGGTA
GCCCCTCCTAGCCAAGGGGCTGGTAGTGTGTAAAGTCAGGCTGGTAGTGAATAAGGGGGGGGTGTGCTAA
AGAACCTTATCAAGCAGTCCTCTCTTGCTCAACACTCATGCAGAGGAGAGAGGCAGGGAGGGCAAGGGAC
TGGCTGTCATGCACGGTGCCCATGGAATATCCATTGGAAATAAATGTTCATCGTCTGCACTGCTGAGGAC
AAGTTTAGATGGGAGACAAAGATCTGGATGTTGATGTGCCGCAGGCATTTGAAACGATGGGAGTTGATAA
GATCCACCAGGGAGGCTGTGGAGAGAGAGAGGAGCAGGAGGCTGGGTTTGGGGCCCTGAAAGATGCCAGC
ATCTGAGGATCACAGGAAATAGATCTAGCAAAGGAACTAGAATGTGCAGTTACAGATGAGAAAGGTCAGT
GTAGTGTACTTGCCAGCTCAAGGGTGTGGCCTCTGTGAACCCTCTTTCTGTAGTGGATCTTTCCCATCAG
CGTAAAACATCCTGTTATTTCCCCTTTTTAAATAAAACCTCAGTTTTTTTTAATGCCTCTCTTTTACTGC
TCTGTATGTTTGCTTCCCTTCACCCAAACACTTCTGGAAAGAGTTGTCTGCAGTGACTCTTTCCAGTTCC
TCACCCCGCCCCACCGTAGTTCTCTTCCTAACCCTCTCCAACACCACTTCCATCCCCAGACTGCTTTCTT
GCTCTTGAGTTCACCAGTGACCTAATCAGTGTCTGAGCATCATTCAGCACAGTTGGACTCTCTCTTGGCT
TCTGTGACCTGGAACTTCTGGTTTCCTCTCTACCTCATCCTCCACTCCATCTCATGGCTTTTCCTCTTCT
AACCTCTAAGTCAGAGGGCCCCCTAGAGCTCTGTCATGGGTTTTCTCCCTCAGCATTCTCTTCCCTAGAA
GTTTCCATTCCATTCTCAAGCTCTTAATACCACCTATAGGCCAGTGACCCTTACATTTATTACTAATCTT
GACCTCTCCCCAGAGCTGCTGTCTTAGATACCTAGCTGCTTATGCGATGTCTTCACTTGGATATCTGACA
AACATCTTGTTTCCAAGAGAGAACTCTTTATTCTCCCTCTTGCTAAGATTCCATATCAGGACATAGCATT
ATGTTTGCCAGTTCCTAGGAGTTACTTATAATTCCTTCCTTACCCACATCCGCATATCCAGTCATTTGGG
AGGTCCTGTCATTTCTTCCCCCAGAATCTTATCTCAGACCCAACCGTTTCCCTCCTGCACTTCTACCACC
TGATCCAGACTACTTCTCTCTCACCCTGATTCTGGCAGTAGCCTCCTAACTGATAGTACTCACACCAGTA
ATTCAGGGAGCTTCTGGAGTTATCCTTTAGAAATGTAAATGAAACAGTGTCACCACTGCTTCCCTTCAGA
CTCTTCCAAGGCTTCTCTTTTTTTTTTTATTGAGACAGAGTCTCACTCTGTCGCCCAAGCTGCAGTACAG
TGGCAGGATCTCGGCTCTCTGCAACCTCCACCTCCCGGGCTCAAGTGATTCTCCTGCCTTAGCTTCCCGA
GTAGCTGGGATTACAGGCGCATGCCACCAGGCCCAGCTAATTTTTGTATTTTTAGTAGGAACAGGGTTTC
ACCATGTTGGTCAGGCTGATCTCGAACTCCTGACCTCAAGTGATCCACCCGCCTGAGCTTCCCAAAGTGC
TGGGTTATAAGCGTGAGCCACTGCACCCAGCCCCAAGGCTTCTTACTGCACTCAGAATAAAATCCAAATG
AATTAGTATAGCCTACAGAGTGTCAATAATCTGGGCCTAATCACCCATTGCATCATCCTTTACTGTGCTC
CACCCATGCTGGCGTCCTTTCTGTTTATTGAGTGCAGCAAGCTTTAACCTGTTCGGGTCTTTCATTTGCA
GTTTACCCTGAATATTCCCTTTCTCTTACCTCACCTTTGATTGGCGTTTTCCCCCTTGCAGACCTGCACT
CTGGCATCCTCTCCTGAGCAAGCCTTCTTTGATTGCTGCCTCCCAAACCTGTTTCTCCCCATTTCTCTAG
CTCATTGGCCTGTGTTACTTGCTTCATAGTGCTCACTACTATCTGAAATCATTTCATTACTTGATTTATG
TTTGTACTGTCTATTTCTGTCTTTAGAATTAAAACTCCTTGAGGGCAAGGACTTTGCTTCTCTGGTTCAT
GGTGGGATCCTCATCACCCAGCACAAGCCCAACACTTAGGAGAGGCTCAGCTAGTTTGAACCAATGGAAA
ATGCCATTCCTGAGGTGCACCACATCGTTTCTTTTGTGTTGGTGTGCATGCTGTCCTTCAGTTTGTCTCC
TCTTCCCACACCCTGTCATGTGCTCCTAAAACTCCCCCTTTACTTGGCTACTTTATCTGGCTTGGCAAAG
CTTTCCCATATCCCCTGACCCATGTCAGGGAACGTTTCTCCTCATTGCTGTCATGGCACCCTGTGTGTGG
CTCTGACCAGACTTTGCTATCCGCTTTGATTTCTGTCTTTCAGGGGCTAAGTCCTGCCTACATCCTCAGT
CAGCATGCTGCCTAACACATAACAGATCCTAAGCAAATATTGGGTGGATGAAAATCACTACCATTCAAGG
CTTCATTGTATTTATCAGTAAAACTGAAATAATATCTAATGGGGTCGAGAAGATTTACTGAAAATATATA
TTTGAAAGGGCTTTGCAGTTTTAAGTACTGTACCTATGTGCATTGCTGTTACATTCAGGAGATTTCTGAG
TTTTAGACAGTCCCAAGCTTCAGCTTAAAATCTGTAGACTGCAGCCCGTTTCTGAAACGTTTGTACTACG
TGTTGACTAGGAGGAACAATGACAGACTCTGTAGTCACCGGAAATACTTAAGGAATCATGAGGTACTATG
TATTTCCTTAAATTATACTGTTTTTTAAAGCTGAAGTATCTAATAGTTAATGGGTTGTCCAAATTTGTCA
GAACATTTGACTGTAATTTAATGTCTCACCATTTTTCAAAAAGATATATTAATACCAAATATTAAAATGT
GTCAAGACTAAATCTGAGTTAGTGTCTTAACACACATTTTCCAAAATCTGTCCGTGATCAGGGTACCCAA
ATGAGATGAAAAGATTAGGGATTTGTAGTCTACATCTGGTGTTTACAGATAATGTTTTATTGGGCAAGTC
ATCTCATTCTTCTTGTCCTGGATTCCCATGAATTTTCATCTGGATTCCTACGCAGATTTTAGCTGAAGAG
GGCAAAGTTGACTTCCCACTGTGGGCTGGACATTACCTTCCCTTGCAGACTCATTTGATCTCAACAAGTG
TAGAAAAAATACTACAACACTCTGTTTCTGTGACATCTGCTTAAACCCAAACAATTCTTACCTTCTCTCT
CTTACATACACATAACACCTGGTAAACTGTGGGTGATGATTTTCTGTTTGCAAAACACTCGAGTCCAGTA
CCTAAAAGTTGCAGATAAGAAAGATAATTATAAAATAAGCTCTATGGCATGAGAACATACGTTAAGCAAA
CAGTTGTCATGAACAGCACTGAAATGTAGAAAAAGTTAGAAATGTGTCAGCTTGTCCCAAAACAGTTTGA
AATAGTATAAATGCATCAAAAAGTAACAAACAGAATCGGGGCAGAGGGAAAATGAGGGCTGTAAACATTG
ATTCACTAATGAATCTAGTTGTCCAGTCCAGTTGCTAGAGGAAGGCAGCCAAGTTACCTCTGAGCTTCAG
AGGGGGAAAAGCAGAGGAGAAAACATTTACCTAAAAGTTTTATGGTGCCCATAAGATAAAAAAAAAAAAA
AATCATAGACTTAAGAAAAGCACAGATTTTTTTTGTGTGTTTGAATAAGCGTTTTGTAAAGTATGTGAGT
ATCCAGAAGAGAAGTCCAACATTTTCATAACTGGTAATAATAATCTGCACAGCTCCCCCCACAGTATGCT
TCACTGTGGGTTGATGGCATAAGGCTGACATCCAAATTCAGAGGAAACTGTTCTGAGAGGGCCACAAACA
TGATTGCGTAAAGCTCTCATGGCCTGAAAAGGGAAAATTTAGAGGGATGATAGACTGCTTTAAACCAATC
TTTTGTAAAAGCTATTTCTCCGCAGTGCAATTTTGATAATTCAGACATACAATTCACATCTTTTTTTTTT
TCCCTCTCTGTGAGGAACTAGAATTTGGGAATTTTATGTTGCCCATTAAGACCAGGTTCAATTATTTGAC
AATCATCCAGTCAGAAGAACTTTTGCACTCCTCTTACAAAAAGAACGTAAGGCAGTAGTTGATAGGGGAA
GGTCTAAAACTCCTTGATATTAAATGCAAAACATTTATTAATGTTAATAATAGTAATATAATAATATTAA
ATGCAAAAAATGTGCATTGTGCATTTTTCTGGGGAGAAGGTTCATAATTTTCATTAGAATCTTGAAGGTG
TCTGTGAATCAAAAAGAGATTAAAAACCATAGCTTTAGGGACTAGAGTTGTCCCAGAATGGTATCCCATT
TCCTGGGGAGAGACATAAAATTGGAGGAGTCCAACCTCTCCCTTCTGGGGCTGAATTTTAAAGTGCAGTT
GAATAGTCATTCAAAATTTAAGCTTTTCAATAATAAAAAGACAATTCAGTTCAAAAATGGACAAAAGATT
GGAATAGACATTTCTCCAAAGATGTACAGATGGCTATTAAGCAAATGAAAAGATGTTCAACATCATTAGT
CATCAGAGAAATGCAAATCAAAATCAAAATAAGATACCATTACACATCCACTATAATAAAAAAAAAATGG
ACAATCACAAGTGTTGAAGATGATGTGGAGAAATTGGAACACTCATATGTTGCTGATGGGAATGTAAAAT
AGTGCAGTCACTCTGGAAGACAGTTTGGCAGTTCCTCAAAAAGAGTTACCATATTACCCAGCAATACCAA
TCCTAAATATATGCTCAAGAAAAATGAAAATATATCCACACAAAAATTTGTACACAAATGATCTTAGCAA
CATTATTCACAATAACACCAAAGTGGAAGCAACCTAAATATCCATCATTGATGAATGAATAAACAAAATG
TATATCCATGTAATGGAATGTTATTTGGGAGCAAAAAGATTTGAATGAAGTATTATACTACTGATGCCAC
AGCATGGATGAACCTTGAAAACAGGAAGAAAGCAAAAAAAAAAAAAATCACATATTGTATGACTCCATTT
ACATGAATTGCCCAGAATTGACAAATCTGTAGAGATAGAAAATAGATTGGTAGTTGCTAGGGATTGGCGG
CGGAAGGTTGGGGTGGGAAATGGGGTGGGGATAGGAGGATTGGGAGGTGAAGACTAAAGGGTATGGGGTT
TCTTTTTGAGATGAAAATGTTCTAAAATTGATTGTGATGGTTGCACAACTCTGAATATAACAACTAAAAA
CAACTGAATTGTGTACTTTAAATGGGTGAATTATATAGTATGTAAATTATATCTTAATCACTGTTACCAA
AAAAAAAAAAAAAAGCCTCTGATGCATGGCAGCTCCTTAAAAATATCAAGTTTATATTAAGGGAATACCA
CAACACATATAGCACAAGCTATTCTAAAGTTTTCTATTTTCAACTCTCAGTTCACAGGAGTAACGGTACC
TTTCTTGGAGAGAGTGGATGTTCAAGCTCTGTTAATCATTGCTTGTGGTGTGGGGTGGCTCTGCACTGTG
CAATATTGTGGGGCAGAGGGCCAGGGGCCTGCACACCGCAGGGGTTCCTGCTCTTCCGGGTGGGAGTTCA
GTCTTGGTGCTGCTTCCTGTTAGGCGAGGTTGCAGGCAGCAAAGGCCACTCATCAGCCAGGCAGTGTCTT
GGCTAAGTGTATTCTAAGGAGAAGCTGCAGGAATGCTTGGCGCTAACCTCAGTGCTTAGAGGAGGACTGT
TTACAAACACTTTAGAGGGTTACAGACCACTGATGTGGAATATGAGCATATTGAAGTTCTGCCCTAAGAC
TGCTTAGCAAGTCCCTCAGTATCTCTACAAGCATGACTGGGCAAAGTAAAGAAAGAAAAGCCAAGTACAT
TACCTAAACCTGCTTTATTTCTTTGGCTATGAGAAACAGATGAGCTAAGATACTCTTTGAAACTAGTCTT
CCTAGTCCTTTTCTGGAGTGGCTATCCCTTTGACAGGCATCCACTGTGGATGGGCTAATCATCAGTTCCC
AGAGCTGTCCTGATTTCTGTGGCACCAGCTTTGGGAGGTAATGGGGGCCTCTCAGTGGGCCAGCTCTTGG
ATTTGTCTTGCCATTGATGTATAGCATAAACCCAAACAGCCCACCAGTCCCTGACTTTTCTGCGACCTAG
GACATGTGTTTCAGATCCCCAACTTAAATAAAATACAACAAACAAGACCGGAAGTGTTATCTCCCAGTTA
GTTTCTTCAGCCACAGGCAAGCATAGCCCTTGGTCCCAACAGGAGCATGCACTGCAACCCTGCAGGTTCC
TGTGTAACCCAACAGGGCAGTGGCTGGGGCAGGAGCTCAGTGCTTCCCCAAGGATCCCCTTTTCTGGAAG
ACAGGCCCCTCAGCCCAGCATGGAACCCCAGCAGTAGCCTGTTCATATTAATAAATTCTGGACCATGGGC
AAAGGACAGGAAAGGTTCCAGATTGGTGATATGTTCAACCTGCTACCTCCTGCATGAGCTATGGCTTGTA
GACTAAGAAGGCATCACCTGATCTGTTTATTTTGCTCCAAAAAGGTTTAACATTAACCCATTGTTTAACA
ACACCAAGCAATTCCAGACGCTTCCCCAGGCAAAGTCATTCCTTAAATCCACATTTCTCAAGCTGTTTTC
CAGAGATCCAAAATATTTGAAGTATTAAGTGTTTTATTAGTCCATTTTCATGCTGCTGATAAAGACATAC
CTGAGACTGGGTGATTTACAAGAGAAAGAGGTGTAATAGACTTACAGTTCCACGTGGCTGAAACCTCACA
ATCATGGCGGAAGGCAAGGAGGAGCAAGTCACATCTTACATGGAGGGCAGCAGGCATAAAGAGATCCTGT
GCAGAGAAACTCCCCTTTTTAAACCATCAGATCTTGCGAGACTTATTCACCATCACGAGAACAGCGTGAG
AAAGACCTGCCCCCATGATTCAACCACCTTCCACCAGATTCCTCCCACAACACGTGGGAATTCAGGTGAA
ATTTGGGTGGGGCCCCAGCCAAACCACATCATTCCACCCCTGGACTCTCACATATCTTGTGTCCTCACAT
TTCAAAACCAATCATGCCTTCCCAACAATCTCCAAAAGTCTTAACTTATTTCAGCATTAACTCAAAAGTC
CATAGTCCAAAGTCTCATCTGAGACAAGGCAAATCTGAGCCGATGAGCCTGTAAAATCGAAAGTGAGTTA
ATTACTTCTCAGATACAATAGGGATACAGGCGTTGGGTAAATATACCCATTCCAAATGGGAGAAATTGGC
CAAAACAAGGGGGCTGCAGGCCCCATGCAAGTCCGAAATCCAGCGGGGCAGTCAAATCTTAAAGCTCCAA
AATGATCTCTTTTGACCCCATGTCTCACATCCAGGTCACGCTGATGCAAAAGGTGGCTTCCCATGGTCTT
GCACAGCTCCACTCCTGTGGCTTTGCATGGTACAGCCTCTCTCCCAGCTGCTTTCATGGGCTGGCATTGA
GAGACTGTGGCTTTTCCAGGTGCACGGTGCAAGCTGTCGGTGGATCTACCGTTCTGGGGTCTGGAGGACA
ATGGCCCTCTTCTCACAGTTCCACCAGGCACTACCCTAGTGGGGACTCTGTGTGGGGGCTCTGACCCCAC
ATTTCCCTTTCGCACTGCCCTAGCAGAGGTTCTGCATGAAGACCCAACCTCTGCAGCAAACTTCTGCCTG
GATATGCAAGCGTTTCTATACACCCTCTGAAATCTAGGTGGAGGTTCCCAAACCTCAATTCTTGTCTTCT
ACACACTTGCAGGACTAACACCACATGGAATCTGCTAAGGTTTGGGACTTGCACCCTCTGAAGCTATGGC
CTGAGCTCTACGTTGGCCACTTTCAGCCACAGCTGGAGTGGCTGGGACACAGGGCACCAAGTTCCTAGGC
TGCACACAGCATGGGGACCCTGGGCCTGGCCCATGAAACCATTTTTTCTTCCTAGGCCTCCAGGCCTGTG
ATGGGAGAGGCTGCTGTGAAGACCTCTGAAATGCCCTGGAGATATTTTCCCCATTGTCTTGGGGATTAAC
ATTCGGCTCCTCATTACTTATGCAAATTTCTGCAGCTGGCTTGAATTTCTCCTCAGAAAGTGGGATTTGC
TGTTCTATTGCATTGTCAGGCTGCAGATTTTCCAAACTTTTATGCTCTGTTTCTCATTTAAAACAATGCT
TTTAACAGCACCCAAGTCACCTGTTGAATGCTTTGCTGCTTAGAAATTTCTTCTGCCAGATACCCTAAAT
CATCTCCCTCAAGTTCAAAATTCCACAAATCTCTGGAGTAGGGGCAAAATGCCACCAGTCTCTGCTAAAA
CATAGCAAGAGCCACCTTTGCTCCAGTTCCCAACAAGTTCCTCATCTCCATCTGAAACCAGATCAGCCTG
GATTTCATTGTCCATATAGTTATCAGCATTTTGGTCAAAGCCATTCAACAAGTCTCTAGGAAGTTCCAAA
CTTTCCCACATTTTCCTGTCTTCTTCTGAGCCCTCCAAGCTGTTCCAACCTCTGCCTGCTACCCAGTTCC
AAAGTCGCTTCCACATTTTCAGGTATCTTTTCAGCAACACCCCACTCTACTGGTACCAATTTACTGTATT
AGTCTGTTTTCATGCTGCTGATAAAGACATGCCTGAGACTGGGCGATTTACAGAAGAGGTGTAATGGACT
TACAGTTCCACTTGGCTGGGAAAGCCTCACAATCATGGCAGAAGGCAAGAAGGAGCAAGTCACATCTTAC
ATGGAGGGCAGCAGGCAAAAAGAGAGCCTGTGCAGAGAAACTCCCCTTGTTAAAAACATCAGATCTCATG
AGACTTATTCATCACAGACTTATTCTGTGCTGTCATGAGAACAGCACAAGAAAGACCTGCCCCCATGATT
CAACCACCTCCCACTGGGTCCCTCCCACAACGTGGGAATTTAAGATGAGATTTGGGCAGAGACACAACCA
AATCACATCAAGTGTTTTTTAAGAATATGCCAAAACAGAATGGAAAACACAGCAAGTCAAATTCCCCAGA
GCCAGGTCAGCCTCTGAATGGGATTAGGAGTGCCCTCTCTCCCTCCCACTGGCAAGCATCTTGCCTCAGC
CTCCTCTGTTGCTTCCCAAATGGAATGGGAGCTGGTGGCTTAGGTACTTAGAGGTTCTTTACCTTCTGTG
TGTCAGGGATGCCTCTGGCAGAGTTGAGGAGCCTATGGACCCCTCAGAATAATGACTAAGTGCATAAAGT
AAGATACAAAGGCCGGGTGTGGTGGCTCACGCCTGTAATCCCAGCGCTTTGGGAGACCGAGGCAGGTGGA
TCATGAGGTCAGGAGATCGAGACCATCCTGACTAACATGGTGAAACCCCGTCTTTACTAAAAATACAAAA
AATTAGCTGGGCATGGTGGCGGGCACCTGTAGTCCCAGCTACTTGGGAGGCTGAGGCAGGAGAATGGCGT
GGACCCAGGAGGCGGAGCTTGCAGTGAGCTGAGATCGTGCCACTGCACTCCAGCCTGGGCAACAGAGCGA
GACTCTGTCTCAACAACAACAACAACAACAGCAACAACAACAAAAGATACAAAGCATTAAATGGAAAATA
AAATATCATCATTCAGTTATGAAAACCTAAGAAAAAACAAATTTGTGATGTAGTATTATATGCACTTCTT
TGTTAACTCATTAAATAACAAGACTGAGTGGCAGGTCTATCACCTACCATGCTTTTGAAGTGGGGACATA
TGAAAGTAATGTTTTAATATGTCCATCACAATTGTAATGAGCTATGAAAATATCTGTGATTTCTATTGGA
ATAAAGCTAAAGGTACTGCTAGCACTGTGGCCTATACTCATAATGGAAGAAAAGTACATTTCAGTCAGAG
GTTATTGAAAATAAGGATGTCAGTTTTTCCCACCCAAGTTCATGGAACCCTAAATTGTATCCTTGGATCC
TTGGGAAGTGTCTGTGGACTCCAGGTTAAGAATTTAGAGGCCTACTTATGTTTTATCTCATGCCTGGTCA
TGTTGGTTCCCTGCGAACATTCACTTGTTTACAGGAGGCAGTATTTGTGCTTAATGTCAGTCTTGACTCT
AAGAACTTAGTCTTTTTTTAAAAAATAATTTCAACTTTTATTTTAAATTCAGGGGGTGCACGTGCAGGTT
TGTTACATGGGTGTACTGCATGATGCTGAGGTTTGGGATATGAATGATCCCAATACCCAGGTAGTGAGCA
TAGTATCCAATAGACAGTTTTTCAACCCTTGCTCCCCTCCCTTCTTCCTCTTTCTATCCCCAGTGTCTAT
TGTTGCCATCTTTATGTCCGTTACTACCCAATGTCTAGCTCTCACTTGTAAGAGAGAACATACAGTATTT
GGTTTTCTGTTCCTGCCTTATCTTGCTTAGGATAATGGCCTCTGGCTGCATCCATGTTGCTGCAAAGGAC
ATGATTTCATTCTTTTCTATGGCTATGTAGTATTCCATGGTGTATATGTACCACATTTTCTTTATCTAAT
CCGCTATTGATGGGCACCTGGGTTGATTCTATGTCTTTGTTATTGTGAATGGTGTAATGCTGAACGTATG
AGTTTATGTGTCTTTTTGGTGGAATGATTTATTTTCTTTTATATATATACCTAGTAATGGAATTGCTGGG
TCAAATGGTAGTTCTGTTTTAAGTTCATTGAGAAATCTCCAAACTGTTTTCCACAGTGGCTGTACTAATT
TATATTCTCACCAACAGTGTATAAAGCATTCCCTTTTCTCTGCGACCACACCAGCATCTGTTGTTTTTTT
GACTTTTTGATAATAGCCATTCTGACTGATAGGAGATGGTTATCTCAGTATGGTTTTCTTTTGCATTTCT
CTGATAATTAGTGATGTTGAGCATTTTTTCATATATTTGTTGACTGCTTGTATGTCTTCTTTTGAGAAGT
GTCTGTTCATCTCTTTTGCTCACTGTTTAATGGGGTTGTTTGCTTTTTGCTTGTTGAGTTGTTTAAGTTC
CTTATAGATTCTGGATATTAAATCTTTGTCAGATTGTTTAGTTTGAGGATATGTTCTCCCATTTTGTTTA
CTCTGTAGAAAGTTTCTTTTGCTGTGCAGAAGTTCTTTAGTTTAATTAGGTCCCACTTGTCAATTTTTGT
TTTTGTTGCCGTTGCTTTTGGGGACTTAGTCATAAATTTTTTGCCTAGGCTGATGTCCAGAATGGTGTTT
CCTAGGCTTTCTTCTAGGTTTCTTATAATCTGAAATTTTACATTTAACTCTTTACTCCATCTTGAGTTAA
TTTTTGTATACAATGAAAGGTAAGGGTCCAGTTTCAATCTTCTGCATATGGTTAGCCAGCTATCCCAGCA
CCGTTTGTTGAATAGGAAGTCCTTTCCCCATGGTTTATTTTTGTTGACTTTGTTGAAGATCAGATGGCTG
TAGGCATGTGGCTTTATTTCTGAGTTTTCTATTCTATTCCATTGGTCTATGTGTCTGTTTTTGCACCTGT
ACCATGCTGTTTTGGTTACTGTAGCCTTGTAGTATAGTTTGAAGTTGGATAGTGTGATGCCTCTGGCTTT
GTTCTTTTTGCTTAGGGTTGCTTTGGGTATTCTGGCTAAGTAAGAGCTTAGCCTTTAAATGGGCTGAGAT
AATAAATCAACTTACCCAAGCAAGGTAATATTAATTTTTTTTATTCATGACATACTTTTTGGAATTTTGG
AGAACTATTGTTCTGCATCTGACAGATCAGAGCATTCTGCCTGTGTTAGAGTTAGGCCCTGAGGTTTTGG
AAGCCCTCAGCATTTAGGACAGCATTTAGGGCAAGCCTGGGATGGTGAGTGCTCTTCTCCTGGGTCTTCT
TGGAAGAAGACCAGGTTTGAACAAGCACACTACAGTCTCTGTAGGTCACAATCCCAATGCTTGACATGCA
GCCTCGTGGGCTGGCTGCATATGATCAGGGAGAAGCTGGACCACTCAGCCATTTCCTCTGCAGAGTTGCA
GGGTATGCTACTGTCCTCTGTGGACAAGGGGCCTATAGCACTATTGGAGGAGCTGGTGCTCTGTGTGCTC
CAAGATAATTGCTCACTGCCGAAGTGCAGCTCCTTAGGACTGGGGTGAGAAGAGAGATGGAGGAGCGGCT
TCTGCAGGGAGACCAGACAGAGCTCAGGTGGGCACAGCTCTAGACAGCAGTCCCAAATCTGTTTTTACAA
AGCAATCCAACAGTTTCTGAGTCTTTCTGAAATCTCAAGATGCATTTTGGATCCTTGACCTAAACTTAAA
CACTACAAACAAGGCCAAAACTATTATCTAGTGATGTGTCAGACAATTATGTCTGTCTGCTGCTGGCATG
CTCTGCTGCCTCTCATGCCAAGCTACAGGGTGCCTGCTCCAACAAGAAATATAATTCTTCAAATTAGCAT
TTCTCATTATTTTTTAACACTGGATCTAAAAATATTTGATTAGATTCAAAATTTATCCATAATATTGCTA
AGATTTTGTTTTTTTGGTTATAGAGTCCCACTCTGTCACCCAGGCTGGAGTTCAGTGGTGCAGTCACGGC
TTACTGCAACCTCCACCTCCTGGGCTCAACTGATCCACCTCAGCCTCCTGAGTAGCTGGGACTTCAGGCA
CGTGCCACCACACCTGGCTAATTTTTTATATTTTTTTTTGTAGAGATGGGGTTTTACCATGTTGTCCAGT
CTGGTTTTGAACTTCGGGATTCAAGCAATCCGCCAGCCTCAGCGTCCCAAAGTGCTGGAATTACAAGTGT
GAGCTACCACCACTGGCAGTTAAGAATTTTAACAATTTGTCAATGAAACAAGAATCTCAATTAGAGTCTT
TATATACAATCTGTACTGTTGGAATTTTCAAATAAATATTGTAAAGAAAATTAACACTATTTTGCCAAAG
CTTCTCATCAATACGGACTATGAAGAATTTAAAGACTGTCACTAAAAACATCAGGAATTTTCATGCTAGA
AAGCTTAAGATAACTCCTTCCTTCCTTCCTTCCTTCATCCCTCCGTCCCTCCCTCCGTCCCCCTTCCCCT
CCCTCCCTGTCTCTCTCTCTCTTCCCTCTTTCCCTCCCTCCCTCCCTCCCTCCCTCCCTCCCTCCCTTCC
TTCCTTCCTTCCTTCCTTCCTTGCTTCCCTCTTTCCCCCTCCCTCCCTTCCTCCCTTTCTTCCTTTCTTT
CCTTTTTTCTTTCTTTCTTTGGAGACAGGGTCTCACTCTATGGCCCTCTTTCTCCCTCCCTCCCTCCCTC
CCTCCCTCCCTTTTTTCCTTTCTTTCCTTTCTTCCTTTCTTTCCTTTTTTCTTTCTTCCTTTGGAGACAT
GGGCTCACTCTGTGGCCCTCTTTCTCCCTCCCTCCCTCCCTCCCTTTCTTCCTTTCTTTCCTTTCTTCCT
TTCTTTCCTTTTTTCTTTCTTTCTTTGGAGACAGGGTCTCACTCTGTGGCCCTCTTCCTCCCTCCCTCCC
TCCCTTTCTTCCTTTCTTTCCTTTTCTCTTTCTTTCTTTGGAGACAGGGTCTCACTCTGTGGCCCAGGTT
GGAGTGCATTGGCATAATCACGGCTCACTGCAGCTTAGGCATCCTGGCCTCAGGTGATCCTCCCACCTTA
GCCTGCTGAGTAGCTGTGATTACAGGTGTCTGCCACCACATCTGGCTAATTTTTTAAATTTTTTGTAGAG
ATGGGGTTTCCCTATGTTGCCCAGGCTGGTCTCGAACTCCTGGGCCCAAGCAATCCACCCTAGGCCTCCC
AAAGTGCTAGGATTACAGGCTTGAGCCACTGTGCTGGGCCATAGGATTTTGATCAAATATTTTTGCAAAG
ATTGTATTAGAAGTTTCATATAAATGCAATATTTCTTAATACTTGCATTTTATTTTCATCATCATAATTA
CATATTGCAGAGAAATGCAATGACTTTTACATGTAAGCCCCCTTAAATTCTCTCCAGTTTACCCCACAGG
TGTAAATATAATTGTTTATGCTTTTCACGGGAAGTCTTCTCTTGAATGTTGGGCCTTTGGATGAAAGAGC
CTTGGTGGTGAAGCCCTGGTATGGTTTCTGGGACATTGCAAGCCAGCCCTTTCTCCTCTTCTTTTATGCT
TCCACTAAAATATTTCTGCAGTTAAAATTTATTTTGATTAATGCTTAAACATACATGGGCTACTCAGAAT
GACAGTTTATCCATTTATTAATTCATTCATCATACATTAATCAAGCAAACCCCTCATCTTAGAAGACTGA
GTTCCTGCTATTCCCTGAAGATGCCAGCACAGAAGACACAGATGCCAGCTCCCTTCCTGCTCCAAGAACC
TCTGACAAGTGCGCATGGCAGTACTAGTAACACACCTGTGCCCTTAGCAATTTGTATCACCAATGCTTGA
AACCCCAGTGCCCAGGGCAGTGAGGCCCAGTGAAATGAGACTGAGAGCGGAGAGGTAGGGCCTCAAAGCC
AGCTTGCCACTTAGCAGCTGTATGCCCTTGGGCAGTTTCTTCATATGCAGAATGTGGACTATGATCCTAA
TTCATCAAGGCTGTCACAAGGCCAGGTGAGATGCTGAAAAGGGAAGACAATAAAAATGCTGTAGATGGAA
CTCCGTATGTAACTTCAGCCACCCAAGAGGGCAGCTGTAGGAAGGCATCAGGGCAGGAAGTTGACCCTGG
CCTCGGGGTCTGGGAGACAATAGAGGGGCACGCGTGGGGTGACTTGGAGAGCCAGCCCATCCAAAGGGAG
GAACTAAAACTTGGCTCCCTGTGTTCACGGGCTTCTCAGGAAAAGCTGGCTGCTCAATTTTTTAATGCCA
TTTTCCTGGTTTTCAAATGTTAGCACCAATTAACTTAAAACAAACAAAAAACAACACTGCGCAGGCCAAA
CAGTAAAGTTTGTAACCTCTGCTCACACTAGTGTTGGCGTAGTAAAGATGTCCTGGGACCATGATGCATT
ACTGCAGTTAAGATTCCCAGGAAAGGGAGTGGGGGCCCAGCAGGAGGAGAGCCATCTCCCCTTGTCTGCA
GGGCTCCAAACAAGACTGGAGCCACAGAAACTGGCCACCCATCATATGTCCCTTCAACCAGAATGGGGCT
GTTTTTGCAATAGAGTGCCATCTATAAAGAACCAAAGCCAAAAAACTTCTAGGAAAAAAAAAATAGGAGA
AAATTGTGACCCTGGGTTAGGCAAAGCTTCTTTTTAAAAAATAGTAGCATGGATGATTTTTTCTCTTGGA
TTTAACAAATGTCAACATTTCATCATATATGCTTCACACTTGTATGGAAAAAAAGCTTACCAAAAACTTT
TTTGTGGCCTTTTCTAATCCACTTTTTTTTTTTTTTTTTTTTTTTGAGATGGAGTCTGGCTGTGTCACCC
AGGCTGGTGCAATCTTGGCTCACTGCAAGCTCTACCTCCCGAGTTCAAGCAATTCTCATGCCTCAGCCTC
CCCAAGTAGTTGGGACTACAAGTGTGCGCCACCATGCCTGGCTAATTTTTTATTTTTAACTTTTTTTTTC
TTACTTTGAGTTCTGGAATACATGTGCAGAATGTGCAGGTTTGTTACACAGGTATACATGTGCCATGGTG
GTTTGCCGCACCTGTCAACCCATCATCTAGGTTTTAAGCCCCAAATGCATTAGCTGTTTGTCCTAATGCT
CTCCCTCATCTTTCCGTCCACCCCCGCGACAGGCCTCAGTGTGTGTTGTTCCCCTCCCTGTGTCCCTGTG
TTCTCATTGTTCAACTCCCACATATGAGTGAGAACATGCAGTGTTTGGTTTTCTGTTCCTGTGTTAGTTT
GCTGAGGATGATGGCTTCTGGCTTCATCTGTGTCCCTGCAAAGGACGTGATCTCATTCCTTTTCATGGCT
GCATAGTATTCCATGGTGTATATGTGCCACATTTTCTTTATCCAGTCTATCATTGATGGGCATTTGGGTT
GGTTCCATGTCTTTGCTATTGTAAACAGTGCTGCAATAAACGTGTGTGCATGTGTCTTTATAGTAGAATG
ATTTAAATTCCTTTGAGCGTATACCTAGTAATGGGATTGCTAGGTCAAATGGTATTTCTGGTTCTAGATC
CTTGAGGAATCACCATGCTATCTTCCACAATGGTTGAACTAATTTACATTACCACCAACAGTGTAAAAGC
GTTCCTATTTCTCCACAGCCTCGCTAGCATCTATTGTTTCTTGACTTTTTAATAATCGTCATTCTGACTT
GTGTTTTTTTTTTTTTTTTTTTTTTTTTGAGATGGAGTCTTGCTCTGGTCACCCAGGCTGGAGTGCAGTG
GTGCGATCTCACCTCACTGCAACCTCTGCCTCCTGGGTTCAGATGATTCTCCTGCCTCAGCCTCTTGAGT
AGCTGGACCTACAGGCATGCACCACCATGCCTGGCTAATTTTTGTATTTTTGTAGAGATGGAGTTTCACC
ATGTTGACCAGGTTGGTCTTGAATTCCTGATCTCAAGTGATCTGCCCACCTTGGCCTCCCAAAGTACTGG
GGTTACAGGCGTGAGCCACCATGCCCAGCCCTCACTGTGATTGATTTGCATTTCTTTAATGATCAGTGAT
GCTGAGCTTTTTTTCATGTTTGCTGGCTGCATAAATGTCTTCTTTTGAGAAGTGTCTGTTCATATTCTTT
CCCCACTTTTTGATGGGGTTGTTTTTTTCTTGTAAATTTGTTTAAGTTCCTTGTAGATTCTGGATATTAG
ATCTTTGTTAGATGGGTAGATTGCAAAAATTTTCTCACATTCTGCAGGTTGCCTGTTCACTCTGATGACA
GTTTCTTTTGCTGTGGAGAAGCCCTTTAGTTTAACACTTGGCTAATTTTTGTATTTTTAGTAGAGACATG
GTTTCACCATGTTGGCTAGGCTGGTCTCGAACTTCTGACCTCAAGTGATCCACCTGCCTTGGCCTCCCAA
AGTGCTGGATTACAGGCGTGAGCCACTGCACCTGGCCCCCACACCATTTCTTTAGCAGAAACAACTGCTA
TCCTGGTGATGGTGTTAACACTTGACAACCGTATTTTTGTATGTATACATATTCACAAACAACACATAGT
GTGATTTCATGTATTATAAGAAGTATATAAGGCAACACCATACATGAAATTTGCAACCAGTAAATGAGAA
CCTTACCAGGTAACTTATATGTTGTCCTTGGGGTAGGTTTGGTTTATGCAGAGCTGGTTTTTGCACTTTA
CCGGGAATAATGTGCTTTCCCAGGAATTGCTGGGTTTATGTTTAACTTTTAAAAATACCAAGCTGTTTTC
CAAAGTGGCTAAGCTATTTTATATTCTCATCAGCAACATTTGAGGGTTTCAATTTAACCACATCCTCACC
AATGCTTGATAGCTGTTAGTCTTTTAGATTATAACCATTCTAGTGGGTGTGTACTGATAACGCATTGTGG
TATTAACTTGCATTTCCATAATGACTAAAGTGAGCACCTTTTCATGTGCTTATTAGCTATTCATATGTTT
TCTTTGGTGAAATGTACATTCAAACCTTCTGCGCAGTTTTTAATTGGGTTGTTTATTTTCATATTATGAA
AGGGTAAGAGTTCTTTATATATTCAACACAGTAATTCTACTGCTAGGTACCTAGAAGTAAGAATATATAT
CCACATTTGTATGTTGATATAAAGACATCATTCGAATGTCTATAGTGGCATTACTCATAATAGCCAAAAT
GTAAATGTTTATCAACTGGTAAATGAATAAAAAATAGTATATCCATGCAAATAATACTATATAGCAATAC
AAAGGAATGAAGTACTCATACATGCTACAACATGGATGAAACACTAAAACACACTAAGGGCTGGGCGTGG
TGGCTCATTCCTGTAATCCCAGCACTTTGGGAGGCCAAGGTGGATCACCTGAGGTCAGGAGTTTGAGACT
AGCCTGGTTAACATGGTGAAATCCCATCTCTACAAAAATACAAAAATTAGCTGGGCATGATGGTGGGTGC
CTGTAATCCCAGCTACTGGGGAGGCTGAGGCGGGAGAATCGCTTTAACCTGAGAGGCAGAGGTTGCAGTG
AGCTGAGATTGCGCCATTGCACTCCAGCCTGGGCGACAGAGTGAGACTCCACCTCAAAAAAAAAAAAAAA
ACACTAAGTAAAAGAAGCCAAAAGATTATATATTGTATGAATCCATTTATATAAAGTCAGGAAGGCAAAT
CTATAAAGTAGTGGTTGCCTGGGGCTGTGGGTGGGTATGTGGAAGCTTTCTGGGGTAACTGAAATGTTCT
AAAACTGGATTGTGGTGATGGCTGTACAACTCTAAGAATTTACAAAAATTATTAAAATGTGCACTTATAC
AGAGTGAATTTTATGATCTAAAATTTACATCCCAATAAAGCTGTTTAAAAACAAACACACAGACAAACAG
AAATAATACCAACCATAACTAAATTCATGTAGAAAGTAGAAGAGAATTCTTTTCAACTTTTTCTTTTAGG
CTGGCAAAACCGTGATACCAAAAGCTGACAAAGACATTATTAAAAAAGAAATTTATGGACAAATATTCCT
CATGAACACAAATGCAAAAGTCATTAACAAAATATTAGCAAATTAAGTCTATTAATAAAGAACAAGCTTA
ATACATCATGACAATATGGGGTTTATCTCATATGAATTCAAGGTTGATATAACATTTGAGAAAAAATAGA
GTTCACCACATGTGAACGACAGCCCTCCAAAAATATCTATGCCCTAATAATCCCTGCAACCTGTAATTAT
CTTGCATGTCAAAGGGAAATTAAGGTGCCAAAGGGAAATTGAGGTAGCCAATGTAATTAATCAGCTAACC
TTAAAATATGCAGATTATCATGAATTACCTAGTTGGGTCTCATGTAATCACAGGGACCCAAGTGTGGAAG
AGGGAGGCAGAAGAGTATCAGAGTGATGTGGTATGAGAAGGACTCCACTGGCCATTGGTGGTTATGAAGA
TGGATGGGGGCCATGAGCCAAGGAATGCAGGCAGCCTCTAGAAGTTGTAAAAAATACAAAAACTAATTCT
CCCATAGACCTTCCAGGAAAGAACACAGCCCTGTTAACACCCTGAGTATAGCCCAGAGAACCTCATTTTG
TACTTTTGAATATACAGTAAATTTGTGTTAAGTCACTAGCTCTGTCGTAGTTTGTTACAGCAGCAATAAG
AAATCAATATACCACATTAACGAAATAAAAAATTATATGGGCCAGACGCGGTGGCTCACACCTGTAATCC
AAGCACTTTGGGAGGCCAAGGCGGTGGATCACCTGAGGTAAGGAGTTTGGGACCAGTCTGGCCAACATGG
TGAAACCCTGTCTCTACTAAAAAATACAAAAATTAGCCGGGCATGGTGGTGGGCACTGTAGTCCCAGCTA
CTCAGGAGGCTGAGGCAGGAGAATTGCTTAAACCCAGGAGGCGGAGGTTGCCAGTGAGCCGAGATGGCGC
CATTGCACTCCAGCCTGGGCAACAAGAGCAAAACTCTGTCTCAAAAAGCAAACAAACAAACAAGCAAACA
AAAATTATATAATCACTTTAATGCAGAAAAAGCATTTGACAGAACTCAATACACATTCATGATGAAAACA
TTTTTTTTTTTTTTGAGACGGAGTCTCGCTCTGTTGCCCAGGCTGGAGTGCAGTGGCGCGATCTCGGCTC
ACTGCAAGCTCCGCCTCCCGGGTTCACGCCATTCTCCTGCCTCAGCCTCCCGAGTAGCTGGGACTACAGG
CGCCCGCTACCACGCCCGGCTAATTTGTTGTATTTTTAGTAGAGACGGGGTTTCACCGTGTTAGCCAGGA
TGGTCTCGATCTCCTGACCTCGTGATCCGCCCGCCTCGGCCTCCCAAAGTGCTGGGATTACAGGCGTGAG
CCACCGCGCCCGGCCGAAAACATTTTTTTAAACCAGAAATAGAAATAGCAGACTTTAAGATAAAAAGCAT
GATCAAATAAAAAAGGAGAGATTTAATGATGATAAAGTGTCCATCTGTCAGAAGATATAAAATTCTAAAT
TATATGCCACTAATAATATAGCCTCAGGGAAAAAAACTGGCAGAAATAGAAGGAAAAGGAAATTATAACT
ACAGTGGATGGGTTTTTTAAAAAATTATACTTTAAGTTCTAGGGTAGATGTGCACAACGTGCAGATCTGC
CACATAGGTATACATGTGCCATGTTGGTTTGCTGCACCCATCAACTCATCATTTACATTAGGTATTTCTC
CTAATGCTATCCCTCCCCCAGTCCCCCATTCCCCTACAGGCCCCAGTGTGTGATGTTCCCCAGCCTGTGT
CCAAGTGTTCCCTCTGTTCAACTCCCACCCAAGAGTGAGAACATGTAATGTTTGGTTCTCTGTCCTTGTG
ATAGTTTGCTGAGAATGATGGTTTCCAGCTTCATCCATGTCCCTGCAAAGGACATGAACACATCCTTTTT
TATGGCTGCATAGTATTCCATGGTGTACATGTCCCACATTTTCTTAATCCAGTCTATCATTGATGGACAT
TGGGTTGGTTCCAAGTCTTTGTTATTGTGAATAGTGCCACAATAAACATATGTGTGCATGAGTCTTTATA
GTAGCATGATTTATAATCCTTTGGGTATATACCCAATAATGGGATTGCTGGGTCAAATGGTATTTCTAGT
TCTAGACCCTTGAGGAATCACCACACTGTCTTCCACAATGTTTGAACTAATTTACACTCCCACCAACAAT
GTAAAGGCACTTCTATTTCTCCACATCCTCTCCAGCATCTGTTGTTTCCTGACTTTTTAATGATTGCCAT
TCTAACTGGTGTGAGATGGTATCTCATTGTGGTTTTGATTTGCATTTCTCTGATGACCAGTGATGATGAG
CATTTATTCATGTGTCTTTTGGCTGCATAAATGTCTTCTTTTGAGAAGTGTCTGTTCATATCCTTTGCCC
ACTTTTTGATGGGGTTGTTTGTTTTTTTCTTGTAAATTTGTGTAAGTTCTTTGTAGATTCTGGATATTAG
CCCTTTGTCAGATGAGTAGGTTGCAAAATTTTTCTCCCATTCTATAGGTTGCCTGTTGACTCTGATGGTA
GTTTCTTTTGCTGTGCAGAAGCTCTTTAGTTTAATTAGATCCCATTTTTCTATTTTGGCTTTTGTTGCCA
TTGTTTTTGGTGTTTTAGACATGAGGCCTTTGCCCATGCCTATGTCCTGAATGGTATTGCCTAAGTTTTC
TTCTAGGGTTTTTATGGTTTTAGGTCTAACATTTAAGTCTTTAATCCATCTTGAATTAATTTTTGTCTAA
GGTGTAAGGAAGGGATCCAGTTTCAGCTTTCTACATATGGCTAGCCAGTTTTCCCAGCACCATTTATTAA
ATAGGGAATCCTTTCCCCATTTCTTGTTTTTTTCAGGTTTGTCAAAGATCAGATAGTTGTAGATGTGTGG
TATTATTTCTGAGGGCTCTGTTCTGTTCCATTGGTCTATATATCTGTTTTGGTACCAGTACCATGCTGTT
TTGGTTACTGTAGCCTTGTAGTATAGTTTGAAGTCAGGTAGAGTGATACCTCCAGCTTTGTTCTTTTTGC
TTAGGATTGTCTTGGCTATGAGGGCTCTTTTTTGGTTCCATATGAACTTTAGAGTATTTTTTTCCAATTC
TGTGAAGAAAGTCATTGGTAGCTTGATGGGGATGGCATTGAATCTATAAATTACCTTGGGCAGTATGGTC
ATTTTCACAATATTGATTCCTCCTATCCATGAGTATGGAATGTTCTTCCATTTGTTTGTGTCCTCTTTTA
TTTCGTTGAGCAGAGGTTTGTAGTTCTCCTTGAAGAGGTCCTTCACATCCCTTGTAAGTTGGATTCCTAG
GTATCTTATTCTCTTTGTAGCAATTGTGAATGGGAGTTCACTCATGATTTGGCTCTCTGTCTGTTGTTGG
TGTATAGGAATGCTTGTGATTTTTGCACATTGATTTTGTATCCTGAGACTTTGTTGAAGTTGCTTATCAG
CTTAAGGAGTTTTTGGGTTGAGATGATGGGGTTTTCTAAATATGCAATCATGTCATCTGCAAACAGGGAC
AATTTGACTTCCTCTTTTCCTAATTGAATACCCTTTATTTCTTTCTCTTGCCTGATTGCCCTGGCCAGAA
CTTCCAACACCATATTGAATAGGAGTGGTGAGAGAGGGCATCCTTGTCTTGTGCCAGTTTGCAAAGGGAA
TGCCTCCAATTTTTGCCCATTCAGTATGGTATCGGCTGTGGGTTTCTCATAAATAGCTCTTACTATTTTG
AGATATGTTCCATCAATACCTAATTTATTGAGAGTTTTTAGCATGAACTGGTGTTGAAATTTGTCCAAGG
CCTTTTCTGCATCTATTGAGATAATCATGTGGTTTTTGTCGTTGGTTCTGTTTATGTGATGGATTACATT
TGTTGATTTGCATATGTTGAACCAGTCTTGAATCCCAGGGATGAAGCCAACTTGATCATGGCACATAAGC
TTTTTGATGTGCTGCTAGATTCGGTTTGCCAGTATTTTATTGAGGATTTTTGCACCAGTGTTTATCAGGG
ATATTGGTTTAAAATTCCCTCTTTCTTATTATGTCTCTGCCAGGCTTTGGTATCAGGATGATGCTGGCCT
CATAAAATGAGTTAGGGAGGATCCCCTCTTTTTCTATTGATTGGAATAGTTTCAGAAGAAATGGTACCAG
CTCCTCTTTGTACCTCTGGTTGAATTCGGCTGTGAATCCATCTGGTCCTGGACTTTTTTTGGTTGGTAGG
CTATTAATTTTTGCCTCAGTTCCAGAGCCTGTTATTGGTCTATTCAGAGATTCAACTTCTTCCTGGTTTA
GTCTTGGGAGGGTGTATGTGTCCAGGAATTTATCCATTTCTTCTAGATTTTCTAGGGTTTTTTTTTTTTT
TTTTTGCATGGAGGTGTTTATAGTATTCTCTCACGGTAGTTTGTATTTCTGTGGGATTGGTGGTGATATC
CCCTTTATCATTTTTAATTGCGTCTATTTGATTCCTCTCTTCTTCTTTATTAGTCTTGCTAGTGGTCTAT
CAATTTTGTTGACCTTTTCAAAAAACCAGCTCCTGGATTCATTGATTTTTTGAAGGGATTTTTGTGTCCC
TATCTCCTTCAGTTTTGCTCTGATCTTAGTTATTTCTTGCCTTCTGCTAGCTTTTGAATGTGTTTGCTCT
TGCTTCTCTAGTTCTTTTAATTGTGATGTTAGGGTGTCAATTTTAGATCTTTCCTGCCTTCTCTTGTGGG
CATTTAGTGCTATAAATTTCCCTCTACACACTGCTTTGAATGTGTCCCAGAGATTCTGGTATGTTGTGTC
TTTGTTCTCATTGGTTTCAAAGAACATCTTTATTTCTGCCTTCATTTTGTCATTTACCCACTAGTCATTC
AGGAGCAAGTTGTTCAGTTTCCATGTAGTTGTTTCCATGTAGTTGAGTGAGTTCCTTAATCCTGAGTTCT
AATTTGATTGCATTGTAGTCTGAGAGATAGTTTGTTGTGATTTCTGTTCTCTTACATTTGCTGAGGAGTG
CTTTACTTCCAATTATGTGGTCAATTTTAGAATAAGTGCGATGTGGTGCTGAGAAGAATGTATATTCTGT
TGATTTGGGGTGGAGAGTTCTGTAGATGTCTATTAGGTCTGCTTGGTGCAGAGCTGAGTTCAAGTCCTGG
ATATCCTTGTTAACCTTCTATCTCGTTGATCTGTCCAATATTGACAGAAGGTTGTTAAAGTCTCCCATTA
TTATTGTGTGGGAGTCTAAGTCTCTTTGTAGGTCTCTAAGGACTTGCTTTATGAATCTGGGTGCTCCTGT
ATTGGGTGCATATATATTTAGGATAGTTAATTAGCTCTTCTTGTTGAATTGATCCCTTTACCATTATGTA
ATGGCCTTCTTTGTCTCTTTAGATCTTTGTTGCCTTAAAGTCTGTCTTATCAGAGACTAGGATTGCAACT
CCTGCTTTTTTTTGCTTTCTATTTGCTTAGTAGATCTTCCTCCATCCCTTTATTTTGAGCCTATGTGTGT
CTCTGCATGTGAGATGGATCTCCTGAATACAGCACACTGATGGGTCTTGACTATCTAATTTTCCAGTCTG
TGACTTTTAATTGGGGCATTTAGCCCATTTACACTTAAGGTTAATATTGTTATGTGTGAATTTGATCTTG
TCATTATAATGTTAGCTGGTAATTTTGCCCGTTAATTGATGCAGTTTCTTTATAGCATCGATGGTCTTTA
CAATTTGGCATGTTTTTGCAGTGGCTGGTACCAGTTGTTCCTTTCCATGTTTAGTGCTTCCTTCAGGAGC
TCTTGTAAGGCAGGCCTGGTGGTGACAAAATCTCTCAGCATTTGCTTGTCTGTAAAGGATTTTATTTCTC
CTTCACTTATGAAGCTTAGTTTGGCTGGAAATGAAATTCTGGGTTGGAAATTGTTTTCTTTAAGAATGTT
GAATATTGGCCCCACTCTCTTCTGGCTTGTAGGGTTTTTGCTGAGAGATCTGCTGTTAGTCTGATGGGCT
TCCCTTTGTGGTAACCCAACTTTTCTCTCTGGCTGCCTTTAACATTTTTTCCTTCATTTCAACCTTGATG
AATCTGACAATTATTGGTCTTGGGGTTGCTCTTCTCGAGGAGTATCTCTGTGGTGTTCTCTGTATTTCCT
GAATTTGAATGTTGGCCTGCCTTGCTAGGTTGGGGAAGTTCTCCTGTATAATATCCTGAAGAGTGTTTTC
GAACTTGGTTCCATTCTCCTCGTCACTTTCAGGTACACCAATCAAACATAGATTTGGTCTTTTCACATAG
TCCCATATTTCTTGTAGGCTTTGTTTGTTTCTTTTTACTCTTTTTTCTCTAATCTTATCTTCTTGCTTTA
TTTCATTAATTTGATCTTCAATCACTGATATCCTTTCTTCCACTTGATCGAATTGGCTATTGAAGGTTGT
GCATGCATCACGAAGTTCTCGTGCCATGGTTTTCAGCTCCATCAGGTCATTTAATGTCTTCTCTATGCTG
TTTATTCTAGTTAGCCATTCGTCTAACCTTTTTTCAAGGTTTTTAGCTTCCTTGCGATGGGTTAGAACAT
GCTTCGTTCGCTCAGAGAAGTCTGTTATGACTGACCTTCTGAAGCCTACTTCTGTCAACTCGTCAAAGTC
ATTCTCTGTCCAGTTTTTTTCCGTTGCTGGCGAGGAGCTCTGATCCTTTGGAGGAGAAGAGGTGCTCTAG
TTTTTAGAATTTTCAGCTTTTCTGCTCTGGTTTCTCCCCATCTTTGTGGTTTTATCTACCTTTGGTCTTT
GATGTTGGTGACCTACATATGGGGTTTTGGTGTGGATGTCCTTTTTGTTGATGTTGATGTTATTCCTTCC
TGTTTATTAGTTTTCCTTCTAACAGTCAGGTCTCTCAGCTGCAGGTCTGTTGGAGTTTGCTGGAGGTCCA
CTCCAGACGCTGTTTGCCTGGGTATCACCAGCGGAGGCTGCAGAACAGCAAATATTGCTGCCTGATCCTT
CCCCCGGAAGCTTCATCCCAGAGGGGCACCTGCCTGTATGAGGTGTCTGTCGGCCCCTACTGGGAGGTGT
CTCTCTGTTAGGCTACACAGGGGTCAGGAACCCACTTGAGGAGGCAGTCTGTCTGTTCTCAGAGCTCAAA
CACCATGCTGGGAGAACCACTGCTCTCTTTGGAGCTCACCTGGAAATGCAGAAATCACCTGTCTTCTCCA
TCGATCACGCTGGGAGCTACAGACTGGAGCTGTTCCTATTTGGCCATCTTGGAATGGACCCCCCTATAAA
AAAAAAAAAAAGGAAATGGATGTTTTTAATACATCTCTCTCAGTAAACCTAAGTAAAGATGTAAGAGATT
TGAGAAACAAGACTAACAAACTTGAATTAATGGACATATGAAGAAGATTCCACCCCAAAATGTCAGAATA
TGTCCCTCTTCAAATACAAGTAAAACTAATATTGGCTGAGCATGGTGATGACGTCTGTAATTCCAGCACT
TTGGGAGGCCAATGTGGGCGATCATTTGAGGCCAGGAGTTCGAGACCAGCCTGACCAACATGGCAAAACC
CCGTCTCTACTAAAAATACAGAAATCAGCTGGGTGTGGTGGCACGTACCTGTAATCTCAGCTACTCAGGA
GGCTGAGGCAGGAGAATCACTTGAGGGGGATAGAGGTTGCAGTGAGCTGAGACAGTGCGGCTGCACTCCA
GGCTGGGTGACAGAACAAGACTCTATCTCAAAAATAAATAAATAAATAAATGAAAATAAAACTAATATTA
AACATATGTTGAGACACAAATGCTCAACAAATTCAAATGAATAAAATAATATAATCCTTGACTGAGTAGA
ATTAA

An exemplary DNA sequence of human Anapc13 is provided below.

gi|224589815: c134206112-134195297 Homo sapiens
chromosome 3, GRCh37.p13 Primary Assembly

(SEQ ID NO: 304)

CTAAAAATGGTACTGCATGCCAAATAAGAAAATATATACACTTTATCATT
AAGCTACAGCTAACAGAAATCTAATCAACGATCCTTATGATTTCCTCGTA
TCTAAAAAATAAATGCAAGCACTTGAGGAACTGTATAAATACAGGGCATC
TACCCTACAATACTGCTTTATTAAAATAATAAATTCTTTTAATAAGTTTA
ACCAGACATAAGGCTCAGGGAAAACTACTCTGTGCATATCAGCGTACGGT
GCTGAAACTGTAAAAGACGACCTGTCTCTGACATCATACTCATCTTTACT
CTCCTGATGTCGTAAGGGACGTAAGAAAAACCACATTATAAGCCTAAATG
TACTGTCTTCAAGTATCTCCATGATTAAAGTGTTCCTAAGAAACCGTCCT
TATTTCTTTCTATCCCTCATTTGTGTGCATGGAGCCGTTAGTTCAGGGTT
CTCCAATTTCAGTCCACGATTTATGCCACATTTCCGTACCTCTTGTACTA
ACATAACCGTTTAAACCAACTCACTATTTTAAAAGCTGCCAACCTTAACA
TGCCACACTAAATGAGCGGGTCCCCTTGTGCCGTGAATGAAGTAGTCCCA
ACAGCGCTAGCTAGACAGCAGCCCTGGCAGGGAGGCCGCACTGGGGTCTC
TCCGCTGAAAAGGAAGGTGGGTCCGCCGGGGAAGGTGAGTACTGGGCGCA
GCTCGCTCGCCCACCTGGCCACTCTCCGAAGCGCCCCCAGCCACCTGGAC
TCCGAGGAAGGGCTGACCACCTGGCGGGGCCGAACCCCACGCCGGGCTGC
GCCACGCCAGACTCCGCCAGTGTGCAGGGCATAGCGGGCCAGGCCGAGGG
ACTGGGCTTCGGGGCGGACCCAAGCCGGCGGGGACGCGCCTGGTGGTTCG
CAGGCCGTTGCGCAAACCGGCCGCAAGCCATGAGGCCGCGAAGCCAGAAT
CACCAAGGCAGCCTGGGCCGCCTCCAGACCAGGACACGGCAGCCCCTTGC
CCCTCCAACTTGCGCACCGGCGGTTACAGGGTTGCCGCAAGCACGCCCCT
GAGCCTTCCCGCCGTTACCTCGGGCCGGCGCTGCGCACGCGCGCCACCGC
TAAGCTTACGTCCAGCCTCTGTAGTCGGCGCGTTTGTTCCGCCCACTGCC
CCCACACTTTTAATAATTGAAACAATGCCTTCTCCAGACTTCCCCTGCAC
GCGAGCAAATGAACTCACCCACATCCGGGAATCCGAGGAAAGCGAGTGCA
GACACGTGATGCGGGGGAGGGGGGGGGGGCGTGGCAGGAGCAAGCGTCTG
CCGCGGTGGCCGGGTGCCGGTAAGGGTTTCCAGCGCCCCCGGCCTAGGTT
TTGGAGGCGCGGGAATGCGTTCGTTGCTCAGTGTCGGACTTCCCCCTATT
CCCATCGGCCGAGGCTGTCACTTTACGCTCATAACCGTTTTTCTTTACTG
CACTCGTGTCGGGAGGAAAGGGACTTGCGTGGCACCCCCAGACCTCCCCG
TCTCCGCTTCCACGTTTGGTACATCCTGCCTGAGGCAGGAAGCCGCAGCT
GAGGGACGGCCTGTCGTACGGTGCGGATGGTGGTGGCCTGCGAGGCTCAT
TTCTAGCAAGGAACAAGGCTTTCCCGCTTTGATTTTATAAATATTATGTT
TACAAAGCTGTAATATATAGAAATTGATAAGACGTGTCCCTGTCCCTGGA
AACGCAGGCACCGCGTGTTTGGAAAGACATTCATCTGGGCTGTTTGACAG
ACTCCCCAGTTGGTGCCATGCTCTGTGCTTAGGGAACTGTGAGACCCCTG
GAGGGGTGGGTACCGGGACCGCACTCAGCCTGGGGTTTGGAGGCGGCCTC
CTATAGGAAGCGACCTGGGACCTAAGATTTTTAGACTGACTGTGGGTTCA
CTGGAATAAAAAGGAAGAAACAAAGAGCATTGCAGGCATCGGGACTGTCA
CGTGAGTAAGAAAAGTTTGTTCAGGAAACAGTGGTCGTACTGGGTGACGA
GTACTTGGGACGGTGGCTTTAATACACAAGATAATCAGTGTGCCCTGGGC
ATTGATTATCTTGATGCTTCCCATCCTTCCAGCCCCTTATGAGGGAAGGC
ACCTTCGCGCCTAATCCCGGAAATGGGATGTTGACCTTCAAGAGCTGACG
CCTGCGTTTATCTTGAAAGGAAGAGTGGCATTAGTGATGTGGAAAAGGGG
GTTTTAACTGTCAAGGCTGGAAAATGTGACAGGGTTGGAGGTGTTTGGAG
AAGGGACAGGTGAAAAAGATTGAGACCAAATGTGGAAAGATCTACTAAGG
AATCATGGGTACAAAATGTACACGTAACAGTTGGAGGCAATAATACCTGA
GCAATTGACCACAAAGCTGACCTAGCTTGTTTAGTAGATTAAGTTAGGAC
AAGAAGCAAGAGTTCAAGGATAGAAGGCCTACTGAAGCTCGAGTGATTTG
AGAAAACTTTACAAAGGTGGAAAATCTACGTGGGCCTCCGAAAGTCAGGT
TTGGATCAGCTGAGGAAAGTGAGAAGGGTATCCCAGGAGACAGAGCAGCA
AGGACAAAGGAGTAGCAGCAGGAAAGTGGGGAGCACTTGTAAGACAGGGA
CTTCAAAAGTATAACCAAAACTTCGTCTTGCTTTGAGTTATCCACTGGGT
AGTACTCGGTTAATTATTGAGTACTGGATTAATTAAGCCCTCACCTGGCT
CACCGGTGACAGAAGTAGCAGGGCGCATTTTGCTTAACTTTTGCCGAGAT
CCTTGTCATGGGGGTGTAAACCCTTTCCCACACCAATCTGTGTATAATTT
TTGCCAAGGTGTTAGGGATATCTTGCAGTTATCCTATGGGGGACAGCTGT
GAGCCTTTTTGGTTTTTTTTGTTTTTTTTTTTCCGAGACGGAATCTTGCT
CTGTCGCCCAGGCTGGAGTGCAGTGGCGCGTGGCGCTATCTCGGCTCACT
GCAAGCTCCGCCTCCCAGGTTCACGCCATTCTCCTGCCTCAGCCTCCCGA
GTAGCTGGGACTACAGGCGCCTGCCACCATGCTCGGCTAATTTTTTGTAT
TTTTAGTAGAGACGAGGTTTCACCCTGTTAGCCAGGATGGTGTTGATCTC
CTGACCTCGTGATCCGCCCACCTCGGTCTCCCAAAGTGCTGGATTACAGG
CGCGAGCCACCGCGCCCGGCCCTGTGAGCCATTTTTACAAAAACTTCCAC
CTGCCTTGGCCCAAACCTCAGCGTTCTTAGGCACAGGAGCTCATGAACAT
ACTGAGATAACACTAAACTGGACTATGCGAAGGGACCAACTACAGACCTG
ACACAAATCCTCAAGGCAAGACGATGTCTGAAGTTGAGCTTGTAAAGGAA
ATGGCTTCCTTCCTTAAAATATCACCCAGGCTGAGCTCTGAAAAAAGCAC
CTCCAGATGGTGGGTTCCACCCCGCTCCACCACCCCAAAATTGCTTATTG
TGTGAGTCTCCATAGTCTCAGAAACTGAGAGTACCTTTGATGCCAGTGGC
TTCCGGGCTTTTTGGATTGTCACTTGGTAGCAGGGAGCACCCATAGGATG
ATGCCTATGATGAGTCAGGGCCCAACCTTGGAAACAGGGAAATCCAGAAG
TATCCTACAAGCTAGCCAGCCTTTTGGATTAGATTATAGCAGTTTCAGAG
AAAAATGGGGAATCAGCCATGGTCAAGGAGCTAAATAAGCATCGTGACCA
TTTGGGCCAGAAATTGAAGGGAGTGTAGCGTTAAGAATTAGAGGAAGGAA
CCTCCGAGGAAGGAGCTACCCCAAAGGGAAGCAGAAACTGGAGAAACTTT
AGCAGTATGAGGTTCTACCACTGGTGTTCACCTAGAAGTGCTTTATTCAG
ATCTCAGCCTGGGTACTATTTTAGGGAAGGAAGCCGTTTCCTTTACAGTA
TGTTCGTGTGACTCCTGTGCCTAAGAATGCTGAGGTTTGAGCCAGGGGCT
AGAAAGGAAACTTAGCTGGCCATTATGAATCAGGGACTCATCAAAGGCAT
CACAAACAGGAAAAGGTGAATGTTCAAGTTAGAAGAGAAGAGCTACATTG
AGATGCAACATATTAATATTTGAGGGCCTCGTTTAACGTGTTGCTCTCCT
GTCACACTCTATGACACTGGTCTGTCAGCTGGGCTATGCCTGTCCAGTAT
ACACTATAAAATATGTATCAGTTGGGGTTGGAGAGAGGAACTGTGATCAA
AGTAGTCACCCGGCCCATATGAGAAAATGAACCTTTTCCCAAAAGGTGGC
CCCAGACTAAAAAGCCTGGAGAATCCTAATAGTGGATTATACCATCTCTT
CAGTCTTCGTGTTTAAGAAATAACCTCTTTGGCTTACAGATTTGACAAGA
TCAAAGCTGCAGGAAAATGGACAGTGAGGTTCAGAGAGATGGAAGGATCT
TGGATTTGATTGATGATGCTTGGCGAGAAGACAAGCTGCCTTATGAGGAT
GTCGCAATACCACTGGTAGGTTATGATTTGAGCTGAGGATGGGTTAAAGG
TATTGATCTGGCAGTAACTAGTGGAGGAGGGATATCAATGGAAGATAAAC
TAGTGTGGCAAAGAAATCAAAGCTGTGGAATTCTCTAACTCTAGAAGCTT
CTGGAGAGCATCCAGTTGTATGTTTTATTTGAAGAAAAATGTGGTTCAGG
GAGCTAACATGGCCAGGTCAGTTAGGAGTCATGCCAGGACTAGAGCCCCG
ATTTTCTGAGCCATGTTCTGTCCACCACACGAAAACCAGAGAAACCAAAT
AATGAGAGTTTAATGCGCAAGATGGGGAAGAACAAGAAGCAGCAGCATAT
GAGCTCTGTTTCTTACTTCCCTGCCTGTGGCTCATCAGTCTTTCAGCATT
TTTAGAAACTTACATTCTTGGAGCATACACCTATCTTAGGTTCCCTGGAA
AATATTCAGAAGATACCTGGGTATTCTTTTGCGTTTAGGCCAGTGGCCTT
CTGGAATACTCTTTTCTTCCCCATCTTTGGAAAACTCTGCTGCAGGGAGT
TTGTTAAACCTCATGTAATAAGTGATCCAAAAGTTTTGCTTATATAAATG
TTTGTTTAAAATCCTATTTATGTCATAGAGGCCTAAAAAAGGGAAAACAG
GACTGTTTTTCTTTAAAGCATGCCAGCTGCCCTGAGAAGAGTAGTTAGTA
TTGAATAAAATATAATTTATAGTTAGAATATCTGCTGTTGCTGTTTGCAA
AGAAATAAAATAGTTCATCTGTATTTTCATTTACGAATAAGAGATAGTAA
TATGTTCAATGAATAATATACATATCAAATAAGTCATAGCTTAGTTCAAG
CTTAGGAAAATAGTCAAAAGAGAGAGCATAATAAGAATTATAGATGTCAT
TTATTGAATACCAGTTTTATGCCATGGACTGAGGTTGGAAATATACATTA
TCTCTAATTCTTAAACAACCCTGTGAGGGAGGTGCTGTTATTCCCATTTT
ATAGGAAAAGAATAGGATCAGAAAGGGCAACTTGCCCAAAGTCTGGTAGC
AGGTAAGTAGGGCTTCATATAACATATACCTAGGTCTGTGAAGTTCCAAA
TCCTGTCTTGTGAACTTCTGTTCATTCATTCATTTGACAAGCATTTTTTG
AAGCCTACTGTGTGCCATAAGACTATGTGAACATAAGATACAGTTCCTCC
TCCTGAGGGGTCTAGAAAGGCAACACTTATAGATAAGTAATTGTGATAAT
AAATATTCTAGGTGGGGGCATAAACAGCCACATCATATACGAAGTGGCTG
ATTGCCTGGTGGGCATCAGAAAGGCTTCTGAGAGAATGTCATGTTTAAGC
TGGGTCATAAGGAAGGACCAGAAGTTTAGTGGAGGTAGACAAGGGTATTG
CAGCAGAGGGGACAGATGTGTAAAGGCCCAGAGTTTTGAAAGGCTTTGGG
ACATGGTGAGTCATTCACTGTGGATGAAGAATACATTTGTCACATAAGTA
GGCTGAAAAGTTGGGCTTGGATCAGATTGAGAGTGGTATTTGATATCACA
CTTAGACCTCATCCTCAGGAGATCCAGGAATGAATGGTCTTAAAAGCAAG
ACTGCTATGCTCAGATTTGGCTTTTGGAAAATCAGTTTAGCCAGCATTGT
TAGAGATGGTGTGACATGGAGATAGGGGCAAGTGAGACCAATTGGCAAGC
TGTTGTGGTCTAGGCAAGGGAGGAGGGCCTAAACAAAGTCAAGAAAATAA
AGGAGACGTCCATTTCTGGAGAAGTTTATGGCTCAGATTGGAGAACATTT
GGGTGGCAGGGGTGGAAGGAGGTTTCCCTGCTGACTTCCAGGCGAAGTTG
GCTTGGCCCTGCTGCTAATCCACACCTTTCGCTTGTCTTGCGATTCTAAG
GCAATGTGTATGCCAGTTAGGTTTTGCTGCATAACAAGTCACCCCAAAGC
TTAGTGACTTAAAAGAGCAACTGTTTATTTCATGATTTTGTAGCTCAGCT
GGGAGGGACTTCTAGTCTGCACCAGCTCAGCTGATCCCTGCCAGGCTTGC
CTGTGTTGTGCAGTCAGCCAACAGACTCTGGAGGCTGGGTAATCTAGGAC
TGTCTCACATGTCTGGCAGTTAACAAGCTGTCTGCTGGGTGATGGGGTGA
CTATGCCATGGTCTCTGGTATCCAGCAGGCTAGCCCAGACTTCTCAATCT
CAGGGTTCCAAGAGCAACCTGAGAGGAAACGGCAGAGCTTCTTGGAGCTT
AGGCCAAGAACTGGCCCACTGGCATGACAGCTTCATTCTGTTGGCCAAAA
CAACTCACAGGGCCAGTCCAAATCCAAGATTGGGGAAATCAGTTTCACCT
TTTGATAAGAGTAGCTGCAAAGAACTAAGGGCATTTTACAGTACACCATA
ATGGGTGGCTGGGTTTATGATACTTTTGTTTTTGTTTTCCAGGCATATAT
GCCATTTTATGTGTTGGCATGTATTGCAAGGGACAGTAGATACCAAATGA
AAGACACATTATTTTAGAAAGCATACCACAGATAGGATGAAATTAAGCAA
CGTTGTCTGCTTCTGAATTAAAGAATTCATTAACCAAGGACCTATTTATT
CTTTGTCATCTATAAGTAATTATTAGGACTGAACTTTCTTTTTAAGTTTA
AGATAATATATTATTGCGTTACAATAAATGATAGCTCTTCTCAATCTGTT
CAAGTATGTTTGAATCTGTCTTGTAGAACAAGATGTATGTGATTCAAATA
TAGATAAGTAAATATCATTTAATATATATTGAGCATAATTTTAGAGGGGA
AAACATATTCACATACTACAAATTAGAATCCAGTGGAAAATTTTAAGTCT
GGATATCGCATAACAGTTTGCTAACCATAAAACAAAACACAGATAATTGA
AAAGAAATATATTGGTATCTGTGAACTCTGGTACAAAGCAGAGTTTCAGT
AGTAATACATTTAAACTTAAAATACAAGTAAATGATCCCAGATCCTACAG
TAAGTGTGGCCCATGACTTACCATTTTAAGAAAATTGCTGGTAGGGTGTG
AATTGGTCATTTGTTGTGTTTTTTGTTGCTCTGCTCCACCACTAGAGGGA
GGTCTTATCTATTAATTACATTTTCAGTGTTTCCTGCCCTACCTCCATTT
TTATTATAATATAGACATGTTGGGCCAGGCGCGGTGGCTCGCGCCTGTAA
TCCCAGCACTTTGGGAGGCTGAGGCGGGTGGATCACCTGAGGTTGGGAGT
TCGAGACCAGCCTGACCAACATAGAGAAACCCCATCTCTACTAAAAAAAA
ATAAAAAAATACAAAATTAGCCGGGCATGGTGGTGCATGCCTGTAATCCC
AGCTACTCGGGAGGCTGAGGCGGGAGAATCGCTTGAACCCGGGAGGCAGA
GGTTGTGGTGAGCCGAGATCACGCCATTGCTCTCTAACCTGGGCAACAAA
AGCAAAACTCCATCTCAAAAAAAAAAGACATGTTGGGTCCAAGATAATTT
TTTGGATACATTTCTTGTGTCTAAGGAATATGCCAGCAAACTAGTATTCT
TTTAAAGCACGCTCCCGCTAAAGAAGATTTAGGATTCACTCATCTCAGTG
TTCCTAGCCGCTAATAATAATAGCAAATATTCCTTGAGCACTGGATACCC
CACAGAGTGCTCAGCACTTGATCTGAATTACCTCACCTAATCCTTACAGT
AGTCCTGTGAGGTGATGGCTCTTTTGTTCTCCCATTTGCCAAGGAAGAAA
CAAAGGCCCCAAGGGTTTGCATACCTTGCCCAAGTAACCCAGCAAATGGG
AGAGTTGTGGTCTTGGATCCTAGTTGGCTTCCAAAAGCCTCTGCCCTTAA
CCTTTCTGCCACAGTGCCTCCTTCACAAATGTCAGAGTCAAAATGCCCAT
CATTCCCTACTGAAGAAGGCCCGGGCCATGTTTGAATGATACTTCTGATA
GTTTCCTCACGTGTATGATTTTAGCTTTTTTAGAATACACATATCAATTA
TGTTGCCTGTTCCTAGGAACTCACAAGCTTTAATTCTAAGTGTTCTTGAG
TTCCAAGAAGATGCAGAATATATTACTGTTCTGAATTTCTATTGTACCTT
TTCAGAATGAGCTTCCTGAACCTGAACAAGACAATGGTGGCACCACAGAA
TCTGTCAAAGAACAAGAAATGAAGTGGACAGACTTAGCCTTACAGTACCT
CCATGAGAATGTTCCCCCCATTGGAAACTGACGCTTGGCTCCTTTCTTGT
GGATGGATTTTCTCAAAGTACACAGATAAAGCATGGTTTGTTTCAGTCTC
CAAATTCAAACCTTTGAGTAATAAATCAGCACTCAAAAATGTACACCCAT
TTAGTTTGTGGTAGCAAAGTGCAATGCGAAATTGAATGAGAAACTGAGAT
TTCTCAGTAATGGTGAATATTTCGCTCTTTAAACCTAAAACTCTTCATTG
AGTAGCTTATATTTGAACATGATTGGTTAAACATTTGCCTCTACCTCTGA
TTTTGCTTTGCTGTCAAAGTTTAACACCTTCCAACTACTTATGTGTGTCC
TGTAACACAGGTGATTGAACGTATGAGAGGGAAAGGCAAAGAAAAAGGAA
GCCAGACACTAGGAGAATTATTAACTTCTCATACTTCCCCACATTGAGAA
GCATTCGGAGTGTATTTAGCCTGTAGATGTTGTGATATGCAAATATCCCA
TTCCCTGGTTACTGGCATTCCTAAGATTCTTCATGGTATTTTCAAACTTT
GGATAAATTTACAGATTAGAAAGATATCTGACAGTTAATCTCTGTTCTCC
TTACAAATTCCTTTTGTGCTGCTGGAAAGGATCTTTGGCTAGGTGGATGA
CTAGTTTTATTCAAAGCCTTTTCTCAAAGCCCTTTCAGTTACAACCACCC
CACTATGGAATCAGTATTTAGTTATACATTTGTATAAGAACCTGTATTTT
GAAAAACACATTCATGTATATTTATTCCTGGAATTATTTGCCTGTTAAAC
AGTGTCTTTCATGTTCTCTCCCCAGATTGTAAACTCTGTAAGAAGCTGCT
TGTATCTGTATCCCTTGTTGAAACTCTGAAAACACTGAATAACTAAAATC
TCTTCTCATCCTTTTTATGCTGGGTCTTTTTGTTTACTTAGCTTTTAGGA
GGAAGAGGTACATGAAAAAACAAATTAGCTTATTTTAGGTTTTCAATCAC
AAACATGATTTTAGCAGCTATCCTATAATCCAGATACATCCAGGCTGATT
TCTTCTCTTTGCATCAGTAGTTCTGCCTAGAAATCTTTTATCTTCAGAAT
AAAGACCCCAAGGCACCAGTCCCTTCAGTAAGCAAAGAAAGACAACCTAA
TAGAAAAGAGACTTACAATATTAAAAAAACTTATACAAGAGAAAAAGCTT
TTCAAAAAATTAAAATACCTAGAACGCCACAGTTCTTTCACATTCTTGTT
AAATGAGGATTTTACAGCTTACTTGTGGTATGTAATCTCTGAGACACTAA
CATCTAAGAACTTGACAAATGTTCATGTGAATCCTGCCTAGCCTTTCCCT
GGAATATTAGTAGACCAGTAAAATATGAAAGCTATTCTTGATTTCTGCTG
AACCAGAATATGATATCTGTAAGACAGCTGTTGGTTTGTGTCTATGACAC
TCATCCGAGAACTGCCGCCACCCCCAGGAATAGCCTCTGGGCAAGTTCGT
AACTTGTGTTATGGTGCCAGAACTGTAGCTCATTGTATCAGGAATTAGGT
GACCTAATTTGGGCTAGTCAGATTTTGTCAACCAGGGCTTCGAAATCTCC
GTCTAGGACTGGCTTGAGGATGATGGGCACAAGAAATGCTAAGTTGCTGT
TACAGGGTAGCTTTCTTCAATTGTCTGTACCAAAACACCCAAAAAGGAAA
GTATCTGGAGAGGGAGGCCAAGAGGCTATGTGGCCTATTGGTTCCTGACA
GCTTTCCAGTTTAGAGCCTGCATATTGCTTTTCTCTGTGGGAGGCACCAT
GCCTGGCACTGGGAGTATAGCAATGAACAAAGTAAGTGTGGCCTCTCCCT
TCATGGAGCCTTTAGTTGGGGAGACAGGCATTAAACCATTAGCCATACAA
ATACATGATTTCAATTTGGGGCGTATGCTAGGAAGGAAAAATAAAAGATG
TGACTTAGGTTGGGTTCCCAGAAGCCAAGCCTGAGGCAGAGATTTCTTGT
GCAAGTGATTTATTGAGGTGCTTTCAGAAGAGTCCTGTAAGGTAAACAGG
ATAGGGCAGGGAAAGAAGCTGAGCCAAGATGTGGTTTGAGCTGAAGTCTA
GCTTCAGTCTGATCCAGTGGGATTTCTGGCCCATGACGATGCCAAGAGTT
TTCTCACCTTGAGGCA

An exemplary DNA sequence of human AmotL2 is provided below.

gi|224589815: c134097340-134071166 Homo sapiens
chromosome 3, GRCh37.p13 Primary Assembly

(SEQ ID NO: 305)

CTCAGGGGCCTCTTCACATTCCCTGCTGCCTCTTGCCAAGAGCAGAGGAT
CTTTCCAGGCAGCAATGGCACATCCACCAAATCAGTATTCACTCAGTATA
AGGTAGCTTTGCAGAAAAGGCAGGTGTCTGGGGTTACATTGATATTTCAA
AATGGTCTTCCTTTATTCACCGGCCAGAATGTGCCCATTCTTAGATCCCC
ACCCCATTCTTTCCCAGTCTGATGGCTACCCACACAAAAATATACTCAAG
TGTGCGCTCCTGCAGTAGTTCATCTATGTATTCACCAATATCCAGACACG
TCTTCCATGCAGGACTGGAAATTGTGAAGGCGCTGGGCAGTTACACACCC
TAGAAAAAGGGGAGGAAGTGGGAGCATTGTTCAGAGCTCCCTGCCCATTG
TGTAACCTCACCATTCACAGGAATCCTCTTAGCCCTGGGCTGGCAGGGGC
ACCAGAAAGAAGCAAAGGGCCACAGAGGAAGGCCAGGGGCTCAAGGAATG
TGTGCAGTTTGGAGAGGCAGGGCTTCTTCTTCTTCTTTTTTTTTTTTTAA
ACAGAGTCTCACCTGTCACCTAGGCTGGAGTGCAATGGAGCTATCTGAAG
TCACTGCAACCTCTGCCTTCTGGATTCAAGCTATTCTCCTGCCTCAGCTT
CCTGAGCTAGGATTGCAGGCGTCCACCACCATGCCTGGCTAATTTTTGTA
TTTTTAGTAGAAACAGGGTTTTGCCATGTCGGCCAGTCTGCTCTCGAACT
CCTGACTTCAGGTGAACCACCTGCCTTGGCCTCCCAAAGTGCTGGGATTA
CAGGCGTGAGCCACCGCACCTGGCTGCTTCTTCATTTTTTTAGGACAACA
TCCTCTTACCTAAACCACTGTGGGGCTAATTTCATAAATCTCCAACTCTG
GAGGATTTAACCCCCAAAGACACAGACCCCATGAGGCCCTTTGTGCATAA
CGGAGATTCCAGACCTGAAGCATGAGAGTGAAAGGGAGAGGGGGCAGCAG
TGGAAGGAGGAGACTTGGGGGAGAAAACCTAGAGGCAGTGATGAAGGCAG
GGGAGGAGAAAGGGGTGAAGGGGGGACAGGGGTTTGCAGAAGGACAAAGA
GAAGAAAGTCCCCCACTTGGGTCTTCTTCTGTTCTTGGAACTGGAGCACT
GGAACTTCCATCCTAATGTTCAGATTCTTCCTATGAATTTGCTTCATGCT
CCAGATTCCGGTAGGTTTCCTGTTTCCATAACCTGTCAGAGGGAGTGTTC
AGGGGAAGCTGGAAAAGTGAATTTCACAACTAACAGTCATCCTTTGATCT
TGCCACCTGCACCCTGGAGCTCTCCTCCACTAAGCAACCATTTGGGAAGT
AAGGGGAACATTTCCTTCCCGGCTCCATGGGAGGAGGCTGCTAGGGTCCC
ACCAAGAACTCACTTTCATGTTCTGGTTGGGTGCAGTGGGATGCAGCTGC
AGGGCCAGACTGAGGCCATAAAACAGGCACAGACCCAGACCGGGGCTAAG
GAGAGGCGATGGTGTGGGTGCCTGGATCCCTTGCTAAGCCCTGTACACAC
CTATGATGCAGCCACACCCCAAGGAGCTACACTTCCTAGAGCAAAGGCCT
CCAGCCTGGCTTACTCTGAGAATTAGGTCACCTCCGCATCTTTCCTGGAT
AGGATTCCTGACCCAGCCAGCCACATTGTCATCTGGGAAGAATCATAACC
TGTAGGAGGAAGAGAGGAGGTAAAGGAGAATGGGACAAAGTAGATTGAGA
CAAAGGTGGAAGAAGAAAGCATTGGCTCACTAAGAGAGCCTCTCTCCCCT
CTGCTGTAGGTGAAGATTCATATGCAAGCAGGCTGGCCGGCCCAGTGTCC
CCTGTGCACATCTGCCTCTGGCACATCTGAACAGTGGTCACCTTCTCCAC
CAACTATCCTGCAGAGCTGGGGTGGCCGCCAGCTTAGGTAGGCTCCACCG
CAGGCCATGCCTGACAGATCTTGCCTTCTGTTCTTTCTGCAGAGAGAAAG
GGAAAGGAAAAATAAAAGTACATGCACATTTCTCATTTAGGTTTTGGGAT
CCCTCTCCCGCTTCTGTCCCTTTGGGGACAGAAATTAGCAATCATGGGGG
CCTGTGTGGCTCTGCAGCAGGCTTGTCCACTCCAAGATGTACCTCCTTCA
CCCTAGCTGCCTTGAACTGACGCCTTTCTGCAACCATCTCAAGAATATTT
GTCAAACATTTTGTCAGGTATCCTCCCAAATGCCTGAAAGACAGGACTGT
ACCATGTGGTCTTTCCCCTGGTTTTACAACCCGAGTGCACATGTCCACAA
CCACTCCCCTGCCTCCACCAATTATGCTGTCCATAAGGTAAGGCCACTCA
GAGACCTCTTTCCTTTCACTTCTAAAATTCCTACCTACAGTGGTATACTG
ATCACAGCTTCTCTTCGTAAGGGGATCCTTGGAAAGACAAATATCCCGTA
GCTTGGCACAAATGATCCATTTGCTCAATCATCTCAGATCCAATCACTGT
TTGAAACACAGTATTTGACTTCCAAGTAAAACACACACGTGCGCACACAC
ACACACACTTTTTGTGAGTTTTATTAAAAATTAACAATGTCCCTGGCAAT
TAAGCTTGCAGAATTTCCTTGTCCTTGTTTCCCTCCAGTGAATTTGGGAC
TTGCAAACTCCATGCTGATGGGAGGACCCAGCCAGGGGTAGAAATCAGCC
CTGACTCTCTGGGTGGACAGGGGCCTGACTGCCCCCACCTCACCCCAACT
GACTTGTAGAGAGGGCTTGTGTGATGGGGAACGGGGACAGCATGTGTCAG
AGCGAGCTTTCTGAAGAAGGGGTTTCCATCTAGCAGTGGAAGTGTCCTGG
GGGCAGAGGAGAGGATTCCATGCAGGAGCCACAGAAGTGGGTACAGGGGA
AAGCCCCTGGCTGGCAGGGGTGTAGGGGGAGGTAAAGAAGAGACTAAAGC
AAGGACAGGGTAAGTGGAGAGAGGAGGGCTCCCAGTGTCTGCCCTGTCCA
TCTAGGGAGCCTGGATTCTGATAGCCAGGGAGGGGTTTTGAGAATGAGAA
ACCAAGCAGCCCAGTGGTTGATTACCTGGTTCTCACTGTGTGTGACTCTG
GGCACGCTGCTTAAGCTCTCTGAGCCCTGGTGTCCTCATTTACAACACAG
GGATGATAACAGAACTCCGTTGGAGGGTGCTTGGCAGGGCTGAATCGGAT
GACTGGGCGAAAAGCCAGCGGTGGCACACCTTGCACTCTGCGTAAAGGGG
CGCCTATTATCACCTTGGGGAAGAACTGGACAGAGCGCCTGGCGGCGGGT
GATTCAGTTGGGTAGGTGGGTCAAATTGACCGGATTTTCCAACCCAGATT
CACACTTCCTGTCGAGACGGTCGTAAAATAGCTGGCGAAGGTGTTTGTAA
ACAAAAGTGCATTCAAAGGTTGCCGCTGGGGGTGAAAACAGCGGGGGTTA
GGGGAAAGAGGCTCTGTGGCTGGAAGCATTCACTTAGCCCGGGGCACTGT
CCCAGCCCAGAGGATATGGATGTCTTTGCAGTCCCTCCTCCCTTTCCCAC
CTCCCCCGGTACACACACACGCACACACACACACACACACACACACAGCC
GGAGTCCCCTCCCGCAGCAACCCCAGGACACGCGTGGTACAGCCCTTGCT
GTCGTCCCGTCGATGGGTTTAGGGATGAAGGGTGAGAGCGGGTCGGCGAC
TGGGAGTGGAGGCGATAATGGGCAGAGTTGGATTAAATTGTCTCCAGCAG
CTCCGAAGGCAAAGCATGGGGCAGGGGCGGACACACCGCGGCCTGGAGGG
CGCTGGGCGCGGAGGAACGGAGCTCGGGGGCCGGGCCGGCGCTCGGCCAG
CAGCGCCGCCTCCCCGGAGGAAGGCGGGGGCCGGGAGGAGGGAGCGGGAG
AAGGCGGAGGGCGGAGGAGGGCGGTGCGCTGAGAGGTTATTTGTGGTTCG
CTTTGATCCCGAGGGGGAACCTGAAACTCTCACATTCCTGGCATAGCAGC
CGCCTCCGGCGCGGGCCGACCCTGGGGCTGCGCGCTGGGGCGCGAACAGC
CAGAGCGTCGGCGCCACGGCCGAGAACACATCTTCGCCGCCGAGCTGAGC
TGGGCCGAGCCGGAGGTTGTGGTGTCTGACTGCGCTGGGCACCCTCGGGC
CGCAGCGGTAAGGAAGGCCGCTCGGCGCTCGGGAGAGCGCGCGAGGCCAC
AGAAACGTGCCCCAGCCAAAGCTGGAGTCTCGGCGCAACCCGGCCTTTTC
CGGGAGTCGGAGCCTCGAGGATCGAGCGTCCTCGGGGCGCAGTGCGCCCT
TTCGAGGCAGCCGCAGTGCCTGGCAAGGGCCGCTCTGCCCAGGGAGCGCA
GCGCGCTGGGCTTTGCGGGGCTCTAGAAACGCGCGGGTCTCTTGGGGCTG
GGGGAGAGGGGAGAGGGCCGACTCCGGAGACAACAGCCACAGTGTTTCGG
GTTGCAGGTCGGCGGGGGTGGGGGTGGCGGGGGACAGGGATGGAGGCGCC
GCTTGGAGCTCAAGTTGCAGGGTCCTGCGGGCTGATTTGGTTAGGTGGGG
GTTACAGGTGCCTTTGCCCCCAACTAGTCCAAGAATTCCTCTTTGTCTTC
CTTGGAAAATCCTCTCTGGAGCCACGAATTGGCCCTCACTTAGAAAGAAT
GCAGCCTGCTGTCCAGCCCGGTGACGTCAGCGTTAGAGTATTTGGAAAAC
AAAGAGGGCTCGAGAGGGAGGGAGGCGGACCCGCGATGCTTAAAGACCTC
TCCGGGCCTCGGGCTACCCTCACCGGCTCGCCCCAGGCGTACCAGCGGCC
ACCGGTGCTCCAGGTCCGCCCGGGCTCGTGGTGGGGCGGGAGCCCTGGCT
GGGGTGCCAGAACCTCCTAGCTTGGAGACTGTCTTTGGTTCCTTAGGCGA
GCAGGAGCCCAGGCCAAGGCTGGAGACGCGCGCTGGCTTAGAAAGGCGGC
CTTCAGAGAGCGCAGCGGGCGAGTGCAGCAGAGGTTCGCGGCCGGGGGCG
CGGAAGCAGGTTTTTGTAAAGGCCCAGGCGTTTGATGTCTGAAAGGACGT
GAATCTGAGGGGCTTGGCCGGGACAAATTCGCGATGAGAGGCCTTGGAGA
TGTTCCTTGCTCCCAGTCCGGGCACACTATGGAGCGCCTGGAATCTGTCA
GGAAGGCTCCGCATTCCTTCCTGCCGCTCCCCTGCCTTCTCTGTGGGAGT
CGGCCCAGAGAGTGATTATCATAGGAAATGCCCGCAGATAAAACTCCAGG
GCGAACTGAATCCCATACGAATTGAATCCCATTTGCTTGCTTTGCCTCGG
GTAGCTCTGGAGTCTTGCACATCTACCCCTCGTTGTGTGTGGGGGGCGCA
TGACTGGGTGCCAGGAAGGATGGGTACAGAGGTGCCCTCCAGACTCATCC
CCCACTCCCCAGCTCTGGGCAGCTGGAGTACTGGGGTGGGGAGAGGGAAA
AGATCGGGAATGGCTGCTCTGCCAACATGCCATAGCCCAAACTGAAGCAG
GACACTAGGGCTAGTTTAGTGGCAGAGGAGGGGCCACATTCCTCCCACCC
ACCCCCCCAACCCCTGCCCATACACACAGGACCTGTGGCAGTGTGGATTC
TGTTTAGTTTTCCCTGGATCCCAGTTTGGAGATTTGTGTTGGAAGAGGCT
GTGGAAAGGAAGCTGGGTGAGAGAGGCAGGCAGATTTCCAGTCCATGAAC
CACTGGGAGCCCAGGCTTAGAAGGGAACTCCTTTCTCCGATATAATCTCC
CAGCACTTTAGGCTCACAGTCCCCTCTCCCCTGCCCCAGGTCTCTCAACC
AACAGCCTCCCCTTACTTCTCCAGTTGGCCCAAGTCAAGATGGGCTTCCT
TGACACTGATGTCAAGAAAGATGCTGAGTGCCCATCCCTTATGTGCCTAG
AGGACCAGTGGGGGCTTTTCCAAGTAGCGTGTGTGTGTGTTTGTGTGTGT
GTGTGTGTGTGTGTGTGTTGGGGAGGATAATCATAAAATCAAAGACATCA
TGCCATCCTTCCTTTTTCCTGGAGCTCTCTTGCAGTTACTGTCAGCACAA
AAGACTGACCCACTAACCTTAATGGGTGGCACCTTTTTGCCTCATCTTAC
AAAACCATTGTCCTCTTGAAGCATTTTTATTCATCCTTTATTCACCAATT
CAGGTGCAATTACTCCTTCTTCCTGTTCACCTTGCAGAATGACTGCCAAG
GTCATCCCATGACAGGGAGCCCCAGATTCATTCTGTCAGCAGCGCTTGAT
GAAGCAGAGCCCAAGCCCATCCTCCAGGAGTTCTCTGGTTCAGGGAGAGT
GGGAAAAGTGGCCAGGGGTTCAGCGGGCTAGAGGGTGGTAGGTTGACTGT
GCCAAGGCTCTGGGATGCCTCAGTCTAGAGGGTGGTAGGTTGACTGTACC
AAGGCTGGACTCTGGCATGCCTCAGTCTAGAGGGTCCTAGAGGCCTTTCG
CCTAGGGCTTCTACTGGACTGCATGCATTCTTTATCACCTGCCAATAAAT
TGGGGCCCCATCCACCCCTAGACTGAATAGCTAAGTGGTTAGATCTGAAC
TAGGCCTGCCTTAGAATTGCCCATCAGGCTGTTTGATGTGTAGCTGGCTG
TACGGGTGGTGGTTCCTGGTGGGTTATCTTCTGGTTTCCTGTTGTCTCTC
TCCAGGAATAGTAAGTCTCCATGAATGTTATCATTAGTAATCGTTTACCA
GGATTTTCACTGTGCCAGGAGCTCGCAGGCCACTGCCTCTTTTTTTTTCC
TCATCTCCATGAGTCAAGCAGAATTCTTTGCATTTTTCTGGTGGAGAAAC
TGAGGCTCAGAGATTAGGTAACTTGTCTTAATCAGTAGATGGCAGCACTG
GTACTTGAACCCAGGCTTGTGTGAACCGCCCCACCCCTGCTCTTCACTTT
TATGCTTTCCACCAGAGTAATAATGGAAATCCTGGAAAGCCTTCTCCTTT
CCCATGGGTTCCCACTGATTGCTTTTCTCTCTCTCTCTCCCCACCCCACT
CCAGGTGCTCTGGGGCCAGGTGCCACCGGCCATTGTCCAGGCAGCTGTGT
GCAAGCCAAAGAAGCATGAGGACACTGGAAGACTCCTCGGGGACAGTCCT
GCACCGCCTCATCCAGGAGCAGCTGCGCTACGGCAACCTGACTGAGACGC
GCACGCTGCTAGCCATCCAGCAGCAGGCCCTGAGGGGTGGGGCTGGAACT
GGGGGTACAGGGAGCCCCCAGGCCTCCCTGGAGATCCTGGCCCCAGAGGA
CAGTCAGGTGCTGCAGCAGGCCACCAGGCAGGAGCCCCAGGGCCAGGAGC
ACCAGGGCGGTGAGAACCACCTGGCAGAGAACACCCTCTACCGGCTATGC
CCACAGCCCAGCAAGGGAGAGGAGCTGCCCACCTATGAGGAGGCCAAAGC
CCACTCGCAGTACTATGCGGCCCAGCAGGCAGGGACCCGGCCACATGCGG
GGGACCGAGATCCCCGTGGGGCCCCGGGAGGCAGTCGGAGGCAGGACGAG
GCCCTGCGGGAGCTGAGGCATGGGCACGTGCGCTCGTTGAGTGAACGGCT
CCTTCAGTTGTCCCTGGAGAGGAACGGCGCCCGGGCCCCCAGCCACATGA
GCTCCTCCCACAGCTTCCCACAGCTGGCCCGCAACCAGCAGGGCCCCCCA
CTGAGGGGCCCCCCTGCTGAGGGCCCAGAGTCCCGAGGACCCCCACCTCA
GTACCCTCATGTTGTACTAGCTCATGAGACCACCACTGCTGTCACTGACC
CACGGTACCGTGCCCGCGGCAGCCCGCACTTCCAGCATGCTGAAGTCAGG
TAACTCTCCCCACCTTGGGCTTTCCAACTCCTGGCTTTCTTCCAGCACAG
GTCTCTCAGGGAGGAGAGCCTCAAGATCACAGCTTGTGTCAGAGCTAGCA
AGATCCACCCCACCTCCAACCTGAGAAGGGAGAGCTAATGCCTGGCAGAG
AAGAGGGACTTAGCTGAGGTCCCCTAAGAAGTTAGTGGTGTCCCTTGCCT
TAATTCAGAGATAGGCACCATTGTCTGCTTAGCTGTAGCATTCAAGTAAC
TAGAGTCCCCAGAGTACACAAATTGCAGCAGGGGGTTAAAGGTTAGATTT
CAGAAAGAACTTCCTAAAGGCAAGGTTGGGAATGATTGCCAAACTGTTTC
CAAGTTAAGCAGGAAGAAGGTGGGACTTCCTTCTCTGGAGATTTTTTAAG
AAAATATGAGCAGCTTGCTTTCTGTGATTATTGGGAGGCATTCCTAACTA
GAGGCTGGAAAGGCCCAGTGGTCTGATTAGAGACCTCTCAGCATCTTACA
CCAGGGCAGGAGCTTACTTGTCTCTCCTCTTCATTTCCTGCCATATGCTC
CCTGCGTCTGCAGCCTGCAGTGGGAAAGGATTCTGGGTCCAAGTGGGCCC
TTTTCTGGTGAGGCTCAGGAGAGGCTGCTGGGCCATGGTAGGGACTTAGA
GTTTGATTCTGAGTGTGCTGGGAGGCCCTCAGGGGATGTGATTTGGTTTA
CCTTTTCTAAATGCTCTCTCTGGCTACATGCAGAGCATGGTTTGTAAGGG
GCCAGAATGGAAGCAGGAAGACCCGGGGAGGCAAGAAGTCTAGAGAGTTT
GGAGAAATAAGTGTTCCTAGAAACAAGGCCCTGCCTCACGCTTCTCATCT
TGCCCAGCATCTTTTCCAGAGAAGCAGACACAGCCATCACTGCTGCATCT
CCCCCTATGGGAGTCCAAGAAACCCCACCAAGGGACCCCAAGGATTTCAG
ACTCTTCCTCTGGTGGCCCAAGGTCAAAGTCTGGTTCTCTTCATGCCTGC
TGTGTTCCTTTAGTAAACACCCACTTTCCCACCGTCCGGCTTTCCTTGCT
CTCGAGAAATGAGTGACATGATGGGGTTGGTATGGCGGAACCAGAGGTTG
GAGCCTCATAGTTTTTCTCCAGCCTCCACCCCTCCAGACTCGCACTTTAT
CGCCCCTCTCAGGGCTCGGTCAGGAAGAGTTTCGGAAGCTGAGTGGTGGT
TTCTTCTTTTGGACCAGCCTTATTGAACCCTGTCAGGAAAAGATAGTAAT
CACGGCAAGGAAGTAGACTGTTTGCAGGAGAACAAAAAGCAGGGGAGGGA
GAGGACGAGGAGAAAGGAAAGCAAGTGTGAAGGGAAAGGAGTTGCTATTC
TTTCTCAGAGCTAAGTCATCCCCAAAGAATGCCAGTTGTGTCTGCCTGGC
CCAGTGCAGGGGGGAGCAAGTTCTTGAAGCAACCTTGAGAATGGCCATTT
TAGCAGTGGAATGGAGAGTTTGAGCACTCCAGTGGGGACCCTCTTCCCAG
CTCTTACTGGAAGCCCCACCTTAGGTAGTAGGAGAAATACATCTGCCCAA
TATCTCCTTTCAGATGATCCAGTGAAGATTATTTGTTAGGAAACGGGCTA
GACTCCTAGCCTAGGGTCCTGAGAAGAGCAGGGGGGCCATAGGCTGCATG
GCAGCAAGAGTCAGGGGCAGGCTGGGTGTGCATTAGTGCTGAGATGATGC
ACCCAGAAGGGGAATTCGGTTTTTGCGGGGCTTCTGGGACTTGGAAGATT
CTTTGTGAAACTGAGGCAGTTGTGACACTGGGAATCACAAGGTCCCAGGA
AGGCCCTTGGACTCCCCGTGCCTCTTGCCCTTTGCTCCTTCCCTCAACAT
GGAGCCCTGGGGGTGATGTTACACCAGTGCCAGAGGCCAATAGAATATTG
TCCCCTGAACTCAGTGACCACAGTAGTCCAGAGGCAGCCCTTACTCCCAA
GGAAACCAAGAGTTTGGGCCACATTTGTTCTCTAAACTGAGGGTAAACTG
AGGCTCCTACTAGAAGAGATGGAGGATGGGCCACCATTATAGGAGCACCT
GCCACCCGCCCAGCACTGGCCCAGCACTCTGCCAGGCCTTACATAGCGTC
ATCCTCAACAAATCCCAGCGAGGCTGGAGACCCTCATGATGCAACAGAGC
AATGCCCAGTCTGTGGGGCTGAGGCACTCGCCCAAGGTTAAACAGCTGGA
GCAGGGATTTGAACCCCTATTTGTCAGACTCTAGAGCTCAGACTCTTACC
CTGACATTTGTCTCATTGCTCTAACCTTAACTGGAGTCTTCTAAAAATGA
CTCTGGGTGAGTGGGGGGTGGGGGGTGGCAGGACTTGAAATAGGAAGGAG
AAAATGAGTGCTGCTTTGGCAGTCTTGAAACTGGCGACTCGTGTAATAGA
CACAAGGTTGCTGCTGGGGCCCAGCCACCAAAGTTGTCCTGGGGCCTGGA
AGGTTATATGCTCAGTCATGATTGACATGGCCTCCAGCCCCAAAAATAAC
CTTCCATTGATTAGAAGTGTCAGGGTGCATTTTTTAAGATGATGTATTTA
AGCATTAGTTGCTTAGGAGGCCCAGGATTGCTCAGGCTGATCCCCAAGGC
CCCAGCCACTGTGAGGCGGGTATGAAACAAGCATCCTGACATTAATCCCC
TCTAGCCCTGCTGTCTGACTCTTGAGAGCCTCAGGCAGAAACATTTGGAA
AAGGCCTTCCCTACTGGTCCCTGCCTAACACTAGAGGGTTGAATAATTAT
CTGCAGTAATTTTAATAATATATTAATTATTGATAATAATTAACCATCTT
CCCAGTATGCCTAGGAGTGGGGTGACCCCGGGCTGCTCTGGTGGGAGCCA
TGAGGGTCTGTGCTGTCTCTGAGCACCGTCTCTTCTGTTCCCCAGGATCC
TGCAGGCCCAGGTGCCTCCTGTGTTCCTCCAACAGCAGCAGCAGTACCAG
TACCTGCAGCAATCTCAGGAGCACCCCCCTCCCCCACATCCAGCTGCTCT
CGGCCATGGCCCCCTGAGCTCCCTCAGTCCACCTGCTGTGGAGGGGCCAG
TGAGTGCCCAGGCCTCCTCAGCCACCTCGGGCAGTGCCCACCTGGCCCAG
ATGGAGGCCGTGCTGAGGGAGAATGCCAGGCTGCAGAGAGACAATGAGCG
GCTGCAGAGGGAGCTGGAGAGCTCTGCGGAGAAGGCTGGCCGCATTGAGA
AGGTAGGCCCTGCTGGGGCTTCAGAAGGGCAGACCAGAAAGAGACCCCGA
GGGGCTACCTGGGGAGTGGGTAGTCCAAGGAGAGGCAGAGGAGCCTGAAA
ACTGTTTGGCTGGATTTCTCCCTATCCCTCTGTCTTCCACCTCCCTCACT
CCTCATGCACCCTCCTGAGCTGACTCCAGACAGCTGGGGAACAGCCGAGA
GGCCGGCAGAGCAGGCCTGGAGCATGAACAGTTGCTGGCCAGTGGTGATT
AGAAAGAGCCCATTCACCAGCTCCTTGCACACTCGCACCCTTGCCCTGCA
CACACCCCCTCCCATGGACACCTCCCACAGAGAGCCCTTTGTGTCCCCCA
CACAACAGCAAGTCTGTTCCAGGCCAGCCCTGGCCAAAAAGAAAACTTCT
CCTCTTGGAAAAAAAATGGGCGACTTGTTGGAATCACAGGCCAGCTAAGA
GCACGTGCGGAACCTGCCAGCAGCCAAGTGGGAGGAGCAGCCGGGGCCCA
GCTGGCCCTCTGGGTGGATGGTGATGCCTGGGACACCAGGCAGAGGCAGC
TCTGCCTGGTGCCCGCTTCTTTAGTGAAGGGTTACCAGTTTTCCGCTGAG
GCTCAGTTTTAGCAGATGGCCTCAGGCCTCCCTTGACTGTCCTGTGGTGT
CCAAGTTAGGTCAAACCACCCCATCATGGCTGGAGTCTTTCGGTGCCCAC
TTCTGGCCAACTGTACTCAGTTTCTTGGTGGAAGCCAGCCAAGAGTAGAA
TGTTTCCTTCTGGACACTGGGCACAGGGAAGCTGTGTTTAGTCACCCCGC
AGGCAGCTACTTCCAAAGTACCTGGCCCTGGGTTCACTCAGCATCTGGTC
CTTGGGGCAGCCGATGGGACCGTTGCTTGCTGGTCCCAGCAGTCTATTCT
ACTTCCGGAGGCTACAAGCTTCTGACCATCCCACCATAGTCACCTGGAGG
TTTTGCTCCTGTTTGGTGGCCTCTCATCTGTCACCTTTTCTGATGGAGAG
TAGTGGGTCAGCCTCGAATCACTCCCTGGGAGCTCTCGCCTTCATTCAGC
TCTGCTGTCTGACTCTTGCAGCTGGAAAGCGAAATCCAGCGGCTCTCTGA
GGCCCATGAGAGCCTGACCAGAGCCTCCTCCAAGCGTGAGGCCCTGGAGA
AGACCATGCGGAACAAGATGGACAGTGAAATGAGGAGGCTGCAAGACTTC
AACCGGGATCTTAGAGGTGAGAGCCAGTCACACAGGTGGAGGTTGGAGAA
CCTCTGCAGAGGACTTACTTCTTTTGTTCTAGAGAAAAATTGGGAATCTC
TATTCTCTGTCAAAAGTGCAGATGAACTCAGAGAGCCAGGCTGAGACCCC
TTTGGAGCTGCCCTGATAGAGAGATGCTTGTCTCTATGTACTGGGCCTAG
GATCCCCTCAGGGATGATGGCTCTTGACAGAGGAGGCTCCCACCCATGGG
GCCCTGAGCTGAGCTGGGGTTTCCTTATGAACCCATCCCCAATATGGAGG
ATACAGCCCCATCCTGATAGGACGTCAGGAAAAATCCCAAGCTGGCAGCT
TCACCTGACACTAAAACATCTTTCCTTCCCTTGAAACAGAGAGATTGGAA
TCTGCAAATCGCCGCCTGGCAAGCAAGACACAGGAGGCCCAGGCCGGCAG
TCAGGACATGGTGGCCAAGCTGCTTGCTCAGAGTGAGTAGGGGCCCCACT
AAGAAGCCTGGCCTGTGTGCAGGCAATGGGACTGCTGCACACCCTCTGGA
CTAGGAAGATTGGCCTTCCCCCTTGCAAAGTAGTACTTTGGGAGCAGGTT
TACTGCCCACTGTATGCCCAACACTAGACTGGATCTGGGGATAGTGGGGA
TAAGTGGCTTCCAAAGAAGAATAGGATCTAGTTCTGTATCTTTTAAGAAG
ATCAGCTGACACAAGAGGGAAAGCCAGGGAGCAGTGATTTGCTGAGCTTC
GGTGCTGACCACCAGTGCAGTGCATTGGGCAGTGCGGGAGGCAAGGTAGC
CCTGAAGACAGATGAGGTGAAGGAGAAGGACAGGTCTGAGAGCTTCCCAG
GGAGTTCCTGGCAACAGAAAGGTCGGGAGTGTGCAATGGGAATGTGAGAG
GTAGGGGGGCATGGTGGAGCTGAGCCATAGCAAGCCAAGTGGAGGCATCT
GAGGCAGCCAAAAATGGGATCCTAGACCTTAACCAACAGATGCATTCTAA
ACGGGGCGGTCTAAGCATCCTTGAAGAATGGGCAAGTTCATTGACTAGTC
CATTTATTCATTCATTAGCAGATGTGTTTTAAGCAACTGGCAGATGTTCA
AGAGAGAATGGGAGCAGGCCCAGGAAGGTACCATGGCAGGGAGGTCAGGA
AGGAAGTGGTGAGCTTAAGAAAGGAGCAGGTTCCCACAGATGCAAAAGAA
CAACGGGGGTGGCCCTAGAAACCGGTGATTAGGAGCCCCGGTGGCCTGAG
AGAGGGGCTCTGTGGGGATGGCAGGGGTGGAGAGCCAGCCAGAGGTGAAG
GAGCTATTGGGCAGTTCAAGAGGTACATGGGGTGGGTGGCAAAGGGAGCC
CTGAGCTCAGCAGCAGGAGGCTCTAGCAATCAGAAGAGACTAAGAACAGT
TATTTATTGAGCTCCTACTAGGAGCTTTAGTGCTGTTCAAGGTGTTTGCA
GAGAAGTGGAATGGGAGTGGTTGGTAGCTGGGTAAACATGGCTTTGAGTC
TTCACAGGAACCCTACTGTGAATGGCACAAAGGGTAGTGTGGGGTGCAGA
AATGGAGGAGCAGCTTGAGGACTTAGCTCTGGCTGGCAGCACCCCTATCT
TCCTTCAAAGAGGGTTCAAAGACTAGGGCAGGGAATGGGGGAACACAGCA
CTGAAGGAATTGACAGGGTGGGGCAGGAGGTAGGCTGGTCTCTTCTGGCT
GGGTGGGGTGAGATGGCAGCGGCTTAAGGTCTCTGGGAAGGAGTCAAAGG
GAAGGAGGGAGAAAGGGGAGGGGGCAGCCCTGGGGTTTCCCTGAGGAGTC
TGGGGAGGAGAAGCTGCAAGGGCACGGCAGATTAGGGTGCCTGTGGAGGA
AGGCGGACTTCTGGAGCCTGACTCTGCCAAAGAAAGGAAGCCATGGGGCG
GGTTTCTGGGAGCTTGCTAGTGGGTGAGAAGAGGGGGGCCGGCCGGGAGG
CGTTTCCGCTCCCCGGGGAGCGAGTGAGCGCCGAGCTGTTCTTGGCTCCT
CGCTGGAGCCATTAATCTTGCAGCCGCGGGTGACCTGGATGCCTGGCATT
CCAGAGCCGCGAGCTGTTCCGCTCCCGGCCCGCCCCCCGCCGCTCCCTCC
CGTGCGCGCTGCCTGGCGCCCTTCGCCCGGCTTTGGGAGGAGGGAGGGTG
AGCACAAAGGGAGCCTGGAACGTGGAGGTGGGGGCTCGCTCGCTGCCCAA
GGCGCATCCCGGGTCCCGTCGGCGCCTCGCCTGCCTTCCTGCCCTCCTCC
GCTGGCCTTCGGGCGTCCTCTTCCCACCCGCACCCAGACCACTTATTTCT
TACTAAGGAGCTCTCCTTCCCGAGATCCCGCAGGCCCAAGGTTTTATCAG
GGTCCTGTTGGTAGGGGATGGCCAGAGCTCAAAGCCTCCCAGCCCCCACC
AGGGCCTTAGAGGTTGGACAAGGCTGCAGGAAGCTTCTGGAAGCAGAGGG
CTGGGTCCTCCGGCCCCTTCCTCCCCCAGGGCTGAGGAAACCCAAGCAGC
TCTTCCTTGCTCCCTAGTAGCTATTGCAAAGGGAGGTGTCTGATTGACCT
CCCTTTTATCTGGGGTCAGTCAACACTAGGAAAGTGTGGCCATTATCACC
AATGCCATCTGCTTTTCCTCCTAGGCAAGCCACGCAGCCCCCCAGCACTG
CCCTTCTGGCCCTTCTAGGCCAGCAGTGCCCCCTGGGGATCTGGCCTTGA
GGCCTGAGGCTGGCCTGTTAGCTCCACCCCCTTGGGAATATTTCTTCTCT
CCCTGCGCCCCAGATTCTCCAGCTATAAGATGAGACAGAGTCCTTCCCCA
CAGAGTCATGCTAAGGCTGAAATGAGATGCAGGCCCTGGGCAAACTGAAC
GCACAGGTGGACTAGGGGAGGGTCCTCCCTGCCTGAGTCCCAACCTGGCT
GCCCCTCCTCAATGGATGTTAACACCCCACTCAATGGATGTTAAGCCAGC
GAAGTATGAACACATCCGTTTCAATATTATTGGACTTCCTCGTATTTTCC
TCCATGCCACAGAATGATACCATCCTGGGCTCAGCCTTCAAACATCCAAA
ATGAATCAAGAGCTAACCTGAGTTGCCTCTGTGTCCTCCCTGCCCTGCCC
CAGGGACCTGAGTAAATGAATCCTCCCTTCCACATGCCAAGGTCTCAAGC
CTTCACCCTTCTGTTCCAGAACTTGCACTGTCCTATATGATAGGTACTTA
GCTGCATGTGGCCCAAAGATTGAAATTAATGAAAATAAATTAAATTTAAA
ATTCAGTTCTCCCATCTTACTAGCCACATTTCCAGTCCTCAGTGGCACGT
GTGTCTAGTAGCCACTGCATTGGAGGGTGCCGTATAGAACAATTCCACTC
TGGCAGAAAATTCCACTGAATCAGAATGCTGTTCTAGAGACAGTGCCTTT
AGGTTCATTAGCGGGAGCCCATAGCCCTGCCCCCCACCTGCTCTCCAGCA
AGGTAGGCAGCTGACCTGCTCCTCAGGGGGCAGCCTGGGACTCTCAAGGG
GCAGGTGCCTGGGAGGTGGATTTAAGGCTAGAACATCAGATGGAGGGCTC
TGTGGTCTGGCTTGGGCTTCTCCTGGCCCTATGGAGAGGGCCTCTGAGCA
GTTTCTTTGGGCAATTAACAAAATGATTTAAAAGTCAGGATAGCTTGACC
CTTACACACTAATCTTTGGGCCATAGTCATTTTCACTGTACTGCAGACTT
TGCATTTCCTTCTGAGCCTGGTTGCTTACATCTCAGTGTTCTTTTCAGAC
CATGGAAGGAAATGTAGCCCTCAACGGACCTCTCGGCCTGGGTCTGCAGA
AGGAGCCCCAGAGCTCTGGGTGGCCTATAAGTACAAACGCCTCACCATGT
AGCCAGAGGCAGCCTGGGCCTTTGTAGTGTGGCCAACTGGGGTTTCCATT
TGGCAGCACCATTCACCTCCTATGTTTCTCCCCTATAAGTTGGGGATCCC
AAATCCTCCCTGGAGGGGCTGGAGGATTCGTGGAATAAGATGTGTAAGGG
CCTGGTGTGCCGACAGCCGATGGTGGTTCTGTTCCCCCTTCTTTCATTCT
CCCACTGGAAGGAGGACCATGTCACAGCCTACCTGAGTCTCTATTTTCTC
ATCTGTACATGGGCATCACAGTTCCTACCTCAACAGTCTCGAGCCGTCAC
AAGGTTCAAGGGATATTGTGGATGTGTTTTAGACATGGTGCCCCTGGCAC
AAGTGGATGGTGTTTGGGGCTGTCCCATGGCAGTATGTCCCAGTAGAAAG
GCAGGGTAGCCCAGTTGTTACTGCAAGGGTCCTGGAATTGGGCTCTGTCA
TTTACTCCTGACTTAACATGAGCCTTGGTTTCTTCTTCCATGAAACAGGG
TTAATAGTGGTACCTTATTCCTGTTATTTTTGAGAGAATGAGAAGCAACT
GTGCTCATCCAGTGGTCAGCACTGAGCCCAGGAGGCAATGGGGCCAGCCA
ACGTGGTCACTGTCACTGATCAAGCCTCTTTCTCCCTACAGGCTACGAAC
AGCAGCAGGAGCAAGAGAAGCTGGAGCGAGAGATGGCACTGCTGCGCGGC
GCCATCGAGGACCAGCGGCGGCGTGCCGAGCTGCTGGAGCAGGCTCTGGG
CAATGCGCAGGGCCGGGCAGCTCGAGCCGAAGAGGAGCTGCGCAAGAAGC
AGGCCTATGTGGAGAAAGTGGAGCGGCTGCAGCAGGCGCTCGGGCAGCTG
CAGGCAGCCTGTGAGAAGCGGGAGCAGCTGGAGCTGCGTCTGCGGACTCG
CCTGGAGCAGGAACTCAAGGCCCTGCGTGCACAGCAGGTGAGCTGAGAAA
GTCTCCAGGCTAGGGCAGCATCTGAGGATGTTGGCTTCCCTTCCTCCTCG
GGGACTGTAGGTTTGGAGGCCAGGGCTGAGGCCCCGGGAGCCCCTGCTTA
GATCTAGGCTGCAGGCCCGTTGGAAGGCGGCGAGGATGATTGCTCTCTGC
TCCACCTTTGCTGCGTGCTCCCTTCCACTCGTTGTCTTAGTAGAGGCCCT
GCCACCTGGCCCATGTCCAGCCTCGGGGGACTCCTGTCCTCGGCAGCAGC
AGGGCTAGTTGTCCTTGACCTCTAGAGAAAGTAAGTCAGGAACCTGGATA
TAGAAGGCCTGCGAGCTTTTTTTTTTTTGAGATGGAATCTCATTCTGTCG
CCCAGGCTGGAGTGCAGTGGCGCGAACGCTCACTGCAACTTCCACCTCCC
AGGTTCAAGCCATTCTCTTGCCTCAGCCTCCCGAGTAGCTGAGACTACAG
GCGTGCGCCACCACGCCCAGCTAATTTTTGTATTTTTAGTAGAGATGAGG
TTTCACCATATTGGCCAGGCTGGTCTTGAACCCCTGACCTCACGTGGTCC
GCCGACCTTGGCCTCCCAAAGTGCTGGGATTACAGGCATGAGCCACTGCG
TCTGGCCACCAGCCAGCTTTTATATGCCTCCCCAAGATTGGGAGGAAAGA
TGGTTGTCCAGATCCACTGTGCTTAGCCTCACTTCGGACTTCTGTGTAAA
CAACTCTTGGTGTTTACAAACCAAATAGAGTTATTTCTCCACTCCCAAAC
CTACCCTTGCCACAGAGGTAGTGTGACTGAACCCCCTGGTTTGCCCAGTT
CTGTTCTAATGTACACCAGTTTTCCCAGTATAATTATTGATAGCACCCCC
TTTGCCAGTTTGGAGGATTATCTGTCTGGTCTCTATGCCCTTGCCCTAGG
ATTCAGGGCAGGGGTATGGGGACCTGTGCTGTATCCCCATTTCGGTAGAG
GAAGGATGGGGAAATGTATGCTGTGTATGGCTGAACACGTACATGTACAA
GTGACAGTCGTGTGGAGGCGAAGACCAGTGGCTGCCACACCCCAACCTGC
ACCCTTGACCACGGTCTCTACTCCTTGCTCTGCAGAGACAGGCAGGTGCC
CCAGGTGGTAGCAGTGGCAGTGGTGGGTCTCCAGAGCTCAGCGCCCTGCG
ACTGTCAGAACAACTGCGAGAGAAGGAGGAGCAGATCCTGGCGCTGGAGG
CCGACATGACCAAGTGGGAGCAGAAGTATTTGGAGGAACGTGCCATGAGG
CAGTTTGCCATGGATGCGGCTGCCACGGCTGCTGCTCAGCGTGACACCAC
TCTCATCCGACATTCCCCCCAGCCCTCACCCAGCAGCAGCTTCAATGAGG
GTCTGCTCACTGGTGGCCACAGGCATCAGGAGATGGAAAGCAGGTTCGGC
ATTGCAGGCCAGGCACCTGAGGTTGGGGGGGTGGGTGGCAATGTCCAGTC
ACTGCCCTGCCCTTGATCCCTGATAAACAGGCCCCTTTCCCCATAGAGCT
CAGCAGTCCCTCCTGCTTCTGAGCTGAACCTGCATGATTGGCACACCAGG
CACTGTAAACATGCATTTGCCCAGAAATACCGAGTCCTTGGTCTGCCCAG
GAGACTCTTACTGTGGAGGTGTTGGGTGCCATCCAGTGCTTGGGCCCTGC
CTCCCCTCTCCGGAGTGCCGCCCTCCAGGATGAGGGCTGTGCCGCCACTA
TCATCTGATGAATCTGGAACCCATCCTTAATGGCCTGCCTTTGCTGGTGT
GAGAAGGGCCCAGGGCCTTATGCACTGGTGGTTTTCAGAGCCTGGGCTCT
AAGAGCATCAAATTCCAGAGCTGTGGTGCCATTTCTAAGCCCTCTCTGGG
TCCCTGCTACAAGCACAGAAGGATCCAGCCTGGCAGGGTTGGGGGAGTTG
GGGGGGGCTTTTCCTCTAACTCCTATGAGGCAGGAAGGGTGGAGGCTGTT
GGATCTGTGGGTCTGACAATGTGGGCATGGCCTCTCACCCCTCAGGTTAA
AGGTGCTCCATGCCCAGATCCTGGAGAAGGATGCAGTGATCAAGGTCCTT
CAGCAGCGCTCCAGGAGAGACCCTGGCAAGGCCATCCAGGGCTCCCTGCG
GCCTGCCAAGTCGGTGCCATCTGTTTTCGCGGCTGCGGCAGCAGGAACCC
AGGGCTGGCAAGGGCTCTCTTCTAGTGAGCGACAAACAGCAGACGCCCCT
GCTCGGCTGACTACAGGTAAGAGGAGGCTACTGCTGGGATAGATTGAGAG
GTAATTTCTCCTGGGCCTGAACACAGATTGCCAGACCTTTCCTCCCAGAA
CTGAGGCTATAGGAGCCAGGGCTTCCTGACCCTCTTCCAGGGGAGCTGGC
TTATTCTCTGCCTCTAGTGACTCTACAGCGCAAGGCTCTCCTGCCAGTGT
GAAGAGGGGTACTTAGTCCCCATCACCATAAATTCTATCATGCTTCAGCC
CCAGCCACTGAACTAAGAAAAATAGAGCTTCCTGTGCAGATACACCTTGT
ATGCAAGCTCCCTCCACTCTTCCTTCTAAGCAGATGCCACAGAAACTCTA
GAAGATTTAATCTATAAGGAAGGGTCCCACCACCCAAAACATTTGGAAGC
CAATCCAATCTGGTAGTTATTCCCATTTGAAAATAGGAGCATTTTTAGTC
TTAAAGTTCTTGCAAATCAAGAGCAGCCCACTCCACCTTGAATCCCCAGC
AGGGACAGTTAACTTAGAGTGTCCTTCACCAGGGCCACATCTTACAGGCA
TGGCAATAACCATCCACCTACCTTTCCAGCAGACAGAGCACCCACAGAGG
AGCCAGTGGTCACAGCTCCCCCTGCTGCCCATGCCAAACACGGGAGCAGA
GATGGGAGCACCCAGACTGAGGGCCCCCCAGACAGCACCTCCACCTGCCT
GCCACCGGAGCCTGACAGCCTTCTGGGGTGCAGCAGTAGCCAGAGAGCAG
CCTCTCTGGGTAAGTCCTCCTGACCCCACTTCTGTGCTAGGTAGGGCCGA
GGGGGCACTGGGGTGTGAACTGGAACACAAGGCCTTTTCTTCCCAGACCC
CCTCCGGGTCATTGCCCGCACTCCATGGGGAGCCGTGCATAGCCAGAGGT
GAGCAGGGTGCCTGGCCCACCTCAATGTGAACATGAGGGATGGCTGGTTG
CATGAATAGGGAAATTGGAAACCCTGATTAGAGGCAGATCCAGAATTCAG
AAAGAAAGCAACCAGATGGGAGGCACTTTAGCTCCTCTTTCATGGTCTTC
CCTACATAGTTAATTTCCAATTTGATTATCCAAGAGAATGGAGCTCGCAA
GTCATGCCGATGGCACTCTGGAAGCCTGTTTAAAGAGTCCGGGAAGGAGA
AATCTGAGAAGATGCCATTTCTTTTTCCACTCCCCTTTCACATTGGCCTT
TGCATATTTGCCTGTGCCTCATGATCTCAGAAAACCACAGGCATGAAAAG
AATGCTGCAGCCTGCCTCCCTCAGGGCTCTCCATTGCTCTCTCTAAGCCC
TCCAACCAGTTCAAAGTTAGAAACAAATGCATCCTACACATCGGAGGTTG
GTGGGCTGGGCTGAGCACTGAAAGGGGGCAGAGCATTGTGCTTGGTTGGC
CGAGTTCTGGGAACCTGGGCAGCAGAGGTGGGAAAAGTTGGTGAAGAACG
ATCTTTGTCTTTCAAATGTGCCAGGATTTTAAAAGCAATCTAGTAACACT
CATTGGCATGTGCCTGTGTTCTTGTCTGTCTCCAGACTCTGTAGCTACAT
CCAGAGTCCAGGACTTGTCAGACATGGTGGAGATACTGATCTGAAGGAGG
TGGTGCTTCAGGACTCTGAGCCATTCTCTCCCCTCCTCTGCCCTGTGCCA
CTCTCAGCCATTTCAGCAGCCCCGTCAACCGCTGCTCCGTCCCTTTCCCC
AGCCAGACACTCATTCCCATTGACCATCTGGTCCCAGGAGCTCAGGAGGA
GGACCCCAGGGGAGAGGAGAGCTGTGAGAGCACCGGCACCCCCAGAAGAC
TCTGCTTCTTAGCCCACATTCCTCCGGGCCTTATGGAGAATGAGGATTCA
GCCTTGACTTCTTGCCCAAGGCCTGCTACTGGGGTAGCAACTGACAGCTC
AGAAAGGAGCTGAGCTCCCTCTGCCCTGCCAGTTGTCAGTCAGGCAGGGA
GGGAGTGGCTGTGTTGGTTTGGGGAACTAATTTCCACGGACGGCTGCCCG
TGGACACCAGGTGGACTGGTTCACTAATCAAGTCAGCCATATTGTTCTCT
GGCTAAGTTTGGTTCCAGCCAACGTCATCTGCTCTTCAGTTCCTCACTGC
CTTCTTGGGATACTAAGACTTGAATTTTTTGGGGACTATTAAGGGTGTTA
GTCTTGGAGAAGACACAGCCTCACCTTCTCACTTGCTGTGGGTGAGGGGC
CATTTAAGTGGACTGGGAGACAGTGCGCAGTTTGTATATAATTCCCTTTC
TTGTGGAACAGAAGACTGAGGCCTGCAGGTTCCGATGTGTCTCCATGGGC
TGTGCTCCCCTCTTCCTACTGTCAGTTTCTGAAACTTCTGACTGGCCTCC
CAGTTATGCCTCCTCCTCAAGTTCCTGGCCCGTGGATGTTAAAGCTGCTC
GATTCCCAGGATCTCGGCTGCCTTTTCCTCTATCTTGAGCCCTATAAATG
CCCACGGGACCCCCACCACCAGCCTCTTGAAGTGGCTCCACAGCTCCTGT
CCCTGGAACATCCTGTCAGTTTGGTCATAAACCCTGAGCCAGATGAAATG
AGCCACCGTGAACAGACATCTGCCATGCCCCCAGGTGGGCTTCGGTGGCC
CTACCCGGTACCAGTTCTTTCTGAGAAACTGGAGATGTCTTGTTAGCATA
AGTGTCTTCATTCCCACCTGGAGGGTTTGGGAGAGGAGCAAAGCAGTTGA
AAACTAGTTAATGAGCTACAAGAGTCAAATAGTCCTCTGAATGGAGCCCC
CATCACAAAACAGTGCCCAGGAGGCTGGCTCCTCAAGCTACCCATGCCCA
GCGCCCTAAAGCAGCACCAGATGCTTTGGAATTGGGGTGAAACACCCACA
TGGCAGCCTGCTAGCAGCAGTGACTTTGACTTCTGGTCTTAAAGAGTCCC
TCACTTCAGCCCCAGGAGCTATTGGTGGGTTTTAGCAGTTTTGTCTTTAC
CGTTTTTAGTTCTCCTTGATTCTTTGTTTTCTTCCTTTATCGTTTTTAGG
TTTGGTATGTGTTGTTTTATTTCCATGGTTCCTCAAGTTTCCTTTTTAAA
CATTTGCATTTGCTGGACAATTGCAATTTTTTTTAAAAAATTCCCCTACC
CCTGTTTAAAGCTGAAAAATACATTTGGTTCATGTGCATTGTTTACAAAG
CAAAAAGAAAAAAGAGGAAAAAAAGGCAAAAAATATTGTGAAAGAAAAAA
AACAACTTAATATATTTTGGATTAATATTTGGTATTTCTTTTAAAGTATT
TTTTGTGCTGTGAACATTTTCTGCCAAAGACCATGATGTGTGTCTGTATG
TTTAAGTTATCGTAAATATTTAAAATGTAAACATGGCTGTTTTGTTATGC
CACCCTGTACCAGGATTGCTGCCGCATTCCACTGGGTATAACAGTATTTT
AATTAAAAAAAAATAATTAAAAGTGATGTCTTTGCACAGGTGAGACTTTG
CACTGAGGGAAGGGAAAGGGTGAGGATTTTGGGGGGCCACCCAAGTCATA
GTCCAATCTCTTCAAGGACCCAAGATAGAATTTAGGAATTTTGTGAAGCT
GCATTAAGGATAGAGTTGGAATGAGGATGTGGCTGGCATTTTTCCCCTTT
CATAGATAACATTTCTTGAAGGCTGGGAGGGGAAGGAGGGCCTGGCAGAT
GGCAGCCTCCCGAGGTAGGGCTGTGTTCTAATCAGCTTTGTTCGGCCACA
CGCTGTGGTTATCCACAGTGGGAGTGGGTGTCCAAAGATGGCTTTATCCT
TCCCCCACTCCTTTTCCCACTCCCACCTAGCACATAAAGTTTTGTCCGAA
ACCATCCAGAAGGCCAGGCACCATGCAGAGGACATTCAAGAATGTGCATG
TTCTTAGCCACCGCCTCCCTCCTTTGCTGTGACATCACCACTCTTTCCTG
ATGCTCAGGAAAGCATATTGCTCCAATTCAAAATTAAAGAAAGCCATTCT
CAGTCTTGCTTGTTCTTGTATCAGAAACAAAATACCTGTCTTGTGCTTCT
ATAAGTGTGTTAGGACCCTGAGGTTCCCAGTCAGCATGTGGCCAGCAGTT
AGCTGGCCATGAGGCAGCAGCAGGCTACCAGACAGCCTACGTGACATGTG
ACAGGAACAGTTTCCCACCATGGCCGTGAGTGAAGTGTCTTCGGGGCCAA
TGTGGGAACTCGGGAGCCATGTTGATGAGTAACTAAGTTGAGATCCTAGA
GAAGGTGTCTTCCCGAGCTGGCCTACCAGCCCACAAGTATGAAGACAAGC
AGGTGGAAGAATTGGAAACATCCAGAAACTTTTCTAGCAACTTTGCAGAT
GGTGATGGGTCCCACTGGATGAGGAGCTCTCCATGCAGAGGCATCAAACC
CACCCCATGGCCACACCTCCACCCAACCCTCCAGCCTGCCTCCTGCCACT
GGGGCACTATCTATGCCAGCCTTTCTCCCATTGAGCCCTGAACTAGAGTC
AGCTTCACAAATAGGTTACTCACCTGACATGCAGGCCTAGTGCATGGAGG
CGGCCTGAACAATAGGGCTCCTTCTCACCCTCCCACAGCTGCTCCTCCCT
GGGCCACAGCAGCCCATGGCTACATTTGCTGACTTGCTGTCCAAAACAAA
ATGTCGGTGCCTGTGGCAGGGAGTTCCAAAGGACAGCTTGGCAACTCAGA
CCTGTGAACAATAAGCCCTGACAACAGTGCCATCTCACCCCAGTACCTTT
GGCCAGGGGCTTCCAAAGCATCTGCACCTTCTGTGAGATCCAGTCTGTGG
AAAGTCACTTTAATGTTTCCTTGGCACAGACCAGGAGGAATAAGAAATCG
GTCCCAGTGTACCCAGCACATGTACGTGCATGTGCATTTGTGTGCACATG
TGTACATGCGTGCCTGACCATGTGTCCCAGAGAAAGGTGTGAGGGGCACC
AATATCTTCTGGCTAGTGTCCCTGCAGGGCATGGGTGCTCTCCTGCCACA
TGGTGACCAGTGCATCTGAGTGACATTTCCTCTCGGGAAAGGGGATGAGT
GAGCTGCTCGGTGCCCTGTGAAATGCAGTCTAATGAAGTTTCAGAACTAG
TGGAGCTGCAGGCTGTGGCAGGGATGGTGAGGCCTTGTGGAGGGAGGTGT
TTGCCTTCTGTGTCTAACACCTGTGAAGGGGTGGAGCCTGGGCCAGAGCC
CAGGTGGCTCAGCGGGGCCCTTTGGAGGCCAAGTTCCTCTTTGAGGGGGC
AGCCCTAACCTGCCTGCCCTGCCTCCACCCAGTGTCCCCACCAACAGGGC
TGGGGATGTGGCTCAGGGGGCAGCCCATGGCTGCAAGGGCTGGAGGGCAG
CTTATGAGTGGCTGCCCCAGCCTCTCTCCCTCCTCTCACCTCCTGGTTAA
TTGTTAACTCTTCCCCTGGCATAAGTATGCACCAGGCTCAGCAAGCTTGG
TGAAAATATGCCAAGACAAGGGCAGCAAATTGGGGCCCAGTCACCCAGAG
ATAGCCAAAGCCGCTTTACTGCACCCGAAGAACTCCAGCTAGACAGGAAA
GTTGTTTGCAGTACCACTCTGCCATCAACTAGCACAAGTAAGGGGCTGGC
CAGGATGAAGCTGTCCCTGATCAGGCCTTGCTGGGAGCGACCGACCCCTC
TGGAGGTGGTTCAGAAACCCCCGCAACCAGCAGTGCATTCCTGGACGTGC
CATGCCTGGATCTAACACATGGGTGAAGTGCCACAGCAATTGAACCCTAA
GCATCTTTTTTTTTTTTCCAAACAAGATAGAATTCACATACCATAAAATT
CACCCTTTTGAAGTGCACCATTCAGTGGTGTTTTGTATCACAGCATCTAC
TTTAAGCCAGCGGTCAACCACGGGTCATCAGCAAATGCCCCACCAGTGGG
CCATGTGGACACAATTAGAAGTGTTATTGGATCTGCTGCCTTCACACTTG
TCGGCTCCTCTTTCCTAGGCTGACACTTTGAACTTTCTATTGCAGAGAGT
CCCCTTTCACCCATCCTGGGCCCAGCAGCCCCAGAGTACCTCTGCTGGGC
TGCAGCCAGGACACTATCTGCTCTGAGCTGGAATCTCCCACATTTCATTC
CCTAGGCTGCCAGGGTGGAGGTCCTGAGGTTTGGAAAGGAGGCCCATGAG
CCCTGAGCCCAAGGCACCCATGTTTGCTAAGGTTTGCCAGAGTCAAGGAG
AATTGGCAGGGGTCGGTCCAATTTTGTGAGGCCTGAAGCCTGTATAATTT
GGAGGACCCTCTTGAAGAAAAAGAATGCAAAGTGGTGAATACAAAATTAG
ATCTAGAGTTTTGGAAGGGGCTTCTTCAAGTGAGCACCCCGAGGTACAAG
ATTCATTAGCTTCACAGTAAATCTGTCTGTCTGGGCATTGGGCATTGTGT
TTCCCTGATGGGAATGGAGCCCTGGGAAGTACCCAGGACTTGGGTTCATC
CTTGTCTCTCCCAGGTGAGGCTTGGATCTGGAAGTCAATGAAGTTGCAGT
TTTTCTTCTGCAGATGAAGTGGCTGGTACTGGATATCCACTAAAAACAGC
CCCCTCTGTAGGGTGGAGGTACCCACAAAGGGAGCCTGCGGTCTAGGCGC
AGGATGGGGTTATCACCACAGAGCCTGGATGAAAGGGCTGACCAGTTTGC
TGAGGACCAAACTGTAAATGACTCTCATGTGCACCCTGCTCTCGCCCACC
AGCCAAACCTCCAGCCCCCAGCAGGAGTGGGAGTGGGGCACATCTGACAG
TTGGCTCTCGGTGCCCTGGAGAGCCATGCCACTTGCACTTGATTGCAACA
GGGACACATTCTACTCTACTCTGTGGGCCCATCAGCTTTTGATTCTGAGC
CAACCTGGTGTCATGATCTGTCCTGAGATAAATGCCTCTTGCAAGTCCAA
AGGCTCCTACCTGGGGGCACATTCCAGGTGTGTTGGGCAATAATTTGGTT
ATTTCCCTGCACAGGTTTGCCTCTTCTTGCATCAAGTTGGCCTTCTTTTG
AAGCTTCAGGAGCCTTGAAAAGAGC

An exemplary DNA sequence of human Ryk is provided below.

gi|224589815: c133983626-133861936 Homo sapiens
chromosome 3, GRCh37.p13 Primary Assembly

(SEQ ID NO: 306)

AAGATGAACTTCAGGCAAAAGGTAAATGATTCCAGTTGGAAGTTCTGAGA
TACGAAAAGGAATGGTTGTAAGTGTAACTAAGCAAGCACTGACAAAATCA
AGTAACAATAGTAGCATTAAACTTTTGTGCATGTAGGCATAGGCTAGAAC
TAAAATTGTTGACAATAAGAGCAAATGAGTGGGGGTGGGGGTGATCATAA
AGTGTTTTAATGTTCTCACGTTCTTCAGGAGAAGGGTAAATATATGGATT
AAATTTAGACCTGGTTAAGTATGAGTTTAATATAACCAGAACAATCATTA
AAAGAACAGCCATAAAGCATACGACTTCCAAAAAAAGTAGAGACAAAAAA
GTAAAATGAGAAGGAAAATCAATCCAAAAGAAGGCAAAAATTGGTTGGGG
GCATGGGAGGAGAATAGCATAAGAAAGAAAATAGAAAATTTGTAATGAAA
TAGTTGAAATAAATCCAAATATACCTGTAGCTCCAGTAATGTAGTGAACT
AAATTCTCAAGTTAAAGACAAATATTGTCATATGTAATAAAACAAAATCC
ACACTTAACACATTGGGACACAGAAATGTTGAAGCTGACAGGATGTAAAA
TGATATACAGGCAAATGCTAACAAGAAGAAAGCTGGCAAGCTGGTATGGC
TTTATTAATTTTAGACAAAATAGACTCTAGGCAAAAAGCATCAATGGAGA
TAAAATGAGTCATACATAAGAATAAACAGCTCAAAATTTCCAGAAGACAT
ATCACTTTAAAATTTCCATGTCACTAATAATATAGTTCCAAAATACATAA
AGCTCAAACTGACGGAACTACAGGAAGAAACAGATAAATCTATTATCTTA
AGGAGAAATTTGCATACAGTTGTCACAGTAAGAGATCAGGCAAAAACAGA
AGAGTAAAAAGAAGGAACATTTGATCAACACAATAAAAAGGTTGAGGGGT
TGAAGTAATGCACATTACACAGAACTTCGCTCTCAACTATTGTAGAGCAT
ATATACTTTTCACATGAAACATTTATGAGAATTAAACGACATGCTACACC
ATTAATCTAGCTTTAACAAATTTCTTTTTTTTTTTTTTTTTTTTTTTTTT
TTTTTTTTGAGACGGAGTCTCACTCTGTCGCCCACGCTGGAGTGCGGTGG
CGCGATCTCCGCTCACTGCAAGTTCCGCCTCCCGGGTTCACGCCATTCTC
CTGCCTCAGCCTCCTGAGTAGCTGGGACTACAGGCGCCCGCCACTACGCC
CGGCTAATTTTTTTGTTTTGTTTGTTTTGTTTTGTTTTGTTTTTGAGATG
GAGTCTCGCACTGTTGCCCAGGCTGGAGTGCAGTGGCAGGATCTCGGCTC
ACTGCAAGCTCCGCCTCCTGGGTTCACGCCATTCTCCTGCCTCAGCCTCC
CGAGAAGCTGGGACTACAGGCGCCCACGACCACGCCTGGCTAATTTTTTT
GTATTTTTAGTAGAGACGGGGTTTCACTGTGTTAGCCAGGATGGTCTTGA
TCTCCTGACCTCGTGATCTGCCCGCCTCGCCTCCCAAAGTGCTGGGATTA
CAGGCGTGAGCCACCATGCCCGGCCAACTTTCTAAAGATTGATATCTGAG
CCCAAAGCTAGTATCTGTGAAGCTTTTAGAATTTTTTTTTTTTTTAAGAC
GGAGTCTTGGTCTGTCACCCAGGCTGGAGTGTACTGGCATGATCTCGGCT
CACTGCAACCTCTGCCTCCTGGGTTCAAGCGATTCTCCTGCCTCAGCCTC
CCGAGTAGCTGGGATTACAGATGCCCACCATCACGCCTGGCTAATTTTTG
TATTTTTAATAAAGACGGGTTTTCACCATGTTGGCCAGGTTGGTTTCAAA
CTCCTGACCTCAGGTGATCCGCCCGCCTCGGCCTCCCAAAGTGCTGGGAT
TACAGGCATGAGCCACCATGCCCAGCCTTAGAATTTGACCTAGAGACAAA
AACAGAACTACCATGTCTCAGAGCTGTGGAAAGTTCAGTAGTTTCAACTA
AAGAATTCCTAGAAAGGTGTACTGATCTGTTCTAAAATGAATTGTGATAG
TGGTTGCACAGCTCTATAAGTATACTAAAACTCATAGAATTGTTAACTTT
AGGTGAATTGTGGCATGCAAATTATATATCAATTAAGTTTACGTATGTAT
CAACTTGAGAAGCTAGAAAACTATGGAAACCTGTCTGTTTCTATGAGGAA
TTTTAAAAACTTTCAGAAAGAAAATATGAGCTAATGAAGCTAAAGTATTT
GGGGGAAAAAAAGAAGTCTGACTTCCAATGCCTCAAAGAAAACATTAGAC
TTAAGAGTACTGAACACTTACTATAGAAACCAGGAGACATGACGAAGTGA
CATACTGCACTCAGTGAATTAATCATGTAATAGCTGAAATCCTAAAGGCT
TAGAAGAAGAGAATGAAATTGAAATATGGACTAATTGTATTCCACAATTA
GAAGAACTGGAGGATCCCCAGAGTAAAAAAAATTAACAACTCAAAGATGA
TGGACAAACAAACAAACTCCATTAACTCTAACAGAAAGTGATTAGAAACC
ATTTCCAATGCTAACTGAATAATGAGATGTCAGCTAGGTACAAGAGGAAG
AACAATTGAAAAGTTGCAATATGAACCTCTTTGCTATATTCACCAGAAAG
TTATAATGAAAAGTGTAATGGACTGAATTGTGTCCCTCCATAATTCATTT
GTTGAAGCTCTAACCCCCAATGTGACTATGTGCTTGGAGGCAGAGCTTTT
TAGGGGAGTGATTAAGGTTAAATAAGATCAGAATAGTGGGGGCCCTAATC
CAATTGAACTTCTATTCTTAAAAGAAAAGGAAGCGACACCAGAGATTTCT
CTTTTTCTGTGGGGACACAGAGTAGAGGTCATGTGAGGGCAGAGCAAGAA
GGTGGCTGTCTACAAGCCAGGAAGAGAGGCCTCACCAGAAACCCATCTTG
GCAGCACCTTGATCTTGGACTTCTAGCCTCCAGAACTGTGAGAAAATAAA
TTTCTGTTGTTTAAGTCAGCTAGTCTATAGTCTTCTGTTACGGCAGCCTG
AGCTGACTAACACAGAAAGCAAATACAGAAGCTTCTGATTAAAGATAGTA
GAGCAAGAACATGCATTAATCTACAATCACTTCTAAAATCCCTTTAAAAC
CACCATTAAGAGATTTTTTAAAGGCATAAACAAACAAGGACAAAAAATAT
GGCAGAGGAGACAATAGCAGGAAGATGTTCAAAGCTGGAAAGCAGGACAT
GTAGTAACTTATTTTGCTGATCTGAGAAAGAGGAATTCTAAGGCAGAAAT
AGGGAAAGCAGAGAATCCATCCAAAAGACTCAGGAATTGGTGGAACCAGT
TAACTTTGCAAGTGAAAATGAAGATGGTATTGAAAATAAGAGAGTTGTTT
AAATAGATTAAAGAGCTAAAAGGTTGTTAAACCTCCAGATACCTTCCTGC
TATAGTTTGCATGTTTGTCCCCTACAAACCTCATGTTGAAATTTGACTCC
TGATGTTGCAAGGTTGGGTAGTGGCACCTGGTGAGAGGCATTTGGGTCAT
GGGGGCAGATCCCTCATGAATAGATTGATGCCCTCCCTGGGGGTGAATTC
TTTCTCTGTTAGTTCTTTCAAGTTCTGGTTGCTAAAAAGAGTCTGGCACC
TACCCCCACTTGCTTCCTCTCTTGCCATGTGACCCTTTTGCACATGCTGG
CTCCTCTTCTACTTTCCACTGTTGAAACGACTTCAGGTCCTCACCAGATG
CAGATGCCCAATCTTGAACTTTCCAGCCACCAGAATAATGAGCCAAATAA
ACCTCTTTTCTTCGTAAACTATCCAGTCTCAGATATTCTGTTACAGCAAC
ACTAAATGGACTAAGACACCTCCCCAACTCAGCACAGCAAGATGTCTCAC
ACCACCTCCCCAAATCTGGCAAAGTACTGGAGTTTTAAATATTGGAGAGG
ATAAAACTAAGGGTTAAGTTGAAGGCAAGAGTACCATATTAAAAATAGGA
AGATTTTGAGATATTCTATCTACATATTGATTTTAGGATTATGGGTCCTT
CTTTTCCCTTGGCTGCTGGAATGCTGTTAGTTGGGCCTGTACTCTTTGGT
CAAGAGATGGGGAAAATATTCCGTGGGAAATTAGACCAGCCCAAAAGATA
CGTGGAAAGATATGGACATTGGGGATTCCTAAATAAGTGGCCAGATGAGC
CACTGAGAATTTCATAGTCAACAGATTCCATCCATATCTATACAGTTTCC
AATTCAATTTTTAGTGCTCCTCTCTTCAATGAGCAACCAGGCAAGGATGA
TTTCAGGAACTGGTCTAACAATAAAGACTAAGATTAAAAACAAACAAAAG
AAAACGGAAAAATTAAAACTATACAGAGAAATAAAACTTTTTCAAAAGTT
TTTAAGAAGTGTTTTAAAAATATATTATTACTATCAGAGCTGTAAGAGAA
GATAATTATTATGAAGCAAGAATATACCATTAGGCCTGGCGCAGTGGCTT
ACGCGTGTAATTCCCGCACTTTGGGAAGCCGAGGCGAGTGGATCACGAGG
TCAAGAGATCAAGACCATCCCGGCCAACATGGTGAAACCCCGTCTCTACT
AATACAAAAATTAGCCGGGCATGGTGGCGGGCGCCTGTTGTCCCAGCTAC
TTGGGAGGCTGAGGCAGGAGAATCAATTGAACCTGGGAGGTGGAGGTTGC
AGTGAGCTGAGATTGTGCCACTGTACTCCAGCCTGGTGACAGAGCGAGAT
TCCATCTCAAAAAAAAAAAAAAAAAAAAAAATATATATATATATATATAT
ATATATATATATATATATATACACATACACACCATTAAAAAGGACATTTA
AGAGAACAAACAAGCTCTGGGAAATGAAAAATTCGAGCAGAGTTATAAGA
TAAAATTAAAGAAATTAGTTAGAAAGATTAGCAAAGAGATGCTTTAACAG
CCAGAATATAGGAATTTTAGAAAGAAGATGGAAAACAGAGTGGGGATATC
ATCAAGGACATAATTCAAGAAAACATCCCAGAACTGAATAGCCAGTGTTT
GCAGATTCAAAGGATCCACAAAATACTCTAGACAACTGATTAAAATAGAA
ACACATCAAGGTACACATTAAAAACTTTCAGAATATCAGGAATAAAGTAT
TCCAAAATTTTCCAGAGGGCAGAGGGCAAGGGGAGGAAAAGGAGAGTGGG
AGAGAAGGTTTTATACAAAGGGTCAAGAATCATAATGGCATCTGACTTTC
CAAAAGTTACACTGAAAGGCAGAAGACGATGTAAAAATACCTTTGAAATT
CTAAGGAAATGTTATTTTTAACATACAATTCTATGCTTAGTCAGTATTAT
GAATAAAGTGTTTATAAAGATGTTTGGAATAAAGACTTTCAAATATGCAA
ACTCTCAAAATACTTATCTTCCATGTATACTTTCTTAGGGAAGTGCTGGA
AGAAGTGCCCACTATATAAAGAAGGCAAATCAAGAAAAAGGACGCTATCC
AGGAAACAGGGAATCTAAAAAGAGTTACACAGGGAGGTCCCTGCATGATG
GAACATTGCAAGATGATGACTTTACAGCATCTAGGGAATAATTAATCCAA
ATGTCCAAGATGATGAAACTGATGGATCAGCTGATGTGTTTGAATACATT
GAGAGAAGTTTTAAACAACCGAGGGATGAGTTTGGGCATGAATTATTGAT
GAATACGTAGAAACCTAAGCAAGTGAACTACATGCTAACTTTAGTGGGGA
AAAGGGCTGTGCAAGAAAGGAAAAATTATTGTCACATGGCTAAGCTATGA
ATAGCATTTACACAGTCATAACTATATAAACAGTGTTTATGGTATGATGG
TAGTAAGAACAGGCAGTTATATGTGTGTGGTATTTTGGAGGGTATAAAAG
AGAGCAAAATATACATAGTTTTCTGAAGGAGCAAATCATTTTAATACATA
ATGCCCAAAATAGAAAAACCAACAGGTCGGTCATATAAACATATTATTTC
AAGTGATAGAAGTCAATAAACAGCTAAAGGGGTTTAAAGCAGCTGCCAGG
TGAGAATGGAGAACAAGTGGTAATGTTGTTTCTTGGTAATATGCCTTGTG
GAATGGTGGCTCACACCTGTAATCCAAGCACTTTGGGAGGCCGAGGCAGG
CATATCACCTGAGGTCAGGAGTTTGAGACCAGCCTGACCAACATGGTGAA
ACCCCGTCTCTTGGGGGCCGGGCCCGGTGGCTCACGCCTGTAATCCCAGC
ACTTCGGGAGGCTGAGGCAGGCGGATAGAGAGTCAGGAGATAGAGATCAT
CGTGGCTAAAACGGTGAAACCCTGTCTCTACTAAAAATACAAAAAATTAG
CCCAGCGTGTTGGCGGGCGCCTGTAGTCCCAGCTACTCGGGAGGCTGAGG
CAGGAGAATGGCGTGAACCCGGGAGGCGAAGCTTGCAGTGAGCCGATATC
GCGCCACTGCACTCCAGCCTGGGCAACAGAGCGGGACTCCATCTCAAAAA
AAAAAAAAAGAAAAAGAAACCCCCAACTCTTCTAAAAAAGCTGGGCATGG
TGATACATGCCTGTAATCCCAGCTACTTGGGAGGCTGAGGCATGAGAATC
GCTTGAACCGGAGAGGCAGAGGTTGCAGTGAGCTGAGATCATACCATTGC
ACGCCAGCCTGGGCAACAAGAGCAAAACTCCATCTCAAAAAATAAATAAA
TAAATAAATAAACCAACAACAAAACAAACTATTTGACTCTTTAAACTATA
TTTGTATATAATTTTGTTAAAAATTAACAGGCCAGGCGCGGTGGCTCATG
CCTGTAATCCCAGCACTTTGGGAGGCTGAGGCAGGTGGATCACAAGGTCA
GGAGTTCAAGACCAGCCTGGCCAAGATGGTAAAACCCTGTCTCTACTAAA
AATACAAAAATTAACCAGGCTTGGTGGCGGGCACCTGTAAACCTAGCTAC
TCAGCAGGGTGAGGCAGATAACTGCTTGAACCCAGGAGGCAGAGCTTGCA
GTGAGCTGAGATGGCGCCACTGCACTCCAGCCTGGGCGATAGAGTCAGGC
ACTGCCTCAAAAACAAACAAACAAAAACACAAACAACAACAACAACAACA
AAAAGCCCTAATTTTTTAAACAAAGAAAATGCCTGGGCCAGATGCGGTGG
CTCACGCCTGTAATCCCAGCACTTTGGGAGGCCCAGGCAGGCGGATCACG
AGGTCAGGAGATGGAGACCATCCTGGCTAACACGGTGAAACCCCGTCTCT
ACTAAAAATACAGAAAGTTAGCCGGGTGTGGTGGCAGGCACCTGTAGTCC
CAGCTACTCAGGAGGCTGAGGCAGGAGAATGGCATGAATCCGGGAGGCGG
AGCTTGCAGTAAGCCGAGATCGCGCCACTGCACTCCAGCCTGGGCGACAG
AGTGAGACCGTCTCAAAAAAAAAAAAAAACAAAAACAAAAAACAAAAACA
ACAACAACAAAAAAGGAAAATGCCTGAAACGTATTATGACATAAAACAGA
AACATTTAATGTTCTCTAAGCAGAAAGACATGACTCTGCAAATGTAGGTG
ACTTGAAACCAATATGAGAATGAAACAAAGAGAAATTATCAGTTGTGGAA
GACCTAAAAGCTTCTGAGCAGAAATTTCAAAAGTAAATAGATAAATACAC
AAAATAAATTACAGAAAAAAGAGAAATATGCTGTCACGATAGGAAGGTTG
GTGAAATCTGGCTTACTGCTTGTGTTAGAATAAGCTCTTACTTATTACCA
TGAGGACTGTAGTAATTTGTTACAGCAGCAACAAAAAACTGATGATGATG
TCGCCCTCGATCAGTTGTACCTTTTCCTCAACTGTGGAGACCTCCTCACA
ATTTAAAAGAACATCACCTAGGTTTTCTCCAGGAATAAGGTCCTGTCTTC
TGAGAGGCCAGACTAAATACATTAAATTAGGTTAAGACAAAGCCATTGTA
ACAAAGAGACCTCCAAATGGATTTAATAAGATAAAAGTTTATTTCTCTGT
CACATAAGAGTCCGGCCAGTCTCAGCTGACAGTAGGTTGTGCTCATGTGG
CCCCAGAGGCTTCTGAGCTCGTCCACCATGTTGCTCTTCTCCCTCGAAGC
ACCCTGCTTACCTCCGCAATCAAGGTGAACCATCGCCACTCACACAGAAA
GTGAGGAAGCAGAAGCTGCGCGTATCACTTTTGCTCACATTCTACTCGAA
AGAATTTAGTGAAGTACAGGGAAGGCTGTGAAACTAGCACAGCAAAAATT
CTATGAGTATGGACAAAGGAGAGAACAGATTTCAGGGGACAATTTGTGTC
CATCACCTGCATGACCCATTCTTTAGATTCTTAAGTCCCCTCAGCCCAAG
GAAGTCTCTTCACTTTGGCCCCACATTGTGCATCCCCATTACAAGGTTCT
GGTGGCCACCCTGATTCTTTTGTGAGATGCTCACTTTCTTCCAACTTAGC
AAAGATCCCTGGCTCCCTTGTAGGATAATAAAGAGGCTTCTCCAGGCTTT
CAGCCCAAGACACTGAATAAGGAATGTTTTTCATATTTCACATGCCAATA
TAAAATTTACTTTGATGATAGACATCATTTTTGCCGAATTTCTTTTTCAC
CTTGGGGAGAATGAAATTCTGCACATTTGAGCTAAACAATTCAACATTGA
GGTTTGTGGTTAAAAAAAATGCTTATGACAAGTGAAGATGCCCTATAATA
CTGTGACTTCTCATAGGATCACTTGCCTATTTTAGTTTCAGTTACTGTAG
CTAATGCCATTTGTCAATTGTTTAAAGTGCTATGAAAAATGTAATTTTTA
AAATGAAATGCTGAACAACCTGGAACTAATATCTAGATCAGCACTGCCCA
ATAGAAATGTCAGGCCATAAACATAATTTTCTAGTAAAGTCAAAAAAGGT
AAAAGAAACATATGAAATACATTTTAATAATATATTTTAACACAATATAT
CTAAAATATTACCACTCTAACATTCAACCAATATAAAAATTATCAATGAA
ATATTTTACATTCTTTTTTTTTTTAATACCAAGTCTTCAATATCTGGCAT
GTACTTTGCACTTACAGCACATCTCAATTCCTACCAGCCACATTTGAAGT
ACTTTATAGCCACATGTGGCTAGTGCCTACTATACTGGCCAGTGTAGAAC
TACATGATCTCAATGTGAAAGGTATGTGAAGAAAGAGGGAAGTGAAAGCA
GGCTGAAATTCCTGTTTTGTTTAAAGTTAGATATGGCCACTAAGGAACAT
TCAGTAAATGTAATAACTAGTGTTTGGTACAAATGGTTAACCATATGGAA
AAAATAAAATTGTATCATGTGCCATACATAAACATAAATTCCAGATAGAT
TAAGGAGCTATTTTTTAAGAGAAAAAAGTACAAAATACAAAAAGTATTAA
TTGGGGGACCTTTGAAAGACACAAAATGCGGAAGCCATAAAGGAAAAGAT
TGTCAGATTTATCTAAAATAGAAATCCAAAATTTAAGTTAAGTAAAGAAC
ATCATAAGAAAAGTTAAATGACAAGGGATACAGTAAAAGAAAACATCCAT
AAATCAATAAGAAGACAAATAAGCTAACTAAAAAATAGAATATGTGGGCA
GATATTTCAAAGAGTAATTTGAATATGCAATAAGTATATGAGATACTTAA
CCGCACTAGTGATGAGGGCAATGCAGATTAAACAACAATAAGCTATTTTT
CTTTCACTAGACTGTCAACAGCAAAATGAAAAACATTGGCCTATTACCTT
GTGTCTCTCATTTATGCCACATTTGGAAATGTATATTTGTAAAATCTTTT
GGAAGGCAATTCAGCAGAATCTATCAAAATTAAAACATAACTCCTTTGGA
CTTGAAGATTCCATTTCAGAGAATCTGTTCTTCTAAATATTTATACATAT
GCACAAAGATGTATGTGCAAGGATTTGATGTGTATTACCAGAACTGGTAA
AATCCATCAGTTCATCCCAACCAAAAGAACACCACAGGGATGCCTCTGTA
TGCCCTGTCATGAGAACATCCTCGAGATCTCAACTTACTTAGATCAGTCA
AAAAAGCAAATCCTGTAGATCACAACTTTTTGTTGTGTTGTCCATTTCTA
TAAGGCTGAAAAAGAGCACACACATACAATTGTGTGTTTAAACCCATATA
TGTGTGAGTAACTGCCTAGAGAAAGGGCTCTCAGAACATACATCAAACTG
GGCAGTGGGTAAGAGAGACGAGGGTGTGTGTGGAGGGACATATTTATATT
TTATTTCACATACTTCTGTGCTGCTTGACTCTTTTACAGTGCAAATGGTT
TAATATATTACTTGTGGAAGGAGAAGGTTTATAGAGAGAGCCACATTCCC
AACTGCTCCTGGCAAAAGACGAGGCTGATTTCAGGACTTGGAGCCACCCC
TTCTCCAACCACCCACCCCCACCCAGCTGAGTGGTTGGGTCAGGTCCATG
TTGCTGAGTTGTCTCTGGGTGTCTGTCCTTGCGGGCACCAGGTATTCTCA
GCCCACACCAGAGGTCAAACATGGAACACCCATCATCTCTGTGTAGCTTT
GACCCCACGCTCCCATCTACCCATCTACCCCATGGCATCTCAAGTCCAAG
ACTGGCTTTTTAAAGTACAAGTCCTGCTGATGGTATTTCCTGGCCATGCT
GAAATATGTCTCCTGGAGGAGGAGAATAAGGTCAACCACTGTGACAAACA
AAACCAAGAGTGGAGGGCCAATGTGTGGATTGTGTTGATGACTGTGGCTT
TCATTTCCCCAAGGCAAAGAAACCTCAGGAGGCTGTGGTCATGGTTTCTC
ATTTTCATGCTGAGGCTTTTTGGGAAATTTCAGAAGTTGCCACCCACACC
TTGTTAGGGGAGGGAGAGGAAACTTCCTCTTCAGGATGGGCTGGGGATTG
TGGGAAGACCTTTGTGTGAGACCAAGTGTCATGCAGAGACAGCAAGAGAG
AGACTGGGAATCAATTCCTTCTCCAAGGGTTTAATTAAAGTGTGGGGAAG
TTTGCAATCTTATTCAGAAGTTAACATCTGAGCATAGATTTGAAGGAGGT
AAGAGATGAAGCCATGTGGGTGTCAGACAGAAGCATTCCAGAGACAGGGA
ACAGCAGTGCAAAGGCCCTGAGGTAGGATTGTGTCTGGTTTGTTGGAAGA
ACAGAAAGGAAGCCTTTGTGGTGTGGCAGGAACTAAGGGAGTAGGCGGTG
AGCAGAAGATAAGGTCAGAGGGAAGGGAGAAAGGTGGAGAGTGAGAACTA
CCTTGACATCTTGGAAAGACTTTGCCTTTTATTCTAGGTGAGTTGGGGAA
CCACTGGTGGTCTGAAAGCCAGAGAGTACATGATCAGGCTGACGTTTAAC
AGAATGCCTCTGTCTGCTGTGTTGGGAACAGACTGAGCGAGTAAGGGAGA
AAGCAGGCAACTGCAGCTGACCAAAAGAGAGACAGGAGAGCTGAGATATT
AATGGAGTGACCAGCAGCATCAGAATTACCAGGAGGCTTGGGAAAACAGA
TTGTTGGGCCCCAGCCCTGGAGCTGCTACCTGTTAGTATGGGCAGTGTCC
AAGGAATTTACATTACAAGTTCCCAGGTGATGATACTGCTGGTCTGGGGG
CCACACTTTGAGAACTTGTCTTTGTAGAATTTATGAAAACATTTGTTTAT
CAAAGTTCTCAAGGAAGCACATTGCTCGCTCCTGTGTCCCCAGTGCCTAG
CGTGATGCCTGGCACGAGTCTTCATCGAGCTCAGGCTCTGGGCCAGGCTC
AGTGCTATTGAATGAGGTTCATGAGGGTCCCAGGTTTGAACTGTTTGGAG
GTAACTGGTGATGAAACTTTCACAGGAAGTGGTCATTAAAGGGAAAGGAT
TCCTTGTAATTACAATAACAACAGGTGTGACACCTACCCAGCGTCTGCCT
AGATGTTTTACTTGTATCCCTCTCCAATTAAATCCTGTAAAAGCCCTGGG
AGTCGGGCATTATCCCGGGATTTTTATGAATGAGAGAACCAAGGCTTGGA
GAGTTTAATTTTCCCAAGACCACAGGATTAACAAGTACCAGAACCCAGCC
AGGCCTGAGTGGGAGCTCTCTGCTGTTTACATCATAGGCCGCCTGAAGAA
GTCACTGACAAGACCTAGTAATTAAAGAACCTCGAGAAGCTAGGAGTTTC
ATTCTTTGAGGACTGTGAGCTTTTTTAAAAGTGCAAAGAATATGCCATCC
TTTAAAAAAATGCTTCCCATCTGAATTGAGATTCCTGTTCACACAACATG
TTTTGAGCCTCCAGCCCTTCCTGGCAGAACCCTGGTAGCAGTGGCTGTCC
CAACAGAAGTATTTTTGCTGACTTTTGTGGCTTAAAAGCCTTCTCTGATG
GAGCACTTTTCCTCCCCTCTTCCTCTTCTCAGCCTCCTCCGGCAGGATGA
AGGGTGTAAGATTTTGTGCTGAGGCGCTGAAGCTTGACAAATTGAGGCCA
GCCTATCTCTAAATGGAATCCGTGTATCTTGCTAGGGAGGGAGAAAATCA
AGTTCCAGCACTTCCCTCCTTATCAGCCTCTCCTTTTCCCTACCCTTCCC
TGAGGATAAAAATAACCGCTTCCCAGGGCCTCACTGCGATGGAGCAACTG
TGCCCCGAAGCCAGGCCTAGCTGTCAGAGCAGCTGCTCCCCGAGGCACGG
CCCCGCGAGAGCCTTGGGGTCAGGACGGGGGCCTGTCCGCAGGGACGAGG
TGTCCCAGGACAGGGCAGGGGCCCTTGGAGCCTCACCAAGAGAGGGGGCA
CTGGTGCAAGTTTGACCGCGATGGTGGGCGGCTCACAAAAATACATCAGG
AGGCCCGGAAGTCCATGCACTGGCTGGCTCCACCGCCTTTAAGTACACGA
GGTCTATCTGCAGTTCTTAACCTCTTTGAGGGCACAAGGACTCCCTAGAA
CAAATGATGACATACCACCTCTCTGTGTGCAATCAAGCATACACACAACA
CACAGAAACATGGACGTGGACACAAATACGCACGGAGACACATACACTTT
TGCACACAGTTTCATATTATTGGAAGGCTGTCGAAGCCTGGTATGGACCA
CAGATTAACAACCCCAAAGGCAGGTTGATTATAGTGATGCAGAACGATTA
TAAAAAAAATAACAATTTGGCAGGGCGCGGTGGCTTACACCTGTAATCCC
AGCACTTTGGGAGGCCGAGGTCAGTTGATCACGTGAGGTCAGGAGTTCGA
GACCAACCCGGCCAACATGGTGAAACCCCGTCTCTACTAAAAATACAAAA
AAATTAGTCGGGCATGGTTGCGGGCGCCTGTAATCCCAGCTACTCGGGAG
GCTGAGGCAGGAGAATGGCTTGAACCTAGGAGGCAGAGGTTGCAGTGAGC
CGAGATCGTGCCAGTGCACTCCAGCTTGGGTGACGGAGCAAGACTTCGTC
TCAAAATAAATAAATAAAAATATAAAAATAAATAAAATTTTGTAACGTAA
AAATAGGGTTAGTAAAGCGTGCTAAGATCTTTCTGGAAAACAAATATATG
TAGTATAAATTCTAATAATAGATACGGAAACGATAAAGTTGATCACCGAT
GGTAAGATAAAGGATGATTTCTATTTATTTGAGTTTTCCCACTAAACAGG
AAAGCATGCTGCTTTTTCGCAACCAGAACGGCCGCATCCAGGCGTTTCCG
GGGACCTGGGGCGCCCACTGAGCTGCAGGCGCGGCCGGTGGCTCCAGCCT
GGCCCGTCCGTCGGGAAAGGAGGGTTACAGACAATAGACAAGCAAACATT
AGACGCAGTGACCGCGTGGGGCGGGGACCGGCTCTTGCGGAGAGGGTGAC
CCGAGCCCTTCGCCCGCCGAGGCACTTTAAAACCCCAGCGCGTTGCCCAG
GCCGGCCCGCCCAGAGCGAAAGGTGCGTGCGCGCTCCCGGGGAGGCGGCG
GCGCGCACGGGCGCGTCCCGCGGGCAGCCCCCAACTCCGGACGATGCAGC
GAGGAGGCGGCGGCGGCGGCGCGCTCCGGAGGTCTGAAGCCACTGCTGGC
TGCCACGGGCCGGCGGTGCGATGAGCTGGGCAGCCGCCCCCGGCTCGGGG
CTGTGAGCGGCTCGGGGCCGGGGGTGGGCGGCGGTGCGGCGGGCGGCCGA
CGCTCCTCTTCGGCGGCGGCGGCGGCGGCCATGCGTGGGCGGCGCGGCTG
GGGCGGCCGGGCCGGAGTTGCCTCCCGGGGGCCCGCGGCTGAGGGCCCCG
CCGCCGCCGCCGCTGCTGCTTCTGCTTGCGCTGTTGCCGCTGCTGCCCGC
GCCTGGCGCTGCCGCCGCCCCCGCCCCGCGGCCCCCGGAGCTGCAGTCGG
CTTCCGCGGGGCCCAGCGTGAGTCTCTACCTGAGCGAGGACGAGGTGCGC
CGGCTGATCGGTGAGTGGGGCCGCCGAGGCCGGGGCGGGCAGGTCGGGCC
GGCTGGGGCAGCTTCCGGCCGCCGGCGGATCAGTCTGCGAGCCGTGGACC
TCGGGCGAGTACCCTCCCCGCCCCCCGCGCCCGGATTCGCCTCGCGGCCC
GGTCTCTGCCTGCCGTGCTGGCTGGGAGGTGTGGTCCCTGCCCGGTCCCC
AGCTCTGGGTCAGCCTTCGTGCCGCCCTCGCTGCCCCGCCGCGATAGACC
CAGATTCACCCCATCCCATCCTCCTCACTGCCTCCTTAGCCCTTCTCCCT
AGAAGAGTCAGTCACCCTTACAACTTTTTTTCCTTGCTGCACGAAGTTTT
TTTGACGCTTTCTATGTGTGTCACTCTAGGGCCCTCGATTTTGAATATCT
CGAAATTCACGTTTTAGACTGAACTTGCAAACCCTTTGCTGTGGTTTTGA
TGCTCCAGTAGAGGCCACGCTCTGTGCTTACCCTTTTACAAAAAAAAAAA
AAAAAAAAAAAAAAACGAGAGAGAAAGAAGGGGAGGTTATAAGGGCACCA
AATGTTCGTGGCAGCCTCCAACTCTTGGGACGTAGATTTGGAGCATATGC
TTAGATTCTAAGCATATTTTTAGTGAATGAATCGCCAGGTAGCAGACCTT
TTTAGTTTCAATACCTTTTTGGTTTTTGCATTGGGTGTGTTGGGAGTTTG
GACTGACTCTGTACTTCTGGGCCACTCTCCTACTTTTGAGTATCTTGAAG
GGAAACAAGAACTTAGTCTTGGGTCGGTTTGTGGTTCCATTTACTTGCTT
AGCTTTGTTGTTTGGGGAATTAATTTGAAAAATTGCTGGGGTCTTATGTT
AGGACTTTGTGGATTCTGGAAATGACCCCTTAGAGGAAGATCTGACAGGA
ACGACCCCCCAAAAAGGAAGTCCTTGGTACCTAGACGTTTGCATTCTTTT
GTTATTATGGCAGTATCTTTATTTCATCTGAAGGTGGTGCTTCTGAGTGT
TTGAGGGGAAAAATTATTCTCTATTGCATCCATTATTTGTCAGTATTCTA
TTTTTTTTTAAGTAATAAGACTTTGTTGAGGTGTGTTGAGATTGACTCAG
TGTAGAAATGGAATTACTGAAAGCAGACTCTTTAGACATTACATGTACGG
ATTTGCAGTTTTTAGCCAAAGAAGAATGCTCTTTTTATGAAGTCGTGCTC
ATTCTTAATCCTTCTTAGTCTAGCTGAGTTAGCTGAATGTAGTTTAGCTG
AATTAAAACTCTACTTGGGGTAGTACTTGGAACACAAGACTAAGATTTAG
GAAAAATAATACAAGACGACTCATATCACATTAATTTTAGAAATATTAAC
GTATTATGCATGGTTTTGTAAAGGCTTTTTGGTTAAAAGCACTTATATAG
CTTGTGACTTTGTTTACATGGGTAAACAAAGTCAGAGGTGTAGGTAGGAG
ACAGAAGACAACCAGGGACTGACATTGAATTCCTGCCATATACCTGCCCC
TACATGCTTTATACACATAATTTCTTCTAATTTTCTCGGGGCCCAGTGAG
GTAGGTGGCATTATGCCTATTGTAGAGATGAGGAAAGTGGGGTTTTAAAG
TAACTTGCTCAAGGTCACACAGTTAATTTTTTTTTAGACGGAGTCTCGCT
CTTGTCACCCAGGCTGGAGTGCAGTGGCGTGATCTCGGCTCACTGCAATC
TCTGCTTCCTGGGTTCAGGTGATTCACCTGCCTCAGCCTCCCAAGTAGCG
ACCACTTCTGGCTATTTTTTGTATTTTTAGTAGAGACAGGGTTTCACCAT
GTTGGTCAGGCTGGTCTTGAACTCCTGAGCTCGTGATCTGCCCGCCTCGG
CCTCCCAAAGTGCTGGGATTACAGGTGTGAGCCACCGCACCTGGCCTAGT
TCATATTTTTGAAAAAAATGAAACAGTGTTGACATAGGCTGCAAAGTTTT
ATCGAATGAAATGAAGATTTTGTCTGTAGAAGTTTGATTATGTCATGGTG
GAAAATAAATCATTGCTAATTAGCACTTGAGTTATATATTTGTTACCTTA
TTCAAAGGTTGGCCTGGAACTAATTTTCTTAGCAGTATTTGCAGCAGAGG
AAGCTCCTGTTTGATGAGCATCCAATTTATGCCCAGAAATGCTGTTGCTG
GGAGGGTAGGAAGCATTGTTTCAATTAACTCCCTTGTCTATCCCCGGGGG
CGAGAGAGACTTCTAGTAGTATTTTCTCTAGTGCAAATAGGGTTGACCTG
TTACAGAGGATCTCTAGTCACTCCATCCTGCCAGATTCAGGTTCGTTGTT
TCCATCCCTTCCCATCTCCCTCATACATTCTACCTTTAAAATAATTCCTT
GGCTCCTCCAGGTAGGGAAGAGCCAAGTGGGTAGGACTGAGATGTGTACT
GAGATGTGTACAAGGCACTCCGAATTCTCAGAATGCAGTCTCTCACAGAA
GCTGTTATGGTACTTAAGGCTTCTTGGAATAGTAGCTTTGCACCGTATTT
TAGAATCACTACAGATTTTGTGCCTTCTGAACGCTGGCATAGGAAACGAA
CCATTATTTTATAATATTTTTAAGACAGTTTAGAGTTCCAAGTAAGAAAC
TTATAAATGAAGTTGATAGTGACAACTTTATAAATTGAACTAATTACCCT
TGATATGAGAATAAATTGTTTTAATCCAGTTAATATATGCATATAGTTTT
AAGAGAACCAAAAGGCTTTTTTTTTTTTTTGAGACGGAGTTTCGCTCTTG
TTGCCCAGGCTGGAGTCCAATGGTATGATCTTAGCTCACCGCAACCTCTG
CCTCCAAGGTTCAAGCGATTCTCCTGCCTCAGCCTCCCCAGTAGTTGGGA
TTACAGGCACGCATTACCATGCCCAGCTAATTTTGTATTTTTAGTAGAGA
CGAGGTTTCTCCATGTTGGTCAGGCTGTTCTCAAACCCCCAACCTCAGGT
GATCACCCACCTCAGCCTCCCAAAGTGCTGGGATTACAGGTGTGAGCCAC
CGCATCCGGCTTAAAAGACTTTTGACAAACACAGCAGGCTCTTGCTTTTG
TCTTTTTTATCCCTCAGGCTCTACTTTCCAGGGGTAGCTACTACCTCAGC
TAGGTCTGTTTATTTTTTCTTCCTCTTTTTTTACTTTCGTCATTCTAAAT
CAATGTAGAATAAGTCTTTGACTTTCTAGTATCATAACTGAGCTCTTTCT
CATTTCTCTCTTAAATCCTCCAATGTAGTTACGTAACTTTTTTGTAAAAA
TAGCAGTATTTACATTATTGTGTACACAGGTATTGCTAAATAGTGACTTA
TGATTTTTTTTATACATTTTTGTTTTTCCCACAGCAAATAATTGCATTTT
ATCAAAATTGCGTTTTGATTCGTTACCTATTTATCTTTGGCTGTGTTCCC
TCAACTACTCCAAGAAGTATGTTAAATGCCTTTTGATTTTTTTTTTTTCC
AGGTAGCACATTAAATCATCTATCAGTGCCACTTGGTTTCCTCTTAGAGA
CTTCTTGGTCTGGAGCATTCCATCCTCTTGCTCCAATCTAGATAGTACGA
TTATTTTTTCTGGGACTTCCTGTTACTTCTTTCCTGTCTTGGAACCTCTG
TTTTTTAGATCTCCTGTCTTCCTTTTTCTTGGTTTGCTTCATTTTGCTAC
TATGTATGTATCCTCAGTAGCTTCTTAGGAAGGAATGATGGGAGATAAAT
TACTTGAGGCCTTGAATGTCTGAATGTATCTATCCTGTCCTCATACTTTA
TAGATGGTTTGGCTGAATTTAAAATATTAAGTAGAAATACATTTTTTCAT
AATTTTGATGGCATATTCACAGTTTTCTGTCTTAAGGGTTTTTGATGAAA
AGTCCAAAGCCATTCTGATGCCTCTTTTTTTCTGTTTTTTTGAAGCTCAG
GTTCTTTTTCTCTGGCATTCTGAAGTTTTATAAGTTTTTGTGTGGTTGGT
TGGTTGGTTCTTTCCTTCCTTTCTTCCTTTTCTTTCTTGTATTACCTTAG
TGGGTCCTTTCAATCTAGAGACTCTTTTTAGTTTTCAGAAATGCTTTTTT
TTTTTTTAATAATTCCCACCCTTTATTTTCTCTCTCTCTGTGTGTGTGTG
TGTGTGTGTGTGTGTGTGTGTGTGTGTGTATTTCTTTCTGAGATGTCTGT
TGGTTTGGTTGTTGAACCCACTACAAATGATCTTTAAATTTTCTTAACTA
ATCTCATGTCTTACGTATTTCTTTGTACTGGAAGGAGTGGGAGGGGGGGA
ATGGTTCTCAATTTTAATGATCATGTTTTTAAATTTTAGAATCTTTTTCT
GTTTCCATTTCATTGCATCTTATTTTATGGATGTATTATCTTTACTTATC
TCTGATGTGTGTATTTTTATTTTTTCTTTGTGTTTAATATATTTTTAATA
TTTCTTCTGTTTCCTCCAGTGTGGTTATTCATTTCAGGGATCTTTTCTTT
GTTTGTTTAAGTCATAAATATCTGGTGATCCTTGACTGTCTATAATGAAG
AAAAAGATCCTGAGGAGCTGATTGCAGAGGAGAGAACATGTGTGTATTGG
TGGTCTAGTGACTTGTGAGTTCAGGTGGACTTTTCATTGGGAGTCTGCCC
CGAATATCAACACCTTTACATCTCGTTTGGGGCCAGTGTTCACAATGAAG
AAATCATCATCTCCTGCCTCTGGTATGCTTGGTGATATGCCAGCTGCCAG
GGTTTGGGTTACCTGGCTGAGGGAGGGATGGAGGCTGTAGCCTTCGACTT
CTGGCTACAGTTCTCCTGTCACTGTGATTCAGTTTCTTCAGAAAAAACTT
TGGGTTTCCTGCTGGAACTGGGGATCCAAAAGCTTTGAACACCTCTTTCG
GTGGCTCCCCCCTTGTCTAGGCTAAGCCTCACATCAGCCTTTGGCATCTC
CTTGGTGCCTCAAGACCTGAGCCTCTTGGAGGTCCTGAGGGGCAGTGACC
ATTCCTTCAAGTACTTTGCCATCTTGCAAAAGTTTGTTTAAAAAAGCATG
TTGTCTTTCCTTTTCTGTTTGTCCTTATGGTTTGATGTCTTTGTGATTTT
TTTTTTTTAAACTATCATTTTAGAGGAATTTTGGAAGGAAATGGAGGTAA
GGGATTATGGTCATTCCACAATGTGTAAATGGCAGTTAGGGTTTGGGTTT
AACTATTGAAGATGACGTGTGGCAGTCTTTTTATATGAACAATATTACGA
GTAGTTAATGGTGGTTGTAGTAGTAGTTGACACTTTTAAAAGTGCTGTCT
TTTGTCTGTGTGGGGCATAGAGGTCATCCCTGTGTTATGTACTGTCATTT
CCATTTTATAGATGAGTGTTAGTCTGCTCAGGCTGCTGTAACAAAGTACC
ATAAATTGGGTGGATTGTAAACAACAGAGATTTATTTCTCACAGTTTTGG
AGGCTGGGAAGTCCAGGATCAAGGCTTTGGTAGATTTGATATCTGCTGAG
AGCCCTCTTCCTGGTTTATGGATACTGCTTTCTCCCTGTGTTCTCACATG
GTGGAAGGGGCAAACGAGCTCCCCTGGGTCTTTTTATAAGGGCTCTAATC
CCATTCATGAGGACTGCTCTTATGAGCTAATCACCTCCCAAAGGGCCCTA
CCTGCTAATACCATCCCTTAGTGGTTAGGATTTCAACATAGAAATTTTGG
GGTGACACAGAGAAATGAGAAAACAATAATCTTAGAGTAGGTATGTATTA
ATGACTAGCCTGGTTTTTTGGTAGTAGTAGGGCACACGGTCCTGATGGAA
GTGTATAAGTTCTGAAACACCGAAAGCTTCAGAAGCTTTTAAACTCTGGT
TGGCCTTCCTGTGTTCCTGGGCCCTGGTTCATCGCTTGCATTTACAAGCC
TGTTACTTGCATAACAGGCTGCAATGTAGGCATTCATTTCTGCATTTTAC
TGATGAGGACCCTGATTCAGGGCCTTTTGGACAGTGAGTAGAAGGCCAGG
TCTTGACTCAGTCCTGCCTGAGACCTGAATTCATACTGTCTCTTTATCAG
CTCTGGAGGAGTGCAATTAACCAAAAACTCAAAGAGTAGAACCGCAAGCC
TCCTGCCACAGGTGCTTGTTTGTGGTCTTTATTATTATGCTAATTATTCT
TGCATCCTCCCATGTTCTTTAATTGATAATTTTCATTGTACTGAGCTCTG
TGGAATTCCGGAGCCTGTGAAGGTGGCATATGGCTGGGCAGTGCCAGAAG
CTTGGGAAGAACATGCTGGTGGGTGGGTGGGGAATGGTATAAACTGGTTA
TGCCAACAGTGGTGGTGGTGATAGGGGTCAGGGGGACTTTTGGGAAGAGG
CCCCATCTCCCACCAGTTAGAAATTAACTTTGGATAACTTGCTTCTCCTC
TTAAAATAAATATCTTCTGGAAAAGTCAAGAATGTTCACATTTTATTTAA
AATCAAGTCAATACTAGTTGAAATCACTGTGTCCCAAATCAGTCCAGAAA
ATTTTTTTGAAGCTAAACCAAGTACCATATATTGTGGTAGAAAATGACAA
TTCAAAATTATTACACTCCTTTCCCTGAAGATCTCAGAATTTTGTAGGGG
AGACTTGCATTTATGACTGTAACAGAGATAAGAGTTATATTGCTGGTTGA
CATAAGTTATTGTAGGAGCACTTACTTTTGACTGTGGAAAGGGCGGAGGG
GAGACAGGCTTAGAGAGTTTAGGAAACTTGTCCCAGGGAAAAAAAAGACA
TTTGAGCCTCATCTTGAAGGATGAATTTGATAGGCAGAAAAGAGCAGTTC
ATACCAGGCTGAGAAAGTAGTGTGAACAAAGACATAGAGGGTGGAAGCAG
CTGTGTGAAATTGTGGGGTAGGAAAAATCTAATGTGGCTGAGGTGAAAAA
TTAGCAAAGCCATGGAGCGCCACAGGATAAAGCTGGAAGGTAGGCTGAGG
TTAGAGTAGGAATTTGAACTCTTACAGGTGAATTCTTCAGTGAGCTGCTA
AAGAATGCAGACTAGGAGAGTGCGCAGTCAGGTCTGGGTTATAGGGAAGA
TAACTTTGGCCCCAGTGAAAAGGATGGCTTTGGAGAATGAAATGCTGGAT
CTGGGAAATGAGATAGAGCCAACCTTGTGATGATAAGAGGGATCAACTAA
GGCAGTTGAAGGGGTGGGATGGATTTGTGAGGAATTTGGTGAGTTTGGAG
AAGACTTAGTAGAAGAGACTTTAGGGCTAAAGAAAGGGGAGGAGTCACAG
AGGAATCTAGGGTTTCAGATTTGGGCAACTGAACCAAAGGACCCTTTCCT
TTTGGGAGAAGTGGTTGGTCAAGGGAAAGAAAAAGGAGATGAACCATGTA
TTCCTCTCCTCCCAGCACCCCTCTCTCCTAAATCTCAGAGCCCATGCTTG
GTGGGGAGGAGGGGTGGAGGGTTGAAGGTGTCCTCTGCCCATGAGTCTGG
CATAACCTGCCAGAGGGCCCAGATCCTGGGTGGGTTTTTGGGAGGTTAGC
ACTCTGCAGCAAATCCTTAACCCTAGGCGTTTTGGTGGAGTTGGTATTTA
TTTGAATCCATGTGTTCTTCATTGGGAGACAGGCAGAGACATTGTGCAAT
TGTGGGTTTGAGGGTTGTTTTTTTTCCTCATGCTACACATGTGAGAGAAC
TCTATCCAATTATGGCTAAACCTGTCCCCTGTGAGTCACATCCTGAGTTG
ACTGGTCAATTCCAAAGGTGCTGCCTAAAATGCTGTTTGGATTTTAAGAA
TCACTTTTGGGACAAATCAAACATGTGAGTATTTATTCAGGCACCTGAGT
TGAGAAGTTCCATAGCATTACTTTTGGTGTTTTGATGCAGTTACGCCAAT
GTTCTGAAAAATCTTGCCAGCTACACATCCTAAAGCAAAACAAAAACTTG
TACATGAAACAGAACCATTAGCTACTCTAAAATTATGTCACACTTAAAGG
TATACATTTTTGTGCGTTGTTGATGTAAATCAAGTGCAATATTTTCTAAT
AAAAATTTTATTAAACAGCATTCATTTGTGAAGGTCCTCTTAAAATGTGC
CAAAATAAACGTGTGTGTATTGCTCAGTTACATAATAAACAGTTGTTGAA
TGATTCTTTTTGTGTGTTCAGCAGTGATAATGCTGAGTGGATTTCATAGG
TCTCATTCTTATCTTTTTTTTTTTTTTGAGACGGAGTTTTGCTCTTGTCA
CCCAGGCTGGAGTGTAATGGCATGATCTCGGCTCACTGCAACCTCTGCCT
CCTGGGTTCAAGCAATTCTCCTGCCTCAGCCTCCTGAGTAGCTGGGATCA
CAGGCGTCCACCACCATTCCCAGCTAATTTTTGTATTTTTGGTAGAGATG
GGATTTCAGCATGTTGGCCAGTCCAATCTTGAACTCCTGACCTCAGGTGA
TCCACCCACCTTGGCCTCCTTTCAAAGTGCTGGGATTACAGGCGTGAGCC
ACCTTGCCCGGCCTCATTCTCATTCTTATATAAATTTTAAGCTGGAATAG
ACAGCACACAAGTCTCTAGTTAGGGATCACCTGTAATTTAGGGAGGAGGA
GGAAGAGCGTATCTTGCTGTATGCCAGGTACTTTACATATGTTCTCTTTT
ATCAACCATAGGAAGTCGGTGGCATTGACTGTTTTTTGTTTGTTTGTTTT
TAAACAGATAAAGCTGAGATGCTGAGGTTGTGGGCAATTAACAGAATTTG
CTGTAGGTCACAGAGATGTGTGGGGCTATGTGACTCCAAAGTGCGTGCTT
TCTCTACTGTGCTGCCTTTGAAGGTTGACAGTTGACTTAGTCTTTAACCA
AAATCATCTGAGAAGCTTTAACAAACTACAGATTCCTTGACCTACCCTGA
GCAATTTGTTGAATTGATCTGAGATGGGGCTCAGGTAAATTAACGTTTTG
AATGCCATGTTTAATTAATTACAGGAGGTACAGGTTTTCCACATTTGAAC
GTCTCTGAAATCAGAACCATGTTATAATCAGTGGGGAGAGTCATCAGTTA
ATAGCTTTTTTCTTTCTTAGTGGCACAAAAAATAAGGATGTACTTTAGAT
TAAATGAAATATGATATTATAAAAAGGTGATACTGTAAAGGGAACTTAAA
TACATAGTTTGTCACCTTTCTTTTTTTTTTTTTGAGATAGAGTCTTGCTC
TTTCTCTCAGGCTGGAGTACAGTGGTGCAATCTCAGCTCACTGCAGTCTC
TGTCTCCCAGCTTCAAGCGATTGTCGTGCCTAAGGCTCCTGAGTAGCTTG
GGACTACAGGCGTGCACCACCACGCGTGGCTAATTTTTGTATCTTTTTTT
TTTTTTTTTTGGTAGAGATGGCGTTTCACCATGTTGGCCAGACTGGTCTC
GAACTCCTGACTTCAAGTGATTTCCCTCCCCCACCAACCCTGTCTGGGCT
TCCCAAAGTGCTGGGATTACAGGTGTGAGCCACCATACCAGGCCTCCACC
TTTTTTATGTGCATATGCATGTGTTTGTCACAACTTTCAGAATTGTAAAA
TTCACTGGTGGTATTTATTATTTGCTTACAGCAATAGCTGTGTAGTAATG
CCCTTGCAGGGAAATCATAAAATGAATGAGATATTGTCATTAGATTAAGA
GCAATAGCAAGGTTACTTTTCAGCCAAGAAGCTGCAGGCAAGGAGAAATG
TCAGTGGTTCTCAGCCTTTTTTTGTGAAAGCCTTAGGCCCTTTCCCTAGA
AAAAGGCATGAATGTCCACACATACTAAAATTTGTATACTGTGCTAGGAC
CTCTAGACTTTTGGGGCCTTTGAGGGACTCCTGATGAAAAGCTTAATGGG
TACACCTGAGTGTCACAAAAACTGGTATATGTCATATTCTGTTTTACATC
CTAAGATCTGACCTCCTACATGAATTTTTTTGAAAATTTGCATATATTAA
GATTCACTCTGTTGAAAAGTTATGTAGGTTTCGACAAATGCATAGTGTCC
TATATCCACTACAGTATCATAGACAGTAGTTTGCACCTTTAAAAAGTCCC
CTGTGAATGGGGCATTTGTATTTATAAATACTCCCTCCCTTATATTTTAA
ATAAGGTGCTTTAAAAATGTATTTATTATGGAACATTTCAAAAATTCAAA
ATAAGGCATAATAATATAATGAATCTCTGTATACCCGTCATCCAGCTTCA
GCAGTTCTCAACTCATAGCCAATTCTGTTTCATCTCTACCACCTTACCCC
TACTCCACTCATTTGCCCTCACTCTCCTTATCCTCTACTTAGATTATTTT
GAAGCAAATCTAGACATAATTTCATAAAAATATTCCAATATGTATATCTG
AAAGAAATTCTTAAAAAATCTAAAATAACCATTATTGCACATTATTAAGA
AATTAAGAATTTATAAATATCAAATATCTAAGCTGTTGAGCTTTTCCTGA
TTTATAAATATGTAAATGTATATGTAGAGACTCTTCTTGCCTACGACCTG
GATCCACTCTTATCCCTGCCCCTCCCTGCACCACTCAGTTCCCTTGGTGC
CAGACTCCTTCTCACCCTGGAGTCTACATTAGATGGCACCTCTGCCTTGC
GTTCTTTCCCCAAATCTTTACATGGCTGGTACTTTCTAGATCTCAACTTA
AAATGTCAATTCCCCAGAGAGGCTCATCTTCCTAGGGTTGATCCCCAGTG
CCTCTCTGTGCTATCACACTGTTTTAAGCATCTGATACTTTTTTGGTTGT
TTCCATCTCCTTTAGTGAAAGTTCTGTTTGAATAGACACTTTTTAGGTTG
TTCCACTTTCTATTTTTAGCACCTAAAACAGTATCTGACATGTAGTAGGT
TTTCATTAAAAGTGGAACAAATGAGTAAACAAGTGAATCATACTTACAAA
AAAATTCACTATCCTGTGCCTTACTGGAGTCATCATTCTGTTGATCATTT
CCCATGGCCTGCTTAGTTATTATCCAAAAGGGAAATTAGACTGAAGCTAT
ACTTACTAATGGAAGTTTACATATGTTAATTTTTTATAGCTGCTTTTCTT
TTCTTTTTTTTCCCCCTTTTTGAGACAGGGTCTCTCTTTGTTGCCCAGGC
TGGAGTGTAGTGATGCAGTTACGGCTCACTGCAGCTTTGATCTCCCAGAC
TCAAGTGATCCTTCTGTCTCAGCCTCTAAATAGCTGGGACTACAGGTGTG
AGCTACCATACCCCACTAATATAAAAATTTTTTTTTGTATAGATGAGGTC
TCCCTGTGCTGCCCAGGATGGTCTGAAACTTCTGGGCTCAAGGGATCCTC
CCACCTTGGCCTCCCAAAGTGCTGGGTTTACAGGTGTGAGCCACTATGCC
TGGTCTTGATAACTTATTTTCTTTGGAGTTGCCTAATTTGCCTCTCCCAT
TCCCTTGCTGTCTCTGGGTAAGGCGCCTCCCATGTCGACTTCCATAGCTC
CATACCCTGCATAAGCAGACCTGTATCACTGCACCCATCATATTTTGTCT
CTCCCCTACTGCACCGAGGCTACCTTATGGCTTGGCACAGAGCAGTCTCT
CAATAGACTGTTGGCCCACGCTACAATTGGAGGATGTGAAGTATTCAAGA
GAGATTCCGGCAAAAGCCTTTATCAGTAATGAAAAGCTAGGAAATACAAT
TTCTAATTTTTTTTCCCCAAAATGCATAACCTGCTGTCCTATCCAGTCTA
TTTTCCATCTTATATACAGTATAGTTTTCAGCCCTAGCAATTTGACTTGG
GTCTTTTTGTATTTTCCATGATTCTACTTAACTTTTTGACCATGTGGAGT
ACAGGTATAATAGTTGTTTCAGTGTCCTTGCTTGTATTCTAACATTTGTG
CCTGTTCTGTGTTGATTTTGACGACATATCATGTTTACTTCCTGAAGTGG
CACAGTTAGGAAAAGTTAGTGCCTGCAGATTCCTGGCTTTTCCTTGGGAG
GAACAGGAGATTCCCTTCCATGTCTTCTTGAAGCTGATTTCTAAACCACC
TATCAGGACCAAAAGGGTAGGCTTACCTGCTTTTCTTTAGGCAGAAAGAA
GGCAGTCTGAAGGACGGAGGGGAAGAAGGGGTATATTTATGTTGTCTGCT
GTTGTTGGTCATGCATACGTTCTTTCTCTGGAAAGCTCTCTCTAATCCTC
CTTTCTGTGTATGAACATACACTCTCAAGACAGTTCTATCAAGACACTTA
GGGTTGCAGGACAAATATATGAGGAAATGCTGAAGAATGTGGCAGAGAAT
TACACGAAAAATCATATTTAATCCACAGATTATTGCTTGAACAGCTATGG
GATTGAAGATTTACATGCTTATTCAACTATGGGGAAAGGATGCCCCTGTT
CTACAGTCTAATATTTTAATTCCTTGTCTTATAATGTAGTTAAACATTAG
GTCTCAGCTAATTTTCTAGGCATAATATGACCACAGCTAGATTTATTACA
TTCTGATGAACAAACTGTTATTTTATCGATTTCAGCTTGTTACTTTCCAT
AACCTAAGAGCATTTGAAATAAGAGCTCCTTGTTTACTTTCCTTTTAGCA
TTCATTAAAATGTATTCAGTGGTTTAATACATGTTAAGTGATATTGGTTT
TTTCTCTGATTAATGGCTCATATTACCACTGTCCTCAGAATCTAGAAGAT
TATCTTGACTAAATGAATTTTTACTTAAGGTTTAAATGGCATATGGAAAC
TGTGGATGATATAATAAAATAATAGTGTTTTGGCTGACACTAGGAAACCA
TGGATGATATCATAGAGTAATAGAATTTTGCGGCTGAGGAGAGGGACATT
AACATCATCTGGTTTAACTTTTTTGTTTTGTAGTTGAGAAAATTGTTGTC
TAGAGAGGGCGGTTAAGTAATTGGTTGCCAAAGGTCATTCAGCTACCTAC
TGGTGAAGCCAGGGGAAGGGAAGTAAGAACTGTGAACTCTGCTTTAGTTC
TCTTTCTATGACATCACTTTTGTTTTTTTGCCTTAACTAATGGTTCTTGA
CATTTTCTTTAAAGTATCATTTTCTTTAAAACCCATTTTCTTTAAAACCC
ATCTTTGATGTAGTTGACTTTTATCGACAGCCAGCCATTCATGCATCTAA
TATTTAAGTAGTTACAAGTAAACTAAGACCCCAGCCTTGTTTGTGAGATG
TGCAAGGTAATAGGAAGATGGACATTTGAAACAACTCCCTGAAATATGTT
TTGTTAAGTGCTATAAAGGTGGCTTTTAAAATAAAGAGAGATGGTTTGCT
GATTTAGCACGAATGAACCATATGTACAGCTTTGAGAAACCAATAGTCAA
CTTTTAAAATCATTTGGAAAAAATTTTTGAACTGTTGAATTATTAGAACT
TGAGTTAGGCCAGTTCATGTGGATCACAGACCTTGAAATAAATTTGAACT
TAAAATCGTCTAGGGAAAAATGAATTTTTTTTGATAAGCCTTAAGAGTTT
TGCAAGAATAACTTTGGTTTTTTAGCTTTGAATTTGAGTTGTTTAAAAAT
AAAATTTTGAAAAAATGCCATGCTTTCTAACAAGAGAGCAACATTTTTCC
TTTGCTGGAATTGGCAATGGGAAATTTAAGTCTGGTTAATGTAACTGAAC
AGCTGAAGTATTTTCATCCTAAATCAGAATTTAAAAATTCTGTGCATTCT
AATGACAAAATGCTACCTCTTTAGTTCGTGGCTGTCCATTCTTGTTCTTG
TCTGCTTTCCAGCACAGCCCCCTTGTCTCTTGGCATCCTTGTCAAAAATT
AATCAGCCCAAAATATGTAACTTTCTTTCTGGACTCTCAGTTCTCTTCTG
TTGGTCTACATGTCTGTCATGCCAGTACTACACTGTTTTGATTAAAGTAG
CTTCGTGGTGATCTTTGAAACCAGGAAGTGTGAGTCCTCCAACTATGTTC
TTTTTAAAAATTGTTTTGGCTATTTTGGCCTGCTCACAATTTCATATTAA
TTTGAGGATCTGCTTTTACTCTTGTGCAAAAATGGGAGTCAGAAGTTTGA
TAGGGATTTTAGTGAATCTGTAGCTCACTTGGGTAGTATTGTTATATTAA
CAGTGTTAAGTCTTCCAGTCCATGAACATGGGTGTTTTTCCATTTATTTA
GGTCTTTAATTCTTTCAGCAATGTTTGTAGTTTTCAGTGTACAGTTATTC
CTAAGTATTTTATTCTTTTGGATGCTATTGTAAATGGAATTGTTTTCTAA
TTTCCTTTTTGGATTGTTCATTGAGGGACCAGCTCTTATTATGCATTATA
CCTCTCCTTTCCTACTTGGATTGTTTTGCCCACTACAGTGTCTCAGCATT
CTCTTTTCTTTTGATTTCTATGATCTTTACTTACATAACCTTTGACTCAT
TAGGATTGTTGGAGTCACCAGACCTCTACAAAATGATACTCAGTGACTTT
ATTTCCCTGACTCTAATTTTCATTTATTCCCTCCCTTTATCCCAAAGGGA
TATTAGAACCTACCCTGATTTTTTTTTTCCTTTGACCATGCGGGTGTCCC
AGTTCCCAGTTTTCTTTTTTTTCCCAAATAAGGCACTCATTGGAATGACC
ACCTTAGAAGTGATCTGTCTCAGCCCTTGGAATTTCCTAACTATAGCACA
CCTACCTTCCTTCTCAGTCACTCCCTTCTATTGGAACCAGGCTCTGTTTC
CTAGTCTCATAAAGAGTTACCCTGCTCCTGGCTTGCCACAGAACTGGTTC
CTTAAAGCATTACTGAATAGATTGTTTCCCTCACTGCCTCTCTAATCATT
TCTCACTACTCACTTAACCTCTGTGATGCTTAACACTCCCTGACAGTAAT
AACCCCTGCTGTCCACTGTCCCTGCTTTCAGACTGCATTTGATTGCTGTT
GTCGTATTTGAAACACTAATAATTAATAGTAATTAGAAACAACTAGGTTA
TCTAGCCCCTGTCTTTATGTGTCTTTTGCTTTTCTCTTTTAATTTTTTTC
AAGCGTATGATGGGGAAGGAAAGAAAAAGGAGTTAAAATTGAGAGATAAA
GTATGATATTCTCTATAGATTTCATCTTTTTCTAAAAGTAAATTTGTTTA
AAATTCTTTTATTTCATTAATTTTATTTCCTGATTATTAGGGTAAAATAT
ACTCATTTATAGACTATTTGGAAAATATACAAGAAAAGATATAGACTGGA
AAATATACAGGAAAAGATATAGACTAGAAAATATACTCATTATAGACTAT
TTGGAAAATATTTGTAAAGAAGAAAAATGCCATATATTAACCATGCATTG
ACAATTTTTGATGAATGTATTTTCAATTTTTTTTCATGTTCTTGTTTTTT
CTTTATGTAGTTGAAACCATTTGTGATGGTTAAGAATATAGGTAACTCTT
TGAGATAGATGGTACTTAAAAATGTGTATGTTCAGTTTTGCCTCACTCCC
TGCCTTCTTACCATTGCCCGGTGGTCATATTGTGGCCTATACATTTTTTC
CTTTAAGAAATTGATTATGCTTTTCCTTATAGCTGATCACATTTTGTAAA
TATCTTACAGATGATTTGAAGTAAGTCACATTTCCTATAGAGTACCATCT
TGATAGATGTATTAATTATGGCTGTACCTATAAACTATATCTTTTATATG
CTAATATCAGTCATAGACTGATAGTTTTGTACCATGGTCATACTTATATT
TGGTTCTCTTCATATTTCTACTATTTTGGTTTTTAAAATTCTTTATAGTT
TGATACAGAACAGTGATGATTCATGATTATTATACCTTCATCCTGAAGCA
TAGCTCTTCTCAGTATAAAACAGTTCTCTATCCCCTATTAAAAACAAAAG
TTTTATTAATCTCTCAGTATGTTTTAGAGAAACTTTTTGGGAAGAATACA
TAATTGCGGTGTTTCCTGAGGCCTTCTGAGTACTATTTTTGCATATGATG
AGAGTTTGTCTAGAAATAGAATTCCTTGCCTGCTTCCTCTTCTCCTGAAA
ATTAAAGCTTTGTTCCATTCCCTGTCATCTTCAGGGTTTTCCATGTTATT
CTCATTCAGATAGCATGGGTAACCCAATAATTTTATTTCAGTCCCATTGT
CAAATGTTGCATGTGGAAATTTTTTCCAAGTTTTGGTCTTCTCTTTTGGT
CTTTCTCTTCAGTTTATAGTACTTTGCCAAATTTTGCTCTTTGGTTTAAC
TGGGTATTTTAAGGGAAAGTTGGAGGAAAGTGTTTTAATCATTAATAATT
TATCATTACTGGTGTTAACATTAATATACCAAAGATAATGTCGTTACAAA
TGTGATTTTCTAAATTGAATTTTATTTTTGAGGGCCTTTTCTGTATCTCT
TATCAACCTTCATGGGTTATTTTAGACAGTAGTTGGTTGTATTTTTGTAT
TAATGAGTTGTCGCAGCTGTATGCATATTTCTTTAAACATGTTTTTGAAC
ACTTTTATTGTTTTTCCAGCTTAACTTTCATAGAAAATTTAAGAGAAGAA
AAAGAATAAAAGCATATAATAAAAATCATCCATAATCCACTATTTATATT
CAACCACTGTTAACATATTTTGCTATTTTCCCTTTTTTTAAAGTGGAACA
CAACATGCATATGAATATATATTTAACTTTGATTCACCGGATGTTTATTG
AATACCTGTTACATGCATGAACTATTCTAGGTGCCAGACATTGTTAGAAA
TTTGCTGTATACAGTTTGTATGTTGTAATTTGCATTTACTGTTATATTCT
AAGCAAAACTCATTTTACTACAGTATTTATAACAGTTTTGTTCATGGCTT
CATAGTCTTTTTTCAGTGTTTTTTAGTCACTTATTTGGTCATTAAAACCT
GTCTTCAAAGAAGCCTAATATGTTAGAGAGATGAACAGGGAGCAGTTTAG
TTGAGAGGATATCATGACTTGAGGTATCACCTCTCTCCTCCAAAGAAAGA
TAAACTGTTGAAAACTGTTGTTCTATCTTCTGGATGTATTACAGTTTATT
AATTATTGCTCTGTTGCTGGTAGTTTATATTGTTACTTTTCTTCAGTTTT
AATTTTTATTAATATATTTGTACATAAAAATTTGTGTCTGTCTTTCGTTT
CCTTAAGATGGATTTTAGTAATGGAATTATTGGGTCAAAGAGTATAAATA
TTTATAAAACTTTGTATAGAGTCACAAATTACGTGTCATAACCTGTGTAA
CAGTCAGTATTATTTTTAAATAATATAAACATTTCTGTTATAACATCAAA
TGTATTCCATAAAAACCCTTCAAGTTTACCTATTCACTAAAAATAACAGG
ATTTATGAGAAAAATAAGGTTAAGGGCCAGCCACTCAAGATTTCTGCAAT
TAAAGCAGAGCACCAACAAAAGCATAATTGGTACCTTGTGAGATCACTTG
GGCAGCCCAGTCAAACCATTTAGCATGGCTTATGTTGCCTCAGAAGAGAG
TGAGAGATATTAACATATAGGGTGTGGTCTTGAGACAGTACAGATGAAAG
TCATTTTCTTAGCCAATGTGGTTGCCATAAAGGTGGGCACCAGAGTAAGT
GCAGCTTGCTGTGCAAAGGATGGGAATGCTCCTTTGAGCAGGGAGTCCTG
GGTACAGGTGCTCATTTTAAATATTCTTGAAATAGAGCTGAGAGGACTCC
TGCTTGGCAGCAGCTGAGCTAAATGAGGATCTTTTTCTTTCTCACTGAAA
GTTATAATTCTATTCCTGCTATTCCAGATCCCCTTGCCTAGGAAAATTAA
TATGTGAACCCACTTGTTATACTGGAATGATCTTACTTACCACTTAAAAG
CTGTGTTACAGAAACATGTCTCACAATATAACAGACTGTATTTGCTAATT
TGATGCGTGAAAATTTGAACAGAATTTTATTGGTTTCTTTCAAGCCTTTT
CAAACTGGAAATAACTACCTTAGCTAATAAAGAATGGAGTATCTGTTTCA
AAACAAGTGTTTTCAAAACCTATAGTCAGAAAACAGCAACTGTGTTTAAT
GATTATATTGCAATTTAAACACCTCAGTCTATGGTAGTGAATTTTAAATT
TACAATTTATTTTGGTGGTCATTAGGTGTTCATAGGCATTTGGATCACAG
GGCTTTAAATTGTCAGCAGCTTATAAATATTTATTTGTTGAGGGTACAAG
CATCATCTTGTAGTGAGTCATTTGTGGCAATGGCTTAATCCAGGATTATG
CAGCATTCAATTATAGCACCAGAGAGGACAGGAAGTAGAGGAGTGAGTTC
AAGATTGGGTGTTTTTTTCTGAGCTGATGGTGTATGTCTGATGTAGGCTG
TGGTGGGTGAAAGTGGAAACCTCCACTGGTAAGAAAGTAATTGAAGTAAT
TGAGGTTGAGGCCTGTAAGAAGAAACTGAGGAAATGATTGGTACAATAGA
ATGGGAGAAACAAGAGGATTATAGATACTGGAGACAGAATGACCCTGGGA
GCAGCTGTTGTTAATTGTAAAGAGCTGAAGAACTACTAGCTGTTGAGATA
CTATAGTTCAAGATTTTAGAGGCAGAGTAATTCGCATATTGCTGCTGTCC
ATGAGAAATGAGTTTCTTTGGTATGGATGAAGATTGTTGGAGTTGAGGAA
TAAAAGAGACATTGAAGGGCAGATTGAAAGTGGGTTAGTCTCAGGGACAT
TGGGAGTCTGCCCTAATGAGACATAGAAGAGGGGTGCATAGAACTTGTAT
AAACTGGTCCCAGAATCCTCAGTGAATGAGGTAACTGAGATACTGAGAAG
GATAGAGGGTGGCATAGTTTGATGGCATGATTGTAAAGAAACAGAACTTT
GGGTAAGGGTGCTCAAGCAGTCATCTGAAGTGGCCATGGCGATCCAGGGC
AGTGTCCACTGAGGGTAAGAGAATGGGCCCTTTGGGCTCCACAGGGCCAG
GGGAAAGTTGTACTTCCGAAGGATAAGGAGGTTGGTGGAAGTCAGAGGGG
TATTAGTGGGTGACAGTATAGAAGTAGATCATTGAAGGGGGATAAGGGGT
GGACGGCCAGGGTGCTAAGCTACTAGCACAAGTTGAAGTCCAACTAGGTT
TTAGAAGCCTGAGAAAGTTGGCTGACTGCAGTCTTTGCCCTGTCTTCAGT
TGAAATGTTGTGAAGTTGCTCTGTTATGTTCAAAAAGAATATTCCCTACT
TAGGAGGCATGGTTTCTGTTGAAGTTGGTAGTTTCTGTTTGGGCTGGTAA
AACTAGAGAGACCTTGAGCAATTTGGGGAGAACAGATGTTCCCAGGAGGC
TGGGAAGGAATTGGCTATATCCACCCGTCTCCTTGACAGATAAGATAAAA
TTTCTAGTAGAACAAAACCACGAATTATCAAATGACTGCATGTATGACTT
GAGGACGTGCCTGCATTTTCTTCTTTTTTTTTTTTTTTTGGTCTTGTCTG
GCTGCTTACAGGTACTTTTTATCTAGCTTAACATGGAGTTGAATTTACAG
AACTCCTAGATGCTCTTGATACTTTGAACTGTGTCTGGAAAACTTACTTA
CATATTATTTTTTGTTATTATTACCAGTAATAACAATAAATTGTTATTAG
GAACAGAGTTCTTTATTTTCCTGGGAAAGGTGTTTTGAAGTTTAAATATG
GAAGTTGGTGATCACTTAAGATTAATTTTTTCTAAAACTTTTTATGTTAG
TACAAATACTATGCTATGATTTTGCAAGCTTCTTGAGATAAGCAGAGAGG
TAAAAACCATTGAGTGTTAATTCCTTTTTTCCATCTTTTAAAAAAATTAG
TTAAACTACGTATAATGAAATACACAGATTTTAAATGTTCTCTTGGTTGA
GTTTTGAAAAATGCATACACCTATGTAACCACCACTCCAACCAAGATATA
GGACATTTCCATCACCCCAGAAATTCCCTGATGCCTCTTTGTAATAAATT
CAACAGTTTCCCTTCCACTCCCCTTTCCAGGGTCACCACTGACCTGATTT
CTATCACCATAAATTACTTTTGCCTATTCTATAACTTTATATAAGTGGAA
ATATTAGTATATACTCTCTTGTGTAGTTTCTTTCACTCATACAGTCTATG
AAATTCATCCCTATTGTTACAAAGTCTCTTATTTTCATTGCTGATTAGCA
TTCCATTTATATATATGCCACAGTTGGTTATCCGTTCATCTGTTGATGGA
CATTTGTGTTGTTTCCAGTTTTGGCTATTGTGGATAAAAGGGCTGTGAAC
ATGGCCAGGCATGGTGGCTCACGCTTGTAATCCCAGTACTTTGGGAGGCC
GAGGCGGATGGATCACTTGAGGTCAGGAGACCAGTCTGGCCAACATAGTG
AAACCGCGTTTCTACTAAAAATACAAAAATTAGCCGGGCGTGGTGGCAGG
TGCCCATAATCCCAGCTACTTAGGAGGCTGAAGCAGGAGAATCGCTTGTA
CCTGGGAGGCAGAGGTTGCAGTGAGCTGAGATCCTGCCTGGGCAACATAG
TGAGACTCTGTCTCAAAAATAAAAACGTTCGGGGGAGGGATATGAATATT
TGTGCATAAGTCTTTTTGTGGACATATGTTTTCTTTTCTTTTGGATAATT
ACTCAGAAATGGAATTCTTGGATCATAGAATAGGTGTATGTTTAACTTTA
TAAGAAACTGTGTTTTCCAAAGTAGTTGAACATTTTTTACACTCTTAGCA
ATGTGTAAGAGTTGTATAATAGTTACTCCACCTCCTTGCCAACGTTCAAT
GTTATTCTCTTGGGTGCTAATGGCTCTCACTGTGGTGTTCATTTGTAGTT
CTCTGATGACTAAGGATGTTTAGCACCTCTTCATATGCTTATTGGGTATG
TATATAATTATGAAATGTCTGCTGACATCTTTTGTCCATTTTATTTAATT
TGGTTAAAAAATTTTTGGTTCTTTATATATTCTAGAAACAAATCATTAAA
AAAAATTAACCAACATCTCTCCCTTCCCCCAGCTAAGTTCTTTCTTAGAT
AGATGTGACTGTTTTCTCCTAGTTTGTGACTTTAATTTCATTTTCTTAAT
GTTTTTTGAGGGACAGAAGTTTTTAATTTTGATGAACTTCAGTTTTTAAT
TTTGATGATTTTTAAATTTTGATAAGGTTTTAAATTTTTGATAATTTTCA
GTTTTTTCTGGTTGCTTTATGTTCTGTGTCACCTGTCCAGGAAATCTTTG
CTTACCCCAGGTTGTGAAGATTTTCTATCCTACGTTTTTTTTTTTTTTTT
TTCCAGCCCAAAGGAGAGCAAGGGAGAGAGGGAAGGAGAGACAGAAGGGG
TTGGGGGAAAGAGAAAGAGAGAGAGAGAGAGAGAGAGAAGGCAGTAGAGA
GAGTGCGAGGCGGAGGGGGAAGAAGAAAAAGAGAGAGGGCGAGGGAGCCA
GAGAGCAAGAGCGACAGAGCCCCAGAGAGGGAGCGCTCCGCTCTGGCAGA
CAGGGCCTCTTCTATCCTACGTTTTCTTTTAGAAGCTTTATAGTTTTGGC
TTTTACATTTAGGTTTATATCTCAGATTGGGTTTTGTTTGATATGTAAGA
TAGGGGTCAAGGTTTATTTATTTTTTTACTTGTAACCCTAGGAAGAGTTT
TTTAAACATTTATTTTTTAAATTTTTATTTATTTTTTATTTACACATTAT
TATTGTACATATTTATAGAGTACAGTTTGGTGTTTTGATACATATCTCTG
CTGTATAATGATCCAGTCAGGGTAGTGTATTCATCACGTGATACATTTAT
CATTTCTTTGTGATAATAACCTTGAAAAGCCTCTCTTCTAGCTATTTTGT
GATATACAGTGTTTTACTGTTAACTAGTGACTCTACTGTGTGTTAGAACA
TCAGAATTTATTCCTCTTATCTAACTGTAAGTTTGTACCCACTGACTAAC
CTTTCCCCATCCTCCACTTTCCCCTCCCCGCCTCAGTCTCTAGTAATAAT
TGCTGTGCTCTATTCCTATGATATCAACTTTTTTTTTCTTTTTTTTAAAG
ATTCCACATATGAGTGATGTCATGCGGTATTTGTCTTTCTGTGTCTGGCT
TATTTCACTTAACATGATGTCTTCCAGGTTCATTCATGTTGTAGCAAATG
ACAGAATGTCCATTTTTTTAAAAGTTGAATAATACTACATTGTGTACATT
TACCATATTTTCTTTATGCATTCATTCCTTGTTGGACAGTTAGGTTGATC
TCATATCTTGGCTATTGTAAATAGTGCTACAGTAAACATGGGAATGCAGA
TACCTCTTCGACATACTAATTTCATTTTCTTTGGATATATACCCAGTAGT
GCGATTGCTGGGTCATATGGTAGTTATTTTTTTTTCCCCCTACACACAGA
GTCTTGTTCTATCTTCCAGGCTGGAGTGCAGTGACACGATCATAGCTCAC
TGCCGCCTCGAACTCCTGGGCTCAAGCCAGCCTCCCATCTCAGCCACCCA
AGTAGTGAGGACTACAGGCATACACCACCATGCTTGGCTAATTGTAAACA
TTTTTTGTAGAGATAGTGTCTTGCTAATGTTGCCCAGACTGGTCTCATAC
TTCTGGGCCTAAGTGATCCTCTGGTCCCGGCCTCCCACACACTGGGATTA
TTGGCATGAGCCACTGTACCTGGCCTTAACTTTTAATTTTTTAAAGAACC
TCCAAAGTCAGGCCAGGCACGGTGGCTCACGCCTGTAATCCCAACAGTTT
GGGAGGCCGAGGCAGGCGGATCACGAGGTCAGGAGATCGAGACTATCCTG
GCTAACATGGTGAAACCCTGTCTCTACTAAAAATACAAAAACAAAATTAG
CTGGGCATGGTGGCAGGCACCTGTAGTCCCAGCTACTTGGGAGGCTGAGG
CGGGAGAATGGCGTGAACCCGGGAGGCAGAGCTTGCAGTGAGCTGAGATT
GCGCCACTGCACTCCAGCCTCGGCGACAGAGCAAGACTCATCTTAAAAAA
AAATAAAAAAAGAACCTCCAAAGTCTTTTTCATAATGGTGTACTAGTTTA
TAATCCTACCAGCAGTATAATAGCTGTCCTAACAGGAATGAGGTGGTATC
TCATTGTGGTTTTAATTTTCATTTTAAGGTTTTTTTTTTCTCTGTATATT
TATGCATCTAGTTGCTTTACATCGCTTGTTGAAAATACTAAAGTTGAATT
GCCTTGTTCCTCTTCCAAGGCATTTTCATTTTCAAGATCGTTGTTGGCAT
GTAAATTTCACGATTAATTTATTTTTTCAAAAAAATCTATTGAGATTTTG
ATTAAGATTACTTTGAATCTGCAGATCAATATGAGAGACTTGACATGTTA
ACAGTATTCAGGCTTATCTAAGGAGAATGGTACAGTATGTCTCTCCACTT
TTTAAGGTCTTCTCTAATTTTTCTTAGCCATGTTTTATAGTTTTCAGTGT
AGAGATCTTACATAGCTTTAGAGTTATTCCTAGGTCCCTGGTGTTTTTTA
ATGTTATAGTATTGATTTTTTAAGTTTTATTTTCTAGTTGTTGCTAATAT
AGAAATACAATTGATGTTAAAGTATTGGCTTTGTATTCTGAGACCTTGCT
AAATTCACTAGTTGGTTTTAGTAGTATTTTAGTAGATTTCACAGTGTTTT
CTATACATATGATTATGTCATCTGTGAACAATGACAGTTTTACTTCATCC
TTGTGCTTTTTATGTCTTAATACATTGGCTAGGACCTCCGGTACAAGGTT
GAATGGAAGTTGTGGAAGTGGACATCTTTGCCTTGTTTCTGATCTTAGGA
ATAAGGATTTAATAATTTACCATTGAGTATGATATTAGCTGTAGGGTTTT
TTTAATGTTCTTTACCAAACTAAGAAAGTTTCTGTCTATTCCTAGTTTGT
TGACAGTTTTTATCATTAATAGGTTTTGAATTTTGTCAAATGCTTTTTCT
GTATCGAGATGATACGGGTTTTTATTTTGTTAATATGGTAAATTATACTG
ATTTTTAAAAGGTTAACCTTTCATATATAGTTGGTCATGATGTGTTAATC
CTTTTATGTATTCTTGTATTGAATTTGCTAATATTTTGTGGAGAATGAAT
CTATGTTCATAAGGGATGTTGTTCTGTAATTTTCTTTTCTCATTATGTTT
TAGTAAGTTTTTTGTATCAATATGATGCTGCTGGCCTCATCAGATGAGTT
AGGAAGTATTCTCTTCAACTATTTTCTGATAGAGTTTATGTAAGATTGGT
GTTGTGTCTTCCCTAAATGTTTGATAGAATTCACTAATGAAGCCATTTGG
ACCTGGAGCTTTCTTTTGGGATAGGTTTTTAATTGAAATTTTTATTTCTT
TTAGTTTGGTAAATTATGTTTTTAAAGGGTGCTTCTGTTTCATCTAATTT
GTTGATTTTATTGTCAAAGTTGTTTATAATAATCTTTTTATCTTTTTAAC
ATCTATAGGATTTGTAGTAATGTTTCATTTTCATTCCTAGTATTGGTAAT
TTGTGTTTGGTGTTTTTTCTCTTTTTTCCTTGATCAGCCTTGTGAAGGGT
TTAACTGTTTTATTAATCTTTTCAAATAATACTCTTTTGGCTTCATCCTT
ATAGTTAATATCTTTATCATATGGTATTTGATCAGGGAAGGTAGCCTGTA
TGATTTTGGTTTGAATCTGTTTTTATATTTTAAAAAAATTTTAAATGCAT
GTGGTCTAATTCATGATTAATCCCCCCTCACTTTTTCATGTCCCATAATT
ATATGTTCAAATCATCTATGTCCACACTCAATTTGGTGACCTTTGATCTT
TTTTTTTTTTGATCTTTTTATTTTTTAAAGAGAGAGGGTCTTGTGTTACA
TAGACGGGAGTACAGTGGTGTGATCATAGCTCATGGTAACCTCAAATTCC
TGAGCTCAAGCCATCCTCATGCCTCATCCTCCTGAGTAGTTAGTACTACA
GGTGTGTACCACCACTCCCAGCTAATTTAAAAAAAGTTTTTTATAGAGAT
GGGTTGTTGCTGTGTTGCCCAGGCTGGTCTTGAACTCCTAGCCTCAATGG
ATCCTCTCGTTTCAGCCTCCCAAAGAGCTGGGATTACAGGTGTAACACCA
TGCCCAACTGTTTTGATCTTTTGATTTCTGAGAGGATGTTAGTTTCCAAT
TGTGATTGTGGATTTGTGTGTTTCTTTTTGCCTTTTTGTCCGTTGGTTTT
TGCTTTCTATATTTGAAGCCCTTTTTTTTTTCAGTTGTACTACTGTATCT
TCTTGTGTTTTTTATTAATATGAACTCATTAGTAAATTTTATATTAATAC
ATTTAGTATGAAATCCTTTCAACTTGAGTCCTAGTTTGTCTCATGTCAGT
ACTGCCTCATGTAATTTTCTTCTTTTTATTTATCTGACATATATTTCTAA
GTAAACAGCACCTATCTAGACTAGACTAAGACCCAGACGTAAGAAAAAAG
GCAAATTTAAATTATTTACAATTTTTCCTCTAACCCCTTTCTCCAGCACC
ACCAATCCTCCATTCCTGGATATTTGCTAACACTCCTGCCAGCAGGGAGA
GGCTTTTGCAGAAGGGTCTGCATTTCTGTCTCTGCTTTCAGTATCTTTTT
TAGGATAAATAAAAGCAGTGCTACCCAGCTAGTGCTGTGGATGGCAAGCC
ACCCAGGTGCCGGGGCAAGAGACTGAAGGCACAAGCTGTTCCAGTATAAT
AAAGAAAATATATAGAACAAGAATAGTTATACTAGAAATAGAATATAGAT
ATGATTATATATGAATATGATTAATCATTAGTTTGTAGCATTACTCTTTA
TTCCAATATTATAATAATCTCTGTTCTACAATTACAACCTAGAAAAAACC
AGGCCATACAGAGATAGGAGCTGAAGGGACATGGTGAGAAGTGACCAGAA
GACAAGAGTGTGAGCCCTCTGTCAAGCCCGGATAGGGCCACTAGAGGGCT
CCTTGGTCTAGCGGTAACGCCAGTGCCTGGGAAGGCACCCGTTACTTAGC
AGACCTTGGTCTAGTGGTAGCGCCAGTGCCTGGGAAGGCACCTGTTACTT
AGCAGACCAGGAAAGGGACTCTCCCTTTCCCCGGGGGAGTTAGAGAAGAC
TCTGCTCCACCACCTCTTGTGGAAGGCCTGACATGAGTCAGGCCCGCCCG
CAGCCATCTGGAGGCCTAAACGTCTCCCTGCGATGCTATGCTTTAGCAAT
CACGTTCCTGGTCCACTTTCATGTTCCATCCTGTACACCTGGCTCTGCCT
TCTAGGTAGCAGTAGCAGAATTAAAGTATTAAAATCTTTGATCTCTCCGA
GAAATGCATAGAAGAAATAATGACATAAGCTGCCCCCTCTCTCTCTCCAC
CTCAGCTACCAAATAGGGAAGGGCTGGCCCCCTGTCCGTCGGACACGTGA
CTTATGTGACCTTACCTATCATTGGAGATGACTCACACTCCTTACCCTGC
CCCCTTGCCTTGTATACAATAAATAACAGCACAGCCAGGCATTCAGGGCC
ACTACCGGTCTCCACATCTTGGTGGTAGTGGTCCCCCAGGCCCAGCTGCC
TTTTTTCTATTTCTTTGTCTTTTGTCTTTATTTCTACGATCTCTGGGCTC
CGCACACAAGGAGAAAAACCCACAGGCCCTGTAGGGTTGGACCCTACAAG
TTCAGCAGCTTTCCCCTATGTGGCGTGCCTTAAATTTCATTTCTGCATTA
ATGATTTTATTACACCTAGTTCAGAAACGAGAAAACTAATTTTACAGGAT
AAATACCTGTAGGAGAGTGATTTGTTTCTAGGTCTATATTCTTAGGAGGT
ATGAGATCTCCATCTTGACCTCTCTGCATACTGTGAGGTTTCAGACCCCA
TAGTGCCGGCTCAGTAAATGTGAAATTTGCATTTTGGCTGTTCTCCTCAG
TATCTTTCCCCCTCTAACTTGTGGAATATATATGTCATGTATTACTAGAG
GGACACCTGGATAGAACTTTAAATAACAGTAAATCAATTCAAGAGAAAGT
TACTCCCTGCTCTTAGTCTTTCAGTTCCTTGGATGATCATGGAGAAATCC
TCTGTGTGATTTTTTTTTTTTTTTTTACATTTCTCAAACACTTGATTATA
CCTCCCTCCTCCCTTTTTATAAACAATGAGCTAGGCTCAAGTTCACTGCC
TTCTTGGTAGGCACCAGAATTGAGCTAGAATTTAGTACATTTGTTTTTCT
TTTGTTTTTTTGATAACTCTTTTTTTAACAGTAACTGTTGATTTGCCACT
GAAAAGTAGTGATTAAATTTTGCCTTAAAAATTTTTATTTTAACAAATTA
AATAAGTTTTAAAGAAAAATATTAAATACAGGCAGGATAAAAGGGTGTGA
CAAAATTGAGATCCTGATACGTATGGAAGATTGTATGTGCCCTGGCTGAG
AAGGTGGTATGGGAGTGAGTGAAACTTCGGAGTCACTTCTCCGGCATTCC
AGGCTTTTCATTTATTTTTGTTTTGTCTATAGTACTAACCTCTTGGTTAA
CAAGCATTTATGTTTTGTGCAGAGAGCTGTGTCCTGCAGATACCATGAAA
AACATAGTGTTCTTCCCTTTGTAGTCTTGGTAGGGAAGACAGCATGTGCC
TGCAAAACACCTGGTGCAAGTGTGTACCATGGTAAATGCTATCAGAACAG
CAAGGACCTTGTGTTGTCAGAGGAGGTGGAGGTCATCTCTGGCTGGAGGC
TTGAGGCTTGGCTCAGGCTTATGTTTTATCCGAGGGGCCTTTCTCCCTTT
CATTTTGAATCTCCCTTCCTAAGTCTACGTAAATTTTTTGGGGAAGGGGC
ATTTCTTTGTAGCTGTTCCTCTAAGAAGCACTGTGTCCAGAAGAAAGAGC
ATCACACTGGCCCTCAGAAGACAGAGTGGCTCCCAGAGTTCTATCTAGTT
CTAGCACACCTGTTTTCAGCTGGATAACTGCTTAATTGTTTCAAGTCTCA
ATTTCTTCATCTTTCTTGGTGATGATAAAACCCCGTGTCTTTTTTGCTTC
ATAAGGCAGCCCATGTTAGGGCCATTTGTGATGTTCATTTGCTTATCAAT
ATTATCTTCACTCTATTTGTATTTGAAATACTGATAGGGATGATTTTGAG
AATATTTTAAGTGTTTAATTATTTTTTTCCTATTTTTTTCTAGGTCTTGA
TGCAGAACTTTATTATGTGAGAAATGACCTTATTAGTCACTACGCTCTAT
CCTTTAGTCTGTTAGTACCCAGTGAGACAAATTTCCTGCACTTCACCTGG
CATGCGAAGTCCAAGGTAAGAGAGTGGCTAACCACAGACATCATGACAGG
GTCACCCAGCCACAGAGGCAAGGGCTGTGTTTGAGGACTGGGCTGCTGTC
TGTACTGAAGGGTCCGCTGGTGATGTGATATAATAGTACTGAGTCTTTTC
AGATTTATATTTCAAATATTTGGCCTATTCCTCTAAAATTTCTTGAATCC
TTTAAAACGAAGGTGGCTATCATTTAATTGATTAGCACATGACACAACAC
TTAACATCCTTTTAGGCTCTTCAAACAGAGATCCAGACCTAGCTTGAAAA
GGGTACAGTTGGACCCAATAATGCCTGTGCTGCTGTGGTGAAAGGAGATG
GGTGTAGGGGTGGAGATTGGTAAGATAGGCTTTTTAGACATGTTGGACAG
AATGGATACAGTTCTCCCACTCATGGAGAAACGGTGTAAGAAAGGTACAG
CAGATGCAGCCCCCATGTGTCCTGTGGTGAGGCTTCTTTACCCATAAGGG
CTAAACCCAATCACTTAAGAATCTCTTGCCTGGGCTACTTGTTGCTGAAT
TCTTCACATTGCCTCTGACTGCCTGGGTCCCTCAAACATGGTAAGTTTTC
TCCTAAGTCAAAGAGTCGGAGGCTGAGCATGGAGGTGCCTCCTGTACCCA
ACCAACTTCAGTTTCTTTTATCAGCTTTATATAGGCTTTTGAATAGGATT
TTATTCAGGAAATAAAAATAGTCTTGCTACTATAAATAAGAATACCTATT
GTAGAGTTTTTAAAAAAAGTATTATAGAGTAGTGTCTGTCATAATATTTG
CTCCAAGTTATTTAAATTGGCCACTTTAATATAGATTTTTAAAAATCTTC
ATTTTTTTCTACTTAGAAAACAACAGTGATTATTGGGAAAATTTAAATAT
TACAGAAATGTTTTAAGAACTGTAAGACTAGGCCTGGCGTGGTAGCTCAC
ACCTGTAATCCCAGCACTTTGGGAGATTGAGACAGGTGGATCACAAGGTC
AGGAGATCGAGACCATCTTGGCCAACATGGTGAAACCCCATCTCTACTAA
AAATACGAAAATTAGCGAGGTGTGGTGGCACACACCTGTATTCCCAGCTA
CTCAGGAGGCTGAGGCAGGAGAATCACTTGAACCCGGGAGGCGGAGGTTG
CAGTGAGCCGAGATCGCACCACTGCACTCCATCCTGGGAGACAGAGTGAG
ACTCTGTCTCAAAAAAAAAAAAAAAAAAAAAAAAAAGAAAAAGAACTGTT
AAGACTTTGTAGTTCTATACTCAGAAGTAATTGTTTATAGTTTGGTATAA
CTTCTTCAAAGTCCCCTTTTTTCCCTTTTTAAACAAAAAAGGGATTTTTA
GACATGTATGTTGAACAGTACTTTTTCCCCTACAGAATAACTTATCTAGG
CATCTTTCTTTTTCTCTCACTCATTCTCTTTAGTGGTTTTATAATATGAG
ATTGTATGGATGTATGCTATATTGCTATCTGTGAAGTACCTCGTTGATGG
GACACATGGATTTTTAATAGCATTTTATTATTTCAAACAATGCTTCCCTT
CACCATTACACAGTTTCCCTCATTTCATTTTATAGGCAAAATCCAGGTAA
TGGGATGATGAAAACAATTGGTAGTTCATCTTTTATTGATGTTTCTACCT
TTGCTATTTCTTTTATTTAAAAATTTTTCAATCTTTGCTTTTGATGAATA
TAGAAACACAGAAAAAAATGTGTGTTAATGTGTATCCCTGTGTGTGTATA
TGTACATATATATGTGTGTGTATGTATATAAATATGTTAAGAACATCTTT
GTTCAGAAGTTTTTGTGCACTTGTGGCCTTATTTCTGAATAGTAAATTAC
TGCTGAGAAGTCAAGTGGCTAGGCCAATGTGTGTGTCTTTAAAACTTTGA
AAGATACTGCCAGATGTACCAGTGAATAAGTGCTTATTTTTGACCTTCAT
TTTCTAATACCAACATTTTAAGTATACTTGTTTGCCTTCCAGTCTCCCTC
TCCCTCCCCCTCCCCATACGTCAGTGAGTATTTTCTAAGAAAAAGGGCAT
TCTTTATACAACCAAAATACAATGATCAAAACCAGAAAATTTGACATTAT
TATGTTATTATCTAATCCACAGTAGTCACATTTTATCAATTATCACAACA
ATGTTCTTCATAGCTAATTTTTTACTAGTTGATGTCTCTTAGTTCCCTAT
CAGTCTGGAACAGTTCCGTAGCCTTTCTTTGTGTTTTTTGACCTTAACAT
TTTTGAAACTTACAGGCCAGTAATTTTGTAGAATGCCCCTCAGTTTGTGT
TGTCTGAGGTTTCTTCATGATTAGATTCAGGAATATCTCTTTGGCAGTGA
TACCACAAAGGTGCTGTCCTTCCCAGTGCACAAGGTGCCAATTTGTCCCA
TGACTGGTGGTGGTAACTCCGAACACTCTTGTCACCAGATTTCTCCCCTG
TCAAGTTTTTTACTTTATATATAGTTAGTAAGTAATTTGTGGGGAGATAA
TTTGAAACTATCTTTTAAAAAAATCTCATTCCTCATCATCCTTTCAACCA
CTACTTTTTGCATCTGTTTTTGGAACTAGTTTCGTATTTCAGCCTGTGGT
GTGGTGAGGGTAGTGTGTTTCCTCTCACTGGAAGAGGTCAGACCTACACT
GGATAACCATTCAATAAGGCTGTTATAGAAGGAATTCAAGTATTTGTGGA
GTGGTTGTTACTGAATTTTAAGTTCCCTTGCAACTCTGAGGTCCAGTGTT
TCTATGAACATCTTTCAGGCAGAGAAGCTTCTTATATTCTCCAAAAAGTT
GATAGCTAATTCTTTGTTGTTATTCTTACCTTTGATGTTGATACATATAA
AAACAAATAATAAATGTATTTCAAAATGGTGAAAGGTTTATTAGAGAATT
CTGTTCTAGGTACTTCTATTGACTTTGAACAGAGTGTGAATATTTCTGGG
CACACTTAGGAAAAAAGAATGCTGATATGAGAAGGTTTCCTGGGCAAGTG
TATGGGTAGTGATTTGTGAGTACAAAGGCAATGTGGGAAGTGGCAGTCAG
GACTGAACAAAATAAAACCTTCTTTTACTGATTCCTTTAAAACAGTTAAA
ATACACCGTTTGTTATTTGGAATGAGCTCAGGGCCCTGATGTTGCTGGTA
ATGGGTGTCTGAGTCTTAGGAGCCTGTTACAGGGGGAAGTTGGAGTGGGA
GAGAGCAGGTAGGAGGGGCTCGGGTGAATATATATGTTTGAAGGGGAAGC
AGTGTTAGCATGGTACATTTTGGGTTAGGAGAAGCCTTGGTATAGCTGTA
ACTGTACTCCCATATCATTCACATTATTTTATAGAGCATAATTTCTAAGT
TACCTCTTTGGCAGGTATAAATATTATTAGTGCACATTTGGGCTGCCACT
GCTGGTATGATATTCTTTGTGAGGCAGAAGTTTCCTGATTTTTTTTGGAG
GGTATGACTATTTTCATGTCTAGGCCAGAAATGTAGAAGGGATCCTAGTT
TCCTTCTCCTATACTTTCTAAATCTAGTTGATTCTCTCTAATAATTACAT
CTTAAATTTGTTTCCTACCTCTCTTGTGACCCAATGCCTCACACTTAGTT
TGGGCCTTCATTGTTTCTTGCTGGATTCGGAAGTGGTCTTCTAACTGGTC
TCCTTTCCTCTGGACTGACCTCTTGTTGCACCCTCCACAAAGCCACCTTA
GGCTTCTCTGTTGCTACTGGAATAAAGCTCTTGATTTCGTAGGAGGCCTT
TATGATCTGGTCTCGGCCTTTCTTTTCTGCCTCCTATCTAGCCAGTCATT
TAGTGTCATTTTTGTCTGTCTTGGAATTTCATGAATGCCCTGTGACCTGT
CACAGTGCTGCTGTTTGCCACAAATGCCGTTTTTTCATTCTTTACCTGAA
TAACTCCTGTTTACCTCATAAAGCTGTGACCTGTTGTCACCTCTTCTCCA
GCTGTGTCAGGTGCCCTTTTCTGTCTGCACAGTGCCCTTCTCTCTCATAG
CCCTAGTTATACTTTAAGAATATTGTAATAGTTGATTTTCTTTTCTTTCT
CCCTCCTACTTGCTGAGCACCTTAAAGGGTATCCTCTGTGCTTGGTATAG
CACGTGGTACTAACTGCAGAAGAGATGTGCAGGTAGAAGCCACAGGGCCC
AGCTCAACAAATGGGGAGCTTCTGAGCAGTTAGGGCTTTTCAGAGTTGGG
ATGTGCGGCTTTAGGAGTGGTGACTTTTCTATTTCTGGAAGTGTGAAAAC
ATATAGGCTGGGTACACTCGACAGTAGTTGCACAAATGGGATTTTTCAGA
TATTTTACTCATAATAGAGTGAGGTTATAAGTTTGGAAAAATGAGTGGGT
TGAATATAAACCTAAACTGATTTCACAAGTTCATCTGTAACAGGTATAAA
ATTTAGAGGAAAGTAACCGTCTTACGATGTTTTTTTAGTAAAAAACTAAG
GAAATATGTCTGTGTTAATCTAGTTTCAGGCTTCAGCCACTATTGCAGTT
CCCAATTCTAGGTAATAATCTCTGGTGATTTTAGCACAGTTGATTACGTT
TATTTATTTTAAAAATTAATCTTATGAGTAGATAATATATGCACATAGTA
AAAATTTAAAAAGCATAAAGGGTATACAGTGAAAAATAAGTCCCTTGCAC
CTTCTAGTACCATCCCTGAGCAATCCAGCCACTTAGTTCCCCTTGCCTGA
GTCAGCCACTGTTGCCTGATTATTGTCTTTCCTTTGGAAATAGTTCGTGT
GTATATAAGCTTCCATGCATATATGTATATATACATCTTTCAACAGCTAA
CATACCACCTACTTTTCTGCAGTTTGCAGAGCACTGAACAATATCCTGGT
CAAGGATCTCTGTCACTTGATACAGAGTTGTACTTTTTCCCCCTTCAAAT
GGCTGTATTACTTATTATTATATAGTTGTACTTTGGTTATTGATCAGTTC
CTGTGAGGAAATTAAGACCCAGATTTGGAAAGGGATTTGCTTAAGGTAGG
AGAAGTAGGGTTAGAGGAAGCAGAAGGTAGAGATGACCTGAAATGGGAAA
GTCACACGTGAGGGCAGGGGAGGCTTGATGAAGGGTTTGTCACCTGATCA
TTTAACCCTGAGGTGTAATCATGAACTTTCAGAAGGAGGGCCGTGGCTCT
CAAGCTCTAATACTTTATCAGAAACCCTCGGTTCTTGCTTTTTAAGGTTT
GCCAGAAGATGAAATATTTGCTTCCAGAGATCTGCAGGCTCCCTGATATG
TGTCGGTTCAACATCGTAGATACTTTAACATTAGTTTCACATATCACAGC
TTCTGGTAATACTGTTTCTCAGTGTTTTCGCCATTTTGTCTTGCCACACA
GATTGTGGTCTCTTCAGAGGCTTGAAGCTGTCTTACTCTTAGCGCATAGG
TCACACATGCCTTAGTGAGTGGTCAGGAAAGACCATTGTGTTAATTGCAT
TTTGAGTTGGGAGTAGAACTCTGGTATTGCTTCTTTTTCACAAGACCACA
CACTTAAAAGCAGGCTACTGAGAGGCTTTAACAGATAGTTTCTTCTGTAT
TCCAGAACATTCCCCAATATCCAGGAGAAACATGTAACTGTGTCTGTTAA
GTGTCAGTAGCTTTTTTTTTTTTTTTTTTTTTTTTTTTTTGAGACAGAGT
CTCACTCTGTCGCCCAGGCTGGAGTGCAGTGGCGCTTTCTCTGCTCACTG
CAACCTCCACCTCCTGGGTTCACACCATTCTCCTGCCTCAGCCTCCTGAA
TAGCTGGGTCAACAGGCGCCCGCTACCATGCCCAGCTAAGTTTTTTGTAT
TTTTAGTAGAGACAGGGTTTCACTGTGTTAGCCAGGATGGTCTCAATTTC
CTCACATCGTGATCCACCCGCCTCAGCCTCCCAAAGTGCTGGCATTACAG
GCATGAGCCACCGCGCCTGGCCGTCAGTAGCTATTTTAGATATCTGAAAA
GCGAAAATTTGCCAGAATGTAGCAGAATATTCCCACTACCCACCCTTACC
CCATGGCTGTTCTGTGCTTTCAGGAATTCGTCATCATTTTCTAGGAGTTG
CAGGGAACTTGTCCAGAGTTCCAGCAGGCACTGCTGGAGGCCAGCAGCTG
TGTCCTTTGCAGAGTGAGGTTTCTATGAGAAAGGCATTACTTCTAGTAAG
TTCAGAGAAATTAAAAGGCTATTTGGCTGTAGACAAGTGTATTCACTTTA
AATCTGTACTCTCTGTGTTCCAGAAGGAAATGGCTTTACTTACCAGCTTC
CTATTTGTAATTGCCATTTGTTTCCAAGTAAGGGAACATAAGCAAAAATG
TTTATTTTTCAGTTTATTCAAATGTTGATATTTTTGGTAATTTGTTTCTT
TTTTTTTCCTTTCCTTTTATTTTCTTTTTCTTTTTCTTTTTTTTTTTGAG
ACTGAGTCTCACTCTGTCACCCACACTGGCGTGCAGTGGTGCGATCTTGG
CTCACTACATCCTCCGCCCCCCAGGTTCAAGTGATTCTCCTGCCTTAGCC
TCCGAGTAGCCGAGTAGCATGTGCCCACCACCATGCCCACCTAATTTTTG
TATTTTTAGTAGAGACGGGGTTTTACCGTGTTGGCCAGGCTGGTCTTGAA
CTCCTGGCCTCAAGTGATCCACCTGCATTGACCTCCCAGAATGCTGGGAT
TACAGGTGTGAGCCACCACGCCTAGCCTGTAGTTTCTTTTCTGAAGTATT
TTGGCTCACTCTACATTCTGATAAACAACTGCTGTTCAAATTTCTTTGTC
CCAGTAGACTGTACATTTAACTTTTGAGCAGATCTGTTTCTTTAAAGGAT
GATTGGCAAGTGAGAGTCCTTACAGATCGATACATGCCAAACTAGAGAAA
ATTGGACTTCAATAATTGGCAATTTCTTCTCTAATCCCAGAAACTGCAGT
TTTAAAATTTATATCTTAAAGACTGCAGAGGGAAAAGAGAACTACCTTGT
GAATGCCTCCCATTTTTAGACAGTTCCTCAAAAAAGGAGCCAGCAAGGAG
GCTTCCACAGTTTCATGCTTCATATTCCTCTAAGCTGCTGATAGGACCAA
AGGGGCTGCCTTCTCAAATCTCCTCCTCAGCCCTTTGCACCTCTCCCTGT
GCCCTCATTGGCCAGCTACACTCACAGCCTCATTTCCTTCCCTTCTCTCC
TCCTTTTCACCTTGCCACACTGAGATGCTCAGGCGCCAGTCTTAGCTGCT
TTTTTAACTGCAGTTATGGCCCTGGACCCCTTTGCCAGGCCCTCTCTGCT
TACTTTGGGGTTCTGGTCTCTGGTCTGCTCCTTCTCTCCTTGTGATAACT
GAGACCTAGAATAAAGGAGACAGCAAGTTTCTGGGGAAATAGTAAGTGTG
GCTTTGAATTTGTTAAGTTGGAAGGCTGTAGGATAATTAGATAGAATTGC
CTAGTAGGTAATTGGACACTTGAGAGGGATTGAAGGTAAAAGAGAGTGAG
AGAAATTGACGATTAAGATATCCCATCAGTGGTGAAATGGGCTAGAACCC
TAGTGTACTAGGATTGAAGGATTTACATTTGGAGAGGAAAATGGACCTTT
CCTTGGGACTAGAATGAAAAGAATAGATGCTCTAATAGATCTGCCGGGGT
GTTGAAAAGAAAGGCAGAAATGGGGTTCCTCTGTACTAGTGGAAAGGGAC
AAAAACATTGAGTGTGAGGAGGTGGGGAAGGCCTGTAAGGTTGGAATAAG
ACGAGGGAAAAGAGGGTCAGTGAGGAAGGGATTGCTGAGTAGAACTAAGG
GCCTCCCCTGAGACTGGAGGTCATTGTTGTGTAAGAGCTCCTTTTCTGTA
GGATGGATTTCCTTCAGCAGTGCTGAGCAGCAGGAGTAGAGTAAGAAGGG
GTGGTCAGGATGCTAGAGCTGCCCTTTGAGGGTTTGGAAGGAGTCAACCA
GCTGAAGGTGCCAATATGACTGTCAGTAATGTTGCCTTCTTTAGGGCTTG
GGCTGTGGAGAACATGAAACTAGGTGTTCACGGCTATACTGGAGAGAAGG
GAAGTGGCTTTAACTGGCTGCTGCATCCTGCTATGAACACAGCACCTGGC
TCATAAATGTGCCTCATAAATGTTGGCTGACTTGGAGTGATGGAATCAGA
GAACTAGTTTTGTAGGAATCAGAGGTGGTGATGGGATCTTTGATAGAATG
GCATTTTTGCATTTGTTTTGGTAGTGAAGTAATTCTTTAGCGTGTCAGGA
TATTGAATACTGAATATTGAAGCCAGGGAGAACATGGCAGGAGACCAAGG
CCTCTGCAAGTCCTGTGTGGCTTATTGGTGTTGATGGCATGAGAAGTAGC
CATTATGAGTAGAAGCTGTGAGCTTCGGCCGGGCGCGGTGGCTCACACCT
GTAATCTCAGCAATGTGGGAGGCCAAGACAGGTGGATCACTTGGGGCCAG
GAGTTCGAGACCATCCTGGCCAACATGGTGAAATCTCGTCTCTACTAAAA
ATACAAAAATTAGCTGGGCATAGTGGCGTGTGCCTGTAGTCCCACCAGCT
ACTCGGGAGGCTGAGGCAGGAGAATCGCTTGAACCCAGGAGGTGGAGGTT
GCAGCGAGCCGATATCACGCCACTGCACTCCAGCCTGGGTGACAGAGTGA
GACTCGGTCTCAAAAAAAGAAGTTATAAACTTCAAAGTAGAGGGGATTTG
CATGAGGGCATGAGGAAGGGATGTCTGGGAAGGACCTAGTGATCCTAAAG
AGTGCCGACCCTACTGTCTAGGCCCTGAGGGTCAGGTGTTGTAGGAACTG
AATGATAGGAAGGGGAACAGATGACTTCTGAGGAAAGCCAGGTTGTGGGA
GAAGGTGGGAAGACATTTGTGAAGAGGCAGAGAGTGTAGGAGGCTGTTCT
GAATAGGGAGGTTCTCAGAATGGGGAAAAAACAGAAGGCCTGCAACAGTG
TTCTTGACAGACCCAGGAGGGTTTGTGTCAGAATCCCTGGGGCCAGCAGT
GGGCTGATGATTATGGAGAAGTGCAGCATGGCAATAAGGTAGCTGTTCTG
GAATGACTGCTGATGCTGCTGATGTCAGAGGAGACTTGTGCCAGGCTAAC
AGTATGTATTGCATTAATGATCTTATTTGATCCTCATAACATGTATTACC
CTTATTTTACAAAATATGGAAACTCAGAGAAGTTACAAATTTACCTAAGA
GTCACATGGTCTCATGGTTCCAAACCCAGTTCTTGCTGGCGGTGCTGATT
TCTTTTGTGCACCTACCCTTCATGTATGGTCCTGTTCAGAGATAGCATCG
GTGTAGGGATGTATAATGCATTCTTTGATGTATTGCTGGAGAGCTTGCTC
ATTCCCTGCCCAAACTTGCTTGGCTGTGGCTCTCTAGCACCCCCTGGTGG
CAGCAATGGTATTGTGATATGCTAAGGTGAATGTGAACACTTCCTAGCCA
AGGTACCTCGAAGTCACTGATGTGCTGCAGCAAGAACATTTCCTTTGTTG
GGAATGTGGCACTTATCAAACACAGCCTTTAACAGTTGTTTACGTACCTA
TCTTAACTGCAAAGGAAGAGTTTAATGTTTGCATACCCCTACAGTGTTTA
GCTAAGACACTTTAAATAGAGTGTGAAAGGAGAAACCCCAAGGGGAATCA
AATTCTGGACTGAGCCCTTCTTATTCATGGGTAACAACGCCTTCCACCTT
AGCCCAAGCGTAAGGGCCATCCCTTCCCTTATATCTGGGATGATACTCTT
TCCCTCTTTTCTTTCATCTTTATTATGTACCATTTCGTTAGCTTTTTTCA
CTAGCATATATACATATTTGAATATCTCATCTAAAAACAAAGAAAAGCTT
TGGAACACCCCCCACTTCACCATCTGTCTCCCATTCTTCTAGTTAAGTTT
CTTGAAGAATGGTTTATCGAACATCTTTCTTCTCACTCTTTTCCCAACCT
GCTGCCTCGTGGCTCTGCAAATCTCAGTCTGCCACCAATGATACCTTTTG
TGACCACTGAATGAGAGAGGGATACCATAGTTATCAAGAACCTGTGGGCA
GGGCAGAGAGTGATGTGAACTGTAGGGCTTCATTGAAAGTCTTGATGCTG
GCAGCCATTCTGAATTTCAGCTATTGCTTTGCAGTTTGAGCTGCTTCTTT
CATGAACTCTCCATAGCAACAGAGCCTTAATTATTGATGCTGATTGGTTC
TGTTAAGCCACATTGGGTAGGCCAAGTATTGATAGATAACATTGGTGTTT
TAGAGACATCTCATGGGTTGCCTGTCTTCCCTTCTGGATTCTGAATCACT
TAAGAAGTAGATCCAGAGGTCAAATGGGGATCTAAACATGAACTTTCTTT
TTGGTGAGAGCAAGACTAGAGGATGTGACTTAGATATTCTATGAGAAATC
TACCTTGGAATCTGCTTATTGTGTGAGTGGGTCTGCAGAGCAGACCTTCC
TCCACCAGCCACAAGGAGGCCCTCCGAGCTTGGCTTTCATTTATCAACCA
GAGAAGGCATTGGTAGCTATTTATGGTCTGTCAGTTACATATCTCACCGC
TTTGGTTCTTCATATCTGGCTCTCCATGGCCTCTCTGTCAGTGCTGCATA
GTTCTCAGCAGGATTTCTGACCCTAGATGGCAGGTTTTATGGAAGAGTGG
TGGTCCTGTTGTGGACTTAAAATAGAGGGTGGCTTACTTGGTTGAATTTT
TATCCAATTGAAAATTCTTAGGACTGTTTCTGACAATTTGCATTTTTTTA
TAGCTAGATTATTTGTACATATGAAAGGCTCATCCATTGATTAATGAGCC
AATTAATGAAGTCAAGCTGATAGTATCCTTCTTGTTATCACAGGTCCATT
TTGTTTATTTTTGTCTATTTTACAGGTTGAATATAAGCTGGGATTCCAAG
TGGACAATGTTTTGGCAATGGATATGCCCCAGGTCAACATTTCTGTTCAG
GGGGAAGTTCCACGCACTTTATCAGGTAAGTAAGAGTCTTCAAAGACAGA
GTTAACATACTTTTTCAACTGCATCCCCTATGGCCCCAAAACAGAAGTTT
ACTGATGTGTAGTGTGTATTACTTGATAGGCATAAGATGTATGAGTACTG
TGAAAAGTAATTTTTGTAAATATTTGCACTGTGTTCTTAAACCATTCTCT
CTTTTCTGTCCTTCTGGAACTCTCTGTGCTCCGCCCTCCCTGTCTCTTTT
TCTGCATTGCTGGTACTTACCTCCACATGAGTCCCTGTGCCTGATCTTGT
TTCCCTTTGATTAGGCACCACATTTCACCCTGTGGCCCAGATGATCTTTG
CACGGTCATGTTTGACCCTGTGACGTGAAGCCCATCAGTGCTCTCCACTC
CCATCAACTGAACATTCCAGACTCACCACCTTGTGTCCTGGATCCTTTAC
CATCTGCCCCTGCCTGCCTGTCTAGCCTTGTTGCTGCACTTGCCCCAGAG
TTGTAGCAGTTCCAGACTACATGCAGTTCTGATCTTCAAGCCTTTGCATG
TGCTGGTCTCAGTATTTGGGATCATCTTTTCCTCTTTCTGCTCGTGAACT
CCTTTCTATTCATCCTACTCATTCTGCAAGACTCAAGATCTGCACTGTCT
TTTTTCTGCCACCTTCTCTTCTTTCATTTGTTAACTTCTATTCTTCCTAC
CCCTTAGAACCTTATATTTAACTGTTTTTGCACTGATTACATATCTAGCA
ATTCCCTTGATGCATAGCATAGAGTATGGTGCAGGATAGGCACTCAGTAC
AGTCTGAGTAAATGAATAAGTGTATTATTTACACCAATTTAACTGCTTTT
GAGCTAACTTCTTAGGCTCTTGAAGTTGTGGTATTTGTAAGAAATGGATA
TTTTTAAAAATTGAAAGTATCAGCCACAATAGTGTTGTGAAACTTTTTCT
GGCAGTCATATTCACTGTTATCAGTGACATACATTAAGCTTAAATAAAGC
ACAGGAGGGCTGCTTAAATTTTTATGGGCAGTAAAGGAATTTAGAAATGC
CCATATCTTTAATGCAGTCATACAGATGGTAGAATGATAGGTTTTAAATT
TGTAGGAAAAGAATCTTAAAGATTGGGGAAAAAAATTATATGCACTGAAG
TCTGTTATCCCATTGTTACAATTATGGATTAAAATCAATTTCTGTTGAGA
AAGGTGATAGCTGACAATTTATCAGATTCATTTACAGGTTTATATTTAAT
CTTTTTATTAAGAGCAGACATACTTGCATTTTGGTAGTGAGTTGTCTTTC
CATTTGATCATGTAATTTCTGTTGTTACTATTGCTGATGTGTCTGTTAAG
GTTTGCCTTATGATCCTTCAGTTTTAGTGTACTGGAATAGCAGGACTTAT
GAGACAAAGTGGTGTATTAAAAACAATGAGCAGAAGTTGAAAACAATTAT
CTCTTCATTGATGTCTTGGTTTGTTCTTTAATACTAACTTTTATATCCAT
AATGAATTAAATGCTTAATGTCTAGAGGTGTGGTAGTCTGAAGATCTCTA
AAAACTTTTGTTTAGTTACTTTATTTAAATGTATTACTTTTAGTTTTCTT
GCAGCAAAAATAAGTAATTCTAAAATGTTTAAAAGAATCATTTTGGGGCA
TTAATGTTGATTTTTAGAAAATGTAAAATATTTTTCTTATCTTTTTTAAA
CAGTGTTTCGGGTAGAGCTTTCCTGTACTGGCAAAGTAGATTCTGAAGTT
ATGATACTAATGCAGCTCAACTTGACAGTAAATTCTTCAAAAAATTTTAC
CGTCTTAAATTTTAAACGAAGGAAAATGTGCTACAAAAGTAAGGAATTTA
TTTACCAAAATATGTTTTACATGTATGTGAGAAGGTGAAAAATAGAGTTT
GTTGTTTTTTCACAGATTTAAGTATGTCCAACTCATGATTATAGTTGCAA
GTGAGAAAAGAGCACTGGCAGGTGAAGTTTTGAGATAATCCCAGATGGTT
GGTAGCCAAGAATCTCACTCAGGCCTCATTCAGTCCATGCTTTCTCAAAT
CTTTTGTCAGAATTGCTAAGATGTTTTCCATTAGGAATAGTGCTAAAATG
GTTGAAGAGGTAAAGAAATAAGAAAAATCTGTTGGGACTTCTGAGTAACT
ATGTTAACTGTGTTTCCCTTATCAAGTTACTTTTATGCAGAAGCATTGCT
CTCGGGTGAGATAGACCTGGGTTGGAATGGCAGCTCTGCTCTTTTCAGGA
TATTGATACAAGTTACTTAACCTCTCTGAGGCTTAGTTTTTTCATCTGTA
AAATGGGCATAATACCTGTTTCAGAGGTTTGTTGTTTTTTACGGTTCATT
AAATCTAGCCTAAAACTTAGAATACCGTATGTGGGTACTTAGCAAAGTTA
GTTCTTTTTGGTTTTGAATTATCTGTTACTGAAGAAATCGAGTGGATATG
AAAGAGTTCCAGCATGCTAGGTCATAAATCAAAGAATTTTAGAGTTCTCC
TTACAAGTTTAGTCCAACCCACTCACTGTAAGTTCCACAAAGCTACAGTG
ACCCAAATTTACAAGGTCACGTAGGTAGAATTGGTTAGTATCTAAGATGG
GGGGGGGAAATATACTTTAAAAAAACCTCGCAAACCCTCAGTGCTTACTG
TGTTCTAAGTGCTTGATAGGTATTACACCTGTTTAATCCCTACAGCAACC
CTAGGCAATGGGTTCTCTCATTGTCTTCATTTTCAGAAAAGGAAGCTGAG
CTTCAGAGAATCTAGCTTGTCAAGATCAGATAGCTAAGTGGTGCAGCTAG
GATGCAGACCTGTTCTCTCAGCCTCTTTGTTAAACTTTCTTTTAATAAAT
TTCCCAATTGAAATCAGTGCTGAGTGTTATGAAAACACCTAAGGCCAACA
AGTGAGTTGAATTGGCTAATGTCTGGGTACATGCCAATAAAAAAGCTGCA
CTACATTTGAATAGACATTTTAAGAAAGAAGAGTTAGCATCTTTCAGCTT
TAGTGTTTTGGTACAATTTTCAGTTCTACTATATTCAGTTATATACCAAC
TTCCATGTAATGTTAAAGACAAAGGAGATTTCAGAGACTCTCTACTCCAA
TGTAGTCATTATATATTTGATATAAGGAGATTGGACCCTTGGCCACCTAG
GCTACCACAGCTAGCCAGTGAGGAGTGTAAAACATTCAGTTCCTTATCCT
CAGAGAGATGGGCATGACTGTACCATGGTGCTTCATCTGTTACTCTTAAG
TAAGGCAGAATGATAACCAGACATGGTGTGGCCTCTGGATCCCACCAGTG
CTAATATCTGCTAAGGTTAATGATCAGCTGATACTCAAAATGGTCACATA
CTTTCTCCATTTTCTTTAATATGTGTGTGAAATTAACATTAAAATTCTGT
GTTGGAGTAATTTTCTATTCTGTTTTAGACTTAAGTTTTTCTCTGGCCCT
CTGTATTTGTTGGTCATGGTTTTGGCCACCCTAGTCATCCAGTTCAGGCT
CATGACTCTTACTTTATTATGTGATCCTAATGTATTTATTTATCTGGTTT
TCCAGCCTACTATATTTCAAAAACTGTCTGCAACAGAAAACATCTGTACT
ATATGGAATGTTAGTAGGTGTTACTAGCAAGAAAAGTTTCTTTTATCATA
TGAGGATGGGAATTATGGGTTAGACAAGTTTTGTGACCCCATAACTCCTT
TCAGTCTTTATATTCTAATGTTCACTGTGAAGCTCCAAAGAGACACTATA
GTATGTGGCTTTTCCCAAACTTCTTGATCATAGGAACATTTTCACTGAGC
ATCTAGAAGTAACACCCTTTGGCTAACAGTGGTATAGACTATTGATTGGT
GACTGTAGTGGTGATTATCAGAATAGTAACATAGCTTTCATTATCTCGCA
ACACATACCTGTTGCTGAGTTCCAGCAATGTTCTGATAATTATAGTAATT
AAGGATTTTCATGGAAAAGAGAATTTCTAAAGCATCATTTTTCTCCTTTA
AATTCAGAACTTGAAGAAGTAAAAACTTCAGCCTTGGACAAAAACACTAG
CAGAACTATTTGTAAGTGTTACTGTATTATCTTTATCCATTATTAGAAAT
GAAAAATGGTTGACTGCATATAAACATGATTGCATGAAGCTTGGAGGCCT
CATGGTATCAAATGATAATGACACCATGCTGTTATTTTGCTGGTGCTTTG
TAGACTGGTGGATAGGTCATGAAACTGTTAGAATATTGAATATTTTGAGA
ACTAAAATTTATACGTATTTTGTAACGAGTAAGCTAAAAAGTTTTACAAG
CAAGAATATCATATATTTTTGAGTAGTCATAATTAAAGTATGACCATGAA
ATATGTGTAAGATGTTTATTAGCAGAGGGTTAGAAAATCTGGCTTGCAGT
CTGGACCTAATCTTAAGCTAATTTCATCCAGGTGAAGGTGTAAGCTTAAA
GATATTATGTTACTTAAACCTACAGAGCAGGTATGCAAAGTAGATTTTTA
TTTTTTGGTATCGGTTGTGTTTAGATATTATTACAAACTTCTTTTTTTTT
TCTGACATGAAGACATCGTTGGGTCTTACTCTTCTTGCGGTAGTGTTAAT
AGCAGTATCTTAAAAGGATACTAGATTCTCTTCATGCAAGTAGACTTTAA
AAAATTGGTTTTGAAAACTTCAAGTATATAAAAGTTCAGAGTACGCTTTG
ATGAACTCCCATGTGCCCATCCCCCAGCCTCAGTAATGATCAACACTTTG
CCATTGTTCTTCCATTGATTCCCTCCTTTTGTGGTTGGTTAGGGGTGGAG
GAGAGTATTTTCAGGCACATCCCAGGCATCAGATTATTTCACCCATAAAT
ATTTCAGTGTAGATCTCCTTTAAAAGGGCCTTTAAAAAACCCCCACAATC
ACCTAATACCTATTCATTTTGATTTATCACATATAACAAAATTAACAGCA
GTTGCTTAGTATCTCCAATAGATAGTCCATGTTCAAATTAACCCGATTGT
CTTTAAAATGTCTTTTTTTCCTCCTGAATCAAGATCCAAATGAAGCCTAC
ACAACTGTTGGGTGATATATGTTTTGTATTAATCAGAAACCACCAACCCT
TTTATTAATATTATTTATTTATTGAAGAAATTATGTCATTTGTCCCATAG
AATTTCCCACATTCTGCATCTTCCTGAAATCATTGAACATGTTTGTTTGT
TTGTTTTTCTGTAAACTGGCAGATAGATTTTGCACTTGATTAGATTTAGA
ACCAACTCTTTTGGTAAGAATAGTCCATAGATGGTAGGTACTCTGTGCTG
CCTATTTTGTCTTTTCATCACCTTGGGAGGCACAGAGTATCTGGATGTCC
CAAAGGGGAATTTTTTTGCCTGTAAAATAATGTGACATATCGTCTACTTG
CTGGAATATTTCTGAAAAGAAAGAAAAAACATTAAAAAAAATGTCACTGT
TGCCAAAGTTTACTTTTAAAATTCTGGTGACTTTGCCCATTTCTTTTATG
CTAGTAGGATATTACTTCTCACAAAGAGCAGTACTGCATATTCTTGGCCT
CTGTCTGTGTCTTCCTTGCTGCCTCTCATCTTCCCGCCCTTTCTATTTAT
TCATCCACCCAGTACCTATGGAGAGTCTGTGGTGTGTTAGGCGGTATGAC
TGAAGCAGAGGACTAAACTGTGAGCAAAGAGATCCTTTCCTCGGGGGTTT
ATAATGTAGTGGGAGAAATAGACGAGGCAATATAGGCAATGATACCAACA
TAGCATGGCAAGCTATGATAAGGCAAGCACAGGGGATTTGGGAACCCAAG
GATAGCTTCCTGAGAACAGTGACAGCTCTTCTGAGAGCTGAAGGGTGACC
AGATGGTGGCAAATGAAGGATGAATGAGGGAATGGGAAAAATGCATAGGA
AAGCCTGGAATTTGGAGGTAATTGAGCAGGGCGCATCTAGGGGGATCTGC
CCAGCAGAGCCGTATCCTTGGCTCTGCTCTGGAAAGTGCTTGGACAGTAC
TGCTAACATATGCCTCCAGAATTCAGAATTTTTTTCTGTCCCTCCCAAGA
TTGAATAGCTTTCCTGTGGCATAAGGTAATTCTTGATGAAAGTCATGCCT
TCATTCAGGCTACAGCCAAGTCTAGCTGCCCTAGAAGTCATTTGGTCTCT
ACGTTAATCTGTTCTGATTTATTGTTTGTTGATAGGAAGGTTAAATTTTA
GAAAAGTTTTATTTTTCTTATTTCCATGTGTTTTTGAAAGCTGTAAGTTT
TATCTTTTATCCTGACCTATCCCCAGAACTCCAAACTTGTATATCTGTCA
GTTTAACGTCTGTGCATGGAGATATGAGGTACCTAGTTAGTAGACTAGGT
ACTCCAAATTTAACTTGGGCAAAACTCTTGATTTTCACATCCCCAACCTG
CTTATTTTTATCTTTGTAAAGTTACCCTTATCCAAGTTATTGCTTGAGCC
AAAACTTTTCATTCTTCTTTTTCTTTCTACTTTGGTACAATCCATTAGTA
AGGTATGTTGACTGTGTTCCCAAATCCACTTACTTCTCATCTTCACTGTG
ACACTCTTGTCCAAGCCAGTGCTGTCTCTCTGAATTACTCTGGTAACCTT
CTAACTTGTTCCCTGCTTCTGCTCTTGCTCCCCTACACGGCATTCTTCAC
ACATCATGCAGTCATTTTTTGTTAAGTGACCGTGTGAAATTGCTTTGCTT
GAAACCTTGCAATGATCCCCCATCTCACTTAGGATAAACCCCAAATGCCT
CAAGTTTTGTTCAGTAGAGTTATGATGTGAGTACTCGTGATCAAATCTTT
CATGAATCTCCAGTCACACCAGTTCTACTTCTGTTTGCATCTTCCACTCT
GCCTGCTTATGTCATTTGTATCTTGCTGTGTGTTTTTTCTCAGTTTGCTT
CTGCGTGTGTGTGTGTGTGTGTGTGTGTGTGTGTGTGTGTGGCTGTGGCT
GTGTCATTGGGTTATTTTCCCTGTTGCTGAGGCTTTCTTTTTGTCTCTCT
TATTTATATATTTATTTATTTGTTAATTATTTTTGAGACGGAGTCTTGCT
TTGTCACCCAGGCTGGAGTGCAGTGGTGTGATTTTGGCTCACTGCAACCT
CTGCCTCCCGGGTTCAAGTGATTCTCCTGCCTCAGCCTCTCGAGTAGCTG
GGACTACAGGTGCGCACCACCATACACGGCTAATTTTTGTATTTTTTTTT
CAGTAGAGATGGGGTTTCACTATGTTGGTCAGGCTGGTCTCAAACTCCTG
ACCTCAAGGGATCACCCACCTCAGCCTCCCAAAGCGCTGGGATTACAGGT
GTGAGCCACGGCACCCAGCTTGCCTCTCTTTTTAAAATAATATAAAGCTG
AGTATCATTTTATTTAATGAATAGAGCCTTTAAAAACAGAAAAGCAAAAC
AATTTCTGTTGTACTCTCAAAAAAGAATAAATGTTTTTATAAACTTGTGG
ACCCTTCTGTCACTCAGTCTTCTGTGAGTCAGTGGTCCCTGGCAACAAAA
TGATGTCTGATTCCTCTAACCCAAACATTGTTGTTGAAGAAATATGAAGA
ATATATAGATCTGCACTGTCTAGTATGGTAACTGCTAGCCACATGTGGCT
TTTTAACTTTAATTACAGTGAAGTAAAATTAAAACATCAGTTCCTCAGTT
TTACTAACTACATTTCAGTAGCTTTAGTTGTACTTATATTTGAATGCTTA
ATAGCCACAGGTGACCAGTGGCTACTGTATTGGACAGTACTCTAGAGAAC
ATTTTCGTCACTGCAGAAAGTTCTGTTGGACAGCACTGCTGTAGTATATT
TTATGCACTGGTATGTTTTATGTATACCACTTGTGAGTGACTGTTATACT
CATTAGCCAAAAATTCTATTTAAAATCTTTTTTCTTTCTTTTTTTTAATC
TTTGTAAAGGTACCCTTATCCAAGTTATTGCTTAAGCCAAAACTTTTCAT
TCTTCTTTTTCTTTCTACTTTGGTACAATCCATTAGTAAGGTCTGTTGAC
TTTTGTATTAATCAGAAACCACCAACCCTTTTATTAATTTTATTTATTGA
AGAAATTATACCATTTGTCCCATAGACTTTCCCACATTCTGCATCTTCCT
GAAATCATTGAACATTTTTTTTTTCTGTAAACTGGCAGGTAGATTTTGCA
CTTGATTAGATTTAGAACCAACTCTTTTGGTAAGAATAGTCCATAGATGG
TCTGTCGCCCACGCTGGAGTACAGTGGAGCGATCTCGGCTCACTGCAACC
TCTGCCTCCCAGGTGCAAGCGATTCTCATGCCTCAGCCTCCTGAGTAGCT
AGGATTACAGGTGCCTGCCACCATGCCCAGCTAATTTTCGTACTTTTAGT
AGAGGCGGGGTTTCACCATATTGGCCAGGGTGGTCTCAAACTCCTGACCT
CAAGGTGATCCACCTGCCTTGGCCTCCCGAAGTGCTGGGATTACAGGCGT
GAGCCATCACACCTGGCCTAAAATATTTTTTTTAAACTACAACAAAAACA
TCAAAAAAATTATGTGAACTTCCACCAATATGCTACTGGTAACTGATCTT
ACAAAGAATGGGCACATGAAAAGAACAGAATACTTATTTATGTATATTGG
GTCACAAAGCAGTCATATTTGTTTAAAAAGCTAAATGTGCTTTTTGTTTT
TATTTCAGATGATCCTGTACATGCAGCTCCAACCACTTCTACGCGTGTGT
TTTATATTAGTGTAGGGGTTTGTTGTGCAGTAATATTTCTCGTAGCAATA
ATATTAGCTGTTTTGCACCTTCATAGTATGAAAAGGATTGAACTGGATGA
CAGGTATTGTACATATTTTGGGAAAGAAAAAAAATGAAAGCAGTTATTTG
TATATATGTGGGAGCCCATACACATCGATGCACAGTGGTGCAGGGAAAGG
AGGGAGGATGATTAAGCCCCAGCCAAAGGAAAAATAAGATGACCTAATCA
CTGTCTCTGGTTTCTCTGATTTTATTTTTATTTTTTAAAAATTAAGAAGT
CAGAAATTAGTTTTTTAGTTGTTGAGGAAGAAGTGGAGAACAAGTAGTTT
TTTCTATTCCCTTTGACATGACTCTACCTCAGACCATTGCCTTTTCAAAA
TAGCCCTGTTTCTTCCTTCTAGATCTTGCCTTCATCATTCTCCCTTTCTC
TCTTCCTCCCTCCATCCATCCAGTAAACATGTATTGAGCACCTGCTCTGA
TTTAAACCATTGGTTCTCAACCTGGTTACACATTAGAGTCAAGGAGAAGC
TTTGAAAACATACTGGTATTTGGGTCCACCTTCCAGAGATTTTGATGCAG
TTGGTTTGTGGTAGTATTTGGGGATCAGTATTTTTCATAAAGCTTCCTGG
ATGATTCTAATGTACAGCCAGTTTGAGATATACTGGGTTAGATACTATGC
TAAGTGCTGGGAACAGAAAGAAAGAATGATATACTTTATAGTTTCAAGAT
TTTAATCTTTTACATTAACCTGTAATTATAAAAGTATGTAATTTGATGAC
TGTGATATGTATAGTAAAGTGTGTGAATACAAAAGGTGCTTGTCCAGCCT
CAGTGAGTCAGAGATGACAAGGAGGACATTATAGTCAAGTTAGGGTGAGA
GGTACAGATGGAATGATAGGTTTATTTTCATAACAAACACTTCTTACTTC
CCTCCTCTGTGATTTCCAGTTGCTGCCTGAAGACATGCCCCAGCTTTGTG
ACTTCTTTGAAAGCCCTCCTTTATGTGGAGACGTATCATCCATACTCTTT
GAACCTTTTCTCCTCTTATTGCTGTATCCTTACTTGCGCTCCAGTCAAAT
GATCACTTATTCCTCACTTTCGCCCCACACTTTGCCCTTCAGGACTTTTG
TTCATGCTTTCTTCTTCCCACAAGCCTCCATGCATTTTCCACCAGTCCAA
ACTTGGTCCGTTCTTCAAATTCCAGCTCCATTGCTGTCTCTTTCATGAAG
TCTTTTCTAAGCCTCCCAAACACCCCTTTCTCTTTGTCTCTGTCTCTGTC
TCTCTCTCTCTCGTAGCAGTTATTGCACTTTTTCTTCTATAATTTTTTGT
GTAATTGTCTTACCTTTAGAATGTAAGCTTTTTCCAGTTAGGGAGCAGTG
TGTCTTACTCATTGTTGAGTCACCAGTCCTAATTAGTCTTAACATGCAGC
TGGGAAGCATATTTGTGGCGTAAGAATATGAAGGGGAAGGGAGCTGGTTG
GATTCAATACCTGCCCCTTAGTACTCTTTGTCCCCACTGTTTTATTTTCC
ATTTCTGTGTGGAGAAAGAAGAGAAAAGGGATACGAATGAGAAGGAAGTA
CGTATATGATTTGTTTCAGAATATGTTTTGTGTACATGATGGTGGTGAAG
TTAGGTTTAGATTTTACAGGGGAAGAAATGGCATTTGGAAGCGATTGAGG
GAACATTTTGATAGGACTTTGAGAGAGGATACAGGTAGAGAGGTGACTGA
AGAGCATGACATTTGGAAGTCAGTCTTGATATAGCCAGAGAGTTGCGTTT
TCCTTGAATGCTTGCATGCGTGCTAGGGCTGTGAGCCCACATAGGCCAGA
ATTTTGCAATAACTTTCAGATTCCATGGGGCATCTACCTCTGCCTGCTGA
AATAATTAGTTACCATCCATGAATTGGTGTTCCAGTTCTCTTTAGTTTTC
AGGCAAAAGTTTCCAGACATTACCTGAGTAAAACCAGCTTATATTATCAC
GTTTTCAGAGCATAGTCTAGTGGTCAAGAGTACGGACTCTAGAGCCAGAC
CATCTCTGTTTGAAGCCTGGCTATTCTGTTTACTAGTTTTATGGGCTACA
TGGATTAAGTTATATAGCCTATGTGTGTCTCAGTTCTCCTCTGTAAAGCA
GAAATACACACCTAGTAGGTATGTTGTGAGGTTTTAATGAAATACAGTAA
GCTCTTAAAACAGAGCCCTACGTATAGTAAATGGCATTTTATGAAGTTTT
TAATAAAGTTGCAATATGCTAGTATGTCCAGAATTGGTTCCTTCTGGTGG
GTTCTTGGTCTTGCTGACTTCAAGAATGAAGCCGCGGACTCTCGCGGTGA
GTGTTACAGTTCTTAAAGATGGTGTGTCTGGAGTTTGTTCCTTCAGATGT
TCAGATGTGTCCGGAGTTTCTTCCCTTCTGGTGGGTTCATGGTCTCACTG
ACTTCAGGAGTGAAGCCGCAGACCTTCGCAGTGAGTGTTATAGCTCTTAA
AGGTGGTGCATCCGGAGTGGTTCGTTCCTCCTGGTGGGTTCGTGGTCTCA
CTGGCTTTAGGAGTGAAGCTGCAGACCTTCGCAGTGTTACAGCTCATAAA
GGTAGTGCGGACCCAAAGAGTGAGCAGCAGCAAGATTTATTGTGAAGAGC
GAAAGAACAAAGCTTCCACAGCGTGGAAGGGGACCCAAGCAGGTTGCCGC
TGCTGGCTTGGGTGGCCTGCTTTTTTTCCCTTATTTGGCCCCACCCACAT
CCTGCTGATTGGTCCATTTTACAGAGAGCTGATTGGCCCATTTTACAGAG
TGGTGATTGGTCCGTTTTACAGAGTGCTGATTGGTCTGTTTTTACAGAGT
GCTGATTGGTGCGTTTACAAACCTTTAGCTAGACAGAAAAGTTCTCAAAG
TCCCCTACCTGATTAGCTAGACACAGACCACTGATTGGTGCGTTTACAAA
CCTTTAGCTAGACACAGAGTGCCGTTTGGTGCATTTACAATCCTTTAGCT
AGACAGAAAAGTTGTCCAAGTCCCCACCCGACCCAGAAGCCCAGCCAGCT
TCACCTCTCACTAGTAGTGATGGACAAAGTTCTTCTGCCCTTTCCTGTCC
TATTAACAATTATTTATTACCTGCCAGGCAAGCTAACAGTGGTGACCAGA
AAGCTCACATCCAGTTATCCAACAGCATTTAAGAGACATCTGCTGCTTCT
CTCTCTGCATAGGGGCCACTGGGATTACCATTTATTGAGTGTTTTCTGTT
TACCAGTACTGTACCCCAGGTACTTTATATGTATTATCTTAACACAGCCA
CCTAGAAATAGGCTTGAAGAGATTAAATAACTTTTTCAGGATTGCTCTTC
TAGGAGCAGGGCAGAGCAGGCGGGACTCAGTGTTGTAATAGCTATGTGTT
AATAATCCACTATTGTCATTGTATAAAACACTTGCTGTGTGCCAGGCACT
GTTCCCTGTGCTTTACATTCATGAACTCCGTTAATCCTCACACAGTCTGG
TGAGGTTAGATGGAGCCCATGGCTCCGTGGAATTCATTAGAAAGGGATGG
TAGTGTTCTTCCATTCCAGGCACTTAGGAGCCACTGCTTGTTATAAACAG
ATCTCATTAGAGGCAGAGAATTAACACTGCCTGAGATATGCCCATGCCAG
GGCAGTTGGAGCTCATGTGTGTGGCTTTCCCTCTCTCAACACTGCAAATT
GATCTTTTCAGTCTCTTTGTTACGCCTGATGAGTCTGTCCTGGATAGGCA
GAGCTTCTCTTTACATTCACCCTGAAGTCCTGCAGAGGGGTTGGTCTGCA
GCGTGTCTCTACTGTAGTCTTTCCCTGTTAGTTTGTGACTGTGGGAAAGT
TTATTACTCCTTTGTGGGATGCTTGAACTTGTCATCTGGCAGCATCTTAA
TTTTTCTCTTCCAGTTTCCATTATCCCCACCACCGCCCCCATGCCATTAC
TGGTGACAGAGTTTCCCTTTAAAATCTTTGATTTTATATTTAATGCCTAA
CTGTCTGACACATAGTAAAATACTCAGTAACTATTTGGTTGAATAACCAA
TGAATAGAGGGAGGAAAAAAACCATTTTGTTTTACCAATCATTCTTTGCT
TTTTTCACAATAGTTTGTGCCAGAAATGACCATTCTGTCTGGTTTATGTT
TTCTGTCCCAGGGAAGCGTGGTTTTCTCTGTTCATTGATTGACTCAGTGT
TAGTTGAGCTGTCACTGGGGCTCACTCTGGCCCACTTGCGGCTTATTGTG
CCAGGTACTGGCATAAGTGCTGGGGAAGGAGAGGTGAATAAACAGGCACT
TGTCCTGCCTTTGTGGAGTTTATAGTCTGGTTGGATGAGACAGATATTAA
ACAAAAAAAATCCTTACTGGAATGTAGGGGTCACAGTCTTTGTCTGTTGC
ATTCATTGCTGTATCTCTAGCACCCAGTACAGTACCTGACATGTAGTAGA
TACTTAATAAATATTTGTGGAATGAATGGATGAAGTGGAGTTACAGAGAA
AAATAGAAAAGTACAAATTGTTGTCAGTGTTTTGAAGGAAAATTATGATC
TTTCCCAAAGTTCTGACTTCATTCTAAGACAGGGTTAGTATCTCCATACA
TAATTTTACTTGCTTTTGAAAATCAAATGAGATAATCTATTTAGATTGAT
AATTTATTTAGACTGGCTATAAACTATTAAGTGCTAGCAAATATACATTT
TAATCTCATTTTCCACCTCTTGTGATATAGCTATGTAGGTGTTGACTTTA
ATGGATGTCAGGGGTAGGTCCCTAATTGCTTCTTGCTTCTATCATAACAG
TAAGTCTTGGATTCAAAAAAAAATTTCGTTCTGACCACTTAGCATAGTGC
TTTGCTTATTTTAGGGACCCACTTAATATCTGTTGAGGGAATGAAAAATT
GACATAGATGTCCCTGTAACGCCTTTGTGTGCCTACAGTCTTTTCAAAAA
TTCTGAGGTAGCACAAGAGTGCCACCTACTGTTAAGTTAATGGAGAGACT
GTGTCTGTGCACTTAGTCATCAGTGATGTTGGTTGCAGATAATACTTAAG
AAGTACTTTCACATATCTCATACAATTTGAGCCTCACAAAAATCCTGTGA
GGTAGATTATCTTAATCCCATTTATTTCCTCCCCTTTTGAGGATGGTAAT
GCTCCTCTTCCCTCCCCATTATTGAAGTCTGGACTTCTAACAGCTTTGAT
ATCTGGTTTGATTTTTGAGTGGTACTCAACTGTAAAACAGAGTCCTGTAA
TCCCTTTTTGCTCTAACGACTGGAGATGCATAATATTCACATAATTAATA
TAACTATAGTAGTATACTATAAATTAGTAACAATAATTTATAAAGATATG
TTTGAGGGCTAGGCAATAAATGCTTTACTTATTTATTTTTCATGTAATCG
TTTTAGCAATCTTATCAGAGGATAGTGTTATGCACACTCTGTCCACGGGA
ATTTGAGGTTCCAGAGGATTAAATAGTTTGTCCTAGGCCGTACAGCTAGT
AATCGATGATACTGGCAGTTCTGACTTCACGGTTTATGCTCTTCATCTCT
TTTCTATATTGACCTATACCCACTGCATTATAAACTCATATAGATCAATT
CTGTAATAACGTTGAGCCAAAGAAGCTCTTCCAGCACTCTGACCAGATGA
TAGCCTAATGAATCTTTATTATAGTCTCTTTGAGTTGCAAAGTATCTTCG
CAGAGTTGCTGGAGAAACAGGGCTAACTGCAGCATCACAAGAAGAAGTAT
CAGGTTGGTGCAGTAATTGCAGTTTTTGTCATTAAAAGCAATAGCATTAA
AAGCAATGACAAAAACTGCAATTTGCAATTACTTTTGCACCAATCTAATA
GATAACCTAAAATGATGGACATCTTCAGGGAAGGCATGCTGGGGGGGCAT
TTCATTAGGCAAACATGTATCTTGAGATCTATGGTTGTGCTGAGCAGAAT
GGTTTGCATATTATAAGTGCTCTCCAAATGTTTATTGCAGGACATTTCTA
TCTTAATTTTTGCTATGACTTTCTACAGATGTTTGGTTACAATAGCAGAT
AGTAGTATATTGTATTATTAGCCATAGAAAGGCTACATAAAGACTTACAG
TCCTCATTGCCTGGATGTTATATATTTACATTTTTCAGATTTGTGTATGG
GCTCTTGAAATTGATAAACATTCATATATAAATGTTATAGATTACATAGC
AGCCGTGTGTGTGTGTGTGTGTGTGTGTGTGTGTGTGTGTGTGTGTGTGT
TTTGTTTCAGAGACAGGGTCTCACTATGTTGCCCAGGCTGGCGTCAAACT
CCTGGACTAAAGTGATCTGCCTAACAGCCTTCCAAATAGCTGCAGCTACA
GGCTTCACACCGCACTCAGCTTTATTATGTATTTTTTTAAATCACCAGTT
TTTGAAAATACAGAATTATTATGTGGGAAGTGGTCAGTTAAAAGATTTTG
CATATTAAGGAAAGTTGGAGACTTGATTGGGCACTGCTGATCCTGTAGCT
AAGAGAGGCATTGAAAGACCTATCCTGCTATCTGGTTTCACCCTTCATGC
TGAATTTCTATGTTTCACGTCTCCACTGCTGCAAAATTATAAGAACTAAG
GCAGTGAGACCGAAATGTACCCACGAGACAAGGTTTCTCCCCACTGAAAA
GACAAAACTACATGCTTGAGCAGCATTACTTTGCAGTAATCTTGAACACA
AGGTTTACAATTATTTGAACCTATCATCTTCTTGGTGTCCTGTATTATTT
AGAGAATATGGTGTAGTGTTTCAAGTACTAGAGATTTCCCAGAAGTTGGA
TGAAAGGGAGGGATTGGGGAAGTAGAAGAAGCAAAATGTATTGTGTTTAA
ATAAACTTGAACCAAATTTGATACTTTTTTTTTTTTTGAGATGGACTGTC
ACTTTATCGCCCAGGCTGGAGTGCAGTGGCGTAATCTTGACTCACTGCAA
ACTCCACCTCCCAGATTCAAGCGATTCTCCTGCATCAGCCTCCCGAGTAA
CTGGGACTACAGGCACGCGTCACCATGCCCGGCTAATTTTGTACTTTTAG
TAGAGACAGGGTTTCACCATGTAGGCCATACTGGTGTCGAACTCCTGACC
TCCAGTCATCTGTCCGCCTTGGCCTCCCAAAGTGTTGGGATTACAGGCGT
GAGCCACCGCACCTGGCCAATACTCCTTTCTAAGCTCCGAGCATGTTGGC
ACATTTGATTTGGTTTGCTCTTTCTTTATCTGTAGGCCATTTGATATTGG
GCCATCATTGAAAGAGGCCTGTCTTTCTTCTTCTTTTCTAAAAAGCAATC
TAATTGAATGACATGGGCTCTTTTTTTGAGAGATGATCCGTAGTTCCCTA
ACATATTTGTGTGTGAAGTTTAGGACAATTAGCTGAGGTGTCTGAGCACT
AGTACCTACATTTCTGAATAATTTTGATAAGATGAACTCGCTTAATGATT
TTGGAGATAGAAATGAAAGAAAAAATAGAGCTCTATAGGAGTTAGATTAA
AATATCAGTATTGGAACATCATGAATTATTGTGATCTCCGTAAGGATTTT
AGGCTGGTTTTAATGGCTATGTGCATTTTGTATGTATGGAAAAAGGAAAT
TTCTCATTGAAACTGACTTGTCAAATATTTCTCTCAAAAATTGTTTGCAG
CATTAGTGCCAGCAGTAGTTCCCAAGGGCTGTCTCAGCCATCCACCCAGA
CGACTCAGTATCTGAGAGCAGACACGCCCAACAATGCAACTCCTATCACC
AGTAAGAGTTAATTTAATTCTAAAGCCAGTTTACTCAAGTTGTCTATGCT
GCTTCCCAGTGGTCTTGCATGGTTAGTGTACTTGTGGGACATGTTGTGTA
AATCGTATCTAAAGTATGCAAAATTAGGTATGCTCTTCAACTTTTGCTTT
CTAGAGCATTGAATGAAACATTCTAAAATTGAGGTATCAAAAATGAAATT
TAAGTTTGCTAGAATTGGTGTGGCTTTCGGGAGATTTCCATAGATTTATT
ATACTAAAAGGGCTACTAAGTCACTTCTATAGAAATTCAAAAGGAAGATT
TTTCAGGTAACTATATGGAATTAACTATTGAGCTGCATAAAGACAGAGAC
TTTAGACTGTAAGGAATTGTTTGATGGTCTTATGGGCCATGGTGAAGACT
TTACATTGTTTTTTAATCATAATAGGAAGTTAGCTACCACTGGAAAGTTG
AGAATGTCGGATCTGATTTCCTTTTTTGGACTGGCATTTATTCTTTAGTA
AAATAAGATTAAGATATCTGTCCTCTACCCAGTGCCCTATTGTGGTTTTC
CAGTACTGTCTCCCATGAAAAAGAACCAGGATTTTTTGGAGAAATGGCTG
ATTCCAGGTCTGAGTTAGGAAGTGAACAAGATAAGCCTGGGACATGTTGC
TGAGTCAGATATCAAGGAAATAGCTAAGACTTGAAGAGCTTCCACTGGCT
AAAGATAGGACCATTTGATCATCAAAATGAATAATGACTATAATGAATTA
AAACGTAAAATGTGTAAAAATCCACAAATTTGTAATAATACATTATTTAA
CAAAATTGTTCATTATGAGAGGTGACTAAGGCTCCAGTGAATACCTTACT
CTACCAACTGACTACATGAACTGTATTTCAGGTTAACCAGATGGTGCTGG
TGGATGAGAGAAAATTTTTCTTTACAGATGATTCTCAGCTAATAAATGTG
GAAGAAATTATATAATTAGAAAATCACATGTGCAACTCCTGATAAAATAG
TTGATTCAGAAAAAGATCATCAGTGATTGAAAGGAAAATGGGGAACTCTG
CCATTGAGGGATCAGGCTGTCACCACCTGAATACACTGATGAACTCAGCA
TTACTAAAAGTAGGGGACCTAGGTATTTCGTACCCAAGGATATGATGTAC
CATTCATGGAGCATTCTTTTCTGTCCCTGCTGCACCCCCTACAAGTTAAC
CTGGATCTGATCAAGGTTATAGACCTGACTTACAGTTTACAGGAAATACT
ACTTGAAGAAGTAGAGGCCAAACATTTTCCAGGGTAGCCGACCCAGTTTC
TTCAATAAGACAGTGGCAAGAAAAAAGGAGGAAAGAAGGCATTATACAGT
AAACAAAATTTAAGGGAAATAATAATAGCAGCTACTCTTCCCACTAATGA
AGAAAGAAATATATGCTTTTTAGGAAATACGATAGATGTGAAAGTAGTTT
AAGCTACTCAGAAGAGGCACATCATTGCTTTTAATGTTTAAATATCAATC
TTTGCTCATTAGAATGGATTCTTAATTGTTATACGTGACCTCAAAGAGGT
AGGGAATTGTTGTTATACATGCTAAAATACTTTCTTAAAAAAGTCAAAGG
GGCCGGGTGTGGTGGCTCTTGCCAGTAATCCCACCACTTTGGGAGGCCAA
GGCGGGCGGATCATGAGGTCAGGAGATCAAGACCATCCTGGCTAACACGG
TGAAACCCCATCTCTACTAAAAATACAAAAAATTAGCTGGGTGTGGTGGC
TCGCGCCTGTAGTCCCAGCTACTTGGGAGGCTGAGGCAGGAGAATGGCGT
GAACCCAGGAGGTGGAGCTTGCAGTGAGCTGAGATCACGCCACTGCACTC
CAGCCTAGGCAACAGAGTGAGACTCCGTCTCAAAAAAAAAAAAAAGTCAA
GGGAAATAAATTTATTTATCTTTTAAAAATTGTTGCCATCAATAAGAAAT
TTACTTGTTTGTAATCATGGCCATTACCGTAAGGAGCTAGATAATTTACT
TACTACATATATATATACTGAAAACAAACACTTTTTAAAAAAGTCAATAG
AAATACATTTGTTTACAGATAGAAAAAGAGGAAGAAAAAAATATTTGAAG
ACATAGTAGCCAAGAATTTTCTAAGATAACAACGCAACAGATCACAGATC
CAGGAAACTAAGAGAACTCCAGGTAGAATTAAACATTCACATATACTCAC
ACACCTAGACAGTCTATCACAGTCAAACTGCTGCCAATCAGTGGTTTCAG
GTGGTGTCAGTCTACATGCATTGTAAAGTTGCCCAAAACTTTTCATGTGG
TTCTCCTGGCAACCATTGATGATCATTGCATAGGTCCACTAATTTTGGGG
GGAGGTTTCTTCCCATTATCTGGAGATGCCATTCCAACTTCTACTTGCTA
AAGCCAGATTTTCCTTTTATTCTAACATTTTTTTTCTTGAAGTGAAAACT
AATTTGTGGCTGTAAATTCTGTTTTTACTGTCATTGAATTAAGAATGAAT
GGGAATTAAGGGAACTTATTAAATTTGAATCTCACAATGGTTTTACTAGG
ATGTGGTAATACCAGAATAATTACATTGATTATGTATTTTGTTTGAAAAA
AGCCTTTTCAAACAAAAATAACTTTCTGTTTGAAACTTTTTGTTTGTTCA
AACAAAACAACTTTTCAAATGAAGAGATGTTTGAAAAAAATCTTTTCAAA
CATGTCTTCATTTGAAAAGTTGGTGTGTTATGAGACTATATTAGAAAACC
TTTTGGGAGAAAAACTGTCCATAAGAAGGATTAAATGAAATTTCACAGAT
CAAAGCTTGTTTCATTTGGCTAAAAGGCAATTGAAGTGGTGGTCATGGTA
ATACTATTTTTAGGATTTCTTGTTGCAGTTCAGAATTTTAACATTTTGGA
GTAGTATTGTGGTGGGTGAGGAGACATTTCTTTAACATTTAGTGTGGCCT
TCAAATAACTCTCCTAGTTCATATATATGCCTGTTTCCTTTTATGATTCT
TACACTCATTCCTGTGACCTGAGTCCTGAGGTTCTAACTGAACTAGTTGA
CTCCAATACCATAATAAGGGTATTTATATTGCTTGGTTTGGCTTTGTTGG
CTCCTTAGGTTATCCTACCTTGCGGATAGAGAAGAACGACTTGAGAAGTG
TCACTCTTTTGGAGGCCAAAGGCAAGGTGAAGGATATAGCAATATCCAGA
GAGAGGATAACTCTAAAAGATGTACTCCAAGAAGGTATTTATTATCTCAA
AGTCGGTTTTTTAAGTATGATTTAATGTTACCACTATAGACACTTTTTTC
AAAAATTTAAAAAATTTCATCTCATTTTGTGTCTAAGATAAGGATAGGAA
GCAGTGTATGGTTTTTCTTTTATAAAAATTATTTTCATTTGCTCCTATAC
TTGAATGGGCCTTGTACAACTTTGGTGCAGTTCTTGATTTTCAAGTATAT
TAAAGCACGTATATTATTGTTGGCTATATTTAAGGTGTATTCTCTGTTTG
GTTTTTATTTAAAATGGCCACTACCTGGCAGGTCTCTCTGAGATAGTCTA
AGTTACTTTCTCTCTGACGGATAACTTGGGGGTTGTAGCGGAAGGAATGG
ACATGGGCTTGGGCTTTGGTTTTGGTCCTTAGTTCCAATCCCACATTTTA
TTCTCCGTGTGGCTTTGGAGAAATCACGTAATGTCTTCAGTCTCCAGTTC
CCTCCTCTATAAATTTGGACTGCGTTGTCATCTCTTAGACTAGTTATAAA
GATGAGATGAAATTAGGTACAGTAAATGAATGAACACATAATAGTGTTTG
CCAGGTGTTAGTATGCTTCCCTTCACCTCTCACTGGGCCTTGCTTTCCTT
TTTGAATTGGAGGGTATTGATTAGGGAGATGCTGAAAGAAGAAGACAGAA
ATTAATAATAAAATACTGCACTGAGCTCTTCATGTTGTTAAATAGAGAAT
TACTTTTTTACCTCCACTGTAATTCAGTTTGGAATGCTTTGGATACTTAT
GAGTCCTCCTTATGTATTCCTTTGCTGGTTGGGACACATTTAGGTCAATC
ACAGCTAAGTCAGGTACTTAAACTGTCCCCAGTAGCAAATGGTAATTAAT
GGAAGCAGATTTTCTGGTATGTCTTAAAATGCATGATTGCCACTTGTTAA
TAGTTGACTGTTTTCTAAGCATAACTAGTTTGCAGATCTTTTTGCAATCT
GAAGAGAGTAAGTAGACCAGGTAGCATTTCCTAAAGAATAAAAAAAAATG
AGAGATCAATTAATGTTATATTTGCAGTTTCTTTTGGTCTCTGAATGGGA
TAGAATAGGCAATCTGTTGTAGTGGTATGATTTGAGAAGAAGGAATTCTG
AACCAGGTAGAGTTTTTCTTTATTGGTCTTTTCAGGCCCATAATAGTTTA
TTTTAAAATTCTTTTGATTTCAGAAAAGAAAATATGGTGGCTGATTAACA
TTTAAAAAGGTGGTCAGGTTCACTGGAAATCTGATAAATGCAAAGAAAAC
AATAGTTACATGCGATTTTTTTTTTTTACGTTAGTTTGTGAAAAAGTAAA
AAGATAATTTCTAGGTTGACGGGTATGGAGGAACACATGCTCTTTTACAT
TGCTGGTAGGAATGTGAATTGCTGTAACATTGTGGTAAAGTAATATGTAC
ATATATATTTTTGTGTATGTTCCTTACTTCCATTGTTTTTAAGTATATGT
AAATCTGTGTTTGAGAGTAATATTTAAAAATGGGACTAGGTACTGGGAAG
AGGAGACAGGAGGTTTTGCAGTGCTTCAGTGCAAGAGAGTTAAGAAGCTC
AGTCAAGCAAGGTGAACTTGGCTGGAGAGAGAAGGGAAAACCTTTGTGAG
GAAGACAGGTCAGGGTGTGTAGCTCTATGTATTGACTAGAGTGTTTGAAG
ATATTTACTCAGCTTCAAGCAAAATGTCTTAAATGAAGATTATCCAAAGT
CTTTTCTGCCTTGAGCATACAAGTTACTCTGTCTCTAGGCCACAGTGAAA
CCTGATGATACATGGATACCAACAACTCCACCTCATAGAGTGGAAGGCAT
TAGAGCAAGCCTATTTGTATGCATGGTATCTGCAGCAAAGTGAAACTATT
TGCAAGCTTATCTTTTAAATTGTGAAGGAAGGAAAGTAAACAATTTTCTA
GTGAGAAAGAGTAAAATGAGATTCATTTTCAGTGATCATCTTGTTTGGGA
GTTTTCTATGGGCAATTACAAAATTGGTAATAAAATCTAGATGGATTTTC
TTAGTTCCTTCCCTAGCACCCGCAAAGGTCATAACTAACTTCTCTTATTA
CTATAAGAGTTTTTGTTTTTTGTTTTTTTTTTTTTTTTTTTTGGCGGAGT
CTCGTTCTGTTGCCCAGGCTGGAGTGCAGTGGCACAATCTTGGCTCACTG
CAGGCTCCACCTCCCGGGTTCATGCTGTTCTCCTGCCTCAGCCTCCCACG
CCCGGCTAATTTTTTTGTATTTTTTTTTGTATTTTTAGTAGAGACGGGGT
TTCACCGTGTTAGCCACGATGGTCTCAATCTCCTGACCTCATGACCCGCC
TGCCTCGGCCTCCCAAAGTGCTGGGATTACAGGCGTGAGCCGCGCGCAGC
CTCTTCTCACTCTAAGAGTTTTTAGGAACACATTTTACCAGTGAACCGAG
AGTCTAATTGGCTTGTCTGAGACCTTCTGAGGAGTGAGTGGTAGAAGGAA
TGGAACCCAGGACTGTCTGGATCCAAAACTTGTACTCCCTGGCCTTCCCT
TGAAGCTACTTTTTAAGGCTCTGCGGTAGAGAGGAAAGTTCATTTTTGTC
TTTTCGTCATTTCTCTGAGGCTAAGCCTAGAAACTCATAAATGCCATTGT
GGATACCTCATTACTGAGCTCCAATGCTCCTGGCTATATCATTAGTTTAT
TTAGTATAATTCAGGAAAATTTTCATATACTTAGTTTAGATATGGTGTTT
CTGAAAGCAAGTTAAGATGTGTAATAGTCACAAAAAACTTTTAACAGTCG
TTAAAACCAAAGATTTGGGTCTTTTTACTCTAGGGCTACAGTAATGTTGA
TCACATATGACCTTGTTATTTTTGTTTTATGTTATATTGTGCCAGAAAGT
TTTGTACTATAATTTTATGAATGTTGTATGATCTCATTAATTATTACTTT
TCCTTTTAGGTACTTTTGGGCGTATTTTCCATGGGATTTTAATAGATGAA
AAAGATCCAAATAAAGAAAAACAAGCATTTGTCAAAACAGTTAAAGGTAG
GATCTTTTTTCCATAGTATTTCCATGATGCTCTTCTGTCTCAGGTGGCTC
CTGCCAGCAAAATTCTGTCTCAGTTTCCCAATGTGTGCCAGACCTTTTAA
ATCAGTCAGTGTTAATTTAGCTGAAAACAAAAATTGTCTTATATGAGCCT
AATTGTTTTGGAAATTTGGGTTGTTCTCATTAAGTATAGAAAAACAGAAG
TCTGTTAAATTAAGACATAAGACATTTCTTAGATATTACTCAGTTAATTG
CAAGTCAAACTGCATCCACTTGATTTCATTGTTACTATGTTTAGTGTTGG
TATACTTTTGCAGGTATAGCCATAGTTGGAATTGCTGGCCTAAGGGTGTG
TACATTTAAAATTTTAATAGAGCCCAGGTGCAGTGGCTCATGCCTGTAAT
CCCGGCACTTTGGGAGGCCGAGGTGGCCAGATCACCTGAGGTCAGGAGTT
CGAGACCAGCCTGGCCAGCATGGTGAAACCCCTGTCTCTGCTAAAAATAC
AAAAATTAGCCAGGCGTGGTGGCTCACGCCTGTAATCCCAGCTACTCAGA
AGGCTGAGGTGTGAGAATTGCTTGAACCCGAGAGGCGGAGGATGCAGTGA
GCCGAGATCATGCCACTGCACTCCAGCCTGGGCGACAGAGACTCTGTCTC
AAAAAAAAAAAAAAATATATATATATATATATTGTTAGATTGTCCTCTAA
AAGATAGCACTGGTTTATATTGTCATGAACAGAATAACTGTTTTCACATA
TTCTTCTTTTGGGCCAGCGTTTGAAATGTGACGCCTAATTTTTAATTTAT
TGATGTGTATAAGTTATTTAATTAAAGATTCCCTATCAAGATACTACAGT
GTTATTTATGCAGGCATATGGCTTATTTCCTATTACTGGTAGATTTGCAA
GACTTTAAAAAATACTAGAATTTTAATTACTTACAACAGATGCTTGTGAA
AGCTTAAATAGTTCTGAATTAAGAGCTGGGTGCAGTGGCTCACAATTGTA
ATCCCAATGCTTTGGGAGGCTAAGGCAGGAGGATTGCTTGAGTCCATAAG
TTCAAGATCGGCCTGGGCGATATAGCAAGACCCCCCGTCTCTACAAAAAA
AAAAAAAAAAAGTAGCTGGGTGTGGTAGCATATACCTGTAGTCCCAGCTA
CTCAGGAGGATGAGGCAGGAGGATTGCTTGAGCCCAGGAATTCAAGGCTG
CAGTGACCCCTGATCATGCCACTGTACTCCAGCCTAGGTGACAGAGCAAG
ATCCTGTTTTAATTTTTAAAAATGATGATAATAATTCTGAGGTAAGTCTT
ATTTGCTTATCTAGAGAGAAAGAAAAATGTGATAATTTATAGGGTGCTTA
ATGTCTATCAGGTCTGATGTAAATTGAGAAGACATGCTAATAATATCTAA
AAACAAATTTAGCACTTTCCCTTCAGAAACTGAAGGGAAGATTAAATTCA
GTTTTATGAGTAGAAAGTGATAAAATTTTATACAGTTTATGAAGTTAATT
TATAAGAACTGTTTCTAAATTTACTTAAAGGAATACATGGAAAGTATGAA
GGGTTTATGTTTATTGGTTGTAAAAAGTTGGGTTATACCAATCTAACAAA
AAATCTTTGGAATTGCTTTTAATTGAACAAAATTGTTTGTTTAAGGCTGC
TACAAATACATCCATACACACACACCTCTTTAATATTTTCTAAACAAGTG
GTTTAATTTACTATATCATTCAATATTTATTTTATTCATTCACCCCTTCC
CAGCACCAAAAACACTTTTAAAGGGTAATATTTATGTGTTAGGTCCATCC
TTTCGGCTAAGACCAAGTGTAAGTTAGGTCCCAGTAGGTTTTCTGACTAG
GAGAGTTTACATATTACTTTTTGTAAATGTAAAATTTGAACTATAAGGTT
ATTAAACAACAGCCTTTTGTTACTCTTTTCTCATTTTTTTTTTAAGGAAA
CTCCTTAATTCAAATATTTCTTCCGGGGTTTGTAAGTATTCTATAGACTG
TGTAGAACTGGGCTATTATTGACTTGTTCTGAATTAAGGAAGGAGGTTTT
TATACAAAATTGGGTTGATTTAGGGTTGGAAGGGGAGATAAAAGGAAATA
ATAGTGATAAGAAAACACAATGAGTATACTTGGACTGTTTCACAAGATAA
GACTTCTCTTTCCGTATATGCTTGACTTGTAGTCACTGGTTGTTTCTGTG
CTTTACCACTGTAATTATAATGTTCTCTGTGAGCTGTGCGGAAATAAGAG
AAAGCACAGTGAGTCCTTATGGGGGACAGAGGGGAAGTGCATTGCCTCAA
ATTTGTTAGAGAGATAGCGCCACATGACTGTCTCTGAGATTGATTATTTT
GGAATGCTGTTTTTATGGTTTTAGTTGTAGCCACATAGAGCTGACCAAGG
TCTTTCACAAATCTGTATGCTTTGTTACATGTTGCACAATTAGAAGTTTA
GCATAAAATAAAAAAACAGTGTTTTAGAAAGCACTGACCTTTGCCTTTAG
GCATAAGCAGAAAATTGGAGTTTGGCTTTTAAAAATATTCATAATTTTAT
ACAGAGACTTCATAGATACTCCAGTATAAATTTCCAGAGATTGAAATTAG
CAACCCGAATCTTACACACTAAATATTGTGAAATTGAATTCCATAAAATA
AATGAATCAGTTTGGATTTACTGGTATTAAGTTCTTATTTTCTTTATTAT
AGCTACTAAATTTTTATAGTTTTGTAAATAAGCAAGTGTTTGTTTTTAAA
AATCATTTTGTGCATCCTTGTAACTTTTAGAATATATTTAATAGTACCCT
TTTCAAATGTGTACATCTTGAAGATAGACCTTTCATTATGTAATTAAAAG
ACTAGGATGAGACTGGGAAAGGAATCCAAGAAAACGAGTTTTTCTTACTA
AATGGATAAAGTTATTTTATCTTTAATTGAGTTTTCTTAGATCATCATTC
AAGTGTCCTTTGCTTTAATAGAACATGCTTATCAAAATAAAAACTTGAAA
AAAGTAAGTATAAAATGACACATTCTGAAATTACCATTTGTTGTACTTAG
ATCTCCTTACCAGTTGAGGGAATGGTAAAAGTCTTTTTAACTGGGCAGGG
ACCTCCAACTATTACTAGTTTTCAGTCTTGAAAAGATCCTACTAGCCTCC
AGTAGTCCTGGCCATAATTTCATTAGAAATTATTTAGGTTATAGAAAGAG
TAATAAAGGCACATTTTGCTATTTACTAAGAAATCCCACAAGCAGTTTGT
AGACACTTAGAGTTTATTCCAGCAGACATTGATATGAAGCACCTTTTCCA
AAGAAGTACCGAATGTTTCAGATCCTTGGCTTTTTGTCTACCCATTTTAC
ATTCCTACAAAAGTCTTAACTAAGAGAAGTTGCTTGGAAAGGGGACACTT
AGTAGCAAGTAATGGAAGTGAAGCAAGACGAAGTGAAGCAGGAATGAGAT
TCCAATGGGTAGATGTGGAAGTAAATGAAATTTGAGATTTCTCAGCTTTG
ACAGTTCCACATTTAAAGACCAAATGTGACAATCATAGTATCTGAATTCA
GTGGGGTGGGTTTTATTGTTGGTTCAGCATTTTCCTAGGTGGGTCTAGAA
TATGAATCTTGGCTTTGCCAGTCTTTTACATTTCTCTCTTCCTCTTATTG
CATTGGTCCGAAGCTTTATAAATTATTATTTAATTAATTAATTAATTTAT
AGATGGGGTCTCACTCTGTCACCCAGGCTGGAGTGCAGTGGCACAATCAT
AGCTCACTGCAGCCTCAACCTCCTGGGCTCAAGCAATCCCCCACCTCAGC
CACCTGAGTAACTAGGAATACAGGTGTGCAACACCACACCCTGCTAATTT
TTTTTTTTTTTTTTTTTTTTTGTAGAGATGGGGTTTCACCATGTTGCCAG
CCTGAGCTTTACAAATTATTATTGCTGTTTTTACTACTGATGTAAAAATT
CGTTATGGGTTTCTTTGAAAGGGTTTTTAAGAATATACTGTTAAGTTTGT
TTTAACATTTAAAAACTATTTTTTAAAATTCAAACCCACTTCTTATTTGT
TTATTATTATTATTTTGGGAGACAGGGTCTTGCTCTGTTGCCCAGGCTGG
AGTGCAGTGGTGCAATCATGGCTCACTGCAACCTTGATGTCCTTGGCTCC
AGCAATCCTCCAGCCTCAACCCCCTCAGTGGCTGGGACTGCTAGTGTGCA
CCACCAGGTCCAACTAATTAAAAACACTTTTTGAAATAGAGATGGGGTCT
TGTTGTATTGCTCAGGCTGATCTTGAGCTCCTGACTTTAAGTGATCCCCC
TGCTTCAGCCTCCCAAAGTGGTGGTATTACAGGTGTGAACCGCTGTACCT
GGCTGCATTTTCAATTTTATAATAGAAATATTCTTTTCAAGCAGTGGAGA
AATTCCTCTATACTGTTAAGGTCGTGGTCCCTATAAGATGAGGAATCCTT
GTGGATTTGTGCAAATATTTGAAGGCTTAAAAATTCCTTTGGAGATGTAG
ACTTAAAGATATTGGTAAATTGCATTATCATGTTTTTGTTTCAAAAACAT
TGACCTAAAAGTTCATTTGTTCTGTTTTGCTACTTTGGACCAAAAACCAT
GAGTTTAGAAACTAAAACTAAAATGGCATAATTCTAATTTTGGTTATCAT
ATGAAAGTTAACTATATTTTTACTACTGTATTATTAGTTCTTTTTGTTTG
TTTTTAACCACAGAGAAGTAAAAAGAAGAAAGCAACAGAATAGGCCATAA
TCTCCAATCATAACTTTTGTAGTCATTTTATACCTGTAATGTTTGTTAGG
TTAGGAAGTATAAACAGAACAGAAAGGTACAAAACCTAAATTCTCCCATC
TTGAAAAGTAGCCATCATATGTAAACAATTCCTTAATATGTAAGAAATAT
TAGGATATGTAAACACAAGTTGTACCTTGTTTTTGAAATTTAATATACCT
TAGAGATCGTCCTCTGTCAGTACATATCTCATTTTTCAAAAGTAGCCACA
TAGTATCCCATTGTGTCAATTTAATTAGTTCTTTGTTAGTGGGTACTTTG
GTGGTTGAGTTTTTTTGCCATTGAACAGTGCTCTTATTACATTGTACATA
GATGAGCTTTTGTGACTATATCTGTAACAGAGACTCTTAGCAGTGGGATT
GATGATGGAAGGTATATCTGCATTTCAAAATTTTGTTACATGTTGCTTTC
TCCATCTATCCAAGCTACTATTGATGAACCTGTTTTCTCACACTCTTGCC
CAAATTCTTAATAGTAGGTAGTGGAATTTTTTGTGTCTCAATTTGACAGG
GGAAATGGCATCTCCTGGTTTAATTTGCATTTCTTAGGACTGAGGCTGTG
AGTCGTTTTGTAATCTTGTGTGTCATTTGTTTTTTATTTTCTCAGTGAAC
TGTGTGTTTATATCCTCTGCCTCTTTTTCTTATTGATTTATATGAGCTAT
TTAAATAGGAAAAAAGGAAGTTAGCAGTATATATGCCTTATGTGTTGCAT
ATGTTTTTCCAGTCTGTTATTGCATTTTCTGCTTTGTTTATGGAAACTTA
ATTGATTTTAATATAAAAATTTCTGGGAAAATTGACCTATTTAATGAGAC
TGCAGTAGAATTTTTATCTAGATCTATTTAGATTTACCTGTAACTTATAA
TGAAAACCTATGTACCTTATAGTTCAAGATAATTATTTACTAATAGCAGA
AAATGTTAATGCCATATATGTAGTATTATTATTCAAGACATTTTCTTTTC
TTTATATATAGATCAAGCTTCTGAAATTCAGGTGACAATGATGCTCACTG
AAAGTTGTAAGCTGCGAGGTCTTCATCACAGGTGAGAGGAGAAACCACCA
GCATTTCAGTGTTGAGCATGGCAACACTTGATGCCACATTTTTATGTGCA
GAACATTGTGATGACTTCTGAATTGAAAGCTTTGTGGAATAGCTTAATTT
TTTTTTCTGAATATAAAAGTGAAATGCATTCTCATTGCACAAAACTTACA
AACACAGTAAAGTATAAAAGAAAACAAAGAATAGTATGTAAGCCCACATC
TGGATAATCATAGCTAATCTAGCTGATCTTTCCAGAAATAATTTATACAT
ATTTATGTGCACATTTCCCTTTAAAAAATAGGATGATGGTTTACATATGT
TAAAAAATATTTGTTATGAGATTTTTGAACGTATAGGACCTCAAAGTCAA
GTATAATAAACATGAATAAATCTTCCTTCTAGATGCAGTAATAGTTAACA
TTTTGCCATATTTTCTGTGTTGTGTATGTTTGTGTATTTATCCATCTGTA
TCTAGAGACATATCTCTAGATACAGGTATCTATATATTTGTGTATATAAC
TATTTTATAATAAATTAGACATCTCAGTACTTTTAGTGCATGTCCAGCAT
GTATTTTCAATAAAAAGAAGATAGTTTTCTTTATAATCATAATATCACTG
TTGTCTTTGACAAAGTTAACATGCTTCTTAATTTAATGAAACAATGTATC
TGACTTTTGATATTGGCAACTGTAAATCCATGTAGCCATTTAAAAAGGCT
ACATAGTAGTCCACTGAATGGATGTACCACATTTTATTTAACTAATTTCT
GGTATACAATCATTATGCCATAGCATGATTTTTAAAAACTACTTATAGTT
CTATATGAAGACCTTTTTTTTTTTTTTGGAGACAGAGTCTCGCTCTGTCA
CCCAGGCTAGAGTGCAGTGGTGTGATCTTGGCTCACTGCAAGCTCTGCCT
CCCTGGTTCACGCCATTCTCCTACCTCAGCCTCCTGAGTAGCTGGGACTA
TAGGCACGCCTGGCTAATTTTTTCTATTTTTAGTAGAGACAGGGTTTCAC
CGTGTTAGCCACGATGGTCTCGATCTCCTGACCTCATGATCTGCCCACCT
CGGCCTCCCAAAGTGCTGGGATTACAGGCATGAGCCACTGTGCCCGGCCA
TGAAGACCTTTTTTAAAAGCTTGGTATTGGTCCTGTTCACAGAACTGTAA
CAAAAACTTCTACCTTTTGAGCCAGAAGCAGCTTAAATGTAACATCATAA
TGTGTGACAGTATCAACATTAGAGCAAATTTTTTTAAAGCACTATATAGA
GTCAACATAATTTGTATGTCTACTGAAAATTTGATCACATATGTTTAAAA
AAAGAACTGTGGTTATCTCTGTTGGAAGATGTTTATAGGCTTCTGAAAAA
ATATAGTTTCTGTATACTTTAGACAGTTCCTTGAGTTCTGCTGAAAAATA
AGGTGACTACCCAGAGCCTTACTTTTAAATTTAGTTATATTTTGGTTCAT
ATTCAGAAGGATTTGAGGACCTAAAAAGAAGGCAGCCACAGTAAGATAAA
TAAAATGGTAATGAAAAAGAAACTGAGTTTTTTGGCATTGTGGTGGTAAA
AAAGAAAACAAATGAGCCAGCAGTAAGGAGAACAAATATTTTTACTGTGA
CTGAACATAAAACACCTCCTAGCTTCTCGGCAACCAAGGGTGAAGGAAAC
ACTAAAAGTTGATAATCTCAGTAGCAGAAGGGAAAGAAACAGTTGGAATA
TTGTCCTTTTTGGCAGTGTTGAAAGTGCTTAATGGTCTATTACACTGACT
CTAGGGAGAAGGGCCAAAGGTGTTTAATCCTAGTATATAATGTTAACCTA
TTACTTAGATACCTAGCCAATGTTCTTACCCCAGTGAAAGGTTTGCATCA
CTTCAGCACAGAAGTGAGTAGCAGGGATAAATGTAGACAAAGCCAGGATT
TTCCCAAGAAACGGTTTTATATTCACTGTAGCCTTAAAGATGTCTTGGCT
GAAAAATGCTCAGAGCAAGCATTTGCAGTGTTGTGTCTCATGCAGTTTTA
GATTGTAAGGTTTTGCAGCATCCCATCAGGATGAATTAGCAGTCTCAGGA
GGTCCTTAGGGAATTGAATGACATTACATACTAAATTGGGGTTATTTGAT
ACTGAGCATTCAGTCCTTGAGGAAAGTCTTGTAAAGGTAGGTTATAGCTT
GATTATGCTTAATTGTTCTTCCTTCTATGAGGTGATAGCAGGAGTCCTAG
TTACAATGAATAAAACACATTGTCAGCATGTAGAAAGTATATTATTCTTT
CCATTCATTGTAGCAGTTCATTTCATTTTTAAGGTGACCAGGGCCTATCA
CACATTTTGTTTTGATCCTTAATATGTATAGACTGTTCATAGCTATACTT
ATGTATTTGTTTGTTCATTCTTTTATTCTAATTCATTCAGTACCCATTTT
GCACTTGGAACTGTGTTAGGCAGAATGAAAATGAAATGATCTTCAGAGAT
CGTATTATAGAATTTATCAAGCAACAGAATATAGAATTTATTCAGCAAGA
AAAGACACCTGTTTTTAGATTTTACCTTGTGGTATGTAATACAGCATGTT
TGCTTCATGTTTTAACATCCATGGTTACGGATTGTACGCGTATTGAAGTG
TTTTAAAGCATTTTGAAATCCTTTCTTTTCACACATTTTTCCACTGGAAT
TTATGAAAATAATTTTATTAAAAGTTAAAATAGCTGTATACATAAAAGAT
ATTAACCTATTTTAATTTCATTAGAAGTGCTTCACATCAAAGTACTCTTG
CGCTATTTCTGCTTGTAGTTTGGCCCCTTCATTGGCCCAAAGATGCCTGA
TGGTGAACACTTCTTATGCCAAATGGCTCACAGCTCTCCCTCTGCAAACG
CCCTCAACAAGACTGAGAATTAGAGGCAGTTCTTGTGCCAAGCCCCACAG
AACCTACCCTTCCTGTACCTTCTTTCTTCACTATAGCATCTCCTGCTTTC
ATTCACTCTTCTTAGAGATCCAGACCTTCCCCTTCATCCTCCTTTTCATT
ACAAGATGGGCAGAATGAAAAACAGCCATTTGTACTTTGAAACATTTTCA
TGAAGTTTGTTAGCTCTACCAGTGGATTTTTAATCCTGTGGTAGTGGAAT
AAAAGATTTGACTTCACCCAGCAAACCTAATGGATAAAATGTCTTTAACT
CTGCTAAATGAATTTCAGTCACAAAGAAAGAAACAGAAGTAATCTTTGGT
GAGTTAAATAGATTGGGCAACAGTGTTTAATGGTATGCCCAAGTTAATCC
TTAGTGTAAACCTGTAGTCATTTTAGTATTCATGGGAGTTGGTGGAAAGG
AAGCTCTGTAATCTCCCATGTGATCTCCCTTGCTCCTCTGGAGAGAAAGT
CTAGTGGTCCAGGGGAATGCAAAGGGTATTGAATCAGATGAAGATCTGAT
ATTTTTCTCCGTGTACTTGCTCTTTGTCCTTGGCAGGTTACCTAAACTTT
CTCTCCCAGCGTGGATTTCCTTGCATATAAAGTGGAACTCCTAATCGCTG
CCACTTGAGCGGTTATAGAGAGGTTGCCTCTTAGAGGTTTACTGATCTTT
AAGTCCTCATTTTCCCACCTCCTCAGGGTCAGTGCTTTCCATGTTAGTAA
CTCCTGCAAATATGAGGGGAGCGCACAGCTTTGCCACTTGTGCATTGGTG
ATTGTGCCACGGTTTTATAACTGGGGTTTAAAAAGTGATACATGCTCAAT
TAAGACAATTTTTAGGAAATACAGAAAACAAAGCTGAAATTATCTATAGT
CTTACAACTTAAAGACAACTCTTAACTCTTTGCATATTTCCATGCAGACC
TTTATTATTTTTCTCCATTTTTATAATTATCTTGCAAATGGGAATTTTTT
ATCTTACTTTCTTGTAAGATAAGCTTTTACTTATATTCTCATATTGTGAC
CAATTGTGACTTATTATCCTGTTATTCTTTCCTTATTAAACATCCTTAGA
AGCAGGATTTGCCAAGTTAAGGAGTAGGAATGTTTTAAGGCTATTTGTTA
CTGTTATTTGTTATTAAATGTTTGTTTTATATAGCATAGATTACGAAGTG
TTGGGTATAAAAATTGAGTTTACCTTAGTTTTTGTCTTCTAGCAATTTTT
AGCTTAGTGGGGAAGATGATATAATTATGTAAAAAGTTACATAACCTTGG
GAAAAATTAGCAGTTATTTCCAGATGTCAGTGGAGGAGGTGTTATTAGAC
AGTAGGATGAAATGTATGTATGTATGCCATAGAAATTAAAATAGGAACGG
GATTGTCCACTACATGCTTGGTTTTATTTTGCACATCACAGCAACCTTTT
ATGGTTGGTGGGGTCCCTATGTTATAAGAATGGAAACCAAAGCCAAGAGA
AGATGGTCACTCTGATAGTAATTGGTAAATCTAGGATTCAAAAATCTGAT
TCTAAATCCAGTGTGACCAGCTAAGAACTTTTTGGCTTTTGTGGCAGTTT
CAGCCTTTTAGAGACCTTTTTTGATGATCATATTCTTGACCACCGTCTGG
ATTGGGCCTTTTTTTTTTTTCATGTGTGATAATGGAGTAAGTGGGATATT
GTTATCATAATACACTCATGGGCAAGTCAGTTTTTAGATGTCTCTGATTA
ATTGCCCTTTGGAGTAAAGATATAGTGTGGTTAGAGTGTGCCTTCAGAGA
ATCCAGCCCACAGGAAGCAACTTTGAGTTTATGATTTTTCCCTACCAAGA
ATGTGCATATATGGGGATATATGCACATATATAGGGGACACTTAAGTCTG
GTTTAAAATACAGAACACTTATAATTTTACATTTTTAATGGTAATCATGT
TTTTAAGTATTTTGGGGATTTTGTTTTGTTTTAGATTATTTTTATTAGAA
GTAAGCCCCTAACATTTTGGATTCCTTTGTCTTTCTCCCAATTTTTTATT
ATTTTATAGAAATCTTCTTCCTATTACTCATGTGTGTATAGAAGAAGGAG
AAAAGCCCATGGTGATATTGCCTTACATGAATTGGGGGAATCTTAAATTG
TTTTTACGACAGTGCAAGTTAGTAGAGGCCAATAATCCACAGGTGAGAAA
AATTTGCCCAAATACCAATTATTTACCAGTAGTTTCTGATAATGTCTTTG
AAAGTAGATATTAACATTCTTAAATATAAACTGTTTAGAGGCCTGCAAAA
AAGAACCTCAAAATGAACCACAAGATATTTATAACCTGTTGGCTTAAAGG
AAAATGTTGGCTTATGTTCCTGCCTTGAGGCTTACACTGTGCTGATGCTG
AAGTTTTCCTTATATTCTCGTTGTGTACTTATTCATAATCAACTCAAACC
CTTTGTAATGCCATCTTAGGTGTTTTCCTGATAACTGAGGGTACTTGTCC
TAGATCTTCCTCTTAGGAATGGAAACAGACTTTCTAGAGCCATTTGGTTC
ATACTATACCAAAGTCTGGTGATGACTGGAGGTCTAGATTGTGGGAGATA
ATTGGATGTATTGTTAATATGCGTGGCTGTAAGATGGCAGTCATCTAAGA
GTGTTTCTCTTCCCTGCTTGTGTTTATAACTGTAAAGCAAAGGTCAGATA
CCAAGCATATTATGATGGTAGTGGTAGTAAAGCCTAGTGGGTTTTTTTTG
CTGTCAGCTGTATCTTGTTGTCCCATTAAGTTGCTTTAGGATTTATAATA
GTATGCTTATTATAGTTGCCAGGAGAAAGTGGGTTCAAAGGCTGCATTCA
GTATTTGGCCTAATATGTATCATATGCTTTGTAAATTGACAAGACTAAAG
CTGGGATTCTCAATAAGTGGTTAGAGGCTGTGGCTCATCTGCATTACCTG
GGGAAGGATTTTATTCCTCAGCCCCAAGTACTGTCATACACCTAGAAGAA
CACATCAGCAGAAAAAGCTAGGAGCTCGTACTTTGGGTTTGTGTGTGGTT
TTTTTTTGTTTGTTTTTTGTTTTTTGTGTTTTTTTTTGAGATGGAGTCTC
GCTCTGTCGCCCAGGCTAGAGTGCAGTGGCGTGATCTCAGCACTGCCAGC
TCTGCCTCCCAGGTTCACGCCGTTCTCCTGCCTCAGCCTCCCGAGTAGCT
GGGACTACAGGCGCCTGCCACCACGCCCGGCTAATTTTTTGTATTTTTAG
TAGACGGGGTTTCACCGTGGTAGCCAGGATGGTCTCAATCTCCTGACCTC
GTGATCTGCCTGCCTCAGCCTCCCAAAGTGCTGGGATTACAAATGTGAGC
CACCGCACCCAGCTGCAGGGTTTTTTTGTTTTTTGTTTCATTAGACAGGA
TCTCAGTCACCAAGGGTAGAGGGCAGTGGTGTGATCACAGCTCACTGCAG
CCTCAACCTCCCATGCTTAAGCAATCCTCCCACCTCAGCCTCCTGAGTAG
CTGGGACCACAGGAACATGCCACCATACCTGGCTAATTTAAAAAAAAATC
ATTTTGTGTAGAGACAGGGCCTCAGTATGTTGTCCAGGTTGGTCTCGAAC
TCCTGGGCTCAAGTGATCCTCCCACCTCAGCCTTCCCAAGTGCTGGGATT
ATAGGCATGAGCCACTGCACCAGGCTTAGATTTGTGTTAATTTGAAAATA
ATCCCAAAGAGAGAATCTCCTAATAAAAACTACTGTTTGGGGGAAAAAAA
GATTACTGAATTATAAACATTTGAAATATTACCTTGAGAACTCAGCATTC
ACTGAACAGATGTATTGAGCAGCTGCTGTGTGTGCTGGATATTGGGGAGA
TACATCTAACAGATGAAAGGAGACAGAGTAAATGGTAGATTACAGAATAA
TAAGGACTACAGTTGATATAAGTATAGGGATATATGGGAACATAGTCTTG
GTCAAGAAATAGCTTTTAAAAATTCTGTGTGTAGGTTTAAAGCAGTATTT
TTGGATGGAGTAGGATTGTGGAATAAAAAAAGTAAGAAAGTGCCATCATA
TTTGTGTGCTATTTGCAAACCATGTTATTTTTTGTTACCACAGGCAATTT
CTCAGCAAGACCTGGTACACATGGCTATTCAGATTGCCTGTGGAATGAGC
TACCTGGCCAGAAGGGAAGTCATCCACAAAGACCTGGCTGCCAGGAACTG
TGTGTAGGTGCCATGAGCTTGACGCTGTCACTTGATGTAGAGTGCTTCCT
GGATTTGGGGTTCTCTGCGAGTCACAGTGGGACTTGTGTTGTATTTAAGG
GTGGTGATGATCCCTACTTTTTAATACTCTGATTGCATTTGATGTTCATA
TCTTTACTTTTCAGCATTGATGACACACTTCAAGTTAAGATCACAGACAA
TGCCCTCTCCAGAGACTTGTTCCCCATGGACTATCACTGTCTGGGGGACA
ATGAAAACAGGCCAGTTCGTTGGATGGCTCTTGAAAGTCTGGTTAATAAC
GAGTTCTCTAGCGCTAGTGATGTGGTAAGTGCCGGGACCCCCAGATAGCA
AAAGAATAGAGAAACAGCTTTCTGTTTTTAAATGTTCTACTATTTTTATC
TTTTGGGTTTTTAAAAAATCTGCTAGCTGTTCCCGGAAATTTTTATCCAG
AAAGCAAGTGTAATTAGCTGCCAGTCAACCAGTTTATGGAGAGGAGCTGT
GACCCAGACCATAGGCCAGCACTGTTTGTAATCTCATTTCATCCTCACAG
TACTGCTCTGACAGAAGCACTTTTTTTTTTTTTTTTTTTAAGACAGAGTT
CCACTCTTGTAGCCCAGGCTGGAGTGCAATGGCGCAATCCTGGCTCACTG
CAACCTCTGCCTACCAGGTTCAAGCGATTCTCAGGCCTCAGCCTCCCAGG
TAGCTGGGATTACAGGCGTGTGCCACCATGGGCACATGCCCAGCTAATTT
TGTATTTTTAGTAGAGACGGGGTTTCACCATGTTGGCCAGGCTGGTCTTG
AACTCCTGACCTCAGTTGATCCACCTGCCTCCGCCTCCCAAAGTCCTGGG
ATTACAGGTGTGAGCCACCACGCCCATTGACAGAAGCACTATTAACACTA
GGAGATAGGCTGTGGTATCCCAGTTTTAAAGGTGAGGATTTTGAGACTCA
GAGCAGTTACAGTAATTTCATTATTATTTCTTTCTTATATAAGAGGCAGA
CCAAATAGTTGCTAGATCATAAAGATGTTTTCTTTTTTTCAGGTGACCTC
TTCTAAGTGTTATGGCTCATAAAATTTGTAAATCCCCCCGTCATCCTCTT
CCTTTTGTCAAGTTTCTGGGTGGTTTTGCCACTTAGCCAGATCATAGTTG
GGCCCTTTGTGATGTACAGTTTCAGAAGCCACGTTCACTTGTTCATATTT
TACAGCCATGTAAAATTTCTGTCACTTGATTTCTTAGTCTTATTTGGAAG
ACTAAGTGTAATTTATTTTCCTGTCTCTTGCCTGTTAGACTTTATTATTG
TTGCAGAATATTTAGGGATTAGGTAAAACTTCTGCTTATAGGGTCCCACA
TATGTTCACTCAGTGATAATCTGTTTGTGTTATATGTAAAATCCTATAGA
ATAATTTGCTTCTAAGCTTCAACTGTAAGAATTGGGCTTACTGTATCATT
GTATTTTGTGTGTCTATATTTTAAAATTTAGGGCTACTCTTTTGTTTCAA
AGTGTGTATGTTTTAAGGATTAATCTTTTAATCATGTATTACTGTACGCT
AATTAATTATATTAATTGGTATATACTCATTTCCTCGGTGTATGAGTTTT
CTGTGGCTGCTGTAGCAAATTACTACAACCTTGGTGGCTTAAAACAATAG
AAATATAGTCTCTCGTGGTTCACTTCTGAGGCCAAATGTCTGAAATCAAG
GTGTAGTCAGGGCTGGGCTCCTTCCAGAGGCTCTAGTGGAGAAGCAGTCC
TTGCCTCTTTCAGCTCTGGTGGCTGTTGGCATTCTTTGGCTTGCGGTTCC
ATCCCTCTGTTCCAAGTTCATACCACCTTCTTCTTCACATACGTCTTTCA
AACTCTCTTTGCCTCCCTCTTAAAAGGATACATATGATTTAGAACCCACT
TACATAATCCAGAATAAACTTTTCCTCTCAAGATCCTTAATCACGTTTTG
TCATGTAAGGTAATTACAAGTTTCAGGGAGTAGGACATGGGCATATCTTT
GGGGCTACCATTCAGTTCACTGCAGTCAATAATTGAAATGATCTAAGATT
TCTTTACTGAAAAACTGATATTCTAATTTAAAATCCTTAAAATGAGAATA
ATTTGAGAAAGGGGAAAATTCCTTTAATTAACTCTAACTACTAAATAAAT
TTTTTTTCCTTTTTAGAAACTATGTGCTATTTGATATTGATATGGTTTGG
CTATGTCCCCACCCAAAATCTCATCTTGAATTGGAATTCCCATAATCCCC
ACGTCAAGGGCGAGACCAAGGGTGGAGATAATTGGATCATGGGGGGCAGG
TTCCCCCATGCTGTTCTAGTGAGTCTCACGAGATCTGATGCTTTTATCAG
CGTCTGGCATTTCCCCTGCTCACACTCACTCCATCCTGCCACCCTGTGAG
GAAGGTGCCTGCTTCTTCTTTTCCTTCGTCATGATTGTAAGTTTCCTGGG
GCCTCTCCATCATTGCAGAAATAAGTCAATTCAACCTCTCTCCTTTATAA
ATTACTCAGTCTTGGGTATTTATTCATAGCAGTGTGAGAAGGGACTAATA
CAGATATATTGCTCCTTTTACATACCAAGTATTTTGATAAAATGTGTTCA
CTTAATGTGAATGAGCAATTTACTTATATAATTTTTAAATTTTTTTTTTT
TTTGAGACAGAGTCTCGCTCTATCACCAGACTGGAGTGCAGTGGCGCGAT
TGCAGCTCAGTGCAACCTCCGCCTCCTGGGTTCATGCGATTCTCCTGCCT
CAGCCTCCTGAGTAGCTGGGACTATAGGCGCGCACCACCATGCCTAGCTA
ATTTTGTTAAAATCAAGGTCTTTCATTTTATATATCTTCAAAATACTTTG
TTTTAACTCAAGATAAACTATTCTCAAAGAAACAATTTATCTGACATTTG
TGAGATGAACAAAGTAAATATGAATGATTATGAGAAGGGATGTGATCTGC
TTCACAGAGCAGTCTCAGAAGCTAACACTGAATGACAACCTGTTTCATGT
TGTCCACAGCCTCTCCTAGCCAGAGAGGCATCGGGCATGGTGGGTGATGG
GTGGCAGGATGAGGGAAATAAAGCCAGACTCTTGGATACTGAATCCCAGG
GTTCAGCTTATAGAGCAGTGGGGTACTAGTCCTGGTCTGTTTTTCCAGAG
CTCTGATGATAAAGAAAAAATCTCTCTCTAGCATTAATGTCTAGAAGTTA
AACATACCAAGTAAAGCAGTACAAATATGCAAAGCAGGTTATATTCTTTT
TAAATCTCTACGTAATGAGAACCTGGGCTTTTTTTTAGGGAGAGAAAGGT
TACATTTTTGGTTTTGATTTTTATATTTTTCAAATGACATACGAAGGGAA
AGCAGAGTTTGATTTTAGACCAATGCACAGCTGCTGTACTGAATGACAGT
AAGGACTAGTTCTACTCTTTGATAATGTTTTTGGTTTTAGAGTTGCCAAA
TCTTTATTCAGAAATTCTGAATTGTAGCTGGAACTGTTTACCAGCTCTAC
ATTAAGCAAAGCTATTTTCATGGAGTGGGAAAGGGGACAGGAGGAATTGG
TTTGGCTTTGGTTGTAGAACATAATTTTAAAACATAATATCAGTTGAATT
AGAGCAGTGTGATCTCAAATTTACTCCTTTAGTAAAAGCTCACATCTAAA
CATTTTTAAATGTAGAAAAGTGAAGTAAACTTTTCTTAGTGTTTACTTTG
AAAATTTTATTCCGTAGTTGATTTAAAAGATGGATTCACTCGTCTCTTTG
GGATGTTAATTTTTTTATTTCCAGTGGTTGATTTAAAATTTTTATTTTGT
TATTTATTGAAATATAGTTGCCTTTGCTGAAATGATATGGCTGTTGTGGT
GAACCAAAACTAGTTCGATGGGCAGAAAATGGAAGATAATTCTTAGATGA
TAATCTATAAGTATTTCGTGTAATTTGAAGAGGTTGGAAATCTTTTTATT
ATTCTCTTAGAAAAAGGAGCATTGATAGCTTGTATTTCAAAGGGGTATGC
AAGATGGCAAGTAGTGCTATTTGCTTGCACAGTGGAGGCCCCCAAAATAT
GTATTACTTTTTTTTTTTTTCCTTAGCTACTTCTGTTGCCCAGGTGGGAG
TGCAGTGGTACAGTCATAGCTCAGTGTAGCCTTGTACTCCTGGGCTCAAG
TGGTCCTCCTGCCTCAGCCTCCTGAGTAGCTAGGACTACCAGTGTGTGCC
ACCACACGTGGCTAATTCTTTTATTTTTTGTAGAGATGGAGGCTTGCTGT
GTTGCCCAGGCTGATCTTGTACCCCTAAGCTCAAGCGATCCTCCTGCCTT
GACCTCTCAAAGCACTGGGATTACAGGCGGGAGCCACTGCACCCAGCCTG
TATTACTTAATTTAACGTATTTATTTCTGTGTAGGTATACCATTGTATAA
TTAAGCGATGATAGGAATTATTTTTTTGTAACATTTATGATTAAATATTT
TGTAGGACCTACAGAGTGAAAATTAAGTTAGATAATATACTTTCAACAAA
TGAGGCTATAGCTAATAATAAATCTCCAAGCAAGATGCTCCAAGAGCGTT
GAGTCTCTTCAGCTGGGTATTAGCTGGCAAAGTTCATGTTGAGGATACAT
GGTTTGGTTAGGCAGAACATTTCAAATTATATCAGAAATAGTTATTTTTA
TGGTTATGACATTTTGCAGGAGTTCACACATTTTATGTAAGAATCTGCCA
TCATCAGAATGCAGTTACACCAAATACTTAGTTGCTTGTTCATGTGGCAG
CTTTATTTTAAAATATCACCATTAACTTAATGGTGAGCAGATTCACAAGA
GAATTACTTTAGTTAGTTATTTAGTTTGTTATTACAGATAGAGTTTTTTA
TTCATTCTCCTCCTCCGCCTCTTCCTCCTCGTCATCTTTGCCTTTCTCTT
CCACCTTTGTCCAGACAACTTTAGTGGGACCTTTTGCGCCATCAGATTTT
CCTTTAGACTTGCAGTTAGCAACATCCTTCTCATACTTCTCCTTCAGCTT
TGTTGCCTTTGTGATGCAAAGCTGCTTTTTAGTGTCACTTAAATTACTCC
ACCTCTCACCTAGCTTTTTTGCCACATCTCCAATAGAGATGCCAGGGTCT
GAATTCTTGGGGCAGAATTCTGAACAGAACAGGAAGAATCTGGACAGTGA
CCTTTTGGGGACATTGAGGTCCTTTTTGTTTTTTATTTTTTTGTTTTGCC
TCCCTTAGATAGTCCATAATCCTTCATTTCTGAATCATAGCATATTTTAT
CTACTTTTGCCATTTCATCAGATTTAGACTTCTCTTTCCCAGACATTGTC
TTCCACTTCTCAGTGTACTTTTGGACAATTTTGCAAAAATTGACAGGGAT
CTTTGGGTTTTTCTGCTTAGGTTCTCTGTATGCCTGCACAAAGAAGGCAT
AAGCAAACATCTTGCCTTTTGGTTTTGTGGAGTAGCTTTTAGCCACCCTG
ACTGAATTGTTCATGAGTCTGGGCAGCACAGGGCATGACATGTAGCTTAG
TGATTCCCCAGCCTCACGCTAGCTGCCTCCACAAAAGGCACCTCAACAGG
AGAATTACTTTAACATCATAATTACTTTCAGTGTGTTTTGGCATAAACTT
ACTTGAAATAGCTGTGTGACCCAAATTTTCTGAGCTATACTTAGCATGTG
ATGTTAACAATATCCTCCGATTTGCATTTAACGTGGTTAACTCCTAGAAA
GTGCTCACATTTTTCTTTTGTAGCTTATTATAAATTTGTTTCCATATATA
TGTATGTGTGTATATACAATCCCACACACACATATGTATGTATGTATCGC
CTTTGCTTTAGATAATTGGAATTGAAAGGACTAGGAACACCCTTCTGTTT
TCCATGACTATGATGTGACTGTTCGTGTAAAAGTCCTGCCTTTTAGTTTT
TTGGGATTGAATTTTTTTTAATTCTTAAAATATTTTAAAGCACACTTGTT
GAAGATTGCTTTTTAACATTTGGGTTGGAAATGTGTACACACAGTCATTA
TATTCTGTTACCATTTTTTCTTTATGGGTGAAGTTGGGGGCTTAAAAGTT
ACATTTAATATTCTATTTTTGGACACTTCCACCAGAGTCTATACACATGT
GAAATACATATTTTTAGATGTAAATGTAGCACCCAACAAATGATACTGCT
GCCTTGGAGGGAACATTTTCTCGGTCCTTTAAGACATCCTTAACATTTTT
GCATTTCTTCATAATTAAAAATTCTACACGATTTATTTAGAAATACATTG
ATGTTTTCCATCATGAAAATAATACATTGCATCTCTGGGCTGTGTTTTCA
GAATTTTTACTGAAAGGATTCAATATTTCAGTATGTCCAGATTCTTCTGT
TAACGTCAACTTTTAACTGTTGAGCTTAAGAACTATGCACATCAAGATTT
GACTAGTTATAAATGTCTAATATTATTCCAAGATATGACATCATTGTAGG
CATTCTTAAGAATTTCTGGATATTGTTTTTATTGTGTTTTCATCAGATGT
GGATACCACCATAAATTTTTTTATGTGACTTTCTTTTCCTTTATTTGAAA
ATAGAGTTCTTGATTTACATCTTCTTTGAATTACTTAGCTAATTATAGTA
GCTAACGTAATTCTGCTCTTAAGAACTACTTTCTATTAGGAGATTACTAA
ATGTAATCAGGAGTATGCAGTTAGGAATTTCTTTCTTATTGGTTTTCTTG
TCTTTATAGTATTACATTGTGTTTAAGAAGCATTTTCTTAAGAAAAACAA
GTCAAGGAAATAACTTCCATGCCAAATATTATCAGCATGGTTTATGGCAG
TGAAGAGATCCTTTTAAAAGTATTTGAACAGTTTTTAGGGGATAGGACAA
ATCAAAATGTGTTGCTTGTGCCTTTACATTATTTATTTTGTGACTATCAA
TGATCACCTAGAAAATAAAAGGAAGTCTGGAGTTATCCAGAGACTGCATC
TGCAGCACATTGCTACGTATGACTCTTTTAACTCCCTGTTTGCCCAGTGC
TGTGGAGGGAGCCCAGTGCTTGTTGTATCCTGAGCGACCTGAGCGGGAGA
GTCTGACATTTATTATATACCCATGGAAAGTAGTCTGCTGAACTACTGTG
GTATCAAAGAGAAAGCTAGAGTTACGGAGGATGCTTCTTCTTATTATTAT
TATTTTATTTTTTATTATACTTTAAGTTCTAAGGTACATGTGCATAACAT
GCAGGTTTGTTACATATGTATACATGTGCCATGTTGGTGTGCTGCACCCA
TTAACTCATCATTCACATTAGGTATATCTCCTAATGCTATCCCTCCCCCA
TCCCCCTACCCTACGAAGGCCCCGGTGTGTGATGTTCCCCTTCCTGTGTC
CAAGTGTTCTCATTGTTCAATTCCCACCTATGAGTGTGAACATGCGGTGT
TTGGTTTTTTGTCCTTGCGATAGTTTGCTGAGAATGATGGTTTCTAGTTT
CGTCCATGTCCCTACAAAGGACATGAACTCATCCTTTTTTATGGCTGCAT
AGCATTCCATGGTGTATATGTGCCACATTTTCTTAATCCAGTCTATCATT
GATGGACATTTGGGTTGGTTCCAAGTCTTTGCTGTCGTGAATAGTGCCTC
AATAAACATACGTGTGCATGTGTCTTTATAGCAGCATGATTTATACTCCT
TTGGGTAGATACCCAGTAATGGGATGGCTGGGTCAAATGGTATTTCTAGT
TCTAGATCCTTGAGGAATCACCACACTGTCTTCAACAATGGTTGAACTAG
TTTACAGTCCCACCAACAGTGTAAAAGTGTTTCTATTTCTCCACATCCTC
TCCATCACCTGTTGTTTCCTGACTTTTTAATGATTGCCATTCTAACTGGT
GTGAGATGGTATCTCATTGTGGTTTTGATTTGCATTTCTCTGATGGCCAG
TGATGATGAGCATTTTTTCATGTGTCTTTTGGCTGCATAAATGTCTTCTT
TTGAGAAGTATCTGTTCATATCCTTTGCCCACTCTTTGATGGGGTTGTTT
TTTTCTTGTAAATTTGTTTGAGTTCTTTGTAGATTCTGGATATTAGCCCT
TTGTCAGATGAGTAGATTACAAAAATTTTCTCCCATTTTGTAGGTTGCCT
GTTCACTCTGACGGTAGCTTCTTTCGCTGTGCAGAAGCTCTTTAGTTTAA
TTAGATCCCATTTGTCAATTTTGGCTTTTGTTGCCATTGCTTTTGGTGTT
TTAGACGTGAAGTCCTTGCTCATGCCTATGTCCTGAATGGTATTGCCTAG
GTTTTCTTCTATTGTTTTTATGGTTTTAGGTCTGACATTTAAGTCTTTAA
TCCATCTTGAATTAATGTTTGTATAAGGTGTAAGGAAGGGATCCAGTTTC
ACCTTTCTACATATGGCTATCCAGTTTCACCTTTCTACATATGGCTAGCC
AGTTTTCCCAGCACCATTTATTAAATAGGGAATCCTTTCCCCATTTCTTG
TTTTTGTCAGGTTTGTCAAAGATCAGATAGTTGTAGATGTGTAGTATTAT
TTTTGAGGGCTCTATTCTGTTCCATTGGTCTATATCTCTGTTTTGGTACC
AGTACCATGCTGTTTTGGTTACTGTAGCCTTGTAGTATAGTTTGAAGTCA
GGTAGCATGATACCTCCAGCTTTGTTCTTCTGGCTTAGGATTGTCTTGGC
AATGCGGGCTCTTTTTTTGGTTCCATATGAACTTTAAAGTAGTTTTTGAA
TCTGTAAATTACCTTGGGCAGTATGGCCATTTTCACGATATTGATTGTTC
CTGTCCATGAGCATGGAGCGCTTATTATTTTTTAAAGTATAGAAAACTTT
ATTCATTATTAATTTTAGAGTAATGCATAGATTCCTAATATTTGTTTCCT
TTATAGTTGTAGATTTTATATACAATAATCTTGGAACAATTATTTAAGTA
TTAAGTTCACATTAAGAGAAATAAATATTAAAGACAGGAAAAATCTAGAG
GTGCAAGGATTATTTCAGACTTTAAGAAATAAAAATTTAAAATTTAACAT
TAAGATATTGTTTAGTTTTATTTAATGGTATTGTTTTATTCATCCTAATA
TTTTCTTGGAATGAGGAATAAATGGATTTACTTTAGGAAGAGTTACTGTA
TTTTCAGAGGTGAGCACTTGCTGTCCTGTGATCAAGTAGTTGGCCTGGGT
GTTTTCTGTCGTTGGCTCTCCCTCAGGATAATTTACTGATAAAATCTTGT
AAACAACTCTTTACATTCCTTTAAAGAGACTTGTCCTTATTCTGTTTAAT
ATTCCCCTCAGATAGCCCCAGTGTTCTGATGGAGTAATGTCTATGCATCT
GATACAGTGGCAAAAAGTGCCATAAATAACAGATATGTGTAATGGCCTGG
TTAGCATCCAATGTTGGCTCCTCTGTCTTTCCTAATCACAGATCGTGGGG
CAGATTCATAGCAGAAGTAGTCACTTTTCAATCACGTATAACACTCTGCT
TAAAGATTGCTGATGTTTGAAGTTGAGTTGGTGTCTTAGTGTATTGGAGC
CACTGTAACAAACTGCCATAGACTGGATAACTTAAATAACAAACAATGAA
TTCTCATAATTCTGGAGTCTAGGAAGTCCAAAATTAGGTACCAGCAGGTT
CGGTGTCTGTTGAGAGCCCCTTTCCTGGTTCATAAATATCCATCCTCTCT
TCTGGGTCCTCATGTGGCATAAGGGGTGAGGGAGCTCTCTGGTGGTCTCT
TTTATAAGAGTACCAATCCCATTTATGAGGGCTGCACTCTCATGACCTGA
CCACCTTCCAAAGGCCCCAACTCCTAATACCATCACTTGGGGGTTAAGGT
TTCAGCATATAAATGGTGGCAGGGACACACACATTCAGTCCACCACATTC
TGCCACTGGCCTTCCAAAATTTATGTCATTCTCACATGCAAAATACATTC
ATTCCATCCCAACAGCCCTAAAAGTCTTAAGTTGTTTTAGCATCAGCTCC
AAAGTCTTAAGTCCAAAGTGTCATTTAAATGTCGTCTAAATCTGATATGG
ATGAACCTCAAGGTACAGTTTATGCTGAGGCAAATTCCTCTCTAGCTCTG
AACCTATAAAAGCAAATAAGTTATGTGCTTCAAAAATACAGTGGTGGGAC
AAGTAATGGATAGACATACTCATTCCAAAGGGAAGAAATAGGAAAGAAGA
AAGGAGTAATGCATCTAAAATAAGTTGAAAACCTAACCGAACAAACAATA
TTAAACCTTAGGACTCAAGAATAATTCCCTTTGGCTTCGTACTCTGCTTT
TCAGGTCCACTGGGGTGGGAACATCATCTCTCAGCTAGGTGGAACGTACC
TCAGTGGCTGTTTGCCAGAGCCCCACCCCAGAGGCACTGAGGTCCTTTTT
TGGCACTTGGGGCACACCAGACCTAAAGAGGCATCTAAGAGGAGGCATGT
CCCTCTGGACATCTGCTCTAGGCCTGGTAAAGGGCGTCTACAGAAAAACC
TATAGCTAACATATTTAGTGGTGAAAGGCTGAATTGTTTTTATTTAATAC
CAGGAACAAGACAAGGTTGCCCACTCTTACCACTTCTGTTACTCAGCACT
GTCCTTAAGATTATAGCCAGTGCACTAAGGGAAGAAAAGAGTAGATAGAA
AAAAGTAACTATTTGCAGAATAGTATGATCATTTATGTAGAAAATTCAGT
ATGATATACAAAAAAGCTACTAAAACTAATAAGTGAGTTTAGAAAGATTG
CAAGATCAGTGTACAAAAATAAATTGTATTTTTGAACACTAACAATGAAA
AATAAAGTTTTAAAAAAGTTTATAAGAACATCAAAAATACGAAGTTCTCA
GGAATAAATCTGACAAAAAATGTGAAAGAGCTATACAATACATTGCTGAG
AGAAATTAAATAATACATACACAATAGGAGAGGTACACCCTTTTTGCCTT
GGTCAGAAAACTCAAGATATTCTCTCCAAGTTTGAAAAATATTATCAACA
TTATTAGTCATTCATTAGGGAAATGCAAAATTAAACCACAATGAGATACT
GTTGGTTTGGCTAAAATTGAAAAGTCTGACCATACCAAATATTGACAAGG
ATATGAAAGAACAAGAGCTCTTTTACACTGCTGGCAGGAATGTAAAATGG
GATATTTCCAAACTTTGGGAAACAGCTTGGCAATTTCTTAAAATGTTAAA
TATGTATGTACCATATTCAGTCATTTTGCTCTTAGATAAATGAAAACATG
TTCTTACCAACACTACGCAGATGTTCATAGCAGCTTTATTTTTAATAGCT
AAGGCTGAACATAATTCAAATGCTTATCAACAGGTAAATGGGTAAACTGC
AGTATTCATATAACGAGCTATACTACTAAATAAAAAGGAATGAACTGTTG
GTACATGCTACATCACAGATGAATCTCAGAATCATTCTGAGTAAAAGAAG
CTAGGAAAACAGTATACAGCTTGAATAATTCTATTTATATAATTCTAGAA
AACGCCAACTATCTAGTGACAGAAAGCAAATTAGTGGGTACTTGGGGATG
GTAGTGCTGGTGGTAGGGAGGGGTGGAAGGAAAGGATCAAAACAGAATCA
TTTGGAAGTGATAGATATGCTCCTTATCTTGGTTGTGGCAGTGGTTTCAG
GAGTGTTTATGTATATCAGAACTTATCCAGTGGAATACTTTATGTGCAAT
TTCTTATGTGTCAGCTGTACTACAATAAAAATAAATGAAAACTGTCTTTA
CTGTCTGATCACTGCAGCTAACAAATCTAGGACTACTTGCTAAATTTACC
CAATCTTAAGACCCACCAAGGACTTTTGTTTAAAGGGATGTGATCCAGGC
CTGTCATGGTGGCTCATGCCTATAATTCCAGCACTTTGGGAGGCCACGGC
TGGTGGATCACTTGAAACCAGGAGTTCGAGACCAGCCTGGCCAACATGGT
AAAACCCCATTTCTGCTAAAAATGCAAATAAATAATTAATTAAATAAAAT
AATGTGATCCAGGCTTCAGAATTATTAAGATAGGACAGAGGAACTGGACT
TTTTAGCAAGCACCCCAGATAATTCTTCTCCTTCTTATAAAACACTTAAC
CCAGGTTCATTTAATTGCACTTTGGTAATCTGCAGCCATGTCTTTATGTG
AGAAAGGGATATTAGGTTTCTAATATGAACAGAAAGGCCTAGCCATGGAC
CACCTGATGATGCTTCCACTGTGGTCAGATAAGATAATTTACAATTGAAT
GGAATTATTTTCCTTTCAGTTTATAGTTTACTGAAGTGAGCCTTGCATTG
TAAGTTCCTGAAGTAATCACTGGAAATGTCTTAATTCAGTAGCTCCATCT
TTAGTTAATGCTGCACTAAAAGAAAAAAGGAATTTTACTTACATACTTTT
TGACAAGTCACATTCATATATGTTTTAGTGGAAAAACTGTTTTACATCTT
ATATATAGAAGCTGTAAAATAACATGTTCTGGGGAGAGGAATAAGATGAA
TAGAATAACATTATAATTATCTAAATGTTCTTTAAATGGAGATAGCCACA
ATCTTTCGTAAAACTCTGGAAAGAACAGCATGACCTTCTTATTGCCCCCT
TTCCACCGACGTCAAACAGAAGGAAATTATAGAATAAGTCTGTCTAAATT
TTAAATGGTTCAGTTATCGGAAGACCTTAGAGAGCAAGTTTTCCACAAAT
CATCTCTGCGCCATTTTGTCTTGATTATTTTTTTAAGGGTTGGTTTGCCA
ACATGTAATTAATCACTTTTGTTAGTTTTTAAGGAAATGAAACTGCATTG
TTCCCTTTGGCTTGACTGTTCTTTTTTATGGCGTCTTTTAGTGTTGTAGT
TGTAATATGAAAACCTTAGGTAAATGTTGGGCAGTAATGTGAACAAAACA
GTGGGAAGGGAGTAATTTTTAAAAGCTTATCTACATTGCTGCTGTAATAC
TTACATTCAACTCCAGATCTCAAATACCACCAACTTTAATATCAAAGTCC
CTTAAAGTCAGAAGGTGTTATAATGTCAGTGGATGGGGTTATTCCCTGTG
TACAAAGAGTCAGGATGCACTTGTCACCAATTCCCATGGTTTCTTCTTCC
AAAATCTAGTTTGAATGGATCATTTCATCTCTGGAGGAAGGGATGGGAGC
ACCCTTCCGACCAGTTTCATTTCTTTGGGCTCTGTTCCATCTGTGCAGGG
AACTACCTTCTCTATGGAAATTGGACACGATTTTAAAAATAAAGCATTGC
ATTTACTAATGACTGTCAGTATTAAGATTTCATGTAGATCTTGTTGCTTC
TCAGAAAAAAGTTTCATATACAGTAAGGTTTTAAAGATAGGCAAATAACT
AGTTGTGAGCAGGCACCATCCTTTAACTTTTATTAATTTGAAATCATTTT
TCCATTTTTAAGCAGAATAGAATGTCCCAGTAGGTGTAAGGGAAATTTCA
GTAGCCTCCAAGACTCTGTGGAGATTTTGAAACTATACACGCTTCTCCAT
CTGTCGCCTGCCCCCTCCTCCCAGAGCATAGACTTCAAGGCTGGACTTTG
CTCCATGTTAATGATCACTGTTAATGAAAAAGATAATAGGTTCCAAAAGG
CTGACCACCTGTTTTTGGTATTTAGGGTAATGCTGCTGTCAAAGTGAATG
AGATATATTCCTTCCTCTGTGTTCTGAAAGTTTACGTAGGGTTGGGTGTG
TAATGGGTTGAATTGTGTCCTGCGTCTCCACCAAAAAAAAAAAAAAAATG
GTTGAAGCCCTAACCCCCAGTACCTCAGAATGTGACCTTATTTGGAAATA
GGGTCATTGCAGATGTAACGTTAAGATGAGGTCATACTCAATGAGTATGA
GCTCTAATCCACCCTAAGAAAAGAGACACACACATGAGGAGAAAATGTCA
CGTGAAGATAGAGGCAGGGACTGCAGGGCTGCAGCTGCAAACCAAGACAC
ACCAGGGGCTTCCGGCAACCACAGGAAGCTGGGAATAGGCAAGGAAAGAT
TCCCCCTACAGGTTTCCGGGGAAGCACAGCCACGCTGACACATTGATTTG
GACTTCTAGCTATCAGAGCAGTGGGACAATAAATTTCTGTTGTTTAAACT
ACTCGTTTGTGGTATGTTATTATGGTAGCCCTAGAAAACTAATACAGATT
TATATTTTCTTTCAAGTTTGATAGAATTCACCAAACAGTGTTGAGTTTTC
TTTGTGGAAAGGTTTTGAATTGTGAATTCAGTATCTTTAATAGATGTAGG
GCTATATAGATGTTCTGTTTACTCCTGTGTCTGTTTTGGTAATTTGTATT
TTAAGAAATTTGTTTATTTCATTTAAGTTGCTGAATTTATTGGCATAAAA
TTGGATTGTTCATAATATTTTTTGATGTCTTAAAGGATGGTGTTCCCTCC
TTTATTTCTGATATTTTAAATTTTTTGTTGTTGTTCAGTCTAACACAGTG
GTTCTTAACTGGGACAGTTTTGCTCTGGCTAAGGACATTTGTCTGTGCCA
AGGGACATTTGGCTATGTCCGGAGGCATTTTGGGTTGTCACAGCTTGGAG
GATGTGCTACTATCATCTAGTGGGTAGAGCCCAGGGAGAATGCTAAACAT
CCTACAATGCACAGGACAGCTTCTCACAGCAAAGAATCCTCCAGCCCACA
ATATCAGTAGTGCTGAGGTTAAAAAACCTACTCTAGGCTTTATAGTGTAA
ACCTTCTATTTTCCAAAAATATTATATCATTTCATGTTTAATATAGTGTT
TCCATTCATCCCCTTCCAGTCTTCAAACTGGTTGTGGGCATACCTTTTAC
TTCTCTGTATATTATAAACCCCACGATATATTGTTAGCTGTTTTTGCTTT
AAATAGTCTGTCAAGGAAATTTTAACATAAGAGAATATGTTCAGTTGTTA
CTAATATATTTTCAGCACTCTGTATTCTTTGTGTTGACTCAGATTTCCAT
GTGGTATATTTTCTTTCCACATGAAGAACTGCTTTCATACATCTTACAGG
TCTGCTGGAAACATAGTCTCTCTGCTTTTATTTGTCTGAAAAACTCTGTA
TTTATCTTCATTGTTGAAGTATATTTTGGCTCTATATAGAACTTTCCATG
ACAGTTTTTAATTTTTTTTTCTTTCTTTGAGACAGAGTCTCGCTTTGTTG
CCCAGGCTGGAGTGCAGTGGCGCGATCTCGGCTCACCGCAACCTCCGCCT
CCCGGGTTTAAGCGATTCTCCTGCTTCAGCCTCCCGAGTAGCTGGGATTA
CAGGCATGCGCCACCATGCCCAGATAATTTTGTATTTTTAGTAGAGATGG
GGTTTCTCTATGTTGGTCATGGCTGATCTTGAACTCCTGACCTCAGATAA
TCCTCCCAAAGTGCTGGGATTACAGGTGTGAGCCACCGTGCCTGGCCCTA
AAATATTTTAAAATATGTCATTTCATTGTTTTCTTGTTTGCATAGATTCT
GGGGGGAAATGTAGCTATTGTTCACTATTATAAGGCTTTTAAAAAAATCT
GTGTATTTTACAAAGTATTTTGGGATTTTGTTGTTATTGAAAAATAATAA
CCTAAAATTTAATGCAACATGTTTAGATTTTACAAACAGACTTGTATTTG
ACTTGCGTTTGGTATTTCTCTTTTTTCCTTTTCTTTTTTTCACTTTTTTC
TCTTTTACTCATTAAAATATGTATTTTGGGGCAAGCTATTTCTCTGAGCC
TCAGTTTTCTCACCTGTAAAATTGAGCTACCAATGATTCTCACAGGTTTA
TTGAGGAAATTAAATGTATTGAAGGATGCAAAATGATTTGAATTTGTAGG
TACTCATAAACCTGATACTACAGTACACAGGTATCCCCCTCCTCATCCAA
GTTCAGTTGTTCTGACCTCAGGGTTCACACTTGGCGGCAGATCAGTGGAT
GTACCGGATCTGAGTCCTTCCCAGCCATGTGGCAGCAGCTCTGTGTAGAG
ACGTTGCCTGCCCCCCGCCCCCTGCACGCTCTCCAGGAGCCAAGTACAGC
CAGGAATTGGCCAAAATGGAGAGCCCCGAACGGACCATGAATGGACACTG
TTGCCACTGTTTACTATTCCTGTTCCTTTCTGTAGTTTACCTGCTGATTT
TAAATAAACCTGATTGCTTACTCAAGCAAGGTTGGAAGTTTTTTTAATCC
TTTAATTTAAAACTTTAAATACAGTAAAAATCCATGACTTCTTATATTTC
CCCTCATTTGCGTATTTCACTTAGGATGCTCTTCCCTTTAATTAAACTAA
AATTCTGTTCATTTTCCACTTCTCAATTATGCAGACTAGCTATAATATTT
AAAGTTACAAATTCTCATATTAGAAGAATCTTGGACATAATTCATAATAC
GTAGCTTGTAATCAACAGTAAACTATTACTTCCAATGGATCTAATTACTT
AGAATTTATAATACTAATATTGGATGCACTCTTTTTTATCTTAGCAAGGG
AAACTAACTGTGTATGATAGACATTTTTTTGTAAATAACATTTCGCTTTT
CCAAAGCATGAGTTGAGCTGTTATTTTTTGTCCTGTAGCTGGCTGTGGGC
GCTAGCCCCCTGACTGTTGTTTTAAATGTCCCCTCATTGTGCTTCTGCTC
CTTTACTAGTGGGCCTTTGGAGTGACGCTGTGGGAACTCATGACTCTGGG
CCAGACTCCCTACGTGGACATTGACCCCTTCGAGATGGCCGCATACCTGA
AAGATGGTTACCGAATAGCCCAGCCAATCAACTGTCCTGATGAATTGTGA
GTTTTTTTGAGCTTGAAGGCATGAGTTTTATTCCATTTACTATATTGGAA
TAAAGGAAAAAGAGCAGGTTTCCATATTCACACAGAGCCTTCCCTTTTGA
GAATAATGACTCTTTGGATTTAAATTAGACTCTTATAAAAAACCACAAAG
TCAGGGAGATTCGTTCACTCTTTCATGCATTTGATATTAGAACGTCTTGC
ATTTTATTTTATAGTGCATAATCTAAGCACTATAAATTAGTCTGAGAGGA
CGTACTAAGAAAATTGAATGCTACGTCAGTTATTCTATATAAAGTGAGTA
TCTGACTTTTATAGTTATAAAGTAGTTATAAAGGACATAGTAGTTATAAA
AAACCTCTCTCGTTGTCATTTTGCCTTGCCAGTTAGTATCAGCTAATACA
GGGAAGATGATCTGGACTAGTAAGCCAAATAGTTGCTTTCTGTTTCCTGC
TTCTATCACATAGCTTATCTTGCCATGTAATGAGAATGGGATAAACAGGA
GGCTATGCAGGGTGATTGATCGTACAGATGAGTATATTCTCAGTTCCAGA
ACCTCCTGAAGGTTTCTGCACCATGACAAGGTGGCAGCTTTTGTACTGAT
TTTACTCTAGATCCAGGGTTCATAGAACTTAGATAAAACTTTCTCTTCCT
CCAAGTTCTTTTCTTAATTTAACTACAGGAAAACACACATTCTTTTTTTA
CTGTGTTTTAGGTTTTTACATGAAATCTCTGTATTTGCTGAAATATTTTG
ATATGTTGTCATGAAGAATTGAAATTCACACATAATCCAAATAATTTCTT
TTAGCTTCTTTCTAGAGCTGCCAACAGATAGTAAAATGGATCCGTTTTTA
ATATATCATCCTCTCCATACCCACCTTTAGACAGGCAACATAACTGGCAT
CAAACTGAGCAAAAGTTATGAATACTGTATCCTTACTAGCCCAAATTTTC
ATTTTTGTCACTCTGCAGATTTGCTGTGATGGCCTGTTGCTGGGCCTTAG
ATCCAGAGGAGAGGCCCAAGTTTCAGCAGCTGGTACAGTGCCTAACAGAG
TTTCATGCAGCCCTGGGGGCCTACGTCTGACTCCTCTCCAATCCCACACC
ATCAGGAAGAAGGTGCCTGTCGGGGCTCACTTGAAGCCTGTCAGGGATGC
TTTGTATCTAACACAACGCCAACAGAAGCACATTTGTCTTCCAGAACACC
GTGCCTTAGAAATGCTTTAGAATCTGAACTTTTTAAGACAGACTTAATAA
TGTGGCATATTTTCTAGATATCACTTTTATTAGGTTGAACTGAAAGGGTT
TTTGTAAATTTTTTGGCCAAAATTTTTTAAAACATACTTACTTTGGACTA
GGGGTACATTCTTACAAAATAAATAAACAGTTTTTAAAATTGTTTAGACA
CAGATATTTGGAATTAGCTATCTTAGTGCCAACTGCTTTTTATTTTTTTA
CTTCATCAAGGTGATGTAAGTGACTCACCTTTAAAGTTTTTTTAGTGTTA
TTTTTTATCACTACTCTGGGAAATGGTTTGTCTTCAAGATGCAATACTTT
TCTTAGTAAAGGAAAAACAGCATAAAAAGATACCTGGTCTGCCTTGTACA
AGAAAAGGCAATATTAGAGGAAGAAAATTTAAAGAAAAGCTAGAGGAAAA
AAAAATTTTTTTAAAAATACTTATTAGAAGCAAACTGCCCTTGCATGGAA
AACTGTTTATTTTTTTCAGTGAAAAGGAATTCTGCTTTCGTGTTTTTGGG
AAAGCAGGAACTGAGTTCATTACATCTTTAATTTGGCAGAAATTAGCCTT
TCTGTGAACCAGATGTGGTTTGGGGCAGATCTGTAGTAAACAATGGTGAT
TTTATTTATTTTTACTCTCTGGAAAAGGAGATAATACAATTCCAGAAAGT
GAACTCATATTTCTAAGGTTAAGATTCCCTTTTATTGCACCTAGAATAGT
GCTATGCACAGAGCGGGTGCTTGAGTTGTTGTCGTTTTTTGTTTGTTTTT
TAAATGTAAACTGGTAAATTTTGTGCTTATCTTCAAGGCTGGCTTAAGTA
TAAAATTGTTTTTTAAACACTTGAAAAATTAAAGGATTTGTTTTATATTA
TGACAGTATTGAAATTATTTTTCATAATGAATGATTGGTTATTGTGTCTG
GTAAGTCTTTGAACATTCAACAGCCAGACATTTGTGTTTTATTTCATGAT
GTTCCAGTCAAGTTCCAAAGCCCTAACACAGTTAAGCTGGCTCAGACTCC
AGGTTCTAGTAAAAAGTTGGAATTAATGTTATAAGGAAGTATTAAAACAC
TGAAACATTTCTCCAGAACCAGCAAGTAAGGGATATGTATGTATTTATGC
TCAGTTTTAGTTGGCCTAAAGCAGAGTTGAATGGGCTTTCTAAATAGCTA
GGCCTGCAGGTACCTGCCACTACTCCCATCTTCAGAGGTATATAAGGGAG
AATGTGTAGCAGTTGGAGGCTTTTGCTGTTTTTAAAAAAGCCTTATGAAT
CAGCAGCACATCGGGAAAAATAGCTCACATAGTACCTGGTTTTCCACAAG
TAAGCCAAGGGCATGATTTTCTGTGTACATTTATTAACAGTTCTTTGGTT
TTATGAAATACTCATATGAAGCCAGTCCCTGGAGTACTGTTTTTTAAAAG
GTCCCTTTGAACCATTTGTAAATTATATTTTCATTCATAACCTGCATTCT
TAGAAGGCATTCAGTCAACATTTACAGCACTTACTGTGTATTTTCCACAT
GGAGTGGTTCAACTCAAGCGTCCCTTCCAGTATTCAGGGCATTCTTATTT
CATGTTCAAGTGAGTGCATTGTTTAGAAATCACAGTTTATTAACATGTAC
ATGATCTATTTTAAAAAAAAAATAAGTACTGACACCTGACTTCTACTCCC
AATTAGCAGATTGTAAGAAATGTCCTCAGTTTGGTGGATGGTGTGGTGCC
TGTAACCTTATCTTTTTGTGTTTGTGAGCACTTTTATTTTATGTATGTAT
TTTTATTGTAGTAGATTTTTCTTGGTGAGCTACTTGGAGTCTTTGACATG
GGAAGGAAAGAGCAGTTATAAGATCTCCCTGACTCATGTTCTTCCAGTAG
TTTTATTGTAGATTGTGAATGTTACTGAAGTTTTACATTTTACTGATTTT
GCAGAAAATATAATTTTTATGGAAAAGAAGCAAATTGACAAAGCAGTGTT
GTGGTATTATAAGCTGGCTCTGGTGACCTAGAGATGCATTCTATTCTGTT
TTTTCTTGTAATCCTCCTGTTCTAGAAATTTTTTTTTTTTTTATTGATAG
AGGGAGGATTGGGTTGCTCATTCAGTATATATTTATTGAGTGCCCACTGT
ATCCTGGCTCTCGGGATAATGCAGGGAACAAGATAGATGAGGTTCTTCCC
TTTCTAGATTTTGCATTCTAGTTAAGAAAAACAGGTAATGAACATAAATA
AGATTTTGGTAAGTGCTATGATAGAAATAATAGAGTGATATTAGAGAGTG
TAACTTTAGATGTCATAATTAGCCAAGCTTCTCTAAGCAGGTGGCATTTG
AATTGTGATCTGAATGGCAAGAAGCAGCCAGCCATGAGAACACAGAGGCA
GAAGGCACATAATTGCAGAAGCCTTAAAGCAGAAGCAAGCTGGCCTCCAA
TCTCAGCCGTTCCCCTTCATTCTCTAGCTGCTGCTGCTGTTGGAAAAAGA
GAAAGGTAGCTAGCGTGGCTAGAATGTAGTTAGGGAAGGGAGTGGTACAA
AGAAGCAGGTCTTTGTAAGCTGTGGCATACATTTATATTTTTTGCTGAAT
ACAATGGGAATTCATTTGAGGATTTTTCAAGTAAGTAGCGATATGATCTG
ATTTGTCATTTTGAAAAGGTTGTGCCATCTCCTCTGTGAAGACTGGATTG
TGAGAATTCAAAGGAAGAAGTAATGAGACCCTTTAAGAAGCCGTTGCATA
GTTCAGAAAAGAAAAGATAGTGGTTTGGACCACTTTGAGTAGTGAGGAGA
TACAGAGAGAAGGGGCAGATTGGAGATATGTTTTGAAGATACACCAGGAC
CTGGATCCATTGGATGTTGACGGTGGGGTGAAGAAGGAAGCGGGGAGGTG
AGGAATGACTAATTGATTTGGGGCTTGAGCATCTGGTTTTATGGCCAAGC
TATATACTGAGATGGGGAAAAATCTAGGAGAAACTGAATTTTGCATTGGC
CAGAGAAATCCATAATTTGGTGTAGACTGTGTTTGAGATGCCAGTTGATC
ATCTGTATGGAGATTTTTGATAGATGAGAGTCTGCAGCTCAGGGAAGGTC
TCATTGAACATACAAATGTAGGAGTATAGCATATAGACTATTTTTAAAGC
CATGGTACTGGATGGAGTTGATAGGGAGAGAGTGTATAGATGGAGAGGAG
GTCCTAGGACTGAGTCTTGAGCATTCTAACCTTCAGTTCCAGCTAGTGAA
GAAAGAAGAGATAACAAAGACAGGAAAAGCCACTGTAATTGATAAAAGCA
ACAACAAAAAAGTGAAAATGAGGTGTTGCAGGAGCCCAAGAAAGGTCGTG
TTTCAAAATGGAGACGCCTCCATCAGATTTGGCAATATGGAGTTAACTGG
CAGGAGCATGGGGACTGGAGCAAGTCGAAGTGTGGACACAGGTGGGGACG
TAAAGGCCTACCACTGTCAGCCCTGCTGTGAAGGGAAATGAAGCAATAAC
TAGAGGAGATCAAAGGGGGTTTTTCGGGAGAAGAGGGACAGGGATGAGAG
TGTTTTCCAGATGAAGGTACAGTGCCAGAAAATGTTTCATGGGCTCATGG
GAATGATCCGGTAGAGGGAGAAAAATCCCTCTGGGGTATGGGCCACTGCT
CTTCCTTGCCATTTGGTCCACAACCATGAAAGCCTTTCACTCTGGCCCCG
AGCCCTTTTGTTGTATAAGGGTTGTGTGTTTACTGGGGGAGAAATGTGAG
GGAGGTAAGGTAAGGGAGGCGGTACACAAAACTGCCACCGTCCCATTACC
CAACAGCCTGCCTCTGTTGAGGGGCTTTCCTGGGACTTCAGACTGGCACC
CTCATTGTGCTTGCAGCTGTCATGTTTCCTTGCCCAGATTGCCCATAGCT
GCTTCCCTGGCCTCGGCCTTGAACCCCTCTAGCCAGCAACTGCCCAGGTA
ACACTTTATAAAACTGAGCCGCTGTCCCTGCTTGGAGCCTTCAGAGTAGA
CTTCAGACCTAGGCAGAGTATCCACTCAGCTCTGCCGTTTCTCCTGCTCC
CCACTTCGGGCCTGCACGCTTCCCGCCGCTCGGCATGCTCCTTCCCCTTC
TCCGAGAATGACGTGCTCCCCGCACTCCCCACACTGCCGCCTTGGGACCA
AGAAGGTGCCTCTCACACCTTACTGCTCTCCTCCAGGCTTCGTGGTTGCT
TTTCAGCCTTTTTCCCTGGATTTGAACTTTAACAAAGGTAAGATGGAGGT
TTATTTTTCACCACTTGGCTGTGAGTTCCTTGAGGATAGGAACTTGTGTT
TTACTCAGATCTGAATCTCCATTGCTTTAGTGGTGTGCTCAATACATGGT
AGGGGTGAATATTTAATGAATGAATGAGTGAAAATATCTAAATTTTGCAC
AGGAGAGCAATTAGAGAGGCTTAAACAGGAAGGAAGTCTTCTAATTCCTT
AAAAGCATTATCTCATATTTGAAGAGTTCAAGTTTGATGCAACATAAACT
GATAAAGTTTGAAATAAAAAGAGACAGGTTGGTAGGAAAGACCATTCATA
TCCTATCCCCAAACTGGCTTAAGTCCACTCCCACTGCCCCCAGCTACCAC
CTTTTTACTTTATTCTACCTGCTATTTCTTTGGCCACCGGAATAATAAGC
CTGATGTAAATTCTGTTTCATACTCCCACAGGTCAACTTTTTTTGGAGTT
TGACAATAATTATTCCAAGTCAAGTAATTCATTGATTTTAGTGGAAGATT
GTTTTCCAGGTGTTATTCTTCCATGCGCCTCACCCCCATCTCATAAAGTA
GAAAAGAGATGATTTAATTTATGGGTCTAGAAAATAAAAATGTAAATACT
TGCTTGTTAAATTAATTTTTTTCTACCTTCATTTTTACCTTTAATATTTC
AAGATAATATTGGACTTAATTCTCTGAGACACACAAACATCCTGCATAAG
TATATTGACAACATACATTTTATATGTAAGTGAACCATATGGGTGAGCCT
CTGCTTTCTCCCTGACAGCGCTGTTCTTTAAGAAATTAAGGCTAACCAGG
TCTATAGGTTTTAATGGGAGATGTCTCCTAACTTTTCAAAGACCAGATGT
TCTTGTTATACTGATAAAAACTTATTAAAAAAAAAAAGAAATAGGGATTG
ATGATGTTCAACATTATTCTCTTTGAACAAATACATCATTTGTTCATACT
TGGTTTCTCAGTATTTCAACAATAAAGCATGTTTTAATGATTTGTCTTTA
GAGGAGTGAGCACTCCTCTGGGGGTATATTACAACTGGGAAAAAAAATAT
AGTTTGAAAATAAAATAACTGTTTACCCTTAAAGAAATTAACTCTATGGT
GCCTTAAGGTTACATTTGCAATAAACTTTTTTACATCTTTCTGAGTCTTT
TTTTGCTTTTTGCCTTCTTGGCTCTGAGAATAAGCATAGGCTGATATTAG
ATAGGTTTGTATCATTAGGGATAGCATTTAGCTGCAAGTAATGGAAAAAT
TCAACCATAATCACTTAACCAAAGGCAGGTCATTTTTCTCCCATAACAGC
TCCAGAGAGAGATAAACCAATGCTAATGCAGTGATCCCAGGGTGTCATCC
ATTGCCAGTCTCCTCCTGTTTTGCTGCTCTGCCGTCTTTAGCATGCTGTG
GTTCTTAGGGACCCCAAGGGCCTAGAGCTTGTTCCAGATAGGAAGATGAG
CAAGGGCAAAAAGCAAGAGGCATGAGTTACTGAGTCCTTTTTGTGAGGAA
ACTACTAACTTCCTGTAGTATGCTTCCACCAACAACTTAATATCCTGAAC
GCACATGAGCACTCCATGCGCTACAGGTGGGTAATGCCAGGAAATTAGAT
TTTGTTATTTTTTGAAAACAGCTTTATTGAGATATAATTCTAGTATCACC
CATTTAAAGCGTGTAATTCTCAGTGATTTTTAGTGTAGTCACAGAGTTGT
ATAATCATCGCCACAATCTAATTCATTTTTATTACCCTAAAAAGAAACCC
CATACCCATTAGCAGTCACTCCCAATTCCACCCTCACCCTCAGCTCTAGG
CAACCACGAATCTACTTTTTGTCTGTATAGATTTGCCTGTTCTGGACCAT
TCATATAAACGGAATCACACAACATGTGGTCTTTTGTGTTTGGCTTCTTA
CATTTAACATAGGATTTTCAAAATTCCTTCATGTATCATGTATCAGTACT
TCATTCTTTTTTATTACTGACAGATATTCCATTGTATGGATATAAACCAT
TCATCAGTTAATGCATATTGGAGGAAATTGAGTTTTGAGTTGATCACGTT
CTTGTTGTATACAAAATTGGGCTTCTATTGGTAAGGAAGGCGATGGCTCT
TGAGGAACGGCTAGCAGTGTCTGCCGCTTTGCAGAAATGTGCTGGTATTT
GGCTTCCTTTAGCAATATATTTAATGCTTCAAAAGTTCTGTCAAAGTACA
GTGATTGTGAAGAGTCTTAGTGACTGTAGGGAGTGTCCAGTCAGCTTGGT
GTTTAAGTTGGTGAGTCCTGCCATTATTTGAGATGAAAGCCTGGTAGGAA
TTACTACCTCACCTTTAGCTTCTAAGTCTTTGACCAGAAAACTCTACTGC
TGTATTCCATTGTGAATGCTGTTACTTTGGCCTCTGGCCCACTGTTTTCC
ACCTGTTCATAATTCTGCCACCACCTTATAGATTTGAGCTTGCAAAGTTA
TATTTTCTTCAATATTTGGCGTACAGTATCTACCACCAGGTACCTTTTTG
CTAGCTTCATGATCTGACTATTTAGGGCCTTTAGACGTAAGCCAGAGACT
GGCTGTGTGTGTCCATTGAAAGTCTTATTAAAAGAGCTTAAAAAATATTT
AATGATCTATTACATAGATTTATTGTACTATATTTCATATGCCTTTATTT
AGACAATACTTGCATTCCTACTAGGTTTTTTGACTTTTTAATTGATACTA
TTTGGTCATAGCATTACTTAATAGGCATCTGAAGAACACTTTAGACATTG
CCTAATTTAGAGAACAGGATACCGTCCTGGAGACTCTATGACGAATTGGT
TGTGGGCCCTCATTCTAGAAAAACACTGGAATTAATATCCTGAAAAAAGT
TCCTCTAGCCCAATGTGGTTCAAGCCAGAATAGGTTTGACTTACCCATAA
TCTCTTTATCAGGAAGTGCAGTGCTTGATAAATCTGTGTTCAAGTTTTGT
GTTGGGATCTCTAAGCAGTGTCCCTCTGGCCTTTGCTCTTCCCCCTGTCC
CAGTCCTCAGAACGGCCCACAGATAACTAAGCTTAAAGGATAGGGGCAGC
TTTATGTGACCTGACAAGCATTCTTGGCCAGCTGAGCAGACTTGTCTTGT
GGTAAGTACTCCCTTAGGGTCCTTCTCACCTCTTCTGGGAAGGCAGGAAG
AGGAAGAGACAGGAGCTTGGCACTGCCGGGCTTCATGGAAACCTGGACTG
GCTCCCACTGAGGGGCTTCCCCTTCTCCATTTTCCTCTGTGTCCACTCTG
GACCTTTTATTCTTTAGTGTCCCTCTGTTGCCAGCCTCACCTTCTGGCTC
TTGTCCTTATGTCCCTCTTAAAGGAACTTCATTCCCTTTCCACAGGGTGT
AGTACTTCAAGTGCATTTAAACAATGTATTAAGACTTAAAGTAGATATGA
AGATTAGTTTTTTCCACAGCCACCTTAGCAGGGCATCACATTTTTGAGAT
GAAAGGAGGATTACCTGTTTTATTTTACAAATAAGGAAACTGAATTCTGT
ATCACTTCCTAGATACGTTGAAAATAGGCCTTTTTTCATGTTGGCATGTT
TTTAGGAGGTAGTTGGAAATTTGTTGAGTACTTACCAGCACTCCAATAGT
GGGCATTTTAATATATGTGATAATAGATTTGTGGAGAGTTTGGTAGTATT
GGAGCATAGTGTTTAAACGAAGAGTAAATGGCAGATGCTGTGAGAGCCTA
ACACGTTAAGACTTTGTAGTAAAAGGCTTTCATCAGGTAGTGATGCAGTC
ACATTTGTGTTAGCAATGCCTACTATACAGCCTTGTACTGATTGCTGTAC
ATGCATGATCTCATTTATAATTTCCATTTTACAGTTGAGGAAATGGAGGT
TGAGATAGATTGCACAGCTCATCTAGGGAGTCTGACCAGAGCACGCACAG
GACACCTGTACAGTTGGGGATGTGTTAGGAGAGCCTACTTGGTGGTCTCA
ACATTTAATGACATAAGAGTGAGAGGTGTTGGATAGGAGTCTGTAAGCAT
CATAAGAGTGTGAAGGTAGGGAGCTGACCAAGAGCAGTGCAAGTGAGGTC
AGCTGAGGTTAGAGACACCTGACTGGTTATGGCATTTTTCTCCGTGAATT
TGGCTGCAATAGAATCTAAATAAGATGAATGTTCTGTCTCTTATATGAGA
GTCCAAAGGGAGGAGAGGTTCATGTTCAGAAACATTCAAGATCCCAGTTC
CCTTCCATCTTGTCCTGATGTGGCTGCATGGCCAATGCTGAGTCTCTACC
ACATCTCAGCTTCTGGGAAAAGCAAGTTAATTCCTTTTAGACAAATAACA
GAGATGTTGTACACATCGTCAAGAATTTAGGCACCTAGATGCAAGGACCT
CTGAGAAAGGAGTCTAGTAAAACTATTACTATGGAAGACGAAGATAATGG
ATTTGAGGGGCCAGCTAAGGCTGGCTGTGGGGTTAATGCAGGATTGGAGT
TTTTCTGGACAGGTGTGTTTTAGGGTGTTGGTGAGATGTAGTTCAGGTGG
ATAGCCATGAGCTCCGGAGGAGATTGAGGACCTGGAACCCTGCAGAGTAG
ATGTGGGACTAAGGGAGAGTCATTTGTGGCTAGAGTAGGATCTGGGTTGA
AGTCCAGAGGTGGCAGATGGAGTGAAGCCATGGTTTGGGAAACCAACCAT
GGTGAGCTGGAGGTTTCAGCATGTTTGGTGTTCCTGCACAGCAGTCACAC
TGCCTACACACATACTCACCACACTCCACTTCGTGTCCATCTGGGTATCT
CCTCCTCACCTACAAAATCTTGATCCCTAGGCTCTTAAAGTCTCCACTCT
GTTTCTCTGGCTATCAGTTTGTAGTTTGTAACTTAAGCTTACATATGGCC
AGTTGTATTAGGGTTCTGGAGATATATATATATCCTTTTATATACATATA
TATATACATATATATATGTTAAGTATTAACTCACATGATCACAAGGTCCC
AGTAGGCCGTCTGCAAGGTCCGAGTCCCAAAACTGAAGAACTTGGAGTCC
AGTGTTCGAGGGCAGGAGATGTAGGATGGGAGGCTAGGCCCATCTAGTCT
AGTCTTTTCACGTTTTTTCTAGCCACGCTGGCAGCTGATTAGATGGTGCC
CACCCACATTAAGGGTGCTGATTCAAATGTTAATCTCCTTTGGCAACACC
CTCACAGACACACCCAGCTATCCAATCAAGTTGACACTCAATATTAACGA
TCACACCAGTTCCGGCCCACCACATATCTGCTGCAACCTGGCAGTTGCTG
TGCTTATTTTCACACCATGATTGGCCCCACTCATCACATCTTTTCACACT
GGACCATAGCTAGCAGGGCAGATGGCTGCTAACCCAGCTGCTCCCCATGT
TGGGTTCAATGCCAAGCAGGCGCAAAGGAGTACAGCACTATCGTTGCTTT
ATCTTTCTGCCAGCCCTAGAGAGATGCAAGTTGGCTGTGTCTCCTTGCCA
CTCAGGAACAGTGAGTTTTGATCAGCTGGTTGAGAATTCAGTCATCGGTC
TGTTCATTAAACTAGAACTGGGAATGAAGAAGCCAAGGAGGCCATCCTGT
CCTCACTGTCTGGTGAGTGGTGGTTTGGGGTATATAACCAGCCAGTTACG
GCACAGTGTATGTGGGAGCCACCCCCAGGGTTTGAGCCCCAGAGAGCCTT
CCCCAAAGGGCTGGTAAATGGGGATCCCGTCCAGAAACACAGGTGATCCC
TGGACGGGAGGCAGCCCCCTGAGCTGCTGCCTCTAGGACAGGAGTGCTGC
TAGAGTTTGTGTGTTAGTCTGTTCATGCAAGTGGCCATCACGTCTGTCCT
TGGGTATGGCTTTCTCAAGAGATTGCATCTCTGAACCTCATACTACCCTG
GTCTCAGGGTGTGACTGAGTGAGCCTCAGCCTGAGAGTAAGGAAATCACT
TCTGGCATCAACTGGATTTTTTTTGCAAAGCCCAGTCTTTTAATCCAAGA
ATCAGTTTTTTGGAAAAGGTGAAAACAAAAAAAAAGCTCCTGTGGAAAAC
TGCATCATACTAGGTCACAGGTTAGTGAGAGAGGTCTGGCCAGTCCTTCA
GATCCAGATGGCTATCAGGGAGATAGGGGGCTGGGACAAACATGGTCCCA
CCAGCATGGGCCTCCTATCAGACCTCGGCCCCGAATCTCTCAGGGAAGTC
CCTGCAGAGACTGAACCGCCTCCATCCTGTCGTTCAGAGGCCCTGTTCAC
ATTCCATCCCCTCTCAGTTCCCTGAGATTTCCCAGGCTTGGAGAAAACAC
ACTCGGATATCACACGCTCAGAAGCCTCTCGTCCAGATGACAACATCCAA
TGCCACTGGTATGACACCACCCCGTACTAAATCCACTACTTATAAATTTG
CCTCCGCATCTCCGTCTCCCTCTGGACCACACAACCCTTCCTCATAGGGC
TGCCTCCAGCCTCCCCACTGTCAGAGCCATCCCATCATGGGGGGATCCAA
GGGCCTGTCTGGGTCATCAGCTTGACTCTCCAGGATAGCTAGAAACTCCC
ACAAGGCTGGGCAGGAAGAAACATCACCTCACTGCCCTGGAAGTTGTGGG
TGAGGGGAGGAAGCCTCGAAGGACTCACTGGGCTGGGGGCTGTCGGGAGT
GTTCCCAGAAAAGCCGCCTGTAGACCTCAAGGTCTCCACGGCTCTAGAAA
AAGGAGGGCTGGCCCCATCTAAGCAGCAGGTAAGGCCAGGGGCCCTCTTG
AGTTTGTGATGTGGTCTCACAGACGCTCATCACTGTCGTCCTTGTCCTCT
CCCTCACCCTCTCACCCTCCTCTCACCCTCACCCTCCTTTTCTGACTCTC
CCCCTGTGCCTCTTTAAAGTCAGCAGCTCCAAATCTATCCAGGGCTCCTA
GTCAGCCTGCCCCTATTCTGTGTCCCTCCTGTGAATACTACTGTCCCCTT
CCACCCTGGATTCCCCACTTTTCCATCTGTGATTTTCCCATTTTTGGTCA
CTCTCTTCTTGTCTACACCCCCTGCCCTCCACTCCCCTGACCTGGGGCCA
GTGCCCTCACTTTCTGGCCAGACTACAGCTACCAGATTCAGTGACCTTTT
CTCAGTCATCCTCCTGACTTAAAACCCTCTCTTCCTTCAACGCTTCTCTC
TTGGGGCTTTTCTTCTTCCACCTCTCTCCCACTCCCCAAAATATCAGGGG
TTTCTAAGGTTCCTGTCTGTCCCTCAGTGACTCCATCTGTGCTCAGATAC
CTGCTCTGTCCCCAGCACCAGGACCTCTGGACATCTCTTTCTGACATTGT
CCCCCTGGATGTCACAGTCCTCTCCCTTCCCGGCCCTCTACCAGGACCAC
CCAGGAAGCCACCTCCAGAGTCATCCAGGGGTAGAGCTTAGGGTCTCTCT
GACAAGCTCTGTCCAGCCCTGCAGGGTTGATGGGGCCAGCCTTTCCTACC
CTCTCCACTCCCTTTCCCTGTTTGGGCCTCCTGTCCCCATTTTCCTCGAT
GCTACACTGCCTGCCAGTTTGAATATCACTCAAGCATTTATTTCAGACAT
TCATAATACTGTCATGAGTGTGGATGCATAAAATCATGTGAAAAAGACTG
GGTTTTTTGTTTGTTTGGTAAGCTCCCCAAGGGAAGACGGACCACTCCAC
CCGCTTCTTTGTGACTTCTAAATATGATGATTTCTGGGAGTGAGAGAGTA
CTTCTACTGACCAAAGATACCCAGAAATATATATATGGAATTCATTATTC
AAAGAGTTCCTACTGACCAAAAGACTTGGAGAAAGATCTGCCAGAGACAG
GGTTGGTTTCTAAGACAAGACATGGACACATATCCTTAATGTTTACATAC
AGGGCGAGATGGCCATGGCCCCATGGATGGAGTGCCTTTCCACCAGCTGG
AGTGCCTTTCCCCCAGCCAGGCTTGTAGTGTTTATTTACTCAACCTACAC
TTACGCATGTACATGATGCTTTTCTAGTGGTGGTAACAACTTCAGCCCAA
TGGCCTTGACCTCTGGAGGCTGGGGTTGGGCTGCAGCCTGTGACCATGAG
CCACTAAGGAAGTAACCCTTACTCTATTTCGCCAGCACGAACATCACCTA
GAGTGTCATTATGAAGGTACCATGAAAGGGAGGTACCAACAACTGTGAAG
GTGATTTCATGACAAATCCACCTTGGGAATTAGGTTCCTTGATTTATTTT
TAAGTTTAAAAAATTTTTACACAGTGCCGTTGTTAAAAATACACTGTCTT
ATCTAGGCACAGGATACGTTTTTGAAGATCTAAGGAAAATATTATTTTAT
AACTAGCACATTGCTACCCTGTTGTCTAAGAGAAAATGTCAAGCTGTGGG
TTTAGAATCAGAGTTTATAACAATAAGAGAGAGAAAACAAATATTTTTGA
AAGCTAATATTGTTCCTCTCATAACCTCTTTTTTAAAAATATTTTTCTTC
TTTTTAACTTTCTGTACTCCAACCTACCTTCTGATTTTTCAGTTTTTTTT
TTCAGTTTTGTCTTTCTTTAAAGTTCAATGTCTATATTTTTCATTACTAA
AAGTTTTCTTTTAAAACATTGCTTTTGTTTTTATAGCATTTATTGTTTCT
TTAATCATTGTAAACAGCTGTTAATTACATTTCTTAAAGTTCTTAAAATT
CTTTTTTTTTTTTTTTTACTCTGTTGCCCAGGCTGGGGTGCAGTGGTG

An exemplary DNA sequence is human Slco2a1 provided below.

gi|224589815: c133763526-133636932 Homo sapiens
chromosome 3, GRCh37.p13 Primary Assembly

(SEQ ID NO: 307)

GAATTTTGGAGGAATCAAGTAGGTATAAAAATCAAATGCTTCCACTTTTG
CTCATGAAAAGATATTTCACTAAATTATTGCAAACTATAAATCCTTTATG
AAAGAAAATTTCCCTGAATCTGAAAAATAGAGCACATAAATAAAGAACTA
ACCATTCTTGTCTCCTTATGTAAGCACTAGGGAAAAAAATCAACTTTATC
AATTATCAACTACTTAATTTCATGTAATTAATTTTGCTGTTGTTTTGCTC
AATTTTGATTAGCAGTTCCAGGAAAAGCAATTTTGGACTATAAATGGTTG
CAAATGCTTCCAGAGAAAAATTGAAAACAATAACTGTGGGTAACAAAAAC
CTAGAACAGCCAAGCTCAAACATCTGACAGAAGTCCACAGTGGACAAGGA
AATTTAGTTATATTTGTGTATATAAAAATTTAACATAATAGCCTAATCAT
GACTGATAGTACATGTCATGACATATCAGAGTTTTAGAACTCTCACACAA
TTTTGGAACATATTTTAATACCACATGTATACAAATATAACTTAAAGGAA
GTTGAACATCATTTCTTAATTGATAATGCTTCCCCTATGATTTCTCAATA
AGCCTAATCATTTAATCTTTATGCAAGATGAGAGATACATTCTTTGAGGC
TCTCCAGGAGCCCACGTGGAAAATCCCAAAGTTAATTTTAGGCCAAAAAG
ATTTAATTTTGGATTTTGATGCAGGGGAAATCTACCAAAGATGTCAAAAA
GTCTAAGGTACTTGATTGAACAGAATCATAGGTCACTGCTAAATAATAGT
TATTCATTTAACTAGAGCGGTAATCAAAAGACTTCAAAAGCACTACAGAA
AGTTACATGGATGTAAAAACCTTAATCCTTTTAAAGTTCAATTTTCCTAA
ATATCAAAAACTCAATAAAGATAACATGAAACACAGGAAATTATCTTGGT
AAAGCATAGAATCTTTGTTTCCTAGGCCAGTTACCTAAAAGGCAAAGAAA
AAAGAAAACCGCCTGTAGTGTGATTGCTTTTATTTATACAATGTTCCTTT
AGATAACTTGTAAGTTAAGCCTAACAAAAAGAGGACTTGAATTTAATCAG
GCACAGGATAAGTTCATCCAGGGTTATGAATGTACTTTATGTTATAGAGA
AATGTAAGCAAGAAAACTGGCACCTTGCACAGGAGAATGTCTGGCTTTTA
GTAATGACATAGGAAATTTCCTGGTTACATGAAACAATTAAGACTTATCA
GGAAAAGTCAAAAGTACAGAATCAGGCCGGGCATGGCAGCTCATGCCTAT
AATCGCAGCACTTTGGAAGGCCAAGGCAGGTGGATCACCTGAGGTCAGGA
GTTCAAGACCAGCCTCTCCAACATGGTGAAACTCCATCTCTACTAACAGT
ACAAAAATTAGCTAGGCATGGTGGCAGGCACCTGTAATACCAGCTACTCA
GGAGGCTGAGGCATGAGAATTGCTTGAACCTGGGAGGCAGAGGTTGCAGT
GAGCCAAGATCACACCACTGCATTCCAGCCTGGGCAGCAGAGCGAGTCTG
CATCTCAAAAAAAAGTACAGAATCAAGTTGTACTGTAGGAAACACTGCTT
TCCTAGACCTTCAAGATAAACACTTATACAAAAATTAGCTGGGCATGGTG
GCATATGCCTATAATGCCAGCTACTCAGGAGGCTGAGGCAGGAGAATCAC
TTGAACTCAGGAGGTGAAGGTTGCAGTGAGCCAAGATTGCGCCACTGCAC
TCCAGCCTGGGTGACAGAGCAAGTCTCTGTCTCAAAAAAAAAAAAAAGAT
GAACACTTTAGTGACAAATTGTAATGGCTGTGCTAAAACCAGGGAAAAAG
TTACAGGACTTGGCAAAAATGTTGAAGGAGAGGGTAATCAACCCAGCCAA
GCAAAAAGATAGACCTTTTCAAGGGTAGAAAGGAGAGTTGCATTTCCAAC
CTGAAACTAGGGAAATTTGATAGAGCTCAGGAAGAAATGTGGCAGAAATA
GAAACTGTTTGTAGTTTAGAGGATTACTGCTAAGAAACTGATTTCAGAAC
CAAAAATCAAAACTTCTTACAATTTTATTAAAAGCAAATCAACATTTTAT
GAAACACCTCATTGTTCTAACATAGGGGACCAACATTTTAAGTTCTGTAT
TAGTGTGTTTTTAACATCAAAGTTCGATCTCTAGAAAGGCTCATACACAA
GTTCCTTCCAATTGTAACTCACTTGATCACACAGAAAATTCCTTTTATAA
ATTGTCTTTTCACAACATTATCATGATTCACTTAGATCATTAACAACATG
CTTGGACTTTCTGGTTTGTCCCATACTTCCTCTTTCATGAATAATCAGTC
ATTTAATCTAGAATGGAAATTTACCACCCAAGATTTTTTCTCACATAAAT
TATTTTCTTTTCTTTTCTTTTCTTTTTCTTTTTCTTTTTTCTTTTCTTGT
CTTTTCTCTTTCTTACCAAATATACATCTTACCCTCCTTATACGCTTGCA
CACAGAATAGTCTTTCTCATCAATAGTGGTTTAATCACATACATTAATTA
CAATGTTAACTCTTGGTGACCCTAATTTTCGGTAAAAACCTAGAAAGCAA
GCAGCTTTTAACTATCAATCATGTACCAACAATCCAAGAATATACATTTT
ACAATTTCCAGGAACACATGCTTTCTAACAGAACCGACTCTTTAATATGG
AACAGGATATTTTTACTAGAAGATCCACTTACGTTTCTCAGAAATAAGAA
GCCAAAAGTATATGAACCTAAACCCATAATATTCAGCAGTAAATGTCTCA
GCATTACATCTTATCTGAAAATGATCTAGACATTCAATGTATATCCATCA
TTTAATTTAGCTTAGCAAACCCTAAGGATATCATTACTAAAGACATTTGA
GAAATCTTCTTAAGTAAACATATAAAACATAACCATTATCCACAATTCAT
TTATGAACTTTTATTTCATTTATATCCATCCAATTCATTCGCTTTTAAAA
ATTATATTTGGAAAACTTCATGACACACTAGACAAATCTAGCTACCATCT
TAAGCTAAATTTTTTCATCAGCAAAATTTACATTATTGCATGCCAGGCAA
GTATCATAAAAGCAAGAACCCTAAAGTTAAATACATGCATATTTTTCTAA
TAACTCAGAAGACATAGTGATTCCCACCAAACCAACAATATTAGACTAGT
CCCACCCACCAAAAGACTACTAAGTTACATGAACTTGAAAAGCATTTGGG
CTTATTTGCTTAATTTACGAGTACCCATTCATTTATAAACCAATTTGGTG
GCATAGACAATATACAAACAGATATGTTTACACATATATACACAAAAATA
CAAAAAGGCGCAAAGATTTTACAGCTTAATTTTAAAACTCACTAGTTTAA
AAGGACAGTTGGATTCAAAACTGTGTTTCTGTAAATGTAATAGGCTGAAA
TTTATCTGTCCCCCGTGGCCAAAGCCTTTACCGAATTTCAGATAAAACAG
CGTATAAATTTGTATCTCAAGCACAGAGAGAATTTAAGTTTTCTCAAGAA
GGAGTTTGGGTGTGCTAGAGGAGGATTAAAATGCATGCCAAGGTGACATA
AATCATAGGAATTTATCACACAATGTTACAAAGAAACATGCAGATGAGCC
TAGAGAAAATGTAGGAACCTTTTCAAGATAACCAGCTGAAAGAGTGAAAG
AGGAAGGAGGGAGTGAACAATAGACCAGAGACGGTGAAAAAGGTGCAGAA
GTCAGGCAGAGGTGGGCAAGGGGGCTAGTGGAATAAGGGTCTTTCAAAGG
TGTTTTAACAGTTTTAATTTCAATCTTGCAAGCACTATCTCTTTCTGTTT
CATTAAGGCACTCATTCAGTTGGGAATTATTTCTCTAAGGGAGGCAATGG
TAGCCTCTTGATTCAATTTAGAACTTTCGAAGGGCCAACTGAGAATTAGG
CTTCCTTTCCATTTTCTCTGGGAGGCTCAGTATTTTCTTTGTTCCAATTG
TGCACGCAAATAGGCAAGTTTGGAGTTTCAAAGGAGCCCCCATTTTTGCC
AACAAAGTTTAAGATTACCCTAAGTAGTTTTTGTCCAAAGACTTAGCCTA
TTACAATTCCATGGACCATAATTTTTGCTCATAAGGCCAGCAGGAGAAAA
GCAGTGCCGGAAGGTGGCTGATCAGCAGGGGAATCTTAAAATGTGGGTTA
TCCACTGCACTGGCCTGATGAAAATGTCCCTTCCAATGCCCTTAGTGTGA
CCCTCCCCACGAAACCTTACAGACGAGCCAAGTTCGACTGGGAAGAATCC
CCCCAAAGGCAGAGTCAACTGGGATGGCAGATCCACCATCTCCCCAAGGT
GCTGGGTAGTCCCACCTCCATCCTGAGTACCTTCTTAGCCACCAGGGCCT
GATCCCAGCAGGGACAGTAGCTCCTTGAGACCCAGCAGGAAGACAAGGTC
TGTATTCAGAAGTCCCAGGTGCCAGGTCACAATGACCTCTCACAGTGGGG
GTGCCAGGTCACACTAACCTTTCACAGGGACCTGGGAGCCCTTAATAACC
AAGGCTTAGTTCCTGTTCTTTGAAACATAGATTACAACAACCGGACCCAC
TTCCCATGGTAGCTGCAGGGGTTAAGTGAGATGCTGGCTCTGAGGCTGCT
GTGTGAATTGCCCGGTGTTGTTGAAAACTTGGTTTATTGGATTCAATAGA
ACTCTTCAGATGCCAGCAACAGGAAATCCAGTTCAACAAAACTTAAACTA
TAAAAAGAATTTATTGGCTCACAGAACTGCAAAGACCAGATGCAATGCCC
ATTGAGACTTAGAGCCTGAAGGATGTGGCCATGGGCCCCGATGACCTCTG
GCCCACACTCCTGTTCCTTCGGTGGCCACATCCTGGGGCAGATTCTCCCC
ACTGCTCCCTAGACCTTGACACTGAGGGAGGAGGCCCTCTGCCCTGACCC
TGGCCCTAGGAGAGAGCACACTGTCCTGGTCGCTCCAGCAGGGGGCCCAA
GTGGTCAGGCCTAGGCCCCATGCCCTCCTGCCCCAGTGAGTGTCAGCTTA
CCTAGGCCTCAGAACCTGAGAGTAGGGAGGGCTGGGACCCTGAAGGAAGG
TCAGGGAACTTTTACGAGAAGAGGGATAGACTTGGAGAGGAGTAAACAGC
AGAGGTCTACCTTGCTGACCATCATTCCTACTACTGTCGCTGGAGAGAGG
TTCCTAGAGCCGGTGAGGCCAGGAGCCCCTGCGTGCATTTAGTCTCCACA
CCCTGGTGTGAGAGAGCCATAGGCTTACTGTCCTCAGGTCAGAGTCCTCC
CTGGTCACTCTCCTCGGCCTCCTAGCCTGCTGCCTGCACAGGGAAGCAAT
GAGCATTCTCTTCCTGAGTGCCCTGCAATGGAGCCACACCTCCAGGCACT
CCCTCCCTGTGACCATGAACAGGGAGGAGGCCTGACTTCTAGTGACCTCA
CCAGGTGACTCAGAGCACCATATTGGGCCCAGGGTGTGTTGCAGACCAGG
GCCCAGGTAAAGGGTCTCATTGAGCTTTTATTGGCCCTGATGTTTACAGC
AGAGAAGGGAGAACTGACGCTGCGTGGGAGAGACAGGCTGGCCTAGGGCA
GGGGCTCAAGCACAGGAAGAGGCTGTGGTCCAGGACCCAGGAGCCGCTGC
CCACCCCCAGTGCTCTTGGCACTTTCTTTGTTCAATTGGGAAATAAGGTA
TGCACTACATGCATCTCCCATCAGCCCCTCGCGCAGGAGTGGTGGGGAGA
GGCAAGCTCTTCTGACAGATCAGCCCTGCTGTGTCTCCGGTGGTGGCATC
CTGCTATCTGCACTTGTGTTTGTACTTGGTCCCTGCCCTGAAGGCTCACA
ATCTACCTGATATGGGGTCAGTCCAAACAGTGCAGAGAGGGCGTGTGCAT
GGCAATGCAGGGCACCCGCTCCCTTTCCCAGGTGAGAGAAAAACAGGGCT
CAATATGTATTCCTTGGGCTCTGTGGAGGTGCAGGAAGGCAGGAAATCGG
GAGCCTGGGGCCTTTCAATAAATGAGGAAGCCACAGACCTGGGCAAATAA
AGAGAGGGGAGAGAAAGGGCCCTAGAAGCAAGACTGAGGAAAAAAGAGAT
CCCTCCCAGAAGTGAGGTCAGGGCAGGTGGGAGCATGGGCCCAGACCAGG
GGCTGGACCCAGAGCAGGGTACACGAGCCCCAGAGTGATCATACAAGTGA
TACAGGCATTGTGGGGAATCCAGAAAACAAATAAGGATGTGCAGGGAACC
TCTTCATTAGAGCTTCCACGGGCTGACCCTGCTATGAGCAAGGTGAGGAC
ACAAGGAGAGCACTTCTCACCAGGAAATGCCAAGACAGCATTAAGGAAGC
CTGCCTGCCAAGTGTGTGTGTGTGTATGCGTGTGCATGTGCATGCGTATG
CCTGTGTGCGTGTGCGCCTGTGTGTGCGCGTGTGCCTGTGTGTATTGCCT
GTGTGTATGTGCATGTGTACCTGTGTGTGTGCCTGTGTATGTGCGTGCAT
GTGCCTGTGTGTGTGTGCTCACGCCTGTGTGTGCATGTGTGTGTGCATGT
GCATGTTTGTGCCTGTGTGTATGTGTGCATGTGTGTGCCTATGTGTGCCT
GTGTGTGTGCGTGTGTGTGTGTGTGTGTGTGTGTGTGTGTGTGTGTGTGT
GTCCTGCTGCAGCCTCTTGACAGCTCCCAGGGTTTAGGCTCCAAAGGCAG
AGGGAGGGAGTTCCAGGGCTGGCACTGGGACTCCCAGCTTGTCTCAGATG
TGCATAGCAGGAAAGTTTCAGGGAGGAGATGGGAGAGCAGGGTCCTGAGC
CTCTTCCCATGAGCTGTATATACTATACTAACTGTAGAGCCACCCAGGCC
CCAGAGAAAGCAGGGCCTCCTGTAGAGAAAAGTGAGGAACAACTGGGGAG
GGAACAGGGGACCATGAGGGAAGCACTGCTAAATTCAGAAGAAAAATATG
ATAGTAATAATGGTGGCCATTATTGAGGGCTGTGAGACCAATTCACTCCT
CACAAACACCTCTGTGAATTAAGCACTGCACAGAGAAGAAGTTAGGTGAC
TTGCCTGAGGTCACATAGCTAATAAGCCGACAGTGCTGGGATAGACCAAG
AGAACAGCCTGGCTTTGGACCTAAAATCCAACACTTCGGTTATGCCGGTG
AGGAATCCAGTTCTTGCTGGGAAGGCACCCACATAAGGTCTCTGGCCTAT
AACCCTGGGTGCTGAGTTCCTTCTCCAGAACTGGGGGTGGGGGGGGTGCT
GGGTCAAGGCAGCCACACTCCACTGCATCCTTGGGTGAGCCCCAGGTTGG
CCAGGGTGGTGAAGGCGACGAGGCTCTCAGAGCTCATGAGGTTTGTCTGG
ACTGCTGGAGACAAGGAAGCCCCGGGGAGTGCCCAAGGTCTGTGGGGAGG
GAGTGGGGTGGAGCTCTACAGCTGGTATTGTCATGCCATTCTCCTTGGAG
TGCTCCCCTTGCTCTATACAGGCCAGGAGAGGTGTCACTGTAAGGAAGAA
GGGGCCTAAGGGATGGAAATGCAGCATGAGGAAATAGTTTCAATGCCATA
GAGAAAAATATCTAGAAGTGTTCACACCCATGGGGCTTAGTAATTATACC
ACTGTCAGTCAGTCCTAAGGCAAAGAACTATTTGCAAAATGTTATCCATC
AAGTCACTATTTGTGAAGGATGAAAGTTAGAGGCAGCCTTAACTTTCAAC
ACCAGCAGAGGGTTTAAATCTTTTAATAATTCCTGGTTGCCCATGGCTGC
CCATGTAGCCATTCAAGAGATGGTGAAGAGCTCCTCTCAACACAGAACAA
TGTTTACTGTAAAAGGTAGGATACTAATGGCATACCATGATTTTTCTTGG
TAAAAACATGTTTATGGGAAAAACTGGAAAAAGTAGAAAATTCAGTAAAA
TTTAAGTAGTGGTATGGAATGATAGGTGAATGAAACAAATATTCTATCTC
CCCCAGTAGAGTCAAAACACAGTTATTTTTGAAAGAGTATAATATAAAAA
TTAGCAGGCAGTTTAAAAACAAGCATCAAGGCAGAGAGACAAAGTCTGGT
TCTCTATTTGTTCGGCAACATCAAGATGGCAGGATCCTCTGGGTCACCAG
CCCGGCACAGGACTGAGGACATTATAGGTGCTCACTGAACATGTGTTGCA
CTATACATGCAGGTTCCAGAAGCAGAGATGGAATCCCTGCTCCCCAAGAG
TGGCTGGTTATCACACAGCAGTGCTGATGAGTAGGCACACGGGCTGTGAT
AAACACCCCTTGGGGGAGGGTATGACCTGAATCTAGTTGGTTGGTCTGCA
GACGGGGCCAGGCTGGGGCTCCCTCCCCCAGGGAGGAGGCTGCTGAGGGA
CCAGACAGGGAAGGGGTGCACGGTGAGGCCTATTGGGGTTTCTCATGTTC
CCCCAAAAGCACAGAATGTCTTCTACTGGGTGAGACCAGGAAGTGGAACA
GACAGCCAGACCAGGCTTTGAGCGAGTCATTCATGGAAGACAATGGAAAC
CCCATCGTCGTGTGCCAGGCGCTGTGCACAGAGGGTAGACCCCAGGGAAA
ATGCAGAGCAGCTGGGGTCCCAGGAGGACACTCTGGTGCTGTGGTGCCCC
TCCCTTGCCACACTGCATTTTCTCAGGTGGGGGCAGGAGGGCAAAGTCTG
CCTCATGTCCTGAGCGGGAAGAGGGGGTGGGAGGTAGAATGGCACGCAGT
ATGTGCCGTGGAAAGCCATGCTGGAGGAATGGAATGTGCAGTCAGCAAGA
GCAGAAAAGAGGTTCTCGGGGCTGGATCTAGTGAGGGCCCACCAGTGACC
CCAGTCTGTGCTCAGGCCTTCATTTAGGGAGAACTCACCAGGCTGGGTGT
GGCCTCGATTCCTGAGATGGCATCCCTGGGAAGAAAAAGGGAGGGCTTTC
CAAGGGGGCAGGGATGGGCAAGGCACTAAAGTAGGCTTCTGACCCTGAAG
GGTCATGCTGTGGGTGGGCGGTGGACTTGGTCAGCCCACATGTTGTGGCT
CAGTCCCTTTGAGCAAGAGTTTCCCAGCAGCTTGGATTCTGTACCTGGGC
TGCCTGGGCTTTAATCCTGGCTCTGTCACTTACTACACATGTGGCCTGAC
CAAGTTATTTTACCTCTCTATGCCTCAGTTTCTAATCTTAATCTTCTCAG
TCCTACTAGTTCTTAGGGCCCCGCTTGACAATCTCCTCCTCCAGGAAGTC
TTCCTGCTGCCCAAGGTGAATTAGGGCCCCTCTTCCATGCTCCCATAGCA
GCTATGCGCAACAGCTTTCTTCCTACATCACAGCCACGTTTTCTCTTTTT
GTTTTCCATTCTGAGACCGTAGAGTTTTTGAGGTCAGGAGATGTTCTTGT
GGCTAATTGTTTTGTTTTTTAGGTTTTTTTTTTTTTTTTTTGTATTTTCT
CAGTGCTAGGAAGTATTTGCTGAAATTCACGTAAAATGGAACTAATGAAA
ATAACCTTCCTCATTTGGTGGTTTTGAGGATTAAAAGAATCAACCTTGGC
AGGGCGCGGTGACTCACGCCCGCAATCCCAGTGCTTTGAGAGGTCGAGGA
GGGTGGAACCCTTGAGGTCAGGAGTTTGAGACAAGCCTGGCCAACATGGT
GAAACCCCATCTCTACTAAAAATACAAAAAAATTAGCCGGGTGTGGTGGC
AGGCGCCTGTAATCCCAGCTACTTGGGAGGCTGAGGCAGGAGAATCTTTT
GAACCTAGGAGGTGGAGGTTCCAGGGAGCCGATATGGCACCACTACACTC
CAGCCTGGGCAACAGAGCGAGACTCCATCTCAAAAACAAAACAAAACAAA
ACAAAAAAGAATCAACCTTGTATTAGAACCTGGCACAGACTGTTGGCACT
GTTAAAATATAAAATAGTGGTCAGGGTGACTTGCAGAGGAACAGGCTGCC
CCACCAGTGACAGTGACAAAGCAGTGGAGCACTTGCTGCAAACATGGCCT
CTGACTCAGGAGAGGCAGTCACTGTTGAGAGTTAATCCATGATCACACTT
GGGTATCTAAATTGCCAAGGAAGCTTCATCAGCTGCTGCAGGAGTGGGCA
GTGTGGCCCACCAAGTTCATGGCCAGCCAGTGGTGAGAAGAGACAAGGCT
GAGACCCAGAGACAACCCAGAGGCCTTCAGGGGGCAGAAGGAGCGGGGTT
TGGGCAGAGAGAACAATGGCATGGAGGTCTAGGAGCAATGAGGCCTTTTC
CAGTCACGAGGGCCCTTTTAGAATTTATTTCTGCTTAATAGCACTTAATA
AACATTTTTGAGCAATGGGATATGTTAGGTCAAATGGGGGATATAAAGAG
AGTTGACACAGAACCTTTGCCCTTGAAGAGCCCACAGCTGAGTTGAAGGA
AGAGACCTAGAGCACAGTTTACTAACATCAAACCACCAACCCCCACAGCA
AGAGCAGCTTCCCAGCATTGAGACCTCACTATGCTCCAGGCATTCCATGC
ATCATCTCATATGCACTTCATAACAGCTCATCACGAAGAGAAGTCACCGC
AGATCCAATGGGTTAAGCAAGCTATCGAAGGTAGTCAGCAATTTTGTCTG
ACTACAAGTTCGTGTAATAATAATGTGAACCACTCCTTGAAGAGTAGAAT
CCAAGCATTGTGAGGCTGGCAGTGCAGGCAGGGGAATAGCATGAGCAAAA
ACCAGAGGTGGGAATGAATTTAACTGCCCCAGGAGGAGGCCCAAGACAGC
TGGAGACAATTCAGGCAGACAGGAGAGCCCGTAGCTGCTCAGCTGCTCCC
TGTCTTTTTCCTTCCCTCCGCAACCTTGAGAAAGTTGTCGATGGGTTCTA
TTTGCATTTTCTCCTATCCAGTTCCTCCTGCCCACATCACCCACTGCAGA
CACTCTTGCCAAGTTCCTCAATGCCAGCGTTGCCAGCCTGGTGGCCACTC
CTCAGGCCTTCTCTTGCCTCCCCAGCAGCCTGGCCATGTGCCCGCCCTCC
TCCATTTTGAAAGAGTACCCTCAGCTCTATCAACACCTCCTCCCCTGGTT
TACTTTCCTCCACTCTGGCTCCGTCCCTTGTGCCACCTTGATATTGCTGT
GGCCCATCCTGGACCTACTTCTCATCAACACAGTCTCCCTAGGAGACTCA
TCCCTTCTTGAGGCTTAACCACCACCTCCACGTGGGATTTCCAAGCCCAT
ATCTCTAGCCCAGTGTCCACCCTGAGTGCTGGACCCATATACCCATCTGA
CTCCTGAAGATCTTTACTTGGGTATAGTCTGGGAAATTTGAATTTCAAAT
GTGACCAGGATACTCACTGGCTTCCTTCAATAGTGACTGCTCCTTCTGGG
CCCCCTCTGTCAGGAGTGGGCATCACCAACGCTAGTTGCCCAAGCCAGAT
CCTGGGAGTCATCACTAACATCTCTCCTCTCACTGATATGTGGGTGCCAC
CTCGCTTTGACATTTCTTAAGCTTTTCTCTCCTTCCTACCACCATCCCAC
TGCTGCCATGCCAGCTTTTATCATAGCTCCTGGGATTCCTTCCACAGACT
ACCTAATTCCACTCTGGCTGCCAGAGAGGTCCATGTGAAATGCAAATTAG
CCCAGGTCACCCTTGGTTAGAAGCCTTTAACGGCTCCCCATTGTGCTGGG
GAGAAAACCTAAACTCCCAGCCTCTTTGCTTCACACACACATACCCACTG
CGCTTGGTCTTGAGCTCCAGCCAGACTGAAACCATGGAGTAGGTTTACCC
TCTTTACCCTAATCCTCTCAATCCTACTAGTTCTTAGGGCTCAGCTTGAC
AGTCGCCTCCTCCTAGAAGTCTTCCTGCTGTCCAAGGTGCATTAGGGCTC
CACTTCTATGCTCCCATAGCAGCTGTGCACAATAGCTTTCTTCCTACATC
ACAGCCACACTTTCTCTTCTTGTTTCCCACCCTGAGACCATAGAGTCTTT
GAGGTCAGGAGATGTTCTTGTGGTTTAGCTTTTTTTAAAATTTATTTATT
TATTTATTTATTTATTTATTTATTTATTTATTTATTTGAGACAGAGTCTT
GCTCTGTAGCCCAGGCTGGAGTGCACTGGCACGACCTTGGCTCACTGCAG
CCTCTGCCTCCCAGGTTCAAGCAATTCTCCTGCTTCAGCCTCCCAAGCAG
CTGGGACTACAGACACCTGCCACCATGCCTGGCTAATTTTGTATTTTTAG
TAGAGAAGGGGTTTCACCATGTTGGCCGGGATGGTCTCAATCTCCTGACC
TCAAGTGATCCGCCCGCCTCAGCCTCCTCAAGTGCTGGGATTAGAGGCAT
GAGCCACCGTGCCCAGCCTATTTTTTGTATTTTTTCAGTGCTAGGAAATA
TTTACTGAAATTTAAGGATGGAAAGTCTGGGAGACCGACTCTAGGAGTGC
TCTTGAAAGGAAATGGGTTACTCCATGTGTAGGTGTTGAAAATGTAAAAC
ACAATATTATTTCTCCCTTTTTCCCTAATAAAACCCCAATTTTGTTCAGA
GGACAGCAATAGTGTTAATTACACTTCTTAGCATCCTTTGCAGGGGGTAG
CTATGTGACCCAGGTTTGGCCAAGAAGACTTAAGTAAAAGCAACTGTGCA
CGCAAGGGAGAGATTTGCTCCCTTGATGTAAGCATGGCCCCTTCCTGTTT
CCTGTCTTCCACATCTTTCCTGGAATTAGGAGAGGAGGCTGGAGATGGAG
CAGCCATCTTGTGACAATGAGCCAGCCACAGGGATGGGAACCACAGGCTA
AGGAATCTTGAAGGGCCTAGGTCATGACCCCTGGACTACCTACTAGCAGA
CTTCTAATTATTGAGAGACAAATAAATCTCATTTAAGTCATTTTGGTTGG
CTCTCCATTACATTTAGCTGAACTCCACCCAGTAAAGGTCAATGAAGGAA
GATAAGCATCTGTGAGCATTTCTGAATAGGCTTTTCCTTTGGAGATGATG
CTGGCGCTGACGGGAGGCAGAGGGCCAGGTTAATGGAACAGGACTAGGTG
AGGGCTGGAGAGGCTGATCCTCCCAGGGGTTCTACCAGAGGATCACAGTG
AGGCCTCAGGGTTTGAGAAAGAAGCTCCAGGCATGGTAGCCCTAAGGTGT
CTGTGACCTTGGCCTGGGGGTTTTCTGGTCCCTGTTTGGTTCAACTTGCA
AAATGGGGCTGTTGGACTAGACAACTTATCAGAAGGCATAGTGCTTCAAG
TCTGGGCTTAGGAGTCAGACAAGGCTGAATTCAGGGTCCAGCTCCACCAC
TTGCTAGCTGTGTGGCTTTCAGACAGCTTTCTTAGCCGACTGCTCTAGCA
TCCTTGTTTGTGGAGTCCAGACTCCATCAGTACCTATACCTGTAGGGCTA
ACGTTGGGGCTTCATGAAATCATGCATGTGGGTGCTCAGCAGAGGGCCTG
GTATGCCCAGCACACACCAAGTGCTCCATGCTGGTACCTGTCCTTGTGTG
CCATGTTCTCCCAGTGGACAGATGGGACCTGGAATTCATGCCTCCTCTGG
GGCTGGGGCTGGGGCTGCTACAGTGCTGTGGAAAGCCTGTCAGGATGTAC
CTCGCTCTGTGGCTGAGTGAAGAGGCCAGAGGCTGCCCACTGTTCAACAT
CTTTGGAATGCTGCCGTGTCCAGGGAACTTGGGTCGCAGGTGGATAGAAG
CCCCCAAGCCCAGGCCAGGCCAGACCAGGCCCAGAAAGGTATCAGGTCTT
GGGTAGAATAAGAAGCCTATGTGGTCTATCTATCCTGTGGCCTTTGTCTT
AGCCAAAGCCTCCTGGATATTGACGGCATCTTTTCCTGGGTTTGGGGTGG
GCTTGGAGAGGTTCCCTCTGCACGGGACTACAGAAGTGAGGGAGCTGGAA
AAATGCCTCCAGAGCTGGATTCCTCACTGCAGCTGCTTTTGGAAGAACCT
TCTTATCCTTCCCTTGAGAAGCCTCACTGTCTCTTCTCGGCGCTTGGAAT
CCTGTTAGTAGAAAATCTTGGGGAAAAAATAAACCCACTATTCATTCCAG
CACTGAGAAGGAGGTCAGTGGGGTGCGTCCTGTTTGTCCACACCCCCCCC
ACCCCCTCTACCCCTCCCCCCAGCACCCCAGGCCAGACCCAGCCATGCTG
ACACTTGACAGCCCCTGCAGTTTACCTTGGCTCAGGTCCAGAGACTTCGG
GCCCAGCCCCCACCTTTTCACCTTGTTACCCAGCCCCCTCTCCAGCGCAA
AGAGCAGTTTAAATATAAATCAGTCCTCAACACAAACAGAGCTGCAGCGA
GTCTCAGAGCCATCACCGGAACTGAGGGCCCAGGTTTTCTGCGAGTGCAA
ACAAGCAGAGACTGGCTCCGTAAATCTCCCCAAGAACGTCGGGAGGGGTG
CTGGGGCTCCCCTCCACCCCCGGCTCACGTTGGCCCCTCCAGCGCCCGCC
TCGGGCACCCTCATATCCCAGCCTTCCTGCCCCCCAGCCCTGCCAACCTC
TTTCTCCATCCCCTCCACTCACCCACCTTCGCTTCGCCCACCCTCTCTTC
GCCAGTCCCCAGGAACCTCCACCCCCTTGCAACCTCCTGCCCCCGCACAG
CCCTCACCTCCCCTCCCCGGGCCCGCCACCTCCTTCCCTCCCTCTCCGCC
TCCCGGCAGGCGGGATCTTCTCGGGGCAGTCAAGCCTCGCGGGTCGCTGC
GGCGTCACACCTGTCTGAGGGGGCGGCGGCGGCGGCGGCGGCGGGGCGGG
GGCTCGTAGCGCCTTTGACACCCGAGGAAAAGAGGGAGGAGGGAGAGCGC
GTTTCATCATCGGCGGCGGCCACTTATAAAAACTTCTAGGCGCGCACTCG
CTGGCTCAGTCTCCGCTCCGCGAATCTCCTCCGGCCACTGCCGCCGCGGT
CGCCTCTCACCCGCCCGGCCGCTCCAGCCCGAGGCGCCCCGACCCCGCGC
CACTCCGCGCCCGGCCAGCCGCCCGCAGCCATGGGGCTCCTGCCCAAGCT
CGGCGCGTCCCAGGGCAGCGACACCTCTACTAGCCGAGCCGGCCGCTGTG
CCCGCTCGGTCTTCGGCAACATTAAGGTGAGCGGAGTCCGCGGGGTCTTC
CGGGCCGGAGCCGCGCCTGGCGCACCTTCGGCTGGGCACCCGCTTCTGTC
TGCCGGAGCCAGGAGGCTCCGGGAGCCCCGGGCTCCCTCCTGTGCCGCGC
TCCGATCTCATCCCCGGGCGACGCGTGCTGGGCGTGTGTGTGCGAGTGTG
TGTGTGAGCGTGTGTGTGTGTGAGCGTGTGTGTGTGCGTGCGAGTGTGTG
TGTGTGTGTTCACACGCCTTCATCGGACTAAGTAATGGGACAGTGGAAGT
ACCTGCTTACCGCTTACTCGGACAACTCCAGGCAGAGTTATAAGCGAGAG
AATTGTCCTCGATGATTGAAAAAGATCATCTCGAGGAACGCTTTTTCGGC
CTCCAGCGGGCGGTTACTTCGTCAATGGGATGGGCTTGGAGGTGCCAGGG
GGACCCCCCGCGCTTTGGGGGACAGACTGTGTCTCGTCCGAGCCCCCTCG
TCCTGGGGTGGGTGGCCTAAGGGATGTTCTCCTTCCCCAGGCTTTCTTCT
CCCGCCAACGGGGGCGGGGGGCTCTGCTACCTGGCTGTGCCGCTCGCAGT
TATGTTTAGAATTCCCTCTCAGCGGGCGGCTTCCCCAGGCTGGCAACCAA
CCCGCGTGCTCAGATTGGCTTCCCTGGTCTGGAAATGGCCCCAGGAAGGT
CACAGCTCTCACGGTTCAGTAAGGTCTAAGCAGGTCCACTCACCAGGCAA
GTTATGGGGAGCGGGTGAGAAGATAGGGTGTTGGCAGCTGTTAGGATGAG
AGCACCTTTCCCATGGGGTCGGAAATGCTTTCCACTCCCTCACAGGTCCA
GGAGGACCTGGAATGGTTCAATCCGCACTCGGGCTCTTTTGCCCGTTCTG
CCTAGGCAGAAGTAACAATGGAAGATACCTCCTGAGAGGCCAAAGCTGCT
GCGGTGAGGATGTGGGAGACTGCAGAGACTGCATCAAGGCCCCTTCTGGT
CAGGGTGGGATCATGGTGTAGAGAGGTTGTTTCCCACAAACATACAGTCA
GATGGGACTGAGTAAAGCCTCCGGTGGTGCAATTAAGGGGAAGCCAGATT
ACACAAAGGGATGATGGGGGTGTCCGTGGACTGGTTATATGTGTCCAACA
GAAAGGGATCTGGAGGTCAGAGTGGATCTCACCTGCATACTTACTCAGCT
GGGATCTCCACTCCTGCTTCTGCTCTTTCCGCACCGCAATAAATACAGGC
TGAATTGATTTTGTAGTTGTATATGGATAAGCAACAGAAATTTTGAGATA
GCTTAAAATAATCTCCAAATCTCCTCATTTTTGGCCAGACCTGGATGGAA
ATGTTGTGCGCAGTTTTGGTTCCACCATTTTGGAGAAATTTCATCAGGAA
TTTTGGAACCTTTGGTATTTATAAATTAGGAATTGAGATGAAAGGGTAGG
GACTAGGATTAGATGGTGTAAAGAAAGGGGCCTCTGCAGGAACCAGGGTG
GGGCAACCTCACTTCATTTTCACCAAGAAGGGCAGGAGGGACGGGTGTCC
AGAACAGATTCCTGACAGTGAGGGTACAGCTGAATGAGACTGATCCGTTT
AGACCCACCCTGGTTCCCTAAAATAAATGTTTATGGCAGCAATTTATTTG
GAGATCTATATAAATAAGATGATTTCTCATGTGTTGATGATTTAACCACA
CGTACCTGAGGGCTGGGCTCAGGCCAATTGGCCTCCTAAAGGAGTTAAGT
ACTCATACTGGCTAAGAGTTTTTCTTGACTCTAAAAATGAGATTCTGTTA
GTCATCACCTCCTTTGATCCATACTAATTGCAAGACCAGAGTTTGGAGGA
CTTCCATTTAACTCCACTTATCAGCCTGAAACCAGGTACGAGGTTGGATA
GCTTAAGGTAAATCTTGAATCTCAAATTCCATCAGTTAAAGTCACTTGCA
CCTTTCCCTCCTGGGGCAATAACTGGGAGCTCTGCTATTTGAAGGGATAT
TTCCTCCCAATGAGAGTGCTTGTAATAGCCCTGCCACTTTGCAGTGGGGG
ATGGGTTGCACATATCTTGGGACATGTATGACATGGTGGTGTAGCAGGAC
GAGCCACAGACAAAACTCCTCAGACACTGAGTTAAAGAAGGAAGGGGTTT
ATTCAGCCGGGGGCATCAGCAAGACTGCTGTCTCAAGAGCTGAGCTCCCC
GAGTGAGCAATTCCTGTCCTTTTTAAGGGCTCACAACTCTAAGGGGGTGC
ACCTGAGAGGGTCGTGATCGATTGAGCAAACAGTGGGTACGTGACTGGGG
GCTGCATGCACCAGTAATTAGATCGGAACAAAACAGAATAGGGATTTTCA
CAGTGCTTTTCTGTACAATGTCTGTAATCTATAGATAACATAACCGATTA
GGTCAGGGGTCGATCTTTAACTACCAGCGCTAGGGTGCGGCTCCGGGCTG
TCTGCTTGTGGATTTCATTTCTGCCTTTTAGTTTTTACTTTTTCTTTCTT
TGGAGGCAGAAATTGGGCATAAGACAATATGAGGGGTGGTCTCCTCCCTT
ATTCCCCTCCTTTGAGATTCTCACTCAATAGTGGGAGTTCTCACTTTCAT
TTTTACTACCTGTGTCTTCTTGCAAGACAGATCGATAGTGATTCATATAG
TACACTTGTGCTGAACCTTTTTGGTGAACTAAGGTAGCGATGAAGCTTTT
TATCATTTGAAGAAGTATAGGTAGCAAACAAGGGAGCAGTAAGCAGGTTC
CTATTACTATTATAACTCTTATTATTAGAGTTTTAAATTCTCTTAGCGCT
GGGAACCATTTTCTAAACATGGCCCCAGGATCAAATCCATGCCACACTTG
CACAGGCACATGTGCCAGTTTTGTCATATCTCTTAACTGTGTCTTCAACT
ACTTGCCCTTGATTATCTATATGTAGGCAGCAATTAGAAAGGTTAAATTT
CCTACAGACCTCTCCTTCAGCTGCTAGCAAGTAGTCAAGAGCTAATCTAT
TTTGATAGGTAGCATTTCTCATCTGAGTTTCTTGCTGGGCCAGAATAGTC
AAGGCTCTGCCGGTTTTATTAGTGATTATTTTTAAGACAGCTTGTAACCG
TATGATTCAGTTGATCACGTAAATGGGGGTCTGGTATCCTCACAAGCCGT
CTTGTGCCTAGCAGGCCTATAATATTGTATGATTTTCTCAGGGGGCCATT
TATCATTTTTTCAATTTCCTATAGCTATGCTTCTCTTTTCGCGGGAAGCA
TAGACAGGGAAGCCCAGGAGTTCGCCTGTTTTTATGGGCAGTAGGAAAAA
GATGGTTTAATAGTGCCAATAACAAAACTACCTGCCTACTGGTCAGGTAA
TTTGGCGTAAGCTCTATGCCTACGTATCTAGTATAATCCAGTGGGGGCTG
TCTAGTCCCGGTGGGACTCCGGGTGGGTCCACACGGTTTGCAACTTTGGG
AATTTACTAAATGGATTTCTCTCTGTGTGATTAGAACTCCACCAAATGAC
TGTTTTTGTGATACCATTATAAGTTTCTGTCCTAGACAAAGAAGTCGTCC
TATGGGGTGAGTAAATTCTTTTCCTTCTCTAGCTATGCAATATTGTCCAA
TAATTGAGGCTTTTAGGACCTAGAAATTATCAGGGTGATTCTTCTGAGCT
GGGAATTCATCAGGAACTGGGTCTGTAGGTACTAATTCTCGGGCTTCCCA
TGGTCATTGATCTCTTATTACAGTTCCTCCACATACATAACATGAAGTGA
CATTGAGAGAACATGCTCAGCTAATTGCAAAAACAAATTTCTTGTTTTTC
CTGGAATTTCTGGTACTGGCACATTTAGTTCATCATAGAAAGTTTGAAAC
ACTGGCTTAGGAGAGCGTTTGTAAACTTCTCTTCAAACCAAGATATTTAC
TCGAGGATCCAGTCCAGCCCCATTGATTCCTAAGGTCACACGCTCCTCTT
TTTTCCAGCGAGGATCAAGGGGATTGGTTATTACTAGCTCTAAGGGGTTA
CATTGTCCCTTAGTACAGGAAGGGCCATTTTTTCCTTTCTGAAGGTGGAC
TGGATCTTTTTCATTTTTTTTTTATCGAAGTGGCCTAAATGACACAAGAC
CAGTATTTACATTTCTTTCCACACAGTCTTAATTTATGACAGATGTACTT
ATTTTCTGCTATATAGCCTCTTTTCTAATTAAGAGAACCACAGCTTATTC
CTAACTTATTACTATTAATGACAGCACAGGCATCAAATTTTAAGATGACT
CGTTTGGGCACCCCTTTTTCTTCTGTTTTGGCTAGCACTTTACTCATATC
ATTTGTGAGCCCTCACCAGTCCTCAGTCCTCAATCTAATTTTAAAAACTG
TGGTCATGGGAGGCTCAGATGGGTCATAACACACATCAGGTTGGTCATTT
CCTGGGCTACATACCTTGTATAGAATTATATTATACAACAAGTTCTTTTT
AGAGTTCCAGTACGCTTATAATAACCATAAAATAATAGGACCGTAGCAAC
CTTTTGTCCTACCTCAGTGACTTGATGTATACACTGGGAATAGTCCTCAG
TCTGAGGAAGGTCAGTTGAAGTCCTTATTGTAAAAATGCAAATTTTAAGG
AAAATGAGTCCCGCAATGAGTTTTCTCATGCTTCGGCCGTGCATGGACCA
GTCAGCTTCCGGGTGTGACTAGAGCAGGGCTTGTCGTCTTCTTCAGAGTC
ACTTTGCAGGGGTTGGCAAAGCTGCTCCCGTCCACATACTGCTCACAGTC
TACTGATGTTTAAGGATGGTCTCGGAGGTTGGGCCTGTTAGAATAAACTG
AGTCCAACACCTCTACACAGTTATGTTCAACTGGGCTCTCTGATACTGGG
AGCAAGGTGGTGGGGTTTAGGGTGTTGCAAACTTCAATGGTTATGTGGGG
ATTTTCACATAGCAAGCTTTGGTACTTGGTTAATCTAGCATTTGTTAACT
AATGATGTCCTTTGGTAGTCATTAAAGTTACCACAGCATGGGGAGCGTTT
ATATTCAGGTTTTGCCTAAGGGTTAGTTTATCTGCTTCTTGTGCTAACAG
GACAAGAAGGCCCTTGTTGCTGCTAGGGCCCTTAAACCTGGGGGCCAGGC
TTTGGAAACCCCGTCTAGTTGTTTTGAGAGACAGGCCACTGGCCTTGGCC
AGAGCCCCACAGTCTGGGTTAAAACTCCAACTGCCATTTTTTCTCTTTCT
GACACATAGAGTGTAAAGAGTTTTGCCAGGTCATGTAGCCTCAGGTCTGG
GGCCAACATGAGTTTTTCTTTTAACTCATGAAAAGCTCGTTGCTGTTGGT
TGTAATAGATGTAGTTTATCTAATCTACATTTTTGTTAACTGTCACCCAC
AAAAATATTGACTCAAATCCTGCAGCTATTTGATTTCAAGCTTTAAATTG
ATATGGTATTCGTCATGGGACTCCAATTGTGTCTAAACAGACATGAGAGT
CGAAAGACCTATAAGGGGCTTCTCTCGCTTTACGATGTCTTATTTTTCTC
CTTCTGGTTGATGAAATGCCAGGGTGAAAGGGATAGCCAATTGGACTAAA
GTACAAGTGCCACTCCATTTATTCAGCAGAGTGCCCAGTAAAGGTCCACC
ACAATACCACCACACATCTGCTCGGGGATGAACAAGGGCTGACTGATTGA
TAAGCTCTTGAAAATTCTTAAGCTAACTGCATCCTTTCAGGTCTCCAAGG
AACACTAAGTTTCCTCCCTGTCATGAGAGACATGAAGTGACTTAGTGTTG
GGAAACGGAGGCTGGATGGCCTTTGGGGGCTGACCCGCAGGGTGCCAGAC
TTTGGGATATAGCAGAGAGAGCTTGGCATGACTTATTACTCCAGGCTGTA
GAATCCTGGAAAAGAGCCATGCAGCCTACACCTGGTCAACTGGAGGACCA
CCTTAGTGGAAAGGGGACAGTCTGGGCCTCTGACCTGCCATGTGCACAAG
CATAACAATTGCTTTTGTTTAACGTGCAGATGGAATATTTGATCCATTTT
AACCAAGCATTTGCATCTTGGTATCCTGTCTCAAATTGCTAAAGTTTGTT
TTAAGTCTTTAACTTCCATGATCCTCTAGTAAAATGAATGTATGATTTTA
GGAAATTACAAAAACTGGTTGGGGCAGTCCATCCTTGCTCTTTAGTGGTC
CACAGAACATTGGACCAACTATGGCATGAAAGCTCTACATCGGGGGGCAA
GACTTCTGGTTGGCACTGGGGTCTTTATTGAAATCTCCCCAGATTAAATG
GTCCTACTTTACTAATGCCCAGTCTGAGGAGAGTCAGGAGGGACAGAAGT
ACTTTTCTAAAGCAGAAAGCTGTCTTTGACTTGGCAAGTCCTCACAGGGT
ATAGCAAGGCAAGCATTAAATGCAATAGTTTGAGGAGAAATTGACTTGGT
TATGTTAATAACTAGATGGTCAGCAATAGAATGAGGAAAGAAGAAAGAGT
AATAGAATAGATTAAAAGAGTTAAATTTTTCTTAGCTTTAGTTTGATAGG
GTTTTCCCCTGGGACTATGGCCCACAATTCTGGAGGGGGTGGCGCTTTCT
TGACTCGGGTGTAATGAGTCCATTCTTTTGTTTGTTTGTTTGTTTGTTTG
TTTGTTTGTTTTGCTGTATGAACAGCAGTCTTGTTGGTTAGCAGCACAAG
GTAGGGTCCTTCCCAGGCTGGCTTGAGTTTTTCTTCTTTCCACCCTTTGA
TGAGAACGTGATCTTCAGGCTGGTGCTGGTTTACTGGAAATTCTAGGGGT
GGTACATGTGCTAAAAGACTTAGTTTTTGAGAGAAAGGAAAGTGGAAGAT
AAACCAAGTATATAATTTTTAAGACATCGACCTTTTGTTTTAACTGTGGG
GACCTGGACAGTGGACTTTATAGTCCTTAGTGCCTTTTTACTGAGAAATT
TCCTTTAGCACCTATTTTTATTAGTTTTTAAACCAAAGAAAGCCAAATAC
CATTTTGCATTTAACAGTGCTTCTCATATGATTTTTACACCAGATAAGCT
AAATTTTACCTTTATATTAGTGTGTTATTAATGTTAAACCTAATTTTAAT
AAAACCTTCTAGACATATTTATCTAATTTTTAATGTTTGACCATAAGGTA
AGATTTTATAGACTCTTTTTAACCTTTTATAATTTTTGCTAAAGAGCAGG
TTGGTGCTTTAAGAAAAACCTGTTATGCTTTTACTTTAATGTCCAGTTCA
CAGAAAAACTGGATGATACTTCTTTAACTTTAGCTAATATGTTTACACAC
AGAATTTTCTTTATAATTAACGTTTTAAAACTTGCTTAAACCTTCAAAAC
AATAATTTTTTTTAACTTTTTAATGTAGGTAAAAATGTACATTCTTATGC
CTCCTTATAATCTTTTTACCAAAGGTATATTTTACTTTTCTTATACACCT
TGCATACAAACTGTTTTTTAATAGTTTTAAATTCAGGAGGCCTAGTTACT
TTTAAATTACACCACATTTTTTGCATAAATTCTTTTTTATAACTTTTTTC
TTTCATGACTTTCGCAGATAATTCTTCGACATGCCTCAACTTTTTGACTT
ATTACAAATATTTCTTTCTTTAAACAACTAGTTAATTTATTTCAGGACAA
GAATTTACCATATAATACTCTTTTTACATAAATTCCGCCCCTCCTCTTTT
TTTCTTTTTTTTTTTTTCCTTTAGGATACTTCTGAACTGGTGAGGTGTGC
TCACAATGAGGTTTCCTCTAAAAGTTTTTTTTTTACTTTTTTTTTTTTGT
TAGCAAAGCAGTTGCAGCTACAGATTGAATGCATTTGGGCCATCTGCGGG
TTACTGGGTTAAGGATTTTTTATAGGAAGGCCTCAGTGCTTTCAGGATAC
GCCCTTGTTTACACTGACAACAAAGTGGTATTGGAGTGTGATAGGTTACA
GAGAATACCTTCAATTATCAATTATAGGTTTTAAATTTACCTTGGCTTTT
ATAGGGTACACTTTTTTTTTCTTAACTACTTGTATATCTCTCTCTTTCTT
TCTCTCTTTGACTTTGTCTCTCTCTCTTTGACTTTCCTTTTGCCTCTGTC
TCTTCTTCTCTTTCACTGCCTTTCTCTTTCTCTCTCTCTCTCCTTGACTC
CCTCTTTGTCTGTCTCTTCCTCTCTCTTTGCCTCTTTTCTTCTCTCTTTC
CTTTCTCTCTCTCTGCTGGTCTTTCCTTGCCTCTGCCAGCCACTTATGCT
GCTGTTCTCTCAACCACTGTGTGTTGGGGGCGGGGGGTCTAAAACCAGCT
GTAACCAAGTGTCTATGTACAGGAACTGGTCTGCATTCCCTGGCTTACAG
GTTACCTTGGGCCACACCTTTGAAACAAGGGACCTGTCCAGGCTTCCTTC
TAATGGCCAACTTACCTCTAATGCTGGCCAGTCTATCTTACACAAAGTTT
TAAGTTTTCCTAGTGTCACAGTACTCCATAGTCTCCTTTAAATTCTTTTT
TGAAATTTTTCAACATAGTTCCTAGTAGGGTGGGCTTATTTGTGCCTGAC
CTATGCTTCTTCGAGACAAAACACGCTCATACCACATGCACACCACAAAC
CAAAGAACAGGTAAAAAGGGCACGCACACACTTTTGCAGTTTGCACCAAA
GCAAAATCAAAACCAAAATCAGAGTATCCAGAAATCCAAGCCAGGTCAAA
ACCAAAACCAAAGTATCAAGCAATCCAAGTCAAGTCAAAAACAAAAACCA
AAGTGCCGGTACAGGCATGCCGTGGGTGATGAGGCCACGCTTCCACTCAA
ATGGAGTGGGCAAGTTCCCAAGACCAATCCTGTCAAGCAATTCAAATCAA
GTCAAAACCAAACCAAAGTGCCGATAAAGGCACACCATGGGTGATCAGGC
CAAGCTTCCACTCAAATGGAGTGGGCAAGTTCTCACGACCAGTCCTGTCA
AGCAATTCAAACCAAGTCAAAACCAAAACCAAAGTGCCGATAAAGGCACA
CCATGCGTGATCAGGCCAAGCTTCCACTCAAATGGAGTGGGCAAGTTCTC
AAGACCAGTCCTGTCAAGCAATTCAAACCAAGTCAAAACCAAAACCAAAA
CCAAAGTGCCGATAAAGGCACGCCATGGGTGATCAGGCCACGCTTCCACT
CAAACGGAGTGGGCAAGTTTTAAAGACTAGTCTTACTAAGTTTTAGATGT
CCAGACCGGTGAAAACCTTCCTGGTGTTCAGCCACTGCGTTGCTCCTCTA
TGGGGGCCTGCCACACACTGCCCTGGCAAGGCGTCCCACTGGGGCAAATG
CCTACCCCTGAGCGCTCTCAGGATCTGCGTTGCTTGGGCTAGTTGGAGTC
CCCCGCCGAGATGTTGCACAGGGCAGGCTTAAGCCGCCTAAGGAGCTGCC
TCGACCATCCGCCAATCACCTCGCTTCCCGGTCAGGGAACCAAGAAATGT
AGCAGAATGAGCCGCAGACAAAACTCCTCAGACACCGAGTTGAAGAAGGA
AGGGGTTTATTTGGCTGGGGGCATCGGCAAGACTCCTGTCTTAAGAGCCG
AGCTCGCTAAGTGAGCAATTCCTGTCCTTTTTAAGGGCTCACAACTCTAA
GGGGGTGCACATGAGCGGGTCATGATCCATTGAGCAAGCAGGGGGTGCGT
GACTGGGGGCTGCATGCACCGGTAATTAGATCGGAACAAAACAGGATAGG
GATTTTCACAGTGCTTTTCTATACAATGTCTGTAATCTACAGATAACATA
ACCGATTAGGTCAGGGGTCGATCTTTAACGACCAGGCCTAGGGTATGGTG
CCGGGCTGTCTGCTTGTGGATTTCATTTCTGCCTTTTAGTTTTTACTTTT
TCTTTCTTTGGAGGCAGAAATTGGGCATAAGACAATATGAGGGGTGGTCT
CCTCCCTTAGTGGGAACAGCGGAAACTCAACCAGAGGGTCTGTCATTTGT
CTAGGTTCCTTGTTGCTGGACAGACTAACCAATCCACTTTGGACATTCCT
GCAAAAACCCACATACCATATAGTTTAGGGGAAGCGGATAAAGCCAGAAG
ACTCAGATTTAAGGTCCCATCCCAGCCACCTGCTAATGGGATGATGCAGG
GAAGCATTTCTCACCTTTTCAGGGCCTCAGCGTCCTTACCTGCAAGAGCT
GGTAATTGACCAATATCCAGCTCATGGGGCTCAAAAAGGATTATGTGGAA
TGAGGCATAGGAAAAAGGCTTTGTAAACCATGCTGTGCTGTCAGGATCAG
TACCCATCTGAGATCAATTCTTGCTTCAACAAATAGGATTTGTTCACATT
CAAACATTTAGGAACAATTGTTCCACCAATTCCGTGAGGCATAGTTATTA
GTTAAGAAAAACCACATTTAATAATAATAATGATGATTAAAATAATAGTA
ATAGTGGCTAACAGTATTTGAGCTGTATGAATGTACCAGGAACTATGGAA
GTACTTTATCTATATTTAATCCTCATTACAACCCTATCAGGTGGATACTA
TTATTGTCCTCATTAATAATGAGGAAAACAAGGCATTGTGAAATTGTTCA
CATAACCAGAAAGGGGTGGACTATAGAGGAACCAGGTATGTGAGACTTCA
GAGTCAAAGACCCAAACCCCTGTTCAAGCTGGCCCTCACCATCAACACTA
TGCTGTTTAACACTGGTTTAGCAGGGGTGTGCTTATTCCCAGTGGCCAGT
GGTTTCTCTGGGCCCTGTTGACACATACATCCCAGCTGTGACCAAGGGGA
GCAGCAGTGGCTTCAGGTGGATGCTCCCAGTGGCTAACGGGCTGATACCC
TTTCCCTCTTCATAACTCTCATCTCCAGAATGTGGCAGCTTTCAGGTGGG
TTTTATGTGCCTGCTCCTGCAGTAGTAAACCAGATTTAATCTAGAGTCTC
TGTTGCCTCTAGCTTACAGGAATCTTAAGAAGCAGAGATCTGCTCCACAA
TCACTGTTCAGCATTTGGTGTCCAGAATGCTAAATTAAACAGGTTCATTT
AGTAATCCTGAGGGTGGGGGTGGGAGGCAGCTTTTGCAGCTGACTTATCC
TGGCGGCTTCAGGAAGTTCTGTTTCTCCTTAAAATAAAACTTGCCCTTGG
GACCAAACTGAGGTTATCTTATAGAAGGAGTGGTTGATGACTGCTGCCAA
ATTTCAGGAAATCACACACCCAGAGCTCCAGTAGTGAAGTAATGTATATC
CAATCAGTGAGGGAACATCCCGGTGGAGACTGAGGGCTGGAGAATAGTGG
GAAGAAGGGAAAGGCAAACCTCACAGCTTGGCTTCGGTGGCCCTGGTGAC
TTCTGGCCTGGGAGGACCAAAAAATGGGGGGAGAATTACTTTGTCTTGGC
CTATGTCCATCACTTCCTGAGGCTTCTGACCCCCAGCAATGGGGAGGCAT
CTTGGGCTTGCTTAACTAATCAGTTTCCAGAGCAAAGTGCTCTATCCCAG
CAACCAGATTCATGCTCAGGAAAACACTCTCAAGAAAATCCCCTGAACCC
AAGAATTCCTTTACCCACCCAGTGAGTTAAGATATGCCCACTCCTCCCCA
GGGGTGGTGTTTCTCTAACATTGAAGACAACATTGTTCTGTTCCTAAGCA
TCTGCCTCCTGAGAGGCCTGCTTCCCCTGCTTGTAGGGAGAAAGGAAGAG
AGAACAACGGGAACGGGTAGGATCAGGTGGGCCATGGGTCGCTCAGACCT
CCTCTTCCTGGATGATGCTGAAAGCCCTCCTTAGCTTTCAGTTTGTCTTC
TGGTGGTACTCACATGGAACAAATGGAAGAGTGTTGTATGTTTCGCAGTA
TAGATAGCTCATTTCACCTCACCTCTTTCTGATCACTGTCTTTTGTTGTG
ACAGTGACTACCTGTTTTTTCCATTCTGAGAGATCCAGAAGTGATGTTGG
AACGTTGAGATGTGCACGTGGCTTCCCTTTGCCTTCTTTTGTCTTGCTTG
TGTAATAGACCAGGGTGTATGCAGAGGAATGGAGGGGGTCCCCTGGCCTC
GCCCCTCTTCATTCTCCCTCTTCTCAACCCCCACTTAGTTTTATCCTGCT
CCATGTCTTTAACCAGCCCCAGCACTCACTTCAAGCCCCTTTCTGCCTGT
GGCGGCGCTGGCCTTCAGACCATTTATGTGCCAGAGCCTGAGAGCCAGTG
ATGGTGGAACTAAGCAGCCAGGGATACTCCCAGGGCAAATGAGCCCATCA
GGAAGCCTGCACAGGGCCAGGCTAGTTTGGCCCCTGTTCTGAAGTTAAGT
TCACTAACATGTGCTTGGCCATCCCATTCTGTTTCTGTTGAGGAAGGACA
TGAAGACACAGCTGTGTGAATCTGCACCTGTCTGTATCCTGGAGGATCAA
CGTGGATGGTTATCACTGCGGAATCTGGGGCCTCATGAGCCACCATAGAG
AAAGTCAGCTGAGTTCCTAGTGCCAGACAAAATGATTGATTTAGAATTGA
AAAAAAACTTTTTATTTTATAGCAATTACAGATTCATAGGAAAAATAGTG
CTTACAGGTCCTGTGTAGCCTTCACCTAGTTTCTGCCAGTGGTTGCATCT
TATATAATTTCAGTATAATATCAAAAGCAGAACATTGACATTGCTATCAA
GTGTGTGTATAGTTCTGTGCCATTGCATCACACATGAAGATTTGTGTAAC
CACCGCCCCACCACTTCAAGATACAGAACTATTCCATCACCACGAGGATT
TCTCCTGTGCTGACCCGTTTATAGTCACGTCCCCTTGCTTCCGTGACTAC
CATCTCTAACCCCTGGTAACCACTAATCTGTCCTCCACCTGTATAATCTT
GTCACTTTGACAATGTTATATAAATGGAATCATACAATCTGTGACCTTTT
GAGATTGGCTTTTTTCATTCAGCATTATGCCCTTGAGATCCATCCAAGTT
GTTTTGTGTATCAATAGTTTGTTCCTTTTTATTGCTGAGTAATATTCCAT
GGTATGACCGTACCATAATTTGTTTAAACGTTCACTTATTGAAGGACATT
TGGGTTGTTTCCAGTTGGGGGCTATTACAAATAAATCTGTTATGAACAAT
TGTGTAAAGGTTTTGTGTGGACATTTTCATTTCTCTAGGGTAAATGCTCA
AGTGTGTTTGCTGGGTTATATGTTAGTTGCATGGTGAGATATTTTTTAAA
AAACTGCTGAACTGTTTTCTGGAGTGATCACACCAGTTTACATTTTCACC
AGCCATATGTGAGAGACCCAGTTGCTGTGACACCTCATCAGTATTTGGTA
TTGTTACAAATTTTTATTTTAGCGATTCTAATAGATATGAATAGTGACAC
CTCATCATGGTCTTGTTTTGCATTTCCCTAATGGCTCGTAATATTGAACA
TCTTTTCATGTGTTTATTTACTGTTTGTATTTCCTCTTCATTGGAATGTT
TTTTCCTATATTTTGCCCATCTTCTAATTGTAATCTTTTTTTTTTAACAG
TTGCATTTTGAGAGTTCTTTATATATGTTAGGCACAAGTCTCTTGTGGGG
TATGCAGTCTGAAATGATTTTCTTCTAGACTATAGCTTATTTTTTCATCC
TCTTAACAAGGTATTTTACAGAGAAAATGTTTAATTTTGATGAATTTTAA
TTTATCAATTTTCAATTTTCTGGATCCTGCTTTTGGTGTCATGTCTAAGA
ACTCTTCACCAAACCTTAGTTTCCGAATGATTTCTTCTATATTTCATTCT
AAAAGTTTTCTCATTTACATTTAAACTTATTTTCTATTTTGAGTTAATTG
AATGGATGTAAGGTTTAGGTTAAGGTGTTTTTTTTGTTTGTTTTGTTTTG
TTTTGTTTTTTTACCTATTGCTATCCAGTTGTTTCTCCACCATTTTTTTG
AAAAGACTATCCTTTATTGAGTTGCCATTGCACCTTTGTCAAAGATCAGT
TGGCTGTACTTGTGTGGATCTATTCTGGGTTTTTTATTTTGTTTCGTTGT
TTCTGCTGATACCAGTCTGTCTTCATTACTGAAGCTAGAGCAAGTCTTTA
TTTTTATTTGATGAGCCCATCCATCTTTATTTTCTTTAAAAATTTATTAT
TTTTGATTGACAAGTTTTTGATTGGCATAATTGTATACATTATGGAGTAC
AATGTGACGTTTTAATCTGTGCAATGTGGCGTGATTAAATCAAGCTAATT
AATGTATCTTGACCTCGCTTATCTATCATTTTTTATGGTGAGACATTGGA
AATTTACTATTGTTTTGAAATATATATTACATTATTGTTGACTATAGTCA
CTCTGCTATGCAACAGATCTCAAAATCTATTCCTCCCATCTCTCTGAAAC
TTTGTACCTTGGATCAACCACTCCCCATTTCCCCCCTCCCACCCCACCCC
CAGCCTCTGTAACCATCATTCTACTTTTTACTTCTATGAGTTCAGCTTTA
TTAGATTCTGCATTTAAGTCAGATCATGAGGTATTTGTCTTTCTGTGCCT
GTCTTATTTCACTTAGCATAATGTCCTCTAGATTTATCCATGTTGCAGCA
ACTGACAAGATTTCCCCCTTTTAAAAGGCTGAATAATATTCCATATGTAT
ACATGCCACATTTTTCCTTACCTATTCATCCACTGATGGATGCTGAGGTT
GATTCCATATCTTCGCTATTGTGAATAATGTAGAGCAAGTCCTGAAGTTG
GATAAAAGCTTGGTTTTATTGGGCAATGGTATGTGTTTATCTTATGGATA
CCAAATTTTATTACCTGTTCAGTTTTTCTTCTATTCTGGCTTTTAGGAAG
ATTTAAGGACAGGTTTAACGCTTGCTTTTGGTAAGTGAGGGGATCTTATG
TATTTATGTGCTCCAGCTTTTCTTCTGCCCTTAGCTGATTGGTATGTTTT
TTCCCTTGAAGTTGATTTTCAAGTCTATGTTCCTAAATTTTTTCTAATCT
TAATGACAGCCCTGCAAGAGAGGTATCAACGTCTCCATGTCACAGTTGAG
GAAACCAAGGCTCATCCAATGCCACATATCCAGTAACTGGCAAAGCAGGG
ATTCCCACCTGGTCATCTCACTCTTCTTCTTCCCGTGACCACGGAACATC
TCTCAGCTCTGGATTCTCTCATCCACCAGCATTTGGGCCTGAGCTCAGTG
ATCTCTGAGGTCCTTTCAGACTCTCAGATTCTTATCTCATGATGTTAAGT
TGCTGATTGAGATGAGACTAGGAGCAGGGCCAGTTAGGTCTGGACTCTTC
TAGGAGACCTGTTATGAAACCTGGCGAAAGCACCCGCATTGTCTTTGAGT
AATCCCCAGCCCTTTCAGTTGAGAGGACCTTAATTCCCTGGGTGTGTCTT
ACTGTTTGGGATGGATTAAATTTGCCCAGGGGTGGAGATGAAACCCAGGC
AGGAAGTCTGAGACTGCACTGAGGGGTTGAAGGATGGGATGATACTCTGC
AGCCAGCAGCTCTCTTCCTGAGCCTGTCTTTCCAGCCTCTTTGCCCTGCT
GAAGGGAAAAGCTATGGAGGCTCACATGGATACACTGGAGGCTCAGGCAC
AGCCAGAGCAGTAACCCCAGGACTTGTGACCTTCGGAACACTCAGTCTGG
TCAGCATTTGGCACTGTCTCCCACCCCCGAACAGGGCAGACCCCTACCTT
CAGAAATGTGGCAAACTACTGGAATCCTGTAGGAACCTGAGAGTCCTGAT
GACAAGTTTCATTTTGTTTACCCAAACATCAGTCATACTTGTTTATGTCT
AAAAACTGCTCCATCCAACACCCTTGAGAAATGCTGGCCTAGAGCACAGA
CAGCCTCAGTTAAAATGCCTGTGTTATCAGAGAGGGCTACCTTGAGCTAC
TGTTTATCAAACTGCCCCACCAAGAGGCCCTGAATTCTAGAAGGGAATGA
AGAAGTTTCCATGTAGGGGTAGGACAAGGTGCGTGAGGGGAGATGTCTGG
GGATGTGGCCTGACTCCTGAGTATTACATTTCTTTGATATATTTTTATAC
TTTAATCTTAAAATCAAATGAATGATATATTGTCCTCCACAATCTATGTG
TTTCCAAATAATATGTCATATTTCTCTCCAAACCTTGGAGTAACATTGAT
GCTCTAGGAAGATGTGTGTGTCTAACTTGGCTCTCCCAGATATGGACTCC
CAGGTGGAGTGAATTGAGTTTGGGAAATACAACACAGGCAGACCTCACAT
TGGAACTAGTTTATCTGGTTGACTTACCCACCCAGCTTGTCAGGTGCAGA
GGGAATGGTACCTAGACAATTTAAGTGGGACAAGTGTGTGAGGTGTGGAC
ATCAGAAAATTCCTGGGCCGGGCCCCATGGGACACCGTCAGACAGAACCA
GCTAGAGAGCACAATGAGGCCCTGTTGGTGGGGGGCAAAGGGACAGCAGT
GGAAGGGAAGGGTGGGGAGAAAGCAGGCTTCCAGAGTCACACTCCTGCTG
CTCGTTTGGCTGCCAAAGAGATTGCAGAATAGGATTAAACTGATCTGTTT
CTCAAACTGTTTTATAAGTCATTCTTCACTTGTGCCACAGGCCCTTTTGA
AAACTTCCTCCTGTAACTTTGATTTACAGTGTATTTGGGGTATGTGTCTG
GGAGCTGAGACTGATATGGAGCTAGGACCTCACTCACTCAGTGAGCATTT
ATTGAGTGTCTACTGTAAACATCTATTGAGTGTCTACTTTGTAAGCATTT
ATTGAATATCTACTGGGTGAAGCTCTTTACTAGGTGTTGCTGTTTATACT
TGGCTCCCTGAAAGTCTCCCAAGTTTCCAAGAAAAAATTGCCTATAAGTT
CCCTGCCTCCCAAGGTGACCAAATGTCTTCATTTGCCCAGGACTGAGGGG
GTTAAGACACCAGACTTTCAGTTTTGAAACTGGGAAAGTTGTGGGCAAAT
TGGAACGAGTTGGTCACCTCACTGCCTCCCTTGCTATGACATTTTACAAG
AACAAACATGCACCTTCTGACTGTGGTGCAGGGGAAGTGCCGAGGAGAGT
CAGGGATGGTGTCCAAGAGTGGAAATGAACCTTAGGGACCTATGGGGAGA
AGAAATGGGGGCTCCCAGAGAGCAGGCAAGTGACAGCCAGGATGGCAGTT
GCTGGGCTTCTGCACAGCGGTGGAGCTCATTTCCCTCTCTGGTTTCTATG
TAGAAGTCCTCCCACCTAGCTTCCCACCTTCATTACTGGCCATGATTTTT
TACAGTGTTTTTCAACATCTTATTGTATAGAATCTATATAAATGTTGATA
AAGCTGAGCTATTGAAAGATGGTGCTTCCTGGCTTATGGAATTATTGACT
TTCAGACTAGACAGTGATGAGAAAGCTCATCTGCTCAAAGACGCAGGAAT
CCTCTTGATGTCCTTGCCTCTGCTTGCAGAGTTCTGATGGGGAGCTCACT
GTAGCACAAGGCAGACCCAGAACATTGTAGCACCCCAGGCATGTACCAGG
TTTCTCCTGTGTGGAGCTGAAATCTTTACCTGCCCTTCCATGTGTGTTCC
CCAGAGTGAAGCTTGAAGCTAGCAGAACCCATTACTTCCTGGTCACTTGC
TTCACTGTTTCTAGTACAATACTGTCTCCCCTCTCCCAGAAGCCCCTCTA
ACTGTGGACTGCTCAGCAGGGTTGAAATAGAAAACCAGGTAGTCAGTGCC
ACCCTCCTCTGAAAGCCAAACTAGGCTCTGAAGGTGGGCGTTGCTGGGAC
ATGTGTGGCCCCCAACTCCTGATGTCCCAGATCACAAAGGTTTCATTGCT
GGTTCTGGTCACTGACAGGAGCCTGGTCACCACTCATGTGAGTGTCTCAG
GCTTTGTGGCAGCAGCTGGAAAGTGGCCCAGCAGAGACCCTCCCATGATG
CCCCCTGCACTAGGCCTTGCAAGTTTGTGATGCCCTCATCAGCCCCCAGA
TCTGTGCCCACGTCATCTCTCACACTCCTCCTTCTTACCCCCGCCTCCCT
CTGTTCTCACACCCCAGCTCTCACTGCTCAGCAACTTTGGAAGCTTTCAT
GGAGTATCCTAGACAGGTCATTGACTTCACCCTCCAGCCTCTAAAAGGAC
AACATCTCTCTGGAGGAAGTAGAAAAGGGGCTCACTGGAGACAACATTCC
CTGGCTTCATTGCCAGAGGGCTAACAGAGACCCCTCACACAGGGCATCTC
ACCCAGTGCTAGTGGGAACGGGTCTTATGGGTCCCAGCCTGTGCTCCAGG
GCTGCACCCCGCAGGGTGGTATGTGGCAGGTCCAAGTTAATGAAGAACCA
GCCTTGTATATGGCAACTACTCCACCTCTGGAACGTGTCCCTGACTTGGA
AAGTGAACTCACAGGGCTGTGGTTGCCAATTTCATTTTCAAATTGCCCTT
GATATCAGGACGCTGCCTTCCGCACAGCTGAGATCGTCCTCACAGCAGCT
CAAGCCTCCTTCTTCTTCAGTCCTCAGAGTCCCAGGACTCCCACATCATG
GGCCCACTGAATCCATGTGTAAATTTAATGTGTAGGCTGGGCACAGTGGC
TCACGCCTGTAATTCCAGCACTTTGGGAGACCAAGGTGGGCGGATCACAA
GGTCAGGAGTTCAAGACCAGCCTAGCCAATATGGTGAAACCCCGTCTCTA
CTAAAGATACAAAAATTAGCTGGGCATGGTGGTGCACACCTGTAATCCCA
GCTACTTGGGAGGCTGAGGCAGGAGAGCTGCTTGAACCCAGGAGGCGGAG
GTTGCAGTGAGCCGAGATCGCGCCATTGCACTCCAGCCTGGGCAACAGCA
GAGCGAGACTCCATCTCAAAAACAAAAAACAAACAAACAAAAAAAACAGA
CAAAATTTAATGTGTAAATGTGTAAAATTTATAAGGACTGGGGTTGGGCA
TTAGTCAAGCCTGTTTTGATTGCCTTAAAATCACAAATATTTAGAATTCC
CACCTGATATGGTTTGACTCTGTGTCCCCACCCAGATCTCATCATGAATT
GTAAGCCCCACATGTCCGAGGAGGGACCTGGTGGGAGGTGATTGGATCAT
GGGGGCAGTTTCCCCCATGCTGTTTTTGTGATAGTGAGTGAGTTCTCAGG
AGATCTGGTGCTTTTTTTTTTTTTTTTTTTTGGACAGAGTCTCACTCTGT
CACCAGGCTGGAGTGCAGTGCTGTGATCTCAGCTCACTGCAACCTCTGCC
TCCCAGGTTCAAGCGATTCTTCTGCCTCAGCCCCTTGAGTAGCTGGAATT
ACAGGTGCGTGCCACCATGCCCAGCTAATTTTTGCGTTTTTTTAGTAGAG
ACAGGGTTTAACCATGTTGGTCAGGCTGGTCTTGAAGTCCTGACCTTGTG
ATCCGCCCACCTTGGCCTCCCAAAGTGCTGGGATTACAGGCGTGAGCCAC
CGCGCCCGGCCGAGATCTGATGCTTTTGAGGTGTTTGGCAGTTTCCCCTT
TGCACTCTCACTCTTGCTCTCTCTCCCTCTCTCTCTCTCCTGCTGCCTTA
TGAAGATATGCCTTGCCTCCCCTTCACCATCCGCCATGATTGTAAGTTTC
CTGAGGCCTCCCCAGTCATGTGGAACTTTGAGTCAATTAAACCCCTTTTG
TTTATAAATTACCAAGCCTCAGGTAGTATCTTTATAGCAGTGTGAAATGG
ACTAATACACCACCCTTAGCACCAACACATTTTTAGCAGCAATTATAATT
TTCTGAGCTACTATCACAAATATGGGCACATTTCATTAGGACACACATGT
GGTAAAACATAGGGTGGTGTATGGATATTTAGTTGTGAATGCAAATACAC
TTGAGGCCTCTTCCTGGGAGCACCCATTATGTACCAAATTTACTGAGATA
ATATGGACAAGGGTAGGTCCTTTCAGAAACACTGACCAAACCCTCCCTGC
CATACTGTGAGGTATGCATTGCCCACATTTTATAGTGGAGGACATTGGGA
GGCAGCCCATGGGAATTGCCACATTCCACAGTGACAGATCTGGAATTGGG
GCTCTCTGGGAGTCCCCGTCTTAAGATTTTTTCTCTATGTACTTCACCAC
AAACCACAGACTGTTGAGCCAGACTTTGCTATTTGATTGCTTTATAACAA
TGGACACACCAGAACTGCCTATTTATATCTGTTGTTGTATTTTCCCAAGC
CAAATTCATCAGTGGTGGTCCTGAAGGTCTTCATTCCTCAGTTTCCTCTT
TCTTTGCGCATTCCCTAACCCATGGTTGTAAGCCTAGACGCACACTCCAG
CTGTATGGCACATGAGCATTTACGCCCTTCATGGGCGTGGGGCAGTCAAG
TGGCTTAAACTCTTGATTTCTAAGTGAAAGAATAAATGGAAACCCAGTCA
GCTGCATAATGCTTTATCATATGACTCACCTTCTCAGAAACTTACAGTGA
GGAGGTGACTTGTATATCCAGCAATCAGAGAGTCAATAAATAAATCATGG
GACATCCATCTATAAAATACATGCAAATCATTTTTTAAAAAGTCTTATGG
AAAATAGTTGAAATTTGGGGGAAGAGTTCATTCTCTAATGTTAAGTAAAG
AAGGCAGGATGGATAAGCACATGTAGAGATGATCCAAATTTGTCAAAATC
ACAGTATACAGAGAAGGAAGACTTGAAGGGCACGTACCTATACTGTGTGT
GAAACCTATAAATGGTTTTGTTTTTTGTCTTTGTGAATTTCAGTGTTTTC
CATTTTCTACAGTAAACATGTCCTGTATTTATGCTTAGAAAAATCTATCC
ATTAAAAAGCCCTACAGTCTGTGGTCATGCCCCAGCTTCCTTTGGCAGGC
ATACCAAGACATCTCATTGTGGTATGTAAAATGTGATGTACGATGGAAGT
GGCTCTTCTCCGATTACCTGAGGGTGGACCAAGGATGCAGCTGGGATTGC
AGCCAAGAGGATGGGGTTGTCTAGAAGGACTGACCCCTGCAGTGCCATCA
CTGTTCCCACTGCATTTTTCCATCTGTTGTCACAGTTGAGTCATGGCTTT
CCAGAAATTGAGGCAAAGTCAGATAACAATACTTGAAGTCTTAAAGGGAG
GCAAGAATGTGGAGCCTTTTCCTTTCTGGGAAGATCAAGAAGTGATTCTG
TCCAGGAATCTGCTCTCTCTGCTCTTCTGGTGCCCACTCCCAGGGGCTGG
CTCAGAGTGTAGGAATTCAGCCCTAAGGTTTGGCCAAACATCCTGGAGAA
GCAAATGTATCTGATTGGCTCCTTGCCATGTAGGAAGAGTAGGCCTGAGG
GTGAGGTGGTGAGGACAGAAGCCAGGATGGGATCTGCTAGGTGCTAACTA
GGGGTCTGTTCCTGAGCCTGGGGTTTTCTGCCTGGGAGGATGTAGAGAAC
ATTCTTGGGTGGGTTTTCACCCAGTTGTTGGTGAGAGTTTATTGACCATC
CACTGTTTGAAAGGTAAAACTCTACTAGTGTAAACAGCAGGGGTTTCTGA
ATTTTGAGGCCAAGACTGGTGTTGGGATGGATGCTGGAGACCCATTTATT
CATTCATTTATTCTTATTGACCTCCTGTCATCAACCACTGGAGATCCGAT
AATGAATAAAACAGAGTGGACTATGCCCATATGAGTGAGATAGACAGTGG
ACAGAGCACAAATAAATACAAAGTCACAAAGCATATCATATGATAGCAAA
AAGGATTTGGTTTTGTGAGAGAGAATGGGGTAGAGACATTACTGGATTAT
ATTTAATGCATCTGGGGTCATGGACTGAATTTTAACAAGTCACCTTCACG
GTTCTCATACAGAACCTCCGTATGGGGAGCACGACTGTGAATCAGACAAG
GTCCCTATCTTCCTTCTAAAGGCTGTGGTTAGGTGGGGTAAGATGAACCA
TAAGCAGCTATTGTGAATTATGGTTTCCATCAACATGACAGGTTCACATG
GAATCCCTCCTACTACAAACACCACATAAAATAGAACAGCACCAGAGATT
TAAATACATGGCAGAACTTGCAAGAAAGAAAGTGAAATCTTTAGGTGAAA
GAAATGAAGAATGAATTGAACACAGGAACAATGAGGACATCAGTTGACCC
TGCACAAGGAATAAGGAGGAAATATGTGTGGTGTGATTAGACCAGTAAAG
AGATGCTAAAGGAACTCCTAATGGATGCTGGAATTTAGTACTCACACAGG
TACTCAAAGATGATAGGGGAGTTGAAACTGAGACCCATTCATAAAGCTAG
GTGCCTTGGTGAAAAGGGAACTAATAAAACCCTGCCCACTACTCCAGGGT
CAGCAGGGGTTTGTCTCTAACAAGATCTCTGGGTAAAAAAATTTCTCTGA
GAAACTAAAACCCCAAGGCTATATCACATAGAATCAGAAACAGAATTTAT
GCCATCCATATGGTATAGTGATGCTTACTTTATGCCCTATATAGAAATTA
ACTTCAAATGAATAAAAGATCTAAATGTAAGACCTAAAACTATAAAACTC
TTAGAGGAAAACACAGGGAAAGCTTTATACCATTGGATTTGGCAAGGATT
TCTTGGATATGATACTGAAAGTACAGGCAACAAAAGTTAAAATAGATAAA
TTGTATTACATCAAAATTTAAAACTTCTGTGCATCAAGTGACACAATCAA
GAGAATGAGAAGGCAATCTACAGAATGGGAGAAAATGTCTGCAAATCATA
TATCTGATGAAAGATGAATATCCAGAATATATAAAGAGTTCTTAGAACTC
AACAACAACAAACCGAATTTAAAAATTGGCAAAGGACTTGAATAGACATT
TCTCTAAAAATGATACACAAGTGGCCAAAAGGCACATGAAAAGATGCTCA
ACATTGCAAATCATTAGAGAAATACAAATCAAAACCACAATGTGACATCA
CACCCATTAGGATGGCTACTATAAAAAAACAAAACAACAGAATATAACAA
GTGTTTTTGAGGATGGAGAGAAATTGGAACCCTTGTGCACTATTGGTGGG
AATGTAAAATGATGCAGCTGCTATGGAAAACAGTATGGCAGTTCCTGAAC
AAATTAAAAGTAGAGTCATAATATGATCTAGCAGTTTTGCTTCTGGGTAT
ATATCCAAAAACATTGAAAGCAGGGTCTCAAAGAGATATTTGTGCAGTTA
TGTTTCTAGCAGCACCATTCACGATAGCCAAAACGTGGAAATAACCCAGA
TGTCCATCAATAGATAAATGGATAAACAAAATGTGTATATACATACAATA
GAATATTATTCAGCCTTAAAAAGAAAGGAAATTCTGACACATGCTACAAT
ACAGATGAAACTTGATGACATTACTCTAAATGAAATAAGCCAGTCACAGA
ACGACAAAATACTGTATGATTCCATTTACATGAGATACCTAGAATAGTAA
AATTCATAGAGACAGAAAACAGAATGGTGGTTGCTAGGAGCTAGGGGTAA
GGGGAAATGGGGAGTTATTGTTCAAGGGGTATAGAGTCTCAGTTTTGCAA
GACGAAAAGAGTTCTAGAGATGGATGACGGTGATGGTCGCGCAACACGTG
AATGTACTTAATGCCACTGAACTTGAGATAGGCTAGGCAGTGGTGGCTCA
TGCCTGTAATCCCAGCACTTTGGGAGGCTGAGGCAGGAAGATTGCTTAAG
CCCAGAATTTTGAGACCAGCCTGGGCAACAAAGTGAGACCTCATCTCCAC
AAAAAATTTAAAAAAATAGAAATAAAAAAATTAGCCAGGCATGGTAGCAT
GTGCCAGCTGAGGTGGGAGGATTGCTTGAGCCTCGGAGGTCAAGGATGCT
TTTAGCCATGATTGCACCACTGCACTCCAGCCCAGGCAAGACTCTGTCTC
AAAAAATAGTATTAATAAAAAAGGGTAAGATGGCAAGTTTTATGTTACAC
ACATTTTATCACAATTTTTAAAATATGTGGGTAATCATTAAGACAAAAGA
CATTGAATATACCTTCTAAGACAAAAGAGAGTAAAGAATAAAGAAACCTA
ATTAAATAATAAAAGCCTAGAAAAGGAAGGGGAAATGGTTAAAAATAAAA
TAAAATAAAACAGCCAAAAATAAACTTCATGGTAAATAGGAACACAATAT
AAATATAATCAGGTTGATACAAATCTAGATTTATCAAAAATTATAATACA
TGGGCCGGGCACGGTAGCTCACGCCTGTAATCCTAGCACTTTGGGAGGCC
GAGGCCGGTGGATCACGTGGTCAGGAGATTGAGACCATCCTGGCTAACAT
GGTGAAACCCTGTCTCTACTAAAAATACAAAAAATTAGCCGGGCGTGGTG
GCAGGCGCCTGTAGTCCCAGCTACTCGGGAGGCTGAGACAGGAGAATGGC
GTGAACCCGGGAAGCGGAGGTTGCAGTGAGCCGAGATTGCGCCACTGCAC
TCCAGCCTGGGCAGCAGAGCAAGACTCCGTCTAAAAAAAAAAAAAAAAAA
ATTATAACACATGAAAATGGACTACTCATTATTTCAAAGCTACAGCATCT
CAGATTGGATAAAAAATAGAGATAAAATCTAGTTACATGCTCTTTAAAAG
AGACATCTAAAACAATATGACACAGAAAGATTGAAAATCAAGAAACAGAA
GAAGATGTAATAGAAGAATGCCAGCTAAAGAAAGATGATTTATCTACTTT
AATAACAGATAAGAATGTTTAAAATAAGAGTGTTTAAAGCAAAAAGAATG
CCAGAATGTGAGATGTTTGTAATGTCTAAAACTGACGAGGGACGAATATA
CAGAATGCACAAGGAATCTTGATAATCTACAAGAAACCTAATAGGAAATG
TGTGAAGGATTTGGACAGGTGGACTGGCATGCCAGCTGGGCTCGCCCTAC
CCTCTATACTTTTCTCTGAATGTCATGGTCATTGTTTTCCACTGTAAACC
TCTGTCTTCTCCATGGCTGAGTCATGCTTGGCAACTAGAAAGTGCCTGTT
TTCCTTCACTGGAAAGCCCCCAATTTGACAAATTTTCTGTTAGTCTCCTA
TTGCTGCTGTAACAAATTATCACCACGGAGTGGCTTAAAACAACACAAAT
TTATTGTCTAATGGTTCTGGAGTTTGGAAGTCCTTGATTTCTTAAGGTGT
TGGTAGGGCTGTGTTCCTTCTGGAGGCTTTAGTGGAGGATCTCTGTTTCC
TGGCCTTTTTCACTTTCTAGAGTCACCCACATTCCTTGGCTTGTGGCCCC
TTCCTCCATCCTCAAAACTAGCATGTCACCATCTGAATCACTCTCCTTTT
CACCTTTTTTTGTTTTGGACCCTTGCCTCCTTATTTTAAGAATCCTTGTG
GCTGGGCGTGGTGGCTCACACCTGTAATCCCAACACTTTAGGAGGCTGAG
GCGGGTGGTTCACCTGAGGTCAGGAGTTCAAGACCAGCCTGGCCAACATG
GCGAAACCCTGTCTCTACTAAAAATACAAAATTAGCCAGGCATGGTGGGG
CACGACTGTAGTCCCAGCTACTCAGGATGCTGAGGCAGGAGAATTGCCTG
AACCTGCGAGGCGGAGGTTGCAGTGAGCTGAGATCATGCCATTGCACTCC
AGCCTGGGCGACAAGAGCAAAACTCCGTCTAAAACAAAAACAAAACAAAA
AAAACTCTTGTGATTAATTTAAGACCACCCAGCTAATCCAGGATAATTTC
CCAATAAAATCCTTAATCACATATACAAAATCCCTTTTCTCATGTAAGGC
ATTTATTTACAGGTTCTGGGGATTAGGATGTAGACATTTGGGGAGGCCAT
TATTTTGTCTAACACATGGTTCAACCAAACCAGAGACAGTAGAAGATACA
CACACACACACACACACACACACACACACACACACACACGAAGAGATTCA
TTACAAAATTTGGCTTACATAATTTGAGGAGCTGGCTAAGCAGCTTCAAA
ATTCACAGGGCAGGCAGTCAGGAAGGGATGATCCCAGGCAGGCTGGAACT
CACAAGCACAAAAGCTGCTGTACACAGGGAGAATTTCTCTCTCTTTTTTT
CCCTCTCTCTCCTTCTGTCTGTCTCTCAACCTCTGCTTTAAAGGCCTTCC
AACTGATTGAGTCTGGCCCACCCAAATTATCTAGGATAATCTCCCTTACC
CAGTGTCAACTGATTATGTATTTTAATTACATCTGCAAACTCCCTTCACA
GCAACACCCAGATTGGTATTTGAATAACTGCAGACTGTGGCCTAGCCGAG
TTGCCACATCAAAAACACTGCCATGGGGGCCAAGGGCAGTGACACTCAAG
ACAGGTATAGGACAGAATGTGGCAGCAGCAGGCCAAGGGCTCCAGGAATG
GTGAGCATCATTGAGCAATAGGAAACCCCAAATGGAGGATGCAGGGAGCA
GAGGTATTTTGTTGAAGTCACTGATGAGAGGAGGAAGGCATGCTTGGGGT
AAGCTTCTGCAAGGAGACCTGGAATGGGGCATTCTGTCCATGCCTTCCCA
CAGTTTGAAAATCCCATGAGGGCTCTGCTTCTCTCCTATACACAAATTCT
GCCAGAGCCATAAAGATCAGACGCCCAGCGTATCACCAGTGTGTAGATGT
CCGTTCCTTATGTGGCTTACCAGTATGTGTCCTGTGACCTACTGCTAAGG
TCACAGGCCCCTGGGCTGCAGACGTGAATCCACCCATGTGCTGGAAAGGT
AGGTGAGCTCATGTCTGCTTCTCCTAGGGCTGCAACTGGTAGGTGTCAAG
TGTTTGCTTAGCTAATCTTTCCTCATCTAAAGATGCTTATTGATGATTTC
ATTCAGGAGTCTTGAAACCTACAGGTAGAAGTCCCAGACAAAACAAACCA
GGTGTAGATGATGAAACCACATTCCCTTGCACTTGCCTTTGAGAACTCCA
GGTGAGGGAAGCGGAATGTTCAAAGTAGTTGGGAAGCGGATCAGTCCTGG
AATGCTCCTGGCCCACACCGACAGTGCTGCCAGACCCTCCGTGTGGAGTT
CCCATGCCCAGGTGGGCAGATGGCCAAGGGGAGTACAGAGAAATAAAGGA
ATTGAATCAATTTTACCCCAGAGAAAGACTTTTTTTTTTAGCTGCGCTGT
TTTATGCAATAGATTTGCACAAGAGGTTTAGCCTTGTTTCCAAGAAAGAG
TTATCAACAAGGAAAGATTTAGGTTAGAGCTCAGGAAGACCTACTTGGGT
CTAAGGTGGTTGTTCTTAAAGTACGGTGCCTGGAGTAGCAGTGTTATCAT
CACATGAGAGTTTATTATAAATGCAAATTCTTGGGCCCCATCCCAGACCT
ACTGAATGAAAAACTGGAGGTGGGGCCCAGAAAACTGCTCCAACAAGCCC
TCTAGGTAATTACAATATACCCTAAAGTTCAAGAACATCTGCTCTGAGGC
AAACACATCTCAGGGCAGTGATTCATATGCAGCACTCACTGAGGCGGCTG
TGATATTCTGGTGGCCAAGTTATTCATTAAAGTGATTTGTGGGAAGTAAT
GGATATTCAACTCAAATGGTTGCCAGGGAACATGAGCTTCTAAATGTAGG
AGCTGTCATTGATGGGAGGAACTTCAGGAGCATCTTAGGTTTGCCAGGGA
AGGCCTCCCAGAGCTTTCTGCTGTGGCCTGGGGCAAAAACTGAGGGCTGT
GAACCTCAGTCCCTGAGAACCCCTGGGCCAGGAGGCAGGAGGGTGCTGAG
GACGGAGCCTTCTCAGCAAGGAGAGACCACCACATATTGGAATTTGGAGA
GTCTGTAGGAGTGCTCACAGCACACAGGCTTGCTGTGCTAGGCTGAGTTC
CATTTCTGTCTTTTTGCCCCCAGATAGTTTGGAAATGGCAAACGTTTTGG
GAGGCTGGAGTCAGGAGTGTTCTCACTAATGATGTTTACAGGTTCAAGTT
AAATACTGTAGGTAAGCAAGGAATTTGAGAAACTGGCTGGTGGGGGTCCC
CTTTGTGGCTACTCTTGCCCTTATTTTCCATGCCTTAACTAAACATTTCA
TGGTGCAGAGAAGACCTGGATGTCCTGAGTGACCAATCCCTTTCACGAGG
GTGGCGATCGCTGTGGTCTACTCGCCTTGTTCCCTGGGGTCATGGCGGTG
GGGTGGAAGAGAGGAAGTTCTTGTTGCTGTCATCTAAACACGGTTTTCTG
ATAGGAGAGGAGCAAGCATGCCTCTCTTGATCTCCTGCCTTGACCTCTGC
ATGTAACAGTTTCCTTTCCTCTCTCACATCAGCCTGGAACCTGGGGGAGC
TTTTCTGAGAAGGGATTTGGGTATTTTCTGTAGCATTGACTGAAGCCCCT
CACTCTCACCTTTCATTCCGTTTCTCCCACCTTTCCTTACCCAGACCCTG
CAGATGTTTCTCACCCTCTTTCCCTGTCTTTGCTGATCTGAGGAGTAGAG
AGAGGGGGAAGGCAAACAGAGGGGAAAGGGAGGAGAAAATGCCTTGGGAG
ACACAGGGAAAAGTCAAAGAGGTGAGGTCACAGAAGGAGCAGCCAGCCCT
CTGTTGAGAAAAGTCCAGTCCGGTGCAGCCTCCAGTGATAAAGCAGACTC
CTCCCATCTAAAGACTAGACAAGGCTGGGCGTGGTGGCTCACATCTGTAA
TCCCAGCACTTTGGGAGGCTGACGGGGGTGGATTACCTGAGGCCAGGAGT
TTGAGACCAGCCTGGCCAACATGGTGAGACCCCACCTCTACTGAAAATAC
AAAAATTAGTTGGGCACGGTGGCGTGGGTCTGTAGTCCCAGCTACTTGGG
ATGCTGAGGCAGGAGAATCCCTTGAATGCAGAGGTTGCAGTGAGTTGAGA
TCGTGCCATTGCACTCCAGCCTGGAAGACAGAGTGAGACTCTGTCTCAGA
AAAAAAAAAAAAGACCAAAGACTCTGTGTGGACCCACCCACCAACAGCCC
CCATGGGGCTCCAGATCCTTGCTTTCTCAGAAGCCAGGGATCAGATGTAT
TAGAACATATGCCAAGTGATGAGGCCTTTGTCCTCCTGCTTTCTTGCTAA
TTTAGCAAGAGAAGTTCTGTTAGGAACAGGTATCCTTGCGGCTGTATTTC
AGGCAAGCCCTCCATCTTAGCAAGGGAAATGTGCAGATGTGCAGAGTTTA
ACAAGGCACAAAGCTCTGGTCTCAGGGAGCAGAAGCAAAAGGTAGCAGAC
ATAATAAGACAAGACATTTACCTTACCTACAGTCTGGCTCAGGGAGCTCC
AACATTGTGTAAAGCCCAAGGGGAGTTTATTTCAAAATCCCAAATGTTGC
GACTGGGTTGCCATTTTAAATTGGGGATTGGTCTTGGATTGATTCCATGT
GCACCAGAAGGGACCTTACCTTTTCTTCCAGGGTGTGAGGTTGAATAGTT
GACAGAAGTGAGGCTGCCTTTTCCTAGAGTTAGTTTCCCTAGAGTTAGAG
TTTCCGTAGGCTAAAACCCAGTGATCTATTCCTAATGCTCCTGCCAGTTA
GCTCCCTGCACCGCCTTCCCCAGGTGTGTGACTCAAGTTGTCACAATCTG
TTTACGAGGCTGGAGAGAACTGGTCTTATTGCCCTAGGTGGTCAGAAGGT
GCCTAGCTTCTCCCTCAACACCTCCCATCGGCCCTTTTCCCTTTGCTGTA
TGCACCCCCATCCCTACCTCCCTGACCCCTCTCCTCAGTGACCTCCTCTC
TGTGGAATTGAAGAGAGGACCACTCAGACCCGTGTCCCTGGCCTGACTGC
ATCTGCATGTGCTCCCACACTCCCAGTGCCTCCCTGGACATCTCTACTTG
GTCTCCCAGCCCCACAGCCTCAAGCTGCCTCCCTGCACTCGTCCGGCTCC
CCCTGGCATCTTGAGCTCCTTGCTTCCCTGATGTCACCCTCCCTTCTCTT
GGACTCAGATATGCACTCGTCATCCCCCACATCTCCTAATCATCGGCCAT
TGCCTCCCCTCAGTGTCTCTCCCTCTGCTGTCCCCTGGCCACTGCCACCG
CCTGACTGGAGGGTTTTGTTTTGTTTTGTTTTGTTTTTGCCTTGTCCCAA
GATCTTGCGTGGCCTCCGTGCCTCTAGCCTGTTACAGATTCCTCTTTCTA
AAGTGTTTGACATGCATTATCTCTGCTCAGAAGTCTTCGGTGATGGCCAG
ATAGAGATAAAATTCCGTAGCTTCTCTCAGGTACCAACCCTTTTTCTGAC
TTGGTTTTCTCTTCTCACCTACCTGGAACTCTCTCAGACTAACTCAATCT
TTTTCAGACCTCTAATCACCCTTACATTTTTCCTCTCCTCATCTTGTCTC
CTAGGACTAGGGATGACCCCTTTTTTCCTACCCACTTTCTGAAATTCTCC
TCATCTTCAAAACCTCAGCTCCAGCCTCACTTTTCCAGGGATCTCTCCTC
ATCTTCCTGTGTTCTGAGCCCCTCTGACACCACTACTTGTACCTCTCCTT
GGCATCTGCCATATCACCCTGAGAGTAGCTGTCACTGTTTTGTGCATGTG
CCTCATCTTAGTAATAGTATGCCCCTGGGGGGCTGGGGGAGGCAGCCCTG
CTCGGCCCTTGGTCTCCCATGAATGTGCTTGGTCTAGAATAATAATCAAT
AAATACTTGCTTACCATATAATTTCAAATGTTCCTTCTGGGCAAAATTAG
CAATAATACACCCACCCATACTCACACACAGCCCCTGATAAAATCTCTCC
AAGCTGGCAGGCCAGGGGAATTGTAAACTGTGTGCATCATTGCCAGAGGA
AAGCACCATTTCAGTCATCACAGGGAGACCCAGCATCCATTTCCTGATAA
GTCTTGAACAGTGTGTGTTTTGAACTGCGATCTTCAGGAGTGTATCTCCA
TGTAAGCCATGCCTGGCCTGGAATCACAGGTGTGATGGTAGAGAGAGGCT
CACAGGTCCCTGGGGGAGCCTGTAAGCCGAGGAGTAACCTAGAGAAGGGA
TCAGGAAAGGCATCCTGGCGAGGCGGTGTTCCAACAGAGCTGCCAATGGA
GTCAGAGCTAGGCAGGCAAGAAGGCCAGAGAGGGGGCAAGAGAGTGCTCT
AGGCAGAGAGTTAGAATACGCAAGGAGTCAGGATCCAGATATGGAGGTAT
GGCAAAACGTCAGTGTGACAGCTGCCACCACCTTCCTGAGCCGGCCATGC
CAGGGGAAGAGGCCTCTTCCAGGGATGTGTGCTGGGAGGCACAGGTTTAG
AATGAGTCTGGAAAAGCAAATCAACATGGAGCTTTGTCCTTTATAGGATC
TGGAAGAATCGGTGAATTCACTGGTCGGTGGAGAAATGTCTGTAGACCCA
CTCTGGTGGTTGCAAGAATTTGCCATCAGCAGAGAAGGCAGGGCGCCATT
CTGCACGACAGCTTCGAGAGGAAGGAGAGGAAAAGGTTTGACTTACGGGG
AAAAGGAAGGCCCCTTCAGTTTGTGTGGGCTTCCTGTAGCTACGTGGCTC
ACACAGAGGACAGGTCTGTGCATTTGAGGAAGATGCTCATGGGACATGAA
CAGTCAGAACCCTCCAGGTGTGGACAAAAGGCCAGTGTGGACAGGAGCGC
CTGGGAATGAGAAAAGAGGATGCCCAGGCAGGGGAGTGGAGCCCAAAGGC
AAGACAGGTCAAGGGGTGGGAGCAGGGGGAGGCAGGACATGGGCGGCAGA
CACAGGAGGCCTGGCTGTCCCCACATCTGTCTCCAGCAAACACTAACTGA
GTAACAGACGAATTAAGGAAATAGTGAGCAAATTTGGAGGGACTTGAAGA
ATTTAAAAATAAAGTGACTGCTTCCTAAGTCACCTGAGAGCCAGAAAAGG
ATAAGCAGACTCTCCCCAGAGCTGGAGGAGGAAATGTGTGGTGGGCCTAG
GAAAGATGAAATGGGAAGCCTGCGCTGGAAGGGACAGGATTCTGCCATGG
AACCAAGGGGATCCCATGGCATGTCTCCTGCTGCAGCTGTGCACCTCCTG
GAGCCTGCCTGTATAGAGCAGGAGGCGGCTCTTGCTGAAGGCAGGTTTTC
TGATGAGCGATCCCTGGCATAAGACAGCCAGCACCTCCCCGGCACCGTGA
GCAAGGCTGTGAGCTAGGGCTTTACAATGTCTCCAAACACTCCTAGACAG
TCACTACTGTTATCACCACCTCACATGTGAGAATACTGAAGCTTAACATA
AATAGTGTAACTTGCCCGTCATTTTACAGTCTGTAATACTGGGCAAAGTC
AAGAATCAAATTCAGGTCTCTCAACAAAGCTCATGCCCTTACCCTTGCCT
TCAGGCCAAACCTCTGGCCCGGGTATTCATTTCTTAGAGCTGCCATTTAA
AAAATACCACAAACTTGGTGGCTTAAAACAACAGAGATGTATTCTCTCAC
AGTTCTGGAGACTCAAAGTGCAAGGCAAGGTGTTGGCAGAGTTGGTTCCT
TCTGAGGGGGAACCCATTCCAGGACTCTCTCCTTGCTTCTAGCAGTTGTT
GGAAACTTGTGGTGTTCCTTGGCTTGCAGACACCCTACTCCAGCTTCTGC
CTCGATTGTCAAATGGTATTCTTCCCTGTGTGTCTTTGTATCCAAATTTC
CTCTTTTTGTAAGAATGCCAGGCAATGGATTAGAGCCCACTTGATTTCAG
CATGGTCTCATGTTAACTTGATTACATCTGTGAAAACCCTATTATTTCCA
AATAATGTCACGGTCATGAGCACCGGGGGTTAAACCCACCACATATCTTT
ATGAGAGGCATGATTTGACCTGTGATACCTAGCAAGGAGACTGGGGGCAG
AGAATGTCCTCAGCATTTTGCTTTGAGTTGCAGAGGCAGAGGGAGTAGGG
TGGCACCAGCCGTGGGGATCATAGCAAAGAGTGCTGGGGGCTGCTTTTGG
GGCTGGAGGTGAGAGGCACTCTGGGGAGGGAAGTGGAGATCCCCAGGGTG
TGTTGAGGATGGACATATCCAGGAGATGCGGCCCTTCAGGTCTGTGTCTT
TGGTACTTTGTTCTTGATCATTGGTAAGCCAAGACAGTCGGTTATGTGAC
CCTCTTATGATCCACAGGAGGTCAAGTGGAATCTGGAATTGTGCTCAGAG
CTTAGCACTTAGGGAGGACTTTCTCTGGAAGTATCCCATAGGACAGAGAG
ATTTGAATGTTGGACAAAGCCTTCTCCTACTGCCATCCTTCTACCTGCCA
CCAGAAAAACTGGGACGAGTGACCACAGAAGTGTGCTTTTTTCCCATGTG
GAGGGAACCTACTGTGTTGCACACTGGGTTTTTATTTTGTTTATTTGACC
ACTTTATCCTGGCTGCCAAAGACTTGCTGTATATGGGACGCAGTTTTCAG
GGGGCTGCTCATCACTTTTGGAATGATGAGGTCTTGGAAGACAGCCTGTG
GTCATGAATCCCAGATCTCTCGGCAGGCAGGGGAATGCCAGGGTGACCAG
ACTGGGGTGTGGAAATGTTCCCATGCCAGAAGGTGTGTCTGTCAGCAGCA
GGCTGTTGCAGGGGAGCCGCCGCCTCTGCCAGTGTCGTGAGGTTCAGCGG
GAGGCTAAGCTAGCCTGTCTGTAGCATGTGTGACTCCGGAATGCCGTAGC
CTCAGGAGCAACTCCACTGTCAGAGCAGAGCACTGGGGAGAGCCGACAGG
CGTCCATGCACGTCCGTCCTACCTTCTGCTCTCATGGGGTGGGCAGCCTG
CCCGCCCTGCTTCTAAGCATCTGTTGTTTTGGGTCTGATTACAGACTCTT
GAAGCTTTGTTCTTAGACCCCCACTGTGGCTTTGAAAAGGCCGAGTGTGT
TGTGGTTTGTTGGGAGGTCTTCTCAGAGTGGCTGCTGCCAAAGGTGGTGG
GTGGGCCTAGGCCACCCCCAGGCAGAGGTTCCCCCAGATGAGGGCTCCTT
CCCCATCCTGCCAGGGCACTAACTTCTTCTTGATGAAACCTCCTCGGGCT
GTTCACCACCAGCCCCAAGGACGCCCTCTCCTCCTCACTGCAAAGAGGAG
TGAGTCTCCAGTCTCTTGCCTGCTACAGAAAAACCTTGAAAAGGCCAAGA
GTCAAAACTGGCTCCAATTCTCCAAATGGAGATGGTTGTATTGGGAGCTC
TGGTATGGAGCTGGCCCTAAAATGTCGACAAGGGCTTGCTGAATAACTTA
CTATTCCTGCAGAAATTTGGGGGGAGATAAATGGATACAATAGAAGGAAA
ATACCTGTTTCTACAATGAATTTATAACCCAATTTGTTAATTTTATATTC
ACTCACTTATGGCGTATGAGTGCCTAGGTAACCAGTGGGGTCATTCGCTG
ACATTTATTCTTTTCTTCTGAAATCATTCGTCATTGTTTGTGGAGAAGAT
TACTCCCTGCTTCTTTGGTCATTTCCCTCCTCTGCCTTGCTGGCACCACA
GGTGTGTCCTGGGTGACAGAATTGATGTGGGGCCCTAAACACTCATGGTG
GTTGTAATTGCAATCTGGGCTCAGTGCCCTGAAGGAACAATAGAGACCAG
GTAACAGGAGGGACATTCTTTACTACCAGGAAGATGGATAAGTAGAAGCT
CACCCCAGGCAGTGGGGATGGCCCCCACCCACAAAGACTCTGAGACAAAA
CATCACCTGAGGAGGGCCATGCATTTTCCACTCAGCTACAGATTTCTACT
GCTTATTTTCAAGCAATGATGTAAGTAGCTTGGTACGGAGAGGCAGCCAC
GGTGGTGGGGAGCTCAGTGACAGGGGGAGGAGTAGGGGTGCTTTGCCTGA
ATTTTGATCTGAGGTGGGGGGAATCCATTGGAGGGTTTTTACAGGGCTGT
GACAGGCTCTGATCTCCATTTTTGAAGATCATGCTGACACACTATATGAT
TCTATTTATATAAAGTACAAAACAGGCAAACTCATCTTTGCTGTTAGAAG
TCAAAAGACTGGCTCCCCTGGAGGTGTGTGGTGACTGAGAGGGGCTCTAG
GGGTTTCTGGGAAGCTGATGATGATCTCTTTCTTCATCTGGGTACCAGTC
CCTCGGGTGCACCTAGCTTGTGAAATGTTTCAAGCTGTACTCTTATGATT
TATGCATTTTTAAAAGATGAGTGTTATATATCAGTAAAGTTCTTGAAAGA
TTACTCTGGCAGTTGGTTAAAGAATAGATCAGAAGAAAAGAGTAGGAAAC
GGGTAGGAAGTCATGATAGTTGGCCAGATGAGAGCTGACTGGGACCTGGA
CTGGAGGTGGCCAAGCAGGTGGAGCGTGCAGAGTGGACTTGGAGCTTGGT
TTGGAGTTGGAAGAAGCGGGGCAAGAACTGGAATGGGGAGGAGACAGTGT
GAGGTCCCAACAATGGTTGGGGGCATGGATTTGAGGAACTGAGGGGAAGT
CAGAGTTGAAATGTGTAAGATGCAGTTTGATACGAGGCCTGGCAGACTTC
AGTGTGAAGCCCAGCTTCCCCCCAAGGGTGCATGAGCAGGGCACCCATGG
GAGACATGGCGGAAGATGGGCACAGGACTAATGACAGAACCCAGACACCA
TCAACATCATGTGGGGAGAAGGAAGAGGGCTGCCACAAGGATTTCAAAGA
AGGAACAGTCAGAGAGAGAGAGCTAGAACACAGGGACAGCAAGTGACATC
ACAAATGGCAAGGGGAACCCTGCAGAAGGTAGGTAAAGCCATTGTTGGAG
GGCCCACTGGACATGGCACTGAGGTAACTGGCAGTCTTGGTAAAAACAAT
TCGGTAGCACATTCCCGCTCACCCCAGGCAGCATGGTGGAGCAAGGTGAT
GGATTTGGCATCAGTAGCCTGCTACTATTTGCCAACTCTGCCCTGAGCCT
CAGTGCCTCAACTGTAAACAAAAGGGATCACATGTACCTCTCAGGGCTGA
TATGAGGATTAACTTTATTCAGCGAATATTTATTAAACATTTATTATATC
TCAGATAGTCCGCCAGACACAGAAAATACAGAGTGACCAAGAGTGACAAG
TGGGGAGACAGAAATAAGTAAATCAATGAGTTGATGGGAAGAAAATAAAT
GCCCCCCATGCTTCCCTGCAGCCACCACCCAGCCTTCTTGGACTGCAGGG
TCCCTCCTTTCCTGACTGGATTCCTTGGGGTCAGCTCCTAAGATCCTAGC
TCTTCTGATACCATGTCCCCAAGCCCCACTTTCTCACTCTGAAAAACTAG
CTTCTCTTTCCTTGAAGGCAGGAAGAGAAGGGCTTAATCTTCAAACTCTT
CCAGGATGCAAGAAAAAATATTGACATCCCATCCTATTATTAGAACAAAG
AGTAGGAAGAATATAAAATAGTGAAGTTTCCATATTGATGTTTGAATAAT
ATCTAATAGGTCTGAATGTCCTATACCTGCCATAAATGGATGCAAAGCAG
GTTCTTTTCAACCTAACAAATTACTGCCAAGTTCCCAGAAAGACAAGATG
GCACTTGATGAAAATGTTTCTGGCTTAAAAAAAAAATTAATTGGAGAATT
TCTAAAGAAGAAATTGGACACATATTTACATAAATATCCGTGATAATTAT
GTTTTCAAGTGTTTTTTTTTCTGGGAAACCAGGCTTCCAGCTGACCTGGA
AAATTCATCTTGTCTCACATGTTTATGTTTCATCTCTTCCAGAATTTTTT
TTCAATCTGTGGTATCTAAAGCCCAGACCCTTGGCCCCCTGCCATGGGGC
AATCTTTCACTTGTGAGCTTCCCTTTCACCTTCTTTCACAGACCTCACCA
CTCCAGCTATCCACATGCTCCATTCCTACTCCTGAGCTTTGAACAGGGCA
CCATTAGAAAAGAAACACAACACAAAACAACGGAAGCAGGATATTTCCCT
TACCCCTTCACGGGCGGGAACTGGAGTGCACGGGAACTGGACCTAGCTGG
TCGCTTCGGCACTGGCAAGGATGAATTCCACTCACTCAAGTCCACTGTGT
TCTACTCCCTGGGGAGGGGGAGCACAGGTCAGCGGGTGCAAGAGCTCTGG
GGCTAGTACTTTTGGACTCCAGCAGGAGCAAAACTCCTTGTGGGCCCCAC
GGCAGCATCTGGAGGTGGGGTGCCCATGACCCCTGAAGCCCCAGAAGGAA
TGTTACAGTGCCCTTTTAGCCCATCTGTGGACGGCTTAAGTGTTAACAGC
TCAGTGCAGGGTCAGTGTGACAGCACTCATGACAGCACCCACACTCATGA
CAGCACCCACACTCATGACATCTGAACTCTTGTCTGGCGTCCAAGAGTAA
TAAGGTTGCACGAACGGATTGAAGATGGCAAATGCAGGGGATTTAACAGC
CAATGAAAGTGGCTCAGCTGGAAGGGGAGCTGAAAAGGGAACAGGAAGGT
AATATTCCCTGGAGTCCAGCTGTCCCTGGACAGACACCTCTCTGAAGCTA
CACTGTCAAACTGTTCCTCTGAAGTCAAGATGCTTCTCTCCAACATCCAA
CTGTAGTCTCTGATGTCCAGCTGTTTCTCCTCCCTCTGCTGGCAGAGCCT
GGGGTTTTTATGGGCTCAGGATGGGGGTGCAGGGCGGGCCATGGGTGGTT
ATGGAAAAGGCAACATTCAAGCCGGAAAACAGGAATGTAAGTTCTCACTT
TGGGCCACAGTTCCAGGCTTGAGGGTGGGGCCCTCGCCAGGGACCTGCCC
TCTTCTGCCTGCCTGGAATTTTTCTGCCTCCTGTCCCTATCACAGCCACA
ATACAAAGCTTTTTAGGATGACTGGTGTTATTAGGAGCTAAGAATTTTTT
CTACCTCCCTTTCTTGTGCTCTATCTTCCTTCAAACAATGCCTCTGCAGT
AGAAATCTGTTTCTTTTTCAGATTGTCCTGACCTTTGTCCTTTCAAGGGC
CCCCAGGAGCCTGTCTTATCAAAGGCTTTTTTAAGCTTATAATAACACAA
GGTGCAAAAATACAAAATAACAACAAACACTACAGAATTATGCAGTTTGG
GATTTCCAGTGTCCATTTAAATTGTATCTTATATGATGACCTTCAAGTGA
TTTTTGCTGAAGTTGAGCGTACTAAACTAAAAAAGTGGTCAAGAGATCCT
GGGCAAGTCCGATTCGGTCAGGACTTTGCTCAGAGAGTTCCCCTAACCAG
GGTTCCCCAGGCAGTGTTAAAGGTGGAAAGAGAATCCACCTTGCAGTTTG
CTTCTCTGTGCCTGTTGTGGTTTCCTGTGGCCTCCACTGAGGACAGATGT
GCCAGTCCTCCCAGAAGCTGTTCTCATAGGCCTCAGAGGGTGCCTTTGCT
CAGTCATATTGGGGAACTCTGCCAAACAGTAAGGTCAACTTTGTGGGCAG
TAAAGGCTGGCAAAGGGAATCTGGAGAGAAGAAAGCGGAAGAGTTACACT
GAGCAGTGAGAGATCTGTTGGAGGCCTGGCTCCTCCGGCCCCCTGCAGCC
TCCCCTCCCAGGCAGGGTCTCAGGCCCAGCTCCACAGCCTAGCCACTCCC
CTCTTAAGGAACAAGGAAGCTGTGATGGAAATCTATGTGTTTCTTCCTAG
ATTGTTCTGATTTTTATATCTCCAGTGACCCACAGGACCATGTCTTATCA
AAGGCTTTTTCTAATTCATAATAAAATGAATAATACTAAAGATACCTTTT
GTCATTGCTATGAGGAATATCACCAACTTTGACCCCAGGCTGGCTATTTT
AAATCTCTGAACTTTATCTTGTGCAGGCATTCTTCCTGCGTGGGAAAATA
TTGGATTTCCTGAATCACTTCTTGAAAGAGTCATTCCCCTTTGGGGCATT
GGGCCATGGTTTAGATTCACAGCCACACCCTGCAGAAGGGACAGGAGGGG
TGAGCAGGCCAACGGCTGATGGAGGGCTTTTATTCTAGAATGCACTGGAC
TCTTGCTTTGCTTCCTTCATTTTAGGGGTTGGGGACAGGGAGAAGGATGC
TACTCCCTGCTTCAGCTCCGAGGATCTTTCAGATGCACCTCAGAGGAGCC
AACCTAGAAGCCTGCCCACCTTCAGATGCCTGCAGTTTTGTTTTTTGCTT
GTTTTTTTTTGTTTTGTTTTTTTGAGATGGAGTCTTGCTCTGTCACCCAG
GCTGGAGTGCTGTGGTGCGATCTTGGCTCACTGCAGTCTCTGCCTCCCAG
GTTTAAGCGATTCTCCTGCCTCAGCTTCCTGAGTAGCTGGTATTACAGGC
CTTTGCCCCCACACCTGGCTAATTTTTGTATTTTTAGTAGAGATGGGGTT
TTACCATGTTGGCGAGGCTGGTCTTGAACTCCCGACCTCAGGTGATCTGC
CCGCTTCAGCCTCTCAAAGTGCTGGGATTACAGCAGTGAGCCACTATGCT
TGGCCAGATGCCTGCATGTCTGATGACACTGGGAAGCAGCCAGGAGTCAC
CCTTCTGAGTTCACTTCTTTATCCTTATTGGCCTCTTAAGCCCCAAGCCT
CACCTCCCAGGTGAGGAGAACTGCCAGCCAGGACCAAGAATGAGTTGTGT
GAAGGCAGACAGTGCGATGGAGAAGTGGAGATGGGCATACACCCAGAGCC
AGCACCACTCTCAGGCCACCTGCCATCTGTTGGAGACACACCAGGCATTT
TGGAAGACTTAGTCAAGTAAAGGTGGTATCGGGACTCATGAGTGTAGTGG
GAGGAGAGGAAGGGTTCGGTATATTTTTTGGTTTGACAGCCTAGTCCACT
GACAAGTACATATTTCTTGAAGCCTTTCTTTTTCCCATTGAAAAACGGGA
CAGAATGATTTGTCTGCTAGGTTTAGCACCAGGAAATAGATTGGTTTATG
ATTCAAGATTGCTGACTGAACACATTTGTCTCCCCTCCTCTCTGACACTT
TCATTATAATGAAACTAATGAAGAAAAAGGAGAAAAATCAATGACAAAAA
ACTGAAGAAGGAGGGAGACTATGAGTGGATGAAAGATTTCAATAATTTCA
GCAGGGTGGACAGTGGTTGGGAAGTGTTGATGGATAAAATAGAATGGAGG
AAGACAGATGGTCAACTGCGTGCACGGGGCCTGCAGTAGCGGGGAGCCCG
CGGGCTTGGCACAGCCCGGAGAGGCAACAGATTTAGAATTGGCGAACGTG
GCAGAAGGAGAAGTGAGATTAAGGGATTAAGATAGAACAGCCCTTTACCC
CACGGAAAACACAAGAAACCTTCTCTCCTAGAATTAACGGGAACTATCAC
ACAGTGTGAAGGTTTGTGCCCAGGAGGGAAGCCCTGCTCCTCTGGCTCCC
AGTTTGCTTCCCACAAAGTGATCTGATAATAAAAATACTGATAGAACTAT
TAAAAGTTGGGAGGAGTAGGGAGAAGGGGGCTTTGCAATTATAACTGTGC
TAAATCCTCTTCTGTAGTAACAGGCACCATAAAATGGTAAGTCAAGAAAC
TGCAGAACAAGTGTATTATTTTGAGATGTGTGGATGTCATCAACAGAAAA
ACTAAAAACTAAAAGCAGAGGGGGTTATTCCCAACCTGCGGTACTGGTGG
TGGGCTGACTTGAGTGTAGAGTTGGGCTTTGTCATTCTAAGCCCTTCCAC
ACTACTTGATATTTTAAACACATGCACATGTTACACTGATTCAAAAAAAT
AAAATAAAATGTGGAAAGAATTGCAAATTGATTTTAATTCCATAGGTGGG
TTCCAGTTAACTGTGGAATGGGAGGACTGGATGCCTGTCCAGTGTTGCTC
AAATCCAGGGGGGTTGTCAGTGTGGTTGTGCAGTTATGCTTCTTCCCAAG
AGAGGGAAGTCTCAGAGGGGTGTTACCACTGGAGGGTCTTGACTATGAGT
TGTCCAGGTCCTTGGCATTTTGAACAAAGAACTGAACAAAATGCAGAAAC
AAAGTACCAAAGGAATGAAACACAGGAATGAAGCAGTGAAAACAGGAATT
TATTAAAGCAAGAAAGAACTCCGCAGGGTGGGAGTGGGCCCTAGCAAGCA
GCTCAAAGGCCCAGTTATAAAGTTTTCTGGGCTGTAAGTAACCATTTGAG
GTTCTTCCCTTTATCTGGATGAAGGATTTGGCTTGTGGCTAATTAAAGGC
TGAGGTGAATTGGCGCCATATGCAGATAAAGGGAAGGTCCCTGCAGGGCC
CGCCGGCCAATCTAAGGCACTCTCCTTTTCCATCTGAGACGTGGTGGAAG
GGGGAGGGTTGCAAGGAGAGTAGCCTTTGATCCTTTGTTACTCAGAGTGG
GAAGATGGGGTTTTCCCTTTTGCTTTAGCTTTAGGAAGTTTGTGTTAATT
GGCATTAGATTCCCTGCCCCCAGACCTTGATGTTTTCTCTTTTAGGAAGT
TAGCACAAACCGGCCTTAAGTTCCTTGTCTCCAGACTCTGTTCTCCTGTC
TCACGTGTTCCCACCTGTCTGGTATGCAGTCAGCTCGGCCTAGAGAGCAG
AAGAGCAGAATGTCCCCTGAGGCCAAATTGACTTTCAGAGGTTGGGAGGG
ACTAAAGCCAGCAGCACATGGCTGTCCCCTCCCTCTTTCAGAGAACAGCA
GCATGGTCCTCAGTACCCCGAACTCTCCCTCCCTGCATCCTGTTGTCGAC
AAAAAGAGTCAAACTCTGGCTCCGAATACATCTCTTCAAATATTTTACAA
ATGTGAGTGACCATGGCCTGTGACACAGCCCTCAGTAAGTCCTGAGAACA
TGTGCCATGGTGGTCGGGGTGCAGCTGGTTTTATACATTTTAGGGAGACA
TGAGACTTCGATCAAATACATTCAAGAAATACATTGGTTTGGTCCAGAAA
GGCGGCACAACTTGCAGTGGGGGCTTCCAGCCTATAGTTTAGGTTCAAAA
ATTTTCTGATTGACAGTTGGTTGGGTTTATCTAAAGACCTGGGATCAATA
GAAAGGAAAGTCTGGGTAAAGTTAAAATAAAGGATTATGGAGACCCAAGT
TCTTATTTGCAGAGGAAGCCTTCAAGTAGTAGGCTTCAGAGGGAATAGGT
TGTAAAATGTTTCTTATCAGGCTTACAGACTGTGTCAATGTTAATGCTGG
AGAGGTATAATGAGGCATGTCACCTTATTATACTCCCACTTCCTGTCATG
GCCTGAAACAGTCTCTCAGGTTAAATTTTAAGAGTGCCCTGACCAGGAGG
AAGTCCATTCAGATGCTTGGGGCAGTGAGGGGGAATACTTAGAATTTTCT
TTTTGGTTTACACTCAGTGAGGGTGAGAATAAGGTTTTTCTGTGCTGTTC
TTTTCTTCTCCCTTTTCCTCCCTCCTTTCTTCCTTCCTTTAGAAGAACTG
TTTCATTCAGATTGCCCTGTCAGAGTCCTGTTTGGGAAAGCAGATAGCTG
CCATTTAGGGATAGAATCAGTTATTTCAAACTGAGAGGTCACCGGCTGTG
TGTCTCAGTCCAGCCACATCTCCTGCCCTGAATGGTTTTCCTCCAACTCC
TGGACTCTTCCAGTACATTGAATCACCTCAGAGAAGTTGCATCCTAGTGG
ATATCGATGAAAAGAGTCAAACTCTAAGATATTTGAAGAGATTTATTCTG
AGCCAAATATGAGTGACCATGGCCCGTGACAGGGCCTGAGAACATGTTCC
CAAGGTGGTCAGGGCGCAGCTTGGTTTTGTACATTTTAGGGAGACAGGAG
ACTTCAATCAAATACATTTAAGAAATACATTGGTTTGGTCCAGAAAGGCG
AGACAACTTGAAGTGGGGTGGGGGGAGGACTTCCAGTTTATAGGTAGATT
TAAAATTTTCTGATTGACATTGGTTGAGTTTATCTAAAGACCTGGGAACA
ATAGAAAGGAAATGTCTGGGTTGCGATAAGAGGTTGTGGAGACCAGGGTT
TTATCAGACAGATGAAGCCTCCAGGTAGCCGGCTGGCTTCAGAGAGAATC
GATTCTACATGTTTCTTATCAGACTTCAGGTCTGTGTTGATGTTAATGCC
AGAGAGGTATAATGAGGTCTGTCTGACCCCCACTTCCTGTCGAGACCTGA
ACCAGTCTTTCAGGTTAAATTTTAAGAGTACTCTGGCTGAGGAGGAAGTC
CATTCACAATGGGGATGGGTGGGGGTGGCGGGGCGGGGGCGGGGGGGGGG
GCTTTGAATTTTATTTTTGGTTTACATGGACATAAGACATCAGTTGAAAT
TTTCTAGTCTTCCTTCCCTGGGGTTTGTGTGGAAAACATCAGAGTGGGTT
GACAGCACAGTCAGTCATTAAATAGGTAATAACCCTATGGGGATCACATT
TCAGACCTTCCGCTGGTGTTCCTATAACCACGGTTAGATGCCACTGTGAC
CTAACAATGGTTAGATCGTAGTTCCGATGTTTAGTTTGGAAAAGAGAGGC
ATGAACATGTAGATCAAAAAAAGCCATGTCTAGACTGTTCTTTGATGCTT
TCTCCTTCATCCTGAGCTGTGGGAGCCTCCCTCCTTGTCCTGTGGCTTAT
CCACCATGGTCATCTCACTTATGTCACTGAAGCGTGGCTTGGGCCAAGTC
CCCCACACCCCCAAATTTACCTTGCATCTCAGTAGCTTTTTAGCCTTTCT
TAGCTCTTTGACACGGGTGGTAAAAAGTTCTCTCTTTATTAAAGAACTTC
GTGGCCACTCCAGTTTCCCCGCTTCCTTCCTCTTTATATGTTTGTTAGGA
TTGGAGAATGAATGAGGCCGTCATGAGCATGGCCTTGACAGTTAGAAGCC
TGAGTGCCTCCTGCCCTCCCTGCCCTGCAAGAATGTTGCTCGTCCTCTGT
GGGGTTTCTCTGCCAGCTTCCTTCTATAAGATGTGTCTCAAGTGATGTGT
TTACCTTAACACACACGCACAGCCCTTGTCAACCTAGATGTTTAGATTAG
CACACACACACAAACCTAGATATTTGCCTGGGTTTTGAACCTGTCAAAAT
AGGACTCTTGAATAAACAAAGTTATTCCAATGACATTGCCAACGCTCATG
ATTGAAAAAACAAAAAACAAAAAACAAAAAACTGTTTGTCAAATTCCCTT
CAAAGCCCCAGAACATTATTTTGAATACCTCATGGTTGCAAATCTTTTTT
CAAATGAATTTCTTCTTTAGCCAACTTTATTGAGGTATCTAGTTTACATA
TAATAAAATGCACACACTTTAAATATTTGTTGAGTTTTGACAAATGTATA
AACCTGTATAAATACCACCACAAAATTAAAAAGTAGAATATTTCCACCAC
GGTAGAAAGTCCCTTCATACATGGCTGCAAATCTTATTTCAGTTTGGGTT
TAGCCAAGTAAGTAAGTAGGGCAGCTGAACTGACAAATGCTGTTTTGTGC
CTTGTAAGTTGTGTGGCTTGTAGATTGATCAGAAGCAGCACCAAAGGGGC
AAATGTATAAATGATTGCAGACAGACCACTGGAACAAGTCTGCAGCCTCC
CAGAATGACTACTGTGGTGGATGTATATTCTGGAGCTTGGACTTATCAGT
TAGAGTACTTTATAGACATATTGTTCATTTGACAAAGATTGAGCCCCAAT
TTTGAACTGGGCATCATTTCATGCACTGTGGATTTGTTCTAAAAAACACA
CATTGTATTTCCACCATGAAGTCTATATTCTGGTGGGAGGAAATGTCAAT
AAATGAGAAATCAAACGCATGATGTAATTTGGGGAAGCAATACATGCCGA
GAAAAGAAAAAAAGTCGAGTGAGGAGATAGAAAATGATGGTATATACCAT
TTTAAAGAAGATAATGCAGCCCGTGTAATTCATCTTCATTTTCTTTTCCT
TTTGTGAAGAAATTGCAAGACTCAAATTCTACCAAAGGTGTTTTATAAAA
GGCACATGTTATCTTTCCATAAAGTGTATTCTGGAGAGTGCTAGCATCTA
TTATAAAGTAGTGGTGATGTGTGTGTGGATGCGGAGTTACATAGTTAAAG
AAGCATGGGAAGGTTGACTTAAACAACTTAACTAAGTCTTTACTACGGAA
CTCAGAACCTTTAATATGCAAATGTGATTGCACATATTCGTGAGGGAGGA
TTGTAGTACGTTATGTTTTCCAATTTGTTTTCTCCAGGGAGTTGTTTGGC
CTAGAGCATCTTAAGCAAGTGATGTTCTGGTAACACATTGTGGGAAATTC
TATTCTAGGGTTCTCAGACTACCGGAGAACTGAAAGCAACCGGGAAGTTA
AATTACTCAGAGGGCAAGAGGAGGGAGTGGAGGTAGCTTCAAGTTACTGA
ATATCCCTCCAGGATGTGGAGCACTCACCAGGCAAGGTGTGGGACGGGCA
GGTGGCAGGGGCCTGCAACATTCTGTGGTGCAAGCCTCACAACTCAGGTG
TTATCATAGTTTTCACTTGCTAATGGGAAAACCTGCTGCTCAAGGAGACC
CAGTTGGTAGCCAAAGGTCACACCATGGCTTACAAAGTCAAGACCTGAAC
CTAGGCCTGTTTGACTCCAAAACTCAAATCATCAAAAACTATAACACTAT
ATTGCCTAAATAATGGGGAAGGGTAGGTTGCAACCCAACTAGCAAACCTT
TTCCTCCCACCAATACATCTCCATGTACTCTTGAACCTGTGTCCTCCTCA
AGGGATGGGCTTGTGTTTTATCTGCACGGTGTCAAGTGTTGCTTGCTCTG
TCACATGCTGGCCATATAGGAGGTACTTGGTAAGTTCGAGCAGCCGAGGT
CATGGTCCCATCACATAAGCTTAGTATGTGGGGCCAGATGGCTGTGACCC
TGAGTGGCCCTGACAGAACCATCCATCGACTTCCTTCCCTTTTCCCTGCC
TTTTCTCCCTGCCTTTTCTACCTTGCAGCCATCAATCTGATCCCTCTCCA
TATCCACTGGAACCAAGGGGTCCAGATATATACAAACACCTCTGCCAGGG
CCCCCATGAGGCACCCAGACCTGACAGGGCTTGCAGGGGCCAATAGCTGG
TGGCGAGATTGGCTCTGTGAAGGGGTGGGGGATGTGGAAAGGAGCCAGCT
CTGGCTAGAGTCTTTCACCCAGTAGCACGGGGGAGAAAGTGGGGGAATTT
CTGTACTTAGAGGCATGTCTTTCAGGAAAAGATCTGAGACAAATTTCCTC
TCCCACCATCCCCATCTTCCTCTCATGAGACAACTGGAGAAGTTGGTGAT
GATTAGTCACTCCTGTTAATTCATTACTAATTTGGATGGAGGGAGACTGG
TTTTTCTAAGCCTCAAGCTCAGAAACTCAAGTGCTCCATAACTGAACTCT
CAGTCCTTGATCTCCTTATGCTCTCCTCCTTCTGGATCCCCCGTTTCCAT
GCATGCATGCCTTTGACCTCTTCCTGGCCCTCAGGCTGGAAACAGCCCCG
TCTCTTCTTTCACGTTGAATCGGTGACAGCGGTGTCAAGCCAGCCTTTGC
AAGAGCCTCTAATTTGGTGCCTCTCCAGACCCACTACCCCAGCCAGCCCT
CGTCTCTCCCAAAGCTGTATGACAGCCCTCATTCCTCTGCTAGATGCTAC
TCTTGCCACCAGCCATGTGCCAATCCTTCAGATGCTTCTGTACAGGAGTC
AGCACAGGGTGCTGGGATTTTTAGAAGCAACCTTGAACTAGACACACAAC
TGATCTCCATGCTGGGCCCCACACTGCTCCTGAAGGGCAGGGGAAGTTAT
CCAGACAAAGAAGGGGAAGACTGTGAGCAAAGGCACTGACATAGGGCCTT
CAGGGAACTCTGAGAGTGTGACCTTGATGTGTTTGAAGGACAAGGTGGCA
GCAGGTGCTGCAGCTGTCTAATGCTGCATACCAGAACAATCCAAAACTTG
GTGCTTAAAACAATGGCCACATTTAGGTTGCTGACAAATCTGACAAATCT
TCAATCTGCACAGGGCTCATTGGGGTAGCTCATTTCTGCTTCACTTGGCA
TCGAGGGCTGGGACTGGAATCATCTGAAAGCTGGCTCAGTAACATGTCTG
GTGGTTGATGCTGGCTGAGGACCAAAATCTGAACCGAGTTATCCGTGGAA
CATTAACATGTGGCCTCGCCATGAGTAAGGAAGACTGGGCTTCCTTATGA
TGTAATGTCGGGGTACTAAGGGTCTCAAGGGAGAAAGAGCCAGCCAGATT
CTTTGTCCACTTTTAGGATCCAGCCTCGGAAGTCACATAGCGTCACTTCT
CTTCATTAGAAGCAAGGAGCTAAGATTGGCCCATATTCAGAGGGAGAGAA
CACAGAACCCACCCCTTGATAGAGTGTCATTGTAAGAGGTGTATGTGGAT
GGGAGAGATTGGTGTGGCCATCTTTGCAAAATATGATCAGTCTCAGCAGA
AAATATGGAGGCAGGCTGGGTCCAGGACTGTAGCATCTTGGCTATCACCC
CTCACAAACCAACACAGCTGTTTGGCTAACAGCAGTGTTGGTTTTTACCT
TATAGAAAATGGGACGCCAGTGAGAGAGGCCAATTCAGGCGTGACCTGCT
CGTGTTTGTGCTTGAGAAAGCTCCCCCTGGGGTGGTGTGGTGGAAGATTT
GAGAGGAGCAAGCAGGGGACCTGTGAGAGGCTGTTGCAGTGATCTGAGGA
GGGATGGTGAGGGCCTGAGCCAAGGCTTACACCAGTAGAAGAGGCAGGGC
TCCAATGTGGGAGACACCAAGAAGAGACCATGGGGCTGAGGGATGGGGCA
AACACCAGAGGGTGAGGAGGAGGGACATCAAGAAGGATTATTGGTTTCTG
GCTTGGGAAACTGAGTGAAGGTGATGGGGAATGAAGGAAAAATGGGAGCA
GTTTATGGGGGAAGATGATGAGTCCTATTTGGGAAGTGATTAGTTTGAGG
GGCCTAGGAGTGTCCAGGTGGATACGTCTAGCAGACAGTGGAGTGGACCT
GCTTGCAAAACTCTGGCGAAAGTTCTGGGTACAGAACTGAGATTGGAGGC
CATCATCCTATGGCAAATGGGATTGCCTATGAGCAGGTGCAGAATGGATC
ATGTCCAGAGAAGGGGTCTACAGGCAGGCTTTGGGGGCATTAGTCTTTAG
GGAGTGGCTAGAGGAGGAAGATCTGGCAAGGCATCTGAAGTCAAATTGTC
TCTTTCCCAGGTCTGGCCTGGACCAGGGAACAATCTGGGTTCAAGTGAGC
TTTGGTGTCCTGTTTCCATGGAAAATGACAGAACGTTGACAAATCCCATC
CCCATCACTGTCACAGTCTTGTGCTGCTTTCTTATAACCTATAACCTATA
ATGTGGCTCTTGTCTTTCCTTCAGCCTTATAGTGGCAATTCCTATGGATT
AGGGAATTTCTATGACTGTTTTTCTTTTTCTTGTTTTGTTTTTTTTTTTT
TTTTTTTTGAGACGGAGTCTCGCTCTGTCGCCCACGATGGAATGCAGTGG
CACAATCTCAGCTCACTGCAACCTCCACCTCCCAGGTTCAAGCGATTCTC
CTGCTTCAGCCTCTTGAGTAGCTGGGATTACAGGCATGTGCCACCACGCC
TGGTTAATTTTTGTATTTTTAGTAGAGACGGGGTTTCACCACGTTGGTCA
GGCTGGTCTCGAACTCCTGACCTCATGATCCGCCTGCTTCGAAGTCCCAA
AGTGCTGGGATTACAGGCATGAGCCACCGCGCCCAGCCTTGCTTTTCTTT
TTCTAATCTTTCACATCTTACCCTTTACAAGGATGGTCAGAGCCAGAAAG
AGTCTTCTAGTCCGGTGACTGGAAACCACGTTGTCCCCCAGACTCTGCTG
GTAGAAGTTTTCCAAATAAAGCATTTGAGGCTTCACTCCTGCTGATTTTG
ATTCAGTAGATCTGAGCTTGGGCCTAGCCTCTGTGTTTCAAAGGCACTCC
CACCTCCCCATCCATGTGATTTTGAATCAAAGCTGAGTTATGAACCATTG
ACTTAGTCCAGTTACCTTGCAGTTGGGAAAACCCAGGGCACAGAAGGGGG
AAAAGCACACACAAACCGAGATTGTTCCCTGGTCCAGGCCAGACCTAGGA
ACGAGGCAATTTGACCTCAGATGCCTCGCCAGATTCTCCTCCTCTGACCA
TTCCCTAAAGACTAATGCCCCCTAAACTCTAAACTCTGTCTCTAGACAGA
GACAGGACCCTTTCTGGCTAAGCTTGCTCTGTTGAATAAGAGGCAGGTCC
AGATCTGGTCCATGCCAAGCGTCAGGTGACTAGCATCTAAGCCCCAAAGT
GCCTACTGCATACAGGGCATGGGTTGGGCACTTGGGTTACCCCTTGCTGG
ACTTCAGTTTCCCCATTTGTGAAAAGAGAGTGGGATGACATTATTACAAA
ATATTAAGAAGAATTTCAGAATACAAAAGAAAGCCCAGTGGAGCCTCTCT
AGCATGGCAGGGGTGGAGTGCCACCATGCCCCCAGACAGGCAGGCTTCCT
TCTACCACCTGGGAGGAGTGGGGTGGCCACACAGGTTCAGGTGGCAGTCA
GGAGAGGTGTCTGGTTTTGTGGGTGTAAAGACCTCTCCAGACCTGGAGGC
TGTGAGCATGGACACATGGATAGGCAGTGAGTTCTGGGCTCTGTCTGACA
TCCCAAGCTGGCCTCGCAAAGGTTCTGCCCTGGCAGGAAGGAAACCCCAC
AGCCTGGAACAAGGGCACCTATAGAGGAAGGCTCAGCTTCGGAAGGAAAT
GGAAATGAAGGTGGAGCCCCGGTTGCTCTCCTGTGATGGCAGCTGTTCAT
TCATGGAAAGAGCTTTGGAAGACTGTGAAATGGCACAGCACTCACCCTTC
CCTGGAGGAAAACCTCACCCAGAGGGGCGCCTTCCAGGAGATGCTTGAGA
ATAGGACTCCTGGCTCAGAGCCTGTTCGGTGTCAGCGCAACAGCTGAGAA
CCTGGGGGAGCCCAGAGCTCAGCCTGGAGCCAACTAGGGACATCATGAAA
CCCTTCTCCTACCCTCCCTGATGTGTTATCTGTTCCCATCCACCATGGAA
CTCACATTTACTCAGTACCCCAGGACCAGGTACGAGGCCAGGTGAGATAC
AGAGATAAATGGCCTTGGCCCCTTCCCTGGGGCCTTGATCATCCATTGAG
CAGGCAGCAAGCACAGTCAGTCACAGACATTCCTGAGAGGCCAAGATAGT
CCTGGTAAGAGATGAGGACATGGATGGGAACATTCATTCAAACCAGGTGC
TTTGGGGATTGCATCATGGTGCCTTACTGGAGGCATTAGAGGTGGGCCTC
AGAGGACAGGTGTTGGGAATGGAAGACAGGTCAAGAGAGATGGATCCATG
TGACCCGGGCTTGTTCATCTCCCAAGAGTGGTCTGAATTGGATGGGGAGT
GGCCTTCAGCCCTCACAGGGCACAGGAGGAGGGCAGATGTGCAGCCTGTG
GAGCACTGTGGTGCTGTTGGGGAATAATCCAAGATGCTCAGCTGAGCAGT
TAGGCCCTTCCCTTGGGGGTGAGTCCCCAACCCTGTCATATCTTCAACAC
CCTGTGGTTTCACTGGGCCACATATCACAGCAGTGATAAGAATGCAGTAA
TAACAGGAATGGCTAGCACTTTAGGAGTGCTTCCTGTAGGGTGCAACACA
ATGTTGCAATGATGACGTTTCATCCTGGAGACCCTGGGAAGTAACTGCTC
CTAGATAACAGGCGAGGAAACAGAGGTCAGCCTGCCGGGGCCCCTAACAG
ATGGCTGGGAGGGCGCCAGGGCTTGTCGTCCAGAAACCTCACGGGCCCAT
CACTCTTCCCCCAGGTGTTTGTGCTCTGCCAAGGCCTCCTGCAGCTCTGC
CAACTCCTGTACAGCGCCTACTTCAAGAGCAGCCTCACCACCATTGAGAA
GCGCTTTGGGCTCTCCAGTTCTTCATCGGGTCTCATTTCCAGCTTGAATG
AGGTGAGCAGGAGGTTCTGGTCCATCAGACTCCACTTGTGCACCACGCTG
ACCACAAGATCCAGGAGAAGCAGAGGAACAGGGGGCTGAGATGTGCACAT
AGGTGCAATGCCTCTTTCTGCCGGGTAACAGCAAACGAAACAGCAAAGAT
CCAGTGGGCACAGTGAGAGCAGCTAGATTTCCTGAGGCCTGACTTGTGCC
AGGTACTGAGCTGAAAGCCTCCCATGCCTGACCTGGGGGCCTCTTCATGG
CAGCCTTGCCAGCAGGTACTATGTTCATGCCCACTTCACAGATAAGGATG
CTGAGGCTCAGGAGTACAAGTGGCTTGCTCCAGGTATGCAGGGCTGTCCG
CCTTCAAATTCTGTGCTTCTAATTTAGTTCTGTGCAATCTGCCAGCCTGT
GGGGCCCCTGCCCCATACATAGTCTAGAGAGGATAAGCCCCAGGCATGCA
GGTGGGTCAGCAGCCCTGGGCCCAGGCATCTCCTGTCTTATGAGGGCCAC
AAGAGCCAGAGCTCTTAATGGCCTTGACTCCCTGAAGAGATCAGAGCTCT
ATGGCCTCCGAGCATTTCCTGCCATCCCTCAGTGACTGCATATCAAAGTT
AGAGGAAGACAAGATGGGCCGAGAAGCCAGAGGAGGTGGGAGGTCAGAGG
GCAGGGCCTGCCAGGAGCCACTGGAAGCTGGGGAGAGAGAGTGAGCGGAG
GCAGCAGAACCCCTTCATCCTGTCTCCATAGTCTGGAGACAGGGCTCCTG
GGCTGGCTTCCATCTTCTCCCCAGGGCCACTCTTCCTGGATGGAGGCAGG
CAGTGGTCCCTTCCCCAGAGCCGGGTTGCTCCTCTGGAGCATCATGAGGT
CTTCCCTTGACTTAGTGAAATTCAAGTCAGTGCAGTCACTTAGAGCCCGG
GCCCCTGTCCTGTGGCAGCTTCTGGCCAAAACAGAACTGTAAAAGAAGGC
AGGTATCTCCTCTCCCCAGCCTTCTCCTGCTGGAATAAGGCTTTCCCTTT
CACCCCTCCCCCTTCCTTGATGGTCCACATAGCAGTGACCCCAGGACCAC
TTTCCTGTGACAGTCCCTTCTACGGTCCTAGGAGGGAGGCACTCTTCACA
GTCGTGTTTCTCAGACAAGGAACCCGGCATCAGAAAGCCTGAGTACTCCA
TCCAGGTCAGCCCCCTGTCATGTGCATGGTGCAGCCAGGGTCTGAGCCCA
GGGCTGTCTGAGGGCTGCATGCCGAGGCCTCCTCTGTGGATTCCTAACTG
TCTTCTCTACCGTTCCTCCAGTCTCTTGCTCACAAGCACTGCTCCAGCCA
GTCTCAACCTCCACACACATCGCTGGAATCTTCATTGGTGCCCCTCACTT
CATGGGCACCCCTCCATGGGCTCTTTCGCCACATATATGACATGCATGCA
CATATACACGCACAGTCCTGCAGCCTGTCTAAGTCATCCCTGCTGGCCTG
AGACCTCCTCCTCTGAGCTTCCAGGCCTATGTGTGTGCCTTCCCTGCTTT
CTGTGCCAGGTGTGTGTCTGTGTACTGGTCTGTGTGTGTGCCTGTGTCTC
TGTCTGTCTGCTTGTATTGCTATCCTGAGCTTCCTGGTGATGCCTGGTGA
CTGGGTCTGCCTGACTGTGTGGTTGCCTCCTTTTCCAGGACAGCACCCTG
GTGTCCCTGCCCTGCATTCACACAGAATGACCCTCATCGATGTTGACCTC
TGACTTTGGTAATTCCCCTGGCACATGTTGTCCTCACTCATGATGGATAT
CAGGGAAAACAAATCTCCGACATTTGCGTCACCACGGTCTTGTTTCATGA
CCCTGGCAGGCATCCCTAATTACCCACCCCATCCCTTTCCTGCTGAGTTC
ATGCACAGCCTCAGAATCCTTCTCAACACAATATTCTAGGCCTTGCTACT
AATCAAAGTCAGAGCTGGCATGTGAGGTGAATCTTCTCCTTCACCTCAGC
CCAGGTGGTAGCCAAATCTGTGATCTCCCATGGTTAAATTTTCATTCATG
TTGACCCTATGCTTGGAACAAAAATGTGGGCCCGGAAACAACTGGATTCC
TCAATGGGCTGGGAGTAGCTAGCTAGGACTTGTAGCAGGATCTGCTTAAG
ACAGGAGGTTGATTAGGCAATGAAAGAGCTTAATATTTTCCCAGGGATGG
AGTTCAATCAGAAATAAAAATAATTCACCCATTAAAATCTTTCCCAGTCT
CAGATGAACTTTTCAAGAATCTGAATTCAGCTCATGCCTGTGGGCATGAT
TTGAAGCAAATATCATCAGATAGTTCACTCCTCTGACCTTATAACCCTCT
GTGTGTGTGTATCTGTGTCTGTGTATGTCTGAGTATTTGTGTGCAACTCT
GTATGTGTCTGTCTGTGTACAGTATATGCCTGAAGGCTAGGGGGGATGTG
AGAAGAGAGCCCGGCCTGGTGCAAGGGGAAGGTGAGGCTGCCCGGGTGGT
GGAGTGTGCGGTGAACCTCCAGGAAGCATGAACCTTCAGTCTGCAGCTTC
CAGTCTCAGAGAGCCTTCTGGGGGCCCAGTAGGTGAGGTGAAGCCTTTGA
CAGGCTGGGCGTAAAAGGCCAGCACCAGCAGGAGGAACTTAGTTAAGAAG
AGAAGACATGGAGCAACTTGGCTTGAAGCAGCAGCTTCTCTTGTCCACTA
CGAAAGGCTAGGGCTGAGCCCTAGAGGCTGCCTGCCCTTCACGCCACCCA
CCAGTTGTCCCTGCGGCCCTTTGGTTTGAGATGTTGAAGGGCAGGGGCAT
GTCATGATGGGCATTGTAACAGCTTTCAAAGAGTCTCACAGCAAGTCCAT
GGTAGTGTTTTAGGGGCTGCTCCAGGGCTGAGGAGACTGAGGTCTTGAGG
CTCTCTTCTCAGCCGGAACAGCTGCATTTGGGGGGTTGTTTTATATTAGG
GAAAAACAGTTTTTTCAAAGGGACTCTGTGGCTGAAGAAAAGTTTGAGAA
CAAGGTCTTAAGCCAACATTAGCTGTAACACCTGTGGAGGTTCAGCTTCC
CACAGAGAAACAAGTGCAGGCCCCTGGAGAGTGCAGAAGAGCTTCTCCTC
TGTCCCTCCTCCCTGAGATCCCCTTCGGGGCCCTCCACTGACCCTCACCC
AGGCCTTCTAAAGTCAGCCACAGTCAGCACAGCTGATATGATCTGACATT
TACAATCATTTTATGAAAAGCAATACGGAAACAAGAAGGAAATAAACGAA
ATCTGGGAAGCATAAGAATAAAGCTCTCTTTCCAGGTGTGGGTTTCAGGA
TGTCCTAGATCCACCTTTGGCAGGAGCTGCCTCTCACACACCTTCACATC
CCCTTTCTCTGTAGATCATAAACCATCTCACTCTTGGCCATGCTGGAGGG
GTCTCCTGGGCAACTATGTCCTCCTCTTGAAAAAGTCTACCATTTACTCA
TTCATTCATTTGTTCAACAATATTTAACGAGCACCAAGTATTAGGCACTG
TTCTAGGTGCTGGGGAAACAGGAATGAACAGACAAAAATCTCTGCTCATA
TATCATTCTAAGGATAAATATAGTATTTATGGAAATAAAACATACTGTAG
ATCAGGTTATGATGAGTGCAATGAAGAGAGAGAGTCATGGATGGGAAAAG
GTATACGGGGCCTCAGTGTTGGGGATGTAGGTGTATTTTAAATGACGAGG
TCAGAGAAAGACCTTCTTGAGATGATTCAATCTGAGCAGAGGCCTGAATC
TGGTTAAGAATGGAGTCCTGAGGACGTAAAGTCAGAAGGAGCAGCAAGAA
CAAAAGGGGCTGGGGGAATGCCTTTGTTCTTTGGTGTGGTCAAAGAACAG
CTGGGGGCAGTATGGCTGGATGGAGGGAGGGAGAGGAGCAGAAGAGTGCA
GACCTGAGAGGTGACCAGGGGGCAGGCTGTGTAGGACTCGGGAGGCTCTG
GGACAGCCTTTGCCTTTTTCCACTGGAGGGTTTGAGCTGAGGGAACATGG
TCTGATAGTTCAGCAGGACCCTTCTGGCTGCTGGGTGGAGAATGAACAGT
AGCAGGACATGGGTGAAGGCAGGGTCCCGTTAGGAGGCTACCACAGTAAT
TCAGACAGGATTGGAGGTTGAGGAATCACTGGCTTCCAGGTGGCTATGGA
AGGTCCTGCCAAGGGGATTTGCTGATCGATTAGATATGAGTGTGAGAGAA
GTCAAGATGATGTTCGAATGTTTTGTGTGAGCCACTGGGAGGATGGAGGT
GCCATTTACTGAGACAAGGAGGACCAGGACAGGGGACTGGGGTTTTGGAG
GAAGGAAGAAGAGTGAGAGTTTAGATGAGCTGAGACTGTGGGACTTAGAA
GCACACGCAGAGCATAAATAGCCATGGAACTGGACACACTTAGGGGCAGA
GCTCTGGGCCCAATGTTGAGAGTCCAAGGAGATAAAGAGGAATAAGCCAA
GAAGACCGAGAAGGAGCAGACAGGGGAAGGGAAGCAGGAGAGGATGGTGT
CATGGGAATTCTGAAAGAAGTGTTTCAGGAAAAAAGAAATGGTCAACTGA
GTCAAATGGAACTGAGCATTGACCAGTACATTTGCCAATGAGGAGGCCAC
TGGTGACCTTAGTGACAGCCATGTTCTGAGGCTCTAGCCTCTGTTTCCTG
ACATAGGAGTACCCGTTTCTCCACCCAGGAGGAAAGTGGGGTGTAACAAG
GCAGGGCTATGGCTGCAGATTTACAGAGCTCCTCCAGTCCAGGTGAACTG
TGCTCTGAGGAGAGGACCATCTTTGGTGGAGACGTGGTCAGAAGGCCTGG
CAGCATCCCCTTACCATTGAAGACCTTACTCCCCATGCTGGAGCCTTCAG
TTCCCTTCCCTGGTTCTGGGTGGGGCTCAGAGAGCACATCTTCCAGGGAG
CACCAGGAGCATGGAGTTACCCATGTTTCCAGCTGGAAGGGTTTTAGGGG
TGCTCTAGTTCTTATCCTTCATTTCACAGAATTGAAAACAGACCAAGGCA
GGGGAAAGGGCAGGATAGTAGCAGAGCTGCTTGGGCAGAAACCCTCTTCT
CCAAGAGGTGGCAGACAACTCTAGTTAAGCCTTGGTGTTTTAGAGTCAGG
CAAGACTGGTTCAAGCCATGCCCTAACCTCATAGTGATGGGTATGTTACT
TAATATTCTGAACCCCAGTTTTGTCAGATAAAATGAGAATTGTTTCATGA
TTAAATGAGATAAGCATGTTGCTTGCTTGTTATTCTGGCCACCAAGCCAA
GGTCCTTGGGTCCTTTAATTTGTACCTAATTAAACTTGGGATTTTGCCTA
AACTTTAGGTACAAACTCTCAAGCTTAATTAGGGAATGTGTTTTATTCCT
AGGAGCCAGCCAGAATTGGAGGTTTCTTTGGCTTTTTCCCAAAAAGGTTT
GGTTCTAAGATGGTTAAAGTTTAGGTTTTGAGTGTTTCATAATCTCCAGA
TACTTCCTTTGGTCTGGTCTAGGAGATGGAGACCCAGAAGGGTTTCTGAT
AACAGAGGGAAAACCAAAATTGCTTATGCTTGCCCCTTGTGAAGCTCTCT
GGGAAAAGCTGACACTGTAACTCTGTCTGGGCCCCAGCTGACACTGGGCA
GCCCCCTCCCTTTCCAGTTTTCCTGGATGAAGTGTGATCGGGTCTTCTCT
GTGGGTGGGTGAGGACAGGGCCTCTTGTCTCACTCAGCCCTTCTGCTCTC
TTCTCAGATCAGCAATGCCATCCTCATCATCTTTGTCAGCTACTTTGGCA
GCCGGGTGCACCGTCCACGTCTGATTGGCATCGGAGGTCTCTTCCTGGCT
GCAGGTGCCTTCATCCTCACCCTCCCACACTTCCTCTCCGAGCCCTACCA
GTACACCTTGGCCAGCACTGGTGAGTGGCTTCCAACCCCTGCCTCAAGGG
GTAAGGAATGGGTGAACATCTCACAAAGTTAGTGGGGATACTCCTAGGGC
AGTGGATACCACTCCAGGAAGAGGGCATATGAAGCTCAGACTATACTGAA
GTATGTAGAGCTATGGAGCAACTGAACTCTGGTTTGTTCAGGAAAGTGTG
CAGTGCATGGCTCACCTCCTGCTGTGATTCCCTGTGCTTCATGCCAGGAA
TGACCCAAGCTCCACCTCCCTGGGGACACTGTGAGCAGTTTCTGCAATGG
GGCTTGGCAGGAGGAGGATCAAAGCATTAAATCATGATGAAGTCTGTGGA
TTTTGTCCATGTTTAATCATAGCACTTCGTGACATTTGTACATTCTTCTG
TCTTTAGGTGACAGTGCACTTGCACGATCTCATTTGATCATGTTTACTAT
GTTGAGTTTGCCGATCCATGAACATAGGATGCTCACTATTACCCTTATAT
TTAACATTGATGTATGACTTAACAACCACTGAAGTTCATTAATGTCTCTA
TTCTCCAAGAGAGCAAAATAAGGATCATAGATGCTTTTTTGCCCACCATC
CTCTCTCATCTTCCACTTATGGTTGACCCATATTTTTGTTCTACCTCTCT
TTAACCTCCCCACAGTGGTCATTATTATAAATTGTTACTATACTGTTATT
TATAAATAAGTGAGTGGTTATTTATGTACTCACAAGTTTACTGGTTTCTT
TGTTCACCATTGATTGCTTCTTGCATTTCACCCATGCTGTTGATTAATTT
TTTTTTCCTGAAGGATATCCTTTAGTGTTACTTTCAGTAGGCCTATGAGT
AAGAAAATCTCTAAGTCTTTGTCCGAAAATGCCTCTATTTTACCCTTGCT
CTTGAATTATAGTTTAGCTACAAACAGGATTTTAAATCAATAGTTATTTC
TTCTCAACACTTTGAAGGTACCATGCCTCTGTCTTCTGGTATTGGGACTC
TTCCATTAGTCTAATTGTCATACCTTCTTAGGTATCCTGTTTTTTCTCTA
GTTCTATTTAGTATTTGTTCTTTTCTTTTCTTTGTTGTTCTGTATGTCTA
CCATAAGGTATCTGAGTATGGCTTAATTTTCACTTATCTGTTAGAAAGTT
CGATGTGCTATTTCTTTTGAAAATTAGAGTTTTTCTTTAATTTTGAAAAA
TTTTCTGCCAAAAACATTCCTGATATTGCACATCCCTGTTTTCTCCATCC
TTTTTTTCTGGAACTCCTTTTACATATGGCTTCACATTTTCATTTCTGGC
CTCCTTGTCTCTTAACCTCTCTTTCATAGTTTTCAGTGCTTTATGCTGCA
TCCTGGGTAATTTCCTTAAAGTATGACTTCCTGTTCACTTAGTCTCTCTC
CAGCTTTGTTAATTGGTGTCAAACCCATTTTTTACATTATGAATTTGAAT
GGCACTACATATTTTGCTAATTCCTGAAGTTCTTATAATAGTTGTTCAAA
TCTCTCTGCTCTGTGACACTATTTTTTGGTGTTTCCTTATGATTTCTTTT
GTGTTATTGATGTCTTCAGCTATTTTAAATATATTTATATTATACTCTTT
TTCAGATCGTTCTAGTAGCTCAAGTTTTAAGCATGTTAATCCTCCCATTG
TTTTATGTCTGCTTGCTCTTGTTCATGGTGGATTACCTCCTTGAGTTTGA
CCATGAGCCATTTGCAGTGGGCTTTTTTGCCCTGTGGAAATTGTATGTGG
CCTGGGTAAGGAAATAGCCCTACAGAGTGGTTTGGCATTTACATTTACTC
AGCACCCAGGACCGTCATTGCTTTGGAACTAATTTTATGTTAATGTGTTG
ACTTAGGATGAATTTGCACTTGTTTTAAGGACTGTGGAAAGTGGCTGTGG
TTTTGTGATAGACATTTGTGAGAGGCCTAGTTCATAGATGCTCACCCTTA
GTCCACTGTATTGGAGTTGAGGTTAAATGAGCCCCCAGGGAGGTGCTGAG
GGAATAATCGATTTTGGTTTGCTTAGAAACCAGGCTATTTTTTCAATCAG
ATATGTAGACCAGGTTTGAGAGATGAACCACTTAGAATAATAGAGGGCAG
CCCTTAAGATTGTCAGGCCAGTGATGGAGAGAACGAAGTGCCTGGAACAG
GAAAAGAATAGGGCAAGAATGGGGGTGTCTAATGATGAAATATCTGCTGG
GTTCTGGTCATGGAACTCTTTCCCCCACCTTTTTATAAATCTCCAGTGAA
CTGGTCATTTCTCATAAATGTATGGACTCTGTGGGGCTGAATCCTGCTTC
AGGTGGGCAGAGGATGAGGAAAGATCCGAGCCCCAGGCTCCTGCCATACA
CTTTCTCCCCCTTGGTCTAATGAGAGCTTGAGGCCAGCTCTGAAGCGAGT
CAGGAGTTGCTGGCCATCTTCTCAGAGAGCAGTGTGGCCAGCCTGGCTGC
ACTGTCTTCTTTCCAGAGCCACAATCCCTGCCCTTTCTCGTCACGTGAAG
ACTGGGATAATGAAATCAGCAAGGGAGCCTGGGGCACTGTGGCCAGGCCG
AGGGGAGGGCCTCCTGCCAGGCGCCACACATCGTGGCCTGCTCAGCCTGG
CAAGTCAGCTCCCATGGCCTGGGCCCACATGCCAGTGGCATTTGCCTCAC
ACTGAGCAGCCACAGGAGACTGGTTAAGACAACTTCACTCTGTTCCTTGT
TAGGAATAATCAGTCAGTTTGGGTTCAGCTAACCATGACCCTCTCAGCTC
AGAAGGAAACACTGTGCTGTTCCAGCCTTGACCTCAGTGCTCTAAGACAA
AATCTTCTGATAGAGGAAAGAGGGGCTGTGAAAAGACCACAGCTTGCCTC
TCAAGGCTGCAGGGGCGCACCATAGGCCAGAAGAGTGGGCTGTTTTGTAG
TTCTTGTTCATTGGTGCAAATTCTATATGCAACAATAGGGCACAGCAGTC
TCCCTCAGTCTGGCTGGGATAGAAGCAGATGCTGGAAGTGCTCAGAGCTG
AGCTACCTTCGGGCATCTTCCTGGTGCCAGGGGGTGGGATGAGAAGGACT
TTGGCCCAGGAACCTCATCCATGCGATCTAGCTGGGGCTGCACACGGTGC
CAGTGCACCACGTGAGTGTGCTATGCTAATATGTGCAAATAAAAATATTT
ATAAAGTCTTACTTCATGGGAATAGCTTAAAATGTGACTCCTACTTCAAA
TGTATTCTTATGGAAATTTTAGTTTTGAATCATGCAAGGTCGTCAGGAAC
AAATGCATTTCATTAAATGTACCACTCTGAGTGGGGGTGTGAACCTGAAT
GGTCTACTGGAACCAGGCAGTCCTCAGTCTGAATCTGAGCTATGGGACTC
CCAAGTGGGCTCGAGCCACCTCCTCCCTGCTCTGAGTTTCAGTTCATCCC
TGAGTGTCTATTTTATAGCCCTCGTAGGGCTGCAGGGGCTGAGAAGGTGC
CTGTTCAGAATTTAGCCTGGTGGCTGTCACATGCAGTAATCACTAAGTGG
TGGCTTTTGTCCCCACGGTGGTAGGGACAATACATGATGTTGTGTAAACT
AGTAGACTTTCTGGATCCCAAAGGAGAAAGCACCTTACTTCCTGTGAAGA
ATGTGACAGCTTATGGGCTGGATGGCCTGGGGACATCTGAAGGCAGCTTC
TTTCTGGTCTACATCATAGGTCCTCTGCTCTGTTCTGGAGCAGGGCGCCT
CCCTTGGCATGGTCCCTGCTGCATCCTCACATGTGAGTCCTTAGGATTTC
TGTTGGTGGAAGCTTAGGTTGCTGTTGTGCTCTCTTCCCAGATTCTTGTG
TTGGTGTGGCCTGGTATGATGGGTAGACATGCACCCCACCCCACTCCATA
TGGACTTTAACATTTGAATCCTAGCTCAGTCACTTACCTGCCAAGTCTTC
TTAGGCAAGCTACCTCACCTTTCTGAACTTTAGTTTCCTTACCTATAAAA
TGGGAGCAAGAATACCTTGACTTTCAGTGTGTTCTGAGAGTAAAAGTAAA
ATGAAATAAGCCAGGTATGCTGTAATCCCATCACTTCTGGAGGCTGAGGC
AGGAAGATTGCTTGAGGCCAAGAGTTCGAGACCAGACTGGGCAACACAGT
GAGACCCCATCTCTACAAAAAAAAAAAAGTTAGCCGGGCATTATGGCACA
TGCTTGTAGTCCCAGCTACTTGGGAGGCTGGGGTGGGAGGATCACTTGAG
CCCAGAAGTTTGAGGCTTTAATGAGTTATGATCATGCTACTCCACTCCAG
CCTGGGCAGCAATCAAGGCTCTGTCACCAGGCTGGAGTGAAGTGGTGCGA
TCTCGGCTCACTGCAACCTCTGCCTCCCAGGCTCAAGCAATTCCCCTGCC
TCATCCTCCCGAGTAGCTGGGACTACAGGTGCCCACCACCAGGCCCAGAT
GATTTTTTGTATTTTAGTAGCTACGGGGTTTCACCATGTTGGCCAGGTTG
ATCTCGAACTCCTGACCTTGTGATCCACCCACCTCGGCCTCCCAAAGTGC
TGGGATTATAGGCATGAGCCACTGCGCCTGACCTGCTCAGCATATTTCTA
TTTGCCTGCCCTTGTTGTGGTATTTGAAATCTGCTACTCAGACCTATTAC
TTGTGTTGATTACTGATTCCTTCCTCTTGGAGTAAAGCTGCTCCAGAAAC
GAACATGGGAAAGGAAGAGTAGCAATGTGTTTTAGGAAAAAAAAGTTAAA
GAAGGGCTAAACATTTTGGGAATCACAGAGACCTTTAACAACTAATTGCT
AATTTTTAAAAGGCCATGAAGACATTTATGGATATTTAGATTTTCTAAAT
GTCAGTGGGGTCCGGAAATGTACCCTGGCGCCTTTAGAGAAAAAACGAGC
TATCCAGCCAAAGCCTCTAAGACCACGTCTGGAAGGCGCGGAGAGGTTTT
CCTCTATGGGAAACTGGCCAACGTGAGACCAGTCCTCCCAGGAGATAAGA
GAAGGGGCTTCGGAAACCAGTGTCTGGTAGGACTAGGTGTAACCTGTGAA
TGATCCTTCAGCCAAGTCCAGAGGGAAGTCTGCCCACATGCAGACAATGG
ATGCCGCAGGCAGGAAGCTTCCTGGAGGTTAAGGAGGGGCAGGGGAGCAC
TGTATTTTTGGAGCAGACAAGAAAAAGAAAGGGAAGAGTCACTTTCCAGT
CCTTTATTCATGAAGTTGCCATCCCCAACCTGGGCTTGCATATCCCAAGG
GTGTCCCTGGGAAAGAAGGCGGTGGGAAGGCACAGGGCCGGGGAGCCTCG
GGATCCTGAGTGAACTGGTGAGGACAGGGAGCTCAGCCTTTCCACAGTGA
ACACCCACCTATGGTGTGAGATGGGTGTGCTAGGAAGCCCTGGAGGAATG
AGAGGGGCACCCGTGGTTTGGGCAGCCTGAGTTTGTTTGTGGGTAATTGT
GTATAGGTGTGTATGTGAGGGAGGCGTGTCTATGAGTAGGAAGCTAAGTA
GGTGAGGAGGTGTGTGGGTGCGGAGGGGAGGTAGGTGTATGTGGAGATGT
GGGGGGCATGTGGGGATGGGAAGGACTTGGGTAGGAGGCAGAGGAGAGTA
AAGTATGTGGATGTGTGAGTGGAGTTGTGGGGTGAGGGTTGGGGTGTATG
TGAAGGGGAAGGTGTGAGGGAAGAGGTGGTGTGGGGGAAATGTGTGGAGG
TGGGGTGGGGTGCCTGTGATGGGGAAGTGTGTGTGCATGTGTTGGGGGGG
TGTGGAGGTGAGGAGGGGTCCATGTCGGGGGAAGTGTGTTCACATGTGTG
GGGGGTATGGAGGTGGGGTGGGGTACATGTCGGGGGAAGTGTGTGGGGGT
GTGGAGGTGGGGTGGGGTGCCTGTGAGGGGGAAGCGTGTGTTCATGTGTG
GGGGGTATGGAGGTGGGGTGGGGTGCCTGTGAAGGGAAGCATGTGGGGTG
GTAGGTGATTGGAGGGTGGGGATGGGCAGATGTGCAAGGAAGGAGGAGAA
AGGGTGTATGTATTGGTGGAGGGCGGGGTGGAGCATCCTAGTGGGAGTTG
CACAACCTGGCCAGTGGATCTATTTAACTCAAAAAGAAAGAGAAAATAAA
CAACAGAGAAAATCACAAAACACATGGGTGTTATTAATTTGACTGAGAGA
AATAGAGCCCTGAACGGATGAGCAGAACAAAACTTAATGAGATTTGATGT
GGAACATTTTTTAAATGCGTGGTGTAGGAAAGAATAGAAATAAACATGGA
GAAATGATAGAAAACACTAGACTGCGGTAGAGAAGGCCCGGGAAGGAGCG
AAGCTGGAAACTGGTGGGAGAGCTCTGCGCTCCATGGTGATGGTGTGACA
GCCTCTCTGAGGGCCTGAGCCTGAGCCGTGACATAAAAGTAGCTGACTCT
CTGTCAGGCTTTTCTATCATCTGTTTTTGTGGGCAAGGCACAACTCCCCT
TGGCTTACCTGGAAACTGGACCCCTTGTTCTGCGTCTCTAGCACAGGCCT
GGTGGAGACCTAAGTGTGGACCCTCAGGCATGCCTGATCATTTCCCTTGC
TCAGGAATGAAGCTGAGTGACAGGTCTGCTTGGGGGGAGGGTCCAGGAGG
CAAGCTGCAGCCTGCTCCGCTGAGAGTCGCTTCTCCAGGAAGGCTGGCAG
ACTGTGTCTTGGGCCCACCTGTAGCAGGAGGATGGCCTCCTTGGGAGTTA
GTATCCAGGGTAAAAAGGAAAGCGGGCTTCTGTTCCAGTGGCTTTGGGCA
TCCTGGGAACAGTGTTGAGAATGAGGGAAAGCCCCACAGAAAAAGGGAAA
TGTGGTTCAAAGAAACCATTGATCCTCCCTGCAAGATGCCTTGAGGTCCC
CAAGCATCCCCAAGGGAGACAGGGAGGAACTTTCTGCTCTTGCTGCCACA
CTTAGTAGAGCTGTAGAGCTGGGACACTGACTGTGTGCACAGGCATCTCC
TGTGGCCCTGAAGCCTTGAAATGGGAACCACTGGGCCCAACTGGGCAGCG
TGCTACCCACCAGGAGGTTGGAAACAACAAGGAGAGTTTCTTTCTTTCAG
GAACAAAAGGACTTTTCAGGATTGAGAGCTTGGACTGGACACAGTCAGAG
AGGATGTGAGCCTCAGAAATCTAAAAGAGGGAAACAGCAAAGAGAAGTTA
GCGTGACAGAGGACTGACCCACAAACCAGGACCTAAGGGGGCTGGCTGGT
GCTCTGGAATGTTCTGGAGAGCTCCTTCCTGGCCTCTTCTTGGCCTGGGG
CTGCAGTGCGGAGAGGTGTGAGCAGGGGACTCCATACTCCCCTCTCATCG
TCTTGTGCTGGTGACACCTGCTCTTGTCTGTCTCATACATTGTGCTTCTC
CAGAAGCATTGGTTGCAGAAGATTTGGTGGCTGATGTGTTTGAACACCAT
GGGACTAGACCAGGGGTCAACAGACATTTTCTGTAAAGGGCCAGGTAGTA
AATATGTCAGACTTGTATACCATAAGGTCTGTGCTGAAATTATTCAACTT
TGCTGTAGTGGCACAAAAGTTCCCCTTCCCAATAGATAAGCAAGTGGGCG
TGGCTGTGTCCCCATAAAACTTATTGATAGATACAGGCTGGGAACTGAAC
GCAGCCCACGGGTTGTGGTTTGCCAATCTCTGGACCAGACAGTTCTCTCT
GCCAGGCCCACTACTGCCCTCAGGCATCTGGAGCTGAAGCTCAAGAATGG
GGACTGTAAGGCTGCCTGAATGTGGACAGTCATGGTTTGTGGGAGCAGGC
CCCTCCCTCTCTCCCATGTCACTCGTTTTCCTAGATTTCCCTGAATTCTC
TCTTCCTATACTCCACTCCATCAGTCCCAGGTCAGTCTTCTGGAACCAAG
CAGAACCAGCCCATCAGTCCACAGTGGTGGAAACAGCCATCACATAGCCC
AGAGCCTTTGCTTCCTGAGCTGCTGGTCTTTACACAAAATAAGGCTTCCA
GCCCTTTGGGGGCCCAGTGCGGTGCTGTCAGGGTGGTCTCCCTGAGAAGA
AGACAGAGGGAGGCTCACATCTGAATCTGAGGGTCCTCCTCACAGGCTGG
AATTGTGAACTTGCGGTCAAAACACCAGAGACTTGTCCCCATACATTGTG
CCAAGCTGACTAGCTGAATTCCTTGAAGCTACGGTCAAGCTTCATATTTT
CTCCCTGTAGGAATCATTCTCCTTGGTTGGGGTCCCTCCTTATAGCCGTG
GGTCTAATTTTGAGTTATCAGCCGTTGGACTGTCCAATCCTCCCAGCTTC
GTGTCATCCATGTCATTCACACATTTGTAAGCAGTTTCCTTCTTTCTCCA
AAGTCCGTGACAAGCGTTGAGTGGCATGCGCTCAGAGCAGCTTCTTTCTG
GGTGACGCTGGCCCACGGGCACCCGAGCCGGGCATGCTTTGAGCCAGTTT
TCCCTGAGGACTCCCCAGCCATGTGTCCTCATCTTGTCTAGCAGGATGTC
ATGACAGGCTTTGTCAAAAGGCTTCTTCCAATCAAGTAACACCATGTTAT
GGCTTCCTGCTGGGTCAGGAGCCCCATTAACAAAGGAAATGAGGTTGGTT
TGGCAAGACTGGCTTTTGATGAGCTCATGTTGGCTCCGCTAACGGTTTTT
TTTTCCCCCTTTAAAGAGCTTCTCATCAGCTTACTGAATAAATCTGTGAA
CAATAAATTCTATCTCACACCCATCAGGGTGGGGGAGTACTAATTAAGCT
CACATTTCTTTGCATGTTAAAATTAAATGGCTCTGAGCTGCATCCTGCTC
CCTCAGGTATGTCTTCCTTCCCTTCATCCACTAAAGAACCAGCTCTGATG
ATGAGCCGAGCCAGGTGCTGGTGCCAGACCATGTGCTGGGCCAGGGTGTG
GGGGTGTGGGCAGCGGAAGAGAAAGCCCCTGGCCAGCAATATAGCACCCT
GCCTTGTTGGGCATGCACAGTGAGCCTACCGTCTTGTGCCCAGCTCATTC
ATAGCACAGCCTCCTCAATACAAGTAGGCATTTTCCCTAGGGCCCCCTCT
AGTGCCAAGGTGGCAGTATGGGAAGGGACATCCAGCCTCAGCCTCAGAAC
TAGTCTTGGAAACCCAGTCTTGAGAGAGAAGCAGAGCAGTTCCTCCTTCC
ACTGCCCTGAGCTTCTTTTCCTCCACAGATGTGAACTTCTCCAAGCACAC
TCACTTGTCCTGTAACTGCTTTATTATAATCCATCCATTGCTGGTGACCA
GCATTGATCTTACTGGTATCTAGTTTCTGGACCCAGTTTCCAAGTTCTTA
AAGGCCTGGGCAACATTGCCTGTCTCCAAGCTTCTCCCATTTCCCTTATC
TATTTGAGTTTCTCACAGTTACTCATCTCTTTGGATGAAAATCGATCTCA
TCGTATAATCACTATGTCCCAGGTTATAATATACTCATTATTTTTAATTC
TCATAAGAATATTGTAAGACAGGTATTATATCTGTTTTGAAGACGAGGAC
ACTGAGGTTCATTCCGAGAGGCCTCTGACTATCAAGCAGTAGAGCCAAGA
TTGGAACCTCATCTGTCTGGCTTCAAGGTGTGAATTTCTTCCATTGCCCC
ACAATGTGCCCCAGACTGTCAACGTCAACTCATTTTTTCCTCTGGGGCAG
AAGACTATGTTCTCCTAGTTTTCCAAGATGAAACTGAGTGCATTTGCCCA
TAGGAATCATTTTATAAATGGAGGCTTAGTTTAGCTGGAATAGAACACAT
GTTATTACTGGGGCATTATGGATTAACTAGGCTTTGTGGACTACACTGGA
AACCCAATCATTTCTGATTTGCAAACAACCCAATTTACAATCGACTTTGG
GATCTCAGATCATGAGTGAGTTTGGGACAGTCTGTTCCAAGCCTGCCAGT
GGGTGTTGCCAGAGTTTCAGGAGGAACTGTAGAGTAGCTGAATTTAGGAG
ATGAGGAAATAAGTCCTTCAGAGTGGAACATTCTGAATGAGGTTCCAGGA
GTCGTGCTGATAGTTTAAGGGCTTGGCACCTTGGCCATGATTCCAGATAA
CTCTCTGGAAACACCCCTGCCTTTCTCTGTTGTTCTCTTATGGATTGCTT
CTGAAGTTTGGGGCTTGCTAACCTCTTCCCCATTTGACCCTACTCACCCA
CACACTCCCAGATGACAGCCATGTCTCCTTCTGAGCAGGGAGGGGATGGG
TGGCTCTTAAGAGCCATGACTTGAAGGAGCCCTCCTCTTTCCTGGCAGAA
GTCTCTCTCTTTGCTTTACTAGAATGATGAGAATGACTAGTCAAAGGAAA
GCCCCTCTTGCCATGGCCTTCTCTTGTTGCTTGGTCGTTCTAGGGTCCCC
TCCCTACCCCAGCTCTAGACCTTGGTGCAAAGCCCTCTTACTATTTATTC
CCTTGACAGCTGGAAGGCTGGCCTCAGCCTGGCCATCCCGAGGGGTGGGA
CGGAGCGCTGAAACATGTGGCTTTGAACACCGGCATGGCTAGACCTTGGC
CTGCCCCAGATCTACCCCATCTCTGGAGGTTCCTTTCTAGGGAAGTAGAC
CTTCCTGACAGAGACTCCTACATTTCTAAAGCTGCTGAGGGTCCAGAATT
TAGAAATGAAATTCTGCATATTTAAATGGTGGAGTGAGTCAGAGGGTTGA
CTCACATGCTGGTGGGGTGGGGCTGGGGGAGCTGGAGGAGGGAAGAGGTC
CGGCCTGTTTCTAGTTAACATTTCACAGCCCTGTGAAAACTAGACTGGCA
AAGCCAGCCCTCTGGTGTTCTATGGGAAAAAATGTGCTCTGATGAACTTG
AGCCTTCCAGAAGGTTGCCTGGGGGAAGATGAGAAACAGACTGATACCTG
GCTAAGAAATAGACGCCCCTCCCTCCTCCAGACCCCTCCAAGATAGGGGC
AGGTGGCTGTGTTTGGGCAGGCAGTCTCTGGAGGTTGTGGTAGCAGGAAA
AGGCAGGCATGTTCCTGACCTTACTACAGGATTTGACTTGGTGGAAAGGT
CAGAGTTAGCAACCTCTAACCTGCCATGCTTCTATAAGTTACGTGGAGTA
GCTGTGTTCCAGCCTTTACTAGAGTGCTTGCGAAGCAATGAGAGGTTGCT
GTTTCTTCCTAATGTGTGCCCGAGTTCCTGAAAAGGCTGGAGCTTTTGCT
CCAGGGAGGAGAGGTGTGGAGTGGTGCAAAGAGACTCAAGTCCCCCATTC
TGTCTGCCAGAGGCTGTCCATTATCAATGTCGCTAGATCAGAGGCTGCCC
ACGGGGTTGGTGCTCATACAAGGTCTTTTTCTTCCCCTTCTTCCTGCCCC
TTGAACTCCTATCCCTGTCCTCCTATTTTTTTTTCTTTTTTAAAATGTGT
GTCCTCTTATTTTTATAAGGGAAGAGCTTAATGTTCTAAATTCTAGAAGA
CTTGGCTGAGCGCAGTGGCTCACGCCTATAATCCCAGCACTTTGGGAGGC
CGAGGAGGGTGGATCGTCTGAGGTCAGGAGTTCGAGACCAGCCTGGCCAA
CATGGTGAAACACTGTCTGTACTAAAAATACAAAAATTTGCTGGGCGTGG
TAGCATGTGCCTGTAATCTCAGCTACTTGGGAGGCTGAGGCAGGAGAATC
GCTTAAACCCGGGAGGCAGATGTTTCAGTGAGCCAAGATCATGCTATTGC
ACTCCAGCCTGGGCGACAGAGCGAGACTCTGTCTCAAAAAATAAAATAAA
ATAAAATAAAAATAAATTCTAGAAGACTCTATTCTGACCAGAGTGAGCTA
GCCAGGGCCGCCCACTGCCAAGGGTGTCTCAGGCTTGATTGTCTGGGAAG
CCCACACTACATCTGTCTCTGACATAAACACCTGTCCCCGCATAAGGCCG
GTTCTGTTAGTGGGCCACAGCTTCTCTGATAGCACTGACCTCGGGGTTGC
ATTTAACTGCTGTTCCCTTTTTTATATGTGTCAGAACTTGATCTGGGATG
TGGGATGGGTTTGGGCCCTATTGAGTTGGCATAAGGAAGAAAGTTCTTTG
GAAGATCTTGAATCAATTTGGAATGTAGAATAGGCCAAGAGGAACTCAGT
AGACTTGGATGTAATTGGGGCCCATTGATTATTTTTAAATACATATTTTA
ATAAAGGCAGTTTCTGACATGTAATCCGACATTTTTAAAGTTAGTGGTAG
CAGTTAAACAATCCCCAGAAAGTTTCACCTCCTGAACACTGCATGAGATG
AACTTCATGAGGATTTTTGTGGCCGTGGTGCAGCAGGCAACATGCCGTAT
GAAGACTTAATAAGTCATTCAATGTTTTTACCATCTTTGATGACCATTAA
ATACCTTGCAAAAAATGATTTTGCTAGTCAAGTCAGCAACAATGTTCAAA
TAAATATGTATTAAATAAATTAATGAGAATCAGTAGAATCAAGAACTAGC
ATTCAAAAAACCCACACATTTTCTGGCCCTTTCTTTTCCCGGTGATGTTT
CTCATGTCATGCCCCTGCTATCCTACAGCAGCCTGGCCTTGTGTGTCTAC
GCACACACCCTCTGGGCTATGCATTCACCATCCTCGCAGCCACTAAGCCC
TGCCCCTCATACTATGCATTGACTGCTTCTTGATGGAATCAACTCCCCTC
CTTACCCGTCTCAGTTGACAGGGCCTTGCACATTACTCCTAAGTCCTCTA
GTGTGTTCATTGCACTGACCCCTCATGATTTTCACCCTTCTGGAGTTTTC
CTTAGGATACAATCCAGGCCCCTTCCCATCATGCACAAGTCCTCCAGGGG
CAGCCAGGCCTCACCCTGGAGGACTTGTGTTTGAGCGCCAGCCTCACTGA
CCTTTATTCATGTTGTGGAATGTACCACCCCCGCATCTGAGCCCTTCCAC
ACTCTCTTTCTCTCCTCCTCACCTCCCCACTTCCCCACCCTTATCTTCTA
TCTATGCCTGTCGATTCTTCTCAGTTCAGAGGCCACTTTCTCCAGAAACC
TTCTCCAGAATGTCCTCTCCTACAACACTGGGTCTGCTGCCTCTCTCATA
TGCTCCCTGGCACCCTGTCCCAGCTTCACATGTCCTGTGCAATATTTTTA
AATTGTGGCAAAATATATATAACATAAATTTTACTGTTTAACCACTTTTA
AGTGTATAGTTCAGTGGCATTAAGGTACGTTCACAATGCTGTGCAACCAT
CATCATCATCCACACCTGGAACTTTTTCATCTTCTCAAACTAAAATTCTG
TATCTGTTAAACACTAACTCTTCATTCCCATCTCCCCCAGTCCCTGGCAA
CCACCATTCTACTTTCTGTTTCTGTGAATTTGACTTCTTAGATCCCTCAT
CTCAGTGAAATCACACAGCATTTGTCTTTCCGTGTCTGGCATATTTCACT
TAGCATAATGTCCTCAAGGCTCATCTGTGCTGTAGCATGTGTCAGGATTC
TCTTCCTTTTTACAGCTGTATAATATTCCATTGCATATACCACATTTTGT
TTACCCATTCATCTGTTGATTAATATTTGGGCTGTTTCTAACTTTTGGCT
ATCAGGAAGATAGTGCTCTGAATATGGGGGTACAAATATCTCTTTAAGTC
CTTGCTTTCAATTTTTGGGGGTATATACTCAGAAGTGGAATTGCCAGATC
ATATGATGATTCTATCTTTATTAGTATTTATTTATTTATTTATTTATTTA
TTTATTTATTTTTGAGACGGAGTCTCGCTCTGTCACCCAGGCTGGAGTGC
AGTGGCGCCATCTTGGCTCACTGCAAGCTCCGCTTCCTGGGTTCACGCCA
TTCTCCTGCCTCAGCCTCCCGAGTAGCTGGGACTACAAGTGCGAGCCACC
ATGCCCACCTAATTTTTTGTATTTTTAGTGGAGACAGGGTTTCACCGTGT
TAGCCAGGATGGTCTTGATCTCCTGACCTTGTGATCTGCCCGCCTCAGCC
TCCCAAAGTGCTGGGATTACAGGTATGAGCCACCGCACCCGGCCCATTAG
TATTTATTTTTTTTTGAGACAAGATCTCACTCTGTCGCCCAGGCAGTGGC
AGGATCTTGGCTCACTGCAGTCTCTGCCTCCTGGGTTCAAGCCAACCTCT
CACCTCAGCCTCCCAAGTAGCTAGGACTACAGGCACGTACCACCATGCCC
AGCTAATTTTTTGTATTTTTAGTAGAGACAGGGTTTTGCCATGTTACCCA
GGCTGGTCTCGAACTCCTGGGCTTAAGCAATCTGCCCCTCTTGGCCTCCC
AAAATATTGGGATTACAGGTGCGCGCCGTCACGCCCAGTGTTGATGATTC
TATTTTTCAGTTTTTGAGGAACCACCCTTCTTCTTCCCACATTGGCTACA
CCACTTTACATTCTCACCAGCGATGCATAGGGTTCTAATTGCTCCACACC
CTTGCCAACACTCGTTTTCTGTTTTTTTTTTTTTTGGTAATAGCCATCCT
GATGGGTATGAAGTGATATCTCATTGTAGTTTTGATTTGATAAAGCAATA
TTACGGTTGCTTTTTTGATGCTCTGGGTACCACAGTGGACCACAGGGTTT
GTTAGGCACAGTACCTGTGCCTCTCAGTCATGCCCACATCTCCCACCCAA
GCAGAGTGCCCACCCACAGCCACTATCAACCAAATAACTGAGCGACTGAA
GAATGAATGTAACTGGGTCCAGGTCCTTTTCAGGACTCCGCGTTCTCCTT
TTCCCCTGTGACGCAGGTATCACTCATACTGCTGCTGAGGGGCAGCTGCT
CAGACCTAGGCATCTAGGCATGGTCCTTCAGTGGCTGAACTGGGAACAGA
GGCTGATTCTTTGCATCTCTTTCCAGGTATTGTGATTTGCTTTAAACCGT
GCTCCCACATAACTTAGACTTGGTCCTGCCACCCCACGGCCTATGAGACA
ACGTGCCACAGAGAGCCACTGTCACACCGAGGGGAATGGCTCAGCCAAGC
ACACTTGTGAATGACAACAACCTGAGGTTTTAGGGAACTCAGACTTTGTT
TATTTCTCCATACACAGTGTACCCAAGATTTGTCTGAATCTTCAGAAATA
AATACCCACTTGGAAATGTAGCAGCAGCCCTTTTATTTATGGATGCAAAG
TATATGTCCTTCAGAAATGCATCCGTTTGGTCAAAGTTGGCACAGAGAAG
TTAGCAGAGAAGTGTGCGTGTTGATTTCTTTGAGTGACTGCACAGCTGTG
TTGAGTCAGTTAGCCATGCCAGGTCAGGTGGGTTTGTATAAATGTTTGAA
CACTTCCCAAGCTGAGCAGCTGGCTTAGCTCTCTTTCCCCTACATGCCTA
TTTGGTCCTGAGAAGAGCATGGGGCCATGTTACCTGTCCCTCACTCCCTC
CAAAGACCAGCATAGCCTGTGCATATAAACTCTTACCCACCTGGGGCAAC
AGCCCCGGGTGTCCTGTCTGGCTGCTGCACTGGGGGAGTGACCAGTGAGC
CTAACACTCTGCCCACCAGATCCACCAGCAGCCTCTCACCCTCTGCTGTG
CTTGGTCCTTCCCTGGAGTCCTCTGAGAGCTTTCAAAGTCAAAATCACCA
ACATAGAGAGCCACGAATTCAGGGTCATAGGAATCTGGGTCCCAGGCTCC
CAGTCCAGGCCTTCCAACATCTGTTAGCCTTGCCCACTCAGAGCCACCCT
GACCTCACCCTGGGATCTGCAGGAGTCAGCCAGCGCCACACCCAGTTCTG
GGTGTTGGAAATGGTCCATTTGATAGATTCCTGAGTGGGAGGGTGGGCCA
GACAGCCCCTGTGGTCTTTCAGCAGGAGCATTTTACAATTCTGTGCTTCA
GGACCCTTGCAGCATCACTGAGCTGCACCAGCCTTCTTTCAAGATGGCAG
ACACAGCCTCTAGACCTGGTGTGGGACTGGCAGGGTCTGACTTCTCATGC
TTGGGCATCTTTTTGGAACCATAGTCTTCTACCTCTTTAATGACACAGAG
AATCTTTCTATCTAGCCACCTTATGGCCTCTACGCCAAGGGAAACTAAGG
CTCAGAGACATTTAGAAACTTGCTCAAGAATACATAGGGATAAAACCAAT
TTTTATCTTACCTAGGGATAAAGCCAATTTCTTAACTCCCTGTACTGTGT
TTATTTCATCTTATTGTCCCAGATTCTAGCACACATCCCAGCAGCCAGCC
CCTTTCCTGAGAGGAAGTAGGTGTAAGGTCCCAGCCACCTACAGAGGTCA
GAGAGCTCATATGTCTGAAGGTGGCTAGGGATGGAGGAAATCTTCCCATG
CCATGTTCTTCCATCCCTGAAAAATGTGTGTTCTGTCTTTTCAAGCTGCC
ATCAGACTTGATTTCAGTTTGGACTTTGAGTCAGACACATGCAGGTTCGG
ATCCCAGCTCTACCCCCAAACCAGCTACTTAGGTATGGTCTTATGTAGTC
TCCTGCTCACAGTAACCCCTGTGAGCAGGAGACCACATACATCACAACCT
TGGCACAGGGCCTGGCCCTGTCAGTACATGGGTGTTTGACATTTCTGGTG
CTAGGACTTGGATCTCTACGGTAGAGCCTGTAAATCAGAGAGAGGCCCAA
CACTCCACGGGGGAGAGGTGGTGGTGGGCTCTGGTGGGACTTAGGCTCAC
CTAGCTCAGGGCAGCACAAGTGAGAATTAAACTGAATCCCATCAGTGTTA
AGTGAGCAAGATCGGTGACGTCAGCGATGGGTTGTGGTTGGTGGGGATGG
GCAGAGACTCAGCTCCACTGTTGCAGGCCTAGAGCCTAGAGCAGAAAGAA
GGGCAATGGGAATGGGACAAAGAAGAAATGGGAGAGACGTTGTTAAGCTT
TGGATATATTTATTTACAAAAACCTTGAATTTAAAAACTTTGTTGCTTTA
TTTCAAAAGTATTTCTTTTAACACAAAAGAAATGTGTTCATTCCAGACAA
ATTACAAAATAAATGAGGCAAATAAAAAAAGATCAGCACAATCTCATCAC
CCAGAGAGAGCCATCATCTTCCAGAGGTTTTCTCTGCATATTCAAGTAGT
CCACAAACGTTTTATAAATTAGGGAACATATTCTTCTCCAAGCTACTAGT
TACTATTTTTTTATTCTAACAAGCTGTTCCAGCTGTCTTTCTGTGTAAAA
AAAAGGCTATATTTTCACCCCAATTCTAAAAGGTTGCATATTATATCACC
ATGTGGTCGGGCCACTCTATTTGAATTCTGCATTGAAAGGCAGGATAATG
AACATGGAGTGGGAAGAGGGAGGTACAGAGGGACCCTGACTACAGTCCCT
GGCTGCTTGCCAGGATGCTTCCTTCCACGGACACAACTATTGAAGGTCTA
GATGACCCTGACCAAACAGACGCCTTGGCTTCCTTTCCTTTTCCTGGGAC
AGGCTTCCCTGACTGATGGGGGAGCTCCCATGACAGCAACTTTCTAATGT
GTCCTGCATAGACACCACTGGAGAGGGCAGACAACAGTGGGGCCAGGCTG
GACGAGGCGTGTGCGACTGGGGAGAGGGGAAAGGGCAATGAAGAGCACAG
ACCCCTCTCCCCAGCCCCACCATCCAGGGCAGCCCTGAGCCCAACAGCAC
CGGCAGAGTCCAGTGTGAGGAGCAGGGCTAGTCTACGGCGCTCGGTTTTC
CTCTCTGTGGGACTTAGGCTCACCTAGCTCAGGGCAGCACAAGTGAGAAT
TAAACGGAATCCCATTAGTGTTAAGCGAGTGAGATTGGTGACGTCAGTGG
TTTCCATTAGCCTGAATGAGACAGGGAAAGGGGATCTCATGGAAAGGAGC
CCTCGAGAACCCCTAAGGCCTTGGAGAGCTGCCAATGCTGATTGCTTAGT
GTGGGCCAAATTAAAAACAGAGGCATGGAAGCGGCAGCCCAGCCTGGCAA
GGGCCACAGCCACAGGAACCATAGTGCCCACAGGCCTCACCCAGGGTGTC
TGGGACCCTCACAGAAGGGCTTCGAGAAGCAGCCTCAGCTTCCCCATCTG
TAATATGAGAGGCTGAACGAGAGGGTCTTAAAGCCCCTCCAGCATCAGAA
GTTCTGGGAGCTTCCCCGGAACGGTAAGGATATTAAATGTCTTTGGCAGT
AGGTCTTCCAGTGTGACTTGGAGCTGGGCTCACTAAGAAGCAAAGGCCCT
GGGAGTCACAACCAGGGACTCAACCCACGTTGTAGGTGACACCAACCCGG
TGCAACAGCATGACCTCTGTCCTGCTGAAGAGAACCCTGGGGGCCCAGAG
CCCCAGGGCTGAGCACAGGGTGCTGGAGGCCGACGGGCCTGGGCTTTGAC
CCTGGACCCCCTCCCCCTTTTCCAGCTGGGTGCCCCTTTCCTCCACAGAT
GAAGGTACTGACTTCTCCGTGCTCAGCTCCCCACCAGTGCCCATGGAGGA
GAAGGCATGGACACTCACGAGAGGAAGATTCCTCTCCACCTGAGAAATAG
CAGCTCCTGGGGCCGGGGAGCCCACATTCCACCTCTCTTTCACACCTTTG
TTTCTAGAGTCCCTCCTGCTGGCCAGACAGAAGGACGCGGTCCTCAGGAA
CCATGTCCCATTTGCTAGCACAGCCTCTGGCCAAAGTTGGTCCTTCCCCA
AGGGGCCTGGGAGAAAGGCCGGGTTTGGAGGCCAGCCGGTGGAACCAGGA
GACCAGGGTGGTGGAGACCAGCAAGCACTCTCGTTGCAGGGAACAACAGC
CGCTTGCAGGCCGAGCTCTGCCAGAAGCATTGGCAGGACCTGCCTCCCAG
TAAGTGCCACAGCACCACCCAGAACCCCCAGAAGGAGACCAGCAGCATGT
GGGGCCTGATGGTGGTTGCCCAGCTGCTGGCTGGCATCGGGACAGTGCCT
ATTCAGCCATTTGGGATCTCCTATGTGGATGACTTCTCAGAGCCCAGCAA
CTCGCCCCTGTACATCTGTGAGTCACCGGCTTGAAGAGGGCTTTGGGGAG
GGGTGAGGTCCTGATGTAGCACTTGATGAGGCCCTGGGGCACTAAACTGA
GGTCCCACCCTCTTTGCTTACAACCCAGCAAATGAGTTCAGAGGCCTGGG
GATCCGACAGACTTGGTTTGAATGTCAGCTCCATTACCTCCCAGCTGTGT
GATCTGAGGCAAATGGCTGAACCCCAGGGCCCCAGGTTTCTCACCTCCAG
GAGGAGGCTGATATTATCTGTGCGCTGGATGGCTGTGGGATACATGAGAT
GGCATATGTAACCACTTAGCTGGTGTTGGGCACTGGATGGACCCTAAACC
AACAGCACCTACTGTTGCTCTCTGACTAGTCCTTCAGGCACAGGCCAGGA
CACACATATCTTCTCTGAGACATCCACTTAGGAGCAGGTTGGCCTCAAAG
AAGCCCATGCAAAGCAGATAGCAGATGACCACGTGAATGCCCAAATCCTT
CCCTTCGAACAAGCCACTTTGGGACGGGCGTGGTGGCTCATGCCTGTAAT
CCCAGCACTTTGGGAGGCCAAGGCAGGCGGATCACCTGAGGCCGGGAGTT
CGAGACCAGCCTGATCAACATGGAGAAACCCCGTCTCTATTAAAAATAAA
AAATTAGCTGGGCATGGTGGCGCATACCTGTGGTCCCAGCTACTTGGGAT
GCTGAGGCAGGAGAATCACTCGAACCCAGGAGGCAGAGGTTGCAGTGAGC
CAAGATTGCACCATTGCTCTCCAGCATGGGCAACAAGAGTGAAACTCCAT
CTAAAAAAAAAAAAAAAAAAAAAAAAAAAGAACACACCACTTTGAGCATC
CTCAGGGCCTATACTCCAGACCACACAGACCACCCCCACAGGCTCTGTGG
CACCATGAGCTCCTCCGAGGGCCCCCAGCATCGTACCCACAGGGAGCATC
ACCTGGCATTTTTAAATGCCTTGGAGCCTGTTTCCTTCCTGGCAAGCAGG
TCTCTTTGGAAGTTGGCTGGAGGAAGAAATAGCCTTGGTTCTATATGTCT
TGGAATCTTGAGTGGTTCTGGCCTTGCTGGTGGGAGATGCCACAGGTGTG
GGCTTATCAGGCTTGGGAACAGATGAAGTGATATCTCAAGACTCCCCCTC
AGCTTGGGAGCCCAAGGCTGCCAAACTCTCTCCATTTATCTCAGTCTCCT
TCCCCTTACTTCCTTTTCCAGCCATCTTATTTGCCATCTCTGTATTTGGA
CCGGCTTTCGGGTACCTGCTGGGCTCTGTCATGCTGCAGATCTTTGTGGA
CTATGGCAGGGTCAACACAGGTGAGTATGTGCAGAGGACACCCATTCCCA
TCCCCAGCTCAATAAAGCAGCCCCCAGCCCCCTCATCCAATCAGAACAGA
GAATGAAGCTTCAGCTCCAGATGCGCTCTGCCACCTCCTCACTCGCTCAC
TCATCCCTGCAGTCCCTCGGAGAGCCAGGAGCAGACAGTGTGTGCCTCCT
CTCTCAGGGCCCACGCTGGCCACTGTGAGGAACAAGCTGCTGAAGCAAAC
TCACTTTCTGCCCTTAAGAAGTTTGGAGACCCAGGGCAGGACCTCAGGTG
TTGCCACCACAGCGCCCATCTTGGTGGCATTAAAAATATTTCTATGGGCT
GGGTGCGGTGGCTCAAGCCTGTAATCCCAGCACTTTGGGAGGCTGAGGCG
GGTGGATCACGAGGTCAGGAGATCAAGACCACCCTGGCTAACACGGTGAA
ACCCCGTCTCTACTAAAAATACAAAAAAATTAGCCGGGCGTGGTGGCAGA
TGCCTGTAGTCCCAGCTACTTAGGAGGCTGAGGCAGAAGAATGGCGTGAA
CCTGGGAGGCAGAGCTTGCAGTGAGCCGAGATCATGCCACTGCACTCCAG
CCTGGGCGACAGAGTGAGACTCTGTCTGAAAGAAAAAAAAAAAATTCTAT
GGTTCTTTGATTCTAAGATGTTGCTGGTTCTTTAAGATTTAATAAGAGCT
CTTTTGAGGAAAAAAAAGAAACATCATATAAAATATACACACGGATGATG
ACTCACATCCAGAGGGTATAACATGGTGTTTGACTGTGTGGGCGCTGGGA
CGGGACTGCCTGGGTCTCATCCTGGCTCCATCTCTTACTAGCCAAGTGAT
CTTAGGCAAGTAACTTTGTCTTTCTGTGCCTCAGCTTCCTCATCTGTAAA
ATGGAGCTAATGATAGTAAAATAAAGTTGTTTTAAAGATCAGAACAAGCC
TAATGTATATTAAGAAGTCAACAGTATGAGTTCGTATTCTTCAACAAGTG
GTATGGATGAGAATATATAGTCCCTAGACACCAGCATAAGTTCTGAGTTA
ACCATTCAGGGAGCCAGCCCCAGGATGAATGGAGCAAGGGCTTCCTGCCT
TTGAGAGTGTGTCCCACACTACCCAGTCTCCCACAAGATGGGGAAGGTCA
GCACCACAACAGAGGGACAGGCAAAGTGCTGTAGGGTGGAGAATGTGGGG
AGTGTGTGGGTGGGATACGAAGTTCCAGCTCGGCTGAAGGGGTGGCTGAA
AGGCAGGACTGAGCCAGACTCCAGGAAGAGCACTCCCACCACTGCCCTGC
AAACAAGTCACGTCACAGATATCTGGCAGGTGCTAGTTGCAACCATGCTG
CTAGAGCCTAGACTCCCGTCCCTAAGACTGTCAGACCACAGTGCCATCTG
CATGGTGTCATTTGGCTGTACTTCCAATGAAAGTAGAAAGCCAGGGGGTG
TCTTATGCCTCAGGATCAAGCCATCCCTGTCCTTTTGCTGGCACTCCCCG
AATTGGTTCCTGGCTCTTGGTTGCCCAAAGAGACCAGCAACTGAGTGCCC
AGTCCACTCAGGGCAGTGCCCAGGGAAGCTTGTCATGATTTGACTGAACC
GTTCTGCCACCTTCTCCTTCCCTTTATGCCCGGATAGTTTCTTTACACAG
AGAAACACAATGGCAGACTTTTACACGTGTGCAAGAGAATTGCCAAGAGA
AAGCTGCTCCTTTGGAAGCCAGGCAGTTCACACAGTTGATGGTTGTTTCT
TTAGGCTTCTAAGGATTATGGGGCCTCCTTTGTTAGGGATCCATAAAGTT
GGGAGGCAGTAGTTTAATGGTTAAGAGGGCAGGTTTGCATACCAGGGAGA
CACAGGCTTGAAATTGACCAATGAAATGGCACTTACTAGCCTTTACCTGA
GGCAAGGGACTTAACCCCTTCTATCCTCAGTTTCTTGACTGGTGAGAGGT
GGGCAGACTTGCTAGAGAACCCAGTGGAAAACAGATGGAAAGCACGTGGT
CTAGAGTCTGGCCCATGGTGTGCCCTTGTGAGTGAGAGTCAGCTCCCTTC
TTCACTACTTCTGCCAAGGGCTGGGAGTTGAATAAGGAGGTGAAGGTACT
GCTCCCTCCTGCCCGGGGATTTTCTGATGCTGTGCTTCATGTGCAGTGGC
CTTGGTCCTCTGGGAAGACCAATAGCTGGGAGAGGGAATGTTGTTTTGAC
CAAGAATCTAGCTCATGGCTTGCCTCTTCTGCATCCTGGGAAGAAAATCA
GTGAAACTCCCCCTGATCAGAGGTTCTGTTTCCAGTAAGGTGAGATCATG
CATTCAACAAACATTTGCACTCTTCGTTTTCTCTTCAGCTGCAGTTAACT
TGGTCCCGGGTGACCCCCGATGGATTGGAGCCTGGTGGCTAGGCCTGCTC
ATTTCTTCAGCTTTATTGGTTCTCACCTCTTTCCCCTTTTTTTTCTTCCC
TCGAGCAATGCCCATAGGAGCAAAGGTAAGGTTCCAAGAGCCCATGACTC
TACCTGATGGAGTTGAAGAAAGGGATGTGGGGGTAGAAAATAAATAGAGC
TAAAAAGCTGCAGCATGTTAAGCAAATTCAGGGTGAAATCTAAACAAATT
TCATGATGCAGGACTTCCCAGAGGCTTTAAGAGGTGAGGGTGTTGGTGAA
TTTCCAAAAGGTGGTATCTCCTATGTGTTCCCGTACCATGTTAGCATGTC
AAGGATCAGGAGACCTCCAGGGAACAGTTTGGGAAACACTGAGCTTATCT
AAATTGAGCTAAAGAATTAAGAGCCAATAAAGGCCAAGTCTGCGACCTGG
TACAGTCAACTTTTACATTTAATAGGGCCTATCAGTGTTCAGTGTTTGAA
CCTGAAAAACTGAGTAGGAAGGGATTCCTAGCATTGAACAGTGTTGAGGG
TGGCTGAAATTGGAGCATATTTTGAGCAGAGGACCTTCCAAGCATCTTCC
AGTCACGCCTGCTGGGGAAAGGAATCAGGGAAGGATGAGAACGGAGAGCA
AGGAAGAGAAGTCTGGGTAGGGGCTTGATGTTCAGCTGAAGGGTCTAAAG
ATTGTTCTAATTCCATGAAGTTCTAGAGCACTGCAGTGACTGGGTGGCCA
CTTGGGTGGCAATATGGCAGCTAGGTTGAGTTTAGGGGGTAGGGAAGGAA
TGGAGGCTGGAGATCTGAAGAGGCCAGTGCAGAGACCAGGTGAGAGATGG
GGAATGCAGCAATGGTTGTCGGAGAGCTGGGACAGGGCAAAGATAAGGAT
AGACATTCCTAGGGTGGATGCCCAGAAGGACTCAGCAGTGAGCTGGCTTG
GAGGAGAAGAAGGAGCTAAAGTTTATGCCTAGGCTTCAAGTCCCAAAATA
GAGCTCAATGTTTGACAATGAGGTATATAAAATCTAGGCCCAGCCGGGGG
GAGAGGGGCAGGGGTGGGAGGACCTCAGCTTGTCTTGGGGAAGAATTTCA
GACGTTGGCTACACATCATGCAGGTGCTGGGGTCAGGCAGACCTGGGTTT
GAATGAAGCTCTGCTACTTATAAACCAGCTGAGTTGGCCAGGTGTATAAT
CTCACACTTTAGGAGGCCAAGGCAGGAGGATTGCTTGAGCCCAGGTTGAG
ATTGGTGGGTAGGGAAGGAATGAAGTTCAAGACCACCCTGGGCAACATAG
CGAGACCCCATCTCTAAAAAAAAAATTATTTTAAAAACTTAGCTCAGCAT
GGTGTTGAGTGCATGTAGTCCCACCTACTTGGGAGGCTGAGGTGGGAGAA
TCACTTGAGCCCAGGAGTTCGAGGCTGCAGTGAGCTATGACTGCACCACT
GCACACCAGCTTGGGTGACAGAGTGAGACCCTGTCTCTAAAAAAATAAAA
ATAAAAAATAAAAAACAATGAACTAGCTGAGTGAGCTTCGCCAAGTTTCT
TTGTTCCTAAGCTTCCTCTTCTTTACAGTGCTCCAGCTAATTAGAGTAGC
AGTATGGGAAAGCGTTGTGAGGAATGAGACAAAGCATATGAATACTGGGC
ACAGGTCCAGCAGGGAGTAAGCACTCAGTGAATGGGAGAGCCTATTCCTG
GAGATGAGTTCCCAGCAGGATCCTAATGCTGTCAGAGCCCAGGGGCCTTG
CATCTGAAAGGTAGTTCAGAGCTGCAGGGCCTTAGGCTGGAGCTGACAAG
GTCAGAAGAGGTAGGAAGGTTCCCTGGGGTAGTATACCCAAGAAAGTGCA
GGTAAAGGTTTAGGACAGAGCCTTGAGCAGCACTCACCTTTAGGCTCAGG
AGGAAGCAAGAATCCCAGAGAGGAGATAGAAAGGGGGAGGACGGTGGGGC
TGGAAACAGATGGCATTCTAGACCAGAGTGTCCCAGAAGCCAGTATCCCC
TGGAATCTGAGGGTAGGGGAGAAGCGTAGCCATGACAGATGGGGGTGAGG
GGTGGGGTCCAGGAAGACACCTTTGGCAACAGTCATAGGAAAGTTAGATA
TGAAATGATATGCCCACAGGTATCGTGGGAGGCATTGGCATAGATGTGTT
CTGATTGTCGGCCAAAACACCAGGCTCACGGAAATGCAGGTGCTGTTTGC
TTCAGGAGAGCATTCTCTGGATGCCCTCCCATGCACACACGCCTGGAGGG
GGCTCACCTCCACCCAGCCCTGAGCTTCTGAGGCCAGAGACTCAAGGGCT
CAGGGAAGGGTGGCTACAGTGAGCCTGGGCCGTGAACACTCACCCCTTTT
ATTGCCTTCAGAGGGCTCCTGCCACAGCAGATGAAGCAAGGAAGTTGGAG
GAGGCCAAGTCAAGAGGCTCCCTGGTGGATTTCATTAAACGTAACTGACC
CTACAGCCCTGGGTGGGCTGGCACAGTGCGGGGGAGGGGATAGTGGCCAG
GGGGACCCCAGCCCCTGTGGTGTTGTGTGCGGTAAGGGACAGTAGGAGCA
GAGGGCCTGTAACTGGCCCATCCCCTAGGTGTGCCTGCAGCCCAGGGGTT
CTGCCAGAGCCTTGACAGCGGTCTGTTCACAGGGTTTCCATGCATCTTTC
TGAGGCTCCTGATGAACTCACTCTTCGTCCTGGTGGTCCTGGCCCAGTGC
ACCTTCTCCTCCGTCATTGCTGGCCTCTCCACCTTCCTCAACAAGTTCCT
GGAGAAGCAGTATGGCACCTCAGCAGCCTATGCCAACTTCCTCATTGGTG
AGCCCAGAGGGTGCTGCAGGATCCGCAGGGCTTGCCTGGGCCAGGAGGCA
GGCACTCCAGCCACCCATCCAGGGATGGGCGCAGGCCAGACCGGACATAG
GCAGGGTCCGGAGGAGGACTGCCCAGGAGAAAGAATTTTAAGGACAGTTT
TCCTGGTTGGCAATCTAGAAAGTTCTTGGCAGATTTCTGACTCCTGTCCT
TCCAGTCAGGAGTGGGTGGAGGCTGGGGGCTGCCTTCCTCCCTGTAAAGG
GGCTGGAAGTCTGACAGCTTCCCAAAGGCCCTGACATCATCAGGAACAAC
ACAGGAAAGAGGGAAGGTTGAATTTGAGGTTAGACAAAATGCCTCCGGGA
TTTTCTCTTCTTCCCTAGGGACCTGAGTATATTTGCTTTGGTTAGACAAT
ATATGGTCCAGAGATTCTCAACCCAGGCTGCACCAGAGGCTCCTCTGGAG
AGCTTTACAAAACACAGATGCCCAGCCCTATCCCAGGTTAACGAAACCAG
AATCTCTGGAGGTGGCACCCAGCAGTAGTATTTTTTCAACTGTGTTGAGA
ACTACAGATGTGGTCCCACCAAGTACCCTAGGAAATCACTGGGCCCAACC
CAACCCACAGGTGAGTTTAACCATATGGAGTTGGCAAGGTGATCAGTGCA
TGAGATTGGAGGGTGACTCCCGTGCACCCCCTGGCTGGTCCTTGGCTGTG
GCTTTACCCCTCTGGCCTTCCATCTCTGACTGGTACATAGGGGTCATCAC
TCTTGTGTTGCTAACCCCATGCGGTCCCATGCAAAGAGGCCCCAAGGAAT
GAAATGTGCTTAATAAATGCATATTCAACCCACATCTCTTAAGCTGCAAG
AACCCCACTTCTGTATTTTTTGTGTAGGCAAGGCAATCCTGTCTCAAGAT
ATTTCTCTAGATGTGCCTGGCAAGCAGTAAATGCTCAATTAATGTCAGCT
ACTTCGATTACAATTTAACATACCAAATGTTCCTTCCTGAAGCCATGCTT
CCTTCTCAGTGATCCAGCTCAGTGTTTGTAGCCCACATGCAGTCCCAGGC
CTGTGCACCTGGATCACCTCCTTCTTTCCACTTATAGGTGCTGTGAACCT
CCCTGCTGCAGCCTTGGGGATGCTGTTTGGAGGAATCCTCATGAAGCGCT
TTGTTTTCTCTCTACAAGCCATTCCCCGCATAGCTACCACCATCATCACC
ATCTCCATGATCCTTTGTGTTCCTTTGTTCTTCATGGGATGCTCCACCCC
AACTGTGGCCGAAGTCTACCCCCCTAGGTAATGGGAATAAGGGGTAGAGA
GATCCCCTTTAATAAGGCCAGGGCTTAGATCTGGCTGTTATACATCTTCC
TTCCTGCTTCCTTAACTCTATTCTTTGCTGTACACTTTTTTTTTTTTTTG
AGACAGAGTCTCGCTCCGTCACCCAGGCTGTAGTGCAGTGGCGTGATCTT
GGCTCACTGCAAACTCCACCTCCCAGGTTCAAGTGATTCTCCCAGGTTCA
AGCAATTCTCATGCCTCAGCCTCTCGAGTAGCTGGGATTACAGGTGTGTA
CCACCACACCCGGCTAATTTTTGTATTTTTAGTTGAGGCGGGGTTTCACC
ATTTTGGCCAGGCTGGTCATAAACTCCTGACCTCAAGTGATCTGCCTGCC
ACAGCCTCCCAAAATGCTGAAATTACAGGTGTGAGCCACCCCGCCCGGCC
CACTTTTGATCTGATCTCTCACGTGGCAACTTAGAAAATCAGGTCTTGAA
TGGTCTGCTCCATTATAAGAAGAGATCAAAGGCTCTTACTGGGCCACCAG
CAATCAGGGTTAGAGTTCTGTTTTCTTAAGAAGCACCAAAAACTAATTTG
TTTGATTTCAGGATGAATTTGTAGGGCTGTGAGCCACAGGAACCAGGAAA
TAAGCTATGTTTAGCATCAGCTCTGTGCTGGCTAGAATTTCAGTTTCATT
GAGCTGCTGAAGAGAGATGAAAGGAGACTTGAAAAGATCTGGAGAAGAAT
AATGAGAGTGATGGCAGACTTGAAAAATTAGGGCAGGTGAGGAGAAGGTC
AGGTAAAGTCTGCCTGTTGGTTTGCAGACAGCAATATGGGTCACAGTGTT
AACTTTGGCTTTATGTTCAGGAAGAGCTTCAGAGCCCTGTGAGGGGAAGG
ATGCCAACCAAGGGGCATTTTATTTTATTTTTCCTGGGTGCCACCTGTCA
ACAAGCTTTTTTTTAAGACTTTGCCTAATGAAGTTGCAACATTTGGAGAT
GCTGTACCTTTCAGCCCTGGTATTTTCTCTGTTTTTTTTTTTTTTTCTTT
GAAATGACCTCCTCTGTCTTTGGTGGGTGAAGGAGAGAAGGGGTTTGGGG
GAAGGGAGGGTTGCTCGGGGTAGGGAGCTCACCCCTAGTCTGTACGGCAC
TTCTGCAGGCTGACTGTGGATTGAGGAAAGCCGAGAAGCCTTTGGGCCTT
TTGGCAGCGTCCACTGGGAGTTTAGCACATGGCTTTGTCCTCAGACAGGC
CTGGACTGTATACCAGTGTTCCTTACTAGTTGGGGGAAGTTGCTTACCTT
ATCTAAGCTTTGGTTTTCTAATCCACAACAGGCAAATAATTCACTGTAGA
CTCGTTGTGATGATTGTGCATGTAAAGCCCCCGGGCACAGTGCCTGGCAC
GAGATGAACTCTCATTCGTATAATTATACTAGTGAAATATCTGGACAGTG
GTTTGTCAGCCCCTTTCCCAACCCTCCCCGAAGAAGGTCATGAATATCAG
ATACCGGGGAACAGTGGGAACAGTGAGATCAACCAGTGAAATCCCTCTGG
TTGAAGCTCAAGGGGATGGAGTAACTCTGGGCAAGGTCTACATTAAGCCA
GGAGACAGACAGCAAACTAAATCCTGGATTGCCTCAGCGGTCCCAGCCAC
TGTTTTTCGGCTGAAGAAGAGGGAAGGCGAATATTGTCTGTGACCTGCCC
ATTGCAGCTGCCCTTTCTGGGTGGTTGGTTGTCTTGACGCCTACTGTGGC
AAGGGATTCACCAGCCAGGCCTTCCCAGAGCATAATTAGTGGATTTGGGC
TCCCAGTGGCAGAAGAAATGGAGAGATGCCGTGACCACTAAACAGTGGCA
AATTCACTGTTGGCTCCACCAGGCATAATTCAGATCCCAGGGTGGCACTG
GCCCAAAAGAAATAAAGATGCACAGACACACTCACCAGGCCAGGAACTCA
CACTGGGCTGGAGTGTAGGGTTTTTCTTTGGTCTTGCTTTGTTTGATTCC
CCGTTTCCTCTTTTGCTGGCAGCACATCAAGTTCTATACATCCGCAGTCT
CCTGCCTGCCGCAGGGACTGCTCGTGCCCAGATTCTATCTTCCACCCGGT
CTGTGGAGACAATGGAATCGAGTACCTCTCCCCTTGCCATGCCGGCTGCA
GCAACATCAACATGAGCTCTGCAACCTCCAAGCAACTGGTAAGGGCAGCC
AGGGTCCAGAGGCAAGAGGAGCCCCACAGGATCTCGGAGCTCAGCGCATG
CCCTCAAAGACTGTTTCTCGGCTCCAGGATAGGCAGGGCTCCACACACAC
ACTCATTACCAGCATCCTGGCCTCCTCTGAGCCACATCCAACCTCTCTCT
ACCTCCCTACCCTGGGGTCATGACCAGATGCTGACAGCCCACAGTCAATT
AAAGACTGGCCGATGTCCAACAAATTCACATTCTTGAGGCCTAACTGCTC
AGAGCAGAAACCAGTCAAAGCACTCCTTCAGCTGAGGCCTTTGAGCCCCA
TCCTGAGAGATCCTCATGGGCATCTCTGGGTCCTGCAGGCTGGCCGTGGC
CCAGATAAATCGAGGGCATTAGGCTTCTGTGACATGGACCTGGAAGGGTA
GAGCCAGAAGCAGTAGCTGAGGGCACTTGGTTCAACCCCTCAGCTCCCAG
GTGTGGCCACATGGCTGGCCTGAAATCATGCAGCTATTTCGGGCTGACCT
GGCACTTGAACCCAGGGCTCTGCCTCAGTCTGGGGCACTTCTATTCAAAG
CCATGTATCTTCTGGCCACCAGCTTTCCACTTGGGCAACCTCCATTCACT
AGACTCACTTGGCAAAGTACAGTCCTTTTTTCTGTGGCTCCACACCTCCT
AGGATCAGCCAGCAGCAAACTGACTTGAGTCATTTGTTTAGTTATTCAAT
AAACAGAACACTGGGCACAGCCGTGAGTGCTGAGGATGTGATGGTGAACC
CATCAGACAAGGTCCCTGTTCTCATGGAGGGTGCATTCTATCAAATCAAC
ACACAGGATAATTCTAGAAAGTAACCCATGCCATGGAAGGACTTAATAGG
ATGGAGAGTTCCCAGGGGCCTCCGGGAGAGGGTGACATTAGAAGTGAACA
ACCAGAAGAACAGAATTCTATGCACACCAGGAGCAATGCATTCCAGATAA
AGGGAGCAGCAAGTACAAGTCCCAAGGGCATGAATGTACTGGGCATGCTG
TAGGAAAGAAGAGAAAAGGCCGGGCTTGGTGCTGTAGGAGGAGTGTCACA
CCCAGCTGGCCAGGGAGCAGGCAGGAGCCAGAGCCAGGCAGGCCAGGCTT
AGGAACTGCTGAGCAATGAGAAGACTGTGCAGTTAGTGTAAGCTAGGAAC
ATGCCTTATTCCCTTAAGCCCATATGGAGGGGATGGGCAAGCCTAGTGCC
TACTTACCAACCTCCTCTGAAAGGACAGGGCAAGGCTGCTGCCACTGCCA
CCTCTGCGGGCGAAGGAGCGTGGCTCCCAGGCAGAGGAAGGACAAGGAAC
TTGGACAAGATGCTCTTTAAGTTGCCTTCCAGTTGTGGTGGCTTCTGTGG
TTTTCTTATCTTTGTTAACTTACCTCTGAGTCACCTACTTCCAGAAATAA
TCTTGGCAAGTTTCCCGTAACAGCATACATACCTCAAGACAAAAACCCAT
TAGCAAAATATCAAGAAAAAAAATCAGTAAATTATAAAGAAAAAAAATCT
CTAAAAAATCCAGACTGAGGAAAGTAACAGCAATATTTTATATAACTAAC
TTTGGAGTTTCCTAGGGGCCAAGTGAAGAGGGTGCATAGTTACCACTGTC
GGCAGAAGGAAACATACTAGTCCCTTAGTTCCAATATTAGTCTTGTAACT
GCTTGGACACTGAAATTGTCACAGGCTGGGCCATCTGACAAGGGCAGCGA
AGGAAGGTGAAGACCACCAGACAAGGGCAGTGGCCACCAAAGTGAGTTGG
ACAACAGTATTGAGCCATATGGACTTGCAGACTTGAGACAGGGGCCTCCT
TCTCTGGAGTCCCTTCTAGGCAGGAGTTCCTTTCAGGCAGGGAACTCCAG
CAAAGGCCTGACAGAATCAGAACCCAGGGAATTCCACTGTGTTCCCACTG
CCCTCCTACTGTTTATGCCTCCCTCTGCAATAAACACACAAACAGCAAAA
ACAACAATCTGCAAAACCACCACTGCTGGGCACAAGCCCTCCTGGGCCCC
TGGCTCCAAGCTGGGGGTTGGGGGCCTAACCTGGCAGTGTTCCTGGGCAT
CCAGAGGCAGATATGACTTCTGGAGGAGCCCAGCCCAGCCCAGAAGATGG
TTTCTGTTGCCCAAACAGTGACAGAGGTGGGACCCCTGGGGCAGCTTGAA
ACCCTCACCTGGCCCCAGGCTCACCCAGCTTCCCTGGGAACCAGACCCAG
AGGACACAAGGGTTGAAACCTCAGTCTCCAACAAGAGCTGGTGCAGACGT
CTGAAGTGTGTGAAAGTGAAATAATAACAAATTTCCCCCTTTTTCCCTCC
CCTTCCTCTTGAAGATCTATTTGAACTGCAGCTGTGTGACCGGGGGATCC
GCTTCAGCAAAGACAGGATCGTGCCCTGTCCCCTGTGCCCACTTCCTGCT
CCCGGCCATCTTCCTCATCTCCTTCGTGTCCCTGATAGCCTGCATCTCCC
ACAACCCCCTCTACATGATGGTTCTGCGGTGAGTGGCAAACCTCTGTGAG
TCTGGGGCGTGGCTTCTCCTTCCCCACATCTCCCTTTGACTTTGCGCCTA
GTTGCCTGTTTCTCATCTCTCCCCCCTTTTGTGGCCCTCAGAGTCCTTTA
AATGACAATGTGCAAGGTTCTTTTGCTGAGGGGAAATAATACTAATGGCA
ACAGTGCTGGGAGCCCTGCCCAGGGAGGCTCTGTGCTGAGCTCCTCATTG
CAGGGCCTCATTCAGTCCCACAGCGTCCATGTGACACAGGCCCTGTTGGC
GCCTCCACTCTGCACATTGGAGGTTAAATCACAAGCCTAGGCTGGGCGCG
GTGGCTCGTGCCTGTGACCCCAGCACTTTGGGAGGCCAAGGAGGGCAGAT
CACCTGAGGTCAGGAGTTCAAGACCAGCCTGGCCAACATGGCAAAACTCC
GTCTCTTCTAAAAATACAAAAATTAGCTGGGCGTGGTGGCCAGTGCCTGT
AATCCCAGCTTCTTGGGAGGCTGAGGCAGAAGAATCACTTGAACCCAGGA
GGCGGAGGTTGCAGTGAGCCAAGATTGTGCCACTGCACTCCAGCCTGGGT
GACAGAGTGAGACTCCGTCTCAAAAATAATAATAATTAAAAAAAAAATCA
CTAGCCTAGTATGGCAGAGCCAGTGAATGGCTGAGCCATGTGTTGAAGCC
AGGCTGCCTGGCTCCACACCATGGAAATGCCTGTAGGTTTGCTTATTTTT
AAATCCTAAAAGAAATGAGAAGAGCAAAGAGAATTTAGTTGGATGCATAA
AACCAAGGCAGGAAAACGTGGAAAGTTTCAGAGTCAGAGTGAGGGCTATG
GAAAAGGATGAAAATGAGAAAATCCTGGTATGATTGGCTCCAGGACCAAG
TTCCCAGGTCAGGAAGGGAGCCGCTGGCCAGAGCTGGAGGGGAGGAGCGG
GGTGTCCCGTTACAGCTGGCAGCTGGCAATGAATGCTTCTGAGGTTTTCA
CTGGGAATGACCCGCTCTTCACCTCACTGGCTGCAAGGAGAAAACAACAT
TTGTACCAAATTCACCTAACCCACCCCCACTGCCCTCCCTCCTGTCCTCA
CTCACCTCGCAAGCAGAGAGGGCAAAAACACAGGATTTGGACTCCCGGTG
CCAGGATTAAAGTCCCTGCTCTGCCACATACTGGCTGTGTTAGGGTGTTG
CCTAACCTCATTGAGCCTCAGTCTCCTTATTCATGAAATGATGATGATGA
TATCGAACTCAAAGGGTTGGTGTCAGGATTATATGGTAAATTGCTTTGTA
AATATAAGGTGTTCTACATATTGGTTAATTATCACTATTAATCAAATAAT
AATAATAATAATTCACAGCTAGCTAGGTGTCTGTGACCTATTTTCCCCAG
AAGGCTAGGAGAAAAGGAAGGAGCAGATGCAGCCACTTGTTGGGATCTGG
GATTTTACCCTATTTATAAGTGAGCTATCAAGTTAGTGTGAAAGCTATCA
AATCAAACTACTATTTCAAGGATGCTGGCAGAAGATGTGAGACTCCTGGG
TCAAAGACAAAATACTTTATTACAGCACAGAAAGCAGCACAAAACTTAGG
GTGTGTCAGTTATCCTTGACCCCCAAATCCTACAGGGGTGACACAGAGAG
GCCCAGGTGTACACTTTGCAAGAACACTGAGCATAGAAGAGCCACCACTT
CTATGGTGAGCAGTGAGCACCGCTGCTCCTGGTCTTGGAGGAAACGTTAA
CTCATGCCTCAGAGTTGCTCATTGCAACTACAACCCGGAGAAACGGCACA
GGAAAACAGTAGTCAGGACCTTGCATTCTGAGCTTACTCAGCAAGAAGGT
GCAGGGATGCTTGGAACCCACGGCGTGTTGCCTCCTTTGACACCACTGTC
ACCCCCTTCTCTACCGCTCCCCATCCTGCGCATTCAAAGCACTGACAGCT
TCACCTGAGACTCTTCAGCGGGCTTCCCTTCTTGCACCTTGGGTCAGATG
GACCACAGTGGACCCTCCAGCCCTGACATAAAACTACATGAGGCCATGAT
GAGGAGAGGGCACAGAGTAGGAAAATCTCCCTGGCAAGTCGCTATGCAGC
AGGAAGAAGGGCAATCCCTGTCAAATCACCTCCCAGAGGACTCCTGTCAA
ACCCTAGGTACAAGGAGGTCGTGAAGAACAGAGCATCATTTCAACCACAG
GATGGGCTTCTCTAGAAAATCTCTCCTTTCTAGGTCGAGTTGGGGTAGGG
GGTGGAGAGGAGGCCCCAGGATCCCACAACGATTTAAAGATAAATCCTAC
TGGGAAAGATGACAGAATTGATGCTATTTAGTGTGAAGAAGGAGAGGCTT
AAAGATGACTTTGTAGGCCGGGTGTGGTGGCTCACACCTGTAATCCCAGC
ACTTTGGGAGGCCAAGGTGGGTGGACCACCTGAGGTCAGGCGTTCAAGAC
CAGCCTGGCCAACATGGCGAAACCTTGTCTCTACTAAAATTACAAAAGTT
AGCCGGGCATGGTGATGGGCGCCTGTAGTCCCAGCTACTTGGGAGGCTGA
GGCAGGAGAATTGGTTGAACCTGGGAAGTGGAGGTTGCAGTGAGCCAAGA
CTGAGCCACTGCACTCCAGCCTGGGTGACAGAATGAGACTCTGTCTCCGA
AAAAAAAAAAAAAAAAAAAAGATGACTTTGTAGGTGACCCAAGCATTATT
TATATGAGTCCTGGCCCCCAATTAGCCATGGGAACACAAGCTGGAAGTGA
AGGCTAGCTGAAAACGTGGAAGGAGAAAGAGAGGTCAGAAGGTCAGCTTT
CATTCAGGGCCACATGGGACAGAGACTAGAAGGGGCCCAAGATCTCAATC
AAAAGCCAAATTGGGGTGGCCAAGTCCCGTTTCATTGAGAATCTAGGGGA
GTCTGCCCAGGGCAAGGGCCAAGACAAGGCAGGTATCTATTATGAACTGG
ACCACTGGAGGGATCTGTAGACACAACAGGATTTGAGACACTTTGCATTG
TGGGGGTCCCAAGGGGTGCCTCTTCCCCTGCCCCAATGAGTATTTGTCTC
ACTGGGCCATCCCTCAGGGGGACTGGGCAAAGGTCCCTTTCCAGTTTGCT
AGCAGAACTGTGATGGTGGTGGTGAAACAAACCACAGGTAGAGGCCTGGG
GATTACTTCTGAGCTCCCTTCCTCACTGTCCCCTAGCACTGAAGGCTCTT
CCTGACAAGCACCCACCGGGAGTGTGTGTGCTCAGAGCACAGAGAGTCCT
GGAGTCAAGTCACTCATCAGGTGCCTCTGCTGGCCAGCTGCTGGCTCCCC
ACCTTGTGTGGAGGTGTCCTCCCTCTCTCCACTCCCCAGCTGGGATGGCA
GGGCCGTCCCCTGCTCTCTCTGAATCTCCTTCCTCAGCCAAACACATTTT
CCTCTCTTGAAGGAAGATGGTTAGGGGCAGGACCAGCTACCTAATTTGCA
GGACCTATTGCAAATGAAAATGTAAAGCCCCTGGCTCAGAAATTAAGAAT
TTCCAGACAGCTGCAGCAGAGCATTAAGCCAAACATGGGACCTTTCTGAG
GGTGGAGTTGTATGCACACACCCACAGAGCTAGCCTGGGGTAGGGAATTC
TTTTAGCTGTGCACAGAAGCCTGCAGCCCAAGGGCCCTAATTACACTTTG
CAGCAGGGCTGTCTTTTACTGTTATAAGACATTCTGTGGGAGTTGTTCAG
ACCTGCTGCTATCAACACACCCACAGCATCCTTCTCTCCACTCCTAGCCT
CTACTGCCCTCTGCTTCTTGAAGGGGGCTCTGGGCAGCCCTGAAAATCAC
ACAGACTGGTGGTTCTCAGTGATGCATCAGAATCTCCTGGAGGTAAAATA
CAGATTGCTGGGCCCCATCCCCAGACTTTCCGACTCAGTAAGTCTGGGGT
GGGCCTGAAAATCTGCATATCTAACAAGTTCCCAGGGGATGCTGATGATC
CAGGGACCACGCTTTGAGGCCACTGATGTAAAGAACTAGAGCATTCAGCC
CAGGTGAACCCGGTCACCAGAGGGCTCGTCAGGCCACATCAGGGCTGGTT
CTGACCTGCTCTGAGGCTGGCTGCCCCTTACCCAAGGGATCGTGCAGCCC
TGAATCCTCCCACTGCTGATTTCCTGTCTGTCTTTTCAGTGTGGTGAACC
AGGAGGAAAAGTCATTTGCCATCGGGGTGCAGTTCTTGTTGATGCGCTTG
CTGGGTAAGTGTCTAAGACCCCTCTTCCCAAGAAGTGGCCCCAGGCGATG
GGGCATCTGAAGGCTATGGGTGCAGGGTAGCGATGGAGGAAGCACCATCT
CATCTCCATGGCGAAGAGAAGCTGTGCATAATCAACAGCAGATGCCAAAA
TCCATGTGGACGGTTTCCCAGATTGCTTGAGGTTAGGAAAGACTCCATCT
ATGGCATTCATTCCTAACTAAGTTATGTAAGAAAAATGAAAAGTGAACAC
AGCTGAGAATACCTAATAGAGTGTTAAAGGCAGGTATTTTTAAATCCTTC
TCACAAAACTCACTGTAGGAACAAAACTTTGCATTCCTGCCAAGCCAGAG
CAGCGGCTGAGCCCAGCCCTGGCTTCCTTTCCATTTTACAAAATTCACTC
CTCCTAAAACTATTTAGCAAAAGTTTCTTCCCTGTGGGGTCCTCTGCTGG
TGAGTCCTGCCTGCCCTCAAGCAGTCCAGAGCCCACCAGAGGAATGGAGC
TGTGCACAAGTGATCAGCAGTGCCCCAGGGGAGAGCGAGCTGCTTGAGGC
AGCAGGAGTGCTCTCCCGGGGTGCCTGGGCTGAGGGCTGGGGGCACAATG
CAGGCCCATTCCTGAACTGCTCACACTGGCTGTTCCTTCTTGACATATCT
GTGCACTAGACTTAACACAAGGCAGCAATAGAGAGATCCTTGTAATGACA
GGCACACAGTAGTAAAGGGACCTAATTGAACTGAGGATGTGTTAATTAAG
GGTGCAAACTACAGAGGGGAATATGGAGACTCCAGCAAGAGGAACTTCAG
TTACTATAAGGTGTCGTTTCTGACTATAAATCCTGAAATAATGTATAGCC
CACTGTGCCTCATTACCCAGGGGCCAGGTCAACACCGCGCCCTGCAGTGG
CTTCCATCCCCATTTTGAGATGTATTGGTGATGTCTCTACTGGGGCCAGG
TGAGCGGGAGCCCTTGCTGAGCCCAATCCAGTCCCAGGCTGGGAGAAGTG
TTTGGATGATGGGAATGAACCAGCTTTTTCAGTGGGATTTCTTGTTCCTC
TAGTCCCTGCAGAGCCCCTTCAGACCCTGCTCCTGCTGCCTGCATTATAA
ACACAGAGGAAGCAGGGCGGCAGGATCTACCTGTCCTCCCAGCAGAGGCT
CTGCTGTCATCAGAGCTCCTTATTTTCATTTATAACTTAACTTTCTCTAA
CACTTCTGCTGGGAAAGAGACCTCACATTCCTCTCAGCCCATCCAGCACC
TAGGATAGGCTCTAAAGAGCCCTTTCCTGCTTCCTCCAGCAACCTTGAAA
ATGACCTTGAAAACAAGGACTCTCTCGGACTCTCTCGCACTGCTTGAAGC
ACTCGCTGCTTTTGCTTTTCCATTTGAGCTCAGATAACTCAATGTGGGAC
CACAATGAGAAAACCCAGGCCTGGAGGGGTTCCATTTATCACCCAAGAAC
ACAATCCACTCTTGGGTAAAAAAAAGGACTTCCTATTACAGGTTTTAAAT
TACACATTGGGATTGGGTTGACAATGTTTCCGGACTTTGCTTCTACATAA
GGAAGTGTCATATATCACTCTGGCCAGTTTTCATCCCCCTGTTCACCTGG
AGACTTGAGAGTGAGCAGGATCTGAGTAGAGAGGCTTCTTCTTAGAGTTT
CCCACTTGTGGGCAGTGCCACAAGTGGAGCATTGTCTGTCTTTCTTCTGG
AAGCAGCTGCTCACAGGGCCCTAAACAAAGTCTGGGAGGCTGTTGTCAGC
ACTGTGGTGCTGAGAATGGGATGGGCCAGCCTTGGTGTAAATCCTGGACA
AGCCCTCCGCAGTCCAGTAACTGCCTAGAGCTGTAGCTAGTGGTACGCTA
CCCACAGGTAGTGTGGCATCCGGAATATTAGACAGCCAGGAAGGCATAGC
CCCTGCCAAGCCAAATCTTAACACTCAGCTGCTGGGAAGTTCGGGGTCCC
AGAGCAGAAGCTTTGAGCAGCCAGGTATTGGGAGGTCTGCAGAGGGGTGT
TGTCTAATCCAATAGGGCAGCTTGGTGTGGTGGAAGTACATGGGATTCGA
ACCTCCATAAACCAGGGTTCCAAGTCTGGCCTTGACTTCTCTTGGCTGTG
TTACAGTGTCCCAGCCAAGAACCCCTTCCTCTTCACTAAAAAGTGGGCAC
CCACACCTCGGGGCCAGTGTGAGGACCTGAGAAGTCGATGCCTGGAGAGC
CTCCTGCCACACAGTGGAGGCCCGTGTATCTCCACTCTGAGCTGATCTCA
GGAGAACTATGTTGGATGTCTGAGCCGGCATCAAGGTGATCCCTGTGACT
TACTCTGACTTGAAAGAATGGCCCAGGTCCCTTTTGTCTTCTAAGCCTTC
TGGTGAAAGAGAAATTCAGGCTTCACTGAAAGCTATTCTGCTTTCCAGAG
TCACTGTGAGTCCTATGGGTACGGCAGAGCCGTTCTCATGTATGTGAACT
GTGTCGGGGACTTGGCTCCCACACCCCTGCTGGACACCTCTGCCTGCAGT
TCCACCTGACCAGGGCTTTCTGAACCCTCTTCCAGCCTGGCTGCCATCTC
CAGCCCTCTATGGCCTCACCATTGACCACTCCTGCATCCGGTGGAACTCG
CTGTGCTTGGGGAGGCGAGGGGCCTGCGCCTACTATGACAACGATGCTCT
CCGAGACAGGTGAGGGCCCATCATGTATGTGTGTCCCAGAGCCAGGCCCT
GGGCTTGAGATGACAGTAGTCATATGCCCAGTGGCTACTGTGGGGGCTGT
CAGGCTTCCAAGGAAGAGAACATGAAGGGCCACCTCTGCGGCCCTGCCAT
GGACAGGGAGCAGGGGCTACTATAACAGGCATTTCAGGGCTCACAGGGAG
ATGTCCAGGGACTTCCATGACCCGAAAGCCCTTGGAAGGGGCAGCAGACC
CAGGGACTTCCCTGACCCGAAAGCCTTTGGAAGGGGCAGCAGCAGAAAGA
AGGGGCGGGGATGGCTGGAGCCCAAGGAAGAGGCCATGGCTGTGGAAAAA
TTGGGGGCAGCCTCCTGCTGCTCAGAGACAGAGGGGGGCCACATCATGGC
TCCTGGCAGAGGTCCAGGGCAGGGGGACCAACCTGCCTCAATGGGCCAAG
TCCAGCTGCAGTGCTATTGGGTTCTGCACCCCTTCCAGCCACCTCACCTG
CTGCCGTTGTCATATGGCCCAGAAACCACAGGAAGGTGGGCTTGAGGGGA
CTGGGCAGGGGCTGGAAATGGTATGGGAGTGGCAGAGAAGACCACAAAAG
GGACAGGCCAAGAGGGCAGTGAGAAGATGCCTCTCTCCTTCTGGCTCTGC
TTGGAGGTCAGGACCGTCTGGGGGTGGCTGGGGCAGCTGGGTCACCTCTG
GCCCTCTGGAAAGAGCCAGTGGAGGGATGATGAGCCCTGGATTAAGAGTC
CCATGCCTACTCCCTGCCTGCTTCCCTACAGCTTTGTTCCCTTTTGGGTC
AAAGAAATGAGGAAAGTGATCCTCACCTGGATGGTGCCCACACCTTCCGC
GGGCCAGCGTGGGAATGATGTGGTTCTTCCCAGTGGTCCTGGCCCTGGGA
TGTGGGCTGCAGAGCCCCCTCCCTCACCAGTCCTGCCTCCTCCTGCCCAC
AGGTACCTGGGCCTGCAGATGGGCTACAAGGCGCTGGGCATGCTGCTGCT
TTGCTTCATCAGCTGGAGGGTGAAGAAGAACAAGGAGTACAACGTGCAGA
AGGCGGCAGGCCTCATCTGACCCCACCCTGGGCCACTGCCCTGCTCCAGA
GAGTGGACCTTGACTCTTCCACACCTGCCTATACTCACTAATGTTAACAC
GTCATTTCCTTTTTGTATTTTTAAACAAGAAAGAAAACCCCAGTCCTCAT
TTGCCTTCCCTACCTCTTCCTCCCAGAGTCCTCCCCACAGTTCCTAAGGG
CCACTGTGTACCCGGGCTGTGTGGGCCAGAACTGGGGGGCTGAGTCTTCC
CTGGCCCCTTGGAAGAGGCCCCCAGATGCCCAGGCTCACTTCAGTGTTGA
GTCCTCCATTGAGGATGCCCACTGAGGCAGCCAGGCCCCTCACCAGCCCT
GGGGGGAATCCTAAACAGAGAGAGAAAAAGGGTATCTGCCCTTCTTGCCA
GGCAGCTCCACTCTCCCGCTGACTGCCCACACCCTGCAGAGTGGCAGGGG
TGAAAGGAAGAAGGAAGTGGCTGAGTTATTAATAGCCAGAGCCACTGGGA
GACTGGGGAGACTGGCTGTAACCCCCTTCACACCTGGGTTTGGCATCAGC
ACAGACTACGGGAGGGGCTGGCTCCCTCCCCCTCAGACCCTCACTTCCTG
TACCTAGAGGCCATTCTGGATGCTGCCATGTTGGGAAGTACAGTCTCTGC
CCATTACCTGCATGCAGGCACCAGAGCAGGGACTGAGAAACCCCAAGGAT
GGGTCATCTAAGTGCTGTCCATATGAACCCTGGACTTTCTGTCCTTAGAT
CCTCACATGTTATCCCTGTCTTTCTGGGGTACGTTTCAAACTGAGGAAGC
TACAACACAGTGAAGACCCAAGGAAGGCCTATGAAAAGGTCCTGATGCCC
AACCTCCCACCCCTTCAATGTGGGGACGAGACCCCCTCATCTCAGAGTAA
TGGGAAGAACCTCCCACATCTCCCTGGCAGCAGATGAGGTGGCTTCACAT
GCACTTCCCTGTCTGGACTTCAGCCCGTATTCCGAGGAGTAGAGAGGCAG
AAGAGATGTCAGCAAAGCAAGTGATGAAGCAGAGTGGATGTCCACTGTCA
CCAAGCTGGATGGCAAGCTGCGGCCCACAAAACAGCCAGTCAGGTTGGCT
TTCCTGGTTTCAGACATGCTCATACCATTCCCATTTTCTCAGCCTCTTCT
CACTGCCTCCAGAGAGGTGGGTGCCTGGGTTGAGAGACACAGCTGCTACG
TGATAGATGTTGAGAGACAGAAGCCAACGAAGGAGGTCATTCATCAACAA
ATATATTTATTGGAGACCGACTTTGTGCAAAGCAATGCTAATCAGGGTTC
TCCATGGAGCTTCCCTCAGCTCTTACCTCACCTCCCTCCATTTACATTAG
GGCCTTCTCCCAGGGTGTGCTCGGTGGGCAGTGTGGGACTGGGGGTGTGG
GAGTTGGTGAGAGCAGGAGGAGAGGTGGGGACAGCAAGAAGCCACAGATT
GGCATGAAGGATCCTGACCTGACTATCCATGCCATCCATGGCCCCCAGAC
TGACTCTGCACCTGGCCCTTTGCCAGACAGCTCTGTCTCCCCATGTCCTC
TGGAACAGCTGGGCATGGGTCATGGCCATTCATGACCCTTAAGTGCCACC
CTTCTTGGAAGACCCCCTCCAGAAGCATACTGGAAGCCACCTCTGGAAAA
GCCTCATATGGTGATATGCCAAAATATTTATGTCAATGTCCAAACAAAGT
CCAATGCCATGAGACTGAAGTCTTTGTGGAAACCACTGTTACAGACAAGC
TTATTTCCAAAGCCACCTCATTTCCAAACATCTCACTCAGGAAGGGAGGC
TCAATGTAACCTCAGGGGCCAGTTTTAGCATTTGAAATGGTTCTGCTTGG
AAAATGATGCCCTGCAACTAACCCTGGTCTTTCCCATGGCAATTTAACCA
CATTTGGAAGGCACTGCCTTCAGCTGAGTTTATGAACAATGAATGCCAAC
CTTCAGGTTCTAGAAGATTGGTTGCACTCCCAAACCTTTATTCTATTATA
TTACTATTAAAATATTCTAATTTTGCTATTGAGGTAACTTGGTCATGAAT
TTATGTTGTCATATACATCTGTCAGAGAACAGACAGCACCTGCACTTTTT
AATTTTATTTTATTTTTATTTGTATTTTATTTTTGAGATGGAGTCTTGCT
CTGTCACCCAGGCTGGAGTGCAGTGGCACAATCTTGGCTCACTGCAACCT
CTGCCTCCTTGGTTCAAGCGATTCTCCTGCCTCAGCCTCCAGAGTAACTG
GGATTACAGGCCTGCGCCACCACACCTGGCTAATTTTTGTACTTTTAGTA
GAGACAGGGTTTCACCATGTTGGCCAGGCTGGTCTCAAACTCCTGACCTC
AAGTGATCCTCCCGCCTCAGCCTCCCAAAGTGTTGGGATTACAGGCATGA
GCCACTGCGCCCTGCCGGCACCTGCATCTTGTAAACAGAGGAAGTGCTCA
TGACATAGACCCCAGGGGCCCATGGCCATGGAGATTAGAGGTAATGCAAC
TGTCAACCCATTAGCCTGGTATCAGGCAAGTACCAGACACTGCTTATTCC
TTCAGTGAAGTTCTGCTGGGCTCTGAGAAGGCCGCATCACTGCTCTTGGG
TTTATATCCAGAAGCGGAGCTGGACGCAAACAGCTATGTCCATGGCACAA
AACAGAATTAATATCATAATCAAATTGATCACATGGTCACAACCAGTACT
ACTACTCCCAAGAAAATCTTTCTTTTCAGCTGTCAGCAGGGAATATAATA
GTGTCACTTTCATTTCACTTCTAGAGCAAAGCTTAACAAACTTTCTTGAG
TGTGTGACACTCACAGAGGATGCAAATTCCCAACACCCTCCACCCAGCCC
CAGCGTCTACTTCTGTGGGTCTAATGGGGACCCAGGAATGGCTCTTTTAA
CGGCCCCCTCATGAGATTGATGTGGGTAGTTCCCAAATCCTGTGAGGTGG
GCTGTCAGTGCAATAGAGCCTGGGGTATGCCCATTTGTGCTTAGGCCACT
GAGAAATTTACAGGCTCGATAGGCCTTTGGTATGTTCAAAGGTGAGTGAA
GGATAACAAGGTGAAGAGATGCCTGTGAATCTGATTTTTTGGATGGCTGC
AACTAAGAGTCAATGGGCATGAAAAACAATCCAAGAGCTCACATTTATAT
GAGGCCCTTATCAGTACTGAGAGAGACATTCTCACTTCACAAGCTTGGAT
GTGCAAAAAGAAAAAAATCACTTATTTCTTTAAATCAGTTATTGATTTCA
AACCCCCCATCCTACTGTCTGGCTGGAAATTCAATATCAACCATGCTTGG
GTCTGGCTCCTAGAGCCATCTGAAGGTCCTTGGATGCCTACGCATTCAAG
GGTGCCATTGTGGGTCTTTGGTCCCTTTCCCATTTGAGGCTGTTTAATGG
CCCTGCTTCTCACCTGAGCCTGGAAACCCTACAAAGCCGAGACCCCAGGG
TTAGGCCCACCTCCCTGTGCACACTCGCGGCTATGCCCTCTCCCGGCCCC
AGTCCTAGGAAGCCCACTCCTCTGCACCACTCCCTCTCCCTACAGAATCC
CGCTCCTCAAGGGCTGAACAAAAAAGCCGGAAAAGGAAGAGTGTGCAGGA
ACCTTTTTTATTCAATCCTTCTTTTACCCTTCTTGGAAAAAGCGCAAATA
CAGTGATAACTAAATACCGTATCTCTATCAATTATCCTTTGGCCCAAAGC
CCTTTGGGGTGATCCTCCATGATGGATGCCCCATGTAAAGGTGAGGAATC
TGATCCCGAGGAAGCAACCATTGTGGCCAATTTCACATAGATAATAGCTG
GCCAGGTCTAGACTCAAACCAGGCTGTCCACTTCTCTCTGGTCCAGCCCA
ATTGCCCCAGCTGAGTCATTCTGAAGAGTCAGCATGGACTAAAATGGTGG
GGAGGCCCCTCTTTTCCCACTGATGTTCACTGGCCCATGGGAGGGAAGTG
GGGGAATCTCTTGAGGGCCATTAAGGTGGGTAACAAAGGCAAAAGAACCA
GGTAACAGTTCATTTACTTACCTTCTATTCAACTAGCAACAACATCACAA
CTATTCATTAAACAGGGCCAATTTATCCCTATAGCACCATGAGTTAAAGA
GAGTTGTGGAGAAAAGAGACAAAGAGGGAAGGGTTTTGAGAAGAAAAGGC
CCTGAGAATCCTGCACACAGACCTTCAAGGGAACAGCTCCATCTGGTGAC
TGCTTAGCAGATTCTTGCTCAAGAAACAAAGCCATATTGTGATTTTATTC
AACCACTCTCAAAGATGGAGAGATACAGTTAAGCTGGAGGCTGAAGCCAG
TCAGTATCAAATTGATACTAAAATATTATATTGCAGCAAATCCAGAAGAA
ATACATCCTATCTTTCGCAACCACAGGAAGTAAATACCTGGAAGAAAGGT
CCATCTCCTGCCAGTAGCACCAGACACCAGATTCATTATAACCTTTCATC
AGAGATTAAATATGGAAACAGATCCTGGAAAAGCCCTTGGCAAATCATAA
ATGAGAGGAAGTTTTTCTCCATGGCATACCCTGGCCATTCCTCCACTCTC
CAGCCACATCATGTCTCCAGCCCTGCTACAACTTGACAGCAATAATAATA
ACTAAAATGTGCACAGATCTTTACAGTTTACCAAGCCATTAACCCATTTA
AAACACTTCTTTAATCAGTAAATCAGTGGGGAAAGGCTTGGCTATGCTGT
GGTAACAAACCTCTCGAATCTTAGTCACTTGACACAACAAAAGATGGTAT
CTCGCTCACAGAACCTGTGGTGATCTGGGTGACTCTCAGGTCAGTGGCTC
AGTAGTGCCCCTCTGCCTGCTCAGGGCCTGGAGGATTGGCCTGAGAGCCC
CCCCAACCAGTGTCTCAGTAACTCAGACTGTTTGGAACCTGAGGTACCTC
CATTTCATTATAAGCTGCCATGATGGATGGGGCAGGGGAAGAAAGTCCTG
GAAGATCCCGCACTGCTAAGCAATGCTTTGTCCCAAAGTAACGCTCTTCA
CTCTGCTCCCAGCCTAGCTGCCGCAAGAGCGGAACAGAATATTCTGTGTG
CTCAGGAGAAGAAAAGAGCAGATATGGTTGAGCACTAAAACCCTCTACTG
TGGGTAGTGAACCTAGGTGGACACAGTTGCTCACCGGGACACTTAAACCA
GCTTTGCGGGGCTGTCGGGGTTGGGAAGCACAGTCTGGAAAGATTTCCCA
ATGGAGGAAGCACCAATTGTGGGTCCCTGAAGCTGAGGGGATGATGCCCA
GGCAAGGCCGAGGCAGTCTGCAGAGGGCTGGGGAGGTGGGAAAGACGATT
TTGCTCAGAGGAGGCAGAGATGGGGTGGTGGCTGAGAGAGATGCAGGGTC
AAGGAAAGGATGGCTTCAGAGAGGAGAGATTTGCTGGGGCAAAAGGCCAC
AGGATGGGGAGGCTGCGGGGTCCAAGAGGACAGCTGATGGAGCGAGACCC
TGCAGGCAAGGAGGAGATGGGGTGACTCATGGCAGAATCGCACCAGAGTT
CCCACAGCCCTGTGCGGCCACTATGACTAGTCCCATCCTGTACATGGAGA
AAACGAGGCTCCTTCCAGTTGTCCCTCACGCCTCCCTTGATTTATGTACT
ACCTGATGTGTATAAGGACAGGTGGTGACAAACACATGGTCCCTACCCTT
CAAGGACATATAATCCAGAGCTAAGACAAGACCTTAACACAGGGCAAAGT
ACTATGCTCTGAGTTCATTTGATGAGCGATGGGAGAACGGGCTTGATCCT
AGCCATGCGGGAGAGACTGGAATTCAAGAGAGTCAAATGTGCTGGGAAAG
ACTTTCTGGGCAAAAGGAATAAGCAAAGGTGTGCAAAGGTAAGGAAGCAG
GAAACCTTTGCACATCTTGAGAACTCGCCGGCCAGAGGCCTGTGAAGGAG
CGCAGTGGTCAGGCAGGAGAGGCAGTCGGATGCAGGCTGAGACCATCCTG
GAGGGTCTTGAGGCTGGCTTACCCCAGGCTGTGAGTAGTGGGCTCAGCCT
AGTCCCGGCTCCTGGGTTAGTGCTCACGTGTCTCTCTGCCCCCTCAGGGC
CTGGAGGAATGGCCTTAGAGCCCCCACCAACCACGCTCAGGAAGGCATTC
CTAGCTCAGAGTACCCTCCTGGAGTCTACACTGGAAGGGGCTCCTGAGTG
GGCCGCGCCACACCCCGAGGAGCAGAGGCGCAGTCCTCCCGCGTGCTCCC
AGCACACTCCACCCCTACCCAGCACACCCACGGGGCCCCCACCTTGCTCA
CCTGGGGGGAACCACCCACTCTGCGCCCTCTCAGGGAGAGGTGGTGGCCG
CTGCTCTATCCCCTCCCTCTCTTCCTCTTCCACCTTCTCTCTCTTCTCAT
CTGGGTGCTGGAACCCAAGGGTGAAACTGAGAGTCAGAAAGTCTCAGTCT
CAAGGCAGAGCGGGGCAACTAATTTAAAACGGCTTCTACTCCGGCCCCCA
GTGTGACTAATCTCCTTCTCTTCGCTCCACAGGTCTATAAATGGGCCACA
GTAGGCACCCCTTTTTAAATCTGTAAAATCCCCAAATCCATTAAAAGTGA
CTTCAGAGGTCCCAGATAGGGACCAACATTTGTTTAAACCTTTTCCTCAT
TACATCTCATGACCGCCAGCTTACAAAAGAGAGACTGGGGGTTCTGGATG
AGACGTATGCAATCCCTGAGTCCACCTTGGAAAGGACAGAGGCAGGTGTC
ACACCGTTCCTCTTTGACCCGAATCGCTTGAATCCCAGAGTTTCAACAGC
TCAGGTGGGTTGAAGGCTCAGAATGTACCAGACATCCGAAGCGCTTTACA
TACATTTGCTCCTTGAATCTTCACGACAGCCCTGTGAGATGCAATAATCA
TGCCATTCCACAGACGAGGAAACGGGCCCAGGGGACTGCGGTCTCGGCTC
CTTTCCTCTGGCTTTCTCAGAAAAGGCGCGGAGGCCCCCGCCGGGTGGGC
GAGGCGGGGTGGGCGGCCCCTTGCGCCCTGGAGTCGCATTCCGCCACCAG
GGGGCGCCGCCCCTCCGCCGCGGCCCGGGGAAGCCACCCGCGGAGGCGCC
ACGCCCAGCCCGCCCACGGGACGCCCCCGCGGCCCCACAGCCAGCCGGGC
GGGACTCCACCACCCGGCTCAGCGCGCCTCCGATAGCCCGCTTCCCCGCC
GGGGCGCCCTCGGGCCTGCGGGGCTCCCCGCGCCCACCGCACGCACGCAC
GCACGCCTTCCCCTACTCGGAGGGCCCGGCGGCTGAGCCCACCGGCGCCT
GCTCACGGGACACAGCCTCGAGAAAACTTCCTCTCTGACTGCCCGCGATA
AAGCACCGTTTAGTCTATTTATGTTTTCTGCACGAAAGTGTATTAAACAC
GTTAGAGAACAAGAGGAGTAGAGAGTAAGGTGCCACGAGGCACCGAGAGG
GCCCGCGGGAACACACGGCTTCCTCAGGCTCTCTGACCGGCCGTGAGCAG
GGTGGTCCCCGGCCGCATTGTGGGGCCATTCGCACCCAGAAGCTGTCAGC
CTCCCCACTGCGTCACTGGCACCATCTGGGAACCATTATTCAGCCACTGC
AGGGGGCTAGGTAGCCTCTGCTTCCTCCTTCCAATTCCCAAAACCTTCAC
CACAGCACCTCCTGGGGCCTGGCCCCGAGATAGGCTTGCATGTCGGAAAG
AAACTGTTGTCAGCATTCCAGGCACTGTCCACCCCGGGAAAGGGGCAGGT
ACCCATCCCGTCACAGACTCAGGTACCCGGGCAAGCCGGGCACCAGGCCT
ATAGAGGTGAACAAGACAAACAGAAGGCAGACAGACAACCAAGAAAGCAT
CCAAACTGTGGCTGCAATTGGCTGATCAATTACAGGTGCAATAGTGCCAT
GAAGGAGAAGTGCAAACAGACAGACAGATGCCTGCATGGAAATGCAGGGG
GAAGCCGGTGTATGTGGTGGATTAACAAAGGTGAAAGAGGCCCGTTCCCT
AGGGATTGGAGATCTGGAGCAGAGATGAGCTCTAACTGTGCCTGGAAGGA
TAGGGTAAGTTTGTTGACAGGGGCCTTAGAGAAGGTGTTCTGGGCGGAGG
GGACTGCAGGAGTGAAGACTCAGAGATGAGAAAACTGGACCATGTTTGCC
TGGAGGGCAGGGTGTAAGTAGCGCCCCCAGTTGCAAGGTGCCTCAGGCAG
AGCGTGGTGAGTGGTGGATTCTAGAGGGAGAGAGTGTATCATGTACATCT
CCTTTAGTAACTAACAGGGTGCTGCTGGTGGATGATGGGGCGTCGCTGAC
AGAATTTTAACAGAGGCAAGATGCAACCAGATTTCAGGGTGTAAAAGGCC
ACTGGGGCTACATGTGTACTTGGAAGAGGGAACCTGGCTGGCAAAGGTCC
TTAGGAGGGAGGCCTACTTGTCATTATGGCCACTGGGATACTAGAGTAGG
AGACGTCAGAGCCGAGGGCAGGACCAGCAAGCAGCAGGAGCATCCCAGGC
ACCCACAGGCTCCTGGCAGCCAGGATGAGCCAGCGCAGAATGCAGGGTTA
GCAAAAGGCCATGCGGGAGCCCATCCCAGCACCAGAACTTGCCTTGTTAT
AAAACAGGCATTGGAAACTGGCTGAGCCTGGGCCCAGCACAAGAGTTCTA
GTTGGCCTACAAGATATTTGCATGTCTTTAAGTTAGCTCCAAACACTGGG
AGAGTTTGACTGGGGTTTTCTCTTGAAAAACCCTGGTGCAGCATTCCAGC
CCAAAAACAATGGATTGGAGCTGAGTAGAGGGCAGTCCTTCAGATGGACC
CTGTCCTCTCAGGGTGGCTGCGGTTCCCACATCATCTGCCTGGCCCTTGT
AGGTATTTAAAACTTTCACACACACACACACACACACACACACACACACA
CTCAAAAAAGTCAAGTTAAAACACCAGAAATCCAGGTGGATGTTGGAAAA
GACAAGCCCAGAAGGAAAGAAAGGCTGTTTGAGAAATAATGACCAGGGAA
GATTGGCCGTGCTTGAGGATGGATGGGCAGAAGATGTGAGGAACAAGTGG
TGATCGCCAGGTGACTGCTTGTGGCCTGCTAGAGAATCAAATCCAACATT
GAGCTCCTCGTCCTCCCCAAACCTCCTTTCACTGACTTTCCTGTCTCAGT
TAATGGCATTCCAACCTTAGGTGATTAGACCAAAAACTTGGAGACAATAG
AAAATAAGCTTTAAAAAGACTTTTTAAAATCTGCTTTACTGACATATAGT
TTATGAACAATGAAAAGTATTAATTTTAAGCCTACTTGCTGTTCAATGAG
TTTTGACAAATGTATACAACCACACAACCACCATTTTAATCGAGATATAG
AACATTTCCATGACCACCAGAAAATTTCTTGATACTTCCTTCCAGACAAT
ACTATCCCCACCTCCTCACTCCACCCTCCTTCCTTGTGCAAGCAACCAAT
AAGCCTTATTTCACTATAGATTAGCTTAACCTGTTCTAGAATTTTATATA
ATCGGGGTCATATAATACATTATGTATCTAGTTTTTCTTGCTTAGCATGT
TTTTGAAATTCATTCGTGTGGCTATGTGTATAAGTAGTTCAGTCCTTTTC
ATTGCTTGGTAGTATTTCATTGGATAAATAGATCATAATTTGTTTTTCTG
TTCATTTATTTTTTGGCATTTAGGCAAAATATGTTTTTCTGTTCATTTGT
TTTTTGGCATTTGTTTATAATGTTTTTACTATTGTGAATAAAACTGCTTA
AAAATTAAGCAGTGCACATTTGTGCACAAGTCTTTCTGTGGACCTATGTT
TTCTTTTCTCTTGGGGAAATACTTAGGAGCAAAACTTGCTGGATCACAGG
GTATGTGTACATTTAACTTTGTAAGAAACTGCCTATTTCCAAAGTCATGC
CATTTAATGCTTCCCACTGGTATGGGAGAAGTCAGTTGCTCCGTATCATT
TTGAAAACTTGGCATCATTAGTGTTTTTAAATCATTGTCTTGAGGTTTAA
CTTACATGCAATAGTATCCATATATTTTTATTTTTATTTATTTTTTGAGA
CAGAGTCTCGCTCTGGTGCCCAGGCTGGAGTGCAGTAGTGCAATCTCAGT
TCACTGAAACCTCAGCCTCCTGGGTTCAAGCAATTCTCATGCCTCAGCCT
CCCGAGTAGCTGGGATTACAGATGCACACCACCACACCTGGCTCATTTTT
ATATTTTTAGTAGAGATGGGATTTTGCCATGTTGGCCAGGCTGGTCTTGA
GCTCCTGACCTCAAGTGATCCACCCACCTCAGCCTCCCAAAGTGCTGGGA
TTACAGGCATGAGCCCACCGCACCCAGCCAATATATTTGTAGAATAAGGT
AAATTTGTTCTGATAAATGTGTACATCCATGTGACTTCCATATCCAAATT
AAGATATAGAACATTTCCATCTCCCAAATAAGTTCTCTCCTGCTCCATTA
TGGTCTCCTCATTACCATACCTCTTCCCTGCAGGGAACCTCTGATCTGCT
GTCTATCACAGTAGATTAATTTTGCCTGTTCCGGAATTTAATGTAAGTGG
AATCTTAAGTATGTCCTCTCACCATAATGCCTTCGAGTGAGATTCATCCA
TGTTATTGTATGAATCAGTAGTTTATTCCTTCCCCCACATGCAAAATGAA
TTTTAACTTTAAAAATCGTTTTTAAATCTTTACATTATCAACATCTTTAA
TAGTCACGAAATCATTATTTCAAATGGCCTAAGCCCATCAGTTTGTGTTT
CAAATGATTTTACTACATCTTAAATATCTAATAAAATATGTAGTTTTAAA
AGTCACATATTTGTAGAATGAACCTAGTAAAGGCTTATCATGAAGACTGC
CTGTTGAATATTCCTGTTTTCCATTATATCCATAAGTAGCTTCTTAAGTA
GGATCAGATTATGACAATCAGATCATCTAAGTTTCTATGAAGGCAAAAAT
TTAGTGTCTGATTTTTCAGTTTTCACATAAAAATGCTGATAAGACTATCT
GAAAAAGTGAAAATACTATCTTAACTATCCTATGCTTGGTTAATTTTCCA
CAAGATATTTTTAATTGAATTTTTTGAGATATTTGTAGATTTGCATGCAG
TTGTAAGAAATAATATAGACAGTGGCCGGGCACAGTGGCTCACGCCTGTA
ATCCCAGCACTTTGAGAGGCTGAGGCTGGCAGTTCACCTGAGGTCAGGAG
TTCGAGGCCAGCCTGGCTAACATGGTGAAAGCCTGTCTCTACTAAAATAC
AAAAATTAGCTGGGTGTGGTGGTGGGCACCTGTATTCCCAGCTACTTGGG
AGGCTGAGGCAGGAGAATCGCTTGAACCCGGGACGGCGGAGGTTACAGTG
AGCCAAGATTGCACCACTGCACTCCACCCTGGGCGACAAGAGCAAGACTC
CATCTCAAAAAAAAAAAAAAGAAAAAGAAAAAAAAATACAGAGAGATCCC
TTGTACACTTTGCTTAATTACCCCCAGTTGTAACATTTTACAACACTATA
ACATTGCAGCCAGAATATCGACATTGATACAATCCATTTATTTTAGATTT
CTTCAGCTTTACTTGTACTCATTGGTGTGTGCATATATGTGTGTGTGTCT
ATAGCTCTACACAATTCTATCACCTGTGTAGCTGCATGTATCCACCACCA
CAGTGGAGATACTGAACACTTCCATCACCATAAAAATCTCTCCTGTTGCC
TTTTTTTTTTTTTTTTTTTTTTTTTTGAGACAGAGTTTCACTCTGTCACC
CAGGCTGGAGTGCAGTGGCGCAGTCTCAGCTCACTGCAACTTCTGCCCCC
GAGTTCAATTCTCCTGCCTCAGCCTCCTGAGTAGCTGGGATTACAGGCAC
CTGCCACTGCACCTGGCTAATGTTTGTGTGTTTGGTAGAGACAGGGTTTC
ACCACATTGGCCAGGCTTGTCTCAAACTCCTGACCTCAAGTAATCTGTCC
ACCTCGGCTTTCCAAAGTGCTGGGATTACAGGCGTGAGCCACTGCACCCA
GCCTCCTGTTGCCCTTTTATAAACACACTCACCTCCTTTCCTCCTGCCCC
CTCCTACCCCATCTCTAGCCCCTGGCAATCACACTAATCTATCCTTCATT
TCTAAAATGTAACTAATCTATCTTTCATTTCTAAAATCTATCCTTTGTTT
CTAAAATTCATTTCTAGAAGGTATATTCTAGAATGTATACACACACACAA
CATGGATGTATCTCACCCAGAAGGCTTAATGTGAGAGGACATATTTAAGA
TTCCACTTACATTAAATTCTAGAACAGGCAAAACTAATCTACAGTGATAG
ACATCAGATCAGTGGTTCCCTGGGGAGTAGAGGTATGGTAATGAGGATAC
CATATAGTATATATAATGATACACATAATATATGGTATATATAATCATAT
GGTATGTATTTTTTCACTCAATATCAATATAATTCATTGGACAGTCATTC
AAGTTGTTGCATGTATCAGTAGTTTGCTCTTTTGTATTACTGAGTAGTAT
TCCATGGTATGGATGTACCACAATTTGTTTAACCATTCACTTCTTTTCTT
TATATATATACTTTAAGTTCTGGGGTACATGTGCAGAATGTGGAGGTTTG
TTACATAGCTGTACATGTGCCATAGTGGTTTGCTGCACCCATCAACCCAT
CATCTACATTAGGTATTTCTCCTAATGCTATCCCTCCCCTAACCCCCGTA
ACCCCCTGACAGGCCCCAGAGTGTGATGATCCCCTCCCTGTGTCCATGTG
TTCTCATTGTTCAACTTCCACTTATGAGTGAGAACATGTGGTGTTGGGTT
TTCTGTTCTTATGTTAGTTTGCTGAGAATGATGTTAACCATTCTTTTCTT
GAAGGACATCTGGCTGATTTGAGTTTGAGGCTAATAATGAATAAAATATA
CTATGAATATTTGTATGTAGGTATTTGTGGGAAGAAAAGTTTTTATTTAT
CAGGGATAAATATCCAAGAGTGTAACTGCTGAGTCATACGATAATTGCAT
GTTTAATATTTTAAGGAATTGCTATTGTTTTACAGAGTGGCAATATCATT
TTACATTCTCACCAGTAATGCATGAGTGATCCAGTTTCTCTGTATTCTTG
TCAGCATTTGATATTGTCACCCTTTTAGTTTAGTGATTCTGATAGGTATG
TAGTAACATTTCATTGTGGTTTTAATTTGCATTGCCCTGATGGTGTTGAG
CATCTTTCATGTGCTTATTTACCATCTGTATATCCTCTTTGGTAAAATGT
CTGTTTACATCTTTTGCCCATTTTCTAATTACATTGTTTTTTAACTGTTG
TGTTTTGAGAGTTCTTTATGCTCTGTAAATATTAGTCCTTTGTTAGATGT
GTGATTGGCAAATTCTTTCTCCCAGTCTCTAGTTCATTTTTTCCATCCTC
TTCACCTGAACTTTCACAAAGCAAAAAGTTTCATTTTTATGAGATTTGAT
TTATTAATTTTTCCTCTTATGAATTCTAGTTTTGGTGCCAAGTTCATATT
ACTCAGTAGTTTTCCATTGTATAAATACCATATATCTACATGTTTCGGCC
TTTGACTTGTTGATGGACATTTGGGTTGCTTCCAGTTTGGGGGCCATTAT
GAACATTTGTGTTACTGTCCTTTTATATACATATGTTCTCATTTCTCTTG
GGTAAATACCTAGATGTGGAAGTGCTGGATCATATGGCGAGTATATATTT
GACTTTATAAGAAATATTAAACTATTTACCAAAGTTATAGAGTTGGATTA
CTACTTACCATTTTTGGCCTTCTTCATTACTTTCCATAGATCCAAGTTTC
TACCTGGAATCCTTTACCTTTAGCCTGAAGAACATCCCTTAGAATTTCTT
GTACCACAGTTCTCATGGTGACAAATTGTCTCAGCTTTTGTTAACCTGAA
AATGACTTTATTTAACTTATTTTTGACAGATGTTGTTTACTGGATATAAA
TATAAAGATCTGGGTTGGCAGTTTTTCTCTTTTGCACTTTAAAAATGTCA
TGTCATTGTCTTCTAGATTCCATTATTTACGCTGAGAAGTTAGCAGTCCA
TTTGTGCCATTGATCTCTGTATGCAGTGTGTCTTGTTATGTTTTGTTTTC
TAGCAGCTTTCAATATTTTCTCTTTATCTTTGGTTTTCAACCATTTGACA
TGATGTCCCTAAGTGTGCTTTTATTTATATTTATATTTACTTATATTTAT
CTTTTATTTATATTTATCTCTATATATTTATATTTTTAGTTCACTGGGCT
ATTTTATCTTGCTTGGGATTCACTGAGTTATTTTTCAATTGAGAAAATTT
CAGCCTTATATCTTGAAATATTTTTTCTAGCCCATATTTGTCTCATCTCC
TTCTAGAACTGTGATTAACCATATTGGACCATTTGATACATTCCACAGGT
CTTGTCCATTTTCCTTCATTTTTTTTCCCTCTGTGGTCCTCAGATTGGAT
AATTTCTATTGCTCTGTCTTAAGTTTATTTAGCTTTCTTCTGCCATCTTC
TGTTAACCCACTCTTATGACCATCCAGTGATTTTTTTAATTTCAGTTTTT
ATATTTTTCACTTACAGAAATACAATTTGATCCTTTTTTAATAACTTCCA
TTTCACTGCTGAGACTCCTGAACTATTCACTCATTAAAATAATATTCTCA
TTTAGTTGTTTGAATATATTTTCCTTTAATTCCTTATTTTTAATTTATAA
TTCCTTATTTATAATAGCTACTTTAAATTTTCTGTCTGCTAAATCCAACA
TCTGGGCCATCTCATGCTCAATTTCACTTGATTACTTTTATTTCTGGACT
TTAGGTCATGTTTTCGTTTTCCTTTGCTATTCTAGTTATTTTTTAGTATC
TGTTGGATATTATGAATGACATCCTGTAGAGACTCCAGATTCTGCTATCA
TCATCTAAAGAATGGTGATTTTTGTTCTAGTAGATAGTTCACTTATTAGC
TGACCACCTTGAACTTATGTAGGCTTGGTTTTATATACTTTGTCAGGAAA
AATCTATAAAAATCCCAGATATTTGTGAAGCCCTTCTAATTTATTGGGTC
TCAACCTCCAAATTCTGTTTCCTCTAAGGATCTCATTAGGGCTTGATTTT
AGGCATTTGCAAGGCAGGTCTAGAGTGTACTTTGTTCTAGGACATGTTCC
TACTCCTCCTCTAATAGCACAGGGGTGTGAAGGCTTGCTGCCCCATCCCA
ATGTTTTCAGGCTTTTGGTTTGTATAAGGTAGGAGATGAAGGAAATAGGT
GGTGTTTCAGCCATAGCCAATCACCTTCTGCATGTCTGCACCACCCAAGG
AAGGCTTCTCTGATCTCTTGTTCTGCCACGTCTTTCTAATAAGCACCCAG
GGAAGAACCATGGAGAATCTTGTGAGTGTGTGCAAATTCGTATCTGGAAC
TCTCAGTTATTCCCGATTGATATGTTAGACCAAACTCAGCTTTTAGGACT
TTATGAAAATATTAGCTGATTTCTTTCCACCCACTTAGATGGTGGCCTCC
TCTTCTTCCTGTGTTTCACTAAAGGTAAAATAGTTTTTGTGTTTCTGGGT
TGCCATGCAACCTCAACTCTCTGATGGATTTATGAAAAGTTATGCCTTTG
CAGGCTGCCTGAGCTTGTTGTTGTTAAGAATGGGAACAACATTCACGTGC
AGCTTTCTACCTCCCAGCAGAAGTGGAACACCTCAGTAATGTCCCCTTAT
AGAAAGACATTACATTGTACAAGTAGCACTAACCATAAAAAAAATTGATA
AATTAAACTACATTAAAAGTAAGTACTTATTTTCATTGAAAACGCCATTA
AAAGAGTGAAACAGCAAGCCATGGAGAAGAAAAGAAATTACCAACTTATA
CAGCAGACAAAAGATTTGTACCAGAATCTGTACACAAAACTTCTACATGT
AAACAATAAACAGATATACCCAATAGAGAAATGGGCAAAAGACTTAAACA
GATACTTCACAAAAGAGGATATCTCCAGCAAATATATTAAAATGTACTTA
ATATCATTAGTCATCAAGGCAATGAAAATTAAAGCCACAATATGAATCTA
CAATACCCCCACCAAAATGACTAAAGTTAAAAAGACTGATGATACTAAGT
GTTGGCAAGAATACGGAACAATAGGAGCTCTCATACATTGCTGGTGGAAG
TGTAAACTCGTGTATTCATTTTGAAGACATTTTGGCATTTTCTACTAAAG
TTGAATACTTGCATATCATCTAACCCAGAAATTACACTCCTAGGCACATA
CTCAACAGAAATGCACTGCACACGTGCACCAAGAGACGTTTAAGATGTGT
CCTAACAGCATTATTCATAGTAGCCCCAAACTGGAGACAATACCACACAT
ATACACAAACATATCCAATATACATACACCAATAAACATAAATGAACCAC
AGTAATCACAACATCATGGATGAACAGTACAAACATTTCATTGAGTGAAA
AACTAAAACACAAAAGAACACATAACCAATAATTTCAAAAACAGGCAAAA
CTAGACTATCATATTGTAAGTTATAAAAGTGGTTAAATTTGGGAAGGAGA
GAAATTTAGTAATGGGGAGGGGCATGAGGGAGGTTTCTATGACTGTGGCA
ACGTTCTACTTCTTGAATTCATGGTGGGAAATTGTTTTGTCATAATTCAT
TGAATTGTACATTTATACTTAATATACTTCTGTGTATTATATTCT

An exemplary DNA sequence is human Srprb provided below.

gi|224589815: 133497257-133545954 Homo sapiens
chromosome 3, GRCh37.p13Primary Assembly

(SEQ ID NO: 308)

CAGGAGCTGTGCAGGCAAGTAGAAAAGAGCACTGGGAGAACGGCCCAGTT
CACAGAAGAGGAGGCAGCAAGTCTGCACACTTTGTACTATGCTGAAATTG
TTCAATCACTCGACAGTTCAGAAATAAATATAGGAAGATTTTGAAAATCC
TACCTCTCTGCTCTGCTCCACAGCACTCCTGGAAGCCTGCACTTTCCGTA
GACCTTAAAATCTCAGAGGTAGGGCTGCCACCAAGGTGAAGATGGGAACG
CAGATGATCCATGAGTTTGCCCTGGTTTCACTGGCCCAAGTGGTTTGTGC
TAACCACGTCTGTCTTCACAGCTCTGTGTTGCCATGTGTGCTGAACAAAA
AATAAAAATTATTATTGATTTTATATTTCAAAAACTCCATTCTTTCCTAA
ATATTTTCAACAAAGGATTTCTTTATGCATTCTGCCTAAATACCTATGCA
ACTGAGCCCTTCCTTCTCAGCTCAAGATTCGTCTGGTCTTTCCCTACAGC
TTTGTGTGTGCCATGGCCACATCTCCTGGGTACAGTTCAAGGAGACATCT
TTTCTAAAAGGGTCTGCGTGATCATTAAAATATAATCAAATGTATACTGG
TGTGTGTGTGCACGTGCGCGTGCGTGTGTCATGCTAAGGAAGGGGCAAGA
AGGAGGATGCAGAGTGAGTTACAGAGATCAAGTACTCAAGTGAGTACAGA
AAGACTACAGAAGACTTACCTTTATTTGGTATTTTAAATGAATTCCATCA
GGCTGAGGTCCTGATTCTAACTAGCTGAAAAAAGTGAAACACTGACATCT
TTTTTGCTGAAAAGCATTCCCTGAGCTTGATGCCACAGCAGTTACTGCCC
TGTCTCCTGCCCTTCTCAGCCCAGCTTCCTGTAAGGTGGAGGCTGACTTT
TCTGTCTCACTTATTATTGGCTGAACATACAGAATTTGGTCTTCTGCTTC
ACCAACTTACCTGAATGTACCCTGGCAAAAGTCACCGTCTACCTGCCAAC
TGTTAAACCAATTCAGGTCTTCCATTCCTGAGCCCATTTCCTGCAGTTGA
TAAAGTTGACCACTCCCTCCTCCTTGGAACTCTTTTCCTCAGTTTCTCTG
ACATGGCAGGTCTCTGCTTTGATTCCTACCTCTCTCTGTTCATTTGTAGT
CTTCACTGTGGGCTCATCCTACTGGCCTGGAAACCTTAGGGAAATCTTCC
TGGGGTTTTCTCTTGATCTGGTCAGTGCTCCTCTCACTTTGCAGGCCCTC
CTTCAGTGATGCTCATGACTCCTAGGCATCTGTATCTATAGCTGTGACCC
CAGCCTCCAGCAGTATCCCTGCACTTATACATCCTACTACCTACCTGTGG
GTCTATACTCGAGTCTTGCAGCAGTCAGAAACCCAACAAATCAAAAATTG
AAGTGATCATCCTGTTCTTCTCTCTCTTGCTTCCCTAAACCAACTGTTGT
TATAGTATATTCCATATCAGCTGAAGACCTGGTCATCTACCCAATCACAT
AAGCCATAAATAGAGGGGTGAAATTTCAGTTCGCCATTTTCCCTCACACT
ACATCCAGCGTCACCAAGTTCTGTTGATTTTGCTTTCTAAATAACTTCAG
TTGGCTCCTTTCTTTTCCTGACTCCTAACACAGCCTCATCTTATACTCAA
ATACAGAAGTGCAGATAAGGGGTATCTGGACACAACATTCCACACCTCTC
TCAACTGTCTAGTATTCAAGAAAATAGGACTCTGGGTCCACTTTGCAGTC
CAGACCTGATATGGACCGTTTTACACACAGCCCTGCTTTCCTCTGAGCCT
ATCAACGATCTGCCTTATTTAATATTAACCTAAACTTACCCCAAATCCTA
TAACTCTGCCTTTTCCTTTGTTTGGTGAGATACCCCATGGCTCCCCTGGG
ACGCAGTCTGTCTCATTGCAATGGATCAATAAACCTAATTTTATTACACT
ACAGGTATATTCCTGGAAGTCTTAGACAACTGGGCTAAAACAAAAATATT
CAACATCCATTCTTAATTATTAAATTAGTTACTAAAATAGGATAGACAGA
TGTTATTTGGCCATAATTTTATATACATGTAAACAAAAGCTAATACATAC
CTAATGATGAAGAAACAGAAGCATTCCTACTAAAGTTAGGAACATGAAAA
AAATTGATTACTATCTATCTTATTTTACAGTGTTTTAGAACAGGTGGCCA
ATCCAATTATACAAGAAAAATAAGTTATAATATTTAAAGAAATGCCAAAA
TTACTTTTATTTGTAAGTGATTATACACCTAGGAAATCCATGAGAACCAA
ATAATAATAAAGTATTATGGCCAGGCGCAGTGGTTCATGCCTATAATCCC
AGCATTTTGGGAGGCTGAGGTGGGTGGATCACCTGAGGTCAGGAGTTCAA
GACCAGCCTGGCCAACATGGCGAAACCCCATCTCTTCTAAAAATACAAAA
ATTAGCCTGGTTTGGTGGCACACACCTGTAGTCCCAGCTACTCAGGAGGC
TGAAGCATGAGAAGTGCTTGATCCTGGGAGGCAGAGGTTGCAGTGAGCCA
AGGTCGCACCAGAGCACTCCAGCCTAGTGACAGAGCAAGACTCTGTCTGG
AAAATAATAAAAATAATAATAATAGGCAGGGCGTGGTGGCTCACACCTGT
AATCTCAGCACTTTGGGAGGCCAAGTCGGGCGGATCACCTGAGGTCAGGA
GTTCGAGACCAGCTTGACAACATGGAGAAACTCCATCTCTACTTAAAATA
AAAATTAGCCGGGCGTAGTGGTGCATGCCTGTAATCCCAGCTACTCGGGA
GGCTGAGGCGGGAGAATTGCTTGAACCTGGGAGGCAGAAGTTGTGGTGAG
CCGAGATCGCACCATTGCACTCCAGTCTAGACAACAAGAGCAAAACTCTG
TCTCAAAATAATAAAAATAATAATAATAATAAATAAAGTGGTAAAAGTGG
TAAGATAAAATTCAGTAAAGTGAAAGGAAATATTAAAATAAAAAATAACT
TTCTTTTATACAAATAATAGTGAGTTAGGACAATAATAATTAACAAGAAT
AAATAAAATGTATCATTTAGTAAATTAATAAAAGCTACATTTAAACCACA
CTTATCAAAGTAAAAAATAATGTAAAGAAGACATATTGCTTTGGATAGGA
AGACTCAGTATTGTAAAGATGTCAATTCTCCCTAAATTAATCAATATACA
ATTTTTTAACTTGACAAAATGATACTAATCCTCAACTAAAAAACAAAAGT
GAGACAGATAAGAAAAATTCTGATGAGAGTGGAAGAGCTCTTCCAGCTGT
TAAAATGTAATGATAAGCTCCTGAACCTTGGCCTTAGCAATGATTTTTTG
GCTATCACATCAAAAGCCCAGGCCACAAAAGCAAAAATAAATAAATGGGA
CTACATCAAGCTGAAAAGCTTCTGCATAGCAAAGACAACAAGCAACAAAA
TGAAATGGCAACCTATGGATTGGAAAAAATATTTGCAAACTATATATCTA
ATAAGATACAACAAATTGATAAAGAACTCATACAACTCAATAGCAGGAAA
ACAAATGATCCCAGTTAAAAATGGGCAAAAACCTGAAAAGACATTTCTCC
AAGAAGAAAAATGGCCAAGATGGATATGAAAAGGTGTTCAACATCACTAA
TCATAAGGGAAATGTGAATTGAAACCATTATGAGACACTAGATAATAATA
GGATGACTATTATCAAAAAGTCAAAAGATATTAATCATAAACGTGGATAA
GGGTGAAGAGAACAGAAAACCTTTGTGCATTGTTGGTGAGAATGTACAGC
CATTGTGGAAAACAGTATGGAGGTTTCTAAATAAACTGAAAATAGGGCTA
CATATGACCCAACAATCCCTCTTTAGGGGATATACACAAATAAAATGAGA
TAGCCACCTTGTAGAGATAAATGTGATCCCACGTTCATTGCAGCATTGCA
GCCAATATAGGGAAGCAACCTAAATGCCTGTTGAAGAATGAATGGATAAA
GAAACTGTATACACACACACACACACACAATGGAATATTATTCAGCCCTA
AAAAGAATATCTTGCCATTTGCCACAACATGGATGAAACTGGAGGATATT
AAGTGAAATAAGGCAGATACAGAAAGAAAAATATTACACGATTTCACTCA
CATGGGGAATCTAAAGAAAACACTCAAATACAGGTATACAGAGATAACAA
AACCTGAGGTGGAGGAGGGTGGAGAGATGTAGGTCAGAGGATACAAAGCA
GCAGATAGGAGGGCTAGGCGCAGTGGCTCACTCCTGTAATCCCAGGACTT
TGGGAGGCAGAGGTGGGCAGATTGCTTGAGTCCAGGAGTTTGAGACCAGC
CTAGGCAACATGACAAAACCCCATCTCTGCAAAAAAAAAAAAAAAAACAA
AAAAAACCCCAAAAAACCAAAACAACAACAAACAAACAAGCAAACAAAAC
ACACACACACAAAAAAAATTAGCGAGGAGTGCTGGTGCCCACCTGTAGTC
CCAGCTACTCAGGAGGATGGATTGAGCCTGGGAGGTCGAGGCTGCAGTGA
GCAGTGATTTGATCGTGCCACTGCACTCCAGCCTGGGGACAGGAGACCCC
GTCTCAAACAAAACAAAACCAAGTAGCAGATATGCATGATGAACAAGCCT
AGAGATCTAATGTACAACATAAGGACCACAGTATAGGTAATAAATTCTAC
TTTATTTGGGATTCCTCCTTCACTGTAGTAATCTTTTTTTAACTATGTAT
GTATCTCATAACATGTTGTATACCTTAAATATATTCAACGAAGCTTATTT
TAAAAAATGAAATGTATAATGATGACTGGAAAGGGTGGTAGTCACATGGA
AATAATGAAGCAGAATAAAAGTTCCAGATATTAAATGCAAATGAGAATTT
ATTACATGATAAAGCTGGCATTATACTCGGGGGGGACAGATAGGCTACTC
ACTGAAGTGTTTAGAAGCAGAGTAGCTGAAAATTTTGGCGCAGAAGAAAA
TCTCGTGGACCTCCAACTTACCGGTTAAAGTAAAAACTTTCGAAACGTAT
AAAGGTTTAAATATAAAATTAAAATCGTAAAAACAATTACTATGTTAACA
TATTTCTTATATAGTACTTAGATTTTCCAACATTCCACAAACCTAGTAGC
AATTAAGGAAAATATTAATTTCAGATCATATAAAATTCAGAAGTCCTGTA
AGACAAAAATAGACAAATAAAATGTGAAGATTTTTAAGAAAGAACAAAGG
GCTAATCTATATTTTGAAATCAGTATAAAAAGACGAACAGAATTTGAGAT
GTGAGCGCGGACAGCTCTGCTGGGCCCTCCAGGCCCTCCGCCCGGTAGAA
CCCGGAGCCCTGGCCGCGGCTGCCCCACCGGGCCCATTTCTTGAAGCAGC
GGCGAACCACTTTTCCTCCCCGACCTGCCGGCTGCAGGGTCTCCCCGCCC
AACCCTGCCAGGCTCAGACGAAGCTTCTGCTGAGCCGCCTGGACGCAGCG
CCCCTGGGTGGCGGCAGGTATAGCAGGGCGGGAAGCAGGGAGGCGGCCCC
AACGCCGCGGAGGCCACCAGCGGGTGCAGAGGGCCAGGAAAGCTGGTCTG
CGCGCGGCTCAGCTGCCTCCGTAGGCCATGGAGCCGAGCAGATCCGGGTC
TTCGGGTGGCCGCAATGTCCCAAGTGGACGACACCCACCTAGTCCCGAGG
ACAACCGACAAAGTCCTTAATGGGCCCAGAAGGTGAGGGGAGTCCGGCAA
TGAGGGGTGGTAGGGTTAGCGGCCATCGCCCAGCTCGTCTTCCTTCTACC
AGACGCTGGTGCTGGAAAAGAGAAGTGTAAGAATAACTTGCGCCATTAGG
CCCATCGGAAAGGCCCACCACCCTTTAGGAAGATTACTGGCTGTTTATAG
AAGGCCCGTGTATATCCTATGAAGAAGCTGCTCTCAACTTCCCCCCCAGC
CTTTTAAAAGAAAACATTTGCTACATCGAGCCGTTCTAGGTGTAAAGAGG
TTGTGACTTATGATAGAGTTAGAAAATCACACATCTTGTAAATTCCCATT
TGTTAAAAAAATAATAATAATAATAATAATAATAATAATAATAATAGGAC
ATAAATGGCTTCTGGAGATGACTTTTTGCAATGAAGTTGTTAGACCACCT
CTGGAAGTGACAGCAGGAGCCACACATCCACGTTTATTAAGTGAGTTAGA
GCAGGAGTCCCCAACCCCTGGATCTAGGTTGCACGCTCCTTATGAGACTC
TAATAATGCCTGATGATCTGAGGTGGAACAGTTTCATCCTGAAACCATGC
CCCTACCTTCCATGGAAAAATTGCCTTCCGTGAAACCATTCCCTGGTGCC
AAAGAGGTTGGGGACCACTGGGTTAGAGGACATGGGGCAGAAGACCTGAG
AAGGAAGAGAGGACAGTTCTGTGCCACACTGGTCATATTTAGAAGACATT
TTCATATTCCAACCATTGTTCTGTGTGTGCATTTTATTTCTCACGACTGT
ATATGTAGTTAACAATACTATTTTTTAGGGAGGTGGGGGGGTCAGCCCCC
CGCCTGGCCAGCCGCCCCGTCCGGGAGGGAGGTGGGGGGGTCAGCCCCCC
GCCCGGCCAGCCGCCCCGTCTGGGAGGTGAGGGGCGCCTCTGCCCGGCCG
CCCCTACTGGGAAGTGAGGAGCCCCTCTGCCAGGCCAGCCGCCCCGTCCG
GGAGGGAGGTGGGGGGGTCAGCCCCCCGCCCGGCCAGCCGCCCTGTCCGG
GAGGGAGGTGGGGGGGTCAGCCCTCCGCCCGGCCAGCCGCCCCGTCTGGG
AGGTGAGGGGCGCCTCTGCCCGGCCGCCCCTACTGGGAAGTGAGGAGCCC
CTCTGCCCGGCCAGCCGCCCCGTCCGGGAGGGAGGTGGGGGGGTCGGCCC
CCCGCCCGACCAGCCGCCCCATCCGGGAGGGAGGTGGGGGGGTCAGCCCC
CCGCCCGGCCAGCCGCCCTGTCCGGGAGGGAGGTGGGGGTGTCAGCCCCA
CGCCCGGCCAGCCGCCTCGTCCGGGAGGGAGGTGGGGGGGTCAGCCCCCC
GCCCGGCCAGCCGCCCCGTCCGGGAGGGAGGTGGGGGGGGTCAGCCCCCC
TGCCCGGCCAGTGGCCCCGTCCGGGAGGTGAGGGGCGCCTCTGCCCGGCT
GCCCCTACTGGGAAGTGAGGAGCCCCTCTGCCCGGCCAGCCGCCCGTCCG
GGAGGGAGAGTGGGGGGTCAGCCCCCCCCCGCCCGGCCAGCCGCCCTGTC
CGGGAGGTGAGGGGCGCCTCTGCCCAGCCACCACCCGGTCTGGGAGGTGT
GCCCAACAGCTCATTGAGAACGGGCCAGGATGACAATGGCGGCCTTGTGG
AATAGAAAGGCGGGAAAGGCGGGGAAAAGATTGAGAAATCGGATGGTTGC
CGTGTCTGTGTAGAAAGAAGTAGACATGGGAGACTTTTCATTTTGTTCTG
CACTAAGAAAAATTCCTCTGTCTTGGGATCCTGTTGATCTGTGACCTTAC
CCCCAACCCTGTGCTCTCTGAAACATGTGCTGTGTCCACTCAGGGTTAAA
TGGATTAAGGGCGGTGCAAGATGTGCTTTGTTAAACAGATGCTTGAAGGC
AGCATGCTCGTTAAGAGTCATCGCCAATCCCTAATCTCAAGTAATCAGGG
ACACAAACACTGCGGAAGGCCGCAGGGTCCTCTGCCTAGGAAAACCAGAG
ACCTTTGTTCACTTGTTTATCTGCTGACCTTCCCTCCACTATTGTCCCAT
GACCCTGCCAAATCCCCCTCTGTGAGAAACACCCAAGAATTATCAATAAA
AAAATAAATTAAAAAAAAAAAAAAAAAACAATACTATTTTTTGAACTATC
TACCCTTTCTAGATGTTCTGAAATGCCTATGTTAAAATTAGAGATAGTAA
AATAACACATTTTGTAAATCTTTTTGTTAAAATTCATATGTAATGTTGTT
TTGGTTGGGGGGGGTGGCCAAACCACCTGTTAATAATACACATTGTGTTT
GTGCACTGGTTCAGGGGAGGAGAGAGGAAAGGAAGTGTAAGAGCTCTATG
CCAATGTGCTTATAGTGAGGCAAGGTTAACCATTGTCTCATGCCTGGGCA
TTTTGTTTTACTTATCTGGGTATATTGTGGATCTAAAGGACAAATGAGTC
CTGACTTTACATCTAGTCTTTCTAGATGTTAAAGAGGTTGCTAGTGTATG
ACAAAAGTAGTTAGTAAACTAATATATTTTGTACATTTCGTTTTAAGATT
CCCAGGAAAGACAGTCTTCTGAAAATTTGAGCATTATTGCCCAGTGGGTT
GATGGAGATGGGAAGGTGTAGGCCAGAATGTTCATACTTGGAAGACTCAG
TCATAACTGTTTTTACATGTGTTCACTTTATTCAAGACTTCTGTGCATAT
AGTGGACGAATTAACTTCTTACCTGAAACATCTTATCTATGTAGATGTTC
AGAATTGCCTGATGTATGTTAAGTGTAGAGGTAGTAAAATATCACCTTGT
AAATATCTTTTTGCTAAAATTCTTGGGAAGTACTGTCTTTTGGAAGTGGA
ATTGTGAAGCCACCTCTGTAAGCAGTATAGTACTGTCTATACTTGTTCAA
TGGTTTAGAAATGGGAGGGAAGAAATTGCCAAAGGTAATATGCTAGTGTG
TTCATACTTGGACATTTCACTTGCCATTTTTCTGTCTGTTTTGTGCATTT
TGTTTTGCTGTGTATGTAGAGTACATAATGGACAAATGAGTCCTAATTTT
GCAACATTTGGTCTCTAGATGTTTAAGAAGTTGCCAGTGTATGACAAAGT
ATCTAGTAAAATTAGCACATTTTGCACACTTTGTATTGAAATTCATAGGA
AAGCTTGTCTTCTGTAAATGATTTTTGGGTATGAATTTGTTCAGCCATCT
CTAAGCGTTACACATGCCTGTACCTGTCCACTGGATTGAAGACAGAGAGA
AGGGAGTGAGGAAGGACTGGTTCAAGGTCAAAATTGTCATATTTAGAAGA
TACCTCCGATTATAGCCACTGATACATGTGCAATTTTATTTTACAGTGCT
ATGTACACAGTGGAAAGTTATATGAAATATCTGGTCTCTCTAGATAGTTA
GAAATGCTTGATGTATTTAAAAGTAGAAATAGTATAATAACACTTTTTGT
AAATAGCTTTTAAAAACTGATGGGAAATACTGTGTTTGCAAGTGGGATTG
TTAAATCACCTCTGTGAACAGTGTTACTTGTTCATTCAGTTGAGGAAGGT
GAGAGGGAAGAAATTACAAAAGATGTTTTGCTATTGTGTACTAGAAAATT
TCAGTTTATCCATTGCCTTATATGTTAAATGCATTGCAAGTAACTTTGCT
ATACTACATATTGTGTTATATACTGGAAAAATGAGTCCTAATTTTAAAAT
ATCCACTCTCTAGATACTAAAGAGGTTGCCAATGTATGACAAAAATAGTA
AAGTAACACATTTTGTACACTTTGTGTTAACGTTTATAAAAAGACTATCT
TCTGAAAACGACTTTTGGAAGTGAAATGATAACATCAACCCTAAGTGACA
CATGGGCCCATATCCACCAGGTTGGTGGTGCAGAGGAGCTGGAAGAAATG
AAGGATTCTAGATCAGAATATTCCTATTTAGAAGACAGTTTAAGATAAAC
CATGTGTGTAGTTTATTCAACCCTACTGATGCAAGAGAATAGGGTCTGGA
GGCAGGAAACATAAAGCAGATTCATGCAGACTTCCTAGAAATAAATGAAA
TGGAAACACATCAGCTATGACAGGACATATACTCTCCAACAGAGTAAATG
ACTTTGTAACTTTATTTCATCCTCTCCATTTACATAGGGCGTACATCAAG
TAAATGACTAGGGTGTATACTAAGTAAATGACTTTGTAACTTTATTTCAT
CCTCTTCATTTACATAAGGCATATACCAAGTAACCAATGGAAACCTTGAG
AGGGTATTTAAACCCGAGAAAATTCTGTAACTGGGCTCTTGAGCCCCTAA
GCACAGGCCTTCTCCCACCCTGTGGAGCGTACTTTCATTTCATAAATCTC
TGCTTTTGTTGTTTCATTCTTTCCTTGCTTTGTGTGTTTTGTCCAATTCT
TGGTTCAATATGCCCAGAACCTGGACACCCTCCACTGGTAACATATTTTG
GCAAGGCAGCCAGGAGGTAAGCCCAAAGTTTGGGATTTTTCATCTTTTCT
TTTCCACTCCATACAGGGGAATCTCTCCCTCTGTCTCTTTTTCCTTTCCA
ACTCGGGACCCTTGGTGGGCAGCGCCTAAACACGGAGGCAACCGCAGGTT
TCTGGGTGGGGCCATCCTGAAGGACTTTTTTCTATCTTTTCTGGTTGTGG
TCCCTGATTCCTATGTGTGGCGCAGCTCAGGGCAAACTTGCAGCTCAGGG
TGAACTTGCATGTGTTTCAGGCGACGGAAACCCTACTTGACTGGCTAAGG
ACAAAAGAAGCCTACTCAGCTTCCATTTCCTATCATTACAGTTCATGGCT
ATCATGCTAGTGGAACAGGAAGCATGGGATAATGTGGCCTTATCAAATTA
TAAGGATGCTAAACGCCAGTGATTACACCCAGGAACCAAAGGACAGCTCA
CAATTGCCTCTGGAGGGAAAATATGCAAAGCGGCACTGGTGCCCACGTAA
GGTCAGAGATGCCTGACACTCTTAAGACTGGACCCCAAAGGGGGATTCCC
CAGGGGATCCTCCAGACTTCAACCTCTCCAAAGGGGATGCCCTTGGCAGC
AGTTCTAAGGACTAGTACTAAGCCCTCCTTAGAATTTTCTCTTGCAGTTG
CAATACTGTTTGGCCCCAATATGGTTTGGATTCTGGCATTTGCTGTTGAA
TGGGAAAGCGGGATGCAGTTGCATGTATCCAGGCTTTGGTGCTGCTGTTC
TAAGCAGGGGCAGGCCTAGTTATTATGTGATGTTCTCCTTTGGTGCTGTT
TGACCCCAGTGTTCTTTCGAGTCTGGGGAGGTTTGGCCTTTAAAAATCAA
ACTGCCATGGAAATTGCTTTACCTGAAATAATAGTTCACAGCCTTCATTG
GATTGCTTATTGGGTACACAAAGTAAAATTGGCAAGCTTGTATTGCTATC
TCATGGTTAGGGTTCCAAGGTAAAAGCTATTGAATCTTCATTTGTGTGTG
TGTATACACATGTCTAGATGTGTTTATTTGCACATTTGTTATATGTTGTG
TTTTGGCATATAAGTAAAAGAGCACTCATAAATTAAGTAAATAAGTCGAA
GCAATTTTCAAGTTCACATGACTTAAGTATAACTTTCCTAAACAGGCTAG
TTTTAAAATTATTGGTGAAGTAAAAATAGAAATGCCTTCAGAATTGTCAG
CATATATTTTTGTCTGGGTTTTATATTTGTCTCTGCTAGATATTTTGAGG
TGTTAGGGTTTGGCATAGAAGGTTATAAAACTATAAACCCAGCCAAAACA
AAACGATCTCGTTTGCGTTTTTTTTTTTTTTTTTTTTTTTTTTTTTGACA
AATGAGCATAATTTAATGTTAGCTAAATCTTCTGGGTTACTGGCAAAAAT
ACGTATGTATTAACTTTGAGGCTCTTACTTAGGTTTAAGTGCACACCTGG
CTATTCAAAATGTGGTTAACAGGGAAATAACTGACTTTAAAAGATAGTGT
CTAACATTTCGGTTTACAGAGGTAATCTAGATAAACTGTTAAAAGTGAAA
CAATTGAGTACAGTGAATGGGACAAATGTTTTAGATAAGCTTTTTGTGTA
AAGTCTTAAAATCATTTTTGATGCTCATTTAAGATCTGGGCCATTTCCAA
TTAGAAAGGTTGTGATATGGGGAAATATGTTTCTAAAATTGTGGAATTGT
TCTTAACTATAAATTCCCATATCTGATAGTTCAGGATTTCTAGCTTTTTA
GCGTTTCACTAAAGTTTTAGGTTACTAAGGATAAGAATTCTAGTTAACAC
ATAATTCTGTATACAAAACATGCCAGAAAGTTTGTGTTATTAGTGAGAAA
AAGAATAATTTAGAAGTTATCTAAAAGTTAGTTCAAATTACAGATTTGAA
AAGGTTACTTATGAAACAATGTAGTAAGGAACCAGTAAGTAGAGGAGAAC
GATATGGAAAAAGTTTAGATAATAAAATATTCTTTAAAACCTGATAGAGA
ATTGGAGATATTTGGCTAATTAACATTTTCATAGTGAAAGCTGTTAGTCT
TGATAAAAGTAAAATAAGCATTGTAAAGAAATGCATCGGCAGTTTGGCAA
TTCCTTTTTACTATAGTTAAGCATGAAGCCGGATTTGGTGTGGAGCCAAA
TTTCACATACATGCTTGCATTGCTTCACACTGTTTACTGTTTTGTGTGGA
TAGTGCTGGCACTGGAGTACTTATTGGTTATGTGCCTATAGTGTATTTCT
TGATTGCACAGGATGTATGGTGATATTGGTGAACTTAAGGATACTGAATT
GTGTATCAGGAACAAAATATTCATTATGTGGGTTTTGGGGGGCCCTAGGT
AAACACTGTAGCCTCCAGGGTAGATTGAGAAGGAAAAATTTAGGGTTGGG
TTCCTATTTGTTTTTGCTTCTAATTTTCATTTGTTTGCCATTTATTCTCC
TCTGGCTTTGCTTGTGTATGCACATATAAAATCATATATATTTTTTTTAA
ATTTCTAGTGGAAGGCTTTTATTTGGTTCTGTGAATAGTTATTTTGTTCC
CTATGCATTTCTAGCAAGTCATCATTTGTTCCATTTATCTGGAATTCCTA
GGCTACCTTTGTCAGGCTGGCAGGAATTGATGGAGCACACCAGCTTTTTA
ACCTTGAACTAACTCAAATTCTGTTTATGATACTTTAAGTGTGTTGAGTA
TATTTTCATAAACAGAATTTGAGTCATATTTCTCTGTCTGCCCATTTTCT
CCAAAATTCGTAAACTATTTGTGAATATTCTTAATTCATGGCAATGTGTT
TGTTTGCATATAGTCAAGCAGGGTTCCTAGGGCTGCTTTGGGAGACAGAA
CCCAGAAACCTGGCATACTGGCAAAAGGGTAAGAATTTCTTACCAGCCAG
ACTCTGGTCTCTGTCTCTCTGTGCAAACTGCATAAATGAATAGTAAAAGT
CACTGTTTATCTCCTCTGTAAAGTTTTGATTAATATAAAAAAAGAATTCT
GAGGCCGATCTTAAGCTGTAGTGAATCTGGTGTGCTTTGTGTCTTTCTGT
ATTGTTTTGTCATAAAGAGGGGTACCTTAGAATAAAATGTGTGCTTAGGA
CCCCATAGACCTGCTGTTCAAGACACACCAGCAGACTGGTCAGTCATGTC
TTTGGGAGCTTGACCTTGTAACCATGTGGCCATGCTTTCTCTTTTCACAG
TGGCAGCCTGGGTTTAGGGTTCAATTCCTGGCTTAGGGAATGAGTCCTTT
ATCTTCTGTCTGTGTATTTATATGTGTTGTGTGTGTACTATAAAAGAGCA
TTAATTAATTAGCTTAATAATAATAAGAGCTTAAATCAAATATTTTATCA
GAAAAGTAAAAAATGTAATGCCTTCTATTTACTTCATGTAACTTAAGTAA
TCTTTGGGAAATAAAGACAGTTTTAAAGATTATTGGTAAAATAAAAATAT
CTTCAAAATGTAAATATTTAGTCTAAATTATGCAGGTCAGATATTAGATT
TGCAAAATGCTTTAAGGTCATAAACCGCTTCTTTGACTTTTAAAAATTGC
TCAATTTATTTTGGAGCATTAGATTCTAGATAAGGCCTGGGGACAAATAG
AATTAGCCATGTCCCCTAGCTATGCAAAGAAGGTTATAAAGAAAGGAGAT
TTTATATAAGAAAGGATCTTATATCGTAAATTCGTGTCCTAAAGTAAAAT
AAGCGGTTGTTAAAAAAGAGGGATGTTTAGGACAAGTTAGAAAGTCAAAG
CATGTCGTAGATGGTCTGTGTAAGTCATGAAATAATTTACAAAAGAGAAT
TTATGCAAGAAATATACAATTTAACGGTGATTAGACCTCCTAAATGCTTC
ATAAAATGCCACTATGACTCTTAACTCTACAACTTGCCTGCTTTACAGCT
CGGTAAGGCCTGAGACACGTGGAGTCAGATGCTGGAGTCAGACCTTAAGT
GTACTTCTCTCTGGGTCCTAGACTCCACAACTAATATATAATTAAAATCC
TTAACTTACCAAAGTTTTCACCAAAAGTAAAACTCGCTAAGAGTTAACAT
TGTAACATGTAAGTGAGACTACTGAAGCAACAGTTTTACATGCAAGTTAC
GTAAGAAAAGTGAATGCATTTTCAGTGAAAGATTATAACAAGGCATGGGA
ATGTAGATTTTTTTTTCTGTCTAAAGGGTTAAAGGATTGTTTTAAGTTAG
GATAAAGCTAAAAGTTTGAATGAGTTGTGGAAGATTTATAAAAATTAATA
TTGTAAAAAATTATGTGTGTGAACATATTGACTAAATTTAAAGGGGTATT
TTCTGGTTTTTCCATAAATTGAACATCAAAATAAAAGCACAAACAGGGTT
TTCTTAAAGCACCGATCTGCTCTTTAACAAACATTTGTAAAGGATTATAA
AAGGTTTATGAGAATCTCACCTTATGGTCAAACGGATTAAGATTGGAAAG
ATATGTCTACAAGGTTTTCTTAAAAATTGGGGTTGACATTAATAGCACAC
TAATGCAAGGGTGAAATTTGGCTTTCTCTCTTGAACAAGATTTTCATGCA
GTATTAAAAGATAATGAAAGATCTTTGTTTGCCTTTTGAATAAACTACCC
CCCCACCAAAAAAAAGGAAGGGAAAGACAAGAGACAGATTGTTTGTAAAG
TAAGTCTTCCATCTATCAACATGGAAAGGTTTTTGCCTTTTAAAACATTT
TTGAGTCATCATTTTGCTAAATGAATGACTATGGTAACCTGGAATTCCAT
TTCATAATGTCAAGTGTTTTAAATCTTTAACATATTTAATAGGCTTCCCA
AAATCAAATTTCAGCTTCAAAATTGTCTTTTCTAACCTCTAACTTTGAGA
TGCTGCAGAGGGCCCCTGAAGCATCCAAAAGAGAGGTAAACAGAATTATT
TGACATGTTTAATTACATGGGAAGCATTGTCAAAAAGAAAAAAATTTAAT
CTTCTTCAGGTTATATTTTAGTGAATAATATTAATATATGTTCCAAAATT
GTATGAGATTTCTAAAATTCCAATATGTCTGAGTATATGCTATCAATCAT
AATTATGATTATTATGTTATTGTAGACCACAGAAATAACCAAATTTCCTT
GTCAATTGTGTTTTTAACTATAACTATTTAAAGTCATTTCCACAGTTAAT
TGCTTAATGCTGATGCAGTTTCTGAAAACTTCACAAGCATGCAAAGTCCT
AGAATATGGTGTCTTTTAGGAGATTCATGAAAGATTGGAAAGGACCCTAA
AAAGCACTCTTGAATACAGGTTTCTAATAAGTTTAGAATTATATCATTTG
AACTGGGTAAGAATTCCTGGAACTTTAATGAAAAGACTGACTGAGTTCTA
AAACTGCTAACCCAAGTAGAACAAAAATTAATTAAATACCAAGAAAATAC
TTTGCCAGATTTTCATGCTAAATCAGCTGATACTGAAACTGTTTAGATAT
AAAATTTGAATAAACTCCATCATTTAAATCAAATTACCTATGATAACCCA
TCAGTTATCAGTGTTATGCACCTAATTTGGGGAAACAACTGGTATTCAAG
AGGATATGAGTCTAATGTTAATTAAGCATGAACTCATGGAGAACCAGGAT
GGCCACCTTGTCCTTCCTAAGTCGTTAAAGCTTTTGTTAGTAAAATTTCT
GCATTACATGACTCGTCACGGAAAAGATAAAATGATCCAAATTGAATATA
TTGATGTGGTGACTTATAAATTGCTAAAATAGTTTATAACCAATGTTTGG
TCCCATATTCCTGGGAAAACAATCAAAGCTTCAGGTACATCCAGTCACCT
GATAGGCCATTTAAACATTTTATAGAGGGATTTTATTCAATTGTTATTTT
CAGTGCATGTTTTCTGGTTGTATAAAAGCTTTCCCATGCAAGAGGGCTGA
TGTTATAACACTAAGTTATTATGCCACCAAGTATTTTCACAAGGTAAAGA
AAGCTTTTTATGGTTCACTGAGGACAATCAACTCCTTCACAACCTAGAAA
CTGAAGATTGTATCTTCTGAAAACATTGGATAAAGACTGTCCTTGCCATC
CACACTACAGCAAAACTTCTGGACTTTGAACTTTGGGTTCATGATCTCAC
AACTGAGAAGAGTCCCTCCACACTCTTGGAACTGTACACCCATTGGAACC
CTTAAGGTAAAGCTAACCAGGGAAGTTTCTCTCCCGAAGAAGATGGCATC
CTTGATGTGAACAGCTTTTCCCAAGATCATGAATCAAGACTTCTCTATTA
TCATGAGACTCTTATCTTTGAATATTTTTTCCTTGCTTTTGCCTCTATGA
AGAATAGAAGTGGAAAAAGGATCTGTTGTGTACAATTATGGGTTATACTT
TTATTTGTGAAGAATTTTGCAGCCAGCTTTAGGTCCTTTTTCCATGATTT
GGAATAAAAGAGGCAAATGTATCCCTCATAATAGGCTCCATAGCAGTTTA
CTGTAAAGGCTATGGTTACATAATAGACTAAATTATCTTAAGAAAGTTAT
GCTAAATAATAGAGTTGGCTAAACAGAAGAGTATCTGTGCAGCTGCTGGC
ACTCATGGCCTATGGAGAAAACATCAGGTATTATAGAGATTCAGTTGTAG
CAAATTAATGAAGAGACTGCTTAGTTAAGTGAGTAGACTCTTTAGCTCAT
TGTTTAATCTATTTGAGTTTAGGAGGTTTGGTTTATGGTGACCCTGGATA
AGGAGCATACTTACTCTCAACTCTTGGTATTATCCTCCTGATAGTCATAA
TAATAGTCTCCCTGGTGTGCTGTATTCTCTCAACGGTTTTAAATGTTTGC
ATGCTGCCACCTCTAGAATGTCAAATGGCCTCTCTTCAACTGGAATGACA
AGAGCTGAAAGAAATTGCAGCCATGAGGACACTGTAACCTACGAATGATG
TGCTGAGTCCAAAAGCCCAAAGTGTTGGTAACTGAGAGTGGTGCTAAGTA
AGGCCCTAAGTTTTGGTCACACTCTCACTTAAGTGAGAACCTGACCAAAA
AAGTGGAATTTTTTTTTTTTTTTTTTTTTTTGAGATGGAGTCTCACTCTG
TCACCCGGGCTGGAGTGCAGTGGCACGATCTCGGCTCACTGCAAGCTCCA
CTTCCCGGGTTCACGCCATTTTTCTGTCTCAGCCTCCCGAGTAGCTGGGA
CTACAGGCACCTGCCACCACGCCCGGCTAATTTTTTTGTATTTTTAGTGG
AGACGGGGTTTCACCATGTTAGCCAGGATGGTCTCGATCTCCTAACCTCA
TGATCTGCCCACCTCGGCCTCCCAAAGTGCTGGGATAACAGGCGTGAGCC
ACTGCACCTGGCCAAAAAGGGGAATTTTTTTTAAACAAAATTATGGGAGG
TCATTTTTTTGTACTGAGCCCATGCGCCAGGCCCCAACAGACCAAACTAA
ACCAAAACGGAGTCACGTATGCCAAGACCTTAAGGAAACACATAGATCCT
AGAACAGACCACAGGCCAGGTTTTGTTTTTCTCCTACAAATCTCTATAAC
AAACATTCCTGACAGCATAGATATCCACCCCCTGAGTTCCCATTAAATCT
TTTAACCAAATTAATTTCCTCTCGCCAAAAGACCATCAAGCTTCAGATGA
TCATGCAACAAAAGTTTCATCCAGTTCCACCACCCCTGGCCATCAAGGAG
CTACCCTGCCTTCACTAGAAAGAGCAGAGCAGGACTTCTGTGATCCTCAA
TAGGTAGGGACTAGGCCTCAAGCCAACATGAAGCAGTTACAAAAGAAAGA
CCATCGGCCCCTCCACCTCCCATAAAGATTTATGGGGATCCCATCTCTGG
AGGGAAATGAGGCAGAAGAATAGGGTCTGTAGGCAAGGAATATAAGGCAG
ATGCATGCTCACTTCCTAGAACTAAATCAAATGGAAACACTTCAGCTATG
ATGGGAAATGTCTTCTGTATTTACATAGGGCATACACTGAGTAACCAATG
GAAACCTCTAGAGGGTATTTAAACCCCAGAAAATTCTGTAACCGGGCTAT
TGAGCCCCTATGCTCAAGCCCTCTGCCACCCTGTGGAGTGTACTTTTGTT
TTCAATAAACCTCTGCTTTTGTTGCTTCATTCTTTCCTTGCTTTGTTTGT
GCATTTTGTCCAATTATTTGTTCAAGAAGCCAAGAACCTGGACACCCTCC
AGTAGTAACACTACTGGACATATAGTGGACAAAATTAAGTCCTTATTTGA
AACATCTAGTCTTCTAGATGTTTAGAAGTGCACACAGTATGTTAAAAGTA
GAGGTAGTAAATAACACATTTTGTAGATATCCTTTTGATTCATATGAAAC
ATTGTCTTTTGGAAATTGATCAAACCACTTCACATTATATTTATGCTGAT
TCAGGGAGGAAGGAGCAGAAGAAAGTGCAAAGGGCTTTATATCAGTGTGT
TTACAGTATGACACAATTGACTGTTGTCCCTTATATCTGCATTTCCTTTT
ACTTTGCTGTGTATACAAACAAACATTTACATAGCTTTGGAATTTTGAAT
TGGTAAATATTCATGATGTGTGAAAAAGCATGATACATACTGTATGATCC
CAATTGCATAACATTGGATGGTGTCCTAATTTATAACATCTAGTCTTTCT
AGATGTTAAAGAGATTGCCAGCATATAACAAAACTAGAGTTAGTAAACTA
ATACATTGAGTACACTTTGTGTTAAAAATTCATAGGAAAGATTGTTCTTA
AAAATGCTTCAAAAGTAGAATTGTTAAAATCCCCCCTAAGCATTACAGAT
GTTTATACCTGTCCACTGGATTGATAGAGATAGGAAGGGGAAGGGCTTTA
GGCCATGTTCCTATTTAGAAGACACATCCAAATTATAGCCTTGCTTTGTA
TGTGCACCATTTATTCAATGCTACTGTGTATAAAGTGGAAAACTCAAGTC
CTATTTAAAACACCTAGTCTTCTTAGATGTTTAAAAGTGCACAATATAGA
TTAAAAATAGAGGGCTAAAGTAACACCCTTTATGCATTTTTGTGGTTGGT
GTTATTTCCTAGGGATGGTTGCATTTGAGAATGGAATTTTTAAACTTGAT
GCTGACTGTTCATCAGGTGTCGGCAGGTGGGAAGAGGAGGAGGTATGCAG
GGAGAAGGGTTCTGGGCCCGTGAGTTAGATTAGTTCAGATGATCTAGCCA
TTGTTCTGTATGTGAATTTTAGTTAATATTGGTGTGTTTTAAAGAATAAA
CAAGTCTTAATGCTCAAGGTAGTTTAAAAGTGGAGGTAGTAAAATATCCC
TTTATAAATGTCCTTTTGTTAGTTTTTAGGAAGACCTGCCTTCTGGGAGT
AATCTTTGTTAATCCACCCCTTGGAGCTAGACATAGTCCTGTACTTAGTC
ACTGGCATGCTAGAAGAGGGAGAAGGGGAAGGGTGAAGGGAAGGGCTTTT
TGCTAGTATCTCGGTATCCAGAAGATAGTGTGAGATAACAGCCATAGGTC
TAACAGCATTTTAGTAAAGTGCCTGCATTTATTGTGGACAAAGTTTATTT
TTTGGAACACTTAACCTTTATGAATGTCCTGAGGAGACAATGTATGATTA
AAAGTAGAGCTAGCGAATTAGCCAATTTGTAAATATCTTTTTGTTATAAT
TGATAGAAAAGATGCATATTGGACATGGAATTGTTAAACCACCACTGAGC
AGTGTATATCAGGACTTGCTCATTAAGTTGGCAGCAGGGGGGCAAAAGGA
AGCATAAAGGGAGAGATATATGCAGATATACATTTACATTTTGATGATAG
CCACTGATGTATGTGCATCTCTTTCGGCTGTACTATAGGAATACATTAAG
TAATTCAGTGAAAATATGCCTTCTTGCTAATATTTTCATGGCATAGAACT
ACTAATGAATTCTCTTAGAAACATTATACTTAGTGTATTCTGTTGCTTTA
TGTTTCATTTTAAATTGAACATTAAGGGAATGCAGTATTTTAATTGGAAC
TCTGCCAATGCTTTTATCTCAAGGTATGATGCCATTTTTGTCTTTTATAA
AATTTTTGTTCCAAGAAAGGCAGGATTACATTTTTTCCTAATAGATTGAG
TTGGTGTAGTGTTTTCTTGACTATCAAAATACTCATATAGCTTTAGGATT
TTGAATTGGTAAATATTCATGATGTGTAAAAAAGCATGACACATAAAATT
GCATGATCTCAATCCCATAAATTTGGATGTTGTTGTCTATACACACACAG
GACCTAGAAGGACAAGTCAAACTGTAAACTGCCTGTGATTGTGGATGACT
TTGCTCTTTGCTTCATGTGTTTTCCAGTTTCCTAAAATGCACGTTAGCTT
TTTAAAAATAAAAGATATTTTAAAAACATTAAAAAAGAGATGAACATTGT
GATAAAAAATAGGCAAAAAATACTAGAGACAGTTACTAGATAACACACAT
AGAGCCAACACACATCTAAAATTTGAAGCAACTCACTCATAAAGAAATAG
AATGCATGAGAACATGCACTTTTTTAGATCAGCTAAAGTTAAAGAAAAAC
TACTAATACCCAGAGGTGGGAAAGGAATAGAGAAAACAGTTACGGTTACT
ACTTTTGATGTGACTCTAGATTGGAGAAATCTTTCTCTAGAGGGATGTTT
CGCCAAATCTATTATACTTCAAACACATATGTCCTTTATTCTTGATTGCA
CTTCTAGGAATATATCCCATGGAAGTACTCTCACAGGGTGTTTATAATAA
GGTGAAAAAATGAAATCCTAAATTTCCATCATTAGAGGACCACAGTGATT
TAGGATATATCTAAACACTGGGATACTCTGCAGCATTTACAGAGAGTGCA
GTAGAGCTATATGAACTGCTGGGGAAAGGTGGTTCAGTAAAACAAGTTTC
ATAATCAGATGTATAGTATATAATCATATATTTGAATAAAAACATGAACA
TATATGCTTGCGTGTGTGTGAAAGGACAGTTACATATGAGCAGTAGCCTT
GGAAGGACAGAGAAAAAAATAATTATTTTAATTTTGCATTTTTATGTACC
ATTCAACTTCTCAAAATGAAGATCTATTACCTCAATTACTTTTTTCAAGT
GGCTCTCGGGGAGAAGTTTAATAGTAAAATAATCTGAATTTTGCAGTTCC
TGGTATTATGCAATTGTGTATTTTAAGGTCTAAGGAAAGTATGCCCAGAT
AATTCTCCAAAGGTCCCAGCTAGATTCTGTTAATATCAAGAAGGATTTAG
AAGTAGAAGAGACCCCTTCTGTGTTTAACAGACAAAAAGTTATCCTTGAA
TGTGAAGGAAAAAGTGCCATTGGGTGGAAAAGGCTGGGAAGGATGAACAG
AAGTTGAATGGAATGTTCCCTTGTAGAACACCATGGTTCCTCTTCTCATC
TACGTCACTGTAACGCCAGCATGTGCACTTCCACATGCCAGCGAGCAGTA
ATGACAGCACGCTTTCAGTAAAGCCAGGACAGCTGTGGCTGACATCATCT
TCCTTCTTCCCCCAGGGACGAACCTAGAAGTGTGGCACTGTTCCTCTGTA
GTACCAGGCAGCCTATGACTGAGGCTCAGGGCTGTAATCTGGCCTGGCCT
TAAATCCCCCTCCTTGGTCTCAAAAGGTTAAGACATCATTTGTTCTCTGT
GCTGCTCCATCAGCAGATAGGGACCACCTAGGACCTCCGGGTGTTTCCTT
CCCAGATCAAGTCATTTAAATAAGTGCAAACTGGGTGCCTGCTAAGAGCC
AGCAGTTTTGGTATAGAGCCAGACCTTCCCTATGCCTACACCTGAAAAAT
GAATGATTACATTGTACAATGTATGCTGTGGGACTGTCCAAAGTATCAGA
GCTGAATAAGCTTAGATATGGTGCAATCAAGTGAGTGAGTCAGCTCTGAA
GTGGAGCTCTTTGTCAGGACCTCTTGTGTTTCCAGTGGACTCACCTTTCT
CCCCTTCCATCCTTTAAGGGACTGGAGGGAGCTGCCCAAGCTGCCTGGCC
AGATGTCTGTGGACTAAGTGTGACTGATGGATATAGGGGACTGATCCTAC
TTGTATCTGACGAAGAATATTGAATGATGTTAGAGAAAATCCTAAATGGT
GCCTCTATTCAGATGTAATTATTCTTACCAGCAGATTCTACTTTCTGAGA
AGCTAAAATTAGAAATTAAATTTGGTAGGCTAGGGTGACATGTCCTCAAA
CCCGCTAAGGCAGGGATGTGAAGCAGGAGGATGTCAGACAGGAGCCTGGG
GCACAGCAATGGACACAGTAGGCCTGGGATCATTCATTCAACAGTTGACC
AGTGCCTCCCATGAGCCAAGTACTGTTCAGGGATGGGGAATACAGAGGTG
CATACTATAATCTCTTTAAAAATAACGTGTTCGTTTGTATTTACTGCTCT
TATCCATACAGCCTTCTTATTAGTACATATGTACAGAGGAATGTCTTAAA
TATTTATCAGATACTATAGGTAGAAAAATAGAGGTGATTTGTGGCTTTCA
TTATTGCTTGAGTTTTTAATATAATAAGCATGTGTTAAATTCTGTTTTTA
AAAATCCTCTAAACGTTTTTTTAAACAAAGCCATAGCAGGGGAATCCCAT
GATTGGTATCCCTAGGTCTGAGGTATGTTCCATTGTACATTGGAGCTTTG
GGGAGCGGATGGAAGAAGGTGTCTGGTGAATGGGACATTTTAGTTTATGG
AAGAGAAGAGAAAAATTCCCTGACTACAACTGGTTTCATTGTAACTTTCC
ATCTGCTATTGGAATCAAGAACTAAAAACTTATCAGCAGGCAGCAGTCAG
TATCGCATGTCCTTCCTCTGCATTCGTGCCCACGGAGCTCCTGATGGAGA
TGGGTGAGTACAGGGAAGCAACTAATCTCACCCCTCCTGCTCATTCCTGA
GAATGGGAGTGCTAACTTCTCACTGAGTGTGGGAGCTGTGCACATTTCTT
CTGCCCAGGAAACATACTGAAGAAAAAAAGTCTCTTTTCTTGAACCTTTT
TTCTTCCTTTCCCAACTTCACCACCACGGCAGCCCTCTTCATTCACATAG
CTCTGACCAAATATCCTCAGCGAGTTAAGACAGTTACGGTTGGAACTCTC
CGTGTCTGGCTCTTCCTTCTTCCCTGCATCTTTGGTCCCCAGTCAGGAGG
TCTAATGGTAATATCCAGTGATTTTTCTAAAGAGGAATCAGTGGTTATAT
TATACAAGGGGTGTGTGCGTGTGTTTTGGGTGGTGAATACAGAGCAGCGG
TTAGGACCATAAGAAATAAGAGAAATGGAATGTGGCATGACCGCTGCCAG
TTCTGATATTTTTGAAATTGGTAATTTAAAATAAAGTAGTTGGTTTACTT
ACTTAAGCCTCACATCTTAACCGTCCTTCCTTGATCTGGAGTTCCTATCG
CCTACCTTTATCAATTAAAAATGCCTTGAAAATGAATCCTACTATGTTAA
AGCATACGGTTAAGTAGATAGAAGCCCAATTGCCTGGGATGAAGTCCCAA
GTCTGCTCATTACTATATATCCTTGGGGAGGTTACTCAACCCTGCTTCAG
TTTCCCCTCTGTAAAATTAGAAAATGAGTCAGTGTGAGAATTAAACAAAT
TAATACATTAGGCAGGGCGTGGTGGCTTGTGCCTGTAATCCCACCACTCT
GGGGGGCTGAAGCAGGAGGATCACTTGAGCCCAGGAGCTCAAGACCAGCC
CAGGCAGCAGAGAGAGACTGTGTCTCTACAAAAATAAAAATAAAAAAACT
ACCTGGGCATGGTGTTTGTGCCTGTAGTCCCAGCTATTTGGGAGGCTGAA
GCAGGAGGATCACTTGAGCCCAGGAGTTCCAGGTTACAGTGAGCCCTATT
GTCACTGCACTCCAGCCTGGGCAACAGAGTGAGACCCTGCCTGAAAAAAA
AAATTAAAAGTATTTAGAATGATGCTTGGCACCAGGAAGTGCTCATTAAC
ATTAGAAACTATCTTCATGCCCCACTGTCTTCCCCATACACCATAGTCTT
GCTTCCATCCCCACACTGCCCCATCCCTTGCTTATCCCCTTGAGCTCAGA
CTCATTCAAAGCCTTGTCAAAGGAGCCTCTCTGCTACAGAAGATGCTACT
GACCACTTATAGGACATGTCTGTTTTACTCCTGTGCCACTGACTCTTCCT
TCTTAGGCTTTTTTGTGGGTTCCTTTCCTCATTCTTCATGTTCTCCATGA
TTTTAATGACCACTTATTTATTCCTGACTCCCAAATGAAAATCTAGTATA
TACTTGTGGAACTTTATTTGTGTTTACTCATCACTTGCTTGACATAAGCT
TTTAGATTTCCCAGAAGCACCTAAAACTCAACATATAGAAAAGAGACAAT
TTTCATTCTGGCTACTCTTACGTTGGATCCACAGCCCCAAGCCCTCTCCC
GTTGAGGTCCCTAATTCATTTAATGGCTTTCTCATCTTCCTAGTCACCCA
CACCCACCACCTAGGATGCAGCCTTGATAACATTCCTTTTACTTAATTCA
CAATATCCAACTAATCACATCCTACGTATTTTAACTCTCAGACATCTCTC
AAATTTGTGCTCTCATTTTCATACCTATTTCTGTCTCTGTATTAGACTCT
GAGCATCCTTTGCCTAAATACTGCATCTTTTGCCTAAATGCTCTCAGCTG
GTCTCTCTGTCTGAAGTTTTTTTTTTCCTCTCAAGTCATAGTCCTCCATA
GATCTGTCAATTATCTATGTCACCCCAAATGAAACTTCTACACTTGATCT
TAGCCAAAAGGCCGAGAAGCGATCCCCAAATGAAACTTCTTATTGCATTC
TGGACAAACTCTTAAGTTCCTCAACATAGCACAGAAAATCCCTTCTGGTC
TGATTCCTGCCAACCTTTCTAGCTTCATCTCCTGTCACTCCTACTTAAAT
TACTGCTCTAATCAATTTTTATAGATTTTATAATTGATGATTTGGCATCT
GTTGATCACTAGGCCAAACTAACAGCCAACATTGTTTACATTTCCCCTGT
GGCCTGGCTTTTGAGGGTGTGCCTAAATCATATTGCAAATCTTCCTAATA
CAACCCTTAACATCTGTGTTTCGCTTCTTGCCCAATCAAAAACATCTCTA
TCTAGCTACTCGCTGACACATTAGTAATAAAGGAAGAGTCCCAGTTCCTT
TTTTTTTTTTCCTTTTTTTGGAGGCAGGGTCTTGCTCTGTTGCCCAGGCT
GGAGTGCAGTGGCATGTGATCACAGCTCACGGTAACCTGGAATTCCTGGG
CTCAAGCGCTCCTGCCTCAGTCTCCCAAGTAGCTGAGACTGCAGGCATAT
GCCACCATGCCTGGCTAATTATTTTTTAATTAAAGAAAATGTTTTAGGCC
GAGGTGGGTGGATCACGAGGTTAGGAGATCGAGACCATCCTGGCTAACAT
GGTGAAACCCCGTCTCTACTAAAAATACAAAAAATTAGCCGGGCATGGTG
GCAGGCGCCTGTAGTCCCAGCTACTCGGGAGGCTGAGGCAGGAGAATGGC
GTGAACCCGGGAGGCGGAGCTTGCAGTGAGCCGAGATCGCACCACTGCAC
TCCAGCCTGGGCAACAGAGCGAGACTCTGTCTCAAAAAAAAAAAAAAAAA
AAAAGAAAAAAGAAAATGTTTTTTGCAGAGACAGAGTCTCACTATGTTGC
CCAGGCTGGTCTTGAATTCTTGGCCTCAAGCAATCCTCCTGCCTTGGCCT
CCCAAAGCGCTGGGATTAATAGCTGTGAGTCACCGGGTCCTCCCCCAGTT
CCTCTCTTTTTCCTTGTCCCTGTGCATGGTGAGGTTTTGTGTGGCCCTGA
GTGGTATCAAGCTAAATAACAAGCAGAGAGGGACGCTCTAAAAGAAAATA
ATATTTATTCTGGAATAGGCATTGCAATGGGACTACGCATGCCATAGTCA
ACTATGTGCATATTCAGGGGGGTGTAAAGGAAGACAAAGATTTTTAAAGA
AAAAATGAGTCAGATTCCATAATTGTTTTAAGATAATTATCCTTGGCTAC
AAGGATCAAATGGCAAGGATGGCATCAGTACTAGGTTGGACAGGGAGTTG
CTGGGTAGCCGTCCTAACAGATGTATTTTTTTGTGTGTAAGGTTGCGATG
GCCTTTGTGCAAGTCTGTGGTTTTTGCAGTCTTTTGTGACAGTTTTTGTC
ATCAGGCATGGGTGCCTGAGAACCCTCCCTCCATGGCCTTTCACCAGCTC
CATTTTTCTGATTAAAAAAAAAAATACAAGTGACTCCATTTTGATGCTGA
CAACTTTTTCAATGGCAATGTGGAACTGTAATAAACTTTTTTCAATCTTT
CAGTGACACTGACATCTTCATGTCTCTACTCAGCCATACAATTAAATTAA
ATAATGTGTACATTTTAAAACAATGACAGCTGTTTAATGACAGAGACTGT
GAACTGGGACCCTCTAGGCTGGCCACTTTTGGTGTTTTATTTGGGCCACA
AGTTTTTGAATGCTTTCAATTACCTACAGAAAATTTCAAAAGTGGAAAAT
TTCCTATAAAAATCTGCCTTTATTGCTTCTCTTGAAAACTGGAAGAACCG
GCCACCCAGGTCCCATAACACCTACCTGGCTGAATAGGGTCGGCCCTACT
TTCAGCAGAAATACCTCCTGCAGTTTTCAACAGCCCCAGCTCTCCTGGGC
GTTGATTTTCCTAGTTGCTTCCTTTAATGCAGACATTTGAGTTTGCAGCC
CTAGATTGACAGTGTAGGGGCACTGAATTACTTAGGGTTCCCACATACAT
CGTTTCTTACACTCATTTTCTTCTGCACAGCACCTTTTTTTCCTTTAACC
ACTCGCAGATGCTAACTCATTCTTCAGGACTCAATTCCAATGTCCAGAAG
CTTTTCTCTGAACTCATGATGGACTCCGCGTCCCATTATGTATTCCCACA
TCGCTCCGTACATCCATCTGTCACATCCCTTGTCACATTAAATTCAGATT
AAATGTTTGTAGGATGCTTGAGTTCTTCACAAGTCTCACGTCACTTAGCG
ACGGTCGGGAACTCCTCAGTGGTAGTTTCGGGAGCCATTCATTCATTTTA
TTCATTTCATCATTCTTTCAACAGTCATAACGTAGGTGACGACGACTGAC
TCATCTCTTAAAAGATGTGTGTGTCCCTCGACAAATAACCTGTCTGCCCC
TATCGTTCTGTAGGAGGACGGAGTCCCAGAGAACTACAACTCCCATCAAG
CACTTGGGGGATCGCCGCGGCTGAGGGAGAGACTATGGCTTAGGAACCAC
CATTCGCGTGAGGCTCTGCTCCCCGCGCCTGCGCAGAGTGCAGGGCCACG
TCGCTTTTGCTGTACCGGGGACCACGCGTCTCATCCATGGCTTCCGCGGA
CTCGCGCCGGGTGGCAGATGGCGGCGGTGCCGGGGGCACCTTCCAGCCCT
ACCTAGACACCTTGCGGCAGGAGCTGCAGCAGACGGACCCAACGCTGTTG
TCAGTAGTGGTGGCGGTTCTTGCGGTGCTGCTGACGCTAGGTAAAAGGCG
GCCGGTGGTCATGGCGGGTTTGGGGCGGGCAGAGGTCCGGGCTTTGGCTG
CACCGCTGCAGGCCGGGGACGCGGGGCTGAGAGGGCGCCTTGAGGGCATC
AGGAGGGTGAGACCCACCCAGTCTACACCCCACCCTCTCTCCTGAAGCAG
CGGCAGCAGCTGGGCGTTAAGGGGGATGCCGGGCAACCCCCGGAGTTAAC
GTCCTGGTCCTTGGGGCGCAGATTGCTCTTCGTCATCTGCCCTGTTACGC
TTGAGCGCGCTGTTCCTCGCCTTCCGCTGTTCTCACTGCAAAGACTCAGG
GTCTTGACACCAACTTTCAGCCTCCCGTGTTTGCGTGCGGGCGCTGGGAT
ACCAAGGTAGTGAGATAGCACCTGAGTCCCAGTCAGAGTAAGGACTGCAA
AGAAAAGTTGACAGTTGACAGTGCCTGGCTCTCAGTGGATCCCTAACAAC
AGCAATAGTAACCATTATGATAAAAGTCAGTTCCAGGTGAATGAGGTGTT
TCGTCACAGACTTCTGTGAATTTCCCCACCCCAGCCATGCACATATACTG
ATTCCCAAGGCGTAATTTTTGTTGTTTTTCTCTGTCTATTCCACAGTCTT
CTGGAAGTTAATCCGGAGCAGAAGGAGCAGTCAGAGAGCTGTTCTTCTTG
TTGGCCTTTGTGATTCCGGGAAAACGTTGCTCTTTGTCAGGGTAAATGAT
TTCATTGACACCCCTGTTAAGCTTAATAGAAAATTTTATAGATCCCAGTA
TTCCTGTCATCTCACGGTTTATTTTGTAAAAGAAGCTGATGCTGTTAAAT
TTGGAAGTTAATTCTTGACTTACTTGGGAGAACAAAAAGACTCGTAGAAA
CCCATAATTGAGAAATATTGGTCATTTCTCCTTTTTTTTTTCTGTTTGCC
CTTGCCAGATGCTGATATTTAGTCTTTATCACATTCAAGGATTTTATTTA
ATTAGTTTCATTCCAGAATAGCAGAAGGCATAGAAGCTAGTGTCCTACAG
TTTATAATTCCTTCGTTAACAAATTCTTAGGACAGTTTATCGAAGACACT
TTTTCACAGGGTGATCCTTCTTTGACATAGTGGGCTAGTTAGGCTTCTTT
TTACTAATATTTATTTTTAATACATTTAGTATTTTTAACAGTGTATTTTA
ATGTGGTGAATAGGAGAGGTTTTCTGCTTTTTAACTCTTCTTTTTGAGAG
TATAAATTATTTCCCTTCCATTAAAGAAATACCTCCATTTTTTTTTTTTT
TTTTTTTTTGAGGGAGTGTCTCACTCCAATGCCCAGGCTGGAGTGCAGTG
GCGAGATCTCTGCTCATTGCAGCCTTGACCTCTGGGACTCAGATGATTCT
CCCACCTCAGCCTCCGAGTAGCTGGGACTACAGGCGTGCAACACTACGCC
CGGCTGATTTTTTATATTTTTTGTAGAGATGAGATTTTGCCATGTTGCCC
AGGCTTCAAATCATTTTTGTTGGTTAAAAATCTTTACAGAAGCTTGGCAA
ATTTGGCCACTAACTGTCTTTTGAGAATAATGTTGTTTTTATTCCACTGA
GGAGTAAAATATGCAATTAATTAGATTTTTTTCTACAGGTAAAAAAAATC
AATATCGTCTTTTTCACCTGTCGGAAGCAGCATTCATTTACTTAACCTTA
CACCTGGGAGACTTAGAATAATATAACTCAATTTAGGTGTGCTATTAAAA
TGTTGAATATCACCCATCTTGTTTTTTTTACAGTTGTTAACAGGCCTTTA
TAGAGACACTCAGACGTCCATTACTGACAGCTGTGCTGTATACAGAGTCA
ACAATAACAGGGTAAGATGTTTGTGGAGTTTTGGAGTCTGACAGTCTTAC
TTTACTGTGGTGTGTCAGTTAAGGACATTTTTAGGATGAGTTCATCTGAG
GATGTAAGTACCATTTGTAGCTTATTTTTATGGAGAAACATATTCTTTGT
GACAAATTCTCTTGACAATCTGTACAGTAGATTTAATCATGCCCTCCTCT
GCATTGTTGTACCTTAATGTACAGTAATGTTATATCACATTTTATTCTTA
TTTTTTGTTTCTGTGTCTTCTTTCCAACTTTACAATGAGTTCTTAAGAGT
GGGCATTAAACCTTTTAAGTTTTGAATAGGTAATACATGCACATGGGACA
AAAATTCAAAAGGTTAGAAAAGATATTTGGTGAAAAGTAATTTTTCCTTT
CCTTTCCCCACTACCTAGTTTTCTCCTCAGAAGCAGTGACTTACTTTTTT
TTGTATGTAGTCTTCCGGATGTTCTGTGCATATACAAACATGTCTGTATA
TCCTTTATTGTTGTTTTTTTTTAAATGCTGTTCTGCCCATTATTTTTCTC
ATACGGCTGATACAACTTGGGCTTTTCTTATTGTCCTACATTTGTATACA
TAGTACCATCTGGTTGGTTTATGAGCTGCATAGTACTCCACTCTGTTGCT
ATACCATAAAATTAAATTCTTACTTAATCTGGACCGTGTTAATAGACATT
TAGTTTTGTTATTATAACCTTTGCTTTTACAAACAGTGCTGCAATGGATA
TCTTACAGAGTATATATGAGTGTATTTCAAATATAAGGAATCATGGGGCC
TGGCTTGGTGGCTCACGCCTGTAATCCCAGCACTTTGGGAGACCAAGGTA
GGCCGATCACTTGAGGTCAGGAGTTCAAGACCAGCCTGGCCAACATGATG
AAATCCTGTCTCTACTAAAAATACAAAAATTAGCCGGGTGTGGTGGTGGG
CGCCTGTAACCCAGCTACTCGGGAGGCTGAGGCATGAGAATCATTTTAAC
CCGGGAGGTGGAGGTGGCAGTGAGCCAAGATTGCGCCACTGCACTCCAGC
CTGGGCAATAGAGCGAGACTAAGTCTCAAAAAAAAAAAAAAAATATAAGG
AATCATGGGAAAGACATTTTACATACCTCTGTAACTTCAGAATGTAGACA
GTGACTACCAAACCGATTGTAATGCATAAGTTTGTTTGTTTGTTTTTGAG
ACAGGAGTCTCACTCTGTTGCCCAGGCTGGAGTGCAGTGGCATGATCTTG
GCTCACTGCAACCTCTGCCTCCCGAGTTGAAGCGATTCTCCTGCCTCAGC
CTCCTGAGTAGCTGGGATTACAGGCGCCCACCACCATGCCTGGCTAATTT
TTGTATTTTTAGTAGAGATGGGGTTTCACCATGTTGGCCAGGCTGGTCTT
GAACTCCTGACCTCAAGTGATCCGCCCACCTCAGCTTCCCAAATTGCTGG
GATTACAGGCGTGAGCCACTGCACCCAGCCATGTAATAAGTTTCGGTGAG
TGAGTGGAGTGAGGGCTCGTTGGAGGATGTGGATGGATAGATGGATGAAT
TAGAATTGTGGTTTGAGACCTTTTCTTCTTCTCTTTGTCTTCTTTCCCCT
TTCTCATGCCTCCAGAATTAAAGATTGTAAAGAAAAATAAAGGGACATGT
TTTTAAATATCTTAGCGATATCTTTGTTTTTTTTTTCAATGATGACACTC
TTTGTGGCCCAGACCTTTTTAAACTCCCAAGGATTCAGATTTTGAAATTC
CAGGTTTGCAAGATCATCTAGTACCTGCACTAGATTTTTAACTTAGGATA
CTCCAGGATATGCTTTAAATCTGTACCTTCTAATATGTAGCCACTGGCCA
CATGTGACTGTTGAGCACCTAAACTAGGACTAGTCCAAATTGAGGCATGC
TGTAAGTGTGAAGTACACACCAGATCTTGAAGACTTAGTATGGAAAAAAA
GAACGTAAAGTATCATTAATAATTTCTTTTTTTATTACATTTTAAAAGAT
AATATTTTGGATATATTAGATTAAATAACTTAAAATTAATTTTACCAATT
TCTTTTTACTTTTTTATTATGGTTACTGGAATATTTAAAATTACATATGT
GGCTTCTGTTATTCTATATTTCTACTATATTTCTATTCTATCAATATTTC
TACTGAATATATAGAGCATTTTTCCCTTCCCTCTTCACTCTTTCTCCTAC
CCCCTTACGTTTTCGATCTAATCTCTTTAGATAGTAATATACCTAGTAAT
GTATCTTCCAAATTGCTTAGGGTGTGTGGTATAGTGCTGCTTTTGAAATG
GAGCAGTTAAGTCCCAACCTGGGATCTCCAGACTCTTAACAGTACTTAAA
AAAAAAAGAGAGGGACTAATAAAAAACATATTTGTCTATAAAGAAATGCA
CAGGCTATGTGAAAGGTAAGGTCATAATAAACCCAGCTGGAATTGTGTTA
AATCTGTGCAGGATACTGATTGCCTGCTTCTGAACTGTGGGTATGGTTGG
CTGTGGATAATGTAGAACATGTAAGAAATGATGGTCTGCTGCATGTACCA
GGCCACATCTAAGAGTCTTATTGTTGAGCACAGCCACGTTTACACTGAAT
ATAAACTTTATTCACCATTCCAGGCCTTGACCTTATATTCAGGAATGGCA
GCCTGGCATGGTGAATAGAACTTATTAGGGTGCATCGTAGATTTGCTCAG
TAGGGGCTCAGGAATTGACACAAATGCTGACGGAGTTGGAACAAGTAAAT
TATATGTCTGGAAGATAAATATTTAAATAATTAGATTAAGGGTGGAAATG
ATAAAAGTGTACTTTGTTATAAAGATTGGAGATCCCAGTAAGAGTAATAT
GCTCTATGAAAAATTGTAAGTTCTTATATTACATTATTAGTTTATGAGTA
CTTGTTACTGTTTTCACATTTTTGCTTATACTCATACAGTACTCCTACTT
TGCTATACTCACACTGGTTTATCTCCCAAGCCTCAGGTAGATCATGCTTC
CCTGACTCCCCCAGACAGGTTTTTCCTGTACCCTCAAAACATTTATCAGT
GTTCTCATTTTATATTTATGTGTAATTTTACATTATGTTCACCTTTCTCA
TAAATTTTGCCAGATCAGGAACCGTGTCTTTGGCCACTGTTACATCCCCA
GAACGTTGACTAGTGTTTTATGAGTAGAAGATGCTTTGTGAACATTTGTT
TGGTTAATTAGGTACCAGTTTGGTAGCCAATATATGATACTAAAGGTTTA
TATTGTGAACGTGGAACTGTATTGAAACGTTAATGTTATTGCTGCCATCC
CCAGGGCAATAGTCTGACCTTGATTGACCTTCCCGGCCATGAGAGTTTGA
GGCTTCAGTTCTTAGAGCGGTTTAAGTCTTCAGCCAGGTAAGAAGGAAAG
AAGTGAAGTGGGCTTGTCTAGGTCTGTATCTGTATATCAAAGCCACTCAT
GTGATTTGCTCTGGTGGTGTTTGGGAGGCAGCATGGTATCCTGTGGTAGA
CCTTGAGGTGACCTTGGGGTCAGCCAGTTCTGGGTTGCAAGTCCACCTTC
ACCTGGATGATGAGGATTTTATGGGATTAAAATGCTTAAAAAGTGTTTTT
TAGTGTTTAGGAAATGTTAACCCTTCTTGTCATTGTTACTAAATGTTTGC
ATTTATTCTGAAGGTGAATTTAATAATGTATATGACTATTAAAATCTGAA
AAAAGAATTTTAAAGCTGGGATGAATCCTAGCAGTTTTTAAATGCCAACA
TCATTTATATATATAGTGTTTTTTTGTTTTTTTTTTTTGAGATGGAGTCT
CACTCTGTTGTCCTGGCTGGAGTGCAGTGGTGTGATCTCGGCTCACTGCA
ACCTCCACCTCCTGGGTTCAAGTGATTCTCCTGCCTCAGCCTCCTGAGTA
GCTGGGATTACAGGCACGCACCACCACACCCTGCTAATTTTTCATAATTT
TAGTAGAGATGGGGTTTCACCATGTTGGCCAGGCTTGTCTCGAACTCCTG
ACCTCATGATCCGCTGGCCTCAGCCTCCCAAAGTGCTGGAATTACAGGCG
TGAGCCACTGCACCTGGCCCATATATATTCTCAAATTACGTAAAATTGAT
GTTTCAAGAAGTACAGAACAATATTTCCATATTTCTTTTAGTGTGTGCTC
TGGTGTTACTGACTCTTAAGTTCTTCCCTAAAAAGTCTTATGCTGCAGTT
TCAGATGCTTCTTTTTTGGTCTCTAACATTAATAAGAACTGAAGAGGAAA
ATCAGTATGCCACATTTCACTGATTTGAAGACACATTTTAATATCTCTGA
ATTTAGAATATATCTTATAGTTGATGGTGTCATAGTTTAATTGGCAGTAT
TTTCTCTTAGCGGTCCCTAAAATAATGTCTTTAATTGGCAGCATTTTAGT
TTTGATGGAATGAAGTAGTAGATTCTTTGCCTTTTATTGAAGATGTCTAG
ACTGAAATTTCATTATTGCATCTTTGCCCACAAGATGGTGGTGATGAACA
ATTTGTCACAGATAAGAAAATTCCTTGGACAATTGAGACAAATCTAGAGC
TGGTAAATTTTGCCATAATTGGGTTTTTATTTCCAATTAAATGTTTTTTC
TTTTTCTATTGCCAGTTCTAAATATTTTATGATTTGCAGTATAACTACTC
TGTGATTCATGTATTTTGGGTAAATAAAACATGCAAAATATGAAGATAAA
ACTTAGTTCAAATAAATTATAATCATGATAACATGCAAGTTGAAAAGACA
TTTACTATATTGGTATGTGACTTTTTTTTAATCCTACTTTCTGGCTTCGC
CAAGTTTCCCTAGTGGCTATTTTCCCTCTCCTGATTTGAACATATGCTGT
TGCTGTTCTTATCATGGTTCCCCTCCCCCATCTTTTAAACATTGCCTATT
TGATTGTCTGAATCCAATAATATCAGTTTCCTTTTCCTGAACCTTAAACA
CTTTAACTTTCATCTTCCTCACATCTTCCTTTTATTTATGCTCCTTTTTA
ACCCCCATCCATACTTTTTTTTCCTCTTACATTCAGTGATGAGAATTACC
AGGATGCTTATGGTCTTTTGCTCCCCATTGCTTCTTGAATTTTACTTCTT
CCCTTTAGATTCAGTTTTCCCTCACTGAGTATATCTTTTAGAAGTGTTTC
CAGAGAGGGTGATGAGTAGTCAAATGTTTTCAGTCTTTGTTCATTTGTAA
CTCTATTTGGCCAGCATTTTGGAATGTCTCTTTTAGCTGAGTGCAGAGTC
CTGGGTGGCAGCCGCATCACTCCTGACCTCATCCATCTGTTGCAGATGAG
AATGTTTTTGCCAGTCAGTCTAATTGTCATTTCACTGTAGTAAGTCTGCC
TGATTTGACCTTCTATCCAGGGATAGATTTTCTTCTGTTCAAGGATGGAT
TTAGCTTTATCATTCTTGGAATTCAGTTGTGCTATTCCAAACCAAAGATT
CTCCTCAGTTTTGGAAAATTCTCACCATCGTTTCTCCAGGTAGTGCTGTT
TCCCCATGATCATTTCTCACACTCTGGAATTCTTATGAATGTTATTGAAC
CTTATTATTTTTGCCTTTCTTGTTTCCTCTCTATCGTATCTTCCAGATCT
TTCTACTTCGCATATTGTAAGATTCCCTCAGATTTGTTTTCTAGTTTATT
ACATTATTTTCAGCTGAGCATGATCTCTGCTTAACTTTTCCATTGATTTA
TTAATATCATTTACTATATTTGTCTAAAATACTTACCTTTTCAAACCAGC
CTGTTTCTTTTTATACTATTCTATTTTTTATTGTAGTTTGTAGTATTTTC
TTAAATAATTTTAAACATTTACTTTATAGATAGCTTTATTATCTTAGTTT
TGGGGTGCTGATTATTCTCTTTGGGGTACCAGTTATTCTCCCTCATAATT
ATTTTATTCTGTGCCTTGTAATTTTTGCTCCAGAACCTTTCTTCAAGAGT
GACACATCATTGATATTGTGGTGGTTCTTTAATCCCGTAGGATATGAAAG
CCTTGCTCCATAAGCGGTTCATAGTTCCTTCTGTTGGGGCCATAGGGCTT
CAATAAGTCTGGCCCAATTTTTATGTAAGCCTTTCCCCTTGGGGGTCTTA
CACCTGGAGAGAGTATAAATTCATACCCTATATCTGTGTGAGGCAGTGGA
GGCCCGCTGTTTCTAAGGGAACTTTTTTTGCAAGCCCCGACCTCCCTGAG
ACTATTTTAAGTTGTAGGCTTTGTGCAGGGCATTCAGTTCTAGCTCCCCA
GCTGCATGTTGGCCATTGCATGTCTCTTATCTCCACCTGGGCATTCAAAC
CCTTGTCCATAAGGCTTGTATTTTTTTTTTTTTTTTTGAGACGGAGTCTC
GCTCTGTTGCCCAGGCTGGAGTGCAGTGGCGCGATCTTGGCTCGCTGCAA
GCTCTGCCTCCCAGGTTTACGCCATTCTCCTGCCTCAGTCCCCTGAGTAG
CTGGGACAACAGGCACCCGCCATCACACCAAGCTAATTTTTTGTATTTTT
AGGAGAGACAGGGTTTCGCCGTGTTAGCCAGGATGGTCTCGATCTCCTGA
CCTCGTGATCCGCCCGCCTCGGCTTCCCAAAGTGCTGGGATTACAGGCGT
GAGCCACCGCGCCTGGCCGGTTTTTTTGTTTTTTTGTTTTCTTTGAGATG
GAGTTTCGCTTTGTCACCCAGGCTGGAGTGCAGTGGCATGATCTCAGTTC
ACTGCAACCTTCGCCTTCTGGGTTCAAGTAACTCTTCTGCCTCAGACTCT
CAAGTAGCTGGGATTACAGGCTCCCGCCACCATATCTGGCTAATTGTTAT
ATTTTTAGTAGAGATTGGGTTTCATCATGTTGCCCAGACTTGTCTCGAAC
TCCTGACCTCAAGTGATCTGCCTGTCTCGGCCCCCCAAAGTGCTGGGATT
ACAGGCGTGAGCCACCGCACCCGGCCCCCTAAGACCTGTTGTCTGCAGTT
TCCCTGGGCTGCTGCACTTTAGCTTTGATATCTCTTTGTTTCTGTCACAT
GAGGAGTCCCACTATGGCTGCCTTAGTTCAGGCCCTACCTGGTCACTGTG
CTTCTAGTTCAAGCGCCCTCTAAGATTGCTTTGTCTTCCATTTAGGCACC
AATATTCTTCATAAGACAGAAAAGCATGAACTCCACAGGTCATTGTGTCA
CGCCTCACTGTCTGACTTGGTTCTCCTTCATTTCCCCATCTCCCTGTGTA
CCAGCTTACCTTGACTTCCCCAGGGAATGTGTTTCACGTATTTGTGGCTT
GAACATACTGTAGGCTGAGTATCCCTTATCCAAAATGCTTGGAACCAGAA
GTGTTTTGGATTTTAGACTTTGGATTTTTGGATTGGGGATACTCAACCTG
TACTTTCCAGACAGCCTGTGCCCCAATTTTTGGTCTAGCTAAGATTCAAC
TCAACTGTTACATTCTCCTTAAAGTTGCCTTTGACCTGGTCTCATTTAAA
TGATCTTTCTCTTTATTCCTGTTACACATGTGCATAGTTTTGTCTTGGCA
TTTATCATATGGTTCTCTATATGATGATTTGTCTTGGAGGACAGGCATCA
TTTCTTACTGTCTTTTTTCAGCTTCAGTCATTGATACACAGTAGCACTCA
ATAGTTCCTTGTTAAATGAGGATTTATGTTATATGATAACATATGCACAG
ACCTGCATGTGTGTCTGCTGAGTGTTAATCACCAAGATTGACTTTGAAGA
AGTTCAGGATAGTTTTTCAAGATAATGATGTCCTGTTACACGTTCACATG
CCAGTTTTTCTTGTTTTCTCCCTCCAGGGCTATTGTGTTTGTTGTGGATA
GTGCAGCATTCCAGCGAGAGGTGAAAGATGTGGCTGAGTTTCTGTATCAA
GTCCTCATTGACAGTATGGGTCTGAAGAATACACCATCATTCTTAATAGC
CTGCAATAAGCAAGGTGCCATCATGTTTTCTTGCAATAATAATTGAGTTT
TTTGGGGATTGCTGCTACTAAGAATTATTTCCATTATTTACAGTTGTTAT
TATTGAGGATGAGATACAATTGCTGTAAAGAACTATAAATTGAAGGATTT
TTAATCTTAAAATTTATTTCTACTCCTACCTTTTCACCAGTCTTCTAACC
AGAAGACCAAGGAATGATAACCAAGTGACTGCTAACTGATTTTTCTTTGA
TACAGTGTTTTTTCTGATTATAAATTATTTTACTTTACCAAGTTAAGTTC
ATCAGATTGACAATATGTGATATCAGATGCACAGTTTTGCAGGGTGGAGT
TTCTGCTTTCAAACAAGTTTACTGTACAGCACATTTTATCAAGGGAAGTT
TGCTGAAGTTGTTTTCTTTTAACCTGATAACCATTGAAAACTTTTCTAGT
AATCCCCAATAAAACTGGGCTTCAGTTCCGTTACCTGTACAATAAGACAG
ACTAGTGGATCTCTTCAGGTCTCTTCCAGATCTGAAATTCTGATTCTATG
AAGAGTTCAAGGCATTTCTCAGAAGTGACCAGATAATATTGATAATGCCT
TATTGGGAATGCAATTAGCGTCATTACCTGACCTCTTTATTTCCCTGATT
GAATATAAGGAAGGGAGTTATTGTTTATTTGCCATGGGCTTTGTGCCAAA
CCAGAGCTTGTTTCTTACCTTTAGTACACTTAATGCTTTAAACATACCGT
CTTAGGATCAATTGGAAACATGCTAAGTTTACTTTAAAATGACAGATAGG
GCACTGTCTTTTGGCATGTGAAGGCACTTCTCATAATCACAGCACTGGTA
GGTTTTCTTTCTGTTAAACTGAGTAAACTTATATGTTTTGAATTGAAAGG
TCTATAAGGTGTGGAGAACTTTAGGGAAAGCTAAAGTCAGTTTTAGGGTC
ACCAGCATAATATGTTTATAAGCATTATCTTGGGAAAGCCCCTAGTGCAA
GTATAAAGCTTACTTTGATAGAGAAGGAACTAGTGATAAAAGGTCTTTTT
TTAAAAAAAGAGAAAAACTTACTGGTTTAAATAGGAATTTTCCTTCTGTG
TAAGAAGTCTTGGGGAAAAAACTCATTCTCGTTTAAACTACAACAGTGTC
TTTATTTCTTTACAGATATTGCAATGGCAAAATCAGCAAAGTTAATTCAA
CAGCAGCTGGAGAAAGAACTGTAAGTGTGAAAGAGGCCTGTTGGTTAATT
ATATATCTTAACACTTAGACTGTAAGCAGCATATTCATGTTTCTTTTGTT
TGGTTTTATGTGATTATCACAATTATAGACTGAAGATTAGTGTGATAAAT
ATTTGTTAAATAATTTACAAAATAATGGAATCACAAAAACTTACTAGGGT
GCTTATAGATATTTTAAAATTTCATGGAAAGAAGATTTTTTTGAAACCAT
AAACTTAATTTATTTTACCATTGTTACTAAATCATTTATAGTTGAAAGGT
TTTAGAAGCTAAAAAAGTTTACATGCCAACTTGAGAACGGTGTTATTTCA
GTGAGTGGAAGTGTTTGTTCACCACTCAATAATGCCTGTGAAGTGGTATT
TAAGGATGCATGTAAATAATAAAAAATAAAAACTGAGAATCAGGAAAAAA
GAAATTAAAACATTTAAAAGCCACTTTGGAAATTATTGTTGACAGAATAA
GAACAGAACACTGAGGAAGACTACAAGTCTTTGATACTACATTATAGCTT
CTTAGATTGTTAAAATACTTGGCAATATTATATATCCTGCTTTGAGTGTT
TCAAGGTTTGTAAATTGAGCTATAAGCTTTTGAATTTTAAGGCATCTAGC
ATGTCTTCTGCGTTATGCCAATTTGTAAGAGCTAGCCAAAAGCTGGTGTA
GAAGAAATGATCAACATTTATAGTAAAGCAAGTTATGTTTTCTTATTGCT
TTTGTGTAAGGCCTTGAAAAGGATCTGATGAGGAAAAACTGTATAAATTC
AACTTTTAGAAACTTTTCTCCCCACTATGACTTACTGACTCCCTTATTCA
TTTAAGAAGCACCTGTAGTGCATCTAGTGGATGCCAAGACTGCAGTTGGG
AGCAGCTGTAGGAGGAAATGTGTTCCTGTACTCAGGAGCTTAGAGTCTGG
TAGAATCTAGTCAGCTTCGGGCATTGGCAAACTCCTGCCTGGGCCTGCAA
GCCAAGAGTAAATCGACATTTTTAAAGGGCAGGTAAAAAACAGAAGAAAA
ATATGCGACAAAATATTTAATTTCTGGCTCTTTGCAGAACAATTTTGCCA
GCCCTGCCTATGCCATTTTCAGTGGTTCCTTTCCCGTCTGGAGCTGCGAG
TTTATCACCCTGTCCTCCTGTGCTCTTGATGCTCACTCTCTTGGATTGTA
TCTGTTGCTGCCATTAGGTTAATAAGGACATTGGAACAATTGGATGGTTG
GGTTTTTCTAAAAATTCTCTTGAAATTGTTGGGAATTGTTTTGGGGAAGC
CAAAACTGTCATAATGTTATTTGTGAATCAGTCTTGGGTTATTTTTAACA
GTACAGTCCAAGTCTCCCGTGGTTAAAACACATGTTTTCCATAGGCTGTT
TGCTTTTCACTCTATAGACTTAGAGAAAAACAATACAGAAGCCAATCCTT
TTTTAAAAGCATTGAAAGGCAATGCTTTCTTAAAAGGCAGTGAAACAAAA
TGTTGACAAATTGTAAAGGTTAACATCAGTTAAAATTCGGAATCTGACTA
ATTTTAATCTCAGATTTATGAGAATTAGGCCCTGTTAGCTTAGAGGCATT
TCATAGTCTCTCAATAACACGGTATATTATATTTACATGGCCTTGTGGCA
AAAGCTGAATACATGTTTTACTTTTTTCCTTAAAAAATTTTTATTGTAAA
ATATCTTACTTATAATAGTATATATGTCACATGCATTAAGCTTAAAAAAT
ATTAAAATGAATACCTATGTACTCACCACCATGCCTAGAAGGCCCCTGTT
TCTCCCCAAAAGCTTTTCCCTCAAGAGGTCAGCAATATCCAGTTTTGTAT
TGTTTTTTAAAAATACTTTTACAGTGTGTGTGTGTGTGTGTGTGTGTGTG
TGTGTGTGTGTTCCTTCCTAAACAAGGTATTGCTTAGTTTTGCCTGTGGA
TTCTTCACTAATTTTGAAGCAACTTTACAGAACAAGAAATGCTTTATTTT
GTAAGAAAGAAGATTGGCAGCATGTATTCCTCTGCTTCTGGATAGCAGAA
AGGGGAAGGGGAGGCCGACAAATGAGTGTTGCTTATGCACCCTTTATTAG
GGCCAGCAGGTGGCAGTAAGGGGACTTACATTGGGAAGGTTTTGGTGGTT
AAAGAGGTTGGATAATAGTAATTCTGCCCTAGTTAGATATGAGAATCTGG
AGTTGGTGGATTTCTAAACTCCATATCCAAATAGACTCAAGATAAAAATA
CATACCCTATCTACCCACACTTAACTTTCAGAAACTTATGTTTTTATAAC
ACTTAAAGCATGTATTTTAAAAATATATTTTTAGACGGCTTCATTCAGTA
GATCCTCTTAGAAGGGGGCCTTGGGCTGAAAGAAAGTTGGTGTGACCTGG
TCTACTGCTGACCTGCCTTCTAGCCCTGTGAGGTCTGACCCCCCCAGCCC
GCCCCACCTACCTCACCTCTCTGTCCAAGAGAGGGACTTATAAATGCAAA
GGATTATTCAACAATCTTAATAGCATAATTGGCAATTCTATAGTTAAGTT
TTAAATGAGTGCTGAAATGTTTAGAAACTAATATGATTTAATTGCTGTAT
AAAAATTTTGCCTCCTGACTTCTTTCCTTTTGCCCCACAGCAACACCTTA
CGAGTTACCCGTTCTGCTGCCCCCAGCACACTGGACAGTTCCAGCACTGC
CCCTGCTCAGCTGGGGAAGAAAGGCAAAGAGTTTGAATTCTCACAGTTGC
CCCTCAAAGTGGAGTTCCTGGAGTGCAGTGCCAAGGGTGGAAGAGGGGAC
GTGGGCTCTGCTGACATCCAGGACTTGGAGAAATGGCTGGCTAAAATTGC
CTGAGAGGCAGCTCTAAAGCACAAGACCTGGATGTGTGACACACAGTTTT
GGAAAAAGGTCTGTGGTAGTCTGGAGTTGATGAGGAAGGGGTACAAGATG
TGGTTAGAAACATTTCTTTGTTCTGGAAACAAAGTACTGTTGAAACCAGC
TTGGAATTTTTTTTTTTTTTTTTTTTAAGTTCAGTTCTCCCTTATGGCTG
CCTTTCAAACAAGTACCTTTTATCTGATGCCTGTATCTTCCCTTTGTTAA
GGTGTAACTTGATGTAGGGTCAAGGTTTTTGTGACAACAGGCAGACTCCA
CACAGAGAGGATATGATGAGAATATGGCCATCACCTGAAAAGTTTTCTTA
TCTTCTGTGCTTTTGGTCCCTGGAAACAAATCCGCCTATGTATGAAGCTA
GTTGATTTCCAGTTGCACTATTTCCAGTTGCCTCTGAAGTTCACAGGCAA
TACATTGTCTAGTCCTTTGCGAATTTCTCTGATTTGTGGGCACAGTTATG
AAGTTTCCCCACATGTGAAGACAGGTACAAAATAGCAGAGCCAAGCAGAC
AGTGGGTCTATTCTTCATTAGCTCAGTGACTTGTCCACACTCGTCTTAGC
ACTTACGTTTCAAAAGCTTGTCACAAACCCTTGGAGTCATTCCCAGATAA
TAGAACTGGAAATGATAAATCCCCTAATGCCAAGGGTCTAGTGTGTTCTT
AGTGGTTATACTGGGAAGTGTGTGGAGATTTAGGTGCTGCTCTGCTGCTC
TGGATGGCTGAAGGCTCCTGGGCCATCTTCATGTGCTGCTTGAAGAGCTC
CTATTTTGTACTCCTGGCTAGAATGCTGTGGAACAAATACAAAGTGAAAA
AAGTTCTCTGTAGATTTCTGAAGTGCATATTCATTGATGCCAAGAAAAAA
AAAAAGTTGCCTTTTTGAAGTGATGTTTTTTGCTGTCTTCTTAAACACAA
GGCTTTTTTGAATGATTAGTATATTTCATGGTAAAGAAAACAGCCTGTCT
GGCTCAAAGCAATTAAATAGAATGTAATGGTGAGTACAAATGAGTGCACA
TGTCAGGACTCAGGTCTAACTCCTTGTCTCCTGAGCCTAAAGATTGCAAC
ATACACAAGAACACACTCCTATTCCTACCCCACACACTCAGGGACAAGCC
CAACTAAAGCTTACAAGGAGACCAGGGTGGCTCTGTCCAGGGGAGAAGCC
AGTTATGGAACAGTGCATTGAGAGCCATGGTAGGAGAGGCCCACAGTTCT
CTGGAGCATGCAGCAGGGGCACCCCACCTGGCCTTGAGGATCAGGGGGAG
TCAAAGGATAAAGCATGGGGCTGATGACGTCTGAGGGAGTGTGATCCTCC
ATGTATGGCCTCTGCCTGCTGTCTCACATGTCCCTTCTGGTGGTCACTTG
GGCTCTAGGAGTATACGTCACCTCAGACCATCTGGCAGAAATACTCCAGG
CTCCTACCCCAAAGCACATGTCAGCCTTGCTGCTGGAGCACGAAGACAAT
GTAAATGAAACATGAAATGGAGGAGTTGTGAGACCCTGACCCTGAGTCCT
TACTTGAAAGCTGCTGCTGGTGTTCTGAGTGTCTTTTGGACTCTTATTTC
TTGCCCTTTTCCTTATTAGGCAAGCAGTAACTTAGGAAGTAGGTAAGAGC
AATAAATGTGACATGTTATGTCATCATAGTAGGAGCTCATGGGAATAAAA
GTCAGTGGCTTGATGCTTCTGTTAGAGGCATGTGTCCCTCTCAGCCTCTT
TTATTTTTTTCTGGTTAATATCTTACTTTATTGGAGGAAGCAGCACACCA
GAAAAAGTGACTGAAGAAGGGCTGCAAGTTTGAGTTTGATTCAAGGCCTA
AAGAAGGTCTTCCAAAAAAGGATAATTCTATCTGGAACACTGGTCTCAAA
TGTTAGTGGGCATCAGAATTACCTGCGATGCTTGTTAAACTGATTTGAGG
ACCCTACTCTCAGACAATTTGATTCAATAGCAAGTCTGGTATTGCCTCAG
GAATCTGCATGCTTAATAAGTCTTTTGTCAGTCGCATGGCACCCCTAGAG
ATGATCTGGCCTGGAGTGTCACGTGTGCCTTTACTTTGTTTTTAAAGTCT
CTTGGGGCACACACCCTGCTGAGTTTTTCTGACAGGGTCAAGTCAGCAGG
CATGGGAACTCTGACCTTGCTTCTTACTCCTGTACTAGTAGCAGGCATTC
CGTTCTTGTGGCTTTGGGCCAGGATGAGGTCATGGTGACTAGTCTTTGGG
AACTGGGAAGGGTTTTGATGGGAGGTCAAATGTGCTCTCTCCACTCAGGT
GGTGCCCTTTTGTGGCCCTGAGGGCATTTGACTAAAGTAGGCCCATTTCA
TGAGATGGGCAGGTGTGGCCAGAGTAGAAGGATGGATGTCAGGTCTTGGA
GACGGGCCAACTAGGCACGACTGTATGACATAGATGAGACTTTTGAGCTT
GAAACACCTTGGGAACATGGCAGTGTATGGACACCGAGACCTCCTAGGAC
AGCAGCTGCCTAATGCAAATGACCCTGTTCCCTGCTAAGGCTGCAGTGAG
ATGCCCTCATGGAGGGGCAGCTGTGATGAGTGAGCCCCAGGCACTCTGTT
GGTGAATATGTCCACTTCTGGAATGGAATCTCCCTAGTGGGTATGGACAC
GGGCAGACCCAGAGGGGTCTAAGCACTGCACTGTTTCTTCCTTTGGCTCC
TCTCCAGTCCGGGCCTTGGTAGCCTCTCCACTACCAGGTGCCTGACAGTC
TACTGGGGGTTCCTAATCAACAGCAGCTGCTGTTGGTGGGGAGTCCCTGG
GGTCTGTCACTTCTTCCCACTGCCTCTGTCTCAGTGCCTCCTGGGCTGTC
AGTGTTCCCCTGTGGTCCTGCCTTGTATGAAGCGCTGGCCATGCTCTGTT
GACTTAGATCATTTCAGCCAGTCTATGTTTTTTCTTGTTTTTGAAGTTTT
GGGTTCAGCACAATTGTCAAAGCTTCAGTTGGCTCATGCCTGTCCTCACC
TGAAGGCCATCAGTGGCAATTAAGATGCCTATCTCTTTATCTCATCCCTT
CACACATTTAAGGAAAGGTGATCACCCACCTACCTTCATTTAAATGCGCA
GGAAAACAATCCCACTTCCCTTCAACTGTAAACCTCGAGAAACTTGACTG
CTCACAAGAGCCTTGTACGCATTTATCCATTGGGTAAAGGCCACGCCTTG
CAGCAGACCATGGAAATACTAAACAAAACGCAATTCCTGCTTTAGAGGCT
AGTAGCCCCATTCTGAGGTTAAGAAACGTGCCCTTTCAGTGTGGTTTCCC
TGCTCTCTCAGTAGTGTGAGCTCCATCACTGCTGCACTTGACTCCTAAGC
GCTGTGTGGGCTTGTGGTCAGCTGCCAGCCATGCAGCCCCAACGGGACTG
CCACTTTTCCACTGGCCTCTGAAGTGCCAGTGTTCCCATGATGCTGATGT
GTGAGGCACATGGCCATGTTCTGTTGACTTGAATATATTGTTTCAAGGGT
TTTTTTGTTTTGTCGAGACAGAGTCTCACTCTGTCGCCCAGGCTGGAGTG
GCAGTGGTGCCGTCTCGGCTCACTGCAACCTCTGCCTCCCGGGTTCAAGT
GATTCTTCTGCCTTAGCCTCCCCAAGTAGCTGGGATTACAGGCCTGTGGG
ACCACTCCTGGCTAATTTTTAGTAGAGATGAGGTTTCACCATGTTGGCCA
GTCTGTTCTCAAACTCCTGGCCTCAATGTCTCTTCTAATCAAAGATGGCT
TCCTTGAGGTGGCAGCAGATTGGAGGGTGTTCTGGAACCTTTATAAGCTT
TAAAAGGAAAGGTAATTAGTGAAGTTGGACTTGAAGAAGAGTGAGATTCC
CGGGCAAGGGCAATTTACCTAATCTTGGCCTTTGTTCCTCTTTGGATTGG
TCCCAGGACTGTGCTGGCTTTCTGAGCATCTTTTAGTCTGGAGATAACTG
AGACCTGGAGGGGCCACTCAGGGATGTTCCTAACTGGTGTGGTGTGGGGC
TGGGGATGGAATATGGCAGCTAGATGTTGTCAAAGGTCATTATAGTTCAC
CTTAAGTTCTTGCCTTTAAATGGCTTTCTGAAAATCACCAGAAGCATGAG
AGATCCAGTCCCTGTACCCTCTCCCCCCTTCCTAATCCCCTAGCCATGGC
AAGCAGAGTGGGGATGAAGGAATATATCGTACCCCTGACTCAGTTCTGCC
TGGAGGCTCCCTTCCCATCAGCCTTTCAGCAGAATGGAGATTAGGGGAAT
GCGTGTGGATTTACTCTCATGAGTGTGTCCCATCTTTACCCCTTCGGAGT
TGGTGGACAGCCCAACTCCACCAGTAGGAGGAAGAAGCAAAGCAGCAAAG
GGCTTTAGGTAGTGGACATACCACCCAGGCATGACAAAAGGGACTCTGTG
GAGCACACACTTCAGGAACTGTTTTTGAACCCCCCCATTTATGACTGCAC
CACTTAAACACAGACAAGAAGCTTTGTTTTGAGAATTAGGACAGTTTATT
GTTTGACCAACATGCTGAGTCTTTTCCACATTTTACACAGTTTAATGTGA
AATCAACATGGCGGCTATGTCTTCTGAGCCCATAACAGATGGAATTGCCA
CCCTCTGTGCTCCTCACAGCCAATCACTTTAAAGGGATGGGTGAGGGGAA
AGTGAGGGGAGAAGTGGACACACACCGCGAGATGCAGGCTGGCCTTCAAT
GCTATGGAGGCTTCCCACCTCCTGAAGGAACCATCTAAACCCCTGCTGCA
AGGATTTCCTGATGAAACCACACACTGCTGGGAGTGCCAACCAGACAGGG
GTCTGGAGTCCAAGGAGTTTGCACATTGAGATCCCAAGGTTTTGGAACAC
CTAAATAGTTCATGTCAAACAAAAATTCAAAGGGTGTCCTGATCTGTGTG
GGTGCCCATGACAATCAATCAGAGTAGACTTGGGGACTGGCCCTTGTGCA
GTAGAGGAGCCCAAAATACCACCAATATTCTCACTCATATGCTGGGAAGA
ACCTAGTGTCCTAACCAAAAAGAGTAGAGATGGTCTGAGGAACACACCCA
CACACAGCAGTCCTTGCTGTGTAATAAATATGGAGTCACATTTGTTCACA
CACAGGGCAGCAATGGATGAAAAATGGAAACTTCACTGGTTCTGATCACT
CCTAACTTGCTGGGTGATCTTAATAAGCAAGTTGCCCTTCTCCATCCTGG
TTTTCCTGCCTGTAGACAGGAAAAGTTGTGACAGCTCTGCTTCACATAGC
TGTAACAAGGTGACAATGCCTCACATTCTTTGGGGAGAGGCAGTTCGGAA
ACGAGTGTGCGCACAATCTGCTCGGTTGATGCACACAGACATCCTTCTGT
TCAGCTGCCCCTCAAGTGGCTCAGACATGCCTCACTGTCCTCGGCTACCT
GGTCCCATCTCATCCTGACATCCTCACCTGCCTTCCATCTTGTGAAGGAA
TGAGGCTGCTCTACCTGCAGGATGCTGAAGCCAGGAGGGCCATCCCTTTA
TTGTCACAACTGGTATCCCAGGGCAGGGCCTCCCCGCACTGGCTAGCCAC
CAGGATGATGCAGACGTTGCACCCCAGATGTGTCTGCACTCTTAACTACC
ACCATTTCAATCCTTGTTTCTGTTGCCAGAGATACTGAAACTGGTCTGGC
CATTGCTCCCCTCAGACCTGGGAACATGACTGAATGTTTAAGGATGACAA
GAAGAGAGCAGCGATAGATAGCAAGGAGGCCTCTGACCAAGGGAAACTGC
TGCCAACAGCTACCAAGCCAACTAGCCTTGGATCACACAACTGCAGCCAT
GGTGCTCATGATTCTGATGTCACACCTTCATGTTGATGTATGTATGATCA
CCCAGCAAGTTAGGAGTGATCAGAACCAATGTGTTTCCACTTGTAAATAG
GCTGTTTGCTTTTTCCATCTTACACTCAACTTTCCTGATGCCTGCAGAAC
AGGAGCTTTGTTGATAAGCTTTTAAGATTCACCCCCTTCACTCCCACCAT
CCCCAAAAAAGAAGGCCAAGAGGATCCTGCTAATCTGCCAGGTCACGTCT
AGTCTTTGGGGAGAGGGACTGAATTGCTCAATTGAGAACATTTCCTCAAA
GGAATCCTCCAATTTGCCCTCTGCTAACCCATCTGGATTCAGCAAAGATG
CAATGCTCTTGCTGGGGTGCTGAGGCAATGCCATGACATCTTCTTCAAGA
GCACCTAACAGTTTATTATGGAACCTTAACTCTGTGCCAATTCTACCTGC
AAGCAAGGGGACAGGGATGGTGCCTGCTCAAGACAAAAATCAGGGAACCA
ACAACTTCTGAAAAAGAGGTCTGGCTGCCTTTCCACACCCCAGGAAGGGA
GGGGCGGAAACCGCACGTGCCGAAGGGAGCGCCATGGAACCACTGAGATG
GGAGGATGGGAGGAAGCCACCCTCAGCCTCAGAGGCCAAGGGGCTGGGGA
GAACCCAAACTCAAAGAGAGGATGTTGTGTGCATGCTAGGAAAGGCAGGA
CTCACTAAGGCCATGTCAAACCAAATGCTATCTTTTACTGCTCCTCTAGG
CCAGGATGGAAACACAGTCCACGGTGCCTTGGATGTGTGTGCCCCTGAGA
AGGGAACTGAGTTTCTACTGTTTTATGGGATGTTTTTTTAAAAAAGCTAA
TCTTTAGTGCCAAGAAAATAAAAATCTACCATACGTTCATTCACTGAGAC
CCAGTGCAGATTCCTGTAAATGGGGCGGTCTCAGAGGCCACAGGTGATGG
CCCCCGCTGCCAACCAGGCAGGACACTGTCATGTGGACTCCTGTAAATTC
TCTCGAGGTAAGTAAGCACTCTTCCAACAAGCACCACTTGGGGGTGTGCC
CATGGTGGGGGGACACTGGCCCACACAGGCATGCACACCCGTGCATATAA
AGTACATAGAGAAATGGTGGAGATATTCCCGTGGGCTATGTTTGCCTGTA
GTTTAACACAACTATGCATTATTTTATTGATGGCAAGACAATCACAAGTT
ATTTGACAATATTAAGTATTTCTTATTTCAACACGTTGCAGTACTTTTGA
ATTTCCAAAACTTATCACAATGCAAGTTCCAACTTGAGAGCAGGAGAAAC
AAGAATATACATTGAGCACATGTATCTCCGTGAGGATGTGTTTGTGGAAG
GTGTATACACAAGTCTATCTCTATATATAATTATGTACACACATATAAAA
ACTAAACACATGACATCTGAAACACGTTCAAACATTGGTCCTTGCCTTCT
GCAAGAGGTGCTCAGTGGGATCCTGCTTCCTCTCTTCTTCACCTGAAGAT
AAATTCCTTGCTAGAGATGAGCTCTGGCATACAGCCTCCTAGGCAGAG

An exemplary DNA sequence is human Trf provided below.

gi|224589820: 73915262-73965820 Homo sapiens chromosome 8, GRCh37.p13
Primary Assembly
(SEQ ID NO: 309)
AAGTGTCTAATCGATTTGCTCAAGGGTAAAGAGAAATTGGTGGAGAAAGAATTCAAACCTAGGCTGGGCA
CGGTGGCTCATGCCTGTAATCCTAGCACTTTGGGAGGCTGAGGCAGGAGGATCACTTGAGGTCAGGAGTT
CGAGACCAGCATGGTCAACACGGTGAAACCCTGTCTCTACTAAAAATACAAAAATTAGCCCGGCGTGGTG
GGGGCCCCTATAATCCCAGCTACTTGGGAGGCCAAGGCAGAAGAATCAGTTGAACCGGGAGGTGGAGTTT
GCAGTGAGCCAAGATGGTGCCACTGCACTCCAATCTGGGCAACAGAACAAGACTAATCAAGAAAGAAAAG
AAAACAAAAGAAAAAAAGAGAAGAGAAAAGAAGAAAGAGAGAGGGAGGGAGAGAGGGAGGGAGACGGGGA
AGGAAAGGAGGGAGGGAGGGAGGAAGGAGGGAAAGAAAGAGAGAAAGAAAGAAAGAAAAGAAAAAAGAAA
GAAAGAGAAAGCGAGAAAGAAAGAAAGAAAAATTCATATCTAGAACTCTGACTCCAGAGCCTGAAATCCT
AACTGCATGGGATTGTGCTGTCCAGTATGATAGCTACCAGTCATACTTGGCTATTTGAGCACCTGAAATG
TGGCTACTCCAAACTGAGATGTGTGTAAGTGTAAGATACACACAGGATTTTGAAAACTGTTTTTTTAAGT
AAAATATCCAAGTTTTAAAATTACATGTTGAAATAATAGTTGGAATATATTTATAAGATTAAAATTAACT
TCACCTGTTTCTTTCTATGCTTTTTAATGTGGCTCCTAGAAAATTAAAAATTACATATATGTGGTTCGTA
TGCTGCTTCAAAAAGTAGAGGCTGTTCATGGTTTTCTTTCTTAGGGCTGTCAAGGTCAAGGCGCTTCCTG
TTATTTCTCTATTCAAGGGTAGAAAACATCACCAGGGTACGTTGTTTTATTGGGCATTGGTTTATTTCTA
GGGGGTGGAAACGGGATTCTTCCCCTAGGTCCCCAGCTGTTTGTAGTCATATTCTTTTACTCACACACAA
TTTTATTGCTTTTGCTTTTGGGAGATGTGTTTTATTAAAAATAGTGGCTAATCAGGGACTTGTTAACGGC
ATACACTAGAAGGTTTTTTGTCTGTTTTTTGTTTTTTTGAGACGGAGTTTCACTCTGGTTGCCCAGGCTG
GAGTGCAATGGTGCAATCTCGGCTCACCTCAACCTCCGCCTCCTGGGTTCAAGCGATTCTCCTGCCTCAG
CCTCCCGGGTAGCTGGGATTACAGACATGCGCCACCACGCCTGGCTAATTTTGTATTTTTAGTAAAGACA
GAGTTTCTCCATGTTGGTCAGGCTGGTCTCGAACTCCCGACCTCAGGCGATCCGCCCGCCTTGGCCTCTC
AAACCGCTGGGATTACAGGCATGAGCTACCGCACTCTGCCTTGTTTTTTTTTTTTTTTTTTTTTAACAAA
ACGATCAAACAAACTGACCATGGAAAGTCAGTCCTGGGTTCCAGTTTTCAACTAACTGCATTTTCTTGCA
GAGTGAGCCCGCACCATCACAGTGCCTGTATTCTGTATTCTGTCAGACCCAGGCTACACACGGATCCTGT
AATGCATAAAACAACTTAAAAAAGTCTCAAGAGGCTACAAAGATCTTTTAAATACGTTAAAAGCTCTAAA
CGATTAGAACTGCCTAAGATAATGTTTCTCTTCCTGAAACCTGCTTTGCTTTAAACTCAAAACATTCCTT
CAGTCCTTGAGCATTACCTTATTGATTCTGAAAAGGGAGGAAGTCTTCATGAGTAGGAGTGTGCTTCTTT
ATCATTATTATATACTATGTCCACTACCTCGAAATGGAGTGGTTCATTTTATTTGTATGGTTTGGTAGCT
AGCGACCCTGGTTCTATACTGGAATCGCCTAGAGATACTTTGAAAAACACATATGCTTGAGCCTTGCCCT
TAGAAATTCTGATTTAGTGAATACAAAGTATAACTCAGCCAACTGAGAACATACGACGTGCGGTCGGGGT
TGAGAACTATTTATGAGGATAGCGAAACTAGTGTTTTAGGCCCTGTAAAATTTACTTTAAAAAAAAATCA
GTTCATACCCAGGTACTTCCCCTCCGCATCTCTGTTCAAACCTTTTTAATGAAATCCCACTGGCTTAGAC
ACTCTCCCTATGAAACCGCCTTTGCAAAAATCATAACTGAGAAATTACAGTGAAAGAGGTCTGACCTAAC
CGATACTACCTTTCTTCTAACATTCAAGCTGTCCATTCCTAGGTGTAGGCCAAACTAACTTTGGGAGGAA
CTTAGTTTATAGTTTAGCTTGGAAACAAAGAATATAACGGTGCTTTCCCCCAAACAAACCCCCTTCCTGC
CTGGGGACTAGACTGCCTTTGCAGGACTAACAAATTAGCTACAAGATTAGAAGTTATGGTTTAGCAGTCA
TGAAACTGGAGGCTGCAAGATTGTAAACCTCCCCAAATGGCTCCTGGGAATAAAATCACTATTGTAAAAC
CTAAGATCAGTGCTTGAGATATTTTGCAGACCCTGCACTCAATGCATCAGCTGGCACCACCCAGATCAAT
AAACAGGCTCATCTAGTCTTGTGGCCTCCACCCAGAAAGTGACTCAGTGCAAGAGGACAGTTTTGACTCC
TTACGATTTCATCTCTGACCCGACCAATTATCACTCCTGACTCAGTGGTCCCCTACCCACCAAATTATCC
TTAAAAACACCAATCCTCGAGTTTTCGGGGAGATTGATTTGAGTAATAATAAAACTCTGGTCTCCCACAC
AGCCGGGTCTGCATGAATTACTCTTTCTCTATTGCGATTCCCCTGTCTTGATAAATCAGCCTGTCTAGGC
AGTGAGCAAGGTGAACCCGTTGGGTGGTTGCACCCCCACCAAACTCCTATTAAGGTTACTGTTAGCATCA
CTCATTAGTCTGATGTTTATGTACAATGTGTGTGTGTGTTGCTTGTCTTACCAACTGGTCTATAATTCTT
TATGGAGAGGAGTAGTATCTTTATATTTACCATGCACCATCCTAGCGCAGTACATTTCACATAATAGGTA
CTCAATAAAGGATGTTTCAAAAGTACATATGTAGTTTGGATATTCACGAGACATTTATAAATGCAAAAAT
AAAGCAATGTATATAATTCATAATGAGACCATTAGCTTATATTAGCAATGGGATAGACTAAAATATGGGT
TAAAACACAATTATTTAATATGGTAGACATAAGAATTTAGGAAACTTATAGATTTCAAAGTGTTGTTTAG
GAAATAGTCCAAATCAAAAACAGTCTTGGGAAATGCAACAGCATGGGGAATGTCATCTAGGCTTTATGGG
AATCTTTCCACAGTATCTCTGACTACTGGCATCCAGAATCATAGATGTATACTTACCTGGCAGGGGAGAT
ACCATGATCACAGGATCATTAATGATTCCAGTGGTGAGAAATCAGTACCTGGCAGTAGAAATCGGCACTT
TATTGTGGGAAAGCCTGAATGGTTAGGAACCATGTGGGATATGAAGTCAAATCTGCCACCCCAAACCTTC
AAACTTTGGTTTCAGGTTGTGCTCTTAGGATCATCACAAAACAGATTGAATCTCTCCCCTCATAACAGTC
CCATTAAATGAGGTAAAGTCAACTATACTTGCGAGTCTTACTCCATGAACTCATGCAAGAAACATAATTT
TACTCAAGGGATGATTTTTAAATCACCAAAATCACACAGGATTCTCTTTCCTAACCCACTCCAGCACAAA
GGCACTACCTCCCAATACCATCCCATTGGGGGTTAGGGCTTCGGCATGTAAATCTGGGGAGAAAACAAAT
ATGCAGTCCATAATACTAGGTTCATAATGTGCTGTAACCCTTATTCCCTTAGCCTCTATCCCCATTCCAC
TGTATTATTTCAAAACAGACCTTACTTCCCATGGGACAATTAGAGCCTGAAGAAGAGATCTCTTCTGATG
CATCATCATTTTTCCTGGCAGCACAAATATTCTGCTCTTCCTGCTAATACTAATTGAGCACTTACTATTT
CCCTGGCTTTTTTTTTTTTTTTTTTCGGTAGAGATGAGATCTCACTATGTTGCCCAGGCTGGCCTCAAAC
TCCTGGGCTCCAGTAATCCTCCCACCTTGGCATCCCAAAGTGCTGGGATTACAAAGTGAGCCACTGTGCC
TGGTGAGGTTTCGTTTTAAACTCTTTTTTTTTTTTTTTTTGAGACGGAGTTTCGCTCTTGTTGCCCAGGC
TGGAGTGCAGTGGCCCGATCTCGGCTCACCGCAATCTCCGCCTCCCGGGTTCAAGCAATTATCCTGCCTC
AGCCTCCCGAGTAGCTGGGATCACAGGCATGCACCACCACACCCGCCTAATTTTGTATTTTTAGTAGAGA
CGAGGTTTCTCCATGTTGAGGCTGGTCTCGAACTCCTGACCTCAGGTGATCCGCCCGCCTCAGCCTCCCA
AAGTGCTGGGATTACAGGTGTGAGCCACTGCGCCCGGCTGTTTTAAACTCTTTACATTTACCAAGCCCTT
TAATCGTGCAGGTATTATTATTATTCACATTATACGGACAAGAAAACTGACACGTAATTTACCCAAGGTC
ACACAGCTAGTGAGTAACTGGTAGGGAATATTCCAACCTAAGCATTCTTAATACTAGAATACAGTGCCTT
AGTTACTCAACACTTAAACTGTACCACCTTAAAGACAATAACAAGAAGTGTGTGAAAATGTGGAAAGGCT
TTGATGATGCTGGATGCGTTATAAACAGAAAGGCTTTTAAAATAAACTTTTTATTCAATTATGACACACA
TACTGAAAAGTGCATAAACGATGTTCAGTGAATTTTCATAAAGTGAACCCACATCAAGGAACAGAAAACG
GAGCCAGGCGTTGTGGCTCAGGCCTGTAAACCCAGCACTTTGGCAGGAGGATAACTTGAAGCCAGGAGTT
GGAGACCAGCCTGAGCAACAGAGCGAAATCCTGTCTCAAAGGAAAAAAAAAAAACAAACAAAAAACAAAA
ACAATAACCAGACCACCCATCCCCATTTAGAATTTCTTTTTGTGCTAAGTTTCAAAATTCAAAATAAGCA
TTCACTTGTTTTCTACAAACTGCATTCTATAGTCGATCTCAAACCTTGATTGAGATACATTTCCATTATA
GTATACTCTTTCTAGAGCACTGATCTCAAAATGTGGTGCCCAGCCAAGTAGCGTTGGACTTTCAAATTCA
AGGATCCCAAGCTAGACCAGCAGAACAGGAATTCTGGGAGTGGAGCCCGGCAATTTGTGTTTTAGCAAGC
CCCCAGGCTGATTCTGATGCATCTTAAGGTTGGAGAAGCATGGATATTGTCAGTTGTAATAGTTGTTGTT
CGTGATCACGTAGTAACTGTTAGATGAAAACTAGGGTTTAAAAGTGTTACGCTTGTCTAATAGTGACAAG
TTCTCATTACCCATCTCTTTCTGAAATAGGCTGCAGGCAAATGCGTTTTCATTTTAAATAACCGTGTTGT
TGTTCCAGTCAAGGTTGCATTTTACAAGGAATAAGGAAGATAAAGCTGCCTCCTCCTATCCTAATCTCGC
TCCTCGCTCAGTATGGAGTCGTCCGGTTTACCTACAAAGCCCTGCCCTAGACTGGCGGCAAATTCAACGC
CCAGAGGCAGGAACAGGTGTCGGCCAATCAGAAGCCGGAACAGGTGTCGGCCAATCAGAAGCCAGGCTTG
AACCGCTCGCCCATGTCCCGCCGGCGGAAGAGACTCTCCGCGGCCACGCCCCGAGCCCTCGAATGCGAGC
CAATCGTTGCTCGGCGCCTGAAGGGGCAGTACCCAAGCGAGCCATTTAACATGGCGGAGGATGTTTCCTC
AGCGGCCCCGAGCCCGCGGGGCTGTGCGGATGGTAGGGATGCCGACCCTACTGAGGAGCAGATGGCAGAA
ACAGAGAGAAACGACGAGGAGCAGTTCGAATGCCAGGAACTGCTCGAGTGCCAGGTGCAGGTGGGGGCCC
CCGAGGAGGAGGAGGAGGAGGAGGAGGACGCGGGCCTGGTGGCCGAGGCCGAGGCCGTGGCTGCCGGCTG
GATGCTCGATTTCCTCTGCCTCTCTCTTTGCCGAGCTTTCCGCGACGGCCGCTCCGAGGACTTCCGCAGG
ACCCGCAACAGCGCAGAGGGTGAGTGCAGACCGCGTCCGGGCCGGGACTACGCGGGGGGCGGATGCGGGC
TCCGTGGTGCGCGGCAGAGGGCTGGTTCCCTACGTCGCCGAGGGAGGGGCTGCTGCGGCCGATTAGCTGG
GAGTCCGAGGCTCCGGGCTGAACTTTGTTCGAATCCCGGTTCGCCCGTTGTCAGCACCTGAGTTTTTCTC
AGATTGATTTCCTCTTCGATAAAATGAAAATAAGACCCTGTTTAGGTTTGATGCAAGCATTAAATGAGTT
AAAATGTATACAGTTCCACTCAACCCAGTACCTTTGTCTCTTTTGGCGCTTTCTACTCAGGTATTCCTGT
AGTAATATTCAGCAGTCCCTGTGGCCTCCAGTAACCCCCGAGGCATCAGTGTTTTATGTTAATTTGTGAG
CTATTAAATCTCATTTGGCCTGCTGGCTCTACAGCAGTTACTTTTATTCTGTGTAGTCTCAGAATAAAAG
TAAATGTTGGCCACATGCCTGCAAGTCCCAGCTACTTGGGAGCCTGAGGTGGGAGGATCGCTTGAACCCA
AGAGGCAGAGGTTGCAGTGAGTCGAGATCGTGCCACTACAGTCCTGCCTGGGTAACAGAGTGATACCCTG
TATGAATAAATAAATGAATGAATGAAACTGTTCATGAAATCTGCTGCACCCCGCCCCCGTCCCAAGATGG
GGTCTCACTGTCACCCAGGCTGGAGAGTGTAGTGGCGCCATCACAGCTCACTGCAGGCTCGACCTCCTCA
GGCTCAGGTATCCTCCCGCTTCAGCTTCTGGAGTAGCTGGGTCTGCAGGCGTGCACCACCACCCGCAGCT
GTAAGATTACAGGTATGAGCCACCGCGCCTGGCCTAGTGTAGCTTTTTGAAAAACAACAGAAAGATGTTC
TGGTACTTCATTCATAGTTGGCGCTAAAATTACTGTGCAAGTATATATTTATAATACTATAATTATGCTA
TTTAATAGAATTCTTAGACATTTTGACACATTTCATCTTCACCATAATCCTGTGAGTTAGGCAAATAATC
TTAGTTTTAAGGTACTGATAGGTTAAAAGGGAGTGTTTCAGCCCCATCCTTTATCTTCCAGTTCAAGGAT
CTTTCCTCTGTATTGCGGTTAACACTGGAGCCAGGGATAATTAACATTAAGTAGAAAATTAAATCCTGGA
ATGAAGCTAGTTCCTCAGAATTCTCTTAGAGTTCTCTTAGAGTTGGAGACATCAGTATGAACTGTTTAGC
TCATGTAGGTATAGATGACTTAGAAATATTTGCATATGCATGTGTAGACATAGGTTACTATACAAATATG
TATTTCCTTACTCTGTCAGATAAAAGGGCACAGAAGCAACCTAACTCTAGTAACAATGAGTACACCTAGC
ACCCCGATCTTGTTTACAACTAAGTTTAGTTTAAAGACCTCACTTGGCTTTATTGTGATTCTGGATTCAG
GTAATACTTTATTCCATAAAATAAGTGCTCAATGAATTGAGCAGTTGGGGGTTGGCTTTATAGACAGTAA
GGACTTAAGGAAGCTGAAACAGAACAAAAAGAAGATTGGTTATTTCACAGTTACTTGTAAGGCTGGGACA
GGGAGACAGAACAATAGATACATGGGATGCAGAATGGATGTTGTGTTATCAGGCATGAAAGCAACAGTAA
TTTCCTTGTATGTACGTCTCCCTCAGAGCTCTTGAGTGGTTAGGCGCATTATCAATGAGCAATGGTATTT
TGAAAGGAATCTTTTTTCCAAGCAGTAGATCTCAATAATGGGCTTAAAATATTCAGTAAACCATGGTGTA
AACAGAGGCACTGTCATCTTGTTCCATTTCTAGAGCACAGGCAAAGTTGATATATCATTTGAAGGGCCTT
AAGATTTTCAGAATGAGAAATGGGCAATGGGTTTAACTTAAAGTCACCAGCAGCATTAGTCCCTAAGGAG
AGTCAGCCTGTCCTTTGAAATATTAAAGTTAGGCATTGACTTCTCTCTGGCTATGAAAGTTCTCTATGAC
ATCTTCTTCTAATATGAGGCTGTTTCATCTACAATGAAAATCTGTTGTTGGCTGGATACAGTGGCTCACA
CCTGTAATCCCAGCCCTTTGGAAGGCCAAGGCAGTTGGATCACTTGAGGTCAGGAGTTTGAGACACCAGC
CTGGCCAACATGGCGAAACCCCATCTCTACTAAAAATACAAAAATTAGCTGGGCATTGTGGCACACATCT
GTAATCCCAGCTACTCAGGAGGCTGAGGCACGAGAATCAATCACTTGAGCCTGGGAGGTGGAGGCTGCAG
TGAGCTGAGGTCACACCACTGCCCTTCAACCTGGGTGACACGGCGAGACTCTGTCTCACAAAAAAACAAA
ATCAGTTGTTTAGTGTAACCACCTTCATTTGTGATTGTAGCTAGATCTTCTCAATAACTTGTAGCTTTTA
CTTCAGCACTTGCTGCTTCACCTTGCACTTCTATGTCACAGAGATGGCTTCTTTTCTTAAACCTCATGAA
CCAACAACCTCTGCTAGCTTCCAACTTTTCTTTTGCAATTTTCTTACCTCTGTCAGCCTTCTTAGACTTG
CTCTGGATTAGGCTGTGGCTTAAAGGAATGTTGTGGCTGGTTTGATTTTTTTTTTTCTATCCAAGTCAAA
CTTTCTCCATATGAGCAATAGGCTCTTTTACTTTCTTATCATTTATGTGTTCAGTGGATTAGAACTTTTA
ATTTCCTTTGAGAACTTTTCCTTTGTATTAACAACTTGGCTGTTAGACACAAGAGGCCTATGTTTCAGCC
TATCATGGCATTTGACATCCTTTCCTCACTAAGCTTAATCATTTCTAGCTTTTGATTTAAAGTGAATGAC
TTGCAACTCTTCCTTTCACTGAAACACTTGGAGGGCATGTAGAGTTACTCATTGGCCTAATTTCAATCTT
GTTGTGTTTCAGGAGATAAGGAGGCCTGAAGAGAGGGAGTAAGGGGAAGGGCTGGTCAGTGGAGCAGTTA
GAACACACACAATATTTATGGAATAAGTTTGTGTCTTATGGGCATGATTTGTGGTACTCCAGAACCTTTA
CAATAGTAACATCAGAGATTATTGATCACAGACCACCATACAGATATAATAATAATGAAAAAGTTTGAAA
TATTGCAAGAATTATCAAAATGTGACAGGTACAGGTACTGTTAGAAAAATGAGCCAGACGCGGTGGCTCA
TGCCTGTAATCCCAGCACTTTGGGAGGCTGAGGCGGGCAGATCACGAGGTCAGGAGATCGAGACCATCCT
GGCTAACATGGTGAAACCCCGTCTCTACTAAAAAAATACAAAAAAAATTAGCCAGCCTTGGTGGTGGGCG
CCTGTAGTCCCAGCTATGCGGGAGGCTGAGGCAGAAGAATGGCATGAACCTGGGAGGCGGAGCTTGCAGT
GAGTGGAGATGCACCATTGCACTCCAGCCTGGGCGACAGAGCGTGACTCTGTCTCAAAAAAAAAAAAAGG
TGCCGTTAGACTTGGCTCAAGCTGGGCTGCGGTAAACTTTCAATTTGTAAAAAACATGCAATCTGCAAAG
CACAATAAAATGAGGTATGTCTGCACCTATCCTTCGTGGTACTCATTAAAATTTTAATTAGTTACTTACA
TAACTTTTTGTTTAATTGTGTCTTCTCAGATTAAAACCTTCATGGGGGCAGAGACTGGCTATCTGAAGTC
ATCACCTCCTAACACAGGCTGGCAGTAGATGTTCAGTAAATATTGAATGGCTTGTGTTTAAATTCTATAT
AAATTTTGTGCTCTCTTAAAATCCTGTTAAATTCTATTATGACTGTGTATGGTCCAGATAATCTCTGGTG
TAGCATAAGATTATGTTAGAATTGAGGGAGGAAATTGGGTAAAAATATATTCGTCTTATATGCTTAATTG
CTACAGAATTGCTTTTCAGTTTGCTAGTTATTTAAGTTTTTTATATATACACCAACCAAGCCCTGGGTTA
GGTATAATGGTAGTAGTGGCACCTTCAGGTCTTAGGTTAGACATTATTTCCTCAGAGAAGCCTTCCCTGA
CACCTCCTCTGTGCAGAAGATACTTAAACACACAGATTTCTTGTTATATGCTTCCAAAGCATTCTGTACT
TGTCTTTTTAAAGTTTTCACAACATTTTTACTTCAGTAGTTCAATCGCTTGTCTCATTGCTACATGGTAA
AAAGCTCACTGTGGTCCAGGAGTATGGCTTACTTGGTATTGTATGTAATCACTGCCTGACTCAACATCTG
GGTGGGCATTCAAGTTTGGGGAGGTTAATATTAATAGGTTTTAAACTTTGTAAGCTAAACAGACTCACTT
AACTGAAACCTAGATAGAAATAATATAAAATATTTTCTATTATATGAAATGTTTCTAGGTATAAGTTTCC
AACCAATGATTTTACACATTTTACAATTTGTATCTAAATCTTTTTTGTTAATGTAGACTGCTACTTGTGA
AAGAATTTTCAACAATTTTTATTCTACATGTGAAAGTTATATTTTGTAAGAAATGTCTTCATTGCTATTC
TTTTTTTTTGTTCTGAATTTTAATCAGATCTTAAATGTACAACTCTAAATTTATGATAATTTTTACAATA
TGTTTGTAGCCTATTACTCAACAAACCACACAGTGGCACTACTGGATCAAGTTTGATTTTAATAAAATTT
ACTTTTTTTCTTTTTTAGCTATTATTCATGGACTATCCAGTCTAACAGCTTGCCAGTTGAGAACGATATA
CATATGTCAGTTTTTGACAAGAATTGCAGCAGGAAAAACCCTTGGTAAATATGTTTTTTTATTTTATATT
GGAAATATTTCAATCTGTTTCTAATGTAAGACAATGGGAAGAAACAGACGTTTTTGGGGGAAGTTTGCCT
ACAAGTGTCTGGTGCATAGGGGTGTGGGGGGGTGGTGTGTGTGTGTGTGTACACATACAGTCATATCTAA
AGCAAGTAAAAGAAATGTAGTCCCTGTCATCTTAGAACATTGGTTCTCAAACTTTAGCCTGCTCCAGAAT
TACTTAAAGGGCTTATTAAAAAAAAAAAAACATAGTGCTGAGTCTCCCTAACTCTCATGAGTTTCAGATT
CAGTAGGATGGGTATGGGGCTCTGAAATATACATTTCTAATAAATTCCCAGGTGATTCTGATGCTACTCT
TGGGGAAGTGTGTTTCATTGACTCTATTTGCTAACTTTGTGGTGACTCCAATCATACACACTCTGTTTTT
ACTGGCAACTATTATATTTGAAACCTTTTATTTGAACTTGATGCCAGCGCTTACGTAAGAAGTACAGTTT
ATAGCTGTAATCTCTCTCCCTTGTCTCTGTAGTTCCATTTTAGATTGTAATGGGCAAGGCTGATTCAGTT
TATCAGTAATGGCTGGTATTTACAACATGGTCATTTTAAAAGGCTATTTGATGGAAATAATTTTGTATTT
AGATTGGTTTTTATAATTTACAAAATACTTTTCCAACAATCCTGAGTAGTGGGCAGTACTTACATAAAAC
TGCTTGCTTATTGAAGTGTTGTCTTACATAAAATCCCAGTGTATTAGCACCACTGTCAAGGCCTTCACTA
AAGTATCTCTGGCTGATGTAGTCTGCTTTGTTTGGGGGGTGAGGGTAGGTCTGGAGTTGGTGCCAGGACC
ATAAATGGATAGAACTAGATAGAGGCAGAATAGTTGTGAAAGAGACAGAGCCATAGGTAGGCCTGGGCCA
GTTGCTGGAGAAGTAGCAGGCTGGAGCAGAATTCCAGACCATGCAGACTATAAGTTGACACTGTTGTTAA
CAGGGTTGTTTTCTACATTAAGGTGGAAAAGTATTCTAAAGTGCCTGGCAGTGCTTTACACATAGCAGTT
GTTCAGCACATCTTAGTTGCTTCCTGGATAACCTTTTCTCCAAAAAAGGCAGGGGGGAATCCAGATTTTA
TGTGTGATCTCATGGTTTAATCTTGGAATTTACCAGGATACTCTTTTGGATATATCCTAGATATAATAGG
CCAGGGTCAGCTGTCAAGAATCAATGTATTTCACCATAATGCACTAAACTGAGGCAGCATTTAAAATTTT
ATGTATTGGATTTTTGGCTATACATAACCCTTTTGCTTTTTTTTTTTTTTTTTTAATTTTGCTCTATAGT
GGCACTGCCTAATAGAGATATGTGAGCCACATGTCTAATTTAAAATTTTCTAATAGCTATGTTAAGAAAA
GTAGGCCAGGCATAGTGACTCACACCTGTAATCCCAGTACTTTGGGAGGCCTAGACAGGAGGATCGCTTG
AGCCTAAGAATTTGACACCAGCCTAGGCAACATAGCGAGACCCTGTCTCTACAAAAAATTTAAAAAATTG
GCTGGGCGTGGTGATTCACGCCTGTAATCCCAGCACTTTGGGAGGCCGAGGTGAGCGGATCACGAGGCCA
GGCAATAAAGACCATCCTTGCTAACGTGGTGAAACCCCGTCTCTACTAAAAATACAAAAAAAAATAAAAA
TAAAAAGTTAACCACGTGTGGTCCTCTGGAGACTGAGGTAGGATCGTTTGAGCCCAGGAGGTTGAGGCTG
TGGTTAGCTGTGATTGTGCCACTTCACTTCAGCCTGAGTGACAGAATGAAATCCTGTCATGAAATTTAGA
AATTCCCATTCCAGCAGGTGTAGCTAAAAAATAATAATGATAATTAAAATAGGTGGAATTAATTTTGATA
ATCTTATTTAACTCAATATTTCTCAAACACTATGTCAACAAGTAGTCAGTGTAAAAAATGTTAGTAAGGC
CAGGCACATGGCTCACACCTGTAATCCCAGCTCTGGGAGGCTGAGGTGGGTGGATCACTTGAGCCCACGA
GTTTAAGGCTGCAGTGAGCTATGATCACACCACTGCACCCCAACCTGGGCAGCACAGCAAGGCCCCCATC
TCTTAAAAAAAATCAAATGAGATATTTTACCTTTTTTTGGAGTAAGTCTTTGAAATCTCATGTGTACTTT
ATACTTAAGGTGCATCTCAATTCAAACTAGCCATATTTTAGCTGTGCAGTAATCATGTATGGCTAGTAGC
TGTGCAGTAATCATGTATGGCTAGTAGCTACCATATTGGGTTTAGTCTCCTACCCCACATAGGTTTGACA
AGATTGGACATGTTTGGGGTGATGTGGCTGTAGACCCCCACGTAGCTCTGAAATACAGCAGATATCTTAA
AGGGGAAATGCTTGTCATCCTCCTCTTATGGAACTTTAAGCTTTTTTTTTTTTTTTTAGCCAGGGTGTCT
CTCTGGCCCAGGCTGGAATGCAGTGGTGCCATCATAGTTTATTGCAGACTCAAACTCTTGAGCTCAAGTG
ATCCTCCTGCCTCAGCCTCCTGAGTAGCTAGGACTACAGGTGCACCATCAGGCCCAGTTAATTTTTTAAT
TTTTTGTAGAGACGAGGTCTCATGTTGTCCAGGCTGGTCTTGAACTCCTTGCCTTAAGTGATCCTCCCGC
CTAGGCCTCCCAAAACTCTGGGAGACTTAATGTGCCTTTCCACTGCAGCCTATTGCATTGCCGTGGAACT
CAGCACATGTCCTGTGGAGAAAAGCCAATCATGTTTGCAGTTTCTCTAGTTTCCAGTCGACGTGCCAGCC
CCAAGGAAACCACCAGAAGCTGTTCTTATTTGTCTTTCTCCAGTAAAGTCTCTCTGGCTGGAGTGCACTG
GATCCTCAGCCTTATCCAAAATCAGCAAATCGGCAGCTCACCTCAGAAGGGATTCTCTCTGGAATTTTAG
TTCATCTCATTTTCATTGCTTCCATGGCTATCCCATGTCTTTAGCAATGTGATTTTGGAGTTTATCTAAC
TGTTTTCTAGCTGTAGTGTGAGTGTTTTGTTTGCTGCAGTATACTACATCCCACCTGGAAGTGAATGTCT
ATACTGTAAATTTTTTGAAGCTAAGATCTAACATACATTTTTCTCTATGTCACATTTATAAGAAGGAAGG
CAGATAAGTGAGAAGGGTTAAGTAATGTCAGTCAAGAAATACTTATTTGAAAATGTGTGTTGAAATTAGC
TCTTGAAAGAAAGCAAGCAGATGAAAAATGAGGAAGAAAAATTAGGCAGTCTACCTTTGCATTTTCATTT
CACTACTTTTGAGGCACAGTAAGTGATATTTAAGTGTCTTGGTGTCTGCTTGTACTACTTGCTTGGTGTG
TGACCTCTCTAGCTCTCAGATCTTACTGTATAAAACAAGTGCCACAGTGCCTGACACTCAGTGTTAGTTA
CTAGCATGAATATCTTTTGTACACCTCTTTCTTACCTTATGCTGCTCCATTTTCCTGAAGTGTTCTCCTT
CCCATCCTTGTAGTCTACACTTAGTTATCATCTGTTTATCCTTAATGCCCCAATTTGGATGACTTCTGTT
TCATGAGGGATTCACTTCTGCTATTCCTACTTAGAGTTAATACCATGTTCCTCAAATACACTAGACTCTG
CTTGAGAATTTAATCTCTGGGAGATGGGTATAGGCATCAATATTTTTTTTCTTTTTTTTTTTTTTGAGAC
AGAGTCTCACTCTGTCACCAGGCTGGAGTACAGTGACGCCATCTTGGCTCACTGCAACCTCCGCCTCCCG
GGTTCAAGCGATTCTTCTGCCTCAGCCTCATGAGTAGCTGGGACTACAGGCGTGCGCCACCATGCCTGAC
TAATTTTTCTATTATTAGTAGAGACAGGGTTTCACCATGTTGGCCAGGATGGTCTCAATTTCTTGATTGG
CGGATCTCGTGATCCGCCTGCCTTGGCCTCCCAAAGTGCTGGGATTACAGGCGTGAGCCACCGCGCCTGG
CCATGCATCAATATTTTTCAAAAGCTCTTCAGATGTTTCTGAGGTATAGCAAACATTGAGAACCATTGGC
ATAATGCACAATTCCATTAAAGTACCAGTTATATTGGTTTGTCTGCATGTCTTCTTCCATAGCTAGGATG
TGAGTTCCACAAGGTCAGGAACTTTTTTTTTCATGTTTGTATCTTAAGTACCTGACACAGAACCTGGAAT
ATAAAGAGACTTAGTATGTGAATGAATATATTTTTATATCTAAAATGTTGGAAAAACAATTTGGAGAGGA
CAACAGGAAATAAAGTAAAAATATTTGAAACTACGAATATTTAAACTGCATGCTCACTCTGGTTTTTCAA
GGAAAAAAGGATGTGCTATTTAAAATTCTCTTTATTCGGTCGGGCTTGGTGGCTCACGCCTGTAATCCCA
GCACTTTGGGAGGCTGAGGCGGGTGGATCACCTGAGGTCAGGAGTTCGAGACCAGCCTGGCCAACATGGC
AAAACCCCGTCTCTACTAAAAGTACAAAAATTAGCCTGGCGTGGTGGTGGGCACCTGTAATCCCAGCTAC
TTGGGAGACTGAAACAGCAGAATCGCTTGAACCCAGGGGGCGGAGGTTGCAGTAAGCCGAGATCGCGTCA
CTTCACTCCAGCCTGGTCAAAAGAGCAAAGCACTGCCTCAAAAAATAAATAAGTAAAAATAAAAATAAAA
AATTCTCTTTATTATGCTTATTTGTTGCATACTTGCTATGGAACTACAACTGTGCTAGGAATTACAGGGA
TTTAGAAAAATACACTTAAGTGGCTTCTGCCCCCCAAAATTTGAAATATGCATTATAGAGTAAGAAGTTT
TTTGTCTTCAAGACACTGTAAAAGCTAATAGGGTTGTTGAGGTAAGTATAAAGTACTGCAAGGCATTAAA
AAATGAATAATGATACAAGAGTTTCTTTTTAATTAATGCAGTGCTCATGAATTTTTGTGTCATCCTTCTC
GAGGGGCCATGCTAATCTCTGCATTCTTCCAATTTTTATATATTTGCTGCCAAAGCAAGCACATACAGGA
GATTTAGAGAAAGGACATTTCCTAGTCTCTTAGCTGCTGGGGTAAGGACAGCATTCAAGAAAGAGGTGCT
GTTTGCACTATGCCTGTAAGAATTGCTAGATTTTGTCAGAAAGAAAGTATAAAAATGGTAGTCTATCCAC
ATCCTCAGATGTGGTGTGGGGGTGGGGGGAAGGTAGTCTAGATAAAATGAAAACATTAGCATTATTTTGA
AAATAAACTATAGTTTACCTGGATAATAGACTCTTGATTTCCATATACAACTTCATAAACTTGGTCCTCA
CCCAGGCTTCCTATGATAGTAAATGTTAGCACCATTTATCCAATTATTAAAACCCAAGAGTCCTCCATTT
TTTCCTCATGCTCTACATGCAGTCCATTAGCAGGTTCTATTCTACATTCAAAATAAGTTCCAAAACAGAC
AACTACTTGCCTTTTCCATCATCTTTCTAAGAGTATGCATCCAACTTGCCCACCGACCTTCCTCCTGCCC
TTTGTATTTGTTTTTTCAAGACAAGGTCACGCTGGAAGGCTGTGGTGTCATCATCGCTCACTGAGCCTCA
GACTCCTGGCTCAAGCAGTCCTCCCACCTCAGCCTCCCAAGTAGCTGAGACTACAGGCACACGCCCCCAC
CTGGCTCTTCCTGCCCTTTGAACATCAAACTCCTCCTCAGGACCTTTGCACTTGCCATTCCCTCTGGAAT
GTTTTTCTTCCAGATCTTTGTGTGACTGTTCCTTCTGCTTCACATTACGCTGTTCTCAGCTCCACTGTCA
CTTCTTCAGAGAGGTCTTTAACCTGTCTGAAAGAGCTTCCCTCTCCCCTACCATTCTTCATTTCCAGAAC
CTTTTTTCTTTCTTCTAGCCCTTACTACTCTGAAACTATATTGTTTACTACTCTGAAACTGTATTGTTTA
CTACTCTGAAACTGTATTTATTTCTCATTAGCATGTGCGTATCATAAGAGCAGGATTTTGTCTTACTGAG
CCATTCCAAGTTTTTAGGCGAGTGCCTATATATACTGAGTGATCAATAAATATTTATTGAATGAATTGTA
GACTGTAGATGCTAGACATGTAAGACATTATCATGAAGGCTCTTGAATGTCCGAATGGGTTGTTGCTGTG
TAATTTGCTAATTAGTTATCAGCTGTTGTGAGTCTTTGTTCTGAATAACTTGATCACAACTCTTTTAGGA
AAGCTTAATAATAGTACAACTATTATCTCATTATATATTCACATAAATTCTGTGAGATAGTATTTATTCT
CACCATTTTATAGGTGAGAAAACTGGCCTGTCTTGAGGTTTGTTATAATGCATTAGACTTAGGAACCACT
TAAGAATTCTGTTGCTATGACATGTTATGTAAAATAGTTTACTTTCGTTCTTAAAATTCATGTTTTTGTA
AAGAAAATCTTCAAATGTTTGTAATCTCATCAACCTTTTTCTATTTGAAATTGATCTTAATGAGCAAATT
AACTTAGAAAATAAAATCACTTCTAAACTGGTATAAACAAGAAGAGTACCTAGCACATAGTATACACTCA
GTAAATATTTAAAGGAATGCATTATTAAAAACTGACAAAGTTCTACTTAGCTTTCTGAGTTACTTATTTT
TGTTCTGTTTTTAAAAGATGCACAGTTTGAAAATGATGAACGAATTACACCCTTGGAATCAGCCCTGATG
ATTTGGGGTTCAATTGAAAAGGAACATGACAAACTTCATGAAGAAATACAGAATTTAATTAAAATTCAGG
TATGAATAAATTACTTTTATGCTTATTATATTTCTAGAAAATAACATTTAAAAGAAATTAAGAGGAACCT
AGTAGAAGTTGAGCAGTAGTAAAATTTAATGTATTGTTTCTCTTCAGAAATTTGACCTTTGGTTTATAGT
CTTAACATGAGCACTGGGGGCTGGAACTTAATGTGCCATACTTAAGCATATGTTGCCTCTATGTTGTGGA
AAATTTAAAAACCAGGGGTTTGTGCACTCTCAATTGATGCTTAAAGATTATGTAAAAAGTAGATACTATT
TTTAATTAATGGTAAATTTTAGATTTTTGTAATCAAGAATATATAAAATTTTAACCAAGTAGGGAGAATT
TTAGTAGACAGTAAGTAGCCCAAAACGTCATTCAAAGATACAATAGCTAGCAGTCAAAGCAAATACTAGG
TACTTTCTTAGTTAAATAAACTTTTTCTCTTTTGAGAATTTTAAAAATTTCTTTTCAGGTTGGATAAAAG
AAAATCCAAGTCATTCCCCACCTTCCCAAGAATTAGTGTGGTGATTGTCAAGTAGAATTAGGACCTGAGG
AAAAGAAAATAGGCAGAAAAACGACTGCTTCCTTTATGAATTCAACCCAGAGCAAAGAGATTTGTCATGT
TATTGCAATCTTTTAGGCTGCTAGAGAAAGCAAGTCTTTCTTAATACACTACTGTAAAATCAAATGGGTG
ACAAAGTTTATAGCAAACATTTAATATATTCCTTACTATTTGCCAGGCACTGTTCTAAACTCTTTCACAT
AAAACTCTTACATCCTCATAACAATCCTATGAAATAGGTACTATTGTTTCCACCATTTTACATATAAGGT
AACTGGGACACCAAGAGGTTAAGTAGCTTACCAAGTTTACATAGCTACCCAAATGGTAAGTGGTCCTGTC
TTACTTACCACGTTACCAAAAATGCCAATGCATGAAAAAAATGATCATTAAAAAACATCTAGCTTCTTAG
GTGTGTATTAGATATTTTCCAGGGATCTGACTAGCAAATTTTATGTATCCTAATCATCACAAACATCATC
TACCTCCTCTCATTTCCCTTTTACTCAAAATTAATATTTAGGTGGGTCCTAAATGTGTGTGGTATGGCTT
CTAATTTATTTATTAATACATACTTTTCATTTGGTCTAAATTAGAGATAGAATGCTGGGCATTTATGAAT
AAATGTGCATGGGACCAGTGCAAATTTTTCATGTCATTTCAAGCGACACTTATTTTGTCTTTGAAAAATA
ATTTTATATCTTTTATCAAATTAAAATTATTAAGCTACATAAAAATGTTCTCAACAGAGGATTTCAGACT
TACCTGAGTTATCTTAATAATTAAATAACTATTGGGTATTTATAAACGCAGTCTAAGGTTGGTATTTTTC
ATCTTTTAGGCTATAGCTGTTTGTATGGAAAATGGCAACTTTAAAGAAGCAGAAGAAGTCTTTGAAAGAA
TATTTGGTGATCCAAATTCTCATATGGTAATTATTTAAATTAAAACCATAGAATTTTAGAAGTGTTTATG
TAGAGTGAGGAAAACTGAAAGAAAATTGGAGGAGAAAAGAAATGCTTTATCTTTTAAACAGTATAGAATA
ATACCGTGCAACAGAAAAACTACGCAAGTCACATATGTGATTTAAAATTTTCTAATAAGCTGGACGCAGT
AGCTTACACCTATAATTCCAGCACTTTGGGAGGCTGAGGTGGGAGGATCTCTTGAGCTCAGGCATTCGAG
ACCAGCCTGGGCAACATGGAGAGATCCTCATCTCTCCAAAAAGAAGGAATAATAAATAATGAAAAATAAA
TACAATTTTCCAGTAGTCATATTAAAAAAGTAAAAAGAGGCCCGGTGCAATAGCTCATGCCTATAATACC
AGTACTTTAGGAGGCTGAAGCAGGAGGATCATTTGAGGTCAGTAGTTCAAGAGCAGCCTGGGCAACACAG
CGAGACCCTGTTCTCTACAAAAAAATTAAAAATTAGCCAGGTGTGGTGGCGCAATACTGTAGTCCTGAGG
TGGGAAGATTGCTTGAGCCTAGGAAGTCAAGGCTGCAGTGAGCTATAACGCTGCCACTGCGCTCCAGCCT
AAGCAACAGAGAGAGACGCTGTCTCTAAAAATAAATAAATAATAAAATAGTATAGTATTTGCATATAACA
TATGTACATCCTCCTGTATAGTTTAAATCATCTCTAGATTATGTATAATAACTTACACAATGTAAATAGT
TGTACTGTTTAGGAACAAAAAGATACAAAAAATCTGTATGTGTTCAGTACAGGTGTAATTTTTTCCCCCA
TACATTTTGTGGAACAAATATATGCATTTCTTTTGGGTTTATATGAAGAGTAGAACTGATTAGTCATAAG
ATGTGTACATACTCAGTTTTAGTAGATATTGCCAAACTGTTCTCCAAAGTAGTTATGTCAGTTCCTCCAG
TAGTGTGAAAGAATTCTAGTTGCTCCACACACCTTGTATCTGGGTTTTCATTTTAGCCATTCAGGCAAAT
ATGTAGTGCTATCCTGTTGGATTTTAATTTGTTTCCCTGATGAATAATCAGATTGAGCACCTTCCCTTGA
TAATGTTAGTAGATAGAGTTGGAGAGTGACTACCTTAATCCAGTAAGGAACCTGGGCCTAGTAGAGAGTA
GGTTGGGCAGTTTTGGCAAGGCTTTGTCTATCTCTAATTTAACCCTGTACCTGGGGTATAACCCTCCAGA
AATTTCAGCCAATAGCCTGATATGTCTGTTGGGTTTTCTACCCTTAATATTTTCTCCATAGCACTATGAG
ACTACTGAAAATTCTACTCTGCTTATACCTCCCAGAAGAGGAAAACTTAATGGCCAGGAAATATTAAGAG
GTTAAGATAAGTACTTGACCTCACTAGTATTAATGTTGCCTCATTAGGGCAATATTAAGTACTATATTGA
AATACCACTTTCCATGCCTCAGACTGGCATCAATTAAAATGTCTTAGAATATCAACAGTTGTCTCAACAA
ATTGAGAAATGATTGTACTGCCTGCCATAACTTCCTTTCAAGACTGTAAATTGGTACTTCTTTGGGAGAT
CATTTTAGCAAAATATTTTAAGCATGCATTCCAGCAATTCCACTTCTAGACAATACCTTAGAATAGCTGG
TTTCACTTTTAACTGCATATTTTAAAAGCCATGAAGTTTAAAGGCAACAAAAAACCAAGGCCTTCCTGAG
ACAGTTGCTCTGGGATTGAGCCTAAGAATTTTTTTAAAGTTCTACTTTTTTTATTGTGCAGCCAGCATTG
AAAAAGCTACTGTCCAAGAAAAACACATACAATGCACAGAAGATAATGTAGAAAAATGTTGAAGAGCGAT
ACAATAATTGCAAGTAGCCTAAATCCCTCAATAGGCATGAAGAATGGATGAATAAGTGATGTTTTTGATC
AGTGTAGTACTTTACAATGGCAAAAATAACTACTTCTAGGAATATCAAATCAGCATGGATAAATGTTGTA
AACATAGAGTTGAGTGAAATATAGCAAGTTTCAAAAGAATACGCATCGTACAACACTATTTGTAGAGTTA
AAACCATGCTAAGCAATGCTACATATTTGTGGATACATACATATGTAGTGCAAGTAAAGGAATTCCTGGG
AATGGTGAATTCTAAATTCAGGGTAATGGTTACAGTTAGGAGAGGAAAGGGAATACAATCAGATTATGGT
AGTAAACAGGGATTTTAACCATATTGATTATGTTTTATTTCTGAAACTGGGTAATGGATACTCTGGTTTA
TAATACATATCTTTTTGAATCTGTCTAAAATGTAAAATATTTTTTAAAATGAGGAGAGGCCAGAAGAAGT
CAGAAAAAGGTACATTGGCATGCCATTTAAAACTTAATTTTAAAAATATCTGTGTCTCTTGAGTTTGTAA
AGTGATTTCTTTTCAATTAAATAATATGTGACAAAAATCGTTGTATCTGTAACTTTGTGTGATTTATGTT
AATATATTTCTTCTGTTTTCTTTAGCCTTTCAAAAGCAAATTGCTTATGATAATCTCTCAGAAAGATACA
TTTCATTCCTTTTTTCAACACTTCAGCTACAACCACATGATGGAGAAAATTAAGAGTTATGTGAATTATG
TGCTAAGTGAAAAATCATCAACCTTTCTAATGAAGGTATACATATTATTCAAGAGTGACTAATAATAGAC
TTAAAGGAAACGTTATAATAAAGGAGAATACTTGAAATAGAAATCCTTTAAAATTAATCAGAATTTTTCA
TTGTGGTTTTGTAGAAAGGTGTGGTAAAATGTTACATTTTGTGGACCTAATTAAAAGATTTATGAGTGAT
ATGTACTTTTGTAACTTTTCAAAATTAGTTTGGGGAAATTTTCAAAAGAAAAAGTTGAAGGGAAAACTTA
GAATGAATTAAAAATAAGAAATAGGCAAAAGCAAATATTTTAATTTAAGTAAATTTTAAAGACTTTTTAA
ACATATTTTTGGGGAAAAATAGAATTGTATTAAAGAAGGGCCTTTTAAAATTTTTCTAGGCCAGGCGCGG
TGGCTCATGCCTGTAATGCTAGCACTTTGGGAGGCCAAGTTTCAGGAGTTCGAGACTAGCCTGGGCGACA
TGGTGAAACCCCTTATCTACAAAAAAATACAAAAAAATTAGCTGGGCCGGGCGCGGTGGCTCACGCCTGT
AATCCCAGCACTTTGGGAGGCCAAGGCAGGTGAATCACACGGTCAGGCGGATCACACGGTCAGGTGGCTA
ACATGGTGAAACCCCGTCTCTCCTAAGAAAATACAAAAAAATTAGCTGGGTGTGGTGGCAGGTGCCTGTA
GTCCCAGCTACTCAGGAGGCTGAAGCAGGAGAATGGCATGAACCTGGGAGGCAGAGCTTGCAGTGAGCTG
AGATTGCGCCACTGCACTCCAGCCTGGGCGACAGTGAGACTCTGTCTCAAAAAAAAAAAAAAAAAAAAAA
AAAAGCTGGGCATGGTGGCATACACCTGTAGTCCCAGCTACTTTGGGGAGTTGAGGCTGTAGTGAGCTGA
GGTTGAGCCACTGCACTCCAGCCTGGGTGACAAAATGAGACCCTGTCTCAAAAAAAAGACATTTCTGGTC
AGGCGCAGTGGCTCATGCCTGTAATCCCAGCACTTTGGGAGGCCGAAATGGGTAGATCGCTTGATCCCAG
GAGTTTGAGACCAGCCTGGGCAACATGGCAAAACCCCATCTCTACCGAAAAAATAGAAAAATTTAGCCAG
ACATAGTGGCACACGCCTGTAGTCCCAGCTACTTGTGGCGGCTGAGGCAGAAGGATTGCTTGAACCCAGG
AGGTTGAGGCTGTAGTGAGCTGAGATTGAACCACTGCACTCCAGCCTGGGTGACAAAGTGAGACTTGTCT
CAAAAAAAAACACATTTCTAGTCAGGCGCGGTAACTCACGCCTGTAATCCCAGCACTTTGGGAAGCCAAA
ATGGGTGGATCGCTTGAGCCCAGGAGTTTGAGACCAGCCTGGGCAACATGGCAAAACCCCGTCTCTACAA
AAAAATTACAAAAATTTAGCCAGGCATAGTGGCGCATGCCTGTAGTCCCAGCTACCTAGGAGGCTGATGT
GGGAAATCACCTGAGCCCAGGAGGTTGAGGCTGCAGTGAGCTGTGATTGTGCCACTGCACTCCAGCCTGG
GTGACAGAGTGAGACCCTGTCTCAAAACAAACAACAATAAAATTTTTCTAATAACATTTTATTTTCTGTC
TTGGCCTCCAAATTTTTAAGTTGACAGTGTTATAGTGGAGCATCTTGATTGATTTGAGATAGTTTTTATT
TGAAAATATCTAGAATTTTTATTTTTTTTTTTTTTCATGCAGCCTTTTCTCTTAAAATTGCCTAGAGAGC
ATTTTGTCTATCAAATATATTTACAAGGAAAGACCACCTTCGTAACGCTGATAAACCAAGCAGATAGCTA
ATGTAGCTGCTGGCAGTGTCATAATTATTGGTGAAAATAACAACACAGTGCCCTTCATTGGCCTGTGAAC
AGATACATTAGCTATGACTATCAAAGAATTACAAGGCTTAATTTAATGCTATTTGTTTAAAATGGCTTAA
TGCATTTCTTCCTATCCTTCTACCTCCACGCACGTTTTTTAAGGCAGCGGCAAAAGTAGTAGAAAGCAAA
AGGACAAGAACAATAACTTCTCAAGATAAACCTAGTGGTAATGATGTTGAAATGGAAACTGAAGCTAATT
TGGATACAAGAAAAAGGTTTGTAATTTAATCAATTTGTATATTTTTTGTTTTATGAATGTTCTGTCTTTA
TGTAAAATTGATATGTCTTTTTATTAAAAATAATTATTGAGTAGCATGTTATCTTGCTGATGATTAACAT
TTAGGTATTTGAAGTACTGTAGAGTTATTTCATGACTTGTGCAAATTATATATGCTAAGCAAAAGTAGAG
AATAAGAAAATATTACAGTTTAAATAATGTGGATAGTTTACCATTCTTGGGGTATGCTTAGGAACAAGGG
TGAGGTTGGACTCCTGTGCCCCTTAGTTGTGTTTAGATTCTACATGTTCTCATTATGTGATTCATGTTTC
ACTGTACTAAGAGTCTAGTATTTTAAGATATTATTCATATATCTTCTCTAAGCATAAGCTCACTGCTTCA
TCTGGTGCTCAACTGAAGAAATAGTGATTTGCTTTAATACTGGAGGAAAACAAGTTGTGTCAGTCCTATA
TATTGAGAGATAGTTGTTAAGTCTCCAAAATTATTTTTTCTTTAGGGATTTTCAGGGGTGATTGTTGTCT
TTTGTTTTAGAAGCTTAAGTATCTTCCCCTATTGAGAAAGATATAACTCTTAACATTATAAAGATCTTCT
TTTCTGTTGAACCCATGGTCTGTTTTCTCTGTTCTTCTACCCTTAAACCAAGGCCAGATAATCAAATCTC
TGTGGTCTCTGGATGGGTATATTTGGAAATGGATGTAATATTACTTTCTTCTTGGGACCATGACCTAAGT
GTTGCAGGGACCCCATCTTAACTACTTATTTAGAGGTGGAGATTGGGGTTCAAGTTGGATCTACACTGCA
TTTGTCACATCTTAGGACTGAGGTTCTGTCATCTTATAGTGGCCCCTCCTATTGTTCCTGCCTGTCAAAG
CATGACTTGTTCCTCAGGGGTTCATCTCTCCAAAGGCTGTACTAAAGTCTTATTTCACATTGATGTACTT
TTTTCTAAACTCCAATTTAAGAAGTAGAGATTCCATTTTTATTGGCCCAATTACTTCCTTTTGTTTCAAA
AATCAAAGTTGAATGATACTAATACCCAGTACATTCTTGTGGCCAAGCCATCCTGCTCATGATAATATTG
CTAATACCATGGTTCTTACAGATAATCATGAGCACACTTCCTATGGACTCTCCTTATATAGTGCTACCCA
CTTATTTCAGATGCTCTCCTAGACACAAACACAGTGCAATCCATGTGTACCAAACAGAACTCTTCATGTT
CTCATTTGCCTCTCTACACCCTGCCTCTCTTTAACCTCACACATCCAGCTGCTCTCCAAGTATTAGGAAC
CGCACCTCAGAAATGCTTCTCCAATCTATTTTTATCTATATAGCTTTGTACCATTCAAATCCATCCTTGA
CATAACTTCTAGAGTTAGCTTCCACAAAAATAAATCTCTTTAGATCACTCCCAGCTTAAAAACCTTTTGG
CTCCTATTTCCTACTAGATGAGTCAGAATTTATTGGCATGGCTTATAAAGCCTTACGTAGACCCATTTTT
TTTTTTTTTTTTTTTTTTTACATACAGTCTTATCCCTAAGCATGAACTCCTCCCTGTCCACATACATGGT
ACTTTCTAGCAGCACAGAAGCTTTGTTCCTTCTATTTGAATTCCTATCATTTTATTCTCTCCAAGGCAAG
CTCTTCCTTGCCATTCAGTCTATCTCAGAGATCACCTATATGAAGTCTTGCCCAGCTCTGTAATCAGAGT
TGTTACTTCTGTGCTTTCATAGCATACATCTCTATAAAAGGACTTATCTTCGTTGGTAACTTATTTGTTT
ACCTGATTCTTTCAATAGATTGAGAATTATTTGAAGGTTCTTACTTAGCTTTGAAGCATCTTAATTAGCT
TGGTATCCTTGTTGCCTCATTCATTACCAGGCATACATTAGAACTTGCTGTACACTAGTTACAGGGAACA
AAACTAAAACCAGAAACAGGGTAAAGATCAAGAATCGAGCAAATACCAAAACCATCGTGGAAGCAGAACC
AGGGCAAATAAAGCATCAAACCACCAGTGTCATGACAGTTACCAGTAAGGATTCAAGTAAAGCAAGTATA
GAATCAGAAGTGTGATTGTGAGGTCAGACAAATCCTAGAAAAATGGAAACCTCCATGTTATGAAGACTAG
ATGAAATAAATATTTTGCTCTGATAAAGCTTATACAGTTGTTCCTTGGTGTTTGAAGGGGATTAGTCCTA
GGACATCCATCCTTCATGAATACCAAAATCCATGGATGCTCAAGTGTCTTACAAAAATGGTGTAATAGCC
AGGAACAGTGGCTCACACCTGTAATCCCAGCACTTTGAAAGGCTGAGGCAGGAGGATCACTTGAGCCCAG
GAGTTTGAGACCAGCCTAGGCAACATGGCAGAACCTTGTCTTTACAAAAATTAACCAGTCTTGGTGGTGC
ATGCCTGTAGTGCCAACTACTTAAGAGGCTTAGGTGAGAGGATTGTTTGAGCCCGGCAGGTCTGCAGTAA
GCTGTGTTTGTGCCACTGCACTCCGGTGACCCTCGGCGAGACCCTGTCAAAAAAAAAAAGGGGGTGTAAT
ATTTGTATACAACCTACACACATCCTCCCATATACTTTAAATCATCTCTAGATTACTTAAAATATCTAAC
ACAGTGTTGAAATAGTTGAGATGGACAGCTTCATGGAGGTAGTAGTCTTGAACAGGGGCTTTTGTAGGAT
GGTTAAGATTTTTACAGATGGATAGAAGAGGAAATATCATTTTAGGAGAGAGGAATATTACTAGTGAAAG
CATGGAGGTGTAAATAGTCATGCTGTGTGTATGTGGAAACCATTTTAACTATAACAAACGGGAACTTAAT
CGATTCCCAAAAGTTCAATTGGAGACTGGTGGGGAGAACCAGAAATTAAAATTGAACCCATCAAATTGTA
TACTTTAAACATGTGCCATGCTTTTTATGTATCAATTATACCTTAATAAAGCAGTTTTTAAAACTTGAAT
TCATGGAAATAAAGTGCCGTAGAAGAGTTTTTGAAAGAGTGTTATAAATGCTTGACTACACCATTAATTA
TGCTTATGTTTGGAGAAACTTGGTTTAATACCTCTTATAAGCATGTCTGGACAAGAATTAGCCATGTTGT
AATGGACTGTATATGTCCCGATGTTACCAAGGTTTCTTTCCAAAGTTATTTTTTTATAAATTGAAATATG
CCTTGACTATGTGGAAAGTACTATCCTTATGAAAAGTTTCTTTTGTTTACATTCTTACAAATTTTTTCTT
CTTTTAAAGTGTTAGTGACAAACAGTCTGCGGTAACTGAATCCTCAGAGGGTACAGTATCCTTATTGAGG
TGAAGTAAATTGACGTTTTTCTTCTTTGTCTTTCAAATCTTTGATTGAAGCAATTCATGCAGACCTAATA
AAAACTACCACATAAATGGTACAATTGAAAGAATATCTCAGGGTCCTAGTTAGCCCAACAGATAGTCACG
ACTCTGAGACATCTTGAAATTCCAAACTGTAGAGACCTGCTCTGAGACTGGGTTGTTTCACATTGTTTCT
ACAAAGCCATTATTGGAATACACATATAGGGAGTTCTTGAGCTTAAAACAGATGTTTAGTAAACACTTCT
CTGTCACAGAAACAAAGATATGATACTGGGGCAGGAAGGGGATGGCAATTACATTTCTAATGAATGCCCC
ATGCCAATTTTATGAAAGTTACTTGAAAAACTTTATTTGATCTAATAGAATCGCTGTGACAGACAATACA
ACCTGTCCCGAGGGCAGAAAGTCTGCATGAATGATTTCTAACATAGAGCAGTTGGGATCACAAGTGGAAC
CATGGGGAGGAGGTGGCTGACTTCATGTGGAGGAGGTGATTGGACATGAGGAACTGAGAGGCCAAGATGG
TATATGAATTGTCTGTGAGAACACTGAAATAATTCATAATAGCAGCACTTAGGTGTAGAGAAAGATCTGT
GAATGATGAAGAAGTGCTTGGAAGGATGGTAAATGGCTGTGAAGAATGATTGAAGGTGATACATCTCAGT
GGCATGAATCCTCAAGGCTGAAGTTTCTATGGAAGCAGGATCTTGAAAGTGGCATTCATTAGGAAATGAA
AACTGCTGTCTCTGGAGTCCTATAGTTTATGGAGGGTGGAAGATTAAGCCAATCTCTACTTGAGAAGGCT
ACCAAGGAGAGAAACATTGTCCCCAGGGGAAAGCTAGATTTTAGACAAGGCAAAGAGCGGAGAGGAATGG
GTGAAGAGATTGAGAATGACATTCTAGAAAGGACAATAGAAAGTCTTAGGAAGGAGGGACAGAGGGGAAG
AGAGAAAGGATGGCTGGTCACCTAGAGTATCTATAGGGATAAAAGTATAGCTAAAATAACCTACATTTTA
GGTGGAGATAATACAGCTGCCTAAGCTTAGGTGAACTGCTTTTTGAATCTTAGTTTGCTCCTTCATAAAA
TTGGATTAATATAATACCAATTTGGCAGAGTTTTCAAAAAAAATGATGCCACATCCTGTGTTTCCAAACA
GCCTTACCTTTCAAAACTCTTTTTGGAAGCTGTAAAGATAATCAAGTGATACCTGCCATTATGTAAAGGA
ATTTTGGAATCCGTCTTTGGGGAAATAAAAGCCATAAGAGAGAATGAAAGCTAATACTTTGTAGTTTAAG
GATTTTTTTCCTTGATGTATCTAATTGGAAGGCCGAAAGAGTCTTGTCTTTTCTTTTTTTTGCCTGCTTC
TTCTGAGACATCAAAAAACATCTTGATTTCTGCGGAGTAGATGAGATGGAGAAAGATGAGACAGGCAACA
GAAATCTTAAGAGGCCACAGGTTAAGATGGGGTACTAAGCAGGGAGAGCACCAAAGGAAGTAGGCCAAAG
TATTAATAGAAGATCCTAGAACAGATTAAGGCAAATCATTTACCTGTTTTCCATTGCCTTACTATCTTTC
TGGAGCCAATTACAAACTTTCCTGTTTTATTTTAGGTCTCACAAGAATCTTTTCTTATCTAAGTTGCAAC
ATGGAACCCAGCAACAAGACCTTAATAAGAAAGAAAGAAGAGTAGGAACTCCTCAAAGTGAGTACTGTTA
TAACAGTTTTAGAAGGGGAGAATTGATGTGTCCTCAAACAATCCAGCCATTGACTTTACCACTATAGCAA
AAAAACACACACTTCAGAAAACTGAAGCACAAAGTCTTGACTTCATAGAGTTAATTGAGATAGTGTTTAA
TTTAGAAAGTTAGCTATTTTGACTGTCTAGCTTAGGGTTCTTTTGCTATGGTGGTACACTGCTTTTCTAC
CTTCACTGTGTTCAGCAACTTCAAGTATAGTCATATGCCACATCATGACATTTCAGTCAAAGACAGACTG
CATTGCATATAGGATGGTGGTCCTGTAATAATGTAATGGAGCACATATGGAAACTTGATAATGTGGCACT
TGATATTGGCATTGCATATCAAGTAGGGGAAATGATTGATAATTAGCAATGGTACTGGGAACCGTGGGTT
TTCTATATGAATATATATATATATATGTATGTTTGTGTAAGTGTACTCTTAATAATGTTCACATAAGGAC
AAAATCACCCAACGCCCCATTTCTCAGAACATATCCCCCTTGTAAAGCAATCCGTGACTGTAGTTACTTT
TCAGAAACATTAAGTACAGTTGATTCTTGTTATTCACAGTAGTTACGTTTAATAAAGTCTGAGAGCGTTG
ACTTAGCAAATTACAAGTTTCTTTTTGGTATGTAGTCATTTAACAAATGTTTATTGACCCCACAGTAATA
TTTTAGGTACTGAACTGATACTGAGATTTTTTTTTTTTATACTTTAAGTTTTAGGGTACATGTTCACAAC
GTGCAGGTTTGTTACATATGTATACATGTGCCATGTTGGTGTGCTGCACCCATTAACTCGTCATTTAGCA
TTAGGTATATCTCCTAATGCTATCCCTCCCCCCTCCCCCCTGAGATTTTTAATATGGATCTAAACCTAAA
TACTTATTGATAAAAATAATAGACATAATGAAATTAGGTTTATTTCTCCATGCTCATAAGGTACCATCTT
GGTGCCAGGCAAATGATAGGCCTGCAGTAAATATTATAGAATAAATTTGTTCATACCTGATGGCTGATGA
CATGGCAACCATTAGCAAATATTTGTAAATAGAGTTCTAAAATATTTACATTCATAGATATCATTCCCAT
TTTTAAAGATGAGAAAACGGAAACTCAGTAAAGTTATGTGCTTAATGTTGGTGGAGTTCCAAGATTGGTT
TTCTATACCTATTGCTGCAAGACGGCTGGGAATCATTTTAAATGAGAATAATGTGAGCATTTTCCTAGGG
TTTTAATTTCATAGTATGAAATTCAAAGAAAAGGAAATGTAGGCCAGGCGCAGTGGCTCACGCCTATAGT
CCTAGCACTTTGGGACGCTGAGGCAGGTGGATTGCCTGAAGAGCTCAGGAGTTTGAGACCAGCCTTGGCA
ACATGGCGAAACCCCGTTTCTACTAAAAATAGAAAAAATTAGCCAGGCATGGTGGTGCCTGCCTGTAATG
CCAGCTACTCAGGAGGCTGAGGCACGAGAATCCAGCTTGAACCTAGGAGGTGGAGGTTGCACTCCAGCCT
GTGTGACAGAGCGAGACTGTCTCAAAAAAGAGAAAACATGGTTTATCAGCAAACTCGAAGACTATAAACT
CTTTGCAAACTGGCTTGACAGTGACTGTGGCTATTGAGTCTATAAGGTCTTTGAGTTTTAGCACATTTTA
TTAGGTGTCAGTAATAAGTACGTTTTCTTAAAAAAATTCAGTGTACTTCAGTGGTACCCAGGTACCCCCT
CTTTATAGTTTGAGGCTAATAATTTTGTTTGTGCTAAACCTTTTTTTCTCTTCTTTTTCTGTTTTTGGAG
ACAGAGTCTTACTTTGTTGCCCAGGCTGGAATGCAGTGCAGTGGTGAGATCTTGGCTCACTGTAGCATCT
ACCTTCCCAGGCTCAGTTGATCCTCCCACCTCAGCCTTCCAAGTAGCTGGGACCACAGGCACATGCCGCC
ACACCCTGCTATTTTTTTGTTTGTTTGTTTGTTTGTTTGTTTTGAGATGGGGTCTTGCACTGTCACCCTG
GCTGGAGTGTGGTGGATCAATCTCGGCTCACTGCAACCTCCATTTCCAGGTTTCAAGCAATTCTCCTGCC
TCAGCCTCCTGAGTAGCTGGGATTACAGGAGTGTGCCACCACACCCGGCAGATTTTTGTATTTTTAGTAG
AGACGGGGTTTCGCCATGTTGGCCAGGCTGGTCTGGAACTCCTAACCTCAGGCAATCCGCCCACCTCGGC
CTCCCCAAAGTGCTGGGATTACAGGCGTGAGCCACCACACCCGGCCTTTTTGTGTTTTTTGTAGAGGTGG
GGTTTGGCATGTTGCCCCTGCAGGTCTCTAACTCCCAAACTCAAGCAATCTGCTTGCTGTAATTGTGCTG
AGATTACAGGCATGAGCCACTGCTCCCAGCCCTAACCTTTTTTTCAAAAACTAATTTTTGTGTTGTAAAA
TTGACATATGCTTAGGGAAAAAAAATCAAGTTAATTCAGAAAACTTGAAAGAAAAAGCCAAATCATCCAG
ATTTCATCAGTGCAGAAAAACTCACCAGCATTGAGTGAAATTATTAATATTTGAGTTGAAAGCTTTTTAT
AGGTAAATGCATTCAATCCTCAAAACAACCTTATGAGATTAGTGCTTTTTTTTTTTATCATCTCCATTAT
ACAGATGAGGAAGCTGAGACTTAAAAAGGTAAATAAAATGCCTTTGGTTACACAGTGTATGAATAGAACA
GGAGCCAAAATTTGAAATCAGACAGTCTGACTCCAGAGCCCAGGCTTTTAATTGCTTAGATGGACTACCT
CAGTAGAAGAATGTATAGATTGAAAGCAAATCAAATTTTAGAAAAATGTCATCAAGTTACATATGTTACT
TTAAAATATATTTACATTTAATTTTTAATATGTAAAACTGAAGGATTCAGTACTTCAGATAAAGGTGGTT
TCACAAGAAATCTATCAGTAACCTTTATGATCTGTGTAAAATAAAGAATATGAAATGCTTTAAGAGGTCA
TTCAACTTTAGTTAATCAACTTTAGTCTGTTGATTGACCTTAGCAAAAATTTCTTCCACCAATCCCCAAA
TTTTCTTGGTTATATTTTATATAATCAATAGTTGTAGCATTGTTTTCCCATGTTGGATTTTCAGTTCAAA
ATACTTTTTCATTTTTGTAGAGACTTTTTTACTGACCTATGAGGGAAGTTGTTTAATTTCCAAATATCTT
TCTGTTACTGATTTTTAGTTTAATTCTATTTCAGTCTGTGAATATGCTTCGTAGATTTTTTTTTTTTTAA
GATTTGTTTTATGGCCCACAGTATGGTTTATTTTGGTCCATTTGGATGTGCACAGAGAAGAACGTGAATT
CTGGCATTGTTGAATGAAGTGTTTTAAAATGTCAATTAGGTCAAGTTTTTAATAATGCTGTTCAGGTATT
CTATATATGTACTCATTTCCTGTCTATTGTTGTATCAATTAGAAAAGTGATAAATCTCCCAAATATAATT
ATGAATTTGTCTTTACATTTATGTGATTATTTGCTTTTTGCTATTTTAGTCCCCTAGCACCTGTTTAAAC
ATTTCTCCTAATGCTAAAATCTCTCCATCAGAGCTAGTGTGGTTTCCATTTTCCTGACTGGAACACACCC
AGTATATATGTCAAAATCCCATATATGTCAACATCCCATGTTTGTAGTAGGGTAATCTTATTGTTTTTCA
AGAGTATCTTGACTATTCTGGTGTTTTGGTGTTCCATATTGTCAGGGAAAAAACCTAGCCATAGGGCTTT
ATAAGCAGTAAACCTGCTGCTACTTTATAGATACAAAAGGTAGACAACAAGACTTAGTGACTTAATGAGT
TAGAGTAAGACAGATTATTATAGCAAACAACATGGCATTGATGTAGTTGTTCAGATTTCCCTACCCCAAG
TCCGGTGGACTATGCAACAGGCCTAAATGGATGCTGCGAAGGCAGTAAGTTATATTTCAGCTGAGGAAAA
CTCAGAGCCAAGGGCCCAGTGCTTTTTGAGTAAGCAACAAAACATTGTAACAAATAGCAAACAAGCCAGT
ACCCTCTCCCAGGAGAGAGCAGTTGGCCTGGTCATCATGTTGTGGTTTCCTTGACCTGCTTAATTGCCTA
TCTGACTGGCTATAGAAAATGCACTATTTCAGGAGATGAATAAGGCTTTTCTACATTCAACAGGAATTGC
AGGGGTGCTCAGAGCCAATAGTGGACTACCTGTCTTAACACATAATTTTAGAATAATTTTGTTAAACTAT
ACGAAGAACCCTGTTGAGATTTTTATGTGAAATTGCATTGTTAACAATAGATGATTTTGCAGAGAACTGA
CACCATTACATTAAGTTTCCTGTGCATGAACAAAATACATTTCTCTAACATGTTTTTATATATACAGTCA
TCCCTTGGTTTTCACAGGGTATTGGTTCCAGGACCCATCCCCCCGCCCCCACAGCAGGGATACCAGAATT
CTCAAATGCTCAAGTCCCTTACATAAAATGGCATAGTCTTTGCATATAGACCTGTGCACCCGCCTACCAC
ATGCTTTAACTCATCCCCAAATTACTTACAGTAACTAAAACAATGTAAATGATAAATAACTAAATTGTAG
TAAATAATTGTTAGACTATATTTTTTATTTGTATCACTTTTATTATTGTAGTTATATATAAATATATATA
ATATATATTTATATATAATATATTATATATATATGACCTATGAGGGAAGTTGTTTAATTTCCAAATATCT
TTCTGTTACTGATTTTTAGTTTAATTCTATTTCAGTCTGTGAATATGCTTCATAGATTTTTTTTTAAAGA
TTTGTTTAATGGCCCACAGTATGGTTTATATGTAATATAATATATAATATATATAATATATAATGTACAT
AATATATAATTATAATATATATAATAATTATACTACATATAATATATATCATATATTATATAAAACCCCA
TATATACTATATAATTAATAATTTATATATTATAATATATAATAATTTATATTATAATATATAATATAAT
AATATATATTATATAATATAATTTATATATAATTATAATATAATAATTTATATATAATTATAAAGCTTTA
CTATGTATATAAAACATGTTATGAGAAGCTTTATCTATCTTATCTACCTATCTACTTACCTACCTACCTA
CCTAACTGTTGGCCCTCTATATTCACAGATTCCACATCTGTGGATTCAACCAACCTTGGAATAAAATATT
TGGGGGGAAAATACATATAAATTTTTATATATAATATATAATATATATTATATATGTTATATTAACATAT
AAAATATATTAAATATATTTATATATAATAAATTTATATATTTGGAATAAAATATATAAACCTTGGAACC
TTGGAATAAAATATTCGGGGGGGAAAATATATATATTTTTTATATATAATATATATAACATATCATAAAT
TTATTATATATATTATATATTATATATAACATATATAATATATATTATATGTATAATAATATTATATTAT
ATATAATATATATTATATAGTATAATATTATATTATATATAATATATATTATATAGTATGAAATATATAT
TATATATAATATTATATAGTATAATATATATATTATATATAATATATAGTATAATAATATATATATTATA
TATAATATATAGTATAATAATATATATAATATATAATATATAAATATGATATATAATATATATAAATATG
ATATATAATATATATAAATATGATATAATATATAATATATATAAATATTATATATATATAAAACATATAT
ATATTTTTCCCCCCGAATATTTTATTCCAAGGTTGGTTGAATCCACAGATGTGGAACCTGCGAATATAGA
AGGCCAGCAGTAGGTAGGTAGGTAGGTAAGTAGATAGGTAGATAGATAGATAAAGCTTCTCATAACATGT
TTTATATACATAGTAAAGCTTTATAATTTACTACATACAAATGTATCACTTTTGATAAAGTTATCTAAGA
GGCCATGACCTGATGGCTCATACCTGTAATTCCAGCACTTTGGGAGGCCAAGGCAGGAGGATTACTTGAG
CCCAGGAATTAAGAGACCAGCCTGTGCAATATAGTGAAACCATGTCTTTACAAAAAAATTTTAAAAAATT
AGTCGGGCATGGCGGCCGGTGCCTCTAGTACCAGCTACTCAGGAGGCTGAGGTGAGGGAGGATAAGGCAG
AAGTATCACTTGAGCCCAGGAGTTTGAGCTGTGATCATACCACTGCACTTCAGCCTAGGCAAAAGAGAGA
GACCCTATCTCTTAAAAAAAAAAAGTTACCTCTAAGAATGTTAGATTTTTTTGTGGCTATTGTAAATAGT
ATCTTTATATTGTTTTAATTAAATTATTTTAACTGTTTGTTGCTAATGAATAGAAATCCTATTGCTAATG
AATGAAATCCTTTTTGTGTATTGATCTTACTGATCTTTTAATTCTATTTGTAGATTCTCTTAGGTTTTCT
GCCTAAACAATTATCATTTTTCAGTAATTACAGTTCTGTTTCTTTTCAGTTACATATATTTGTCTTACCA
AACTGACTATGAATAAGAAAGAAGGAGTGAGGAAAGGATTATGCTTCTTAGTACTCCTAAAACATTATCG
TAAGAAAATTTCAACGATCCTATGCAATAAATATTTTTCCTTTTTTTTTAAACAGATAATGGAAAGACAG
GTTCAGGGATGTGAAATGATTTATTTAAAGACATATAAAAGAATCAAGACTCAAAACACTCCCCCTCTGA
TTTCAAAGGCTGATCTTTGCACTATACCATACTGCTTCCAGATTTGAAGGAAAAAGTTAAACATGTACTT
TTTCTTAGAATAGCTTAGAAAAGGAATTTCATTATAATCATTAAAAATATAATTGCTCTTTTTTCTTTAA
TGTAAATCAATATTTATGACATTATTTTTCTACTTTTAGGTACAAAAAAGAAAAAAGAAAGCAGAAGAGC
CACTGAAAGCAGAATACCTGTTTCAAAGAGTCAGCCGGTAACTCCTGAAAAACATCGAGCTAGAAAAAGA
CAGGTATTTGGTTTTTTAAAATTCGAATAACGCTTATGGACATTAAAGTTATAACTTGAATAATTGTGAT
TATTTCTGTTCAACCTCCCCAAAATTTTGAGTGTAAAATATTTGCCCTTTGCCATCTTTTATTTTGCCGT
CTTAGATAGGTGACTAATCAGCCTATTCAAAATGAAATACTTCACCCATATTTAAAAAGTCTTAAGTTGG
GTGTTGGTACTGACATCTGTTTGGAATTATTGTGATACTATCATAAAATCTGTTTATTAGATGTAAGGTG
GGACTTAACTCCACATTATAAGCTTTGATGTTTAAATTTTCCTAACAGGGATCCAGTGTAAAAAGAGTTT
TCTTTACTTAAAAACAAAGAAAAGATACCATTTGATCACAAAATTTTATTAAGGTGGGTCAATTTAGGGC
TTGATTCATTTGCTTCCCCTTTCAGTGACTATAGTCTTGTGCCATCTGTCCAGTGTCTGAAACCATTATT
TTATGTATTTTTTGATTGTCTAGTTGTTCACAGCAGATGGATAAATCTGGTCTTTGTTTTTGTGGTGTGT
GTGTGTGTGTGTGTGTGTGTGTGTGTGTGTGTGTGTGTGTTTCCCCCTTTTTTTTGTAGAGATGGTGTCT
CACAATGTTGCCCAGGCTGGTCTTGAATTACTTGGCTCGAGTGATCCTCCCACCTTGGGCTCCAAAAGTG
CTGGGACTGTAGGCATGAGCTACTGCACCCAGTCAGGCCCTTGTTTTCTTTTGTGGCTGGAACCGTTTTC
TTACTCGAAGGTGCCTTTTTTTTTTTTTTTTTGACAAGCTAATTGCAAATAGTTCATACTGAGGTGCTGC
AGATAGTGTCAAGTTGCACGCTACTCGGAATATCTTCTGTGACTCAAAGTTATGTTACCCTTCATTTCTC
CCCCAGTGAACATAGCAAGCAACTTCTACTTTTCATTTGCTTCCTACACATATTACTTGCCTTTGTGATT
CCCTTTTTTAGCCTTGCCATCTCTGGCCTTTAGTAGGTTGAGGGGAGCTCCTGCTGTTGCTGACCAGAGA
TTTAGGGGGTGTACCTCAGGGTATTTTCATTAGAAAAACAGATGGCACACACTTCCTGAGATCTGTAGAC
AGAGTTGCTTTTTCATACACCTAAGTTGCACTGATTATTTATACCCTTTCTACCTACATTATGGTTCAGG
TTGTAAATTTCTCTTAACTTTTTTCAGAGTTTGGGATTTTATTTCTTGTTGCTTTTGGGTGAATTCCCAA
AGGAGAAGAGGGAAATGCTAACCTTATGCTACCATATGTGACCACAGTCATGCATATGTTTTGTGCTGGA
TTTTTTTTGCAGTTTGAATATGATGTACATGTATATAGTTTGTTATTTATCCTGCTTGGTATTTTCTGAG
CTTCCTGAATCTGTAGTTTGGTGTCTGTCTTTAGTTTTGGAAAATGCTCAGCCATTATTACTTCAAATCT
TTCTTTTGTTCCTTTCCTTCTTCTTCTGGTGTTCCCATTATGATTCCAAATGTTACACCTTTTAAGATTG
TCCCACTGTTCTTAGTTATTCCTTTATCTTTTTCATTCTTTTTTCTGTTTACTTTTCAGTTTGAGAACTT
TCTATTAATATATCTTCAAATGTACTGATTCTTTCTTCAGCTGTGTTTAATCTGCTTATGAGCCCATCAG
AGGCATTGTTCATTTCTGTTACCATGTTGATTTCTGGCATTTCCCTTTGGTTCTTTCTTAGAGTTTTCAT
CTCTCCATTTACATTTACCCATCTGTTCTTGCATGTTGTCCACTTTTTCCATTAAATCCTTTGACATATT
GATCATAATCATTTTAAATTCCAAGTCTGACAATTTCAAAATCTGTGCCATATCTTGAGTGTGGTCCTGG
TGCTTGCTTTGTCTCTTCAGACTGTGTTTTTTGCTTTTTAGCATGTTTATAATATTTTGCTGATAGCTAG
ACATGATATATCAGGTAAAAGGAAATGAGATAAATGATCCTTTAATGTGAGGTTTTATGTTTATCTGTCT
AGGAGTTACCGTTTGTAGCTGTAGGTGTCAAAGGCTAAAATTTCCTTTAGTGTCCTGTTTTTGTCTCTCT
TGTTGTCTCTGGGTTTCCCTAGAGACTGCTTTTTAATAAGAATCTGAGGGACCTCTTTCAACTATAACGC
CCAGGATCCTGACTAATGTAGTGGTAAGGTAAGGGGAGGTGAAGCGTTCTGGAGTCCTGTGCATAGTTCT
CCTTCTTTTAGTAAGCCTGTGTCCTAGGCTGTGACCTTCATAAGTGCTTCTCAGCCCACCACCCCTTGGG
TGAGACATGAAGGCTAGAGAGGGGTGGAGTTGAGTATTTCCACAGGTAAGTTAGGTTCTGGTAAAACCTC
AGTTGGTTAGGTTCTGGCAAAATTAGTTTCTCTTGAGGAAAGGATTTTGTTAAGAAGACCAGAACGCTTT
CCTCATTTATTTTGAAATGGGGAGTATTTCTAATTGGGGAGTATTTCAAAATGGCTACTTTTTCCCTCCG
CCTGTCAGAAGCAGGAAGGAATTTTTGTGTAATCTTTACCCTGAGACCTGGGGGGGCTCCTGGAGATAAA
ACTCACAAAAGTTTGTGAGTCCCTCTGAAACTGGGCCCTTGGAGTTTTTAACTCTGAAGCTTATCCACAC
GGATCCTCCATCAGTTCCTCAGTCACAGTTAATTGCTCCTACCAGTATTGGCTCCAGCTGTGAGCTTCTG
CTCCTGGCCTTTCTGTCCCCAGTAATTCTTGTGATTTTCTCAATCTGCCTCTCTCTCTCTTTAGTTTTCA
GGACAGCAGTTTGCCCTGTGACATTTGTTCTTTGATGTATCTAAGAAGAGTTACTGAGTTTCAGTTTGTT
CAACAGCTTTTTTCTTCTGGGGACTGTAGTGATGATTTCCAAGCTCTTCACATGTTAGATTGGAAACCAG
AAGTCTCTGTCTTATACATATAACTGATCTTTCCCTATAGTTCTTCCAGCAGCCATTCCAAATGAGGTGT
TATTTACAGATATATATGAGGAAAACAGTTGTTTCTCTTCTATTCAAAACGAAATATAGGATATCGACCA
CATAATCAGTGATAATATCTATCACCATTTCCTATGCCAAAAAAGAATTCTGTGGATATCACAAGGGCCT
GGTTTTATTTTTTAGCTTTCCGTTGTTGGCCTTTTGTTAAAATACTTGTTTTAGAAGATAAGTGACAGAC
TTAATTTTATTTTGTATTGAGGTTTTTAAAAAAAAAAACTTACGGCAATAGCCCATGTCTACTTGGACAG
TTTTCTTTTGCTGCATTGGTCTGTTAAAGATATTTCTTCCTTCCTCATCAGAATTAAAGAGCCCTCCTTT
TATAAACTTATTCCATTAAAGAAGAAAAGTAAATGCCTTTCAAACTGTTTTCCATAAACAGTATGTAATG
AGAAATACTGTAGCAGTATTTTCTTTTACTATTCGTAGTATCATTTTAAGGCATTAAAAAGCATTATTTG
TTCTTTAGAATGGATATACAGTGCAGATCTGAGTTATGTAATCTCTAGCCTTGGGAAAGAAAAAGATTGG
TGTGTTTTAATTTGTTATGTGCTCTTTGTATCTTTTCAGGCTATTGGCATGGCTGGGGTTGTTAAGATTT
TGTATTTGATTTTTTGAGAAAATAAAAGACAAATGACAATAAAACAAAGGAAATGGGTTGTATGGGTTTA
ATGTCCAGAAATACATCATCTTAAACTCTTCCCTTAATTATTACTATCAGTGGAATCTGGTACAATCAGT
TAAATGTATAAAATGAGGAAATGGAAAGTTTTAAGCACAATAGTAGACTTTATGAAATTGTACTGCCCTT
ATGTTTTCTTTCTGATCTGACACAGGTCAGTTGTGTCACTCATTAAACTCTAGCTATAGAAAGTAGAAGT
TGGTACCAATGACTACTAGGCCACTTGAACCTCTAAACTTAAAAATTACAGCTTTCCTTACTTGCCAGAC
GATAGCTTATTCTGTATGATGTTTTCAAAACTCTTTCAGTCACAGGTAAAATTAAGCAAAGACTGAAAAC
ATTCAGATCAAGCAAAACAATCAAGGCATAATCAACCATGAAATAACACTATATCAAGCTAAAGTTACTC
TTTTTCAATAGAAGAAAAAAATAAAATACTTAATGCTAAACACTGTTAGTTTTCAGTTAATCTATACTGA
CAACATTACTACTTTCCTCTAGGTTAGGGGTGAGCAAAATTTTTCTATAAAGAGCAAGATTTATAATATA
TTTATTACTAATGTATTTTATGTATATTTCACATACATAGTTTCTGTTACATATTTTAAATATATTTTTT
AAATGACTTAAATTTTAATATGGTTCTTAGCTCCGGGGCTGTAGAAAACCAGCCTTGGGCTGGGTTTTGG
CAGTGGCTTGCTGTCCCCTGCTATAGATGATACATGGGAAAATACTGCTTAAACATACTGCACCCAGGAC
CCCAAATAGGATTCAGAAAACATTGGTCATTTCTTGAAAGAAAGGGGCTTTTGTATTGTATCAAACGTCA
TCTGTGCACCTTATTAAAAATACTACAGCTCAGATAAGAACAAGAAAACAATGCTGTATATGTGTATGAA
ATTTGTCTAAACCACAAACTACTTTTTAAAGCTGAATAACGTATATGGCAAGTTTTTGAAAGAACAGAAA
TGCTTAACTTGGAGACCATGGAGTTGATAGGCTCTTTAAAGAGAGTTTATGTGGCCCCCAAATTTGTATA
TGAAATTTTAGATACTATACTTTTCTGTGGAGTACGTCTGTAAGATTTATATGACCCCAAAGATTAATAA
CCCACTGTTGAAGTGCTTTTAATCAGTCACCCCACACATGAAAAATTTATTAATGCATCTCTTGGTTTTT
GTCAGTTAATGACAAGCAGTGTAGTGAGGTTTTGTAGGAGTGAAGGCCGAGTCAGGCAGTAATAGATGGT
CATAGGAGGCTGTCAGCCTTTGTCTAGCGATTGAATAGCCATGTACTTGTAAGCAGCACAGTTGCCGATA
GGTGTGCTGCAGACAAACAAAAGCAAAAGCACGTACAGAAGAGACCAGGTTCGTATCTTGGATTTAAGTC
ACAAAGCAGAGGAATAGCAAGAACAGGGGATCAGGTTTAAGGTAGCTCAAACAAACTTCTGCATGGGTTT
GGTTACGGTCACTGCCAACTTTTTGGTACTGAGTCATACACGAGATGGTTCTCATGTCATGTTTGTATTC
TTCAGGCCACCTTGAATTCTTTCTAATATTGCATTAGACAATTGAACTGGATTTTTTTTTCTTCGATTAT
AGTAAAGTACACATGATATAAAATTTACCATTTGCCATTTTAACTATTTTTAAGTGTACAGTTTGGTAGC
ATTAAATATATTCATATTGTGCTACCATCACTACCATCTATCAATAGCACTCTTCCTCTTACAAAACTGA
AACCGTACTCCTTAAACAGTAAGTCCATTTCCATCCTCCCAGCCCCCTTGCCACCATCATTCTACTTTCT
GTTTATGAGTTGGACTATTCTAGGCACCTCATATCAGAAGTAGCATAGTATTTTTCTTTTTCTGTCTAAC
CTAATGTCCTCAAGGTTTATCCATGTTCTAGAATGCGTCAGAACTTCCTTTTTAAGGCTGAATTATATAA
GTATAATATATAATATAATTTTGTTTATCCATTTATCAGTTGATGGACAGTTGGGTTGCATCCACATTTT
AACTATTGTGAATAATGCTGCTATGAACATGGATGTACAAATACCTCTTCAGGTCCCTGCTCTCGACTCT
TTTAGATATGTACCCAGAAGTGGAATTGCTGGATCATATGGTAGTCCTTTTAAAAAATTTTTTGGGTAAC
TGTCATACTGTTTTCCATAGTGGTGCTATAATTTTACATTCCCACCACTAGTGCACACAGGTTCCAATTT
ATCCATATCCTCACCAACACTTATTTTGTGTTTTTTTGATAGCAGCCATCTGAATGAGTGTGGGTTGATG
AACCGGGTTTTTAAAGAAAATCTGAAGTGCTGGAGAATTTCTTGTAGTCTAGACACAGTAAATAACCACG
CAGATGGTCAAATTGCTCTTTCAGCATGCCAACAGAAGGAATAATTAGTTAGTTGCCTGTAGCAAATGCC
AAGTATCAGTAATTTTTCTGTGTATAGTATTAAATTTGATTTTTAAAAATGTTTAGGAATTTTCTCCCAG
CATAAAATTAAATGTTTTTAATTAGTTTATGTTAGTTGTATTTTTGAAAGTCTATTTTAGACAAGATTTG
AGTTTTATATGGGTGTGTAATACTTCCCCTACCATAGGCAGAACAGGTTTTTCACTGAGTTCATTAGTGA
ACTACTAAACATTCACAAAGCCTATTTGGAGATTTGGAGGATAATAAATACAACTATTAACAATTGTAGA
AGAGCATTTTAAGAGACAGTGTGGGCTTAAAGTCAGACAAATTCAGTTGACATGTAGCACTGCCACTTTC
TAGCTATCTGATCTTGGGCAGCTGAGCTTACATAAAATGAACAGTAATAATAATGACCCCTAAAAATGTT
ACGATAAATGTAAAATCTTTTAAAAATCTAAGAAATGTTTAGAGAAGTCTTCCGTCTTTAAGAAACTAAG
CATTATTTTGATTTTTTAAAACTGGCATTTATAGTAGGTATTTTCTTTTTGAATAATTGTTTCTGTAAAT
AACTGTTTTACTTTTTATCAAAAGGCATGGCTTTGGGAAGAAGACAAGAATTTGAGATCTGGCGTGAGGA
AATATGGAGAGGGAAACTGGTCTAAAATACTGTTGCATTATAAATTCAACAACCGGACAAGTGTCATGTT
AAAAGACAGATGGAGGACCATGAAGAAACTAAAACTGATTTCCTCAGACAGCGAAGACTGATTGTGTTTG
TAAAAGCTTGATGAAAGGACAGTTAAGTATTTTGATCACTGCATTTTGTTTGAAACTTGTGTCATTGATG
TAATTTAAAACTTTTGTTTAAAGCATTACAGTATTTTTCTGTGACCATCAATTAATGAGGGTTTGTGCTA
CCAGAGTTAAAGCATATGCTATCATTGTATTCTTTAAGAACCTTATTTTGATAAAATGTAAATTTGTTGA
ACCCTGCCACATTTAGTATCCCCACCCCCAAATCCTGTTCCAATGAAAAAATTAAAACCTGATACGAAAA
AAAAAAAATTCCAGTTAACCTATTTTGTGTCTGTAGGCTGACCTCAACCCTGTAACGTAACCCATTAAAA
TGAATTTCTTTTTTTTTAAGACAGAGTTTCTCTCTGTTGCCCAGGCTGGAGTGCAGTGGCGCAATTTCAG
CTCACTGCAACCTCTGCCTCCCAGGTTCAAGTGATTCTCCTGCCTCAGCCTCCTGAGTAGCTGGGATTAC
AGGCACACACCACCACGCCCAGCTAATTTTTGTATTTTTAGTAGAGGCGGGGTTTCACCATGCTGGTCAG
GATGTTCTCCAACTCCTGACTTCATGATCCACCCACCTCGGCCTCCCAAAGTGCTGAGATTACAGACGTG
AGCCACTGCGTCCTGCCTAAAATGAATTTTCTAGATGATTGAATAACAGTAGTCCTTTGATAGAAGATAA
TGACTTGGTTTATGGCCTTAATATACTACTTAATTACTTAAGATGTTTATTAATAGAATGATAAATGTAC
AGAGTAACCTATAAGCATGACATACTTTTGCTTTCAGTAGTTTCATGTAAAGAAAAAAACTTGAAAATAG
TAATACCTGAGTACCCATGGGAATAATAGACACTGGGGAGGTAGGGTGGGGAGCGGGAGCAAGAGCTGAA
AAACTTACCTACTGGGGACTGTGCTCACTACCTGGGTGACAGGATCATACGTACCCCAAACCTCAACATC
ACACAGTATACTCAGCTAACAAACCTGCCCATGTGTTTCCTGAATCTAAAATAAAAATCGAAATAATTTT
TTTAAAAAAGAAAAAGACAATAGTATTACCCATGGGACAAAATTTGTACTATTAGCAAGAATCATTTTGT
GTCTCATTTAGAAACAATTTGACTTTTGTTCCAGTGTTTAAACTTTGACAAAAATGGTTTTGAATAGATC
TTTATAACCTGATGCCATAAATACAAGATTCTCTGATACCTTCATTTAATATATCAATATTGGGCCTAAA
ACAGTATTCTGTAAAGCTTAAATTGGTATTAACTATGATCATCTTGATGTCTATGATAGATAATAAACAA
GGTCATACATACCTTACTAAACAATTTTGGTTTTTCACCAACATTTTATTCTTTAAAAGATTTAGACTAA
CAGAATTATTTAGCATTTCGAGTCATGTGCTTTATTTAGCAAGTGAGTAAAAATATTGGAATATTGAAGT
ATTTGCATAAAAAATCAAATGGTGGTGTTTTGTAATCTCTATTGTATTTCCTATTAAGGTTTCATATATT
ACTTTCCCATTGTTCCTGAATTTGTTATCCTATATATAAACAGAAACATGGATGAGTACCATATACTTCC
TGACTCCTTTATCTTGTTACACCACAAGATACTGCACTATTTATTGGAGATATTTTGATGATTAGTGCCA
AGGCTGGCATCATGAGCCAAAATAGACTTGTTAAGTCAACCTCATTCCAAACACCTGCTATAGATCATCA
AATCTAAGTTGCCATTAATTGTAAGATGTCCTGTTATTTCACACACACAACTAGAAAAGAAAAATCGGTA
TCAATTATAGTATGGTGCTTTCTTATTTTGAATTTTACTTATTAGAAGAGCTATTTTAGATGTATTTAAA
CATAAGATTTTTATCACATAACTTGTGGATACAAAAAAAGAGAAATTGTAAGCAAAATAAGTTGGGTAAT
ATATTCCTAAAACCTCATCTTCTCCAGTCTGAGTATTTTTTTGAATCCTTTTTTTTGGTTTTGTTTTGTT
TTGAGACAGGGTCTCACTTCATTGCCCAGCCTGGAGTGCAGTGGCACAATCACAGCTCACTGCAGCCTCT
ACTTCCTGGGCTCAGGTGATTGTCCTACCTTGGCCTCACAAGTAGCTGGGACTACAGGCATGTGCTGCCA
CTCCCAGCTAATTTTTGTATTTCTTTGTAGAAATAGGGCTTCTCCATGTTTCCCAGGTTGGTCACAAACT
CCTGGGCTCAAGAGATCCATCCACCTCAACCTCCCAAGGTGTTGGGATCACAGGCATGAGCCACTGAGCC
CTGGCCCCTGAATTCTTTTTAGTCAGAATTTCTGTTGAGGTAATGTAAATTCACATGCAATTGTAAGACA
TAATATAGAGATCTCTGTACACTTGGCCCTGTTTCCCCAAATATTAATATTTTGTAAAACTATAGTGTAT
CACCACCAGGATATTGATATTGATACAATCCATTCAACTTATTTCCCCAGTTTAGTATTCTGTGTATTTA
GATCTAAACAGTATTATCACTTGTGTTCATACATCCACCACCATGAGGATCCCTCACATTGCTCTTTTGT
AAGCACACATACCTCTCTTTCCCCAAATCCTGACAGCCACTAACATATCCTCTATTTATAAAAGTTAATC
ATTTTAAAATCTTATATAGTGACATCACCAAAAATAGCAAAATGGGGAGCTCCAAAGATCTGTCCCTCCA
CTGAAGCAACCATTGCTAGCAAAAACTTAAGATCAACTTTTTGGATCTCTACTAATAAAAACAACAACCA
GAGGAATGCTTAATGAAAAGGCTATTTAGCTTTGGTAAAAGAGTTTTTGTGGTTTTGCTTACGTGCCTAC
CATGCCCTGTCTCCCAGCCAGGCAACAGCTGAGAGGACAGTGGCCTGTGCTCCTAGTGCATCTTGATACC
AGAACGGGCAATACAGATCTTGCTCTCACAGAAATGTGGGTGTGCTTTGACCTGCCTGGCAGTTCCTGAA
GGTTCAGCTCAGAGGGTTGCCTTTGTTTTGCCCCCTCCACACTAGAACAGCCTCACAGTGCTTATCAGAA
GCATTACCTGCAGCCATCTGGGGCTGGGGACGGCAAACGGGGCAAGCAGCAAACAGACCAAAAAACTTGG
AAAGGAAATAACTGGGAGGAAGATACGTGGGAGAGAGAAGGCTCAGAAGGGCCCTTGTCTGTACTAGGTA
GTCCGGAAGGCCACACACATGCCTAACACTGGACACATTTTTGGAAAAGACCTGAGAGGACTCTAGGCCT
GCACCTCTGACTGATCTTTAGGCTCCACAGAAGTAGGAGGTGGAAGCTAAAGCAGAATTTAGGTGACCTG
GCTAAGCGCTGAAGGAATGCCCAAGTCAGGACGAATCTACAAAGCCTGGGAGAATTACCATTTTTAACTT
TTTATTTTATTTTATTTTTTTTAAATAGAGACATGGTCTCGCTTTGTTGCCTGGGCTTGTCTTTAATTCC
TGGCCTCAAGAAATCCTCCTGCCTCAACTTTTCAAAGTGAAACAGTATCTTTTGTTCTTTTGGACTCATG
ACAGTTAAGGAAATTAAGTGAAATGCTGTACGTATTCTTTGCCATCTTTCAATCAGCATGTTTGTACAAT
TCAAATTGTAGCTGTAGCTTGTTTATTTTTATTTCTACACTAATCCACTACATGATTCTATTAATAATTT
ACGCTGTTGATGAGCGTTTGAAGTGTAAAAATAATTGAAGACCAAACATGAGAAAAACAGACTTTTTATT
CAGAGCTTGCGGTAGCAAAGGAATCAGCCACGATCTCTTGTGTTTAGCAGACTCAGAGGCAGATAGGTTT
ATAGTGGGGGGAAAGGGGCAAGGTTCAGGAATGCTCTGCTTGGAGGGTGTTGGCATGAGGAAGCTGGAGG
CTGATCAGCTCACTGCAGGTGTCTTACAGGATTGCTTTGGGGAACCTATTTGGCTTTCTCTGATTGGTCC
CATGTTGGAAGAGGGAGCAAAATGTTGGGATGATGGCAGTAACTGAGTAAGTCTTGACCATTCTGAATGG
ATCACTGGGGAGATTGTGGTTTGGCTTCCAGGAGTGGTTGCTGCAGAGGGTCTGGGTGAGAGTTCTATTG
TCACAGAAGCTCGTCCATTGTCCGTGTGTATGCCTAGTCCCAGAGTTGTTTCCAGTTTCTGGCTGCTAGA
CTTTTACATTTCACAATGAAGTGTGATTTTAGAAATAATATGAACTCTTGCCTGCCAGAACAAGGGCCAG
TGAATTTTAAAGGAAGGAAGCACAATCTAGAACTCGACAGCAAGGGTCAACCATGAAAAGTTAGGTATGA
ATATTGTTAATTCCTAGAGCAAGAATTTTTTCAAAAATATAGATAGATAGCCAATAGAATACTAAATTAC
TCTATTTGGAATACTAATAAACCCAGAAACAAAAAGTAGTGAAACAAAAACAAAATCACAGGGACAAACA
GAAGAAATAAAATGGTACACCTAAATCCAACCACATCAATAATTATATCAAATGTTAACGGTCTAAGCCA
GAAGGTGCGTGGCAAACTTCTTCTGTAAAGAGCCAGATAGTACATATTTTAGGCTTTGCGGGCCATACAG
TCTGTATACTCTGCCTCAAATTAGCCATAGGCAATATATAAATGAGTGGGTATGGCTGTGTTCCAATAAA
ACTATATTTACAACAACAAACAGGCTGGATTTAGCCACAGGTTATAGTTTCCTGACCTTTGCTGTGGAAT
TCTAGAGTGTGTTTCCTTCCTTTAAAATTCATTGGACCTTGTTCTGGCAAGCAGTTAAATTACTTCTGAA
TCTGGGAACTTCTTTTAATTCTCTTGGCTTTTTTTCCAGTTTTGGAAGTTTCCAAGTATGGAACCACTTA
TAAATTAACAGGTTGGTTTTCCAAGATCAGCCTGGTGATGAGAATTTGGGCTACAAATAGACAGGATAGC
TGACTGCCAATGACCTCTTAGTCCCATTCAGTGCTGCCCAGTTCCTTGAGCCTTAAGGGAGGGTTTGAGG
TGACTTCTGGTTCAGTCTCCCCCTAAGAGTATAGTCTTTCAGTGTCCTCATTTAATTATTCCCATCTCAA
TGTGTCCTTGGGCTTTGACTTCTGGCCCCCTTGCTGAGGAAGAGAAGACAATAATGGAAGTAAGAAGTAT
AGGACCCCCTTTCCTCCAAGGCTAAGATTCAGACCTCTTCAAAGAGAGGACCTTCCTACACCTGGACCAC
AGCCCACAGCGGGGAAGAAACTGGCCAGAGGGCACAGGTGGTGCTAACCTGGAACAGCAGCCCAGGAGGG
TGTGGCTGGGTGGAAAGGTGGCCTGGGGGCAACTGGAACTCATCCACATCACTCCCCCCCTGAATAATAA
TGATAGGACCAGGACTGGAAGGGAATGGTCTTCCTGTCCTTTCACTCCAGAGACCCTCCAAAGCCCTCTG
TTGGTAAAGCTTAATGATCATTGTGTCAACAGATGTTTACAGAGTCCAGCCCATGTCCATCTATCACAAA
GCAGGGCAGAGAAGGGTGGATTTGGAGCTGAGAGCCATTATATTGATAACTGGCACATAAAGTGCTTTTA
TTTTTTTCAACTTACTCCCAACACACACACACACACACACACACACACACACACACACACGCACGCACGC
ACACCCCTTCATATGTCAAAATGACATTAAGTTTCATTTAAATGTATCCTATATTGAAAGAGCAAAAAAA
AAAAGTGACAGTTTGACTCAAGAAATGCAATGGTTTATATGACCTACAACTCTCGAAGGAAAAGAGTAAT
AATTTTGCAATGTTTTGATGATATTTACAAGTCATAATTTAAAGAGATACATAATTTTTCCCTTTCTACG
TAAGTCCTCAAGGAACTCACATCTGTTTTAGCCCAGATTTGTATAGTGTTTTGCTTTGCATGGATTATCT
CATTTAATTCTTGCAGAACTTTGATGAATGGTATTATTAATTAGCAGAATGGTAGTAGTAACAGGCAAAG
TGGTATTGTTAATTGCATGTAATAGATAAGGAATCAAGCTTCAGTTATTTTGACTTGCCCAAAATTGATT
TGGTAAGTGACAGAACTTTATTTGCTGATAGTCCACAATTAAAACTTTTCATTATTCAGGTAGAATTCTT
TTCCCTTTTATTCTATACATGTTGATATGCTTGTTATAAAGAAGCCTAGGCCTGTGATCTCGATACCCGT
GGAGCTCTGCAGCTGCAGCAGATAGTAGAATGTATGGGAATTCACCCAAATGCACCTACAGTAGAAGTAT
TATTCATTATTTCAGAGATGCTTTTTCCTCTGTGTCTGGTTATAGATCAGTTGGCATGGCATCAAGCAGG
TTGATGTTCCTCTTATCCCAAGCCCCAAGCCTTACTTTTTCTTTTTTTTGTTTTTGTTTTTTTGAGACAG
TCTCACACTGTCGCCCAGGCTGGACTGCAGTGGCACGATCTCTGCTCACTGCACCCTCTGACTGCTGGAT
TCAAGTGATTCTCCTCCTCAGCCTCCCAAGTAACTGGGACCACAGGTGCTTGCCACCACACCCAGCTAAT
TTTTTTGTATTTTTAGTGGAGACAGGGTCTTGCCATGTTGCCCAGGCTGGTCTTGAACTCCTGGCCTCAA
GCCATCGTCTCACCTCGGCCTCCCAAAGTGCTGGGATTACAGGCATGAGCCACCACGCCCAGCCATTCCA
GCCCTTTTTACTTTTACCAAGTGGTTACCACAACAACTTTGATGAGCTGTTATTCAAAGGAATCCTGTGA
TGAGCAATTTTGAAAAGAAAAATGATCTCTGTATTATATATTTGGAATTTTGAATAGGAAGTAGGTTAAA
CATGTGTTTAACACTTAACACATCCCAGGGGCCTCTCAATTGTGGCACAGTGTGACCCCTACATGCTGTA
TGTGTATGAAGCACAGCAGAAGCACAGAGGCAAAACACACCTCAGTCGGCTTCCCTTGAGTTCCCTTTTA
AAGAGTCTTTTCTATATTTCTGGATCATTCCTTTCTTTTTTATGTTTTTTTTGGTTTTGTTTTGTTTTGA
GACAGGGTCTTGCTCTGTCACCCAGGTTGGAGTGCAGTGGTGCAATGACTCACTGCAACCTCTGCCTCCT
GGACTCAAGCAATCCACCCACCTTGGCCTCCCCGAGTGCTGGGATTACAGGAGTGAGCCACCATGCCCAG
CTTTTTAATTTTTTTTTTTTGAAACCTATTACAGTAACTCCCTGAGATGAAGATGAGACGTTTAGCTTCC
TCACACTCCCTCCTCCTCCTCCTTCCCCATGCACCTCCCATGCTTTGTTCAATGTTACTCTCCTTTTACT
TGCTTAAGGTACTCTTTCTTATGCCATTGACTTTAGACGGAATTCCTTGGCTTTACTGAGTAAGATGGGG
AAATTAGAAATCTGCAGTC

An exemplary DNA sequence is human Topbp1 provided below.

gi|224589815: c133389958-133310251 Homo sapiens chromosome 3,
GRCh37.p13 Primary Assembly
(SEQ ID NO: 310)
ATACTTGATATATTTATTGGAGCCTCTTCAAGGTGTGGCATTATCTTTTTATCTCCCATTCCATGGAACT
TCAAATTGTGCCAAGTATCTTAAGGAGAAAACTGGCCCTTTGTCAAGCTTAGTTCTCTGCTCCTCCCTTC
TCAATGGGATCTTGCCTCCACTTCTGTTTTTCCAGCCCTGAGACTGCCATCAGTTCGGCTGGCTTCTCTG
TAAGCCAGCAGTGACCCTCTGCTCAGGTAAAGCCAGATTCCCCCTGGCCATGTGCCGAGTGGTTCATCTT
TAACTGTCCTGGCTCTCCCTTCACTTACTCATACATTTGAAAGGAAATAGGATCTATGAGCGCAGAGATC
ACTTTTTTCTTGTCCAACATTCTAACACAAGGTTGGAAAATACTGTCTGCTATAGTATTGGGCTCAAAAA
ATCAAACAGATCAATAAATGCACACAAATGTCTCAAAAGTGGGTTGAGCAATCTCCTATGCAGGCCTATG
GACAAAAACCAAATAAAGTCCAGAGGTGATCATTCCTTCTAAGGTTAATTATTCACCTAAAACTAACACA
GCCTAAATCCAATAGCAGCCACTGTGCCCCCGACCCTAGATTAGATCAAACTCTGAAAACTTCGTGTGTA
CTGGGACAAAGTGATATTGCAAAGATAGGACATCCAAAGTGCTAAAGTCATTTCTGTGCTTTGGTACAGG
CTCTCATAAATAGCAAAGCAGAAGGTTAGATGCTTGTATGGTGGAAAACAGTTTTAAAACTTCACGGCAA
AGATGGTCTTTGGAAAACTTGGGATTTATAGATTTATTAGTGTGTACCAGGCATTGTACTAAGTGCTTCC
CAAATGTTAAATGATAGTAACCCCATAAGGCTATTGCCATCCTCTTTTTGTACAGAGAGACAGTGAAGCA
CAGAAAGGTTAACCAGTGACTTGCCCAAGGTCAGGTAGCTAGTAGGTGTCCAAGCTGGCATTCAAACTAG
GCAGCCTGCTTCCATAGTCCATGCTGTTTCTCATGTGGCCTCTGACATTTGTCTCTGGAGCAATTTCACA
CTTTCCCCTTTCCAAGGCATCTTGAAGTAATAACGGCATGGGGCACCATGTTTGTTTAAACTGTTTCCAA
GTTCAGAATTGGTTGGATTTAACTGTCCTGGCTCTCCCTTCACTTATTCATACATTTGAAAGGAAATAGG
GTCTATGAGGGCAGAGATCAAATTTTCTTGCCCAACATTCTAATATAAGGTTGGAAAATAGTGTCTCACA
AATAGCACTAACTGCTATAATAGCAAAGCTCCAAAATCTTAATGGTTTAACACAATAGGAGTTGACCTCT
TAGTAACATAAAGCCCATCTGGCAGCAATCGTGGTGATTTGTTAGGGTGGGAGTGGGGAGTTCACTTTCA
TAGCTCCATTCAGTCATTCAGAGATCCAGGCTGAGAGAGGTTCTGCCATTTTCAACACATGGCTTCCAAG
ATTGCTCTGTGTGTTGATATTCAAATGACAGAATAAGGAAGAGCGTGGAAGGTAGTGCAAGAAATTTCCT
TGGATCAAGCTCGGATGTGACAAACATCACTTCTACCTATATTCTATTGGCCATAACTCAGTCCCATGAC
CCCACCAATTGTAAAGAGGATGGGAACCATAGTCTCTGGCTAGGAAACTACTTCTCAGCAACAGCAAAAC
ACTATGAAAGGGGAGCACATGTCTGGGGTCTTTTGTCTTAGTTTGAGCAGACCCAGAAAACAGACTTTTA
GACAAGCCTTCTCAGATGTTTTATTTGACAAGTAAAGAAACACAGGCAAGACAGTAGAGATGTGGCTGGG
CACGGTGGCTTATGCTTGCAATCCCAGCACTTTGGGAGGCCTAGGTGGGTGGATCGCCTGAGGTCAGGAG
TTTGAGACCAGCCTGGCCAATATAGTGAAACCCCGTCTCTACTAAAAATACAAAAAATTAGCCAGGTGTG
GTCGCAGGCACCTGTAATCCCAGCTACTCGGGAGGTCAAGGCAAGAAAATCGCTTGAACCCGGGAGGCGG
AGGTTGCAGTGAACTGAGATCGCGCCATTGCACTCCACCCTGGACAACAGAGCAAAACTCCGTCTCAGAA
AAAAAAAGAAAAAAGAAAATAGAGATGTGAGACAGGGAGAAAGGCATCCAAAGAATGTGTTAACAAGTCA
GATGCCACTGTGGACACTGGGACTTAATTCCCCTGAGGAAACTATGAGAGTCAGAGTGGATGTCCCATCC
ACGGGACATGCAAACAAGGGTTTTTGTGCACCAACTCCTGTCAGCCATCATTTAAGGGGTTTTTTTCCCT
ATAGGTCATAAATCCTCTAGCCCTCTGGCCTACTGTGCAGGTGGCAGAGCAGATTCCTGGTGCCAGAGAA
TGCCTTCATGCAATGAACACAGATGCTGGCAGCTGGAAGTTTCCTAATGTGCATGAGTGCTAAGGGTGAG
GGGGTATGGGTGGGACACTAAGGGTTTCTACATGGTCTCTGCCAGAATCCAGACATGTTTTTTTCCCTCC
CTGCCCAAGCACACATCTGCTACACACTGGGGAATGGGGAGGCACAAACAGAATAACAACAATAAAACTC
CCTTTCAGAAAAGGGAGGAGTGGAGACCCATAGCCATCATTGGTCAGTATCAATTTTGAAGTTGGGCAGG
CACTAGGAGGCTCTCGATTGGGGGTTCTGATTAGATCAGATTCTGTTCTGTGACAGGAGCTCCTTTGCTC
GTTGCTCTCCAGGATCCCTGGTACCTCCTTCTGGGAGGATCTTCATTTTCTATTACCCTCTCTATCCATA
TCTGAATACTGAGGAGTTTGTGCTCCTTGGGGCTGTATGACTTTCCACCTACTTGTGCTGCGAAAATTTG
AGGATTAAATCAAAGTCTGAATACTCCAGGTTTCTGGTTGGGTTCAGAAAGCAATTATCTCAAAATGTTC
GTAACCTTCAGATCTATTTGGTTCCAGTCAGTTTCACGTGGTAATTGTCAACCTCACACATACAAAACAA
GGTCAAGGGTAGGGCCTGATTCCACCAGTGAACATACTTGAGACATTTTTAGATTTAGATGGTTTATTAC
TTCCATAGATAGCAACAGAGAAGAATAAGTGAAAGATGTCTTCTCCCTGGGTCTGATGATTGCAATGCGA
GTTGTAGGATACTCTATTACTAAGGAGTCAAATCTAGATGGCAGCTAGTGCTTTTATATTCTGCAGGCCT
ATCATGAGGGGAGTGGGGCAGAAAGCCCCATATCTCATCAAAACCTGGGAGGTGATGAGAAACTATGTCA
TGACAGCCTCTTAGGAGAGATAGAAAGGTGAGTGAGAGATGCCTTTCTAGTAGCTCCTTAGACACCTCCC
TTAAGGATGACAAGGTGTTGTGCCAGGGCTCAGATAAGTTGCAATTCAAGCCTTTGCCTCTATTGATATG
TGTAAGCTTATCAATAGAGGAAGTCATGGCAGAGCTGTTCTTCTACAGAAGTAGTCCCCTCCCTAGTTCT
TTCCTAGACATAATTTTCAAACCTGATTGCTATCTTTGGCCCCATACCTCTCTCTCTCTCTCTCAGTTGA
TGGTGCCTACCTAGCTTGGGTGACAGGACCATATTCTTTAACTAGATCTTTTCCACATGGTCGGGTTGTA
ATGGACATTTGTTGCTCAAAACCTTTCTCAGGTTTATCTCTTGTTTTTAGGACACAGATGAAGTCAGCTT
TTCCAACCTTGCAAAATCTCACATACTTGGGTTTTTTTTTTTTTTTTTGAGTCAGAGTCTTGCTCTGTCA
CTCAGGCTAGAGTGCAATAGTGAGATCATAGCTCACTGTAACCTCAAACTCCTGGGCTCAAGCAATCCTC
CCACCTCAGCCTCCCAGTACCTGGGACTACAGGTGTGTGTCCTCCACACTCAGTTAATTTTAAAAAAAAA
CAACAAAACTTTTTTTTAGAGACAAGATCTGGCTGTGTCGCCCAAGCTGGTCTTGAATTCTTGGCCTCAA
GCAATTCTCCTGCCTCAGCCTTCCAAAGTGCTAGGATTACAGGTGTGAGCCACTGCATCCGGCCTGGATT
TTTTTTTTTTTCTTTTTCTGAGACAGTGTCTCGCTCTGCTGCCCAGGTTGGAGTGCAGTGGCACGATCTC
AGCTCACTGCAAGCTCCACCTCCCAGGTTCACGCCATTGTCCTGCCTCAGCCTCCCGAGTAGCTGGGACT
ACAGGCGCCCGCCACCACGCCTGGCTAATTTTTTGTATTTTTAGTAGAGACGAGGTTTCACTGTGTTAGC
CAGGATGGTCTCGATCTTCTGACCTCATGATCTACCTGCCTCAGCCTCCCAAAATGCTGGGATTACAGGC
ATGAGCCACCATGCCTGGCCAGGCCTGAATTTTTAAATTCTCTTTATTTTTGTTTGCAAACAGGCCAATT
CCCTGAGCTTGTCTTTTGCTTATCTAATTAAATGCAGCCAGTAGCAACCCACATAGAATAGAATGTGCTA
ATGACATTCTATTTTACCTCCTCTTTGCTAGAGGAGACTTAGTAGGCCTATGTTCTATCTTCCAGGTTAC
TGAAGACAATAGTTTAAACAAATGTTTTGCTAATCAGACTATTTCTGTAGCGATAATATCATTTGCTAAT
TTCACTTCTAAATTGTACATTCAAGGCCGGGTGCGGTGGCTCATACCTGTAATCCCAGCACTTTGGGAGG
TCGAGGTGGGTGAATCACAAGATCAGGAGTTCAAGACCAGCCTGGCCAATATGGTGAAACCCCATCTCTA
CTAAAAATACAAAAATTAGCCAGGCATGGTGGCACATGCCTGTAGTCCCAGCCACTCAAGAGGCTGAGGC
AGAAGAATCGCTTGAACCCAGGAGGCGGAGGTTGCAGTGAGCCGAGATCATGCCACTGCACTCTAACCTG
GGCGACAGAGCGAGACTCCATCTCAAAACAAAAACCAAAGCAAATTGTACATTCATTTATTTATACGTAT
TTATTCATTTACAGCATTGGAAACAAACATAAAACTCTATAACTCAAAAAACCTATTTTTAGTTTGGCTT
TAATTTTGAGAAATATTATCTATGTAGTAAAATATAATAATAAATATTCATATTCCCACCACCCACATTG
TAAAAGTAACTATTTTGTCATATTTGTTTCCTGTGTTTTCTTTCCCGTTCTTTTTTTTTTTTTTTTTTTG
AGATGGAGTTTCGCTCTTTTTGCCCAGGCTGGAGTGCAATGGTGCAATCTCGGCTCACCACAACCTCCGC
CTCCTGGGTTCAAGCAATTCTCCTATCTCATCCTCCCGAGTACAGGCGCCTGCCACCATGCCTGGCTAAT
TTTTTTTATTTTTAGTAGAGATGGGTTTCTTCATGTTGGTCAGGCTGGTCTCGAACTCCCGATCTCAGAT
GATCTGCCCACCTCGGCCTCCCAAAGTGCTGGGATTACAGGCATGAGCCACCACTCCCGGCCTCCTTTTC
CATTCTATAAACAGATAAAGTTGCCCATAATGTCACCCCAAGCAGGGTCTGGAGAGACTGGCTCTAGGTG
ACAGAAGTGCCAGAGAAACAGGAAACTGATCCTAAATGCCAGGAAAGTGAGGGAAAAGTTAATTCCCTAC
CCCAACTACATTTTTTTTTCTTTTTCTTTTTTCTTTTTTTTTTTTTTGAGATAGGGTCTCACTCTGTTAT
CCCAGGCTGGAATGCAGTGGCATGATCATGACTCACCACAGCCTTGACCTCCTAGGCTCAGGTGATCCTC
CCACCTCAGCCTCCTGAGTAGCTGAGACTACAGATGCCCGCCACCACGTTCAGCTCATTTTTGTATTTTT
TGTAGAGATGAGGTTTCACCATGTTGCCCAGGTTGGTCTCAAAGTCTGGGGCTCAAGCAATCTGCCCACC
TTGGCCTTCCAAAGTACTAGGATTACAGATGTGAGCCATAGCACCCGGTCCCCAGCTTCTTTTTTAAAAA
AGTTTGTAAATATTAGAGATGGGGGTTCTTGCTACGTTGCCTAGGCTGGTCTCGAACTCTCAGCCTCAAG
TGATCCTCCAGCCTGGGCCTCCTAAAGTACTAGAATTACAGGCGAGAGTCACCACGACCGGCAAATATGG
AATGCTTCACAAATTTGCATGTCATCCTTGCACATGGGCAATGCTTATCTCTGTATCATTCCATTTTTTT
TTCAGTAAACATGCTGCCAAAGTAAGCACCGAAACCTCAGCTTCCGAGGGCATATCATAACCTTTCCCTG
TTTTCACCAAAGTCTTCAGGGACCACAAACCCATATACAGTTAGTTCCTTGTATCTGTGGGTTCCACATC
TGAGGAGGTCAACCAACCGGGGATCAAAAATATTCAGAAAAAAAATACAATTTTTATGTAAAATTTGCCA
TAGTAGAAAAAATACAACAATAAAAAAACAAAATTTAAAAATACAGTATAACAAACGTTTACATAGTATT
TACATAGCATCAGGTATTATAAGTAACCTAGTGATTATTTAAAGTACATGGGAGTGGCCAGGCGCAGTGA
CTTATGCCTGTAATCCCAGCACTTTGGGAGGCTGAGTCAGGTGGATCACGAGGTCAGGAGTTAAAGACCA
GCCTGGCCAACAAGGTGAAACCCGTCTCCACTAAAAATGCAAAAATTAGCCGGGTGCGATGGCAGACGCC
TGTAACCCCAGCTACTTGGGAGGCTGAGGCAGGAGAATCGCTTGAACCCCGGGGGTGGAGGTTGCAGTGA
GCAGACATTGTGCCAACTGCACTCCAACCTGGACAACAGTGAGACTCCGTCTCAAAAAAAAGAAAAAAAA
AGTACATGGGAGTATGTGCACAGGTTATATATAAGGTACTTGAGCATCTTCAGATTTTGTTATCTGTAGG
GGGCCTTGGAATCAATCCTCCGTGGATACTAACCATTTCACCCTTTCCTGAAGGACTGCTGTTCAAGCAA
ACTTTAATGTGTACCAAAATCACCTGAAGGGCACAGATTGCTAGCCCTACCCCAAGTGCTCCTGACTTAA
TAAGTCTGAAGTGGAGCCCAAGAATTTTCATTCCCCCAAAATTCCCAGGAGATGCCGATGCTGCTGGTCC
AGACACTACACATCAGAACCACTGCTTTATCCATTTTCCAGGAGTAGATACTAGAGACAGACCCTCTCTG
CTTATCTGGAGACACAAGATGGGAGAACTTGCCCCCTAGTGGTGAACTCTTACTCTAAAGACAGGTCACT
CCTCTGGTATCAAGTTCCGGCTGGGTAAAAGCTAAAGGGTGCATTTTCTTCATCTTCATTAGCAGCATCT
ATGAATCTCCTTCTCTTGTGAGGTCTCTACTTGGCCCAGAAATGCCAGGAATGGGCTCCCTCCCCGGTGT
GGAACTTCACACAGAGATGGGCAGACAGCACGTGCCCAGGAGCAGTGTGGTGTGTCAGGAAAGGCACGGG
CAAGTGACACAGTCCCTGAACTTCACTTTCCCCATCTCCATAACATGAGACAATGATACCCATCTATTAT
AGGGTTGCTGAAAAGAGTAAACGGGAATAGTGTGAGCAGGAGTCCCATGTTAACTGTAAAGTGCCCTAGA
AATATTAATTGTTATTACAGATTCAGGTCAGATTTGCACCTTAACAATTAACTGTGGTTTCTTTTTTTTC
CCACCTCTAGTGATTTTTGTTTTTTTGCTTTTAAGGCTTTCTTCACCCTTCCCCCAGCAATAGGCCTTTA
CCTTTGAGCCCCAGTGTTAATTGATTCTGACTCATTTCTGGTAAAAAAAAAAAAAAAAAAAAATTAATGA
TATTTATTAAAGCACAGTAAGACAGATTTTATTCCGGACCATCAGGATAGGTATAGCAACCATGGCAATG
GGATTCTGAATTAGGAGAGAGAGAGTGGGCTCAACTCTAAATACACCATGGGCAAGTGGGAATTTATAGC
CAAGGAACAAAAAGCTTGGGGTCACTGGATAAAAAATTACTAAGAAGGAACTTCAGGAGTAAAGGGGTTT
CTTGCTGAAAATAGGTCAAGGTGATCAGACATCACCTTGGATGGTAGAGGATGAGGAACCTGATCAGATA
TTGAGGGTAGAGGGTTCTAACTGGATTTTCCAAGGAAATGCACAGATAAACCTACGAGAAGGTTGAGGAG
TCTGACTAAAGTTTGGCCAAGCAAAGAATCTTTGTCACCTTCCATCGGTGTATTCAAAAACTTAAGACTC
AAGCTGCTCATTACTATCTTTAGGCTCTAAAAAGAAAAGAAAAACAAAAACAATCAAGGGACATGGCATT
ATACTTTTACATTAGTAAATCCAAAGTCATTTTCTCAATCTCAGCAGTGAATAATAAAATGATCCTCATT
AGTATAGTGGCTAGATTTTATAAAATAAGAAAAAATTTTTTTAAAAAAGAATAAAATGAAAAGAAGCAAC
AACGAATCAGTTTTCCGCTGGTTATCACTAAGGTTTGCAGGCCCATCTTATCCCCTCTCCCTTTGCTCTC
TGCCATCTGAATCTTAGGTTCAGTCCCAGAATCTCCCCAGTACTGAGGCAGATGCAGAGAAAGAGAACGG
CGATGTCCACTGAAGGGGAGGAGGAGGGGAGACCATTGAAGATCTTGACAAAGATCCCTTTGCATTAGCT
GTGTAATAAGTCATCCTTTCTTCCCTTGCCATCTTGTGGCTGTGTTAGGCGGTGATTCTATTCCTGCTGT
GTTGGAGGGAAACCACCCAGTCCGTAGTCAGAAGACCTGGATGCAGTATTAATTCTACCACCTACTAGCC
GTGTGCCCTTGAACCCATCATTTAACTTCCTCCGTTTTTACATCTACAAAATGGAACTACCACTATTTGC
CTCACGGAATTGTTTTGGATAATACCTTTTCCCGTGCATGCCTGGTATAAGACAGGTGTAAAATAAATAT
CTATTGAGGAAATCCTTTCTTTCCCTGGCTTTTTTAACAGTAAAGGAGTATTCCTGGCCTGTGGTCCTAC
CTCTTGAACCGACGGCGCTGGGCCTACTCGCAGCGGAGACTAGGCCCGCCCAGCAAAGCCCTAAACCCAA
AGGGTCCGGCCCAAGCTTTCCCTGAAGCCCCACCGCCCACAGACTGCTCACCTCCACGTTTGACATTTCC
GCTCTTCTGCTGCAGCTGCCGATGGCGTCACTTCCACCGGAAAAGGCAAGCGCGGCCGCGCGGTTGGCGC
GAAAGTCGGTTCCCGTTCTGGGCAATTTCCGGGTTCTCCAGCGTTTGCTTTGGGGTAGGGGCGGCGCCGA
GTCGGGGGAGGGGGCTGTGCGCCGGGCTGGCGCCCGACCCCAGCCACCGCCCTGCGGCCAGCGCGTCCCC
CGACTCGCCGCCCGGAGACCCCGAGGCTCCAACGAGGTAAGTGAGGCGCGGAGTGGGGACACAGCTCCTC
CCCAGCCCCAACTGGGGTTCGCGATGCCTTCTGTGGCCTTCAGGACCCTCTTTTCCTGTCCTGGTAACTG
GCCCTGGAGCCGGCGGGTGGGCCTTTGACGCCGAGAGGACGTGGCTTCCGTGATCCCAGAGCCAGGCCTT
TTCCTGGAGATGGGAACCGGAGCATCTAGCCTTATGCAGGGCCAAGACCATGCTAACTGGCTTCCATGTT
TATATTTAAAATCTGTTTACTGAGGGATGAACGTACCTCGCAGGCCGTGGGTATCCGCAGGGACAGATAC
ACTCTGTGTATCCTTAAAACAAGTGCAATTGAATGTCCATTGGCCTAAAGGGCTTAGAGCCTTCATAGCA
GGGCCTTCCTCAGTGTATTCAATTCGGGTTAATTTTGCTGTCTGATTTGCAAGGATTGGTGTTGTCATCT
TTTACCTTGGCTTTGTCCAAGTTCCAGGATTTCTTAGAAATGCCGTTGACGAATTCACGGGCAGAGCCAG
AATGTCTGTCTTTAGGCATTCTTGTCTGCACAGAAAAATAGGTAGGTTAAACATGCAACTTTTTATCTAA
TGTGGTTTTGTTCTTGTTAATGGTTCCTTTATTTTGACAGTTCAGAAATGTCCAGAAATGACAAAGAACC
GTTTTTTGTGAAGTTTTTAAAGTCTTCAGACAATTCCAAATGTTTTTTTAAAGCTCTCGAGGTAAGAGTT
GAAAACATCTTAATACCGTGAATTCATTTTCTTGTTTTTAAGTGTTTTTGATTTGTTGGGCTTTATGTCT
TAGAAGTAGATATTTCACTTTCATAATAATTTTATAGTCTACATTAATACAGATATATCATTATCACATA
TTTTTGAATTGATCTAAGTACATCATTATGTATTTCGGGGGGCTGGAAGTCTATTATCACATTGCTGTTT
AATATGGCTGACCTACAGTTCTTGGAAATTAAGTTGCTTTCATTTGTCTCCTAGCCAGAGTGGTCCTTTG
CGTGCTGTAGAAATTTGAGTCAGTTGTGTACTGGTCATACTTGAAAATTTTTTGGTTAATTGTTAAAAAT
TATGTTTTATTATGTACAGTATAATAATTGGATGATAGTAGGGGTGATGTAGATCCTTGCTTCTCACAGA
ACCCAGCAGTATCAGCATGGCCTGGAAGTTTCTTCGAAATGCAGAATCTTGGCCTCCAACCCAGACTTAG
AATCTGCATTTCTAACAAGATCTTCAGTGTGCATTAAAGTTTGAGAAGCATTTGTAGCAGATTTTTTTAA
ACTTCATTCATTCAGCAAGTATTTATTGAAGTGTTCATGATTTGATAGGCACTTCTCTAGTACTTGGGAT
TTATCAGTGATGAGAACAGGCAAAGATTTCTGTTCTTTCAGAGCTTACATTCTAGCAGGAGGACATAGAC
ACTAATCAGACATGATAAATTAGGAAGAGAAAAAAGCAAAACACGTTAAGGGGTTCCAGAGGATGTTTCT
TGGGGTGGGAAGAGAAGGGGGTTATAATCTTAAATAGGGTGTGATCAGGGTAGGCTTTATGAAGAAAAAG
ATATTGAGTAAACGTGAAGGAGGTGAGATAGCCTTGCAGATATCTGGAGAAGAATGTTGCAGGCAGAGAG
AGCAACCTTATACAGCCCCTGGGAAAGTGCCTGTAGTCTCTATCCAAGTTTAGTGTACTGTTGTGTAGTG
AGTGAGGGATGGGGGAAGAGTACTAAGGAATGCGTTAGAGAATAATGGTAAAGAGGTCATGTAGGGCCTT
GTGGGCCATTTTTCTCTCAGTGGAGAGCCATTGGAGGGTTGATAATCAGAAGAGTAAACATGATCTGACT
TACAGTTTTAAAGGGATTACTGTATTTAACATAAGGACCTCAAAATTGGTTCAGAACATTTCTGAGTGCT
TACTGGGTGTCAGGATATAGTAGCAAACAAGATACATAGAAACTTCCAGGGGAGAGAGACAAAAACTATA
TATATATATATAAATACGTATTTTTAAATAGAATAGACTAGAAGGGTAGAGGAAGGGTATTTGAGAAAGC
CTGATTAGGATGGGCATCTCTGGGGAGGTGACATTTAAGCAGACCTGATTGAAGTGGGGGATTAAGTCAG
GTGAAAACCTGGAAAAAAAGTATTCTGTGCTGCAAGATCAAGAGCAGAGCCTCTAAATGAGGTTATGGTT
GGCTTGTTCCAGGGTTGGCAAGAAAGCGGGAGTGGCTGGAATAGAATAAGCAAGAGAGATGAGAAGTAGG
AGGTAAAGGTGGAAAGATAGGCAGGAAACCAGACTGTGTTGTCTTGAAGGCTGTAGTAAGAAATGTGGAT
TTTATTCTGGGTGTGGTGTGAAGACATTAGGTTTTGAGCAGATAGTGACATGATCTGACTTGTGTTTTTG
AAAAGATTATTAGCTGCTGTGTAGAGAACAGATTTAGGGCAAGGAAGGGAAAAAGAGACCACTTGGGAGC
CTTCTGCAGAGGTCTAAGTGAGATGTGATGGTGGCTTTCAATAGGGAAAAGGCTGGATTTGAGATGGAAT
TTGAATAGGACCTAAATACAGGAGAGTAATTTCAGGTACATTCTTATTTTATTTTTTCTTTCAGTCCATA
AAAGAATTCCAATCAGAAGAATATCTTCAGATTATTACAGAAGAAGAGGCATTGAAGATAAAGGAGAATG
ATAGATCACTTTATATCTGTGACCCTTTTAGTGGCGTTGTCTTTGATCACCTCAAAAAGGTTTGCTGACT
TCTAGTGTGTACCTTTGGTAGTCTATTTTTTGGTTTATCTTTGCTTTAATGGACATACATTCTTTGCGGA
TTATAAAGTACTATGCAACTTATGAGTACATTGGAATTCTTTTGGTTTGGCAAGACCAGGATTGAAAGGC
TAATTATGTTACAGTAGGCTTTTTTTGTTTGTTTGTTTGTTTTTGTTTTTGTTTTTTGGAGACAGAGTCT
TGTTCTGTTGCCCAGGCTTTAGTGCAGTGGCACAAGCTCAGCCCACTGCAACTTCTGCTTCCTTGGTTCA
AGTGATTCTTGTGCCTTAGCCTCCTGAGTAGCTGGGATTACAGATGTATATACACACCCGGCTGATTTTT
TTATTTTTAGTAGAGAAAGGGTTTCTCGATGTTGGCCAGGCTGGGCTGGTCTTGAACTCCTGCACTCAAG
TGATCCTCCCACCTCAGCCTCCCAAAATATTGGGCTTATAGATGTAAGCAGTAAGGCAGTTTTCAGTGAA
AGGGGCTTTTGAAGTTTATTTCTTTTCTTTTCTTTTTTTTTTTCTTTTGTTATAGGGTCTCACTCTGTCG
CCCAGGCTGGAGTGCAGTGACGTGATCTCAGCTCACTGCAACCTCCGCCTCCTGGGTTCAAGCAATTCTC
TTGCCTCAGCCTCCCTAGAGACTGGGACTGCAGGTGCCCGCCACTGCACCCGACTAATTTTTGTATTTTT
AGTAGAGACGGGGTTTCACCATGTTGGCCAGGCTGGTCTCGAACTCCTGACCTCAGGTGATCCACCCCCG
CTCGGCCTCCTCTGAAGTGCTGGCATTACAGGCGTGAGCCACTGTGCCTGGCCTGAAGTTTATTTCTTTT
GATAGCTTGCATTGTTTGATTTGCTTTTGTGTGTGAGTGTTTAATGTATAGTATCTCTGAGAAATTATAT
AGTCATCTTTATTTTTTTCTTGTAATGATTTGCTGATTTCTCTACAGTGGTCATCAACTTTGGTAAAGAA
TTGGTGAATGTTACAAAATGTAATTTTGTAAGACTGGTGGTCTTTTCCTTCTTAACTCATTTAAACTTTT
CTCTTTTTTGTAGCTTGGCTGCAGAATTGTTGGTCCTCAAGTAGTCATATTTTGTATGCACCACCAGCGA
TGTGTCCCAAGAGCCGAACATCCAGTTTATAATATGGTTATGTCTGATGTAACCATATCTTGTACAAGTC
TGGAAAAAGAAAAAAGGGTAGATATTGATCTCATGATGATTGATCAATTTATATATCTATCTCTTATTCT
TAATTTTTCACTGCATAGAAGATAGCTTAAAAGACTTTCCGCTTATTGTTCGTGACTTTTAACAAAGTTC
AGTATACAACCACAGTGTGTATATATGTATACATGTACATTTTTTAAGTAAATTGAATATAGTAGAATTT
GTTTTGTTGAAAACTGGCTAATTGAAATTTTTTGTTTTAATTTAAAAGGACACTCATTTTTTAATCACTT
ACATTTTTCCACTATAGGTGGACAGTCTTTAAAAAAAAGGATTCTAAATCGTTTGAATCAGGGAGGTGGA
GGTTGCAGTGAGCCAACATCACGCCACTGCACTGCAGCCTGGGTGACAGAGCAAGATTTCGTTTTGGAAA
AAAAAAAAAAAAAGCATTCTAAGAGAGAGCTATAGAAATACATATATATATTTGGGACAGGGTCTTACTG
TCACCCACGCTGGAGTGCAGTGGCACAATCACAACTCACTGCAACCTTGATTTCCTGGGCTCAAGTAATC
TTCATGCCTCAGCCTCCCGAGTAGCTGGAATTACAGGCATGCACCACCAAGCCTGGCTAAAAATAAATAT
TTATATATTATATAGATATAATTTATGTTGTTAAAAATACACATGTATATTTATATAGAAGCAAAGATTA
AGATTTATACATCCTTTGTATAATAGTTTAATACATTCATGGAGTTGAAAGGGATAAATATAGGAAAATA
ATATTTTAAAAATATAATTCATACTTATCTCCCAAAAAATAATTTGGTTAATTATTTAACTTTATAATGT
TATGTAACACTTATGTATTCAAACTGTCAACAGCAAAATGTATTTATGAGATAGTGTATAAAAGTGGAAG
CAATATTGTTTGCTTCAGCATTAATGTGTTCTCTTTTGGGGCTGCAGGAAGAAGTTCATAAATATGTACA
AATGATGGGCGGACGAGTATACAGAGACCTTAATGTATCAGTAACTCACCTTATTGCAGGAGAAGTTGGT
AGCAAAAAATATTTAGTTGCTGCAAACCTGAAGAAACCTATTTTGCTTCCCTCTTGGATAAAAACACTTT
GGGAGAAGTCACAAGAGAAGTAAGAAAGACATTTTTATATTTTCTAGATTTGAAAAAATGATGCATTCAA
TCATTTTTGGCATGTGATATACTCTTTGGACTTACTATTCAGGGAAAAAAATAGTGTCATTACTCTTTTC
TCCAGCGAAAACGGAAGAGCAGAATCTACAAGCTCTTATTACCAAGATATAAGTTGACAGGCATAGAATG
GGCCAAGGGTTTAGCATTAGCTACAGAGTAGGAAATGCTTTGGTGATAAAGTTCTCAGCTGTAAATATTT
CTGAAATAATTGAGAAAACGAGTTTGGCAATTCTGTAGCTTTTATGTTGCATTATCCTTCAACACCATTC
CCATATGTTAAGATAGATAATAAATTAGTCATTTTGGCAGTTGCATTCCTTCCAAAAGATGGCATATGTA
AGGTTGAATATAAACATATGCAAGTCATCAGCTGAGATGTGATCATTTAATAAATGCTAATTGAGCCCAT
TTTACGTATCTGACTTGAGGTAGGCATTAGGTATATGTAGTAAGCAAAACAAGATAGTCACTTCCCTCAT
GGAGCTTAGAGCCTATTTCAAGGGGCACACTTCAGTTTCCCCTGCAACTAGATAAACAACCACAAGACCA
AACACCACAGAATTGTATTGGCATACGAATTATATCGGCACTTTTCTGGGCTTGTTTTCAACACCATCCA
GCATATCCCAAATATGTCTCCATGTCAGCAACTCTCATTCCCAATGTGATTACTTCATCAGCACACCTCC
CTAGCATGGCAACAGAATAATGATATTTTTGTTGGTATTTTGCCAAAAATCATTGCTTTTACTTGTATAG
AATAATGGAAAGATTAAAAAGTTCATTTATCTAATAGGCAACCAAATTACCTTTGGGTTTTTAAGTAGAG
ATTCAGACTTATCATTTTGGAAAATATACTATGACCAGCAAGGGCTAGAGTTAAGGAAATATATATTTTT
CCCTTAAAATGGTTTTGAGAGACTGTATCTGAAACAATTACTTTGTTTTAGAGACATGTTATTGAACTAG
GTGAACAATTAGTGTAATTCTGTTTTGGTGGTTCTCCTTTGTAAAAAAAAGATTATAAATCCTAAAAAAT
GAGGGAAGCAATAGTGATATTTGTGTTGTTTTTCATTATTCTTCTGGTAAATTTAATATGTTCTTTTTTA
ACCATTTGATTCCCACAGAAAAATAACTAGATATACTGATATAAACATGGAAGATTTCAAGTGTCCTATT
TTTCTTGGTTGCATAATCTGTGTGACTGGCTTATGTGGCTTAGACAGGAAAGAAGTTCAGCAACTCACAG
TTAAGCATGGAGGTCAATACATGGGACAATTGAAAATGAATGAATGTACACACCTCATTGTGCAAGAACC
AAAAGGTAAAACTGTGTTTCACAAAGGGACAAATGAAACATTTTCTCTAGTATTTAATATTTTATTTTTA
TTTATTTATTTATTTATTTTGAGACAGAGTCTTGCTCTATTGCCCAGGCTGGAGGGCAGTGGCACAATCT
CAGCTCACTGCAACCTCTGCCTCCCTTGATCAAGCGATTCTCCCAACTCAGCTTCCCGAGTAGCGGGATT
ACAGGCACCTGCCACCACGCCTGAATAACTTTTTATTTTTAGTAGAGACGGGCTTTCACCATGTTGGCCA
GACTGGTCTCTAACTGACCTCAATTGATCTTCCCACCTCAACCTCCCAAAGTCCTGGGATTACAGGCATG
AGCCACCGTGCCCAGCCACTAATATTTAATATTTTAAAATCTTTACAGAACAGCTTTTACTTCTGACATG
TCATTAATCTAGTTTGATACTGTTTTCTCTAACATCTTGCAAAGATGTTTTCTCTAACATCTTGCAAAGT
TTTAACTGGAAGACACACCTTGCGGCACATGAGGCAGTAAAAAATGAAAAAGACATTACGAACAAGAAAA
ATTCTTTAAAATTGCTATTAAGTTTGGTGTTACATAACATTATTCTGGATAATTTTTTATCAGCACTCTT
AAAATATACACAAATTGATAAAAGATGAGTTTTGTTAATGTTTTTATTATTGCCCTTTATATGTATACTG
TGTTTTACTAAGCTATCTAGATTGAGAAGAAATTACGATTAAATGGTAATTGTCCTTAAGTCAGAACTTT
GAGTATGGCTGATTGTGATAGGATCACTGTGAGTAGAGTTGATACTGCTGTTCTTTTTCTGAATCTGAAG
ACAGTTCATTGTTGATTCTCTCAAGAATGCCAAAAAACCTTTCATGTGACATATTGACCTCAATTCCTCT
GTGATACTGCTTCAAAATACATTGTGTTGAAGATTTTTAAGGTAATAAAAGATACATATTTTCAATATAA
TTTATCTTTTGGCTTATCTATCCTGGACTTTAAGTAGAAGCCTGTTGACATTGGAATAGTCTGTTCAAAG
TTGGGATTTATTATATAGCACAAATTTACAATTTTCACATTGTACAAGCTGGTTTAAAGAAATTAAGTGA
TGGAGCCTATATGCAAACCCAAGTCCAATATTAATTAAGTTACCCCACAGTTGCTTCTGTCTTCCTGCAG
TTTGTATAAAAGATAAAACCTCTCACATGGCATATGTATTTATACTACACATAGAAAATATAATTAGGCA
ATAAGCTAAAATACTACTTTGTAATAAACTGTGTAGCATACTGTTTAAGAGTAAGTACTGGTAACTTTAA
TTTTGTTGATAGGTTTTTGAAATAACCTGTTATAGCCGGGCGCGGTTGCTCACGCTTGTAATCCCAGCAT
TTTGGGAGGCCAAGGCAGGCGAATCACCTGAGGTCAGGAGTTTGAGACCAGCCTAGCCAACATGGTGAAA
CCCCGTGTCTACTAAAAATACAAAAAAATTAGCCAGACATGGTGGCACACGTCTGTAATCCTAGCTATTC
AGGAGGCTGAGGCAGGAGAATCGCTTGATCAGGAGGGAGGTGGAGGTTGCAGTGAGCCAAGATCTCACCA
CTGTACTCCAGCCTGGGCAACAGAGTGAGACTCCATCTCAAAAAAAAAAAAAAAATATTAACCTGTTATA
ATTTGAATATTTCTGCAAAGATAGATTGTAATGGTTTTCATTTCTTGGTTTCTCCTATTTTCTCTATTTC
TTTCTTAATGAACGCCACAGGTCAGAAGTATGAGTGTGCCAAGAGATGGAATGTACACTGTGTGACCACA
CAGTGGTTTTTTGACAGTATTGAGAAAGGTTTTTGTCAGGATGAATCCATATACAAGACAGAACCTAGAC
CAGAAGCAAAGACTATGCCCAATTCTTCAACTCCTACCAGCCAGATCAACACAATTGATAGTGAGTCTCA
TTGAGATTAAAATAATTATTCTGAAGACAGTCATATTTCTATGTAATAATCAGTATGTTAATAGGATCAC
TGAGATAGGGAATTTCTTTTTCTTGTTTGCTGTGCTTCTAGGCATAATTGTATAATAGTCAAATTGTATA
AAAAGTTAGCACTGAACTGTTCGTAATTTTCTTTTAGAATGAATGAATGCTGTCTTTTATACTCTGTTGT
AATGCAAAAGATGATCAAAGAAATTAAGAACAATGGTAGCTGGGGGCTTAAGATAAAAGTATTAATCTTT
TAAACATTACATGCATATATTAATCTTTCCTTAATTTACAGTTTTGCTGCAGTTATAGATCATCTGTATT
TGTATAAAAATTAAAACCATTTCTATTTTGTAGGTGTCGTGTTGGCAAAATGTTTAAAATATTTTTTCAA
ATGTAAGCTTCTTGGACCTTAAAGTCAGCAACTTCAGATTTTTATGGGAAACAAGACACTATTAAGAAAA
TGTGTTAAATCTATTTTCATTTAAATAAGATAGGAGGCTTAATTAATTTCATTTTCCCTTTTTATACTTT
GCCACTAAGATTTCAGTAAACACCCCTGAATCTTACTTTGGAAGTTGTTTATTGCCCTATATTTGTAAGA
AAAGTTAATCCATAGACAATATGTAATCATGATTATTTTATCTCCCATAAATTTATACGTTTTCAAAATA
TGTAGGTCGTACTCTTTCAGATGTCAGCAATATTTCCAACATAAATGCAAGTTGCGTAAGTGAATCAATA
TGTAATTCACTTAACAGCAAACTGGAGCCTACACTTGAAAATCTAGAAAATCTGGATGTCAGTGCATTTC
AAGCACCTGAAGATTTATTAGATGGTTGTCGGGTACGTCTTTATTATATAATACATGCAGTAGATGATAT
ACATTAGCATTTCCTAGCCTGACTTTGAAGACCCTTCAGAGTTGGGCTTGCTCCTTACTATCTATGTAAA
CCCTCCACTCTAGCTAAGTAGATGCACTTACTAGACCATGTCCATCCTTTGGCTGCGAGAGTCCCTCTGC
CTGCAAGGTATCTTCCTCTGTTATCTTTTAAAAGTTCATTTCAGGTTTTAACTCTTCTCTGAACCCTCCT
AACTACATTAATCTTTTCATCTGCTATATTTCGAGAGCACTTAGTTTCTGAACAACTTATTGGATTTTTT
TTTTTTAATTTTTTTGAGTTAGGGTCTCACTCTGTTGCCCAGGCTGGGATGCAGTGGCACAATCATAACT
TCCTGTAACCTTGAACTCCTGGGCATCAGCCACCCCTCAGTCTCCCGAGCAGTTAGAACTACAGGCATGT
GCCACTACACCTGGCTAATTTTTAAAATTTTTTTGTAGAGACAAGGTCTCACTATGTTGCCCAGGTTTGG
ACTTTAAACTATCATCCAAGTGTTTACAGTGTATTAGACACTGTGCTAATCATTTTATGTGTGTTAATAT
ATATATAAAATCGCATTTAAGTCCCACAAATAACTCTAAAATGGATATTATCTTGAGGGTAAGACTTAGA
GAATTAAAATTCTTGTTCAGAGTCACACAGAGAGACATTAAGTGGCCAGTTATCTACTCCAAAGTCTGTA
TTCCTCACCATTAGAGCCACTGCTTTGTTTGGTATTTCTTGGGTTATTTAGTTTTTTAGTATATGAATAA
CTGTCTTATAAGTTTGTTGATGGAAACAGCATATGCTATACTTTTTCCGCTGTCTTAGATTGGAATACTT
AATGTATTCCTATTTGTAGAATGACTTGGCTGTGCACTAATTTTCACAGAGTAGCAAAAATTTAACCCTA
CCTTATTGTTAAGAAGGGAAATTGGAGCTGGGCTTGGTGGCTCATGCCTGTAATCCCAGCACTTTTGGGA
GGCCAAGGTGGGCAGATACCTGAGGTCAGGAGTTGGAGACCAGCCTGGCCAACATGGTGAAACCCTGTCT
CTACTAAAAATACAAAAAAAATTAGCTGGGCGTTGTGGCAGGCCCCTGTAATCCCAGCTGCTTGGGAGGC
TGAGGTAGGAGAATCACTTGAACCCAGGAGGCGGAGGTTACAGTGAGCCGAGATTGCGCCATTGCACTCC
GGCCTGGTCAACAAGAGTGAAACTCCATCTCAAAATTTAAAAAAAAAAAAAAAAAAAAAAAAAGGGAAAT
TCGGCAAATTACCTTTTTTTTTTTTAAACTTGCCAACTTACAAAAGGTGGGTTTTTTGTTTTTGTTTTGA
GACGGAGACTTGCTCTGTCGCCCAGGCTGGAGTGCAGTGGCGTGATCTCAGCTCACTGCAACATCTTCCT
CTTAGGTTCAAGCGATTCTCCTGCCTCAGCCTCCTAAGTAGTTGGGATTACAGGTGCATGCTAATTTTTC
TATTTTTAGTAGAGGTGAGGGTTTCACCATATTGGCCAGGCTGGTCTTGAACTTCTGACCTCAAGCAGTC
TGCCCACCTTGGCCTCCCAGAGTGTTGGGATTACAGGCATGAGCCACCACACCTGGCTCAGAGGTGTTTT
GTATGATGTTTTATCCTAGACTACTAATTCTAGATATAGCTTTTAATTATTTCTTACATATAGAAAGTAT
ATGATTCCAAATATTTGTTAAAATATATTCTAGTAATGGGGGTGATTTAATTTGGGGTATAAATGCAGTT
TGGAGTAGATTTGTCATACTTAAATTTTTTTAAGGGCACCTTATGTAAAATAGAGGTAAAATGATTTTAT
ATTGATTAAATTGGTTTAAATATATATATACTGATAGCTAAAAATAAATTACTGCATACATGTTATCCTC
AAAGCATTGTGTTCAGTTCTTTGACTTATATATATTTTTTGTTCAGTCAACTCTGTGAGTAAGGTAACAT
CAATTTATAGGTAAGAAAATTGATTTAAAGAGCGTAAGCAACTTGCCTATTGTCGTTAATCATAGGCAGC
TGGAAAAAACAACCCCTCCCCCCGCCCCCCACACACACACAATTTAAGCCCAGGTGTTCAGAATTCAGAC
AGTTAACTACAGTTTACTTTTATAATTACTTTAAAATTTACAAGTAAGCTTCACAGTTAAAGAAGTTACC
TGCATTTTAGGAATTGGTATGCACTAATATTTAGAAGATGTTGGACTCTGGCTTCTATTTTCTTTATAGC
CACAAAATACTTATCAAGTATCTACTGTGAAAGATAATGCTGTATGAGATTGTAAAGAGTGATTAAAAAC
TCAAAGTTGCAGAACTGTTAAAAAATCAAAAGTTTCAGAATGTATTTATTCATATATCTTAAATGTACAC
AAGTTGTCTTATTTATACACAGTGGATTTAGATATATATTTTTAGTTTTTTTCTATTGAGAGAAAGCATG
TTATGTATATTAAATATTTTCTTTCTTGCAGATATATCTTTGCGGTTTTAGTGGCAGAAAGCTAGATAAA
CTGAGAAGACTTATTAACAGTGGAGGTGGAGTTCGTTTTAACCAGCTAAATGAAGATGTAACTCATGTTA
TTGTGGGAGATTATGATGATGAATTGAAGCAGTTTTGGAATAAATCAGCCCACAGGTTTTTTTCGTTTTT
AATTCAAAGCAATTTCTACTACATAATTTTTCTTAAAATTGCATATCCACGCAGTTCTGGAAACTGCATG
ACAAGTGGGATGATGGTCTTTATAGCTTGCACTAATACATAACTATGTGCTATTTTTGTAGGCCTCATGT
AGTGGGAGCAAAGTGGTTGCTAGAGTGTTTCAGTAAAGGTTATATGCTTTCTGAAGAACCATATATCCAT
GCTAATTACCAGCCAGTGGAAATTCCAGTTTCACATAAGCCTGAAAGTAAAGCAGCTCTTTTAAAAAAGA
AGAACAGCAGCTTCTCTAAGAAAGACTTTGCTCCTAGTGAAAAGCATGAGCAAGCTGATGAAGATCTGCT
CTCTCAATATGAAAATGGTAGCTCCACAGTAGGTAAGAAATGCTTGATTAGTATTTGTAGTTAGTAAGAA
ATATTTTAGTAGCCTAATACGTACATATGTGATTTCTGCCAAGTTTTCTAGATTTTAAGTTAAATTAACC
TTTTTCTTTCCTTAAAATTTTCCTCATAACTTTTTTTCTGAGCAGGCAAGACAAGAGGGTCCTGAACCCT
TTAACCCTCTCTCCTGTCTCCAGCTAACTAGCTCTATTTTGATTTGTTATATATACAGGCATTGCTTGGT
TTGTGTGTGAGTTTGTAATATCATGAGCCTTTTATTAAAACCTTGTAACAAATACCTCTTACTGGATTAT
TGTGGTCTTTAATGCATGAAACTGTACTGTGGACTACCACCTCAGTATGTACTCTTATCATGTAGTAAGG
TAAATTGCCAGTGAAGCATTATAAACATCATTACTTCTTTTTTATTAAAAAAAAGCCTTTATAAAATACC
TAATATAGCCAAAAATATATACATTTAAGTAAATTTAATGTGTATCATAACTTAAAATCTGAAGTTAATC
AGAATTTTAATCTACCTCGAATAAAACAAGAACCTTAGAAAACTTTTTTTTTTTTTGAAGCAGGATTTCG
CTCTTGTTGCGCAGGCTGGAGTGTAGTGGCATGATCTCAGCTCACTGCAACCTCTGCCTCCCAGGTTCAA
GTGATTCTTTCTGCCTCAGCCTCCCAAGTAGCTGGGATTACAGGCATGCGCCACCACCCCTGGCTAATTT
TGTATTTTTAGTAGAGATGGGGTTTCTCAGTGTTGGCCAGGCTGGTCTTGAACTCCCGACCTCAGGTGAT
CCACCCGCCTTGGCCTCCCAAAGTGCTGGGATTACGGGCGTGAGCCACTGAGCCCAGCCTAAAATACTTT
AATGATCACAACTAGCCCTGCTCCCTCTCTCCACTCATCAACATACTCAGCTTATGTGATTTTGTCTAGT
ATTTTCATTATCTTTTAACATCTTACAATTAGGCGTTATTATGCTTTTATATGGTCAGTATTTGTTTCAT
TTACTAACACGTTTAACTGTTTCTTTGCTCTCCCCAGTTCTTTTTACATCTGAGACCTCTCTTTTGGAAT
CATTTTCTTTCTTCCTAAAACAATATCCTTTAGAAACTCCTTTAGTTTCTCTAAGACTCTTCGGCTGGGT
ATACAGTTCTAGACAGTTTCTTTAAGCACAGAGATATTGTTTCATTGTCATCTATTTCCCTGACTGCTGG
ATAGGAGGTCAGATATCAGTCTGTCATTCCTTTACAGATAATCTGTCTTTATCCTCTGGCTGTTTTTAAG
ATAGTCTTCCTTTTGGTTTTTGTGTCCTAGAGCTTTACCATTTATTTGTCCTGCTTGAGATTCACTGTGC
CTTCTGAATCTGAGAATTAGTAGATTTTTTGCTTTTTGTTAGGTCAAGAAATGTTTATTATCTCTTTGAA
TATTGCCTCTCCCCCATTCTCTATATTCTCTTAACTCCCATTTGCTTTGCATATAGGTCACTTAACCTGT
CCTTATCTTCCATCTTTTTGTCTTTCTGTGATTTATTATGTGTAATTCCTTCAGATTTTTTTCCAGATCA
CGAATCATCTCTTCCGCTATTGCCTAATCTGTTTTACCCATCCATTAAATTTTCTGTTTTAATAATTAAT
TTTTCAATTCCTAGAAGTTTTAGCTTTTTAAATAGTCTGTCATTTATTTTTTTACTTTTAAAAATAGAGA
TGGGGTGCTACTATGTTACCCAGGCTGGTCTTGAAGCACCTCAGCCTCCCAAAGTGCTGGGATTGCAGGT
GTGAGCCACCACACCCAGCCAAGTCTGTCATTTTGTATTTGTATTTTTTGTTCTTTATTTCTTAAAACAC
ATTAAACCTATCAAAACATCTGTGAATTGTTCTGTTGGCTGTTGTTTATGGTGCCTGGTTTTCTTGTGTT
TTGTGATTTTTATTGTGAACTTGTACTTACTAGGACTCTAGGAATTCTAAGAAACTTGGGGTAGGGGTTT
GTTACCCCAGAAAAGGTTCATGTTTGGGTCTGCTAGGTGCCTAGGAGTTTACAAACTCAATATGCCTTTA
AGTTGGTGTATTGGCTGGAGGTTTCTTAGACCTCCAGCTATATGAATTTATACCCAAACCTACTTTGAGG
ACCAGCCTCTGATGAACAGATTCTTAGGAGAGATGTTGTTTCCCCTTGACACAAAGCCAGTGTTCTTGAC
CTCTTCCTTTACAGGGCAGATTTGTTGTTGGTTGGTTGGTTAGTTTTGGTCTGTTTACTAAAGATATGTC
TCTTCATGGGTTCTGGGCTTGTTCTCTCAGGGTATACTCTCCTTCACCTCTAAGTTTTATGAAACCAAAG
GATTGGCTCCTATCTCTTGTGTGATACCTTTACATTCCAAGGGTCTGGGTTACTGCACAGATATTTAATA
GATTTTGACTAACCATTAGCTTTAGTAACTACTTAATGTTCTGGATTTAGTAACCACTTTAGTGGTCTGG
ATTTTAATATTTTATCCTGTATTTTGAAGTGTTTTTAGTGAGAGTGTTTCAAAGATCCTAGCCCACCATA
TTTCTAGAGAGGGAATTCCTAGCATTCCTTTTTTATTTACACTATTTCCGTACTACAAAATTGACTTTTC
TCAAACCATTTACTTATGCTTTAGTATTGTATCACATCAGGAAATCATCTTGATTTTCCAGGATGCTGTG
GTATAACTGAAAGTGATTTTTTTTTTTTTTTTTGGAGACAGGGTCTCACTCCATCACTGAGGATGGAGTG
TAGTGTCACAAGCTTGGATCACTGCAACCTCCTCCTCCCGAGTTCACGCAATTCTCTTCCCTCAGCCTCC
TTAGTAGTTGGGAGTACAGGTGTGCACCACCACGCCCAGCTAATTTTTGTATTTTTAGTAGAGATGGGGT
TTCACCATGTTGGCCAGGTTGGTCTCAAACTCCTGGCCTCATGTGATCCTCCTGTCTTAGCCTCCCAGAA
TGTTGGGATTACAGGCTTGAGCCACTGCACCCAGCCAGCAATTTTACATACTATCCTTCAGCCAAGGGTA
CTTGGTGTAGGGCAGGTGATTAGTTTCTTAAACCTTCCTAAGACAGCTAGTGGTTTACTGACTTTATAAG
GATTAAGTGATGGTAAATCTTAGTGGATCACCAAGCATCTTAGAAAGTCTATAATAAAGTATTTTATAAT
TAATTCTTAAAATGGCTTTTGATTTAAGGGAGTTTTTTGTTTTTTTTTTGAGATGCAGTCTTGCTCTGTC
ACCAGGCTGGAGTGCAGTGGCGTGGTCTTGGCTCACTGCAACCTCTGCCTTCTGGGTTCAAGTGATTCTC
CTGCCTCAGCCTCCCAAGTAGCTGGGATTACAGGCACGTGCCACCATGCCCAGCTAATTTTTCTATTTTT
AGTAGAGATGGGGTTTCTCCATGTTGGCCAGGCTGCTCTTGAACTCCTGACCTTGTGATCCACCCACCTC
AGCCTCCCAAAGTGCTGGGATTACAGGCATGAGCCACCGCGCCCGGCCCTAGGGGAGCTTTTTGAATGAT
AGGTTTATTATGTAGTATCTCTTAGGCTTTTGGGTGCGGTCTGTTTGAAAAGAGATCCTACTGCTAAAAT
AAACGGAAAGAACTGCTGTAGAACATGTTAGTTTATTATGAGTCTCTTAATAACTTCAGAGTTTTACTGA
TGGTTTATAAGAACTTAGTATTTCTACTTTGTTTTTATAGTAGTAGTCTAAAATTCTACTGGATTTCTTC
CTCCTTCCCATCTTACCAACAACATATAGACGTCGAAAAAAGTTATTTTTTTCTCCTAGATGTGACTGCA
AAATAATTGTACTTGCAAAAAATAAGAAGGTTTTTACTTATAAAACAAACTTTACATTTTAACAAAACTG
TTTAAAAAACTTTCCGGAATGTTTTATAGTATTGCCTCACCTTTAATTCCATCTAAGCATATTCATTCCC
ATGAGTATATCTGTAAATTATAAGTATACTATTACCCTTTATTCCTTATATTATGAAACCAAGGGTCAAA
GAGACTATAAAACTAGAACCCAGCTCTCCTGACTTACTGACCAGTAAACTCCCTAGACAAAACTATGCTT
CCTTAGTGCGAATAAGTACAATTTTAATACTTATGTTTAGTAGGAGTCATAAGTTCCTAATATATATTGC
AGTGTACTTAATTTACAAGTTCCTAATGTATATTACAATTTACTTAAAAATGAAAATGCTAGTGTCTGTT
GGAGATGAGGATAGAAGTACGAAGATGGCACTATAACATGGTGGTGTGAACTCTGGAGTTAGACAGACTT
TGGTTTGTATCTCATTTCTGCCACTTAACTGTGTGACCTTAGGCAAGTTACATAACCTCTTCAAGCCTGA
GACTCCTTTTGTACAAAAGGGATGTACTGATAGTATCAGCCTCACAGTTAGCATTATGTGAGCATTACAT
GCATTTGTAAAGCACTTAGTCACAGTGCTTATATAAATGAAAGAGTGATTCTTGGATAGCTTCCAGCCTT
GTTGGAATCACATAGCAGGGCAAACTTTTTCTCTAAAGGAGTAGACAATAAATATTTTAGGATTTGCCAG
CCACTTCCTTATAGTTATTTCCATGTTAACATGTTCCATGTTAAGGACTTGACAGTTATACCATTTAGAC
TTGTTAATTTTTTTCTTAATGATAGTTTCTTAATTTTATGGTGAATCATCTAAACCAGTTTTGGAACTAA
GTCCTGTTGGAGCTATATAATTTTAAACTAATTCAAGTTTCCATTATACTAGGAATACCTAGAACTAACA
TGATTGCTCTTGTCCTTCCCAATGGATGATGGTAATACTGATAAGGACTCTTGGTAACCCCGAAGACGGG
GTGAATCCATATCCCTTTATTATACACGTAAGCTTTAGTTTTGTAAGTATGGTCAAAATTATCAGATTCC
ATGTTACGTTAATATCCTTGCTAGGAAGTAAACTGTATAAGGTAAATTGTTGGTTAGAAAACATTTACTT
TTCTCTTTCAGTTGAGGCTAAGACGTCTGAAGCCAGGCCCTTTAATGATTCTACTCATGCTGAGCCCTTG
AATGATTCTACTCACATTTCTTTGCAAGAAGAAAACCAGTCTTCTGTCAGTCATTGTGTCCCTGATGTTT
CTACAATTACTGAAGAAGGCTTATTTAGCCAAAAGAGTTTCCTTGTTTTGGGTTTTAGTAATGAAAATGA
ATCTAACATCGCAAACATCATAAAAGAAAATGCTGGGAAAATCATGTCCCTTCTGAGCAGAACTGTTGCG
GATTATGCTGTGGTTCCTCTGCTGGGGTGTGAAGTGGAAGCCACTGTGGGAGAAGTTGTTACAAATACAT
GGCTGGTAAGACAACACTAGTGGTTGAAATAAAGTATCGAAGGATATCTGAATGCCAAGAAGGTTAATGT
TTATTTCTTAGACAGTTCAGTTACAGTAAATCAATGCTTGGACTTGAACACTCTTGATTTTGCTTTTAGC
AAGGGAAATTCAAAATTAAATGTGCAAGCTTCTTGGCCTAATCCCGATACAGTAGAGATTACTGTTTCCC
AGGGCCACAGCAGCAACATTCCTTCTTAGGAGGTCACTCTAGGGTCATCTCATACTATTTGTCTTGTTTT
CTCATAACCAAAGCATCACTGGTAATTTCACTGTTGATTTTTTTTTTTTTAACATTCCAAGAATTATGAG
AAAAATAGTATAAACTAATACATTAGAAGGAATGAAATTTAAAGCAACATTCAGTAACTCTAATAATAGA
TCTTACCGATTTTAATAGCCTAATGTAATTAGTCGATGTATGTATATGTGTGTATGTATATATATACTCT
AACATTATGATATGAAATGTATTGAGAATGAGCATACACAAGTTTGTATTAAAGTAAAATTTCTTCTTAT
CAAAACTAAATTATTGCTTCTGTACCTGAACCAGTGTTAACCACTGGTTGCTTATTTCAGGTTGGCTTTA
CTATTTCTTTTGTTTTTAAAAGGTTACTTGCATAGACTATCAGACTTTGTTTGATCCAAAGTCGAATCCT
CTCTTCACACCAGTTCCAGTAATGACAGGAATGACTCCTTTAGAGGATTGTGTTATTTCATTTAGCCAGT
GTGCTGGAGCAGAAAAAGAGTCTTTAACATTCCTAGCAAACCTCCTTGGAGCAAGGTATGTAATATTTTT
AATGCACCTGTTGTATTGGTGTATCTTTTTATTTATTTTGACTTCTTGGAGAGCATTCAAATTAGAATTT
GGTTGGGTGTCTTTATTATATTTATGAGTGTTATGTGGTTCCTTCTCCACTCCCACTGCTTCACTTGACT
AGCCTTAAAAAAAAAAAGTGCCTCTGTTGTCCAAAGATGAAAACTTAGGTATCATTAGAGGTAATAACTG
CAGTGGGTCCAAACACATGAAATAGTGTTTAAGTTGTGACTTCATAATGACACCAAATAAAGTAAGGTAA
TTGGTCATCTTTGAAAGATGCTAGAGAACCAGTCCATTACTTTGAAAGCTGACAAAAGGAAAGGATCAAG
GAATTACCTTGCATTTCCTGTGCAGTTTGCATCAGGGTAACATGATAGTGGATACGACAAAGTTTCCCTT
TATAAAGATGTTTCAACAAACAAATAAATAAATAAGGAATGATAAGACTGTAACCCCTAATGAATGAAAG
AATCTAGGCATTGAACATCAATAGTCGATCACACAATAAAGGGACAAGACATGATGTGCCTTCTTACAAA
GAATACACCACCACTTTGAAGTAGTCTTGCCAAAATAATCAAACCTCAATCTGATGATGCTTCTAGATCC
AATTCCCAATTTATAGGAAATGCAAACAGCAGAATAACACGTTAAACTATACTACAGTGATATAATCAGC
AAGACCCAGACTTTAGTACAACTCTGGCATAAATGATCCAGTGTCTTCAATGAATATATATTAAGGGCAT
GGAAAAGAAGGTAGGAACCTATAAGCTAAAAGACTTGAAAAAGCAAAAGCCTAAATGTAGTCTTTAAAGT
TGCCATTATTTGATTATAGAAAAGTAAAGAAGTGATTACTATAAGTATCCAAATAGGCCAAGCACAGTGG
CTCACACCTGTAATCCCAGCACTCGGGAGGCTAAAAGATTGCTTGAGCTCAGGAGTTCAAGACCAGCCTG
TACAACATAGTGAGACCTCGTCTCTATTTAAAAAATAATAAAAAAAAATGAGCCAGGCATGGTGGCATGT
GCCTATGGTCCCAGCTACTCGGGAGGCTAAGGCAGGAGGATTACTTGAGCCCAGGAGACCAGGGCTGCAG
TGAGCTATGATCACACAACTATACTCCAGCCAGGGCAACAAAATGAGATCCTGTCTCCAAAAAAAAGCCC
AAATAGTGGCTACTATTAAGAAGGGAGGGAAGGGGGTTTATATGGCATGTTGGAGAGGTTCTGGGGTAGT
TAGCAAGATTCTGTCTTCTGACCTGGGTGGTGGACTTAAGGGTTTCACCTTATAATAATTCGTATAGCTG
TATATTTCTTTTTTTGCTGTTTTCTGTTTGAATTATATGAAATAATAAAATTCTTTTAAAACTGAAATTG
TGGAAGATAATAACATACATGATGACAGACTCCACTGGATACAGCTAAAGCTGTACTCAGCAGGAAAGTC
ATGCAAATTCTGAAATAGAATAATTGGGACTAGCCCTAACAGTTATTAAAATATATTTTGTGGCCAGGTC
TGGTGGTTCATGCCTGTAATCCTAGCACTTTGAGAGGCCGTGGCAGGAGGCCAAGTTCAACACCAGCCTG
GACAATATACTGAGACCCTGTCTTTACAAAAAATTTTAAAAATGAGCCAGGCAATGTAGCATAAATCTGT
AGTCTCAGTTATTTGGGAGGCTGAGGTGGGAGGATTGTTTGAGCCTGGGAGGTCAAGGCTGCAGTGAGTC
ACAGTGGCGCCACTGTACTCTAGCCTGGAGGACAGAGCAAGACCCTGTCTCAGAAAACAAACAAACAAAT
ATATATTTTGTGAAACTTCACTAAATAACAGTGTGGTTACGTGTAGCAGAAAGTTTAATTAATGGACAGC
AAATGTAGCAGGAGACCCAAATGTAAAAAGGAATTTACGATACAAGTGACATTTCAAATATATATAAGGA
AGACATGGCTTATTCAATAAATAGGATTCATTAAAGTTGGAGCAGTATCTCATATCTAAATAAGTACTGA
ATGGATTGGTGGGTTAAATGTAAAAAATGAAATTATAAAAGTACTAAGATAAACACAAAACATAGAAAAA
AAAAGTGCTAGGAAACATGGGAAATTTAAAAATCTGAAAAGTTGGGAACTAGAACATAAAACCCAGAAGT
CATAAAAGAAATCACGGAATTCTGTTCAATCTCAAGTCGTAGACTTCTTTTTCTGCCCCGTATTCTCTGT
GTTTAATATCCTATGATCTTGAGGCACTTAATTTCTCTGTATTTTTTCCCATGTCTATACTTAAATATTG
AGAGGATCAAATGAGATAATATGTAGAAATGCTTTGGAAAGTTGTAGTAGGTTAAAAAATAATAATAATG
TTATTTATACACTTTGCTCCTGTCATTTTTTCTGTGCCTTCTTTTGGGAGGAAAGGACAAGGTGTGGGGG
TGATAACTAATTAAGGAGGTAATTTTGGCTGTGTGAGGTGGTTCACGCCTGTAATCCCAACTTTAGGAGG
CTGAGGCAGGAGGATCACTTGAGCTCAGGAGTTCGAGACCAGGCTAGGCAACATTGTGAAACCCCATCTC
TACAAAAAATACAAAAATTAGCCAGGTGTGGTGGTGTACACCTGTAGTCTTAGGTACTGGGGAGGCTGAG
GTGGGAGGATGGCTTGAGCCTGGGAGGCGGAGGTTGCAGTGAGCTGTGATTGTGCCACTGCACTCCAGTC
TGGACAACATAGCAAGACCCTGTCTCAAAAGGATTTTTGCTGTCTGTTCTTTGCATGTCTAATTATGTTT
TTATAAAAAGTTTGTTACAGCAATACATTTTTACCCTAGAGTAAGCATTTGTAAGGGGATTAGCTTAATT
TCAGATAGAGTAAACCAACTTTTAATCAGTATTTTTTTTTATACTTACATGATCTCTAACCACACTGTTG
TGTTGGAAATTTTGTTAGTTTTGCGGGTTAATTGTATATGTGACCATTTCCACTTCTTTAAACATAAATT
TCAGTGTTCAAGAATACTTTGTTCGCAAATCCAATGCAAAGAAAGGCATGTTTGCCAGTACTCATCTTAT
ACTGAAAGAACGTGGTGGCTCTAAATATGAAGCTGCAAAGAAGTGGAATTTACCTGCCGTTACTATAGCT
TGGCTGTTGGAGACTGCTAGAACGGGAAAGAGAGCAGACGAAAGCCATTTTCTGATTGAAAATTCAACTA
AAGAAGGTTAATACTTTTATTCTGTTCTTTTATATATATTTACAACTAGATGGTTTCTGGGATATAAATG
TGCCAATGCTTGTGTTTAGGAAGGGTCATTGATTTTTGCTTTTCACATTGGGTGCAGTGAGGGTCTCAGT
GTTCGAAGTCTACAGATGTAAGCTTGGCTCCTATTTACTGTTGATTCTTTCTATGGCTCATAGTAACACA
CTATATAATTAACATGCTATCTCTCCTCTTATCTTTGGGCTTCTGCATGTTTCCTGTAGACCACCTCACA
GTTGCCTGCTCTTCCTGCTTATCTTTCAGTTCTCAGCTAAGATACCACTTTCCCTGGGAAGCCTTTCATT
TTTTCTTTTTAATTATTATTACACTTTAAGTTCTGGGGTACATGTGCACAATATGCGGGTTTGTTACATA
TGTATACATGTGTCATGTTGGTTTGCTGCACCCATTAATTCGTCATTTACATTAGGTATTTCTCCTAATA
CTATCCCTCCCCCATCCCCCCATCCTACGACAGGCCCCGGTGTGTGATGTTCCCTGCCCTGTGTCTGTTC
AATTCCCATCTATGAGTGAGAACATGCGGTGTTTGGTTTTCTGTCCTTGTGATAGTTTCCTCAGAATGAT
GGTTTCCAGCTTCATCCATGTCCCGACAAAGGACATGAATTCATCCTTTTTTCTGGCTGCATAGTATTCC
ATGGTGTATATGTGCCACATTTTCTTAATCCAGTCTATCATTGATAGACATTTGGGTTGGTTCCAAGTCT
TTGCTATTGTGAATAGTGCTGCAATAAACATATGTGTACATGTGTCTTTATAGTAGCATGATTTGTAATC
CTTTGGGTATATACCCAGTAATGGGATCGCTGGGTCAAATGGTATTTCTAGTTCTAGATCTTTGAGGAAT
CGCCACACTGTCTTGGGAAGCATTCTTGACTCACCAAGGATGGCCTTTGTCTTGGTATAATGTTTTTTGT
TTCACCATGACACTCTGTATCATCCCTTTTATCTCTTACCTCAGTGTTATAATGGTTGTTTATTTTGTGT
CTTCCTGCTAAATTTAAAGTTCCAGAAAGGCAGTGAACATGCATGTCTTGTGAATTCTGTATTTCCCATG
TCTAACACTAGGCCTGGCATGGAGTAGCTATCTAATAAGGTTTTTTGTTAAATGTTGAAAAAAGGTGGAA
GGCATAGGTGGAAGAATACAGTACCCTCTCTTGTAAGATAATAGCATCCATGGGCAAACTTCAGGGACAT
ATCAGCAGCTTGGGAATACCTCTCAGAGTCATTACTTATTTTGCCAGACAGACTGGTAATTTTATGACTG
AGGCTAAGTGATGTTCAGGTCATATTAAAATTTGATTAAGGACTTTCCAAAACTAGACTGTAGTTAACTC
TGAGTACCTGTAATTGAGATTTTTCTTTTTACTCTGTCATTGACTAGAAAGACATTCACTGATAACAATT
TTTGGCAAATAGGCTTATTGAGACATAGCAAGATGAAACAGGAAATCTATTATTATTTTTTCAGTTAATG
TGCTTGTCTAACTCAACATATATTTAATGCATAATCTGGTTTGTTTCTTGGAAAGTAGGTATATAATTTT
GTGATTTAAAGTGTACCTGAGAATTGACATGAAAACTAACAGATTAGGATGCCGAGGTAGATGGATCTCT
TGAGCCCAGGAGTTATGCAAGAAAGAAAGGAAAGAAAACAGTATAAAATATTACTTCTTGTGTACCGGGA
AAAAGTGGGCATTAGTGGCTCATATTTGTTTCTTTTCATTTTAAATCTCTAGAACGAAGTTTGGAAACAG
AAATAACAAATGGAATCAATCTAAATTCAGATACTGCAGAGCATCCTGGCACACGCCTGCAAACTCACAG
AAAAACCGTCGTTACACCTTTAGATATGAACCGCTTTCAGAGTAAAGCTTTCCGTGCTGTGGTCTCACAA
CATGCCAGACAGGTCGCAGCCTCCCCAGCAGTAGGACAACCACTTCAGAAGGAGCCCTCGTTACACCTGG
ATACACCATCAAAATTCCTGTCCAAGGACAAACTCTTCAAGCCTTCCTTTGATGTGAAGGTAAGGTTTAT
AGGCAGTGGTCCCAGAGTTAACAGTTTTACCGTTTATAAAGTTAAAGCACCAAATTTGGCATCACCTCTG
AAGTGTAGTAATAAATTTTCCAGAGACATTGGCAAGTAGATTTATGTTACTGAGTTTGAACGTTTTGAGT
CCCAGCATACATTTAGGGTTAAGGTAACAGCTATAGATAAATATGGTGTGAAATTAGGACATCTGGGTTC
AAGTACTGGATCGATCTTGAACTGTATGACTAGGCCAATCATTTAACATATCTGAGCTTCAGTTTGTTTT
TTCATACAAAGAAGATAATATTTGCTCTGAAAATTCATAGATTATAAAGATCAAATGATGAAATCACTTT
ATAAACTATAAAATGTAAACACACTGATACTTTATATATTGGTTCTCAGAGTACACCCGGGATTTGCCTT
CTGAAAATGTGTGTATATCACCTTACTTGAATTGTTTCCTGTAAAGACATGAGTTAAGTCTGTGTTTTAG
TTTGATCTCGTGAGCATTGTAGTATGGCTCCCTTTCTGAAAAATGCATTTGTAAATCAGGGGCTAATGTA
ATCTTCCAAATAATTTTCTTTGATTTTCCAAAACTGATTTTCATCATTATTTCTTTAGTAGCTTTTGACC
TCTTAAGTGGCTGTTCAAACCCACTGTCTATGATTTCTTTATCCACGAACCTTTGAAGAATTATTTTACT
CATTCACGTGTTGATATTTTTGCTTGTAGGGTATTTAGAAGCAATATTTTAATAATTTTTATTGTGTTTT
GGAGTGGACTTACTAAAAAGTTTATAGATTATTCATATTCAGAAAATAAATATTAATTGATATCCAGGAC
AGTTTTCCCCAAGCTAATTTATGCCTTTATTTTTGGTCAGAATTCCAGTGACGCACATTAAAAAAAGTGT
GTCTTTACTATTAAACTCAAAATTCTAAGTTTTCACATGAAATCTCTTCTGAGGTAACATTTTGTACTGT
GATTAATACTCAGGTCCATATCCCTTAAGACATAGAGGAAAATCAGTCTTGAGAACTGTGAGGTGTAGGA
TCAAGCCTCTTCTTTGCCTCCTACTGCCTTTTAAAAGCAAGTCAACCTCTTTCGGCTTCATTATTCTCAT
CTATAAAATCCAGATGATCTCTAAAGTGTCATAGCTCACAGACTCTCTGAAGAGCAAATGTAATAATATT
GGTGGGCAAGAAAATAGTAGAGATATTTTCAGTCATTGAGACCATTCTTATATAGATACATTGCAATAAA
AGTTTTGCAGTGGAAAGCTGATAGTTCTGAATAGCCTCAATACAAAAAACACATTAAAGATTTTATTGTT
TAGAGTCATAGTTTATTGAGCAGTGTTTTTCCTCTTTTTTCTTTCTTTTTTGATGGCACATAAATAATGC
TTCATCTTATAATCTTATAAGCATTATAATGCTTCATCTATAATCTTATAATCAGATGATAATCTTATCA
TCTTCAAACTGATATAATATGGTATGTTGAATTCTTTACTTAAAACATGAAAGGGCCCATGGAATAATCA
CAAAATTGACCATCTATTAGGCCTCACCCTTGCCCCCCTCAAAAAAACTCACCACTTATTTAAACTTAAT
TTTTAATTAATATACATCAGAGCAGTACAATAAATATACTGCTAATGAACTATCAGGGTTAATCATTGAG
AAGCCACTATACACGTTCAGATAGAGACCATGGTCAGTACTGTAAACCCTTCCTTGTGGCTCCATCGCTG
CTTCCTCCCTAAAGTATACTCCTGACTTCTAATAACTATAGTTTTGTTATGCTTGTTTTTGACTTTTAAA
TATGTGGAATCATAGTATGTATTCTTTTGAGTCTGTTTTTTTTTTATTGTTCTTTAACCCTTATTTAGAT
TTGTCTGTTTTGCTGCATGCAAATATAGTTCATTTATTTATTCCTTTGTATGGATATACAGAATTTACAT
TTCCAGTGTACTGTTGATGGGCATTTAGTTGGTTACTAGTTTTTTGCCCTTACAAAATAATTTTACTGTG
AACATTCTTGTGCGTGTCTCTTGGAGTCTATGTGTGTGTGTTACTATGTGGTATATACCTGGAAGTGAAA
TTGTTGGGTTATATTTCAGCTTCAGTAGAGAATGACAAATTTTTTGCCAAAGTGATTATACCAGTTCATG
CTTCCACCAGTAGTGTGTTGAGTGTACCCAGTGTTCCTCATCTTTGCTGATACTTGCTATTATAAGTTGT
TTTATTTCACTTTTTACTTCAAAATAATTTCTAACTTACAAAAAGTTGCAAAATAAAAGTGGTACAAGGG
GCTGGGCGTGGTGGCTCACACCTGTAATCCCAGCATTTTGGGAGGCTGAGGTGGGAGCCTCACTTGAGAC
CAGGAGTTCTGGACCAGTCTAGGCAATATAGCGAGACCGCCATCTCTAACAAATTTTTTTTTTAATTAGC
TGGGCTTGGTGGAGTGCACCTATAGTCCTAGAACTCAGGAGGGTGAGGCAGCAAGATGGCTTGAGCCCAG
GAGTTTGAGGCTGCAGTGAGCTATCATCCTACCACTGCACTCCAGCCTGAGTGACAGAGCAAGACCCTGT
CTCAAAAAGAAATAAGTACAAGGATGGCTGGACATAGTGGCTCATGCCTGTTATCCCAGCACATTGAGGC
TGAGGTGGATCACTTGAGGCAAGGAGTTGGACACCAGCCTGGGCAACATGGTGAGACCCCACATCTACAA
AAAGTTGGAAAAATTAGCTGGGCATGATGATGCATACCTCTTCTTACTTAAATAGTATTCTTTATTATTT
GTCAGTTTTAAATTATATTTTTGACTCCCACTAATTGCCTGTATAGAAGATCTTATTCTCTTTCCCCCTT
TTCTCTCTTCTATTTTTCATTGCATATTTTTTTACTTTGTCAGGAGGGAAAATATATTTTTTCAACCCAC
AGACCCCACCCTCGTTTTGGTCTTAACTCTACAATCGAATATATGAAATGTGTACTCTTCTTTTTGAAGC
TTACCTGTCACTTTTGGTAGGATGAAGTTCATTTTCTAGTAGGTTCCTCAGGAATCTGTGTGTACACACG
TGTACAGTATTCCTTGAGTTCTGCATGTTCATAACTTTTACTGTGGCCTTGCTTCTTCTACAAATTGTTT
GGATGTACTATTCTTAGCTTGCAGTTTTTTTTCTTGAGTTTCCTGAAAATGCTCTTCCACTACTGCCTTG
CTTTGTATGTGTTGTTTTTGAAAAAAAATCTGATGCCAATCTAATTAAATTACTTGTCTTTGGAAGTTAT
TTCATCTTTTTGCCTGGAGGTCATGTGGATTTTTCTTTTATCTTTTATCTTTTTTTTTTAAGACAGAGTC
TCGCTCTGTCACCCAGGACCCAGGCTAGAGTGCAGTGGCACAATCTCAGCTCACTGCAACCTCTGCCTCC
TAGGTTCAAGCGATTCTCCTGCCTCAGCCTCCCGAGTAGCTGGGATTACAGGCATGCGCCACCATGCCCG
GCTAATTTTTGTATTTTTAGTAGAGATGGGGTTTTGCCATGTTGGCCAGGCTGACCTTGAACTCCTGATA
TCAAGTGATCTGCCTGCCTTGGCCTCTGAAAGTGCTGGGATTACAGGCATGAGCCACAACACCTGGCCTT
TTCTTTTATCTTTGAACTCAAATAGTTTCACGAGGATGTCTAGATGTTAAGAATTCTCAATCATTTGAAA
TCAGTTTTCCACAATACATGGTAGGTTTTAAAATTTGATTCTTTTCAAATTTTTGGTCTGAGAGATGAGA
AGAATGGAGTTGCCAGTCCAAACTGAGTTGAACAGAACAGGTTGGGATTGGAGCCTAAAGATCTAGAGTT
CACATGTTAGACACATTAAGTTTGAGATGCCTATTAGATATCCAAGTAGTTGTTAAATAGGCAGTTGCAA
ATAACGCTGGAAGTTTGAGGAATGGTTTGGGCTGGAGATAAAAATTTGGGGTTCTGAGTTTGTAGGTAAT
GTGTAAAGCCATGTGAGAATAGATGCCAAGAGAGTAGGAGAAGGCAAAGAAAGGAAAAAGGTTTTGTTTT
GTTTTGTTGTGTTTAAGACAAGGTCTCGCTGTGTCGTCCAGGCTGGAGTGTAGTGGCATGATCACAGCCC
ATTATAGCCTTGACCTCTGGACTCAAGCAATACTTCCACCTCTGCCTTCAAAGCAACTAGGACTTCAGGT
GCATGTCACCACACCTGGCTAATTTTTAATTTTTTTGTAGATACGGGGATCTTACCACATTGCTCAGACT
GGCCTTAAATTCCTGGACTTGAGCAATCCTCCTGCCTTGGCCTCCCAAAGTGTTGGGGTTATAGGTGTGA
GCCACCACGTCCAGCCAAAGAAAAGAGCTCTAAGCAACACCTTGAGGTGCTCACCTTTAGAGGAGGAACC
AGCAAAGGAGACTGAGAAGGAGCTATAAGAGAAGAGGAAAGCCTAGAGCTTATTTCAAAGAGCCAAGTAT
TTCAAAGAGGGAGGAGGGGTTAACTATGTCCAGGACTGCTCTAAGATAAAGAAAGGTGAGAATAGAAAAT
TGGCCAGTGGATTGAGCATTATGAAGGTCAATGGCGTCATCATAACAATAAGGGTTTTGGTGGCATGATG
GAGGTGGAAGCTGGTGTGGGTTCATAAAGAAATGAGAAGAATCGGAGACATTGAGTGTAAACAACGATTT
TTGAAGGAATCTTGCCATAAAGGGGAGCCAAGAAATGGAGTTGTAGTTATGGGGCAAAGTAGGGTCAAAA
GAAGGTTGTTTTCTTTTTAAGGTGGAAGAAATAGCCAGGCTCAGTGGTGCATGCCCATAATCCCAGCACT
TTGGGAGCCCAAGACGGGAGGATCACTTGAGTCCAGGATTTCAAGACAAATCTGGGCAACATGGCAAGAC
CCTGTCTCTACAAAATACACAAAAATTAGCTGGGTGTGGTGGTGCACGTCTGTAGCCCCAGCTAGTTGGT
AGGCGGAGGCAGGAGGATTGCTTGAGTCCAGGAGATAGAGGACGCAGTGGGCAGTGATCATGCCGCTGTA
CTCCAGCCTGGGTGACAAACTGAGACCCTGTCTCAAAAAAAGAAGAAGAAATAACTATATGCCAGTGTAA
CTGCCATTAGAGAGCCAATTGATGATGCAGATGAGAGAAGGAAGACTTGCAGGAGGCTTGAGTCTGAGAG
GCATTGGGATCTACTTCTTAAGTAGAGGAAGGTACTAGTGGCACGTGTGCTGAAGGCAGATAGATGTGGT
ATGGGAGCTTCTGATTTTTATATAAGGAATCCTCTAAAATGCTTTGAAGAAATTTTAAAAAGTTTAGCAT
ATAGAAAGACTTACCATGTTCTTGCAAAATAGATTTAAACATTGTCAAGATCTATAAAGGTGTATTAATC
TGTTTGCACATTGCTATAAAGAACTACCCCAGACCGAGTAATTTATTCTTATTTTTATTTTTTTATTTTG
AGATGGAGTCTTGCTCTTGTCACCCAGGCTGAAGTGCGGTGGCATGATCTCGGCTCACTGCAACCTCCGC
CTCCTGGGTTCAAGCAATTCTGCTGCCTCAGCTTCCCAAGTAACTGGGATTACAGGCGCCTACCCCCACA
CCCGGCTAATTTTTGTATTTTCAGTAGAGACGGGGTTTCACTGTGTTGGGCAGGCTGGTCTCAAACTCCT
GACCTCAAGTGATCCACCCACCTCAGCCTCCCAAAATGCTGGGATTACAGGCGCGAGCCACCACGTCCAA
CTTTATAAAGAAAAGAGGTTTAATTGACTCAGTTCCACAGGCTGTACAGGAAGCACCACTGGGGAGGCCT
CAGGAAAATTACAATCACGGTCGAAAATGAAGGGGAAGTGAGCACGTCTTTCATGGCTGGAGAAGGAGGA
AAAGATAGCGGGGGGAGATGCCACACACTTTTAAACAACTGGATATCAGCCAGGCGCAGTGGCTCACGCC
TGTAATCCCAGCACTTTGGGAGGCCAAGGTGGGTGGATCACCTGAGGTCAGGAGTTCAAGACCAGCCTGG
CCAATATGGCGAAACCCCATCTCTACTAAAAATATAAAAATGAGCCGGGTGTGATGGCATATGCCTGTAA
TCCCAGCTACTCGAGAGACTGAGGCAAGAGAATCACTTGAACCAGGAGGCAGAGATTGCATTGCACCAAG
GTCGCGCCACAGCCCGGGCAACAGAGTGAGACTCCATCTCAAACCAGATATCATGAGAACTCACTCATTA
TCACAAGAACAGCAAGGGGGAAATCCGCCCCCATAATCCAGTCAACTGCCACCAGGCTCCTCCTCCATCA
TTGGGGATTACAGTTCAACATGAGATTTGAGTGGGGACACAAATCCAAACCATATCAAAAGGTAATGTAG
GTCCTAGAAAAAAATGCAAGCAGAAACAATATTAAAAAAAATCTGAAAAGTATAATTGCAGGTAGGCAGT
AGCCCTACCGTTTATTAAAACCTCCTATAAAGTTGCAGTAGTTAAAACAGTGTGGTGTCACGTGAATAAT
CAAGTTAGTGTATAAGATTATACTCCACAAATAGGGGAATTTATTATGTAATAAAGGTGGCATTCAAATT
GGACAATTAGGTAGCCATCTGAAAATACGTTTGTAATTAAAGCTCACACTTTACATCAGAATACTTTTTA
GATGATCAACTTTTGGGGTTTTTCTTGAAATAGGATCTCACTTTATCACTTAGGCTGGAGTTCAGTGGCA
TCATCGTGGCTCACTGCAGCTTTGACCTCCTGGGCTCAAGCAGTCCTCCCACCTCAGCCTCTGGAGTAAC
TCAGACTAGAGGTACATGACACTGTGCCCAGCTAATTGTATTTTTTGGAGAGGTAGAGTCTCACCATGTT
GCTCAGGCTGGTCTAGAACTCCTGGGCTCAGGCAGTTCTGCCTTGGCCTCCCAAAGTGTTGGAATTACAA
GCGTGAACAACCACACCCAGCCTAGATGATCAGATTTAAACATAAGAAATGAAGCCATAAAAATTCCCAA
ACATGTCATGAATTTGTTTCTAAAATAATCCTAGTGTGGGGAAGGCCTAACTATAACATACACTCCAAAA
GAAATAGAAGATTCGTATGAATTTGACAGCATTAAAAATTCTCTAACTGGGTGTGATGGCACATACCTAT
AGTCCCAGCTGACAAGGGAGGATTGCTTGAGCCCTGGAGGCAGAGGTTGCAGTGAACTAAGATTGAGCCA
CTTCAGTCCGTTCTGGGCAAGAGAGCAAGACCCCGTCTCAAAAAACAAAAAAATTACAGCGAGTCACCGT
GACTCACACCTGTAATCCCAGCACTTTGGGAGGCCAAGGCGAGAGGATTGCTTGAGCCTAGGAGTTTGAG
ACCAGCCTGGGAAAGACGGCAAGACCCTGTCTCTATGAAAAATTTTAAAATTAGCTGAGCACAGGGTTGT
GCATCTGGAGTCCCAGCTACCTGGGAGGCTGAGGTGAGAGGATCCTTGAATCCAGGAGTTAGAAGTTACA
GTGACCTATAATCATGCCACTGTACTCCAGCTTGGGTGACAAAGCAGGACTCCATCTCTGAAAAAAAAAA
AAAGCTCCATGGCAAAAATTTGACAAATGATAAATTGAAAATAAATACGTGGGTATTATATCACAGTTGA
TACATGCACATATAAATTCATACAAATGAATATAAAAGTATATTCATACAAGCGAATATATTAAAAAAAA
GACTAACAACCCAGTAGAAAAATGGGCAAAGGATAGGAGCAGATAGTTATCACAAAAAATTAGATATAAA
TATACATATAGCCAGGCGCGGTGGCTCACGCCTGTTATCCCAGCACTTTGGGAGTCAAAGGCGGTTGGAT
GATGAGGTCAGGAGTTTGAGATCAGCTTGAGCAACATGGTGAAACCCTGTCTCTACTAAAAATACGAAAA
ATTAGCCAGGCATGGTGGTGCATGCCTGTAGTCCCAGCTACTCGGGAGGCTGAGGCAGGAGAATCGCTTG
AACCCAGGAGGCAGAGGTTGCAGTGAGCCGAGATCGCGCCATTGTACTCCAGCCTGGGTGACAGAGCGAG
ACTCCGTCTCAAAAATATATATATATATACACACACACACACATATAAAGATATTTAACCTCATTCAGTA
TAAAAGAGTAGAAAATTAAAGCATACAGATAAAATTTTTTAGTCTTATCAGATTGTCAAAAAAGAAAAAT
CTGATTACTGTGATATCAAGCACGTTTGGAAAACACACACACTCATACTTTGCTCATGAAAATGTCAACA
TTGGTAGATACATGTTGGTCTTGTTTTTTTAGTTTTTTTAAAAAGAAATTGGGTCTAACTGTGTTTCCCA
GACTCGTCTCAAAACTCCTGGTCTTGAGCTGTCTTCTTGCTTCAACTTCTCTGGAGTAGCTGAGATTACT
GACAGGAGCCACTGCACTGGGCTGGTAGATACATTTTGCTTAAAAGGTTTCCTTTACTCTTGTGTGTGCA
AAATGGTTTGTGTACAAGGTTATTCATTGCATCATTATACTTGCAAAAGACTGTTAACCGAAAGCTTATC
AGTTGTACACAGGTTAAATACATTCTGATACATACATGGAATACTATACACATGCAAAAGAGAATGAGCA
AGCCATTTGGACAACAGTAGCTTCCTTTCAGGAGGGAACTGTTGGTGGCTTTAGAACAGGAATATATTTT
GAACTTTTGAAGATTTTTTTTTTTTTAATTCTCAGCTCATTTTGACTTTCTAACCTTCCTTATAATCCAT
TTCTTGATTATATCCGTCGTTGCTTATTTGTTTCCTTCTGGGTTTTTTTGGTTTGTTTTGTTTTCAAATT
CAACTCTAAGATTCAGGTAAGAGACAAGCACTGCTGAAAATGTAAGCTAGTTAGTCTCTTTGAACCAATT
TGCTGGACAGTATGGAATTTTTGTTCACAGTATATGGATTTGATTATAGTTTATTCTTTTTCTTGTTATT
TGGAGGAAAACTAGAGGAAGTTAAACTGTAATCCATTAATTTTGAGGATTCTCCCCCTTCTCTCTCTTTA
CCCCTCTCTCCTCCTCTTTCTTTTTCCTGTCCTATTAACCCATCTTGCTTCTCTCTCTCTCTCTTCTTTT
CTCCATCTCTCTCTCTCCTTCATATGTATAGGATGCACTTGCAGCCTTGGAAACTCCAGGACGTCCCAGC
CAACAGAAAAGGAAACCGAGTACGCCACTCTCAGAAGTTATTGTCAAAAACTTGCAACTTGCTTTGGCAA
ATAGCTCTCGAAATGCTGTCGCTCTTTCTGCCAGCCCTCAACTGAAAGAGGCCCAGTCAGAGAAGGTAGG
ATTAGTTCTCTTATTAAAAGGAAGTTTTAGATAACATTTCAAACAATAATGACTGATCTGTTTCTTTTAT
TTTCCATGGACAGGAAGAAGCCCCAAAGCCACTTCACAAAGTAGTGGTATGTGTTAGTAAAAAACTCAGT
AAGAAGCAGAGTGAACTAAATGGGATCGCAGCCTCTCTAGGAGCAGATTACAGGTAGTTGGCACATCTTT
CTGTTTCATGGTGATATGACTGAAATTTATAAGGAAACCTAAAGCTATTGTGAGCATCTACAAGAATACA
TTTATTTTGTTTCATGCATCTTTATCTACAAAATCTCTCCAGTTGATTTTTAATTTACCACATCTGTTGA
AAATAGCCTCTCACATATCAATTCACTCTTATATGTATATAATGTATATTTTTTAGGGTATGCACAACAA
ATATTGGAGAGCCTATTTTATGCCACTAACTATTCTAGAAACCTGGGATCCATGAAGGATCAGTATAGTC
TAAAAGAATCCCTGTGTTCAAGGAGTTTAGAGTCTATTAATAGTAATACATCTGGATCTGAGATTAAGTT
TATTTTTACTAGTGATTTTTCAGATTAGAGCCCTCTGGGAGTAGTACTAAGTACTCTGGCAGCCCTCCTT
TATTAAGATAGTCTTTTTTGAGCATGTTTTCATTATCTTTTTTTTTTTTTAAACTTTGGAGACTTAGATT
GTCAGATTTTTCTTCAGTCAACCTGACAGCCTAAGCTGAAAACAGATCTCTAGCACAAATGGCATCAGTA
TCCAGAATTGAGAATCTCTTACCTATTTCAGCCAGTCCTGAGTTGACTGTACTCCTCCACTAGCTGTAAT
GCTTGCTTTGTTGATAACTTGTGTTCAGCCATTCATAGATATCATAAGAATTTGTTTAACAAATGATGAA
CCATTGCAGGAATATGATTAGTTTCATCTTCCCCTTTGGTAACTCTTATTTCAGTTTGTTTTCTATTTGA
TAAAGAAATTAGGACAGGAAAATATAGGAGTAAAATTGATCTGTAATACATATAAGCATCTTAGTGTTTG
GTACATCCATTTGAAATAGTGAAGCTCTTCTACGGTATATTTGTATTACCTATTATAAATTGTTAGGTAA
TCTTATCTGTTATTATCTAATTACCTATTATGTTACCTATTGCCAGTTGCTGAGCATTATCTCGTTTCAT
TCTCATAGTAGCCCTACAAGGGGTGTGTGTATATTTTTTTTTCCTCAAAATTGAATGTTTCCTTGTTTAT
TCTGTTTTGATGTAGACATGATATAAGACTTTCAAGGGAACATATAGGATGTATGTTTTTATATGATTTT
AAATGCCAGTCCTCTCAGTATAAAAAATAGAATTTATTACTGAAATTCCTAAACAGAATTTAACCATTGC
ATTGCATTAAAGTTTATATTAAATCTTTTCCTGAAATGGATTGGTAATTGATAAAGGGAAGAGCCTTATA
ATATGCAGAGGTTAGATATTTATATATGCATAGAGTAAAGCAGTTCTCAAGTGAAGTTGTTCCAAATGTT
CCCTTCCATCATCTAGGACCAGACTGTCCCAAAGAGATCATCGGATACTGTCTTCTGTCTTCAGACAGTT
AAACATGTAGGCCCGATCTTTGCAACAAATCAGATGCAGCATCTTCATTTTATTACAAATATTTGTGTAA
CATTCCCTCTATTAAAATGAATATAAATATAACCTACCTACTTTTATAATTTAAAAAAGTAGCAGACAGT
ATAGCACTCTGTGATGCATAGGAGAAATAGAAGGTGCATAATATAGAAAAGATATATGTTCAGTTATGTA
TAATGCCTAAGGCTGACTACACTAGAAAACAATGGAGTATTAATACGTTTGTATACCTATGTAAAGGCAC
CACAAATCCAACAGTCCCAAGGCTGAGACCGATCTGGGGACTCAGATACCATGAGTAGCAGTGGTCTTCC
ATCAATTTATACAGTAGTCACATTTTTGTAATATTAGTGTATATTAAAACTGCAAAAAAATAACTTGTTT
ATATGTCGAACAGGTTCTAGACTCAACTAATTATAAACAACTTTTTTTTACATACGTGAATTTGTCGGGA
CATTAGAATATCATGCAAGATATGACATAATTATATGTATGACTGTTCCCCAGAATTTCCAGCATCCTGT
ACCTTACCCACTGCATGCTAGTAGCACCCTCCAACATACAAACACACATTGTAACAACCAAAATCACCCT
CACAAATGTCCAAAATAACCCCTGAAGTAACACTTTTGCTGAAACCGCTGATCTAGACCAAGGGTGTATG
GGCTGGTTCATCTGTTTTTGTAAGTAAAGTTTTATTGGAATACAGCAATGCCTTTTCACTTACAAATTGC
CTATGACTGCTTTCACATACAAGACAAAAGTTGAGCAGTTGCAAAAGAGATTGTTTGGCCTGCAAAGTCT
AAAATATTTACTATCTGGCCTTTTAAGAAAAAATTTGCCAAACCCTGATGTAAACCATCTACAACCACTG
ACAGTCTGTTCTTGAATGGCCTCTGAAGACTGAAACTCATGGTTTCCATATTGTCATAGTGGAGTTGACT
GGTAAACCAAATTGATTCAGGCAAAAATACTCCTCTGTTTTTCTGAATTATCTTGATAATATACCACTAC
GCAGAAAGGGAGACAGTTATTCTGAGAACTAACAAATTAGGCAAGGTGTATTTTTATTGTTATGTTTTAG
GATACATGGTCAGTAGGAAATAGAGATTTTTATAATATAACTTAGGAGGCAATAGTACTTCTTACTTCTT
TTTCTTAATTCTTTTTTTTTTTTGAGAGGGAGTTTTGCTCTTGTTGCCCAGGCTGGAGTGCAGTGACATA
ATCTCGACTCACCACAACCTCCACCTCCTGGGTTTAAGCGATTCTCCTGCCTCAGCCTCCTGAGTAGCTG
GTATTACAGGCATGCGCCACCACACCCGGCTAATTTTGTATTTTTAGAAGAGATGTGGTTTCTCCATGTT
GGTCGACTGGTCTGAAACTCCCGACCTCAGGTGATCCACCTGCCTTGGCCTCCCAAAGTACTGGAATTAC
AGGCATGAGCCACTGCCACTGCACCCAGCTTCTTAATTCATTTGTCTATTTAAAAAGCTCAGAGATATTC
CTTACTTAGAAAATGGAAAATATTAAGTGTCCCTAAGGATAAAATTCTGTTTATTTTGGTGATTGTATTT
AGAAATATAATTTTTACCTACCCTTTGGTTTTTACTTGATGTTTCCCAGGTCAAGAGAATATTGAATAGC
GGCCGGGCACGGTGGCTCATGCGTGTAATCCCAGCACTTTGGGAGCGCAAGGCGGGTGGATCACTTGAAG
TCAGGAGTTCGAGACCACTGCCCAATATGGTGAAAACCCATCTCTACTAGAAATACAAAAATTAGCTGGG
TGTGGTGGCGTACGCCTATAATCCCAGCTGCTCAGGAGGCTGAGATAGGAGAATTGCTTGAACCCGGGAG
GTGGAGATTGCAGTGAACCAAGATTGTGCCACTGCATTCCAGCCTGGGTGACAGAGTGAGACTCTGTCTC
AAAAAAAGAGAGAATATTGAATAGGAAGAATTGGGAGATCATAGCATAATATAATAGTTAAGAGCATGGC
TTTTGAAGTCAAAGTTATCTCTATCATTTACTCATTGAGTATCCTTGAACAGGGTACTGAACCATGTCCC
TTAGTTCTTTTATCTTTAAGATTAATGTAATAATACCAGTGTTAGGATTGTTGTGAGGAATGGATGAGAT
AATGATGTGTAAAATATATATAGTCTGTTTTGGGGCATACAATGAATAGTGACTGCCAGCAACTGGAGGG
CAAGTTGGATGCTTTGCAATTTATTAATTTGAAATACCTTTATGGTTACTGTAAAAGTGATATTCTTTAT
AAAAGAAAATTGAAACAATTCAGAAAAGCATAAAGAAGAAAGTAATATTTTAAAGGATATTCTTCTAAAC
CTCTCTCTGTATTTATATACCTACAGATATATGCATAAAATTTGTCCAAAAAGATAGAGCCTATCATCAT
TTTTTTAACTGTTTTTTTCTTGAGGCGGGGTCTTGCTGTGTTGCCTGGGCTGAAGTGCAGTGTCTGTTCA
CAGGGGTGATCATAGCACACTACAGCCTCAAACTCCTGGCCTCAAGTGTCCTCCTGCCTCAGCCTCCTGG
GTAGCTGGGACTATAGGCATGTATCACTTGGCTTGAGCCTGTCATTTTAATGGTCTTGTTTACTCTAGTC
CGAAAGTTTTGGGAAGGTCAGAATATTCTGTTCACAGTAAATAAGAATCATCTTAAACTAATGAGGATGT
TTCTCTTGGTTATTTGTTTTTTTGTATTGGTTATTTCAGGTGGAGTTTTGATGAAACAGTGACTCATTTC
ATCTATCAAGGGCGGCCAAATGACACTAATCGGGAGTATAAATCTGTAAAAGAAAGAGGAGTACACATTG
TTTCCGAGCACTGGCTTTTAGATGTAAGCAGTGCTTTTTTCCCCCCCATCTCTGTTAATTGAAAATGTAT
ACAAACATTATTTCAGAGCACTAACTCAAGAGTTTTCACTCTCAATGGAACTAGAAGCCAGTTTAATAGT
CTTGGTTAACTGTACACATTTCAGATCTTTGGCATCAAAAACTTAACATCTTTAGTCCTTAAATACTTAA
CAAATCTTTAATGGATACTCTTTCTAATTCTCCATTTCCTTGTACTAATTAAAAATGTGATATGAATGGA
AAGTTTATAATTATTTGGGGAAAATTTAACATATCAATAGAGTTTAGATCTGTGTTTCTTTTTTTTCCTG
GGTATTCTCTGGAGGGTAATTGATACCTTTGCAAAAGCACAGGAATGGTTTCAGATTTTATACTTCAGTG
GATATGACATTTAAAACATGAGGACAATCTGTCATTTGTATGATGCAAATTTTTCAGTGTAAACTACTTT
TCAAACCAGTTTTGTGAACTTTTTGCCATATTACAGTATAATGTATTGCTTGTCCTTATCCTACTTAACA
TCATCTATTCATTAAGATTTTGTCAGTCCTGTCTTAAAGCACACCATTGTATTGCAGTGTGCCCAAGAGT
GTAAACATCTTCCTGAATCTCTTTATCCACATACTTATAATCCCAAAATGAGCTTGGATATCAGCGCAGT
GCAAGATGGCCGGCTCTGTAATAGTCGACTACTCTCAGCTGTGTCTTCAACAAAGGATGATGAGGTAGGA
GATATGGATGATACATTTACCCCCTCTTAAGCTAAAGGTATAATCATATATTGCTATGAAACCTTATACA
GTGGCTTGGTTTGAGAATTTATAATTTTTCTTTTTCAATAGCCAGATCCTTTGATTTTAGAAGAAAATGA
TGTAGACAATATGGCCACCAATAATAAAGAGTCAGCACCATCAAATGGAAGTGGAAAGAATGACTCTAAA
GGAGGTAAAATATTTTATTTTTTAATTAAAAAAATTTTTTTTGAGACAAGGTCTCAATTTTTTGTGGAGA
TGGGGTCTTGTCATCTTGCCCACACTGGTCTGAAACTCCTGGGCTGAAAGGATCCTCCTGCCTTGTGAGC
CACTGCACCCAGCTGTAAAATATGTTTAAATTACTAAATACAAGCAAAGCATATATTAGAGGATTGATTT
GTGGTCCCATGCTGATCATTAAGTGATAATTTGCAAAGTTTTATGTAGTTTAAGTGTAAAGCTTATGAAA
TTTATGAAACTAGGTTTTTATGGAGTTAGTGTGATACAGCCCTATTCCATTAAAGGTAGGTGAAGTTGGT
GATTTTTTAAAAATCTAGTCTTTGGTCCAGTTCTTTTGAGTGATTTTCAAAAAGTTTTCTTATATGTACA
ATATATTTTAACATATCCACATGGTTTTAAAGATACAAAAAGACATATGAAGAGTTTCTTCCACCCTTAT
TCTCCAGCCCCCCAGTGTTATTAGTTCCTTATGTATCCTTCTAGAGCTGCTTTATGCATATATAGCCGTA
AATATGTTTTTCCCCCATTAAAAAAAAACAAACAAACACATAGGCTGGGCATGGTGGCTCGTGTCTGTAA
TCCCAGCACTTTGGGAGGCTGAGGCAGGCAGATCACCCAAGGTCAGGAGTTTGAGATCAGCCTGGCCAAC
ATAGAGAAACTCCGTCTCTACTAAAAATACAAAAAATTAGCCGGGCATGGTGGTTGCATGCCTGTAATTC
CAGCTACTCGGGAGGCTGAGGCAGGAGAATCGCTTGAACTCGGGAGGCGGAGGTTGCGGTGAGCCGAGAT
CATGCCATTGCACTCCAGCCTGGGCAAGAAGAGCAAAACTCTTGTCTCAAAAAAAAAAAAAAAAAAAAAC
ATAAATGGTGGACTACTATACATGCTATTTCATACGCTGATCTTTTTGTTACCTTGGGTAGTGTTTTATA
TCAGTAGATAACTAGGAGCTTCTTTAGTCTTTTTCTTTCTTGCTTTTCTTCCTTCCTTCCCTCCCTTTCC
TTCCTGTCCCCCTTCCTCTCCTTTCCCTCTTCCCTTCCTTTCCCTTTCCTTCTTTCTTCCCTTTTCTTTC
TTTTTGGGCAACTGTATTGTATTCATTATGAATGGTCCCACTTTGTTTAGGCATCCATTCTTAATAGGCA
TTTAGGTTATTTCTACTTTCTTGCTTTTATAAATAGTACTGCAGTGAATAACCTTATACATGGATGTCTT
TTAATAGATTTAATATGTTCTGTTTTCCTTACTCATTCTAACCTACCAAAGCTTTCCCTATGACTTACCT
ATTTGGTTAGTCTCACTCTGTTCATTCCAAATGCATTTATTTAATACTTACTCCAGAGTTTAGGCTTTAA
AGAAAACCAAAGGTAAATACAGTTGTCTTGTCCCTTGGTATCCATGTGGCAGTGGTTCCGAGGACTTACC
TCAGGTACCAAAATCCACAGATGCTTAAGTTCCTTATGTATAAAATGACATAGGATTTGAATTTAACCAA
TGCACATCCTCCTGTATACTTTAAATCATCTCTGGATTACTTATAATACCTAATGCACTGTAAATGTTTT
GTAAATAGTTATATTTTATTTTTTTTACTGGTACTTTTAATTGTGATATTTTTCATTTTTATGTCTTTAA
AATATTTCTGATCCATACTTGGTTGAATCCATGCATGCAGAACCGGTGGATGTGGAGGGCTGGATGTATG
TTGTGGTCTGTGTTTTCAAGAGCCTTTGGGGAGTCTGGGCACGGTGGCTCACACCTGTAATCTCAGAGCT
TTGGGAGGCAAAGGCAAGAGGATTGCTTGATGCTAGGGGTTCCAGATCAGCCTGGGCAACATAGTGAGAC
CTTGTCTCTACAAAAAATTTAAAAAATTAGCTGGGCGTGGTAATGTACACCTGTAGTGCCAGCTACTCAG
GTTGCTGAGGCTGGAGGATCACTTGAGCCCAGGAGTTAGAGGTTATAATGAGCTATGACCATACCACTGC
AGTCCAGCCTGGGCCACAGAGCAAGACTCTGTCTCTAAAAAATTAAAAGAATCTTTGGGAAAAATCAGAC
ACATACACAAATGACTATAAGACATCTCAGAGTGTAGTACATACCATGAGAGTCAGAAGCAGGCAGGGAG
CTTTACAGAGGGATAATCACTACAGGTCTTGATGAAATGTCTTTGAAGGGGGCCAGGCACGGTGGCTCAT
GCCTGTAATCCTAGCACTTTGGGAGGCTGAGGTGGGCAGATCACCTGAGGTCAGGAGTTCAAGACCAGCC
TAGCCAACGTGGCGAAACCGCGTCTCTACTAAAAAATACAAAAATTAGCCCGGCATGGTGATGGGCACCT
GTAATCCCAGCTACTTGAGAGGCTGAGGCAGGGAGAATTGCTTGAACCTGGGAGGTGGAGGTTGCAGTGA
GCCGAGGTCGCACCACTGTCCAGCCTGGGCGACAGTGCTAGACTATCACCAAAAAAAAAAAAAAAAAAGT
ATTTGAAGGGCTTCCTAGTTATTTCATATACAGTGCTTGTGGTAAGGGCTGCTTTTTATGTTCTGCAGTA
TTCATAGTTTTAGTACATTACTATAGCCTGTAGTTAATATTCAGTAAATGTCAAGTCAATAAGTTTGTGT
TCAACCAGGTCTAAACAAAATAGTTAATGTAAGATATGGTAAAAAAGAGGAACTTACCTTGAATGGTGTA
TTTGTGTTTGTTTCAAGTTCTGACACAGACCTTAGAGATGAGAGAGAACTTTCAGAAGCAGTTACAGGAG
ATAATGTCTGCAACATCAATAGTGAAACCCCAAGGGCAGAGGACTTCCCTTTCAAGAAGTGGTTGTAACA
GCGCATCTTCAACCCCTGACAGCACTCGCTCTGCTCGCAGTGGACGAAGTAGAGTCCTAGAGGCACTGAG
GTGTGTACCTGTCACTGATGGCAGAAAGACTAGATGACTTGAGAGAAAGGATTTACGTACCTGCTGCTGA
CTCAATTCCATGAGCTTGGCCTAATTTAGCTTTTTTTTCTTTTTTTCCCCAATATGCAGTAAAAGCTGAA
ATTTAAATATCAAGACTAGTGAAGGGGTTATATGCCTAGGCCTTATTTAGCCCAAACAGTAAATATTATA
TTCCAGTATTATCTATTGCTTTTGGCTCTCTGCTGACTAGATTTTTCGTTCTAGCTTGTCCTTCCATTTC
TTGCTCCCAGCAGTTCTAGATTTCTTACTCGGAGGTGGAGCAGACATGGAGATTTGTCTTACAGCAGCAC
CTGCTTCACAGTGAGGCATGATTGATACTTTTTATTACCTCCGTTGAGAAAGAAATGATTTTAAGATCTA
ATTTGCCTCATTTGAAATGGATTCAGTAGGCAGTTCTCTCTGCTCTTTAGTATTAGTATTCAATAGTATT
TGTATTAAACAATAAGTACATATATATTTTTTCAGAGTCTTTTTCTAATGCAATATGTAAAATCTTAAAG
TTATTTTTGTTGGGATTTTGCATGCATTTTGTATTCTCCTTGTAAAATTTATCTTCTAGAAAAGGTGATT
ACAGTTTCTAGTGTGATGTGACTTAAGCTCAGTTGCATAACTTCATCTTTTTCGTGATAGAAGAAAAAAA
TCTATAATTGTGTGATAAGAATTATGGGACTTGTATTTTCAGTTGACTAATTGAATTAAGTATAAGATAA
ACTAGCATTTATCTTACGCTAACTGTATTACATGAGGCAGTTCATTATCTCAGTACTGAGTAAAGTAGCT
TTTTAATATGCTTCAGATCATGTTTTATCATTATAGAGAACCTAAATCCTGTACCAAATCAGAGTAAATA
AATATAGTGTGAGAATAAAAGGCTATATTCTTTATTTTTGAAGCAAAGGAACTATTTGGGGGTTCCTTTG
GTAAATTGTTTTATGGGTTTTGGTGTTTTGTTTTGTTTTTTGTTTTTTGTTTTTTTTTTTTTTGCTTCTC
CACAGTGTAAGTTTTATGGTAAATTATTTTTACTAAGAATGAATGAATGAGTGAATATAAAGAAAATTAT
AGGACTTCTATTTACTGTCACGTGGAATCATTACAATCGCACCATAAAATGCACTTCCCTGTAATTCAGC
ATATTGCTGTTCCACCGTAGACTCTGGAAAGTGGTTACCAATAGCCAAAAGGCCCTTCTGATAAGGCAGC
CTTTACAGCAAGCCTCCCATGTAATCATAGTGCTTATAATTTCAAGTAATTAAAAAATATAACTACTGTT
AAGTAAAGTAACCATAGTGTTTGAAACATTACCCTGTTTCCCTAACCTGTATAATTAAGGAAAATCACAT
TTTTAAATGTACCAATTACTTTAGTATTTTTAAAAAATTTCTTGACACTGTTGAATTTCTCCTTTGAAGT
TTTAGTTCTGTAGACATGTAGTCAAAATATGTGTTCCTAGCTAAAACAGATAACTTTTCTGGAAGAAACC
TTAAACCTTTTTAAAAAAAAAAAATGTCCCTAGGTTCATTTATTTAATATTTTTAATGCATACTGTGTGC
TAGATGGTGGGCATATAAAGGTGAGCCACAACTTTTATGGTCTAACTTATGGATTTAATGAGTTTGGATT
TGTTATTGCTGTTTATTTAAACTGTCACTTCTATTCCTTAGGCAGTCTCGTCAGACAGTACCTGATGTCA
ACACAGAGCCTTCCCAAAATGAACAGATCATTTGGGATGACCCTACAGCAAGGGAGGAGAGAGCAAGGCT
TGCCAGCAATTTGCAGTGGCCTAGTTGTCCCACACAATACTCTGAGCTTCAGGTTGACATTCAAAACTTG
GAGGATTCTCCTTTTCAAAAGCCTTTACATGATTCAGAAATTGCTAAACAGGGTAACAAGAAAGAAATCT
TTGCATTTGTTTGTATTAAGTACATGTTGACCTCTTAAAAATAAACAGATGAGCATGTTGTAGTTCTCTG
ATCTGAATGTGCAAACAAAAAAAACTTCGTAGATATTTCTGGTTTTGGTTTAAACTTGAGCCAGAAAAAT
AATTTGTAAATTGAGGTACTTCATCAGTAAATTGAAAAACATTTTCATAAATTTGGAAATTTAGAATTGG
GCGTTTTTTTCTAGACATGGTGTCTACCATTGTTCTTTTATTCTAGCTAACCCTTTTTGTGTTTGTGGAG
TTAATGCTTGGTTTTTCTTCCTATCATCTTATTTTCATTTAAGTCTTTTGAATTCTGCCTAGCACATACC
TACATTTGTTCTTTGGATTGTATGAGTTTCTTTCTGAATTCTCCTATTTTTTTTCTTCCTTGAGCTTTAC
CTTAAAAACAAAATTTTCCTATCTCTCATCTTTTTTCTTCCTTTTCTTCTTTTTCACATTGCATGATGAC
CTCCCATTAGATATATAGATTTATCTCTGCTTCTGCTTTTCTCCAAGTGTAGAATTGCTCTCATGTTACA
AATTTGGGTTACACTTAACAATGAAAACTTGGTATTTAAGGAAAATGTTGGGAGAAGCCCACAGATAACA
CACTTGCTCTGTTTAATGCTAATAAATTGCATTTTTTCTTTATTGTTATTATTGTCACATGCAGATGGGA
TGCATTTATTTATAAGTTCTGGGGATAGGATACTTTTTTGCCTGTACTTTTTACATTCCAGAGTTTGTGC
TTGACTTCATCTTGACTAAGACAGATGAGAATATTTCCTAGAATTTGTTTTTAGTCTTATTTGGATCCTG
TCACACTGCAGCATTTTATTGTCCTCAGCTGTCTGTGATCCTGGAAACATACGTGTGACTGAAGCTCCCA
AACACCCAATCTCTGAAGAACTGGAAACTCCCATAAAAGACAGCCACCTGATCCCTACGCCTCAAGCCCC
CAGTATTGCCTTTCCACTCGCCAACCCCCCTGTGGCTCCGCACCCTAGAGAAAAGGTTTGTTGATCCTTA
ATATGGTGATACAGCTTTTGCATACTGCTGTTAGGCTGTATTAGAAATTTGTTGATGTCAATATCAAGGC
TTCATCAAATGTTGAAAACTATTTTTTGCATTGTATCATTATTTGTATATTTCATAACTTAGAAGACTGA
GTAAAATACCACTATAAAAGAACTGTGTATCTGATCATTTTTAATGTTAATTTTTTCTAAAGTAAAACTT
TCATTCCAGATTATAACGATAGAGGAGACTCATGAAGAATTAAAAAAACAGTACATATTTCAGTTATCAT
CTCTGAATCCTCAAGAACGTATTGACTATTGTCATCTGATTGAGAAACTAGGTACCAGTATTTTACTTAA
ATCCAAAATGTCCCATATAATAACCATATTTGGAAGTCAAATGTAGTCAATATTAATATCATTTGTAAAG
TCTAGAAGTAGAGGAAAAGAACATGGGTAACTTGTCTAAGGCTATACATTGAGTCAGCATTGGAACTTTG
ATTTTAAAAAGCTCTAAAGTCAGTAGATCATGGGATTTAAAATTCAAATGGAATCTAGCAGTTGAGGTTT
AAGAATCTTCTGTGCTGTTTGACTATTCATTCACTAATGAAGCTTTAAAGACTTACTATATAAATAAAAC
CACATTTTAATGAACTTGAAAGGTTAATAATATCTAGAGAGGAGCTCCCTCTTGTTTTTTTGTTTGTTTG
TTTCCAGTTAGAAACAGTGTGGAGGCCAGGCACGGTGGCTCGTGCCTGTAATCTCAGCACTTTGGGAGGC
TGAGGTGGGCGGATCACCTGGGGTCAGGAGTTTAAGACTAGGCTGGCCAACATGGTGAAACCCTGTCTCT
ACTAAAAATACAAAAAAATTTGCGAGACGTAGAGGCACATGCCTGTGATCCTAGTTACTCAGGAGGCTGA
GGCAAGAGAATCGCTTGAACCTGGGAGGCGGAGGTTGCAATGAGCCAAGATAGCGCCACTGTACTCCAGG
CTGGGTGACAAAGCAAGACTCTGTCTCAAAAAAAAAAAAAAAGGGAAAAGAAAGAATTCAGTGTGGAAGA
GAGCTCCTTCTTATGTAACATTATTCTTATTCCACTGGTTGTCAGGCTCTCCACCTGACATCATTCTAGA
AGCAGCATAGTAAAAGAGCAATCTGGTATACTAGAGCAGCACTGTCCAATAGAAACAGAATGTGAACCAC
ATAGGTCATTTTAAATTCTCTAGTAGCTGCATTTTAAAAACTAAAAAGAGGCTGGGTGCATTGGCTCACA
CCTGTAATCCCAGACTTTGGGGGGCTGAGGTAGGAGGATTGCTTGAGCCTAGGAATTCAAGACCAGCCTG
GCAACATAGTGAGACCCCATCTCAAAAATAAAAAGTAAAATGAAACAAGTGAAATGAACTTTAAACATAT
ATTTATCATTCAATATGTGATCAGATAAAATATCAGTGAGTTTTTTTTTATATAAAGTAAAAATCTAGTG
TTTATTTAACTTACAGCACATCTCAATTTGGACTAACTGTGCTTCAGGTGTTTGATAGCCACAGATGTCT
AGTGGCTACTCTGTTGAACAAAGCTCTATAGAGTTAGGAAAACTATAGTAAGAGAAGTTAATCTGGCAGC
TCAGCATGGAAAGAGAAAACATTAAGAAATATTGGTGGGGTTTTTTTGTTTTTGTTTTGAGACAGAGTCT
TGCTCTGTCTCCCAGGCTTGAGTGCCATGGCACAATCTCAGCTCACTGCAACCTCTGCCTCCTGGGCTCA
AGCAATTCTTCTGCCTCAGCTTCCCAAGTAACTGGGATTACAGGTGTGCGCCACCACGCCTGGCTAATTT
TGTATTTTTGTATTTTTAGTAAAGACAGGGTTTCACCATGTTGGCCAGGCTGGTCTCGAACTGCTGACCT
CAGATGATCCACCTACCTTGGCCTCCCAAAGTGCTGGGATTACAGGCATGAGCCACCGCACCTGGCCAAA
ATAATGGTTCTTAATAGTGGCCCTGAAGTATCAGGAAAAGATTAAATGTTATTTAATGCTGTCTTTTTAA
GAATGTGGAAAACTATGATATGCTTTAAAAAAAAAAAAAAGATTTATGTCAAATACAGTTGTATACTTGG
AAAAACTTTCTCTTTTTTTTGCGGGTGAAGCTCACAGCTTTATTTTTTTTTATTTTTTTATTATTATTAT
TATACTCTAAGTTCTAAGGTACATGTGCACAACCTACAGGTTTGTTACATTTGTATCCATGTGCCATGTT
GGTGTGCTGCTCCCATTAACTTGTCATTTTCATTAGGTATATCTCCTAATGCTATCCCTCCCCCCTCCCC
CCACCCCAGGACAGGCCCCGGTGTGGAAAAACTGTTTCTTCTTAGAAACTTTACTCATACAAAACACTTT
GTTTCCCAGTTCCCTGATAACTTAGGTGAGGTTATAAGTCAGAATTGCTAAGCTTTACCCAGAACTCATG
AAAATGAAAGGCTTCAAGTGTCAGTCTCCTATAGATTTTTCCATCTCTGCAAACTTTGGGGAAGAGTGAC
AGCAATTAGCAGAGAATTGATCTAGAAGAGGGCAGGAAGCAGAGGCAGATGAGTACAGATTATGTTGGAA
TTCAGAGTCTTGTAACTCTAGAGGTTTTCCAATATTAGAATGCAAAGAGACATTATAATAGATATTATGC
CCAATCCTTCATTTTATAAATGAAGCAACTGTGGCCAACTGATATATATGTATATAAATCAGTCTATTAT
GTTGTCTGGGTCGCAGAATAAGTTTGTTATGTAGAATTGGAAATTGATGTTTATATATTAATATGGAATT
TTACCTTCTTGTCTTTTTGTCACTCTGTGATCCTGAGTAAGGTGCTCTACCAGTTTTCATGTGCATAAAT
GCAGATAATAATAGTAACCTTCCTTCATATGGTTGTGAGAATTAAATGAGTTAGTAATCTGGAGCACTTT
AAGTAGTTCCTGGCATAAAATAAATGCTCAGAAGGTGTTGCTGCTATTATTATTAGAGTCATCTTAAATC
ATGAGTTTGAATGTTTTGTTAAAATCTTAGAAAGTCCTTGATATGACCGGGTGTGGTGGCTCACACCTGT
AATCCCAGAACTTTGAGAGGCTGAGGTGGGCAGATCATGAGGTCAGGAGTTCAAGACCAGCCTGGCAAAC
ATGGTGAAACCCCGTCTCTACTAAAAATACAAAAATTAGCCAGGTGTGGTGGCGGGCACCTGTAATCCCA
GCTACTCAGAAGGCTGAGGCAGAGAATTGCTTGAACCCGGGAAGCAGAGGTTGCAGTGAGTCGAGATCGC
GCCACTGCACTCCTGCACTCCAGCCTGGGTGACAGAGTGAGACTCCGTCTCAAAAAAAAAAAAAAAAAAA
GTCCTTGATATAACTAACTCTGCCCAACCAGTGGTCAAGTTAACCTCTAAGCTATAAATCAACAGCCATG
CTTTACATAAGCTTATGTTTACTTATGCCTTCTTCTTTCCTCAAAGTCAGGCTTCATGCATTTATATCTG
AGAAGAGGTTCAATTATTGCTTTTTCCCACCTGCTGCTTCATCTCTTATTTATCTGTGTGTGTTTTCTTC
CCAAGCTTCTCTGTTGGTCTGCCGACATTATTTCCACTCATAAAAAATTTCCTCCTGTTTTAAAGTCTGC
AGATGCCCCTATCTTACTTCCATCTCCCTGGTTACCCATATTCGGTGCTTGAAAACTGCTTTTGTAATTG
AGACTTAAGAGACATAACCAACTACAGCGTGTGGCTCATGTTTATGTTCTATTTCTTTTTGTATCCTAAT
TATAACAATCCAAGTGAAGACAAACAGGAAAAATTGAACAAGGATTTAGTACTGAGACAGCAAAGAATCT
TTGTTAATCCTATTAGATCTGATAATGGCATTATGGCTGTTTAAGAAAATTTCCATATATTCTAGAGATG
CATACTGAAATACTTAGGGGTCAGTAACATGGTATCTAGAATTTGCTTTAAAATAGTTAAGCAGGACCAG
GCATGGTGGCTCACGCCTGTAATCCCAGCACTTCGGGAGGCTGAGGCAGGTGGATCATTTGAGGTCAGGA
GTTCAAGACCAGCCTGGCCAACATGGTGAAACCCTGTGTTTACTAAAAATACAAAAATTAGCCAGGCCAT
AGTGGTGTGTGCCTGTAATCCCAGCTACTTGCGAGGCTGAGGGAGGAGAATCGCTTGAGCTGGAGGTTGT
GGTGAGCCAAGATCATGCCACTGCACTCCAGTCTGGGTGACAGAGTGAGAACCTGTCTCAAAAAAATTAA
AATTAAAACAGAATAAAATTAAGTAGTTAAGCAAAAAAAGGTGGCAGAATTTGGATAATTTTAGAATATA
GGTAGCTAGTATATGTGTGGGGGTTCATTTTACTATTTTATTTACTTTTGTATTTAAAATGTTCATAATA
AACATTTTAATACATATATATATGTATATAAAGTAAAATAATTCTAAGTAAGTCAAGTTTTTTTGGTTTA
AATGGTTTGGGGCATTTTATTTATAAATAATTTCTATGCGTAGTTGAGATTACATTTTTCCTAGAAAGAA
GTTGGGAATCAATAAAGTACAATCCATTTTTCTCTGCTTAAGGTGGATTGGTGATAGAAAAGCAGTGCTT
TGATCCCACCTGTACACACATTGTTGTGGGACATCCACTTCGAAACGAGAAGTATTTAGCCTCAGTGGCA
GCTGGGAAGTGGGTGCTTCATCGCTCCTACCTTGAAGCCTGCAGGACTGCTGGACACTTCGTGCAGGTGT
GTATTCAGATTTGTGGAAGTTTATTTTTCTTTAAAAGCAAGTGTCCACCGGGTGCAGTGGCTCACACCTG
TTATCCTAGCACTTTGGGAGGCCGAGGTGGGTGGATCACCTGAGATCAGGAGTTCGAGACATGCCTGGCC
AACATGGTGAAACCCTCTCTACTAAAACTACAAAGATTAGCCAGGCATGGTGGTGAGTGCCTGTAATCCC
AGTTACTCAGGAGGCTGAGGCAGGAGAATCACTTGAACCCAGAAGGCAGAGGTTGCAGTGAGCCGAGATC
ATGCCATTGTACTCCAGCCTGGGCCACAAGAGTGAGACCCTATCTAAAAAAAAAAAAAAAGTAAGTGTTG
GCTGGCAGTGGCTGGCTCATGCCACTTTCAGCACTTTGGGAGGCCAAGGCAGGAGGATGGCTTGAACCTA
GGAGTTCAAGACCAGCCTGGGAAACATAGCAAGACTCCATCTCTAGAAAAAATAAAAACAGTTAGCTGGG
CATGGTGGTTCCCATCTGTAGTCCCAGCTACCTAGGAGGCTGAAGTGGGAGGATTGCTTGAGCCCAGGAG
GTCAAATCAAGACCCTGTCTCTAAAGGAAGAAACAGACAAATTTATTTATTTACTTAAAAAAGTAAATCT
CAGATTTCATCACATTTGCTGACTGCTCATAAAATTGAAATAACCAAATATGGCCAGTTAGAGTATACCA
GCTTTTTCAATAACAAAGCCAAACAACGAAACTTCGCAGAGCCAGGTTCACCTTTAATCACTTTTTAAAT
GATTTAATTTGAATGTTAGTGTTGTTTTTAATTGAAATAACATTTTCTTAGAAAGTTTCAAGATTATCTC
ATTGTAAGCTTTCATCTTTACTACATAGACAGATAATCAAGTTATCAGTACATACCTCCTCTGAGACTGT
GTCCTTTCATGAAGTTAGTCTTACAGCAGGTGAAATTTGTCCATCTGAATATTACCAGTTGCCAAGATAT
AGCTATCATTTTGTTTCTTAAAAATGTATGTGGTATTTTTCTGATTTCTAATTAAAAGCCAAATATACTT
GACTCTTATATGAAACTAATCTACATTTCCTCCATGCTCATAAAACAATATTTACTTAGTACTGTCTGTA
TTTGTAACAATTAAATGTATAGCTTTAACAGTTATTAGACTTTGGGCTCCTTGAGGAGCCCTGTATGTTG
CACAGTGGCTCCCCAGACTGTAACAACTCAGGTTTGTTGGACAAATTCTCTAGGAAGAAGACTATGAATG
GGGAAGTAGTTCCATACTTGATGTTCTGACTGGAATCAATGTACAGCAACGAAGACTAGCACTTGCAGCA
ATGAGATGGAGAAAAAAAATCCAGCAAAGACAAGAATCTGGCATTGTTGAGGTATTAAAACACAATTATA
TAGGTAATACAAATAGCATTCAATATTGTGACTCTTTCACATTTTTTTTAAAAGAGAATGTGCTTCAGGA
AATAATGATTCTACTTGTCTTTTTTCCCCAAATTTTTGATGGCTCTTTTAAAAATAATTTATATATGTAA
AATGTGGCCCATATTGCATTTCTTCCAAAGAATCTTTTCAGCATCTAATGTCAGGGGTTCTTGACCTGAG
TCCACAGACCTTCTCAAAGGAATTTGTAGATCAAATCTGGGAAGGCCATGTACTTTGCATGGGGAGAAAA
ATACATCCTTATTTTTACTAACCTCTAATTGAAAATTAGCACTTCCGGTCTTTTTTTTTTTTTTTTTTAA
ATTTTAATCAGCCTACATCATGCTTTGAGAAAACTAGCACTTCCTTTAATGAAAGGTCTAGGCAACAAAT
CACAACACTATCAACTGTACCTGTAATTTTGCACTAACAGAAATCACATGTTTCTGTTTTGCATTGTAAT
TGCAAGCATCTTGAAATACTGTTTGTGTTCATCAGTACTTTAGTTATTTATATTATTTGTTAACTGTTGA
TTTATCAAGAGAAACTATTAGGCCACATGTTCATGTTTCATAGTTCAGGAACACAAGTCAAAGACAAACT
TCTGTGTATTAATGATTCAGTCCAAAGAAATTCTTTATATTCCAAAATCACTTTGCACTCTGAAACATAA
TAGCACTCTTCATCTCAGATTCTTTCATGGAATCATAACTTCTGTAGAAATCAGCATGTCCTTTCTGTTT
TGGTTCAGCCACAGCAAACAGAGAGCTGCAACCCCAGGGATATAAGGAATGCTCCCACAATATGAAATCA
CAGACACTTGGCCAGAAGACCACATATCTGAGGTTTTGTCAAAGCACTGAAAGCCATATTAGTTATTGTC
TGCTACTTTAATATAAAGCACATCTATAATTACACAACAAATTTGTTTTTAATATTTTGATAACTTTCAA
TAAAATGGCTTCCTTTTGTAATCCTTTATATTTTAATTTTAACATTTTCTAAAAATCACCCATAAGGTTT
GGGAATTATATCTCAATACAGCTATTATTTAGAGTAGTCTAATGACCAGACTGCCAAAGGGATCCATGGC
TTACAAAAAGGTTAATTGTATTAGTTCGTTCTCACGCTGCTATGAAGCAACACCTGAGACTGGGTCATTT
ATAAAGGAAAGAGGTTTAATTGACTCACAGTTCCACATGGCTACAGAGGCCTCAGGAAGCTTACAGTCAT
GTAAGAAGCAAACACGTCCTTCTTCACATGGTGGCAGGAGAGAGAAGTGCAGAGCAAAGTGGGGAAAAAC
CCCTTATAAAACCATCAGATCTCCTGAGAACTCACTATCAGGAGAACAGCATGAGGGAACTGCCCCCACC
ATCTGATCACCTCCCACGAGGTCCCTCCCCCAACACTTGGAGATTATCATTCGGATTACAATTCAAGATG
AGATTTTGGGTGGGGACACAGCCAAACCATTATCAGAACCCCTGAGTTAAATAGTCTTTCATGTTGTATT
CTAAGTTCTCTGATATCTCCAAATCCTTATTAGCCATGATACCCTCTCTTTTAGAGTAATTCCACTGGCT
TTTGTTAGTTGTTATTCCCTTCCTAAATATTTTGTTTCCAGTTTGTAAACATTTTTAAGAGAAGTGAAAC
GCAGGCTTCATCTAACCTAAGGAGCTCTGCTTGAAGCTCTTCCCTTGATTAATCAAAAGAAAAAAATACT
AGAAAAGACTAAACATCAATCACACTGCTTTGTTTTAATGTTACCTAATCATTAATGGTTTTTCATTCCT
CACGTATTTCTTGATGGCCTACTATGTGCCAGATACCATGGATTCACGTTTCAGTGTTCTGGCCAGAGAT
AATTATACTGTGGCAAAAGTAAAGTTTATAAGATTTATTGACAAAACTAAGTCTACAGATAAAATCATGC
ATTATCTGTATTATTTTGTTATTTTCCAAATGATTTCACCACTGATTGTTTTTTATTTGTAGGGAGCATT
TAGTGGGTGGAAGGTTATTTTACATGTGGATCAGTCTCGAGAAGCAGGCTTCAAACGCCTTCTTCAGTCA
GGAGGAGCAAAGGTTTGTATCAAATTAATTTACTGACCTTTTTACATGAATAATCATCTAACCAAGATTG
CTATGATGAGTTCTTCATTTTCTCTACTTTTTGAAATGGTAATCCTTAAGTACACATTATACAGCTGAAC
TACTTTGGAATACTGGTAGATTGTGATACTACCTTGTTTTTTCATTAGGTGCTACCTGGTCATTCTGTAC
CTTTATTTAAAGAGGCCACACATCTTTTTTCTGACTTGAATAAACTGAAACCAGATGACTCAGGAGTTAA
TATAGCAGAAGCTGCTGCCCAGAACGTGTACTGCTTGAGAACAGAATACATTGCTGATTATCTCATGCAG
GTTTGTGAGATTCTATCCTTGACCTCCTCATTACCAGAGATACGTTTATGTGCATAGATAAGTAACTGCA
CAACAAAACCTTCAGCTCATGATTAGTAGAAGATTAGTCTCCTGTTATTCTCTTCTATTCCAGCCCTTTC
AGTTTTATTCTATTTGTGTTTTTTATTATAATGAACATATTATGTAAACATTTAGCTTGGAAGTTAAATA
TGAGAAGAGTATAAAAATCTAGGTACCTCTAAAGGCTTTAAAAATACCCTTTAAAATGTTGGAATATAAA
GCAGAAATCGATTCCAGGAGTCCAAGGAGAAACATTTATCCAAAGGTCCTTTAGTTTTCTTGACTGTTTG
CTTTCATGTACATTAAATACCTATGTTAGACACTGTGGTAAATTTAAGCATTTGCTATATGGTGGGCTTA
CTACTCTCATATTCAGAATTGGCCTGTGTTGTGTAATTTGAATGGGGTAAGAAAAATCTTCTGACACCAG
AGTGCCCAAAAGGCCATCAGGAGTCAGCAGGCATGACACATAATCATAAGGATTAGATTTCATAATCATA
ATCTATGAAGGATTTCAACTGGGATGATACAGAAATAAGGGGAGGCCAACACTCAAAGTCAGCTTTGTGA
TTTTGCTGCTGTGCTTGGAGATTGCTCAGGATTTAATAGAATGCCTAAAACACAAATGTGAAGCCGTTAT
AGAGCAGAGGGCATACTGATTAGTATCCACGATTTTATCTAAGGTTTTCACTCAACGTGATGTTTTTGAC
ATTCATTCAAGTTGTAGTGTATACCAGTAGTTCATTCCTTTTTATTGCTGAGTAGTATTTCTTTGTCTGA
ATATACCATAGTTGATTCATTTTCCTATTGATGGACCCTTGGACTCCTGGAATCAGAAATGTTCCAATAA
GCACTTCCCTTGAGCATCACATAAGTGCTCGAAAGTTTAGGATTTTGTACCATTTCTAACTTTTGGATTT
GAGATGCTCAATCTGTACATTATTGGTGGGAAGTGTAAAATGGTAAAAATCCCTTTGACAAAAGGCCTGG
CAGCTTCTTAAAAAACGAAATATTTACTTATTCTATTACACAGCAATTCTGTCCTATTTACCCAGGAAAA
ATGAAAAAATATGTCCATAAAAAAGCCTGTATATTTTTAGTAGCTGTTTGTCATCTTGGTTATCATTAGT
AAAGCTACTAAAAATATACAGGGATTTTTACCATTTTACACTTTCCACCAATAATATACAGATTGAGCAT
CCCAAATCCAAAAGTTAGAAATGGTACAAAATCGTAAACATTTCTGATTTCTGGGTTTCGGTTGCTCAAC
CAGTAAATATAAATATTCCAAAATCTGAAATACTTCTGATTCTAAGCACTTCAGATAAGGGATACTCAAT
CTGTGTAAGCCTTCTGGTTATTCCATATTTTGCCAACATTTAGTGTTGGCAGTCATTTGATTTTTAGCCA
TGCGGTAGGTTTATAGTGGGAGCACATTGTAATTTGTACAGAGTGTTTGTAATTTGTATTTCCCTGATGA
CTAATGACATCGAGCACTTTTTCTTTCATGGTCTCTTTGTTTGTTTGTTTGTTTGTTGGTGTGTGTGTGT
GTATTTAATATTTTTTTCTAAAAAGTTATTGTTTTAGCTTTTATGTTTAGCTCTATGACCTATTACAAAT
TAATTTTTCTTAATGCTCTGAGGTATGGACTAGGGCTGGTTTTTTTCCATATAGATATCTAGGTGTTCTA
GCACCATTTGTTGAACAGATTTTCCTTTCCCACAAAAGCTTTGGCATCTTGATAAAAAATCAAATGATCG
TATTTTTGTGAGTCTCTATTCTGTTTCATTATCTACTTGTTGATACCTTTCCTACTGTGTCTTGATTACT
GTAGCTGTACAGCAACTTTTGAAGCTAGTATTAAGACTACAATTTTAGTTGTCTTTTTCAAGGTTATTTA
GCTGTTCCATGTCCTTTGCATTTCCATATGGACTTTAGACTCAGCTTGTTAGTTTCTACAAAAAAAAAAA
AAAAAAAAAGGCCTAGAGTGGTTAAGATTAGGATTGCACTGACTCTATAGACCAACTTGAGGACAGTAAA
CATCTTTACAGTGTTGAGCATTCTATTCCATAAACCTAGAATGTCTCTATTTAGGTCTTCTTTAATTTCT
CTCAGCAACATTTTGTGGTTTTTCAGTAAGGGAACTTACCTATCTTTTGTTAAATTTATTCCTATTTTGT
GATGCTATTGTAAATAAAAATTTCTAGATTTACATTTAGATTTTTTTCTACAATTTGCTGCCAATCCAAA
AGAATATAAATTTTGTATATTATTATTTATTTATTTTGAGGCAAGGTCTCACTCTGTTGCCCAGGCTGGA
GTGCAGTGGCGTGATCTTGGCTTACTGCAACCTCTGCCTCCCAGGCCCAGGAGATCCACCTCAGCCTCCC
AAGTAGCTAGGACTACAGCCATGTGCCACCATGCCCAGCTAATTTTTTTTTTGGCAAGGGGAAGGGGTAG
AGATAGGGTTTCACCAGGTTGCCCAGGCTGGTCTCAAGCAATCCACCTGCCTCGGCCTCCCAAAGTACTG
GGATTACAGGTGTGAGCCACTGTGCCTTGCCTGTCAGTCTTTTATACCCTATGATCTTAATAAAATTCAC
TTAGTACAATTAGTACCTTAGGCTTTCCTACATAAGCAATCATTTTATCTGCAAAGAGATTTGTAGATTT
TCCCTTCTTTATGCCTTTTATTTCTTGTTGTCTTATGTGGCTAGGACCTCAAGTATGCTGCTGAATAGAA
GTGGTGAGATTGGGCCTCCTTGTCTTTATTCCCTAGCCTAGGTGGAAAGTGTTCAATATTTCACCAGCTA
TAGGGTCTTCATAGATTTTTTTTTCCAGATTGAGGAAGAGTCCTTTTATTCCTAGTTTACTAAGATTTCT
TTTTCATCATTAATTGGGGTTAACTTTTATAAAATGTCTTTTTCTACTTCTGTTGGAGTTGTTATAAGGT
TTTTCTCTTTCCTTCTATTACTATGGGGAATTACATTTTCAAATGTTAAGTTTCATGTATCAGTCCCACT
TGATCACGCCATATTAATCTTTGTATATATTGCTAGATTTAGTTGAGTAAAAATTTTTAAAATTGTTTTC
AGGGTGGGATATTGGTATGTAATTCTCATTGTTTTGTAATGTTTTTGTCATCTTTTGGTATTAGCGTTTT
GCTGGCATCACAAACAAGTTGGGAAGTTCTCCCTTGATTTTCTCGAAGAGTTTGTGTAAGATGCCTCTTT
CTTCCTTGAATGTTTGAAGGTCTGGAGTTTTCTTTTTTTTTTTTTTGAGACACGGTCTGGCTCTGTTGCC
CACACTGGAGTGCAATGGCTTGATCTCAGCTCACTGCAATCTCCACTTCCTGTGCTCAAATGATCCTTCC
ACCTCAGCCTCCTGGGTAGCTGGGACTACAAGCGCACGCCACCAGTCTTAGCTAATTTTTGTATTTTTGG
TAGAGACGGGGTTTCACCATGCTGGGCTTAAGTGATCCCCACGGCTTCCCAAAGTCCTAGGATTATAGGC
ATGAGCCACCTGGACTGGAGTTTTCTTTTTTTGTATTTTTATTTTTTAGATGGAGTCTTGCTCTGTCGCC
CAGCCTGGAGTGCAGTGGGGCAATATCAGCTCACTGCAACCTCCACCTCCTAGGTTCAAGTGATTCTCCT
GCCTCAGTCTCCCAAGTAGCTGGGATTACAGGCATGAGCCAACACACCTGGCTAATTTTTGTATTTTTAG
TAGAAACGGGGTTTTAACCATGTTGTCCAGGCTGACCTGAGGCTGACCTCAGGTGATCCGCCCACCTTGG
CCTCCCAAAGTGCTGAGATTACAGGCGTGAGCCACTGCACCCGGCCTGGAGTTTTCTTTATATGTGGTTT
TTTGTAACGAATTTTCTTTACTAGATACAGCGCTTTTCCTATTTTCTGTTTCATTTTATGTCAGTTTTTA
ATAACTGGTGTTTTTCAAATAATATATCCATTTCATCTTATCAAATTTGTTGGCATAAAGTTCTTCATAA
TATTCTTATTATCCTTTTAGTGTAATAAGTTTATTCCTGGCTGGGCACGGTAGCACACGCCTGTAATCTC
AACAGTTTAGGAGGCTGAGGTGGGCAGATCACTTGAGGTCAGGGGTTCGAGACCAGCCTGGCCAACATAG
CGAAACCCTGTCTCTACTAAAAATACAAAAAAATTAGCGGGGTGTGGTGGCGTGTGCCTGTAGTCCCAGC
CACTCGGGAGGCTGAGGCAGGAGAATCGCTTGAACCTGGGAGGCAGAGGTTGCAGTGAGACGAGATCACG
CCACTGCACTCCAGCTTGGGCAACAGAGCAAGACTCCGTTTCAAAATAAATAAATAAATAAATAAATAAA
TAAGTTTATTCCTGATATTGTCAACTTGTGTTCATTTATAACTGGCTTATTTAGGAGTCTGTTGTTTAGT
TCCTAAATATAAGAGGATTTTTTTTTCTATCTTTTTAATTTGTGATTGCCCATTGAATTCCATTGTAATC
ACAGAAAATACTCTATAATTTTAATTCTTTTAAAGTTATTGAGACTTGTTTTATCACCCAGCTTCATCTA
TCTTAATGAACATATCATATATGTGAAAGATTTTATATTCTGCAGCTGTTGGGTGTTGTGTTCTAAAAAT
ATCAAATAGGTTAATATGGTTGATAATGTTGATCAGATTTTCTGTGTATGTGGGTTTTTTTGTTTGTTTA
GTTATATCAATTACTGATACTAAAATCTCCAGCTGTTTGTGGAATTGTCTGTTTTTTCTCTTTAATTTTG
TCAGCTTTTACCCCATTTATTTCAAACTTTGTTGCTAGTTGTTTACCCATATATAATTTCTTTGATTTTC
ACATATTGATCCTTTGTTATAACACGTATCTCTGGGCTTACTCCGCCTTGAATGTACATTATCTGAGGTT
AATATAGCCTTTTCCTGTTTACTCTGTGTCATGATATCTCTTTGCATGGTTTTTAATTTTAAACTTGTCT
GTGTCATATATTTAAAATGGCTTTCTTGTAGATAGGATATAGTTGGGCCTTGCTTTTTTTTTCTTTGGAG
TGTTTAGTCCATCATGTAATTATTGATATGATTGGATTTAGGGATGGGGTTTTTTGCTGTTTGTTTTCTA
TTTGTTCTTTGTCCTTGTTCTTCTTTGTCTTTCTTGGCTTTTTTTGTATTAGTTTAATATTTTTAGAATT
TTATTTTATCCATTGGCCTTCTAAAGCAAAAATATATTTACCTTTTGGAGGGAGAAGTGTTTATATGTAG
ATTACAAATTACTTCCTGAGGCCGGGCATGGTGGCTCACGCCTGTAATCCCAGCACTTTGGGAGGCCAAG
GTGGGCAGATCACCTGAGGTCAGGATTTCGAGACCAGCCTGGCCAATATGGTGAAACCGTGTCTCTACTA
AAAATACAAAAATTAGCTGGGTGTCGTGGCGCACACCTGTAATCCCAGCTGCTTGGGAGGCTGAGGCAGG
AGAATCACTTGAACCTGTGAGGCGGAGGCTGCAGTGAGCCAAGATCGTGCCTCTGCACTCCAGCCTAGGT
GACAAGAGCGAAACTCTGTCTCAAAAAAAAAAAAAAAAAAAATGAAAAAAGGTAGATCTCTTGCATCCAA
GTTGGTTCATTTGCTTCCCCTGTCCTTTATACCAGGGGTCAGTAAACTATAGCCACCTGGCCAAACCTGG
CCCACTGCCTATTTTATAAATAAAGGTTTGTTGGAAAACAGCCATACTCATTCATTTATGTATTTTCTAT
GGCTGCTTTTGCACTATAATGGCAGAGTTAAGTAGTTTCAGCACAAATCATCTATCCCACAAAATCTAAA
ATGTTTCTGTCTGAGCCTTTACAGAAAGTGTGCTGACCTCTGCTTTATGCAGTAGTTGTCATATATTTTA
CATCTACATCTTATAGCCCCAAATAAGATTTTACTTTAAATAGGTTTTGCTTTTAACAATTCTGTGCATT
TTGAGGAAACTAAAATAAAAAATGGTATTTTATAATTACCCTCATGTATACCAGTTTGAGTGTCAGTTTT
CCCTGAAGATCTGAGTTTCCATCTGATAACATTTTCCTTCAGCCTGAAGAACTAACTTTAGTTTGGAGTG
CAGGTTGATTGGGGATCAATTCTAGCTTTCTGTTGTATGAAATGTCTATTCTACCTTCATTGTTGAAGGA
CAGTTTTGCAGGATACAGAATTATAGATTGACAGGTTTTTTTCTTCTATTAAAGACTTCACTGTCTGCTG
GCCTCCATTGTTTCTGTTGAGAAATCAGTGATGGTCATTTGAATTGTTCCCTTGTATTCAATGTGTCATT
TTTGAGACCAGCCACCAGCCTGGGCAACACAGCAAGACCCTTATCTCCCCCTAAAAAAGAAACTCTAGGT
TCTATTGTTCCTTTGCTCCTCCAAAGAGTACTGTGTTGTTTAACAGGCAGTTTACTTCATACACTGTGTT
TGCGTCCCCAGCCCCCCATTTGCTTGGTTCGTTTATTCTCCAGGCTCTATGGTTGCTTTTAATATATTTT
GGTCAGAATTTGTAGCTGTTATCCATGGAAGAATTGAATCAAAAGGCACTTACTTGGTATTTTGTGGTGA
TTCATTTTCAGGTTGCCCAGTCTGCCATATTGCTAGAAAATTAAGTTTTATTACACTTATAATAAGGAGG
AAAAGATCCATTTCTATGTTGGGAAATGGTTGTGGGGGGAAAATGCAAGGGTTTTGGAGTCAGATCAGTT
ATGAAATTTGGCTTTGCTACCTGCTAGTTATGTGACATTGGTTGTTTTCATTTAACCTTTTTTATTTTAT
CTCAAAAATGGGGATTAAAAACACCGATATGAAATGTTTGTAAGATCTGAGCTAGTCCCACTCTACCTGA
ACCATACACGTATTTCTTTGATAAAAGGTGAGACTTTGTCCCACAGGGTCCATTTTGTTTAATTTGCTGG
AACCTAGGAATGAATATGCGTTTTCCTTTAAGTTTGTCAGATTCATCCCTGAGAGGTCTGAGATTTAAAG
AAAAAATCTATTACCAGATCACGTAAATGTTTGAAATCATTTTAAAAAATCACTTATTTTTCCTTTTATA
GGAATCACCTCCTCATGTAGAAAATTACTGTCTACCAGAAGCTATTTCATTTATTCAGAATAATAAGGAA
CTTGGGACTGGATTATCACAAAAGAGGAAAGCTCCTACAGAAAAAAATAAAATCAAACGACCTAGAGTAC
ACTAATCGCATCTACCCTTTAGTTACCAAACATTAAATGTTTTTAAAAATTGAAAGCCTGAATGTGACTG
TGATAGATTTGGGTAGTAATTTAAAGATGAGTACCTGAAGAATTCTGCTTCAGAGTATAATGATGACCCT
TCTTGAGTTTTGAACACCTGAAATTGTAATCACTGAAATATTAACTGTTTCTTAATAAAAAGTTACCTGA
AATAACAACAAAATACAACTCCTCAGCTAGCTTGCTGTTAAACCACATTGAAGTCTGTTAAAAGATATTT
ATTTTTCTTGTAAATATCTGAAGCTGTAGCTTAGTGGAAATTTTAGCAAGGTAATGGATTTTGCTTTAAA
ATGTCTGCCTTACAAATTCATAACAACAAGATTTGTCAGTCAGCATTTATTCATGTTTTCCCTGATTTTT
ATCTTCTCACCATTTTACCTCTTTTAACAGGAGCCTGAGCACAAGGTTTAATGAGGAAGCTGGGGCTATA
AATATGTGTGTATATATGTATATGTATGTTTGTACAAATCTCCATGATGTTTGCCAAGTTTGAATGCGCA
AAACTTGGAAAATGTGACAATAAAGAATAAAAGTAGTAACTCAAATTAGTATTAAGATGTGTTTACATAG
ATAAATTTTTTAAAAGAGCACCCTGGCATCTGTTCTTCTGTTGAAGCAACAAAACATTTATGTAACGTAA
AAAATGAATTGCAGGGGCCTGGGTGCGGTGGCTCGTGCCTGTAATCCCAGCACTTTGGGAGGCTGAGGCA
GGCGGATCGTCTGAGGTCGGGAGTTTGAGACCAGCCTGACCAACATGGAGAAACCTCATCTGTACTAAAA
ATACAAAAAATTAGCCCAGTGTGGTGGCACATGCCTGTAATCCCAGCTACTCAGGAGGCTGAGGCAGGAG
AATCGCTTGAACCCGGGAGGCGGAGGTTGCGGTGAGCTGAGATCGCGCCATTGCACTCCAGCCTGGGCAA
CAAGAGCGAAACTCCATCTCAAAAAAAAAAAAAAAAAGAATTGCAGGCCAGGCACGGTGGCTCATGCTTG
TAATCCCACCACTTTGGGAGGCCAAGGTGGGTGGACTGCTTGAACCCAGAAGTTCAAGACCAGCCTGGAC
AACATGGCGAAACCCCATCTCTGCAAAAAATAGCAAAAAATTAGCCGGGCATGCTGTTTGATGCTTGTAG
TCCCAGCTACTTGGGAGGCTGAGGCATGAGAATTACTTGAACTCAGGAGGCAGAGGTTGCAGTGAGCTGA
AATCATGCCACTGCAACTCCAGCCTGGGTGACAGAATGAGACCCTGTCTCAAAAAAAATAAATGAATTGC
TTAACATTTCTGATATTTGTTATTTAGAATATTGCCCTATTAGATTAAAACATACTGTTATTGGAACTCT
GTATTGCATTAGCCTTTATTATGCTAAACCTAAACACCTAATACTACATGCGTTACCAGAATTCTCTATA
AATACACCTAATTTCATTTTAAGGACAAACTAGAATCCGTTAGGAATGTAATTTTGATGTAAACCGTTCA
ATTCCAAATGACTTTTTGAAAGGCAGATTCAAACAGTGAAGGAGAGAGTGCTCAACTTGGACACAAAATC
CCTTAAATTTTCATCAGATTTTCTTCAAAATGGAGTTGAGGGTATCCCTGCTGCTAGAGGATTGGTCCTA
CAGGGTTTTCAGCTTTGAGCTAGTTCACAGCTTAGGACAATACAAATTCTACACAGAGAAATAGGTAGCA
TTATGATGTAAACTGTCCCCCTCCCACCAAACAAAAACCACATGGGCCTGTCTTTATCAGAGCTATTTTA
TTTTAGCTGAATCATTTTGTAGATTGTAGTACGTTTCCCTGAATTTTATTACTCAGAATCCTTTGAAATA
GGCCAGGCACAGTGGCTCACACTTGTAATCCCAGCACTTTGGGAGGCCAAGGTGGGCAGATTACTTTAGG
TCAGGAGTTCGAGACCAGCCTGGCCAACATGACGAAACTCCATCTCTACTAAAAATACCAAAATTAGCCA
GGTGTGCCGGCACGTGCCTGTAATCCCAGCTACATGGGAGGCTGAGGCAGGAGAATCACTTGAACCAGGA
GGTGGAGGTTGCAGTGAGCCGAGATCGCACCACTGCACTCTAGCCTGGGTGACAGAGTGAGACTCCATCT
CAAAAAAAAAAAAAAAAAAAATTCTTTGAAATAGTTACTTTTTAAATTACCTAATATACCACTTTCTTTT
CTGCCAAAATTATGAATGGTTTTCCAGTTCAAACTTAGACACACAGTTTACCATTTAAGGCTCCAGGTCT
CAGTTTCTCCTCCTAACTTTCCAGCATCTGCCCATACTCTGTATAAGCCACTTGAACTCTGGCTTTGGCT
GTTAATCTGTAAATGTCCTAATGCCAGCAACAATATCCTCTTATATACAAATAATTCCAAGCATAGGTAG
TCTTCTTGTTTAAAAAGGGGAGGGGGCAGAACTTGCCTTGATTAGATTAGTATGAGTTTCTTTTCTGAAT
GGCTACGATATTTTAATCTGTGATATTTAAATTATCACCTTATATCCCTCCACGAATTTTCCTGTGCATT
AGTCTGATCTCACCAACATTCGTTAATTCAACAGGTAGGAATCCTCAACCTGTGCCTAGGATTCAGCAAC
AAACCACCTGGAACACATGGTCCCTTAGCACTTGAAGAGGTAGGAATCCTTACTGTAAATACAGTACTCA
ATTATACTCAGATTTAGTATTTACACTGTTAAGGACTGAGCTTGTCCCCCTAAAATTCACAGGTTAAAGC
CTAACCTGCTATGTGACTGTAGACAGAGCCTTTATAAGGGGATTAAATGAGGTCATAAGAATGAGTCCCT
AACCCACCAGGACTAGTGTCCTGGTAAGAAGAGGGAGGCACACCAGGAGTTTGCCGGCACGGAGGAAAAG
CCCTGTGAGGACACGGGGAGGGCAGCTATCTGTTTACACACCAGGAAGAAATGTCTCACCAGATACCAAC
CCTGGCCGTCTCCTGGATTTTGGATTTCCAATCTCCAGAACTGTGAGAAAATAAATTTCTGTTTAAGCCA
CTCAGCTTGTGATGTTCCTTTGTGGCAGCCTGAGCAGACCAGTAACAATATTAACAGTAATAAGGTTCTT
ACTTTGGGGCCTGAGATGCCTGGGAATGGAACAGGCTGTTTTGTATATGAGCATTTTTAGGGTAGAGTTC
ATAATTCTAGAGTTTGGATCTGTGACTTTCCACCCAAAAGTCACAAACCACTGCTCTAATGCAAACCTAT
TATTTCACATTTACACCTCTCACCTAGAAGTGCTTTAATACAGGCTGCTTTGTATCAGGGCTTGGTTTCC
TTCTGACCTTTCAAGACATAAAAATCATTACTAGCTTGAGGCCATGCAAAAATAAGCAGTGGGCCATAGT
TGACTGTTCCTTATTTTCTATCCCTAAAAAGATTTTAAAATCTAGATCTGTCAACAAGCAAGGCACCCAG
CTCAATTTTTTTTTTCTTTTGAGACGGAGCCTCGCTCTTGTTGCCCAGGCTGGAGTACAGTGGCATGATC
TCAGCTCACTGCAACCTCTGCCTCCTGGGTTCAAGCCATGCTCCTGCCTCAGCCTCCTGAGTAGCTGGGA
TTACAGACGTGTGCCACCACGCCCAGCTAACTTTTCTATTTTTGTAGAGATGGGGTTTCGCCATGTTGGC
CAGGCTGGTCTCAAACTTCTGACCTCAAGTGATCTGCCCGCCTCAGCCTCTCAAAGTGCTGGGATTACAG
GTGTGAGCCACCGCGCCCTGCCACTCAGCTCCTTTTCTACCATCCTGAGTAGCAGTCTATGGCAGACATA
CTTGACAACAACTCAGGCATACCCTGGGAAGTGACCCTGTGGTCTAAGAAGAATGTATGTTTGGAGTTCC
AAGTTAAGGAATCCAGGAGTGGCCAGTCCAGAGACTGACTCCTTATCATCAATGACGGATGTCCATACCC
CTGCCCCATTCCTTGGAATGCAGGCTGTATAGGGGATGGTGGCCCTTTGTATTGGGTTAAGTGGAAGTTG
TTAGGTAGAGGTTGCTATGTGAAAATGACATGTTAGACTGCATGCTTTTAAAAAACAGTAGCAATTTTTG
GCCGGGTGCGGTGGCTCATGCCTGTAGTCCCAGCACTTTGGGAGGCCGAGGCGGGCGGATCACGAGGTCA
GGAGATCGAGACCATCCTGGCTAACACGGTGAAACCCCGTCTCTACTAAGAATACACACACCCAAAAATT
AGCTGGGTGTGGCGGCGTGCACCTGTAGTCCCAGCTGCTGGGGAGGCTGAGGCAGGAGAATGGTGTGAAC
CTGGGAGGCAGAGCTTGCACTGAGCCGAGATCGCGCCACTGCACTCCCGCCTGGGCGACAGAGCGAGACT
CCGTCTCAAAAAAAAAAAAAAAAAAAAAAAAAAAAAAAAAAAAGGAGCAATTTTCCTGTCCAGCCCACTG
CTCCTGGACTGTTGTGTAATGTAAGTCCACAATAAACCCTATGTCTCATTCACTGGCTCCACATCTCTTC
TTCAGCCTCCCTCACGTGGTACCATCCCCACTAGAGTCAATAGCAGTCTGGCATGACAAGTAGTAGCATT
AAGCAGAGCCATCTCATGCTTGTTTAACATTTGGAAATCTGCAATGAAAATTATCAAGTTTTGGCTGGGC
GTGGTGGCTCACACCTGTAATCCTAGCACTTTGGGAGGCCGAGGCCGAGGTCAGGAGTTCGAGACCAGCC
TGGCCAACGTGGTGAACTCTCATCTCTGGGAGAAAAATACAAAAATTAGCTGGGAGTGGTGGTGGGCGTC
TGTAGTCCCAGCTACTCAGGAGGCTGAGACAGGAGAATCGCTTGAACCCGGGAGGTGGCGGTTGCAGTGA
GCCGAGATTGTGCCACTGCACTCCAGCCTGGGCGACAGAGGGAGACTCCGTCTCAGAAAAAAAAAAAAAA
GAACTTTCTGCTGCTTTGAGGGGAGACTGAAGAAGTCAGGCTCTAATGCATCCCTGCTGACCTTGGGATG
GGAACAGGGCAAACAGCATTTTCAAGTCATTTCGAAGGAGCATTTGGTTATCATGTTCCTTCTCTACCCT
TACTTAAATCTAAGGTGAAGAGTGGAAGCAAAGCCTCATGGTTAAAAACATGCTGGAGCCAAAACACCTG
GGTTGGAGTCCTGGCTCTTTCATTACTAGCTATGACTCCAGCAAGCTATTTAACCTTTCAGAACCTATTT
TCCCCTCTTTAAGGGGCTTTCAGGGTTAAACGAGTCAACATTTGTTACCTGTTGCCTGGCTCAAGGTTAA
AAACTATACAATTTTTTAAAATTCCAGTAAGTGATTTAACACTTTTGCCAGCTGGAGCTAAGGTTTTAAA
AAAAGAAGAGGATGTCAGGACAGAACAAAAGAATATAATGGGAGCTTGCAACCTCCTCTTCACTCCCATT
ACCATGGTGCCACTGAGGTTGGGAGTCCCTGACTTCCCTGTTTAATTCAGGTTCTAATTAAAGACATACA
TTTGAACTGTAAAATCAGAGAAATCCAATAAGGAAGCCATAGTAGTCCCAGCAATTTATAATAAAACCAA
AGACAAACCTAAGAGATTCCAGTCTGTGCCCACACCTCATTATGAAAAGATGCTTAAGTATAACCCACAT
GATCTTTAAAAGGCAGGAACTGGTGAGTAAGGTGAGTCACAGACCCAGAGTGTGTAGGTGGTACAAGCTA
CACTTGGCCTGAGGTTCTGATTCTTAAATAGATGAGCTATATGATTCTGCGGCTGCCAAAGCTTATCTCT
GGTAAGGTTAATTATGGTTGAATTCATAGTCACCAGGAAGAATAGCAGAATTCTAAAGAAACTCTTAAAA
CCCAGAGGTGCTATAACTGAGCAACATAATGAAAGGGCAAATTTAAAGTGGCCAGGGGTAGTGTAAGGCA
GATTAACATTTACAGTGAGTAAACTCAGGAATGGTCAGATCTGCTGTCAAAAGACAAGTCAAACATCATG
GAAGTATTGTTGGAGAAGGATAACGAGTAGCTACAGAAGTTACATTGATGATGAGGCAGTTATCTAAGAA
ATGACAGATAAGATATTAAAAATGGTCTTTATATACTGATAACTACTGAACTACAAAAATGTGTAGTTTT
GTAAAAAATCAAAGAAATTTACTATTTCAGTTGACAAGTCTTTCCCTCCAACTTCAGTATCCACTTTCCC
TCATTTAACAGTATCCAAAAATGCATTTAGGAATATGATAATCATTTCTGGTCTAAGCATGCAATTTAAG
GGATTTCCTGCTTGAGCTAGCCCTATGGTTGTCTTCCAACTGCCTACTAGGGAAATAATTTATCATTTTT
TGACATAACCCAGTGGTTCCCAGATTTTGCTGCACATTAGAACTATGGGACTTGAAAAAACATTCCTCTG
CTCGGGTCACACTCCATACTAATTAATTCAGAATGTCTGGATGTGGGAGTCGGGCATCACTTTTTTTTTT
TTTGAGACGGAGTCTTGATCTGTAGCCCAGGCTGGAGTGCAATGGTACCATCTCGGCTCACTGCAAGCTC
CGCCTCCCGGGTTCGAGTGATTCTCCTGCCTCAGCCTCCTGAGTAGCTGGGACTACAGGCGTCCACCACC
GTGCCCGGCTAATTTTTGTATTTTTAGTAGAGATGGGGTTTCACTGTGTTGGCCAGGCTGGTCTTGAACT
CCTGACCTCAGGTGATCCGCCCACCTCGGCCTCCTAAAGTGCAGGGATTACAGGCGTGAGCCACCGCACC
CAGCGAGTTTTTTTTTTTTTTTTTTTTTTTTAATGATTCCCAGGTTATTCCAAAATGCAGCAGTTTGAGA
ACCATGGACATAATCTTACAGAGTTGCTCCACTTCTTATGATTAAAATTGCGAAGGCTGGGTGCTACTCT
CCATCATATTTGGAGATGAACCATTTTACAGAGGTTTCTTGGGGCATGCTTTGACTGGATCCCTGAACAG
TTAGGTTGAGGGATTAAAAAGACTTTTAAAGTGGATGACTTAAATCCTACCCATCGTCTGTATTACTATA
TCCAATTATTTAGAATTCTAGTTCCCACTTGGGCTGCAAATTCAAAAGAATTGAACTGCCTTAATGATTA
CTTTTATGTATATCCTAAATTCCTGATGTGAACATAATTCCTATTTTAGATAATACAAATAAGATTATTT
AAAGCAATTATTTCTAAGGCCAGGTGTGATGGCTCACACCTGTAATCCCAGCAATTTGGGAGGCCGAGGT
GGGCAGATTACTTGAGGTCAGAAGTTCAAGACCAGCCTGGACAACATGGGGAAACCCTGTCTCCACTAAT
ACAAAAAAAAAAAAAAAAAATTAGCCGGGCGTGGTGGCATGCGCCTGTAATCCCAGCTACTGGGGAGCCT
GAGGCATAAGAATTGTTTGAACCTGGGAGGCAGAGGTTGTGGTGAGCTGGGATCGTGTCACTGCACTCCA
GCCTGGGTGACAGAGTGAAACTGTGTCAAAAAGATAAAATAAGCAATTATTTCTTACTCAATATATCACT
CATAATATTCATTATCTTTATTTCAAGCCCAGGAGAAGCAATTCATAACTTGCCTGCATTCTGCAGGCCC
CATTAGTACCTAGTTGTAGTAACTGGTGAATTAAAACCATTACAAACCTGCAGTTTCAGGATGCTAGTTA
TTAGAACACCTGTCCATATGACTTTCTAACTTCTTGGCTCTCCTTCTGATCTGACTACCTCCATTAATTT
GTTTGCACTCCCTAACTTGAAATCGTGACCTCATTTGTTCCTTTGTCAGTCTAGACACCCATAGCATGAC
CTGTCCTAAATTCCTACCCCACACTCTTAACCTCCTGACATCTCCACCTACGTGTACCTACCCCAGACCA
ATTTCACATTCTAACAGAGGGAGATAAACAACCAAAATGTTTAGTGTGCTAAATAAAGAGTAAGAACTAC
AAGCCAGGCATGGTGGTTCACACCTGTAATCCCAGCACTTCGCGAGGCTGAGGTAGGAGGATAACCTGAG
CTCAGGAGTTTTGAGATTAGCTTGGGCAACATAGCAAGACCTCATCTCTACTAAAAATAAAAAAAGTTAG
CTGGGTGTGGTGGCACGCATTTTAGTCCCAGCTACTTGAGAGGCTGAGGCGGGGAGGATCATTTGAGCCC
AGGCCAAGGCTGCGGTGAACTATGATCACACTACTGCACTCCAGCCTGGGTGATGCTGTCTCAAAAACAA
CCACCAAAAAATCCCCCAAAAACTATGAGTCTTGGTGCTGAGCAACTGGTAGTCACATGCAGAAGAATGA
AGCTGGAGCCCTAAGTCACAATAAAAAAAATTAAAATAGACCAAAGACCTAAATGAAAGAGCTAAAAACA
TAAAACTCTTAGAAGAAAATAGGCATAAATCTTTATGACTTGGGTTAGGCAATGTTTTCTTAGATATAAC
ACCCAAGCACAAGCAACTAAAGAATAGAAAAGGTGGACTTCATCAAAATTGAAAATTTTGTACTCCAAAA
GACACTTCTAAGAAAGGGAAAAATGGTAGGAGAAAATGTTTACAATTCATGTATCTGACAAAGAAGTAGC
ACCTGAAATTTAAAACACTTGCAACTCAACAATAAAATTATAAGTAAACCAATTTAGAAATTGGCAGAGG
ATTTGAATACACATTTTTCCAAAGACATACAGATGGCCAGTAAGCACATTTAAAGATGCTCAACATCACT
AGTCATTATAGGAATGCAAAATAAAACCATAATGAGATACTTCACACCCACTCGGAAGACTAGAACCAAT
CTGAAACAAGTGATCACAAGGATATTGGAGAAACTGGAATCCTCTGACACTGCTGGTGGGAATAGTAATA
TATAATATGGTGAGGCTACTTTAGAAGAGTTTGGCAGTTCCTCAAAGTGATACAGCCTTTCATCTCTAAG
TATATACCCAAGAGAATTGGAAATGTTGATACAAAAACTTGTACATGAAAGTTCATAGCAACATCATTCA
TAATAATGAAACAACCCCAATGTCCAACAGATGAATGTACAATTTAAACGTGGTATACCCATACAATTAC
CTATATTGTTTAGCAATAAAAACGAAGCACTGATACATGCTAAGCCTCAAAAACATGCTCAAAAACAAGT
GTAGAAGCCAGTCTCTGAAAAACAAATTGTATAACCCTGTTTATATGAAATGACCAGAATGAGAAAATCT
AGGCCGGGCACGGTGGCTCATGCAGCTGTAATCCCAGCACTTTGGGAGGCCAAAGCGGGTGGATCACCTG
AGGTCTGGAGTTTGAGACCAGCCTGGCCAATATGGTGAAACCCCATCTCTATTAAAAATTCAAAAATTAG
CCAGTCGTGGTGGTGGGTGCCTGTAATCCCAGCTACTCGGGGAGGCTGAGGCAGGAGAATCACTTGAATC
CGGGAGGCGGAGGGTGCATTGAGCTGACAATGTGCCACTGCACTCCAGCCTGGGTGACAGCAAGACTGTC
TCAAAAAAAAAAAAAAAAAAAAAACAGAAAAAAGAAACTAGAGACAATACAATAGTGGCTGCCTAGGACT
GAGGAATTTGGGAGGAAAGAAGAAAATGATGACTAATAACAATAGAATTTCTTTTTGGGGTGATGAAAAT
GTTCAAGAATTTTAAGAGTGGTGATGGTTGCATAACTTTGAGAATATAAGAAATTCTTAAATTGTACACT
TCAAAAGGTGACTTTTGGCCAGGTGTAGTGGCTCCCAAAGTGGCTGTAAAACATTCCAGCACTTTGGGAC
GCAGAGGAGGACTGCTTGAGACCAGGAAGCTTGAGAACAGCCTGGGCA

An exemplary DNA sequence is human Cdv3 provided below.

gi|224589815: 133289930-133311620 Homo sapiens chromosome 3,
GRCh37.p13 Primary Assembly
(SEQ ID NO: 311)
AAAGAGGAAGGGACCAGCTTCACAGAGGAGAGAATTACTACAAACAATGACACAACCACAAAGACACTGG
TCTCTCTACTGGCCTGACCTGAATTTCTTACACATGATCAAGTTCTGGGATCATGCCATCAATCACACTG
AAAAATGTAGTGGGAAATTCTGCAGCGTCAGCCCTCAACTACTGTTCCTTCTGTGTATTTTCTCAAGTCA
TTAATTCTTTCAAGACAATGATGAATTGATAAGGACTTAAGAAAGATATAAATAACTGAAACTTATTTAT
ATCTAGATCTTCTCTTCTAGATTATAAGGGAATTGAATGCAGGAATCACAAATTACTCACTTTTGAATAA
AGCACTTTGCCCGAAATATAGCAAAATAAGTGCTTCATAGATGAGGAAAGAAAGAGAGGGAGAGGGCCGG
GCATGGTGGCTCACACCTGTAATTCCAGCACTTTGGGAGGCTGAGGTGGGTGGATCACGAGGGTCAGGAG
TTTGAGACCAGCCTGGCCAACATGGTGAAACCCCGTCTCTACTAAAAATACAAAAATTAGCTGGGTGTGG
TGGCGGGTGCCTGTAATCCCAGCTACTCAGGAGGCTAAGGCAGGAGAATCGCTTGATCCTGGGAGGCAGA
GGTTGCAGCGAGCTGACATGCCATTGCACTCCAGCCTGGTGACAAGAGCAAGACTTTGCCTCAAAAAATA
AGTAAGTAAATAAATAAATAAATAAATGAAAGAGAGGGAGGGAGGAAGGAAGAAAGAGGGGGGAGGAAGA
AAAGAAGGAAAGAAAAGAGGAAGGGAGAAAAAAGAAGGAAAGAAAAAAAAGAAAGAAAATAAATTTGATT
AGCGATGGTAAGGGAGGGTGATGGTGATGGGCAGTACTGAACCTGGCCTGCATCACAGGAAGATATATGT
TGTCATAGAAGGAAGTCACCTTGGGAGCTGTTCATGTTTGGGGAATAGTATATGTATAAGCTCAAGCTAC
TTGCTGGCCAAGGCTAGGTCCTTGATTTCTTTTGACATTCAGAAGTTAGACTTTTTTTTTTTTTTTGAGA
CGGAGTTTCACTGTTGTTGCCCAGGCTGGATTGCAATGAGGCGATCTCGGCTTACTGCAACCTCCGCACC
CCCGGGTTCAAGCAATTCTCCAGCCTCAACCTGTAGCTGGGATTACAGGCGTTAGCCACTACGCCCGGCT
AATTTTCGTGTTTTTAGTAGAGACGGGGTTTCATTATTTTGGTGAGGCTGGTCTCGAACTCCTGACCTCA
GGTGATGCGCCTGCCTCGGCCTCCTGAAGTGCTGGGATTACAGGCGTGAGCCACCGCGCCCAGCCAGAAG
TTAATTCTTTAATAGCAGTGTAAGTAGGGGGTAGGAAAAGAGAAACTTTAATGGGGTAACTGTAAAGGAA
AAGACGAAGAAGTCTCTCCATTTACTGGCTGGATTCTATCTACCCCATATGGCTACCAACATTTGTCTGA
GTCATTTCCTCAATGGTTGTGACAGCTCCCTCTTCTGCTTTTAGAAGGAGGAAGTAGAAAGTTTAAGAGC
TTACTTTAGACCACCTTTTGCATATAGAATTCATTTCATTTTCAAGGCCCCAAGGGTATTTAAATGGGCT
TTAAACCCATTTAAACCTGGCTTTCGCCTGGGCATTTTGGTCTCCACCCCTATGATAAGACAGTCCCCTC
CTTTTATCAGGGATAGAAGCTAAGGATAAACTGTATCATCATCTAGACTCAAGTCAGGGCGTTAAGCAAT
GTGCTGAGCAGCTAATAATGTTGACTCTCTTCCCTCCATCCCCCTATCTTGTAAGAAATAGTATACCCAC
TTTACAGATTAGGGACTTGACGATAGGATTTATTGAACTTGACCTTGGTAAACATAATCCAGAGGGTGGA
GTTCAGACAAAATCCCTTTCTACCTACCAACCAGCCTATGAAAGATCTTTTAGTAAATGGGACCTAAACT
TCAGGCTCACTGCTCCTCTCACTTTCACAATCAACCGGTAAAGAAGAAAAGTAATGGTTGCCACTCCAGA
GGGGGTGGAAGTACAGAAGCGATCAGAGGAGAGGAACAATCTTTAACACAGTTGGCTTAACGCAGTGCCT
GTCCGTTTCGCGACAGTTCAAACGACTACTATGGGGCCACGGTTTGTTCTGGGCCAGGAAACACACGTGC
ATCCTGCGCATGCGTCCAAAAGGAACCCACTGCCCCTCCTGCTGGACCTCTTTAGCCAAGTCATCAACCA
GCCCTCAACATGCCCGCCCTCAAGACTAAACCCTCGGCCAATCGGGAAACCGGAGTCGCCCGGCACGTCA
CTTATTTTGCATATGCCCTCCTCTCCCCGCCCCCATGCTTGGACTCCGCCCCGTCTGGGTCAGGGATTGT
CACCTAAGAGCTTCGTCCTGGCACCTGATTGGCTCTCTTTCCAGCTCCGCCTCCATCTCCTCCCGCCGCC
CGCCCCTTGGCGATCCCACAGCGGCAGGCAGAGCAAAATACGCAGGCGCAGGCGCTTCCTCTCTCTTCTA
GCGGCCCGCCCTCCCTCCTCCCTCTCCCTCCCCTCCCCACTCCCTTCCCCTCCTCTCCAGGCCTGCGCGC
GCGCCTCTTCCTCCGGCACGCGCCGCTGCTAGCCGAGCACTCTCGCCAGAACCTCTGGCTCGCGCGTGCC
TTTTCCCCTCAGGTTGTGGGGAGAGCGGAATCCTGCTCCGCCGTCGCAGCAGCAGCGGCAGCCCCGGCAG
CCTCGGGCGACAGCGGCGGCGCGCGAGCCCCCGGGCGGACCGTACCACCGCTCGCCAGCACGCAGGGGGA
GCCGCCCGTCTCGCCGCGCACGCCTCGGCGACCCCGCGGGGCTGAGGCGTCGCCGCGCCCGGCAGCGTGA
GCGCAGAGCCGGCCTCGACCCCGAGCTCGGAGCCCCGCGGGCCGCGCCCGCCGCCGGCCCCACCCATCCG
GGTCGAGGAGGCCGAGGCCATGGCTGAGACGGAGGAGCGGAGCCTGGACAACTTCTTTGCCAAGAGGGAC
AAGAAGAAGAAGAAGGAGCGGAGCAACCGGGCGGCGAGTGCCGCGGGCGCAGCGGGCAGCGCCGGCGGAA
GCAGTGGAGCCGCGGGTGCGGCGGGCGGCGGGGCGGGCGCGGGGACCCGGCCGGGTGACGGCGGGACCGC
CAGCGCGGGGGCTGCGGGCCCAGGGGCCGCCACCAAGGCTGTGACGAAGGTGAGGGGCCGGGAGGCCGGC
ACTCGGGGCCCGGGCCGCGCGCCGGCGCCCCATGTGCCCGCCCCCGCCTGCCGGGGAAGCGTCCGGGAGG
GGCCGCGGAGGCCGGGCGGACGGGCGCGGGCCGCCACGTGACGCAGGCTCTCCACCTCGGGCTGGGCTCG
CCAGGGCCGGGACCCCTTCCGATATCCGCGGTGGAGCCGCCCTTCCCACCCCGCGTCGCTCGGCGTTAGC
CAAGGCCCGGGCGGCGCCACCCTCCGGGGGCACTAGGTCTGGGGCCGCAGTGCCCAGCACAGAGCAGCGT
TTATCGGGTGACGTCTAGGCTGAGTAATTCCGTGTGCTGACCTCAGCCCAGTAGTTTGAGCTGAATTTTC
GCTTTCAGCAGCCGCATTTGTAGCAGCTCTTTTGTGATGACTTAGTCTCTAAGCCCGTGAAAGAAAAAGA
AAACTCTCGGTGGCAAGGTTGAAGTAGAAATGTAGTTGTGAAATTCCTATTGACTTCTTCCTTCGGCTTT
TCTTAGTTGCTTAAAGTAACAGCTCCCATGCAGGAACCCAGCGGAAGTATAGTGTTAGCCTACGAGCATG
TTTGTCCTCCATTTAGCCTAGTTTGCCCCAGAACGAAGCAAGGGCTTTGACCAGAAGACAAGTACATACT
TAGTTTAGGTTCCAGCCACTTGATCCACTTTTCGTAGTGTGTTATTTGCTTAGTTATATGTCTACAAATA
GCTTTAATTGATCAAATGGCGTTTGTTTTACAGGACGAAGATGAATGGAAAGAATTGGAGCAAAAAGAGG
TTGATTACAGCGGCCTCAGGGTTCAGGCAATGCAAATAAGGTATAGTCTGGTTACAAATGTGTTTAGATA
ACCTTTGGGATAGATATTGGATACAAGTTAGGTTTTCCTTTGGCCCCAAAGCAGCATCATAGTAGCCTTG
TGCTCTAGATATGCTTATTAGGACTCAAATGGGTTCTGTCTACTCTTTCTCACTCCATGGAGGCAGCCCT
TCCGTGTGCTCATGAAATTAGCTGCTGACTCTAAAATCCTGTGAGAGCTTGCCTACTTCCAGTTCTGGTT
AGGACTTTTCATTTGAGACAGCTAAAGTGGATTTTGTCGCTTCAAAATTTTTACTTTTAACATCGGTGAT
TGTACTTAGTTTTGGTTTCAAAAGTAATGTGGATGTTTTTCACATCCTCTCCAATAAGCAAGTGAATATA
TGTTTTTGTAATTGAAGTATATCATACTTACAGAGAGATAGAGGTTTTATGTGTTCACACAGTGCAACCA
ACCTGTGCAATAAGCACCAGGTCAAGAAACTGAGTATTACTGATACAGAACAAGGTTCCCCTCCTAACAC
ATACTCACTGCATTCACCTTCAACATAACCACTATTCTAACACCATAATTTTTTCTAAAACTTCATAGGA
ATGGAATTATATTTGTAAACATGTTATTTTTTAAAGTTCAAAAAAATAGTAATTTTTAAAAATTGGGTAG
TTTAGAAGATTTGAAATTAACATTTTGTCAAGTCCAGTCTATGGCAGTCAACTTTAAGGTTGATGTTAAG
TAAGTGCTATATTTTGGAGGGTTCATAAAGTAGGAAAAAACAAATGAAGATATGGTTCTGCTGTAAGATG
AAAAAATCCTTTGCTCCCGGTTTAAAGAAATAAAGGATCTTGATAAACTGTCTCCAGTTTGAAGGTTGCA
TTTTATAGGCAATGCCAGTGAGTGCGAGGCACAGTGGAGGAAATACTTTCCTTAACTGAAAACTGTCTTT
TGACTTCTCTGGATGTTAGTATCTATAGCTTTAGATAACTTACTCAAAAAGCAGGGTTAATATTATTGAC
ATTTGTTGAACACCTACCACAGCTAGGCATTAATGTCCTACTGTGAAAACATCATTTTGTGAGCTTAACC
AAAGTGAAACCCCCAGATTGATAATGGGAAACTTTCAGGAACGATGGCTTCTCCACTCTTGAGATTTCTT
TGTAGTGGTGTTGAGTAGAAGTGATTAGACTTGAGTTTGATTCCCAACCACTTGTTGGCTTCCTTAATGT
GTCTTTCTCTTTTCTGTCTCCTGGGGCTTGACCTGTAAATAATGGGGAGGAGGGAATGTGAGGTTCTTAC
CATTTGGTGTTGTGGAACTGCCTGTGTGTGTGGCTCCGGTTCTGTTGTAAAGTTTGGCTCTTCTTAAGTC
TTTTCTTTCCTAAGTGACTGCAGGTTCTTGAAAACGTGAGTGACTGGAAGAATAGTGTTTGGCCATGTTG
AACTTGGCAAAATAGCACTTTTCTGAAGATAAAATTTTGGTTTCAAGTTCACTGAGGATTAGAAATTGAT
TTTGTTAAACTATGGAGATTATTTAAACAGAACCTGTGTTTTTCTGTTACTGACTATAAATTACATGTTC
ATCTTCAGGGTTATTTTGATCTTGATCTATTTTGGTTACTTTTGAGATAGTGAAAAGTGTCTTAAATAAC
TTACATACTTCACCCTTGCCAGGGATATAAGTAAGTATCAGCTAGTACAAGCATTTAAGTTCTGTTCAAC
CTGAAGGTAGACTAGCTTTTTTTTTTTTTTTTTTTTTTTGAGACGAAGTTTCCCTCTTGTTGCCCAGGCT
GGAGTGCAGTGGCAAAATCTCGGCTCACTGCAACCTCCGCCTTCTGGTTTCAAGCGATTCTTCTGCCTCA
GCCTCCCGAGTAGCTGGGATTACAGGCGCCCGCCACCACACCTGGCTAATTCTTGTATTTTTAGTAGAGA
CGGAGTTTCATCATGTTGGTCAGGCTGGTTTTGAACTCCTGACCTCGTGATCCGCCCGCCTCAGCCTCCC
AAAGTGCTGGGATTACAGGTGTGAGCCACAGCACCCGGCCTAGACTAGCTTTTATATTGTTTTTTTTGTT
TTCTTAGCATCTAGTCATTGGCCCTAAGTGGCCATCCTAGTAGAGGTATTCCACTAACCTGCAACACTGA
GAAACACTCCTTCCCATATGTTCTTCACTCAGATCCACCAGTCTCTGCTACATGTGCTTTATCTTTTTCT
ATTTCTCGGTATACTGAGAGCTGCAGATGTCACATTCAGTCTTTTTTTCTTTTTTTTTTTTTGAGACAGA
GTTTCGCTCTTGTTGCACAGGCTGGAGTGCAATGGTGCGATCTCAGCTCACCGCAACCTCAGCCTCCCGG
GTTCAAGCGATTCTCCTGCCTCAGCCTTCCGAGTAGCTGGGATTACAGGCATGTGTCACCACGCCTGGCT
AATTTTGTGTTTTTAGTAGAGACGGGGTTTCTCCATGTTGTTCAGGCTGGTCTCAAACTCCCGACCTCGG
GTGATCCACCCACCTCGGCCTTCCAAAGTGCTGGGATCACAGGCATGAGCCTCTGAGCCCAGCCAGATGT
CACATACAATCTTAAAGTTCTTGACCCTGGTCCTTATAGTAGTTAGCATATTAGTTCTTTACCTGTACAT
TTGAAAGGGGAAATTTTTCATTAGCAGCAAACGTATTATATATTAAGATCCTATTTCACATTTACCAACC
AGCTCAAGATTTAAGTACCAAGGTACTTAATGTGCCCAGTTTTGTGTCTGAATTAACAGATCTTATTCAA
GGTCATGCTGTTATAGACAGATTCTTGAGAGGGAAGCCATTGAGGTGGTTATAAAGGAGGCTGTCATTTG
AGTTAGATCCTTGGTTGTTTGTAGGCCCTGTAGGCCTCTTTCATCTCTATTTTTTCATTTTTTTGAGACA
GTCTCACTTTGTCACCCAGGTTGGAATGCAGTGGCATGATCATAGCTCATTGTAGTCTTGAACTCCTGGG
CTCAAGTGATCCTCCCACCTCAGCCTCCCGAGTAGCTGGGACTACAGGTGTGAGTTGCCACCCCTGACTA
ATCTTACTTTTTTTTTCTGTAGATGAGGTCTATTTTACCTAGGCTGGTCTCAAAACTCCTGGCCGCAAGT
GATCTCAAAGCAAAGGAATCACACGCATGAGCTGCTAGGCCCAGCCACTTCCATCTTTAAAGTGGATTTT
TGGGAGAATGTTAGAAAGATTTAATTCTTGAAGGTATACACTGTTGAGCATTTGTCACGTTTCAGTAAAT
AATTGAGTTCTTGGAGCCCAGTGGTTTTCTAGAAACTTTGGTGAGAAATCTAGACTTAAACCTTTTAGGT
AGAGCTGGTTACATTATCCAAGGTGGATTTATTCTAGCAAATACTTATGTGACTACTCTGCTAGACACTG
AGGTTCTCTGGTTGTAGAAATAAGTCATGCTGTAATAGACAACAGTAGATAATTTCAAAGAGATGTGCAT
GGTGCTCTGGGGCAGAGGAGAGACATTTCATCATCCATGAAGGGGTCAGGAAAGATTCTTTGGATGAGGT
GCAGTGCCATTTTAAGCCTCCAGGCCAGGCATAGCTGATCAGGCCATGTGTATCTCCCCATTTTACTGTT
GCAGCAATGCTAGGTATAATAGGGACAAACCTGTGGAGATGCCCGGTATTCCAGCAGCTCAATCCAGTCT
GATTTTTGTTTTAGCTGCTTCCCCCTAGTGTTGGGGAAGGGGAGTTAGGATGTATAAATTCTCATGTCTC
TTGAGGTAAAGAGATAAGGCTGTTCTATTCTTTATAAATATACTGCTGCAGGATGTGTGATCCTATCAGC
CTTACTAAAGAAAATGCATTTCTCAAATCTTAGAGAATATTTTTGCATCATACCTTAGAGCTATAAAGGG
GATAAAATGAATTAAAAGGTAATGTGAAATAATAGAGCCTCTAGAATCCCACACTATTTTGGCTCTGCTA
CTTTGTAGCCCTGGGGAAGGTACTTATGTTTCCTCATACATACATACAGTGGGTCTCTTGGCTGCCTTAG
TGTTACTGTGAGGTTTAACTTTGTACAGTGGGGTGGAATGCTGCCTGCTGTAGTAGATATTCAGTAAGTC
TCCCTTTTGTAGCATCATTATTCTCCTCACCTTACCAGTAGTGCCTTACAGACTTACACATATTTAAGAC
CGTATATGTGGAATTGTGATGAATTTACAGCAGTATTGTAGTGTTTGTTTATAAAATTTAGCTGATGGGA
AGCTATATATTATATTTGGGTCAAACACTGAATTTCAGCCTGTCTCCCATGGAAATAACCGTTTGGAACG
TGTTTCTTGTTCTGCTATGACATTTGGAGAGTTGATACTTTCTGAAATATGTAAAGATGAAAAAGTAATA
AAATATGTAAATCATTTCTACCCTGCTAACTGGCATTTAGGCAAGCCTCTAAGCATCTCTGGGCTTCAAT
TCCTTCAATTGTTGGATGAAGAGTGTTTTTGGTTTGTTCGTTCTTTTTTTTTTTTGATAGAGTTTTTGCT
TTTGTTACCCAGGCTGGGGTGCAATGGCGCAATCTCCGCTCACTGCAACCTCCACCTCCCAAGTTCAAGC
GATTCTCCTGCCTCAGCCTCCCAAGTAGCTGGGATTAGAGGCATGTGCCACCACGCCTAGCTAATTTTTG
TATTTTTAGTAGAGACAGTGTTTGACCATGTTGGTCAGGGTGGTCTCGAACTCCTGACCTCCAGTGATCT
GCCCGCCTCTGCCTCCCAAGATGCTGGGATTACAGGCATGAGCCACTGCGCCCGGCTGATAAAGAGTCTT
GACTAGACAATTTCCACATGTTTAGTTTAGCTAGTATTTCAGAATCTTAACCCAAAATACTTATTTATTT
TTATTATTATTATACTTTAAGTTCTAGGGTACATGTACACAACGTGCAGGTTTGTTACATAGGTATATAT
GTGCCATGGTGGTTTGCTGCACCCATCAACTTGTCATTTACATTAGGTATTTCTCCTAACGCTATCCCTC
CCCCAGTCCCCCACCCCCCAACAGGCCCTGGTGTGTGATGTTCCCCTCCCTGTGTCCATTTGTTCTCATT
GTTCAACTCCCACTTGTGAGTGAGAACATGCAGTGTTTGGTTTTCTGTCCTTGTGATATTTTGCTGAGAA
TGATGGTTTCCAGCTTTATCCATGTCCCTGCAAAGGACATGAGCTCATCCATTTTTATGGCTGCATAGTA
TTCCTTGGGGTATATGTGCAACGTTTTCTTTATCCAGTCTATTATTAATGTACATTTGGAAAAACCCAAA
ATATTTATTTAAAAAAATTTCTTTTGGCCGGGCACAGTGGCTCACGCCTGTAATCTCAGTACTCTGGGAG
GCTGAGGCAGGTGGATTACTTGAGGTCAGGAGTTCGAGACCAGCCTGGCCAACATGGTGAAACCCCGTCT
CTACTAAATACAAAAAATTAGCTGGGCATGATGGCGCATGCCTGTAATCCCAGTTACTTGGTGGGCTGAG
ATAGAAGAATTGCTTGAACCCGGGAGGCAGAGGTTGCAGTGAGCCAAGATCAGGCCATTGCACTCCAGTC
TGGGCAACAGGAGTGAAACTCCATCTCAAAAAAAAAAAATAGAGACAGAGTCTCACTGTGTTGCTATATT
GCTCAAGCTGGTTTTGAACTCCTGGCCTCAAGTGATCCTCCCACCTTGGCCTTCCAAAGTGCTGGGATTA
GGGCATGAGCCACTGCACCTGGCCAAGAATCCAAAATACTAAATTGTCTCCTTGTGCCTTTTATTATTGA
TGTACAATCCCTCTAATTAAACAGATTATGCTTATTTTTTGGTAATAGTTTGTAAACTAGGTGTATTTTA
AATTTGAGATTATTTTATTTTTTAATTTTTTTCCTAAGGGCAAGGAGATGTTAGACACTAGACAGTAAAA
AAAAAACTTCCAGTTGAGATACTGTCACTATTCCATCAGACCAAATAGGAGTTTTTATTTAAAAGCTTTC
TGTGAGGCTGGGCGTGGTGGCTCACTCCTGTAATCCCAGCACTTTGGGAGGCAGAGGTAGGAGGATCACT
TGAGGCCAGGAGTTCAAGACCAGCCTGGGCAAGACCCTGTCTCTACAAAAAAATGTAAAAAAGTAAAATT
AGCCAATTGTGGTGGTCTGCACCTTTAGTTCTAGCTTACTTGGGAGGCTGAGGTGGGAGTATCTCTTGAG
CCCTGGAGTTGAAGATTGCAGTGAGCTAAGATTGGGCCTCTGTACTCCAGCCTGGGTGACAGAGCAAGAC
CCTGTCTCTAAAAATTAATTTTTAAAAATAAAAAATGATTTTTTATGTAGGTTATAAAAATAATATGCTT
GTTTTTAGAAATTTTTTTGAGAAAAATGTAGAAGCATAAAGAAATATGTAAGAATTATCTGTAGTCTCTT
CTATTTAGGGATAATCAGTAAATAGTGCTGATTATTATGGGGTTGTCACCTCCATTTTACATAAGAGGAA
ATTGAGGTATAAAGAAATGAAGTGCCATGTTCAAAGTTTCCTGGCTGGTAAGTGGCAGAGCCTGGATTTG
AACCAGGAAGCCTGACTTTAGAATCTGCTCCTAATTGCTTGCTGCATGAAGAAGATAAAGTCATACATTC
TAGCACCCAGAGCTACTCTATAAAACTTTTTAAAATTGTTGTAGATGTTTTTGTATTTATATTAGGTATA
TAGATGTTGTTTTAGTGAATTTTGGTTTATATGACATGAATACTACCTCCATGTCAGCATAGGGTTTACA
TTTTAAATGGCTGCATAGAATTCTCTTACATGTTAACGAAAAGGAAAACTGAGAGATGGCTTGGCTATAT
TTTTATTTTAAATTGCATTTTTTTCTTTCTCAGACCTCTTAGGGATGAATTGCCATATTCTCTATCCTTT
TTCCTCTCAGATAAAGTTGTAAGTAAAATCTTGCTGTAAATGTAGACGAGATAAATCTGTTCTTCTTTCC
ACTCTTCCTCATTGTCACTCTAGCCTCCAAATTCTGCAGTCACTTCTGGTTGGTAGGTAATTCATACAAT
GCTGCCATTATTTTTTATTTTTTATTTTTGACACGGAGTCTCGCTCTGTCACCCAGGCTGGAGTGCAGTG
GCACAGTCTTGGCCCACTACAACCTCTGCCTCCCAGGTTCACCTGATTCTCCTGCCTCAGCCTCCCAAGT
AGCTAACCGGCATGTGTCACCACACCTGGCTAATTTTTGTGTTTTTAGTGGAGATGGGGTTTTGCCATGT
TGGCCAGGCTGGTCTCCAACTCCTGACCTCAGGTCATCGGCCTGCCTCAGCTCCCAAAGTGCTGGGATTA
CAGTTGTGAGCCACTGCGCCTGGCCAGTGCCATTATTTTTGAATGGCATTTTGCCACTTACTTTTTTCCT
AAGTATCTTTAAAGGAGACTGTCAAGCTAGTCAAGAACTTTGTCTTATAGCCAGCATGTGTCTTGTTTTA
ATAGAAATGGAAGGAAAGCATCCTTAGATATCCTTTTGCAGAAGCCAAGAGAGGCTGTGATGTTCCTAAG
GTTATACAGTTAAGAGAGTCAGGGTGATGAGTAGAAGGCAAGGGACAGAGTTTTGGGAACCAGCCTTTTT
TTTATAGGAAGGTGGGGAAAAGGTAGAATGGTGAAGGAGTTGAGAAGCTTGAAGACCTAGGAAAAGCCAC
CATTGTCATGAAAAATAATTTTATGAAAATGTTGGTAGTTTCTGCCAGGTGTATCTTCGAGGTGAAGAGT
TGCAAAAAGGACGTTCGATTTAGCCATTATTGTGCTTTAATATCCCTGTTGTCAGTATCCCTTTTGTCAA
GAGACAAAAGATAAATACACTTGGTAATTTAGGACATGTAAAGTTACACAAAATAATAAAAAGTAATCCA
GCAAAAAGAGAAATAATCAAAGATGCCTTGGAGAAATAGATTATGACTCAAGTCAATTCAAGAATAAATT
AATTAGCATTGAGAAAATACTGCAAGCAGTCTTAAGATGGTATGTTACCACTGAGGGTATCTTTTCCATT
TATAACATCTGGAAAGCCAAAGACTGAGATCTTATATATTTTTTTTAAACAGGTGATAAATACATGTCTG
GTGTTAGGGGCTAATTCTATCCTCTGTAAACACTGGACACAGACTTGTTTTCATCTGCTGGAATGTGAAT
TCTAATTCTGAGAGTATTTTATGTGCGAAAGGATTGGCAAATAAAATTATACAACTTTCAAAACTCTTCA
TTTGCTCACTAAAAATATGCTTACCTTAAATGCCTGTTTGTACACTTTGGTGCAAGGAAAATTTTGTAAT
TGTGCTCTTAATCCTTCAGCTCACCTTCTTCTCTGCATTTCAGGTTGTATCATCCTCTCTTCCCTCCCTT
AGTCGCTTGAGGTGGTAACTGGGGTCTTGCTCTTATGTGGGGCTGCTATAAATGATCAGGCATATTGTCA
CAGAGGCACATCAGGTTTTTTTTATTTTTATTTTTTTGAGACAGTCACGCTCTGTCGCCCAGGAGGGAGT
GCAGTGGCACGATCTCAGCTCACTGCAGCCTCTGCCTTCCGGGTTCAAGGGATTCTCCTGCCTCAGCTTC
CCCAGTAGCTGGCATTGTAGGTGTGTGCCCCCATGCCTGGCTCATTTTTGTATTTTTAGTACAGATGGGT
TTCGCCATGTTGCCCAGCCTGGTCTTGAACACCTGACCTCAGGTGGTGCACCCATCTTGGCCTCCCAAAG
TCCTGGGATTACAGGTGTGAGCCACTGTACCTGGCCTCACTTTTTAAATAAGACGTTCAGTCTCGAAAGA
GATTGACTTCTTCAGGGTCGTACAGATAATCATGGATAAAACTAACGATTTGGACTGAAAATTTTCTTGG
TGATTCCTTAATGAATTTACATCTTTGCTTTTCCAGCAGTGAAAAGGAAGAAGACGATAATGAAAAGAGA
CAAGATCCAGGTGATAACTGGGAAGAAGGTGGAGGTGGTGGTGGAGGTATGGAAAAATCTTCAGGTCCCT
GGAATAAAACAGCTCCAGTACAAGCACCTCCTGCTCCAGTAATTGGTAATTTTACTATTTCAGTTTCAGA
AAGATGTCTAATTTATATATGATTTTATATATGGTCCACTTTCAACTAAGTGCATGATTGTGGTAGGGTG
AGGAGGAGAGGCTCCTTTCCATTCCTGGTAGAGTGGGATGTGTATATATCTTGTTAAAGACTTTAAAATC
CTAAATAAAAGGAATCTGGAAGTTAAAAGACATGCTTTTGTCCTGTATGTTGCTGTAAAATTTATATAGA
TATAGACTAGTCGATTCCAGTTTCATTTATTGGAATTGAGCATTTCTTCTAATATTCTCGTTAAATATTA
ACTACTTGCTAATGGAGGAAAAGGTAAGGAAAAGACTTTAATTTACTAGAATCCAAGGTCAGCTTCATTT
AGGATTTTTAGTGGTAACTACCCTAGGGTTGGGTTTAACTCCAAAAGTTTTCATGAGAGAGAAAATTCCT
AAAGCTTTTATCATTTAAAATATTTTTGACAGCTGAGTAAAGACAGAAAGACAAGGGGCCCTTGTCTTTC
ACTTGTGCTTTGTCTTTTTCATTCCCTATTTAACTTTCCTGTTCTTTGAAGCATGAATTCTCAGCAGGGC
CAATTTTATCATTCTCAAGGTGGTAAAAGTTGCTTCTTATGGAATGAAGGAATCTTATACCTTAACAGTC
ATTTGTGGCCCACGAAAAGACTAGAATGCATAAACAGATGTATAGTATATCGCAGTATTAAAATTTCATG
GAGTGGGGGGCAGTTAGGAAACAGATGTCTGAAAGGTCTCCTTAGGGCAATAATGAAAGTTTAAGAGCAC
TACTTTAAAGAAGTGTATACTCATTATTTTATTTAGAATGTTATAAAGAATAACATATCACTCCAAATCT
TGTTATGCAGAAATCACAGATAACTACTCTATGTTAAATTTTTATTTTTATATTTTTTAGAGACAGGGTT
TCACTCTGGCCCAGGCTGGAGAGCAGTCATGCAATCATGGCTCATTGCAGCCCCTCAACTGCTTGGGCTC
AAGTGATCCTCCCACCCCAGCCTTCCAAGTAGACTACCGGGGCATGCCACCTGTTTATTTTATTGTAGAG
ATGAGATCTTAACTATGTTGCCCAGGCTAGTCTTCAACTCCTGGGTTCAAGCAATCCTCCCACCTTAGCT
TCCCAAAGTGCTGGGATTGCAGGTGTGAGTCACCACACCCGGTCTTATGTTTTTAAATCATCTTTAGAAT
CTTTTTGTTTTGTTTGTTTTGTTTGAGACAGAGTCTGGCTCCTGACTTCAAGTGATCTGCCCGCCTCGGC
CTCCCAAAGTGCTGGGATTACAGGCGTGAGCCATTGTGCCTGGCTAGAATCTTTTTTTCTTTTTCTTTTT
TTTTTTTAAGACAAAGTCTCACTCTCTTGCCCAGGCTGGAGTGCAGTGGCACAATCTTGGGTCACTGCAA
CCTCCGCCTCCCGGGTTCAAGCTATTCTCCTGCCTCAGCCTCCCAAGTAGCTGGGATTACAGGCGTGCAC
CACCATGCCCCGCTACATCTTTGTATTTTTAGTAGAGACGGGGTTTCACCATTTTGGCCAGGCTGGTTTT
GAACTGCTGACCTCAGGTGATCTGCCTGCCTCGGGCTTCCCGAAGTGCTAGGATTACAGGCTTGAGCCAC
TGCACCCAGCCCTGGCTAGAATCTTAAAAAATAACCACACTGGCATTTCATTAAAAAATTCTTTACATAT
GGCATCTTAATCAGTCCACCAATCAGGTAAAGAAGTAATTCATATTTACTGGTAGGGAAACAGGACTAAA
GTCCTATTCTGAGAGGCAGTCCCTGATGAGACCAGTCATCCAAGCCCAGCAGATTCCTTTTCTACTTCCA
GTAATTTGGCTTGTGGAACTCATCATTTACGTAGATTACTAAAGTTGTACGTGGAGGAGGAGTTAGAATA
CTACCCTAGAAAAATACAATGTTTTCACTTTTCTTTTTTCTTTTTTGAGAGCCGGAGTCTCCTTCTGTTG
CCCAGGCTGGAGTGCAGTGGCTCAATATCGGCTCACTGCAACCTCTGCCTCCTAGGTTCAAGCCATTCTC
CTGCCTCAGCCCCCTGAGTAGCTGGGATTACAGGTGCCTGCCATCATGCCCAGCTAAGTTTTGTATTTTT
AGTAGAGACAGGGTTTCATCATGTTGGCCCAGCTGGTCTTGAACTCCTGACCCCAGGTGATCCACCCACC
TCGGCCTCCCAAAGTGCTGGGATTACAGGCGTGAGCCACCACGCCCAGCCTGTTTTCACTTTCAGACATT
TTCTGAAATGAATTCCCTTCTGTGGTCTGACCCAGAGACCCTGGTATTGTTTTTTCTATAATTGTTAAAT
GATTGGGAATGCAAGAATAGCTTATTAAAATGCTGAGCATTTAAATAGTTTATCTAAAAATTGTTATAAA
ATATTAGTTACAGAAACCCCAGAACCAGCGATGACTAGTGGTGTGTATAGGCCTCCTGGGGCCAGGTTAA
CCACAACAAGGAAAACACCACAAGGACCACCAGAAATCTACAGTGATACACAGTTCCCATCCCTGCAGTC
AACTGCCAAGCATGTAGAAAGCCGGAAGTAAGTACTATAATTAATTGCATTTTGTTTGGGACTTCTGTTC
CTAGGCCAGGGAGTGGGATATAGGGGATGTGCATACCCACCCAAGGGAGAGACTACTCCTTAACTAATAA
AGCCTGAGCTTCAGCTGACAGGCAGGTGAGAATCAGGGTGGGACTTCATATGTCCAGAAGGGAGGAAAGG
GGGAGGGCAAAGAGCATTGGAGACACTTTGTCTTCTGTCTGCAAAAGTTTATCTTAGCCCTGGTTTATGT
TGTTTTATAGCAATGACAAGTTTTAATGTAGTTAAAATTCTTTTATAATTAAGTGGCTTTTGCTCCTTAT
AATATGGAAACTAGAAATATAAAAGCGCTGTTTAATTGTATTCCCAGTTAATGTGACTATAGACAGAAGA
TGTTGGCATTTATAAATCTAATATTAAAATAATCTTATTTCAGCAGGTACTTAAAATGAATGCTACCTGG
AATTAAATGACTACATATGGCACATCTACATCTTACTTTAGGGACATGAATTTTTTAAAATAAATAAAAT
GGGAAGAAATGTAAATGACATCAGATGGAAATGTGTTTTTTGGGGTGGCTTTTAGAATAAATCCCAGCCT
TTTCACCACTTTGAGAGCACAGCTGGCTTGAATTTAAACCTCCACGTGGACTTGCTCCTCTGCTCTGTCT
TAAAAAATGTGTAAGGGACCTTGGGAGGGCAGTGATTCACCACACTCGAGTTTCTTTACCTGATCTAATC
AGATCCACTCCCACAAAATCGATGTGAGGAGAAATCATTGACTAATGAGTAGAATTTACTTGAATGAGAA
AATTGTTACCCTTGTGCATAAAGAGAGAGATGTATTATCTATTACTTGCTAAAAGTAAGAGTCTTAGGAG
GAATGTCATTACACAGCTTTTAACAGTTTTCTTCAAGTTTGTCCTGAAATAGGGTCACAGTTAATACTGT
TCAGTAGAAGATAGGACCCTAGAGCTTCAATACAGCGTTCTGTGTTTTCTGTTTGAATTGAAGCCTTAAA
ATGGTTTGATAAGGATTTTCTATATGCCTCCACTCCTACCCCTCCCCAGGATTCTTCTAAGGGGTGGCTT
TTTTTTAAATTCAAGGACGAATATTTTCAAAAACCCTAGTGAAGAAGAAATATTTACTGATTACATTTCT
TTTCCCTTAGGGATAAAGAAATGGAGAAGAGCTTTGAAGTAGTAAGACACAAAAATAGAGGTAGGGATGA
GGTTTCAAAAAACCAGGCCCTTAAACTTCAGCTAGACAACCAATATGCTGTGCTTGAAAATCAGAAAAGC
AGCCACTCACAATACAATTAAGGAATGGGCTTTGCTAACCCTTCTGAGGTAACTAGACTGCAGCTAACCA
CCACCAACAGCCATTCATCATCTGATCTCTGCTGGATCTACAGACACCGATGCAGACCACTCGATTTCAT
GACCGGCCCTATTGCACTATGGAAGTTAAAGTGTCACGACTGCTCTATGCATATTGGATTTAGGGGAATT
TTCATTGTTACATAAATGTGTGAACTAGTTTCAACAGTGTTCTTTCATATTTACTCTGCAAATACAAAAA
ACCAAAACCTGCAGCCAGTGGTCATTTCAAAATCTTTTTATGTTCAGATACTGAGCCTTCATAAGGGTTG
ACTACCTCAGATTTGCTGCACTCATTGTGGACTTCATGTGGATCACAACTTCTGGATAAGAAGATTACAA
CTATTAAGTGTCGATGTGAACCTTGCAACCAGCTCTACTGGATTCTTATCAGAAATCCTGCATAAAAAGT
CAGCCATCTGGGTTCTGATCTGCTGTAAAAGATGAAGATTTAAGTGACCTTAATTAACCTGTCCTGTGCC
CTACCCTTAAGGAATACTCTCTGTAGTAGGCTGTTGTTATATTAGACTTCCTGGAACACACTGCTGAAAA
GAACTGATGTGTTCAGATCATCTGTGTAGGGCTGTGATTTGTAATTTAAACTAATTGTATTCTGAGGTAA
CCACAAAATAAATTCAACCAAACTGGGGTCCACCAAGTGGGGGAAGGGGAAGGGAGAGAATAATCTTGGG
GGTTTTTTTTTTTTGGTAATTTTTTTATTTGGATAGTGCTTTTTTGTTTTGTTTTTGTTCTGCATTAAGG
CCTTTTTTGCTTTGACTTGAAATAAGTTCTTTGACAGAGCATATTGCTTGGTTAATTAAGTAACCTAAAG
TATGCATTAGGATTGTGAAATGTCTCGTGAGTATGCCAATCCTGAGGGTGGAACCAAATAGCCTTTGATG
AAAAGGGCAGTGGATTCTGGAGGCTCTACTTCAGGTGCTGCTATAATGCCTCATCTAATCAGGACTAAAT
TGTGTAGGAAACTGCAGTGGGAAGAATATGCTTTCTGCTCAGGCTAAGAGGGTCACTGATCTGTCCTTAG
AAATTCAGAGTAACATGAGCAAAACCTCAGCTAAAACCCATTTAAGTGGCATGGATTGTGCATGATCTTT
GATAAGAATTCCTCATGTACTTGTGCCTAGTTTTTCAAGGTATTGGCTGTTCTATAGATGCAGTGATTGT
CCCAGCTAGCTCTGTTACCAGCCTTTTGGTGTGTCTTTATGTTCATTTGGAGAGTCAGGGCGAAAGACAG
GTGATGTAGCACTTCTGTTTTTAATAATTATTGCTTAAAATACCTATTAATAGTTTTGGGTCATTTAAAG
GGACTTGAGGAAGCTACCCAGGATTACAGAAGAGTGTCCACCTAACAAGATGGTCTGGCAGTTTCCTAGT
TTTGTATCTGGTTCAATAGAAATATGTGAAAGTGGTAATGTCATCATTTGATGCAGAGTCCGGGTTTCTC
TATAATAAATCCCTTTGCCAAATGCATGAGTTGCAGACTTGCTACTGGCAAGAGTGAAGCAAGTGGGTGA
GTAAAACTATTTTGACGTGGGAGCGTTTTCAGATAGGAGTTTAGTCTTGACGAAAGTGTCCGTGCAGGAA
TTGGACTCCGAGGAGGGTTACAGTATCTCCTGACGGGACCTGCCACTCGCATCTGGGCAATGTTGACATT
TGAGGTGGCAGGCAGGATGCCTGCCTTCTAATATATTTGGGTGAGTAACTGAGCCAGCCAAGGGAAGGTT
GAATGATTAAATCAGAAATGGGATTCTTGGTAAACTGAAGACTTTTATTTGGGAATGAAAAACCTTAAAA
AAATCTCTTCATCGTTGAACTGTGCATTTTCCCTGCATTTTTTCCCAACAAAATTTTGTTGGGGGTTATG
TTACTGAAGAATGAACAGATGAGTAAGTGGAGGTGTTATGTAAAGGCATATTGTACTCGAAATCTGAAGA
CCTGCAGCAGATTTAAATTACAACTCTTGTTATAACTTTTTAAAAGATTGTGAAAATATCAAAATATAAA
TGAATCAAGTTTTAATATACTGTATGATGGGTGGATGAGGCTGTCCATTGTACCATTTGTTTGAATTCTC
AGGCATGGTTTGGCAGTGCAAGAATTCTGTAACATTAACAAATTCAATAAAAAGTAAATATATGGATTTT
GGACTGTAATGGTTATTTTAAAGCCTTATTCTGTCTTTGAAAATGGTGAGTTGAGGTCACACCTCTCTTG
CTGTGGGAAGGTGAAGATGCTCTAAACTCAGGGGTGTACTTTTTTTTTTTTTTGAGACAGTTGCTCTGTC
GCCCAGGCTGGAGTGCAGTGATGTGATCTTGGCTCACTGCAACCTCCGCCTCCCTGGTTCAAGCGATTCT
CCTGCCTCAGCCTCCTGAATAGCTGGGATTACAGGCGTGCACCACCACACCCAGCTAACTTTTGTATTTT
TAGTAGAGACGGGGTTTCACCATGTTGGCCAGGCTGGTCTTGAACTCCTGACCTCGTGTTGCACCTGCCT
CGACCTCCCAAAGTGCTGGGATTACAGGCGTGAGCCACCGGGCCTGGCCAGGAGTGTACTTTCTGTTTCA
GAAGTGTTTACAGAGTACTCAAGCATAAAAAAGACCGATGAGGATTTAGGCAAACCAAAGACAGTAAAAA
AAGAAATAGAAAATTTAAAAAATACAACACGCATGAAAGTGGCTGCAAAGCTGTGACAAATATTTTTGGA
AAACTTGAAATACACATTTGTCCTTGTTGCTGTATTAAATTTTGTTACCTTAAACTATGGCAAAGGAATC
TAAAATCTTTATATAGACTAAGGATTTAAGGCTTTGTTCGATTAATCTTACAATTCTCAAAAACAGTAAT
TTTAAATTTCAGGATTTAGTTTGGATTATAGTCCCTCGATTTCTAAATTAGCAAAAGTATATTTCAGTCA
TTTTGGTAGTGGAGTTTAAATATGTAATGTTAACAATTTACTATTTGATTATTCTTTGTGCTTGATTGAG
ATCTTTTTTTTTTTTGACAACCTTATTTAAGATAGAACACACACACAAACTCACACTCTATCAGTCTTCC
CTGCTTTAATTTCTTCTATAGTTTTAACTTTTTAGATAAAAGTCACTCTTTTCAAAAAGAAAAAAAGAGA
CAGGGTCTTGTGCTGGAGTGCAGTGGCGCAATCATAGTCCCCTGCAGCCTTTAACTCCTGGGATCAAGGA
ATCCTCCCGCTTCAGCCTGCTGAGTAGCTGGGATTAATGTGTGTGCTACCAAGCACTATTTTGTTTTTTA
AGAGATGGGGTCTTGCTATGTTGCCCAGGCTGTTCTCAAGCTTCCTGGTCTCAAGCAGTCCTCCTCTGCG
TCCCAAAGTGCTGGAATGTTTTACAGCCACTTTGGGAGCCACTACACCTGGCCAAAAGTCACCTTTTGAA
GTGTACAATTTAAGAATTTCTTATATTCTCAAAGTTATGCAACCATCACCACTCTTAAAATTCTTGAACA
TTTTCATCACCCCAAAAAGAAATTCTATTGTTATTAGTCATCATTTTCTTCTTTCCTCCCAAATTCCTCA
GTCCTAGGCAGCCACTATTGTATTGTCTCTAGTTTCTTTTTTCTGTTTTTTTTTTTTTTTTTTTTTTGAG
ACAGTCTTGCTGTCACCCAGGCTGGAGTGCAGTGGCACATTGTCAGCTCAATGCACCCTCCGCCTCCCGG
ATTCAAGTGATTCTCCTGCCTCAGCCTCCCCGAGTAGCTGGGATTACAGGCACCCACCACCACGACTGGC
TAATTTTTGAATTTTTAATAGAGATGGGGTTTCACCATATTGGCCAGGCTGGTCTCAAACTCCAGACCTC
AGGTGATCCACCCGCTTTGGCCTCCCAAAGTGCTGGGATTACAGCTGCATGAGCCACCGTGCCCGGCCTA
GATTTTCTCATTCTGGTCATTTCATATAAACAGGGTTATACAATTTGTTTTTCAGAGACTGGCTTCTACA
CTTGTTTTTGAGCATGTTTTTGAGGCTTAGCATGTATCAGTGCTTCGTTTTTATTGCTAAACAATATAGG
TAATTGTATGGGTATACCACGTTTAAATTGTACATTCATCTGTTGGACATTGGGGTTGTTTCATTATTAT
GAATGATGTTGCTATGAACTTTCATGTACAAGTTTTTGTATCAACATTTCCAATTCTCTTGGGTATATAC
TTAGAGATGAAAGGCTGTATCACTTTGAGGAACTGCCAAACTCTTCTAAAGTAGCCTCACCATATTATAT
ATTACTATTCCCACCAGCAGTGTCAGAGGATTCCAGTTTCTCCAATATCCTTGTGATCACTTGTTTCAGA
TTGGTTCTAGTCTTCCGAGTGGGTGTGAAGTATCTCATTATGGTTTTATTTTGCATTCCTATAATGACTA
GTGATGTTGAGCATCTTTAAATGTGCTTACTGGCCATCTGTATGTCTTTGGAAAAATGTGTATTCAAATC
CTCTGCCAATTTCTAAATTGGTTTACTTATAATTTTATTGTTGAGTTGCAAGTGTTTTAAATTTCAGGTG
CTACTTCTTTGTCAGATACATGAATTGTAAACATTTTCTCCTACCATTTTTCCCTTTCTTAGAAGTGTCT
TTTGGAGTACAAAATTTTCAATTTTGATGAAGTCCACCTTTTCTATTCTTTAGTTGCTTGT

An exemplary DNA sequence is human Rab6b provided below.

gi|224589815: c133625432-133532336 Homo sapiens chromosome 3,
GRCh37.p13 Primary Assembly
(SEQ ID NO: 312)
ATCACAAATTCAATTTAATCTTTTAGGCCACAAACTGACAATATTATGAGTATATTTATATGATTATATG
ATTAGCATCACATGGTATTTTAGGCAAATACAACACTTGAAATCACTGCAATGGCAACAGAGGTAGCTAT
GAAATTATTACAGTAGTACAGTATGTACTACAGTAATTATATGCAGTTATGATTTAACATTATACCTTTA
CATTTGTTTACATTTCTCTCATGAACCTAACGTATACTGTTGTAAGGTTCTTGTACTATAGGTAAAGTGG
TATGATAGTATATGAAGGTAGGCTATGCTAAATCAAAGATGCATACTATAAACCCTATTGTAACCCCCAA
AATAACACCACAAAGTTATAGCTAACAAGCCAACAGAGGAGATAAAACAGAATCATAAAAAAATGCTCAA
TCCAATAGAAGGTAGAAATTGAGGAAGAAAGGAACAAAGAACAGATTAGACAAATGGGAAAAAAATAGCA
ATTGATAAATTTAATTAAAGCCAATACTATCAATAATCACATTAAATGTAAATTTTCTAACCATTCTCAT
TGAAGGTGATATTGTCAGTTTAGACAAAAAAGCAAGACCCAAATATTAATATATACTGCCTGCAAGAAAC
ACGCTTTCAGAAAAATTATATAAATAGCTTAAAAAGGGAAAGATTGGAAAATATACTATACTAAAACTAT
AAAAAGTTAACTGTAGTGACTATATTTATATCAAAATGAAATTTTAAGGAAATAACATTACCAGAGATAA
GAAGGGTCATTTTATAAGGATATAGGGGTCAATTTTAGGTAGGAAGTGGGACTCAACTTCAGAGGGTTCA
ACATTGGACCAGATTGAGGACTAGCTAAAACATAACCCGGGTGAAAGCAGCTTTCAATCAGACCCACCCA
CCAGTGTGCCATGTCAATTTACTGTTGCCATGGCAACACCCGGGCGTTACCACCCCTTTCCATGGCACCC
AATGATTCAAAAGTTACTACCCCTTCCCTGCATAAACCACCCCTTAATCTGCATGCAATTAGGAGTGGGT
ATAACTATGACTGCAAAACTGCCCTGAGCTGCTACTCTGCCTTTGGGACAACCCTGTTCTGCAGGACCAG
TCATGGAGCTCTAACAACACTGGAGCTGTATCACTGCCTGTTCAATAAAGCTGTTTTCTTCTACCTCTGG
CTTGCCCTTGAATTCTTTCCTGGGCAAAGCCGAGAACCCTCACGGGCTAAGCTCCTCTTTCGGGCTCTCC
TGCCCTGCATCAAATTTATCAAGAGAGCATAAAAGTTCTACACCTTTATGCAGCTAATACCAAAGTTTCA
AAGCACATGAAGCCAAAATTGATAGAACAGCAAGGAGAAATAGACAAGTCCACAATGATAACTGGTGATT
TTCAAATCCCTCTCTCAGTAAGTAATAGAAGTAGAAAATCAGTGAGAACATATAAGACTTAAACAATACA
ATCAGCCAACTTGATCTAATTTATATTTGTATAACATTTCACCTAACAGCAACAGAATACACAGTCTATT
CAAATTTACACAGAATATTTACCAAGATGTACCATATTCAGGGACATAAAGCAAGCCTGAATAAATTTAA
GGGATATAAATCGCAAACTTTGGTGTCTGATCACAATGAAATAAAATCAGAAGTAATAACAAACTATTTC
TTAAACATCTCCAAATATTTGTAAAATAAATAACACTGTTCTAAAAAAAAAAACCACCATAGATAGAAGA
ATAAATCAAAATTAAAATTATAAAAGATTGTGAACTGAATTAAAATGAAAACACAACATATTAAAACTGG
TGGGATGCAGCTAATACTTGGAATGTAATTTAGAGCAATAAATCTGTATATTAGAAAAGAAGGAAATTTT
AAATCAATAAATCAGCCTCCACTATAAGAAAGTAGAAAAATAAGAACAAATTAGACCCAGAGTAAACAGA
ATAAAGGAAATAATAAAAACAAGAACAGAAATCAATAAAAGAGAAAATAATAGAGAAAATCAATAAAACC
AAAAGTTTATTCTTTGAGATAATCAGTAAAATCAATAAACTTCTAGACAGAATGTTGAGAGAAAAAAAGA
AAAAATACACAAATTACCAGTATCTGGAAGAAGAGAAGTGATATCACTACAGGTTCTATACATATTAAAA
GGATAATAATATTTGAACAGTAATATTTGAACAGCTATATATCAATAAATTATACATCTTAAATAGAGAA
ATCCCTTGATAGACACAAACTGCCAAGACTACCTTAAGAAAATACTAGATAACCTAAAGATCTAATATCT
ATTGAAGAAATTTAACATATTTAAAAAACCTTCCCACAAAGAAAAGAACGAAATGGCTTCCTTGGTTAAT
CCTACCAAATTTTTAAAAACAGTAATGCCAATTCTACACACTGTTCCAGAAAACTGATGAAACCATAACA
CTTTCCCAAAAGGATTTGTGAGCAGAATATATTAAAGAATTCTCAGCTGGGCACAGTGAGTGGCTCATGC
CTGTAATCCCAGCCCTTTGGGAGGCTGAGGTGGGCGAATGGCTGGAGCCCAGGAGTTTGAGACCAGCCTG
GGCAACATAGCGAGATGTCGTCTCTACAAAAAATATAAAAATTACCTGGGTGTGGTGGCATGCATCTCTA
GTCCCAGCTAGTTGGGAGGTTGAGGTTGGGGGATCACTTGAGCCTGGGAGGCAAAGGCTGCAGTGAGCCC
AGATTGCACCACTGTGCTCCAGCCTGGGCGACAAAGAGAGACCCTGTCTCAAAAAAAAAAAAAAAAAGAA
TTTTCAAAACCAAACAATAAGAAAACAAATAACTGAAACTGAATTGGCAAAGTTTTTAAACTGTAACTAC
AATCAAATAAGATATATAGATGGCTAATAAATACATGAAAAGATGCTTAATACCATTACTCATTGGGAAA
ATGTAATTTAAAATCACAATGAGATACCACTATACATCTATTAGAATGACTACAATTTTAAAAACTGATC
ATATGACATGATTGTATATGTAGAAAATCCAAAAGGAATCTATAGATAACCTACTACAATAAATAAGTGA
ATTTAACATGATACTGGATAAGATCAATAAGATCAATCCATGATAAGGCCTCTACATAAAATTATAAAAC
ATCACTGAGGCAAATTAAAGATACAAATAAGTGGAAGCAAATACTGTGTTCATGGGTTGAGACACTCAAT
GTCATAAAGATGTCAACTTTCCCCAGTCTGATCTATAGAGTCCATGAAATTTCAATCAAAACCCTTTCAG
AGGAGTTTGTAGTATGTGTGTGTGTGTGTGTCTGTGTGTGTGTGTGTCTTTGGAAATTGAGACTCTGCTT
CACAAACGTATTTGGAAAATATAAAAAAGAAACAAGACAATTTTTGAAGATTAATTTACACTACTTACAA
CATAGCTATAGTAATTTAGAGAGTGTGGTGTTGGCCCAATGAAAGTGAAACAGACTTAACGGAAAAGAAG
TCTGGAAACAGACCACTTATAGTCACTTGGTTTATGAAAAAGGTACAACTGCAGTACCTTTTGCAGAAAA
GGATGATAAATGTTGCTGCATCAAGTAGATATCCTTATGGTAAAAAGTGGATCAATTTCAGGTGTTCTGT
AACCTGAATATGAAAGGTAATACAATGTAACTTCTATTAAATGAAGTAAAATATTTGCATGATCTTGGAG
TGGGCAAAAGTTTTTTAAACAGGACACAAAAAGTCCTAACAATAAAGGAAACAATACATTTGACTACATT
TAAAAAAGAATCTGTTCATCAAAAGATATCATTAAGAAAGTAAAAAGGCAATCCAGATTTTAAAAAGATT
CTTGTCATACATATTATTAAAAATACCAGAAAACATAATGTACTCTGCATGGCAATAAGAAAAGACCAGA
AAACCCAATAGAAAAATGTGAAAATACTTGAACAAACACTTCCTTATGAGAATATCTAAATGGATAATAA
ACATAAGATTAGGTGCTCAACACTACTAGTTGTCAGAGATATGCAGATTAAGACTACTGTGAGATATCAC
TACAAATCTACCAAAATATCTAAAATTAAAAGACTGATAAAATAGCAAGTGTTGGCAAGGATATGAAACA
CTGGAACTCTTACATACAACTAATGCTTTATATGACCACTTTGGAAAGCTGGAAATACCTACTAAATCTG
AAAACATACATGCTTCAGCAACCCCACACTTAGGTGTACCCAGAAGAAACATGTACATATGCTCACCAAA
AACATGTACAAGATTTTCATAACAGCAAAATTTGTAATAACCAAACACTGGAAACAACCTAAATATTTTT
CTACAGTAGAATGGACAAATAAACCGTGATGTATTCATACAATAAAACACTATGCAGCAGTGAAAATGAA
TTTACTTTGGCTACATGCAATACTATGGCTGAATCTTACAAACCCAATACTGAACGAAAGAGATTCAAAA
GAGCACATATTGTTTGACTCAACTCATCTAAAATTCAAATATGGGCAAAACCAATCTATGCTGATGTATC
TGAAGAGTTATTTCCTTTGTGGAAAATGCAGTGTAATGACTGGGAGGGGTATGATGGAGACTTCTGGGGT
ATTGGTAACATTCTTTTTCTTGACCTGAGCGGTGGTTACCAGGATATGTTCGTTCATTCTATTGAAATTC
ATTAAGCTGCGCACTTGTGATTTGTTGACTTTTATGCCTGTATGTTATGTTTAAATATAAGTGTGTATTA
AAATGTGTGTGTGGAAAGATGAAGTAGGGATACTTTTCCCTATTCCTCCTATTAAATATAACTGAGTACT
CCGGATATTGTGAATAAAACAAACATAAGAAAACTCTGAAAGGTGCAGAAAAGAAGGCAGAACCAGTTAG
AGACCCTGGGACCCAAGGAATGAAACAGTGATGAGCTTTTTGGTTTTTCTTTTTACTTCATATATATCTG
GGTCTTGGAGCTGAAGATGGTGGCAACCCAGACACATCAACAAGCACAGGCCCAAATCAAGCCCCAATAA
AAGCTTGATCTCTCTAGTCAAAGGACCAGGAAGGGTTTGTTTTACTCCAGCCAAACACTACAGAAAAAAC
TGTGCTGTTCCCACCCCACACCTACCTAGCCAGCAAAGTCTAAGTGATGAATATAGACTTCTAGCATTGC
CAGGCTATATCAAGGTGCCCCAACTCTTTGGCTGGGATGGGGGTATCACAGAAGGTCAAGATGGGAGCCA
GGACTTTTGCCTCCAGTGGGCAGTACCAAGGCCTCCCACCTGCAATGTCAGCGTAGACCACATGGGGAGG
CTGGACTTCTACCACCACCCCACAGTAATGAGGACCAACAAATTTTTGACAAAGGTGCAAGAGCAATTCA
ATAGAGAAAATATAATCTTTTCAACAAATGCTGCTGTAGCAACTGGACATCCATGGGCAAAAAACCAAAA
CCAAACAACAAAAAAACCCCTCCAGCCTAAGTCTTACACCTCACACAAATGTAACTCAAAATGGACCATG
AACATAAATGGAAGACATAAAACTATTTTTAAAATTTTTTCAAAAAACATGGGAAAGTATCTTTGGGACT
TAGGGCTTAGTAAAGAGTTCTTAGACATGGCACCAAAAGGCACAATTCATAAAAGAAAATATCAGTAAAT
TAGACTTCATCAAACTCAAAAACTTTTGCTTTGAAAGAGACCTTGTTAAATTGCTCGGAGTGGTGGCTCA
TGCCTGTAATCCCAGCACTTAGGGAGGCTGAGGTGGTAGGGTTGCTTGAGGCCAGGAGTTCAAGACCAGC
TTGGGTAACATAGCAAGACCTTGTCTTTATAAAAACAAAATTTAAAAATTAGCTGGGTGTGCTGGCGCAC
CTCTGTGGTCATAGCTACTTAAGAGGCTGAGGCAGAAGGATCACTTGAGCCCAGGAGTTTGAGGTTGCAG
TGGGCCATGATTGTGCCACTGCACTCCAGCAGAGTGGGAAAAAATATTTGTAAATCACATATCTGACAAA
GAACTAGTATCTAGAATACATAAAGAACTCTCAAAACTTAACAGTAAAGAAAAGAATCTAATTTAAAAAT
GGACAAAAGACATGAACAGACATTTCATTAAGAGGATATACAGATGGCAATTAATCACATGAAAATATGT
TGAACATCATTAGTCATTGGAAATGTAGATTAAAACCACCCACAATGAGATATTACTACATTCCTCTCAT
AATGGCTTAAAATATTGACAACAGCAAATGCTGATGAGGGTGAGAAGAGGCTGGATCGCTCATCCATTGC
TGGTGGGGAGTGGAAAGTGGTAGAACCACTCTGGAAAAGTCTGGCAGTTCCTTTAAAAAACAAACATGCA
TATGACCCAGCAAGTGCACTCCTGGGCATTTATCCTGGAGAAATAAAAACTTCCTTTCACACAAAAATCT
CTACATGATAGTTCATAACAGCTTTATTTGTAATGGCCCCAAACTGGAAATAATTTCAGATGCCTTTCCA
CAGGTGAGTGGTTAAACAAACTGTACGTCCATACTATTGAATGCTACTCAGCAGTAAAAGGGAGCTAACT
ATTGCTACAAACAATAACCTGGGTGAATCTCCATAGAATTATGTGAAAAAAAAAATAGGACATGCAGTGG
AATAGTAACCAGCAGGGAAAATGAAAGAACTGCTGCTACATGAATCAACAGATGGATCTCAGGAACATAA
TGTTAAGAAAAATGCAAGTCATAGGACAGAATATACAGTTTGAGACATTATATAAAATTCAAAAGATGCA
AAACAAAAAAGTATTGTTGGAGGACACATCCAAATGTGATAAAAACTCAAAGAAAAGCCACAGTTCTGGA
TTTTCAAGCTGGGGAGAGAGGGAGGGGCAGAAACACCTGAGGGCCTCAAAAGTACTGTAATATTTGAGTG
CTCCAGCCAGGTGAAGTGAAAAAAACAATCCCAAAGGGTGATATACTGTGTAATGCCACTTATATAAGAT
TCTTGAAATAACACATTTATTGAAATGAAGAACAGATTAGTGGTTGCCAGAGGTTAGGAAGGGGTTGAGG
GTGGGAGGAAAGTGGGTGTGGCTATAAAAGGGGAACATGAGGGCTCTTTGTGTGTCAAGGTACAGAACAG
AAATGTACCTTGACTGTCTTAATTCCAGTATCCACATTATGATACTGTAGTATAAAGTTCTGCAAGATCT
TACTCTTGGTGAAAACTAGATAAAGGATATACAGATCTGTCTTTTTTTTTTTTCCTGTTTGTGAATCTAC
AATGATCTCAAAAATTTAATTTAAATATATATATATATATATATAACACAGCAAAGGGCTCAGGAAAACC
AAGATCTTGAAGAAAATGAAAAATATAGATGATGCTGTAGTGCTATATGATCTAACGAATATCTTTGAAA
AATAGTTTGGCACAAAATTAAACGTACACTTGTTTTTATACCACAATTTACCTACTGACACATATCTTAG
AAAAACTTCACAGAGGCATCAAGAGACATGTAAAGAATGTTGATGGCAGCATTGTTTGTTAATAGCAGAA
ACAAAACTGAGTGCATTTTCCAATGTTAATTTCCTGGTTTTACTAAGCAATGTGGTTATGTAAGATGTTA
TCACTGGGGTAGCTTGGTGCAGGATACATAGTAACTCCCTGCACTGTTTTTTATAACCTCTATGTGAGTA
TAAAATTATTTAAAATAATTTTAAAAAAGGCCAGGCGTGGTGGCTCACACCTGTAATCCCAGCACTTTGG
GAGGCTGATGCAGGCGGATCACGAGGTTAAGAGATTGAGACCATTCTGGTCAACATGGTGAAACCTCGGC
TCTACTAAAAATAAAAAAATTAGCTAGACCTGGTGGTGCGTGGCTGTAGTACCAGCTACTCGGGAGGCTG
AGGCAGGTGAATTGCTTGAACCTGGGAGGCGGAGGTTACAGTGAGCCGAGATGGCGCCGCTACACTCCAG
CCTGGTGACAGACCAAGACTCCATCTCAAAAATAATAACAACAATAATAAAATAAAATAAAATAAAAATT
TTAAATGACACATTTTACAGAAAATCATTTAAAAATTGGGGCATAACTAGAATACTAACCAGCAGTGAAA
ATGAAAGAACTGCTGCTACATGAATTAACAAATGGATCTCAGAAACATGATGTTAAGAAACATGCAAGTC
AGGACAGAAAACACAGTTTGATACCTTATATAAGATTCAAAAGATGCAAAACAAAACAAAGTATTGTGAA
GGACACATCCAATGTGATAAAACTCAAAGAAAAGCTAAGATTCTGGATTTCCAAGTTGGGGAGGGAGGAA
GGAGAAGAAAGCACCTGGGGGCCTGAAAAGTACTATAATATTTGAGCCCTCCAGCTGTGTAGAGGGTAAA
TGGGCACTCAGAGTGATAGGTTTTCTTTGTAACTTACATATACACAGCACATACTAAATAGGAAGATTTA
AAGAATTTTTTTAAATGGAATGCACAGATTCTATGATATGGAAGAAACTCTTTCTAGTGAGGTTGCCAGG
CTGAGGTTCAGTGGCCCCATTTTCCACCTGGAGAAAGTCTGTCTGCTTTAGGGCAGGTTAAGGACAAGAC
TCAAAATCAGGGGAGATAAGCAATGATAGTTGGAGGAGAGAGACTTGGAGCAGAGCAAACTTGGAGTCTC
TAAGGCTCTCATTTCCACAGCATGATGACTGCATTCTGAGAGCAATGCCAGTATCTGCTCCAGCGACGTG
AGCCCGGTAGTCGGAGCCGGGTTTTTGTCATTTGAACTGGAAGAATTATGACAGAGTCCCCCAAAGTAAG
CTGGACCATAGTCAGCCCAGCTGGACCTGGTAAGCATATGGGATGACTTGGAGGCTTAGAGGTGCCCAGA
GGGGCCCTGCCCCAGCCCTCCCTCCCCTACTGCCATACCACCTTACATGTGCGGGACCAGATGATCAGAG
GAGGTACAGGACCTGCCTAGGCTTACCCAAAAGATGGTGCCTTTTCTGGCACACCTCAGCTGTTTTGTCT
TGCCACCGATGATCTCATCAGGTCAGGATGAGAGGGTACAGGGAGACAGCACTAGATGTTAGGAGAAGGT
GCAGGTTGGAGGAGTGTTGCATGGCTGGGTACCTTGGCCAGTAGCTCACTGGCCCTTCTTTGGAGGGATC
ACTTATAGAGACGGGAAGGGCTGTGACTTGGGAGTCTCCTCTCCTTTCAGAGCTTTCTTCAACCCCAAGC
TGGATATGGTCTCCTAAGAGAGCAGGAGCCCCAGAGAAGTGGGTGGGTGTCTGGAGGGTTACCCTGAGCC
TGGTGCCAGCCACAACGGGGCAGATATTCTAGGTGATGACTTACAATCCCTGGGGTTCCAAACTAAAGGT
AATTATGAGAATAGGACAGTTATTTTGATTTGAGTTCTAGATGTTCATTCAAACATTCAACAACTATTTC
TTAAACTCTGAGCTATGTCCTTGGGATGCAGTGGTGGAGCTTCAACTACTGCTTTTATGGAACTTGTGGT
CAGGTTTCTAACAAGAAATAGGAAAGAGATTCGAGGTGAGCACACCAGGGAGACAGATAGGCTTGCCCAA
GCAAGCCCCATAAGTCAAAAAGGGGGTAACAAGTGTCCACGAGAAATCTGGAGGTGAAGGTTGTGTCTCA
CTCCTCTTTCTCTTTCTATAACACATGCGTGCACACACCATCGTGTTCACCGACATGCAGATTATTATCT
CAAATAAATGCTCACTGAACAGCCATAAATGAACAAGACCACGCTGTGGCAGAACAGTAAGGGACAGCTT
TGTAAAACCTCAATTAACCAGCATGCCTTAGGCTCGGGCTGTCTTGGGAGTGTTTCTAGCACTTGGCGTC
TGCTCTTTGCTCTCACTCACTCACAACTTGGTCTGTGTGCCTCGATGGACCAGGCAAGTAGAGGCCAAGG
AGAGGTCGCCCACATGTCCTCAGATAGCTCATTGTCCAGATACACCTAATTGCCAGGGTGGGGAGTGGCA
TTGTCAGCCTGGCGACCCCGGGATCAAGAAACGCAGGGCACTGTCCTGGAGATAGTGGCACTGGCATTCA
GAGCGTCACCGTTCCTTCTCTCCCATGGGGCAGGTTAGGATTTGGCATAAAATGATCCAAATGAGCAGCT
GGTTTGAGGTAGGCGCGCCTGATATTGGCAGCTCCTCTCTCGCTGGCCAGGTCTGCGGGGGCACCCACAG
ACTCCACAAGAGGCCGCCCAAATAAAGGGTCTAGGCCTCGAGAGGGAAGGGCGGGCTAGAAGCCACGGGG
GGCAAGAGTCGAGCCCCATCTGCGCCAAGTACGCCAGGTTTCCCTAGGTCTGACCGCCCTGACGGGCCTC
GCCACACCCTGGCTCTCCCCCACCTTCGTTCCTTTCCGCCCTGTCGGTCGGACGACTGCGCTGTGCATCG
GTGTCTCGCGAGGTGGAGGAAGGGACTGGCATCTAGGTTTAAAAGACCAGAAACAAATGTCCCCTGACAA
TTTCTGTCATTTTCTGGATTCAAAAGAGGGGTAGAGTGGGGAAGACAGACGCAGCGGGGCAGCGAGACAC
GGTCCCCCCCGGGGGGCTCTCAGGGGTACGGCGAGGAGGTTCTCTCCTGCCCGGGTGCGGGCTCGGGTGG
GCGGGCGCACGCCCCCCAGCCCCGCAGCCCCGCGGGCCCCGCATGACATCAGAGGCTGGGGAGCGCACAG
AGCGCAGGCGCCGGCGGAGGAGGCAGGCGGGAGGGGGCCGACTGGCGCGGCTCTGGAGAGCGCTCCTCCG
CCCCGCGCGCCGTCCCTGCGCCCCGCTCCGCCCGCGCCTCTCTCCCAGCGCCGCGCCTCGGGCGTAAAGA
GCGCGCCCCTCGCACCGCAGCCAGTGCCGACCGCAGCAGCCCAGCCCCAGCCTTCCTCCGCCTCCGCCTC
TCTCTCCTCCTCCTCCTCCGCCGCCGGACACGCAGCCGCAGGCCGGGACCGGGACGCAGCTGGGGAGTCA
GGGACGCGCGCAGCCAGCCCTTCCCCCTCCGGCTCCCGCACCGCCGGCCGCCTCCCCTCGCCCTCCTACT
CTCCCCTCCCTGCTCCTTCGCTTTTTCCTCCTCCTCCTCTCCCGGCCCCGGCTGCCAGCACCATGTCCGC
AGGGGGAGATTTTGGGAATCCACTGAGAAAATTCAAGTTGGTGTTCTTGGGGGAGCAGAGCGGTAAGTAC
CCGGCTTAATCTCTTGGCTTGTCGCCGAGTCGACCCCCGCCGCCCAATCGCCCACCCCATTGGGACCCCC
GGCCCAGGCTCGGAAAGGCCCCTCCTTGCCAGGGTAGAGGAGAGGGACAGGGATGGGGAGGTCGGGGCGG
GGTCGGGCTGGGGAGGGGCCAAGGGTGAGGACGGAGGAGGCGCTGGAGCCTGGGGTGGAGGTGAGGGTGG
GCTCTGGGTGGGGGGCCCGGGGTGTGGGTGCAGGTGCGGCGCTGCAGGGGGCCCGGAGGGCGGCGGCTGG
CTCCGCGGCGCTGGCCAGGGCGTGAAGGAACCGGGCTCGCTGGCTAGCTCACACTGCGGCACCGGCTGCG
GGCGACCGCGCAGGTGGCTGGGTGCTGGCGGGGGTGGGGAGAGCGACGGGCGCCCCCCTTGGAGAACGAA
GCGAAGCTGGGGTCTGGCCGGGCCTCACTCCGCTGCACTGGAGGGGTGTTCCCTGCGGACCCTGCTGGGA
ACCATGCGAAAGGGCACGTCCAGGGCGTGGGACAAACAGCCACCGGGGAGAAAAGAGCGACACCTGTCTC
TGCTCGAAAGAGCGAGTGCAGGCCATGGGGGAACGGGGAGCCTACCTCCCCTGGCTCACGTCCGACTGGG
CACAGAAGGTTCTAGACCCGCTGCCCTTCGCCGGCGCTTGTTAGTCTCCTTGCTGCCCAGGGATCTGGGC
AATTGCAGTGAGGGCTACCCCAACTCGAGGCGATTTGGGGAATGATACAGGGGCACCTCACACCGAGAGG
CTGTGAAGTCTGGCTCTGGGCAGAGAAAGGCCCAGGCGACACGCCCTGCCTAGCTTCCCAGCACTCCTCC
AGAGAGCATCGGTGGATTCTTCCCACCCCCACCTCCCAGCTCACCGCCCTTCTTGGGGGAGGGGAGGTCA
CCCTGGCAGTCGGCCAGAGCTGGGGGAGGGGGAGACTGCTTCTCCGCTGCCCGGCTCCCTCCCCAGGAAA
ACATCTCTGAAAACCAGGTTAGTCCCTAGAGGCGGGAAGCCCAGATTGAAGGGCAGGGACCCCTCTGCCA
CCTGTAGGAAGGAGTGGCTTTCAGAGCATCCCTCCAGAGGCCTCTCGCATGGGGCTGCAGGGAGACAAAC
TGAGCTCTGGTCAGCTCTGCTCTGGGAGAAGGCTGTCCTTGCGGGACAGGACCCCAGCTCTGCCTCTGCA
GACCAGTCCTGTTCACCTGGGTTGGTGCCTCTCTGACAAGGGGGCCCAAAAGAAAGGCAGCTGTGGGAGG
ACTGGGTGGGCCCAGGAACAGTGCTACTGTGCTGTGTGTACATGAGGGTTATGGAGGTCACGTGGCTGGC
TCTTGTCTGCTGATACTCATTGCCTAAGATGGATGGTTCAGAGAAGGACGTTAGAAGGTAGTGTCCAGGC
CCAGTATGGAGATGGGTTTGGGGGACAGGCTGGGCATTTCAGAAGAAGATCCACTCAGCCAGCCAGCGTT
TGCCCTGGATTCTGATTGCCTGTTTTTAATAGATCCCAGTTTCATAGAGCTGACCATACCCTGTGTCTGG
AAGGGATTTTGGTGGTATTCACATCCCAGTGCTAGTCTCACCCGGCCTGGAGCCCTGGGAGATAACCTGC
AGGGATCAGGCAGCCTGGGGGTTGAATTCTTAGCTGTGTTGTCTTTAGTAAAATTTGAGGCCTCTCTGTG
ACTCACTTTCTGTCTCTGAAAAATGGTTGCTTGAAGACAATTCTCTTACTTCTTCATGTTTCTTTAGTGC
CTCTCAAAGTGCCTGGCCCCTGGTAGGTTCTCAGCAAACATTTGTTCTAAGTCTGCACAAGCTTCAGAAA
GGACTAGGAGTTTGGTGAAGAAAGTGTCTCTGGTGAGTTTGAGGGAACATCTGGGCTCCTTCAGAATCCC
TGATGAACGGAGGCCCCTGCTGGGCTCCACAGACCCCCTTTGCTCCCAAAATCACAGAAATGAGAGCTGA
AGCCTAAGAAATGAACCCACCACCTCTCCCATATGTTCAAATGGGAAGTCTGAGCCCCAGGGAACAGGGA
GGCCTTGCCTGGAGTCCCAGAGTCCCATTGTGAGTTGCTAATGCTTCTGGGCTGGAGCCCCACCTCTCAT
TTCCAGTCCAATATTTTTCACTAGCGTGTCATTGTGAAGTAAGATGACCTTGAATTGGGGAAGCCAACAA
CTGGGGAGCCTCCCCAAGAGGGAGTAAGGGTTGCTTCTGTCCAGCCAGGCTTATAGTTGGGGCTTGTGAC
TGGTAGGAATGCTGAGTAGTTGGTAAGGAGCAGGGTTGTGAGCCCTTGGAACATGGTCATTAGAGAGTCA
CTGCTATCAGTGAGATGGGCTGCTGGCCACCCTGGTGCCTTCTGTCAGGGAGTCCCAAGGCCCTGAAAAG
CCTGTTTCCAATTTGTTGCATTGTTAGTGGGTTCTTTTAGGTTTTACACAGCTTTTGAGTTAGGTAGGGG
ACATGTATGTGCATCTCTAAGCCTTATGGGATGGGGAGTCACCTCCACAGCCGCTTCTTCTACCTTGCCT
GCACAGTTTTGCATCTGTTGGTCATTTGTGCCAGCATTCTCTGCAGCCTGTGCAGTGGAGAGAGCCACCT
GAGGGCTGGCCCTCACGCTGGCCCTGGCTCGCTCTTTTTATATGACCTCAGAGGTCCCTTATCTTCTGTG
CATGGGGGACTTGGGCCAGCTGAGCTCAGCGGAGTCTTTCAGAGCAGCATGGGCTTGCCGATATGAGCCG
AGTGCCCTGGAGATGGGCCAGGGGAAGGAGCTGGAGGGATGTGGCTTTATCTTATTTCCTGTGCCTAGCA
TAATGGTGGCATTTCTCTAGCCTGGTGGGCAAGGGCAATGGATTCCCAAGAAGAGTGGCGTGCATGGACC
AGGGTGTGGAGCACACCCTGGGCCAGATAATCGCAGGTGGACATCAGGTCTTTGAAGATACCAGGGAACT
GGTTTGAATGGGAGGCCCTTGAGAAGCCACTCTGGAGGAAATACCTCAAGCCAGTGAAAGAGCCCCCCTC
CTGGCAGCTGTCCCCCTGGCCTGTGGAATATAGGCTGGCTGACAGTGTCAGCTTGGCACAGGCTGGGATT
GTGGGGAGGGTGCTGTGGACCCAGGATGAGAGATTTCCTTGTGGCAGGGAGGCTGTGGAGTCCCCCTGGT
GCCAGATGGCTCTCTGGGCCTATTGGGTATGCCCCTGTGCTGATGGCAGTGGTAGGAGACATGGGAGGGT
GACATATGTCAGTCCCGACTGGGGCATTTGATGGCGTCCGAGAAGACAAGCAGGAGCAGGTGTTTGAGGA
GGGGAGAGTGGGCCTGTGGGACCCACGTCTTCCTCCTAAGCTCTAATGGCATTCTGTCGGCTTTTAGGCC
TCACTTGGCACATCTCAGTGTGTGTGGTGGGAAGGGTCAGGGATGCGTCTGCTGTGCACTGTCTTCTTCT
GACCTCCCCTTCCCCAAGACTCTTCCAGCAAAAGCAGGAAGACTGGATGGAGACGCAAAAGCTCTGTCTG
CTTTCCTAGCTGAGGACGGCAGGCCAGTTTCTCGCTCTGACCAGCCACCTGCCTGCCCTGCCCCTGGTAT
TGTGCTGCTGGAGGTGCTGTCCATGTTGAGCATACTGTGAATAGCATTCCTTGGAGCTGTGCAATGAAGC
GACTATGCAGGCAGCTGCCTTTTTGTTGCTCAGTTGTTTAGTCATTTGTTCATGTATCCTTCCGTCCCAC
TCCTTCTCTCTCCTGTCCTCCCTCCATTATCTGTTTATTTATTTATTCTTTGAGCGTATTCCTGCCCTCC
TGGAACTTATAGTCCACTGGGAGCACAGACATTTGTGGATATAATTACAGTAAAGGTCTATGAGAGCCAT
GATGAGAGAAGAACAGCTGCTACTGGGGCCTAGAGCAGGGGCGCCTGACCCGCCTCTGGGGGCTGGTGTC
AGAGGCAGCTTCCGTAGGCTAGCAAGGGATGCTTCTATAGGCAGTAGGCTACTGCTACTTCATCTTTGGG
CTGTGTGTAGAAAGTAGGTCTTGGAACAAGCCCACCCTACCCAGAGGGCCTTGCCTGTTCTTCTGCTCTG
CCCTGTAGAAAGTGTATGAGGTGGAGGTTCAGAGGACAAGGGTGACTCTCAGGTATCTGGCTTGGTGGTA
CTGAGTTGGGGCTGTCCCTGTACTTTCAGCCCATTCCTCTGTGGTTAAGGGTTAATGATCATTCAGTGGC
CCCCACTGCCTGGAAGGCTCACTCCTCTGCCCGCAGCCTGACCCATCCTGACCCCTGCCCTCCCACTAGC
CACACCATGGAACACCTGCCCTCCTGCAGGGAGCCCTTCATCAGCCTGGTAGATGTGCCAGCAGCCCCTC
CTAGCAAGTTTCCTCCTCCCACCAATTTCCAGACTTACCTGCTCTTAGGTGCTCACTTCCACTCCTGCTC
CCCTGGGCTACCAGCCCACCTGGCCACACACCATGGCATAGCTTTGGTTTTCTCTGAACATTTCTTAAAT
ACAATACAATCATATGCTTCCCCATGACCTGTCTTCTGTTGTCTCTGTTAGCTAAGGATTTTAAAATGTT
AACTTTTTCAGGTACAAAAACCGTAATTGAATTTTAAGCATCTTCTGAGGTCTATGTCTTAGTCATCATT
TCACCCCTCCCCACCTGCAGGTAGTTTGTGCTTATTAAGTATCTTGATTTGAAAATGATGAGATTATCCT
TGCTGTGTGAGATTTGCTGATGGCCCATCAGTAGATTAAAAGTGAAGGAAGTTGAGGTGGCTTGAAATAG
GATTCCTATTTCAAATTCCTGACACCTGCTGCTTTTTTAAGCCAGATTTTTTACCAGCAATATCTTAAGA
ATCTTGGCTTACTGCAACCTCCGCCTCCCGGGTTCAAGCAATTCTTGTGCCTCAGCCTCCCGAGTAGCTG
GGACTACAGGCCCACACCAATACGCCCGGCTAATTTTTGTATTTTTAGTAGAGATGGGGTTTCACTATGT
TGGCCAGGCTGGTCTCGAACTTCTGACCTCAAGTGATCTGCCCACCTCAGCCTCCCAGAGTGCTGGGATT
ATGGGTGTGAGCCATGGTGCCCAGCTGGATCATGTAACCTTGGTCATGCGCAACTCACAATTCATCTCAA
AAAACCTGAAGTTCTTGGGGGGCACTTCCTCATTCCTAGTCTCTGAAGAAATACATGAGAACAACCAGGC
CACCAGAGGTGGAAAGGGACACACCAGACCTCAGCTTGCCAGAGCTCACTTTGACATCTGGAAGAAATCA
CCCTGTTGCCCTGAGCCTTAGCTTCCTTCTCTTGATTTCTCTCCTCCCAATTTTTCTCCTTTCCTCAGTG
GCTGGCATAAGAAGGGGCACCTTTGTGGAGGTTGGGTACAGGCCCAGAACCTTTGCTTTTTTATTTGTTT
AAACAGATGTGTGTGAGTGAGAGAGTCAGACAGACACCAGGATGAGAATTAACCTGTACTTGGTATAAGA
GGAAGGTGGCACCTTTAATCTGATGATGCATAGCAAGAGAACTTGCCAGCAAAGAAAAAAGAAACTTAGA
ATATGTTCTGGCTGCGTAGATGTAATAATAATTACTGTCTTTTGCATGATCTGATGATCATTATCATTTG
CAGCCCCCAGATTATAATGAATCACATTAAATTATAACAAAATAAGGCTGGGCGTGGTGGCTTACGCCTG
TAATCCCAGCACTTTGGGAGACCAAGGTGGCAAGGTGGGTAGATCACTTGAGGTCAGGAGTTCGAGACCA
GCCTGGCCAACGTGGCAAAATCCTGTCTCTACTAAGAAGTACAAACATTAGCCAGGCATGGTGGCGCATG
CCTATAATCCCAGCTACTTGGGAACCTGAGGCAGGAGAATCGCTTGAACCCGGGAGGCGGAGGTTTCAGT
GAGCCGAGATGGCACCACTGCACTCCACCCTGGGCAACAGAGCAAAACTCCATCCCAAAATAAATATATA
TATATATATATATATATATATATATATATATATATATATATATATATATAACAAAATAACCTTATTGTCC
TTTTGTTCCTTATTCTATCCCTAAAATCAGTCTATGACTATCTTTATAGATGTTGCCCCGACATTGCAAA
CTTTGCAGACTGGCCGGGGCCAGGCCCAAGGGTGGTAACATTTGTATCCCATCCAGGCTGCAGATTTCCC
CTGTCCTCCCCACCCAAGTCGTCCATTGTTGCCGAATGCATCGGCCATTGAATGTGTAGGGCTGTCTGGT
TTCAGTGTGGTTAACTTTGGGAAATTATTGGACAGAACCACTGGGGAAGGTGACAGAAGGCTAGGTAGAA
CAGCAGAATTCATAAACATTCAGGGTGGACTACCGGGAAAGCTGATGGGCATTTTCTCTGCGTCTGCCAT
TCTTTTCCATTTCTCACCACAGGGAAGAGAAGCCGAGGGAGCTCTGGTGGAGGTTGCCAGGGAGAAGTAC
CAGACACGCCCTGAAATGGACAGCTTTTGTTAGGGTGGAAAGACAGGCTGCCAGGAAATGGGGCTCCAAG
CAGGGCCTGGGGACTGGAAGCCCCTGGGAACTTGGAATGCCAGTCCAGTCTTTGTTTCCTAGAACAAGGG
AAGGAAAAGATGAAGACTCACAACTCTTAAGGACTCCTAGGATGTTCTTTACCATATCTTGGGGCTTGAG
ATTTCAAAATTGTTTTCATTATTTTCATAACTTTTGCCCTAAACAAAGTAAGATCTTTTTAAAAAATATT
GACTGATATACATAATTTAGCATTTTAATAAAGTGATTTATTTTTTGTAGAAAAACTTATTTATTCTGAT
CTCTTGGTAACAAGGCCAGAGGGAGCCCAGGTGACCAGATCACATGCTCTGGGCCAGCCATCACCTGTCC
CAAGGGGCTGACATACCTAAGGGACAGGCCCAAGTCCTGAGGAGGCAGATGTTACCTCACCCCTGAGGGC
AGAATAGAGCTTTCAAAGCAAAGGTTACTTCCAGGAAATGCATAAAAAGAATGGCAGCCTGTGTCCTTCC
TCACATGAGTTCACTCACGTGGCATTTATTGAACATCCACTATGCCGGAGGCTCCACACAGGGCACTTTT
TATCTTCCCAGCCACCCTGTGACGGATGTGGGATCATCCCTGTTTTGTGGGTCAGGAGGCAGAGGCTCAG
AGAGAGCTGGCCCTGCCCAAGGACTCACTGTGAGTGTGGCAGAATTAGAATTCAGACCCAGGTCACTGTG
AGAAACACAGTGGACTATCAGACCTCTTGCCTCAAAGGCAGCTTCTGGCCTAGGACTGGAGCGGAGGAGA
CAGAAGAACACTGAGGTCTTCTGGACACGAGGTCTGCTGTGCATGACTCGTGGGGAGTGTTTGTGCCAAG
CTCTTTGCTAAAGAGGAACCTGCGGGGCCCTGAGCAGGGAGGAGGGAAGCAGGGTCTCCGTGTGGGAGCT
GCTGGAGCTGGTCTTGCACAGCAGGCACCCTGTTCTTCCATTTGTGGTGCTTTGAAGGGAGTTGATAGAG
CCTATGGGGGCAGCTAAATCCCTCCCAAGTCAGCCCTCAGCCCACTGCCATGGTTCTAGCTGAGAACTAA
TTACTAACCAGGGAAGAACCAATTAAACTAAATGGCTGGTTCCCACCTACTCTGGGGTAACCCTTTCCTG
GCCACATCCTCCCTTCTGGCTTGGAGATACCAGGGCCCTGAAGACCCTGATTCTATTCCCGGTCTGTCAC
TGGCTGGCTGTATGTTGGATAAGCCCCAGCCCATCTCTGGCTTTCTCCGTGTATCCCCTCAAGGCTACTG
GGCTGGGTGACCCCTTGGGCCAGTGGAGCTCTAACTCTGGTTCTGCAATTCAGTGCCAGCTCTGAGTATG
AGGCCAAGCCAATAACACTCCCAGCCTGGAGACAAGCATGCTGCTGGTGAGAGGGGCTGCAGGAGTGAGA
TGGGGGATCAGGGGCTCTGCGACTGTTGCCCCCAGAACTGTCCTGGGTCCCACATGCAGGATCTTCTCCA
GCCTACCACCTCCTCTCCTGGGAGGCATTCCTGGCCCCCTACACCTGAGTGCAACCCTCCCTGGGGGCAC
AATGTGGGGGAGGGGCATGCTGGCTCCCTTCTTCTGAAGATGTGCTTGGCTCTGGTTGCCAAGCAACAAG
GATGGGGCTATCCTGCACTTCTGCTTCCAGGAGGTGTGTCTTCCACACAGAGAGGAACCATGATTAGGAC
GACAGGGCAGGTGACACCTCCCAGGTGCTCCTAGCCCAGGGGCACCCCTTTCCACAGCCTGGTGGGAGGG
GTAAGCTGGGAGGGCAGGGAAAGGACAGAGCCAAGAGGGGTATATGGTGTCCCCAGCAGACCTAGGAGGG
CTTAGAGACCCGGCCCCAGCCCCAGCTCCCACCCCCACCCCATCACCATATTGGTCCTCCTGCATCCTGG
GCTTCCACCTCAGCCCACCCCTCTGCTGAATGGATGGTGTTGGTGTGCAATGGCACCACCTGTGCTCATT
TTCCTCACCTGTGCAGGAAGTTGATCATTACCTTGTTGGGGTTCCAGGAAGATCAAGTACATCTATACAT
GGCATGTGATTAAGGCTCACATGATAAAAGCTCAGGTGATAGGTAATTTTGTTAATGAAATAGTAACAGC
AATCTCCATCTCCTCCCTCATTGCAGAAGTAGGGTGCGAGATTTTGAGAATCATTGATCATTTCTGTATT
GTGAACTGTGGGGTCTACATCCTCGTGATCTGGCAGGAAGAGCTGCTCATCCCATTTTATAAAGAAAGCG
AAGGTCTGCCAGGTGAAGGCTTTTACACAGATGGAAGGACCCTTTGACTCCCTGGCCAGTGTTTTCCCCA
GCTCCCCATCCCTGCTCAGAGACAGGCTGGCAAAAAGTGGGACAGACTCACCATGCCTGCTCCTCTGGGG
CCACCAAACCTGCTCAGGGGAAGAGTACCTCATCTTCACAGCTCCGTTACTTCCTGTAAGGAGCTCAGCC
AGGGTGGAGTATAAGGGCAGAGGCTTTGCAGCATTGGGGTTATATGCCTTCAGATGTGTGAGGTCGGTAG
TACTTGGCCCACAATAAGCAGGCAACAAATCTCAGCCAGTATTAAGATTAGTATTACATACACAAGCAAG
GCTATGGGCGTGATGTGCTTGGAGGAGTCAGGGAAGGCTTTCTGGAGGAGGTTTTGCATAGTGAGCAGGA
ACTCACCAGCAAAGAAGGGGAGGCACTGAGGGCAGAGGGCCTGACCATGCCCAGGCAGTGAAAGGCTTGG
GGTTTCTGGGGAATCGGGTGTGGTTCAGGGTGGTGGGATTCTGGTGCGGGTGGAGAAATAGTGGGGGAGA
AGGGGTTGAGCCTGGGGGAGTCAGTAGGGTCAGGCCCTGGAGGACCTCCCTGCAGGCAGTGGGGATCCAC
TGGGGAGTTTTGACCCAGAGGGACATGCTCATGGATTAGAAGGTGAAGGCAGAATGTGTTCTGATTGGAG
GCAGGAGATCCAGGTGGCTGAACTAGGTCAGAACAGAAACGTTCCGAGGATGGAATTTATAGAGAGGGCT
TGTAAACTTAAGCTTCCTTCCAGTTCTGAAGCTCAGCTCCACTGCAGATGTATATTTTCTTGATTCTTTC
TCTTAGAATGAGGCCTCAGGAACAATGAAGGATTGTAGGGGAGGGACTATTGTCTCTCTCTGCAAGGCCA
CACTGTACGCAGATGGACCAAAGCTAGCCCCTTTAAGCTCCCCTGTCCATCCTATCTCCACAGTGCCCTG
GGTTCCCTGACTGCTCTGGCCTTCCATGGGTGTTCTGCGGGACTGCTGGCAGCCCTTCCGGACTCCTCCC
TCTCTGCTACCTGCTCAGGTTGGATGTGGCATATGACCAGAAACTGGCCATTGGGTGTGTGAGCTCCCCT
ATCCTCTGGTCATTGGATGTGTGAGTCCCCCATCCTCTGGTCATTGGGTGTGTAAGTCCTCTGTCCTCTG
GTCACTGGGTATGTGAGTTCCCGTCCTCTGATCATTAGTGTGTGAGCAGCCCATCCTCTGGCCATTGGAT
GTGTGAGTCCCCCGTCCTCTGGTCATTGGGTGTGTGAGTCCCCCGTCCTCTGGTCATTAGGTGTGTGAGT
CCCCTATACTCTGGTCATTGGGTATGTGAGCCCTCCCTCCTCTGGTCATTGGGTGTGTGAGTTCCCGTCC
TCTAATCATTAGTGTGCGAGCAGCCCATCCTCTGGCTGTTGGATGTGTGAGCCCCTTGTCCTCTGATCAT
TGGTGTGTGAGCAGCCCATCCTCTAGCCATTGGATGTGTGAGTTCCCCATCCTCTGGTCATTGGGTGTGT
GAGTCCCCCATCCTCTGGTCATTTGGTGTGTGAGTTCCCCATTCTCTGATCATTGGTGTGTGAGCCCCCC
ATCCTCTGGTTATTGGGATATATGAGCCCCGCCGTCCTCTGGTTATTTGGTGTGTGAGTCCCCCGTCCTC
TCATCATTGGTGTGTGAGCCCCCCATCCTCTGGTTATTGGGATATATAAGCCCCCCCGTCCTCTGATTAT
TTGGTGTGTGAATCCCCCGTCCTTTGATCATTGGTGTGTGAGCCCCCCATCCTCTGATCATTGGTGTGTG
AGCCCCTCATCCTCTGGTTACTGGGATATGTGGGGCCCCCTGTCCTTTGGGCACTGGGTATGTGAGTCCT
CCATTCTCTGTCATTAGGTGTGTTTGTGTCCTGTCCTCTGGGCACTGGGTGTGTGAGCACCCCCGTCCTC
TGGTCATTAGTTATATGAGGCCCCCTAGCCCTGTTATGCAGCTTCCTGTCCCTCCCTGTCTCACTGGACT
TCTCTTCCAGTGGACACTTGATCACTTCCTTTGCTTTCTTGGATATTTTCCCCTATGCCTGTCTGCTGGG
TTCCATCACCCCAGCAAGCTCCGGAGTCCCCAAGGACTTGAGAGAGAGCAGCATTGTAGAAGGGAGTGCC
CCTTTCCATGCCTTGGAGAACAGAGACCCATCTTAAGCTGGCTGCACAAAAAGCTAAGGCTGAAAGCCAA
GGGGCCAGAGTAGAGGCAGACACTGCCGTTTTGAACCCTCCCCTAACCCAGAGAAGGCAGCCTGTTCAGG
TCAATTGGTGCACCCCCCTGCTCACGGGTCAGCTCATGATCTGCTTTAAGTTTTGAAACCCCACCCTCTA
AAGGCCTGATGACTTTTGCTCCCTGCCCTTCCTGTTCTTCTGCATAGGCAGTGCCCACTGGACGTTACTG
AGCACCACCTTGGTATTCCTTTGCCAGTGTAGCTGGTGCATGGAGTAATCCTAATTTTGCAGCATCAATG
ATTACAGCATTTTTCAGTCACCACCCTTGGCCTCTTTGAGCAGTGCCAGGTTGTGAGTTCAGGGTGGGTA
AGGAGAGCTGCCCCCTCATCTTGGTGCTCAGCAGAGGTGGCTCTTCTGCCTGAAGCAGCTGGCCCAGAGA
AAAACTGGTTTGGAAGGCTGGGCCACAAACAACACAGCCCAGCTTTGACCTCTTCCAGCCTCCTCTGGTC
CCTCTGTTTCCCCCAACCCTGGGGCAGGCAGGAGGAGGACCATTTTGGCTCAGGATTGGAGAGCTGCAAC
TTTGGCCTGTGCACCAGGGACAAGTCCCTAGAGATAAGGAAAACCTTTCTCTCTGGGAGTGGCTGTGCTA
ATTCTGAGAAAGGAAGAGAAAATGTTCTTTTCTGACCCTGTCTGTGAGTGGGGGGAATAAACACTGCATA
GACTAAACAGTAAGTGAGGCACTTAGAACACATTTCTTGTTTCTTTCCCCTGTTCAGTAAAAGAGGAGGG
CTAAGAGGGCAAACAGGGGAAGCAAGGGGCACTGGGGAAGAGAGATCCATAGAAGTGCTGAGACCGAAGG
GAGAGGCTCACAGACACTGCTTCCTACCAGACACAGATGGGGGCTTGTTGGCCATGCACAGGGCCCTTGG
GAGCCAGGGTGGGTCAAGGTCCGGGCAGGGTGCTGTCCATCCCAGGGTCCTAACCAAATTTAATGAAAGG
CTGGAGGTTCCCATGGAGCCACTGATGAGTATGGGGGCACAGGACGATGGACCAGTTCTGTGTTTGTGTG
GAGGACAAGATGAGGAGCTCCAGCCCAGCAAAGCAGGGGAAATGAGAAGAGAGAAAGGCTGTTACCTTGC
CTTGGAGGTTGAGGGAGGGTGTTTTCCAGCACACTGCCTCTGTGCTGCCAGCACTGGCTTTGAGGAGCAG
ACTGAGCTTTGTCTCTTCCTCCCCAAGGCTGTCTGGCTCTGCCTCACTGTGACCCTGCAGTTCCCAGACT
GTCTCTGGGCTGCTGTGAGTAGAGTCAGGTCTTGGCACTAGTGATCTTTGTTTGGTTCTGCACTTCTGTG
AACAGAGCAAACCTCCAAAGGGTTTCTTTTGGGGGAGAAACCACTCCCAAATTATTTTAAGGATATTAGC
AAATCCTACTTGTGGCACCAAGATCTGGAAATTATCCCCTGATTCTGATTCATTAAATTCCCCTCCCTCC
CCTACATTCAACCCAAACACTGCAGGCTGCTGCCCAGATTAGAAGTGAACTGGAGACCTGAAGAGGTACC
TAGCTAGCCACCTGACCCTGACTCTCACCCATCTTCATCAGAAGAGGATAGCACCCCAGATGTTGGGCCA
GAAGAACTCAGCTCCTCTCCTGCCTCTATCATGTAGCAAGAAGACCACACTCAGCTGGAGTTGTGGCCTG
GGCAGCTGTGTCTGCTGGTCCTTCCACCTCTGCCTGGGGGAGGGCTGGGTGTGATGTGGGAGAACTGGAT
AGAGCAGCATGCAGATGCCCAGTGAAGCACACAGGCAGGACTCAGCCTCTGCCCAGGCCAGCTAGGAGCG
CTTTAGTCTCAAGGCAGAGACCTGTGAGGTTTTGTTCCAGGTGATCAGCCCGGTTCACCACAGTGGGAGA
AAAGGCAGAGGTGGAGTAAAGAGTAAAGGCTGCATCTCTTAGGAACCTGACTGTGAGATGGTGTGGTAGA
CACAACCTAAGGTGACCCCAGGTGAGTCATACCCTTGCATAGCCTCCTCCCCAGAGTGCAGATGGAACTT
GTGACTTGCTTGTAGCAAATATAGTGTGGCAAAGGTGATAGGTGGCATTCCTTCAATCAGGTTATGTTGT
ACGGCAAAGGTTCAAAGGTGATGGGATAGTCCCTCCCATGATTATGTTTCTTTATGAGACTCCTCTTGCT
GGCCTTGAAGAAGTAAGCTGCTGTGCTGAGAGAGGGCCTGTGAGGGGGTCACGTGGCAAGGGATGTGGGT
GGCCTTCAGGAAATAAGAGCAGCCCGTGGGGGGAAGTCCTACAACCACAAGGAACCAAATTCTGTCAACA
AGCACACAAGCTTGGAAAATGACTCCCTGATCCAGAAAGAAATGTAGCCCAGCTGACACCTTGATTACAT
CCTTGTGACATGCTGAGCAGAGGATCCAGTGAAGTCATGTCCAGACTCCTGACCCATGGAAATATGACAT
AATAAATGTATTAATATATTGTTTTAAGCCACTGAATTCCTGGTATTTGTAAAAGCAACAGATAACTAAT
ACAGATAGGGTGAAGATTTTAGAGGAAGGTGACACCTGAGATGGCCTCATTTTACACAGAAGACATTGGA
GTGTCCAGGAGGAGCTGAGACCTGTCAGGTTAAGTAGCCAGCCTGGTCCAACCTGTGAAGCACTCATTGG
GGTCAGGCCCTGGTTCTTGATCTGCTATTGTTCTTTCTTCCTGTTCTGTCTTTGTGTTCTGAGTAAACCC
TCCTTCTTTCTAGGCCTCAGATTTTTCCTCTGTGGCATGCAGGGCTAGACCACATATTCTCTAGGCCCTT
CTTGCCATGACGTGATTTTCTGAGGTCACTCTGATCATCTGTGATCCTTCTAGAAGCTGGGCCTCCAGGC
ATTAGTGTGGTTCACTCTCTGCTGCTCCTTGGCACTTCCCATGTTGTGTGTTTCCATTTGGGCCTCATGA
CACAGGGACACATTGGTAAGCCTTCTGGGCTCAGATTAGTGGCAAAAGCAGGAGTAAGAAGAGCAGTCTC
TGAGGTCAAGCCCTTGGCTGTGCACCGAAAGAGATAGTGTCTGGCCATGACCCTGGTGCTGTGTGATCCT
GGGCAAGTGGTCCCTCTTCTGTGGCCCCTCTTCACATCTCCTCTGATAAGATCAGCTCCAGTCTCCTCTG
TGGCTCATCTATAGTGCTGCTTTTGAGAAGGGCTAACCTTAGGGACCCAGAGCTCGCAGCATGGGGACAG
GTGAGCCTGGCCACCATCTCCTTGCCCTGGTGCCCAAGCATCCAAGTCCAGTGGGGGATATGTGGACATT
GACGCAGCTCCCCTTGATCCACCCCCCAGCCTGGCCCTTTGTTGACCCTAATGCCTGACCAGCAGTCTCA
GTCCATAAGGGCTAGAGACACAGTGGGCCTCCTGGCCTGCTTGGTGGTCCTGTGCCTACTCCCGGGTCCA
GGGTCCTTGGGATCTAAGTGCATTTTCCACCCCAGAGTCAGTGGTCTTCATCTCTTAAATTTCACTGGAA
AGGCCATCACTACTCTTGAATCAATCCTGAGCAGGTGACATTGAGTCCTTCTGGGGGCAGGAGGAGAGTT
CTTTCTGTTGACTGAATGAGAGGTCTAGAAGTTCACTCTGCCCCTTGTGCTCCCCTCACCACCTGGCTCT
GCAGGGGCAGGGGCAGGGCCTGTGCCCTCCTCCTTGGCAAAGTAGAAAAGTTGAGCAGCAGCGAGCAGGC
TGTGTGAGGGTGTATGGAAGGACAGGAAGAGAAGCTGGGGCTGCAGAAGTGCAGGGACAGCTTCCTGTGT
TGATGGAGACCGCAGGGCTTCCTGGGTTAGCAGGTCAGCACTATGGCTGCCAAGGCCAAGGGTTCCTCTG
AGAAGGGGGCAGGCATCCGGGATGATGCCTTGCCTGCATCAGCCTGCAGTCTAGAGGCCTCCTTCCACTT
GCCTGAGCAAGCTTTGTCTCATGGCGAGGCCACAGCTGTGTCATCACTGTTGGGTGTTACGGGCCCATTG
GCCTTGTCACAGACCTTCTGACTGGGTCTCAGCTGACAACCTGGGAAACACAAGTTTCGCTCTCAATACT
CTGTTTACTCATTCTCCCTGCCTCAGCCTTCTTTTCCTATTACTGAGTCTTTTAACATCAACACTACTAG
CAGTTAAGCCTTGGGTATATAAGCCTTGGGTATTAAAGCATATACATTACTTTGCCAAACTCCATGCTGG
TTGGATGGGCCAAGACTTCTGTCAGTTAAATAAGCTACCAAGTTACATTAATACTAGCCTTGTCAAAGAT
CAGATGGTGGTGGGAATTTAACTTCCTCCTTCTCTACAACTATGCATACTCACTGTGAATTAATTCTTCA
CAGCACCCATGCAACTGCTCAAATGCACAGGCAGGATTGGCCTGTGTAGGGTCTGGCCAGAGTTGGGTTG
AGCCTCAAACCAATTGAAATAGACTGTATTCTGCCTGCTTCAGTCTAGCTCTCCAGGGAAGCTATGGCAG
AGGGTAACTGTGTGTGCTAAGGATGTAGACTCTTGTTCTCTGAGCTATGAGAATAGGGTGGCTAGCATTC
AAAGCTAGCTGGTGCTGCTGCTGCTCTTAGGTCCAAGAGGAAAGATCCTGCTCCTTTTTGAAGCTGCCAA
GTGTATGTGAGAAGGCTTAGCAAGGCAGAGAGGATTCAGAACATAAGTGAGCTGACCATGGAGGTATGCA
GTCTACACAAGCTGTTTGGTCAGATCTCAGGTGATTTTCAAGTAACCCTGAAGGTATTGCCCTTGACTGT
CGGTCCTCATAGCTAAGACACTGGGGAACTAAAGAGTTCCTACCATACCAGGGCCTGTGATGCCTGCTGA
GTTGAGCATGTGTGTGTATGTGTACTTGCATGCATCTCTGTGCATGTGCAGACATGGTGGTTACACATGG
ACCCTGGCCACTGAATTGATTGATCCAGGCTTTCTGTCTCCTGGCCTGTGGTTAGAAGGGCGCAGAGCAG
TTCTCCAGTGCCCAGCTAAGTCTTGGTCCTTCATGAAGCCTGAGTTGATGCCAAGTACAGTTATACTCTA
GTTCCTTAGGAAAATTTCATGCTGACCAGAATGTTTAATTAGTCAGAACAAGCCATTTTCTGAATGTGCT
AGGAATCTAAGAAACATGGATGTGTGCGTGTAATTAAAATAATTTTTTCTGTAACTTTTTATTTTGACAC
AGTTTTAGGATTATACAAAGACTTTACATATGCCTTTCACCCAGATTCCCTGAATTTTAACTTGTTACTG
CTTTCATAGAATTCATAACAATTCTGTGAGATAGGCATTTTTGTCCTTATTGGATGTATGAAGAAAGCTG
AGCCTCAGAGAGAATAGGAACTAACCCAAGATCACACAAGAAATGAGTGGCAGAGCCAGGATTAAAACCC
AGGTCGGTCAGACTTTAGTGCACATGCTGACCCCATGTGATGTAAGTGTGAATGTGTGGGTATCCTGCTC
CTTCTGCCCACAGAGCTTTCTTTCTTGTATTGTTGATCTGCTAGCAGTGAATTGTCTCAACATATGCCTT
TATTTCACCTTCATTTTTGAAAGATTTTTGCTAGGTATAGAATTCTGTGTTGACAATATTTTTCTTATAG
TATTGCAAAGGTGGCACTTCATTTTCTTCCGGTTTGCATAGCGTCTGACAGGGTAGCAGATATTTCTCTA
TGTTTTCTTAGGTCTGTGATCTAATATATTGTTGCTAATTTTGTTTAAATAGTCAATTATCTTTTAAAGA
GACTCAAATAATAGAAAGATCTTACATACACATGTAGTGACCATTTCCTGTGTTCATCATTCCATTGTAT
AAATCCGCATTTCCATCTGGTTTCATTTCACTTCTTCCTGAAGGACTTTCTTTAACATTTCTTGATGTGA
GAGTGTGCTGGTGATGAATTCTGTCAGCTTTTGTATGTTTGAAAAAGTCTTTATCTCACCTTTGTTTTTG
AAAGATGTTGTCCCTGGACATAGAATTCTAGGTTAACAGGTTTTTTTCTTTCAGTACTTTATAGATATTG
CACCATTGCCTTCTTGCTTGCATTGTTTCTGAGGAGAAATATACTGTCCTTTTTATGTTTGTTTCTCTGT
ACATAATGTGTCTTTTTTTCTCTGACTACTTTTATGATTTTTTTTGTCACTGACTATGCAATTTGACTAA
GATGTGCCTTGCTGTAATTTCCTTTATGTTTCCTGTGCTTGGGATTTTTGAATCTGTGGATGTATAGTTT
TCTTTAATTTTGGAGTATTTTCAGTCATTTCTTCAAATATTTTTTCTGTCACTGCCTCTCCCTTCATCCT
TTGAAGACTCCAGTTACATGGATGTTAGACCACTTGACATTATCATAGAGCTTACTGGTGTTCTTATCAT
TTATTGTCATTCTTTTTTCACTGTGTTTCACTTTAGGTAATTTTTATTACTGTCTTCAGGTTCATTGATC
TTTTCTTCTGCAACATCTAATCTGTTTAATCATGTCAGCCAGTGTAGTTGTTACCTCTGGAAGTTTGATT
TGTGTCTTTGAAAATCTATTTTTTACCCCGTGTAACTTCTTAAACATATTATACTATATGTTATTATACT
AACATATAGTATATAGCATATATTCTATATATAATAACTAAGTTATATATAATATATATTATATAACAGT
TATAATATATAATAACTATTTCTTAATATCTTTATCTGCTAATTATAACATCTCTGTCAGTTTGGGGTTG
GTTTCAGTTGATTGTTTTTTCTGCTCTTTATGAGTCTATTTTCTTGCTTTTTTACATGCCTGATAATTTT
TTATTGGATACCAGACATTGTGATTTTTGCCTTGTTGTGTGTGGATAGTTTTTCTGTTCCTATAAATATT
CTTAAGATTTGCTAGCTGTGGCCAGAGTAGGGCTCAGTGTATGGCTAATTATTACCCACTGTGAGGCCCA
GCCCTTCTGAGGACTCTCCCCAGCATCCCGTAAATGATGAGACTTTCCACTCTGGCTGCTGGGCAAGAGC
ACAGTCTCCAGCCTGGTGTGGATGCCAAGCACTGTTTCCTCTAGTCCATTTGGGCAGTCTTTCCCCAGTC
TCAAGTAGTTTCCTCATATACATTTGCTGATCAGTGCTCAGCCGAAGGCTTGAGGGAAACCCTTTGCAGG
TCTCTGCCATTCCCTTTCTGCATCTCCCTCTCTCTGGTATTCCATCTTCTGAACTCTAGCTGCTGTGTTT
TCTCTGAGCCTCAGCCCTATCCTTTCAACCTAGGGTTTCCAGTGGGTTTCACCTGGGTCCCCACTCCCTG
TGCTGTGGCCTGGAATTTTCTGAGGGCAGTAATCTGTGGGGCTCCCCTCCTCTGTTTCATGTATCTCAGG
GGTCATCATTACCTAATATCCTGTGGCTTGAAAATTGTTGTTTCATATATCTTGTCTGTCTTTTGGTTGT
TTCAGGTTAGAGGCTAAATCTGGTCCCTGTTACTCCATTTTGACCAGAGCTAAAGTCTTGGGACCTTTTT
TTTTTGTGTGTGTATTCATTTGTTTATAGGGAACATCAGTCTGGGCAGGCAGTGTTGCGTGGTCTGGTTA
TCAGCCTTGATTTTCATCTGGGATGTTCATCCAGTTGATTGTATAAATATGTAGACTTGCCCTCCAAGTG
GCAACAAAATTTCTGCACTCCAGACTCTTTTTGAGAACTTTCTGCTGCCCAGCTCTTATAAAAAGCTCAA
TATTGGCAATTGATATAGTTTTATCTCCTGAACACTGTGAATCTTACCCTTGGTTGTGCTTCTTTTTTTC
CCCTTTGACTGCCAGGGAGCTTAGACAGTTTCTTAGTCTACCTTCAATAATTAGGGAAGATTTTTCAAGA
TTTTTTTTGTAAGCAAATTTGTGGTCCAGCTTTCTCTTTTTAGCTTACATAACTTTTGCCTTATATTTTA
TCTCTCTCAGCATTTGTAGTTGTGAATAAGAGAGAGAAACTCTCTCTATACAGCAGATTTGAGCAAGCAG
TGAATGTATTAGAGGAATGATTGGGAAGTTCATAGAATCCTTGGGAGGGTCAGAGATCTGGGCTTGGAGC
AGCAGCCCGAGTCATATAGAACTGGTCCGGGGGAGACACAGCTCCTATGCTGTGTTCTGGCAGGTGCCAA
TGTTGAAATGCATGTCATGGCTGCCCCTGAATTTAGAATACAGAGGGAATTCTCAGTCTCACCAGATTCA
TGTCAGTTGAATCTGGTAGAGTCTTACGTGAACATCTGTTGGGAGAAGCCTCAGTTATTTGCCTACAAAA
TAGCTGCAGAGGAGGCTCCTACACTGGGGAGAATGGTTTTGGACCTCCCCAAACACAGTAGGGGTATTCA
GAGGGATTGGGAGGCTTTTCATTGTTATTGATACATAGCTACTTTATTCTGATACTATTTTAAATAATTA
CGTTTTCAAATCTTATTTTCATGAGTCATAAGTACAAGTACTCCTTGTGCACAGTAGTCGCATGCCGGAT
AGTACTGCCTCTCCCTCCACACGAGTGCGTTCGGTTCTCCTCTGCCTGATTTTGAAGCTGGGGTTGACTC
TTGGCACCAGCGTTTCTTAGGGTAAGGTAATGGGATGCATTCTGTGGGCAATGTACCCCAAGTCATTAGT
CCTCACAGCAGACTTGGCTGTAGCAGGTAGGGAAATGGATTGTCCCTAGTGCCCTGAGTGTCTGCTTCAG
GCTGGGGGACAAGAAGAAATCTCAAGTCAGGTGTGGACTCTCAGTTTAGAAGCCCCAGGGCAGGGATGAT
TTGCAGTGTCCTGAGAGCTCATAAGGCTCTATGGGAGTATGGAAGGGCTGAGGCCAAGACTGATGTGCAG
GCTGGGCCTGGGGGCTTTGGAAAGTGAAGAAGAAAAGCTGGAAATATCAAGTATATATTATTATTAACTG
GAGTCACCATGCTGTATAGTGCGTCTCCAGAACTTCTCTTATAACTGCAAATTTGTACCCTCTAATCAGC
ATTTCCTCCATTTCCCCACCTCTGACCCCTGGTAACCACCCTTCTACCCTGTGCTTGTATGTGTTCTACT
TTTTTAGTTTCCACATATAAGTGAGATCATGCAATATTTGTCTGTGTCTTGTCGAGTAGATCTTAAGTAT
TCTTAACACATGCACACACACACACACGTGTAACTGTGAAGTAATGAATATGTTAATTAGCTTGATTGTG
GTAATCATTTCACAATGTATATATAGTCATGTGCTGCATAGCAATGTTTTGGTTAACGACAGACCGCATG
TATGAAGGTTGTCCCATAAGATTATAATGGAGCTCAAAAATTCCTGTTGTCTAGTATTGCCTATGCTATA
CTTTATGTTGTTATGTTTAGAGTGTACACCTTCTACTCACTTTTTTTAAAAAGGGAACTGTAAAACAGCC
TCAGGCAGGTCCTTCAGGAGGACCTCCAGAAAGCAGTATTGTTATCACAGGAAATGACAGCGCCGTGTGT
GTTACTGCCCTTGAAGACCTTCTAGTGGGAGACGGAGAGGTGGAAGACAGCGATATTGGTGATCCTGACC
CCATGTAGGCCTAGGCTAATGTGTATGCTTGTGTCTTAGTTTTTAACAAAAAAAGTTTAAAAGGTAAAAA
AAAAAAAATTAATAGACAACATATAGAATAAAGATATAAAGAAATAATTTTTGCATAGCTGTACAGTGTA
TGTATTTTAAGCTAAGTGTTATTACAAAAGAGTCAAAATGTTAAAACAAGTTAAAAAGTTTATAAAGTAA
AAAATGTTGTAGTGAGCTAAGGTTAATTTATTATTAAAGAAAAATATTTTTAAATAGATTTAGTGTAGCC
TAAATGTACAGTCTATTGTAGTGTAATGTCATAGGCTTTCACATTTACTCACCACTCACTGACTGACTCA
CCCAGAACGACTTCCAGTCCTATGGCTCTGTTCATGTTTTATGTTTTTTACCATACTTTTACTGTACCTT
TGCTGTTTAGATATGTTTGGATACACAAATACCACTGAGTTACAATTGCCTACAGTATTCACTGCAATAA
TGTGCTGTGCAGGTTTGTAGCCTAGAAGCAATAGGGTACCATTATAGCCTAGGTGTGTAGTAAGCTGTAC
CATCTAGGTTTGGGAAGCATACTTTATGATGTTTGCACTACGACAGAGTCACCTAATGATGCGTTGCTCA
GAACTTATCCCTCTCATTAAGCAAAGCATGACTATAGATAGATAGATGGATGACCCTTGAACAAGATGGG
TTTGAACTATGCATATCCACTTAAAGGTAGATATTTTTCAGTAAAATTCACACCAGTGTGCCTGCCTGTC
CTGCCTCCCTGTCCACCTCTTTCACCTCTTCTCTGCCATTTTTGAGACAGCAAGACCAACCCTTTCCCTT
CCTCCTCCTCTTCAGCCTATTCAACGTAAAGACAATGAAGATGAAGACCTTTATGATGATCCACTTTCAC
TTAATGAACAGTAAATCTATTTTCTCTTCCCTATGATTGTAATAACATTTTCTTTTCTCTAGCTTATCTT
AAGAATAGAGTATATAATACATATAACACAAAATACCTGTTAATCAACTGTTTATGTTATCAGGAAGGCT
TTGGTCAACAGTAGGCTATTATAATAGTTAAGTTTTGTGGGACTAAAAAGTCGTATGAGGATTTTTGACT
ATGCAGGGTATCAGTGCCCCAACTCTTGCCTTCTTCAAGGGTCAACTCTCTATATATTAAAACGTCACAT
TTTACACCTTGAATATATAAATTTTTTTGTTTATTGATTGTACCTCAGTAAACCTTGGGGTAGAGGCAGG
AAGAGAGGAAGCTAGAGAGGAAAAGAAGAAAACCAAAGATGATAGCACCTTTTGCAGGGCAGGAGAGGGG
GCCGAGCCCTCCTCTGTGGCGGCTGGGGCTTTGGTCAGGTACCTGAGGATGGGGCCTCTCCCAGCCCAGC
TGTGCTGGCAGCATGTTTCTCTGGAATTTGCCTCTTTGTGTTAAAGGTGGAGCCTCTGGGGGAAAAAAAG
CAGGAGGAGGTCCAGGGTGTTGGTATAGGAAGGAAAACTGAGACTAAATCTTCCAGTCTCCAGCCAGTCA
CCCTGTGGAGCCCCAGCAGACCGTCATGTCCCCAGCAACCACAGAGCTGCTGAAAGTGTTCAGGAGGAGC
TGGAGGACCAGCCATCCTGGGAACACTCCTGTCTAGAATCTTAGTTTTCGTGATGACAAGGGACCTTTAT
TTTGCCTTTCCTTTTTCTGCTCTTGTCCATGTACTGTTACGTGGATGGGATCCTTTAGGATGCACTCGCC
CTGTTCCTTCTTGCTGTAGTGGGCAGGCTTACTGGGCCCCCTGTTTGGATAGAGGAGATGGGGTGGGTGC
AGGATAGTGAGACGGGGCACTCAGTGGAGAAGTGAGAGGGTCAGGCATCGGTCCGCCTCCTAGCTGGTGC
AGGAGAAGGGCCTGGGCCAGACCAGCCTGTGCCACTGTCTGGCTAACCCCTCCCTCCCTCACCTTCTTTT
GACTCCTCCCTCCCCTCTACCCTCCCCTTTCCATCTTCTCTGGATCCCTGGGTGAGGCCCAGGCGGCTGA
GCGTCCTCTAGCAGAGGGAATTAACACACTTAATTAAAATGTCCAATGAAGAGCTTTGCATATTTCTTAA
TTACCATCTGAGTCTTTTGGGGGATGGTTTGAAATGGGGTTTTTCACGTGTTTCCTCATTTAGCCTACGG
TGAGCCAGCCAGCCCCTCAGGGCTCTAGTGGGAGCTGCCAAAGATGCATGCCCAGTTTGGGGCTGCCTTT
TGGAGTCCAGGGTGGCAGGGTAGGGTGTGGAGCAGATAGCCCAGCTACCAAGCTTACCCACCTTTGAGGA
AGAATAGGGCTGGACTTTCTGTTTGGTGAAAACAGAGCCAAAGGAAACAAGGTTCAGATGGATATAGCTG
GTGTCTCTAACTGTTTCACTTGTCACCACAGGGCCTGGGGGCCGTACCTCACAGTCAGGCCAGGAATGGT
AGACTATGAGCAGAGGTTACAGGGATGGTGCAGGCCTCACCCCAAGGGCCCTGTGCAGCAGGGATGCGGG
TAGCCTGGCCTCCTTCCCAGCTGCATGGCAAGGGCCGAGTCCCTCTGCAGGTCCTTCCTAGCATTTATAC
CAGCAATTCCCACTTCTGTTCTAGAAACTGAGCTGATGGGATGGCTGAGTCAGCGGGTCTCTGCTAGGAC
TGGGTTACTGGTCTGCACAATGAGGAAGCAGACTTGCCTTTTCAGCCCTGAGCACCACCTTCTGAAAGCT
GCAGACATTATCTGAAATGGGCCTGGCTGCTGGGGGCTGTGGGCCGGGGATGCAGAGATTCTTTTCCTGG
CTCAGGGACTGATTTCCTGAAAACACAGGCATCTGAGAGGTGGGCAGCCCACCAAGATGGCTTAGAAATG
GCAGTGAGTTATGCCACATCTGTCCTGCCTGCCCTCTCCTCCCGGAACTCCTGGGGCCCTAGGCCCTGAG
CCCAGCTCTGTGGACCCCAGGAGGATCAGGATCCCACTGTTTGGCCAGGAGCTCTGAAGAGCTGGTGTGT
GTGGTAAGTGTGGATGCACAGCCGAGAGCAGACTGTGGGCCAGGGTTCTGGCTTGGCTCTGTGAGGGGCT
AGCTAGTGGGTGGTACACCTTCTGCCAGGTCCTGGCAAACCCTCCGTGCAGTCTCTGGCTAATGTCTGTG
CTCTGCCCCCTGCTCTCCCCAGTTTCTGGCCCTTACAGCAGCCTCTCCACCCACACCCACACCCGCTGTG
GAGGCGACAGGGAGAAGAGCTGTCCTTCGTGGAGGGAGGCAGTGTCAGTGGAGGAGTCTGGGGACTTACT
GCCCACTCAGCTTTCAGACTGTGATGTGGGCACAAGACAAGACCAGAAGTCCTCATAGCCACTGGCCCCA
AAGCTGTGGGTGGATTTGGCGTCACGTGGCAGCTGTGGAGAAATGTGGGCAAATGATTTTAAGCAGAGGA
ATCTCAGCCTCACATGACTGTCGTTTCATCCAGTCCCTTCCCCGGGAATGTTGTTCCCTGTGACCCCCTC
ATCTCCATCAATCAAGGCTCAGCCCAAATGGTGCCTTTTTCAGGAGGTTACTGTGATTTCCCCAGCAGAG
CGGCCTTTCCTTGCATGGGCCTCTCAGGAGACCCTTCCCGTATTGCCTGGCATTTTAGTATTTGGATCTG
GGTCTCACTTCACCACTGGATATGGACTGCTTGAGGCCAGAGGTGCATCTGCTTTGTCACTACAAATTCT
GTGCCCTGCACAACAGGACTGGCACACAGTGAGCCCCAGTGGGTGTTTGCTGAGTGGCTGGAGAAGCCTC
ACTGGCCTCTGCAGCTACCTAGAAACCACCAGAATGTACAAGAATCTGTTTACCTGGGACCTGCCAGTGA
GATGGGGGCCTGGGGCCCATGGGGCACTGGTGGAGTGAATGGCCCCTGTTGGTGAGGTCCAGGTAGGCAG
TGGTAGCTGATTGTGAAGGGCCAGCATCCAGTGCACCCTGCATTGGTGGGATTTCCAGGGTACATGGGAC
CAAGCTCCTCTGGACATCCCCATGGATGTGCAAGACCCCTGGGCCAGTAGTTCTCACTAGGGTAAGAAAA
ATAACTGTTTCATGCTGATGCCAATAGTGAAAAATAAGCTTACATCTTTAGGACAAGGGATTGAATGATT
ATTGTTAATTGTTTTTTCTGAATGCAGTGCTGGCATTTGTGCTTTTGCTCTGACAATTATGAAGAAGGCT
TTTATGAGTGACTGAATCCAAGGTGATTGCTTGGTGTCTGTTCCTTGTCCAGTTGTCTTGGGCTCAGGGG
ATAGCTCTAGCATCTAATGGTCATGAACCCAGGTCACAGGGAGGAGTTCTGGGCTCCTCTCTGAATCTGA
CCAGTCACTGGTCCTGTACCCTGCTCTCCTCCCCATTCTCTCCAGCCCCACCACCTCTGCATTGTTCACT
GAGATCCTCCATCTCTGGCAAGATTTCTGCCCCTAGTCTGGCTTCTGTAGGCCATTCTGTAGCAGAGTGT
CCCCACCCAAATCTTACCTCGAATTGTAATCCCCATGATCCCCACGTGTCAAGGACAGGACCAGGTGGAG
GTGATTGGATCATGGGGGCGGTTTCCCCCATGCTCTTCTCGTGATAGTGAGGTCTCATGAGATCTGATGG
TTTTATAAGCATCTGACATTTCCCCTGCTTGCCCTCACTCTGTCCCGCTGCCTTGTGAAGAAGGTGCCTG
TTTCTCCTTTGCCTTCTGCCATGATTGTAAGTTTCCTGAGGCTTTCCCAGCAATGGCAGACTGTGAGTCA
GTTAAACCTGTTTCCTTTATAAATGACCCAGTCTCACGTATTTCTTCATAGCAGTGTGAGAATGGACTAA
CATGTTTTCTTTTTTCTAAAGCACAGGTCTATTGCTCTGATTACCTAATGCTGTGTAACAAACCACCCCA
AAACTTAGTGGCTCAAGACAATAAAAATATTTACATCGCTCACGAATTTGTAGTTTGAGTAGGACCTGGC
AGGGAAGGTGTGGCTTTGTTCCATGCAGCACTGAAGGCGGCTTGAAGTCTGCCGGCTGGAGTCACCTGAA
GGCTCGCTCACTCACAGGTCTGCTGGTTGCTGCATGCGTGTCTCCCTCCTGTAGGCCTTCTTGGGCTTCC
TCACAGCATGGTTGCTGGGTTGCAGGGCTGGCTTCCAGAGTGTGTCAGTGTGAGCACACTCCAGGCAGAA
GCTGTGCTATTTTTTCTAAGCTAGCCTTGGGATTCCCACAGGGCCATTTCTGCCATAGTCACAGGCCTGC
CCAGATTCACCCCCTGATGGAATGTCAGGGTTAGATTGTAAGAAGAGCACATGGGATGGGAAATATTTTA
TGAGCCCACTATGGAAATTATGAACTGCCACACCTGTTCACACCATCCTGCTGAAGATCCTTTCTGAAGC
TCCCACTGCCCTTGGGCGAGAATCCATGCTCCTCCATGTGGCTGACATGGCCTGTGCCATGTGGCCCCTG
CCCCTGCCTTTGTACTTCTCTCTGTCTGCCCCTCCCTCCCTCCACAACTGGCCCCAGAGATGGGTTTGGC
TTAAATCAACCTGACAGCCACATGGAGCTTCCATGAACAATGTTTGCTACTCACTCTTCCTCACACACTG
TCTGGCTTCACTTGCCCTGAGATGAACCTCAGTCTGAGGGGCCTCCAAGTGTGTCCCCCATAGCAACCTA
GCCTGTTGCCTTCTGTTTCTTCTCACGAGTGTTTTCTCTCCCTGTCTCTGTGCCTTTGCACACATTGTTC
CCTCTGTCTGGAATTCCCCTTCACCCCCACCCACAGCCTTTCCCAGTCCCGTGGCTGCCCTGCCTCCTCC
TTGCAGGTGTCCTGACTGCCCGGCCACCTGCACCCCTGCCTTTGGGATCTGCCGCTTTGCTACTGTCAGA
CCTTTACATAAACCAGCACAGCAGAGTGATGAGGCATCGACTCTGGCTCAAATCTGTGCTCCTATCCCAG
CTCCACCACTTCCCAGCTCTGTGACCTTGGAATAATAACTAACTCATTGGATCGTGATGAGGATTAAATG
AGAGAACCTAAATAAAGTGCCAAATTCTCATCTGCTGCTGAATAGAGGCCCAGTAAGAGTTTGAGTTTCC
TCTCCTTCTTTCTAGCATCTCCTGGTAAATGCTCAGTAATGAATTTTCTCACCCTCATTTGGTTGAAAAA
GGAGAAATGCATCTCCTTGCTTAATCATGTGGGACTAAACTACCAGAAGATTACAAGAACATTGCTGAGC
CATGAGCAGAGAGAAAATAAAAAAGCTCAATAATGATTTGCCTGCTTATTTTTAATAAAACCTGCCAGTG
GAGCTGAATCTTTGTCCTCAGAAAAAGCTCCTCTTTGCTGCAGTGGTGTCCGAGAGCTGCGGTAGAAGCA
CAGCGGTGTGACCTTGAGAACAGAGCTGGCAGGAGGACAGCTCCCGCAGAAACTCCGGCAGCTGTTGCGA
GGCAGGGGGACACAGGAGGAGCTCCTGGCTCTTCCACCTGCCAGGCTGCAGGCCTGCTGGGATGCCCCAT
GCTACTGCTCCTCTGAGGCTGAGTGGGAGTGCCTGGTCAGGTGGCTCGGGATGGGTTCTGCCCACCCCTT
ATGGTTGGGGGACTGAGAAAGTAGCCAGGTAGATGCAGTGTGCAAGTTCTCCATTTGCAGGTTCTAGCAC
CTTCCACCCCGGGCAGTTGTTGAGGGCAGGTGTGGGCTGAGGAAGGGGTCTTTGCCATTTAGAGGGGAGA
GGAGCTTAGCACAGCACAACCCCTTACCCACCTAGCTCCTTTATGTGATACCATGGGCAGAGGCTGGGCA
GAAATGGACAAGAGGAGCTCCTTTTAATAAGCTCTGCCTCAGGTCTGCAGACACTTCCCCAGAGACAAAG
CATGGAGGACAAGGGTCTCGGATACCACTGCAGGGGTGTAACACACATTACCCTGGCAGAAGTGACGAGG
GCATGGGTAGTCTTAGCGGGCTCTGAGGTGGGGAAGAAGGCTTCAGCTCCGAGAAATGAGAAAAATGAGC
CTAGAGGAATAGTGTAAAGTCATTGCCTTTTGCAGCAAAGTAAGAATGAGTCCCTCATCCATTTCAAGCC
TGAGTTGGGGTGCTGGGCCCCAGATGTTAAAACCCTCACTTTCTGAGCCCTCCTTGTACCCCACAAAGCA
CTTCCAGGGCACCTCAGTGACCAGTTTCTGACCTTTCAGATTGGATCAAGCCCCTACCAAGTGGGGAAAG
GCCCCCAATTCCAGACTTGGCCTGGGACGGGCTGTTCCCTGGAGCGCCACAGCAGTGGCAATAATGACCT
GGCAGAATTCTGAACCCAGCATGATCTATCAAGGAGTACCCGGATAAGAACTGAGCACCTGGTCATGCAG
CCACATCCCAGCCCCACTCAGCTCTGACCCTGGTGCAGGAGCTGTCACCTTAATGCCCGTTGTGTGAAAT
GTGGATGACATTAGTGCCACCCAGAGTTGAGGGCTGGGAGGTATGGTACCCAGTGTTGCTGCAAGCTTCA
GTGGGCAGGTGCCACGTCTCCTCTGTTTACTGTTCTCATGTCAATTCTGTGCCTGACTCAGCCCCTACTA
TGTGCCTGGAACATGCTCTGTAAATGCTGGGGTCATGTGTTGCCTATTATGGTGACTGCGGGAATGCATA
GAAAATATCAGCTCTGGCCAGGACCCCACACCTAAAAGCTCCTCCTGCACAATTCCCAGTAAAAAACTTT
CAGGGGATGTTTTCCTGCCCCCTAGACCTGTGAGCAAGTGCTAATCCACTGCCAGTGGCTGGTGTCTCAT
GGAGAGGCACCTTGGCATTAGAAGGTGTTGCAGGGATTTCCAAGTTTTCAGCTACTCCATGAAATTTCTG
TGCCCTTAGTGGCTTTTTGGGACCCTGCTTTCTCTCCTCTCTGTGCTTGGAGGCCCTGGGGAGAAGAGAC
TCAGTGTCTTCCCATCCATCTCCCCACCCTGATCCCTGATGCATGCACCTTTCCCTAAGGTGCAAGACGC
AGAGCGTCGCTGCCTAGGGAAGTGACCAGCGTGCCCTGGGATGCCTCTCTTCCCAAGGAGGCAGTAACTT
TCCACTCCCTTTCAAAAAGGAGGATGCTGTGTTTTCCCCAATGCATCTGCCTCTTGCTAGTTCAGGGTTG
CCCCACTTTCCCCTCTGTGGTCCTCAGCCAGGTGCCTGGGGCCTCATGGCCTCAGAGCTGATCCTCCAGG
TATGGGAGGCCCCCTACTTCTCACCTTCATGTGAAATTTAAGGGGAGATTTAAAGCAGTCAGAATGCTCT
TTATAGCCTCGTTTCTATCAGAAGTTTAGAAAAGGCTGCTTCCAGGTAAAATTAGAAGAGATCTTGGGCA
CATTTCTGCTCACCCCAGAAATGTTGTTTTAAAAATGGGGCTGTTCTGACACCAGCTGGTACCCTACAAT
TCACTTCTGGCACTGACCACCCAGAGTTGGCGTGGTGGTCAGCGTTCTCCAACAAGGGCAGAATCCTGAC
CTGGCAGAATCCTGAACCCAGCATGACCTATCAAGGAGGACCTGGATAGGAACTGAGCACCTGGTCAGGC
AGCCACATCCCAGCCCCACTCAGCTCTGACCCTGGGCCAGGAGCTGCCACCTAATTGCCCATTGTGTGAA
ATGGGGATGACTTGAGTGCCGCCCAGAGTTGAGGGCTGGGGGTGGTGGTGATACATATATTTTTTCCATC
TGATATTCTGGAAACTGCCTTACCTTGCTCAGTTACCTGCTGCCTCCTTCCACTTTTAAGCATTTCCCAT
GTGCCTTGTACTTTTTACCTGATTTAATTCTTACTACAGCCCCCTGAAGTCTTTGATGTTAATATACTCA
TTTTCAAATGAGGAACTGAGGTCCTGGGATGGTGATAACCTCAAGGTGACACAGCTGAAAAGTACCCCAA
TAGGATTTCAATCCAGCTAACCAAATCTGGGTTAAAGATGGGGTCTTCTGAATTTAGCCCAAACACAGTC
AGTCAGATGCTCCTTCTGGGTAGAAGATCATCTTTCCCATAGCAAATGCTGGCTGCTGGGAGCTGAGTGC
ACTTGTCTGTTATGTGGTGCACTCTTGAGGCCTCAAAGCTGGTAAAGGGCCTTTGCCAAGGGGTCCTGTT
GGCTTGTCCTGCTCCCTCCAGGGAGTGACCAGCCACCCCACCAGGCACAGCTTGTGGATGTACTGCCAGC
CTGGGAAATGGGAGGGTGAGGGTACTTTTGTTTTGCCTGCCAGCATTACCTGTCCTCCTGAAGCCTAGTG
CAGAAAGGGCTCTGAGCCAGCACAGCTGGGCAGATTCCCCACTGAGCCTCCCAGGGTCATGGCAGAGACA
TGATAGGACAGTACAGACAGGCATCCGTGCAGGGCCCGTCCTAATGAATGTCCCTTGACTGCCCTCTTCC
CCCACCCCAGATGGTTAGAAATGCAGCAGTTGGCATTTCTGTCACTGCCAGGCCCCAGGGGAGCTGCACA
GGGTGAGGTCAGAAGCCAAATTCTACAATTTGGGAAATTAGCAGCATTTGTAGGATTCCCAAGAGACGTC
CTGTTTTCTCTCAGCTGTTCCAGTTGTTCTCAGTTGGGGTGTCACTGCCCCTACAGGGCATTTGCAGATG
TGTGAGTGAGTCTACAGTTGTCACAGTGACTGGGGATCCTCCTGGCATTTACCTAGCGGGGTTCCAGGTA
TGCTAAACATCCTGCAATGAGCTGGACGGTCAGATACACAAAGAATAGTCCTGTCCATGAGAGACATGGG
GAAATGACATTTGCCTCCCCGCCCCTGGTTCCTTAGAAAGCCACTGTCCCCACCAATCTTCAGGCTGAAT
TAACAGGAAATAAAGGGCAGGCTTCCATTCCAGTGTATGACCAACAACAAGGTGTACATGGGCAAGTACG
AGAGGAGCCTCCTGTTTTGCACCCACTTACCTTGATTCTTTATAGATGGTTCTTGCATCAGCTGGGTTCT
CCTGGGTCAAGGAAGACCTGGATTTGGGTAGTGGAAATCCATGGGGCAGTGGGATACCTCCTCCTTGGCA
GCACCCTGGTGCCTGGCAGGAACAGCCCTCAGCCCACCTGGTATAGAGACTCGGTGGCCTCACCACGGGG
TCCCTAGCAGGGGCCTGTCTCCCACTTCTGTATTTCTGCAACTCGGCCTTTCCCCTGTGCTTTTTGGTTT
TGTTATCTTTTCTTCAAATGCAGTGTGTTTAGAAGAGCCTAGCTCAGATGCCATGACTGAACACCTCTCT
CCTTGTTGTTTTGCAGTCGGGAAGACGTCTCTGATTACGAGGTTCATGTACGACAGCTTCGACAACACAT
ACCAGGTGAGGTCCTGGGTGCTCACCCTCCATATCATCTGGCAGTGGCTGACCTCTCTTTGAAACTTGCT
GAGCTGGGGTGGGAGTGGAATGGCCCTGGGTTCCCAGGCTGTCATTTTGCTATGGAGGAGCTTCCCACCT
CTATACTGGCCCCATCCCTGTAAAGAGCTTGAGCCCTTCTGAAAATCCACCCCTCGGTAAAGGGAACATT
TTTCAGAGGAAAATTCTAAAAATAGAGAGAGTCTGTGTTCTCTTGTTCCTCACAGGCAGATCCAAACCAT
GATAGTGTTTGGTTGTTCAGGGGAGTCATCATGTGGAAGAGGAACAAGATGAAAATGAGAGTGTTAGGCG
AACTTGCGCCTCATGCATGGTTGTAATGGTTATGTTTGCTGGGACCTTCTCTCTCTCTCACCCCCGCCCC
CCCCCACACACGCACACAAACACACACACGCGCGCACACACACACACACGGTTGCTGAGGCACTTCCTTC
CCTGGAGAGGCTGGTGCGATGAGCTTGCCCTGCCCTACATGGGCAGGAGGCTGGATGCGGTGACCTCTGC
AGCTCCTGGCCCTGGAGGCGTAGTCTGGGGTCAACCAGAGGCCCAGAGGTGGTTCCAGGTGCCCTGAAGG
CCAAGTTCTTGGGGGTTAATAACATGTCACTTTCCTGGCACCACTGGTGAGTAGGGAGGTTTGTGCAAGT
AAAGTGAATTTTCCATGCAATTATAGCTTTTTGCAAAAGGCACCAAACTTAATCATTTCAACTCTTTTAA
AGAAAAAAGGTGCTGTTTTATTCCTTAGATAGAGACAAATGATAATGCATTTTTCTACACTAGTGGATTC
TGTGAGATACTAATTCCAAGAGGTGTTGGTAGGTGTTACTTGAAAAAAAGTTGAGGTAGGGGGTGGTGGG
TTCAGAGCTTGTACAGATTTAGGGAACACTGGGTGAACAAAAGTAAATCTACTGCTGTGCTGCAGGGCTT
CTCGGAGCCTGACTATATCAGTGTATAGTTTTGATTTACTGAAAGAGGGATATAGTGTGTAGGATCTCTC
AGATGTATTTGATCATGGAATCCCTTGGTTATCGAGTTCTCAAAGGACAAATATTTATAGAACACAGCGG
GGACTGCTGTTCTAAGACGTCCCTCCTCCTTTCTGGATTTACAGAGTATGTGGAGCACAGCTCACTGCAG
ACTGCAGCTCTGTATCTACTTTTTAGTCTGTCTGTCTCCCTCTGGACCAATTATCCTCCTTGCCTTAAAT
ACAGCCCCTCTGTCTGCCTCTCTCTCATTTTTACTCTTTCTTTGCTGTGGTCTCAATTTTTACTTTACCT
CTGCTTCTACTCTGCTATGGTCTGAAATACTGGCTAGCCAGTGGACTTGGTGAAACTGACCATAGGAATT
GACAGAGGCTCTGGGAGAGGGGCCCTGGCTGATGTGCACAGCCTCAGGTGTGGACTGTGGGATGTTCACC
TTGGTCACACTTTGTCTTTGTTGTATCCTCTCTGGGCCAGGGCTTTGTAGCACCAAGCCTGTCCTCCTAA
TGAGGGGAGTTCTACTGGATCGGTTTGAATTCAAGATGACAGCTGCAACAGACTTAGTTCTGAAGTCTGA
CTTGGTATTCATTTATTTTTGTGCATGCATGTGTGTGTATGAATGATCTCATATATTATTTTGATTTCTT
ACTATTAAGTTACATATAATGCTTCTTGTTAGTGTCCCATTGAAAACAGTGAGACTCTTGGTTGGTTGGT
GTTCTTGGCAGTGGCAAGGGGGTGACAAAGGGGGCCCCCAGGCCTGTGCAGTGGGTCCCATTAGTATGGA
TCAAAGCCTCATCTCCTCCCCATGTCATTCAAGTTCCTCAAAATCTCGCCCTACCTCCATGTTCAGCGTC
GTCTCCCATTGCCTACTGCTTGTCCCCCATCCTGTGGTCCAGCCGACTTCCCTGCTCCTACTGCCCTTGG
AGTGACACTGTTATGCCTCCACACTTTGACTCATGTCATTGCCTCTGCCGGAATGCCCTTTCCTCTCTTC
TCTGCTGCGAGAATTCTCTGGATGCATCAAGGCCCAGAGCACATGTTTGGTCTGAGCAGCGAGGCCCTTT
CATCCTCTGTCCCCTCCGTGACCATCTTTCCTATAGCATCATCCTCAGGGCTGGTTTAGCCTCTCAACTG
TGCAGTAGCTCCTGTGTGCATCTGGCTCTGAGCTGGGCTCTGTGCTTGGCTGAGGCCAGGACCCTCTGGG
ACCCACCACTGCATTCATGAGTCTAGCACAGGGCCTGACCATAGTTGGTATCACCAGTGCCTGCTGGCAC
ACACACAAAAAAAACAGCATGGAGAGTTGGCCTCGGGTTTTAGACCCATCAGATCAAGAGGCCTCAAGGA
CCCAGTGTGGTGACAAGAGGAGAGAAACCTCAGAGAGGACTGCCCTCCCCATCGCAGGCTTTCTCTTTTT
TTTTTTTTCAATATTGCAGCAGGTGGAATTCCTTTCCCAGTGCTCTTAAAAATGGTAATTAGGCTGGGAG
ACCAGGGCTCCAATTCTGTGTATACAGAGGCTCATTTTAATGAATTCTCTGGAGATGGAGAAGGGGAAAT
GTTATTTTGTTTGTTTTTCCTAACATGGTGGCAGAGGCATTCGAGAAGTAGACAAGATCCCCAGGGTCTT
TGGGGATTCAGCTTACCTCACCCCAAGTTGTGCCCCTGCACAGCAATCACTCCCCACCACCCCTTCAGAC
ATCCTTCCAGCAGTACAACCTGGCAGAGCTGTCTTGCCTTGATTGGCTGAGGTGCCCAGACAGTTCCTAG
AATGGAATCTACCCCTCCTTCCCTCTTTCCATCCCACACTTTCTGTCTCTGGCCCCACTACTCCCCTCTC
GTTCACTCATTCTGACAAAAGATGTTTGTCAAATGCCTACTCTATGTTTGGAGAGGCAGAAGAAAGTGGC
CTGGCCACAGAATCTGGCCCTGCCTTCAGCCAGGGCATGAAGGACAGGGAGGGGGCGTTTAACCCTATCC
TCAGTGACAGCTGGTGAGTTCTTTCTCGGTCTAAAACTGAACATGCAGGAGACCCTGAGGGGCTGGCATG
AGGACCTGGGTGTGTGGAGCTGGGTTCTACAGACCTCTCAAGACTTTTTGTCCACCTGGTAGGCCCCGTG
AGATGCCTCAGGGGAGCAGGTCTTCTACCTCCTCTTGCCTCACACTTACCTCCTCACCCCCAAACACACA
TACACACATGTGTTCTCATGACACATGTACTCTCTGGCCTCTCAACACCCTGCCCATCACCTCATCTAAA
TCCACTCACTTGAAGAAGTAAAAAAACAAGAGTCACAGCCAGATGAGATTCATGTCTGCAGTGGTTCTCT
CATATAAAACCCCAACTAAGTAATTTTCAAAATTTATGATAGAAAAAAACCTTCCAGTATGTGCATTGTG
ATGAGACTTGGCTGTCTTTGGGGCCAGCCCCCTTCCCCTTCTGCAGGCCTGGTCTGTGAGCCGCCTGTGT
GAAACCTGTACTCATGCCACCATGGAAGGGTTCAGCCTCTGCAGGTGCTGCTGGGGATGGAGGGGGAGGA
TTTGGTGGTAGCAGCACTCTTCTCCGGTCTCTCATGGGCCCTCTATCACTCCTGGCTGCCTGTTAGCATC
ACCTTTTGCGAGATTCACGGGCTCGGGTATTAGCATGCTTAAAGCCTCAGCAGTGATTGTGATGCACTTA
GAGCCACTGGCTTGTCCCTGGGCCTCACACCATGGCACAAATTAGGAAACCACTGACTTAACGGGCTGTC
CCTGGGAGCAGGTCCCTGGCCTGCCAGAGCTCATCCAGCTGCTCTCAGGAGGACACACTCTCAGAGTGGA
GGAGCATTTCTGGAGGCCTGTGAAGCTCTGCCTGCGCTGGATCCTTTCCTGGGTCCCACAGAGCCTGGGA
ACAGAGTGGCTGCCTCCTTGCCCTCCCAGGCCAAGGGGTTGCCTGAGATGCTCTGGCCTTGCAGGTGATC
CTCACTGCATTCCTGCCGCTCCGCCCTGAGGCTTTCCTCTCTCTTCTCCTGGGGCTGCCTTAACAAATTG
CCGCAGAGTGCCTTAAAACAACAGAAGTTTATTTTCTTACTGTTCTGGAGGTTGAAGTCCAAAATCAAGG
CATGGAGGGCCTTGTTCTCAGAAGGCTCTGGGAGAGAATCCCCCCAGCTTCCAGTGCCTCCGGGCCTTCC
TCGGCTAGTGGCTGTATCACTCCACTCTCCGCCTCTGTCTTCAGCAGGCCTCCTGTCTCTCTGTGTCCTC
ACCTCTTCTTAAGAGGACACCAGTCACTGGATTTAAGGACCCACCCTAAATTCTGGATGATTTTTTCTCT
CAAGATCCTTAATTAATCGTATGTCCAAAACTGCAATTTTCAAATAAGCTCATATTCTGAAGTTCCAAGT
GGGCATGAATTTGGGGATGGATACCCTTCAACTCTCTCCAAAATCCTAGTACCTCCCAGTCTGATCCCAA
TGGGGACTGGACCTTCTGGCCTATTCCTCAGCTTGGGCACCTCCGCACCCCCCAGCATAGCCCCTGCAGG
CTCCGAGCCCCTCCGGCCCTTCACGTGCCTCCTGCCCAGCATCTCGACCTGGTGAGGCCATTTCCCCCGG
GATCCTTTCTTGGCCACATGTCTTTTTATTCTCACAAGGTGTCTCCAGATCATTTCTTTGTGTAACATTT
TGTACGTTGATTTAATGAAATAAATATGAGTGTTTCCTGGGGCCTGGCCATTGTGTCTGCAGGCCACTTA
TCTGGCTGCTCAGCCCATAAATCAGCACCTGCCCCTCCCCAGTGATCTTGCCCTTCTGAGAGGAGCTGAG
CTCCACACACAGCACATCCCAGCTGCTTAGGGAGGGGACAAACAGGAGAGGTCACTGTGCATGGAAGGCT
ATGCTTTATTCAACATGGTATTTGGTTCTCTGCTATCTCTTTCCAGGTCAGCACATTCTCAGGTGGCCCA
GGGGGTTGCCAGCCTTGGCAGCATGACCTTAACAAACAGAGACTCGGGCCTAAAAAGCCCTCGCACTGCC
TTTGCTGGTGTAGGTGAGGTAGGACCCAATGATGAAAACAGACCTGAGTGGGGATCAAGCCAGAAGGTTC
CCCGTGGACACACTCACACACTCATGGACACTGGACCCTGACACCCACCCCCAACCCAGGAATAGAAAAG
GGGCGTGAAAAGAGACACCTGTAGGACTTCCGGCAAGATTCTGAGGTTACTGAGCACTTTTTTCTTGGTG
TAATTTTCCATCATCTAGACTTTAAGACCTACATGAGCTACGGTTTAAATATAAATTTACAGCTGAGGGT
GGGGGTTCACACCTGTAATCCCAGCACTTCGGGAGGCCAAGGCCAGAGGATTGCTTGAGGCCAGGAATTC
AAGACCAGTCTGGTCAAGATAACGAAACCCTGTTATTATAAATTTTTTAAAAATTAGTTGTATATAGTGG
CATGAGCCTGTGGTCCCAGCTACTCAGGAGGCTGAGATGGGAGGATTGCTTGAGCCTGTGAGGTCAAGGC
TGCAGTGAGCCATGATCATGCCACTGCACTCCAGCCTGGGTCACAGAGCAAGACCCTATTTCAATCAGTC
AGTCAATTAATAAATACATACATACATACATATTTGCTACATTCCTACCTCTAAAGTAGGCCACTGGTCT
AGATGATACAGGGGTTAAATTCCATCTGATTAATTTCCAGGTTAAAAAAATGCAGTACTAAGACGCTGAA
AAGCTCGGCATGTTGGAGTGGAGGGCAGTAGTGGGGCCTGTGAGTGGGCTGTTGGCAGGGCTGCATTTTG
ACTTTCATGGGCCTCAGGCATTTCGCTTCATGGGCCCTAACATGGGCTGAATTGTGTCTCCTCAAAATTC
CTGCATCGAAGTTCTGGTCCCAAGTATCGCAGCATGGGACTGTAGATAAAGATAGGGTCTCTACAGAAGT
AAAATGAGATTATTAGGGTGGCCCTAGTTCAATATGACTGGTGTCCTTATAAGAAGAGGAGATGAGGACA
TTGACATTCACAGAGAAAGACAATGTGAGGACACAGGAAGGGACAGCCATCTGTAAGCCAGGGAGGGCCT
CTCAAGAAGCCAATTGTGCCTACACCTCAGTCTCAGACTCCAGCCTCCAGAGCTGTGAGGAGATGACTGT
AGTTGAAGCCATCCAGTCTGTGGTACTTTGTTATGGCAGGCCTAGGAAACAAATACAGGTCTCTTCCTTT
AAAAACATGTTAAGAATTATATTTTTACAGCTACATTGGTATAGAGATGAATGTAATTCAGGCTGGATTC
ATTATAATTATAGTCATTTCTTCTCCTGATTTTAAGAGAAATTAAAATGGAAACATTTTCCTTGGCCCTT
AAGTGTCTTACGGGCACTGGGCTCTGTGCCCCTCGTGCCTGGTGGGTGAGTTGGCCCCAGTGACTGGGAC
TGTGGAAGGCCCGAGGGCGGGTTGGGATCCCTGGGTAGGAAGGGTGCGCCATGGAGGGTTCCTGACCCAG
ACACTGATGGGATCAAAGATAAGGAGTTTGTGGGAGGCAGGCCTGGAGGGGCTGGGCCGGGACCAGGGGT
GGCAGCTGAGGCGGGGCACACAGACTCAGATACAGCTGTGTGTGGAAGGGCAAGGAGGGCAGGGGCAAGG
GAGGAACCATGCAGGGCTGTGTGGGAGGTGAGCGAGGGCCCTAGGAGAGATGGCACAGGAAGTGGAAGGG
GCCAACGAACAGTGTGTCTGTGGGTTACATAGAGAAGAGGAAGAGGCCTGAATGCGCCCCAGGTTTCTGT
CTAGAAATGCTGGAAGAATATCAGTGCCATCAATACAAAGAAAAAGGAAGAGTCCAGTGGGAGGTAATGA
TCAAACAGCCAAAATGCTTGCAAATGGCTGTTCTATCAGGCCAGCCTGTGAGAGACTGGGCAAATCTAGA
TTCCTAAAGCCCTAAAAATGGGTGCGTTTTCTCTAATGGAATGTAGAACGGAGGCAGAGTGCCAGAAGGG
ATCTTTTACCCACAGAATCTAAAATGCCGCACGTCCCCAGCACCTGGGGGAGATGGGACAGGATCTTCTG
CCCTGGTAGAGGTGGTCAGAGTGAGGGCACACGCCAAGGAGGATTCAGAATGTCCCCTATGCCTCAGTCC
TTTTTTTTTTTTTTTTTTTTTTCCCTTCAAAAAGAGGCCTAGGTTTAAGCCCCAGGTTCAAGTCCCACTT
CGCTGGGAGACCCTGGCCGAGCTTTTTAGTTTCTTAGTATTACTCTTTTTTTTAAGCTGGAAATTATTCA
GATGGAATTTAGCCCGTCTATCACCTAAACCAGTGGCCTTAAACTTTGGAAGTAGGAATGTAATTAATTT
GTATTTGAACTATGGCTCATATAGGTATTAAAGTCGAGGTGATGGAAAATTATGGGCCAAAACCACTGTT
GATAGAAATGTGCATTACTGTACAGAAACCCTGCAACAAACCAAGATAATAGTGATAAAGTGAAACAAAT
GACAACTTTCCACAGTGTCCCGACCATCCCTGCATGTGCACTTGTGCTGTGCTTTGCAGCTCTGCCAAAT
GATGCTCAACCCCATAGACTTCGGGCAGAGCACTGGACTGCAGTCCTCGGTTAACTCTAGCAACATGAGG
ATATTTTTTATATTAATAGAGTAGAGTTTCATTTTTTGCAGACATTATGTTCTGGGGTCACAATATGAGG
TGACACTCTTATGTCGTGAAATCTGAATGCCAATCACTCTGGCAAGATGACCCTGCCTCAGAGACCCACT
GGGGCACTGCAAGTCATCCAGGTGTTCCCTCTGAACATTGACCCGGCAGAAGATACTCCAGGCGTTGGAC
TTTTGAGGGCATCTAAGAACTTACTTATGGTAACTACAAAATGAATTAGGAAAGATTACTAAAGCCTGGA
GCAGAGAACTGCATCCTGAGTCCATTCCACTCATTAAATGTATCAGGAGCTACTGGTGGGCTCACCCAGT
ACCCATTGGCAACCTCCTACTCCCTCGCTTGATTAGAGACTGTAAAAGCTAAATTTGGCTTTCCCAGCCT
CTCTAGAGGCTAAGAGTAATGATATGACTCAATTCTGGCCAATAAGACACAAGCAGCAGTCTGCTTGGGG
GGAAGGATTCCAGGGAAGCTCTTGCTTGCTTTTTCTGCCTTGAATGTGGATGTGATGTCTGGAGCAGCAA
CAGACAGTCTGTACAAAGATGAGAACCCATGGGCCACGACAGCAGAACAGAATAAGCAATAGTCCAGGGG
CCTGGTGTACCACCACTCAGTGTGCCAACCACACTGCCCAGGCTCTAACATCCTTTCTTTCTCTCCCTCC
TCTCCTATGTCCTCCCGTCCACTTCTTTGTCAGCACATGCAAATTAGAAATGTAGTCCCCTCCTAGTACA
GAAAGCTGATGAACAGTGGCATAAATGAAGGAGAGTTTTAATTTTGTAGGTGCAAGAAGTCATGTGGTTG
GCACACTGAGTGGTGCTACCATCAGGCCCCTGGACTATTGCTTTTTCCATTCTGCCGTCGTGGCCTGTGG
GTTCTCATCTTCGTGCGTGCTGGCTGTTGCTGCTCCAGACATCACATCCACATTCAAGGCAGAAAAAGCA
AGAGCTTCCCTGGAGTTCCTCCCCGCTCCAGCAGACTGCTTCCTGTGTCTTATTGGCCAGAATTGAGTCA
CACCATTACCGTTAGCCTCTAGAGAGGCTGGGAAAGCCAAATTTAGCTTTTACAGTCTAAAGTAGTCAAG
CAAGGGAGTAGGAGGTTGCCAATGGGTGCTGGGTGGGCCCACCAGTAGCTCCTGATACATTTAATAAGTG
GAATGGACTCAGGATGCAGTTCTCTGCTCCAGGCTTTAGTAATCTCTCCTAATTCATTTTGTAGGTTACC
ATAAGTAAGTTCTTAGATGCCCTCAAAAGTCCGACACCTGGAGTATCTTTTGCTATAAGAGTTGAGGGGA
GGGCTGGCACACATGAATCCATTTGTCACCCTTTGGGAGGCCTCTCAGGGAGCTTCAGGCCACACGACCT
TGCACATTATGGGTAGCTTTTGAAGGGACCCACTGATATTGATGAAATTTAACCTTATTTAAAATTTGAA
GGTGTATGTTGTTTTCAGACCACAGTCAGGAAATAAAAGAGCAATCACATATTTAAATTGGGTGGTGACT
AAAGTACAAGCCACTCTCCATTGGGAGAAAAGTGGCCTCCAGGACCATACAGACTTTGAAACTGAGGTAT
CTGGAAGAATGCAAGTACCCGTACTGTGGCCTTCTGAGCCAACATCCCCTCCCCCAGGGCGCATTACTGC
GCTTCTGGCAAGGATATGCCCAGCCTATTTGCCAGAGACTGCTGTCTCATCTCTCCAAAGCACCCACTTG
CCAGTCTGGAACCTGAAGTTTCAGCAGGCTCATTTGCAAGCTAATTTACATGACTTTACCCTGATACTTT
CATTTAATGGTTAGTGGTGCAGAGCACAAACAGCAACTTCATTGTCAAAGGCTTGTCATGCCCAGCTGCT
TTCTGCCACTTATGGATGCTTCCCCAGCCCTGGGCCCAGGCCTGGCCAAGGCTAAAAGAAGACGCACGTA
ACATGCCTTTTCATTATTTCTTTTCCTGAGAAGAGAATCTTGAGTCAATTTATGCAAATTACCATGCAGA
TTTCCATTGCAGACTTTATTACTGTGTCTTGTCAGAATGTGGAAACAAAATTGTATTTCATAATAAATGC
ACATAGCAAAGAAGACTTCAACAAGTGCCTCAGGAAACGTCTCTTTCCTCCGGGATCAGCTGGGCCAATG
GAAGCTTCTGTGCAGAAGGAAGCATGTAATGTACTGTCATTCTGCACTGTATGTCTGAACCCCTGTAGAT
ACTGTTTAATACCATGTTCAGAAGAAGATAAATGTGGATTCAGCTATGAAATCCATGCTCATAAGATTCC
TCACATTTTGTATAAAATGAAGGGCCTGCCATTTCTCATATACAGTGCTGTTTGTAAGCACAAAGATATT
AAAGGAATAAATAAGCATTGCCAGGTTTGGAGCAATCTCATCCCAGCAGAGTGCAGCTCAGCCTTCTGAG
AGGACATGGTGCAGTGCAGGAGGTGTGCCCAGCACAGCCGAGGAGGGAGAGGAGGGTCATAGGGATCGTG
GACTGGCTGGCAGGAAACTCCCCAAGGTCAGACCTGCTGCTCATGCTGTGTTGATATCCTGCGTTTACAT
CCCTGTAAAATAGTTACCAGCAAAAATAATTTGCTTTGTACCTCACAAGAGAAAATGAAGTTCAGAGAAA
GAATGCTTTGACCAAGACTCCATGGCAAGCTAGAAGAGGGAAGTAGTCTCCTTTAGAACCCGTCTCTCTG
AAGGTCAGCCTTTCATGAGGCTCTGGGAATACTCGGTTATTGAGATGTTTGTAGGGATTGCAACCACAAA
GGTTCCTAGGGCAAAGAAGCTGGGGAAACATTGGGTTCGTGTTTCTTCAGTGCAGAACTCATCAGAGCCT
TTTATTTGCCAGTGTGCCTTATGAGAGGCTTTACTAAAGAGGCTATCTACTCAGGGCTAGAATATGTAGC
CTTTCCCAGAAATACTTGATCACCATTTACTTTTGTTCCAAAGATGGTCTCATGGGGCTGTGTTTCACAT
AACCACACTGAGAAACATTGCTGGAGAGTCTCCATGGGGTGGGTTCTACAGCTTCTCTTAAACCTCTAGG
TCAGTCGGTCACTAATAACAATTCATAATCTTTGTTTACGCAGCATATCACAGAGTATGCATTATCTTCA
CTTTCTCTAACTCATTTGATCCAACAGTCCTTAAAGGAAGCTGTGACTTTTCATGTATTAGAGATGAGGA
AGCCAAGGGCCAGGGAAGCTAAGCCATTTTTCCAGGACACTCCCAATCCAGTGTTGGTGTTGGCAGAGCA
CTCAGACCTTGCCAACACTGACAGAGGTCACCAAAAACAGACTGGGGCTCCCTTGGCATATTACCCCATC
CAAGTATGTTTGAGTGGGGCACTCTGCCAGCTGCATGGCCATTCCCCTGGGGCAGGGTGCATGGTAGCTC
CTGTGGGGCTGGCTATGGTCTGTCCTGGGGAGCATTGACAGGCATTCTAGATTTGCCGGCAGACATATGC
TCCCCATCCCAAGGGCCTGTGGAGCGTCCTCTTCACCTCAGCTGCCTGGCATTGCTGGGCTATGTGCATA
TCATGGATCTGGGCTTTATCTTCAAAGAAGAAATGACTCCCTCCCTACCCCCACACTCTCATACCCACAT
ACACATACTTCTACATGCAAATAAATACATAATTACATACACATGTGTATCCCCACTCATCCACGTGTCT
GCTCACACACACTCATACCCACCTTCCCATCAGTACACACTGTGGCCTGTGGTGACCTTTGAGATGAAAT
GTACCCTGTGGCAGGTCACAGGAAATCACCAAGGTTGCTGGGGCCTTTTTCAGGAGGATATGGCCTACCT
GGGTGGCTAGTTGTTCTTTTTGTCTGATGTCCCCATATCCCTCCAAAGTAGGAGCCACAGATATGACTGT
ACTACAGATGCAGAGGAGGAAAAACTAGCATCGGAAAGGAAAAGAGGGTAGGGGAGATAGCTATAGCAGA
AATACTGCATGGAGCTCAGCATTTACCTTTGAGTTTCCTGGTGGCTGGAGGGAAAGGAAATACCATGCAT
GGCTCCCACATACTAGTGGCAAAATGCTCCCAGGCTTGCATTTTGAGCCAGATATTGCTAACACTTGAAC
AAGGTCACGCAGTAAGGCAGGGAGAGTGGTCAGGTTTAGAATCAAGGATTTGCCAGGCATGGTGACTCAC
GCCTGTAATCCCAGCTCTTTGGGAGGCTGAAGCGGAAGGATCACTTGTGCCCTGGAGTTCGAGACCAGCC
TGGGCAACATGGTGAGATCCCCATCTCTACAAAAGAAAATTGTTTTTTTAATTAGCTGGGCGTGGTGGCA
CACTCCTGTAGTTCCAACTACTTGGGAGGCTGAGGCAGGAGAATTGCTTGTGCCAAGGAGGTCGAGGCTA
CAGGGATCTGTGTTCATGCCACTGCACTCCAGCCTGGGCAACAGAGTGAGACCCTGTCTCAAAAAAAAAA
AAAGAAAAAGAAAAAAATCAGGGTTTCTCTGCCCCAGCTTACAAAGAGGTATATGTAATGGCTTGGCAGG
TAGGGCATCTAGTGACAGTGTTACCAGCAAGATAGAGACTTTCCTCACATGATGATTTGTGATACAGACC
CTCACAGACCTCCAAAGTGCAATGCCAGCATAATCTGGCTTACTTGCTGCTAAAACCATCCTGGAGAGTG
TGGACAAAGCAGCCTAGGCCCGTGGTCTCTGAGGATCTGATTTGGCACAAGTCTGAGACTAGAGTACCCA
GCAGAACAGTGGTAGACACAGCTCTTAGACAGAGCCCCGCAGCCCCGGGAATCCCAGGAGCACCATTGGC
TTTGATGCTTGGCAATCTTGTATTGGCTGGTAAGCCCAGCTGCCTCGCAAAACCACGCCTGGACCACGCT
CATTCCCTGTGACGAGGAGAATGCCCATCAGTGTAGATCTAACTTTGTGGCTCTCCATGGAGGTGGCATG
AATGACACCCAAGTGTGGTCGTTTCTCATTAGTTCTAGTGGTGTGGTTGCAGTTGCTTCCAGAGAAGCAG
CACCTTGGGCCATCCTTTAGTGGCTACCCTAGCTGGGATGTGCTCCAAAGGAGTCAGCCCATGAAGGTCG
TCTTTTATTTCATCACCCAAAGCCCATGATTTATCATCAGAGAAGATGCAAATTAGTTCCTGTGACCTCG
CTGACTTGCTATGGGACCTCAGGCAAGTCACTTCACTTCTGTGTGGCTCTGGCTTATCTGCAAAGTGTAG
TTAGTGTTCCCAGTCGTGCAAGCCACTCCCCCCTTTTCTTGTTAACAGTACCTTATCTTAGTCACCCAGC
CTCTCTACATCTTTCACACCCATGGAGTCCTGCCTCTGCTTCTTGATATCACTCATGTGTCCTCCTTCCT
TCTCACTCTGCTCTTCTCTGCCCACACCTCTTTGCCTGTCTAGGCCTTTGCAGCAGGTTCCGTTTTGTCT
CCCTTGCCTTTATCCCATTCACTATCCTTCAACCAGGGTTATCTTTCTAAAGCCCCAGCTACATGGAAGT
CCCTCTTCTTTGGTCGTTTATCAGTTCTTTGGCTCCATCCTGTTCTCTACAGGCAATTCACAGTCCATTG
GTCCTGAATCATTTTTGTTTTTCTGAAGAAACTGTGTGGCTTCACACCTCAGGGTGTACTGAAGCCATCA
CTTTTATCTAAAACAAACAATGTCCTGCATTGACCCATGCATGTATTTACCATTTCTGATGCTCACAATT
TCTTCCAGAGAATTCTAGTTTCCATTAGTATATTTTCCTGTCAGCCTGAACAGCTTCCTTTAACATTTCT
TGTAGTGAAGTTCTGCTTGCCACAAATTCTCTTTGTTTTTGTTTATCTAAAAATATTTTTATTTCTTCAT
ACTTTTGAAGAGTATTTTTGCTGGATTTAGAATTCTGTTTCCACGGTTTTTTTTTTTTTTCTTGAACACT
TTTATTGTGTCATTCCAGTGTTTTCTAGCTTCCATTTTTTTCCAGTATGGCAGCCGTCATCCCTGTTATT
TCCTTGCGTGAGGTGTGTCATTTTCCTCTGGCTACTGTCAAGATTTTTCTTTATCTTTGATTTTTAGCAG
TTAGACTCTGTTATGAATAGACATGTGATTTTGTTTCTTTATTTTTTTAATGTAATGCTTGGAGTTGGAT
GAGTTTTTGAATCTGTAAATGTGTCTCATTTTGCACTCAGAAGACTTTTCACCATTATTTCTTTAGTTTT
TTTGTCCAATTCTCCATCTCCTCTACTTCTGGGACTCCAGTTAAACATATGTTAAGCTGTATGAGGCTCT
GTTTATGAAATTTTTCTCAATTTTTTTTCTCTTTATGCTTCCTTTTGGATAATTTATATGATCTATTTTC
AAGTTCACTGACTATTTCATTTCTCTTTTTGAATCTGTTGTTTCAGATGTATTTTTCAGTTCTAAGTTTG
TATTTGGTTCTTTACTTAGAGTTTTAGTTTCTCTGTTGAGATTTTTAAATCTGCGCCTTTATTATGAGCT
TATTTTTATATTTGAGCATAGTTATATGAACTATTTTTAAATGCTTGGCTATTTTCAACATCTGGGTACC
TCAGAGTTAGTCTCCATTTAATCTCTTTTTTCTTGAATATGGCTGACATTTTCTTATTCATATGACTAGT
AATTTTGGGTTATACATTATGTTAAAACTGTGGATTCTGTTTGTTCCTGTGAAGAGTACTCTTGGTTTTA
GTGGGCAGCTAAATTGTCTGGACCCAAATTCTCAATTCTCCCCCCCGTGGTAGGCAGCAACTGAAAGTTA
TGTTCAGATTTTTTTGTTTGCCTGTTTTATTTTGTTTTCAGTCTTAGGTAGGCTGCTTAGGGTCTGCTTG
GGTCAGTCAGAGATTTGGGTAACATTTATATGGAGAATTTGGGGTCACCTCTCTGTATCTATGTCACTTC
CAGGGTTTTCCACACTTTCTGGCTGCTGTAGTAACTTTGCAGATTGTTCTCTAATTTCTCAACCAGTAAG
ACTGTTTGTGCCTTATTTGAGGCAAATAGATGATGTCAAAGAGCTAGAAATTTTCTGGTAACTGAGGCCC
AGCCAAGGCCCTCTTAAAAGTTCTTGTTTCTATTCTGTCCTTAGAGAGCTGACTTTTCTCTTCTGGGAAA
TGAACTTCACCTTAAGTGGGCTCTGCTGAACTATTATGGCTGATGACCATAAGATTATGACAGTAGGTAG
CCAGATAGTTTGTGACCCCCAGAAATATTCAGTGACAGCTTCAGGGTGCATGACAGGTTCAGAAATGAAG
GGATTTCAGGAAGGAGAGGGAGTACAACTGAATAGTCAAGAGCAGGGACTTTGCATTCAGGGAAACTGAT
TCTGACACTACTGACTGTGTGACCTTAGTTCTGAGTAAGCCTTGCTTTCTTCTGTGAATAGCACCTTCTC
ATGCATAAGCATCTCCCTCTTGTCTCTCACCCTGTACAGGGCATCTGCCACTCCTGGACAGCACAACCTC
CCCAGACTCTGATTACAACAGAAGGCAGTTTGCAAACTTTACTGCACAAATGAGTTATCTGAGGGCTTGT
CTGGAATTCTGATTCCCAGAGATTGTGATTCTGTCTGTCTGAGTGGAGCTCAGGAATCAACATGTTTAAC
AAACTCCTGATGTGATTCTGATGCACATGACTCGTGGGCTACATATTGAGAATCATTGAATCACTGGCCA
GTGGGAGAGATTGATTAGTATGAGATCTTTCCAGTTTAACAGAGAAAGTGTTAAATGAGGGGCCCTGGAC
CAACCTGAGGGCTCAGAAAATGATTTGTAGAGGAGGTGAATCTTGAATTCTGAATAAGTATAAACCATGT
GGAAGAGCATTCCAGAAAAGGGGACATTATTAGAAAAGGCCCAGACATACAAAGCAACTTGTTTATGAGG
AGTTCTGTACAGAATATTATTTCTAAAATATAAAGTGGAGGCTGGTCAAGGAGGGAATGAGTCCATAGAA
TGCCAGGGGCAGGCCCTGAACAGCATTATAGGCCAGCTTGAAGTGTTTGAGCTATCTCTTAGAGGCAGTA
GATCATATTAATATGATCATATTAATATTTCAGAAAAATAACCCTGTGCAATGGTTTCAGTGTCACCACC
AAAACTCATATTGAAATTGAATTCCCATTGTGACGGTATTAAGAGGTGATCCTAGAAGGTGCAGCTGCTT
TAAAAAAAAAAAAAAGTGGAGCCTTTAAGAGGTGATTAGGTAATGAGGGCTTCACCCTCATGAATGGATT
AATGCAGTTATTGCAGGAGTGGGTGGTACCATCGGAGTTTGGGCCCCTTTTTCTCTCTGTTGTGTGTTCA
CTTTCCCTTCCACCTTCTGACATGGGATGACACAGCACAAAGGCCCTCCCCAGACACCAGTGCCATGTTC
TTAGACTTCGCAGCTTCCAGAATTGTGATAAGTAAATTTCTTTTTAAAGTAAATTATCCAGTCTGTGATT
CTGTTACAGCAGCAGAAAATGGACTAAGACATCCTGCCTGCTACATGAAAGATTAATTACAGTGGAAAGC
ATGAGGGATAGTCAGAAAGCTGTTGGTGTTATTTAGGTAAAAGAAGTTCAGTGTCTAAACTAGGGCAGTG
GTGTTGGGGATGGAGAAAAAGGGGGCTGGGTGAGAGCCCTTAGGAGGCCAAGCAACCAGTTCAGTATTCA
TTTGATGTGAGGGAACAGACAGCAAGTGAAGAGGAGCAACTCAGGAGGGCTTTCAGGCTTCCAGAGGCGT
GGGCGCCTGGATGGTCAAGGTGCCATTTATTGAAATAGGAAAAATAGCAGAAGCAGGTTTGGAGGAAGAT
GATAGGCTCTTTTTGGAATATACTATATTTAGTCAGCTGTCTAGTGGATAATTAAGTGGGATTTGTAGTA
AGTAGCTGGATGCATAGATTTGGGGCTCAGAACAGGGGTAAGTCCTGGGTTATAGATTGGGAACTGATCA
GTATATCATATTTGAAGCATCGAGAGTAAATGAGATCCCCTCTGAAACAAAACAGTGGAAATGCCAACAT
TTTAGGGAAAGAAAGGAAGAAGATCCTAGAAGAGAACAATAAGGAACCGCAATTAGAGAGGCAGGGAAAG
CCAGAAACAAGTAAAGTCGTGGAAAGCAAAGAAGAAACAAATGCCATAGAAGAGGAATGGTCAGCAGGGT
CAAGTGTGTTGAGTCTGGACTATACAGTGTCCTCATGACTTGATAATCAAATGGCTATTGGTGACCATAG
CAAGAGCTATGGGCAGAGGGACAGAAACCAGACTGACCTAGTTGAGAAATGGGTGGGCATAGAGGAAATG
GATACAGTGGGTTTGAAGTCCTTTTCCAAGAAGCAAGCCCATTGAGAAGAGGTGAGAGATTGGCAGATGG
CTAGAGAGCAACTTAGTGAAAATGAGGGATTTTTCCACTCAAAAGATGTGAGAATCTTGAGAATGTTTCT
TGGATGAAAGGAAAGAGTGAGTAGAGAATGAGAGACTGAGATATAGAAGAGAAAGGAGATGGCTGAGAAA
GCATCATCCAGAGAAGGCCAGTGCCAAGAGGGCCTAGGAAGAGGCTTTCTTTAAACAGGAGAAATAACGA
ATACTTAGAGCCTGGAGGCGTGTGGGGATAAGGCTGGACATATGTAGACAACTTTGCAAATACCTTTTGA
ATCTTGACTGCAGTCTCAGCTGTGGACATTTGACAAGTCGCTTAGCATCTCTTGTCTTCCATTTTCTTAG
CAGTGACACAGAATACAGAAGAGCCTAACTCATCACATAGATTTATTGTCCAGATTAAATTAAGTAATGT
ATTTAAAGCAGTTAGTACAAGAGCTGGGAAATGATAAAGTGCTGAGATGTTGTTACTGTGGATGTCATTG
TTACTGTGATAGGACGCAATCAAGGACAAGTAGCGTGTTTTTATATTTGCCCATAGATTTGCCATTTCTG
GTGCACTTGTATTCCAGCCTTGAAGATTCTAATTTCCATCTACAAAATTTCTCTTTAGCCTAAAGCATCT
CTTATAATGAGTGCATGCTGGTGACAAATTATGTGACTTTCCCTTATGTGAAAATGTTTATGTTTTGTCT
TCATTTTTGAAGAAAATTTTCACCAGCTATAGAATTCTAGGCTGACAGGGTTTTTGTGTGTGTGTCTTGT
TTTATTTTTTAAGCAGTTTAGAGACGTTGTTCCCTTGTCTTCTGTCTTCCATTGTTTCTGATGATAAATC
AACTATAATTCTTACCATTGTTTCCTTGTATGTAATGTGTTGTTCTGTTTTCTGGCTGCTATTACGGTTT
TCTCTCTACTTTTTTATTTTCAGCAATTTGACTATGACATACCTTGGTTTGGTTTTTTACAGATTTATCC
TGTTTTAGGTTCACTGAACTTCTTGGAACTGTGGGTTGATGTTTCTAATTAAATTGGGTGAAGTTGGGGG
CTATTCTCAAGATGTTTTTTGCTTGCATCTGTTTCTACTGTCCTGAGACTCTAATTGCATTTTTTGTTTT
ATTATTTTTTGGGAGACAGAGTCTCACTCTGTTGCCCAGGCTGGAGTGCAGTGGTGCGATCTCAGCTCAC
TGCAACCTCTGCCTCCCAGATTCAAGCGAGTCTCCTGCCTCAGCCTTCTAAGTAACTGGGATTACAGGCG
TCCGCCAAGATACCTGGCTAATTTTTGTATTTTTAGTAGAGATGGGGTTTCACTGTGTTGGCCAGGCTGG
TCTCAAACTCCTGACCTCAGGTGATCTGCCTGCCTAGGCCTCCCAGAGTGCTAGGATTACAGGCATGAGC
CACCATGCCCAGCTTCCAATTGCCATTTATGTCAGCTCACTTGATATTGTCCTAGGAGTGCTTGAAGCTC
TCTTTATTTTTATTCCATCATTTTTCTCTCTGTGCTTCAGTTTGAACAATTTATTTTGGCCTGTCTTCAG
GTTCACTGAGCTTTCCTCTGCAGTGTCCTAGCTACTGTTATTTAAATCCAATGCATTTTTATTTCAGATA
TTATAGTTTTTAGTGTTAAAAATACCATTTTGTTTTGTTTTAGAATTTTCATAAATCTCTTGACAGTCAC
CCATTGTATTAATTTTTAATTTTTTTTTTTACCTATTTATAAGAGTTTTAAAGTCTTTGTCTACTAATTC
AACATTTGGATCATGTAGGGGTCTGTTGAGTTTTTTCCCTTGATTATGGCTTGAATTTTCTGTTTCTTCA
CATTTCTAGTCCTTTTTTATTGTACAGTGGACCTCATGAATAATAAGTTATAGTCTTGGATTTTGTTACC
TTCCTCTGAAGAGTGTTGTTTTATCTTAATAGGCAATTAAATGATGGGCAGTTAATATTTTTTAGGGAAT
GGTTAGTTATCATGTATGGACCTTGTTAAGACAGATGTCTTCATCCATTCAGGCTGCTATAACAAAATAG
ACTGGGTAACGTATAAGCAATTGACATTTATTGCTCACAGTTCAGGAGTCTGGGATTTCCTTCCAAGATC
AAGGCTCCAGCAGATTCCGCATCTGGTGAGGACTTGCTCTCTGCTTCAAAAATGTCACCTTTATGCTGTG
TCCTCAGTGGTGGAAGGGACGAACACTGCGTCCTCACATGGCAGAGGGGAGCCAGGGTGCTCCCTTAAGC
CTCTTTTATAAGGGTACTAATCCCATTCAAGAGGTCAGAGTCCTCATGACTTAATCCTTTCCCCAAAGGA
TGTGCCTTTTAATACTATCACATTGAGGATTAAGTTTCAGCATTTGAAGTTGAGGTTACAGGGGACACAT
TCAGACCACAGCGATAGATTTATTTTGGTTTTCTCTGAGTCCTCTGGTGTAACCCTCAGTCCTGAGATGA
TCTTGTTCCTCCCAAAGCCAGCCTTTCTGGTGTTTCAGTGGAGTCTGAGGTGTTTTTGAAGTTCTTTTAA
TTTGGTGGGACTGGGGAGTTTCGTCAGGAGGCAAATTGATTTAATGTAGAATCTGCCTAGTTTTCTTCCA
TTCCTTGAGCGGTTGTAACTCCTACCTACTTTTTTTTTTTTCTTTTGAGCCAGGGTCTTGCTCTGTTGAC
CAGCCTGGAGTACAGTAGTGTCATCATGGCTCACTGCATCCTCAGCCCCACCAGCCTCAAGCGATTCTTC
CCCTCAGCCTCACAAGTAGCTGGGACTACAGGCAGGCACCACCACACCTGATTAATTTTCATTTTTTATT
TTTTGTAGAGGCAAAGTCTCACTATGTTACCTAGGCTGCTTTCAAACTCCTGGGCTCAAGTGATCCTCCT
GCCTTGGTCTTTCAAAGTGCTGGGATTACAGGCATGAGCCACTGCACCCTGCCTCGTCCTGCTTTTGATC
ATTCTTCAGTGCTTGCAAATTGTTATTTCTTGTATTTTTCCAGATATTAAAATAGTTTTCTGTTGGAGGG
TTTGTTTGATACAAGCCACTCAGCCATTATTGAAAGCAAAACTTAGGAATATGTTTTTGAGCAGAGCTGA
GGGCTCAGTTACACCCAGAGACAATGCCTATGCTTTGGCGCCATCTCCACAGCTATAGAAGTTCTTCAGT
GGCTCAGTGGCCTGGTCAGATGGAGAGTGTGGCTAGAACAGGTCTGAGAGAGTGGACAGTTAAGGGACCA
GAGGTTTCTCTGTTGGGGAAGCTGGAAGCAGGAATTAAGAGAGAGCTGGAGAAGATGGGATGTTGGCCTT
GTTCAGGTTCCTCGTTGCTGCCTCTGGTTAGTCTGTTATGGGATGTGAGAGAAAGTATGTGGATGTTCAG
TAATGGTGTTTATGATTATGGAGGCCACTGGGTTGAGCTTTGCTGGGACTTCATCCAAGTGGCACAATTT
CTTGTTTCATTTGGTGACTTCTCTGCCCTTTGTGGAAGCAGAAGTGCCTTGTGACATGTGAGGTGTGATG
GGGTTGTCATCATTACAGTTATCAGTAGCATTACCATCAGGTGCCTACCTTTGAGAGATACGTTTCTCCA
TGGTTCTTTGCTTGTTAATTAGGCTATGTTAACAGAAACCCAAAATAACAGTGACTTAAACAAAATAGAA
CAATCTGAGCAGTCCAGCGCTGCTGAAGTAGCTCCTCAGGGTCAGGGAGTCAGCCTTCGTTGATGTCATC
GCTCCTCTGTCATCCTCATCTGGATGGGCCACTATGACTCATCCCCACCGGACAGTGGGAAAGGGAGCAG
CTGTGGGTACTCCCTACCCACAGAGGACCTGACCCCAAAGATGCCTACATCACGTTCACATCCCATTAAC
CAGAATGTAGCCGCCCAGCTGCAAAGAAGGCTGGGAAATATGTTCTTTATTTGGGGCAGCCCTGAGTCCA
TCTGCACACCCAGAATTCTGTTACTATAGAAAGGGAAACTACATACTGGGGACAACCAGCAGTCCCTGCC
ACCAGTCCTAAGCTCATGCACCTGGTTTTAGTTCAAAGGTGACTCTACAAAAGGAGCATGTCACTCCTGG
GGAGCCTGTTGAGCATTGAGGAGTTGAGAGACTTCTACCTGCTATGCACATTTCTCCATAAGGGAATCAG
AAGACAAGGATTGTCAATCTAACATACACCTGTGAAGGAAAAAGTCTGCATCCACAAGCACTGGTCATTG
CAGATTTGTGCCTCGGTGGCTACACTATGCGAAGCTGTGGTAGAAAGAGCCCAGGTGCTGCCAAGTTTGC
AGCTCTGATCTTCCCAGCCCTGCTACTGATGAGTGAAGTCACACTAGGCATGTCCTTTAACCCTCTGGTC
TGTTCTTTCATCTCCAAAGTAAGGGATTGATTGGATTGTATCTAAGGTGTTTTGTCTAGTGCCCACATTC
TAGCATCGTATGCTCCAGAGCGTAGAATGACCCCCCATATCTTTGTGTTATGAGGCAAGCTTCCAGGAGG
GTGCAAGCCAGTCTCAGTCCTTTGTGGAACGAGTTGGGAGTGTGACTGTGTAAACATGCACACTTGTGCA
CACACATTGGTGCATACCTTTGCATTCAGCCTGGGGGGACTGGAGATATGACCTTTTCTATTTTCTTGGG
CTTCCTTTGGTTGTGTTTTTTGCAGTCAAAAATCATGGCATTCCACACAATTAAACTTATTTATTAAACT
TTATTTCAAAGGACTTTTTCTAATTTTAGTACTGTTATATGTTCATTGTGGAAAGTAGAGAAAAGTGTAA
TGAAGGAGTGTAATGAAGGAAAGAAAATGCTCTGTCACTATTCAGACCGTGATGCTATTAACATGTTAGC
AGATTCCCTTCACTTCAGTGTCTAAGAAAACATGAAAAATACTCATAGATCTATTTGAGTTAATCTAATT
GGGATCACTCGCCACATGATGTTTTATAACCTGTTTTTTGTTTATTTGTTTTTTTAACTGGATAACCGTT
TCCCCAAATCCTTAAATATTTTTTCAAGAATGTGATTGTTAACAGCTGCCAAGGGTTCTAATCACATGGA
TATACCACACTATGCTTATTTAGTGCCGTGGACATTCAGGCTGTGTCCAAGTCTTTGTTATTATTGATTA
CATAGCAGTGATATCTCCATTTTACATTGACAGCATCTCAGTCCTACTGACAGGTACCGGGTGCCTGTGA
GGCCCAGAGAGGTTGGTTGCCTGCCCAAGGCCACACAGCAAGTGAGTGGTGGGTCCTGGCCATCTGGCCA
CAAGGCTGGCTGCCTCTCACAGGTGCCAGGGCAGAAGAGGCATAGGTGTGGCACAGGGGAGCTGGTGACA
GCTTCCAATGTTTTGGCTTGGGGAGAAAAGAGCCAGTGGGGCCTCCCAGCGAGAGGCCACTCTGGGTGGT
CTGCAGTGCCCGCACCTGAGAAAAGCCATGCTGCTGTTTGCACCCACACAGCAGCAAGCTGCCTGCTCCT
GCCCCCTGCCTCCCACCCTCCCGAGGTCAGTGTTGAGCCCAGGCCCATGCTTTAGGGCAGAGGGAAGAGG
CTTTGGAAGCTGCAGGTTAGGCCATGAAGGGAACAAGGCAGGGAGTCCAGGTGGGGAGGGCAGGATATGT
GCGTTGAGTGGCCTGAGTGGCCTTGCTCCTGGGGAGGCCTCCTCACCACTGCGCTCTCACTGCTTCTGAA
GCACTTGCCCTCTCCAGAGAAGATCTCACCCTATAAAGGCCACCCTCATCTTTCCTCAGCTTGATCCCTC
AGTGAGAGAGCCGCTGAGTGGTAGCAGGGAGGGGCAGGGAGGACAGAGCATGGCCACATTAGACCTGACA
TGAGCGTCCCCTCTTGCTGGTTATGCCACCACCTGTGGTCGCTAGGCTTTTGCCCCTTTGCATGAGACCT
TTTGATTTTGACCGTTCTGTGCTTTTCTCTAACAGGCAACCATTGGGATTGACTTCTTGTCAAAAACCAT
GTACTTGGAGGACCGCACGGTGAGCACCTAATATCAGGACTGGGAGGGGCAGGGAGGGTTCCTTTGGGCT
TATCCCCTAGGACCCTTAGCTGGGCACCACTGAGAGCACCTGCCGACACTAGGGGCTGGAGCTGGTGATG
CCAGCATTGCAGAGCCCGGCTCTCCCAGTCCCCCGGACCAGGAGGACCTCACTGCCCCACTGACCCCTCT
GCCCATGGTCCTGCCCTCCCTGAGACAGGTGCGACTGCAGCTCTGGGACACAGCTGGTCAGGAGAGGTTC
CGCAGCCTGATCCCCAGCTACATCCGGGACTCCACGGTGGCTGTGGTGGTGTACGACATCACAAGTGAGT
GAGGCTCTGCTCCTAAGGGAGGGTGGCTCATAGGGGGTCCAGGGCCCAGGTGTGACCCTGGCCAGGCCTG
TGTGCATGTGTGTGCACACGCATGTGTGTGCACACGCATGTGTGTGCGATTGCTCCCTGAATGCAGAAAG
GCAGGGAGCAGTGGTGAGTTGCCTTTGAACACCAACATGGTTTCCACTGCTCCCCACCCTCCTGCAACCG
ACAGGGGTTTATAGGGCCAGGTTGGTCCTGCTCGGAGTGGAGGCCAGCTTGTGGTGTGAGCAGATGCTCA
CAAGGTTGGCCCTGCTCCAGCCTCTCTGCACCCTCCCTCTCCCTGGCTGTCAGAAAGCTCCCTAAGGAGC
TACTTCTATATGTGTGTCACCATGGGAGAAGACGCTGGTCCCTAGGCCAACCCCGGCCCAGCAGCTGTGG
GTGCAGGACTCCCTGGGAAGGTAGGCAAGGGGGTCTGGAAGCGCAGTAGGCTGGTAACCCTTCTCCAGTA
CTGTGGCTCTTCAGGCCAGGGCAGGTGTTCTTAACAGGACTGCACAGCCTCAAGGGCGCACAGGGCCAGA
GGTGGGGGTGGGGGGCCGAGGCAGAGGCCAGACCTGCAAGGCTCTTGTAATGGAACACACCTGATAAGCC
CTCAGGGCTGTGGTTACCCTGAAGTGGGCGAATGCCCTAGGTGCTGTCCACAGCCTTAAAAATCACACAA
GGAAGCATGCATGTCCCCGCGCTGGTGCATGCCCCCGCCACCCAGCCTGCTGCCCTGGCTCTGCCCACCC
CTTCTGTCCACCTCTCTTCTGACCACAGGCCCCCTCCACACCTGAGTGCAGGTGGGAGCTGGGCCCGCAA
GGGCCTGGGTTCACATGGCCTGAGGCACATCAGAAGGGATACCAGGCTGTGTGAAACTGAGGCTTCTGAG
GACAGCTACCTCCCCTCCCCTTGGGTGTGCAGGCTGCCGAGTGGTGGGTATTGCCTTATAGCTGCTCTGC
CCTGCAAGGCCTATCATGGCAGCTGAGGGGGGTCAGGCTGCCCAGAAGTGGCCTTGTCTCTCCTGGATTT
TTGAGTGCACCTGGCTCCCCTGACTAGTCCCGGGGCCACCTTTCCCCTCCTCTCATCTGCAGAGCCTGAG
GGTTCTCTTCAGAAACCCCTCTGGCCTTCCCCAAACTCTCCTGAGCTCCCATGGTGGGGACACCACTCAG
TAGGGCCCTTCCTTCCCTAGAGGCTGCTGTGTCCTCCTCTGCCCACCCTCACTGTTCCCAAACTTGCCAA
GTCGCGCGCGCATGTGTGTGTGTGTGTGTGTATGCACACACACGTGTGACCCAGTTCTCACTCTCCACTC
TGAATCCCCAGAGTCAGGTCTCTCAGGTGGGCCCCTAAAGCCACTATTCCATGTGGCAAATCATGACACT
GTGATCTTCCAGATGGGGTGATTCCTCTCCCACAAAGGGTGGCTGAACAGTGCAAGTGGCTCCCCAGGGC
CGCAGGGCAGGGCCCGGTTCCCTCTGAAAGGCCCTACCCAGCTGATCTGTGTTGACTGAGCTCACACCCC
TCCCTGGGTATCTGAGGGCACATGCTCACCCTCCCTCCTGCTCCTGGCTCACACCTCCCCTTCCCATCTC
CCACTGGTGGCTGGCCCTGGCTTTCAGTTTATCCTCACTTTCTGCCTCCAGATCTCAACTCCTTCCAACA
GACCTCTAAGTGGATCGACGACGTCAGGACAGAGAGGGGCAGTGATGTTATCATCATGCTGGTGGGCAAC
AAGACGGACCTGGCTGATAAGAGGTAGGTGCAAGGGCACACAGGTGCAGGGTGCCACCCTCCACTCCTCT
GTAACTGCTCCTGGACAGGGAGCTCTGGGATGCATGGGTGGTGTGTCTGTGCTTCTGACCATGCATCCGG
AAAAGGTCCCTTGAGACACTAGGAGCTTTGAGGACACCTGGTAGACTCTCATCCATGGGTATCTCATCTG
TGAAGCCCTGTCTTCCACCACCAGGCAGGGGCGGGGCACTTCTCCGTTTGCTGTCTACACAGAGCACTGT
GCACCTGGCTTGACCCAGCAGTGCATGGTCTAAGGCCCTGCCATGGCCACAGCCCTGCCCCAGACACAGC
AGTCCTCTAGGCCCCAGCGTGTGCACTGTGAACTGTCTGTGCCTGTGAAGTGTCTGCACCAATGTGCGTG
GGGCTGTGAATGCCCTGGGCCCTAGGTGTGAGCATGCAGAGCATTCAGACCACAAACCTATGGCCGTAGC
CTGCTTAGCCTGGTTTGTGGCTGCGTCTGGCATGAAGCTTTCTCTGAAGAGGCTCCTCCATGCTGCCCTC
CATGCACAGGGAGAACTGAGGTGCCATGTGTGTACATTGTCCACTCATTTTACATGACCCTGACTTGGGC
AGATAAAGTGACAGGCCATGGACTCCAGCAAGCAGTGTGGGCTTCCTGCAGCGTCCCTTGACCACCTGAG
GAATCATCTGAATTAGAGCCTGCTTTACTAGAGTTCTGTCCTTAAAGTAAGGAAAACTGGGCTCAAATCC
CAGTACAAAGGGTACCCAGGCCCTTCATACCCTCACCCACTTCTGCCTGCCCCATCATGGAGCCCTCTCT
GCTAAGTAGCTTTGTGGCCCTGGGGGAGACACTTTTAGCATTAGTGTTCTCACAGGGAAAATGCGACTGA
TAATACCACCCAGAGTGGCACTGCCTGGCTTGAATCTCTTCATAAGCACAGGCTCTGCAGTAGTGACTAC
TGGGAAAGGCAGGTATGGGGCAGCCCTACCCCAGGGCCTGCTTTGAAGCTGAGAAGACACAAAGGAAGGA
GTGGGGGAAGCGGATAGTGTCCTACTGTTCCCTGGTGCCTGCTTTGTGCTAGCGAGACTGTGGTTGGTGC
CCGGGAGGGACCCTTGGGATCAGAGCCTGTCTTCAGGACTGACCAAGGGCCCAGTGGGCTCCCTCTGCCC
TGCTGGGTGGGTCCCTCCTCATATCTGCCAGGGTCTGCTTCTCTGCTGAGCTGATTTCCCCCCTTCCCCT
CATCTCTAGGCAGATAACCATCGAGGAGGGGGAGCAGCGCGCCAAAGAACTGAGCGTCATGTTCATTGAG
ACCAGTGCGAAGACTGGCTACAACGTGAAGCAGGTGAGGACACACCTGGCACGGGGCCCGGCACGGGGCC
CGGCACGGTGTAGCCCTGTGGTCAGCTGCAGGCTCAGAGCTGCCTTGCCCTGAAGGGCTGGTGATGGGAT
TGGGGCAGCCAACCAGAACACCCATAGATGCGCCCAGGGAGGGGAGAGCTAATTTCCGGAGCTGGTAAAG
CCCAAAAGAGGGAGGGACCACTCCGGGCATGGCATCTGGAGAACATAAGTCACCAGCCACCGTAGTAGTT
GCCAGTCACGGCCCACGTAGCAGGCCCTAAGAGTGTCCAGCCACCAGCTTCCAGGAGGCTGAGGGAAGCA
GTGGTTCCCTAGCCCCTCACCCCACTTTGGATGACAGGTGCAGGAAAATTCAATCTGATCAGCATTTGGG
GTTCATGCCTTGCGCTTAGCAAGTGCTCAGGGCTGTGGAGGGCAACTCAGGCCTGGGGGAAAGAGCCTAC
CATGATGTGGTTGGAACTCACAGGAGGGCGAGGCAGTGGAGTTAGGGCTGAGCCTTGCAGCATGAGGGCC
CCTGAGTAGTCCATGGGTGGAATTTAGCAGGTCAGCAGACAGGGCTGGAAAACATTGCACCTTTATCTTC
ACTAACCACTGACCCGTCCGTAGCACTGAAGGACGTCTGATTTGTCCTTCAATGATGAATGTAGGCAATG
ACACTCAGTCACATTAGCAGTGCCTATGGCTTTACCACCAATAGAAATCACAGATGCCTTCCCATCACCT
CATAGTGTTGCAGGTATCTTGAACTTCACATCTATCAGTGTTTCAGCATCACACTACTTATTAGATCTCT
TGCTAGTTCTCGCTGTTCAATGTGTTTCATAAGGAAGCACAAATGTCGATGAAGTTTTATTTCAGTACTC
AGTAGTCCCCTTTATCATCCTATGTAATTGATTTCACATACTTCAAAGGAGTCCATAGGATTAACCAGAT
TGCCAGAGGGGTCCTTGGCTCACGGAAGGGTTAAGAACTCCTGCCGTTGGGGATGAGAAGGACTTGGAGA
GGTGGTGAGGAGGGAGGGCATGCCAAGCAGGGGAGCAGCAAAGGTGTGGCTGCAGGGTGCCCGAGGTATG
GGGAGGGGACTCTGGGTGAGGAGTGTGGTTGGCCTGGCGGTCGCTTCTGCCCTGCCACCCCAGCATTGCT
GGGAACCCTTGGCAAGCTGTGTATACAGCACTGGCCTTCTGGATGCAGTGATGGTTTGCCCTGTGTGGAC
AGTGCTGGTGGCAGTGGGGAAGGTGGGCTGGCAGAACGACAAGAAGAGACAAGAAGGGTGGAAGGGACAC
TATGTGACACTGGGGGCCCCAAAGCTCCAGGACTCAGTGCCCCACCCTAACTAGAGTGTCCTCATCAGGG
CTTCACTCACTCCCATCTGTTCCCAGCCTCCCTTGGTGTGGGGCTGAAGCTGTCATGGTCCTTCCTGCTC
CCTGGGGGCATGCAGGAGAGACTGGGCTGCTACTCTTTGGGAGCCCTAAAGTCATGGGAAGCAGGGAAGC
AGAGGGAGGGCAGGGCAGCACCAGGAGGTCCATAGGGCAGGTAGCATCTGCCTCAGCCGTCAGGGTCAGG
GAGGCACTGATGCCCCTACCCCCAGAGTGCTCTGGAACCCATGCAGTGGTTTAGCAGGAGTTAAATGTGG
TCAGTTTCGAACCCTGTGTGGCTCAGAGGAGTGTCCATGCAGTGCAGACTCCTGGATCTAAATGTGAGCA
TATCCACTCACAGAAACCCATACCTGACCCTAGAAACCCATACCTGATCCTAGAGCACCCTTCAAAATCC
CTAGTGGCATTTTGGCCACTAGTAGAGGCTGTGACTGTTTTGAGAGCCAGACAGGCAGATCAGAGCCGCC
CAGGGCCAATGCCATGAAGGAAAGCTCAGGAAGGATGGCCTGGGCCCTTTTACCCTTGCCTACTAATGGC
TACCCCACTGCAGAGCCTCCTCTGCAGCCCGCCCACAAGGGTCTCACACCATGCTGGGCCCACCAGCAGC
TTCCCCCCTTGGGCCCTCATGGCCTTTAGCCCTGTCACCCAGCCCGGGTGCTCAGCTGGGGCTGGGCTCT
GGGGTGAGGCCATGAATAAGACTCACCCTGTCTTCAGAGAGCTCATGCAGTGGAGGGTATGAGCCCAGAG
CCCACCCAGTTCCTGCAATTCACATTTGTGAGGACAGCGGGAGTGCTCCTGGGTTAGTCCTTTCAGGCTA
CCGTAACAAATCACTATAGGCAGGGTAGCATATAAACAGCAGAAATGTATTGCTTACAGCTCTGGAGGCT
GGGAACTCCATGGTCAAGGTGCAGGCATATTCAGTGTCTGGTGAGGGCCCATTTCCTCCTAGATGGCACC
TTCCTGCTGCATCCTCACAAGGTGGAAGGGGCAGACAGGCTCCCTCGGGCCTCATTCATAAGGGCACTCA
TCCCGTTCATGAGGACTCTACCCTCCTGACTAAGCACCTCCTAAAGCCCCACCTCTTTATACTGTCACAT
TGGAGATTCTGTTTCAACATAGGAATTTGCAGGGGGACCGGCATTCAGAACCTAGCAGTTCAGAGTCTTC
TGTGCCTCATACTTCTGCCTCGTACCTGTCACCTCCAAGATTCCATCTCTGCCAGCACCCCATCTGCCAC
TTGCCACCTGTCCCCTGCCACCCACACCTGCCCAAACACACACACACACACACACACACACACACAGAAA
ACCCACATGTACGCAGACACACACCTTACCAAGGACACACACTATGCACACATTCCCCCCACACTTACAC
AAACACACACATAAAGATGCCACACGTTCACACACACCACACTCAAACATTCCCCCATATGCTTACACAA
ACATACATGCACCTACCCACACGCACCACTCACAACACACACATTTTCCACGCAGTCCTACAGATAAAAC
ACATACACTGTACACACACTCCTACACACAATAAACAACTCACACTCCTACACATCCAAACAAGCCCACA
AAATTGTGTTCACACTCCTGCATCCCTCGCGCTCTCAGAGAAGCCTCACCACTGTGCCCATGCAGCCCTG
GCCCTGCTGTTCGCTGCCTTGGCGCCGGACCTGCAGGGCTCCCCAAGCAGAGTCATTCATCTCAAGGGCA
AGTCACTTCCCCATGCCTGGTCCCCATGAGGGCTGTTTGCAGAAGCCCACCTTAAGGCCTGTCACCCAGA
GGCCCAAACCATTTTCTGCACCAGAGGAAGTCCCTTGCAGTCACTGCCATGACCTAAGCTCATCCCAGTG
ACCCTATGGCCAGTCTGCTTGGGTGAGAGGCCACAGCTTTGTTCCTTCCTTCCACAAGCTACTGAGGCCT
GCCCAGACCCAATGCTGTGAGAGGTGGGGGAGAGAGAACATAGGCACAGCCCCTGCCTTTGAGGGCACCG
TCCGCTGGGCAGATGGGAGAGTAAAGCTGCAGGTTGGGAGGGGAAGAGGGGTGAGGGTGCAGGTGCAGTG
AGGAGCAGAGGGGAGGGCCAGGGTTGGGGTTCACACTGCATTTCTTCATCTTTCCAGCTTTTTCGACGTG
TGGCGTCGGCTCTACCCGGAATGGAGAATGTCCAGGAGAAAAGCAAAGAAGGGAGTATCCTTTTCCTTTG
ACAAGTGAGTGAAAAGATGTATAGAAGCCATTTATTCACTGAATATGTTAGTGCAGTGGACAGACACTCG
CCCAGTCCCCGCCCAGCCTTCCCTGGTCTGCCGCGAATGGCACGGGGCCTCCTCTAGGCGGGATGCAGCT
GAACCCTATGGTTTGGAGCCCCCGTCTGGCTCTAACCATCTACTTCTTCTTCCAGAATAGCAGGAAGATA
CCAAAGGGGTGTGCACAGAGTGATGCTGTGGGGTGGGGAGTGAGAAACAGCGACTTTGCCCTTCTTTACA
GCGAGCATGTTTATGGGTTTCATGAACACAGGCAGAGTCTCCACTGCAGGGAAGTCTCTGTCACTGGGGA
TATGATCTCAACGTGGTTCAGTCTACTCAAGACAGCTCCCCCCTTCAGACAGATGCCCCCTCCTTGTCCC
GCCCACCTTCTCTACCCAGCCCTGAACAGCTCCTGAGGCTCCCCACTGATCTGTGGGACCATTGCGCATC
ACCCACAAACCAGCCCCAACCTGAAGAGACACCTAGATGCTGAAACTGAGGTTCAGAGAGGTAGTCTGGT
TTGCCCAGTGTCACACAGCATGTAAGTTACAGTTAGGATTCACACTCAGGCTTGCTTCCTCCATATCCAG
CCCTATTCCATGTGGGACACTGTCCCTCTAAGGGCATTTGAGAGAATCAGATGAAAACTGTTTCAGCAGG
GGAAGCCATTGCCCACACCTGGGCCTCCCCTGCATCTAGTCAGATGTCCCTGCCTGAGTAAGCCACCAGA
AAACAAGCATGCAGATCCCTGGCTGGCATCCTTTACAGTGGACCTGTCCCTACACAGCAGCTCTCCAACA
TCCCCCACTGATCCTATAGGCCTCTGAGGGTAGACATCCCTGACAGATCAAAGAAGGGCCCTGGGAAGAT
GGAAGGGCACCTTTGGGCTGATGAGAATCAAAAGCAAAAGAAGCACACAGCTTAATGTTGGAGGCCTCAT
GGAGCTGCTGGGGCAGGGCGGGCAGGACACCAGGAGTCCCAGGGCTGCAGCAAGGGGGAGCCTGACACAG
GCCTGGAGGGAAGCTAGGCCTCTCCAGCCTTGCTCAAGGAGCTCTCAGAGCAGACTCGAGACCCACCCCC
CGGGGGGGTCAGGAAGGCTCACCAAGGAGCAGGTGCCCCAGCAGTCCCACGGTCTGTGTGTCACCAGAGG
ACAAGCAGTGGCCGAGAGGAGGGGGTTTGAGGAGTATCTCCTGGTTGGTCAGTTATGAGACTCCAATCAC
AGATAAGGAAACTGAGGCTCAGAAACTTAAACTAGGGCACCCCACGGCTCTCAGTAGCAGAGACAGGCTC
CAGAGCTCAGGAGAGAGCCCCTGTGGAATCACCTGTCACATGGGACTCCTTTGAGGTTCCAGGAACATAC
AGAAGCCAACCTGAAGGGCAAACATAGGAGCAGGAACAAGGGAGAGGGGCAGACAAGGTGCAGGCGATCT
CCGCTGTGCCTGCCAGCAAGGGGAAGGGTTGCACTGCCCGCTCTCCATCCCACCAGCTCCCAAGGTGTGG
CACACTGGCCGGGGTGAGTGGTGATCGTTTCATTCACATTCTCCCAGAGTCGCGCTGTTGAGGGCTCCAG
GCTGGGCCCACAGTCAGGTCAGACTGGGCTGAGTGCAGGGTGACAGTGAGCTCGTCATCGTCCTGGTATG
CCTAGCGTTTCATCCAGCTACCACTGACCTTTGGGCCCCTTGGGAGAAAGGATACATGGCAGGTTGCAAA
CAGACTGCACAGGGTGTTGTGACCTGTGTCCCTTTTTCTAGCTAAGGGGTCCCCTCATCTGGGTAGATGT
ACCCCCATCCTTTCACTTCTGCCCAGGAGCCAGCTTGCTGGGCCTCTCACGAGGCAGGTGCTAGAGCGGC
CACCAGAGGTCCCAGAGGGACCAGGCAGAGGCAGCCGTCATTCTGCTTCTTTCGGGATGGGGTGTTGGCA
TTTCTGCTTACTCACAGTCCAAATCCAGGTGTTTTGAAGGTGTGTGCTTTGGGGTCATGTTCATAATTGT
GTTCCACATTGAGAGCAGCCACACAGAAGCACTGCTTTGTGGCGCTGTATAAACTGTTCACACCTGGCCT
CACTTGCTCAGCTCAGGACTTGGTGAATGTGTGGCATGAGGTTTCCGTCTGCTGCTTGCAGGTCTAGATG
GGGGGCTGCTGCACCTCTTCTTCACGTTCGAGACAGGAGGACCCCTCAGGGCCGCTCTGGCCAATACTTC
TGCCCAGCAGCACCCTCCACTGGTCTGCTCTCAACAAAGTAGGAGCCAAGGGGCCACCAAATGGTAGAGC
CATAAGCCCCTTACTGGTTCTACATACACAACCCCCCAACCCCCACTTTCTACCCAAATACAGTATGCGC
CCATCACATGGGCCCCAGACTGTTTCTGAACAAGGCTGGCTTACGCAAAGAAAGAGCAGTAGTGTGGGCA
CTTCATGAAGTGGCTCTAAGCAGGTGAGGACAGTGACGCCGCAAACCAGATAGAGCATGCTCAAGGCCTC
CCAGCTCCTTAATTCAGAGCTGGAACCACATCCAAATGACTGGATTCCACAGCCCTGTTGCACTCTCTGA
TGGGGCTGTGGTGTGCTTTGACATTCCAGATACAGACAGGATTGTGCTGCAGTAGAGTCAGAGGCACTGG
GATTGGGGAAGCATATCCAGCAGTAGGGGGTAAGTTTGTAACAGGCTGTCTGGGCCCACCCATCACTTCC
AGGAACCAGGGAGAAGGGACATTGGGCACTGAATGTGGCCCGTGATGGGCCACACTTGACCCAGAGGGAA
GAGGCGGGTGTGCTCTGGCCATGCCTGCCGGAAAGGCACATATCTAACCTCTTGAGGCTTGTGAGAAACC
GTGAGTTTCCCCTTCTGGGACATTATTTAGACATGGGTCCAGTCATTCTTCCCTGTCCTCTCAATCAGCG
GGCAGCCCCTGAGCACTGCATTTCAGCAGCATGTGGCGTAACAAGGAAGGAGGTGTCATTAAATGAGAGC
CTTTGGGAAGGCTCCTAGCGGAAGAGCTGGGCCTGTGCTCTCCAATTCTGCAGCCACATGTGGGTGTTCC
GACTTAAATTAGTTAAAATTCAGTTGAATTAAAAGTTCATTTCCTCACTTGCACTAGCATCCTTGCAAGT
GCCCAGTTGCCCATGTTGCTAATGGTGACATCTTGGACAGTGCAGATATAGAATATTTCCATCACTGGAG
AAAGTTCTTTGGACAGTGCTGTTCTAGAAGTATCAGTAGAATTGGTTTCCCGGATGTCTGTGCATTGGGA
CTCTCCAAAGAACTTGTTAAAAACACACATCTGGGGCCCATCCCCACATCTACCAAGGTAGGACCCAGTG
AGACGTATTTTTAACAAACATCCCAGAAAATTTTGATGCAATCCATTTGAGAACTATTGCTGTGCAGTGT
ACAAAAGTATTGTGATCAAATTGCGCATGAGATTGTTTTAGTGGTCGTAGGTGACTTCAATACTGGGCAG
AACGTAATGCTGTAACAGTGGCCAACCTTCAAGCTAGACCACTAGTTCTCAACCAGGACTGATAGCCCTT
CCCTCCCCTCCCCACAGGGGACATTTGGCAATGTCTTCAGACGTTTTGGCTCACCATAAATTGGGGGAGG
GGGAATGGGGTTTAACTGGCATCTCATAGATAGTGGTCAGGGATGCTTCTGAACATCCTCCGTGCACAGG
ACGCCCTCATAACAAGGAATTGTCTGGCCCAAAATGTCAGTAGTGTTGAGTTTGAGAAGCCCATCCACAA
GGGGCTTAGCACAGCCAGGTCAGTGAAAGTAGAAGGCTGGGGTCAAGATAGCCTTGGGTTTGTACCTGGG
CACCTCAGCTTCCCAGCTGGAAGGTGGCTCTTTTACTCCCCACTGCACGAGGTCTAATGTGGAAGTGAGG
TAGCTCTCAGCAGGAGAGGCCACTAATGCGTGCACCCTGGCCCAAGGAATGAGGACATTTTTACAGGTGG
AGATGGGGAGGAAGGCTGGGTGTGGGGGAGGGAGCATCACTTCTAGAAGGAAAGAACAGCATGAACAAAG
GAAAGAAGGAAAGCATAGGACGTGTTTAAAGCAGGGAAAATCTGGTTGGTGGAGCCTTGGTGTGTGATTG
AGGGAAGGGGATAAGCTAGCGTCAACAGGTGCTTGGGGTGACCCTAAATGGGGAGGCCTTAAATGCCAGA
GTGAGAAATCCGTGGACACTTTGTTTCTGTAGCAGAGTGTGACATGATTGTCACCAGAGCTGGCTTTGGG
AAGCCACCATGCTGAGAGAGAGCTGGCTGGGCTCAGAGGGGCACTCAGAGAAGACCAGGAGACCTCAAGG
AGGCCTGTGCCCAGGCCAGCAATACAGAACAGGACTGCACTGCCGGCATGGGCCATAGAACTGCAGCTGG
CAGAGAGAAACAGCAGGGCATTCCTGGCAGTGGGTTTACCAAAAATGATGGGCAGTAGAGAAGCAGGCTG
CTTGACTAGGGTAGAGAGAACAAAAGGTGGGCTGCAGTGGGCAGTGGGAAGGAAGGCACTGGCTTAGTAT
CTATTTACTCATTTATAGGCTGCCATGGTATAAGATACATATACATACACACAAGATAAAGTTGTATGTG
TATGTGTGTACACACACACATGGGGAAAATAAAGGTAGGAAAAGCAAAGAAAAGTCAGAGATAAAGTCAT
ACTCAAAATGTATGCCATGGCCGCATGGTTGTTAGAGGTGGCCAAAAAGTTGGCTCTGAGCATCCTCACA
GCCAAGGCAAGGAAAGAGAATGTGAACTTCAGGAGAAGCATGGCTTTTCATAGTACTTAAAACAAAAGAA
CCTCTCCTGTTGTCCTCAAATAGGAGCCACAAAAGCATCCTTAGGATGCCAGCAAGAAGTTCCAAAACAG
TGTTTCTTATACCACCACACCCCACACACCCCTGTCTTAAGGCAATCCCTGTCCAGGAAGAGCAATTCTG
TGAGGTATCAAAGGGCCTGATCCTCAGCTATAATTAGGTCTGGTTTGATGCAAGGCTAAAATCTCACAGT
GTTGGTTTTGAGCCCGATGAGGAAGGCAGAGGGCCAGCCTGGCTGTGCATTTGGAACCAGTCCCTGTGGT
GGTTCTGATAAGTGCTTCTTTAGCCCACAGCCCCCGGGGGCACCAGTGGAAAGGGCTGGCGAGGGGGCTG
TGCTTAGGCCCCATGGATGTGGCTTCTTACAGTAACCATCCTCAGGATCTAAGTGATGTGTTCTGACTAA
AGCTTGAAGGGTTTGCTCCTGCTGCCAATTAGGCAAGGCCCACATACCCCTGTGCTTTGAGATGTGAACC
CAGCAGGCTCTTCATCTCCATTGCACTTTGAGTGGGGCTGACAGGTAACTGGGCTGAGGCCAGGGCTGGG
AGCTGGCATTTGAGCACTGGTCGAGGGAAGGCTTCTGGGAAAGGGACTCCCTGGCCTTGGTATAGATGGT
GATCCCACGGCCCAGGCAAGACAGGCTTTGCTTGGGTGGACAGGCGCTGGGGGAAGTGGACGGGTGAAGG
GAAGAGGAGGCAAGGTGGAGTTGGTATTGCATCCTTCCTAGCCCAGCCAAACCAAGAGCCAAGTCCCTAG
GTGTTTGCAGAGAAACAGAGCAGTAGCCAAAGGGTACAGTGCACAGGGCTAAGTGGGGTGGCAGCTTGTC
CAGGAGAGTCATCTTCCTTAGCCCGTCCCCCTGCAGTGATCGACATCAAGCTGGACAAACCCCAGGAGCC
CCCGGCCAGCGAGGGCGGCTGCTCCTGCTAATGCAGAGCCGACCTGTGGCTTCCCATGACACTCCTTGCT
TGTTGTGTTGCTTCCTATTGGCTAGCTTCCTAAGGGGGGAGGGAACCGAGTTATCAAGATGGGAGGATTT
TTCTTTTCTCTCTGTCTTTAGGAGTAGGGTGGGATGGGGAGGGAGGCTGGGCATCAGGGATCACATCACT
CTTAACGGCTGTTACTTAAACAACTATTTTTTGGTTTGGTTGTAATATATTGTACTTTATTAAGATTGCC
AAAAACTGTTAAAATTTAAAAAAAATTTAAATCATGTGTATACAATTTTTTGCCAGATAAAAATGTAGTC
ATTTTTATTTGAAAGATGTGCTTTTTGTTTTTGTATATTTGTAAACTTATAGAGAACCTTTTCCACACAC
CTCCTCCTTCCTGTTCTCTTTGAACCATTCATCACCTCTGCCTTCCTCCTATCCCCAGCCCAATAAATTA
AAACAATTAACTGAGCAAATTAATTAGGCTTCAGTCTGGGGCCATCTGGCCCCACTCTCTAGGCCCTACT
CCAGTTAAATCAAACATTGGTTGAACACATCAGCCTCTGAAAAGGTAGCTCTGACTCTTGTCTTTCCATG
GCCAGAGTGGGTCGTCAACCTCGTGTGGAGCTGAGACCAAGGTCTGTGGGCTCTAGGCAGCCCGGCAGCA
GCCCCCAGAGGAACTGCTCCCACCTCTCCTTCTCTTTCCCTGAAAAGCCTTGAAGGGGGTTGAGGGTACA
GGCAACCTTGTGTTCTCTGTGCCCTCAGGGACTGGCAGAGCAAGAGGCCATCAGAAGGACGAGGTGCTGT
TGGCAATTGCAGCTGCGTGGTGCGTGGTTTATAGACTGGGGTGCCATGTGTACTTCTGAAAATATTTTAA
CCAGCCCACCCAGCAGTCAGTACACATGCCTGGGCCAAGTCTAAGAAACTCAGTGTGTGATGTTGGGCAG
TGTGCCATCCTGAAGCCTTCCTCCTGGATGTCAGGGAAGCTGGACCACCTGGAGTTGATCCTCTGTGATG
GGGAGGCGGGGGGGGGGGGGGGTGTTGTCTGCAGAACCACCACCTTGAAAGATCCATGTTCTTAGAGCCT
CAGCTGCTAATATTGTTGAGTGCTTTCTGGAAAAGCTGATTTGTTGCTATGTTTTCACCCCAGTGGCATG
GCGCAGTTGAAGTCTTTGTGGACATATCTTTGTGGCTACTGTGGCTGCGCTGTGGTCACCTGAGGATGTG
GTTCAGGTGGGGTCTCCAGAAGGCTGCTTGCCCCCAGAGAGCCATCTCTTCCATTTTTGATTCATTCCTG
AGAATGGCTTAGTCCTGAGAATCTGCAAAGTCTCTGAAGAGAACTCTCTCTTCCCACTAAGTGATATAAC
CTACTTGGCCAGCATTCCAAATAGACTCAGACTCACTGCCCTGAGCCTGCACAGAGCCAGGTGCAATGGG
CAGTGAGTCTGGCCAAAATGTAAACCCCACCCTCCCTGCTGTCATTGGCTCTCCCTCCAGGCTGCTGCTG
GGCATGTCATAGATGGGATGAATGACCATCTGGGGTATGGTGGGCTGAACTCCACAAGGTCGTTGGAATG
AATTTTGAAACTTCTCAGAAATTAATCTGAAATGGCAGGCAGTAAGTGCAGCAGAATCTGGCAGGACTGA
TATTTGTGCAGTGGGCCTGCCACACACCCTCTGCCTAGGAGGCTGTATGCCAGAGCTCATCTCTAGCAAG
GAATTTATCTTCAGGTGAAGAAGAGAGGAAGCAGGATCCCACTGAGCACCTCTTGCAGAAGGCAAGGACC
AATGTTTGAACGTGTTTCAGATGTCATGTGTTTAGTTTTTATATGTGTGTACATAATTATATATAGAGAT
AGACTTGTGTATACACCTTCCACAAACACATCCTCACGGAGATACATGTGCTCAATGTATATTCTTGTTT
CTCCTGCTCTCAAGTTGGAACTTGCATTGTGATAAGTTTTGGAAATTCAAAAGTACTGCAACGTGTTGAA
ATAAGAAATACTTAATATTGTCAAATAACTTGTGATTGTCTTGCCATCAATAAAATAATGCATAGTTGTG
TTAAACTACAGGCAAACATAGCCCACGGGAATATCTCCACCATTTCTCTATGTACTTTATATGCACGGGT
GTGCATGCCTGTGTGGGCCAGTGTCCCCCCACCATGGGCACACCCCCAAGTGGTGCTTGTTGGAAGAGTG
CTTACTTACCTCGAGAGAATTTACAGGAGTCCACATGACAGTGTCCTGCCTGGTTGGCAGCGGGGGCCAT
CACCTGTGGCCTCTGAGACCGCCCCATTTACAGGAATCTGCACTGGGTCTCAGTGAATGAACGTATGGTA
GATTTTTATTTTCTTGGCACTAAAGATTAGCTTTTTTAAAAAAACATCCCATAAAACAGTAGAAACTCAG
TTCCCTTCTCAGGGGCACACACATCCAAGGCACCGTGGACTGTGTTTCCATCCTGGCCTAGAGGAGCAGT
AAAAGATAGCATTTGGTTTGACATGGCCTTAGTGAGTCCTGCCTTTCCTAGCATGCACACAACATCCTCT
CTTTGAGTTTGGGTTCTCCCCAGCCCCTTGGCCTCTGAGGCTGAGGGTGGCTTCCTCCCATCCTCCCATC
TCAGTGGTTCCATGGCGCTCCCTTCGGCACGTGCGGTTTCCGCCCCTCCCTTCCTGGGGTGTGGAAAGGC
AGCCAGACCTCTTTTTCAGAAGTTGTTGGTTCCCTGATTTTTGTCTTGAGCAGGCACCATCCCTGTCCCC
TTGCTTGCAGGTAGAATTGGCACAGAGTTAAGGTTCCATAATAAACTGTTAGGTGCTCTTGAAGAAGATG
TCATGGCATTGCCTCAGCACCCCAGCAAGAGCATTGCATCTTTGCTGAATCCAGATGGGTTAGCAGAGGG
CAAATTGGAGGATTCCTTTGAGGAAATGTTCTCAATTGAGCAATTCAGTCCCTCTCCCCAAAGACTAGAC
GTGACCTGGCAGATTAGCAGGATCCTCTTGGCCTTCTTTTTTGGGGATGGTGGGAGTGAAGGGGGTGAAT
CTTAAAAGCTTATCAACAAAGCTCCTGTTCTGCAGGCATCAGGAAAGTTGAGTGTAAGATGGAAAAAGCA
AACAGCCTATTTACAAGTGGAAACACATTGGTTCTGATCACTCCTAACTTGCTGGGTGATCATACATACA
TCAACATGAAGGTGTGACATCAGAATCATGAGCACCATGGCTGCAGTTGTGTGATCCAAGGCTAGTTGGC
TTGGTAGCTGTTGGCAGCAGTTTCCCTTGGTCAGAGGCCTCCTTGCTATCTATCGCTGCTCTCTTCTTGT
CATCCTTAAACATTCAGTCATGTTCCCAGGTCTGAGGGGAGCAATGGCCAGACCAGTTTCAGTATCTCTG
GCAACAGAAACAAGGATTGAAATGGTGGTAGTTAAGAGTGCAGACACATCTGGGGTGCAACGTCTGCATC
ATCCTGGTGGCTAGCCAGTGCGGGGAGGCCCTGCCCTGGGATACCAGTTGTGACAATAAAGGGATGGCCC
TCCTGGCTTCAGCATCCTGCAGGTAGAGCAGCCTCATTCCTTCACAAGATGGAAGGCAGGTGAGGATGTC
AGGATGAGATGGGACCAGGTAGCCGAGGACAGTGAGGCATGTCTGAGCCACTTGAGGGGCAGCTGAACAG
AAGGATGTCTGTGTGCATCAACCGAGCAGATTGTGCGCACACTCGTTTCCGAACTGCCTCTCCCCAAAGA
ATGTGAGGCATTGTCACCTTGTTACAGCTATGTGAAGCAGAGCTGTCACAACTTTTCCTGTCTACAGGCA
GGAAAACCAGGATGGAGAAGGGCAACTTGCTTATTAAGATCACCCAGCAAGTTAGGAGTGATCAGAACCA
GTGAAGTTTCCATTTTTCATCCATTGCTGCCCTGTGTGTGAACAAATGTGACTCCATATTTATTACACAG
CAAGGACTGCTGTGTGTGGGTGTGTTCCTCAGACCATCTCTACTCTTTTTGGTTAGGACACTAGGTTCTT
CCCAGCATATGAGTGAGAATATTGGTGGTATTTTGGGCTCCTCTACTGCACAAGGGCCAGTCCCCAAGTC
TACTCTGATTGATTGTCATGGGCACCCACACAGATCAGGACACCCTTTGAATTTTTGTTTGACATGAACT
ATTTAGGTGTTCCAAAACCTTGGGATCTCAATGTGCAAACTCCTTGGACTCCAGACCCCTGTCTGGTTGG
CACTCCCAGCAGTGTGTGGTTTCATCAGGAAATCCTTGCAGCAGGGGTTTAGATGGTTCCTTCAGGAGGT
GGGAAGCCTCCATAGCATTGAAGGCCAGCCTGCATCTCGCGGTGTGTGTCCACTTCTCCCCTCACTTTCC
CCTCACCCATCCCTTTAAAGTGATTGGCTGTGAGGAGCACAGAGGGTGGCAATTCCATCTGTTATGGGCT
CAGAAGACATAGCCGCCATGTTGATTTCACATTAAACTGTGTAAAATGTGGAAAAGACTCAGCATGTTGG
TCAAACAATAAACTGTCCTAATTCTCAAAACAAAGCTTCTTGTCTGTGTTTAAGTGGTGCAGTCATAAAT
GGGGGGGTTCAAAAACAGTTCCTGAAGTGTGTGCTCCACAGAGTCCCTTTTGTCATGCCTGGGTGGTATG
TCCACTACCTAAAGCCCTTTGCTGCTTTGCTTCTTCCTCCTACTGGTGGAGTTGGGCTGTCCACCAACTC
CGAAGGGGTAAAGATGGGACACACTCATGAGAGTAAATCCACACGCATTCCCCTAATCTCCATTCTGCTG
AAAGGCTGATGGGAAGGGAGCCTCCAGGCAGAACTGAGTCAGGGGTACGATATATTCCTTCATCCCCACT
CTGCTTGCCATGGCTAGGGGATTAGGAAGGGGGGAGAGGGTACAGGGACTGGATCTCTCATGCTTCTGGT
GATTTTCAGAAAGCCATTTAAAGGCAAGAACTTAAGGTGAACTATAATGACCTTTGACAACATCTAGCTG
CCATATTCCATCCCCAGCCCCACACCACACCAGTTAGGAACATCCCTGAGTGGCCCCTCCAGGTCTCAGT
TATCTCCAGACTAAAAGATGCTCAGAAAGCCAGCACAGTCCTGGGACCAATCCAAAGAGGAACAAAGGCC
AAGATTAGGTAAATTGCCCTTGCCCGGGAATCTCACTCTTCTTCAAGTCCAACTTCACTAATTACCTTTC
CTTTTAAAGCTTATAAAGGTTCCAGAACACCCTCCAATCTGCTGCCACCTCAAGGAAGCCATCTTTGATT
AGAAGAGACATTGAGGCCAGGAGTTTGAGAACAGACTGGCCAACATGGTGAAACCTCATCTCTACTAAAA
ATTAGCCAGGAGTGGTCCCACAGGCCTGTAATCCCAGCTACTTGGGGAGGCTAAGGCAGAAGAATCACTT
GAACCCGGGAGGCAGAGGTTGCAGTGAGCCGAGACGGCACCACTGCCACTCCAGCCTGGGCGACAGAGTG
AGACTCTGTCTCGACAAAACAAAAAAACCCTTGAAACAATATATTCAAGTCAACAGAACATGGCCATGTG
CCTCACACATCAGCATCATGGGAACACTGGCACTTCAGAGGCCAGTGGAAAAGTGGCAGTCCCGTTGGGG
CTGCATGGCTGGCAGCTGACCACAAGCCCACACAGCGCTTAGGAGTCAAGTGCAGCAGTGATGGAGCTCA
CACTACTGAGAGAGCAGGGAAACCACACTGAAAGGGCACGTTTCTTAACCTCAGAATGGGGCTACTAGCC
TCTAAAGCAGGAATTGCGTTTTGTTTAGTATTTCCATGGTCTGCTGCAAGGCGTGGCCTTTACCCAATGG
ATAAATGCGTACAAGGCTCTTGTGAGCAGTCAAGTTTCTCGAGGTTTACAGTTGAAGGGAAGTGGGATTG
TTTTCCTGCGCATTTAAATGAAGGTAGGTGGGTGATCACCTTTCCTTAAATGTGTGAAGGGATGAGATAA
AGAGATAGGCATCTTAATTGCCACTGATGGCCTTCAGGTGAGGACAGGCATGAGCCAACTGAAGCTTTGA
CAATTGTGCTGAACCCAAAACTTCAAAAACAAGAAAAAACATAGACTGGCTGAAATGATCTAAGTCAACA
GAGCATGGCCAGCGCTTCATACAAGGCAGGACCACAGGGGAACACTGACAGCCCAGGAGGCACTGAGACA
GAGGCAGTGGGAAGAAGTGACAGACCCCAGGGACTCCCCACCAACAGCAGCTGCTGTTGATTAGGAACCC
CCAGTAGACTGTCAGGCACCTGGTAGTGGAGAGGCTACCAAGGCCCGGACTGGAGAGGAGCCAAAGGAAG
AAACAGTGCAGTGCTTAGACCCCTCTGGGTCTGCCCGTGTCCATACCCACTAGGGAGATTCCATTCCAGA
AGTGGACATATTCACCAACAGAGTGCCTGGGGCTCACTCATCACAGCTGCCCCTCCATGAGGGCATCTCA
CTGCAGCCTTAGCAGGGAACAGGGTCATTTGCATTAGGCAGCTGCTGTCCTAGGAGGTCTCGGTGTCCAT
ACACTGCCATGTTCCCAAGGTGTTTCAAGCTCAAAAGTCTCATCTATGTCATACAGTCGTGCCTAGTTGG
CCCGTCTCCAAGACCTGACATCCATCCTTCTACTCTGGCCACACCTGCCCATCTCATGAAATGGGCCTAC
TTTAGTCAAATGCCCTCAGGGCCACAAAAGGGCACCACCTGAGTGGAGAGAGCACATTTGACCTCCCATC
AAAACCCTTCCCAGTTCCCAAAGACTAGTCACCATGACCTCATCCTGGCCCAAAGCCACAAGAACGGAAT
GCCTGCTACTAGTACAGGAGTAAGAAGCAAGGTCAGAGTTCCCATGCCTGCTGACTTGACCCTGTCAGAA
AAACTCAGCAGGGTGTGTGCCCCAAGAGACTTTAAAAACAAAGTAAAGGCACACGTGACACTCCAGGCCA
GATCATCTCTAGGGGTGCCATGCGACTGACAAAAGACTTATTAAGCATGCAGATTCCTGAGGCAATACCA
GACTTGCTATTGAATCAAATTGTCTGAGAGTAGGGTCCTCAAATCAGTTTAACAAGCATCGCAGGTAATT
CTGATGCCCACTAACATTTGAGACCAGTGTTCCAGATAGAATTATCCTTTTTTGGAAGACCTTCTTTAGG
CCTTGAATCAAACTCAAACTTGCAGCCCTTCTTCAGTCACTTTTTCTGGTGTGCTGCTTCCTCCAATAAA
GTAAGATATTAACCAGAAAAAAATAAAAGAGGCTGAGAGGGACACATGCCTCTAACAGAAGCATCAAGCC
ACTGACTTTTATTCCCATGAGCTCCTACTATGATGACATAACATGTCACATTTATTGCTCTTACCTACTT
CCTAAGTTACTGCTTGCCTAATAAGGAAAAGGGCAAGAAATAAGAGTCCAAAAGACACTCAGAACACCAG
CAGCAGCTTTCAAGTAAGGACTCAGGGTCAGGGTCTCACAACTCCTCCATTTCATGTTTCATTTACATTG
TCTTCGTGCTCCAGCAGCAAGGCTGACATGTGCTTTGGGGTAGGAGCCTGGAGTATTTCTGCCAGATGGT
CTGAGGTGACGTATACTCCTAGAGCCCAAGTGACCACCAGAAGGGACATGTGAGACAGCAGGCAGAGGCC
ATACATGGAGGATCACACTCCCTCAGACGTCATCAGCCCCATGCTTTATCCTTTGACTCCCCCTGATCCT
CAAGGCCAGGTGGGGTGCCCCTGCTGCATGCTCCAGAGAACTGTGGGCCTCTCCTACCATGGCTCTCAAT
GCACTGTTCCATAACTGGCTTCTCCCCTGGACAGAGCCACCCTGGTCTCCTTGTAAGCTTTAGTTGGGCT
TGTCCCTGAGTGTGTGGGGTAGGAATAGGAGTGTGTTCTTGTGTATGTTGCAATCTTTAGGCTCAGGAGA
CAAGGAGTTAGACCTGAGTCCTGACATGTGCACTCATTTGTACTCACCATTACATTCTATTTAATTGCTT
TGAGCCAGACAGGCTGTTTTCTTTACCATGAAATATACTAATCATTCAAAAAAGCCTTGTGTTTAAGAAG
ACAGCAAAAAACATCACTTCAAAAAGGCAACTTTTTTTTTTTCTTGGCATCAATGAATATGCACTTCAGA
AATCTACAGAGAACTTTTTTCACTTTGTATTTGTTCCACAGCATTCTAGCCAGGAGTACAAAATAGGAGC
TCTTCAAGCAGCACATGAAGATGGCCCAGGAGCCTTCAGCCATCCAGAGCAGCAGAGCAGCACCTAAATC
TCCACACACTTCCCAGTATAACCACTAAGAACACACTAGACCCTTGGCATTAGGGGATTTATCATTTCCA
GTTCTATTATCTGGGAATGACTCCAAGGGTTTGTGACAAGCTTTTGAAACGTAAGTGCTAAGACGAGTGT
GGACAAGTCACTGAGCTAATGAAGAATAGACCCACTGTCTGCTTGGCTCTGCTATTTTGTACCTGTCTTC
ACATGTGGGGAAACTTCATAACTGTGCCCACAAATCAGAGAAATTCGCAAAGGACTAGACAATGTATTGC
CTGTGAACTTCAGAGGCAACTGGAAATAGTGCAACTGGAAATCAACTAGCTTCATACATAGGCGGATTTG
TTTCCAGGGACCAAAAGCACAGAAGATAAGAAAACTTTTCAGGTGATGGCCATATTCTCATCATATCCTC
TCTGTGTGGAGTCTGCCTGTTGTCACAAAAACCTTGACCCTACATCAAGTTACACCTTAACAAAGGGAAG
ATACAGGCATCAGATAAAAGGTACTTGTTTGAAAGGCAGCCATAAGGGAGAACTGAACTTAAAAAAAAAA
AAAAAAAAAATTCCAAGCTGGTTTCAACAGTACTTTGTTTCCAGAACAAAGAAATGTTTCTAACCACATC
TTGTACCCCTTCCTCATCAACTCCAGACTACCACAGACCTTTTTCCAAAACTGTGTGTCACACATCCAGG
TCTTGTGCTTTAGAGCTGCCTCTCAGGCAATTTTAGCCAGCCATTTCTCCAAGTCCTGGATGTCAGCAGA
GCCCACGTCCCCTCTTCCACCCTTGGCACTGCACTCCAGGAACTCCACTTTGAGGGGCAACTGTGAGAAT
TCAAACTCTTTGCCTTTCTTCCCCAGCTGAGCAGGGGCAGTGCTGGAACTGTCCAGTGTGCTGGGGGCAG
CAGAACGGGTAACTCGTAAGGTGTTGCTGTGGGGCAAAAGGAAAGAAGTCAGGAGGCAAAATTTTTATAC
AGCAATTAAATCATATTAGTTTCTAAACATTTCAGCACTCATTTAAAACTTAACTATAGAATTGCCAATT
ATGCTATTAAGATTGTTGAATAATCCTTTGCATTTATAAGTCCCTCTCTTGGACAGAGAGGTGAGGTAGG
TGGGGCGGGCTGGGGGGGTCAGACCTCACAGGGCTAGAAGGCAGGTCAGCAGTAGACCAGGTCACACCAA
CTTTCTTTCAGCCCAAGGCCCCCTTCTAAGAGGATCTACTGAATGAAGCCGTCTAAAAATATATTTTTAA
AATACATGCTTTAAGTGTTATAAAAACATAAGTTTCTGAAAGTTAAGTGTGGGTAGATAGGGTATGTATT
TTTATCTTGAGTCTATTTGGATATGGAGTTTAGAAATCCACCAACTCCAGATTCTCATATCTAACTAGGG
CAGAATTACTATTATCCAACCTCTTTAACCACCAAAACCTTCCCAATGTAAGTCCCCTTACTGCCACCTG
CTGGCCCTAATAAAGGGTGCATAAGCAACACTCATTTGTCGGCCTCCCCTTCCCCTTTCTGCTATCCAGA
AGCAGAGGAATACATGCTGCCAATCTTCTTTCTTACAAAATAAAGCATTTCTTGTTCTGTAAAGTTGCTT
CAAAATTAGTGAAGAATCCACAGGCAAAACTAAGCAATACCTTGTTTAGGAAGGAACACACACACACACA
CACACACACACACACACACACACTGTAAAAGTATTTTTAAAAAACAATACAAAACTGGATATTGCTGACC
TCTTGAGGGAAAAGCTTTTGGGGAGAAACAGGGGCCTTCTAGGCATGGTGGTGAGTACATAGGTATTCAT
TTTAATATTTTTTAAGCTTAATGCATGTGACATATATACTATTATAAGTAAGATATTTTACAATAAAAAT
TTTTTAAGGAAAAAAGTAAAACATGTATTCAGCTTTTGCCACAAGGCCATGTAAATATAATATACCGTGT
TATTGAGAGACTATGAAATGCCTCTAAGCTAACAGGGCCTAATTCTCATAAATCTGAGATTAAAATTAGT
CAGATTCCGAATTTTAACTGATGTTAACCTTTACAATTTGTCAACATTTTGTTTCACTGCCTTTTAAGAA
AGCATTGCCTTTCAATGCTTTTAAAAAAGGATTGGCTTCTGTATTGTTTTTCTCTAAGTCTATAGAGTGA
AAAGCAAACAGCCTATGGAAAACATGTGTTTTAACCACGGGAGACTTGGACTGTACTGTTAAAAATAACC
CAAGACTGATTCACAAATAACATTATGACAGTTTTGGCTTCCCCAAAACAATTCCCAACAATTTCAAGAG
AATTTTTAGAAAAACCCAACCATCCAATTGTTCCAATGTCCTTATTAACCTAATGGCAGCAACAGATACA
ATCCAAGAGAGTGAGCATCAAGAGCACAGGAGGACAGGGTGATAAACTCGCAGCTCCAGACGGGAAAGGA
ACCACTGAAAATGGCATAGGCAGGGCTGGCAAAATTGTTCTGCAAAGAGCCAGAAATTAAATATTTTGTC
GCATATTTTTCTTCTGTTTTTTACCTGCCCTTTAAAAATGTCGATTTACTCTTGGCTTGCAGGCCCAGGC
AGGAGTTTGCCAATGCCCGAAGCTGACTAGATTCTACCAGACTCTAAGCTCCTGAGTACAGGAACACATT
TCCTCCTACAGCTGCTCCCAACTGCAGTCTTGGCATCCACTAGATGCACTACAGGTGCTTCTTAAATGAA
TAAGGGAGTCAGTAAGTCATAGTGGGGAGAAAAGTTTCTAAAAGTTGAATTTATACAGTTTTTCCTCATC
AGATCCTTTTCAAGGCCTTACACAAAAGCAATAAGAAAACATAACTTGCTTTACTATAAATGTTGATCAT
TTCTTCTACACCAGCTTTTGGCTAGCTCTTACAAATTGGCATAACGCAGAAGACATGCTAGATGCCTTAA
AATTCAAAAGCTTATAGCTCAATTTACAAACCTTGAAACACTCAAAGCAGGATATATAATATTGCCAAGT
ATTTTAACAATCTAAGAAGCTATAATGTAGTATCAAAGACTTGTAGTCTTCCTCAGTGTTCTGTTCTTAT
TCTGTCAACAATAATTTCCAAAGTGGCTTTTAAATGTTTTAATTTCTTTTTTCCTGATTCTCAGTTTTTA
TTTTTTATTATTTACATGCATCCTTAAATACCACTTCACAGGCATTATTGAGTGGTGAACAAACACTTCC
ACTCACTGAAATAACACCGTTCTCAAGTTGGCATGTAAACTTTTTTAGCTTCTAAAACCTTTCAACTATA
AATGATTTAGTAACAATGGTAAAATAAATTAAGTTTATGGTTTCAAAAAAATCTTCTTTCCATGAAATTT
TAAAATATCTATAAGCACCCTAGTAAGTTTTTGTGATTCCATTATTTTGTAAATTATTTAACAAATATTT
ATCACACTAATCTTCAGTCTATAATTGTGATAATCACATAAAACCAAACAAAAGAAACATGAATATGCTG
CTTACAGTCTAAGTGTTAAGATATATAATTAACCAACAGGCCTCTTTCACACTTACAGTTCTTTCTCCAG
CTGCTGTTGAATTAACTTTGCTGATTTTGCCATTGCAATATCTGTAAAGAAATAAAGACACTGTTGTAGT
TTAAACGAGAATGAGTTTTTTCCCCAAGACTTCTTACACAGAAGGAAAATTCCTATTTAAACCAGTAAGT
TTTTCTCTTTTTTTAAAAAAAGACCTTTTATCACTAGTTCCTTCTCTATCAAAGTAAGCTTTATACTTGC
ACTAGGGGCTTTCCCAAGATAATGCTTATAAACATATTATGCTGGTGACCCTAAAACTGACTTTAGCTTT
CCCTAAAGTTCTCCACACCTTATAGACCTTTCAATTCAAAACATATAAGTTTACTCAGTTTAACAGAAAG
AAAACCTACCAGTGCTGTGATTATGAGAAGTGCCTTCACATGCCAAAAGACAGTGCCCTATCTGTCATTT
TAAAGTAAACTTAGCATGTTTCCAATTGATCCTAAGACGGTATGTTTAAAGCATTAAGTGTACTAAAGGT
AAGAAACAAGCTCTGGTTTGGCACAAAGCCCATGGCAAATAAACAATAACTCCCTTCCTTATATTCAATC
AGGGAAATAAAGAGGTCAGGTAATGACGCTAATTGCATTCCCAATAAGGCATTATCAATATTATCTGGTC
ACTTCTGAGAAATGCCTTGAACTCTTCATAGAATCAGAATTTCAGATCTGGAAGAGACCTGAAGAGATCC
ACTAGTCTGTCTTATTGTACAGGTAACGGAACTGAAGCCCAGTTTTATTGGGGATTACTAGAAAAGTTTT
CAATGGTTATCAGGTTAAAAGAAAACAACTTCAGCAAACTTCCCTTGATAAAATGTGCTGTACAGTAAAC
TTGTTTGAAAGCAGAAACTCCACCCTGCAAAACTGTGCATCTGATATCACATATTGTCAATCTGATGAAC
TTAACTTGGTAAAGTAAAATAATTTATAATCAGAAAAAACACTGTATCAAAGAAAAATCAGTTAGCAGTC
ACTTGGTTATCATTCCTTGGTCTTCTGGTTAGAAGACTGGTGAAAAGGTAGGAGTAGAAATAAATTTTAA
GATTAAAAATCCTTCAATTTATAGTTCTTTACAGCAATTGTATCTCATCCTCAATAATAACAACTGTAAA
TAATGGAAATAATTCTTAGTAGCAGCAATCCCCAAAAAACTCAATTATTATTGCAAGAAAACATGATGGC
ACCTTGCTTATTGCAGGCTATTAAGAATGATGGTGTATTCTTCAGACCCATACTGTCAATGAGGACTTGA
TACAGAAACTCAGCCACATCTTTCACCTCTCGCTGGAATGCTGCACTATCCACAACAAACACAATAGCCC
TGGAGGGAGAAAACAAGAAAAACTGGCATGTGAACGTGTAACAGGACATCATTATCTTGAAAAACTATCC
TGAACTTCTTCAAAGTCAATCTTGGTGATTAACACTCAGCAGACACACATGCAGGTCTGTGCATATGTTA
TCATATAACATAAATCCTCATTTAACAAGGAACTATTGAGTGCTACTGTGTATCAATGACTGAAGCTGAA
AAAAGACAGTAAGAAATGATGCCTGTCCTCCAAGACAAATCATCATATAGAGAACCATATGATAAATGCC
AAGACAAAACTATGCACATGTGTAACAGGAATAAAGAGAAAGATCATTTAAATGAGACCAGGTCAAAGGC
AACTTTAAGGAGAATGTAACAGTTGAGTTGAATCTTAGCTAGACCAAAAATTGGGGCACAGGCTGTCTGG
AAAGTACAGGTTGAGTATCCCCAATCCAAAAATCCAAAGTCTAAAATCCAAAACACTTCTGGTTCCAAGC
ATTTTGGATAAGGGATACTCAGCCTACAGTATGTTCAAGCCACAAATACGTGAAACACATTCCCTGGGGA
AGTCAAGGTAAGCTGGTACACAGGGAGATGGGGAAATGAAGGAGAACCAAGTCAGACAGTGAGGCGTGAC
ACAATGACCTGTGGAGTTCATGCTTTTCTGTCTTATGAAGAATATTGGTGCCTAAATGGAAGACAAAGCA
ATCTTAGAGGGCGCTTGAACTAGAAGCACAGTGACCAGGTAGGGCCTGAACTAAGGCAGCCATAGTGGGA
CTCCTCATGTGACAGAAACAAAGAGATATCAAAGCTAAAGTGCAGCAGCCCAGGGAAACTGCAGACAACA
GGTCTTAGGGGGCCGGGTGCGGTGGCTCACGCCTGTAATCCCAGCACTTTGGGGGGCCGAGACAGGCAGA
TCACTTGAGGTCAGGAGTTCGAGACAAGTCTGGGCAACATGATGAAACCCAATCTCTACTAAAAATATAA
CAATTAGCCAGATATGGTGGCGGGAGCCTGTAATCCCAGCTACTTGAGAGTCTGAGGCAGAAGAGTTACT
TGAACCCAGAAGGCGAAGGTTGCAGTGAACTGAGATCATGCCACTGCACTCCAGCCTGGGTGACAAAGCG
AAACTCCATCTCAAAGAAAACAAAAAAACAAAAAAACCGGCCAGGCGCGGTGGCTCACGCCTGTAATCCC
AGCACTTTGGGAAGCCGAGGCGGGCGGATCACGAGGTCAGGAGATCGAGACCATCCTGGCTAACACGGCG
AAACCCTGTCTCTCCTAAAAATACAAAAAATTAGCTTGGTGTGATGGCGGGTGCCTGTTGTCCCAGCTAC
TCAGGGGACTGAGGCAGGAGAATGGCGTAAACCTGGGAGGCAGAGCTTGCAGCGAGCCAAGATCGCGCCA
CTGCACTCCAGCCTGGGCAACAGAGCGAGACTCCGTCTCAAAAAAAAAAAAAAAAATACAAGCCTTATGG
ACAAGGGTTTGAATGCCCAGGTGGAGATAAGAGACATGCAATGGCCAACATGCAGCTGGGGAGCTAGAAC
TGAATGCCCTGCACAAAGCCTACAACTTAAAATAGTCTCAGGGAGGTCGGGGCTTGCAAAAAAAGTTCCC
TTAGAAACAGCGGGCCTCCACTGCCTCACACAGATATAGGGTATGAATTTATACTCTCTCCAGGTGTAAG
ACCCCCAAGGGGAAAGGCTTACATAAAAATTGGGCCAGACTTATTGAAGCCCTATGGCCCCAACAGAAGG
AACTATGAACCGCTTATGGAGCAAGGCTTTCATATCCTACGGGATTAAAGAACCACCACAATATCAATGA
TGTGTCACTCTTGAAGAAAGGTTCTGGAGCAAAAATTACAAGGCACAGAATAAAATAATTATGAGGGAGA
ATAACTGGTACCCCAAAGAGAATAATCAGCACCCCAAAACTAAGATAATAAAGCTATCTATAAAGTAAAT
GTTTAAAATTATTTAAGAAAATACTACAAACTACAATAAAAAATAGAATAGTATAAAAAGAAACAGGCTG
GTTTGAAAAGGTAAGTATTTTAGACAAATATAGTAAATGATATTAATAAATCAATGGAAAAGTTAAG

### Example 3: Sequence Analysis

Methods for sequencing nucleic acids are well known in the art. Two methods of sequencing were used to identify the genomic DNA identified and described herein: Sanger sequencing and next-generation sequencing.

Sanger sequencing is a method of DNA sequencing based on the selective incorporation of chain-terminating dideoxynucleotides by DNA polymerase during in vitro DNA replication. Sanger sequencing requires single-stranded DNA template, DNA primer, DNA polymerase, normal dNTPs and modified nucleotides (i.e., dideoxy-NTPs, ddNTPs) that terminate DNA strand elongation. The ddNTPs are labeled with fluorescent dyes, such that each of the nucleotides (A, T, G, C) emit light at different wavelength, thereby differentiating between the different nucleotides. The DNA sample and reagents were mixed together in one reaction mixture, and elongation products were detected by emitted wavelength by automatic detection programs to resolve the nucleic acid sequence.

Next generation sequencing allows high-throughput sequencing to read thousands of sequences concurrently. Next-generation sequencing include Illumina and 454-pyrosequencing methods.

### Example 4: RNA Analysis from Ocular Samples

Retinas and whole-eye samples were isolated from the Nrv−/− mice using methods known in the art. RNA was then extracted using methods known in the art. Complementary DNA is produced from the extracted RNA, for example, by reverse transcribing mRNA (Superscript® cDNA synthesis kit, Invitrogen). Primers were designed to hybridize and detect the nucleic acid sequences described herein. A list of the primers and other useful oligonucleotides are included in Table 1.

Genetic mutations in the RNA sequences are detected by RT-PCR. The mutations are correlated to open reading frames (i.e., sequences encoding a polypeptide) or to a regulatory region (i.e., promoter regions, operator regions, suppressor regions, repressor regions) using methods known in the art, such as sequence alignment, and computer algorithms. Once the genetic mutation in the regulatory region or gene is identified, further validation assays are performed to elucidate the role of the regulatory region or gene in the progression of retinal vascular disease.

In summary, gene identification of Nrv1 mouse model of retinal neovascularization revealed: 1) linkage to mouse Chromosome 9; 2) genetic fine mapping to 1.0 Mb region of Chromosome 9; 3) sequence analysis of the candidate genes described above; and 4) correlation of genetic mutation to gene or regulatory sequence.

Sequence analysis, northern blot analysis, and functional assays are performed to identify specific gene regions that are responsible for the mutant phenotype described herein.

TABLE 1
Primers

| Name | Sequence | SEQ ID NO: |
|---|---|---|
| CVd3-NB-F | CATGAATCTGGCATGGTGTC | 26 |
| CVd3-NB-R | ATGCTCTCAAGCTGGGAAAA | 27 |
| CDV3 mRNAvr1 F | TTGACCCAGAGCTCGGAGC | 28 |
| CDV3 R5 | TCCGGACCTAGCACCAGAG | 29 |
| Cdv3 exon 3 F | TGAAAAGGAAGATGATGATAATG | 30 |
| Cdv3 intron 3 R | TCTGCTGAGAATGTAAGCTTC | 31 |
| Gap1 F | CATTTCCTGTTCCTCTGGCC | 32 |
| Gap1 R | TGAGGTTAGTAGCCAGGCAC | 33 |
| Gap2 F | CCCAACCTGACAGCACCAG | 34 |
| Gap2 R | GTACCTGCTCCTGACTGCC | 35 |
| Gap3 F | TTCAGTGGTAGGGGAAGTGG | 36 |
| Gap3 R | ATTTGCATGCTTTAGATTGGGG | 37 |
| Gap4 F | GGGATCCCATGCCCTCTTC | 38 |
| Gap4 R | GGAGCATGCTACTAACATGTG | 39 |
| Gap5 F | CATTGGTTGTGAGCCTAGCC | 40 |
| Gap5 R | TTCTGAAATTAGTACCACTGTAG | 41 |
| Gap6 F | CGGGAGCCATGCAGCCCAC | 42 |
| Gap6 R | AACGCTTGGCCACCCTCGG | 43 |
| Gap7 F | ACCACATGGTGGCTCAGAAC | 44 |
| Gap7 R | CTAATTGTCCTAGCTAGCTTAC | 45 |
| Gap8 F | GGGCATTTCTGAGTTCAAG | 46 |
| Gap8 R | CAGGGATATAATTGAAAGGTATG | 47 |
| Gap9 F | AAGACAGCTACAGTGTACTCAT | 48 |
| Gap9 R | TATCATGAATAGTGTCAAGCAGT | 49 |
| Gap10 F | TTCGCGGTGGAGAATTATCTC | 50 |
| Gap10 R | TGGAATAAACCCTCCTTGTTTG | 51 |
| Gap11 F | TTTTGAGACAGACTCCCTGGT | 52 |
| Gap11 R | CAGCGGAAGCTGGCCTACA | 53 |
| Gap12 F | GGTTGGTTCTCAGTGCCTAC | 54 |
| Gap12 R | ATCTGCCTGCCTCTCTCTATT | 55 |
| Gap13 F | TGTGGGAAGCCACAGAACTC | 56 |
| Gap13 R | CTAACCTCCCGCTGGAACC | 57 |
| Gap14 F | CTCCAACACATCTTTCTCAACT | 58 |
| Gap14 R | ACTTCACACTCATCAGCACAG | 59 |
| Gap15 F | CAGGGCTACACAGAGAAACC | 60 |
| Gap15 R | AGTGGCCTTCTGAGTGATTAG | 61 |
| Gap16 F | ATCCTAGCAAGTATTTATCTCTC | 62 |
| Gap16 R | CCAGCACTCGGGAGGCAG | 63 |
| Gap17 F | CATGCAGGGGATTTGTCAATG | 64 |
| Gap17 R | CTGGTGTGTCTGAAAAGAGTG | 65 |
| Gap18 F | AATGCTGTAAATGCAAGCCTTC | 66 |
| Gap18 R | TACACAGAGAAACCCTGTCTC | 67 |
| Gap19 F | ACATGCGTGCGCGCCCTC | 68 |
| Gap19 R | CTAAGGAGCGGCCACCCG | 69 |
| Gap20 F | AGCTCTTGTCTCTAGCTGCGT | 70 |
| Gap20 R | GATGCTATACCCTCATACAGG | 71 |
| Gap21 F | TTTGTAGAAGCCCCGCTCAC | 72 |
| Gap21 R | TCCTCGGCTTGCTTCCATCA | 73 |
| Gap22 F | GCCCTGGATGTCCTGGAAC | 74 |
| Gap22 R | ACCACCTAGTATACTGCAGAC | 75 |
| Gap23 F | TAGAAGTGTGTCACACCACAG | 76 |
| Gap23 R | GCTAGGGATGTCACTCAGTG | 77 |
| Gap24 F | ACCAAGAGAATGTCAGTGTAAG | 78 |
| Gap24 R | CTTGTTAGAGGAGTGTGTCAC | 79 |
| Gap25 F | CTGTGATACCCAGATGTTTTG | 80 |
| Gap25 R | CCCACAATACTTATAGATTATAG | 81 |
| Gap26 F | CACTGCTCATCTACACAAAGC | 82 |
| Gap26 R | CCAACAGCTATCTGTACAGC | 83 |
| Gap27 F | ATCTGTAAGAGTCCAGAGCAG | 84 |
| Gap27 R | GTTGCTAGTATCTGTCACCC | 85 |
| Gap28 F | TGTGAATGGAAGTCCAAGGATC | 86 |
| Gap28 R | TGGGAGTGCTGTCCTCAGTT | 87 |
| Gap29 F | CAGAAGGTAGCGCCTTCTTG | 88 |
| Gap29 R | CTCCTGGGCTTCTTACAGTTC | 89 |
| Gap30 F | GCCTCACCACTATCGAGAAG | 90 |
| Gap30 R | ATCTGTAACTCCAGTTCCAGG | 91 |
| Gap31 F | AGCCTTGGGTGAGCAAATGG | 92 |
| Gap31 R | GCTGTCCTAGAACTCACTCT | 93 |
| Gap32 F | AGAGTTCGAGACCAGCCTG | 94 |
| Gap32 R | TCTGCTGGCTGCTTCCTTG | 95 |
| Gap33 F | AGGGCAGAGCAAGACGCTC | 96 |
| Gap33 R | TGCGGACATGGTGCTGGCA | 97 |
| Gap34 F | GGGAGGACCAAGGGGGAAA | 98 |
| Gap34 R | TTGGGGTGGGGGCAACCAG | 99 |
| Gap35 F | TCTGATACCTGTGGGAAGGAG | 100 |
| Gap35 R | CATAGCTGCACGGTGCCTG | 101 |
| Gap36 F | GAGCACATACAAACTATCACAAG | 102 |
| Gap36 R | TACCAGGGAGCTAGCTGTAG | 102 |
| Gap37 F | TGTCCCTCATGGGAACAGTG | 103 |
| Gap37 R | GACTGTCAGCAAAGCTGCTGGTG | 104 |
| Gap38 F | GCTCCAGCAGATCCAAAGC | 105 |
| Gap38 R | AACTCCTCCACGGCTACATTT | 106 |
| Gap39 F | ACCAGGATGACACTTGGGTG | 107 |
| Gap39 R | CTGCACTCAGAAGACACTAAG | 108 |
| Gap40 F | GGCAGGAACGGAAGCAGAGG | 109 |
| Gap40 R | GTGGCTCATGCCTTTAATCC | 110 |
| Gap41 F | CACAACTGTCTGTAACTCCAG | 111 |
| Gap41 R | TGTGTGCCCTGTGCGGATG | 112 |
| Gap5Fbis | TTTTGTGAGACTAGGCCGGG | 113 |
| Gap5Rbis | AAGTGCTTCTAAAGCTGGCCA | 114 |
| Gap7Fbis | AGATGGCTCAGCGGTTAAGA | 115 |
| Gap7Rbis | CTAATTGTCCTAGCTAGCTTAG | 116 |
| Gap8Fbis | GGGCAGATTTCTGAGTTCAAG | 117 |
| Gap8Ftris | AAGCCAGCCTTGAGGAAGAC | 118 |
| Gap8Rtris | TGTGTGCATTTTTACAGCCACT | 119 |
| Gap9Fbis | AAGCCAGCCTTGAGGAAGAC | 120 |
| Gap9Rbis | TGTGTGCATTTTTACAGCCACT | 121 |
| Gap13 Rbis | CTAACCTCCCGCTGGAAGG | 122 |
| Gap15 Rbis | TGACTGTGCTGCACTTGGAT | 123 |
| GAP19Fbis | TTTCAGCCTGGCAAACAACG | 124 |
| GAP19Rbis | CGCTAACCAGAAAGCAAGCG | 125 |
| GAP21Fbis | CCCCGCTCACAGTGTCATAA | 126 |
| GAP21Rbis | ATCCTTCCTCGGCTTGCTTC | 127 |
| GAP22Fbis | CTCAGAGCACCTCAGAGCAC | 128 |
| GAP22Rbis | GCAGTGACAAGTCCCTCCTC | 129 |
| GAP30Fbis | CTTCAAGAGCAGCCTCACCA | 130 |
| GAP30Rbis | GGTTTGTTTCCTTGCCGTCC | 131 |
| GAP32Fbis | CAGCCTATGATGGGAGCCAC | 132 |
| GAP32Rbis | CCCAAGGCTTGCCCTAACTT | 133 |
| GAP33Fbis | GTGGGTGGGTGAGACAGAAG | 134 |
| GAP33Rbis | TTCCCAAAATCTCCCCCTGC | 135 |
| GAP36Fbis | CTGCTAACCACTGGCTCTGT | 136 |
| GAP36Rbis | GAGCTAGCTGTAGGGCAGTG | 137 |
| GAP37Fbis | TGTTGAACCACCATCCCTGG | 138 |
| GAP37Rbis | ATGCCCAAGGTCTCCAAGTG | 139 |
| GAP41Fbis | CTCCCAGTGAACACCAGCAT | 140 |
| GAP41Rbis | TGGAAAGATGTTCCCCAGCC | 141 |
| GapC1F | TGCCTTGAACAGTGGAACATG | 142 |
| GapC2F | CCATCCCTCTCTCACTTGAG | 143 |
| GapC3F | TAGAGTGTAGGTTAGTACTCTTA | 144 |
| GapC4F | AGCACTTGCCATAAGCATTAGA | 145 |
| GapC4R | TCTAATGCTTATGGCAAGTGCT | 146 |
| GapC5F | ACAGAGGACTCAAGGTCAGC | 147 |
| GapC6F | ATCCACAAGCACTGTACAAACA | 148 |
| GapC7F | ATACGTAGGCAAAGCTGATCG | 149 |
| GapC7R | CGATCAGCTTTGCCTACGTAT | 150 |
| GapC8F | CTGCCCACCAGCTGGAAGT | 151 |
| GapC9F | CAATGTTAGGAACTGATTGCTC | 152 |
| GapC10F | ACACCTGCATACACTGAAGCA | 153 |
| GapC10R | TGCTTCAGTGTATGCAGGTGT | 154 |
| GapC11F | TTATAGATCAGACTTGGAGATAC | 155 |
| GapC12F | GTGAGAACACCTAGGGATATG | 156 |
| GapC13F | ATTCCAGCAGCTCAAGTTCAG | 157 |
| GapC13R | CTGAACTTGAGCTGCTGGAAT | 158 |
| GapC14F | AGCGAGTGAACTACAGGAGC | 159 |
| GapC15F | TCGCCAGGATTCTTGAGTCG | 160 |
| GapC16F | TCCGGAGACGTGCCTAATTC | 161 |
| GapC16R | GAATTAGGCACGTCTCCGGA | 162 |
| GapC17F | CACCTTCGTTATGGCCTTGC | 163 |
| GapC18F | TGCTCTGGTGGTACGTTCCT | 164 |
| GapC19R | TCACTAGGGACGTCACTCTC | 165 |
| Ky Ex1 F | GCCAGTGACTTCTCTTAGGGG | 166 |
| Ky Ex1 R | GTGATCAGCCTAAGGCCCTG | 167 |
| CDV3 F3 | CGCGCCTTCCTTGTCCCTCAG | 168 |
| CDV3 R3 | GGCTCAAGCGGGACCACGAG | 169 |
| CDV3 F4 | TGCTCCACGCTCGCGGTTC | 170 |
| CDV3 R4 | CGTGGCCGTGGTTGGGAGAG | 171 |
| CDV3 F5 | CTTCAGCCAAGTCACCAACC | 172 |
| CDV3 R5 | TCCGGACCTAGCACCAGAG | 173 |
| CDV3 F6 | CGTAGAAGGAACGGATTGTC | 174 |
| CDV3 R6 | GGACCCTCACCTTCGTTATG | 175 |
| CDV3 F5 | CTTCAGCCAAGTCACCAACC | 176 |
| CDV3 R6 | GGACCCTCACCTTCGTTATG | 177 |
| CDV3 F6 | CGTAGAAGGAACGGATTGTC | 178 |
| CDV3 R5 | TCCGGACCTAGCACCAGAG | 179 |
| CDV3 F7 | ACGTACCACCAGAGCACGG | 180 |
| EphRb1 var 2 QP F | ACAGGCCGAGGGGAGTT | 181 |
| EphRb1 var 2 QP R | CTCTTGGTGACGACACCCTC | 182 |
| Slco2a1 QP F | GGTGCCCATTCAGCCATTTG | 183 |
| Slco2a1 QP R | CACTGTAGCCGTGTCCACTC | 184 |
| 1300 RIK QP F | CGGGACATCTCGGCCAATAA | 185 |
| 1300 RIK QP R | TCAGTTGGAAGCCTGTTCCC | 186 |
| 7SK QP F | AGGTCAACCAAGCATGAAAAGAG | 187 |
| 7SK QP R | CTCCTGGCCTAGTGGCAATG | 188 |
| GM20425 QP | FCAACCTCACCCGAGATACCC | 189 |
| GM20425 QP | RTTACGTGGCTCTAGGCTCCA | 190 |
| GM5627 QP | FCCCATTATCTCCACCGTCCG | 191 |
| GM5627 QP | FCCACCCCTTCCGTGTATGAC | 192 |
| EphRb1 var 1 QP | FTGCAGCAGGAAACGAGCTTA | 193 |
| EphRb1 var 1 QP R | AATTCCCGGACGGCTTCATT | 194 |
| Ky RTPCR F | ATGGAGCTGAAGAAGGACAG | 195 |
| Cep63 RTPCR | FGCTGCGGCTAACTGTGGTG | 196 |
| Anapc13 RTPR | FGCCTGACAAGATCAAAGCTG | 197 |
| Amotl2 RTPCR F | ATTGTCTAGGCAGGTGTGTG | 198 |
| Gm5627RTPCR F | GCAATGTGAAGTCTCTGGGC | 199 |
| Gm5627RTPCR R | TACAGGTATCCTGTGCGTGGA | 200 |
| RyK RTPCR F | TGCTTGGTCTTGATGCAGAG | 201 |
| Rab6b RTPCR F | CCAGCTGCCAGCACCATGT | 202 |
| Srprb RTPCR F | CTTTCATGGCTTCGGCGAAC | 203 |
| Tfr RTPCR F | GAAGCGGGTCGGTCTGTAC | 204 |
| 1300RkenRTPCRF | GCAGAACGCAGTTCCACAG | 205 |
| 1300RkenRTPCRR | TCCACATGGAGGCAAGCAG | 206 |
| Topbp1 RTPRF | CTCGGGCTCCACCTAGTTC | 207 |
| 7SK RT F | TGCCATATCTGTCACCCCATTG | 208 |
| 7SK RT R | TCAAGCTCCTGGCCTAGTGG | 209 |
| SNORA RT F | TCCCTAGAGGTGTGACAGGC | 210 |
| SNORA RT R | GTTCATAGGTCAGAACACCAAGC | 211 |
| Gm20425 RT F | CAGAAGCGGGTCGGTCTGTA | 212 |
| Gm20425 RT R | GTCCACCCTTGGCACTACAC | 213 |
| Ephb1 RT F | CTGGATTGCTTGCTGCTCTTC | 214 |
| Ephb1 RT F | AAACCCAGACATGCTTCTGCT | 215 |
| Slco2a1 F | AAGCCTGAAGCACCCAAGTC | 216 |
| Srpbp F1 | CAATAACAGGGGTAACAGCC | 217 |
| Srpbp F2 | GCACACTGGACAGTTCCAG | 218 |
| Srpbp F3 | ACCCTGCAGTTTGAGAAAGG | 219 |
| Srpbp F4 | CTCGGTGACCATCCCACAC | 220 |
| Srpbp R 1 | GGCTGTTACCCCTGTTATGG | 221 |
| Srpbp R 2 | CTGTAGACCTTCACACCCC | 222 |
| Srpbp R 3 | CACTACACTCCAGGAACTCC | 223 |
| CDV3mRNAvr1 F | TTGACCCAGAGCTCGGAGC | 224 |
| CDV3mRNAvr1 Rbis | TCCATTGGTTTACTGTTTATTCAA | 225 |
| CDV3mRNAvr1 F | TTGACCCAGAGCTCGGAGC | 226 |
| CDV3mRNAvar3 R | GGTAGTATTCATTTTAAGTACTTC | 227 |
| CDV3mRNAvr4F | TTGAGTTGTGGAATTTCTGTCGC | 228 |
| CDV3mRNAvr1 Rbis | TCCATTGGTTTACTGTTTATTCAA | 229 |
| Cep63-NB-F | GACGCCCACATTGAAGAAC | 230 |
| Cep63-NB-R | AGCTATTCACATGTAAGTCGGAAA | 231 |
| Anapc13-NB-F | GGACAGACCTGGCCTTACAG | 232 |
| Anapc13-NB-R | ATTTGGCGACAGGAAACATT | 233 |
| Amotl2-NB-F | CTGCAGCCGGAGGCCTGTTG | 234 |
| Amotl2-NB-R | AACTGTGGCAGCAGGGGCAG | 235 |
| Ry-NB-F | GTGAATGACCAGTGAGGGGCTGC | 236 |
| Ry-NB-R | CACCAGGCAGGCATCGTGGG | 237 |
| Rab6b-NB-F | AGCAGTGCAGAGATTGTGAAAGGGC | 238 |
| Rab6b-NB-R | GGCTCCCAGTGCTGTGGCAA | 239 |
| RyK-NB-F | ATAGCCCAGCCAATCAACTG | 240 |
| RyK-NB-R | CACCCCCTAGTCCAAAGTCA | 241 |
| Slco1a1-NB-F | CCTTCAGCTGGGACTACTGC | 242 |
| Slco1a1-NB-R | CCCACCACTGAAGTGTTCCT | 243 |
| Trf-NB-F | AAAGAACCCTGCCGAATGGG | 244 |
| Trf-NB-R | AGCAGCGAAGACTACACCAG | 245 |
| Srprb-NB-F | GCATCGCTTTGAGAGTAGGG | 246 |
| Srprb-NB-R | GCTCCTAGAGCCACCTTGTG | 247 |
| BActin-NB-F | TGTTACCAACTGGGACGACA | 248 |
| BActin-NB-R | AAGGAAGGCTGGAAAAGAGC | 249 |
| Topbp1-NB-F | ACAACCACCCAACGAATGTT | 250 |
| Topbp1-NB-R | CAAAGTTTTGGTGCCCAAGT | 251 |
| CVd3-NB-F | CATGAATCTGGCATGGTGTC | 252 |
| CVd3-NB-R | ATGCTCTCAAGCTGGGAAAA | 253 |
| EphB1-NB-F | TGGCAGGCCATCAGAAGAAG | 254 |
| EphB1-NB-R | CCATGCAGTAGTGGTCAGCA | 255 |
| Rik 1300 NB-F | ATGTGACCAGTGGAGTGCTG | 256 |
| Rik 1300 NBR | CAGCTCCGACTGAAGAATGC | 257 |
| GM 20525 NB-F | TGGAGCCTAGAGCCACGTAA | 258 |
| GM 20525 NB-R | ACATCTTTCACCTCCCGCTG | 259 |

NMF128 Finemapping I

| | | |
|---|---|---|
| D9Mit182 For | GTGAAATTGGTTATGTAAATGTCTGA | 260 |
| D9Mit182 Rev | GAGATGACTAGGGTGAACTGGG | 261 |
| D9Mit115 For | TCCAGACTCCTGGGAACTACA | 262 |
| D9Mit115 Rev | TTTCCCAGCCAGTAAAGGC | 263 |
| D9Mit358 For | CCTAAGGTAATTAAATGTGTAAGACCA | 264 |
| D9Mit358 Rev | TTCTGTTTCACTATACATGTGCACA | 265 |
| D9Mit276 Fo | TTGGGACTGTTTGGAATGTG | 266 |
| D9Mit276 Rev | AATTCATAAGTGTCCCCTGGG | 267 |
| D9Mit347 For | CCTCCACATGTGCACTGCT | 268 |
| D9Mit347 Rev | CTGTCCATCTATCATCTATCTGTCTG | 269 |
| D9Mit242 For | AGAATTGCCTTCTGGTCTATACA | 270 |
| D9Mit242 Rev | CGTGTTAAACGTGAATATGTTTG | 271 |
| D9Mit183 For | TAAATACTGAGGCAGAGTGACAGC | 272 |
| D9Mit183 Rev | GGGAGCATTATCATTCCCAA | 273 |
| D9Mit355 For | CTCATTCACTTCCTGGTCCTG | 274 |
| D9Mit355 Rev | GAAGGAAAGCCCACACTTTG | 275 |
| D9Mit35 For | CCAGCGCACTGTTCTGATAA | 276 |
| D9Mit35 Rev | AGGTGCCTTCTGCTTTGAAA | 277 |
| D9Mit34 For | ATGTTAAAACATGGGCTGGTG | 278 |
| D9Mit34 Rev | TGCTTTCTTGTTATTTCATCTACG | 279 |
| D9Mit274 For | AATTTTTAAAGTCTCTCTCCTCTTTCG | 280 |
| D9Mi274 Rev | CCTCTGTTTCATCCTTGTACTGC | 281 |
| D9Mit344 For | GACCTATGACAGCAAGGGGA | 282 |
| D9Mit344 Rev | GGTCCATTTCTCCTCTTTTTTT | 283 |
| D9Mit273 For | CCACTGTGCTTTGGTGACTG | 284 |
| D9Mit273 Rev | GGATTCTGTGGAGGGCTGTA | 285 |
| D9Mit308 For | AACCATGTTCCAGTGGTAGACA | 286 |
| D9Mit308 Rev | TAACAATGAAAGTTTGTGTCCAGG | 287 |
| D9Mit291 For | CATCCCCTTTTACTCTGAGTGG | 288 |
| D9Mit291 Rev | CTAGCCTAGCAGACTTGATGAGC | 289 |
| D9Mit135 For | AGGAAAGAGCCAACTCCCAT | 290 |
| D9Mit135 Rev | TTTCCTCTCCTTTTTGCGTA | 291 |
| D9Mit77 F | AAGAAGGAGGTAGAGCATCGC | 292 |
| D9Mit77 R | CTTTCGGTAATTGCTTGCTACC | 293 |
| D9Mit359 F | ATCACAGAAATGAAGCAAAGACC | 294 |
| D9Mit359 R | TGTATTCTTCCCCCTTCTCTAGC | 295 |
| D9Mit310 F | AAATCTTTATCATGAAATAGGGATGC | 296 |
| D9Mit310 R | CCCACCCCATGTGTATCAC | 297 |
| D9NH8 F | GGTTCTAGAGATGGAACCCA | 298 |
| D9NH8 R | CGAAGCTGTAGAGTATCCCTG | 299 |
| D9 DBL1 F | GACGAGGAAGACACAGCCAA | 300 |
| D9 DBL1 R | ACCCCACTTCCCAAAACCTG | 301 |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1.-14. (canceled)

15. A method of detecting a genetic mutation in a subject comprising:
providing a test sample from a subject;
detecting a genetic mutation associated with centrosomal protein 63 (Cep63) gene or a regulatory region thereof and/or topoisomerase (DNA) II binding protein 1 (Topbp1) gene or a regulatory region thereof,
wherein said detecting comprises utilizing at least one primer selected from the group consisting of SEQ ID NOs: 196, 231, 207, and 251.

16. The method of claim 15, wherein said subject suffers from or is prone to suffer from retinal vascular disease.

17. The method of claim 15, wherein said detecting comprises utilizing primers as set forth in SEQ ID Nos: 196 and 231.

18. The method of claim 15, wherein said detecting comprises utilizing primers as set forth in SEQ ID Nos: 207 and 251.

19. The method of claim 16, wherein said retinal vascular disease is selected from the group consisting of diabetic retinopathy, age related macular degeneration, and retinopathy of prematurity.

20. The method of claim 15, wherein said test sample is obtained from a biological fluid selected from the group consisting of blood, vitreous humor, and aqueous humor.

21. The method of claim 15, wherein said Cep63 gene comprises a nucleic acid sequence of SEQ ID NO: 303.

22. The method of claim 15, wherein said Topbp1 gene comprises a nucleic acid sequence of SEQ ID NO: 310.

23. The method of claim 15, further comprising treating said subject using an anti-vascularization agent, an anti-angiogenesis agent, anti-inflammatory agent, or surgical procedures, when said genetic mutation is detected.
